(19) United States
(12) Patent Application Publication
(10) Pub. No.: US 2008/0167385 A1
(43) Pub. Date: Jul. 10, 2008

(54) Crystal Structure of Human Proliferating Cell Nuclear Antigen (Pcna) and Uses Thereof

(76) Inventors: George Kontopidis, Dundee (GB); Daniella I. Zheleva, Fife (GB); Campbell McInnes, Dundee (GB); Peter Martin Fischer, Arbroath (GB); Malcolm Douglas Walkinshaw, Edinburgh (GB)

Correspondence Address: LAHIVE & COCKFIELD, LLP, ONE POST OFFICE SQUARE, BOSTON, MA, 02109-2127, US

(21) Appl. No.: 11/596,149

(22) PCT Filed: May 10, 2005
(86) PCT No.: PCT/GB05/01764

§ 371 Date: Aug. 21, 2007

(30) Foreign Application Priority Data
May 11, 2004 (GB) 0410498.0

(51) Int. Cl. A61K 47/00; C07K 14/00; G01N 33/566; C40B 30/04; G06G 7/00; C12N 5/06; A61P 43/00

(52) U.S. Cl. 514/789; 530/350; 436/501; 506/9; 703/11; 435/375

(57) ABSTRACT

The present invention relates to crystals comprising human PCNA, and methods and assays for designing and identifying small molecule PCNA inhibitors using said crystals.

The present invention relates to proliferating cell nuclear antigen (PCNA) and small molecule inhibitors thereof. More specifically, the invention relates to crystals comprising human PCNA, and methods and assays for designing and identifying small molecule PCNA inhibitors using said crystals.

### BACKGROUND TO THE INVENTION

PCNA is an essential auxiliary protein for the processes of both DNA replication and repair. It stimulates the activity of DNA polymerase δ (pol δ) and increases its processivity¹ by acting as a clamp platform that slides along the DNA template². Apart from pol δ, PCNA associates with a host of other proteins, either involved directly in DNA replication and repair, or in the regulation of these processes³. The presence of a common PCNA-binding motif in such proteins suggests that regulation may depend largely on PCNA partner proteins competing with one another for access to PCNA.

Proteins involved in cell-cycle control, particularly the tumour suppressor protein p21 (also known as WAF1, CAP20, Cip1, and Sdi1), also participate in these interactions⁴. In particular, the induction of p21 after DNA damage leads to inhibition of cell-cycle progression and DNA replication. This effect is not only due to CDK inhibition, but also results from direct binding to PCNA, thereby interfering with PCNA-dependent DNA synthesis^{5,6}, while permitting DNA repair^{7,8}. p21 contains a CDK-binding site in its N-terminal region (residues 53-58), which is distinct from two cyclin-binding sites located in the N— and C-terminal regions respectively^{9,10}. The PCNA-binding motif present in the C-terminus of p21 has been characterized extensively^{11,12} and is conserved in many other PCNA protein partners³, thus supporting the notion that PCNA plays multiple roles in DNA replication and repair, as well as in cell-cycle regulation¹³. At the cellular level the competition between p21 and DNA replication factors for binding to PCNA is believed to be the mechanism through which DNA synthesis is inhibited. It is known that in cells PCNA and p21 can participate in quaternary complexes with CDK/cyclin pairs, particularly CDK4/cyclin D1^{14,15} probably contributing to the coordination of cell cycle progression and DNA replication¹⁶.

Deregulation of PCNA expression is a hallmark of many proliferative diseases and in the clinic PCNA serves as a general proliferative marker, especially in the prognosis of tumour development¹⁷. In fact PCNA expression levels are directly related to the malignancy of various tumours and antisense oligonucleotide-mediated suppression of PCNA expression was demonstrated to selectively inhibit gastric cancer cell proliferation in vitro and in vivo¹⁸. Antisense strategies targeting PCNA mRNA have also shown promise in models of other proliferative diseases, including glomerular nephritis¹⁹, restenosis²⁰ and rheumatoid arthritis²¹. The fact that PCNA is required absolutely for cell proliferation indicates that pharmacological modulation of PCNA function should not be able to be circumvented by compensatory pathways. Furthermore, the ablation of PCNA expression or function in cells under proliferative stimuli appears to constitute an apoptotic trigger¹⁷, suggesting that effective elimination of hyper-proliferative cells should be possible in a therapeutic setting. Collectively these facts indicate that PCNA may represent an attractive target for intervention in proliferative disease.

Interest in p21 as a starting point for the peptidomimetic design of anticancer agents has stemmed from observations that synthetic peptides derived from the C-terminus of this protein, when rendered cell-permeable²², are capable of arresting and killing cancer cells²³⁻²⁵. Interestingly, the C-terminus of p21 harbours overlapping recognition sites for both cyclins and PCNA. For the purposes of structure-based drug design there is therefore an interest in defining and delineating the relevant recognition sites in PCNA and cyclins responsible for the antiproliferative effects of the p21-derived peptides.

Previous studies have determined the structure-activity relationships of peptides related to p21(141-160) with respect to both PCNA- and cyclin binding^{26,27}. The p21 sequence in question is ¹⁴¹KRRQTSMTDFYHSKRRLIFS¹⁶⁰ (determinant residues for PCNA and cyclin interactions are shown in italics and bold face, respectively). Analysis of the motif conserved in many PCNA-binding proteins³ demonstrated that it takes the form QXXhXXaa, where X represents any amino acid, h indicates moderately hydrophobic residues, and a corresponds to aromatic hydrophobic residues; i.e. in the case of p21 ¹⁴⁴QTSMTDFY¹⁵¹ Ref.^{11,25}. As far as the interaction with G1- and S-phase cyclins (D, E, and A) is concerned, p21(141-160) peptide truncation and substitution studies showed that the sequence ¹⁵⁵RRLIF¹⁵⁹ was necessary and sufficient for effective inhibition of CDK4- and CDK2-associated kinase activity^{26,27}. Pentapeptides based on this minimal sequence represent a starting point for the development of a new class of selective CDK inhibitor drugs.

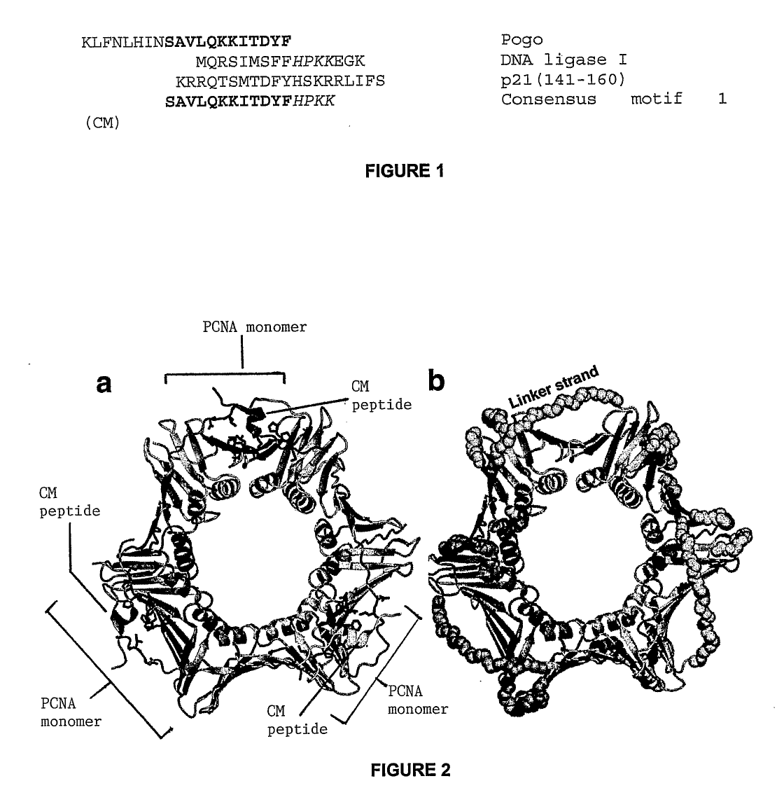

Truncation of p21(141-160) peptides from either terminus was not tolerated without a serious diminution of the binding affinity for PCNA. This suggested that it would not be possible to design small tight-binding peptides or peptide analogues that would inhibit PCNA. Furthermore, separation of the cyclin-binding properties from PCNA affinity appeared difficult. On the basis of the sequences of two particular PCNA binding proteins, the Pogo DNA transposase and DNA ligase I (FIG. 1) a number of consensus motif peptides were designed²⁸. One of these, a hybrid containing 12 residues from the C-terminus of Pogo, flanked by 4 residues from DNA ligase I, was found to bind to PCNA with an affinity very similar to that of the p21(141-160) peptide, but was unable to inhibit CDK function. This peptide, designated consensus motif (henceforth CM) peptide, was also an efficient inhibitor of PCNA-dependent DNA replication in vitro. Peptides derived from the consensus PCNA-binding motif of PCNA-binding proteins other than p21 have also been shown to result in antireplicative, antiproliferative and proapoptotic effects when transfected into cells²⁹⁻³¹.

The present invention seeks to elucidate structural information on the binding interactions between PCNA, p21, and CDK/cyclin. Specifically, the invention seeks to elucidate information on the 3-dimensional structure of the PCNA binding domain and the nature of the binding interactions between PCNA and compounds capable of modulating PCNA. The invention further seeks to provide assays and methods for identifying candidate compounds capable of modulating PCNA.

### STATEMENT OF INVENTION

The present invention relates to various crystal structures comprising human PCNA, and their use in the identification of compounds capable of binding to and/or modulating PCNA.

Specifically, binding studies carried out in the context of the present invention suggest the formation of a quaternary complex between PCNA, p21, and CDK/cyclin, in which a 20mer peptide is sufficient to mimic the assembly role of full-length p21. A structural model of the complex shows how p21 can act like double-sided tape to bind to both PCNA and cyclin/CDK. The invention also provides a complex structure of PCNA and the CM peptide, as well as the first X-ray structures of free human PCNA. These X-ray and model structures delineate a well-defined surface binding-pocket in PCNA that can be used for the design of inhibitors of PCNA-dependent DNA replication.

Aspects of the invention are presented in the accompanying claims and are further described in the following paragraphs.

### DETAILED DESCRIPTION

### Crystal

A first aspect of the invention relates to a crystal comprising human proliferating cell nuclear antigen (PCNA).

In one preferred embodiment, the crystal of the invention is human PCNA.

In one particularly preferred embodiment, the crystal is monoclinic. More preferably, the crystal is of space group C121.

In one preferred embodiment, the crystal comprises a unit cell having the following unit dimensions: a=136.6 Å, b=83.26 Å, c=71.63 Å.

In a highly preferred embodiment, the crystal comprises the atomic coordinates set forth in Table 3.

In another preferred embodiment of the invention, the crystal is trigonal. More preferably, the crystal is of space group P3.

In one preferred embodiment, the crystal comprises a unit cell having the following unit dimensions: a=82.89 Å, b=82.89 Å, c=70.86 Å.

In a highly preferred embodiment, the crystal comprises the atomic coordinates set forth in Table 4.

In another preferred embodiment, the crystal of the invention comprises human PCNA and a ligand.

Preferably, the ligand is a peptide structurally related to p21.

In one particularly preferred embodiment, the ligand is a peptide of formula I

1 16
SAVLQKKITDYFHPKK (I)

In one especially preferred embodiment, the crystal comprising human PCNA and a peptide of formula I comprises a unit cell having the following unit dimensions: a=119.1 Å, b=119.1 Å, c=305.82 Å.

Even more preferably, the crystal comprising human PCNA and a peptide of formula I comprises the structural coordinates set forth in Table 5.

More preferably still, the crystal comprises one or more of the following interactions between human PCNA and residues 3 to 15 of said peptide of formula I: V3(N)-I255(O), L4(N)-I255(O), L4(O)-I255(N), Q5(OE1)-W227(O), Q5(NE2)-A252(O), Q5(NE2)-P253(O), K6(N)-P253(O), K6(CG)-I255(CG1), I8(N)-H44(O), I8(CD1)-P234(CB), I8(CD1)-Y250(CB), I8(CD1)-Y250(C), I8(CG2)-L47(CD1), I8(CG1)-V45(C), Y11(CG)-P234(CD), Y11(CD1)-P234(CD), Y11(CZ)-P234(CD), Y11(CZ)-P234(CG), P12(CD2)-P129(CD), P12(CE2)-P129(CD), P12(CZ)-P234(CG), P12(CZ)-Y250(CD2), P12(CE1)-Y250(CD2), P12(CE1)-W364(O), H13(O)-G127(N), P14(CA)-L126(CD2) and K15(N)-Q125(O).

In one preferred embodiment, the peptide of formula I comprises one or more of the following intramolecular H-bonds: Q5(NE2)-K6(O), K7(O)-D10(N), I8(O)-Y11(N), I8(O)-F12(N) and D10(N)-D10(OD1).

In another preferred embodiment, the crystal of the invention as described above comprises a ligand binding domain.

Another aspect of the invention relates to a crystal comprising a human PCNA ligand binding domain. Preferably, the crystal has a ligand associated therewith.

Preferably, the ligand binding domain comprises amino acid residues selected from one or more of the following amino acid residues: I255, P253, A252, Y250, P234, P129, G127, L126, Q125, L47, V45, H44, W227 and W364.

### X-Ray Structures

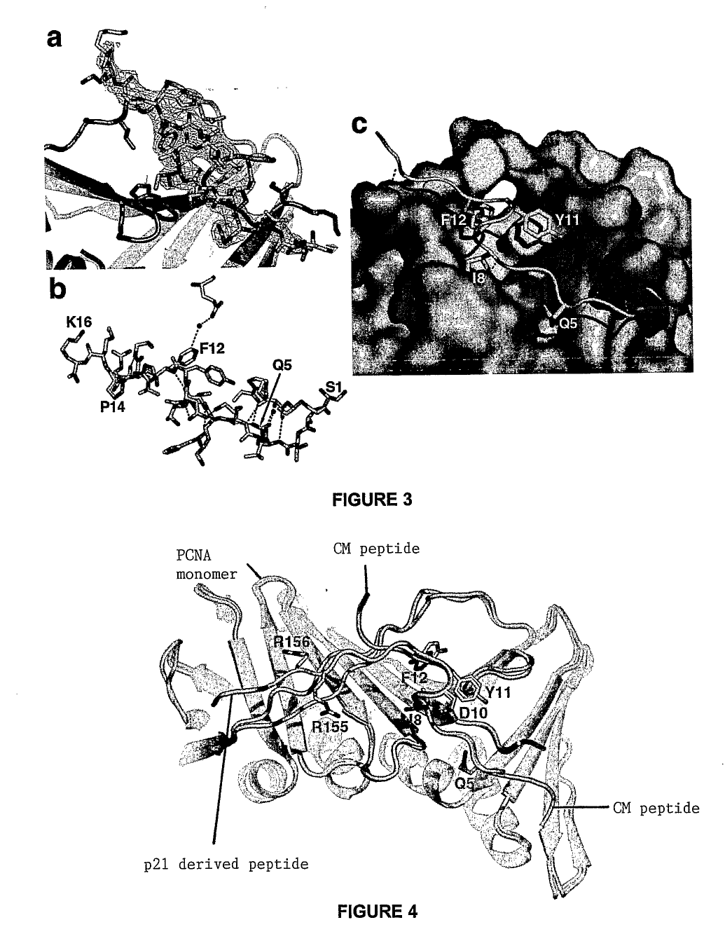

Details of data collection and refinement for the X-ray structures of the CM peptide-PCNA complex and the two free forms of human PCNA are given in Table 1. Together they provide structural information on 11 crystallographically independent PCNA molecules. The PCNA-CM peptide complex structure (FIG. 2) has two independent trimers in the asymmetric unit with each of the six molecules showing clear electron density for all of the complete 16mer CM peptide ligands (FIG. 3a). In each of the six molecules interactions and peptide binding modes are similar. The CM peptide only interacts with one of the PCNA chains in the trimer and is held in place by a total of 6 main chain to backbone H-bonds, as well as by a further two H-bonds involving side-chain atoms (FIG. 3b). The N-terminal residues of the CM peptide form a short stretch of antiparallel sheet with the C-terminal residues of PCNA; the H-bonds L4(N)-I255(O) and L4(O)-I255(N) are conserved in most of the six copies of the complex. The C-terminal residues from 255 to 261 are not visible in the free PCNA structures and the ligand must thus play a role in tying down this disorder. A similar effect was noted in the complex of A. fulgidus PCNA with a 12mer FEN-1 peptide, in which a short β-sheet is formed with the C-terminal PCNA residues, providing a putative control mechanism for mismatch repair³². A prominent feature of the PCNA molecule is the linker strand, comprising residues 121-132, which tethers together the N— and C-terminal domains (FIG. 2), and which was shown to be important in the recognition of p21¹¹. The N-terminal end of this strand is involved in binding to the CM peptide with the formation of two H-bonds: H13(O)-G127(N) and K15(N)-Q125(O) conserved in all six copies of the complex; resulting in a rigidification of this region of the molecule. The overall binding picture (FIG. 3c) shows the N— and C-terminal ends of the CM peptide in a rather extended conformation, forming pairs of H-bonds with the rather mobile C-terminus and the linker strand, while the central helical residues of CM-peptide fit into a more ordered grooved surface and are held in place by only an additional three H-bonds: Q5(NE2)-A252(O), K6(N)-P253(O), and I8(N)-H44(O). It is notable that most of the H-bonds between the CM peptide and PCNA are between backbone donors and acceptors; the involvement of the glutamine side chain being an important exception.

The two native PCNA crystal structures (Table 1) offer the first published information on the conformations of free human PCNA. These crystals belong to space groups C2 and P3 and together they contain a total of five crystallographically independent PCNA monomer structures: the C2 form has one trimer in the asymmetric unit, whereas the P3 form has two independent (crystallographically exact) trimers in the unit cell. A root-mean-square (RMS) fit of 0.16 Å for the P3 trimer Cα atoms shows that they are essentially identical. The overall fit of the P3 and C2 trimer rings, however, shows a buckling of the C2 trimer compared with the crystallographically constrained planar rings in the P3 structure: a fit of Cα atoms from any one subunit of the C2 structure onto the P3 structure, excluding the two regions of high mobility (residues 115-133 and the protruding loop 184-194), gives a RMS fit for the 218 Cα atoms of around 0.7 Å. However, the average RMS fit of Cα atoms of the other two subunits are near 1 Å and 2 Å, respectively. The individual atomic anisotropic temperature factors for each of the 11 PCNA monomers show a very similar pattern. Regions with high temperature factors correspond to exposed flexible loops on the PCNA surface (residue numbers 62-66, 92-95, 120-130, 161-165, and 184-189). The distribution of high B-factors associated with the flexible loops is always similar and is independent of the three different crystal packing arrangements presented here, indicating that the loop regions are inherently flexible. The conserved flexible regions are highlighted in FIG. 2b The only break in the mobile perimeter surface of the free trimer is at the ‘cold’ interface between the subunits. Ligand binding serves to lower temperature factors and rigidify the linker-strand (121-132) and the C-terminal tail (residues 251-261).

Despite substantial differences in amino acid composition, the N-terminal octapeptide helical sequence of CM peptide and the PCNA-bound 22mer fragment of p21¹¹ show similar conformations and interactions with PCNA (FIG. 4). The conserved glutamine of the QXXhXXaa PCNA-recognition motif plays critical roles both in determining peptide conformation (by forming the intramolecular H-bond Q5(NE2)-K6(O)) and also in recognition and binding of PCNA by forming two intermolecular H-bonds (FIG. 3b). The glutamine side chain refines to a similar positions in both the p21 complex¹¹ and the CM peptide complex (FIG. 4). At the base of a rather hydrophobic pocket formed by L251, A252, and F207, a H-bond is formed between the carbonyl oxygen of A252 and the amide nitrogen of the glutamine side chain; a structural water molecule anchors the carbonyl oxygen of the side chain with a second H-bond. The extended conformations of the bound peptides change at residue K7 (FIG. 4) and residues I8-T9-D10-Y11 adopt a 3₁₀-helical conformation, which is stabilised by three intra-molecular H-bonds; K7(O)-D10(N)=2.8 Å, I8(O)-Y11(N)=2.9 Å and I8(O)-F12(N)=2.7 Å. The conformation of the D10 side chain is held in place by a conserved intra-residue D10(OD2)-D10(N) H-bond. There is also a favourable electrostatic interaction with the CM peptide side chain amine of K6. While D10 makes no PCNA contacts, the presence of these intramolecular contacts are nonetheless influential on peptide inhibition as shown by the marked decrease in activity when replaced by alanine or serine²⁸. This suggests that D10 (along with Q5) is important in stabilising the bound helical conformation of the peptide ligands. The local helical conformation places the side chain of I8 into a deep hydrophobic pocket formed by P234, L126, M40, and H44 (FIG. 3c). On the same side of the helix, F12 fits into an extension of the same deeply grooved binding pocket, which is formed by the flexible strand (residues 122-132) wrapping across an anti-parallel sheet (principally involving residues 232-236, 249-253, and 45-49).

The conformations of the bound CM and p21 peptide structures diverge significantly at the C-terminal end of the helix; H13 (H152) adopt different conformations in the two complexes, with the side chains pointing in different directions. The C-terminal 8 residues of the p21 peptide fold down onto the extended linker residues (L126 to M129), forming a stretch of antiparallel β-sheet. In contrast the PKK terminal residues of the CM peptide straddle the linker strand with P14 providing a bridge clamped by two main chain H-bonds H13(O)-G127(N) and K15(N)-Q125(O). The conformation of the linker sequence (residues 119-134) is clearly different in the p21 22mer and CM 16mer peptide complexes (FIG. 4).

### 20Mer Peptide Mimics the Assembly Role of Full-Length P21

p21 interacts with both cyclin/CDK complexes and also with PCNA and the results presented here suggest that a region of p21 can act like double-sided tape to glue both cyclin/CDK and PCNA together to form a quaternary complex. It has previously been shown that both the CM peptide and a C-terminal p21 peptide (residues 141-160) bind PCNA with nanomolar affinity²⁸. Furthermore, the CM peptide inhibits in vitro SV40 DNA replication with a potency very similar to that of p21(141-160). However, whereas the CM peptide is devoid of CDK/cyclin inhibitory activity, the p21(141-160) peptide inhibits CDK4/cyclin D1 kinase activity with an IC₅₀ of 24 μM (FIG. 6a). We hypothesized that due to competitive binding, addition of PCNA to kinase assay mixtures should reverse the inhibitory effect of the p21 peptide. Surprisingly, addition of 10 μM PCNA to kinase assays containing 1 μM CDK4/cyclin D1 did not change significantly the effect of the p21 peptide on pRb phosphorylation (FIG. 6). In fact PCNA concentrations up to 15 μM did not diminish the CDK4-inhibitory potency of p21(141-160), although higher concentrations of PCNA did result in decreased pRb phosphorylation (FIG. 6b). The K_{d} values of binding of p21 peptide to PCNA and CDK4/cyclin are 80 nM and 24,000 nM, respectively, which means that with the concentrations of PCNA, p21-peptide, and CDK4/cyclin used in the assay, up to 50% of the available p21 peptide should be bound to PCNA. The loss of this bound peptide would give an expected (but unobserved) enhancement in the phosphorylation assay of some 20%. One explanation for the lack of effect on addition of PCNA in this concentration range is that the p21(141-160) peptide is capable of binding PCNA and the CDK4/cyclin D1 complex simultaneously while maintaining its inhibitory effect on CDK4/cyclin D1.

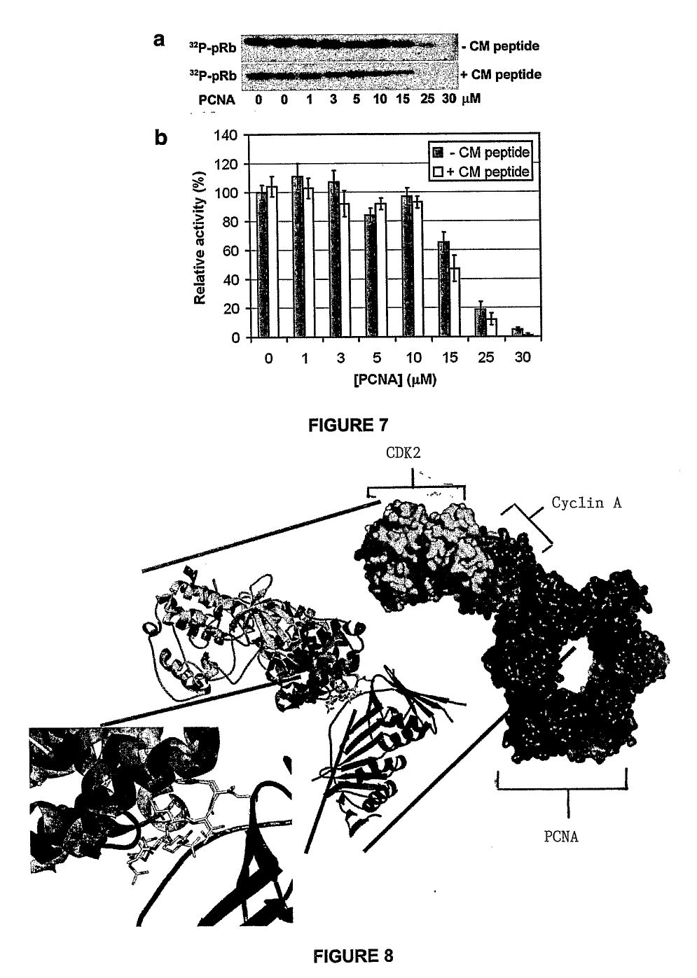

The direct effect of PCNA on CDK4/cyclin D1 kinase activity was studied. At up to 10 μM PCNA the kinase activity of 1 μM CDK4/cyclin D1 was undiminished (FIG. 7). At higher concentrations kinase activity was affected, however, and at 30 μM PCNA the phosphorylation of pRb was almost completely abolished. The IC₅₀ value determined for the inhibition of CDK4/cyclin D1 kinase activity by PCNA was 18±2.4 μM. A possible explanation for the PCNA-induced inhibition of CDK4/cyclin D1 kinase activity is direct binding of PCNA to cyclin D1, leading to disruption of the CDK4/cyclin D1 complex when PCNA is present in excess. It has been demonstrated that PCNA forms complexes with D-type cyclins in vitro. Analysis of a set of deletion mutants of PCNA revealed that either the N— (residues 2-64) or the C-terminus (residues 197-228) is necessary for the direct association of PCNA with D-type cyclins, i.e. sites that are distinct from the p21-binding site of PCNA³³. However, it is still controversial whether trimeric PCNA does actually bind to cyclin D in vivo, and even if it did, how an increase of free cyclin D would lead to growth arrest¹³. The in vitro inhibitory effect of high concentrations of PCNA on CDK4/cyclin D1 kinase activity is in any case unlikely to be relevant in vivo as the cellular levels of PCNA are comparatively lower (˜5 μM). These results suggest that the increase of the p21(141-160)-induced kinase inhibition in the presence of high concentrations of PCNA is due to direct effects of PCNA rather than to a modulation of the peptide inhibitory activity (FIG. 6b). In the presence of 25 μM CM peptide, which binds to PCNA at the same site as p21(141-160), but does not directly inhibit CDK function, the inhibition of CDK4/cyclin D1 function by PCNA was practically unchanged (IC₅₀=15±1.8 μM) (FIG. 7). This suggests that there is a second, (lower affinity) cyclin D1 binding site which is distinct from the p21-dependent site.

### Model for Quaternary PCNA/P21/Cyclin/CDK Complexes

X-ray crystal structures of CDK/cyclin complexes with inhibitory peptides bound in the so-called cyclin binding groove are known²⁷. The cyclin binding motif present in substrates and other protein partners of A-, D-, and E-type cyclin/CDK complexes has been defined as the sequence ZRXLYY′, where Z and X are basic residues, and Y and Y′ are hydrophobic³⁴. The minimal sequence in p21 that binds cyclins is ¹⁵⁵RRLIF¹⁵⁹. Intriguingly, this pentapeptide corresponds to the C-terminal extension in the PCNA/p21 fragment complex that forms an exposed antiparallel sheet with a section of the interdomain linker (PCNA residues 122-132). As discussed above, the CM peptide does not inhibit CDK function, whereas the p21 peptide with the additional 5 amino acid cyclin-binding motif sequence is an effective inhibitor. It is also striking that the conformation of the RRLIF sequence in the PCNA X-ray structure¹¹ is very similar to that found in our CDK2/cyclin A/RRLIF peptide complex. Furthermore, the side chains protrude in a way that allows the p21 peptide to act like double-sided sticky tape, with one face forming contacts with PCNA and the other face forming complementary contacts with the cyclin groove. It was possible to simply combine the two available X-ray structures and overlay the backbone of the RRLIF structure to give the large quaternary complex of PCNA/p21/CDK2/cyclin A shown in FIG. 8.

The RMS fit of the backbone atoms in the RRLIF peptide for this docked structure is less than 0.5 Å. Surprisingly, even without further modelling or refinement, and despite the tight complementary fit between RRLIF and the two proteins, the docked configuration introduces fewer than 10 direct non-bonded contacts under 3.5 Å between PCNA and cyclin; most of theses involve the side chains D283 and I213 on cyclin A, interacting with side chains from N95, D120, D122, and Q125 on PCNA. Restrained molecular mechanics, keeping all main-chain atoms fixed, was used to optimize side chain contacts. Using this model the calculated buried surface areas for cyclin (in the context of RRLIF and PCNA) and PCNA (in the context of RRLIF and cyclin) are 440 Å² and 340 Å², respectively, giving a total buried surface of 780 Å². The total buried surface calculated using a similar model of cyclin docked onto PCNA, but without the RRLIF peptide present, is only 270 Å², which would be insufficient for stable complex formation. This suggests that for this docking site on PCNA the p21 peptide can act as a genuine adaptor molecule and would be required before cyclin/CDK could be recruited to the PCNA complex.

### Binding Pocket of PCNA

Initial binding experiments showed that it was not possible to truncate either terminus of the p21(141-160) peptide without significant loss of affinity and it was only the use of different amino acid sequences, less related to p21, that led to shorter peptides with appreciable binding affinity to PCNA²⁸. Without the benefit of the CM peptide structure presented here, it was not obvious which features of the PCNA interaction are most important for the binding. In particular, it was not clear whether the contacts formed by the 9 C-terminal peptide residues ¹⁵²HSKRRLIFS¹⁶⁰ in the p21 peptide/PCNA complex¹¹ played a key role. The present studies show that this interaction, along with the short- and long-range electrostatic interactions involving ¹⁵⁵RR¹⁵⁶ and ¹⁴⁰RKRR¹⁴³ at the C and N-terminal ends of the peptide, do not seem to be a requirement. These are important considerations in terms of drug design, since incorporation into peptidomimetics of groups capable of multiple Coulombic interactions—so important in protein-protein interactions—does not usually permit the development of permeable and drug-like small molecules.

The solvent-accessible surface on PCNA that is buried when a complex is formed with p21(139-160) has an area of 960 Å². This compares with a buried surface area of 680 Å² on complex formation with CM-peptide. The X-ray structures show that the p21(139-160) complex forms some 15 direct H-bonds with PCNA. This compares with only 8 direct H-bonds between the CM peptide and PCNA. Despite these differences the CM peptide has comparable affinity for PCNA (K_{d}=100 nM) compared with that of a closely related p21 peptide (residues 141-160) (K_{d}=88 nM)²⁸. Isothermal calorimetry measurements²⁸ show that the enthalpy of binding is only 2% more favourable for p21(141-160) (−9758 kcal/mol) compared with CM-peptide (−9518 kcal/mol) while the entropic contribution is significantly less favourable (0.09 cal mol⁻¹ ° C.⁻¹ compared with 0.61 cal mol⁻¹ ° C.⁻¹ for the CM-peptide). This suggests that the additional RRLIFS C-terminal residues (p21(155-160)) that bind to the linker strand do not contribute significantly to the net enthalpy of binding. Furthermore the less favourable entropic contribution to binding for p21(141-160) suggests that an entropic penalty must be paid when this longer, flexible tail binds to the linker strand. It is reasonable to argue that the unfavourable entropic term can be reduced if the peptide is pre-organised in the appropriate binding conformation. There is good evidence that this occurs in the helical region of the peptide from isothermal calorimetry experiments for two peptides: p21(141-160) and the site point mutant p21(141-160)D149A²⁸. The structures of both CM-peptide and p21(139-160) (FIG. 3) show that the aspartate side chain of D10 (or D149) can form two intramolecular H-bonds that may help stabilise the 3₁₀-helical conformation. For the D149A mutant the isothermal calorimetry results show an unfavourable entropy term with a drop in Kₐ from 1.1×10⁷ for p21(141-160) down to 7.8×10⁵ for p21D149A. The X-ray structures suggest that this must be a purely entropic effect as there is no direct contact between the aspartate side chain and PCNA. The long-range calculated electrostatic binding energy contribution is also negative (see below) confirming that the major role for D149 is to hold the peptide in a favoured binding conformation.

Inspection of the CM-peptide binding pocket shows that the major interactions with PCNA involve the side chains Q5, I8, Y11 and F12 (FIG. 3c). Together these amino acids contribute 450 Å² or 65% of the total buried surface for the CM-peptide. To further quantify the peptide PCNA interaction, affinity scores for the individual side chains were calculated using coordinates from the respective X-ray structures (Table 2). These values give a measure of the combined van der Waals, H-bonding, and electrostatic contributions to the binding energy. The total calculated non-bonded intermolecular energies are −469 kcal/mol and 478 kcal/mol for p21 (143-160) and CM peptide, respectively, with positively-charged side chains of both peptides providing the major Coulombic contribution to intermolecular energies. Interestingly, there is a stronger interaction energy of M147 of the p21 peptide compared with the corresponding I8 side chain of the CM peptide and this suggests that further changes in the CM peptide sequence could achieve even better binding, perhaps leading to smaller hybrid peptides.

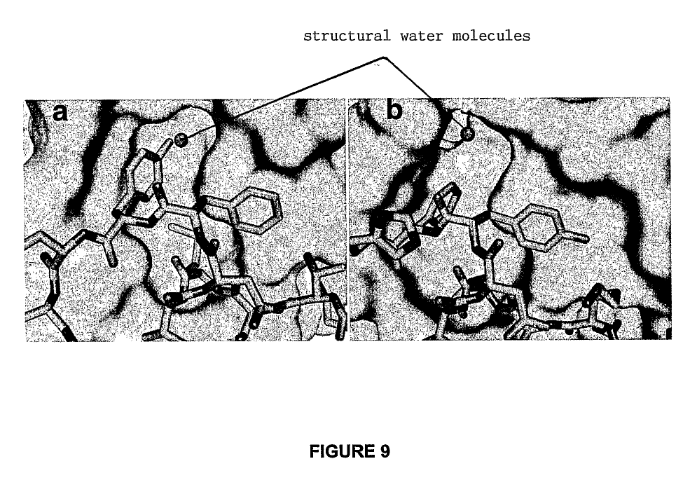

Comparison of the lipophilic pocket (formed in part by the linker strand) of the CM peptide structure with that of p21-bound PCNA reveals some interesting differences. As can be seen (FIG. 9), the overall volume of the CM-peptide pocket is larger and more concave. These changes largely appear to result from movement of the linker-strand to accommodate F12. Of the pocket-forming residues, side-chain atoms from M40, L126, I128, and Q131 all move between 1 and 3 Å relative to the p21 bound structure. Despite the lack of H-bonding contacts of the Y151 hydroxyl with Q131, the induced changes in the pocket result in more effective contact with F12 (see Table 2). Interestingly there is a structural water present in both the CM peptide and the p21(139-160) structures at the base of the F12 (Y151) pocket. The larger hydrophobic volume in the CM peptide structure indicates that bulkier side chains at this position would be accommodated and potentially improve peptide ligand binding. Indeed the deeply grooved and contiguous volume occupied by I8, Y11, and F12 covers approximately 50% of the total buried surface of the CM-peptide and provides a site which looks well suited to the structure-guided design of small-molecule inhibitors of PCNA and the development of novel peptidomimetic drugs.

### PCNA/P21/CDK/Cyclin Complex

In normal, untransformed cells, quaternary complexes are formed between cyclins (A, B, D, and E), CDKs, p21, and PCNA. Furthermore, subunit rearrangement of these CDK complexes is associated with cellular transformation¹⁵. On cell transformation the expression of p21 is frequently depressed and CDKs dissociate from PCNA. This suggests that p21 may participate in the coordination of cellular DNA replication and cell-cycle progression and that upon transformation these processes become uncoupled, permitting escape from the G1 DNA-damage checkpoint. Quaternary complexes of CDK/cyclin pairs with p21 and PCNA exist in multiple cell cycle phases, including G1, S, and even G2/M, where CDK1/cyclin B is implicated³⁵. The quaternary complex (FIG. 8) provides a structural basis to the finding that p21 can both, block access to PCNA for other proteins involved in DNA replication, and also act as an adaptor for various complexes of PCNA with kinases. This model also fits with the suggestion that PCNA acts as an adaptor protein bringing various kinases to substrate proteins involved in DNA replication³⁶.

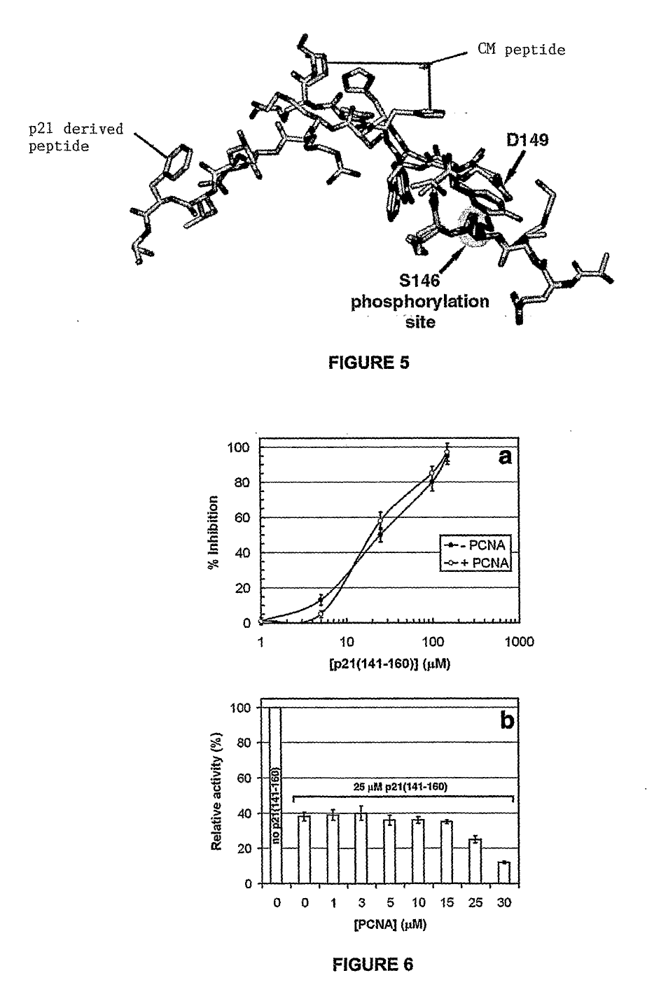

Phosphorylation provides an important and general control mechanism in cell-cycle events. The structures of the PCNA complexes (FIG. 5) provide an explanation of how phosphorylation of p21 can be used to uncouple p21-regulated CDK activity and PCNA-mediated DNA synthesis. Phosphorylation of S146 was shown to prevent the binding of p21 to PCNA in insect cells³⁷ and a subsequent study showed that the protein kinase Akt specifically phosphorylates T145 which abrogates PCNA binding to p21 in endothelial cells. Both of these observations can now be explained as the result of a helix-to-coil conformational switch of p21 induced by the introduction of a phosphate group. Phosphorylation of side chains of either T145 or S146 (FIG. 5) place a bulky negatively charged phosphate group in close proximity (under 4 Å) to the side-chain of D149. The carboxyl group of D149 plays an important role in stabilizing the 3₁₀ helical turn by forming intra-molecular hydrogen bonds. Thus phosphorylation of either T145,S146 or (indeed T148) will prevent the side chain of D149 adopting a conformation promoting the required helical conformation for PCNA binding. This fits well with thermodynamic results from our previous binding studies which showed that residues flanking the helical motif ⁵QKKITDYF¹² along with residue D149 are important for formation and stabilization of the (pre-formed) 3₁₀ helical structure which is required for binding.

### Assays

Another aspect of the invention relates to a method of screening for a ligand capable of binding to a ligand binding domain, wherein said method comprises the use of a crystal as described hereinbefore or the structure co-ordinates of Table 3, Table 4 and/or Table 5.

Another aspect of the invention relates to a method of screening for a ligand capable of binding to a ligand binding domain, wherein the ligand binding domain is as defined hereinabove, the method comprising contacting the ligand binding domain with a test compound and determining if said test compound binds to said ligand binding domain.

Yet another aspect of the invention relates to a method of screening for a modulator of PCNA, wherein the method comprises using a crystal as defined hereinabove, or the structure coordinates of Table 3, Table 4 and/or Table 5.

In one preferred embodiment, the method comprises the steps of:
(a) providing at least a portion of the structure co-ordinates of Table 3, Table 4 and/or Table 5;
(b) employing at least a portion of the structure co-ordinates of Table 3, Table 4 and/or Table 5 to design or select or synthesise a putative modulator of PCNA;
(c) contacting the putative modulator of PCNA with PCNA or a mutant, variant, homologue, derivative or fragment thereof in the presence of a substrate; and
(d) determining whether said putative modulator of PCNA modulates PCNA.

In a preferred embodiment, at least a portion of the structure co-ordinates of Tables 3, 4 and/or 5 and/or the putative modulator of PCNA and/or the substrate are provided on a machine-readable data storage medium comprising a data storage material encoded with machine readable data.

In a preferred embodiment, the putative modulator of PCNA is selected from a library of compounds. Preferably, the library is an in silico library. Suitable in silico libraries will be familiar to those skilled in the art, and include the Available Chemical Directory WL Inc), the Derwent World Drug Index (WDI), BioByteMasterFile, the National Cancer Institute database (NCI), and the Maybridge catalogue.

In another preferred embodiment, the putative modulator of PCNA is selected from a database.

In another preferred embodiment, the putative modulator of PCNA is designed de novo.

In yet another preferred embodiment, the putative modulator of PCNA is designed from a known PCNA modulator.

Preferably, the design or selection of the putative modulator of PCNA is performed in conjunction with computer modelling.

In one particularly preferred embodiment, the putative modulator of PCNA inhibits PCNA activity.

In a further preferred embodiment, the putative modulator of PCNA is useful in the prevention and/or treatment of a PCNA related disorder.

Even more preferably, the PCNA related disorder is a proliferative disorder.

More preferably still, the proliferative disorder is selected from cancer, leukemia, glomerulonephritis, rheumatoid arthritis, psoriasis and chronic obstructive pulmonary disorder.

A further aspect of the invention relates to an assay for a candidate compound capable of modulating PCNA, said assay comprising the steps of:
(a) contacting said candidate compound with PCNA;
(b) detecting whether said candidate compound forms associations with one or more amino acid residues corresponding to PCNA amino acid residues I255, P253, A252, Y250, P234, P129, G127, L126, Q125, L47, V45, H44, W227 and W364.

In one preferred embodiment, said candidate compound is selected by performing rational drug design with a 3-dimensional model of PCNA in conjunction with computer modelling.

Preferably, the assay is a competitive binding assay using a known modulator of PCNA.

Another aspect of the invention relates to a computer for producing a three-dimensional representation of PCNA wherein said computer comprises:
(a) a computer-readable data storage medium comprising a data storage material encoded with computer-readable data, wherein said data comprises the structure co-ordinates of Table 3, Table 4 and/or Table 5;
(b) a working memory for storing instructions for processing said computer-readable data;
(c) a central-processing unit coupled to said working memory and to said computer-readable data storage medium for processing said computer-machine readable data into said three-dimensional representation; and
(d) a display coupled to said central-processing unit for displaying said three-dimensional representation.

Another aspect of the invention relates to a machine-readable data storage medium comprising a data storage material encoded with machine readable data, wherein the data is defined by at least a portion of the structure co-ordinates of Table 3, Table 4 and/or Table 5.

A further aspect of the invention relates to the use of the above-described computer or machine readable data storage medium to predict the structure and/or function of potential modulators of PCNA.

Another aspect of the invention relates to the use of at least a portion of the structure co-ordinates of Table 3, Table 4 and/or Table 5 to screen for modulators of PCNA.

A further aspect of the invention relates to the use of at least a portion of the structure co-ordinates of Table 3, Table 4 and/or Table 5 to solve the structure of the crystalline form of any other protein with significant amino acid sequence homology to any functional domain of PCNA.

Preferably, the structure of the crystalline form of any other protein with significant amino acid sequence homology to any functional domain of PCNA is solved using molecular replacement.

Yet another aspect of the invention relates to the use of at least a portion of the structure co-ordinates of Table 3, Table 4 and/or Table 5 in molecular design techniques to design, select and synthesise modulators of PCNA.

Another aspect of the invention relates to the use of at least a portion of the structure co-ordinates of Table 3, Table 4 and/or Table 5 to screen small molecule databases for chemical entities or compounds that modulate PCNA.

Preferably, the modulator of PCNA, chemical entity, substrate or compound selectively modulates PCNA.

As used throughout, the term “selectively” refers to modulators, ligands or candidate compounds that are selective for PCNA. Preferably, the modulators, ligands or candidate compounds act independently of cyclin groove inhibitors. Preferably the modulators are selective for PCNA over the cyclin binding groove and do not substantially bind to the cyclin binding groove. Preferably the modulators of the invention have a selectivity ratio for PCNA over the cyclin binding groove of greater than 2, more preferably greater than 5, more preferably still greater than 10. Even more preferably, the selectivity ratio for PCNA over the cyclin binding groove is greater than 25, or more preferably still greater than 50 or 100. Selectivity ratios may readily be determined by the skilled person.

In one preferred embodiment, the PCNA modulator, ligand or candidate compound modulates PCNA activity but does not substantially bind to the cyclin binding groove.

In one particularly preferred embodiment, the PCNA modulator, ligand or candidate compound binds substantially exclusively to PCNA.

### PCNA Modulators

A further aspect of the invention relates to a PCNA modulator or ligand identified by the above-described methods, or a candidate compound identified by the above-described assay.

Preferably, the PCNA modulator, ligand or candidate compound of the invention inhibits PCNA activity.

More preferably, the PCNA modulator, ligand or candidate compound of the invention selectively inhibits PCNA.

In one particularly preferred embodiment, the PCNA modulator or candidate compound of the invention is capable of forming associations with one or more amino acid residues corresponding to I255, P253, A252, Y250, P234, P129, G127, L126, Q125, L47, V45, H44, W227 and W364.

Another aspect of the invention relates to a human PCNA ligand binding domain agonist, wherein said ligand binding domain comprises amino acid residues selected from one or more of the following: I255, P253, A252, Y250, P234, P129, G127, L126, Q125, L47, V45, H44, W227 and W364.

Yet another aspect of the invention relates to a human PCNA binding domain antagonist, wherein said ligand binding domain comprises amino acid residues selected from one or more of the following: I255, P253, A252, Y250, P234, P129, G127, L126, Q125, L47, V45, H44, W227 and W364.

The present invention permits the use of molecular design techniques to design, select and synthesise chemical entities and compounds, including PCNA modulating compounds, capable of binding to PCNA, in whole or in part.

By way of example, the structure co-ordinates of Table 3, Table 4 and/or Table 5 may be used to design compounds that bind to PCNA and may alter the physical properties of the compounds (eg. solubility) or PCNA itself. This invention may be used to design compounds that act as modulators, such as competitive inhibitors—of PCNA by binding to all or a portion of the active site of PCNA. Compounds may also be designed that act as non-competitive inhibitors of PCNA. These non-competitive inhibitors may bind to all or a portion of PCNA already bound to its substrate and may be more potent and specific than known PCNA inhibitors that compete only for the PCNA active site. Similarly, non-competitive inhibitors that bind to and inhibit PCNA whether or not it is bound to another chemical entity may be designed using the structure co-ordinates of PCNA described herein.

The present invention may also allow the development of compounds that can isomerise to reaction intermediates in the chemical reaction of a substrate or other compound that binds to PCNA. Thus, the time-dependent analysis of structural changes in PCNA during its interaction with other molecules may be performed. The reaction intermediates of PCNA may also be deduced from the reaction product in co-complex with PCNA. Such information is especially useful to design improved analogues of known PCNA modulators or to design new PCNA modulators based on the reaction intermediates of the PCNA enzyme and PCNA-modulator complex. This may provide a new route for designing PCNA modulators with high specificity and stability. Preferably, this provides a new route for designing PCNA modulators with high specificity, high stability and low toxicity.

Small molecule databases or candidate compounds may be screened for chemical entities or compounds that can bind in whole, or in part, to PCNA. Thus, in a preferred embodiment, the putative PCNA modulator is from a library of compounds or a database. In this screening, the quality of fit of such entities or compounds to the binding site may be judged by various methods—such as shape complementarity or estimated interaction energy (Meng, E. C. et al., J Comp. Chem., 13, pp. 505-524 (1992)).

The structure co-ordinates of Table 3, Table 4 and/or Table 5, or portions thereof, may also be useful in solving the structure of other crystal forms of PCNA. They may also be used to solve the structure of PCNA mutants, PCNA variants, PCNA homologues, PCNA derivatives, PCNA fragments and PCNA complexes.

Preferably, the structure co-ordinates of Table 3, Table 4 and/or Table 5 may be used to solve the structure of the crystalline form of proteins having significant amino acid sequence homology to any functional domain of PCNA. By way of example, molecular replacement may be used. In this method, the unknown crystal structure, whether it is another crystal form of PCNA, a PCNA mutant, a PCNA variant, a PCNA homologue (eg. another protein with significant amino acid sequence homology to any functional domain of PCNA), a PCNA derivative, a PCNA fragment or a PCNA co-complex may be determined using the PCNA structure co-ordinates of the present invention. This method will provide a more accurate structural form for the unknown crystal more quickly and efficiently than attempting to determine such information ab initio.

In a preferred embodiment of the present invention, the PCNA crystal of unknown structure further comprises an entity bound to the PCNA protein or a portion thereof, for example, an entity that is an inhibitor of PCNA.

The crystal structures of such complexes may be solved by molecular replacement or in combination with MAD (Multiwavelength Anomalous Dispersion) and/or MIRAS (Multiple Isomorphous Replacement with Anomalous Scattering) procedures—and compared with that of wild-type PCNA. Potential sites for modification within the binding sites of the enzyme may thus be identified. This information provides an additional tool for determining the most efficient binding interactions, for example, increased hydrophobic interactions, between PCNA and a chemical entity or compound.

The structures and complexes of PCNA may be refined using computer software—such as X-PLOR (Meth. Enzymol., vol. 114 & 115, H. W. Wyckoff et al., eds., Academic Press (1985)), MLPHARE (Collaborative computational project Number 4. The CCP4 Suite: Programs for Protein Crystallography (1994) Acta Crystallogr. D 50, 760-763) and SHARP [De La Fortelle, E. & Bricogne, G. Maximum-likelihood heavy-atom parameters refinement in the MIR and MAD methods (1997) Methods Enzymol. 276, 472-494). Preferably, the complexes are refined using the program CNS (Brünger et al. (1998) Acta Crystallogr. D 54, 905-921). During the final stages of refinement water molecules, ions and inhibitor molecules may be inserted in the structure. This information may thus be used to optimise known classes of PCNA modulators, eg. PCNA inhibitors, and more importantly, to design and synthesise novel classes of PCNA modulators.

The overall figure of merit may be improved by iterative solvent flattening, phase combination and phase extension with the program SOLOMON [Abrahams, J. P. & Leslie, A. G. W. Methods used in structure determination of bovine mitochondrial F1 ATPase. (1996) Acta Crystallogr. D 52, 110-119].

The structure co-ordinates of Table 3, Table 4 and/or Table 5 may also facilitate the identification of related proteins or enzymes analogous to PCNA in function, structure or both, thereby further leading to novel therapeutic modes for treating or preventing PCNA related diseases.

The design of compounds that bind to or modulate PCNA according to the present invention generally involves consideration of two factors. Firstly, the compound must be capable of physically and structurally associating with PCNA. Non-covalent molecular interactions important in the association of PCNA with its substrate may include hydrogen bonding, van der Waals and hydrophobic interactions. Secondly, the compound must be able to assume a conformation that allows it to associate with PCNA. Although certain portions of the compound may not directly participate in the association with PCNA, those portions may still influence the overall conformation of the molecule. This may have a significant impact on potency. Such conformational requirements include the overall three-dimensional structure and orientation of the chemical entity or compound in relation to all or a portion of a binding site of PCNA, or the spacing between functional groups of a compound comprising several chemical entities that directly interact with PCNA.

The potential modulating or binding effect of a chemical compound on PCNA may be analysed prior to its actual synthesis and testing by the use of computer modelling techniques. If the theoretical structure of the given compound suggests insufficient interaction and association with PCNA, then synthesis and testing of the compound may be obviated. However, if computer modelling indicates a strong interaction, the molecule may be synthesised and tested for its ability to bind to PCNA and modulate (eg. inhibit) using the fluorescent substrate assay of Thornberry et al. (2000) Methods Enzymol. 322, pp 100-110. In this manner, synthesis of inactive compounds may be avoided.

A modulating or other binding compound of PCNA may be computationally evaluated and designed by means of a series of steps in which chemical entities or candidate compounds are screened and selected for their ability to associate with PCNA.

A person skilled in the art may use one of several methods to screen chemical entities or candidate compounds for their ability to associate with PCNA and more particularly with the individual binding sites of PCNA. This process may begin by visual inspection of, for example, the active site on the computer screen based on the PCNA co-ordinates of the present invention. Selected chemical entities or candidate compounds may then be positioned in a variety of orientations, or docked, with PCNA. Docking may be accomplished using software such as Quanta and Sybyl, followed by energy minimisation and molecular dynamics with standard molecular mechanics force fields—such as CHARMM and AMBER.

Specialised computer programs may also assist in the process of selecting chemical entities or candidate compounds. These include but are not limited to MCSS (Miranker and Karplus (1991) Proteins: Structure, Function and Genetics, 11, pp. 29-34); GRID (Goodford (1985) J. Med. Chem., 28, pp. 849-857) and AUTODOCK (Goodsell and Olsen (1990), Proteins: Structure. Function, and Genetics, 8, pp. 195-202.

Once suitable chemical entities or candidate compounds have been selected, they may be assembled into a single compound, such as a PCNA modulator. Assembly may proceed by visual inspection of the relationship of the chemical entities or candidate compounds in relation to the structure co-ordinates of PCNA. This may be followed by manual model building using software—such as Quanta, Sybyl, O, HOOK or CAVEAT [Jones, T. A., Zou, J. Y., Cowan, S. W. & Kjeldgaard, M. Improved methods for building protein models in electron density maps and the location of errors in these models (1991) Acta Crystallogr. A 47, 110-119].

Refinement of the model may be carried out using the program CNS [Brünger, A. T. et al. Crystallography & NMR System: A new software suite for macromolecular structure determination. (1998) Acta Crystallogr. D 54, 905-921].

Various programs may be used by a skilled person to connect the individual chemical entities or candidate compounds, such as 3D Database systems (Martin (1992) J. Med. Chem., 35, pp. 2145-2154) and CAVEAT (Bartlett et al. (1989) Royal Chem. Soc. 78, pp. 182-196).

Rather than build a PCNA inhibitor one chemical entity at a time, modulating or other PCNA binding compounds may be designed as a whole or de novo using either an empty binding site or optionally including some portion(s) of a known inhibitor(s). Such compounds may be designed using programs that may include but are not limited to LEGEND (Nishibata and Itai (1991) Tetrahedron, 47, p. 8985) and LUDI (Bohm (1992) J. Comp. Aid. Molec. Design, 6, pp. 61-78).

Other molecular modelling techniques may also be employed in accordance with this invention—such as those described by Cohen et al., J. Med. Chem., 33, pp. 883-894 (1990); Navia and Mureko (1992) Current Opinions in Structural Biology, 2, pp. 202-210 (1992).

Once a compound has been designed or selected by the above methods, the efficiency with which that compound may bind to PCNA may be computationally evaluated. Specific computer software may be used to evaluate the efficiency of binding (eg. to evaluate compound deformation energy and electrostatic interaction), such as QUANTA/CHARMM (Accelrys Inc., USA) and Insight II/Discover (Biosym Technologies Inc., San Diego, Calif., USA). These programs may be implemented, for instance, using a suitable workstation. Other hardware systems and software packages will be known to those persons skilled in the art.

Once a PCNA-modulating compound has been selected or designed, as described above, substitutions may be made (eg. in atoms or side groups) to improve or modify the binding properties. The substitutions may be conservative ie. the replacement group may have approximately the same size, shape, hydrophobicity and charge as the original group. Such substituted chemical compounds may then be analysed for efficiency of binding to PCNA by the same computer methods described above.

Candidate compounds, ligands and modulators of PCNA etc. which are identified using the methods of the present invention may be screened in assays. Screening can be, for example in vitro, in cell culture, and/or in vivo. Biological screening assays preferably centre on activity-based response models, binding assays (which measure how well a compound binds), and bacterial, yeast and animal cell lines (which measure the biological effect of a compound in a cell). The assays can be automated for high capacity-high throughput screening (HTS) in which large numbers of compounds can be tested to identify compounds with the desired activity.

Current screening technologies are described in Handbook of Drug Screening, edited by Ramakrishna Seethala, Prabhavathi B. Fernandes. New York, N.Y., Marcel Dekker, (2001).

### Modulating PCNA

As herein, the term “modulating” or “modulates” refers to preventing, suppressing, inhibiting, alleviating, restorating, elevating, increasing or otherwise affecting PCNA.

The terms “PCNA modulator” or “modulator of PCNA” are used interchangeably and refer to a single entity or a combination of entities.

The PCNA modulator may be an antagonist or an agonist of PCNA.

As used herein, the term “agonist” means any entity, which is capable of interacting (eg. binding) with PCNA and which is capable of increasing a proportion of the PCNA that is in an active form, resulting in an increased biological response.

As used herein, the term “antagonist” means any entity, which is capable of interacting (eg. binding) with PCNA and which is capable of decreasing (eg. inhibiting) a proportion of the PCNA that is in an active form, resulting in a decreased biological response.

Preferably, the PCNA modulators of the present invention are antagonists of PCNA.

The modulator of PCNA may be an organic compound or other chemical. The modulator of PCNA may be a compound, which is obtainable from or produced by any suitable source, whether natural or artificial. The modulator of PCNA may be an amino acid molecule, a polypeptide, or a chemical derivative thereof, or a combination thereof. The modulator of PCNA may even be a polynucleotide molecule, which may be a sense or an anti-sense molecule. The modulator of PCNA may even be an antibody.

The modulator of PCNA may be designed or obtained from a library of compounds, which may comprise peptides, as well as other compounds, such as small organic molecules.

By way of example, the modulator of PCNA may be a natural substance, a biological macromolecule, or an extract made from biological materials such as bacteria, fungi, or animal (particularly mammalian) cells or tissues, an organic or an inorganic molecule, a synthetic agent, a semi-synthetic agent, a structural or functional mimetic, a peptide, a peptidomimetic, a derivatised agent, a peptide cleaved from a whole protein, or a peptide synthesised synthetically (such as, by way of example, either using a peptide synthesiser or by recombinant techniques or combinations thereof, a recombinant agent, an antibody, a natural or a non-natural agent, a fusion protein or equivalent thereof and mutants, derivatives or combinations thereof).

Typically, the modulator of PCNA will be an organic compound. Typically, the organic compounds will comprise two or more hydrocarbyl groups. Here, the term “hydrocarbyl group” means a group comprising at least C and H and may optionally comprise one or more other suitable substituents. Examples of such substituents may include halo-, alkoxy-, nitro-, an alkyl group, a cyclic group etc. In addition to the possibility of the substituents being a cyclic group, a combination of substituents may form a cyclic group. If the hydrocarbyl group comprises more than one C then those carbons need not necessarily be linked to each other. For example, at least two of the carbons may be linked via a suitable element or group. Thus, the hydrocarbyl group may contain hetero atoms. Suitable hetero atoms will be apparent to those skilled in the art and include, for instance, sulphur, nitrogen and oxygen. For some applications, preferably the modulator of PCNA comprises at least one cyclic group. The cyclic group may be a polycyclic group, such as a non-fused polycyclic group. For some applications, the modulator of PCNA comprises at least the one of said cyclic groups linked to another hydrocarbyl group.

The modulator of PCNA may contain halo groups, for example, fluoro, chloro, bromo or iodo groups, or one or more of alkyl, alkoxy, alkenyl, alkylene and alkenylene groups, each of which may be branched or unbranched.

The modulator of PCNA may be a structurally novel modulator of PCNA, or may be an analogue of a known modulator of PCNA.

Preferably, the PCNA modulators have improved properties over those previously available, for example, fewer side effects.

The modulator of PCNA may be a mimetic, or may be chemically modified.

The modulator of PCNA may be capable of displaying other therapeutic properties.

The modulator of PCNA may be used in combination with one or more other pharmaceutically active agents. If combinations of active agents are administered, then they may be administered simultaneously, separately or sequentially.

### Candidate Compounds

As used herein, the term “candidate compound” includes, but is not limited to, a compound which may be obtainable from or produced by any suitable source, whether natural or not.

The candidate compound may be designed or obtained from a library of compounds, which may comprise peptides, as well as other compounds, such as small organic molecules and particularly new lead compounds. By way of example, the candidate compound may be a natural substance, a biological macromolecule, or an extract made from biological materials—such as bacteria, fungi, or animal (particularly mammalian) cells or tissues, an organic or an inorganic molecule, a synthetic candidate compound, a semi-synthetic candidate compound, a structural or functional mimetic, a peptide, a peptidomimetic, a derivatised candidate compound, a peptide cleaved from a whole protein, or a peptide synthesised synthetically, for example, either using a peptide synthesiser or by recombinant techniques or combinations thereof, a recombinant candidate compound, a natural or a non-natural candidate compound, a fusion protein or equivalent thereof and mutants, derivatives or combinations thereof. The candidate compound may even be a compound that is a modulator of PCNA, such as a known inhibitor of PCNA, that has been modified in some way eg. by recombinant DNA techniques or chemical synthesis techniques.

Typically, the candidate compound will be prepared by recombinant DNA techniques and/or chemical synthesis techniques.

Once a candidate compound capable of interacting PCNA has been identified, further steps may be carried out to select and/or to modify the candidate compounds and/or to modify existing compounds, such that they are able to modulate PCNA.

In one aspect, the modulator of PCNA may act as a model (for example, a template) for the development of other compounds.

A further aspect relates to the use of candidate compounds or PCNA modulators identified by the assays and methods of the invention in one or more model systems, for example, in a biological model, a disease model, or a model for PCNA inhibition. Such models may be used for research purposes and for elucidating further details of the biological, physicochemical, pharmacological and/or pharmacokinetic activity of a particular candidate compound. By way of example, the candidate compounds or PCNA modulators of the present invention may be used in biological models or systems in which the cell cycle is known to be of particular significance, e.g. in models relating to cell fertilization, especially in animals.

### Mimetic

As used herein, the term “mimetic” relates to any chemical which includes, but is not limited to, a peptide, polypeptide, antibody or other organic chemical which has the same qualitative activity or effect as a known compound. That is, the mimetic is a functional equivalent of a known compound.

### Chemical Synthesis Methods

Preferably, the modulator of PCNA of the present invention may be prepared by chemical synthesis techniques.

It will be apparent to those skilled in the art that sensitive functional groups may need to be protected and deprotected during synthesis of a compound of the invention. This may be achieved by conventional techniques, for example as described in “Protective Groups in Organic Synthesis” by T W Greene and P G M Wuts, John Wiley and Sons Inc. (1991), and by P. J. Kocienski, in “Protecting Groups”, Georg Thieme Verlag (1994).

It is possible during some of the reactions that any stereocentres present could, under certain conditions, be racemised, for example if a base is used in a reaction with a substrate having an having an optical centre comprising a base-sensitive group. This is possible during e.g. a guanylation step. It should be possible to circumvent potential problems such as this by choice of reaction sequence, conditions, reagents, protection/deprotection regimes, etc. as is well-known in the art. The compounds and salts may be separated and purified by conventional methods.

Separation of diastereomers may be achieved by conventional techniques, e.g. by fractional crystallisation, chromatography or H.P.L.C. of a stereoisomeric mixture of a compounds or suitable salts or derivatives thereof. An individual enantiomer of a compound may also be prepared from a corresponding optically pure intermediate or by resolution, such as by H.P.L.C. of the corresponding racemate using a suitable chiral support or by fractional crystallisation of the diastereomeric salts formed by reaction of the corresponding racemate with a suitably optically active acid or base.

PCNA, modulators of PCNA or variants, homologues, derivatives, fragments or mimetics thereof may be produced using chemical methods to synthesise the PCNA or the modulator of PCNA in whole or in part. For example, a PCNA peptide or a modulator of PCNA that is a peptide can be synthesised by solid phase techniques, cleaved from the resin, and purified by preparative high performance liquid chromatography (e.g., Creighton (1983) Proteins Structures And Molecular Principles, WH Freeman and Co, New York N.Y.). The composition of the synthetic peptides may be confirmed by amino acid analysis or sequencing (e.g., the Edman degradation procedure; Creighton, supra).

Synthesis of peptides (or variants, homologues, derivatives, fragments or mimetics thereof) may be performed using various solid-phase techniques (Roberge J Y et al (1995) Science 269: 202-204) and automated synthesis may be achieved, for example, using the ABI 43 1 A Peptide Synthesizer (Perkin Elmer) in accordance with the instructions provided by the manufacturer. Additionally, the amino acid sequences comprising the modulator of PCNA, may be altered during direct synthesis and/or combined using chemical methods with a sequence from other subunits, or any part thereof, to produce a variant modulator of PCNA.

### Chemical Modification

In one embodiment, the modulator of PCNA may be a chemically modified modulator of PCNA. The chemical modification of a modulator of PCNA may either enhance or reduce interactions between the modulator of PCNA and the target, such as hydrogen bonding interactions, charge interactions, hydrophobic interactions, van der Waals interactions or dipole interactions.

### Process

Another aspect of the invention relates to a process comprising the steps of:
(a) performing the method according to the invention, or an assay according to the invention;
(b) identifying one or more modulators of PCNA; and
(c) preparing a quantity of said one or more PCNA modulators.

A further aspect of the invention relates to a process comprising the steps of:
(a) performing the method according to the invention, or an assay according to the invention;
(b) identifying one or more PCNA modulators; and
(c) preparing a pharmaceutical composition comprising said one or more identified PCNA modulators.

A further aspect relates to a process comprising the steps of:
(a) performing the method according to the invention, or an assay according to the invention;
(b) identifying one or more PCNA modulators;
(c) modifying said one or more PCNA modulators; and
(d) optionally preparing a pharmaceutical composition comprising said one or more PCNA modulators.

### Pharmaceutical Compositions

Another aspect of the invention relates to a pharmaceutical composition comprising a PCNA modulator, ligand or candidate compound of the invention and a pharmaceutically acceptable carrier, diluent, excipient or adjuvant or any combination thereof. Even though the PCNA modulators, ligands or candidate compounds (including their pharmaceutically acceptable salts, esters and pharmaceutically acceptable solvates) can be administered alone, they will generally be administered in admixture with a pharmaceutical carrier, excipient or diluent, particularly for human therapy. The pharmaceutical compositions may be for human or animal usage in human and veterinary medicine.

Examples of such suitable excipients for the various different forms of pharmaceutical compositions described herein may be found in the “Handbook of Pharmaceutical Excipients, 2^{nd} Edition, (1994), Edited by A Wade and P J Weller.

Acceptable carriers or diluents for therapeutic use are well known in the pharmaceutical art, and are described, for example, in Remington's Pharmaceutical Sciences, Mack Publishing Co. (A. R. Gennaro edit. 1985).

Examples of suitable carriers include lactose, starch, glucose, methyl cellulose, magnesium stearate, mannitol, sorbitol and the like. Examples of suitable diluents include ethanol, glycerol and water.

The choice of pharmaceutical carrier, excipient or diluent can be selected with regard to the intended route of administration and standard pharmaceutical practice. The pharmaceutical compositions may comprise as, or in addition to, the carrier, excipient or diluent any suitable binder(s), lubricant(s), suspending agent(s), coating agent(s), solubilising agent(s).

Examples of suitable binders include starch, gelatin, natural sugars such as glucose, anhydrous lactose, free-flow lactose, beta-lactose, corn sweeteners, natural and synthetic gums, such as acacia, tragacanth or sodium alginate, carboxymethyl cellulose and polyethylene glycol.

Examples of suitable lubricants include sodium oleate, sodium stearate, magnesium stearate, sodium benzoate, sodium acetate, sodium chloride and the like.

Preservatives, stabilizers, dyes and even flavoring agents may be provided in the pharmaceutical composition. Examples of preservatives include sodium benzoate, sorbic acid and esters of p-hydroxybenzoic acid: Antioxidants and suspending agents may be also used.

### Salts/Esters

The PCNA modulators, ligands or candidate compounds of the present invention can be present as salts or esters, in particular pharmaceutically acceptable salts or esters.

Pharmaceutically acceptable salts of the PCNA modulators, ligands or candidate compounds of the invention include suitable acid addition or base salts thereof. A review of suitable pharmaceutical salts may be found in Berge et al, J Pharm Sci, 66, 1-19 (1977). Salts are formed, for example with strong inorganic acids such as mineral acids, e.g. sulphuric acid, phosphoric acid or hydrohalic acids; with strong organic carboxylic acids, such as alkanecarboxylic acids of 1 to 4 carbon atoms which are unsubstituted or substituted (e.g., by halogen), such as acetic acid; with saturated or unsaturated dicarboxylic acids, for example oxalic, malonic, succinic, maleic, fumaric, phthalic or tetraphthalic; with hydroxycarboxylic acids, for example ascorbic, glycolic, lactic, malic, tartaric or citric acid; with aminoacids, for example aspartic or glutamic acid; with benzoic acid; or with organic sulfonic acids, such as (C₁-C₄)-alkyl- or aryl-sulfonic acids which are unsubstituted or substituted (for example, by a halogen) such as methane- or p-toluene sulfonic acid.

Esters are formed either using organic acids or alcohols/hydroxides, depending on the functional group being esterified. Organic acids include carboxylic acids, such as alkanecarboxylic acids of 1 to 12 carbon atoms which are unsubstituted or substituted (e.g., by halogen), such as acetic acid; with saturated or unsaturated dicarboxylic acid, for example oxalic, malonic, succinic, maleic, fumaric, phthalic or tetraphthalic; with hydroxycarboxylic acids, for example ascorbic, glycolic, lactic, malic, tartaric or citric acid; with aminoacids, for example aspartic or glutamic acid; with benzoic acid; or with organic sulfonic acids, such as (C₁-C₄)-alkyl- or aryl-sulfonic acids which are unsubstituted or substituted (for example, by a halogen) such as methane- or p-toluene sulfonic acid. Suitable hydroxides include inorganic hydroxides, such as sodium hydroxide, potassium hydroxide, calcium hydroxide, aluminium hydroxide. Alcohols include alkanealcohols of 1-12 carbon atoms which may be unsubstituted or substituted, e.g. by a halogen).

### Enantiomers/Tautomers

In all aspects of the present invention previously discussed, the invention includes, where appropriate all enantiomers and tautomers of the PCNA modulators, ligands or candidate compounds of the invention. The man skilled in the art will recognise compounds that possess an optical properties (one or more chiral carbon atoms) or tautomeric characteristics. The corresponding enantiomers and/or tautomers may be isolated/prepared by methods known in the art.

### Stereo and Geometric Isomers

Some of the PCNA modulators, ligands or candidate compounds of the invention may exist as stereoisomers and/or geometric isomers, e.g. they may possess one or more asymmetric and/or geometric centres and so may exist in two or more stereoisomeric and/or geometric forms. The present invention contemplates the use of all the individual stereoisomers and geometric isomers of those agents, and mixtures thereof. The terms used in the claims encompass these forms, provided said forms retain the appropriate functional activity (though not necessarily to the same degree).

The present invention also includes all suitable isotopic variations of the PCNA modulators. ligands or candidate compounds, or pharmaceutically acceptable salts thereof. An isotopic variation of a PCNA modulator, ligand or candidate compound of the present invention or a pharmaceutically acceptable salt thereof is defined as one in which at least one atom is replaced by an atom having the same atomic number but an atomic mass different from the atomic mass usually found in nature. Examples of isotopes that can be incorporated into the agent and pharmaceutically acceptable salts thereof include isotopes of hydrogen, carbon, nitrogen, oxygen, phosphorus, sulphur, fluorine and chlorine such as ²H, ³H, ¹³C, ¹⁴C, ¹⁵N, ¹⁷O, ¹⁸O, ³¹P, ³²P, ³⁵S, ¹⁸F and ³⁶Cl, respectively. Certain isotopic variations of the agents and pharmaceutically acceptable salts thereof, for example, those in which a radioactive isotope such as ³H or ¹⁴C is incorporated, are useful in drug and/or substrate tissue distribution studies. Tritiated, i.e., ³H, and carbon-14, i.e., ¹⁴C, isotopes are particularly preferred for their ease of preparation and detectability. Further, substitution with isotopes such as deuterium, i.e., ²H, may afford certain therapeutic advantages resulting from greater metabolic stability, for example, increased in vivo half-life or reduced dosage requirements and hence may be preferred in some circumstances. Isotopic variations of the PCNA modulators, ligands or candidate compounds of the present invention can generally be prepared by conventional procedures using appropriate isotopic variations of suitable reagents.

### Solvates

The present invention also includes solvate forms of the PCNA modulators, ligands or candidate compounds. The terms used in the claims encompass these forms.

### Polymorphs

The invention furthermore relates to PCNA modulators, ligands or candidate compounds of the present invention in their various crystalline forms, polymorphic forms and (an)hydrous forms. It is well established within the pharmaceutical industry that chemical compounds may be isolated in any of such forms by slightly varying the method of purification and or isolation form the solvents used in the synthetic preparation of such compounds.

### Prodrugs

The invention further includes PCNA modulators, ligands or candidate compounds of the present invention in prodrug form. Such prodrugs are generally compounds of the invention wherein one or more appropriate groups have been modified such that the modification may be reversed upon administration to a human or mammalian subject. Such reversion is usually performed by an enzyme naturally present in such subject, though it is possible for a second agent to be administered together with such a prodrug in order to perform the reversion in vivo. Examples of such modifications include ester (for example, any of those described above), wherein the reversion may be carried out be an esterase etc. Other such systems will be well known to those skilled in the art.

### Therapeutic Use

Certain PCNA modulators, ligands or candidate compounds of the present invention have been found to possess anti-proliferative activity and are therefore believed to be of use in the treatment of proliferative disorders, such as cancers, leukaemias or other disorders associated with uncontrolled cellular proliferation such as psoriasis and restenosis.

One aspect of the invention therefore relates to a method of preventing and/or treating a PCNA related disorder comprising administering a PCNA modulator, ligand or candidate compound of the invention and/or a pharmaceutical composition according to the invention, wherein said PCNA modulator, said ligand, said candidate compound or said pharmaceutical, is capable of causing a beneficial preventative and/or therapeutic effect.

A further aspect of the invention relates to the use of a PCNA modulator, ligand or candidate compound according to the invention in the preparation of a medicament for treating a PCNA related disorder. Preferably, the PCNA related disorder is a proliferative disorder, more preferably cancer.

As used herein the phrase “preparation of a medicament” includes the use of the compound directly as the medicament in addition to its use in a screening programme for further therapeutic agents or in any stage of the manufacture of such a medicament.

Preferably, the PCNA dependent disorder is a disorder associated with increased PCNA activity. Even more preferably, the disorder is cancer.

The term “proliferative disorder” is used herein in a broad sense to include any disorder that requires control of the cell cycle, for example cardiovascular disorders such as restenosis and cardiomyopathy, auto-immune disorders such as glomerulonephritis and rheumatoid arthritis, dermatological disorders such as psoriasis, anti-inflammatory, anti-fungal, antiparasitic disorders such as malaria, emphysema and alopecia. In these disorders, the compounds of the present invention may induce apoptosis or maintain stasis within the desired cells as required.

Preferably, the proliferative disorder is a cancer or leukaemia.

In another preferred embodiment, the proliferative disorder is psoriasis.

The compounds of the invention may inhibit any of the steps or stages in the cell cycle, for example, formation of the nuclear envelope, exit from the quiescent phase of the cell cycle (G0), G1 progression, chromosome decondensation, nuclear envelope breakdown, START, initiation of DNA replication, progression of DNA replication, termination of DNA replication, centrosome duplication, G2 progression, activation of mitotic or meiotic functions, chromosome condensation, centrosome separation, microtubule nucleation, spindle formation and function, interactions with microtubule motor proteins, chromatid separation and segregation, inactivation of mitotic functions, formation of contractile ring, and cytokinesis functions. In particular, the compounds of the invention may influence certain gene functions such as chromatin binding, formation of replication complexes, replication licensing, phosphorylation or other secondary modification activity, proteolytic degradation, microtubule binding, actin binding, septin binding, microtubule organising centre nucleation activity and binding to components of cell cycle signalling pathways.

As defined herein, an anti-proliferative effect within the scope of the present invention may be demonstrated by the ability to inhibit cell proliferation in an in vitro whole cell assay, for example using any of the cell lines A549, HeLa, HT-29, MCF7, Saos-2, CCRF-CEM, HL-60 and K-562, or by showing kinase inhibition in an appropriate assay. These assays, including methods for their performance, will be familiar to the skilled artisan. Using such assays it may be determined whether a compound is anti-proliferative in the context of the present invention.

In one preferred embodiment, the compound of the invention is administered orally. Another aspect of the invention relates to a method of modulating PCNA activity in a cell, said method comprising contacting the cell with a modulator of PCNA as defined above and/or a pharmaceutical composition as defined above.

Preferably, the cell is a cancer cell.

### Administration

The pharmaceutical compositions of the present invention may be adapted for oral, rectal, vaginal, parenteral, intramuscular, intraperitoneal, intraarterial, intrathecal, intrabronchial, subcutaneous, intradermal, intravenous, nasal, buccal or sublingual routes of administration.

For oral administration, particular use is made of compressed tablets, pills, tablets, gellules, drops, and capsules. Preferably, these compositions contain from 1 to 250 mg and more preferably from 10-100 mg, of active ingredient per dose.

Other forms of administration comprise solutions or emulsions which may be injected intravenously, intraarterially, intrathecally, subcutaneously, intradermally, intraperitoneally or intramuscularly, and which are prepared from sterile or sterilisable solutions. The pharmaceutical compositions of the present invention may also be in form of suppositories, pessaries, suspensions, emulsions, lotions, ointments, creams, gels, sprays, solutions or dusting powders.

An alternative means of transdermal administration is by use of a skin patch. For example, the active ingredient can be incorporated into a cream consisting of an aqueous emulsion of polyethylene glycols or liquid paraffin. The active ingredient can also be incorporated, at a concentration of between 1 and 10% by weight, into an ointment consisting of a white wax or white soft paraffin base together with such stabilisers and preservatives as may be required.

Injectable forms may contain between 10-1000 mg, preferably between 10-250 mg, of active ingredient per dose.

Compositions may be formulated in unit dosage form, i.e., in the form of discrete portions containing a unit dose, or a multiple or sub-unit of a unit dose.

### Dosage

A person of ordinary skill in the art can easily determine an appropriate dose of one of the instant compositions to administer to a subject without undue experimentation. Typically, a physician will determine the actual dosage which will be most suitable for an individual patient and it will depend on a variety of factors including the activity of the specific compound employed, the metabolic stability and length of action of that compound, the age, body weight, general health, sex, diet, mode and time of administration, rate of excretion, drug combination, the severity of the particular condition, and the individual undergoing therapy. The dosages disclosed herein are exemplary of the average case. There can of course be individual instances where higher or lower dosage ranges are merited, and such are within the scope of this invention.

Depending upon the need, the agent may be administered at a dose of from 0.01 to 30 mg/kg body weight, such as from 0.1 to 10 mg/kg, more preferably from 0.1 to 1 mg/kg body weight.

In an exemplary embodiment, one or more doses of 10 to 150 mg/day will be administered to the patient for the treatment of malignancy.

### PCNA Fragment

Another aspect of the invention relates to a fragment of PCNA, or a homologue, mutant, or derivative thereof, comprising a ligand binding domain, said ligand binding domain being defined by amino acid residue structural coordinates selected from one or more of the following: I255, P253, A252, Y250, P234, P129, G127, L126, Q125, L47, V45, H44, W227 and W364. As used herein, the term “ligand binding domain (LBD)” means the ligand binding region of PCNA which is responsible for ligand binding. The term “ligand binding domain” also includes a homologue of the ligand binding domain, or a portion thereof.

As used herein, the term “portion thereof” means the structural co-ordinates corresponding to a sufficient number of amino acid residues of the PCNA sequence (or homologue thereof) that are capable of interacting with a candidate compound capable of binding to the LBD. This term includes ligand binding domain amino acid residues having amino acid residues from about 4 Å to about 5 Å of a bound compound or fragment thereof. Thus, for example, the structural co-ordinates provided in the homology model may contain a subset of the amino acid residues in the LBD which may be useful in the modelling and design of compounds that bind to the LBD.

In one preferred embodiment, the fragment of PCNA, or a homologue, mutant or derivative thereof, corresponds to a portion of the structure co-ordinates of Table 3, Table 4 and/or Table 5.

Another aspect of the invention relates to the use of the above-described fragment of PCNA, or a homologue, mutant, or derivative thereof, in an assay or method for identifying candidate compounds capable of modulating PCNA. Suitable assays/methods are identical to those described hereinabove.

### Nucleotide Sequences

As used herein, the term “nucleotide sequence” refers to nucleotide sequences, oligonucleotide sequences, polynucleotide sequences and variants, homologues, fragments and derivatives thereof (such as portions thereof) which comprise the nucleotide sequences encoding PCNA.

The nucleotide sequence may be DNA or RNA of genomic or synthetic or recombinant origin, which may be double-stranded or single-stranded whether representing the sense or antisense strand or combinations thereof.

Preferably, the term nucleotide sequence is prepared by use of recombinant DNA techniques (e.g. recombinant DNA). The nucleotide sequences may include within them synthetic or modified nucleotides. A number of different types of modification to oligonucleotides are known in the art. These include methylphosphonate and phosphorothioate backbones, addition of acridine or polylysine chains at the 3′ and/or 5′ ends of the molecule. For the purposes of the present invention, it is to be understood that the nucleotide sequences described herein may be modified by any method available in the art.

It will be understood by a skilled person that numerous different nucleotide sequences can encode the same protein as a result of the degeneracy of the genetic code. In addition, it is to be understood that skilled persons may, using routine techniques, make nucleotide substitutions that do not substantially affect the activity encoded by the nucleotide sequence of the present invention to reflect the codon usage of any particular host organism in which the target is to be expressed. Thus, the terms “variant”, “homologue” or “derivative” in relation to nucleotide sequences include any substitution of, variation of, modification of, replacement of, deletion of or addition of one (or more) nucleic acids from or to the sequence providing the resultant nucleotide sequence encodes a functional protein according to the present invention (or even a modulator of PCNA according to the present invention if said modulator comprises a nucleotide sequence or an amino acid sequence).

### Amino Acid Sequences

As used herein, the term “amino acid sequence” is synonymous with the term “polypeptide” and/or the term “protein”. In some instances, the term “amino acid sequence” is synonymous with the term “peptide”.

The amino acid sequence may be isolated from a suitable source, or it may be made synthetically or it may be prepared by use of recombinant DNA techniques.

### Variants/Homologues/Derivatives/Fragments

The PCNA described herein is intended to include any polypeptide, which has the activity of the naturally occurring PCNA and includes all vertebrate and mammalian forms. Such terms also include polypeptides that differ from naturally occurring forms of PCNA by having amino acid deletions, substitutions, and additions, but which retain the activity of PCNA.

The term “variant” is used to mean a naturally occurring polypeptide or nucleotide sequences which differs from a wild-type or a native sequence.

The term “fragment” indicates that a polypeptide or nucleotide sequence comprises a fraction of a wild-type or a native sequence. It may comprise one or more large contiguous sections of sequence or a plurality of small sections. The sequence may also comprise other elements of sequence, for example, it may be a fusion protein with another protein. Preferably the sequence comprises at least 50%, more preferably at least 65%, more preferably at least 80%, most preferably at least 90% of the wild-type sequence.

The present invention also encompasses the use of variants, homologues and derivatives of nucleotide and amino acid sequences. Here, the term “homologue” means an entity having a certain homology with amino acid sequences or nucleotide sequences. Here, the term “homology” can be equated with “identity”.

In the present context, an homologous sequence is taken to include an amino acid sequence which may be at least 75, 85 or 90% identical, preferably at least 95 or 98% identical to the subject sequence. Although homology can also be considered in terms of similarity (i.e. amino acid residues having similar chemical properties/functions), it is preferred to express homology in terms of sequence identity.

An homologous sequence is taken to include a nucleotide sequence which may be at least 75, 85 or 90% identical, preferably at least 95 or 98% identical to the subject sequence.

Homology comparisons can be conducted by eye, or more usually, with the aid of readily available sequence comparison programs. These commercially available computer programs can calculate % homology between two or more sequences.

% homology may be calculated over contiguous sequences, i.e. one sequence is aligned with the other sequence and each amino acid in one sequence is directly compared with the corresponding amino acid in the other sequence, one residue at a time. This is called an “ungapped” alignment. Typically, such ungapped alignments are performed only over a relatively short number of residues.

Although this is a very simple and consistent method, it fails to take into consideration that, for example, in an otherwise identical pair of sequences, one insertion or deletion will cause the following amino acid residues to be put out of alignment, thus potentially resulting in a large reduction in % homology when a global alignment is performed. Consequently, most sequence comparison methods are designed to produce optimal alignments that take into consideration possible insertions and deletions without penalising unduly the overall homology score. This is achieved by inserting “gaps” in the sequence alignment to try to maximise local homology.

However, these more complex methods assign “gap penalties” to each gap that occurs in the alignment so that, for the same number of identical amino acids, a sequence alignment with as few gaps as possible—reflecting higher relatedness between the two compared sequences—will achieve a higher score than one with many gaps. “Affine gap costs” are typically used that charge a relatively high cost for the existence of a gap and a smaller penalty for each subsequent residue in the gap. This is the most commonly used gap scoring system. High gap penalties will of course produce optimised alignments with fewer gaps. Most alignment programs allow the gap penalties to be modified. However, it is preferred to use the default values when using such software for sequence comparisons. For example when using the GCG Wisconsin Bestfit package the default gap penalty for amino acid sequences is −12 for a gap and 4 for each extension.

Calculation of maximum % homology therefore firstly requires the production of an optimal alignment, taking into consideration gap penalties. A suitable computer program for carrying out such an alignment is the GCG Wisconsin Bestfit package (University of Wisconsin, U.S.A.; Devereux et al., 1984, Nucleic Acids Research 12:387). Examples of other software than can perform sequence comparisons include, but are not limited to, the BLAST package (see Ausubel et al., 1999 ibid—Chapter 18), PASTA (Atschul et al., 1990, J. Mol. Biol., 403-410) and the GENEWORKS suite of comparison tools. Both BLAST and FASTA are available for offline and online searching (see Ausubel et al., 1999 ibid, pages 7-58 to 7-60). However, for some applications, it is preferred to use the GCG Bestfit program. A new tool, called BLAST 2 Sequences is also available for comparing protein and nucleotide sequence (see FEMS Microbiol Lett 1999 174(2): 247-50; FEMS Microbiol Lett 1999 177(1): 187-8)

Although the final % homology can be measured in terms of identity, the alignment process itself is typically not based on an all-or-nothing pair comparison. Instead, a scaled similarity score matrix is generally used that assigns scores to each pairwise comparison based on chemical similarity or evolutionary distance. An example of such a matrix commonly used is the BLOSUM62 matrix—the default matrix for the BLAST suite of programs. GCG Wisconsin programs generally use either the public default values or a custom symbol comparison table if supplied (see user manual for further details). For some applications, it is preferred to use the public default values for the GCG package, or in the case of other software, the default matrix, such as BLOSUM62.

Once the software has produced an optimal alignment, it is possible to calculate % homology, preferably % sequence identity. The software typically does this as part of the sequence comparison and generates a numerical result.

The sequences may also have deletions, insertions or substitutions of amino acid residues, which produce a silent change and result in a functionally equivalent substance. Deliberate amino acid substitutions may be made on the basis of similarity in polarity, charge, solubility, hydrophobicity, hydrophilicity, and/or the amphipathic nature of the residues as long as the secondary binding activity of the substance is retained. For example, negatively charged amino acids include aspartic acid and glutamic acid; positively charged amino acids include lysine and arginine; and amino acids with uncharged polar head groups having similar hydrophilicity values include leucine, isoleucine, valine, glycine, alanine, asparagine, glutamine, serine, threonine, phenylalanine, and tyrosine.

Conservative substitutions may be made, for example according to the Table below. Amino acids in the same block in the second column and preferably in the same line in the third column may be substituted for each other:

| | | |
|---|---|---|
| ALIPHATIC | Non-polar | G A P |
| | | I L V |
| | Polar - uncharged | C S T M |
| | | N Q |
| | Polar - charged | D E |
| | | K R |
| AROMATIC | | H F W Y |

Homologous substitution (substitution and replacement are both used herein to mean the interchange of an existing amino acid residue, with an alternative residue) may occur i.e. like-for-like substitution such as basic for basic, acidic for acidic, polar for polar etc. Non-homologous substitution may also occur i.e. from one class of residue to another or alternatively involving the inclusion of unnatural amino acids such as ornithine (hereinafter referred to as Z), diaminobutyric acid ornithine (hereinafter referred to as B), norleucine ornithine (hereinafter referred to as O), pyriylalanine, thienylalanine, naphthylalanine and phenylglycine.

Replacements may also be made by unnatural amino acids include; alpha* and alpha-disubstituted* amino acids, N-alkyl amino acids*, lactic acid*, halide derivatives of natural amino acids such as trifluorotyrosine*, p-Cl-phenylalanine*, p-Br-phenylalanine*, p-I-phenylalanine*, L-allyl-glycine*, β-alanine*, L-α-amino butyric acid*, L-γ-amino butyric acid*, L-α-amino isobutyric acid*, L-ε-amino caproic acid^{#}, 7-amino heptanoic acid*, L-methionine sulfone^{#}*, L-norleucine*, L-norvaline*, p-nitro-L-phenylalanine*, L-hydroxyproline^{#}, L-thioproline*, methyl derivatives of phenylalanine (Phe) such as 4-methyl-Phe*, pentamethyl-Phe*, L-Phe (4-amino)^{#}, L-Tyr (methyl)*, L-Phe (4-isopropyl)*, L-Tic (1,2,3,4-tetrahydroisoquinoline-3-carboxyl acid)*, L-diaminopropionic acid^{#} and L-Phe (4-benzyl)*. The notation * has been utilised for the purpose of the discussion above (relating to homologous or non-homologous substitution), to indicate the hydrophobic nature of the derivative whereas # has been utilised to indicate the hydrophilic nature of the derivative, #* indicates amphipathic characteristics.

The term “derivative” or “derivatised” as used herein includes chemical modification of an entity, such as candidate compound or a PCNAmodulator. Illustrative of such chemical modifications would be replacement of hydrogen by a halo group, an alkyl group, an acyl group or an amino group.

Variant amino acid sequences may include suitable spacer groups that may be inserted between any two amino acid residues of the sequence including alkyl groups such as methyl, ethyl or propyl groups in addition to amino acid spacers such as glycine or β-alanine residues. A further form of variation, involves the presence of one or more amino acid residues in peptoid form, will be well understood by those skilled in the art. For the avoidance of doubt, “the peptoid form” is used to refer to variant amino acid residues wherein the cc-carbon substituent group is on the residue's nitrogen atom rather than the α-carbon. Processes for preparing peptides in the peptoid form are known in the art, for example Simon R J et al., PNAS (1992) 89(20), 9367-9371 and Horwell D C, Trends Biotechnol. (1995) 13(4), 132-134.

### Mutant

As used herein, the term “mutant” refers to PCNA comprising one or more changes in the wild-type PCNA sequence.

The term “mutant” is not limited to amino acid substitutions of the amino acid residues in PCNA, but also includes deletions or insertions of nucleotides which may result in changes in the amino acid residues in the amino acid sequence of PCNA.

The present invention also enables the solving of the crystal structure of PCNA mutants. More particularly, by virtue of the present invention, the location of the active site of PCNA based on the structural coordinates of Tables 3, 4 and/or 5 permits the identification of desirable sites for mutation. For example, one or more mutations may be directed to a particular site—such as the active site—or combination of sites of PCNA. Similarly, only a location on, at or near the enzyme surface may be replaced, resulting in an altered surface charge of one or more charge units, as compared to the wild-type enzyme. Alternatively, an amino acid residue in PCNA may be chosen for replacement based on its hydrophilic or hydrophobic characteristics.

Such mutants may be characterised by any one of several different properties as compared with wild-type PCNA. For example, such mutants may have altered surface charge of one or more charge units, or have an increased stability to subunit dissociation, or an altered substrate specificity in comparison with, or a higher specific activity than, wild-type PCNA.

The mutants may be prepared in a number of ways that are known by a person skilled in the art. For example, mutations may be introduced by means of oligonucleotide-directed mutagenesis or other conventional methods. Alternatively, mutants of PCNA may be generated by site specific replacement of a particular amino acid with an unnaturally occurring amino acid. This may be achieved by growing a host organism capable of expressing either the wild-type or mutant polypeptide on a growth medium depleted of one or more natural amino acids but enriched in one or more corresponding unnaturally occurring amino acids.

### Host Cells

As used herein, the term “host cell” refers to any cell that comprises nucleotide sequences that are of use in the present invention, for example, nucleotide sequences encoding PCNA.

Host cells may be transformed or transfected with a nucleotide sequence contained in a vector e.g. a cloning vector. Preferably, said nucleotide sequence is carried in a vector for the replication and/or expression of the nucleotide sequence. The cells will be chosen to be compatible with the said vector and may for example be prokaryotic (for example bacterial), fungal, yeast or plant cells.

The gram-negative bacterium E. coli is widely used as a host for cloning nucleotide sequences. This organism is also widely used for heterologous nucleotide sequence expression. However, large amounts of heterologous protein tend to accumulate inside the cell. Subsequent purification of the desired protein from the bulk of E. coli intracellular proteins can sometimes be difficult.

In contrast to E. coli, bacteria from the genus Bacillus are very suitable as heterologous hosts because of their capability to secrete proteins into the culture medium. Other bacteria suitable as hosts are those from the genera Streptomyces and Pseudomonas.

Depending on the nature of the polynucleotide and/or the desirability for further processing of the expressed protein, eukaryotic hosts including yeasts or other fungi may be preferred. In general, yeast cells are preferred over fungal cells because yeast cells are easier to manipulate. However, some proteins are either poorly secreted from the yeast cell, or in some cases are not processed properly (e.g. hyperglycosylation in yeast). In these instances, a different fungal host organism should be selected.

Examples of expression hosts are fungi—such as Aspergillus species (such as those described in EP-A-0184438 and EP-A-0284603) and Trichoderma species; bacteria—such as Bacillus species (such as those described in EP-A-0134048 and EP-A-0253455), Streptomyces species and Pseudomonas species; yeasts—such as Kluyveromyces species (such as those described in EP-A-0096430 and EP-A-0301670) and Saccharomyces species; and mammalian cells—such as CHO-K1 cells.

The PCNA proteins produced by a host recombinant cell may be secreted or may be contained intracellularly depending on the nucleotide sequence and/or the vector used.

The use of host cells may provide for post-translational modifications as may be needed to confer optimal biological activity on recombinant expression products of the present invention.

Aspects of the present invention also relate to host cells comprising the PCNA constructs of the present invention. The PCNA constructs may comprise a nucleotide sequence for replication and expression of the sequence. The cells will be chosen to be compatible with the vector and may for example be prokaryotic (for example bacterial), fungal, yeast or plant cells.

In a preferred embodiment, the host cells are mammalian cells, such as CHO-K1 cells.

### Vector

Aspects of the present invention relate to a vector comprising a nucleotide sequence, such as a nucleotide sequence encoding PCNA or a modulator of PCNA, administered to a subject.

Preferably, PCNA or the modulator of PCNA is prepared and/or delivered using a genetic vector.

As it is well known in the art, a vector is a tool that allows or facilitates the transfer of an entity from one environment to another. In accordance with the present invention, and by way of example, some vectors used in recombinant DNA techniques allow entities, such as a segment of DNA (such as a heterologous DNA segment, such as a heterologous cDNA segment), to be transferred into a host and/or a target cell for the purpose of replicating the vectors comprising nucleotide sequences and/or expressing the proteins encoded by the nucleotide sequences. Examples of vectors used in recombinant DNA techniques include, but are not limited to, plasmids, chromosomes, artificial chromosomes or viruses.

The term “vector” includes expression vectors and/or transformation vectors.

The term “expression vector” means a construct capable of in vivo or in vitrolex vivo expression.

The term “transformation vector” means a construct capable of being transferred from one species to another.

### Regulatory Sequences

In some applications, nucleotide sequences are operably linked to a regulatory sequence which is capable of providing for the expression of the nucleotide sequence, such as by a chosen host cell. By way of example, a vector comprising the PCNA nucleotide sequence is operably linked to such a regulatory sequence i.e. the vector is an expression vector.

The term “operably linked” refers to a juxtaposition wherein the components described are in a relationship permitting them to function in their intended manner. A regulatory sequence “operably linked” to a coding sequence is ligated in such a way that expression of the coding sequence is achieved under conditions compatible with the control sequences.

The term “regulatory sequences” includes promoters and enhancers and other expression regulation signals.

The term “promoter” is used in the normal sense of the art, e.g. an RNA polymerase binding site.

Enhanced expression of a nucleotide sequence, for example, a nucleotide sequence encoding PCNA, may also be achieved by the selection of heterologous regulatory regions, e.g. promoter, secretion leader and terminator regions, which serve to increase expression and, if desired, secretion levels of the protein of interest from the chosen expression host and/or to provide for the inducible control of the expression of PCNA. In eukaryotes, polyadenylation sequences may be operably connected to the PCNA nucleotide sequence.

Preferably, the PCNA nucleotide sequence is operably linked to at least a promoter.

Aside from the promoter native to the gene encoding the PCNA nucleotide sequence, other promoters may be used to direct expression of the PCNA polypeptide. The promoter may be selected for its efficiency in directing the expression of the PCNA nucleotide sequence in the desired expression host.

In another embodiment, a constitutive promoter may be selected to direct the expression of the PCNA nucleotide sequence. Such an expression construct may provide additional advantages since it circumvents the need to culture the expression hosts on a medium containing an inducing substrate.

Hybrid promoters may also be used to improve inducible regulation of the expression construct.

The promoter can additionally include features to ensure or to increase expression in a suitable host. For example, the features can be conserved regions such as a Pribnow Box or a TATA box. The promoter may even contain other sequences to affect (such as to maintain, enhance, decrease) the levels of expression of the PCNAnucleotide sequence. For example, suitable other sequences include the Sh1-intron or an ADH intron. Other sequences include inducible elements—such as temperature, chemical, light or stress inducible elements. Also, suitable elements to enhance transcription or translation may be present.

The PCNA encoding sequence may be fused (eg. ligated) to nucleotide sequences encoding a polypeptide domain which will facilitate purification of soluble proteins (Kroll D J et al (1993) DNA Cell Biol 12:441-53). Preferably, the polypeptide domain which facilitates purification of soluble proteins is fused in frame with the PCNA encoding sequence. Such purification facilitating domains include, but are not limited to, metal chelating peptides—such as histidine-tryptophan modules that allow purification on immobilised metals (Porath J (1992) Protein Expr Purif 3, 263-281), protein A domains that allow purification on immobilised immunoglobulin, and the domain utilised in the FLAGS extension/affinity purification system (Immunex Corp, Seattle, Wash.). The inclusion of a cleavable linker sequence such as Factor XA or enterokinase (Invitrogen, San Diego, Calif.) between the purification domain and PCNA is useful to facilitate purification.

### Expression Vector

Preferably, nucleotide sequences, such as nucleotide sequences encoding PCNA or modulators of PCNA, are inserted into a vector that is operably linked to a control sequence that is capable of providing for the expression of the coding sequence by the host cell.

Nucleotide sequences produced by a host recombinant cell may be secreted or may be contained intracellularly depending on the sequence and/or the vector used. As will be understood by those of skill in the art, expression vectors containing a PCNA encoding nucleotide sequence or a mutant, variant, homologue, derivative or fragment thereof can be designed with signal sequences, which direct secretion of the nucleotide sequence through a particular prokaryotic or eukaryotic cell membrane.

Preferably, the expression vectors are stably expressed in CHO cells as described previously (Ehlers et al. (1996) Biochemistry 35, 9549-9559). More preferably, the expression vectors are pLEN-tACEΔ36g(1, 2, 3, 4) and pLEN-tACEΔ36g(1,3).

### Fusion Proteins

PCNA or a modulator of PCNA may be expressed as a fusion protein to aid extraction and purification and/or delivery of the modulator of PCNA or the PCNA protein to an individual and/or to facilitate the development of a screen for modulators of PCNA.

Examples of fusion protein partners include glutathione-S-transferase (GST), 6xHis, GAL4 (DNA binding and/or transcriptional activation domains) and β-galactosidase.

It may also be convenient to include a proteolytic cleavage site between the fusion protein partner and the protein sequence of interest to allow removal of fusion protein sequences. Preferably, the fusion protein will not hinder the activity of the protein of interest.

The fusion protein may comprise an antigen or an antigenic determinant fused to the substance of the present invention. In this embodiment, the fusion protein may be a non-naturally occurring fusion protein comprising a substance, which may act as an adjuvant in the sense of providing a generalised stimulation of the immune system. The antigen or antigenic determinant may be attached to either the amino or carboxy terminus of the substance.

### Organism

The term “organism” in relation to the present invention includes any organism that could comprise PCNA and/or modulators of PCNA. Examples of organisms may include mammals, fungi, yeast or plants.

Preferably, the organism is a mammal. More preferably, the organism is a human.

### Transformation

As indicated earlier, the host organism can be a prokaryotic or a eukaryotic organism. Examples of suitable prokaryotic hosts include E. coli and Bacillus subtilis. Teachings on the transformation of prokaryotic hosts are well documented in the art, for example see Sambrook et al (Molecular Cloning: A Laboratory Manual, 2nd edition, 1989, Cold Spring Harbor Laboratory Press) and Ausubel et al., Current Protocols in Molecular Biology (1995), John Wiley & Sons, Inc. Examples of suitable eukaryotic hosts include mammalian cells.

If a prokaryotic host is used then the nucleotide sequence, such as the PCNA nucleotide sequence, may need to be suitably modified before transformation—such as by removal of introns.

Thus, the present invention also relates to the transformation of a host cell with a nucleotide sequence, such as PCNA or a modulator of PCNA. Host cells transformed with the nucleotide sequence may be cultured under conditions suitable for the expression and recovery of the encoded protein from cell culture. The protein produced by a recombinant cell may be secreted or may be contained intracellularly depending on the sequence and/or the vector used. As will be understood by those of skill in the art, expression vectors containing coding sequences can be designed with signal sequences which direct secretion of the coding sequences through a particular prokaryotic or eukaryotic cell membrane. Other recombinant constructions may join the coding sequence to nucleotide sequence encoding a polypeptide domain, which will facilitate purification of soluble proteins (Kroll D J et al (1993) DNA Cell Biol 12:441-53) e.g. 6-His or Glutathione-S-transferase.

### Transfection

Vectors comprising for example, the PCNA nucleotide sequence, may be introduced into host cells, for example, mammalian cells, using a variety of methods.

Typical transfection methods include electroporation, DNA biolistics, lipid-mediated transfection, compacted DNA-mediated transfection, liposomes, immunoliposomes, lipofectin, cationic agent-mediated, cationic facial amphiphiles (CFAs) (Nature Biotech. (1996) 14, 556), multivalent cations such as spermine, cationic lipids or polylysine, 1,2,-bis(oleoyloxy)-3-(trimethylammonio)propane (DOTAP)-cholesterol complexes (Wolff and Trubetskoy 1998 Nature Biotechnology 16: 421) and combinations thereof.

Uptake of nucleic acid constructs by mammalian cells is enhanced by several known transfection techniques for example those including the use of transfection agents. Example of these agents include cationic agents (for example calcium phosphate and DEAE-dextran) and lipofectants (for example lipofectam™ and transfectam™). Typically, nucleic acid constructs are mixed with the transfection agent to produce a composition.

Such methods are described in many standard laboratory manuals—such as Sambrook et al., Molecular Cloning: A Laboratory Manual, 2d ed. (1989) Cold Spring Harbor Laboratory Press, Cold Spring Harbor, N.Y.

### SUMMARY

By way of summary, the present invention provides an X-ray crystal structure of PCNA complexed with a 16mer peptide related to p21, which binds with a K_{d} of 100 nM. Two additional crystal structures of native PCNA provide the first structures of free human PCNA and show that the only significant changes on ligand binding involve rigidification of a number of flexible regions on the surface of PCNA. The competitive binding experiments described herein show that a 20mer sequence from p21 can associate simultaneously with PCNA and CDK/cyclin complexes. A structural model for such quaternary complexes is presented, in which the C-terminal sequence of p21 acts as a double-sided tape in that it docks to both the PCNA and cyclin molecules. The quaternary complex shows little direct interaction between PCNA and cyclin, assigning to p21 the role of an adaptor. Taken together, the biochemical and structural results delineate a compact inhibitor site on the surface of PCNA that may be exploited in the design of peptidomimetics, which will act independently of cyclin-groove inhibitors. Blocking this site with drug-like small molecules may be both chemically feasible and therapeutically relevant in proliferative diseases.

The identification of a well-defined ‘druggable’ site on PCNA adjacent to (and potentially independent of) the cyclin binding site identified in the model structure opens up the possibility of using these sites to probe the biological function of PCNA. In particular it will be interesting to study the effects of small and specific ‘cyclin-groove’ inhibitors which are now available²⁷, along with smaller CM-related peptides to examine a possible synergy of action. Such complementary effects could have important consequences in developing inhibitor cocktails to manipulate cell-cycle events.

The present invention is further described by way of example and with reference to the following figures wherein:

FIG. 1 shows the design of consensus motif 1 peptide;

FIG. 2 shows surface features of the PCNA trimer. (a) The three PCNA monomers in the trimer are shown together with the corresponding CM peptides. PCNA residues involved in binding to the CM peptide are highlighted in black. (b) For those residues in the two unliganded structures that show high B-factors (viz. residues 62-66, 93-97, 105-109, 118-132, 161-166, and 184-189), the Cα atoms are depicted as CPK spheres, indicating inherently flexible regions of the molecule: The linker strand (residues 118-132) is a prominent feature.

FIG. 3 shows the PCNA binding pocket for the CM-peptide. (a) Electron density for a 2Fo-Fc map contoured at 1σ for one of the CM peptide ligands. (b) Intra- and intermolecular H-bonds formed by CM-peptide. The 8 conserved intermolecular H-bonds important for recognition of the CM-peptide are shown: L4(N)-I255(O), L4(O)-I255(N), Q5(OE1)-W, Q5(NE2)-A252(O), K6(N)-P253(O), I8(N)-H44(O), H13(O)-G127(N), K15(N)-Q125(O). The 5 conserved intra-molecular H-bonds are also shown: Q5(NE2)-K6(O), K7(O)-D10(N), I8(O)-Y11(N), I8(O)-F12(N), D10(N)-D10(OD1). (c) View of the interactions between the CM peptide (grey ribbon) and PCNA (molecular surface). The 4 residues in the CM peptide making direct contact with PCNA are shown with side chains and are labelled. The PCNA binding pocket is delineated; residues make hydrophobic contacts with I8 and F12 of the CM peptide, whereas others are involved in polar interactions with Y11 and Q5. H-bonds are shown as broken lines.

FIG. 4 shows a comparison of PCNA binding by C-terminal p21 and CM peptides. Alignment of the PCNA structures (one monomer shown only) in complex with the p21-derived peptide ¹³⁹GRKRRQTSMTDFYHSKRRLIFS¹⁶⁰ (PDB #1AXC) and the CM peptide ¹SAVLQKKITDYFHPKK¹⁶. The key interacting residues in the peptides are shown.

FIG. 5 shows p-hosphorylation of p21. Superimposition of the PCNA-bound CM peptide and p21 peptide. Phosporylation of S146 would result in bad contacts with D149.

FIG. 6 shows the effect of PCNA on CDK4-mediated pRb phosphorylation. Inhibition of in vitro pRb phosphorylation by CDK4/cyclin D1 in the presence or absence of 10 μM PCNA (a). The IC₅₀ values were 25±3 μM in the absence and 24±2 μM in the presence of PCNA. PCNA was titrated into the CDK4/cyclin D1 kinase assay reactions in the presence or absence of 25 μM p21(141-160) peptide and relative activities determined (b).

FIG. 7 shows the effect of PCNA on CDK4/cyclin D1 kinase activity in the presence and absence of CM peptide. PCNA was added at different concentrations to the kinase assay reaction mixtures in the presence or absence of 25 μM CM peptide. The reaction mixture was resolved by SDS-PAGE and the autoradiogram showing incorporation of radioactive phosphate into pRb (a) was scanned and quantified using QuickScan software (b).

FIG. 8 shows quaternary PCNA-CDK-cyclin-p21 complex. CDK2, cyclin A, and PCNA are shown. Superimposition of the common RRLIF substructure in the PCNA/p21 peptide (¹³⁹GRKRRQTSMTDFYHSKRRLIFS¹⁶⁰) and our CDK2/cyclin A/p21 peptide ¹⁵⁵RLIF¹⁵⁹ (1OKV) complexes produced the quaternary complex shown. The exploded views show that the RRLIF conformation in the cyclin A- and PCNA-bound cases is practically identical.

FIG. 9 shows a comparison of the binding pockets of PCNA in the p21 (a) and CM (b) peptide structures. Structural water molecules are highlighted.

### EXAMPLES

### General Methods

The methods described here may employ, unless otherwise indicated, conventional techniques of chemistry, molecular biology, microbiology, recombinant DNA and immunology, which are within the capabilities of a person of ordinary skill in the art. Such techniques are explained in the literature. See, for example, J. Sambrook, E. F. Fritsch, and T. Maniatis, 1989, Molecular Cloning: A Laboratory Manual, Second Edition, Books 1-3, Cold Spring Harbor Laboratory Press; Ausubel, F. M. et al. (1995 and periodic supplements; Current Protocols in Molecular Biology, ch. 9, 13, and 16, John Wiley & Sons, New York, N.Y.); B. Roe, J. Crabtree, and A. Kahn, 1996, DNA Isolation and Sequencing: Essential Techniques, John Wiley & Sons; J. M. Polak and James O'D. McGee, 1990, In Situ Hybridization: Principles and Practice; Oxford University Press; M. J. Gait (Editor), 1984, Oligonucleotide Synthesis: A Practical Approach, Irl Press; D. M. J. Lilley and J. E. Dahlberg, 1992, Methods of Enzymology: DNA Structure Part A: Synthesis and Physical Analysis of DNA Methods in Enzymology, Academic Press; Using Antibodies: A Laboratory Manual: Portable Protocol NO. I by Edward Harlow, David Lane, Ed Harlow (1999, Cold Spring Harbor Laboratory Press, ISBN 0-87969-544-7); Antibodies: A Laboratory Manual by Ed Harlow (Editor), David Lane (Editor) (1988, Cold Spring Harbor Laboratory Press, ISBN 0-87969-314-2), 1855. Handbook of Drug Screening, edited by Ramakrishna Seethala, Prabhavathi B. Fernandes (2001, New York, N.Y., Marcel Dekker, ISBN 0-8247-0562-9); and Lab Ref: A Handbook of Recipes, Reagents, and Other Reference Tools for Use at the Bench, Edited Jane Roskams and Linda Rodgers, 2002, Cold Spring Harbor Laboratory, ISBN 0-87969-630-3. Each of these general texts is herein incorporated by reference.

### Materials and Methods

### Production of Recombinant Proteins

PCNA: Recombinant human PCNA was expressed in Escherichia coli BL21(DE3) from a pT7-PCNA expression vector. The protein was purified from the soluble fraction using a four-step chromatographic procedure, including anion exchange (Q-Sepharose, Pharmacia), cation exchange (SP-Sepharose, Pharmacia), hydroxyapatite (BioRad), and size-exclusion (Superose-12, Pharmacia) modes, as described²⁸.

CDK4: The N-terminally His₆-tagged human recombinant protein was expressed in Sf9 insect cells using a baculovirus construct. Sf9 culture (1.6×10⁶ cells/mL) was infected (MOI of 3) for two days. The cells were harvested by low speed centrifugation and the protein was purified from the insect cell pellet by metal affinity chromatography. In brief: the insect cell pellet was lysed in buffer A (10 mM Tris-HCl pH 8.0, 150 mM NaCl, 0.02% Nonidet P40, 5 MM β-mercaptoethanol, 20 mM NaF, 1 mM Na₃VO₄ and Sigma Protease Inhibitor Cocktail) by sonication. The soluble fraction was obtained by centrifugation and was loaded onto Ni-NTA-Agarose (Qiagen). Non-bound protein was removed with 300 mM NaCl, 5-15 mM imidazole in buffer A, and bound protein was eluted with buffer A, supplemented with 250 mM imidazole. The purified protein was dialyzed extensively against storage buffer (20 mM HEPES pH 7.4, 50 mM NaCl, 2 mM DTT, 1 mM EDTA, 1 mM EGTA, 0.02% Nonidet P40, 10% v/v glycerol) and stored at −70° C.

Cyclin D1: Recombinant human cyclin D1 was expressed in E. coli BL21 (DE3) using a PET expression vector. BL21 (DE3) was grown at 37° C. with shaking (200 rpm) to mid-log phase (OD₆₀₀=0.6 AU). Expression was induced by addition of 1 mM IPTG and the culture was incubated for a further 3 h. The bacteria were then harvested by centrifugation, and the cell pellet was re-suspended in 50 mM Tris-HCl pH 7.5, 10% sucrose. Cyclin D1 was purified from the inclusion bodies. The bacterial cells were lysed by treatment with lysozyme and sonication. The insoluble fraction was pelleted by centrifugation. The inclusion bodies were purified by repetitive washing of the insoluble fraction with 50 mM Tris-HCl pH 8.0, 2 mM EDTA, 100 mM NaCl, and 0.5% Triton. Purified inclusion bodies were solubilized with the same buffer, containing 6 M urea. The protein was refolded by slow dilution with 25 mM Tris HCl pH 8.0, 100 mM NaCl, 2 mM DTT, 1 mM EDTA, 0.2% Nonidet P40. After concentration by ultrafiltration (Amicon concentration unit) the protein was further purified by size-exclusion HPLC (Superose 12, Pharmacia).

GST-pRb(773-928): The hyperphosphorylation domain of pRb (residues 773-982) with an N-terminal GST tag was expressed in E. coli BL21(DE3) and was purified on a glutathione-Sepharose column (Pharmacia) according to the manufacturer's instructions. For the 96-well format in vitro kinase assay GST-pRb was used immobilized on glutathione-Sepharose beads.

CDK4/cyclin D1 kinase assay: The reaction mixture consisted of 1 μM of CDK4 and cyclin D1, 5 μg GST-pRb, 100 mM ATP and 0.2 μCi ³²P-ATP. The kinase reaction was carried out for 10 min at 30° C., the reaction was stopped with the addition of sample buffer and after heating the sample was resolved on 10% SDS-PAGE. The aouthoradiography images were scanned and quantified using QuickScan software. The IC₅₀ values were determined using GraFit software.

### Peptide Synthesis

Peptides were assembled using standard solid-phase chemistry based on the Fmoc protecting group was employed³⁸. Peptides were side-chain deprotected and cleaved from the synthesis support using an acidolysis method as described³⁹. All peptides were purified by preparative reversed-phase HPLC, isolated by lyophilization, and analysed by analytical HPLC and mass spectrometry (Dynamo DE MALDI-TOF spectrometer, ThermoBioAnalysis).

### X-Ray Crystallography

PCNA monoclinic form: Crystals of PCNA were grown by the hanging drop vapour diffusion method. A 2-μL solution of PCNA (8˜10 mg/mL) in a buffer consisting of 25 mM Tris and 2 mM DTT was added to 2 μL well solution comprising 20% PEG-3,350 and 0.2 M magnesium acetate. Crystals grew after 7-10 days at 18° C. A crystal of about 0.05 mm in length was collected in a 0.05-0.1 mm cryo-loop (Hampton Research), dipped briefly in immersion oil (Type B, Cargille) and frozen by plunging into liquid nitrogen. The frozen crystal was then transferred to a magnetic goniometer head in a stream of liquid nitrogen at 100 K (Cryostream, Oxford Cryosystems). Diffraction data were collected on a CCD detector using the synchrotron source in Daresbusy station 9.6 (Table 1). X-ray data were processed by DENZO⁴⁰. Molecular replacement was carried out using MOLREP⁴¹ based on the trimeric molecule of the published structure of PCNA complexed with a 22mer p21 peptide (PDB #1AXC) and was calculated within the resolution range of 35-3.1 Å. The rotation function gave three equivalent peaks with R_{f}/σ=10. The next highest peak height was 5.5. The translation function gave an unambiguous peak of T_{f}/σ=26 (next best solution 10), R factor 0.54 (next best solution 0.60) and correlation coefficient of 0.57 (next best solution 0.43). Following the translation function, rigid-body refinement of the optimum molecular replacement solution was performed by the program RIGID in CNS⁴² using data to 3.5 Å. 5% of the data was flagged as R_{free} and omitted from refinement for cross-validation. After 40 cycles of rigid-body refinement, the R-factor reduced from 45.2% to 36.6% (R_{free} from 48.7% to 39.5%).

Following rigid-body refinement, the model was subjected to the stimulated annealing, positional and B-factor refinements by program REFINE in CNS using all data. R-factor reduced from 39.1% to 25.3% (R_{free} from 41.1% to 30.8%) after refinement. ARP/wAR⁴³ was used for initial density interpretation and the addition of water molecules. Subsequent rounds of manual remodelling with the program Quanta (Accelrys, San Diego, USA) and restrained refinement using the program REFMAC⁴⁴ gave an R value of 18.7 and an R_{free} of 27.9.

PCNA trigonal form: The crystal of the trigonal form of PCNA was grown similarly. A 1 μL solution of PCNA (6 mg/mL in 25 mM Tris-HCl, pH 7.5, 1 mM EDTA, 0.01% Nonidet P40, 10% glycerol, 2 mM benzamidine, 1 mM PMSF, 1 mM DTT, and 25 mM NaCl) was added to 1 μL well solution containing 30-40% monomethylated PEG-2,000, 0.1 M sodium acetate buffer (pH 4.6) and 0.2 M ammonium sulfate. Crystals formed after 3-5 days growth at 18° C. A crystal of 0.5 mm in length was dipped into immersion oil and frozen in liquid nitrogen. Diffraction data were collected on a CCD detector using the synchrotron source at Daresbury station 14.1. The data were processed using DENZO⁴⁰. A similar protocol to that used for the monoclinic form was applied for the structure determination; statistics are shown in Table 1.

PCNA-CM peptide complex: Crystals of this complex were grown by the hanging drop vapour diffusion method. A 6-μL solution of PCNA (6-8 mg/mL) and 0.4 mM CM in a buffer consisting of 5 mM HEPES (pH 7.5), 5 mM NaCl and 1.2% v/v DMSO was added to 2 μL well solution comprising 2.7 M ammonium sulfate, HEPES (pH 8.0). Crystals grew after 3˜4 days at 18° C. A crystal of about 0.2 mm in length was collected in a 0.1-0.2 mm cryo-loop (Hampton Research), dipped briefly in 2.7 M ammonium sulphate, HEPES (pH 8.0), 26% glycerol, and was frozen by plunging into liquid nitrogen. The frozen crystal was then transferred to a magnetic goniometer head in a stream of liquid nitrogen at 100 K (Cryostream, Oxford Cryosystems). Diffraction data were collected on MAR345 image plate using station BW7B at DESY, Hamburg. The data were processed using MOSFLM⁴⁵ (Table 1). Molecular replacement was carried out using AMoRe⁴⁶. The trimer molecule of the published structure of hPCNA complexed with the 22mer p21 peptide Waf1 (PDB 1AXC) was used as a starting model. The molecular replacement calculations were performed in the resolution range of 10 Å-3.5 Å. A clear solution was found for two trimers in the asymmetric unit with an R-factor of 53% and a correlation coefficient of 0.30. The rotation function gave three equivalent peaks with Rf/σ around 12 and three more with Rf/σ around 10. The next highest peak was 6. The three unique rotation solutions were used in translation search. The translation function gave an unambiguous peak of correlation intensity=35.1% (next best solution 26.6), R factor 47% (next best solution 50%). The first solution was fixed and translation search performed again. With the second trimer found, correlation intensity increased to 60% and the R factor dropped to 39%. Ten cycles of rigid body refinement using the program REFMAC gave R factor and R_{free} values of 29% and 33%, respectively. ARP/wARP⁴³ was used for initial density interpretation and the addition of water molecules. Subsequent rounds of manual remodelling with the program Quanta (Accelrys, San Diego, USA) and restrained refinement using the program REFMAC⁴⁴ gave an R value of 17.9% and an R_{free} of 25.8%.

Various modifications and variations of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in the relevant fields are intended to be covered by the present invention.

### REFERENCES

1. Prelich, G. et al. Functional identity of proliferating cell nuclear antigen and a DNA polymerase-delta auxiliary protein. Nature 326, 517-20 (1987).

2. Krishna, T. S. R., Kong, X.-P., Gary, S., Burgers, P. M. & Kuriyan, J. Crystal structure of the eukaryotic DNA polymerase processivity factor PCNA. Cell 79, 1233-1243 (1994).

3. Warbrick, E. The puzzle of PCNA's many partners. Bioessays 22, 997-1006 (2000).

4. Gartel, A. L. & Tyner, A. L. The role of the cyclin-dependent kinase inhibitor p21 in apoptosis. Mol. Cancer Ther. 1, 639-49 (2002).

5. Waga, S., Hannon, G. J., Beach, D. & Stillman, B. The p21 inhibitor of cyclin-dependent kinases controls DNA replication by interaction with PCNA. Nature 369, 574-8 (1994).

6. Flores-Rozas, H. et al. Cdk-interacting protein 1 directly binds with proliferating cell nuclear antigen and inhibits DNA replication catalyzed by the DNA polymerase delta holoenzyme. Proc. Natl. Acad. Sci. USA 91, 8655-9 (1994).

7. Shivji, M. K. K., Ferrari, E., Ball, K., Hübscher, U. & Wood, R. D. Resistance of human nucleotide excision repair synthesis in vitro to p21^{Cdn21}. Oncogene 17, 2827-2838 (1998).

8. Savio, M., Stivala, L. A., Scovassi, A. I., Bianchi, L. & Prosperi, E. p21waf1/cip1 protein associates with the detergent-insoluble form of PCNA concomitantly with disassembly of PCNA at nucleotide excision repair sites. Oncogene 13, 1591-8 (1996).

9. Chen, J. et al. A 39 amino acid fragment of the cell cycle regulator p21 is sufficient to bind PCNA and partially inhibit DNA replication in vivo. Nucleic Acids Res. 24, 1727-1733 (1996).

10. Fotedar, R. et al. p21 contains independent binding sites for cyclin and cdk2: both sites are required to inhibit cdk2 kinase activity. Oncogene 12, 2155-64 (1996).

11. Gulbis, J. M., Kelman, Z., Hurwitz, J., O'Donnell, M. & Kuriyan, J. Structure of the C-terminal region of p21(WAF1/CIP1) complexed with human PCNA. Cell 87, 297-306 (1996).

12. Goubin, F. & Ducommun, B. Identification of binding domains on the p21Cip1 cyclin-dependent kinase inhibitor. Oncogene 10, 2281-7 (1995).

13. Cox, L. S. Who binds wins: competition for PCNA rings out cell-cycle changes. Trends Cell Biol. 7, 493-498 (1997).

14. Xiong, Y., Zhang, H. & Beach, D. D type cyclins associate with multiple protein kinases and the DNA replication and repair factor PCNA. Cell 71, 504-14 (1992).

15. Xiong, Y., Zhang, H. & Beach, D. Subunit rearrangement of the cyclin-dependent kinases is associated with cellular transformation. Genes Dev. 7, 1572-83 (1993).

16. Tsurimoto, T. PCNA, a multifunctional ring on DNA. Biochim. Biophys. Acta 26, 1-2 (1998).

17. Paunesku, T. et al. Proliferating cell nuclear antigen (PCNA): ringmaster of the genome. Int. J. Radiat. Biol. 77, 1007-21 (2001).

18. Sakakura, C. et al. Inhibition of gastric cancer cell proliferation by antisense oligonucleotides targeting the messenger RNA encoding proliferating cell nuclear antigen. Br. J. Cancer 70, 1060-6 (1994).

19. Maeshima, Y., Kashihara, N., Sugiyama, H., Makino, H. & Ota, Z. Antisense oligonucleotides to proliferating cell nuclear antigen and Ki-67 inhibit human mesangial cell proliferation. J. Amer. Soc. Nephrol. 7, 2219-29 (1996).

20. Speir, E. & Epstein, S. E. Inhibition of smooth muscle cell proliferation by an antisense oligodeoxynucleotide targeting the messenger RNA encoding proliferating cell nuclear antigen. Circulation 86, 538-47 (1992).

21. Morita, Y. et al. Inhibition of rheumatoid synovial fibroblast proliferation by antisense oligonucleotides targeting proliferating cell nuclear antigen messenger RNA. Arthritis And Rheumatism 40, 1292-7 (1997).

22. Fischer, P. M., Krausz, E. & Lane, D. P. Cellular delivery of impermeable effector molecules in the form of conjugates with peptides capable of mediating membrane translocation. Bioconjugate Chem. 12, 825-841 (2001).

23. Ball, K. L., Lain, S., Fahraeus, R., Smythe, C. & Lane, D. P. Cell-cycle arrest and inhibition of Cdk4 activity by small peptides based on the carboxy-terminal domain of p21WAF1. Curr. Biol. 7, 71-80 (1996).

24. Fåhraeus, R., Paramio, J. M., Ball, K. L., Lain, S. & Lane, D. P. Inhibition of pRb phosphorylation and cell-cycle progression by a 20-residue peptide derived from p16CDKN2/INK4A. Curr. Biol. 6, 84-91 (1996).

25. Warbrick, E., Lane, D. P., Glover, D. M. & Cox, L. S. A small peptide inhibitor of DNA replication defines the site of interaction between the cyclin-dependent kinase inhibitor p21WAF1 and proliferating cell nuclear antigen. Curr. Biol. 5, 275-282 (1995).

26. Zheleva, D. I. et al. Highly potent p₂₁^{WAF1} derived peptide inhibitors of CDK-mediated pRb phosphorylation: delineation and structural insight into their interactions with cyclin A. J. Peptide Res. 60, 257-270 (2002).

27. Kontopidis, G. et al. Insights into cyclin groove recognition: complex crystal structures and inhibitor design through ligand exchange. Structure 11, 1537-1546 (2003).

28. Zheleva, D. I., Zhelev, N. Z., Fischer, P. M., Duff, S. V. & Lane, D. P. A quantitative study of the in vitro binding of the C-terminal domain of p21 to PCNA—affinity, stoichiometry, and thermodynamics. Biochemistry 39, 7388-7397 (2000).

29. Vairapandi, M., Azam, N., Balliet, A. G., Hoffman, B. & Liebermann, D. A. Characterization of MyD118, Gadd45, and proliferating cell nuclear antigen (PCNA) interacting domains. PCNA impedes MyD118 AND Gadd45-mediated negative growth control. J. Biol. Chem. 275, 16810-9 (2000).

30. Clark, A. B., Valle, F., Drotschmann, K., Gary, R. K. & Kunkel, T. A. Functional interaction of proliferating cell nuclear antigen with MSH2-MSH6 and MSH2-MSH3 complexes. J. Biol. Chem. 275, 36498-501 (2000).

31. Ling, X. et al. Proliferating cell nuclear antigen as the cell cycle sensor for an HLA-derived peptide blocking T cell proliferation. J. Immunol. 164, 6188-92 (2000).

32. Chapados, B. R. et al. Structural basis for FEN-1 substrate specificity and PCNA-mediated activation in DNA replication and repair. Cell 116, 39-50 (2004).

33. Matsuoka, S., Yamaguchi, M. & Matsukage, A. D-type cyclin-binding regions of proliferating cell nuclear antigen. J Biol. Chem. 269, 11030-6 (1994).

34. McInnes, C., Andrews, M. J. I., Zheleva, D. I., Lane, D. P. & Fischer, P. M. Peptidomimetic design of CDK inhibitors targeting the recruitment site of the cyclin subunit. Curr. Med. Chem. Anti-Cancer Agents 3, 57-69 (2003).

35. Ando, T. et al. Involvement of the interaction between p21 and proliferating cell nuclear antigen for the maintenance of G2/M arrest after DNA damage. J. Biol. Chem. 276, 42971-7 (2001).

36. Koundrioukoff, S. et al. A direct interaction between proliferating cell nuclear antigen (PCNA) and Cdk2 targets PCNA-interacting proteins for phosphorylation. J. Biol. Chem. 275, 22882-7 (2000).

37. Scott, M. T., Morrice, N. & Ball, K. L. Reversible phosphorylation at the C-terminal regulatory domain of p^{21Waf1/Cip1} modulates proliferating cell nuclear antigen binding. J. Biol. Chem. 275, 11529-11537 (2000).

38. Chan, W. C. & White, P. D. (eds.). Fmoc Solid Phase Peptide Synthesis: A Practical Approach, (Oxford University Press, Oxford, 2000).

39. King, D. S., Fields, C. G. & Fields, G. B. A cleavage method which minimizes side reactions following Fmoc solid phase peptide synthesis. Int. J. Peptide Protein Res. 36, 255-266 (1990).

40. Otwinowski, Z. & Minor, W. Processing of x-ray diffraction data collected in oscillation mode. Methods Enzymol. 276, 307-326 (1997).

41. Vagin, A. & Teplyakov, A. MOLREP: an automated program for molecular replacement. J. Appl. Crystallogr. 30, 1022-1025 (1997).

42. Brunger, A. T. et al. Crystallography & NMR System: a new software suite for macromolecular structure determination. Acta Crystallogr. D54, 905-921 (1998).

43. Lamzin, V. S. & Wilson, K. S. Automated refinement for protein crystallography. Methods Enzymol. 277, 269-305 (1997).

44. Murshudov, G. N., Vagin, A. A. & Dodson, E. J. Refinement of macromolecular structures by the maximum-likelihood method. Acta Crystallogr. D53, 240-255 (1997).

45. Leslie, A. G. W. Recent changes to the MOSFLM package for processing film and image plate data. Joint CCP4+ESF-EAMCB Newsletter on Protein Crystallography 26(1992).

46. Navaza, J. AMoRe: an automated package for molecular replacement. Acta Crystallogr. A50, 157-63 (1994).

TABLE 1
X-ray data and refinement statistics

| | | | | hPCNA-CM |
|---|---|---|---|---|
| | | hPCNA | hPCNA | peptide |
| | | monoclinic | trigonal | complex |
| Space Group | | C121 | P3 | |
| Unit Cell | a (Å) | 136.6 | 82.89 | 119.1 |
| | b (Å) | 83.26 | 82.89 | 119.1 |
| | c (Å) | 71.63 | 70.86 | 305.82 |
| | β (°) | 117.49 | 120 | 120 |
| Z (monomers per unit cell) | | 12 | 6 | 6 |
| Resolution Range (Å) | | 35-2.3 | 35-3.1 | 30-2.78 |
| Reflections | No. measured | 220,606 | 9,268 | 241,549 |
| | Unique | 31,758 | 6,026 | 64,087 |
| Completeness¹ (%) | | 99.4 | 98.0 (98.4) | 99.6 (99.7) |
| R_{merge} (outer shell) | | 5.8 (45.7) | 8.8 (28) | 14 (67) |
| I/σ (I) (outer shell) | | 15.5 (2.7) | 12.9 (4.8) | 2.8 (1.0) |
| Multiplicity | | 6.95 | 1.5 | 3.8 |
| R-factor (%) | | 18.7 | 18.3 | 17.9 |
| R_{free} (%) | | 27.9 | 27.3 | 25.8 |
| % data used to calculate R_{free} | | 4.1 | 4.8 | 3.1 |
| Number of atoms | Protein | 5,889 | 3,926 | 11,919 |
| | Peptide | — | — | 810 |
| | Water | 148 | 58 | 322 |
| RMS deviation | Bonds (Å) | 0.014 | 0.012 | 0.015 |
| | Angles (°) | 1.68 | 1.47 | 2.04 |
| Average B-factors (Å²) | Protein | 51.2 | 50.4 | 57.4 |
| | Peptide | — | | 74.5 |
| | Water | 51.0 | 46.8 | 53.4 |

¹The numbers in parenthesis are statistics for the highest resolution shell.
R_{merge} = Σₕ |I − <I> |/Σₕ| I|, where <I> is the mean intensity of all observations of reflection h = hkl.
σ (I) is the SD of the measured intensity.

TABLE 2
Comparison of binding energies of p21^{WAF1} (1AXC)
and CM peptides with PCNA

| | |
|---|---|
| Core | Affinity score |
| residues* | (kcal/mol) |

| p21 | CM | p21(143-160) | CM(1-16) |
|---|---|---|---|
| Q144 | Q5 | −12.2 | −10.3 |
| T145 | K6 | −7.79 | −77.1 |
| S146 | K7 | −4.94 | −84.8 |
| M147 | I8 | −6.71 | −3.55 |
| T148 | T9 | −2.45 | −2.23 |
| D149 | D10 | 76.8 | 76.0 |
| F150 | Y11 | −7.95 | −9.54 |
| Y151 | F12 | −7.19 | −11.7 |
| H152 | H13 | −1.97 | −4.17 |

*core residues: Residues 5-13 in CM and 144-153 in p21 peptides.

TABLE 3
Coordinates of the complex of human PCNA of space group C121 with unit
dimensions: a = 136.6 Å, b = 83.26 Å, c = 71.63 Å.

| | | | |
|---|---|---|---|
| REMARK | 3 | REFINEMENT. | |
| REMARK | 3 | PROGRAM | : REFMAC 5.1.24 |
| REMARK | 3 | AUTHORS | : MURSHUDOV, VAGIN, DODSON |
| REMARK | 3 | | |

| REMARK | 3 | REFINEMENT TARGET: MAXIMUM LIKELIHOOD | | |
|---|---|---|---|---|
| REMARK | 3 | | | |
| REMARK | 3 | DATA USED IN REFINEMENT. | | |
| REMARK | 3 | RESOLUTION RANGE HIGH | (ANGSTROMS) | : 2.30 |
| REMARK | 3 | RESOLUTION RANGE LOW | (ANGSTROMS) | : 14.00 |
| REMARK | 3 | DATA CUTOFF | (SIGMA(F)) | : NONE |
| REMARK | 3 | COMPLETENESS FOR RANGE | (%) | : 99.41 |
| REMARK | 3 | NUMBER OF REFLECTIONS | | : 30139 |
| REMARK | 3 | | | |

| REMARK | 3 | FIT TO DATA USED IN REFINEMENT. | | |
|---|---|---|---|---|
| REMARK | 3 | CROSS-VALIDATION METHOD | | : THROUGHOUT |
| REMARK | 3 | FREE R VALUE TEST SET SELECTION | | : RANDOM |
| REMARK | 3 | R VALUE | (WORKING + TEST SET) | : 0.19123 |
| REMARK | 3 | R VALUE | (WORKING SET) | : 0.18751 |

| REMARK | 3 | FREE R VALUE | | : 0.27936 |
|---|---|---|---|---|
| REMARK | 3 | FREE R VALUE TEST SET SIZE | (%) | : 4.1 |

| | | | |
|---|---|---|---|
| REMARK | 3 | FREE R VALUE TEST SET COUNT | : 1274 |
| REMARK | 3 | | |

| REMARK | 3 | FIT IN THE HIGHEST RESOLUTION BIN. | | |
|---|---|---|---|---|
| REMARK | 3 | TOTAL NUMBER OF BINS USED | : | 20 |
| REMARK | 3 | BIN RESOLUTION RANGE HIGH | : | 2.300 |
| REMARK | 3 | BIN RESOLUTION RANGE LOW | : | 2.358 |

| | | | | |
|---|---|---|---|---|
| REMARK | 3 | REFLECTION IN BIN | (WORKING SET) : | 2143 |
| REMARK | 3 | BIN R VALUE | (WORKING SET) : | 0.250 |

| | | | | |
|---|---|---|---|---|
| REMARK | 3 | BIN FREE R VALUE SET COUNT | : | 83 |
| REMARK | 3 | BIN FREE R VALUE | : | 0.381 |
| REMARK | 3 | | | |

| REMARK | 3 | NUMBER OF NON-HYDROGEN ATOMS USED IN REFINEMENT. | | |
|---|---|---|---|---|
| REMARK | 3 | ALL ATOMS | : | 6037 |
| REMARK | 3 | | | |
| REMARK | 3 | B VALUES. | | |

| | | | | |
|---|---|---|---|---|
| REMARK | 3 | FROM WILSON PLOT | (A**2) : | NULL |
| REMARK | 3 | MEAN B VALUE | (OVERALL, A**2) : | 47.421 |

| REMARK | 3 | OVERALL ANISOTROPIC B VALUE. | |
|---|---|---|---|
| REMARK | 3 | B11 (A**2) : | 0.48 |
| REMARK | 3 | B22 (A**2) : | −1.07 |
| REMARK | 3 | B33 (A**2) : | −0.85 |
| REMARK | 3 | B12 (A**2) : | 0.00 |
| REMARK | 3 | B13 (A**2) : | −1.56 |
| REMARK | 3 | B23 (A**2) : | 0.00 |
| REMARK | 3 | | |

| REMARK | 3 | ESTIMATED OVERALL COORDINATE ERROR. | | |
|---|---|---|---|---|
| REMARK | 3 | ESU BASED ON R VALUE | (A): | 0.440 |
| REMARK | 3 | ESU BASED ON FREE R VALUE | (A): | 0.292 |
| REMARK | 3 | ESU BASED ON MAXIMUM LIKELIHOOD | (A): | 0.210 |
| REMARK | 3 | ESU FOR B VALUES BASED ON MAXIMUM LIKELIHOOD | (A**2): | 8.520 |
| REMARK | 3 | | | |
| REMARK | 3 | CORRELATION COEFFICIENTS. | | |

| | | | |
|---|---|---|---|
| REMARK | 3 | CORRELATION COEFFICIENT FO-FC | : 0.959 |
| REMARK | 3 | CORRELATION COEFFICIENT FO-FC FREE | : 0.903 |
| REMARK | 3 | | |

| REMARK | 3 | RMS DEVIATIONS FROM IDEAL VALUES | | COUNT | RMS | WEIGHT |
|---|---|---|---|---|---|---|
| REMARK | 3 | BOND LENGTHS REFINED ATOMS | (A): | 5967; | 0.014; | 0.022 |
| REMARK | 3 | BOND LENGTHS OTHERS | (A): | 5490; | 0.002; | 0.020 |
| REMARK | 3 | BOND ANGLES REFINED ATOMS | (DEGREES): | 8061; | 1.680; | 1.977 |
| REMARK | 3 | BOND ANGLES OTHERS | (DEGREES): | 12843; | 0.808; | 3.000 |
| REMARK | 3 | TORSION ANGLES, PERIOD 1 | (DEGREES): | 762; | 9.433; | 5.000 |
| REMARK | 3 | CHIRAL-CENTER RESTRAINTS | (A**3): | 954; | 0.119; | 0.200 |

| | | | | | | |
|---|---|---|---|---|---|---|
| REMARK | 3 | GENERAL PLANES REFINED ATOMS | (A): | 6579; | 0.010; | 0.020 |
| REMARK | 3 | GENERAL PLANES OTHERS | (A): | 1074; | 0.003; | 0.020 |
| REMARK | 3 | NON-BONDED CONTACTS REFINED ATOMS | (A): | 1137; | 0.254; | 0.300 |
| REMARK | 3 | NON-BONDED CONTACTS OTHERS | (A): | 6315; | 0.259; | 0.300 |
| REMARK | 3 | NON-BONDED TORSION OTHERS | (A): | 4149; | 0.099; | 0.500 |
| REMARK | 3 | H-BOND (X . . . Y) REFINED ATOMS | (A): | 257; | 0.250; | 0.500 |
| REMARK | 3 | SYMMETRY VDW REFINED ATOMS | (A): | 59; | 0.286; | 0.300 |
| REMARK | 3 | SYMMETRY VDW OTHERS | (A): | 122; | 0.305; | 0.300 |
| REMARK | 3 | SYMMETRY H-BOND REFINED ATOMS | (A): | 18; | 0.327; | 0.500 |
| REMARK | 3 | | | | | |

| REMARK | 3 | ISOTROPIC THERMAL FACTOR RESTRAINTS. | | COUNT | RMS | WEIGHT |
|---|---|---|---|---|---|---|
| REMARK | 3 | MAIN-CHAIN BOND REFINED ATOMS | (A**2): | 3804; | 5.143; | 1.500 |
| REMARK | 3 | MAIN-CHAIN ANGLE REFINED ATOMS | (A**2): | 6141; | 7.507; | 2.000 |
| REMARK | 3 | SIDE-CHAIN BOND REFINED ATOMS | (A**2): | 2163; | 10.489; | 3.000 |
| REMARK | 3 | SIDE-CHAIN ANGLE REFINED ATOMS | (A**2): | 1920; | 15.143; | 4.500 |
| REMARK | 3 | | | | | |

| REMARK | 3 | NCS RESTRAINTS STATISTICS | |
|---|---|---|---|
| REMARK | 3 | NUMBER OF NCS GROUPS: NULL | |
| REMARK | 3 | | |
| REMARK | 3 | | |
| REMARK | 3 | TLS DETAILS | |
| REMARK | 3 | NUMBER OF TLS GROUPS: NULL | |
| REMARK | 3 | | |
| REMARK | 3 | | |
| REMARK | 3 | BULK SOLVENT MODELLING. | |
| REMARK | 3 | METHOD USED: BABINET MODEL WITH MASK | |
| REMARK | 3 | PARAMETERS FOR MASK CALCULATION | |
| REMARK | 3 | VDW PROBE RADIUS | : 1.40 |
| REMARK | 3 | ION PROBE RADIUS | : 0.80 |
| REMARK | 3 | SHRINKAGE RADIUS | : 0.80 |
| REMARK | 3 | | |

| | | |
|---|---|---|
| REMARK | 3 | OTHER REFINEMENT REMARKS: |
| REMARK | 3 | HYDROGENS HAVE BEEN ADDED IN THE RIDING POSITIONS |
| REMARK | 3 | |

| | | | | | | | |
|---|---|---|---|---|---|---|---|
| CRYST1 | 136.653 | 83.265 | 71.634 | 90.00 | 117.49 | 90.00 | C 1 2 1 |

| | | | | |
|---|---|---|---|---|
| SCALE1 | 0.007318 | 0.000000 | 0.003808 | 0.00000 |
| SCALE2 | 0.000000 | 0.012010 | 0.000000 | 0.00000 |
| SCALE3 | 0.000000 | 0.000000 | 0.015737 | 0.00000 |

| | | | | | | | | | | | |
|---|---|---|---|---|---|---|---|---|---|---|---|
| ATOM | 1 | N | MET | A | 1 | 35.718 | −7.747 | 39.704 | 1.00 | 54.17 | N |
| ATOM | 3 | CA | MET | A | 1 | 34.511 | −8.129 | 38.940 | 1.00 | 61.78 | C |
| ATOM | 5 | CB | MET | A | 1 | 33.666 | −9.100 | 39.775 | 1.00 | 62.68 | C |
| ATOM | 8 | CG | MET | A | 1 | 32.603 | −9.833 | 38.987 | 1.00 | 69.62 | C |
| ATOM | 11 | SD | MET | A | 1 | 30.919 | −9.327 | 39.414 | 1.00 | 85.22 | S |
| ATOM | 12 | CE | MET | A | 1 | 29.953 | −10.512 | 38.448 | 1.00 | 83.13 | C |
| ATOM | 16 | C | MET | A | 1 | 33.661 | −6.915 | 38.524 | 1.00 | 59.30 | C |
| ATOM | 17 | O | MET | A | 1 | 33.558 | −5.897 | 39.236 | 1.00 | 59.87 | O |
| ATOM | 20 | N | PHE | A | 2 | 33.007 | −7.070 | 37.379 | 1.00 | 55.51 | N |
| ATOM | 22 | CA | PHE | A | 2 | 32.045 | −6.095 | 36.877 | 1.00 | 48.63 | C |
| ATOM | 24 | CB | PHE | A | 2 | 32.679 | −5.273 | 35.767 | 1.00 | 47.68 | C |
| ATOM | 27 | CG | PHE | A | 2 | 31.702 | −4.407 | 35.029 | 1.00 | 61.53 | C |
| ATOM | 28 | CD1 | PHE | A | 2 | 30.896 | −3.524 | 35.720 | 1.00 | 51.03 | C |
| ATOM | 30 | CE1 | PHE | A | 2 | 29.997 | −2.741 | 35.043 | 1.00 | 61.36 | C |
| ATOM | 32 | CZ | PHE | A | 2 | 29.872 | −2.853 | 33.688 | 1.00 | 48.45 | C |
| ATOM | 34 | CE2 | PHE | A | 2 | 30.656 | −3.742 | 32.996 | 1.00 | 51.79 | C |
| ATOM | 36 | CD2 | PHE | A | 2 | 31.535 | −4.534 | 33.658 | 1.00 | 37.09 | C |
| ATOM | 38 | C | PHE | A | 2 | 30.828 | −6.821 | 36.328 | 1.00 | 45.37 | C |
| ATOM | 39 | O | PHE | A | 2 | 30.949 | −7.825 | 35.645 | 1.00 | 49.07 | O |
| ATOM | 40 | N | GLU | A | 3 | 29.648 | −6.303 | 36.619 | 1.00 | 43.97 | N |
| ATOM | 42 | CA | GLU | A | 3 | 28.450 | −6.798 | 35.991 | 1.00 | 45.06 | C |
| ATOM | 44 | CB | GLU | A | 3 | 27.983 | −8.045 | 36.724 | 1.00 | 47.08 | C |
| ATOM | 47 | CG | GLU | A | 3 | 26.528 | −8.377 | 36.455 | 1.00 | 61.88 | C |
| ATOM | 50 | CD | GLU | A | 3 | 26.177 | −9.754 | 36.977 | 1.00 | 63.51 | C |
| ATOM | 51 | OE1 | GLU | A | 3 | 24.984 | −10.060 | 37.185 | 1.00 | 88.81 | O |
| ATOM | 52 | OE2 | GLU | A | 3 | 27.120 | −10.524 | 37.196 | 1.00 | 58.19 | O |
| ATOM | 53 | C | GLU | A | 3 | 27.358 | −5.744 | 36.044 | 1.00 | 43.06 | C |
| ATOM | 54 | O | GLU | A | 3 | 26.986 | −5.299 | 37.122 | 1.00 | 36.39 | O |
| ATOM | 55 | N | ALA | A | 4 | 26.805 | −5.406 | 34.881 | 1.00 | 45.88 | N |
| ATOM | 57 | CA | ALA | A | 4 | 25.715 | −4.444 | 34.784 | 1.00 | 38.79 | C |
| ATOM | 59 | CB | ALA | A | 4 | 26.201 | −3.121 | 34.220 | 1.00 | 39.58 | C |
| ATOM | 63 | C | ALA | A | 4 | 24.620 | −5.044 | 33.905 | 1.00 | 43.01 | C |
| ATOM | 64 | O | ALA | A | 4 | 24.867 | −5.518 | 32.785 | 1.00 | 41.26 | O |
| ATOM | 65 | N | ARG | A | 5 | 23.404 | −5.035 | 34.438 | 1.00 | 35.18 | N |
| ATOM | 67 | CA | ARG | A | 5 | 22.276 | −5.613 | 33.747 | 1.00 | 30.80 | C |
| ATOM | 69 | CB | ARG | A | 5 | 21.580 | −6.630 | 34.649 | 1.00 | 34.25 | C |
| ATOM | 72 | CG | ARG | A | 5 | 20.267 | −7.192 | 34.188 | 1.00 | 38.70 | C |
| ATOM | 75 | CD | ARG | A | 5 | 19.648 | −8.140 | 35.237 | 1.00 | 49.06 | C |
| ATOM | 78 | NE | ARG | A | 5 | 18.195 | −8.008 | 35.326 | 1.00 | 61.82 | N |
| ATOM | 80 | CZ | ARG | A | 5 | 17.323 | −8.785 | 34.685 | 1.00 | 76.56 | C |
| ATOM | 81 | NH1 | ARG | A | 5 | 17.751 | −9.786 | 33.912 | 1.00 | 52.60 | N |
| ATOM | 84 | NH2 | ARG | A | 5 | 16.014 | −8.568 | 34.830 | 1.00 | 71.44 | N |
| ATOM | 87 | C | ARG | A | 5 | 21.380 | −4.481 | 33.317 | 1.00 | 32.95 | C |
| ATOM | 88 | O | ARG | A | 5 | 21.021 | −3.586 | 34.076 | 1.00 | 36.76 | O |
| ATOM | 89 | N | LEU | A | 6 | 21.033 | −4.550 | 32.048 | 1.00 | 34.06 | N |
| ATOM | 91 | CA | LEU | A | 6 | 20.211 | −3.572 | 31.397 | 1.00 | 28.83 | C |
| ATOM | 93 | CB | LEU | A | 6 | 21.078 | −2.830 | 30.394 | 1.00 | 26.41 | C |
| ATOM | 96 | CG | LEU | A | 6 | 20.446 | −1.673 | 29.634 | 1.00 | 55.50 | C |
| ATOM | 98 | CD1 | LEU | A | 6 | 19.721 | −0.696 | 30.576 | 1.00 | 60.97 | C |
| ATOM | 102 | CD2 | LEU | A | 6 | 21.527 | −0.968 | 28.835 | 1.00 | 51.31 | C |
| ATOM | 106 | C | LEU | A | 6 | 19.035 | −4.294 | 30.706 | 1.00 | 41.55 | C |
| ATOM | 107 | O | LEU | A | 6 | 19.212 | −4.971 | 29.683 | 1.00 | 39.71 | O |
| ATOM | 108 | N | VAL | A | 7 | 17.837 | −4.171 | 31.280 | 1.00 | 37.73 | N |
| ATOM | 110 | CA | VAL | A | 7 | 16.625 | −4.724 | 30.664 | 1.00 | 38.59 | C |
| ATOM | 112 | CB | VAL | A | 7 | 15.423 | −4.753 | 31.679 | 1.00 | 36.55 | C |
| ATOM | 114 | CG1 | VAL | A | 7 | 14.042 | −4.822 | 30.983 | 1.00 | 47.73 | C |
| ATOM | 118 | CG2 | VAL | A | 7 | 15.546 | −5.986 | 32.550 | 1.00 | 35.66 | C |
| ATOM | 122 | C | VAL | A | 7 | 16.238 | −4.088 | 29.323 | 1.00 | 37.34 | C |
| ATOM | 123 | O | VAL | A | 7 | 15.871 | −4.813 | 28.395 | 1.00 | 32.80 | O |
| ATOM | 124 | N | GLN | A | 8 | 16.353 | −2.760 | 29.198 | 1.00 | 43.19 | N |
| ATOM | 126 | CA | GLN | A | 8 | 16.082 | −2.081 | 27.926 | 1.00 | 39.20 | C |
| ATOM | 128 | CB | GLN | A | 8 | 15.641 | −0.631 | 28.177 | 1.00 | 44.68 | C |
| ATOM | 131 | CG | GLN | A | 8 | 14.137 | −0.390 | 28.170 | 1.00 | 52.20 | C |
| ATOM | 134 | CD | GLN | A | 8 | 13.781 | 1.076 | 27.978 | 1.00 | 58.41 | C |
| ATOM | 135 | OE1 | GLN | A | 8 | 14.480 | 1.967 | 28.465 | 1.00 | 70.76 | O |
| ATOM | 136 | NE2 | GLN | A | 8 | 12.709 | 1.327 | 27.235 | 1.00 | 59.52 | N |
| ATOM | 139 | C | GLN | A | 8 | 17.373 | −2.033 | 27.105 | 1.00 | 41.27 | C |
| ATOM | 140 | O | GLN | A | 8 | 17.940 | −0.962 | 26.910 | 1.00 | 41.84 | O |
| ATOM | 141 | N | GLY | A | 9 | 17.835 | −3.176 | 26.618 | 1.00 | 42.65 | N |
| ATOM | 143 | CA | GLY | A | 9 | 19.066 | −3.221 | 25.862 | 1.00 | 45.16 | C |
| ATOM | 146 | C | GLY | A | 9 | 19.087 | −2.398 | 24.585 | 1.00 | 40.39 | C |
| ATOM | 147 | O | GLY | A | 9 | 20.149 | −2.260 | 23.981 | 1.00 | 45.96 | O |
| ATOM | 148 | N | SER | A | 10 | 17.940 | −1.865 | 24.166 | 1.00 | 42.48 | N |
| ATOM | 150 | CA | SER | A | 10 | 17.861 | −1.111 | 22.908 | 1.00 | 40.97 | C |
| ATOM | 152 | CB | SER | A | 10 | 16.419 | −0.762 | 22.548 | 1.00 | 38.68 | C |
| ATOM | 155 | OG | SER | A | 10 | 15.820 | 0.012 | 23.574 | 1.00 | 50.33 | O |
| ATOM | 157 | C | SER | A | 10 | 18.694 | 0.157 | 23.009 | 1.00 | 38.61 | C |
| ATOM | 158 | O | SER | A | 10 | 19.333 | 0.573 | 22.033 | 1.00 | 40.96 | O |
| ATOM | 159 | N | ILE | A | 11 | 18.771 | 0.686 | 24.229 | 1.00 | 34.67 | N |
| ATOM | 161 | CA | ILE | A | 11 | 19.563 | 1.879 | 24.512 | 1.00 | 39.05 | C |
| ATOM | 163 | CB | ILE | A | 11 | 19.465 | 2.283 | 25.997 | 1.00 | 41.86 | C |
| ATOM | 165 | CG1 | ILE | A | 11 | 18.108 | 2.942 | 26.305 | 1.00 | 53.48 | C |
| ATOM | 168 | CD1 | ILE | A | 11 | 17.606 | 2.750 | 27.772 | 1.00 | 51.64 | C |
| ATOM | 172 | CG2 | ILE | A | 11 | 20.560 | 3.260 | 26.332 | 1.00 | 40.76 | C |
| ATOM | 176 | C | ILE | A | 11 | 21.022 | 1.648 | 24.164 | 1.00 | 35.86 | C |
| ATOM | 177 | O | ILE | A | 11 | 21.663 | 2.509 | 23.593 | 1.00 | 37.48 | O |
| ATOM | 178 | N | LEU | A | 12 | 21.564 | 0.498 | 24.519 | 1.00 | 31.34 | N |
| ATOM | 180 | CA | LEU | A | 12 | 22.992 | 0.284 | 24.326 | 1.00 | 38.27 | C |
| ATOM | 182 | CB | LEU | A | 12 | 23.485 | −0.834 | 25.248 | 1.00 | 35.72 | C |
| ATOM | 185 | CG | LEU | A | 12 | 25.000 | −1.025 | 25.166 | 1.00 | 49.17 | C |
| ATOM | 187 | CD1 | LEU | A | 12 | 25.771 | 0.217 | 25.573 | 1.00 | 46.34 | C |
| ATOM | 191 | CD2 | LEU | A | 12 | 25.446 | −2.252 | 25.956 | 1.00 | 60.70 | C |
| ATOM | 195 | C | LEU | A | 12 | 23.304 | 0.002 | 22.845 | 1.00 | 32.44 | C |
| ATOM | 196 | O | LEU | A | 12 | 24.353 | 0.373 | 22.330 | 1.00 | 38.14 | O |
| ATOM | 197 | N | LYS | A | 13 | 22.328 | −0.555 | 22.142 | 1.00 | 39.79 | N |
| ATOM | 199 | CA | LYS | A | 13 | 22.416 | −0.686 | 20.698 | 1.00 | 38.56 | C |
| ATOM | 201 | CB | LYS | A | 13 | 21.218 | −1.448 | 20.121 | 1.00 | 34.76 | C |
| ATOM | 204 | CG | LYS | A | 13 | 21.148 | −2.923 | 20.517 | 1.00 | 46.30 | C |
| ATOM | 207 | CD | LYS | A | 13 | 19.872 | −3.618 | 19.974 | 1.00 | 33.67 | C |
| ATOM | 210 | CE | LYS | A | 13 | 19.995 | −3.899 | 18.443 | 1.00 | 36.27 | C |
| ATOM | 213 | NZ | LYS | A | 13 | 20.513 | −5.299 | 18.131 | 1.00 | 36.25 | N |
| ATOM | 217 | C | LYS | A | 13 | 22.498 | 0.670 | 20.024 | 1.00 | 37.76 | C |
| ATOM | 218 | O | LYS | A | 13 | 23.256 | 0.795 | 19.066 | 1.00 | 39.13 | O |
| ATOM | 219 | N | LYS | A | 14 | 21.701 | 1.641 | 20.491 | 1.00 | 36.74 | N |
| ATOM | 221 | CA | LYS | A | 14 | 21.694 | 3.013 | 19.955 | 1.00 | 34.64 | C |
| ATOM | 223 | CB | LYS | A | 14 | 20.516 | 3.832 | 20.520 | 1.00 | 32.46 | C |
| ATOM | 226 | CG | LYS | A | 14 | 19.146 | 3.321 | 20.060 | 1.00 | 40.04 | C |
| ATOM | 229 | CD | LYS | A | 14 | 18.031 | 4.075 | 20.744 | 1.00 | 34.52 | C |
| ATOM | 232 | CE | LYS | A | 14 | 16.695 | 3.762 | 20.097 | 1.00 | 43.89 | C |
| ATOM | 235 | NZ | LYS | A | 14 | 15.586 | 3.939 | 21.088 | 1.00 | 39.49 | N |
| ATOM | 239 | C | LYS | A | 14 | 23.001 | 3.797 | 20.201 | 1.00 | 34.63 | C |
| ATOM | 240 | O | LYS | A | 14 | 23.452 | 4.513 | 19.311 | 1.00 | 35.89 | O |
| ATOM | 241 | N | VAL | A | 15 | 23.592 | 3.671 | 21.393 | 1.00 | 28.38 | N |
| ATOM | 243 | CA | VAL | A | 15 | 24.884 | 4.286 | 21.712 | 1.00 | 29.15 | C |
| ATOM | 245 | CB | VAL | A | 15 | 25.244 | 4.017 | 23.161 | 1.00 | 36.36 | C |
| ATOM | 247 | CG1 | VAL | A | 15 | 26.689 | 4.377 | 23.433 | 1.00 | 32.28 | C |
| ATOM | 251 | CG2 | VAL | A | 15 | 24.318 | 4.758 | 24.096 | 1.00 | 38.17 | C |
| ATOM | 255 | C | VAL | A | 15 | 26.046 | 3.754 | 20.856 | 1.00 | 31.06 | C |
| ATOM | 256 | O | VAL | A | 15 | 26.828 | 4.538 | 20.344 | 1.00 | 40.59 | O |
| ATOM | 257 | N | LEU | A | 16 | 26.205 | 2.435 | 20.744 | 1.00 | 34.21 | N |
| ATOM | 259 | CA | LEU | A | 16 | 27.262 | 1.906 | 19.885 | 1.00 | 41.13 | C |
| ATOM | 261 | CB | LEU | A | 16 | 27.420 | 0.390 | 20.004 | 1.00 | 33.15 | C |
| ATOM | 264 | CG | LEU | A | 16 | 28.433 | −0.084 | 21.051 | 1.00 | 32.68 | C |
| ATOM | 266 | CD1 | LEU | A | 16 | 28.021 | 0.404 | 22.426 | 1.00 | 39.95 | C |
| ATOM | 270 | CD2 | LEU | A | 16 | 29.856 | 0.345 | 20.694 | 1.00 | 47.66 | C |
| ATOM | 274 | C | LEU | A | 16 | 27.077 | 2.281 | 18.402 | 1.00 | 41.17 | C |
| ATOM | 275 | O | LEU | A | 16 | 28.077 | 2.476 | 17.708 | 1.00 | 39.89 | O |
| ATOM | 276 | N | GLU | A | 17 | 25.838 | 2.282 | 17.903 | 1.00 | 40.11 | N |
| ATOM | 278 | CA | GLU | A | 17 | 25.570 | 2.772 | 16.538 | 1.00 | 47.41 | C |
| ATOM | 280 | CB | GLU | A | 17 | 24.108 | 2.614 | 16.153 | 1.00 | 45.53 | C |
| ATOM | 283 | CG | GLU | A | 17 | 23.686 | 1.169 | 15.911 | 1.00 | 69.78 | C |
| ATOM | 286 | CD | GLU | A | 17 | 24.705 | 0.384 | 15.099 | 1.00 | 87.57 | C |
| ATOM | 287 | OE1 | GLU | A | 17 | 25.244 | −0.623 | 15.625 | 1.00 | 102.78 | O |
| ATOM | 288 | OE2 | GLU | A | 17 | 24.984 | 0.788 | 13.944 | 1.00 | 88.25 | O |
| ATOM | 289 | C | GLU | A | 17 | 25.958 | 4.240 | 16.387 | 1.00 | 44.32 | C |
| ATOM | 290 | O | GLU | A | 17 | 26.350 | 4.687 | 15.315 | 1.00 | 52.03 | O |
| ATOM | 291 | N | ALA | A | 18 | 25.876 | 4.973 | 17.487 | 1.00 | 41.83 | N |
| ATOM | 293 | CA | ALA | A | 18 | 26.324 | 6.354 | 17.547 | 1.00 | 43.43 | C |
| ATOM | 295 | CB | ALA | A | 18 | 25.707 | 7.043 | 18.777 | 1.00 | 36.68 | C |
| ATOM | 299 | C | ALA | A | 18 | 27.853 | 6.553 | 17.574 | 1.00 | 45.28 | C |
| ATOM | 300 | O | ALA | A | 18 | 28.320 | 7.692 | 17.434 | 1.00 | 40.53 | O |
| ATOM | 301 | N | LEU | A | 19 | 28.628 | 5.509 | 17.861 | 1.00 | 43.91 | N |
| ATOM | 303 | CA | LEU | A | 19 | 30.046 | 5.710 | 18.163 | 1.00 | 41.74 | C |
| ATOM | 305 | CB | LEU | A | 19 | 30.393 | 5.102 | 19.519 | 1.00 | 41.11 | C |
| ATOM | 308 | CG | LEU | A | 19 | 29.889 | 5.763 | 20.804 | 1.00 | 50.94 | C |
| ATOM | 310 | CD1 | LEU | A | 19 | 30.137 | 4.819 | 21.956 | 1.00 | 52.99 | C |
| ATOM | 314 | CD2 | LEU | A | 19 | 30.578 | 7.097 | 21.071 | 1.00 | 45.43 | C |
| ATOM | 318 | C | LEU | A | 19 | 30.937 | 5.070 | 17.107 | 1.00 | 42.48 | C |
| ATOM | 319 | O | LEU | A | 19 | 32.073 | 5.475 | 16.913 | 1.00 | 45.60 | O |
| ATOM | 320 | N | LYS | A | 20 | 30.420 | 4.034 | 16.461 | 1.00 | 46.46 | N |
| ATOM | 322 | CA | LYS | A | 20 | 31.245 | 3.108 | 15.692 | 1.00 | 45.80 | C |
| ATOM | 324 | CB | LYS | A | 20 | 30.427 | 1.870 | 15.299 | 1.00 | 42.76 | C |
| ATOM | 327 | CG | LYS | A | 20 | 29.443 | 2.034 | 14.166 | 1.00 | 40.05 | C |
| ATOM | 330 | CD | LYS | A | 20 | 28.840 | 0.666 | 13.783 | 1.00 | 49.00 | C |
| ATOM | 333 | CE | LYS | A | 20 | 27.927 | 0.730 | 12.564 | 1.00 | 64.04 | C |
| ATOM | 336 | NZ | LYS | A | 20 | 28.678 | 0.916 | 11.271 | 1.00 | 73.48 | N |
| ATOM | 340 | C | LYS | A | 20 | 31.877 | 3.694 | 14.430 | 1.00 | 52.48 | C |
| ATOM | 341 | O | LYS | A | 20 | 32.890 | 3.171 | 13.959 | 1.00 | 50.59 | O |
| ATOM | 342 | N | ASP | A | 21 | 31.260 | 4.726 | 13.850 | 1.00 | 47.63 | N |
| ATOM | 344 | CA | ASP | A | 21 | 31.801 | 5.303 | 12.624 | 1.00 | 55.30 | C |
| ATOM | 346 | CB | ASP | A | 21 | 30.681 | 5.939 | 11.774 | 1.00 | 55.52 | C |
| ATOM | 349 | CG | ASP | A | 21 | 29.706 | 4.900 | 11.165 | 1.00 | 51.12 | C |
| ATOM | 350 | OD1 | ASP | A | 21 | 30.106 | 3.740 | 10.922 | 1.00 | 70.49 | O |
| ATOM | 351 | OD2 | ASP | A | 21 | 28.508 | 5.156 | 10.895 | 1.00 | 59.68 | O |
| ATOM | 352 | C | ASP | A | 21 | 32.926 | 6.304 | 12.966 | 1.00 | 53.73 | C |
| ATOM | 353 | O | ASP | A | 21 | 33.958 | 6.367 | 12.296 | 1.00 | 63.24 | O |
| ATOM | 354 | N | LEU | A | 22 | 32.767 | 7.010 | 14.074 | 1.00 | 46.21 | N |
| ATOM | 356 | CA | LEU | A | 22 | 33.683 | 8.060 | 14.473 | 1.00 | 46.98 | C |
| ATOM | 358 | CB | LEU | A | 22 | 32.946 | 8.995 | 15.406 | 1.00 | 38.84 | C |
| ATOM | 361 | CG | LEU | A | 22 | 33.800 | 10.128 | 15.935 | 1.00 | 43.32 | C |
| ATOM | 363 | CD1 | LEU | A | 22 | 34.363 | 10.963 | 14.780 | 1.00 | 41.57 | C |
| ATOM | 367 | CD2 | LEU | A | 22 | 32.958 | 10.965 | 16.862 | 1.00 | 37.59 | C |
| ATOM | 371 | C | LEU | A | 22 | 34.938 | 7.557 | 15.190 | 1.00 | 46.37 | C |
| ATOM | 372 | O | LEU | A | 22 | 36.015 | 8.122 | 15.062 | 1.00 | 54.29 | O |
| ATOM | 373 | N | ILE | A | 23 | 34.784 | 6.504 | 15.973 | 1.00 | 52.57 | N |
| ATOM | 375 | CA | ILE | A | 23 | 35.814 | 6.058 | 16.903 | 1.00 | 52.18 | C |
| ATOM | 377 | CB | ILE | A | 23 | 35.366 | 6.380 | 18.340 | 1.00 | 53.29 | C |
| ATOM | 379 | CG1 | ILE | A | 23 | 35.503 | 7.868 | 18.611 | 1.00 | 63.45 | C |
| ATOM | 382 | CD1 | ILE | A | 23 | 34.560 | 8.315 | 19.687 | 1.00 | 73.84 | C |
| ATOM | 386 | CG2 | ILE | A | 23 | 36.148 | 5.589 | 19.367 | 1.00 | 54.48 | C |
| ATOM | 390 | C | ILE | A | 23 | 35.908 | 4.556 | 16.778 | 1.00 | 54.06 | C |
| ATOM | 391 | O | ILE | A | 23 | 34.881 | 3.876 | 16.758 | 1.00 | 50.10 | O |
| ATOM | 392 | N | ASN | A | 24 | 37.131 | 4.042 | 16.763 | 1.00 | 53.43 | N |
| ATOM | 394 | CA | ASN | A | 24 | 37.371 | 2.637 | 16.469 | 1.00 | 55.59 | C |
| ATOM | 396 | CB | ASN | A | 24 | 38.471 | 2.522 | 15.413 | 1.00 | 60.82 | C |
| ATOM | 399 | CG | ASN | A | 24 | 37.937 | 2.179 | 14.045 | 1.00 | 69.76 | C |
| ATOM | 400 | OD1 | ASN | A | 24 | 37.224 | 2.971 | 13.420 | 1.00 | 90.91 | O |
| ATOM | 401 | ND2 | ASN | A | 24 | 38.287 | 0.990 | 13.562 | 1.00 | 88.58 | N |
| ATOM | 404 | C | ASN | A | 24 | 37.797 | 1.869 | 17.715 | 1.00 | 56.21 | C |
| ATOM | 405 | O | ASN | A | 24 | 37.598 | 0.657 | 17.804 | 1.00 | 64.86 | O |
| ATOM | 406 | N | GLU | A | 25 | 38.443 | 2.554 | 18.648 | 1.00 | 54.89 | N |
| ATOM | 408 | CA | GLU | A | 25 | 38.899 | 1.930 | 19.885 | 1.00 | 58.38 | C |
| ATOM | 410 | CB | GLU | A | 25 | 40.300 | 1.349 | 19.733 | 1.00 | 59.51 | C |
| ATOM | 413 | CG | GLU | A | 25 | 41.282 | 2.256 | 19.003 | 1.00 | 78.28 | C |
| ATOM | 416 | CD | GLU | A | 25 | 41.357 | 1.973 | 17.508 | 1.00 | 94.08 | C |
| ATOM | 417 | OE1 | GLU | A | 25 | 41.709 | 0.830 | 17.135 | 1.00 | 93.28 | O |
| ATOM | 418 | OE2 | GLU | A | 25 | 41.074 | 2.893 | 16.704 | 1.00 | 90.96 | O |
| ATOM | 419 | C | GLU | A | 25 | 38.877 | 2.969 | 20.993 | 1.00 | 60.41 | C |
| ATOM | 420 | O | GLU | A | 25 | 39.095 | 4.160 | 20.741 | 1.00 | 63.18 | O |
| ATOM | 421 | N | ALA | A | 26 | 38.533 | 2.537 | 22.202 | 1.00 | 50.24 | N |
| ATOM | 423 | CA | ALA | A | 26 | 38.286 | 3.473 | 23.285 | 1.00 | 47.83 | C |
| ATOM | 425 | CB | ALA | A | 26 | 36.841 | 3.983 | 23.234 | 1.00 | 47.65 | C |
| ATOM | 429 | C | ALA | A | 26 | 38.557 | 2.799 | 24.608 | 1.00 | 48.75 | C |
| ATOM | 430 | O | ALA | A | 26 | 38.470 | 1.581 | 24.748 | 1.00 | 50.03 | O |
| ATOM | 431 | N | CYS | A | 27 | 38.889 | 3.618 | 25.589 | 1.00 | 55.26 | N |
| ATOM | 433 | CA | CYS | A | 27 | 39.053 | 3.161 | 26.952 | 1.00 | 53.99 | C |
| ATOM | 435 | CB | CYS | A | 27 | 39.994 | 4.114 | 27.688 | 1.00 | 62.86 | C |
| ATOM | 438 | SG | CYS | A | 27 | 40.570 | 3.461 | 29.272 | 1.00 | 71.07 | S |
| ATOM | 439 | C | CYS | A | 27 | 37.706 | 3.189 | 27.637 | 1.00 | 50.35 | C |
| ATOM | 440 | O | CYS | A | 27 | 37.009 | 4.203 | 27.593 | 1.00 | 51.96 | O |
| ATOM | 441 | N | TRP | A | 28 | 37.360 | 2.087 | 28.294 | 1.00 | 56.03 | N |
| ATOM | 443 | CA | TRP | A | 28 | 36.161 | 2.004 | 29.130 | 1.00 | 52.68 | C |
| ATOM | 445 | CB | TRP | A | 28 | 35.411 | 0.706 | 28.812 | 1.00 | 48.16 | C |
| ATOM | 448 | CG | TRP | A | 28 | 34.579 | 0.884 | 27.561 | 1.00 | 70.30 | C |
| ATOM | 449 | CD1 | TRP | A | 28 | 35.047 | 1.135 | 26.302 | 1.00 | 80.60 | C |
| ATOM | 451 | NE1 | TRP | A | 28 | 34.003 | 1.306 | 25.427 | 1.00 | 72.30 | N |
| ATOM | 453 | CE2 | TRP | A | 28 | 32.826 | 1.193 | 26.114 | 1.00 | 71.81 | C |
| ATOM | 454 | CD2 | TRP | A | 28 | 33.149 | 0.948 | 27.466 | 1.00 | 69.16 | C |
| ATOM | 455 | CE3 | TRP | A | 28 | 32.104 | 0.783 | 28.380 | 1.00 | 82.00 | C |
| ATOM | 457 | CZ3 | TRP | A | 28 | 30.798 | 0.903 | 27.931 | 1.00 | 92.01 | C |
| ATOM | 459 | CH2 | TRP | A | 28 | 30.515 | 1.163 | 26.583 | 1.00 | 86.45 | C |
| ATOM | 461 | CZ2 | TRP | A | 28 | 31.512 | 1.298 | 25.660 | 1.00 | 77.90 | C |
| ATOM | 463 | C | TRP | A | 28 | 36.506 | 2.100 | 30.614 | 1.00 | 51.70 | C |
| ATOM | 464 | O | TRP | A | 28 | 37.035 | 1.141 | 31.168 | 1.00 | 56.64 | O |
| ATOM | 465 | N | ASP | A | 29 | 36.256 | 3.247 | 31.253 | 1.00 | 48.47 | N |
| ATOM | 467 | CA | ASP | A | 29 | 36.527 | 3.377 | 32.691 | 1.00 | 49.82 | C |
| ATOM | 469 | CB | ASP | A | 29 | 36.920 | 4.807 | 33.131 | 1.00 | 49.80 | C |
| ATOM | 472 | CG | ASP | A | 29 | 38.357 | 5.139 | 32.797 | 1.00 | 64.21 | C |
| ATOM | 473 | OD1 | ASP | A | 29 | 38.728 | 4.926 | 31.623 | 1.00 | 68.60 | O |
| ATOM | 474 | OD2 | ASP | A | 29 | 39.181 | 5.603 | 33.622 | 1.00 | 77.81 | O |
| ATOM | 475 | C | ASP | A | 29 | 35.295 | 2.951 | 33.461 | 1.00 | 39.83 | C |
| ATOM | 476 | O | ASP | A | 29 | 34.299 | 3.640 | 33.474 | 1.00 | 49.36 | O |
| ATOM | 477 | N | ILE | A | 30 | 35.387 | 1.812 | 34.120 | 1.00 | 39.00 | N |
| ATOM | 479 | CA | ILE | A | 30 | 34.313 | 1.325 | 34.948 | 1.00 | 42.93 | C |
| ATOM | 481 | CB | ILE | A | 30 | 34.080 | −0.174 | 34.715 | 1.00 | 46.02 | C |
| ATOM | 483 | CG1 | ILE | A | 30 | 34.248 | −0.560 | 33.245 | 1.00 | 53.08 | C |
| ATOM | 486 | CD1 | ILE | A | 30 | 33.454 | 0.281 | 32.252 | 1.00 | 61.74 | C |
| ATOM | 490 | CG2 | ILE | A | 30 | 32.704 | −0.547 | 35.262 | 1.00 | 49.85 | C |
| ATOM | 494 | C | ILE | A | 30 | 34.617 | 1.523 | 36.422 | 1.00 | 37.72 | C |
| ATOM | 495 | O | ILE | A | 30 | 35.683 | 1.143 | 36.896 | 1.00 | 47.64 | O |
| ATOM | 496 | N | SER | A | 31 | 33.637 | 2.039 | 37.150 | 1.00 | 40.90 | N |
| ATOM | 498 | CA | SER | A | 31 | 33.723 | 2.258 | 38.586 | 1.00 | 40.50 | C |
| ATOM | 500 | CB | SER | A | 31 | 34.108 | 3.723 | 38.881 | 1.00 | 44.13 | C |
| ATOM | 503 | OG | SER | A | 31 | 32.991 | 4.593 | 38.743 | 1.00 | 42.55 | O |
| ATOM | 505 | C | SER | A | 31 | 32.355 | 1.964 | 39.206 | 1.00 | 47.40 | C |
| ATOM | 506 | O | SER | A | 31 | 31.386 | 1.645 | 38.509 | 1.00 | 44.22 | O |
| ATOM | 507 | N | SER | A | 32 | 32.265 | 2.114 | 40.525 | 1.00 | 51.60 | N |
| ATOM | 509 | CA | SER | A | 32 | 31.036 | 1.791 | 41.238 | 1.00 | 48.74 | C |
| ATOM | 511 | CB | SER | A | 32 | 31.282 | 1.574 | 42.738 | 1.00 | 53.60 | C |
| ATOM | 514 | OG | SER | A | 32 | 32.035 | 2.637 | 43.311 | 1.00 | 52.20 | O |
| ATOM | 516 | C | SER | A | 32 | 29.995 | 2.872 | 41.006 | 1.00 | 44.88 | C |
| ATOM | 517 | O | SER | A | 32 | 28.806 | 2.634 | 41.181 | 1.00 | 41.51 | O |
| ATOM | 518 | N | SER | A | 33 | 30.409 | 4.033 | 40.527 | 1.00 | 47.23 | N |
| ATOM | 520 | CA | SER | A | 33 | 29.430 | 5.053 | 40.155 | 1.00 | 51.40 | C |
| ATOM | 522 | CB | SER | A | 33 | 29.927 | 6.447 | 40.549 | 1.00 | 52.19 | C |
| ATOM | 525 | OG | SER | A | 33 | 30.606 | 7.038 | 39.469 | 1.00 | 60.37 | O |
| ATOM | 527 | C | SER | A | 33 | 29.035 | 4.992 | 38.667 | 1.00 | 49.26 | C |
| ATOM | 528 | O | SER | A | 33 | 28.023 | 5.573 | 38.262 | 1.00 | 51.04 | O |
| ATOM | 529 | N | GLY | A | 34 | 29.794 | 4.242 | 37.874 | 1.00 | 48.99 | N |
| ATOM | 531 | CA | GLY | A | 34 | 29.329 | 3.799 | 36.568 | 1.00 | 45.88 | C |
| ATOM | 534 | C | GLY | A | 34 | 30.354 | 3.758 | 35.439 | 1.00 | 46.13 | C |
| ATOM | 535 | O | GLY | A | 34 | 31.588 | 3.672 | 35.654 | 1.00 | 41.60 | O |
| ATOM | 536 | N | VAL | A | 35 | 29.825 | 3.921 | 34.221 | 1.00 | 39.55 | N |
| ATOM | 538 | CA | VAL | A | 35 | 30.620 | 3.801 | 33.007 | 1.00 | 41.00 | C |
| ATOM | 540 | CB | VAL | A | 35 | 29.970 | 2.887 | 31.969 | 1.00 | 44.87 | C |
| ATOM | 542 | CG1 | VAL | A | 35 | 30.812 | 2.866 | 30.682 | 1.00 | 51.05 | C |
| ATOM | 546 | CG2 | VAL | A | 35 | 29.790 | 1.475 | 32.523 | 1.00 | 39.58 | C |
| ATOM | 550 | C | VAL | A | 35 | 30.912 | 5.133 | 32.357 | 1.00 | 45.83 | C |
| ATOM | 551 | O | VAL | A | 35 | 29.997 | 5.916 | 32.085 | 1.00 | 45.69 | O |
| ATOM | 552 | N | ASN | A | 36 | 32.192 | 5.362 | 32.067 | 1.00 | 38.52 | N |
| ATOM | 554 | CA | ASN | A | 36 | 32.641 | 6.651 | 31.532 | 1.00 | 54.56 | C |
| ATOM | 556 | CB | ASN | A | 36 | 33.360 | 7.460 | 32.634 | 1.00 | 50.00 | C |
| ATOM | 559 | CG | ASN | A | 36 | 32.395 | 8.159 | 33.594 | 1.00 | 59.25 | C |
| ATOM | 560 | OD1 | ASN | A | 36 | 31.575 | 8.992 | 33.192 | 1.00 | 64.42 | O |
| ATOM | 561 | ND2 | ASN | A | 36 | 32.507 | 7.836 | 34.880 | 1.00 | 77.43 | N |
| ATOM | 564 | C | ASN | A | 36 | 33.626 | 6.425 | 30.376 | 1.00 | 54.75 | C |
| ATOM | 565 | O | ASN | A | 36 | 34.754 | 6.009 | 30.621 | 1.00 | 61.97 | O |
| ATOM | 566 | N | LEU | A | 37 | 33.235 | 6.695 | 29.136 | 1.00 | 55.93 | N |
| ATOM | 568 | CA | LEU | A | 37 | 34.153 | 6.514 | 27.999 | 1.00 | 57.09 | C |
| ATOM | 570 | CB | LEU | A | 37 | 33.470 | 5.706 | 26.914 | 1.00 | 54.42 | C |
| ATOM | 573 | CG | LEU | A | 37 | 34.277 | 5.618 | 25.629 | 1.00 | 56.27 | C |
| ATOM | 575 | CD1 | LEU | A | 37 | 33.651 | 4.560 | 24.749 | 1.00 | 59.33 | C |
| ATOM | 579 | CD2 | LEU | A | 37 | 34.328 | 6.956 | 24.912 | 1.00 | 62.69 | C |
| ATOM | 583 | C | LEU | A | 37 | 34.607 | 7.850 | 27.420 | 1.00 | 58.09 | C |
| ATOM | 584 | O | LEU | A | 37 | 33.829 | 8.810 | 27.445 | 1.00 | 61.52 | O |
| ATOM | 585 | N | GLN | A | 38 | 35.836 | 7.917 | 26.892 | 1.00 | 58.13 | N |
| ATOM | 587 | CA | GLN | A | 38 | 36.400 | 9.183 | 26.382 | 1.00 | 61.54 | C |
| ATOM | 589 | CB | GLN | A | 38 | 36.956 | 10.027 | 27.524 | 1.00 | 55.07 | C |
| ATOM | 592 | CG | GLN | A | 38 | 36.725 | 11.513 | 27.338 | 1.00 | 56.15 | C |
| ATOM | 595 | CD | GLN | A | 38 | 37.381 | 12.354 | 28.424 | 1.00 | 75.80 | C |
| ATOM | 596 | OE1 | GLN | A | 38 | 37.695 | 13.527 | 28.210 | 1.00 | 77.01 | O |
| ATOM | 597 | NE2 | GLN | A | 38 | 37.577 | 11.760 | 29.596 | 1.00 | 90.64 | N |
| ATOM | 600 | C | GLN | A | 38 | 37.487 | 9.073 | 25.309 | 1.00 | 67.39 | C |
| ATOM | 601 | O | GLN | A | 38 | 38.597 | 8.657 | 25.608 | 1.00 | 73.90 | O |
| ATOM | 602 | N | SER | A | 39 | 37.219 | 9.519 | 24.081 | 1.00 | 68.55 | N |
| ATOM | 604 | CA | SER | A | 39 | 38.163 | 9.266 | 22.997 | 1.00 | 63.53 | C |
| ATOM | 606 | CB | SER | A | 39 | 37.868 | 7.901 | 22.404 | 1.00 | 63.14 | C |
| ATOM | 609 | OG | SER | A | 39 | 38.870 | 7.576 | 21.466 | 1.00 | 69.17 | O |
| ATOM | 611 | C | SER | A | 39 | 38.195 | 10.291 | 21.863 | 1.00 | 60.13 | C |
| ATOM | 612 | O | SER | A | 39 | 37.171 | 10.864 | 21.528 | 1.00 | 63.31 | O |
| ATOM | 613 | N | MET | A | 40 | 39.374 | 10.468 | 21.263 | 1.00 | 59.18 | N |
| ATOM | 615 | CA | MET | A | 40 | 39.561 | 11.188 | 20.003 | 1.00 | 56.70 | C |
| ATOM | 617 | CB | MET | A | 40 | 40.959 | 11.805 | 19.931 | 1.00 | 56.04 | C |
| ATOM | 620 | CG | MET | A | 40 | 41.092 | 13.126 | 20.656 | 1.00 | 72.87 | C |
| ATOM | 623 | SD | MET | A | 40 | 42.522 | 14.067 | 20.085 | 1.00 | 93.46 | S |
| ATOM | 624 | CE | MET | A | 40 | 41.703 | 15.535 | 19.418 | 1.00 | 89.23 | C |
| ATOM | 628 | C | MET | A | 40 | 39.386 | 10.289 | 18.780 | 1.00 | 48.74 | C |
| ATOM | 629 | O | MET | A | 40 | 39.660 | 9.106 | 18.830 | 1.00 | 59.42 | O |
| ATOM | 630 | N | ASP | A | 41 | 38.876 | 10.861 | 17.693 | 1.00 | 45.98 | N |
| ATOM | 632 | CA | ASP | A | 41 | 38.809 | 10.180 | 16.404 | 1.00 | 54.25 | C |
| ATOM | 634 | CB | ASP | A | 41 | 37.922 | 10.969 | 15.423 | 1.00 | 53.46 | C |
| ATOM | 637 | CG | ASP | A | 41 | 38.646 | 12.168 | 14.786 | 1.00 | 60.35 | C |
| ATOM | 638 | OD1 | ASP | A | 41 | 39.048 | 12.087 | 13.608 | 1.00 | 47.52 | O |
| ATOM | 639 | OD2 | ASP | A | 41 | 38.831 | 13.258 | 15.359 | 1.00 | 52.18 | O |
| ATOM | 640 | C | ASP | A | 41 | 40.212 | 10.020 | 15.812 | 1.00 | 53.16 | C |
| ATOM | 641 | O | ASP | A | 41 | 41.124 | 10.774 | 16.151 | 1.00 | 44.78 | O |
| ATOM | 642 | N | SER | A | 42 | 40.380 | 9.040 | 14.932 | 1.00 | 60.93 | N |
| ATOM | 644 | CA | SER | A | 42 | 41.670 | 8.810 | 14.276 | 1.00 | 65.99 | C |
| ATOM | 646 | CB | SER | A | 42 | 41.746 | 7.423 | 13.621 | 1.00 | 65.90 | C |
| ATOM | 649 | OG | SER | A | 42 | 40.450 | 6.861 | 13.378 | 1.00 | 89.49 | O |
| ATOM | 651 | C | SER | A | 42 | 41.881 | 9.894 | 13.220 | 1.00 | 73.54 | C |
| ATOM | 652 | O | SER | A | 42 | 41.728 | 9.642 | 12.017 | 1.00 | 85.22 | O |
| ATOM | 653 | N | SER | A | 43 | 42.141 | 11.109 | 13.701 | 1.00 | 70.47 | N |
| ATOM | 655 | CA | SER | A | 43 | 42.523 | 12.265 | 12.890 | 1.00 | 67.52 | C |
| ATOM | 657 | CB | SER | A | 43 | 41.648 | 12.457 | 11.642 | 1.00 | 64.56 | C |
| ATOM | 660 | OG | SER | A | 43 | 40.376 | 11.833 | 11.790 | 1.00 | 91.00 | O |
| ATOM | 662 | C | SER | A | 43 | 42.532 | 13.526 | 13.761 | 1.00 | 57.91 | C |
| ATOM | 663 | O | SER | A | 43 | 42.926 | 14.599 | 13.300 | 1.00 | 53.29 | O |
| ATOM | 664 | N | HIS | A | 44 | 42.149 | 13.395 | 15.028 | 1.00 | 49.39 | N |
| ATOM | 666 | CA | HIS | A | 44 | 42.457 | 14.435 | 16.026 | 1.00 | 50.87 | C |
| ATOM | 668 | CB | HIS | A | 44 | 43.981 | 14.658 | 16.030 | 1.00 | 56.16 | C |
| ATOM | 671 | CG | HIS | A | 44 | 44.770 | 13.379 | 16.020 | 1.00 | 49.43 | C |
| ATOM | 672 | ND1 | HIS | A | 44 | 45.869 | 13.177 | 15.212 | 1.00 | 51.97 | N |
| ATOM | 674 | CE1 | HIS | A | 44 | 46.330 | 11.952 | 15.398 | 1.00 | 50.82 | C |
| ATOM | 676 | NE2 | HIS | A | 44 | 45.522 | 11.325 | 16.231 | 1.00 | 59.95 | N |
| ATOM | 678 | CD2 | HIS | A | 44 | 44.556 | 12.205 | 16.661 | 1.00 | 49.69 | C |
| ATOM | 680 | C | HIS | A | 44 | 41.710 | 15.774 | 15.859 | 1.00 | 46.23 | C |
| ATOM | 681 | O | HIS | A | 44 | 42.236 | 16.834 | 16.187 | 1.00 | 44.40 | O |
| ATOM | 682 | N | VAL | A | 45 | 40.491 | 15.739 | 15.327 | 1.00 | 49.66 | N |
| ATOM | 684 | CA | VAL | A | 45 | 39.658 | 16.940 | 15.247 | 1.00 | 46.64 | C |
| ATOM | 686 | CB | VAL | A | 45 | 39.012 | 17.087 | 13.860 | 1.00 | 49.33 | C |
| ATOM | 688 | CG1 | VAL | A | 45 | 38.323 | 18.429 | 13.754 | 1.00 | 52.49 | C |
| ATOM | 692 | CG2 | VAL | A | 45 | 40.068 | 16.967 | 12.737 | 1.00 | 55.57 | C |
| ATOM | 696 | C | VAL | A | 45 | 38.562 | 16.944 | 16.325 | 1.00 | 46.05 | C |
| ATOM | 697 | O | VAL | A | 45 | 38.108 | 18.004 | 16.772 | 1.00 | 43.72 | O |
| ATOM | 698 | N | SER | A | 46 | 38.118 | 15.761 | 16.735 | 1.00 | 34.32 | N |
| ATOM | 700 | CA | SER | A | 46 | 36.928 | 15.668 | 17.561 | 1.00 | 34.16 | C |
| ATOM | 702 | CB | SER | A | 46 | 35.775 | 15.022 | 16.787 | 1.00 | 31.91 | C |
| ATOM | 705 | OG | SER | A | 46 | 36.064 | 13.632 | 16.684 | 1.00 | 36.65 | O |
| ATOM | 707 | C | SER | A | 46 | 37.216 | 14.790 | 18.745 | 1.00 | 36.49 | C |
| ATOM | 708 | O | SER | A | 46 | 38.055 | 13.899 | 18.667 | 1.00 | 39.56 | O |
| ATOM | 709 | N | LEU | A | 47 | 36.395 | 14.957 | 19.780 | 1.00 | 43.85 | N |
| ATOM | 711 | CA | LEU | A | 47 | 36.449 | 14.133 | 20.984 | 1.00 | 39.47 | C |
| ATOM | 713 | CB | LEU | A | 47 | 37.195 | 14.901 | 22.060 | 1.00 | 34.56 | C |
| ATOM | 716 | CG | LEU | A | 47 | 37.601 | 14.230 | 23.361 | 1.00 | 51.24 | C |
| ATOM | 718 | CD1 | LEU | A | 47 | 39.130 | 14.247 | 23.441 | 1.00 | 47.79 | C |
| ATOM | 722 | CD2 | LEU | A | 47 | 36.972 | 14.984 | 24.538 | 1.00 | 49.17 | C |
| ATOM | 726 | C | LEU | A | 47 | 35.042 | 13.790 | 21.497 | 1.00 | 42.75 | C |
| ATOM | 727 | O | LEU | A | 47 | 34.188 | 14.667 | 21.629 | 1.00 | 40.38 | O |
| ATOM | 728 | N | VAL | A | 48 | 34.839 | 12.519 | 21.820 | 1.00 | 44.69 | N |
| ATOM | 730 | CA | VAL | A | 48 | 33.595 | 12.034 | 22.394 | 1.00 | 45.78 | C |
| ATOM | 732 | CB | VAL | A | 48 | 33.136 | 10.741 | 21.712 | 1.00 | 43.84 | C |
| ATOM | 734 | CG1 | VAL | A | 48 | 31.783 | 10.308 | 22.264 | 1.00 | 54.41 | C |
| ATOM | 738 | CG2 | VAL | A | 48 | 33.041 | 10.967 | 20.222 | 1.00 | 50.15 | C |
| ATOM | 742 | C | VAL | A | 48 | 33.773 | 11.662 | 23.850 | 1.00 | 43.84 | C |
| ATOM | 743 | O | VAL | A | 48 | 34.773 | 11.056 | 24.231 | 1.00 | 54.28 | O |
| ATOM | 744 | N | GLN | A | 49 | 32.739 | 11.924 | 24.632 | 1.00 | 36.25 | N |
| ATOM | 746 | CA | GLN | A | 49 | 32.725 | 11.595 | 26.039 | 1.00 | 37.76 | C |
| ATOM | 748 | CB | GLN | A | 49 | 33.014 | 12.866 | 26.837 | 1.00 | 41.71 | C |
| ATOM | 751 | CG | GLN | A | 49 | 33.358 | 12.626 | 28.295 | 1.00 | 60.14 | C |
| ATOM | 754 | CD | GLN | A | 49 | 32.213 | 12.927 | 29.244 | 1.00 | 63.83 | C |
| ATOM | 755 | OE1 | GLN | A | 49 | 31.627 | 14.013 | 29.220 | 1.00 | 72.13 | O |
| ATOM | 756 | NE2 | GLN | A | 49 | 31.948 | 11.987 | 30.137 | 1.00 | 34.54 | N |
| ATOM | 759 | C | GLN | A | 49 | 31.360 | 11.021 | 26.431 | 1.00 | 36.68 | C |
| ATOM | 760 | O | GLN | A | 49 | 30.343 | 11.720 | 26.397 | 1.00 | 35.03 | O |
| ATOM | 761 | N | LEU | A | 50 | 31.351 | 9.741 | 26.790 | 1.00 | 32.49 | N |
| ATOM | 763 | CA | LEU | A | 50 | 30.134 | 9.015 | 27.137 | 1.00 | 34.07 | C |
| ATOM | 765 | CB | LEU | A | 50 | 30.056 | 7.714 | 26.334 | 1.00 | 32.26 | C |
| ATOM | 768 | CG | LEU | A | 50 | 28.988 | 6.791 | 26.895 | 1.00 | 48.00 | C |
| ATOM | 770 | CD1 | LEU | A | 50 | 27.605 | 7.318 | 26.484 | 1.00 | 26.42 | C |
| ATOM | 774 | CD2 | LEU | A | 50 | 29.220 | 5.358 | 26.430 | 1.00 | 46.62 | C |
| ATOM | 778 | C | LEU | A | 50 | 30.074 | 8.688 | 28.650 | 1.00 | 38.99 | C |
| ATOM | 779 | O | LEU | A | 50 | 31.077 | 8.296 | 29.264 | 1.00 | 35.37 | O |
| ATOM | 780 | N | THR | A | 51 | 28.896 | 8.905 | 29.242 | 1.00 | 35.51 | N |
| ATOM | 782 | CA | THR | A | 51 | 28.628 | 8.623 | 30.651 | 1.00 | 29.69 | C |
| ATOM | 784 | CB | THR | A | 51 | 28.718 | 9.916 | 31.488 | 1.00 | 37.17 | C |
| ATOM | 786 | OG1 | THR | A | 51 | 30.035 | 10.461 | 31.409 | 1.00 | 43.58 | O |
| ATOM | 788 | CG2 | THR | A | 51 | 28.601 | 9.609 | 32.930 | 1.00 | 31.49 | C |
| ATOM | 792 | C | THR | A | 51 | 27.287 | 7.902 | 30.903 | 1.00 | 32.02 | C |
| ATOM | 793 | O | THR | A | 51 | 26.220 | 8.381 | 30.497 | 1.00 | 40.13 | O |
| ATOM | 794 | N | LEU | A | 52 | 27.387 | 6.729 | 31.526 | 1.00 | 31.34 | N |
| ATOM | 796 | CA | LEU | A | 52 | 26.268 | 5.880 | 31.930 | 1.00 | 27.07 | C |
| ATOM | 798 | CB | LEU | A | 52 | 26.298 | 4.560 | 31.198 | 1.00 | 22.61 | C |
| ATOM | 801 | CG | LEU | A | 52 | 26.192 | 4.585 | 29.692 | 1.00 | 34.70 | C |
| ATOM | 803 | CD1 | LEU | A | 52 | 26.177 | 3.165 | 29.229 | 1.00 | 35.07 | C |
| ATOM | 807 | CD2 | LEU | A | 52 | 24.915 | 5.348 | 29.334 | 1.00 | 53.20 | C |
| ATOM | 811 | C | LEU | A | 52 | 26.315 | 5.585 | 33.440 | 1.00 | 38.33 | C |
| ATOM | 812 | O | LEU | A | 52 | 27.226 | 4.888 | 33.916 | 1.00 | 36.67 | O |
| ATOM | 813 | N | ARG | A | 53 | 25.325 | 6.094 | 34.188 | 1.00 | 35.44 | N |
| ATOM | 815 | CA | ARG | A | 53 | 25.390 | 6.045 | 35.640 | 1.00 | 27.63 | C |
| ATOM | 817 | CB | ARG | A | 53 | 24.527 | 7.118 | 36.285 | 1.00 | 35.27 | C |
| ATOM | 820 | CG | ARG | A | 53 | 25.061 | 8.521 | 36.154 | 1.00 | 32.68 | C |
| ATOM | 823 | CD | ARG | A | 53 | 24.197 | 9.585 | 36.848 | 1.00 | 47.29 | C |
| ATOM | 826 | NE | ARG | A | 53 | 23.279 | 10.212 | 35.899 | 1.00 | 57.30 | N |
| ATOM | 828 | CZ | ARG | A | 53 | 22.006 | 9.877 | 35.761 | 1.00 | 59.80 | C |
| ATOM | 829 | NH1 | ARG | A | 53 | 21.466 | 8.940 | 36.545 | 1.00 | 70.17 | N |
| ATOM | 832 | NH2 | ARG | A | 53 | 21.272 | 10.468 | 34.823 | 1.00 | 53.98 | N |
| ATOM | 835 | C | ARG | A | 53 | 24.939 | 4.675 | 36.068 | 1.00 | 34.21 | C |
| ATOM | 836 | O | ARG | A | 53 | 24.032 | 4.098 | 35.456 | 1.00 | 29.91 | O |
| ATOM | 837 | N | SER | A | 54 | 25.516 | 4.217 | 37.179 | 1.00 | 33.86 | N |
| ATOM | 839 | CA | SER | A | 54 | 25.222 | 2.915 | 37.731 | 1.00 | 33.50 | C |
| ATOM | 841 | CB | SER | A | 54 | 26.047 | 2.701 | 38.994 | 1.00 | 39.00 | C |
| ATOM | 844 | OG | SER | A | 54 | 26.111 | 3.894 | 39.742 | 1.00 | 44.01 | O |
| ATOM | 846 | C | SER | A | 54 | 23.751 | 2.745 | 38.067 | 1.00 | 39.96 | C |
| ATOM | 847 | O | SER | A | 54 | 23.196 | 1.662 | 37.893 | 1.00 | 36.59 | O |
| ATOM | 848 | N | GLU | A | 55 | 23.140 | 3.824 | 38.547 | 1.00 | 41.44 | N |
| ATOM | 850 | CA | GLU | A | 55 | 21.737 | 3.823 | 38.942 | 1.00 | 41.68 | C |
| ATOM | 852 | CB | GLU | A | 55 | 21.430 | 5.122 | 39.678 | 1.00 | 46.58 | C |
| ATOM | 855 | CG | GLU | A | 55 | 22.333 | 5.358 | 40.882 | 1.00 | 51.56 | C |
| ATOM | 858 | CD | GLU | A | 55 | 23.437 | 6.365 | 40.615 | 1.00 | 59.10 | C |
| ATOM | 859 | OE1 | GLU | A | 55 | 23.479 | 6.915 | 39.497 | 1.00 | 64.94 | O |
| ATOM | 860 | OE2 | GLU | A | 55 | 24.253 | 6.621 | 41.525 | 1.00 | 66.67 | O |
| ATOM | 861 | C | GLU | A | 55 | 20.832 | 3.732 | 37.736 | 1.00 | 44.18 | C |
| ATOM | 862 | O | GLU | A | 55 | 19.635 | 3.457 | 37.878 | 1.00 | 51.07 | O |
| ATOM | 863 | N | GLY | A | 56 | 21.399 | 4.004 | 36.562 | 1.00 | 40.89 | N |
| ATOM | 865 | CA | GLY | A | 56 | 20.696 | 3.798 | 35.303 | 1.00 | 47.65 | C |
| ATOM | 868 | C | GLY | A | 56 | 20.476 | 2.347 | 34.880 | 1.00 | 43.52 | C |
| ATOM | 869 | O | GLY | A | 56 | 19.603 | 2.047 | 34.078 | 1.00 | 43.16 | O |
| ATOM | 870 | N | PHE | A | 57 | 21.308 | 1.452 | 35.403 | 1.00 | 38.79 | N |
| ATOM | 872 | CA | PHE | A | 57 | 21.189 | 0.031 | 35.177 | 1.00 | 35.21 | C |
| ATOM | 874 | CB | PHE | A | 57 | 22.572 | −0.575 | 35.388 | 1.00 | 41.88 | C |
| ATOM | 877 | CG | PHE | A | 57 | 23.558 | −0.169 | 34.311 | 1.00 | 37.34 | C |
| ATOM | 878 | CD1 | PHE | A | 57 | 24.223 | 1.038 | 34.388 | 1.00 | 33.02 | C |
| ATOM | 880 | CE1 | PHE | A | 57 | 25.070 | 1.453 | 33.394 | 1.00 | 39.28 | C |
| ATOM | 882 | CZ | PHE | A | 57 | 25.242 | 0.675 | 32.267 | 1.00 | 33.18 | C |
| ATOM | 884 | CE2 | PHE | A | 57 | 24.514 | −0.484 | 32.123 | 1.00 | 29.66 | C |
| ATOM | 886 | CD2 | PHE | A | 57 | 23.673 | −0.907 | 33.151 | 1.00 | 36.16 | C |
| ATOM | 888 | C | PHE | A | 57 | 20.151 | −0.558 | 36.111 | 1.00 | 40.25 | C |
| ATOM | 889 | O | PHE | A | 57 | 19.820 | 0.031 | 37.142 | 1.00 | 45.47 | O |
| ATOM | 890 | N | ASP | A | 58 | 19.570 | −1.669 | 35.698 | 1.00 | 41.80 | N |
| ATOM | 892 | CA | ASP | A | 58 | 18.762 | −2.474 | 36.592 | 1.00 | 49.18 | C |
| ATOM | 894 | CB | ASP | A | 58 | 17.978 | −3.538 | 35.811 | 1.00 | 45.22 | C |
| ATOM | 897 | CG | ASP | A | 58 | 16.514 | −3.160 | 35.661 | 1.00 | 69.29 | C |
| ATOM | 898 | OD1 | ASP | A | 58 | 16.218 | −2.084 | 35.075 | 1.00 | 58.68 | O |
| ATOM | 899 | OD2 | ASP | A | 58 | 15.597 | −3.841 | 36.174 | 1.00 | 87.64 | O |
| ATOM | 900 | C | ASP | A | 58 | 19.571 | −3.116 | 37.715 | 1.00 | 53.44 | C |
| ATOM | 901 | O | ASP | A | 58 | 19.095 | −3.208 | 38.842 | 1.00 | 64.71 | O |
| ATOM | 902 | N | THR | A | 59 | 20.758 | −3.621 | 37.411 | 1.00 | 49.74 | N |
| ATOM | 904 | CA | THR | A | 59 | 21.630 | −4.109 | 38.463 | 1.00 | 51.18 | C |
| ATOM | 906 | CB | THR | A | 59 | 21.633 | −5.639 | 38.537 | 1.00 | 52.27 | C |
| ATOM | 908 | OG1 | THR | A | 59 | 20.327 | −6.134 | 38.241 | 1.00 | 70.72 | O |
| ATOM | 910 | CG2 | THR | A | 59 | 21.857 | −6.095 | 39.947 | 1.00 | 62.18 | C |
| ATOM | 914 | C | THR | A | 59 | 23.007 | −3.627 | 38.124 | 1.00 | 49.11 | C |
| ATOM | 915 | O | THR | A | 59 | 23.356 | −3.568 | 36.941 | 1.00 | 43.58 | O |
| ATOM | 916 | N | TYR | A | 60 | 23.793 | −3.320 | 39.152 | 1.00 | 40.49 | N |
| ATOM | 918 | CA | TYR | A | 60 | 25.158 | −2.880 | 38.905 | 1.00 | 43.18 | C |
| ATOM | 920 | CB | TYR | A | 60 | 25.164 | −1.365 | 38.738 | 1.00 | 43.60 | C |
| ATOM | 923 | CG | TYR | A | 60 | 26.441 | −0.822 | 38.154 | 1.00 | 39.81 | C |
| ATOM | 924 | CD1 | TYR | A | 60 | 26.551 | −0.604 | 36.781 | 1.00 | 34.23 | C |
| ATOM | 926 | CE1 | TYR | A | 60 | 27.710 | −0.087 | 36.224 | 1.00 | 25.95 | C |
| ATOM | 928 | CZ | TYR | A | 60 | 28.784 | 0.186 | 37.037 | 1.00 | 37.51 | C |
| ATOM | 929 | OH | TYR | A | 60 | 29.902 | 0.710 | 36.438 | 1.00 | 32.15 | O |
| ATOM | 931 | CE2 | TYR | A | 60 | 28.695 | 0.002 | 38.428 | 1.00 | 32.91 | C |
| ATOM | 933 | CD2 | TYR | A | 60 | 27.517 | −0.484 | 38.968 | 1.00 | 34.50 | C |
| ATOM | 935 | C | TYR | A | 60 | 26.080 | −3.253 | 40.047 | 1.00 | 43.10 | C |
| ATOM | 936 | O | TYR | A | 60 | 25.785 | −2.989 | 41.199 | 1.00 | 54.30 | O |
| ATOM | 937 | N | ARG | A | 61 | 27.223 | −3.825 | 39.729 | 1.00 | 45.12 | N |
| ATOM | 939 | CA | ARG | A | 61 | 28.230 | −4.057 | 40.739 | 1.00 | 46.85 | C |
| ATOM | 941 | CB | ARG | A | 61 | 28.109 | −5.469 | 41.299 | 1.00 | 52.16 | C |
| ATOM | 944 | CG | ARG | A | 61 | 29.252 | −5.836 | 42.265 | 1.00 | 74.19 | C |
| ATOM | 947 | CD | ARG | A | 61 | 30.025 | −7.115 | 41.897 | 1.00 | 90.10 | C |
| ATOM | 950 | NE | ARG | A | 61 | 30.928 | −7.552 | 42.965 | 1.00 | 93.26 | N |
| ATOM | 952 | CZ | ARG | A | 61 | 31.381 | −8.798 | 43.110 | 1.00 | 90.72 | C |
| ATOM | 953 | NH1 | ARG | A | 61 | 30.995 | −9.760 | 42.280 | 1.00 | 103.25 | N |
| ATOM | 956 | NH2 | ARG | A | 61 | 32.212 | −9.090 | 44.103 | 1.00 | 67.17 | N |
| ATOM | 959 | C | ARG | A | 61 | 29.593 | −3.889 | 40.116 | 1.00 | 44.12 | C |
| ATOM | 960 | O | ARG | A | 61 | 29.878 | −4.475 | 39.075 | 1.00 | 52.41 | O |
| ATOM | 961 | N | CYS | A | 62 | 30.437 | −3.101 | 40.764 | 1.00 | 45.61 | N |
| ATOM | 963 | CA | CYS | A | 62 | 31.848 | −3.021 | 40.404 | 1.00 | 46.98 | C |
| ATOM | 965 | CB | CYS | A | 62 | 32.123 | −1.697 | 39.709 | 1.00 | 45.06 | C |
| ATOM | 968 | SG | CYS | A | 62 | 33.769 | −1.605 | 38.964 | 1.00 | 50.13 | S |
| ATOM | 969 | C | CYS | A | 62 | 32.788 | −3.107 | 41.606 | 1.00 | 46.15 | C |
| ATOM | 970 | O | CYS | A | 62 | 33.013 | −2.108 | 42.284 | 1.00 | 47.15 | O |
| ATOM | 971 | N | ASP | A | 63 | 33.428 | −4.265 | 41.762 | 1.00 | 54.27 | N |
| ATOM | 973 | CA | ASP | A | 63 | 34.448 | −4.516 | 42.785 | 1.00 | 56.46 | C |
| ATOM | 975 | CB | ASP | A | 63 | 34.901 | −5.972 | 42.720 | 1.00 | 56.15 | C |
| ATOM | 978 | CG | ASP | A | 63 | 34.047 | −6.857 | 43.555 | 1.00 | 56.62 | C |
| ATOM | 979 | OD1 | ASP | A | 63 | 32.899 | −6.434 | 43.804 | 1.00 | 87.47 | O |
| ATOM | 980 | OD2 | ASP | A | 63 | 34.437 | −7.930 | 44.063 | 1.00 | 51.27 | O |
| ATOM | 981 | C | ASP | A | 63 | 35.706 | −3.675 | 42.688 | 1.00 | 57.04 | C |
| ATOM | 982 | O | ASP | A | 63 | 36.463 | −3.618 | 43.641 | 1.00 | 56.86 | O |
| ATOM | 983 | N | ARG | A | 64 | 35.926 | −3.010 | 41.563 | 1.00 | 62.24 | N |
| ATOM | 985 | CA | ARG | A | 64 | 37.281 | −2.680 | 41.168 | 1.00 | 67.05 | C |
| ATOM | 987 | CB | ARG | A | 64 | 37.968 | −3.985 | 40.760 | 1.00 | 72.08 | C |
| ATOM | 990 | CG | ARG | A | 64 | 39.468 | −3.883 | 40.579 | 1.00 | 77.09 | C |
| ATOM | 993 | CD | ARG | A | 64 | 40.191 | −5.041 | 41.229 | 1.00 | 84.71 | C |
| ATOM | 996 | NE | ARG | A | 64 | 40.435 | −6.170 | 40.331 | 1.00 | 72.43 | N |
| ATOM | 998 | CZ | ARG | A | 64 | 41.610 | −6.417 | 39.763 | 1.00 | 84.28 | C |
| ATOM | 999 | NH1 | ARG | A | 64 | 42.624 | −5.576 | 39.929 | 1.00 | 93.57 | N |
| ATOM | 1002 | NH2 | ARG | A | 64 | 41.772 | −7.498 | 39.008 | 1.00 | 92.54 | N |
| ATOM | 1005 | C | ARG | A | 64 | 37.295 | −1.746 | 39.975 | 1.00 | 67.01 | C |
| ATOM | 1006 | O | ARG | A | 64 | 36.948 | −2.187 | 38.882 | 1.00 | 67.36 | O |
| ATOM | 1007 | N | ASN | A | 65 | 37.725 | −0.498 | 40.170 | 1.00 | 67.84 | N |
| ATOM | 1009 | CA | ASN | A | 65 | 38.027 | 0.393 | 39.054 | 1.00 | 69.55 | C |
| ATOM | 1011 | CB | ASN | A | 65 | 38.789 | 1.645 | 39.512 | 1.00 | 72.68 | C |
| ATOM | 1014 | CG | ASN | A | 65 | 38.904 | 2.722 | 38.413 | 1.00 | 81.14 | C |
| ATOM | 1015 | OD1 | ASN | A | 65 | 37.926 | 3.395 | 38.080 | 1.00 | 86.14 | O |
| ATOM | 1016 | ND2 | ASN | A | 65 | 40.106 | 2.899 | 37.869 | 1.00 | 82.88 | N |
| ATOM | 1019 | C | ASN | A | 65 | 38.839 | −0.363 | 38.014 | 1.00 | 72.86 | C |
| ATOM | 1020 | O | ASN | A | 65 | 39.667 | −1.210 | 38.344 | 1.00 | 80.66 | O |
| ATOM | 1021 | N | LEU | A | 66 | 38.587 | −0.046 | 36.751 | 1.00 | 76.73 | N |
| ATOM | 1023 | CA | LEU | A | 66 | 39.100 | −0.809 | 35.624 | 1.00 | 72.01 | C |
| ATOM | 1025 | CB | LEU | A | 66 | 38.105 | −1.910 | 35.254 | 1.00 | 73.18 | C |
| ATOM | 1028 | CG | LEU | A | 66 | 38.218 | −3.275 | 35.923 | 1.00 | 79.94 | C |
| ATOM | 1030 | CD1 | LEU | A | 66 | 37.449 | −4.296 | 35.076 | 1.00 | 77.50 | C |
| ATOM | 1034 | CD2 | LEU | A | 66 | 39.687 | −3.678 | 36.115 | 1.00 | 83.88 | C |
| ATOM | 1038 | C | LEU | A | 66 | 39.169 | 0.141 | 34.443 | 1.00 | 66.47 | C |
| ATOM | 1039 | O | LEU | A | 66 | 38.263 | 0.941 | 34.270 | 1.00 | 55.85 | O |
| ATOM | 1040 | N | ALA | A | 67 | 40.199 | −0.016 | 33.618 | 1.00 | 65.15 | N |
| ATOM | 1042 | CA | ALA | A | 67 | 40.325 | 0.675 | 32.347 | 1.00 | 62.25 | C |
| ATOM | 1044 | CB | ALA | A | 67 | 41.569 | 1.522 | 32.373 | 1.00 | 61.31 | C |
| ATOM | 1048 | C | ALA | A | 67 | 40.382 | −0.318 | 31.177 | 1.00 | 67.15 | C |
| ATOM | 1049 | O | ALA | A | 67 | 41.444 | −0.568 | 30.618 | 1.00 | 67.60 | O |
| ATOM | 1050 | N | MET | A | 68 | 39.234 | −0.852 | 30.773 | 1.00 | 71.86 | N |
| ATOM | 1052 | CA | MET | A | 68 | 39.170 | −1.795 | 29.652 | 1.00 | 73.68 | C |
| ATOM | 1054 | CB | MET | A | 68 | 37.825 | −2.529 | 29.664 | 1.00 | 74.91 | C |
| ATOM | 1057 | CG | MET | A | 68 | 37.455 | −3.149 | 31.013 | 1.00 | 82.01 | C |
| ATOM | 1060 | SD | MET | A | 68 | 35.876 | −4.094 | 31.083 | 1.00 | 103.71 | S |
| ATOM | 1061 | CE | MET | A | 68 | 34.626 | −2.811 | 30.751 | 1.00 | 99.06 | C |
| ATOM | 1065 | C | MET | A | 68 | 39.367 | −1.127 | 28.287 | 1.00 | 71.15 | C |
| ATOM | 1066 | O | MET | A | 68 | 38.517 | −0.352 | 27.840 | 1.00 | 77.07 | O |
| ATOM | 1067 | N | GLY | A | 69 | 40.477 | −1.432 | 27.616 | 1.00 | 63.66 | N |
| ATOM | 1069 | CA | GLY | A | 69 | 40.641 | −1.048 | 26.229 | 1.00 | 50.80 | C |
| ATOM | 1072 | C | GLY | A | 69 | 39.647 | −1.852 | 25.410 | 1.00 | 53.47 | C |
| ATOM | 1073 | O | GLY | A | 69 | 39.628 | −3.074 | 25.520 | 1.00 | 51.37 | O |
| ATOM | 1074 | N | VAL | A | 70 | 38.805 | −1.183 | 24.616 | 1.00 | 52.47 | N |
| ATOM | 1076 | CA | VAL | A | 70 | 37.801 | −1.865 | 23.798 | 1.00 | 47.38 | C |
| ATOM | 1078 | CB | VAL | A | 70 | 36.391 | −1.670 | 24.373 | 1.00 | 44.41 | C |
| ATOM | 1080 | CG1 | VAL | A | 70 | 35.383 | −2.450 | 23.547 | 1.00 | 62.00 | C |
| ATOM | 1084 | CG2 | VAL | A | 70 | 36.334 | −2.161 | 25.805 | 1.00 | 48.22 | C |
| ATOM | 1088 | C | VAL | A | 70 | 37.768 | −1.437 | 22.334 | 1.00 | 43.33 | C |
| ATOM | 1089 | O | VAL | A | 70 | 37.697 | −0.260 | 22.034 | 1.00 | 48.62 | O |
| ATOM | 1090 | N | ASN | A | 71 | 37.769 | −2.408 | 21.425 | 1.00 | 48.63 | N |
| ATOM | 1092 | CA | ASN | A | 71 | 37.491 | −2.194 | 19.993 | 1.00 | 47.90 | C |
| ATOM | 1094 | CB | ASN | A | 71 | 37.908 | −3.478 | 19.259 | 1.00 | 45.93 | C |
| ATOM | 1097 | CG | ASN | A | 71 | 38.024 | −3.296 | 17.765 | 1.00 | 56.92 | C |
| ATOM | 1098 | OD1 | ASN | A | 71 | 37.039 | −3.028 | 17.074 | 1.00 | 60.60 | O |
| ATOM | 1099 | ND2 | ASN | A | 71 | 39.250 | −3.410 | 17.255 | 1.00 | 60.73 | N |
| ATOM | 1102 | C | ASN | A | 71 | 35.982 | −1.967 | 19.733 | 1.00 | 49.00 | C |
| ATOM | 1103 | O | ASN | A | 71 | 35.187 | −2.903 | 19.897 | 1.00 | 41.61 | O |
| ATOM | 1104 | N | LEU | A | 72 | 35.585 | −0.752 | 19.341 | 1.00 | 46.79 | N |
| ATOM | 1106 | CA | LEU | A | 72 | 34.171 | −0.412 | 19.208 | 1.00 | 44.63 | C |
| ATOM | 1108 | CB | LEU | A | 72 | 33.930 | 1.100 | 19.244 | 1.00 | 47.28 | C |
| ATOM | 1111 | CG | LEU | A | 72 | 34.228 | 1.763 | 20.588 | 1.00 | 42.84 | C |
| ATOM | 1113 | CD1 | LEU | A | 72 | 33.833 | 3.232 | 20.605 | 1.00 | 49.44 | C |
| ATOM | 1117 | CD2 | LEU | A | 72 | 33.572 | 1.014 | 21.725 | 1.00 | 40.78 | C |
| ATOM | 1121 | C | LEU | A | 72 | 33.600 | −0.991 | 17.927 | 1.00 | 47.18 | C |
| ATOM | 1122 | O | LEU | A | 72 | 32.376 | −1.163 | 17.802 | 1.00 | 35.78 | O |
| ATOM | 1123 | N | THR | A | 73 | 34.476 | −1.305 | 16.976 | 1.00 | 46.47 | N |
| ATOM | 1125 | CA | THR | A | 73 | 34.016 | −2.017 | 15.788 | 1.00 | 47.26 | C |
| ATOM | 1127 | CB | THR | A | 73 | 35.094 | −2.168 | 14.671 | 1.00 | 54.91 | C |
| ATOM | 1129 | OG1 | THR | A | 73 | 35.936 | −1.004 | 14.586 | 1.00 | 63.40 | O |
| ATOM | 1131 | CG2 | THR | A | 73 | 34.404 | −2.203 | 13.289 | 1.00 | 49.38 | C |
| ATOM | 1135 | C | THR | A | 73 | 33.468 | −3.379 | 16.182 | 1.00 | 40.48 | C |
| ATOM | 1136 | O | THR | A | 73 | 32.443 | −3.814 | 15.656 | 1.00 | 38.66 | O |
| ATOM | 1137 | N | SER | A | 74 | 34.166 | −4.034 | 17.102 | 1.00 | 38.55 | N |
| ATOM | 1139 | CA | SER | A | 74 | 33.874 | −5.400 | 17.486 | 1.00 | 38.90 | C |
| ATOM | 1141 | CB | SER | A | 74 | 35.081 | −6.063 | 18.157 | 1.00 | 37.35 | C |
| ATOM | 1144 | OG | SER | A | 74 | 36.257 | −5.874 | 17.382 | 1.00 | 60.30 | O |
| ATOM | 1146 | C | SER | A | 74 | 32.709 | −5.431 | 18.443 | 1.00 | 40.07 | C |
| ATOM | 1147 | O | SER | A | 74 | 31.907 | −6.344 | 18.387 | 1.00 | 42.17 | O |
| ATOM | 1148 | N | MET | A | 75 | 32.637 | −4.451 | 19.334 | 1.00 | 36.31 | N |
| ATOM | 1150 | CA | MET | A | 75 | 31.517 | −4.358 | 20.250 | 1.00 | 33.70 | C |
| ATOM | 1152 | CB | MET | A | 75 | 31.837 | −3.338 | 21.350 | 1.00 | 28.73 | C |
| ATOM | 1155 | CG | MET | A | 75 | 30.734 | −3.192 | 22.381 | 1.00 | 39.50 | C |
| ATOM | 1158 | SD | MET | A | 75 | 31.237 | −2.186 | 23.769 | 1.00 | 38.32 | S |
| ATOM | 1159 | CE | MET | A | 75 | 29.708 | −2.346 | 24.759 | 1.00 | 45.11 | C |
| ATOM | 1163 | C | MET | A | 75 | 30.194 | −4.013 | 19.550 | 1.00 | 36.34 | C |
| ATOM | 1164 | O | MET | A | 75 | 29.119 | −4.524 | 19.930 | 1.00 | 26.31 | O |
| ATOM | 1165 | N | SER | A | 76 | 30.252 | −3.129 | 18.558 | 1.00 | 36.77 | N |
| ATOM | 1167 | CA | SER | A | 76 | 29.070 | −2.849 | 17.720 | 1.00 | 33.51 | C |
| ATOM | 1169 | CB | SER | A | 76 | 29.310 | −1.680 | 16.753 | 1.00 | 42.41 | C |
| ATOM | 1172 | OG | SER | A | 76 | 30.465 | −1.870 | 15.954 | 1.00 | 54.11 | O |
| ATOM | 1174 | C | SER | A | 76 | 28.640 | −4.040 | 16.910 | 1.00 | 35.04 | C |
| ATOM | 1175 | O | SER | A | 76 | 27.452 | −4.255 | 16.756 | 1.00 | 42.66 | O |
| ATOM | 1176 | N | LYS | A | 77 | 29.584 | −4.799 | 16.347 | 1.00 | 33.12 | N |
| ATOM | 1178 | CA | LYS | A | 77 | 29.228 | −6.090 | 15.763 | 1.00 | 38.31 | C |
| ATOM | 1180 | CB | LYS | A | 77 | 30.492 | −6.810 | 15.290 | 1.00 | 45.55 | C |
| ATOM | 1183 | CG | LYS | A | 77 | 31.097 | −6.213 | 14.001 | 1.00 | 50.11 | C |
| ATOM | 1186 | CD | LYS | A | 77 | 32.013 | −7.193 | 13.267 | 1.00 | 56.09 | C |
| ATOM | 1189 | CE | LYS | A | 77 | 32.379 | −6.687 | 11.873 | 1.00 | 58.13 | C |
| ATOM | 1192 | NZ | LYS | A | 77 | 33.643 | −5.884 | 11.904 | 1.00 | 59.42 | N |
| ATOM | 1196 | C | LYS | A | 77 | 28.416 | −7.013 | 16.702 | 1.00 | 39.65 | C |
| ATOM | 1197 | O | LYS | A | 77 | 27.420 | −7.656 | 16.299 | 1.00 | 36.37 | O |
| ATOM | 1198 | N | ILE | A | 78 | 28.880 | −7.140 | 17.944 | 1.00 | 34.08 | N |
| ATOM | 1200 | CA | ILE | A | 78 | 28.302 | −8.114 | 18.868 | 1.00 | 34.21 | C |
| ATOM | 1202 | CB | ILE | A | 78 | 29.200 | −8.258 | 20.099 | 1.00 | 39.77 | C |
| ATOM | 1204 | CG1 | ILE | A | 78 | 30.353 | −9.225 | 19.809 | 1.00 | 34.63 | C |
| ATOM | 1207 | CD1 | ILE | A | 78 | 31.536 | −8.985 | 20.677 | 1.00 | 39.14 | C |
| ATOM | 1211 | CG2 | ILE | A | 78 | 28.423 | −8.714 | 21.325 | 1.00 | 37.43 | C |
| ATOM | 1215 | C | ILE | A | 78 | 26.900 | −7.605 | 19.242 | 1.00 | 32.20 | C |
| ATOM | 1216 | O | ILE | A | 78 | 26.944 | −8.371 | 19.348 | 1.00 | 43.21 | O |
| ATOM | 1217 | N | LEU | A | 79 | 26.767 | −6.293 | 19.350 | 1.00 | 29.58 | N |
| ATOM | 1219 | CA | LEU | A | 79 | 25.528 | −5.675 | 19.760 | 1.00 | 34.86 | C |
| ATOM | 1221 | CB | LEU | A | 79 | 26.767 | −4.221 | 20.131 | 1.00 | 38.47 | C |
| ATOM | 1224 | CG | LEU | A | 79 | 25.973 | −4.143 | 21.655 | 1.00 | 50.15 | C |
| ATOM | 1226 | CD1 | LEU | A | 79 | 26.966 | −3.069 | 22.045 | 1.00 | 59.25 | C |
| ATOM | 1230 | CD2 | LEU | A | 79 | 24.625 | −3.909 | 22.359 | 1.00 | 55.90 | C |
| ATOM | 1234 | C | LEU | A | 79 | 24.504 | −5.785 | 18.644 | 1.00 | 45.20 | C |
| ATOM | 1235 | O | LEU | A | 79 | 23.281 | −5.737 | 18.894 | 1.00 | 38.88 | O |
| ATOM | 1236 | N | LYS | A | 80 | 25.014 | −5.950 | 17.422 | 1.00 | 38.23 | N |
| ATOM | 1238 | CA | LYS | A | 80 | 24.142 | −6.191 | 16.274 | 1.00 | 46.87 | C |
| ATOM | 1240 | CB | LYS | A | 80 | 24.986 | −6.309 | 14.997 | 1.00 | 49.28 | C |
| ATOM | 1243 | CG | LYS | A | 80 | 24.554 | −5.356 | 13.886 | 1.00 | 49.44 | C |
| ATOM | 1246 | CD | LYS | A | 80 | 24.702 | −3.898 | 14.289 | 1.00 | 61.13 | C |
| ATOM | 1249 | CE | LYS | A | 80 | 24.446 | −2.959 | 13.117 | 1.00 | 72.87 | C |
| ATOM | 1252 | NZ | LYS | A | 80 | 25.552 | −2.989 | 12.112 | 1.00 | 81.83 | N |
| ATOM | 1256 | C | LYS | A | 80 | 23.335 | −7.480 | 16.486 | 1.00 | 44.49 | C |
| ATOM | 1257 | O | LYS | A | 80 | 22.139 | −7.545 | 16.207 | 1.00 | 39.79 | O |
| ATOM | 1258 | N | CYS | A | 81 | 24.042 | −8.482 | 16.999 | 1.00 | 39.91 | N |
| ATOM | 1260 | CA | CYS | A | 81 | 23.523 | −9.784 | 17.367 | 1.00 | 33.63 | C |
| ATOM | 1262 | CB | CYS | A | 81 | 24.698 | −10.672 | 17.731 | 1.00 | 29.25 | C |
| ATOM | 1265 | SG | CYS | A | 81 | 25.948 | −10.805 | 16.400 | 1.00 | 38.19 | S |
| ATOM | 1266 | C | CYS | A | 81 | 22.502 | −9.763 | 18.521 | 1.00 | 36.71 | C |
| ATOM | 1267 | O | CYS | A | 81 | 22.022 | −10.817 | 18.927 | 1.00 | 38.58 | O |
| ATOM | 1268 | N | ALA | A | 82 | 22.170 | −8.596 | 19.060 | 1.00 | 39.42 | N |
| ATOM | 1270 | CA | ALA | A | 82 | 21.227 | −8.559 | 20.187 | 1.00 | 41.36 | C |
| ATOM | 1272 | CB | ALA | A | 82 | 21.598 | −7.506 | 21.193 | 1.00 | 42.42 | C |
| ATOM | 1276 | C | ALA | A | 82 | 19.849 | −8.262 | 19.675 | 1.00 | 36.63 | C |
| ATOM | 1277 | O | ALA | A | 82 | 19.690 | −7.448 | 18.757 | 1.00 | 36.92 | O |
| ATOM | 1278 | N | GLY | A | 83 | 18.853 | −8.874 | 20.303 | 1.00 | 39.02 | N |
| ATOM | 1280 | CA | GLY | A | 83 | 17.473 | −8.549 | 19.979 | 1.00 | 37.42 | C |
| ATOM | 1283 | C | GLY | A | 83 | 17.112 | −7.146 | 20.424 | 1.00 | 31.30 | C |
| ATOM | 1284 | O | GLY | A | 83 | 17.721 | −6.600 | 21.326 | 1.00 | 36.54 | O |
| ATOM | 1285 | N | ASN | A | 84 | 16.094 | −6.564 | 19.805 | 1.00 | 42.20 | N |
| ATOM | 1287 | CA | ASN | A | 84 | 15.560 | −5.267 | 20.229 | 1.00 | 43.12 | C |
| ATOM | 1289 | CB | ASN | A | 84 | 14.412 | −4.881 | 19.305 | 1.00 | 47.21 | C |
| ATOM | 1292 | CG | ASN | A | 84 | 14.844 | −3.942 | 18.213 | 1.00 | 53.90 | C |
| ATOM | 1293 | OD1 | ASN | A | 84 | 15.786 | −3.162 | 18.395 | 1.00 | 50.68 | O |
| ATOM | 1294 | ND2 | ASN | A | 84 | 14.170 | −4.019 | 17.059 | 1.00 | 46.23 | N |
| ATOM | 1297 | C | ASN | A | 84 | 15.029 | −5.221 | 21.669 | 1.00 | 41.93 | C |
| ATOM | 1298 | O | ASN | A | 84 | 15.016 | −4.174 | 22.330 | 1.00 | 47.34 | O |
| ATOM | 1299 | N | GLU | A | 85 | 14.496 | −6.342 | 22.119 | 1.00 | 41.13 | N |
| ATOM | 1301 | CA | GLU | A | 85 | 13.931 | −6.410 | 23.452 | 1.00 | 45.32 | C |
| ATOM | 1303 | CB | GLU | A | 85 | 12.421 | −6.652 | 23.356 | 1.00 | 52.09 | C |
| ATOM | 1306 | CG | GLU | A | 85 | 11.698 | −5.422 | 22.824 | 1.00 | 51.63 | C |
| ATOM | 1309 | CD | GLU | A | 85 | 10.317 | −5.200 | 23.425 | 1.00 | 76.85 | C |
| ATOM | 1310 | OE1 | GLU | A | 85 | 10.068 | −4.070 | 23.922 | 1.00 | 71.39 | O |
| ATOM | 1311 | OE2 | GLU | A | 85 | 9.484 | −6.141 | 23.380 | 1.00 | 76.49 | O |
| ATOM | 1312 | C | GLU | A | 85 | 14.617 | −7.424 | 24.361 | 1.00 | 41.03 | C |
| ATOM | 1313 | O | GLU | A | 85 | 14.096 | −7.724 | 25.419 | 1.00 | 51.35 | O |
| ATOM | 1314 | N | ASP | A | 86 | 15.795 | −7.910 | 23.968 | 1.00 | 45.82 | N |
| ATOM | 1316 | CA | ASP | A | 86 | 16.674 | −8.697 | 24.841 | 1.00 | 46.58 | C |
| ATOM | 1318 | CB | ASP | A | 86 | 17.939 | −9.108 | 24.057 | 1.00 | 52.72 | C |
| ATOM | 1321 | CG | ASP | A | 86 | 17.688 | −10.205 | 23.018 | 1.00 | 62.65 | C |
| ATOM | 1322 | OD1 | ASP | A | 86 | 16.504 | −10.509 | 22.718 | 1.00 | 70.05 | O |
| ATOM | 1323 | OD2 | ASP | A | 86 | 18.634 | −10.804 | 22.440 | 1.00 | 57.86 | O |
| ATOM | 1324 | C | ASP | A | 86 | 17.126 | −7.937 | 26.109 | 1.00 | 40.33 | C |
| ATOM | 1325 | O | ASP | A | 86 | 17.411 | −6.741 | 26.074 | 1.00 | 38.15 | O |
| ATOM | 1326 | N | ILE | A | 87 | 17.255 | −8.668 | 27.208 | 1.00 | 35.31 | N |
| ATOM | 1328 | CA | ILE | A | 87 | 18.024 | −8.235 | 28.358 | 1.00 | 37.01 | C |
| ATOM | 1330 | CB | ILE | A | 87 | 17.716 | −9.128 | 29.638 | 1.00 | 35.00 | C |
| ATOM | 1332 | CG1 | ILE | A | 87 | 16.248 | −9.090 | 30.107 | 1.00 | 30.47 | C |
| ATOM | 1335 | CD1 | ILE | A | 87 | 15.349 | −8.382 | 29.184 | 1.00 | 47.63 | C |
| ATOM | 1339 | CG2 | ILE | A | 87 | 18.556 | −8.705 | 30.821 | 1.00 | 32.32 | C |
| ATOM | 1343 | C | ILE | A | 87 | 19.498 | −8.395 | 27.986 | 1.00 | 41.94 | C |
| ATOM | 1344 | O | ILE | A | 87 | 19.955 | −9.513 | 27.663 | 1.00 | 43.75 | O |
| ATOM | 1345 | N | ILE | A | 88 | 20.246 | −7.303 | 28.149 | 1.00 | 45.50 | N |
| ATOM | 1347 | CA | ILE | A | 88 | 21.691 | −7.280 | 27.909 | 1.00 | 41.45 | C |
| ATOM | 1349 | CB | ILE | A | 88 | 22.050 | −6.097 | 27.023 | 1.00 | 43.36 | C |
| ATOM | 1351 | CG1 | ILE | A | 88 | 21.253 | −6.159 | 25.724 | 1.00 | 40.96 | C |
| ATOM | 1354 | CD1 | ILE | A | 88 | 21.792 | −5.211 | 24.663 | 1.00 | 38.63 | C |
| ATOM | 1358 | CG2 | ILE | A | 88 | 23.548 | −6.080 | 26.689 | 1.00 | 34.92 | C |
| ATOM | 1362 | C | ILE | A | 88 | 22.421 | −7.143 | 29.224 | 1.00 | 43.88 | C |
| ATOM | 1363 | O | ILE | A | 88 | 22.177 | −6.204 | 29.998 | 1.00 | 47.59 | O |
| ATOM | 1364 | N | THR | A | 89 | 23.325 | −8.084 | 29.459 | 1.00 | 36.79 | N |
| ATOM | 1366 | CA | THR | A | 89 | 24.186 | −8.058 | 30.637 | 1.00 | 37.86 | C |
| ATOM | 1368 | CB | THR | A | 89 | 23.967 | −9.317 | 31.494 | 1.00 | 35.63 | C |
| ATOM | 1370 | OG1 | THR | A | 89 | 22.572 | −9.430 | 31.849 | 1.00 | 43.04 | O |
| ATOM | 1372 | CG2 | THR | A | 89 | 24.715 | −9.160 | 32.844 | 1.00 | 35.39 | C |
| ATOM | 1376 | C | THR | A | 89 | 25.656 | −7.987 | 30.271 | 1.00 | 32.39 | C |
| ATOM | 1377 | O | THR | A | 89 | 26.157 | −8.876 | 29.634 | 1.00 | 42.49 | O |
| ATOM | 1378 | N | LEU | A | 90 | 26.366 | −6.990 | 30.771 | 1.00 | 40.08 | N |
| ATOM | 1380 | CA | LEU | A | 90 | 27.792 | −6.854 | 30.542 | 1.00 | 35.25 | C |
| ATOM | 1382 | CB | LEU | A | 90 | 28.200 | −5.385 | 30.388 | 1.00 | 40.77 | C |
| ATOM | 1385 | CG | LEU | A | 90 | 27.432 | −4.519 | 29.386 | 1.00 | 32.38 | C |
| ATOM | 1387 | CD1 | LEU | A | 90 | 28.027 | −3.131 | 29.336 | 1.00 | 48.92 | C |
| ATOM | 1391 | CD2 | LEU | A | 90 | 27.508 | −5.139 | 28.023 | 1.00 | 36.82 | C |
| ATOM | 1395 | C | LEU | A | 90 | 28.528 | −7.417 | 31.726 | 1.00 | 38.90 | C |
| ATOM | 1396 | O | LEU | A | 90 | 28.140 | −7.157 | 32.854 | 1.00 | 43.47 | O |
| ATOM | 1397 | N | ARG | A | 91 | 29.668 | −8.060 | 31.472 | 1.00 | 44.03 | N |
| ATOM | 1399 | CA | ARG | A | 91 | 30.368 | −8.808 | 32.510 | 1.00 | 46.26 | C |
| ATOM | 1401 | CB | ARG | A | 91 | 29.754 | −10.197 | 32.663 | 1.00 | 51.02 | C |
| ATOM | 1404 | CG | ARG | A | 91 | 29.947 | −10.851 | 34.028 | 1.00 | 70.30 | C |
| ATOM | 1407 | CD | ARG | A | 91 | 30.081 | −12.399 | 34.016 | 1.00 | 80.13 | C |
| ATOM | 1410 | NE | ARG | A | 91 | 29.078 | −13.141 | 33.237 | 1.00 | 74.86 | N |
| ATOM | 1412 | CZ | ARG | A | 91 | 27.812 | −13.358 | 33.605 | 1.00 | 93.26 | C |
| ATOM | 1413 | NH1 | ARG | A | 91 | 27.326 | −12.822 | 34.719 | 1.00 | 98.67 | N |
| ATOM | 1416 | NH2 | ARG | A | 91 | 27.004 | −14.085 | 32.839 | 1.00 | 83.54 | N |
| ATOM | 1419 | C | ARG | A | 91 | 31.861 | −8.952 | 32.278 | 1.00 | 46.74 | C |
| ATOM | 1420 | O | ARG | A | 91 | 32.343 | −9.257 | 31.188 | 1.00 | 48.12 | O |
| ATOM | 1421 | N | ALA | A | 92 | 32.614 | −8.749 | 33.344 | 1.00 | 49.80 | N |
| ATOM | 1423 | CA | ALA | A | 92 | 34.023 | −9.071 | 33.299 | 1.00 | 49.42 | C |
| ATOM | 1425 | CB | ALA | A | 92 | 34.800 | −7.794 | 33.018 | 1.00 | 31.55 | C |
| ATOM | 1429 | C | ALA | A | 92 | 34.447 | −9.723 | 34.617 | 1.00 | 51.98 | C |
| ATOM | 1430 | O | ALA | A | 92 | 34.350 | −9.102 | 35.673 | 1.00 | 55.73 | O |
| ATOM | 1431 | N | GLU | A | 93 | 34.896 | −10.971 | 34.576 | 1.00 | 65.71 | N |
| ATOM | 1433 | CA | GLU | A | 93 | 35.752 | −11.476 | 35.651 | 1.00 | 77.26 | C |
| ATOM | 1435 | CB | GLU | A | 93 | 36.005 | −12.980 | 35.511 | 1.00 | 80.26 | C |
| ATOM | 1438 | CG | GLU | A | 93 | 35.006 | −13.849 | 36.262 | 1.00 | 94.06 | C |
| ATOM | 1441 | CD | GLU | A | 93 | 35.445 | −14.203 | 37.681 | 1.00 | 106.88 | C |
| ATOM | 1442 | OE1 | GLU | A | 93 | 36.207 | −15.190 | 37.828 | 1.00 | 113.76 | O |
| ATOM | 1443 | OE2 | GLU | A | 93 | 35.005 | −13.524 | 38.648 | 1.00 | 80.69 | O |
| ATOM | 1444 | C | GLU | A | 93 | 37.078 | −10.699 | 35.652 | 1.00 | 83.55 | C |
| ATOM | 1445 | O | GLU | A | 93 | 37.303 | −9.825 | 34.811 | 1.00 | 82.37 | O |
| ATOM | 1446 | N | ASP | A | 94 | 37.962 | −11.028 | 36.588 | 1.00 | 93.15 | N |
| ATOM | 1448 | CA | ASP | A | 94 | 38.994 | −10.091 | 37.037 | 1.00 | 97.00 | C |
| ATOM | 1450 | CB | ASP | A | 94 | 39.252 | −10.305 | 38.527 | 1.00 | 97.83 | C |
| ATOM | 1453 | CG | ASP | A | 94 | 38.142 | −9.748 | 39.396 | 1.00 | 103.48 | C |
| ATOM | 1454 | OD1 | ASP | A | 94 | 37.155 | −10.482 | 39.647 | 1.00 | 108.15 | O |
| ATOM | 1455 | OD2 | ASP | A | 94 | 38.186 | −8.588 | 39.871 | 1.00 | 106.70 | O |
| ATOM | 1456 | C | ASP | A | 94 | 40.322 | −10.181 | 36.277 | 1.00 | 99.46 | C |
| ATOM | 1457 | O | ASP | A | 94 | 40.647 | −9.318 | 35.456 | 1.00 | 100.21 | O |
| ATOM | 1458 | N | ASN | A | 95 | 41.108 | −11.207 | 36.583 | 1.00 | 100.57 | N |
| ATOM | 1460 | CA | ASN | A | 95 | 42.419 | −11.372 | 35.959 | 1.00 | 102.06 | C |
| ATOM | 1462 | CB | ASN | A | 95 | 43.328 | −12.229 | 36.852 | 1.00 | 99.63 | C |
| ATOM | 1465 | CG | ASN | A | 95 | 43.497 | −11.642 | 38.247 | 1.00 | 98.02 | C |
| ATOM | 1466 | OD1 | ASN | A | 95 | 42.588 | −11.703 | 39.074 | 1.00 | 84.01 | O |
| ATOM | 1467 | ND2 | ASN | A | 95 | 44.660 | −11.056 | 38.507 | 1.00 | 98.10 | N |
| ATOM | 1470 | C | ASN | A | 95 | 42.295 | −11.953 | 34.540 | 1.00 | 100.91 | C |
| ATOM | 1471 | O | ASN | A | 95 | 43.290 | −12.089 | 33.820 | 1.00 | 102.61 | O |
| ATOM | 1472 | N | ALA | A | 96 | 41.063 | −12.293 | 34.157 | 1.00 | 97.06 | N |
| ATOM | 1474 | CA | ALA | A | 96 | 40.648 | −12.350 | 32.754 | 1.00 | 93.52 | C |
| ATOM | 1476 | CB | ALA | A | 96 | 39.310 | −13.111 | 32.615 | 1.00 | 92.44 | C |
| ATOM | 1480 | C | ALA | A | 96 | 40.504 | −10.928 | 32.200 | 1.00 | 87.93 | C |
| ATOM | 1481 | O | ALA | A | 96 | 39.868 | −10.068 | 32.819 | 1.00 | 87.21 | O |
| ATOM | 1482 | N | ASP | A | 97 | 41.132 | −10.670 | 31.057 | 1.00 | 78.94 | N |
| ATOM | 1484 | CA | ASP | A | 97 | 40.832 | −9.468 | 30.296 | 1.00 | 75.81 | C |
| ATOM | 1486 | CB | ASP | A | 97 | 42.100 | −8.865 | 29.672 | 1.00 | 80.13 | C |
| ATOM | 1489 | CG | ASP | A | 97 | 43.154 | −8.479 | 30.701 | 1.00 | 89.98 | C |
| ATOM | 1490 | OD1 | ASP | A | 97 | 42.975 | −7.449 | 31.388 | 1.00 | 107.25 | O |
| ATOM | 1491 | OD2 | ASP | A | 97 | 44.223 | −9.110 | 30.850 | 1.00 | 93.13 | O |
| ATOM | 1492 | C | ASP | A | 97 | 39.871 | −9.875 | 29.176 | 1.00 | 66.75 | C |
| ATOM | 1493 | O | ASP | A | 97 | 40.298 | −10.002 | 28.037 | 1.00 | 53.17 | O |
| ATOM | 1494 | N | THR | A | 98 | 38.619 | −10.167 | 29.522 | 1.00 | 52.23 | N |
| ATOM | 1496 | CA | THR | A | 98 | 37.562 | −10.327 | 28.535 | 1.00 | 59.33 | C |
| ATOM | 1498 | CB | THR | A | 98 | 37.241 | −11.825 | 28.296 | 1.00 | 62.68 | C |
| ATOM | 1500 | OG1 | THR | A | 98 | 37.338 | −12.560 | 29.525 | 1.00 | 75.29 | O |
| ATOM | 1502 | CG2 | THR | A | 98 | 38.285 | −12.476 | 27.392 | 1.00 | 67.80 | C |
| ATOM | 1506 | C | THR | A | 98 | 36.309 | −9.598 | 29.011 | 1.00 | 51.24 | C |
| ATOM | 1507 | O | THR | A | 98 | 35.992 | −9.602 | 30.202 | 1.00 | 48.94 | O |
| ATOM | 1508 | N | LEU | A | 99 | 35.651 | −8.894 | 28.097 | 1.00 | 46.88 | N |
| ATOM | 1510 | CA | LEU | A | 99 | 34.287 | −8.463 | 28.329 | 1.00 | 35.13 | C |
| ATOM | 1512 | CB | LEU | A | 99 | 34.050 | −7.093 | 27.716 | 1.00 | 43.34 | C |
| ATOM | 1515 | CG | LEU | A | 99 | 32.718 | −6.408 | 28.027 | 1.00 | 34.74 | C |
| ATOM | 1517 | CD1 | LEU | A | 99 | 32.777 | −5.876 | 29.441 | 1.00 | 40.71 | C |
| ATOM | 1521 | CD2 | LEU | A | 99 | 32.445 | −5.263 | 27.083 | 1.00 | 45.38 | C |
| ATOM | 1525 | C | LEU | A | 99 | 33.348 | −9.486 | 27.720 | 1.00 | 33.52 | C |
| ATOM | 1526 | O | LEU | A | 99 | 33.397 | −9.745 | 26.535 | 1.00 | 38.19 | O |
| ATOM | 1527 | N | ALA | A | 100 | 32.489 | −10.059 | 28.554 | 1.00 | 38.29 | N |
| ATOM | 1529 | CA | ALA | A | 100 | 31.357 | −10.864 | 28.112 | 1.00 | 33.99 | C |
| ATOM | 1531 | CB | ALA | A | 100 | 31.089 | −11.970 | 29.100 | 1.00 | 33.27 | C |
| ATOM | 1535 | C | ALA | A | 100 | 30.123 | −10.006 | 27.932 | 1.00 | 34.57 | C |
| ATOM | 1536 | O | ALA | A | 100 | 29.960 | −8.984 | 28.614 | 1.00 | 41.79 | O |
| ATOM | 1537 | N | LEU | A | 101 | 29.286 | −10.396 | 26.971 | 1.00 | 41.14 | N |
| ATOM | 1539 | CA | LEU | A | 101 | 27.972 | −9.782 | 26.772 | 1.00 | 32.76 | C |
| ATOM | 1541 | CB | LEU | A | 101 | 27.956 | −8.838 | 25.602 | 1.00 | 25.48 | C |
| ATOM | 1544 | CG | LEU | A | 101 | 29.027 | −7.754 | 25.613 | 1.00 | 35.57 | C |
| ATOM | 1546 | CD1 | LEU | A | 101 | 30.322 | −8.348 | 25.054 | 1.00 | 42.88 | C |
| ATOM | 1550 | CD2 | LEU | A | 101 | 28.572 | −6.610 | 24.731 | 1.00 | 47.25 | C |
| ATOM | 1554 | C | LEU | A | 101 | 26.896 | −10.814 | 26.539 | 1.00 | 40.03 | C |
| ATOM | 1555 | O | LEU | A | 101 | 27.007 | −11.598 | 25.610 | 1.00 | 48.49 | O |
| ATOM | 1556 | N | VAL | A | 102 | 25.896 | −10.831 | 27.421 | 1.00 | 35.95 | N |
| ATOM | 1558 | CA | VAL | A | 102 | 24.871 | −11.877 | 27.434 | 1.00 | 43.43 | C |
| ATOM | 1560 | CB | VAL | A | 102 | 24.812 | −12.577 | 28.818 | 1.00 | 43.84 | C |
| ATOM | 1562 | CG1 | VAL | A | 102 | 23.855 | −13.780 | 28.833 | 1.00 | 36.73 | C |
| ATOM | 1566 | CG2 | VAL | A | 102 | 26.221 | −13.045 | 29.180 | 1.00 | 42.91 | C |
| ATOM | 1570 | C | VAL | A | 102 | 23.535 | −11.238 | 27.109 | 1.00 | 37.97 | C |
| ATOM | 1571 | O | VAL | A | 102 | 23.170 | −10.239 | 27.734 | 1.00 | 37.03 | O |
| ATOM | 1572 | N | PHE | A | 103 | 22.880 | −11.809 | 26.098 | 1.00 | 31.95 | N |
| ATOM | 1574 | CA | PHE | A | 103 | 21.588 | −11.418 | 25.554 | 1.00 | 34.77 | C |
| ATOM | 1576 | CB | PHE | A | 103 | 21.696 | −11.289 | 24.024 | 1.00 | 29.03 | C |
| ATOM | 1579 | CG | PHE | A | 103 | 22.816 | −10.405 | 23.559 | 1.00 | 31.38 | C |
| ATOM | 1580 | CD1 | PHE | A | 103 | 23.228 | −9.319 | 24.317 | 1.00 | 38.24 | C |
| ATOM | 1582 | CE1 | PHE | A | 103 | 24.222 | −8.503 | 23.868 | 1.00 | 36.53 | C |
| ATOM | 1584 | CZ | PHE | A | 103 | 24.819 | −8.732 | 22.644 | 1.00 | 34.16 | C |
| ATOM | 1586 | CE2 | PHE | A | 103 | 24.420 | −9.790 | 21.875 | 1.00 | 32.09 | C |
| ATOM | 1588 | CD2 | PHE | A | 103 | 23.426 | −10.621 | 22.330 | 1.00 | 39.34 | C |
| ATOM | 1590 | C | PHE | A | 103 | 20.578 | −12.528 | 25.867 | 1.00 | 37.24 | C |
| ATOM | 1591 | O | PHE | A | 103 | 20.719 | −13.640 | 25.377 | 1.00 | 41.65 | O |
| ATOM | 1592 | N | GLU | A | 104 | 19.562 | −12.228 | 26.672 | 1.00 | 36.70 | N |
| ATOM | 1594 | CA | GLU | A | 104 | 18.529 | −13.203 | 27.008 | 1.00 | 39.37 | C |
| ATOM | 1596 | CB | GLU | A | 104 | 18.316 | −13.278 | 28.527 | 1.00 | 43.82 | C |
| ATOM | 1599 | CG | GLU | A | 104 | 19.563 | −13.633 | 29.339 | 1.00 | 47.13 | C |
| ATOM | 1602 | CD | GLU | A | 104 | 19.317 | −13.673 | 30.842 | 1.00 | 64.97 | C |
| ATOM | 1603 | OE1 | GLU | A | 104 | 19.020 | −12.608 | 31.440 | 1.00 | 58.38 | O |
| ATOM | 1604 | OE2 | GLU | A | 104 | 19.422 | −14.777 | 31.424 | 1.00 | 59.08 | O |
| ATOM | 1605 | C | GLU | A | 104 | 17.267 | −12.734 | 26.314 | 1.00 | 45.75 | C |
| ATOM | 1606 | O | GLU | A | 104 | 16.781 | −11.627 | 26.576 | 1.00 | 42.68 | O |
| ATOM | 1607 | N | ALA | A | 105 | 16.833 | −13.525 | 25.340 | 1.00 | 53.98 | N |
| ATOM | 1609 | CA | ALA | A | 105 | 15.638 | −13.225 | 24.564 | 1.00 | 66.49 | C |
| ATOM | 1611 | CB | ALA | A | 105 | 15.423 | −14.298 | 23.493 | 1.00 | 63.54 | C |
| ATOM | 1615 | C | ALA | A | 105 | 14.416 | −13.097 | 25.488 | 1.00 | 70.93 | C |
| ATOM | 1616 | O | ALA | A | 105 | 14.402 | −13.665 | 26.580 | 1.00 | 70.59 | O |
| ATOM | 1617 | N | PRO | A | 106 | 13.435 | −12.291 | 25.080 | 1.00 | 79.51 | N |
| ATOM | 1618 | CA | PRO | A | 106 | 12.286 | −11.939 | 25.934 | 1.00 | 85.40 | C |
| ATOM | 1620 | CB | PRO | A | 106 | 11.306 | −11.246 | 24.968 | 1.00 | 82.69 | C |
| ATOM | 1623 | CG | PRO | A | 106 | 11.927 | −11.309 | 23.598 | 1.00 | 79.86 | C |
| ATOM | 1626 | CD | PRO | A | 106 | 13.392 | −11.589 | 23.785 | 1.00 | 77.65 | C |
| ATOM | 1629 | C | PRO | A | 106 | 11.581 | −13.082 | 26.673 | 1.00 | 91.04 | C |
| ATOM | 1630 | O | PRO | A | 106 | 11.194 | −12.890 | 27.828 | 1.00 | 94.66 | O |
| ATOM | 1631 | N | ASN | A | 107 | 11.389 | −14.231 | 26.031 | 1.00 | 97.35 | N |
| ATOM | 1633 | CA | ASN | A | 107 | 10.668 | −15.345 | 26.653 | 1.00 | 102.44 | C |
| ATOM | 1635 | CB | ASN | A | 107 | 9.431 | −15.692 | 25.807 | 1.00 | 103.48 | C |
| ATOM | 1638 | CG | ASN | A | 107 | 8.763 | −14.452 | 25.209 | 1.00 | 102.01 | C |
| ATOM | 1639 | OD1 | ASN | A | 107 | 9.035 | −13.323 | 25.620 | 1.00 | 89.49 | O |
| ATOM | 1640 | ND2 | ASN | A | 107 | 7.885 | −14.663 | 24.236 | 1.00 | 92.53 | N |
| ATOM | 1643 | C | ASN | A | 107 | 11.557 | −16.587 | 26.858 | 1.00 | 105.62 | C |
| ATOM | 1644 | O | ASN | A | 107 | 11.454 | −17.554 | 26.093 | 1.00 | 106.40 | O |
| ATOM | 1645 | N | GLN | A | 108 | 12.350 | −16.576 | 27.939 | 1.00 | 104.75 | N |
| ATOM | 1647 | CA | GLN | A | 108 | 13.663 | −17.253 | 28.029 | 1.00 | 104.54 | C |
| ATOM | 1649 | CB | GLN | A | 108 | 14.019 | −17.569 | 29.490 | 1.00 | 103.94 | C |
| ATOM | 1652 | CG | GLN | A | 108 | 13.696 | −16.446 | 30.512 | 1.00 | 104.87 | C |
| ATOM | 1655 | CD | GLN | A | 108 | 13.609 | −17.008 | 31.925 | 1.00 | 106.11 | C |
| ATOM | 1656 | OE1 | GLN | A | 108 | 12.878 | −17.973 | 32.173 | 1.00 | 109.54 | O |
| ATOM | 1657 | NE2 | GLN | A | 108 | 14.362 | −16.420 | 32.849 | 1.00 | 96.38 | N |
| ATOM | 1660 | C | GLN | A | 108 | 13.950 | −18.498 | 27.153 | 1.00 | 101.47 | C |
| ATOM | 1661 | O | GLN | A | 108 | 14.312 | −19.564 | 27.662 | 1.00 | 101.63 | O |
| ATOM | 1662 | N | GLU | A | 109 | 13.913 | −18.323 | 25.835 | 1.00 | 95.83 | N |
| ATOM | 1664 | CA | GLU | A | 109 | 14.094 | −19.420 | 24.879 | 1.00 | 89.18 | C |
| ATOM | 1666 | CB | GLU | A | 109 | 13.446 | −19.013 | 23.542 | 1.00 | 94.20 | C |
| ATOM | 1669 | CG | GLU | A | 109 | 12.332 | −17.967 | 23.637 | 1.00 | 98.73 | C |
| ATOM | 1672 | CD | GLU | A | 109 | 12.557 | −16.734 | 22.765 | 1.00 | 108.63 | C |
| ATOM | 1673 | OE1 | GLU | A | 109 | 12.878 | −16.869 | 21.561 | 1.00 | 97.08 | O |
| ATOM | 1674 | OE2 | GLU | A | 109 | 12.385 | −15.608 | 23.281 | 1.00 | 116.71 | O |
| ATOM | 1675 | C | GLU | A | 109 | 15.583 | −19.737 | 24.638 | 1.00 | 76.24 | C |
| ATOM | 1676 | O | GLU | A | 109 | 15.998 | −20.882 | 24.456 | 1.00 | 70.84 | O |
| ATOM | 1677 | N | LYS | A | 110 | 16.384 | −18.687 | 24.711 | 1.00 | 65.35 | N |
| ATOM | 1679 | CA | LYS | A | 110 | 17.616 | −18.556 | 23.953 | 1.00 | 57.15 | C |
| ATOM | 1681 | CB | LYS | A | 110 | 17.278 | −18.043 | 22.536 | 1.00 | 50.43 | C |
| ATOM | 1684 | CG | LYS | A | 110 | 18.366 | −17.260 | 21.819 | 1.00 | 54.16 | C |
| ATOM | 1687 | CD | LYS | A | 110 | 18.023 | −17.107 | 20.334 | 1.00 | 54.78 | C |
| ATOM | 1690 | CE | LYS | A | 110 | 19.153 | −16.459 | 19.520 | 1.00 | 52.96 | C |
| ATOM | 1693 | NZ | LYS | A | 110 | 18.707 | −15.244 | 18.780 | 1.00 | 38.07 | N |
| ATOM | 1697 | C | LYS | A | 110 | 18.463 | −17.554 | 24.747 | 1.00 | 52.43 | C |
| ATOM | 1698 | O | LYS | A | 110 | 17.986 | −16.454 | 25.070 | 1.00 | 51.41 | O |
| ATOM | 1699 | N | VAL | A | 111 | 19.642 | −18.009 | 25.170 | 1.00 | 41.38 | N |
| ATOM | 1701 | CA | VAL | A | 111 | 20.728 | −17.157 | 25.642 | 1.00 | 43.95 | C |
| ATOM | 1703 | CB | VAL | A | 111 | 21.238 | −17.683 | 26.995 | 1.00 | 46.57 | C |
| ATOM | 1705 | CG1 | VAL | A | 111 | 22.233 | −16.733 | 27.630 | 1.00 | 44.27 | C |
| ATOM | 1709 | CG2 | VAL | A | 111 | 20.064 | −17.967 | 27.901 | 1.00 | 49.49 | C |
| ATOM | 1713 | C | VAL | A | 111 | 21.901 | −17.181 | 24.659 | 1.00 | 41.66 | C |
| ATOM | 1714 | O | VAL | A | 111 | 22.356 | −18.240 | 24.240 | 1.00 | 45.46 | O |
| ATOM | 1715 | N | SER | A | 112 | 22.413 | −15.997 | 24.353 | 1.00 | 47.43 | N |
| ATOM | 1717 | CA | SER | A | 112 | 23.604 | −15.780 | 23.524 | 1.00 | 41.08 | C |
| ATOM | 1719 | CB | SER | A | 112 | 23.273 | −14.694 | 22.481 | 1.00 | 35.34 | C |
| ATOM | 1722 | OG | SER | A | 112 | 22.223 | −15.073 | 21.616 | 1.00 | 31.48 | O |
| ATOM | 1724 | C | SER | A | 112 | 24.731 | −15.254 | 24.443 | 1.00 | 42.30 | C |
| ATOM | 1725 | O | SER | A | 112 | 24.549 | −14.218 | 25.063 | 1.00 | 49.19 | O |
| ATOM | 1726 | N | ASP | A | 113 | 25.861 | −15.960 | 24.537 | 1.00 | 42.90 | N |
| ATOM | 1728 | CA | ASP | A | 113 | 27.063 | −15.518 | 25.265 | 1.00 | 41.61 | C |
| ATOM | 1730 | CB | ASP | A | 113 | 27.597 | −16.693 | 26.105 | 1.00 | 47.24 | C |
| ATOM | 1733 | CG | ASP | A | 113 | 28.639 | −16.278 | 27.117 | 1.00 | 57.59 | C |
| ATOM | 1734 | OD1 | ASP | A | 113 | 29.197 | −15.168 | 26.977 | 1.00 | 73.10 | O |
| ATOM | 1735 | OD2 | ASP | A | 113 | 29.006 | −17.021 | 28.059 | 1.00 | 80.88 | O |
| ATOM | 1736 | C | ASP | A | 113 | 28.178 | −15.069 | 24.313 | 1.00 | 37.11 | C |
| ATOM | 1737 | O | ASP | A | 113 | 28.688 | −15.878 | 23.550 | 1.00 | 47.13 | O |
| ATOM | 1738 | N | TYR | A | 114 | 28.543 | −13.785 | 24.325 | 1.00 | 35.63 | N |
| ATOM | 1740 | CA | TYR | A | 114 | 29.661 | −13.270 | 23.521 | 1.00 | 37.13 | C |
| ATOM | 1742 | CB | TYR | A | 114 | 29.192 | −12.157 | 22.556 | 1.00 | 34.67 | C |
| ATOM | 1745 | CG | TYR | A | 114 | 28.165 | −12.578 | 21.512 | 1.00 | 35.19 | C |
| ATOM | 1746 | CD1 | TYR | A | 114 | 26.801 | −12.466 | 21.757 | 1.00 | 30.94 | C |
| ATOM | 1748 | CE1 | TYR | A | 114 | 25.854 | −12.909 | 20.823 | 1.00 | 27.36 | C |
| ATOM | 1750 | CZ | TYR | A | 114 | 26.253 | −13.449 | 19.630 | 1.00 | 34.47 | C |
| ATOM | 1751 | OH | TYR | A | 114 | 25.303 | −13.855 | 18.712 | 1.00 | 28.77 | O |
| ATOM | 1753 | CE2 | TYR | A | 114 | 27.611 | −13.598 | 19.365 | 1.00 | 39.60 | C |
| ATOM | 1755 | CD2 | TYR | A | 114 | 28.556 | −13.164 | 20.306 | 1.00 | 27.79 | C |
| ATOM | 1757 | C | TYR | A | 114 | 30.848 | −12.743 | 24.342 | 1.00 | 39.28 | C |
| ATOM | 1758 | O | TYR | A | 114 | 30.697 | −12.319 | 25.485 | 1.00 | 38.81 | O |
| ATOM | 1759 | N | GLU | A | 115 | 32.040 | −12.778 | 23.758 | 1.00 | 39.30 | N |
| ATOM | 1761 | CA | GLU | A | 115 | 33.243 | −12.372 | 24.471 | 1.00 | 36.01 | C |
| ATOM | 1763 | CB | GLU | A | 115 | 33.993 | −13.568 | 25.048 | 1.00 | 43.31 | C |
| ATOM | 1766 | CG | GLU | A | 115 | 33.170 | −14.494 | 25.908 | 1.00 | 56.21 | C |
| ATOM | 1769 | CD | GLU | A | 115 | 33.997 | −15.643 | 26.442 | 1.00 | 59.39 | C |
| ATOM | 1770 | OE1 | GLU | A | 115 | 33.628 | −16.820 | 26.198 | 1.00 | 85.79 | O |
| ATOM | 1771 | OE2 | GLU | A | 115 | 35.008 | −15.358 | 27.112 | 1.00 | 63.34 | O |
| ATOM | 1772 | C | GLU | A | 115 | 34.205 | −11.715 | 23.530 | 1.00 | 32.81 | C |
| ATOM | 1773 | O | GLU | A | 115 | 34.415 | −12.237 | 22.454 | 1.00 | 36.79 | O |
| ATOM | 1774 | N | MET | A | 116 | 34.819 | −10.612 | 23.957 | 1.00 | 33.24 | N |
| ATOM | 1776 | CA | MET | A | 116 | 35.760 | −9.900 | 23.122 | 1.00 | 41.76 | C |
| ATOM | 1778 | CB | MET | A | 116 | 35.088 | −8.647 | 22.577 | 1.00 | 41.54 | C |
| ATOM | 1781 | CG | MET | A | 116 | 34.505 | −7.769 | 23.597 | 1.00 | 46.82 | C |
| ATOM | 1784 | SD | MET | A | 116 | 33.760 | −6.224 | 22.893 | 1.00 | 54.92 | S |
| ATOM | 1785 | CE | MET | A | 116 | 35.234 | −5.447 | 22.245 | 1.00 | 54.08 | C |
| ATOM | 1789 | C | MET | A | 116 | 37.021 | −9.514 | 23.890 | 1.00 | 48.10 | C |
| ATOM | 1790 | O | MET | A | 116 | 36.998 | −9.390 | 25.115 | 1.00 | 47.83 | O |
| ATOM | 1791 | N | LYS | A | 117 | 38.080 | −9.235 | 23.139 | 1.00 | 62.79 | N |
| ATOM | 1793 | CA | LYS | A | 117 | 39.450 | −9.213 | 23.661 | 1.00 | 69.59 | C |
| ATOM | 1795 | CB | LYS | A | 117 | 40.431 | −8.650 | 22.609 | 1.00 | 78.18 | C |
| ATOM | 1798 | CG | LYS | A | 117 | 41.949 | −8.767 | 22.946 | 1.00 | 88.17 | C |
| ATOM | 1801 | CD | LYS | A | 117 | 42.851 | −8.730 | 21.685 | 1.00 | 89.30 | C |
| ATOM | 1804 | CE | LYS | A | 117 | 44.343 | −8.529 | 21.975 | 1.00 | 86.95 | C |
| ATOM | 1807 | NZ | LYS | A | 117 | 44.843 | −7.174 | 21.560 | 1.00 | 82.55 | N |
| ATOM | 1811 | C | LYS | A | 117 | 39.539 | −8.399 | 24.933 | 1.00 | 67.91 | C |
| ATOM | 1812 | O | LYS | A | 117 | 39.651 | −8.958 | 26.012 | 1.00 | 74.93 | O |
| ATOM | 1813 | N | LEU | A | 118 | 39.470 | −7.082 | 24.809 | 1.00 | 68.40 | N |
| ATOM | 1815 | CA | LEU | A | 118 | 39.794 | −6.185 | 25.914 | 1.00 | 70.99 | C |
| ATOM | 1817 | CB | LEU | A | 118 | 39.403 | −6.813 | 27.246 | 1.00 | 70.12 | C |
| ATOM | 1820 | CG | LEU | A | 118 | 38.597 | −5.861 | 28.128 | 1.00 | 74.28 | C |
| ATOM | 1822 | CD1 | LEU | A | 118 | 37.329 | −5.407 | 27.417 | 1.00 | 71.99 | C |
| ATOM | 1826 | CD2 | LEU | A | 118 | 38.270 | −6.498 | 29.464 | 1.00 | 71.79 | C |
| ATOM | 1830 | C | LEU | A | 118 | 41.276 | −5.793 | 25.911 | 1.00 | 75.02 | C |
| ATOM | 1831 | O | LEU | A | 118 | 41.981 | −5.955 | 26.916 | 1.00 | 62.55 | O |
| ATOM | 1832 | N | MET | A | 119 | 41.724 | −5.285 | 24.761 | 1.00 | 83.82 | N |
| ATOM | 1834 | CA | MET | A | 119 | 43.123 | −4.933 | 24.518 | 1.00 | 90.13 | C |
| ATOM | 1836 | CB | MET | A | 119 | 43.376 | −4.746 | 23.011 | 1.00 | 90.16 | C |
| ATOM | 1839 | CG | MET | A | 119 | 42.558 | −3.641 | 22.327 | 1.00 | 94.99 | C |
| ATOM | 1842 | SD | MET | A | 119 | 42.558 | −3.700 | 20.495 | 1.00 | 105.24 | S |
| ATOM | 1843 | CE | MET | A | 119 | 42.125 | −1.995 | 20.071 | 1.00 | 98.03 | C |
| ATOM | 1847 | C | MET | A | 119 | 43.526 | −3.678 | 25.285 | 1.00 | 94.21 | C |
| ATOM | 1848 | O | MET | A | 119 | 43.371 | −2.570 | 24.790 | 1.00 | 94.15 | O |
| ATOM | 1849 | N | ASP | A | 120 | 44.042 | −3.867 | 26.497 | 1.00 | 103.42 | N |
| ATOM | 1851 | CA | ASP | A | 120 | 44.552 | −2.772 | 27.323 | 1.00 | 108.76 | C |
| ATOM | 1853 | CB | ASP | A | 120 | 44.855 | −3.294 | 28.738 | 1.00 | 111.29 | C |
| ATOM | 1856 | CG | ASP | A | 120 | 45.670 | −2.313 | 29.584 | 1.00 | 115.23 | C |
| ATOM | 1857 | OD1 | ASP | A | 120 | 46.872 | −2.581 | 29.812 | 1.00 | 114.78 | O |
| ATOM | 1858 | OD2 | ASP | A | 120 | 45.189 | −1.284 | 30.111 | 1.00 | 105.43 | O |
| ATOM | 1859 | C | ASP | A | 120 | 45.798 | −2.140 | 26.689 | 1.00 | 110.71 | C |
| ATOM | 1860 | O | ASP | A | 120 | 46.895 | −2.693 | 26.769 | 1.00 | 106.51 | O |
| ATOM | 1861 | N | LEU | A | 121 | 45.611 | −0.971 | 26.078 | 1.00 | 115.07 | N |
| ATOM | 1863 | CA | LEU | A | 121 | 46.619 | −0.344 | 25.221 | 1.00 | 119.06 | C |
| ATOM | 1865 | CB | LEU | A | 121 | 46.119 | −0.303 | 23.762 | 1.00 | 119.09 | C |
| ATOM | 1868 | CG | LEU | A | 121 | 47.113 | −0.399 | 22.589 | 1.00 | 114.27 | C |
| ATOM | 1870 | CD1 | LEU | A | 121 | 48.316 | −1.254 | 22.955 | 1.00 | 111.21 | C |
| ATOM | 1874 | CD2 | LEU | A | 121 | 46.462 | −0.918 | 21.297 | 1.00 | 103.60 | C |
| ATOM | 1878 | C | LEU | A | 121 | 46.958 | 1.072 | 25.725 | 1.00 | 123.25 | C |
| ATOM | 1879 | O | LEU | A | 121 | 46.182 | 1.682 | 26.469 | 1.00 | 123.24 | O |
| ATOM | 1880 | N | ASP | A | 122 | 48.133 | 1.568 | 25.331 | 1.00 | 126.10 | N |
| ATOM | 1882 | CA | ASP | A | 122 | 48.627 | 2.893 | 25.723 | 1.00 | 126.10 | C |
| ATOM | 1884 | CB | ASP | A | 122 | 49.989 | 3.148 | 25.060 | 1.00 | 125.13 | C |
| ATOM | 1887 | CG | ASP | A | 122 | 51.154 | 2.599 | 25.863 | 1.00 | 120.01 | C |
| ATOM | 1888 | OD1 | ASP | A | 122 | 52.179 | 3.307 | 25.963 | 1.00 | 99.90 | O |
| ATOM | 1889 | OD2 | ASP | A | 122 | 51.149 | 1.473 | 26.406 | 1.00 | 114.22 | O |
| ATOM | 1890 | C | ASP | A | 122 | 47.666 | 3.995 | 25.274 | 1.00 | 127.08 | C |
| ATOM | 1891 | O | ASP | A | 122 | 47.492 | 4.209 | 24.075 | 1.00 | 126.93 | O |
| ATOM | 1892 | N | VAL | A | 123 | 47.070 | 4.719 | 26.216 | 1.00 | 127.79 | N |
| ATOM | 1894 | CA | VAL | A | 123 | 45.992 | 5.638 | 25.861 | 1.00 | 129.83 | C |
| ATOM | 1896 | CB | VAL | A | 123 | 44.616 | 4.953 | 26.041 | 1.00 | 130.96 | C |
| ATOM | 1898 | CG1 | VAL | A | 123 | 44.123 | 5.054 | 27.489 | 1.00 | 131.56 | C |
| ATOM | 1902 | CG2 | VAL | A | 123 | 43.602 | 5.504 | 25.042 | 1.00 | 130.17 | C |
| ATOM | 1906 | C | VAL | A | 123 | 46.050 | 6.973 | 26.609 | 1.00 | 130.26 | C |
| ATOM | 1907 | O | VAL | A | 123 | 47.117 | 7.411 | 27.039 | 1.00 | 128.58 | O |
| ATOM | 1908 | N | GLU | A | 124 | 44.903 | 7.633 | 26.734 | 1.00 | 131.74 | N |
| ATOM | 1910 | CA | GLU | A | 124 | 44.841 | 8.998 | 27.249 | 1.00 | 132.17 | C |
| ATOM | 1912 | CB | GLU | A | 124 | 44.382 | 9.942 | 26.123 | 1.00 | 132.91 | C |
| ATOM | 1915 | CG | GLU | A | 124 | 44.511 | 9.364 | 24.712 | 1.00 | 136.94 | C |
| ATOM | 1918 | CD | GLU | A | 124 | 43.177 | 9.210 | 23.988 | 1.00 | 138.68 | C |
| ATOM | 1919 | OE1 | GLU | A | 124 | 42.887 | 10.006 | 23.069 | 1.00 | 128.50 | O |
| ATOM | 1920 | OE2 | GLU | A | 124 | 42.416 | 8.274 | 24.314 | 1.00 | 142.29 | O |
| ATOM | 1921 | C | GLU | A | 124 | 43.875 | 9.073 | 28.444 | 1.00 | 131.07 | C |
| ATOM | 1922 | O | GLU | A | 124 | 43.420 | 8.030 | 28.955 | 1.00 | 132.02 | O |
| ATOM | 1923 | N | GLN | A | 125 | 43.594 | 10.298 | 28.905 | 1.00 | 129.38 | N |
| ATOM | 1925 | CA | GLN | A | 125 | 42.462 | 10.565 | 29.798 | 1.00 | 129.24 | C |
| ATOM | 1927 | CB | GLN | A | 125 | 42.572 | 9.765 | 31.106 | 1.00 | 130.65 | C |
| ATOM | 1930 | CG | GLN | A | 125 | 41.760 | 8.463 | 31.141 | 1.00 | 134.42 | C |
| ATOM | 1933 | CD | GLN | A | 125 | 40.497 | 8.552 | 31.990 | 1.00 | 133.87 | C |
| ATOM | 1934 | OE1 | GLN | A | 125 | 40.565 | 8.711 | 33.211 | 1.00 | 137.44 | O |
| ATOM | 1935 | NE2 | GLN | A | 125 | 39.342 | 8.430 | 31.345 | 1.00 | 121.15 | N |
| ATOM | 1938 | C | GLN | A | 125 | 42.384 | 12.059 | 30.116 | 1.00 | 128.13 | C |
| ATOM | 1939 | O | GLN | A | 125 | 42.975 | 12.524 | 31.089 | 1.00 | 126.81 | O |
| ATOM | 1940 | N | LEU | A | 126 | 41.632 | 12.802 | 29.307 | 1.00 | 128.67 | N |
| ATOM | 1942 | CA | LEU | A | 126 | 41.642 | 14.263 | 29.355 | 1.00 | 128.65 | C |
| ATOM | 1944 | CB | LEU | A | 126 | 41.493 | 14.830 | 27.938 | 1.00 | 128.80 | C |
| ATOM | 1947 | CG | LEU | A | 126 | 41.397 | 16.351 | 27.779 | 1.00 | 129.85 | C |
| ATOM | 1949 | CD1 | LEU | A | 126 | 42.520 | 17.063 | 28.533 | 1.00 | 125.66 | C |
| ATOM | 1953 | CD2 | LEU | A | 126 | 41.412 | 16.713 | 26.294 | 1.00 | 129.31 | C |
| ATOM | 1957 | C | LEU | A | 126 | 40.555 | 14.829 | 30.273 | 1.00 | 128.29 | C |
| ATOM | 1958 | O | LEU | A | 126 | 40.805 | 15.088 | 31.450 | 1.00 | 128.12 | O |
| ATOM | 1959 | N | GLY | A | 127 | 39.356 | 15.033 | 29.733 | 1.00 | 127.42 | N |
| ATOM | 1961 | CA | GLY | A | 127 | 38.284 | 15.683 | 30.467 | 1.00 | 125.14 | C |
| ATOM | 1964 | C | GLY | A | 127 | 38.014 | 17.085 | 29.953 | 1.00 | 124.07 | C |
| ATOM | 1965 | O | GLY | A | 127 | 36.956 | 17.359 | 29.382 | 1.00 | 118.81 | O |
| ATOM | 1966 | N | ILE | A | 128 | 38.993 | 17.966 | 30.138 | 1.00 | 125.14 | N |
| ATOM | 1968 | CA | ILE | A | 128 | 38.830 | 19.398 | 29.878 | 1.00 | 127.68 | C |
| ATOM | 1970 | CB | ILE | A | 128 | 38.650 | 19.670 | 28.360 | 1.00 | 129.42 | C |
| ATOM | 1972 | CG1 | ILE | A | 128 | 39.679 | 20.701 | 27.872 | 1.00 | 127.51 | C |
| ATOM | 1975 | CD1 | ILE | A | 128 | 41.131 | 20.260 | 28.000 | 1.00 | 123.72 | C |
| ATOM | 1979 | CG2 | ILE | A | 128 | 37.224 | 20.140 | 28.045 | 1.00 | 129.25 | C |
| ATOM | 1983 | C | ILE | A | 128 | 37.688 | 20.001 | 30.706 | 1.00 | 127.98 | C |
| ATOM | 1984 | O | ILE | A | 128 | 36.580 | 19.457 | 30.750 | 1.00 | 128.65 | O |
| ATOM | 1985 | N | PRO | A | 129 | 37.970 | 21.118 | 31.376 | 1.00 | 126.24 | N |
| ATOM | 1986 | CA | PRO | A | 129 | 37.126 | 21.598 | 32.477 | 1.00 | 124.56 | C |
| ATOM | 1988 | CB | PRO | A | 129 | 37.957 | 22.733 | 33.090 | 1.00 | 126.55 | C |
| ATOM | 1991 | CG | PRO | A | 129 | 38.847 | 23.204 | 31.992 | 1.00 | 127.23 | C |
| ATOM | 1994 | CD | PRO | A | 129 | 39.125 | 22.000 | 31.137 | 1.00 | 126.36 | C |
| ATOM | 1997 | C | PRO | A | 129 | 35.756 | 22.114 | 32.042 | 1.00 | 121.53 | C |
| ATOM | 1998 | O | PRO | A | 129 | 35.435 | 22.130 | 30.851 | 1.00 | 121.30 | O |
| ATOM | 1999 | N | GLU | A | 130 | 34.952 | 22.519 | 33.021 | 1.00 | 116.16 | N |
| ATOM | 2001 | CA | GLU | A | 130 | 33.689 | 23.188 | 32.740 | 1.00 | 111.91 | C |
| ATOM | 2003 | CB | GLU | A | 130 | 32.850 | 23.316 | 34.019 | 1.00 | 112.52 | C |
| ATOM | 2006 | CG | GLU | A | 130 | 31.343 | 23.206 | 33.807 | 1.00 | 111.51 | C |
| ATOM | 2009 | CD | GLU | A | 130 | 30.956 | 22.036 | 32.918 | 1.00 | 107.80 | C |
| ATOM | 2010 | OE1 | GLU | A | 130 | 31.389 | 20.902 | 33.215 | 1.00 | 106.50 | O |
| ATOM | 2011 | OE2 | GLU | A | 130 | 30.234 | 22.252 | 31.918 | 1.00 | 94.30 | O |
| ATOM | 2012 | C | GLU | A | 130 | 33.992 | 24.563 | 32.147 | 1.00 | 105.83 | C |
| ATOM | 2013 | O | GLU | A | 130 | 34.862 | 25.278 | 32.646 | 1.00 | 105.74 | O |
| ATOM | 2014 | N | GLN | A | 131 | 33.314 | 24.911 | 31.056 | 1.00 | 98.12 | N |
| ATOM | 2016 | CA | GLN | A | 131 | 33.462 | 26.240 | 30.466 | 1.00 | 91.93 | C |
| ATOM | 2018 | CB | GLN | A | 131 | 33.941 | 26.165 | 29.007 | 1.00 | 90.39 | C |
| ATOM | 2021 | CG | GLN | A | 131 | 34.644 | 24.854 | 28.636 | 1.00 | 91.98 | C |
| ATOM | 2024 | CD | GLN | A | 131 | 35.921 | 25.052 | 27.832 | 1.00 | 98.22 | C |
| ATOM | 2025 | OE1 | GLN | A | 131 | 35.975 | 25.899 | 26.941 | 1.00 | 101.57 | O |
| ATOM | 2026 | NE2 | GLN | A | 131 | 36.949 | 24.262 | 28.140 | 1.00 | 93.79 | N |
| ATOM | 2029 | C | GLN | A | 131 | 32.147 | 27.002 | 30.573 | 1.00 | 85.94 | C |
| ATOM | 2030 | O | GLN | A | 131 | 31.096 | 26.419 | 30.845 | 1.00 | 82.95 | O |
| ATOM | 2031 | N | GLU | A | 132 | 32.222 | 28.319 | 30.418 | 1.00 | 82.77 | N |
| ATOM | 2033 | CA | GLU | A | 132 | 31.024 | 29.131 | 30.274 | 1.00 | 76.97 | C |
| ATOM | 2035 | CB | GLU | A | 132 | 31.076 | 30.379 | 31.165 | 1.00 | 79.54 | C |
| ATOM | 2038 | CG | GLU | A | 132 | 29.767 | 31.163 | 31.230 | 1.00 | 88.49 | C |
| ATOM | 2041 | CD | GLU | A | 132 | 28.920 | 30.829 | 32.450 | 1.00 | 97.89 | C |
| ATOM | 2042 | OE1 | GLU | A | 132 | 28.930 | 31.618 | 33.420 | 1.00 | 100.91 | O |
| ATOM | 2043 | OE2 | GLU | A | 132 | 28.225 | 29.788 | 32.437 | 1.00 | 100.48 | O |
| ATOM | 2044 | C | GLU | A | 132 | 30.951 | 29.516 | 28.814 | 1.00 | 65.02 | C |
| ATOM | 2045 | O | GLU | A | 132 | 31.914 | 30.011 | 28.246 | 1.00 | 67.61 | O |
| ATOM | 2046 | N | TYR | A | 133 | 29.799 | 29.270 | 28.206 | 1.00 | 62.54 | N |
| ATOM | 2048 | CA | TYR | A | 133 | 29.647 | 29.449 | 26.769 | 1.00 | 56.85 | C |
| ATOM | 2050 | CB | TYR | A | 133 | 28.928 | 28.213 | 26.222 | 1.00 | 58.90 | C |
| ATOM | 2053 | CG | TYR | A | 133 | 29.800 | 26.973 | 26.256 | 1.00 | 44.13 | C |
| ATOM | 2054 | CD1 | TYR | A | 133 | 29.728 | 26.074 | 27.298 | 1.00 | 50.53 | C |
| ATOM | 2056 | CE1 | TYR | A | 133 | 30.549 | 24.968 | 27.340 | 1.00 | 48.72 | C |
| ATOM | 2058 | CZ | TYR | A | 133 | 31.457 | 24.748 | 26.325 | 1.00 | 42.43 | C |
| ATOM | 2059 | OH | TYR | A | 133 | 32.280 | 23.632 | 26.335 | 1.00 | 45.45 | O |
| ATOM | 2061 | CE2 | TYR | A | 133 | 31.526 | 25.618 | 25.268 | 1.00 | 38.56 | C |
| ATOM | 2063 | CD2 | TYR | A | 133 | 30.725 | 26.737 | 25.254 | 1.00 | 44.35 | C |
| ATOM | 2065 | C | TYR | A | 133 | 28.938 | 30.767 | 26.399 | 1.00 | 53.83 | C |
| ATOM | 2066 | O | TYR | A | 133 | 28.116 | 31.276 | 27.144 | 1.00 | 57.66 | O |
| ATOM | 2067 | N | SER | A | 134 | 29.285 | 31.330 | 25.250 | 1.00 | 56.69 | N |
| ATOM | 2069 | CA | SER | A | 134 | 28.653 | 32.544 | 24.735 | 1.00 | 57.68 | C |
| ATOM | 2071 | CB | SER | A | 134 | 29.449 | 33.052 | 23.523 | 1.00 | 59.77 | C |
| ATOM | 2074 | OG | SER | A | 134 | 30.594 | 33.777 | 23.926 | 1.00 | 57.88 | O |
| ATOM | 2076 | C | SER | A | 134 | 27.219 | 32.272 | 24.273 | 1.00 | 58.48 | C |
| ATOM | 2077 | O | SER | A | 134 | 26.327 | 33.113 | 24.414 | 1.00 | 49.23 | O |
| ATOM | 2078 | N | CYS | A | 135 | 27.028 | 31.114 | 23.647 | 1.00 | 53.77 | N |
| ATOM | 2080 | CA | CYS | A | 135 | 25.701 | 30.683 | 23.259 | 1.00 | 50.24 | C |
| ATOM | 2082 | CB | CYS | A | 135 | 25.493 | 30.876 | 21.766 | 1.00 | 53.73 | C |
| ATOM | 2085 | SG | CYS | A | 135 | 25.456 | 32.613 | 21.278 | 1.00 | 62.29 | S |
| ATOM | 2086 | C | CYS | A | 135 | 25.463 | 29.232 | 23.617 | 1.00 | 48.38 | C |
| ATOM | 2087 | O | CYS | A | 135 | 26.357 | 28.393 | 23.515 | 1.00 | 49.37 | O |
| ATOM | 2088 | N | VAL | A | 136 | 24.233 | 28.953 | 24.019 | 1.00 | 44.52 | N |
| ATOM | 2090 | CA | VAL | A | 136 | 23.734 | 27.599 | 24.128 | 1.00 | 46.71 | C |
| ATOM | 2092 | CB | VAL | A | 136 | 23.584 | 27.190 | 25.618 | 1.00 | 47.03 | C |
| ATOM | 2094 | CG1 | VAL | A | 136 | 23.170 | 25.748 | 25.759 | 1.00 | 45.82 | C |
| ATOM | 2098 | CG2 | VAL | A | 136 | 24.889 | 27.418 | 26.355 | 1.00 | 41.27 | C |
| ATOM | 2102 | C | VAL | A | 136 | 22.392 | 27.559 | 23.384 | 1.00 | 49.49 | C |
| ATOM | 2103 | O | VAL | A | 136 | 21.467 | 28.323 | 23.689 | 1.00 | 49.61 | O |
| ATOM | 2104 | N | VAL | A | 137 | 22.320 | 26.674 | 22.389 | 1.00 | 49.19 | N |
| ATOM | 2106 | CA | VAL | A | 137 | 21.123 | 26.450 | 21.588 | 1.00 | 39.09 | C |
| ATOM | 2108 | CB | VAL | A | 137 | 21.420 | 26.488 | 20.061 | 1.00 | 41.52 | C |
| ATOM | 2110 | CG1 | VAL | A | 137 | 20.208 | 26.027 | 19.247 | 1.00 | 43.08 | C |
| ATOM | 2114 | CG2 | VAL | A | 137 | 21.858 | 27.872 | 19.590 | 1.00 | 35.47 | C |
| ATOM | 2118 | C | VAL | A | 137 | 20.587 | 25.071 | 21.933 | 1.00 | 36.55 | C |
| ATOM | 2119 | O | VAL | A | 137 | 21.329 | 24.094 | 21.882 | 1.00 | 39.52 | O |
| ATOM | 2120 | N | LYS | A | 138 | 19.301 | 25.007 | 22.298 | 1.00 | 39.72 | N |
| ATOM | 2122 | CA | LYS | A | 138 | 18.560 | 23.759 | 22.371 | 1.00 | 32.48 | C |
| ATOM | 2124 | CB | LYS | A | 138 | 17.928 | 23.601 | 23.746 | 1.00 | 47.14 | C |
| ATOM | 2127 | CG | LYS | A | 138 | 17.036 | 22.354 | 23.887 | 1.00 | 45.49 | C |
| ATOM | 2130 | CD | LYS | A | 138 | 16.953 | 21.919 | 25.360 | 1.00 | 54.16 | C |
| ATOM | 2133 | CE | LYS | A | 138 | 15.890 | 20.839 | 25.574 | 1.00 | 67.79 | C |
| ATOM | 2136 | NZ | LYS | A | 138 | 14.549 | 21.318 | 25.089 | 1.00 | 63.82 | N |
| ATOM | 2140 | C | LYS | A | 138 | 17.499 | 23.651 | 21.276 | 1.00 | 31.63 | C |
| ATOM | 2141 | O | LYS | A | 138 | 16.819 | 24.612 | 20.957 | 1.00 | 31.25 | O |
| ATOM | 2142 | N | MET | A | 139 | 17.362 | 22.466 | 20.691 | 1.00 | 30.38 | N |
| ATOM | 2144 | CA | MET | A | 139 | 16.530 | 22.286 | 19.528 | 1.00 | 27.54 | C |
| ATOM | 2146 | CB | MET | A | 139 | 17.245 | 22.892 | 18.312 | 1.00 | 40.35 | C |
| ATOM | 2149 | CG | MET | A | 139 | 18.516 | 22.159 | 17.867 | 1.00 | 29.00 | C |
| ATOM | 2152 | SD | MET | A | 139 | 19.011 | 22.673 | 16.160 | 1.00 | 37.79 | S |
| ATOM | 2153 | CE | MET | A | 139 | 19.983 | 23.886 | 16.535 | 1.00 | 33.63 | C |
| ATOM | 2157 | C | MET | A | 139 | 16.303 | 20.798 | 19.320 | 1.00 | 29.56 | C |
| ATOM | 2158 | O | MET | A | 139 | 16.963 | 19.942 | 19.938 | 1.00 | 26.42 | O |
| ATOM | 2159 | N | PRO | A | 140 | 15.308 | 20.487 | 18.499 | 1.00 | 22.95 | N |
| ATOM | 2160 | CA | PRO | A | 140 | 15.005 | 19.093 | 18.215 | 1.00 | 25.35 | C |
| ATOM | 2162 | CB | PRO | A | 140 | 13.782 | 19.165 | 17.283 | 1.00 | 30.21 | C |
| ATOM | 2165 | CG | PRO | A | 140 | 13.224 | 20.566 | 17.493 | 1.00 | 31.58 | C |
| ATOM | 2168 | CD | PRO | A | 140 | 14.441 | 21.420 | 17.754 | 1.00 | 32.21 | C |
| ATOM | 2171 | C | PRO | A | 140 | 16.191 | 18.459 | 17.501 | 1.00 | 26.86 | C |
| ATOM | 2172 | O | PRO | A | 140 | 16.729 | 18.990 | 16.508 | 1.00 | 29.75 | O |
| ATOM | 2173 | N | SER | A | 141 | 16.457 | 17.223 | 17.908 | 1.00 | 28.84 | N |
| ATOM | 2175 | CA | SER | A | 141 | 17.595 | 16.485 | 17.377 | 1.00 | 28.80 | C |
| ATOM | 2177 | CB | SER | A | 141 | 17.742 | 15.205 | 18.146 | 1.00 | 26.96 | C |
| ATOM | 2180 | OG | SER | A | 141 | 16.528 | 14.481 | 18.177 | 1.00 | 33.86 | O |
| ATOM | 2182 | C | SER | A | 141 | 17.410 | 16.144 | 15.921 | 1.00 | 34.59 | C |
| ATOM | 2183 | O | SER | A | 141 | 18.369 | 16.108 | 15.175 | 1.00 | 33.55 | O |
| ATOM | 2184 | N | GLY | A | 142 | 16.173 | 15.870 | 15.521 | 1.00 | 35.90 | N |
| ATOM | 2186 | CA | GLY | A | 142 | 15.906 | 15.421 | 14.173 | 1.00 | 28.51 | C |
| ATOM | 2189 | C | GLY | A | 142 | 16.048 | 16.602 | 13.258 | 1.00 | 29.16 | C |
| ATOM | 2190 | O | GLY | A | 142 | 16.435 | 16.442 | 12.107 | 1.00 | 29.30 | O |
| ATOM | 2191 | N | GLU | A | 143 | 15.711 | 17.778 | 13.760 | 1.00 | 22.32 | N |
| ATOM | 2193 | CA | GLU | A | 143 | 15.941 | 19.003 | 13.008 | 1.00 | 27.01 | C |
| ATOM | 2195 | CB | GLU | A | 143 | 15.267 | 20.146 | 13.727 | 1.00 | 18.55 | C |
| ATOM | 2198 | CG | GLU | A | 143 | 15.406 | 21.474 | 13.006 | 1.00 | 44.67 | C |
| ATOM | 2201 | CD | GLU | A | 143 | 14.752 | 21.452 | 11.623 | 1.00 | 56.54 | C |
| ATOM | 2202 | OE1 | GLU | A | 143 | 13.522 | 21.266 | 11.576 | 1.00 | 60.92 | O |
| ATOM | 2203 | OE2 | GLU | A | 143 | 15.448 | 21.624 | 10.588 | 1.00 | 55.25 | O |
| ATOM | 2204 | C | GLU | A | 143 | 17.434 | 19.380 | 12.773 | 1.00 | 27.49 | C |
| ATOM | 2205 | O | GLU | A | 143 | 17.790 | 19.924 | 11.726 | 1.00 | 25.54 | O |
| ATOM | 2206 | N | PHE | A | 144 | 18.285 | 19.152 | 13.762 | 1.00 | 22.85 | N |
| ATOM | 2208 | CA | PHE | A | 144 | 19.704 | 19.424 | 13.608 | 1.00 | 28.92 | C |
| ATOM | 2210 | CB | PHE | A | 144 | 20.345 | 19.349 | 14.985 | 1.00 | 30.11 | C |
| ATOM | 2213 | CG | PHE | A | 144 | 21.807 | 19.590 | 14.991 | 1.00 | 31.59 | C |
| ATOM | 2214 | CD1 | PHE | A | 144 | 22.319 | 20.830 | 14.657 | 1.00 | 38.92 | C |
| ATOM | 2216 | CE1 | PHE | A | 144 | 23.697 | 21.056 | 14.691 | 1.00 | 32.51 | C |
| ATOM | 2218 | CZ | PHE | A | 144 | 24.573 | 20.042 | 15.062 | 1.00 | 29.35 | C |
| ATOM | 2220 | CE2 | PHE | A | 144 | 24.082 | 18.784 | 15.341 | 1.00 | 35.99 | C |
| ATOM | 2222 | CD2 | PHE | A | 144 | 22.690 | 18.568 | 15.331 | 1.00 | 43.00 | C |
| ATOM | 2224 | C | PHE | A | 144 | 20.276 | 18.389 | 12.653 | 1.00 | 21.81 | C |
| ATOM | 2225 | O | PHE | A | 144 | 21.102 | 18.709 | 11.810 | 1.00 | 28.22 | O |
| ATOM | 2226 | N | ALA | A | 145 | 19.791 | 17.150 | 12.740 | 1.00 | 21.05 | N |
| ATOM | 2228 | CA | ALA | A | 145 | 20.236 | 16.134 | 11.817 | 1.00 | 25.84 | C |
| ATOM | 2230 | CB | ALA | A | 145 | 19.575 | 14.751 | 12.129 | 1.00 | 30.36 | C |
| ATOM | 2234 | C | ALA | A | 145 | 19.946 | 16.523 | 10.382 | 1.00 | 27.39 | C |
| ATOM | 2235 | O | ALA | A | 145 | 20.744 | 16.276 | 9.475 | 1.00 | 29.47 | O |
| ATOM | 2236 | N | ARG | A | 146 | 18.726 | 16.976 | 10.147 | 1.00 | 27.28 | N |
| ATOM | 2238 | CA | ARG | A | 146 | 18.327 | 17.402 | 8.811 | 1.00 | 29.42 | C |
| ATOM | 2240 | CB | ARG | A | 146 | 16.817 | 17.645 | 8.769 | 1.00 | 32.60 | C |
| ATOM | 2243 | CG | ARG | A | 146 | 16.232 | 18.166 | 7.450 | 1.00 | 48.23 | C |
| ATOM | 2246 | CD | ARG | A | 146 | 15.534 | 17.129 | 6.568 | 1.00 | 68.76 | C |
| ATOM | 2249 | NE | ARG | A | 146 | 14.375 | 17.716 | 5.879 | 1.00 | 91.58 | N |
| ATOM | 2251 | CZ | ARG | A | 146 | 14.279 | 17.951 | 4.568 | 1.00 | 83.12 | C |
| ATOM | 2252 | NH1 | ARG | A | 146 | 15.276 | 17.654 | 3.738 | 1.00 | 71.90 | N |
| ATOM | 2255 | NH2 | ARG | A | 146 | 13.169 | 18.501 | 4.092 | 1.00 | 79.74 | N |
| ATOM | 2258 | C | ARG | A | 146 | 19.166 | 18.577 | 8.255 | 1.00 | 27.04 | C |
| ATOM | 2259 | O | ARG | A | 146 | 19.517 | 18.542 | 7.103 | 1.00 | 26.06 | O |
| ATOM | 2260 | N | ILE | A | 147 | 19.470 | 19.607 | 9.048 | 1.00 | 29.95 | N |
| ATOM | 2262 | CA | ILE | A | 147 | 20.226 | 20.760 | 8.567 | 1.00 | 22.40 | C |
| ATOM | 2264 | CB | ILE | A | 147 | 20.258 | 21.821 | 9.682 | 1.00 | 23.71 | C |
| ATOM | 2266 | CG1 | ILE | A | 147 | 18.883 | 22.434 | 9.898 | 1.00 | 22.83 | C |
| ATOM | 2269 | CD1 | ILE | A | 147 | 18.806 | 23.282 | 11.118 | 1.00 | 39.06 | C |
| ATOM | 2273 | CG2 | ILE | A | 147 | 21.248 | 22.858 | 9.370 | 1.00 | 33.80 | C |
| ATOM | 2277 | C | ILE | A | 147 | 21.662 | 20.371 | 8.168 | 1.00 | 25.30 | C |
| ATOM | 2278 | O | ILE | A | 147 | 22.135 | 20.765 | 7.114 | 1.00 | 23.00 | O |
| ATOM | 2279 | N | CYS | A | 148 | 22.285 | 19.498 | 8.952 | 1.00 | 26.39 | N |
| ATOM | 2281 | CA | CYS | A | 148 | 23.636 | 18.976 | 8.694 | 1.00 | 28.41 | C |
| ATOM | 2283 | CB | CYS | A | 148 | 24.168 | 18.190 | 9.907 | 1.00 | 34.38 | C |
| ATOM | 2286 | SG | CYS | A | 148 | 24.517 | 19.288 | 11.330 | 1.00 | 31.74 | S |
| ATOM | 2287 | C | CYS | A | 148 | 23.680 | 18.104 | 7.462 | 1.00 | 24.99 | C |
| ATOM | 2288 | O | CYS | A | 148 | 24.589 | 18.237 | 6.632 | 1.00 | 31.08 | O |
| ATOM | 2289 | N | ARG | A | 149 | 22.668 | 17.274 | 7.294 | 1.00 | 31.04 | N |
| ATOM | 2291 | CA | ARG | A | 149 | 22.539 | 16.460 | 6.087 | 1.00 | 39.08 | C |
| ATOM | 2293 | CB | ARG | A | 149 | 21.398 | 15.442 | 6.218 | 1.00 | 38.90 | C |
| ATOM | 2296 | CG | ARG | A | 149 | 21.023 | 14.741 | 4.902 | 1.00 | 51.47 | C |
| ATOM | 2299 | CD | ARG | A | 149 | 19.662 | 14.071 | 4.950 | 1.00 | 61.96 | C |
| ATOM | 2302 | NE | ARG | A | 249 | 19.445 | 13.545 | 6.297 | 1.00 | 88.23 | N |
| ATOM | 2304 | CZ | ARG | A | 149 | 18.309 | 13.631 | 6.987 | 1.00 | 83.14 | C |
| ATOM | 2305 | NH1 | ARG | A | 149 | 17.224 | 14.185 | 6.452 | 1.00 | 93.01 | N |
| ATOM | 2308 | NH2 | ARG | A | 149 | 18.263 | 13.147 | 8.223 | 1.00 | 58.38 | N |
| ATOM | 2311 | C | ARG | A | 149 | 22.365 | 17.294 | 4.817 | 1.00 | 36.47 | C |
| ATOM | 2312 | O | ARG | A | 149 | 23.116 | 17.128 | 3.875 | 1.00 | 30.72 | O |
| ATOM | 2313 | N | ASP | A | 150 | 21.406 | 18.213 | 4.812 | 1.00 | 36.06 | N |
| ATOM | 2315 | CA | ASP | A | 150 | 21.142 | 19.037 | 3.627 | 1.00 | 30.08 | C |
| ATOM | 2317 | CB | ASP | A | 150 | 19.925 | 19.867 | 3.908 | 1.00 | 28.90 | C |
| ATOM | 2320 | CG | ASP | A | 150 | 18.648 | 19.023 | 4.016 | 1.00 | 46.57 | C |
| ATOM | 2321 | OD1 | ASP | A | 150 | 18.602 | 17.793 | 3.728 | 1.00 | 34.21 | O |
| ATOM | 2322 | OD2 | ASP | A | 150 | 17.598 | 19.540 | 4.403 | 1.00 | 29.72 | O |
| ATOM | 2323 | C | ASP | A | 150 | 22.331 | 19.953 | 3.242 | 1.00 | 32.20 | C |
| ATOM | 2324 | O | ASP | A | 150 | 22.709 | 20.045 | 2.060 | 1.00 | 27.19 | O |
| ATOM | 2325 | N | LEU | A | 151 | 22.948 | 20.602 | 4.224 | 1.00 | 20.36 | N |
| ATOM | 2327 | CA | LEU | A | 151 | 24.051 | 21.497 | 3.927 | 1.00 | 28.60 | C |
| ATOM | 2329 | CB | LEU | A | 151 | 24.385 | 22.314 | 5.152 | 1.00 | 20.15 | C |
| ATOM | 2332 | CG | LEU | A | 151 | 23.337 | 23.383 | 5.429 | 1.00 | 29.47 | C |
| ATOM | 2334 | CD1 | LEU | A | 151 | 23.892 | 24.247 | 6.550 | 1.00 | 30.47 | C |
| ATOM | 2338 | CD2 | LEU | A | 151 | 23.027 | 24.251 | 4.192 | 1.00 | 26.89 | C |
| ATOM | 2342 | C | LEU | A | 151 | 25.291 | 20.779 | 3.384 | 1.00 | 28.24 | C |
| ATOM | 2343 | O | LEU | A | 151 | 26.129 | 21.389 | 2.727 | 1.00 | 40.11 | O |
| ATOM | 2344 | N | SER | A | 152 | 25.354 | 19.467 | 3.597 | 1.00 | 32.46 | N |
| ATOM | 2346 | CA | SER | A | 152 | 26.520 | 18.680 | 3.245 | 1.00 | 34.58 | C |
| ATOM | 2348 | CB | SER | A | 152 | 26.489 | 17.361 | 4.000 | 1.00 | 32.50 | C |
| ATOM | 2351 | OG | SER | A | 152 | 25.548 | 16.537 | 3.336 | 1.00 | 40.17 | O |
| ATOM | 2353 | C | SER | A | 152 | 26.478 | 18.449 | 1.728 | 1.00 | 37.97 | C |
| ATOM | 2354 | O | SER | A | 152 | 27.497 | 18.140 | 1.128 | 1.00 | 42.40 | O |
| ATOM | 2355 | N | HIS | A | 153 | 25.311 | 18.648 | 1.109 | 1.00 | 34.61 | N |
| ATOM | 2357 | CA | HIS | A | 153 | 25.170 | 18.737 | −0.365 | 1.00 | 37.58 | C |
| ATOM | 2359 | CB | HIS | A | 153 | 23.696 | 18.707 | −0.823 | 1.00 | 42.67 | C |
| ATOM | 2362 | CG | HIS | A | 153 | 22.907 | 17.506 | −0.390 | 1.00 | 59.40 | C |
| ATOM | 2363 | ND1 | HIS | A | 153 | 23.470 | 16.413 | 0.230 | 1.00 | 69.84 | N |
| ATOM | 2365 | CE1 | HIS | A | 153 | 22.529 | 15.519 | 0.481 | 1.00 | 71.01 | C |
| ATOM | 2367 | NE2 | HIS | A | 153 | 21.380 | 15.984 | 0.028 | 1.00 | 72.46 | N |
| ATOM | 2369 | CD2 | HIS | A | 153 | 21.588 | 17.223 | −0.525 | 1.00 | 62.35 | C |
| ATOM | 2371 | C | HIS | A | 153 | 25.751 | 19.986 | −1.015 | 1.00 | 29.74 | C |
| ATOM | 2372 | O | HIS | A | 153 | 25.829 | 20.068 | −2.241 | 1.00 | 45.78 | O |
| ATOM | 2373 | N | ILE | A | 154 | 26.066 | 20.987 | −0.207 | 1.00 | 34.58 | N |
| ATOM | 2375 | CA | ILE | A | 154 | 26.408 | 22.322 | −0.678 | 1.00 | 33.89 | C |
| ATOM | 2377 | CB | ILE | A | 154 | 25.616 | 23.374 | 0.120 | 1.00 | 31.44 | C |
| ATOM | 2379 | CG1 | ILE | A | 154 | 24.139 | 23.095 | −0.010 | 1.00 | 35.57 | C |
| ATOM | 2382 | CD1 | ILE | A | 154 | 23.569 | 23.619 | −1.327 | 1.00 | 46.77 | C |
| ATOM | 2386 | CG2 | ILE | A | 154 | 25.918 | 24.757 | −0.409 | 1.00 | 48.73 | C |
| ATOM | 2390 | C | ILE | A | 154 | 27.883 | 22.604 | −0.439 | 1.00 | 35.90 | C |
| ATOM | 2391 | O | ILE | A | 154 | 28.610 | 22.998 | −1.354 | 1.00 | 37.91 | O |
| ATOM | 2392 | N | GLY | A | 155 | 28.307 | 22.417 | 0.808 | 1.00 | 39.88 | N |
| ATOM | 2394 | CA | GLY | A | 155 | 29.712 | 22.556 | 1.151 | 1.00 | 41.34 | C |
| ATOM | 2397 | C | GLY | A | 155 | 30.260 | 21.528 | 2.120 | 1.00 | 35.69 | C |
| ATOM | 2398 | O | GLY | A | 155 | 29.584 | 20.593 | 2.555 | 1.00 | 41.72 | O |
| ATOM | 2399 | N | ASP | A | 156 | 31.463 | 21.786 | 2.585 | 1.00 | 31.55 | N |
| ATOM | 2401 | CA | ASP | A | 156 | 32.065 | 20.889 | 3.532 | 1.00 | 34.03 | C |
| ATOM | 2403 | CB | ASP | A | 156 | 33.355 | 20.285 | 2.974 | 1.00 | 38.86 | C |
| ATOM | 2406 | CG | ASP | A | 156 | 34.455 | 21.298 | 2.821 | 1.00 | 48.48 | C |
| ATOM | 2407 | OD1 | ASP | A | 156 | 35.010 | 21.399 | 1.698 | 1.00 | 75.68 | O |
| ATOM | 2408 | OD2 | ASP | A | 156 | 34.867 | 22.004 | 3.772 | 1.00 | 73.40 | O |
| ATOM | 2409 | C | ASP | A | 156 | 32.280 | 21.598 | 4.846 | 1.00 | 27.67 | C |
| ATOM | 2410 | O | ASP | A | 156 | 32.693 | 20.992 | 5.807 | 1.00 | 32.63 | O |
| ATOM | 2411 | N | ALA | A | 157 | 31.880 | 22.853 | 4.922 | 1.00 | 33.72 | N |
| ATOM | 2413 | CA | ALA | A | 157 | 31.907 | 23.575 | 6.180 | 1.00 | 34.38 | C |
| ATOM | 2415 | CB | ALA | A | 157 | 33.123 | 24.520 | 6.172 | 1.00 | 35.69 | C |
| ATOM | 2419 | C | ALA | A | 157 | 30.595 | 24.365 | 6.388 | 1.00 | 27.84 | C |
| ATOM | 2420 | O | ALA | A | 157 | 29.900 | 24.714 | 5.445 | 1.00 | 33.46 | O |
| ATOM | 2421 | N | VAL | A | 158 | 30.290 | 24.648 | 7.640 | 1.00 | 29.79 | N |
| ATOM | 2423 | CA | VAL | A | 158 | 29.100 | 25.392 | 8.025 | 1.00 | 27.85 | C |
| ATOM | 2425 | CB | VAL | A | 158 | 28.052 | 24.455 | 8.669 | 1.00 | 30.24 | C |
| ATOM | 2427 | CG1 | VAL | A | 158 | 28.662 | 23.637 | 9.812 | 1.00 | 29.24 | C |
| ATOM | 2431 | CG2 | VAL | A | 158 | 26.806 | 25.214 | 9.135 | 1.00 | 38.81 | C |
| ATOM | 2435 | C | VAL | A | 158 | 29.512 | 26.461 | 8.992 | 1.00 | 29.23 | C |
| ATOM | 2436 | O | VAL | A | 158 | 30.198 | 26.170 | 9.984 | 1.00 | 30.91 | O |
| ATOM | 2437 | N | VAL | A | 159 | 29.137 | 27.701 | 8.659 | 1.00 | 27.89 | N |
| ATOM | 2439 | CA | VAL | A | 159 | 29.164 | 28.791 | 9.604 | 1.00 | 30.14 | C |
| ATOM | 2441 | CB | VAL | A | 159 | 29.326 | 30.133 | 8.826 | 1.00 | 34.31 | C |
| ATOM | 2443 | CG1 | VAL | A | 159 | 29.544 | 31.266 | 9.802 | 1.00 | 41.80 | C |
| ATOM | 2447 | CG2 | VAL | A | 159 | 30.581 | 30.028 | 7.884 | 1.00 | 23.73 | C |
| ATOM | 2451 | C | VAL | A | 159 | 27.910 | 28.736 | 10.464 | 1.00 | 33.62 | C |
| ATOM | 2452 | O | VAL | A | 159 | 26.789 | 28.937 | 9.966 | 1.00 | 40.71 | O |
| ATOM | 2453 | N | ILE | A | 160 | 28.085 | 28.429 | 11.746 | 1.00 | 38.03 | N |
| ATOM | 2455 | CA | ILE | A | 160 | 27.054 | 28.729 | 12.749 | 1.00 | 36.04 | C |
| ATOM | 2457 | CB | ILE | A | 160 | 26.983 | 27.647 | 13.877 | 1.00 | 38.02 | C |
| ATOM | 2459 | CG1 | ILE | A | 160 | 26.852 | 26.253 | 13.231 | 1.00 | 31.10 | C |
| ATOM | 2462 | CD1 | ILE | A | 160 | 27.161 | 24.995 | 14.075 | 1.00 | 29.07 | C |
| ATOM | 2466 | CG2 | ILE | A | 160 | 25.719 | 27.942 | 14.744 | 1.00 | 36.48 | C |
| ATOM | 2470 | C | ILE | A | 160 | 27.146 | 30.126 | 13.358 | 1.00 | 36.34 | C |
| ATOM | 2471 | O | ILE | A | 160 | 28.107 | 30.421 | 14.062 | 1.00 | 42.45 | O |
| ATOM | 2472 | N | SER | A | 161 | 26.130 | 30.963 | 13.100 | 1.00 | 41.31 | N |
| ATOM | 2474 | CA | SER | A | 161 | 25.999 | 32.300 | 13.703 | 1.00 | 42.18 | C |
| ATOM | 2476 | CB | SER | A | 161 | 25.877 | 33.389 | 12.625 | 1.00 | 32.44 | C |
| ATOM | 2479 | OG | SER | A | 161 | 26.669 | 33.059 | 11.498 | 1.00 | 60.03 | O |
| ATOM | 2481 | C | SER | A | 161 | 24.793 | 32.433 | 14.651 | 1.00 | 45.89 | C |
| ATOM | 2482 | O | SER | A | 161 | 23.647 | 32.300 | 14.244 | 1.00 | 45.30 | O |
| ATOM | 2483 | N | CYS | A | 162 | 25.051 | 32.784 | 15.902 | 1.00 | 45.34 | N |
| ATOM | 2485 | CA | CYS | A | 162 | 24.001 | 32.860 | 16.891 | 1.00 | 54.06 | C |
| ATOM | 2487 | CB | CYS | A | 162 | 24.475 | 32.319 | 18.229 | 1.00 | 57.68 | C |
| ATOM | 2490 | SG | CYS | A | 162 | 24.167 | 30.582 | 18.509 | 1.00 | 79.58 | S |
| ATOM | 2491 | C | CYS | A | 162 | 23.640 | 34.310 | 17.093 | 1.00 | 54.41 | C |
| ATOM | 2492 | O | CYS | A | 162 | 24.460 | 35.213 | 16.921 | 1.00 | 57.06 | O |
| ATOM | 2493 | N | ALA | A | 163 | 22.404 | 34.509 | 17.513 | 1.00 | 50.61 | N |
| ATOM | 2495 | CA | ALA | A | 163 | 21.890 | 35.842 | 17.757 | 1.00 | 53.04 | C |
| ATOM | 2497 | CB | ALA | A | 163 | 21.585 | 36.520 | 16.430 | 1.00 | 52.10 | C |
| ATOM | 2501 | C | ALA | A | 163 | 20.625 | 35.711 | 18.612 | 1.00 | 54.40 | C |
| ATOM | 2502 | O | ALA | A | 163 | 20.097 | 34.615 | 18.780 | 1.00 | 53.51 | O |
| ATOM | 2503 | N | LYS | A | 164 | 20.144 | 36.838 | 19.129 | 1.00 | 60.05 | N |
| ATOM | 2505 | CA | LYS | A | 164 | 19.080 | 36.882 | 20.135 | 1.00 | 58.44 | C |
| ATOM | 2507 | CB | LYS | A | 164 | 18.725 | 38.356 | 20.389 | 1.00 | 65.69 | C |
| ATOM | 2510 | CG | LYS | A | 164 | 17.975 | 38.683 | 21.682 | 1.00 | 64.77 | C |
| ATOM | 2513 | CD | LYS | A | 164 | 16.578 | 39.238 | 21.381 | 1.00 | 68.26 | C |
| ATOM | 2516 | CE | LYS | A | 164 | 16.454 | 40.745 | 21.570 | 1.00 | 71.21 | C |
| ATOM | 2519 | NZ | LYS | A | 164 | 16.165 | 41.114 | 22.981 | 1.00 | 66.81 | N |
| ATOM | 2523 | C | LYS | A | 164 | 17.870 | 36.134 | 19.599 | 1.00 | 57.66 | C |
| ATOM | 2524 | O | LYS | A | 164 | 17.314 | 35.245 | 20.261 | 1.00 | 54.18 | O |
| ATOM | 2525 | N | ASP | A | 165 | 17.550 | 36.443 | 18.345 | 1.00 | 55.80 | N |
| ATOM | 2527 | CA | ASP | A | 165 | 16.308 | 36.032 | 17.722 | 1.00 | 60.46 | C |
| ATOM | 2529 | CB | ASP | A | 165 | 15.803 | 37.182 | 16.838 | 1.00 | 66.43 | C |
| ATOM | 2532 | CG | ASP | A | 165 | 15.255 | 38.374 | 17.639 | 1.00 | 83.13 | C |
| ATOM | 2533 | OD1 | ASP | A | 165 | 14.635 | 38.173 | 18.717 | 1.00 | 78.18 | O |
| ATOM | 2534 | OD2 | ASP | A | 165 | 15.361 | 39.558 | 17.228 | 1.00 | 90.20 | O |
| ATOM | 2535 | C | ASP | A | 165 | 16.440 | 34.766 | 16.853 | 1.00 | 58.66 | C |
| ATOM | 2536 | O | ASP | A | 165 | 15.577 | 34.497 | 16.028 | 1.00 | 56.67 | O |
| ATOM | 2537 | N | GLY | A | 166 | 17.512 | 33.997 | 16.995 | 1.00 | 54.85 | N |
| ATOM | 2539 | CA | GLY | A | 166 | 17.713 | 32.856 | 16.116 | 1.00 | 55.92 | C |
| ATOM | 2542 | C | GLY | A | 166 | 19.132 | 32.384 | 15.837 | 1.00 | 47.32 | C |
| ATOM | 2543 | O | GLY | A | 166 | 20.119 | 33.057 | 16.124 | 1.00 | 47.27 | O |
| ATOM | 2544 | N | VAL | A | 167 | 19.225 | 31.197 | 15.247 | 1.00 | 37.45 | N |
| ATOM | 2546 | CA | VAL | A | 167 | 20.496 | 30.679 | 14.813 | 1.00 | 37.88 | C |
| ATOM | 2548 | CB | VAL | A | 167 | 20.855 | 29.484 | 15.680 | 1.00 | 40.51 | C |
| ATOM | 2550 | CG1 | VAL | A | 167 | 19.677 | 28.567 | 15.766 | 1.00 | 41.53 | C |
| ATOM | 2554 | CG2 | VAL | A | 167 | 22.129 | 28.775 | 15.203 | 1.00 | 32.52 | C |
| ATOM | 2558 | C | VAL | A | 167 | 20.498 | 30.368 | 13.321 | 1.00 | 39.88 | C |
| ATOM | 2559 | O | VAL | A | 167 | 19.510 | 29.843 | 12.792 | 1.00 | 35.44 | O |
| ATOM | 2560 | N | LYS | A | 168 | 21.620 | 30.696 | 12.668 | 1.00 | 34.33 | N |
| ATOM | 2562 | CA | LYS | A | 168 | 21.817 | 30.474 | 11.236 | 1.00 | 40.41 | C |
| ATOM | 2564 | CB | LYS | A | 168 | 22.124 | 31.803 | 10.505 | 1.00 | 40.60 | C |
| ATOM | 2567 | CG | LYS | A | 168 | 22.174 | 31.706 | 8.981 | 1.00 | 39.02 | C |
| ATOM | 2570 | CD | LYS | A | 168 | 21.869 | 33.020 | 8.266 | 1.00 | 29.87 | C |
| ATOM | 2573 | CE | LYS | A | 168 | 22.924 | 34.070 | 8.586 | 1.00 | 50.17 | C |
| ATOM | 2576 | NZ | LYS | A | 168 | 23.319 | 34.909 | 7.414 | 1.00 | 50.90 | N |
| ATOM | 2580 | C | LYS | A | 168 | 22.967 | 29.497 | 10.961 | 1.00 | 40.74 | C |
| ATOM | 2581 | O | LYS | A | 168 | 23.967 | 29.478 | 11.680 | 1.00 | 36.06 | O |
| ATOM | 2582 | N | PHE | A | 169 | 22.794 | 28.729 | 9.884 | 1.00 | 36.34 | N |
| ATOM | 2584 | CA | PHE | A | 169 | 23.737 | 27.755 | 9.383 | 1.00 | 33.14 | C |
| ATOM | 2586 | CB | PHE | A | 169 | 23.255 | 26.317 | 9.553 | 1.00 | 32.70 | C |
| ATOM | 2589 | CG | PHE | A | 169 | 22.857 | 25.971 | 10.938 | 1.00 | 36.42 | C |
| ATOM | 2590 | CD1 | PHE | A | 169 | 21.614 | 26.333 | 11.432 | 1.00 | 47.04 | C |
| ATOM | 2592 | CE1 | PHE | A | 169 | 21.239 | 25.958 | 12.719 | 1.00 | 25.17 | C |
| ATOM | 2594 | CZ | PHE | A | 169 | 22.081 | 25.215 | 13.483 | 1.00 | 31.76 | C |
| ATOM | 2596 | CE2 | PHE | A | 169 | 23.306 | 24.817 | 12.979 | 1.00 | 43.70 | C |
| ATOM | 2598 | CD2 | PHE | A | 169 | 23.671 | 25.181 | 11.709 | 1.00 | 31.36 | C |
| ATOM | 2600 | C | PHE | A | 169 | 23.847 | 28.015 | 7.900 | 1.00 | 30.65 | C |
| ATOM | 2601 | O | PHE | A | 169 | 22.878 | 27.876 | 7.164 | 1.00 | 35.33 | O |
| ATOM | 2602 | N | SER | A | 170 | 25.074 | 28.309 | 7.480 | 1.00 | 34.56 | N |
| ATOM | 2604 | CA | SER | A | 170 | 25.377 | 28.750 | 6.149 | 1.00 | 29.58 | C |
| ATOM | 2606 | CB | SER | A | 170 | 25.683 | 30.247 | 6.234 | 1.00 | 25.55 | C |
| ATOM | 2609 | OG | SER | A | 170 | 26.737 | 30.582 | 5.341 | 1.00 | 57.38 | O |
| ATOM | 2611 | C | SER | A | 170 | 26.563 | 27.913 | 5.595 | 1.00 | 33.98 | C |
| ATOM | 2612 | O | SER | A | 170 | 27.506 | 27.604 | 6.302 | 1.00 | 36.26 | O |
| ATOM | 2613 | N | ALA | A | 171 | 26.509 | 27.560 | 4.319 | 1.00 | 30.61 | N |
| ATOM | 2615 | CA | ALA | A | 171 | 27.580 | 26.818 | 3.665 | 1.00 | 30.85 | C |
| ATOM | 2617 | CB | ALA | A | 171 | 27.419 | 25.255 | 3.823 | 1.00 | 29.03 | C |
| ATOM | 2621 | C | ALA | A | 171 | 27.650 | 27.211 | 2.198 | 1.00 | 36.55 | C |
| ATOM | 2622 | O | ALA | A | 171 | 26.720 | 27.787 | 1.605 | 1.00 | 30.97 | O |
| ATOM | 2623 | N | SER | A | 172 | 28.796 | 26.894 | 1.613 | 1.00 | 38.66 | N |
| ATOM | 2625 | CA | SER | A | 172 | 29.169 | 27.411 | 0.318 | 1.00 | 35.51 | C |
| ATOM | 2627 | CB | SER | A | 172 | 29.988 | 28.683 | 0.509 | 1.00 | 36.90 | C |
| ATOM | 2630 | OG | SER | A | 172 | 29.748 | 29.529 | −0.572 | 1.00 | 49.14 | O |
| ATOM | 2632 | C | SER | A | 172 | 30.064 | 26.436 | −0.396 | 1.00 | 37.55 | C |
| ATOM | 2633 | O | SER | A | 172 | 30.987 | 25.918 | 0.196 | 1.00 | 39.16 | O |
| ATOM | 2634 | N | GLY | A | 173 | 29.896 | 26.345 | −1.711 | 1.00 | 34.49 | N |
| ATOM | 2636 | CA | GLY | A | 173 | 30.591 | 25.356 | −2.489 | 1.00 | 36.73 | C |
| ATOM | 2639 | C | GLY | A | 173 | 30.263 | 25.389 | −3.953 | 1.00 | 36.94 | C |
| ATOM | 2640 | O | GLY | A | 173 | 29.988 | 26.420 | −4.559 | 1.00 | 46.36 | O |
| ATOM | 2641 | N | GLU | A | 174 | 30.297 | 24.200 | −4.517 | 1.00 | 42.46 | N |
| ATOM | 2643 | CA | GLU | A | 174 | 30.440 | 24.004 | −5.948 | 1.00 | 47.43 | C |
| ATOM | 2645 | CB | GLU | A | 174 | 30.691 | 22.509 | −6.190 | 1.00 | 45.37 | C |
| ATOM | 2648 | CG | GLU | A | 174 | 32.093 | 21.989 | −5.832 | 1.00 | 75.56 | C |
| ATOM | 2651 | CD | GLU | A | 174 | 32.375 | 21.686 | −4.351 | 1.00 | 87.43 | C |
| ATOM | 2652 | OE1 | GLU | A | 174 | 31.452 | 21.698 | −3.488 | 1.00 | 69.20 | O |
| ATOM | 2653 | OE2 | GLU | A | 174 | 33.573 | 21.425 | −4.052 | 1.00 | 90.71 | O |
| ATOM | 2654 | C | GLU | A | 174 | 29.135 | 24.425 | −6.637 | 1.00 | 48.69 | C |
| ATOM | 2655 | O | GLU | A | 174 | 29.103 | 25.162 | −7.638 | 1.00 | 52.48 | O |
| ATOM | 2656 | N | LEU | A | 175 | 28.033 | 23.931 | −6.102 | 1.00 | 42.52 | N |
| ATOM | 2658 | CA | LEU | A | 175 | 26.774 | 24.197 | −6.748 | 1.00 | 47.07 | C |
| ATOM | 2660 | CB | LEU | A | 175 | 25.836 | 22.992 | −6.648 | 1.00 | 52.01 | C |
| ATOM | 2663 | CG | LEU | A | 175 | 25.226 | 22.696 | −5.298 | 1.00 | 51.60 | C |
| ATOM | 2665 | CD1 | LEU | A | 175 | 24.048 | 23.635 | −5.102 | 1.00 | 67.44 | C |
| ATOM | 2669 | CD2 | LEU | A | 175 | 24.833 | 21.230 | −5.221 | 1.00 | 51.79 | C |
| ATOM | 2673 | C | LEU | A | 175 | 26.106 | 25.522 | −6.378 | 1.00 | 36.34 | C |
| ATOM | 2674 | O | LEU | A | 175 | 25.118 | 25.867 | −7.015 | 1.00 | 37.59 | O |
| ATOM | 2675 | N | GLY | A | 176 | 26.719 | 26.287 | −5.472 | 1.00 | 25.76 | N |
| ATOM | 2677 | CA | GLY | A | 176 | 26.247 | 27.587 | −4.999 | 1.00 | 29.02 | C |
| ATOM | 2680 | C | GLY | A | 176 | 26.339 | 27.683 | −3.484 | 1.00 | 33.93 | C |
| ATOM | 2681 | O | GLY | A | 176 | 27.197 | 26.990 | −2.893 | 1.00 | 37.34 | O |
| ATOM | 2682 | N | ASN | A | 177 | 25.480 | 28.501 | −2.858 | 1.00 | 30.46 | N |
| ATOM | 2684 | CA | ASN | A | 177 | 25.442 | 28.657 | −1.397 | 1.00 | 31.09 | C |
| ATOM | 2686 | CB | ASN | A | 177 | 26.214 | 29.885 | −0.949 | 1.00 | 38.32 | C |
| ATOM | 2689 | CG | ASN | A | 177 | 25.570 | 31.188 | −1.358 | 1.00 | 52.66 | C |
| ATOM | 2690 | OD1 | ASN | A | 177 | 24.673 | 31.701 | −0.685 | 1.00 | 51.08 | O |
| ATOM | 2691 | ND2 | ASN | A | 177 | 26.102 | 31.786 | −2.427 | 1.00 | 59.21 | N |
| ATOM | 2694 | C | ASN | A | 177 | 24.056 | 28.614 | −0.720 | 1.00 | 27.02 | C |
| ATOM | 2695 | O | ASN | A | 177 | 23.045 | 28.509 | −1.385 | 1.00 | 29.26 | O |
| ATOM | 2696 | N | GLY | A | 178 | 24.013 | 28.631 | 0.606 | 1.00 | 34.44 | N |
| ATOM | 2698 | CA | GLY | A | 178 | 22.860 | 28.146 | 1.340 | 1.00 | 35.17 | C |
| ATOM | 2701 | C | GLY | A | 178 | 22.873 | 28.729 | 2.730 | 1.00 | 33.27 | C |
| ATOM | 2702 | O | GLY | A | 178 | 23.930 | 28.716 | 3.318 | 1.00 | 27.41 | O |
| ATOM | 2703 | N | ASN | A | 179 | 21.735 | 29.223 | 3.238 | 1.00 | 33.22 | N |
| ATOM | 2705 | CA | ASN | A | 179 | 21.545 | 29.470 | 4.673 | 1.00 | 31.50 | C |
| ATOM | 2707 | CB | ASN | A | 179 | 21.639 | 30.961 | 4.996 | 1.00 | 33.52 | C |
| ATOM | 2710 | CG | ASN | A | 179 | 21.867 | 31.822 | 3.780 | 1.00 | 45.24 | C |
| ATOM | 2711 | OD1 | ASN | A | 179 | 20.977 | 32.560 | 3.385 | 1.00 | 70.99 | O |
| ATOM | 2712 | ND2 | ASN | A | 179 | 23.070 | 31.766 | 3.199 | 1.00 | 48.47 | N |
| ATOM | 2715 | C | ASN | A | 179 | 20.179 | 28.980 | 5.174 | 1.00 | 32.83 | C |
| ATOM | 2716 | O | ASN | A | 179 | 19.147 | 29.285 | 4.576 | 1.00 | 31.94 | O |
| ATOM | 2717 | N | ILE | A | 180 | 20.192 | 28.347 | 6.347 | 1.00 | 33.83 | N |
| ATOM | 2719 | CA | ILE | A | 180 | 18.999 | 27.924 | 7.054 | 1.00 | 29.31 | C |
| ATOM | 2721 | CB | ILE | A | 180 | 19.076 | 26.427 | 7.328 | 1.00 | 22.65 | C |
| ATOM | 2723 | CG1 | ILE | A | 180 | 19.389 | 25.723 | 5.986 | 1.00 | 19.89 | C |
| ATOM | 2726 | CD1 | ILE | A | 180 | 19.892 | 24.310 | 6.140 | 1.00 | 29.06 | C |
| ATOM | 2730 | CG2 | ILE | A | 180 | 17.741 | 25.924 | 7.949 | 1.00 | 33.89 | C |
| ATOM | 2734 | C | ILE | A | 180 | 18.927 | 28.688 | 8.352 | 1.00 | 34.00 | C |
| ATOM | 2735 | O | ILE | A | 180 | 19.786 | 28.538 | 9.197 | 1.00 | 43.43 | O |
| ATOM | 2736 | N | LYS | A | 181 | 17.912 | 29.530 | 8.470 | 1.00 | 29.47 | N |
| ATOM | 2738 | CA | LYS | A | 181 | 17.618 | 30.316 | 9.668 | 1.00 | 28.64 | C |
| ATOM | 2740 | CB | LYS | A | 181 | 17.197 | 31.726 | 9.187 | 1.00 | 29.15 | C |
| ATOM | 2743 | CG | LYS | A | 181 | 17.712 | 32.917 | 10.005 | 1.00 | 51.08 | C |
| ATOM | 2746 | CD | LYS | A | 181 | 17.431 | 32.772 | 11.533 | 1.00 | 57.56 | C |
| ATOM | 2749 | CE | LYS | A | 181 | 17.505 | 34.107 | 12.289 | 1.00 | 62.24 | C |
| ATOM | 2752 | NZ | LYS | A | 181 | 18.892 | 34.478 | 12.722 | 1.00 | 57.93 | N |
| ATOM | 2756 | C | LYS | A | 181 | 16.468 | 29.591 | 10.429 | 1.00 | 36.00 | C |
| ATOM | 2757 | O | LYS | A | 181 | 15.400 | 29.329 | 9.842 | 1.00 | 32.17 | O |
| ATOM | 2758 | N | LEU | A | 182 | 16.751 | 29.141 | 11.657 | 1.00 | 40.60 | N |
| ATOM | 2760 | CA | LEU | A | 182 | 15.717 | 28.796 | 12.651 | 1.00 | 43.51 | C |
| ATOM | 2762 | CB | LEU | A | 182 | 16.081 | 27.602 | 13.528 | 1.00 | 40.14 | C |
| ATOM | 2765 | CG | LEU | A | 182 | 16.315 | 26.207 | 12.991 | 1.00 | 50.93 | C |
| ATOM | 2767 | CD1 | LEU | A | 182 | 16.767 | 25.434 | 14.197 | 1.00 | 46.49 | C |
| ATOM | 2771 | CD2 | LEU | A | 182 | 15.048 | 25.630 | 12.403 | 1.00 | 56.87 | C |
| ATOM | 2775 | C | LEU | A | 182 | 15.506 | 29.909 | 13.658 | 1.00 | 43.73 | C |
| ATOM | 2776 | O | LEU | A | 182 | 16.448 | 30.368 | 14.330 | 1.00 | 35.35 | O |
| ATOM | 2777 | N | SER | A | 183 | 14.229 | 30.168 | 13.882 | 1.00 | 39.11 | N |
| ATOM | 2779 | CA | SER | A | 183 | 13.764 | 31.250 | 14.736 | 1.00 | 47.87 | C |
| ATOM | 2781 | CB | SER | A | 183 | 12.343 | 31.667 | 14.308 | 1.00 | 52.05 | C |
| ATOM | 2784 | OG | SER | A | 183 | 12.307 | 32.127 | 12.959 | 1.00 | 59.22 | O |
| ATOM | 2786 | C | SER | A | 183 | 13.700 | 30.675 | 16.135 | 1.00 | 46.83 | C |
| ATOM | 2787 | O | SER | A | 183 | 13.322 | 29.523 | 16.297 | 1.00 | 34.54 | O |
| ATOM | 2788 | N | GLN | A | 184 | 14.073 | 31.470 | 17.130 | 1.00 | 45.68 | N |
| ATOM | 2790 | CA | GLN | A | 184 | 13.658 | 31.221 | 18.495 | 1.00 | 53.26 | C |
| ATOM | 2792 | CB | GLN | A | 184 | 13.904 | 32.457 | 19.365 | 1.00 | 50.31 | C |
| ATOM | 2795 | CG | GLN | A | 184 | 15.089 | 32.370 | 20.278 | 1.00 | 69.15 | C |
| ATOM | 2798 | CD | GLN | A | 184 | 14.669 | 32.324 | 21.728 | 1.00 | 86.67 | C |
| ATOM | 2799 | OE1 | GLN | A | 184 | 15.381 | 32.816 | 22.604 | 1.00 | 85.37 | O |
| ATOM | 2800 | NE2 | GLN | A | 184 | 13.505 | 31.732 | 21.986 | 1.00 | 95.85 | N |
| ATOM | 2803 | C | GLN | A | 184 | 12.165 | 30.956 | 18.441 | 1.00 | 49.03 | C |
| ATOM | 2804 | O | GLN | A | 184 | 11.418 | 31.734 | 17.876 | 1.00 | 58.91 | O |
| ATOM | 2805 | N | THR | A | 185 | 11.724 | 29.865 | 19.040 | 1.00 | 54.55 | N |
| ATOM | 2807 | CA | THR | A | 185 | 10.313 | 29.729 | 19.352 | 1.00 | 61.64 | C |
| ATOM | 2809 | CB | THR | A | 185 | 9.639 | 28.670 | 18.476 | 1.00 | 54.57 | C |
| ATOM | 2811 | OG1 | THR | A | 185 | 10.258 | 27.412 | 18.722 | 1.00 | 49.43 | O |
| ATOM | 2813 | CG2 | THR | A | 185 | 9.884 | 28.913 | 16.998 | 1.00 | 61.50 | C |
| ATOM | 2817 | C | THR | A | 185 | 10.209 | 29.328 | 20.809 | 1.00 | 70.19 | C |
| ATOM | 2818 | O | THR | A | 185 | 11.093 | 28.671 | 21.346 | 1.00 | 72.69 | O |
| ATOM | 2819 | N | SER | A | 186 | 9.113 | 29.717 | 21.440 | 1.00 | 78.99 | N |
| ATOM | 2821 | CA | SER | A | 186 | 8.998 | 29.598 | 22.882 | 1.00 | 86.27 | C |
| ATOM | 2823 | CB | SER | A | 186 | 8.306 | 30.848 | 23.438 | 1.00 | 88.12 | C |
| ATOM | 2826 | OG | SER | A | 186 | 8.754 | 32.035 | 22.793 | 1.00 | 94.33 | O |
| ATOM | 2828 | C | SER | A | 186 | 8.151 | 28.377 | 23.190 | 1.00 | 93.28 | C |
| ATOM | 2829 | O | SER | A | 186 | 8.661 | 27.320 | 23.588 | 1.00 | 87.15 | O |
| ATOM | 2830 | N | ASN | A | 187 | 6.856 | 28.568 | 22.930 | 1.00 | 102.30 | N |
| ATOM | 2832 | CA | ASN | A | 187 | 5.757 | 27.715 | 23.385 | 1.00 | 108.33 | C |
| ATOM | 2834 | CB | ASN | A | 187 | 5.278 | 26.799 | 22.256 | 1.00 | 105.24 | C |
| ATOM | 2837 | CG | ASN | A | 187 | 3.993 | 27.300 | 21.620 | 1.00 | 103.64 | C |
| ATOM | 2838 | OD1 | ASN | A | 187 | 3.037 | 26.545 | 21.472 | 1.00 | 101.98 | O |
| ATOM | 2839 | ND2 | ASN | A | 187 | 3.949 | 28.590 | 21.285 | 1.00 | 90.70 | N |
| ATOM | 2842 | C | ASN | A | 187 | 5.972 | 26.964 | 24.698 | 1.00 | 112.87 | C |
| ATOM | 2843 | O | ASN | A | 187 | 5.646 | 27.487 | 25.768 | 1.00 | 110.18 | O |
| ATOM | 2844 | N | VAL | A | 188 | 6.511 | 25.750 | 24.615 | 1.00 | 119.37 | N |
| ATOM | 2846 | CA | VAL | A | 188 | 6.694 | 24.906 | 25.793 | 1.00 | 125.15 | C |
| ATOM | 2848 | CB | VAL | A | 188 | 7.659 | 25.566 | 26.826 | 1.00 | 126.91 | C |
| ATOM | 2850 | CG1 | VAL | A | 188 | 8.044 | 24.580 | 27.936 | 1.00 | 125.49 | C |
| ATOM | 2854 | CG2 | VAL | A | 188 | 8.914 | 26.110 | 26.130 | 1.00 | 125.58 | C |
| ATOM | 2858 | C | VAL | A | 188 | 5.324 | 24.619 | 26.423 | 1.00 | 128.36 | C |
| ATOM | 2859 | O | VAL | A | 188 | 4.355 | 25.340 | 26.155 | 1.00 | 129.29 | O |
| ATOM | 2860 | N | ASP | A | 189 | 5.251 | 23.549 | 27.220 | 1.00 | 129.56 | N |
| ATOM | 2862 | CA | ASP | A | 189 | 4.012 | 23.068 | 27.851 | 1.00 | 129.06 | C |
| ATOM | 2864 | CB | ASP | A | 189 | 2.917 | 24.153 | 27.871 | 1.00 | 129.52 | C |
| ATOM | 2867 | CG | ASP | A | 189 | 2.792 | 24.850 | 29.225 | 1.00 | 122.48 | C |
| ATOM | 2868 | OD1 | ASP | A | 189 | 2.142 | 25.920 | 29.287 | 1.00 | 110.27 | O |
| ATOM | 2869 | OD2 | ASP | A | 189 | 3.298 | 24.397 | 30.276 | 1.00 | 101.10 | O |
| ATOM | 2870 | C | ASP | A | 189 | 3.483 | 21.789 | 27.178 | 1.00 | 128.95 | C |
| ATOM | 2871 | O | ASP | A | 189 | 3.369 | 20.742 | 27.822 | 1.00 | 127.57 | O |
| ATOM | 2872 | N | LYS | A | 190 | 3.188 | 21.884 | 25.880 | 1.00 | 129.60 | N |
| ATOM | 2874 | CA | LYS | A | 190 | 2.555 | 20.808 | 25.103 | 1.00 | 127.01 | C |
| ATOM | 2876 | CB | LYS | A | 190 | 1.610 | 21.407 | 24.041 | 1.00 | 127.28 | C |
| ATOM | 2879 | CG | LYS | A | 190 | 1.190 | 22.875 | 24.277 | 1.00 | 123.26 | C |
| ATOM | 2882 | CD | LYS | A | 190 | 1.484 | 23.791 | 23.082 | 1.00 | 120.66 | C |
| ATOM | 2885 | CE | LYS | A | 190 | 2.888 | 23.579 | 22.486 | 1.00 | 121.99 | C |
| ATOM | 2888 | NZ | LYS | A | 190 | 3.902 | 24.611 | 22.873 | 1.00 | 110.68 | N |
| ATOM | 2892 | C | LYS | A | 190 | 3.612 | 19.917 | 24.427 | 1.00 | 123.91 | C |
| ATOM | 2893 | O | LYS | A | 190 | 3.400 | 18.715 | 24.223 | 1.00 | 121.77 | O |
| ATOM | 2894 | N | GLU | A | 191 | 4.726 | 20.545 | 24.049 | 1.00 | 119.66 | N |
| ATOM | 2896 | CA | GLU | A | 191 | 5.974 | 19.857 | 23.717 | 1.00 | 115.28 | C |
| ATOM | 2898 | CB | GLU | A | 191 | 5.869 | 19.183 | 22.334 | 1.00 | 116.37 | C |
| ATOM | 2901 | CG | GLU | A | 191 | 6.456 | 17.775 | 22.245 | 1.00 | 114.77 | C |
| ATOM | 2904 | CD | GLU | A | 191 | 5.408 | 16.689 | 22.029 | 1.00 | 115.37 | C |
| ATOM | 2905 | OE1 | GLU | A | 191 | 4.434 | 16.916 | 21.277 | 1.00 | 110.48 | O |
| ATOM | 2906 | OE2 | GLU | A | 191 | 5.564 | 15.588 | 22.601 | 1.00 | 110.95 | O |
| ATOM | 2907 | C | GLU | A | 191 | 7.164 | 20.844 | 23.765 | 1.00 | 108.46 | C |
| ATOM | 2908 | O | GLU | A | 191 | 7.007 | 22.062 | 23.609 | 1.00 | 103.72 | O |
| ATOM | 2909 | N | GLU | A | 192 | 8.350 | 20.302 | 24.029 | 1.00 | 101.17 | N |
| ATOM | 2911 | CA | GLU | A | 192 | 9.602 | 20.976 | 23.699 | 1.00 | 95.31 | C |
| ATOM | 2913 | CB | GLU | A | 192 | 10.619 | 20.883 | 24.844 | 1.00 | 97.75 | C |
| ATOM | 2916 | CG | GLU | A | 192 | 10.035 | 20.461 | 26.193 | 1.00 | 106.41 | C |
| ATOM | 2919 | CD | GLU | A | 192 | 9.904 | 21.597 | 27.199 | 1.00 | 103.27 | C |
| ATOM | 2920 | OE1 | GLU | A | 192 | 10.353 | 22.728 | 26.906 | 1.00 | 113.24 | O |
| ATOM | 2921 | OE2 | GLU | A | 192 | 9.347 | 21.352 | 28.292 | 1.00 | 93.34 | O |
| ATOM | 2922 | C | GLU | A | 192 | 10.158 | 20.385 | 22.398 | 1.00 | 86.63 | C |
| ATOM | 2923 | O | GLU | A | 192 | 11.108 | 19.589 | 22.390 | 1.00 | 84.24 | O |
| ATOM | 2924 | N | GLU | A | 193 | 9.465 | 20.728 | 21.312 | 1.00 | 74.62 | N |
| ATOM | 2926 | CA | GLU | A | 193 | 10.066 | 20.901 | 19.998 | 1.00 | 59.25 | C |
| ATOM | 2928 | CB | GLU | A | 193 | 9.123 | 20.408 | 18.879 | 1.00 | 65.27 | C |
| ATOM | 2931 | CG | GLU | A | 193 | 7.696 | 20.982 | 18.844 | 1.00 | 68.48 | C |
| ATOM | 2934 | CD | GLU | A | 193 | 6.941 | 20.738 | 17.524 | 1.00 | 86.22 | C |
| ATOM | 2935 | OE1 | GLU | A | 193 | 7.411 | 21.162 | 16.430 | 1.00 | 78.70 | O |
| ATOM | 2936 | OE2 | GLU | A | 193 | 5.828 | 20.157 | 17.572 | 1.00 | 79.74 | O |
| ATOM | 2937 | C | GLU | A | 193 | 10.430 | 22.371 | 19.782 | 1.00 | 46.70 | C |
| ATOM | 2938 | O | GLU | A | 193 | 10.470 | 22.831 | 18.660 | 1.00 | 40.42 | O |
| ATOM | 2939 | N | ALA | A | 194 | 10.734 | 23.105 | 20.843 | 1.00 | 45.52 | N |
| ATOM | 2941 | CA | ALA | A | 194 | 11.163 | 24.494 | 20.724 | 1.00 | 49.19 | C |
| ATOM | 2943 | CB | ALA | A | 194 | 10.897 | 25.217 | 22.024 | 1.00 | 47.18 | C |
| ATOM | 2947 | C | ALA | A | 194 | 12.637 | 24.679 | 20.299 | 1.00 | 45.24 | C |
| ATOM | 2948 | O | ALA | A | 194 | 13.449 | 23.749 | 20.369 | 1.00 | 43.70 | O |
| ATOM | 2949 | N | VAL | A | 195 | 12.962 | 25.881 | 19.838 | 1.00 | 36.40 | N |
| ATOM | 2951 | CA | VAL | A | 195 | 14.355 | 26.291 | 19.642 | 1.00 | 41.44 | C |
| ATOM | 2953 | CB | VAL | A | 195 | 14.606 | 26.743 | 18.180 | 1.00 | 30.60 | C |
| ATOM | 2955 | CG1 | VAL | A | 195 | 16.025 | 27.128 | 17.951 | 1.00 | 41.34 | C |
| ATOM | 2959 | CG2 | VAL | A | 195 | 14.209 | 25.634 | 17.232 | 1.00 | 26.99 | C |
| ATOM | 2963 | C | VAL | A | 195 | 14.609 | 27.430 | 20.614 | 1.00 | 41.06 | C |
| ATOM | 2964 | O | VAL | A | 195 | 14.008 | 28.499 | 20.487 | 1.00 | 44.45 | O |
| ATOM | 2965 | N | THR | A | 196 | 15.377 | 27.155 | 21.665 | 1.00 | 40.84 | N |
| ATOM | 2967 | CA | THR | A | 196 | 15.716 | 28.201 | 22.622 | 1.00 | 45.55 | C |
| ATOM | 2969 | CB | THR | A | 196 | 15.278 | 27.816 | 24.055 | 1.00 | 43.61 | C |
| ATOM | 2971 | OG1 | THR | A | 196 | 16.084 | 26.750 | 24.549 | 1.00 | 55.97 | O |
| ATOM | 2973 | CG2 | THR | A | 196 | 13.897 | 27.213 | 24.049 | 1.00 | 46.82 | C |
| ATOM | 2977 | C | THR | A | 196 | 17.195 | 28.596 | 22.533 | 1.00 | 45.25 | C |
| ATOM | 2978 | O | THR | A | 196 | 18.085 | 27.767 | 22.272 | 1.00 | 44.27 | O |
| ATOM | 2979 | N | ILE | A | 197 | 17.424 | 29.894 | 22.684 | 1.00 | 52.52 | N |
| ATOM | 2981 | CA | ILE | A | 197 | 18.766 | 30.466 | 22.751 | 1.00 | 58.34 | C |
| ATOM | 2983 | CB | ILE | A | 197 | 18.920 | 31.529 | 21.662 | 1.00 | 57.87 | C |
| ATOM | 2985 | CG1 | ILE | A | 197 | 18.159 | 31.109 | 20.395 | 1.00 | 64.66 | C |
| ATOM | 2988 | CD1 | ILE | A | 197 | 18.937 | 30.254 | 19.416 | 1.00 | 62.83 | C |
| ATOM | 2992 | CG2 | ILE | A | 197 | 20.386 | 31.818 | 21.390 | 1.00 | 70.70 | C |
| ATOM | 2996 | C | ILE | A | 197 | 19.085 | 31.068 | 24.125 | 1.00 | 57.54 | C |
| ATOM | 2997 | O | ILE | A | 197 | 18.222 | 31.613 | 24.815 | 1.00 | 63.59 | O |
| ATOM | 2998 | N | GLU | A | 198 | 20.328 | 30.889 | 24.544 | 1.00 | 56.62 | N |
| ATOM | 3000 | CA | GLU | A | 198 | 20.871 | 31.552 | 25.722 | 1.00 | 57.53 | C |
| ATOM | 3002 | CB | GLU | A | 198 | 21.206 | 30.534 | 26.823 | 1.00 | 62.30 | C |
| ATOM | 3005 | CG | GLU | A | 198 | 19.975 | 29.901 | 27.476 | 1.00 | 70.63 | C |
| ATOM | 3008 | CD | GLU | A | 198 | 20.301 | 28.767 | 28.447 | 1.00 | 71.98 | C |
| ATOM | 3009 | OE1 | GLU | A | 198 | 19.801 | 27.641 | 28.251 | 1.00 | 65.93 | O |
| ATOM | 3010 | OE2 | GLU | A | 198 | 21.010 | 29.001 | 29.447 | 1.00 | 76.11 | O |
| ATOM | 3011 | C | GLU | A | 198 | 22.130 | 32.253 | 25.227 | 1.00 | 55.08 | C |
| ATOM | 3012 | O | GLU | A | 198 | 23.139 | 31.624 | 24.934 | 1.00 | 50.32 | O |
| ATOM | 3013 | N | MET | A | 199 | 22.033 | 33.562 | 25.081 | 1.00 | 53.00 | N |
| ATOM | 3015 | CA | MET | A | 199 | 22.995 | 34.334 | 24.328 | 1.00 | 56.69 | C |
| ATOM | 3017 | CB | MET | A | 199 | 22.252 | 34.953 | 23.132 | 1.00 | 61.63 | C |
| ATOM | 3020 | CG | MET | A | 199 | 23.116 | 35.298 | 21.928 | 1.00 | 69.55 | C |
| ATOM | 3023 | SD | MET | A | 199 | 23.666 | 37.004 | 22.030 | 1.00 | 79.28 | S |
| ATOM | 3024 | CE | MET | A | 199 | 22.812 | 37.737 | 20.623 | 1.00 | 91.69 | C |
| ATOM | 3028 | C | MET | A | 199 | 23.559 | 35.422 | 25.254 | 1.00 | 54.12 | C |
| ATOM | 3029 | O | MET | A | 199 | 22.870 | 36.386 | 25.576 | 1.00 | 52.81 | O |
| ATOM | 3030 | N | ASN | A | 200 | 24.798 | 35.261 | 25.707 | 1.00 | 57.22 | N |
| ATOM | 3032 | CA | ASN | A | 200 | 25.533 | 36.368 | 26.331 | 1.00 | 57.98 | C |
| ATOM | 3034 | CB | ASN | A | 200 | 26.573 | 35.816 | 27.313 | 1.00 | 60.48 | C |
| ATOM | 3037 | CG | ASN | A | 200 | 25.942 | 35.246 | 28.593 | 1.00 | 73.34 | C |
| ATOM | 3038 | OD1 | ASN | A | 200 | 24.864 | 35.675 | 29.027 | 1.00 | 65.93 | O |
| ATOM | 3039 | ND2 | ASN | A | 200 | 26.614 | 34.265 | 29.195 | 1.00 | 78.00 | N |
| ATOM | 3042 | C | ASN | A | 200 | 26.198 | 37.331 | 25.333 | 1.00 | 54.85 | C |
| ATOM | 3043 | O | ASN | A | 200 | 26.182 | 38.562 | 25.487 | 1.00 | 54.27 | O |
| ATOM | 3044 | N | GLU | A | 201 | 26.833 | 36.776 | 24.316 | 1.00 | 54.19 | N |
| ATOM | 3046 | CA | GLU | A | 201 | 27.277 | 37.578 | 23.181 | 1.00 | 56.69 | C |
| ATOM | 3048 | CB | GLU | A | 201 | 28.737 | 38.037 | 23.333 | 1.00 | 56.83 | C |
| ATOM | 3051 | CG | GLU | A | 201 | 29.661 | 37.073 | 24.054 | 1.00 | 66.32 | C |
| ATOM | 3054 | CD | GLU | A | 201 | 30.963 | 37.725 | 24.519 | 1.00 | 87.43 | C |
| ATOM | 3055 | OE1 | GLU | A | 201 | 31.247 | 37.699 | 25.740 | 1.00 | 87.75 | O |
| ATOM | 3056 | OE2 | GLU | A | 201 | 31.716 | 38.256 | 23.669 | 1.00 | 78.49 | O |
| ATOM | 3057 | C | GLU | A | 201 | 27.142 | 36.709 | 21.939 | 1.00 | 56.02 | C |
| ATOM | 3058 | O | GLU | A | 201 | 27.334 | 35.501 | 22.008 | 1.00 | 56.43 | O |
| ATOM | 3059 | N | PRO | A | 202 | 26.835 | 37.318 | 20.804 | 1.00 | 48.63 | N |
| ATOM | 3060 | CA | PRO | A | 202 | 26.671 | 36.560 | 19.564 | 1.00 | 48.91 | C |
| ATOM | 3062 | CB | PRO | A | 202 | 26.384 | 37.639 | 18.514 | 1.00 | 46.55 | C |
| ATOM | 3065 | CG | PRO | A | 202 | 25.960 | 38.845 | 19.313 | 1.00 | 61.17 | C |
| ATOM | 3068 | CD | PRO | A | 202 | 26.743 | 38.772 | 20.596 | 1.00 | 54.11 | C |
| ATOM | 3071 | C | PRO | A | 202 | 27.956 | 35.821 | 19.217 | 1.00 | 46.17 | C |
| ATOM | 3072 | O | PRO | A | 202 | 29.061 | 36.291 | 19.513 | 1.00 | 45.71 | O |
| ATOM | 3073 | N | VAL | A | 203 | 27.811 | 34.668 | 18.583 | 1.00 | 43.09 | N |
| ATOM | 3075 | CA | VAL | A | 203 | 28.986 | 33.912 | 18.171 | 1.00 | 45.60 | C |
| ATOM | 3077 | CB | VAL | A | 203 | 29.115 | 32.611 | 18.957 | 1.00 | 49.57 | C |
| ATOM | 3079 | CG1 | VAL | A | 203 | 30.473 | 31.960 | 18.671 | 1.00 | 48.60 | C |
| ATOM | 3083 | CG2 | VAL | A | 203 | 28.986 | 32.875 | 20.467 | 1.00 | 51.57 | C |
| ATOM | 3087 | C | VAL | A | 203 | 28.956 | 33.662 | 16.675 | 1.00 | 41.76 | C |
| ATOM | 3088 | O | VAL | A | 203 | 27.901 | 33.693 | 16.052 | 1.00 | 52.75 | O |
| ATOM | 3089 | N | GLN | A | 204 | 30.111 | 33.517 | 16.056 | 1.00 | 46.58 | N |
| ATOM | 3091 | CA | GLN | A | 204 | 30.132 | 33.082 | 14.661 | 1.00 | 49.31 | C |
| ATOM | 3093 | CB | GLN | A | 204 | 30.040 | 34.283 | 13.737 | 1.00 | 45.04 | C |
| ATOM | 3096 | CG | GLN | A | 204 | 30.068 | 33.956 | 12.279 | 1.00 | 46.41 | C |
| ATOM | 3099 | CD | GLN | A | 204 | 29.702 | 35.147 | 11.430 | 1.00 | 67.57 | C |
| ATOM | 3100 | OE1 | GLN | A | 204 | 28.534 | 35.564 | 11.398 | 1.00 | 64.10 | O |
| ATOM | 3101 | NE2 | GLN | A | 204 | 30.699 | 35.722 | 10.760 | 1.00 | 57.87 | N |
| ATOM | 3104 | C | GLN | A | 204 | 31.355 | 32.225 | 14.337 | 1.00 | 46.39 | C |
| ATOM | 3105 | O | GLN | A | 204 | 32.462 | 32.754 | 14.226 | 1.00 | 51.24 | O |
| ATOM | 3106 | N | LEU | A | 205 | 31.117 | 30.919 | 14.177 | 1.00 | 42.19 | N |
| ATOM | 3108 | CA | LEU | A | 205 | 32.152 | 29.891 | 14.043 | 1.00 | 40.71 | C |
| ATOM | 3110 | CB | LEU | A | 205 | 32.192 | 29.053 | 15.327 | 1.00 | 45.10 | C |
| ATOM | 3113 | CG | LEU | A | 205 | 32.165 | 29.899 | 16.611 | 1.00 | 52.62 | C |
| ATOM | 3115 | CD1 | LEU | A | 205 | 31.656 | 29.198 | 17.856 | 1.00 | 55.56 | C |
| ATOM | 3119 | CD2 | LEU | A | 205 | 33.544 | 30.501 | 16.857 | 1.00 | 61.04 | C |
| ATOM | 3123 | C | LEU | A | 205 | 31.868 | 28.977 | 12.851 | 1.00 | 41.15 | C |
| ATOM | 3124 | O | LEU | A | 205 | 30.731 | 28.606 | 12.624 | 1.00 | 46.10 | O |
| ATOM | 3125 | N | THR | A | 206 | 32.923 | 28.606 | 12.135 | 1.00 | 38.79 | N |
| ATOM | 3127 | CA | THR | A | 206 | 32.953 | 27.595 | 11.087 | 1.00 | 35.30 | C |
| ATOM | 3129 | CB | THR | A | 206 | 33.979 | 28.051 | 10.049 | 1.00 | 35.40 | C |
| ATOM | 3131 | OG1 | THR | A | 206 | 33.643 | 29.360 | 9.619 | 1.00 | 33.19 | O |
| ATOM | 3133 | CG2 | THR | A | 206 | 33.939 | 27.247 | 8.757 | 1.00 | 37.83 | C |
| ATOM | 3137 | C | THR | A | 206 | 33.380 | 26.220 | 11.594 | 1.00 | 35.30 | C |
| ATOM | 3138 | O | THR | A | 206 | 34.299 | 26.092 | 12.409 | 1.00 | 39.79 | O |
| ATOM | 3139 | N | PHE | A | 207 | 32.678 | 25.188 | 11.137 | 1.00 | 37.65 | N |
| ATOM | 3141 | CA | PHE | A | 207 | 32.919 | 23.820 | 11.595 | 1.00 | 34.72 | C |
| ATOM | 3143 | CB | PHE | A | 207 | 31.846 | 23.384 | 12.593 | 1.00 | 37.32 | C |
| ATOM | 3146 | CG | PHE | A | 207 | 31.829 | 24.206 | 13.849 | 1.00 | 37.39 | C |
| ATOM | 3147 | CD1 | PHE | A | 207 | 32.826 | 24.036 | 14.789 | 1.00 | 38.54 | C |
| ATOM | 3149 | CE1 | PHE | A | 207 | 32.872 | 24.813 | 15.920 | 1.00 | 27.53 | C |
| ATOM | 3151 | CZ | PHE | A | 207 | 31.922 | 25.792 | 16.135 | 1.00 | 36.03 | C |
| ATOM | 3153 | CE2 | PHE | A | 207 | 30.927 | 25.998 | 15.182 | 1.00 | 47.52 | C |
| ATOM | 3155 | CD2 | PHE | A | 207 | 30.913 | 25.225 | 14.027 | 1.00 | 40.88 | C |
| ATOM | 3157 | C | PHE | A | 207 | 32.894 | 22.866 | 10.427 | 1.00 | 33.32 | C |
| ATOM | 3158 | O | PHE | A | 207 | 32.250 | 23.111 | 9.408 | 1.00 | 34.16 | O |
| ATOM | 3159 | N | ALA | A | 208 | 33.517 | 21.722 | 10.638 | 1.00 | 33.35 | N |
| ATOM | 3161 | CA | ALA | A | 208 | 33.566 | 20.664 | 9.643 | 1.00 | 30.47 | C |
| ATOM | 3163 | CB | ALA | A | 208 | 34.687 | 19.715 | 9.984 | 1.00 | 29.99 | C |
| ATOM | 3167 | C | ALA | A | 208 | 32.270 | 19.884 | 9.525 | 1.00 | 34.95 | C |
| ATOM | 3168 | O | ALA | A | 208 | 31.845 | 19.290 | 10.529 | 1.00 | 34.31 | O |
| ATOM | 3169 | N | LEU | A | 209 | 31.652 | 19.875 | 8.327 | 1.00 | 30.55 | N |
| ATOM | 3171 | CA | LEU | A | 209 | 30.276 | 19.351 | 8.186 | 1.00 | 31.35 | C |
| ATOM | 3173 | CB | LEU | A | 209 | 29.595 | 19.837 | 6.883 | 1.00 | 29.27 | C |
| ATOM | 3176 | CG | LEU | A | 209 | 28.516 | 20.935 | 7.054 | 1.00 | 32.09 | C |
| ATOM | 3178 | CD1 | LEU | A | 209 | 28.009 | 21.530 | 5.740 | 1.00 | 36.76 | C |
| ATOM | 3182 | CD2 | LEU | A | 209 | 27.278 | 20.424 | 7.865 | 1.00 | 31.34 | C |
| ATOM | 3186 | C | LEU | A | 209 | 30.246 | 17.831 | 8.342 | 1.00 | 36.01 | C |
| ATOM | 3187 | O | LEU | A | 209 | 29.291 | 17.238 | 8.915 | 1.00 | 25.78 | O |
| ATOM | 3188 | N | ARG | A | 210 | 31.316 | 17.183 | 7.874 | 1.00 | 21.82 | N |
| ATOM | 3190 | CA | ARG | A | 210 | 31.396 | 15.729 | 7.959 | 1.00 | 22.51 | C |
| ATOM | 3192 | CB | ARG | A | 210 | 32.481 | 15.105 | 7.067 | 1.00 | 29.60 | C |
| ATOM | 3195 | CG | ARG | A | 210 | 32.236 | 15.362 | 5.555 | 1.00 | 36.12 | C |
| ATOM | 3198 | CD | ARG | A | 210 | 33.225 | 14.730 | 4.582 | 1.00 | 44.29 | C |
| ATOM | 3201 | NE | ARG | A | 210 | 32.959 | 15.064 | 3.174 | 1.00 | 42.85 | N |
| ATOM | 3203 | CZ | ARG | A | 210 | 33.471 | 14.382 | 2.147 | 1.00 | 49.67 | C |
| ATOM | 3204 | NH1 | ARG | A | 210 | 34.342 | 13.406 | 2.368 | 1.00 | 53.51 | N |
| ATOM | 3207 | NH2 | ARG | A | 210 | 33.162 | 14.695 | 0.899 | 1.00 | 61.04 | N |
| ATOM | 3210 | C | ARG | A | 210 | 31.468 | 15.199 | 9.367 | 1.00 | 26.07 | C |
| ATOM | 3211 | O | ARG | A | 210 | 31.066 | 14.051 | 9.662 | 1.00 | 26.61 | O |
| ATOM | 3212 | N | TYR | A | 211 | 32.028 | 15.999 | 10.247 | 1.00 | 25.44 | N |
| ATOM | 3214 | CA | TYR | A | 211 | 32.104 | 15.564 | 11.623 | 1.00 | 30.41 | C |
| ATOM | 3216 | CB | TYR | A | 211 | 33.183 | 16.312 | 12.403 | 1.00 | 34.80 | C |
| ATOM | 3219 | CG | TYR | A | 211 | 34.582 | 15.717 | 12.165 | 1.00 | 32.39 | C |
| ATOM | 3220 | CD1 | TYR | A | 211 | 35.471 | 16.308 | 11.285 | 1.00 | 34.24 | C |
| ATOM | 3222 | CE1 | TYR | A | 211 | 36.732 | 15.771 | 11.067 | 1.00 | 39.51 | C |
| ATOM | 3224 | CZ | TYR | A | 211 | 37.084 | 14.595 | 11.685 | 1.00 | 43.83 | C |
| ATOM | 3225 | OH | TYR | A | 211 | 38.317 | 14.034 | 11.442 | 1.00 | 64.88 | O |
| ATOM | 3227 | CE2 | TYR | A | 211 | 36.206 | 13.966 | 12.533 | 1.00 | 36.63 | C |
| ATOM | 3229 | CD2 | TYR | A | 211 | 34.980 | 14.554 | 12.794 | 1.00 | 31.24 | C |
| ATOM | 3231 | C | TYR | A | 211 | 30.724 | 15.678 | 12.259 | 1.00 | 31.42 | C |
| ATOM | 3232 | O | TYR | A | 211 | 30.292 | 14.767 | 12.955 | 1.00 | 27.14 | O |
| ATOM | 3233 | N | LEU | A | 212 | 30.052 | 16.796 | 12.026 | 1.00 | 25.98 | N |
| ATOM | 3235 | CA | LEU | A | 212 | 28.721 | 17.010 | 12.563 | 1.00 | 37.93 | C |
| ATOM | 3237 | CB | LEU | A | 212 | 28.191 | 18.398 | 12.212 | 1.00 | 33.90 | C |
| ATOM | 3240 | CG | LEU | A | 212 | 28.936 | 19.557 | 12.887 | 1.00 | 33.02 | C |
| ATOM | 3242 | CD1 | LEU | A | 212 | 28.410 | 20.814 | 12.265 | 1.00 | 35.27 | C |
| ATOM | 3246 | CD2 | LEU | A | 212 | 28.694 | 19.587 | 14.416 | 1.00 | 31.34 | C |
| ATOM | 3250 | C | LEU | A | 212 | 27.788 | 15.923 | 12.060 | 1.00 | 37.18 | C |
| ATOM | 3251 | O | LEU | A | 212 | 26.959 | 15.461 | 12.853 | 1.00 | 40.42 | O |
| ATOM | 3252 | N | ASN | A | 213 | 27.985 | 15.449 | 10.819 | 1.00 | 30.24 | N |
| ATOM | 3254 | CA | ASN | A | 213 | 27.237 | 14.282 | 10.316 | 1.00 | 39.92 | C |
| ATOM | 3256 | CB | ASN | A | 213 | 27.421 | 14.057 | 8.801 | 1.00 | 36.38 | C |
| ATOM | 3259 | CG | ASN | A | 213 | 26.853 | 15.189 | 7.936 | 1.00 | 54.01 | C |
| ATOM | 3260 | OD1 | ASN | A | 213 | 25.741 | 15.640 | 8.133 | 1.00 | 36.54 | O |
| ATOM | 3261 | ND2 | ASN | A | 213 | 27.626 | 15.628 | 6.955 | 1.00 | 79.35 | N |
| ATOM | 3264 | C | ASN | A | 213 | 27.515 | 12.938 | 11.045 | 1.00 | 45.78 | C |
| ATOM | 3265 | O | ASN | A | 213 | 26.621 | 12.055 | 11.112 | 1.00 | 32.57 | O |
| ATOM | 3266 | N | PHE | A | 214 | 28.745 | 12.737 | 11.528 | 1.00 | 37.40 | N |
| ATOM | 3268 | CA | PHE | A | 214 | 28.984 | 11.632 | 12.466 | 1.00 | 40.35 | C |
| ATOM | 3270 | CB | PHE | A | 214 | 30.475 | 11.388 | 12.797 | 1.00 | 31.41 | C |
| ATOM | 3273 | CG | PHE | A | 214 | 31.336 | 11.028 | 11.608 | 1.00 | 43.25 | C |
| ATOM | 3274 | CD1 | PHE | A | 214 | 31.049 | 9.928 | 10.821 | 1.00 | 53.02 | C |
| ATOM | 3276 | CE1 | PHE | A | 214 | 31.839 | 9.609 | 9.728 | 1.00 | 52.10 | C |
| ATOM | 3278 | CZ | PHE | A | 214 | 32.970 | 10.364 | 9.432 | 1.00 | 57.23 | C |
| ATOM | 3280 | CE2 | PHE | A | 214 | 33.311 | 11.414 | 10.235 | 1.00 | 50.15 | C |
| ATOM | 3282 | CD2 | PHE | A | 214 | 32.474 | 11.768 | 11.313 | 1.00 | 44.02 | C |
| ATOM | 3284 | C | PHE | A | 214 | 28.180 | 11.827 | 13.761 | 1.00 | 24.70 | C |
| ATOM | 3285 | O | PHE | A | 214 | 27.515 | 10.917 | 14.232 | 1.00 | 36.80 | O |
| ATOM | 3286 | N | PHE | A | 215 | 28.234 | 13.017 | 14.339 | 1.00 | 20.80 | N |
| ATOM | 3288 | CA | PHE | A | 215 | 27.636 | 13.202 | 15.642 | 1.00 | 21.40 | C |
| ATOM | 3290 | CB | PHE | A | 215 | 27.821 | 14.654 | 16.091 | 1.00 | 22.88 | C |
| ATOM | 3293 | CG | PHE | A | 215 | 29.271 | 15.093 | 16.179 | 1.00 | 28.57 | C |
| ATOM | 3294 | CD1 | PHE | A | 215 | 30.313 | 14.178 | 16.022 | 1.00 | 33.54 | C |
| ATOM | 3296 | CE1 | PHE | A | 215 | 31.653 | 14.548 | 16.234 | 1.00 | 33.20 | C |
| ATOM | 3298 | CZ | PHE | A | 215 | 31.988 | 15.877 | 16.490 | 1.00 | 21.98 | C |
| ATOM | 3300 | CE2 | PHE | A | 215 | 30.958 | 16.814 | 16.617 | 1.00 | 39.96 | C |
| ATOM | 3302 | CD2 | PHE | A | 215 | 29.598 | 16.414 | 16.451 | 1.00 | 21.95 | C |
| ATOM | 3304 | C | PHE | A | 215 | 26.123 | 12.870 | 15.617 | 1.00 | 27.70 | C |
| ATOM | 3305 | O | PHE | A | 215 | 25.546 | 12.591 | 16.681 | 1.00 | 35.76 | O |
| ATOM | 3306 | N | THR | A | 216 | 25.457 | 13.089 | 14.472 | 1.00 | 23.59 | N |
| ATOM | 3308 | CA | THR | A | 216 | 23.997 | 13.015 | 14.416 | 1.00 | 38.05 | C |
| ATOM | 3310 | CB | THR | A | 216 | 23.410 | 13.826 | 13.264 | 1.00 | 34.47 | C |
| ATOM | 3312 | OG1 | THR | A | 216 | 24.069 | 13.504 | 12.046 | 1.00 | 36.06 | O |
| ATOM | 3314 | CG2 | THR | A | 216 | 23.599 | 15.320 | 13.509 | 1.00 | 25.01 | C |
| ATOM | 3318 | C | THR | A | 216 | 23.478 | 11.586 | 14.361 | 1.00 | 33.99 | C |
| ATOM | 3319 | O | THR | A | 216 | 22.273 | 11.342 | 14.395 | 1.00 | 36.43 | O |
| ATOM | 3320 | N | LYS | A | 217 | 24.398 | 10.634 | 14.377 | 1.00 | 32.06 | N |
| ATOM | 3322 | CA | LYS | A | 217 | 24.018 | 9.232 | 14.508 | 1.00 | 25.83 | C |
| ATOM | 3324 | CB | LYS | A | 217 | 25.259 | 8.377 | 14.234 | 1.00 | 36.18 | C |
| ATOM | 3327 | CG | LYS | A | 217 | 25.802 | 8.512 | 12.791 | 1.00 | 44.93 | C |
| ATOM | 3330 | CD | LYS | A | 217 | 25.007 | 7.668 | 11.797 | 1.00 | 58.61 | C |
| ATOM | 3333 | CE | LYS | A | 217 | 24.568 | 8.467 | 10.597 | 1.00 | 69.99 | C |
| ATOM | 3336 | NZ | LYS | A | 217 | 25.754 | 8.927 | 9.816 | 1.00 | 79.66 | N |
| ATOM | 3340 | C | LYS | A | 217 | 23.421 | 8.926 | 15.900 | 1.00 | 32.24 | C |
| ATOM | 3341 | O | LYS | A | 217 | 22.842 | 7.856 | 16.115 | 1.00 | 30.32 | O |
| ATOM | 3342 | N | ALA | A | 218 | 23.640 | 9.843 | 16.846 | 1.00 | 28.34 | N |
| ATOM | 3344 | CA | ALA | A | 218 | 23.085 | 9.779 | 18.186 | 1.00 | 29.40 | C |
| ATOM | 3346 | CB | ALA | A | 218 | 23.922 | 10.629 | 19.137 | 1.00 | 25.19 | C |
| ATOM | 3350 | C | ALA | A | 218 | 21.641 | 10.277 | 18.225 | 1.00 | 27.31 | C |
| ATOM | 3351 | O | ALA | A | 218 | 21.033 | 10.315 | 19.281 | 1.00 | 34.87 | O |
| ATOM | 3352 | N | THR | A | 219 | 21.132 | 10.731 | 17.086 | 1.00 | 30.45 | N |
| ATOM | 3354 | CA | THR | A | 219 | 19.778 | 11.233 | 17.010 | 1.00 | 36.87 | C |
| ATOM | 3356 | CB | THR | A | 219 | 19.447 | 11.545 | 15.550 | 1.00 | 34.98 | C |
| ATOM | 3358 | OG1 | THR | A | 219 | 20.261 | 12.650 | 15.146 | 1.00 | 30.60 | O |
| ATOM | 3360 | CG2 | THR | A | 219 | 18.006 | 12.048 | 15.405 | 1.00 | 34.67 | C |
| ATOM | 3364 | C | THR | A | 219 | 18.713 | 10.341 | 17.697 | 1.00 | 26.82 | C |
| ATOM | 3365 | O | THR | A | 219 | 17.871 | 10.846 | 18.390 | 1.00 | 28.97 | O |
| ATOM | 3366 | N | PRO | A | 220 | 18.732 | 9.044 | 17.463 | 1.00 | 30.70 | N |
| ATOM | 3367 | CA | PRO | A | 220 | 17.774 | 8.133 | 18.094 | 1.00 | 29.40 | C |
| ATOM | 3369 | CB | PRO | A | 220 | 18.094 | 6.760 | 17.457 | 1.00 | 25.84 | C |
| ATOM | 3372 | CG | PRO | A | 220 | 18.829 | 7.079 | 16.203 | 1.00 | 34.75 | C |
| ATOM | 3375 | CD | PRO | A | 220 | 19.621 | 8.359 | 16.492 | 1.00 | 28.60 | C |
| ATOM | 3378 | C | PRO | A | 220 | 17.806 | 8.061 | 19.631 | 1.00 | 35.43 | C |
| ATOM | 3379 | O | PRO | A | 220 | 16.804 | 7.658 | 20.209 | 1.00 | 39.70 | O |
| ATOM | 3380 | N | LEU | A | 221 | 18.857 | 8.550 | 20.276 | 1.00 | 35.15 | N |
| ATOM | 3382 | CA | LEU | A | 221 | 18.964 | 8.537 | 21.729 | 1.00 | 32.03 | C |
| ATOM | 3384 | CB | LEU | A | 221 | 20.429 | 8.680 | 22.164 | 1.00 | 30.75 | C |
| ATOM | 3387 | CG | LEU | A | 221 | 21.399 | 7.539 | 21.848 | 1.00 | 39.71 | C |
| ATOM | 3389 | CD1 | LEU | A | 221 | 22.889 | 8.002 | 22.038 | 1.00 | 32.91 | C |
| ATOM | 3393 | CD2 | LEU | A | 221 | 21.064 | 6.306 | 22.699 | 1.00 | 34.38 | C |
| ATOM | 3397 | C | LEU | A | 221 | 18.218 | 9.627 | 22.470 | 1.00 | 35.50 | C |
| ATOM | 3398 | O | LEU | A | 221 | 17.992 | 9.538 | 23.673 | 1.00 | 37.86 | O |
| ATOM | 3399 | N | SER | A | 222 | 17.981 | 10.738 | 21.798 | 1.00 | 39.63 | N |
| ATOM | 3401 | CA | SER | A | 222 | 17.303 | 11.826 | 22.439 | 1.00 | 34.31 | C |
| ATOM | 3403 | CB | SER | A | 222 | 18.294 | 12.725 | 23.162 | 1.00 | 40.92 | C |
| ATOM | 3406 | OG | SER | A | 222 | 17.543 | 13.693 | 23.893 | 1.00 | 41.41 | O |
| ATOM | 3408 | C | SER | A | 222 | 16.463 | 12.658 | 21.465 | 1.00 | 44.31 | C |
| ATOM | 3409 | O | SER | A | 222 | 16.835 | 12.844 | 20.315 | 1.00 | 38.31 | O |
| ATOM | 3410 | N | SER | A | 223 | 15.362 | 13.214 | 21.961 | 1.00 | 38.30 | N |
| ATOM | 3412 | CA | SER | A | 223 | 14.475 | 14.016 | 21.138 | 1.00 | 41.79 | C |
| ATOM | 3414 | CB | SER | A | 223 | 13.083 | 14.193 | 21.788 | 1.00 | 41.06 | C |
| ATOM | 3417 | OG | SER | A | 223 | 12.339 | 13.003 | 21.701 | 1.00 | 64.73 | O |
| ATOM | 3419 | C | SER | A | 223 | 15.067 | 15.394 | 20.961 | 1.00 | 34.41 | C |
| ATOM | 3420 | O | SER | A | 223 | 14.637 | 16.106 | 20.068 | 1.00 | 29.31 | O |
| ATOM | 3421 | N | THR | A | 224 | 15.972 | 15.799 | 21.852 | 1.00 | 31.40 | N |
| ATOM | 3423 | CA | THR | A | 224 | 16.615 | 17.106 | 21.721 | 1.00 | 37.60 | C |
| ATOM | 3425 | CB | THR | A | 224 | 16.159 | 18.029 | 22.831 | 1.00 | 42.06 | C |
| ATOM | 3427 | OG1 | THR | A | 224 | 16.531 | 17.430 | 24.067 | 1.00 | 55.34 | O |
| ATOM | 3429 | CG2 | THR | A | 224 | 14.604 | 18.102 | 22.875 | 1.00 | 45.96 | C |
| ATOM | 3433 | C | THR | A | 224 | 18.137 | 17.051 | 21.701 | 1.00 | 30.55 | C |
| ATOM | 3434 | O | THR | A | 224 | 18.725 | 16.011 | 21.973 | 1.00 | 33.92 | O |
| ATOM | 3435 | N | VAL | A | 225 | 18.748 | 18.173 | 21.319 | 1.00 | 33.57 | N |
| ATOM | 3437 | CA | VAL | A | 225 | 20.203 | 18.304 | 21.233 | 1.00 | 28.59 | C |
| ATOM | 3439 | CB | VAL | A | 225 | 20.704 | 18.071 | 19.801 | 1.00 | 31.97 | C |
| ATOM | 3441 | CG1 | VAL | A | 225 | 20.279 | 19.193 | 18.852 | 1.00 | 33.93 | C |
| ATOM | 3445 | CG2 | VAL | A | 225 | 22.251 | 17.953 | 19.769 | 1.00 | 49.45 | C |
| ATOM | 3449 | C | VAL | A | 225 | 20.529 | 19.711 | 21.683 | 1.00 | 21.49 | C |
| ATOM | 3450 | O | VAL | A | 225 | 19.702 | 20.602 | 21.505 | 1.00 | 34.25 | O |
| ATOM | 3451 | N | THR | A | 226 | 21.634 | 19.904 | 22.403 | 1.00 | 28.15 | N |
| ATOM | 3453 | CA | THR | A | 226 | 22.044 | 21.271 | 22.754 | 1.00 | 30.56 | C |
| ATOM | 3455 | CB | THR | A | 226 | 22.208 | 21.477 | 24.287 | 1.00 | 22.78 | C |
| ATOM | 3457 | OG1 | THR | A | 226 | 23.342 | 20.721 | 24.711 | 1.00 | 50.17 | O |
| ATOM | 3459 | CG2 | THR | A | 226 | 21.013 | 20.838 | 25.063 | 1.00 | 30.95 | C |
| ATOM | 3463 | C | THR | A | 226 | 23.369 | 21.581 | 22.102 | 1.00 | 27.68 | C |
| ATOM | 3464 | O | THR | A | 226 | 24.233 | 20.709 | 22.041 | 1.00 | 37.03 | O |
| ATOM | 3465 | N | LEU | A | 227 | 23.547 | 22.847 | 21.737 | 1.00 | 33.29 | N |
| ATOM | 3467 | CA | LEU | A | 227 | 24.770 | 23.311 | 21.117 | 1.00 | 41.81 | C |
| ATOM | 3469 | CB | LEU | A | 227 | 24.477 | 23.820 | 19.698 | 1.00 | 41.29 | C |
| ATOM | 3472 | CG | LEU | A | 227 | 23.608 | 22.962 | 18.768 | 1.00 | 35.85 | C |
| ATOM | 3474 | CD1 | LEU | A | 227 | 23.293 | 23.731 | 17.512 | 1.00 | 43.87 | C |
| ATOM | 3478 | CD2 | LEU | A | 227 | 24.278 | 21.622 | 18.350 | 1.00 | 40.43 | C |
| ATOM | 3482 | C | LEU | A | 227 | 25.373 | 24.436 | 21.962 | 1.00 | 36.94 | C |
| ATOM | 3483 | O | LEU | A | 227 | 24.831 | 25.526 | 21.963 | 1.00 | 46.69 | O |
| ATOM | 3484 | N | SER | A | 228 | 26.487 | 24.169 | 22.653 | 1.00 | 32.86 | N |
| ATOM | 3486 | CA | SER | A | 228 | 27.208 | 25.213 | 23.375 | 1.00 | 34.91 | C |
| ATOM | 3488 | CB | SER | A | 228 | 27.581 | 24.785 | 24.778 | 1.00 | 35.14 | C |
| ATOM | 3491 | OG | SER | A | 228 | 26.592 | 23.988 | 25.382 | 1.00 | 34.75 | O |
| ATOM | 3493 | C | SER | A | 228 | 28.493 | 25.615 | 22.640 | 1.00 | 40.19 | C |
| ATOM | 3494 | O | SER | A | 228 | 29.307 | 24.782 | 22.241 | 1.00 | 37.39 | O |
| ATOM | 3495 | N | MET | A | 229 | 28.664 | 26.923 | 22.485 | 1.00 | 40.59 | N |
| ATOM | 3497 | CA | MET | A | 229 | 29.608 | 27.472 | 21.531 | 1.00 | 33.22 | C |
| ATOM | 3499 | CB | MET | A | 229 | 28.865 | 27.952 | 20.302 | 1.00 | 36.47 | C |
| ATOM | 3502 | CG | MET | A | 229 | 28.744 | 26.977 | 19.181 | 1.00 | 46.24 | C |
| ATOM | 3505 | SD | MET | A | 229 | 27.750 | 27.775 | 17.924 | 1.00 | 55.69 | S |
| ATOM | 3506 | CE | MET | A | 229 | 26.168 | 27.779 | 18.742 | 1.00 | 54.38 | C |
| ATOM | 3510 | C | MET | A | 229 | 30.238 | 28.719 | 22.119 | 1.00 | 43.94 | C |
| ATOM | 3511 | O | MET | A | 229 | 29.589 | 29.445 | 22.873 | 1.00 | 40.30 | O |
| ATOM | 3512 | N | SER | A | 230 | 31.486 | 28.968 | 21.705 | 1.00 | 47.17 | N |
| ATOM | 3514 | CA | SER | A | 230 | 32.254 | 30.143 | 22.079 | 1.00 | 47.23 | C |
| ATOM | 3516 | CB | SER | A | 230 | 32.769 | 30.001 | 23.503 | 1.00 | 52.16 | C |
| ATOM | 3519 | OG | SER | A | 230 | 32.244 | 31.040 | 24.301 | 1.00 | 56.24 | O |
| ATOM | 3521 | C | SER | A | 230 | 33.423 | 30.335 | 21.108 | 1.00 | 46.03 | C |
| ATOM | 3522 | O | SER | A | 230 | 33.821 | 29.409 | 20.402 | 1.00 | 42.34 | O |
| ATOM | 3523 | N | ALA | A | 231 | 33.903 | 31.570 | 21.016 | 1.00 | 48.06 | N |
| ATOM | 3525 | CA | ALA | A | 231 | 35.071 | 31.904 | 20.194 | 1.00 | 47.33 | C |
| ATOM | 3527 | CB | ALA | A | 231 | 35.462 | 33.347 | 20.416 | 1.00 | 48.65 | C |
| ATOM | 3531 | C | ALA | A | 231 | 36.227 | 31.030 | 20.600 | 1.00 | 41.43 | C |
| ATOM | 3532 | O | ALA | A | 231 | 36.591 | 30.995 | 21.762 | 1.00 | 48.70 | O |
| ATOM | 3533 | N | ASP | A | 232 | 36.762 | 30.280 | 19.654 | 1.00 | 53.04 | N |
| ATOM | 3535 | CA | ASP | A | 232 | 38.055 | 29.627 | 19.827 | 1.00 | 63.95 | C |
| ATOM | 3537 | CB | ASP | A | 232 | 39.068 | 30.595 | 20.481 | 1.00 | 71.07 | C |
| ATOM | 3540 | CG | ASP | A | 232 | 39.173 | 31.931 | 19.737 | 1.00 | 80.55 | C |
| ATOM | 3541 | OD1 | ASP | A | 232 | 39.166 | 33.006 | 20.390 | 1.00 | 79.96 | O |
| ATOM | 3542 | OD2 | ASP | A | 232 | 39.260 | 31.991 | 18.489 | 1.00 | 87.40 | O |
| ATOM | 3543 | C | ASP | A | 232 | 37.989 | 28.307 | 20.596 | 1.00 | 61.85 | C |
| ATOM | 3544 | O | ASP | A | 232 | 38.876 | 27.476 | 20.437 | 1.00 | 66.64 | O |
| ATOM | 3545 | N | VAL | A | 233 | 36.959 | 28.109 | 21.421 | 1.00 | 63.30 | N |
| ATOM | 3547 | CA | VAL | A | 233 | 36.751 | 26.820 | 22.095 | 1.00 | 57.60 | C |
| ATOM | 3549 | CB | VAL | A | 233 | 36.140 | 27.005 | 23.509 | 1.00 | 63.37 | C |
| ATOM | 3551 | CG1 | VAL | A | 233 | 36.788 | 28.211 | 24.203 | 1.00 | 69.38 | C |
| ATOM | 3555 | CG2 | VAL | A | 233 | 34.620 | 27.171 | 23.463 | 1.00 | 70.54 | C |
| ATOM | 3559 | C | VAL | A | 233 | 35.937 | 25.829 | 21.250 | 1.00 | 48.23 | C |
| ATOM | 3560 | O | VAL | A | 233 | 35.229 | 26.208 | 20.329 | 1.00 | 46.57 | O |
| ATOM | 3561 | N | PRO | A | 234 | 36.111 | 24.543 | 21.506 | 1.00 | 41.25 | N |
| ATOM | 3562 | CA | PRO | A | 234 | 35.305 | 23.518 | 20.835 | 1.00 | 39.55 | C |
| ATOM | 3564 | CB | PRO | A | 234 | 35.875 | 22.198 | 21.360 | 1.00 | 35.42 | C |
| ATOM | 3567 | CG | PRO | A | 234 | 37.278 | 22.587 | 21.941 | 1.00 | 49.59 | C |
| ATOM | 3570 | CD | PRO | A | 234 | 37.065 | 23.985 | 22.487 | 1.00 | 39.89 | C |
| ATOM | 3573 | C | PRO | A | 234 | 33.800 | 23.607 | 21.142 | 1.00 | 46.21 | C |
| ATOM | 3574 | O | PRO | A | 234 | 33.388 | 23.860 | 22.278 | 1.00 | 34.93 | O |
| ATOM | 3575 | N | LEU | A | 235 | 33.005 | 23.461 | 20.085 | 1.00 | 44.62 | N |
| ATOM | 3577 | CA | LEU | A | 235 | 31.568 | 23.248 | 20.182 | 1.00 | 38.40 | C |
| ATOM | 3579 | CB | LEU | A | 235 | 31.019 | 23.063 | 18.766 | 1.00 | 38.21 | C |
| ATOM | 3582 | CG | LEU | A | 235 | 29.590 | 22.566 | 18.530 | 1.00 | 28.19 | C |
| ATOM | 3584 | CD1 | LEU | A | 235 | 28.543 | 23.441 | 19.138 | 1.00 | 33.76 | C |
| ATOM | 3588 | CD2 | LEU | A | 235 | 29.350 | 22.385 | 17.011 | 1.00 | 27.63 | C |
| ATOM | 3592 | C | LEU | A | 235 | 31.316 | 21.997 | 20.965 | 1.00 | 35.16 | C |
| ATOM | 3593 | O | LEU | A | 235 | 31.995 | 20.991 | 20.756 | 1.00 | 38.51 | O |
| ATOM | 3594 | N | VAL | A | 236 | 30.306 | 22.022 | 21.822 | 1.00 | 36.34 | N |
| ATOM | 3596 | CA | VAL | A | 236 | 29.795 | 20.774 | 22.391 | 1.00 | 38.96 | C |
| ATOM | 3598 | CB | VAL | A | 236 | 29.672 | 20.884 | 23.925 | 1.00 | 43.43 | C |
| ATOM | 3600 | CG1 | VAL | A | 236 | 29.539 | 19.526 | 24.543 | 1.00 | 36.47 | C |
| ATOM | 3604 | CG2 | VAL | A | 236 | 30.918 | 21.559 | 24.493 | 1.00 | 49.45 | C |
| ATOM | 3608 | C | VAL | A | 236 | 28.431 | 20.444 | 21.797 | 1.00 | 36.87 | C |
| ATOM | 3609 | O | VAL | A | 236 | 27.590 | 21.330 | 21.690 | 1.00 | 41.84 | O |
| ATOM | 3610 | N | VAL | A | 237 | 28.224 | 19.174 | 21.437 | 1.00 | 37.31 | N |
| ATOM | 3612 | CA | VAL | A | 237 | 26.941 | 18.682 | 20.978 | 1.00 | 30.73 | C |
| ATOM | 3614 | CB | VAL | A | 237 | 27.076 | 18.143 | 19.549 | 1.00 | 34.10 | C |
| ATOM | 3616 | CG1 | VAL | A | 237 | 25.726 | 17.707 | 19.017 | 1.00 | 27.15 | C |
| ATOM | 3620 | CG2 | VAL | A | 237 | 27.680 | 19.200 | 18.650 | 1.00 | 23.38 | C |
| ATOM | 3624 | C | VAL | A | 237 | 26.422 | 17.608 | 21.943 | 1.00 | 32.98 | C |
| ATOM | 3625 | O | VAL | A | 237 | 26.916 | 16.484 | 21.980 | 1.00 | 42.01 | O |
| ATOM | 3626 | N | GLU | A | 238 | 25.442 | 17.971 | 22.766 | 1.00 | 34.16 | N |
| ATOM | 3628 | CA | GLU | A | 238 | 25.037 | 17.080 | 23.846 | 1.00 | 36.65 | C |
| ATOM | 3630 | CB | GLU | A | 238 | 24.850 | 17.836 | 25.158 | 1.00 | 40.69 | C |
| ATOM | 3633 | CG | GLU | A | 238 | 24.547 | 16.915 | 26.323 | 1.00 | 45.15 | C |
| ATOM | 3636 | CD | GLU | A | 238 | 24.746 | 17.613 | 27.651 | 1.00 | 56.86 | C |
| ATOM | 3637 | OE1 | GLU | A | 238 | 24.702 | 18.859 | 27.634 | 1.00 | 49.86 | O |
| ATOM | 3638 | OE2 | GLU | A | 238 | 24.968 | 16.922 | 28.675 | 1.00 | 72.52 | O |
| ATOM | 3639 | C | GLU | A | 238 | 23.751 | 16.368 | 23.546 | 1.00 | 25.80 | C |
| ATOM | 3640 | O | GLU | A | 238 | 22.830 | 17.020 | 23.087 | 1.00 | 29.39 | O |
| ATOM | 3641 | N | TYR | A | 239 | 23.700 | 15.068 | 23.868 | 1.00 | 30.34 | N |
| ATOM | 3643 | CA | TYR | A | 239 | 22.523 | 14.226 | 23.742 | 1.00 | 26.45 | C |
| ATOM | 3645 | CB | TYR | A | 239 | 22.754 | 13.153 | 22.681 | 1.00 | 29.75 | C |
| ATOM | 3648 | CG | TYR | A | 239 | 22.815 | 13.667 | 21.249 | 1.00 | 29.21 | C |
| ATOM | 3649 | CD1 | TYR | A | 239 | 24.032 | 14.017 | 20.661 | 1.00 | 30.48 | C |
| ATOM | 3651 | CE1 | TYR | A | 239 | 24.100 | 14.421 | 19.355 | 1.00 | 27.34 | C |
| ATOM | 3653 | CZ | TYR | A | 239 | 22.924 | 14.588 | 18.617 | 1.00 | 25.50 | C |
| ATOM | 3654 | OH | TYR | A | 239 | 23.021 | 15.043 | 17.317 | 1.00 | 27.08 | O |
| ATOM | 3656 | CE2 | TYR | A | 239 | 21.702 | 14.307 | 19.179 | 1.00 | 28.58 | C |
| ATOM | 3658 | CD2 | TYR | A | 239 | 21.660 | 13.818 | 20.492 | 1.00 | 33.37 | C |
| ATOM | 3660 | C | TYR | A | 239 | 22.334 | 13.532 | 25.088 | 1.00 | 36.66 | C |
| ATOM | 3661 | O | TYR | A | 239 | 23.256 | 12.901 | 25.583 | 1.00 | 34.22 | O |
| ATOM | 3662 | N | LYS | A | 240 | 21.115 | 13.580 | 25.615 | 1.00 | 38.49 | N |
| ATOM | 3664 | CA | LYS | A | 240 | 20.767 | 13.004 | 26.898 | 1.00 | 45.06 | C |
| ATOM | 3666 | CB | LYS | A | 240 | 19.513 | 13.716 | 27.415 | 1.00 | 51.28 | C |
| ATOM | 3669 | CG | LYS | A | 240 | 19.505 | 14.066 | 28.890 | 1.00 | 62.06 | C |
| ATOM | 3672 | CD | LYS | A | 240 | 20.222 | 15.402 | 29.139 | 1.00 | 81.02 | C |
| ATOM | 3675 | CE | LYS | A | 240 | 19.254 | 16.561 | 29.378 | 1.00 | 83.65 | C |
| ATOM | 3678 | NZ | LYS | A | 240 | 19.744 | 17.826 | 28.733 | 1.00 | 81.16 | N |
| ATOM | 3682 | C | LYS | A | 240 | 20.390 | 11.565 | 26.654 | 1.00 | 43.14 | C |
| ATOM | 3683 | O | LYS | A | 240 | 19.641 | 11.307 | 25.725 | 1.00 | 51.37 | O |
| ATOM | 3684 | N | ILE | A | 241 | 20.792 | 10.648 | 27.529 | 1.00 | 35.64 | N |
| ATOM | 3686 | CA | ILE | A | 241 | 20.478 | 9.249 | 27.296 | 1.00 | 38.93 | C |
| ATOM | 3688 | CB | ILE | A | 241 | 21.747 | 8.373 | 27.380 | 1.00 | 37.98 | C |
| ATOM | 3690 | CG1 | ILE | A | 241 | 22.661 | 8.672 | 26.195 | 1.00 | 36.57 | C |
| ATOM | 3693 | CD1 | ILE | A | 241 | 24.054 | 8.097 | 26.340 | 1.00 | 43.00 | C |
| ATOM | 3697 | CG2 | ILE | A | 241 | 21.367 | 6.918 | 27.441 | 1.00 | 44.02 | C |
| ATOM | 3701 | C | ILE | A | 241 | 19.444 | 8.840 | 28.321 | 1.00 | 47.15 | C |
| ATOM | 3702 | O | ILE | A | 241 | 19.769 | 8.256 | 29.341 | 1.00 | 49.99 | O |
| ATOM | 3703 | N | ALA | A | 242 | 18.198 | 9.236 | 28.084 | 1.00 | 56.91 | N |
| ATOM | 3705 | CA | ALA | A | 242 | 17.060 | 8.731 | 28.853 | 1.00 | 56.98 | C |
| ATOM | 3707 | CB | ALA | A | 242 | 16.516 | 7.451 | 28.170 | 1.00 | 49.91 | C |
| ATOM | 3711 | C | ALA | A | 242 | 17.410 | 8.413 | 30.296 | 1.00 | 56.22 | C |
| ATOM | 3712 | O | ALA | A | 242 | 17.619 | 7.254 | 30.626 | 1.00 | 71.25 | O |
| ATOM | 3713 | N | ASP | A | 243 | 17.488 | 9.384 | 31.183 | 1.00 | 56.25 | N |
| ATOM | 3715 | CA | ASP | A | 243 | 17.748 | 9.033 | 32.599 | 1.00 | 65.27 | C |
| ATOM | 3717 | CB | ASP | A | 243 | 16.429 | 8.648 | 33.296 | 1.00 | 69.43 | C |
| ATOM | 3720 | CG | ASP | A | 243 | 15.396 | 9.780 | 33.288 | 1.00 | 78.32 | C |
| ATOM | 3721 | OD1 | ASP | A | 243 | 15.720 | 10.915 | 33.714 | 1.00 | 69.23 | O |
| ATOM | 3722 | OD2 | ASP | A | 243 | 14.231 | 9.627 | 32.852 | 1.00 | 91.76 | O |
| ATOM | 3723 | C | ASP | A | 243 | 18.799 | 7.931 | 32.932 | 1.00 | 64.14 | C |
| ATOM | 3724 | O | ASP | A | 243 | 18.607 | 7.165 | 33.878 | 1.00 | 72.92 | O |
| ATOM | 3725 | N | MET | A | 244 | 19.909 | 7.849 | 32.195 | 1.00 | 64.39 | N |
| ATOM | 3727 | CA | MET | A | 244 | 21.089 | 7.099 | 32.637 | 1.00 | 53.21 | C |
| ATOM | 3729 | CB | MET | A | 244 | 21.394 | 5.856 | 31.779 | 1.00 | 54.66 | C |
| ATOM | 3732 | CG | MET | A | 244 | 20.285 | 5.226 | 30.988 | 1.00 | 43.21 | C |
| ATOM | 3735 | SD | MET | A | 244 | 20.526 | 3.455 | 30.941 | 1.00 | 71.81 | S |
| ATOM | 3736 | CE | MET | A | 244 | 22.071 | 3.295 | 30.062 | 1.00 | 70.41 | C |
| ATOM | 3740 | C | MET | A | 244 | 22.338 | 7.945 | 32.498 | 1.00 | 49.74 | C |
| ATOM | 3741 | O | MET | A | 244 | 23.355 | 7.630 | 33.115 | 1.00 | 50.18 | O |
| ATOM | 3742 | N | GLY | A | 245 | 22.327 | 8.887 | 31.561 | 1.00 | 40.35 | N |
| ATOM | 3744 | CA | GLY | A | 245 | 23.430 | 9.811 | 31.424 | 1.00 | 31.92 | C |
| ATOM | 3747 | C | GLY | A | 245 | 23.432 | 10.696 | 30.209 | 1.00 | 34.29 | C |
| ATOM | 3748 | O | GLY | A | 245 | 22.411 | 11.316 | 29.930 | 1.00 | 33.30 | O |
| ATOM | 3749 | N | HIS | A | 246 | 24.584 | 10.807 | 29.531 | 1.00 | 39.14 | N |
| ATOM | 3751 | CA | HIS | A | 246 | 24.751 | 11.752 | 28.426 | 1.00 | 34.86 | C |
| ATOM | 3753 | CB | HIS | A | 246 | 25.114 | 13.153 | 28.949 | 1.00 | 43.01 | C |
| ATOM | 3756 | CG | HIS | A | 246 | 26.517 | 13.285 | 29.480 | 1.00 | 45.86 | C |
| ATOM | 3757 | ND1 | HIS | A | 246 | 26.854 | 14.218 | 30.439 | 1.00 | 72.89 | N |
| ATOM | 3759 | CE1 | HIS | A | 246 | 28.141 | 14.117 | 30.727 | 1.00 | 66.28 | C |
| ATOM | 3761 | NE2 | HIS | A | 246 | 20.652 | 13.135 | 30.008 | 1.00 | 52.61 | N |
| ATOM | 3763 | CD2 | HIS | A | 246 | 27.658 | 12.600 | 29.215 | 1.00 | 69.45 | C |
| ATOM | 3765 | C | HIS | A | 246 | 25.797 | 11.316 | 27.404 | 1.00 | 39.42 | C |
| ATOM | 3766 | O | HIS | A | 246 | 26.668 | 10.504 | 27.707 | 1.00 | 34.06 | O |
| ATOM | 3767 | N | LEU | A | 247 | 25.759 | 11.946 | 26.227 | 1.00 | 41.17 | N |
| ATOM | 3769 | CA | LEU | A | 247 | 26.754 | 11.761 | 25.168 | 1.00 | 35.34 | C |
| ATOM | 3771 | CB | LEU | A | 247 | 26.170 | 10.831 | 24.094 | 1.00 | 35.71 | C |
| ATOM | 3774 | CG | LEU | A | 247 | 27.038 | 10.351 | 22.923 | 1.00 | 40.04 | C |
| ATOM | 3776 | CD1 | LEU | A | 247 | 28.476 | 10.096 | 23.306 | 1.00 | 49.02 | C |
| ATOM | 3780 | CD2 | LEU | A | 247 | 26.431 | 9.103 | 22.353 | 1.00 | 45.24 | C |
| ATOM | 3784 | C | LEU | A | 247 | 27.165 | 13.127 | 24.586 | 1.00 | 39.65 | C |
| ATOM | 3785 | O | LEU | A | 247 | 26.328 | 13.879 | 24.077 | 1.00 | 52.95 | O |
| ATOM | 3786 | N | LYS | A | 248 | 28.447 | 13.471 | 24.681 | 1.00 | 38.20 | N |
| ATOM | 3788 | CA | LYS | A | 248 | 28.948 | 14.775 | 24.243 | 1.00 | 34.17 | C |
| ATOM | 3790 | CB | LYS | A | 248 | 29.563 | 15.557 | 25.401 | 1.00 | 36.17 | C |
| ATOM | 3793 | CG | LYS | A | 248 | 28.516 | 15.968 | 26.463 | 1.00 | 34.80 | C |
| ATOM | 3796 | CD | LYS | A | 248 | 29.138 | 16.728 | 27.646 | 1.00 | 40.13 | C |
| ATOM | 3799 | CE | LYS | A | 248 | 28.158 | 16.770 | 28.834 | 1.00 | 56.87 | C |
| ATOM | 3802 | NZ | LYS | A | 248 | 28.383 | 17.866 | 29.841 | 1.00 | 53.90 | N |
| ATOM | 3806 | C | LYS | A | 248 | 29.996 | 14.603 | 23.158 | 1.00 | 37.20 | C |
| ATOM | 3807 | O | LYS | A | 248 | 30.907 | 13.771 | 23.264 | 1.00 | 33.24 | O |
| ATOM | 3808 | N | TYR | A | 249 | 29.847 | 15.389 | 22.103 | 1.00 | 35.07 | N |
| ATOM | 3810 | CA | TYR | A | 249 | 30.788 | 15.389 | 21.020 | 1.00 | 27.67 | C |
| ATOM | 3812 | CB | TYR | A | 249 | 30.147 | 15.071 | 19.683 | 1.00 | 32.35 | C |
| ATOM | 3815 | CG | TYR | A | 249 | 29.479 | 13.730 | 19.521 | 1.00 | 29.45 | C |
| ATOM | 3816 | CD1 | TYR | A | 249 | 30.156 | 12.678 | 18.940 | 1.00 | 28.85 | C |
| ATOM | 3818 | CE1 | TYR | A | 249 | 29.519 | 11.474 | 18.690 | 1.00 | 25.84 | C |
| ATOM | 3820 | CZ | TYR | A | 249 | 28.171 | 11.341 | 19.039 | 1.00 | 37.04 | C |
| ATOM | 3821 | OH | TYR | A | 249 | 27.491 | 10.167 | 18.845 | 1.00 | 30.08 | O |
| ATOM | 3823 | CE2 | TYR | A | 249 | 27.486 | 12.377 | 19.605 | 1.00 | 22.23 | C |
| ATOM | 3825 | CD2 | TYR | A | 249 | 28.128 | 13.553 | 19.863 | 1.00 | 31.99 | C |
| ATOM | 3827 | C | TYR | A | 249 | 31.358 | 16.792 | 20.998 | 1.00 | 35.08 | C |
| ATOM | 3828 | O | TYR | A | 249 | 30.663 | 17.759 | 21.310 | 1.00 | 36.91 | O |
| ATOM | 3829 | N | TYR | A | 250 | 32.667 | 16.874 | 20.771 | 1.00 | 37.75 | N |
| ATOM | 3831 | CA | TYR | A | 250 | 33.388 | 18.133 | 20.885 | 1.00 | 32.67 | C |
| ATOM | 3833 | CB | TYR | A | 250 | 34.416 | 18.087 | 22.015 | 1.00 | 47.18 | C |
| ATOM | 3836 | CG | TYR | A | 250 | 33.871 | 18.090 | 23.432 | 1.00 | 44.49 | C |
| ATOM | 3837 | CD1 | TYR | A | 250 | 33.557 | 16.903 | 24.062 | 1.00 | 53.76 | C |
| ATOM | 3839 | CE1 | TYR | A | 250 | 33.088 | 16.878 | 25.356 | 1.00 | 39.65 | C |
| ATOM | 3841 | CZ | TYR | A | 250 | 32.957 | 18.046 | 26.046 | 1.00 | 55.29 | C |
| ATOM | 3842 | OH | TYR | A | 250 | 32.471 | 17.947 | 27.330 | 1.00 | 63.64 | O |
| ATOM | 3844 | CE2 | TYR | A | 250 | 33.263 | 19.260 | 25.448 | 1.00 | 56.99 | C |
| ATOM | 3846 | CD2 | TYR | A | 250 | 33.725 | 19.274 | 24.148 | 1.00 | 48.95 | C |
| ATOM | 3848 | C | TYR | A | 250 | 34.134 | 18.276 | 19.590 | 1.00 | 33.14 | C |
| ATOM | 3049 | O | TYR | A | 250 | 34.793 | 17.327 | 19.110 | 1.00 | 30.05 | O |
| ATOM | 3850 | N | LEU | A | 251 | 34.007 | 19.472 | 19.031 | 1.00 | 35.87 | N |
| ATOM | 3852 | CA | LEU | A | 251 | 34.598 | 19.803 | 17.743 | 1.00 | 38.90 | C |
| ATOM | 3854 | CB | LEU | A | 251 | 33.493 | 19.763 | 16.701 | 1.00 | 36.11 | C |
| ATOM | 3857 | CG | LEU | A | 251 | 33.894 | 19.940 | 15.250 | 1.00 | 37.24 | C |
| ATOM | 3859 | CD1 | LEU | A | 251 | 34.887 | 18.856 | 14.929 | 1.00 | 35.32 | C |
| ATOM | 3863 | CD2 | LEU | A | 251 | 32.622 | 19.835 | 14.392 | 1.00 | 31.87 | C |
| ATOM | 3867 | C | LEU | A | 251 | 35.217 | 21.203 | 17.778 | 1.00 | 43.89 | C |
| ATOM | 3868 | O | LEU | A | 251 | 34.532 | 22.182 | 18.104 | 1.00 | 43.50 | O |
| ATOM | 3869 | N | ALA | A | 252 | 36.497 | 21.306 | 17.412 | 1.00 | 46.34 | N |
| ATOM | 3871 | CA | ALA | A | 252 | 37.142 | 22.616 | 17.276 | 1.00 | 45.02 | C |
| ATOM | 3873 | CB | ALA | A | 252 | 38.683 | 22.511 | 17.429 | 1.00 | 51.65 | C |
| ATOM | 3877 | C | ALA | A | 252 | 36.796 | 23.262 | 10.941 | 1.00 | 43.02 | C |
| ATOM | 3878 | O | ALA | A | 202 | 36.684 | 22.597 | 14.890 | 1.00 | 37.75 | O |
| ATOM | 3879 | N | PRO | A | 253 | 36.685 | 24.577 | 10.982 | 1.00 | 39.10 | N |
| ATOM | 3880 | CA | PRO | A | 253 | 36.404 | 25.345 | 14.784 | 1.00 | 54.99 | C |
| ATOM | 3882 | CB | PRO | A | 253 | 36.004 | 26.714 | 15.339 | 1.00 | 49.33 | C |
| ATOM | 3885 | CG | PRO | A | 253 | 35.864 | 26.523 | 16.811 | 1.00 | 56.82 | C |
| ATOM | 3888 | CD | PRO | A | 253 | 36.877 | 25.464 | 17.141 | 1.00 | 54.55 | C |
| ATOM | 3891 | C | PRO | A | 253 | 37.651 | 25.425 | 13.923 | 1.00 | 58.55 | C |
| ATOM | 3892 | O | PRO | A | 253 | 38.749 | 25.249 | 14.440 | 1.00 | 68.48 | O |
| ATOM | 3893 | N | LYS | A | 254 | 37.463 | 25.573 | 12.618 | 1.00 | 58.27 | N |
| ATOM | 3895 | CA | LYS | A | 254 | 38.451 | 26.202 | 11.755 | 1.00 | 62.99 | C |
| ATOM | 3897 | CB | LYS | A | 254 | 37.910 | 26.219 | 10.326 | 1.00 | 65.17 | C |
| ATOM | 3900 | CG | LYS | A | 254 | 37.791 | 24.797 | 9.742 | 1.00 | 73.45 | C |
| ATOM | 3903 | CD | LYS | A | 254 | 36.651 | 24.662 | 8.736 | 1.00 | 71.78 | C |
| ATOM | 3906 | CE | LYS | A | 254 | 36.742 | 23.364 | 7.936 | 1.00 | 70.97 | C |
| ATOM | 3909 | NZ | LYS | A | 254 | 37.598 | 22.334 | 8.605 | 1.00 | 65.99 | N |
| ATOM | 3913 | C | LYS | A | 254 | 38.810 | 27.618 | 12.224 | 1.00 | 61.20 | C |
| ATOM | 3914 | O | LYS | A | 254 | 37.996 | 28.519 | 12.141 | 1.00 | 61.72 | O |
| ATOM | 3915 | N | ILE | A | 255 | 40.027 | 27.812 | 12.725 | 1.00 | 67.70 | N |
| ATOM | 3917 | CA | ILE | A | 255 | 40.478 | 29.127 | 13.188 | 1.00 | 75.08 | C |
| ATOM | 3919 | CB | ILE | A | 255 | 40.331 | 30.177 | 12.071 | 1.00 | 74.53 | C |
| ATOM | 3921 | CG1 | ILE | A | 255 | 41.198 | 29.814 | 10.872 | 1.00 | 65.91 | C |
| ATOM | 3924 | CD1 | ILE | A | 255 | 41.355 | 30.962 | 9.874 | 1.00 | 85.68 | C |
| ATOM | 3928 | CG2 | ILE | A | 255 | 40.622 | 31.581 | 12.608 | 1.00 | 83.09 | C |
| ATOM | 3932 | C | ILE | A | 255 | 39.670 | 29.581 | 14.410 | 1.00 | 79.35 | C |
| ATOM | 3933 | O | ILE | A | 255 | 40.222 | 30.105 | 15.383 | 1.00 | 84.18 | O |
| ATOM | 3934 | N | MET | B | 1 | 2.332 | −32.690 | −24.060 | 1.00 | 38.07 | N |
| ATOM | 3936 | CA | MET | B | 1 | 2.259 | −31.348 | −23.405 | 1.00 | 44.88 | C |
| ATOM | 3938 | CB | MET | B | 1 | 0.880 | −30.748 | −23.656 | 1.00 | 47.82 | C |
| ATOM | 3941 | CG | MET | B | 1 | 0.918 | −29.456 | −24.426 | 1.00 | 62.01 | C |
| ATOM | 3944 | SD | MET | B | 1 | 0.372 | −28.041 | −23.436 | 1.00 | 77.14 | S |
| ATOM | 3945 | CE | MET | B | 1 | −1.223 | −28.643 | −22.748 | 1.00 | 62.87 | C |
| ATOM | 3949 | C | MET | B | 1 | 2.438 | −31.399 | −21.891 | 1.00 | 44.10 | C |
| ATOM | 3950 | O | MET | B | 1 | 1.875 | −32.275 | −21.238 | 1.00 | 47.18 | O |
| ATOM | 3953 | N | PHE | B | 2 | 3.108 | −30.381 | −21.347 | 1.00 | 38.72 | N |
| ATOM | 3955 | CA | PHE | B | 2 | 3.094 | −30.068 | −19.929 | 1.00 | 36.98 | C |
| ATOM | 3957 | CB | PHE | B | 2 | 4.513 | −29.868 | −19.432 | 1.00 | 37.70 | C |
| ATOM | 3960 | CG | PHE | B | 2 | 4.590 | −29.207 | −18.094 | 1.00 | 34.57 | C |
| ATOM | 3961 | CD1 | PHE | B | 2 | 3.994 | −29.792 | −17.004 | 1.00 | 27.69 | C |
| ATOM | 3963 | CE1 | PHE | B | 2 | 4.138 | −29.240 | −15.764 | 1.00 | 27.72 | C |
| ATOM | 3965 | CZ | PHE | B | 2 | 4.810 | −28.065 | −15.617 | 1.00 | 40.46 | C |
| ATOM | 3967 | CE2 | PHE | B | 2 | 5.434 | −27.473 | −16.697 | 1.00 | 29.95 | C |
| ATOM | 3969 | CD2 | PHE | B | 2 | 5.301 | −28.026 | −17.922 | 1.00 | 34.93 | C |
| ATOM | 3971 | C | PHE | B | 2 | 2.266 | −28.829 | −19.564 | 1.00 | 37.55 | C |
| ATOM | 3972 | O | PHE | B | 2 | 2.392 | −27.812 | −20.215 | 1.00 | 35.07 | O |
| ATOM | 3973 | N | GLU | B | 3 | 1.413 | −28.933 | −18.541 | 1.00 | 30.40 | N |
| ATOM | 3975 | CA | GLU | B | 3 | 0.709 | −27.768 | −18.039 | 1.00 | 31.69 | C |
| ATOM | 3977 | CB | GLU | B | 3 | −0.530 | −27.532 | −18.892 | 1.00 | 34.01 | C |
| ATOM | 3980 | CG | GLU | B | 3 | −1.520 | −26.547 | −18.315 | 1.00 | 41.65 | C |
| ATOM | 3983 | CD | GLU | B | 3 | −2.761 | −26.411 | −19.199 | 1.00 | 61.44 | C |
| ATOM | 3984 | OE1 | GLU | B | 3 | −3.435 | −27.434 | −19.460 | 1.00 | 76.84 | O |
| ATOM | 3985 | OE2 | GLU | B | 3 | −3.066 | −25.284 | −19.640 | 1.00 | 52.52 | O |
| ATOM | 3986 | C | GLU | B | 3 | 0.353 | −27.938 | −16.583 | 1.00 | 29.65 | C |
| ATOM | 3987 | O | GLU | B | 3 | −0.218 | −28.948 | −16.183 | 1.00 | 41.88 | O |
| ATOM | 3988 | N | ALA | B | 4 | 0.618 | −26.895 | −15.810 | 1.00 | 33.84 | N |
| ATOM | 3990 | CA | ALA | B | 4 | 0.361 | −26.895 | −14.385 | 1.00 | 33.48 | C |
| ATOM | 3992 | CB | ALA | B | 4 | 1.659 | −27.175 | −13.628 | 1.00 | 33.46 | C |
| ATOM | 3996 | C | ALA | B | 4 | −0.193 | −25.514 | −14.019 | 1.00 | 38.18 | C |
| ATOM | 3997 | O | ALA | B | 4 | 0.315 | −24.507 | −14.499 | 1.00 | 40.22 | O |
| ATOM | 3998 | N | ARG | B | 5 | −1.220 | −25.470 | −13.166 | 1.00 | 38.28 | N |
| ATOM | 4000 | CA | ARG | B | 5 | −1.732 | −24.213 | −12.640 | 1.00 | 36.62 | C |
| ATOM | 4002 | CB | ARG | B | 5 | −3.139 | −23.968 | −13.205 | 1.00 | 41.44 | C |
| ATOM | 4005 | CG | ARG | B | 5 | −3.580 | −22.521 | −13.248 | 1.00 | 58.38 | C |
| ATOM | 4008 | CD | ARG | B | 5 | −4.559 | −22.208 | −14.396 | 1.00 | 69.97 | C |
| ATOM | 4011 | NE | ARG | B | 5 | −5.781 | −23.012 | −14.335 | 1.00 | 66.84 | N |
| ATOM | 4013 | CZ | ARG | B | 5 | −7.015 | −22.514 | −14.275 | 1.00 | 80.12 | C |
| ATOM | 4014 | NH1 | ARG | B | 5 | −7.235 | −21.195 | −14.282 | 1.00 | 70.18 | N |
| ATOM | 4017 | NH2 | ARG | B | 5 | −8.045 | −23.352 | −14.213 | 1.00 | 79.64 | N |
| ATOM | 4020 | C | ARG | B | 5 | −1.728 | −24.194 | −11.110 | 1.00 | 32.52 | C |
| ATOM | 4021 | O | ARG | B | 5 | −1.968 | −25.209 | −10.472 | 1.00 | 41.23 | O |
| ATOM | 4022 | N | LEU | B | 6 | −1.568 | −23.000 | −10.546 | 1.00 | 40.33 | N |
| ATOM | 4024 | CA | LEU | B | 6 | −1.325 | −22.715 | −9.122 | 1.00 | 36.66 | C |
| ATOM | 4026 | CB | LEU | B | 6 | 0.172 | −22.411 | −8.935 | 1.00 | 49.57 | C |
| ATOM | 4029 | CG | LEU | B | 6 | 0.950 | −22.955 | −7.732 | 1.00 | 59.45 | C |
| ATOM | 4031 | CD1 | LEU | B | 6 | 0.625 | −24.422 | −7.494 | 1.00 | 69.06 | C |
| ATOM | 4035 | CD2 | LEU | B | 6 | 2.451 | −22.801 | −7.926 | 1.00 | 56.80 | C |
| ATOM | 4039 | C | LEU | B | 6 | −2.082 | −21.414 | −8.845 | 1.00 | 42.56 | C |
| ATOM | 4040 | O | LEU | B | 6 | −2.111 | −20.547 | −9.726 | 1.00 | 33.95 | O |
| ATOM | 4041 | N | VAL | B | 7 | −2.748 | −21.303 | −7.690 | 1.00 | 40.45 | N |
| ATOM | 4043 | CA | VAL | B | 7 | −3.228 | −20.013 | −7.163 | 1.00 | 44.65 | C |
| ATOM | 4045 | CB | VAL | B | 7 | −4.418 | −20.204 | −6.126 | 1.00 | 45.34 | C |
| ATOM | 4047 | CG1 | VAL | B | 7 | −5.577 | −19.327 | −6.443 | 1.00 | 48.77 | C |
| ATOM | 4051 | CG2 | VAL | B | 7 | −4.862 | −21.621 | −6.021 | 1.00 | 53.50 | C |
| ATOM | 4055 | C | VAL | B | 7 | −2.078 | −19.380 | −6.347 | 1.00 | 42.62 | C |
| ATOM | 4056 | O | VAL | B | 7 | −1.924 | −18.168 | −6.290 | 1.00 | 42.74 | O |
| ATOM | 4057 | N | GLN | B | 8 | −1.319 | −20.219 | −5.644 | 1.00 | 55.71 | N |
| ATOM | 4059 | CA | GLN | B | 8 | −0.170 | −19.775 | −4.832 | 1.00 | 63.68 | C |
| ATOM | 4061 | CB | GLN | B | 8 | 0.237 | −20.881 | −3.850 | 1.00 | 64.65 | C |
| ATOM | 4064 | CG | GLN | B | 8 | −0.836 | −21.279 | −2.850 | 1.00 | 71.79 | C |
| ATOM | 4067 | CD | GLN | B | 8 | −0.773 | −20.443 | −1.582 | 1.00 | 85.99 | C |
| ATOM | 4068 | OE1 | GLN | B | 8 | 0.173 | −19.667 | −1.384 | 1.00 | 75.13 | O |
| ATOM | 4069 | NE2 | GLN | B | 8 | −1.778 | −20.597 | −0.722 | 1.00 | 72.32 | N |
| ATOM | 4072 | C | GLN | B | 8 | 1.088 | −19.372 | −5.626 | 1.00 | 58.36 | C |
| ATOM | 4073 | O | GLN | B | 8 | 2.173 | −19.925 | −5.383 | 1.00 | 63.24 | O |
| ATOM | 4074 | N | GLY | B | 9 | 0.939 | −18.422 | −6.553 | 1.00 | 48.93 | N |
| ATOM | 4076 | CA | GLY | B | 9 | 2.055 | −17.661 | −7.107 | 1.00 | 44.67 | C |
| ATOM | 4079 | C | GLY | B | 9 | 3.231 | −17.393 | −6.180 | 1.00 | 40.76 | C |
| ATOM | 4080 | O | GLY | B | 9 | 4.395 | −17.454 | −6.578 | 1.00 | 43.13 | O |
| ATOM | 4081 | N | SER | B | 10 | 2.932 | −17.051 | −4.939 | 1.00 | 37.29 | N |
| ATOM | 4083 | CA | SER | B | 10 | 3.960 | −16.610 | −4.023 | 1.00 | 41.69 | C |
| ATOM | 4085 | CB | SER | B | 10 | 3.377 | −16.250 | −2.649 | 1.00 | 39.95 | C |
| ATOM | 4088 | OG | SER | B | 10 | 2.739 | −17.386 | −2.098 | 1.00 | 57.71 | O |
| ATOM | 4090 | C | SER | B | 10 | 4.988 | −17.695 | −3.844 | 1.00 | 42.49 | C |
| ATOM | 4091 | O | SER | B | 10 | 6.130 | −17.383 | −3.540 | 1.00 | 40.24 | O |
| ATOM | 4092 | N | ILE | B | 11 | 4.559 | −18.957 | −3.917 | 1.00 | 40.05 | N |
| ATOM | 4094 | CA | ILE | B | 11 | 5.412 | −20.057 | −3.517 | 1.00 | 34.60 | C |
| ATOM | 4096 | CB | ILE | B | 11 | 4.585 | −21.352 | −3.379 | 1.00 | 39.02 | C |
| ATOM | 4098 | CG1 | ILE | B | 11 | 4.022 | −21.483 | −1.966 | 1.00 | 44.64 | C |
| ATOM | 4101 | CD1 | ILE | B | 11 | 2.995 | −22.594 | −1.871 | 1.00 | 54.68 | C |
| ATOM | 4105 | CG2 | ILE | B | 11 | 5.404 | −22.599 | −3.705 | 1.00 | 40.22 | C |
| ATOM | 4109 | C | ILE | B | 11 | 6.514 | −20.248 | −4.552 | 1.00 | 35.82 | C |
| ATOM | 4110 | O | ILE | B | 11 | 7.657 | −20.503 | −4.233 | 1.00 | 33.09 | O |
| ATOM | 4111 | N | LEU | B | 12 | 6.102 | −20.259 | −5.803 | 1.00 | 30.76 | N |
| ATOM | 4113 | CA | LEU | B | 12 | 7.015 | −20.194 | −6.910 | 1.00 | 34.20 | C |
| ATOM | 4115 | CB | LEU | B | 12 | 6.257 | −20.081 | −8.213 | 1.00 | 32.77 | C |
| ATOM | 4118 | CG | LEU | B | 12 | 6.901 | −21.035 | −9.219 | 1.00 | 58.60 | C |
| ATOM | 4120 | CD1 | LEU | B | 12 | 6.792 | −22.471 | −8.718 | 1.00 | 62.42 | C |
| ATOM | 4124 | CD2 | LEU | B | 12 | 6.287 | −20.915 | −10.598 | 1.00 | 56.22 | C |
| ATOM | 4128 | C | LEU | B | 12 | 8.043 | −19.093 | −6.814 | 1.00 | 38.18 | C |
| ATOM | 4129 | O | LEU | B | 12 | 9.176 | −19.309 | −7.245 | 1.00 | 40.54 | O |
| ATOM | 4130 | N | LYS | B | 13 | 7.645 | −17.914 | −6.333 | 1.00 | 33.12 | N |
| ATOM | 4132 | CA | LYS | B | 13 | 8.565 | −16.794 | −6.254 | 1.00 | 36.11 | C |
| ATOM | 4134 | CB | LYS | B | 13 | 7.841 | −15.491 | −5.937 | 1.00 | 34.78 | C |
| ATOM | 4137 | CG | LYS | B | 13 | 7.104 | −14.969 | −7.140 | 1.00 | 36.88 | C |
| ATOM | 4140 | CD | LYS | B | 13 | 5.775 | −14.399 | −6.684 | 1.00 | 57.19 | C |
| ATOM | 4143 | CE | LYS | B | 13 | 5.822 | −12.895 | −6.700 | 1.00 | 63.59 | C |
| ATOM | 4146 | NZ | LYS | B | 13 | 5.503 | −12.406 | −8.067 | 1.00 | 68.00 | N |
| ATOM | 4150 | C | LYS | B | 13 | 9.602 | −17.031 | −5.182 | 1.00 | 36.41 | C |
| ATOM | 4151 | O | LYS | B | 13 | 10.778 | −16.710 | −5.353 | 1.00 | 28.74 | O |
| ATOM | 4152 | N | LYS | B | 14 | 9.130 | −17.569 | −4.067 | 1.00 | 26.51 | N |
| ATOM | 4154 | CA | LYS | B | 14 | 10.004 | −17.868 | −2.967 | 1.00 | 29.89 | C |
| ATOM | 4156 | CB | LYS | B | 14 | 9.193 | −18.160 | −1.708 | 1.00 | 24.69 | C |
| ATOM | 4159 | CG | LYS | B | 14 | 8.360 | −16.976 | −1.220 | 1.00 | 26.53 | C |
| ATOM | 4162 | CD | LYS | B | 14 | 7.332 | −17.519 | −0.232 | 1.00 | 44.28 | C |
| ATOM | 4165 | CE | LYS | B | 14 | 6.508 | −16.436 | 0.437 | 1.00 | 49.44 | C |
| ATOM | 4168 | NZ | LYS | B | 14 | 5.552 | −17.097 | 1.380 | 1.00 | 62.80 | N |
| ATOM | 4172 | C | LYS | B | 14 | 10.979 | −19.001 | −3.274 | 1.00 | 33.19 | C |
| ATOM | 4173 | O | LYS | B | 14 | 12.063 | −18.986 | −2.714 | 1.00 | 41.15 | O |
| ATOM | 4174 | N | VAL | B | 15 | 10.606 | −19.953 | −4.131 | 1.00 | 26.43 | N |
| ATOM | 4176 | CA | VAL | B | 15 | 11.498 | −21.028 | −4.558 | 1.00 | 30.81 | C |
| ATOM | 4178 | CB | VAL | B | 15 | 10.732 | −22.116 | −5.357 | 1.00 | 39.80 | C |
| ATOM | 4180 | CG1 | VAL | B | 15 | 11.680 | −22.979 | −6.216 | 1.00 | 39.43 | C |
| ATOM | 4184 | CG2 | VAL | B | 15 | 9.972 | −23.022 | −4.415 | 1.00 | 39.45 | C |
| ATOM | 4188 | C | VAL | B | 15 | 12.639 | −20.493 | −5.423 | 1.00 | 21.33 | C |
| ATOM | 4189 | O | VAL | B | 15 | 13.780 | −20.897 | −5.262 | 1.00 | 33.37 | O |
| ATOM | 4190 | N | LEU | B | 16 | 12.326 | −19.592 | −6.342 | 1.00 | 25.40 | N |
| ATOM | 4192 | CA | LEU | B | 16 | 13.339 | −18.967 | −7.162 | 1.00 | 35.28 | C |
| ATOM | 4194 | CB | LEU | B | 16 | 12.727 | −18.344 | −8.411 | 1.00 | 29.68 | C |
| ATOM | 4197 | CG | LEU | B | 16 | 12.585 | −19.217 | −9.662 | 1.00 | 26.06 | C |
| ATOM | 4199 | CD1 | LEU | B | 16 | 11.657 | −20.391 | −9.539 | 1.00 | 34.18 | C |
| ATOM | 4203 | CD2 | LEU | B | 16 | 13.941 | −19.720 | −10.156 | 1.00 | 37.21 | C |
| ATOM | 4207 | C | LEU | B | 16 | 14.261 | −18.002 | −6.401 | 1.00 | 38.17 | C |
| ATOM | 4208 | O | LEU | B | 16 | 15.470 | −18.012 | −6.626 | 1.00 | 39.37 | O |
| ATOM | 4209 | N | GLU | B | 17 | 13.713 | −17.221 | −5.474 | 1.00 | 36.04 | N |
| ATOM | 4211 | CA | GLU | B | 17 | 14.529 | −16.436 | −4.535 | 1.00 | 38.36 | C |
| ATOM | 4213 | CB | GLU | B | 17 | 13.650 | −15.700 | −3.512 | 1.00 | 34.20 | C |
| ATOM | 4216 | CG | GLU | B | 17 | 13.254 | −14.283 | −3.936 | 1.00 | 59.27 | C |
| ATOM | 4219 | CD | GLU | B | 17 | 14.407 | −13.294 | −3.908 | 1.00 | 68.99 | C |
| ATOM | 4220 | OE1 | GLU | B | 17 | 14.562 | −12.532 | −4.884 | 1.00 | 75.19 | O |
| ATOM | 4221 | OE2 | GLU | B | 17 | 15.155 | −13.260 | −2.910 | 1.00 | 79.59 | O |
| ATOM | 4222 | C | GLU | B | 17 | 15.524 | −17.263 | −3.713 | 1.00 | 28.06 | C |
| ATOM | 4223 | O | GLU | B | 17 | 16.576 | −16.786 | −3.350 | 1.00 | 35.60 | O |
| ATOM | 4224 | N | ALA | B | 18 | 15.087 | −18.439 | −3.281 | 1.00 | 28.64 | N |
| ATOM | 4226 | CA | ALA | B | 18 | 15.908 | −19.359 | −2.540 | 1.00 | 30.82 | C |
| ATOM | 4228 | CB | ALA | B | 18 | 15.001 | −20.266 | −1.761 | 1.00 | 30.13 | C |
| ATOM | 4232 | C | ALA | B | 18 | 16.917 | −20.213 | −3.320 | 1.00 | 28.73 | C |
| ATOM | 4233 | O | ALA | B | 18 | 17.685 | −20.931 | −2.685 | 1.00 | 41.06 | O |
| ATOM | 4234 | N | LEU | B | 19 | 16.880 | −20.179 | −4.651 | 1.00 | 32.81 | N |
| ATOM | 4236 | CA | LEU | B | 19 | 17.800 | −20.941 | −5.475 | 1.00 | 32.46 | C |
| ATOM | 4238 | CB | LEU | B | 19 | 17.021 | −21.810 | −6.473 | 1.00 | 27.31 | C |
| ATOM | 4241 | CG | LEU | B | 19 | 16.259 | −23.047 | −6.005 | 1.00 | 34.96 | C |
| ATOM | 4243 | CD1 | LEU | B | 19 | 15.226 | −23.450 | −7.047 | 1.00 | 37.27 | C |
| ATOM | 4247 | CD2 | LEU | B | 19 | 17.263 | −24.121 | −5.800 | 1.00 | 42.04 | C |
| ATOM | 4251 | C | LEU | B | 19 | 18.792 | −20.090 | −6.289 | 1.00 | 39.50 | C |
| ATOM | 4252 | O | LEU | B | 19 | 19.849 | −20.596 | −6.674 | 1.00 | 34.97 | O |
| ATOM | 4253 | N | LYS | B | 20 | 18.386 | −18.886 | −6.698 | 1.00 | 30.94 | N |
| ATOM | 4255 | CA | LYS | B | 20 | 18.970 | −18.252 | −7.875 | 1.00 | 34.36 | C |
| ATOM | 4257 | CB | LYS | B | 20 | 18.083 | −17.103 | −8.382 | 1.00 | 26.32 | C |
| ATOM | 4260 | CG | LYS | B | 20 | 18.100 | −15.939 | −7.398 | 1.00 | 34.39 | C |
| ATOM | 4263 | CD | LYS | B | 20 | 17.250 | −14.768 | −7.851 | 1.00 | 45.49 | C |
| ATOM | 4266 | CE | LYS | B | 20 | 17.161 | −13.666 | −6.815 | 1.00 | 37.50 | C |
| ATOM | 4269 | NZ | LYS | B | 20 | 16.116 | −12.716 | −7.260 | 1.00 | 53.42 | N |
| ATOM | 4273 | C | LYS | B | 20 | 20.357 | −17.669 | −7.613 | 1.00 | 33.89 | C |
| ATOM | 4274 | O | LYS | B | 20 | 21.063 | −17.353 | −8.576 | 1.00 | 40.33 | O |
| ATOM | 4275 | N | ASP | B | 21 | 20.670 | −17.386 | −6.346 | 1.00 | 36.61 | N |
| ATOM | 4277 | CA | ASP | B | 21 | 22.012 | −16.930 | −5.966 | 1.00 | 45.87 | C |
| ATOM | 4279 | CB | ASP | B | 21 | 21.967 | −16.190 | −4.625 | 1.00 | 45.52 | C |
| ATOM | 4282 | CG | ASP | B | 21 | 21.333 | −14.799 | −4.715 | 1.00 | 48.93 | C |
| ATOM | 4283 | OD1 | ASP | B | 21 | 21.371 | −14.139 | −5.782 | 1.00 | 51.18 | O |
| ATOM | 4284 | OD2 | ASP | B | 21 | 20.747 | −14.299 | −3.725 | 1.00 | 55.93 | O |
| ATOM | 4285 | C | ASP | B | 21 | 23.033 | −18.098 | −5.840 | 1.00 | 46.04 | C |
| ATOM | 4286 | O | ASP | B | 21 | 24.233 | −17.880 | −5.782 | 1.00 | 41.37 | O |
| ATOM | 4287 | N | LEU | B | 22 | 22.533 | −19.325 | −5.773 | 1.00 | 41.72 | N |
| ATOM | 4289 | CA | LEU | B | 22 | 23.322 | −20.523 | −5.537 | 1.00 | 40.98 | C |
| ATOM | 4291 | CB | LEU | B | 22 | 22.518 | −21.461 | −4.650 | 1.00 | 36.81 | C |
| ATOM | 4294 | CG | LEU | B | 22 | 23.047 | −22.089 | −3.375 | 1.00 | 43.22 | C |
| ATOM | 4296 | CD1 | LEU | B | 22 | 24.196 | −21.364 | −2.757 | 1.00 | 37.61 | C |
| ATOM | 4300 | CD2 | LEU | B | 22 | 21.935 | −22.265 | −2.385 | 1.00 | 42.42 | C |
| ATOM | 4304 | C | LEU | B | 22 | 23.589 | −21.185 | −6.874 | 1.00 | 40.24 | C |
| ATOM | 4305 | O | LEU | B | 22 | 24.628 | −21.817 | −7.108 | 1.00 | 36.69 | O |
| ATOM | 4306 | N | ILE | B | 23 | 22.622 | −21.047 | −7.768 | 1.00 | 39.38 | N |
| ATOM | 4308 | CA | ILE | B | 23 | 22.716 | −21.723 | −9.045 | 1.00 | 41.18 | C |
| ATOM | 4310 | CB | ILE | B | 23 | 22.092 | −23.156 | −9.048 | 1.00 | 40.62 | C |
| ATOM | 4312 | CG1 | ILE | B | 23 | 21.334 | −23.395 | −10.350 | 1.00 | 48.21 | C |
| ATOM | 4315 | CD1 | ILE | B | 23 | 21.071 | −24.838 | −10.641 | 1.00 | 73.92 | C |
| ATOM | 4319 | CG2 | ILE | B | 23 | 21.212 | −23.435 | −7.872 | 1.00 | 44.59 | C |
| ATOM | 4323 | C | ILE | B | 23 | 22.172 | −20.887 | −10.188 | 1.00 | 38.64 | C |
| ATOM | 4324 | O | ILE | B | 23 | 21.080 | −20.346 | −10.127 | 1.00 | 45.72 | O |
| ATOM | 4325 | N | ASN | B | 24 | 22.889 | −20.925 | −11.297 | 1.00 | 39.05 | N |
| ATOM | 4327 | CA | ASN | B | 24 | 22.527 | −20.176 | −12.479 | 1.00 | 44.36 | C |
| ATOM | 4329 | CB | ASN | B | 24 | 23.808 | −19.734 | −13.179 | 1.00 | 44.96 | C |
| ATOM | 4332 | CG | ASN | B | 24 | 24.580 | −18.790 | −12.337 | 1.00 | 51.51 | C |
| ATOM | 4333 | OD1 | ASN | B | 24 | 25.614 | −19.146 | −11.775 | 1.00 | 66.78 | O |
| ATOM | 4334 | ND2 | ASN | B | 24 | 24.012 | −17.605 | −12.129 | 1.00 | 63.88 | N |
| ATOM | 4337 | C | ASN | B | 24 | 21.659 | −20.917 | −13.468 | 1.00 | 45.20 | C |
| ATOM | 4338 | O | ASN | B | 24 | 20.540 | −20.501 | −13.718 | 1.00 | 50.28 | O |
| ATOM | 4339 | N | GLU | B | 25 | 22.246 | −21.907 | −14.127 | 1.00 | 49.90 | N |
| ATOM | 4341 | CA | GLU | B | 25 | 21.547 | −22.775 | −15.064 | 1.00 | 51.07 | C |
| ATOM | 4343 | CB | GLU | B | 25 | 22.441 | −23.183 | −16.244 | 1.00 | 57.22 | C |
| ATOM | 4346 | CG | GLU | B | 25 | 23.582 | −22.258 | −16.607 | 1.00 | 59.04 | C |
| ATOM | 4349 | CD | GLU | B | 25 | 23.093 | −20.960 | −17.202 | 1.00 | 67.19 | C |
| ATOM | 4350 | OE1 | GLU | B | 25 | 22.332 | −21.006 | −18.197 | 1.00 | 55.72 | O |
| ATOM | 4351 | OE2 | GLU | B | 25 | 23.453 | −19.902 | −16.646 | 1.00 | 65.60 | O |
| ATOM | 4352 | C | GLU | B | 25 | 21.112 | −24.069 | −14.380 | 1.00 | 44.06 | C |
| ATOM | 4353 | O | GLU | B | 25 | 21.907 | −24.802 | −13.794 | 1.00 | 51.41 | O |
| ATOM | 4354 | N | ALA | B | 26 | 19.843 | −24.405 | −14.542 | 1.00 | 47.38 | N |
| ATOM | 4356 | CA | ALA | B | 26 | 19.293 | −25.565 | −13.856 | 1.00 | 40.76 | C |
| ATOM | 4358 | CB | ALA | B | 26 | 18.394 | −25.108 | −12.728 | 1.00 | 42.23 | C |
| ATOM | 4362 | C | ALA | B | 26 | 18.517 | −26.388 | −14.849 | 1.00 | 33.00 | C |
| ATOM | 4363 | O | ALA | B | 26 | 18.121 | −25.896 | −15.905 | 1.00 | 36.10 | O |
| ATOM | 4364 | N | CYS | B | 27 | 18.284 | −27.642 | −14.506 | 1.00 | 43.17 | N |
| ATOM | 4366 | CA | CYS | B | 27 | 17.482 | −28.518 | −15.353 | 1.00 | 36.62 | C |
| ATOM | 4368 | CB | CYS | B | 27 | 18.299 | −29.707 | −15.843 | 1.00 | 49.47 | C |
| ATOM | 4371 | SG | CYS | B | 27 | 17.640 | −30.565 | −17.293 | 1.00 | 53.79 | S |
| ATOM | 4372 | C | CYS | B | 27 | 16.275 | −28.944 | −14.518 | 1.00 | 36.93 | C |
| ATOM | 4373 | O | CYS | B | 27 | 16.380 | −29.545 | −13.442 | 1.00 | 37.11 | O |
| ATOM | 4374 | N | TRP | B | 28 | 15.102 | −28.531 | −14.973 | 1.00 | 34.62 | N |
| ATOM | 4376 | CA | TRP | B | 28 | 13.878 | −28.987 | −14.308 | 1.00 | 42.94 | C |
| ATOM | 4378 | CB | TRP | B | 28 | 12.759 | −27.943 | −14.446 | 1.00 | 41.61 | C |
| ATOM | 4381 | CG | TRP | B | 28 | 13.145 | −26.617 | −13.844 | 1.00 | 49.98 | C |
| ATOM | 4382 | CD1 | TRP | B | 28 | 14.149 | −25.818 | −14.265 | 1.00 | 60.57 | C |
| ATOM | 4384 | NE1 | TRP | B | 28 | 14.265 | −24.724 | −13.444 | 1.00 | 56.47 | N |
| ATOM | 4386 | CE2 | TRP | B | 28 | 13.349 | −24.825 | −12.435 | 1.00 | 31.46 | C |
| ATOM | 4387 | CD2 | TRP | B | 28 | 12.604 | −25.986 | −12.667 | 1.00 | 35.74 | C |
| ATOM | 4388 | CE3 | TRP | B | 28 | 11.592 | −26.312 | −11.760 | 1.00 | 56.87 | C |
| ATOM | 4390 | CZ3 | TRP | B | 28 | 11.298 | −25.422 | −10.721 | 1.00 | 48.06 | C |
| ATOM | 4392 | CH2 | TRP | B | 28 | 12.010 | −24.230 | −10.573 | 1.00 | 44.68 | C |
| ATOM | 4394 | CZ2 | TRP | B | 28 | 13.054 | −23.929 | −11.396 | 1.00 | 32.71 | C |
| ATOM | 4396 | C | TRP | B | 28 | 13.508 | −30.326 | −14.936 | 1.00 | 38.76 | C |
| ATOM | 4397 | O | TRP | B | 28 | 13.379 | −30.394 | −16.147 | 1.00 | 46.90 | O |
| ATOM | 4398 | N | ASP | B | 29 | 13.495 | −31.400 | −14.141 | 1.00 | 40.47 | N |
| ATOM | 4400 | CA | ASP | B | 29 | 12.847 | −32.642 | −14.533 | 1.00 | 35.08 | C |
| ATOM | 4402 | CB | ASP | B | 29 | 13.250 | −33.795 | −13.607 | 1.00 | 47.51 | C |
| ATOM | 4405 | CG | ASP | B | 29 | 14.427 | −34.558 | −14.085 | 1.00 | 63.16 | C |
| ATOM | 4406 | OD1 | ASP | B | 29 | 15.317 | −33.913 | −14.694 | 1.00 | 100.07 | O |
| ATOM | 4407 | OD2 | ASP | B | 29 | 14.554 | −35.781 | −13.826 | 1.00 | 77.58 | O |
| ATOM | 4408 | C | ASP | B | 29 | 11.340 | −32.519 | −14.321 | 1.00 | 27.02 | C |
| ATOM | 4409 | O | ASP | B | 29 | 10.862 | −32.561 | −13.166 | 1.00 | 26.11 | O |
| ATOM | 4410 | N | ILE | B | 30 | 10.589 | −32.582 | −15.410 | 1.00 | 28.74 | N |
| ATOM | 4412 | CA | ILE | B | 30 | 9.137 | −32.544 | −15.300 | 1.00 | 32.29 | C |
| ATOM | 4414 | CB | ILE | B | 30 | 8.545 | −31.467 | −16.240 | 1.00 | 33.11 | C |
| ATOM | 4416 | CG1 | ILE | B | 30 | 9.354 | −30.177 | −16.187 | 1.00 | 38.84 | C |
| ATOM | 4419 | CD1 | ILE | B | 30 | 8.908 | −29.258 | −15.089 | 1.00 | 49.83 | C |
| ATOM | 4423 | CG2 | ILE | B | 30 | 7.109 | −31.179 | −15.872 | 1.00 | 44.37 | C |
| ATOM | 4427 | C | ILE | B | 30 | 8.635 | −33.926 | −15.678 | 1.00 | 31.18 | C |
| ATOM | 4428 | O | ILE | B | 30 | 8.781 | −34.365 | −16.795 | 1.00 | 31.21 | O |
| ATOM | 4429 | N | SER | B | 31 | 8.018 | −34.595 | −14.727 | 1.00 | 24.52 | N |
| ATOM | 4431 | CA | SER | B | 31 | 7.352 | −35.861 | −14.968 | 1.00 | 27.93 | C |
| ATOM | 4433 | CB | SER | B | 31 | 8.059 | −36.994 | −14.187 | 1.00 | 16.56 | C |
| ATOM | 4436 | OG | SER | B | 31 | 7.875 | −36.890 | −12.790 | 1.00 | 28.40 | O |
| ATOM | 4438 | C | SER | B | 31 | 5.900 | −35.751 | −14.483 | 1.00 | 28.16 | C |
| ATOM | 4439 | O | SER | B | 31 | 5.476 | −34.714 | −13.981 | 1.00 | 30.79 | O |
| ATOM | 4440 | N | SER | B | 32 | 5.211 | −36.890 | −14.489 | 1.00 | 26.94 | N |
| ATOM | 4442 | CA | SER | B | 32 | 3.778 | −36.937 | −14.249 | 1.00 | 28.25 | C |
| ATOM | 4444 | CB | SER | B | 32 | 3.143 | −38.109 | −15.022 | 1.00 | 27.40 | C |
| ATOM | 4447 | OG | SER | B | 32 | 3.551 | −39.372 | −14.511 | 1.00 | 36.37 | O |
| ATOM | 4449 | C | SER | B | 32 | 3.596 | −37.023 | −12.736 | 1.00 | 27.83 | C |
| ATOM | 4450 | O | SER | B | 32 | 2.563 | −36.635 | −12.199 | 1.00 | 38.62 | O |
| ATOM | 4451 | N | SER | B | 33 | 4.671 | −37.369 | −12.036 | 1.00 | 30.02 | N |
| ATOM | 4453 | CA | SER | B | 33 | 4.666 | −37.345 | −10.580 | 1.00 | 30.38 | C |
| ATOM | 4455 | CB | SER | B | 33 | 5.505 | −38.501 | −10.010 | 1.00 | 35.76 | C |
| ATOM | 4458 | OG | SER | B | 33 | 6.867 | −38.155 | −9.918 | 1.00 | 45.44 | O |
| ATOM | 4460 | C | SER | B | 33 | 5.039 | −35.969 | −10.002 | 1.00 | 28.89 | C |
| ATOM | 4461 | O | SER | B | 33 | 4.833 | −35.680 | −8.852 | 1.00 | 36.45 | O |
| ATOM | 4462 | N | GLY | B | 34 | 5.491 | −35.066 | −10.843 | 1.00 | 34.64 | N |
| ATOM | 4464 | CA | GLY | B | 34 | 5.761 | −33.731 | −10.399 | 1.00 | 31.96 | C |
| ATOM | 4467 | C | GLY | B | 34 | 7.071 | −33.183 | −10.985 | 1.00 | 35.12 | C |
| ATOM | 4468 | O | GLY | B | 34 | 7.657 | −33.709 | −11.915 | 1.00 | 37.51 | O |
| ATOM | 4469 | N | VAL | B | 35 | 7.523 | −32.095 | −10.378 | 1.00 | 36.53 | N |
| ATOM | 4471 | CA | VAL | B | 35 | 8.724 | −31.351 | −10.745 | 1.00 | 39.06 | C |
| ATOM | 4473 | CB | VAL | B | 35 | 8.376 | −29.852 | −10.765 | 1.00 | 41.40 | C |
| ATOM | 4475 | CG1 | VAL | B | 35 | 9.600 | −28.971 | −10.902 | 1.00 | 36.66 | C |
| ATOM | 4479 | CG2 | VAL | B | 35 | 7.394 | −29.540 | −11.877 | 1.00 | 36.74 | C |
| ATOM | 4483 | C | VAL | B | 35 | 9.877 | −31.585 | −9.751 | 1.00 | 36.11 | C |
| ATOM | 4484 | O | VAL | B | 35 | 9.661 | −31.661 | −8.561 | 1.00 | 33.94 | O |
| ATOM | 4485 | N | ASN | B | 36 | 11.098 | −31.751 | −10.262 | 1.00 | 35.94 | N |
| ATOM | 4487 | CA | ASN | B | 36 | 12.270 | −32.121 | −9.454 | 1.00 | 31.52 | C |
| ATOM | 4489 | CB | ASN | B | 36 | 12.631 | −33.606 | −9.604 | 1.00 | 25.29 | C |
| ATOM | 4492 | CG | ASN | B | 36 | 11.997 | −34.488 | −8.558 | 1.00 | 42.77 | C |
| ATOM | 4493 | OD1 | ASN | B | 36 | 12.473 | −34.557 | −7.433 | 1.00 | 62.23 | O |
| ATOM | 4494 | ND2 | ASN | B | 36 | 10.937 | −35.204 | −8.934 | 1.00 | 63.14 | N |
| ATOM | 4497 | C | ASN | B | 36 | 13.490 | −31.340 | −9.972 | 1.00 | 36.42 | C |
| ATOM | 4498 | O | ASN | B | 36 | 13.761 | −31.287 | −11.150 | 1.00 | 27.92 | O |
| ATOM | 4499 | N | LEU | B | 37 | 14.256 | −30.759 | −9.075 | 1.00 | 33.88 | N |
| ATOM | 4501 | CA | LEU | B | 37 | 15.417 | −30.040 | −9.500 | 1.00 | 32.93 | C |
| ATOM | 4503 | CB | LEU | B | 37 | 15.187 | −28.532 | −9.477 | 1.00 | 31.98 | C |
| ATOM | 4506 | CG | LEU | B | 37 | 16.365 | −27.722 | −10.056 | 1.00 | 46.56 | C |
| ATOM | 4508 | CD1 | LEU | B | 37 | 15.900 | −26.380 | −10.583 | 1.00 | 54.87 | C |
| ATOM | 4512 | CD2 | LEU | B | 37 | 17.424 | −27.489 | −8.994 | 1.00 | 51.53 | C |
| ATOM | 4516 | C | LEU | B | 37 | 16.497 | −30.495 | −8.534 | 1.00 | 39.17 | C |
| ATOM | 4517 | O | LEU | B | 37 | 16.302 | −30.649 | −7.317 | 1.00 | 37.86 | O |
| ATOM | 4518 | N | GLN | B | 38 | 17.593 | −30.884 | −9.143 | 1.00 | 30.61 | N |
| ATOM | 4520 | CA | GLN | B | 38 | 18.749 | −31.311 | −8.404 | 1.00 | 30.92 | C |
| ATOM | 4522 | CB | GLN | B | 38 | 18.775 | −32.840 | −8.349 | 1.00 | 31.46 | C |
| ATOM | 4525 | CG | GLN | B | 38 | 19.556 | −33.368 | −7.168 | 1.00 | 47.90 | C |
| ATOM | 4528 | CD | GLN | B | 38 | 19.533 | −34.882 | −7.105 | 1.00 | 59.33 | C |
| ATOM | 4529 | OE1 | GLN | B | 38 | 20.525 | −35.516 | −7.429 | 1.00 | 68.58 | O |
| ATOM | 4530 | NE2 | GLN | B | 38 | 18.405 | −35.458 | −6.704 | 1.00 | 59.43 | N |
| ATOM | 4533 | C | GLN | B | 38 | 20.009 | −30.789 | −9.083 | 1.00 | 40.00 | C |
| ATOM | 4534 | O | GLN | B | 38 | 20.222 | −31.033 | −10.294 | 1.00 | 37.58 | O |
| ATOM | 4535 | N | SER | B | 39 | 20.845 | −30.101 | −8.298 | 1.00 | 40.08 | N |
| ATOM | 4537 | CA | SER | B | 39 | 22.105 | −29.540 | −8.796 | 1.00 | 40.76 | C |
| ATOM | 4539 | CB | SER | B | 39 | 21.864 | −28.278 | −9.633 | 1.00 | 44.00 | C |
| ATOM | 4542 | OG | SER | B | 39 | 23.103 | −27.752 | −10.083 | 1.00 | 46.78 | O |
| ATOM | 4544 | C | SER | B | 39 | 23.072 | −29.173 | −7.679 | 1.00 | 43.99 | C |
| ATOM | 4545 | O | SER | B | 39 | 22.662 | −28.764 | −6.576 | 1.00 | 30.61 | O |
| ATOM | 4546 | N | MET | B | 40 | 24.362 | −29.221 | −8.011 | 1.00 | 38.35 | N |
| ATOM | 4548 | CA | MET | B | 40 | 25.361 | −28.531 | −7.200 | 1.00 | 33.37 | C |
| ATOM | 4550 | CB | MET | B | 40 | 26.782 | −28.858 | −7.649 | 1.00 | 35.94 | C |
| ATOM | 4553 | CG | MET | B | 40 | 27.158 | −30.305 | −7.531 | 1.00 | 47.23 | C |
| ATOM | 4556 | SD | MET | B | 40 | 27.373 | −30.806 | −5.839 | 1.00 | 46.87 | S |
| ATOM | 4557 | CE | MET | B | 40 | 26.883 | −32.517 | −6.040 | 1.00 | 59.47 | C |
| ATOM | 4561 | C | MET | B | 40 | 25.157 | −27.057 | −7.463 | 1.00 | 22.36 | C |
| ATOM | 4562 | O | MET | B | 40 | 24.686 | −26.658 | −8.525 | 1.00 | 28.88 | O |
| ATOM | 4563 | N | ASP | B | 41 | 25.626 | −26.253 | −6.525 | 1.00 | 19.18 | N |
| ATOM | 4565 | CA | ASP | B | 41 | 25.734 | −24.815 | −6.750 | 1.00 | 28.10 | C |
| ATOM | 4567 | CB | ASP | B | 41 | 25.811 | −24.146 | −5.382 | 1.00 | 30.28 | C |
| ATOM | 4570 | CG | ASP | B | 41 | 27.144 | −24.386 | −4.709 | 1.00 | 36.90 | C |
| ATOM | 4571 | OD1 | ASP | B | 41 | 28.128 | −24.699 | −5.413 | 1.00 | 54.67 | O |
| ATOM | 4572 | OD2 | ASP | B | 41 | 27.296 | −24.304 | −3.490 | 1.00 | 29.39 | O |
| ATOM | 4573 | C | ASP | B | 41 | 26.978 | −24.457 | −7.609 | 1.00 | 30.50 | C |
| ATOM | 4574 | O | ASP | B | 41 | 27.779 | −25.340 | −7.916 | 1.00 | 23.50 | O |
| ATOM | 4575 | N | SER | B | 42 | 27.089 | −23.192 | −8.038 | 1.00 | 37.85 | N |
| ATOM | 4577 | CA | SER | B | 42 | 28.033 | −22.773 | −9.086 | 1.00 | 42.18 | C |
| ATOM | 4579 | CB | SER | B | 42 | 27.654 | −21.377 | −9.579 | 1.00 | 49.53 | C |
| ATOM | 4582 | OG | SER | B | 42 | 27.590 | −20.453 | −8.504 | 1.00 | 54.67 | O |
| ATOM | 4584 | C | SER | B | 42 | 29.516 | −22.779 | −8.664 | 1.00 | 37.57 | C |
| ATOM | 4585 | O | SER | B | 42 | 30.413 | −22.689 | −9.506 | 1.00 | 45.26 | O |
| ATOM | 4586 | N | SER | B | 43 | 29.756 | −22.973 | −7.372 | 1.00 | 38.08 | N |
| ATOM | 4588 | CA | SER | B | 43 | 31.102 | −23.148 | −6.815 | 1.00 | 39.05 | C |
| ATOM | 4590 | CB | SER | B | 43 | 31.234 | −22.352 | −5.517 | 1.00 | 41.48 | C |
| ATOM | 4593 | OG | SER | B | 43 | 30.869 | −20.999 | −5.744 | 1.00 | 55.87 | O |
| ATOM | 4595 | C | SER | B | 43 | 31.434 | −24.611 | −6.547 | 1.00 | 30.84 | C |
| ATOM | 4596 | O | SER | B | 43 | 32.440 | −24.925 | −5.908 | 1.00 | 36.49 | O |
| ATOM | 4597 | N | HIS | B | 44 | 30.575 | −25.519 | −7.005 | 1.00 | 32.79 | N |
| ATOM | 4599 | CA | HIS | B | 44 | 30.748 | −26.930 | −6.667 | 1.00 | 32.12 | C |
| ATOM | 4601 | CB | HIS | B | 44 | 31.992 | −27.508 | −7.367 | 1.00 | 35.27 | C |
| ATOM | 4604 | CG | HIS | B | 44 | 31.790 | −28.900 | −7.875 | 1.00 | 30.77 | C |
| ATOM | 4605 | ND1 | HIS | B | 44 | 31.167 | −29.143 | −9.078 | 1.00 | 30.70 | N |
| ATOM | 4607 | CE1 | HIS | B | 44 | 31.061 | −30.443 | −9.258 | 1.00 | 27.36 | C |
| ATOM | 4609 | NE2 | HIS | B | 44 | 31.516 | −31.052 | −8.181 | 1.00 | 28.20 | N |
| ATOM | 4611 | CD2 | HIS | B | 44 | 31.987 | −30.110 | −7.297 | 1.00 | 32.00 | C |
| ATOM | 4613 | C | HIS | B | 44 | 30.888 | −27.251 | −5.183 | 1.00 | 26.81 | C |
| ATOM | 4614 | O | HIS | B | 44 | 31.481 | −28.260 | −4.831 | 1.00 | 31.07 | O |
| ATOM | 4615 | N | VAL | B | 45 | 30.285 | −26.471 | −4.298 | 1.00 | 31.71 | N |
| ATOM | 4617 | CA | VAL | B | 45 | 30.395 | −26.775 | −2.863 | 1.00 | 35.97 | C |
| ATOM | 4619 | CB | VAL | B | 45 | 30.511 | −25.472 | −2.008 | 1.00 | 35.78 | C |
| ATOM | 4621 | CG1 | VAL | B | 45 | 30.613 | −25.795 | −0.498 | 1.00 | 36.16 | C |
| ATOM | 4625 | CG2 | VAL | B | 45 | 31.626 | −24.555 | −2.503 | 1.00 | 39.03 | C |
| ATOM | 4629 | C | VAL | B | 45 | 29.177 | −27.550 | −2.354 | 1.00 | 34.11 | C |
| ATOM | 4630 | O | VAL | B | 45 | 29.293 | −28.683 | −1.887 | 1.00 | 31.76 | O |
| ATOM | 4631 | N | SER | B | 46 | 28.013 | −26.890 | −2.378 | 1.00 | 35.48 | N |
| ATOM | 4633 | CA | SER | B | 46 | 26.791 | −27.495 | −1.829 | 1.00 | 32.53 | C |
| ATOM | 4635 | CB | SER | B | 46 | 26.017 | −26.453 | −1.024 | 1.00 | 32.94 | C |
| ATOM | 4638 | OG | SER | B | 46 | 25.306 | −25.558 | −1.865 | 1.00 | 25.85 | O |
| ATOM | 4640 | C | SER | B | 46 | 25.865 | −28.128 | −2.875 | 1.00 | 28.07 | C |
| ATOM | 4641 | O | SER | B | 46 | 25.948 | −27.889 | −4.081 | 1.00 | 29.16 | O |
| ATOM | 4642 | N | LEU | B | 47 | 24.943 | −28.944 | −2.386 | 1.00 | 36.09 | N |
| ATOM | 4644 | CA | LEU | B | 47 | 23.982 | −29.586 | −3.271 | 1.00 | 33.79 | C |
| ATOM | 4646 | CB | LEU | B | 47 | 24.138 | −31.098 | −3.169 | 1.00 | 33.14 | C |
| ATOM | 4649 | CG | LEU | B | 47 | 23.002 | −31.968 | −3.724 | 1.00 | 38.88 | C |
| ATOM | 4651 | CD1 | LEU | B | 47 | 22.977 | −31.908 | −5.221 | 1.00 | 34.91 | C |
| ATOM | 4655 | CD2 | LEU | B | 47 | 23.240 | −33.390 | −3.333 | 1.00 | 57.49 | C |
| ATOM | 4659 | C | LEU | B | 47 | 22.544 | −29.161 | −2.898 | 1.00 | 36.95 | C |
| ATOM | 4660 | O | LEU | B | 47 | 22.199 | −29.144 | −1.724 | 1.00 | 28.39 | O |
| ATOM | 4661 | N | VAL | B | 48 | 21.712 | −28.877 | −3.897 | 1.00 | 35.82 | N |
| ATOM | 4663 | CA | VAL | B | 48 | 20.306 | −28.514 | −3.699 | 1.00 | 34.62 | C |
| ATOM | 4665 | CB | VAL | B | 48 | 19.912 | −27.194 | −4.464 | 1.00 | 38.43 | C |
| ATOM | 4667 | CG1 | VAL | B | 48 | 18.595 | −26.662 | −3.955 | 1.00 | 40.29 | C |
| ATOM | 4671 | CG2 | VAL | B | 48 | 20.934 | −26.110 | −4.347 | 1.00 | 37.23 | C |
| ATOM | 4675 | C | VAL | B | 48 | 19.408 | −29.559 | −4.362 | 1.00 | 37.69 | C |
| ATOM | 4676 | O | VAL | B | 48 | 19.719 | −30.025 | −5.460 | 1.00 | 33.57 | O |
| ATOM | 4677 | N | GLN | B | 49 | 18.258 | −29.825 | −3.744 | 1.00 | 29.25 | N |
| ATOM | 4679 | CA | GLN | B | 49 | 17.261 | −30.728 | −4.271 | 1.00 | 25.57 | C |
| ATOM | 4681 | CB | GLN | B | 49 | 17.452 | −32.101 | −3.636 | 1.00 | 34.19 | C |
| ATOM | 4684 | CG | GLN | B | 49 | 16.899 | −33.225 | −4.456 | 1.00 | 47.79 | C |
| ATOM | 4687 | CD | GLN | B | 49 | 15.501 | −33.518 | −4.022 | 1.00 | 55.36 | C |
| ATOM | 4688 | OE1 | GLN | B | 49 | 15.280 | −33.757 | −2.836 | 1.00 | 56.02 | O |
| ATOM | 4689 | NE2 | GLN | B | 49 | 14.537 | −33.419 | −4.952 | 1.00 | 53.01 | N |
| ATOM | 4692 | C | GLN | B | 49 | 15.857 | −30.214 | −3.953 | 1.00 | 29.37 | C |
| ATOM | 4693 | O | GLN | B | 49 | 15.474 | −30.155 | −2.779 | 1.00 | 28.44 | O |
| ATOM | 4694 | N | LEU | B | 50 | 15.140 | −29.805 | −5.007 | 1.00 | 27.87 | N |
| ATOM | 4696 | CA | LEU | B | 50 | 13.764 | −29.359 | −4.925 | 1.00 | 25.04 | C |
| ATOM | 4698 | CB | LEU | B | 50 | 13.584 | −20.039 | −5.668 | 1.00 | 33.84 | C |
| ATOM | 4701 | CG | LEU | B | 50 | 12.110 | −27.617 | −5.890 | 1.00 | 25.25 | C |
| ATOM | 4703 | CD1 | LEU | B | 50 | 11.553 | −26.952 | −4.656 | 1.00 | 30.67 | C |
| ATOM | 4707 | CD2 | LEU | B | 50 | 11.932 | −26.724 | −7.064 | 1.00 | 30.10 | C |
| ATOM | 4711 | C | LEU | B | 50 | 12.806 | −30.438 | −5.494 | 1.00 | 32.14 | C |
| ATOM | 4712 | O | LEU | B | 50 | 13.046 | −31.051 | −6.538 | 1.00 | 26.91 | O |
| ATOM | 4713 | N | THR | B | 51 | 11.704 | −30.650 | −4.787 | 1.00 | 27.17 | N |
| ATOM | 4715 | CA | THR | B | 51 | 10.636 | −31.506 | −5.257 | 1.00 | 30.25 | C |
| ATOM | 4717 | CB | THR | B | 51 | 10.653 | −32.786 | −4.408 | 1.00 | 38.00 | C |
| ATOM | 4719 | OG1 | THR | B | 51 | 11.936 | −33.408 | −4.529 | 1.00 | 41.33 | O |
| ATOM | 4721 | CG2 | THR | B | 51 | 9.682 | −33.837 | −4.934 | 1.00 | 39.06 | C |
| ATOM | 4725 | C | THR | B | 51 | 9.288 | −30.815 | −5.111 | 1.00 | 32.43 | C |
| ATOM | 4726 | O | THR | B | 51 | 8.959 | −30.330 | −4.007 | 1.00 | 31.41 | O |
| ATOM | 4727 | N | LEU | B | 52 | 8.536 | −30.720 | −6.218 | 1.00 | 30.31 | N |
| ATOM | 4729 | CA | LEU | B | 52 | 7.148 | −30.204 | −6.179 | 1.00 | 32.55 | C |
| ATOM | 4731 | CB | LEU | B | 52 | 7.014 | −28.834 | −6.857 | 1.00 | 29.24 | C |
| ATOM | 4734 | CG | LEU | B | 52 | 7.883 | −27.647 | −6.362 | 1.00 | 27.02 | C |
| ATOM | 4736 | CD1 | LEU | B | 52 | 7.669 | −26.454 | −7.266 | 1.00 | 38.32 | C |
| ATOM | 4740 | CD2 | LEU | B | 52 | 7.605 | −27.272 | −4.920 | 1.00 | 37.20 | C |
| ATOM | 4744 | C | LEU | B | 52 | 6.243 | −31.267 | −6.820 | 1.00 | 27.42 | C |
| ATOM | 4745 | O | LEU | B | 52 | 6.275 | −31.530 | −8.014 | 1.00 | 36.22 | O |
| ATOM | 4746 | N | ARG | B | 53 | 5.546 | −31.995 | −5.980 | 1.00 | 24.22 | N |
| ATOM | 4748 | CA | ARG | B | 53 | 4.617 | −33.035 | −6.454 | 1.00 | 32.61 | C |
| ATOM | 4750 | CB | ARG | B | 53 | 4.036 | −33.775 | −5.253 | 1.00 | 34.03 | C |
| ATOM | 4753 | CG | ARG | B | 53 | 5.082 | −34.607 | −4.523 | 1.00 | 39.43 | C |
| ATOM | 4756 | CD | ARG | B | 53 | 4.525 | −35.301 | −3.291 | 1.00 | 43.63 | C |
| ATOM | 4759 | NE | ARG | B | 53 | 4.896 | −34.543 | −2.104 | 1.00 | 55.61 | N |
| ATOM | 4761 | CZ | ARG | B | 53 | 4.029 | −33.974 | −1.283 | 1.00 | 68.95 | C |
| ATOM | 4762 | NH1 | ARG | B | 53 | 2.722 | −34.097 | −1.520 | 1.00 | 68.07 | N |
| ATOM | 4765 | NH2 | ARG | B | 53 | 4.478 | −33.308 | −0.215 | 1.00 | 44.71 | N |
| ATOM | 4768 | C | ARG | B | 53 | 3.443 | −32.471 | −7.245 | 1.00 | 26.45 | C |
| ATOM | 4769 | O | ARG | B | 53 | 2.995 | −31.337 | −7.016 | 1.00 | 28.74 | O |
| ATOM | 4770 | N | SER | B | 54 | 3.010 | −33.268 | −8.213 | 1.00 | 25.55 | N |
| ATOM | 4772 | CA | SER | B | 54 | 1.957 | −32.909 | −9.158 | 1.00 | 28.71 | C |
| ATOM | 4774 | CB | SER | B | 54 | 1.679 | −34.085 | −10.121 | 1.00 | 29.63 | C |
| ATOM | 4777 | OG | SER | B | 54 | 1.261 | −35.209 | −9.354 | 1.00 | 31.60 | O |
| ATOM | 4779 | C | SER | B | 54 | 0.682 | −32.583 | −8.365 | 1.00 | 29.00 | C |
| ATOM | 4780 | O | SER | B | 54 | −0.004 | −31.614 | −8.698 | 1.00 | 28.81 | O |
| ATOM | 4781 | N | GLU | B | 55 | 0.458 | −33.279 | −7.247 | 1.00 | 27.06 | N |
| ATOM | 4783 | CA | GLU | B | 55 | −0.780 | −33.086 | −6.483 | 1.00 | 39.56 | C |
| ATOM | 4785 | CB | GLU | B | 55 | −0.974 | −34.179 | −5.407 | 1.00 | 40.29 | C |
| ATOM | 4788 | CG | GLU | B | 55 | −0.647 | −35.586 | −5.879 | 1.00 | 52.51 | C |
| ATOM | 4791 | CD | GLU | B | 55 | 0.738 | −36.002 | −5.424 | 1.00 | 59.40 | C |
| ATOM | 4792 | OE1 | GLU | B | 55 | 0.904 | −36.325 | −4.227 | 1.00 | 80.06 | O |
| ATOM | 4793 | OE2 | GLU | B | 55 | 1.668 | −35.949 | −6.244 | 1.00 | 47.81 | O |
| ATOM | 4794 | C | GLU | B | 55 | −0.831 | −31.714 | −5.816 | 1.00 | 38.91 | C |
| ATOM | 4795 | O | GLU | B | 55 | −1.849 | −31.355 | −5.232 | 1.00 | 28.39 | O |
| ATOM | 4796 | N | GLY | B | 56 | 0.296 | −31.008 | −5.790 | 1.00 | 32.39 | N |
| ATOM | 4798 | CA | GLY | B | 56 | 0.380 | −29.792 | −4.999 | 1.00 | 29.68 | C |
| ATOM | 4801 | C | GLY | B | 56 | −0.112 | −28.632 | −5.835 | 1.00 | 35.81 | C |
| ATOM | 4802 | O | GLY | B | 56 | −0.367 | −27.541 | −5.321 | 1.00 | 40.30 | O |
| ATOM | 4803 | N | PHE | B | 57 | −0.229 | −28.890 | −7.137 | 1.00 | 33.19 | N |
| ATOM | 4805 | CA | PHE | B | 57 | −0.762 | −27.941 | −8.097 | 1.00 | 30.12 | C |
| ATOM | 4807 | CB | PHE | B | 57 | −0.097 | −28.099 | −9.480 | 1.00 | 33.83 | C |
| ATOM | 4810 | CG | PHE | B | 57 | 1.369 | −27.792 | −9.503 | 1.00 | 28.72 | C |
| ATOM | 4811 | CD1 | PHE | B | 57 | 2.293 | −28.715 | −9.008 | 1.00 | 35.07 | C |
| ATOM | 4813 | CE1 | PHE | B | 57 | 3.661 | −28.445 | −9.043 | 1.00 | 36.47 | C |
| ATOM | 4815 | CZ | PHE | B | 57 | 4.106 | −27.220 | −9.525 | 1.00 | 23.57 | C |
| ATOM | 4817 | CE2 | PHE | B | 57 | 3.201 | −26.315 | −10.047 | 1.00 | 37.86 | C |
| ATOM | 4819 | CD2 | PHE | B | 57 | 1.829 | −26.599 | −10.028 | 1.00 | 34.00 | C |
| ATOM | 4821 | C | PHE | B | 57 | −2.240 | −28.262 | −8.273 | 1.00 | 30.06 | C |
| ATOM | 4822 | O | PHE | B | 57 | −2.676 | −29.411 | −8.166 | 1.00 | 34.39 | O |
| ATOM | 4823 | N | ASP | B | 58 | −3.010 | −27.226 | −8.547 | 1.00 | 37.19 | N |
| ATOM | 4825 | CA | ASP | B | 58 | −4.438 | −27.405 | −8.604 | 1.00 | 43.83 | C |
| ATOM | 4827 | CB | ASP | B | 58 | −5.165 | −26.248 | −7.921 | 1.00 | 52.40 | C |
| ATOM | 4830 | CG | ASP | B | 58 | −5.050 | −24.969 | −8.657 | 1.00 | 50.38 | C |
| ATOM | 4831 | OD1 | ASP | B | 58 | −5.075 | −25.014 | −9.897 | 1.00 | 84.13 | O |
| ATOM | 4832 | OD2 | ASP | B | 58 | −4.998 | −23.862 | −8.093 | 1.00 | 49.51 | O |
| ATOM | 4833 | C | ASP | B | 58 | −4.951 | −27.867 | −9.975 | 1.00 | 42.82 | C |
| ATOM | 4834 | O | ASP | B | 58 | −6.016 | −28.445 | −10.057 | 1.00 | 56.83 | O |
| ATOM | 4835 | N | THR | B | 59 | −4.095 | −27.798 | −10.996 | 1.00 | 46.57 | N |
| ATOM | 4837 | CA | THR | B | 59 | −4.179 | −28.642 | −12.198 | 1.00 | 41.14 | C |
| ATOM | 4839 | CB | THR | B | 59 | −4.724 | −27.829 | −13.415 | 1.00 | 43.28 | C |
| ATOM | 4841 | OG1 | THR | B | 59 | −6.025 | −27.291 | −13.126 | 1.00 | 58.98 | O |
| ATOM | 4843 | CG2 | THR | B | 59 | −4.981 | −28.740 | −14.592 | 1.00 | 56.48 | C |
| ATOM | 4847 | C | THR | B | 59 | −2.784 | −29.144 | −12.541 | 1.00 | 36.06 | C |
| ATOM | 4848 | O | THR | B | 59 | −1.795 | −28.435 | −12.334 | 1.00 | 38.78 | O |
| ATOM | 4849 | N | TYR | B | 60 | −2.699 | −30.327 | −13.133 | 1.00 | 29.52 | N |
| ATOM | 4851 | CA | TYR | B | 60 | −1.401 | −30.905 | −13.518 | 1.00 | 34.25 | C |
| ATOM | 4853 | CB | TYR | B | 60 | −0.589 | −31.405 | −12.315 | 1.00 | 31.77 | C |
| ATOM | 4856 | CG | TYR | B | 60 | 0.892 | −31.642 | −12.625 | 1.00 | 29.54 | C |
| ATOM | 4857 | CD1 | TYR | B | 60 | 1.847 | −30.763 | −12.143 | 1.00 | 39.25 | C |
| ATOM | 4859 | CE1 | TYR | B | 60 | 3.191 | −30.960 | −12.377 | 1.00 | 22.43 | C |
| ATOM | 4861 | CZ | TYR | B | 60 | 3.630 | −32.061 | −13.095 | 1.00 | 34.60 | C |
| ATOM | 4862 | OH | TYR | B | 60 | 4.982 | −32.191 | −13.399 | 1.00 | 28.72 | O |
| ATOM | 4864 | CE2 | TYR | B | 60 | 2.706 | −32.972 | −13.574 | 1.00 | 32.71 | C |
| ATOM | 4866 | CD2 | TYR | B | 60 | 1.345 | −32.770 | −13.349 | 1.00 | 23.69 | C |
| ATOM | 4868 | C | TYR | B | 60 | −1.465 | −32.028 | −14.533 | 1.00 | 33.39 | C |
| ATOM | 4869 | O | TYR | B | 60 | −2.126 | −33.041 | −14.343 | 1.00 | 38.57 | O |
| ATOM | 4870 | N | ARG | B | 61 | −0.711 | −31.817 | −15.600 | 1.00 | 33.59 | N |
| ATOM | 4872 | CA | ARG | B | 61 | −0.756 | −32.656 | −16.776 | 1.00 | 39.74 | C |
| ATOM | 4874 | CB | ARG | B | 61 | −1.604 | −32.005 | −17.873 | 1.00 | 38.48 | C |
| ATOM | 4877 | CG | ARG | B | 61 | −1.732 | −32.911 | −19.106 | 1.00 | 58.00 | C |
| ATOM | 4880 | CD | ARG | B | 61 | −2.215 | −32.239 | −20.394 | 1.00 | 60.88 | C |
| ATOM | 4883 | NE | ARG | B | 61 | −2.040 | −33.088 | −21.576 | 1.00 | 57.95 | N |
| ATOM | 4885 | CZ | ARG | B | 61 | −2.433 | −32.762 | −22.811 | 1.00 | 66.81 | C |
| ATOM | 4886 | NH1 | ARG | B | 61 | −2.999 | −31.587 | −23.070 | 1.00 | 61.59 | N |
| ATOM | 4889 | NH2 | ARG | B | 61 | −2.218 | −33.596 | −23.815 | 1.00 | 58.17 | N |
| ATOM | 4892 | C | ARG | B | 61 | 0.659 | −32.743 | −17.290 | 1.00 | 32.89 | C |
| ATOM | 4893 | O | ARG | B | 61 | 1.239 | −31.706 | −17.603 | 1.00 | 35.53 | O |
| ATOM | 4894 | N | CYS | B | 62 | 1.172 | −33.954 | −17.481 | 1.00 | 39.97 | N |
| ATOM | 4896 | CA | CYS | B | 62 | 2.468 | −34.158 | −18.121 | 1.00 | 33.07 | C |
| ATOM | 4898 | CB | CYS | B | 62 | 3.581 | −34.209 | −17.071 | 1.00 | 42.11 | C |
| ATOM | 4901 | SG | CYS | B | 62 | 5.255 | −34.236 | −17.841 | 1.00 | 40.93 | S |
| ATOM | 4902 | C | CYS | B | 62 | 2.529 | −35.444 | −18.952 | 1.00 | 36.19 | C |
| ATOM | 4903 | O | CYS | B | 62 | 2.551 | −36.531 | −18.401 | 1.00 | 44.13 | O |
| ATOM | 4904 | N | ASP | B | 63 | 2.663 | −35.323 | −20.271 | 1.00 | 41.22 | N |
| ATOM | 4906 | CA | ASP | B | 63 | 2.815 | −36.486 | −21.145 | 1.00 | 42.30 | C |
| ATOM | 4908 | CB | ASP | B | 63 | 2.073 | −36.041 | −22.522 | 1.00 | 43.88 | C |
| ATOM | 4911 | CG | ASP | B | 63 | 0.626 | −35.635 | −22.532 | 1.00 | 43.33 | C |
| ATOM | 4912 | OD1 | ASP | B | 63 | −0.122 | −36.022 | −21.604 | 1.00 | 51.40 | O |
| ATOM | 4913 | OD2 | ASP | B | 63 | 0.165 | −34.914 | −23.426 | 1.00 | 45.91 | O |
| ATOM | 4914 | C | ASP | B | 63 | 3.792 | −37.271 | −21.232 | 1.00 | 41.93 | C |
| ATOM | 4915 | O | ASP | B | 63 | 3.785 | −38.504 | −21.282 | 1.00 | 40.53 | O |
| ATOM | 4916 | N | ARG | B | 64 | 4.882 | −36.527 | −21.131 | 1.00 | 41.22 | N |
| ATOM | 4918 | CA | ARG | B | 64 | 6.168 | −36.981 | −21.617 | 1.00 | 45.68 | C |
| ATOM | 4920 | CB | ARG | B | 64 | 6.304 | −36.552 | −23.088 | 1.00 | 47.24 | C |
| ATOM | 4923 | CG | ARG | B | 64 | 7.565 | −37.010 | −23.806 | 1.00 | 62.20 | C |
| ATOM | 4926 | CD | ARG | B | 64 | 8.577 | −35.883 | −24.017 | 1.00 | 88.15 | C |
| ATOM | 4929 | NE | ARG | B | 64 | 8.751 | −35.534 | −25.426 | 1.00 | 97.22 | N |
| ATOM | 4931 | CZ | ARG | B | 64 | 9.788 | −35.905 | −26.171 | 1.00 | 93.67 | C |
| ATOM | 4932 | NH1 | ARG | B | 64 | 10.759 | −36.650 | −25.654 | 1.00 | 83.01 | N |
| ATOM | 4935 | NH2 | ARG | B | 64 | 9.846 | −35.539 | −27.444 | 1.00 | 90.87 | N |
| ATOM | 4938 | C | ARG | B | 64 | 7.163 | −36.262 | −20.713 | 1.00 | 46.73 | C |
| ATOM | 4939 | O | ARG | B | 64 | 7.057 | −35.056 | −20.508 | 1.00 | 52.10 | O |
| ATOM | 4940 | N | ASN | B | 65 | 8.075 | −37.002 | −20.110 | 1.00 | 37.53 | N |
| ATOM | 4942 | CA | ASN | B | 65 | 9.130 | −36.401 | −19.299 | 1.00 | 40.15 | C |
| ATOM | 4944 | CB | ASN | B | 65 | 10.010 | −37.511 | −18.732 | 1.00 | 36.35 | C |
| ATOM | 4947 | CG | ASN | B | 65 | 11.071 | −37.015 | −17.754 | 1.00 | 45.31 | C |
| ATOM | 4948 | OD1 | ASN | B | 65 | 10.871 | −36.076 | −16.988 | 1.00 | 61.21 | O |
| ATOM | 4949 | ND2 | ASN | B | 65 | 12.200 | −37.703 | −17.750 | 1.00 | 46.29 | N |
| ATOM | 4952 | C | ASN | B | 65 | 9.957 | −35.381 | −20.082 | 1.00 | 35.90 | C |
| ATOM | 4953 | O | ASN | B | 65 | 10.448 | −35.705 | −21.148 | 1.00 | 46.24 | O |
| ATOM | 4954 | N | LEU | B | 66 | 10.113 | −34.165 | −19.541 | 1.00 | 36.65 | N |
| ATOM | 4956 | CA | LEU | B | 66 | 10.865 | −33.082 | −20.201 | 1.00 | 37.23 | C |
| ATOM | 4958 | CB | LEU | B | 66 | 10.032 | −31.810 | −20.397 | 1.00 | 39.56 | C |
| ATOM | 4961 | CG | LEU | B | 66 | 8.577 | −31.973 | −20.818 | 1.00 | 47.31 | C |
| ATOM | 4963 | CD1 | LEU | B | 66 | 7.931 | −30.611 | −20.961 | 1.00 | 46.56 | C |
| ATOM | 4967 | CD2 | LEU | B | 66 | 8.512 | −32.774 | −22.120 | 1.00 | 46.55 | C |
| ATOM | 4971 | C | LEU | B | 66 | 12.052 | −32.684 | −19.354 | 1.00 | 34.55 | C |
| ATOM | 4972 | O | LEU | B | 66 | 11.997 | −32.678 | −18.131 | 1.00 | 35.72 | O |
| ATOM | 4973 | N | ALA | B | 67 | 13.128 | −32.293 | −20.008 | 1.00 | 39.05 | N |
| ATOM | 4975 | CA | ALA | B | 67 | 14.246 | −31.739 | −19.265 | 1.00 | 38.84 | C |
| ATOM | 4977 | CB | ALA | B | 67 | 15.449 | −32.587 | −19.434 | 1.00 | 38.77 | C |
| ATOM | 4981 | C | ALA | B | 67 | 14.453 | −30.321 | −19.769 | 1.00 | 42.93 | C |
| ATOM | 4982 | O | ALA | B | 67 | 14.921 | −30.132 | −20.875 | 1.00 | 50.89 | O |
| ATOM | 4983 | N | MET | B | 68 | 13.989 | −29.343 | −18.988 | 1.00 | 42.23 | N |
| ATOM | 4985 | CA | MET | B | 68 | 13.936 | −27.943 | −19.405 | 1.00 | 38.93 | C |
| ATOM | 4987 | CB | MET | B | 68 | 12.661 | −27.247 | −18.910 | 1.00 | 38.21 | C |
| ATOM | 4990 | CG | MET | B | 68 | 11.378 | −27.954 | −19.341 | 1.00 | 45.18 | C |
| ATOM | 4993 | SD | MET | B | 68 | 9.909 | −26.956 | −19.043 | 1.00 | 52.21 | S |
| ATOM | 4994 | CE | MET | B | 68 | 10.056 | −26.473 | −17.377 | 1.00 | 43.00 | C |
| ATOM | 4998 | C | MET | B | 68 | 15.082 | −27.210 | −18.768 | 1.00 | 41.23 | C |
| ATOM | 4999 | O | MET | B | 68 | 15.127 | −27.064 | −17.535 | 1.00 | 35.35 | O |
| ATOM | 5000 | N | GLY | B | 69 | 15.991 | −26.724 | −19.607 | 1.00 | 40.17 | N |
| ATOM | 5002 | CA | GLY | B | 69 | 17.170 | −26.058 | −19.103 | 1.00 | 40.92 | C |
| ATOM | 5005 | C | GLY | B | 69 | 16.865 | −24.586 | −19.017 | 1.00 | 38.05 | C |
| ATOM | 5006 | O | GLY | B | 69 | 16.568 | −23.958 | −20.029 | 1.00 | 44.74 | O |
| ATOM | 5007 | N | VAL | B | 70 | 16.982 | −24.029 | −17.820 | 1.00 | 35.90 | N |
| ATOM | 5009 | CA | VAL | B | 70 | 16.570 | −22.653 | −17.546 | 1.00 | 40.11 | C |
| ATOM | 5011 | CB | VAL | B | 70 | 15.421 | −22.576 | −16.466 | 1.00 | 43.31 | C |
| ATOM | 5013 | CG1 | VAL | B | 70 | 14.647 | −21.310 | −16.669 | 1.00 | 59.11 | C |
| ATOM | 5017 | CG2 | VAL | B | 70 | 14.479 | −23.782 | −16.517 | 1.00 | 59.24 | C |
| ATOM | 5021 | C | VAL | B | 70 | 17.685 | −21.902 | −16.864 | 1.00 | 32.41 | C |
| ATOM | 5022 | O | VAL | B | 70 | 18.333 | −22.466 | −15.981 | 1.00 | 44.33 | O |
| ATOM | 5023 | N | ASN | B | 71 | 17.775 | −20.604 | −17.116 | 1.00 | 34.03 | N |
| ATOM | 5025 | CA | ASN | B | 71 | 18.514 | −19.680 | −16.259 | 1.00 | 36.90 | C |
| ATOM | 5027 | CB | ASN | B | 71 | 19.016 | −18.518 | −17.113 | 1.00 | 38.71 | C |
| ATOM | 5030 | CG | ASN | B | 71 | 19.931 | −17.594 | −16.347 | 1.00 | 46.50 | C |
| ATOM | 5031 | OD1 | ASN | B | 71 | 19.482 | −16.837 | −15.497 | 1.00 | 48.33 | O |
| ATOM | 5032 | ND2 | ASN | B | 71 | 21.234 | −17.678 | −16.622 | 1.00 | 63.32 | N |
| ATOM | 5035 | C | ASN | B | 71 | 17.669 | −19.127 | −15.109 | 1.00 | 37.70 | C |
| ATOM | 5036 | O | ASN | B | 71 | 16.739 | −18.349 | −15.311 | 1.00 | 32.10 | O |
| ATOM | 5037 | N | LEU | B | 72 | 17.994 | −19.528 | −13.891 | 1.00 | 34.55 | N |
| ATOM | 5039 | CA | LEU | B | 72 | 17.157 | −19.213 | −12.752 | 1.00 | 39.99 | C |
| ATOM | 5041 | CB | LEU | B | 72 | 17.625 | −20.008 | −11.537 | 1.00 | 37.75 | C |
| ATOM | 5044 | CG | LEU | B | 72 | 17.486 | −21.525 | −11.636 | 1.00 | 47.61 | C |
| ATOM | 5046 | CD1 | LEU | B | 72 | 17.821 | −22.170 | −10.291 | 1.00 | 38.41 | C |
| ATOM | 5050 | CD2 | LEU | B | 72 | 16.074 | −21.907 | −12.082 | 1.00 | 50.03 | C |
| ATOM | 5054 | C | LEU | B | 72 | 17.067 | −17.725 | −12.386 | 1.00 | 43.70 | C |
| ATOM | 5055 | O | LEU | B | 72 | 16.127 | −17.303 | −11.703 | 1.00 | 40.66 | O |
| ATOM | 5056 | N | THR | B | 73 | 18.029 | −16.924 | −12.815 | 1.00 | 41.25 | N |
| ATOM | 5058 | CA | THR | B | 73 | 17.959 | −15.501 | −12.514 | 1.00 | 44.38 | C |
| ATOM | 5060 | CB | THR | B | 73 | 19.372 | −14.849 | −12.718 | 1.00 | 52.96 | C |
| ATOM | 5062 | OG1 | THR | B | 73 | 20.208 | −15.133 | −11.582 | 1.00 | 54.39 | O |
| ATOM | 5064 | CG2 | THR | B | 73 | 19.290 | −13.332 | −12.699 | 1.00 | 38.83 | C |
| ATOM | 5668 | C | THR | B | 73 | 16.912 | −14.877 | −13.445 | 1.00 | 44.80 | C |
| ATOM | 5069 | O | THR | B | 73 | 16.075 | −14.049 | −13.043 | 1.00 | 37.27 | O |
| ATOM | 5070 | N | SER | B | 74 | 16.977 | −15.272 | −14.713 | 1.00 | 42.26 | N |
| ATOM | 5072 | CA | SER | B | 74 | 15.979 | −14.826 | −15.666 | 1.00 | 44.68 | C |
| ATOM | 5074 | CB | SER | B | 74 | 16.359 | −15.219 | −17.092 | 1.00 | 42.09 | C |
| ATOM | 5077 | OG | SER | B | 74 | 16.893 | −16.528 | −17.188 | 1.00 | 58.13 | O |
| ATOM | 5079 | C | SER | B | 74 | 14.547 | −15.264 | −15.309 | 1.00 | 43.90 | C |
| ATOM | 5080 | O | SER | B | 74 | 13.607 | −14.517 | −15.555 | 1.00 | 44.76 | O |
| ATOM | 5081 | N | MET | B | 75 | 14.381 | −16.447 | −14.717 | 1.00 | 34.16 | N |
| ATOM | 5083 | CA | MET | B | 75 | 13.058 | −16.934 | −14.323 | 1.00 | 32.78 | C |
| ATOM | 5085 | CB | MET | B | 75 | 13.131 | −18.476 | −14.173 | 1.00 | 32.17 | C |
| ATOM | 5088 | CG | MET | B | 75 | 11.927 | −19.172 | −13.526 | 1.00 | 33.24 | C |
| ATOM | 5091 | SD | MET | B | 75 | 11.962 | −21.000 | −13.558 | 1.00 | 39.85 | S |
| ATOM | 5092 | CE | MET | B | 75 | 10.272 | −21.290 | −13.265 | 1.00 | 46.80 | C |
| ATOM | 5096 | C | MET | B | 75 | 12.565 | −16.235 | −13.036 | 1.00 | 36.55 | C |
| ATOM | 5097 | O | MET | B | 75 | 11.354 | −16.008 | −12.854 | 1.00 | 32.68 | O |
| ATOM | 5098 | N | SER | B | 76 | 13.495 | −15.886 | −12.144 | 1.00 | 33.99 | N |
| ATOM | 5100 | CA | SER | B | 76 | 13.146 | −15.151 | −10.929 | 1.00 | 43.74 | C |
| ATOM | 5102 | CB | SER | B | 76 | 14.348 | −15.028 | −9.992 | 1.00 | 46.37 | C |
| ATOM | 5105 | OG | SER | B | 76 | 13.920 | −14.641 | −8.686 | 1.00 | 42.12 | O |
| ATOM | 5107 | C | SER | B | 76 | 12.633 | −13.748 | −11.241 | 1.00 | 45.94 | C |
| ATOM | 5108 | O | SER | B | 76 | 11.653 | −13.274 | −10.652 | 1.00 | 53.26 | O |
| ATOM | 5109 | N | LYS | B | 77 | 13.315 | −13.118 | −12.186 | 1.00 | 37.64 | N |
| ATOM | 5111 | CA | LYS | B | 77 | 12.903 | −11.850 | −12.775 | 1.00 | 46.73 | C |
| ATOM | 5113 | CB | LYS | B | 77 | 13.888 | −11.436 | −13.885 | 1.00 | 39.46 | C |
| ATOM | 5116 | CG | LYS | B | 77 | 15.252 | −11.000 | −13.319 | 1.00 | 51.78 | C |
| ATOM | 5119 | CD | LYS | B | 77 | 16.276 | −10.567 | −14.386 | 1.00 | 62.11 | C |
| ATOM | 5122 | CE | LYS | B | 77 | 17.565 | −9.995 | −13.756 | 1.00 | 63.33 | C |
| ATOM | 5125 | NZ | LYS | B | 77 | 17.408 | −8.694 | −13.023 | 1.00 | 58.81 | N |
| ATOM | 5129 | C | LYS | B | 77 | 11.472 | −11.921 | −13.305 | 1.00 | 48.48 | C |
| ATOM | 5130 | O | LYS | B | 77 | 10.595 | −11.261 | −12.750 | 1.00 | 53.70 | O |
| ATOM | 5131 | N | ILE | B | 78 | 11.237 | −12.766 | −14.307 | 1.00 | 42.93 | N |
| ATOM | 5133 | CA | ILE | B | 78 | 9.896 | −13.035 | −14.815 | 1.00 | 43.79 | C |
| ATOM | 5135 | CB | ILE | B | 78 | 9.967 | −14.212 | −15.815 | 1.00 | 41.24 | C |
| ATOM | 5137 | CG1 | ILE | B | 78 | 10.853 | −13.766 | −16.997 | 1.00 | 45.61 | C |
| ATOM | 5140 | CD1 | ILE | B | 78 | 11.105 | −14.817 | −18.082 | 1.00 | 48.24 | C |
| ATOM | 5144 | CG2 | ILE | B | 78 | 8.596 | −14.612 | −16.329 | 1.00 | 47.09 | C |
| ATOM | 5148 | C | ILE | B | 78 | 8.862 | −13.223 | −13.707 | 1.00 | 45.33 | C |
| ATOM | 5149 | O | ILE | B | 78 | 7.882 | −12.485 | −13.654 | 1.00 | 44.78 | O |
| ATOM | 5150 | N | LEU | B | 79 | 9.103 | −14.133 | −12.770 | 1.00 | 46.03 | N |
| ATOM | 5152 | CA | LEU | B | 79 | 8.147 | −14.345 | −11.682 | 1.00 | 43.74 | C |
| ATOM | 5154 | CB | LEU | B | 79 | 8.565 | −15.513 | −10.776 | 1.00 | 28.46 | C |
| ATOM | 5157 | CG | LEU | B | 79 | 8.152 | −16.859 | −11.362 | 1.00 | 44.22 | C |
| ATOM | 5159 | CD1 | LEU | B | 79 | 8.915 | −18.059 | −10.727 | 1.00 | 44.79 | C |
| ATOM | 5163 | CD2 | LEU | B | 79 | 6.660 | −17.003 | −11.221 | 1.00 | 55.35 | C |
| ATOM | 5167 | C | LEU | B | 79 | 7.998 | −13.098 | −10.819 | 1.00 | 47.18 | C |
| ATOM | 5168 | O | LEU | B | 79 | 7.050 | −12.989 | −10.059 | 1.00 | 47.68 | O |
| ATOM | 5169 | N | LYS | B | 80 | 8.954 | −12.177 | −10.904 | 1.00 | 56.91 | N |
| ATOM | 5171 | CA | LYS | B | 80 | 8.848 | −10.924 | −10.162 | 1.00 | 59.37 | C |
| ATOM | 5173 | CB | LYS | B | 80 | 10.189 | −10.193 | −10.134 | 1.00 | 57.44 | C |
| ATOM | 5176 | CG | LYS | B | 80 | 10.896 | −10.291 | −8.777 | 1.00 | 57.45 | C |
| ATOM | 5179 | CD | LYS | B | 80 | 12.393 | −9.937 | −8.861 | 1.00 | 68.50 | C |
| ATOM | 5182 | CE | LYS | B | 80 | 13.127 | −10.337 | −7.571 | 1.00 | 73.77 | C |
| ATOM | 5185 | NZ | LYS | B | 80 | 14.565 | −9.923 | −7.558 | 1.00 | 76.68 | N |
| ATOM | 5189 | C | LYS | B | 80 | 7.769 | −10.034 | −10.765 | 1.00 | 58.38 | C |
| ATOM | 5190 | O | LYS | B | 80 | 7.159 | −9.220 | −10.082 | 1.00 | 68.24 | O |
| ATOM | 5191 | N | CYS | B | 81 | 7.533 | −10.219 | −12.056 | 1.00 | 62.92 | N |
| ATOM | 5193 | CA | CYS | B | 81 | 6.450 | −9.556 | −12.779 | 1.00 | 61.81 | C |
| ATOM | 5195 | CB | CYS | B | 81 | 6.613 | −9.862 | −14.262 | 1.00 | 58.43 | C |
| ATOM | 5198 | SG | CYS | B | 81 | 8.202 | −9.251 | −14.924 | 1.00 | 60.25 | S |
| ATOM | 5199 | C | CYS | B | 81 | 5.015 | −9.874 | −12.303 | 1.00 | 63.51 | C |
| ATOM | 5200 | O | CYS | B | 81 | 4.120 | −9.045 | −12.467 | 1.00 | 70.15 | O |
| ATOM | 5201 | N | ALA | B | 82 | 4.790 | −11.026 | −11.671 | 1.00 | 60.97 | N |
| ATOM | 5203 | CA | ALA | B | 82 | 3.428 | −11.432 | −11.295 | 1.00 | 62.50 | C |
| ATOM | 5205 | CB | ALA | B | 82 | 3.325 | −12.964 | −11.240 | 1.00 | 61.96 | C |
| ATOM | 5209 | C | ALA | B | 82 | 2.917 | −10.839 | −9.977 | 1.00 | 55.92 | C |
| ATOM | 5210 | O | ALA | B | 82 | 3.658 | −10.765 | −8.994 | 1.00 | 50.05 | O |
| ATOM | 5211 | N | GLY | B | 83 | 1.630 | −10.490 | −9.948 | 1.00 | 63.59 | N |
| ATOM | 5213 | CA | GLY | B | 83 | 0.911 | −10.200 | −8.707 | 1.00 | 64.03 | C |
| ATOM | 5216 | C | GLY | B | 83 | 1.079 | −11.309 | −7.686 | 1.00 | 70.31 | C |
| ATOM | 5217 | O | GLY | B | 83 | 1.249 | −12.466 | −8.069 | 1.00 | 73.18 | O |
| ATOM | 5218 | N | ASN | B | 84 | 1.067 | −10.995 | −6.391 | 1.00 | 72.16 | N |
| ATOM | 5220 | CA | ASN | B | 84 | 1.198 | −12.070 | −5.401 | 1.00 | 69.18 | C |
| ATOM | 5222 | CB | ASN | B | 84 | 1.606 | −11.547 | −4.015 | 1.00 | 69.08 | C |
| ATOM | 5225 | CG | ASN | B | 84 | 3.109 | −11.703 | −3.736 | 1.00 | 80.96 | C |
| ATOM | 5226 | OD1 | ASN | B | 84 | 3.615 | −12.808 | −3.519 | 1.00 | 65.28 | O |
| ATOM | 5227 | ND2 | ASN | B | 84 | 3.825 | −10.578 | −3.720 | 1.00 | 88.95 | B |
| ATOM | 5230 | C | ASN | B | 84 | −0.110 | −12.853 | −5.316 | 1.00 | 59.99 | C |
| ATOM | 5231 | O | ASN | B | 84 | −0.112 | −13.962 | −4.795 | 1.00 | 67.84 | O |
| ATOM | 5232 | N | GLU | B | 85 | −1.184 | −12.278 | −5.862 | 1.00 | 51.60 | B |
| ATOM | 5234 | CA | GLU | B | 85 | −2.523 | −12.875 | −5.929 | 1.00 | 53.63 | C |
| ATOM | 5236 | CB | GLU | B | 85 | −3.579 | −11.827 | −5.541 | 1.00 | 60.59 | C |
| ATOM | 5239 | CG | GLU | B | 85 | −4.959 | −12.395 | −5.210 | 1.00 | 68.48 | C |
| ATOM | 5242 | CD | GLU | B | 85 | −5.098 | −12.816 | −3.748 | 1.00 | 90.49 | C |
| ATOM | 5243 | OE1 | GLU | B | 85 | −4.586 | −12.088 | −2.861 | 1.00 | 78.66 | O |
| ATOM | 5244 | OE2 | GLU | B | 85 | −5.714 | −13.880 | −3.485 | 1.00 | 94.77 | O |
| ATOM | 5245 | C | GLU | B | 85 | −2.842 | −13.368 | −7.340 | 1.00 | 51.12 | C |
| ATOM | 5246 | O | GLU | B | 85 | −3.985 | −13.625 | −7.711 | 1.00 | 52.97 | O |
| ATOM | 5247 | N | ASP | B | 86 | −1.817 | −13.483 | −8.159 | 1.00 | 55.56 | N |
| ATOM | 5249 | CA | ASP | B | 86 | −2.058 | −13.835 | −9.543 | 1.00 | 54.63 | C |
| ATOM | 5251 | CB | ASP | B | 86 | −0.936 | −13.265 | −10.420 | 1.00 | 58.51 | C |
| ATOM | 5254 | CG | ASP | B | 86 | −1.166 | −11.802 | −10.779 | 1.00 | 65.93 | C |
| ATOM | 5255 | OD1 | ASP | B | 86 | −1.885 | −11.078 | −10.052 | 1.00 | 70.97 | O |
| ATOM | 5256 | OD2 | ASP | B | 86 | −0.689 | −11.287 | −11.802 | 1.00 | 50.66 | O |
| ATOM | 5257 | C | ASP | B | 86 | −2.137 | −15.356 | −9.640 | 1.00 | 49.44 | C |
| ATOM | 5258 | O | ASP | B | 86 | −1.477 | −16.071 | −8.892 | 1.00 | 48.33 | O |
| ATOM | 5259 | N | ILE | B | 87 | −3.002 | −15.818 | −10.530 | 1.00 | 47.18 | N |
| ATOM | 5261 | CA | ILE | B | 87 | −3.055 | −17.210 | −10.949 | 1.00 | 45.65 | C |
| ATOM | 5263 | CB | ILE | B | 87 | −4.440 | −17.463 | −11.573 | 1.00 | 38.87 | C |
| ATOM | 5265 | CG1 | ILE | B | 87 | −5.529 | −17.383 | −10.495 | 1.00 | 46.51 | C |
| ATOM | 5268 | CD1 | ILE | B | 87 | −6.912 | −17.197 | −11.107 | 1.00 | 37.82 | C |
| ATOM | 5272 | CG2 | ILE | B | 87 | −4.494 | −18.737 | −12.386 | 1.00 | 37.50 | C |
| ATOM | 5276 | C | ILE | B | 87 | −1.960 | −17.455 | −11.984 | 1.00 | 41.15 | C |
| ATOM | 5277 | O | ILE | B | 87 | −1.960 | −16.841 | −13.057 | 1.00 | 36.29 | O |
| ATOM | 5278 | N | ILE | B | 88 | −1.083 | −18.407 | −11.682 | 1.00 | 34.04 | N |
| ATOM | 5280 | CA | ILE | B | 88 | 0.038 | −18.721 | −12.557 | 1.00 | 39.16 | C |
| ATOM | 5282 | CB | ILE | B | 88 | 1.316 | −18.828 | −11.722 | 1.00 | 35.81 | C |
| ATOM | 5284 | CG1 | ILE | B | 88 | 1.480 | −17.560 | −10.873 | 1.00 | 46.46 | C |
| ATOM | 5287 | CD1 | ILE | B | 88 | 2.867 | −16.939 | −10.957 | 1.00 | 58.64 | C |
| ATOM | 5291 | CG2 | ILE | B | 88 | 2.510 | −19.072 | −12.629 | 1.00 | 34.09 | C |
| ATOM | 5295 | C | ILE | B | 88 | −0.176 | −20.013 | −13.327 | 1.00 | 40.12 | C |
| ATOM | 5296 | O | ILE | B | 88 | −0.530 | −21.041 | −12.770 | 1.00 | 46.92 | O |
| ATOM | 5297 | N | THR | B | 89 | 0.175 | −20.001 | −14.597 | 1.00 | 41.52 | N |
| ATOM | 5299 | CA | THR | B | 89 | 0.135 | −21.216 | −15.378 | 1.00 | 37.89 | C |
| ATOM | 5301 | CB | THR | B | 89 | −0.903 | −21.014 | −16.483 | 1.00 | 34.02 | C |
| ATOM | 5303 | OG1 | THR | B | 89 | −2.172 | −20.663 | −15.913 | 1.00 | 41.04 | O |
| ATOM | 5305 | CG2 | THR | B | 89 | −1.127 | −22.296 | −17.216 | 1.00 | 32.93 | C |
| ATOM | 5309 | C | THR | B | 89 | 1.517 | −21.495 | −15.973 | 1.00 | 34.30 | C |
| ATOM | 5310 | O | THR | B | 89 | 2.067 | −20.658 | −16.694 | 1.00 | 33.57 | O |
| ATOM | 5311 | N | LEU | B | 90 | 2.077 | −22.674 | −15.704 | 1.00 | 38.33 | N |
| ATOM | 5313 | CA | LEU | B | 90 | 3.310 | −23.053 | −16.388 | 1.00 | 33.34 | C |
| ATOM | 5315 | CB | LEU | B | 90 | 4.282 | −23.759 | −15.465 | 1.00 | 37.17 | C |
| ATOM | 5318 | CG | LEU | B | 90 | 4.696 | −23.055 | −14.172 | 1.00 | 30.88 | C |
| ATOM | 5320 | CD1 | LEU | B | 90 | 5.811 | −23.873 | −13.497 | 1.00 | 41.66 | C |
| ATOM | 5324 | CD2 | LEU | B | 90 | 5.198 | −21.657 | −14.454 | 1.00 | 52.34 | C |
| ATOM | 5328 | C | LEU | B | 90 | 2.928 | −23.933 | −17.560 | 1.00 | 38.35 | C |
| ATOM | 5329 | O | LEU | B | 90 | 2.110 | −24.840 | −17.428 | 1.00 | 39.98 | O |
| ATOM | 5330 | N | ARG | B | 91 | 3.474 | −23.649 | −18.730 | 1.00 | 41.50 | N |
| ATOM | 5332 | CA | ARG | B | 91 | 3.165 | −24.488 | −19.873 | 1.00 | 37.33 | C |
| ATOM | 5334 | CB | ARG | B | 91 | 2.082 | −23.812 | −20.701 | 1.00 | 38.14 | C |
| ATOM | 5337 | CG | ARG | B | 91 | 1.431 | −24.756 | −21.667 | 1.00 | 39.24 | C |
| ATOM | 5340 | CD | ARG | B | 91 | 0.489 | −24.113 | −22.687 | 1.00 | 49.53 | C |
| ATOM | 5343 | NE | ARG | B | 91 | −0.067 | −22.832 | −22.259 | 1.00 | 62.88 | N |
| ATOM | 5345 | CZ | ARG | B | 91 | −1.213 | −22.700 | −21.609 | 1.00 | 75.00 | C |
| ATOM | 5346 | NH1 | ARG | B | 91 | −1.918 | −23.783 | −21.290 | 1.00 | 82.35 | N |
| ATOM | 5349 | NH2 | ARG | B | 91 | −1.646 | −21.490 | −21.266 | 1.00 | 61.41 | N |
| ATOM | 5352 | C | ARG | B | 91 | 4.389 | −24.710 | −20.753 | 1.00 | 43.64 | C |
| ATOM | 5353 | O | ARG | B | 91 | 5.092 | −23.749 | −21.107 | 1.00 | 37.59 | O |
| ATOM | 5354 | N | ALA | B | 92 | 4.567 | −25.945 | −21.221 | 1.00 | 37.99 | N |
| ATOM | 5356 | CA | ALA | B | 92 | 5.575 | −26.202 | −22.255 | 1.00 | 49.37 | C |
| ATOM | 5358 | CB | ALA | B | 92 | 6.878 | −26.621 | −21.600 | 1.00 | 47.62 | C |
| ATOM | 5362 | C | ALA | B | 92 | 5.136 | −27.261 | −23.265 | 1.00 | 55.64 | C |
| ATOM | 5363 | O | ALA | B | 92 | 4.502 | −28.222 | −22.879 | 1.00 | 46.41 | O |
| ATOM | 5364 | N | GLU | B | 93 | 5.494 | −27.147 | −24.544 | 1.00 | 70.00 | N |
| ATOM | 5366 | CA | GLU | B | 93 | 4.994 | −28.135 | −25.519 | 1.00 | 80.82 | C |
| ATOM | 5368 | CB | GLU | B | 93 | 4.727 | −27.540 | −26.910 | 1.00 | 81.50 | C |
| ATOM | 5371 | CG | GLU | B | 93 | 3.387 | −27.972 | −27.516 | 1.00 | 87.61 | C |
| ATOM | 5374 | CD | GLU | B | 93 | 3.445 | −29.270 | −28.329 | 1.00 | 92.21 | C |
| ATOM | 5375 | OE1 | GLU | B | 93 | 3.285 | −29.188 | −29.568 | 1.00 | 90.80 | O |
| ATOM | 5376 | OE2 | GLU | B | 93 | 3.609 | −30.379 | −27.751 | 1.00 | 75.27 | O |
| ATOM | 5377 | C | GLU | B | 93 | 5.811 | −29.430 | −25.630 | 1.00 | 87.61 | C |
| ATOM | 5378 | O | GLU | B | 93 | 5.241 | −30.530 | −25.619 | 1.00 | 94.38 | O |
| ATOM | 5379 | N | ASP | B | 94 | 7.130 | −29.310 | −25.760 | 1.00 | 91.99 | N |
| ATOM | 5381 | CA | ASP | B | 94 | 8.031 | −30.464 | −25.674 | 1.00 | 95.02 | C |
| ATOM | 5383 | CB | ASP | B | 94 | 7.424 | −31.746 | −26.271 | 1.00 | 93.37 | C |
| ATOM | 5386 | CG | ASP | B | 94 | 7.097 | −31.620 | −27.756 | 1.00 | 103.76 | C |
| ATOM | 5387 | OD1 | ASP | B | 94 | 7.598 | −30.680 | −28.418 | 1.00 | 114.43 | O |
| ATOM | 5388 | OD2 | ASP | B | 94 | 6.348 | −32.426 | −28.355 | 1.00 | 110.54 | O |
| ATOM | 5389 | C | ASP | B | 94 | 9.383 | −30.148 | −26.310 | 1.00 | 94.92 | C |
| ATOM | 5390 | O | ASP | B | 94 | 9.647 | −30.503 | −27.467 | 1.00 | 83.22 | O |
| ATOM | 5391 | N | ASN | B | 95 | 10.198 | −29.423 | −25.539 | 1.00 | 97.42 | N |
| ATOM | 5393 | CA | ASN | B | 95 | 11.645 | −29.313 | −25.754 | 1.00 | 98.50 | C |
| ATOM | 5395 | CB | ASN | B | 95 | 12.168 | −30.358 | −26.762 | 1.00 | 98.14 | C |
| ATOM | 5398 | CG | ASN | B | 95 | 12.482 | −31.708 | −26.112 | 1.00 | 97.84 | C |
| ATOM | 5399 | OD1 | ASN | B | 95 | 12.333 | −31.879 | −24.898 | 1.00 | 87.40 | O |
| ATOM | 5400 | ND2 | ASN | B | 95 | 12.922 | −32.672 | −26.928 | 1.00 | 94.88 | N |
| ATOM | 5403 | C | ASN | B | 95 | 12.040 | −27.887 | −26.159 | 1.00 | 96.55 | C |
| ATOM | 5404 | O | ASN | B | 95 | 11.631 | −26.921 | −25.506 | 1.00 | 88.91 | O |
| ATOM | 5405 | N | ALA | B | 96 | 12.847 | −27.762 | −27.214 | 1.00 | 93.80 | N |
| ATOM | 5407 | CA | ALA | B | 96 | 13.321 | −26.458 | −27.667 | 1.00 | 87.76 | C |
| ATOM | 5409 | CB | ALA | B | 96 | 13.390 | −26.423 | −29.198 | 1.00 | 86.50 | C |
| ATOM | 5413 | C | ALA | B | 96 | 12.378 | −25.374 | −27.138 | 1.00 | 81.01 | C |
| ATOM | 5414 | O | ALA | B | 96 | 11.169 | −25.457 | −27.342 | 1.00 | 78.19 | O |
| ATOM | 5415 | N | ASP | B | 97 | 12.932 | −24.422 | −26.386 | 1.00 | 74.88 | N |
| ATOM | 5417 | CA | ASP | B | 97 | 12.299 | −23.132 | −26.112 | 1.00 | 67.25 | C |
| ATOM | 5419 | CB | ASP | B | 97 | 12.238 | −22.254 | −27.379 | 1.00 | 75.54 | C |
| ATOM | 5422 | CG | ASP | B | 97 | 11.230 | −22.756 | −28.425 | 1.00 | 91.72 | C |
| ATOM | 5423 | OD1 | ASP | B | 97 | 11.672 | −23.347 | −29.439 | 1.00 | 103.07 | O |
| ATOM | 5424 | OD2 | ASP | B | 97 | 9.992 | −22.553 | −28.362 | 1.00 | 93.01 | O |
| ATOM | 5425 | C | ASP | B | 97 | 10.929 | −23.257 | −25.464 | 1.00 | 61.44 | C |
| ATOM | 5426 | O | ASP | B | 97 | 10.557 | −24.319 | −25.001 | 1.00 | 55.85 | O |
| ATOM | 5427 | N | THR | B | 98 | 10.166 | −22.174 | −25.436 | 1.00 | 58.62 | N |
| ATOM | 5429 | CA | THR | B | 98 | 8.987 | −22.132 | −24.597 | 1.00 | 55.46 | C |
| ATOM | 5431 | CB | THR | B | 98 | 7.693 | −22.473 | −25.405 | 1.00 | 60.34 | C |
| ATOM | 5433 | OG1 | THR | B | 98 | 7.582 | −23.892 | −25.653 | 1.00 | 66.39 | O |
| ATOM | 5435 | CG2 | THR | B | 98 | 7.683 | −21.803 | −26.787 | 1.00 | 75.10 | C |
| ATOM | 5439 | C | THR | B | 98 | 9.055 | −23.004 | −23.320 | 1.00 | 44.54 | C |
| ATOM | 5440 | O | THR | B | 98 | 8.679 | −24.154 | −23.318 | 1.00 | 40.65 | O |
| ATOM | 5441 | N | LEU | B | 99 | 9.364 | −22.394 | −22.189 | 1.00 | 38.12 | N |
| ATOM | 5443 | CA | LEU | B | 99 | 8.473 | −22.502 | −21.050 | 1.00 | 37.04 | C |
| ATOM | 5445 | CB | LEU | B | 99 | 9.305 | −22.715 | −19.801 | 1.00 | 28.43 | C |
| ATOM | 5448 | CG | LEU | B | 99 | 8.555 | −22.677 | −18.457 | 1.00 | 42.89 | C |
| ATOM | 5450 | CD1 | LEU | B | 99 | 7.650 | −23.872 | −18.209 | 1.00 | 32.71 | C |
| ATOM | 5454 | CD2 | LEU | B | 99 | 9.504 | −22.533 | −17.285 | 1.00 | 39.84 | C |
| ATOM | 5458 | C | LEU | B | 99 | 7.600 | −21.223 | −20.920 | 1.00 | 42.99 | C |
| ATOM | 5459 | O | LEU | B | 99 | 8.117 | −20.131 | −20.683 | 1.00 | 53.80 | O |
| ATOM | 5460 | N | ALA | B | 100 | 6.286 | −21.347 | −21.079 | 1.00 | 41.66 | N |
| ATOM | 5462 | CA | ALA | B | 100 | 5.389 | −20.210 | −20.912 | 1.00 | 42.70 | C |
| ATOM | 5464 | CB | ALA | B | 100 | 4.165 | −20.368 | −21.814 | 1.00 | 44.35 | C |
| ATOM | 5468 | C | ALA | B | 100 | 4.988 | −20.022 | −19.440 | 1.00 | 46.10 | C |
| ATOM | 5469 | O | ALA | B | 100 | 4.666 | −20.998 | −18.741 | 1.00 | 39.17 | O |
| ATOM | 5470 | N | LEU | B | 101 | 5.084 | −18.777 | −18.959 | 1.00 | 42.97 | N |
| ATOM | 5472 | CA | LEU | B | 101 | 4.553 | −18.400 | −17.648 | 1.00 | 41.86 | C |
| ATOM | 5474 | CB | LEU | B | 101 | 5.664 | −17.931 | −16.712 | 1.00 | 29.84 | C |
| ATOM | 5477 | CG | LEU | B | 101 | 6.819 | −18.929 | −16.603 | 1.00 | 53.62 | C |
| ATOM | 5479 | CD1 | LEU | B | 101 | 7.932 | −18.640 | −17.641 | 1.00 | 42.56 | C |
| ATOM | 5483 | CD2 | LEU | B | 101 | 7.390 | −18.963 | −15.191 | 1.00 | 36.41 | C |
| ATOM | 5487 | C | LEU | B | 101 | 3.449 | −17.345 | −17.775 | 1.00 | 46.39 | C |
| ATOM | 5488 | O | LEU | B | 101 | 3.714 | −16.196 | −18.149 | 1.00 | 51.63 | O |
| ATOM | 5489 | N | VAL | B | 102 | 2.220 | −17.741 | −17.423 | 1.00 | 48.57 | N |
| ATOM | 5491 | CA | VAL | B | 102 | 1.048 | −16.863 | −17.492 | 1.00 | 38.86 | C |
| ATOM | 5493 | CB | VAL | B | 102 | −0.069 | −17.494 | −18.330 | 1.00 | 44.86 | C |
| ATOM | 5495 | CG1 | VAL | B | 102 | −1.240 | −16.518 | −18.568 | 1.00 | 38.55 | C |
| ATOM | 5499 | CG2 | VAL | B | 102 | 0.494 | −17.933 | −19.674 | 1.00 | 36.64 | C |
| ATOM | 5503 | C | VAL | B | 102 | 0.535 | −16.396 | −16.125 | 1.00 | 41.50 | C |
| ATOM | 5504 | O | VAL | B | 102 | 0.272 | −17.201 | −15.240 | 1.00 | 35.10 | O |
| ATOM | 5505 | N | PHE | B | 103 | 0.501 | −15.072 | −15.945 | 1.00 | 40.04 | N |
| ATOM | 5507 | CA | PHE | B | 103 | −0.007 | −14.419 | −14.756 | 1.00 | 31.62 | C |
| ATOM | 5509 | CB | PHE | B | 103 | 1.024 | −13.429 | −14.241 | 1.00 | 34.95 | C |
| ATOM | 5512 | CG | PHE | B | 103 | 2.455 | −13.963 | −14.258 | 1.00 | 41.30 | C |
| ATOM | 5513 | CD1 | PHE | B | 103 | 2.745 | −15.281 | −13.954 | 1.00 | 46.17 | C |
| ATOM | 5515 | CE1 | PHE | B | 103 | 4.085 | −15.742 | −13.923 | 1.00 | 36.92 | C |
| ATOM | 5517 | CZ | PHE | B | 103 | 5.119 | −14.888 | −14.284 | 1.00 | 38.23 | C |
| ATOM | 5519 | CE2 | PHE | B | 103 | 4.831 | −13.594 | −14.646 | 1.00 | 43.77 | C |
| ATOM | 5521 | CD2 | PHE | B | 103 | 3.515 | −13.122 | −14.612 | 1.00 | 37.68 | C |
| ATOM | 5523 | C | PHE | B | 103 | −1.332 | −13.723 | −15.027 | 1.00 | 36.89 | C |
| ATOM | 5524 | O | PHE | B | 103 | −1.409 | −12.825 | −15.870 | 1.00 | 37.18 | O |
| ATOM | 5525 | N | GLU | B | 104 | −2.412 | −14.218 | −14.411 | 1.00 | 39.00 | N |
| ATOM | 5527 | CA | GLU | B | 104 | −3.723 | −13.568 | −14.569 | 1.00 | 41.23 | C |
| ATOM | 5529 | CB | GLU | B | 104 | −4.816 | −14.513 | −15.013 | 1.00 | 38.48 | C |
| ATOM | 5532 | CG | GLU | B | 104 | −4.457 | −15.418 | −16.160 | 1.00 | 42.56 | C |
| ATOM | 5535 | CD | GLU | B | 104 | −5.418 | −16.600 | −16.212 | 1.00 | 71.10 | C |
| ATOM | 5536 | OE1 | GLU | B | 104 | −6.578 | −16.411 | −15.768 | 1.00 | 68.63 | O |
| ATOM | 5537 | OE2 | GLU | B | 104 | −5.024 | −17.704 | −16.664 | 1.00 | 60.79 | O |
| ATOM | 5538 | C | GLU | B | 104 | −4.164 | −12.960 | −13.271 | 1.00 | 37.24 | C |
| ATOM | 5539 | O | GLU | B | 104 | −4.388 | −13.661 | −12.287 | 1.00 | 36.22 | O |
| ATOM | 5540 | N | ALA | B | 105 | −4.223 | −11.643 | −13.271 | 1.00 | 41.03 | N |
| ATOM | 5542 | CA | ALA | B | 105 | −4.626 | −10.912 | −12.094 | 1.00 | 51.47 | C |
| ATOM | 5544 | CB | ALA | B | 105 | −4.471 | −9.401 | −12.338 | 1.00 | 54.62 | C |
| ATOM | 5548 | C | ALA | B | 105 | −6.071 | −11.268 | −11.746 | 1.00 | 54.11 | C |
| ATOM | 5549 | O | ALA | B | 105 | −6.883 | −11.668 | −12.607 | 1.00 | 39.69 | O |
| ATOM | 5550 | N | PRO | B | 106 | −6.364 | −11.119 | −10.460 | 1.00 | 61.37 | N |
| ATOM | 5551 | CA | PRO | B | 106 | −7.517 | −11.771 | −9.833 | 1.00 | 73.10 | C |
| ATOM | 5553 | CB | PRO | B | 106 | −7.555 | −11.118 | −8.447 | 1.00 | 76.71 | C |
| ATOM | 5556 | CG | PRO | B | 106 | −6.092 | −10.791 | −8.173 | 1.00 | 69.89 | C |
| ATOM | 5559 | CD | PRO | B | 106 | −5.588 | −10.319 | −9.499 | 1.00 | 58.30 | C |
| ATOM | 5562 | C | PRO | B | 106 | −8.837 | −11.595 | −10.593 | 1.00 | 77.81 | C |
| ATOM | 5563 | O | PRO | B | 106 | −9.460 | −12.623 | −10.869 | 1.00 | 76.23 | O |
| ATOM | 5564 | N | ASN | B | 107 | −9.204 | −10.363 | −10.969 | 1.00 | 77.70 | N |
| ATOM | 5566 | CA | ASN | B | 107 | −10.511 | −10.080 | −11.568 | 1.00 | 80.04 | C |
| ATOM | 5568 | CB | ASN | B | 107 | −11.279 | −9.047 | −10.740 | 1.00 | 82.35 | C |
| ATOM | 5571 | CG | ASN | B | 107 | −10.370 | −8.224 | −9.853 | 1.00 | 98.02 | C |
| ATOM | 5572 | OD1 | ASN | B | 107 | −9.838 | −7.196 | −10.280 | 1.00 | 105.16 | O |
| ATOM | 5573 | ND2 | ASN | B | 107 | −10.173 | −8.677 | −8.615 | 1.00 | 101.92 | N |
| ATOM | 5576 | C | ASN | B | 107 | −10.485 | −9.621 | −13.024 | 1.00 | 78.61 | C |
| ATOM | 5577 | O | ASN | B | 107 | −11.389 | −8.916 | −13.476 | 1.00 | 70.63 | O |
| ATOM | 5578 | N | GLN | B | 108 | −9.459 | −10.036 | −13.762 | 1.00 | 79.56 | N |
| ATOM | 5580 | CA | GLN | B | 108 | −9.518 | −10.077 | −15.228 | 1.00 | 77.15 | C |
| ATOM | 5582 | CB | GLN | B | 108 | −10.913 | −10.528 | −15.696 | 1.00 | 81.55 | C |
| ATOM | 5585 | CG | GLN | B | 108 | −11.612 | −11.593 | −14.824 | 1.00 | 90.15 | C |
| ATOM | 5588 | CD | GLN | B | 108 | −10.945 | −12.967 | −14.879 | 1.00 | 103.94 | C |
| ATOM | 5589 | OE1 | GLN | B | 108 | −11.380 | −13.912 | −14.197 | 1.00 | 109.01 | O |
| ATOM | 5590 | NE2 | GLN | B | 108 | −9.887 | −13.079 | −15.680 | 1.00 | 97.05 | N |
| ATOM | 5593 | C | GLN | B | 108 | −9.082 | −8.774 | −15.934 | 1.00 | 66.35 | C |
| ATOM | 5594 | O | GLN | B | 108 | −9.234 | −8.633 | −17.147 | 1.00 | 63.95 | O |
| ATOM | 5595 | N | GLU | B | 109 | −8.484 | −7.854 | −15.184 | 1.00 | 57.69 | N |
| ATOM | 5597 | CA | GLU | B | 109 | −7.962 | −6.603 | −15.734 | 1.00 | 58.47 | C |
| ATOM | 5599 | CB | GLU | B | 109 | −7.616 | −5.628 | −14.610 | 1.00 | 62.09 | C |
| ATOM | 5602 | CG | GLU | B | 109 | −6.871 | −6.235 | −13.432 | 1.00 | 74.08 | C |
| ATOM | 5605 | CD | GLU | B | 109 | −7.770 | −6.407 | −12.212 | 1.00 | 93.89 | C |
| ATOM | 5606 | OE1 | GLU | B | 109 | −8.630 | −5.521 | −11.993 | 1.00 | 95.14 | O |
| ATOM | 5607 | OE2 | GLU | B | 109 | −7.628 | −7.417 | −11.475 | 1.00 | 73.68 | O |
| ATOM | 5608 | C | GLU | B | 109 | −6.702 | −6.755 | −16.591 | 1.00 | 53.77 | C |
| ATOM | 5609 | O | GLU | B | 109 | −6.360 | −5.872 | −17.385 | 1.00 | 40.99 | O |
| ATOM | 5610 | N | LYS | B | 110 | −5.984 | −7.854 | −16.397 | 1.00 | 42.17 | N |
| ATOM | 5612 | CA | LYS | B | 110 | −4.614 | −7.885 | −16.830 | 1.00 | 42.57 | C |
| ATOM | 5614 | CB | LYS | B | 110 | −3.732 | −7.048 | −15.878 | 1.00 | 35.23 | C |
| ATOM | 5617 | CG | LYS | B | 110 | −2.298 | −6.860 | −16.360 | 1.00 | 33.08 | C |
| ATOM | 5620 | CD | LYS | B | 110 | −1.344 | −6.519 | −15.212 | 1.00 | 41.15 | C |
| ATOM | 5623 | CE | LYS | B | 110 | −0.076 | −5.872 | −15.762 | 1.00 | 49.41 | C |
| ATOM | 5626 | NZ | LYS | B | 110 | 0.869 | −5.455 | −14.693 | 1.00 | 50.94 | N |
| ATOM | 5630 | C | LYS | B | 110 | −4.158 | −9.332 | −16.898 | 1.00 | 41.81 | C |
| ATOM | 5631 | O | LYS | B | 110 | −4.334 | −10.099 | −15.954 | 1.00 | 42.26 | O |
| ATOM | 5632 | N | VAL | B | 111 | −3.532 | −9.642 | −18.026 | 1.00 | 33.82 | N |
| ATOM | 5634 | CA | VAL | B | 111 | −2.951 | −10.936 | −18.312 | 1.00 | 40.46 | C |
| ATOM | 5636 | CB | VAL | B | 111 | −3.787 | −11.654 | −19.396 | 1.00 | 40.85 | C |
| ATOM | 5638 | CG1 | VAL | B | 111 | −3.174 | −13.003 | −19.675 | 1.00 | 41.23 | C |
| ATOM | 5642 | CG2 | VAL | B | 111 | −5.197 | −11.840 | −18.871 | 1.00 | 41.50 | C |
| ATOM | 5646 | C | VAL | B | 111 | −1.558 | −10.749 | −18.877 | 1.00 | 41.03 | C |
| ATOM | 5647 | O | VAL | B | 111 | −1.358 | −9.940 | −19.774 | 1.00 | 41.65 | O |
| ATOM | 5648 | N | SER | B | 112 | −0.619 | −11.548 | −18.390 | 1.00 | 45.09 | N |
| ATOM | 5650 | CA | SER | B | 112 | 0.772 | −11.449 | −18.779 | 1.00 | 40.89 | C |
| ATOM | 5652 | CB | SER | B | 112 | 1.556 | −11.023 | −17.540 | 1.00 | 36.60 | C |
| ATOM | 5655 | OG | SER | B | 112 | 1.311 | −9.660 | −17.273 | 1.00 | 35.14 | O |
| ATOM | 5657 | C | SER | B | 112 | 1.284 | −12.797 | −19.309 | 1.00 | 36.53 | C |
| ATOM | 5658 | O | SER | B | 112 | 1.378 | −13.768 | −18.554 | 1.00 | 38.06 | O |
| ATOM | 5659 | N | ASP | B | 113 | 1.674 | −12.825 | −20.582 | 1.00 | 35.96 | N |
| ATOM | 5661 | CA | ASP | B | 113 | 2.119 | −14.051 | −21.237 | 1.00 | 38.97 | C |
| ATOM | 5663 | CB | ASP | B | 113 | 1.329 | −14.309 | −22.539 | 1.00 | 42.40 | C |
| ATOM | 5666 | CG | ASP | B | 113 | 1.719 | −15.628 | −23.233 | 1.00 | 54.12 | C |
| ATOM | 5667 | OD1 | ASP | B | 113 | 2.563 | −16.396 | −22.699 | 1.00 | 62.47 | O |
| ATOM | 5668 | OD2 | ASP | B | 113 | 1.223 | −15.988 | −24.329 | 1.00 | 86.01 | O |
| ATOM | 5669 | C | ASP | B | 113 | 3.624 | −13.916 | −21.504 | 1.00 | 39.35 | C |
| ATOM | 5670 | O | ASP | B | 113 | 4.066 | −13.137 | −22.349 | 1.00 | 41.34 | O |
| ATOM | 5671 | N | TYR | B | 114 | 4.415 | −14.671 | −20.758 | 1.00 | 32.31 | N |
| ATOM | 5673 | CA | TYR | B | 114 | 5.847 | −14.685 | −20.965 | 1.00 | 33.31 | C |
| ATOM | 5675 | CB | TYR | B | 114 | 6.628 | −14.477 | −19.682 | 1.00 | 20.88 | C |
| ATOM | 5678 | CG | TYR | B | 114 | 6.496 | −13.094 | −19.151 | 1.00 | 34.42 | C |
| ATOM | 5679 | CD1 | TYR | B | 114 | 5.532 | −12.773 | −18.186 | 1.00 | 38.21 | C |
| ATOM | 5681 | CE1 | TYR | B | 114 | 5.382 | −11.485 | −17.738 | 1.00 | 43.65 | C |
| ATOM | 5683 | CZ | TYR | B | 114 | 6.189 | −10.494 | −18.271 | 1.00 | 37.43 | C |
| ATOM | 5684 | OH | TYR | B | 114 | 6.088 | −9.205 | −17.839 | 1.00 | 43.94 | O |
| ATOM | 5686 | CE2 | TYR | B | 114 | 7.172 | −10.803 | −19.166 | 1.00 | 37.97 | C |
| ATOM | 5688 | CD2 | TYR | B | 114 | 7.302 | −12.092 | −19.627 | 1.00 | 41.23 | C |
| ATOM | 5690 | C | TYR | B | 114 | 6.320 | −15.990 | −21.543 | 1.00 | 43.86 | C |
| ATOM | 5691 | O | TYR | B | 114 | 5.648 | −17.009 | −21.429 | 1.00 | 37.97 | O |
| ATOM | 5692 | N | GLU | B | 115 | 7.482 | −15.945 | −22.198 | 1.00 | 39.71 | N |
| ATOM | 5694 | CA | GLU | B | 115 | 7.955 | −17.138 | −22.873 | 1.00 | 38.23 | C |
| ATOM | 5696 | CB | GLU | B | 115 | 7.463 | −17.146 | −24.321 | 1.00 | 40.65 | C |
| ATOM | 5699 | CG | GLU | B | 115 | 6.894 | −18.461 | −24.841 | 1.00 | 55.57 | C |
| ATOM | 5702 | CD | GLU | B | 115 | 6.332 | −18.371 | −26.257 | 1.00 | 69.30 | C |
| ATOM | 5703 | OE1 | GLU | B | 115 | 5.669 | −19.338 | −26.701 | 1.00 | 87.80 | O |
| ATOM | 5704 | OE2 | GLU | B | 115 | 6.528 | −17.333 | −26.928 | 1.00 | 57.19 | O |
| ATOM | 5705 | C | GLU | B | 115 | 9.462 | −17.163 | −22.782 | 1.00 | 34.14 | C |
| ATOM | 5706 | O | GLU | B | 115 | 10.136 | −16.217 | −23.216 | 1.00 | 39.91 | O |
| ATOM | 5707 | N | MET | B | 116 | 9.983 | −18.223 | −22.167 | 1.00 | 33.85 | N |
| ATOM | 5709 | CA | MET | B | 116 | 11.396 | −18.277 | −21.816 | 1.00 | 40.98 | C |
| ATOM | 5711 | CB | MET | B | 116 | 11.520 | −18.628 | −20.344 | 1.00 | 40.04 | C |
| ATOM | 5714 | CG | MET | B | 116 | 12.905 | −18.523 | −19.793 | 1.00 | 55.33 | C |
| ATOM | 5717 | SD | MET | B | 116 | 12.919 | −18.338 | −17.972 | 1.00 | 66.41 | S |
| ATOM | 5718 | CE | MET | B | 116 | 11.359 | −18.989 | −17.483 | 1.00 | 64.65 | C |
| ATOM | 5722 | C | MET | B | 116 | 12.104 | −19.300 | −22.700 | 1.00 | 39.59 | C |
| ATOM | 5723 | O | MET | B | 116 | 11.611 | −20.408 | −22.866 | 1.00 | 41.84 | O |
| ATOM | 5724 | N | LYS | B | 117 | 13.190 | −18.905 | −23.353 | 1.00 | 40.19 | N |
| ATOM | 5726 | CA | LYS | B | 117 | 13.928 | −19.852 | −24.201 | 1.00 | 37.38 | C |
| ATOM | 5728 | CB | LYS | B | 117 | 15.057 | −19.189 | −24.977 | 1.00 | 36.88 | C |
| ATOM | 5731 | CG | LYS | B | 117 | 14.628 | −18.544 | −26.268 | 1.00 | 58.53 | C |
| ATOM | 5734 | CD | LYS | B | 117 | 15.841 | −18.143 | −27.081 | 1.00 | 60.67 | C |
| ATOM | 5737 | CE | LYS | B | 117 | 15.401 | −17.418 | −28.343 | 1.00 | 70.45 | C |
| ATOM | 5740 | NZ | LYS | B | 117 | 14.985 | −16.018 | −28.080 | 1.00 | 50.84 | N |
| ATOM | 5744 | C | LYS | B | 117 | 14.586 | −20.785 | −23.258 | 1.00 | 34.87 | C |
| ATOM | 5745 | O | LYS | B | 117 | 15.225 | −20.331 | −22.314 | 1.00 | 38.78 | O |
| ATOM | 5746 | N | LEU | B | 118 | 14.456 | −22.072 | −23.548 | 1.00 | 36.69 | N |
| ATOM | 5748 | CA | LEU | B | 118 | 15.146 | −23.120 | −22.817 | 1.00 | 39.26 | C |
| ATOM | 5750 | CB | LEU | B | 118 | 14.218 | −24.313 | −22.666 | 1.00 | 39.85 | C |
| ATOM | 5753 | CG | LEU | B | 118 | 12.862 | −24.025 | −22.023 | 1.00 | 30.34 | O |
| ATOM | 5755 | CD1 | LEU | B | 118 | 12.007 | −25.249 | −22.200 | 1.00 | 43.19 | C |
| ATOM | 5759 | CD2 | LEU | B | 118 | 13.042 | −23.748 | −20.525 | 1.00 | 33.95 | C |
| ATOM | 5763 | C | LEU | B | 118 | 16.412 | −23.547 | −23.557 | 1.00 | 49.94 | C |
| ATOM | 5764 | O | LEU | B | 118 | 16.404 | −23.677 | −24.779 | 1.00 | 56.05 | O |
| ATOM | 5765 | N | MET | B | 119 | 17.512 | −23.730 | −22.831 | 1.00 | 58.18 | N |
| ATOM | 5767 | CA | MET | B | 119 | 18.725 | −24.268 | −23.437 | 1.00 | 63.26 | C |
| ATOM | 5769 | CB | MET | B | 119 | 19.972 | −23.607 | −22.858 | 1.00 | 63.49 | C |
| ATOM | 5772 | CG | MET | B | 119 | 20.000 | −23.560 | −21.356 | 1.00 | 70.01 | C |
| ATOM | 5775 | SD | MET | B | 119 | 20.155 | −21.900 | −20.656 | 1.00 | 82.25 | S |
| ATOM | 5776 | CE | MET | B | 119 | 19.000 | −20.895 | −21.702 | 1.00 | 86.74 | C |
| ATOM | 5780 | C | MET | B | 119 | 18.747 | −25.770 | −23.217 | 1.00 | 65.13 | C |
| ATOM | 5781 | O | MET | B | 119 | 17.958 | −26.269 | −22.427 | 1.00 | 58.55 | O |
| ATOM | 5782 | N | ASP | B | 120 | 19.569 | −26.515 | −23.954 | 1.00 | 73.46 | N |
| ATOM | 5784 | CA | ASP | B | 120 | 19.573 | −27.967 | −23.757 | 1.00 | 79.67 | C |
| ATOM | 5786 | CB | ASP | B | 120 | 19.660 | −28.772 | −25.058 | 1.00 | 84.11 | C |
| ATOM | 5789 | CG | ASP | B | 120 | 19.091 | −30.188 | −24.900 | 1.00 | 98.77 | C |
| ATOM | 5790 | OD1 | ASP | B | 120 | 18.945 | −30.645 | −23.741 | 1.00 | 107.90 | O |
| ATOM | 5791 | OD2 | ASP | B | 120 | 18.745 | −30.915 | −25.861 | 1.00 | 113.13 | O |
| ATOM | 5792 | C | ASP | B | 120 | 20.640 | −28.408 | −22.774 | 1.00 | 75.90 | C |
| ATOM | 5793 | O | ASP | B | 120 | 21.832 | −28.332 | −23.045 | 1.00 | 78.42 | O |
| ATOM | 5794 | N | LEU | B | 121 | 20.163 | −28.841 | −21.617 | 1.00 | 75.61 | N |
| ATOM | 5796 | CA | LEU | B | 121 | 20.991 | −29.353 | −20.541 | 1.00 | 78.15 | C |
| ATOM | 5798 | CB | LEU | B | 121 | 20.789 | −28.490 | −19.290 | 1.00 | 78.05 | C |
| ATOM | 5801 | CG | LEU | B | 121 | 20.841 | −26.963 | −19.423 | 1.00 | 75.70 | C |
| ATOM | 5803 | CD1 | LEU | B | 121 | 20.695 | −26.305 | −18.055 | 1.00 | 69.76 | C |
| ATOM | 5807 | CD2 | LEU | B | 121 | 22.123 | −26.498 | −20.104 | 1.00 | 80.70 | C |
| ATOM | 5811 | C | LEU | B | 121 | 20.543 | −30.789 | −20.259 | 1.00 | 82.32 | C |
| ATOM | 5812 | O | LEU | B | 121 | 19.362 | −31.109 | −20.370 | 1.00 | 86.43 | O |
| ATOM | 5813 | N | ASP | B | 122 | 21.483 | −31.663 | −19.922 | 1.00 | 90.90 | N |
| ATOM | 5815 | CA | ASP | B | 122 | 21.138 | −33.027 | −19.532 | 1.00 | 96.46 | C |
| ATOM | 5817 | CB | ASP | B | 122 | 22.364 | −33.955 | −19.624 | 1.00 | 91.79 | C |
| ATOM | 5820 | CG | ASP | B | 122 | 23.649 | −33.306 | −19.109 | 1.00 | 100.09 | C |
| ATOM | 5821 | OD1 | ASP | B | 122 | 24.092 | −33.629 | −17.983 | 1.00 | 89.45 | O |
| ATOM | 5822 | OD2 | ASP | B | 122 | 24.319 | −32.489 | −19.780 | 1.00 | 109.42 | O |
| ATOM | 5823 | C | ASP | B | 122 | 20.535 | −33.036 | −18.118 | 1.00 | 99.89 | C |
| ATOM | 5824 | O | ASP | B | 122 | 20.946 | −32.252 | −17.265 | 1.00 | 101.21 | O |
| ATOM | 5825 | N | VAL | B | 123 | 19.554 | −33.910 | −17.885 | 1.00 | 105.89 | N |
| ATOM | 5827 | CA | VAL | B | 123 | 18.944 | −34.101 | −16.556 | 1.00 | 110.37 | C |
| ATOM | 5829 | CB | VAL | B | 123 | 17.551 | −34.783 | −16.685 | 1.00 | 109.40 | C |
| ATOM | 5831 | CG1 | VAL | B | 123 | 17.328 | −35.823 | −15.580 | 1.00 | 104.49 | C |
| ATOM | 5835 | CG2 | VAL | B | 123 | 16.451 | −33.721 | −16.691 | 1.00 | 107.39 | C |
| ATOM | 5839 | C | VAL | B | 123 | 19.834 | −34.900 | −15.578 | 1.00 | 113.92 | C |
| ATOM | 5840 | O | VAL | B | 123 | 20.091 | −36.097 | −15.784 | 1.00 | 117.21 | O |
| ATOM | 5841 | N | GLU | B | 124 | 20.234 | −34.248 | −14.483 | 1.00 | 116.41 | N |
| ATOM | 5843 | CA | GLU | B | 124 | 21.443 | −34.595 | −13.720 | 1.00 | 118.53 | C |
| ATOM | 5845 | CB | GLU | B | 124 | 21.790 | −33.431 | −12.782 | 1.00 | 119.85 | C |
| ATOM | 5848 | CG | GLU | B | 124 | 22.985 | −33.668 | −11.870 | 1.00 | 123.58 | C |
| ATOM | 5851 | CD | GLU | B | 124 | 23.643 | −32.371 | −11.430 | 1.00 | 128.31 | C |
| ATOM | 5852 | OE1 | GLU | B | 124 | 24.113 | −32.300 | −10.272 | 1.00 | 124.98 | O |
| ATOM | 5853 | OE2 | GLU | B | 124 | 23.691 | −31.420 | −12.243 | 1.00 | 131.24 | O |
| ATOM | 5854 | C | GLU | B | 124 | 21.368 | −35.889 | −12.902 | 1.00 | 117.99 | C |
| ATOM | 5855 | O | GLU | B | 124 | 21.945 | −36.910 | −13.282 | 1.00 | 114.37 | O |
| ATOM | 5856 | N | GLN | B | 125 | 20.701 | −35.806 | −11.752 | 1.00 | 119.49 | N |
| ATOM | 5858 | CA | GLN | B | 125 | 20.446 | −36.942 | −10.867 | 1.00 | 120.28 | C |
| ATOM | 5860 | CB | GLN | B | 125 | 19.624 | −38.033 | −11.578 | 1.00 | 121.75 | C |
| ATOM | 5863 | CG | GLN | B | 125 | 18.307 | −38.424 | −10.868 | 1.00 | 124.54 | C |
| ATOM | 5866 | CD | GLN | B | 125 | 17.056 | −38.308 | −11.761 | 1.00 | 131.27 | C |
| ATOM | 5867 | OE1 | GLN | B | 125 | 16.812 | −37.260 | −12.388 | 1.00 | 138.50 | O |
| ATOM | 5868 | NE2 | GLN | B | 125 | 16.239 | −39.368 | −11.779 | 1.00 | 122.66 | N |
| ATOM | 5871 | C | GLN | B | 125 | 21.721 | −37.496 | −10.217 | 1.00 | 120.10 | C |
| ATOM | 5872 | O | GLN | B | 125 | 22.831 | −37.039 | −10.499 | 1.00 | 118.35 | O |
| ATOM | 5873 | N | LEU | B | 126 | 21.533 | −38.469 | −9.329 | 1.00 | 121.84 | N |
| ATOM | 5875 | CA | LEU | B | 126 | 22.530 | −38.860 | −8.332 | 1.00 | 122.73 | C |
| ATOM | 5877 | CB | LEU | B | 126 | 23.580 | −37.760 | −8.142 | 1.00 | 123.12 | C |
| ATOM | 5880 | CG | LEU | B | 126 | 24.808 | −38.153 | −7.319 | 1.00 | 125.64 | C |
| ATOM | 5882 | CD1 | LEU | B | 126 | 25.991 | −38.438 | −8.241 | 1.00 | 123.45 | C |
| ATOM | 5886 | CD2 | LEU | B | 126 | 25.156 | −37.068 | −6.302 | 1.00 | 125.58 | C |
| ATOM | 5890 | C | LEU | B | 126 | 21.792 | −39.125 | −7.017 | 1.00 | 123.51 | C |
| ATOM | 5891 | O | LEU | B | 126 | 21.974 | −40.163 | −6.383 | 1.00 | 119.12 | O |
| ATOM | 5892 | N | GLY | B | 127 | 20.948 | −38.168 | −6.633 | 1.00 | 127.05 | N |
| ATOM | 5894 | CA | GLY | B | 127 | 19.965 | −38.334 | −5.574 | 1.00 | 128.29 | C |
| ATOM | 5897 | C | GLY | B | 127 | 20.484 | −39.060 | −4.349 | 1.00 | 130.06 | C |
| ATOM | 5898 | O | GLY | B | 127 | 19.870 | −40.036 | −3.921 | 1.00 | 131.47 | O |
| ATOM | 5899 | N | ILE | B | 128 | 21.588 | −38.579 | −3.776 | 1.00 | 130.12 | N |
| ATOM | 5901 | CA | ILE | B | 128 | 22.246 | −39.272 | −2.667 | 1.00 | 129.60 | C |
| ATOM | 5903 | CB | ILE | B | 128 | 23.199 | −38.310 | −1.910 | 1.00 | 128.51 | C |
| ATOM | 5905 | CG1 | ILE | B | 128 | 24.621 | −38.890 | −1.868 | 1.00 | 125.16 | C |
| ATOM | 5908 | CD1 | ILE | B | 128 | 25.398 | −38.746 | −3.162 | 1.00 | 118.68 | C |
| ATOM | 5912 | CG2 | ILE | B | 128 | 22.693 | −38.048 | −0.489 | 1.00 | 123.23 | C |
| ATOM | 5916 | C | ILE | B | 128 | 21.195 | −39.864 | −1.719 | 1.00 | 131.38 | C |
| ATOM | 5917 | O | ILE | B | 128 | 20.288 | −39.148 | −1.286 | 1.00 | 131.63 | O |
| ATOM | 5918 | N | PRO | B | 129 | 21.306 | −41.163 | −1.419 | 1.00 | 130.58 | N |
| ATOM | 5919 | CA | PRO | B | 129 | 20.219 | −41.930 | −0.789 | 1.00 | 128.81 | C |
| ATOM | 5921 | CB | PRO | B | 129 | 20.486 | −43.375 | −1.247 | 1.00 | 128.94 | C |
| ATOM | 5924 | CG | PRO | B | 129 | 21.836 | −43.362 | −1.966 | 1.00 | 131.15 | C |
| ATOM | 5927 | CD | PRO | B | 129 | 22.476 | −42.016 | −1.692 | 1.00 | 131.40 | C |
| ATOM | 5930 | C | PRO | B | 129 | 20.127 | −41.853 | 0.741 | 1.00 | 124.83 | C |
| ATOM | 5931 | O | PRO | B | 129 | 21.130 | −41.698 | 1.440 | 1.00 | 123.99 | O |
| ATOM | 5932 | N | GLU | B | 130 | 18.906 | −42.013 | 1.246 | 1.00 | 121.54 | N |
| ATOM | 5934 | CA | GLU | B | 130 | 18.531 | −41.564 | 2.586 | 1.00 | 118.72 | C |
| ATOM | 5936 | CB | GLU | B | 130 | 17.035 | −41.801 | 2.824 | 1.00 | 119.10 | C |
| ATOM | 5939 | CG | GLU | B | 130 | 16.133 | −40.844 | 2.045 | 1.00 | 121.73 | C |
| ATOM | 5942 | CD | GLU | B | 130 | 14.802 | −40.590 | 2.733 | 1.00 | 122.36 | C |
| ATOM | 5943 | OE1 | GLU | B | 130 | 14.791 | −39.975 | 3.826 | 1.00 | 115.76 | O |
| ATOM | 5944 | OE2 | GLU | B | 130 | 13.764 | −40.997 | 2.167 | 1.00 | 120.42 | O |
| ATOM | 5945 | C | GLU | B | 130 | 19.330 | −42.253 | 3.681 | 1.00 | 113.57 | C |
| ATOM | 5946 | O | GLU | B | 130 | 19.957 | −43.285 | 3.440 | 1.00 | 113.43 | O |
| ATOM | 5947 | N | GLN | B | 131 | 19.294 | −41.677 | 4.881 | 1.00 | 106.16 | N |
| ATOM | 5949 | CA | GLN | B | 131 | 19.967 | −42.263 | 6.037 | 1.00 | 99.82 | C |
| ATOM | 5951 | CB | GLN | B | 131 | 21.419 | −41.790 | 6.102 | 1.00 | 97.25 | C |
| ATOM | 5954 | CG | GLN | B | 131 | 21.868 | −41.046 | 4.863 | 1.00 | 94.04 | C |
| ATOM | 5957 | CD | GLN | B | 131 | 23.362 | −40.833 | 4.829 | 1.00 | 90.75 | C |
| ATOM | 5958 | OE1 | GLN | B | 131 | 24.051 | −41.059 | 5.828 | 1.00 | 77.74 | O |
| ATOM | 5959 | NE2 | GLN | B | 131 | 23.872 | −40.417 | 3.674 | 1.00 | 78.22 | N |
| ATOM | 5962 | C | GLN | B | 131 | 19.261 | −41.916 | 7.344 | 1.00 | 96.30 | C |
| ATOM | 5963 | O | GLN | B | 131 | 18.523 | −40.933 | 7.424 | 1.00 | 93.66 | O |
| ATOM | 5964 | N | GLU | B | 132 | 19.469 | −42.756 | 8.354 | 1.00 | 93.12 | N |
| ATOM | 5966 | CA | GLU | B | 132 | 19.259 | −42.354 | 9.741 | 1.00 | 88.20 | C |
| ATOM | 5968 | CB | GLU | B | 132 | 18.940 | −43.557 | 10.644 | 1.00 | 90.90 | C |
| ATOM | 5971 | CG | GLU | B | 132 | 17.963 | −43.234 | 11.779 | 1.00 | 99.88 | C |
| ATOM | 5974 | CD | GLU | B | 132 | 17.602 | −44.444 | 12.639 | 1.00 | 106.57 | C |
| ATOM | 5975 | OE1 | GLU | B | 132 | 16.809 | −45.302 | 12.175 | 1.00 | 111.38 | O |
| ATOM | 5976 | OE2 | GLU | B | 132 | 18.087 | −44.530 | 13.793 | 1.00 | 97.18 | O |
| ATOM | 5977 | C | GLU | B | 132 | 20.531 | −41.647 | 10.206 | 1.00 | 79.32 | C |
| ATOM | 5978 | O | GLU | B | 132 | 21.653 | −42.100 | 9.952 | 1.00 | 74.41 | O |
| ATOM | 5979 | N | TYR | B | 133 | 20.329 | −40.515 | 10.868 | 1.00 | 67.55 | N |
| ATOM | 5981 | CA | TYR | B | 133 | 21.409 | −39.596 | 11.182 | 1.00 | 55.35 | C |
| ATOM | 5983 | CB | TYR | B | 133 | 20.977 | −38.195 | 10.759 | 1.00 | 54.54 | C |
| ATOM | 5986 | CG | TYR | B | 133 | 21.093 | −37.921 | 9.273 | 1.00 | 35.48 | C |
| ATOM | 5987 | CD1 | TYR | B | 133 | 20.082 | −38.262 | 8.394 | 1.00 | 46.19 | C |
| ATOM | 5989 | CE1 | TYR | B | 133 | 20.198 | −38.017 | 7.014 | 1.00 | 44.59 | C |
| ATOM | 5991 | CZ | TYR | B | 133 | 21.336 | −37.431 | 6.518 | 1.00 | 39.45 | C |
| ATOM | 5992 | OH | TYR | B | 133 | 21.460 | −37.132 | 5.172 | 1.00 | 42.92 | O |
| ATOM | 5994 | CE2 | TYR | B | 133 | 22.365 | −37.095 | 7.385 | 1.00 | 51.33 | C |
| ATOM | 5996 | CD2 | TYR | B | 133 | 22.235 | −37.348 | 8.752 | 1.00 | 38.89 | C |
| ATOM | 5998 | C | TYR | B | 133 | 21.633 | −39.655 | 12.684 | 1.00 | 51.82 | C |
| ATOM | 5999 | O | TYR | B | 133 | 20.722 | −39.983 | 13.433 | 1.00 | 53.84 | O |
| ATOM | 6000 | N | SER | B | 134 | 22.829 | −39.344 | 13.156 | 1.00 | 52.01 | N |
| ATOM | 6002 | CA | SER | B | 134 | 23.042 | −39.360 | 14.596 | 1.00 | 54.34 | C |
| ATOM | 6004 | CB | SER | B | 134 | 24.466 | −38.934 | 14.934 | 1.00 | 57.09 | C |
| ATOM | 6007 | OG | SER | B | 134 | 25.410 | −39.712 | 14.224 | 1.00 | 59.72 | O |
| ATOM | 6009 | C | SER | B | 134 | 22.033 | −38.430 | 15.273 | 1.00 | 61.03 | C |
| ATOM | 6010 | O | SER | B | 134 | 21.502 | −38.740 | 16.348 | 1.00 | 61.79 | O |
| ATOM | 6011 | N | CYS | B | 135 | 21.725 | −37.315 | 14.607 | 1.00 | 62.30 | N |
| ATOM | 6013 | CA | CYS | B | 135 | 21.018 | −36.224 | 15.261 | 1.00 | 60.48 | C |
| ATOM | 6015 | CB | CYS | B | 135 | 22.019 | −35.395 | 16.061 | 1.00 | 62.35 | C |
| ATOM | 6018 | SG | CYS | B | 135 | 21.296 | −33.918 | 16.772 | 1.00 | 75.97 | S |
| ATOM | 6019 | C | CYS | B | 135 | 20.151 | −35.348 | 14.342 | 1.00 | 51.82 | C |
| ATOM | 6020 | O | CYS | B | 135 | 20.593 | −34.851 | 13.304 | 1.00 | 51.79 | O |
| ATOM | 6021 | N | VAL | B | 136 | 18.883 | −35.222 | 14.729 | 1.00 | 46.98 | N |
| ATOM | 6023 | CA | VAL | B | 136 | 17.877 | −34.509 | 13.968 | 1.00 | 39.86 | C |
| ATOM | 6025 | CB | VAL | B | 136 | 16.773 | −35.439 | 13.484 | 1.00 | 43.97 | C |
| ATOM | 6027 | CG1 | VAL | B | 136 | 15.760 | −34.616 | 12.824 | 1.00 | 31.02 | C |
| ATOM | 6031 | CG2 | VAL | B | 136 | 17.274 | −36.570 | 12.517 | 1.00 | 35.31 | C |
| ATOM | 6035 | C | VAL | B | 136 | 17.229 | −33.397 | 14.831 | 1.00 | 50.28 | C |
| ATOM | 6036 | O | VAL | B | 136 | 16.536 | −33.689 | 15.827 | 1.00 | 40.52 | O |
| ATOM | 6037 | N | VAL | B | 137 | 17.501 | −32.139 | 14.454 | 1.00 | 45.56 | N |
| ATOM | 6039 | CA | VAL | B | 137 | 17.016 | −30.955 | 15.149 | 1.00 | 43.28 | C |
| ATOM | 6041 | CB | VAL | B | 137 | 18.141 | −29.921 | 15.401 | 1.00 | 42.32 | C |
| ATOM | 6043 | CG1 | VAL | B | 137 | 17.587 | −28.649 | 16.000 | 1.00 | 39.04 | C |
| ATOM | 6047 | CG2 | VAL | B | 137 | 19.182 | −30.466 | 16.319 | 1.00 | 33.94 | C |
| ATOM | 6051 | C | VAL | B | 137 | 15.929 | −30.273 | 14.328 | 1.00 | 43.46 | C |
| ATOM | 6052 | O | VAL | B | 137 | 16.061 | −30.087 | 13.130 | 1.00 | 47.84 | O |
| ATOM | 6053 | N | LYS | B | 138 | 14.832 | −29.912 | 14.975 | 1.00 | 46.91 | N |
| ATOM | 6055 | CA | LYS | B | 138 | 13.778 | −29.179 | 14.303 | 1.00 | 47.97 | C |
| ATOM | 6057 | CB | LYS | B | 138 | 12.503 | −30.035 | 14.148 | 1.00 | 50.38 | C |
| ATOM | 6060 | CG | LYS | B | 138 | 11.324 | −29.595 | 15.004 | 1.00 | 64.53 | C |
| ATOM | 6063 | CD | LYS | B | 138 | 10.493 | −28.451 | 14.383 | 1.00 | 77.37 | C |
| ATOM | 6066 | CE | LYS | B | 138 | 9.394 | −27.931 | 15.334 | 1.00 | 64.01 | C |
| ATOM | 6069 | NZ | LYS | B | 138 | 9.496 | −26.458 | 15.555 | 1.00 | 54.34 | N |
| ATOM | 6073 | C | LYS | B | 138 | 13.546 | −27.902 | 15.096 | 1.00 | 37.53 | C |
| ATOM | 6074 | O | LYS | B | 138 | 13.523 | −27.908 | 16.317 | 1.00 | 40.32 | O |
| ATOM | 6075 | N | MET | B | 139 | 13.406 | −26.792 | 14.390 | 1.00 | 36.48 | N |
| ATOM | 6077 | CA | MET | B | 139 | 13.416 | −25.497 | 15.047 | 1.00 | 30.63 | C |
| ATOM | 6079 | CB | MET | B | 139 | 14.858 | −25.143 | 15.417 | 1.00 | 35.33 | C |
| ATOM | 6082 | CG | MET | B | 139 | 15.709 | −24.490 | 14.331 | 1.00 | 51.13 | C |
| ATOM | 6085 | SD | MET | B | 139 | 17.394 | −24.055 | 14.948 | 1.00 | 43.64 | S |
| ATOM | 6086 | CE | MET | B | 139 | 18.024 | −25.506 | 14.736 | 1.00 | 34.10 | C |
| ATOM | 6090 | C | MET | B | 139 | 12.722 | −24.446 | 14.190 | 1.00 | 26.87 | C |
| ATOM | 6091 | O | MET | B | 139 | 12.497 | −24.644 | 13.000 | 1.00 | 34.76 | O |
| ATOM | 6092 | N | PRO | B | 140 | 12.383 | −23.307 | 14.765 | 1.00 | 30.37 | N |
| ATOM | 6093 | CA | PRO | B | 140 | 11.835 | −22.222 | 13.950 | 1.00 | 29.88 | C |
| ATOM | 6095 | CB | PRO | B | 140 | 11.585 | −21.100 | 14.958 | 1.00 | 32.34 | C |
| ATOM | 6098 | CG | PRO | B | 140 | 11.610 | −21.763 | 16.287 | 1.00 | 35.58 | C |
| ATOM | 6101 | CD | PRO | B | 140 | 12.619 | −22.902 | 16.163 | 1.00 | 35.53 | C |
| ATOM | 6104 | C | PRO | B | 140 | 12.851 | −21.759 | 12.900 | 1.00 | 32.24 | C |
| ATOM | 6105 | O | PRO | B | 140 | 13.997 | −21.483 | 13.251 | 1.00 | 32.25 | O |
| ATOM | 6106 | N | SER | B | 141 | 12.377 | −21.617 | 11.666 | 1.00 | 26.11 | N |
| ATOM | 6108 | CA | SER | B | 141 | 13.168 | −21.241 | 10.537 | 1.00 | 36.19 | C |
| ATOM | 6110 | CB | SER | B | 141 | 12.330 | −21.410 | 9.269 | 1.00 | 23.54 | C |
| ATOM | 6113 | OG | SER | B | 141 | 11.247 | −20.538 | 9.248 | 1.00 | 36.11 | O |
| ATOM | 6115 | C | SER | B | 141 | 13.717 | −19.816 | 10.711 | 1.00 | 36.92 | C |
| ATOM | 6116 | O | SER | B | 141 | 14.888 | −19.581 | 10.459 | 1.00 | 33.06 | O |
| ATOM | 6117 | N | GLY | B | 142 | 12.914 | −18.883 | 11.204 | 1.00 | 28.79 | N |
| ATOM | 6119 | CA | GLY | B | 142 | 13.374 | −17.501 | 11.336 | 1.00 | 18.93 | C |
| ATOM | 6122 | C | GLY | B | 142 | 14.425 | −17.399 | 12.444 | 1.00 | 26.91 | C |
| ATOM | 6123 | O | GLY | B | 142 | 15.310 | −16.553 | 12.428 | 1.00 | 31.69 | O |
| ATOM | 6124 | N | GLU | B | 143 | 14.389 | −18.320 | 13.395 | 1.00 | 25.29 | N |
| ATOM | 6126 | CA | GLU | B | 143 | 15.416 | −18.349 | 14.425 | 1.00 | 30.82 | C |
| ATOM | 6128 | CB | GLU | B | 143 | 14.975 | −19.196 | 15.593 | 1.00 | 25.97 | C |
| ATOM | 6131 | CG | GLU | B | 143 | 15.890 | −19.066 | 16.795 | 1.00 | 33.82 | C |
| ATOM | 6134 | CD | GLU | B | 143 | 16.023 | −17.606 | 17.198 | 1.00 | 42.66 | C |
| ATOM | 6135 | OE1 | GLU | B | 143 | 14.978 | −16.929 | 17.294 | 1.00 | 51.45 | O |
| ATOM | 6136 | OE2 | GLU | B | 143 | 17.159 | −17.129 | 17.403 | 1.00 | 56.24 | O |
| ATOM | 6137 | C | GLU | B | 143 | 16.719 | −18.958 | 13.891 | 1.00 | 33.65 | C |
| ATOM | 6138 | O | GLU | B | 143 | 17.814 | −18.589 | 14.307 | 1.00 | 31.13 | O |
| ATOM | 6139 | N | PHE | B | 144 | 16.594 | −19.902 | 12.974 | 1.00 | 29.66 | N |
| ATOM | 6141 | CA | PHE | B | 144 | 17.799 | −20.491 | 12.409 | 1.00 | 33.75 | C |
| ATOM | 6143 | CB | PHE | B | 144 | 17.502 | −21.808 | 11.671 | 1.00 | 22.51 | C |
| ATOM | 6146 | CG | PHE | B | 144 | 18.729 | −22.417 | 11.031 | 1.00 | 38.96 | C |
| ATOM | 6147 | CD1 | PHE | B | 144 | 19.758 | −22.922 | 11.805 | 1.00 | 29.81 | C |
| ATOM | 6149 | CE1 | PHE | B | 144 | 20.890 | −23.465 | 11.224 | 1.00 | 33.75 | C |
| ATOM | 6151 | CZ | PHE | B | 144 | 20.984 | −23.530 | 9.860 | 1.00 | 38.58 | C |
| ATOM | 6153 | CE2 | PHE | B | 144 | 19.929 | −23.071 | 9.086 | 1.00 | 23.32 | C |
| ATOM | 6155 | CD2 | PHE | B | 144 | 18.848 | −22.482 | 9.659 | 1.00 | 29.39 | C |
| ATOM | 6157 | C | PHE | B | 144 | 18.461 | −19.468 | 11.517 | 1.00 | 24.25 | C |
| ATOM | 6158 | O | PHE | B | 144 | 19.681 | −19.311 | 11.525 | 1.00 | 32.34 | O |
| ATOM | 6159 | N | ALA | B | 145 | 17.647 | −18.777 | 10.723 | 1.00 | 23.29 | N |
| ATOM | 6161 | CA | ALA | B | 145 | 18.146 | −17.703 | 9.888 | 1.00 | 25.99 | C |
| ATOM | 6163 | CB | ALA | B | 145 | 17.029 | −17.062 | 9.132 | 1.00 | 25.87 | C |
| ATOM | 6167 | C | ALA | B | 145 | 18.912 | −16.637 | 10.684 | 1.00 | 30.19 | C |
| ATOM | 6168 | O | ALA | B | 145 | 19.882 | −16.065 | 10.203 | 1.00 | 32.78 | O |
| ATOM | 6169 | N | ARG | B | 146 | 18.433 | −16.324 | 11.876 | 1.00 | 30.85 | N |
| ATOM | 6171 | CA | ARG | B | 146 | 18.957 | −15.189 | 12.614 | 1.00 | 33.14 | C |
| ATOM | 6173 | CB | ARG | B | 146 | 17.919 | −14.699 | 13.662 | 1.00 | 31.82 | C |
| ATOM | 6176 | CG | ARG | B | 146 | 18.395 | −14.784 | 15.101 | 1.00 | 35.30 | C |
| ATOM | 6179 | CD | ARG | B | 146 | 17.681 | −13.865 | 16.103 | 1.00 | 74.96 | C |
| ATOM | 6182 | NE | ARG | B | 146 | 18.634 | −12.996 | 16.806 | 1.00 | 73.18 | N |
| ATOM | 6184 | CZ | ARG | B | 146 | 18.313 | −11.914 | 17.506 | 1.00 | 66.63 | C |
| ATOM | 6185 | NH1 | ARG | B | 146 | 17.041 | −11.536 | 17.666 | 1.00 | 61.84 | N |
| ATOM | 6188 | NH2 | ARG | B | 146 | 19.287 | −11.232 | 18.081 | 1.00 | 62.97 | N |
| ATOM | 6191 | C | ARG | B | 146 | 20.257 | −15.588 | 13.278 | 1.00 | 23.39 | C |
| ATOM | 6192 | O | ARG | B | 146 | 21.140 | −14.755 | 13.453 | 1.00 | 30.96 | O |
| ATOM | 6193 | N | ILE | B | 147 | 20.341 | −16.849 | 13.678 | 1.00 | 24.15 | N |
| ATOM | 6195 | CA | ILE | B | 147 | 21.538 | −17.396 | 14.298 | 1.00 | 27.17 | C |
| ATOM | 6197 | CB | ILE | B | 147 | 21.252 | −18.801 | 14.836 | 1.00 | 32.42 | C |
| ATOM | 6199 | CG1 | ILE | B | 147 | 20.376 | −18.714 | 16.088 | 1.00 | 37.29 | C |
| ATOM | 6202 | CD1 | ILE | B | 147 | 19.589 | −19.984 | 16.405 | 1.00 | 32.02 | C |
| ATOM | 6206 | CG2 | ILE | B | 147 | 22.540 | −19.544 | 15.106 | 1.00 | 25.76 | C |
| ATOM | 6210 | C | ILE | B | 147 | 22.684 | −17.447 | 13.277 | 1.00 | 29.61 | C |
| ATOM | 6211 | O | ILE | B | 147 | 23.770 | −16.990 | 13.575 | 1.00 | 28.43 | O |
| ATOM | 6212 | N | CYS | B | 148 | 22.418 | −17.821 | 12.037 | 1.00 | 23.58 | N |
| ATOM | 6214 | CA | CYS | B | 148 | 23.493 | −17.884 | 11.048 | 1.00 | 30.10 | C |
| ATOM | 6216 | CB | CYS | B | 148 | 23.025 | −18.584 | 9.772 | 1.00 | 33.77 | C |
| ATOM | 6219 | SG | CYS | B | 148 | 22.740 | −20.327 | 10.046 | 1.00 | 30.86 | S |
| ATOM | 6220 | C | CYS | B | 148 | 23.959 | −16.495 | 10.704 | 1.00 | 31.22 | C |
| ATOM | 6221 | O | CYS | B | 148 | 25.151 | −16.254 | 10.533 | 1.00 | 30.23 | O |
| ATOM | 6222 | N | ARG | B | 149 | 23.009 | −15.583 | 10.550 | 1.00 | 26.13 | N |
| ATOM | 6224 | CA | ARG | B | 149 | 23.361 | −14.211 | 10.209 | 1.00 | 31.96 | C |
| ATOM | 6226 | CB | ARG | B | 149 | 22.110 | −13.383 | 9.904 | 1.00 | 21.47 | C |
| ATOM | 6229 | CG | ARG | B | 149 | 22.392 | −11.872 | 9.897 | 1.00 | 42.63 | C |
| ATOM | 6232 | CD | ARG | B | 149 | 21.308 | −10.994 | 9.326 | 1.00 | 57.77 | C |
| ATOM | 6235 | NE | ARG | B | 149 | 20.030 | −11.699 | 9.225 | 1.00 | 86.45 | N |
| ATOM | 6237 | CZ | ARG | B | 149 | 19.099 | −11.764 | 10.180 | 1.00 | 91.98 | C |
| ATOM | 6238 | NH1 | ARG | B | 149 | 19.275 | −11.177 | 11.367 | 1.00 | 72.08 | N |
| ATOM | 6241 | NH2 | ARG | B | 149 | 17.978 | −12.441 | 9.936 | 1.00 | 84.88 | N |
| ATOM | 6244 | C | ARG | B | 149 | 24.222 | −13.526 | 11.281 | 1.00 | 33.53 | C |
| ATOM | 6245 | O | ARG | B | 149 | 25.229 | −12.907 | 10.971 | 1.00 | 37.65 | O |
| ATOM | 6246 | N | ASP | B | 150 | 23.799 | −13.605 | 12.535 | 1.00 | 31.38 | N |
| ATOM | 6248 | CA | ASP | B | 150 | 24.545 | −13.025 | 13.641 | 1.00 | 31.63 | C |
| ATOM | 6250 | CB | ASP | B | 150 | 23.773 | −13.209 | 14.960 | 1.00 | 26.47 | C |
| ATOM | 6253 | CG | ASP | B | 150 | 22.479 | −12.411 | 14.994 | 1.00 | 29.46 | C |
| ATOM | 6254 | OD1 | ASP | B | 150 | 22.243 | −11.580 | 14.095 | 1.00 | 31.52 | O |
| ATOM | 6255 | OD2 | ASP | B | 150 | 21.621 | −12.599 | 15.859 | 1.00 | 43.19 | O |
| ATOM | 6256 | C | ASP | B | 150 | 25.951 | −13.603 | 13.817 | 1.00 | 29.07 | C |
| ATOM | 6257 | O | ASP | B | 150 | 26.883 | −12.871 | 14.124 | 1.00 | 26.07 | O |
| ATOM | 6258 | N | LEU | B | 151 | 26.079 | −14.921 | 13.785 | 1.00 | 25.43 | N |
| ATOM | 6260 | CA | LEU | B | 151 | 27.367 | −15.510 | 14.056 | 1.00 | 26.55 | C |
| ATOM | 6262 | CB | LEU | B | 151 | 27.240 | −17.008 | 14.209 | 1.00 | 23.24 | C |
| ATOM | 6265 | CG | LEU | B | 151 | 26.526 | −17.390 | 15.503 | 1.00 | 27.12 | C |
| ATOM | 6267 | CD1 | LEU | B | 151 | 26.336 | −18.856 | 15.555 | 1.00 | 27.28 | C |
| ATOM | 6271 | CD2 | LEU | B | 151 | 27.310 | −16.944 | 16.714 | 1.00 | 32.31 | C |
| ATOM | 6275 | C | LEU | B | 151 | 28.351 | −15.171 | 12.964 | 1.00 | 27.78 | C |
| ATOM | 6276 | O | LEU | B | 151 | 29.550 | −15.325 | 13.154 | 1.00 | 34.74 | O |
| ATOM | 6277 | N | SER | B | 152 | 27.805 | −14.831 | 11.800 | 1.00 | 33.22 | N |
| ATOM | 6279 | CA | SER | B | 152 | 28.582 | −14.491 | 10.638 | 1.00 | 32.24 | C |
| ATOM | 6281 | CB | SER | B | 152 | 27.739 | −14.556 | 9.354 | 1.00 | 28.94 | C |
| ATOM | 6284 | OG | SER | B | 152 | 26.999 | −13.376 | 9.184 | 1.00 | 40.03 | O |
| ATOM | 6286 | C | SER | B | 152 | 29.264 | −13.146 | 10.836 | 1.00 | 33.19 | C |
| ATOM | 6287 | O | SER | B | 152 | 30.268 | −12.886 | 10.182 | 1.00 | 41.58 | O |
| ATOM | 6288 | N | HIS | B | 153 | 28.804 | −12.342 | 11.797 | 1.00 | 41.57 | N |
| ATOM | 6290 | CA | HIS | B | 153 | 29.575 | −11.160 | 12.244 | 1.00 | 39.97 | C |
| ATOM | 6292 | CB | HIS | B | 153 | 28.707 | −10.158 | 13.020 | 1.00 | 43.34 | C |
| ATOM | 6295 | CG | HIS | B | 153 | 27.637 | −9.497 | 12.205 | 1.00 | 46.95 | C |
| ATOM | 6296 | ND1 | HIS | B | 153 | 27.868 | −8.937 | 10.967 | 1.00 | 74.50 | N |
| ATOM | 6298 | CE1 | HIS | B | 153 | 26.735 | −8.457 | 10.481 | 1.00 | 66.07 | C |
| ATOM | 6300 | NE2 | HIS | B | 153 | 25.773 | −8.705 | 11.352 | 1.00 | 55.03 | N |
| ATOM | 6302 | CD2 | HIS | B | 153 | 26.318 | −9.314 | 12.454 | 1.00 | 51.98 | C |
| ATOM | 6304 | C | HIS | B | 153 | 30.811 | −11.490 | 13.102 | 1.00 | 44.17 | C |
| ATOM | 6305 | O | HIS | B | 153 | 31.693 | −10.641 | 13.250 | 1.00 | 58.04 | O |
| ATOM | 6306 | N | ILE | B | 154 | 30.854 | −12.672 | 13.714 | 1.00 | 38.72 | N |
| ATOM | 6308 | CA | ILE | B | 154 | 31.913 | −13.059 | 14.644 | 1.00 | 41.51 | C |
| ATOM | 6310 | CB | ILE | B | 154 | 31.330 | −13.990 | 15.725 | 1.00 | 36.52 | C |
| ATOM | 6312 | CG1 | ILE | B | 154 | 30.114 | −13.374 | 16.423 | 1.00 | 42.68 | C |
| ATOM | 6315 | CD1 | ILE | B | 154 | 30.341 | −11.942 | 16.940 | 1.00 | 38.34 | C |
| ATOM | 6319 | CG2 | ILE | B | 154 | 32.405 | −14.293 | 16.733 | 1.00 | 38.91 | C |
| ATOM | 6323 | C | ILE | B | 154 | 32.995 | −13.905 | 13.931 | 1.00 | 50.55 | C |
| ATOM | 6324 | O | ILE | B | 154 | 34.209 | −13.753 | 14.155 | 1.00 | 48.22 | O |
| ATOM | 6325 | N | GLY | B | 155 | 32.501 | −14.850 | 13.136 | 1.00 | 43.62 | N |
| ATOM | 6327 | CA | GLY | B | 155 | 33.298 | −15.883 | 12.508 | 1.00 | 47.45 | C |
| ATOM | 6330 | C | GLY | B | 155 | 32.830 | −16.240 | 11.099 | 1.00 | 43.70 | C |
| ATOM | 6331 | O | GLY | B | 155 | 31.820 | −15.733 | 10.611 | 1.00 | 52.49 | O |
| ATOM | 6332 | N | ASP | B | 156 | 33.571 | −17.134 | 10.450 | 1.00 | 39.30 | N |
| ATOM | 6334 | CA | ASP | B | 156 | 33.224 | −17.636 | 9.131 | 1.00 | 39.16 | C |
| ATOM | 6336 | CB | ASP | B | 156 | 34.437 | −17.552 | 8.197 | 1.00 | 43.76 | C |
| ATOM | 6339 | CG | ASP | B | 156 | 35.744 | −17.978 | 8.871 | 1.00 | 78.31 | C |
| ATOM | 6340 | OD1 | ASP | B | 156 | 36.689 | −17.149 | 8.862 | 1.00 | 105.63 | O |
| ATOM | 6341 | OD2 | ASP | B | 156 | 35.934 | −19.104 | 9.408 | 1.00 | 99.01 | O |
| ATOM | 6342 | C | ASP | B | 156 | 32.706 | −19.071 | 9.197 | 1.00 | 37.19 | C |
| ATOM | 6343 | O | ASP | B | 156 | 32.250 | −19.620 | 8.203 | 1.00 | 40.05 | O |
| ATOM | 6344 | N | ALA | B | 157 | 32.770 | −19.668 | 10.376 | 1.00 | 36.68 | N |
| ATOM | 6346 | CA | ALA | B | 157 | 32.400 | −21.052 | 10.564 | 1.00 | 37.79 | C |
| ATOM | 6348 | CB | ALA | B | 157 | 33.642 | −21.872 | 10.659 | 1.00 | 35.70 | C |
| ATOM | 6352 | C | ALA | B | 157 | 31.609 | −21.132 | 11.873 | 1.00 | 35.33 | C |
| ATOM | 6353 | O | ALA | B | 157 | 31.896 | −20.391 | 12.827 | 1.00 | 40.88 | O |
| ATOM | 6354 | N | VAL | B | 158 | 30.621 | −22.026 | 11.885 | 1.00 | 34.96 | N |
| ATOM | 6356 | CA | VAL | B | 158 | 29.837 | −22.322 | 13.055 | 1.00 | 25.70 | C |
| ATOM | 6358 | CB | VAL | B | 158 | 28.326 | −21.971 | 12.817 | 1.00 | 34.72 | C |
| ATOM | 6360 | CG1 | VAL | B | 158 | 27.699 | −22.858 | 11.757 | 1.00 | 34.01 | C |
| ATOM | 6364 | CG2 | VAL | B | 158 | 27.564 | −22.142 | 14.088 | 1.00 | 20.11 | C |
| ATOM | 6368 | C | VAL | B | 158 | 29.957 | −23.768 | 13.464 | 1.00 | 31.37 | C |
| ATOM | 6369 | O | VAL | B | 158 | 29.989 | −24.666 | 12.633 | 1.00 | 37.33 | O |
| ATOM | 6370 | N | VAL | B | 159 | 29.914 | −24.011 | 14.772 | 1.00 | 39.82 | N |
| ATOM | 6372 | CA | VAL | B | 159 | 30.071 | −25.351 | 15.290 | 1.00 | 34.13 | C |
| ATOM | 6374 | CB | VAL | B | 159 | 31.208 | −25.393 | 16.352 | 1.00 | 39.50 | C |
| ATOM | 6376 | CG1 | VAL | B | 159 | 31.233 | −26.756 | 17.061 | 1.00 | 36.91 | C |
| ATOM | 6380 | CG2 | VAL | B | 159 | 32.588 | −25.047 | 15.746 | 1.00 | 30.87 | C |
| ATOM | 6384 | C | VAL | B | 159 | 28.727 | −25.672 | 15.943 | 1.00 | 40.08 | C |
| ATOM | 6385 | O | VAL | B | 159 | 28.324 | −25.071 | 16.939 | 1.00 | 47.69 | O |
| ATOM | 6386 | N | ILE | B | 160 | 28.007 | −26.602 | 15.349 | 1.00 | 44.34 | N |
| ATOM | 6388 | CA | ILE | B | 160 | 26.746 | −26.993 | 15.887 | 1.00 | 44.89 | C |
| ATOM | 6390 | CB | ILE | B | 160 | 25.831 | −27.367 | 14.740 | 1.00 | 44.56 | C |
| ATOM | 6392 | CG1 | ILE | B | 160 | 25.783 | −26.204 | 13.750 | 1.00 | 35.46 | C |
| ATOM | 6395 | CD1 | ILE | B | 160 | 24.738 | −26.403 | 12.630 | 1.00 | 43.52 | C |
| ATOM | 6399 | CG2 | ILE | B | 160 | 24.418 | −27.736 | 15.263 | 1.00 | 39.58 | C |
| ATOM | 6403 | C | ILE | B | 160 | 27.018 | −28.160 | 16.832 | 1.00 | 53.31 | C |
| ATOM | 6404 | O | ILE | B | 160 | 27.582 | −29.175 | 16.413 | 1.00 | 55.80 | O |
| ATOM | 6405 | N | SER | B | 161 | 26.673 | −27.971 | 18.106 | 1.00 | 47.78 | N |
| ATOM | 6407 | CA | SER | B | 161 | 26.766 | −29.012 | 19.127 | 1.00 | 54.34 | C |
| ATOM | 6409 | CB | SER | B | 161 | 27.507 | −28.541 | 20.380 | 1.00 | 49.28 | C |
| ATOM | 6412 | OG | SER | B | 161 | 28.726 | −27.901 | 20.060 | 1.00 | 73.34 | O |
| ATOM | 6414 | C | SER | B | 161 | 25.369 | −29.410 | 19.552 | 1.00 | 54.17 | C |
| ATOM | 6415 | O | SER | B | 161 | 24.614 | −28.638 | 20.136 | 1.00 | 59.77 | O |
| ATOM | 6416 | N | CYS | B | 162 | 25.030 | −30.648 | 19.267 | 1.00 | 55.84 | N |
| ATOM | 6418 | CA | CYS | B | 162 | 23.762 | −31.163 | 19.711 | 1.00 | 58.76 | C |
| ATOM | 6420 | CB | CYS | B | 162 | 23.123 | −31.943 | 18.581 | 1.00 | 52.97 | C |
| ATOM | 6423 | SG | CYS | B | 162 | 21.367 | −32.113 | 18.881 | 1.00 | 80.89 | S |
| ATOM | 6424 | C | CYS | B | 162 | 23.896 | −32.037 | 20.945 | 1.00 | 58.91 | C |
| ATOM | 6425 | O | CYS | B | 162 | 24.832 | −32.819 | 21.099 | 1.00 | 68.18 | O |
| ATOM | 6426 | N | ALA | B | 163 | 22.913 | −31.930 | 21.820 | 1.00 | 64.70 | N |
| ATOM | 6428 | CA | ALA | B | 163 | 22.948 | −32.646 | 23.078 | 1.00 | 64.43 | C |
| ATOM | 6430 | CB | ALA | B | 163 | 23.782 | −31.877 | 24.089 | 1.00 | 64.59 | C |
| ATOM | 6434 | C | ALA | B | 163 | 21.507 | −32.766 | 23.539 | 1.00 | 63.75 | C |
| ATOM | 6435 | O | ALA | B | 163 | 20.618 | −32.200 | 22.899 | 1.00 | 60.61 | O |
| ATOM | 6436 | N | LYS | B | 164 | 21.294 | −33.501 | 24.629 | 1.00 | 63.85 | N |
| ATOM | 6438 | CA | LYS | B | 164 | 19.969 | −33.670 | 25.226 | 1.00 | 70.67 | C |
| ATOM | 6440 | CB | LYS | B | 164 | 20.011 | −34.730 | 26.341 | 1.00 | 75.04 | C |
| ATOM | 6443 | CG | LYS | B | 164 | 19.012 | −35.904 | 26.206 | 1.00 | 76.17 | C |
| ATOM | 6446 | CD | LYS | B | 164 | 18.265 | −36.177 | 27.530 | 1.00 | 93.88 | C |
| ATOM | 6449 | CE | LYS | B | 164 | 19.031 | −37.129 | 28.471 | 1.00 | 96.24 | C |
| ATOM | 6452 | NZ | LYS | B | 164 | 18.441 | −38.507 | 28.539 | 1.00 | 78.32 | N |
| ATOM | 6456 | C | LYS | B | 164 | 19.482 | −32.344 | 25.814 | 1.00 | 75.89 | C |
| ATOM | 6457 | O | LYS | B | 164 | 18.272 | −32.079 | 25.812 | 1.00 | 74.59 | O |
| ATOM | 6458 | N | ASP | B | 165 | 20.420 | −31.525 | 26.309 | 1.00 | 69.18 | N |
| ATOM | 6460 | CA | ASP | B | 165 | 20.106 | −30.167 | 26.760 | 1.00 | 72.33 | C |
| ATOM | 6462 | CB | ASP | B | 165 | 21.394 | −29.361 | 27.012 | 1.00 | 74.13 | C |
| ATOM | 6465 | CG | ASP | B | 165 | 22.123 | −29.766 | 28.290 | 1.00 | 88.80 | C |
| ATOM | 6466 | OD1 | ASP | B | 165 | 22.628 | −30.910 | 28.373 | 1.00 | 103.32 | O |
| ATOM | 6467 | OD2 | ASP | B | 165 | 22.307 | −28.982 | 29.248 | 1.00 | 98.56 | O |
| ATOM | 6468 | C | ASP | B | 165 | 19.279 | −29.452 | 25.675 | 1.00 | 70.40 | C |
| ATOM | 6469 | O | ASP | B | 165 | 18.206 | −28.877 | 25.937 | 1.00 | 55.62 | O |
| ATOM | 6470 | N | GLY | B | 166 | 19.787 | −29.545 | 24.446 | 1.00 | 63.18 | N |
| ATOM | 6472 | CA | GLY | B | 166 | 19.390 | −28.671 | 23.357 | 1.00 | 56.95 | C |
| ATOM | 6475 | C | GLY | B | 166 | 20.586 | −28.464 | 22.448 | 1.00 | 56.05 | C |
| ATOM | 6476 | O | GLY | B | 166 | 21.558 | −29.230 | 22.485 | 1.00 | 54.51 | O |
| ATOM | 6477 | N | VAL | B | 167 | 20.540 | −27.418 | 21.632 | 1.00 | 51.61 | N |
| ATOM | 6479 | CA | VAL | B | 167 | 21.531 | −27.276 | 20.574 | 1.00 | 44.60 | C |
| ATOM | 6481 | CB | VAL | B | 167 | 20.878 | −27.479 | 19.185 | 1.00 | 44.50 | C |
| ATOM | 6483 | CG1 | VAL | B | 167 | 19.612 | −26.635 | 19.051 | 1.00 | 56.03 | C |
| ATOM | 6487 | CG2 | VAL | B | 167 | 21.863 | −27.216 | 18.057 | 1.00 | 42.67 | C |
| ATOM | 6491 | C | VAL | B | 167 | 22.270 | −25.952 | 20.766 | 1.00 | 48.50 | C |
| ATOM | 6492 | O | VAL | B | 167 | 21.738 | −24.979 | 21.318 | 1.00 | 53.73 | O |
| ATOM | 6493 | N | LYS | B | 168 | 23.535 | −25.960 | 20.385 | 1.00 | 43.04 | N |
| ATOM | 6495 | CA | LYS | B | 168 | 24.434 | −24.860 | 20.627 | 1.00 | 47.80 | C |
| ATOM | 6497 | CB | LYS | B | 168 | 25.446 | −25.273 | 21.708 | 1.00 | 45.92 | C |
| ATOM | 6500 | CG | LYS | B | 168 | 26.461 | −24.202 | 22.097 | 1.00 | 62.26 | C |
| ATOM | 6503 | CD | LYS | B | 168 | 27.152 | −24.529 | 23.429 | 1.00 | 67.16 | C |
| ATOM | 6506 | CE | LYS | B | 168 | 28.668 | −24.434 | 23.306 | 1.00 | 72.02 | C |
| ATOM | 6509 | NZ | LYS | B | 168 | 29.367 | −24.690 | 24.599 | 1.00 | 64.05 | N |
| ATOM | 6513 | C | LYS | B | 168 | 25.140 | −24.537 | 19.313 | 1.00 | 43.30 | C |
| ATOM | 6514 | O | LYS | B | 168 | 25.666 | −25.445 | 18.651 | 1.00 | 40.08 | O |
| ATOM | 6515 | N | PHE | B | 169 | 25.130 | −23.258 | 18.923 | 1.00 | 36.12 | N |
| ATOM | 6517 | CA | PHE | B | 169 | 25.938 | −22.794 | 17.788 | 1.00 | 31.72 | C |
| ATOM | 6519 | CB | PHE | B | 169 | 25.055 | −22.057 | 16.773 | 1.00 | 31.60 | C |
| ATOM | 6522 | CG | PHE | B | 169 | 23.722 | −22.709 | 16.558 | 1.00 | 24.86 | C |
| ATOM | 6523 | CD1 | PHE | B | 169 | 22.707 | −22.564 | 17.501 | 1.00 | 34.04 | C |
| ATOM | 6525 | CE1 | PHE | B | 169 | 21.501 | −23.229 | 17.357 | 1.00 | 37.24 | C |
| ATOM | 6527 | CZ | PHE | B | 169 | 21.278 | −24.009 | 16.262 | 1.00 | 34.25 | C |
| ATOM | 6529 | CE2 | PHE | B | 169 | 22.272 | −24.152 | 15.280 | 1.00 | 41.08 | C |
| ATOM | 6531 | CD2 | PHE | B | 169 | 23.499 | −23.514 | 15.448 | 1.00 | 31.00 | C |
| ATOM | 6533 | C | PHE | B | 169 | 27.001 | −21.850 | 18.305 | 1.00 | 34.20 | C |
| ATOM | 6534 | O | PHE | B | 169 | 26.686 | −20.932 | 19.051 | 1.00 | 50.00 | O |
| ATOM | 6535 | N | SER | B | 170 | 28.241 | −22.033 | 17.870 | 1.00 | 41.14 | N |
| ATOM | 6537 | CA | SER | B | 170 | 29.365 | −21.284 | 18.409 | 1.00 | 39.62 | C |
| ATOM | 6539 | CB | SER | B | 170 | 30.100 | −22.151 | 19.442 | 1.00 | 44.48 | C |
| ATOM | 6542 | OG | SER | B | 170 | 31.487 | −21.840 | 19.464 | 1.00 | 65.64 | O |
| ATOM | 6544 | C | SER | B | 170 | 30.285 | −20.930 | 17.243 | 1.00 | 37.69 | C |
| ATOM | 6545 | O | SER | B | 170 | 30.467 | −21.721 | 16.338 | 1.00 | 50.12 | O |
| ATOM | 6546 | N | ALA | B | 171 | 30.812 | −19.715 | 17.221 | 1.00 | 37.68 | N |
| ATOM | 6548 | CA | ALA | B | 171 | 31.648 | −19.254 | 16.130 | 1.00 | 38.54 | C |
| ATOM | 6550 | CB | ALA | B | 171 | 30.829 | −18.351 | 15.111 | 1.00 | 29.06 | C |
| ATOM | 6554 | C | ALA | B | 171 | 32.773 | −18.458 | 16.787 | 1.00 | 39.02 | C |
| ATOM | 6555 | O | ALA | B | 171 | 32.606 | −17.941 | 17.886 | 1.00 | 47.17 | O |
| ATOM | 6556 | N | SER | B | 172 | 33.864 | −18.275 | 16.056 | 1.00 | 40.93 | N |
| ATOM | 6558 | CA | SER | B | 172 | 35.105 | −17.718 | 16.584 | 1.00 | 41.00 | C |
| ATOM | 6560 | CB | SER | B | 172 | 35.935 | −18.835 | 17.206 | 1.00 | 45.38 | C |
| ATOM | 6563 | OG | SER | B | 172 | 36.799 | −18.296 | 18.181 | 1.00 | 60.95 | O |
| ATOM | 6565 | C | SER | B | 172 | 35.880 | −17.096 | 15.445 | 1.00 | 39.45 | C |
| ATOM | 6566 | O | SER | B | 172 | 35.970 | −17.671 | 14.370 | 1.00 | 49.19 | O |
| ATOM | 6567 | N | GLY | B | 173 | 36.376 | −15.888 | 15.658 | 1.00 | 40.12 | N |
| ATOM | 6569 | CA | GLY | B | 173 | 37.154 | −15.180 | 14.660 | 1.00 | 40.34 | C |
| ATOM | 6572 | C | GLY | B | 173 | 38.028 | −14.129 | 15.321 | 1.00 | 40.67 | C |
| ATOM | 6573 | O | GLY | B | 173 | 38.429 | −14.266 | 16.473 | 1.00 | 53.03 | O |
| ATOM | 6574 | N | GLU | B | 174 | 38.284 | −13.049 | 14.603 | 1.00 | 50.60 | N |
| ATOM | 6576 | CA | GLU | B | 174 | 39.216 | −12.020 | 15.059 | 1.00 | 58.01 | C |
| ATOM | 6578 | CB | GLU | B | 174 | 39.469 | −10.967 | 13.965 | 1.00 | 50.21 | C |
| ATOM | 6581 | CG | GLU | B | 174 | 39.782 | −11.525 | 12.586 | 1.00 | 70.90 | C |
| ATOM | 6584 | CD | GLU | B | 174 | 38.717 | −12.480 | 12.053 | 1.00 | 88.98 | C |
| ATOM | 6585 | OE1 | GLU | B | 174 | 37.675 | −11.973 | 11.562 | 1.00 | 92.00 | O |
| ATOM | 6586 | OE2 | GLU | B | 174 | 38.937 | −13.728 | 12.108 | 1.00 | 68.19 | O |
| ATOM | 6587 | C | GLU | B | 174 | 38.662 | −11.295 | 16.285 | 1.00 | 55.83 | C |
| ATOM | 6588 | O | GLU | B | 174 | 39.418 | −10.873 | 17.163 | 1.00 | 55.28 | O |
| ATOM | 6589 | N | LEU | B | 175 | 37.360 | −11.034 | 16.303 | 1.00 | 48.29 | N |
| ATOM | 6591 | CA | LEU | B | 175 | 36.901 | −10.057 | 17.277 | 1.00 | 46.40 | C |
| ATOM | 6593 | CB | LEU | B | 175 | 35.700 | −9.236 | 16.804 | 1.00 | 45.43 | C |
| ATOM | 6596 | CG | LEU | B | 175 | 34.889 | −9.598 | 15.564 | 1.00 | 63.58 | C |
| ATOM | 6598 | CD1 | LEU | B | 175 | 33.412 | −9.337 | 15.871 | 1.00 | 70.89 | C |
| ATOM | 6602 | CD2 | LEU | B | 175 | 35.378 | −8.776 | 14.343 | 1.00 | 64.85 | C |
| ATOM | 6606 | C | LEU | B | 175 | 36.647 | −10.752 | 18.595 | 1.00 | 28.77 | C |
| ATOM | 6607 | O | LEU | B | 175 | 36.642 | −10.145 | 19.634 | 1.00 | 43.58 | O |
| ATOM | 6608 | N | GLY | B | 176 | 36.471 | −12.055 | 18.530 | 1.00 | 31.75 | N |
| ATOM | 6610 | CA | GLY | B | 176 | 36.304 | −12.837 | 19.725 | 1.00 | 33.94 | C |
| ATOM | 6613 | C | GLY | B | 176 | 35.430 | −14.027 | 19.372 | 1.00 | 43.75 | C |
| ATOM | 6614 | O | GLY | B | 176 | 35.524 | −14.547 | 18.255 | 1.00 | 41.22 | O |
| ATOM | 6615 | N | ASN | B | 177 | 34.529 | −14.396 | 20.285 | 1.00 | 39.63 | N |
| ATOM | 6617 | CA | ASN | B | 177 | 33.733 | −15.601 | 20.136 | 1.00 | 39.40 | C |
| ATOM | 6619 | CB | ASN | B | 177 | 34.448 | −16.823 | 20.721 | 1.00 | 40.21 | C |
| ATOM | 6622 | CG | ASN | B | 177 | 34.676 | −16.729 | 22.222 | 1.00 | 53.58 | C |
| ATOM | 6623 | OD1 | ASN | B | 177 | 33.767 | −16.968 | 23.033 | 1.00 | 45.89 | O |
| ATOM | 6624 | ND2 | ASN | B | 177 | 35.932 | −16.459 | 22.599 | 1.00 | 50.98 | N |
| ATOM | 6627 | C | ASN | B | 177 | 32.301 | −15.503 | 20.642 | 1.00 | 37.90 | C |
| ATOM | 6628 | O | ASN | B | 177 | 31.866 | −14.500 | 21.194 | 1.00 | 35.57 | O |
| ATOM | 6629 | N | GLY | B | 178 | 31.503 | −16.503 | 20.325 | 1.00 | 39.88 | N |
| ATOM | 6631 | CA | GLY | B | 178 | 30.097 | −16.376 | 20.610 | 1.00 | 42.83 | C |
| ATOM | 6634 | C | GLY | B | 178 | 29.503 | −17.743 | 20.630 | 1.00 | 38.39 | C |
| ATOM | 6635 | O | GLY | B | 178 | 29.955 | −18.584 | 19.849 | 1.00 | 47.34 | O |
| ATOM | 6636 | N | ASN | B | 179 | 28.536 | −17.955 | 21.523 | 1.00 | 41.20 | N |
| ATOM | 6638 | CA | ASN | B | 179 | 27.703 | −19.142 | 21.467 | 1.00 | 38.96 | C |
| ATOM | 6640 | CB | ASN | B | 179 | 28.179 | −20.228 | 22.420 | 1.00 | 50.22 | C |
| ATOM | 6643 | CG | ASN | B | 179 | 28.757 | −19.690 | 23.701 | 1.00 | 57.26 | C |
| ATOM | 6644 | OD1 | ASN | B | 179 | 28.078 | −19.692 | 24.730 | 1.00 | 64.15 | O |
| ATOM | 6645 | ND2 | ASN | B | 179 | 30.047 | −19.346 | 23.680 | 1.00 | 52.38 | N |
| ATOM | 6648 | C | ASN | B | 179 | 26.246 | −18.844 | 21.701 | 1.00 | 42.67 | C |
| ATOM | 6649 | O | ASN | B | 179 | 25.905 | −17.899 | 22.403 | 1.00 | 40.40 | O |
| ATOM | 6650 | N | ILE | B | 180 | 25.385 | −19.617 | 21.043 | 1.00 | 42.06 | N |
| ATOM | 6652 | CA | ILE | B | 180 | 23.953 | −19.417 | 21.180 | 1.00 | 39.48 | C |
| ATOM | 6654 | CB | ILE | B | 180 | 23.303 | −18.858 | 19.878 | 1.00 | 33.35 | C |
| ATOM | 6656 | CG1 | ILE | B | 180 | 23.767 | −17.430 | 19.635 | 1.00 | 37.92 | C |
| ATOM | 6659 | CD1 | ILE | B | 180 | 23.619 | −16.967 | 18.221 | 1.00 | 40.22 | C |
| ATOM | 6663 | CG2 | ILE | B | 180 | 21.784 | −18.807 | 20.039 | 1.00 | 33.59 | C |
| ATOM | 6667 | C | ILE | B | 180 | 23.374 | −20.754 | 21.577 | 1.00 | 35.64 | C |
| ATOM | 6668 | O | ILE | B | 180 | 23.718 | −21.771 | 20.989 | 1.00 | 34.60 | O |
| ATOM | 6669 | N | LYS | B | 181 | 22.632 | −20.751 | 22.674 | 1.00 | 40.92 | N |
| ATOM | 6671 | CA | LYS | B | 181 | 22.098 | −21.967 | 23.257 | 1.00 | 48.20 | C |
| ATOM | 6673 | CB | LYS | B | 181 | 22.641 | −22.145 | 24.689 | 1.00 | 55.98 | C |
| ATOM | 6676 | CG | LYS | B | 181 | 21.930 | −23.230 | 25.529 | 1.00 | 67.43 | C |
| ATOM | 6679 | CD | LYS | B | 181 | 22.423 | −24.666 | 25.240 | 1.00 | 64.71 | C |
| ATOM | 6682 | CE | LYS | B | 181 | 22.482 | −25.528 | 26.526 | 1.00 | 74.58 | C |
| ATOM | 6685 | NZ | LYS | B | 181 | 23.675 | −26.445 | 26.621 | 1.00 | 66.99 | N |
| ATOM | 6689 | C | LYS | B | 181 | 20.572 | −21.894 | 23.246 | 1.00 | 45.02 | C |
| ATOM | 6690 | O | LYS | B | 181 | 20.005 | −21.045 | 23.921 | 1.00 | 44.84 | O |
| ATOM | 6691 | N | LEU | B | 182 | 19.930 | −22.754 | 22.446 | 1.00 | 46.55 | N |
| ATOM | 6693 | CA | LEU | B | 182 | 18.482 | −22.999 | 22.530 | 1.00 | 46.68 | C |
| ATOM | 6695 | CB | LEU | B | 182 | 17.910 | −23.297 | 21.149 | 1.00 | 34.99 | C |
| ATOM | 6698 | CG | LEU | B | 182 | 18.285 | −22.373 | 19.989 | 1.00 | 55.14 | C |
| ATOM | 6700 | CD1 | LEU | B | 182 | 17.235 | −22.520 | 18.895 | 1.00 | 62.46 | C |
| ATOM | 6704 | CD2 | LEU | B | 182 | 18.353 | −20.939 | 20.438 | 1.00 | 60.20 | C |
| ATOM | 6708 | C | LEU | B | 182 | 18.167 | −24.217 | 23.396 | 1.00 | 44.01 | C |
| ATOM | 6709 | O | LEU | B | 182 | 18.567 | −25.324 | 23.069 | 1.00 | 43.05 | O |
| ATOM | 6710 | N | SER | B | 183 | 17.347 | −24.058 | 24.421 | 1.00 | 52.56 | N |
| ATOM | 6712 | CA | SER | B | 183 | 17.064 | −25.200 | 25.283 | 1.00 | 61.03 | C |
| ATOM | 6714 | CB | SER | B | 183 | 17.276 | −24.873 | 26.766 | 1.00 | 57.91 | C |
| ATOM | 6717 | OG | SER | B | 183 | 16.317 | −23.937 | 27.212 | 1.00 | 78.94 | O |
| ATOM | 6719 | C | SER | B | 183 | 15.685 | −25.775 | 25.012 | 1.00 | 61.74 | C |
| ATOM | 6720 | O | SER | B | 183 | 14.802 | −25.086 | 24.504 | 1.00 | 61.06 | O |
| ATOM | 6721 | N | GLN | B | 184 | 15.574 | −27.087 | 25.208 | 1.00 | 73.23 | N |
| ATOM | 6723 | CA | GLN | B | 184 | 14.298 | −27.797 | 25.215 | 1.00 | 73.13 | C |
| ATOM | 6725 | CB | GLN | B | 184 | 14.549 | −29.299 | 25.070 | 1.00 | 74.12 | C |
| ATOM | 6728 | CG | GLN | B | 184 | 14.507 | −29.813 | 23.632 | 1.00 | 78.50 | C |
| ATOM | 6731 | CD | GLN | B | 184 | 14.666 | −31.336 | 23.509 | 1.00 | 86.61 | C |
| ATOM | 6732 | OE1 | GLN | B | 184 | 13.805 | −31.996 | 22.918 | 1.00 | 89.49 | O |
| ATOM | 6733 | NE2 | GLN | B | 184 | 15.776 | −31.884 | 24.020 | 1.00 | 75.80 | N |
| ATOM | 6736 | C | GLN | B | 184 | 13.603 | −27.517 | 26.542 | 1.00 | 76.26 | C |
| ATOM | 6737 | O | GLN | B | 184 | 14.041 | −28.019 | 27.582 | 1.00 | 74.49 | O |
| ATOM | 6738 | N | THR | B | 185 | 12.593 | −26.645 | 26.506 | 1.00 | 80.59 | N |
| ATOM | 6740 | CA | THR | B | 185 | 11.556 | −26.562 | 27.543 | 1.00 | 87.37 | C |
| ATOM | 6742 | CB | THR | B | 185 | 11.070 | −25.084 | 27.747 | 1.00 | 88.07 | C |
| ATOM | 6744 | OG1 | THR | B | 185 | 10.486 | −24.921 | 29.046 | 1.00 | 75.92 | O |
| ATOM | 6746 | CG2 | THR | B | 185 | 9.923 | −24.717 | 26.801 | 1.00 | 86.29 | C |
| ATOM | 6750 | C | THR | B | 185 | 10.383 | −27.497 | 27.184 | 1.00 | 95.75 | C |
| ATOM | 6751 | O | THR | B | 185 | 10.299 | −28.006 | 26.062 | 1.00 | 94.02 | O |
| ATOM | 6752 | N | SER | B | 186 | 9.480 | −27.729 | 28.134 | 1.00 | 104.26 | N |
| ATOM | 6754 | CA | SER | B | 186 | 8.603 | −28.899 | 28.090 | 1.00 | 109.97 | C |
| ATOM | 6756 | CB | SER | B | 186 | 8.005 | −29.142 | 29.475 | 1.00 | 109.97 | C |
| ATOM | 6759 | OG | SER | B | 186 | 9.043 | −29.276 | 30.436 | 1.00 | 107.52 | O |
| ATOM | 6761 | C | SER | B | 186 | 7.526 | −28.821 | 26.999 | 1.00 | 115.83 | C |
| ATOM | 6762 | O | SER | B | 186 | 7.836 | −29.046 | 25.830 | 1.00 | 115.81 | O |
| ATOM | 6763 | N | ASN | B | 187 | 6.272 | −28.538 | 27.357 | 1.00 | 123.32 | N |
| ATOM | 6765 | CA | ASN | B | 187 | 5.238 | −28.245 | 26.352 | 1.00 | 127.89 | C |
| ATOM | 6767 | CB | ASN | B | 187 | 4.456 | −29.510 | 25.938 | 1.00 | 127.21 | C |
| ATOM | 6770 | CG | ASN | B | 187 | 3.781 | −30.223 | 27.111 | 1.00 | 126.02 | C |
| ATOM | 6771 | OD1 | ASN | B | 187 | 3.970 | −31.426 | 27.306 | 1.00 | 119.98 | O |
| ATOM | 6772 | ND2 | ASN | B | 187 | 2.956 | −29.498 | 27.861 | 1.00 | 111.54 | N |
| ATOM | 6775 | C | ASN | B | 187 | 4.279 | −27.108 | 26.732 | 1.00 | 131.37 | C |
| ATOM | 6776 | O | ASN | B | 187 | 4.487 | −26.414 | 27.730 | 1.00 | 131.36 | O |
| ATOM | 6777 | N | VAL | B | 188 | 3.271 | −26.889 | 25.888 | 1.00 | 134.86 | N |
| ATOM | 6779 | CA | VAL | B | 188 | 2.151 | −25.988 | 26.183 | 1.00 | 137.02 | C |
| ATOM | 6781 | CB | VAL | B | 188 | 1.301 | −26.493 | 27.388 | 1.00 | 137.17 | C |
| ATOM | 6783 | CG1 | VAL | B | 188 | 0.046 | −25.630 | 27.576 | 1.00 | 136.44 | C |
| ATOM | 6787 | CG2 | VAL | B | 188 | 0.911 | −27.958 | 27.201 | 1.00 | 134.39 | C |
| ATOM | 6791 | C | VAL | B | 188 | 2.578 | −24.536 | 26.423 | 1.00 | 138.11 | C |
| ATOM | 6792 | O | VAL | B | 188 | 1.902 | −23.793 | 27.139 | 1.00 | 137.92 | O |
| ATOM | 6793 | N | ASP | B | 189 | 3.669 | −24.123 | 25.780 | 1.00 | 139.40 | N |
| ATOM | 6795 | CA | ASP | B | 189 | 4.281 | −22.817 | 26.037 | 1.00 | 139.70 | C |
| ATOM | 6797 | CB | ASP | B | 189 | 5.792 | −22.994 | 26.246 | 1.00 | 139.77 | C |
| ATOM | 6800 | CG | ASP | B | 189 | 6.523 | −21.672 | 26.438 | 1.00 | 138.49 | C |
| ATOM | 6801 | OD1 | ASP | B | 189 | 6.170 | −20.914 | 27.374 | 1.00 | 140.25 | O |
| ATOM | 6802 | OD2 | ASP | B | 189 | 7.476 | −21.319 | 25.707 | 1.00 | 123.28 | O |
| ATOM | 6803 | C | ASP | B | 189 | 4.014 | −21.836 | 24.888 | 1.00 | 138.45 | C |
| ATOM | 6804 | O | ASP | B | 189 | 4.122 | −22.216 | 23.722 | 1.00 | 137.58 | O |
| ATOM | 6805 | N | LYS | B | 190 | 3.700 | −20.582 | 25.224 | 1.00 | 137.68 | N |
| ATOM | 6807 | CA | LYS | B | 190 | 3.292 | −19.558 | 24.247 | 1.00 | 136.66 | C |
| ATOM | 6809 | CB | LYS | B | 190 | 2.817 | −18.281 | 24.965 | 1.00 | 136.46 | C |
| ATOM | 6812 | CG | LYS | B | 190 | 1.694 | −17.515 | 24.249 | 1.00 | 129.76 | C |
| ATOM | 6815 | CD | LYS | B | 190 | 2.230 | −16.524 | 23.199 | 1.00 | 116.02 | C |
| ATOM | 6818 | CE | LYS | B | 190 | 2.791 | −15.243 | 23.860 | 1.00 | 117.29 | C |
| ATOM | 6821 | NZ | LYS | B | 190 | 4.259 | −15.038 | 23.527 | 1.00 | 117.57 | N |
| ATOM | 6825 | C | LYS | B | 190 | 4.377 | −19.186 | 23.223 | 1.00 | 136.34 | C |
| ATOM | 6826 | O | LYS | B | 190 | 4.110 | −19.136 | 22.015 | 1.00 | 137.52 | O |
| ATOM | 6827 | N | GLU | B | 191 | 5.577 | −18.875 | 23.712 | 1.00 | 133.25 | N |
| ATOM | 6829 | CA | GLU | B | 191 | 6.725 | −18.604 | 22.848 | 1.00 | 128.64 | C |
| ATOM | 6831 | CB | GLU | B | 191 | 8.032 | −18.779 | 23.631 | 1.00 | 127.81 | C |
| ATOM | 6834 | CG | GLU | B | 191 | 8.801 | −20.044 | 23.268 | 1.00 | 119.94 | C |
| ATOM | 6837 | CD | GLU | B | 191 | 9.748 | −20.501 | 24.359 | 1.00 | 112.10 | C |
| ATOM | 6838 | OE1 | GLU | B | 191 | 10.860 | −20.966 | 24.022 | 1.00 | 92.53 | O |
| ATOM | 6839 | OE2 | GLU | B | 191 | 9.379 | −20.398 | 25.550 | 1.00 | 99.24 | O |
| ATOM | 6840 | C | GLU | B | 191 | 6.727 | −19.562 | 21.661 | 1.00 | 127.26 | C |
| ATOM | 6841 | O | GLU | B | 191 | 6.566 | −20.775 | 21.831 | 1.00 | 127.86 | O |
| ATOM | 6842 | N | GLU | B | 192 | 6.921 | −19.030 | 20.459 | 1.00 | 123.62 | N |
| ATOM | 6844 | CA | GLU | B | 192 | 7.251 | −19.891 | 19.334 | 1.00 | 120.94 | C |
| ATOM | 6846 | CB | GLU | B | 192 | 6.791 | −19.296 | 18.004 | 1.00 | 120.26 | C |
| ATOM | 6849 | CG | GLU | B | 192 | 5.903 | −20.252 | 17.219 | 1.00 | 122.34 | C |
| ATOM | 6852 | CD | GLU | B | 192 | 4.906 | −20.991 | 18.102 | 1.00 | 124.15 | C |
| ATOM | 6853 | OE1 | GLU | B | 192 | 4.313 | −20.347 | 19.005 | 1.00 | 127.72 | O |
| ATOM | 6854 | OE2 | GLU | B | 192 | 4.711 | −22.218 | 17.898 | 1.00 | 116.50 | O |
| ATOM | 6855 | C | GLU | B | 192 | 8.743 | −20.195 | 19.322 | 1.00 | 117.73 | C |
| ATOM | 6856 | O | GLU | B | 192 | 9.496 | −19.623 | 18.529 | 1.00 | 114.76 | O |
| ATOM | 6857 | N | GLU | B | 193 | 9.144 | −21.079 | 20.237 | 1.00 | 115.51 | N |
| ATOM | 6859 | CA | GLU | B | 193 | 10.451 | −21.742 | 20.210 | 1.00 | 114.51 | C |
| ATOM | 6861 | CB | GLU | B | 193 | 11.527 | −20.836 | 20.830 | 1.00 | 114.52 | C |
| ATOM | 6864 | CG | GLU | B | 193 | 12.743 | −20.588 | 19.946 | 1.00 | 113.77 | C |
| ATOM | 6867 | CD | GLU | B | 193 | 12.841 | −19.146 | 19.472 | 1.00 | 117.35 | C |
| ATOM | 6868 | OE1 | GLU | B | 193 | 12.083 | −18.755 | 18.552 | 1.00 | 105.71 | O |
| ATOM | 6869 | OE2 | GLU | B | 193 | 13.684 | −18.398 | 20.016 | 1.00 | 116.16 | O |
| ATOM | 6870 | C | GLU | B | 193 | 10.399 | −23.098 | 20.939 | 1.00 | 110.97 | C |
| ATOM | 6871 | O | GLU | B | 193 | 10.905 | −23.233 | 22.057 | 1.00 | 113.07 | O |
| ATOM | 6872 | N | ALA | B | 194 | 9.785 | −24.100 | 20.310 | 1.00 | 105.82 | N |
| ATOM | 6874 | CA | ALA | B | 194 | 9.806 | −25.464 | 20.843 | 1.00 | 102.73 | C |
| ATOM | 6876 | CB | ALA | B | 194 | 8.403 | −26.079 | 20.824 | 1.00 | 101.54 | C |
| ATOM | 6880 | C | ALA | B | 194 | 10.793 | −26.369 | 20.094 | 1.00 | 98.32 | C |
| ATOM | 6881 | O | ALA | B | 194 | 10.459 | −27.512 | 19.771 | 1.00 | 101.54 | O |
| ATOM | 6882 | N | VAL | B | 195 | 11.988 | −25.848 | 19.803 | 1.00 | 88.44 | N |
| ATOM | 6884 | CA | VAL | B | 195 | 13.147 | −26.665 | 19.441 | 1.00 | 75.58 | C |
| ATOM | 6886 | CB | VAL | B | 195 | 14.419 | −26.073 | 20.055 | 1.00 | 74.31 | C |
| ATOM | 6888 | CG1 | VAL | B | 195 | 15.598 | −27.036 | 19.925 | 1.00 | 71.70 | C |
| ATOM | 6892 | CG2 | VAL | B | 195 | 14.721 | −24.715 | 19.456 | 1.00 | 80.11 | C |
| ATOM | 6896 | C | VAL | B | 195 | 13.026 | −28.079 | 19.997 | 1.00 | 69.46 | C |
| ATOM | 6897 | O | VAL | B | 195 | 13.095 | −28.254 | 21.211 | 1.00 | 69.88 | O |
| ATOM | 6898 | N | THR | B | 196 | 12.875 | −29.071 | 19.118 | 1.00 | 63.37 | N |
| ATOM | 6900 | CA | THR | B | 196 | 12.850 | −30.484 | 19.497 | 1.00 | 57.22 | C |
| ATOM | 6902 | CB | THR | B | 196 | 11.487 | −31.141 | 19.124 | 1.00 | 59.82 | C |
| ATOM | 6904 | OG1 | THR | B | 196 | 11.464 | −31.539 | 17.748 | 1.00 | 67.95 | O |
| ATOM | 6906 | CG2 | THR | B | 196 | 10.337 | −30.136 | 19.221 | 1.00 | 63.37 | C |
| ATOM | 6910 | C | THR | B | 196 | 13.990 | −31.257 | 18.859 | 1.00 | 55.59 | C |
| ATOM | 6911 | O | THR | B | 196 | 14.395 | −30.974 | 17.733 | 1.00 | 54.95 | O |
| ATOM | 6912 | N | ILE | B | 197 | 14.502 | −32.260 | 19.566 | 1.00 | 57.21 | N |
| ATOM | 6914 | CA | ILE | B | 197 | 15.649 | −33.011 | 19.069 | 1.00 | 58.99 | C |
| ATOM | 6916 | CB | ILE | B | 197 | 16.981 | −32.336 | 19.565 | 1.00 | 61.15 | C |
| ATOM | 6918 | CG1 | ILE | B | 197 | 18.142 | −33.313 | 19.721 | 1.00 | 57.14 | C |
| ATOM | 6921 | CD1 | ILE | B | 197 | 18.535 | −34.013 | 18.476 | 1.00 | 76.20 | C |
| ATOM | 6925 | CG2 | ILE | B | 197 | 16.780 | −31.565 | 20.873 | 1.00 | 69.09 | C |
| ATOM | 6929 | C | ILE | B | 197 | 15.533 | −34.545 | 19.269 | 1.00 | 63.65 | C |
| ATOM | 6930 | O | ILE | B | 197 | 15.207 | −35.059 | 20.350 | 1.00 | 63.40 | O |
| ATOM | 6931 | N | GLU | B | 198 | 15.662 | −35.270 | 18.164 | 1.00 | 59.66 | N |
| ATOM | 6933 | CA | GLU | B | 198 | 15.607 | −36.720 | 18.203 | 1.00 | 61.01 | C |
| ATOM | 6935 | CB | GLU | B | 198 | 14.622 | −37.287 | 17.161 | 1.00 | 53.02 | C |
| ATOM | 6938 | CG | GLU | B | 198 | 14.729 | −38.808 | 16.996 | 1.00 | 76.32 | C |
| ATOM | 6941 | CD | GLU | B | 198 | 13.421 | −39.531 | 16.645 | 1.00 | 93.86 | C |
| ATOM | 6942 | OE1 | GLU | B | 198 | 13.319 | −40.757 | 16.919 | 1.00 | 97.31 | O |
| ATOM | 6943 | OE2 | GLU | B | 198 | 12.501 | −38.907 | 16.062 | 1.00 | 88.67 | O |
| ATOM | 6944 | C | GLU | B | 198 | 17.033 | −37.185 | 17.944 | 1.00 | 62.82 | C |
| ATOM | 6945 | O | GLU | B | 198 | 17.608 | −36.847 | 16.902 | 1.00 | 57.40 | O |
| ATOM | 6946 | N | MET | B | 199 | 17.585 | −37.958 | 18.886 | 1.00 | 66.00 | N |
| ATOM | 6948 | CA | MET | B | 199 | 19.040 | −38.066 | 19.080 | 1.00 | 67.91 | C |
| ATOM | 6950 | CB | MET | B | 199 | 19.490 | −37.167 | 20.240 | 1.00 | 63.02 | C |
| ATOM | 6953 | CG | MET | B | 199 | 20.953 | −36.760 | 20.114 | 1.00 | 74.36 | C |
| ATOM | 6956 | SD | MET | B | 199 | 21.773 | −36.364 | 21.652 | 1.00 | 85.24 | S |
| ATOM | 6957 | CE | MET | B | 199 | 22.005 | −38.046 | 22.375 | 1.00 | 84.66 | C |
| ATOM | 6961 | C | MET | B | 199 | 19.534 | −39.478 | 19.380 | 1.00 | 61.12 | C |
| ATOM | 6962 | O | MET | B | 199 | 19.039 | −40.113 | 20.295 | 1.00 | 67.71 | O |
| ATOM | 6963 | N | ASN | B | 200 | 20.542 | −39.942 | 18.649 | 1.00 | 60.23 | N |
| ATOM | 6965 | CA | ASN | B | 200 | 21.253 | −41.179 | 18.995 | 1.00 | 64.18 | C |
| ATOM | 6967 | CB | ASN | B | 200 | 21.573 | −42.002 | 17.741 | 1.00 | 63.00 | C |
| ATOM | 6970 | CG | ASN | B | 200 | 20.332 | −42.401 | 16.984 | 1.00 | 73.95 | C |
| ATOM | 6971 | OD1 | ASN | B | 200 | 19.210 | −42.310 | 17.507 | 1.00 | 71.63 | O |
| ATOM | 6972 | ND2 | ASN | B | 200 | 20.515 | −42.807 | 15.732 | 1.00 | 76.53 | N |
| ATOM | 6975 | C | ASN | B | 200 | 22.543 | −40.968 | 19.782 | 1.00 | 61.59 | C |
| ATOM | 6976 | O | ASN | B | 200 | 22.874 | −41.752 | 20.664 | 1.00 | 60.44 | O |
| ATOM | 6977 | N | GLU | B | 201 | 23.307 | −39.949 | 19.426 | 1.00 | 62.80 | N |
| ATOM | 6979 | CA | GLU | B | 201 | 24.514 | −39.629 | 20.179 | 1.00 | 61.46 | C |
| ATOM | 6981 | CB | GLU | B | 201 | 25.662 | −40.576 | 19.818 | 1.00 | 64.73 | C |
| ATOM | 6984 | CG | GLU | B | 201 | 26.209 | −40.401 | 18.408 | 1.00 | 76.26 | C |
| ATOM | 6987 | CD | GLU | B | 201 | 26.844 | −41.660 | 17.835 | 1.00 | 85.86 | C |
| ATOM | 6988 | OE1 | GLU | B | 201 | 26.175 | −42.378 | 17.057 | 1.00 | 97.13 | O |
| ATOM | 6989 | OE2 | GLU | B | 201 | 28.024 | −41.922 | 18.135 | 1.00 | 89.55 | O |
| ATOM | 6998 | C | GLU | B | 201 | 24.891 | −38.187 | 19.881 | 1.00 | 57.95 | C |
| ATOM | 6991 | O | GLU | B | 201 | 24.666 | −37.698 | 18.771 | 1.00 | 56.10 | O |
| ATOM | 6992 | N | PRO | B | 202 | 25.353 | −37.478 | 20.904 | 1.00 | 54.52 | N |
| ATOM | 6993 | CA | PRO | B | 202 | 25.850 | −36.119 | 20.733 | 1.00 | 59.49 | C |
| ATOM | 6995 | CB | PRO | B | 202 | 26.688 | −35.898 | 21.991 | 1.00 | 60.60 | C |
| ATOM | 6998 | CG | PRO | B | 202 | 26.008 | −36.702 | 23.007 | 1.00 | 64.35 | C |
| ATOM | 7001 | CD | PRO | B | 202 | 25.413 | −37.902 | 22.310 | 1.00 | 55.78 | C |
| ATOM | 7004 | C | PRO | B | 202 | 26.722 | −36.010 | 19.502 | 1.00 | 63.26 | C |
| ATOM | 7005 | O | PRO | B | 202 | 27.569 | −36.888 | 19.262 | 1.00 | 64.70 | O |
| ATOM | 7006 | N | VAL | B | 203 | 26.485 | −34.966 | 18.714 | 1.00 | 61.81 | N |
| ATOM | 7008 | CA | VAL | B | 203 | 27.360 | −34.675 | 17.593 | 1.00 | 62.13 | C |
| ATOM | 7010 | CB | VAL | B | 203 | 26.628 | −34.722 | 16.254 | 1.00 | 61.09 | C |
| ATOM | 7012 | CG1 | VAL | B | 203 | 27.589 | −35.208 | 15.186 | 1.00 | 70.63 | C |
| ATOM | 7016 | CG2 | VAL | B | 203 | 25.393 | −35.620 | 16.334 | 1.00 | 69.07 | C |
| ATOM | 7020 | C | VAL | B | 203 | 27.958 | −33.300 | 17.749 | 1.00 | 61.15 | C |
| ATOM | 7021 | O | VAL | B | 203 | 27.435 | −32.472 | 18.500 | 1.00 | 63.14 | O |
| ATOM | 7022 | N | GLN | B | 204 | 29.015 | −33.055 | 16.984 | 1.00 | 55.78 | N |
| ATOM | 7024 | CA | GLN | B | 204 | 29.696 | −31.777 | 17.013 | 1.00 | 50.61 | C |
| ATOM | 7026 | CB | GLN | B | 204 | 30.665 | −31.767 | 18.180 | 1.00 | 54.87 | C |
| ATOM | 7029 | CG | GLN | B | 204 | 30.931 | −30.412 | 18.776 | 1.00 | 68.37 | C |
| ATOM | 7032 | CD | GLN | B | 204 | 32.422 | −30.159 | 18.979 | 1.00 | 77.41 | C |
| ATOM | 7033 | OE1 | GLN | B | 204 | 32.934 | −30.249 | 20.100 | 1.00 | 86.57 | O |
| ATOM | 7034 | NE2 | GLN | B | 204 | 33.120 | −29.850 | 17.891 | 1.00 | 70.08 | N |
| ATOM | 7037 | C | GLN | B | 204 | 30.434 | −31.601 | 15.690 | 1.00 | 54.46 | C |
| ATOM | 7038 | O | GLN | B | 204 | 31.437 | −32.274 | 15.450 | 1.00 | 60.97 | O |
| ATOM | 7039 | N | LEU | B | 205 | 29.904 | −30.736 | 14.821 | 1.00 | 45.74 | N |
| ATOM | 7041 | CA | LEU | B | 205 | 30.427 | −30.555 | 13.469 | 1.00 | 44.31 | C |
| ATOM | 7043 | CB | LEU | B | 205 | 29.528 | −31.237 | 12.426 | 1.00 | 40.15 | C |
| ATOM | 7046 | CG | LEU | B | 205 | 29.519 | −32.750 | 12.681 | 1.00 | 49.96 | C |
| ATOM | 7048 | CD1 | LEU | B | 205 | 28.277 | −33.472 | 12.147 | 1.00 | 51.40 | C |
| ATOM | 7052 | CD2 | LEU | B | 205 | 30.817 | −33.381 | 12.173 | 1.00 | 59.40 | C |
| ATOM | 7056 | C | LEU | B | 205 | 30.523 | −29.078 | 13.185 | 1.00 | 40.18 | C |
| ATOM | 7057 | O | LEU | B | 205 | 29.743 | −28.309 | 13.728 | 1.00 | 42.28 | O |
| ATOM | 7058 | N | THR | B | 206 | 31.498 | −28.707 | 12.359 | 1.00 | 33.64 | N |
| ATOM | 7060 | CA | THR | B | 206 | 31.687 | −27.346 | 11.853 | 1.00 | 32.24 | C |
| ATOM | 7062 | CB | THR | B | 206 | 33.187 | −27.012 | 11.947 | 1.00 | 33.24 | C |
| ATOM | 7064 | OG1 | THR | B | 206 | 33.619 | −27.251 | 13.282 | 1.00 | 46.43 | O |
| ATOM | 7066 | CG2 | THR | B | 206 | 33.521 | −25.549 | 11.672 | 1.00 | 40.95 | C |
| ATOM | 7070 | C | THR | B | 206 | 31.241 | −27.229 | 10.404 | 1.00 | 37.22 | C |
| ATOM | 7071 | O | THR | B | 206 | 31.387 | −28.176 | 9.617 | 1.00 | 42.48 | O |
| ATOM | 7072 | N | PHE | B | 207 | 30.756 | −26.035 | 10.059 | 1.00 | 37.33 | N |
| ATOM | 7074 | CA | PHE | B | 207 | 30.100 | −25.756 | 8.778 | 1.00 | 31.79 | C |
| ATOM | 7076 | CB | PHE | B | 207 | 28.582 | −25.881 | 8.927 | 1.00 | 28.10 | C |
| ATOM | 7079 | CG | PHE | B | 207 | 28.119 | −27.286 | 9.172 | 1.00 | 29.38 | C |
| ATOM | 7080 | CD1 | PHE | B | 207 | 28.181 | −28.230 | 8.172 | 1.00 | 35.44 | C |
| ATOM | 7082 | CE1 | PHE | B | 207 | 27.746 | −29.546 | 8.391 | 1.00 | 31.81 | C |
| ATOM | 7084 | CZ | PHE | B | 207 | 27.304 | −29.901 | 9.638 | 1.00 | 30.92 | C |
| ATOM | 7086 | CE2 | PHE | B | 207 | 27.292 | −28.963 | 10.658 | 1.00 | 29.21 | C |
| ATOM | 7088 | CD2 | PHE | B | 207 | 27.720 | −27.688 | 10.431 | 1.00 | 32.05 | C |
| ATOM | 7090 | C | PHE | B | 207 | 30.429 | −24.350 | 8.337 | 1.00 | 29.29 | C |
| ATOM | 7091 | O | PHE | B | 207 | 30.508 | −23.481 | 9.188 | 1.00 | 42.35 | O |
| ATOM | 7092 | N | ALA | B | 208 | 30.731 | −24.134 | 7.050 | 1.00 | 33.64 | N |
| ATOM | 7094 | CA | ALA | B | 208 | 30.876 | −22.787 | 6.513 | 1.00 | 35.01 | C |
| ATOM | 7096 | CB | ALA | B | 208 | 31.348 | −22.851 | 5.095 | 1.00 | 36.14 | C |
| ATOM | 7100 | C | ALA | B | 208 | 29.603 | −21.932 | 6.573 | 1.00 | 38.54 | C |
| ATOM | 7101 | O | ALA | B | 208 | 28.569 | −22.296 | 5.977 | 1.00 | 36.59 | O |
| ATOM | 7102 | N | LEU | B | 209 | 29.690 | −20.770 | 7.226 | 1.00 | 36.15 | N |
| ATOM | 7104 | CA | LEU | B | 209 | 28.503 | −19.929 | 7.434 | 1.00 | 39.75 | C |
| ATOM | 7106 | CB | LEU | B | 209 | 28.714 | −18.893 | 8.546 | 1.00 | 40.25 | C |
| ATOM | 7109 | CG | LEU | B | 209 | 28.413 | −19.322 | 9.981 | 1.00 | 31.84 | C |
| ATOM | 7111 | CD1 | LEU | B | 209 | 29.162 | −18.446 | 11.001 | 1.00 | 43.34 | C |
| ATOM | 7115 | CD2 | LEU | B | 209 | 26.958 | −19.292 | 10.301 | 1.00 | 31.76 | C |
| ATOM | 7119 | C | LEU | B | 209 | 28.027 | −19.235 | 6.149 | 1.00 | 41.05 | C |
| ATOM | 7120 | O | LEU | B | 209 | 26.843 | −18.975 | 5.965 | 1.00 | 32.08 | O |
| ATOM | 7121 | N | ARG | B | 210 | 28.936 | −18.992 | 5.220 | 1.00 | 33.64 | N |
| ATOM | 7123 | CA | ARG | B | 210 | 28.567 | −18.433 | 3.918 | 1.00 | 34.81 | C |
| ATOM | 7125 | CB | ARG | B | 210 | 29.830 | −18.388 | 3.048 | 1.00 | 41.87 | C |
| ATOM | 7128 | CG | ARG | B | 210 | 29.618 | −18.008 | 1.581 | 1.00 | 45.06 | C |
| ATOM | 7131 | CD | ARG | B | 210 | 30.947 | −18.051 | 0.784 | 1.00 | 57.83 | C |
| ATOM | 7134 | NE | ARG | B | 210 | 30.700 | −18.085 | −0.659 | 1.00 | 70.00 | N |
| ATOM | 7136 | CZ | ARG | B | 210 | 30.266 | −17.050 | −1.368 | 1.00 | 56.14 | C |
| ATOM | 7137 | NH1 | ARG | B | 210 | 30.106 | −15.872 | −0.794 | 1.00 | 58.38 | N |
| ATOM | 7140 | NH2 | ARG | B | 210 | 30.004 | −17.190 | −2.661 | 1.00 | 75.35 | N |
| ATOM | 7143 | C | ARG | B | 210 | 27.453 | −19.247 | 3.233 | 1.00 | 34.70 | C |
| ATOM | 7144 | O | ARG | B | 210 | 26.507 | −18.713 | 2.673 | 1.00 | 29.59 | O |
| ATOM | 7145 | N | TYR | B | 211 | 27.555 | −20.562 | 3.287 | 1.00 | 25.07 | N |
| ATOM | 7147 | CA | TYR | B | 211 | 26.625 | −21.382 | 2.571 | 1.00 | 26.04 | C |
| ATOM | 7149 | CB | TYR | B | 211 | 27.294 | −22.716 | 2.237 | 1.00 | 24.11 | C |
| ATOM | 7152 | CG | TYR | B | 211 | 28.226 | −22.492 | 1.068 | 1.00 | 35.59 | C |
| ATOM | 7153 | CD1 | TYR | B | 211 | 29.535 | −22.065 | 1.283 | 1.00 | 44.29 | C |
| ATOM | 7155 | CE1 | TYR | B | 211 | 30.402 | −21.830 | 0.224 | 1.00 | 55.07 | C |
| ATOM | 7157 | CZ | TYR | B | 211 | 29.964 | −21.940 | −1.072 | 1.00 | 52.47 | C |
| ATOM | 7158 | OH | TYR | B | 211 | 30.867 | −21.670 | −2.086 | 1.00 | 74.05 | O |
| ATOM | 7160 | CE2 | TYR | B | 211 | 28.666 | −22.351 | −1.327 | 1.00 | 55.75 | C |
| ATOM | 7162 | CD2 | TYR | B | 211 | 27.789 | −22.614 | −0.247 | 1.00 | 35.03 | C |
| ATOM | 7164 | C | TYR | B | 211 | 25.319 | −21.543 | 3.349 | 1.00 | 27.06 | C |
| ATOM | 7165 | O | TYR | B | 211 | 24.260 | −21.554 | 2.741 | 1.00 | 27.71 | O |
| ATOM | 7166 | N | LEU | B | 212 | 25.404 | −21.722 | 4.667 | 1.00 | 25.14 | N |
| ATOM | 7168 | CA | LEU | B | 212 | 24.222 | −21.703 | 5.533 | 1.00 | 32.37 | C |
| ATOM | 7170 | CB | LEU | B | 212 | 24.609 | −21.814 | 7.001 | 1.00 | 27.86 | C |
| ATOM | 7173 | CG | LEU | B | 212 | 25.166 | −23.179 | 7.343 | 1.00 | 28.55 | C |
| ATOM | 7175 | CD1 | LEU | B | 212 | 25.389 | −23.225 | 8.850 | 1.00 | 36.28 | C |
| ATOM | 7179 | CD2 | LEU | B | 212 | 24.213 | −24.321 | 6.862 | 1.00 | 23.80 | C |
| ATOM | 7183 | C | LEU | B | 212 | 23.369 | −20.459 | 5.322 | 1.00 | 25.73 | C |
| ATOM | 7184 | O | LEU | B | 212 | 22.134 | −20.563 | 5.337 | 1.00 | 28.25 | O |
| ATOM | 7185 | N | ASN | B | 213 | 24.026 | −19.331 | 5.031 | 1.00 | 25.66 | N |
| ATOM | 7187 | CA | ASN | B | 213 | 23.369 | −18.047 | 4.756 | 1.00 | 30.27 | C |
| ATOM | 7189 | CB | ASN | B | 213 | 24.299 | −16.847 | 4.997 | 1.00 | 31.42 | C |
| ATOM | 7192 | CG | ASN | B | 213 | 24.464 | −16.553 | 6.473 | 1.00 | 41.07 | C |
| ATOM | 7193 | OD1 | ASN | B | 213 | 23.486 | −16.623 | 7.206 | 1.00 | 40.50 | O |
| ATOM | 7194 | ND2 | ASN | B | 213 | 25.695 | −16.311 | 6.936 | 1.00 | 46.44 | N |
| ATOM | 7197 | C | ASN | B | 213 | 22.758 | −17.979 | 3.365 | 1.00 | 32.34 | C |
| ATOM | 7198 | O | ASN | B | 213 | 21.729 | −17.328 | 3.185 | 1.00 | 35.30 | O |
| ATOM | 7199 | N | PHE | B | 214 | 23.248 | −18.808 | 2.448 | 1.00 | 25.24 | N |
| ATOM | 7201 | CA | PHE | B | 214 | 22.484 | −19.080 | 1.241 | 1.00 | 27.93 | C |
| ATOM | 7203 | CB | PHE | B | 214 | 23.338 | −19.702 | 0.129 | 1.00 | 33.48 | C |
| ATOM | 7206 | CG | PHE | B | 214 | 24.293 | −18.718 | −0.523 | 1.00 | 32.61 | C |
| ATOM | 7207 | CD1 | PHE | B | 214 | 23.854 | −17.474 | −0.970 | 1.00 | 56.79 | C |
| ATOM | 7209 | CE1 | PHE | B | 214 | 24.762 | −16.500 | −1.434 | 1.00 | 45.04 | C |
| ATOM | 7211 | CZ | PHE | B | 214 | 26.107 | −16.798 | −1.498 | 1.00 | 52.73 | C |
| ATOM | 7213 | CE2 | PHE | B | 214 | 26.559 | −18.033 | −1.044 | 1.00 | 53.39 | C |
| ATOM | 7215 | CD2 | PHE | B | 214 | 25.655 | −18.967 | −0.531 | 1.00 | 45.76 | C |
| ATOM | 7217 | C | PHE | B | 214 | 21.197 | −19.870 | 1.506 | 1.00 | 24.15 | C |
| ATOM | 7218 | O | PHE | B | 214 | 20.178 | −19.567 | 0.915 | 1.00 | 30.48 | O |
| ATOM | 7219 | N | PHE | B | 215 | 21.243 | −20.851 | 2.395 | 1.00 | 24.64 | N |
| ATOM | 7221 | CA | PHE | B | 215 | 20.169 | −21.818 | 2.526 | 1.00 | 21.37 | C |
| ATOM | 7223 | CB | PHE | B | 215 | 20.561 | −23.005 | 3.380 | 1.00 | 25.74 | C |
| ATOM | 7226 | CG | PHE | B | 215 | 21.738 | −23.801 | 2.857 | 1.00 | 27.89 | C |
| ATOM | 7227 | CD1 | PHE | B | 215 | 22.268 | −23.557 | 1.617 | 1.00 | 25.49 | C |
| ATOM | 7229 | CE1 | PHE | B | 215 | 23.274 | −24.338 | 1.104 | 1.00 | 29.13 | C |
| ATOM | 7231 | CZ | PHE | B | 215 | 23.873 | −25.296 | 1.894 | 1.00 | 28.18 | C |
| ATOM | 7233 | CE2 | PHE | B | 215 | 23.419 | −25.509 | 3.166 | 1.00 | 38.72 | C |
| ATOM | 7235 | CD2 | PHE | B | 215 | 22.312 | −24.788 | 3.638 | 1.00 | 29.70 | C |
| ATOM | 7237 | C | PHE | B | 215 | 18.990 | −21.196 | 3.252 | 1.00 | 25.20 | C |
| ATOM | 7238 | O | PHE | B | 215 | 17.886 | −21.657 | 3.033 | 1.00 | 30.78 | O |
| ATOM | 7239 | N | THR | B | 216 | 19.235 | −20.189 | 4.100 | 1.00 | 30.45 | N |
| ATOM | 7241 | CA | THR | B | 216 | 18.195 | −19.444 | 4.802 | 1.00 | 31.01 | C |
| ATOM | 7243 | CB | THR | B | 216 | 18.711 | −18.699 | 6.036 | 1.00 | 28.76 | C |
| ATOM | 7245 | OG1 | THR | B | 216 | 19.665 | −17.688 | 5.660 | 1.00 | 34.91 | O |
| ATOM | 7247 | CG2 | THR | B | 216 | 19.366 | −19.691 | 7.003 | 1.00 | 22.05 | C |
| ATOM | 7251 | C | THR | B | 216 | 17.368 | −18.462 | 3.993 | 1.00 | 29.41 | C |
| ATOM | 7252 | O | THR | B | 216 | 16.422 | −17.881 | 4.534 | 1.00 | 22.30 | O |
| ATOM | 7253 | N | LYS | B | 217 | 17.773 | −18.194 | 2.761 | 1.00 | 25.28 | N |
| ATOM | 7255 | CA | LYS | B | 217 | 16.879 | −17.547 | 1.796 | 1.00 | 29.35 | C |
| ATOM | 7257 | CB | LYS | B | 217 | 17.656 | −17.421 | 0.485 | 1.00 | 30.82 | C |
| ATOM | 7260 | CG | LYS | B | 217 | 18.603 | −16.199 | 0.513 | 1.00 | 35.22 | C |
| ATOM | 7263 | CD | LYS | B | 217 | 19.342 | −16.002 | −0.800 | 1.00 | 35.75 | C |
| ATOM | 7266 | CE | LYS | B | 217 | 20.070 | −14.636 | −0.809 | 1.00 | 70.00 | C |
| ATOM | 7269 | NZ | LYS | B | 217 | 21.226 | −14.517 | −1.773 | 1.00 | 63.46 | N |
| ATOM | 7273 | C | LYS | B | 217 | 15.519 | −18.294 | 1.549 | 1.00 | 23.43 | C |
| ATOM | 7274 | O | LYS | B | 217 | 14.589 | −17.729 | 0.979 | 1.00 | 27.09 | O |
| ATOM | 7275 | N | ALA | B | 218 | 15.458 | −19.574 | 1.915 | 1.00 | 25.71 | N |
| ATOM | 7277 | CA | ALA | B | 218 | 14.253 | −20.394 | 1.870 | 1.00 | 21.53 | C |
| ATOM | 7279 | CB | ALA | B | 218 | 14.639 | −21.829 | 1.858 | 1.00 | 14.53 | C |
| ATOM | 7283 | C | ALA | B | 218 | 13.289 | −20.167 | 3.031 | 1.00 | 23.46 | C |
| ATOM | 7284 | O | ALA | B | 218 | 12.299 | −20.881 | 3.130 | 1.00 | 22.77 | O |
| ATOM | 7285 | N | THR | B | 219 | 13.679 | −19.278 | 3.953 | 1.00 | 24.23 | N |
| ATOM | 7287 | CA | THR | B | 219 | 12.967 | −19.046 | 5.187 | 1.00 | 26.67 | C |
| ATOM | 7289 | CB | THR | B | 219 | 13.618 | −17.990 | 6.017 | 1.00 | 24.60 | C |
| ATOM | 7291 | OG1 | THR | B | 219 | 14.894 | −18.490 | 6.484 | 1.00 | 30.80 | O |
| ATOM | 7293 | CG2 | THR | B | 219 | 12.768 | −17.791 | 7.292 | 1.00 | 28.36 | C |
| ATOM | 7297 | C | THR | B | 219 | 11.465 | −18.812 | 5.051 | 1.00 | 24.26 | C |
| ATOM | 7298 | O | THR | B | 219 | 10.712 | −19.512 | 5.689 | 1.00 | 26.89 | O |
| ATOM | 7299 | N | PRO | B | 220 | 11.047 | −17.980 | 4.118 | 1.00 | 27.03 | N |
| ATOM | 7300 | CA | PRO | B | 220 | 9.615 | −17.781 | 3.805 | 1.00 | 36.07 | C |
| ATOM | 7302 | CB | PRO | B | 220 | 9.622 | −16.721 | 2.685 | 1.00 | 29.87 | C |
| ATOM | 7305 | CG | PRO | B | 220 | 10.993 | −16.099 | 2.707 | 1.00 | 35.37 | C |
| ATOM | 7308 | CD | PRO | B | 220 | 11.935 | −17.097 | 3.349 | 1.00 | 28.31 | C |
| ATOM | 7311 | C | PRO | B | 220 | 8.817 | −19.020 | 3.346 | 1.00 | 36.74 | C |
| ATOM | 7312 | O | PRO | B | 220 | 7.599 | −18.990 | 3.373 | 1.00 | 38.45 | O |
| ATOM | 7313 | N | LEU | B | 221 | 9.462 | −20.137 | 3.032 | 1.00 | 37.60 | N |
| ATOM | 7315 | CA | LEU | B | 221 | 8.739 | −21.256 | 2.431 | 1.00 | 27.26 | C |
| ATOM | 7317 | CB | LEU | B | 221 | 9.666 | −22.097 | 1.569 | 1.00 | 34.10 | C |
| ATOM | 7320 | CG | LEU | B | 221 | 10.021 | −21.556 | 0.171 | 1.00 | 25.56 | C |
| ATOM | 7322 | CD1 | LEU | B | 221 | 11.203 | −22.361 | −0.314 | 1.00 | 24.95 | C |
| ATOM | 7326 | CD2 | LEU | B | 221 | 8.913 | −21.704 | −0.805 | 1.00 | 41.35 | C |
| ATOM | 7330 | C | LEU | B | 221 | 8.104 | −22.119 | 3.511 | 1.00 | 32.93 | C |
| ATOM | 7331 | O | LEU | B | 221 | 7.196 | −22.946 | 3.263 | 1.00 | 27.25 | O |
| ATOM | 7332 | N | SER | B | 222 | 8.633 | −21.959 | 4.717 | 1.00 | 31.14 | N |
| ATOM | 7334 | CA | SER | B | 222 | 8.198 | −22.780 | 5.826 | 1.00 | 26.65 | C |
| ATOM | 7336 | CB | SER | B | 222 | 8.869 | −24.141 | 5.819 | 1.00 | 33.47 | C |
| ATOM | 7339 | OG | SER | B | 222 | 8.379 | −24.914 | 6.894 | 1.00 | 27.58 | O |
| ATOM | 7341 | C | SER | B | 222 | 8.612 | −22.068 | 7.075 | 1.00 | 33.25 | C |
| ATOM | 7342 | O | SER | B | 222 | 9.656 | −21.399 | 7.067 | 1.00 | 23.00 | O |
| ATOM | 7343 | N | SER | B | 223 | 7.864 | −22.328 | 8.152 | 1.00 | 25.52 | N |
| ATOM | 7345 | CA | SER | B | 223 | 8.133 | −21.774 | 9.479 | 1.00 | 29.87 | C |
| ATOM | 7347 | CB | SER | B | 223 | 6.829 | −21.553 | 10.245 | 1.00 | 36.89 | C |
| ATOM | 7350 | OG | SER | B | 223 | 6.201 | −22.794 | 10.536 | 1.00 | 29.61 | O |
| ATOM | 7352 | C | SER | B | 223 | 9.026 | −22.613 | 10.345 | 1.00 | 30.69 | C |
| ATOM | 7353 | O | SER | B | 223 | 9.408 | −22.202 | 11.447 | 1.00 | 25.61 | O |
| ATOM | 7354 | N | THR | B | 224 | 9.278 | −23.842 | 9.929 | 1.00 | 32.29 | N |
| ATOM | 7356 | CA | THR | B | 224 | 10.252 | −24.663 | 10.640 | 1.00 | 33.42 | C |
| ATOM | 7358 | CB | THR | B | 224 | 9.543 | −25.892 | 11.244 | 1.00 | 40.72 | C |
| ATOM | 7360 | OG1 | THR | B | 224 | 8.415 | −26.191 | 10.435 | 1.00 | 57.53 | O |
| ATOM | 7362 | CG2 | THR | B | 224 | 8.858 | −25.555 | 12.548 | 1.00 | 53.89 | C |
| ATOM | 7366 | C | THR | B | 224 | 11.317 | −25.159 | 9.671 | 1.00 | 27.19 | C |
| ATOM | 7367 | O | THR | B | 224 | 11.055 | −25.265 | 8.478 | 1.00 | 25.57 | O |
| ATOM | 7368 | N | VAL | B | 225 | 12.489 | −25.502 | 10.212 | 1.00 | 31.40 | N |
| ATOM | 7370 | CA | VAL | B | 225 | 13.588 | −26.079 | 9.452 | 1.00 | 27.92 | C |
| ATOM | 7372 | CB | VAL | B | 225 | 14.712 | −25.022 | 9.198 | 1.00 | 27.52 | C |
| ATOM | 7374 | CG1 | VAL | B | 225 | 15.365 | −24.578 | 10.466 | 1.00 | 25.72 | C |
| ATOM | 7378 | CG2 | VAL | B | 225 | 15.803 | −25.525 | 8.247 | 1.00 | 24.16 | C |
| ATOM | 7382 | C | VAL | B | 225 | 14.061 | −27.272 | 10.285 | 1.00 | 28.15 | C |
| ATOM | 7383 | O | VAL | B | 225 | 14.005 | −27.241 | 11.518 | 1.00 | 35.80 | O |
| ATOM | 7384 | N | THR | B | 226 | 14.492 | −28.322 | 9.601 | 1.00 | 24.70 | N |
| ATOM | 7386 | CA | THR | B | 226 | 14.955 | −29.518 | 10.230 | 1.00 | 23.12 | C |
| ATOM | 7388 | CB | THR | B | 226 | 14.041 | −30.732 | 9.850 | 1.00 | 27.95 | C |
| ATOM | 7390 | OG1 | THR | B | 226 | 14.125 | −30.966 | 8.456 | 1.00 | 47.36 | O |
| ATOM | 7392 | CG2 | THR | B | 226 | 12.556 | −30.427 | 10.011 | 1.00 | 38.78 | C |
| ATOM | 7396 | C | THR | B | 226 | 16.403 | −29.672 | 9.742 | 1.00 | 32.38 | C |
| ATOM | 7397 | O | THR | B | 226 | 16.680 | −29.613 | 8.553 | 1.00 | 37.89 | O |
| ATOM | 7398 | N | LEU | B | 227 | 17.323 | −29.813 | 10.691 | 1.00 | 31.58 | N |
| ATOM | 7400 | CA | LEU | B | 227 | 18.741 | −30.134 | 10.429 | 1.00 | 39.48 | C |
| ATOM | 7402 | CB | LEU | B | 227 | 19.600 | −29.292 | 11.366 | 1.00 | 33.88 | C |
| ATOM | 7405 | CG | LEU | B | 227 | 19.071 | −27.847 | 11.432 | 1.00 | 35.41 | C |
| ATOM | 7407 | CD1 | LEU | B | 227 | 19.801 | −27.109 | 12.564 | 1.00 | 42.07 | C |
| ATOM | 7411 | CD2 | LEU | B | 227 | 19.371 | −27.165 | 10.051 | 1.00 | 29.07 | C |
| ATOM | 7415 | C | LEU | B | 227 | 19.046 | −31.597 | 10.735 | 1.00 | 36.98 | C |
| ATOM | 7416 | O | LEU | B | 227 | 18.702 | −32.087 | 11.819 | 1.00 | 42.93 | O |
| ATOM | 7417 | N | SER | B | 228 | 19.707 | −32.284 | 9.808 | 1.00 | 37.55 | N |
| ATOM | 7419 | CA | SER | B | 228 | 20.090 | −33.674 | 10.029 | 1.00 | 40.13 | C |
| ATOM | 7421 | CB | SER | B | 228 | 19.364 | −34.576 | 9.029 | 1.00 | 41.00 | C |
| ATOM | 7424 | OG | SER | B | 228 | 17.958 | −34.464 | 9.185 | 1.00 | 41.06 | O |
| ATOM | 7426 | C | SER | B | 228 | 21.607 | −33.851 | 9.898 | 1.00 | 37.71 | C |
| ATOM | 7427 | O | SER | B | 228 | 22.168 | −33.564 | 8.836 | 1.00 | 37.95 | O |
| ATOM | 7428 | N | MET | B | 229 | 22.241 | −34.368 | 10.956 | 1.00 | 37.61 | N |
| ATOM | 7430 | CA | MET | B | 229 | 23.700 | −34.387 | 11.078 | 1.00 | 44.27 | C |
| ATOM | 7432 | CB | MET | B | 229 | 24.131 | −33.388 | 12.156 | 1.00 | 42.75 | C |
| ATOM | 7435 | CG | MET | B | 229 | 24.067 | −31.935 | 11.790 | 1.00 | 55.25 | C |
| ATOM | 7438 | SD | MET | B | 229 | 24.317 | −30.922 | 13.268 | 1.00 | 54.77 | S |
| ATOM | 7439 | CE | MET | B | 229 | 22.880 | −31.129 | 14.129 | 1.00 | 39.93 | C |
| ATOM | 7443 | C | MET | B | 229 | 24.246 | −35.767 | 11.523 | 1.00 | 45.46 | C |
| ATOM | 7444 | O | MET | B | 229 | 23.719 | −36.369 | 12.461 | 1.00 | 33.59 | O |
| ATOM | 7445 | N | SER | B | 230 | 25.330 | −36.216 | 10.890 | 1.00 | 39.48 | N |
| ATOM | 7447 | CA | SER | B | 230 | 26.239 | −37.211 | 11.452 | 1.00 | 42.10 | C |
| ATOM | 7449 | CB | SER | B | 230 | 25.965 | −38.607 | 10.920 | 1.00 | 43.40 | C |
| ATOM | 7452 | OG | SER | B | 230 | 24.601 | −38.940 | 11.052 | 1.00 | 46.87 | O |
| ATOM | 7454 | C | SER | B | 230 | 27.667 | −36.860 | 11.111 | 1.00 | 46.44 | C |
| ATOM | 7455 | O | SER | B | 230 | 27.919 | −35.971 | 10.290 | 1.00 | 40.19 | O |
| ATOM | 7456 | N | ALA | B | 231 | 28.616 | −37.505 | 11.788 | 1.00 | 47.24 | N |
| ATOM | 7458 | CA | ALA | B | 231 | 30.016 | −37.201 | 11.510 | 1.00 | 42.75 | C |
| ATOM | 7460 | CB | ALA | B | 231 | 30.929 | −37.890 | 12.514 | 1.00 | 43.13 | C |
| ATOM | 7464 | C | ALA | B | 231 | 30.304 | −37.701 | 10.104 | 1.00 | 38.80 | C |
| ATOM | 7465 | O | ALA | B | 231 | 29.795 | −38.749 | 9.708 | 1.00 | 49.59 | O |
| ATOM | 7466 | N | ASP | B | 232 | 31.117 | −36.958 | 9.369 | 1.00 | 41.87 | N |
| ATOM | 7468 | CA | ASP | B | 232 | 31.662 | −37.409 | 8.100 | 1.00 | 57.80 | C |
| ATOM | 7470 | CB | ASP | B | 232 | 32.364 | −38.769 | 8.273 | 1.00 | 67.12 | C |
| ATOM | 7473 | CG | ASP | B | 232 | 33.361 | −38.777 | 9.420 | 1.00 | 70.75 | C |
| ATOM | 7474 | OD1 | ASP | B | 232 | 33.509 | −39.840 | 10.069 | 1.00 | 78.73 | O |
| ATOM | 7475 | OD2 | ASP | B | 232 | 34.033 | −37.770 | 9.736 | 1.00 | 69.40 | O |
| ATOM | 7476 | C | ASP | B | 232 | 30.643 | −37.514 | 6.959 | 1.00 | 57.49 | C |
| ATOM | 7477 | O | ASP | B | 232 | 31.010 | −37.957 | 5.868 | 1.00 | 62.82 | O |
| ATOM | 7478 | N | VAL | B | 233 | 29.385 | −37.130 | 7.186 | 1.00 | 47.16 | N |
| ATOM | 7480 | CA | VAL | B | 233 | 28.463 | −36.881 | 6.073 | 1.00 | 45.97 | C |
| ATOM | 7482 | CB | VAL | B | 233 | 27.300 | −37.898 | 6.065 | 1.00 | 49.33 | C |
| ATOM | 7484 | CG1 | VAL | B | 233 | 27.735 | −39.183 | 6.720 | 1.00 | 50.60 | C |
| ATOM | 7488 | CG2 | VAL | B | 233 | 26.080 | −37.338 | 6.749 | 1.00 | 47.06 | C |
| ATOM | 7492 | C | VAL | B | 233 | 27.962 | −35.415 | 5.999 | 1.00 | 43.90 | C |
| ATOM | 7493 | O | VAL | B | 233 | 28.134 | −34.644 | 6.936 | 1.00 | 41.81 | O |
| ATOM | 7494 | N | PRO | B | 234 | 27.484 | −34.999 | 4.832 | 1.00 | 37.51 | N |
| ATOM | 7495 | CA | PRO | B | 234 | 26.777 | −33.723 | 4.680 | 1.00 | 33.79 | C |
| ATOM | 7497 | CB | PRO | B | 234 | 26.260 | −33.792 | 3.241 | 1.00 | 35.43 | C |
| ATOM | 7500 | CG | PRO | B | 234 | 27.217 | −34.753 | 2.527 | 1.00 | 46.17 | C |
| ATOM | 7503 | CD | PRO | B | 234 | 27.598 | −35.742 | 3.558 | 1.00 | 44.23 | C |
| ATOM | 7506 | C | PRO | B | 234 | 25.599 | −33.506 | 5.658 | 1.00 | 34.87 | C |
| ATOM | 7507 | O | PRO | B | 234 | 24.819 | −34.396 | 5.956 | 1.00 | 31.40 | O |
| ATOM | 7508 | N | LEU | B | 235 | 25.548 | −32.306 | 6.208 | 1.00 | 31.32 | N |
| ATOM | 7510 | CA | LEU | B | 235 | 24.331 | −31.741 | 6.802 | 1.00 | 32.76 | C |
| ATOM | 7512 | CB | LEU | B | 235 | 24.612 | −30.290 | 7.231 | 1.00 | 28.84 | C |
| ATOM | 7515 | CG | LEU | B | 235 | 23.492 | −29.413 | 7.794 | 1.00 | 26.12 | C |
| ATOM | 7517 | CD1 | LEU | B | 235 | 22.832 | −30.054 | 9.034 | 1.00 | 29.71 | C |
| ATOM | 7521 | CD2 | LEU | B | 235 | 24.046 | −28.052 | 8.096 | 1.00 | 25.30 | C |
| ATOM | 7525 | C | LEU | B | 235 | 23.246 | −31.678 | 5.767 | 1.00 | 35.07 | C |
| ATOM | 7526 | O | LEU | B | 235 | 23.551 | −31.310 | 4.602 | 1.00 | 27.22 | O |
| ATOM | 7527 | N | VAL | B | 236 | 22.005 | −31.953 | 6.218 | 1.00 | 26.70 | N |
| ATOM | 7529 | CA | VAL | B | 236 | 20.779 | −31.682 | 5.445 | 1.00 | 34.24 | C |
| ATOM | 7531 | CB | VAL | B | 236 | 19.898 | −32.948 | 5.292 | 1.00 | 39.35 | C |
| ATOM | 7533 | CG1 | VAL | B | 236 | 18.752 | −32.702 | 4.395 | 1.00 | 39.25 | C |
| ATOM | 7537 | CG2 | VAL | B | 236 | 20.712 | −34.127 | 4.791 | 1.00 | 58.29 | C |
| ATOM | 7541 | C | VAL | B | 236 | 19.916 | −30.652 | 6.178 | 1.00 | 36.59 | C |
| ATOM | 7542 | O | VAL | B | 236 | 19.483 | −30.871 | 7.311 | 1.00 | 39.43 | O |
| ATOM | 7543 | N | VAL | B | 237 | 19.652 | −29.550 | 5.488 | 1.00 | 33.82 | N |
| ATOM | 7545 | CA | VAL | B | 237 | 18.768 | −28.482 | 5.934 | 1.00 | 29.23 | C |
| ATOM | 7547 | CB | VAL | B | 237 | 19.486 | −27.116 | 5.774 | 1.00 | 29.19 | C |
| ATOM | 7549 | CG1 | VAL | B | 237 | 18.638 | −25.933 | 6.229 | 1.00 | 17.75 | C |
| ATOM | 7553 | CG2 | VAL | B | 237 | 20.829 | −27.114 | 6.514 | 1.00 | 27.29 | C |
| ATOM | 7557 | C | VAL | B | 237 | 17.525 | −28.552 | 5.032 | 1.00 | 27.14 | C |
| ATOM | 7558 | O | VAL | B | 237 | 17.581 | −28.297 | 3.817 | 1.00 | 31.00 | O |
| ATOM | 7559 | N | GLU | B | 238 | 16.382 | −28.844 | 5.650 | 1.00 | 28.29 | N |
| ATOM | 7561 | CA | GLU | B | 238 | 15.145 | −29.104 | 4.912 | 1.00 | 30.47 | C |
| ATOM | 7563 | CB | GLU | B | 238 | 14.623 | −30.515 | 5.208 | 1.00 | 36.24 | C |
| ATOM | 7566 | CG | GLU | B | 238 | 13.330 | −30.946 | 4.488 | 1.00 | 33.25 | C |
| ATOM | 7569 | CD | GLU | B | 238 | 13.150 | −32.467 | 4.467 | 1.00 | 57.69 | C |
| ATOM | 7570 | OE1 | GLU | B | 238 | 13.835 | −33.177 | 5.240 | 1.00 | 56.34 | O |
| ATOM | 7571 | OE2 | GLU | B | 238 | 12.344 | −32.979 | 3.655 | 1.00 | 88.40 | O |
| ATOM | 7572 | C | GLU | B | 238 | 14.072 | −28.117 | 5.305 | 1.00 | 20.17 | C |
| ATOM | 7573 | O | GLU | B | 238 | 13.740 | −28.004 | 6.473 | 1.00 | 26.76 | O |
| ATOM | 7574 | N | TYR | B | 239 | 13.585 | −27.399 | 4.310 | 1.00 | 27.14 | N |
| ATOM | 7576 | CA | TYR | B | 239 | 12.353 | −26.658 | 4.348 | 1.00 | 29.61 | C |
| ATOM | 7578 | CB | TYR | B | 239 | 12.572 | −25.320 | 3.632 | 1.00 | 27.59 | C |
| ATOM | 7581 | CG | TYR | B | 239 | 13.685 | −24.519 | 4.261 | 1.00 | 23.24 | C |
| ATOM | 7582 | CD1 | TYR | B | 239 | 15.015 | −24.693 | 3.855 | 1.00 | 36.14 | C |
| ATOM | 7584 | CE1 | TYR | B | 239 | 16.046 | −24.007 | 4.476 | 1.00 | 38.54 | C |
| ATOM | 7586 | CZ | TYR | B | 239 | 15.760 | −23.157 | 5.545 | 1.00 | 42.94 | C |
| ATOM | 7587 | OH | TYR | B | 239 | 16.763 | −22.419 | 6.153 | 1.00 | 30.48 | O |
| ATOM | 7589 | CE2 | TYR | B | 239 | 14.454 | −23.022 | 5.999 | 1.00 | 26.70 | C |
| ATOM | 7591 | CD2 | TYR | B | 239 | 13.435 | −23.695 | 5.351 | 1.00 | 26.18 | C |
| ATOM | 7593 | C | TYR | B | 239 | 11.283 | −27.496 | 3.645 | 1.00 | 24.38 | C |
| ATOM | 7594 | O | TYR | B | 239 | 11.284 | −27.670 | 2.416 | 1.00 | 27.96 | O |
| ATOM | 7595 | N | LYS | B | 240 | 10.284 | −27.892 | 4.429 | 1.00 | 30.36 | N |
| ATOM | 7597 | CA | LYS | B | 240 | 9.069 | −28.504 | 3.902 | 1.00 | 31.40 | C |
| ATOM | 7599 | CB | LYS | B | 240 | 8.441 | −29.413 | 4.959 | 1.00 | 40.78 | C |
| ATOM | 7602 | CG | LYS | B | 240 | 7.693 | −30.634 | 4.429 | 1.00 | 45.13 | C |
| ATOM | 7605 | CD | LYS | B | 240 | 6.209 | −30.515 | 4.720 | 1.00 | 66.59 | C |
| ATOM | 7608 | CE | LYS | B | 240 | 5.876 | −30.724 | 6.192 | 1.00 | 51.50 | C |
| ATOM | 7611 | NZ | LYS | B | 240 | 4.434 | −30.995 | 6.324 | 1.00 | 47.17 | N |
| ATOM | 7615 | C | LYS | B | 240 | 8.104 | −27.407 | 3.432 | 1.00 | 25.48 | C |
| ATOM | 7616 | O | LYS | B | 240 | 7.784 | −26.472 | 4.157 | 1.00 | 31.57 | O |
| ATOM | 7617 | N | ILE | B | 241 | 7.689 | −27.512 | 2.175 | 1.00 | 24.99 | N |
| ATOM | 7619 | CA | ILE | B | 241 | 6.775 | −26.574 | 1.566 | 1.00 | 34.92 | C |
| ATOM | 7621 | CB | ILE | B | 241 | 7.199 | −26.408 | 0.077 | 1.00 | 18.70 | C |
| ATOM | 7623 | CG1 | ILE | B | 241 | 8.645 | −25.964 | −0.037 | 1.00 | 35.40 | C |
| ATOM | 7626 | CD1 | ILE | B | 241 | 9.093 | −25.796 | −1.466 | 1.00 | 29.29 | C |
| ATOM | 7630 | CG2 | ILE | B | 241 | 6.319 | −25.399 | −0.656 | 1.00 | 46.01 | C |
| ATOM | 7634 | C | ILE | B | 241 | 5.351 | −27.141 | 1.660 | 1.00 | 30.55 | C |
| ATOM | 7635 | O | ILE | B | 241 | 4.897 | −27.723 | 0.689 | 1.00 | 40.63 | O |
| ATOM | 7636 | N | ALA | B | 242 | 4.702 | −27.090 | 2.824 | 1.00 | 37.68 | N |
| ATOM | 7638 | CA | ALA | B | 242 | 3.239 | −27.269 | 2.897 | 1.00 | 37.77 | C |
| ATOM | 7640 | CB | ALA | B | 242 | 2.580 | −26.060 | 2.297 | 1.00 | 34.50 | C |
| ATOM | 7644 | C | ALA | B | 242 | 2.633 | −28.423 | 2.119 | 1.00 | 45.60 | C |
| ATOM | 7645 | O | ALA | B | 242 | 1.976 | −28.137 | 1.122 | 1.00 | 52.96 | O |
| ATOM | 7646 | N | ASP | B | 243 | 2.791 | −29.675 | 2.537 | 1.00 | 48.46 | N |
| ATOM | 7648 | CA | ASP | B | 243 | 2.522 | −30.848 | 1.677 | 1.00 | 48.37 | C |
| ATOM | 7650 | CB | ASP | B | 243 | 1.251 | −31.599 | 2.084 | 1.00 | 59.63 | C |
| ATOM | 7653 | CG | ASP | B | 243 | 1.268 | −33.090 | 1.675 | 1.00 | 63.32 | C |
| ATOM | 7654 | OD1 | ASP | B | 243 | 0.176 | −33.609 | 1.336 | 1.00 | 51.19 | O |
| ATOM | 7655 | OD2 | ASP | B | 243 | 2.274 | −33.847 | 1.755 | 1.00 | 54.34 | O |
| ATOM | 7656 | C | ASP | B | 243 | 2.579 | −30.781 | 0.135 | 1.00 | 55.29 | C |
| ATOM | 7657 | O | ASP | B | 243 | 2.424 | −31.841 | −0.497 | 1.00 | 45.69 | O |
| ATOM | 7658 | N | MET | B | 244 | 2.870 | −29.613 | −0.463 | 1.00 | 49.43 | N |
| ATOM | 7660 | CA | MET | B | 244 | 3.420 | −29.538 | −1.827 | 1.00 | 41.54 | C |
| ATOM | 7662 | CB | MET | B | 244 | 3.510 | −28.081 | −2.260 | 1.00 | 39.41 | C |
| ATOM | 7665 | CG | MET | B | 244 | 3.012 | −27.853 | −3.663 | 1.00 | 49.02 | C |
| ATOM | 7668 | SD | MET | B | 244 | 3.366 | −26.184 | −4.319 | 1.00 | 68.79 | S |
| ATOM | 7669 | CE | MET | B | 244 | 2.342 | −25.299 | −3.317 | 1.00 | 64.60 | C |
| ATOM | 7673 | C | MET | B | 244 | 4.763 | −30.262 | −2.056 | 1.00 | 28.98 | C |
| ATOM | 7674 | O | MET | B | 244 | 4.842 | −31.139 | −2.880 | 1.00 | 31.15 | O |
| ATOM | 7675 | N | GLY | B | 245 | 5.832 | −29.909 | −1.352 | 1.00 | 34.76 | N |
| ATOM | 7677 | CA | GLY | B | 245 | 7.092 | −30.583 | −1.566 | 1.00 | 26.66 | C |
| ATOM | 7680 | C | GLY | B | 245 | 8.099 | −30.211 | −0.539 | 1.00 | 33.02 | C |
| ATOM | 7681 | O | GLY | B | 245 | 7.761 | −30.153 | 0.643 | 1.00 | 33.02 | O |
| ATOM | 7682 | N | HIS | B | 246 | 9.353 | −30.054 | −0.980 | 1.00 | 32.29 | N |
| ATOM | 7684 | CA | HIS | B | 246 | 10.438 | −29.796 | −0.046 | 1.00 | 27.74 | C |
| ATOM | 7686 | CB | HIS | B | 246 | 10.754 | −31.079 | 0.704 | 1.00 | 30.16 | C |
| ATOM | 7689 | CG | HIS | B | 246 | 11.324 | −32.168 | −0.149 | 1.00 | 33.71 | C |
| ATOM | 7690 | ND1 | HIS | B | 246 | 10.757 | −33.421 | −0.216 | 1.00 | 48.88 | N |
| ATOM | 7692 | CE1 | HIS | B | 246 | 11.500 | −34.197 | −0.984 | 1.00 | 55.73 | C |
| ATOM | 7694 | NE2 | HIS | B | 246 | 12.557 | −33.507 | −1.375 | 1.00 | 54.75 | N |
| ATOM | 7696 | CD2 | HIS | B | 246 | 12.477 | −32.238 | −0.857 | 1.00 | 51.80 | C |
| ATOM | 7698 | C | HIS | B | 246 | 11.641 | −29.189 | −0.770 | 1.00 | 31.02 | C |
| ATOM | 7699 | O | HIS | B | 246 | 11.669 | −29.210 | −1.978 | 1.00 | 28.39 | O |
| ATOM | 7700 | N | LEU | B | 247 | 12.480 | −28.441 | −0.063 | 1.00 | 30.36 | N |
| ATOM | 7702 | CA | LEU | B | 247 | 13.727 | −27.949 | −0.604 | 1.00 | 35.36 | C |
| ATOM | 7704 | CB | LEU | B | 247 | 13.612 | −26.439 | −0.776 | 1.00 | 28.89 | C |
| ATOM | 7707 | CG | LEU | B | 247 | 14.749 | −25.580 | −1.314 | 1.00 | 36.12 | C |
| ATOM | 7709 | CD1 | LEU | B | 247 | 15.508 | −26.183 | −2.517 | 1.00 | 39.03 | C |
| ATOM | 7713 | CD2 | LEU | B | 247 | 14.253 | −24.162 | −1.629 | 1.00 | 35.35 | C |
| ATOM | 7717 | C | LEU | B | 247 | 14.783 | −28.361 | 0.430 | 1.00 | 40.12 | C |
| ATOM | 7718 | O | LEU | B | 247 | 14.639 | −28.031 | 1.606 | 1.00 | 46.09 | O |
| ATOM | 7719 | N | LYS | B | 248 | 15.790 | −29.122 | −0.008 | 1.00 | 36.52 | N |
| ATOM | 7721 | CA | LYS | B | 248 | 16.825 | −29.678 | 0.855 | 1.00 | 29.93 | C |
| ATOM | 7723 | CB | LYS | B | 248 | 16.866 | −31.203 | 0.707 | 1.00 | 36.11 | C |
| ATOM | 7726 | CG | LYS | B | 248 | 15.791 | −31.944 | 1.543 | 1.00 | 33.98 | C |
| ATOM | 7729 | CD | LYS | B | 248 | 15.646 | −33.384 | 1.131 | 1.00 | 33.57 | C |
| ATOM | 7732 | CE | LYS | B | 248 | 14.455 | −34.045 | 1.867 | 1.00 | 42.06 | C |
| ATOM | 7735 | NZ | LYS | B | 248 | 14.396 | −35.486 | 1.580 | 1.00 | 39.88 | N |
| ATOM | 7739 | C | LYS | B | 248 | 18.161 | −29.120 | 0.424 | 1.00 | 36.10 | C |
| ATOM | 7740 | O | LYS | B | 248 | 18.455 | −29.077 | −0.774 | 1.00 | 31.89 | O |
| ATOM | 7741 | N | TYR | B | 249 | 18.996 | −28.763 | 1.396 | 1.00 | 33.58 | N |
| ATOM | 7743 | CA | TYR | B | 249 | 20.354 | −28.364 | 1.075 | 1.00 | 39.29 | C |
| ATOM | 7745 | CB | TYR | B | 249 | 20.606 | −26.925 | 1.537 | 1.00 | 31.80 | C |
| ATOM | 7748 | CG | TYR | B | 249 | 19.677 | −25.906 | 0.961 | 1.00 | 32.27 | C |
| ATOM | 7749 | CD1 | TYR | B | 249 | 19.946 | −25.328 | −0.267 | 1.00 | 28.29 | C |
| ATOM | 7751 | CE1 | TYR | B | 249 | 19.172 | −24.306 | −0.752 | 1.00 | 28.17 | C |
| ATOM | 7753 | CZ | TYR | B | 249 | 18.045 | −23.875 | −0.043 | 1.00 | 29.12 | C |
| ATOM | 7754 | OH | TYR | B | 249 | 17.233 | −22.862 | −0.536 | 1.00 | 26.11 | O |
| ATOM | 7756 | CE2 | TYR | B | 249 | 17.721 | −24.472 | 1.139 | 1.00 | 21.27 | C |
| ATOM | 7758 | CD2 | TYR | B | 249 | 18.554 | −25.465 | 1.659 | 1.00 | 35.49 | C |
| ATOM | 7760 | C | TYR | B | 249 | 21.326 | −29.331 | 1.745 | 1.00 | 34.27 | C |
| ATOM | 7761 | O | TYR | B | 249 | 21.109 | −29.728 | 2.880 | 1.00 | 33.08 | O |
| ATOM | 7762 | N | TYR | B | 250 | 22.378 | −29.706 | 1.018 | 1.00 | 32.02 | N |
| ATOM | 7764 | CA | TYR | B | 250 | 23.437 | −30.564 | 1.543 | 1.00 | 29.13 | C |
| ATOM | 7766 | CB | TYR | B | 250 | 23.566 | −31.846 | 0.704 | 1.00 | 35.67 | C |
| ATOM | 7769 | CG | TYR | B | 250 | 22.273 | −32.657 | 0.607 | 1.00 | 32.82 | C |
| ATOM | 7770 | CD1 | TYR | B | 250 | 21.258 | −32.280 | −0.257 | 1.00 | 40.79 | C |
| ATOM | 7772 | CE1 | TYR | B | 250 | 20.107 | −32.987 | −0.350 | 1.00 | 39.49 | C |
| ATOM | 7774 | CZ | TYR | B | 250 | 19.931 | −34.104 | 0.427 | 1.00 | 34.33 | C |
| ATOM | 7775 | OH | TYR | B | 250 | 18.751 | −34.801 | 0.322 | 1.00 | 46.81 | O |
| ATOM | 7777 | CE2 | TYR | B | 250 | 20.914 | −34.517 | 1.281 | 1.00 | 42.30 | C |
| ATOM | 7779 | CD2 | TYR | B | 250 | 22.090 | −33.799 | 1.355 | 1.00 | 36.22 | C |
| ATOM | 7781 | C | TYR | B | 250 | 24.758 | −29.772 | 1.537 | 1.00 | 27.99 | C |
| ATOM | 7782 | O | TYR | B | 250 | 25.148 | −29.130 | 0.529 | 1.00 | 27.95 | O |
| ATOM | 7783 | N | LEU | B | 251 | 25.410 | −29.836 | 2.697 | 1.00 | 30.11 | N |
| ATOM | 7785 | CA | LEU | B | 251 | 26.676 | −29.176 | 2.948 | 1.00 | 30.11 | C |
| ATOM | 7787 | CB | LEU | B | 251 | 26.379 | −27.851 | 3.630 | 1.00 | 26.44 | C |
| ATOM | 7790 | CG | LEU | B | 251 | 27.615 | −27.013 | 3.906 | 1.00 | 28.39 | C |
| ATOM | 7792 | CD1 | LEU | B | 251 | 28.265 | −26.666 | 2.604 | 1.00 | 29.03 | C |
| ATOM | 7796 | CD2 | LEU | B | 251 | 27.332 | −25.825 | 4.768 | 1.00 | 27.20 | C |
| ATOM | 7800 | C | LEU | B | 251 | 27.612 | −30.066 | 3.786 | 1.00 | 33.24 | C |
| ATOM | 7801 | O | LEU | B | 251 | 27.235 | −30.530 | 4.869 | 1.00 | 40.57 | O |
| ATOM | 7802 | N | ALA | B | 252 | 28.784 | −30.384 | 3.238 | 1.00 | 34.95 | N |
| ATOM | 7804 | CA | ALA | B | 252 | 29.800 | −31.220 | 3.926 | 1.00 | 38.80 | C |
| ATOM | 7806 | CB | ALA | B | 252 | 30.929 | −31.709 | 2.894 | 1.00 | 36.54 | C |
| ATOM | 7810 | C | ALA | B | 252 | 30.461 | −30.430 | 5.071 | 1.00 | 34.13 | C |
| ATOM | 7811 | O | ALA | B | 252 | 30.807 | −29.256 | 4.917 | 1.00 | 32.39 | O |
| ATOM | 7812 | N | PRO | B | 253 | 30.634 | −31.072 | 6.216 | 1.00 | 25.58 | N |
| ATOM | 7813 | CA | PRO | B | 253 | 31.342 | −30.463 | 7.338 | 1.00 | 38.91 | C |
| ATOM | 7815 | CB | PRO | B | 253 | 31.184 | −31.484 | 8.468 | 1.00 | 39.67 | C |
| ATOM | 7818 | CG | PRO | B | 253 | 30.855 | −32.776 | 7.802 | 1.00 | 40.87 | C |
| ATOM | 7821 | CD | PRO | B | 253 | 30.105 | −32.404 | 6.565 | 1.00 | 36.33 | C |
| ATOM | 7824 | C | PRO | B | 253 | 32.806 | −30.236 | 7.034 | 1.00 | 40.98 | C |
| ATOM | 7825 | O | PRO | B | 253 | 33.373 | −30.961 | 6.242 | 1.00 | 45.40 | O |
| ATOM | 7826 | N | LYS | B | 254 | 33.364 | −29.159 | 7.559 | 1.00 | 44.07 | N |
| ATOM | 7828 | CA | LYS | B | 254 | 34.800 | −28.996 | 7.566 | 1.00 | 53.05 | C |
| ATOM | 7830 | CB | LYS | B | 254 | 35.145 | −27.681 | 8.270 | 1.00 | 54.14 | C |
| ATOM | 7833 | CG | LYS | B | 254 | 34.331 | −26.474 | 7.786 | 1.00 | 68.53 | C |
| ATOM | 7836 | CD | LYS | B | 254 | 35.166 | −25.476 | 6.978 | 1.00 | 75.88 | C |
| ATOM | 7839 | CE | LYS | B | 254 | 35.810 | −26.140 | 5.752 | 1.00 | 83.68 | C |
| ATOM | 7842 | NZ | LYS | B | 254 | 35.926 | −25.242 | 4.562 | 1.00 | 67.78 | N |
| ATOM | 7846 | C | LYS | B | 254 | 35.362 | −30.220 | 8.301 | 1.00 | 57.69 | C |
| ATOM | 7847 | O | LYS | B | 254 | 34.894 | −30.580 | 9.389 | 1.00 | 62.74 | O |
| ATOM | 7848 | N | ILE | B | 255 | 36.292 | −30.917 | 7.663 | 1.00 | 66.36 | N |
| ATOM | 7850 | CA | ILE | B | 255 | 36.721 | −32.234 | 8.134 | 1.00 | 75.44 | C |
| ATOM | 7852 | CB | ILE | B | 255 | 37.772 | −32.081 | 9.255 | 1.00 | 75.19 | C |
| ATOM | 7854 | CG1 | ILE | B | 255 | 39.075 | −31.530 | 8.683 | 1.00 | 70.42 | C |
| ATOM | 7857 | CD1 | ILE | B | 255 | 39.056 | −30.022 | 8.428 | 1.00 | 58.88 | C |
| ATOM | 7861 | CG2 | ILE | B | 255 | 37.983 | −33.414 | 9.972 | 1.00 | 79.08 | C |
| ATOM | 7865 | C | ILE | B | 255 | 35.537 | −33.060 | 8.654 | 1.00 | 80.93 | C |
| ATOM | 7866 | O | ILE | B | 255 | 34.737 | −33.605 | 7.883 | 1.00 | 85.11 | O |
| ATOM | 7867 | N | MET | C | 1 | 12.734 | 42.003 | −12.242 | 1.00 | 53.76 | N |
| ATOM | 7869 | CA | MET | C | 1 | 12.715 | 40.956 | −11.173 | 1.00 | 38.33 | C |
| ATOM | 7871 | CB | MET | C | 1 | 12.335 | 41.621 | −9.853 | 1.00 | 46.47 | C |
| ATOM | 7874 | CG | MET | C | 1 | 12.954 | 41.011 | −8.589 | 1.00 | 61.59 | C |
| ATOM | 7877 | SD | MET | C | 1 | 11.685 | 40.474 | −7.411 | 1.00 | 69.20 | S |
| ATOM | 7878 | CE | MET | C | 1 | 12.214 | 41.285 | −5.825 | 1.00 | 73.71 | C |
| ATOM | 7882 | C | MET | C | 1 | 11.667 | 39.902 | −11.569 | 1.00 | 47.98 | C |
| ATOM | 7883 | O | MET | C | 1 | 10.532 | 40.259 | −11.903 | 1.00 | 60.74 | O |
| ATOM | 7886 | N | PHE | C | 2 | 12.074 | 38.632 | −11.619 | 1.00 | 42.34 | N |
| ATOM | 7888 | CA | PHE | C | 2 | 11.192 | 37.463 | −11.646 | 1.00 | 41.13 | C |
| ATOM | 7890 | CB | PHE | C | 2 | 11.856 | 36.343 | −12.478 | 1.00 | 45.84 | C |
| ATOM | 7893 | CG | PHE | C | 2 | 11.239 | 34.970 | −12.297 | 1.00 | 45.41 | C |
| ATOM | 7894 | CD1 | PHE | C | 2 | 9.917 | 34.737 | −12.626 | 1.00 | 36.22 | C |
| ATOM | 7896 | CE1 | PHE | C | 2 | 9.362 | 33.469 | −12.468 | 1.00 | 39.20 | C |
| ATOM | 7898 | CZ | PHE | C | 2 | 10.133 | 32.415 | −12.008 | 1.00 | 34.00 | C |
| ATOM | 7900 | CE2 | PHE | C | 2 | 11.452 | 32.630 | −11.700 | 1.00 | 35.25 | C |
| ATOM | 7902 | CD2 | PHE | C | 2 | 12.015 | 33.888 | −11.883 | 1.00 | 43.57 | C |
| ATOM | 7904 | C | PHE | C | 2 | 10.973 | 36.952 | −10.226 | 1.00 | 43.79 | C |
| ATOM | 7905 | O | PHE | C | 2 | 11.947 | 36.805 | −9.471 | 1.00 | 36.22 | O |
| ATOM | 7906 | N | GLU | C | 3 | 9.721 | 36.598 | −9.897 | 1.00 | 38.40 | N |
| ATOM | 7908 | CA | GLU | C | 3 | 9.398 | 36.064 | −8.575 | 1.00 | 31.12 | C |
| ATOM | 7910 | CB | GLU | C | 3 | 9.277 | 37.208 | −7.563 | 1.00 | 28.26 | C |
| ATOM | 7913 | CG | GLU | C | 3 | 9.039 | 36.697 | −6.153 | 1.00 | 34.24 | C |
| ATOM | 7916 | CD | GLU | C | 3 | 8.926 | 37.790 | −5.115 | 1.00 | 41.49 | C |
| ATOM | 7917 | OE1 | GLU | C | 3 | 8.012 | 38.655 | −5.229 | 1.00 | 59.80 | O |
| ATOM | 7918 | OE2 | GLU | C | 3 | 9.721 | 37.727 | −4.150 | 1.00 | 43.59 | O |
| ATOM | 7919 | C | GLU | C | 3 | 8.136 | 35.219 | −8.518 | 1.00 | 28.61 | C |
| ATOM | 7920 | O | GLU | C | 3 | 7.041 | 35.753 | −8.677 | 1.00 | 41.01 | O |
| ATOM | 7921 | N | ALA | C | 4 | 8.278 | 33.946 | −8.149 | 1.00 | 32.18 | N |
| ATOM | 7923 | CA | ALA | C | 4 | 7.234 | 32.940 | −8.295 | 1.00 | 32.44 | C |
| ATOM | 7925 | CB | ALA | C | 4 | 7.535 | 32.030 | −9.466 | 1.00 | 33.70 | C |
| ATOM | 7929 | C | ALA | C | 4 | 7.143 | 32.133 | −7.012 | 1.00 | 34.24 | C |
| ATOM | 7930 | O | ALA | C | 4 | 8.098 | 31.457 | −6.623 | 1.00 | 38.14 | O |
| ATOM | 7931 | N | ARG | C | 5 | 6.007 | 32.241 | −6.331 | 1.00 | 33.24 | N |
| ATOM | 7933 | CA | ARG | C | 5 | 5.860 | 31.623 | −5.020 | 1.00 | 27.21 | C |
| ATOM | 7935 | CB | ARG | C | 5 | 5.340 | 32.627 | −4.002 | 1.00 | 31.11 | C |
| ATOM | 7938 | CG | ARG | C | 5 | 4.852 | 32.058 | −2.678 | 1.00 | 38.11 | C |
| ATOM | 7941 | CD | ARG | C | 5 | 4.145 | 33.106 | −1.813 | 1.00 | 38.74 | C |
| ATOM | 7944 | NE | ARG | C | 5 | 3.696 | 32.576 | −0.521 | 1.00 | 40.45 | N |
| ATOM | 7946 | CZ | ARG | C | 5 | 4.432 | 32.623 | 0.559 | 1.00 | 47.14 | C |
| ATOM | 7947 | NH1 | ARG | C | 5 | 5.637 | 33.186 | 0.511 | 1.00 | 66.40 | N |
| ATOM | 7950 | NH2 | ARG | C | 5 | 3.989 | 32.096 | 1.687 | 1.00 | 32.87 | N |
| ATOM | 7953 | C | ARG | C | 5 | 4.898 | 30.464 | −5.190 | 1.00 | 32.17 | C |
| ATOM | 7954 | O | ARG | C | 5 | 3.732 | 30.669 | −5.562 | 1.00 | 29.82 | O |
| ATOM | 7955 | N | LEU | C | 6 | 5.446 | 29.261 | −5.024 | 1.00 | 22.69 | N |
| ATOM | 7957 | CA | LEU | C | 6 | 4.728 | 28.034 | −5.226 | 1.00 | 21.95 | C |
| ATOM | 7959 | CB | LEU | C | 6 | 5.472 | 27.082 | −6.125 | 1.00 | 33.77 | C |
| ATOM | 7962 | CG | LEU | C | 6 | 4.860 | 25.720 | −6.353 | 1.00 | 33.48 | C |
| ATOM | 7964 | CD1 | LEU | C | 6 | 3.824 | 25.762 | −7.442 | 1.00 | 32.39 | C |
| ATOM | 7968 | CD2 | LEU | C | 6 | 6.019 | 24.848 | −6.783 | 1.00 | 40.20 | C |
| ATOM | 7972 | C | LEU | C | 6 | 4.463 | 27.383 | −3.880 | 1.00 | 28.23 | C |
| ATOM | 7973 | O | LEU | C | 6 | 5.361 | 26.955 | −3.184 | 1.00 | 34.08 | O |
| ATOM | 7974 | N | VAL | C | 7 | 3.195 | 27.266 | −3.552 | 1.00 | 28.65 | N |
| ATOM | 7976 | CA | VAL | C | 7 | 2.747 | 26.743 | −2.274 | 1.00 | 30.75 | C |
| ATOM | 7978 | CB | VAL | C | 7 | 1.237 | 27.062 | −2.140 | 1.00 | 42.13 | C |
| ATOM | 7980 | CG1 | VAL | C | 7 | 0.542 | 26.035 | −1.252 | 1.00 | 53.64 | C |
| ATOM | 7984 | CG2 | VAL | C | 7 | 1.074 | 28.456 | −1.634 | 1.00 | 40.72 | C |
| ATOM | 7988 | C | VAL | C | 7 | 2.935 | 25.234 | −2.096 | 1.00 | 29.81 | C |
| ATOM | 7989 | O | VAL | C | 7 | 3.278 | 24.757 | −1.029 | 1.00 | 30.55 | O |
| ATOM | 7990 | N | GLN | C | 8 | 2.717 | 24.435 | −3.121 | 1.00 | 28.52 | N |
| ATOM | 7992 | CA | GLN | C | 8 | 2.978 | 23.016 | −2.969 | 1.00 | 22.37 | C |
| ATOM | 7994 | CB | GLN | C | 8 | 1.941 | 22.207 | −3.777 | 1.00 | 29.31 | C |
| ATOM | 7997 | CG | GLN | C | 8 | 0.485 | 22.478 | −3.402 | 1.00 | 44.50 | C |
| ATOM | 8000 | CD | GLN | C | 8 | −0.177 | 21.362 | −2.603 | 1.00 | 73.39 | C |
| ATOM | 8001 | OE1 | GLN | C | 8 | −0.170 | 20.203 | −3.029 | 1.00 | 87.78 | O |
| ATOM | 8002 | NE2 | GLN | C | 8 | −0.786 | 21.717 | −1.458 | 1.00 | 72.75 | N |
| ATOM | 8005 | C | GLN | C | 8 | 4.375 | 22.661 | −3.502 | 1.00 | 32.07 | C |
| ATOM | 8006 | O | GLN | C | 8 | 4.547 | 22.390 | −4.687 | 1.00 | 32.05 | O |
| ATOM | 8007 | N | GLY | C | 9 | 5.364 | 22.624 | −2.618 | 1.00 | 35.13 | N |
| ATOM | 8009 | CA | GLY | C | 9 | 6.752 | 22.530 | −3.032 | 1.00 | 32.24 | C |
| ATOM | 8012 | C | GLY | C | 9 | 7.062 | 21.173 | −3.624 | 1.00 | 30.53 | C |
| ATOM | 8013 | O | GLY | C | 9 | 7.911 | 21.065 | −4.501 | 1.00 | 35.63 | O |
| ATOM | 8014 | N | SER | C | 10 | 6.406 | 20.131 | −3.121 | 1.00 | 33.49 | N |
| ATOM | 8016 | CA | SER | C | 10 | 6.529 | 18.790 | −3.680 | 1.00 | 31.68 | C |
| ATOM | 8018 | CB | SER | C | 10 | 5.347 | 17.902 | −3.235 | 1.00 | 20.36 | C |
| ATOM | 8021 | OG | SER | C | 10 | 4.126 | 18.512 | −3.589 | 1.00 | 29.11 | O |
| ATOM | 8023 | C | SER | C | 10 | 6.591 | 18.782 | −5.218 | 1.00 | 27.22 | C |
| ATOM | 8024 | O | SER | C | 10 | 7.241 | 17.931 | −5.795 | 1.00 | 23.33 | O |
| ATOM | 8025 | N | ILE | C | 11 | 5.789 | 19.628 | −5.856 | 1.00 | 23.42 | N |
| ATOM | 8027 | CA | ILE | C | 11 | 5.620 | 19.628 | −7.301 | 1.00 | 32.07 | C |
| ATOM | 8029 | CB | ILE | C | 11 | 4.606 | 20.719 | −7.699 | 1.00 | 23.67 | C |
| ATOM | 8031 | CG1 | ILE | C | 11 | 3.170 | 20.213 | −7.500 | 1.00 | 41.29 | C |
| ATOM | 8034 | CD1 | ILE | C | 11 | 2.078 | 21.330 | −7.797 | 1.00 | 40.66 | C |
| ATOM | 8038 | CG2 | ILE | C | 11 | 4.753 | 21.122 | −9.216 | 1.00 | 30.56 | C |
| ATOM | 8042 | C | ILE | C | 11 | 6.961 | 19.884 | −8.007 | 1.00 | 19.20 | C |
| ATOM | 8043 | O | ILE | C | 11 | 7.303 | 19.248 | −9.005 | 1.00 | 26.05 | O |
| ATOM | 8044 | N | LEU | C | 12 | 7.681 | 20.866 | −7.525 | 1.00 | 21.04 | N |
| ATOM | 8046 | CA | LEU | C | 12 | 8.952 | 21.232 | −8.157 | 1.00 | 27.54 | C |
| ATOM | 8048 | CB | LEU | C | 12 | 9.390 | 22.621 | −7.693 | 1.00 | 26.29 | C |
| ATOM | 8051 | CG | LEU | C | 12 | 10.568 | 23.247 | −8.466 | 1.00 | 31.35 | C |
| ATOM | 8053 | CD1 | LEU | C | 12 | 10.252 | 23.529 | −9.924 | 1.00 | 33.87 | C |
| ATOM | 8057 | CD2 | LEU | C | 12 | 10.921 | 24.562 | −7.840 | 1.00 | 23.19 | C |
| ATOM | 8061 | C | LEU | C | 12 | 10.010 | 20.199 | −7.820 | 1.00 | 22.46 | C |
| ATOM | 8062 | O | LEU | C | 12 | 10.903 | 19.910 | −8.632 | 1.00 | 25.60 | O |
| ATOM | 8063 | N | LYS | C | 13 | 9.941 | 19.648 | −6.605 | 1.00 | 21.21 | N |
| ATOM | 8065 | CA | LYS | C | 13 | 10.797 | 18.498 | −6.263 | 1.00 | 23.29 | C |
| ATOM | 8067 | CB | LYS | C | 13 | 10.506 | 17.977 | −4.838 | 1.00 | 26.14 | C |
| ATOM | 8070 | CG | LYS | C | 13 | 11.057 | 18.999 | −3.772 | 1.00 | 32.63 | C |
| ATOM | 8073 | CD | LYS | C | 13 | 10.510 | 18.711 | −2.372 | 1.00 | 25.36 | C |
| ATOM | 8076 | CE | LYS | C | 13 | 10.992 | 17.357 | −1.801 | 1.00 | 28.04 | C |
| ATOM | 8079 | NZ | LYS | C | 13 | 12.440 | 17.326 | −1.544 | 1.00 | 23.43 | N |
| ATOM | 8083 | C | LYS | C | 13 | 10.669 | 17.383 | −7.260 | 1.00 | 29.86 | C |
| ATOM | 8084 | O | LYS | C | 13 | 11.686 | 16.860 | −7.751 | 1.00 | 29.77 | O |
| ATOM | 8085 | N | LYS | C | 14 | 9.421 | 16.981 | −7.503 | 1.00 | 24.65 | N |
| ATOM | 8087 | CA | LYS | C | 14 | 9.122 | 15.864 | −8.397 | 1.00 | 30.22 | C |
| ATOM | 8089 | CB | LYS | C | 14 | 7.615 | 15.569 | −8.328 | 1.00 | 24.55 | C |
| ATOM | 8092 | CG | LYS | C | 14 | 7.220 | 14.955 | −6.984 | 1.00 | 32.17 | C |
| ATOM | 8095 | CD | LYS | C | 14 | 5.690 | 14.922 | −6.826 | 1.00 | 26.04 | C |
| ATOM | 8098 | CE | LYS | C | 14 | 5.258 | 14.307 | −5.455 | 1.00 | 28.82 | C |
| ATOM | 8101 | NZ | LYS | C | 14 | 3.787 | 14.150 | −5.514 | 1.00 | 40.33 | N |
| ATOM | 8105 | C | LYS | C | 14 | 9.502 | 16.110 | −9.863 | 1.00 | 20.61 | C |
| ATOM | 8106 | O | LYS | C | 14 | 9.726 | 15.175 | −10.588 | 1.00 | 24.71 | O |
| ATOM | 8107 | N | VAL | C | 15 | 9.253 | 17.314 | −10.349 | 1.00 | 24.80 | N |
| ATOM | 8109 | CA | VAL | C | 15 | 9.633 | 17.694 | −11.705 | 1.00 | 28.32 | C |
| ATOM | 8111 | CB | VAL | C | 15 | 9.287 | 19.143 | −11.995 | 1.00 | 31.37 | C |
| ATOM | 8113 | CG1 | VAL | C | 15 | 10.000 | 19.589 | −13.263 | 1.00 | 41.03 | C |
| ATOM | 8117 | CG2 | VAL | C | 15 | 7.779 | 19.257 | −12.165 | 1.00 | 34.69 | C |
| ATOM | 8121 | C | VAL | C | 15 | 11.128 | 17.525 | −11.916 | 1.00 | 21.68 | C |
| ATOM | 8122 | O | VAL | C | 15 | 11.541 | 16.915 | −12.887 | 1.00 | 32.00 | O |
| ATOM | 8123 | N | LEU | C | 16 | 11.927 | 18.019 | −10.972 | 1.00 | 29.28 | N |
| ATOM | 8125 | CA | LEU | C | 16 | 13.372 | 17.871 | −11.076 | 1.00 | 24.93 | C |
| ATOM | 8127 | CB | LEU | C | 16 | 14.095 | 18.684 | −9.997 | 1.00 | 15.38 | C |
| ATOM | 8130 | CG | LEU | C | 16 | 14.433 | 20.114 | −10.462 | 1.00 | 21.35 | C |
| ATOM | 8132 | CD1 | LEU | C | 16 | 13.241 | 20.880 | −11.003 | 1.00 | 29.85 | C |
| ATOM | 8136 | CD2 | LEU | C | 16 | 15.554 | 20.014 | −11.564 | 1.00 | 38.46 | C |
| ATOM | 8140 | C | LEU | C | 16 | 13.741 | 16.394 | −11.005 | 1.00 | 34.17 | C |
| ATOM | 8141 | O | LEU | C | 16 | 14.611 | 15.943 | −11.733 | 1.00 | 38.44 | O |
| ATOM | 8142 | N | GLU | C | 17 | 13.090 | 15.632 | −10.127 | 1.00 | 32.88 | N |
| ATOM | 8144 | CA | GLU | C | 17 | 13.414 | 14.218 | −10.004 | 1.00 | 29.90 | C |
| ATOM | 8146 | CB | GLU | C | 17 | 12.698 | 13.529 | −8.825 | 1.00 | 35.14 | C |
| ATOM | 8149 | CG | GLU | C | 17 | 13.326 | 13.756 | −7.459 | 1.00 | 45.21 | C |
| ATOM | 8152 | CD | GLU | C | 17 | 14.635 | 13.013 | −7.232 | 1.00 | 58.20 | C |
| ATOM | 8153 | OE1 | GLU | C | 17 | 15.717 | 13.590 | −7.494 | 1.00 | 59.34 | O |
| ATOM | 8154 | OE2 | GLU | C | 17 | 14.600 | 11.879 | −6.708 | 1.00 | 71.77 | O |
| ATOM | 8155 | C | GLU | C | 17 | 13.071 | 13.487 | −11.288 | 1.00 | 31.89 | C |
| ATOM | 8156 | O | GLU | C | 17 | 13.676 | 12.467 | −11.585 | 1.00 | 38.30 | O |
| ATOM | 8157 | N | ALA | C | 18 | 12.109 | 14.006 | −12.045 | 1.00 | 30.83 | N |
| ATOM | 8159 | CA | ALA | C | 18 | 11.686 | 13.386 | −13.300 | 1.00 | 34.38 | C |
| ATOM | 8161 | CB | ALA | C | 18 | 10.205 | 13.658 | −13.544 | 1.00 | 27.31 | C |
| ATOM | 8165 | C | ALA | C | 18 | 12.479 | 13.803 | −14.542 | 1.00 | 36.70 | C |
| ATOM | 8166 | O | ALA | C | 18 | 12.227 | 13.250 | −15.624 | 1.00 | 48.97 | O |
| ATOM | 8167 | N | LEU | C | 19 | 13.388 | 14.776 | −14.405 | 1.00 | 28.34 | N |
| ATOM | 8169 | CA | LEU | C | 19 | 14.111 | 15.341 | −15.538 | 1.00 | 28.91 | C |
| ATOM | 8171 | CB | LEU | C | 19 | 13.816 | 16.836 | −15.623 | 1.00 | 23.87 | C |
| ATOM | 8174 | CG | LEU | C | 19 | 12.490 | 17.278 | −16.267 | 1.00 | 37.39 | C |
| ATOM | 8176 | CD1 | LEU | C | 19 | 12.305 | 18.772 | −16.045 | 1.00 | 37.69 | C |
| ATOM | 8180 | CD2 | LEU | C | 19 | 12.503 | 17.038 | −17.734 | 1.00 | 41.19 | C |
| ATOM | 8184 | C | LEU | C | 19 | 15.650 | 15.167 | −15.463 | 1.00 | 34.54 | C |
| ATOM | 8185 | O | LEU | C | 19 | 16.337 | 15.178 | −16.480 | 1.00 | 30.64 | O |
| ATOM | 8186 | N | LYS | C | 20 | 16.178 | 15.044 | −14.253 | 1.00 | 30.01 | N |
| ATOM | 8188 | CA | LYS | C | 20 | 17.579 | 15.185 | −13.979 | 1.00 | 35.49 | C |
| ATOM | 8190 | CB | LYS | C | 20 | 17.811 | 15.324 | −12.472 | 1.00 | 34.51 | C |
| ATOM | 8193 | CG | LYS | C | 20 | 17.528 | 14.052 | −11.655 | 1.00 | 46.74 | C |
| ATOM | 8196 | CD | LYS | C | 20 | 18.161 | 14.121 | −10.266 | 1.00 | 51.15 | C |
| ATOM | 8199 | CE | LYS | C | 20 | 18.159 | 12.773 | −9.564 | 1.00 | 50.19 | C |
| ATOM | 8202 | NZ | LYS | C | 20 | 18.103 | 12.885 | −8.069 | 1.00 | 71.89 | N |
| ATOM | 8206 | C | LYS | C | 20 | 18.349 | 13.997 | −14.564 | 1.00 | 39.68 | C |
| ATOM | 8207 | O | LYS | C | 20 | 19.498 | 14.163 | −14.890 | 1.00 | 35.03 | O |
| ATOM | 8208 | N | ASP | C | 21 | 17.748 | 12.813 | −14.686 | 1.00 | 39.57 | N |
| ATOM | 8210 | CA | ASP | C | 21 | 18.475 | 11.656 | −15.207 | 1.00 | 40.58 | C |
| ATOM | 8212 | CB | ASP | C | 21 | 17.832 | 10.336 | −14.764 | 1.00 | 40.24 | C |
| ATOM | 8215 | CG | ASP | C | 21 | 18.079 | 10.022 | −13.291 | 1.00 | 49.73 | C |
| ATOM | 8216 | OD1 | ASP | C | 21 | 19.205 | 10.245 | −12.787 | 1.00 | 48.80 | O |
| ATOM | 8217 | OD2 | ASP | C | 21 | 17.193 | 9.543 | −12.553 | 1.00 | 71.94 | O |
| ATOM | 8218 | C | ASP | C | 21 | 18.502 | 11.690 | −16.724 | 1.00 | 45.56 | C |
| ATOM | 8219 | O | ASP | C | 21 | 19.412 | 11.150 | −17.345 | 1.00 | 54.75 | O |
| ATOM | 8220 | N | LEU | C | 22 | 17.516 | 12.347 | −17.316 | 1.00 | 41.38 | N |
| ATOM | 8222 | CA | LEU | C | 22 | 17.284 | 12.250 | −18.744 | 1.00 | 40.20 | C |
| ATOM | 8224 | CB | LEU | C | 22 | 15.788 | 12.434 | −19.030 | 1.00 | 41.82 | C |
| ATOM | 8227 | CG | LEU | C | 22 | 15.241 | 12.249 | −20.449 | 1.00 | 49.61 | C |
| ATOM | 8229 | CD1 | LEU | C | 22 | 15.655 | 10.888 | −21.079 | 1.00 | 43.90 | C |
| ATOM | 8233 | CD2 | LEU | C | 22 | 13.708 | 12.393 | −20.428 | 1.00 | 51.71 | C |
| ATOM | 8237 | C | LEU | C | 22 | 18.048 | 13.375 | −19.386 | 1.00 | 40.45 | C |
| ATOM | 8238 | O | LEU | C | 22 | 18.527 | 13.255 | −20.487 | 1.00 | 40.28 | O |
| ATOM | 8239 | N | ILE | C | 23 | 18.119 | 14.507 | −18.702 | 1.00 | 42.69 | N |
| ATOM | 8241 | CA | ILE | C | 23 | 18.792 | 15.657 | −19.243 | 1.00 | 40.35 | C |
| ATOM | 8243 | CB | ILE | C | 23 | 17.815 | 16.607 | −19.957 | 1.00 | 44.54 | C |
| ATOM | 8245 | CG1 | ILE | C | 23 | 17.771 | 17.981 | −19.323 | 1.00 | 49.12 | C |
| ATOM | 8248 | CD1 | ILE | C | 23 | 16.447 | 18.659 | −19.648 | 1.00 | 62.57 | C |
| ATOM | 8252 | CG2 | ILE | C | 23 | 16.409 | 16.058 | −20.054 | 1.00 | 57.32 | C |
| ATOM | 8256 | C | ILE | C | 23 | 19.639 | 16.377 | −18.191 | 1.00 | 44.75 | C |
| ATOM | 8257 | O | ILE | C | 23 | 19.262 | 16.459 | −17.034 | 1.00 | 46.41 | O |
| ATOM | 8258 | N | ASN | C | 24 | 20.834 | 16.828 | −18.560 | 1.00 | 47.33 | N |
| ATOM | 8260 | CA | ASN | C | 24 | 21.731 | 17.446 | −17.574 | 1.00 | 52.01 | C |
| ATOM | 8262 | CB | ASN | C | 24 | 23.203 | 17.158 | −17.856 | 1.00 | 46.37 | C |
| ATOM | 8265 | CG | ASN | C | 24 | 23.704 | 16.001 | −17.073 | 1.00 | 65.86 | C |
| ATOM | 8266 | OD1 | ASN | C | 24 | 23.214 | 14.883 | −17.241 | 1.00 | 71.35 | O |
| ATOM | 8267 | ND2 | ASN | C | 24 | 24.654 | 16.256 | −16.166 | 1.00 | 73.23 | N |
| ATOM | 8270 | C | ASN | C | 24 | 21.596 | 18.943 | −17.520 | 1.00 | 48.30 | C |
| ATOM | 8271 | O | ASN | C | 24 | 21.740 | 19.538 | −16.453 | 1.00 | 49.72 | O |
| ATOM | 8272 | N | GLU | C | 25 | 21.409 | 19.548 | −18.687 | 1.00 | 42.87 | N |
| ATOM | 8274 | CA | GLU | C | 25 | 21.334 | 20.989 | −18.784 | 1.00 | 42.13 | C |
| ATOM | 8276 | CB | GLU | C | 25 | 22.631 | 21.543 | −19.326 | 1.00 | 43.93 | C |
| ATOM | 8279 | CG | GLU | C | 25 | 23.815 | 21.345 | −18.424 | 1.00 | 61.68 | C |
| ATOM | 8282 | CD | GLU | C | 25 | 25.099 | 21.479 | −19.207 | 1.00 | 86.36 | C |
| ATOM | 8283 | OE1 | GLU | C | 25 | 25.011 | 21.612 | −20.459 | 1.00 | 81.98 | O |
| ATOM | 8284 | OE2 | GLU | C | 25 | 26.171 | 21.449 | −18.562 | 1.00 | 85.34 | O |
| ATOM | 8285 | C | GLU | C | 25 | 20.251 | 21.410 | −19.753 | 1.00 | 37.74 | C |
| ATOM | 8286 | O | GLU | C | 25 | 20.012 | 20.760 | −20.751 | 1.00 | 46.92 | O |
| ATOM | 8287 | N | ALA | C | 26 | 19.676 | 22.577 | −19.493 | 1.00 | 36.41 | N |
| ATOM | 8289 | CA | ALA | C | 26 | 18.573 | 23.073 | −20.277 | 1.00 | 36.84 | C |
| ATOM | 8291 | CB | ALA | C | 26 | 17.361 | 22.140 | −20.119 | 1.00 | 41.72 | C |
| ATOM | 8295 | C | ALA | C | 26 | 18.276 | 24.487 | −19.773 | 1.00 | 41.98 | C |
| ATOM | 8296 | O | ALA | C | 26 | 18.529 | 24.821 | −18.607 | 1.00 | 30.42 | O |
| ATOM | 8297 | N | CYS | C | 27 | 17.790 | 25.318 | −20.690 | 1.00 | 44.26 | N |
| ATOM | 8299 | CA | CYS | C | 27 | 17.396 | 26.701 | −20.432 | 1.00 | 47.90 | C |
| ATOM | 8301 | CB | CYS | C | 27 | 17.571 | 27.510 | −21.730 | 1.00 | 49.15 | C |
| ATOM | 8304 | SG | CYS | C | 27 | 17.751 | 29.287 | −21.541 | 1.00 | 69.93 | S |
| ATOM | 8305 | C | CYS | C | 27 | 15.935 | 26.811 | −20.002 | 1.00 | 51.79 | C |
| ATOM | 8306 | O | CYS | C | 27 | 15.036 | 26.284 | −20.660 | 1.00 | 55.31 | O |
| ATOM | 8307 | N | TRP | C | 28 | 15.704 | 27.573 | −18.942 | 1.00 | 49.40 | N |
| ATOM | 8309 | CA | TRP | C | 28 | 14.372 | 27.787 | −18.441 | 1.00 | 47.94 | C |
| ATOM | 8311 | CB | TRP | C | 28 | 14.352 | 27.760 | −16.901 | 1.00 | 44.81 | C |
| ATOM | 8314 | CG | TRP | C | 28 | 14.678 | 26.400 | −16.333 | 1.00 | 52.57 | C |
| ATOM | 8315 | CD1 | TRP | C | 28 | 15.835 | 25.699 | −16.519 | 1.00 | 60.40 | C |
| ATOM | 8317 | NE1 | TRP | C | 28 | 15.769 | 24.486 | −15.879 | 1.00 | 52.38 | N |
| ATOM | 8319 | CE2 | TRP | C | 28 | 14.564 | 24.386 | −15.231 | 1.00 | 46.82 | C |
| ATOM | 8320 | CD2 | TRP | C | 28 | 13.835 | 25.558 | −15.522 | 1.00 | 48.63 | C |
| ATOM | 8321 | CE3 | TRP | C | 28 | 12.545 | 25.679 | −15.002 | 1.00 | 56.76 | C |
| ATOM | 8323 | CZ3 | TRP | C | 28 | 12.020 | 24.629 | −14.255 | 1.00 | 56.74 | C |
| ATOM | 8325 | CH2 | TRP | C | 28 | 12.760 | 23.466 | −14.007 | 1.00 | 44.57 | C |
| ATOM | 8327 | CZ2 | TRP | C | 28 | 14.032 | 23.330 | −14.481 | 1.00 | 50.97 | C |
| ATOM | 8329 | C | TRP | C | 28 | 13.859 | 29.107 | −19.018 | 1.00 | 46.96 | C |
| ATOM | 8330 | O | TRP | C | 28 | 14.195 | 30.196 | −18.565 | 1.00 | 54.72 | O |
| ATOM | 8331 | N | ASP | C | 29 | 12.952 | 28.977 | −19.978 | 1.00 | 38.32 | N |
| ATOM | 8333 | CA | ASP | C | 29 | 12.289 | 30.092 | −20.587 | 1.00 | 26.63 | C |
| ATOM | 8335 | CB | ASP | C | 29 | 11.726 | 29.650 | −21.949 | 1.00 | 37.93 | C |
| ATOM | 8338 | CG | ASP | C | 29 | 12.815 | 29.119 | −22.903 | 1.00 | 64.85 | C |
| ATOM | 8339 | OD1 | ASP | C | 29 | 13.974 | 29.632 | −22.879 | 1.00 | 66.74 | O |
| ATOM | 8340 | OD2 | ASP | C | 29 | 12.596 | 28.187 | −23.720 | 1.00 | 90.39 | O |
| ATOM | 8341 | C | ASP | C | 29 | 11.184 | 30.606 | −19.675 | 1.00 | 32.84 | C |
| ATOM | 8342 | O | ASP | C | 29 | 10.137 | 29.996 | −19.573 | 1.00 | 38.33 | O |
| ATOM | 8343 | N | ILE | C | 30 | 11.428 | 31.746 | −19.034 | 1.00 | 33.94 | N |
| ATOM | 8345 | CA | ILE | C | 30 | 10.517 | 32.350 | −18.085 | 1.00 | 39.19 | C |
| ATOM | 8347 | CB | ILE | C | 30 | 11.290 | 32.792 | −16.850 | 1.00 | 41.41 | C |
| ATOM | 8349 | CG1 | ILE | C | 30 | 12.153 | 31.661 | −16.304 | 1.00 | 48.36 | C |
| ATOM | 8352 | CD1 | ILE | C | 30 | 11.379 | 30.622 | −15.600 | 1.00 | 37.42 | C |
| ATOM | 8356 | CG2 | ILE | C | 30 | 10.351 | 33.280 | −15.846 | 1.00 | 34.57 | C |
| ATOM | 8360 | C | ILE | C | 30 | 9.846 | 33.595 | −18.644 | 1.00 | 43.75 | C |
| ATOM | 8361 | O | ILE | C | 30 | 10.524 | 34.457 | −19.194 | 1.00 | 39.16 | O |
| ATOM | 8362 | N | SER | C | 31 | 8.532 | 33.713 | −18.425 | 1.00 | 35.62 | N |
| ATOM | 8364 | CA | SER | C | 31 | 7.762 | 34.845 | −18.929 | 1.00 | 31.89 | C |
| ATOM | 8366 | CB | SER | C | 31 | 7.259 | 34.584 | −20.342 | 1.00 | 35.97 | C |
| ATOM | 8369 | OG | SER | C | 31 | 6.005 | 33.931 | −20.354 | 1.00 | 38.69 | O |
| ATOM | 8371 | C | SER | C | 31 | 6.589 | 35.141 | −18.015 | 1.00 | 39.72 | C |
| ATOM | 8372 | O | SER | C | 31 | 6.397 | 34.462 | −16.993 | 1.00 | 33.81 | O |
| ATOM | 8373 | N | SER | C | 32 | 5.834 | 36.186 | −18.358 | 1.00 | 35.65 | N |
| ATOM | 8375 | CA | SER | C | 32 | 4.589 | 36.496 | −17.643 | 1.00 | 37.29 | C |
| ATOM | 8377 | CB | SER | C | 32 | 4.025 | 37.863 | −18.056 | 1.00 | 39.18 | C |
| ATOM | 8380 | OG | SER | C | 32 | 4.015 | 37.960 | −19.471 | 1.00 | 45.45 | O |
| ATOM | 8382 | C | SER | C | 32 | 3.511 | 35.419 | −17.744 | 1.00 | 33.24 | C |
| ATOM | 8383 | O | SER | C | 32 | 2.632 | 35.361 | −16.898 | 1.00 | 33.03 | O |
| ATOM | 8384 | N | SER | C | 33 | 3.551 | 34.571 | −18.765 | 1.00 | 33.80 | N |
| ATOM | 8386 | CA | SER | C | 33 | 2.567 | 33.501 | −18.896 | 1.00 | 35.94 | C |
| ATOM | 8388 | CB | SER | C | 33 | 2.360 | 33.149 | −20.373 | 1.00 | 31.91 | C |
| ATOM | 8391 | OG | SER | C | 33 | 3.594 | 32.817 | −20.960 | 1.00 | 52.98 | O |
| ATOM | 8393 | C | SER | C | 33 | 2.942 | 32.224 | −18.114 | 1.00 | 40.47 | C |
| ATOM | 8394 | O | SER | C | 33 | 2.125 | 31.336 | −17.859 | 1.00 | 40.36 | O |
| ATOM | 8395 | N | GLY | C | 34 | 4.212 | 32.083 | −17.780 | 1.00 | 40.83 | N |
| ATOM | 8397 | CA | GLY | C | 34 | 4.603 | 30.956 | −16.987 | 1.00 | 34.87 | C |
| ATOM | 8400 | C | GLY | C | 34 | 6.041 | 30.573 | −17.217 | 1.00 | 36.39 | C |
| ATOM | 8401 | O | GLY | C | 34 | 6.782 | 31.263 | −17.890 | 1.00 | 31.16 | O |
| ATOM | 8402 | N | VAL | C | 35 | 6.382 | 29.406 | −16.676 | 1.00 | 33.45 | N |
| ATOM | 8404 | CA | VAL | C | 35 | 7.654 | 28.765 | −16.917 | 1.00 | 38.44 | C |
| ATOM | 8406 | CB | VAL | C | 35 | 8.159 | 28.152 | −15.635 | 1.00 | 37.65 | C |
| ATOM | 8408 | CG1 | VAL | C | 35 | 9.561 | 27.583 | −15.823 | 1.00 | 39.22 | C |
| ATOM | 8412 | CG2 | VAL | C | 35 | 8.084 | 29.159 | −14.533 | 1.00 | 27.44 | C |
| ATOM | 8416 | C | VAL | C | 35 | 7.555 | 27.615 | −17.899 | 1.00 | 42.81 | C |
| ATOM | 8417 | O | VAL | C | 35 | 6.714 | 26.723 | −17.767 | 1.00 | 45.89 | O |
| ATOM | 8418 | N | ASN | C | 36 | 8.494 | 27.589 | −18.826 | 1.00 | 41.75 | N |
| ATOM | 8420 | CA | ASN | C | 36 | 8.460 | 26.629 | −19.909 | 1.00 | 53.16 | C |
| ATOM | 8422 | CB | ASN | C | 36 | 7.969 | 27.338 | −21.184 | 1.00 | 53.87 | C |
| ATOM | 8425 | CG | ASN | C | 36 | 7.089 | 28.557 | −20.876 | 1.00 | 82.67 | C |
| ATOM | 8426 | OD1 | ASN | C | 36 | 5.876 | 28.420 | −20.680 | 1.00 | 90.43 | O |
| ATOM | 8427 | ND2 | ASN | C | 36 | 7.698 | 29.752 | −20.822 | 1.00 | 90.82 | N |
| ATOM | 8430 | C | ASN | C | 36 | 9.854 | 26.049 | −20.105 | 1.00 | 45.25 | C |
| ATOM | 8431 | O | ASN | C | 36 | 10.826 | 26.786 | −20.139 | 1.00 | 44.69 | O |
| ATOM | 8432 | N | LEU | C | 37 | 9.977 | 24.736 | −20.233 | 1.00 | 40.60 | N |
| ATOM | 8434 | CA | LEU | C | 37 | 11.276 | 24.187 | −20.611 | 1.00 | 33.05 | C |
| ATOM | 8436 | CB | LEU | C | 37 | 11.913 | 23.472 | −19.428 | 1.00 | 47.23 | C |
| ATOM | 8439 | CG | LEU | C | 37 | 13.263 | 22.762 | −19.586 | 1.00 | 51.62 | C |
| ATOM | 8441 | CD1 | LEU | C | 37 | 13.844 | 22.507 | −18.202 | 1.00 | 52.53 | C |
| ATOM | 8445 | CD2 | LEU | C | 37 | 13.050 | 21.436 | −20.333 | 1.00 | 59.75 | C |
| ATOM | 8449 | C | LEU | C | 37 | 11.090 | 23.280 | −21.820 | 1.00 | 37.34 | C |
| ATOM | 8450 | O | LEU | C | 37 | 10.026 | 22.713 | −22.021 | 1.00 | 38.86 | O |
| ATOM | 8451 | N | GLN | C | 38 | 12.071 | 23.273 | −22.712 | 1.00 | 36.34 | N |
| ATOM | 8453 | CA | GLN | C | 38 | 12.016 | 22.496 | −23.947 | 1.00 | 36.97 | C |
| ATOM | 8455 | CB | GLN | C | 38 | 11.453 | 23.369 | −25.060 | 1.00 | 33.66 | C |
| ATOM | 8458 | CG | GLN | C | 38 | 10.586 | 22.703 | −26.093 | 1.00 | 46.77 | C |
| ATOM | 8461 | CD | GLN | C | 38 | 9.895 | 23.744 | −26.976 | 1.00 | 76.29 | C |
| ATOM | 8462 | OE1 | GLN | C | 38 | 10.566 | 24.450 | −27.739 | 1.00 | 71.13 | O |
| ATOM | 8463 | NE2 | GLN | C | 38 | 8.573 | 23.891 | −26.823 | 1.00 | 58.00 | N |
| ATOM | 8466 | C | GLN | C | 38 | 13.446 | 22.077 | −24.269 | 1.00 | 38.49 | C |
| ATOM | 8467 | O | GLN | C | 38 | 14.303 | 22.929 | −24.440 | 1.00 | 38.17 | O |
| ATOM | 8468 | N | SER | C | 39 | 13.728 | 20.772 | −24.235 | 1.00 | 42.03 | N |
| ATOM | 8470 | CA | SER | C | 39 | 15.088 | 20.287 | −24.486 | 1.00 | 39.38 | C |
| ATOM | 8472 | CB | SER | C | 39 | 15.905 | 20.244 | −23.197 | 1.00 | 41.20 | C |
| ATOM | 8475 | OG | SER | C | 39 | 17.226 | 19.774 | −23.427 | 1.00 | 50.48 | O |
| ATOM | 8477 | C | SER | C | 39 | 15.107 | 18.891 | −25.059 | 1.00 | 40.35 | C |
| ATOM | 8478 | O | SER | C | 39 | 14.351 | 18.013 | −24.654 | 1.00 | 46.01 | O |
| ATOM | 8479 | N | MET | C | 40 | 16.121 | 18.628 | −25.858 | 1.00 | 40.25 | N |
| ATOM | 8481 | CA | MET | C | 40 | 16.536 | 17.257 | −26.056 | 1.00 | 34.14 | C |
| ATOM | 8483 | CB | MET | C | 40 | 17.485 | 17.144 | −27.248 | 1.00 | 45.33 | C |
| ATOM | 8486 | CG | MET | C | 40 | 16.871 | 17.644 | −28.551 | 1.00 | 54.71 | C |
| ATOM | 8489 | SD | MET | C | 40 | 16.556 | 16.292 | −29.692 | 1.00 | 74.41 | S |
| ATOM | 8490 | CE | MET | C | 40 | 14.855 | 16.054 | −29.396 | 1.00 | 64.80 | C |
| ATOM | 8494 | C | MET | C | 40 | 17.222 | 16.695 | −24.845 | 1.00 | 24.49 | C |
| ATOM | 8495 | O | MET | C | 40 | 17.852 | 17.402 | −24.076 | 1.00 | 32.09 | O |
| ATOM | 8496 | N | ASP | C | 41 | 17.130 | 15.376 | −24.710 | 1.00 | 21.44 | N |
| ATOM | 8498 | CA | ASP | C | 41 | 17.917 | 14.669 | −23.729 | 1.00 | 27.58 | C |
| ATOM | 8500 | CB | ASP | C | 41 | 17.330 | 13.284 | −23.442 | 1.00 | 19.79 | C |
| ATOM | 8503 | CG | ASP | C | 41 | 17.598 | 12.277 | −24.539 | 1.00 | 39.81 | C |
| ATOM | 8504 | OD1 | ASP | C | 41 | 18.645 | 12.339 | −25.207 | 1.00 | 43.61 | O |
| ATOM | 8505 | OD2 | ASP | C | 41 | 16.810 | 11.346 | −24.783 | 1.00 | 45.20 | O |
| ATOM | 8506 | C | ASP | C | 41 | 19.401 | 14.602 | −24.142 | 1.00 | 37.58 | C |
| ATOM | 8507 | O | ASP | C | 41 | 19.778 | 15.104 | −25.222 | 1.00 | 36.43 | O |
| ATOM | 8508 | N | SER | C | 42 | 20.224 | 14.097 | −23.215 | 1.00 | 39.33 | N |
| ATOM | 8510 | CA | SER | C | 42 | 21.686 | 14.195 | −23.294 | 1.00 | 48.14 | C |
| ATOM | 8512 | CB | SER | C | 42 | 22.311 | 13.958 | −21.906 | 1.00 | 54.36 | C |
| ATOM | 8515 | OG | SER | C | 42 | 21.827 | 12.767 | −21.288 | 1.00 | 45.63 | O |
| ATOM | 8517 | C | SER | C | 42 | 22.297 | 13.229 | −24.331 | 1.00 | 48.17 | C |
| ATOM | 8518 | O | SER | C | 42 | 23.433 | 13.398 | −24.758 | 1.00 | 51.35 | O |
| ATOM | 8519 | N | SER | C | 43 | 21.524 | 12.239 | −24.757 | 1.00 | 47.10 | N |
| ATOM | 8521 | CA | SER | C | 43 | 21.939 | 11.318 | −25.819 | 1.00 | 48.11 | C |
| ATOM | 8523 | CB | SER | C | 43 | 21.546 | 9.873 | −25.486 | 1.00 | 53.89 | C |
| ATOM | 8526 | OG | SER | C | 43 | 21.770 | 9.545 | −24.122 | 1.00 | 55.80 | O |
| ATOM | 8528 | C | SER | C | 43 | 21.322 | 11.634 | −27.170 | 1.00 | 43.57 | C |
| ATOM | 8529 | O | SER | C | 43 | 21.370 | 10.774 | −28.037 | 1.00 | 48.41 | O |
| ATOM | 8530 | N | HIS | C | 44 | 20.685 | 12.804 | −27.302 | 1.00 | 35.19 | N |
| ATOM | 8532 | CA | HIS | C | 44 | 20.067 | 13.279 | −28.540 | 1.00 | 42.96 | C |
| ATOM | 8534 | CB | HIS | C | 44 | 21.143 | 13.713 | −29.553 | 1.00 | 46.70 | C |
| ATOM | 8537 | CG | HIS | C | 44 | 21.931 | 14.911 | −29.111 | 1.00 | 62.15 | C |
| ATOM | 8538 | ND1 | HIS | C | 44 | 23.072 | 14.813 | −28.336 | 1.00 | 52.61 | N |
| ATOM | 8540 | CE1 | HIS | C | 44 | 23.521 | 16.026 | −28.066 | 1.00 | 73.23 | C |
| ATOM | 8542 | NE2 | HIS | C | 44 | 22.711 | 16.908 | −28.629 | 1.00 | 71.52 | N |
| ATOM | 8544 | CD2 | HIS | C | 44 | 21.697 | 16.237 | −29.270 | 1.00 | 66.18 | C |
| ATOM | 8546 | C | HIS | C | 44 | 19.016 | 12.326 | −29.160 | 1.00 | 46.81 | C |
| ATOM | 8547 | O | HIS | C | 44 | 18.897 | 12.200 | −30.383 | 1.00 | 52.12 | O |
| ATOM | 8548 | N | VAL | C | 45 | 18.209 | 11.702 | −28.307 | 1.00 | 41.04 | N |
| ATOM | 8550 | CA | VAL | C | 45 | 17.119 | 10.823 | −28.747 | 1.00 | 31.71 | C |
| ATOM | 8552 | CB | VAL | C | 45 | 17.270 | 9.442 | −28.104 | 1.00 | 38.21 | C |
| ATOM | 8554 | CG1 | VAL | C | 45 | 16.138 | 8.494 | −28.517 | 1.00 | 26.84 | C |
| ATOM | 8558 | CG2 | VAL | C | 45 | 18.661 | 8.854 | −28.468 | 1.00 | 36.22 | C |
| ATOM | 8562 | C | VAL | C | 45 | 15.733 | 11.398 | −28.451 | 1.00 | 34.51 | C |
| ATOM | 8563 | O | VAL | C | 45 | 14.942 | 11.541 | −29.364 | 1.00 | 39.03 | O |
| ATOM | 8564 | N | SER | C | 46 | 15.405 | 11.706 | −27.189 | 1.00 | 36.49 | N |
| ATOM | 8566 | CA | SER | C | 46 | 14.035 | 12.147 | −26.892 | 1.00 | 30.18 | C |
| ATOM | 8568 | CB | SER | C | 46 | 13.469 | 11.452 | −25.652 | 1.00 | 27.98 | C |
| ATOM | 8571 | OG | SER | C | 46 | 14.164 | 11.863 | −24.462 | 1.00 | 30.24 | O |
| ATOM | 8573 | C | SER | C | 46 | 13.989 | 13.642 | −26.669 | 1.00 | 34.06 | C |
| ATOM | 8574 | O | SER | C | 46 | 15.015 | 14.335 | −26.562 | 1.00 | 34.09 | O |
| ATOM | 8575 | N | LEU | C | 47 | 12.758 | 14.125 | −26.635 | 1.00 | 32.39 | N |
| ATOM | 8577 | CA | LEU | C | 47 | 12.461 | 15.527 | −26.441 | 1.00 | 35.52 | C |
| ATOM | 8579 | CB | LEU | C | 47 | 11.762 | 16.048 | −27.699 | 1.00 | 36.66 | C |
| ATOM | 8582 | CG | LEU | C | 47 | 11.395 | 17.521 | −27.841 | 1.00 | 54.57 | C |
| ATOM | 8584 | CD1 | LEU | C | 47 | 12.621 | 18.415 | −27.857 | 1.00 | 46.97 | C |
| ATOM | 8588 | CD2 | LEU | C | 47 | 10.589 | 17.718 | −29.113 | 1.00 | 65.68 | C |
| ATOM | 8592 | C | LEU | C | 47 | 11.550 | 15.642 | −25.207 | 1.00 | 35.92 | C |
| ATOM | 8593 | O | LEU | C | 47 | 10.828 | 14.706 | −24.870 | 1.00 | 28.74 | O |
| ATOM | 8594 | N | VAL | C | 48 | 11.661 | 16.763 | −24.504 | 1.00 | 28.65 | N |
| ATOM | 8596 | CA | VAL | C | 48 | 10.959 | 16.988 | −23.264 | 1.00 | 33.89 | C |
| ATOM | 8598 | CB | VAL | C | 48 | 11.938 | 17.061 | −22.053 | 1.00 | 38.04 | C |
| ATOM | 8600 | CG1 | VAL | C | 48 | 11.181 | 17.423 | −20.819 | 1.00 | 38.69 | C |
| ATOM | 8604 | CG2 | VAL | C | 48 | 12.608 | 15.771 | −21.809 | 1.00 | 25.05 | C |
| ATOM | 8608 | C | VAL | C | 48 | 10.347 | 18.370 | −23.446 | 1.00 | 32.12 | C |
| ATOM | 8609 | O | VAL | C | 48 | 10.973 | 19.284 | −23.995 | 1.00 | 31.45 | O |
| ATOM | 8610 | N | GLN | C | 49 | 9.120 | 18.515 | −22.976 | 1.00 | 23.34 | N |
| ATOM | 8612 | CA | GLN | C | 49 | 8.506 | 19.820 | −22.866 | 1.00 | 22.72 | C |
| ATOM | 8614 | CB | GLN | C | 49 | 7.642 | 20.062 | −24.079 | 1.00 | 26.36 | C |
| ATOM | 8617 | CG | GLN | C | 49 | 7.123 | 21.462 | −24.184 | 1.00 | 46.51 | C |
| ATOM | 8620 | CD | GLN | C | 49 | 5.743 | 21.469 | −24.770 | 1.00 | 60.24 | C |
| ATOM | 8621 | OE1 | GLN | C | 49 | 5.593 | 21.470 | −25.993 | 1.00 | 71.63 | O |
| ATOM | 8622 | NE2 | GLN | C | 49 | 4.727 | 21.411 | −23.906 | 1.00 | 54.16 | N |
| ATOM | 8625 | C | GLN | C | 49 | 7.753 | 19.925 | −21.552 | 1.00 | 29.00 | C |
| ATOM | 8626 | O | GLN | C | 49 | 6.839 | 19.130 | −21.248 | 1.00 | 26.56 | O |
| ATOM | 8627 | N | LEU | C | 50 | 8.214 | 20.876 | −20.748 | 1.00 | 33.31 | N |
| ATOM | 8629 | CA | LEU | C | 50 | 7.631 | 21.165 | −19.457 | 1.00 | 30.33 | C |
| ATOM | 8631 | CB | LEU | C | 50 | 8.705 | 21.212 | −18.369 | 1.00 | 27.95 | C |
| ATOM | 8634 | CG | LEU | C | 50 | 8.194 | 21.650 | −16.988 | 1.00 | 32.83 | C |
| ATOM | 8636 | CD1 | LEU | C | 50 | 7.535 | 20.456 | −16.312 | 1.00 | 38.72 | C |
| ATOM | 8640 | CD2 | LEU | C | 50 | 9.338 | 22.226 | −16.125 | 1.00 | 36.69 | C |
| ATOM | 8644 | C | LEU | C | 50 | 6.883 | 22.492 | −19.494 | 1.00 | 33.48 | C |
| ATOM | 8645 | O | LEU | C | 50 | 7.402 | 23.470 | −20.035 | 1.00 | 34.36 | O |
| ATOM | 8646 | N | THR | C | 51 | 5.697 | 22.499 | −18.865 | 1.00 | 30.41 | N |
| ATOM | 8648 | CA | THR | C | 51 | 4.928 | 23.710 | −18.636 | 1.00 | 30.95 | C |
| ATOM | 8650 | CB | THR | C | 51 | 3.854 | 23.873 | −19.717 | 1.00 | 42.12 | C |
| ATOM | 8652 | OG1 | THR | C | 51 | 4.387 | 23.582 | −21.013 | 1.00 | 38.77 | O |
| ATOM | 8654 | CG2 | THR | C | 51 | 3.443 | 25.299 | −19.810 | 1.00 | 40.52 | C |
| ATOM | 8658 | C | THR | C | 51 | 4.283 | 23.860 | −17.248 | 1.00 | 27.21 | C |
| ATOM | 8659 | O | THR | C | 51 | 3.495 | 23.036 | −16.763 | 1.00 | 28.68 | O |
| ATOM | 8660 | N | LEU | C | 52 | 4.548 | 25.012 | −16.675 | 1.00 | 25.43 | N |
| ATOM | 8662 | CA | LEU | C | 52 | 4.025 | 25.417 | −15.388 | 1.00 | 25.74 | C |
| ATOM | 8664 | CB | LEU | C | 52 | 5.187 | 25.534 | −14.399 | 1.00 | 31.39 | C |
| ATOM | 8667 | CG | LEU | C | 52 | 6.017 | 24.292 | −14.133 | 1.00 | 31.48 | C |
| ATOM | 8669 | CD1 | LEU | C | 52 | 7.144 | 24.587 | −13.179 | 1.00 | 31.97 | C |
| ATOM | 8673 | CD2 | LEU | C | 52 | 5.107 | 23.166 | −13.597 | 1.00 | 41.60 | C |
| ATOM | 8677 | C | LEU | C | 52 | 3.464 | 26.814 | −15.611 | 1.00 | 22.31 | C |
| ATOM | 8678 | O | LEU | C | 52 | 4.231 | 27.757 | −15.660 | 1.00 | 28.87 | O |
| ATOM | 8679 | N | ARG | C | 53 | 2.143 | 26.935 | −15.664 | 1.00 | 29.84 | N |
| ATOM | 8681 | CA | ARG | C | 53 | 1.447 | 28.200 | −15.928 | 1.00 | 31.23 | C |
| ATOM | 8683 | CB | ARG | C | 53 | −0.041 | 27.955 | −16.274 | 1.00 | 31.01 | C |
| ATOM | 8686 | CG | ARG | C | 53 | −0.305 | 27.218 | −17.594 | 1.00 | 39.99 | C |
| ATOM | 8689 | CD | ARG | C | 53 | −1.835 | 26.940 | −17.851 | 1.00 | 50.33 | C |
| ATOM | 8692 | NE | ARG | C | 53 | −2.104 | 25.517 | −18.106 | 1.00 | 64.93 | N |
| ATOM | 8694 | CZ | ARG | C | 53 | −2.780 | 24.710 | −17.287 | 1.00 | 76.99 | C |
| ATOM | 8695 | NH1 | ARG | C | 53 | −3.267 | 25.175 | −16.142 | 1.00 | 82.17 | N |
| ATOM | 8698 | NH2 | ARG | C | 53 | −2.950 | 23.425 | −17.594 | 1.00 | 78.36 | N |
| ATOM | 8701 | C | ARG | C | 53 | 1.497 | 29.126 | −14.731 | 1.00 | 25.10 | C |
| ATOM | 8702 | O | ARG | C | 53 | 1.499 | 28.680 | −13.553 | 1.00 | 24.28 | O |
| ATOM | 8703 | N | SER | C | 54 | 1.571 | 30.422 | −15.038 | 1.00 | 20.85 | N |
| ATOM | 8705 | CA | SER | C | 54 | 1.694 | 31.418 | −14.001 | 1.00 | 25.07 | C |
| ATOM | 8707 | CB | SER | C | 54 | 1.733 | 32.853 | −14.550 | 1.00 | 29.08 | C |
| ATOM | 8710 | OG | SER | C | 54 | 0.713 | 33.103 | −15.493 | 1.00 | 37.23 | O |
| ATOM | 8712 | C | SER | C | 54 | 0.630 | 31.301 | −12.949 | 1.00 | 21.11 | C |
| ATOM | 8713 | O | SER | C | 54 | 0.910 | 31.560 | −11.786 | 1.00 | 25.97 | O |
| ATOM | 8714 | N | GLU | C | 55 | −0.595 | 30.985 | −13.353 | 1.00 | 31.95 | N |
| ATOM | 8716 | CA | GLU | C | 55 | −1.726 | 31.103 | −12.447 | 1.00 | 29.28 | C |
| ATOM | 8718 | CB | GLU | C | 55 | −3.060 | 31.006 | −13.202 | 1.00 | 41.28 | C |
| ATOM | 8721 | CG | GLU | C | 55 | −3.073 | 31.720 | −14.547 | 1.00 | 57.40 | C |
| ATOM | 8724 | CD | GLU | C | 55 | −2.596 | 30.833 | −15.684 | 1.00 | 64.51 | C |
| ATOM | 8725 | OE1 | GLU | C | 55 | −3.151 | 29.726 | −15.892 | 1.00 | 71.46 | O |
| ATOM | 8726 | OE2 | GLU | C | 55 | −1.660 | 31.250 | −16.378 | 1.00 | 57.39 | O |
| ATOM | 8727 | C | GLU | C | 55 | −1.635 | 29.981 | −11.451 | 1.00 | 30.42 | C |
| ATOM | 8728 | O | GLU | C | 55 | −2.223 | 30.071 | −10.381 | 1.00 | 31.41 | O |
| ATOM | 8729 | N | GLY | C | 56 | −0.898 | 28.924 | −11.793 | 1.00 | 27.72 | N |
| ATOM | 8731 | CA | GLY | C | 56 | −0.623 | 27.860 | −10.847 | 1.00 | 23.83 | C |
| ATOM | 8734 | C | GLY | C | 56 | 0.138 | 28.354 | −9.633 | 1.00 | 29.85 | C |
| ATOM | 8735 | O | GLY | C | 56 | 0.224 | 27.696 | −8.596 | 1.00 | 35.12 | O |
| ATOM | 8736 | N | PHE | C | 57 | 0.771 | 29.509 | −9.747 | 1.00 | 26.52 | N |
| ATOM | 8738 | CA | PHE | C | 57 | 1.606 | 29.943 | −8.643 | 1.00 | 28.37 | C |
| ATOM | 8740 | CB | PHE | C | 57 | 2.859 | 30.691 | −9.176 | 1.00 | 28.12 | C |
| ATOM | 8743 | CG | PHE | C | 57 | 3.889 | 29.795 | −9.860 | 1.00 | 27.28 | C |
| ATOM | 8744 | CD1 | PHE | C | 57 | 3.779 | 29.505 | −11.207 | 1.00 | 25.98 | C |
| ATOM | 8746 | CE1 | PHE | C | 57 | 4.753 | 28.747 | −11.876 | 1.00 | 31.88 | C |
| ATOM | 8748 | CZ | PHE | C | 57 | 5.815 | 28.257 | −11.172 | 1.00 | 24.24 | C |
| ATOM | 8750 | CE2 | PHE | C | 57 | 5.892 | 28.473 | −9.785 | 1.00 | 35.58 | C |
| ATOM | 8752 | CD2 | PHE | C | 57 | 4.993 | 29.309 | −9.166 | 1.00 | 23.36 | C |
| ATOM | 8754 | C | PHE | C | 57 | 0.745 | 30.857 | −7.752 | 1.00 | 26.54 | C |
| ATOM | 8755 | O | PHE | C | 57 | −0.051 | 31.643 | −8.241 | 1.00 | 32.89 | O |
| ATOM | 8756 | N | ASP | C | 58 | 0.959 | 30.808 | −6.445 | 1.00 | 29.25 | N |
| ATOM | 8758 | CA | ASP | C | 58 | 0.184 | 31.639 | −5.519 | 1.00 | 32.01 | C |
| ATOM | 8760 | CB | ASP | C | 58 | 0.486 | 31.220 | −4.096 | 1.00 | 28.69 | C |
| ATOM | 8763 | CG | ASP | C | 58 | −0.313 | 32.022 | −3.080 | 1.00 | 34.04 | C |
| ATOM | 8764 | OD1 | ASP | C | 58 | −1.532 | 32.187 | −3.296 | 1.00 | 44.20 | O |
| ATOM | 8765 | OD2 | ASP | C | 58 | 0.249 | 32.579 | −2.104 | 1.00 | 39.10 | O |
| ATOM | 8766 | C | ASP | C | 58 | 0.425 | 33.139 | −5.716 | 1.00 | 42.70 | C |
| ATOM | 8767 | O | ASP | C | 58 | −0.505 | 33.964 | −5.725 | 1.00 | 38.45 | O |
| ATOM | 8768 | N | THR | C | 59 | 1.699 | 33.438 | −5.975 | 1.00 | 49.73 | N |
| ATOM | 8770 | CA | THR | C | 59 | 2.189 | 34.767 | −6.363 | 1.00 | 41.74 | C |
| ATOM | 8772 | CB | THR | C | 59 | 2.877 | 35.358 | −5.116 | 1.00 | 45.77 | C |
| ATOM | 8774 | OG1 | THR | C | 59 | 1.955 | 36.168 | −4.356 | 1.00 | 35.90 | O |
| ATOM | 8776 | CG2 | THR | C | 59 | 4.025 | 36.238 | −5.463 | 1.00 | 43.71 | C |
| ATOM | 8780 | C | THR | C | 59 | 3.154 | 34.646 | −7.587 | 1.00 | 40.78 | C |
| ATOM | 8781 | O | THR | C | 59 | 3.965 | 33.710 | −7.723 | 1.00 | 37.82 | O |
| ATOM | 8782 | N | TYR | C | 60 | 3.031 | 35.579 | −8.518 | 1.00 | 36.74 | N |
| ATOM | 8784 | CA | TYR | C | 60 | 3.759 | 35.521 | −9.768 | 1.00 | 36.70 | C |
| ATOM | 8786 | CB | TYR | C | 60 | 3.022 | 34.688 | −10.795 | 1.00 | 36.17 | C |
| ATOM | 8789 | CG | TYR | C | 60 | 3.938 | 34.234 | −11.903 | 1.00 | 33.52 | C |
| ATOM | 8790 | CD1 | TYR | C | 60 | 4.680 | 33.104 | −11.759 | 1.00 | 26.97 | C |
| ATOM | 8792 | CE1 | TYR | C | 60 | 5.525 | 32.692 | −12.731 | 1.00 | 27.91 | C |
| ATOM | 8794 | CZ | TYR | C | 60 | 5.690 | 33.433 | −13.864 | 1.00 | 28.01 | C |
| ATOM | 8795 | OH | TYR | C | 60 | 6.539 | 32.959 | −14.844 | 1.00 | 30.63 | O |
| ATOM | 8797 | CE2 | TYR | C | 60 | 4.965 | 34.581 | −14.052 | 1.00 | 34.05 | C |
| ATOM | 8799 | CD2 | TYR | C | 60 | 4.118 | 34.987 | −13.055 | 1.00 | 46.49 | C |
| ATOM | 8801 | C | TYR | C | 60 | 4.137 | 36.871 | −10.373 | 1.00 | 39.86 | C |
| ATOM | 8802 | O | TYR | C | 60 | 3.323 | 37.782 | −10.484 | 1.00 | 37.57 | O |
| ATOM | 8803 | N | ARG | C | 61 | 5.414 | 36.970 | −10.745 | 1.00 | 42.25 | N |
| ATOM | 8805 | CA | ARG | C | 61 | 6.004 | 38.232 | −11.144 | 1.00 | 30.18 | C |
| ATOM | 8807 | CB | ARG | C | 61 | 6.539 | 39.003 | −9.941 | 1.00 | 33.51 | C |
| ATOM | 8810 | CG | ARG | C | 61 | 6.852 | 40.439 | −10.292 | 1.00 | 36.23 | C |
| ATOM | 8813 | CD | ARG | C | 61 | 7.432 | 41.319 | −9.184 | 1.00 | 35.74 | C |
| ATOM | 8816 | NE | ARG | C | 61 | 8.258 | 42.410 | −9.725 | 1.00 | 45.98 | N |
| ATOM | 8818 | CZ | ARG | C | 61 | 8.781 | 43.388 | −8.985 | 1.00 | 44.44 | C |
| ATOM | 8819 | NH1 | ARG | C | 61 | 8.518 | 43.465 | −7.695 | 1.00 | 49.73 | N |
| ATOM | 8822 | NH2 | ARG | C | 61 | 9.544 | 44.317 | −9.537 | 1.00 | 51.66 | N |
| ATOM | 8825 | C | ARG | C | 61 | 7.092 | 38.039 | −12.177 | 1.00 | 36.10 | C |
| ATOM | 8826 | O | ARG | C | 61 | 8.102 | 37.378 | −11.950 | 1.00 | 40.81 | O |
| ATOM | 8827 | N | CYS | C | 62 | 6.882 | 38.648 | −13.328 | 1.00 | 35.89 | N |
| ATOM | 8829 | CA | CYS | C | 62 | 7.845 | 38.609 | −14.396 | 1.00 | 41.53 | C |
| ATOM | 8831 | CB | CYS | C | 62 | 7.534 | 37.450 | −15.314 | 1.00 | 32.38 | C |
| ATOM | 8834 | SG | CYS | C | 62 | 8.979 | 37.074 | −16.336 | 1.00 | 46.50 | S |
| ATOM | 8835 | C | CYS | C | 62 | 7.867 | 39.940 | −15.165 | 1.00 | 45.76 | C |
| ATOM | 8836 | O | CYS | C | 62 | 7.001 | 40.197 | −15.996 | 1.00 | 47.81 | O |
| ATOM | 8837 | N | ASP | C | 63 | 8.844 | 40.787 | −14.843 | 1.00 | 43.37 | N |
| ATOM | 8839 | CA | ASP | C | 63 | 9.053 | 42.073 | −15.517 | 1.00 | 48.62 | C |
| ATOM | 8841 | CB | ASP | C | 63 | 10.096 | 42.917 | −14.743 | 1.00 | 40.14 | C |
| ATOM | 8844 | CG | ASP | C | 63 | 9.634 | 43.221 | −13.315 | 1.00 | 46.44 | C |
| ATOM | 8845 | OD1 | ASP | C | 63 | 8.465 | 42.905 | −13.001 | 1.00 | 57.36 | O |
| ATOM | 8846 | OD2 | ASP | C | 63 | 10.345 | 43.695 | −12.403 | 1.00 | 48.95 | O |
| ATOM | 8847 | C | ASP | C | 63 | 9.401 | 41.934 | −17.017 | 1.00 | 42.71 | C |
| ATOM | 8848 | O | ASP | C | 63 | 8.871 | 42.654 | −17.857 | 1.00 | 47.44 | O |
| ATOM | 8849 | N | ARG | C | 64 | 10.228 | 40.961 | −17.358 | 1.00 | 38.97 | N |
| ATOM | 8851 | CA | ARG | C | 64 | 10.565 | 40.701 | −18.743 | 1.00 | 35.38 | C |
| ATOM | 8853 | CB | ARG | C | 64 | 11.700 | 41.633 | −19.135 | 1.00 | 36.74 | C |
| ATOM | 8856 | CG | ARG | C | 64 | 12.930 | 41.442 | −18.305 | 1.00 | 38.08 | C |
| ATOM | 8859 | CD | ARG | C | 64 | 14.181 | 42.066 | −18.951 | 1.00 | 61.37 | C |
| ATOM | 8862 | NE | ARG | C | 64 | 14.485 | 43.344 | −18.304 | 1.00 | 87.32 | N |
| ATOM | 8864 | CZ | ARG | C | 64 | 15.700 | 43.859 | −18.162 | 1.00 | 97.74 | C |
| ATOM | 8865 | NH1 | ARG | C | 64 | 16.760 | 43.225 | −18.661 | 1.00 | 93.68 | N |
| ATOM | 8868 | NH2 | ARG | C | 64 | 15.844 | 45.022 | −17.527 | 1.00 | 90.98 | N |
| ATOM | 8871 | C | ARG | C | 64 | 11.005 | 39.247 | −18.915 | 1.00 | 38.05 | C |
| ATOM | 8872 | O | ARG | C | 64 | 11.387 | 38.580 | −17.951 | 1.00 | 41.42 | O |
| ATOM | 8873 | N | ASN | C | 65 | 10.940 | 38.772 | −20.150 | 1.00 | 42.33 | N |
| ATOM | 8875 | CA | ASN | C | 65 | 11.378 | 37.440 | −20.502 | 1.00 | 48.85 | C |
| ATOM | 8877 | CB | ASN | C | 65 | 11.295 | 37.303 | −22.017 | 1.00 | 47.01 | C |
| ATOM | 8880 | CG | ASN | C | 65 | 11.251 | 35.862 | −22.471 | 1.00 | 77.65 | C |
| ATOM | 8881 | OD1 | ASN | C | 65 | 10.343 | 35.104 | −22.106 | 1.00 | 97.02 | O |
| ATOM | 8882 | ND2 | ASN | C | 65 | 12.232 | 35.473 | −23.283 | 1.00 | 91.75 | N |
| ATOM | 8885 | C | ASN | C | 65 | 12.802 | 37.182 | −20.028 | 1.00 | 52.63 | C |
| ATOM | 8886 | O | ASN | C | 65 | 13.708 | 37.910 | −20.408 | 1.00 | 57.18 | O |
| ATOM | 8887 | N | LEU | C | 66 | 12.994 | 36.164 | −19.188 | 1.00 | 53.72 | N |
| ATOM | 8889 | CA | LEU | C | 66 | 14.321 | 35.621 | −18.895 | 1.00 | 46.07 | C |
| ATOM | 8891 | CB | LEU | C | 66 | 14.512 | 35.396 | −17.397 | 1.00 | 44.45 | C |
| ATOM | 8894 | CG | LEU | C | 66 | 14.284 | 36.546 | −16.412 | 1.00 | 54.59 | C |
| ATOM | 8896 | CD1 | LEU | C | 66 | 14.785 | 36.138 | −15.040 | 1.00 | 47.51 | C |
| ATOM | 8900 | CD2 | LEU | C | 66 | 14.954 | 37.833 | −16.846 | 1.00 | 56.07 | C |
| ATOM | 8904 | C | LEU | C | 66 | 14.549 | 34.277 | −19.555 | 1.00 | 46.03 | C |
| ATOM | 8905 | O | LEU | C | 66 | 13.614 | 33.504 | −19.722 | 1.00 | 48.05 | O |
| ATOM | 8906 | N | ALA | C | 67 | 15.818 | 33.999 | −19.854 | 1.00 | 38.91 | N |
| ATOM | 8908 | CA | ALA | C | 67 | 16.327 | 32.658 | −20.136 | 1.00 | 40.20 | C |
| ATOM | 8910 | CB | ALA | C | 67 | 16.886 | 32.609 | −21.503 | 1.00 | 40.91 | C |
| ATOM | 8914 | C | ALA | C | 67 | 17.427 | 32.280 | −19.143 | 1.00 | 47.64 | C |
| ATOM | 8915 | O | ALA | C | 67 | 18.486 | 32.903 | −19.102 | 1.00 | 42.31 | O |
| ATOM | 8916 | N | MET | C | 68 | 17.188 | 31.266 | −18.321 | 1.00 | 48.16 | N |
| ATOM | 8918 | CA | MET | C | 68 | 18.188 | 30.879 | −17.342 | 1.00 | 45.03 | C |
| ATOM | 8920 | CB | MET | C | 68 | 17.592 | 30.784 | −15.940 | 1.00 | 48.54 | C |
| ATOM | 8923 | CG | MET | C | 68 | 16.675 | 31.910 | −15.563 | 1.00 | 59.18 | C |
| ATOM | 8926 | SD | MET | C | 68 | 16.212 | 31.729 | −13.821 | 1.00 | 58.37 | S |
| ATOM | 8927 | CE | MET | C | 68 | 15.158 | 30.458 | −13.955 | 1.00 | 59.87 | C |
| ATOM | 8931 | C | MET | C | 68 | 18.635 | 29.508 | −17.716 | 1.00 | 42.80 | C |
| ATOM | 8932 | O | MET | C | 68 | 17.805 | 28.597 | −17.774 | 1.00 | 41.12 | O |
| ATOM | 8933 | N | GLY | C | 69 | 19.945 | 29.365 | −17.867 | 1.00 | 40.10 | N |
| ATOM | 8935 | CA | GLY | C | 69 | 20.550 | 28.096 | −18.180 | 1.00 | 40.41 | C |
| ATOM | 8938 | C | GLY | C | 69 | 20.963 | 27.461 | −16.870 | 1.00 | 41.82 | C |
| ATOM | 8939 | O | GLY | C | 69 | 21.531 | 28.135 | −16.045 | 1.00 | 38.25 | O |
| ATOM | 8940 | N | VAL | C | 70 | 20.686 | 26.172 | −16.697 | 1.00 | 39.83 | N |
| ATOM | 8942 | CA | VAL | C | 70 | 20.519 | 25.574 | −15.378 | 1.00 | 35.18 | C |
| ATOM | 8944 | CB | VAL | C | 70 | 18.987 | 25.604 | −14.987 | 1.00 | 47.50 | C |
| ATOM | 8946 | CG1 | VAL | C | 70 | 18.638 | 24.639 | −13.870 | 1.00 | 38.24 | C |
| ATOM | 8950 | CG2 | VAL | C | 70 | 18.533 | 27.016 | −14.642 | 1.00 | 42.46 | C |
| ATOM | 8954 | C | VAL | C | 70 | 21.045 | 24.122 | −15.408 | 1.00 | 36.80 | C |
| ATOM | 8955 | O | VAL | C | 70 | 20.723 | 23.341 | −16.279 | 1.00 | 41.01 | O |
| ATOM | 8956 | N | ASN | C | 71 | 21.911 | 23.766 | −14.477 | 1.00 | 32.40 | N |
| ATOM | 8958 | CA | ASN | C | 71 | 22.309 | 22.384 | −14.300 | 1.00 | 33.41 | C |
| ATOM | 8960 | CB | ASN | C | 71 | 23.651 | 22.375 | −13.505 | 1.00 | 33.33 | C |
| ATOM | 8963 | CG | ASN | C | 71 | 24.292 | 21.016 | −13.463 | 1.00 | 43.42 | C |
| ATOM | 8964 | OD1 | ASN | C | 71 | 23.645 | 20.001 | −13.215 | 1.00 | 38.46 | O |
| ATOM | 8965 | ND2 | ASN | C | 71 | 25.594 | 20.980 | −13.742 | 1.00 | 84.62 | N |
| ATOM | 8968 | C | ASN | C | 71 | 21.210 | 21.730 | −13.465 | 1.00 | 33.61 | C |
| ATOM | 8969 | O | ASN | C | 71 | 21.050 | 22.138 | −12.329 | 1.00 | 33.78 | O |
| ATOM | 8970 | N | LEU | C | 72 | 20.499 | 20.718 | −13.976 | 1.00 | 40.32 | N |
| ATOM | 8972 | CA | LEU | C | 72 | 19.309 | 20.197 | −13.302 | 1.00 | 37.06 | C |
| ATOM | 8974 | CB | LEU | C | 72 | 18.403 | 19.377 | −14.240 | 1.00 | 34.91 | C |
| ATOM | 8977 | CG | LEU | C | 72 | 17.951 | 20.007 | −15.562 | 1.00 | 39.55 | C |
| ATOM | 8979 | CD1 | LEU | C | 72 | 16.945 | 19.136 | −16.254 | 1.00 | 33.86 | C |
| ATOM | 8983 | CD2 | LEU | C | 72 | 17.379 | 21.359 | −15.368 | 1.00 | 38.53 | C |
| ATOM | 8987 | C | LEU | C | 72 | 19.760 | 19.282 | −12.172 | 1.00 | 44.60 | C |
| ATOM | 8988 | O | LEU | C | 72 | 18.976 | 19.026 | −11.255 | 1.00 | 32.43 | O |
| ATOM | 8989 | N | THR | C | 73 | 20.967 | 18.710 | −12.277 | 1.00 | 39.92 | N |
| ATOM | 8991 | CA | THR | C | 73 | 21.502 | 17.858 | −11.210 | 1.00 | 31.80 | C |
| ATOM | 8993 | CB | THR | C | 73 | 22.771 | 17.026 | −11.648 | 1.00 | 48.29 | C |
| ATOM | 8995 | OG1 | THR | C | 73 | 22.476 | 16.226 | −12.806 | 1.00 | 51.77 | O |
| ATOM | 8997 | CG2 | THR | C | 73 | 23.164 | 15.967 | −10.572 | 1.00 | 31.66 | C |
| ATOM | 9001 | C | THR | C | 73 | 21.812 | 18.733 | −9.995 | 1.00 | 30.50 | C |
| ATOM | 9002 | O | THR | C | 73 | 21.422 | 18.410 | −8.881 | 1.00 | 39.82 | O |
| ATOM | 9003 | N | SER | C | 74 | 22.309 | 19.940 | −10.213 | 1.00 | 30.68 | N |
| ATOM | 9005 | CA | SER | C | 74 | 22.546 | 20.815 | −9.088 | 1.00 | 33.64 | C |
| ATOM | 9007 | CB | SER | C | 74 | 23.398 | 22.023 | −9.500 | 1.00 | 27.87 | C |
| ATOM | 9010 | OG | SER | C | 74 | 24.514 | 21.607 | −10.276 | 1.00 | 54.27 | O |
| ATOM | 9012 | C | SER | C | 74 | 21.193 | 21.293 | −8.531 | 1.00 | 32.40 | C |
| ATOM | 9013 | O | SER | C | 74 | 21.031 | 21.444 | −7.328 | 1.00 | 28.18 | O |
| ATOM | 9014 | N | MET | C | 75 | 20.265 | 21.644 | −9.406 | 1.00 | 30.42 | N |
| ATOM | 9016 | CA | MET | C | 75 | 18.946 | 22.114 | −8.956 | 1.00 | 36.25 | C |
| ATOM | 9018 | CB | MET | C | 75 | 18.078 | 22.551 | −10.138 | 1.00 | 38.35 | C |
| ATOM | 9021 | CG | MET | C | 75 | 16.753 | 23.234 | −9.678 | 1.00 | 36.05 | C |
| ATOM | 9024 | SD | MET | C | 75 | 16.088 | 24.213 | −11.050 | 1.00 | 34.14 | S |
| ATOM | 9025 | CE | MET | C | 75 | 14.751 | 25.025 | −10.202 | 1.00 | 33.72 | C |
| ATOM | 9029 | C | MET | C | 75 | 18.170 | 21.079 | −8.103 | 1.00 | 24.02 | C |
| ATOM | 9030 | O | MET | C | 75 | 17.692 | 21.408 | −7.039 | 1.00 | 27.67 | O |
| ATOM | 9031 | N | SER | C | 76 | 18.174 | 19.822 | −8.524 | 1.00 | 24.28 | N |
| ATOM | 9033 | CA | SER | C | 76 | 17.635 | 18.681 | −7.786 | 1.00 | 28.40 | C |
| ATOM | 9035 | CB | SER | C | 76 | 17.859 | 17.380 | −8.580 | 1.00 | 23.01 | C |
| ATOM | 9038 | OG | SER | C | 76 | 17.099 | 16.329 | −8.013 | 1.00 | 44.73 | O |
| ATOM | 9040 | C | SER | C | 76 | 18.245 | 18.488 | −6.418 | 1.00 | 34.78 | C |
| ATOM | 9041 | O | SER | C | 76 | 17.534 | 18.183 | −5.444 | 1.00 | 39.91 | O |
| ATOM | 9042 | N | LYS | C | 77 | 19.560 | 18.677 | −6.351 | 1.00 | 24.68 | N |
| ATOM | 9044 | CA | LYS | C | 77 | 20.299 | 18.472 | −5.131 | 1.00 | 24.59 | C |
| ATOM | 9046 | CB | LYS | C | 77 | 21.784 | 18.800 | −5.388 | 1.00 | 33.44 | C |
| ATOM | 9049 | CG | LYS | C | 77 | 22.784 | 17.787 | −4.877 | 1.00 | 51.64 | C |
| ATOM | 9052 | CD | LYS | C | 77 | 23.384 | 16.943 | −5.991 | 1.00 | 58.27 | C |
| ATOM | 9055 | CE | LYS | C | 77 | 24.764 | 16.403 | −5.594 | 1.00 | 76.87 | C |
| ATOM | 9058 | NZ | LYS | C | 77 | 24.942 | 16.170 | −4.120 | 1.00 | 69.35 | N |
| ATOM | 9062 | C | LYS | C | 77 | 19.791 | 19.491 | −4.147 | 1.00 | 26.39 | C |
| ATOM | 9063 | O | LYS | C | 77 | 19.631 | 19.142 | −2.990 | 1.00 | 33.02 | O |
| ATOM | 9064 | N | ILE | C | 78 | 19.759 | 20.769 | −4.569 | 1.00 | 24.98 | N |
| ATOM | 9066 | CA | ILE | C | 78 | 19.256 | 21.846 | −3.744 | 1.00 | 27.42 | C |
| ATOM | 9068 | CB | ILE | C | 78 | 19.402 | 23.182 | −4.475 | 1.00 | 23.82 | C |
| ATOM | 9070 | CG1 | ILE | C | 78 | 20.888 | 23.553 | −4.592 | 1.00 | 37.61 | C |
| ATOM | 9073 | CD1 | ILE | C | 78 | 21.119 | 24.625 | −5.569 | 1.00 | 25.50 | C |
| ATOM | 9077 | CG2 | ILE | C | 78 | 18.610 | 24.271 | −3.776 | 1.00 | 27.36 | C |
| ATOM | 9081 | C | ILE | C | 78 | 17.798 | 21.597 | −3.332 | 1.00 | 29.37 | C |
| ATOM | 9082 | O | ILE | C | 78 | 17.420 | 21.813 | −2.175 | 1.00 | 33.22 | O |
| ATOM | 9083 | N | LEU | C | 79 | 16.965 | 21.164 | −4.271 | 1.00 | 32.00 | N |
| ATOM | 9085 | CA | LEU | C | 79 | 15.567 | 20.867 | −3.909 | 1.00 | 34.88 | C |
| ATOM | 9087 | CB | LEU | C | 79 | 14.752 | 20.684 | −5.163 | 1.00 | 24.20 | C |
| ATOM | 9090 | CG | LEU | C | 79 | 14.282 | 22.020 | −5.741 | 1.00 | 30.61 | C |
| ATOM | 9092 | CD1 | LEU | C | 79 | 14.030 | 21.872 | −7.177 | 1.00 | 25.86 | C |
| ATOM | 9096 | CD2 | LEU | C | 79 | 12.989 | 22.515 | −5.085 | 1.00 | 26.07 | C |
| ATOM | 9100 | C | LEU | C | 79 | 15.313 | 19.700 | −2.930 | 1.00 | 33.78 | C |
| ATOM | 9101 | O | LEU | C | 79 | 14.264 | 19.676 | −2.252 | 1.00 | 40.17 | O |
| ATOM | 9102 | N | LYS | C | 80 | 16.274 | 18.771 | −2.825 | 1.00 | 35.28 | N |
| ATOM | 9104 | CA | LYS | C | 80 | 16.286 | 17.706 | −1.810 | 1.00 | 35.04 | C |
| ATOM | 9106 | CB | LYS | C | 80 | 17.448 | 16.734 | −2.099 | 1.00 | 43.11 | C |
| ATOM | 9109 | CG | LYS | C | 80 | 17.002 | 15.445 | −2.822 | 1.00 | 50.42 | C |
| ATOM | 9112 | CD | LYS | C | 80 | 18.084 | 14.870 | −3.745 | 1.00 | 66.08 | C |
| ATOM | 9115 | CE | LYS | C | 80 | 17.466 | 14.024 | −4.864 | 1.00 | 74.08 | C |
| ATOM | 9118 | NZ | LYS | C | 80 | 18.242 | 12.791 | −5.206 | 1.00 | 86.43 | N |
| ATOM | 9122 | C | LYS | C | 80 | 16.406 | 18.253 | −0.371 | 1.00 | 32.48 | C |
| ATOM | 9123 | O | LYS | C | 80 | 15.969 | 17.649 | 0.594 | 1.00 | 38.05 | O |
| ATOM | 9124 | N | CYS | C | 81 | 16.874 | 19.481 | −0.262 | 1.00 | 35.25 | N |
| ATOM | 9126 | CA | CYS | C | 81 | 16.840 | 20.245 | 0.984 | 1.00 | 29.19 | C |
| ATOM | 9128 | CB | CYS | C | 81 | 17.861 | 21.354 | 0.921 | 1.00 | 31.43 | C |
| ATOM | 9131 | SG | CYS | C | 81 | 19.524 | 20.755 | 0.460 | 1.00 | 33.56 | S |
| ATOM | 9132 | C | CYS | C | 81 | 15.511 | 20.886 | 1.298 | 1.00 | 39.25 | C |
| ATOM | 9133 | O | CYS | C | 81 | 15.327 | 21.428 | 2.398 | 1.00 | 42.64 | O |
| ATOM | 9134 | N | ALA | C | 82 | 14.587 | 20.836 | 0.343 | 1.00 | 38.13 | N |
| ATOM | 9136 | CA | ALA | C | 82 | 13.217 | 21.262 | 0.599 | 1.00 | 32.44 | C |
| ATOM | 9138 | CB | ALA | C | 82 | 12.604 | 21.759 | −0.670 | 1.00 | 31.07 | C |
| ATOM | 9142 | C | ALA | C | 82 | 12.366 | 20.127 | 1.151 | 1.00 | 22.93 | C |
| ATOM | 9143 | O | ALA | C | 82 | 12.427 | 19.017 | 0.644 | 1.00 | 27.49 | O |
| ATOM | 9144 | N | GLY | C | 83 | 11.478 | 20.464 | 2.080 | 1.00 | 30.72 | N |
| ATOM | 9146 | CA | GLY | C | 83 | 10.390 | 19.580 | 2.504 | 1.00 | 21.75 | C |
| ATOM | 9149 | C | GLY | C | 83 | 9.289 | 19.506 | 1.488 | 1.00 | 18.56 | C |
| ATOM | 9150 | O | GLY | C | 83 | 9.078 | 20.446 | 0.738 | 1.00 | 28.23 | O |
| ATOM | 9151 | N | ASN | C | 84 | 8.555 | 18.410 | 1.469 | 1.00 | 31.73 | N |
| ATOM | 9153 | CA | ASN | C | 84 | 7.376 | 18.273 | 0.610 | 1.00 | 32.14 | C |
| ATOM | 9155 | CB | ASN | C | 84 | 6.790 | 16.883 | 0.810 | 1.00 | 38.19 | C |
| ATOM | 9158 | CG | ASN | C | 84 | 7.539 | 15.845 | 0.002 | 1.00 | 35.02 | C |
| ATOM | 9159 | OD1 | ASN | C | 84 | 7.743 | 16.027 | −1.196 | 1.00 | 40.53 | O |
| ATOM | 9160 | ND2 | ASN | C | 84 | 8.051 | 14.826 | 0.667 | 1.00 | 51.45 | N |
| ATOM | 9163 | C | ASN | C | 84 | 6.275 | 19.310 | 0.768 | 1.00 | 24.89 | C |
| ATOM | 9164 | O | ASN | C | 84 | 5.653 | 19.733 | −0.220 | 1.00 | 33.25 | O |
| ATOM | 9165 | N | GLU | C | 85 | 6.187 | 19.868 | 1.966 | 1.00 | 30.59 | N |
| ATOM | 9167 | CA | GLU | C | 85 | 5.103 | 20.806 | 2.266 | 1.00 | 39.55 | C |
| ATOM | 9169 | CB | GLU | C | 85 | 4.296 | 20.291 | 3.491 | 1.00 | 38.95 | C |
| ATOM | 9172 | CG | GLU | C | 85 | 3.160 | 19.315 | 3.131 | 1.00 | 49.46 | C |
| ATOM | 9175 | CD | GLU | C | 85 | 3.564 | 17.838 | 3.061 | 1.00 | 71.91 | C |
| ATOM | 9176 | OE1 | GLU | C | 85 | 4.241 | 17.340 | 3.998 | 1.00 | 70.35 | O |
| ATOM | 9177 | OE2 | GLU | C | 85 | 3.163 | 17.150 | 2.087 | 1.00 | 73.52 | O |
| ATOM | 9178 | C | GLU | C | 85 | 5.613 | 22.233 | 2.446 | 1.00 | 34.51 | C |
| ATOM | 9179 | O | GLU | C | 85 | 4.849 | 23.158 | 2.786 | 1.00 | 32.17 | O |
| ATOM | 9180 | N | ASP | C | 86 | 6.886 | 22.430 | 2.104 | 1.00 | 31.30 | N |
| ATOM | 9182 | CA | ASP | C | 86 | 7.434 | 23.783 | 2.029 | 1.00 | 29.32 | C |
| ATOM | 9184 | CB | ASP | C | 86 | 8.967 | 23.779 | 1.818 | 1.00 | 30.27 | C |
| ATOM | 9187 | CG | ASP | C | 86 | 9.752 | 23.295 | 3.040 | 1.00 | 37.80 | C |
| ATOM | 9188 | OD1 | ASP | C | 86 | 9.151 | 22.764 | 4.013 | 1.00 | 52.02 | O |
| ATOM | 9189 | OD2 | ASP | C | 86 | 11.000 | 23.360 | 3.070 | 1.00 | 46.21 | O |
| ATOM | 9190 | C | ASP | C | 86 | 6.764 | 24.646 | 0.958 | 1.00 | 23.92 | C |
| ATOM | 9191 | O | ASP | C | 86 | 6.413 | 24.183 | −0.124 | 1.00 | 21.26 | O |
| ATOM | 9192 | N | ILE | C | 87 | 6.693 | 25.947 | 1.241 | 1.00 | 22.84 | N |
| ATOM | 9194 | CA | ILE | C | 87 | 6.418 | 26.884 | 0.193 | 1.00 | 30.41 | C |
| ATOM | 9196 | CB | ILE | C | 87 | 5.675 | 28.120 | 0.789 | 1.00 | 25.12 | C |
| ATOM | 9198 | CG1 | ILE | C | 87 | 4.228 | 27.763 | 1.191 | 1.00 | 35.15 | C |
| ATOM | 9201 | CD1 | ILE | C | 87 | 3.968 | 27.505 | 2.588 | 1.00 | 33.48 | C |
| ATOM | 9205 | CG2 | ILE | C | 87 | 5.513 | 29.150 | −0.278 | 1.00 | 22.96 | C |
| ATOM | 9209 | C | ILE | C | 87 | 7.792 | 27.243 | −0.430 | 1.00 | 29.38 | C |
| ATOM | 9210 | O | ILE | C | 87 | 8.659 | 27.850 | 0.220 | 1.00 | 22.43 | O |
| ATOM | 9211 | N | ILE | C | 88 | 7.924 | 26.977 | −1.719 | 1.00 | 30.71 | N |
| ATOM | 9213 | CA | ILE | C | 88 | 9.113 | 27.361 | −2.466 | 1.00 | 33.36 | C |
| ATOM | 9215 | CB | ILE | C | 88 | 9.605 | 26.196 | −3.347 | 1.00 | 31.20 | C |
| ATOM | 9217 | CG1 | ILE | C | 88 | 9.919 | 24.963 | −2.478 | 1.00 | 30.51 | C |
| ATOM | 9220 | CD1 | ILE | C | 88 | 10.244 | 23.607 | −3.233 | 1.00 | 22.60 | C |
| ATOM | 9224 | CG2 | ILE | C | 88 | 10.786 | 26.669 | −4.218 | 1.00 | 26.62 | C |
| ATOM | 9228 | C | ILE | C | 88 | 8.831 | 28.611 | −3.308 | 1.00 | 30.37 | C |
| ATOM | 9229 | O | ILE | C | 88 | 8.036 | 28.637 | −4.234 | 1.00 | 29.67 | O |
| ATOM | 9230 | N | THR | C | 89 | 9.594 | 29.626 | −2.988 | 1.00 | 26.46 | N |
| ATOM | 9232 | CA | THR | C | 89 | 9.780 | 30.803 | −3.814 | 1.00 | 29.36 | C |
| ATOM | 9234 | CB | THR | C | 89 | 9.728 | 31.996 | −2.855 | 1.00 | 21.66 | C |
| ATOM | 9236 | OG1 | THR | C | 89 | 8.433 | 32.021 | −2.211 | 1.00 | 37.79 | O |
| ATOM | 9238 | CG2 | THR | C | 89 | 9.810 | 33.294 | −3.671 | 1.00 | 40.21 | C |
| ATOM | 9242 | C | THR | C | 89 | 11.053 | 30.870 | −4.713 | 1.00 | 30.60 | C |
| ATOM | 9243 | O | THR | C | 89 | 12.195 | 30.882 | −4.235 | 1.00 | 35.22 | O |
| ATOM | 9244 | N | LEU | C | 90 | 10.848 | 30.936 | −6.033 | 1.00 | 37.81 | N |
| ATOM | 9246 | CA | LEU | C | 90 | 11.925 | 31.189 | −7.001 | 1.00 | 22.21 | C |
| ATOM | 9248 | CB | LEU | C | 90 | 11.676 | 30.471 | −8.312 | 1.00 | 24.55 | C |
| ATOM | 9251 | CG | LEU | C | 90 | 11.475 | 28.967 | −8.224 | 1.00 | 23.04 | C |
| ATOM | 9253 | CD1 | LEU | C | 90 | 11.175 | 28.453 | −9.615 | 1.00 | 33.23 | C |
| ATOM | 9257 | CD2 | LEU | C | 90 | 12.751 | 28.379 | −7.651 | 1.00 | 37.39 | C |
| ATOM | 9261 | C | LEU | C | 90 | 12.070 | 32.657 | −7.336 | 1.00 | 32.71 | C |
| ATOM | 9262 | O | LEU | C | 90 | 11.076 | 33.328 | −7.584 | 1.00 | 33.50 | O |
| ATOM | 9263 | N | ARG | C | 91 | 13.317 | 33.133 | −7.377 | 1.00 | 32.48 | N |
| ATOM | 9265 | CA | ARG | C | 91 | 13.629 | 34.548 | −7.584 | 1.00 | 31.43 | C |
| ATOM | 9267 | CB | ARG | C | 91 | 13.709 | 35.227 | −6.214 | 1.00 | 36.33 | C |
| ATOM | 9270 | CG | ARG | C | 91 | 14.065 | 36.712 | −6.242 | 1.00 | 38.69 | C |
| ATOM | 9273 | CD | ARG | C | 91 | 13.727 | 37.492 | −4.989 | 1.00 | 37.88 | C |
| ATOM | 9276 | NE | ARG | C | 91 | 14.204 | 36.821 | −3.786 | 1.00 | 60.77 | N |
| ATOM | 9278 | CZ | ARG | C | 91 | 13.443 | 36.487 | −2.750 | 1.00 | 65.34 | C |
| ATOM | 9279 | NH1 | ARG | C | 91 | 12.137 | 36.744 | −2.756 | 1.00 | 53.97 | N |
| ATOM | 9282 | NH2 | ARG | C | 91 | 14.008 | 35.911 | −1.694 | 1.00 | 65.24 | N |
| ATOM | 9285 | C | ARG | C | 91 | 14.947 | 34.799 | −8.378 | 1.00 | 40.65 | C |
| ATOM | 9286 | O | ARG | C | 91 | 15.993 | 34.180 | −8.141 | 1.00 | 31.58 | O |
| ATOM | 9287 | N | ALA | C | 92 | 14.858 | 35.640 | −9.401 | 1.00 | 38.50 | N |
| ATOM | 9289 | CA | ALA | C | 92 | 16.035 | 36.138 | −10.108 | 1.00 | 42.48 | C |
| ATOM | 9291 | CB | ALA | C | 92 | 16.260 | 35.350 | −11.361 | 1.00 | 38.08 | C |
| ATOM | 9295 | C | ALA | C | 92 | 15.891 | 37.619 | −10.459 | 1.00 | 46.55 | C |
| ATOM | 9296 | O | ALA | C | 92 | 14.887 | 38.046 | −11.002 | 1.00 | 45.58 | O |
| ATOM | 9297 | N | GLU | C | 93 | 16.929 | 38.398 | −10.204 | 1.00 | 55.13 | N |
| ATOM | 9299 | CA | GLU | C | 93 | 17.044 | 39.699 | −10.846 | 1.00 | 56.34 | C |
| ATOM | 9301 | CB | GLU | C | 93 | 18.196 | 40.499 | −10.247 | 1.00 | 54.54 | C |
| ATOM | 9304 | CG | GLU | C | 93 | 17.794 | 41.274 | −8.988 | 1.00 | 67.71 | C |
| ATOM | 9307 | CD | GLU | C | 93 | 16.664 | 42.303 | −9.187 | 1.00 | 84.90 | C |
| ATOM | 9308 | OE1 | GLU | C | 93 | 16.270 | 42.672 | −10.337 | 1.00 | 67.01 | O |
| ATOM | 9309 | OE2 | GLU | C | 93 | 16.151 | 42.770 | −8.143 | 1.00 | 85.64 | O |
| ATOM | 9310 | C | GLU | C | 93 | 17.141 | 39.565 | −12.363 | 1.00 | 50.61 | C |
| ATOM | 9311 | O | GLU | C | 93 | 17.385 | 38.482 | −12.886 | 1.00 | 52.50 | O |
| ATOM | 9312 | N | ASP | C | 94 | 16.823 | 40.645 | −13.068 | 1.00 | 57.45 | N |
| ATOM | 9314 | CA | ASP | C | 94 | 16.549 | 40.565 | −14.499 | 1.00 | 66.19 | C |
| ATOM | 9316 | CB | ASP | C | 94 | 16.040 | 41.919 | −15.024 | 1.00 | 69.44 | C |
| ATOM | 9319 | CG | ASP | C | 94 | 14.812 | 42.432 | −14.243 | 1.00 | 79.29 | C |
| ATOM | 9320 | OD1 | ASP | C | 94 | 14.819 | 43.606 | −13.801 | 1.00 | 100.71 | O |
| ATOM | 9321 | OD2 | ASP | C | 94 | 13.802 | 41.730 | −13.992 | 1.00 | 58.15 | O |
| ATOM | 9322 | C | ASP | C | 94 | 17.728 | 39.969 | −15.305 | 1.00 | 71.32 | C |
| ATOM | 9323 | O | ASP | C | 94 | 17.620 | 38.863 | −15.826 | 1.00 | 71.46 | O |
| ATOM | 9324 | N | ASN | C | 95 | 18.889 | 40.612 | −15.342 | 1.00 | 74.74 | N |
| ATOM | 9326 | CA | ASN | C | 95 | 20.063 | 39.912 | −15.862 | 1.00 | 80.82 | C |
| ATOM | 9328 | CB | ASN | C | 95 | 20.714 | 40.654 | −17.052 | 1.00 | 84.70 | C |
| ATOM | 9331 | CG | ASN | C | 95 | 20.714 | 42.182 | −16.896 | 1.00 | 93.27 | C |
| ATOM | 9332 | OD1 | ASN | C | 95 | 21.334 | 42.734 | −15.976 | 1.00 | 94.91 | O |
| ATOM | 9333 | ND2 | ASN | C | 95 | 20.053 | 42.871 | −17.828 | 1.00 | 80.42 | N |
| ATOM | 9336 | C | ASN | C | 95 | 21.060 | 39.600 | −14.746 | 1.00 | 79.36 | C |
| ATOM | 9337 | O | ASN | C | 95 | 22.155 | 40.159 | −14.691 | 1.00 | 83.02 | O |
| ATOM | 9338 | N | ALA | C | 96 | 20.654 | 38.726 | −13.829 | 1.00 | 76.80 | N |
| ATOM | 9340 | CA | ALA | C | 96 | 21.568 | 38.196 | −12.821 | 1.00 | 69.32 | C |
| ATOM | 9342 | CB | ALA | C | 96 | 20.957 | 38.295 | −11.427 | 1.00 | 75.23 | C |
| ATOM | 9346 | C | ALA | C | 96 | 21.934 | 36.750 | −13.153 | 1.00 | 57.02 | C |
| ATOM | 9347 | O | ALA | C | 96 | 21.241 | 36.062 | −13.901 | 1.00 | 54.60 | O |
| ATOM | 9348 | N | ASP | C | 97 | 23.059 | 36.297 | −12.629 | 1.00 | 53.84 | N |
| ATOM | 9350 | CA | ASP | C | 97 | 23.611 | 35.022 | −13.068 | 1.00 | 59.67 | C |
| ATOM | 9352 | CB | ASP | C | 97 | 25.123 | 35.126 | −13.394 | 1.00 | 68.32 | C |
| ATOM | 9355 | CG | ASP | C | 97 | 25.537 | 34.322 | −14.657 | 1.00 | 78.66 | C |
| ATOM | 9356 | OD1 | ASP | C | 97 | 25.532 | 34.910 | −15.767 | 1.00 | 86.84 | O |
| ATOM | 9357 | OD2 | ASP | C | 97 | 25.913 | 33.117 | −14.639 | 1.00 | 64.15 | O |
| ATOM | 9358 | C | ASP | C | 97 | 23.364 | 34.055 | −11.934 | 1.00 | 54.08 | C |
| ATOM | 9359 | O | ASP | C | 97 | 24.174 | 33.153 | −11.705 | 1.00 | 61.41 | O |
| ATOM | 9360 | N | THR | C | 98 | 22.245 | 34.249 | −11.224 | 1.00 | 52.64 | N |
| ATOM | 9362 | CA | THR | C | 98 | 21.849 | 33.337 | −10.148 | 1.00 | 51.31 | C |
| ATOM | 9364 | CB | THR | C | 98 | 22.354 | 33.820 | −8.777 | 1.00 | 51.58 | C |
| ATOM | 9366 | OG1 | THR | C | 98 | 22.193 | 35.236 | −8.710 | 1.00 | 44.48 | O |
| ATOM | 9368 | CG2 | THR | C | 98 | 23.833 | 33.632 | −8.624 | 1.00 | 44.03 | C |
| ATOM | 9372 | C | THR | C | 98 | 20.356 | 33.191 | −10.013 | 1.00 | 45.62 | C |
| ATOM | 9373 | O | THR | C | 98 | 19.582 | 34.134 | −10.159 | 1.00 | 43.31 | O |
| ATOM | 9374 | N | LEU | C | 99 | 19.984 | 32.013 | −9.549 | 1.00 | 39.27 | N |
| ATOM | 9376 | CA | LEU | C | 99 | 18.626 | 31.748 | −9.137 | 1.00 | 29.52 | C |
| ATOM | 9378 | CB | LEU | C | 99 | 18.093 | 30.660 | −10.054 | 1.00 | 34.82 | C |
| ATOM | 9381 | CG | LEU | C | 99 | 16.679 | 30.171 | −9.778 | 1.00 | 32.46 | C |
| ATOM | 9383 | CD1 | LEU | C | 99 | 15.659 | 31.195 | −10.248 | 1.00 | 36.71 | C |
| ATOM | 9387 | CD2 | LEU | C | 99 | 16.524 | 28.844 | −10.424 | 1.00 | 38.10 | C |
| ATOM | 9391 | C | LEU | C | 99 | 18.630 | 31.387 | −7.642 | 1.00 | 31.02 | C |
| ATOM | 9392 | O | LEU | C | 99 | 19.362 | 30.509 | −7.176 | 1.00 | 37.51 | O |
| ATOM | 9393 | N | ALA | C | 100 | 17.951 | 32.224 | −6.876 | 1.00 | 31.48 | N |
| ATOM | 9395 | CA | ALA | C | 100 | 17.629 | 31.980 | −5.471 | 1.00 | 30.69 | C |
| ATOM | 9397 | CB | ALA | C | 100 | 17.439 | 33.287 | −4.753 | 1.00 | 24.47 | C |
| ATOM | 9401 | C | ALA | C | 100 | 16.377 | 31.124 | −5.351 | 1.00 | 26.44 | C |
| ATOM | 9402 | O | ALA | C | 100 | 15.499 | 31.192 | −6.206 | 1.00 | 22.97 | O |
| ATOM | 9403 | N | LEU | C | 101 | 16.391 | 30.226 | −4.372 | 1.00 | 30.53 | N |
| ATOM | 9405 | CA | LEU | C | 101 | 15.328 | 29.269 | −4.092 | 1.00 | 29.97 | C |
| ATOM | 9407 | CB | LEU | C | 101 | 15.778 | 27.864 | −4.427 | 1.00 | 31.51 | C |
| ATOM | 9410 | CG | LEU | C | 101 | 16.247 | 27.565 | −5.837 | 1.00 | 33.39 | C |
| ATOM | 9412 | CD1 | LEU | C | 101 | 17.755 | 27.781 | −6.146 | 1.00 | 45.19 | C |
| ATOM | 9416 | CD2 | LEU | C | 101 | 15.844 | 26.160 | −6.173 | 1.00 | 32.48 | C |
| ATOM | 9420 | C | LEU | C | 101 | 15.145 | 29.346 | −2.581 | 1.00 | 37.79 | C |
| ATOM | 9421 | O | LEU | C | 101 | 16.092 | 29.053 | −1.852 | 1.00 | 40.65 | O |
| ATOM | 9422 | N | VAL | C | 102 | 13.978 | 29.826 | −2.141 | 1.00 | 35.34 | N |
| ATOM | 9424 | CA | VAL | C | 102 | 13.645 | 29.998 | −0.726 | 1.00 | 31.12 | C |
| ATOM | 9426 | CB | VAL | C | 102 | 13.050 | 31.364 | −0.482 | 1.00 | 25.78 | C |
| ATOM | 9428 | CG1 | VAL | C | 102 | 12.884 | 31.660 | 0.973 | 1.00 | 30.48 | C |
| ATOM | 9432 | CG2 | VAL | C | 102 | 13.851 | 32.447 | −1.164 | 1.00 | 31.93 | C |
| ATOM | 9436 | C | VAL | C | 102 | 12.595 | 28.956 | −0.354 | 1.00 | 30.65 | C |
| ATOM | 9437 | O | VAL | C | 102 | 11.582 | 28.821 | −1.029 | 1.00 | 25.35 | O |
| ATOM | 9438 | N | PHE | C | 103 | 12.895 | 28.199 | 0.691 | 1.00 | 25.55 | N |
| ATOM | 9440 | CA | PHE | C | 103 | 12.084 | 27.108 | 1.153 | 1.00 | 31.27 | C |
| ATOM | 9442 | CB | PHE | C | 103 | 12.931 | 25.817 | 1.275 | 1.00 | 19.95 | C |
| ATOM | 9445 | CG | PHE | C | 103 | 13.626 | 25.416 | 0.017 | 1.00 | 38.28 | C |
| ATOM | 9446 | CD1 | PHE | C | 103 | 13.159 | 25.826 | −1.215 | 1.00 | 27.28 | C |
| ATOM | 9448 | CE1 | PHE | C | 103 | 13.796 | 25.426 | −2.349 | 1.00 | 32.69 | C |
| ATOM | 9450 | CZ | PHE | C | 103 | 14.929 | 24.642 | −2.280 | 1.00 | 24.71 | C |
| ATOM | 9452 | CE2 | PHE | C | 103 | 15.356 | 24.175 | −1.061 | 1.00 | 22.61 | C |
| ATOM | 9454 | CD2 | PHE | C | 103 | 14.775 | 24.635 | 0.072 | 1.00 | 28.13 | C |
| ATOM | 9456 | C | PHE | C | 103 | 11.636 | 27.579 | 2.530 | 1.00 | 27.73 | C |
| ATOM | 9457 | O | PHE | C | 103 | 12.426 | 27.605 | 3.470 | 1.00 | 39.99 | O |
| ATOM | 9458 | N | GLU | C | 104 | 10.375 | 27.976 | 2.615 | 1.00 | 28.36 | N |
| ATOM | 9460 | CA | GLU | C | 104 | 9.718 | 28.302 | 3.892 | 1.00 | 25.38 | C |
| ATOM | 9462 | CB | GLU | C | 104 | 8.787 | 29.498 | 3.744 | 1.00 | 22.38 | C |
| ATOM | 9465 | CG | GLU | C | 104 | 9.437 | 30.802 | 3.310 | 1.00 | 35.13 | C |
| ATOM | 9468 | CD | GLU | C | 104 | 8.409 | 31.918 | 3.138 | 1.00 | 57.47 | C |
| ATOM | 9469 | OE1 | GLU | C | 104 | 7.226 | 31.585 | 2.952 | 1.00 | 53.26 | O |
| ATOM | 9470 | OE2 | GLU | C | 104 | 8.761 | 33.121 | 3.229 | 1.00 | 65.79 | O |
| ATOM | 9471 | C | GLU | C | 104 | 8.921 | 27.126 | 4.405 | 1.00 | 16.99 | C |
| ATOM | 9472 | O | GLU | C | 104 | 8.051 | 26.600 | 3.709 | 1.00 | 33.92 | O |
| ATOM | 9473 | N | ALA | C | 105 | 9.231 | 26.632 | 5.588 | 1.00 | 34.69 | N |
| ATOM | 9475 | CA | ALA | C | 105 | 8.226 | 25.811 | 6.268 | 1.00 | 47.74 | C |
| ATOM | 9477 | CB | ALA | C | 105 | 8.663 | 25.394 | 7.603 | 1.00 | 47.73 | C |
| ATOM | 9481 | C | ALA | C | 105 | 6.867 | 26.501 | 6.359 | 1.00 | 54.67 | C |
| ATOM | 9482 | O | ALA | C | 105 | 6.704 | 27.695 | 6.081 | 1.00 | 58.07 | O |
| ATOM | 9483 | N | PRO | C | 106 | 5.856 | 25.693 | 6.629 | 1.00 | 67.44 | N |
| ATOM | 9484 | CA | PRO | C | 106 | 4.469 | 26.190 | 6.659 | 1.00 | 66.82 | C |
| ATOM | 9486 | CB | PRO | C | 106 | 3.624 | 24.906 | 6.602 | 1.00 | 68.81 | C |
| ATOM | 9489 | CG | PRO | C | 106 | 4.612 | 23.746 | 6.449 | 1.00 | 67.52 | C |
| ATOM | 9492 | CD | PRO | C | 106 | 5.969 | 24.244 | 6.883 | 1.00 | 65.34 | C |
| ATOM | 9495 | C | PRO | C | 106 | 4.131 | 27.053 | 7.892 | 1.00 | 66.72 | C |
| ATOM | 9496 | O | PRO | C | 106 | 3.206 | 27.884 | 7.805 | 1.00 | 64.75 | O |
| ATOM | 9497 | N | ASN | C | 107 | 4.881 | 26.883 | 8.991 | 1.00 | 59.79 | N |
| ATOM | 9499 | CA | ASN | C | 107 | 4.719 | 27.694 | 10.212 | 1.00 | 58.22 | C |
| ATOM | 9501 | CB | ASN | C | 107 | 4.791 | 26.809 | 11.461 | 1.00 | 55.43 | C |
| ATOM | 9504 | CG | ASN | C | 107 | 6.109 | 26.035 | 11.559 | 1.00 | 60.52 | C |
| ATOM | 9505 | OD1 | ASN | C | 107 | 6.172 | 24.971 | 12.179 | 1.00 | 59.34 | O |
| ATOM | 9506 | ND2 | ASN | C | 107 | 7.163 | 26.569 | 10.949 | 1.00 | 36.63 | N |
| ATOM | 9509 | C | ASN | C | 107 | 5.743 | 28.835 | 10.350 | 1.00 | 57.27 | C |
| ATOM | 9510 | O | ASN | C | 107 | 5.833 | 29.474 | 11.397 | 1.00 | 43.51 | O |
| ATOM | 9511 | N | GLN | C | 108 | 6.527 | 29.048 | 9.297 | 1.00 | 55.96 | N |
| ATOM | 9513 | CA | GLN | C | 108 | 7.605 | 30.062 | 9.225 | 1.00 | 58.84 | C |
| ATOM | 9515 | CB | GLN | C | 108 | 7.050 | 31.478 | 8.929 | 1.00 | 63.99 | C |
| ATOM | 9518 | CG | GLN | C | 108 | 6.225 | 32.147 | 10.030 | 1.00 | 77.01 | C |
| ATOM | 9521 | CD | GLN | C | 108 | 4.927 | 32.773 | 9.512 | 1.00 | 87.45 | C |
| ATOM | 9522 | OE1 | GLN | C | 108 | 3.990 | 33.008 | 10.296 | 1.00 | 50.35 | O |
| ATOM | 9523 | NE2 | GLN | C | 108 | 4.869 | 33.038 | 8.196 | 1.00 | 89.00 | N |
| ATOM | 9526 | C | GLN | C | 108 | 8.731 | 30.087 | 10.278 | 1.00 | 46.54 | C |
| ATOM | 9527 | O | GLN | C | 108 | 9.512 | 31.022 | 10.301 | 1.00 | 48.51 | O |
| ATOM | 9528 | N | GLU | C | 109 | 8.881 | 29.034 | 11.076 | 1.00 | 43.65 | N |
| ATOM | 9530 | CA | GLU | C | 109 | 9.972 | 28.931 | 12.034 | 1.00 | 47.68 | C |
| ATOM | 9532 | CB | GLU | C | 109 | 9.636 | 27.867 | 13.068 | 1.00 | 47.08 | C |
| ATOM | 9535 | CG | GLU | C | 109 | 8.554 | 28.184 | 14.078 | 1.00 | 68.30 | C |
| ATOM | 9538 | CD | GLU | C | 109 | 8.096 | 26.914 | 14.795 | 1.00 | 79.74 | C |
| ATOM | 9539 | OE1 | GLU | C | 109 | 8.789 | 25.877 | 14.668 | 1.00 | 69.75 | O |
| ATOM | 9540 | OE2 | GLU | C | 109 | 7.039 | 26.936 | 15.464 | 1.00 | 67.42 | O |
| ATOM | 9541 | C | GLU | C | 109 | 11.325 | 28.542 | 11.385 | 1.00 | 41.74 | C |
| ATOM | 9542 | O | GLU | C | 109 | 12.389 | 28.808 | 11.938 | 1.00 | 41.16 | O |
| ATOM | 9543 | N | LYS | C | 110 | 11.296 | 27.900 | 10.222 | 1.00 | 39.69 | N |
| ATOM | 9545 | CA | LYS | C | 110 | 12.532 | 27.539 | 9.520 | 1.00 | 34.36 | C |
| ATOM | 9547 | CB | LYS | C | 110 | 12.758 | 26.024 | 9.509 | 1.00 | 28.06 | C |
| ATOM | 9550 | CG | LYS | C | 110 | 13.954 | 25.543 | 8.688 | 1.00 | 38.52 | C |
| ATOM | 9553 | CD | LYS | C | 110 | 13.897 | 24.016 | 8.480 | 1.00 | 31.99 | C |
| ATOM | 9556 | CE | LYS | C | 110 | 15.241 | 23.490 | 8.026 | 1.00 | 38.52 | C |
| ATOM | 9559 | NZ | LYS | C | 110 | 15.264 | 22.033 | 7.774 | 1.00 | 25.09 | N |
| ATOM | 9563 | C | LYS | C | 110 | 12.391 | 28.074 | 8.111 | 1.00 | 37.09 | C |
| ATOM | 9564 | O | LYS | C | 110 | 11.445 | 27.735 | 7.408 | 1.00 | 36.53 | O |
| ATOM | 9565 | N | VAL | C | 111 | 13.362 | 28.898 | 7.716 | 1.00 | 31.59 | N |
| ATOM | 9567 | CA | VAL | C | 111 | 13.473 | 29.431 | 6.371 | 1.00 | 24.86 | C |
| ATOM | 9569 | CB | VAL | C | 111 | 13.228 | 30.945 | 6.457 | 1.00 | 34.11 | C |
| ATOM | 9571 | CG1 | VAL | C | 111 | 13.239 | 31.594 | 5.087 | 1.00 | 32.41 | C |
| ATOM | 9575 | CG2 | VAL | C | 111 | 11.908 | 31.185 | 7.251 | 1.00 | 35.91 | C |
| ATOM | 9579 | C | VAL | C | 111 | 14.883 | 29.187 | 5.815 | 1.00 | 25.58 | C |
| ATOM | 9580 | O | VAL | C | 111 | 15.893 | 29.578 | 6.404 | 1.00 | 42.36 | O |
| ATOM | 9581 | N | SER | C | 112 | 14.937 | 28.594 | 4.638 | 1.00 | 29.23 | N |
| ATOM | 9583 | CA | SER | C | 112 | 16.176 | 28.274 | 3.951 | 1.00 | 30.85 | C |
| ATOM | 9585 | CB | SER | C | 112 | 16.188 | 26.808 | 3.499 | 1.00 | 27.04 | C |
| ATOM | 9588 | OG | SER | C | 112 | 16.140 | 25.944 | 4.624 | 1.00 | 19.85 | O |
| ATOM | 9590 | C | SER | C | 112 | 16.257 | 29.141 | 2.718 | 1.00 | 33.07 | C |
| ATOM | 9591 | O | SER | C | 112 | 15.245 | 29.489 | 2.129 | 1.00 | 40.61 | O |
| ATOM | 9592 | N | ASP | C | 113 | 17.486 | 29.459 | 2.319 | 1.00 | 33.20 | N |
| ATOM | 9594 | CA | ASP | C | 113 | 17.731 | 30.423 | 1.269 | 1.00 | 30.91 | C |
| ATOM | 9596 | CB | ASP | C | 113 | 18.019 | 31.790 | 1.920 | 1.00 | 38.85 | C |
| ATOM | 9599 | CG | ASP | C | 113 | 18.377 | 32.827 | 0.936 | 1.00 | 46.78 | C |
| ATOM | 9600 | OD1 | ASP | C | 113 | 17.520 | 33.697 | 0.678 | 1.00 | 84.73 | O |
| ATOM | 9601 | OD2 | ASP | C | 113 | 19.496 | 32.888 | 0.383 | 1.00 | 82.62 | O |
| ATOM | 9602 | C | ASP | C | 113 | 18.959 | 29.871 | 0.565 | 1.00 | 30.28 | C |
| ATOM | 9603 | O | ASP | C | 113 | 20.070 | 29.887 | 1.117 | 1.00 | 30.93 | O |
| ATOM | 9604 | N | TYR | C | 114 | 18.749 | 29.422 | −0.674 | 1.00 | 31.85 | N |
| ATOM | 9606 | CA | TYR | C | 114 | 19.806 | 28.891 | −1.507 | 1.00 | 31.03 | C |
| ATOM | 9608 | CB | TYR | C | 114 | 19.464 | 27.468 | −1.911 | 1.00 | 28.70 | C |
| ATOM | 9611 | CG | TYR | C | 114 | 19.468 | 26.538 | −0.754 | 1.00 | 28.14 | C |
| ATOM | 9612 | CD1 | TYR | C | 114 | 18.281 | 26.229 | −0.080 | 1.00 | 30.40 | C |
| ATOM | 9614 | CE1 | TYR | C | 114 | 18.272 | 25.299 | 0.976 | 1.00 | 31.68 | C |
| ATOM | 9616 | CZ | TYR | C | 114 | 19.471 | 24.786 | 1.469 | 1.00 | 31.41 | C |
| ATOM | 9617 | OH | TYR | C | 114 | 19.462 | 23.886 | 2.517 | 1.00 | 27.41 | O |
| ATOM | 9619 | CE2 | TYR | C | 114 | 20.660 | 25.069 | 0.810 | 1.00 | 24.80 | C |
| ATOM | 9621 | CD2 | TYR | C | 114 | 20.656 | 25.958 | −0.293 | 1.00 | 24.46 | C |
| ATOM | 9623 | C | TYR | C | 114 | 19.963 | 29.748 | −2.746 | 1.00 | 31.25 | C |
| ATOM | 9624 | O | TYR | C | 114 | 19.039 | 30.437 | −3.155 | 1.00 | 34.12 | O |
| ATOM | 9625 | N | GLU | C | 115 | 21.198 | 29.814 | −3.232 | 1.00 | 26.91 | N |
| ATOM | 9627 | CA | GLU | C | 115 | 21.535 | 30.630 | −4.392 | 1.00 | 24.45 | C |
| ATOM | 9629 | CB | GLU | C | 115 | 22.300 | 31.859 | −3.942 | 1.00 | 22.70 | C |
| ATOM | 9632 | CG | GLU | C | 115 | 22.589 | 32.873 | −5.029 | 1.00 | 43.76 | C |
| ATOM | 9635 | CD | GLU | C | 115 | 23.116 | 34.160 | −4.430 | 1.00 | 69.41 | C |
| ATOM | 9636 | OE1 | GLU | C | 115 | 22.287 | 35.047 | −4.101 | 1.00 | 74.33 | O |
| ATOM | 9637 | OE2 | GLU | C | 115 | 24.352 | 34.237 | −4.230 | 1.00 | 64.61 | O |
| ATOM | 9638 | C | GLU | C | 115 | 22.345 | 29.760 | −5.328 | 1.00 | 30.30 | C |
| ATOM | 9639 | O | GLU | C | 115 | 23.491 | 29.377 | −5.010 | 1.00 | 32.16 | O |
| ATOM | 9640 | N | MET | C | 116 | 21.673 | 29.329 | −6.388 | 1.00 | 23.45 | N |
| ATOM | 9642 | CA | MET | C | 116 | 22.255 | 28.434 | −7.378 | 1.00 | 30.37 | C |
| ATOM | 9644 | CB | MET | C | 116 | 21.162 | 27.702 | −8.113 | 1.00 | 30.72 | C |
| ATOM | 9647 | CG | MET | C | 116 | 21.664 | 26.486 | −8.730 | 1.00 | 33.66 | C |
| ATOM | 9650 | SD | MET | C | 116 | 20.443 | 25.421 | −9.255 | 1.00 | 41.25 | S |
| ATOM | 9651 | CE | MET | C | 116 | 19.353 | 26.547 | −9.937 | 1.00 | 42.94 | C |
| ATOM | 9655 | C | MET | C | 116 | 23.057 | 29.171 | −8.421 | 1.00 | 41.93 | C |
| ATOM | 9656 | O | MET | C | 116 | 22.659 | 30.226 | −8.888 | 1.00 | 49.90 | O |
| ATOM | 9657 | N | LYS | C | 117 | 24.215 | 28.620 | −8.752 | 1.00 | 51.38 | N |
| ATOM | 9659 | CA | LYS | C | 117 | 25.003 | 29.055 | −9.905 | 1.00 | 52.91 | C |
| ATOM | 9661 | CB | LYS | C | 117 | 26.391 | 28.374 | −9.833 | 1.00 | 55.70 | C |
| ATOM | 9664 | CG | LYS | C | 117 | 27.307 | 28.854 | −8.686 | 1.00 | 64.95 | C |
| ATOM | 9667 | CD | LYS | C | 117 | 28.480 | 27.892 | −8.408 | 1.00 | 73.14 | C |
| ATOM | 9670 | CE | LYS | C | 117 | 29.509 | 28.471 | −7.416 | 1.00 | 78.44 | C |
| ATOM | 9673 | NZ | LYS | C | 117 | 30.843 | 27.774 | −7.461 | 1.00 | 65.82 | N |
| ATOM | 9677 | C | LYS | C | 117 | 24.310 | 28.643 | −11.220 | 1.00 | 48.65 | C |
| ATOM | 9678 | O | LYS | C | 117 | 24.097 | 27.461 | −11.472 | 1.00 | 46.54 | O |
| ATOM | 9679 | N | LEU | C | 118 | 24.012 | 29.592 | −12.094 | 1.00 | 45.66 | N |
| ATOM | 9681 | CA | LEU | C | 118 | 23.612 | 29.217 | −13.440 | 1.00 | 51.98 | C |
| ATOM | 9683 | CB | LEU | C | 118 | 22.846 | 30.376 | −14.093 | 1.00 | 52.58 | C |
| ATOM | 9686 | CG | LEU | C | 118 | 21.599 | 30.899 | −13.359 | 1.00 | 55.27 | C |
| ATOM | 9688 | CD1 | LEU | C | 118 | 20.978 | 31.988 | −14.165 | 1.00 | 52.38 | C |
| ATOM | 9692 | CD2 | LEU | C | 118 | 20.545 | 29.820 | −13.079 | 1.00 | 42.40 | C |
| ATOM | 9696 | C | LEU | C | 118 | 24.815 | 28.774 | −14.304 | 1.00 | 61.94 | C |
| ATOM | 9697 | O | LEU | C | 118 | 25.972 | 29.116 | −14.021 | 1.00 | 65.76 | O |
| ATOM | 9698 | N | MET | C | 119 | 24.555 | 27.963 | −15.325 | 1.00 | 61.99 | N |
| ATOM | 9700 | CA | MET | C | 119 | 25.522 | 27.753 | −16.395 | 1.00 | 63.97 | C |
| ATOM | 9702 | CB | MET | C | 119 | 25.921 | 26.284 | −16.542 | 1.00 | 57.19 | C |
| ATOM | 9705 | CG | MET | C | 119 | 24.794 | 25.320 | −16.856 | 1.00 | 62.45 | C |
| ATOM | 9708 | SD | MET | C | 119 | 25.352 | 23.578 | −17.048 | 1.00 | 89.15 | S |
| ATOM | 9709 | CE | MET | C | 119 | 27.186 | 23.752 | −16.939 | 1.00 | 84.58 | C |
| ATOM | 9713 | C | MET | C | 119 | 25.046 | 28.324 | −17.729 | 1.00 | 70.67 | C |
| ATOM | 9714 | O | MET | C | 119 | 23.855 | 28.373 | −18.040 | 1.00 | 71.01 | O |
| ATOM | 9715 | N | ASP | C | 120 | 26.006 | 28.773 | −18.523 | 1.00 | 82.74 | N |
| ATOM | 9717 | CA | ASP | C | 120 | 25.694 | 29.567 | −19.706 | 1.00 | 90.23 | C |
| ATOM | 9719 | CB | ASP | C | 120 | 26.740 | 30.681 | −19.876 | 1.00 | 94.08 | C |
| ATOM | 9722 | CG | ASP | C | 120 | 26.468 | 31.896 | −18.971 | 1.00 | 99.28 | C |
| ATOM | 9723 | OD1 | ASP | C | 120 | 25.283 | 32.248 | −18.766 | 1.00 | 107.16 | O |
| ATOM | 9724 | OD2 | ASP | C | 120 | 27.376 | 32.557 | −18.418 | 1.00 | 95.47 | O |
| ATOM | 9725 | C | ASP | C | 120 | 25.587 | 28.660 | −20.942 | 1.00 | 90.30 | C |
| ATOM | 9726 | O | ASP | C | 120 | 26.532 | 27.937 | −21.275 | 1.00 | 82.45 | O |
| ATOM | 9727 | N | LEU | C | 121 | 24.401 | 28.654 | −21.556 | 1.00 | 91.92 | N |
| ATOM | 9729 | CA | LEU | C | 121 | 24.061 | 27.698 | −22.608 | 1.00 | 93.84 | C |
| ATOM | 9731 | CB | LEU | C | 121 | 23.259 | 26.503 | −22.059 | 1.00 | 90.75 | C |
| ATOM | 9734 | CG | LEU | C | 121 | 23.395 | 25.996 | −20.617 | 1.00 | 86.89 | C |
| ATOM | 9736 | CD1 | LEU | C | 121 | 22.029 | 25.528 | −20.109 | 1.00 | 85.71 | C |
| ATOM | 9740 | CD2 | LEU | C | 121 | 24.419 | 24.866 | −20.479 | 1.00 | 77.22 | C |
| ATOM | 9744 | C | LEU | C | 121 | 23.278 | 28.343 | −23.763 | 1.00 | 97.91 | C |
| ATOM | 9745 | O | LEU | C | 121 | 22.528 | 29.309 | −23.574 | 1.00 | 95.72 | O |
| ATOM | 9746 | N | ASP | C | 122 | 23.463 | 27.769 | −24.953 | 1.00 | 104.43 | N |
| ATOM | 9748 | CA | ASP | C | 122 | 22.720 | 28.134 | −26.162 | 1.00 | 109.20 | C |
| ATOM | 9750 | CB | ASP | C | 122 | 23.544 | 27.802 | −27.419 | 1.00 | 109.51 | C |
| ATOM | 9753 | CG | ASP | C | 122 | 24.788 | 28.673 | −27.560 | 1.00 | 105.65 | C |
| ATOM | 9754 | OD1 | ASP | C | 122 | 25.905 | 28.122 | −27.682 | 1.00 | 80.40 | O |
| ATOM | 9755 | OD2 | ASP | C | 122 | 24.744 | 29.921 | −27.567 | 1.00 | 105.52 | O |
| ATOM | 9756 | C | ASP | C | 122 | 21.379 | 27.393 | −26.227 | 1.00 | 112.69 | C |
| ATOM | 9757 | O | ASP | C | 122 | 21.344 | 26.161 | −26.301 | 1.00 | 112.65 | O |
| ATOM | 9758 | N | VAL | C | 123 | 20.286 | 28.156 | −26.224 | 1.00 | 116.42 | N |
| ATOM | 9760 | CA | VAL | C | 123 | 18.933 | 27.597 | −26.201 | 1.00 | 118.58 | C |
| ATOM | 9762 | CB | VAL | C | 123 | 17.851 | 28.719 | −26.197 | 1.00 | 118.00 | C |
| ATOM | 9764 | CG1 | VAL | C | 123 | 16.503 | 28.176 | −25.738 | 1.00 | 118.37 | C |
| ATOM | 9768 | CG2 | VAL | C | 123 | 18.273 | 29.889 | −25.314 | 1.00 | 116.77 | C |
| ATOM | 9772 | C | VAL | C | 123 | 18.695 | 26.664 | −27.397 | 1.00 | 120.24 | C |
| ATOM | 9773 | O | VAL | C | 123 | 19.276 | 26.841 | −28.471 | 1.00 | 120.62 | O |
| ATOM | 9774 | N | GLU | C | 124 | 17.865 | 25.647 | −27.186 | 1.00 | 121.30 | N |
| ATOM | 9776 | CA | GLU | C | 124 | 17.331 | 24.844 | −28.279 | 1.00 | 121.01 | C |
| ATOM | 9778 | CB | GLU | C | 124 | 17.325 | 23.358 | −27.897 | 1.00 | 121.85 | C |
| ATOM | 9781 | CG | GLU | C | 124 | 18.678 | 22.794 | −27.481 | 1.00 | 119.99 | C |
| ATOM | 9784 | CD | GLU | C | 124 | 18.708 | 21.274 | −27.479 | 1.00 | 117.49 | C |
| ATOM | 9785 | OE1 | GLU | C | 124 | 19.079 | 20.683 | −28.515 | 1.00 | 120.63 | O |
| ATOM | 9786 | OE2 | GLU | C | 124 | 18.373 | 20.660 | −26.442 | 1.00 | 113.84 | O |
| ATOM | 9787 | C | GLU | C | 124 | 15.907 | 25.312 | −28.587 | 1.00 | 120.98 | C |
| ATOM | 9788 | O | GLU | C | 124 | 15.259 | 25.946 | −27.752 | 1.00 | 120.50 | O |
| ATOM | 9789 | N | GLN | C | 125 | 15.422 | 24.997 | −29.785 | 1.00 | 121.08 | N |
| ATOM | 9791 | CA | GLN | C | 125 | 14.080 | 25.397 | −30.196 | 1.00 | 119.54 | C |
| ATOM | 9793 | CB | GLN | C | 125 | 14.152 | 26.776 | −30.866 | 1.00 | 119.69 | C |
| ATOM | 9796 | CG | GLN | C | 125 | 12.961 | 27.683 | −30.591 | 1.00 | 118.04 | C |
| ATOM | 9799 | CD | GLN | C | 125 | 11.897 | 27.591 | −31.676 | 1.00 | 121.74 | C |
| ATOM | 9800 | OE1 | GLN | C | 125 | 12.197 | 27.725 | −32.867 | 1.00 | 121.52 | O |
| ATOM | 9801 | NE2 | GLN | C | 125 | 10.657 | 27.342 | −31.269 | 1.00 | 112.72 | N |
| ATOM | 9804 | C | GLN | C | 125 | 13.458 | 24.363 | −31.150 | 1.00 | 118.39 | C |
| ATOM | 9805 | O | GLN | C | 125 | 14.074 | 23.996 | −32.153 | 1.00 | 119.49 | O |
| ATOM | 9806 | N | LEU | C | 126 | 12.269 | 23.863 | −30.809 | 1.00 | 116.30 | N |
| ATOM | 9808 | CA | LEU | C | 126 | 11.391 | 23.178 | −31.769 | 1.00 | 114.58 | C |
| ATOM | 9810 | CB | LEU | C | 126 | 11.831 | 21.724 | −31.999 | 1.00 | 111.61 | C |
| ATOM | 9813 | CG | LEU | C | 126 | 10.861 | 20.808 | −32.764 | 1.00 | 105.85 | C |
| ATOM | 9815 | CD1 | LEU | C | 126 | 10.664 | 21.232 | −34.213 | 1.00 | 104.82 | C |
| ATOM | 9819 | CD2 | LEU | C | 126 | 11.313 | 19.361 | −32.708 | 1.00 | 96.62 | C |
| ATOM | 9823 | C | LEU | C | 126 | 9.900 | 23.247 | −31.378 | 1.00 | 115.54 | C |
| ATOM | 9824 | O | LEU | C | 126 | 9.142 | 24.045 | −31.940 | 1.00 | 114.99 | O |
| ATOM | 9825 | N | GLY | C | 127 | 9.476 | 22.403 | −30.437 | 1.00 | 115.60 | N |
| ATOM | 9827 | CA | GLY | C | 127 | 8.086 | 22.356 | −30.004 | 1.00 | 113.74 | C |
| ATOM | 9830 | C | GLY | C | 127 | 7.279 | 21.252 | −30.670 | 1.00 | 113.75 | C |
| ATOM | 9831 | O | GLY | C | 127 | 6.227 | 20.858 | −30.163 | 1.00 | 110.61 | O |
| ATOM | 9832 | N | ILE | C | 128 | 7.764 | 20.791 | −31.825 | 1.00 | 113.67 | N |
| ATOM | 9834 | CA | ILE | C | 128 | 7.197 | 19.665 | −32.570 | 1.00 | 111.30 | C |
| ATOM | 9836 | CB | ILE | C | 128 | 7.179 | 18.376 | −31.726 | 1.00 | 111.57 | C |
| ATOM | 9838 | CG1 | ILE | C | 128 | 7.935 | 17.253 | −32.443 | 1.00 | 106.79 | C |
| ATOM | 9841 | CD1 | ILE | C | 128 | 7.047 | 16.111 | −32.935 | 1.00 | 106.56 | C |
| ATOM | 9845 | CG2 | ILE | C | 128 | 5.740 | 17.940 | −31.457 | 1.00 | 113.07 | C |
| ATOM | 9849 | C | ILE | C | 128 | 5.800 | 19.930 | −33.120 | 1.00 | 112.65 | C |
| ATOM | 9850 | O | ILE | C | 128 | 5.037 | 20.735 | −32.572 | 1.00 | 109.14 | O |
| ATOM | 9851 | N | PRO | C | 129 | 5.476 | 19.221 | −34.201 | 1.00 | 113.55 | N |
| ATOM | 9852 | CA | PRO | C | 129 | 4.194 | 19.374 | −34.897 | 1.00 | 112.20 | C |
| ATOM | 9854 | CB | PRO | C | 129 | 4.398 | 18.569 | −36.184 | 1.00 | 112.66 | C |
| ATOM | 9857 | CG | PRO | C | 129 | 5.435 | 17.552 | −35.837 | 1.00 | 115.20 | C |
| ATOM | 9860 | CD | PRO | C | 129 | 6.344 | 18.231 | −34.862 | 1.00 | 113.78 | C |
| ATOM | 9863 | C | PRO | C | 129 | 3.010 | 18.824 | −34.110 | 1.00 | 110.58 | C |
| ATOM | 9864 | O | PRO | C | 129 | 2.883 | 17.604 | −33.947 | 1.00 | 108.56 | O |
| ATOM | 9865 | N | GLU | C | 130 | 2.167 | 19.743 | −33.638 | 1.00 | 106.91 | N |
| ATOM | 9867 | CA | GLU | C | 130 | 0.915 | 19.425 | −32.956 | 1.00 | 102.92 | C |
| ATOM | 9869 | CB | GLU | C | 130 | 0.243 | 20.718 | −32.481 | 1.00 | 104.57 | C |
| ATOM | 9872 | CG | GLU | C | 130 | −0.784 | 20.519 | −31.378 | 1.00 | 108.12 | C |
| ATOM | 9875 | CD | GLU | C | 130 | −1.710 | 21.711 | −31.230 | 1.00 | 112.79 | C |
| ATOM | 9876 | OE1 | GLU | C | 130 | −2.913 | 21.573 | −31.546 | 1.00 | 109.22 | O |
| ATOM | 9877 | OE2 | GLU | C | 130 | −1.232 | 22.786 | −30.806 | 1.00 | 117.48 | O |
| ATOM | 9878 | C | GLU | C | 130 | −0.019 | 18.680 | −33.899 | 1.00 | 96.65 | C |
| ATOM | 9879 | O | GLU | C | 130 | −0.210 | 19.093 | −35.039 | 1.00 | 98.42 | O |
| ATOM | 9880 | N | GLN | C | 131 | −0.628 | 17.605 | −33.411 | 1.00 | 90.55 | N |
| ATOM | 9882 | CA | GLN | C | 131 | −1.109 | 16.546 | −34.287 | 1.00 | 82.59 | C |
| ATOM | 9884 | CB | GLN | C | 131 | −0.058 | 15.446 | −34.350 | 1.00 | 80.76 | C |
| ATOM | 9887 | CG | GLN | C | 131 | 0.670 | 15.365 | −35.656 | 1.00 | 83.47 | C |
| ATOM | 9890 | CD | GLN | C | 131 | 1.135 | 13.958 | −35.947 | 1.00 | 91.93 | C |
| ATOM | 9891 | OE1 | GLN | C | 131 | 0.316 | 13.036 | −36.039 | 1.00 | 89.71 | O |
| ATOM | 9892 | NE2 | GLN | C | 131 | 2.450 | 13.783 | −36.079 | 1.00 | 89.98 | N |
| ATOM | 9895 | C | GLN | C | 131 | −2.415 | 15.935 | −33.806 | 1.00 | 77.49 | C |
| ATOM | 9896 | O | GLN | C | 131 | −2.738 | 15.978 | −32.626 | 1.00 | 82.82 | O |
| ATOM | 9897 | N | GLU | C | 132 | −3.153 | 15.327 | −34.721 | 1.00 | 76.06 | N |
| ATOM | 9899 | CA | GLU | C | 132 | −4.289 | 14.500 | −34.335 | 1.00 | 79.13 | C |
| ATOM | 9901 | CB | GLU | C | 132 | −5.489 | 14.724 | −35.279 | 1.00 | 79.77 | C |
| ATOM | 9904 | CG | GLU | C | 132 | −6.712 | 13.867 | −34.958 | 1.00 | 82.66 | C |
| ATOM | 9907 | CD | GLU | C | 132 | −7.933 | 14.239 | −35.783 | 1.00 | 95.13 | C |
| ATOM | 9908 | OE1 | GLU | C | 132 | −7.765 | 14.957 | −36.801 | 1.00 | 82.74 | O |
| ATOM | 9909 | OE2 | GLU | C | 132 | −9.057 | 13.810 | −35.415 | 1.00 | 94.63 | O |
| ATOM | 9910 | C | GLU | C | 132 | −3.806 | 13.056 | −34.404 | 1.00 | 74.35 | C |
| ATOM | 9911 | O | GLU | C | 132 | −3.330 | 12.611 | −35.447 | 1.00 | 77.41 | O |
| ATOM | 9912 | N | TYR | C | 133 | −3.923 | 12.320 | −33.303 | 1.00 | 65.90 | N |
| ATOM | 9914 | CA | TYR | C | 133 | −3.273 | 11.020 | −33.233 | 1.00 | 53.91 | C |
| ATOM | 9916 | CB | TYR | C | 133 | −2.579 | 10.837 | −31.882 | 1.00 | 56.82 | C |
| ATOM | 9919 | CG | TYR | C | 133 | −1.439 | 11.806 | −31.630 | 1.00 | 37.17 | C |
| ATOM | 9920 | CD1 | TYR | C | 133 | −1.668 | 13.041 | −31.021 | 1.00 | 36.51 | C |
| ATOM | 9922 | CE1 | TYR | C | 133 | −0.619 | 13.925 | −30.753 | 1.00 | 43.60 | C |
| ATOM | 9924 | CZ | TYR | C | 133 | 0.672 | 13.598 | −31.185 | 1.00 | 46.17 | C |
| ATOM | 9925 | OH | TYR | C | 133 | 1.720 | 14.460 | −30.971 | 1.00 | 40.63 | O |
| ATOM | 9927 | CE2 | TYR | C | 133 | 0.919 | 12.384 | −31.806 | 1.00 | 39.54 | C |
| ATOM | 9929 | CD2 | TYR | C | 133 | −0.131 | 11.498 | −32.034 | 1.00 | 45.57 | C |
| ATOM | 9931 | C | TYR | C | 133 | −4.309 | 9.935 | −33.480 | 1.00 | 50.89 | C |
| ATOM | 9932 | O | TYR | C | 133 | −5.489 | 10.151 | −33.251 | 1.00 | 54.96 | O |
| ATOM | 9933 | N | SER | C | 134 | −3.855 | 8.796 | −33.990 | 1.00 | 41.98 | N |
| ATOM | 9935 | CA | SER | C | 134 | −4.674 | 7.616 | −34.232 | 1.00 | 50.16 | C |
| ATOM | 9937 | CB | SER | C | 134 | −3.850 | 6.538 | −34.957 | 1.00 | 52.09 | C |
| ATOM | 9940 | OG | SER | C | 134 | −3.211 | 7.047 | −36.109 | 1.00 | 53.93 | O |
| ATOM | 9942 | C | SER | C | 134 | −5.247 | 6.961 | −32.966 | 1.00 | 50.58 | C |
| ATOM | 9943 | O | SER | C | 134 | −6.408 | 6.540 | −32.997 | 1.00 | 44.43 | O |
| ATOM | 9944 | N | CYS | C | 135 | −4.384 | 6.734 | −31.966 | 1.00 | 44.11 | N |
| ATOM | 9946 | CA | CYS | C | 135 | −4.752 | 6.175 | −30.661 | 1.00 | 51.78 | C |
| ATOM | 9948 | CB | CYS | C | 135 | −4.059 | 4.836 | −30.379 | 1.00 | 38.05 | C |
| ATOM | 9951 | SG | CYS | C | 135 | −4.649 | 3.546 | −31.455 | 1.00 | 84.46 | S |
| ATOM | 9952 | C | CYS | C | 135 | −4.357 | 7.121 | −29.550 | 1.00 | 42.21 | C |
| ATOM | 9953 | O | CYS | C | 135 | −3.281 | 7.704 | −29.608 | 1.00 | 37.73 | O |
| ATOM | 9954 | N | VAL | C | 136 | −5.226 | 7.204 | −28.545 | 1.00 | 36.40 | N |
| ATOM | 9956 | CA | VAL | C | 136 | −4.962 | 7.836 | −27.257 | 1.00 | 39.36 | C |
| ATOM | 9958 | CB | VAL | C | 136 | −5.767 | 9.118 | −27.064 | 1.00 | 35.96 | C |
| ATOM | 9960 | CG1 | VAL | C | 136 | −5.378 | 9.794 | −25.738 | 1.00 | 24.15 | C |
| ATOM | 9964 | CG2 | VAL | C | 136 | −5.540 | 10.065 | −28.241 | 1.00 | 50.64 | C |
| ATOM | 9968 | C | VAL | C | 136 | −5.407 | 6.873 | −26.166 | 1.00 | 37.77 | C |
| ATOM | 9969 | O | VAL | C | 136 | −6.603 | 6.593 | −26.043 | 1.00 | 46.23 | O |
| ATOM | 9970 | N | VAL | C | 137 | −4.434 | 6.347 | −25.427 | 1.00 | 34.78 | N |
| ATOM | 9972 | CA | VAL | C | 137 | −4.665 | 5.480 | −24.282 | 1.00 | 32.96 | C |
| ATOM | 9974 | CB | VAL | C | 137 | −3.606 | 4.375 | −24.274 | 1.00 | 38.59 | C |
| ATOM | 9976 | CG1 | VAL | C | 137 | −3.805 | 3.449 | −23.066 | 1.00 | 43.22 | C |
| ATOM | 9980 | CG2 | VAL | C | 137 | −3.599 | 3.639 | −25.599 | 1.00 | 29.46 | C |
| ATOM | 9984 | C | VAL | C | 137 | −4.506 | 6.306 | −23.019 | 1.00 | 32.26 | C |
| ATOM | 9985 | O | VAL | C | 137 | −3.456 | 6.948 | −22.806 | 1.00 | 35.74 | O |
| ATOM | 9986 | N | LYS | C | 138 | −5.563 | 6.340 | −22.211 | 1.00 | 37.18 | N |
| ATOM | 9988 | CA | LYS | C | 138 | −5.467 | 6.840 | −20.845 | 1.00 | 34.58 | C |
| ATOM | 9990 | CB | LYS | C | 138 | −6.599 | 7.810 | −20.567 | 1.00 | 43.75 | C |
| ATOM | 9993 | CG | LYS | C | 138 | −6.590 | 8.371 | −19.152 | 1.00 | 54.29 | C |
| ATOM | 9996 | CD | LYS | C | 138 | −7.362 | 9.690 | −19.098 | 1.00 | 67.82 | C |
| ATOM | 9999 | CE | LYS | C | 138 | −7.591 | 10.150 | −17.651 | 1.00 | 67.72 | C |
| ATOM | 10002 | NZ | LYS | C | 138 | −8.238 | 9.072 | −16.847 | 1.00 | 48.83 | N |
| ATOM | 10006 | C | LYS | C | 138 | −5.461 | 5.698 | −19.832 | 1.00 | 31.34 | C |
| ATOM | 10007 | O | LYS | C | 138 | −6.246 | 4.778 | −19.955 | 1.00 | 32.35 | O |
| ATOM | 10008 | N | MET | C | 139 | −4.530 | 5.720 | −18.878 | 1.00 | 28.70 | N |
| ATOM | 10010 | CA | MET | C | 139 | −4.359 | 4.606 | −17.958 | 1.00 | 29.48 | C |
| ATOM | 10012 | CB | MET | C | 139 | −3.547 | 3.492 | −18.638 | 1.00 | 35.64 | C |
| ATOM | 10015 | CG | MET | C | 139 | −2.033 | 3.623 | −18.527 | 1.00 | 44.11 | C |
| ATOM | 10018 | SD | MET | C | 139 | −1.106 | 2.234 | −19.391 | 1.00 | 36.71 | S |
| ATOM | 10019 | CE | MET | C | 139 | −1.135 | 3.047 | −20.763 | 1.00 | 24.62 | C |
| ATOM | 10023 | C | MET | C | 139 | −3.670 | 5.034 | −16.680 | 1.00 | 26.91 | C |
| ATOM | 10024 | O | MET | C | 139 | −3.085 | 6.109 | −16.646 | 1.00 | 26.60 | O |
| ATOM | 10025 | N | PRO | C | 140 | −3.736 | 4.234 | −15.614 | 1.00 | 29.57 | N |
| ATOM | 10026 | CA | PRO | C | 140 | −3.046 | 4.602 | −14.363 | 1.00 | 25.40 | C |
| ATOM | 10028 | CB | PRO | C | 140 | −3.331 | 3.400 | −13.430 | 1.00 | 21.40 | C |
| ATOM | 10031 | CG | PRO | C | 140 | −4.582 | 2.800 | −13.953 | 1.00 | 32.12 | C |
| ATOM | 10034 | CD | PRO | C | 140 | −4.491 | 2.970 | −15.467 | 1.00 | 34.75 | C |
| ATOM | 10037 | C | PRO | C | 140 | −1.542 | 4.746 | −14.623 | 1.00 | 31.73 | C |
| ATOM | 10038 | O | PRO | C | 140 | −0.932 | 3.924 | −15.318 | 1.00 | 34.78 | O |
| ATOM | 10039 | N | SER | C | 141 | −0.943 | 5.721 | −13.953 | 1.00 | 38.36 | N |
| ATOM | 10041 | CA | SER | C | 141 | 0.455 | 6.018 | −14.083 | 1.00 | 31.38 | C |
| ATOM | 10043 | CB | SER | C | 141 | 0.747 | 7.442 | −13.556 | 1.00 | 31.43 | C |
| ATOM | 10046 | OG | SER | C | 141 | 0.776 | 7.560 | −12.144 | 1.00 | 30.72 | O |
| ATOM | 10048 | C | SER | C | 141 | 1.287 | 4.887 | −13.447 | 1.00 | 41.09 | C |
| ATOM | 10049 | O | SER | C | 141 | 2.283 | 4.436 | −14.043 | 1.00 | 27.89 | O |
| ATOM | 10050 | N | GLY | C | 142 | 0.827 | 4.343 | −12.312 | 1.00 | 34.09 | N |
| ATOM | 10052 | CA | GLY | C | 142 | 1.519 | 3.218 | −11.679 | 1.00 | 30.57 | C |
| ATOM | 10055 | C | GLY | C | 142 | 1.513 | 1.946 | −12.532 | 1.00 | 34.31 | C |
| ATOM | 10056 | O | GLY | C | 142 | 2.466 | 1.144 | −12.571 | 1.00 | 32.55 | O |
| ATOM | 10057 | N | GLY | C | 143 | 0.419 | 1.749 | −13.259 | 1.00 | 25.96 | N |
| ATOM | 10059 | CA | GLU | C | 143 | 0.381 | 0.607 | −14.177 | 1.00 | 32.56 | C |
| ATOM | 10061 | CB | GLU | C | 143 | −1.009 | 0.350 | −14.700 | 1.00 | 28.16 | C |
| ATOM | 10064 | CG | GLU | C | 143 | −1.194 | −1.082 | −15.195 | 1.00 | 43.75 | C |
| ATOM | 10067 | CD | GLU | C | 143 | −0.799 | −2.118 | −14.160 | 1.00 | 58.38 | C |
| ATOM | 10068 | OE1 | GLU | C | 143 | −1.542 | −2.292 | −13.161 | 1.00 | 63.09 | O |
| ATOM | 10069 | OE2 | GLU | C | 143 | 0.268 | −2.753 | −14.354 | 1.00 | 53.77 | O |
| ATOM | 10070 | C | GLU | C | 143 | 1.296 | 0.716 | −15.389 | 1.00 | 30.32 | C |
| ATOM | 10071 | O | GLU | C | 143 | 1.873 | −0.278 | −15.805 | 1.00 | 37.40 | O |
| ATOM | 10072 | N | PHE | C | 144 | 1.471 | 1.928 | −15.915 | 1.00 | 28.90 | N |
| ATOM | 10074 | CA | PHE | C | 144 | 2.333 | 2.114 | −17.053 | 1.00 | 29.58 | C |
| ATOM | 10076 | CB | PHE | C | 144 | 2.089 | 3.480 | −17.680 | 1.00 | 31.79 | C |
| ATOM | 10079 | CG | PHE | C | 144 | 2.957 | 3.751 | −18.873 | 1.00 | 30.90 | C |
| ATOM | 10080 | CD1 | PHE | C | 144 | 2.956 | 2.888 | −19.960 | 1.00 | 34.45 | C |
| ATOM | 10082 | CE1 | PHE | C | 144 | 3.791 | 3.118 | −21.045 | 1.00 | 37.47 | C |
| ATOM | 10084 | CZ | PHE | C | 144 | 4.581 | 4.262 | −21.062 | 1.00 | 28.55 | C |
| ATOM | 10086 | CE2 | PHE | C | 144 | 4.581 | 5.127 | −19.961 | 1.00 | 30.24 | C |
| ATOM | 10088 | CD2 | PHE | C | 144 | 3.790 | 4.860 | −18.892 | 1.00 | 28.14 | C |
| ATOM | 10090 | C | PHE | C | 144 | 3.775 | 1.917 | −16.608 | 1.00 | 26.89 | C |
| ATOM | 10091 | O | PHE | C | 144 | 4.535 | 1.232 | −17.305 | 1.00 | 28.32 | O |
| ATOM | 10092 | N | ALA | C | 145 | 4.151 | 2.493 | −15.458 | 1.00 | 20.98 | N |
| ATOM | 10094 | CA | ALA | C | 145 | 5.515 | 2.320 | −14.948 | 1.00 | 25.44 | C |
| ATOM | 10096 | CB | ALA | C | 145 | 5.742 | 3.023 | −13.624 | 1.00 | 24.90 | C |
| ATOM | 10100 | C | ALA | C | 145 | 5.910 | 0.868 | −14.773 | 1.00 | 31.33 | C |
| ATOM | 10101 | O | ALA | C | 145 | 7.052 | 0.510 | −15.031 | 1.00 | 33.14 | O |
| ATOM | 10102 | N | ARG | C | 146 | 4.998 | 0.080 | −14.214 | 1.00 | 34.60 | N |
| ATOM | 10104 | CA | ARG | C | 146 | 5.269 | −1.323 | −13.913 | 1.00 | 34.80 | C |
| ATOM | 10106 | CB | ARG | C | 146 | 4.170 | −1.948 | −13.050 | 1.00 | 30.12 | C |
| ATOM | 10109 | CG | ARG | C | 146 | 4.090 | −3.457 | −13.164 | 1.00 | 40.06 | C |
| ATOM | 10112 | CD | ARG | C | 146 | 3.859 | −4.196 | −11.843 | 1.00 | 74.70 | C |
| ATOM | 10115 | NE | ARG | C | 146 | 5.021 | −4.977 | −11.392 | 1.00 | 91.43 | N |
| ATOM | 10117 | CZ | ARG | C | 146 | 5.132 | −5.535 | −10.182 | 1.00 | 95.89 | C |
| ATOM | 10118 | NH1 | ARG | C | 146 | 4.152 | −5.425 | −9.288 | 1.00 | 89.32 | N |
| ATOM | 10121 | NH2 | ARG | C | 146 | 6.231 | −6.201 | −9.851 | 1.00 | 85.00 | N |
| ATOM | 10124 | C | ARG | C | 146 | 5.437 | −2.141 | −15.180 | 1.00 | 30.79 | C |
| ATOM | 10125 | O | ARG | C | 146 | 6.262 | −3.034 | −15.196 | 1.00 | 32.39 | O |
| ATOM | 10126 | N | ILE | C | 147 | 4.591 | −1.898 | −16.183 | 1.00 | 31.28 | N |
| ATOM | 10128 | CA | ILE | C | 147 | 4.730 | −2.536 | −17.474 | 1.00 | 30.08 | C |
| ATOM | 10130 | CB | ILE | C | 147 | 3.592 | −2.086 | −18.379 | 1.00 | 25.42 | C |
| ATOM | 10132 | CG1 | ILE | C | 147 | 2.305 | −2.726 | −17.880 | 1.00 | 25.92 | C |
| ATOM | 10135 | CD1 | ILE | C | 147 | 1.048 | −2.101 | −18.411 | 1.00 | 30.23 | C |
| ATOM | 10139 | CG2 | ILE | C | 147 | 3.844 | −2.406 | −19.850 | 1.00 | 29.40 | C |
| ATOM | 10143 | C | ILE | C | 147 | 6.099 | −2.296 | −18.112 | 1.00 | 33.16 | C |
| ATOM | 10144 | O | ILE | C | 147 | 6.711 | −3.250 | −18.567 | 1.00 | 37.72 | O |
| ATOM | 10145 | N | CYS | C | 148 | 6.545 | −1.038 | −18.196 | 1.00 | 24.49 | N |
| ATOM | 10147 | CA | CYS | C | 148 | 7.797 | −0.722 | −18.874 | 1.00 | 28.70 | C |
| ATOM | 10149 | CB | CYS | C | 148 | 8.072 | 0.791 | −18.975 | 1.00 | 29.65 | C |
| ATOM | 10152 | SG | CYS | C | 148 | 6.880 | 1.646 | −20.020 | 1.00 | 33.76 | S |
| ATOM | 10153 | C | CYS | C | 148 | 8.912 | −1.377 | −18.135 | 1.00 | 32.76 | C |
| ATOM | 10154 | O | CYS | C | 148 | 9.884 | −1.806 | −18.761 | 1.00 | 32.97 | O |
| ATOM | 10155 | N | ARG | C | 149 | 8.765 | −1.480 | −16.815 | 1.00 | 36.43 | N |
| ATOM | 10157 | CA | ARG | C | 149 | 9.823 | −2.072 | −15.987 | 1.00 | 38.19 | C |
| ATOM | 10159 | CB | ARG | C | 149 | 9.581 | −1.714 | −14.528 | 1.00 | 40.41 | C |
| ATOM | 10162 | CG | ARG | C | 149 | 10.049 | −2.765 | −13.487 | 1.00 | 54.93 | C |
| ATOM | 10165 | CD | ARG | C | 149 | 9.482 | −2.517 | −12.091 | 1.00 | 59.07 | C |
| ATOM | 10168 | NE | ARG | C | 149 | 9.094 | −1.106 | −11.989 | 1.00 | 79.93 | N |
| ATOM | 10170 | CZ | ARG | C | 149 | 8.120 | −0.616 | −11.226 | 1.00 | 69.96 | C |
| ATOM | 10171 | NH1 | ARG | C | 149 | 7.426 | −1.412 | −10.421 | 1.00 | 82.70 | N |
| ATOM | 10174 | NH2 | ARG | C | 149 | 7.885 | 0.695 | −11.235 | 1.00 | 54.66 | N |
| ATOM | 10177 | C | ARG | C | 149 | 9.887 | −3.578 | −16.159 | 1.00 | 35.66 | C |
| ATOM | 10178 | O | ARG | C | 149 | 10.954 | −4.200 | −16.282 | 1.00 | 33.00 | O |
| ATOM | 10179 | N | ASP | C | 150 | 8.710 | −4.194 | −16.164 | 1.00 | 37.79 | N |
| ATOM | 10181 | CA | ASP | C | 150 | 8.657 | −5.633 | −16.340 | 1.00 | 36.28 | C |
| ATOM | 10183 | CB | ASP | C | 150 | 7.233 | −6.097 | −16.122 | 1.00 | 38.34 | C |
| ATOM | 10186 | CG | ASP | C | 150 | 6.822 | −5.960 | −14.662 | 1.00 | 44.97 | C |
| ATOM | 10187 | OD1 | ASP | C | 150 | 7.669 | −5.500 | −13.867 | 1.00 | 38.34 | O |
| ATOM | 10188 | OD2 | ASP | C | 150 | 5.710 | −6.282 | −14.204 | 1.00 | 38.84 | O |
| ATOM | 10189 | C | ASP | C | 150 | 9.266 | −6.075 | −17.685 | 1.00 | 35.95 | C |
| ATOM | 10190 | O | ASP | C | 150 | 10.140 | −6.945 | −17.747 | 1.00 | 40.84 | O |
| ATOM | 10191 | N | LEU | C | 151 | 8.913 | −5.379 | −18.750 | 1.00 | 29.67 | N |
| ATOM | 10193 | CA | LEU | C | 151 | 9.266 | −5.822 | −20.086 | 1.00 | 31.40 | C |
| ATOM | 10195 | CB | LEU | C | 151 | 8.355 | −5.143 | −21.096 | 1.00 | 25.59 | C |
| ATOM | 10198 | CG | LEU | C | 151 | 6.962 | −5.746 | −21.122 | 1.00 | 24.64 | C |
| ATOM | 10200 | CD1 | LEU | C | 151 | 5.992 | −4.974 | −21.989 | 1.00 | 28.10 | C |
| ATOM | 10204 | CD2 | LEU | C | 151 | 7.092 | −7.105 | −21.647 | 1.00 | 36.97 | C |
| ATOM | 10208 | C | LEU | C | 151 | 10.727 | −5.524 | −20.406 | 1.00 | 29.14 | C |
| ATOM | 10209 | O | LEU | C | 151 | 11.239 | −5.993 | −21.406 | 1.00 | 43.66 | O |
| ATOM | 10210 | N | SER | C | 152 | 11.398 | −4.804 | −19.516 | 1.00 | 37.67 | N |
| ATOM | 10212 | CA | SER | C | 152 | 12.737 | −4.334 | −19.758 | 1.00 | 38.02 | C |
| ATOM | 10214 | CB | SER | C | 152 | 12.973 | −2.990 | −19.062 | 1.00 | 35.85 | C |
| ATOM | 10217 | OG | SER | C | 152 | 13.057 | −3.115 | −17.654 | 1.00 | 51.42 | O |
| ATOM | 10219 | C | SER | C | 152 | 13.736 | −5.419 | −19.365 | 1.00 | 40.55 | C |
| ATOM | 10220 | O | SER | C | 152 | 14.867 | −5.450 | −19.850 | 1.00 | 39.58 | O |
| ATOM | 10221 | N | HIS | C | 153 | 13.255 | −6.383 | −18.592 | 1.00 | 49.53 | N |
| ATOM | 10223 | CA | HIS | C | 153 | 13.972 | −7.635 | −18.357 | 1.00 | 50.69 | C |
| ATOM | 10225 | CB | HIS | C | 153 | 13.276 | −8.401 | −17.248 | 1.00 | 47.04 | C |
| ATOM | 10228 | CG | HIS | C | 153 | 13.302 | −7.678 | −15.943 | 1.00 | 43.77 | C |
| ATOM | 10229 | ND1 | HIS | C | 153 | 14.463 | −7.175 | −15.402 | 1.00 | 64.68 | N |
| ATOM | 10231 | CE1 | HIS | C | 153 | 14.191 | −6.592 | −14.250 | 1.00 | 69.88 | C |
| ATOM | 10233 | NE2 | HIS | C | 153 | 12.895 | −6.705 | −14.023 | 1.00 | 58.02 | N |
| ATOM | 10235 | CD2 | HIS | C | 153 | 12.319 | −7.399 | −15.058 | 1.00 | 41.83 | C |
| ATOM | 10237 | C | HIS | C | 153 | 14.053 | −8.535 | −19.581 | 1.00 | 50.69 | C |
| ATOM | 10238 | O | HIS | C | 153 | 15.062 | −9.205 | −19.796 | 1.00 | 56.73 | O |
| ATOM | 10239 | N | ILE | C | 154 | 13.002 | −8.517 | −20.394 | 1.00 | 49.83 | N |
| ATOM | 10241 | CA | ILE | C | 154 | 12.937 | −9.344 | −21.591 | 1.00 | 51.00 | C |
| ATOM | 10243 | CB | ILE | C | 154 | 11.485 | −9.635 | −21.928 | 1.00 | 50.67 | C |
| ATOM | 10245 | CG1 | ILE | C | 154 | 10.782 | −10.212 | −20.708 | 1.00 | 56.79 | C |
| ATOM | 10248 | CD1 | ILE | C | 154 | 11.084 | −11.690 | −20.533 | 1.00 | 69.83 | C |
| ATOM | 10252 | CG2 | ILE | C | 154 | 11.449 | −10.635 | −23.066 | 1.00 | 49.15 | C |
| ATOM | 10256 | C | ILE | C | 154 | 13.590 | −8.748 | −22.845 | 1.00 | 48.68 | C |
| ATOM | 10257 | O | ILE | C | 154 | 14.236 | −9.453 | −23.618 | 1.00 | 52.73 | O |
| ATOM | 10258 | N | GLY | C | 155 | 13.406 | −7.452 | −23.057 | 1.00 | 44.11 | N |
| ATOM | 10260 | CA | GLY | C | 155 | 13.700 | −6.838 | −24.340 | 1.00 | 37.89 | C |
| ATOM | 10263 | C | GLY | C | 155 | 14.178 | −5.417 | −24.176 | 1.00 | 39.09 | C |
| ATOM | 10264 | O | GLY | C | 155 | 13.982 | −4.810 | −23.124 | 1.00 | 40.42 | O |
| ATOM | 10265 | N | ASP | C | 156 | 14.823 | −4.885 | −25.208 | 1.00 | 33.80 | N |
| ATOM | 10267 | CA | ASP | C | 156 | 15.133 | −3.481 | −25.209 | 1.00 | 37.50 | C |
| ATOM | 10269 | CB | ASP | C | 156 | 16.520 | −3.167 | −25.791 | 1.00 | 43.30 | C |
| ATOM | 10272 | CG | ASP | C | 156 | 16.672 | −3.567 | −27.265 | 1.00 | 60.00 | C |
| ATOM | 10273 | OD1 | ASP | C | 156 | 17.517 | −4.457 | −27.535 | 1.00 | 63.74 | O |
| ATOM | 10274 | OD2 | ASP | C | 156 | 16.061 | −3.017 | −28.222 | 1.00 | 58.12 | O |
| ATOM | 10275 | C | ASP | C | 156 | 14.075 | −2.660 | −25.914 | 1.00 | 32.43 | C |
| ATOM | 10276 | O | ASP | C | 156 | 14.146 | −1.438 | −25.912 | 1.00 | 37.68 | O |
| ATOM | 10277 | N | ALA | C | 157 | 13.130 | −3.309 | −26.561 | 1.00 | 32.47 | N |
| ATOM | 10279 | CA | ALA | C | 157 | 12.086 | −2.590 | −27.286 | 1.00 | 38.16 | C |
| ATOM | 10281 | CB | ALA | C | 157 | 12.341 | −2.729 | −28.798 | 1.00 | 36.81 | C |
| ATOM | 10285 | C | ALA | C | 157 | 10.687 | −3.137 | −26.915 | 1.00 | 35.84 | C |
| ATOM | 10286 | O | ALA | C | 157 | 10.569 | −4.336 | −26.633 | 1.00 | 40.87 | O |
| ATOM | 10287 | N | VAL | C | 158 | 9.665 | −2.262 | −26.924 | 1.00 | 39.35 | N |
| ATOM | 10289 | CA | VAL | C | 158 | 8.251 | −2.605 | −26.632 | 1.00 | 31.93 | C |
| ATOM | 10291 | CB | VAL | C | 158 | 7.738 | −1.921 | −25.306 | 1.00 | 36.48 | C |
| ATOM | 10293 | CG1 | VAL | C | 158 | 7.610 | −0.430 | −25.446 | 1.00 | 29.19 | C |
| ATOM | 10297 | CG2 | VAL | C | 158 | 6.372 | −2.463 | −24.838 | 1.00 | 32.61 | C |
| ATOM | 10301 | C | VAL | C | 158 | 7.373 | −2.215 | −27.818 | 1.00 | 32.50 | C |
| ATOM | 10302 | O | VAL | C | 158 | 7.425 | −1.103 | −28.353 | 1.00 | 28.85 | O |
| ATOM | 10303 | N | VAL | C | 159 | 6.656 | −3.198 | −28.329 | 1.00 | 30.13 | N |
| ATOM | 10305 | CA | VAL | C | 159 | 5.605 | −2.945 | −29.276 | 1.00 | 29.22 | C |
| ATOM | 10307 | CB | VAL | C | 159 | 5.434 | −4.179 | −30.170 | 1.00 | 26.09 | C |
| ATOM | 10309 | CG1 | VAL | C | 159 | 4.452 | −3.889 | −31.246 | 1.00 | 26.83 | C |
| ATOM | 10313 | CG2 | VAL | C | 159 | 6.806 | −4.629 | −30.735 | 1.00 | 31.25 | C |
| ATOM | 10317 | C | VAL | C | 159 | 4.297 | −2.644 | −28.539 | 1.00 | 27.42 | C |
| ATOM | 10318 | O | VAL | C | 159 | 3.786 | −3.478 | −27.786 | 1.00 | 35.50 | O |
| ATOM | 10319 | N | ILE | C | 160 | 3.755 | −1.464 | −28.808 | 1.00 | 32.97 | N |
| ATOM | 10321 | CA | ILE | C | 160 | 2.443 | −1.048 | −28.308 | 1.00 | 32.91 | C |
| ATOM | 10323 | CB | ILE | C | 160 | 2.492 | 0.417 | −27.795 | 1.00 | 29.74 | C |
| ATOM | 10325 | CG1 | ILE | C | 160 | 3.703 | 0.623 | −26.868 | 1.00 | 24.45 | C |
| ATOM | 10328 | CD1 | ILE | C | 160 | 3.762 | 2.012 | −26.175 | 1.00 | 24.09 | C |
| ATOM | 10332 | CG2 | ILE | C | 160 | 1.274 | 0.692 | −26.944 | 1.00 | 33.02 | C |
| ATOM | 10336 | C | ILE | C | 160 | 1.415 | −1.236 | −29.416 | 1.00 | 37.70 | C |
| ATOM | 10337 | O | ILE | C | 160 | 1.503 | −0.621 | −30.483 | 1.00 | 36.95 | O |
| ATOM | 10338 | N | SER | C | 161 | 0.532 | −2.213 | −29.236 | 1.00 | 31.98 | N |
| ATOM | 10340 | CA | SER | C | 161 | −0.497 | −2.443 | −30.228 | 1.00 | 43.80 | C |
| ATOM | 10342 | CB | SER | C | 161 | −0.618 | −3.933 | −30.549 | 1.00 | 34.70 | C |
| ATOM | 10345 | OG | SER | C | 161 | 0.557 | −4.439 | −31.158 | 1.00 | 55.26 | O |
| ATOM | 10347 | C | SER | C | 161 | −1.803 | −1.911 | −29.630 | 1.00 | 43.95 | C |
| ATOM | 10348 | O | SER | C | 161 | −2.184 | −2.343 | −28.558 | 1.00 | 44.13 | O |
| ATOM | 10349 | N | CYS | C | 162 | −2.481 | −0.967 | −30.268 | 1.00 | 44.03 | N |
| ATOM | 10351 | CA | CYS | C | 162 | −3.874 | −0.728 | −29.862 | 1.00 | 54.75 | C |
| ATOM | 10353 | CB | CYS | C | 162 | −4.217 | 0.749 | −29.622 | 1.00 | 56.06 | C |
| ATOM | 10356 | SG | CYS | C | 162 | −3.125 | 1.714 | −28.559 | 1.00 | 82.17 | S |
| ATOM | 10357 | C | CYS | C | 162 | −4.833 | −1.244 | −30.916 | 1.00 | 51.77 | C |
| ATOM | 10358 | O | CYS | C | 162 | −4.608 | −1.081 | −32.114 | 1.00 | 44.94 | O |
| ATOM | 10359 | N | ALA | C | 163 | −5.936 | −1.825 | −30.465 | 1.00 | 55.47 | N |
| ATOM | 10361 | CA | ALA | C | 163 | −7.113 | −1.929 | −31.324 | 1.00 | 57.53 | C |
| ATOM | 10363 | CB | ALA | C | 163 | −7.110 | −3.268 | −32.041 | 1.00 | 59.26 | C |
| ATOM | 10367 | C | ALA | C | 163 | −8.434 | −1.695 | −30.563 | 1.00 | 56.80 | C |
| ATOM | 10368 | O | ALA | C | 163 | −8.443 | −1.262 | −29.402 | 1.00 | 51.99 | O |
| ATOM | 10369 | N | LYS | C | 164 | −9.549 | −1.939 | −31.244 | 1.00 | 61.59 | N |
| ATOM | 10371 | CA | LYS | C | 164 | −10.861 | −1.806 | −30.619 | 1.00 | 66.25 | C |
| ATOM | 10373 | CB | LYS | C | 164 | −11.984 | −2.058 | −31.631 | 1.00 | 66.43 | C |
| ATOM | 10376 | CG | LYS | C | 164 | −12.276 | −0.835 | −32.525 | 1.00 | 68.78 | C |
| ATOM | 10379 | CD | LYS | C | 164 | −13.478 | −0.006 | −32.051 | 1.00 | 72.11 | C |
| ATOM | 10382 | CE | LYS | C | 164 | −13.551 | 1.388 | −32.698 | 1.00 | 75.61 | C |
| ATOM | 10385 | NZ | LYS | C | 164 | −13.195 | 2.531 | −31.778 | 1.00 | 62.24 | N |
| ATOM | 10389 | C | LYS | C | 164 | −10.958 | −2.704 | −29.386 | 1.00 | 63.53 | C |
| ATOM | 10390 | O | LYS | C | 164 | −11.356 | −2.230 | −28.310 | 1.00 | 65.17 | O |
| ATOM | 10391 | N | ASP | C | 165 | −10.462 | −3.936 | −29.506 | 1.00 | 58.41 | N |
| ATOM | 10393 | CA | ASP | C | 165 | −10.436 | −4.876 | −28.381 | 1.00 | 61.82 | C |
| ATOM | 10395 | CB | ASP | C | 165 | −10.710 | −6.302 | −28.875 | 1.00 | 62.33 | C |
| ATOM | 10398 | CG | ASP | C | 165 | −11.989 | −6.924 | −28.257 | 1.00 | 84.29 | C |
| ATOM | 10399 | OD1 | ASP | C | 165 | −13.021 | −6.219 | −28.066 | 1.00 | 81.79 | O |
| ATOM | 10400 | OD2 | ASP | C | 165 | −12.063 | −8.142 | −27.950 | 1.00 | 91.74 | O |
| ATOM | 10401 | C | ASP | C | 165 | −9.174 | −4.867 | −27.498 | 1.00 | 67.57 | C |
| ATOM | 10402 | O | ASP | C | 165 | −8.810 | −5.910 | −26.940 | 1.00 | 72.09 | O |
| ATOM | 10403 | N | GLY | C | 166 | −8.521 | −3.709 | −27.352 | 1.00 | 66.55 | N |
| ATOM | 10405 | CA | GLY | C | 166 | −7.587 | −3.463 | −26.255 | 1.00 | 59.01 | C |
| ATOM | 10408 | C | GLY | C | 166 | −6.257 | −2.798 | −26.624 | 1.00 | 58.68 | C |
| ATOM | 10409 | O | GLY | C | 166 | −6.032 | −2.392 | −27.762 | 1.00 | 59.76 | O |
| ATOM | 10410 | N | VAL | C | 167 | −5.381 | −2.663 | −25.634 | 1.00 | 51.44 | N |
| ATOM | 10412 | CA | VAL | C | 167 | −3.981 | −2.338 | −25.829 | 1.00 | 46.08 | C |
| ATOM | 10414 | CB | VAL | C | 167 | −3.587 | −1.053 | −25.041 | 1.00 | 49.53 | C |
| ATOM | 10416 | CG1 | VAL | C | 167 | −3.874 | −1.210 | −23.556 | 1.00 | 58.58 | C |
| ATOM | 10420 | CG2 | VAL | C | 167 | −2.123 | −0.675 | −25.253 | 1.00 | 57.34 | C |
| ATOM | 10424 | C | VAL | C | 167 | −3.121 | −3.493 | −25.346 | 1.00 | 42.73 | C |
| ATOM | 10425 | O | VAL | C | 167 | −3.467 | −4.166 | −24.374 | 1.00 | 35.90 | O |
| ATOM | 10426 | N | LYS | C | 168 | −1.961 | −3.651 | −25.990 | 1.00 | 42.21 | N |
| ATOM | 10428 | CA | LYS | C | 168 | −1.004 | −4.708 | −25.716 | 1.00 | 32.55 | C |
| ATOM | 10430 | CB | LYS | C | 168 | −1.239 | −5.841 | −26.726 | 1.00 | 33.14 | C |
| ATOM | 10433 | CG | LYS | C | 168 | −0.476 | −7.179 | −26.482 | 1.00 | 43.87 | C |
| ATOM | 10436 | CD | LYS | C | 168 | 0.159 | −7.755 | −27.788 | 1.00 | 62.74 | C |
| ATOM | 10439 | CE | LYS | C | 168 | 1.641 | −7.318 | −28.034 | 1.00 | 61.21 | C |
| ATOM | 10442 | NZ | LYS | C | 168 | 1.887 | −6.012 | −28.796 | 1.00 | 48.59 | N |
| ATOM | 10446 | C | LYS | C | 168 | 0.441 | −4.187 | −25.799 | 1.00 | 36.18 | C |
| ATOM | 10447 | O | LYS | C | 168 | 0.824 | −3.473 | −26.731 | 1.00 | 40.08 | O |
| ATOM | 10448 | N | PHE | C | 169 | 1.233 | −4.543 | −24.798 | 1.00 | 30.39 | N |
| ATOM | 10450 | CA | PHE | C | 169 | 2.629 | −4.242 | −24.756 | 1.00 | 31.84 | C |
| ATOM | 10452 | CB | PHE | C | 169 | 2.953 | −3.579 | −23.444 | 1.00 | 28.73 | C |
| ATOM | 10455 | CG | PHE | C | 169 | 2.124 | −2.376 | −23.166 | 1.00 | 33.34 | C |
| ATOM | 10456 | CD1 | PHE | C | 169 | 0.878 | −2.504 | −22.584 | 1.00 | 24.52 | C |
| ATOM | 10458 | CE1 | PHE | C | 169 | 0.113 | −1.417 | −22.351 | 1.00 | 20.94 | C |
| ATOM | 10460 | CZ | PHE | C | 169 | 0.588 | −0.163 | −22.581 | 1.00 | 25.57 | C |
| ATOM | 10462 | CE2 | PHE | C | 169 | 1.849 | −0.015 | −23.160 | 1.00 | 28.09 | C |
| ATOM | 10464 | CD2 | PHE | C | 169 | 2.622 | −1.118 | −23.391 | 1.00 | 30.42 | C |
| ATOM | 10466 | C | PHE | C | 169 | 3.446 | −5.521 | −24.846 | 1.00 | 35.66 | C |
| ATOM | 10467 | O | PHE | C | 169 | 3.347 | −6.341 | −23.951 | 1.00 | 34.42 | O |
| ATOM | 10468 | N | SER | C | 170 | 4.326 | −5.611 | −25.850 | 1.00 | 30.25 | N |
| ATOM | 10470 | CA | SER | C | 170 | 5.098 | −6.832 | −26.142 | 1.00 | 33.20 | C |
| ATOM | 10472 | CB | SER | C | 170 | 4.658 | −7.402 | −27.501 | 1.00 | 25.85 | C |
| ATOM | 10475 | OG | SER | C | 170 | 3.904 | −8.596 | −27.353 | 1.00 | 59.31 | O |
| ATOM | 10477 | C | SER | C | 170 | 6.595 | −6.488 | −26.227 | 1.00 | 33.24 | C |
| ATOM | 10478 | O | SER | C | 170 | 6.987 | −5.517 | −26.861 | 1.00 | 34.32 | O |
| ATOM | 10479 | N | ALA | C | 171 | 7.449 | −7.296 | −25.626 | 1.00 | 34.82 | N |
| ATOM | 10481 | CA | ALA | C | 171 | 8.876 | −7.129 | −25.834 | 1.00 | 40.45 | C |
| ATOM | 10483 | CB | ALA | C | 171 | 9.507 | −6.465 | −24.645 | 1.00 | 36.26 | C |
| ATOM | 10487 | C | ALA | C | 171 | 9.532 | −8.473 | −26.124 | 1.00 | 41.04 | C |
| ATOM | 10488 | O | ALA | C | 171 | 8.985 | −9.537 | −25.821 | 1.00 | 35.14 | O |
| ATOM | 10489 | N | SER | C | 172 | 10.748 | −8.394 | −26.659 | 1.00 | 46.58 | N |
| ATOM | 10491 | CA | SER | C | 172 | 11.405 | −9.530 | −27.272 | 1.00 | 43.06 | C |
| ATOM | 10493 | CB | SER | C | 172 | 11.017 | −9.537 | −28.739 | 1.00 | 47.81 | C |
| ATOM | 10496 | OG | SER | C | 172 | 10.938 | −10.864 | −29.133 | 1.00 | 45.44 | O |
| ATOM | 10498 | C | SER | C | 172 | 12.925 | −9.466 | −27.218 | 1.00 | 46.52 | C |
| ATOM | 10499 | O | SER | C | 172 | 13.522 | −8.464 | −27.600 | 1.00 | 43.64 | O |
| ATOM | 10500 | N | GLY | C | 173 | 13.556 | −10.575 | −26.842 | 1.00 | 43.56 | N |
| ATOM | 10502 | CA | GLY | C | 173 | 14.982 | −10.561 | −26.632 | 1.00 | 45.12 | C |
| ATOM | 10505 | C | GLY | C | 173 | 15.589 | −11.936 | −26.655 | 1.00 | 44.46 | C |
| ATOM | 10506 | O | GLY | C | 173 | 14.951 | −12.883 | −27.087 | 1.00 | 39.49 | O |
| ATOM | 10507 | N | GLU | C | 174 | 16.814 | −12.021 | −26.141 | 1.00 | 47.03 | N |
| ATOM | 10509 | CA | GLU | C | 174 | 17.621 | −13.237 | −26.161 | 1.00 | 48.84 | C |
| ATOM | 10511 | CB | GLU | C | 174 | 19.045 | −12.957 | −25.659 | 1.00 | 54.35 | C |
| ATOM | 10514 | CG | GLU | C | 174 | 19.924 | −12.255 | −26.681 | 1.00 | 67.07 | C |
| ATOM | 10517 | CD | GLU | C | 174 | 19.536 | −10.794 | −26.859 | 1.00 | 86.42 | C |
| ATOM | 10518 | OE1 | GLU | C | 174 | 19.665 | −10.018 | −25.880 | 1.00 | 90.44 | O |
| ATOM | 10519 | OE2 | GLU | C | 174 | 19.051 | −10.434 | −27.960 | 1.00 | 78.80 | O |
| ATOM | 10520 | C | GLU | C | 174 | 17.026 | −14.369 | −25.330 | 1.00 | 39.85 | C |
| ATOM | 10521 | O | GLU | C | 174 | 17.080 | −15.518 | −25.728 | 1.00 | 38.78 | O |
| ATOM | 10522 | N | LEU | C | 175 | 16.463 | −14.039 | −24.180 | 1.00 | 43.01 | N |
| ATOM | 10524 | CA | LEU | C | 175 | 15.851 | −15.029 | −23.301 | 1.00 | 47.39 | C |
| ATOM | 10526 | CB | LEU | C | 175 | 16.047 | −14.625 | −21.838 | 1.00 | 46.84 | C |
| ATOM | 10529 | CG | LEU | C | 175 | 15.406 | −13.309 | −21.427 | 1.00 | 54.06 | C |
| ATOM | 10531 | CD1 | LEU | C | 175 | 14.014 | −13.535 | −20.837 | 1.00 | 48.10 | C |
| ATOM | 10535 | CD2 | LEU | C | 175 | 16.331 | −12.593 | −20.447 | 1.00 | 73.59 | C |
| ATOM | 10539 | C | LEU | C | 175 | 14.364 | −15.259 | −23.547 | 1.00 | 40.65 | C |
| ATOM | 10540 | O | LEU | C | 175 | 13.794 | −16.118 | −22.929 | 1.00 | 38.76 | O |
| ATOM | 10541 | N | GLY | C | 176 | 13.728 | −14.490 | −24.426 | 1.00 | 39.18 | N |
| ATOM | 10543 | CA | GLY | C | 176 | 12.458 | −14.894 | −24.969 | 1.00 | 32.09 | C |
| ATOM | 10546 | C | GLY | C | 176 | 11.623 | −13.663 | −25.212 | 1.00 | 36.93 | C |
| ATOM | 10547 | O | GLY | C | 176 | 12.178 | −12.650 | −25.619 | 1.00 | 29.14 | O |
| ATOM | 10548 | N | ASN | C | 177 | 10.332 | −13.741 | −24.893 | 1.00 | 31.12 | N |
| ATOM | 10550 | CA | ASN | C | 177 | 9.443 | −12.616 | −25.042 | 1.00 | 34.53 | C |
| ATOM | 10552 | CB | ASN | C | 177 | 8.976 | −12.679 | −26.484 | 1.00 | 29.12 | C |
| ATOM | 10555 | CG | ASN | C | 177 | 8.359 | −13.998 | −26.829 | 1.00 | 41.73 | C |
| ATOM | 10556 | OD1 | ASN | C | 177 | 7.158 | −14.179 | −26.667 | 1.00 | 62.01 | O |
| ATOM | 10557 | ND2 | ASN | C | 177 | 9.185 | −14.958 | −27.217 | 1.00 | 77.07 | N |
| ATOM | 10560 | C | ASN | C | 177 | 8.273 | −12.537 | −24.005 | 1.00 | 33.51 | C |
| ATOM | 10661 | O | ASN | C | 177 | 7.991 | −13.449 | −23.242 | 1.00 | 36.83 | O |
| ATOM | 10562 | N | GLY | C | 178 | 7.597 | −11.414 | −23.934 | 1.00 | 39.97 | N |
| ATOM | 10564 | CA | GLY | C | 178 | 6.461 | −11.315 | −23.041 | 1.00 | 36.01 | C |
| ATOM | 10567 | C | GLY | C | 178 | 5.438 | −10.418 | −23.677 | 1.00 | 36.07 | C |
| ATOM | 10568 | O | GLY | C | 178 | 5.814 | −9.615 | −24.522 | 1.00 | 33.87 | O |
| ATOM | 10569 | N | ASN | C | 179 | 4.162 | −10.605 | −23.313 | 1.00 | 40.88 | N |
| ATOM | 10571 | CA | ASN | C | 179 | 3.038 | −9.768 | −23.761 | 1.00 | 37.04 | C |
| ATOM | 10573 | CB | ASN | C | 179 | 2.146 | −10.479 | −24.784 | 1.00 | 38.36 | C |
| ATOM | 10576 | CG | ASN | C | 179 | 2.844 | −11.621 | −25.492 | 1.00 | 61.06 | C |
| ATOM | 10577 | OD1 | ASN | C | 179 | 2.488 | −12.778 | −25.273 | 1.00 | 66.75 | O |
| ATOM | 10578 | ND2 | ASN | C | 179 | 3.837 | −11.313 | −26.345 | 1.00 | 67.48 | N |
| ATOM | 10581 | C | ASN | C | 179 | 2.160 | −9.418 | −22.551 | 1.00 | 38.84 | C |
| ATOM | 10582 | O | ASN | C | 179 | 1.700 | −10.289 | −21.835 | 1.00 | 36.64 | O |
| ATOM | 10583 | N | ILE | C | 180 | 1.881 | −8.141 | −22.352 | 1.00 | 39.92 | N |
| ATOM | 10585 | CA | ILE | C | 180 | 0.996 | −7.697 | −21.292 | 1.00 | 37.01 | C |
| ATOM | 10587 | CB | ILE | C | 180 | 1.753 | −6.697 | −20.426 | 1.00 | 33.29 | C |
| ATOM | 10589 | CG1 | ILE | C | 180 | 3.023 | −7.352 | −19.896 | 1.00 | 38.68 | C |
| ATOM | 10592 | CD1 | ILE | C | 180 | 3.681 | −6.582 | −18.791 | 1.00 | 30.36 | C |
| ATOM | 10596 | CG2 | ILE | C | 180 | 0.861 | −6.177 | −19.306 | 1.00 | 40.65 | C |
| ATOM | 10600 | C | ILE | C | 180 | −0.222 | −7.013 | −21.895 | 1.00 | 39.19 | C |
| ATOM | 10601 | O | ILE | C | 180 | −0.098 | −5.997 | −22.581 | 1.00 | 39.80 | O |
| ATOM | 10602 | N | LYS | C | 181 | −1.386 | −7.610 | −21.667 | 1.00 | 39.58 | N |
| ATOM | 10604 | CA | LYS | C | 181 | −2.661 | −7.141 | −22.211 | 1.00 | 35.13 | C |
| ATOM | 10606 | CB | LYS | C | 181 | −3.439 | −8.322 | −22.821 | 1.00 | 40.46 | C |
| ATOM | 10609 | CG | LYS | C | 181 | −4.906 | −8.027 | −23.154 | 1.00 | 55.20 | C |
| ATOM | 10612 | CD | LYS | C | 181 | −5.111 | −6.770 | −24.023 | 1.00 | 58.47 | C |
| ATOM | 10615 | CE | LYS | C | 181 | −6.362 | −6.883 | −24.903 | 1.00 | 59.32 | C |
| ATOM | 10618 | NZ | LYS | C | 181 | −6.073 | −6.880 | −26.383 | 1.00 | 60.63 | N |
| ATOM | 10622 | C | LYS | C | 181 | −3.457 | −6.519 | −21.055 | 1.00 | 35.27 | C |
| ATOM | 10623 | O | LYS | C | 181 | −3.726 | −7.186 | −20.037 | 1.00 | 40.57 | O |
| ATOM | 10624 | N | LEU | C | 182 | −3.785 | −5.233 | −21.197 | 1.00 | 32.96 | N |
| ATOM | 10626 | CA | LEU | C | 182 | −4.476 | −4.479 | −20.168 | 1.00 | 39.28 | C |
| ATOM | 10628 | CB | LEU | C | 182 | −3.699 | −3.220 | −19.841 | 1.00 | 40.98 | C |
| ATOM | 10631 | CG | LEU | C | 182 | −3.621 | −2.603 | −18.446 | 1.00 | 54.41 | C |
| ATOM | 10633 | CD1 | LEU | C | 182 | −3.653 | −1.090 | −18.627 | 1.00 | 48.00 | C |
| ATOM | 10637 | CD2 | LEU | C | 182 | −4.729 | −2.982 | −17.477 | 1.00 | 64.35 | C |
| ATOM | 10641 | C | LEU | C | 182 | −5.862 | −4.108 | −20.682 | 1.00 | 39.48 | C |
| ATOM | 10642 | O | LEU | C | 182 | −6.007 | −3.480 | −21.719 | 1.00 | 44.69 | O |
| ATOM | 10643 | N | SER | C | 183 | −6.903 | −4.586 | −20.018 | 1.00 | 41.07 | N |
| ATOM | 10645 | CA | SER | C | 183 | −8.237 | −4.452 | −20.624 | 1.00 | 50.30 | C |
| ATOM | 10647 | CB | SER | C | 183 | −9.217 | −5.544 | −20.162 | 1.00 | 54.37 | C |
| ATOM | 10650 | OG | SER | C | 183 | −9.295 | −5.577 | −18.758 | 1.00 | 58.89 | O |
| ATOM | 10652 | C | SER | C | 183 | −8.786 | −3.076 | −20.315 | 1.00 | 38.55 | C |
| ATOM | 10653 | O | SER | C | 183 | −8.518 | −2.549 | −19.250 | 1.00 | 32.47 | O |
| ATOM | 10654 | N | GLN | C | 184 | −9.464 | −2.451 | −21.268 | 1.00 | 41.11 | N |
| ATOM | 10656 | CA | GLN | C | 184 | −10.350 | −1.343 | −20.936 | 1.00 | 45.33 | C |
| ATOM | 10658 | CB | GLN | C | 184 | −11.170 | −0.910 | −22.150 | 1.00 | 51.37 | C |
| ATOM | 10661 | CG | GLN | C | 184 | −10.317 | −0.702 | −23.409 | 1.00 | 49.76 | C |
| ATOM | 10664 | CD | GLN | C | 184 | −11.096 | −0.797 | −24.732 | 1.00 | 41.40 | C |
| ATOM | 10665 | OE1 | GLN | C | 184 | −11.209 | −1.873 | −25.327 | 1.00 | 61.59 | O |
| ATOM | 10666 | NE2 | GLN | C | 184 | −11.540 | 0.352 | −25.231 | 1.00 | 58.46 | N |
| ATOM | 10669 | C | GLN | C | 184 | −11.278 | −1.669 | −19.758 | 1.00 | 46.08 | C |
| ATOM | 10670 | O | GLN | C | 184 | −11.634 | −2.815 | −19.477 | 1.00 | 49.20 | O |
| ATOM | 10671 | N | THR | C | 185 | −11.567 | −0.643 | −18.982 | 1.00 | 49.29 | N |
| ATOM | 10673 | CA | THR | C | 185 | −12.575 | −0.745 | −17.943 | 1.00 | 52.89 | C |
| ATOM | 10675 | CB | THR | C | 185 | −12.120 | 0.080 | −16.766 | 1.00 | 51.20 | C |
| ATOM | 10677 | OG1 | THR | C | 185 | −11.904 | 1.425 | −17.220 | 1.00 | 45.71 | O |
| ATOM | 10679 | CG2 | THR | C | 185 | −10.793 | −0.422 | −16.204 | 1.00 | 60.92 | C |
| ATOM | 10683 | C | THR | C | 185 | −13.880 | −0.143 | −18.458 | 1.00 | 58.43 | C |
| ATOM | 10684 | O | THR | C | 185 | −13.899 | 0.511 | −19.504 | 1.00 | 65.98 | O |
| ATOM | 10685 | N | SER | C | 186 | −14.964 | −0.325 | −17.712 | 1.00 | 61.48 | N |
| ATOM | 10687 | CA | SER | C | 186 | −16.218 | 0.326 | −18.072 | 1.00 | 67.01 | C |
| ATOM | 10689 | CB | SER | C | 186 | −17.320 | −0.690 | −18.353 | 1.00 | 65.86 | C |
| ATOM | 10692 | OG | SER | C | 186 | −18.331 | −0.060 | −19.113 | 1.00 | 68.09 | O |
| ATOM | 10694 | C | SER | C | 186 | −16.669 | 1.276 | −16.978 | 1.00 | 75.47 | C |
| ATOM | 10695 | O | SER | C | 186 | −16.558 | 2.500 | −17.114 | 1.00 | 79.28 | O |
| ATOM | 10696 | N | ASN | C | 187 | −17.202 | 0.709 | −15.901 | 1.00 | 82.02 | N |
| ATOM | 10698 | CA | ASN | C | 187 | −17.506 | 1.494 | −14.713 | 1.00 | 89.39 | C |
| ATOM | 10700 | CB | ASN | C | 187 | −18.941 | 1.243 | −14.242 | 1.00 | 88.62 | C |
| ATOM | 10703 | CG | ASN | C | 187 | −19.485 | 2.394 | −13.409 | 1.00 | 95.82 | C |
| ATOM | 10704 | OD1 | ASN | C | 187 | −19.594 | 3.529 | −13.884 | 1.00 | 95.12 | O |
| ATOM | 10705 | ND2 | ASN | C | 187 | −19.811 | 2.110 | −12.150 | 1.00 | 99.11 | N |
| ATOM | 10708 | C | ASN | C | 187 | −16.515 | 1.154 | −13.609 | 1.00 | 91.91 | C |
| ATOM | 10709 | O | ASN | C | 187 | −16.688 | 0.156 | −12.910 | 1.00 | 96.07 | O |
| ATOM | 10710 | N | VAL | C | 188 | −15.458 | 1.954 | −13.490 | 1.00 | 98.09 | N |
| ATOM | 10712 | CA | VAL | C | 188 | −14.381 | 1.665 | −12.545 | 1.00 | 101.29 | C |
| ATOM | 10714 | CB | VAL | C | 188 | −12.992 | 2.085 | −13.068 | 1.00 | 98.28 | C |
| ATOM | 10716 | CG1 | VAL | C | 188 | −11.903 | 1.316 | −12.336 | 1.00 | 94.43 | C |
| ATOM | 10720 | CG2 | VAL | C | 188 | −12.892 | 1.848 | −14.554 | 1.00 | 98.51 | C |
| ATOM | 10724 | C | VAL | C | 188 | −14.619 | 2.369 | −11.218 | 1.00 | 106.23 | C |
| ATOM | 10725 | C | VAL | C | 188 | −14.192 | 1.874 | −10.172 | 1.00 | 111.19 | O |
| ATOM | 10726 | N | ASP | C | 189 | −15.278 | 3.525 | −11.265 | 1.00 | 109.04 | N |
| ATOM | 10728 | CA | ASP | C | 189 | −15.678 | 4.242 | −10.052 | 1.00 | 112.21 | C |
| ATOM | 10730 | CB | ASP | C | 189 | −16.569 | 3.355 | −9.163 | 1.00 | 113.89 | C |
| ATOM | 10733 | CG | ASP | C | 189 | −16.187 | 3.412 | −7.688 | 1.00 | 120.90 | C |
| ATOM | 10734 | OD1 | ASP | C | 189 | −17.011 | 3.933 | −6.883 | 1.00 | 129.83 | O |
| ATOM | 10735 | OD2 | ASP | C | 189 | −15.107 | 2.942 | −7.239 | 1.00 | 120.13 | O |
| ATOM | 10736 | C | ASP | C | 189 | −14.469 | 4.783 | −9.278 | 1.00 | 112.29 | C |
| ATOM | 10737 | O | ASP | C | 189 | −13.974 | 4.155 | −8.334 | 1.00 | 110.04 | O |
| ATOM | 10738 | N | LYS | C | 190 | −14.008 | 5.957 | −9.711 | 1.00 | 112.53 | N |
| ATOM | 10740 | CA | LYS | C | 190 | −12.916 | 6.697 | −9.076 | 1.00 | 110.49 | C |
| ATOM | 10742 | CB | LYS | C | 190 | −11.805 | 5.769 | −8.567 | 1.00 | 111.84 | C |
| ATOM | 10745 | CG | LYS | C | 190 | −11.498 | 5.928 | −7.064 | 1.00 | 115.96 | C |
| ATOM | 10748 | CD | LYS | C | 190 | −11.569 | 4.556 | −6.321 | 1.00 | 120.09 | C |
| ATOM | 10751 | CE | LYS | C | 190 | −10.367 | 4.390 | −5.384 | 1.00 | 121.28 | C |
| ATOM | 10754 | NZ | LYS | C | 190 | −9.407 | 3.347 | −5.876 | 1.00 | 123.09 | N |
| ATOM | 10758 | C | LYS | C | 190 | −12.350 | 7.685 | −10.093 | 1.00 | 105.84 | C |
| ATOM | 10759 | O | LYS | C | 190 | −11.947 | 8.791 | −9.726 | 1.00 | 110.63 | O |
| ATOM | 10760 | N | GLU | C | 191 | −12.352 | 7.285 | −11.366 | 1.00 | 96.91 | N |
| ATOM | 10762 | CA | GLU | C | 191 | −11.890 | 8.122 | −12.473 | 1.00 | 92.22 | C |
| ATOM | 10764 | CB | GLU | C | 191 | −12.037 | 9.613 | −12.126 | 1.00 | 96.00 | C |
| ATOM | 10767 | CG | GLU | C | 191 | −11.658 | 10.601 | −13.226 | 1.00 | 102.22 | C |
| ATOM | 10770 | CD | GLU | C | 191 | −10.838 | 11.777 | −12.700 | 1.00 | 114.79 | C |
| ATOM | 10771 | OE1 | GLU | C | 191 | −11.426 | 12.713 | −12.105 | 1.00 | 112.07 | O |
| ATOM | 10772 | OE2 | GLU | C | 191 | −9.597 | 11.764 | −12.877 | 1.00 | 118.67 | O |
| ATOM | 10773 | C | GLU | C | 191 | −10.438 | 7.774 | −12.826 | 1.00 | 84.93 | C |
| ATOM | 10774 | O | GLU | C | 191 | −10.104 | 7.635 | −14.001 | 1.00 | 78.90 | O |
| ATOM | 10775 | N | GLU | C | 192 | −9.604 | 7.590 | −11.800 | 1.00 | 78.09 | N |
| ATOM | 10777 | CA | GLU | C | 192 | −8.157 | 7.444 | −11.944 | 1.00 | 71.32 | C |
| ATOM | 10779 | CB | GLU | C | 192 | −7.443 | 8.046 | −10.726 | 1.00 | 71.18 | C |
| ATOM | 10782 | CG | GLU | C | 192 | −7.568 | 9.560 | −10.610 | 1.00 | 84.65 | C |
| ATOM | 10785 | CD | GLU | C | 192 | −8.455 | 9.992 | −9.451 | 1.00 | 99.12 | C |
| ATOM | 10786 | OE1 | GLU | C | 192 | −8.692 | 11.210 | −9.315 | 1.00 | 107.43 | O |
| ATOM | 10787 | OE2 | GLU | C | 192 | −8.916 | 9.125 | −8.672 | 1.00 | 108.20 | O |
| ATOM | 10788 | C | GLU | C | 192 | −7.734 | 5.982 | −12.103 | 1.00 | 62.75 | C |
| ATOM | 10789 | O | GLU | C | 192 | −6.547 | 5.674 | −12.249 | 1.00 | 52.18 | O |
| ATOM | 10790 | N | GLU | C | 193 | −8.704 | 5.080 | −12.049 | 1.00 | 52.45 | N |
| ATOM | 10792 | CA | GLU | C | 193 | −8.420 | 3.682 | −12.287 | 1.00 | 57.86 | C |
| ATOM | 10794 | CB | GLU | C | 193 | −9.140 | 2.851 | −11.239 | 1.00 | 61.28 | C |
| ATOM | 10797 | CG | GLU | C | 193 | −8.393 | 2.751 | −9.917 | 1.00 | 73.56 | C |
| ATOM | 10800 | CD | GLU | C | 193 | −8.753 | 1.487 | −9.143 | 1.00 | 98.60 | C |
| ATOM | 10801 | OE1 | GLU | C | 193 | −9.968 | 1.205 | −8.997 | 1.00 | 104.37 | O |
| ATOM | 10802 | OE2 | GLU | C | 193 | −7.832 | 0.762 | −8.690 | 1.00 | 98.73 | O |
| ATOM | 10803 | C | GLU | C | 193 | −8.777 | 3.233 | −13.714 | 1.00 | 56.68 | C |
| ATOM | 10804 | O | GLU | C | 193 | −8.565 | 2.062 | −14.089 | 1.00 | 51.65 | O |
| ATOM | 10805 | N | ALA | C | 194 | −9.259 | 4.182 | −14.517 | 1.00 | 44.98 | N |
| ATOM | 10807 | CA | ALA | C | 194 | −9.833 | 3.860 | −15.807 | 1.00 | 49.17 | C |
| ATOM | 10809 | CB | ALA | C | 194 | −10.677 | 5.007 | −16.293 | 1.00 | 55.22 | C |
| ATOM | 10813 | C | ALA | C | 194 | −8.732 | 3.560 | −16.812 | 1.00 | 49.76 | C |
| ATOM | 10814 | O | ALA | C | 194 | −7.635 | 4.127 | −16.752 | 1.00 | 49.83 | O |
| ATOM | 10815 | N | VAL | C | 195 | −9.030 | 2.624 | −17.704 | 1.00 | 48.07 | N |
| ATOM | 10817 | CA | VAL | C | 195 | −8.231 | 2.394 | −18.887 | 1.00 | 36.80 | C |
| ATOM | 10819 | CB | VAL | C | 195 | −7.652 | 0.960 | −18.932 | 1.00 | 46.91 | C |
| ATOM | 10821 | CG1 | VAL | C | 195 | −6.973 | 0.728 | −20.254 | 1.00 | 35.19 | C |
| ATOM | 10825 | CG2 | VAL | C | 195 | −6.687 | 0.680 | −17.778 | 1.00 | 41.92 | C |
| ATOM | 10829 | C | VAL | C | 195 | −9.178 | 2.605 | −20.069 | 1.00 | 37.20 | C |
| ATOM | 10830 | O | VAL | C | 195 | −10.144 | 1.850 | −20.275 | 1.00 | 37.54 | O |
| ATOM | 10831 | N | THR | C | 196 | −8.940 | 3.694 | −20.792 | 1.00 | 32.94 | N |
| ATOM | 10833 | CA | THR | C | 196 | −9.768 | 4.078 | −21.930 | 1.00 | 36.38 | C |
| ATOM | 10835 | CB | THR | C | 196 | −10.611 | 5.299 | −21.604 | 1.00 | 39.76 | C |
| ATOM | 10837 | OG1 | THR | C | 196 | −11.097 | 5.179 | −20.258 | 1.00 | 37.29 | O |
| ATOM | 10839 | CG2 | THR | C | 196 | −11.902 | 5.300 | −22.441 | 1.00 | 52.29 | C |
| ATOM | 10843 | C | THR | C | 196 | −8.962 | 4.270 | −23.200 | 1.00 | 38.22 | C |
| ATOM | 10844 | O | THR | C | 196 | −7.827 | 4.739 | −23.178 | 1.00 | 39.15 | O |
| ATOM | 10845 | N | ILE | C | 197 | −9.495 | 3.717 | −24.276 | 1.00 | 41.54 | N |
| ATOM | 10847 | CA | ILE | C | 197 | −8.875 | 3.841 | −25.575 | 1.00 | 37.54 | C |
| ATOM | 10849 | CB | ILE | C | 197 | −8.545 | 2.475 | −26.140 | 1.00 | 37.67 | C |
| ATOM | 10851 | CG1 | ILE | C | 197 | −7.607 | 1.718 | −25.197 | 1.00 | 39.61 | C |
| ATOM | 10854 | CD1 | ILE | C | 197 | −7.604 | 0.213 | −25.449 | 1.00 | 53.52 | C |
| ATOM | 10858 | CG2 | ILE | C | 197 | −7.917 | 2.648 | −27.510 | 1.00 | 34.80 | C |
| ATOM | 10862 | C | ILE | C | 197 | −9.735 | 4.608 | −26.548 | 1.00 | 35.28 | C |
| ATOM | 10863 | O | ILE | C | 197 | −10.814 | 4.159 | −26.941 | 1.00 | 42.08 | O |
| ATOM | 10864 | N | GLU | C | 198 | −9.242 | 5.780 | −26.924 | 1.00 | 40.32 | N |
| ATOM | 10866 | CA | GLU | C | 198 | −9.760 | 6.524 | −28.069 | 1.00 | 50.89 | C |
| ATOM | 10868 | CB | GLU | C | 198 | −9.680 | 8.022 | −27.757 | 1.00 | 44.41 | C |
| ATOM | 10871 | CG | GLU | C | 198 | −10.718 | 8.906 | −28.426 | 1.00 | 70.17 | C |
| ATOM | 10874 | CD | GLU | C | 198 | −11.068 | 10.114 | −27.566 | 1.00 | 95.21 | C |
| ATOM | 10875 | OE1 | GLU | C | 198 | −10.172 | 10.964 | −27.335 | 1.00 | 94.41 | O |
| ATOM | 10876 | OE2 | GLU | C | 198 | −12.233 | 10.199 | −27.100 | 1.00 | 103.12 | O |
| ATOM | 10877 | C | GLU | C | 198 | −9.029 | 6.149 | −29.385 | 1.00 | 54.57 | C |
| ATOM | 10878 | O | GLU | C | 198 | −7.922 | 6.618 | −29.700 | 1.00 | 50.90 | O |
| ATOM | 10879 | N | MET | C | 199 | −9.700 | 5.326 | −30.185 | 1.00 | 58.15 | N |
| ATOM | 10881 | CA | MET | C | 199 | −9.094 | 4.616 | −31.310 | 1.00 | 57.76 | C |
| ATOM | 10883 | CB | MET | C | 199 | −9.366 | 3.122 | −31.091 | 1.00 | 65.16 | C |
| ATOM | 10886 | CG | MET | C | 199 | −8.212 | 2.163 | −31.278 | 1.00 | 64.14 | C |
| ATOM | 10889 | SD | MET | C | 199 | −7.869 | 1.808 | −33.002 | 1.00 | 77.71 | S |
| ATOM | 10890 | CE | MET | C | 199 | −9.521 | 1.471 | −33.726 | 1.00 | 63.94 | C |
| ATOM | 10894 | C | MET | C | 199 | −9.721 | 5.062 | −32.656 | 1.00 | 63.63 | C |
| ATOM | 10895 | O | MET | C | 199 | −10.953 | 5.048 | −32.819 | 1.00 | 61.08 | O |
| ATOM | 10896 | N | ASN | C | 200 | −8.880 | 5.445 | −33.618 | 1.00 | 58.33 | N |
| ATOM | 10898 | CA | ASN | C | 200 | −9.327 | 5.739 | −34.986 | 1.00 | 56.60 | C |
| ATOM | 10900 | CB | ASN | C | 200 | −8.646 | 7.003 | −35.485 | 1.00 | 59.62 | C |
| ATOM | 10903 | CG | ASN | C | 200 | −9.329 | 8.269 | −34.998 | 1.00 | 56.00 | C |
| ATOM | 10904 | OD1 | ASN | C | 200 | −9.544 | 8.462 | −33.796 | 1.00 | 68.49 | O |
| ATOM | 10905 | ND2 | ASN | C | 200 | −9.712 | 9.121 | −35.940 | 1.00 | 69.71 | N |
| ATOM | 10908 | C | ASN | C | 200 | −9.031 | 4.614 | −35.983 | 1.00 | 59.73 | C |
| ATOM | 10909 | O | ASN | C | 200 | −9.893 | 4.179 | −36.751 | 1.00 | 66.30 | O |
| ATOM | 10910 | N | GLU | C | 201 | −7.792 | 4.143 | −35.975 | 1.00 | 56.52 | N |
| ATOM | 10912 | CA | GLU | C | 201 | −7.416 | 2.959 | −36.732 | 1.00 | 55.76 | C |
| ATOM | 10914 | CB | GLU | C | 201 | −6.660 | 3.380 | −38.004 | 1.00 | 59.89 | C |
| ATOM | 10917 | CG | GLU | C | 201 | −6.070 | 4.785 | −37.892 | 1.00 | 57.58 | C |
| ATOM | 10920 | CD | GLU | C | 201 | −5.174 | 5.170 | −39.061 | 1.00 | 63.81 | C |
| ATOM | 10921 | OE1 | GLU | C | 201 | −5.190 | 6.358 | −39.440 | 1.00 | 64.11 | O |
| ATOM | 10922 | OE2 | GLU | C | 201 | −4.439 | 4.308 | −39.592 | 1.00 | 44.43 | O |
| ATOM | 10923 | C | GLU | C | 201 | −6.442 | 2.251 | −35.814 | 1.00 | 53.03 | C |
| ATOM | 10924 | O | GLU | C | 201 | −5.724 | 2.924 | −35.067 | 1.00 | 53.97 | O |
| ATOM | 10925 | N | PRO | C | 202 | −6.365 | 0.928 | −35.897 | 1.00 | 45.21 | N |
| ATOM | 10926 | CA | PRO | C | 202 | −5.307 | 0.187 | −35.200 | 1.00 | 42.43 | C |
| ATOM | 10928 | CB | PRO | C | 202 | −5.473 | −1.253 | −35.696 | 1.00 | 40.83 | C |
| ATOM | 10931 | CG | PRO | C | 202 | −6.862 | −1.332 | −36.264 | 1.00 | 50.35 | C |
| ATOM | 10934 | CD | PRO | C | 202 | −7.259 | 0.054 | −38.675 | 1.00 | 46.52 | C |
| ATOM | 10937 | C | PRO | C | 202 | −3.908 | 0.704 | −35.555 | 1.00 | 46.74 | C |
| ATOM | 10938 | O | PRO | C | 202 | −3.687 | 1.271 | −36.621 | 1.00 | 48.46 | O |
| ATOM | 10939 | N | VAL | C | 203 | −3.002 | 0.568 | −34.594 | 1.00 | 44.88 | N |
| ATOM | 10941 | CA | VAL | C | 203 | −1.639 | 1.029 | −34.689 | 1.00 | 43.25 | C |
| ATOM | 10943 | CB | VAL | C | 203 | −1.458 | 2.393 | −34.018 | 1.00 | 47.39 | C |
| ATOM | 10945 | CG1 | VAL | C | 203 | −0.019 | 2.798 | −34.042 | 1.00 | 53.37 | C |
| ATOM | 10949 | CG2 | VAL | C | 203 | −2.290 | 3.431 | −34.695 | 1.00 | 45.66 | C |
| ATOM | 10953 | C | VAL | C | 203 | −0.786 | 0.010 | −33.955 | 1.00 | 42.28 | C |
| ATOM | 10954 | O | VAL | C | 203 | −1.248 | −0.654 | −33.021 | 1.00 | 46.41 | O |
| ATOM | 10955 | N | GLN | C | 204 | 0.439 | −0.134 | −34.438 | 1.00 | 44.55 | N |
| ATOM | 10957 | CA | GLN | C | 204 | 1.453 | −1.019 | −33.890 | 1.00 | 40.25 | C |
| ATOM | 10959 | CB | GLN | C | 204 | 1.473 | −2.329 | −34.687 | 1.00 | 51.92 | C |
| ATOM | 10962 | CG | GLN | C | 204 | 2.375 | −3.439 | −34.119 | 1.00 | 52.83 | C |
| ATOM | 10965 | CD | GLN | C | 204 | 2.746 | −4.481 | −35.165 | 1.00 | 63.64 | C |
| ATOM | 10966 | OE1 | GLN | C | 204 | 1.866 | −5.088 | −35.780 | 1.00 | 83.21 | O |
| ATOM | 10967 | NE2 | GLN | C | 204 | 4.047 | −4.661 | −35.397 | 1.00 | 68.55 | N |
| ATOM | 10970 | C | GLN | C | 204 | 2.771 | −0.271 | −34.081 | 1.00 | 43.28 | C |
| ATOM | 10971 | O | GLN | C | 204 | 3.164 | −0.004 | −35.226 | 1.00 | 44.00 | O |
| ATOM | 10972 | N | LEU | C | 205 | 3.417 | 0.106 | −32.973 | 1.00 | 40.51 | N |
| ATOM | 10974 | CA | LEU | C | 205 | 4.661 | 0.845 | −33.008 | 1.00 | 37.79 | C |
| ATOM | 10976 | CB | LEU | C | 205 | 4.352 | 2.328 | −32.772 | 1.00 | 46.39 | C |
| ATOM | 10979 | CG | LEU | C | 205 | 3.359 | 2.902 | −33.802 | 1.00 | 46.98 | C |
| ATOM | 10981 | CD1 | LEU | C | 205 | 2.779 | 4.213 | −33.342 | 1.00 | 36.98 | C |
| ATOM | 10985 | CD2 | LEU | C | 205 | 3.970 | 3.079 | −35.168 | 1.00 | 44.61 | C |
| ATOM | 10989 | C | LEU | C | 205 | 5.672 | 0.323 | −32.001 | 1.00 | 32.66 | C |
| ATOM | 10990 | O | LEU | C | 205 | 5.306 | −0.053 | −30.913 | 1.00 | 30.50 | O |
| ATOM | 10991 | N | THR | C | 206 | 6.951 | 0.418 | −32.347 | 1.00 | 32.71 | N |
| ATOM | 10993 | CA | THR | C | 206 | 8.079 | −0.023 | −31.547 | 1.00 | 31.46 | C |
| ATOM | 10995 | CB | THR | C | 206 | 8.971 | −0.937 | −32.428 | 1.00 | 33.34 | C |
| ATOM | 10997 | OG1 | THR | C | 206 | 8.193 | −2.082 | −32.831 | 1.00 | 37.60 | O |
| ATOM | 10999 | CG2 | THR | C | 206 | 10.142 | −1.534 | −31.636 | 1.00 | 32.26 | C |
| ATOM | 11003 | C | THR | C | 206 | 8.850 | 1.164 | −30.953 | 1.00 | 33.16 | C |
| ATOM | 11004 | O | THR | C | 206 | 9.157 | 2.130 | −31.636 | 1.00 | 32.08 | O |
| ATOM | 11005 | N | PHE | C | 207 | 9.110 | 1.105 | −29.650 | 1.00 | 31.17 | N |
| ATOM | 11007 | CA | PHE | C | 207 | 9.812 | 2.164 | −28.960 | 1.00 | 30.14 | C |
| ATOM | 11009 | CB | PHE | C | 207 | 8.791 | 2.916 | −28.098 | 1.00 | 26.16 | C |
| ATOM | 11012 | CG | PHE | C | 207 | 7.677 | 3.532 | −28.865 | 1.00 | 26.96 | C |
| ATOM | 11013 | CD1 | PHE | C | 207 | 7.906 | 4.605 | −29.697 | 1.00 | 41.54 | C |
| ATOM | 11015 | CE1 | PHE | C | 207 | 6.868 | 5.176 | −30.421 | 1.00 | 31.22 | C |
| ATOM | 11017 | CZ | PHE | C | 207 | 5.583 | 4.691 | −30.299 | 1.00 | 36.10 | C |
| ATOM | 11019 | CE2 | PHE | C | 207 | 5.336 | 3.584 | −29.504 | 1.00 | 35.21 | C |
| ATOM | 11021 | CD2 | PHE | C | 207 | 6.385 | 3.018 | −28.781 | 1.00 | 29.64 | C |
| ATOM | 11023 | C | PHE | C | 207 | 10.965 | 1.628 | −28.060 | 1.00 | 29.67 | C |
| ATOM | 11024 | O | PHE | C | 207 | 10.888 | 0.557 | −27.455 | 1.00 | 30.12 | O |
| ATOM | 11025 | N | ALA | C | 208 | 12.030 | 2.397 | −27.893 | 1.00 | 27.61 | N |
| ATOM | 11027 | CA | ALA | C | 208 | 13.100 | 1.937 | −27.003 | 1.00 | 28.48 | C |
| ATOM | 11029 | CB | ALA | C | 208 | 14.340 | 2.764 | −27.233 | 1.00 | 23.20 | C |
| ATOM | 11033 | C | ALA | C | 208 | 12.685 | 1.979 | −25.508 | 1.00 | 24.90 | C |
| ATOM | 11034 | O | ALA | C | 208 | 12.348 | 3.018 | −24.976 | 1.00 | 30.52 | O |
| ATOM | 11035 | N | LEU | C | 209 | 12.701 | 0.837 | −24.830 | 1.00 | 30.10 | N |
| ATOM | 11037 | CA | LEU | C | 209 | 12.198 | 0.734 | −23.471 | 1.00 | 34.74 | C |
| ATOM | 11039 | CB | LEU | C | 209 | 12.267 | −0.711 | −22.969 | 1.00 | 32.73 | C |
| ATOM | 11042 | CG | LEU | C | 209 | 10.988 | −1.448 | −23.288 | 1.00 | 29.76 | C |
| ATOM | 11044 | CD1 | LEU | C | 209 | 11.209 | −2.923 | −23.345 | 1.00 | 28.22 | C |
| ATOM | 11048 | CD2 | LEU | C | 209 | 9.816 | −1.043 | −22.399 | 1.00 | 30.13 | C |
| ATOM | 11052 | C | LEU | C | 209 | 13.025 | 1.589 | −22.535 | 1.00 | 39.55 | C |
| ATOM | 11053 | O | LEU | C | 209 | 12.541 | 2.006 | −21.480 | 1.00 | 36.23 | O |
| ATOM | 11054 | N | ARG | C | 210 | 14.289 | 1.801 | −22.889 | 1.00 | 32.51 | N |
| ATOM | 11056 | CA | ARG | C | 210 | 15.203 | 2.493 | −21.993 | 1.00 | 29.49 | C |
| ATOM | 11058 | CB | ARG | C | 210 | 16.606 | 2.604 | −22.637 | 1.00 | 39.71 | C |
| ATOM | 11061 | CG | ARG | C | 210 | 17.534 | 3.615 | −21.948 | 1.00 | 30.30 | C |
| ATOM | 11064 | CD | ARG | C | 210 | 18.959 | 3.694 | −22.512 | 1.00 | 53.85 | C |
| ATOM | 11067 | NE | ARG | C | 210 | 19.566 | 4.998 | −22.228 | 1.00 | 48.80 | N |
| ATOM | 11069 | CZ | ARG | C | 210 | 20.401 | 5.219 | −21.227 | 1.00 | 51.26 | C |
| ATOM | 11070 | NH1 | ARG | C | 210 | 20.746 | 4.220 | −20.428 | 1.00 | 57.04 | N |
| ATOM | 11073 | NH2 | ARG | C | 210 | 20.887 | 6.433 | −21.029 | 1.00 | 57.60 | N |
| ATOM | 11076 | C | ARG | C | 210 | 14.671 | 3.888 | −21.796 | 1.00 | 29.20 | C |
| ATOM | 11077 | O | ARG | C | 210 | 14.748 | 4.452 | −20.714 | 1.00 | 28.34 | O |
| ATOM | 11078 | N | TYR | C | 211 | 14.217 | 4.500 | −22.884 | 1.00 | 30.39 | N |
| ATOM | 11080 | CA | TYR | C | 211 | 13.753 | 5.859 | −22.758 | 1.00 | 27.06 | C |
| ATOM | 11082 | CB | TYR | C | 211 | 13.869 | 6.575 | −24.101 | 1.00 | 32.98 | C |
| ATOM | 11085 | CG | TYR | C | 211 | 15.311 | 6.779 | −24.532 | 1.00 | 29.94 | C |
| ATOM | 11086 | CD1 | TYR | C | 211 | 15.979 | 5.802 | −25.270 | 1.00 | 38.57 | C |
| ATOM | 11088 | CE1 | TYR | C | 211 | 17.315 | 5.933 | −25.576 | 1.00 | 32.54 | C |
| ATOM | 11090 | CZ | TYR | C | 211 | 17.981 | 7.097 | −25.204 | 1.00 | 42.43 | C |
| ATOM | 11091 | OH | TYR | C | 211 | 19.309 | 7.257 | −25.501 | 1.00 | 54.79 | O |
| ATOM | 11093 | CE2 | TYR | C | 211 | 17.350 | 8.057 | −24.445 | 1.00 | 36.32 | C |
| ATOM | 11095 | CD2 | TYR | C | 211 | 16.049 | 7.876 | −24.079 | 1.00 | 37.13 | C |
| ATOM | 11097 | C | TYR | C | 211 | 12.357 | 5.985 | −22.040 | 1.00 | 27.31 | C |
| ATOM | 11098 | O | TYR | C | 211 | 12.054 | 6.959 | −21.341 | 1.00 | 30.97 | O |
| ATOM | 11099 | N | LEU | C | 212 | 11.509 | 4.998 | −22.213 | 1.00 | 22.86 | N |
| ATOM | 11101 | CA | LEU | C | 212 | 10.185 | 4.990 | −21.570 | 1.00 | 29.77 | C |
| ATOM | 11103 | CB | LEU | C | 212 | 9.339 | 3.831 | −22.087 | 1.00 | 33.27 | C |
| ATOM | 11106 | CG | LEU | C | 212 | 8.895 | 3.875 | −23.559 | 1.00 | 28.90 | C |
| ATOM | 11108 | CD1 | LEU | C | 212 | 8.158 | 2.626 | −23.881 | 1.00 | 24.77 | C |
| ATOM | 11112 | CD2 | LEU | C | 212 | 8.037 | 5.063 | −23.917 | 1.00 | 38.40 | C |
| ATOM | 11116 | C | LEU | C | 212 | 10.368 | 4.830 | −20.071 | 1.00 | 32.42 | C |
| ATOM | 11117 | O | LEU | C | 212 | 9.586 | 5.361 | −19.286 | 1.00 | 37.23 | O |
| ATOM | 11118 | N | ASN | C | 213 | 11.459 | 4.179 | −19.672 | 1.00 | 30.58 | N |
| ATOM | 11120 | CA | ASN | C | 213 | 11.706 | 3.951 | −18.274 | 1.00 | 27.47 | C |
| ATOM | 11122 | CB | ASN | C | 213 | 12.727 | 2.828 | −18.068 | 1.00 | 40.67 | C |
| ATOM | 11125 | CG | ASN | C | 213 | 12.115 | 1.470 | −18.147 | 1.00 | 33.47 | C |
| ATOM | 11126 | OD1 | ASN | C | 213 | 11.048 | 1.232 | −17.603 | 1.00 | 45.62 | O |
| ATOM | 11127 | ND2 | ASN | C | 213 | 12.793 | 0.554 | −18.810 | 1.00 | 57.41 | N |
| ATOM | 11130 | C | ASN | C | 213 | 12.214 | 5.218 | −17.633 | 1.00 | 36.69 | C |
| ATOM | 11131 | O | ASN | C | 213 | 11.943 | 5.412 | −16.440 | 1.00 | 35.81 | O |
| ATOM | 11132 | N | PHE | C | 214 | 12.921 | 6.071 | −18.391 | 1.00 | 31.05 | N |
| ATOM | 11134 | CA | PHE | C | 214 | 13.141 | 7.450 | −17.940 | 1.00 | 34.32 | C |
| ATOM | 11136 | CB | PHE | C | 214 | 14.082 | 8.208 | −18.877 | 1.00 | 35.48 | C |
| ATOM | 11139 | CG | PHE | C | 214 | 15.553 | 7.810 | −18.751 | 1.00 | 37.13 | C |
| ATOM | 11140 | CD1 | PHE | C | 214 | 16.227 | 7.898 | −17.556 | 1.00 | 44.77 | C |
| ATOM | 11142 | CE1 | PHE | C | 214 | 17.587 | 7.563 | −17.465 | 1.00 | 47.78 | C |
| ATOM | 11144 | CZ | PHE | C | 214 | 18.274 | 7.133 | −18.570 | 1.00 | 42.36 | C |
| ATOM | 11146 | CE2 | PHE | C | 214 | 17.615 | 7.056 | −19.773 | 1.00 | 39.35 | C |
| ATOM | 11148 | CD2 | PHE | C | 214 | 16.262 | 7.364 | −19.856 | 1.00 | 41.75 | C |
| ATOM | 11150 | C | PHE | C | 214 | 11.824 | 8.253 | −17.739 | 1.00 | 32.01 | C |
| ATOM | 11151 | O | PHE | C | 214 | 11.652 | 8.982 | −16.759 | 1.00 | 31.93 | O |
| ATOM | 11152 | N | PHE | C | 215 | 10.942 | 8.219 | −18.729 | 1.00 | 22.63 | N |
| ATOM | 11154 | CA | PHE | C | 215 | 9.707 | 8.984 | −18.668 | 1.00 | 27.56 | C |
| ATOM | 11156 | CB | PHE | C | 215 | 8.883 | 8.697 | −19.915 | 1.00 | 22.03 | C |
| ATOM | 11159 | CG | PHE | C | 215 | 9.610 | 8.976 | −21.207 | 1.00 | 32.87 | C |
| ATOM | 11160 | CD1 | PHE | C | 215 | 10.835 | 9.605 | −21.198 | 1.00 | 31.09 | C |
| ATOM | 11162 | CE1 | PHE | C | 215 | 11.491 | 9.891 | −22.374 | 1.00 | 28.86 | C |
| ATOM | 11164 | CZ | PHE | C | 215 | 10.952 | 9.529 | −23.595 | 1.00 | 23.83 | C |
| ATOM | 11166 | CE2 | PHE | C | 215 | 9.722 | 8.928 | −23.642 | 1.00 | 30.94 | C |
| ATOM | 11168 | CD2 | PHE | C | 215 | 9.054 | 8.634 | −22.435 | 1.00 | 29.68 | C |
| ATOM | 11170 | C | PHE | C | 215 | 8.874 | 8.685 | −17.387 | 1.00 | 27.80 | C |
| ATOM | 11171 | O | PHE | C | 215 | 8.390 | 9.607 | −16.767 | 1.00 | 28.58 | O |
| ATOM | 11172 | N | THR | C | 216 | 8.809 | 7.427 | −16.946 | 1.00 | 27.94 | N |
| ATOM | 11174 | CA | THR | C | 216 | 8.030 | 7.027 | −15.770 | 1.00 | 28.65 | C |
| ATOM | 11176 | CB | THR | C | 216 | 7.859 | 5.523 | −15.775 | 1.00 | 31.27 | C |
| ATOM | 11178 | OG1 | THR | C | 216 | 9.154 | 4.893 | −15.825 | 1.00 | 32.90 | O |
| ATOM | 11180 | CG2 | THR | C | 216 | 7.129 | 5.105 | −17.042 | 1.00 | 24.36 | C |
| ATOM | 11184 | C | THR | C | 216 | 8.517 | 7.500 | −14.383 | 1.00 | 29.72 | C |
| ATOM | 11185 | O | THR | C | 216 | 7.860 | 7.257 | −13.371 | 1.00 | 26.97 | O |
| ATOM | 11186 | N | LYS | C | 217 | 9.846 | 8.200 | −14.350 | 1.00 | 25.74 | N |
| ATOM | 11188 | CA | LYS | C | 217 | 10.120 | 8.893 | −13.165 | 1.00 | 24.60 | C |
| ATOM | 11190 | CB | LYS | C | 217 | 11.532 | 9.506 | −13.359 | 1.00 | 30.87 | C |
| ATOM | 11193 | CG | LYS | C | 217 | 12.659 | 8.547 | −13.743 | 1.00 | 31.92 | C |
| ATOM | 11196 | CD | LYS | C | 217 | 13.359 | 7.967 | −12.536 | 1.00 | 56.95 | C |
| ATOM | 11199 | CE | LYS | C | 217 | 14.605 | 7.190 | −12.961 | 1.00 | 60.83 | C |
| ATOM | 11202 | NZ | LYS | C | 217 | 15.397 | 8.007 | −13.931 | 1.00 | 66.66 | N |
| ATOM | 11206 | C | LYS | C | 217 | 9.180 | 10.052 | −12.842 | 1.00 | 24.91 | C |
| ATOM | 11207 | O | LYS | C | 217 | 9.286 | 10.648 | −11.780 | 1.00 | 26.66 | O |
| ATOM | 11208 | N | ALA | C | 218 | 8.320 | 10.431 | −13.781 | 1.00 | 29.70 | N |
| ATOM | 11210 | CA | ALA | C | 218 | 7.322 | 11.466 | −13.531 | 1.00 | 23.88 | C |
| ATOM | 11212 | CB | ALA | C | 218 | 6.960 | 12.104 | −14.829 | 1.00 | 14.87 | C |
| ATOM | 11216 | C | ALA | C | 218 | 6.038 | 10.943 | −12.841 | 1.00 | 17.68 | C |
| ATOM | 11217 | O | ALA | C | 218 | 5.046 | 11.665 | −12.784 | 1.00 | 27.78 | O |
| ATOM | 11218 | N | THR | C | 219 | 6.015 | 9.665 | −12.496 | 1.00 | 21.80 | N |
| ATOM | 11220 | CA | THR | C | 219 | 4.806 | 9.012 | −12.012 | 1.00 | 28.32 | C |
| ATOM | 11222 | CB | THR | C | 219 | 5.082 | 7.531 | −11.704 | 1.00 | 30.75 | C |
| ATOM | 11224 | OG1 | THR | C | 219 | 5.392 | 6.894 | −12.941 | 1.00 | 30.50 | O |
| ATOM | 11226 | CG2 | THR | C | 219 | 3.761 | 6.813 | −11.147 | 1.00 | 29.34 | C |
| ATOM | 11230 | C | THR | C | 219 | 4.236 | 9.721 | −10.774 | 1.00 | 25.32 | C |
| ATOM | 11231 | O | THR | C | 219 | 3.075 | 9.997 | −10.751 | 1.00 | 23.78 | O |
| ATOM | 11232 | N | PRO | C | 220 | 5.070 | 10.070 | −9.802 | 1.00 | 28.32 | N |
| ATOM | 11233 | CA | PRO | C | 220 | 4.621 | 10.786 | −8.598 | 1.00 | 25.89 | C |
| ATOM | 11235 | CB | PRO | C | 220 | 5.958 | 11.171 | −7.906 | 1.00 | 28.02 | C |
| ATOM | 11238 | CG | PRO | C | 220 | 6.978 | 10.047 | −8.356 | 1.00 | 23.57 | C |
| ATOM | 11241 | CD | PRO | C | 220 | 6.539 | 9.821 | −9.807 | 1.00 | 27.43 | C |
| ATOM | 11244 | C | PRO | C | 220 | 3.783 | 12.040 | −8.900 | 1.00 | 30.51 | C |
| ATOM | 11245 | O | PRO | C | 220 | 2.963 | 12.452 | −8.082 | 1.00 | 40.33 | O |
| ATOM | 11246 | N | LEU | C | 221 | 3.955 | 12.626 | −10.077 | 1.00 | 29.28 | N |
| ATOM | 11248 | CA | LEU | C | 221 | 3.306 | 13.890 | −10.422 | 1.00 | 28.42 | C |
| ATOM | 11250 | CB | LEU | C | 221 | 4.000 | 14.492 | −11.655 | 1.00 | 26.31 | C |
| ATOM | 11253 | CG | LEU | C | 221 | 5.299 | 15.299 | −11.415 | 1.00 | 34.96 | C |
| ATOM | 11255 | CD1 | LEU | C | 221 | 6.109 | 15.443 | −12.663 | 1.00 | 29.75 | C |
| ATOM | 11259 | CD2 | LEU | C | 221 | 5.114 | 16.683 | −10.814 | 1.00 | 31.33 | C |
| ATOM | 11263 | C | LEU | C | 221 | 1.800 | 13.725 | −10.708 | 1.00 | 28.76 | C |
| ATOM | 11264 | O | LEU | C | 221 | 1.020 | 14.668 | −10.620 | 1.00 | 38.30 | O |
| ATOM | 11265 | N | SER | C | 222 | 1.402 | 12.532 | −11.128 | 1.00 | 31.50 | N |
| ATOM | 11267 | CA | SER | C | 222 | 0.047 | 12.323 | −11.565 | 1.00 | 33.22 | C |
| ATOM | 11269 | CB | SER | C | 222 | −0.085 | 12.750 | −13.014 | 1.00 | 38.66 | C |
| ATOM | 11272 | OG | SER | C | 222 | −1.393 | 12.493 | −13.476 | 1.00 | 42.63 | O |
| ATOM | 11274 | C | SER | C | 222 | −0.360 | 10.855 | −11.418 | 1.00 | 43.43 | C |
| ATOM | 11275 | O | SER | C | 222 | 0.461 | 9.943 | −11.618 | 1.00 | 27.73 | O |
| ATOM | 11276 | N | SER | C | 223 | −1.645 | 10.632 | −11.116 | 1.00 | 29.62 | N |
| ATOM | 11278 | CA | SER | C | 223 | −2.199 | 9.274 | −11.084 | 1.00 | 32.62 | C |
| ATOM | 11280 | CB | SER | C | 223 | −3.487 | 9.265 | −10.222 | 1.00 | 37.78 | C |
| ATOM | 11283 | OG | SER | C | 223 | −3.098 | 9.092 | −8.879 | 1.00 | 51.33 | O |
| ATOM | 11285 | C | SER | C | 223 | −2.521 | 8.696 | −12.464 | 1.00 | 26.59 | C |
| ATOM | 11286 | O | SER | C | 223 | −2.731 | 7.505 | −12.587 | 1.00 | 26.15 | O |
| ATOM | 11287 | N | THR | C | 224 | −2.628 | 9.512 | −13.503 | 1.00 | 26.68 | N |
| ATOM | 11289 | CA | THR | C | 224 | −2.753 | 8.947 | −14.836 | 1.00 | 32.30 | C |
| ATOM | 11291 | CB | THR | C | 224 | −4.100 | 9.341 | −15.465 | 1.00 | 37.83 | C |
| ATOM | 11293 | OG1 | THR | C | 224 | −4.264 | 10.754 | −15.335 | 1.00 | 38.79 | O |
| ATOM | 11295 | CG2 | THR | C | 224 | −5.254 | 8.741 | −14.658 | 1.00 | 46.38 | C |
| ATOM | 11299 | C | THR | C | 224 | −1.684 | 9.397 | −15.797 | 1.00 | 23.90 | C |
| ATOM | 11300 | O | THR | C | 224 | −0.989 | 10.399 | −15.588 | 1.00 | 24.84 | O |
| ATOM | 11301 | N | VAL | C | 225 | −1.643 | 8.681 | −16.902 | 1.00 | 25.49 | N |
| ATOM | 11303 | CA | VAL | C | 225 | −0.792 | 9.055 | −18.010 | 1.00 | 28.39 | C |
| ATOM | 11305 | CB | VAL | C | 225 | 0.510 | 8.187 | −17.950 | 1.00 | 31.57 | C |
| ATOM | 11307 | CG1 | VAL | C | 225 | 0.185 | 6.672 | −18.073 | 1.00 | 31.17 | C |
| ATOM | 11311 | CG2 | VAL | C | 225 | 1.476 | 8.595 | −19.072 | 1.00 | 23.86 | C |
| ATOM | 11315 | C | VAL | C | 225 | −1.615 | 8.843 | −19.270 | 1.00 | 23.37 | C |
| ATOM | 11316 | O | VAL | C | 225 | −2.418 | 7.894 | −19.329 | 1.00 | 30.30 | O |
| ATOM | 11317 | N | THR | C | 226 | −1.406 | 9.689 | −20.274 | 1.00 | 28.68 | N |
| ATOM | 11319 | CA | THR | C | 226 | −1.905 | 9.407 | −21.621 | 1.00 | 37.85 | C |
| ATOM | 11321 | CB | THR | C | 226 | −2.907 | 10.535 | −22.053 | 1.00 | 34.42 | C |
| ATOM | 11323 | OG1 | THR | C | 226 | −2.196 | 11.733 | −22.361 | 1.00 | 51.67 | O |
| ATOM | 11325 | CG2 | THR | C | 226 | −3.738 | 10.979 | −20.853 | 1.00 | 44.80 | C |
| ATOM | 11329 | C | THR | C | 226 | −0.785 | 9.191 | −22.678 | 1.00 | 39.35 | C |
| ATOM | 11330 | O | THR | C | 226 | 0.149 | 10.003 | −22.815 | 1.00 | 38.73 | O |
| ATOM | 11331 | N | LEU | C | 227 | −0.934 | 8.106 | −23.440 | 1.00 | 32.94 | N |
| ATOM | 11333 | CA | LEU | C | 227 | −0.137 | 7.839 | −24.614 | 1.00 | 29.82 | C |
| ATOM | 11335 | CB | LEU | C | 227 | 0.383 | 6.402 | −24.542 | 1.00 | 29.51 | C |
| ATOM | 11338 | CG | LEU | C | 227 | 0.676 | 5.920 | −23.115 | 1.00 | 30.84 | C |
| ATOM | 11340 | CD1 | LEU | C | 227 | 0.648 | 4.399 | −23.074 | 1.00 | 37.57 | C |
| ATOM | 11344 | CD2 | LEU | C | 227 | 1.986 | 6.444 | −22.614 | 1.00 | 24.85 | C |
| ATOM | 11348 | C | LEU | C | 227 | −0.881 | 8.144 | −25.929 | 1.00 | 34.93 | C |
| ATOM | 11349 | O | LEU | C | 227 | −1.904 | 7.507 | −26.260 | 1.00 | 32.42 | O |
| ATOM | 11350 | N | SER | C | 228 | −0.340 | 9.127 | −26.664 | 1.00 | 30.23 | N |
| ATOM | 11352 | CA | SER | C | 228 | −0.807 | 9.531 | −27.990 | 1.00 | 27.03 | C |
| ATOM | 11354 | CB | SER | C | 228 | −0.850 | 11.044 | −28.036 | 1.00 | 30.40 | C |
| ATOM | 11357 | OG | SER | C | 228 | −1.642 | 11.478 | −26.947 | 1.00 | 39.29 | O |
| ATOM | 11359 | C | SER | C | 228 | 0.144 | 9.040 | −29.063 | 1.00 | 36.17 | C |
| ATOM | 11360 | O | SER | C | 228 | 1.354 | 9.342 | −29.046 | 1.00 | 25.04 | O |
| ATOM | 11361 | N | MET | C | 229 | −0.415 | 8.343 | −30.039 | 1.00 | 35.68 | N |
| ATOM | 11363 | CA | MET | C | 229 | 0.400 | 7.677 | −31.045 | 1.00 | 33.87 | C |
| ATOM | 11365 | CB | MET | C | 229 | 0.434 | 6.197 | −30.740 | 1.00 | 35.55 | C |
| ATOM | 11368 | CG | MET | C | 229 | 1.416 | 5.797 | −29.708 | 1.00 | 49.94 | C |
| ATOM | 11371 | SD | MET | C | 229 | 1.044 | 4.133 | −29.187 | 1.00 | 49.38 | S |
| ATOM | 11372 | CE | MET | C | 229 | −0.564 | 4.372 | −28.288 | 1.00 | 49.39 | C |
| ATOM | 11376 | C | MET | C | 229 | −0.218 | 7.831 | −32.415 | 1.00 | 39.71 | C |
| ATOM | 11377 | O | MET | C | 229 | −1.422 | 7.637 | −32.550 | 1.00 | 33.70 | O |
| ATOM | 11378 | N | SER | C | 230 | 0.604 | 8.030 | −33.451 | 1.00 | 47.00 | N |
| ATOM | 11380 | CA | SER | C | 230 | 0.202 | 7.609 | −34.804 | 1.00 | 48.31 | C |
| ATOM | 11382 | CB | SER | C | 230 | −0.481 | 8.718 | −35.619 | 1.00 | 46.20 | C |
| ATOM | 11385 | OG | SER | C | 230 | −0.827 | 9.846 | −34.862 | 1.00 | 48.79 | O |
| ATOM | 11387 | C | SER | C | 230 | 1.353 | 7.049 | −35.606 | 1.00 | 41.77 | C |
| ATOM | 11388 | O | SER | C | 230 | 2.497 | 7.146 | −35.184 | 1.00 | 51.15 | O |
| ATOM | 11389 | N | ALA | C | 231 | 1.071 | 6.482 | −36.773 | 1.00 | 42.87 | N |
| ATOM | 11391 | CA | ALA | C | 231 | 2.150 | 5.871 | −37.557 | 1.00 | 48.60 | C |
| ATOM | 11393 | CB | ALA | C | 231 | 1.604 | 5.232 | −38.831 | 1.00 | 48.71 | C |
| ATOM | 11397 | C | ALA | C | 231 | 3.278 | 6.876 | −37.864 | 1.00 | 47.26 | C |
| ATOM | 11398 | O | ALA | C | 231 | 3.026 | 8.082 | −38.090 | 1.00 | 47.85 | O |
| ATOM | 11399 | N | ASP | C | 232 | 4.512 | 6.394 | −37.697 | 1.00 | 51.17 | N |
| ATOM | 11401 | CA | ASP | C | 232 | 5.746 | 7.109 | −38.063 | 1.00 | 54.78 | C |
| ATOM | 11403 | CB | ASP | C | 232 | 5.813 | 7.306 | −39.578 | 1.00 | 60.72 | C |
| ATOM | 11406 | CG | ASP | C | 232 | 5.763 | 5.982 | −40.336 | 1.00 | 73.12 | C |
| ATOM | 11407 | OD1 | ASP | C | 232 | 5.248 | 5.964 | −41.478 | 1.00 | 82.44 | O |
| ATOM | 11408 | OD2 | ASP | C | 232 | 6.200 | 4.910 | −39.858 | 1.00 | 88.84 | O |
| ATOM | 11409 | C | ASP | C | 232 | 6.074 | 8.432 | −37.358 | 1.00 | 53.37 | C |
| ATOM | 11410 | O | ASP | C | 232 | 7.145 | 9.002 | −37.600 | 1.00 | 53.05 | O |
| ATOM | 11411 | N | VAL | C | 233 | 5.208 | 8.889 | −36.446 | 1.00 | 56.94 | N |
| ATOM | 11413 | CA | VAL | C | 233 | 5.477 | 10.097 | −35.635 | 1.00 | 44.20 | C |
| ATOM | 11415 | CB | VAL | C | 233 | 4.353 | 11.139 | −35.861 | 1.00 | 47.08 | C |
| ATOM | 11417 | CG1 | VAL | C | 233 | 3.887 | 11.053 | −37.333 | 1.00 | 43.74 | C |
| ATOM | 11421 | CG2 | VAL | C | 233 | 3.153 | 10.925 | −34.921 | 1.00 | 48.19 | C |
| ATOM | 11425 | C | VAL | C | 233 | 5.784 | 9.785 | −34.139 | 1.00 | 41.29 | C |
| ATOM | 11426 | O | VAL | C | 233 | 5.566 | 8.657 | −33.648 | 1.00 | 39.35 | O |
| ATOM | 11427 | N | PRO | C | 234 | 6.404 | 10.729 | −33.441 | 1.00 | 26.45 | N |
| ATOM | 11428 | CA | PRO | C | 234 | 6.737 | 10.506 | −32.032 | 1.00 | 32.01 | C |
| ATOM | 11430 | CB | PRO | C | 234 | 7.513 | 11.771 | −31.633 | 1.00 | 36.99 | C |
| ATOM | 11433 | CG | PRO | C | 234 | 8.057 | 12.312 | −32.934 | 1.00 | 32.37 | C |
| ATOM | 11436 | CD | PRO | C | 234 | 6.884 | 12.043 | −33.910 | 1.00 | 40.54 | C |
| ATOM | 11439 | C | PRO | C | 234 | 5.496 | 10.263 | −31.141 | 1.00 | 41.44 | C |
| ATOM | 11440 | O | PRO | C | 234 | 4.448 | 10.938 | −31.198 | 1.00 | 28.90 | O |
| ATOM | 11441 | N | LEU | C | 235 | 5.662 | 9.232 | −30.325 | 1.00 | 31.59 | N |
| ATOM | 11443 | CA | LEU | C | 235 | 4.886 | 9.018 | −29.129 | 1.00 | 30.38 | C |
| ATOM | 11445 | CB | LEU | C | 235 | 5.430 | 7.774 | −28.419 | 1.00 | 22.64 | C |
| ATOM | 11448 | CG | LEU | C | 235 | 4.911 | 7.489 | −27.012 | 1.00 | 26.83 | C |
| ATOM | 11450 | CD1 | LEU | C | 235 | 3.350 | 7.272 | −26.993 | 1.00 | 26.15 | C |
| ATOM | 11454 | CD2 | LEU | C | 235 | 5.660 | 6.274 | −26.461 | 1.00 | 25.38 | C |
| ATOM | 11458 | C | LEU | C | 235 | 5.002 | 10.219 | −28.217 | 1.00 | 35.15 | C |
| ATOM | 11459 | O | LEU | C | 235 | 6.116 | 10.682 | −27.963 | 1.00 | 28.82 | O |
| ATOM | 11460 | N | VAL | C | 236 | 3.866 | 10.623 | −27.640 | 1.00 | 27.02 | N |
| ATOM | 11462 | CA | VAL | C | 236 | 3.829 | 11.529 | −26.503 | 1.00 | 27.97 | C |
| ATOM | 11464 | CB | VAL | C | 236 | 2.884 | 12.695 | −26.787 | 1.00 | 28.69 | C |
| ATOM | 11466 | CG1 | VAL | C | 236 | 3.095 | 13.820 | −25.875 | 1.00 | 26.46 | C |
| ATOM | 11470 | CG2 | VAL | C | 236 | 3.076 | 13.160 | −28.174 | 1.00 | 37.57 | C |
| ATOM | 11474 | C | VAL | C | 236 | 3.329 | 10.814 | −25.256 | 1.00 | 34.24 | C |
| ATOM | 11475 | O | VAL | C | 236 | 2.259 | 10.175 | −25.282 | 1.00 | 28.84 | O |
| ATOM | 11476 | N | VAL | C | 237 | 4.135 | 10.884 | −24.192 | 1.00 | 26.12 | N |
| ATOM | 11478 | CA | VAL | C | 237 | 3.755 | 10.426 | −22.866 | 1.00 | 23.04 | C |
| ATOM | 11480 | CB | VAL | C | 237 | 4.835 | 9.563 | −22.262 | 1.00 | 26.22 | C |
| ATOM | 11482 | CG1 | VAL | C | 237 | 4.316 | 8.910 | −21.031 | 1.00 | 30.16 | C |
| ATOM | 11486 | CG2 | VAL | C | 237 | 5.255 | 8.512 | −23.253 | 1.00 | 21.81 | C |
| ATOM | 11490 | C | VAL | C | 237 | 3.532 | 11.679 | −22.040 | 1.00 | 30.98 | C |
| ATOM | 11491 | O | VAL | C | 237 | 4.439 | 12.505 | −21.961 | 1.00 | 28.43 | O |
| ATOM | 11492 | N | GLU | C | 238 | 2.294 | 11.875 | −21.556 | 1.00 | 27.40 | N |
| ATOM | 11494 | CA | GLU | C | 238 | 1.886 | 13.125 | −20.924 | 1.00 | 24.98 | C |
| ATOM | 11496 | CB | GLU | C | 238 | 0.674 | 13.678 | −21.659 | 1.00 | 32.86 | C |
| ATOM | 11499 | CG | GLU | C | 238 | 0.333 | 15.139 | −21.365 | 1.00 | 47.45 | C |
| ATOM | 11502 | CD | GLU | C | 238 | −0.856 | 15.589 | −22.196 | 1.00 | 58.86 | C |
| ATOM | 11503 | OE1 | GLU | C | 238 | −0.839 | 15.330 | −23.428 | 1.00 | 63.99 | O |
| ATOM | 11504 | OE2 | GLU | C | 238 | −1.818 | 16.140 | −21.615 | 1.00 | 78.02 | O |
| ATOM | 11505 | C | GLU | C | 238 | 1.510 | 12.908 | −19.470 | 1.00 | 19.73 | C |
| ATOM | 11506 | O | GLU | C | 238 | 0.678 | 12.060 | −19.157 | 1.00 | 25.51 | O |
| ATOM | 11507 | N | TYR | C | 239 | 2.125 | 13.672 | −18.573 | 1.00 | 20.23 | N |
| ATOM | 11509 | CA | TYR | C | 239 | 1.731 | 13.721 | −17.189 | 1.00 | 26.64 | C |
| ATOM | 11511 | CB | TYR | C | 239 | 2.890 | 13.394 | −16.284 | 1.00 | 25.91 | C |
| ATOM | 11514 | CG | TYR | C | 239 | 3.458 | 12.043 | −16.540 | 1.00 | 28.28 | C |
| ATOM | 11515 | CD1 | TYR | C | 239 | 4.472 | 11.855 | −17.492 | 1.00 | 29.48 | C |
| ATOM | 11517 | CE1 | TYR | C | 239 | 4.969 | 10.596 | −17.733 | 1.00 | 24.40 | C |
| ATOM | 11519 | CZ | TYR | C | 239 | 4.482 | 9.522 | −16.985 | 1.00 | 24.53 | C |
| ATOM | 11520 | OH | TYR | C | 239 | 4.976 | 8.253 | −17.172 | 1.00 | 28.69 | O |
| ATOM | 11522 | CE2 | TYR | C | 239 | 3.519 | 9.704 | −16.019 | 1.00 | 31.55 | C |
| ATOM | 11524 | CD2 | TYR | C | 239 | 3.002 | 10.951 | −15.814 | 1.00 | 41.67 | C |
| ATOM | 11526 | C | TYR | C | 239 | 1.223 | 15.115 | −16.859 | 1.00 | 30.60 | C |
| ATOM | 11527 | O | TYR | C | 239 | 1.955 | 16.086 | −16.977 | 1.00 | 25.83 | O |
| ATOM | 11528 | N | LYS | C | 240 | 0.005 | 15.165 | −16.322 | 1.00 | 30.41 | N |
| ATOM | 11530 | CA | LYS | C | 240 | −0.570 | 16.379 | −15.769 | 1.00 | 33.85 | C |
| ATOM | 11532 | CB | LYS | C | 240 | −2.083 | 16.258 | −15.747 | 1.00 | 36.99 | C |
| ATOM | 11535 | CG | LYS | C | 240 | −2.787 | 16.956 | −16.883 | 1.00 | 55.11 | C |
| ATOM | 11538 | CD | LYS | C | 240 | −2.277 | 16.569 | −18.277 | 1.00 | 75.76 | C |
| ATOM | 11541 | CE | LYS | C | 240 | −1.755 | 17.780 | −19.076 | 1.00 | 78.08 | C |
| ATOM | 11544 | NZ | LYS | C | 240 | −2.818 | 18.677 | −19.637 | 1.00 | 90.66 | N |
| ATOM | 11548 | C | LYS | C | 240 | 0.004 | 16.737 | −14.396 | 1.00 | 33.86 | C |
| ATOM | 11549 | O | LYS | C | 240 | 0.123 | 15.904 | −13.500 | 1.00 | 40.68 | O |
| ATOM | 11550 | N | ILE | C | 241 | 0.439 | 17.993 | −14.289 | 1.00 | 30.16 | N |
| ATOM | 11552 | CA | ILE | C | 241 | 0.976 | 18.558 | −13.066 | 1.00 | 29.04 | C |
| ATOM | 11554 | CB | ILE | C | 241 | 2.180 | 19.406 | −13.407 | 1.00 | 32.54 | C |
| ATOM | 11556 | CG1 | ILE | C | 241 | 3.283 | 18.537 | −14.041 | 1.00 | 31.87 | C |
| ATOM | 11559 | CD1 | ILE | C | 241 | 4.491 | 19.403 | −14.482 | 1.00 | 30.30 | C |
| ATOM | 11563 | CG2 | ILE | C | 241 | 2.739 | 20.121 | −12.181 | 1.00 | 39.56 | C |
| ATOM | 11567 | C | ILE | C | 241 | −0.153 | 19.344 | −12.414 | 1.00 | 37.37 | C |
| ATOM | 11568 | O | ILE | C | 241 | −0.381 | 20.503 | −12.737 | 1.00 | 42.56 | O |
| ATOM | 11569 | N | ALA | C | 242 | −1.007 | 18.608 | −11.707 | 1.00 | 48.87 | N |
| ATOM | 11571 | CA | ALA | C | 242 | −1.852 | 19.132 | −10.635 | 1.00 | 43.05 | C |
| ATOM | 11573 | CB | ALA | C | 242 | −1.074 | 19.251 | −9.361 | 1.00 | 50.41 | C |
| ATOM | 11577 | C | ALA | C | 242 | −2.439 | 20.466 | −10.976 | 1.00 | 37.96 | C |
| ATOM | 11578 | O | ALA | C | 242 | −2.271 | 21.415 | −10.234 | 1.00 | 55.71 | O |
| ATOM | 11579 | N | ASP | C | 243 | −3.070 | 20.533 | −12.133 | 1.00 | 39.25 | N |
| ATOM | 11581 | CA | ASP | C | 243 | −3.756 | 21.726 | −12.592 | 1.00 | 45.16 | C |
| ATOM | 11583 | CB | ASP | C | 243 | −4.889 | 22.022 | −11.624 | 1.00 | 48.13 | C |
| ATOM | 11586 | CG | ASP | C | 243 | −5.917 | 20.911 | −11.653 | 1.00 | 60.42 | C |
| ATOM | 11587 | OD1 | ASP | C | 243 | −6.535 | 20.715 | −12.724 | 1.00 | 72.71 | O |
| ATOM | 11588 | OD2 | ASP | C | 243 | −6.006 | 20.047 | −10.756 | 1.00 | 82.99 | O |
| ATOM | 11589 | C | ASP | C | 243 | −2.943 | 22.955 | −13.030 | 1.00 | 44.94 | C |
| ATOM | 11590 | O | ASP | C | 243 | −3.524 | 23.926 | −13.492 | 1.00 | 48.97 | O |
| ATOM | 11591 | N | MET | C | 244 | −1.614 | 22.840 | −12.997 | 1.00 | 44.88 | N |
| ATOM | 11593 | CA | MET | C | 244 | −0.632 | 23.917 | −13.231 | 1.00 | 38.79 | C |
| ATOM | 11595 | CB | MET | C | 244 | 0.552 | 23.634 | −12.321 | 1.00 | 43.66 | C |
| ATOM | 11598 | CG | MET | C | 244 | 0.475 | 24.148 | −10.939 | 1.00 | 47.64 | C |
| ATOM | 11601 | SD | MET | C | 244 | 2.051 | 24.967 | −10.697 | 1.00 | 60.86 | S |
| ATOM | 11602 | CE | MET | C | 244 | 3.180 | 23.634 | −10.853 | 1.00 | 74.99 | C |
| ATOM | 11606 | C | MET | C | 244 | −0.042 | 23.831 | −14.658 | 1.00 | 32.60 | C |
| ATOM | 11607 | O | MET | C | 244 | 0.450 | 24.811 | −15.170 | 1.00 | 38.27 | O |
| ATOM | 11608 | N | GLY | C | 245 | −0.072 | 22.637 | −15.266 | 1.00 | 32.05 | N |
| ATOM | 11610 | CA | GLY | C | 245 | 0.453 | 22.387 | −16.586 | 1.00 | 29.63 | C |
| ATOM | 11613 | C | GLY | C | 245 | 0.817 | 20.920 | −16.745 | 1.00 | 35.16 | C |
| ATOM | 11614 | O | GLY | C | 245 | −0.030 | 20.077 | −16.484 | 1.00 | 23.77 | O |
| ATOM | 11615 | N | HIS | C | 246 | 2.004 | 20.623 | −17.290 | 1.00 | 25.47 | N |
| ATOM | 11617 | CA | HIS | C | 246 | 2.282 | 19.310 | −17.842 | 1.00 | 32.55 | C |
| ATOM | 11619 | CB | HIS | C | 246 | 1.438 | 19.054 | −19.093 | 1.00 | 31.50 | C |
| ATOM | 11622 | CG | HIS | C | 246 | 1.759 | 19.947 | −20.242 | 1.00 | 52.21 | C |
| ATOM | 11623 | ND1 | HIS | C | 246 | 0.802 | 20.710 | −20.874 | 1.00 | 51.17 | N |
| ATOM | 11625 | CE1 | HIS | C | 246 | 1.369 | 21.374 | −21.869 | 1.00 | 53.13 | C |
| ATOM | 11627 | NE2 | HIS | C | 246 | 2.657 | 21.082 | −21.892 | 1.00 | 57.61 | N |
| ATOM | 11629 | CD2 | HIS | C | 246 | 2.924 | 20.173 | −20.897 | 1.00 | 59.16 | C |
| ATOM | 11631 | C | HIS | C | 246 | 3.748 | 19.010 | −18.152 | 1.00 | 28.94 | C |
| ATOM | 11632 | O | HIS | C | 246 | 4.602 | 19.898 | −18.212 | 1.00 | 29.08 | O |
| ATOM | 11633 | N | LEU | C | 247 | 4.015 | 17.722 | −18.284 | 1.00 | 28.60 | N |
| ATOM | 11635 | CA | LEU | C | 247 | 5.316 | 17.225 | −18.696 | 1.00 | 31.23 | C |
| ATOM | 11637 | CB | LEU | C | 247 | 6.066 | 16.705 | −17.489 | 1.00 | 27.63 | C |
| ATOM | 11640 | CG | LEU | C | 247 | 7.439 | 16.067 | −17.643 | 1.00 | 34.81 | C |
| ATOM | 11642 | CD1 | LEU | C | 247 | 8.443 | 17.132 | −18.150 | 1.00 | 47.69 | C |
| ATOM | 11646 | CD2 | LEU | C | 247 | 7.892 | 15.539 | −16.285 | 1.00 | 46.77 | C |
| ATOM | 11650 | C | LEU | C | 247 | 5.035 | 16.151 | −19.749 | 1.00 | 22.96 | C |
| ATOM | 11651 | O | LEU | C | 247 | 4.509 | 15.097 | −19.471 | 1.00 | 30.91 | O |
| ATOM | 11652 | N | LYS | C | 248 | 5.298 | 16.535 | −20.992 | 1.00 | 24.83 | N |
| ATOM | 11654 | CA | LYS | C | 248 | 5.337 | 15.655 | −22.147 | 1.00 | 33.17 | C |
| ATOM | 11656 | CB | LYS | C | 248 | 4.753 | 16.430 | −23.331 | 1.00 | 36.51 | C |
| ATOM | 11659 | CG | LYS | C | 248 | 3.329 | 16.938 | −23.023 | 1.00 | 30.66 | C |
| ATOM | 11662 | CD | LYS | C | 248 | 2.660 | 17.622 | −24.192 | 1.00 | 47.03 | C |
| ATOM | 11665 | CE | LYS | C | 248 | 1.268 | 18.150 | −23.760 | 1.00 | 47.41 | C |
| ATOM | 11668 | NZ | LYS | C | 248 | 0.531 | 18.860 | −24.843 | 1.00 | 51.54 | N |
| ATOM | 11672 | C | LYS | C | 248 | 6.759 | 15.196 | −22.505 | 1.00 | 35.75 | C |
| ATOM | 11673 | O | LYS | C | 248 | 7.720 | 15.972 | −22.576 | 1.00 | 30.48 | O |
| ATOM | 11674 | N | TYR | C | 249 | 6.825 | 13.930 | −22.855 | 1.00 | 31.77 | N |
| ATOM | 11676 | CA | TYR | C | 249 | 8.033 | 13.329 | −23.332 | 1.00 | 32.10 | C |
| ATOM | 11678 | CB | TYR | C | 249 | 8.339 | 12.170 | −22.403 | 1.00 | 30.10 | C |
| ATOM | 11681 | CG | TYR | C | 249 | 8.633 | 12.521 | −20.979 | 1.00 | 27.14 | C |
| ATOM | 11682 | CD1 | TYR | C | 249 | 9.891 | 12.919 | −20.640 | 1.00 | 20.63 | C |
| ATOM | 11684 | CE1 | TYR | C | 249 | 10.244 | 13.126 | −19.343 | 1.00 | 18.24 | C |
| ATOM | 11686 | CZ | TYR | C | 249 | 9.326 | 12.984 | −18.328 | 1.00 | 28.36 | C |
| ATOM | 11687 | OH | TYR | C | 249 | 9.749 | 13.244 | −17.050 | 1.00 | 33.06 | O |
| ATOM | 11689 | CE2 | TYR | C | 249 | 8.013 | 12.635 | −18.627 | 1.00 | 20.74 | C |
| ATOM | 11691 | CD2 | TYR | C | 249 | 7.671 | 12.403 | −19.946 | 1.00 | 22.63 | C |
| ATOM | 11693 | C | TYR | C | 249 | 7.743 | 12.772 | −24.740 | 1.00 | 35.49 | C |
| ATOM | 11694 | O | TYR | C | 249 | 6.837 | 11.950 | −24.900 | 1.00 | 33.15 | O |
| ATOM | 11695 | N | TYR | C | 250 | 8.518 | 13.200 | −25.739 | 1.00 | 28.05 | N |
| ATOM | 11697 | CA | TYR | C | 250 | 8.360 | 12.735 | −27.121 | 1.00 | 24.82 | C |
| ATOM | 11699 | CB | TYR | C | 250 | 8.476 | 13.944 | −28.051 | 1.00 | 24.91 | C |
| ATOM | 11702 | CG | TYR | C | 250 | 7.437 | 15.032 | −27.798 | 1.00 | 24.71 | C |
| ATOM | 11703 | CD1 | TYR | C | 250 | 7.594 | 15.969 | −26.774 | 1.00 | 25.42 | C |
| ATOM | 11705 | CE1 | TYR | C | 250 | 6.634 | 16.932 | −26.540 | 1.00 | 36.27 | C |
| ATOM | 11707 | CZ | TYR | C | 250 | 5.477 | 16.944 | −27.315 | 1.00 | 27.23 | C |
| ATOM | 11708 | OH | TYR | C | 250 | 4.541 | 17.929 | −27.160 | 1.00 | 43.06 | O |
| ATOM | 11710 | CE2 | TYR | C | 250 | 5.314 | 16.055 | −28.352 | 1.00 | 25.41 | C |
| ATOM | 11712 | CD2 | TYR | C | 250 | 6.303 | 15.116 | −28.598 | 1.00 | 24.32 | C |
| ATOM | 11714 | C | TYR | C | 250 | 9.447 | 11.739 | −27.486 | 1.00 | 23.84 | C |
| ATOM | 11715 | O | TYR | C | 250 | 10.619 | 11.956 | −27.205 | 1.00 | 31.46 | O |
| ATOM | 11716 | N | LEU | C | 251 | 9.054 | 10.696 | −28.201 | 1.00 | 20.66 | N |
| ATOM | 11718 | CA | LEU | C | 251 | 9.929 | 9.629 | −28.606 | 1.00 | 28.61 | C |
| ATOM | 11720 | CB | LEU | C | 251 | 9.905 | 8.510 | −27.572 | 1.00 | 30.73 | C |
| ATOM | 11723 | CG | LEU | C | 251 | 10.888 | 7.369 | −27.769 | 1.00 | 27.62 | C |
| ATOM | 11725 | CD1 | LEU | C | 251 | 12.389 | 7.832 | −27.751 | 1.00 | 37.09 | C |
| ATOM | 11729 | CD2 | LEU | C | 251 | 10.626 | 6.306 | −26.729 | 1.00 | 26.63 | C |
| ATOM | 11733 | C | LEU | C | 251 | 9.552 | 9.085 | −29.981 | 1.00 | 32.82 | C |
| ATOM | 11734 | O | LEU | C | 251 | 8.479 | 8.464 | −30.155 | 1.00 | 27.27 | O |
| ATOM | 11735 | N | ALA | C | 252 | 10.524 | 9.140 | −30.899 | 1.00 | 36.36 | N |
| ATOM | 11737 | CA | ALA | C | 252 | 10.412 | 8.458 | −32.197 | 1.00 | 28.72 | C |
| ATOM | 11739 | CB | ALA | C | 252 | 11.679 | 8.739 | −33.087 | 1.00 | 28.01 | C |
| ATOM | 11743 | C | ALA | C | 252 | 10.196 | 6.958 | −32.045 | 1.00 | 23.39 | C |
| ATOM | 11744 | O | ALA | C | 252 | 10.939 | 6.258 | −31.385 | 1.00 | 23.82 | O |
| ATOM | 11745 | N | PRO | C | 253 | 9.284 | 6.408 | −32.826 | 1.00 | 27.34 | N |
| ATOM | 11746 | CA | PRO | C | 253 | 9.251 | 4.954 | −33.000 | 1.00 | 34.23 | C |
| ATOM | 11748 | CB | PRO | C | 253 | 7.939 | 4.703 | −33.752 | 1.00 | 27.89 | C |
| ATOM | 11751 | CG | PRO | C | 253 | 7.388 | 6.045 | −34.078 | 1.00 | 30.95 | C |
| ATOM | 11754 | CD | PRO | C | 253 | 8.383 | 7.096 | −33.755 | 1.00 | 32.29 | C |
| ATOM | 11757 | C | PRO | C | 253 | 10.377 | 4.486 | −33.889 | 1.00 | 32.09 | C |
| ATOM | 11758 | O | PRO | C | 253 | 10.940 | 5.290 | −34.641 | 1.00 | 41.22 | O |
| ATOM | 11759 | N | LYS | C | 254 | 10.655 | 3.194 | −33.820 | 1.00 | 37.24 | N |
| ATOM | 11761 | CA | LYS | C | 254 | 11.575 | 2.549 | −34.739 | 1.00 | 46.57 | C |
| ATOM | 11763 | CB | LYS | C | 254 | 11.935 | 1.179 | −34.206 | 1.00 | 40.56 | C |
| ATOM | 11766 | CG | LYS | C | 254 | 12.956 | 1.257 | −33.086 | 1.00 | 55.28 | C |
| ATOM | 11769 | CD | LYS | C | 254 | 13.329 | −0.141 | −32.562 | 1.00 | 60.03 | C |
| ATOM | 11772 | CE | LYS | C | 254 | 14.044 | −0.073 | −31.199 | 1.00 | 55.15 | C |
| ATOM | 11775 | NZ | LYS | C | 254 | 14.965 | 1.093 | −31.041 | 1.00 | 47.18 | N |
| ATOM | 11779 | C | LYS | C | 254 | 10.975 | 2.390 | −36.128 | 1.00 | 48.96 | C |
| ATOM | 11780 | O | LYS | C | 254 | 10.246 | 1.432 | −36.318 | 1.00 | 52.12 | O |
| ATOM | 11781 | N | ILE | C | 255 | 11.337 | 3.287 | −37.059 | 1.00 | 50.26 | N |
| ATOM | 11783 | CA | ILE | C | 255 | 10.750 | 3.447 | −38.395 | 1.00 | 56.20 | C |
| ATOM | 11785 | CB | ILE | C | 255 | 10.415 | 2.107 | −39.059 | 1.00 | 60.23 | C |
| ATOM | 11787 | CG1 | ILE | C | 255 | 11.710 | 1.376 | −39.412 | 1.00 | 54.93 | C |
| ATOM | 11790 | CD1 | ILE | C | 255 | 11.694 | −0.098 | −39.079 | 1.00 | 62.63 | C |
| ATOM | 11794 | CG2 | ILE | C | 255 | 9.560 | 2.337 | −40.311 | 1.00 | 69.84 | C |
| ATOM | 11798 | C | ILE | C | 255 | 9.533 | 4.364 | −38.411 | 1.00 | 63.00 | C |
| ATOM | 11799 | O | ILE | C | 255 | 9.642 | 5.548 | −38.079 | 1.00 | 68.30 | O |
| ATOM | 11800 | OW0 | HOH | W | 1 | 17.051 | 23.557 | 3.874 | 1.00 | 24.17 | O |
| ATOM | 11803 | OW0 | HOH | W | 2 | 9.152 | 12.787 | −10.016 | 1.00 | 25.85 | O |
| ATOM | 11806 | OW0 | HOH | W | 3 | 3.489 | 7.742 | −32.081 | 1.00 | 38.42 | O |
| ATOM | 11809 | OW0 | HOH | W | 4 | 15.980 | 0.448 | −24.763 | 1.00 | 40.48 | O |
| ATOM | 11812 | OW0 | HOH | W | 5 | 16.505 | −0.521 | 31.459 | 1.00 | 60.06 | O |
| ATOM | 11815 | OW0 | HOH | W | 6 | 16.017 | −20.487 | 7.996 | 1.00 | 23.91 | O |
| ATOM | 11818 | OW0 | HOH | W | 7 | 19.076 | −17.617 | −3.768 | 1.00 | 47.85 | O |
| ATOM | 11821 | OW0 | HOH | W | 8 | 20.684 | 15.391 | 15.881 | 1.00 | 30.68 | O |
| ATOM | 11824 | OW0 | HOH | W | 9 | 5.925 | 36.910 | −6.880 | 1.00 | 39.00 | O |
| ATOM | 11827 | OW0 | HOH | W | 10 | 32.609 | 26.656 | 20.575 | 1.00 | 47.96 | O |
| ATOM | 11830 | OW0 | HOH | W | 11 | −1.509 | 15.832 | −11.067 | 1.00 | 42.04 | O |
| ATOM | 11833 | OW0 | HOH | W | 12 | 8.859 | 30.517 | −0.036 | 1.00 | 29.92 | O |
| ATOM | 11836 | OW0 | HOH | W | 13 | 35.635 | 21.898 | 12.510 | 1.00 | 30.86 | O |
| ATOM | 11839 | OW0 | HOH | W | 14 | −0.325 | −24.722 | −4.359 | 1.00 | 54.55 | O |
| ATOM | 11842 | OW0 | HOH | W | 15 | 5.113 | −24.579 | 4.231 | 1.00 | 31.64 | O |
| ATOM | 11845 | OW0 | HOH | W | 16 | 21.430 | −10.953 | 30.374 | 1.00 | 37.74 | O |
| ATOM | 11848 | OW0 | HOH | W | 17 | 17.557 | 21.535 | 6.103 | 1.00 | 31.67 | O |
| ATOM | 11851 | OW0 | HOH | W | 18 | 25.257 | −18.527 | −8.265 | 1.00 | 43.54 | O |
| ATOM | 11854 | OW0 | HOH | W | 19 | 14.576 | 17.289 | −6.788 | 1.00 | 33.78 | O |
| ATOM | 11857 | OW0 | HOH | W | 20 | 17.021 | −32.347 | 7.412 | 1.00 | 37.49 | O |
| ATOM | 11860 | OW0 | HOH | W | 21 | 33.191 | −30.915 | 11.497 | 1.00 | 50.67 | O |
| ATOM | 11863 | OW0 | HOH | W | 22 | 15.544 | −6.948 | −27.858 | 1.00 | 35.60 | O |
| ATOM | 11866 | OW0 | HOH | W | 23 | 12.351 | −17.474 | −0.360 | 1.00 | 30.45 | O |
| ATOM | 11869 | OW0 | HOH | W | 24 | 26.518 | −33.704 | 9.194 | 1.00 | 49.06 | O |
| ATOM | 11872 | OW0 | HOH | W | 25 | 20.665 | 28.429 | −21.743 | 1.00 | 53.51 | O |
| ATOM | 11875 | OW0 | HOH | W | 26 | 31.381 | −26.477 | 5.569 | 1.00 | 37.88 | O |
| ATOM | 11878 | OW0 | HOH | W | 27 | 33.637 | 18.716 | 6.609 | 1.00 | 36.28 | O |
| ATOM | 11881 | OW0 | HOH | W | 28 | 22.871 | 14.801 | 9.718 | 1.00 | 37.51 | O |
| ATOM | 11884 | OW0 | HOH | W | 29 | −14.119 | −14.524 | −14.803 | 1.00 | 46.49 | O |
| ATOM | 11887 | OW0 | HOH | W | 30 | 8.824 | 9.832 | −35.731 | 1.00 | 42.29 | O |
| ATOM | 11890 | OW0 | HOH | W | 31 | 5.101 | −33.773 | −25.543 | 1.00 | 55.88 | O |
| ATOM | 11893 | OW0 | HOH | W | 32 | 19.643 | −14.418 | 22.546 | 1.00 | 35.00 | O |
| ATOM | 11896 | OW0 | HOH | W | 33 | 0.754 | −28.421 | −1.063 | 1.00 | 38.41 | O |
| ATOM | 11899 | OW0 | HOH | W | 34 | 13.475 | 15.714 | 17.634 | 1.00 | 50.42 | O |
| ATOM | 11902 | OW0 | HOH | W | 35 | 6.725 | −38.497 | −17.225 | 1.00 | 34.70 | O |
| ATOM | 11905 | OW0 | HOH | W | 36 | 4.092 | 6.194 | −15.543 | 1.00 | 39.02 | O |
| ATOM | 11908 | OW0 | HOH | W | 37 | 0.259 | 24.963 | −4.915 | 1.00 | 57.63 | O |
| ATOM | 11911 | OW0 | HOH | W | 38 | 12.803 | 11.299 | −30.578 | 1.00 | 52.69 | O |
| ATOM | 11914 | OW0 | HOH | W | 39 | 25.882 | 21.602 | 24.289 | 1.00 | 38.18 | O |
| ATOM | 11917 | OW0 | HOH | W | 40 | 21.029 | −16.430 | 7.776 | 1.00 | 34.44 | O |
| ATOM | 11920 | OW0 | HOH | W | 41 | −6.549 | 6.375 | −15.682 | 1.00 | 51.51 | O |
| ATOM | 11923 | OW0 | HOH | W | 42 | 9.116 | −18.630 | 7.864 | 1.00 | 34.52 | O |
| ATOM | 11926 | OW0 | HOH | W | 43 | −2.759 | 20.741 | −15.744 | 1.00 | 43.12 | O |
| ATOM | 11929 | OW0 | HOH | W | 44 | 14.353 | −2.405 | 24.505 | 1.00 | 44.82 | O |
| ATOM | 11932 | OW0 | HOH | W | 45 | 33.596 | 26.591 | −7.772 | 1.00 | 66.53 | O |
| ATOM | 11935 | OW0 | HOH | W | 46 | 33.431 | 19.788 | −6.332 | 1.00 | 66.40 | O |
| ATOM | 11938 | OW0 | HOH | W | 47 | 37.644 | −10.092 | 10.231 | 1.00 | 67.36 | O |
| ATOM | 11941 | OW0 | HOH | W | 48 | 7.796 | 20.112 | 4.606 | 1.00 | 37.15 | O |
| ATOM | 11944 | OW0 | HOH | W | 49 | 22.738 | −13.950 | 19.526 | 1.00 | 34.75 | O |
| ATOM | 11947 | OW0 | HOH | W | 50 | −2.423 | −18.253 | −15.471 | 1.00 | 37.73 | O |
| ATOM | 11950 | OW0 | HOH | W | 51 | 22.016 | 5.711 | 17.530 | 1.00 | 36.97 | O |
| ATOM | 11953 | OW0 | HOH | W | 52 | 0.414 | −36.445 | −13.675 | 1.00 | 43.87 | O |
| ATOM | 11956 | OW0 | HOH | W | 53 | 17.946 | 24.029 | −23.572 | 1.00 | 49.51 | O |
| ATOM | 11959 | OW0 | HOH | W | 55 | 3.778 | 13.503 | −31.968 | 1.00 | 36.84 | O |
| ATOM | 11962 | OW0 | HOH | W | 56 | 28.389 | −25.259 | 19.638 | 1.00 | 44.15 | O |
| ATOM | 11965 | OW0 | HOH | W | 57 | 8.968 | 2.110 | −15.421 | 1.00 | 31.73 | O |
| ATOM | 11968 | OW0 | HOH | W | 58 | 15.930 | −29.236 | 27.883 | 1.00 | 88.04 | O |
| ATOM | 11971 | OW0 | HOH | W | 59 | 38.368 | −5.173 | 22.025 | 1.00 | 66.68 | O |
| ATOM | 11974 | OW0 | HOH | W | 60 | 0.565 | 27.718 | −5.784 | 1.00 | 37.16 | O |
| ATOM | 11977 | OW0 | HOH | W | 61 | −14.145 | 3.221 | −17.540 | 1.00 | 51.10 | O |
| ATOM | 11980 | OW0 | HOH | W | 62 | −1.029 | 24.952 | −20.486 | 1.00 | 55.57 | O |
| ATOM | 11983 | OW0 | HOH | W | 63 | 25.538 | 31.344 | 9.272 | 1.00 | 37.51 | O |
| ATOM | 11986 | OW0 | HOH | W | 64 | 11.543 | −30.627 | 7.646 | 1.00 | 63.89 | O |
| ATOM | 11989 | OW0 | HOH | W | 65 | −3.285 | −28.488 | −16.505 | 1.00 | 47.17 | O |
| ATOM | 11992 | OW0 | HOH | W | 66 | 35.121 | −6.617 | 46.599 | 1.00 | 64.40 | O |
| ATOM | 11995 | OW0 | HOH | W | 67 | 2.700 | 23.786 | 1.743 | 1.00 | 41.55 | O |
| ATOM | 11998 | OW0 | HOH | W | 68 | 17.267 | −1.883 | 33.019 | 1.00 | 77.81 | O |
| ATOM | 12001 | OW0 | HOH | W | 69 | 28.570 | 5.784 | 14.280 | 1.00 | 62.68 | O |
| ATOM | 12004 | OW0 | HOH | W | 70 | −6.930 | −11.465 | −15.897 | 1.00 | 49.59 | O |
| ATOM | 12007 | OW0 | HOH | W | 71 | 23.778 | −1.987 | 17.551 | 1.00 | 43.21 | O |
| ATOM | 12010 | OW0 | HOH | W | 72 | −8.046 | 8.093 | −7.047 | 1.00 | 67.88 | O |
| ATOM | 12013 | OW0 | HOH | W | 73 | 12.439 | 27.161 | 14.625 | 1.00 | 53.32 | O |
| ATOM | 12016 | OW0 | HOH | W | 74 | 29.531 | −29.263 | 0.458 | 1.00 | 47.20 | O |
| ATOM | 12019 | OW0 | HOH | W | 75 | −12.691 | 4.754 | −30.109 | 1.00 | 60.79 | O |
| ATOM | 12022 | OW0 | HOH | W | 76 | 45.628 | 14.303 | 12.989 | 1.00 | 45.97 | O |
| ATOM | 12025 | OW0 | HOH | W | 77 | 8.533 | 25.938 | −23.895 | 1.00 | 60.13 | O |
| ATOM | 12028 | OW0 | HOH | W | 78 | 33.480 | −30.274 | 14.799 | 1.00 | 52.43 | O |
| ATOM | 12031 | OW0 | HOH | W | 79 | 13.424 | −38.072 | −13.361 | 1.00 | 58.64 | O |
| ATOM | 12034 | OW0 | HOH | W | 80 | −2.105 | 20.940 | −19.571 | 1.00 | 48.23 | O |
| ATOM | 12037 | OW0 | HOH | W | 81 | 33.669 | −10.905 | 42.251 | 1.00 | 60.73 | O |
| ATOM | 12040 | OW0 | HOH | W | 82 | −1.730 | 12.610 | −16.476 | 1.00 | 54.91 | O |
| ATOM | 12043 | OW0 | HOH | W | 83 | 8.302 | 14.887 | −3.529 | 1.00 | 42.05 | O |
| ATOM | 12046 | OW0 | HOH | W | 84 | 12.309 | 4.456 | −30.067 | 1.00 | 37.77 | O |
| ATOM | 12049 | OW0 | HOH | W | 85 | 0.258 | −0.895 | −11.065 | 1.00 | 50.59 | O |
| ATOM | 12052 | OW0 | HOH | W | 86 | 10.583 | −27.709 | 7.457 | 1.00 | 44.77 | O |
| ATOM | 12055 | OW0 | HOH | W | 87 | 15.029 | 11.505 | −13.957 | 1.00 | 47.49 | O |
| ATOM | 12058 | OW0 | HOH | W | 88 | −1.663 | 5.027 | −10.558 | 1.00 | 40.09 | O |
| ATOM | 12061 | OW0 | HOH | W | 89 | 14.576 | 6.271 | 33.384 | 1.00 | 73.40 | O |
| ATOM | 12064 | OW0 | HOH | W | 91 | 3.731 | −8.648 | −16.122 | 1.00 | 38.03 | O |
| ATOM | 12067 | OW0 | HOH | W | 92 | −2.084 | −17.659 | −3.768 | 1.00 | 67.74 | O |
| ATOM | 12070 | OW0 | HOH | W | 93 | 30.558 | 30.499 | 36.070 | 1.00 | 50.69 | O |
| ATOM | 12073 | OW0 | HOH | W | 95 | 5.176 | −33.114 | −21.314 | 1.00 | 41.67 | O |
| ATOM | 12076 | OW0 | HOH | W | 96 | 14.054 | −10.209 | 26.725 | 1.00 | 62.57 | O |
| ATOM | 12079 | OW0 | HOH | W | 97 | 34.005 | −20.627 | 14.186 | 1.00 | 54.51 | O |
| ATOM | 12082 | OW0 | HOH | W | 98 | 10.602 | −25.526 | 17.503 | 1.00 | 61.88 | O |
| ATOM | 12085 | OW0 | HOH | W | 99 | 13.731 | 38.986 | 21.926 | 1.00 | 60.08 | O |
| ATOM | 12088 | OW0 | HOH | W | 100 | 6.424 | 23.864 | 9.709 | 1.00 | 60.19 | O |
| ATOM | 12091 | OW0 | HOH | W | 101 | 36.228 | 1.373 | 41.492 | 1.00 | 66.08 | O |
| ATOM | 12094 | OW0 | HOH | W | 102 | 13.437 | 11.225 | −16.447 | 1.00 | 51.30 | O |
| ATOM | 12097 | OW0 | HOH | W | 103 | −1.923 | 12.954 | −18.725 | 1.00 | 49.86 | O |
| ATOM | 12100 | OW0 | HOH | W | 104 | 31.987 | −18.852 | 5.429 | 1.00 | 39.81 | O |
| ATOM | 12103 | OW0 | HOH | W | 105 | 24.933 | −25.093 | −10.983 | 1.00 | 45.17 | O |
| ATOM | 12106 | OW0 | HOH | W | 106 | 14.263 | 12.654 | 25.148 | 1.00 | 54.77 | O |
| ATOM | 12109 | OW0 | HOH | W | 107 | 17.903 | −34.761 | −20.461 | 1.00 | 66.90 | O |
| ATOM | 12112 | OW0 | HOH | W | 108 | 22.185 | 17.388 | −20.993 | 1.00 | 62.39 | O |
| ATOM | 12115 | OW0 | HOH | W | 109 | 12.088 | 39.585 | −15.367 | 1.00 | 58.49 | O |
| ATOM | 12118 | OW0 | HOH | W | 110 | 17.651 | −16.457 | 27.293 | 1.00 | 95.52 | O |
| ATOM | 12121 | OW0 | HOH | W | 111 | 42.960 | −0.638 | 34.927 | 1.00 | 53.79 | O |
| ATOM | 12124 | OW0 | HOH | W | 112 | 0.020 | −2.969 | −37.097 | 1.00 | 50.87 | O |
| ATOM | 12127 | OW0 | HOH | W | 113 | 31.273 | −15.609 | 28.075 | 1.00 | 46.77 | O |
| ATOM | 12130 | OW0 | HOH | W | 114 | 5.848 | −32.025 | 1.780 | 1.00 | 36.34 | O |
| ATOM | 12133 | OW0 | HOH | W | 115 | 25.264 | −22.691 | −12.173 | 1.00 | 54.23 | O |
| ATOM | 12136 | OW0 | HOH | W | 116 | 14.931 | −7.828 | 17.146 | 1.00 | 39.60 | O |
| ATOM | 12139 | OW0 | HOH | W | 117 | 14.450 | −15.069 | 0.942 | 1.00 | 47.27 | O |
| ATOM | 12142 | OW0 | HOH | W | 118 | 14.909 | 10.741 | 18.545 | 1.00 | 42.78 | O |
| ATOM | 12145 | OW0 | HOH | W | 119 | 4.879 | −32.660 | −23.697 | 1.00 | 45.49 | O |
| ATOM | 12148 | OW0 | HOH | W | 120 | 35.420 | −5.160 | 38.467 | 1.00 | 60.66 | O |
| ATOM | 12151 | OW0 | HOH | W | 121 | 19.085 | −38.737 | −15.382 | 1.00 | 82.42 | O |
| ATOM | 12154 | OW0 | HOH | W | 123 | 15.267 | 35.533 | 1.416 | 1.00 | 42.69 | O |
| ATOM | 12157 | OW0 | HOH | W | 124 | 17.066 | 31.782 | 5.456 | 1.00 | 38.99 | O |
| ATOM | 12160 | OW0 | HOH | W | 125 | 21.174 | 27.021 | −12.468 | 1.00 | 132.41 | O |
| ATOM | 12163 | OW0 | HOH | W | 126 | 5.064 | 13.701 | 20.548 | 1.00 | 65.72 | O |
| ATOM | 12166 | OW0 | HOH | W | 127 | −6.100 | −4.970 | −29.687 | 1.00 | 81.91 | O |
| ATOM | 12169 | OW0 | HOH | W | 128 | 14.862 | −33.589 | −8.189 | 1.00 | 77.42 | O |
| ATOM | 12172 | OW0 | HOH | W | 129 | 11.245 | −23.375 | 29.955 | 1.00 | 59.28 | O |
| ATOM | 12175 | OW0 | HOH | W | 130 | 7.347 | 1.217 | −35.209 | 1.00 | 37.93 | O |
| ATOM | 12178 | OW0 | HOH | W | 131 | −5.104 | −32.385 | −13.615 | 1.00 | 50.08 | O |
| ATOM | 12181 | OW0 | HOH | W | 132 | 13.393 | 25.682 | 5.192 | 1.00 | 36.05 | O |
| ATOM | 12184 | OW0 | HOH | W | 133 | 3.253 | −39.566 | −7.523 | 1.00 | 65.65 | O |
| ATOM | 12187 | OW0 | HOH | W | 134 | −0.168 | 11.960 | −24.648 | 1.00 | 35.36 | O |
| ATOM | 12190 | OW0 | HOH | W | 135 | 0.618 | 37.129 | −9.266 | 1.00 | 54.46 | O |
| ATOM | 12193 | OW0 | HOH | W | 136 | 24.019 | −33.829 | −8.776 | 1.00 | 65.33 | O |
| ATOM | 12196 | OW0 | HOH | W | 137 | 33.968 | 0.683 | 14.425 | 1.00 | 63.36 | O |
| ATOM | 12199 | OW0 | HOH | W | 138 | −13.520 | 4.414 | −24.874 | 1.00 | 50.94 | O |
| ATOM | 12202 | OW0 | HOH | W | 139 | 3.617 | −5.904 | −15.543 | 1.00 | 58.19 | O |
| ATOM | 12205 | OW0 | HOH | W | 140 | 5.668 | 35.253 | 2.617 | 1.00 | 46.14 | O |
| ATOM | 12208 | OW0 | HOH | W | 141 | 0.000 | −36.630 | 0.000 | 0.50 | 54.23 | O |
| ATOM | 12211 | OW0 | HOH | W | 142 | 8.593 | 46.925 | −7.114 | 1.00 | 48.24 | O |
| ATOM | 12214 | OW0 | HOH | W | 143 | 22.183 | 30.808 | −18.002 | 1.00 | 53.27 | O |
| ATOM | 12217 | OW0 | HOH | W | 144 | 9.621 | 31.955 | −22.810 | 1.00 | 62.85 | O |
| ATOM | 12220 | OW0 | HOH | W | 145 | −7.725 | −6.792 | −29.408 | 1.00 | 77.20 | O |
| ATOM | 12223 | OW0 | HOH | W | 146 | 25.843 | 31.204 | −11.116 | 1.00 | 89.49 | O |
| ATOM | 12226 | OW0 | HOH | W | 147 | 38.252 | −30.921 | 6.222 | 1.00 | 64.91 | O |
| ATOM | 12229 | OW0 | HOH | W | 148 | 32.698 | 17.606 | 2.019 | 1.00 | 59.14 | O |
| ATOM | 12232 | OW0 | HOH | W | 149 | 20.493 | −4.776 | 15.313 | 1.00 | 56.29 | O |
| ATOM | 12235 | OW0 | HOH | W | 150 | 15.944 | −32.061 | 10.364 | 1.00 | 109.14 | O |
| ATOM | 12238 | OW0 | HOH | W | 151 | 0.447 | −25.814 | −0.137 | 0.50 | 37.41 | O |
| ATOM | 12241 | OW0 | HOH | W | 152 | −14.381 | −1.582 | −14.435 | 1.00 | 46.71 | O |

TABLE 4
Coordinates of the complex of human PCNA of space group P3 with unit
dimensions: a = 82.89 Å, b = 82.89 Å, c = 70.86 Å.

| REMARK | 3 | REFINEMENT. | | |
|---|---|---|---|---|
| REMARK | 3 | PROGRAM : REFMAC 5.1.24 | | |
| REMARK | 3 | AUTHORS : MURSHUDOV, VAGIN, DODSON | | |
| REMARK | 3 | | | |
| REMARK | 3 | REFINEMENT TARGET: MAXIMUM LIKELIHOOD | | |
| REMARK | 3 | | | |
| REMARK | 3 | DATA USED IN REFINEMENT. | | |
| REMARK | 3 | RESOLUTION RANGE HIGH | (ANGSTROMS) | : 3.15 |
| REMARK | 3 | RESOLUTION RANGE LOW | (ANGSTROMS) | : 11.00 |
| REMARK | 3 | DATA CUTOFF | (SIGMA(F)) | : NONE |
| REMARK | 3 | COMPLETENESS FOR RANGE | (%) | : 98.10 |
| REMARK | 3 | NUMBER OF REFLECTIONS | | : 8605 |
| REMARK | 3 | | | |

| REMARK | 3 | FIT TO DATA USED IN REFINEMENT. | | |
|---|---|---|---|---|
| REMARK | 3 | CROSS-VALIDATION METHOD | | : THROUGHOUT |
| REMARK | 3 | FREE R VALUE TEST SET SELECTION | | : RANDOM |
| REMARK | 3 | R VALUE | (WORKING + TEST SET) | : 0.19695 |
| REMARK | 3 | R VALUE | (WORKING SET) | : 0.19314 |

| REMARK | 3 | FREE R VALUE | | : 0.27890 |
|---|---|---|---|---|
| REMARK | 3 | FREE R VALUE TEST SET SIZE | (%) | : 4.8 |
| REMARK | 3 | FREE R VALUE TEST SET COUNT | | : 430 |
| REMARK | 3 | | | |

| REMARK | 3 | FIT IN THE HIGHEST RESOLUTION BIN. | | |
|---|---|---|---|---|
| REMARK | 3 | TOTAL NUMBER OF BINS USED | | : 20 |
| REMARK | 3 | BIN RESOLUTION RANGE HIGH | | : 3.150 |
| REMARK | 3 | BIN RESOLUTION RANGE LOW | | : 3.225 |
| REMARK | 3 | REFLECTION IN BIN | (WORKING SET) | : 597 |
| REMARK | 3 | BIN R VALUE | (WORKING SET) | : 0.204 |

| | | | |
|---|---|---|---|
| REMARK | 3 | BIN FREE R VALUE SET COUNT | : 29 |
| REMARK | 3 | BIN FREE R VALUE | : 0.351 |
| REMARK | 3 | | |

| REMARK | 3 | NUMBER OF NON-HYDROGEN ATOMS USED IN REFINEMENT. | |
|---|---|---|---|
| REMARK | 3 | ALL ATOMS | : 3984 |
| REMARK | 3 | | |

| REMARK | 3 | B VALUES. | | |
|---|---|---|---|---|
| REMARK | 3 | FROM WILSON PLOT | (A**2) | : NULL |
| REMARK | 3 | MEAN B VALUE | (OVERALL, A**2) | : 50.798 |

| REMARK | 3 | OVERALL ANISOTROPIC B VALUE. | | |
|---|---|---|---|---|
| REMARK | 3 | B11 (A**2): −0.33 | | |
| REMARK | 3 | B22 (A**2): −0.33 | | |
| REMARK | 3 | B33 (A**2): 0.49 | | |
| REMARK | 3 | B12 (A**2): −0.16 | | |
| REMARK | 3 | B13 (A**2): 0.00 | | |
| REMARK | 3 | B23 (A**2): 0.00 | | |
| REMARK | 3 | | | |
| REMARK | 3 | ESTIMATED OVERALL COORDINATE ERROR. | | |
| REMARK | 3 | ESU BASED ON R VALUE | (A) | : NULL |
| REMARK | 3 | ESU BASED ON FREE R VALUE | (A) | : 0.612 |
| REMARK | 3 | ESU BASED ON MAXIMUM LIKELIHOOD | (A) | : 0.456 |
| REMARK | 3 | ESU FOR B VALUES BASED ON MAXIMUM LIKELIHOOD | (A**2) | : 26.459 |
| REMARK | 3 | | | |

| REMARK | 3 | CORRELATION COEFFICIENTS. | |
|---|---|---|---|
| REMARK | 3 | CORRELATION COEFFICIENT FO-FC | : 0.931 |
| REMARK | 3 | CORRELATION COEFFICIENT FO-FC FREE | : 0.833 |
| REMARK | 3 | | |

| REMARK | 3 | RMS DEVIATIONS FROM IDEAL VALUES | | COUNT | RMS | WEIGHT |
|---|---|---|---|---|---|---|
| REMARK | 3 | BOND LENGTHS REFINED ATOMS | (A) | : 3978 ; | 0.012 ; | 0.022 |

| | | | | | | |
|---|---|---|---|---|---|---|
| REMARK | 3 | BOND ANGLES REFINED ATOMS | (DEGREES) | : 5374 ; | 1.352 ; | 1.977 |
| REMARK | 3 | TORSION ANGLES, PERIOD 1 | (DEGREES) | : 508 ; | 8.350 ; | 5.000 |

| REMARK | 3 | CHIRAL-CENTER RESTRAINTS | (A**3) | : 636 ; | 0.088 ; | 0.200 |
|---|---|---|---|---|---|---|
| REMARK | 3 | GENERAL PLANES REFINED ATOMS | (A) | : 2904 ; | 0.011 ; | 0.020 |
| REMARK | 3 | NON-BONDED CONTACTS REFINED ATOMS | (A) | : 1590 ; | 0.255 ; | 0.300 |
| REMARK | 3 | H-BOND (X...Y) REFINED ATOMS | (A) | : 210 ; | 0.199 ; | 0.500 |
| REMARK | 3 | SYMMETRY VDW REFINED ATOMS | (A) | : 77 ; | 0.233 ; | 0.300 |
| REMARK | 3 | SYMMETRY H-BOND REFINED ATOMS | (A) | : 12 ; | 0.119 ; | 0.500 |
| REMARK | 3 | | | | | |
| REMARK | 3 | ISOTROPIC THERMAL FACTOR RESTRAINTS. | | COUNT | RMS | WEIGHT |
| REMARK | 3 | MAIN-CHAIN BOND REFINED ATOMS | (A**2) | : 2536 ; | 5.787 ; | 1.500 |
| REMARK | 3 | MAIN-CHAIN ANGLE REFINED ATOMS | (A**2) | : 4094 ; | 9.505 ; | 2.000 |
| REMARK | 3 | SIDE-CHAIN BOND REFINED ATOMS | (A**2) | : 1442 ; | 14.257 ; | 3.000 |
| REMARK | 3 | SIDE-CHAIN ANGLE REFINED ATOMS | (A**2) | : 1280 ; | 19.685 ; | 4.500 |
| REMARK | 3 | | | | | |

| REMARK | 3 | NCS RESTRAINTS STATISTICS | |
|---|---|---|---|
| REMARK | 3 | NUMBER OF DIFFERENT NCS GROUPS : 1 | |
| REMARK | 3 | | |
| REMARK | 3 | NCS GROUP NUMBER : 1 | |
| REMARK | 3 | CHAIN NAMES | : A B |
| REMARK | 3 | NUMBER OF COMPONENTS NCS GROUP | : 1 |

| | | | | | | | | |
|---|---|---|---|---|---|---|---|---|
| REMARK | 3 | COMPONENT | C | SSSEQI | TO | C | SSSEQI | CODE |
| REMARK | 3 | 1 | A | −9999 | | A | 99999 | 0 |
| REMARK | 3 | 1 | B | −9999 | | B | 99999 | 0 |

| REMARK | 3 | | GROUP | CHAIN | | COUNT | RMS | WEIGHT |
|---|---|---|---|---|---|---|---|---|
| REMARK | 3 | TIGHT POSITIONAL | 1 | A | (A) | : 1963 ; | 0.28 ; | 0.05 |
| REMARK | 3 | TIGHT THERMAL | 1 | A | (A**2) | : 1963 ; | 0.60 ; | 0.50 |
| REMARK | 3 | | | | | | | |
| REMARK | 3 | | | | | | | |

| REMARK | 3 | TLS DETAILS | |
|---|---|---|---|
| REMARK | 3 | NUMBER OF TLS GROUPS : NULL | |
| REMARK | 3 | | |
| REMARK | 3 | | |
| REMARK | 3 | BULK SOLVENT MODELLING. | |
| REMARK | 3 | METHOD USED: BABINET MODEL WITH MASK | |
| REMARK | 3 | PARAMETERS FOR MASK CALCULATION | |
| REMARK | 3 | VDW PROBE RADIUS | : 1.40 |
| REMARK | 3 | ION PROBE RADIUS | : 0.80 |
| REMARK | 3 | SHRINKAGE RADIUS | : 0.80 |
| REMARK | 3 | | |

| | | |
|---|---|---|
| REMARK | 3 | OTHER REFINEMENT REMARKS: NULL |
| REMARK | 3 | |

| | | | | | | | |
|---|---|---|---|---|---|---|---|
| CRYST1 | 82.892 | 82.892 | 70.859 | 90.00 | 90.00 | 120.00 | P 3 |

| | |
|---|---|
| SCALE1 | 0.012064 0.006965 0.000000 0.00000 |
| SCALE2 | 0.000000 0.013930 0.000000 0.00000 |
| SCALE3 | 0.000000 0.000000 0.014113 0.00000 |

| | | | | | | | | | | | |
|---|---|---|---|---|---|---|---|---|---|---|---|
| ATOM | 1 | N | MET | A | 1 | −0.829 | 12.134 | 0.858 | 1.00 | 55.18 | N |
| ATOM | 2 | CA | MET | A | 1 | 0.452 | 12.842 | 0.599 | 1.00 | 60.21 | C |
| ATOM | 3 | CB | MET | A | 1 | 0.341 | 14.299 | 1.066 | 1.00 | 62.15 | C |
| ATOM | 4 | CG | MET | A | 1 | 0.450 | 14.461 | 2.575 | 1.00 | 69.02 | C |
| ATOM | 5 | SD | MET | A | 1 | 1.028 | 16.083 | 3.081 | 1.00 | 98.72 | S |
| ATOM | 6 | CE | MET | A | 1 | −0.296 | 16.588 | 4.260 | 1.00 | 85.29 | C |
| ATOM | 7 | C | MET | A | 1 | 1.607 | 12.165 | 1.340 | 1.00 | 61.80 | C |
| ATOM | 8 | O | MET | A | 1 | 1.393 | 11.434 | 2.315 | 1.00 | 59.85 | O |
| ATOM | 9 | N | PHE | A | 2 | 2.836 | 12.491 | 0.927 | 1.00 | 58.05 | N |
| ATOM | 10 | CA | PHE | A | 2 | 4.054 | 11.954 | 1.554 | 1.00 | 53.35 | C |
| ATOM | 11 | CB | PHE | A | 2 | 5.002 | 11.385 | 0.481 | 1.00 | 55.06 | C |
| ATOM | 12 | CG | PHE | A | 2 | 6.461 | 11.331 | 0.901 | 1.00 | 64.53 | C |
| ATOM | 13 | CD1 | PHE | A | 2 | 6.838 | 10.693 | 2.097 | 1.00 | 53.09 | C |
| ATOM | 14 | CE1 | PHE | A | 2 | 8.177 | 10.639 | 2.494 | 1.00 | 38.48 | C |
| ATOM | 15 | CZ | PHE | A | 2 | 9.150 | 11.227 | 1.692 | 1.00 | 30.26 | C |
| ATOM | 16 | CE2 | PHE | A | 2 | 8.797 | 11.842 | 0.465 | 1.00 | 47.15 | C |
| ATOM | 17 | CD2 | PHE | A | 2 | 7.464 | 11.884 | 0.075 | 1.00 | 47.02 | C |
| ATOM | 18 | C | PHE | A | 2 | 4.768 | 13.016 | 2.385 | 1.00 | 42.88 | C |
| ATOM | 19 | O | PHE | A | 2 | 4.962 | 14.133 | 1.927 | 1.00 | 43.56 | O |
| ATOM | 20 | N | GLU | A | 3 | 5.153 | 12.696 | 3.611 | 1.00 | 36.54 | N |
| ATOM | 21 | CA | GLU | A | 3 | 5.773 | 13.724 | 4.442 | 1.00 | 46.48 | C |
| ATOM | 22 | CB | GLU | A | 3 | 4.720 | 14.598 | 5.150 | 1.00 | 43.01 | C |
| ATOM | 23 | CG | GLU | A | 3 | 5.316 | 15.641 | 6.113 | 1.00 | 59.00 | C |
| ATOM | 24 | CD | GLU | A | 3 | 4.343 | 16.773 | 6.428 | 1.00 | 78.87 | C |
| ATOM | 25 | OE1 | GLU | A | 3 | 3.448 | 17.040 | 5.598 | 1.00 | 93.42 | O |
| ATOM | 26 | OE2 | GLU | A | 3 | 4.480 | 17.403 | 7.503 | 1.00 | 65.71 | O |
| ATOM | 27 | C | GLU | A | 3 | 6.782 | 13.146 | 5.438 | 1.00 | 49.14 | C |
| ATOM | 28 | O | GLU | A | 3 | 6.407 | 12.405 | 6.356 | 1.00 | 53.62 | O |
| ATOM | 29 | N | ALA | A | 4 | 8.063 | 13.448 | 5.229 | 1.00 | 40.80 | N |
| ATOM | 30 | CA | ALA | A | 4 | 9.103 | 12.879 | 6.063 | 1.00 | 36.38 | C |
| ATOM | 31 | CB | ALA | A | 4 | 9.925 | 11.869 | 5.292 | 1.00 | 45.40 | C |
| ATOM | 32 | C | ALA | A | 4 | 9.965 | 14.001 | 6.564 | 1.00 | 34.23 | C |
| ATOM | 33 | O | ALA | A | 4 | 10.388 | 14.858 | 5.796 | 1.00 | 46.29 | O |
| ATOM | 34 | N | ARG | A | 5 | 10.153 | 14.038 | 7.874 | 1.00 | 31.16 | N |
| ATOM | 35 | CA | ARG | A | 5 | 10.893 | 15.116 | 8.528 | 1.00 | 37.81 | C |
| ATOM | 36 | CB | ARG | A | 5 | 10.012 | 15.827 | 9.564 | 1.00 | 42.32 | C |
| ATOM | 37 | CG | ARG | A | 5 | 10.527 | 17.209 | 10.012 | 1.00 | 45.66 | C |
| ATOM | 38 | CD | ARG | A | 5 | 9.608 | 17.952 | 10.971 | 1.00 | 36.51 | C |
| ATOM | 39 | NE | ARG | A | 5 | 10.030 | 17.733 | 12.350 | 1.00 | 33.99 | N |
| ATOM | 40 | CZ | ARG | A | 5 | 10.563 | 18.675 | 13.117 | 1.00 | 48.79 | C |
| ATOM | 41 | NH1 | ARG | A | 5 | 10.736 | 19.911 | 12.635 | 1.00 | 17.52 | N |
| ATOM | 42 | NH2 | ARG | A | 5 | 10.965 | 18.363 | 14.350 | 1.00 | 40.41 | N |
| ATOM | 43 | C | ARG | A | 5 | 12.142 | 14.588 | 9.228 | 1.00 | 38.40 | C |
| ATOM | 44 | O | ARG | A | 5 | 12.066 | 13.777 | 10.148 | 1.00 | 41.86 | O |
| ATOM | 45 | N | LEU | A | 6 | 13.303 | 15.027 | 8.775 | 1.00 | 35.66 | N |
| ATOM | 46 | CA | LEU | A | 6 | 14.533 | 14.461 | 9.282 | 1.00 | 36.59 | C |
| ATOM | 47 | CB | LEU | A | 6 | 15.376 | 13.942 | 8.130 | 1.00 | 45.48 | C |
| ATOM | 48 | CG | LEU | A | 6 | 16.666 | 13.236 | 8.544 | 1.00 | 46.95 | C |
| ATOM | 49 | CD1 | LEU | A | 6 | 16.255 | 11.969 | 9.282 | 1.00 | 45.57 | C |
| ATOM | 50 | CD2 | LEU | A | 6 | 17.486 | 12.929 | 7.284 | 1.00 | 58.21 | C |
| ATOM | 51 | C | LEU | A | 6 | 15.294 | 15.531 | 10.024 | 1.00 | 34.03 | C |
| ATOM | 52 | O | LEU | A | 6 | 15.601 | 16.589 | 9.470 | 1.00 | 31.48 | O |
| ATOM | 53 | N | VAL | A | 7 | 15.528 | 15.286 | 11.304 | 1.00 | 31.13 | N |
| ATOM | 54 | CA | VAL | A | 7 | 16.068 | 16.334 | 12.138 | 1.00 | 39.00 | C |
| ATOM | 55 | CB | VAL | A | 7 | 15.878 | 16.022 | 13.612 | 1.00 | 45.39 | C |
| ATOM | 56 | CG1 | VAL | A | 7 | 16.838 | 16.860 | 14.475 | 1.00 | 47.70 | C |
| ATOM | 57 | CG2 | VAL | A | 7 | 14.430 | 16.290 | 13.980 | 1.00 | 26.89 | C |
| ATOM | 58 | C | VAL | A | 7 | 17.540 | 16.512 | 11.824 | 1.00 | 44.62 | C |
| ATOM | 59 | O | VAL | A | 7 | 17.995 | 17.647 | 11.648 | 1.00 | 47.90 | O |
| ATOM | 60 | N | GLN | A | 8 | 18.277 | 15.398 | 11.768 | 1.00 | 50.76 | N |
| ATOM | 61 | CA | GLN | A | 8 | 19.648 | 15.381 | 11.223 | 1.00 | 51.52 | C |
| ATOM | 62 | CB | GLN | A | 8 | 20.393 | 14.095 | 11.622 | 1.00 | 38.24 | C |
| ATOM | 63 | CG | GLN | A | 8 | 20.957 | 14.072 | 13.037 | 1.00 | 66.08 | C |
| ATOM | 64 | CD | GLN | A | 8 | 22.020 | 12.987 | 13.252 | 1.00 | 84.46 | C |
| ATOM | 65 | OE1 | GLN | A | 8 | 23.194 | 13.209 | 12.960 | 1.00 | 79.03 | O |
| ATOM | 66 | NE2 | GLN | A | 8 | 21.621 | 11.845 | 13.820 | 1.00 | 76.48 | N |
| ATOM | 67 | C | GLN | A | 8 | 19.649 | 15.530 | 9.688 | 1.00 | 52.73 | C |
| ATOM | 68 | O | GLN | A | 8 | 19.557 | 14.536 | 8.958 | 1.00 | 62.56 | O |
| ATOM | 69 | N | GLY | A | 9 | 19.698 | 16.764 | 9.192 | 1.00 | 42.03 | N |
| ATOM | 70 | CA | GLY | A | 9 | 19.664 | 16.979 | 7.759 | 1.00 | 38.28 | C |
| ATOM | 71 | C | GLY | A | 9 | 20.960 | 16.486 | 7.128 | 1.00 | 40.23 | C |
| ATOM | 72 | O | GLY | A | 9 | 21.052 | 16.245 | 5.923 | 1.00 | 32.00 | O |
| ATOM | 73 | N | SER | A | 10 | 22.003 | 16.450 | 7.949 | 1.00 | 39.57 | N |
| ATOM | 74 | CA | SER | A | 10 | 23.340 | 16.252 | 7.433 | 1.00 | 31.88 | C |
| ATOM | 75 | CB | SER | A | 10 | 24.351 | 16.231 | 8.594 | 1.00 | 43.72 | C |
| ATOM | 76 | OG | SER | A | 10 | 23.886 | 15.403 | 9.653 | 1.00 | 57.14 | O |
| ATOM | 77 | C | SER | A | 10 | 23.329 | 14.913 | 6.733 | 1.00 | 29.26 | C |
| ATOM | 78 | O | SER | A | 10 | 24.190 | 14.640 | 5.897 | 1.00 | 40.82 | O |
| ATOM | 79 | N | ILE | A | 11 | 22.449 | 14.028 | 7.189 | 1.00 | 29.84 | N |
| ATOM | 80 | CA | ILE | A | 11 | 22.495 | 12.611 | 6.797 | 1.00 | 31.34 | C |
| ATOM | 81 | CB | ILE | A | 11 | 21.544 | 11.756 | 7.708 | 1.00 | 33.79 | C |
| ATOM | 82 | CG1 | ILE | A | 11 | 22.244 | 11.412 | 9.039 | 1.00 | 49.80 | C |
| ATOM | 83 | CD1 | ILE | A | 11 | 21.342 | 10.751 | 10.087 | 1.00 | 46.80 | C |
| ATOM | 84 | CG2 | ILE | A | 11 | 21.080 | 10.486 | 7.004 | 1.00 | 23.02 | C |
| ATOM | 85 | C | ILE | A | 11 | 22.089 | 12.527 | 5.334 | 1.00 | 19.47 | C |
| ATOM | 86 | O | ILE | A | 11 | 22.747 | 11.922 | 4.482 | 1.00 | 21.42 | O |
| ATOM | 87 | N | LEU | A | 12 | 21.055 | 13.278 | 5.034 | 1.00 | 19.60 | N |
| ATOM | 88 | CA | LEU | A | 12 | 20.532 | 13.284 | 3.691 | 1.00 | 28.38 | C |
| ATOM | 89 | CB | LEU | A | 12 | 19.198 | 14.007 | 3.694 | 1.00 | 29.49 | C |
| ATOM | 90 | CG | LEU | A | 12 | 18.340 | 13.762 | 2.462 | 1.00 | 33.66 | C |
| ATOM | 91 | CD1 | LEU | A | 12 | 17.939 | 12.288 | 2.400 | 1.00 | 50.84 | C |
| ATOM | 92 | CD2 | LEU | A | 12 | 17.117 | 14.672 | 2.515 | 1.00 | 30.85 | C |
| ATOM | 93 | C | LEU | A | 12 | 21.492 | 13.960 | 2.710 | 1.00 | 30.70 | C |
| ATOM | 94 | O | LEU | A | 12 | 21.532 | 13.644 | 1.533 | 1.00 | 37.53 | O |
| ATOM | 95 | N | LYS | A | 13 | 22.251 | 14.931 | 3.174 | 1.00 | 30.96 | N |
| ATOM | 96 | CA | LYS | A | 13 | 23.216 | 15.544 | 2.285 | 1.00 | 30.56 | C |
| ATOM | 97 | CB | LYS | A | 13 | 23.795 | 16.814 | 2.921 | 1.00 | 32.11 | C |
| ATOM | 98 | CG | LYS | A | 13 | 22.916 | 18.053 | 2.775 | 1.00 | 25.25 | C |
| ATOM | 99 | CD | LYS | A | 13 | 23.047 | 19.030 | 3.951 | 1.00 | 41.08 | C |
| ATOM | 100 | CE | LYS | A | 13 | 24.461 | 19.600 | 4.128 | 1.00 | 42.42 | C |
| ATOM | 101 | NZ | LYS | A | 13 | 24.684 | 20.642 | 3.113 | 1.00 | 22.31 | N |
| ATOM | 102 | C | LYS | A | 13 | 24.318 | 14.512 | 2.039 | 1.00 | 36.39 | C |
| ATOM | 103 | O | LYS | A | 13 | 24.912 | 14.490 | 0.964 | 1.00 | 43.18 | O |
| ATOM | 104 | N | LYS | A | 14 | 24.669 | 13.741 | 3.069 | 1.00 | 31.92 | N |
| ATOM | 105 | CA | LYS | A | 14 | 25.852 | 12.909 | 2.999 | 1.00 | 28.85 | C |
| ATOM | 106 | CB | LYS | A | 14 | 26.193 | 12.335 | 4.383 | 1.00 | 22.78 | C |
| ATOM | 107 | CG | LYS | A | 14 | 26.964 | 13.317 | 5.245 | 1.00 | 15.18 | C |
| ATOM | 108 | CD | LYS | A | 14 | 27.027 | 12.903 | 6.709 | 1.00 | 28.57 | C |
| ATOM | 109 | CE | LYS | A | 14 | 27.651 | 14.003 | 7.580 | 1.00 | 42.03 | C |
| ATOM | 110 | NZ | LYS | A | 14 | 29.134 | 13.771 | 7.814 | 1.00 | 49.29 | N |
| ATOM | 111 | C | LYS | A | 14 | 25.446 | 11.831 | 2.032 | 1.00 | 33.39 | C |
| ATOM | 112 | O | LYS | A | 14 | 26.299 | 11.312 | 1.291 | 1.00 | 37.96 | O |
| ATOM | 113 | N | VAL | A | 15 | 24.149 | 11.506 | 2.052 | 1.00 | 24.40 | N |
| ATOM | 114 | CA | VAL | A | 15 | 23.624 | 10.396 | 1.266 | 1.00 | 25.48 | C |
| ATOM | 115 | CB | VAL | A | 15 | 22.176 | 10.081 | 1.607 | 1.00 | 28.14 | C |
| ATOM | 116 | CG1 | VAL | A | 15 | 21.558 | 9.239 | 0.466 | 1.00 | 28.55 | C |
| ATOM | 117 | CG2 | VAL | A | 15 | 22.089 | 9.362 | 2.946 | 1.00 | 41.39 | C |
| ATOM | 118 | C | VAL | A | 15 | 23.670 | 10.686 | −0.223 | 1.00 | 19.60 | C |
| ATOM | 119 | O | VAL | A | 15 | 24.068 | 9.842 | −1.027 | 1.00 | 15.24 | O |
| ATOM | 120 | N | LEU | A | 16 | 23.316 | 11.918 | −0.558 | 1.00 | 22.33 | N |
| ATOM | 121 | CA | LEU | A | 16 | 23.215 | 12.363 | −1.942 | 1.00 | 30.44 | C |
| ATOM | 122 | CB | LEU | A | 16 | 22.308 | 13.599 | −2.072 | 1.00 | 37.58 | C |
| ATOM | 123 | CG | LEU | A | 16 | 21.198 | 13.630 | −3.141 | 1.00 | 43.08 | C |
| ATOM | 124 | CD1 | LEU | A | 16 | 20.176 | 12.524 | −2.901 | 1.00 | 41.55 | C |
| ATOM | 125 | CD2 | LEU | A | 16 | 20.503 | 14.974 | −3.209 | 1.00 | 35.60 | C |
| ATOM | 126 | C | LEU | A | 16 | 24.574 | 12.677 | −2.549 | 1.00 | 35.51 | C |
| ATOM | 127 | O | LEU | A | 16 | 24.758 | 12.511 | −3.751 | 1.00 | 51.12 | O |
| ATOM | 128 | N | GLU | A | 17 | 25.550 | 13.051 | −1.729 | 1.00 | 32.23 | N |
| ATOM | 129 | CA | GLU | A | 17 | 26.916 | 13.185 | −2.231 | 1.00 | 32.58 | C |
| ATOM | 130 | CB | GLU | A | 17 | 27.822 | 13.880 | −1.211 | 1.00 | 34.90 | C |
| ATOM | 131 | CG | GLU | A | 17 | 27.560 | 15.370 | −1.109 | 1.00 | 50.45 | C |
| ATOM | 132 | CD | GLU | A | 17 | 27.581 | 16.025 | −2.478 | 1.00 | 82.19 | C |
| ATOM | 133 | OE1 | GLU | A | 17 | 28.696 | 16.333 | −2.961 | 1.00 | 84.67 | O |
| ATOM | 134 | OE2 | GLU | A | 17 | 26.495 | 16.188 | −3.092 | 1.00 | 80.60 | O |
| ATOM | 135 | C | GLU | A | 17 | 27.495 | 11.838 | −2.624 | 1.00 | 33.73 | C |
| ATOM | 136 | O | GLU | A | 17 | 28.571 | 11.775 | −3.222 | 1.00 | 38.56 | O |
| ATOM | 137 | N | ALA | A | 18 | 26.779 | 10.776 | −2.255 | 1.00 | 36.47 | N |
| ATOM | 138 | CA | ALA | A | 18 | 27.282 | 9.408 | −2.319 | 1.00 | 39.55 | C |
| ATOM | 139 | CB | ALA | A | 18 | 26.791 | 8.610 | −1.120 | 1.00 | 36.18 | C |
| ATOM | 140 | C | ALA | A | 18 | 26.821 | 8.751 | −3.600 | 1.00 | 40.10 | C |
| ATOM | 141 | O | ALA | A | 18 | 27.412 | 7.776 | −4.082 | 1.00 | 50.12 | O |
| ATOM | 142 | N | LEU | A | 19 | 25.773 | 9.325 | −4.169 | 1.00 | 35.36 | N |
| ATOM | 143 | CA | LEU | A | 19 | 25.109 | 8.674 | −5.269 | 1.00 | 31.85 | C |
| ATOM | 144 | CB | LEU | A | 19 | 23.614 | 8.702 | −5.036 | 1.00 | 32.28 | C |
| ATOM | 145 | CG | LEU | A | 19 | 23.271 | 8.214 | −3.633 | 1.00 | 34.69 | C |
| ATOM | 146 | CD1 | LEU | A | 19 | 21.805 | 8.345 | −3.451 | 1.00 | 7.58 | C |
| ATOM | 147 | CD2 | LEU | A | 19 | 23.694 | 6.759 | −3.435 | 1.00 | 40.96 | C |
| ATOM | 148 | C | LEU | A | 19 | 25.462 | 9.420 | −6.524 | 1.00 | 34.26 | C |
| ATOM | 149 | O | LEU | A | 19 | 25.641 | 8.819 | −7.578 | 1.00 | 38.51 | O |
| ATOM | 150 | N | LYS | A | 20 | 25.643 | 10.729 | −6.390 | 1.00 | 37.81 | N |
| ATOM | 151 | CA | LYS | A | 20 | 25.654 | 11.618 | −7.556 | 1.00 | 37.28 | C |
| ATOM | 152 | CB | LYS | A | 20 | 25.615 | 13.097 | −7.140 | 1.00 | 25.69 | C |
| ATOM | 153 | CG | LYS | A | 20 | 26.816 | 13.538 | −6.327 | 1.00 | 39.38 | C |
| ATOM | 154 | CD | LYS | A | 20 | 27.309 | 14.923 | −6.744 | 1.00 | 34.23 | C |
| ATOM | 155 | CE | LYS | A | 20 | 28.698 | 15.180 | −6.173 | 1.00 | 40.30 | C |
| ATOM | 156 | NZ | LYS | A | 20 | 29.581 | 15.931 | −7.131 | 1.00 | 72.69 | N |
| ATOM | 157 | C | LYS | A | 20 | 26.849 | 11.338 | −8.466 | 1.00 | 40.12 | C |
| ATOM | 158 | O | LYS | A | 20 | 26.815 | 11.630 | −9.669 | 1.00 | 42.98 | O |
| ATOM | 159 | N | ASP | A | 21 | 27.899 | 10.765 | −7.880 | 1.00 | 44.04 | N |
| ATOM | 160 | CA | ASP | A | 21 | 29.125 | 10.487 | −8.613 | 1.00 | 45.44 | C |
| ATOM | 161 | CB | ASP | A | 21 | 30.334 | 10.552 | −7.678 | 1.00 | 45.63 | C |
| ATOM | 162 | CG | ASP | A | 21 | 30.714 | 11.994 | −7.320 | 1.00 | 64.51 | C |
| ATOM | 163 | OD1 | ASP | A | 21 | 30.505 | 12.897 | −8.172 | 1.00 | 68.75 | O |
| ATOM | 164 | OD2 | ASP | A | 21 | 31.182 | 12.340 | −6.205 | 1.00 | 70.81 | O |
| ATOM | 165 | C | ASP | A | 21 | 29.025 | 9.133 | −9.299 | 1.00 | 43.64 | C |
| ATOM | 166 | O | ASP | A | 21 | 29.684 | 8.879 | −10.302 | 1.00 | 46.86 | O |
| ATOM | 167 | N | LEU | A | 22 | 28.122 | 8.299 | −8.805 | 1.00 | 42.32 | N |
| ATOM | 168 | CA | LEU | A | 22 | 27.940 | 6.987 | −9.381 | 1.00 | 39.82 | C |
| ATOM | 169 | CB | LEU | A | 22 | 27.620 | 5.966 | −8.296 | 1.00 | 38.04 | C |
| ATOM | 170 | CG | LEU | A | 22 | 27.564 | 4.513 | −8.789 | 1.00 | 42.43 | C |
| ATOM | 171 | CD1 | LEU | A | 22 | 28.804 | 4.125 | −9.602 | 1.00 | 43.15 | C |
| ATOM | 172 | CD2 | LEU | A | 22 | 27.390 | 3.536 | −7.648 | 1.00 | 11.53 | C |
| ATOM | 173 | C | LEU | A | 22 | 26.824 | 6.985 | −10.396 | 1.00 | 46.93 | C |
| ATOM | 174 | O | LEU | A | 22 | 27.004 | 6.495 | −11.510 | 1.00 | 55.27 | O |
| ATOM | 175 | N | ILE | A | 23 | 25.655 | 7.474 | −9.989 | 1.00 | 50.98 | N |
| ATOM | 176 | CA | ILE | A | 23 | 24.464 | 7.486 | −10.842 | 1.00 | 49.44 | C |
| ATOM | 177 | CB | ILE | A | 23 | 23.446 | 6.453 | −10.326 | 1.00 | 52.93 | C |
| ATOM | 178 | CG1 | ILE | A | 23 | 23.184 | 6.627 | −8.826 | 1.00 | 46.37 | C |
| ATOM | 179 | CD1 | ILE | A | 23 | 22.207 | 5.575 | −8.269 | 1.00 | 50.76 | C |
| ATOM | 180 | CG2 | ILE | A | 23 | 23.967 | 5.042 | −10.576 | 1.00 | 64.52 | C |
| ATOM | 181 | C | ILE | A | 23 | 23.824 | 8.864 | −10.837 | 1.00 | 50.13 | C |
| ATOM | 182 | O | ILE | A | 23 | 23.996 | 9.606 | −9.873 | 1.00 | 57.30 | O |
| ATOM | 183 | N | ASN | A | 24 | 23.034 | 9.185 | −11.862 | 1.00 | 50.65 | N |
| ATOM | 184 | CA | ASN | A | 24 | 22.278 | 10.451 | −11.879 | 1.00 | 50.94 | C |
| ATOM | 185 | CB | ASN | A | 24 | 22.871 | 11.422 | −12.881 | 1.00 | 40.56 | C |
| ATOM | 186 | CG | ASN | A | 24 | 23.677 | 10.703 | −13.931 | 1.00 | 71.34 | C |
| ATOM | 187 | OD1 | ASN | A | 24 | 24.687 | 10.064 | −13.607 | 1.00 | 85.21 | O |
| ATOM | 188 | ND2 | ASN | A | 24 | 23.171 | 10.686 | −15.169 | 1.00 | 85.54 | N |
| ATOM | 189 | C | ASN | A | 24 | 20.801 | 10.334 | −12.198 | 1.00 | 50.31 | C |
| ATOM | 190 | O | ASN | A | 24 | 20.129 | 11.363 | −12.327 | 1.00 | 52.00 | O |
| ATOM | 191 | N | GLU | A | 25 | 20.320 | 9.122 | −12.473 | 1.00 | 57.37 | N |
| ATOM | 192 | CA | GLU | A | 25 | 18.919 | 8.966 | −12.857 | 1.00 | 58.47 | C |
| ATOM | 193 | CB | GLU | A | 25 | 18.787 | 8.613 | −14.343 | 1.00 | 63.08 | C |
| ATOM | 194 | CG | GLU | A | 25 | 19.385 | 9.627 | −15.310 | 1.00 | 71.79 | C |
| ATOM | 195 | CD | GLU | A | 25 | 18.360 | 10.631 | −15.796 | 1.00 | 88.46 | C |
| ATOM | 196 | OE1 | GLU | A | 25 | 17.646 | 10.327 | −16.779 | 1.00 | 101.68 | O |
| ATOM | 197 | OE2 | GLU | A | 25 | 18.274 | 11.725 | −15.199 | 1.00 | 92.88 | O |
| ATOM | 198 | C | GLU | A | 25 | 18.436 | 7.802 | −12.052 | 1.00 | 54.80 | C |
| ATOM | 199 | O | GLU | A | 25 | 18.843 | 6.675 | −12.315 | 1.00 | 64.68 | O |
| ATOM | 200 | N | ALA | A | 26 | 17.681 | 8.083 | −11.000 | 1.00 | 50.30 | N |
| ATOM | 201 | CA | ALA | A | 26 | 17.272 | 7.036 | −10.071 | 1.00 | 47.05 | C |
| ATOM | 202 | CB | ALA | A | 26 | 18.217 | 6.934 | −8.883 | 1.00 | 42.21 | C |
| ATOM | 203 | C | ALA | A | 26 | 15.868 | 7.277 | −9.588 | 1.00 | 47.21 | C |
| ATOM | 204 | O | ALA | A | 26 | 15.343 | 8.378 | −9.693 | 1.00 | 44.05 | O |
| ATOM | 205 | N | CYS | A | 27 | 15.256 | 6.218 | −9.074 | 1.00 | 55.11 | N |
| ATOM | 206 | CA | CYS | A | 27 | 13.859 | 6.268 | −8.662 | 1.00 | 58.39 | C |
| ATOM | 207 | CB | CYS | A | 27 | 13.042 | 5.177 | −9.385 | 1.00 | 56.11 | C |
| ATOM | 208 | SG | CYS | A | 27 | 11.294 | 5.063 | −8.923 | 1.00 | 87.11 | S |
| ATOM | 209 | C | CYS | A | 27 | 13.820 | 6.069 | −7.155 | 1.00 | 47.07 | C |
| ATOM | 210 | O | CYS | A | 27 | 14.480 | 5.173 | −6.636 | 1.00 | 44.54 | O |
| ATOM | 211 | N | TRP | A | 28 | 13.050 | 6.916 | −6.477 | 1.00 | 46.76 | N |
| ATOM | 212 | CA | TRP | A | 28 | 12.821 | 6.812 | −5.036 | 1.00 | 53.27 | C |
| ATOM | 213 | CB | TRP | A | 28 | 12.810 | 8.208 | −4.410 | 1.00 | 56.76 | C |
| ATOM | 214 | CG | TRP | A | 28 | 14.123 | 8.924 | −4.532 | 1.00 | 59.67 | C |
| ATOM | 215 | CD1 | TRP | A | 28 | 14.750 | 9.338 | −5.689 | 1.00 | 57.41 | C |
| ATOM | 216 | NE1 | TRP | A | 28 | 15.947 | 9.943 | −5.382 | 1.00 | 50.95 | N |
| ATOM | 217 | CE2 | TRP | A | 28 | 16.114 | 9.942 | −4.018 | 1.00 | 52.34 | C |
| ATOM | 218 | CD2 | TRP | A | 28 | 14.996 | 9.281 | −3.455 | 1.00 | 52.08 | C |
| ATOM | 219 | CE3 | TRP | A | 28 | 14.942 | 9.124 | −2.066 | 1.00 | 50.37 | C |
| ATOM | 220 | CZ3 | TRP | A | 28 | 15.968 | 9.647 | −1.293 | 1.00 | 59.34 | C |
| ATOM | 221 | CH2 | TRP | A | 28 | 17.072 | 10.274 | −1.882 | 1.00 | 53.70 | C |
| ATOM | 222 | CZ2 | TRP | A | 28 | 17.175 | 10.417 | −3.236 | 1.00 | 34.53 | C |
| ATOM | 223 | C | TRP | A | 28 | 11.528 | 6.072 | −4.679 | 1.00 | 53.17 | C |
| ATOM | 224 | O | TRP | A | 28 | 10.435 | 6.604 | −4.823 | 1.00 | 50.35 | O |
| ATOM | 225 | N | ASP | A | 29 | 11.654 | 4.840 | −4.204 | 1.00 | 50.68 | N |
| ATOM | 226 | CA | ASP | A | 29 | 10.485 | 4.066 | −3.841 | 1.00 | 52.44 | C |
| ATOM | 227 | CB | ASP | A | 29 | 10.804 | 2.585 | −3.918 | 1.00 | 59.06 | C |
| ATOM | 228 | CG | ASP | A | 29 | 11.135 | 2.144 | −5.314 | 1.00 | 68.92 | C |
| ATOM | 229 | OD1 | ASP | A | 29 | 10.942 | 2.948 | −6.253 | 1.00 | 55.72 | O |
| ATOM | 230 | OD2 | ASP | A | 29 | 11.590 | 1.006 | −5.562 | 1.00 | 87.41 | O |
| ATOM | 231 | C | ASP | A | 29 | 10.065 | 4.414 | −2.431 | 1.00 | 56.84 | C |
| ATOM | 232 | O | ASP | A | 29 | 10.745 | 4.085 | −1.464 | 1.00 | 58.98 | O |
| ATOM | 233 | N | ILE | A | 30 | 8.944 | 5.110 | −2.321 | 1.00 | 61.91 | N |
| ATOM | 234 | CA | ILE | A | 30 | 8.467 | 5.577 | −1.026 | 1.00 | 62.05 | C |
| ATOM | 235 | CB | ILE | A | 30 | 8.000 | 7.038 | −1.109 | 1.00 | 60.21 | C |
| ATOM | 236 | CG1 | ILE | A | 30 | 8.982 | 7.876 | −1.936 | 1.00 | 57.78 | C |
| ATOM | 237 | CD1 | ILE | A | 30 | 9.578 | 9.072 | −1.188 | 1.00 | 73.12 | C |
| ATOM | 238 | CG2 | ILE | A | 30 | 7.829 | 7.595 | 0.287 | 1.00 | 66.51 | C |
| ATOM | 239 | C | ILE | A | 30 | 7.280 | 4.737 | −0.620 | 1.00 | 62.44 | C |
| ATOM | 240 | O | ILE | A | 30 | 6.316 | 4.611 | −1.384 | 1.00 | 65.71 | O |
| ATOM | 241 | N | SER | A | 31 | 7.339 | 4.199 | 0.594 | 1.00 | 53.95 | N |
| ATOM | 242 | CA | SER | A | 31 | 6.206 | 3.474 | 1.156 | 1.00 | 59.35 | C |
| ATOM | 243 | CB | SER | A | 31 | 6.312 | 1.982 | 0.834 | 1.00 | 55.33 | C |
| ATOM | 244 | OG | SER | A | 31 | 7.383 | 1.395 | 1.559 | 1.00 | 57.26 | O |
| ATOM | 245 | C | SER | A | 31 | 6.177 | 3.664 | 2.667 | 1.00 | 67.23 | C |
| ATOM | 246 | O | SER | A | 31 | 6.989 | 4.409 | 3.226 | 1.00 | 74.25 | O |
| ATOM | 247 | N | SER | A | 32 | 5.271 | 2.957 | 3.331 | 1.00 | 67.45 | N |
| ATOM | 248 | CA | SER | A | 32 | 5.070 | 3.154 | 4.754 | 1.00 | 66.63 | C |
| ATOM | 249 | CB | SER | A | 32 | 3.790 | 2.476 | 5.206 | 1.00 | 66.64 | C |
| ATOM | 250 | OG | SER | A | 32 | 2.689 | 3.317 | 4.911 | 1.00 | 77.68 | O |
| ATOM | 251 | C | SER | A | 32 | 6.240 | 2.649 | 5.573 | 1.00 | 69.44 | C |
| ATOM | 252 | O | SER | A | 32 | 6.428 | 3.085 | 6.710 | 1.00 | 71.56 | O |
| ATOM | 253 | N | SER | A | 33 | 7.022 | 1.736 | 5.001 | 1.00 | 68.38 | N |
| ATOM | 254 | CA | SER | A | 33 | 8.174 | 1.174 | 5.707 | 1.00 | 70.07 | C |
| ATOM | 255 | CB | SER | A | 33 | 8.659 | −0.081 | 4.986 | 1.00 | 74.53 | C |
| ATOM | 256 | OG | SER | A | 33 | 9.586 | 0.254 | 3.952 | 1.00 | 80.65 | O |
| ATOM | 257 | C | SER | A | 33 | 9.315 | 2.190 | 5.755 | 1.00 | 66.88 | C |
| ATOM | 258 | O | SER | A | 33 | 10.154 | 2.194 | 6.666 | 1.00 | 53.78 | O |
| ATOM | 259 | N | GLY | A | 34 | 9.378 | 3.012 | 4.716 | 1.00 | 65.96 | N |
| ATOM | 260 | CA | GLY | A | 34 | 10.517 | 3.895 | 4.558 | 1.00 | 64.44 | C |
| ATOM | 261 | C | GLY | A | 34 | 10.834 | 4.257 | 3.123 | 1.00 | 57.52 | C |
| ATOM | 262 | O | GLY | A | 34 | 10.005 | 4.117 | 2.220 | 1.00 | 53.76 | O |
| ATOM | 263 | N | VAL | A | 35 | 12.031 | 4.804 | 2.946 | 1.00 | 53.89 | N |
| ATOM | 264 | CA | VAL | A | 35 | 12.539 | 5.144 | 1.628 | 1.00 | 58.45 | C |
| ATOM | 265 | CB | VAL | A | 35 | 13.335 | 6.440 | 1.673 | 1.00 | 60.62 | C |
| ATOM | 266 | CG1 | VAL | A | 35 | 13.112 | 7.223 | 0.372 | 1.00 | 54.15 | C |
| ATOM | 267 | CG2 | VAL | A | 35 | 12.975 | 7.229 | 2.936 | 1.00 | 58.68 | C |
| ATOM | 268 | C | VAL | A | 35 | 13.454 | 4.059 | 1.074 | 1.00 | 58.66 | C |
| ATOM | 269 | O | VAL | A | 35 | 14.314 | 3.513 | 1.779 | 1.00 | 52.48 | O |
| ATOM | 270 | N | ASN | A | 36 | 13.279 | 3.758 | −0.205 | 1.00 | 54.00 | N |
| ATOM | 271 | CA | ASN | A | 36 | 14.163 | 2.798 | −0.849 | 1.00 | 59.44 | C |
| ATOM | 272 | CB | ASN | A | 36 | 13.469 | 1.443 | −1.009 | 1.00 | 50.61 | C |
| ATOM | 273 | CG | ASN | A | 36 | 12.989 | 0.904 | 0.318 | 1.00 | 53.65 | C |
| ATOM | 274 | OD1 | ASN | A | 36 | 13.757 | 0.855 | 1.287 | 1.00 | 83.71 | O |
| ATOM | 275 | ND2 | ASN | A | 36 | 11.692 | 0.635 | 0.425 | 1.00 | 61.33 | N |
| ATOM | 276 | C | ASN | A | 36 | 14.678 | 3.327 | −2.177 | 1.00 | 63.45 | C |
| ATOM | 277 | O | ASN | A | 36 | 13.978 | 4.091 | −2.872 | 1.00 | 58.99 | O |
| ATOM | 278 | N | LEU | A | 37 | 15.925 | 2.980 | −2.490 | 1.00 | 55.19 | N |
| ATOM | 279 | CA | LEU | A | 37 | 16.424 | 3.277 | −3.820 | 1.00 | 50.45 | C |
| ATOM | 280 | CB | LEU | A | 37 | 17.179 | 4.600 | −3.818 | 1.00 | 47.29 | C |
| ATOM | 281 | CG | LEU | A | 37 | 17.564 | 5.078 | −5.221 | 1.00 | 47.49 | C |
| ATOM | 282 | CD1 | LEU | A | 37 | 16.862 | 6.388 | −5.539 | 1.00 | 60.26 | C |
| ATOM | 283 | CD2 | LEU | A | 37 | 19.083 | 5.232 | −5.301 | 1.00 | 57.58 | C |
| ATOM | 284 | C | LEU | A | 37 | 17.277 | 2.143 | −4.354 | 1.00 | 54.03 | C |
| ATOM | 285 | O | LEU | A | 37 | 18.083 | 1.565 | −3.623 | 1.00 | 59.00 | O |
| ATOM | 286 | N | GLN | A | 38 | 17.034 | 1.775 | −5.608 | 1.00 | 52.46 | N |
| ATOM | 287 | CA | GLN | A | 38 | 17.805 | 0.731 | −6.283 | 1.00 | 48.64 | C |
| ATOM | 288 | CB | GLN | A | 38 | 17.035 | −0.604 | −6.274 | 1.00 | 41.61 | C |
| ATOM | 289 | CG | GLN | A | 38 | 17.100 | −1.352 | −4.932 | 1.00 | 58.61 | C |
| ATOM | 290 | CD | GLN | A | 38 | 17.251 | −2.866 | −5.085 | 1.00 | 81.06 | C |
| ATOM | 291 | OE1 | GLN | A | 38 | 17.342 | −3.373 | −6.216 | 1.00 | 76.07 | O |
| ATOM | 292 | NE2 | GLN | A | 38 | 17.316 | −3.583 | −3.949 | 1.00 | 61.09 | N |
| ATOM | 293 | C | GLN | A | 38 | 18.085 | 1.184 | −7.718 | 1.00 | 46.88 | C |
| ATOM | 294 | O | GLN | A | 38 | 17.162 | 1.309 | −8.529 | 1.00 | 51.33 | O |
| ATOM | 295 | N | SER | A | 39 | 19.348 | 1.475 | −8.011 | 1.00 | 37.04 | N |
| ATOM | 296 | CA | SER | A | 39 | 19.750 | 1.768 | −9.383 | 1.00 | 38.44 | C |
| ATOM | 297 | CB | SER | A | 39 | 19.614 | 3.263 | −9.677 | 1.00 | 37.34 | C |
| ATOM | 298 | OG | SER | A | 39 | 19.054 | 3.486 | −10.962 | 1.00 | 34.98 | O |
| ATOM | 299 | C | SER | A | 39 | 21.150 | 1.240 | −9.745 | 1.00 | 36.56 | C |
| ATOM | 300 | O | SER | A | 39 | 21.939 | 0.909 | −8.871 | 1.00 | 45.89 | O |
| ATOM | 301 | N | MET | A | 40 | 21.425 | 1.044 | −11.029 | 1.00 | 31.61 | N |
| ATOM | 302 | CA | MET | A | 40 | 22.760 | 0.653 | −11.469 | 1.00 | 25.45 | C |
| ATOM | 303 | CB | MET | A | 40 | 22.670 | −0.526 | −12.438 | 1.00 | 26.15 | C |
| ATOM | 304 | CG | MET | A | 40 | 21.680 | −1.605 | −11.991 | 1.00 | 52.02 | C |
| ATOM | 305 | SD | MET | A | 40 | 21.081 | −2.598 | −13.366 | 1.00 | 55.62 | S |
| ATOM | 306 | CE | MET | A | 40 | 22.471 | −3.648 | −13.562 | 1.00 | 42.64 | C |
| ATOM | 307 | C | MET | A | 40 | 23.393 | 1.813 | −12.207 | 1.00 | 22.87 | C |
| ATOM | 308 | O | MET | A | 40 | 22.712 | 2.775 | −12.593 | 1.00 | 36.02 | O |
| ATOM | 309 | N | ASP | A | 41 | 24.658 | 1.653 | −12.558 | 1.00 | 5.12 | N |
| ATOM | 310 | CA | ASP | A | 41 | 25.301 | 2.681 | −13.342 | 1.00 | 21.69 | C |
| ATOM | 311 | CB | ASP | A | 41 | 26.799 | 2.692 | −13.078 | 1.00 | 18.60 | C |
| ATOM | 312 | CG | ASP | A | 41 | 27.486 | 1.473 | −13.652 | 1.00 | 33.35 | C |
| ATOM | 313 | OD1 | ASP | A | 41 | 27.107 | 0.336 | −13.273 | 1.00 | 49.11 | O |
| ATOM | 314 | OD2 | ASP | A | 41 | 28.410 | 1.565 | −14.480 | 1.00 | 21.72 | O |
| ATOM | 315 | C | ASP | A | 41 | 25.017 | 2.532 | −14.842 | 1.00 | 27.89 | C |
| ATOM | 316 | O | ASP | A | 41 | 24.243 | 1.672 | −15.248 | 1.00 | 30.72 | O |
| ATOM | 317 | N | SER | A | 42 | 25.652 | 3.393 | −15.646 | 1.00 | 29.41 | N |
| ATOM | 318 | CA | SER | A | 42 | 25.622 | 3.298 | −17.100 | 1.00 | 29.59 | C |
| ATOM | 319 | CB | SER | A | 42 | 26.552 | 4.358 | −17.712 | 1.00 | 25.13 | C |
| ATOM | 320 | OG | SER | A | 42 | 25.861 | 5.584 | −17.814 | 1.00 | 33.34 | O |
| ATOM | 321 | C | SER | A | 42 | 26.005 | 1.900 | −17.603 | 1.00 | 35.06 | C |
| ATOM | 322 | O | SER | A | 42 | 25.269 | 1.291 | −18.381 | 1.00 | 35.81 | O |
| ATOM | 323 | N | SER | A | 43 | 27.190 | 1.428 | −17.206 | 1.00 | 42.18 | N |
| ATOM | 324 | CA | SER | A | 43 | 27.762 | 0.201 | −17.760 | 1.00 | 38.72 | C |
| ATOM | 325 | CB | SER | A | 43 | 29.289 | 0.216 | −17.669 | 1.00 | 42.66 | C |
| ATOM | 326 | OG | SER | A | 43 | 29.704 | 0.239 | −16.296 | 1.00 | 46.21 | O |
| ATOM | 327 | C | SER | A | 43 | 27.240 | −1.012 | −17.027 | 1.00 | 32.73 | C |
| ATOM | 328 | O | SER | A | 43 | 27.657 | −2.120 | −17.303 | 1.00 | 24.54 | O |
| ATOM | 329 | N | HIS | A | 44 | 26.292 | −0.789 | −16.123 | 1.00 | 43.33 | N |
| ATOM | 330 | CA | HIS | A | 44 | 25.640 | −1.864 | −15.373 | 1.00 | 43.18 | C |
| ATOM | 331 | CB | HIS | A | 44 | 24.759 | −2.725 | −16.287 | 1.00 | 43.88 | C |
| ATOM | 332 | CG | HIS | A | 44 | 23.444 | −2.100 | −16.642 | 1.00 | 54.21 | C |
| ATOM | 333 | ND1 | HIS | A | 44 | 22.372 | −2.833 | −17.111 | 1.00 | 24.39 | N |
| ATOM | 334 | CE1 | HIS | A | 44 | 21.370 | −2.020 | −17.392 | 1.00 | 47.85 | C |
| ATOM | 335 | NE2 | HIS | A | 44 | 21.764 | −0.780 | −17.160 | 1.00 | 58.88 | N |
| ATOM | 336 | CD2 | HIS | A | 44 | 23.066 | −0.800 | −16.716 | 1.00 | 67.80 | C |
| ATOM | 337 | C | HIS | A | 44 | 26.637 | −2.780 | −14.666 | 1.00 | 43.16 | C |
| ATOM | 338 | O | HIS | A | 44 | 26.422 | −3.998 | −14.614 | 1.00 | 44.15 | O |
| ATOM | 339 | N | VAL | A | 45 | 27.713 | −2.224 | −14.111 | 1.00 | 36.49 | N |
| ATOM | 340 | CA | VAL | A | 45 | 28.618 | −3.066 | −13.345 | 1.00 | 33.90 | C |
| ATOM | 341 | CB | VAL | A | 45 | 30.077 | −2.700 | −13.568 | 1.00 | 32.83 | C |
| ATOM | 342 | CG1 | VAL | A | 45 | 30.934 | −3.919 | −13.311 | 1.00 | 15.86 | C |
| ATOM | 343 | CG2 | VAL | A | 45 | 30.274 | −2.175 | −14.974 | 1.00 | 47.72 | C |
| ATOM | 344 | C | VAL | A | 45 | 28.357 | −2.984 | −11.859 | 1.00 | 43.06 | C |
| ATOM | 345 | O | VAL | A | 45 | 28.671 | −3.937 | −11.143 | 1.00 | 43.33 | O |
| ATOM | 346 | N | SER | A | 46 | 27.885 | −1.825 | −11.386 | 1.00 | 41.61 | N |
| ATOM | 347 | CA | SER | A | 46 | 27.640 | −1.633 | −9.964 | 1.00 | 37.09 | C |
| ATOM | 348 | CB | SER | A | 46 | 28.485 | −0.498 | −9.422 | 1.00 | 39.51 | C |
| ATOM | 349 | OG | SER | A | 46 | 27.957 | 0.733 | −9.875 | 1.00 | 44.45 | O |
| ATOM | 350 | C | SER | A | 46 | 26.183 | −1.320 | −9.723 | 1.00 | 46.31 | C |
| ATOM | 351 | O | SER | A | 46 | 25.468 | −0.893 | −10.627 | 1.00 | 49.82 | O |
| ATOM | 352 | N | LEU | A | 47 | 25.755 | −1.542 | −8.483 | 1.00 | 57.09 | N |
| ATOM | 353 | CA | LEU | A | 47 | 24.385 | −1.282 | −8.043 | 1.00 | 50.88 | C |
| ATOM | 354 | CB | LEU | A | 47 | 23.629 | −2.591 | −7.913 | 1.00 | 41.58 | C |
| ATOM | 355 | CG | LEU | A | 47 | 22.130 | −2.353 | −7.836 | 1.00 | 52.03 | C |
| ATOM | 356 | CD1 | LEU | A | 47 | 21.404 | −3.323 | −8.736 | 1.00 | 54.73 | C |
| ATOM | 357 | CD2 | LEU | A | 47 | 21.690 | −2.534 | −6.406 | 1.00 | 58.70 | C |
| ATOM | 358 | C | LEU | A | 47 | 24.375 | −0.566 | −6.696 | 1.00 | 54.13 | C |
| ATOM | 359 | O | LEU | A | 47 | 25.249 | −0.793 | −5.840 | 1.00 | 59.65 | O |
| ATOM | 360 | N | VAL | A | 48 | 23.399 | 0.322 | −6.521 | 1.00 | 50.56 | N |
| ATOM | 361 | CA | VAL | A | 48 | 23.209 | 1.002 | −5.245 | 1.00 | 40.93 | C |
| ATOM | 362 | CB | VAL | A | 48 | 23.101 | 2.526 | −5.399 | 1.00 | 32.99 | C |
| ATOM | 363 | CG1 | VAL | A | 48 | 23.628 | 3.199 | −4.155 | 1.00 | 49.25 | C |
| ATOM | 364 | CG2 | VAL | A | 48 | 23.881 | 3.021 | −6.594 | 1.00 | 54.47 | C |
| ATOM | 365 | C | VAL | A | 48 | 21.919 | 0.488 | −4.639 | 1.00 | 46.10 | C |
| ATOM | 366 | O | VAL | A | 48 | 20.929 | 0.281 | −5.344 | 1.00 | 51.05 | O |
| ATOM | 367 | N | GLN | A | 49 | 21.926 | 0.284 | −3.327 | 1.00 | 48.48 | N |
| ATOM | 368 | CA | GLN | A | 49 | 20.708 | −0.101 | −2.631 | 1.00 | 53.13 | C |
| ATOM | 369 | CB | GLN | A | 49 | 20.775 | −1.591 | −2.275 | 1.00 | 57.63 | C |
| ATOM | 370 | CG | GLN | A | 49 | 19.436 | −2.269 | −1.965 | 1.00 | 54.46 | C |
| ATOM | 371 | CD | GLN | A | 49 | 19.063 | −2.143 | −0.479 | 1.00 | 63.33 | C |
| ATOM | 372 | OE1 | GLN | A | 49 | 19.574 | −2.860 | 0.384 | 1.00 | 63.27 | O |
| ATOM | 373 | NE2 | GLN | A | 49 | 18.164 | −1.226 | −0.185 | 1.00 | 57.48 | N |
| ATOM | 374 | C | GLN | A | 49 | 20.588 | 0.754 | −1.377 | 1.00 | 53.82 | C |
| ATOM | 375 | O | GLN | A | 49 | 21.221 | 0.461 | −0.356 | 1.00 | 62.20 | O |
| ATOM | 376 | N | LEU | A | 50 | 19.829 | 1.844 | −1.478 | 1.00 | 53.66 | N |
| ATOM | 377 | CA | LEU | A | 50 | 19.597 | 2.729 | −0.331 | 1.00 | 54.81 | C |
| ATOM | 378 | CB | LEU | A | 50 | 19.677 | 4.208 | −0.728 | 1.00 | 54.68 | C |
| ATOM | 379 | CG | LEU | A | 50 | 19.149 | 5.156 | 0.357 | 1.00 | 34.54 | C |
| ATOM | 380 | CD1 | LEU | A | 50 | 20.246 | 5.343 | 1.384 | 1.00 | 30.29 | C |
| ATOM | 381 | CD2 | LEU | A | 50 | 18.707 | 6.493 | −0.260 | 1.00 | 28.81 | C |
| ATOM | 382 | C | LEU | A | 50 | 18.274 | 2.455 | 0.390 | 1.00 | 52.15 | C |
| ATOM | 383 | O | LEU | A | 50 | 17.214 | 2.339 | −0.261 | 1.00 | 39.43 | O |
| ATOM | 384 | N | THR | A | 51 | 18.384 | 2.333 | 1.722 | 1.00 | 42.60 | N |
| ATOM | 385 | CA | THR | A | 51 | 17.267 | 2.072 | 2.633 | 1.00 | 42.27 | C |
| ATOM | 386 | CB | THR | A | 51 | 17.275 | 0.621 | 3.115 | 1.00 | 38.14 | C |
| ATOM | 387 | OG1 | THR | A | 51 | 17.050 | −0.259 | 2.009 | 1.00 | 46.58 | O |
| ATOM | 388 | CG2 | THR | A | 51 | 16.056 | 0.358 | 3.963 | 1.00 | 56.25 | C |
| ATOM | 389 | C | THR | A | 51 | 17.256 | 2.990 | 3.860 | 1.00 | 43.37 | C |
| ATOM | 390 | O | THR | A | 51 | 18.122 | 2.883 | 4.742 | 1.00 | 40.07 | O |
| ATOM | 391 | N | LEU | A | 52 | 16.220 | 3.832 | 3.937 | 1.00 | 45.85 | N |
| ATOM | 392 | CA | LEU | A | 52 | 15.960 | 4.692 | 5.093 | 1.00 | 43.98 | C |
| ATOM | 393 | CB | LEU | A | 52 | 15.904 | 6.168 | 4.674 | 1.00 | 44.37 | C |
| ATOM | 394 | CG | LEU | A | 52 | 17.205 | 6.758 | 4.108 | 1.00 | 47.38 | C |
| ATOM | 395 | CD1 | LEU | A | 52 | 17.020 | 8.126 | 3.536 | 1.00 | 7.70 | C |
| ATOM | 396 | CD2 | LEU | A | 52 | 18.336 | 6.756 | 5.128 | 1.00 | 50.98 | C |
| ATOM | 397 | C | LEU | A | 52 | 14.637 | 4.290 | 5.730 | 1.00 | 48.48 | C |
| ATOM | 398 | O | LEU | A | 52 | 13.575 | 4.430 | 5.119 | 1.00 | 49.65 | O |
| ATOM | 399 | N | ARG | A | 53 | 14.710 | 3.759 | 6.946 | 1.00 | 47.79 | N |
| ATOM | 400 | CA | ARG | A | 53 | 13.516 | 3.318 | 7.660 | 1.00 | 52.73 | C |
| ATOM | 401 | CB | ARG | A | 53 | 13.917 | 2.467 | 8.867 | 1.00 | 51.48 | C |
| ATOM | 402 | CG | ARG | A | 53 | 14.100 | 0.984 | 8.561 | 1.00 | 51.16 | C |
| ATOM | 403 | CD | ARG | A | 53 | 14.589 | 0.191 | 9.771 | 1.00 | 45.71 | C |
| ATOM | 404 | NE | ARG | A | 53 | 15.916 | −0.388 | 9.578 | 1.00 | 42.81 | N |
| ATOM | 405 | CZ | ARG | A | 53 | 17.042 | 0.100 | 10.094 | 1.00 | 77.73 | C |
| ATOM | 406 | NH1 | ARG | A | 53 | 17.028 | 1.219 | 10.821 | 1.00 | 71.34 | N |
| ATOM | 407 | NH2 | ARG | A | 53 | 18.197 | −0.528 | 9.866 | 1.00 | 85.71 | N |
| ATOM | 408 | C | ARG | A | 53 | 12.687 | 4.510 | 8.141 | 1.00 | 56.39 | C |
| ATOM | 409 | O | ARG | A | 53 | 13.246 | 5.550 | 8.524 | 1.00 | 61.42 | O |
| ATOM | 410 | N | SER | A | 54 | 11.364 | 4.334 | 8.192 | 1.00 | 55.14 | N |
| ATOM | 411 | CA | SER | A | 54 | 10.467 | 5.348 | 8.762 | 1.00 | 49.04 | C |
| ATOM | 412 | CB | SER | A | 54 | 9.006 | 5.051 | 8.405 | 1.00 | 44.43 | C |
| ATOM | 413 | OG | SER | A | 54 | 8.613 | 3.772 | 8.875 | 1.00 | 43.44 | O |
| ATOM | 414 | C | SER | A | 54 | 10.624 | 5.522 | 10.273 | 1.00 | 48.50 | C |
| ATOM | 415 | O | SER | A | 54 | 10.259 | 6.556 | 10.822 | 1.00 | 51.17 | O |
| ATOM | 416 | N | GLU | A | 55 | 11.150 | 4.502 | 10.946 | 1.00 | 46.97 | N |
| ATOM | 417 | CA | GLU | A | 55 | 11.291 | 4.523 | 12.405 | 1.00 | 54.52 | C |
| ATOM | 418 | CB | GLU | A | 55 | 11.758 | 3.143 | 12.940 | 1.00 | 54.90 | C |
| ATOM | 419 | CG | GLU | A | 55 | 10.693 | 2.041 | 12.976 | 1.00 | 67.82 | C |
| ATOM | 420 | CD | GLU | A | 55 | 10.382 | 1.435 | 11.599 | 1.00 | 85.09 | C |
| ATOM | 421 | OE1 | GLU | A | 55 | 11.341 | 1.091 | 10.854 | 1.00 | 72.41 | O |
| ATOM | 422 | OE2 | GLU | A | 55 | 9.173 | 1.248 | 11.276 | 1.00 | 74.84 | O |
| ATOM | 423 | C | GLU | A | 55 | 12.327 | 5.582 | 12.754 | 1.00 | 52.06 | C |
| ATOM | 424 | O | GLU | A | 55 | 12.601 | 5.833 | 13.940 | 1.00 | 51.98 | O |
| ATOM | 425 | N | GLY | A | 56 | 12.989 | 6.098 | 11.715 | 1.00 | 48.38 | N |
| ATOM | 426 | CA | GLY | A | 56 | 14.208 | 6.864 | 11.937 | 1.00 | 51.87 | C |
| ATOM | 427 | C | GLY | A | 56 | 13.947 | 8.323 | 11.636 | 1.00 | 44.70 | C |
| ATOM | 428 | O | GLY | A | 56 | 14.541 | 9.217 | 12.241 | 1.00 | 43.70 | O |
| ATOM | 429 | N | PHE | A | 57 | 12.966 | 8.536 | 10.768 | 1.00 | 45.22 | N |
| ATOM | 430 | CA | PHE | A | 57 | 12.344 | 9.848 | 10.564 | 1.00 | 51.69 | C |
| ATOM | 431 | CB | PHE | A | 57 | 11.393 | 9.793 | 9.367 | 1.00 | 52.13 | C |
| ATOM | 432 | CG | PHE | A | 57 | 12.089 | 9.898 | 8.040 | 1.00 | 59.94 | C |
| ATOM | 433 | CD1 | PHE | A | 57 | 12.474 | 8.755 | 7.353 | 1.00 | 47.30 | C |
| ATOM | 434 | CE1 | PHE | A | 57 | 13.187 | 8.843 | 6.195 | 1.00 | 39.82 | C |
| ATOM | 435 | CZ | PHE | A | 57 | 13.525 | 10.082 | 5.694 | 1.00 | 62.27 | C |
| ATOM | 436 | CE2 | PHE | A | 57 | 13.187 | 11.232 | 6.385 | 1.00 | 64.63 | C |
| ATOM | 437 | CD2 | PHE | A | 57 | 12.462 | 11.135 | 7.542 | 1.00 | 69.80 | C |
| ATOM | 438 | C | PHE | A | 57 | 11.573 | 10.419 | 11.754 | 1.00 | 53.89 | C |
| ATOM | 439 | O | PHE | A | 57 | 10.747 | 9.739 | 12.371 | 1.00 | 62.31 | O |
| ATOM | 440 | N | ASP | A | 58 | 11.769 | 11.702 | 12.018 | 1.00 | 50.32 | N |
| ATOM | 441 | CA | ASP | A | 58 | 11.145 | 12.341 | 13.166 | 1.00 | 53.27 | C |
| ATOM | 442 | CB | ASP | A | 58 | 11.713 | 13.760 | 13.332 | 1.00 | 58.60 | C |
| ATOM | 443 | CG | ASP | A | 58 | 11.563 | 14.313 | 14.748 | 1.00 | 73.47 | C |
| ATOM | 444 | OD1 | ASP | A | 58 | 12.366 | 13.924 | 15.628 | 1.00 | 75.26 | O |
| ATOM | 445 | OD2 | ASP | A | 58 | 10.745 | 15.223 | 15.045 | 1.00 | 84.88 | O |
| ATOM | 446 | C | ASP | A | 58 | 9.623 | 12.355 | 12.986 | 1.00 | 46.77 | C |
| ATOM | 447 | O | ASP | A | 58 | 8.865 | 12.207 | 13.941 | 1.00 | 54.21 | O |
| ATOM | 448 | N | THR | A | 59 | 9.174 | 12.463 | 11.745 | 1.00 | 34.92 | N |
| ATOM | 449 | CA | THR | A | 59 | 7.745 | 12.495 | 11.476 | 1.00 | 36.86 | C |
| ATOM | 450 | CB | THR | A | 59 | 7.219 | 13.950 | 11.523 | 1.00 | 39.35 | C |
| ATOM | 451 | OG1 | THR | A | 59 | 7.396 | 14.468 | 12.845 | 1.00 | 47.05 | O |
| ATOM | 452 | CG2 | THR | A | 59 | 5.699 | 14.010 | 11.310 | 1.00 | 42.09 | C |
| ATOM | 453 | C | THR | A | 59 | 7.497 | 11.890 | 10.100 | 1.00 | 38.25 | C |
| ATOM | 454 | O | THR | A | 59 | 7.531 | 12.570 | 9.083 | 1.00 | 53.26 | O |
| ATOM | 455 | N | TYR | A | 60 | 7.174 | 10.616 | 10.061 | 1.00 | 33.69 | N |
| ATOM | 456 | CA | TYR | A | 60 | 6.901 | 9.989 | 8.787 | 1.00 | 38.93 | C |
| ATOM | 457 | CB | TYR | A | 60 | 7.589 | 8.623 | 8.767 | 1.00 | 42.08 | C |
| ATOM | 458 | CG | TYR | A | 60 | 7.845 | 8.093 | 7.381 | 1.00 | 55.45 | C |
| ATOM | 459 | CD1 | TYR | A | 60 | 9.036 | 8.357 | 6.727 | 1.00 | 68.19 | C |
| ATOM | 460 | CE1 | TYR | A | 60 | 9.248 | 7.909 | 5.435 | 1.00 | 72.80 | C |
| ATOM | 461 | CZ | TYR | A | 60 | 8.270 | 7.165 | 4.786 | 1.00 | 67.57 | C |
| ATOM | 462 | OH | TYR | A | 60 | 8.477 | 6.716 | 3.499 | 1.00 | 44.10 | O |
| ATOM | 463 | CE2 | TYR | A | 60 | 7.074 | 6.897 | 5.415 | 1.00 | 67.31 | C |
| ATOM | 464 | CD2 | TYR | A | 60 | 6.859 | 7.384 | 6.696 | 1.00 | 72.51 | C |
| ATOM | 465 | C | TYR | A | 60 | 5.390 | 9.858 | 8.518 | 1.00 | 41.58 | C |
| ATOM | 466 | O | TYR | A | 60 | 4.590 | 9.636 | 9.433 | 1.00 | 46.87 | O |
| ATOM | 467 | N | ARG | A | 61 | 4.997 | 10.010 | 7.260 | 1.00 | 39.32 | N |
| ATOM | 468 | CA | ARG | A | 61 | 3.594 | 9.964 | 6.906 | 1.00 | 48.77 | C |
| ATOM | 469 | CB | ARG | A | 61 | 2.914 | 11.316 | 7.176 | 1.00 | 52.74 | C |
| ATOM | 470 | CG | ARG | A | 61 | 1.385 | 11.277 | 7.170 | 1.00 | 60.53 | C |
| ATOM | 471 | CD | ARG | A | 61 | 0.722 | 12.639 | 7.313 | 1.00 | 59.14 | C |
| ATOM | 472 | NE | ARG | A | 61 | −0.151 | 12.892 | 6.173 | 1.00 | 77.69 | N |
| ATOM | 473 | CZ | ARG | A | 61 | −1.043 | 13.866 | 6.124 | 1.00 | 85.28 | C |
| ATOM | 474 | NH1 | ARG | A | 61 | −1.184 | 14.695 | 7.153 | 1.00 | 95.39 | N |
| ATOM | 475 | NH2 | ARG | A | 61 | −1.783 | 14.022 | 5.034 | 1.00 | 85.74 | N |
| ATOM | 476 | C | ARG | A | 61 | 3.495 | 9.633 | 5.432 | 1.00 | 58.51 | C |
| ATOM | 477 | O | ARG | A | 61 | 3.801 | 10.460 | 4.566 | 1.00 | 60.15 | O |
| ATOM | 478 | N | CYS | A | 62 | 3.022 | 8.428 | 5.150 | 1.00 | 67.48 | N |
| ATOM | 479 | CA | CYS | A | 62 | 2.764 | 8.047 | 3.775 | 1.00 | 71.37 | C |
| ATOM | 480 | CB | CYS | A | 62 | 3.825 | 7.090 | 3.270 | 1.00 | 77.84 | C |
| ATOM | 481 | SG | CYS | A | 62 | 3.838 | 7.107 | 1.458 | 1.00 | 89.93 | S |
| ATOM | 482 | C | CYS | A | 62 | 1.417 | 7.408 | 3.576 | 1.00 | 70.82 | C |
| ATOM | 483 | O | CYS | A | 62 | 1.106 | 6.366 | 4.146 | 1.00 | 68.04 | O |
| ATOM | 484 | N | ASP | A | 63 | 0.636 | 8.031 | 2.711 | 1.00 | 78.47 | N |
| ATOM | 485 | CA | ASP | A | 63 | −0.740 | 7.613 | 2.512 | 1.00 | 77.62 | C |
| ATOM | 486 | CB | ASP | A | 63 | −1.580 | 8.784 | 1.964 | 1.00 | 76.90 | C |
| ATOM | 487 | CG | ASP | A | 63 | −1.974 | 9.797 | 3.060 | 1.00 | 76.45 | C |
| ATOM | 488 | OD1 | ASP | A | 63 | −1.315 | 9.874 | 4.124 | 1.00 | 70.02 | O |
| ATOM | 489 | OD2 | ASP | A | 63 | −2.943 | 10.574 | 2.941 | 1.00 | 64.90 | O |
| ATOM | 490 | C | ASP | A | 63 | −0.702 | 6.421 | 1.554 | 1.00 | 74.37 | C |
| ATOM | 491 | O | ASP | A | 63 | −0.944 | 5.267 | 1.956 | 1.00 | 73.22 | O |
| ATOM | 492 | N | ARG | A | 64 | −0.299 | 6.688 | 0.314 | 1.00 | 70.58 | N |
| ATOM | 493 | CA | ARG | A | 64 | −0.155 | 5.620 | −0.667 | 1.00 | 72.81 | C |
| ATOM | 494 | CB | ARG | A | 64 | −0.893 | 5.967 | −1.971 | 1.00 | 72.93 | C |
| ATOM | 495 | CG | ARG | A | 64 | −1.406 | 7.410 | −2.035 | 1.00 | 82.01 | C |
| ATOM | 496 | CD | ARG | A | 64 | −2.655 | 7.594 | −2.927 | 1.00 | 101.60 | C |
| ATOM | 497 | NE | ARG | A | 64 | −3.887 | 7.830 | −2.161 | 1.00 | 96.51 | N |
| ATOM | 498 | CZ | ARG | A | 64 | −4.919 | 6.981 | −2.103 | 1.00 | 108.21 | C |
| ATOM | 499 | NH1 | ARG | A | 64 | −4.891 | 5.830 | −2.781 | 1.00 | 107.62 | N |
| ATOM | 500 | NH2 | ARG | A | 64 | −5.978 | 7.275 | −1.349 | 1.00 | 88.06 | N |
| ATOM | 501 | C | ARG | A | 64 | 1.318 | 5.298 | −0.947 | 1.00 | 73.05 | C |
| ATOM | 502 | O | ARG | A | 64 | 2.231 | 5.963 | −0.421 | 1.00 | 61.65 | O |
| ATOM | 503 | N | ASN | A | 65 | 1.528 | 4.272 | −1.776 | 1.00 | 73.91 | N |
| ATOM | 504 | CA | ASN | A | 65 | 2.845 | 3.978 | −2.339 | 1.00 | 68.69 | C |
| ATOM | 505 | CB | ASN | A | 65 | 2.922 | 2.553 | −2.920 | 1.00 | 63.03 | C |
| ATOM | 506 | CG | ASN | A | 65 | 2.956 | 1.469 | −1.836 | 1.00 | 69.46 | C |
| ATOM | 507 | OD1 | ASN | A | 65 | 2.717 | 0.284 | −2.112 | 1.00 | 69.07 | O |
| ATOM | 508 | ND2 | ASN | A | 65 | 3.223 | 1.880 | −0.590 | 1.00 | 52.17 | N |
| ATOM | 509 | C | ASN | A | 65 | 3.243 | 5.017 | −3.386 | 1.00 | 69.37 | C |
| ATOM | 510 | O | ASN | A | 65 | 2.393 | 5.543 | −4.122 | 1.00 | 57.38 | O |
| ATOM | 511 | N | LEU | A | 66 | 4.536 | 5.346 | −3.386 | 1.00 | 71.13 | N |
| ATOM | 512 | CA | LEU | A | 66 | 5.093 | 6.349 | −4.284 | 1.00 | 68.00 | C |
| ATOM | 513 | CB | LEU | A | 66 | 5.481 | 7.606 | −3.491 | 1.00 | 67.99 | C |
| ATOM | 514 | CG | LEU | A | 66 | 4.772 | 8.881 | −3.953 | 1.00 | 71.21 | C |
| ATOM | 515 | CD1 | LEU | A | 66 | 4.987 | 9.979 | −2.937 | 1.00 | 59.49 | C |
| ATOM | 516 | CD2 | LEU | A | 66 | 5.216 | 9.321 | −5.360 | 1.00 | 84.70 | C |
| ATOM | 517 | C | LEU | A | 66 | 6.296 | 5.790 | −5.055 | 1.00 | 59.04 | C |
| ATOM | 518 | O | LEU | A | 66 | 7.043 | 4.953 | −4.529 | 1.00 | 54.81 | O |
| ATOM | 519 | N | ALA | A | 67 | 6.484 | 6.268 | −6.286 | 1.00 | 47.72 | N |
| ATOM | 520 | CA | ALA | A | 67 | 7.684 | 5.972 | −7.062 | 1.00 | 44.10 | C |
| ATOM | 521 | CB | ALA | A | 67 | 7.408 | 4.828 | −8.060 | 1.00 | 31.16 | C |
| ATOM | 522 | C | ALA | A | 67 | 8.167 | 7.238 | −7.779 | 1.00 | 46.75 | C |
| ATOM | 523 | O | ALA | A | 67 | 7.638 | 7.606 | −8.830 | 1.00 | 47.71 | O |
| ATOM | 524 | N | MET | A | 68 | 9.191 | 7.888 | −7.218 | 1.00 | 49.92 | N |
| ATOM | 525 | CA | MET | A | 68 | 9.631 | 9.211 | −7.679 | 1.00 | 50.16 | C |
| ATOM | 526 | CB | MET | A | 68 | 9.782 | 10.166 | −6.492 | 1.00 | 49.13 | C |
| ATOM | 527 | CG | MET | A | 68 | 8.573 | 11.081 | −6.337 | 1.00 | 47.19 | C |
| ATOM | 528 | SD | MET | A | 68 | 8.994 | 12.411 | −5.264 | 1.00 | 73.48 | S |
| ATOM | 529 | CE | MET | A | 68 | 9.574 | 11.466 | −3.776 | 1.00 | 39.82 | C |
| ATOM | 530 | C | MET | A | 68 | 10.898 | 9.223 | −8.552 | 1.00 | 49.78 | C |
| ATOM | 531 | O | MET | A | 68 | 12.007 | 8.995 | −8.061 | 1.00 | 45.04 | O |
| ATOM | 532 | N | GLY | A | 69 | 10.725 | 9.500 | −9.843 | 1.00 | 49.41 | N |
| ATOM | 533 | CA | GLY | A | 69 | 11.856 | 9.617 | −10.739 | 1.00 | 49.30 | C |
| ATOM | 534 | C | GLY | A | 69 | 12.604 | 10.924 | −10.537 | 1.00 | 50.84 | C |
| ATOM | 535 | O | GLY | A | 69 | 12.231 | 11.972 | −11.079 | 1.00 | 43.53 | O |
| ATOM | 536 | N | VAL | A | 70 | 13.710 | 10.844 | −9.804 | 1.00 | 51.52 | N |
| ATOM | 537 | CA | VAL | A | 70 | 14.438 | 12.037 | −9.407 | 1.00 | 48.50 | C |
| ATOM | 538 | CB | VAL | A | 70 | 14.666 | 12.044 | −7.910 | 1.00 | 49.75 | C |
| ATOM | 539 | CG1 | VAL | A | 70 | 15.481 | 13.258 | −7.521 | 1.00 | 52.90 | C |
| ATOM | 540 | CG2 | VAL | A | 70 | 13.331 | 12.024 | −7.196 | 1.00 | 50.42 | C |
| ATOM | 541 | C | VAL | A | 70 | 15.793 | 12.106 | −10.075 | 1.00 | 44.71 | C |
| ATOM | 542 | O | VAL | A | 70 | 16.573 | 11.169 | −9.977 | 1.00 | 46.76 | O |
| ATOM | 543 | N | ASN | A | 71 | 16.082 | 13.231 | −10.719 | 1.00 | 43.92 | N |
| ATOM | 544 | CA | ASN | A | 71 | 17.444 | 13.552 | −11.137 | 1.00 | 38.77 | C |
| ATOM | 545 | CB | ASN | A | 71 | 17.446 | 14.698 | −12.132 | 1.00 | 32.62 | C |
| ATOM | 546 | CG | ASN | A | 71 | 18.837 | 14.997 | −12.622 | 1.00 | 47.55 | C |
| ATOM | 547 | OD1 | ASN | A | 71 | 19.766 | 14.215 | −12.397 | 1.00 | 58.36 | O |
| ATOM | 548 | ND2 | ASN | A | 71 | 18.967 | 16.056 | −13.409 | 1.00 | 53.95 | N |
| ATOM | 549 | C | ASN | A | 71 | 18.398 | 13.898 | −9.997 | 1.00 | 36.40 | C |
| ATOM | 550 | O | ASN | A | 71 | 18.171 | 14.859 | −9.254 | 1.00 | 41.68 | O |
| ATOM | 551 | N | LEU | A | 72 | 19.454 | 13.103 | −9.854 | 1.00 | 33.07 | N |
| ATOM | 552 | CA | LEU | A | 72 | 20.265 | 13.113 | −8.649 | 1.00 | 36.21 | C |
| ATOM | 553 | CB | LEU | A | 72 | 21.080 | 11.831 | −8.526 | 1.00 | 26.55 | C |
| ATOM | 554 | CG | LEU | A | 72 | 20.296 | 10.669 | −7.905 | 1.00 | 37.17 | C |
| ATOM | 555 | CD1 | LEU | A | 72 | 20.827 | 9.363 | −8.477 | 1.00 | 32.64 | C |
| ATOM | 556 | CD2 | LEU | A | 72 | 20.367 | 10.678 | −6.369 | 1.00 | 23.20 | C |
| ATOM | 557 | C | LEU | A | 72 | 21.169 | 14.333 | −8.665 | 1.00 | 41.36 | C |
| ATOM | 558 | O | LEU | A | 72 | 21.372 | 15.015 | −7.649 | 1.00 | 54.26 | O |
| ATOM | 559 | N | THR | A | 73 | 21.628 | 14.686 | −9.851 | 1.00 | 36.96 | N |
| ATOM | 560 | CA | THR | A | 73 | 22.509 | 15.828 | −9.945 | 1.00 | 36.11 | C |
| ATOM | 561 | CB | THR | A | 73 | 23.091 | 15.880 | −11.341 | 1.00 | 41.72 | C |
| ATOM | 562 | OG1 | THR | A | 73 | 23.682 | 14.601 | −11.619 | 1.00 | 58.03 | O |
| ATOM | 563 | CG2 | THR | A | 73 | 24.241 | 16.886 | −11.409 | 1.00 | 46.25 | C |
| ATOM | 564 | C | THR | A | 73 | 21.697 | 17.068 | −9.623 | 1.00 | 34.46 | C |
| ATOM | 565 | O | THR | A | 73 | 22.153 | 17.982 | −8.944 | 1.00 | 43.74 | O |
| ATOM | 566 | N | SER | A | 74 | 20.461 | 17.095 | −10.077 | 1.00 | 29.60 | N |
| ATOM | 567 | CA | SER | A | 74 | 19.658 | 18.258 | −9.798 | 1.00 | 33.88 | C |
| ATOM | 568 | CB | SER | A | 74 | 18.307 | 18.122 | −10.510 | 1.00 | 39.08 | C |
| ATOM | 569 | OG | SER | A | 74 | 18.488 | 18.312 | −11.902 | 1.00 | 51.38 | O |
| ATOM | 570 | C | SER | A | 74 | 19.487 | 18.333 | −8.288 | 1.00 | 33.48 | C |
| ATOM | 571 | O | SER | A | 74 | 19.606 | 19.403 | −7.659 | 1.00 | 28.94 | O |
| ATOM | 572 | N | MET | A | 75 | 19.132 | 17.181 | −7.724 | 1.00 | 34.88 | N |
| ATOM | 573 | CA | MET | A | 75 | 18.796 | 17.102 | −6.313 | 1.00 | 30.85 | C |
| ATOM | 574 | CB | MET | A | 75 | 18.339 | 15.698 | −5.936 | 1.00 | 24.42 | C |
| ATOM | 575 | CG | MET | A | 75 | 17.294 | 15.722 | −4.817 | 1.00 | 37.39 | C |
| ATOM | 576 | SD | MET | A | 75 | 16.784 | 14.106 | −4.228 | 1.00 | 31.65 | S |
| ATOM | 577 | CE | MET | A | 75 | 15.817 | 14.479 | −2.792 | 1.00 | 42.53 | C |
| ATOM | 578 | C | MET | A | 75 | 19.970 | 17.490 | −5.446 | 1.00 | 28.96 | C |
| ATOM | 579 | O | MET | A | 75 | 19.786 | 18.093 | −4.390 | 1.00 | 32.89 | O |
| ATOM | 580 | N | SER | A | 76 | 21.168 | 17.137 | −5.896 | 1.00 | 25.45 | N |
| ATOM | 581 | CA | SER | A | 76 | 22.385 | 17.402 | −5.142 | 1.00 | 30.95 | C |
| ATOM | 582 | CB | SER | A | 76 | 23.545 | 16.622 | −5.750 | 1.00 | 33.09 | C |
| ATOM | 583 | OG | SER | A | 76 | 24.118 | 17.332 | −6.845 | 1.00 | 54.60 | O |
| ATOM | 584 | C | SER | A | 76 | 22.726 | 18.892 | −5.147 | 1.00 | 35.54 | C |
| ATOM | 585 | O | SER | A | 76 | 23.334 | 19.419 | −4.209 | 1.00 | 35.32 | O |
| ATOM | 586 | N | LYS | A | 77 | 22.347 | 19.581 | −6.215 | 1.00 | 36.54 | N |
| ATOM | 587 | CA | LYS | A | 77 | 22.574 | 21.010 | −6.245 | 1.00 | 34.77 | C |
| ATOM | 588 | CB | LYS | A | 77 | 22.260 | 21.575 | −7.635 | 1.00 | 33.61 | C |
| ATOM | 589 | CG | LYS | A | 77 | 23.499 | 21.879 | −8.475 | 1.00 | 43.86 | C |
| ATOM | 590 | CD | LYS | A | 77 | 23.151 | 21.941 | −9.965 | 1.00 | 58.79 | C |
| ATOM | 591 | CE | LYS | A | 77 | 23.962 | 20.915 | −10.785 | 1.00 | 67.47 | C |
| ATOM | 592 | NZ | LYS | A | 77 | 23.607 | 20.863 | −12.245 | 1.00 | 53.59 | N |
| ATOM | 593 | C | LYS | A | 77 | 21.683 | 21.624 | −5.173 | 1.00 | 33.96 | C |
| ATOM | 594 | O | LYS | A | 77 | 22.110 | 22.529 | −4.433 | 1.00 | 42.28 | O |
| ATOM | 595 | N | ILE | A | 78 | 20.445 | 21.138 | −5.092 | 1.00 | 25.44 | N |
| ATOM | 596 | CA | ILE | A | 78 | 19.497 | 21.723 | −4.167 | 1.00 | 29.83 | C |
| ATOM | 597 | CB | ILE | A | 78 | 18.096 | 21.199 | −4.469 | 1.00 | 31.00 | C |
| ATOM | 598 | CG1 | ILE | A | 78 | 17.683 | 21.648 | −5.868 | 1.00 | 36.10 | C |
| ATOM | 599 | CD1 | ILE | A | 78 | 16.282 | 21.184 | −6.229 | 1.00 | 32.10 | C |
| ATOM | 600 | CG2 | ILE | A | 78 | 17.094 | 21.788 | −3.479 | 1.00 | 50.24 | C |
| ATOM | 601 | C | ILE | A | 78 | 19.922 | 21.435 | −2.724 | 1.00 | 27.25 | C |
| ATOM | 602 | O | ILE | A | 78 | 19.884 | 22.312 | −1.825 | 1.00 | 22.44 | O |
| ATOM | 603 | N | LEU | A | 79 | 20.303 | 20.182 | −2.505 | 1.00 | 24.54 | N |
| ATOM | 604 | CA | LEU | A | 79 | 20.627 | 19.737 | −1.167 | 1.00 | 26.77 | C |
| ATOM | 605 | CB | LEU | A | 79 | 20.682 | 18.223 | −1.094 | 1.00 | 15.75 | C |
| ATOM | 606 | CG | LEU | A | 79 | 19.572 | 17.631 | −0.227 | 1.00 | 36.18 | C |
| ATOM | 607 | CD1 | LEU | A | 79 | 18.471 | 17.000 | −1.069 | 1.00 | 54.60 | C |
| ATOM | 608 | CD2 | LEU | A | 79 | 20.150 | 16.633 | 0.774 | 1.00 | 57.28 | C |
| ATOM | 609 | C | LEU | A | 79 | 21.931 | 20.374 | −0.736 | 1.00 | 32.02 | C |
| ATOM | 610 | O | LEU | A | 79 | 22.341 | 20.271 | 0.421 | 1.00 | 36.77 | O |
| ATOM | 611 | N | LYS | A | 80 | 22.539 | 21.114 | −1.656 | 1.00 | 39.45 | N |
| ATOM | 612 | CA | LYS | A | 80 | 23.794 | 21.763 | −1.334 | 1.00 | 38.59 | C |
| ATOM | 613 | CB | LYS | A | 80 | 24.632 | 21.993 | −2.593 | 1.00 | 38.64 | C |
| ATOM | 614 | CG | LYS | A | 80 | 25.750 | 20.952 | −2.790 | 1.00 | 37.13 | C |
| ATOM | 615 | CD | LYS | A | 80 | 27.153 | 21.532 | −2.552 | 1.00 | 63.28 | C |
| ATOM | 616 | CE | LYS | A | 80 | 27.871 | 21.866 | −3.868 | 1.00 | 70.77 | C |
| ATOM | 617 | NZ | LYS | A | 80 | 27.939 | 23.347 | −4.163 | 1.00 | 30.00 | N |
| ATOM | 618 | C | LYS | A | 80 | 23.447 | 23.053 | −0.627 | 1.00 | 34.94 | C |
| ATOM | 619 | O | LYS | A | 80 | 24.293 | 23.680 | −0.002 | 1.00 | 36.58 | O |
| ATOM | 620 | N | CYS | A | 81 | 22.162 | 23.381 | −0.642 | 1.00 | 32.69 | N |
| ATOM | 621 | CA | CYS | A | 81 | 21.699 | 24.650 | −0.111 | 1.00 | 37.18 | C |
| ATOM | 622 | CB | CYS | A | 81 | 20.548 | 25.190 | −0.955 | 1.00 | 38.26 | C |
| ATOM | 623 | SG | CYS | A | 81 | 20.916 | 25.336 | −2.726 | 1.00 | 43.88 | S |
| ATOM | 624 | C | CYS | A | 81 | 21.219 | 24.498 | 1.317 | 1.00 | 38.64 | C |
| ATOM | 625 | O | CYS | A | 81 | 20.963 | 25.482 | 2.003 | 1.00 | 50.57 | O |
| ATOM | 626 | N | ALA | A | 82 | 21.089 | 23.264 | 1.774 | 1.00 | 35.92 | N |
| ATOM | 627 | CA | ALA | A | 82 | 20.740 | 23.050 | 3.166 | 1.00 | 30.34 | C |
| ATOM | 628 | CB | ALA | A | 82 | 20.035 | 21.717 | 3.315 | 1.00 | 31.64 | C |
| ATOM | 629 | C | ALA | A | 82 | 21.975 | 23.148 | 4.064 | 1.00 | 26.24 | C |
| ATOM | 630 | O | ALA | A | 82 | 23.083 | 22.867 | 3.618 | 1.00 | 28.22 | O |
| ATOM | 631 | N | GLY | A | 83 | 21.807 | 23.665 | 5.283 | 1.00 | 20.82 | N |
| ATOM | 632 | CA | GLY | A | 83 | 22.881 | 23.707 | 6.269 | 1.00 | 19.47 | C |
| ATOM | 633 | C | GLY | A | 83 | 23.033 | 22.412 | 7.058 | 1.00 | 33.96 | C |
| ATOM | 634 | O | GLY | A | 83 | 22.247 | 21.468 | 6.900 | 1.00 | 49.00 | O |
| ATOM | 635 | N | ASN | A | 84 | 24.095 | 22.305 | 7.843 | 1.00 | 39.60 | N |
| ATOM | 636 | CA | ASN | A | 84 | 24.412 | 21.024 | 8.465 | 1.00 | 49.05 | C |
| ATOM | 637 | CB | ASN | A | 84 | 25.831 | 21.025 | 9.035 | 1.00 | 58.27 | C |
| ATOM | 638 | CG | ASN | A | 84 | 26.779 | 20.145 | 8.226 | 1.00 | 75.52 | C |
| ATOM | 639 | OD1 | ASN | A | 84 | 27.081 | 19.011 | 8.619 | 1.00 | 92.71 | O |
| ATOM | 640 | ND2 | ASN | A | 84 | 27.208 | 20.644 | 7.064 | 1.00 | 59.69 | N |
| ATOM | 641 | C | ASN | A | 84 | 23.410 | 20.651 | 9.542 | 1.00 | 51.74 | C |
| ATOM | 642 | O | ASN | A | 84 | 23.016 | 19.490 | 9.690 | 1.00 | 58.98 | O |
| ATOM | 643 | N | GLU | A | 85 | 22.996 | 21.646 | 10.308 | 1.00 | 53.86 | N |
| ATOM | 644 | CA | GLU | A | 85 | 22.091 | 21.362 | 11.403 | 1.00 | 58.29 | C |
| ATOM | 645 | CB | GLU | A | 85 | 22.389 | 22.306 | 12.576 | 1.00 | 58.39 | C |
| ATOM | 646 | CG | GLU | A | 85 | 23.583 | 23.224 | 12.322 | 1.00 | 63.72 | C |
| ATOM | 647 | CD | GLU | A | 85 | 24.837 | 22.809 | 13.098 | 1.00 | 76.31 | C |
| ATOM | 648 | OE1 | GLU | A | 85 | 25.321 | 23.608 | 13.944 | 1.00 | 43.66 | O |
| ATOM | 649 | OE2 | GLU | A | 85 | 25.392 | 21.720 | 12.801 | 1.00 | 92.08 | O |
| ATOM | 650 | C | GLU | A | 85 | 20.630 | 21.430 | 10.912 | 1.00 | 54.13 | C |
| ATOM | 651 | O | GLU | A | 85 | 19.676 | 21.298 | 11.689 | 1.00 | 55.06 | O |
| ATOM | 652 | N | ASP | A | 86 | 20.467 | 21.689 | 9.618 | 1.00 | 48.11 | N |
| ATOM | 653 | CA | ASP | A | 86 | 19.193 | 22.203 | 9.116 | 1.00 | 45.63 | C |
| ATOM | 654 | CB | ASP | A | 86 | 19.324 | 22.775 | 7.691 | 1.00 | 40.38 | C |
| ATOM | 655 | CG | ASP | A | 86 | 19.682 | 24.268 | 7.671 | 1.00 | 58.04 | C |
| ATOM | 656 | OD1 | ASP | A | 86 | 20.587 | 24.698 | 8.434 | 1.00 | 70.85 | O |
| ATOM | 657 | OD2 | ASP | A | 86 | 19.125 | 25.084 | 6.894 | 1.00 | 71.39 | O |
| ATOM | 658 | C | ASP | A | 86 | 18.244 | 21.033 | 9.113 | 1.00 | 38.10 | C |
| ATOM | 659 | O | ASP | A | 86 | 18.617 | 19.942 | 8.709 | 1.00 | 40.58 | O |
| ATOM | 660 | N | ILE | A | 87 | 17.025 | 21.245 | 9.582 | 1.00 | 32.49 | N |
| ATOM | 661 | CA | ILE | A | 87 | 16.052 | 20.167 | 9.572 | 1.00 | 36.34 | C |
| ATOM | 662 | CB | ILE | A | 87 | 15.031 | 20.313 | 10.719 | 1.00 | 38.09 | C |
| ATOM | 663 | CG1 | ILE | A | 87 | 15.736 | 20.492 | 12.060 | 1.00 | 31.57 | C |
| ATOM | 664 | CD1 | ILE | A | 87 | 15.309 | 21.765 | 12.828 | 1.00 | 46.15 | C |
| ATOM | 665 | CG2 | ILE | A | 87 | 14.118 | 19.101 | 10.768 | 1.00 | 35.64 | C |
| ATOM | 666 | C | ILE | A | 87 | 15.353 | 20.012 | 8.216 | 1.00 | 32.33 | C |
| ATOM | 667 | O | ILE | A | 87 | 14.537 | 20.827 | 7.785 | 1.00 | 30.40 | O |
| ATOM | 668 | N | ILE | A | 88 | 15.676 | 18.938 | 7.528 | 1.00 | 22.21 | N |
| ATOM | 669 | CA | ILE | A | 88 | 15.226 | 18.841 | 6.159 | 1.00 | 34.72 | C |
| ATOM | 670 | CB | ILE | A | 88 | 16.282 | 18.093 | 5.318 | 1.00 | 35.56 | C |
| ATOM | 671 | CG1 | ILE | A | 88 | 17.585 | 18.907 | 5.292 | 1.00 | 45.50 | C |
| ATOM | 672 | CD1 | ILE | A | 88 | 18.632 | 18.386 | 4.328 | 1.00 | 53.27 | C |
| ATOM | 673 | CG2 | ILE | A | 88 | 15.760 | 17.768 | 3.917 | 1.00 | 37.07 | C |
| ATOM | 674 | C | ILE | A | 88 | 13.912 | 18.101 | 6.196 | 1.00 | 38.18 | C |
| ATOM | 675 | O | ILE | A | 88 | 13.765 | 17.104 | 6.899 | 1.00 | 52.67 | O |
| ATOM | 676 | N | THR | A | 89 | 12.952 | 18.570 | 5.423 | 1.00 | 38.81 | N |
| ATOM | 677 | CA | THR | A | 89 | 11.700 | 17.853 | 5.350 | 1.00 | 34.62 | C |
| ATOM | 678 | CB | THR | A | 89 | 10.607 | 18.727 | 5.999 | 1.00 | 39.26 | C |
| ATOM | 679 | OG1 | THR | A | 89 | 11.114 | 19.301 | 7.221 | 1.00 | 37.98 | O |
| ATOM | 680 | CG2 | THR | A | 89 | 9.416 | 17.875 | 6.428 | 1.00 | 20.55 | C |
| ATOM | 681 | C | THR | A | 89 | 11.397 | 17.641 | 3.881 | 1.00 | 35.32 | C |
| ATOM | 682 | O | THR | A | 89 | 11.376 | 18.619 | 3.115 | 1.00 | 42.23 | O |
| ATOM | 683 | N | LEU | A | 90 | 11.121 | 16.397 | 3.493 | 1.00 | 21.22 | N |
| ATOM | 684 | CA | LEU | A | 90 | 10.615 | 16.122 | 2.141 | 1.00 | 24.91 | C |
| ATOM | 685 | CB | LEU | A | 90 | 11.299 | 14.906 | 1.495 | 1.00 | 28.41 | C |
| ATOM | 686 | CG | LEU | A | 90 | 12.710 | 14.639 | 2.066 | 1.00 | 49.95 | C |
| ATOM | 687 | CD1 | LEU | A | 90 | 13.024 | 13.157 | 2.114 | 1.00 | 50.51 | C |
| ATOM | 688 | CD2 | LEU | A | 90 | 13.876 | 15.419 | 1.419 | 1.00 | 24.31 | C |
| ATOM | 689 | C | LEU | A | 90 | 9.120 | 15.912 | 2.138 | 1.00 | 29.53 | C |
| ATOM | 690 | O | LEU | A | 90 | 8.580 | 15.326 | 3.073 | 1.00 | 42.11 | O |
| ATOM | 691 | N | ARG | A | 91 | 8.449 | 16.385 | 1.087 | 1.00 | 32.79 | N |
| ATOM | 692 | CA | ARG | A | 91 | 6.981 | 16.405 | 1.052 | 1.00 | 35.93 | C |
| ATOM | 693 | CB | ARG | A | 91 | 6.447 | 17.676 | 1.756 | 1.00 | 43.18 | C |
| ATOM | 694 | CG | ARG | A | 91 | 5.056 | 17.575 | 2.366 | 1.00 | 52.85 | C |
| ATOM | 695 | CD | ARG | A | 91 | 4.511 | 18.896 | 2.890 | 1.00 | 70.57 | C |
| ATOM | 696 | NE | ARG | A | 91 | 4.471 | 19.938 | 1.863 | 1.00 | 76.36 | N |
| ATOM | 697 | CZ | ARG | A | 91 | 4.907 | 21.177 | 2.060 | 1.00 | 80.64 | C |
| ATOM | 698 | NH1 | ARG | A | 91 | 5.454 | 21.492 | 3.232 | 1.00 | 62.98 | N |
| ATOM | 699 | NH2 | ARG | A | 91 | 4.822 | 22.087 | 1.088 | 1.00 | 77.41 | N |
| ATOM | 700 | C | ARG | A | 91 | 6.468 | 16.325 | −0.400 | 1.00 | 35.95 | C |
| ATOM | 701 | O | ARG | A | 91 | 6.547 | 17.277 | −1.182 | 1.00 | 41.21 | O |
| ATOM | 702 | N | ALA | A | 92 | 5.942 | 15.171 | −0.769 | 1.00 | 39.14 | N |
| ATOM | 703 | CA | ALA | A | 92 | 5.283 | 15.021 | −2.059 | 1.00 | 41.05 | C |
| ATOM | 704 | CB | ALA | A | 92 | 5.808 | 13.807 | −2.769 | 1.00 | 29.04 | C |
| ATOM | 705 | C | ALA | A | 92 | 3.791 | 14.853 | −1.849 | 1.00 | 54.26 | C |
| ATOM | 706 | O | ALA | A | 92 | 3.348 | 14.453 | −0.765 | 1.00 | 56.99 | O |
| ATOM | 707 | N | GLU | A | 93 | 3.030 | 15.042 | −2.925 | 1.00 | 63.74 | N |
| ATOM | 708 | CA | GLU | A | 93 | 1.599 | 14.734 | −2.927 | 1.00 | 70.63 | C |
| ATOM | 709 | CB | GLU | A | 93 | 0.820 | 15.767 | −3.746 | 1.00 | 75.02 | C |
| ATOM | 710 | CG | GLU | A | 93 | 0.685 | 17.126 | −3.066 | 1.00 | 81.50 | C |
| ATOM | 711 | CD | GLU | A | 93 | −0.133 | 17.071 | −1.779 | 1.00 | 93.04 | C |
| ATOM | 712 | OE1 | GLU | A | 93 | −1.364 | 16.885 | −1.900 | 1.00 | 105.12 | O |
| ATOM | 713 | OE2 | GLU | A | 93 | 0.438 | 17.218 | −0.659 | 1.00 | 48.78 | O |
| ATOM | 714 | C | GLU | A | 93 | 1.377 | 13.347 | −3.504 | 1.00 | 76.23 | C |
| ATOM | 715 | O | GLU | A | 93 | 2.309 | 12.724 | −4.007 | 1.00 | 81.60 | O |
| ATOM | 716 | N | ASP | A | 94 | 0.146 | 12.855 | −3.458 | 1.00 | 82.27 | N |
| ATOM | 717 | CA | ASP | A | 94 | −0.083 | 11.478 | −3.884 | 1.00 | 82.86 | C |
| ATOM | 718 | CB | ASP | A | 94 | −1.289 | 10.880 | −3.159 | 1.00 | 82.77 | C |
| ATOM | 719 | CG | ASP | A | 94 | −1.121 | 10.952 | −1.642 | 1.00 | 93.07 | C |
| ATOM | 720 | OD1 | ASP | A | 94 | −0.064 | 10.508 | −1.126 | 1.00 | 67.03 | O |
| ATOM | 721 | OD2 | ASP | A | 94 | −1.963 | 11.492 | −0.887 | 1.00 | 102.26 | O |
| ATOM | 722 | C | ASP | A | 94 | −0.195 | 11.349 | −5.397 | 1.00 | 79.93 | C |
| ATOM | 723 | O | ASP | A | 94 | −0.024 | 10.272 | −5.958 | 1.00 | 75.26 | O |
| ATOM | 724 | N | ASN | A | 95 | −0.357 | 12.481 | −6.067 | 1.00 | 79.93 | N |
| ATOM | 725 | CA | ASN | A | 95 | −0.188 | 12.507 | −7.512 | 1.00 | 87.78 | C |
| ATOM | 726 | CB | ASN | A | 95 | −1.485 | 12.978 | −8.183 | 1.00 | 92.48 | C |
| ATOM | 727 | CG | ASN | A | 95 | −2.711 | 12.790 | −7.288 | 1.00 | 100.37 | C |
| ATOM | 728 | OD1 | ASN | A | 95 | −3.188 | 11.664 | −7.107 | 1.00 | 106.65 | O |
| ATOM | 729 | ND2 | ASN | A | 95 | −3.230 | 13.892 | −6.734 | 1.00 | 85.42 | N |
| ATOM | 730 | C | ASN | A | 95 | 0.961 | 13.426 | −7.912 | 1.00 | 92.52 | C |
| ATOM | 731 | O | ASN | A | 95 | 0.755 | 14.372 | −8.678 | 1.00 | 101.02 | O |
| ATOM | 732 | N | ALA | A | 96 | 2.153 | 13.185 | −7.363 | 1.00 | 92.03 | N |
| ATOM | 733 | CA | ALA | A | 96 | 3.228 | 14.183 | −7.399 | 1.00 | 83.37 | C |
| ATOM | 734 | CB | ALA | A | 96 | 4.307 | 13.858 | −6.388 | 1.00 | 84.40 | C |
| ATOM | 735 | C | ALA | A | 96 | 3.839 | 14.377 | −8.785 | 1.00 | 78.61 | C |
| ATOM | 736 | O | ALA | A | 96 | 4.300 | 13.432 | −9.421 | 1.00 | 76.40 | O |
| ATOM | 737 | N | ASP | A | 97 | 3.782 | 15.611 | −9.267 | 1.00 | 75.81 | N |
| ATOM | 738 | CA | ASP | A | 97 | 4.513 | 16.033 | −10.453 | 1.00 | 68.56 | C |
| ATOM | 739 | CB | ASP | A | 97 | 3.641 | 17.022 | −11.243 | 1.00 | 74.51 | C |
| ATOM | 740 | CG | ASP | A | 97 | 4.297 | 17.505 | −12.521 | 1.00 | 86.13 | C |
| ATOM | 741 | OD1 | ASP | A | 97 | 4.191 | 16.779 | −13.535 | 1.00 | 94.42 | O |
| ATOM | 742 | OD2 | ASP | A | 97 | 4.885 | 18.617 | −12.615 | 1.00 | 90.62 | O |
| ATOM | 743 | C | ASP | A | 97 | 5.831 | 16.693 | −10.013 | 1.00 | 59.52 | C |
| ATOM | 744 | O | ASP | A | 97 | 6.810 | 16.701 | −10.751 | 1.00 | 59.38 | O |
| ATOM | 745 | N | THR | A | 98 | 5.864 | 17.217 | −8.792 | 1.00 | 46.84 | N |
| ATOM | 746 | CA | THR | A | 98 | 7.077 | 17.790 | −8.237 | 1.00 | 38.26 | C |
| ATOM | 747 | CB | THR | A | 98 | 7.024 | 19.357 | −8.218 | 1.00 | 42.20 | C |
| ATOM | 748 | OG1 | THR | A | 98 | 5.799 | 19.813 | −7.641 | 1.00 | 33.19 | O |
| ATOM | 749 | CG2 | THR | A | 98 | 6.929 | 19.919 | −9.629 | 1.00 | 41.72 | C |
| ATOM | 750 | C | THR | A | 98 | 7.390 | 17.230 | −6.851 | 1.00 | 36.96 | C |
| ATOM | 751 | O | THR | A | 98 | 6.681 | 16.366 | −6.328 | 1.00 | 34.04 | O |
| ATOM | 752 | N | LEU | A | 99 | 8.478 | 17.710 | −6.260 | 1.00 | 34.99 | N |
| ATOM | 753 | CA | LEU | A | 99 | 8.848 | 17.301 | −4.913 | 1.00 | 40.24 | C |
| ATOM | 754 | CB | LEU | A | 99 | 9.884 | 16.183 | −4.940 | 1.00 | 42.67 | C |
| ATOM | 755 | CG | LEU | A | 99 | 10.526 | 15.934 | −3.579 | 1.00 | 47.69 | C |
| ATOM | 756 | CD1 | LEU | A | 99 | 9.507 | 15.264 | −2.676 | 1.00 | 51.69 | C |
| ATOM | 757 | CD2 | LEU | A | 99 | 11.795 | 15.096 | −3.712 | 1.00 | 44.30 | C |
| ATOM | 758 | C | LEU | A | 99 | 9.403 | 18.469 | −4.120 | 1.00 | 41.83 | C |
| ATOM | 759 | O | LEU | A | 99 | 10.318 | 19.182 | −4.561 | 1.00 | 44.01 | O |
| ATOM | 760 | N | ALA | A | 100 | 8.819 | 18.684 | −2.950 | 1.00 | 31.14 | N |
| ATOM | 761 | CA | ALA | A | 100 | 9.181 | 19.860 | −2.200 | 1.00 | 30.25 | C |
| ATOM | 762 | CB | ALA | A | 100 | 7.974 | 20.452 | −1.490 | 1.00 | 35.06 | C |
| ATOM | 763 | C | ALA | A | 100 | 10.266 | 19.483 | −1.220 | 1.00 | 36.02 | C |
| ATOM | 764 | O | ALA | A | 100 | 10.419 | 18.326 | −0.853 | 1.00 | 43.01 | O |
| ATOM | 765 | N | LEU | A | 101 | 11.046 | 20.473 | −0.817 | 1.00 | 34.01 | N |
| ATOM | 766 | CA | LEU | A | 101 | 12.162 | 20.215 | 0.059 | 1.00 | 17.63 | C |
| ATOM | 767 | CB | LEU | A | 101 | 13.416 | 20.141 | −0.788 | 1.00 | 22.15 | C |
| ATOM | 768 | CG | LEU | A | 101 | 13.826 | 18.762 | −1.330 | 1.00 | 26.07 | C |
| ATOM | 769 | CD1 | LEU | A | 101 | 14.135 | 18.912 | −2.804 | 1.00 | 9.40 | C |
| ATOM | 770 | CD2 | LEU | A | 101 | 15.060 | 18.242 | −0.602 | 1.00 | 26.11 | C |
| ATOM | 771 | C | LEU | A | 101 | 12.266 | 21.350 | 1.045 | 1.00 | 22.78 | C |
| ATOM | 772 | O | LEU | A | 101 | 12.527 | 22.503 | 0.675 | 1.00 | 30.42 | O |
| ATOM | 773 | N | VAL | A | 102 | 11.947 | 21.061 | 2.295 | 1.00 | 23.80 | N |
| ATOM | 774 | CA | VAL | A | 102 | 11.929 | 22.128 | 3.286 | 1.00 | 30.16 | C |
| ATOM | 775 | CB | VAL | A | 102 | 10.603 | 22.122 | 4.102 | 1.00 | 31.86 | C |
| ATOM | 776 | CG1 | VAL | A | 102 | 10.471 | 23.411 | 4.954 | 1.00 | 39.09 | C |
| ATOM | 777 | CG2 | VAL | A | 102 | 9.388 | 21.995 | 3.162 | 1.00 | 25.37 | C |
| ATOM | 778 | C | VAL | A | 102 | 13.186 | 22.133 | 4.181 | 1.00 | 30.77 | C |
| ATOM | 779 | O | VAL | A | 102 | 13.453 | 21.153 | 4.890 | 1.00 | 35.99 | O |
| ATOM | 780 | N | PHE | A | 103 | 13.986 | 23.201 | 4.099 | 1.00 | 24.97 | N |
| ATOM | 781 | CA | PHE | A | 103 | 15.209 | 23.342 | 4.908 | 1.00 | 23.75 | C |
| ATOM | 782 | CB | PHE | A | 103 | 16.436 | 23.820 | 4.098 | 1.00 | 15.90 | C |
| ATOM | 783 | CG | PHE | A | 103 | 16.792 | 22.932 | 2.944 | 1.00 | 27.10 | C |
| ATOM | 784 | CD1 | PHE | A | 103 | 16.478 | 21.582 | 2.967 | 1.00 | 51.51 | C |
| ATOM | 785 | CE1 | PHE | A | 103 | 16.745 | 20.766 | 1.878 | 1.00 | 37.18 | C |
| ATOM | 786 | CZ | PHE | A | 103 | 17.328 | 21.294 | 0.761 | 1.00 | 44.69 | C |
| ATOM | 787 | CE2 | PHE | A | 103 | 17.662 | 22.637 | 0.730 | 1.00 | 55.77 | C |
| ATOM | 788 | CD2 | PHE | A | 103 | 17.382 | 23.451 | 1.812 | 1.00 | 43.28 | C |
| ATOM | 789 | C | PHE | A | 103 | 14.957 | 24.343 | 6.025 | 1.00 | 27.33 | C |
| ATOM | 790 | O | PHE | A | 103 | 14.902 | 25.549 | 5.805 | 1.00 | 21.39 | O |
| ATOM | 791 | N | GLU | A | 104 | 14.839 | 23.831 | 7.244 | 1.00 | 37.59 | N |
| ATOM | 792 | CA | GLU | A | 104 | 14.418 | 24.653 | 8.377 | 1.00 | 40.58 | C |
| ATOM | 793 | CB | GLU | A | 104 | 13.272 | 23.967 | 9.131 | 1.00 | 46.33 | C |
| ATOM | 794 | CG | GLU | A | 104 | 12.445 | 24.849 | 10.049 | 1.00 | 30.15 | C |
| ATOM | 795 | CD | GLU | A | 104 | 11.403 | 24.018 | 10.765 | 1.00 | 47.95 | C |
| ATOM | 796 | OE1 | GLU | A | 104 | 11.616 | 23.763 | 11.970 | 1.00 | 62.54 | O |
| ATOM | 797 | OE2 | GLU | A | 104 | 10.437 | 23.549 | 10.106 | 1.00 | 35.15 | O |
| ATOM | 798 | C | GLU | A | 104 | 15.599 | 24.774 | 9.302 | 1.00 | 41.22 | C |
| ATOM | 799 | O | GLU | A | 104 | 16.205 | 23.770 | 9.669 | 1.00 | 47.88 | O |
| ATOM | 800 | N | ALA | A | 105 | 15.929 | 26.006 | 9.656 | 1.00 | 41.22 | N |
| ATOM | 801 | CA | ALA | A | 105 | 16.962 | 26.265 | 10.642 | 1.00 | 43.97 | C |
| ATOM | 802 | CB | ALA | A | 105 | 17.326 | 27.736 | 10.649 | 1.00 | 43.39 | C |
| ATOM | 803 | C | ALA | A | 105 | 16.443 | 25.841 | 12.004 | 1.00 | 52.93 | C |
| ATOM | 804 | O | ALA | A | 105 | 15.239 | 25.887 | 12.258 | 1.00 | 49.28 | O |
| ATOM | 805 | N | PRO | A | 106 | 17.346 | 25.374 | 12.863 | 1.00 | 62.11 | N |
| ATOM | 806 | CA | PRO | A | 106 | 16.954 | 24.934 | 14.203 | 1.00 | 64.88 | C |
| ATOM | 807 | CB | PRO | A | 106 | 18.292 | 24.850 | 14.941 | 1.00 | 63.32 | C |
| ATOM | 808 | CG | PRO | A | 106 | 19.251 | 24.464 | 13.866 | 1.00 | 57.14 | C |
| ATOM | 809 | CD | PRO | A | 106 | 18.797 | 25.236 | 12.641 | 1.00 | 63.64 | C |
| ATOM | 810 | C | PRO | A | 106 | 16.024 | 25.977 | 14.819 | 1.00 | 64.41 | C |
| ATOM | 811 | O | PRO | A | 106 | 14.867 | 25.654 | 15.133 | 1.00 | 64.47 | O |
| ATOM | 812 | N | ASN | A | 107 | 16.480 | 27.227 | 14.891 | 1.00 | 65.58 | N |
| ATOM | 813 | CA | ASN | A | 107 | 15.716 | 28.263 | 15.585 | 1.00 | 64.53 | C |
| ATOM | 814 | CB | ASN | A | 107 | 16.582 | 29.461 | 15.961 | 1.00 | 62.17 | C |
| ATOM | 815 | CG | ASN | A | 107 | 17.147 | 30.156 | 14.758 | 1.00 | 65.25 | C |
| ATOM | 816 | OD1 | ASN | A | 107 | 17.737 | 29.528 | 13.886 | 1.00 | 96.15 | O |
| ATOM | 817 | ND2 | ASN | A | 107 | 17.011 | 31.462 | 14.720 | 1.00 | 59.73 | N |
| ATOM | 818 | C | ASN | A | 107 | 14.576 | 28.740 | 14.732 | 1.00 | 61.48 | C |
| ATOM | 819 | O | ASN | A | 107 | 13.818 | 29.596 | 15.146 | 1.00 | 71.49 | O |
| ATOM | 820 | N | GLN | A | 108 | 14.479 | 28.185 | 13.537 | 1.00 | 55.94 | N |
| ATOM | 821 | CA | GLN | A | 108 | 13.316 | 28.363 | 12.695 | 1.00 | 49.91 | C |
| ATOM | 822 | CB | GLN | A | 108 | 12.069 | 27.971 | 13.473 | 1.00 | 54.15 | C |
| ATOM | 823 | CG | GLN | A | 108 | 10.970 | 27.416 | 12.613 | 1.00 | 66.91 | C |
| ATOM | 824 | CD | GLN | A | 108 | 9.864 | 26.790 | 13.425 | 1.00 | 74.47 | C |
| ATOM | 825 | OE1 | GLN | A | 108 | 8.851 | 26.349 | 12.864 | 1.00 | 78.00 | O |
| ATOM | 826 | NE2 | GLN | A | 108 | 10.059 | 26.723 | 14.746 | 1.00 | 78.46 | N |
| ATOM | 827 | C | GLN | A | 108 | 13.214 | 29.783 | 12.140 | 1.00 | 50.95 | C |
| ATOM | 828 | O | GLN | A | 108 | 12.129 | 30.258 | 11.815 | 1.00 | 54.13 | O |
| ATOM | 829 | N | GLU | A | 109 | 14.344 | 30.468 | 12.001 | 1.00 | 47.69 | N |
| ATOM | 830 | CA | GLU | A | 109 | 14.333 | 31.804 | 11.410 | 1.00 | 41.38 | C |
| ATOM | 831 | CB | GLU | A | 109 | 15.296 | 32.718 | 12.166 | 1.00 | 37.46 | C |
| ATOM | 832 | CG | GLU | A | 109 | 15.638 | 34.009 | 11.420 | 1.00 | 72.87 | C |
| ATOM | 833 | CD | GLU | A | 109 | 16.497 | 34.954 | 12.264 | 1.00 | 102.10 | C |
| ATOM | 834 | OE1 | GLU | A | 109 | 16.375 | 34.869 | 13.517 | 1.00 | 106.48 | O |
| ATOM | 835 | OE2 | GLU | A | 109 | 17.300 | 35.756 | 11.693 | 1.00 | 86.52 | O |
| ATOM | 836 | C | GLU | A | 109 | 14.635 | 31.814 | 9.906 | 1.00 | 35.76 | C |
| ATOM | 837 | O | GLU | A | 109 | 13.976 | 32.486 | 9.142 | 1.00 | 42.67 | O |
| ATOM | 838 | N | LYS | A | 110 | 15.577 | 31.010 | 9.455 | 1.00 | 28.10 | N |
| ATOM | 839 | CA | LYS | A | 110 | 15.774 | 30.874 | 8.035 | 1.00 | 31.47 | C |
| ATOM | 840 | CB | LYS | A | 110 | 17.282 | 30.775 | 7.761 | 1.00 | 32.85 | C |
| ATOM | 841 | CG | LYS | A | 110 | 17.784 | 31.130 | 6.361 | 1.00 | 40.78 | C |
| ATOM | 842 | CD | LYS | A | 110 | 19.076 | 30.329 | 6.059 | 1.00 | 55.40 | C |
| ATOM | 843 | CE | LYS | A | 110 | 18.875 | 29.252 | 4.976 | 1.00 | 58.67 | C |
| ATOM | 844 | NZ | LYS | A | 110 | 19.301 | 27.873 | 5.364 | 1.00 | 64.23 | N |
| ATOM | 845 | C | LYS | A | 110 | 14.993 | 29.616 | 7.620 | 1.00 | 32.35 | C |
| ATOM | 846 | O | LYS | A | 110 | 15.431 | 28.481 | 7.805 | 1.00 | 36.53 | O |
| ATOM | 847 | N | VAL | A | 111 | 13.818 | 29.815 | 7.046 | 1.00 | 33.28 | N |
| ATOM | 848 | CA | VAL | A | 111 | 13.106 | 28.705 | 6.433 | 1.00 | 34.14 | C |
| ATOM | 849 | CB | VAL | A | 111 | 11.665 | 28.661 | 6.902 | 1.00 | 32.28 | C |
| ATOM | 850 | CG1 | VAL | A | 111 | 10.959 | 27.444 | 6.333 | 1.00 | 21.73 | C |
| ATOM | 851 | CG2 | VAL | A | 111 | 11.619 | 28.706 | 8.415 | 1.00 | 36.41 | C |
| ATOM | 852 | C | VAL | A | 111 | 13.133 | 28.725 | 4.904 | 1.00 | 35.53 | C |
| ATOM | 853 | O | VAL | A | 111 | 12.721 | 29.694 | 4.285 | 1.00 | 29.41 | O |
| ATOM | 854 | N | SER | A | 112 | 13.611 | 27.640 | 4.301 | 1.00 | 39.12 | N |
| ATOM | 855 | CA | SER | A | 112 | 13.822 | 27.596 | 2.861 | 1.00 | 39.45 | C |
| ATOM | 856 | CB | SER | A | 112 | 15.291 | 27.314 | 2.548 | 1.00 | 36.93 | C |
| ATOM | 857 | OG | SER | A | 112 | 16.148 | 28.044 | 3.415 | 1.00 | 37.18 | O |
| ATOM | 858 | C | SER | A | 112 | 12.968 | 26.501 | 2.232 | 1.00 | 43.11 | C |
| ATOM | 859 | O | SER | A | 112 | 12.891 | 25.392 | 2.751 | 1.00 | 39.69 | O |
| ATOM | 860 | N | ASP | A | 113 | 12.331 | 26.810 | 1.107 | 1.00 | 43.36 | N |
| ATOM | 861 | CA | ASP | A | 113 | 11.273 | 25.948 | 0.593 | 1.00 | 38.57 | C |
| ATOM | 862 | CB | ASP | A | 113 | 9.906 | 26.618 | 0.749 | 1.00 | 42.08 | C |
| ATOM | 863 | CG | ASP | A | 113 | 8.769 | 25.647 | 0.500 | 1.00 | 68.04 | C |
| ATOM | 864 | OD1 | ASP | A | 113 | 7.795 | 25.601 | 1.310 | 1.00 | 76.97 | O |
| ATOM | 865 | OD2 | ASP | A | 113 | 8.850 | 24.808 | −0.432 | 1.00 | 88.98 | O |
| ATOM | 866 | C | ASP | A | 113 | 11.505 | 25.689 | −0.862 | 1.00 | 30.35 | C |
| ATOM | 867 | O | ASP | A | 113 | 11.113 | 26.486 | −1.700 | 1.00 | 44.04 | O |
| ATOM | 868 | N | TYR | A | 114 | 12.192 | 24.605 | −1.170 | 1.00 | 27.25 | N |
| ATOM | 869 | CA | TYR | A | 114 | 12.633 | 24.354 | −2.547 | 1.00 | 30.10 | C |
| ATOM | 870 | CB | TYR | A | 114 | 14.047 | 23.759 | −2.581 | 1.00 | 25.11 | C |
| ATOM | 871 | CG | TYR | A | 114 | 15.139 | 24.757 | −2.254 | 1.00 | 27.97 | C |
| ATOM | 872 | CD1 | TYR | A | 114 | 15.707 | 25.542 | −3.246 | 1.00 | 24.83 | C |
| ATOM | 873 | CE1 | TYR | A | 114 | 16.638 | 26.500 | −2.949 | 1.00 | 9.67 | C |
| ATOM | 874 | CZ | TYR | A | 114 | 17.006 | 26.707 | −1.633 | 1.00 | 32.31 | C |
| ATOM | 875 | OH | TYR | A | 114 | 18.000 | 27.599 | −1.294 | 1.00 | 33.82 | O |
| ATOM | 876 | CE2 | TYR | A | 114 | 16.436 | 25.979 | −0.631 | 1.00 | 24.78 | C |
| ATOM | 877 | CD2 | TYR | A | 114 | 15.496 | 25.020 | −0.941 | 1.00 | 29.22 | C |
| ATOM | 878 | C | TYR | A | 114 | 11.654 | 23.431 | −3.259 | 1.00 | 28.17 | C |
| ATOM | 879 | O | TYR | A | 114 | 10.852 | 22.770 | −2.613 | 1.00 | 30.85 | O |
| ATOM | 880 | N | GLU | A | 115 | 11.648 | 23.450 | −4.585 | 1.00 | 24.73 | N |
| ATOM | 881 | CA | GLU | A | 115 | 10.654 | 22.687 | −5.318 | 1.00 | 22.16 | C |
| ATOM | 882 | CB | GLU | A | 115 | 9.501 | 23.577 | −5.737 | 1.00 | 22.66 | C |
| ATOM | 883 | CG | GLU | A | 115 | 8.287 | 22.790 | −6.188 | 1.00 | 55.43 | C |
| ATOM | 884 | CD | GLU | A | 115 | 6.994 | 23.507 | −5.861 | 1.00 | 84.89 | C |
| ATOM | 885 | OE1 | GLU | A | 115 | 6.853 | 24.673 | −6.292 | 1.00 | 78.83 | O |
| ATOM | 886 | OE2 | GLU | A | 115 | 6.121 | 22.906 | −5.190 | 1.00 | 101.80 | O |
| ATOM | 887 | C | GLU | A | 115 | 11.279 | 22.149 | −6.569 | 1.00 | 30.89 | C |
| ATOM | 888 | O | GLU | A | 115 | 11.661 | 22.909 | −7.477 | 1.00 | 43.05 | O |
| ATOM | 889 | N | MET | A | 116 | 11.369 | 20.830 | −6.632 | 1.00 | 31.32 | N |
| ATOM | 890 | CA | MET | A | 116 | 12.107 | 20.200 | −7.719 | 1.00 | 32.06 | C |
| ATOM | 891 | CB | MET | A | 116 | 13.215 | 19.299 | −7.160 | 1.00 | 35.48 | C |
| ATOM | 892 | CG | MET | A | 116 | 14.156 | 18.703 | −8.205 | 1.00 | 50.71 | C |
| ATOM | 893 | SD | MET | A | 116 | 15.187 | 17.368 | −7.538 | 1.00 | 50.56 | S |
| ATOM | 894 | CE | MET | A | 116 | 14.065 | 16.596 | −6.441 | 1.00 | 59.76 | C |
| ATOM | 895 | C | MET | A | 116 | 11.175 | 19.415 | −8.622 | 1.00 | 22.81 | C |
| ATOM | 896 | O | MET | A | 116 | 10.463 | 18.533 | −8.156 | 1.00 | 25.60 | O |
| ATOM | 897 | N | LYS | A | 117 | 11.233 | 19.681 | −9.922 | 1.00 | 34.44 | N |
| ATOM | 898 | CA | LYS | A | 117 | 10.438 | 18.909 | −10.879 | 1.00 | 44.12 | C |
| ATOM | 899 | CB | LYS | A | 117 | 10.501 | 19.551 | −12.270 | 1.00 | 37.71 | C |
| ATOM | 900 | CG | LYS | A | 117 | 9.458 | 20.638 | −12.480 | 1.00 | 54.23 | C |
| ATOM | 901 | CD | LYS | A | 117 | 10.102 | 22.014 | −12.373 | 1.00 | 64.34 | C |
| ATOM | 902 | CE | LYS | A | 117 | 9.912 | 22.787 | −13.677 | 1.00 | 76.74 | C |
| ATOM | 903 | NZ | LYS | A | 117 | 11.181 | 22.939 | −14.476 | 1.00 | 57.08 | N |
| ATOM | 904 | C | LYS | A | 117 | 10.881 | 17.442 | −10.937 | 1.00 | 42.18 | C |
| ATOM | 905 | O | LYS | A | 117 | 12.058 | 17.170 | −11.101 | 1.00 | 55.64 | O |
| ATOM | 906 | N | LEU | A | 118 | 9.951 | 16.503 | −10.829 | 1.00 | 33.57 | N |
| ATOM | 907 | CA | LEU | A | 118 | 10.239 | 15.119 | −11.178 | 1.00 | 35.62 | C |
| ATOM | 908 | CB | LEU | A | 118 | 9.191 | 14.200 | −10.559 | 1.00 | 32.81 | C |
| ATOM | 909 | CG | LEU | A | 118 | 8.880 | 14.551 | −9.108 | 1.00 | 28.27 | C |
| ATOM | 910 | CD1 | LEU | A | 118 | 7.778 | 13.636 | −8.654 | 1.00 | 36.41 | C |
| ATOM | 911 | CD2 | LEU | A | 118 | 10.100 | 14.366 | −8.193 | 1.00 | 50.57 | C |
| ATOM | 912 | C | LEU | A | 118 | 10.335 | 14.849 | −12.678 | 1.00 | 42.59 | C |
| ATOM | 913 | O | LEU | A | 118 | 10.126 | 15.751 | −13.500 | 1.00 | 45.71 | O |
| ATOM | 914 | N | MET | A | 119 | 10.615 | 13.588 | −13.017 | 1.00 | 48.98 | N |
| ATOM | 915 | CA | MET | A | 119 | 10.615 | 13.124 | −14.404 | 1.00 | 46.03 | C |
| ATOM | 916 | CB | MET | A | 119 | 11.971 | 13.372 | −15.062 | 1.00 | 56.50 | C |
| ATOM | 917 | CG | MET | A | 119 | 13.165 | 12.835 | −14.273 | 1.00 | 65.08 | C |
| ATOM | 918 | SD | MET | A | 119 | 14.599 | 12.625 | −15.376 | 1.00 | 91.74 | S |
| ATOM | 919 | CE | MET | A | 119 | 15.176 | 14.364 | −15.622 | 1.00 | 51.92 | C |
| ATOM | 920 | C | MET | A | 119 | 10.206 | 11.666 | −14.562 | 1.00 | 38.33 | C |
| ATOM | 921 | O | MET | A | 119 | 10.297 | 10.866 | −13.628 | 1.00 | 28.42 | O |
| ATOM | 922 | N | ASP | A | 120 | 9.738 | 11.327 | −15.759 | 1.00 | 48.15 | N |
| ATOM | 923 | CA | ASP | A | 120 | 9.292 | 9.962 | −16.007 | 1.00 | 59.34 | C |
| ATOM | 924 | CB | ASP | A | 120 | 8.249 | 9.896 | −17.124 | 1.00 | 52.89 | C |
| ATOM | 925 | CG | ASP | A | 120 | 7.058 | 9.032 | −16.750 | 1.00 | 68.14 | C |
| ATOM | 926 | OD1 | ASP | A | 120 | 7.179 | 8.220 | −15.798 | 1.00 | 59.26 | O |
| ATOM | 927 | OD2 | ASP | A | 120 | 5.982 | 9.066 | −17.390 | 1.00 | 81.85 | O |
| ATOM | 928 | C | ASP | A | 120 | 10.486 | 9.078 | −16.325 | 1.00 | 64.03 | C |
| ATOM | 929 | O | ASP | A | 120 | 11.425 | 9.515 | −17.010 | 1.00 | 61.71 | O |
| ATOM | 930 | N | LEU | A | 121 | 10.483 | 7.876 | −15.750 | 1.00 | 66.79 | N |
| ATOM | 931 | CA | LEU | A | 121 | 11.626 | 6.978 | −15.855 | 1.00 | 71.15 | C |
| ATOM | 932 | CB | LEU | A | 121 | 12.343 | 6.827 | −14.508 | 1.00 | 73.97 | C |
| ATOM | 933 | CG | LEU | A | 121 | 13.334 | 7.919 | −14.057 | 1.00 | 70.90 | C |
| ATOM | 934 | CD1 | LEU | A | 121 | 14.237 | 7.383 | −12.956 | 1.00 | 37.36 | C |
| ATOM | 935 | CD2 | LEU | A | 121 | 14.157 | 8.527 | −15.205 | 1.00 | 63.87 | C |
| ATOM | 936 | C | LEU | A | 121 | 11.236 | 5.617 | −16.409 | 1.00 | 72.48 | C |
| ATOM | 937 | O | LEU | A | 121 | 10.317 | 4.967 | −15.916 | 1.00 | 65.30 | O |
| ATOM | 938 | N | ASP | A | 122 | 11.942 | 5.216 | −17.460 | 1.00 | 81.66 | N |
| ATOM | 939 | CA | ASP | A | 122 | 11.750 | 3.912 | −18.080 | 1.00 | 89.51 | C |
| ATOM | 940 | CB | ASP | A | 122 | 12.133 | 4.007 | −19.558 | 1.00 | 92.57 | C |
| ATOM | 941 | CG | ASP | A | 122 | 11.359 | 3.046 | −20.408 | 1.00 | 91.83 | C |
| ATOM | 942 | OD1 | ASP | A | 122 | 11.748 | 1.857 | −20.442 | 1.00 | 94.45 | O |
| ATOM | 943 | OD2 | ASP | A | 122 | 10.321 | 3.386 | −21.013 | 1.00 | 81.41 | O |
| ATOM | 944 | C | ASP | A | 122 | 12.607 | 2.849 | −17.390 | 1.00 | 93.04 | C |
| ATOM | 945 | O | ASP | A | 122 | 13.523 | 2.285 | −18.003 | 1.00 | 95.67 | O |
| ATOM | 946 | N | VAL | A | 123 | 12.329 | 2.610 | −16.109 | 1.00 | 92.14 | N |
| ATOM | 947 | CA | VAL | A | 123 | 13.312 | 2.019 | −15.197 | 1.00 | 88.86 | C |
| ATOM | 948 | CB | VAL | A | 123 | 13.020 | 2.360 | −13.712 | 1.00 | 93.79 | C |
| ATOM | 949 | CG1 | VAL | A | 123 | 11.891 | 1.459 | −13.166 | 1.00 | 79.84 | C |
| ATOM | 950 | CG2 | VAL | A | 123 | 14.316 | 2.264 | −12.855 | 1.00 | 87.35 | C |
| ATOM | 951 | C | VAL | A | 123 | 13.275 | 0.508 | −15.327 | 1.00 | 87.13 | C |
| ATOM | 952 | O | VAL | A | 123 | 12.200 | −0.069 | −15.485 | 1.00 | 87.16 | O |
| ATOM | 953 | N | GLU | A | 124 | 14.450 | −0.116 | −15.252 | 1.00 | 89.30 | N |
| ATOM | 954 | CA | GLU | A | 124 | 14.555 | −1.563 | −15.145 | 1.00 | 93.70 | C |
| ATOM | 955 | CB | GLU | A | 124 | 15.679 | −2.088 | −16.048 | 1.00 | 93.48 | C |
| ATOM | 956 | CG | GLU | A | 124 | 15.729 | −3.608 | −16.165 | 1.00 | 95.55 | C |
| ATOM | 957 | CD | GLU | A | 124 | 17.081 | −4.120 | −16.637 | 1.00 | 93.87 | C |
| ATOM | 958 | OE1 | GLU | A | 124 | 18.103 | −3.756 | −16.015 | 1.00 | 70.38 | O |
| ATOM | 959 | OE2 | GLU | A | 124 | 17.122 | −4.896 | −17.622 | 1.00 | 87.21 | O |
| ATOM | 960 | C | GLU | A | 124 | 14.824 | −1.947 | −13.697 | 1.00 | 94.29 | C |
| ATOM | 961 | O | GLU | A | 124 | 15.909 | −1.677 | −13.176 | 1.00 | 99.31 | O |
| ATOM | 962 | N | GLN | A | 125 | 13.851 | −2.613 | −13.074 | 1.00 | 93.26 | N |
| ATOM | 963 | CA | GLN | A | 125 | 13.952 | −3.043 | −11.673 | 1.00 | 92.22 | C |
| ATOM | 964 | CB | GLN | A | 125 | 12.562 | −3.039 | −11.012 | 1.00 | 94.54 | C |
| ATOM | 965 | CG | GLN | A | 125 | 11.910 | −1.647 | −10.891 | 1.00 | 95.74 | C |
| ATOM | 966 | CD | GLN | A | 125 | 12.767 | −0.653 | −10.107 | 1.00 | 104.11 | C |
| ATOM | 967 | OE1 | GLN | A | 125 | 13.260 | 0.329 | −10.674 | 1.00 | 93.76 | O |
| ATOM | 968 | NE2 | GLN | A | 125 | 12.966 | −0.919 | −8.811 | 1.00 | 102.36 | N |
| ATOM | 969 | C | GLN | A | 125 | 14.681 | −4.386 | −11.436 | 1.00 | 89.90 | C |
| ATOM | 970 | O | GLN | A | 125 | 14.502 | −5.366 | −12.182 | 1.00 | 85.04 | O |
| ATOM | 971 | N | LEU | A | 126 | 15.513 | −4.415 | −10.392 | 1.00 | 86.04 | N |
| ATOM | 972 | CA | LEU | A | 126 | 16.399 | −5.552 | −10.122 | 1.00 | 81.17 | C |
| ATOM | 973 | CB | LEU | A | 126 | 17.856 | −5.104 | −10.069 | 1.00 | 76.49 | C |
| ATOM | 974 | CG | LEU | A | 126 | 18.567 | −5.159 | −11.420 | 1.00 | 83.38 | C |
| ATOM | 975 | CD1 | LEU | A | 126 | 20.043 | −5.382 | −11.193 | 1.00 | 87.91 | C |
| ATOM | 976 | CD2 | LEU | A | 126 | 17.989 | −6.247 | −12.330 | 1.00 | 82.09 | C |
| ATOM | 977 | C | LEU | A | 126 | 16.074 | −6.199 | −8.795 | 1.00 | 78.89 | C |
| ATOM | 978 | O | LEU | A | 126 | 16.007 | −5.523 | −7.763 | 1.00 | 80.43 | O |
| ATOM | 979 | N | GLY | A | 127 | 15.922 | −7.518 | −8.810 | 1.00 | 78.57 | N |
| ATOM | 980 | CA | GLY | A | 127 | 15.661 | −8.234 | −7.579 | 1.00 | 80.04 | C |
| ATOM | 981 | C | GLY | A | 127 | 16.864 | −9.043 | −7.153 | 1.00 | 82.63 | C |
| ATOM | 982 | O | GLY | A | 127 | 17.088 | −10.131 | −7.700 | 1.00 | 86.81 | O |
| ATOM | 983 | N | ILE | A | 128 | 17.640 | −8.521 | −6.201 | 1.00 | 76.36 | N |
| ATOM | 984 | CA | ILE | A | 128 | 18.732 | −9.297 | −5.606 | 1.00 | 70.57 | C |
| ATOM | 985 | CB | ILE | A | 128 | 19.927 | −8.404 | −5.254 | 1.00 | 66.04 | C |
| ATOM | 986 | CG1 | ILE | A | 128 | 20.096 | −7.347 | −6.333 | 1.00 | 51.10 | C |
| ATOM | 987 | CD1 | ILE | A | 128 | 19.446 | −6.010 | −5.929 | 1.00 | 72.83 | C |
| ATOM | 988 | CG2 | ILE | A | 128 | 21.202 | −9.223 | −5.047 | 1.00 | 50.44 | C |
| ATOM | 989 | C | ILE | A | 128 | 18.271 | −10.068 | −4.375 | 1.00 | 74.50 | C |
| ATOM | 990 | O | ILE | A | 128 | 17.742 | −9.478 | −3.419 | 1.00 | 76.79 | O |
| ATOM | 991 | N | PRO | A | 129 | 18.511 | −11.379 | −4.399 | 1.00 | 74.83 | N |
| ATOM | 992 | CA | PRO | A | 129 | 18.159 | −12.262 | −3.283 | 1.00 | 78.82 | C |
| ATOM | 993 | CB | PRO | A | 129 | 18.040 | −13.628 | −3.967 | 1.00 | 78.12 | C |
| ATOM | 994 | CG | PRO | A | 129 | 19.069 | −13.563 | −5.071 | 1.00 | 78.35 | C |
| ATOM | 995 | CD | PRO | A | 129 | 19.167 | −12.115 | −5.502 | 1.00 | 72.70 | C |
| ATOM | 996 | C | PRO | A | 129 | 19.247 | −12.276 | −2.197 | 1.00 | 75.59 | C |
| ATOM | 997 | O | PRO | A | 129 | 20.424 | −12.025 | −2.491 | 1.00 | 70.16 | O |
| ATOM | 998 | N | GLU | A | 130 | 18.832 | −12.505 | −0.949 | 1.00 | 73.67 | N |
| ATOM | 999 | CA | GLU | A | 130 | 19.771 | −12.604 | 0.167 | 1.00 | 80.39 | C |
| ATOM | 1000 | CB | GLU | A | 130 | 19.013 | −12.727 | 1.495 | 1.00 | 80.82 | C |
| ATOM | 1001 | CG | GLU | A | 130 | 18.784 | −11.408 | 2.226 | 1.00 | 89.59 | C |
| ATOM | 1002 | CD | GLU | A | 130 | 17.808 | −11.570 | 3.383 | 1.00 | 102.27 | C |
| ATOM | 1003 | OE1 | GLU | A | 130 | 17.496 | −12.722 | 3.759 | 1.00 | 97.11 | O |
| ATOM | 1004 | OE2 | GLU | A | 130 | 17.340 | −10.547 | 3.925 | 1.00 | 105.22 | O |
| ATOM | 1005 | C | GLU | A | 130 | 20.719 | −13.800 | 0.014 | 1.00 | 78.27 | C |
| ATOM | 1006 | O | GLU | A | 130 | 20.275 | −14.951 | −0.107 | 1.00 | 82.45 | O |
| ATOM | 1007 | N | GLN | A | 131 | 22.020 | −13.539 | 0.109 | 1.00 | 69.51 | N |
| ATOM | 1008 | CA | GLN | A | 131 | 22.996 | −14.618 | 0.071 | 1.00 | 65.85 | C |
| ATOM | 1009 | CB | GLN | A | 131 | 23.826 | −14.541 | −1.210 | 1.00 | 65.11 | C |
| ATOM | 1010 | CG | GLN | A | 131 | 22.964 | −14.616 | −2.456 | 1.00 | 69.93 | C |
| ATOM | 1011 | CD | GLN | A | 131 | 23.561 | −13.845 | −3.610 | 1.00 | 83.34 | C |
| ATOM | 1012 | OE1 | GLN | A | 131 | 24.687 | −14.139 | −4.044 | 1.00 | 65.43 | O |
| ATOM | 1013 | NE2 | GLN | A | 131 | 22.813 | −12.859 | −4.116 | 1.00 | 70.99 | N |
| ATOM | 1014 | C | GLN | A | 131 | 23.886 | −14.645 | 1.308 | 1.00 | 65.22 | C |
| ATOM | 1015 | O | GLN | A | 131 | 24.053 | −13.626 | 1.983 | 1.00 | 56.90 | O |
| ATOM | 1016 | N | GLU | A | 132 | 24.376 | −15.837 | 1.648 | 1.00 | 68.52 | N |
| ATOM | 1017 | CA | GLU | A | 132 | 25.386 | −15.998 | 2.697 | 1.00 | 74.33 | C |
| ATOM | 1018 | CB | GLU | A | 132 | 25.138 | −17.274 | 3.517 | 1.00 | 75.45 | C |
| ATOM | 1019 | CG | GLU | A | 132 | 24.174 | −17.114 | 4.694 | 1.00 | 85.36 | C |
| ATOM | 1020 | CD | GLU | A | 132 | 24.717 | −17.681 | 6.007 | 1.00 | 87.01 | C |
| ATOM | 1021 | OE1 | GLU | A | 132 | 24.080 | −18.584 | 6.619 | 1.00 | 71.41 | O |
| ATOM | 1022 | OE2 | GLU | A | 132 | 25.782 | −17.197 | 6.445 | 1.00 | 80.55 | O |
| ATOM | 1023 | C | GLU | A | 132 | 26.806 | −16.001 | 2.111 | 1.00 | 74.23 | C |
| ATOM | 1024 | O | GLU | A | 132 | 27.153 | −16.840 | 1.265 | 1.00 | 79.48 | O |
| ATOM | 1025 | N | TYR | A | 133 | 27.615 | −15.038 | 2.542 | 1.00 | 64.41 | N |
| ATOM | 1026 | CA | TYR | A | 133 | 28.874 | −14.751 | 1.871 | 1.00 | 56.63 | C |
| ATOM | 1027 | CB | TYR | A | 133 | 29.130 | −13.245 | 1.847 | 1.00 | 50.92 | C |
| ATOM | 1028 | CG | TYR | A | 133 | 28.089 | −12.478 | 1.072 | 1.00 | 45.22 | C |
| ATOM | 1029 | CD1 | TYR | A | 133 | 27.095 | −11.770 | 1.732 | 1.00 | 65.45 | C |
| ATOM | 1030 | CE1 | TYR | A | 133 | 26.147 | −11.044 | 1.030 | 1.00 | 52.85 | C |
| ATOM | 1031 | CZ | TYR | A | 133 | 26.198 | −11.028 | −0.347 | 1.00 | 30.15 | C |
| ATOM | 1032 | OH | TYR | A | 133 | 25.249 | −10.334 | −1.044 | 1.00 | 55.61 | O |
| ATOM | 1033 | CE2 | TYR | A | 133 | 27.183 | −11.703 | −1.024 | 1.00 | 37.77 | C |
| ATOM | 1034 | CD2 | TYR | A | 133 | 28.117 | −12.425 | −0.318 | 1.00 | 49.45 | C |
| ATOM | 1035 | C | TYR | A | 133 | 30.023 | −15.474 | 2.554 | 1.00 | 51.83 | C |
| ATOM | 1036 | O | TYR | A | 133 | 29.944 | −15.781 | 3.734 | 1.00 | 48.99 | O |
| ATOM | 1037 | N | SER | A | 134 | 31.097 | −15.721 | 1.815 | 1.00 | 53.70 | N |
| ATOM | 1038 | CA | SER | A | 134 | 32.211 | −16.515 | 2.322 | 1.00 | 55.41 | C |
| ATOM | 1039 | CB | SER | A | 134 | 33.138 | −16.935 | 1.178 | 1.00 | 55.91 | C |
| ATOM | 1040 | OG | SER | A | 134 | 32.534 | −17.935 | 0.380 | 1.00 | 46.54 | O |
| ATOM | 1041 | C | SER | A | 134 | 33.026 | −15.729 | 3.329 | 1.00 | 54.13 | C |
| ATOM | 1042 | O | SER | A | 134 | 33.592 | −16.310 | 4.264 | 1.00 | 44.28 | O |
| ATOM | 1043 | N | CYS | A | 135 | 33.184 | −14.436 | 3.043 | 1.00 | 54.42 | N |
| ATOM | 1044 | CA | CYS | A | 135 | 33.972 | −13.531 | 3.880 | 1.00 | 53.39 | C |
| ATOM | 1045 | CB | CYS | A | 135 | 35.343 | −13.243 | 3.273 | 1.00 | 49.65 | C |
| ATOM | 1046 | SG | CYS | A | 135 | 36.465 | −14.627 | 3.537 | 1.00 | 75.39 | S |
| ATOM | 1047 | C | CYS | A | 135 | 33.247 | −12.228 | 4.098 | 1.00 | 44.71 | C |
| ATOM | 1048 | O | CYS | A | 135 | 32.747 | −11.628 | 3.151 | 1.00 | 44.42 | O |
| ATOM | 1049 | N | VAL | A | 136 | 33.215 | −11.786 | 5.349 | 1.00 | 39.33 | N |
| ATOM | 1050 | CA | VAL | A | 136 | 32.716 | −10.462 | 5.666 | 1.00 | 32.29 | C |
| ATOM | 1051 | CB | VAL | A | 136 | 31.458 | −10.512 | 6.558 | 1.00 | 31.88 | C |
| ATOM | 1052 | CG1 | VAL | A | 136 | 30.936 | −9.116 | 6.813 | 1.00 | 14.33 | C |
| ATOM | 1053 | CG2 | VAL | A | 136 | 30.357 | −11.406 | 5.942 | 1.00 | 17.10 | C |
| ATOM | 1054 | C | VAL | A | 136 | 33.824 | −9.750 | 6.411 | 1.00 | 38.07 | C |
| ATOM | 1055 | O | VAL | A | 136 | 34.125 | −10.084 | 7.549 | 1.00 | 45.62 | O |
| ATOM | 1056 | N | VAL | A | 137 | 34.447 | −8.783 | 5.751 | 1.00 | 37.26 | N |
| ATOM | 1057 | CA | VAL | A | 137 | 35.499 | −7.999 | 6.368 | 1.00 | 43.95 | C |
| ATOM | 1058 | CB | VAL | A | 137 | 36.572 | −7.644 | 5.336 | 1.00 | 45.57 | C |
| ATOM | 1059 | CG1 | VAL | A | 137 | 37.765 | −6.974 | 6.011 | 1.00 | 45.81 | C |
| ATOM | 1060 | CG2 | VAL | A | 137 | 36.991 | −8.897 | 4.585 | 1.00 | 52.36 | C |
| ATOM | 1061 | C | VAL | A | 137 | 34.909 | −6.722 | 6.951 | 1.00 | 48.64 | C |
| ATOM | 1062 | O | VAL | A | 137 | 34.186 | −5.992 | 6.270 | 1.00 | 56.48 | O |
| ATOM | 1063 | N | LYS | A | 138 | 35.182 | −6.459 | 8.222 | 1.00 | 47.79 | N |
| ATOM | 1064 | CA | LYS | A | 138 | 34.758 | −5.190 | 8.806 | 1.00 | 53.20 | C |
| ATOM | 1065 | CB | LYS | A | 138 | 34.004 | −5.426 | 10.128 | 1.00 | 50.85 | C |
| ATOM | 1066 | CG | LYS | A | 138 | 33.516 | −4.148 | 10.819 | 1.00 | 70.17 | C |
| ATOM | 1067 | CD | LYS | A | 138 | 34.210 | −3.910 | 12.188 | 1.00 | 90.42 | C |
| ATOM | 1068 | CE | LYS | A | 138 | 33.598 | −2.738 | 13.005 | 1.00 | 88.45 | C |
| ATOM | 1069 | NZ | LYS | A | 138 | 32.716 | −3.114 | 14.174 | 1.00 | 47.89 | N |
| ATOM | 1070 | C | LYS | A | 138 | 35.987 | −4.301 | 9.008 | 1.00 | 53.76 | C |
| ATOM | 1071 | O | LYS | A | 138 | 37.020 | −4.764 | 9.502 | 1.00 | 60.17 | O |
| ATOM | 1072 | N | MET | A | 139 | 35.918 | −3.051 | 8.563 | 1.00 | 52.99 | N |
| ATOM | 1073 | CA | MET | A | 139 | 37.044 | −2.136 | 8.733 | 1.00 | 43.81 | C |
| ATOM | 1074 | CB | MET | A | 139 | 38.076 | −2.327 | 7.616 | 1.00 | 47.36 | C |
| ATOM | 1075 | CG | MET | A | 139 | 37.531 | −2.938 | 6.346 | 1.00 | 43.72 | C |
| ATOM | 1076 | SD | MET | A | 139 | 38.370 | −2.210 | 4.957 | 1.00 | 73.58 | S |
| ATOM | 1077 | CE | MET | A | 139 | 39.975 | −2.875 | 5.230 | 1.00 | 39.82 | C |
| ATOM | 1078 | C | MET | A | 139 | 36.583 | −0.685 | 8.790 | 1.00 | 40.43 | C |
| ATOM | 1079 | O | MET | A | 139 | 35.464 | −0.363 | 8.374 | 1.00 | 34.45 | O |
| ATOM | 1080 | N | PRO | A | 140 | 37.420 | 0.179 | 9.356 | 1.00 | 38.95 | N |
| ATOM | 1081 | CA | PRO | A | 140 | 37.139 | 1.619 | 9.396 | 1.00 | 43.44 | C |
| ATOM | 1082 | CB | PRO | A | 140 | 38.419 | 2.217 | 9.976 | 1.00 | 41.89 | C |
| ATOM | 1083 | CG | PRO | A | 140 | 39.063 | 1.112 | 10.722 | 1.00 | 37.32 | C |
| ATOM | 1084 | CD | PRO | A | 140 | 38.662 | −0.172 | 10.062 | 1.00 | 42.37 | C |
| ATOM | 1085 | C | PRO | A | 140 | 36.931 | 2.172 | 7.999 | 1.00 | 48.88 | C |
| ATOM | 1086 | O | PRO | A | 140 | 37.681 | 1.826 | 7.072 | 1.00 | 41.66 | O |
| ATOM | 1087 | N | SER | A | 141 | 35.923 | 3.033 | 7.863 | 1.00 | 48.06 | N |
| ATOM | 1088 | CA | SER | A | 141 | 35.558 | 3.594 | 6.567 | 1.00 | 38.28 | C |
| ATOM | 1089 | CB | SER | A | 141 | 34.246 | 4.369 | 6.693 | 1.00 | 34.65 | C |
| ATOM | 1090 | OG | SER | A | 141 | 34.344 | 5.396 | 7.682 | 1.00 | 39.09 | O |
| ATOM | 1091 | C | SER | A | 141 | 36.668 | 4.485 | 5.996 | 1.00 | 33.35 | C |
| ATOM | 1092 | O | SER | A | 141 | 36.857 | 4.544 | 4.790 | 1.00 | 38.05 | O |
| ATOM | 1093 | N | GLY | A | 142 | 37.368 | 5.210 | 6.862 | 1.00 | 25.94 | N |
| ATOM | 1094 | CA | GLY | A | 142 | 38.506 | 6.010 | 6.451 | 1.00 | 23.29 | C |
| ATOM | 1095 | C | GLY | A | 142 | 39.705 | 5.190 | 6.009 | 1.00 | 29.00 | C |
| ATOM | 1096 | O | GLY | A | 142 | 40.536 | 5.681 | 5.261 | 1.00 | 31.59 | O |
| ATOM | 1097 | N | GLU | A | 143 | 39.832 | 3.964 | 6.513 | 1.00 | 39.78 | N |
| ATOM | 1098 | CA | GLU | A | 143 | 40.932 | 3.070 | 6.144 | 1.00 | 34.94 | C |
| ATOM | 1099 | CB | GLU | A | 143 | 40.962 | 1.855 | 7.080 | 1.00 | 40.67 | C |
| ATOM | 1100 | CG | GLU | A | 143 | 42.232 | 1.009 | 7.021 | 1.00 | 62.26 | C |
| ATOM | 1101 | CD | GLU | A | 143 | 43.402 | 1.623 | 7.786 | 1.00 | 90.23 | C |
| ATOM | 1102 | OE1 | GLU | A | 143 | 43.734 | 1.140 | 8.902 | 1.00 | 69.87 | O |
| ATOM | 1103 | OE2 | GLU | A | 143 | 44.014 | 2.582 | 7.253 | 1.00 | 97.38 | O |
| ATOM | 1104 | C | GLU | A | 143 | 40.725 | 2.573 | 4.719 | 1.00 | 34.31 | C |
| ATOM | 1105 | O | GLU | A | 143 | 41.671 | 2.491 | 3.931 | 1.00 | 31.54 | O |
| ATOM | 1106 | N | PHE | A | 144 | 39.495 | 2.157 | 4.428 | 1.00 | 33.21 | N |
| ATOM | 1107 | CA | PHE | A | 144 | 39.173 | 1.614 | 3.122 | 1.00 | 17.60 | C |
| ATOM | 1108 | CB | PHE | A | 144 | 37.788 | 1.004 | 3.122 | 1.00 | 9.51 | C |
| ATOM | 1109 | CG | PHE | A | 144 | 37.485 | 0.283 | 1.872 | 1.00 | 18.10 | C |
| ATOM | 1110 | CD1 | PHE | A | 144 | 38.333 | −0.718 | 1.441 | 1.00 | 25.44 | C |
| ATOM | 1111 | CE1 | PHE | A | 144 | 38.095 | −1.387 | 0.256 | 1.00 | 21.29 | C |
| ATOM | 1112 | CZ | PHE | A | 144 | 37.039 | −0.988 | −0.549 | 1.00 | 46.82 | C |
| ATOM | 1113 | CE2 | PHE | A | 144 | 36.168 | 0.032 | −0.126 | 1.00 | 27.23 | C |
| ATOM | 1114 | CD2 | PHE | A | 144 | 36.403 | 0.657 | 1.073 | 1.00 | 6.51 | C |
| ATOM | 1115 | C | PHE | A | 144 | 39.256 | 2.710 | 2.070 | 1.00 | 20.30 | C |
| ATOM | 1116 | O | PHE | A | 144 | 39.576 | 2.430 | 0.928 | 1.00 | 31.67 | O |
| ATOM | 1117 | N | ALA | A | 145 | 38.985 | 3.957 | 2.435 | 1.00 | 21.53 | N |
| ATOM | 1118 | CA | ALA | A | 145 | 39.009 | 5.030 | 1.452 | 1.00 | 21.40 | C |
| ATOM | 1119 | CB | ALA | A | 145 | 38.475 | 6.321 | 2.049 | 1.00 | 24.12 | C |
| ATOM | 1120 | C | ALA | A | 145 | 40.439 | 5.225 | 1.006 | 1.00 | 25.44 | C |
| ATOM | 1121 | O | ALA | A | 145 | 40.701 | 5.611 | −0.118 | 1.00 | 32.97 | O |
| ATOM | 1122 | N | ARG | A | 146 | 41.365 | 5.065 | 1.934 | 1.00 | 27.03 | N |
| ATOM | 1123 | CA | ARG | A | 146 | 42.748 | 5.419 | 1.657 | 1.00 | 25.94 | C |
| ATOM | 1124 | CB | ARG | A | 146 | 43.536 | 5.667 | 2.957 | 1.00 | 21.72 | C |
| ATOM | 1125 | CG | ARG | A | 146 | 44.969 | 6.108 | 2.773 | 1.00 | 14.50 | C |
| ATOM | 1126 | CD | ARG | A | 146 | 45.147 | 7.625 | 2.872 | 1.00 | 48.85 | C |
| ATOM | 1127 | NE | ARG | A | 146 | 46.383 | 8.047 | 3.538 | 1.00 | 62.08 | N |
| ATOM | 1128 | CZ | ARG | A | 146 | 47.581 | 8.104 | 2.947 | 1.00 | 69.45 | C |
| ATOM | 1129 | NH1 | ARG | A | 146 | 47.715 | 7.694 | 1.687 | 1.00 | 54.13 | N |
| ATOM | 1130 | NH2 | ARG | A | 146 | 48.646 | 8.548 | 3.617 | 1.00 | 76.45 | N |
| ATOM | 1131 | C | ARG | A | 146 | 43.327 | 4.269 | 0.842 | 1.00 | 30.34 | C |
| ATOM | 1132 | O | ARG | A | 146 | 44.050 | 4.491 | −0.126 | 1.00 | 44.72 | O |
| ATOM | 1133 | N | ILE | A | 147 | 42.950 | 3.041 | 1.164 | 1.00 | 14.95 | N |
| ATOM | 1134 | CA | ILE | A | 147 | 43.553 | 1.928 | 0.462 | 1.00 | 10.88 | C |
| ATOM | 1135 | CB | ILE | A | 147 | 43.046 | 0.633 | 1.043 | 1.00 | 12.22 | C |
| ATOM | 1136 | CG1 | ILE | A | 147 | 43.719 | 0.338 | 2.381 | 1.00 | 21.72 | C |
| ATOM | 1137 | CD1 | ILE | A | 147 | 42.977 | −0.700 | 3.239 | 1.00 | 3.19 | C |
| ATOM | 1138 | CG2 | ILE | A | 147 | 43.278 | −0.507 | 0.102 | 1.00 | 20.91 | C |
| ATOM | 1139 | C | ILE | A | 147 | 43.172 | 2.021 | −1.003 | 1.00 | 18.70 | C |
| ATOM | 1140 | O | ILE | A | 147 | 44.009 | 1.890 | −1.896 | 1.00 | 20.16 | O |
| ATOM | 1141 | N | CYS | A | 148 | 41.898 | 2.307 | −1.244 | 1.00 | 25.75 | N |
| ATOM | 1142 | CA | CYS | A | 148 | 41.396 | 2.399 | −2.602 | 1.00 | 21.58 | C |
| ATOM | 1143 | CB | CYS | A | 148 | 39.874 | 2.575 | −2.596 | 1.00 | 25.84 | C |
| ATOM | 1144 | SG | CYS | A | 148 | 38.938 | 1.039 | −2.338 | 1.00 | 38.63 | S |
| ATOM | 1145 | C | CYS | A | 148 | 42.087 | 3.577 | −3.281 | 1.00 | 26.83 | C |
| ATOM | 1146 | O | CYS | A | 148 | 42.286 | 3.557 | −4.496 | 1.00 | 30.23 | O |
| ATOM | 1147 | N | ARG | A | 149 | 42.433 | 4.614 | −2.519 | 1.00 | 27.92 | N |
| ATOM | 1148 | CA | ARG | A | 149 | 42.898 | 5.839 | −3.155 | 1.00 | 25.18 | C |
| ATOM | 1149 | CB | ARG | A | 149 | 42.781 | 7.060 | −2.255 | 1.00 | 28.35 | C |
| ATOM | 1150 | CG | ARG | A | 149 | 42.599 | 8.354 | −3.038 | 1.00 | 36.70 | C |
| ATOM | 1151 | CD | ARG | A | 149 | 43.452 | 9.495 | −2.512 | 1.00 | 30.53 | C |
| ATOM | 1152 | NE | ARG | A | 149 | 43.594 | 9.409 | −1.062 | 1.00 | 72.27 | N |
| ATOM | 1153 | CZ | ARG | A | 149 | 43.295 | 10.398 | −0.231 | 1.00 | 86.77 | C |
| ATOM | 1154 | NH1 | ARG | A | 149 | 42.884 | 11.571 | −0.701 | 1.00 | 76.33 | N |
| ATOM | 1155 | NH2 | ARG | A | 149 | 43.451 | 10.235 | 1.074 | 1.00 | 99.05 | N |
| ATOM | 1156 | C | ARG | A | 149 | 44.330 | 5.646 | −3.503 | 1.00 | 32.36 | C |
| ATOM | 1157 | O | ARG | A | 149 | 44.821 | 6.159 | −4.505 | 1.00 | 46.67 | O |
| ATOM | 1158 | N | ASP | A | 150 | 45.020 | 4.884 | −2.685 | 1.00 | 32.78 | N |
| ATOM | 1159 | CA | ASP | A | 150 | 46.434 | 4.768 | −2.935 | 1.00 | 36.27 | C |
| ATOM | 1160 | CB | ASP | A | 150 | 47.168 | 4.441 | −1.636 | 1.00 | 41.29 | C |
| ATOM | 1161 | CG | ASP | A | 150 | 47.037 | 5.560 | −0.601 | 1.00 | 52.19 | C |
| ATOM | 1162 | OD1 | ASP | A | 150 | 46.723 | 6.705 | −0.989 | 1.00 | 57.17 | O |
| ATOM | 1163 | OD2 | ASP | A | 150 | 47.190 | 5.373 | 0.631 | 1.00 | 63.67 | O |
| ATOM | 1164 | C | ASP | A | 150 | 46.698 | 3.741 | −4.042 | 1.00 | 31.43 | C |
| ATOM | 1165 | O | ASP | A | 150 | 47.636 | 3.879 | −4.835 | 1.00 | 26.77 | O |
| ATOM | 1166 | N | LEU | A | 151 | 45.851 | 2.727 | −4.137 | 1.00 | 21.77 | N |
| ATOM | 1167 | CA | LEU | A | 151 | 46.067 | 1.730 | −5.168 | 1.00 | 19.08 | C |
| ATOM | 1168 | CB | LEU | A | 151 | 45.338 | 0.446 | −4.826 | 1.00 | 15.67 | C |
| ATOM | 1169 | CG | LEU | A | 151 | 45.940 | −0.282 | −3.619 | 1.00 | 17.15 | C |
| ATOM | 1170 | CD1 | LEU | A | 151 | 45.211 | −1.615 | −3.393 | 1.00 | 25.26 | C |
| ATOM | 1171 | CD2 | LEU | A | 151 | 47.436 | −0.478 | −3.789 | 1.00 | 7.26 | C |
| ATOM | 1172 | C | LEU | A | 151 | 45.634 | 2.227 | −6.529 | 1.00 | 26.52 | C |
| ATOM | 1173 | O | LEU | A | 151 | 46.064 | 1.693 | −7.538 | 1.00 | 29.20 | O |
| ATOM | 1174 | N | SER | A | 152 | 44.805 | 3.271 | −6.550 | 1.00 | 37.48 | N |
| ATOM | 1175 | CA | SER | A | 152 | 44.391 | 3.943 | −7.784 | 1.00 | 34.56 | C |
| ATOM | 1176 | CB | SER | A | 152 | 43.439 | 5.096 | −7.480 | 1.00 | 28.23 | C |
| ATOM | 1177 | OG | SER | A | 152 | 42.111 | 4.616 | −7.445 | 1.00 | 56.03 | O |
| ATOM | 1178 | C | SER | A | 152 | 45.579 | 4.511 | −8.523 | 1.00 | 34.52 | C |
| ATOM | 1179 | O | SER | A | 152 | 45.535 | 4.652 | −9.744 | 1.00 | 48.31 | O |
| ATOM | 1180 | N | HIS | A | 153 | 46.598 | 4.915 | −7.771 | 1.00 | 30.14 | N |
| ATOM | 1181 | CA | HIS | A | 153 | 47.765 | 5.558 | −8.351 | 1.00 | 30.39 | C |
| ATOM | 1182 | CB | HIS | A | 153 | 48.529 | 6.372 | −7.309 | 1.00 | 33.57 | C |
| ATOM | 1183 | CG | HIS | A | 153 | 47.837 | 7.641 | −6.896 | 1.00 | 60.36 | C |
| ATOM | 1184 | ND1 | HIS | A | 153 | 46.704 | 8.121 | −7.529 | 1.00 | 51.42 | N |
| ATOM | 1185 | CE1 | HIS | A | 153 | 46.273 | 9.201 | −6.898 | 1.00 | 60.55 | C |
| ATOM | 1186 | NE2 | HIS | A | 153 | 47.123 | 9.478 | −5.923 | 1.00 | 70.44 | N |
| ATOM | 1187 | CD2 | HIS | A | 153 | 48.106 | 8.515 | −5.893 | 1.00 | 57.62 | C |
| ATOM | 1188 | C | HIS | A | 153 | 48.662 | 4.490 | −8.919 | 1.00 | 30.20 | C |
| ATOM | 1189 | O | HIS | A | 153 | 49.514 | 4.779 | −9.753 | 1.00 | 37.51 | O |
| ATOM | 1190 | N | ILE | A | 154 | 48.438 | 3.250 | −8.498 | 1.00 | 27.82 | N |
| ATOM | 1191 | CA | ILE | A | 154 | 49.272 | 2.143 | −8.956 | 1.00 | 28.85 | C |
| ATOM | 1192 | CB | ILE | A | 154 | 49.360 | 1.054 | −7.891 | 1.00 | 34.86 | C |
| ATOM | 1193 | CG1 | ILE | A | 154 | 49.911 | 1.598 | −6.585 | 1.00 | 9.51 | C |
| ATOM | 1194 | CD1 | ILE | A | 154 | 51.295 | 2.010 | −6.698 | 1.00 | 13.75 | C |
| ATOM | 1195 | CG2 | ILE | A | 154 | 50.223 | −0.045 | −8.368 | 1.00 | 18.44 | C |
| ATOM | 1196 | C | ILE | A | 154 | 48.656 | 1.522 | −10.208 | 1.00 | 33.71 | C |
| ATOM | 1197 | O | ILE | A | 154 | 49.231 | 1.581 | −11.280 | 1.00 | 45.31 | O |
| ATOM | 1198 | N | GLY | A | 155 | 47.499 | 0.888 | −10.074 | 1.00 | 35.21 | N |
| ATOM | 1199 | CA | GLY | A | 155 | 46.876 | 0.209 | −11.198 | 1.00 | 30.85 | C |
| ATOM | 1200 | C | GLY | A | 155 | 45.389 | 0.504 | −11.306 | 1.00 | 32.90 | C |
| ATOM | 1201 | O | GLY | A | 155 | 44.869 | 1.339 | −10.585 | 1.00 | 33.47 | O |
| ATOM | 1202 | N | ASP | A | 156 | 44.713 | −0.123 | −12.262 | 1.00 | 37.33 | N |
| ATOM | 1203 | CA | ASP | A | 156 | 43.298 | 0.147 | −12.512 | 1.00 | 31.47 | C |
| ATOM | 1204 | CB | ASP | A | 156 | 42.980 | 0.084 | −14.011 | 1.00 | 25.78 | C |
| ATOM | 1205 | CG | ASP | A | 156 | 43.151 | 1.439 | −14.695 | 1.00 | 49.70 | C |
| ATOM | 1206 | OD1 | ASP | A | 156 | 43.796 | 2.316 | −14.063 | 1.00 | 47.56 | O |
| ATOM | 1207 | OD2 | ASP | A | 156 | 42.665 | 1.729 | −15.826 | 1.00 | 38.74 | O |
| ATOM | 1208 | C | ASP | A | 156 | 42.402 | −0.803 | −11.720 | 1.00 | 20.25 | C |
| ATOM | 1209 | O | ASP | A | 156 | 41.262 | −0.487 | −11.435 | 1.00 | 31.72 | O |
| ATOM | 1210 | N | ALA | A | 157 | 42.930 | −1.960 | −11.361 | 1.00 | 10.69 | N |
| ATOM | 1211 | CA | ALA | A | 157 | 42.143 | −2.994 | −10.728 | 1.00 | 21.77 | C |
| ATOM | 1212 | CB | ALA | A | 157 | 41.919 | −4.166 | −11.691 | 1.00 | 17.75 | C |
| ATOM | 1213 | C | ALA | A | 157 | 42.858 | −3.475 | −9.463 | 1.00 | 33.18 | C |
| ATOM | 1214 | O | ALA | A | 157 | 44.088 | −3.418 | −9.361 | 1.00 | 40.10 | O |
| ATOM | 1215 | N | VAL | A | 158 | 42.077 | −3.953 | −8.501 | 1.00 | 28.23 | N |
| ATOM | 1216 | CA | VAL | A | 158 | 42.618 | −4.370 | −7.225 | 1.00 | 23.49 | C |
| ATOM | 1217 | CB | VAL | A | 158 | 42.067 | −3.511 | −6.058 | 1.00 | 19.38 | C |
| ATOM | 1218 | CG1 | VAL | A | 158 | 40.595 | −3.246 | −6.223 | 1.00 | 31.27 | C |
| ATOM | 1219 | CG2 | VAL | A | 158 | 42.284 | −4.220 | −4.727 | 1.00 | 7.72 | C |
| ATOM | 1220 | C | VAL | A | 158 | 42.292 | −5.840 | −7.006 | 1.00 | 29.53 | C |
| ATOM | 1221 | O | VAL | A | 158 | 41.140 | −6.262 | −7.156 | 1.00 | 28.38 | O |
| ATOM | 1222 | N | VAL | A | 159 | 43.320 | −6.611 | −6.646 | 1.00 | 41.12 | N |
| ATOM | 1223 | CA | VAL | A | 159 | 43.180 | −8.021 | −6.269 | 1.00 | 39.57 | C |
| ATOM | 1224 | CB | VAL | A | 159 | 44.403 | −8.830 | −6.715 | 1.00 | 36.37 | C |
| ATOM | 1225 | CG1 | VAL | A | 159 | 44.170 | −10.323 | −6.495 | 1.00 | 15.62 | C |
| ATOM | 1226 | CG2 | VAL | A | 159 | 44.761 | −8.485 | −8.168 | 1.00 | 29.68 | C |
| ATOM | 1227 | C | VAL | A | 159 | 43.007 | −8.201 | −4.761 | 1.00 | 42.20 | C |
| ATOM | 1228 | O | VAL | A | 159 | 43.944 | −7.994 | −3.991 | 1.00 | 43.58 | O |
| ATOM | 1229 | N | ILE | A | 160 | 41.787 | −8.514 | −4.340 | 1.00 | 33.02 | N |
| ATOM | 1230 | CA | ILE | A | 160 | 41.508 | −8.695 | −2.928 | 1.00 | 30.11 | C |
| ATOM | 1231 | CB | ILE | A | 160 | 40.150 | −8.105 | −2.598 | 1.00 | 22.47 | C |
| ATOM | 1232 | CG1 | ILE | A | 160 | 40.107 | −6.636 | −3.001 | 1.00 | 18.48 | C |
| ATOM | 1233 | CD1 | ILE | A | 160 | 38.771 | −5.998 | −2.697 | 1.00 | 10.36 | C |
| ATOM | 1234 | CG2 | ILE | A | 160 | 39.836 | −8.273 | −1.124 | 1.00 | 18.55 | C |
| ATOM | 1235 | C | ILE | A | 160 | 41.530 | −10.182 | −2.553 | 1.00 | 44.46 | C |
| ATOM | 1236 | O | ILE | A | 160 | 40.703 | −10.971 | −3.030 | 1.00 | 48.56 | O |
| ATOM | 1237 | N | SER | A | 161 | 42.481 | −10.579 | −1.708 | 1.00 | 48.92 | N |
| ATOM | 1238 | CA | SER | A | 161 | 42.524 | −11.964 | −1.243 | 1.00 | 48.64 | C |
| ATOM | 1239 | CB | SER | A | 161 | 43.881 | −12.585 | −1.553 | 1.00 | 60.11 | C |
| ATOM | 1240 | OG | SER | A | 161 | 44.444 | −11.997 | −2.716 | 1.00 | 76.76 | O |
| ATOM | 1241 | C | SER | A | 161 | 42.243 | −12.048 | 0.247 | 1.00 | 45.29 | C |
| ATOM | 1242 | O | SER | A | 161 | 42.623 | −11.170 | 1.012 | 1.00 | 49.84 | O |
| ATOM | 1243 | N | CYS | A | 162 | 41.533 | −13.087 | 0.644 | 1.00 | 34.00 | N |
| ATOM | 1244 | CA | CYS | A | 162 | 41.010 | −13.163 | 1.985 | 1.00 | 41.29 | C |
| ATOM | 1245 | CB | CYS | A | 162 | 39.501 | −13.018 | 1.923 | 1.00 | 29.40 | C |
| ATOM | 1246 | SG | CYS | A | 162 | 38.798 | −12.195 | 3.370 | 1.00 | 89.53 | S |
| ATOM | 1247 | C | CYS | A | 162 | 41.395 | −14.520 | 2.574 | 1.00 | 50.61 | C |
| ATOM | 1248 | O | CYS | A | 162 | 41.432 | −15.504 | 1.849 | 1.00 | 53.07 | O |
| ATOM | 1249 | N | ALA | A | 163 | 41.815 | −14.556 | 3.840 | 1.00 | 58.73 | N |
| ATOM | 1250 | CA | ALA | A | 163 | 42.202 | −15.809 | 4.496 | 1.00 | 65.94 | C |
| ATOM | 1251 | CB | ALA | A | 163 | 43.729 | −16.009 | 4.481 | 1.00 | 61.28 | C |
| ATOM | 1252 | C | ALA | A | 163 | 41.663 | −15.870 | 5.924 | 1.00 | 69.87 | C |
| ATOM | 1253 | O | ALA | A | 163 | 40.546 | −15.388 | 6.194 | 1.00 | 69.92 | O |
| ATOM | 1254 | N | LYS | A | 164 | 42.456 | −16.456 | 6.834 | 1.00 | 75.82 | N |
| ATOM | 1255 | CA | LYS | A | 164 | 42.136 | −16.421 | 8.276 | 1.00 | 81.70 | C |
| ATOM | 1256 | CB | LYS | A | 164 | 42.127 | −17.846 | 8.878 | 1.00 | 81.72 | C |
| ATOM | 1257 | CG | LYS | A | 164 | 42.208 | −17.888 | 10.402 | 1.00 | 69.68 | C |
| ATOM | 1258 | CD | LYS | A | 164 | 40.935 | −18.485 | 11.001 | 1.00 | 86.51 | C |
| ATOM | 1259 | CE | LYS | A | 164 | 40.495 | −17.661 | 12.197 | 1.00 | 89.07 | C |
| ATOM | 1260 | NZ | LYS | A | 164 | 39.023 | −17.435 | 12.178 | 1.00 | 70.52 | N |
| ATOM | 1261 | C | LYS | A | 164 | 43.087 | −15.506 | 9.075 | 1.00 | 82.06 | C |
| ATOM | 1262 | O | LYS | A | 164 | 42.718 | −14.959 | 10.132 | 1.00 | 73.06 | O |
| ATOM | 1263 | N | ASP | A | 165 | 44.298 | −15.347 | 8.532 | 1.00 | 77.01 | N |
| ATOM | 1264 | CA | ASP | A | 165 | 45.275 | −14.343 | 8.963 | 1.00 | 72.33 | C |
| ATOM | 1265 | CB | ASP | A | 165 | 46.574 | −14.501 | 8.153 | 1.00 | 70.65 | C |
| ATOM | 1266 | CG | ASP | A | 165 | 46.921 | −15.963 | 7.897 | 1.00 | 72.63 | C |
| ATOM | 1267 | OD1 | ASP | A | 165 | 46.088 | −16.671 | 7.270 | 1.00 | 64.79 | O |
| ATOM | 1268 | OD2 | ASP | A | 165 | 47.951 | −16.517 | 8.362 | 1.00 | 71.13 | O |
| ATOM | 1269 | C | ASP | A | 165 | 44.750 | −12.912 | 8.822 | 1.00 | 65.46 | C |
| ATOM | 1270 | O | ASP | A | 165 | 44.503 | −12.227 | 9.829 | 1.00 | 64.83 | O |
| ATOM | 1271 | N | GLY | A | 166 | 44.614 | −12.459 | 7.570 | 1.00 | 55.73 | N |
| ATOM | 1272 | CA | GLY | A | 166 | 43.727 | −11.313 | 7.293 | 1.00 | 44.69 | C |
| ATOM | 1273 | C | GLY | A | 166 | 43.605 | −11.119 | 5.790 | 1.00 | 44.20 | C |
| ATOM | 1274 | O | GLY | A | 166 | 43.693 | −12.087 | 5.018 | 1.00 | 33.93 | O |
| ATOM | 1275 | N | VAL | A | 167 | 43.358 | −9.877 | 5.382 | 1.00 | 32.81 | N |
| ATOM | 1276 | CA | VAL | A | 167 | 43.013 | −9.582 | 4.006 | 1.00 | 24.18 | C |
| ATOM | 1277 | CB | VAL | A | 167 | 41.728 | −8.732 | 3.901 | 1.00 | 23.59 | C |
| ATOM | 1278 | CG1 | VAL | A | 167 | 41.669 | −7.693 | 4.982 | 1.00 | 30.98 | C |
| ATOM | 1279 | CG2 | VAL | A | 167 | 41.653 | −8.063 | 2.536 | 1.00 | 41.26 | C |
| ATOM | 1280 | C | VAL | A | 167 | 44.169 | −8.860 | 3.367 | 1.00 | 14.85 | C |
| ATOM | 1281 | O | VAL | A | 167 | 44.725 | −7.967 | 3.973 | 1.00 | 15.49 | O |
| ATOM | 1282 | N | LYS | A | 168 | 44.491 | −9.190 | 2.122 | 1.00 | 21.54 | N |
| ATOM | 1283 | CA | LYS | A | 168 | 45.529 | −8.460 | 1.375 | 1.00 | 25.14 | C |
| ATOM | 1284 | CB | LYS | A | 168 | 46.734 | −9.365 | 1.091 | 1.00 | 23.76 | C |
| ATOM | 1285 | CG | LYS | A | 168 | 47.731 | −8.827 | 0.061 | 1.00 | 26.85 | C |
| ATOM | 1286 | CD | LYS | A | 168 | 48.946 | −9.747 | −0.089 | 1.00 | 43.57 | C |
| ATOM | 1287 | CE | LYS | A | 168 | 48.832 | −10.666 | −1.301 | 1.00 | 57.61 | C |
| ATOM | 1288 | NZ | LYS | A | 168 | 49.223 | −12.088 | −0.970 | 1.00 | 55.21 | N |
| ATOM | 1289 | C | LYS | A | 168 | 45.012 | −7.846 | 0.067 | 1.00 | 28.58 | C |
| ATOM | 1290 | O | LYS | A | 168 | 44.332 | −8.499 | −0.734 | 1.00 | 31.58 | O |
| ATOM | 1291 | N | PHE | A | 169 | 45.308 | −6.569 | −0.122 | 1.00 | 17.11 | N |
| ATOM | 1292 | CA | PHE | A | 169 | 44.926 | −5.873 | −1.334 | 1.00 | 11.37 | C |
| ATOM | 1293 | CB | PHE | A | 169 | 44.389 | −4.506 | −0.964 | 1.00 | 11.65 | C |
| ATOM | 1294 | CG | PHE | A | 169 | 43.181 | −4.550 | −0.081 | 1.00 | 16.62 | C |
| ATOM | 1295 | CD1 | PHE | A | 169 | 43.299 | −4.476 | 1.308 | 1.00 | 17.61 | C |
| ATOM | 1296 | CE1 | PHE | A | 169 | 42.179 | −4.450 | 2.112 | 1.00 | 17.71 | C |
| ATOM | 1297 | CZ | PHE | A | 169 | 40.920 | −4.470 | 1.529 | 1.00 | 16.97 | C |
| ATOM | 1298 | CE2 | PHE | A | 169 | 40.786 | −4.521 | 0.141 | 1.00 | 30.08 | C |
| ATOM | 1299 | CD2 | PHE | A | 169 | 41.915 | −4.537 | −0.649 | 1.00 | 25.75 | C |
| ATOM | 1300 | C | PHE | A | 169 | 46.147 | −5.684 | −2.219 | 1.00 | 16.88 | C |
| ATOM | 1301 | O | PHE | A | 169 | 47.224 | −5.262 | −1.771 | 1.00 | 27.90 | O |
| ATOM | 1302 | N | SER | A | 170 | 45.995 | −5.971 | −3.497 | 1.00 | 15.05 | N |
| ATOM | 1303 | CA | SER | A | 170 | 47.153 | −5.864 | −4.375 | 1.00 | 19.37 | C |
| ATOM | 1304 | CB | SER | A | 170 | 47.693 | −7.264 | −4.707 | 1.00 | 17.07 | C |
| ATOM | 1305 | OG | SER | A | 170 | 47.485 | −8.185 | −3.621 | 1.00 | 31.38 | O |
| ATOM | 1306 | C | SER | A | 170 | 46.779 | −5.072 | −5.633 | 1.00 | 17.82 | C |
| ATOM | 1307 | O | SER | A | 170 | 45.632 | −5.084 | −6.049 | 1.00 | 25.53 | O |
| ATOM | 1308 | N | ALA | A | 171 | 47.722 | −4.319 | −6.179 | 1.00 | 19.21 | N |
| ATOM | 1309 | CA | ALA | A | 171 | 47.589 | −3.695 | −7.503 | 1.00 | 27.44 | C |
| ATOM | 1310 | CB | ALA | A | 171 | 46.968 | −2.320 | −7.406 | 1.00 | 16.04 | C |
| ATOM | 1311 | C | ALA | A | 171 | 48.940 | −3.587 | −8.231 | 1.00 | 38.10 | C |
| ATOM | 1312 | O | ALA | A | 171 | 50.019 | −3.560 | −7.598 | 1.00 | 33.38 | O |
| ATOM | 1313 | N | SER | A | 172 | 48.870 | −3.506 | −9.560 | 1.00 | 37.54 | N |
| ATOM | 1314 | CA | SER | A | 172 | 50.075 | −3.466 | −10.384 | 1.00 | 37.17 | C |
| ATOM | 1315 | CB | SER | A | 172 | 50.476 | −4.867 | −10.815 | 1.00 | 46.68 | C |
| ATOM | 1316 | OG | SER | A | 172 | 50.493 | −5.765 | −9.710 | 1.00 | 62.62 | O |
| ATOM | 1317 | C | SER | A | 172 | 49.821 | −2.661 | −11.632 | 1.00 | 42.13 | C |
| ATOM | 1318 | O | SER | A | 172 | 48.733 | −2.709 | −12.204 | 1.00 | 54.44 | O |
| ATOM | 1319 | N | GLY | A | 173 | 50.819 | −1.887 | −12.029 | 1.00 | 38.05 | N |
| ATOM | 1320 | CA | GLY | A | 173 | 50.568 | −0.740 | −12.890 | 1.00 | 37.53 | C |
| ATOM | 1321 | C | GLY | A | 173 | 51.846 | −0.298 | −13.565 | 1.00 | 40.98 | C |
| ATOM | 1322 | O | GLY | A | 173 | 52.672 | −1.128 | −13.907 | 1.00 | 52.75 | O |
| ATOM | 1323 | N | GLU | A | 174 | 51.984 | 0.996 | −13.824 | 1.00 | 47.54 | N |
| ATOM | 1324 | CA | GLU | A | 174 | 53.034 | 1.464 | −14.716 | 1.00 | 50.19 | C |
| ATOM | 1325 | CB | GLU | A | 174 | 52.610 | 2.759 | −15.416 | 1.00 | 59.72 | C |
| ATOM | 1326 | CG | GLU | A | 174 | 53.605 | 3.257 | −16.458 | 1.00 | 65.72 | C |
| ATOM | 1327 | CD | GLU | A | 174 | 53.674 | 2.343 | −17.664 | 1.00 | 91.89 | C |
| ATOM | 1328 | OE1 | GLU | A | 174 | 54.562 | 1.462 | −17.692 | 1.00 | 103.65 | O |
| ATOM | 1329 | OE2 | GLU | A | 174 | 52.807 | 2.472 | −18.557 | 1.00 | 105.23 | O |
| ATOM | 1330 | C | GLU | A | 174 | 54.357 | 1.653 | −13.968 | 1.00 | 44.74 | C |
| ATOM | 1331 | O | GLU | A | 174 | 55.397 | 1.125 | −14.382 | 1.00 | 26.27 | O |
| ATOM | 1332 | N | LEU | A | 175 | 54.282 | 2.334 | −12.824 | 1.00 | 45.82 | N |
| ATOM | 1333 | CA | LEU | A | 175 | 55.462 | 2.595 | −11.997 | 1.00 | 47.99 | C |
| ATOM | 1334 | CB | LEU | A | 175 | 55.266 | 3.844 | −11.138 | 1.00 | 45.05 | C |
| ATOM | 1335 | CG | LEU | A | 175 | 53.895 | 4.107 | −10.514 | 1.00 | 49.18 | C |
| ATOM | 1336 | CD1 | LEU | A | 175 | 53.959 | 3.919 | −8.984 | 1.00 | 42.92 | C |
| ATOM | 1337 | CD2 | LEU | A | 175 | 53.415 | 5.511 | −10.858 | 1.00 | 35.81 | C |
| ATOM | 1338 | C | LEU | A | 175 | 55.955 | 1.424 | −11.128 | 1.00 | 49.31 | C |
| ATOM | 1339 | O | LEU | A | 175 | 57.099 | 1.435 | −10.654 | 1.00 | 39.91 | O |
| ATOM | 1340 | N | GLY | A | 176 | 55.128 | 0.387 | −10.976 | 1.00 | 49.12 | N |
| ATOM | 1341 | CA | GLY | A | 176 | 55.561 | −0.831 | −10.303 | 1.00 | 46.21 | C |
| ATOM | 1342 | C | GLY | A | 176 | 54.380 | −1.649 | −9.826 | 1.00 | 45.99 | C |
| ATOM | 1343 | O | GLY | A | 176 | 53.365 | −1.745 | −10.530 | 1.00 | 55.11 | O |
| ATOM | 1344 | N | ASN | A | 177 | 54.473 | −2.164 | −8.602 | 1.00 | 36.00 | N |
| ATOM | 1345 | CA | ASN | A | 177 | 53.289 | −2.694 | −7.914 | 1.00 | 40.71 | C |
| ATOM | 1346 | CB | ASN | A | 177 | 53.062 | −4.160 | −8.281 | 1.00 | 40.34 | C |
| ATOM | 1347 | CG | ASN | A | 177 | 54.222 | −5.002 | −7.916 | 1.00 | 39.93 | C |
| ATOM | 1348 | OD1 | ASN | A | 177 | 54.369 | −5.394 | −6.745 | 1.00 | 50.29 | O |
| ATOM | 1349 | ND2 | ASN | A | 177 | 55.166 | −5.100 | −8.845 | 1.00 | 36.30 | N |
| ATOM | 1350 | C | ASN | A | 177 | 53.231 | −2.505 | −6.385 | 1.00 | 43.88 | C |
| ATOM | 1351 | O | ASN | A | 177 | 54.185 | −2.015 | −5.760 | 1.00 | 46.32 | O |
| ATOM | 1352 | N | GLY | A | 178 | 52.076 | −2.795 | −5.792 | 1.00 | 34.74 | N |
| ATOM | 1353 | CA | GLY | A | 178 | 51.909 | −2.474 | −4.393 | 1.00 | 25.44 | C |
| ATOM | 1354 | C | GLY | A | 178 | 51.111 | −3.575 | −3.733 | 1.00 | 30.16 | C |
| ATOM | 1355 | O | GLY | A | 178 | 50.214 | −4.139 | −4.356 | 1.00 | 25.55 | O |
| ATOM | 1356 | N | ASN | A | 179 | 51.389 | −3.797 | −2.450 | 1.00 | 34.85 | N |
| ATOM | 1357 | CA | ASN | A | 179 | 50.604 | −4.674 | −1.589 | 1.00 | 42.28 | C |
| ATOM | 1358 | CB | ASN | A | 179 | 51.325 | −6.006 | −1.411 | 1.00 | 39.10 | C |
| ATOM | 1359 | CG | ASN | A | 179 | 50.996 | −6.962 | −2.499 | 1.00 | 51.66 | C |
| ATOM | 1360 | OD1 | ASN | A | 179 | 50.186 | −7.868 | −2.300 | 1.00 | 70.96 | O |
| ATOM | 1361 | ND2 | ASN | A | 179 | 51.543 | −6.717 | −3.697 | 1.00 | 67.18 | N |
| ATOM | 1362 | C | ASN | A | 179 | 50.289 | −4.068 | −0.215 | 1.00 | 42.98 | C |
| ATOM | 1363 | O | ASN | A | 179 | 51.189 | −3.725 | 0.559 | 1.00 | 46.27 | O |
| ATOM | 1364 | N | ILE | A | 180 | 49.008 | −4.110 | 0.144 | 1.00 | 36.77 | N |
| ATOM | 1365 | CA | ILE | A | 180 | 48.561 | −3.753 | 1.487 | 1.00 | 30.59 | C |
| ATOM | 1366 | CB | ILE | A | 180 | 47.567 | −2.625 | 1.349 | 1.00 | 24.55 | C |
| ATOM | 1367 | CG1 | ILE | A | 180 | 48.228 | −1.507 | 0.541 | 1.00 | 40.53 | C |
| ATOM | 1368 | CD1 | ILE | A | 180 | 47.259 | −0.648 | −0.252 | 1.00 | 46.96 | C |
| ATOM | 1369 | CG2 | ILE | A | 180 | 47.150 | −2.122 | 2.699 | 1.00 | 32.24 | C |
| ATOM | 1370 | C | ILE | A | 180 | 47.944 | −4.889 | 2.322 | 1.00 | 35.63 | C |
| ATOM | 1371 | O | ILE | A | 180 | 46.767 | −5.223 | 2.159 | 1.00 | 40.70 | O |
| ATOM | 1372 | N | LYS | A | 181 | 48.735 | −5.455 | 3.234 | 1.00 | 39.04 | N |
| ATOM | 1373 | CA | LYS | A | 181 | 48.326 | −6.617 | 4.015 | 1.00 | 37.60 | C |
| ATOM | 1374 | CB | LYS | A | 181 | 49.545 | −7.505 | 4.342 | 1.00 | 33.55 | C |
| ATOM | 1375 | CG | LYS | A | 181 | 49.512 | −8.911 | 3.757 | 1.00 | 53.57 | C |
| ATOM | 1376 | CD | LYS | A | 181 | 48.759 | −9.918 | 4.637 | 1.00 | 79.19 | C |
| ATOM | 1377 | CE | LYS | A | 181 | 48.403 | −11.221 | 3.885 | 1.00 | 78.00 | C |
| ATOM | 1378 | NZ | LYS | A | 181 | 47.231 | −11.920 | 4.514 | 1.00 | 73.68 | N |
| ATOM | 1379 | C | LYS | A | 181 | 47.715 | −6.070 | 5.300 | 1.00 | 43.11 | C |
| ATOM | 1380 | O | LYS | A | 181 | 48.372 | −5.366 | 6.072 | 1.00 | 46.81 | O |
| ATOM | 1381 | N | LEU | A | 182 | 46.444 | −6.364 | 5.527 | 1.00 | 43.28 | N |
| ATOM | 1382 | CA | LEU | A | 182 | 45.805 | −5.912 | 6.749 | 1.00 | 38.01 | C |
| ATOM | 1383 | CB | LEU | A | 182 | 44.379 | −5.425 | 6.463 | 1.00 | 40.02 | C |
| ATOM | 1384 | CG | LEU | A | 182 | 44.193 | −4.113 | 5.685 | 1.00 | 25.23 | C |
| ATOM | 1385 | CD1 | LEU | A | 182 | 43.025 | −3.304 | 6.225 | 1.00 | 23.88 | C |
| ATOM | 1386 | CD2 | LEU | A | 182 | 45.455 | −3.317 | 5.802 | 1.00 | 36.42 | C |
| ATOM | 1387 | C | LEU | A | 182 | 45.761 | −7.092 | 7.683 | 1.00 | 43.49 | C |
| ATOM | 1388 | O | LEU | A | 182 | 45.042 | −8.038 | 7.430 | 1.00 | 54.18 | O |
| ATOM | 1389 | N | SER | A | 183 | 46.519 | −7.023 | 8.768 | 1.00 | 51.84 | N |
| ATOM | 1390 | CA | SER | A | 183 | 46.535 | −8.070 | 9.794 | 1.00 | 57.74 | C |
| ATOM | 1391 | CB | SER | A | 183 | 47.987 | −8.452 | 10.169 | 1.00 | 58.20 | C |
| ATOM | 1392 | OG | SER | A | 183 | 48.318 | −9.808 | 9.844 | 1.00 | 62.48 | O |
| ATOM | 1393 | C | SER | A | 183 | 45.708 | −7.693 | 11.045 | 1.00 | 59.03 | C |
| ATOM | 1394 | O | SER | A | 183 | 45.187 | −6.586 | 11.150 | 1.00 | 50.87 | O |
| ATOM | 1395 | N | GLN | A | 184 | 45.506 | −8.645 | 11.955 | 1.00 | 69.08 | N |
| ATOM | 1396 | CA | GLN | A | 184 | 44.941 | −8.332 | 13.255 | 1.00 | 67.21 | C |
| ATOM | 1397 | CB | GLN | A | 184 | 43.764 | −9.240 | 13.558 | 1.00 | 70.36 | C |
| ATOM | 1398 | CG | GLN | A | 184 | 43.012 | −8.806 | 14.803 | 1.00 | 84.87 | C |
| ATOM | 1399 | CD | GLN | A | 184 | 41.540 | −9.173 | 14.734 | 1.00 | 94.99 | C |
| ATOM | 1400 | OE1 | GLN | A | 184 | 40.711 | −8.619 | 15.461 | 1.00 | 87.73 | O |
| ATOM | 1401 | NE2 | GLN | A | 184 | 41.208 | −10.092 | 13.831 | 1.00 | 88.49 | N |
| ATOM | 1402 | C | GLN | A | 184 | 46.007 | −8.546 | 14.297 | 1.00 | 67.34 | C |
| ATOM | 1403 | O | GLN | A | 184 | 46.318 | −9.693 | 14.651 | 1.00 | 64.35 | O |
| ATOM | 1404 | N | THR | A | 185 | 48.604 | −7.439 | 14.745 | 1.00 | 80.27 | N |
| ATOM | 1405 | CA | THR | A | 185 | 47.799 | −7.523 | 15.568 | 1.00 | 87.82 | C |
| ATOM | 1406 | CB | THR | A | 185 | 48.763 | −6.408 | 15.174 | 1.00 | 86.27 | C |
| ATOM | 1407 | OG1 | THR | A | 185 | 50.034 | −6.617 | 15.812 | 1.00 | 73.75 | O |
| ATOM | 1408 | CG2 | THR | A | 185 | 48.279 | −5.103 | 15.766 | 1.00 | 79.74 | C |
| ATOM | 1409 | C | THR | A | 185 | 47.377 | −7.337 | 17.012 | 1.00 | 96.55 | C |
| ATOM | 1410 | O | THR | A | 185 | 48.266 | −6.895 | 17.284 | 1.00 | 100.14 | O |
| ATOM | 1411 | N | SER | A | 186 | 48.274 | −7.688 | 17.926 | 1.00 | 103.34 | N |
| ATOM | 1412 | CA | SER | A | 186 | 47.955 | −7.746 | 19.340 | 1.00 | 108.49 | C |
| ATOM | 1413 | CB | SER | A | 186 | 48.379 | −9.096 | 19.915 | 1.00 | 111.19 | C |
| ATOM | 1414 | OG | SER | A | 186 | 48.038 | −9.179 | 21.309 | 1.00 | 113.32 | O |
| ATOM | 1415 | C | SER | A | 186 | 48.684 | −6.639 | 20.071 | 1.00 | 106.98 | C |
| ATOM | 1416 | O | SER | A | 186 | 49.851 | −6.773 | 20.416 | 1.00 | 107.57 | O |
| ATOM | 1417 | N | ASN | A | 187 | 47.970 | −5.552 | 20.334 | 1.00 | 101.73 | N |
| ATOM | 1418 | CA | ASN | A | 187 | 48.579 | −4.368 | 20.919 | 1.00 | 96.89 | C |
| ATOM | 1419 | CB | ASN | A | 187 | 48.683 | −3.270 | 19.860 | 1.00 | 86.21 | C |
| ATOM | 1420 | CG | ASN | A | 187 | 47.327 | −2.881 | 19.292 | 1.00 | 77.77 | C |
| ATOM | 1421 | OD1 | ASN | A | 187 | 48.476 | −3.737 | 19.032 | 1.00 | 79.96 | O |
| ATOM | 1422 | ND2 | ASN | A | 187 | 47.112 | −1.574 | 19.118 | 1.00 | 75.65 | N |
| ATOM | 1423 | C | ASN | A | 187 | 47.790 | −3.846 | 22.118 | 1.00 | 102.15 | C |
| ATOM | 1424 | O | ASN | A | 187 | 46.859 | −4.500 | 22.606 | 1.00 | 95.79 | O |
| ATOM | 1425 | N | VAL | A | 188 | 48.157 | −2.648 | 22.567 | 1.00 | 113.25 | N |
| ATOM | 1426 | CA | VAL | A | 188 | 47.446 | −1.989 | 23.650 | 1.00 | 119.26 | C |
| ATOM | 1427 | CB | VAL | A | 188 | 48.291 | −1.971 | 24.924 | 1.00 | 115.05 | C |
| ATOM | 1428 | CG1 | VAL | A | 188 | 49.069 | −3.260 | 25.063 | 1.00 | 100.77 | C |
| ATOM | 1429 | CG2 | VAL | A | 188 | 49.243 | −0.804 | 24.899 | 1.00 | 95.21 | C |
| ATOM | 1430 | C | VAL | A | 188 | 47.198 | −0.546 | 23.258 | 1.00 | 132.30 | C |
| ATOM | 1431 | O | VAL | A | 188 | 48.107 | 0.285 | 23.383 | 1.00 | 137.96 | O |
| ATOM | 1432 | N | ASP | A | 189 | 45.982 | −0.260 | 22.739 | 1.00 | 141.83 | N |
| ATOM | 1433 | CA | ASP | A | 189 | 45.491 | 1.134 | 22.819 | 1.00 | 145.64 | C |
| ATOM | 1434 | CB | ASP | A | 189 | 45.554 | 1.834 | 21.435 | 1.00 | 144.90 | C |
| ATOM | 1435 | CG | ASP | A | 189 | 45.417 | 0.844 | 20.286 | 1.00 | 142.19 | C |
| ATOM | 1436 | OD1 | ASP | A | 189 | 44.833 | −0.248 | 20.504 | 1.00 | 134.78 | O |
| ATOM | 1437 | OD2 | ASP | A | 189 | 45.876 | 1.059 | 19.136 | 1.00 | 134.94 | O |
| ATOM | 1438 | C | ASP | A | 189 | 44.061 | 1.132 | 23.347 | 1.00 | 148.16 | C |
| ATOM | 1439 | O | ASP | A | 189 | 43.576 | 0.061 | 23.816 | 1.00 | 147.82 | O |
| ATOM | 1440 | N | LYS | A | 190 | 43.384 | 2.339 | 23.307 | 1.00 | 149.63 | N |
| ATOM | 1441 | CA | LYS | A | 190 | 41.903 | 2.197 | 23.309 | 1.00 | 149.43 | C |
| ATOM | 1442 | CB | LYS | A | 190 | 41.226 | 3.402 | 24.120 | 1.00 | 148.44 | C |
| ATOM | 1443 | CG | LYS | A | 190 | 40.203 | 4.375 | 23.285 | 1.00 | 144.49 | C |
| ATOM | 1444 | CD | LYS | A | 190 | 41.146 | 5.359 | 22.478 | 1.00 | 140.50 | C |
| ATOM | 1445 | CE | LYS | A | 190 | 41.789 | 4.520 | 21.318 | 1.00 | 136.29 | C |
| ATOM | 1446 | NZ | LYS | A | 190 | 41.528 | 5.211 | 20.004 | 1.00 | 127.73 | N |
| ATOM | 1447 | C | LYS | A | 190 | 41.438 | 2.068 | 21.813 | 1.00 | 148.82 | C |
| ATOM | 1448 | O | LYS | A | 190 | 42.126 | 1.484 | 20.907 | 1.00 | 148.89 | O |
| ATOM | 1449 | N | GLU | A | 191 | 40.227 | 2.560 | 21.569 | 1.00 | 145.06 | N |
| ATOM | 1450 | CA | GLU | A | 191 | 39.440 | 1.942 | 20.516 | 1.00 | 140.15 | C |
| ATOM | 1451 | CB | GLU | A | 191 | 37.921 | 1.913 | 20.890 | 1.00 | 137.83 | C |
| ATOM | 1452 | CG | GLU | A | 191 | 37.180 | 0.780 | 20.152 | 1.00 | 129.72 | C |
| ATOM | 1453 | CD | GLU | A | 191 | 36.771 | 1.219 | 18.756 | 1.00 | 129.35 | C |
| ATOM | 1454 | OE1 | GLU | A | 191 | 37.008 | 0.478 | 17.765 | 1.00 | 122.41 | O |
| ATOM | 1455 | OE2 | GLU | A | 191 | 36.240 | 2.350 | 18.652 | 1.00 | 126.03 | O |
| ATOM | 1456 | C | GLU | A | 191 | 39.690 | 2.617 | 19.159 | 1.00 | 138.68 | C |
| ATOM | 1457 | O | GLU | A | 191 | 39.029 | 3.608 | 18.804 | 1.00 | 136.54 | O |
| ATOM | 1458 | N | GLU | A | 192 | 40.632 | 2.035 | 18.395 | 1.00 | 133.97 | N |
| ATOM | 1459 | CA | GLU | A | 192 | 48.891 | 2.436 | 17.003 | 1.00 | 121.85 | C |
| ATOM | 1460 | CB | GLU | A | 192 | 42.347 | 2.940 | 16.876 | 1.00 | 120.62 | C |
| ATOM | 1461 | CG | GLU | A | 192 | 42.403 | 4.483 | 17.258 | 1.00 | 120.16 | C |
| ATOM | 1462 | CD | GLU | A | 192 | 41.195 | 5.254 | 16.740 | 1.00 | 124.27 | C |
| ATOM | 1463 | OE1 | GLU | A | 192 | 40.123 | 5.304 | 17.468 | 1.00 | 122.63 | O |
| ATOM | 1464 | OE2 | GLU | A | 192 | 41.326 | 5.804 | 15.589 | 1.00 | 121.42 | O |
| ATOM | 1465 | C | GLU | A | 192 | 40.624 | 1.302 | 16.012 | 1.00 | 109.57 | C |
| ATOM | 1466 | O | GLU | A | 192 | 41.079 | 1.309 | 14.873 | 1.00 | 107.24 | O |
| ATOM | 1467 | N | GLU | A | 193 | 39.843 | 0.331 | 16.448 | 1.00 | 92.87 | N |
| ATOM | 1468 | CA | GLU | A | 193 | 39.328 | −0.648 | 15.521 | 1.00 | 86.92 | C |
| ATOM | 1469 | CB | GLU | A | 193 | 38.518 | 0.027 | 14.428 | 1.00 | 90.18 | C |
| ATOM | 1470 | CG | GLU | A | 193 | 37.708 | 1.215 | 14.912 | 1.00 | 101.18 | C |
| ATOM | 1471 | CD | GLU | A | 193 | 37.891 | 2.424 | 14.013 | 1.00 | 117.52 | C |
| ATOM | 1472 | OE1 | GLU | A | 193 | 36.945 | 3.227 | 13.899 | 1.00 | 118.52 | O |
| ATOM | 1473 | OE2 | GLU | A | 193 | 38.974 | 2.564 | 13.414 | 1.00 | 116.17 | O |
| ATOM | 1474 | C | GLU | A | 193 | 40.465 | −1.311 | 14.823 | 1.00 | 79.69 | C |
| ATOM | 1475 | O | GLU | A | 193 | 41.603 | −0.876 | 14.900 | 1.00 | 83.62 | O |
| ATOM | 1476 | N | ALA | A | 194 | 40.098 | −2.276 | 14.004 | 1.00 | 78.84 | N |
| ATOM | 1477 | CA | ALA | A | 194 | 41.059 | −3.279 | 13.540 | 1.00 | 86.04 | C |
| ATOM | 1478 | CB | ALA | A | 194 | 41.110 | −4.460 | 14.578 | 1.00 | 85.02 | C |
| ATOM | 1479 | C | ALA | A | 194 | 40.557 | −3.757 | 12.180 | 1.00 | 85.17 | C |
| ATOM | 1480 | O | ALA | A | 194 | 40.340 | −2.934 | 11.236 | 1.00 | 89.83 | O |
| ATOM | 1481 | N | VAL | A | 195 | 40.279 | −5.096 | 12.129 | 1.00 | 77.56 | N |
| ATOM | 1482 | CA | VAL | A | 195 | 39.678 | −5.651 | 10.933 | 1.00 | 65.00 | C |
| ATOM | 1483 | CB | VAL | A | 195 | 40.714 | −5.756 | 9.806 | 1.00 | 66.04 | C |
| ATOM | 1484 | CG1 | VAL | A | 195 | 40.318 | −6.827 | 8.797 | 1.00 | 60.00 | C |
| ATOM | 1485 | CG2 | VAL | A | 195 | 40.871 | −4.419 | 9.141 | 1.00 | 55.19 | C |
| ATOM | 1486 | C | VAL | A | 195 | 39.244 | −7.048 | 11.298 | 1.00 | 63.76 | C |
| ATOM | 1487 | O | VAL | A | 195 | 39.995 | −8.005 | 11.138 | 1.00 | 77.48 | O |
| ATOM | 1488 | N | THR | A | 196 | 38.026 | −7.175 | 11.788 | 1.00 | 59.85 | N |
| ATOM | 1489 | CA | THR | A | 196 | 37.499 | −8.490 | 12.071 | 1.00 | 62.50 | C |
| ATOM | 1490 | CB | THR | A | 196 | 36.519 | −8.350 | 13.248 | 1.00 | 59.92 | C |
| ATOM | 1491 | OG1 | THR | A | 196 | 35.728 | −9.534 | 13.398 | 1.00 | 79.54 | O |
| ATOM | 1492 | CG2 | THR | A | 196 | 35.520 | −7.266 | 12.980 | 1.00 | 51.29 | C |
| ATOM | 1493 | C | THR | A | 196 | 36.913 | −9.161 | 10.808 | 1.00 | 64.88 | C |
| ATOM | 1494 | O | THR | A | 196 | 36.419 | −8.466 | 9.913 | 1.00 | 59.27 | O |
| ATOM | 1495 | N | ILE | A | 197 | 37.106 | −10.481 | 10.670 | 1.00 | 69.94 | N |
| ATOM | 1496 | CA | ILE | A | 197 | 36.582 | −11.259 | 9.538 | 1.00 | 61.64 | C |
| ATOM | 1497 | CB | ILE | A | 197 | 37.725 | −11.650 | 8.577 | 1.00 | 58.35 | C |
| ATOM | 1498 | CG1 | ILE | A | 197 | 38.987 | −12.047 | 9.357 | 1.00 | 61.69 | C |
| ATOM | 1499 | CD1 | ILE | A | 197 | 40.241 | −11.257 | 8.988 | 1.00 | 63.17 | C |
| ATOM | 1500 | CG2 | ILE | A | 197 | 38.001 | −10.491 | 7.630 | 1.00 | 69.73 | C |
| ATOM | 1501 | C | ILE | A | 197 | 35.814 | −12.500 | 9.987 | 1.00 | 52.50 | C |
| ATOM | 1502 | O | ILE | A | 197 | 36.235 | −13.182 | 10.907 | 1.00 | 55.60 | O |
| ATOM | 1503 | N | GLU | A | 198 | 34.712 | −12.819 | 9.320 | 1.00 | 47.90 | N |
| ATOM | 1504 | CA | GLU | A | 198 | 34.099 | −14.133 | 9.484 | 1.00 | 51.27 | C |
| ATOM | 1505 | CB | GLU | A | 198 | 32.605 | −13.984 | 9.699 | 1.00 | 49.60 | C |
| ATOM | 1506 | CG | GLU | A | 198 | 32.220 | −12.612 | 10.207 | 1.00 | 72.16 | C |
| ATOM | 1507 | CD | GLU | A | 198 | 31.660 | −12.662 | 11.611 | 1.00 | 93.79 | C |
| ATOM | 1508 | OE1 | GLU | A | 198 | 32.379 | −12.239 | 12.541 | 1.00 | 92.64 | O |
| ATOM | 1509 | OE2 | GLU | A | 198 | 30.510 | −13.130 | 11.781 | 1.00 | 99.30 | O |
| ATOM | 1510 | C | GLU | A | 198 | 34.310 | −15.008 | 8.260 | 1.00 | 54.20 | C |
| ATOM | 1511 | O | GLU | A | 198 | 33.369 | −15.293 | 7.534 | 1.00 | 58.06 | O |
| ATOM | 1512 | N | MET | A | 199 | 35.539 | −15.452 | 8.043 | 1.00 | 54.44 | N |
| ATOM | 1513 | CA | MET | A | 199 | 35.864 | −16.251 | 6.874 | 1.00 | 51.52 | C |
| ATOM | 1514 | CB | MET | A | 199 | 37.385 | −16.266 | 6.686 | 1.00 | 43.29 | C |
| ATOM | 1515 | CG | MET | A | 199 | 37.891 | −17.222 | 5.621 | 1.00 | 48.58 | C |
| ATOM | 1516 | SD | MET | A | 199 | 39.095 | −18.365 | 6.274 | 1.00 | 94.74 | S |
| ATOM | 1517 | CE | MET | A | 199 | 38.356 | −19.963 | 5.826 | 1.00 | 82.69 | C |
| ATOM | 1518 | C | MET | A | 199 | 35.315 | −17.670 | 7.061 | 1.00 | 57.70 | C |
| ATOM | 1519 | O | MET | A | 199 | 35.706 | −18.381 | 7.984 | 1.00 | 61.08 | O |
| ATOM | 1520 | N | ASN | A | 200 | 34.426 | −18.094 | 6.168 | 1.00 | 64.29 | N |
| ATOM | 1521 | CA | ASN | A | 200 | 34.176 | −19.525 | 5.986 | 1.00 | 73.57 | C |
| ATOM | 1522 | CB | ASN | A | 200 | 32.704 | −19.800 | 5.644 | 1.00 | 76.92 | C |
| ATOM | 1523 | CG | ASN | A | 200 | 31.754 | −19.245 | 6.687 | 1.00 | 87.56 | C |
| ATOM | 1524 | OD1 | ASN | A | 200 | 32.191 | −18.710 | 7.712 | 1.00 | 85.90 | O |
| ATOM | 1525 | ND2 | ASN | A | 200 | 30.449 | −19.345 | 6.419 | 1.00 | 89.52 | N |
| ATOM | 1526 | C | ASN | A | 200 | 35.100 | −20.247 | 5.001 | 1.00 | 73.22 | C |
| ATOM | 1527 | O | ASN | A | 200 | 35.470 | −21.402 | 5.227 | 1.00 | 84.74 | O |
| ATOM | 1528 | N | GLU | A | 201 | 35.393 | −19.611 | 3.871 | 1.00 | 64.48 | N |
| ATOM | 1529 | CA | GLU | A | 201 | 36.357 | −20.151 | 2.928 | 1.00 | 53.40 | C |
| ATOM | 1530 | CB | GLU | A | 201 | 35.665 | −20.917 | 1.789 | 1.00 | 54.74 | C |
| ATOM | 1531 | CG | GLU | A | 201 | 34.182 | −20.600 | 1.640 | 1.00 | 70.40 | C |
| ATOM | 1532 | CD | GLU | A | 201 | 33.578 | −21.186 | 0.374 | 1.00 | 90.38 | C |
| ATOM | 1533 | OE1 | GLU | A | 201 | 32.335 | −21.223 | 0.276 | 1.00 | 89.70 | O |
| ATOM | 1534 | OE2 | GLU | A | 201 | 34.334 | −21.584 | −0.539 | 1.00 | 86.45 | O |
| ATOM | 1535 | C | GLU | A | 201 | 37.102 | −18.973 | 2.370 | 1.00 | 48.82 | C |
| ATOM | 1536 | O | GLU | A | 201 | 36.500 | −17.940 | 2.080 | 1.00 | 46.68 | O |
| ATOM | 1537 | N | PRO | A | 202 | 38.398 | −19.162 | 2.165 | 1.00 | 45.96 | N |
| ATOM | 1538 | CA | PRO | A | 202 | 39.259 | −18.166 | 1.510 | 1.00 | 51.93 | C |
| ATOM | 1539 | CB | PRO | A | 202 | 40.611 | −18.879 | 1.446 | 1.00 | 52.08 | C |
| ATOM | 1540 | CG | PRO | A | 202 | 40.532 | −19.912 | 2.526 | 1.00 | 50.71 | C |
| ATOM | 1541 | CD | PRO | A | 202 | 39.125 | −20.388 | 2.529 | 1.00 | 40.33 | C |
| ATOM | 1542 | C | PRO | A | 202 | 38.819 | −17.769 | 0.093 | 1.00 | 54.22 | C |
| ATOM | 1543 | O | PRO | A | 202 | 38.642 | −18.665 | −0.746 | 1.00 | 62.60 | O |
| ATOM | 1544 | N | VAL | A | 203 | 38.668 | −16.463 | −0.160 | 1.00 | 50.14 | N |
| ATOM | 1545 | CA | VAL | A | 203 | 38.314 | −15.937 | −1.481 | 1.00 | 45.87 | C |
| ATOM | 1546 | CB | VAL | A | 203 | 37.001 | −15.194 | −1.468 | 1.00 | 37.51 | C |
| ATOM | 1547 | CG1 | VAL | A | 203 | 36.243 | −15.524 | −2.738 | 1.00 | 58.48 | C |
| ATOM | 1548 | CG2 | VAL | A | 203 | 36.184 | −15.601 | −0.272 | 1.00 | 48.98 | C |
| ATOM | 1549 | C | VAL | A | 203 | 39.358 | −15.027 | −2.115 | 1.00 | 46.83 | C |
| ATOM | 1550 | O | VAL | A | 203 | 40.159 | −14.408 | −1.423 | 1.00 | 59.39 | O |
| ATOM | 1551 | N | GLN | A | 204 | 39.396 | −14.999 | −3.440 | 1.00 | 38.52 | N |
| ATOM | 1552 | CA | GLN | A | 204 | 40.335 | −14.144 | −4.131 | 1.00 | 34.54 | C |
| ATOM | 1553 | CB | GLN | A | 204 | 41.646 | −14.882 | −4.398 | 1.00 | 32.98 | C |
| ATOM | 1554 | CG | GLN | A | 204 | 42.838 | −13.986 | −4.748 | 1.00 | 39.47 | C |
| ATOM | 1555 | CD | GLN | A | 204 | 43.961 | −14.739 | −5.464 | 1.00 | 51.54 | C |
| ATOM | 1556 | OE1 | GLN | A | 204 | 44.616 | −15.603 | −4.879 | 1.00 | 60.05 | O |
| ATOM | 1557 | NE2 | GLN | A | 204 | 44.162 | −14.431 | −6.733 | 1.00 | 53.95 | N |
| ATOM | 1558 | C | GLN | A | 204 | 39.661 | −13.727 | −5.424 | 1.00 | 38.62 | C |
| ATOM | 1559 | O | GLN | A | 204 | 39.516 | −14.528 | −6.352 | 1.00 | 45.67 | O |
| ATOM | 1560 | N | LEU | A | 205 | 39.209 | −12.477 | −5.462 | 1.00 | 33.16 | N |
| ATOM | 1561 | CA | LEU | A | 205 | 38.680 | −11.900 | −6.691 | 1.00 | 39.66 | C |
| ATOM | 1562 | CB | LEU | A | 205 | 37.175 | −11.701 | −6.556 | 1.00 | 45.31 | C |
| ATOM | 1563 | CG | LEU | A | 205 | 36.500 | −12.636 | −5.552 | 1.00 | 49.57 | C |
| ATOM | 1564 | CD1 | LEU | A | 205 | 36.271 | −11.880 | −4.254 | 1.00 | 69.96 | C |
| ATOM | 1565 | CD2 | LEU | A | 205 | 35.182 | −13.179 | −6.096 | 1.00 | 31.50 | C |
| ATOM | 1566 | C | LEU | A | 205 | 39.380 | −10.608 | −7.145 | 1.00 | 41.95 | C |
| ATOM | 1567 | O | LEU | A | 205 | 40.263 | −10.087 | −6.449 | 1.00 | 45.82 | O |
| ATOM | 1568 | N | THR | A | 206 | 39.027 | −10.113 | −8.331 | 1.00 | 37.34 | N |
| ATOM | 1569 | CA | THR | A | 206 | 39.555 | −8.832 | −8.803 | 1.00 | 34.77 | C |
| ATOM | 1570 | CB | THR | A | 206 | 40.412 | −9.037 | −10.079 | 1.00 | 35.01 | C |
| ATOM | 1571 | OG1 | THR | A | 206 | 41.358 | −10.088 | −9.838 | 1.00 | 38.71 | O |
| ATOM | 1572 | CG2 | THR | A | 206 | 41.306 | −7.816 | −10.370 | 1.00 | 26.41 | C |
| ATOM | 1573 | C | THR | A | 206 | 38.421 | −7.842 | −9.042 | 1.00 | 27.63 | C |
| ATOM | 1574 | O | THR | A | 206 | 37.363 | −8.224 | −9.527 | 1.00 | 35.00 | O |
| ATOM | 1575 | N | PHE | A | 207 | 38.606 | −6.590 | −8.633 | 1.00 | 26.46 | N |
| ATOM | 1576 | CA | PHE | A | 207 | 37.586 | −5.564 | −8.850 | 1.00 | 26.09 | C |
| ATOM | 1577 | CB | PHE | A | 207 | 36.869 | −5.224 | −7.542 | 1.00 | 24.72 | C |
| ATOM | 1578 | CG | PHE | A | 207 | 36.207 | −6.393 | −6.897 | 1.00 | 29.27 | C |
| ATOM | 1579 | CD1 | PHE | A | 207 | 34.956 | −6.821 | −7.317 | 1.00 | 27.88 | C |
| ATOM | 1580 | CE1 | PHE | A | 207 | 34.329 | −7.902 | −6.719 | 1.00 | 37.65 | C |
| ATOM | 1581 | CZ | PHE | A | 207 | 34.979 | −8.594 | −5.727 | 1.00 | 33.67 | C |
| ATOM | 1582 | CE2 | PHE | A | 207 | 36.214 | −8.140 | −5.265 | 1.00 | 54.48 | C |
| ATOM | 1583 | CD2 | PHE | A | 207 | 36.818 | −7.041 | −5.847 | 1.00 | 32.96 | C |
| ATOM | 1584 | C | PHE | A | 207 | 38.185 | −4.295 | −9.422 | 1.00 | 22.72 | C |
| ATOM | 1585 | O | PHE | A | 207 | 39.380 | −4.043 | −9.247 | 1.00 | 19.42 | O |
| ATOM | 1586 | N | ALA | A | 208 | 37.342 | −3.468 | −10.036 | 1.00 | 18.43 | N |
| ATOM | 1587 | CA | ALA | A | 208 | 37.759 | −2.135 | −10.493 | 1.00 | 21.20 | C |
| ATOM | 1588 | CB | ALA | A | 208 | 36.937 | −1.729 | −11.643 | 1.00 | 19.80 | C |
| ATOM | 1589 | C | ALA | A | 208 | 37.803 | −0.989 | −9.461 | 1.00 | 26.70 | C |
| ATOM | 1590 | O | ALA | A | 208 | 36.848 | −0.739 | −8.714 | 1.00 | 33.78 | O |
| ATOM | 1591 | N | LEU | A | 209 | 38.902 | −0.246 | −9.464 | 1.00 | 27.94 | N |
| ATOM | 1592 | CA | LEU | A | 209 | 39.156 | 0.720 | −8.407 | 1.00 | 30.72 | C |
| ATOM | 1593 | CB | LEU | A | 209 | 40.667 | 0.975 | −8.253 | 1.00 | 31.73 | C |
| ATOM | 1594 | CG | LEU | A | 209 | 41.532 | −0.060 | −7.515 | 1.00 | 33.26 | C |
| ATOM | 1595 | CD1 | LEU | A | 209 | 43.004 | 0.171 | −7.866 | 1.00 | 27.55 | C |
| ATOM | 1596 | CD2 | LEU | A | 209 | 41.315 | 0.008 | −6.015 | 1.00 | 42.30 | C |
| ATOM | 1597 | C | LEU | A | 209 | 38.392 | 2.008 | −8.729 | 1.00 | 32.25 | C |
| ATOM | 1598 | O | LEU | A | 209 | 38.081 | 2.819 | −7.842 | 1.00 | 32.00 | O |
| ATOM | 1599 | N | ARG | A | 210 | 38.092 | 2.190 | −10.011 | 1.00 | 31.77 | N |
| ATOM | 1600 | CA | ARG | A | 210 | 37.259 | 3.312 | −10.423 | 1.00 | 27.69 | C |
| ATOM | 1601 | CB | ARG | A | 210 | 36.967 | 3.254 | −11.932 | 1.00 | 21.75 | C |
| ATOM | 1602 | CG | ARG | A | 210 | 36.460 | 4.556 | −12.534 | 1.00 | 33.92 | C |
| ATOM | 1603 | CD | ARG | A | 210 | 36.487 | 4.604 | −14.057 | 1.00 | 65.99 | C |
| ATOM | 1604 | NE | ARG | A | 210 | 35.191 | 4.974 | −14.629 | 1.00 | 64.92 | N |
| ATOM | 1605 | CZ | ARG | A | 210 | 34.891 | 6.184 | −15.093 | 1.00 | 81.35 | C |
| ATOM | 1606 | NH1 | ARG | A | 210 | 35.777 | 7.180 | −15.018 | 1.00 | 91.00 | N |
| ATOM | 1607 | NH2 | ARG | A | 210 | 33.693 | 6.407 | −15.624 | 1.00 | 85.91 | N |
| ATOM | 1608 | C | ARG | A | 210 | 35.957 | 3.296 | −9.616 | 1.00 | 30.03 | C |
| ATOM | 1609 | O | ARG | A | 210 | 35.528 | 4.321 | −9.091 | 1.00 | 28.34 | O |
| ATOM | 1610 | N | TYR | A | 211 | 35.302 | 2.143 | −9.560 | 1.00 | 31.27 | N |
| ATOM | 1611 | CA | TYR | A | 211 | 33.963 | 2.091 | −9.016 | 1.00 | 26.71 | C |
| ATOM | 1612 | CB | TYR | A | 211 | 33.214 | 0.886 | −9.557 | 1.00 | 25.90 | C |
| ATOM | 1613 | CG | TYR | A | 211 | 32.806 | 1.087 | −10.996 | 1.00 | 33.09 | C |
| ATOM | 1614 | CD1 | TYR | A | 211 | 31.693 | 1.858 | −11.329 | 1.00 | 42.58 | C |
| ATOM | 1615 | CE1 | TYR | A | 211 | 31.292 | 2.014 | −12.650 | 1.00 | 32.82 | C |
| ATOM | 1616 | CZ | TYR | A | 211 | 32.026 | 1.430 | −13.643 | 1.00 | 27.85 | C |
| ATOM | 1617 | OH | TYR | A | 211 | 31.635 | 1.593 | −14.943 | 1.00 | 73.33 | O |
| ATOM | 1618 | CE2 | TYR | A | 211 | 33.151 | 0.679 | −13.345 | 1.00 | 38.84 | C |
| ATOM | 1619 | CD2 | TYR | A | 211 | 33.549 | 0.534 | −12.031 | 1.00 | 16.81 | C |
| ATOM | 1620 | C | TYR | A | 211 | 34.091 | 2.039 | −7.512 | 1.00 | 29.26 | C |
| ATOM | 1621 | O | TYR | A | 211 | 33.249 | 2.582 | −6.779 | 1.00 | 29.55 | O |
| ATOM | 1622 | N | LEU | A | 212 | 35.176 | 1.445 | −7.033 | 1.00 | 29.77 | N |
| ATOM | 1623 | CA | LEU | A | 212 | 35.393 | 1.431 | −5.586 | 1.00 | 21.99 | C |
| ATOM | 1624 | CB | LEU | A | 212 | 36.600 | 0.577 | −5.206 | 1.00 | 12.05 | C |
| ATOM | 1625 | CG | LEU | A | 212 | 36.281 | −0.903 | −4.978 | 1.00 | 23.19 | C |
| ATOM | 1626 | CD1 | LEU | A | 212 | 37.534 | −1.732 | −4.750 | 1.00 | 20.22 | C |
| ATOM | 1627 | CD2 | LEU | A | 212 | 35.293 | −1.071 | −3.844 | 1.00 | 22.05 | C |
| ATOM | 1628 | C | LEU | A | 212 | 35.583 | 2.850 | −5.077 | 1.00 | 22.37 | C |
| ATOM | 1629 | O | LEU | A | 212 | 35.245 | 3.165 | −3.939 | 1.00 | 24.61 | O |
| ATOM | 1630 | N | ASN | A | 213 | 36.134 | 3.710 | −5.925 | 1.00 | 18.90 | N |
| ATOM | 1631 | CA | ASN | A | 213 | 36.346 | 5.082 | −5.512 | 1.00 | 23.36 | C |
| ATOM | 1632 | CB | ASN | A | 213 | 37.319 | 5.780 | −6.457 | 1.00 | 29.22 | C |
| ATOM | 1633 | CG | ASN | A | 213 | 38.765 | 5.520 | −6.084 | 1.00 | 31.15 | C |
| ATOM | 1634 | OD1 | ASN | A | 213 | 39.035 | 4.916 | −5.053 | 1.00 | 33.45 | O |
| ATOM | 1635 | ND2 | ASN | A | 213 | 39.698 | 6.018 | −6.894 | 1.00 | 51.93 | N |
| ATOM | 1636 | C | ASN | A | 213 | 35.032 | 5.825 | −5.419 | 1.00 | 26.86 | C |
| ATOM | 1637 | O | ASN | A | 213 | 34.925 | 6.847 | −4.758 | 1.00 | 29.99 | O |
| ATOM | 1638 | N | PHE | A | 214 | 34.032 | 5.301 | −6.111 | 1.00 | 34.72 | N |
| ATOM | 1639 | CA | PHE | A | 214 | 32.723 | 5.938 | −6.146 | 1.00 | 35.28 | C |
| ATOM | 1640 | CB | PHE | A | 214 | 31.928 | 5.428 | −7.362 | 1.00 | 37.96 | C |
| ATOM | 1641 | CG | PHE | A | 214 | 32.344 | 6.056 | −8.660 | 1.00 | 38.12 | C |
| ATOM | 1642 | CD1 | PHE | A | 214 | 32.842 | 7.348 | −8.681 | 1.00 | 39.77 | C |
| ATOM | 1643 | CE1 | PHE | A | 214 | 33.228 | 7.936 | −9.867 | 1.00 | 56.61 | C |
| ATOM | 1644 | CZ | PHE | A | 214 | 33.149 | 7.218 | −11.043 | 1.00 | 50.73 | C |
| ATOM | 1645 | CE2 | PHE | A | 214 | 32.683 | 5.912 | −11.025 | 1.00 | 53.59 | C |
| ATOM | 1646 | CD2 | PHE | A | 214 | 32.286 | 5.340 | −9.842 | 1.00 | 55.74 | C |
| ATOM | 1647 | C | PHE | A | 214 | 32.034 | 5.544 | −4.858 | 1.00 | 28.91 | C |
| ATOM | 1648 | O | PHE | A | 214 | 31.156 | 6.227 | −4.345 | 1.00 | 22.45 | O |
| ATOM | 1649 | N | PHE | A | 215 | 32.437 | 4.390 | −4.360 | 1.00 | 36.15 | N |
| ATOM | 1650 | CA | PHE | A | 215 | 31.769 | 3.806 | −3.214 | 1.00 | 38.06 | C |
| ATOM | 1651 | CB | PHE | A | 215 | 32.121 | 2.322 | −3.159 | 1.00 | 39.04 | C |
| ATOM | 1652 | CG | PHE | A | 215 | 31.553 | 1.523 | −4.305 | 1.00 | 32.89 | C |
| ATOM | 1653 | CD1 | PHE | A | 215 | 30.567 | 2.074 | −5.119 | 1.00 | 22.55 | C |
| ATOM | 1654 | CE1 | PHE | A | 215 | 29.991 | 1.326 | −6.134 | 1.00 | 40.59 | C |
| ATOM | 1655 | CZ | PHE | A | 215 | 30.389 | −0.009 | −6.324 | 1.00 | 42.57 | C |
| ATOM | 1656 | CE2 | PHE | A | 215 | 31.342 | −0.582 | −5.476 | 1.00 | 12.96 | C |
| ATOM | 1657 | CD2 | PHE | A | 215 | 31.929 | 0.190 | −4.493 | 1.00 | 9.88 | C |
| ATOM | 1658 | C | PHE | A | 215 | 32.206 | 4.514 | −1.936 | 1.00 | 35.13 | C |
| ATOM | 1659 | O | PHE | A | 215 | 31.404 | 4.717 | −1.025 | 1.00 | 32.59 | O |
| ATOM | 1660 | N | THR | A | 216 | 33.492 | 4.860 | −1.876 | 1.00 | 29.11 | N |
| ATOM | 1661 | CA | THR | A | 216 | 34.053 | 5.528 | −0.713 | 1.00 | 25.79 | C |
| ATOM | 1662 | CB | THR | A | 216 | 35.589 | 5.535 | −0.794 | 1.00 | 12.35 | C |
| ATOM | 1663 | OG1 | THR | A | 216 | 35.996 | 6.002 | −2.079 | 1.00 | 27.11 | O |
| ATOM | 1664 | CG2 | THR | A | 216 | 36.099 | 4.140 | −0.805 | 1.00 | 18.21 | C |
| ATOM | 1665 | C | THR | A | 216 | 33.537 | 6.957 | −0.569 | 1.00 | 28.89 | C |
| ATOM | 1666 | O | THR | A | 216 | 34.052 | 7.734 | 0.237 | 1.00 | 34.83 | O |
| ATOM | 1667 | N | LYS | A | 217 | 32.576 | 7.335 | −1.399 | 1.00 | 20.01 | N |
| ATOM | 1668 | CA | LYS | A | 217 | 31.969 | 8.651 | −1.243 | 1.00 | 27.51 | C |
| ATOM | 1669 | CB | LYS | A | 217 | 31.388 | 9.140 | −2.564 | 1.00 | 24.05 | C |
| ATOM | 1670 | CG | LYS | A | 217 | 32.440 | 9.517 | −3.590 | 1.00 | 21.99 | C |
| ATOM | 1671 | CD | LYS | A | 217 | 32.855 | 10.970 | −3.407 | 1.00 | 48.88 | C |
| ATOM | 1672 | CE | LYS | A | 217 | 34.207 | 11.256 | −4.017 | 1.00 | 49.76 | C |
| ATOM | 1673 | NZ | LYS | A | 217 | 34.008 | 11.670 | −5.427 | 1.00 | 77.20 | N |
| ATOM | 1674 | C | LYS | A | 217 | 30.881 | 8.599 | −0.172 | 1.00 | 33.74 | C |
| ATOM | 1675 | O | LYS | A | 217 | 30.237 | 9.603 | 0.140 | 1.00 | 33.54 | O |
| ATOM | 1676 | N | ALA | A | 218 | 30.743 | 7.426 | 0.437 | 1.00 | 29.64 | N |
| ATOM | 1677 | CA | ALA | A | 218 | 29.765 | 7.241 | 1.477 | 1.00 | 22.90 | C |
| ATOM | 1678 | CB | ALA | A | 218 | 29.023 | 5.936 | 1.225 | 1.00 | 25.53 | C |
| ATOM | 1679 | C | ALA | A | 218 | 30.476 | 7.209 | 2.816 | 1.00 | 22.08 | C |
| ATOM | 1680 | O | ALA | A | 218 | 29.983 | 6.644 | 3.781 | 1.00 | 31.09 | O |
| ATOM | 1681 | N | THR | A | 219 | 31.657 | 7.792 | 2.883 | 1.00 | 17.94 | N |
| ATOM | 1682 | CA | THR | A | 219 | 32.475 | 7.617 | 4.080 | 1.00 | 26.66 | C |
| ATOM | 1683 | CB | THR | A | 219 | 33.917 | 8.039 | 3.838 | 1.00 | 23.52 | C |
| ATOM | 1684 | OG1 | THR | A | 219 | 34.562 | 7.063 | 3.007 | 1.00 | 35.90 | O |
| ATOM | 1685 | CG2 | THR | A | 219 | 34.694 | 7.966 | 5.115 | 1.00 | 24.20 | C |
| ATOM | 1686 | C | THR | A | 219 | 31.911 | 8.382 | 5.251 | 1.00 | 22.86 | C |
| ATOM | 1687 | O | THR | A | 219 | 31.850 | 7.884 | 6.355 | 1.00 | 30.46 | O |
| ATOM | 1688 | N | PRO | A | 220 | 31.509 | 9.611 | 5.021 | 1.00 | 26.82 | N |
| ATOM | 1689 | CA | PRO | A | 220 | 31.006 | 10.439 | 6.108 | 1.00 | 34.68 | C |
| ATOM | 1690 | CB | PRO | A | 220 | 30.675 | 11.756 | 5.392 | 1.00 | 36.65 | C |
| ATOM | 1691 | CG | PRO | A | 220 | 31.551 | 11.743 | 4.202 | 1.00 | 15.32 | C |
| ATOM | 1692 | CD | PRO | A | 220 | 31.517 | 10.331 | 3.744 | 1.00 | 31.84 | C |
| ATOM | 1693 | C | PRO | A | 220 | 29.766 | 9.809 | 6.747 | 1.00 | 34.39 | C |
| ATOM | 1694 | O | PRO | A | 220 | 29.354 | 10.269 | 7.820 | 1.00 | 39.27 | O |
| ATOM | 1695 | N | LEU | A | 221 | 29.176 | 8.807 | 6.089 | 1.00 | 24.83 | N |
| ATOM | 1696 | CA | LEU | A | 221 | 27.878 | 8.274 | 6.515 | 1.00 | 24.36 | C |
| ATOM | 1697 | CB | LEU | A | 221 | 27.158 | 7.576 | 5.365 | 1.00 | 11.56 | C |
| ATOM | 1698 | CG | LEU | A | 221 | 26.195 | 8.473 | 4.572 | 1.00 | 32.42 | C |
| ATOM | 1699 | CD1 | LEU | A | 221 | 25.709 | 7.828 | 3.294 | 1.00 | 26.41 | C |
| ATOM | 1700 | CD2 | LEU | A | 221 | 25.001 | 8.892 | 5.414 | 1.00 | 62.13 | C |
| ATOM | 1701 | C | LEU | A | 221 | 28.002 | 7.330 | 7.700 | 1.00 | 29.04 | C |
| ATOM | 1702 | O | LEU | A | 221 | 27.134 | 7.292 | 8.556 | 1.00 | 34.45 | O |
| ATOM | 1703 | N | SER | A | 222 | 29.131 | 6.621 | 7.770 | 1.00 | 36.02 | N |
| ATOM | 1704 | CA | SER | A | 222 | 29.323 | 5.543 | 8.730 | 1.00 | 38.24 | C |
| ATOM | 1705 | CB | SER | A | 222 | 28.997 | 4.213 | 8.041 | 1.00 | 44.85 | C |
| ATOM | 1706 | OG | SER | A | 222 | 28.294 | 3.327 | 8.901 | 1.00 | 51.00 | O |
| ATOM | 1707 | C | SER | A | 222 | 30.781 | 5.557 | 9.219 | 1.00 | 42.79 | C |
| ATOM | 1708 | O | SER | A | 222 | 31.675 | 6.064 | 8.545 | 1.00 | 45.84 | O |
| ATOM | 1709 | N | SER | A | 223 | 31.045 | 5.005 | 10.396 | 1.00 | 44.87 | N |
| ATOM | 1710 | CA | SER | A | 223 | 32.418 | 5.007 | 10.896 | 1.00 | 44.42 | C |
| ATOM | 1711 | CB | SER | A | 223 | 32.406 | 4.941 | 12.420 | 1.00 | 46.33 | C |
| ATOM | 1712 | OG | SER | A | 223 | 32.483 | 6.252 | 12.970 | 1.00 | 67.62 | O |
| ATOM | 1713 | C | SER | A | 223 | 33.208 | 3.827 | 10.336 | 1.00 | 45.39 | C |
| ATOM | 1714 | O | SER | A | 223 | 34.435 | 3.848 | 10.237 | 1.00 | 45.27 | O |
| ATOM | 1715 | N | THR | A | 224 | 32.495 | 2.750 | 10.043 | 1.00 | 53.19 | N |
| ATOM | 1716 | CA | THR | A | 224 | 33.127 | 1.525 | 9.556 | 1.00 | 54.34 | C |
| ATOM | 1717 | CB | THR | A | 224 | 33.254 | 0.422 | 10.681 | 1.00 | 59.06 | C |
| ATOM | 1718 | OG1 | THR | A | 224 | 32.030 | 0.310 | 11.438 | 1.00 | 77.63 | O |
| ATOM | 1719 | CG2 | THR | A | 224 | 34.311 | 0.806 | 11.744 | 1.00 | 53.15 | C |
| ATOM | 1720 | C | THR | A | 224 | 32.330 | 0.997 | 8.358 | 1.00 | 47.23 | C |
| ATOM | 1721 | O | THR | A | 224 | 31.189 | 1.420 | 8.101 | 1.00 | 28.65 | O |
| ATOM | 1722 | N | VAL | A | 225 | 32.979 | 0.134 | 7.583 | 1.00 | 43.38 | N |
| ATOM | 1723 | CA | VAL | A | 225 | 32.393 | −0.348 | 6.345 | 1.00 | 35.37 | C |
| ATOM | 1724 | CB | VAL | A | 225 | 33.080 | 0.238 | 5.076 | 1.00 | 37.78 | C |
| ATOM | 1725 | CG1 | VAL | A | 225 | 34.590 | 0.183 | 5.169 | 1.00 | 29.85 | C |
| ATOM | 1726 | CG2 | VAL | A | 225 | 32.634 | −0.500 | 3.838 | 1.00 | 19.96 | C |
| ATOM | 1727 | C | VAL | A | 225 | 32.592 | −1.835 | 6.355 | 1.00 | 44.28 | C |
| ATOM | 1728 | O | VAL | A | 225 | 33.609 | −2.321 | 6.859 | 1.00 | 47.10 | O |
| ATOM | 1729 | N | THR | A | 226 | 31.594 | −2.564 | 5.865 | 1.00 | 46.74 | N |
| ATOM | 1730 | CA | THR | A | 226 | 31.717 | −4.014 | 5.785 | 1.00 | 44.72 | C |
| ATOM | 1731 | CB | THR | A | 226 | 30.513 | −4.664 | 6.456 | 1.00 | 41.22 | C |
| ATOM | 1732 | OG1 | THR | A | 226 | 29.369 | −3.829 | 6.249 | 1.00 | 62.95 | O |
| ATOM | 1733 | CG2 | THR | A | 226 | 30.671 | −4.611 | 7.967 | 1.00 | 41.62 | C |
| ATOM | 1734 | C | THR | A | 226 | 31.771 | −4.360 | 4.316 | 1.00 | 40.61 | C |
| ATOM | 1735 | O | THR | A | 226 | 30.966 | −3.853 | 3.544 | 1.00 | 51.72 | O |
| ATOM | 1736 | N | LEU | A | 227 | 32.767 | −5.140 | 3.916 | 1.00 | 30.12 | N |
| ATOM | 1737 | CA | LEU | A | 227 | 32.790 | −5.720 | 2.575 | 1.00 | 33.76 | C |
| ATOM | 1738 | CB | LEU | A | 227 | 34.126 | −5.415 | 1.894 | 1.00 | 41.86 | C |
| ATOM | 1739 | CG | LEU | A | 227 | 35.077 | −4.481 | 2.634 | 1.00 | 37.66 | C |
| ATOM | 1740 | CD1 | LEU | A | 227 | 36.515 | −4.886 | 2.326 | 1.00 | 38.05 | C |
| ATOM | 1741 | CD2 | LEU | A | 227 | 34.804 | −3.060 | 2.196 | 1.00 | 57.18 | C |
| ATOM | 1742 | C | LEU | A | 227 | 32.564 | −7.230 | 2.562 | 1.00 | 29.05 | C |
| ATOM | 1743 | O | LEU | A | 227 | 33.424 | −7.999 | 2.964 | 1.00 | 32.68 | O |
| ATOM | 1744 | N | SER | A | 228 | 31.445 | −7.676 | 2.021 | 1.00 | 35.16 | N |
| ATOM | 1745 | CA | SER | A | 228 | 31.211 | −9.113 | 1.939 | 1.00 | 43.32 | C |
| ATOM | 1746 | CB | SER | A | 228 | 29.817 | −9.475 | 2.447 | 1.00 | 37.86 | C |
| ATOM | 1747 | OG | SER | A | 228 | 29.479 | −8.685 | 3.583 | 1.00 | 57.36 | O |
| ATOM | 1748 | C | SER | A | 228 | 31.372 | −9.589 | 0.506 | 1.00 | 51.00 | C |
| ATOM | 1749 | O | SER | A | 228 | 30.786 | −9.025 | −0.428 | 1.00 | 54.92 | O |
| ATOM | 1750 | N | MET | A | 229 | 32.160 | −10.647 | 0.351 | 1.00 | 54.99 | N |
| ATOM | 1751 | CA | MET | A | 229 | 32.493 | −11.190 | −0.963 | 1.00 | 52.79 | C |
| ATOM | 1752 | CB | MET | A | 229 | 33.797 | −10.570 | −1.485 | 1.00 | 51.70 | C |
| ATOM | 1753 | CG | MET | A | 229 | 34.771 | −10.118 | −0.397 | 1.00 | 47.71 | C |
| ATOM | 1754 | SD | MET | A | 229 | 36.490 | −10.435 | −0.865 | 1.00 | 62.28 | S |
| ATOM | 1755 | CE | MET | A | 229 | 37.330 | −10.234 | 0.741 | 1.00 | 42.71 | C |
| ATOM | 1756 | C | MET | A | 229 | 32.585 | −12.724 | −0.949 | 1.00 | 55.14 | C |
| ATOM | 1757 | O | MET | A | 229 | 32.880 | −13.351 | 0.068 | 1.00 | 53.95 | O |
| ATOM | 1758 | N | SER | A | 230 | 32.332 | −13.322 | −2.104 | 1.00 | 53.84 | N |
| ATOM | 1759 | CA | SER | A | 230 | 32.243 | −14.770 | −2.203 | 1.00 | 46.89 | C |
| ATOM | 1760 | CB | SER | A | 230 | 30.815 | −15.190 | −1.852 | 1.00 | 29.44 | C |
| ATOM | 1761 | OG | SER | A | 230 | 30.608 | −16.507 | −2.318 | 1.00 | 54.32 | O |
| ATOM | 1762 | C | SER | A | 230 | 32.554 | −15.143 | −3.656 | 1.00 | 48.03 | C |
| ATOM | 1763 | O | SER | A | 230 | 32.339 | −14.351 | −4.569 | 1.00 | 56.50 | O |
| ATOM | 1764 | N | ALA | A | 231 | 33.110 | −16.322 | −3.900 | 1.00 | 47.98 | N |
| ATOM | 1765 | CA | ALA | A | 231 | 33.608 | −16.594 | −5.247 | 1.00 | 44.56 | C |
| ATOM | 1766 | CB | ALA | A | 231 | 34.322 | −17.907 | −5.271 | 1.00 | 37.89 | C |
| ATOM | 1767 | C | ALA | A | 231 | 32.476 | −16.561 | −6.271 | 1.00 | 40.23 | C |
| ATOM | 1768 | O | ALA | A | 231 | 31.409 | −17.130 | −6.040 | 1.00 | 43.54 | O |
| ATOM | 1769 | N | ASP | A | 232 | 32.697 | −15.862 | −7.378 | 1.00 | 40.27 | N |
| ATOM | 1770 | CA | ASP | A | 232 | 31.782 | −15.903 | −8.524 | 1.00 | 54.07 | C |
| ATOM | 1771 | CB | ASP | A | 232 | 31.406 | −17.345 | −8.899 | 1.00 | 67.02 | C |
| ATOM | 1772 | CG | ASP | A | 232 | 32.477 | −18.049 | −9.725 | 1.00 | 74.43 | C |
| ATOM | 1773 | OD1 | ASP | A | 232 | 33.343 | −18.740 | −9.135 | 1.00 | 67.17 | O |
| ATOM | 1774 | OD2 | ASP | A | 232 | 32.466 | −18.052 | −10.975 | 1.00 | 67.85 | O |
| ATOM | 1775 | C | ASP | A | 232 | 30.485 | −15.132 | −8.289 | 1.00 | 49.90 | C |
| ATOM | 1776 | O | ASP | A | 232 | 29.545 | −15.229 | −9.085 | 1.00 | 41.18 | O |
| ATOM | 1777 | N | VAL | A | 233 | 30.392 | −14.455 | −7.149 | 1.00 | 48.84 | N |
| ATOM | 1778 | CA | VAL | A | 233 | 29.332 | −13.460 | −6.947 | 1.00 | 47.15 | C |
| ATOM | 1779 | CB | VAL | A | 233 | 28.440 | −13.799 | −5.728 | 1.00 | 45.26 | C |
| ATOM | 1780 | CG1 | VAL | A | 233 | 28.456 | −15.321 | −5.471 | 1.00 | 48.90 | C |
| ATOM | 1781 | CG2 | VAL | A | 233 | 28.933 | −13.026 | −4.479 | 1.00 | 56.07 | C |
| ATOM | 1782 | C | VAL | A | 233 | 29.912 | −12.052 | −6.769 | 1.00 | 43.81 | C |
| ATOM | 1783 | O | VAL | A | 233 | 31.074 | −11.853 | −6.403 | 1.00 | 49.20 | O |
| ATOM | 1784 | N | PRO | A | 234 | 29.077 | −11.056 | −7.009 | 1.00 | 40.14 | N |
| ATOM | 1785 | CA | PRO | A | 234 | 29.493 | −9.664 | −6.823 | 1.00 | 31.41 | C |
| ATOM | 1786 | CB | PRO | A | 234 | 28.289 | −8.869 | −7.380 | 1.00 | 16.02 | C |
| ATOM | 1787 | CG | PRO | A | 234 | 27.131 | −9.789 | −7.320 | 1.00 | 10.53 | C |
| ATOM | 1788 | CD | PRO | A | 234 | 27.694 | −11.174 | −7.518 | 1.00 | 44.90 | C |
| ATOM | 1789 | C | PRO | A | 234 | 29.870 | −9.317 | −5.360 | 1.00 | 27.03 | C |
| ATOM | 1790 | O | PRO | A | 234 | 29.301 | −9.841 | −4.404 | 1.00 | 29.63 | O |
| ATOM | 1791 | N | LEU | A | 235 | 30.837 | −8.428 | −5.200 | 1.00 | 23.81 | N |
| ATOM | 1792 | CA | LEU | A | 235 | 31.182 | −7.901 | −3.899 | 1.00 | 22.41 | C |
| ATOM | 1793 | CB | LEU | A | 235 | 32.581 | −7.307 | −3.960 | 1.00 | 4.53 | C |
| ATOM | 1794 | CG | LEU | A | 235 | 32.870 | −6.171 | −3.003 | 1.00 | 9.85 | C |
| ATOM | 1795 | CD1 | LEU | A | 235 | 33.264 | −6.701 | −1.642 | 1.00 | 23.13 | C |
| ATOM | 1796 | CD2 | LEU | A | 235 | 33.996 | −5.377 | −3.585 | 1.00 | 20.75 | C |
| ATOM | 1797 | C | LEU | A | 235 | 30.206 | −6.847 | −3.408 | 1.00 | 30.46 | C |
| ATOM | 1798 | O | LEU | A | 235 | 29.761 | −6.009 | −4.185 | 1.00 | 33.03 | O |
| ATOM | 1799 | N | VAL | A | 236 | 29.930 | −6.865 | −2.102 | 1.00 | 34.01 | N |
| ATOM | 1800 | CA | VAL | A | 236 | 29.017 | −5.913 | −1.490 | 1.00 | 35.52 | C |
| ATOM | 1801 | CB | VAL | A | 236 | 27.836 | −6.615 | −0.773 | 1.00 | 39.65 | C |
| ATOM | 1802 | CG1 | VAL | A | 236 | 26.618 | −5.694 | −0.681 | 1.00 | 38.82 | C |
| ATOM | 1803 | CG2 | VAL | A | 236 | 27.460 | −7.902 | −1.499 | 1.00 | 52.55 | C |
| ATOM | 1804 | C | VAL | A | 236 | 29.785 | −5.050 | −0.504 | 1.00 | 35.47 | C |
| ATOM | 1805 | O | VAL | A | 236 | 30.401 | −5.533 | 0.436 | 1.00 | 29.93 | O |
| ATOM | 1806 | N | VAL | A | 237 | 29.717 | −3.749 | −0.736 | 1.00 | 41.74 | N |
| ATOM | 1807 | CA | VAL | A | 237 | 30.208 | −2.753 | 0.206 | 1.00 | 38.66 | C |
| ATOM | 1808 | CB | VAL | A | 237 | 31.069 | −1.681 | −0.512 | 1.00 | 39.24 | C |
| ATOM | 1809 | CG1 | VAL | A | 237 | 32.016 | −1.034 | 0.463 | 1.00 | 38.72 | C |
| ATOM | 1810 | CG2 | VAL | A | 237 | 31.867 | −2.294 | −1.640 | 1.00 | 40.32 | C |
| ATOM | 1811 | C | VAL | A | 237 | 29.036 | −2.060 | 0.896 | 1.00 | 39.45 | C |
| ATOM | 1812 | O | VAL | A | 237 | 28.202 | −1.415 | 0.249 | 1.00 | 38.90 | O |
| ATOM | 1813 | N | GLU | A | 238 | 28.993 | −2.181 | 2.219 | 1.00 | 38.08 | N |
| ATOM | 1814 | CA | GLU | A | 238 | 27.846 | −1.720 | 2.988 | 1.00 | 40.06 | C |
| ATOM | 1815 | CB | GLU | A | 238 | 27.219 | −2.868 | 3.774 | 1.00 | 45.78 | C |
| ATOM | 1816 | CG | GLU | A | 238 | 25.956 | −2.466 | 4.521 | 1.00 | 61.75 | C |
| ATOM | 1817 | CD | GLU | A | 238 | 25.220 | −3.651 | 5.129 | 1.00 | 79.55 | C |
| ATOM | 1818 | OE1 | GLU | A | 238 | 25.900 | −4.657 | 5.462 | 1.00 | 88.39 | O |
| ATOM | 1819 | OE2 | GLU | A | 238 | 23.972 | −3.555 | 5.309 | 1.00 | 64.51 | O |
| ATOM | 1820 | C | GLU | A | 238 | 28.286 | −0.653 | 3.960 | 1.00 | 35.04 | C |
| ATOM | 1821 | O | GLU | A | 238 | 29.112 | −0.903 | 4.829 | 1.00 | 34.39 | O |
| ATOM | 1822 | N | TYR | A | 239 | 27.719 | 0.535 | 3.789 | 1.00 | 41.08 | N |
| ATOM | 1823 | CA | TYR | A | 239 | 27.854 | 1.629 | 4.746 | 1.00 | 40.84 | C |
| ATOM | 1824 | CB | TYR | A | 239 | 28.164 | 2.921 | 4.002 | 1.00 | 41.81 | C |
| ATOM | 1825 | CG | TYR | A | 239 | 29.484 | 2.911 | 3.280 | 1.00 | 37.57 | C |
| ATOM | 1826 | CD1 | TYR | A | 239 | 29.555 | 2.603 | 1.924 | 1.00 | 36.41 | C |
| ATOM | 1827 | CE1 | TYR | A | 239 | 30.762 | 2.594 | 1.265 | 1.00 | 37.60 | C |
| ATOM | 1828 | CZ | TYR | A | 239 | 31.911 | 2.940 | 1.954 | 1.00 | 24.43 | C |
| ATOM | 1829 | OH | TYR | A | 239 | 33.130 | 2.929 | 1.318 | 1.00 | 41.67 | O |
| ATOM | 1830 | CE2 | TYR | A | 239 | 31.857 | 3.290 | 3.284 | 1.00 | 10.16 | C |
| ATOM | 1831 | CD2 | TYR | A | 239 | 30.652 | 3.250 | 3.945 | 1.00 | 34.46 | C |
| ATOM | 1832 | C | TYR | A | 239 | 26.541 | 1.825 | 5.500 | 1.00 | 44.46 | C |
| ATOM | 1833 | O | TYR | A | 239 | 25.552 | 2.293 | 4.906 | 1.00 | 29.74 | O |
| ATOM | 1834 | N | LYS | A | 240 | 26.566 | 1.515 | 6.806 | 1.00 | 52.96 | N |
| ATOM | 1835 | CA | LYS | A | 240 | 25.361 | 1.458 | 7.656 | 1.00 | 56.99 | C |
| ATOM | 1836 | CB | LYS | A | 240 | 25.537 | 0.454 | 8.814 | 1.00 | 58.40 | C |
| ATOM | 1837 | CG | LYS | A | 240 | 25.537 | −1.029 | 8.410 | 1.00 | 61.28 | C |
| ATOM | 1838 | CD | LYS | A | 240 | 24.400 | −1.835 | 9.052 | 1.00 | 46.54 | C |
| ATOM | 1839 | CE | LYS | A | 240 | 24.889 | −2.637 | 10.263 | 1.00 | 66.45 | C |
| ATOM | 1840 | NZ | LYS | A | 240 | 24.154 | −3.915 | 10.501 | 1.00 | 66.58 | N |
| ATOM | 1841 | C | LYS | A | 240 | 24.989 | 2.841 | 8.215 | 1.00 | 50.10 | C |
| ATOM | 1842 | O | LYS | A | 240 | 25.799 | 3.480 | 8.909 | 1.00 | 49.17 | O |
| ATOM | 1843 | N | ILE | A | 241 | 23.799 | 3.327 | 7.856 | 1.00 | 37.68 | N |
| ATOM | 1844 | CA | ILE | A | 241 | 23.398 | 4.652 | 8.290 | 1.00 | 40.47 | C |
| ATOM | 1845 | CB | ILE | A | 241 | 22.353 | 5.225 | 7.354 | 1.00 | 41.89 | C |
| ATOM | 1846 | CG1 | ILE | A | 241 | 22.868 | 5.196 | 5.922 | 1.00 | 42.51 | C |
| ATOM | 1847 | CD1 | ILE | A | 241 | 21.861 | 5.679 | 4.888 | 1.00 | 22.00 | C |
| ATOM | 1848 | CG2 | ILE | A | 241 | 22.011 | 6.637 | 7.777 | 1.00 | 47.27 | C |
| ATOM | 1849 | C | ILE | A | 241 | 22.740 | 4.519 | 9.635 | 1.00 | 47.39 | C |
| ATOM | 1850 | O | ILE | A | 241 | 21.584 | 4.089 | 9.714 | 1.00 | 36.99 | O |
| ATOM | 1851 | N | ALA | A | 242 | 23.485 | 4.834 | 10.693 | 1.00 | 49.96 | N |
| ATOM | 1852 | CA | ALA | A | 242 | 23.089 | 4.381 | 12.020 | 1.00 | 47.95 | C |
| ATOM | 1853 | CB | ALA | A | 242 | 24.083 | 4.865 | 13.093 | 1.00 | 41.32 | C |
| ATOM | 1854 | C | ALA | A | 242 | 21.673 | 4.889 | 12.284 | 1.00 | 44.52 | C |
| ATOM | 1855 | O | ALA | A | 242 | 21.390 | 6.053 | 12.034 | 1.00 | 36.06 | O |
| ATOM | 1856 | N | ASP | A | 243 | 20.795 | 3.994 | 12.742 | 1.00 | 52.10 | N |
| ATOM | 1857 | CA | ASP | A | 243 | 19.495 | 4.334 | 13.332 | 1.00 | 52.16 | C |
| ATOM | 1858 | CB | ASP | A | 243 | 19.612 | 5.405 | 14.431 | 1.00 | 54.74 | C |
| ATOM | 1859 | CG | ASP | A | 243 | 18.335 | 5.524 | 15.262 | 1.00 | 66.17 | C |
| ATOM | 1860 | OD1 | ASP | A | 243 | 17.643 | 6.563 | 15.143 | 1.00 | 81.42 | O |
| ATOM | 1861 | OD2 | ASP | A | 243 | 17.908 | 4.616 | 16.025 | 1.00 | 76.40 | O |
| ATOM | 1862 | C | ASP | A | 243 | 18.556 | 4.801 | 12.240 | 1.00 | 52.32 | C |
| ATOM | 1863 | O | ASP | A | 243 | 17.686 | 5.646 | 12.475 | 1.00 | 58.46 | O |
| ATOM | 1864 | N | MET | A | 244 | 18.826 | 4.362 | 11.017 | 1.00 | 46.47 | N |
| ATOM | 1865 | CA | MET | A | 244 | 18.106 | 4.903 | 9.867 | 1.00 | 43.77 | C |
| ATOM | 1866 | CB | MET | A | 244 | 18.862 | 6.113 | 9.261 | 1.00 | 50.34 | C |
| ATOM | 1867 | CG | MET | A | 244 | 18.294 | 7.515 | 9.560 | 1.00 | 34.71 | C |
| ATOM | 1868 | SD | MET | A | 244 | 16.578 | 7.605 | 9.118 | 1.00 | 64.00 | S |
| ATOM | 1869 | CE | MET | A | 244 | 16.647 | 8.358 | 7.526 | 1.00 | 69.65 | C |
| ATOM | 1870 | C | MET | A | 244 | 17.962 | 3.769 | 8.848 | 1.00 | 45.02 | C |
| ATOM | 1871 | O | MET | A | 244 | 16.884 | 3.557 | 8.281 | 1.00 | 25.47 | O |
| ATOM | 1872 | N | GLY | A | 245 | 19.066 | 3.056 | 8.611 | 1.00 | 47.77 | N |
| ATOM | 1873 | CA | GLY | A | 245 | 19.173 | 2.158 | 7.455 | 1.00 | 38.98 | C |
| ATOM | 1874 | C | GLY | A | 245 | 20.611 | 1.872 | 7.070 | 1.00 | 38.23 | C |
| ATOM | 1875 | O | GLY | A | 245 | 21.524 | 1.907 | 7.913 | 1.00 | 38.54 | O |
| ATOM | 1876 | N | HIS | A | 246 | 20.810 | 1.565 | 5.789 | 1.00 | 35.66 | N |
| ATOM | 1877 | CA | HIS | A | 246 | 22.130 | 1.158 | 5.291 | 1.00 | 39.81 | C |
| ATOM | 1878 | CB | HIS | A | 246 | 22.244 | −0.365 | 5.284 | 1.00 | 28.46 | C |
| ATOM | 1879 | CG | HIS | A | 246 | 21.262 | −1.014 | 4.360 | 1.00 | 42.05 | C |
| ATOM | 1880 | ND1 | HIS | A | 246 | 19.936 | −1.211 | 4.697 | 1.00 | 67.73 | N |
| ATOM | 1881 | CE1 | HIS | A | 246 | 19.299 | −1.771 | 3.678 | 1.00 | 76.99 | C |
| ATOM | 1882 | NE2 | HIS | A | 246 | 20.150 | −1.901 | 2.673 | 1.00 | 59.79 | N |
| ATOM | 1883 | CD2 | HIS | A | 246 | 21.370 | −1.390 | 3.057 | 1.00 | 65.98 | C |
| ATOM | 1884 | C | HIS | A | 246 | 22.317 | 1.656 | 3.864 | 1.00 | 43.37 | C |
| ATOM | 1885 | O | HIS | A | 246 | 21.355 | 1.964 | 3.166 | 1.00 | 53.00 | O |
| ATOM | 1886 | N | LEU | A | 247 | 23.550 | 1.659 | 3.388 | 1.00 | 41.33 | N |
| ATOM | 1887 | CA | LEU | A | 247 | 23.752 | 2.005 | 2.005 | 1.00 | 30.36 | C |
| ATOM | 1888 | CB | LEU | A | 247 | 24.385 | 3.381 | 1.935 | 1.00 | 34.56 | C |
| ATOM | 1889 | CG | LEU | A | 247 | 24.421 | 4.081 | 0.567 | 1.00 | 51.04 | C |
| ATOM | 1890 | CD1 | LEU | A | 247 | 23.108 | 3.970 | −0.209 | 1.00 | 41.10 | C |
| ATOM | 1891 | CD2 | LEU | A | 247 | 24.856 | 5.544 | 0.732 | 1.00 | 28.92 | C |
| ATOM | 1892 | C | LEU | A | 247 | 24.676 | 0.962 | 1.429 | 1.00 | 29.87 | C |
| ATOM | 1893 | O | LEU | A | 247 | 25.877 | 0.994 | 1.686 | 1.00 | 33.37 | O |
| ATOM | 1894 | N | LYS | A | 248 | 24.108 | 0.046 | 0.647 | 1.00 | 30.96 | N |
| ATOM | 1895 | CA | LYS | A | 248 | 24.848 | −1.061 | 0.044 | 1.00 | 33.44 | C |
| ATOM | 1896 | CB | LYS | A | 248 | 23.962 | −2.296 | 0.059 | 1.00 | 38.48 | C |
| ATOM | 1897 | CG | LYS | A | 248 | 24.265 | −3.285 | 1.181 | 1.00 | 53.28 | C |
| ATOM | 1898 | CD | LYS | A | 248 | 23.340 | −4.496 | 1.067 | 1.00 | 55.20 | C |
| ATOM | 1899 | CE | LYS | A | 248 | 22.816 | −4.919 | 2.425 | 1.00 | 54.73 | C |
| ATOM | 1900 | NZ | LYS | A | 248 | 21.375 | −5.297 | 2.375 | 1.00 | 64.46 | N |
| ATOM | 1901 | C | LYS | A | 248 | 25.301 | −0.799 | −1.406 | 1.00 | 34.80 | C |
| ATOM | 1902 | O | LYS | A | 248 | 24.618 | −0.123 | −2.184 | 1.00 | 31.64 | O |
| ATOM | 1903 | N | TYR | A | 249 | 26.470 | −1.331 | −1.753 | 1.00 | 33.22 | N |
| ATOM | 1904 | CA | TYR | A | 249 | 27.104 | −1.049 | −3.029 | 1.00 | 28.63 | C |
| ATOM | 1905 | CB | TYR | A | 249 | 28.324 | −0.153 | −2.775 | 1.00 | 22.32 | C |
| ATOM | 1906 | CG | TYR | A | 249 | 28.026 | 1.325 | −2.710 | 1.00 | 17.81 | C |
| ATOM | 1907 | CD1 | TYR | A | 249 | 27.516 | 1.997 | −3.805 | 1.00 | 32.70 | C |
| ATOM | 1908 | CE1 | TYR | A | 249 | 27.199 | 3.329 | −3.725 | 1.00 | 39.26 | C |
| ATOM | 1909 | CZ | TYR | A | 249 | 27.441 | 4.021 | −2.557 | 1.00 | 58.99 | C |
| ATOM | 1910 | OH | TYR | A | 249 | 27.093 | 5.353 | −2.463 | 1.00 | 69.15 | O |
| ATOM | 1911 | CE2 | TYR | A | 249 | 28.028 | 3.391 | −1.491 | 1.00 | 46.42 | C |
| ATOM | 1912 | CD2 | TYR | A | 249 | 28.306 | 2.054 | −1.570 | 1.00 | 39.72 | C |
| ATOM | 1913 | C | TYR | A | 249 | 27.574 | −2.383 | −3.628 | 1.00 | 34.73 | C |
| ATOM | 1914 | O | TYR | A | 249 | 28.617 | −2.921 | −3.241 | 1.00 | 37.42 | O |
| ATOM | 1915 | N | TYR | A | 250 | 26.841 | −2.923 | −4.593 | 1.00 | 31.60 | N |
| ATOM | 1916 | CA | TYR | A | 250 | 27.285 | −4.181 | −5.201 | 1.00 | 32.79 | C |
| ATOM | 1917 | CB | TYR | A | 250 | 26.103 | −4.984 | −5.712 | 1.00 | 37.48 | C |
| ATOM | 1918 | CG | TYR | A | 250 | 25.106 | −5.372 | −4.638 | 1.00 | 53.66 | C |
| ATOM | 1919 | CD1 | TYR | A | 250 | 24.163 | −4.457 | −4.171 | 1.00 | 45.07 | C |
| ATOM | 1920 | CE1 | TYR | A | 250 | 23.233 | −4.808 | −3.207 | 1.00 | 32.95 | C |
| ATOM | 1921 | CZ | TYR | A | 250 | 23.196 | −6.082 | −2.722 | 1.00 | 47.61 | C |
| ATOM | 1922 | OH | TYR | A | 250 | 22.217 | −6.368 | −1.798 | 1.00 | 60.53 | O |
| ATOM | 1923 | CE2 | TYR | A | 250 | 24.106 | −7.026 | −3.173 | 1.00 | 65.69 | C |
| ATOM | 1924 | CD2 | TYR | A | 250 | 25.061 | −6.667 | −4.129 | 1.00 | 64.57 | C |
| ATOM | 1925 | C | TYR | A | 250 | 28.215 | −3.929 | −6.364 | 1.00 | 35.17 | C |
| ATOM | 1926 | O | TYR | A | 250 | 28.065 | −2.934 | −7.081 | 1.00 | 37.57 | O |
| ATOM | 1927 | N | LEU | A | 251 | 29.161 | −4.840 | −6.566 | 1.00 | 32.40 | N |
| ATOM | 1928 | CA | LEU | A | 251 | 30.160 | −4.658 | −7.612 | 1.00 | 31.75 | C |
| ATOM | 1929 | CB | LEU | A | 251 | 31.419 | −3.976 | −7.087 | 1.00 | 35.38 | C |
| ATOM | 1930 | CG | LEU | A | 251 | 32.489 | −3.637 | −8.137 | 1.00 | 36.11 | C |
| ATOM | 1931 | CD1 | LEU | A | 251 | 31.853 | −2.861 | −9.281 | 1.00 | 22.01 | C |
| ATOM | 1932 | CD2 | LEU | A | 251 | 33.640 | −2.825 | −7.502 | 1.00 | 26.08 | C |
| ATOM | 1933 | C | LEU | A | 251 | 30.585 | −5.968 | −8.177 | 1.00 | 30.87 | C |
| ATOM | 1934 | O | LEU | A | 251 | 31.049 | −6.836 | −7.452 | 1.00 | 36.16 | O |
| ATOM | 1935 | N | ALA | A | 252 | 30.592 | −6.053 | −9.493 | 1.00 | 34.62 | N |
| ATOM | 1936 | CA | ALA | A | 252 | 30.957 | −7.296 | −10.146 | 1.00 | 35.82 | C |
| ATOM | 1937 | CB | ALA | A | 252 | 30.456 | −7.279 | −11.563 | 1.00 | 48.99 | C |
| ATOM | 1938 | C | ALA | A | 252 | 32.462 | −7.470 | −10.143 | 1.00 | 37.14 | C |
| ATOM | 1939 | O | ALA | A | 252 | 33.212 | −6.515 | −10.346 | 1.00 | 47.30 | O |
| ATOM | 1940 | N | PRO | A | 253 | 32.890 | −8.719 | −10.050 | 1.00 | 34.67 | N |
| ATOM | 1941 | CA | PRO | A | 253 | 34.278 | −9.111 | −10.329 | 1.00 | 31.02 | C |
| ATOM | 1942 | CB | PRO | A | 253 | 34.265 | −10.605 | −10.044 | 1.00 | 27.29 | C |
| ATOM | 1943 | CG | PRO | A | 253 | 33.079 | −10.808 | −9.177 | 1.00 | 33.35 | C |
| ATOM | 1944 | CD | PRO | A | 253 | 32.047 | −9.856 | −9.670 | 1.00 | 34.40 | C |
| ATOM | 1945 | C | PRO | A | 253 | 34.696 | −8.879 | −11.780 | 1.00 | 34.56 | C |
| ATOM | 1946 | O | PRO | A | 253 | 33.885 | −8.945 | −12.699 | 1.00 | 47.64 | O |
| ATOM | 1947 | N | LYS | A | 254 | 35.976 | −8.631 | −11.994 | 1.00 | 31.55 | N |
| ATOM | 1948 | CA | LYS | A | 254 | 36.499 | −8.482 | −13.337 | 1.00 | 36.31 | C |
| ATOM | 1949 | CB | LYS | A | 254 | 37.823 | −7.729 | −13.310 | 1.00 | 26.90 | C |
| ATOM | 1950 | CG | LYS | A | 254 | 37.761 | −6.403 | −14.064 | 1.00 | 50.19 | C |
| ATOM | 1951 | CD | LYS | A | 254 | 38.842 | −5.432 | −13.605 | 1.00 | 33.09 | C |
| ATOM | 1952 | CE | LYS | A | 254 | 39.574 | −4.783 | −14.766 | 1.00 | 50.41 | C |
| ATOM | 1953 | NZ | LYS | A | 254 | 39.601 | −3.291 | −14.606 | 1.00 | 65.27 | N |
| ATOM | 1954 | C | LYS | A | 254 | 36.664 | −9.826 | −14.043 | 1.00 | 53.39 | C |
| ATOM | 1955 | O | LYS | A | 254 | 36.736 | −10.884 | −13.408 | 1.00 | 56.79 | O |
| ATOM | 1956 | N | ILE | A | 255 | 36.688 | −9.770 | −15.375 | 1.00 | 64.66 | N |
| ATOM | 1957 | CA | ILE | A | 255 | 36.841 | −10.947 | −16.233 | 1.00 | 59.67 | C |
| ATOM | 1958 | CB | ILE | A | 255 | 38.312 | −11.409 | −16.287 | 1.00 | 60.16 | C |
| ATOM | 1959 | CG1 | ILE | A | 255 | 39.264 | −10.203 | −16.388 | 1.00 | 70.99 | C |
| ATOM | 1960 | CD1 | ILE | A | 255 | 39.381 | −9.533 | −17.777 | 1.00 | 65.00 | C |
| ATOM | 1961 | CG2 | ILE | A | 255 | 38.514 | −12.366 | −17.454 | 1.00 | 77.94 | C |
| ATOM | 1962 | C | ILE | A | 255 | 35.921 | −12.093 | −15.852 | 1.00 | 49.93 | C |
| ATOM | 1963 | O | ILE | A | 255 | 36.353 | −13.016 | −15.179 | 1.00 | 59.84 | O |
| ATOM | 1964 | N | MET | B | 1 | 30.958 | 29.281 | 36.055 | 1.00 | 54.73 | N |
| ATOM | 1965 | CA | MET | B | 1 | 30.625 | 30.703 | 36.354 | 1.00 | 61.07 | C |
| ATOM | 1966 | CB | MET | B | 1 | 29.351 | 30.778 | 37.217 | 1.00 | 63.12 | C |
| ATOM | 1967 | CG | MET | B | 1 | 28.622 | 32.130 | 37.226 | 1.00 | 69.64 | C |
| ATOM | 1968 | SD | MET | B | 1 | 28.344 | 32.863 | 38.881 | 1.00 | 98.75 | S |
| ATOM | 1969 | CE | MET | B | 1 | 26.824 | 32.027 | 39.428 | 1.00 | 85.27 | C |
| ATOM | 1970 | C | MET | B | 1 | 31.803 | 31.409 | 37.052 | 1.00 | 62.47 | C |
| ATOM | 1971 | O | MET | B | 1 | 32.368 | 30.900 | 38.046 | 1.00 | 60.05 | O |
| ATOM | 1972 | N | PHE | B | 2 | 32.130 | 32.606 | 36.547 | 1.00 | 58.52 | N |
| ATOM | 1973 | CA | PHE | B | 2 | 33.167 | 33.470 | 37.132 | 1.00 | 53.89 | C |
| ATOM | 1974 | CB | PHE | B | 2 | 34.114 | 33.999 | 36.038 | 1.00 | 55.74 | C |
| ATOM | 1975 | CG | PHE | B | 2 | 34.898 | 35.240 | 36.435 | 1.00 | 64.79 | C |
| ATOM | 1976 | CD1 | PHE | B | 2 | 35.625 | 35.276 | 37.640 | 1.00 | 52.55 | C |
| ATOM | 1977 | CE1 | PHE | B | 2 | 36.383 | 36.403 | 37.983 | 1.00 | 39.16 | C |
| ATOM | 1978 | CZ | PHE | B | 2 | 36.391 | 37.517 | 37.135 | 1.00 | 30.71 | C |
| ATOM | 1979 | CE2 | PHE | B | 2 | 35.672 | 37.490 | 35.911 | 1.00 | 47.07 | C |
| ATOM | 1980 | CD2 | PHE | B | 2 | 34.942 | 36.358 | 35.570 | 1.00 | 46.88 | C |
| ATOM | 1981 | C | PHE | B | 2 | 32.582 | 34.627 | 37.938 | 1.00 | 43.13 | C |
| ATOM | 1982 | O | PHE | B | 2 | 31.633 | 35.274 | 37.501 | 1.00 | 42.33 | O |
| ATOM | 1983 | N | GLU | B | 3 | 33.107 | 34.860 | 39.134 | 1.00 | 37.35 | N |
| ATOM | 1984 | CA | GLU | B | 3 | 32.521 | 35.889 | 39.992 | 1.00 | 46.93 | C |
| ATOM | 1985 | CB | GLU | B | 3 | 31.272 | 35.377 | 40.740 | 1.00 | 42.57 | C |
| ATOM | 1986 | CG | GLU | B | 3 | 30.668 | 36.378 | 41.738 | 1.00 | 59.42 | C |
| ATOM | 1987 | CD | GLU | B | 3 | 29.162 | 36.207 | 41.937 | 1.00 | 79.64 | C |
| ATOM | 1988 | OE1 | GLU | B | 3 | 28.550 | 35.377 | 41.222 | 1.00 | 93.05 | O |
| ATOM | 1989 | OE2 | GLU | B | 3 | 28.588 | 36.920 | 42.804 | 1.00 | 66.10 | O |
| ATOM | 1990 | C | GLU | B | 3 | 33.553 | 36.489 | 40.952 | 1.00 | 48.54 | C |
| ATOM | 1991 | O | GLU | B | 3 | 33.978 | 35.841 | 41.910 | 1.00 | 51.98 | O |
| ATOM | 1992 | N | ALA | B | 4 | 33.971 | 37.722 | 40.676 | 1.00 | 40.22 | N |
| ATOM | 1993 | CA | ALA | B | 4 | 34.997 | 38.356 | 41.476 | 1.00 | 35.64 | C |
| ATOM | 1994 | CB | ALA | B | 4 | 36.210 | 38.627 | 40.643 | 1.00 | 44.20 | C |
| ATOM | 1995 | C | ALA | B | 4 | 34.457 | 39.638 | 42.040 | 1.00 | 34.55 | C |
| ATOM | 1996 | O | ALA | B | 4 | 34.001 | 40.502 | 41.312 | 1.00 | 46.88 | O |
| ATOM | 1997 | N | ARG | B | 5 | 34.468 | 39.753 | 43.353 | 1.00 | 32.06 | N |
| ATOM | 1998 | CA | ARG | B | 5 | 33.921 | 40.934 | 44.009 | 1.00 | 39.16 | C |
| ATOM | 1999 | CB | ARG | B | 5 | 32.877 | 40.538 | 45.072 | 1.00 | 42.51 | C |
| ATOM | 2000 | CG | ARG | B | 5 | 31.945 | 41.690 | 45.509 | 1.00 | 45.80 | C |
| ATOM | 2001 | CD | ARG | B | 5 | 30.809 | 41.289 | 46.440 | 1.00 | 37.82 | C |
| ATOM | 2002 | NE | ARG | B | 5 | 31.140 | 41.571 | 47.834 | 1.00 | 35.73 | N |
| ATOM | 2003 | CZ | ARG | B | 5 | 30.564 | 42.528 | 48.562 | 1.00 | 50.13 | C |
| ATOM | 2004 | NH1 | ARG | B | 5 | 29.600 | 43.293 | 48.029 | 1.00 | 17.38 | N |
| ATOM | 2005 | NH2 | ARG | B | 5 | 30.963 | 42.715 | 49.826 | 1.00 | 41.24 | N |
| ATOM | 2006 | C | ARG | B | 5 | 35.023 | 41.772 | 44.649 | 1.00 | 38.23 | C |
| ATOM | 2007 | O | ARG | B | 5 | 35.718 | 41.327 | 45.558 | 1.00 | 40.88 | O |
| ATOM | 2008 | N | LEU | B | 6 | 35.170 | 43.003 | 44.195 | 1.00 | 35.53 | N |
| ATOM | 2009 | CA | LEU | B | 6 | 36.238 | 43.829 | 44.711 | 1.00 | 37.55 | C |
| ATOM | 2010 | CB | LEU | B | 6 | 37.064 | 44.379 | 43.558 | 1.00 | 44.69 | C |
| ATOM | 2011 | CG | LEU | B | 6 | 38.340 | 45.103 | 43.962 | 1.00 | 44.94 | C |
| ATOM | 2012 | CD1 | LEU | B | 6 | 39.193 | 44.087 | 44.686 | 1.00 | 45.69 | C |
| ATOM | 2013 | CD2 | LEU | B | 6 | 39.008 | 45.592 | 42.694 | 1.00 | 57.99 | C |
| ATOM | 2014 | C | LEU | B | 6 | 35.646 | 44.967 | 45.506 | 1.00 | 33.43 | C |
| ATOM | 2015 | O | LEU | B | 6 | 34.918 | 45.792 | 44.962 | 1.00 | 29.84 | O |
| ATOM | 2016 | N | VAL | B | 7 | 35.978 | 45.010 | 46.788 | 1.00 | 29.77 | N |
| ATOM | 2017 | CA | VAL | B | 7 | 35.444 | 46.045 | 47.640 | 1.00 | 39.46 | C |
| ATOM | 2018 | CB | VAL | B | 7 | 35.680 | 45.734 | 49.117 | 1.00 | 45.72 | C |
| ATOM | 2019 | CG1 | VAL | B | 7 | 35.750 | 47.026 | 49.951 | 1.00 | 48.02 | C |
| ATOM | 2020 | CG2 | VAL | B | 7 | 34.552 | 44.862 | 49.610 | 1.00 | 27.64 | C |
| ATOM | 2021 | C | VAL | B | 7 | 35.991 | 47.420 | 47.291 | 1.00 | 43.72 | C |
| ATOM | 2022 | O | VAL | B | 7 | 35.222 | 48.376 | 47.148 | 1.00 | 45.73 | O |
| ATOM | 2023 | N | GLN | B | 8 | 37.317 | 47.529 | 47.199 | 1.00 | 49.20 | N |
| ATOM | 2024 | CA | GLN | B | 8 | 37.946 | 48.737 | 46.644 | 1.00 | 51.25 | C |
| ATOM | 2025 | CB | GLN | B | 8 | 39.437 | 48.802 | 47.005 | 1.00 | 39.27 | C |
| ATOM | 2026 | CG | GLN | B | 8 | 39.758 | 49.104 | 48.467 | 1.00 | 67.01 | C |
| ATOM | 2027 | CD | GLN | B | 8 | 41.258 | 49.352 | 48.719 | 1.00 | 84.83 | C |
| ATOM | 2028 | OE1 | GLN | B | 8 | 41.770 | 50.432 | 48.417 | 1.00 | 80.10 | O |
| ATOM | 2029 | NE2 | GLN | B | 8 | 41.944 | 48.370 | 49.312 | 1.00 | 76.98 | N |
| ATOM | 2030 | C | GLN | B | 8 | 37.799 | 48.760 | 45.114 | 1.00 | 53.17 | C |
| ATOM | 2031 | O | GLN | B | 8 | 38.615 | 48.176 | 44.392 | 1.00 | 62.83 | O |
| ATOM | 2032 | N | GLY | B | 9 | 36.738 | 49.376 | 44.604 | 1.00 | 43.64 | N |
| ATOM | 2033 | CA | GLY | B | 9 | 36.566 | 49.417 | 43.164 | 1.00 | 37.66 | C |
| ATOM | 2034 | C | GLY | B | 9 | 37.654 | 50.281 | 42.546 | 1.00 | 40.73 | C |
| ATOM | 2035 | O | GLY | B | 9 | 37.907 | 50.223 | 41.346 | 1.00 | 32.74 | O |
| ATOM | 2036 | N | SER | B | 10 | 38.203 | 51.189 | 43.354 | 1.00 | 39.45 | N |
| ATOM | 2037 | CA | SER | B | 10 | 39.025 | 52.242 | 42.809 | 1.00 | 30.93 | C |
| ATOM | 2038 | CB | SER | B | 10 | 39.489 | 53.165 | 43.939 | 1.00 | 42.43 | C |
| ATOM | 2039 | OG | SER | B | 10 | 39.922 | 52.393 | 45.049 | 1.00 | 58.97 | O |
| ATOM | 2040 | C | SER | B | 10 | 40.202 | 51.552 | 42.142 | 1.00 | 29.29 | C |
| ATOM | 2041 | O | SER | B | 10 | 40.861 | 52.113 | 41.263 | 1.00 | 40.95 | O |
| ATOM | 2042 | N | ILE | B | 11 | 40.541 | 50.373 | 42.637 | 1.00 | 27.61 | N |
| ATOM | 2043 | CA | ILE | B | 11 | 41.780 | 49.730 | 42.206 | 1.00 | 30.67 | C |
| ATOM | 2044 | CB | ILE | B | 11 | 42.112 | 48.500 | 43.129 | 1.00 | 34.34 | C |
| ATOM | 2045 | CG1 | ILE | B | 11 | 42.806 | 48.956 | 44.431 | 1.00 | 48.69 | C |
| ATOM | 2046 | CD1 | ILE | B | 11 | 42.920 | 47.858 | 45.496 | 1.00 | 45.19 | C |
| ATOM | 2047 | CG2 | ILE | B | 11 | 42.970 | 47.456 | 42.418 | 1.00 | 22.93 | C |
| ATOM | 2048 | C | ILE | B | 11 | 41.575 | 49.339 | 40.741 | 1.00 | 20.96 | C |
| ATOM | 2049 | O | ILE | B | 11 | 42.414 | 49.561 | 39.855 | 1.00 | 23.03 | O |
| ATOM | 2050 | N | LEU | B | 12 | 40.382 | 48.851 | 40.464 | 1.00 | 19.94 | N |
| ATOM | 2051 | CA | LEU | B | 12 | 40.128 | 48.352 | 39.132 | 1.00 | 28.47 | C |
| ATOM | 2052 | CB | LEU | B | 12 | 38.857 | 47.523 | 39.135 | 1.00 | 30.25 | C |
| ATOM | 2053 | CG | LEU | B | 12 | 38.659 | 46.616 | 37.916 | 1.00 | 34.67 | C |
| ATOM | 2054 | CD1 | LEU | B | 12 | 39.899 | 45.747 | 37.653 | 1.00 | 51.11 | C |
| ATOM | 2055 | CD2 | LEU | B | 12 | 37.420 | 45.738 | 38.108 | 1.00 | 31.87 | C |
| ATOM | 2056 | C | LEU | B | 12 | 40.018 | 49.479 | 38.107 | 1.00 | 30.05 | C |
| ATOM | 2057 | O | LEU | B | 12 | 40.306 | 49.316 | 36.933 | 1.00 | 36.51 | O |
| ATOM | 2058 | N | LYS | B | 13 | 39.602 | 50.647 | 38.553 | 1.00 | 31.99 | N |
| ATOM | 2059 | CA | LYS | B | 13 | 39.584 | 51.807 | 37.665 | 1.00 | 32.36 | C |
| ATOM | 2060 | CB | LYS | B | 13 | 38.717 | 52.927 | 38.268 | 1.00 | 33.99 | C |
| ATOM | 2061 | CG | LYS | B | 13 | 37.209 | 52.630 | 38.298 | 1.00 | 25.65 | C |
| ATOM | 2062 | CD | LYS | B | 13 | 36.507 | 53.328 | 39.466 | 1.00 | 39.64 | C |
| ATOM | 2063 | CE | LYS | B | 13 | 36.772 | 54.831 | 39.502 | 1.00 | 42.34 | C |
| ATOM | 2064 | NZ | LYS | B | 13 | 35.994 | 55.497 | 38.421 | 1.00 | 23.32 | N |
| ATOM | 2065 | C | LYS | B | 13 | 41.017 | 52.317 | 37.460 | 1.00 | 36.83 | C |
| ATOM | 2066 | O | LYS | B | 13 | 41.312 | 52.927 | 36.432 | 1.00 | 41.10 | O |
| ATOM | 2067 | N | LYS | B | 14 | 41.864 | 52.170 | 38.484 | 1.00 | 33.46 | N |
| ATOM | 2068 | CA | LYS | B | 14 | 43.206 | 52.735 | 38.443 | 1.00 | 31.02 | C |
| ATOM | 2069 | CB | LYS | B | 14 | 43.878 | 52.712 | 39.836 | 1.00 | 21.37 | C |
| ATOM | 2070 | CG | LYS | B | 14 | 43.533 | 53.921 | 40.668 | 1.00 | 13.48 | C |
| ATOM | 2071 | CD | LYS | B | 14 | 43.802 | 53.746 | 42.147 | 1.00 | 28.28 | C |
| ATOM | 2072 | CE | LYS | B | 14 | 43.290 | 54.936 | 42.989 | 1.00 | 42.47 | C |
| ATOM | 2073 | NZ | LYS | B | 14 | 44.337 | 55.996 | 43.318 | 1.00 | 48.32 | N |
| ATOM | 2074 | C | LYS | B | 14 | 43.915 | 51.846 | 37.454 | 1.00 | 32.28 | C |
| ATOM | 2075 | O | LYS | B | 14 | 44.785 | 52.310 | 36.708 | 1.00 | 34.88 | O |
| ATOM | 2076 | N | VAL | B | 15 | 43.516 | 50.575 | 37.459 | 1.00 | 23.57 | N |
| ATOM | 2077 | CA | VAL | B | 15 | 44.195 | 49.566 | 36.668 | 1.00 | 26.53 | C |
| ATOM | 2078 | CB | VAL | B | 15 | 43.690 | 48.168 | 36.977 | 1.00 | 27.23 | C |
| ATOM | 2079 | CG1 | VAL | B | 15 | 44.134 | 47.213 | 35.851 | 1.00 | 26.09 | C |
| ATOM | 2080 | CG2 | VAL | B | 15 | 44.214 | 47.723 | 38.339 | 1.00 | 42.73 | C |
| ATOM | 2081 | C | VAL | B | 15 | 43.995 | 49.804 | 35.185 | 1.00 | 21.24 | C |
| ATOM | 2082 | O | VAL | B | 15 | 44.941 | 49.762 | 34.387 | 1.00 | 17.32 | O |
| ATOM | 2083 | N | LEU | B | 16 | 42.754 | 50.123 | 34.850 | 1.00 | 23.19 | N |
| ATOM | 2084 | CA | LEU | B | 16 | 42.327 | 50.282 | 33.467 | 1.00 | 29.90 | C |
| ATOM | 2085 | CB | LEU | B | 16 | 40.810 | 50.121 | 33.349 | 1.00 | 36.32 | C |
| ATOM | 2086 | CG | LEU | B | 16 | 40.242 | 49.068 | 32.382 | 1.00 | 42.87 | C |
| ATOM | 2087 | CD1 | LEU | B | 16 | 40.668 | 47.670 | 32.802 | 1.00 | 41.52 | C |
| ATOM | 2088 | CD2 | LEU | B | 16 | 38.733 | 49.132 | 32.275 | 1.00 | 35.92 | C |
| ATOM | 2089 | C | LEU | B | 16 | 42.735 | 51.631 | 32.895 | 1.00 | 35.24 | C |
| ATOM | 2090 | O | LEU | B | 16 | 42.912 | 51.764 | 31.690 | 1.00 | 50.12 | O |
| ATOM | 2091 | N | GLU | B | 17 | 42.986 | 52.611 | 33.750 | 1.00 | 31.95 | N |
| ATOM | 2092 | CA | GLU | B | 17 | 43.490 | 53.891 | 33.261 | 1.00 | 32.04 | C |
| ATOM | 2093 | CB | GLU | B | 17 | 43.338 | 54.990 | 34.328 | 1.00 | 36.18 | C |
| ATOM | 2094 | CG | GLU | B | 17 | 41.943 | 55.595 | 34.406 | 1.00 | 51.43 | C |
| ATOM | 2095 | CD | GLU | B | 17 | 41.451 | 56.046 | 33.040 | 1.00 | 81.97 | C |
| ATOM | 2096 | OE1 | GLU | B | 17 | 41.887 | 57.134 | 32.595 | 1.00 | 83.39 | O |
| ATOM | 2097 | OE2 | GLU | B | 17 | 40.663 | 55.301 | 32.401 | 1.00 | 80.33 | O |
| ATOM | 2098 | C | GLU | B | 17 | 44.942 | 53.784 | 32.820 | 1.00 | 33.76 | C |
| ATOM | 2099 | O | GLU | B | 17 | 45.533 | 54.777 | 32.375 | 1.00 | 37.93 | O |
| ATOM | 2100 | N | ALA | B | 18 | 45.526 | 52.607 | 33.046 | 1.00 | 37.13 | N |
| ATOM | 2101 | CA | ALA | B | 18 | 46.972 | 52.416 | 32.992 | 1.00 | 38.80 | C |
| ATOM | 2102 | CB | ALA | B | 18 | 47.466 | 51.592 | 34.181 | 1.00 | 36.29 | C |
| ATOM | 2103 | C | ALA | B | 18 | 47.278 | 51.690 | 31.719 | 1.00 | 39.61 | C |
| ATOM | 2104 | O | ALA | B | 18 | 48.377 | 51.803 | 31.165 | 1.00 | 51.36 | O |
| ATOM | 2105 | N | LEU | B | 19 | 46.293 | 50.931 | 31.266 | 1.00 | 35.27 | N |
| ATOM | 2106 | CA | LEU | B | 19 | 46.524 | 50.034 | 30.154 | 1.00 | 33.66 | C |
| ATOM | 2107 | CB | LEU | B | 19 | 45.796 | 48.718 | 30.388 | 1.00 | 32.95 | C |
| ATOM | 2108 | CG | LEU | B | 19 | 45.994 | 48.156 | 31.793 | 1.00 | 35.48 | C |
| ATOM | 2109 | CD1 | LEU | B | 19 | 45.021 | 47.041 | 32.007 | 1.00 | 6.63 | C |
| ATOM | 2110 | CD2 | LEU | B | 19 | 47.432 | 47.671 | 31.989 | 1.00 | 40.73 | C |
| ATOM | 2111 | C | LEU | B | 19 | 46.033 | 50.716 | 28.894 | 1.00 | 35.31 | C |
| ATOM | 2112 | O | LEU | B | 19 | 46.593 | 50.547 | 27.810 | 1.00 | 39.07 | O |
| ATOM | 2113 | N | LYS | B | 20 | 45.000 | 51.528 | 29.048 | 1.00 | 36.70 | N |
| ATOM | 2114 | CA | LYS | B | 20 | 44.225 | 51.932 | 27.883 | 1.00 | 36.76 | C |
| ATOM | 2115 | CB | LYS | B | 20 | 42.912 | 52.629 | 28.274 | 1.00 | 25.54 | C |
| ATOM | 2116 | CG | LYS | B | 20 | 43.072 | 53.888 | 29.102 | 1.00 | 38.78 | C |
| ATOM | 2117 | CD | LYS | B | 20 | 42.167 | 55.004 | 28.603 | 1.00 | 33.36 | C |
| ATOM | 2118 | CE | LYS | B | 20 | 42.436 | 56.289 | 29.366 | 1.00 | 39.94 | C |
| ATOM | 2119 | NZ | LYS | B | 20 | 42.136 | 57.489 | 28.529 | 1.00 | 73.63 | N |
| ATOM | 2120 | C | LYS | B | 20 | 45.074 | 52.801 | 26.965 | 1.00 | 40.89 | C |
| ATOM | 2121 | O | LYS | B | 20 | 44.765 | 52.940 | 25.769 | 1.00 | 45.05 | O |
| ATOM | 2122 | N | ASP | B | 21 | 46.125 | 53.397 | 27.536 | 1.00 | 44.12 | N |
| ATOM | 2123 | CA | ASP | B | 21 | 46.957 | 54.356 | 26.805 | 1.00 | 46.45 | C |
| ATOM | 2124 | CB | ASP | B | 21 | 47.445 | 55.492 | 27.719 | 1.00 | 46.18 | C |
| ATOM | 2125 | CG | ASP | B | 21 | 46.352 | 56.517 | 28.016 | 1.00 | 62.51 | C |
| ATOM | 2126 | OD1 | ASP | B | 21 | 45.499 | 56.748 | 27.131 | 1.00 | 66.24 | O |
| ATOM | 2127 | OD2 | ASP | B | 21 | 46.244 | 57.116 | 29.111 | 1.00 | 69.18 | O |
| ATOM | 2128 | C | ASP | B | 21 | 48.132 | 53.620 | 26.164 | 1.00 | 43.38 | C |
| ATOM | 2129 | O | ASP | B | 21 | 48.713 | 54.083 | 25.184 | 1.00 | 45.39 | O |
| ATOM | 2130 | N | LEU | B | 22 | 48.385 | 52.412 | 26.655 | 1.00 | 43.62 | N |
| ATOM | 2131 | CA | LEU | B | 22 | 49.438 | 51.567 | 26.112 | 1.00 | 41.76 | C |
| ATOM | 2132 | CB | LEU | B | 22 | 50.104 | 50.747 | 27.213 | 1.00 | 37.49 | C |
| ATOM | 2133 | CG | LEU | B | 22 | 51.359 | 50.019 | 26.729 | 1.00 | 40.75 | C |
| ATOM | 2134 | CD1 | LEU | B | 22 | 52.353 | 50.955 | 26.048 | 1.00 | 41.88 | C |
| ATOM | 2135 | CD2 | LEU | B | 22 | 52.033 | 49.350 | 27.885 | 1.00 | 10.60 | C |
| ATOM | 2136 | C | LEU | B | 22 | 48.928 | 50.613 | 25.058 | 1.00 | 46.19 | C |
| ATOM | 2137 | O | LEU | B | 22 | 49.491 | 50.533 | 23.965 | 1.00 | 52.68 | O |
| ATOM | 2138 | N | ILE | B | 23 | 47.894 | 49.859 | 25.418 | 1.00 | 50.29 | N |
| ATOM | 2139 | CA | ILE | B | 23 | 47.260 | 48.902 | 24.517 | 1.00 | 48.77 | C |
| ATOM | 2140 | CB | ILE | B | 23 | 47.591 | 47.475 | 24.961 | 1.00 | 51.18 | C |
| ATOM | 2141 | CG1 | ILE | B | 23 | 47.207 | 47.259 | 26.424 | 1.00 | 46.32 | C |
| ATOM | 2142 | CD1 | ILE | B | 23 | 47.511 | 45.863 | 26.938 | 1.00 | 50.33 | C |
| ATOM | 2143 | CG2 | ILE | B | 23 | 49.085 | 47.244 | 24.837 | 1.00 | 63.93 | C |
| ATOM | 2144 | C | ILE | B | 23 | 45.755 | 49.110 | 24.494 | 1.00 | 50.04 | C |
| ATOM | 2145 | O | ILE | B | 23 | 45.233 | 49.861 | 25.319 | 1.00 | 55.43 | O |
| ATOM | 2146 | N | ASN | B | 24 | 45.072 | 48.471 | 23.539 | 1.00 | 51.80 | N |
| ATOM | 2147 | CA | ASN | B | 24 | 43.595 | 48.438 | 23.487 | 1.00 | 51.51 | C |
| ATOM | 2148 | CB | ASN | B | 24 | 43.065 | 49.448 | 22.481 | 1.00 | 41.61 | C |
| ATOM | 2149 | CG | ASN | B | 24 | 44.075 | 49.752 | 21.404 | 1.00 | 71.14 | C |
| ATOM | 2150 | OD1 | ASN | B | 24 | 45.154 | 50.283 | 21.697 | 1.00 | 84.83 | O |
| ATOM | 2151 | ND2 | ASN | B | 24 | 43.770 | 49.355 | 20.162 | 1.00 | 85.06 | N |
| ATOM | 2152 | C | ASN | B | 24 | 42.964 | 47.087 | 23.162 | 1.00 | 50.71 | C |
| ATOM | 2153 | O | ASN | B | 24 | 41.735 | 46.978 | 23.091 | 1.00 | 51.19 | O |
| ATOM | 2154 | N | GLU | B | 25 | 43.790 | 46.084 | 22.873 | 1.00 | 57.79 | N |
| ATOM | 2155 | CA | GLU | B | 25 | 43.274 | 44.756 | 22.550 | 1.00 | 58.92 | C |
| ATOM | 2156 | CB | GLU | B | 25 | 43.487 | 44.437 | 21.073 | 1.00 | 62.77 | C |
| ATOM | 2157 | CG | GLU | B | 25 | 42.804 | 45.409 | 20.129 | 1.00 | 71.89 | C |
| ATOM | 2158 | CD | GLU | B | 25 | 41.427 | 44.933 | 19.713 | 1.00 | 88.52 | C |
| ATOM | 2159 | OE1 | GLU | B | 25 | 41.346 | 44.068 | 18.812 | 1.00 | 101.56 | O |
| ATOM | 2160 | OE2 | GLU | B | 25 | 40.430 | 45.433 | 20.278 | 1.00 | 93.83 | O |
| ATOM | 2161 | C | GLU | B | 25 | 44.056 | 43.772 | 23.373 | 1.00 | 55.84 | C |
| ATOM | 2162 | O | GLU | B | 25 | 45.245 | 43.572 | 23.122 | 1.00 | 64.99 | O |
| ATOM | 2163 | N | ALA | B | 26 | 43.421 | 43.247 | 24.416 | 1.00 | 51.10 | N |
| ATOM | 2164 | CA | ALA | B | 26 | 44.106 | 42.364 | 25.348 | 1.00 | 47.49 | C |
| ATOM | 2165 | CB | ALA | B | 26 | 44.745 | 43.135 | 26.501 | 1.00 | 42.81 | C |
| ATOM | 2166 | C | ALA | B | 26 | 43.154 | 41.324 | 25.875 | 1.00 | 47.79 | C |
| ATOM | 2167 | O | ALA | B | 26 | 41.939 | 41.483 | 25.796 | 1.00 | 45.03 | O |
| ATOM | 2168 | N | CYS | B | 27 | 43.741 | 40.221 | 26.333 | 1.00 | 55.94 | N |
| ATOM | 2169 | CA | CYS | B | 27 | 43.005 | 39.043 | 26.771 | 1.00 | 57.72 | C |
| ATOM | 2170 | CB | CYS | B | 27 | 43.604 | 37.782 | 26.115 | 1.00 | 54.93 | C |
| ATOM | 2171 | SG | CYS | B | 27 | 42.742 | 36.226 | 26.462 | 1.00 | 87.85 | S |
| ATOM | 2172 | C | CYS | B | 27 | 43.142 | 38.976 | 28.286 | 1.00 | 46.55 | C |
| ATOM | 2173 | O | CYS | B | 27 | 44.243 | 39.094 | 28.816 | 1.00 | 45.39 | O |
| ATOM | 2174 | N | TRP | B | 28 | 42.013 | 38.787 | 28.960 | 1.00 | 46.05 | N |
| ATOM | 2175 | CA | TRP | B | 28 | 41.964 | 38.488 | 30.393 | 1.00 | 52.67 | C |
| ATOM | 2176 | CB | TRP | B | 28 | 40.729 | 39.159 | 30.988 | 1.00 | 57.39 | C |
| ATOM | 2177 | CG | TRP | B | 28 | 40.750 | 40.641 | 30.869 | 1.00 | 59.07 | C |
| ATOM | 2178 | CD1 | TRP | B | 28 | 40.742 | 41.392 | 29.716 | 1.00 | 56.71 | C |
| ATOM | 2179 | NE1 | TRP | B | 28 | 40.807 | 42.729 | 30.027 | 1.00 | 49.07 | N |
| ATOM | 2180 | CE2 | TRP | B | 28 | 40.861 | 42.862 | 31.395 | 1.00 | 51.08 | C |
| ATOM | 2181 | CD2 | TRP | B | 28 | 40.832 | 41.564 | 31.950 | 1.00 | 50.79 | C |
| ATOM | 2182 | CE3 | TRP | B | 28 | 40.885 | 41.426 | 33.339 | 1.00 | 48.98 | C |
| ATOM | 2183 | CZ3 | TRP | B | 28 | 40.926 | 42.563 | 34.119 | 1.00 | 59.03 | C |
| ATOM | 2184 | CH2 | TRP | B | 28 | 40.972 | 43.836 | 33.541 | 1.00 | 53.22 | C |
| ATOM | 2185 | CZ2 | TRP | B | 28 | 40.971 | 44.005 | 32.185 | 1.00 | 33.88 | C |
| ATOM | 2186 | C | TRP | B | 28 | 41.966 | 36.989 | 30.757 | 1.00 | 53.74 | C |
| ATOM | 2187 | O | TRP | B | 28 | 40.984 | 36.268 | 30.542 | 1.00 | 51.25 | O |
| ATOM | 2188 | N | ASP | B | 29 | 43.051 | 36.539 | 31.373 | 1.00 | 49.82 | N |
| ATOM | 2189 | CA | ASP | B | 29 | 43.157 | 35.153 | 31.756 | 1.00 | 52.44 | C |
| ATOM | 2190 | CB | ASP | B | 29 | 44.602 | 34.714 | 31.667 | 1.00 | 59.40 | C |
| ATOM | 2191 | CG | ASP | B | 29 | 45.162 | 34.949 | 30.301 | 1.00 | 70.22 | C |
| ATOM | 2192 | OD1 | ASP | B | 29 | 44.346 | 34.907 | 29.349 | 1.00 | 57.36 | O |
| ATOM | 2193 | OD2 | ASP | B | 29 | 46.361 | 35.247 | 30.090 | 1.00 | 87.62 | O |
| ATOM | 2194 | C | ASP | B | 29 | 42.648 | 34.988 | 33.162 | 1.00 | 57.24 | C |
| ATOM | 2195 | O | ASP | B | 29 | 43.228 | 35.488 | 34.118 | 1.00 | 59.22 | O |
| ATOM | 2196 | N | ILE | B | 30 | 41.547 | 34.265 | 33.280 | 1.00 | 62.56 | N |
| ATOM | 2197 | CA | ILE | B | 30 | 40.871 | 34.126 | 34.558 | 1.00 | 62.36 | C |
| ATOM | 2198 | CB | ILE | B | 30 | 39.416 | 34.557 | 34.399 | 1.00 | 60.50 | C |
| ATOM | 2199 | CG1 | ILE | B | 30 | 39.353 | 35.866 | 33.605 | 1.00 | 57.13 | C |
| ATOM | 2200 | CD1 | ILE | B | 30 | 38.533 | 36.960 | 34.264 | 1.00 | 72.51 | C |
| ATOM | 2201 | CG2 | ILE | B | 30 | 38.747 | 34.650 | 35.760 | 1.00 | 67.74 | C |
| ATOM | 2202 | C | ILE | B | 30 | 40.890 | 32.670 | 34.955 | 1.00 | 62.49 | C |
| ATOM | 2203 | O | ILE | B | 30 | 40.386 | 31.822 | 34.206 | 1.00 | 66.44 | O |
| ATOM | 2204 | N | SER | B | 31 | 41.435 | 32.387 | 36.136 | 1.00 | 52.84 | N |
| ATOM | 2205 | CA | SER | B | 31 | 41.407 | 31.025 | 36.649 | 1.00 | 59.30 | C |
| ATOM | 2206 | CB | SER | B | 31 | 42.373 | 30.139 | 35.851 | 1.00 | 56.63 | C |
| ATOM | 2207 | OG | SER | B | 31 | 43.725 | 30.374 | 36.232 | 1.00 | 56.69 | O |
| ATOM | 2208 | C | SER | B | 31 | 41.797 | 30.988 | 38.114 | 1.00 | 67.19 | C |
| ATOM | 2209 | O | SER | B | 31 | 42.921 | 31.357 | 38.465 | 1.00 | 73.85 | O |
| ATOM | 2210 | N | SER | B | 32 | 40.920 | 30.440 | 38.951 | 1.00 | 66.18 | N |
| ATOM | 2211 | CA | SER | B | 32 | 41.326 | 30.040 | 40.296 | 1.00 | 66.82 | C |
| ATOM | 2212 | CB | SER | B | 32 | 41.907 | 28.633 | 40.269 | 1.00 | 67.07 | C |
| ATOM | 2213 | OG | SER | B | 32 | 40.993 | 27.735 | 40.864 | 1.00 | 79.29 | O |
| ATOM | 2214 | C | SER | B | 32 | 42.314 | 30.991 | 40.992 | 1.00 | 69.87 | C |
| ATOM | 2215 | O | SER | B | 32 | 41.924 | 31.716 | 41.925 | 1.00 | 71.14 | O |
| ATOM | 2216 | N | SER | B | 33 | 43.591 | 30.958 | 40.586 | 1.00 | 70.03 | N |
| ATOM | 2217 | CA | SER | B | 33 | 44.629 | 31.793 | 41.217 | 1.00 | 70.78 | C |
| ATOM | 2218 | CB | SER | B | 33 | 45.965 | 31.642 | 40.467 | 1.00 | 75.26 | C |
| ATOM | 2219 | OG | SER | B | 33 | 46.274 | 32.809 | 39.691 | 1.00 | 81.47 | O |
| ATOM | 2220 | C | SER | B | 33 | 44.236 | 33.275 | 41.233 | 1.00 | 66.32 | C |
| ATOM | 2221 | O | SER | B | 33 | 44.467 | 34.009 | 42.205 | 1.00 | 52.37 | O |
| ATOM | 2222 | N | GLY | B | 34 | 43.732 | 33.728 | 40.090 | 1.00 | 64.47 | N |
| ATOM | 2223 | CA | GLY | B | 34 | 43.141 | 35.043 | 40.008 | 1.00 | 63.54 | C |
| ATOM | 2224 | C | GLY | B | 34 | 42.975 | 35.519 | 38.582 | 1.00 | 57.52 | C |
| ATOM | 2225 | O | GLY | B | 34 | 42.668 | 34.739 | 37.671 | 1.00 | 54.45 | O |
| ATOM | 2226 | N | VAL | B | 35 | 43.163 | 36.825 | 38.411 | 1.00 | 53.40 | N |
| ATOM | 2227 | CA | VAL | B | 35 | 43.173 | 37.437 | 37.093 | 1.00 | 57.39 | C |
| ATOM | 2228 | CB | VAL | B | 35 | 42.500 | 38.804 | 37.129 | 1.00 | 61.28 | C |
| ATOM | 2229 | CG1 | VAL | B | 35 | 41.731 | 39.043 | 35.821 | 1.00 | 54.10 | C |
| ATOM | 2230 | CG2 | VAL | B | 35 | 41.628 | 38.940 | 38.394 | 1.00 | 60.79 | C |
| ATOM | 2231 | C | VAL | B | 35 | 44.589 | 37.638 | 36.569 | 1.00 | 57.60 | C |
| ATOM | 2232 | O | VAL | B | 35 | 45.489 | 38.082 | 37.293 | 1.00 | 53.12 | O |
| ATOM | 2233 | N | ASN | B | 36 | 44.781 | 37.320 | 35.296 | 1.00 | 54.12 | N |
| ATOM | 2234 | CA | ASN | B | 36 | 46.046 | 37.641 | 34.634 | 1.00 | 60.84 | C |
| ATOM | 2235 | CB | ASN | B | 36 | 46.945 | 36.394 | 34.486 | 1.00 | 51.30 | C |
| ATOM | 2236 | CG | ASN | B | 36 | 47.185 | 35.712 | 35.816 | 1.00 | 53.71 | C |
| ATOM | 2237 | OD1 | ASN | B | 36 | 47.666 | 36.346 | 36.766 | 1.00 | 83.37 | O |
| ATOM | 2238 | ND2 | ASN | B | 36 | 46.733 | 34.462 | 35.941 | 1.00 | 61.84 | N |
| ATOM | 2239 | C | ASN | B | 36 | 45.830 | 38.356 | 33.291 | 1.00 | 63.87 | C |
| ATOM | 2240 | O | ASN | B | 36 | 44.809 | 38.149 | 32.601 | 1.00 | 58.00 | O |
| ATOM | 2241 | N | LEU | B | 37 | 46.773 | 39.236 | 32.957 | 1.00 | 56.04 | N |
| ATOM | 2242 | CA | LEU | B | 37 | 46.780 | 39.815 | 31.628 | 1.00 | 50.23 | C |
| ATOM | 2243 | CB | LEU | B | 37 | 46.077 | 41.159 | 31.651 | 1.00 | 48.09 | C |
| ATOM | 2244 | CG | LEU | B | 37 | 45.823 | 41.721 | 30.250 | 1.00 | 48.23 | C |
| ATOM | 2245 | CD1 | LEU | B | 37 | 44.333 | 41.755 | 29.983 | 1.00 | 59.37 | C |
| ATOM | 2246 | CD2 | LEU | B | 37 | 46.429 | 43.125 | 30.162 | 1.00 | 58.12 | C |
| ATOM | 2247 | C | LEU | B | 37 | 48.201 | 39.969 | 31.128 | 1.00 | 54.57 | C |
| ATOM | 2248 | O | LEU | B | 37 | 49.086 | 40.381 | 31.881 | 1.00 | 59.53 | O |
| ATOM | 2249 | N | GLN | B | 38 | 48.419 | 39.568 | 29.878 | 1.00 | 52.63 | N |
| ATOM | 2250 | CA | GLN | B | 38 | 49.715 | 39.707 | 29.219 | 1.00 | 48.07 | C |
| ATOM | 2251 | CB | GLN | B | 38 | 50.465 | 38.366 | 29.271 | 1.00 | 39.88 | C |
| ATOM | 2252 | CG | GLN | B | 38 | 51.275 | 38.150 | 30.559 | 1.00 | 58.58 | C |
| ATOM | 2253 | CD | GLN | B | 38 | 52.586 | 37.383 | 30.326 | 1.00 | 81.27 | C |
| ATOM | 2254 | OE1 | GLN | B | 38 | 52.914 | 37.036 | 29.181 | 1.00 | 76.64 | O |
| ATOM | 2255 | NE2 | GLN | B | 38 | 53.353 | 37.152 | 31.404 | 1.00 | 61.56 | N |
| ATOM | 2256 | C | GLN | B | 38 | 49.510 | 40.195 | 27.766 | 1.00 | 48.48 | C |
| ATOM | 2257 | O | GLN | B | 38 | 48.930 | 39.478 | 26.934 | 1.00 | 52.95 | O |
| ATOM | 2258 | N | SER | B | 39 | 49.919 | 41.433 | 27.472 | 1.00 | 38.67 | N |
| ATOM | 2259 | CA | SER | B | 39 | 49.875 | 41.914 | 26.091 | 1.00 | 38.78 | C |
| ATOM | 2260 | CB | SER | B | 39 | 48.520 | 42.552 | 25.783 | 1.00 | 37.43 | C |
| ATOM | 2261 | OG | SER | B | 39 | 48.004 | 42.102 | 24.531 | 1.00 | 36.53 | O |
| ATOM | 2262 | C | SER | B | 39 | 51.047 | 42.825 | 25.687 | 1.00 | 36.01 | C |
| ATOM | 2263 | O | SER | B | 39 | 51.771 | 43.326 | 26.534 | 1.00 | 44.79 | O |
| ATOM | 2264 | N | MET | B | 40 | 51.343 | 42.907 | 24.394 | 1.00 | 31.14 | N |
| ATOM | 2265 | CA | MET | B | 40 | 52.329 | 43.865 | 23.912 | 1.00 | 25.47 | C |
| ATOM | 2266 | CB | MET | B | 40 | 53.284 | 43.212 | 22.915 | 1.00 | 25.70 | C |
| ATOM | 2267 | CG | MET | B | 40 | 53.910 | 41.909 | 23.400 | 1.00 | 51.14 | C |
| ATOM | 2268 | SD | MET | B | 40 | 54.409 | 40.845 | 22.032 | 1.00 | 56.45 | S |
| ATOM | 2269 | CE | MET | B | 40 | 55.998 | 41.493 | 21.752 | 1.00 | 43.69 | C |
| ATOM | 2270 | C | MET | B | 40 | 51.628 | 45.006 | 23.203 | 1.00 | 23.26 | C |
| ATOM | 2271 | O | MET | B | 40 | 50.428 | 44.957 | 22.914 | 1.00 | 34.33 | O |
| ATOM | 2272 | N | ASP | B | 41 | 52.403 | 46.004 | 22.826 | 1.00 | 7.05 | N |
| ATOM | 2273 | CA | ASP | B | 41 | 51.840 | 47.115 | 22.099 | 1.00 | 20.66 | C |
| ATOM | 2274 | CB | ASP | B | 41 | 52.610 | 48.389 | 22.407 | 1.00 | 15.52 | C |
| ATOM | 2275 | CG | ASP | B | 41 | 54.034 | 48.342 | 21.882 | 1.00 | 33.70 | C |
| ATOM | 2276 | OD1 | ASP | B | 41 | 54.848 | 47.509 | 22.358 | 1.00 | 49.10 | O |
| ATOM | 2277 | OD2 | ASP | B | 41 | 54.439 | 49.121 | 21.000 | 1.00 | 23.93 | O |
| ATOM | 2278 | C | ASP | B | 41 | 51.846 | 46.815 | 20.588 | 1.00 | 29.82 | C |
| ATOM | 2279 | O | ASP | B | 41 | 52.142 | 45.683 | 20.170 | 1.00 | 30.84 | O |
| ATOM | 2280 | N | SER | B | 42 | 51.444 | 47.823 | 19.795 | 1.00 | 29.03 | N |
| ATOM | 2281 | CA | SER | B | 42 | 51.490 | 47.757 | 18.347 | 1.00 | 26.78 | C |
| ATOM | 2282 | CB | SER | B | 42 | 51.063 | 49.106 | 17.770 | 1.00 | 24.34 | C |
| ATOM | 2283 | OG | SER | B | 42 | 49.678 | 49.092 | 17.506 | 1.00 | 34.81 | O |
| ATOM | 2284 | C | SER | B | 42 | 52.893 | 47.379 | 17.837 | 1.00 | 34.67 | C |
| ATOM | 2285 | O | SER | B | 42 | 53.049 | 46.443 | 17.059 | 1.00 | 36.47 | O |
| ATOM | 2286 | N | SER | B | 43 | 53.907 | 48.164 | 18.205 | 1.00 | 42.26 | N |
| ATOM | 2287 | CA | SER | B | 43 | 55.249 | 47.987 | 17.656 | 1.00 | 38.04 | C |
| ATOM | 2288 | CB | SER | B | 43 | 56.036 | 49.296 | 17.674 | 1.00 | 41.38 | C |
| ATOM | 2289 | OG | SER | B | 43 | 56.294 | 49.709 | 19.020 | 1.00 | 43.61 | O |
| ATOM | 2290 | C | SER | B | 43 | 56.020 | 46.936 | 18.425 | 1.00 | 33.08 | C |
| ATOM | 2291 | O | SER | B | 43 | 57.190 | 46.734 | 18.165 | 1.00 | 26.01 | O |
| ATOM | 2292 | N | HIS | B | 44 | 55.338 | 46.228 | 19.320 | 1.00 | 42.99 | N |
| ATOM | 2293 | CA | HIS | B | 44 | 55.918 | 45.111 | 20.066 | 1.00 | 40.84 | C |
| ATOM | 2294 | CB | HIS | B | 44 | 56.201 | 43.915 | 19.160 | 1.00 | 42.68 | C |
| ATOM | 2295 | CG | HIS | B | 44 | 54.977 | 43.144 | 18.773 | 1.00 | 53.93 | C |
| ATOM | 2296 | ND1 | HIS | B | 44 | 55.021 | 41.815 | 18.411 | 1.00 | 24.39 | N |
| ATOM | 2297 | CE1 | HIS | B | 44 | 53.802 | 41.404 | 18.106 | 1.00 | 49.18 | C |
| ATOM | 2298 | NE2 | HIS | B | 44 | 52.978 | 42.435 | 18.189 | 1.00 | 57.65 | N |
| ATOM | 2299 | CD2 | HIS | B | 44 | 53.692 | 43.542 | 18.583 | 1.00 | 67.48 | C |
| ATOM | 2300 | C | HIS | B | 44 | 57.201 | 45.491 | 20.771 | 1.00 | 41.62 | C |
| ATOM | 2301 | O | HIS | B | 44 | 58.090 | 44.650 | 20.918 | 1.00 | 43.38 | O |
| ATOM | 2302 | N | VAL | B | 45 | 57.302 | 46.735 | 21.236 | 1.00 | 37.01 | N |
| ATOM | 2303 | CA | VAL | B | 45 | 58.445 | 47.092 | 22.065 | 1.00 | 35.09 | C |
| ATOM | 2304 | CB | VAL | B | 45 | 58.901 | 48.519 | 21.874 | 1.00 | 32.26 | C |
| ATOM | 2305 | CG1 | VAL | B | 45 | 60.350 | 48.596 | 22.259 | 1.00 | 14.70 | C |
| ATOM | 2306 | CG2 | VAL | B | 45 | 58.675 | 48.959 | 20.449 | 1.00 | 47.40 | C |
| ATOM | 2307 | C | VAL | B | 45 | 58.199 | 46.914 | 23.550 | 1.00 | 43.04 | C |
| ATOM | 2308 | O | VAL | B | 45 | 59.136 | 46.568 | 24.276 | 1.00 | 42.69 | O |
| ATOM | 2309 | N | SER | B | 46 | 56.983 | 47.225 | 24.009 | 1.00 | 40.95 | N |
| ATOM | 2310 | CA | SER | B | 46 | 56.663 | 47.112 | 25.429 | 1.00 | 38.77 | C |
| ATOM | 2311 | CB | SER | B | 46 | 56.049 | 48.397 | 25.965 | 1.00 | 39.96 | C |
| ATOM | 2312 | OG | SER | B | 46 | 54.724 | 48.529 | 25.505 | 1.00 | 41.86 | O |
| ATOM | 2313 | C | SER | B | 46 | 55.728 | 45.958 | 25.707 | 1.00 | 46.75 | C |
| ATOM | 2314 | O | SER | B | 46 | 55.118 | 45.405 | 24.798 | 1.00 | 50.55 | O |
| ATOM | 2315 | N | LEU | B | 47 | 55.621 | 45.599 | 26.980 | 1.00 | 55.68 | N |
| ATOM | 2316 | CA | LEU | B | 47 | 54.757 | 44.506 | 27.418 | 1.00 | 50.83 | C |
| ATOM | 2317 | CB | LEU | B | 47 | 55.576 | 43.226 | 27.549 | 1.00 | 42.79 | C |
| ATOM | 2318 | CG | LEU | B | 47 | 54.725 | 41.962 | 27.612 | 1.00 | 52.85 | C |
| ATOM | 2319 | CD1 | LEU | B | 47 | 55.296 | 40.873 | 26.717 | 1.00 | 54.91 | C |
| ATOM | 2320 | CD2 | LEU | B | 47 | 54.691 | 41.479 | 29.038 | 1.00 | 59.58 | C |
| ATOM | 2321 | C | LEU | B | 47 | 54.103 | 44.838 | 28.759 | 1.00 | 53.65 | C |
| ATOM | 2322 | O | LEU | B | 47 | 54.721 | 45.465 | 29.634 | 1.00 | 59.26 | O |
| ATOM | 2323 | N | VAL | B | 48 | 52.854 | 44.416 | 28.933 | 1.00 | 50.81 | N |
| ATOM | 2324 | CA | VAL | B | 48 | 52.205 | 44.540 | 30.238 | 1.00 | 41.25 | C |
| ATOM | 2325 | CB | VAL | B | 48 | 50.809 | 45.135 | 30.142 | 1.00 | 32.23 | C |
| ATOM | 2326 | CG1 | VAL | B | 48 | 50.554 | 45.996 | 31.364 | 1.00 | 47.43 | C |
| ATOM | 2327 | CG2 | VAL | B | 48 | 50.635 | 45.923 | 28.854 | 1.00 | 53.78 | C |
| ATOM | 2328 | C | VAL | B | 48 | 52.059 | 43.178 | 30.867 | 1.00 | 45.14 | C |
| ATOM | 2329 | O | VAL | B | 48 | 51.796 | 42.191 | 30.183 | 1.00 | 50.62 | O |
| ATOM | 2330 | N | GLN | B | 49 | 52.188 | 43.124 | 32.180 | 1.00 | 46.62 | N |
| ATOM | 2331 | CA | GLN | B | 49 | 51.939 | 41.868 | 32.857 | 1.00 | 52.81 | C |
| ATOM | 2332 | CB | GLN | B | 49 | 53.267 | 41.209 | 33.211 | 1.00 | 57.31 | C |
| ATOM | 2333 | CG | GLN | B | 49 | 53.178 | 39.709 | 33.500 | 1.00 | 54.93 | C |
| ATOM | 2334 | CD | GLN | B | 49 | 53.004 | 39.439 | 34.984 | 1.00 | 63.37 | C |
| ATOM | 2335 | OE1 | GLN | B | 49 | 53.970 | 39.441 | 35.752 | 1.00 | 63.69 | O |
| ATOM | 2336 | NE2 | GLN | B | 49 | 51.764 | 39.258 | 35.401 | 1.00 | 57.02 | N |
| ATOM | 2337 | C | GLN | B | 49 | 51.127 | 42.114 | 34.110 | 1.00 | 54.20 | C |
| ATOM | 2338 | O | GLN | B | 49 | 51.692 | 42.492 | 35.133 | 1.00 | 63.76 | O |
| ATOM | 2339 | N | LEU | B | 50 | 49.809 | 41.961 | 34.018 | 1.00 | 53.57 | N |
| ATOM | 2340 | CA | LEU | B | 50 | 48.937 | 42.229 | 35.164 | 1.00 | 54.29 | C |
| ATOM | 2341 | CB | LEU | B | 50 | 47.727 | 43.078 | 34.767 | 1.00 | 55.04 | C |
| ATOM | 2342 | CG | LEU | B | 50 | 46.649 | 43.145 | 35.855 | 1.00 | 36.80 | C |
| ATOM | 2343 | CD1 | LEU | B | 50 | 47.020 | 44.232 | 36.867 | 1.00 | 31.44 | C |
| ATOM | 2344 | CD2 | LEU | B | 50 | 45.270 | 43.392 | 35.226 | 1.00 | 29.39 | C |
| ATOM | 2345 | C | LEU | B | 50 | 48.462 | 40.968 | 35.884 | 1.00 | 52.19 | C |
| ATOM | 2346 | O | LEU | B | 50 | 47.993 | 40.008 | 35.246 | 1.00 | 38.43 | O |
| ATOM | 2347 | N | THR | B | 51 | 48.618 | 40.997 | 37.213 | 1.00 | 44.86 | N |
| ATOM | 2348 | CA | THR | B | 51 | 48.281 | 39.887 | 38.112 | 1.00 | 43.82 | C |
| ATOM | 2349 | CB | THR | B | 51 | 49.540 | 39.197 | 38.662 | 1.00 | 38.96 | C |
| ATOM | 2350 | OG1 | THR | B | 51 | 50.245 | 38.553 | 37.593 | 1.00 | 46.75 | O |
| ATOM | 2351 | CG2 | THR | B | 51 | 49.142 | 38.030 | 39.542 | 1.00 | 55.96 | C |
| ATOM | 2352 | C | THR | B | 51 | 47.451 | 40.356 | 39.299 | 1.00 | 43.36 | C |
| ATOM | 2353 | O | THR | B | 51 | 47.954 | 41.048 | 40.194 | 1.00 | 41.52 | O |
| ATOM | 2354 | N | LEU | B | 52 | 46.214 | 39.870 | 39.350 | 1.00 | 44.94 | N |
| ATOM | 2355 | CA | LEU | B | 52 | 45.329 | 40.106 | 40.478 | 1.00 | 44.71 | C |
| ATOM | 2356 | CB | LEU | B | 52 | 44.020 | 40.733 | 39.986 | 1.00 | 44.64 | C |
| ATOM | 2357 | CG | LEU | B | 52 | 44.202 | 42.194 | 39.554 | 1.00 | 47.53 | C |
| ATOM | 2358 | CD1 | LEU | B | 52 | 42.919 | 42.823 | 39.138 | 1.00 | 6.40 | C |
| ATOM | 2359 | CD2 | LEU | B | 52 | 44.878 | 43.017 | 40.659 | 1.00 | 51.56 | C |
| ATOM | 2360 | C | LEU | B | 52 | 45.039 | 38.770 | 41.137 | 1.00 | 49.25 | C |
| ATOM | 2361 | O | LEU | B | 52 | 44.366 | 37.920 | 40.549 | 1.00 | 50.21 | O |
| ATOM | 2362 | N | ARG | B | 53 | 45.553 | 38.582 | 42.348 | 1.00 | 47.29 | N |
| ATOM | 2363 | CA | ARG | B | 53 | 45.342 | 37.332 | 43.060 | 1.00 | 52.27 | C |
| ATOM | 2364 | CB | ARG | B | 53 | 46.304 | 37.246 | 44.245 | 1.00 | 52.57 | C |
| ATOM | 2365 | CG | ARG | B | 53 | 47.742 | 36.896 | 43.869 | 1.00 | 51.65 | C |
| ATOM | 2366 | CD | ARG | B | 53 | 48.661 | 36.747 | 45.079 | 1.00 | 46.01 | C |
| ATOM | 2367 | NE | ARG | B | 53 | 49.824 | 37.622 | 45.014 | 1.00 | 42.25 | N |
| ATOM | 2368 | CZ | ARG | B | 53 | 49.880 | 38.826 | 45.566 | 1.00 | 77.14 | C |
| ATOM | 2369 | NH1 | ARG | B | 53 | 48.826 | 39.316 | 46.211 | 1.00 | 70.32 | N |
| ATOM | 2370 | NH2 | ARG | B | 53 | 50.987 | 39.555 | 45.448 | 1.00 | 85.02 | N |
| ATOM | 2371 | C | ARG | B | 53 | 43.906 | 37.241 | 43.566 | 1.00 | 55.38 | C |
| ATOM | 2372 | O | ARG | B | 53 | 43.303 | 38.256 | 43.952 | 1.00 | 59.92 | O |
| ATOM | 2373 | N | SER | B | 54 | 43.398 | 36.012 | 43.657 | 1.00 | 54.38 | N |
| ATOM | 2374 | CA | SER | B | 54 | 42.116 | 35.750 | 44.328 | 1.00 | 49.63 | C |
| ATOM | 2375 | CB | SER | B | 54 | 41.639 | 34.329 | 44.010 | 1.00 | 44.81 | C |
| ATOM | 2376 | OG | SER | B | 54 | 42.527 | 33.346 | 44.533 | 1.00 | 43.77 | O |
| ATOM | 2377 | C | SER | B | 54 | 42.090 | 36.006 | 45.849 | 1.00 | 49.41 | C |
| ATOM | 2378 | O | SER | B | 54 | 41.030 | 36.239 | 46.423 | 1.00 | 50.98 | O |
| ATOM | 2379 | N | GLU | B | 55 | 43.246 | 35.926 | 46.503 | 1.00 | 45.93 | N |
| ATOM | 2380 | CA | GLU | B | 55 | 43.333 | 36.133 | 47.949 | 1.00 | 54.61 | C |
| ATOM | 2381 | CB | GLU | B | 55 | 44.779 | 35.864 | 48.446 | 1.00 | 55.01 | C |
| ATOM | 2382 | CG | GLU | B | 55 | 45.182 | 34.388 | 48.547 | 1.00 | 67.48 | C |
| ATOM | 2383 | CD | GLU | B | 55 | 45.476 | 33.741 | 47.187 | 1.00 | 85.08 | C |
| ATOM | 2384 | OE1 | GLU | B | 55 | 46.034 | 34.434 | 46.299 | 1.00 | 72.66 | O |
| ATOM | 2385 | OE2 | GLU | B | 55 | 45.186 | 32.526 | 47.007 | 1.00 | 74.58 | O |
| ATOM | 2386 | C | GLU | B | 55 | 42.911 | 37.577 | 48.276 | 1.00 | 53.60 | C |
| ATOM | 2387 | O | GLU | B | 55 | 42.877 | 37.996 | 49.451 | 1.00 | 52.89 | O |
| ATOM | 2388 | N | GLY | B | 56 | 42.699 | 38.363 | 47.219 | 1.00 | 49.62 | N |
| ATOM | 2389 | CA | GLY | B | 56 | 42.721 | 39.812 | 47.383 | 1.00 | 51.41 | C |
| ATOM | 2390 | C | GLY | B | 56 | 41.334 | 40.312 | 47.084 | 1.00 | 45.72 | C |
| ATOM | 2391 | O | GLY | B | 56 | 40.871 | 41.278 | 47.692 | 1.00 | 47.48 | O |
| ATOM | 2392 | N | PHE | B | 57 | 40.640 | 39.556 | 46.240 | 1.00 | 45.37 | N |
| ATOM | 2393 | CA | PHE | B | 57 | 39.179 | 39.668 | 46.083 | 1.00 | 52.55 | C |
| ATOM | 2394 | CB | PHE | B | 57 | 38.714 | 38.832 | 44.886 | 1.00 | 51.98 | C |
| ATOM | 2395 | CG | PHE | B | 57 | 38.955 | 39.497 | 43.557 | 1.00 | 60.50 | C |
| ATOM | 2396 | CD1 | PHE | B | 57 | 40.061 | 39.160 | 42.786 | 1.00 | 49.04 | C |
| ATOM | 2397 | CE1 | PHE | B | 57 | 40.327 | 39.811 | 41.601 | 1.00 | 41.48 | C |
| ATOM | 2398 | CZ | PHE | B | 57 | 39.503 | 40.837 | 41.190 | 1.00 | 62.57 | C |
| ATOM | 2399 | CE2 | PHE | B | 57 | 38.407 | 41.206 | 41.962 | 1.00 | 64.76 | C |
| ATOM | 2400 | CD2 | PHE | B | 57 | 38.132 | 40.527 | 43.127 | 1.00 | 70.26 | C |
| ATOM | 2401 | C | PHE | B | 57 | 38.283 | 39.350 | 47.298 | 1.00 | 54.29 | C |
| ATOM | 2402 | O | PHE | B | 57 | 38.418 | 38.307 | 47.947 | 1.00 | 61.82 | O |
| ATOM | 2403 | N | ASP | B | 58 | 37.312 | 40.214 | 47.567 | 1.00 | 50.97 | N |
| ATOM | 2404 | CA | ASP | B | 58 | 36.396 | 39.965 | 48.671 | 1.00 | 53.32 | C |
| ATOM | 2405 | CB | ASP | B | 58 | 35.428 | 41.146 | 48.832 | 1.00 | 57.78 | C |
| ATOM | 2406 | CG | ASP | B | 58 | 34.860 | 41.265 | 50.243 | 1.00 | 72.98 | C |
| ATOM | 2407 | OD1 | ASP | B | 58 | 35.619 | 41.633 | 51.168 | 1.00 | 75.02 | O |
| ATOM | 2408 | OD2 | ASP | B | 58 | 33.645 | 41.080 | 50.514 | 1.00 | 84.91 | O |
| ATOM | 2409 | C | ASP | B | 58 | 35.652 | 38.635 | 48.488 | 1.00 | 46.82 | C |
| ATOM | 2410 | O | ASP | B | 58 | 35.429 | 37.898 | 49.444 | 1.00 | 54.94 | O |
| ATOM | 2411 | N | THR | B | 59 | 35.317 | 38.296 | 47.256 | 1.00 | 34.54 | N |
| ATOM | 2412 | CA | THR | B | 59 | 34.640 | 37.041 | 47.003 | 1.00 | 36.39 | C |
| ATOM | 2413 | CB | THR | B | 59 | 33.132 | 37.232 | 47.056 | 1.00 | 38.58 | C |
| ATOM | 2414 | OG1 | THR | B | 59 | 32.782 | 37.787 | 48.326 | 1.00 | 46.59 | O |
| ATOM | 2415 | CG2 | THR | B | 59 | 32.422 | 35.871 | 47.016 | 1.00 | 42.36 | C |
| ATOM | 2416 | C | THR | B | 59 | 35.015 | 36.504 | 45.638 | 1.00 | 38.68 | C |
| ATOM | 2417 | O | THR | B | 59 | 34.400 | 36.842 | 44.629 | 1.00 | 53.30 | O |
| ATOM | 2418 | N | TYR | B | 60 | 35.949 | 35.577 | 45.606 | 1.00 | 34.77 | N |
| ATOM | 2419 | CA | TYR | B | 60 | 36.324 | 34.981 | 44.339 | 1.00 | 39.28 | C |
| ATOM | 2420 | CB | TYR | B | 60 | 37.849 | 34.873 | 44.283 | 1.00 | 41.30 | C |
| ATOM | 2421 | CG | TYR | B | 60 | 38.391 | 34.730 | 42.885 | 1.00 | 54.11 | C |
| ATOM | 2422 | CD1 | TYR | B | 60 | 38.783 | 35.840 | 42.161 | 1.00 | 68.06 | C |
| ATOM | 2423 | CE1 | TYR | B | 60 | 39.245 | 35.713 | 40.866 | 1.00 | 72.82 | C |
| ATOM | 2424 | CZ | TYR | B | 60 | 39.312 | 34.460 | 40.274 | 1.00 | 67.63 | C |
| ATOM | 2425 | OH | TYR | B | 60 | 39.788 | 34.333 | 38.988 | 1.00 | 45.12 | O |
| ATOM | 2426 | CE2 | TYR | B | 60 | 38.907 | 33.340 | 40.968 | 1.00 | 66.04 | C |
| ATOM | 2427 | CD2 | TYR | B | 60 | 38.430 | 33.485 | 42.257 | 1.00 | 71.52 | C |
| ATOM | 2428 | C | TYR | B | 60 | 35.638 | 33.627 | 44.063 | 1.00 | 40.41 | C |
| ATOM | 2429 | O | TYR | B | 60 | 35.375 | 32.840 | 44.973 | 1.00 | 46.67 | O |
| ATOM | 2430 | N | ARG | B | 61 | 35.342 | 33.353 | 42.801 | 1.00 | 37.62 | N |
| ATOM | 2431 | CA | ARG | B | 61 | 34.704 | 32.106 | 42.445 | 1.00 | 47.11 | C |
| ATOM | 2432 | CB | ARG | B | 61 | 33.198 | 32.147 | 42.744 | 1.00 | 52.92 | C |
| ATOM | 2433 | CG | ARG | B | 61 | 32.456 | 30.848 | 42.416 | 1.00 | 59.80 | C |
| ATOM | 2434 | CD | ARG | B | 61 | 30.985 | 30.854 | 42.771 | 1.00 | 59.61 | C |
| ATOM | 2435 | NE | ARG | B | 61 | 30.217 | 30.192 | 41.718 | 1.00 | 78.13 | N |
| ATOM | 2436 | CZ | ARG | B | 61 | 28.899 | 30.027 | 41.745 | 1.00 | 86.08 | C |
| ATOM | 2437 | NH1 | ARG | B | 61 | 28.184 | 30.473 | 42.780 | 1.00 | 95.72 | N |
| ATOM | 2438 | NH2 | ARG | B | 61 | 28.290 | 29.427 | 40.727 | 1.00 | 86.37 | N |
| ATOM | 2439 | C | ARG | B | 61 | 34.907 | 31.868 | 40.967 | 1.00 | 57.56 | C |
| ATOM | 2440 | O | ARG | B | 61 | 34.330 | 32.561 | 40.126 | 1.00 | 59.82 | O |
| ATOM | 2441 | N | CYS | B | 62 | 35.674 | 30.833 | 40.659 | 1.00 | 68.12 | N |
| ATOM | 2442 | CA | CYS | B | 62 | 35.885 | 30.416 | 39.279 | 1.00 | 71.50 | C |
| ATOM | 2443 | CB | CYS | B | 62 | 37.255 | 30.865 | 38.805 | 1.00 | 77.74 | C |
| ATOM | 2444 | SG | CYS | B | 62 | 37.350 | 30.770 | 37.000 | 1.00 | 88.93 | S |
| ATOM | 2445 | C | CYS | B | 62 | 35.782 | 28.917 | 39.072 | 1.00 | 71.42 | C |
| ATOM | 2446 | O | CYS | B | 62 | 36.562 | 28.126 | 39.614 | 1.00 | 69.28 | O |
| ATOM | 2447 | N | ASP | B | 63 | 34.840 | 28.525 | 38.233 | 1.00 | 78.13 | N |
| ATOM | 2448 | CA | ASP | B | 63 | 34.561 | 27.111 | 38.075 | 1.00 | 77.33 | C |
| ATOM | 2449 | CB | ASP | B | 63 | 33.142 | 26.907 | 37.518 | 1.00 | 77.09 | C |
| ATOM | 2450 | CG | ASP | B | 63 | 32.048 | 27.149 | 38.583 | 1.00 | 76.98 | C |
| ATOM | 2451 | OD1 | ASP | B | 63 | 32.267 | 27.914 | 39.552 | 1.00 | 71.49 | O |
| ATOM | 2452 | OD2 | ASP | B | 63 | 30.924 | 26.612 | 38.545 | 1.00 | 64.28 | O |
| ATOM | 2453 | C | ASP | B | 63 | 35.644 | 26.527 | 37.176 | 1.00 | 73.84 | C |
| ATOM | 2454 | O | ASP | B | 63 | 36.588 | 25.858 | 37.651 | 1.00 | 72.81 | O |
| ATOM | 2455 | N | ARG | B | 64 | 35.568 | 26.882 | 35.897 | 1.00 | 70.10 | N |
| ATOM | 2456 | CA | ARG | B | 64 | 36.594 | 26.471 | 34.948 | 1.00 | 72.51 | C |
| ATOM | 2457 | CB | ARG | B | 64 | 35.955 | 25.900 | 33.670 | 1.00 | 72.51 | C |
| ATOM | 2458 | CG | ARG | B | 64 | 34.501 | 26.348 | 33.460 | 1.00 | 81.63 | C |
| ATOM | 2459 | CD | ARG | B | 64 | 33.682 | 25.395 | 32.583 | 1.00 | 100.98 | C |
| ATOM | 2460 | NE | ARG | B | 64 | 32.807 | 24.512 | 33.365 | 1.00 | 96.58 | N |
| ATOM | 2461 | CZ | ARG | B | 64 | 32.920 | 23.182 | 33.408 | 1.00 | 108.13 | C |
| ATOM | 2462 | NH1 | ARG | B | 64 | 33.882 | 22.567 | 32.712 | 1.00 | 107.55 | N |
| ATOM | 2463 | NH2 | ARG | B | 64 | 32.089 | 22.466 | 34.169 | 1.00 | 87.67 | N |
| ATOM | 2464 | C | ARG | B | 64 | 37.572 | 27.607 | 34.618 | 1.00 | 72.42 | C |
| ATOM | 2465 | O | ARG | B | 64 | 37.471 | 28.724 | 35.156 | 1.00 | 59.60 | O |
| ATOM | 2466 | N | ASN | B | 65 | 38.528 | 27.294 | 33.740 | 1.00 | 73.87 | N |
| ATOM | 2467 | CA | ASN | B | 65 | 39.440 | 28.289 | 33.181 | 1.00 | 68.68 | C |
| ATOM | 2468 | CB | ASN | B | 65 | 40.658 | 27.625 | 32.523 | 1.00 | 63.37 | C |
| ATOM | 2469 | CG | ASN | B | 65 | 41.680 | 27.111 | 33.535 | 1.00 | 68.64 | C |
| ATOM | 2470 | OD1 | ASN | B | 65 | 42.725 | 26.579 | 33.152 | 1.00 | 69.90 | O |
| ATOM | 2471 | ND2 | ASN | B | 65 | 41.358 | 27.213 | 34.822 | 1.00 | 50.32 | N |
| ATOM | 2472 | C | ASN | B | 65 | 38.724 | 29.175 | 32.166 | 1.00 | 69.20 | C |
| ATOM | 2473 | O | ASN | B | 65 | 37.824 | 28.720 | 31.446 | 1.00 | 58.47 | O |
| ATOM | 2474 | N | LEU | B | 66 | 39.145 | 30.438 | 32.117 | 1.00 | 70.87 | N |
| ATOM | 2475 | CA | LEU | B | 66 | 38.580 | 31.418 | 31.196 | 1.00 | 67.80 | C |
| ATOM | 2476 | CB | LEU | B | 66 | 37.588 | 32.321 | 31.944 | 1.00 | 68.68 | C |
| ATOM | 2477 | CG | LEU | B | 66 | 36.116 | 32.288 | 31.513 | 1.00 | 71.82 | C |
| ATOM | 2478 | CD1 | LEU | B | 66 | 35.270 | 33.085 | 32.508 | 1.00 | 59.84 | C |
| ATOM | 2479 | CD2 | LEU | B | 66 | 35.910 | 32.791 | 30.069 | 1.00 | 84.53 | C |
| ATOM | 2480 | C | LEU | B | 66 | 39.675 | 32.252 | 30.505 | 1.00 | 58.60 | C |
| ATOM | 2481 | O | LEU | B | 66 | 40.726 | 32.531 | 31.091 | 1.00 | 53.08 | O |
| ATOM | 2482 | N | ALA | B | 67 | 39.408 | 32.652 | 29.263 | 1.00 | 48.99 | N |
| ATOM | 2483 | CA | ALA | B | 67 | 40.254 | 33.595 | 28.527 | 1.00 | 44.72 | C |
| ATOM | 2484 | CB | ALA | B | 67 | 41.131 | 32.832 | 27.521 | 1.00 | 31.81 | C |
| ATOM | 2485 | C | ALA | B | 67 | 39.393 | 34.640 | 27.802 | 1.00 | 46.92 | C |
| ATOM | 2486 | O | ALA | B | 67 | 38.760 | 34.336 | 26.783 | 1.00 | 48.29 | O |
| ATOM | 2487 | N | MET | B | 68 | 39.341 | 35.860 | 28.335 | 1.00 | 47.89 | N |
| ATOM | 2488 | CA | MET | B | 68 | 38.397 | 36.859 | 27.834 | 1.00 | 49.28 | C |
| ATOM | 2489 | CB | MET | B | 68 | 37.558 | 37.448 | 28.972 | 1.00 | 49.11 | C |
| ATOM | 2490 | CG | MET | B | 68 | 36.139 | 36.863 | 29.037 | 1.00 | 46.82 | C |
| ATOM | 2491 | SD | MET | B | 68 | 35.161 | 37.841 | 30.161 | 1.00 | 75.21 | S |
| ATOM | 2492 | CE | MET | B | 68 | 36.262 | 37.939 | 31.659 | 1.00 | 40.59 | C |
| ATOM | 2493 | C | MET | B | 68 | 39.013 | 37.966 | 26.957 | 1.00 | 50.26 | C |
| ATOM | 2494 | O | MET | B | 68 | 39.775 | 38.817 | 27.424 | 1.00 | 44.80 | O |
| ATOM | 2495 | N | GLY | B | 69 | 38.671 | 37.950 | 25.672 | 1.00 | 51.34 | N |
| ATOM | 2496 | CA | GLY | B | 69 | 39.116 | 38.990 | 24.770 | 1.00 | 49.91 | C |
| ATOM | 2497 | C | GLY | B | 69 | 38.329 | 40.263 | 24.985 | 1.00 | 50.59 | C |
| ATOM | 2498 | O | GLY | B | 69 | 37.190 | 40.406 | 24.518 | 1.00 | 44.96 | O |
| ATOM | 2499 | N | VAL | B | 70 | 38.970 | 41.215 | 25.651 | 1.00 | 51.26 | N |
| ATOM | 2500 | CA | VAL | B | 70 | 38.296 | 42.444 | 26.032 | 1.00 | 49.48 | C |
| ATOM | 2501 | CB | VAL | B | 70 | 38.333 | 42.626 | 27.543 | 1.00 | 51.07 | C |
| ATOM | 2502 | CG1 | VAL | B | 70 | 37.795 | 43.983 | 27.928 | 1.00 | 53.92 | C |
| ATOM | 2503 | CG2 | VAL | B | 70 | 37.553 | 41.516 | 28.217 | 1.00 | 49.93 | C |
| ATOM | 2504 | C | VAL | B | 70 | 38.955 | 43.639 | 25.369 | 1.00 | 45.92 | C |
| ATOM | 2505 | O | VAL | B | 70 | 40.170 | 43.790 | 25.440 | 1.00 | 46.61 | O |
| ATOM | 2506 | N | ASN | B | 71 | 38.150 | 44.452 | 24.687 | 1.00 | 45.25 | N |
| ATOM | 2507 | CA | ASN | B | 71 | 38.557 | 45.794 | 24.279 | 1.00 | 39.08 | C |
| ATOM | 2508 | CB | ASN | B | 71 | 37.580 | 46.356 | 23.263 | 1.00 | 33.36 | C |
| ATOM | 2509 | CG | ASN | B | 71 | 37.962 | 47.755 | 22.829 | 1.00 | 47.74 | C |
| ATOM | 2510 | OD1 | ASN | B | 71 | 39.131 | 48.145 | 22.899 | 1.00 | 58.14 | O |
| ATOM | 2511 | ND2 | ASN | B | 71 | 36.997 | 48.484 | 22.288 | 1.00 | 53.48 | N |
| ATOM | 2512 | C | ASN | B | 71 | 38.690 | 46.789 | 25.428 | 1.00 | 36.42 | C |
| ATOM | 2513 | O | ASN | B | 71 | 37.706 | 47.137 | 26.088 | 1.00 | 40.74 | O |
| ATOM | 2514 | N | LEU | B | 72 | 39.911 | 47.256 | 25.661 | 1.00 | 34.47 | N |
| ATOM | 2515 | CA | LEU | B | 72 | 40.228 | 47.982 | 26.881 | 1.00 | 37.62 | C |
| ATOM | 2516 | CB | LEU | B | 72 | 41.728 | 48.042 | 27.111 | 1.00 | 28.50 | C |
| ATOM | 2517 | CG | LEU | B | 72 | 42.318 | 46.694 | 27.516 | 1.00 | 35.19 | C |
| ATOM | 2518 | CD1 | LEU | B | 72 | 43.592 | 46.482 | 26.727 | 1.00 | 30.78 | C |
| ATOM | 2519 | CD2 | LEU | B | 72 | 42.573 | 46.670 | 29.012 | 1.00 | 20.88 | C |
| ATOM | 2520 | C | LEU | B | 72 | 39.633 | 49.377 | 26.848 | 1.00 | 41.40 | C |
| ATOM | 2521 | O | LEU | B | 72 | 39.212 | 49.912 | 27.875 | 1.00 | 55.17 | O |
| ATOM | 2522 | N | THR | B | 73 | 39.504 | 49.932 | 25.653 | 1.00 | 37.16 | N |
| ATOM | 2523 | CA | THR | B | 73 | 38.993 | 51.285 | 25.535 | 1.00 | 37.02 | C |
| ATOM | 2524 | CB | THR | B | 73 | 39.278 | 51.807 | 24.130 | 1.00 | 41.73 | C |
| ATOM | 2525 | OG1 | THR | B | 73 | 40.670 | 51.591 | 23.838 | 1.00 | 58.09 | O |
| ATOM | 2526 | CG2 | THR | B | 73 | 39.090 | 53.320 | 24.077 | 1.00 | 46.25 | C |
| ATOM | 2527 | C | THR | B | 73 | 37.505 | 51.240 | 25.812 | 1.00 | 34.84 | C |
| ATOM | 2528 | O | THR | B | 73 | 36.941 | 52.111 | 26.476 | 1.00 | 42.90 | O |
| ATOM | 2529 | N | SER | B | 74 | 36.861 | 50.196 | 25.325 | 1.00 | 28.44 | N |
| ATOM | 2530 | CA | SER | B | 74 | 35.467 | 50.036 | 25.665 | 1.00 | 33.72 | C |
| ATOM | 2531 | CB | SER | B | 74 | 34.896 | 48.803 | 24.953 | 1.00 | 38.05 | C |
| ATOM | 2532 | OG | SER | B | 74 | 34.755 | 49.072 | 23.569 | 1.00 | 51.33 | O |
| ATOM | 2533 | C | SER | B | 74 | 35.352 | 49.916 | 27.188 | 1.00 | 32.88 | C |
| ATOM | 2534 | O | SER | B | 74 | 34.468 | 50.486 | 27.844 | 1.00 | 27.81 | O |
| ATOM | 2535 | N | MET | B | 75 | 36.216 | 49.080 | 27.743 | 1.00 | 35.18 | N |
| ATOM | 2536 | CA | MET | B | 75 | 36.086 | 48.724 | 29.143 | 1.00 | 31.80 | C |
| ATOM | 2537 | CB | MET | B | 75 | 37.105 | 47.656 | 29.549 | 1.00 | 23.92 | C |
| ATOM | 2538 | CG | MET | B | 75 | 36.508 | 46.686 | 30.565 | 1.00 | 36.47 | C |
| ATOM | 2539 | SD | MET | B | 75 | 37.655 | 45.469 | 31.155 | 1.00 | 34.31 | S |
| ATOM | 2540 | CE | MET | B | 75 | 36.782 | 44.758 | 32.586 | 1.00 | 42.13 | C |
| ATOM | 2541 | C | MET | B | 75 | 36.266 | 49.951 | 29.997 | 1.00 | 28.54 | C |
| ATOM | 2542 | O | MET | B | 75 | 35.631 | 50.089 | 31.038 | 1.00 | 31.72 | O |
| ATOM | 2543 | N | SER | B | 76 | 37.156 | 50.828 | 29.549 | 1.00 | 26.69 | N |
| ATOM | 2544 | CA | SER | B | 76 | 37.573 | 51.967 | 30.354 | 1.00 | 31.79 | C |
| ATOM | 2545 | CB | SER | B | 76 | 38.852 | 52.590 | 29.793 | 1.00 | 31.36 | C |
| ATOM | 2546 | OG | SER | B | 76 | 38.624 | 53.162 | 28.514 | 1.00 | 54.99 | O |
| ATOM | 2547 | C | SER | B | 76 | 36.448 | 52.985 | 30.358 | 1.00 | 34.03 | C |
| ATOM | 2548 | O | SER | B | 76 | 36.235 | 53.696 | 31.342 | 1.00 | 31.61 | O |
| ATOM | 2549 | N | LYS | B | 77 | 35.705 | 53.024 | 29.258 | 1.00 | 35.05 | N |
| ATOM | 2550 | CA | LYS | B | 77 | 34.549 | 53.897 | 29.202 | 1.00 | 35.05 | C |
| ATOM | 2551 | CB | LYS | B | 77 | 33.981 | 53.919 | 27.776 | 1.00 | 32.43 | C |
| ATOM | 2552 | CG | LYS | B | 77 | 34.753 | 54.854 | 26.865 | 1.00 | 43.72 | C |
| ATOM | 2553 | CD | LYS | B | 77 | 34.225 | 54.862 | 25.442 | 1.00 | 59.30 | C |
| ATOM | 2554 | CE | LYS | B | 77 | 35.297 | 55.388 | 24.479 | 1.00 | 66.79 | C |
| ATOM | 2555 | NZ | LYS | B | 77 | 35.299 | 54.673 | 23.162 | 1.00 | 54.22 | N |
| ATOM | 2556 | C | LYS | B | 77 | 33.516 | 53.462 | 30.256 | 1.00 | 33.52 | C |
| ATOM | 2557 | O | LYS | B | 77 | 32.912 | 54.291 | 30.962 | 1.00 | 38.58 | O |
| ATOM | 2558 | N | ILE | B | 78 | 33.332 | 52.149 | 30.360 | 1.00 | 26.01 | N |
| ATOM | 2559 | CA | ILE | B | 78 | 32.360 | 51.610 | 31.269 | 1.00 | 29.13 | C |
| ATOM | 2560 | CB | ILE | B | 78 | 32.178 | 50.120 | 30.964 | 1.00 | 32.56 | C |
| ATOM | 2561 | CG1 | ILE | B | 78 | 31.603 | 49.946 | 29.562 | 1.00 | 35.08 | C |
| ATOM | 2562 | CD1 | ILE | B | 78 | 31.249 | 48.499 | 29.284 | 1.00 | 31.46 | C |
| ATOM | 2563 | CG2 | ILE | B | 78 | 31.203 | 49.485 | 31.964 | 1.00 | 50.02 | C |
| ATOM | 2564 | C | ILE | B | 78 | 32.838 | 51.845 | 32.698 | 1.00 | 25.69 | C |
| ATOM | 2565 | O | ILE | B | 78 | 32.074 | 82.264 | 33.583 | 1.00 | 22.92 | O |
| ATOM | 2566 | N | LEU | B | 79 | 34.103 | 51.540 | 32.931 | 1.00 | 23.13 | N |
| ATOM | 2567 | CA | LEU | B | 79 | 34.628 | 51.597 | 34.276 | 1.00 | 26.23 | C |
| ATOM | 2568 | CB | LEU | B | 79 | 35.970 | 50.886 | 34.348 | 1.00 | 16.09 | C |
| ATOM | 2569 | CG | LEU | B | 79 | 35.875 | 49.652 | 35.231 | 1.00 | 34.08 | C |
| ATOM | 2570 | CD1 | LEU | B | 79 | 35.908 | 48.406 | 34.383 | 1.00 | 54.70 | C |
| ATOM | 2571 | CD2 | LEU | B | 79 | 36.988 | 49.660 | 36.271 | 1.00 | 57.44 | C |
| ATOM | 2572 | C | LEU | B | 79 | 34.754 | 53.047 | 34.687 | 1.00 | 30.90 | C |
| ATOM | 2573 | O | LEU | B | 79 | 35.101 | 53.365 | 35.820 | 1.00 | 36.03 | O |
| ATOM | 2574 | N | LYS | B | 80 | 34.416 | 53.936 | 33.765 | 1.00 | 40.33 | N |
| ATOM | 2575 | CA | LYS | B | 80 | 34.492 | 55.352 | 34.081 | 1.00 | 41.16 | C |
| ATOM | 2576 | CB | LYS | B | 80 | 34.714 | 56.206 | 32.819 | 1.00 | 40.37 | C |
| ATOM | 2577 | CG | LYS | B | 80 | 36.166 | 56.706 | 32.636 | 1.00 | 35.36 | C |
| ATOM | 2578 | CD | LYS | B | 80 | 36.293 | 58.227 | 32.777 | 1.00 | 63.32 | C |
| ATOM | 2579 | CE | LYS | B | 80 | 36.394 | 58.934 | 31.412 | 1.00 | 71.45 | C |
| ATOM | 2580 | NZ | LYS | B | 80 | 35.284 | 59.922 | 31.159 | 1.00 | 31.08 | N |
| ATOM | 2581 | C | LYS | B | 80 | 33.220 | 55.721 | 34.819 | 1.00 | 36.12 | C |
| ATOM | 2582 | O | LYS | B | 80 | 33.135 | 56.774 | 35.456 | 1.00 | 36.76 | O |
| ATOM | 2583 | N | CYS | B | 81 | 32.273 | 54.788 | 34.801 | 1.00 | 32.51 | N |
| ATOM | 2584 | CA | CYS | B | 81 | 30.950 | 55.040 | 35.345 | 1.00 | 37.12 | C |
| ATOM | 2585 | CB | CYS | B | 81 | 29.892 | 54.285 | 34.555 | 1.00 | 36.87 | C |
| ATOM | 2586 | SG | CYS | B | 81 | 29.901 | 54.655 | 32.792 | 1.00 | 42.76 | S |
| ATOM | 2587 | C | CYS | B | 81 | 30.858 | 54.622 | 36.793 | 1.00 | 37.62 | C |
| ATOM | 2588 | O | CYS | B | 81 | 29.957 | 55.029 | 37.502 | 1.00 | 50.00 | O |
| ATOM | 2589 | N | ALA | B | 82 | 31.780 | 53.784 | 37.231 | 1.00 | 37.05 | N |
| ATOM | 2590 | CA | ALA | B | 82 | 31.811 | 53.363 | 38.626 | 1.00 | 31.80 | C |
| ATOM | 2591 | CB | ALA | B | 82 | 32.646 | 52.085 | 38.763 | 1.00 | 31.37 | C |
| ATOM | 2592 | C | ALA | B | 82 | 32.386 | 54.493 | 39.481 | 1.00 | 27.83 | C |
| ATOM | 2593 | O | ALA | B | 82 | 33.211 | 55.262 | 38.991 | 1.00 | 28.49 | O |
| ATOM | 2594 | N | GLY | B | 83 | 31.882 | 54.660 | 40.709 | 1.00 | 22.73 | N |
| ATOM | 2595 | CA | GLY | B | 83 | 32.389 | 55.684 | 41.624 | 1.00 | 19.66 | C |
| ATOM | 2596 | C | GLY | B | 83 | 33.578 | 55.166 | 42.425 | 1.00 | 33.84 | C |
| ATOM | 2597 | O | GLY | B | 83 | 34.006 | 54.010 | 42.271 | 1.00 | 47.28 | O |
| ATOM | 2598 | N | ASN | B | 84 | 34.177 | 56.033 | 43.231 | 1.00 | 40.44 | N |
| ATOM | 2599 | CA | ASN | B | 84 | 35.440 | 55.688 | 43.882 | 1.00 | 49.06 | C |
| ATOM | 2600 | CB | ASN | B | 84 | 36.039 | 56.919 | 44.551 | 1.00 | 58.63 | C |
| ATOM | 2601 | CG | ASN | B | 84 | 37.243 | 57.441 | 43.806 | 1.00 | 75.77 | C |
| ATOM | 2602 | OD1 | ASN | B | 84 | 38.387 | 57.137 | 44.166 | 1.00 | 93.56 | O |
| ATOM | 2603 | ND2 | ASN | B | 84 | 36.996 | 58.211 | 42.740 | 1.00 | 59.94 | N |
| ATOM | 2604 | C | ASN | B | 84 | 35.317 | 54.575 | 44.913 | 1.00 | 51.77 | C |
| ATOM | 2605 | O | ASN | B | 84 | 36.192 | 53.710 | 45.028 | 1.00 | 58.92 | O |
| ATOM | 2606 | N | GLU | B | 85 | 34.262 | 54.638 | 45.715 | 1.00 | 53.79 | N |
| ATOM | 2607 | CA | GLU | B | 85 | 34.056 | 53.631 | 46.744 | 1.00 | 57.87 | C |
| ATOM | 2608 | CB | GLU | B | 85 | 33.313 | 54.256 | 47.920 | 1.00 | 57.60 | C |
| ATOM | 2609 | CG | GLU | B | 85 | 33.009 | 55.726 | 47.682 | 1.00 | 62.68 | C |
| ATOM | 2610 | CD | GLU | B | 85 | 34.019 | 56.629 | 48.376 | 1.00 | 76.83 | C |
| ATOM | 2611 | OE1 | GLU | B | 85 | 33.629 | 57.354 | 49.326 | 1.00 | 46.85 | O |
| ATOM | 2612 | OE2 | GLU | B | 85 | 35.212 | 56.602 | 47.978 | 1.00 | 92.54 | O |
| ATOM | 2613 | C | GLU | B | 85 | 33.280 | 52.438 | 46.179 | 1.00 | 54.03 | C |
| ATOM | 2614 | O | GLU | B | 85 | 33.147 | 51.392 | 46.820 | 1.00 | 55.80 | O |
| ATOM | 2615 | N | ASP | B | 86 | 32.728 | 52.618 | 44.985 | 1.00 | 49.07 | N |
| ATOM | 2616 | CA | ASP | B | 86 | 31.749 | 51.665 | 44.479 | 1.00 | 46.20 | C |
| ATOM | 2617 | CB | ASP | B | 86 | 31.301 | 52.025 | 43.053 | 1.00 | 41.56 | C |
| ATOM | 2618 | CG | ASP | B | 86 | 30.195 | 53.075 | 43.030 | 1.00 | 59.22 | C |
| ATOM | 2619 | OD1 | ASP | B | 86 | 30.304 | 54.090 | 43.768 | 1.00 | 71.68 | O |
| ATOM | 2620 | OD2 | ASP | B | 86 | 29.161 | 52.947 | 42.327 | 1.00 | 73.17 | O |
| ATOM | 2621 | C | ASP | B | 86 | 32.366 | 50.280 | 44.520 | 1.00 | 38.44 | C |
| ATOM | 2622 | O | ASP | B | 86 | 33.483 | 50.061 | 44.003 | 1.00 | 39.12 | O |
| ATOM | 2623 | N | ILE | B | 87 | 31.661 | 49.345 | 45.137 | 1.00 | 33.66 | N |
| ATOM | 2624 | CA | ILE | B | 87 | 32.080 | 47.946 | 45.061 | 1.00 | 36.87 | C |
| ATOM | 2625 | CB | ILE | B | 87 | 31.362 | 47.144 | 46.182 | 1.00 | 37.69 | C |
| ATOM | 2626 | CG1 | ILE | B | 87 | 31.581 | 47.850 | 47.521 | 1.00 | 33.60 | C |
| ATOM | 2627 | CD1 | ILE | B | 87 | 30.325 | 47.976 | 48.390 | 1.00 | 44.49 | C |
| ATOM | 2628 | CG2 | ILE | B | 87 | 31.922 | 45.719 | 46.244 | 1.00 | 34.55 | C |
| ATOM | 2629 | C | ILE | B | 87 | 31.832 | 47.293 | 43.687 | 1.00 | 32.95 | C |
| ATOM | 2630 | O | ILE | B | 87 | 30.706 | 47.003 | 43.278 | 1.00 | 32.54 | O |
| ATOM | 2631 | N | ILE | B | 88 | 32.920 | 47.012 | 42.988 | 1.00 | 19.93 | N |
| ATOM | 2632 | CA | ILE | B | 88 | 32.766 | 46.566 | 41.624 | 1.00 | 32.40 | C |
| ATOM | 2633 | CB | ILE | B | 88 | 33.933 | 47.085 | 40.775 | 1.00 | 34.61 | C |
| ATOM | 2634 | CG1 | ILE | B | 88 | 33.799 | 48.609 | 40.586 | 1.00 | 45.43 | C |
| ATOM | 2635 | CD1 | ILE | B | 88 | 34.904 | 49.258 | 39.771 | 1.00 | 52.09 | C |
| ATOM | 2636 | CG2 | ILE | B | 88 | 34.049 | 46.304 | 39.469 | 1.00 | 37.20 | C |
| ATOM | 2637 | C | ILE | B | 88 | 32.740 | 45.060 | 41.657 | 1.00 | 38.41 | C |
| ATOM | 2638 | O | ILE | B | 88 | 33.518 | 44.425 | 42.371 | 1.00 | 54.68 | O |
| ATOM | 2639 | N | THR | B | 89 | 31.847 | 44.467 | 40.885 | 1.00 | 39.15 | N |
| ATOM | 2640 | CA | THR | B | 89 | 31.835 | 43.020 | 40.812 | 1.00 | 34.84 | C |
| ATOM | 2641 | CB | THR | B | 89 | 30.579 | 42.521 | 41.518 | 1.00 | 39.16 | C |
| ATOM | 2642 | OG1 | THR | B | 89 | 30.410 | 43.264 | 42.742 | 1.00 | 37.16 | O |
| ATOM | 2643 | CG2 | THR | B | 89 | 30.730 | 41.055 | 41.918 | 1.00 | 21.10 | C |
| ATOM | 2644 | C | THR | B | 89 | 31.817 | 42.600 | 39.353 | 1.00 | 35.21 | C |
| ATOM | 2645 | O | THR | B | 89 | 30.898 | 42.974 | 38.615 | 1.00 | 40.62 | O |
| ATOM | 2646 | N | LEU | B | 90 | 32.831 | 41.840 | 38.942 | 1.00 | 23.77 | N |
| ATOM | 2647 | CA | LEU | B | 90 | 32.868 | 41.234 | 37.603 | 1.00 | 24.28 | C |
| ATOM | 2648 | CB | LEU | B | 90 | 34.292 | 41.165 | 37.027 | 1.00 | 27.14 | C |
| ATOM | 2649 | CG | LEU | B | 90 | 35.223 | 42.317 | 37.469 | 1.00 | 49.12 | C |
| ATOM | 2650 | CD1 | LEU | B | 90 | 36.650 | 41.858 | 37.624 | 1.00 | 48.59 | C |
| ATOM | 2651 | CD2 | LEU | B | 90 | 35.167 | 43.585 | 36.593 | 1.00 | 25.48 | C |
| ATOM | 2652 | C | LEU | B | 90 | 32.278 | 39.842 | 37.630 | 1.00 | 29.91 | C |
| ATOM | 2653 | O | LEU | B | 90 | 32.461 | 39.092 | 38.591 | 1.00 | 41.72 | O |
| ATOM | 2654 | N | ARG | B | 91 | 31.547 | 39.498 | 36.578 | 1.00 | 32.80 | N |
| ATOM | 2655 | CA | ARG | B | 91 | 30.806 | 38.241 | 36.540 | 1.00 | 35.98 | C |
| ATOM | 2656 | CB | ARG | B | 91 | 29.415 | 38.423 | 37.183 | 1.00 | 41.51 | C |
| ATOM | 2657 | CG | ARG | B | 91 | 28.786 | 37.170 | 37.777 | 1.00 | 52.69 | C |
| ATOM | 2658 | CD | ARG | B | 91 | 27.475 | 37.431 | 38.513 | 1.00 | 70.75 | C |
| ATOM | 2659 | NE | ARG | B | 91 | 26.421 | 37.909 | 37.620 | 1.00 | 76.03 | N |
| ATOM | 2660 | CZ | ARG | B | 91 | 25.554 | 38.865 | 37.940 | 1.00 | 80.08 | C |
| ATOM | 2661 | NH1 | ARG | B | 91 | 25.625 | 39.458 | 39.132 | 1.00 | 61.14 | N |
| ATOM | 2662 | NH2 | ARG | B | 91 | 24.643 | 39.256 | 37.052 | 1.00 | 77.65 | N |
| ATOM | 2663 | C | ARG | B | 91 | 30.685 | 37.784 | 35.076 | 1.00 | 36.94 | C |
| ATOM | 2664 | O | ARG | B | 91 | 30.160 | 38.496 | 34.208 | 1.00 | 41.94 | O |
| ATOM | 2665 | N | ALA | B | 92 | 31.178 | 36.589 | 34.788 | 1.00 | 40.30 | N |
| ATOM | 2666 | CA | ALA | B | 92 | 30.949 | 35.994 | 33.476 | 1.00 | 42.74 | C |
| ATOM | 2667 | CB | ALA | B | 92 | 32.206 | 36.034 | 32.648 | 1.00 | 29.51 | C |
| ATOM | 2668 | C | ALA | B | 92 | 30.477 | 34.556 | 33.621 | 1.00 | 55.17 | C |
| ATOM | 2669 | O | ALA | B | 92 | 30.709 | 33.919 | 34.671 | 1.00 | 57.21 | O |
| ATOM | 2670 | N | GLU | B | 93 | 29.871 | 34.042 | 32.543 | 1.00 | 62.67 | N |
| ATOM | 2671 | CA | GLU | B | 93 | 29.454 | 32.641 | 32.478 | 1.00 | 70.74 | C |
| ATOM | 2672 | CB | GLU | B | 93 | 28.154 | 32.496 | 31.681 | 1.00 | 74.88 | C |
| ATOM | 2673 | CG | GLU | B | 93 | 26.909 | 32.977 | 32.427 | 1.00 | 80.84 | C |
| ATOM | 2674 | CD | GLU | B | 93 | 26.718 | 32.298 | 33.784 | 1.00 | 93.11 | C |
| ATOM | 2675 | OE1 | GLU | B | 93 | 26.566 | 31.052 | 33.792 | 1.00 | 105.85 | O |
| ATOM | 2676 | OE2 | GLU | B | 93 | 26.695 | 32.998 | 34.839 | 1.00 | 48.91 | O |
| ATOM | 2677 | C | GLU | B | 93 | 30.554 | 31.806 | 31.841 | 1.00 | 76.47 | C |
| ATOM | 2678 | O | GLU | B | 93 | 31.447 | 32.362 | 31.191 | 1.00 | 82.00 | O |
| ATOM | 2679 | N | ASP | B | 94 | 30.533 | 30.489 | 32.072 | 1.00 | 82.25 | N |
| ATOM | 2680 | CA | ASP | B | 94 | 31.605 | 29.618 | 31.579 | 1.00 | 83.03 | C |
| ATOM | 2681 | CB | ASP | B | 94 | 31.550 | 28.231 | 32.233 | 1.00 | 83.04 | C |
| ATOM | 2682 | CG | ASP | B | 94 | 31.704 | 28.304 | 33.746 | 1.00 | 93.18 | C |
| ATOM | 2683 | OD1 | ASP | B | 94 | 32.559 | 29.088 | 34.230 | 1.00 | 66.41 | O |
| ATOM | 2684 | OD2 | ASP | B | 94 | 30.981 | 27.644 | 34.528 | 1.00 | 102.18 | O |
| ATOM | 2685 | C | ASP | B | 94 | 31.583 | 29.498 | 30.056 | 1.00 | 80.28 | C |
| ATOM | 2686 | O | ASP | B | 94 | 32.607 | 29.242 | 29.424 | 1.00 | 74.89 | O |
| ATOM | 2687 | N | ASN | B | 95 | 30.432 | 29.784 | 29.462 | 1.00 | 79.41 | N |
| ATOM | 2688 | CA | ASN | B | 95 | 30.388 | 29.983 | 28.023 | 1.00 | 87.64 | C |
| ATOM | 2689 | CB | ASN | B | 95 | 29.268 | 29.136 | 27.392 | 1.00 | 92.69 | C |
| ATOM | 2690 | CG | ASN | B | 95 | 28.884 | 27.914 | 28.236 | 1.00 | 99.97 | C |
| ATOM | 2691 | OD1 | ASN | B | 95 | 29.663 | 26.967 | 28.366 | 1.00 | 106.49 | O |
| ATOM | 2692 | ND2 | ASN | B | 95 | 27.646 | 27.895 | 28.734 | 1.00 | 84.47 | N |
| ATOM | 2693 | C | ASN | B | 95 | 30.199 | 31.457 | 27.660 | 1.00 | 92.26 | C |
| ATOM | 2694 | O | ASN | B | 95 | 29.257 | 31.794 | 26.941 | 1.00 | 100.82 | O |
| ATOM | 2695 | N | ALA | B | 96 | 31.064 | 32.340 | 28.164 | 1.00 | 92.30 | N |
| ATOM | 2696 | CA | ALA | B | 96 | 30.787 | 33.787 | 28.147 | 1.00 | 84.54 | C |
| ATOM | 2697 | CB | ALA | B | 96 | 31.653 | 34.522 | 29.167 | 1.00 | 84.87 | C |
| ATOM | 2698 | C | ALA | B | 96 | 30.881 | 34.461 | 26.765 | 1.00 | 79.99 | C |
| ATOM | 2699 | O | ALA | B | 96 | 31.945 | 34.504 | 26.142 | 1.00 | 78.07 | O |
| ATOM | 2700 | N | ASP | B | 97 | 29.741 | 34.947 | 26.277 | 1.00 | 76.61 | N |
| ATOM | 2701 | CA | ASP | B | 97 | 29.683 | 35.775 | 25.075 | 1.00 | 69.20 | C |
| ATOM | 2702 | CB | ASP | B | 97 | 28.312 | 35.605 | 24.390 | 1.00 | 75.33 | C |
| ATOM | 2703 | CG | ASP | B | 97 | 28.182 | 36.396 | 23.085 | 1.00 | 86.50 | C |
| ATOM | 2704 | OD1 | ASP | B | 97 | 28.807 | 35.973 | 22.081 | 1.00 | 93.67 | O |
| ATOM | 2705 | OD2 | ASP | B | 97 | 27.397 | 37.375 | 22.943 | 1.00 | 90.23 | O |
| ATOM | 2706 | C | ASP | B | 97 | 29.901 | 37.228 | 25.493 | 1.00 | 59.03 | C |
| ATOM | 2707 | O | ASP | B | 97 | 30.478 | 38.006 | 24.752 | 1.00 | 58.07 | O |
| ATOM | 2708 | N | THR | B | 98 | 29.413 | 37.595 | 26.672 | 1.00 | 47.32 | N |
| ATOM | 2709 | CA | THR | B | 98 | 29.569 | 38.950 | 27.171 | 1.00 | 38.69 | C |
| ATOM | 2710 | CB | THR | B | 98 | 28.190 | 39.718 | 27.245 | 1.00 | 41.95 | C |
| ATOM | 2711 | OG1 | THR | B | 98 | 27.194 | 38.905 | 27.860 | 1.00 | 33.76 | O |
| ATOM | 2712 | CG2 | THR | B | 98 | 27.585 | 39.947 | 25.864 | 1.00 | 40.74 | C |
| ATOM | 2713 | C | THR | B | 98 | 30.259 | 38.924 | 28.533 | 1.00 | 37.17 | C |
| ATOM | 2714 | O | THR | B | 98 | 30.544 | 37.857 | 29.088 | 1.00 | 35.06 | O |
| ATOM | 2715 | N | LEU | B | 99 | 30.481 | 40.107 | 29.100 | 1.00 | 34.88 | N |
| ATOM | 2716 | CA | LEU | B | 99 | 31.010 | 40.213 | 30.453 | 1.00 | 39.86 | C |
| ATOM | 2717 | CB | LEU | B | 99 | 32.502 | 40.530 | 30.429 | 1.00 | 42.46 | C |
| ATOM | 2718 | CG | LEU | B | 99 | 33.071 | 40.942 | 31.783 | 1.00 | 48.98 | C |
| ATOM | 2719 | CD1 | LEU | B | 99 | 33.115 | 39.741 | 32.703 | 1.00 | 52.29 | C |
| ATOM | 2720 | CD2 | LEU | B | 99 | 34.452 | 41.561 | 31.635 | 1.00 | 44.95 | C |
| ATOM | 2721 | C | LEU | B | 99 | 30.272 | 41.281 | 31.240 | 1.00 | 41.36 | C |
| ATOM | 2722 | O | LEU | B | 99 | 30.082 | 42.407 | 30.753 | 1.00 | 44.28 | O |
| ATOM | 2723 | N | ALA | B | 100 | 29.788 | 40.898 | 32.419 | 1.00 | 31.42 | N |
| ATOM | 2724 | CA | ALA | B | 100 | 28.894 | 41.771 | 33.157 | 1.00 | 30.83 | C |
| ATOM | 2725 | CB | ALA | B | 100 | 27.785 | 40.973 | 33.843 | 1.00 | 33.84 | C |
| ATOM | 2726 | C | ALA | B | 100 | 29.735 | 42.548 | 34.158 | 1.00 | 36.59 | C |
| ATOM | 2727 | O | ALA | B | 100 | 30.836 | 42.127 | 34.509 | 1.00 | 42.48 | O |
| ATOM | 2728 | N | LEU | B | 101 | 29.265 | 43.731 | 34.537 | 1.00 | 34.53 | N |
| ATOM | 2729 | CA | LEU | B | 101 | 30.037 | 44.592 | 35.417 | 1.00 | 19.41 | C |
| ATOM | 2730 | CB | LEU | B | 101 | 30.712 | 45.654 | 34.575 | 1.00 | 21.32 | C |
| ATOM | 2731 | CG | LEU | B | 101 | 31.980 | 45.230 | 33.851 | 1.00 | 23.48 | C |
| ATOM | 2732 | CD1 | LEU | B | 101 | 31.774 | 45.561 | 32.391 | 1.00 | 9.89 | C |
| ATOM | 2733 | CD2 | LEU | B | 101 | 33.203 | 45.944 | 34.438 | 1.00 | 23.11 | C |
| ATOM | 2734 | C | LEU | B | 101 | 29.140 | 45.276 | 36.428 | 1.00 | 23.31 | C |
| ATOM | 2735 | O | LEU | B | 101 | 28.280 | 46.089 | 36.062 | 1.00 | 29.97 | O |
| ATOM | 2736 | N | VAL | B | 102 | 29.264 | 44.878 | 37.686 | 1.00 | 22.77 | N |
| ATOM | 2737 | CA | VAL | B | 102 | 28.317 | 45.352 | 38.686 | 1.00 | 29.20 | C |
| ATOM | 2738 | CB | VAL | B | 102 | 27.684 | 44.182 | 39.489 | 1.00 | 30.94 | C |
| ATOM | 2739 | CG1 | VAL | B | 102 | 26.523 | 44.685 | 40.396 | 1.00 | 38.64 | C |
| ATOM | 2740 | CG2 | VAL | B | 102 | 27.179 | 43.088 | 38.541 | 1.00 | 24.11 | C |
| ATOM | 2741 | C | VAL | B | 102 | 28.919 | 46.426 | 39.600 | 1.00 | 29.64 | C |
| ATOM | 2742 | O | VAL | B | 102 | 29.848 | 46.161 | 40.360 | 1.00 | 34.73 | O |
| ATOM | 2743 | N | PHE | B | 103 | 28.418 | 47.652 | 39.489 | 1.00 | 25.14 | N |
| ATOM | 2744 | CA | PHE | B | 103 | 28.913 | 48.760 | 40.295 | 1.00 | 23.37 | C |
| ATOM | 2745 | CB | PHE | B | 103 | 29.126 | 50.020 | 39.442 | 1.00 | 13.97 | C |
| ATOM | 2746 | CG | PHE | B | 103 | 30.021 | 49.805 | 38.272 | 1.00 | 27.78 | C |
| ATOM | 2747 | CD1 | PHE | B | 103 | 31.014 | 48.845 | 38.316 | 1.00 | 50.56 | C |
| ATOM | 2748 | CE1 | PHE | B | 103 | 31.814 | 48.598 | 37.218 | 1.00 | 35.73 | C |
| ATOM | 2749 | CZ | PHE | B | 103 | 31.633 | 49.311 | 36.069 | 1.00 | 44.33 | C |
| ATOM | 2750 | CE2 | PHE | B | 103 | 30.646 | 50.274 | 36.009 | 1.00 | 55.98 | C |
| ATOM | 2751 | CD2 | PHE | B | 103 | 29.845 | 50.521 | 37.108 | 1.00 | 44.57 | C |
| ATOM | 2752 | C | PHE | B | 103 | 27.902 | 49.043 | 41.404 | 1.00 | 28.34 | C |
| ATOM | 2753 | O | PHE | B | 103 | 26.874 | 49.716 | 41.213 | 1.00 | 23.18 | O |
| ATOM | 2754 | N | GLU | B | 104 | 28.232 | 48.591 | 42.603 | 1.00 | 35.30 | N |
| ATOM | 2755 | CA | GLU | B | 104 | 27.308 | 48.766 | 43.718 | 1.00 | 39.28 | C |
| ATOM | 2756 | CB | GLU | B | 104 | 27.009 | 47.413 | 44.337 | 1.00 | 44.42 | C |
| ATOM | 2757 | CG | GLU | B | 104 | 26.255 | 47.445 | 45.649 | 1.00 | 28.85 | C |
| ATOM | 2758 | CD | GLU | B | 104 | 26.478 | 46.154 | 46.419 | 1.00 | 48.41 | C |
| ATOM | 2759 | OE1 | GLU | B | 104 | 26.917 | 46.237 | 47.588 | 1.00 | 63.11 | O |
| ATOM | 2760 | OE2 | GLU | B | 104 | 26.289 | 45.057 | 45.836 | 1.00 | 35.71 | O |
| ATOM | 2761 | C | GLU | B | 104 | 27.881 | 49.695 | 44.768 | 1.00 | 41.78 | C |
| ATOM | 2762 | O | GLU | B | 104 | 29.048 | 49.553 | 45.151 | 1.00 | 48.20 | O |
| ATOM | 2763 | N | ALA | B | 105 | 27.070 | 50.671 | 45.176 | 1.00 | 41.47 | N |
| ATOM | 2764 | CA | ALA | B | 105 | 27.474 | 51.702 | 46.127 | 1.00 | 43.88 | C |
| ATOM | 2765 | CB | ALA | B | 105 | 26.561 | 52.887 | 46.051 | 1.00 | 43.73 | C |
| ATOM | 2766 | C | ALA | B | 105 | 27.422 | 51.130 | 47.522 | 1.00 | 53.32 | C |
| ATOM | 2767 | O | ALA | B | 105 | 26.676 | 50.182 | 47.777 | 1.00 | 48.98 | O |
| ATOM | 2768 | N | PRO | B | 106 | 28.311 | 51.619 | 48.385 | 1.00 | 62.19 | N |
| ATOM | 2769 | CA | PRO | B | 106 | 28.545 | 50.955 | 49.672 | 1.00 | 65.15 | C |
| ATOM | 2770 | CB | PRO | B | 106 | 29.291 | 52.025 | 50.484 | 1.00 | 63.91 | C |
| ATOM | 2771 | CG | PRO | B | 106 | 30.158 | 52.688 | 49.440 | 1.00 | 57.48 | C |
| ATOM | 2772 | CD | PRO | B | 106 | 29.235 | 52.750 | 48.177 | 1.00 | 63.03 | C |
| ATOM | 2773 | C | PRO | B | 106 | 27.204 | 50.544 | 50.281 | 1.00 | 64.12 | C |
| ATOM | 2774 | O | PRO | B | 106 | 26.922 | 49.337 | 50.383 | 1.00 | 63.52 | O |
| ATOM | 2775 | N | ASN | B | 107 | 26.330 | 51.513 | 50.535 | 1.00 | 65.99 | N |
| ATOM | 2776 | CA | ASN | B | 107 | 24.932 | 51.182 | 50.813 | 1.00 | 64.19 | C |
| ATOM | 2777 | CB | ASN | B | 107 | 24.138 | 52.353 | 51.412 | 1.00 | 62.06 | C |
| ATOM | 2778 | CG | ASN | B | 107 | 24.020 | 53.528 | 50.459 | 1.00 | 65.95 | C |
| ATOM | 2779 | OD1 | ASN | B | 107 | 24.993 | 53.930 | 49.814 | 1.00 | 97.12 | O |
| ATOM | 2780 | ND2 | ASN | B | 107 | 22.847 | 54.134 | 50.429 | 1.00 | 59.12 | N |
| ATOM | 2781 | C | ASN | B | 107 | 24.350 | 50.837 | 49.479 | 1.00 | 62.84 | C |
| ATOM | 2782 | O | ASN | B | 107 | 24.758 | 51.371 | 48.463 | 1.00 | 74.25 | O |
| ATOM | 2783 | N | GLN | B | 108 | 23.456 | 49.864 | 49.439 | 1.00 | 56.75 | N |
| ATOM | 2784 | CA | GLN | B | 108 | 22.774 | 49.633 | 48.174 | 1.00 | 51.22 | C |
| ATOM | 2785 | CB | GLN | B | 108 | 21.737 | 48.518 | 48.376 | 1.00 | 59.46 | C |
| ATOM | 2786 | CG | GLN | B | 108 | 22.123 | 47.526 | 49.498 | 1.00 | 71.26 | C |
| ATOM | 2787 | CD | GLN | B | 108 | 23.146 | 46.518 | 49.020 | 1.00 | 73.58 | C |
| ATOM | 2788 | OE1 | GLN | B | 108 | 24.295 | 46.888 | 48.822 | 1.00 | 76.35 | O |
| ATOM | 2789 | NE2 | GLN | B | 108 | 22.709 | 45.284 | 48.735 | 1.00 | 76.60 | N |
| ATOM | 2790 | C | GLN | B | 108 | 22.074 | 50.944 | 47.766 | 1.00 | 52.36 | C |
| ATOM | 2791 | O | GLN | B | 108 | 22.247 | 52.005 | 48.389 | 1.00 | 54.37 | O |
| ATOM | 2792 | N | GLU | B | 109 | 21.177 | 50.855 | 46.789 | 1.00 | 46.03 | N |
| ATOM | 2793 | CA | GLU | B | 109 | 20.556 | 52.072 | 46.251 | 1.00 | 41.24 | C |
| ATOM | 2794 | CB | GLU | B | 109 | 20.300 | 53.144 | 47.329 | 1.00 | 35.59 | C |
| ATOM | 2795 | CG | GLU | B | 109 | 19.709 | 54.433 | 46.736 | 1.00 | 71.84 | C |
| ATOM | 2796 | CD | GLU | B | 109 | 19.460 | 55.543 | 47.766 | 1.00 | 101.72 | C |
| ATOM | 2797 | OE1 | GLU | B | 109 | 19.380 | 55.209 | 48.978 | 1.00 | 105.91 | O |
| ATOM | 2798 | OE2 | GLU | B | 109 | 19.330 | 56.747 | 47.372 | 1.00 | 86.68 | O |
| ATOM | 2799 | C | GLU | B | 109 | 21.510 | 52.644 | 45.245 | 1.00 | 35.32 | C |
| ATOM | 2800 | O | GLU | B | 109 | 22.705 | 52.699 | 45.467 | 1.00 | 42.85 | O |
| ATOM | 2801 | N | LYS | B | 110 | 21.022 | 52.968 | 44.075 | 1.00 | 28.66 | N |
| ATOM | 2802 | CA | LYS | B | 110 | 21.995 | 53.138 | 43.008 | 1.00 | 35.82 | C |
| ATOM | 2803 | CB | LYS | B | 110 | 22.814 | 54.405 | 43.236 | 1.00 | 29.50 | C |
| ATOM | 2804 | CG | LYS | B | 110 | 23.014 | 55.201 | 41.942 | 1.00 | 40.10 | C |
| ATOM | 2805 | CD | LYS | B | 110 | 24.439 | 55.772 | 41.889 | 1.00 | 55.09 | C |
| ATOM | 2806 | CE | LYS | B | 110 | 25.130 | 55.486 | 40.573 | 1.00 | 57.60 | C |
| ATOM | 2807 | NZ | LYS | B | 110 | 26.511 | 54.985 | 40.843 | 1.00 | 64.19 | N |
| ATOM | 2808 | C | LYS | B | 110 | 22.912 | 51.912 | 42.862 | 1.00 | 34.33 | C |
| ATOM | 2809 | O | LYS | B | 110 | 24.122 | 52.015 | 42.962 | 1.00 | 37.33 | O |
| ATOM | 2810 | N | VAL | B | 111 | 22.338 | 50.823 | 42.363 | 1.00 | 34.29 | N |
| ATOM | 2811 | CA | VAL | B | 111 | 23.121 | 49.721 | 41.837 | 1.00 | 34.88 | C |
| ATOM | 2812 | CB | VAL | B | 111 | 22.558 | 48.430 | 42.402 | 1.00 | 32.28 | C |
| ATOM | 2813 | CG1 | VAL | B | 111 | 23.438 | 47.240 | 42.052 | 1.00 | 21.54 | C |
| ATOM | 2814 | CG2 | VAL | B | 111 | 22.345 | 48.611 | 43.893 | 1.00 | 34.67 | C |
| ATOM | 2815 | C | VAL | B | 111 | 23.185 | 49.660 | 40.295 | 1.00 | 34.97 | C |
| ATOM | 2816 | O | VAL | B | 111 | 22.179 | 49.582 | 39.612 | 1.00 | 28.36 | O |
| ATOM | 2817 | N | SER | B | 112 | 24.393 | 49.709 | 39.747 | 1.00 | 39.84 | N |
| ATOM | 2818 | CA | SER | B | 112 | 24.574 | 49.733 | 38.298 | 1.00 | 39.76 | C |
| ATOM | 2819 | CB | SER | B | 112 | 25.585 | 50.816 | 37.919 | 1.00 | 38.00 | C |
| ATOM | 2820 | OG | SER | B | 112 | 25.394 | 51.971 | 38.738 | 1.00 | 37.85 | O |
| ATOM | 2821 | C | SER | B | 112 | 25.052 | 48.386 | 37.737 | 1.00 | 42.73 | C |
| ATOM | 2822 | O | SER | B | 112 | 25.856 | 47.694 | 38.353 | 1.00 | 39.71 | O |
| ATOM | 2823 | N | ASP | B | 113 | 24.533 | 48.013 | 36.572 | 1.00 | 42.77 | N |
| ATOM | 2824 | CA | ASP | B | 113 | 24.691 | 46.664 | 36.052 | 1.00 | 37.73 | C |
| ATOM | 2825 | CB | ASP | B | 113 | 23.397 | 45.865 | 36.227 | 1.00 | 40.88 | C |
| ATOM | 2826 | CG | ASP | B | 113 | 23.619 | 44.375 | 36.003 | 1.00 | 67.98 | C |
| ATOM | 2827 | OD1 | ASP | B | 113 | 23.053 | 43.537 | 36.764 | 1.00 | 76.80 | O |
| ATOM | 2828 | OD2 | ASP | B | 113 | 24.445 | 43.966 | 35.149 | 1.00 | 87.89 | O |
| ATOM | 2829 | C | ASP | B | 113 | 25.018 | 46.729 | 34.583 | 1.00 | 30.20 | C |
| ATOM | 2830 | O | ASP | B | 113 | 24.120 | 46.775 | 33.760 | 1.00 | 42.66 | O |
| ATOM | 2831 | N | TYR | B | 114 | 26.299 | 46.782 | 34.252 | 1.00 | 25.36 | N |
| ATOM | 2832 | CA | TYR | B | 114 | 26.732 | 47.055 | 32.882 | 1.00 | 28.69 | C |
| ATOM | 2833 | CB | TYR | B | 114 | 27.907 | 48.029 | 32.851 | 1.00 | 25.90 | C |
| ATOM | 2834 | CG | TYR | B | 114 | 27.552 | 49.458 | 33.188 | 1.00 | 26.76 | C |
| ATOM | 2835 | CD1 | TYR | B | 114 | 27.312 | 50.375 | 32.193 | 1.00 | 24.15 | C |
| ATOM | 2836 | CE1 | TYR | B | 114 | 26.946 | 51.659 | 32.492 | 1.00 | 11.50 | C |
| ATOM | 2837 | CZ | TYR | B | 114 | 26.816 | 52.042 | 33.822 | 1.00 | 33.42 | C |
| ATOM | 2838 | OH | TYR | B | 114 | 26.547 | 53.346 | 34.198 | 1.00 | 33.61 | O |
| ATOM | 2839 | CE2 | TYR | B | 114 | 27.069 | 51.153 | 34.820 | 1.00 | 22.37 | C |
| ATOM | 2840 | CD2 | TYR | B | 114 | 27.447 | 49.881 | 34.501 | 1.00 | 27.00 | C |
| ATOM | 2841 | C | TYR | B | 114 | 27.135 | 45.775 | 32.165 | 1.00 | 27.73 | C |
| ATOM | 2842 | O | TYR | B | 114 | 27.427 | 44.779 | 32.795 | 1.00 | 29.84 | O |
| ATOM | 2843 | N | GLU | B | 115 | 27.047 | 45.767 | 30.844 | 1.00 | 25.84 | N |
| ATOM | 2844 | CA | GLU | B | 115 | 27.172 | 44.521 | 30.098 | 1.00 | 23.42 | C |
| ATOM | 2845 | CB | GLU | B | 115 | 25.792 | 44.039 | 29.671 | 1.00 | 23.62 | C |
| ATOM | 2846 | CG | GLU | B | 115 | 25.764 | 42.583 | 29.236 | 1.00 | 55.59 | C |
| ATOM | 2847 | CD | GLU | B | 115 | 24.468 | 41.903 | 29.649 | 1.00 | 85.83 | C |
| ATOM | 2848 | OE1 | GLU | B | 115 | 23.388 | 42.313 | 29.161 | 1.00 | 79.11 | O |
| ATOM | 2849 | OE2 | GLU | B | 115 | 24.524 | 40.964 | 30.478 | 1.00 | 103.61 | O |
| ATOM | 2850 | C | GLU | B | 115 | 27.965 | 44.804 | 28.841 | 1.00 | 31.88 | C |
| ATOM | 2851 | O | GLU | B | 115 | 27.566 | 45.623 | 27.986 | 1.00 | 43.46 | O |
| ATOM | 2852 | N | MET | B | 116 | 29.101 | 44.134 | 28.728 | 1.00 | 31.73 | N |
| ATOM | 2853 | CA | MET | B | 116 | 30.052 | 44.488 | 27.682 | 1.00 | 32.07 | C |
| ATOM | 2854 | CB | MET | B | 116 | 31.385 | 44.935 | 28.280 | 1.00 | 35.94 | C |
| ATOM | 2855 | CG | MET | B | 116 | 32.327 | 45.581 | 27.267 | 1.00 | 51.84 | C |
| ATOM | 2856 | SD | MET | B | 116 | 34.018 | 45.748 | 27.890 | 1.00 | 52.10 | S |
| ATOM | 2857 | CE | MET | B | 116 | 34.126 | 44.302 | 28.913 | 1.00 | 61.49 | C |
| ATOM | 2858 | C | MET | B | 116 | 30.287 | 43.320 | 26.761 | 1.00 | 22.54 | C |
| ATOM | 2859 | O | MET | B | 116 | 30.774 | 42.286 | 27.204 | 1.00 | 25.55 | O |
| ATOM | 2860 | N | LYS | B | 117 | 30.004 | 43.508 | 25.472 | 1.00 | 34.77 | N |
| ATOM | 2861 | CA | LYS | B | 117 | 30.279 | 42.469 | 24.478 | 1.00 | 43.30 | C |
| ATOM | 2862 | CB | LYS | B | 117 | 29.766 | 42.918 | 23.106 | 1.00 | 36.02 | C |
| ATOM | 2863 | CG | LYS | B | 117 | 28.394 | 42.357 | 22.765 | 1.00 | 54.02 | C |
| ATOM | 2864 | CD | LYS | B | 117 | 27.327 | 43.424 | 23.005 | 1.00 | 65.35 | C |
| ATOM | 2865 | CE | LYS | B | 117 | 26.629 | 43.791 | 21.697 | 1.00 | 77.40 | C |
| ATOM | 2866 | NZ | LYS | B | 117 | 27.207 | 45.015 | 21.038 | 1.00 | 58.09 | N |
| ATOM | 2867 | C | LYS | B | 117 | 31.772 | 42.095 | 24.415 | 1.00 | 43.06 | C |
| ATOM | 2868 | O | LYS | B | 117 | 32.602 | 42.955 | 24.154 | 1.00 | 56.27 | O |
| ATOM | 2869 | N | LEU | B | 118 | 32.127 | 40.830 | 24.632 | 1.00 | 34.56 | N |
| ATOM | 2870 | CA | LEU | B | 118 | 33.475 | 40.378 | 24.293 | 1.00 | 34.44 | C |
| ATOM | 2871 | CB | LEU | B | 118 | 33.727 | 38.997 | 24.883 | 1.00 | 33.27 | C |
| ATOM | 2872 | CG | LEU | B | 118 | 33.251 | 38.850 | 26.326 | 1.00 | 28.97 | C |
| ATOM | 2873 | CD1 | LEU | B | 118 | 33.614 | 37.438 | 26.783 | 1.00 | 37.08 | C |
| ATOM | 2874 | CD2 | LEU | B | 118 | 33.938 | 39.893 | 27.209 | 1.00 | 50.40 | C |
| ATOM | 2875 | C | LEU | B | 118 | 33.729 | 40.321 | 22.790 | 1.00 | 43.54 | C |
| ATOM | 2876 | O | LEU | B | 118 | 32.819 | 40.552 | 21.976 | 1.00 | 47.17 | O |
| ATOM | 2877 | N | MET | B | 119 | 34.957 | 39.937 | 22.429 | 1.00 | 50.16 | N |
| ATOM | 2878 | CA | MET | B | 119 | 35.326 | 39.733 | 21.027 | 1.00 | 46.05 | C |
| ATOM | 2879 | CB | MET | B | 119 | 35.770 | 41.055 | 20.390 | 1.00 | 55.90 | C |
| ATOM | 2880 | CG | MET | B | 119 | 36.636 | 41.940 | 21.293 | 1.00 | 64.46 | C |
| ATOM | 2881 | SD | MET | B | 119 | 37.962 | 42.699 | 20.320 | 1.00 | 91.31 | S |
| ATOM | 2882 | CE | MET | B | 119 | 37.066 | 44.056 | 19.496 | 1.00 | 51.11 | C |
| ATOM | 2883 | C | MET | B | 119 | 36.389 | 38.655 | 20.874 | 1.00 | 38.44 | C |
| ATOM | 2884 | O | MET | B | 119 | 37.117 | 38.344 | 21.818 | 1.00 | 28.23 | O |
| ATOM | 2885 | N | ASP | B | 120 | 36.465 | 38.066 | 19.684 | 1.00 | 49.38 | N |
| ATOM | 2886 | CA | ASP | B | 120 | 37.463 | 37.023 | 19.440 | 1.00 | 59.71 | C |
| ATOM | 2887 | CB | ASP | B | 120 | 37.052 | 36.116 | 18.276 | 1.00 | 52.97 | C |
| ATOM | 2888 | CG | ASP | B | 120 | 37.296 | 34.651 | 18.573 | 1.00 | 68.88 | C |
| ATOM | 2889 | OD1 | ASP | B | 120 | 38.009 | 34.363 | 19.568 | 1.00 | 59.27 | O |
| ATOM | 2890 | OD2 | ASP | B | 120 | 36.808 | 33.728 | 17.876 | 1.00 | 83.09 | O |
| ATOM | 2891 | C | ASP | B | 120 | 38.841 | 37.635 | 19.191 | 1.00 | 63.90 | C |
| ATOM | 2892 | O | ASP | B | 120 | 38.958 | 38.650 | 18.491 | 1.00 | 60.79 | O |
| ATOM | 2893 | N | LEU | B | 121 | 39.870 | 37.038 | 19.793 | 1.00 | 65.75 | N |
| ATOM | 2894 | CA | LEU | B | 121 | 41.237 | 37.524 | 19.613 | 1.00 | 70.98 | C |
| ATOM | 2895 | CB | LEU | B | 121 | 41.777 | 38.135 | 20.911 | 1.00 | 73.99 | C |
| ATOM | 2896 | CG | LEU | B | 121 | 41.323 | 39.537 | 21.357 | 1.00 | 71.42 | C |
| ATOM | 2897 | CD1 | LEU | B | 121 | 42.104 | 39.982 | 22.595 | 1.00 | 37.13 | C |
| ATOM | 2898 | CD2 | LEU | B | 121 | 41.376 | 40.599 | 20.239 | 1.00 | 63.58 | C |
| ATOM | 2899 | C | LEU | B | 121 | 42.220 | 36.483 | 19.065 | 1.00 | 73.39 | C |
| ATOM | 2900 | O | LEU | B | 121 | 42.343 | 35.372 | 19.586 | 1.00 | 66.68 | O |
| ATOM | 2901 | N | ASP | B | 122 | 42.936 | 36.866 | 18.014 | 1.00 | 81.68 | N |
| ATOM | 2902 | CA | ASP | B | 122 | 43.928 | 35.986 | 17.414 | 1.00 | 88.82 | C |
| ATOM | 2903 | CB | ASP | B | 122 | 44.070 | 36.332 | 15.929 | 1.00 | 92.18 | C |
| ATOM | 2904 | CG | ASP | B | 122 | 44.599 | 35.179 | 15.117 | 1.00 | 91.83 | C |
| ATOM | 2905 | OD1 | ASP | B | 122 | 45.838 | 35.001 | 15.099 | 1.00 | 93.52 | O |
| ATOM | 2906 | OD2 | ASP | B | 122 | 43.844 | 34.385 | 14.512 | 1.00 | 82.11 | O |
| ATOM | 2907 | C | ASP | B | 122 | 45.271 | 36.160 | 18.121 | 1.00 | 92.68 | C |
| ATOM | 2908 | O | ASP | B | 122 | 46.224 | 36.673 | 17.526 | 1.00 | 96.26 | O |
| ATOM | 2909 | N | VAL | B | 123 | 45.329 | 35.792 | 19.401 | 1.00 | 91.39 | N |
| ATOM | 2910 | CA | VAL | B | 123 | 46.366 | 36.317 | 20.295 | 1.00 | 88.71 | C |
| ATOM | 2911 | CB | VAL | B | 123 | 45.960 | 36.263 | 21.785 | 1.00 | 93.61 | C |
| ATOM | 2912 | CG1 | VAL | B | 123 | 46.190 | 34.853 | 22.344 | 1.00 | 79.77 | C |
| ATOM | 2913 | CG2 | VAL | B | 123 | 46.725 | 37.349 | 22.603 | 1.00 | 87.47 | C |
| ATOM | 2914 | C | VAL | B | 123 | 47.665 | 35.549 | 20.146 | 1.00 | 86.34 | C |
| ATOM | 2915 | O | VAL | B | 123 | 47.645 | 34.345 | 19.946 | 1.00 | 86.61 | O |
| ATOM | 2916 | N | GLU | B | 124 | 48.790 | 36.250 | 20.253 | 1.00 | 89.17 | N |
| ATOM | 2917 | CA | GLU | B | 124 | 50.091 | 35.601 | 20.324 | 1.00 | 93.84 | C |
| ATOM | 2918 | CB | GLU | B | 124 | 51.106 | 36.331 | 19.425 | 1.00 | 94.36 | C |
| ATOM | 2919 | CG | GLU | B | 124 | 52.511 | 35.724 | 19.414 | 1.00 | 96.57 | C |
| ATOM | 2920 | CD | GLU | B | 124 | 53.586 | 36.702 | 18.947 | 1.00 | 94.24 | C |
| ATOM | 2921 | OE1 | GLU | B | 124 | 53.811 | 37.718 | 19.646 | 1.00 | 69.24 | O |
| ATOM | 2922 | OE2 | GLU | B | 124 | 54.229 | 36.443 | 17.897 | 1.00 | 87.63 | O |
| ATOM | 2923 | C | GLU | B | 124 | 50.550 | 35.642 | 21.771 | 1.00 | 94.31 | C |
| ATOM | 2924 | O | GLU | B | 124 | 50.599 | 36.724 | 22.383 | 1.00 | 98.95 | O |
| ATOM | 2925 | N | GLN | B | 125 | 50.855 | 34.469 | 22.328 | 1.00 | 92.40 | N |
| ATOM | 2926 | CA | GLN | B | 125 | 51.221 | 34.400 | 23.737 | 1.00 | 91.89 | C |
| ATOM | 2927 | CB | GLN | B | 125 | 50.530 | 33.224 | 24.437 | 1.00 | 94.67 | C |
| ATOM | 2928 | CG | GLN | B | 125 | 49.027 | 33.430 | 24.686 | 1.00 | 96.31 | C |
| ATOM | 2929 | CD | GLN | B | 125 | 48.710 | 34.750 | 25.385 | 1.00 | 103.95 | C |
| ATOM | 2930 | OE1 | GLN | B | 125 | 48.162 | 35.665 | 24.760 | 1.00 | 92.81 | O |
| ATOM | 2931 | NE2 | GLN | B | 128 | 49.049 | 34.849 | 26.678 | 1.00 | 101.83 | N |
| ATOM | 2932 | C | GLN | B | 125 | 52.733 | 34.413 | 23.992 | 1.00 | 89.38 | C |
| ATOM | 2933 | O | GLN | B | 125 | 53.530 | 33.875 | 23.203 | 1.00 | 83.56 | O |
| ATOM | 2934 | N | LEU | B | 126 | 53.109 | 35.086 | 25.083 | 1.00 | 86.50 | N |
| ATOM | 2935 | CA | LEU | B | 126 | 54.506 | 35.430 | 25.372 | 1.00 | 81.12 | C |
| ATOM | 2936 | CB | LEU | B | 126 | 54.678 | 36.942 | 25.501 | 1.00 | 76.66 | C |
| ATOM | 2937 | CG | LEU | B | 126 | 54.987 | 37.622 | 24.168 | 1.00 | 82.40 | C |
| ATOM | 2938 | CD1 | LEU | B | 126 | 56.060 | 38.664 | 24.389 | 1.00 | 87.34 | C |
| ATOM | 2939 | CD2 | LEU | B | 126 | 55.433 | 36.590 | 23.127 | 1.00 | 81.66 | C |
| ATOM | 2940 | C | LEU | B | 126 | 54.958 | 34.803 | 26.672 | 1.00 | 77.57 | C |
| ATOM | 2941 | O | LEU | B | 126 | 54.281 | 34.908 | 27.697 | 1.00 | 78.75 | O |
| ATOM | 2942 | N | GLY | B | 127 | 56.109 | 34.148 | 26.616 | 1.00 | 77.97 | N |
| ATOM | 2943 | CA | GLY | B | 127 | 56.631 | 33.461 | 27.775 | 1.00 | 79.51 | C |
| ATOM | 2944 | C | GLY | B | 127 | 57.908 | 34.140 | 28.209 | 1.00 | 82.25 | C |
| ATOM | 2945 | O | GLY | B | 127 | 58.972 | 33.901 | 27.616 | 1.00 | 85.32 | O |
| ATOM | 2946 | N | ILE | B | 128 | 57.797 | 34.999 | 29.223 | 1.00 | 76.70 | N |
| ATOM | 2947 | CA | ILE | B | 128 | 58.972 | 35.539 | 29.908 | 1.00 | 70.86 | C |
| ATOM | 2948 | CB | ILE | B | 128 | 58.740 | 37.018 | 30.259 | 1.00 | 66.78 | C |
| ATOM | 2949 | CG1 | ILE | B | 128 | 58.072 | 37.696 | 29.079 | 1.00 | 50.11 | C |
| ATOM | 2950 | CD1 | ILE | B | 128 | 56.570 | 37.785 | 29.291 | 1.00 | 73.26 | C |
| ATOM | 2951 | CG2 | ILE | B | 128 | 60.042 | 37.741 | 30.602 | 1.00 | 51.21 | C |
| ATOM | 2952 | C | ILE | B | 128 | 59.359 | 34.725 | 31.146 | 1.00 | 73.95 | C |
| ATOM | 2953 | O | ILE | B | 128 | 58.594 | 34.643 | 32.114 | 1.00 | 75.58 | O |
| ATOM | 2954 | N | PRO | B | 129 | 60.560 | 34.145 | 31.105 | 1.00 | 75.43 | N |
| ATOM | 2955 | CA | PRO | B | 129 | 61.137 | 33.425 | 32.250 | 1.00 | 79.26 | C |
| ATOM | 2956 | CB | PRO | B | 129 | 62.247 | 32.581 | 31.601 | 1.00 | 77.99 | C |
| ATOM | 2957 | CG | PRO | B | 129 | 62.684 | 33.383 | 30.396 | 1.00 | 78.47 | C |
| ATOM | 2958 | CD | PRO | B | 129 | 61.471 | 34.167 | 29.945 | 1.00 | 73.58 | C |
| ATOM | 2959 | C | PRO | B | 129 | 61.716 | 34.373 | 33.312 | 1.00 | 75.28 | C |
| ATOM | 2960 | O | PRO | B | 129 | 62.038 | 35.517 | 32.986 | 1.00 | 70.92 | O |
| ATOM | 2961 | N | GLU | B | 130 | 61.783 | 33.924 | 34.565 | 1.00 | 73.53 | N |
| ATOM | 2962 | CA | GLU | B | 130 | 62.287 | 34.769 | 35.650 | 1.00 | 79.73 | C |
| ATOM | 2963 | CB | GLU | B | 130 | 61.974 | 34.156 | 37.019 | 1.00 | 80.33 | C |
| ATOM | 2964 | CG | GLU | B | 130 | 60.720 | 34.697 | 37.700 | 1.00 | 89.89 | C |
| ATOM | 2965 | CD | GLU | B | 130 | 60.179 | 33.722 | 38.742 | 1.00 | 102.37 | C |
| ATOM | 2966 | OE1 | GLU | B | 130 | 60.947 | 32.838 | 39.199 | 1.00 | 97.53 | O |
| ATOM | 2967 | OE2 | GLU | B | 130 | 58.982 | 33.815 | 39.091 | 1.00 | 104.68 | O |
| ATOM | 2968 | C | GLU | B | 130 | 63.795 | 34.983 | 35.516 | 1.00 | 77.72 | C |
| ATOM | 2969 | O | GLU | B | 130 | 64.546 | 34.032 | 35.273 | 1.00 | 81.31 | O |
| ATOM | 2970 | N | GLN | B | 131 | 64.231 | 36.233 | 35.671 | 1.00 | 69.79 | N |
| ATOM | 2971 | CA | GLN | B | 131 | 65.645 | 36.563 | 35.546 | 1.00 | 64.99 | C |
| ATOM | 2972 | CB | GLN | B | 131 | 65.918 | 37.335 | 34.254 | 1.00 | 65.22 | C |
| ATOM | 2973 | CG | GLN | B | 131 | 65.545 | 36.581 | 32.986 | 1.00 | 69.61 | C |
| ATOM | 2974 | CD | GLN | B | 131 | 65.151 | 37.520 | 31.855 | 1.00 | 82.69 | C |
| ATOM | 2975 | OE1 | GLN | B | 131 | 65.949 | 38.375 | 31.448 | 1.00 | 65.40 | O |
| ATOM | 2976 | NE2 | GLN | B | 131 | 63.928 | 37.362 | 31.341 | 1.00 | 69.47 | N |
| ATOM | 2977 | C | GLN | B | 131 | 66.128 | 37.362 | 36.747 | 1.00 | 64.26 | C |
| ATOM | 2978 | O | GLN | B | 131 | 65.348 | 38.048 | 37.412 | 1.00 | 55.71 | O |
| ATOM | 2979 | N | GLU | B | 132 | 67.414 | 37.215 | 37.048 | 1.00 | 67.96 | N |
| ATOM | 2980 | CA | GLU | B | 132 | 68.044 | 37.974 | 38.126 | 1.00 | 74.27 | C |
| ATOM | 2981 | CB | GLU | B | 132 | 68.954 | 37.071 | 38.972 | 1.00 | 76.01 | C |
| ATOM | 2982 | CG | GLU | B | 132 | 68.245 | 36.308 | 40.089 | 1.00 | 84.64 | C |
| ATOM | 2983 | CD | GLU | B | 132 | 68.910 | 36.502 | 41.446 | 1.00 | 86.81 | C |
| ATOM | 2984 | OE1 | GLU | B | 132 | 69.438 | 35.508 | 42.010 | 1.00 | 72.64 | O |
| ATOM | 2985 | OE2 | GLU | B | 132 | 68.906 | 37.651 | 41.945 | 1.00 | 80.61 | O |
| ATOM | 2986 | C | GLU | B | 132 | 68.829 | 39.166 | 37.563 | 1.00 | 74.05 | C |
| ATOM | 2987 | O | GLU | B | 132 | 69.714 | 39.000 | 36.717 | 1.00 | 79.30 | O |
| ATOM | 2988 | N | TYR | B | 133 | 68.468 | 40.370 | 38.006 | 1.00 | 66.01 | N |
| ATOM | 2989 | CA | TYR | B | 133 | 68.852 | 41.593 | 37.312 | 1.00 | 57.16 | C |
| ATOM | 2990 | CB | TYR | B | 133 | 67.679 | 42.568 | 37.259 | 1.00 | 51.40 | C |
| ATOM | 2991 | CG | TYR | B | 133 | 66.528 | 42.061 | 36.444 | 1.00 | 43.86 | C |
| ATOM | 2992 | CD1 | TYR | B | 133 | 65.399 | 41.565 | 37.064 | 1.00 | 63.99 | C |
| ATOM | 2993 | CE1 | TYR | B | 133 | 64.323 | 41.128 | 36.328 | 1.00 | 53.16 | C |
| ATOM | 2994 | CZ | TYR | B | 133 | 64.369 | 41.202 | 34.949 | 1.00 | 31.62 | C |
| ATOM | 2995 | OH | TYR | B | 133 | 63.314 | 40.743 | 34.194 | 1.00 | 56.69 | O |
| ATOM | 2996 | CE2 | TYR | B | 133 | 65.486 | 41.678 | 34.312 | 1.00 | 39.03 | C |
| ATOM | 2997 | CD2 | TYR | B | 133 | 66.559 | 42.098 | 35.059 | 1.00 | 48.68 | C |
| ATOM | 2998 | C | TYR | B | 133 | 70.033 | 42.239 | 38.009 | 1.00 | 51.40 | C |
| ATOM | 2999 | O | TYR | B | 133 | 70.169 | 42.130 | 39.217 | 1.00 | 48.52 | O |
| ATOM | 3000 | N | SER | B | 134 | 70.876 | 42.913 | 37.232 | 1.00 | 54.37 | N |
| ATOM | 3001 | CA | SER | B | 134 | 72.044 | 43.611 | 37.762 | 1.00 | 55.72 | C |
| ATOM | 3002 | CB | SER | B | 134 | 72.851 | 44.252 | 36.620 | 1.00 | 55.57 | C |
| ATOM | 3003 | OG | SER | B | 134 | 73.064 | 43.344 | 35.564 | 1.00 | 44.11 | O |
| ATOM | 3004 | C | SER | B | 134 | 71.668 | 44.704 | 38.759 | 1.00 | 53.97 | C |
| ATOM | 3005 | O | SER | B | 134 | 72.375 | 44.932 | 39.735 | 1.00 | 42.93 | O |
| ATOM | 3006 | N | CYS | B | 135 | 70.627 | 45.462 | 38.426 | 1.00 | 55.18 | N |
| ATOM | 3007 | CA | CYS | B | 135 | 70.232 | 46.617 | 39.224 | 1.00 | 53.93 | C |
| ATOM | 3008 | CB | CYS | B | 135 | 70.585 | 47.918 | 38.514 | 1.00 | 50.32 | C |
| ATOM | 3009 | SG | CYS | B | 135 | 72.232 | 48.493 | 38.928 | 1.00 | 71.07 | S |
| ATOM | 3010 | C | CYS | B | 135 | 68.734 | 46.569 | 39.474 | 1.00 | 46.02 | C |
| ATOM | 3011 | O | CYS | B | 135 | 67.953 | 48.285 | 38.565 | 1.00 | 43.21 | O |
| ATOM | 3012 | N | VAL | B | 136 | 68.346 | 46.849 | 40.716 | 1.00 | 40.34 | N |
| ATOM | 3013 | CA | VAL | B | 136 | 66.949 | 47.068 | 41.045 | 1.00 | 32.39 | C |
| ATOM | 3014 | CB | VAL | B | 136 | 66.377 | 45.951 | 41.937 | 1.00 | 31.50 | C |
| ATOM | 3015 | CG1 | VAL | B | 136 | 64.895 | 46.153 | 42.148 | 1.00 | 13.38 | C |
| ATOM | 3016 | CG2 | VAL | B | 136 | 66.697 | 44.544 | 41.384 | 1.00 | 15.69 | C |
| ATOM | 3017 | C | VAL | B | 136 | 66.876 | 48.390 | 41.785 | 1.00 | 37.84 | C |
| ATOM | 3018 | O | VAL | B | 136 | 67.307 | 48.493 | 42.929 | 1.00 | 45.41 | O |
| ATOM | 3019 | N | VAL | B | 137 | 66.377 | 49.415 | 41.104 | 1.00 | 36.83 | N |
| ATOM | 3020 | CA | VAL | B | 137 | 66.208 | 50.726 | 41.711 | 1.00 | 43.92 | C |
| ATOM | 3021 | CB | VAL | B | 137 | 66.446 | 51.841 | 40.682 | 1.00 | 44.47 | C |
| ATOM | 3022 | CG1 | VAL | B | 137 | 66.443 | 53.191 | 41.363 | 1.00 | 44.80 | C |
| ATOM | 3023 | CG2 | VAL | B | 137 | 67.768 | 51.624 | 39.983 | 1.00 | 53.27 | C |
| ATOM | 3024 | C | VAL | B | 137 | 64.780 | 50.838 | 42.249 | 1.00 | 50.76 | C |
| ATOM | 3025 | O | VAL | B | 137 | 63.809 | 50.639 | 41.501 | 1.00 | 57.84 | O |
| ATOM | 3026 | N | LYS | B | 138 | 64.649 | 51.132 | 43.542 | 1.00 | 49.40 | N |
| ATOM | 3027 | CA | LYS | B | 138 | 63.355 | 51.509 | 44.110 | 1.00 | 51.75 | C |
| ATOM | 3028 | CB | LYS | B | 138 | 63.199 | 50.908 | 45.511 | 1.00 | 49.85 | C |
| ATOM | 3029 | CG | LYS | B | 138 | 61.859 | 51.219 | 46.174 | 1.00 | 69.70 | C |
| ATOM | 3030 | CD | LYS | B | 138 | 62.046 | 51.858 | 47.576 | 1.00 | 89.74 | C |
| ATOM | 3031 | CE | LYS | B | 138 | 60.743 | 51.854 | 48.412 | 1.00 | 88.04 | C |
| ATOM | 3032 | NZ | LYS | B | 138 | 60.633 | 50.771 | 49.459 | 1.00 | 48.79 | N |
| ATOM | 3033 | C | LYS | B | 138 | 63.234 | 53.037 | 44.149 | 1.00 | 52.77 | C |
| ATOM | 3034 | O | LYS | B | 138 | 64.234 | 53.744 | 44.279 | 1.00 | 58.88 | O |
| ATOM | 3035 | N | MET | B | 139 | 62.031 | 53.559 | 43.935 | 1.00 | 53.53 | N |
| ATOM | 3036 | CA | MET | B | 139 | 61.798 | 55.003 | 44.020 | 1.00 | 43.71 | C |
| ATOM | 3037 | CB | MET | B | 139 | 62.431 | 55.730 | 42.827 | 1.00 | 46.06 | C |
| ATOM | 3038 | CG | MET | B | 139 | 62.153 | 55.035 | 41.513 | 1.00 | 46.36 | C |
| ATOM | 3039 | SD | MET | B | 139 | 62.779 | 55.925 | 40.094 | 1.00 | 76.17 | S |
| ATOM | 3040 | CE | MET | B | 139 | 63.706 | 57.188 | 40.925 | 1.00 | 36.89 | C |
| ATOM | 3041 | C | MET | B | 139 | 60.302 | 55.282 | 44.075 | 1.00 | 38.94 | C |
| ATOM | 3042 | O | MET | B | 139 | 59.478 | 54.479 | 43.635 | 1.00 | 32.73 | O |
| ATOM | 3043 | N | PRO | B | 140 | 59.957 | 56.401 | 44.683 | 1.00 | 37.01 | N |
| ATOM | 3044 | CA | PRO | B | 140 | 58.570 | 56.878 | 44.723 | 1.00 | 44.58 | C |
| ATOM | 3045 | CB | PRO | B | 140 | 58.702 | 58.280 | 45.307 | 1.00 | 42.72 | C |
| ATOM | 3046 | CG | PRO | B | 140 | 59.964 | 58.216 | 46.114 | 1.00 | 38.93 | C |
| ATOM | 3047 | CD | PRO | B | 140 | 60.888 | 57.287 | 45.395 | 1.00 | 42.40 | C |
| ATOM | 3048 | C | PRO | B | 140 | 57.948 | 56.977 | 43.336 | 1.00 | 49.76 | C |
| ATOM | 3049 | O | PRO | B | 140 | 58.611 | 57.427 | 42.396 | 1.00 | 43.13 | O |
| ATOM | 3050 | N | SER | B | 141 | 56.677 | 56.594 | 43.227 | 1.00 | 48.33 | N |
| ATOM | 3051 | CA | SER | B | 141 | 55.995 | 56.576 | 41.945 | 1.00 | 38.79 | C |
| ATOM | 3052 | CB | SER | B | 141 | 54.656 | 55.867 | 42.109 | 1.00 | 34.66 | C |
| ATOM | 3053 | OG | SER | B | 141 | 53.904 | 56.466 | 43.162 | 1.00 | 36.05 | O |
| ATOM | 3054 | C | SER | B | 141 | 55.812 | 58.004 | 41.400 | 1.00 | 35.52 | C |
| ATOM | 3055 | O | SER | B | 141 | 55.861 | 58.217 | 40.189 | 1.00 | 39.45 | O |
| ATOM | 3056 | N | GLY | B | 142 | 55.622 | 58.970 | 42.297 | 1.00 | 26.09 | N |
| ATOM | 3057 | CA | GLY | B | 142 | 55.546 | 60.370 | 41.928 | 1.00 | 22.87 | C |
| ATOM | 3058 | C | GLY | B | 142 | 56.860 | 60.981 | 41.487 | 1.00 | 28.24 | C |
| ATOM | 3059 | O | GLY | B | 142 | 56.867 | 61.976 | 40.788 | 1.00 | 33.95 | O |
| ATOM | 3060 | N | GLU | B | 143 | 57.979 | 60.410 | 41.911 | 1.00 | 39.75 | N |
| ATOM | 3061 | CA | GLU | B | 143 | 59.306 | 60.879 | 41.497 | 1.00 | 35.08 | C |
| ATOM | 3062 | CB | GLU | B | 143 | 60.378 | 60.236 | 42.391 | 1.00 | 39.48 | C |
| ATOM | 3063 | CG | GLU | B | 143 | 61.735 | 60.929 | 42.406 | 1.00 | 62.23 | C |
| ATOM | 3064 | CD | GLU | B | 143 | 61.766 | 62.167 | 43.294 | 1.00 | 90.32 | C |
| ATOM | 3065 | OE1 | GLU | B | 143 | 62.355 | 62.119 | 44.408 | 1.00 | 69.00 | O |
| ATOM | 3066 | OE2 | GLU | B | 143 | 61.209 | 63.203 | 42.854 | 1.00 | 97.77 | O |
| ATOM | 3067 | C | GLU | B | 143 | 59.569 | 60.459 | 40.049 | 1.00 | 33.70 | C |
| ATOM | 3068 | O | GLU | B | 143 | 60.081 | 61.230 | 39.247 | 1.00 | 32.12 | O |
| ATOM | 3069 | N | PHE | B | 144 | 59.313 | 59.195 | 39.748 | 1.00 | 31.21 | N |
| ATOM | 3070 | CA | PHE | B | 144 | 59.703 | 58.635 | 38.470 | 1.00 | 16.36 | C |
| ATOM | 3071 | CB | PHE | B | 144 | 59.552 | 57.116 | 38.501 | 1.00 | 10.33 | C |
| ATOM | 3072 | CG | PHE | B | 144 | 60.011 | 56.455 | 37.265 | 1.00 | 16.51 | C |
| ATOM | 3073 | CD1 | PHE | B | 144 | 61.286 | 56.691 | 36.795 | 1.00 | 27.08 | C |
| ATOM | 3074 | CE1 | PHE | B | 144 | 61.724 | 56.128 | 35.605 | 1.00 | 21.84 | C |
| ATOM | 3075 | CZ | PHE | B | 144 | 60.852 | 55.363 | 34.856 | 1.00 | 45.64 | C |
| ATOM | 3076 | CE2 | PHE | B | 144 | 59.556 | 55.128 | 35.315 | 1.00 | 26.09 | C |
| ATOM | 3077 | CD2 | PHE | B | 144 | 59.137 | 55.700 | 36.490 | 1.00 | 4.43 | C |
| ATOM | 3078 | C | PHE | B | 144 | 58.804 | 59.255 | 37.415 | 1.00 | 19.81 | C |
| ATOM | 3079 | O | PHE | B | 144 | 59.197 | 59.390 | 36.267 | 1.00 | 29.70 | O |
| ATOM | 3080 | N | ALA | B | 145 | 57.600 | 59.669 | 37.793 | 1.00 | 23.18 | N |
| ATOM | 3081 | CA | ALA | B | 145 | 56.681 | 60.223 | 36.801 | 1.00 | 21.46 | C |
| ATOM | 3082 | CB | ALA | B | 145 | 55.282 | 60.364 | 37.361 | 1.00 | 23.70 | C |
| ATOM | 3083 | C | ALA | B | 145 | 57.199 | 61.575 | 36.388 | 1.00 | 26.20 | C |
| ATOM | 3084 | O | ALA | B | 145 | 57.081 | 61.960 | 35.240 | 1.00 | 35.57 | O |
| ATOM | 3085 | N | ARG | B | 146 | 57.716 | 62.334 | 37.343 | 1.00 | 26.68 | N |
| ATOM | 3086 | CA | ARG | B | 146 | 58.170 | 63.687 | 37.043 | 1.00 | 26.46 | C |
| ATOM | 3087 | CB | ARG | B | 146 | 58.341 | 64.492 | 38.341 | 1.00 | 22.16 | C |
| ATOM | 3088 | CG | ARG | B | 146 | 58.659 | 65.949 | 38.163 | 1.00 | 13.56 | C |
| ATOM | 3089 | CD | ARG | B | 146 | 57.413 | 66.828 | 38.075 | 1.00 | 48.54 | C |
| ATOM | 3090 | NE | ARG | B | 146 | 57.531 | 68.093 | 38.804 | 1.00 | 61.23 | N |
| ATOM | 3091 | CZ | ARG | B | 146 | 58.081 | 69.202 | 38.309 | 1.00 | 68.80 | C |
| ATOM | 3092 | NH1 | ARG | B | 146 | 58.586 | 69.202 | 37.081 | 1.00 | 53.69 | N |
| ATOM | 3093 | NH2 | ARG | B | 146 | 58.160 | 70.303 | 39.051 | 1.00 | 76.98 | N |
| ATOM | 3094 | C | ARG | B | 146 | 59.474 | 63.624 | 36.230 | 1.00 | 31.20 | C |
| ATOM | 3095 | O | ARG | B | 146 | 59.691 | 64.433 | 35.322 | 1.00 | 44.19 | O |
| ATOM | 3096 | N | ILE | B | 147 | 60.325 | 62.646 | 36.518 | 1.00 | 14.06 | N |
| ATOM | 3097 | CA | ILE | B | 147 | 61.581 | 62.573 | 35.813 | 1.00 | 11.05 | C |
| ATOM | 3098 | CB | ILE | B | 147 | 62.452 | 61.485 | 36.403 | 1.00 | 12.95 | C |
| ATOM | 3099 | CG1 | ILE | B | 147 | 63.004 | 61.929 | 37.758 | 1.00 | 20.55 | C |
| ATOM | 3100 | CD1 | ILE | B | 147 | 63.520 | 60.791 | 38.627 | 1.00 | 4.56 | C |
| ATOM | 3101 | CG2 | ILE | B | 147 | 63.592 | 61.134 | 35.460 | 1.00 | 19.28 | C |
| ATOM | 3102 | C | ILE | B | 147 | 61.324 | 62.280 | 34.347 | 1.00 | 20.05 | C |
| ATOM | 3103 | O | ILE | B | 147 | 61.898 | 62.903 | 33.456 | 1.00 | 25.65 | O |
| ATOM | 3104 | N | CYS | B | 148 | 60.424 | 61.348 | 34.084 | 1.00 | 24.96 | N |
| ATOM | 3105 | CA | CYS | B | 148 | 60.144 | 60.987 | 32.710 | 1.00 | 19.78 | C |
| ATOM | 3106 | CB | CYS | B | 148 | 59.223 | 59.761 | 32.675 | 1.00 | 26.65 | C |
| ATOM | 3107 | SG | CYS | B | 148 | 59.988 | 58.164 | 33.080 | 1.00 | 35.13 | S |
| ATOM | 3108 | C | CYS | B | 148 | 59.471 | 62.187 | 32.051 | 1.00 | 26.92 | C |
| ATOM | 3109 | O | CYS | B | 148 | 59.603 | 62.361 | 30.843 | 1.00 | 31.59 | O |
| ATOM | 3110 | N | ARG | B | 149 | 58.704 | 62.981 | 32.813 | 1.00 | 29.38 | N |
| ATOM | 3111 | CA | ARG | B | 149 | 57.870 | 64.028 | 32.213 | 1.00 | 25.24 | C |
| ATOM | 3112 | CB | ARG | B | 149 | 56.789 | 64.540 | 33.155 | 1.00 | 27.87 | C |
| ATOM | 3113 | CG | ARG | B | 149 | 55.517 | 64.944 | 32.399 | 1.00 | 38.51 | C |
| ATOM | 3114 | CD | ARG | B | 149 | 54.999 | 66.332 | 32.727 | 1.00 | 30.89 | C |
| ATOM | 3115 | NE | ARG | B | 149 | 55.232 | 66.667 | 34.129 | 1.00 | 73.11 | N |
| ATOM | 3116 | CZ | ARG | B | 149 | 54.268 | 66.987 | 34.982 | 1.00 | 87.23 | C |
| ATOM | 3117 | NH1 | ARG | B | 149 | 53.003 | 67.024 | 34.579 | 1.00 | 76.95 | N |
| ATOM | 3118 | NH2 | ARG | B | 149 | 54.566 | 67.299 | 36.234 | 1.00 | 99.02 | N |
| ATOM | 3119 | C | ARG | B | 149 | 58.722 | 65.189 | 31.850 | 1.00 | 31.46 | C |
| ATOM | 3120 | O | ARG | B | 149 | 58.515 | 65.836 | 30.828 | 1.00 | 45.07 | O |
| ATOM | 3121 | N | ASP | B | 150 | 59.672 | 65.477 | 32.719 | 1.00 | 32.48 | N |
| ATOM | 3122 | CA | ASP | B | 150 | 60.549 | 66.597 | 32.468 | 1.00 | 35.82 | C |
| ATOM | 3123 | CB | ASP | B | 150 | 61.222 | 66.982 | 33.773 | 1.00 | 41.04 | C |
| ATOM | 3124 | CG | ASP | B | 150 | 60.218 | 67.414 | 34.822 | 1.00 | 51.84 | C |
| ATOM | 3125 | OD1 | ASP | B | 150 | 59.045 | 67.629 | 34.460 | 1.00 | 59.01 | O |
| ATOM | 3126 | OD2 | ASP | B | 150 | 60.498 | 67.549 | 36.032 | 1.00 | 63.48 | O |
| ATOM | 3127 | C | ASP | B | 150 | 61.573 | 66.339 | 31.354 | 1.00 | 30.78 | C |
| ATOM | 3128 | O | ASP | B | 150 | 61.890 | 67.227 | 30.566 | 1.00 | 23.77 | O |
| ATOM | 3129 | N | LEU | B | 151 | 62.084 | 65.114 | 31.281 | 1.00 | 23.14 | N |
| ATOM | 3130 | CA | LEU | B | 151 | 63.002 | 64.763 | 30.208 | 1.00 | 20.82 | C |
| ATOM | 3131 | CB | LEU | B | 151 | 63.736 | 63.482 | 30.568 | 1.00 | 15.23 | C |
| ATOM | 3132 | CG | LEU | B | 151 | 64.676 | 63.639 | 31.770 | 1.00 | 15.13 | C |
| ATOM | 3133 | CD1 | LEU | B | 151 | 65.401 | 62.326 | 31.984 | 1.00 | 25.36 | C |
| ATOM | 3134 | CD2 | LEU | B | 151 | 65.673 | 64.785 | 31.583 | 1.00 | 3.89 | C |
| ATOM | 3135 | C | LEU | B | 151 | 62.332 | 64.626 | 28.839 | 1.00 | 28.36 | C |
| ATOM | 3136 | O | LEU | B | 151 | 62.996 | 64.714 | 27.809 | 1.00 | 28.80 | O |
| ATOM | 3137 | N | SER | B | 152 | 61.019 | 64.379 | 28.840 | 1.00 | 37.09 | N |
| ATOM | 3138 | CA | SER | B | 152 | 60.212 | 64.383 | 27.626 | 1.00 | 34.89 | C |
| ATOM | 3139 | CB | SER | B | 152 | 58.752 | 64.108 | 27.961 | 1.00 | 28.65 | C |
| ATOM | 3140 | OG | SER | B | 152 | 58.579 | 62.719 | 28.145 | 1.00 | 56.67 | O |
| ATOM | 3141 | C | SER | B | 152 | 60.303 | 65.713 | 26.903 | 1.00 | 35.71 | C |
| ATOM | 3142 | O | SER | B | 152 | 60.152 | 65.774 | 25.683 | 1.00 | 49.50 | O |
| ATOM | 3143 | N | HIS | B | 153 | 60.473 | 66.780 | 27.670 | 1.00 | 29.85 | N |
| ATOM | 3144 | CA | HIS | B | 153 | 60.509 | 68.105 | 27.101 | 1.00 | 30.23 | C |
| ATOM | 3145 | CB | HIS | B | 153 | 60.207 | 69.142 | 28.174 | 1.00 | 34.52 | C |
| ATOM | 3146 | CG | HIS | B | 153 | 58.767 | 69.174 | 28.593 | 1.00 | 60.20 | C |
| ATOM | 3147 | ND1 | HIS | B | 153 | 57.814 | 68.325 | 28.056 | 1.00 | 51.02 | N |
| ATOM | 3148 | CE1 | HIS | B | 153 | 56.647 | 68.557 | 28.634 | 1.00 | 62.00 | C |
| ATOM | 3149 | NE2 | HIS | B | 153 | 56.796 | 69.568 | 29.481 | 1.00 | 71.64 | N |
| ATOM | 3150 | CD2 | HIS | B | 153 | 58.111 | 69.976 | 29.472 | 1.00 | 58.26 | C |
| ATOM | 3151 | C | HIS | B | 153 | 61.885 | 68.337 | 26.527 | 1.00 | 30.34 | C |
| ATOM | 3152 | O | HIS | B | 153 | 62.067 | 69.247 | 25.717 | 1.00 | 38.08 | O |
| ATOM | 3153 | N | ILE | B | 154 | 62.853 | 67.527 | 26.951 | 1.00 | 28.13 | N |
| ATOM | 3154 | CA | ILE | B | 154 | 64.236 | 67.711 | 26.504 | 1.00 | 29.78 | C |
| ATOM | 3155 | CB | ILE | B | 154 | 65.258 | 67.247 | 27.574 | 1.00 | 35.37 | C |
| ATOM | 3156 | CG1 | ILE | B | 154 | 65.189 | 68.110 | 28.828 | 1.00 | 9.56 | C |
| ATOM | 3157 | CD1 | ILE | B | 154 | 65.449 | 69.534 | 28.546 | 1.00 | 15.41 | C |
| ATOM | 3158 | CG2 | ILE | B | 154 | 66.654 | 67.294 | 27.019 | 1.00 | 17.18 | C |
| ATOM | 3159 | C | ILE | B | 154 | 64.466 | 66.909 | 25.229 | 1.00 | 33.03 | C |
| ATOM | 3160 | O | ILE | B | 154 | 64.715 | 67.475 | 24.181 | 1.00 | 44.20 | O |
| ATOM | 3161 | N | GLY | B | 155 | 64.360 | 65.587 | 25.327 | 1.00 | 34.40 | N |
| ATOM | 3162 | CA | GLY | B | 155 | 64.736 | 64.689 | 24.248 | 1.00 | 29.75 | C |
| ATOM | 3163 | C | GLY | B | 155 | 63.738 | 63.555 | 24.148 | 1.00 | 32.92 | C |
| ATOM | 3164 | O | GLY | B | 155 | 62.708 | 63.605 | 24.814 | 1.00 | 32.84 | O |
| ATOM | 3165 | N | ASP | B | 156 | 63.975 | 62.616 | 23.227 | 1.00 | 36.54 | N |
| ATOM | 3166 | CA | ASP | B | 156 | 63.019 | 61.542 | 22.922 | 1.00 | 31.44 | C |
| ATOM | 3167 | CB | ASP | B | 156 | 62.964 | 61.223 | 21.420 | 1.00 | 26.67 | C |
| ATOM | 3168 | CG | ASP | B | 156 | 61.768 | 61.873 | 20.735 | 1.00 | 49.94 | C |
| ATOM | 3169 | OD1 | ASP | B | 156 | 61.264 | 62.860 | 21.326 | 1.00 | 46.91 | O |
| ATOM | 3170 | OD2 | ASP | B | 156 | 61.264 | 61.475 | 19.644 | 1.00 | 39.34 | O |
| ATOM | 3171 | C | ASP | B | 156 | 63.360 | 60.283 | 23.699 | 1.00 | 19.61 | C |
| ATOM | 3172 | O | ASP | B | 156 | 62.513 | 59.423 | 23.888 | 1.00 | 31.11 | O |
| ATOM | 3173 | N | ALA | B | 157 | 64.619 | 60.157 | 24.083 | 1.00 | 12.29 | N |
| ATOM | 3174 | CA | ALA | B | 157 | 65.136 | 58.935 | 24.694 | 1.00 | 24.20 | C |
| ATOM | 3175 | CB | ALA | B | 157 | 66.073 | 58.189 | 23.728 | 1.00 | 17.33 | C |
| ATOM | 3176 | C | ALA | B | 157 | 65.892 | 59.306 | 25.975 | 1.00 | 34.28 | C |
| ATOM | 3177 | O | ALA | B | 157 | 66.538 | 60.367 | 26.055 | 1.00 | 39.63 | O |
| ATOM | 3178 | N | VAL | B | 158 | 65.824 | 58.420 | 26.965 | 1.00 | 27.39 | N |
| ATOM | 3179 | CA | VAL | B | 158 | 66.516 | 58.646 | 28.220 | 1.00 | 22.99 | C |
| ATOM | 3180 | CB | VAL | B | 158 | 65.556 | 58.575 | 29.414 | 1.00 | 18.46 | C |
| ATOM | 3181 | CG1 | VAL | B | 158 | 64.701 | 57.333 | 29.357 | 1.00 | 30.35 | C |
| ATOM | 3182 | CG2 | VAL | B | 158 | 66.338 | 58.558 | 30.703 | 1.00 | 8.92 | C |
| ATOM | 3183 | C | VAL | B | 158 | 67.666 | 57.655 | 28.416 | 1.00 | 29.65 | C |
| ATOM | 3184 | O | VAL | B | 158 | 67.500 | 56.446 | 28.275 | 1.00 | 30.03 | O |
| ATOM | 3185 | N | VAL | B | 159 | 68.842 | 58.173 | 28.751 | 1.00 | 41.79 | N |
| ATOM | 3186 | CA | VAL | B | 159 | 69.976 | 57.330 | 29.122 | 1.00 | 39.93 | C |
| ATOM | 3187 | CB | VAL | B | 159 | 71.286 | 57.947 | 28.637 | 1.00 | 36.82 | C |
| ATOM | 3188 | CG1 | VAL | B | 159 | 72.436 | 56.940 | 28.775 | 1.00 | 14.35 | C |
| ATOM | 3189 | CG2 | VAL | B | 159 | 71.103 | 58.532 | 27.219 | 1.00 | 28.32 | C |
| ATOM | 3190 | C | VAL | B | 159 | 70.045 | 57.115 | 30.640 | 1.00 | 43.08 | C |
| ATOM | 3191 | O | VAL | B | 159 | 70.434 | 58.014 | 31.390 | 1.00 | 43.51 | O |
| ATOM | 3192 | N | ILE | B | 160 | 69.620 | 55.936 | 31.083 | 1.00 | 33.46 | N |
| ATOM | 3193 | CA | ILE | B | 160 | 69.683 | 55.569 | 32.487 | 1.00 | 29.12 | C |
| ATOM | 3194 | CB | ILE | B | 160 | 68.542 | 54.623 | 32.792 | 1.00 | 20.77 | C |
| ATOM | 3195 | CG1 | ILE | B | 160 | 67.213 | 55.280 | 32.465 | 1.00 | 18.10 | C |
| ATOM | 3196 | CD1 | ILE | B | 160 | 66.023 | 54.434 | 32.883 | 1.00 | 5.48 | C |
| ATOM | 3197 | CG2 | ILE | B | 160 | 68.570 | 54.188 | 34.221 | 1.00 | 18.69 | C |
| ATOM | 3198 | C | ILE | B | 160 | 71.014 | 54.882 | 32.835 | 1.00 | 43.89 | C |
| ATOM | 3199 | O | ILE | B | 160 | 71.323 | 53.801 | 32.322 | 1.00 | 48.88 | O |
| ATOM | 3200 | N | SER | B | 161 | 71.798 | 55.492 | 33.724 | 1.00 | 49.08 | N |
| ATOM | 3201 | CA | SER | B | 161 | 73.052 | 54.879 | 34.159 | 1.00 | 49.22 | C |
| ATOM | 3202 | CB | SER | B | 161 | 74.254 | 55.786 | 33.871 | 1.00 | 60.46 | C |
| ATOM | 3203 | OG | SER | B | 161 | 74.066 | 56.566 | 32.698 | 1.00 | 77.21 | O |
| ATOM | 3204 | C | SER | B | 161 | 72.979 | 54.592 | 35.641 | 1.00 | 44.80 | C |
| ATOM | 3205 | O | SER | B | 161 | 72.371 | 55.342 | 36.384 | 1.00 | 48.02 | O |
| ATOM | 3206 | N | CYS | B | 162 | 73.601 | 53.502 | 36.060 | 1.00 | 36.06 | N |
| ATOM | 3207 | CA | CYS | B | 162 | 73.412 | 52.999 | 37.401 | 1.00 | 42.69 | C |
| ATOM | 3208 | CB | CYS | B | 162 | 72.440 | 51.826 | 37.364 | 1.00 | 30.29 | C |
| ATOM | 3209 | SG | CYS | B | 162 | 71.345 | 51.790 | 38.808 | 1.00 | 89.23 | S |
| ATOM | 3210 | C | CYS | B | 162 | 74.756 | 52.567 | 37.978 | 1.00 | 50.06 | C |
| ATOM | 3211 | O | CYS | B | 162 | 75.549 | 51.980 | 37.259 | 1.00 | 52.26 | O |
| ATOM | 3212 | N | ALA | B | 163 | 75.064 | 52.975 | 39.217 | 1.00 | 58.28 | N |
| ATOM | 3213 | CA | ALA | B | 163 | 76.335 | 52.638 | 39.875 | 1.00 | 66.22 | C |
| ATOM | 3214 | CB | ALA | B | 163 | 77.340 | 53.804 | 39.816 | 1.00 | 61.77 | C |
| ATOM | 3215 | C | ALA | B | 163 | 76.128 | 52.199 | 41.318 | 1.00 | 70.32 | C |
| ATOM | 3216 | O | ALA | B | 163 | 75.155 | 51.486 | 41.617 | 1.00 | 70.84 | O |
| ATOM | 3217 | N | LYS | B | 164 | 77.050 | 52.610 | 42.202 | 1.00 | 76.82 | N |
| ATOM | 3218 | CA | LYS | B | 164 | 76.911 | 52.362 | 43.655 | 1.00 | 82.43 | C |
| ATOM | 3219 | CB | LYS | B | 164 | 78.203 | 51.743 | 44.246 | 1.00 | 83.04 | C |
| ATOM | 3220 | CG | LYS | B | 164 | 78.202 | 51.481 | 45.769 | 1.00 | 70.57 | C |
| ATOM | 3221 | CD | LYS | B | 164 | 77.798 | 50.039 | 46.122 | 1.00 | 86.80 | C |
| ATOM | 3222 | CE | LYS | B | 164 | 76.497 | 50.049 | 46.948 | 1.00 | 90.00 | C |
| ATOM | 3223 | NZ | LYS | B | 164 | 75.977 | 48.691 | 47.285 | 1.00 | 70.04 | N |
| ATOM | 3224 | C | LYS | B | 164 | 76.525 | 53.647 | 44.409 | 1.00 | 81.59 | C |
| ATOM | 3225 | O | LYS | B | 164 | 75.796 | 53.610 | 45.416 | 1.00 | 72.91 | O |
| ATOM | 3226 | N | ASP | B | 165 | 76.974 | 54.775 | 43.856 | 1.00 | 76.39 | N |
| ATOM | 3227 | CA | ASP | B | 165 | 76.545 | 56.110 | 44.282 | 1.00 | 72.66 | C |
| ATOM | 3228 | CB | ASP | B | 165 | 77.329 | 57.176 | 43.496 | 1.00 | 70.96 | C |
| ATOM | 3229 | CG | ASP | B | 165 | 78.781 | 56.765 | 43.246 | 1.00 | 72.09 | C |
| ATOM | 3230 | OD1 | ASP | B | 165 | 79.000 | 55.661 | 42.682 | 1.00 | 64.12 | O |
| ATOM | 3231 | OD2 | ASP | B | 165 | 79.765 | 57.423 | 43.677 | 1.00 | 70.01 | O |
| ATOM | 3232 | C | ASP | B | 165 | 75.027 | 56.337 | 44.168 | 1.00 | 65.44 | C |
| ATOM | 3233 | O | ASP | B | 165 | 74.323 | 56.438 | 45.191 | 1.00 | 64.69 | O |
| ATOM | 3234 | N | GLY | B | 166 | 74.532 | 56.400 | 42.925 | 1.00 | 55.96 | N |
| ATOM | 3235 | CA | GLY | B | 166 | 73.091 | 56.227 | 42.676 | 1.00 | 44.76 | C |
| ATOM | 3236 | C | GLY | B | 166 | 72.844 | 56.168 | 41.178 | 1.00 | 44.37 | C |
| ATOM | 3237 | O | GLY | B | 166 | 73.721 | 55.737 | 40.409 | 1.00 | 33.49 | O |
| ATOM | 3238 | N | VAL | B | 167 | 71.646 | 56.592 | 40.771 | 1.00 | 33.31 | N |
| ATOM | 3239 | CA | VAL | B | 167 | 71.196 | 56.461 | 39.396 | 1.00 | 22.71 | C |
| ATOM | 3240 | CB | VAL | B | 167 | 69.841 | 55.737 | 39.324 | 1.00 | 23.19 | C |
| ATOM | 3241 | CG1 | VAL | B | 167 | 68.929 | 56.235 | 40.396 | 1.00 | 31.98 | C |
| ATOM | 3242 | CG2 | VAL | B | 167 | 69.190 | 55.908 | 37.956 | 1.00 | 42.26 | C |
| ATOM | 3243 | C | VAL | B | 167 | 71.112 | 57.822 | 38.743 | 1.00 | 14.77 | C |
| ATOM | 3244 | O | VAL | B | 167 | 70.537 | 58.732 | 39.298 | 1.00 | 18.13 | O |
| ATOM | 3245 | N | LYS | B | 168 | 71.647 | 57.962 | 37.539 | 1.00 | 24.15 | N |
| ATOM | 3246 | CA | LYS | B | 168 | 71.496 | 59.195 | 36.759 | 1.00 | 25.97 | C |
| ATOM | 3247 | CB | LYS | B | 168 | 72.882 | 59.762 | 36.466 | 1.00 | 24.45 | C |
| ATOM | 3248 | CG | LYS | B | 168 | 72.940 | 60.962 | 35.524 | 1.00 | 28.45 | C |
| ATOM | 3249 | CD | LYS | B | 168 | 74.388 | 61.481 | 35.404 | 1.00 | 45.43 | C |
| ATOM | 3250 | CE | LYS | B | 168 | 75.132 | 60.904 | 34.193 | 1.00 | 57.06 | C |
| ATOM | 3251 | NZ | LYS | B | 168 | 76.601 | 60.765 | 34.464 | 1.00 | 53.27 | N |
| ATOM | 3252 | C | LYS | B | 168 | 70.696 | 58.999 | 35.453 | 1.00 | 28.29 | C |
| ATOM | 3253 | O | LYS | B | 168 | 70.983 | 58.124 | 34.635 | 1.00 | 28.89 | O |
| ATOM | 3254 | N | PHE | B | 169 | 69.706 | 59.860 | 35.258 | 1.00 | 19.71 | N |
| ATOM | 3255 | CA | PHE | B | 169 | 68.973 | 59.963 | 34.006 | 1.00 | 11.51 | C |
| ATOM | 3256 | CB | PHE | B | 169 | 67.516 | 60.124 | 34.339 | 1.00 | 11.56 | C |
| ATOM | 3257 | CG | PHE | B | 169 | 66.999 | 59.071 | 35.260 | 1.00 | 16.36 | C |
| ATOM | 3258 | CD1 | PHE | B | 169 | 66.990 | 59.253 | 36.644 | 1.00 | 17.44 | C |
| ATOM | 3259 | CE1 | PHE | B | 169 | 66.439 | 58.296 | 37.473 | 1.00 | 20.61 | C |
| ATOM | 3260 | CZ | PHE | B | 169 | 65.890 | 57.130 | 36.925 | 1.00 | 16.56 | C |
| ATOM | 3261 | CE2 | PHE | B | 169 | 65.870 | 56.961 | 35.546 | 1.00 | 30.06 | C |
| ATOM | 3262 | CD2 | PHE | B | 169 | 66.403 | 57.944 | 34.726 | 1.00 | 24.60 | C |
| ATOM | 3263 | C | PHE | B | 169 | 69.393 | 61.153 | 33.151 | 1.00 | 15.85 | C |
| ATOM | 3264 | O | PHE | B | 169 | 69.581 | 62.278 | 33.632 | 1.00 | 25.38 | O |
| ATOM | 3265 | N | SER | B | 170 | 69.572 | 60.905 | 31.865 | 1.00 | 15.45 | N |
| ATOM | 3266 | CA | SER | B | 170 | 70.070 | 61.971 | 31.001 | 1.00 | 19.13 | C |
| ATOM | 3267 | CB | SER | B | 170 | 71.565 | 61.775 | 30.684 | 1.00 | 14.33 | C |
| ATOM | 3268 | OG | SER | B | 170 | 72.284 | 61.212 | 31.790 | 1.00 | 31.04 | O |
| ATOM | 3269 | C | SER | B | 170 | 69.218 | 61.974 | 29.736 | 1.00 | 18.47 | C |
| ATOM | 3270 | O | SER | B | 170 | 68.772 | 60.919 | 29.298 | 1.00 | 26.38 | O |
| ATOM | 3271 | N | ALA | B | 171 | 68.959 | 63.156 | 29.185 | 1.00 | 18.97 | N |
| ATOM | 3272 | CA | ALA | B | 171 | 68.381 | 63.305 | 27.839 | 1.00 | 26.70 | C |
| ATOM | 3273 | CB | ALA | B | 171 | 66.899 | 63.467 | 27.904 | 1.00 | 15.49 | C |
| ATOM | 3274 | C | ALA | B | 171 | 68.953 | 64.493 | 27.072 | 1.00 | 37.62 | C |
| ATOM | 3275 | O | ALA | B | 171 | 69.425 | 65.462 | 27.678 | 1.00 | 35.13 | O |
| ATOM | 3276 | N | SER | B | 172 | 68.842 | 64.451 | 25.746 | 1.00 | 38.47 | N |
| ATOM | 3277 | CA | SER | B | 172 | 69.389 | 65.522 | 24.921 | 1.00 | 37.15 | C |
| ATOM | 3278 | CB | SER | B | 172 | 70.772 | 65.148 | 24.409 | 1.00 | 45.09 | C |
| ATOM | 3279 | OG | SER | B | 172 | 71.578 | 64.603 | 25.456 | 1.00 | 62.03 | O |
| ATOM | 3280 | C | SER | B | 172 | 68.477 | 65.762 | 23.747 | 1.00 | 41.50 | C |
| ATOM | 3281 | O | SER | B | 172 | 67.897 | 64.829 | 23.208 | 1.00 | 54.95 | O |
| ATOM | 3282 | N | GLY | B | 173 | 68.308 | 67.018 | 23.378 | 1.00 | 36.65 | N |
| ATOM | 3283 | CA | GLY | B | 173 | 67.331 | 67.362 | 22.375 | 1.00 | 35.30 | C |
| ATOM | 3284 | C | GLY | B | 173 | 67.671 | 68.691 | 21.745 | 1.00 | 40.67 | C |
| ATOM | 3285 | O | GLY | B | 173 | 68.833 | 68.967 | 21.466 | 1.00 | 51.14 | O |
| ATOM | 3286 | N | GLU | B | 174 | 66.643 | 69.480 | 21.452 | 1.00 | 48.41 | N |
| ATOM | 3287 | CA | GLU | B | 174 | 66.791 | 70.624 | 20.561 | 1.00 | 50.04 | C |
| ATOM | 3288 | CB | GLU | B | 174 | 65.485 | 70.901 | 19.797 | 1.00 | 59.21 | C |
| ATOM | 3289 | CG | GLU | B | 174 | 65.593 | 71.933 | 18.678 | 1.00 | 64.93 | C |
| ATOM | 3290 | CD | GLU | B | 174 | 66.524 | 71.490 | 17.565 | 1.00 | 91.66 | C |
| ATOM | 3291 | OE1 | GLU | B | 174 | 67.746 | 71.782 | 17.627 | 1.00 | 102.26 | O |
| ATOM | 3292 | OE2 | GLU | B | 174 | 66.030 | 70.826 | 16.631 | 1.00 | 105.52 | O |
| ATOM | 3293 | C | GLU | B | 174 | 67.238 | 71.849 | 21.361 | 1.00 | 44.99 | C |
| ATOM | 3294 | O | GLU | B | 174 | 68.212 | 72.519 | 20.990 | 1.00 | 28.42 | O |
| ATOM | 3295 | N | LEU | B | 175 | 66.575 | 72.088 | 22.493 | 1.00 | 45.85 | N |
| ATOM | 3296 | CA | LEU | B | 175 | 66.876 | 73.264 | 23.315 | 1.00 | 49.14 | C |
| ATOM | 3297 | CB | LEU | B | 175 | 65.647 | 73.731 | 24.104 | 1.00 | 46.15 | C |
| ATOM | 3298 | CG | LEU | B | 175 | 64.811 | 72.677 | 24.829 | 1.00 | 50.56 | C |
| ATOM | 3299 | CD1 | LEU | B | 175 | 65.135 | 72.638 | 26.333 | 1.00 | 43.95 | C |
| ATOM | 3300 | CD2 | LEU | B | 175 | 63.329 | 72.954 | 24.615 | 1.00 | 36.92 | C |
| ATOM | 3301 | C | LEU | B | 175 | 68.102 | 73.150 | 24.238 | 1.00 | 49.11 | C |
| ATOM | 3302 | O | LEU | B | 175 | 68.596 | 74.163 | 24.760 | 1.00 | 38.56 | O |
| ATOM | 3303 | N | GLY | B | 176 | 68.634 | 71.933 | 24.368 | 1.00 | 48.13 | N |
| ATOM | 3304 | CA | GLY | B | 176 | 69.894 | 71.729 | 25.077 | 1.00 | 46.79 | C |
| ATOM | 3305 | C | GLY | B | 176 | 70.043 | 70.293 | 25.537 | 1.00 | 45.96 | C |
| ATOM | 3306 | O | GLY | B | 176 | 69.616 | 69.374 | 24.830 | 1.00 | 56.96 | O |
| ATOM | 3307 | N | ASN | B | 177 | 70.567 | 70.093 | 26.742 | 1.00 | 35.18 | N |
| ATOM | 3308 | CA | ASN | B | 177 | 70.419 | 68.791 | 27.406 | 1.00 | 41.15 | C |
| ATOM | 3309 | CB | ASN | B | 177 | 71.497 | 67.812 | 26.950 | 1.00 | 40.26 | C |
| ATOM | 3310 | CG | ASN | B | 177 | 72.835 | 68.317 | 27.249 | 1.00 | 38.49 | C |
| ATOM | 3311 | OD1 | ASN | B | 177 | 73.251 | 68.266 | 28.408 | 1.00 | 50.97 | O |
| ATOM | 3312 | ND2 | ASN | B | 177 | 73.378 | 69.079 | 26.307 | 1.00 | 35.95 | N |
| ATOM | 3313 | C | ASN | B | 177 | 70.376 | 68.809 | 28.934 | 1.00 | 42.86 | C |
| ATOM | 3314 | O | ASN | B | 177 | 70.705 | 69.809 | 29.567 | 1.00 | 45.02 | O |
| ATOM | 3315 | N | GLY | B | 178 | 69.893 | 67.715 | 29.520 | 1.00 | 34.13 | N |
| ATOM | 3316 | CA | GLY | B | 178 | 69.482 | 67.773 | 30.903 | 1.00 | 24.40 | C |
| ATOM | 3317 | C | GLY | B | 178 | 69.981 | 66.522 | 31.573 | 1.00 | 27.90 | C |
| ATOM | 3318 | O | GLY | B | 178 | 70.088 | 65.493 | 30.931 | 1.00 | 23.24 | O |
| ATOM | 3319 | N | ASN | B | 179 | 70.180 | 66.606 | 32.883 | 1.00 | 33.23 | N |
| ATOM | 3320 | CA | ASN | B | 179 | 70.559 | 65.476 | 33.720 | 1.00 | 41.47 | C |
| ATOM | 3321 | CB | ASN | B | 179 | 72.056 | 65.526 | 33.992 | 1.00 | 39.87 | C |
| ATOM | 3322 | CG | ASN | B | 179 | 72.815 | 64.747 | 32.997 | 1.00 | 53.35 | C |
| ATOM | 3323 | OD1 | ASN | B | 179 | 73.162 | 63.593 | 33.262 | 1.00 | 71.84 | O |
| ATOM | 3324 | ND2 | ASN | B | 179 | 72.985 | 65.315 | 31.791 | 1.00 | 67.81 | N |
| ATOM | 3325 | C | ASN | B | 179 | 69.840 | 65.479 | 35.070 | 1.00 | 42.95 | C |
| ATOM | 3326 | O | ASN | B | 179 | 69.863 | 66.475 | 35.791 | 1.00 | 48.31 | O |
| ATOM | 3327 | N | ILE | B | 180 | 69.411 | 64.303 | 35.508 | 1.00 | 36.76 | N |
| ATOM | 3328 | CA | ILE | B | 180 | 68.920 | 64.145 | 36.866 | 1.00 | 28.83 | C |
| ATOM | 3329 | CB | ILE | B | 180 | 67.464 | 63.802 | 36.794 | 1.00 | 23.59 | C |
| ATOM | 3330 | CG1 | ILE | B | 180 | 66.719 | 64.991 | 36.207 | 1.00 | 39.24 | C |
| ATOM | 3331 | CD1 | ILE | B | 180 | 65.931 | 64.669 | 34.950 | 1.00 | 46.29 | C |
| ATOM | 3332 | CG2 | ILE | B | 180 | 66.932 | 63.506 | 38.160 | 1.00 | 31.04 | C |
| ATOM | 3333 | C | ILE | B | 180 | 69.629 | 63.069 | 37.667 | 1.00 | 34.08 | C |
| ATOM | 3334 | O | ILE | B | 180 | 69.399 | 61.886 | 37.460 | 1.00 | 38.86 | O |
| ATOM | 3335 | N | LYS | B | 181 | 70.472 | 63.479 | 38.610 | 1.00 | 40.55 | N |
| ATOM | 3336 | CA | LYS | B | 181 | 71.298 | 62.532 | 39.351 | 1.00 | 38.62 | C |
| ATOM | 3337 | CB | LYS | B | 181 | 72.725 | 63.088 | 39.551 | 1.00 | 33.64 | C |
| ATOM | 3338 | CG | LYS | B | 181 | 73.666 | 62.160 | 40.311 | 1.00 | 53.76 | C |
| ATOM | 3339 | CD | LYS | B | 181 | 74.713 | 61.470 | 39.417 | 1.00 | 79.03 | C |
| ATOM | 3340 | CE | LYS | B | 181 | 75.334 | 60.225 | 40.087 | 1.00 | 79.77 | C |
| ATOM | 3341 | NZ | LYS | B | 181 | 75.865 | 59.179 | 39.153 | 1.00 | 72.63 | N |
| ATOM | 3342 | C | LYS | B | 181 | 70.583 | 62.340 | 40.668 | 1.00 | 41.54 | C |
| ATOM | 3343 | O | LYS | B | 181 | 70.400 | 63.292 | 41.438 | 1.00 | 46.74 | O |
| ATOM | 3344 | N | LEU | B | 182 | 70.112 | 61.127 | 40.902 | 1.00 | 39.97 | N |
| ATOM | 3345 | CA | LEU | B | 182 | 69.431 | 60.860 | 42.150 | 1.00 | 38.08 | C |
| ATOM | 3346 | CB | LEU | B | 182 | 68.453 | 59.723 | 41.997 | 1.00 | 40.22 | C |
| ATOM | 3347 | CG | LEU | B | 182 | 67.004 | 60.219 | 41.877 | 1.00 | 25.89 | C |
| ATOM | 3348 | CD1 | LEU | B | 182 | 66.672 | 60.474 | 40.483 | 1.00 | 26.77 | C |
| ATOM | 3349 | CD2 | LEU | B | 182 | 66.061 | 59.157 | 42.368 | 1.00 | 37.06 | C |
| ATOM | 3350 | C | LEU | B | 182 | 70.360 | 60.663 | 43.343 | 1.00 | 47.37 | C |
| ATOM | 3351 | O | LEU | B | 182 | 70.729 | 61.636 | 44.010 | 1.00 | 58.07 | O |
| ATOM | 3352 | N | SER | B | 183 | 70.730 | 59.432 | 43.678 | 1.00 | 51.56 | N |
| ATOM | 3353 | CA | SER | B | 183 | 71.635 | 59.284 | 44.840 | 1.00 | 60.60 | C |
| ATOM | 3354 | CB | SER | B | 183 | 72.867 | 60.220 | 44.727 | 1.00 | 60.80 | C |
| ATOM | 3355 | OG | SER | B | 183 | 72.878 | 61.240 | 45.736 | 1.00 | 61.43 | O |
| ATOM | 3356 | C | SER | B | 183 | 71.015 | 59.384 | 46.251 | 1.00 | 60.14 | C |
| ATOM | 3357 | O | SER | B | 183 | 69.933 | 59.930 | 46.440 | 1.00 | 48.84 | O |
| ATOM | 3358 | N | GLN | B | 184 | 71.676 | 58.753 | 47.219 | 1.00 | 69.88 | N |
| ATOM | 3359 | CA | GLN | B | 184 | 71.096 | 58.523 | 48.536 | 1.00 | 67.38 | C |
| ATOM | 3360 | CB | GLN | B | 184 | 71.336 | 57.076 | 48.943 | 1.00 | 70.47 | C |
| ATOM | 3361 | CG | GLN | B | 184 | 71.087 | 56.842 | 50.413 | 1.00 | 85.16 | C |
| ATOM | 3362 | CD | GLN | B | 184 | 69.886 | 55.940 | 50.663 | 1.00 | 95.03 | C |
| ATOM | 3363 | OE1 | GLN | B | 184 | 69.945 | 54.732 | 50.440 | 1.00 | 88.45 | O |
| ATOM | 3364 | NE2 | GLN | B | 184 | 68.789 | 56.530 | 51.118 | 1.00 | 88.67 | N |
| ATOM | 3365 | C | GLN | B | 184 | 71.826 | 59.402 | 49.519 | 1.00 | 67.22 | C |
| ATOM | 3366 | O | GLN | B | 184 | 73.031 | 59.241 | 49.728 | 1.00 | 64.92 | O |
| ATOM | 3367 | N | THR | B | 185 | 71.115 | 60.369 | 50.083 | 1.00 | 78.96 | N |
| ATOM | 3368 | CA | THR | B | 185 | 71.746 | 61.361 | 50.940 | 1.00 | 87.01 | C |
| ATOM | 3369 | CB | THR | B | 185 | 71.307 | 62.746 | 50.515 | 1.00 | 85.03 | C |
| ATOM | 3370 | OG1 | THR | B | 185 | 72.082 | 63.716 | 51.232 | 1.00 | 73.38 | O |
| ATOM | 3371 | CG2 | THR | B | 185 | 69.912 | 62.989 | 51.028 | 1.00 | 78.30 | C |
| ATOM | 3372 | C | THR | B | 185 | 71.284 | 61.131 | 52.365 | 1.00 | 97.18 | C |
| ATOM | 3373 | O | THR | B | 185 | 70.237 | 60.537 | 52.573 | 1.00 | 101.64 | O |
| ATOM | 3374 | N | SER | B | 186 | 72.049 | 61.609 | 53.343 | 1.00 | 104.59 | N |
| ATOM | 3375 | CA | SER | B | 186 | 71.691 | 61.418 | 54.745 | 1.00 | 109.36 | C |
| ATOM | 3376 | CB | SER | B | 186 | 72.881 | 60.913 | 55.569 | 1.00 | 111.28 | C |
| ATOM | 3377 | OG | SER | B | 186 | 73.657 | 62.021 | 56.049 | 1.00 | 113.09 | O |
| ATOM | 3378 | C | SER | B | 186 | 71.093 | 62.682 | 55.348 | 1.00 | 107.68 | C |
| ATOM | 3379 | O | SER | B | 186 | 71.799 | 63.554 | 55.854 | 1.00 | 107.06 | O |
| ATOM | 3380 | N | ASN | B | 187 | 69.772 | 62.779 | 55.231 | 1.00 | 102.02 | N |
| ATOM | 3381 | CA | ASN | B | 187 | 69.002 | 63.887 | 55.776 | 1.00 | 96.69 | C |
| ATOM | 3382 | CB | ASN | B | 187 | 67.719 | 64.018 | 54.966 | 1.00 | 84.06 | C |
| ATOM | 3383 | CG | ASN | B | 187 | 67.119 | 62.667 | 54.670 | 1.00 | 77.08 | C |
| ATOM | 3384 | OD1 | ASN | B | 187 | 67.549 | 62.018 | 53.711 | 1.00 | 83.93 | O |
| ATOM | 3385 | ND2 | ASN | B | 187 | 66.271 | 62.155 | 55.573 | 1.00 | 72.93 | N |
| ATOM | 3386 | C | ASN | B | 187 | 68.612 | 63.538 | 57.217 | 1.00 | 102.34 | C |
| ATOM | 3387 | O | ASN | B | 187 | 69.428 | 63.005 | 58.000 | 1.00 | 96.97 | O |
| ATOM | 3388 | N | VAL | B | 188 | 67.341 | 63.776 | 57.545 | 1.00 | 113.47 | N |
| ATOM | 3389 | CA | VAL | B | 188 | 66.913 | 63.867 | 58.934 | 1.00 | 119.16 | C |
| ATOM | 3390 | CB | VAL | B | 188 | 67.684 | 64.961 | 59.676 | 1.00 | 114.88 | C |
| ATOM | 3391 | CG1 | VAL | B | 188 | 67.470 | 66.280 | 58.991 | 1.00 | 100.24 | C |
| ATOM | 3392 | CG2 | VAL | B | 188 | 67.213 | 65.068 | 61.110 | 1.00 | 95.33 | C |
| ATOM | 3393 | C | VAL | B | 188 | 65.440 | 64.269 | 58.972 | 1.00 | 132.70 | C |
| ATOM | 3394 | O | VAL | B | 188 | 65.027 | 65.116 | 59.802 | 1.00 | 138.18 | O |
| ATOM | 3395 | N | ASP | B | 189 | 64.650 | 63.686 | 58.064 | 1.00 | 142.22 | N |
| ATOM | 3396 | CA | ASP | B | 189 | 63.203 | 63.899 | 58.085 | 1.00 | 145.74 | C |
| ATOM | 3397 | CB | ASP | B | 189 | 62.729 | 64.565 | 56.759 | 1.00 | 145.21 | C |
| ATOM | 3398 | CG | ASP | B | 189 | 63.530 | 64.082 | 55.543 | 1.00 | 142.38 | C |
| ATOM | 3399 | OD1 | ASP | B | 189 | 63.617 | 64.834 | 54.531 | 1.00 | 133.41 | O |
| ATOM | 3400 | OD2 | ASP | B | 189 | 64.126 | 62.978 | 55.505 | 1.00 | 135.58 | O |
| ATOM | 3401 | C | ASP | B | 189 | 62.439 | 62.576 | 58.325 | 1.00 | 148.00 | C |
| ATOM | 3402 | O | ASP | B | 189 | 62.973 | 61.469 | 58.094 | 1.00 | 147.46 | O |
| ATOM | 3403 | N | LYS | B | 190 | 61.188 | 62.704 | 58.785 | 1.00 | 149.38 | N |
| ATOM | 3404 | CA | LYS | B | 190 | 60.169 | 61.655 | 58.615 | 1.00 | 149.49 | C |
| ATOM | 3405 | CB | LYS | B | 190 | 58.888 | 62.014 | 59.399 | 1.00 | 148.27 | C |
| ATOM | 3406 | CG | LYS | B | 190 | 57.901 | 60.843 | 59.598 | 1.00 | 144.16 | C |
| ATOM | 3407 | CD | LYS | B | 190 | 58.579 | 59.482 | 59.320 | 1.00 | 140.21 | C |
| ATOM | 3408 | CE | LYS | B | 190 | 58.162 | 58.998 | 57.855 | 1.00 | 136.48 | C |
| ATOM | 3409 | NZ | LYS | B | 190 | 59.321 | 58.411 | 57.116 | 1.00 | 127.90 | N |
| ATOM | 3410 | C | LYS | B | 190 | 59.867 | 61.454 | 57.121 | 1.00 | 148.88 | C |
| ATOM | 3411 | O | LYS | B | 190 | 58.843 | 60.853 | 56.707 | 1.00 | 149.06 | O |
| ATOM | 3412 | N | GLU | B | 191 | 60.802 | 61.967 | 56.319 | 1.00 | 144.79 | N |
| ATOM | 3413 | CA | GLU | B | 191 | 60.777 | 61.760 | 54.865 | 1.00 | 139.18 | C |
| ATOM | 3414 | CB | GLU | B | 191 | 62.152 | 61.156 | 54.452 | 1.00 | 137.87 | C |
| ATOM | 3415 | CG | GLU | B | 191 | 62.316 | 59.655 | 55.104 | 1.00 | 129.73 | C |
| ATOM | 3416 | CD | GLU | B | 191 | 63.132 | 58.746 | 54.199 | 1.00 | 129.72 | C |
| ATOM | 3417 | OE1 | GLU | B | 191 | 63.547 | 59.232 | 53.094 | 1.00 | 125.01 | O |
| ATOM | 3418 | OE2 | GLU | B | 191 | 63.367 | 57.571 | 54.598 | 1.00 | 126.62 | O |
| ATOM | 3419 | C | GLU | B | 191 | 59.755 | 60.734 | 54.429 | 1.00 | 138.00 | C |
| ATOM | 3420 | O | GLU | B | 191 | 59.671 | 59.601 | 54.984 | 1.00 | 137.79 | O |
| ATOM | 3421 | N | GLU | B | 192 | 59.239 | 61.042 | 53.185 | 1.00 | 132.86 | N |
| ATOM | 3422 | CA | GLU | B | 192 | 58.479 | 60.031 | 52.484 | 1.00 | 123.04 | C |
| ATOM | 3423 | CB | GLU | B | 192 | 57.318 | 60.720 | 51.727 | 1.00 | 122.20 | C |
| ATOM | 3424 | CG | GLU | B | 192 | 56.012 | 59.941 | 51.832 | 1.00 | 119.74 | C |
| ATOM | 3425 | CD | GLU | B | 192 | 55.227 | 60.326 | 53.067 | 1.00 | 123.40 | C |
| ATOM | 3426 | OE1 | GLU | B | 192 | 55.252 | 59.564 | 54.066 | 1.00 | 121.76 | O |
| ATOM | 3427 | OE2 | GLU | B | 192 | 54.541 | 61.370 | 53.012 | 1.00 | 121.71 | O |
| ATOM | 3428 | C | GLU | B | 192 | 59.372 | 59.148 | 51.543 | 1.00 | 111.43 | C |
| ATOM | 3429 | O | GLU | B | 192 | 58.944 | 58.123 | 50.974 | 1.00 | 110.28 | O |
| ATOM | 3430 | N | GLU | B | 193 | 60.597 | 59.591 | 51.302 | 1.00 | 90.69 | N |
| ATOM | 3431 | CA | GLU | B | 193 | 61.134 | 59.271 | 50.000 | 1.00 | 85.92 | C |
| ATOM | 3432 | CB | GLU | B | 193 | 61.370 | 60.527 | 49.182 | 1.00 | 89.31 | C |
| ATOM | 3433 | CG | GLU | B | 193 | 60.387 | 60.651 | 48.015 | 1.00 | 101.43 | C |
| ATOM | 3434 | CD | GLU | B | 193 | 58.925 | 60.701 | 48.456 | 1.00 | 118.99 | C |
| ATOM | 3435 | OE1 | GLU | B | 193 | 58.504 | 61.743 | 49.013 | 1.00 | 120.83 | O |
| ATOM | 3436 | OE2 | GLU | B | 193 | 58.174 | 59.730 | 48.193 | 1.00 | 116.94 | O |
| ATOM | 3437 | C | GLU | B | 193 | 62.408 | 58.540 | 50.196 | 1.00 | 79.56 | C |
| ATOM | 3438 | O | GLU | B | 193 | 62.748 | 58.236 | 51.320 | 1.00 | 83.60 | O |
| ATOM | 3439 | N | ALA | B | 194 | 63.100 | 58.253 | 49.092 | 1.00 | 78.45 | N |
| ATOM | 3440 | CA | ALA | B | 194 | 64.415 | 57.640 | 49.166 | 1.00 | 86.31 | C |
| ATOM | 3441 | CB | ALA | B | 194 | 64.404 | 56.480 | 50.208 | 1.00 | 84.48 | C |
| ATOM | 3442 | C | ALA | B | 194 | 64.816 | 57.136 | 47.761 | 1.00 | 86.24 | C |
| ATOM | 3443 | O | ALA | B | 194 | 64.481 | 57.752 | 46.740 | 1.00 | 88.20 | O |
| ATOM | 3444 | N | VAL | B | 195 | 65.526 | 56.000 | 47.726 | 1.00 | 79.09 | N |
| ATOM | 3445 | CA | VAL | B | 195 | 66.026 | 55.455 | 46.477 | 1.00 | 65.29 | C |
| ATOM | 3446 | CB | VAL | B | 195 | 67.153 | 56.308 | 45.916 | 1.00 | 67.29 | C |
| ATOM | 3447 | CG1 | VAL | B | 195 | 68.408 | 56.132 | 46.795 | 1.00 | 63.31 | C |
| ATOM | 3448 | CG2 | VAL | B | 195 | 67.418 | 55.964 | 44.452 | 1.00 | 54.16 | C |
| ATOM | 3449 | C | VAL | B | 195 | 66.465 | 53.998 | 46.620 | 1.00 | 63.94 | C |
| ATOM | 3450 | O | VAL | B | 195 | 65.944 | 53.148 | 45.919 | 1.00 | 78.40 | O |
| ATOM | 3451 | N | THR | B | 196 | 67.362 | 53.646 | 47.532 | 1.00 | 59.91 | N |
| ATOM | 3452 | CA | THR | B | 196 | 67.688 | 52.221 | 47.663 | 1.00 | 63.83 | C |
| ATOM | 3453 | CB | THR | B | 196 | 66.536 | 51.437 | 48.378 | 1.00 | 60.44 | C |
| ATOM | 3454 | OG1 | THR | B | 196 | 66.057 | 52.194 | 49.502 | 1.00 | 78.70 | O |
| ATOM | 3455 | CG2 | THR | B | 196 | 67.080 | 50.152 | 49.051 | 1.00 | 55.91 | C |
| ATOM | 3456 | C | THR | B | 196 | 67.996 | 51.556 | 46.306 | 1.00 | 66.88 | C |
| ATOM | 3457 | O | THR | B | 196 | 67.103 | 51.370 | 45.471 | 1.00 | 61.97 | O |
| ATOM | 3458 | N | ILE | B | 197 | 69.230 | 51.077 | 46.147 | 1.00 | 70.72 | N |
| ATOM | 3459 | CA | ILE | B | 197 | 69.590 | 50.282 | 44.978 | 1.00 | 61.84 | C |
| ATOM | 3460 | CB | ILE | B | 197 | 70.407 | 51.142 | 44.007 | 1.00 | 57.31 | C |
| ATOM | 3461 | CG1 | ILE | B | 197 | 71.296 | 52.126 | 44.785 | 1.00 | 62.05 | C |
| ATOM | 3462 | CD1 | ILE | B | 197 | 71.375 | 53.542 | 44.194 | 1.00 | 63.78 | C |
| ATOM | 3463 | CG2 | ILE | B | 197 | 69.442 | 51.916 | 43.136 | 1.00 | 69.65 | C |
| ATOM | 3464 | C | ILE | B | 197 | 70.341 | 49.023 | 45.394 | 1.00 | 53.91 | C |
| ATOM | 3465 | O | ILE | B | 197 | 71.313 | 49.090 | 46.144 | 1.00 | 57.74 | O |
| ATOM | 3466 | N | GLU | B | 198 | 69.881 | 47.869 | 44.929 | 1.00 | 49.11 | N |
| ATOM | 3467 | CA | GLU | B | 198 | 70.691 | 46.665 | 45.003 | 1.00 | 50.92 | C |
| ATOM | 3468 | CB | GLU | B | 198 | 69.816 | 45.487 | 45.367 | 1.00 | 48.66 | C |
| ATOM | 3469 | CG | GLU | B | 198 | 69.386 | 45.557 | 46.825 | 1.00 | 71.11 | C |
| ATOM | 3470 | CD | GLU | B | 198 | 68.195 | 44.663 | 47.118 | 1.00 | 94.43 | C |
| ATOM | 3471 | OE1 | GLU | B | 198 | 67.140 | 45.201 | 47.522 | 1.00 | 94.26 | O |
| ATOM | 3472 | OE2 | GLU | B | 198 | 68.310 | 43.427 | 46.944 | 1.00 | 100.00 | O |
| ATOM | 3473 | C | GLU | B | 198 | 71.429 | 46.366 | 43.714 | 1.00 | 53.05 | C |
| ATOM | 3474 | O | GLU | B | 198 | 70.993 | 45.552 | 42.924 | 1.00 | 56.06 | O |
| ATOM | 3475 | N | MET | B | 199 | 72.571 | 47.013 | 43.527 | 1.00 | 54.50 | N |
| ATOM | 3476 | CA | MET | B | 199 | 73.358 | 46.874 | 42.314 | 1.00 | 51.85 | C |
| ATOM | 3477 | CB | MET | B | 199 | 73.988 | 48.224 | 41.943 | 1.00 | 43.68 | C |
| ATOM | 3478 | CG | MET | B | 199 | 75.160 | 48.151 | 40.973 | 1.00 | 46.98 | C |
| ATOM | 3479 | SD | MET | B | 199 | 76.694 | 48.594 | 41.764 | 1.00 | 95.49 | S |
| ATOM | 3480 | CE | MET | B | 199 | 77.837 | 47.383 | 41.013 | 1.00 | 82.00 | C |
| ATOM | 3481 | C | MET | B | 199 | 74.442 | 45.816 | 42.538 | 1.00 | 58.00 | C |
| ATOM | 3482 | O | MET | B | 199 | 75.342 | 45.999 | 43.361 | 1.00 | 62.02 | O |
| ATOM | 3483 | N | ASN | B | 200 | 74.367 | 44.722 | 41.779 | 1.00 | 64.65 | N |
| ATOM | 3484 | CA | ASN | B | 200 | 75.542 | 43.874 | 41.541 | 1.00 | 72.70 | C |
| ATOM | 3485 | CB | ASN | B | 200 | 75.115 | 42.446 | 41.214 | 1.00 | 75.96 | C |
| ATOM | 3486 | CG | ASN | B | 200 | 73.976 | 41.996 | 42.083 | 1.00 | 88.03 | C |
| ATOM | 3487 | OD1 | ASN | B | 200 | 73.686 | 42.645 | 43.104 | 1.00 | 87.01 | O |
| ATOM | 3488 | ND2 | ASN | B | 200 | 73.279 | 40.932 | 41.662 | 1.00 | 90.67 | N |
| ATOM | 3489 | C | ASN | B | 200 | 76.569 | 44.359 | 40.519 | 1.00 | 73.13 | C |
| ATOM | 3490 | O | ASN | B | 200 | 77.766 | 44.150 | 40.706 | 1.00 | 84.98 | O |
| ATOM | 3491 | N | GLU | B | 201 | 76.115 | 44.935 | 39.410 | 1.00 | 64.26 | N |
| ATOM | 3492 | CA | GLU | B | 201 | 77.037 | 45.480 | 38.429 | 1.00 | 53.87 | C |
| ATOM | 3493 | CB | GLU | B | 201 | 77.287 | 44.471 | 37.304 | 1.00 | 55.28 | C |
| ATOM | 3494 | CG | GLU | B | 201 | 76.509 | 43.175 | 37.464 | 1.00 | 69.75 | C |
| ATOM | 3495 | CD | GLU | B | 201 | 76.395 | 42.410 | 36.162 | 1.00 | 90.49 | C |
| ATOM | 3496 | OE1 | GLU | B | 201 | 77.257 | 42.597 | 35.274 | 1.00 | 89.73 | O |
| ATOM | 3497 | OE2 | GLU | B | 201 | 75.438 | 41.621 | 36.027 | 1.00 | 86.95 | O |
| ATOM | 3498 | C | GLU | B | 201 | 76.429 | 46.741 | 37.873 | 1.00 | 48.63 | C |
| ATOM | 3499 | O | GLU | B | 201 | 75.240 | 46.797 | 37.597 | 1.00 | 46.22 | O |
| ATOM | 3500 | N | PRO | B | 202 | 77.271 | 47.732 | 37.649 | 1.00 | 44.73 | N |
| ATOM | 3501 | CA | PRO | B | 202 | 76.879 | 48.951 | 36.931 | 1.00 | 52.36 | C |
| ATOM | 3502 | CB | PRO | B | 202 | 78.204 | 49.712 | 36.807 | 1.00 | 52.99 | C |
| ATOM | 3503 | CG | PRO | B | 202 | 79.028 | 49.187 | 37.947 | 1.00 | 51.69 | C |
| ATOM | 3504 | CD | PRO | B | 202 | 78.683 | 47.727 | 38.050 | 1.00 | 40.14 | C |
| ATOM | 3505 | C | PRO | B | 202 | 76.266 | 48.709 | 35.539 | 1.00 | 54.74 | C |
| ATOM | 3506 | O | PRO | B | 202 | 76.843 | 47.945 | 34.746 | 1.00 | 62.71 | O |
| ATOM | 3507 | N | VAL | B | 203 | 75.125 | 49.347 | 35.255 | 1.00 | 50.23 | N |
| ATOM | 3508 | CA | VAL | B | 203 | 74.457 | 49.232 | 33.958 | 1.00 | 45.35 | C |
| ATOM | 3509 | CB | VAL | B | 203 | 73.097 | 48.554 | 34.060 | 1.00 | 36.13 | C |
| ATOM | 3510 | CG1 | VAL | B | 203 | 72.796 | 47.852 | 32.770 | 1.00 | 56.83 | C |
| ATOM | 3511 | CG2 | VAL | B | 203 | 73.062 | 47.576 | 35.200 | 1.00 | 48.43 | C |
| ATOM | 3512 | C | VAL | B | 203 | 74.247 | 50.584 | 33.302 | 1.00 | 45.83 | C |
| ATOM | 3513 | O | VAL | B | 203 | 74.167 | 51.599 | 33.982 | 1.00 | 57.42 | O |
| ATOM | 3514 | N | GLN | B | 204 | 74.169 | 50.588 | 31.975 | 1.00 | 39.08 | N |
| ATOM | 3515 | CA | GLN | B | 204 | 73.910 | 51.802 | 31.220 | 1.00 | 34.97 | C |
| ATOM | 3516 | CB | GLN | B | 204 | 75.218 | 52.537 | 30.893 | 1.00 | 33.97 | C |
| ATOM | 3517 | CG | GLN | B | 204 | 75.104 | 54.044 | 30.614 | 1.00 | 38.62 | C |
| ATOM | 3518 | CD | GLN | B | 204 | 76.390 | 54.663 | 30.029 | 1.00 | 50.96 | C |
| ATOM | 3519 | OE1 | GLN | B | 204 | 77.477 | 54.588 | 30.615 | 1.00 | 58.30 | O |
| ATOM | 3520 | NE2 | GLN | B | 204 | 76.244 | 55.315 | 28.890 | 1.00 | 53.65 | N |
| ATOM | 3521 | C | GLN | B | 204 | 73.195 | 51.388 | 29.945 | 1.00 | 38.02 | C |
| ATOM | 3522 | O | GLN | B | 204 | 73.797 | 50.819 | 29.035 | 1.00 | 45.71 | O |
| ATOM | 3523 | N | LEU | B | 205 | 71.908 | 51.708 | 29.873 | 1.00 | 33.45 | N |
| ATOM | 3524 | CA | LEU | B | 205 | 71.136 | 51.513 | 28.653 | 1.00 | 40.37 | C |
| ATOM | 3525 | CB | LEU | B | 205 | 70.181 | 50.348 | 28.864 | 1.00 | 46.96 | C |
| ATOM | 3526 | CG | LEU | B | 205 | 70.626 | 49.378 | 29.959 | 1.00 | 49.36 | C |
| ATOM | 3527 | CD1 | LEU | B | 205 | 69.989 | 49.787 | 31.277 | 1.00 | 69.94 | C |
| ATOM | 3528 | CD2 | LEU | B | 205 | 70.221 | 47.963 | 29.586 | 1.00 | 30.53 | C |
| ATOM | 3529 | C | LEU | B | 205 | 70.355 | 52.752 | 28.201 | 1.00 | 42.33 | C |
| ATOM | 3530 | O | LEU | B | 205 | 70.317 | 53.764 | 28.906 | 1.00 | 46.50 | O |
| ATOM | 3531 | N | THR | B | 206 | 69.738 | 52.682 | 27.019 | 1.00 | 38.39 | N |
| ATOM | 3532 | CA | THR | B | 206 | 68.888 | 53.775 | 26.554 | 1.00 | 34.45 | C |
| ATOM | 3533 | CB | THR | B | 206 | 69.448 | 54.362 | 25.254 | 1.00 | 33.48 | C |
| ATOM | 3534 | OG1 | THR | B | 206 | 70.856 | 54.564 | 25.423 | 1.00 | 37.40 | O |
| ATOM | 3535 | CG2 | THR | B | 206 | 68.897 | 55.780 | 25.007 | 1.00 | 27.80 | C |
| ATOM | 3536 | C | THR | B | 206 | 67.465 | 53.295 | 26.366 | 1.00 | 28.09 | C |
| ATOM | 3537 | O | THR | B | 206 | 67.257 | 52.173 | 25.912 | 1.00 | 37.06 | O |
| ATOM | 3538 | N | PHE | B | 207 | 66.488 | 54.106 | 26.763 | 1.00 | 25.66 | N |
| ATOM | 3539 | CA | PHE | B | 207 | 65.091 | 53.736 | 26.586 | 1.00 | 24.03 | C |
| ATOM | 3540 | CB | PHE | B | 207 | 64.484 | 53.349 | 27.920 | 1.00 | 22.36 | C |
| ATOM | 3541 | CG | PHE | B | 207 | 65.165 | 52.197 | 28.577 | 1.00 | 28.06 | C |
| ATOM | 3542 | CD1 | PHE | B | 207 | 64.851 | 50.880 | 28.223 | 1.00 | 27.08 | C |
| ATOM | 3543 | CE1 | PHE | B | 207 | 65.456 | 49.793 | 28.854 | 1.00 | 37.53 | C |
| ATOM | 3544 | CZ | PHE | B | 207 | 66.423 | 50.021 | 29.821 | 1.00 | 34.26 | C |
| ATOM | 3545 | CE2 | PHE | B | 207 | 66.685 | 51.345 | 30.229 | 1.00 | 55.42 | C |
| ATOM | 3546 | CD2 | PHE | B | 207 | 66.059 | 52.423 | 29.601 | 1.00 | 31.57 | C |
| ATOM | 3547 | C | PHE | B | 207 | 64.279 | 54.859 | 25.977 | 1.00 | 23.00 | C |
| ATOM | 3548 | O | PHE | B | 207 | 64.668 | 56.036 | 26.082 | 1.00 | 18.91 | O |
| ATOM | 3549 | N | ALA | B | 208 | 63.147 | 54.495 | 25.364 | 1.00 | 18.91 | N |
| ATOM | 3550 | CA | ALA | B | 208 | 62.198 | 55.491 | 24.835 | 1.00 | 21.49 | C |
| ATOM | 3551 | CB | ALA | B | 208 | 61.417 | 54.894 | 23.746 | 1.00 | 20.41 | C |
| ATOM | 3552 | C | ALA | B | 208 | 61.247 | 56.143 | 25.847 | 1.00 | 25.89 | C |
| ATOM | 3553 | O | ALA | B | 208 | 60.565 | 55.448 | 26.598 | 1.00 | 35.69 | O |
| ATOM | 3554 | N | LEU | B | 209 | 61.174 | 57.470 | 25.859 | 1.00 | 27.44 | N |
| ATOM | 3555 | CA | LEU | B | 209 | 60.491 | 58.175 | 26.948 | 1.00 | 30.95 | C |
| ATOM | 3556 | CB | LEU | B | 209 | 60.989 | 59.630 | 27.088 | 1.00 | 31.79 | C |
| ATOM | 3557 | CG | LEU | B | 209 | 62.247 | 59.911 | 27.921 | 1.00 | 32.77 | C |
| ATOM | 3558 | CD1 | LEU | B | 209 | 62.821 | 61.275 | 27.572 | 1.00 | 28.55 | C |
| ATOM | 3559 | CD2 | LEU | B | 209 | 61.957 | 59.822 | 29.397 | 1.00 | 42.59 | C |
| ATOM | 3560 | C | LEU | B | 209 | 58.987 | 58.142 | 26.678 | 1.00 | 32.04 | C |
| ATOM | 3561 | O | LEU | B | 209 | 58.167 | 58.250 | 27.596 | 1.00 | 32.99 | O |
| ATOM | 3562 | N | ARG | B | 210 | 58.640 | 58.045 | 25.398 | 1.00 | 30.95 | N |
| ATOM | 3563 | CA | ARG | B | 210 | 57.251 | 57.935 | 25.014 | 1.00 | 25.48 | C |
| ATOM | 3564 | CB | ARG | B | 210 | 57.142 | 57.765 | 23.493 | 1.00 | 21.92 | C |
| ATOM | 3565 | CG | ARG | B | 210 | 55.723 | 57.803 | 22.937 | 1.00 | 35.08 | C |
| ATOM | 3566 | CD | ARG | B | 210 | 55.665 | 57.894 | 21.414 | 1.00 | 67.55 | C |
| ATOM | 3567 | NE | ARG | B | 210 | 54.738 | 56.929 | 20.817 | 1.00 | 66.43 | N |
| ATOM | 3568 | CZ | ARG | B | 210 | 53.516 | 57.232 | 20.380 | 1.00 | 82.86 | C |
| ATOM | 3569 | NH1 | ARG | B | 210 | 53.034 | 58.470 | 20.536 | 1.00 | 91.21 | N |
| ATOM | 3570 | NH2 | ARG | B | 210 | 52.759 | 56.293 | 19.805 | 1.00 | 87.00 | N |
| ATOM | 3571 | C | ARG | B | 210 | 56.637 | 56.759 | 25.779 | 1.00 | 27.77 | C |
| ATOM | 3572 | O | ARG | B | 210 | 55.553 | 56.857 | 26.332 | 1.00 | 27.77 | O |
| ATOM | 3573 | N | TYR | B | 211 | 57.317 | 55.625 | 25.810 | 1.00 | 30.21 | N |
| ATOM | 3574 | CA | TYR | B | 211 | 56.671 | 54.421 | 26.295 | 1.00 | 28.05 | C |
| ATOM | 3575 | CB | TYR | B | 211 | 57.380 | 53.173 | 25.768 | 1.00 | 28.85 | C |
| ATOM | 3576 | CG | TYR | B | 211 | 56.945 | 52.841 | 24.371 | 1.00 | 31.14 | C |
| ATOM | 3577 | CD1 | TYR | B | 211 | 55.771 | 52.143 | 24.131 | 1.00 | 41.46 | C |
| ATOM | 3578 | CE1 | TYR | B | 211 | 55.370 | 51.836 | 22.846 | 1.00 | 33.29 | C |
| ATOM | 3579 | CZ | TYR | B | 211 | 56.114 | 52.286 | 21.787 | 1.00 | 29.26 | C |
| ATOM | 3580 | OH | TYR | B | 211 | 55.701 | 52.025 | 20.506 | 1.00 | 73.78 | O |
| ATOM | 3581 | CE2 | TYR | B | 211 | 57.268 | 53.011 | 22.003 | 1.00 | 40.20 | C |
| ATOM | 3582 | CD2 | TYR | B | 211 | 57.656 | 53.309 | 23.292 | 1.00 | 14.59 | C |
| ATOM | 3583 | C | TYR | B | 211 | 56.725 | 54.480 | 27.800 | 1.00 | 28.26 | C |
| ATOM | 3584 | O | TYR | B | 211 | 55.814 | 54.002 | 28.484 | 1.00 | 29.33 | O |
| ATOM | 3585 | N | LEU | B | 212 | 57.803 | 55.061 | 28.317 | 1.00 | 29.53 | N |
| ATOM | 3586 | CA | LEU | B | 212 | 57.923 | 55.248 | 29.765 | 1.00 | 22.16 | C |
| ATOM | 3587 | CB | LEU | B | 212 | 59.277 | 55.860 | 30.139 | 1.00 | 11.57 | C |
| ATOM | 3588 | CG | LEU | B | 212 | 60.399 | 54.832 | 30.316 | 1.00 | 21.46 | C |
| ATOM | 3589 | CD1 | LEU | B | 212 | 61.720 | 55.541 | 30.532 | 1.00 | 19.86 | C |
| ATOM | 3590 | CD2 | LEU | B | 212 | 60.092 | 53.909 | 31.469 | 1.00 | 21.24 | C |
| ATOM | 3591 | C | LEU | B | 212 | 56.779 | 56.120 | 30.273 | 1.00 | 22.95 | C |
| ATOM | 3592 | O | LEU | B | 212 | 56.316 | 55.951 | 31.400 | 1.00 | 24.48 | O |
| ATOM | 3593 | N | ASN | B | 213 | 56.331 | 57.058 | 29.441 | 1.00 | 18.27 | N |
| ATOM | 3594 | CA | ASN | B | 213 | 55.280 | 57.953 | 29.861 | 1.00 | 21.00 | C |
| ATOM | 3595 | CB | ASN | B | 213 | 55.163 | 59.128 | 28.903 | 1.00 | 27.21 | C |
| ATOM | 3596 | CG | ASN | B | 213 | 56.164 | 60.227 | 29.208 | 1.00 | 31.36 | C |
| ATOM | 3597 | OD1 | ASN | B | 213 | 56.866 | 60.174 | 30.208 | 1.00 | 32.77 | O |
| ATOM | 3598 | ND2 | ASN | B | 213 | 56.199 | 61.252 | 28.361 | 1.00 | 52.96 | N |
| ATOM | 3599 | C | ASN | B | 213 | 53.977 | 57.188 | 29.944 | 1.00 | 28.66 | C |
| ATOM | 3600 | O | ASN | B | 213 | 53.014 | 57.639 | 30.573 | 1.00 | 31.52 | O |
| ATOM | 3601 | N | PHE | B | 214 | 53.942 | 56.048 | 29.260 | 1.00 | 34.66 | N |
| ATOM | 3602 | CA | PHE | B | 214 | 52.734 | 55.237 | 29.211 | 1.00 | 34.05 | C |
| ATOM | 3603 | CB | PHE | B | 214 | 52.780 | 54.281 | 28.013 | 1.00 | 38.05 | C |
| ATOM | 3604 | CG | PHE | B | 214 | 52.421 | 54.931 | 26.710 | 1.00 | 39.52 | C |
| ATOM | 3605 | CD1 | PHE | B | 214 | 51.540 | 56.004 | 26.680 | 1.00 | 39.78 | C |
| ATOM | 3606 | CE1 | PHE | B | 214 | 51.202 | 56.607 | 25.479 | 1.00 | 56.15 | C |
| ATOM | 3607 | CZ | PHE | B | 214 | 51.775 | 56.162 | 24.295 | 1.00 | 51.18 | C |
| ATOM | 3608 | CE2 | PHE | B | 214 | 52.692 | 55.118 | 24.319 | 1.00 | 53.87 | C |
| ATOM | 3609 | CD2 | PHE | B | 214 | 53.009 | 54.508 | 25.522 | 1.00 | 56.97 | C |
| ATOM | 3610 | C | PHE | B | 214 | 52.719 | 54.445 | 30.492 | 1.00 | 30.11 | C |
| ATOM | 3611 | O | PHE | B | 214 | 51.672 | 53.993 | 30.962 | 1.00 | 26.92 | O |
| ATOM | 3612 | N | PHE | B | 215 | 53.913 | 54.264 | 31.037 | 1.00 | 35.33 | N |
| ATOM | 3613 | CA | PHE | B | 215 | 54.073 | 53.376 | 32.172 | 1.00 | 37.73 | C |
| ATOM | 3614 | CB | PHE | B | 215 | 55.517 | 52.895 | 32.230 | 1.00 | 38.53 | C |
| ATOM | 3615 | CG | PHE | B | 215 | 55.907 | 51.983 | 31.089 | 1.00 | 32.18 | C |
| ATOM | 3616 | CD1 | PHE | B | 215 | 54.953 | 51.526 | 30.189 | 1.00 | 21.29 | C |
| ATOM | 3617 | CE1 | PHE | B | 215 | 55.300 | 50.656 | 29.172 | 1.00 | 38.95 | C |
| ATOM | 3618 | CZ | PHE | B | 215 | 56.624 | 50.209 | 29.057 | 1.00 | 42.08 | C |
| ATOM | 3619 | CE2 | PHE | B | 215 | 57.593 | 50.648 | 29.965 | 1.00 | 14.34 | C |
| ATOM | 3620 | CD2 | PHE | B | 215 | 57.231 | 51.548 | 30.953 | 1.00 | 11.22 | C |
| ATOM | 3621 | C | PHE | B | 215 | 53.696 | 54.101 | 33.456 | 1.00 | 34.66 | C |
| ATOM | 3622 | O | PHE | B | 215 | 53.153 | 53.500 | 34.383 | 1.00 | 33.65 | O |
| ATOM | 3623 | N | THR | B | 216 | 54.011 | 55.393 | 33.502 | 1.00 | 28.03 | N |
| ATOM | 3624 | CA | THR | B | 216 | 53.714 | 56.217 | 34.664 | 1.00 | 25.36 | C |
| ATOM | 3625 | CB | THR | B | 216 | 54.462 | 57.558 | 34.566 | 1.00 | 11.56 | C |
| ATOM | 3626 | OG1 | THR | B | 216 | 54.190 | 58.175 | 33.306 | 1.00 | 24.41 | O |
| ATOM | 3627 | CG2 | THR | B | 216 | 55.913 | 57.302 | 34.477 | 1.00 | 17.54 | C |
| ATOM | 3628 | C | THR | B | 216 | 52.212 | 56.463 | 34.849 | 1.00 | 30.22 | C |
| ATOM | 3629 | O | THR | B | 216 | 51.812 | 57.281 | 35.686 | 1.00 | 35.97 | O |
| ATOM | 3630 | N | LYS | B | 217 | 51.383 | 55.822 | 34.031 | 1.00 | 20.28 | N |
| ATOM | 3631 | CA | LYS | B | 217 | 49.939 | 55.974 | 34.191 | 1.00 | 27.38 | C |
| ATOM | 3632 | CB | LYS | B | 217 | 49.221 | 55.731 | 32.865 | 1.00 | 22.58 | C |
| ATOM | 3633 | CG | LYS | B | 217 | 49.378 | 56.815 | 31.852 | 1.00 | 19.83 | C |
| ATOM | 3634 | CD | LYS | B | 217 | 48.340 | 57.894 | 32.100 | 1.00 | 48.66 | C |
| ATOM | 3635 | CE | LYS | B | 217 | 48.774 | 59.241 | 31.559 | 1.00 | 48.16 | C |
| ATOM | 3636 | NZ | LYS | B | 217 | 48.423 | 59.259 | 30.121 | 1.00 | 77.29 | N |
| ATOM | 3637 | C | LYS | B | 217 | 49.429 | 54.987 | 35.265 | 1.00 | 35.80 | C |
| ATOM | 3638 | O | LYS | B | 217 | 48.226 | 54.933 | 35.588 | 1.00 | 34.51 | O |
| ATOM | 3639 | N | ALA | B | 218 | 50.367 | 54.246 | 35.854 | 1.00 | 30.51 | N |
| ATOM | 3640 | CA | ALA | B | 218 | 50.036 | 53.317 | 36.911 | 1.00 | 22.67 | C |
| ATOM | 3641 | CB | ALA | B | 218 | 50.736 | 51.995 | 36.638 | 1.00 | 25.14 | C |
| ATOM | 3642 | C | ALA | B | 218 | 50.469 | 53.904 | 38.250 | 1.00 | 21.88 | C |
| ATOM | 3643 | O | ALA | B | 218 | 50.590 | 53.207 | 39.255 | 1.00 | 29.23 | O |
| ATOM | 3644 | N | THR | B | 219 | 50.710 | 55.206 | 38.268 | 1.00 | 18.23 | N |
| ATOM | 3645 | CA | THR | B | 219 | 51.163 | 55.863 | 39.504 | 1.00 | 27.05 | C |
| ATOM | 3646 | CB | THR | B | 219 | 51.499 | 57.339 | 39.258 | 1.00 | 22.17 | C |
| ATOM | 3647 | OG1 | THR | B | 219 | 52.763 | 57.436 | 38.586 | 1.00 | 34.04 | O |
| ATOM | 3648 | CG2 | THR | B | 219 | 51.734 | 58.036 | 40.545 | 1.00 | 22.04 | C |
| ATOM | 3649 | C | THR | B | 219 | 50.176 | 55.752 | 40.658 | 1.00 | 23.43 | C |
| ATOM | 3650 | O | THR | B | 219 | 50.528 | 55.330 | 41.737 | 1.00 | 29.67 | O |
| ATOM | 3651 | N | PRO | B | 220 | 48.928 | 56.117 | 40.430 | 1.00 | 27.46 | N |
| ATOM | 3652 | CA | PRO | B | 220 | 47.908 | 56.011 | 41.464 | 1.00 | 33.17 | C |
| ATOM | 3653 | CB | PRO | B | 220 | 46.624 | 56.304 | 40.686 | 1.00 | 34.59 | C |
| ATOM | 3654 | CG | PRO | B | 220 | 47.081 | 57.172 | 39.586 | 1.00 | 14.95 | C |
| ATOM | 3655 | CD | PRO | B | 220 | 48.365 | 56.602 | 39.163 | 1.00 | 31.98 | C |
| ATOM | 3656 | C | PRO | B | 220 | 47.865 | 54.624 | 42.106 | 1.00 | 33.75 | C |
| ATOM | 3657 | O | PRO | B | 220 | 47.220 | 54.474 | 43.156 | 1.00 | 38.00 | O |
| ATOM | 3658 | N | LEU | B | 221 | 48.504 | 53.634 | 41.480 | 1.00 | 24.39 | N |
| ATOM | 3659 | CA | LEU | B | 221 | 48.276 | 52.248 | 41.886 | 1.00 | 24.93 | C |
| ATOM | 3660 | CB | LEU | B | 221 | 48.514 | 51.263 | 40.738 | 1.00 | 11.82 | C |
| ATOM | 3661 | CG | LEU | B | 221 | 47.228 | 50.739 | 40.073 | 1.00 | 32.82 | C |
| ATOM | 3662 | CD1 | LEU | B | 221 | 47.430 | 49.962 | 38.805 | 1.00 | 27.68 | C |
| ATOM | 3663 | CD2 | LEU | B | 221 | 46.422 | 49.880 | 41.004 | 1.00 | 62.17 | C |
| ATOM | 3664 | C | LEU | B | 221 | 49.117 | 51.902 | 43.106 | 1.00 | 28.20 | C |
| ATOM | 3665 | O | LEU | B | 221 | 48.737 | 51.074 | 43.917 | 1.00 | 33.69 | O |
| ATOM | 3666 | N | SER | B | 222 | 50.236 | 52.611 | 43.269 | 1.00 | 34.16 | N |
| ATOM | 3667 | CA | SER | B | 222 | 51.267 | 52.187 | 44.210 | 1.00 | 38.01 | C |
| ATOM | 3668 | CB | SER | B | 222 | 52.125 | 51.080 | 43.573 | 1.00 | 44.61 | C |
| ATOM | 3669 | OG | SER | B | 222 | 52.771 | 50.269 | 44.544 | 1.00 | 50.16 | O |
| ATOM | 3670 | C | SER | B | 222 | 52.140 | 53.398 | 44.579 | 1.00 | 42.94 | C |
| ATOM | 3671 | O | SER | B | 222 | 52.284 | 54.339 | 43.804 | 1.00 | 46.38 | O |
| ATOM | 3672 | N | SER | B | 223 | 52.666 | 53.438 | 45.795 | 1.00 | 45.00 | N |
| ATOM | 3673 | CA | SER | B | 223 | 53.309 | 54.675 | 46.238 | 1.00 | 45.29 | C |
| ATOM | 3674 | CB | SER | B | 223 | 53.252 | 54.790 | 47.762 | 1.00 | 46.61 | C |
| ATOM | 3675 | OG | SER | B | 223 | 51.937 | 55.186 | 48.169 | 1.00 | 66.90 | O |
| ATOM | 3676 | C | SER | B | 223 | 54.758 | 54.723 | 45.750 | 1.00 | 47.10 | C |
| ATOM | 3677 | O | SER | B | 223 | 55.373 | 55.790 | 45.622 | 1.00 | 45.71 | O |
| ATOM | 3678 | N | THR | B | 224 | 55.306 | 53.540 | 45.495 | 1.00 | 53.42 | N |
| ATOM | 3679 | CA | THR | B | 224 | 56.658 | 53.412 | 44.957 | 1.00 | 53.61 | C |
| ATOM | 3680 | CB | THR | B | 224 | 57.670 | 53.007 | 46.065 | 1.00 | 57.87 | C |
| ATOM | 3681 | OG1 | THR | B | 224 | 57.120 | 51.947 | 46.870 | 1.00 | 78.44 | O |
| ATOM | 3682 | CG2 | THR | B | 224 | 57.868 | 54.148 | 47.066 | 1.00 | 52.17 | C |
| ATOM | 3683 | C | THR | B | 224 | 56.710 | 52.418 | 43.786 | 1.00 | 46.84 | C |
| ATOM | 3684 | O | THR | B | 224 | 55.804 | 51.619 | 43.555 | 1.00 | 28.91 | O |
| ATOM | 3685 | N | VAL | B | 225 | 57.759 | 52.535 | 42.989 | 1.00 | 45.35 | N |
| ATOM | 3686 | CA | VAL | B | 225 | 57.856 | 51.761 | 41.764 | 1.00 | 35.70 | C |
| ATOM | 3687 | CB | VAL | B | 225 | 57.691 | 52.649 | 40.514 | 1.00 | 38.41 | C |
| ATOM | 3688 | CG1 | VAL | B | 225 | 58.519 | 53.942 | 40.627 | 1.00 | 32.48 | C |
| ATOM | 3689 | CG2 | VAL | B | 225 | 58.129 | 51.904 | 39.294 | 1.00 | 19.62 | C |
| ATOM | 3690 | C | VAL | B | 225 | 59.261 | 51.234 | 41.746 | 1.00 | 42.75 | C |
| ATOM | 3691 | O | VAL | B | 225 | 60.198 | 51.922 | 42.163 | 1.00 | 46.28 | O |
| ATOM | 3692 | N | THR | B | 226 | 59.410 | 49.999 | 41.300 | 1.00 | 44.09 | N |
| ATOM | 3693 | CA | THR | B | 226 | 60.742 | 49.434 | 41.214 | 1.00 | 44.79 | C |
| ATOM | 3694 | CB | THR | B | 226 | 60.766 | 48.085 | 41.942 | 1.00 | 42.20 | C |
| ATOM | 3695 | OG1 | THR | B | 226 | 59.520 | 47.421 | 41.706 | 1.00 | 63.22 | O |
| ATOM | 3696 | CG2 | THR | B | 226 | 60.703 | 48.303 | 43.449 | 1.00 | 42.82 | C |
| ATOM | 3697 | C | THR | B | 226 | 61.051 | 49.296 | 39.734 | 1.00 | 40.84 | C |
| ATOM | 3698 | O | THR | B | 226 | 60.162 | 48.927 | 38.954 | 1.00 | 50.13 | O |
| ATOM | 3699 | N | LEU | B | 227 | 62.255 | 49.702 | 39.336 | 1.00 | 30.60 | N |
| ATOM | 3700 | CA | LEU | B | 227 | 62.761 | 49.406 | 37.993 | 1.00 | 34.67 | C |
| ATOM | 3701 | CB | LEU | B | 227 | 63.079 | 50.707 | 37.263 | 1.00 | 41.62 | C |
| ATOM | 3702 | CG | LEU | B | 227 | 63.000 | 51.952 | 38.132 | 1.00 | 36.01 | C |
| ATOM | 3703 | CD1 | LEU | B | 227 | 64.060 | 52.910 | 37.637 | 1.00 | 37.49 | C |
| ATOM | 3704 | CD2 | LEU | B | 227 | 61.631 | 52.542 | 37.935 | 1.00 | 57.55 | C |
| ATOM | 3705 | C | LEU | B | 227 | 63.988 | 48.496 | 37.953 | 1.00 | 28.46 | C |
| ATOM | 3706 | O | LEU | B | 227 | 64.976 | 48.759 | 38.609 | 1.00 | 32.37 | O |
| ATOM | 3707 | N | SER | B | 228 | 63.961 | 47.457 | 37.135 | 1.00 | 35.50 | N |
| ATOM | 3708 | CA | SER | B | 228 | 65.092 | 46.537 | 37.106 | 1.00 | 44.18 | C |
| ATOM | 3709 | CB | SER | B | 228 | 64.708 | 45.159 | 37.651 | 1.00 | 36.51 | C |
| ATOM | 3710 | OG | SER | B | 228 | 63.694 | 45.312 | 38.635 | 1.00 | 55.84 | O |
| ATOM | 3711 | C | SER | B | 228 | 65.645 | 46.404 | 35.701 | 1.00 | 50.82 | C |
| ATOM | 3712 | O | SER | B | 228 | 64.904 | 46.159 | 34.751 | 1.00 | 54.92 | O |
| ATOM | 3713 | N | MET | B | 229 | 66.960 | 46.565 | 35.595 | 1.00 | 55.59 | N |
| ATOM | 3714 | CA | MET | B | 229 | 67.680 | 46.477 | 34.328 | 1.00 | 52.35 | C |
| ATOM | 3715 | CB | MET | B | 229 | 67.934 | 47.895 | 33.790 | 1.00 | 53.23 | C |
| ATOM | 3716 | CG | MET | B | 229 | 67.717 | 49.020 | 34.808 | 1.00 | 47.65 | C |
| ATOM | 3717 | SD | MET | B | 229 | 68.859 | 50.389 | 34.509 | 1.00 | 61.80 | S |
| ATOM | 3718 | CE | MET | B | 229 | 69.049 | 50.985 | 36.214 | 1.00 | 42.74 | C |
| ATOM | 3719 | C | MET | B | 229 | 69.016 | 45.720 | 34.471 | 1.00 | 54.35 | C |
| ATOM | 3720 | O | MET | B | 229 | 69.572 | 45.578 | 35.561 | 1.00 | 53.44 | O |
| ATOM | 3721 | N | SER | B | 230 | 69.551 | 45.249 | 33.354 | 1.00 | 51.26 | N |
| ATOM | 3722 | CA | SER | B | 230 | 70.493 | 44.157 | 33.440 | 1.00 | 46.70 | C |
| ATOM | 3723 | CB | SER | B | 230 | 69.734 | 42.848 | 33.757 | 1.00 | 31.78 | C |
| ATOM | 3724 | OG | SER | B | 230 | 70.595 | 41.712 | 33.804 | 1.00 | 55.22 | O |
| ATOM | 3725 | C | SER | B | 230 | 71.220 | 44.040 | 32.114 | 1.00 | 47.92 | C |
| ATOM | 3726 | O | SER | B | 230 | 71.769 | 42.991 | 31.804 | 1.00 | 59.49 | O |
| ATOM | 3727 | N | ALA | B | 231 | 71.233 | 45.092 | 31.305 | 1.00 | 46.93 | N |
| ATOM | 3728 | CA | ALA | B | 231 | 72.089 | 45.068 | 30.108 | 1.00 | 45.65 | C |
| ATOM | 3729 | CB | ALA | B | 231 | 73.579 | 44.987 | 30.485 | 1.00 | 35.71 | C |
| ATOM | 3730 | C | ALA | B | 231 | 71.730 | 43.944 | 29.137 | 1.00 | 40.59 | C |
| ATOM | 3731 | O | ALA | B | 231 | 71.743 | 42.761 | 29.473 | 1.00 | 41.74 | O |
| ATOM | 3732 | N | ASP | B | 232 | 71.390 | 44.327 | 27.918 | 1.00 | 41.19 | N |
| ATOM | 3733 | CA | ASP | B | 232 | 71.112 | 43.333 | 26.895 | 1.00 | 53.89 | C |
| ATOM | 3734 | CB | ASP | B | 232 | 72.199 | 42.249 | 26.903 | 1.00 | 67.98 | C |
| ATOM | 3735 | CG | ASP | B | 232 | 73.368 | 42.568 | 25.959 | 1.00 | 75.36 | C |
| ATOM | 3736 | OD1 | ASP | B | 232 | 73.177 | 42.467 | 24.728 | 1.00 | 66.06 | O |
| ATOM | 3737 | OD2 | ASP | B | 232 | 74.529 | 42.839 | 26.361 | 1.00 | 69.77 | O |
| ATOM | 3738 | C | ASP | B | 232 | 69.756 | 42.665 | 27.095 | 1.00 | 49.63 | C |
| ATOM | 3739 | O | ASP | B | 232 | 69.315 | 41.878 | 26.246 | 1.00 | 42.19 | O |
| ATOM | 3740 | N | VAL | B | 233 | 69.095 | 42.966 | 28.209 | 1.00 | 48.23 | N |
| ATOM | 3741 | CA | VAL | B | 233 | 67.702 | 42.546 | 28.380 | 1.00 | 47.01 | C |
| ATOM | 3742 | CB | VAL | B | 233 | 67.532 | 41.594 | 29.582 | 1.00 | 46.69 | C |
| ATOM | 3743 | CG1 | VAL | B | 233 | 68.719 | 40.599 | 29.645 | 1.00 | 48.22 | C |
| ATOM | 3744 | CG2 | VAL | B | 233 | 67.410 | 42.411 | 30.897 | 1.00 | 56.83 | C |
| ATOM | 3745 | C | VAL | B | 233 | 66.794 | 43.755 | 28.553 | 1.00 | 42.19 | C |
| ATOM | 3746 | O | VAL | B | 233 | 67.256 | 44.857 | 28.842 | 1.00 | 46.92 | O |
| ATOM | 3747 | N | PRO | B | 234 | 65.497 | 43.540 | 28.375 | 1.00 | 38.39 | N |
| ATOM | 3748 | CA | PRO | B | 234 | 64.498 | 44.595 | 28.584 | 1.00 | 29.89 | C |
| ATOM | 3749 | CB | PRO | B | 234 | 63.206 | 43.965 | 28.028 | 1.00 | 13.65 | C |
| ATOM | 3750 | CG | PRO | B | 234 | 63.416 | 42.509 | 28.145 | 1.00 | 11.56 | C |
| ATOM | 3751 | CD | PRO | B | 234 | 64.892 | 42.264 | 27.951 | 1.00 | 42.94 | C |
| ATOM | 3752 | C | PRO | B | 234 | 64.370 | 45.067 | 30.052 | 1.00 | 25.15 | C |
| ATOM | 3753 | O | PRO | B | 234 | 64.620 | 44.347 | 31.015 | 1.00 | 27.15 | O |
| ATOM | 3754 | N | LEU | B | 235 | 64.054 | 46.337 | 30.211 | 1.00 | 24.00 | N |
| ATOM | 3755 | CA | LEU | B | 235 | 63.874 | 46.910 | 31.522 | 1.00 | 24.80 | C |
| ATOM | 3756 | CB | LEU | B | 235 | 63.988 | 48.432 | 31.419 | 1.00 | 25.41 | C |
| ATOM | 3757 | CG | LEU | B | 235 | 63.233 | 49.267 | 32.439 | 1.00 | 11.09 | C |
| ATOM | 3758 | CD1 | LEU | B | 235 | 63.928 | 49.178 | 33.770 | 1.00 | 26.13 | C |
| ATOM | 3759 | CD2 | LEU | B | 235 | 63.233 | 50.692 | 31.980 | 1.00 | 19.84 | C |
| ATOM | 3760 | C | LEU | B | 235 | 62.498 | 46.547 | 32.040 | 1.00 | 32.54 | C |
| ATOM | 3761 | O | LEU | B | 235 | 61.549 | 46.505 | 31.251 | 1.00 | 33.87 | O |
| ATOM | 3762 | N | VAL | B | 236 | 62.390 | 46.357 | 33.361 | 1.00 | 33.39 | N |
| ATOM | 3763 | CA | VAL | B | 236 | 61.124 | 46.002 | 33.979 | 1.00 | 35.05 | C |
| ATOM | 3764 | CB | VAL | B | 236 | 61.224 | 44.653 | 34.699 | 1.00 | 39.06 | C |
| ATOM | 3765 | CG1 | VAL | B | 236 | 59.844 | 44.017 | 34.872 | 1.00 | 38.57 | C |
| ATOM | 3766 | CG2 | VAL | B | 236 | 62.153 | 43.721 | 33.929 | 1.00 | 52.07 | C |
| ATOM | 3767 | C | VAL | B | 236 | 60.722 | 47.091 | 34.960 | 1.00 | 36.34 | C |
| ATOM | 3768 | O | VAL | B | 236 | 61.372 | 47.279 | 35.978 | 1.00 | 32.39 | O |
| ATOM | 3769 | N | VAL | B | 237 | 59.627 | 47.785 | 34.651 | 1.00 | 41.67 | N |
| ATOM | 3770 | CA | VAL | B | 237 | 58.985 | 48.731 | 35.573 | 1.00 | 38.61 | C |
| ATOM | 3771 | CB | VAL | B | 237 | 58.500 | 49.994 | 34.837 | 1.00 | 38.65 | C |
| ATOM | 3772 | CG1 | VAL | B | 237 | 58.258 | 51.109 | 35.819 | 1.00 | 36.75 | C |
| ATOM | 3773 | CG2 | VAL | B | 237 | 59.532 | 50.438 | 33.824 | 1.00 | 39.90 | C |
| ATOM | 3774 | C | VAL | B | 237 | 57.773 | 48.118 | 36.268 | 1.00 | 41.53 | C |
| ATOM | 3775 | O | VAL | B | 237 | 56.824 | 47.658 | 35.599 | 1.00 | 39.52 | O |
| ATOM | 3776 | N | GLU | B | 238 | 57.835 | 48.072 | 37.604 | 1.00 | 38.93 | N |
| ATOM | 3777 | CA | GLU | B | 238 | 56.877 | 47.288 | 38.390 | 1.00 | 41.17 | C |
| ATOM | 3778 | CB | GLU | B | 238 | 57.555 | 46.149 | 39.152 | 1.00 | 45.67 | C |
| ATOM | 3779 | CG | GLU | B | 238 | 56.564 | 45.254 | 39.882 | 1.00 | 61.77 | C |
| ATOM | 3780 | CD | GLU | B | 238 | 57.211 | 44.057 | 40.558 | 1.00 | 79.41 | C |
| ATOM | 3781 | OE1 | GLU | B | 238 | 58.444 | 44.104 | 40.810 | 1.00 | 88.21 | O |
| ATOM | 3782 | OE2 | GLU | B | 238 | 56.469 | 43.082 | 40.855 | 1.00 | 64.69 | O |
| ATOM | 3783 | C | GLU | B | 238 | 56.142 | 48.174 | 39.378 | 1.00 | 37.47 | C |
| ATOM | 3784 | O | GLU | B | 238 | 56.771 | 48.810 | 40.221 | 1.00 | 35.43 | O |
| ATOM | 3785 | N | TYR | B | 239 | 54.812 | 48.196 | 39.242 | 1.00 | 42.97 | N |
| ATOM | 3786 | CA | TYR | B | 239 | 53.890 | 48.846 | 40.180 | 1.00 | 41.04 | C |
| ATOM | 3787 | CB | TYR | B | 239 | 52.937 | 49.743 | 39.400 | 1.00 | 40.90 | C |
| ATOM | 3788 | CG | TYR | B | 239 | 53.624 | 50.882 | 38.684 | 1.00 | 37.34 | C |
| ATOM | 3789 | CD1 | TYR | B | 239 | 53.912 | 50.810 | 37.325 | 1.00 | 37.43 | C |
| ATOM | 3790 | CE1 | TYR | B | 239 | 54.517 | 51.874 | 36.668 | 1.00 | 39.05 | C |
| ATOM | 3791 | CZ | TYR | B | 239 | 54.820 | 53.025 | 37.373 | 1.00 | 22.93 | C |
| ATOM | 3792 | OH | TYR | B | 239 | 55.474 | 54.062 | 36.767 | 1.00 | 40.79 | O |
| ATOM | 3793 | CE2 | TYR | B | 239 | 54.514 | 53.133 | 38.701 | 1.00 | 7.83 | C |
| ATOM | 3794 | CD2 | TYR | B | 239 | 53.941 | 52.055 | 39.358 | 1.00 | 35.46 | C |
| ATOM | 3795 | C | TYR | B | 239 | 53.064 | 47.816 | 40.962 | 1.00 | 43.65 | C |
| ATOM | 3796 | O | TYR | B | 239 | 52.156 | 47.188 | 40.399 | 1.00 | 30.23 | O |
| ATOM | 3797 | N | LYS | B | 240 | 53.397 | 47.633 | 42.246 | 1.00 | 52.53 | N |
| ATOM | 3798 | CA | LYS | B | 240 | 52.845 | 46.529 | 43.058 | 1.00 | 56.29 | C |
| ATOM | 3799 | CB | LYS | B | 240 | 53.813 | 46.107 | 44.190 | 1.00 | 58.70 | C |
| ATOM | 3800 | CG | LYS | B | 240 | 55.212 | 45.592 | 43.759 | 1.00 | 61.52 | C |
| ATOM | 3801 | CD | LYS | B | 240 | 55.550 | 44.186 | 44.315 | 1.00 | 47.07 | C |
| ATOM | 3802 | CE | LYS | B | 240 | 56.680 | 44.225 | 45.356 | 1.00 | 66.36 | C |
| ATOM | 3803 | NZ | LYS | B | 240 | 56.621 | 43.074 | 46.307 | 1.00 | 65.86 | N |
| ATOM | 3804 | C | LYS | B | 240 | 51.504 | 46.944 | 43.669 | 1.00 | 48.47 | C |
| ATOM | 3805 | O | LYS | B | 240 | 51.428 | 47.896 | 44.448 | 1.00 | 47.69 | O |
| ATOM | 3806 | N | ILE | B | 241 | 50.437 | 46.277 | 43.257 | 1.00 | 37.63 | N |
| ATOM | 3807 | CA | ILE | B | 241 | 49.132 | 46.607 | 43.780 | 1.00 | 40.45 | C |
| ATOM | 3808 | CB | ILE | B | 241 | 48.045 | 46.064 | 42.888 | 1.00 | 41.40 | C |
| ATOM | 3809 | CG1 | ILE | B | 241 | 48.309 | 46.480 | 41.453 | 1.00 | 41.91 | C |
| ATOM | 3810 | CD1 | ILE | B | 241 | 47.506 | 45.699 | 40.434 | 1.00 | 21.64 | C |
| ATOM | 3811 | CG2 | ILE | B | 241 | 46.711 | 46.595 | 43.360 | 1.00 | 46.37 | C |
| ATOM | 3812 | C | ILE | B | 241 | 48.959 | 45.947 | 45.117 | 1.00 | 47.21 | C |
| ATOM | 3813 | O | ILE | B | 241 | 48.710 | 44.735 | 45.185 | 1.00 | 35.44 | O |
| ATOM | 3814 | N | ALA | B | 242 | 49.046 | 46.752 | 46.173 | 1.00 | 50.14 | N |
| ATOM | 3815 | CA | ALA | B | 242 | 49.173 | 46.209 | 47.518 | 1.00 | 47.48 | C |
| ATOM | 3816 | CB | ALA | B | 242 | 49.213 | 47.347 | 48.550 | 1.00 | 40.25 | C |
| ATOM | 3817 | C | ALA | B | 242 | 48.016 | 45.241 | 47.780 | 1.00 | 44.23 | C |
| ATOM | 3818 | O | ALA | B | 242 | 46.856 | 45.622 | 47.628 | 1.00 | 36.34 | O |
| ATOM | 3819 | N | ASP | B | 243 | 48.350 | 43.999 | 48.152 | 1.00 | 51.60 | N |
| ATOM | 3820 | CA | ASP | B | 243 | 47.414 | 43.018 | 48.739 | 1.00 | 52.21 | C |
| ATOM | 3821 | CB | ASP | B | 243 | 46.538 | 43.636 | 49.837 | 1.00 | 54.61 | C |
| ATOM | 3822 | CG | ASP | B | 243 | 45.829 | 42.583 | 50.661 | 1.00 | 65.28 | C |
| ATOM | 3823 | OD1 | ASP | B | 243 | 44.603 | 42.405 | 50.473 | 1.00 | 82.43 | O |
| ATOM | 3824 | OD2 | ASP | B | 243 | 46.420 | 41.840 | 51.479 | 1.00 | 76.23 | O |
| ATOM | 3825 | C | ASP | B | 243 | 46.539 | 42.355 | 47.678 | 1.00 | 51.88 | C |
| ATOM | 3826 | O | ASP | B | 243 | 45.383 | 41.995 | 47.938 | 1.00 | 56.50 | O |
| ATOM | 3827 | N | MET | B | 244 | 47.037 | 42.358 | 46.446 | 1.00 | 45.73 | N |
| ATOM | 3828 | CA | MET | B | 244 | 46.189 | 42.046 | 45.297 | 1.00 | 42.88 | C |
| ATOM | 3829 | CB | MET | B | 244 | 45.556 | 43.330 | 44.712 | 1.00 | 48.96 | C |
| ATOM | 3830 | CG | MET | B | 244 | 44.063 | 43.557 | 45.001 | 1.00 | 35.22 | C |
| ATOM | 3831 | SD | MET | B | 244 | 43.102 | 42.081 | 44.660 | 1.00 | 68.19 | S |
| ATOM | 3832 | CE | MET | B | 244 | 42.295 | 42.506 | 43.088 | 1.00 | 70.55 | C |
| ATOM | 3833 | C | MET | B | 244 | 47.104 | 41.392 | 44.262 | 1.00 | 46.17 | C |
| ATOM | 3834 | O | MET | B | 244 | 46.760 | 40.354 | 43.672 | 1.00 | 25.17 | O |
| ATOM | 3835 | N | GLY | B | 245 | 48.252 | 42.041 | 44.019 | 1.00 | 49.32 | N |
| ATOM | 3836 | CA | GLY | B | 245 | 49.096 | 41.719 | 42.858 | 1.00 | 38.97 | C |
| ATOM | 3837 | C | GLY | B | 245 | 50.081 | 42.811 | 42.485 | 1.00 | 38.57 | C |
| ATOM | 3838 | O | GLY | B | 245 | 50.580 | 43.571 | 43.333 | 1.00 | 40.78 | O |
| ATOM | 3839 | N | HIS | B | 246 | 50.483 | 42.789 | 41.216 | 1.00 | 36.26 | N |
| ATOM | 3840 | CA | HIS | B | 246 | 51.506 | 43.718 | 40.743 | 1.00 | 39.57 | C |
| ATOM | 3841 | CB | HIS | B | 246 | 52.862 | 43.043 | 40.814 | 1.00 | 27.89 | C |
| ATOM | 3842 | CG | HIS | B | 246 | 52.959 | 41.863 | 39.903 | 1.00 | 40.41 | C |
| ATOM | 3843 | ND1 | HIS | B | 246 | 52.493 | 40.615 | 40.255 | 1.00 | 67.51 | N |
| ATOM | 3844 | CE1 | HIS | B | 246 | 52.668 | 39.779 | 39.241 | 1.00 | 77.61 | C |
| ATOM | 3845 | NE2 | HIS | B | 246 | 53.190 | 40.449 | 38.227 | 1.00 | 59.35 | N |
| ATOM | 3846 | CD2 | HIS | B | 246 | 53.330 | 41.767 | 38.598 | 1.00 | 65.46 | C |
| ATOM | 3847 | C | HIS | B | 246 | 51.204 | 44.073 | 39.293 | 1.00 | 43.06 | C |
| ATOM | 3048 | O | HIS | B | 246 | 50.467 | 43.369 | 38.603 | 1.00 | 51.40 | O |
| ATOM | 3049 | N | LEU | B | 247 | 51.767 | 45.171 | 38.819 | 1.00 | 40.69 | N |
| ATOM | 3850 | CA | LEU | B | 247 | 51.593 | 45.477 | 37.429 | 1.00 | 29.79 | C |
| ATOM | 3851 | CB | LEU | B | 247 | 50.740 | 46.727 | 37.317 | 1.00 | 33.34 | C |
| ATOM | 3852 | CG | LHU | B | 247 | 50.161 | 47.045 | 35.931 | 1.00 | 49.73 | C |
| ATOM | 3853 | CD1 | LEU | B | 247 | 49.637 | 45.830 | 35.188 | 1.00 | 38.99 | C |
| ATOM | 3854 | CD2 | LEU | B | 247 | 49.115 | 48.148 | 36.016 | 1.00 | 28.10 | C |
| ATOM | 3855 | C | LEU | B | 247 | 52.981 | 45.676 | 36.870 | 1.00 | 28.97 | C |
| ATOM | 3856 | O | LEU | B | 247 | 53.644 | 46.659 | 37.207 | 1.00 | 35.14 | O |
| ATOM | 3057 | N | LYS | B | 248 | 53.427 | 44.739 | 36.038 | 1.00 | 27.55 | N |
| ATOM | 3858 | CA | LYS | B | 248 | 54.759 | 44.803 | 35.447 | 1.00 | 33.64 | C |
| ATOM | 3859 | CB | LYS | B | 248 | 55.389 | 43.427 | 35.475 | 1.00 | 37.26 | C |
| ATOM | 3860 | CG | LYS | B | 248 | 56.407 | 43.255 | 36.597 | 1.00 | 52.53 | C |
| ATOM | 3861 | CD | LYS | B | 248 | 57.073 | 41.886 | 36.499 | 1.00 | 55.58 | C |
| ATOM | 3862 | CE | LYS | B | 248 | 57.130 | 41.182 | 37.849 | 1.00 | 55.07 | C |
| ATOM | 3863 | NZ | LYS | B | 248 | 56.728 | 39.739 | 37.767 | 1.00 | 63.94 | N |
| ATOM | 3864 | C | LYS | B | 248 | 54.791 | 45.340 | 34.010 | 1.00 | 36.38 | C |
| ATOM | 3865 | O | LYS | B | 248 | 53.883 | 45.061 | 33.215 | 1.00 | 33.21 | O |
| ATOM | 3866 | N | TYR | B | 249 | 55.813 | 46.147 | 33.706 | 1.00 | 34.70 | N |
| ATOM | 3867 | CA | TYR | B | 249 | 55.900 | 46.855 | 32.431 | 1.00 | 30.01 | C |
| ATOM | 3868 | CB | TYR | B | 249 | 55.717 | 48.366 | 32.658 | 1.00 | 23.10 | C |
| ATOM | 3869 | CG | TYR | B | 249 | 54.283 | 48.843 | 32.694 | 1.00 | 17.92 | C |
| ATOM | 3870 | CD1 | TYR | B | 249 | 53.434 | 48.633 | 31.618 | 1.00 | 31.54 | C |
| ATOM | 3871 | CE1 | TYR | B | 249 | 52.129 | 49.068 | 31.646 | 1.00 | 38.16 | C |
| ATOM | 3872 | CZ | TYR | B | 249 | 51.659 | 49.749 | 32.759 | 1.00 | 60.20 | C |
| ATOM | 3873 | OH | TYR | B | 249 | 50.331 | 50.151 | 32.810 | 1.00 | 69.37 | O |
| ATOM | 3874 | CE2 | TYR | B | 249 | 52.506 | 50.001 | 33.824 | 1.00 | 46.88 | C |
| ATOM | 3875 | CD2 | TYR | B | 249 | 53.801 | 49.552 | 33.782 | 1.00 | 39.20 | C |
| ATOM | 3876 | C | TYR | B | 249 | 57.281 | 46.600 | 31.834 | 1.00 | 33.56 | C |
| ATOM | 3077 | O | TYR | B | 249 | 58.256 | 47.240 | 32.218 | 1.00 | 36.60 | O |
| ATOM | 3878 | N | TYR | B | 250 | 57.373 | 45.679 | 30.886 | 1.00 | 29.52 | N |
| ATOM | 3879 | CA | TYR | B | 250 | 50.661 | 45.411 | 30.250 | 1.00 | 32.48 | C |
| ATOM | 3880 | CB | TYR | B | 250 | 58.695 | 43.976 | 29.747 | 1.00 | 37.85 | C |
| ATOM | 3881 | CG | TYR | B | 250 | 58.597 | 42.938 | 30.840 | 1.00 | 52.48 | C |
| ATOM | 3882 | CD1 | TYR | B | 250 | 57.368 | 42.597 | 31.384 | 1.00 | 44.77 | C |
| ATOM | 3883 | CE1 | TYR | B | 250 | 57.263 | 41.650 | 32.394 | 1.00 | 33.56 | C |
| ATOM | 3884 | CZ | TYR | B | 250 | 58.376 | 41.007 | 32.841 | 1.00 | 48.27 | C |
| ATOM | 3885 | OH | TYR | B | 250 | 58.198 | 40.054 | 33.814 | 1.00 | 61.25 | O |
| ATOM | 3886 | CE2 | TYR | B | 250 | 59.622 | 41.322 | 32.319 | 1.00 | 65.81 | C |
| ATOM | 3887 | CD2 | TYR | B | 250 | 59.729 | 42.291 | 31.326 | 1.00 | 63.42 | C |
| ATOM | 3888 | C | TYR | B | 250 | 58.914 | 46.334 | 29.070 | 1.00 | 36.04 | C |
| ATOM | 3889 | O | TYR | B | 250 | 57.975 | 46.672 | 28.335 | 1.00 | 38.98 | O |
| ATOM | 3890 | N | LEU | B | 251 | 60.174 | 46.712 | 28.854 | 1.00 | 32.98 | N |
| ATOM | 3891 | CA | LEU | B | 251 | 60.481 | 47.671 | 27.791 | 1.00 | 31.30 | C |
| ATOM | 3892 | CB | LEU | B | 251 | 60.429 | 49.108 | 28.287 | 1.00 | 35.45 | C |
| ATOM | 3893 | CG | LEU | B | 251 | 60.771 | 50.184 | 27.239 | 1.00 | 37.78 | C |
| ATOM | 3894 | CD1 | LEU | B | 251 | 59.741 | 50.115 | 26.106 | 1.00 | 24.16 | C |
| ATOM | 3895 | CD2 | LEU | B | 251 | 60.834 | 51.615 | 27.864 | 1.00 | 26.76 | C |
| ATOM | 3896 | C | LEU | B | 251 | 61.848 | 47.464 | 27.244 | 1.00 | 29.73 | C |
| ATOM | 3897 | O | LEU | B | 251 | 62.804 | 47.462 | 27.997 | 1.00 | 36.28 | O |
| ATOM | 3898 | N | ALA | B | 252 | 61.955 | 47.411 | 25.927 | 1.00 | 33.59 | N |
| ATOM | 3899 | CA | ALA | B | 252 | 63.213 | 47.061 | 25.286 | 1.00 | 35.65 | C |
| ATOM | 3900 | CB | ALA | B | 252 | 62.950 | 46.627 | 23.869 | 1.00 | 47.87 | C |
| ATOM | 3901 | C | ALA | B | 252 | 64.143 | 48.255 | 25.297 | 1.00 | 37.36 | C |
| ATOM | 3902 | O | ALA | B | 252 | 63.694 | 49.389 | 25.157 | 1.00 | 49.04 | O |
| ATOM | 3903 | N | PRO | B | 253 | 65.443 | 48.002 | 25.396 | 1.00 | 35.04 | N |
| ATOM | 3904 | CA | PRO | B | 253 | 66.468 | 49.022 | 25.137 | 1.00 | 31.43 | C |
| ATOM | 3905 | CB | PRO | B | 253 | 67.765 | 48.250 | 25.341 | 1.00 | 27.30 | C |
| ATOM | 3906 | CG | PRO | B | 253 | 67.394 | 47.134 | 26.218 | 1.00 | 33.01 | C |
| ATOM | 3907 | CD | PRO | B | 253 | 66.023 | 46.716 | 25.803 | 1.00 | 34.13 | C |
| ATOM | 3908 | C | PRO | B | 253 | 66.433 | 49.515 | 23.701 | 1.00 | 36.06 | C |
| ATOM | 3909 | O | PRO | B | 253 | 65.918 | 48.813 | 22.824 | 1.00 | 49.02 | O |
| ATOM | 3910 | N | LYS | B | 254 | 67.047 | 50.662 | 23.444 | 1.00 | 32.12 | N |
| ATOM | 3911 | CA | LYS | B | 254 | 67.198 | 51.134 | 22.090 | 1.00 | 36.48 | C |
| ATOM | 3912 | CB | LYS | B | 254 | 67.263 | 52.648 | 22.083 | 1.00 | 24.55 | C |
| ATOM | 3913 | CG | LYS | B | 254 | 66.172 | 53.258 | 21.224 | 1.00 | 49.23 | C |
| ATOM | 3914 | CD | LYS | B | 254 | 65.534 | 54.477 | 21.864 | 1.00 | 33.00 | C |
| ATOM | 3915 | CE | LYS | B | 254 | 65.357 | 55.641 | 20.863 | 1.00 | 50.56 | C |
| ATOM | 3916 | NZ | LYS | B | 254 | 64.035 | 56.344 | 21.020 | 1.00 | 63.48 | N |
| ATOM | 3917 | C | LYS | B | 254 | 68.416 | 50.498 | 21.390 | 1.00 | 55.27 | C |
| ATOM | 3918 | O | LYS | B | 254 | 68.721 | 49.296 | 21.553 | 1.00 | 60.17 | O |
| ATOM | 3919 | N | ILE | B | 255 | 69.019 | 51.260 | 20.478 | 1.00 | 63.83 | N |
| ATOM | 3920 | CA | ILE | B | 255 | 69.782 | 50.663 | 19.372 | 1.00 | 59.47 | C |
| ATOM | 3921 | CB | ILE | B | 255 | 70.440 | 51.824 | 18.553 | 1.00 | 61.54 | C |
| ATOM | 3922 | CG1 | ILE | B | 255 | 69.452 | 52.342 | 17.491 | 1.00 | 71.86 | C |
| ATOM | 3923 | CD1 | ILE | B | 255 | 68.846 | 53.715 | 17.764 | 1.00 | 66.16 | C |
| ATOM | 3924 | CG2 | ILE | B | 255 | 71.701 | 51.357 | 17.817 | 1.00 | 78.61 | C |
| ATOM | 3925 | C | ILE | B | 255 | 70.822 | 49.715 | 19.989 | 1.00 | 52.41 | C |
| ATOM | 3926 | O | ILE | B | 255 | 71.498 | 50.116 | 20.962 | 1.00 | 62.81 | O |
| ATOM | 3927 | OW0 | HOH | W | 2 | 36.755 | −2.242 | 12.588 | 1.00 | 30.73 | O |
| ATOM | 3928 | OW0 | HOH | W | 5 | 40.086 | −2.577 | 20.226 | 1.00 | 39.11 | O |
| ATOM | 3929 | OW0 | HOH | W | 7 | 20.256 | −16.089 | −2.898 | 1.00 | 37.46 | O |
| ATOM | 3930 | OW0 | HOH | W | 11 | 61.274 | 55.246 | 48.100 | 1.00 | 35.60 | O |
| ATOM | 3931 | OW0 | HOH | W | 12 | 33.935 | 58.947 | 41.499 | 1.00 | 38.91 | O |
| ATOM | 3932 | OW0 | HOH | W | 13 | 51.095 | 57.446 | 43.775 | 1.00 | 35.23 | O |
| ATOM | 3933 | OW0 | HOH | W | 14 | 67.063 | 45.801 | 31.342 | 1.00 | 43.55 | O |
| ATOM | 3934 | OW0 | HOH | W | 15 | 33.986 | 8.241 | 8.375 | 1.00 | 38.92 | O |
| ATOM | 3935 | OW0 | HOH | W | 16 | 24.350 | 17.901 | −0.621 | 1.00 | 43.54 | O |
| ATOM | 3936 | OW0 | HOH | W | 18 | 24.952 | 43.195 | 32.621 | 1.00 | 47.67 | O |
| ATOM | 3937 | OW0 | HOH | W | 20 | 18.950 | 27.240 | 1.282 | 1.00 | 38.98 | O |
| ATOM | 3930 | OW0 | HOH | W | 21 | 28.753 | 1.214 | −20.795 | 1.00 | 54.90 | O |
| ATOM | 3939 | OW0 | HOH | W | 22 | 65.060 | 67.827 | 19.602 | 1.00 | 43.98 | O |
| ATOM | 3940 | OW0 | HOH | W | 23 | 72.781 | 64.459 | 28.563 | 1.00 | 38.64 | O |
| ATOM | 3941 | OW0 | HOH | W | 24 | 53.306 | 40.808 | 44.223 | 1.00 | 30.87 | O |
| ATOM | 3942 | OW0 | HOH | W | 25 | 56.208 | 38.093 | 32.561 | 1.00 | 66.37 | O |
| ATOM | 3943 | OW0 | HOH | W | 30 | 74.956 | 58.023 | 47.246 | 1.00 | 46.10 | O |
| ATOM | 3944 | OW0 | HOH | W | 31 | 24.747 | 20.956 | −14.832 | 1.00 | 34.44 | O |
| ATOM | 3945 | OW0 | HOH | W | 32 | 40.894 | 35.639 | 16.828 | 1.00 | 45.78 | O |
| ATOM | 3946 | OW0 | HOH | W | 33 | 48.548 | 36.083 | 32.049 | 1.00 | 68.75 | O |
| ATOM | 3947 | OW0 | HOH | W | 34 | 9.942 | 31.486 | 8.328 | 1.00 | 53.47 | O |
| ATOM | 3948 | OW0 | HOH | W | 35 | 46.529 | −11.949 | 11.821 | 1.00 | 40.92 | O |
| ATOM | 3949 | OW0 | HOH | W | 36 | 49.450 | 40.818 | 48.569 | 1.00 | 51.89 | O |
| ATOM | 3950 | OW0 | HOH | W | 37 | 29.762 | −10.201 | 10.020 | 1.00 | 42.15 | O |
| ATOM | 3951 | OW0 | HOH | W | 39 | 27.299 | 15.954 | 2.069 | 1.00 | 56.59 | O |
| ATOM | 3952 | OW0 | HOH | W | 40 | 53.358 | 37.217 | 37.509 | 1.00 | 47.45 | O |
| ATOM | 3953 | OW0 | HOH | W | 41 | 4.129 | 3.482 | −6.107 | 1.00 | 38.21 | O |
| ATOM | 3954 | OW0 | HOH | W | 44 | 76.134 | 46.455 | 31.991 | 1.00 | 49.76 | O |
| ATOM | 3955 | OW0 | HOH | W | 45 | 8.189 | 24.331 | −3.018 | 1.00 | 59.82 | O |
| ATOM | 3956 | OW0 | HOH | W | 46 | 25.727 | 40.302 | 42.085 | 1.00 | 46.74 | O |
| ATOM | 3957 | OW0 | HOH | W | 47 | 71.902 | 59.445 | 41.327 | 1.00 | 72.67 | O |
| ATOM | 3958 | OW0 | HOH | W | 50 | 57.449 | 66.359 | 41.256 | 1.00 | 52.30 | O |
| ATOM | 3959 | OW0 | HOH | W | 52 | 42.341 | 32.562 | 12.794 | 1.00 | 54.67 | O |
| ATOM | 3960 | OW0 | HOH | W | 53 | 8.498 | 26.892 | 9.531 | 1.00 | 44.09 | O |
| ATOM | 3961 | OW0 | HOH | W | 54 | 30.827 | 13.766 | −3.930 | 1.00 | 55.38 | O |
| ATOM | 3962 | OW0 | HOH | W | 55 | 37.326 | 25.993 | 30.921 | 1.00 | 47.80 | O |
| ATOM | 3963 | OW0 | HOH | W | 57 | 65.994 | 33.490 | 33.135 | 1.00 | 40.47 | O |
| ATOM | 3964 | OW0 | HOH | W | 58 | 48.794 | 11.076 | 5.616 | 1.00 | 42.24 | O |
| ATOM | 3965 | OW0 | HOH | W | 60 | 46.455 | 46.589 | 21.124 | 1.00 | 42.75 | O |
| ATOM | 3966 | OW0 | HOH | W | 61 | 41.403 | −16.036 | 14.606 | 1.00 | 51.36 | O |
| ATOM | 3967 | OW0 | HOH | W | 63 | 70.947 | 53.275 | 48.454 | 1.00 | 47.97 | O |
| ATOM | 3968 | OW0 | HOH | W | 64 | 44.567 | 7.394 | 5.984 | 1.00 | 39.80 | O |
| ATOM | 3969 | OW0 | HOH | W | 65 | 23.204 | −18.705 | 10.039 | 1.00 | 50.46 | O |
| ATOM | 3970 | OW0 | HOH | W | 67 | 39.633 | 52.444 | 20.828 | 1.00 | 45.13 | O |
| ATOM | 3971 | OW0 | HOH | W | 69 | 31.524 | 56.168 | 45.217 | 1.00 | 68.71 | O |
| ATOM | 3972 | OW0 | HOH | W | 70 | 45.024 | 57.198 | 31.417 | 1.00 | 47.40 | O |
| ATOM | 3973 | OW0 | HOH | W | 71 | 9.389 | 20.161 | 16.994 | 1.00 | 41.90 | O |
| ATOM | 3974 | OW0 | HOH | W | 75 | 43.897 | 6.107 | −11.499 | 1.00 | 58.72 | O |
| ATOM | 3975 | OW0 | HOH | W | 77 | 39.371 | 44.427 | 17.132 | 1.00 | 50.18 | O |
| ATOM | 3976 | OW0 | HOH | W | 78 | 40.333 | 49.711 | 20.723 | 1.00 | 54.37 | O |
| ATOM | 3977 | OW0 | HOH | W | 79 | 38.914 | 36.274 | 22.496 | 1.00 | 57.88 | O |
| ATOM | 3978 | OW0 | HOH | W | 81 | 28.027 | 9.318 | −14.052 | 1.00 | 40.77 | O |
| ATOM | 3979 | OW0 | HOH | W | 82 | 27.521 | 55.148 | 49.379 | 1.00 | 64.01 | O |
| ATOM | 3980 | OW0 | HOH | W | 85 | 19.025 | 33.523 | 14.107 | 1.00 | 44.74 | O |
| ATOM | 3981 | OW0 | HOH | W | 87 | 41.273 | −13.395 | −8.666 | 1.00 | 37.00 | O |
| ATOM | 3982 | OW0 | HOH | W | 88 | 33.788 | −12.272 | −12.851 | 1.00 | 65.97 | O |
| ATOM | 3983 | OW0 | HOH | W | 89 | 56.644 | 62.788 | 50.727 | 1.00 | 75.79 | O |
| ATOM | 3984 | OW0 | HOH | W | 90 | 8.175 | 19.945 | 8.731 | 1.00 | 54.81 | O |

TABLE 5
Coordinates of the complex of human PCNA_CM peptide complex of space group
P3₁21 with unit dimensions: a = 119.1 Å, b = 119.1 Å, c = 305.82 Å

| | | | |
|---|---|---|---|
| REMARK | 3 | REFINEMENT. | |
| REMARK | 3 | PROGRAM | : REFMAC 5.1.24 |
| REMARK | 3 | AUTHORS | : MURSHUDOV, VAGIN, DODSON |
| REMARK | 3 | | |

| REMARK | 3 | REFINEMENT TARGET : MAXIMUM LIKELIHOOD | | |
|---|---|---|---|---|
| REMARK | 3 | | | |
| REMARK | 3 | DATA USED IN REFINEMENT. | | |
| REMARK | 3 | RESOLUTION RANGE HIGH | (ANGSTROMS) : | 2.80 |
| REMARK | 3 | RESOLUTION RANGE LOW | (ANGSTROMS) : | 13.99 |
| REMARK | 3 | DATA CUTOFF | (SIGMA(F)) : | NONE |

| | | | | |
|---|---|---|---|---|
| REMARK | 3 | COMPLETENESS FOR RANGE | (%) : | 99.52 |
| REMARK | 3 | NUMBER OF REFLECTIONS : | 60068 | |
| REMARK | 3 | | | |

| REMARK | 3 | FIT TO DATA USED IN REFINEMENT. | | |
|---|---|---|---|---|
| REMARK | 3 | CROSS-VALIDATION METHOD | | : THROUGHOUT |
| REMARK | 3 | FREE R VALUE TEST SET SELECTION | | : RANDOM |
| REMARK | 3 | R VALUE | (WORKING + TEST SET) | : 0.17897 |
| REMARK | 3 | R VALUE | (WORKING SET) | : 0.17644 |

| REMARK | 3 | FREE R VALUE | | : 0.25595 |
|---|---|---|---|---|
| REMARK | 3 | FREE R VALUE TEST SET SIZE | (%) | : 3.1 |

| | | | |
|---|---|---|---|
| REMARK | 3 | FREE R VALUE TEST SET COUNT | : 1917 |
| REMARK | 3 | | |

| REMARK | 3 | FIT IN THE HIGHEST RESOLUTION BIN. | | |
|---|---|---|---|---|
| REMARK | 3 | TOTAL NUMBER OF BINS USED | | : 20 |
| REMARK | 3 | BIN RESOLUTION RANGE HIGH | | : 2.800 |
| REMARK | 3 | BIN RESOLUTION RANGE LOW | | : 2.870 |
| REMARK | 3 | REFLECTION IN BIN | (WORKING SET) | : 4279 |
| REMARK | 3 | BIN R VALUE | (WORKING SET) | : 0.309 |

| | | | |
|---|---|---|---|
| REMARK | 3 | BIN FREE R VALUE SET COUNT | : 129 |
| REMARK | 3 | BIN FREE R VALUE | : 0.416 |
| REMARK | 3 | | |

| REMARK | 3 | NUMBER OF NON-HYDROGEN ATOMS USED IN REFINEMENT. | |
|---|---|---|---|
| REMARK | 3 | ALL ATOMS | : 13065 |
| REMARK | 3 | | |
| REMARK | 3 | B VALUES. | |

| | | | | |
|---|---|---|---|---|
| REMARK | 3 | FROM WILSON PLOT | (A**2) | : NULL |
| REMARK | 3 | MEAN B VALUE | (OVERALL, A**2) | : 54.299 |

| REMARK | 3 | OVERALL ANISOTROPIC B VALUE. | | |
|---|---|---|---|---|
| REMARK | 3 | B11 (A**2) : −0.01 | | |
| REMARK | 3 | B22 (A**2) : −0.01 | | |
| REMARK | 3 | B33 (A**2) : 0.01 | | |
| REMARK | 3 | B12 (A**2) : 0.00 | | |
| REMARK | 3 | B13 (A**2) : 0.00 | | |
| REMARK | 3 | B23 (A**2) : 0.00 | | |
| REMARK | 3 | | | |
| REMARK | 3 | ESTIMATED OVERALL COORDINATE ERROR. | | |
| REMARK | 3 | ESU BASED ON R VALUE | (A) : | 0.647 |
| REMARK | 3 | ESU BASED ON FREE R VALUE | (A) : | 0.338 |
| REMARK | 3 | ESU BASED ON MAXIMUM LIKELIHOOD | (A) : | 0.255 |
| REMARK | 3 | ESU FOR B VALUES BASED ON MAXIMUM LIKELIHOOD | (A**2) : | 13.207 |
| REMARK | 3 | | | |
| REMARK | 3 | CORRELATION COEFFICIENTS. | | |

| REMARK | 3 | CORRELATION COEFFICIENT FO-FC | | : 0.954 | | |
|---|---|---|---|---|---|---|
| REMARK | 3 | CORRELATION COEFFICIENT FO-FC FREE | | : 0.907 | | |
| REMARK | 3 | | | | | |
| REMARK | 3 | RMS DEVIATIONS FROM IDEAL VALUES | | COUNT | RMS | WEIGHT |
| REMARK | 3 | BOND LENGTHS REFINED ATOMS | (A): | 12917 ; | 0.015 ; | 0.022 |
| REMARK | 3 | BOND LENGTHS OTHERS | (A): | 11826 ; | 0.001 ; | 0.020 |
| REMARK | 3 | BOND ANGLES REFINED ATOMS | (DEGREES): | 17434 ; | 2.066 ; | 1.978 |
| REMARK | 3 | BOND ANGLES OTHERS | (DEGREES): | 27712 ; | 0.891 ; | 3.000 |
| REMARK | 3 | TORSION ANGLES, PERIOD 1 | (DEGREES): | 1633 ; | 12.568 ; | 5.000 |
| REMARK | 3 | CHIRAL-CENTER RESTRAINTS | (A**3): | 2046 ; | 0.128 ; | 0.200 |

| | | | | | | |
|---|---|---|---|---|---|---|
| REMARK | 3 | GENERAL PLANES REFINED ATOMS | (A): | 14189 ; | 0.010 ; | 0.020 |
| REMARK | 3 | GENERAL PLANES OTHERS | (A): | 2317 ; | 0.006 ; | 0.020 |
| REMARK | 3 | NON-BONDED CONTACTS REFINED ATOMS | (A): | 2639 ; | 0.266 ; | 0.300 |
| REMARK | 3 | NON-BONDED CONTACTS OTHERS | (A): | 14577 ; | 0.282 ; | 0.300 |
| REMARK | 3 | NON-BONDED TORSION OTHERS | (A): | 9234 ; | 0.109 ; | 0.500 |
| REMARK | 3 | H-BOND (X . . . Y) REFINED ATOMS | (A): | 724 ; | 0.232 ; | 0.500 |
| REMARK | 3 | SYMMETRY VDW REFINED ATOMS | (A): | 29 ; | 0.267 ; | 0.300 |
| REMARK | 3 | SYMMETRY VDW OTHERS | (A): | 75 ; | 0.287 ; | 0.300 |
| REMARK | 3 | SYMMETRY H-BOND REFINED ATOMS | (A): | 13 ; | 0.324 ; | 0.500 |
| REMARK | 3 | | | | | |

| REMARK | 3 | ISOTROPIC THERMAL FACTOR RESTRAINTS. | | COUNT | RMS | WEIGHT |
|---|---|---|---|---|---|---|
| REMARK | 3 | MAIN-CHAIN BOND REFINED ATOMS | (A**2): | 8184 ; | 5.090 ; | 1.500 |
| REMARK | 3 | MAIN-CHAIN ANGLE REFINED ATOMS | (A**2): | 13213 ; | 8.031 ; | 2.000 |
| REMARK | 3 | SIDE-CHAIN BOND REFINED ATOMS | (A**2): | 4733 ; | 11.795 ; | 3.000 |
| REMARK | 3 | SIDE-CHAIN ANGLE REFINED ATOMS | (A**2): | 4221 ; | 16.674 ; | 4.500 |
| REMARK | 3 | | | | | |

| REMARK | 3 | NCS RESTRAINTS STATISTICS | | | |
|---|---|---|---|---|---|
| REMARK | 3 | NUMBER OF NCS GROUPS : NULL | | | |
| REMARK | 3 | | | | |
| REMARK | 3 | | | | |
| REMARK | 3 | TLS DETAILS | | | |
| REMARK | 3 | NUMBER OF TLS GROUPS : NULL | | | |
| REMARK | 3 | | | | |
| REMARK | 3 | | | | |
| REMARK | 3 | BULK SOLVENT MODELLING. | | | |
| REMARK | 3 | METHOD USED: BABINET MODEL WITH MASK | | | |
| REMARK | 3 | PARAMETERS FOR MASK CALCULATION | | | |
| REMARK | 3 | VDW PROBE RADIUS | | | : 1.40 |
| REMARK | 3 | ION PROBE RADIUS | | | : 0.80 |
| REMARK | 3 | SHRINKAGE RADIUS | | | : 0.80 |
| REMARK | 3 | | | | |
| REMARK | 3 | OTHER REFINEMENT REMARKS: | | | |
| REMARK | 3 | HYDROGENS HAVE BEEN ADDED IN THE RIDING POSITIONS | | | |
| REMARK | 3 | | | | |
| SSBOND | 1 | CYS C | 135 | CYS C | 162 |
| SSBOND | 2 | CYS E | 135 | CYS E | 162 |
| SSBOND | 3 | CYS I | 135 | CYS I | 162 |
| SSBOND | 4 | CYS K | 135 | CYS K | 162 |
| SSBOND | 5 | CYS G | 135 | CYS G | 162 |

| | |
|---|---|
| CRYST1 | 119.101 119.101 305.817 90.00 90.00 120.00 P 31 2 1 |

| | |
|---|---|
| SCALE1 | 0.008396 0.004848 0.000000 0.00000 |
| SCALE2 | 0.000000 0.009695 0.000000 0.00000 |
| SCALE3 | 0.000000 0.000000 0.003270 0.00000 |

| | | | | | | | | | | | |
|---|---|---|---|---|---|---|---|---|---|---|---|
| ATOM | 1 | N | MET | A | 1 | −46.103 | 83.341 | 3.259 | 1.00 | 68.66 | N |
| ATOM | 3 | CA | MET | A | 1 | −44.819 | 83.180 | 2.517 | 1.00 | 67.65 | C |
| ATOM | 5 | CB | MET | A | 1 | −44.751 | 84.242 | 1.428 | 1.00 | 72.44 | C |
| ATOM | 8 | CG | MET | A | 1 | −43.970 | 83.824 | 0.184 | 1.00 | 90.61 | C |
| ATOM | 11 | SD | MET | A | 1 | −42.244 | 83.335 | 0.465 | 1.00 | 102.36 | S |
| ATOM | 12 | CE | MET | A | 1 | −42.125 | 81.989 | −0.775 | 1.00 | 88.06 | C |
| ATOM | 16 | C | MET | A | 1 | −43.627 | 83.382 | 3.442 | 1.00 | 62.28 | C |
| ATOM | 17 | O | MET | A | 1 | −43.437 | 84.520 | 3.892 | 1.00 | 59.88 | O |
| ATOM | 20 | N | PHE | A | 2 | −42.859 | 82.311 | 3.726 | 1.00 | 53.48 | N |
| ATOM | 22 | CA | PHE | A | 2 | −41.810 | 82.311 | 4.774 | 1.00 | 43.59 | C |
| ATOM | 24 | CB | PHE | A | 2 | −41.977 | 81.133 | 5.740 | 1.00 | 42.61 | C |
| ATOM | 27 | CG | PHE | A | 2 | −40.742 | 80.796 | 6.536 | 1.00 | 39.08 | C |
| ATOM | 28 | CD1 | PHE | A | 2 | −40.166 | 81.722 | 7.387 | 1.00 | 51.11 | C |
| ATOM | 30 | CE1 | PHE | A | 2 | −38.993 | 81.411 | 8.115 | 1.00 | 50.15 | C |
| ATOM | 32 | CZ | PHE | A | 2 | −38.379 | 80.191 | 7.959 | 1.00 | 26.02 | C |
| ATOM | 34 | CE2 | PHE | A | 2 | −38.978 | 79.234 | 7.162 | 1.00 | 43.37 | C |
| ATOM | 36 | CD2 | PHE | A | 2 | −40.141 | 79.544 | 6.435 | 1.00 | 51.03 | C |
| ATOM | 38 | C | PHE | A | 2 | −40.497 | 82.157 | 4.069 | 1.00 | 41.28 | C |
| ATOM | 39 | O | PHE | A | 2 | −40.390 | 81.232 | 3.275 | 1.00 | 54.52 | O |
| ATOM | 40 | N | GLU | A | 3 | −39.499 | 82.993 | 4.345 | 1.00 | 35.58 | N |
| ATOM | 42 | CA | GLU | A | 3 | −38.146 | 82.765 | 3.762 | 1.00 | 49.13 | C |
| ATOM | 44 | CB | GLU | A | 3 | −37.949 | 83.525 | 2.424 | 1.00 | 56.21 | C |
| ATOM | 47 | CG | GLU | A | 3 | −36.632 | 83.333 | 1.655 | 1.00 | 64.37 | C |
| ATOM | 50 | CD | GLU | A | 3 | −36.767 | 83.305 | 0.124 | 1.00 | 64.41 | C |
| ATOM | 51 | OE1 | GLU | A | 3 | −36.027 | 82.553 | −0.535 | 1.00 | 66.05 | O |
| ATOM | 52 | OE2 | GLU | A | 3 | −37.610 | 84.018 | −0.460 | 1.00 | 74.26 | O |
| ATOM | 53 | C | GLU | A | 3 | −37.033 | 83.157 | 4.743 | 1.00 | 45.78 | C |
| ATOM | 54 | O | GLU | A | 3 | −37.007 | 84.263 | 5.274 | 1.00 | 49.35 | O |
| ATOM | 55 | N | ALA | A | 4 | −36.109 | 82.249 | 5.008 | 1.00 | 42.97 | N |
| ATOM | 57 | CA | ALA | A | 4 | −35.045 | 82.537 | 5.966 | 1.00 | 46.91 | C |
| ATOM | 59 | CB | ALA | A | 4 | −35.303 | 81.806 | 7.291 | 1.00 | 44.34 | C |
| ATOM | 63 | C | ALA | A | 4 | −33.747 | 82.066 | 5.326 | 1.00 | 49.37 | C |
| ATOM | 64 | O | ALA | A | 4 | −33.654 | 80.907 | 4.938 | 1.00 | 60.15 | O |
| ATOM | 65 | N | ARG | A | 5 | −32.771 | 82.941 | 5.125 | 1.00 | 45.50 | N |
| ATOM | 67 | CA | ARG | A | 5 | −31.461 | 82.447 | 4.704 | 1.00 | 44.65 | C |
| ATOM | 69 | CB | ARG | A | 5 | −31.008 | 83.063 | 3.375 | 1.00 | 46.17 | C |
| ATOM | 72 | CG | ARG | A | 5 | −29.922 | 84.111 | 3.508 | 1.00 | 54.45 | C |
| ATOM | 75 | CD | ARG | A | 5 | −28.960 | 84.237 | 2.295 | 1.00 | 68.24 | C |
| ATOM | 78 | NE | ARG | A | 5 | −29.641 | 84.639 | 1.068 | 1.00 | 44.96 | N |
| ATOM | 80 | CZ | ARG | A | 5 | −29.792 | 83.862 | 0.014 | 1.00 | 53.29 | C |
| ATOM | 81 | NH1 | ARG | A | 5 | −29.235 | 82.661 | −0.077 | 1.00 | 85.20 | N |
| ATOM | 84 | NH2 | ARG | A | 5 | −30.466 | 84.315 | −1.007 | 1.00 | 45.74 | N |
| ATOM | 87 | C | ARG | A | 5 | −30.391 | 82.630 | 5.765 | 1.00 | 37.81 | C |
| ATOM | 88 | O | ARG | A | 5 | −30.399 | 83.651 | 6.448 | 1.00 | 33.58 | O |
| ATOM | 89 | N | LEU | A | 6 | −29.492 | 81.637 | 5.856 | 1.00 | 38.40 | N |
| ATOM | 91 | CA | LEU | A | 6 | −28.263 | 81.675 | 6.660 | 1.00 | 44.56 | C |
| ATOM | 93 | CB | LEU | A | 6 | −28.265 | 80.546 | 7.688 | 1.00 | 43.20 | C |
| ATOM | 96 | CG | LEU | A | 6 | −28.717 | 80.920 | 9.093 | 1.00 | 53.51 | C |
| ATOM | 98 | CD1 | LEU | A | 6 | −29.020 | 79.640 | 9.852 | 1.00 | 53.63 | C |
| ATOM | 102 | CD2 | LEU | A | 6 | −27.658 | 81.734 | 9.795 | 1.00 | 57.61 | C |
| ATOM | 106 | C | LEU | A | 6 | −26.979 | 81.540 | 5.836 | 1.00 | 45.45 | C |
| ATOM | 107 | O | LEU | A | 6 | −26.738 | 80.534 | 5.154 | 1.00 | 45.22 | O |
| ATOM | 108 | N | VAL | A | 7 | −26.096 | 82.516 | 5.947 | 1.00 | 45.41 | N |
| ATOM | 110 | CA | VAL | A | 7 | −24.852 | 82.374 | 5.197 | 1.00 | 52.46 | C |
| ATOM | 112 | CB | VAL | A | 7 | −24.078 | 83.738 | 5.072 | 1.00 | 57.08 | C |
| ATOM | 114 | CG1 | VAL | A | 7 | −22.667 | 83.569 | 4.503 | 1.00 | 60.43 | C |
| ATOM | 118 | CG2 | VAL | A | 7 | −24.843 | 84.697 | 4.154 | 1.00 | 54.32 | C |
| ATOM | 122 | C | VAL | A | 7 | −24.052 | 81.222 | 5.799 | 1.00 | 38.00 | C |
| ATOM | 123 | O | VAL | A | 7 | −23.381 | 80.509 | 5.094 | 1.00 | 37.86 | O |
| ATOM | 124 | N | GLN | A | 8 | −24.212 | 81.006 | 7.095 | 1.00 | 47.21 | N |
| ATOM | 126 | CA | GLN | A | 8 | −23.433 | 80.053 | 7.881 | 1.00 | 48.02 | C |
| ATOM | 128 | CB | GLN | A | 8 | −22.923 | 80.725 | 9.152 | 1.00 | 52.51 | C |
| ATOM | 131 | CG | GLN | A | 8 | −21.688 | 80.108 | 9.783 | 1.00 | 59.84 | C |
| ATOM | 134 | CD | GLN | A | 8 | −21.703 | 80.308 | 11.284 | 1.00 | 69.57 | C |
| ATOM | 135 | OE1 | GLN | A | 8 | −22.046 | 81.394 | 11.747 | 1.00 | 89.27 | O |
| ATOM | 136 | NE2 | GLN | A | 8 | −21.401 | 79.259 | 12.044 | 1.00 | 82.48 | N |
| ATOM | 139 | C | GLN | A | 8 | −24.362 | 78.937 | 8.310 | 1.00 | 56.36 | C |
| ATOM | 140 | O | GLN | A | 8 | −24.863 | 78.908 | 9.448 | 1.00 | 56.93 | O |
| ATOM | 141 | N | GLY | A | 9 | −24.611 | 78.038 | 7.361 | 1.00 | 61.89 | N |
| ATOM | 143 | CA | GLY | A | 9 | −25.725 | 77.114 | 7.450 | 1.00 | 52.82 | C |
| ATOM | 146 | C | GLY | A | 9 | −25.256 | 75.970 | 8.329 | 1.00 | 51.63 | C |
| ATOM | 147 | O | GLY | A | 9 | −26.090 | 75.176 | 8.749 | 1.00 | 43.12 | O |
| ATOM | 148 | N | SER | A | 10 | −23.948 | 75.862 | 8.588 | 1.00 | 48.31 | N |
| ATOM | 150 | CA | SER | A | 10 | −23.447 | 74.957 | 9.629 | 1.00 | 48.60 | C |
| ATOM | 152 | CB | SER | A | 10 | −22.116 | 75.451 | 10.167 | 1.00 | 49.40 | C |
| ATOM | 155 | OG | SER | A | 10 | −21.255 | 75.762 | 9.096 | 1.00 | 69.91 | O |
| ATOM | 157 | C | SER | A | 10 | −24.416 | 74.911 | 10.815 | 1.00 | 48.26 | C |
| ATOM | 158 | O | SER | A | 10 | −24.817 | 73.864 | 11.277 | 1.00 | 33.04 | O |
| ATOM | 159 | N | ILE | A | 11 | −24.806 | 76.063 | 11.327 | 1.00 | 48.26 | N |
| ATOM | 161 | CA | ILE | A | 11 | −25.654 | 76.083 | 12.495 | 1.00 | 47.87 | C |
| ATOM | 163 | CB | ILE | A | 11 | −26.009 | 77.561 | 12.723 | 1.00 | 54.36 | C |
| ATOM | 165 | CG1 | ILE | A | 11 | −24.760 | 78.355 | 13.094 | 1.00 | 48.04 | C |
| ATOM | 168 | CD1 | ILE | A | 11 | −24.968 | 79.837 | 12.910 | 1.00 | 67.07 | C |
| ATOM | 172 | CG2 | ILE | A | 11 | −27.010 | 77.727 | 13.828 | 1.00 | 66.02 | C |
| ATOM | 176 | C | ILE | A | 11 | −26.906 | 75.203 | 12.282 | 1.00 | 45.67 | C |
| ATOM | 177 | O | ILE | A | 11 | −27.364 | 74.484 | 13.172 | 1.00 | 42.31 | O |
| ATOM | 178 | N | LEU | A | 12 | −27.499 | 75.251 | 11.099 | 1.00 | 42.85 | N |
| ATOM | 180 | CA | LEU | A | 12 | −28.714 | 74.473 | 10.900 | 1.00 | 46.77 | C |
| ATOM | 182 | CB | LEU | A | 12 | −29.432 | 74.824 | 9.586 | 1.00 | 42.85 | C |
| ATOM | 185 | CG | LEU | A | 12 | −30.452 | 75.921 | 9.871 | 1.00 | 52.28 | C |
| ATOM | 187 | CD1 | LEU | A | 12 | −30.870 | 76.658 | 8.622 | 1.00 | 62.44 | C |
| ATOM | 191 | CD2 | LEU | A | 12 | −31.660 | 75.338 | 10.614 | 1.00 | 57.23 | C |
| ATOM | 195 | C | LEU | A | 12 | −28.381 | 72.984 | 10.995 | 1.00 | 45.53 | C |
| ATOM | 196 | O | LEU | A | 12 | −29.153 | 72.180 | 11.524 | 1.00 | 40.61 | O |
| ATOM | 197 | N | LYS | A | 13 | −27.212 | 72.630 | 10.482 | 1.00 | 39.67 | N |
| ATOM | 199 | CA | LYS | A | 13 | −26.816 | 71.241 | 10.451 | 1.00 | 36.73 | C |
| ATOM | 201 | CB | LYS | A | 13 | −25.464 | 71.065 | 9.755 | 1.00 | 37.27 | C |
| ATOM | 204 | CG | LYS | A | 13 | −25.247 | 71.743 | 8.397 | 1.00 | 40.13 | C |
| ATOM | 207 | CD | LYS | A | 13 | −25.194 | 70.737 | 7.262 | 1.00 | 54.88 | C |
| ATOM | 210 | CE | LYS | A | 13 | −23.981 | 69.825 | 7.259 | 1.00 | 50.68 | C |
| ATOM | 213 | NZ | LYS | A | 13 | −22.788 | 70.728 | 7.189 | 1.00 | 41.22 | N |
| ATOM | 217 | C | LYS | A | 13 | −26.681 | 70.755 | 11.884 | 1.00 | 41.67 | C |
| ATOM | 218 | O | LYS | A | 13 | −27.293 | 69.750 | 12.253 | 1.00 | 54.10 | O |
| ATOM | 219 | N | LYS | A | 14 | −25.874 | 71.464 | 12.667 | 1.00 | 33.60 | N |
| ATOM | 221 | CA | LYS | A | 14 | −25.676 | 71.222 | 14.092 | 1.00 | 31.66 | C |
| ATOM | 223 | CB | LYS | A | 14 | −24.814 | 72.312 | 14.715 | 1.00 | 33.08 | C |
| ATOM | 226 | CG | LYS | A | 14 | −23.536 | 72.511 | 13.892 | 1.00 | 34.44 | C |
| ATOM | 229 | CD | LYS | A | 14 | −22.299 | 72.922 | 14.643 | 1.00 | 34.15 | C |
| ATOM | 232 | CE | LYS | A | 14 | −21.177 | 73.202 | 13.641 | 1.00 | 43.33 | C |
| ATOM | 235 | NZ | LYS | A | 14 | −20.056 | 73.933 | 14.292 | 1.00 | 56.53 | N |
| ATOM | 239 | C | LYS | A | 14 | −26.934 | 71.094 | 14.899 | 1.00 | 33.86 | C |
| ATOM | 240 | O | LYS | A | 14 | −27.052 | 70.194 | 15.730 | 1.00 | 46.50 | O |
| ATOM | 241 | N | VAL | A | 15 | −27.894 | 71.967 | 14.659 | 1.00 | 39.86 | N |
| ATOM | 243 | CA | VAL | A | 15 | −29.168 | 71.874 | 25.388 | 1.00 | 38.86 | C |
| ATOM | 245 | CB | VAL | A | 15 | −30.111 | 73.024 | 14.986 | 1.00 | 37.52 | C |
| ATOM | 247 | CG1 | VAL | A | 15 | −31.513 | 72.754 | 15.472 | 1.00 | 42.92 | C |
| ATOM | 251 | CG2 | VAL | A | 15 | −29.581 | 74.377 | 15.506 | 1.00 | 44.19 | C |
| ATOM | 255 | C | VAL | A | 15 | −29.839 | 70.545 | 15.069 | 1.00 | 35.19 | C |
| ATOM | 256 | O | VAL | A | 15 | −30.349 | 69.833 | 15.945 | 1.00 | 39.61 | O |
| ATOM | 257 | N | LEU | A | 16 | −29.860 | 70.183 | 13.795 | 1.00 | 33.99 | N |
| ATOM | 259 | CA | LEU | A | 16 | −30.637 | 69.009 | 13.463 | 1.00 | 34.64 | C |
| ATOM | 261 | CB | LEU | A | 16 | −30.912 | 68.902 | 11.969 | 1.00 | 26.05 | C |
| ATOM | 264 | CG | LEU | A | 16 | −32.254 | 69.465 | 11.466 | 1.00 | 45.86 | C |
| ATOM | 266 | CD1 | LEU | A | 16 | −33.509 | 68.708 | 11.920 | 1.00 | 50.90 | C |
| ATOM | 270 | CD2 | LEU | A | 16 | −32.401 | 70.897 | 11.884 | 1.00 | 47.59 | C |
| ATOM | 274 | C | LEU | A | 16 | −29.936 | 67.777 | 14.041 | 1.00 | 35.09 | C |
| ATOM | 275 | O | LEU | A | 16 | −30.577 | 66.875 | 14.509 | 1.00 | 38.28 | O |
| ATOM | 276 | N | GLU | A | 17 | −28.615 | 67.753 | 14.098 | 1.00 | 33.65 | N |
| ATOM | 278 | CA | GLU | A | 17 | −27.953 | 66.626 | 14.712 | 1.00 | 34.53 | C |
| ATOM | 280 | CB | GLU | A | 17 | −26.436 | 66.680 | 14.487 | 1.00 | 39.09 | C |
| ATOM | 283 | CG | GLU | A | 17 | −25.979 | 66.580 | 13.040 | 1.00 | 60.19 | C |
| ATOM | 286 | CD | GLU | A | 17 | −26.190 | 65.202 | 12.458 | 1.00 | 65.85 | C |
| ATOM | 287 | OE1 | GLU | A | 17 | −25.341 | 64.364 | 12.820 | 1.00 | 72.09 | O |
| ATOM | 288 | OE2 | GLU | A | 17 | −27.185 | 64.988 | 11.699 | 1.00 | 63.94 | O |
| ATOM | 289 | C | GLU | A | 17 | −28.181 | 66.622 | 16.206 | 1.00 | 33.10 | C |
| ATOM | 290 | O | GLU | A | 17 | −27.998 | 65.605 | 16.844 | 1.00 | 44.36 | O |
| ATOM | 291 | N | ALA | A | 18 | −28.440 | 67.770 | 16.797 | 1.00 | 32.65 | N |
| ATOM | 293 | CA | ALA | A | 18 | −28.482 | 67.874 | 18.239 | 1.00 | 41.03 | C |
| ATOM | 295 | CB | ALA | A | 18 | −28.266 | 69.351 | 18.675 | 1.00 | 43.40 | C |
| ATOM | 299 | C | ALA | A | 18 | −29.846 | 67.357 | 18.693 | 1.00 | 44.40 | C |
| ATOM | 300 | O | ALA | A | 18 | −30.088 | 67.120 | 19.891 | 1.00 | 54.06 | O |
| ATOM | 301 | N | LEU | A | 19 | −30.707 | 67.150 | 17.702 | 1.00 | 46.12 | N |
| ATOM | 303 | CA | LEU | A | 19 | −32.138 | 66.919 | 17.895 | 1.00 | 45.76 | C |
| ATOM | 305 | CB | LEU | A | 19 | −32.883 | 68.000 | 17.112 | 1.00 | 51.41 | C |
| ATOM | 308 | CG | LEU | A | 19 | −32.958 | 69.410 | 17.683 | 1.00 | 49.96 | C |
| ATOM | 310 | CD1 | LEU | A | 19 | −34.017 | 70.154 | 16.892 | 1.00 | 33.74 | C |
| ATOM | 314 | CD2 | LEU | A | 19 | −33.298 | 69.370 | 19.162 | 1.00 | 61.86 | C |
| ATOM | 318 | C | LEU | A | 19 | −32.623 | 65.549 | 17.381 | 1.00 | 37.43 | C |
| ATOM | 319 | O | LEU | A | 19 | −33.475 | 64.912 | 17.994 | 1.00 | 41.80 | O |
| ATOM | 320 | N | LYS | A | 20 | −32.141 | 65.154 | 16.208 | 1.00 | 34.17 | N |
| ATOM | 322 | CA | LYS | A | 20 | −32.668 | 64.026 | 15.441 | 1.00 | 40.52 | C |
| ATOM | 324 | CB | LYS | A | 20 | −31.868 | 63.815 | 14.135 | 1.00 | 28.20 | C |
| ATOM | 327 | CG | LYS | A | 20 | −30.509 | 63.189 | 14.387 | 1.00 | 53.41 | C |
| ATOM | 330 | CD | LYS | A | 20 | −29.940 | 62.463 | 13.188 | 1.00 | 58.85 | C |
| ATOM | 333 | CE | LYS | A | 20 | −28.736 | 61.636 | 13.601 | 1.00 | 53.06 | C |
| ATOM | 336 | NZ | LYS | A | 20 | −27.556 | 61.954 | 12.730 | 1.00 | 77.74 | N |
| ATOM | 340 | C | LYS | A | 20 | −32.752 | 62.718 | 16.251 | 1.00 | 33.59 | C |
| ATOM | 341 | O | LYS | A | 20 | −33.703 | 61.963 | 16.121 | 1.00 | 49.68 | O |
| ATOM | 342 | N | ASP | A | 21 | −31.798 | 62.482 | 17.130 | 1.00 | 38.54 | N |
| ATOM | 344 | CA | ASP | A | 21 | −31.781 | 61.277 | 17.954 | 1.00 | 43.08 | C |
| ATOM | 346 | CB | ASP | A | 21 | −30.348 | 60.928 | 18.410 | 1.00 | 45.74 | C |
| ATOM | 349 | CG | ASP | A | 21 | −29.435 | 60.628 | 17.247 | 1.00 | 44.16 | C |
| ATOM | 350 | OD1 | ASP | A | 21 | −29.970 | 60.090 | 16.256 | 1.00 | 50.69 | O |
| ATOM | 351 | OD2 | ASP | A | 21 | −28.224 | 60.950 | 17.188 | 1.00 | 72.68 | O |
| ATOM | 352 | C | ASP | A | 21 | −32.685 | 61.353 | 19.180 | 1.00 | 41.64 | C |
| ATOM | 353 | O | ASP | A | 21 | −33.183 | 60.328 | 19.607 | 1.00 | 45.31 | O |
| ATOM | 354 | N | LEU | A | 22 | −32.832 | 62.523 | 19.793 | 1.00 | 41.93 | N |
| ATOM | 356 | CA | LEU | A | 22 | −33.769 | 62.665 | 20.906 | 1.00 | 39.44 | C |
| ATOM | 358 | CB | LEU | A | 22 | −33.539 | 63.953 | 21.682 | 1.00 | 33.74 | C |
| ATOM | 361 | CG | LEU | A | 22 | −34.295 | 63.829 | 23.005 | 1.00 | 30.64 | C |
| ATOM | 363 | CD1 | LEU | A | 22 | −33.629 | 62.719 | 23.828 | 1.00 | 56.41 | C |
| ATOM | 367 | CD2 | LEU | A | 22 | −34.258 | 65.068 | 23.848 | 1.00 | 51.72 | C |
| ATOM | 371 | C | LEU | A | 22 | −35.230 | 62.700 | 20.486 | 1.00 | 42.63 | C |
| ATOM | 372 | O | LEU | A | 22 | −36.077 | 62.171 | 21.182 | 1.00 | 50.26 | O |
| ATOM | 373 | N | ILE | A | 23 | −35.542 | 63.416 | 19.413 | 1.00 | 44.40 | N |
| ATOM | 375 | CA | ILE | A | 23 | −36.908 | 63.483 | 18.930 | 1.00 | 43.45 | C |
| ATOM | 377 | CB | ILE | A | 23 | −37.498 | 64.851 | 19.272 | 1.00 | 47.78 | C |
| ATOM | 379 | CG1 | ILE | A | 23 | −36.896 | 65.886 | 18.365 | 1.00 | 43.39 | C |
| ATOM | 382 | CD1 | ILE | A | 23 | −37.258 | 67.249 | 18.857 | 1.00 | 66.17 | C |
| ATOM | 386 | CG2 | ILE | A | 23 | −37.206 | 65.286 | 20.733 | 1.00 | 38.24 | C |
| ATOM | 390 | C | ILE | A | 23 | −36.968 | 63.271 | 17.429 | 1.00 | 47.67 | C |
| ATOM | 391 | O | ILE | A | 23 | −35.992 | 63.512 | 16.716 | 1.00 | 56.01 | O |
| ATOM | 392 | N | ASN | A | 24 | −38.103 | 62.809 | 16.929 | 1.00 | 53.21 | N |
| ATOM | 394 | CA | ASN | A | 24 | −38.173 | 62.402 | 15.522 | 1.00 | 62.63 | C |
| ATOM | 396 | CB | ASN | A | 24 | −38.547 | 60.922 | 15.358 | 1.00 | 66.33 | C |
| ATOM | 399 | CG | ASN | A | 24 | −38.609 | 60.183 | 16.684 | 1.00 | 88.18 | C |
| ATOM | 400 | OD1 | ASN | A | 24 | −37.790 | 59.289 | 16.958 | 1.00 | 94.98 | O |
| ATOM | 401 | ND2 | ASN | A | 24 | −39.570 | 60.577 | 17.532 | 1.00 | 99.37 | N |
| ATOM | 404 | C | ASN | A | 24 | −39.153 | 63.232 | 14.720 | 1.00 | 61.52 | C |
| ATOM | 405 | O | ASN | A | 24 | −38.984 | 63.399 | 13.519 | 1.00 | 66.81 | O |
| ATOM | 406 | N | GLU | A | 25 | −40.205 | 63.707 | 15.371 | 1.00 | 61.26 | N |
| ATOM | 408 | CA | GLU | A | 25 | −41.041 | 64.728 | 14.788 | 1.00 | 58.53 | C |
| ATOM | 410 | CB | GLU | A | 25 | −42.438 | 64.195 | 14.405 | 1.00 | 65.83 | C |
| ATOM | 413 | CG | GLU | A | 25 | −42.806 | 62.767 | 14.781 | 1.00 | 75.66 | C |
| ATOM | 416 | CD | GLU | A | 25 | −43.896 | 62.714 | 15.854 | 1.00 | 93.47 | C |
| ATOM | 417 | OE1 | GLU | A | 25 | −45.015 | 62.245 | 15.551 | 1.00 | 104.27 | O |
| ATOM | 418 | OE2 | GLU | A | 25 | −43.656 | 63.126 | 17.016 | 1.00 | 92.84 | O |
| ATOM | 419 | C | GLU | A | 25 | −41.120 | 65.924 | 15.722 | 1.00 | 53.87 | C |
| ATOM | 420 | O | GLU | A | 25 | −40.984 | 65.810 | 16.929 | 1.00 | 53.21 | O |
| ATOM | 421 | N | ALA | A | 26 | −41.308 | 67.103 | 15.151 | 1.00 | 51.19 | N |
| ATOM | 423 | CA | ALA | A | 26 | −41.557 | 68.285 | 15.959 | 1.00 | 48.21 | C |
| ATOM | 425 | CB | ALA | A | 26 | −40.321 | 68.728 | 16.707 | 1.00 | 37.99 | C |
| ATOM | 429 | C | ALA | A | 26 | −42.078 | 69.438 | 15.111 | 1.00 | 53.72 | C |
| ATOM | 430 | O | ALA | A | 26 | −41.882 | 69.529 | 13.884 | 1.00 | 48.65 | O |
| ATOM | 431 | N | CYS | A | 27 | −42.776 | 70.327 | 15.805 | 1.00 | 51.69 | N |
| ATOM | 433 | CA | CYS | A | 27 | −43.401 | 71.411 | 15.114 | 1.00 | 49.92 | C |
| ATOM | 435 | CB | CYS | A | 27 | −44.838 | 71.584 | 15.573 | 1.00 | 52.17 | C |
| ATOM | 438 | SG | CYS | A | 27 | −45.475 | 73.220 | 15.146 | 1.00 | 59.20 | S |
| ATOM | 439 | C | CYS | A | 27 | −42.547 | 72.673 | 15.252 | 1.00 | 46.45 | C |
| ATOM | 440 | O | CYS | A | 27 | −42.112 | 73.081 | 16.339 | 1.00 | 40.25 | O |
| ATOM | 441 | N | TRP | A | 28 | −42.234 | 73.221 | 14.085 | 1.00 | 41.77 | N |
| ATOM | 443 | CA | TRP | A | 28 | −41.416 | 74.401 | 14.001 | 1.00 | 40.64 | C |
| ATOM | 445 | CB | TRP | A | 28 | −40.523 | 74.291 | 12.768 | 1.00 | 47.79 | C |
| ATOM | 448 | CG | TRP | A | 28 | −39.381 | 73.287 | 12.872 | 1.00 | 37.76 | C |
| ATOM | 449 | CD1 | TRP | A | 28 | −39.455 | 71.923 | 12.943 | 1.00 | 53.70 | C |
| ATOM | 451 | NE1 | TRP | A | 28 | −38.196 | 71.370 | 12.979 | 1.00 | 38.70 | N |
| ATOM | 453 | CE2 | TRP | A | 28 | −37.284 | 72.382 | 12.902 | 1.00 | 49.57 | C |
| ATOM | 454 | CD2 | TRP | A | 28 | −37.996 | 73.595 | 12.813 | 1.00 | 52.87 | C |
| ATOM | 455 | CE3 | TRP | A | 28 | −37.276 | 74.790 | 12.712 | 1.00 | 54.09 | C |
| ATOM | 457 | CZ3 | TRP | A | 28 | −35.928 | 74.729 | 12.694 | 1.00 | 57.34 | C |
| ATOM | 459 | CH2 | TRP | A | 28 | −35.258 | 73.509 | 12.700 | 1.00 | 37.65 | C |
| ATOM | 461 | CZ2 | TRP | A | 28 | −35.915 | 72.329 | 12.827 | 1.00 | 42.89 | C |
| ATOM | 463 | C | TRP | A | 28 | −42.424 | 75.533 | 13.861 | 1.00 | 46.08 | C |
| ATOM | 464 | O | TRP | A | 28 | −43.123 | 75.642 | 12.843 | 1.00 | 43.28 | O |
| ATOM | 465 | N | ASP | A | 29 | −42.557 | 76.301 | 14.945 | 1.00 | 50.13 | N |
| ATOM | 467 | CA | ASP | A | 29 | −43.291 | 77.566 | 14.981 | 1.00 | 40.23 | C |
| ATOM | 469 | CB | ASP | A | 29 | −43.675 | 77.856 | 16.414 | 1.00 | 36.40 | C |
| ATOM | 472 | CG | ASP | A | 29 | −44.609 | 76.815 | 16.963 | 1.00 | 40.14 | C |
| ATOM | 473 | OD1 | ASP | A | 29 | −45.666 | 76.598 | 16.337 | 1.00 | 65.62 | O |
| ATOM | 474 | OD2 | ASP | A | 29 | −44.354 | 76.145 | 17.980 | 1.00 | 54.44 | O |
| ATOM | 475 | C | ASP | A | 29 | −42.490 | 78.739 | 14.432 | 1.00 | 44.84 | C |
| ATOM | 476 | O | ASP | A | 29 | −41.548 | 79.230 | 15.052 | 1.00 | 47.82 | O |
| ATOM | 477 | N | ILE | A | 30 | −42.885 | 79.192 | 13.252 | 1.00 | 48.46 | N |
| ATOM | 479 | CA | ILE | A | 30 | −42.304 | 80.381 | 12.667 | 1.00 | 49.16 | C |
| ATOM | 481 | CB | ILE | A | 30 | −42.103 | 80.154 | 11.183 | 1.00 | 49.96 | C |
| ATOM | 483 | CG1 | ILE | A | 30 | −41.076 | 79.035 | 11.023 | 1.00 | 41.45 | C |
| ATOM | 486 | CD1 | ILE | A | 30 | −41.700 | 77.807 | 10.532 | 1.00 | 59.88 | C |
| ATOM | 490 | CG2 | ILE | A | 30 | −41.618 | 81.456 | 10.526 | 1.00 | 53.59 | C |
| ATOM | 494 | C | ILE | A | 30 | −43.171 | 81.607 | 12.875 | 1.00 | 51.63 | C |
| ATOM | 495 | O | ILE | A | 30 | −44.363 | 81.599 | 12.578 | 1.00 | 41.80 | O |
| ATOM | 496 | N | SER | A | 31 | −42.550 | 82.671 | 13.371 | 1.00 | 53.36 | N |
| ATOM | 498 | CA | SER | A | 31 | −43.171 | 83.990 | 13.393 | 1.00 | 47.79 | C |
| ATOM | 500 | CB | SER | A | 31 | −43.632 | 84.276 | 14.814 | 1.00 | 55.09 | C |
| ATOM | 503 | OG | SER | A | 31 | −42.529 | 84.650 | 15.640 | 1.00 | 48.03 | O |
| ATOM | 505 | C | SER | A | 31 | −42.116 | 85.034 | 13.079 | 1.00 | 48.79 | C |
| ATOM | 506 | O | SER | A | 31 | −40.932 | 84.742 | 12.934 | 1.00 | 54.75 | O |
| ATOM | 507 | N | SER | A | 32 | −42.524 | 86.287 | 13.091 | 1.00 | 50.62 | N |
| ATOM | 509 | CA | SER | A | 32 | −41.638 | 87.355 | 12.694 | 1.00 | 48.23 | C |
| ATOM | 511 | CB | SER | A | 32 | −42.506 | 88.567 | 12.483 | 1.00 | 51.07 | C |
| ATOM | 514 | OG | SER | A | 32 | −43.759 | 88.084 | 12.046 | 1.00 | 76.65 | O |
| ATOM | 516 | C | SER | A | 32 | −40.573 | 87.633 | 13.736 | 1.00 | 46.24 | C |
| ATOM | 517 | O | SER | A | 32 | −39.559 | 88.263 | 13.459 | 1.00 | 54.83 | O |
| ATOM | 518 | N | SER | A | 33 | −40.763 | 87.156 | 14.950 | 1.00 | 43.42 | N |
| ATOM | 520 | CA | SER | A | 33 | −39.665 | 87.256 | 15.905 | 1.00 | 46.24 | C |
| ATOM | 522 | CB | SER | A | 33 | −40.194 | 87.207 | 17.340 | 1.00 | 39.44 | C |
| ATOM | 525 | OG | SER | A | 33 | −40.553 | 85.880 | 17.650 | 1.00 | 53.77 | O |
| ATOM | 527 | C | SER | A | 33 | −38.611 | 86.157 | 15.697 | 1.00 | 47.24 | C |
| ATOM | 528 | O | SER | A | 33 | −37.534 | 86.242 | 16.297 | 1.00 | 42.57 | O |
| ATOM | 529 | N | GLY | A | 34 | −38.957 | 85.138 | 14.893 | 1.00 | 43.90 | N |
| ATOM | 531 | CA | GLY | A | 34 | −38.078 | 84.048 | 14.513 | 1.00 | 42.54 | C |
| ATOM | 534 | C | GLY | A | 34 | −38.640 | 82.628 | 14.576 | 1.00 | 41.98 | C |
| ATOM | 535 | O | GLY | A | 34 | −39.776 | 82.361 | 14.181 | 1.00 | 50.11 | O |
| ATOM | 536 | N | VAL | A | 35 | −37.800 | 81.686 | 15.014 | 1.00 | 45.72 | N |
| ATOM | 538 | CA | VAL | A | 35 | −38.079 | 80.245 | 14.934 | 1.00 | 40.70 | C |
| ATOM | 540 | CB | VAL | A | 35 | −37.083 | 79.436 | 14.060 | 1.00 | 36.79 | C |
| ATOM | 542 | CG1 | VAL | A | 35 | −37.384 | 77.925 | 14.185 | 1.00 | 39.48 | C |
| ATOM | 546 | CG2 | VAL | A | 35 | −37.251 | 79.780 | 12.553 | 1.00 | 43.00 | C |
| ATOM | 550 | C | VAL | A | 35 | −38.103 | 79.642 | 16.331 | 1.00 | 42.70 | C |
| ATOM | 551 | O | VAL | A | 35 | −37.261 | 79.926 | 17.174 | 1.00 | 46.59 | O |
| ATOM | 552 | N | ASN | A | 36 | −39.127 | 78.856 | 16.608 | 1.00 | 46.78 | N |
| ATOM | 554 | CA | ASN | A | 36 | −39.268 | 78.295 | 17.934 | 1.00 | 50.53 | C |
| ATOM | 556 | CB | ASN | A | 36 | −40.271 | 79.108 | 18.731 | 1.00 | 56.15 | C |
| ATOM | 559 | CG | ASN | A | 36 | −39.619 | 80.144 | 19.620 | 1.00 | 65.44 | C |
| ATOM | 560 | OD1 | ASN | A | 36 | −39.353 | 81.281 | 19.205 | 1.00 | 76.13 | O |
| ATOM | 561 | ND2 | ASN | A | 36 | −39.421 | 79.772 | 20.885 | 1.00 | 83.68 | N |
| ATOM | 564 | C | ASN | A | 36 | −39.705 | 76.836 | 17.841 | 1.00 | 53.23 | C |
| ATOM | 565 | O | ASN | A | 36 | −40.492 | 76.447 | 16.966 | 1.00 | 49.85 | O |
| ATOM | 566 | N | LEU | A | 37 | −39.105 | 76.019 | 18.701 | 1.00 | 52.66 | N |
| ATOM | 568 | CA | LEU | A | 37 | −39.382 | 74.594 | 18.705 | 1.00 | 49.12 | C |
| ATOM | 570 | CB | LEU | A | 37 | −38.336 | 73.860 | 17.893 | 1.00 | 49.14 | C |
| ATOM | 573 | CG | LEU | A | 37 | −38.787 | 72.452 | 17.520 | 1.00 | 48.57 | C |
| ATOM | 575 | CD1 | LEU | A | 37 | −38.338 | 72.064 | 16.114 | 1.00 | 52.04 | C |
| ATOM | 579 | CD2 | LEU | A | 37 | −38.242 | 71.485 | 18.546 | 1.00 | 46.42 | C |
| ATOM | 583 | C | LEU | A | 37 | −39.429 | 74.036 | 20.115 | 1.00 | 44.85 | C |
| ATOM | 584 | O | LEU | A | 37 | −38.531 | 74.278 | 20.901 | 1.00 | 45.86 | O |
| ATOM | 585 | N | GLN | A | 38 | −40.520 | 73.352 | 20.448 | 1.00 | 47.86 | N |
| ATOM | 587 | CA | GLN | A | 38 | −40.663 | 72.676 | 21.730 | 1.00 | 44.25 | C |
| ATOM | 589 | CB | GLN | A | 38 | −41.565 | 73.491 | 22.644 | 1.00 | 42.24 | C |
| ATOM | 592 | CG | GLN | A | 38 | −41.648 | 72.876 | 24.043 | 1.00 | 59.76 | C |
| ATOM | 595 | CD | GLN | A | 38 | −42.818 | 73.388 | 24.879 | 1.00 | 50.18 | C |
| ATOM | 596 | OE1 | GLN | A | 38 | −43.965 | 72.909 | 24.762 | 1.00 | 48.71 | O |
| ATOM | 597 | NE2 | GLN | A | 38 | −42.509 | 74.313 | 25.768 | 1.00 | 54.10 | N |
| ATOM | 600 | C | GLN | A | 38 | −41.244 | 71.281 | 21.502 | 1.00 | 41.54 | C |
| ATOM | 601 | O | GLN | A | 38 | −42.272 | 71.101 | 20.872 | 1.00 | 37.38 | O |
| ATOM | 602 | N | SER | A | 39 | −40.503 | 70.266 | 21.886 | 1.00 | 42.85 | N |
| ATOM | 604 | CA | SER | A | 39 | −40.955 | 68.902 | 21.656 | 1.00 | 42.03 | C |
| ATOM | 606 | CB | SER | A | 39 | −40.531 | 68.339 | 20.308 | 1.00 | 44.30 | C |
| ATOM | 609 | OG | SER | A | 39 | −41.026 | 67.029 | 20.087 | 1.00 | 45.03 | O |
| ATOM | 611 | C | SER | A | 39 | −40.445 | 68.020 | 22.783 | 1.00 | 47.46 | C |
| ATOM | 612 | O | SER | A | 39 | −39.535 | 68.361 | 23.564 | 1.00 | 48.30 | O |
| ATOM | 613 | N | MET | A | 40 | −41.166 | 66.927 | 22.956 | 1.00 | 44.24 | N |
| ATOM | 615 | CA | MET | A | 40 | −40.808 | 66.002 | 23.996 | 1.00 | 45.69 | C |
| ATOM | 617 | CB | MET | A | 40 | −41.985 | 65.838 | 24.942 | 1.00 | 47.39 | C |
| ATOM | 620 | CG | MET | A | 40 | −41.991 | 66.735 | 26.155 | 1.00 | 53.62 | C |
| ATOM | 623 | SD | MET | A | 40 | −43.263 | 66.178 | 27.388 | 1.00 | 59.83 | S |
| ATOM | 624 | CE | MET | A | 40 | −44.600 | 65.752 | 26.388 | 1.00 | 64.55 | C |
| ATOM | 628 | C | MET | A | 40 | −40.482 | 64.700 | 23.269 | 1.00 | 46.62 | C |
| ATOM | 629 | O | MET | A | 40 | −40.911 | 64.428 | 22.137 | 1.00 | 51.14 | O |
| ATOM | 630 | N | ASP | A | 41 | −39.682 | 63.892 | 23.930 | 1.00 | 40.85 | N |
| ATOM | 632 | CA | ASP | A | 41 | −39.313 | 62.619 | 23.356 | 1.00 | 47.21 | C |
| ATOM | 634 | CB | ASP | A | 41 | −37.954 | 62.177 | 23.923 | 1.00 | 49.91 | C |
| ATOM | 637 | CG | ASP | A | 41 | −38.017 | 61.829 | 25.401 | 1.00 | 54.94 | C |
| ATOM | 638 | OD1 | ASP | A | 41 | −38.043 | 62.729 | 26.281 | 1.00 | 62.51 | O |
| ATOM | 639 | OD2 | ASP | A | 41 | −38.071 | 60.644 | 25.777 | 1.00 | 46.93 | O |
| ATOM | 640 | C | ASP | A | 41 | −40.417 | 61.668 | 23.787 | 1.00 | 47.30 | C |
| ATOM | 641 | O | ASP | A | 41 | −41.150 | 61.961 | 24.753 | 1.00 | 40.12 | O |
| ATOM | 642 | N | SER | A | 42 | −40.470 | 60.519 | 23.116 | 1.00 | 43.35 | N |
| ATOM | 644 | CA | SER | A | 42 | −41.640 | 59.673 | 23.131 | 1.00 | 40.31 | C |
| ATOM | 646 | CB | SER | A | 42 | −41.494 | 58.577 | 22.096 | 1.00 | 38.53 | C |
| ATOM | 649 | OG | SER | A | 42 | −40.132 | 58.440 | 21.801 | 1.00 | 62.24 | O |
| ATOM | 651 | C | SER | A | 42 | −41.889 | 59.077 | 24.495 | 1.00 | 37.02 | C |
| ATOM | 652 | O | SER | A | 42 | −42.976 | 58.626 | 24.780 | 1.00 | 55.60 | O |
| ATOM | 653 | N | SER | A | 43 | −40.912 | 59.113 | 25.376 | 1.00 | 44.48 | N |
| ATOM | 655 | CA | SER | A | 43 | −41.145 | 58.759 | 26.774 | 1.00 | 48.79 | C |
| ATOM | 657 | CB | SER | A | 43 | −39.861 | 58.147 | 27.315 | 1.00 | 50.71 | C |
| ATOM | 660 | OG | SER | A | 43 | −38.929 | 59.191 | 27.574 | 1.00 | 65.49 | O |
| ATOM | 662 | C | SER | A | 43 | −41.537 | 59.923 | 27.702 | 1.00 | 48.77 | C |
| ATOM | 663 | O | SER | A | 43 | −41.639 | 59.752 | 28.917 | 1.00 | 49.67 | O |
| ATOM | 664 | N | HIS | A | 44 | −41.739 | 61.112 | 27.140 | 1.00 | 53.65 | N |
| ATOM | 666 | CA | HIS | A | 44 | −42.046 | 62.331 | 27.910 | 1.00 | 51.01 | C |
| ATOM | 668 | CB | HIS | A | 44 | −43.548 | 62.474 | 28.253 | 1.00 | 53.58 | C |
| ATOM | 671 | CG | HIS | A | 44 | −44.444 | 62.595 | 27.061 | 1.00 | 47.27 | C |
| ATOM | 672 | ND1 | HIS | A | 44 | −45.787 | 62.858 | 27.178 | 1.00 | 51.25 | N |
| ATOM | 674 | CE1 | HIS | A | 44 | −46.329 | 62.888 | 25.969 | 1.00 | 66.04 | C |
| ATOM | 676 | NE2 | HIS | A | 44 | −40.399 | 62.591 | 25.078 | 1.00 | 54.40 | N |
| ATOM | 678 | CD2 | HIS | A | 44 | −44.222 | 62.352 | 20.744 | 1.00 | 53.72 | C |
| ATOM | 680 | C | HIS | A | 44 | −41.246 | 62.454 | 29.199 | 1.00 | 43.65 | C |
| ATOM | 681 | O | HIS | A | 44 | −41.781 | 62.871 | 30.216 | 1.00 | 37.93 | O |
| ATOM | 682 | N | VAL | A | 45 | −39.947 | 62.192 | 29.121 | 1.00 | 52.71 | N |
| ATOM | 684 | CA | VAL | A | 45 | −39.041 | 62.350 | 30.266 | 1.00 | 52.37 | C |
| ATOM | 686 | CB | VAL | A | 45 | −38.024 | 61.180 | 30.289 | 1.00 | 58.48 | C |
| ATOM | 688 | CG1 | VAL | A | 45 | −36.958 | 61.370 | 31.358 | 1.00 | 55.43 | C |
| ATOM | 692 | CG2 | VAL | A | 45 | −38.730 | 59.827 | 30.395 | 1.00 | 53.40 | C |
| ATOM | 696 | C | VAL | A | 45 | −38.230 | 63.617 | 30.051 | 1.00 | 54.77 | C |
| ATOM | 697 | O | VAL | A | 45 | −37.795 | 64.295 | 31.001 | 1.00 | 49.48 | O |
| ATOM | 698 | N | SER | A | 46 | −37.965 | 63.872 | 28.770 | 1.00 | 51.22 | N |
| ATOM | 700 | CA | SER | A | 46 | −37.104 | 64.988 | 28.417 | 1.00 | 52.50 | C |
| ATOM | 702 | CB | SER | A | 46 | −35.831 | 64.491 | 27.718 | 1.00 | 53.24 | C |
| ATOM | 705 | OG | SER | A | 46 | −36.156 | 63.807 | 26.518 | 1.00 | 50.97 | O |
| ATOM | 707 | C | SER | A | 46 | −37.885 | 65.924 | 27.512 | 1.00 | 45.08 | C |
| ATOM | 708 | O | SER | A | 46 | −38.685 | 65.497 | 26.678 | 1.00 | 39.08 | O |
| ATOM | 709 | N | LEU | A | 47 | −37.620 | 67.213 | 27.665 | 1.00 | 45.05 | N |
| ATOM | 711 | CA | LEU | A | 47 | −38.147 | 68.202 | 26.730 | 1.00 | 48.82 | C |
| ATOM | 713 | CB | LEU | A | 47 | −39.179 | 69.080 | 27.431 | 1.00 | 52.32 | C |
| ATOM | 716 | CG | LEU | A | 47 | −39.819 | 70.174 | 26.586 | 1.00 | 52.61 | C |
| ATOM | 718 | CD1 | LEU | A | 47 | −41.184 | 70.441 | 27.186 | 1.00 | 50.81 | C |
| ATOM | 722 | CD2 | LEU | A | 47 | −38.916 | 71.413 | 26.657 | 1.00 | 36.50 | C |
| ATOM | 726 | C | LEU | A | 47 | −37.050 | 69.093 | 26.178 | 1.00 | 44.10 | C |
| ATOM | 727 | O | LEU | A | 47 | −36.220 | 69.602 | 26.943 | 1.00 | 38.80 | O |
| ATOM | 728 | N | VAL | A | 48 | −37.127 | 69.345 | 24.871 | 1.00 | 42.15 | N |
| ATOM | 730 | CA | VAL | A | 48 | −36.232 | 70.303 | 24.197 | 1.00 | 45.79 | C |
| ATOM | 732 | CB | VAL | A | 48 | −35.551 | 69.635 | 22.983 | 1.00 | 42.67 | C |
| ATOM | 734 | CG1 | VAL | A | 48 | −36.536 | 69.368 | 21.906 | 1.00 | 39.89 | C |
| ATOM | 738 | CG2 | VAL | A | 48 | −34.418 | 70.502 | 22.454 | 1.00 | 63.60 | C |
| ATOM | 742 | C | VAL | A | 48 | −36.878 | 71.641 | 23.778 | 1.00 | 42.16 | C |
| ATOM | 743 | O | VAL | A | 48 | −37.972 | 71.678 | 23.200 | 1.00 | 42.09 | O |
| ATOM | 744 | N | GLN | A | 49 | −36.225 | 72.763 | 24.063 | 1.00 | 43.84 | N |
| ATOM | 746 | CA | GLN | A | 49 | −36.669 | 74.005 | 23.416 | 1.00 | 47.29 | C |
| ATOM | 748 | CB | GLN | A | 49 | −37.275 | 74.980 | 24.409 | 1.00 | 49.18 | C |
| ATOM | 751 | CG | GLN | A | 49 | −37.460 | 76.393 | 23.875 | 1.00 | 57.75 | C |
| ATOM | 754 | CD | GLN | A | 49 | −37.052 | 77.394 | 24.924 | 1.00 | 86.40 | C |
| ATOM | 755 | OE1 | GLN | A | 49 | −37.533 | 77.333 | 26.078 | 1.00 | 80.75 | O |
| ATOM | 756 | NE2 | GLN | A | 49 | −36.112 | 78.274 | 24.560 | 1.00 | 98.68 | N |
| ATOM | 759 | C | GLN | A | 49 | −35.603 | 74.754 | 22.629 | 1.00 | 43.86 | C |
| ATOM | 760 | O | GLN | A | 49 | −34.581 | 75.142 | 23.200 | 1.00 | 43.58 | O |
| ATOM | 761 | N | LEU | A | 50 | −35.921 | 75.018 | 21.358 | 1.00 | 37.17 | N |
| ATOM | 763 | CA | LEU | A | 50 | −35.052 | 75.696 | 20.395 | 1.00 | 36.95 | C |
| ATOM | 765 | CB | LEU | A | 50 | −34.997 | 74.900 | 19.099 | 1.00 | 36.65 | C |
| ATOM | 768 | CG | LEU | A | 50 | −34.365 | 75.691 | 17.968 | 1.00 | 37.20 | C |
| ATOM | 770 | CD1 | LEU | A | 50 | −32.914 | 75.808 | 18.419 | 1.00 | 48.41 | C |
| ATOM | 774 | CD2 | LEU | A | 50 | −34.471 | 74.980 | 16.633 | 1.00 | 31.16 | C |
| ATOM | 778 | C | LEU | A | 50 | −35.616 | 77.070 | 20.047 | 1.00 | 40.74 | C |
| ATOM | 779 | O | LEU | A | 50 | −36.786 | 77.137 | 19.679 | 1.00 | 48.82 | O |
| ATOM | 780 | N | THR | A | 51 | −34.797 | 78.122 | 20.184 | 1.00 | 40.02 | N |
| ATOM | 782 | CA | THR | A | 51 | −35.096 | 79.502 | 19.806 | 1.00 | 44.94 | C |
| ATOM | 784 | CB | THR | A | 51 | −35.108 | 80.472 | 21.004 | 1.00 | 46.02 | C |
| ATOM | 786 | OG1 | THR | A | 51 | −34.368 | 79.917 | 22.090 | 1.00 | 67.16 | O |
| ATOM | 788 | CG2 | THR | A | 51 | −36.474 | 80.592 | 21.578 | 1.00 | 51.96 | C |
| ATOM | 792 | C | THR | A | 51 | −33.941 | 79.937 | 18.943 | 1.00 | 44.61 | C |
| ATOM | 793 | O | THR | A | 51 | −32.795 | 79.983 | 19.410 | 1.00 | 43.24 | O |
| ATOM | 794 | N | LEU | A | 52 | −34.278 | 80.179 | 17.681 | 1.00 | 42.27 | N |
| ATOM | 796 | CA | LEU | A | 52 | −33.486 | 80.991 | 16.791 | 1.00 | 39.78 | C |
| ATOM | 798 | CB | LEU | A | 52 | −33.172 | 80.241 | 15.501 | 1.00 | 35.12 | C |
| ATOM | 801 | CG | LEU | A | 52 | −32.744 | 78.794 | 15.618 | 1.00 | 36.86 | C |
| ATOM | 803 | CD1 | LEU | A | 52 | −33.021 | 78.093 | 14.331 | 1.00 | 20.48 | C |
| ATOM | 807 | CD2 | LEU | A | 52 | −31.298 | 78.716 | 15.958 | 1.00 | 35.52 | C |
| ATOM | 811 | C | LEU | A | 52 | −34.234 | 82.259 | 16.412 | 1.00 | 45.44 | C |
| ATOM | 812 | O | LEU | A | 52 | −35.170 | 82.241 | 15.587 | 1.00 | 40.88 | O |
| ATOM | 813 | N | ARG | A | 53 | −33.703 | 83.385 | 16.880 | 1.00 | 44.09 | N |
| ATOM | 815 | CA | ARG | A | 53 | −34.406 | 84.611 | 16.627 | 1.00 | 46.33 | C |
| ATOM | 817 | CB | ARG | A | 53 | −34.307 | 85.632 | 17.761 | 1.00 | 42.52 | C |
| ATOM | 820 | CG | ARG | A | 53 | −33.063 | 85.744 | 18.521 | 1.00 | 46.06 | C |
| ATOM | 823 | CD | ARG | A | 53 | −33.303 | 86.200 | 19.965 | 1.00 | 65.07 | C |
| ATOM | 826 | NE | ARG | A | 53 | −32.222 | 85.799 | 20.871 | 1.00 | 67.97 | N |
| ATOM | 828 | CZ | ARG | A | 53 | −30.944 | 86.142 | 20.719 | 1.00 | 81.29 | C |
| ATOM | 829 | NH1 | ARG | A | 53 | −30.512 | 86.887 | 19.682 | 1.00 | 69.60 | N |
| ATOM | 832 | NH2 | ARG | A | 53 | −30.088 | 85.686 | 21.623 | 1.00 | 83.75 | N |
| ATOM | 835 | C | ARG | A | 53 | −34.082 | 85.175 | 15.266 | 1.00 | 49.79 | C |
| ATOM | 836 | O | ARG | A | 53 | −33.023 | 84.859 | 14.693 | 1.00 | 52.93 | O |
| ATOM | 837 | N | SER | A | 54 | −35.032 | 85.970 | 14.763 | 1.00 | 44.61 | N |
| ATOM | 839 | CA | SER | A | 54 | −35.010 | 86.432 | 13.380 | 1.00 | 50.44 | C |
| ATOM | 841 | CB | SER | A | 54 | −36.281 | 87.202 | 13.002 | 1.00 | 44.24 | C |
| ATOM | 844 | OG | SER | A | 54 | −36.406 | 88.399 | 13.744 | 1.00 | 52.92 | O |
| ATOM | 846 | C | SER | A | 54 | −33.740 | 87.215 | 13.055 | 1.00 | 49.90 | C |
| ATOM | 847 | O | SER | A | 54 | −33.238 | 87.142 | 11.929 | 1.00 | 45.63 | O |
| ATOM | 848 | N | GLU | A | 55 | −33.193 | 87.919 | 14.042 | 1.00 | 52.53 | N |
| ATOM | 850 | CA | GLU | A | 55 | −32.115 | 88.854 | 13.751 | 1.00 | 54.53 | C |
| ATOM | 852 | CB | GLU | A | 55 | −31.883 | 89.821 | 14.901 | 1.00 | 54.69 | C |
| ATOM | 855 | CG | GLU | A | 55 | −33.178 | 90.454 | 15.353 | 1.00 | 72.07 | C |
| ATOM | 858 | CD | GLU | A | 55 | −33.672 | 89.877 | 16.657 | 1.00 | 83.48 | C |
| ATOM | 859 | OE1 | GLU | A | 55 | −34.491 | 88.930 | 16.621 | 1.00 | 77.61 | O |
| ATOM | 860 | OE2 | GLU | A | 55 | −33.235 | 90.398 | 17.706 | 1.00 | 96.83 | O |
| ATOM | 861 | C | GLU | A | 55 | −30.852 | 88.078 | 13.508 | 1.00 | 53.86 | C |
| ATOM | 862 | O | GLU | A | 55 | −29.915 | 88.611 | 12.944 | 1.00 | 52.78 | O |
| ATOM | 863 | N | GLY | A | 56 | −30.814 | 86.816 | 13.932 | 1.00 | 57.57 | N |
| ATOM | 865 | CA | GLY | A | 56 | −29.650 | 85.980 | 13.658 | 1.00 | 53.12 | C |
| ATOM | 868 | C | GLY | A | 56 | −29.488 | 85.576 | 12.201 | 1.00 | 45.71 | C |
| ATOM | 869 | O | GLY | A | 56 | −28.422 | 85.182 | 11.748 | 1.00 | 47.21 | O |
| ATOM | 870 | N | PHE | A | 57 | −30.589 | 85.595 | 11.474 | 1.00 | 41.74 | N |
| ATOM | 872 | CA | PHE | A | 57 | −30.569 | 85.114 | 10.112 | 1.00 | 47.63 | C |
| ATOM | 874 | CB | PHE | A | 57 | −31.974 | 84.736 | 9.631 | 1.00 | 43.51 | C |
| ATOM | 877 | CG | PHE | A | 57 | −32.446 | 83.461 | 10.235 | 1.00 | 42.49 | C |
| ATOM | 878 | CD1 | PHE | A | 57 | −32.934 | 83.465 | 11.540 | 1.00 | 43.15 | C |
| ATOM | 880 | CE1 | PHE | A | 57 | −33.299 | 82.318 | 12.153 | 1.00 | 27.38 | C |
| ATOM | 882 | CZ | PHE | A | 57 | −33.134 | 81.122 | 11.479 | 1.00 | 42.75 | C |
| ATOM | 884 | CE2 | PHE | A | 57 | −32.564 | 81.091 | 10.207 | 1.00 | 48.62 | C |
| ATOM | 886 | CD2 | PHE | A | 57 | −32.194 | 82.251 | 9.600 | 1.00 | 31.88 | C |
| ATOM | 888 | C | PHE | A | 57 | −30.026 | 86.246 | 9.298 | 1.00 | 49.57 | C |
| ATOM | 889 | O | PHE | A | 57 | −30.291 | 87.383 | 9.626 | 1.00 | 49.35 | O |
| ATOM | 890 | N | ASP | A | 58 | −29.343 | 85.927 | 8.205 | 1.00 | 49.47 | N |
| ATOM | 892 | CA | ASP | A | 58 | −28.983 | 86.947 | 7.249 | 1.00 | 45.77 | C |
| ATOM | 894 | CB | ASP | A | 58 | −27.915 | 86.413 | 6.299 | 1.00 | 47.00 | C |
| ATOM | 897 | CG | ASP | A | 58 | −26.630 | 86.018 | 7.009 | 1.00 | 38.80 | C |
| ATOM | 898 | OD1 | ASP | A | 58 | −26.288 | 84.805 | 7.091 | 1.00 | 53.03 | O |
| ATOM | 899 | OD2 | ASP | A | 58 | −25.875 | 86.875 | 7.492 | 1.00 | 59.02 | O |
| ATOM | 900 | C | ASP | A | 58 | −30.187 | 87.535 | 6.494 | 1.00 | 44.83 | C |
| ATOM | 901 | O | ASP | A | 58 | −30.119 | 88.646 | 5.980 | 1.00 | 45.72 | O |
| ATOM | 902 | N | THR | A | 59 | −31.280 | 86.792 | 6.396 | 1.00 | 44.32 | N |
| ATOM | 904 | CA | THR | A | 59 | −32.448 | 87.255 | 5.648 | 1.00 | 44.03 | C |
| ATOM | 906 | CB | THR | A | 59 | −32.354 | 86.841 | 4.179 | 1.00 | 43.48 | C |
| ATOM | 908 | OG1 | THR | A | 59 | −31.420 | 87.674 | 3.483 | 1.00 | 55.41 | O |
| ATOM | 910 | CG2 | THR | A | 59 | −33.663 | 87.041 | 3.485 | 1.00 | 48.14 | C |
| ATOM | 914 | C | THR | A | 59 | −33.624 | 86.532 | 6.240 | 1.00 | 41.84 | C |
| ATOM | 915 | O | THR | A | 59 | −33.580 | 85.328 | 6.353 | 1.00 | 51.92 | O |
| ATOM | 916 | N | TYR | A | 60 | −34.637 | 87.255 | 6.677 | 1.00 | 41.34 | N |
| ATOM | 918 | CA | TYR | A | 60 | −35.733 | 86.666 | 7.419 | 1.00 | 41.20 | C |
| ATOM | 920 | CB | TYR | A | 60 | −35.539 | 86.813 | 8.948 | 1.00 | 43.52 | C |
| ATOM | 923 | CG | TYR | A | 60 | −36.469 | 85.889 | 9.722 | 1.00 | 48.22 | C |
| ATOM | 924 | CD1 | TYR | A | 60 | −36.123 | 84.555 | 9.991 | 1.00 | 50.68 | C |
| ATOM | 926 | CE1 | TYR | A | 60 | −37.037 | 83.673 | 10.595 | 1.00 | 43.46 | C |
| ATOM | 928 | CZ | TYR | A | 60 | −38.309 | 84.122 | 10.903 | 1.00 | 57.61 | C |
| ATOM | 929 | OH | TYR | A | 60 | −39.245 | 83.328 | 11.525 | 1.00 | 53.12 | O |
| ATOM | 931 | CE2 | TYR | A | 60 | −38.681 | 85.412 | 10.593 | 1.00 | 55.39 | C |
| ATOM | 933 | CD2 | TYR | A | 60 | −37.769 | 86.280 | 10.002 | 1.00 | 43.82 | C |
| ATOM | 935 | C | TYR | A | 60 | −36.985 | 87.379 | 6.915 | 1.00 | 45.68 | C |
| ATOM | 936 | O | TYR | A | 60 | −36.905 | 88.507 | 6.446 | 1.00 | 61.33 | O |
| ATOM | 937 | N | ARG | A | 61 | −38.116 | 86.692 | 6.934 | 1.00 | 43.07 | N |
| ATOM | 939 | CA | ARG | A | 61 | −39.334 | 87.128 | 6.280 | 1.00 | 37.91 | C |
| ATOM | 941 | CB | ARG | A | 61 | −39.205 | 87.174 | 4.766 | 1.00 | 35.58 | C |
| ATOM | 944 | CG | ARG | A | 61 | −40.565 | 86.933 | 4.051 | 1.00 | 52.12 | C |
| ATOM | 947 | CD | ARG | A | 61 | −40.616 | 87.449 | 2.626 | 1.00 | 53.19 | C |
| ATOM | 950 | NE | ARG | A | 61 | −41.959 | 87.449 | 2.067 | 1.00 | 52.47 | N |
| ATOM | 952 | CZ | ARG | A | 61 | −42.204 | 87.219 | 0.783 | 1.00 | 63.95 | C |
| ATOM | 953 | NH1 | ARG | A | 61 | −41.195 | 86.973 | −0.051 | 1.00 | 60.72 | N |
| ATOM | 956 | NH2 | ARG | A | 61 | −43.453 | 87.262 | 0.320 | 1.00 | 44.73 | N |
| ATOM | 959 | C | ARG | A | 61 | −40.428 | 86.117 | 6.590 | 1.00 | 45.77 | C |
| ATOM | 960 | O | ARG | A | 61 | −40.392 | 84.948 | 6.156 | 1.00 | 48.56 | O |
| ATOM | 961 | N | CYS | A | 62 | −41.422 | 86.599 | 7.317 | 1.00 | 45.29 | N |
| ATOM | 963 | CA | CYS | A | 62 | −42.519 | 85.761 | 7.700 | 1.00 | 49.99 | C |
| ATOM | 965 | CB | CYS | A | 62 | −42.285 | 85.319 | 9.140 | 1.00 | 46.38 | C |
| ATOM | 968 | SG | CYS | A | 62 | −43.571 | 84.149 | 9.614 | 1.00 | 52.07 | S |
| ATOM | 969 | C | CYS | A | 62 | −43.868 | 86.468 | 7.573 | 1.00 | 50.35 | C |
| ATOM | 970 | O | CYS | A | 62 | −44.367 | 87.058 | 8.530 | 1.00 | 61.52 | O |
| ATOM | 971 | N | ASP | A | 63 | −44.498 | 86.389 | 6.415 | 1.00 | 48.72 | N |
| ATOM | 973 | CA | ASP | A | 63 | −45.744 | 87.130 | 6.248 | 1.00 | 49.68 | C |
| ATOM | 975 | CB | ASP | A | 63 | −46.347 | 86.980 | 4.847 | 1.00 | 43.35 | C |
| ATOM | 978 | CG | ASP | A | 63 | −45.378 | 87.358 | 3.736 | 1.00 | 45.49 | C |
| ATOM | 979 | OD1 | ASP | A | 63 | −44.544 | 88.292 | 3.865 | 1.00 | 56.65 | O |
| ATOM | 980 | OD2 | ASP | A | 63 | −45.403 | 86.745 | 2.647 | 1.00 | 61.50 | O |
| ATOM | 981 | C | ASP | A | 63 | −46.749 | 86.661 | 7.285 | 1.00 | 54.07 | C |
| ATOM | 982 | O | ASP | A | 63 | −47.264 | 87.478 | 8.025 | 1.00 | 68.79 | O |
| ATOM | 983 | N | ARG | A | 64 | −47.051 | 85.367 | 7.315 | 1.00 | 56.70 | N |
| ATOM | 985 | CA | ARG | A | 64 | −48.061 | 84.815 | 8.205 | 1.00 | 56.46 | C |
| ATOM | 987 | CB | ARG | A | 64 | −49.260 | 84.325 | 7.378 | 1.00 | 57.67 | C |
| ATOM | 990 | CG | ARG | A | 64 | −50.258 | 83.424 | 8.135 | 1.00 | 87.57 | C |
| ATOM | 993 | CD | ARG | A | 64 | −51.725 | 83.938 | 8.205 | 1.00 | 105.79 | C |
| ATOM | 996 | NE | ARG | A | 64 | −52.158 | 84.729 | 7.046 | 1.00 | 106.80 | N |
| ATOM | 998 | CZ | ARG | A | 64 | −53.135 | 85.637 | 7.074 | 1.00 | 104.94 | C |
| ATOM | 999 | NH1 | ARG | A | 64 | −53.804 | 85.876 | 8.196 | 1.00 | 109.12 | N |
| ATOM | 1002 | NH2 | ARG | A | 64 | −53.448 | 86.321 | 5.981 | 1.00 | 93.49 | N |
| ATOM | 1005 | C | ARG | A | 64 | −47.429 | 83.686 | 9.038 | 1.00 | 56.33 | C |
| ATOM | 1006 | O | ARG | A | 64 | −46.726 | 82.810 | 8.503 | 1.00 | 59.75 | O |
| ATOM | 1007 | N | ASN | A | 65 | −47.648 | 83.738 | 10.351 | 1.00 | 47.16 | N |
| ATOM | 1009 | CA | ASN | A | 65 | −47.150 | 82.722 | 11.275 | 1.00 | 43.82 | C |
| ATOM | 1011 | CB | ASN | A | 65 | −47.711 | 82.960 | 12.673 | 1.00 | 41.38 | C |
| ATOM | 1014 | CG | ASN | A | 65 | −47.136 | 84.187 | 13.362 | 1.00 | 49.59 | C |
| ATOM | 1015 | OD1 | ASN | A | 65 | −47.466 | 84.439 | 14.512 | 1.00 | 63.06 | O |
| ATOM | 1016 | ND2 | ASN | A | 65 | −46.267 | 84.937 | 12.691 | 1.00 | 66.39 | N |
| ATOM | 1019 | C | ASN | A | 65 | −47.503 | 81.290 | 10.859 | 1.00 | 45.94 | C |
| ATOM | 1020 | O | ASN | A | 65 | −48.590 | 81.021 | 10.373 | 1.00 | 51.30 | O |
| ATOM | 1021 | N | LEU | A | 66 | −46.580 | 80.368 | 11.095 | 1.00 | 51.79 | N |
| ATOM | 1023 | CA | LEU | A | 66 | −46.684 | 78.995 | 10.614 | 1.00 | 57.13 | C |
| ATOM | 1025 | CB | LEU | A | 66 | −45.612 | 78.672 | 9.581 | 1.00 | 57.68 | C |
| ATOM | 1028 | CG | LEU | A | 66 | −45.725 | 79.079 | 8.119 | 1.00 | 71.18 | C |
| ATOM | 1030 | CD1 | LEU | A | 66 | −44.374 | 79.654 | 7.727 | 1.00 | 72.51 | C |
| ATOM | 1034 | CD2 | LEU | A | 66 | −46.074 | 77.887 | 7.225 | 1.00 | 71.50 | C |
| ATOM | 1038 | C | LEU | A | 66 | −46.438 | 78.036 | 11.765 | 1.00 | 49.92 | C |
| ATOM | 1039 | O | LEU | A | 66 | −45.586 | 78.246 | 12.602 | 1.00 | 49.45 | O |
| ATOM | 1040 | N | ALA | A | 67 | −47.133 | 76.918 | 11.750 | 1.00 | 47.85 | N |
| ATOM | 1042 | CA | ALA | A | 67 | −46.666 | 75.814 | 12.533 | 1.00 | 48.60 | C |
| ATOM | 1044 | CB | ALA | A | 67 | −47.665 | 75.523 | 13.619 | 1.00 | 39.49 | C |
| ATOM | 1048 | C | ALA | A | 67 | −46.469 | 74.622 | 11.593 | 1.00 | 49.29 | C |
| ATOM | 1049 | O | ALA | A | 67 | −47.443 | 73.951 | 11.270 | 1.00 | 54.30 | O |
| ATOM | 1050 | N | MET | A | 68 | −45.228 | 74.398 | 11.146 | 1.00 | 45.99 | N |
| ATOM | 1052 | CA | MET | A | 68 | −44.914 | 73.280 | 10.256 | 1.00 | 53.21 | C |
| ATOM | 1054 | CB | MET | A | 68 | −44.006 | 73.630 | 9.056 | 1.00 | 49.26 | C |
| ATOM | 1057 | CG | MET | A | 68 | −43.050 | 74.752 | 9.294 | 1.00 | 51.45 | C |
| ATOM | 1060 | SD | MET | A | 68 | −42.135 | 75.135 | 7.791 | 1.00 | 58.25 | S |
| ATOM | 1061 | CE | MET | A | 68 | −40.476 | 74.852 | 8.436 | 1.00 | 63.26 | C |
| ATOM | 1065 | C | MET | A | 68 | −44.319 | 72.092 | 10.990 | 1.00 | 48.01 | C |
| ATOM | 1066 | O | MET | A | 68 | −43.471 | 72.229 | 11.869 | 1.00 | 44.98 | O |
| ATOM | 1067 | N | GLY | A | 69 | −44.784 | 70.922 | 10.581 | 1.00 | 40.55 | N |
| ATOM | 1069 | CA | GLY | A | 69 | −44.614 | 69.757 | 11.387 | 1.00 | 38.21 | C |
| ATOM | 1072 | C | GLY | A | 69 | −43.688 | 69.006 | 10.479 | 1.00 | 45.83 | C |
| ATOM | 1073 | O | GLY | A | 69 | −43.893 | 68.921 | 9.259 | 1.00 | 42.26 | O |
| ATOM | 1074 | N | VAL | A | 70 | −42.624 | 68.521 | 11.099 | 1.00 | 41.99 | N |
| ATOM | 1076 | CA | VAL | A | 70 | −41.437 | 68.214 | 10.365 | 1.00 | 40.73 | C |
| ATOM | 1078 | CB | VAL | A | 70 | −40.327 | 69.177 | 10.678 | 1.00 | 41.15 | C |
| ATOM | 1080 | CG1 | VAL | A | 70 | −39.080 | 60.650 | 10.005 | 1.00 | 43.34 | C |
| ATOM | 1084 | CG2 | VAL | A | 70 | −40.678 | 70.593 | 10.245 | 1.00 | 47.04 | C |
| ATOM | 1088 | C | VAL | A | 70 | −40.965 | 66.892 | 10.906 | 1.00 | 40.84 | C |
| ATOM | 1089 | O | VAL | A | 70 | −40.798 | 66.769 | 12.097 | 1.00 | 38.48 | O |
| ATOM | 1090 | N | ASN | A | 71 | −40.741 | 65.920 | 10.033 | 1.00 | 47.30 | N |
| ATOM | 1092 | CA | ASN | A | 71 | −39.938 | 64.772 | 10.380 | 1.00 | 50.58 | C |
| ATOM | 1094 | CB | ASN | A | 71 | −40.103 | 63.676 | 9.338 | 1.00 | 53.54 | C |
| ATOM | 1097 | CG | ASN | A | 71 | −39.422 | 62.402 | 9.766 | 1.00 | 55.12 | C |
| ATOM | 1098 | OD1 | ASN | A | 71 | −38.249 | 62.402 | 10.131 | 1.00 | 60.08 | O |
| ATOM | 1099 | ND2 | ASN | A | 71 | −40.172 | 61.328 | 9.815 | 1.00 | 62.57 | N |
| ATOM | 1102 | C | ASN | A | 71 | −38.453 | 65.108 | 10.490 | 1.00 | 53.92 | C |
| ATOM | 1103 | O | ASN | A | 71 | −37.803 | 65.429 | 9.486 | 1.00 | 58.66 | O |
| ATOM | 1104 | N | LEU | A | 72 | −37.894 | 64.981 | 11.690 | 1.00 | 47.91 | N |
| ATOM | 1106 | CA | LEU | A | 72 | −36.581 | 65.566 | 11.941 | 1.00 | 49.55 | C |
| ATOM | 1108 | CB | LEU | A | 72 | −36.284 | 65.740 | 13.430 | 1.00 | 51.20 | C |
| ATOM | 1111 | CG | LEU | A | 72 | −37.220 | 66.660 | 14.217 | 1.00 | 38.32 | C |
| ATOM | 1113 | CD1 | LEU | A | 72 | −36.409 | 67.256 | 15.334 | 1.00 | 55.83 | C |
| ATOM | 1117 | CD2 | LEU | A | 72 | −37.783 | 67.730 | 13.336 | 1.00 | 57.23 | C |
| ATOM | 1121 | C | LEU | A | 72 | −35.532 | 64.672 | 11.320 | 1.00 | 48.72 | C |
| ATOM | 1122 | O | LEU | A | 72 | −34.474 | 65.124 | 10.895 | 1.00 | 58.64 | O |
| ATOM | 1123 | N | THR | A | 73 | −35.857 | 63.398 | 11.225 | 1.00 | 41.96 | N |
| ATOM | 1125 | CA | THR | A | 73 | −34.978 | 62.484 | 10.558 | 1.00 | 42.22 | C |
| ATOM | 1127 | CB | THR | A | 73 | −35.436 | 61.097 | 10.796 | 1.00 | 42.18 | C |
| ATOM | 1129 | OG1 | THR | A | 73 | −35.389 | 60.841 | 12.202 | 1.00 | 53.97 | O |
| ATOM | 1131 | CG2 | THR | A | 73 | −34.467 | 60.176 | 10.180 | 1.00 | 46.49 | C |
| ATOM | 1135 | C | THR | A | 73 | −34.930 | 62.779 | 9.083 | 1.00 | 42.35 | C |
| ATOM | 1136 | O | THR | A | 73 | −33.853 | 62.860 | 8.502 | 1.00 | 47.32 | O |
| ATOM | 1137 | N | SER | A | 74 | −36.083 | 63.028 | 8.488 | 1.00 | 42.43 | N |
| ATOM | 1139 | CA | SER | A | 74 | −36.094 | 63.495 | 7.104 | 1.00 | 44.17 | C |
| ATOM | 1141 | CB | SER | A | 74 | −37.514 | 63.795 | 6.652 | 1.00 | 37.38 | C |
| ATOM | 1144 | OG | SER | A | 74 | −30.332 | 62.654 | 6.720 | 1.00 | 52.47 | O |
| ATOM | 1146 | C | SER | A | 74 | −35.253 | 64.763 | 6.944 | 1.00 | 45.87 | C |
| ATOM | 1147 | O | SER | A | 74 | −34.500 | 64.934 | 5.962 | 1.00 | 44.84 | O |
| ATOM | 1148 | N | MET | A | 75 | −35.430 | 65.683 | 7.888 | 1.00 | 38.23 | N |
| ATOM | 1150 | CA | MET | A | 75 | −34.764 | 66.958 | 7.732 | 1.00 | 39.62 | C |
| ATOM | 1152 | CB | MET | A | 75 | −35.255 | 67.972 | 8.766 | 1.00 | 45.53 | C |
| ATOM | 1155 | CG | MET | A | 75 | −35.168 | 69.410 | 8.307 | 1.00 | 24.89 | C |
| ATOM | 1158 | SD | MET | A | 75 | −35.795 | 70.493 | 9.596 | 1.00 | 39.70 | S |
| ATOM | 1159 | CE | MET | A | 75 | −34.921 | 71.913 | 8.912 | 1.00 | 25.42 | C |
| ATOM | 1163 | C | MET | A | 75 | −33.258 | 66.753 | 7.880 | 1.00 | 40.73 | C |
| ATOM | 1164 | O | MET | A | 75 | −32.522 | 67.355 | 7.129 | 1.00 | 39.58 | O |
| ATOM | 1165 | N | SER | A | 76 | −32.794 | 65.944 | 8.836 | 1.00 | 39.20 | N |
| ATOM | 1167 | CA | SER | A | 76 | −31.359 | 65.753 | 8.976 | 1.00 | 47.55 | C |
| ATOM | 1169 | CB | SER | A | 76 | −30.909 | 65.034 | 10.258 | 1.00 | 54.41 | C |
| ATOM | 1172 | OG | SER | A | 76 | −31.522 | 63.775 | 10.445 | 1.00 | 73.82 | O |
| ATOM | 1174 | C | SER | A | 76 | −30.792 | 65.078 | 7.744 | 1.00 | 40.88 | C |
| ATOM | 1175 | O | SER | A | 76 | −29.690 | 65.400 | 7.312 | 1.00 | 35.64 | O |
| ATOM | 1176 | N | LYS | A | 77 | −31.580 | 64.220 | 7.119 | 1.00 | 37.45 | N |
| ATOM | 1178 | CA | LYS | A | 77 | −31.041 | 63.467 | 6.004 | 1.00 | 38.37 | C |
| ATOM | 1180 | CB | LYS | A | 77 | −32.042 | 62.431 | 5.495 | 1.00 | 40.54 | C |
| ATOM | 1183 | CG | LYS | A | 77 | −31.835 | 60.997 | 5.951 | 1.00 | 43.97 | C |
| ATOM | 1186 | CD | LYS | A | 77 | −32.876 | 60.047 | 5.336 | 1.00 | 66.06 | C |
| ATOM | 1189 | CE | LYS | A | 77 | −33.482 | 59.100 | 6.380 | 1.00 | 75.07 | C |
| ATOM | 1192 | NZ | LYS | A | 77 | −34.521 | 58.232 | 5.774 | 1.00 | 66.50 | N |
| ATOM | 1196 | C | LYS | A | 77 | −30.770 | 64.466 | 4.904 | 1.00 | 37.45 | C |
| ATOM | 1197 | O | LYS | A | 77 | −29.804 | 64.338 | 4.169 | 1.00 | 49.46 | O |
| ATOM | 1198 | N | ILE | A | 78 | −31.673 | 65.415 | 4.742 | 1.00 | 33.50 | N |
| ATOM | 1200 | CA | ILE | A | 78 | −31.482 | 66.462 | 3.755 | 1.00 | 35.83 | C |
| ATOM | 1202 | CB | ILE | A | 78 | −32.826 | 67.234 | 3.583 | 1.00 | 32.82 | C |
| ATOM | 1204 | CG1 | ILE | A | 78 | −33.865 | 66.278 | 2.989 | 1.00 | 40.18 | C |
| ATOM | 1207 | CD1 | ILE | A | 78 | −35.226 | 66.853 | 2.849 | 1.00 | 24.88 | C |
| ATOM | 1211 | CG2 | ILE | A | 78 | −32.650 | 68.491 | 2.710 | 1.00 | 42.94 | C |
| ATOM | 1215 | C | ILE | A | 78 | −30.300 | 67.378 | 4.134 | 1.00 | 36.01 | C |
| ATOM | 1216 | O | ILE | A | 78 | −29.561 | 67.885 | 3.275 | 1.00 | 32.19 | O |
| ATOM | 1217 | N | LEU | A | 79 | −30.148 | 67.652 | 5.423 | 1.00 | 38.48 | N |
| ATOM | 1219 | CA | LEU | A | 79 | −29.236 | 68.720 | 5.798 | 1.00 | 41.70 | C |
| ATOM | 1221 | CB | LEU | A | 79 | −29.534 | 69.292 | 7.165 | 1.00 | 34.89 | C |
| ATOM | 1224 | CG | LEU | A | 79 | −30.496 | 70.486 | 7.137 | 1.00 | 39.85 | C |
| ATOM | 1226 | CD1 | LEU | A | 79 | −30.679 | 71.034 | 8.534 | 1.00 | 53.93 | C |
| ATOM | 1230 | CD2 | LEU | A | 79 | −29.989 | 71.616 | 6.312 | 1.00 | 48.63 | C |
| ATOM | 1234 | C | LEU | A | 79 | −27.812 | 68.201 | 5.719 | 1.00 | 45.47 | C |
| ATOM | 1235 | O | LEU | A | 79 | −26.881 | 68.993 | 5.555 | 1.00 | 32.50 | O |
| ATOM | 1236 | N | LYS | A | 80 | −27.680 | 66.871 | 5.715 | 1.00 | 43.97 | N |
| ATOM | 1238 | CA | LYS | A | 80 | −26.393 | 66.206 | 5.525 | 1.00 | 31.93 | C |
| ATOM | 1240 | CB | LYS | A | 80 | −26.393 | 64.737 | 5.976 | 1.00 | 32.33 | C |
| ATOM | 1243 | CG | LYS | A | 80 | −26.413 | 64.564 | 7.498 | 1.00 | 46.41 | C |
| ATOM | 1246 | CD | LYS | A | 80 | −26.505 | 63.107 | 7.977 | 1.00 | 80.96 | C |
| ATOM | 1249 | CE | LYS | A | 80 | −25.832 | 62.909 | 9.340 | 1.00 | 87.69 | C |
| ATOM | 1252 | NZ | LYS | A | 80 | −24.352 | 63.162 | 9.237 | 1.00 | 91.71 | N |
| ATOM | 1256 | C | LYS | A | 80 | −26.009 | 66.259 | 4.081 | 1.00 | 33.31 | C |
| ATOM | 1257 | O | LYS | A | 80 | −24.866 | 65.963 | 3.769 | 1.00 | 37.14 | O |
| ATOM | 1258 | N | CYS | A | 81 | −26.894 | 66.699 | 3.187 | 1.00 | 35.64 | N |
| ATOM | 1260 | CA | CYS | A | 81 | −26.410 | 67.033 | 1.845 | 1.00 | 36.01 | C |
| ATOM | 1262 | CB | CYS | A | 81 | −27.467 | 66.787 | 0.782 | 1.00 | 37.20 | C |
| ATOM | 1265 | SG | CYS | A | 81 | −28.383 | 65.263 | 0.926 | 1.00 | 49.35 | S |
| ATOM | 1266 | C | CYS | A | 81 | −25.959 | 68.485 | 1.700 | 1.00 | 34.37 | C |
| ATOM | 1267 | O | CYS | A | 81 | −25.828 | 69.006 | 0.597 | 1.00 | 44.01 | O |
| ATOM | 1268 | N | ALA | A | 82 | −25.784 | 69.199 | 2.793 | 1.00 | 37.43 | N |
| ATOM | 1270 | CA | ALA | A | 82 | −25.264 | 70.554 | 2.659 | 1.00 | 41.97 | C |
| ATOM | 1272 | CB | ALA | A | 82 | −26.030 | 71.550 | 3.509 | 1.00 | 39.15 | C |
| ATOM | 1276 | C | ALA | A | 82 | −23.846 | 70.438 | 3.147 | 1.00 | 41.03 | C |
| ATOM | 1277 | O | ALA | A | 82 | −23.583 | 69.661 | 4.059 | 1.00 | 44.95 | O |
| ATOM | 1278 | N | GLY | A | 83 | −22.950 | 71.165 | 2.490 | 1.00 | 37.61 | N |
| ATOM | 1280 | CA | GLY | A | 83 | −21.686 | 71.561 | 3.067 | 1.00 | 35.39 | C |
| ATOM | 1283 | C | GLY | A | 83 | −21.685 | 72.494 | 4.256 | 1.00 | 38.63 | C |
| ATOM | 1284 | O | GLY | A | 83 | −22.648 | 73.218 | 4.481 | 1.00 | 45.69 | O |
| ATOM | 1285 | N | ASN | A | 84 | −20.588 | 72.476 | 5.010 | 1.00 | 43.95 | N |
| ATOM | 1287 | CA | ASN | A | 84 | −20.464 | 73.315 | 6.192 | 1.00 | 49.73 | C |
| ATOM | 1289 | CB | ASN | A | 84 | −19.343 | 72.817 | 7.104 | 1.00 | 55.15 | C |
| ATOM | 1292 | CG | ASN | A | 84 | −19.590 | 71.418 | 7.618 | 1.00 | 53.93 | C |
| ATOM | 1293 | OD1 | ASN | A | 84 | −20.341 | 71.226 | 8.575 | 1.00 | 59.28 | O |
| ATOM | 1294 | ND2 | ASN | A | 84 | −18.984 | 70.431 | 6.965 | 1.00 | 69.76 | N |
| ATOM | 1297 | C | ASN | A | 84 | −20.226 | 74.761 | 5.783 | 1.00 | 47.58 | C |
| ATOM | 1298 | O | ASN | A | 84 | −20.629 | 75.698 | 6.463 | 1.00 | 46.22 | O |
| ATOM | 1299 | N | GLU | A | 85 | −19.694 | 74.941 | 4.590 | 1.00 | 46.90 | N |
| ATOM | 1301 | CA | GLU | A | 85 | −19.585 | 76.287 | 4.098 | 1.00 | 53.59 | C |
| ATOM | 1303 | CB | GLU | A | 85 | −18.291 | 76.387 | 3.271 | 1.00 | 63.16 | C |
| ATOM | 1306 | CG | GLU | A | 85 | −17.018 | 76.437 | 4.120 | 1.00 | 71.99 | C |
| ATOM | 1309 | CD | GLU | A | 85 | −17.298 | 76.799 | 5.578 | 1.00 | 89.14 | C |
| ATOM | 1310 | OE1 | GLU | A | 85 | −17.525 | 78.000 | 5.883 | 1.00 | 78.41 | O |
| ATOM | 1311 | OE2 | GLU | A | 85 | −17.312 | 75.876 | 6.425 | 1.00 | 97.32 | O |
| ATOM | 1312 | C | GLU | A | 85 | −20.870 | 76.833 | 3.417 | 1.00 | 44.19 | C |
| ATOM | 1313 | O | GLU | A | 85 | −21.013 | 78.027 | 3.140 | 1.00 | 41.11 | O |
| ATOM | 1314 | N | ASP | A | 86 | −21.844 | 75.968 | 3.204 | 1.00 | 44.05 | N |
| ATOM | 1316 | CA | ASP | A | 86 | −22.978 | 76.293 | 2.343 | 1.00 | 39.86 | C |
| ATOM | 1318 | CB | ASP | A | 86 | −23.831 | 75.042 | 2.085 | 1.00 | 35.44 | C |
| ATOM | 1321 | CG | ASP | A | 86 | −23.204 | 74.143 | 1.068 | 1.00 | 39.66 | C |
| ATOM | 1322 | OD1 | ASP | A | 86 | −22.102 | 74.516 | 0.582 | 1.00 | 44.47 | O |
| ATOM | 1323 | OD2 | ASP | A | 86 | −23.731 | 73.060 | 0.719 | 1.00 | 45.10 | O |
| ATOM | 1324 | C | ASP | A | 86 | −23.831 | 77.376 | 2.984 | 1.00 | 40.74 | C |
| ATOM | 1325 | O | ASP | A | 86 | −23.896 | 77.522 | 4.202 | 1.00 | 36.77 | O |
| ATOM | 1326 | N | ILE | A | 87 | −24.489 | 78.126 | 2.116 | 1.00 | 46.48 | N |
| ATOM | 1328 | CA | ILE | A | 87 | −25.478 | 79.126 | 2.482 | 1.00 | 47.17 | C |
| ATOM | 1330 | CB | ILE | A | 87 | −25.499 | 80.238 | 1.424 | 1.00 | 47.21 | C |
| ATOM | 1332 | CG1 | ILE | A | 87 | −24.100 | 80.618 | 0.894 | 1.00 | 48.58 | C |
| ATOM | 1335 | CD1 | ILE | A | 87 | −23.481 | 81.787 | 1.566 | 1.00 | 62.44 | C |
| ATOM | 1339 | CG2 | ILE | A | 87 | −26.431 | 81.333 | 1.853 | 1.00 | 41.97 | C |
| ATOM | 1343 | C | ILE | A | 87 | −26.813 | 78.414 | 2.336 | 1.00 | 49.11 | C |
| ATOM | 1344 | O | ILE | A | 87 | −27.141 | 77.977 | 1.223 | 1.00 | 38.70 | O |
| ATOM | 1345 | N | ILE | A | 88 | −27.591 | 78.377 | 3.419 | 1.00 | 46.32 | N |
| ATOM | 1347 | CA | ILE | A | 88 | −28.816 | 77.596 | 3.451 | 1.00 | 45.19 | C |
| ATOM | 1349 | CB | ILE | A | 88 | −28.868 | 76.695 | 4.682 | 1.00 | 47.75 | C |
| ATOM | 1351 | CG1 | ILE | A | 88 | −27.763 | 75.644 | 4.595 | 1.00 | 52.94 | C |
| ATOM | 1354 | CD1 | ILE | A | 88 | −27.542 | 74.908 | 5.886 | 1.00 | 67.63 | C |
| ATOM | 1358 | CG2 | ILE | A | 88 | −30.217 | 76.028 | 4.727 | 1.00 | 41.84 | C |
| ATOM | 1362 | C | ILE | A | 88 | −30.010 | 78.486 | 3.520 | 1.00 | 40.63 | C |
| ATOM | 1363 | O | ILE | A | 88 | −30.161 | 79.203 | 4.491 | 1.00 | 46.13 | O |
| ATOM | 1364 | N | THR | A | 89 | −30.917 | 78.319 | 2.571 | 1.00 | 44.06 | N |
| ATOM | 1366 | CA | THR | A | 89 | −32.218 | 78.968 | 2.626 | 1.00 | 45.86 | C |
| ATOM | 1368 | CB | THR | A | 89 | −32.414 | 79.745 | 1.350 | 1.00 | 44.18 | C |
| ATOM | 1370 | OG1 | THR | A | 89 | −31.199 | 80.445 | 1.076 | 1.00 | 47.35 | O |
| ATOM | 1372 | CG2 | THR | A | 89 | −33.434 | 80.874 | 1.555 | 1.00 | 53.20 | C |
| ATOM | 1376 | C | THR | A | 89 | −33.408 | 78.038 | 2.846 | 1.00 | 47.80 | C |
| ATOM | 1377 | O | THR | A | 89 | −33.543 | 77.024 | 2.162 | 1.00 | 55.10 | O |
| ATOM | 1378 | N | LEU | A | 90 | −34.224 | 78.388 | 3.844 | 1.00 | 46.35 | N |
| ATOM | 1380 | CA | LEU | A | 90 | −35.513 | 77.771 | 4.135 | 1.00 | 43.14 | C |
| ATOM | 1382 | CB | LEU | A | 90 | −35.691 | 77.729 | 5.646 | 1.00 | 32.58 | C |
| ATOM | 1385 | CG | LEU | A | 90 | −34.626 | 76.904 | 6.364 | 1.00 | 36.37 | C |
| ATOM | 1387 | CD1 | LEU | A | 90 | −34.864 | 76.788 | 7.871 | 1.00 | 41.84 | C |
| ATOM | 1391 | CD2 | LEU | A | 90 | −34.417 | 75.526 | 5.766 | 1.00 | 41.63 | C |
| ATOM | 1395 | C | LEU | A | 90 | −36.638 | 78.610 | 3.526 | 1.00 | 47.97 | C |
| ATOM | 1396 | O | LEU | A | 90 | −36.721 | 79.812 | 3.798 | 1.00 | 52.39 | O |
| ATOM | 1397 | N | ARG | A | 91 | −37.493 | 77.994 | 2.707 | 1.00 | 48.21 | N |
| ATOM | 1399 | CA | ARG | A | 91 | −38.693 | 78.657 | 2.190 | 1.00 | 47.37 | C |
| ATOM | 1401 | CB | ARG | A | 91 | −30.460 | 79.001 | 0.723 | 1.00 | 53.64 | C |
| ATOM | 1404 | CG | ARG | A | 91 | −39.437 | 80.002 | 0.107 | 1.00 | 63.21 | C |
| ATOM | 1407 | CD | ARG | A | 91 | −39.906 | 79.672 | −1.328 | 1.00 | 84.97 | C |
| ATOM | 1410 | NE | ARG | A | 91 | −39.017 | 78.774 | −2.075 | 1.00 | 98.93 | N |
| ATOM | 1412 | CZ | ARG | A | 91 | −37.738 | 79.021 | −2.357 | 1.00 | 101.01 | C |
| ATOM | 1413 | NH1 | ARG | A | 91 | −37.044 | 78.123 | −3.041 | 1.00 | 88.07 | N |
| ATOM | 1416 | NH2 | ARG | A | 91 | −37.146 | 80.143 | −1.950 | 1.00 | 106.38 | N |
| ATOM | 1419 | C | ARG | A | 91 | −39.960 | 77.809 | 2.347 | 1.00 | 46.90 | C |
| ATOM | 1420 | O | ARG | A | 91 | −39.975 | 76.625 | 2.043 | 1.00 | 44.59 | O |
| ATOM | 1421 | N | ALA | A | 92 | −41.042 | 78.379 | 2.862 | 1.00 | 53.34 | N |
| ATOM | 1423 | CA | ALA | A | 92 | −42.347 | 77.703 | 2.757 | 1.00 | 52.03 | C |
| ATOM | 1425 | CB | ALA | A | 92 | −42.760 | 77.121 | 4.079 | 1.00 | 46.35 | C |
| ATOM | 1429 | C | ALA | A | 92 | −43.411 | 78.677 | 2.308 | 1.00 | 53.19 | C |
| ATOM | 1430 | O | ALA | A | 92 | −43.430 | 79.845 | 2.724 | 1.00 | 55.12 | O |
| ATOM | 1431 | N | GLU | A | 93 | −44.327 | 78.170 | 1.496 | 1.00 | 55.86 | N |
| ATOM | 1433 | CA | GLU | A | 93 | −45.583 | 78.869 | 1.246 | 1.00 | 56.96 | C |
| ATOM | 1435 | CB | GLU | A | 93 | −46.274 | 78.262 | 0.025 | 1.00 | 56.69 | C |
| ATOM | 1438 | CG | GLU | A | 93 | −45.640 | 78.644 | −1.309 | 1.00 | 61.63 | C |
| ATOM | 1441 | CD | GLU | A | 93 | −45.529 | 80.149 | −1.474 | 1.00 | 95.67 | C |
| ATOM | 1442 | OE1 | GLU | A | 93 | −46.582 | 80.830 | −1.395 | 1.00 | 102.05 | O |
| ATOM | 1443 | OE2 | GLU | A | 93 | −44.389 | 80.646 | −1.639 | 1.00 | 110.05 | O |
| ATOM | 1444 | C | GLU | A | 93 | −46.486 | 78.870 | 2.482 | 1.00 | 54.84 | C |
| ATOM | 1445 | O | GLU | A | 93 | −48.097 | 78.452 | 3.563 | 1.00 | 59.94 | O |
| ATOM | 1446 | N | ASP | A | 94 | −47.656 | 79.472 | 2.390 | 1.00 | 71.55 | N |
| ATOM | 1448 | CA | ASP | A | 94 | −48.325 | 79.872 | 3.625 | 1.00 | 79.77 | C |
| ATOM | 1450 | CB | ASP | A | 94 | −49.196 | 81.113 | 3.430 | 1.00 | 76.79 | C |
| ATOM | 1453 | CG | ASP | A | 94 | −48.452 | 82.379 | 3.776 | 1.00 | 84.19 | C |
| ATOM | 1454 | OD1 | ASP | A | 94 | −47.823 | 82.388 | 4.853 | 1.00 | 105.41 | O |
| ATOM | 1455 | OD2 | ASP | A | 94 | −48.383 | 83.381 | 3.030 | 1.00 | 100.72 | O |
| ATOM | 1456 | C | ASP | A | 94 | −49.097 | 78.707 | 4.228 | 1.00 | 82.92 | C |
| ATOM | 1457 | O | ASP | A | 94 | −48.973 | 78.435 | 5.425 | 1.00 | 74.35 | O |
| ATOM | 1458 | N | ASN | A | 95 | −49.795 | 77.963 | 3.374 | 1.00 | 86.38 | N |
| ATOM | 1460 | CA | ASN | A | 95 | −50.211 | 76.612 | 3.732 | 1.00 | 93.81 | C |
| ATOM | 1462 | CB | ASN | A | 95 | −51.524 | 76.270 | 3.030 | 1.00 | 98.04 | C |
| ATOM | 1465 | CG | ASN | A | 95 | −52.682 | 77.130 | 3.518 | 1.00 | 102.30 | C |
| ATOM | 1466 | OD1 | ASN | A | 95 | −53.030 | 77.108 | 4.699 | 1.00 | 94.34 | O |
| ATOM | 1467 | ND2 | ASN | A | 95 | −53.263 | 77.913 | 2.614 | 1.00 | 116.89 | N |
| ATOM | 1470 | C | ASN | A | 95 | −49.169 | 75.523 | 3.488 | 1.00 | 91.68 | C |
| ATOM | 1471 | O | ASN | A | 95 | −49.050 | 74.584 | 4.275 | 1.00 | 96.84 | O |
| ATOM | 1472 | N | ALA | A | 96 | −48.410 | 75.646 | 2.405 | 1.00 | 91.04 | N |
| ATOM | 1474 | CA | ALA | A | 96 | −48.062 | 74.473 | 1.609 | 1.00 | 89.51 | C |
| ATOM | 1476 | CB | ALA | A | 96 | −47.041 | 74.781 | 0.474 | 1.00 | 88.82 | C |
| ATOM | 1480 | C | ALA | A | 96 | −47.543 | 73.367 | 2.519 | 1.00 | 82.41 | C |
| ATOM | 1481 | O | ALA | A | 96 | −47.269 | 73.571 | 3.716 | 1.00 | 65.70 | O |
| ATOM | 1482 | N | ASP | A | 97 | −47.492 | 72.180 | 1.923 | 1.00 | 75.06 | N |
| ATOM | 1484 | CA | ASP | A | 97 | −47.323 | 70.982 | 2.694 | 1.00 | 76.55 | C |
| ATOM | 1486 | CB | ASP | A | 97 | −48.434 | 69.980 | 2.388 | 1.00 | 79.10 | C |
| ATOM | 1489 | CG | ASP | A | 97 | −49.546 | 70.032 | 3.453 | 1.00 | 103.31 | C |
| ATOM | 1490 | OD1 | ASP | A | 97 | −50.457 | 70.891 | 3.289 | 1.00 | 107.61 | O |
| ATOM | 1491 | OD2 | ASP | A | 97 | −49.535 | 69.335 | 4.515 | 1.00 | 101.16 | O |
| ATOM | 1492 | C | ASP | A | 97 | −45.898 | 70.535 | 2.442 | 1.00 | 67.88 | C |
| ATOM | 1493 | O | ASP | A | 97 | −45.540 | 69.366 | 2.495 | 1.00 | 70.80 | O |
| ATOM | 1494 | N | THR | A | 98 | −45.059 | 71.545 | 2.273 | 1.00 | 60.04 | N |
| ATOM | 1496 | CA | THR | A | 98 | −43.709 | 71.348 | 1.795 | 1.00 | 52.32 | C |
| ATOM | 1498 | CB | THR | A | 98 | −43.745 | 71.326 | 0.279 | 1.00 | 52.73 | C |
| ATOM | 1500 | OG1 | THR | A | 98 | −43.432 | 69.995 | −0.147 | 1.00 | 40.67 | O |
| ATOM | 1502 | CG2 | THR | A | 98 | −42.696 | 72.260 | −0.378 | 1.00 | 62.63 | C |
| ATOM | 1506 | C | THR | A | 98 | −42.775 | 72.421 | 2.334 | 1.00 | 49.23 | C |
| ATOM | 1507 | O | THR | A | 98 | −43.201 | 73.564 | 2.529 | 1.00 | 50.70 | O |
| ATOM | 1508 | N | LEU | A | 99 | −41.544 | 72.022 | 2.664 | 1.00 | 39.28 | N |
| ATOM | 1510 | CA | LEU | A | 99 | −40.476 | 72.976 | 2.933 | 1.00 | 35.80 | C |
| ATOM | 1512 | CB | LEU | A | 99 | −39.852 | 72.716 | 4.294 | 1.00 | 36.56 | C |
| ATOM | 1515 | CG | LEU | A | 99 | −38.819 | 73.758 | 4.748 | 1.00 | 37.23 | C |
| ATOM | 1517 | CD1 | LEU | A | 99 | −39.496 | 75.068 | 4.985 | 1.00 | 46.31 | C |
| ATOM | 1521 | CD2 | LEU | A | 99 | −38.107 | 73.373 | 6.050 | 1.00 | 41.08 | C |
| ATOM | 1525 | C | LEU | A | 99 | −39.393 | 72.792 | 1.925 | 1.00 | 36.96 | C |
| ATOM | 1526 | O | LEU | A | 99 | −38.896 | 71.681 | 1.747 | 1.00 | 47.62 | O |
| ATOM | 1527 | N | ALA | A | 100 | −38.982 | 73.894 | 1.330 | 1.00 | 38.65 | N |
| ATOM | 1529 | CA | ALA | A | 100 | −37.811 | 73.914 | 0.479 | 1.00 | 45.04 | C |
| ATOM | 1531 | CB | ALA | A | 100 | −38.014 | 74.908 | −0.628 | 1.00 | 46.86 | C |
| ATOM | 1535 | C | ALA | A | 100 | −36.571 | 74.281 | 1.271 | 1.00 | 45.93 | C |
| ATOM | 1536 | O | ALA | A | 100 | −36.611 | 75.253 | 2.026 | 1.00 | 41.27 | O |
| ATOM | 1537 | N | LEU | A | 101 | −35.500 | 73.490 | 1.109 | 1.00 | 48.44 | N |
| ATOM | 1539 | CA | LEU | A | 101 | −34.167 | 73.876 | 1.577 | 1.00 | 43.72 | C |
| ATOM | 1541 | CB | LEU | A | 101 | −33.578 | 72.792 | 2.472 | 1.00 | 43.22 | C |
| ATOM | 1544 | CG | LEU | A | 101 | −34.579 | 72.506 | 3.593 | 1.00 | 38.29 | C |
| ATOM | 1546 | CD1 | LEU | A | 101 | −35.516 | 71.423 | 3.162 | 1.00 | 31.87 | C |
| ATOM | 1550 | CD2 | LEU | A | 101 | −33.904 | 72.161 | 4.878 | 1.00 | 32.56 | C |
| ATOM | 1554 | C | LEU | A | 101 | −33.290 | 74.055 | 0.367 | 1.00 | 46.08 | C |
| ATOM | 1555 | O | LEU | A | 101 | −33.153 | 73.122 | −0.438 | 1.00 | 49.79 | O |
| ATOM | 1556 | N | VAL | A | 102 | −32.730 | 75.255 | 0.233 | 1.00 | 40.57 | N |
| ATOM | 1558 | CA | VAL | A | 102 | −31.776 | 75.534 | −0.828 | 1.00 | 44.54 | C |
| ATOM | 1560 | CB | VAL | A | 102 | −32.165 | 76.848 | −1.501 | 1.00 | 43.88 | C |
| ATOM | 1562 | CG1 | VAL | A | 102 | −31.172 | 77.260 | −2.559 | 1.00 | 43.56 | C |
| ATOM | 1566 | CG2 | VAL | A | 102 | −33.497 | 76.689 | −2.151 | 1.00 | 50.27 | C |
| ATOM | 1570 | C | VAL | A | 102 | −30.378 | 75.669 | −0.237 | 1.00 | 43.88 | C |
| ATOM | 1571 | O | VAL | A | 102 | −30.214 | 76.353 | 0.759 | 1.00 | 44.64 | O |
| ATOM | 1572 | N | PHE | A | 103 | −29.396 | 74.983 | −0.804 | 1.00 | 41.19 | N |
| ATOM | 1574 | CA | PHE | A | 103 | −28.009 | 75.122 | −0.358 | 1.00 | 43.62 | C |
| ATOM | 1576 | CB | PHE | A | 103 | −27.430 | 73.780 | 0.075 | 1.00 | 41.69 | C |
| ATOM | 1579 | CG | PHE | A | 103 | −28.333 | 72.933 | 0.925 | 1.00 | 50.83 | C |
| ATOM | 1580 | CD1 | PHE | A | 103 | −29.014 | 73.480 | 1.993 | 1.00 | 44.50 | C |
| ATOM | 1582 | CE1 | PHE | A | 103 | −29.738 | 72.652 | 2.862 | 1.00 | 46.73 | C |
| ATOM | 1584 | CZ | PHE | A | 103 | −29.786 | 71.277 | 2.647 | 1.00 | 30.52 | C |
| ATOM | 1586 | CE2 | PHE | A | 103 | −29.121 | 70.733 | 1.572 | 1.00 | 41.47 | C |
| ATOM | 1588 | CD2 | PHE | A | 103 | −28.387 | 71.549 | 0.732 | 1.00 | 50.27 | C |
| ATOM | 1590 | C | PHE | A | 103 | −27.112 | 75.608 | −1.506 | 1.00 | 45.03 | C |
| ATOM | 1591 | O | PHE | A | 103 | −27.041 | 74.948 | −2.551 | 1.00 | 45.05 | O |
| ATOM | 1592 | N | GLU | A | 104 | −26.369 | 76.694 | −1.288 | 1.00 | 41.09 | N |
| ATOM | 1594 | CA | GLU | A | 104 | −25.427 | 77.175 | −2.288 | 1.00 | 44.94 | C |
| ATOM | 1596 | CB | GLU | A | 104 | −25.706 | 78.605 | −2.735 | 1.00 | 47.82 | C |
| ATOM | 1599 | CG | GLU | A | 104 | −27.116 | 79.026 | −2.419 | 1.00 | 58.95 | C |
| ATOM | 1602 | CD | GLU | A | 104 | −27.705 | 79.949 | −3.446 | 1.00 | 67.56 | C |
| ATOM | 1603 | OE1 | GLU | A | 104 | −27.037 | 80.183 | −4.501 | 1.00 | 64.25 | O |
| ATOM | 1604 | OE2 | GLU | A | 104 | −28.830 | 80.411 | −3.111 | 1.00 | 61.00 | O |
| ATOM | 1605 | C | GLU | A | 104 | −24.063 | 77.193 | −1.695 | 1.00 | 40.24 | C |
| ATOM | 1606 | O | GLU | A | 104 | −23.860 | 77.713 | −0.630 | 1.00 | 50.49 | O |
| ATOM | 1607 | N | ALA | A | 105 | −23.112 | 76.680 | −2.442 | 1.00 | 47.92 | N |
| ATOM | 1609 | CA | ALA | A | 105 | −21.706 | 76.939 | −2.229 | 1.00 | 44.83 | C |
| ATOM | 1611 | CB | ALA | A | 105 | −20.944 | 76.195 | −3.277 | 1.00 | 48.93 | C |
| ATOM | 1615 | C | ALA | A | 105 | −21.407 | 78.415 | −2.358 | 1.00 | 52.50 | C |
| ATOM | 1616 | O | ALA | A | 105 | −21.723 | 79.030 | −3.397 | 1.00 | 48.88 | O |
| ATOM | 1617 | N | PRO | A | 106 | −20.711 | 78.943 | −1.351 | 1.00 | 58.11 | N |
| ATOM | 1618 | CA | PRO | A | 106 | −20.149 | 80.299 | −1.418 | 1.00 | 69.07 | C |
| ATOM | 1620 | CB | PRO | A | 106 | −19.303 | 80.401 | −0.140 | 1.00 | 70.28 | C |
| ATOM | 1623 | CG | PRO | A | 106 | −19.045 | 78.968 | 0.288 | 1.00 | 65.48 | C |
| ATOM | 1626 | CD | PRO | A | 106 | −20.282 | 78.234 | −0.137 | 1.00 | 59.06 | C |
| ATOM | 1629 | C | PRO | A | 106 | −19.271 | 80.426 | −2.664 | 1.00 | 72.17 | C |
| ATOM | 1630 | O | PRO | A | 106 | −19.275 | 81.432 | −3.401 | 1.00 | 80.04 | O |
| ATOM | 1631 | N | ASN | A | 107 | −18.513 | 79.362 | −2.889 | 1.00 | 70.75 | N |
| ATOM | 1633 | CA | ASN | A | 107 | −17.291 | 79.490 | −3.641 | 1.00 | 72.37 | C |
| ATOM | 1635 | CB | ASN | A | 107 | −16.104 | 79.718 | −2.692 | 1.00 | 77.34 | C |
| ATOM | 1638 | CG | ASN | A | 107 | −16.145 | 81.114 | −2.018 | 1.00 | 91.81 | C |
| ATOM | 1639 | OD1 | ASN | A | 107 | −16.073 | 82.152 | −2.690 | 1.00 | 99.24 | O |
| ATOM | 1640 | ND2 | ASN | A | 107 | −16.283 | 81.134 | −0.690 | 1.00 | 97.95 | N |
| ATOM | 1643 | C | ASN | A | 107 | −17.137 | 78.340 | −4.633 | 1.00 | 66.13 | C |
| ATOM | 1644 | O | ASN | A | 107 | −16.037 | 77.869 | −4.900 | 1.00 | 69.04 | O |
| ATOM | 1645 | N | GLN | A | 108 | −18.251 | 78.013 | −5.279 | 1.00 | 57.45 | N |
| ATOM | 1647 | CA | GLN | A | 108 | −18.288 | 77.064 | −6.379 | 1.00 | 60.85 | C |
| ATOM | 1649 | CB | GLN | A | 108 | −18.092 | 75.662 | −5.799 | 1.00 | 69.10 | C |
| ATOM | 1652 | CG | GLN | A | 108 | −17.474 | 74.604 | −6.723 | 1.00 | 84.05 | C |
| ATOM | 1655 | CD | GLN | A | 108 | −17.466 | 73.251 | −6.035 | 1.00 | 94.19 | C |
| ATOM | 1656 | OE1 | GLN | A | 108 | −17.753 | 73.184 | −4.835 | 1.00 | 84.96 | O |
| ATOM | 1657 | NE2 | GLN | A | 108 | −17.141 | 72.186 | −6.773 | 1.00 | 95.30 | N |
| ATOM | 1660 | C | GLN | A | 108 | −19.665 | 77.140 | −7.059 | 1.00 | 62.09 | C |
| ATOM | 1661 | O | GLN | A | 108 | −20.679 | 77.359 | −6.397 | 1.00 | 59.79 | O |
| ATOM | 1662 | N | GLU | A | 109 | −19.747 | 76.902 | −8.362 | 1.00 | 58.28 | N |
| ATOM | 1664 | CA | GLU | A | 109 | −21.056 | 76.922 | −8.997 | 1.00 | 57.51 | C |
| ATOM | 1666 | CB | GLU | A | 109 | −20.902 | 77.281 | −10.471 | 1.00 | 69.86 | C |
| ATOM | 1669 | CG | GLU | A | 109 | −19.723 | 78.213 | −10.762 | 1.00 | 80.51 | C |
| ATOM | 1672 | CD | GLU | A | 109 | −20.091 | 79.403 | −11.637 | 1.00 | 89.49 | C |
| ATOM | 1673 | OE1 | GLU | A | 109 | −20.908 | 79.258 | −12.576 | 1.00 | 100.19 | O |
| ATOM | 1674 | OE2 | GLU | A | 109 | −19.540 | 80.496 | −11.396 | 1.00 | 85.36 | O |
| ATOM | 1675 | C | GLU | A | 109 | −21.835 | 75.619 | −8.781 | 1.00 | 52.42 | C |
| ATOM | 1676 | O | GLU | A | 109 | −22.006 | 74.761 | −9.674 | 1.00 | 50.60 | O |
| ATOM | 1677 | N | LYS | A | 110 | −22.314 | 75.490 | −7.550 | 1.00 | 44.61 | N |
| ATOM | 1679 | CA | LYS | A | 110 | −23.095 | 74.328 | −7.137 | 1.00 | 42.92 | C |
| ATOM | 1681 | CB | LYS | A | 110 | −22.256 | 73.339 | −6.305 | 1.00 | 46.49 | C |
| ATOM | 1684 | CG | LYS | A | 110 | −23.039 | 72.177 | −5.623 | 1.00 | 41.57 | C |
| ATOM | 1687 | CD | LYS | A | 110 | −22.083 | 71.072 | −5.118 | 1.00 | 45.69 | C |
| ATOM | 1690 | CE | LYS | A | 110 | −22.374 | 70.476 | −3.737 | 1.00 | 36.77 | C |
| ATOM | 1693 | NZ | LYS | A | 110 | −22.920 | 69.105 | −3.871 | 1.00 | 46.26 | N |
| ATOM | 1697 | C | LYS | A | 110 | −24.270 | 74.828 | −6.317 | 1.00 | 37.58 | C |
| ATOM | 1698 | O | LYS | A | 110 | −24.082 | 75.605 | −5.401 | 1.00 | 38.73 | O |
| ATOM | 1699 | N | VAL | A | 111 | −25.474 | 74.394 | −6.664 | 1.00 | 37.05 | N |
| ATOM | 1701 | CA | VAL | A | 111 | −26.688 | 74.848 | −6.006 | 1.00 | 44.74 | C |
| ATOM | 1703 | CB | VAL | A | 111 | −27.443 | 76.003 | −6.757 | 1.00 | 41.14 | C |
| ATOM | 1705 | CG1 | VAL | A | 111 | −28.567 | 76.541 | −5.872 | 1.00 | 39.88 | C |
| ATOM | 1709 | CG2 | VAL | A | 111 | −26.530 | 77.159 | −7.061 | 1.00 | 53.95 | C |
| ATOM | 1713 | C | VAL | A | 111 | −27.622 | 73.653 | −5.831 | 1.00 | 42.94 | C |
| ATOM | 1714 | O | VAL | A | 111 | −27.914 | 72.914 | −6.763 | 1.00 | 46.90 | O |
| ATOM | 1715 | N | SER | A | 112 | −28.093 | 73.465 | −4.612 | 1.00 | 42.97 | N |
| ATOM | 1717 | CA | SER | A | 112 | −28.865 | 72.281 | −4.315 | 1.00 | 44.85 | C |
| ATOM | 1719 | CB | SER | A | 112 | −28.142 | 71.414 | −3.262 | 1.00 | 44.11 | C |
| ATOM | 1722 | OG | SER | A | 112 | −26.991 | 70.759 | −3.801 | 1.00 | 50.85 | O |
| ATOM | 1724 | C | SER | A | 112 | −30.259 | 72.738 | −3.874 | 1.00 | 44.76 | C |
| ATOM | 1725 | O | SER | A | 112 | −30.444 | 73.725 | −3.130 | 1.00 | 40.21 | O |
| ATOM | 1726 | N | ASP | A | 113 | −31.261 | 71.995 | −4.320 | 1.00 | 38.87 | N |
| ATOM | 1728 | CA | ASP | A | 113 | −32.628 | 72.331 | −3.957 | 1.00 | 43.73 | C |
| ATOM | 1730 | CB | ASP | A | 113 | −33.396 | 72.910 | −5.151 | 1.00 | 49.06 | C |
| ATOM | 1733 | CG | ASP | A | 113 | −32.770 | 74.161 | −5.690 | 1.00 | 61.65 | C |
| ATOM | 1734 | OD1 | ASP | A | 113 | −32.927 | 75.205 | −5.017 | 1.00 | 87.75 | O |
| ATOM | 1735 | OD2 | ASP | A | 113 | −32.125 | 74.183 | −6.767 | 1.00 | 87.35 | O |
| ATOM | 1736 | C | ASP | A | 113 | −33.310 | 71.056 | −3.519 | 1.00 | 39.88 | C |
| ATOM | 1737 | O | ASP | A | 113 | −33.409 | 70.058 | −4.267 | 1.00 | 40.34 | O |
| ATOM | 1738 | N | TYR | A | 114 | −33.851 | 71.115 | −2.319 | 1.00 | 35.13 | N |
| ATOM | 1740 | CA | TYR | A | 114 | −34.526 | 69.939 | −1.831 | 1.00 | 38.73 | C |
| ATOM | 1742 | CB | TYR | A | 114 | −33.755 | 69.367 | −0.661 | 1.00 | 31.17 | C |
| ATOM | 1745 | CG | TYR | A | 114 | −32.479 | 68.708 | −1.088 | 1.00 | 31.02 | C |
| ATOM | 1746 | CD1 | TYR | A | 114 | −32.445 | 67.345 | −1.346 | 1.00 | 52.38 | C |
| ATOM | 1748 | CE1 | TYR | A | 114 | −31.270 | 66.714 | −1.698 | 1.00 | 41.87 | C |
| ATOM | 1750 | CZ | TYR | A | 114 | −30.113 | 67.439 | −1.747 | 1.00 | 36.57 | C |
| ATOM | 1751 | OH | TYR | A | 114 | −28.947 | 66.805 | −2.080 | 1.00 | 61.65 | O |
| ATOM | 1753 | CE2 | TYR | A | 114 | −30.107 | 68.788 | −1.481 | 1.00 | 38.58 | C |
| ATOM | 1755 | CD2 | TYR | A | 114 | −31.293 | 69.422 | −1.183 | 1.00 | 23.82 | C |
| ATOM | 1757 | C | TYR | A | 114 | −35.898 | 70.352 | −1.408 | 1.00 | 37.20 | C |
| ATOM | 1758 | O | TYR | A | 114 | −36.074 | 71.442 | −0.874 | 1.00 | 37.14 | O |
| ATOM | 1759 | N | GLU | A | 115 | −36.841 | 69.438 | −1.549 | 1.00 | 37.23 | N |
| ATOM | 1761 | CA | GLU | A | 115 | −38.207 | 69.724 | −1.120 | 1.00 | 43.23 | C |
| ATOM | 1763 | CB | GLU | A | 115 | −39.095 | 69.678 | −2.347 | 1.00 | 43.86 | C |
| ATOM | 1766 | CG | GLU | A | 115 | −40.089 | 70.811 | −2.430 | 1.00 | 67.78 | C |
| ATOM | 1769 | CD | GLU | A | 115 | −41.130 | 70.492 | −3.462 | 1.00 | 61.02 | C |
| ATOM | 1770 | OE1 | GLU | A | 115 | −42.056 | 69.727 | −3.152 | 1.00 | 54.85 | O |
| ATOM | 1771 | OE2 | GLU | A | 115 | −40.931 | 70.908 | −4.607 | 1.00 | 76.84 | O |
| ATOM | 1772 | C | GLU | A | 115 | −38.704 | 68.690 | −0.099 | 1.00 | 43.84 | C |
| ATOM | 1773 | O | GLU | A | 115 | −38.816 | 67.484 | −0.383 | 1.00 | 40.31 | O |
| ATOM | 1774 | N | MET | A | 116 | −38.971 | 69.142 | 1.118 | 1.00 | 42.26 | N |
| ATOM | 1776 | CA | MET | A | 116 | −39.235 | 68.198 | 2.199 | 1.00 | 40.55 | C |
| ATOM | 1778 | CB | MET | A | 116 | −38.604 | 68.689 | 3.508 | 1.00 | 40.38 | C |
| ATOM | 1781 | CG | MET | A | 116 | −38.811 | 67.678 | 4.629 | 1.00 | 35.04 | C |
| ATOM | 1784 | SD | MET | A | 116 | −37.971 | 68.050 | 6.167 | 1.00 | 51.53 | S |
| ATOM | 1785 | CE | MET | A | 116 | −38.095 | 69.901 | 6.479 | 1.00 | 41.00 | C |
| ATOM | 1789 | C | MET | A | 116 | −40.728 | 68.126 | 2.402 | 1.00 | 37.42 | C |
| ATOM | 1790 | O | MET | A | 116 | −41.347 | 69.123 | 2.752 | 1.00 | 45.68 | O |
| ATOM | 1791 | N | LYS | A | 117 | −41.323 | 66.967 | 2.183 | 1.00 | 36.89 | N |
| ATOM | 1793 | CA | LYS | A | 117 | −42.709 | 66.769 | 2.587 | 1.00 | 40.77 | C |
| ATOM | 1795 | CB | LYS | A | 117 | −43.143 | 65.347 | 2.247 | 1.00 | 47.65 | C |
| ATOM | 1798 | CG | LYS | A | 117 | −42.989 | 64.940 | 0.743 | 1.00 | 66.90 | C |
| ATOM | 1801 | CD | LYS | A | 117 | −43.913 | 63.732 | 0.391 | 1.00 | 75.56 | C |
| ATOM | 1804 | CE | LYS | A | 117 | −43.344 | 62.715 | −0.605 | 1.00 | 62.45 | C |
| ATOM | 1807 | NZ | LYS | A | 117 | −44.061 | 62.824 | −1.899 | 1.00 | 80.59 | N |
| ATOM | 1811 | C | LYS | A | 117 | −42.847 | 67.020 | 4.080 | 1.00 | 36.28 | C |
| ATOM | 1812 | O | LYS | A | 117 | −42.024 | 66.550 | 4.838 | 1.00 | 47.78 | O |
| ATOM | 1813 | N | LEU | A | 118 | −43.829 | 67.809 | 4.503 | 1.00 | 42.00 | N |
| ATOM | 1815 | CA | LEU | A | 118 | −44.064 | 68.061 | 5.923 | 1.00 | 45.52 | C |
| ATOM | 1817 | CB | LEU | A | 118 | −44.539 | 69.487 | 6.186 | 1.00 | 44.25 | C |
| ATOM | 1820 | CG | LEU | A | 118 | −43.685 | 70.657 | 5.737 | 1.00 | 41.45 | C |
| ATOM | 1822 | CD1 | LEU | A | 118 | −44.600 | 71.850 | 5.801 | 1.00 | 55.10 | C |
| ATOM | 1826 | CD2 | LEU | A | 118 | −42.462 | 70.872 | 6.601 | 1.00 | 47.31 | C |
| ATOM | 1830 | C | LEU | A | 118 | −45.152 | 67.118 | 6.405 | 1.00 | 48.29 | C |
| ATOM | 1831 | O | LEU | A | 118 | −45.656 | 66.330 | 5.608 | 1.00 | 43.19 | O |
| ATOM | 1832 | N | MET | A | 119 | −45.465 | 67.167 | 7.704 | 1.00 | 49.32 | N |
| ATOM | 1834 | CA | MET | A | 119 | −46.485 | 66.295 | 8.249 | 1.00 | 48.21 | C |
| ATOM | 1836 | CB | MET | A | 119 | −45.877 | 65.077 | 8.931 | 1.00 | 58.98 | C |
| ATOM | 1839 | CG | MET | A | 119 | −45.194 | 65.326 | 10.272 | 1.00 | 63.14 | C |
| ATOM | 1842 | SD | MET | A | 119 | −44.073 | 63.957 | 10.696 | 1.00 | 75.91 | S |
| ATOM | 1843 | CE | MET | A | 119 | −45.207 | 62.769 | 11.298 | 1.00 | 75.17 | C |
| ATOM | 1847 | C | MET | A | 119 | −47.455 | 66.996 | 9.171 | 1.00 | 52.56 | C |
| ATOM | 1848 | O | MET | A | 119 | −47.158 | 68.023 | 9.779 | 1.00 | 58.04 | O |
| ATOM | 1849 | N | ASP | A | 120 | −48.648 | 66.424 | 9.242 | 1.00 | 54.26 | N |
| ATOM | 1851 | CA | ASP | A | 120 | −49.724 | 66.988 | 10.022 | 1.00 | 54.80 | C |
| ATOM | 1853 | CB | ASP | A | 120 | −51.000 | 66.214 | 9.695 | 1.00 | 59.87 | C |
| ATOM | 1856 | CG | ASP | A | 120 | −52.213 | 67.071 | 9.830 | 1.00 | 59.58 | C |
| ATOM | 1857 | OD1 | ASP | A | 120 | −52.120 | 68.102 | 10.552 | 1.00 | 43.50 | O |
| ATOM | 1858 | OD2 | ASP | A | 120 | −53.245 | 66.824 | 9.181 | 1.00 | 69.77 | O |
| ATOM | 1859 | C | ASP | A | 120 | −49.446 | 66.862 | 11.512 | 1.00 | 54.33 | C |
| ATOM | 1860 | O | ASP | A | 120 | −49.534 | 65.762 | 12.048 | 1.00 | 40.59 | O |
| ATOM | 1861 | N | LEU | A | 121 | −49.131 | 67.955 | 12.202 | 1.00 | 60.58 | N |
| ATOM | 1863 | CA | LEU | A | 121 | −48.770 | 67.763 | 13.596 | 1.00 | 65.88 | C |
| ATOM | 1865 | CB | LEU | A | 121 | −47.322 | 68.134 | 13.930 | 1.00 | 63.79 | C |
| ATOM | 1868 | CG | LEU | A | 121 | −46.354 | 66.996 | 13.598 | 1.00 | 49.52 | C |
| ATOM | 1870 | CD1 | LEU | A | 121 | −44.891 | 67.202 | 14.041 | 1.00 | 64.73 | C |
| ATOM | 1874 | CD2 | LEU | A | 121 | −46.849 | 65.669 | 14.108 | 1.00 | 48.09 | C |
| ATOM | 1878 | C | LEU | A | 121 | −49.767 | 68.199 | 14.647 | 1.00 | 70.28 | C |
| ATOM | 1879 | O | LEU | A | 121 | −50.433 | 67.350 | 15.221 | 1.00 | 88.49 | O |
| ATOM | 1880 | N | ASP | A | 122 | −49.798 | 69.451 | 15.049 | 1.00 | 72.20 | N |
| ATOM | 1882 | CA | ASP | A | 122 | −50.666 | 69.757 | 16.181 | 1.00 | 76.43 | C |
| ATOM | 1884 | CB | ASP | A | 122 | −52.040 | 69.107 | 15.903 | 1.00 | 77.98 | C |
| ATOM | 1887 | CG | ASP | A | 122 | −53.156 | 69.687 | 16.752 | 1.00 | 77.15 | C |
| ATOM | 1888 | OD1 | ASP | A | 122 | −54.145 | 68.983 | 17.085 | 1.00 | 80.35 | O |
| ATOM | 1889 | OD2 | ASP | A | 122 | −53.095 | 70.870 | 17.129 | 1.00 | 69.23 | O |
| ATOM | 1890 | C | ASP | A | 122 | −50.166 | 69.227 | 17.529 | 1.00 | 69.42 | C |
| ATOM | 1891 | O | ASP | A | 122 | −50.826 | 68.361 | 18.083 | 1.00 | 68.77 | O |
| ATOM | 1892 | N | VAL | A | 123 | −49.100 | 69.772 | 18.115 | 1.00 | 65.82 | N |
| ATOM | 1894 | CA | VAL | A | 123 | −48.619 | 69.216 | 19.384 | 1.00 | 66.68 | C |
| ATOM | 1896 | CB | VAL | A | 123 | −47.118 | 69.392 | 19.605 | 1.00 | 64.43 | C |
| ATOM | 1898 | CG1 | VAL | A | 123 | −46.324 | 68.347 | 18.875 | 1.00 | 62.58 | C |
| ATOM | 1902 | CG2 | VAL | A | 123 | −46.694 | 70.784 | 19.167 | 1.00 | 87.18 | C |
| ATOM | 1906 | C | VAL | A | 123 | −49.303 | 69.719 | 20.655 | 1.00 | 71.14 | C |
| ATOM | 1907 | O | VAL | A | 123 | −49.737 | 68.912 | 21.493 | 1.00 | 78.46 | O |
| ATOM | 1908 | N | GLU | A | 124 | −49.325 | 71.025 | 20.886 | 1.00 | 66.22 | N |
| ATOM | 1910 | CA | GLU | A | 124 | −49.757 | 71.524 | 22.207 | 1.00 | 72.78 | C |
| ATOM | 1912 | CB | GLU | A | 124 | −50.655 | 70.575 | 23.031 | 1.00 | 73.63 | C |
| ATOM | 1915 | CG | GLU | A | 124 | −51.117 | 71.263 | 24.327 | 1.00 | 87.86 | C |
| ATOM | 1918 | CD | GLU | A | 124 | −52.004 | 70.437 | 25.261 | 1.00 | 94.32 | C |
| ATOM | 1919 | OE1 | GLU | A | 124 | −53.172 | 70.848 | 25.509 | 1.00 | 76.39 | O |
| ATOM | 1920 | OE2 | GLU | A | 124 | −51.497 | 69.435 | 25.822 | 1.00 | 78.38 | O |
| ATOM | 1921 | C | GLU | A | 124 | −48.619 | 71.912 | 23.129 | 1.00 | 65.17 | C |
| ATOM | 1922 | O | GLU | A | 124 | −48.091 | 71.087 | 23.865 | 1.00 | 61.50 | O |
| ATOM | 1923 | N | GLN | A | 125 | −48.349 | 73.207 | 23.156 | 1.00 | 61.41 | N |
| ATOM | 1925 | CA | GLN | A | 125 | −47.271 | 73.744 | 23.941 | 1.00 | 66.61 | C |
| ATOM | 1927 | CB | GLN | A | 125 | −47.122 | 75.222 | 23.625 | 1.00 | 70.42 | C |
| ATOM | 1930 | CG | GLN | A | 125 | −46.941 | 75.465 | 22.133 | 1.00 | 86.17 | C |
| ATOM | 1933 | CD | GLN | A | 125 | −45.546 | 75.940 | 21.818 | 1.00 | 88.45 | C |
| ATOM | 1934 | OE1 | GLN | A | 125 | −45.192 | 76.093 | 20.647 | 1.00 | 102.53 | O |
| ATOM | 1935 | NE2 | GLN | A | 125 | −44.749 | 76.188 | 22.864 | 1.00 | 75.83 | N |
| ATOM | 1938 | C | GLN | A | 125 | −47.549 | 73.522 | 25.416 | 1.00 | 66.84 | C |
| ATOM | 1939 | O | GLN | A | 125 | −48.619 | 73.835 | 25.926 | 1.00 | 73.77 | O |
| ATOM | 1940 | N | LEU | A | 126 | −46.582 | 72.904 | 26.071 | 1.00 | 64.91 | N |
| ATOM | 1942 | CA | LEU | A | 126 | −46.433 | 72.979 | 27.504 | 1.00 | 63.08 | C |
| ATOM | 1944 | CB | LEU | A | 126 | −45.427 | 71.918 | 27.941 | 1.00 | 68.81 | C |
| ATOM | 1947 | CG | LEU | A | 126 | −45.957 | 70.495 | 27.991 | 1.00 | 69.60 | C |
| ATOM | 1949 | CD1 | LEU | A | 126 | −45.997 | 69.832 | 26.612 | 1.00 | 81.31 | C |
| ATOM | 1953 | CD2 | LEU | A | 126 | −45.016 | 69.788 | 28.939 | 1.00 | 80.78 | C |
| ATOM | 1957 | C | LEU | A | 126 | −45.859 | 74.331 | 27.873 | 1.00 | 58.80 | C |
| ATOM | 1958 | O | LEU | A | 126 | −45.269 | 75.003 | 27.053 | 1.00 | 47.52 | O |
| ATOM | 1959 | N | GLY | A | 127 | −45.950 | 74.697 | 29.140 | 1.00 | 61.05 | N |
| ATOM | 1961 | CA | GLY | A | 127 | −45.530 | 76.023 | 29.524 | 1.00 | 61.08 | C |
| ATOM | 1964 | C | GLY | A | 127 | −44.487 | 75.852 | 30.592 | 1.00 | 63.35 | C |
| ATOM | 1965 | O | GLY | A | 127 | −44.626 | 74.978 | 31.437 | 1.00 | 65.15 | O |
| ATOM | 1966 | N | ILE | A | 128 | −43.461 | 76.694 | 30.536 | 1.00 | 66.30 | N |
| ATOM | 1968 | CA | ILE | A | 128 | −42.227 | 76.476 | 31.269 | 1.00 | 66.41 | C |
| ATOM | 1970 | CB | ILE | A | 128 | −40.989 | 76.460 | 30.314 | 1.00 | 61.35 | C |
| ATOM | 1972 | CG1 | ILE | A | 128 | −40.809 | 75.117 | 29.604 | 1.00 | 49.73 | C |
| ATOM | 1975 | CD1 | ILE | A | 128 | −40.557 | 75.238 | 28.100 | 1.00 | 58.65 | C |
| ATOM | 1979 | CG2 | ILE | A | 128 | −39.731 | 76.678 | 31.082 | 1.00 | 54.60 | C |
| ATOM | 1983 | C | ILE | A | 128 | −42.170 | 77.666 | 32.213 | 1.00 | 76.79 | C |
| ATOM | 1984 | O | ILE | A | 128 | −41.691 | 78.750 | 31.851 | 1.00 | 74.11 | O |
| ATOM | 1985 | N | PRO | A | 129 | −42.700 | 77.476 | 33.415 | 1.00 | 84.50 | N |
| ATOM | 1986 | CA | PRO | A | 129 | −42.600 | 78.508 | 34.445 | 1.00 | 85.00 | C |
| ATOM | 1988 | CB | PRO | A | 129 | −42.883 | 77.759 | 35.756 | 1.00 | 91.17 | C |
| ATOM | 1991 | CG | PRO | A | 129 | −43.165 | 76.294 | 35.395 | 1.00 | 92.42 | C |
| ATOM | 1994 | CD | PRO | A | 129 | −43.376 | 76.262 | 33.904 | 1.00 | 89.15 | C |
| ATOM | 1997 | C | PRO | A | 129 | −41.165 | 78.943 | 34.388 | 1.00 | 83.68 | C |
| ATOM | 1998 | O | PRO | A | 129 | −40.318 | 78.088 | 34.168 | 1.00 | 77.34 | O |
| ATOM | 1999 | N | GLU | A | 130 | −40.921 | 80.241 | 34.461 | 1.00 | 90.37 | N |
| ATOM | 2001 | CA | GLU | A | 130 | −39.564 | 80.757 | 34.389 | 1.00 | 95.80 | C |
| ATOM | 2003 | CB | GLU | A | 130 | −39.484 | 81.935 | 33.409 | 1.00 | 98.30 | C |
| ATOM | 2006 | CG | GLU | A | 130 | −38.077 | 82.452 | 33.149 | 1.00 | 111.97 | C |
| ATOM | 2009 | CD | GLU | A | 130 | −37.925 | 83.897 | 33.591 | 1.00 | 129.56 | C |
| ATOM | 2010 | OE1 | GLU | A | 130 | −38.954 | 84.507 | 33.951 | 1.00 | 137.95 | O |
| ATOM | 2011 | OE2 | GLU | A | 130 | −36.792 | 84.428 | 33.582 | 1.00 | 131.42 | O |
| ATOM | 2012 | C | GLU | A | 130 | −39.206 | 81.180 | 35.806 | 1.00 | 89.07 | C |
| ATOM | 2013 | O | GLU | A | 130 | −40.017 | 81.789 | 36.484 | 1.00 | 82.03 | O |
| ATOM | 2014 | N | GLN | A | 131 | −38.019 | 80.804 | 36.266 | 1.00 | 86.50 | N |
| ATOM | 2016 | CA | GLN | A | 131 | −37.764 | 80.711 | 37.693 | 1.00 | 82.67 | C |
| ATOM | 2018 | CB | GLN | A | 131 | −38.362 | 79.411 | 38.230 | 1.00 | 84.21 | C |
| ATOM | 2021 | CG | GLN | A | 131 | −37.911 | 78.172 | 37.461 | 1.00 | 83.70 | C |
| ATOM | 2024 | CD | GLN | A | 131 | −37.758 | 76.942 | 38.358 | 1.00 | 93.30 | C |
| ATOM | 2025 | OE1 | GLN | A | 131 | −37.198 | 77.020 | 39.458 | 1.00 | 96.06 | O |
| ATOM | 2026 | NE2 | GLN | A | 131 | −38.242 | 75.800 | 37.881 | 1.00 | 77.13 | N |
| ATOM | 2029 | C | GLN | A | 131 | −36.276 | 80.787 | 38.028 | 1.00 | 75.94 | C |
| ATOM | 2030 | O | GLN | A | 131 | −35.427 | 81.054 | 37.181 | 1.00 | 66.12 | O |
| ATOM | 2031 | N | GLU | A | 132 | −35.973 | 80.579 | 39.299 | 1.00 | 75.11 | N |
| ATOM | 2033 | CA | GLU | A | 132 | −34.622 | 80.796 | 39.802 | 1.00 | 76.59 | C |
| ATOM | 2035 | CB | GLU | A | 132 | −34.676 | 81.769 | 41.002 | 1.00 | 77.84 | C |
| ATOM | 2038 | CG | GLU | A | 132 | −33.434 | 82.630 | 41.266 | 1.00 | 87.06 | C |
| ATOM | 2041 | CD | GLU | A | 132 | −33.059 | 83.608 | 40.151 | 1.00 | 91.51 | C |
| ATOM | 2042 | OE1 | GLU | A | 132 | −33.316 | 83.354 | 38.948 | 1.00 | 80.75 | O |
| ATOM | 2043 | OE2 | GLU | A | 132 | −32.428 | 84.634 | 40.485 | 1.00 | 99.79 | O |
| ATOM | 2044 | C | GLU | A | 132 | −34.085 | 79.410 | 40.187 | 1.00 | 70.37 | C |
| ATOM | 2045 | O | GLU | A | 132 | −34.839 | 78.527 | 40.607 | 1.00 | 64.05 | O |
| ATOM | 2046 | N | TYR | A | 133 | −32.806 | 79.158 | 39.961 | 1.00 | 60.28 | N |
| ATOM | 2048 | CA | TYR | A | 133 | −32.318 | 77.817 | 40.247 | 1.00 | 59.48 | C |
| ATOM | 2050 | CB | TYR | A | 133 | −31.704 | 77.201 | 38.978 | 1.00 | 61.89 | C |
| ATOM | 2053 | CG | TYR | A | 133 | −32.709 | 76.866 | 37.884 | 1.00 | 62.29 | C |
| ATOM | 2054 | CD1 | TYR | A | 133 | −32.939 | 77.744 | 36.818 | 1.00 | 66.90 | C |
| ATOM | 2056 | CE1 | TYR | A | 133 | −33.890 | 77.452 | 35.833 | 1.00 | 51.14 | C |
| ATOM | 2058 | CZ | TYR | A | 133 | −34.603 | 76.263 | 35.903 | 1.00 | 52.82 | C |
| ATOM | 2059 | OH | TYR | A | 133 | −35.496 | 75.923 | 34.911 | 1.00 | 49.64 | O |
| ATOM | 2061 | CE2 | TYR | A | 133 | −34.409 | 75.393 | 36.963 | 1.00 | 44.59 | C |
| ATOM | 2063 | CD2 | TYR | A | 133 | −33.458 | 75.692 | 37.936 | 1.00 | 53.99 | C |
| ATOM | 2065 | C | TYR | A | 133 | −31.327 | 77.860 | 41.403 | 1.00 | 58.98 | C |
| ATOM | 2066 | O | TYR | A | 133 | −30.483 | 78.771 | 41.470 | 1.00 | 59.83 | O |
| ATOM | 2067 | N | SER | A | 134 | −31.422 | 76.887 | 42.314 | 1.00 | 57.86 | N |
| ATOM | 2069 | CA | SER | A | 134 | −30.434 | 76.758 | 43.407 | 1.00 | 59.72 | C |
| ATOM | 2071 | CB | SER | A | 134 | −30.652 | 75.507 | 44.304 | 1.00 | 68.63 | C |
| ATOM | 2074 | OG | SER | A | 134 | −31.978 | 74.930 | 44.370 | 1.00 | 58.41 | O |
| ATOM | 2076 | C | SER | A | 134 | −29.011 | 76.781 | 42.821 | 1.00 | 57.29 | C |
| ATOM | 2077 | O | SER | A | 134 | −28.219 | 77.657 | 43.138 | 1.00 | 60.88 | O |
| ATOM | 2078 | N | CYS | A | 135 | −28.750 | 75.945 | 41.821 | 1.00 | 58.64 | N |
| ATOM | 2080 | CA | CYS | A | 135 | −27.419 | 75.859 | 41.233 | 1.00 | 64.87 | C |
| ATOM | 2082 | CB | CYS | A | 135 | −26.890 | 74.438 | 41.345 | 1.00 | 68.31 | C |
| ATOM | 2085 | SG | CYS | A | 135 | −25.800 | 74.287 | 42.754 | 1.00 | 86.88 | S |
| ATOM | 2086 | C | CYS | A | 135 | −27.368 | 76.183 | 39.772 | 1.00 | 55.05 | C |
| ATOM | 2087 | O | CYS | A | 135 | −28.066 | 75.546 | 39.017 | 1.00 | 52.87 | O |
| ATOM | 2088 | N | VAL | A | 136 | −26.426 | 77.022 | 39.364 | 1.00 | 52.34 | N |
| ATOM | 2090 | CA | VAL | A | 136 | −26.117 | 77.141 | 37.953 | 1.00 | 52.88 | C |
| ATOM | 2092 | CB | VAL | A | 136 | −26.540 | 78.472 | 37.316 | 1.00 | 53.03 | C |
| ATOM | 2094 | CG1 | VAL | A | 136 | −26.035 | 78.472 | 35.888 | 1.00 | 65.89 | C |
| ATOM | 2098 | CG2 | VAL | A | 136 | −28.084 | 78.733 | 37.332 | 1.00 | 44.12 | C |
| ATOM | 2102 | C | VAL | A | 136 | −24.629 | 77.000 | 37.670 | 1.00 | 59.08 | C |
| ATOM | 2103 | O | VAL | A | 136 | −23.823 | 77.900 | 37.922 | 1.00 | 57.72 | O |
| ATOM | 2104 | N | VAL | A | 137 | −24.315 | 75.876 | 37.048 | 1.00 | 59.89 | N |
| ATOM | 2106 | CA | VAL | A | 137 | −22.959 | 75.478 | 36.736 | 1.00 | 60.99 | C |
| ATOM | 2108 | CB | VAL | A | 137 | −22.837 | 73.958 | 36.892 | 1.00 | 66.88 | C |
| ATOM | 2110 | CG1 | VAL | A | 137 | −21.427 | 73.483 | 36.625 | 1.00 | 58.41 | C |
| ATOM | 2114 | CG2 | VAL | A | 137 | −23.386 | 73.511 | 38.230 | 1.00 | 70.39 | C |
| ATOM | 2118 | C | VAL | A | 137 | −22.782 | 75.734 | 35.251 | 1.00 | 58.38 | C |
| ATOM | 2119 | O | VAL | A | 137 | −23.454 | 75.095 | 34.427 | 1.00 | 50.89 | O |
| ATOM | 2120 | N | LYS | A | 138 | −21.864 | 76.648 | 34.951 | 1.00 | 52.75 | N |
| ATOM | 2122 | CA | LYS | A | 138 | −21.374 | 76.910 | 33.614 | 1.00 | 49.24 | C |
| ATOM | 2124 | CB | LYS | A | 138 | −21.331 | 78.430 | 33.380 | 1.00 | 51.55 | C |
| ATOM | 2127 | CG | LYS | A | 138 | −20.259 | 78.956 | 32.396 | 1.00 | 69.85 | C |
| ATOM | 2130 | CD | LYS | A | 138 | −20.845 | 79.974 | 31.381 | 1.00 | 77.77 | C |
| ATOM | 2133 | CE | LYS | A | 138 | −19.804 | 80.528 | 30.379 | 1.00 | 80.34 | C |
| ATOM | 2136 | NZ | LYS | A | 138 | −18.389 | 80.213 | 30.762 | 1.00 | 74.78 | N |
| ATOM | 2140 | C | LYS | A | 138 | −19.991 | 76.267 | 33.464 | 1.00 | 48.29 | C |
| ATOM | 2141 | O | LYS | A | 138 | −19.036 | 76.655 | 34.095 | 1.00 | 45.43 | O |
| ATOM | 2142 | N | MET | A | 139 | −19.873 | 75.303 | 32.564 | 1.00 | 49.96 | N |
| ATOM | 2144 | CA | MET | A | 139 | −18.596 | 74.652 | 32.293 | 1.00 | 49.80 | C |
| ATOM | 2146 | CB | MET | A | 139 | −18.553 | 73.352 | 33.081 | 1.00 | 40.02 | C |
| ATOM | 2149 | CG | MET | A | 139 | −19.600 | 72.398 | 32.611 | 1.00 | 45.32 | C |
| ATOM | 2152 | SD | MET | A | 139 | −19.216 | 70.764 | 33.160 | 1.00 | 54.31 | S |
| ATOM | 2153 | CE | MET | A | 139 | −20.769 | 70.508 | 33.693 | 1.00 | 45.26 | C |
| ATOM | 2157 | C | MET | A | 139 | −18.346 | 74.374 | 30.788 | 1.00 | 50.47 | C |
| ATOM | 2158 | O | MET | A | 139 | −19.176 | 74.692 | 29.907 | 1.00 | 47.19 | O |
| ATOM | 2159 | N | PRO | A | 140 | −17.171 | 73.834 | 30.477 | 1.00 | 40.28 | N |
| ATOM | 2160 | CA | PRO | A | 140 | −16.837 | 73.555 | 29.083 | 1.00 | 39.07 | C |
| ATOM | 2162 | CB | PRO | A | 140 | −15.368 | 73.128 | 29.140 | 1.00 | 38.06 | C |
| ATOM | 2165 | CG | PRO | A | 140 | −14.861 | 73.620 | 30.422 | 1.00 | 36.22 | C |
| ATOM | 2168 | CD | PRO | A | 140 | −16.051 | 73.500 | 31.368 | 1.00 | 41.75 | C |
| ATOM | 2171 | C | PRO | A | 140 | −17.689 | 72.423 | 28.596 | 1.00 | 39.70 | C |
| ATOM | 2172 | O | PRO | A | 140 | −18.001 | 71.519 | 29.374 | 1.00 | 44.54 | O |
| ATOM | 2173 | N | SER | A | 141 | −18.086 | 72.487 | 27.333 | 1.00 | 43.07 | N |
| ATOM | 2175 | CA | SER | A | 141 | −19.075 | 71.531 | 26.825 | 1.00 | 45.99 | C |
| ATOM | 2177 | CB | SER | A | 141 | −19.806 | 72.097 | 25.602 | 1.00 | 47.95 | C |
| ATOM | 2180 | OG | SER | A | 141 | −18.922 | 72.335 | 24.520 | 1.00 | 44.30 | O |
| ATOM | 2182 | C | SER | A | 141 | −18.531 | 70.122 | 26.560 | 1.00 | 42.77 | C |
| ATOM | 2183 | O | SER | A | 141 | −19.207 | 69.123 | 26.821 | 1.00 | 41.24 | O |
| ATOM | 2184 | N | GLY | A | 142 | −17.301 | 70.065 | 26.058 | 1.00 | 49.13 | N |
| ATOM | 2186 | CA | GLY | A | 142 | −16.576 | 68.827 | 25.811 | 1.00 | 45.11 | C |
| ATOM | 2189 | C | GLY | A | 142 | −16.430 | 68.046 | 27.078 | 1.00 | 46.53 | C |
| ATOM | 2190 | O | GLY | A | 142 | −16.580 | 66.820 | 27.079 | 1.00 | 45.89 | O |
| ATOM | 2191 | N | GLU | A | 143 | −16.271 | 68.795 | 28.166 | 1.00 | 49.42 | N |
| ATOM | 2193 | CA | GLU | A | 143 | −16.001 | 68.208 | 29.468 | 1.00 | 52.86 | C |
| ATOM | 2195 | CB | GLU | A | 143 | −15.477 | 69.276 | 30.426 | 1.00 | 54.49 | C |
| ATOM | 2198 | CG | GLU | A | 143 | −14.761 | 68.706 | 31.634 | 1.00 | 73.36 | C |
| ATOM | 2201 | CD | GLU | A | 143 | −13.438 | 68.116 | 31.245 | 1.00 | 84.31 | C |
| ATOM | 2202 | OE1 | GLU | A | 143 | −12.590 | 68.925 | 30.800 | 1.00 | 91.64 | O |
| ATOM | 2203 | OE2 | GLU | A | 143 | −13.300 | 66.871 | 31.341 | 1.00 | 85.03 | O |
| ATOM | 2204 | C | GLU | A | 143 | −17.265 | 67.551 | 30.009 | 1.00 | 51.86 | C |
| ATOM | 2205 | O | GLU | A | 143 | −17.262 | 66.390 | 30.419 | 1.00 | 55.14 | O |
| ATOM | 2206 | N | PHE | A | 144 | −18.374 | 68.275 | 29.952 | 1.00 | 49.88 | N |
| ATOM | 2208 | CA | PHE | A | 144 | −19.627 | 67.697 | 30.394 | 1.00 | 40.14 | C |
| ATOM | 2210 | CB | PHE | A | 144 | −20.753 | 68.682 | 30.175 | 1.00 | 40.11 | C |
| ATOM | 2213 | CG | PHE | A | 144 | −22.076 | 68.215 | 30.721 | 1.00 | 33.58 | C |
| ATOM | 2214 | CD1 | PHE | A | 144 | −22.217 | 67.855 | 32.031 | 1.00 | 35.40 | C |
| ATOM | 2216 | CE1 | PHE | A | 144 | −23.402 | 67.449 | 32.503 | 1.00 | 33.63 | C |
| ATOM | 2218 | CZ | PHE | A | 144 | −24.458 | 67.394 | 31.677 | 1.00 | 32.05 | C |
| ATOM | 2220 | CE2 | PHE | A | 144 | −24.337 | 67.707 | 30.392 | 1.00 | 39.18 | C |
| ATOM | 2222 | CD2 | PHE | A | 144 | −23.162 | 68.130 | 29.909 | 1.00 | 27.38 | C |
| ATOM | 2224 | C | PHE | A | 144 | −19.928 | 66.415 | 29.636 | 1.00 | 38.47 | C |
| ATOM | 2225 | O | PHE | A | 144 | −20.529 | 65.486 | 30.190 | 1.00 | 31.05 | O |
| ATOM | 2226 | N | ALA | A | 145 | −19.545 | 66.368 | 28.366 | 1.00 | 37.47 | N |
| ATOM | 2228 | CA | ALA | A | 145 | −19.952 | 65.240 | 27.535 | 1.00 | 41.06 | C |
| ATOM | 2230 | CB | ALA | A | 145 | −19.919 | 65.619 | 26.073 | 1.00 | 43.67 | C |
| ATOM | 2234 | C | ALA | A | 145 | −18.989 | 64.095 | 27.808 | 1.00 | 45.57 | C |
| ATOM | 2235 | O | ALA | A | 145 | −19.365 | 62.907 | 27.737 | 1.00 | 34.53 | O |
| ATOM | 2236 | N | ARG | A | 146 | −17.745 | 64.458 | 28.125 | 1.00 | 42.15 | N |
| ATOM | 2238 | CA | ARG | A | 146 | −16.797 | 63.437 | 28.561 | 1.00 | 42.61 | C |
| ATOM | 2240 | CB | ARG | A | 146 | −15.412 | 64.054 | 28.746 | 1.00 | 41.32 | C |
| ATOM | 2243 | CG | ARG | A | 146 | −14.244 | 63.101 | 28.618 | 1.00 | 63.37 | C |
| ATOM | 2246 | CD | ARG | A | 146 | −12.852 | 63.778 | 28.765 | 1.00 | 92.47 | C |
| ATOM | 2249 | NE | ARG | A | 146 | −11.760 | 63.062 | 28.087 | 1.00 | 107.57 | N |
| ATOM | 2251 | CZ | ARG | A | 146 | −11.598 | 61.731 | 28.043 | 1.00 | 105.37 | C |
| ATOM | 2252 | NH1 | ARG | A | 146 | −12.472 | 60.902 | 28.620 | 1.00 | 98.78 | N |
| ATOM | 2255 | NH2 | ARG | A | 146 | −10.545 | 61.219 | 27.404 | 1.00 | 92.98 | N |
| ATOM | 2258 | C | ARG | A | 146 | −17.332 | 62.805 | 29.847 | 1.00 | 38.03 | C |
| ATOM | 2259 | O | ARG | A | 146 | −17.375 | 61.587 | 29.979 | 1.00 | 40.15 | O |
| ATOM | 2260 | N | ILE | A | 147 | −17.832 | 63.628 | 30.762 | 1.00 | 30.91 | N |
| ATOM | 2262 | CA | ILE | A | 147 | −18.278 | 63.122 | 32.053 | 1.00 | 35.65 | C |
| ATOM | 2264 | CB | ILE | A | 147 | −18.570 | 64.287 | 33.009 | 1.00 | 31.97 | C |
| ATOM | 2266 | CG1 | ILE | A | 147 | −17.266 | 64.710 | 33.675 | 1.00 | 36.26 | C |
| ATOM | 2269 | CD1 | ILE | A | 147 | −17.166 | 66.190 | 33.965 | 1.00 | 39.37 | C |
| ATOM | 2273 | CG2 | ILE | A | 147 | −19.581 | 63.911 | 34.061 | 1.00 | 34.50 | C |
| ATOM | 2277 | C | ILE | A | 147 | −19.487 | 62.226 | 31.902 | 1.00 | 35.99 | C |
| ATOM | 2278 | O | ILE | A | 147 | −19.580 | 61.158 | 32.508 | 1.00 | 39.03 | O |
| ATOM | 2279 | N | CYS | A | 148 | −20.422 | 62.671 | 31.078 | 1.00 | 40.34 | N |
| ATOM | 2281 | CA | CYS | A | 148 | −21.608 | 61.866 | 30.829 | 1.00 | 43.12 | C |
| ATOM | 2283 | CB | CYS | A | 148 | −22.686 | 62.651 | 30.085 | 1.00 | 35.79 | C |
| ATOM | 2286 | SG | CYS | A | 148 | −23.347 | 63.993 | 31.106 | 1.00 | 41.14 | S |
| ATOM | 2287 | C | CYS | A | 148 | −21.280 | 60.539 | 30.147 | 1.00 | 46.01 | C |
| ATOM | 2288 | O | CYS | A | 148 | −21.922 | 59.522 | 30.430 | 1.00 | 49.60 | O |
| ATOM | 2289 | N | ARG | A | 149 | −20.268 | 60.521 | 29.287 | 1.00 | 47.95 | N |
| ATOM | 2291 | CA | ARG | A | 149 | −19.997 | 59.325 | 28.495 | 1.00 | 51.34 | C |
| ATOM | 2293 | CB | ARG | A | 149 | −19.201 | 59.688 | 27.250 | 1.00 | 51.91 | C |
| ATOM | 2296 | CG | ARG | A | 149 | −19.087 | 58.585 | 26.241 | 1.00 | 53.94 | C |
| ATOM | 2299 | CD | ARG | A | 149 | −17.653 | 58.362 | 25.793 | 1.00 | 80.90 | C |
| ATOM | 2302 | NE | ARG | A | 149 | −16.841 | 59.582 | 25.837 | 1.00 | 101.89 | N |
| ATOM | 2304 | CZ | ARG | A | 149 | −15.504 | 59.594 | 25.835 | 1.00 | 113.49 | C |
| ATOM | 2305 | NH1 | ARG | A | 149 | −14.828 | 58.448 | 25.777 | 1.00 | 119.42 | N |
| ATOM | 2308 | NH2 | ARG | A | 149 | −14.835 | 60.745 | 25.873 | 1.00 | 92.82 | N |
| ATOM | 2311 | C | ARG | A | 149 | −19.214 | 58.317 | 29.328 | 1.00 | 55.45 | C |
| ATOM | 2312 | O | ARG | A | 149 | −19.491 | 57.112 | 29.263 | 1.00 | 58.72 | O |
| ATOM | 2313 | N | ASP | A | 150 | −18.271 | 58.816 | 30.130 | 1.00 | 55.28 | N |
| ATOM | 2315 | CA | ASP | A | 150 | −17.482 | 57.952 | 31.012 | 1.00 | 49.14 | C |
| ATOM | 2317 | CB | ASP | A | 150 | −16.373 | 58.706 | 31.719 | 1.00 | 45.70 | C |
| ATOM | 2320 | CG | ASP | A | 150 | −15.248 | 59.098 | 30.777 | 1.00 | 37.08 | C |
| ATOM | 2321 | OD1 | ASP | A | 150 | −15.308 | 58.807 | 29.569 | 1.00 | 49.75 | O |
| ATOM | 2322 | OD2 | ASP | A | 150 | −14.295 | 59.801 | 31.138 | 1.00 | 45.86 | O |
| ATOM | 2323 | C | ASP | A | 150 | −18.350 | 57.303 | 32.046 | 1.00 | 43.74 | C |
| ATOM | 2324 | O | ASP | A | 150 | −18.347 | 56.093 | 32.132 | 1.00 | 51.65 | O |
| ATOM | 2325 | N | LEU | A | 151 | −19.114 | 58.086 | 32.797 | 1.00 | 43.39 | N |
| ATOM | 2327 | CA | LEU | A | 151 | −19.923 | 57.540 | 33.883 | 1.00 | 42.58 | C |
| ATOM | 2329 | CB | LEU | A | 151 | −20.588 | 58.671 | 34.670 | 1.00 | 45.65 | C |
| ATOM | 2332 | CG | LEU | A | 151 | −19.694 | 59.527 | 35.575 | 1.00 | 47.20 | C |
| ATOM | 2334 | CD1 | LEU | A | 151 | −20.361 | 60.748 | 36.257 | 1.00 | 38.21 | C |
| ATOM | 2338 | CD2 | LEU | A | 151 | −19.130 | 58.614 | 36.621 | 1.00 | 58.27 | C |
| ATOM | 2342 | C | LEU | A | 151 | −20.992 | 56.571 | 33.376 | 1.00 | 44.49 | C |
| ATOM | 2343 | O | LEU | A | 151 | −21.598 | 55.838 | 34.154 | 1.00 | 48.34 | O |
| ATOM | 2344 | N | SER | A | 152 | −21.246 | 56.579 | 32.074 | 1.00 | 48.78 | N |
| ATOM | 2346 | CA | SER | A | 152 | −22.183 | 55.627 | 31.466 | 1.00 | 52.92 | C |
| ATOM | 2348 | CB | SER | A | 152 | −22.288 | 55.830 | 29.951 | 1.00 | 49.22 | C |
| ATOM | 2351 | OG | SER | A | 152 | −22.891 | 57.068 | 29.664 | 1.00 | 63.43 | O |
| ATOM | 2353 | C | SER | A | 152 | −21.686 | 54.206 | 31.648 | 1.00 | 52.98 | C |
| ATOM | 2354 | O | SER | A | 152 | −22.484 | 53.277 | 31.508 | 1.00 | 60.88 | O |
| ATOM | 2355 | N | HIS | A | 153 | −20.369 | 54.063 | 31.819 | 1.00 | 48.85 | N |
| ATOM | 2357 | CA | HIS | A | 153 | −19.674 | 52.789 | 31.690 | 1.00 | 54.98 | C |
| ATOM | 2359 | CB | HIS | A | 153 | −18.210 | 52.944 | 31.269 | 1.00 | 61.05 | C |
| ATOM | 2362 | CG | HIS | A | 153 | −18.003 | 53.384 | 29.851 | 1.00 | 69.69 | C |
| ATOM | 2363 | ND1 | HIS | A | 153 | −18.722 | 52.874 | 28.786 | 1.00 | 70.26 | N |
| ATOM | 2365 | CE1 | HIS | A | 153 | −18.346 | 53.486 | 27.679 | 1.00 | 74.70 | C |
| ATOM | 2367 | NE2 | HIS | A | 153 | −17.406 | 54.364 | 27.988 | 1.00 | 92.71 | N |
| ATOM | 2369 | CD2 | HIS | A | 153 | −17.143 | 54.294 | 29.335 | 1.00 | 56.46 | C |
| ATOM | 2371 | C | HIS | A | 153 | −19.652 | 52.226 | 33.093 | 1.00 | 58.55 | C |
| ATOM | 2372 | O | HIS | A | 153 | −19.515 | 51.009 | 33.303 | 1.00 | 56.25 | O |
| ATOM | 2373 | N | ILE | A | 154 | −19.840 | 53.132 | 34.045 | 1.00 | 53.80 | N |
| ATOM | 2375 | CA | ILE | A | 154 | −20.059 | 52.719 | 35.420 | 1.00 | 55.79 | C |
| ATOM | 2377 | CB | ILE | A | 154 | −19.738 | 53.850 | 36.366 | 1.00 | 58.71 | C |
| ATOM | 2379 | CG1 | ILE | A | 154 | −18.384 | 54.465 | 36.013 | 1.00 | 52.00 | C |
| ATOM | 2382 | CD1 | ILE | A | 154 | −17.274 | 53.522 | 36.238 | 1.00 | 60.24 | C |
| ATOM | 2386 | CG2 | ILE | A | 154 | −19.792 | 53.300 | 37.768 | 1.00 | 61.56 | C |
| ATOM | 2390 | C | ILE | A | 154 | −21.464 | 52.251 | 35.753 | 1.00 | 53.48 | C |
| ATOM | 2391 | O | ILE | A | 154 | −21.602 | 51.266 | 36.470 | 1.00 | 52.81 | O |
| ATOM | 2392 | N | GLY | A | 155 | −22.476 | 52.980 | 35.276 | 1.00 | 58.29 | N |
| ATOM | 2394 | CA | GLY | A | 155 | −23.840 | 52.949 | 35.806 | 1.00 | 57.25 | C |
| ATOM | 2397 | C | GLY | A | 155 | −24.833 | 53.567 | 34.818 | 1.00 | 55.07 | C |
| ATOM | 2398 | O | GLY | A | 155 | −24.442 | 53.982 | 33.742 | 1.00 | 51.97 | O |
| ATOM | 2399 | N | ASP | A | 156 | −26.114 | 53.623 | 35.177 | 1.00 | 53.05 | N |
| ATOM | 2401 | CA | ASP | A | 156 | −27.188 | 54.087 | 34.309 | 1.00 | 49.04 | C |
| ATOM | 2403 | CB | ASP | A | 156 | −28.358 | 53.122 | 34.391 | 1.00 | 49.98 | C |
| ATOM | 2406 | CG | ASP | A | 156 | −27.934 | 51.705 | 34.174 | 1.00 | 67.25 | C |
| ATOM | 2407 | OD1 | ASP | A | 156 | −27.006 | 51.458 | 33.364 | 1.00 | 89.24 | O |
| ATOM | 2408 | OD2 | ASP | A | 156 | −28.457 | 50.775 | 34.817 | 1.00 | 99.03 | O |
| ATOM | 2409 | C | ASP | A | 156 | −27.719 | 55.443 | 34.700 | 1.00 | 45.96 | C |
| ATOM | 2410 | O | ASP | A | 156 | −28.446 | 56.105 | 33.960 | 1.00 | 54.91 | O |
| ATOM | 2411 | N | ALA | A | 157 | −27.349 | 55.826 | 35.900 | 1.00 | 50.80 | N |
| ATOM | 2413 | CA | ALA | A | 157 | −27.854 | 57.014 | 36.534 | 1.00 | 56.16 | C |
| ATOM | 2415 | CB | ALA | A | 157 | −28.719 | 56.609 | 37.730 | 1.00 | 64.47 | C |
| ATOM | 2419 | C | ALA | A | 157 | −26.674 | 57.809 | 37.021 | 1.00 | 47.69 | C |
| ATOM | 2420 | O | ALA | A | 157 | −25.688 | 57.263 | 37.484 | 1.00 | 51.27 | O |
| ATOM | 2421 | N | VAL | A | 158 | −26.819 | 59.116 | 37.022 | 1.00 | 50.87 | N |
| ATOM | 2423 | CA | VAL | A | 158 | −25.792 | 59.934 | 37.645 | 1.00 | 51.48 | C |
| ATOM | 2425 | CB | VAL | A | 158 | −25.104 | 60.896 | 36.662 | 1.00 | 45.88 | C |
| ATOM | 2427 | CG1 | VAL | A | 158 | −26.136 | 61.781 | 35.963 | 1.00 | 45.77 | C |
| ATOM | 2431 | CG2 | VAL | A | 158 | −24.125 | 61.773 | 37.393 | 1.00 | 54.02 | C |
| ATOM | 2435 | C | VAL | A | 158 | −26.423 | 60.814 | 38.684 | 1.00 | 54.04 | C |
| ATOM | 2436 | O | VAL | A | 158 | −27.522 | 61.350 | 38.516 | 1.00 | 50.48 | O |
| ATOM | 2437 | N | VAL | A | 159 | −25.649 | 61.024 | 39.727 | 1.00 | 53.18 | N |
| ATOM | 2439 | CA | VAL | A | 159 | −26.092 | 61.889 | 40.770 | 1.00 | 56.90 | C |
| ATOM | 2441 | CB | VAL | A | 159 | −25.958 | 61.204 | 42.138 | 1.00 | 62.82 | C |
| ATOM | 2443 | CG1 | VAL | A | 159 | −26.185 | 62.236 | 43.269 | 1.00 | 65.52 | C |
| ATOM | 2447 | CG2 | VAL | A | 159 | −26.916 | 60.008 | 42.220 | 1.00 | 60.24 | C |
| ATOM | 2451 | C | VAL | A | 159 | −25.203 | 63.089 | 40.735 | 1.00 | 52.40 | C |
| ATOM | 2452 | O | VAL | A | 159 | −24.001 | 62.987 | 40.923 | 1.00 | 61.61 | O |
| ATOM | 2453 | N | ILE | A | 160 | −25.824 | 64.243 | 40.570 | 1.00 | 56.99 | N |
| ATOM | 2455 | CA | ILE | A | 160 | −25.090 | 65.493 | 40.430 | 1.00 | 53.91 | C |
| ATOM | 2457 | CB | ILE | A | 160 | −25.670 | 66.289 | 39.264 | 1.00 | 49.43 | C |
| ATOM | 2459 | CG1 | ILE | A | 160 | −25.830 | 65.391 | 38.029 | 1.00 | 35.23 | C |
| ATOM | 2462 | CD1 | ILE | A | 160 | −26.295 | 66.165 | 36.884 | 1.00 | 50.63 | C |
| ATOM | 2466 | CG2 | ILE | A | 160 | −24.766 | 67.467 | 38.979 | 1.00 | 42.07 | C |
| ATOM | 2470 | C | ILE | A | 160 | −25.303 | 66.291 | 41.672 | 1.00 | 56.36 | C |
| ATOM | 2471 | O | ILE | A | 160 | −26.457 | 66.437 | 42.077 | 1.00 | 58.69 | O |
| ATOM | 2472 | N | SER | A | 161 | −24.213 | 66.794 | 42.246 | 1.00 | 60.54 | N |
| ATOM | 2474 | CA | SER | A | 161 | −24.238 | 67.525 | 43.515 | 1.00 | 61.07 | C |
| ATOM | 2476 | CB | SER | A | 161 | −23.728 | 66.692 | 44.701 | 1.00 | 56.97 | C |
| ATOM | 2479 | OG | SER | A | 161 | −23.697 | 65.318 | 44.416 | 1.00 | 72.05 | O |
| ATOM | 2481 | C | SER | A | 161 | −23.356 | 68.759 | 43.447 | 1.00 | 75.52 | C |
| ATOM | 2482 | O | SER | A | 161 | −22.211 | 68.733 | 42.955 | 1.00 | 76.19 | O |
| ATOM | 2483 | N | CYS | A | 162 | −23.861 | 69.807 | 44.090 | 1.00 | 85.72 | N |
| ATOM | 2485 | CA | CYS | A | 162 | −23.372 | 71.158 | 43.888 | 1.00 | 84.99 | C |
| ATOM | 2487 | CB | CYS | A | 162 | −24.320 | 71.905 | 42.961 | 1.00 | 86.27 | C |
| ATOM | 2490 | SG | CYS | A | 162 | −23.435 | 72.825 | 41.695 | 1.00 | 87.36 | S |
| ATOM | 2491 | C | CYS | A | 162 | −23.321 | 71.847 | 45.228 | 1.00 | 90.35 | C |
| ATOM | 2492 | O | CYS | A | 162 | −24.252 | 71.732 | 46.026 | 1.00 | 92.80 | O |
| ATOM | 2493 | N | ALA | A | 163 | −22.198 | 72.512 | 45.472 | 1.00 | 99.06 | N |
| ATOM | 2495 | CA | ALA | A | 163 | −22.056 | 73.498 | 46.540 | 1.00 | 102.05 | C |
| ATOM | 2497 | CB | ALA | A | 163 | −21.475 | 72.833 | 47.801 | 1.00 | 96.72 | C |
| ATOM | 2501 | C | ALA | A | 163 | −21.160 | 74.644 | 46.027 | 1.00 | 106.93 | C |
| ATOM | 2502 | O | ALA | A | 163 | −20.834 | 74.707 | 44.839 | 1.00 | 104.16 | O |
| ATOM | 2503 | N | LYS | A | 164 | −20.780 | 75.565 | 46.910 | 1.00 | 112.28 | N |
| ATOM | 2505 | CA | LYS | A | 164 | −20.235 | 76.865 | 46.503 | 1.00 | 113.58 | C |
| ATOM | 2507 | CB | LYS | A | 164 | −20.158 | 77.775 | 47.749 | 1.00 | 114.59 | C |
| ATOM | 2510 | CG | LYS | A | 164 | −19.205 | 78.973 | 47.691 | 1.00 | 119.56 | C |
| ATOM | 2513 | CD | LYS | A | 164 | −19.773 | 80.155 | 46.912 | 1.00 | 119.19 | C |
| ATOM | 2516 | CE | LYS | A | 164 | −18.691 | 80.804 | 46.043 | 1.00 | 118.93 | C |
| ATOM | 2519 | NZ | LYS | A | 164 | −19.118 | 80.978 | 44.620 | 1.00 | 116.90 | N |
| ATOM | 2523 | C | LYS | A | 164 | −18.887 | 76.734 | 45.750 | 1.00 | 111.07 | C |
| ATOM | 2524 | O | LYS | A | 164 | −18.488 | 77.604 | 44.967 | 1.00 | 109.47 | O |
| ATOM | 2525 | N | ASP | A | 165 | −18.213 | 75.604 | 45.935 | 1.00 | 109.60 | N |
| ATOM | 2527 | CA | ASP | A | 165 | −16.798 | 75.471 | 45.582 | 1.00 | 108.17 | C |
| ATOM | 2529 | CB | ASP | A | 165 | −15.996 | 74.977 | 46.809 | 1.00 | 109.43 | C |
| ATOM | 2532 | CG | ASP | A | 165 | −16.644 | 73.770 | 47.503 | 1.00 | 112.52 | C |
| ATOM | 2533 | OD1 | ASP | A | 165 | −16.007 | 72.697 | 47.582 | 1.00 | 109.83 | O |
| ATOM | 2534 | OD2 | ASP | A | 165 | −17.798 | 73.781 | 47.988 | 1.00 | 118.27 | O |
| ATOM | 2535 | C | ASP | A | 165 | −16.597 | 74.545 | 44.354 | 1.00 | 101.95 | C |
| ATOM | 2536 | O | ASP | A | 165 | −15.599 | 74.656 | 43.630 | 1.00 | 96.39 | O |
| ATOM | 2537 | N | GLY | A | 166 | −17.546 | 73.632 | 44.125 | 1.00 | 92.07 | N |
| ATOM | 2539 | CA | GLY | A | 166 | −17.558 | 72.814 | 42.921 | 1.00 | 81.89 | C |
| ATOM | 2542 | C | GLY | A | 166 | −18.760 | 71.895 | 42.740 | 1.00 | 70.09 | C |
| ATOM | 2543 | O | GLY | A | 166 | −19.683 | 71.867 | 43.552 | 1.00 | 69.71 | O |
| ATOM | 2544 | N | VAL | A | 167 | −18.743 | 71.122 | 41.663 | 1.00 | 57.49 | N |
| ATOM | 2646 | CA | VAL | A | 167 | −19.780 | 70.128 | 41.407 | 1.00 | 50.09 | C |
| ATOM | 2548 | CB | VAL | A | 167 | −20.583 | 70.465 | 40.123 | 1.00 | 45.31 | C |
| ATOM | 2550 | CG1 | VAL | A | 167 | −19.677 | 70.818 | 38.946 | 1.00 | 48.65 | C |
| ATOM | 2554 | CG2 | VAL | A | 167 | −21.496 | 69.317 | 39.736 | 1.00 | 51.87 | C |
| ATOM | 2558 | C | VAL | A | 167 | −19.173 | 68.733 | 41.264 | 1.00 | 50.85 | C |
| ATOM | 2559 | O | VAL | A | 167 | −17.999 | 68.581 | 40.940 | 1.00 | 39.95 | O |
| ATOM | 2560 | N | LYS | A | 168 | −20.000 | 67.719 | 41.475 | 1.00 | 53.28 | N |
| ATOM | 2562 | CA | LYS | A | 168 | −19.540 | 66.348 | 41.482 | 1.00 | 60.80 | C |
| ATOM | 2564 | CB | LYS | A | 168 | −19.294 | 65.908 | 42.934 | 1.00 | 66.21 | C |
| ATOM | 2567 | CG | LYS | A | 168 | −19.692 | 64.469 | 43.305 | 1.00 | 67.01 | C |
| ATOM | 2570 | CD | LYS | A | 168 | −18.625 | 63.782 | 44.203 | 1.00 | 58.02 | C |
| ATOM | 2573 | CE | LYS | A | 168 | −18.810 | 64.067 | 45.701 | 1.00 | 68.01 | C |
| ATOM | 2576 | NZ | LYS | A | 168 | −19.457 | 62.933 | 46.461 | 1.00 | 67.91 | N |
| ATOM | 2580 | C | LYS | A | 168 | −20.578 | 65.461 | 40.801 | 1.00 | 65.92 | C |
| ATOM | 2581 | O | LYS | A | 168 | −21.788 | 65.517 | 41.064 | 1.00 | 67.90 | O |
| ATOM | 2582 | N | PHE | A | 169 | −20.092 | 64.597 | 39.928 | 1.00 | 63.81 | N |
| ATOM | 2584 | CA | PHE | A | 169 | −20.975 | 63.611 | 39.328 | 1.00 | 60.26 | C |
| ATOM | 2586 | CB | PHE | A | 169 | −20.878 | 63.758 | 37.801 | 1.00 | 63.33 | C |
| ATOM | 2589 | CG | PHE | A | 169 | −20.932 | 65.192 | 37.316 | 1.00 | 53.28 | C |
| ATOM | 2690 | CD1 | PHE | A | 169 | −19.807 | 66.004 | 37.340 | 1.00 | 55.49 | C |
| ATOM | 2592 | CE1 | PHE | A | 169 | −19.844 | 67.314 | 36.868 | 1.00 | 54.02 | C |
| ATOM | 2594 | CZ | PHE | A | 169 | −21.020 | 67.843 | 36.404 | 1.00 | 54.21 | C |
| ATOM | 2596 | CE2 | PHE | A | 169 | −22.160 | 67.032 | 36.360 | 1.00 | 64.60 | C |
| ATOM | 2598 | CD2 | PHE | A | 169 | −22.110 | 65.717 | 36.809 | 1.00 | 53.33 | C |
| ATOM | 2600 | C | PHE | A | 169 | −20.619 | 62.182 | 39.824 | 1.00 | 56.94 | C |
| ATOM | 2601 | O | PHE | A | 169 | −19.445 | 61.804 | 39.871 | 1.00 | 55.67 | O |
| ATOM | 2602 | N | SER | A | 170 | −21.620 | 61.398 | 40.217 | 1.00 | 43.36 | N |
| ATOM | 2604 | CA | SER | A | 170 | −21.367 | 60.110 | 40.819 | 1.00 | 45.85 | C |
| ATOM | 2606 | CB | SER | A | 170 | −21.780 | 60.060 | 42.289 | 1.00 | 49.84 | C |
| ATOM | 2609 | OG | SER | A | 170 | −21.003 | 60.923 | 43.090 | 1.00 | 53.55 | O |
| ATOM | 2611 | C | SER | A | 170 | −22.207 | 59.105 | 40.101 | 1.00 | 49.23 | C |
| ATOM | 2612 | O | SER | A | 170 | −23.262 | 59.458 | 39.599 | 1.00 | 58.11 | O |
| ATOM | 2613 | N | ALA | A | 171 | −21.777 | 57.847 | 40.149 | 1.00 | 54.22 | N |
| ATOM | 2615 | CA | ALA | A | 171 | −22.511 | 56.733 | 39.564 | 1.00 | 50.90 | C |
| ATOM | 2617 | CB | ALA | A | 171 | −22.334 | 56.784 | 38.060 | 1.00 | 55.90 | C |
| ATOM | 2621 | C | ALA | A | 171 | −22.046 | 55.371 | 40.123 | 1.00 | 52.49 | C |
| ATOM | 2622 | O | ALA | A | 171 | −20.940 | 55.261 | 40.661 | 1.00 | 54.85 | O |
| ATOM | 2623 | N | SER | A | 172 | −22.881 | 54.339 | 39.971 | 1.00 | 50.65 | N |
| ATOM | 2625 | CA | SER | A | 172 | −22.683 | 53.014 | 40.570 | 1.00 | 47.64 | C |
| ATOM | 2627 | CB | SER | A | 172 | −23.440 | 52.945 | 41.878 | 1.00 | 44.64 | C |
| ATOM | 2630 | OG | SER | A | 172 | −22.861 | 53.915 | 42.735 | 1.00 | 73.15 | O |
| ATOM | 2632 | C | SER | A | 172 | −23.234 | 51.913 | 39.698 | 1.00 | 46.65 | C |
| ATOM | 2633 | O | SER | A | 172 | −24.040 | 52.180 | 38.815 | 1.00 | 58.18 | O |
| ATOM | 2634 | N | GLY | A | 173 | −22.803 | 50.675 | 39.928 | 1.00 | 47.06 | N |
| ATOM | 2636 | CA | GLY | A | 173 | −23.078 | 49.577 | 39.012 | 1.00 | 39.97 | C |
| ATOM | 2639 | C | GLY | A | 173 | −22.349 | 48.310 | 39.429 | 1.00 | 48.64 | C |
| ATOM | 2640 | O | GLY | A | 173 | −21.628 | 48.290 | 40.422 | 1.00 | 57.24 | O |
| ATOM | 2641 | N | GLU | A | 174 | −22.557 | 47.236 | 38.679 | 1.00 | 50.93 | N |
| ATOM | 2643 | CA | GLU | A | 174 | −21.815 | 46.001 | 38.835 | 1.00 | 55.79 | C |
| ATOM | 2645 | CB | GLU | A | 174 | −21.952 | 45.173 | 37.558 | 1.00 | 64.86 | C |
| ATOM | 2648 | CG | GLU | A | 174 | −23.214 | 44.309 | 37.484 | 1.00 | 85.96 | C |
| ATOM | 2651 | CD | GLU | A | 174 | −23.390 | 43.570 | 36.151 | 1.00 | 100.89 | C |
| ATOM | 2652 | OE1 | GLU | A | 174 | −23.795 | 42.380 | 36.178 | 1.00 | 106.18 | O |
| ATOM | 2653 | OE2 | GLU | A | 174 | −23.138 | 44.167 | 35.074 | 1.00 | 81.12 | O |
| ATOM | 2654 | C | GLU | A | 174 | −20.332 | 46.244 | 39.065 | 1.00 | 63.88 | C |
| ATOM | 2655 | O | GLU | A | 174 | −19.765 | 45.632 | 39.973 | 1.00 | 63.15 | O |
| ATOM | 2656 | N | LEU | A | 175 | −19.713 | 47.104 | 38.239 | 1.00 | 68.97 | N |
| ATOM | 2658 | CA | LEU | A | 175 | −18.253 | 47.328 | 38.246 | 1.00 | 63.55 | C |
| ATOM | 2660 | CB | LEU | A | 175 | −17.798 | 48.386 | 37.235 | 1.00 | 61.15 | C |
| ATOM | 2663 | CG | LEU | A | 175 | −18.140 | 48.078 | 35.780 | 1.00 | 68.89 | C |
| ATOM | 2665 | CD1 | LEU | A | 175 | −17.100 | 48.737 | 34.880 | 1.00 | 54.94 | C |
| ATOM | 2669 | CD2 | LEU | A | 175 | −18.344 | 46.567 | 35.447 | 1.00 | 55.58 | C |
| ATOM | 2673 | C | LEU | A | 175 | −17.756 | 47.799 | 39.597 | 1.00 | 60.19 | C |
| ATOM | 2674 | O | LEU | A | 175 | −16.651 | 47.454 | 40.006 | 1.00 | 65.70 | O |
| ATOM | 2675 | N | GLY | A | 176 | −18.518 | 48.662 | 40.249 | 1.00 | 50.49 | N |
| ATOM | 2677 | CA | GLY | A | 176 | −17.888 | 49.546 | 41.193 | 1.00 | 51.15 | C |
| ATOM | 2680 | C | GLY | A | 176 | −18.641 | 50.842 | 41.200 | 1.00 | 53.43 | C |
| ATOM | 2681 | O | GLY | A | 176 | −19.816 | 50.860 | 40.803 | 1.00 | 60.16 | O |
| ATOM | 2682 | N | ASN | A | 177 | −17.985 | 51.888 | 41.699 | 1.00 | 46.58 | N |
| ATOM | 2684 | CA | ASN | A | 177 | −18.552 | 53.229 | 41.676 | 1.00 | 50.59 | C |
| ATOM | 2686 | CB | ASN | A | 177 | −19.215 | 53.550 | 43.008 | 1.00 | 50.92 | C |
| ATOM | 2689 | CG | ASN | A | 177 | −18.200 | 53.706 | 44.105 | 1.00 | 69.14 | C |
| ATOM | 2690 | OD1 | ASN | A | 177 | −17.598 | 54.789 | 44.281 | 1.00 | 64.72 | O |
| ATOM | 2691 | ND2 | ASN | A | 177 | −17.921 | 52.591 | 44.785 | 1.00 | 63.30 | N |
| ATOM | 2694 | C | ASN | A | 177 | −17.499 | 54.278 | 41.375 | 1.00 | 47.32 | C |
| ATOM | 2695 | O | ASN | A | 177 | −16.304 | 54.010 | 41.305 | 1.00 | 52.04 | O |
| ATOM | 2696 | N | GLY | A | 178 | −17.957 | 55.488 | 41.132 | 1.00 | 48.03 | N |
| ATOM | 2698 | CA | GLY | A | 178 | −17.048 | 56.538 | 40.718 | 1.00 | 49.16 | C |
| ATOM | 2701 | C | GLY | A | 178 | −17.656 | 57.914 | 40.911 | 1.00 | 44.66 | C |
| ATOM | 2702 | O | GLY | A | 178 | −18.871 | 58.100 | 40.876 | 1.00 | 43.72 | O |
| ATOM | 2703 | N | ASN | A | 179 | −16.775 | 58.880 | 41.095 | 1.00 | 42.11 | N |
| ATOM | 2705 | CA | ASN | A | 179 | −17.147 | 60.238 | 41.401 | 1.00 | 50.23 | C |
| ATOM | 2707 | CB | ASN | A | 179 | −17.004 | 60.479 | 42.913 | 1.00 | 52.46 | C |
| ATOM | 2710 | CG | ASN | A | 179 | −17.935 | 59.607 | 43.742 | 1.00 | 73.19 | C |
| ATOM | 2711 | OD1 | ASN | A | 179 | −18.195 | 58.436 | 43.426 | 1.00 | 85.81 | O |
| ATOM | 2712 | ND2 | ASN | A | 179 | −18.446 | 60.184 | 44.827 | 1.00 | 99.53 | N |
| ATOM | 2715 | C | ASN | A | 179 | −16.128 | 61.070 | 40.621 | 1.00 | 57.10 | C |
| ATOM | 2716 | O | ASN | A | 179 | −14.928 | 60.751 | 40.628 | 1.00 | 60.50 | O |
| ATOM | 2717 | N | ILE | A | 180 | −16.607 | 62.103 | 39.929 | 1.00 | 59.33 | N |
| ATOM | 2719 | CA | ILE | A | 180 | −15.753 | 63.098 | 39.273 | 1.00 | 55.85 | C |
| ATOM | 2721 | CB | ILE | A | 180 | −16.124 | 63.133 | 37.782 | 1.00 | 53.70 | C |
| ATOM | 2723 | CG1 | ILE | A | 180 | −16.300 | 61.672 | 37.312 | 1.00 | 63.42 | C |
| ATOM | 2726 | CD1 | ILE | A | 180 | −16.502 | 61.400 | 35.818 | 1.00 | 54.35 | C |
| ATOM | 2730 | CG2 | ILE | A | 180 | −15.098 | 63.942 | 36.993 | 1.00 | 50.34 | C |
| ATOM | 2734 | C | ILE | A | 180 | −16.010 | 64.447 | 39.945 | 1.00 | 60.34 | C |
| ATOM | 2735 | O | ILE | A | 180 | −17.168 | 64.805 | 40.219 | 1.00 | 61.70 | O |
| ATOM | 2736 | N | LYS | A | 181 | −14.934 | 65.148 | 40.304 | 1.00 | 60.17 | N |
| ATOM | 2738 | CA | LYS | A | 181 | −15.057 | 66.388 | 41.067 | 1.00 | 56.85 | C |
| ATOM | 2740 | CB | LYS | A | 181 | −14.381 | 66.340 | 42.453 | 1.00 | 58.37 | C |
| ATOM | 2743 | CG | LYS | A | 181 | −15.320 | 65.918 | 43.624 | 1.00 | 73.40 | C |
| ATOM | 2746 | CD | LYS | A | 181 | −14.734 | 66.107 | 45.055 | 1.00 | 96.92 | C |
| ATOM | 2749 | CE | LYS | A | 181 | −15.146 | 67.444 | 45.730 | 1.00 | 106.19 | C |
| ATOM | 2752 | NZ | LYS | A | 181 | −15.828 | 68.441 | 44.812 | 1.00 | 99.99 | N |
| ATOM | 2756 | C | LYS | A | 181 | −14.440 | 67.474 | 40.228 | 1.00 | 57.37 | C |
| ATOM | 2757 | O | LYS | A | 181 | −13.287 | 67.386 | 39.814 | 1.00 | 59.74 | O |
| ATOM | 2758 | N | LEU | A | 182 | −15.257 | 68.488 | 39.983 | 1.00 | 60.39 | N |
| ATOM | 2760 | CA | LEU | A | 182 | −14.934 | 69.605 | 39.114 | 1.00 | 64.36 | C |
| ATOM | 2762 | CB | LEU | A | 182 | −16.059 | 69.824 | 38.078 | 1.00 | 65.64 | C |
| ATOM | 2765 | CG | LEU | A | 182 | −16.099 | 69.035 | 36.758 | 1.00 | 62.89 | C |
| ATOM | 2767 | CD1 | LEU | A | 182 | −17.200 | 69.555 | 35.803 | 1.00 | 75.00 | C |
| ATOM | 2771 | CD2 | LEU | A | 182 | −14.759 | 69.027 | 36.038 | 1.00 | 58.51 | C |
| ATOM | 2775 | C | LEU | A | 182 | −14.923 | 70.796 | 40.045 | 1.00 | 63.42 | C |
| ATOM | 2776 | O | LEU | A | 182 | −15.866 | 70.984 | 40.808 | 1.00 | 59.66 | O |
| ATOM | 2777 | N | SER | A | 183 | −13.907 | 71.636 | 39.939 | 1.00 | 68.63 | N |
| ATOM | 2779 | CA | SER | A | 183 | −13.815 | 72.766 | 40.849 | 1.00 | 74.67 | C |
| ATOM | 2781 | CB | SER | A | 183 | −12.618 | 72.624 | 41.763 | 1.00 | 74.42 | C |
| ATOM | 2784 | OG | SER | A | 183 | −13.083 | 72.150 | 43.012 | 1.00 | 88.15 | O |
| ATOM | 2786 | C | SER | A | 183 | −13.759 | 74.114 | 40.175 | 1.00 | 78.25 | C |
| ATOM | 2787 | O | SER | A | 183 | −13.306 | 74.245 | 39.039 | 1.00 | 88.01 | O |
| ATOM | 2788 | N | GLN | A | 184 | −14.248 | 75.112 | 40.893 | 1.00 | 78.87 | N |
| ATOM | 2790 | CA | GLN | A | 184 | −14.471 | 76.411 | 40.307 | 1.00 | 80.85 | C |
| ATOM | 2792 | CB | GLN | A | 184 | −15.040 | 77.350 | 41.353 | 1.00 | 84.43 | C |
| ATOM | 2795 | CG | GLN | A | 184 | −16.520 | 77.589 | 41.192 | 1.00 | 85.60 | C |
| ATOM | 2798 | CD | GLN | A | 184 | −16.830 | 79.061 | 41.209 | 1.00 | 84.94 | C |
| ATOM | 2799 | OE1 | GLN | A | 184 | −17.644 | 79.497 | 42.026 | 1.00 | 96.11 | O |
| ATOM | 2800 | NE2 | GLN | A | 184 | −16.143 | 79.839 | 40.355 | 1.00 | 61.70 | N |
| ATOM | 2803 | C | GLN | A | 184 | −13.145 | 76.937 | 39.821 | 1.00 | 82.64 | C |
| ATOM | 2804 | O | GLN | A | 184 | −12.197 | 76.965 | 40.582 | 1.00 | 87.02 | O |
| ATOM | 2805 | N | THR | A | 185 | −13.059 | 77.294 | 38.545 | 1.00 | 90.98 | N |
| ATOM | 2807 | CA | THR | A | 185 | −11.840 | 77.874 | 38.028 | 1.00 | 98.36 | C |
| ATOM | 2809 | CB | THR | A | 185 | −11.909 | 78.067 | 36.512 | 1.00 | 99.45 | C |
| ATOM | 2811 | OG1 | THR | A | 185 | −12.062 | 76.799 | 35.867 | 1.00 | 101.10 | O |
| ATOM | 2813 | CG2 | THR | A | 185 | −10.574 | 78.583 | 35.974 | 1.00 | 104.68 | C |
| ATOM | 2817 | C | THR | A | 185 | −11.712 | 79.212 | 38.715 | 1.00 | 107.41 | C |
| ATOM | 2818 | O | THR | A | 185 | −12.649 | 80.015 | 38.718 | 1.00 | 108.65 | O |
| ATOM | 2819 | N | SER | A | 186 | −10.557 | 79.415 | 39.338 | 1.00 | 117.87 | N |
| ATOM | 2821 | CA | SER | A | 186 | −10.289 | 80.643 | 40.071 | 1.00 | 124.73 | C |
| ATOM | 2823 | CB | SER | A | 186 | −10.109 | 80.316 | 41.563 | 1.00 | 124.08 | C |
| ATOM | 2826 | OG | SER | A | 186 | −8.733 | 80.080 | 41.868 | 1.00 | 132.77 | O |
| ATOM | 2828 | C | SER | A | 186 | −9.064 | 81.340 | 39.460 | 1.00 | 126.99 | C |
| ATOM | 2829 | O | SER | A | 186 | −8.465 | 82.221 | 40.072 | 1.00 | 128.13 | O |
| ATOM | 2830 | N | ASN | A | 187 | −8.705 | 80.934 | 38.244 | 1.00 | 131.93 | N |
| ATOM | 2832 | CA | ASN | A | 187 | −8.323 | 81.843 | 37.161 | 1.00 | 136.73 | C |
| ATOM | 2034 | CB | ASN | A | 187 | −8.183 | 81.046 | 35.870 | 1.00 | 134.75 | C |
| ATOM | 2837 | CG | ASN | A | 187 | −6.775 | 80.629 | 35.618 | 1.00 | 127.06 | C |
| ATOM | 2838 | OD1 | ASN | A | 187 | −5.875 | 80.998 | 36.377 | 1.00 | 103.34 | O |
| ATOM | 2839 | ND2 | ASN | A | 187 | −6.562 | 79.859 | 34.554 | 1.00 | 117.05 | N |
| ATOM | 2842 | C | ASN | A | 187 | −9.275 | 82.991 | 36.847 | 1.00 | 145.17 | C |
| ATOM | 2843 | O | ASN | A | 187 | −9.009 | 84.139 | 37.198 | 1.00 | 145.15 | O |
| ATOM | 2844 | N | VAL | A | 188 | −10.299 | 82.676 | 36.053 | 1.00 | 155.82 | N |
| ATOM | 2846 | CA | VAL | A | 188 | −11.605 | 83.343 | 36.087 | 1.00 | 162.11 | C |
| ATOM | 2848 | CB | VAL | A | 188 | −12.624 | 82.554 | 36.975 | 1.00 | 162.87 | C |
| ATOM | 2850 | CG1 | VAL | A | 188 | −12.800 | 83.179 | 38.380 | 1.00 | 159.86 | C |
| ATOM | 2854 | CG2 | VAL | A | 188 | −13.958 | 82.371 | 36.233 | 1.00 | 158.42 | C |
| ATOM | 2858 | C | VAL | A | 188 | −11.527 | 84.847 | 36.419 | 1.00 | 167.02 | C |
| ATOM | 2859 | O | VAL | A | 188 | −10.426 | 85.403 | 36.544 | 1.00 | 168.64 | O |
| ATOM | 2860 | N | ASP | A | 189 | −12.691 | 85.499 | 36.497 | 1.00 | 168.52 | N |
| ATOM | 2862 | CA | ASP | A | 189 | −12.860 | 86.930 | 36.198 | 1.00 | 166.34 | C |
| ATOM | 2864 | CB | ASP | A | 189 | −11.777 | 87.794 | 36.872 | 1.00 | 165.73 | C |
| ATOM | 2867 | CG | ASP | A | 189 | −12.088 | 88.103 | 38.334 | 1.00 | 156.94 | C |
| ATOM | 2868 | OD1 | ASP | A | 189 | −11.884 | 87.224 | 39.199 | 1.00 | 140.33 | O |
| ATOM | 2869 | OD2 | ASP | A | 189 | −12.524 | 89.207 | 38.718 | 1.00 | 145.89 | O |
| ATOM | 2870 | C | ASP | A | 189 | −12.989 | 87.249 | 34.693 | 1.00 | 165.91 | C |
| ATOM | 2871 | O | ASP | A | 189 | −13.996 | 87.833 | 34.283 | 1.00 | 164.90 | O |
| ATOM | 2872 | N | LYS | A | 190 | −11.999 | 86.865 | 33.879 | 1.00 | 164.47 | N |
| ATOM | 2874 | CA | LYS | A | 190 | −12.034 | 87.103 | 32.424 | 1.00 | 159.80 | C |
| ATOM | 2876 | CB | LYS | A | 190 | −11.098 | 88.276 | 32.015 | 1.00 | 157.48 | C |
| ATOM | 2879 | CG | LYS | A | 190 | −9.721 | 88.406 | 32.718 | 1.00 | 146.41 | C |
| ATOM | 2882 | CD | LYS | A | 190 | −8.908 | 89.611 | 32.194 | 1.00 | 126.91 | C |
| ATOM | 2885 | CE | LYS | A | 190 | −7.398 | 89.337 | 32.120 | 1.00 | 116.24 | C |
| ATOM | 2888 | NZ | LYS | A | 190 | −6.793 | 89.060 | 33.463 | 1.00 | 105.06 | N |
| ATOM | 2892 | C | LYS | A | 190 | −11.852 | 85.880 | 31.480 | 1.00 | 157.76 | C |
| ATOM | 2893 | O | LYS | A | 190 | −11.448 | 86.071 | 30.330 | 1.00 | 159.51 | O |
| ATOM | 2894 | N | GLU | A | 191 | −12.212 | 84.659 | 31.902 | 1.00 | 152.13 | N |
| ATOM | 2896 | CA | GLU | A | 191 | −11.650 | 83.413 | 31.334 | 1.00 | 144.60 | C |
| ATOM | 2898 | CB | GLU | A | 191 | −11.044 | 82.534 | 32.444 | 1.00 | 146.19 | C |
| ATOM | 2901 | CG | GLU | A | 191 | −9.571 | 82.793 | 32.744 | 1.00 | 143.52 | C |
| ATOM | 2904 | CD | GLU | A | 191 | −8.633 | 82.030 | 31.830 | 1.00 | 137.63 | C |
| ATOM | 2905 | OE1 | GLU | A | 191 | −8.605 | 80.784 | 31.933 | 1.00 | 128.49 | O |
| ATOM | 2906 | OE2 | GLU | A | 191 | −7.940 | 82.683 | 31.013 | 1.00 | 126.62 | O |
| ATOM | 2907 | C | GLU | A | 191 | −12.637 | 82.555 | 30.526 | 1.00 | 135.52 | C |
| ATOM | 2908 | O | GLU | A | 191 | −13.848 | 82.603 | 30.760 | 1.00 | 130.30 | O |
| ATOM | 2909 | N | GLU | A | 192 | −12.109 | 81.713 | 29.637 | 1.00 | 125.24 | N |
| ATOM | 2911 | CA | GLU | A | 192 | −12.944 | 81.030 | 28.646 | 1.00 | 123.29 | C |
| ATOM | 2913 | CB | GLU | A | 192 | −12.313 | 81.160 | 27.254 | 1.00 | 125.85 | C |
| ATOM | 2916 | CG | GLU | A | 192 | −13.283 | 81.572 | 26.149 | 1.00 | 126.49 | C |
| ATOM | 2919 | CD | GLU | A | 192 | −14.060 | 80.400 | 25.572 | 1.00 | 132.07 | C |
| ATOM | 2920 | OE1 | GLU | A | 192 | −14.856 | 79.782 | 26.320 | 1.00 | 123.75 | O |
| ATOM | 2921 | OE2 | GLU | A | 192 | −13.876 | 80.104 | 24.368 | 1.00 | 127.79 | O |
| ATOM | 2922 | C | GLU | A | 192 | −13.333 | 79.558 | 28.924 | 1.00 | 116.05 | C |
| ATOM | 2923 | O | GLU | A | 192 | −14.510 | 79.206 | 28.799 | 1.00 | 110.33 | O |
| ATOM | 2924 | N | GLU | A | 193 | −12.357 | 78.707 | 29.255 | 1.00 | 106.66 | N |
| ATOM | 2926 | CA | GLU | A | 193 | −12.591 | 77.288 | 29.565 | 1.00 | 95.70 | C |
| ATOM | 2928 | CB | GLU | A | 193 | −11.474 | 76.425 | 28.980 | 1.00 | 95.85 | C |
| ATOM | 2931 | CG | GLU | A | 193 | −10.103 | 76.754 | 29.560 | 1.00 | 108.23 | C |
| ATOM | 2934 | CD | GLU | A | 193 | −9.335 | 77.807 | 28.771 | 1.00 | 121.37 | C |
| ATOM | 2935 | OE1 | GLU | A | 193 | −9.614 | 79.019 | 28.939 | 1.00 | 123.05 | O |
| ATOM | 2936 | OE2 | GLU | A | 193 | −8.420 | 77.421 | 28.007 | 1.00 | 120.15 | O |
| ATOM | 2937 | C | GLU | A | 193 | −12.689 | 77.016 | 31.075 | 1.00 | 81.73 | C |
| ATOM | 2938 | O | GLU | A | 193 | −12.011 | 76.152 | 31.610 | 1.00 | 60.67 | O |
| ATOM | 2939 | N | ALA | A | 194 | −13.575 | 77.734 | 31.749 | 1.00 | 73.12 | N |
| ATOM | 2941 | CA | ALA | A | 194 | −13.558 | 77.822 | 33.198 | 1.00 | 72.63 | C |
| ATOM | 2943 | CB | ALA | A | 194 | −13.839 | 79.261 | 33.597 | 1.00 | 72.58 | C |
| ATOM | 2947 | C | ALA | A | 194 | −14.642 | 76.908 | 33.755 | 1.00 | 71.23 | C |
| ATOM | 2948 | O | ALA | A | 194 | −15.345 | 76.283 | 32.982 | 1.00 | 71.76 | O |
| ATOM | 2949 | N | VAL | A | 195 | −14.832 | 76.886 | 35.073 | 1.00 | 68.26 | N |
| ATOM | 2951 | CA | VAL | A | 195 | −16.049 | 76.364 | 35.680 | 1.00 | 60.57 | C |
| ATOM | 2953 | CB | VAL | A | 195 | −15.790 | 75.003 | 36.346 | 1.00 | 60.43 | C |
| ATOM | 2955 | CG1 | VAL | A | 195 | −16.991 | 74.557 | 37.220 | 1.00 | 57.97 | C |
| ATOM | 2959 | CG2 | VAL | A | 195 | −15.479 | 73.951 | 35.305 | 1.00 | 60.80 | C |
| ATOM | 2963 | C | VAL | A | 195 | −16.538 | 77.353 | 36.745 | 1.00 | 61.76 | C |
| ATOM | 2964 | O | VAL | A | 195 | −15.824 | 77.628 | 37.684 | 1.00 | 74.19 | O |
| ATOM | 2965 | N | THR | A | 196 | −17.729 | 77.917 | 36.600 | 1.00 | 58.68 | N |
| ATOM | 2967 | CA | THR | A | 196 | −18.225 | 78.909 | 37.531 | 1.00 | 58.31 | C |
| ATOM | 2969 | CB | THR | A | 196 | −18.294 | 80.333 | 36.878 | 1.00 | 53.01 | C |
| ATOM | 2971 | OG1 | THR | A | 196 | −19.250 | 80.341 | 35.831 | 1.00 | 61.65 | O |
| ATOM | 2973 | CG2 | THR | A | 196 | −17.029 | 80.716 | 36.118 | 1.00 | 53.52 | C |
| ATOM | 2977 | C | THR | A | 196 | −19.601 | 78.442 | 38.005 | 1.00 | 63.20 | C |
| ATOM | 2978 | O | THR | A | 196 | −20.344 | 77.801 | 37.271 | 1.00 | 61.20 | O |
| ATOM | 2979 | N | THR | A | 197 | −19.907 | 78.713 | 39.269 | 1.00 | 67.52 | N |
| ATOM | 2981 | CA | ILE | A | 197 | −21.093 | 78.164 | 39.912 | 1.00 | 63.16 | C |
| ATOM | 2983 | CB | ILE | A | 197 | −20.676 | 77.157 | 40.980 | 1.00 | 57.54 | C |
| ATOM | 2985 | CG1 | ILE | A | 197 | −19.630 | 76.209 | 40.380 | 1.00 | 62.47 | C |
| ATOM | 2988 | CD1 | ILE | A | 197 | −19.815 | 74.694 | 40.640 | 1.00 | 67.80 | C |
| ATOM | 2992 | CG2 | ILE | A | 197 | −21.930 | 76.503 | 41.622 | 1.00 | 41.75 | C |
| ATOM | 2996 | C | ILE | A | 197 | −21.861 | 79.304 | 40.568 | 1.00 | 67.57 | C |
| ATOM | 2997 | O | ILE | A | 197 | −21.247 | 80.146 | 41.203 | 1.00 | 68.15 | O |
| ATOM | 2998 | N | GLU | A | 198 | −23.178 | 79.349 | 40.362 | 1.00 | 73.72 | N |
| ATOM | 3000 | CA | GLU | A | 198 | −24.100 | 80.141 | 41.174 | 1.00 | 74.75 | C |
| ATOM | 3002 | CB | GLU | A | 198 | −25.059 | 80.984 | 40.317 | 1.00 | 72.96 | C |
| ATOM | 3005 | CG | GLU | A | 198 | −24.383 | 81.855 | 39.263 | 1.00 | 82.10 | C |
| ATOM | 3008 | CD | GLU | A | 198 | −23.862 | 83.182 | 39.799 | 1.00 | 95.31 | C |
| ATOM | 3009 | OE1 | GLU | A | 198 | −24.421 | 83.717 | 40.787 | 1.00 | 91.72 | O |
| ATOM | 3010 | OE2 | GLU | A | 198 | −22.896 | 83.708 | 39.201 | 1.00 | 102.90 | O |
| ATOM | 3011 | C | GLU | A | 198 | −24.910 | 79.173 | 42.023 | 1.00 | 81.23 | C |
| ATOM | 3012 | O | GLU | A | 198 | −26.018 | 78.758 | 41.664 | 1.00 | 80.07 | O |
| ATOM | 3013 | N | MET | A | 199 | −24.339 | 78.821 | 43.168 | 1.00 | 84.65 | N |
| ATOM | 3015 | CA | MET | A | 199 | −25.081 | 78.122 | 44.202 | 1.00 | 86.00 | C |
| ATOM | 3017 | CB | MET | A | 199 | −24.107 | 77.228 | 44.958 | 1.00 | 86.44 | C |
| ATOM | 3020 | CG | MET | A | 199 | −24.738 | 76.432 | 46.053 | 1.00 | 96.48 | C |
| ATOM | 3023 | SD | MET | A | 199 | −23.831 | 76.663 | 47.581 | 1.00 | 114.01 | S |
| ATOM | 3024 | CE | MET | A | 199 | −24.951 | 75.742 | 48.804 | 1.00 | 95.90 | C |
| ATOM | 3028 | C | MET | A | 199 | −25.743 | 79.151 | 45.129 | 1.00 | 89.88 | C |
| ATOM | 3029 | O | MET | A | 199 | −25.104 | 80.130 | 45.530 | 1.00 | 93.51 | O |
| ATOM | 3030 | N | ASN | A | 200 | −27.043 | 78.973 | 45.381 | 1.00 | 90.54 | N |
| ATOM | 3032 | CA | ASN | A | 200 | −27.773 | 79.613 | 46.485 | 1.00 | 84.65 | C |
| ATOM | 3034 | CB | ASN | A | 200 | −29.171 | 80.048 | 46.041 | 1.00 | 83.67 | C |
| ATOM | 3037 | CG | ASN | A | 200 | −29.152 | 81.081 | 44.944 | 1.00 | 81.50 | C |
| ATOM | 3038 | OD1 | ASN | A | 200 | −28.119 | 81.691 | 44.642 | 1.00 | 84.51 | O |
| ATOM | 3039 | ND2 | ASN | A | 200 | −30.315 | 81.288 | 44.340 | 1.00 | 81.37 | N |
| ATOM | 3042 | C | ASN | A | 200 | −28.016 | 78.625 | 47.602 | 1.00 | 81.42 | C |
| ATOM | 3043 | O | ASN | A | 200 | −28.349 | 79.007 | 48.712 | 1.00 | 81.18 | O |
| ATOM | 3044 | N | GLU | A | 201 | −27.993 | 77.351 | 47.237 | 1.00 | 84.50 | N |
| ATOM | 3046 | CA | GLU | A | 201 | −28.532 | 76.270 | 48.052 | 1.00 | 84.70 | C |
| ATOM | 3048 | CB | GLU | A | 201 | −30.062 | 76.113 | 47.902 | 1.00 | 77.57 | C |
| ATOM | 3051 | CG | GLU | A | 201 | −30.915 | 77.305 | 48.314 | 1.00 | 77.09 | C |
| ATOM | 3054 | CD | GLU | A | 201 | −31.979 | 77.691 | 47.277 | 1.00 | 93.59 | C |
| ATOM | 3055 | OE1 | GLU | A | 201 | −33.066 | 77.072 | 47.247 | 1.00 | 85.46 | O |
| ATOM | 3056 | OE2 | GLU | A | 201 | −31.770 | 78.635 | 46.477 | 1.00 | 89.94 | O |
| ATOM | 3057 | C | GLU | A | 201 | −27.798 | 75.036 | 47.521 | 1.00 | 84.81 | C |
| ATOM | 3058 | O | GLU | A | 201 | −27.666 | 74.862 | 46.317 | 1.00 | 71.90 | O |
| ATOM | 3059 | N | PRO | A | 202 | −27.327 | 74.168 | 48.407 | 1.00 | 92.08 | N |
| ATOM | 3060 | CA | PRO | A | 202 | −26.802 | 72.879 | 47.960 | 1.00 | 92.72 | C |
| ATOM | 3062 | CB | PRO | A | 202 | −26.444 | 72.178 | 49.274 | 1.00 | 95.40 | C |
| ATOM | 3065 | CG | PRO | A | 202 | −26.425 | 73.242 | 50.312 | 1.00 | 95.01 | C |
| ATOM | 3068 | CD | PRO | A | 202 | −27.406 | 74.260 | 49.875 | 1.00 | 94.01 | C |
| ATOM | 3071 | C | PRO | A | 202 | −27.937 | 72.137 | 47.252 | 1.00 | 92.36 | C |
| ATOM | 3072 | O | PRO | A | 202 | −29.018 | 72.002 | 47.836 | 1.00 | 92.36 | O |
| ATOM | 3073 | N | VAL | A | 203 | −27.722 | 71.682 | 46.020 | 1.00 | 87.48 | N |
| ATOM | 3075 | CA | VAL | A | 203 | −28.605 | 70.674 | 45.451 | 1.00 | 81.95 | C |
| ATOM | 3077 | CB | VAL | A | 203 | −29.068 | 71.040 | 44.049 | 1.00 | 80.98 | C |
| ATOM | 3079 | CG1 | VAL | A | 203 | −28.118 | 70.439 | 43.027 | 1.00 | 87.42 | C |
| ATOM | 3083 | CG2 | VAL | A | 203 | −30.466 | 70.485 | 43.825 | 1.00 | 85.16 | C |
| ATOM | 3087 | C | VAL | A | 203 | −27.930 | 69.317 | 45.354 | 1.00 | 77.81 | C |
| ATOM | 3088 | O | VAL | A | 203 | −26.716 | 69.231 | 45.221 | 1.00 | 84.54 | O |
| ATOM | 3089 | N | GLN | A | 204 | −28.720 | 68.254 | 45.380 | 1.00 | 72.55 | N |
| ATOM | 3091 | CA | GLN | A | 204 | −28.220 | 66.966 | 44.950 | 1.00 | 67.19 | C |
| ATOM | 3093 | CB | GLN | A | 204 | −27.601 | 66.236 | 46.146 | 1.00 | 66.66 | C |
| ATOM | 3096 | CG | GLN | A | 204 | −27.928 | 64.729 | 46.223 | 1.00 | 79.79 | C |
| ATOM | 3099 | CD | GLN | A | 204 | −26.813 | 63.883 | 46.846 | 1.00 | 75.05 | C |
| ATOM | 3100 | OE1 | GLN | A | 204 | −25.669 | 64.330 | 46.946 | 1.00 | 75.66 | O |
| ATOM | 3101 | NE2 | GLN | A | 204 | −27.142 | 62.655 | 47.239 | 1.00 | 66.02 | N |
| ATOM | 3104 | C | GLN | A | 204 | −29.298 | 66.143 | 44.242 | 1.00 | 67.05 | C |
| ATOM | 3105 | O | GLN | A | 204 | −30.278 | 65.707 | 44.841 | 1.00 | 67.35 | O |
| ATOM | 3106 | N | LEU | A | 205 | −29.133 | 65.923 | 42.948 | 1.00 | 63.18 | N |
| ATOM | 3108 | CA | LEU | A | 205 | −30.197 | 65.250 | 42.214 | 1.00 | 69.17 | C |
| ATOM | 3110 | CB | LEU | A | 205 | −30.939 | 66.273 | 41.348 | 1.00 | 75.41 | C |
| ATOM | 3113 | CG | LEU | A | 205 | −32.117 | 67.102 | 41.867 | 1.00 | 88.37 | C |
| ATOM | 3115 | CD1 | LEU | A | 205 | −32.248 | 68.318 | 40.969 | 1.00 | 90.68 | C |
| ATOM | 3119 | CD2 | LEU | A | 205 | −33.456 | 66.321 | 41.889 | 1.00 | 97.32 | C |
| ATOM | 3123 | C | LEU | A | 205 | −29.672 | 64.130 | 41.319 | 1.00 | 60.08 | C |
| ATOM | 3124 | O | LEU | A | 205 | −28.483 | 63.903 | 41.213 | 1.00 | 58.11 | O |
| ATOM | 3125 | N | THR | A | 206 | −30.568 | 63.450 | 40.633 | 1.00 | 59.08 | N |
| ATOM | 3127 | CA | THR | A | 206 | −30.242 | 62.153 | 40.062 | 1.00 | 65.08 | C |
| ATOM | 3129 | CB | THR | A | 206 | −30.792 | 61.013 | 40.967 | 1.00 | 66.40 | C |
| ATOM | 3131 | OG1 | THR | A | 206 | −29.935 | 60.815 | 42.092 | 1.00 | 65.23 | O |
| ATOM | 3133 | CG2 | THR | A | 206 | −30.777 | 59.652 | 40.259 | 1.00 | 64.09 | C |
| ATOM | 3137 | C | THR | A | 206 | −30.920 | 62.074 | 38.695 | 1.00 | 67.80 | C |
| ATOM | 3138 | O | THR | A | 206 | −32.148 | 62.197 | 38.592 | 1.00 | 69.63 | O |
| ATOM | 3139 | N | PHE | A | 207 | −30.135 | 61.824 | 37.654 | 1.00 | 64.98 | N |
| ATOM | 3141 | CA | PHE | A | 207 | −30.699 | 61.751 | 36.322 | 1.00 | 58.78 | C |
| ATOM | 3143 | CB | PHE | A | 207 | −30.266 | 62.985 | 35.579 | 1.00 | 59.24 | C |
| ATOM | 3146 | CG | PHE | A | 207 | −30.672 | 64.247 | 36.271 | 1.00 | 56.51 | C |
| ATOM | 3147 | CD1 | PHE | A | 207 | −31.931 | 64.776 | 36.067 | 1.00 | 47.08 | C |
| ATOM | 3149 | CE1 | PHE | A | 207 | −32.296 | 65.943 | 36.673 | 1.00 | 52.64 | C |
| ATOM | 3151 | CZ | PHE | A | 207 | −31.418 | 66.595 | 37.520 | 1.00 | 49.29 | C |
| ATOM | 3153 | CE2 | PHE | A | 207 | −30.170 | 66.063 | 37.750 | 1.00 | 65.74 | C |
| ATOM | 3155 | CD2 | PHE | A | 207 | −29.812 | 64.877 | 37.148 | 1.00 | 53.13 | C |
| ATOM | 3157 | C | PHE | A | 207 | −30.166 | 60.547 | 35.617 | 1.00 | 57.45 | C |
| ATOM | 3158 | O | PHE | A | 207 | −29.045 | 60.133 | 35.914 | 1.00 | 65.69 | O |
| ATOM | 3159 | N | ALA | A | 208 | −30.966 | 60.001 | 34.705 | 1.00 | 53.47 | N |
| ATOM | 3161 | CA | ALA | A | 208 | −30.511 | 58.998 | 33.729 | 1.00 | 53.26 | C |
| ATOM | 3163 | CB | ALA | A | 208 | −31.714 | 58.352 | 33.008 | 1.00 | 55.60 | C |
| ATOM | 3167 | C | ALA | A | 208 | −29.505 | 59.530 | 32.693 | 1.00 | 51.47 | C |
| ATOM | 3168 | O | ALA | A | 208 | −29.745 | 60.491 | 31.962 | 1.00 | 45.24 | O |
| ATOM | 3169 | N | LEU | A | 209 | −28.368 | 58.856 | 32.614 | 1.00 | 50.90 | N |
| ATOM | 3171 | CA | LEU | A | 209 | −27.322 | 59.224 | 31.673 | 1.00 | 42.80 | C |
| ATOM | 3173 | CB | LEU | A | 209 | −26.083 | 58.390 | 31.989 | 1.00 | 36.88 | C |
| ATOM | 3176 | CG | LEU | A | 209 | −25.348 | 58.803 | 33.252 | 1.00 | 40.18 | C |
| ATOM | 3178 | CD1 | LEU | A | 209 | −24.381 | 57.721 | 33.668 | 1.00 | 58.13 | C |
| ATOM | 3182 | CD2 | LEU | A | 209 | −24.593 | 60.120 | 33.060 | 1.00 | 52.96 | C |
| ATOM | 3186 | C | LEU | A | 209 | −27.733 | 59.019 | 30.205 | 1.00 | 44.82 | C |
| ATOM | 3187 | O | LEU | A | 209 | −27.131 | 59.610 | 29.305 | 1.00 | 43.03 | O |
| ATOM | 3188 | N | ARG | A | 210 | −28.739 | 58.188 | 29.941 | 1.00 | 43.66 | N |
| ATOM | 3190 | CA | ARG | A | 210 | −29.132 | 57.959 | 28.552 | 1.00 | 47.76 | C |
| ATOM | 3192 | CB | AEG | A | 210 | −30.320 | 56.989 | 28.446 | 1.00 | 55.99 | C |
| ATOM | 3195 | CG | AEG | A | 210 | −30.283 | 55.980 | 27.289 | 1.00 | 56.35 | C |
| ATOM | 3198 | CD | ARG | A | 210 | −31.660 | 55.647 | 26.657 | 1.00 | 73.16 | C |
| ATOM | 3201 | NE | ARG | A | 210 | −31.533 | 55.259 | 25.242 | 1.00 | 86.03 | N |
| ATOM | 3203 | CZ | ARG | A | 210 | −30.561 | 54.466 | 24.755 | 1.00 | 94.59 | C |
| ATOM | 3204 | NH1 | ARG | A | 210 | −29.628 | 53.953 | 25.562 | 1.00 | 98.09 | N |
| ATOM | 3207 | NH2 | ARG | A | 210 | −30.519 | 54.156 | 23.459 | 1.00 | 63.39 | N |
| ATOM | 3210 | C | ARG | A | 210 | −29.523 | 59.312 | 27.959 | 1.00 | 51.99 | C |
| ATOM | 3211 | O | ARG | A | 210 | −29.096 | 59.666 | 26.866 | 1.00 | 49.05 | O |
| ATOM | 3212 | N | TYR | A | 211 | −30.355 | 60.066 | 28.673 | 1.00 | 52.01 | N |
| ATOM | 3214 | CA | TYR | A | 211 | −30.884 | 61.299 | 28.100 | 1.00 | 49.90 | C |
| ATOM | 3216 | CB | TYR | A | 211 | −32.125 | 61.782 | 28.856 | 1.00 | 45.42 | C |
| ATOM | 3219 | CG | TYR | A | 211 | −33.311 | 60.844 | 28.728 | 1.00 | 50.38 | C |
| ATOM | 3220 | CD1 | TYR | A | 211 | −34.257 | 60.992 | 27.721 | 1.00 | 48.63 | C |
| ATOM | 3222 | CE1 | TYR | A | 211 | −35.342 | 60.115 | 27.625 | 1.00 | 51.31 | C |
| ATOM | 3224 | CZ | TYR | A | 211 | −35.485 | 59.077 | 28.535 | 1.00 | 69.42 | C |
| ATOM | 3225 | OH | TYR | A | 211 | −36.564 | 58.188 | 28.513 | 1.00 | 58.42 | O |
| ATOM | 3227 | CE2 | TYR | A | 211 | −34.527 | 58.915 | 29.507 | 1.00 | 64.53 | C |
| ATOM | 3229 | CD2 | TYR | A | 211 | −33.462 | 59.782 | 29.598 | 1.00 | 57.08 | C |
| ATOM | 3231 | C | TYR | A | 211 | −29.776 | 62.346 | 28.108 | 1.00 | 47.19 | C |
| ATOM | 3232 | O | TYR | A | 211 | −29.672 | 63.159 | 27.192 | 1.00 | 47.17 | O |
| ATOM | 3233 | N | LEU | A | 212 | −28.903 | 62.291 | 29.105 | 1.00 | 36.43 | N |
| ATOM | 3235 | CA | LEU | A | 212 | −27.836 | 63.269 | 29.167 | 1.00 | 37.77 | C |
| ATOM | 3237 | CB | LEU | A | 212 | −27.077 | 63.132 | 30.488 | 1.00 | 38.80 | C |
| ATOM | 3240 | CG | LEU | A | 212 | −27.781 | 63.882 | 31.620 | 1.00 | 37.62 | C |
| ATOM | 3242 | CD1 | LEU | A | 212 | −27.026 | 63.738 | 32.913 | 1.00 | 49.06 | C |
| ATOM | 3246 | CD2 | LEU | A | 212 | −27.865 | 65.343 | 31.263 | 1.00 | 48.06 | C |
| ATOM | 3250 | C | LEU | A | 212 | −26.922 | 63.116 | 27.949 | 1.00 | 40.61 | C |
| ATOM | 3251 | O | LEU | A | 212 | −26.444 | 64.093 | 27.383 | 1.00 | 36.12 | O |
| ATOM | 3252 | N | ASN | A | 213 | −26.726 | 61.890 | 27.482 | 1.00 | 37.27 | N |
| ATOM | 3254 | CA | ASN | A | 213 | −25.835 | 61.698 | 26.356 | 1.00 | 28.93 | C |
| ATOM | 3256 | CB | ASN | A | 213 | −25.376 | 60.253 | 26.340 | 1.00 | 39.03 | C |
| ATOM | 3259 | CG | ASN | A | 213 | −24.315 | 59.976 | 27.362 | 1.00 | 35.47 | C |
| ATOM | 3260 | OD1 | ASN | A | 213 | −23.279 | 60.615 | 27.344 | 1.00 | 36.21 | O |
| ATOM | 3261 | ND2 | ASN | A | 213 | −24.544 | 58.989 | 28.220 | 1.00 | 53.66 | N |
| ATOM | 3264 | C | ASN | A | 213 | −26.511 | 61.946 | 25.036 | 1.00 | 34.01 | C |
| ATOM | 3265 | O | ASN | A | 213 | −25.864 | 61.974 | 23.981 | 1.00 | 47.25 | O |
| ATOM | 3266 | N | PHE | A | 214 | −27.833 | 62.016 | 25.050 | 1.00 | 35.49 | N |
| ATOM | 3268 | CA | PHE | A | 214 | −28.516 | 62.590 | 23.907 | 1.00 | 35.51 | C |
| ATOM | 3270 | CB | PHE | A | 214 | −30.010 | 62.272 | 23.964 | 1.00 | 37.11 | C |
| ATOM | 3273 | CG | PHE | A | 214 | −30.409 | 60.891 | 23.498 | 1.00 | 42.68 | C |
| ATOM | 3274 | CD1 | PHE | A | 214 | −30.218 | 60.492 | 22.176 | 1.00 | 62.35 | C |
| ATOM | 3276 | CE1 | PHE | A | 214 | −30.655 | 59.229 | 21.725 | 1.00 | 56.81 | C |
| ATOM | 3278 | CZ | PHE | A | 214 | −31.305 | 58.366 | 22.614 | 1.00 | 47.74 | C |
| ATOM | 3280 | CE2 | PHE | A | 214 | −31.558 | 58.788 | 23.947 | 1.00 | 36.47 | C |
| ATOM | 3282 | CD2 | PHE | A | 214 | −31.133 | 60.049 | 24.363 | 1.00 | 40.35 | C |
| ATOM | 3284 | C | PHE | A | 214 | −28.275 | 64.128 | 23.929 | 1.00 | 35.80 | C |
| ATOM | 3285 | O | PHE | A | 214 | −28.004 | 64.713 | 22.905 | 1.00 | 33.00 | O |
| ATOM | 3286 | N | PHE | A | 215 | −28.339 | 64.805 | 25.070 | 1.00 | 34.10 | N |
| ATOM | 3288 | CA | PHE | A | 215 | −28.221 | 66.258 | 25.043 | 1.00 | 39.49 | C |
| ATOM | 3290 | CB | PHE | A | 215 | −28.399 | 66.859 | 26.454 | 1.00 | 47.83 | C |
| ATOM | 3293 | CG | PHE | A | 215 | −29.683 | 66.504 | 27.138 | 1.00 | 37.82 | C |
| ATOM | 3294 | CD1 | PHE | A | 215 | −30.740 | 65.999 | 26.424 | 1.00 | 34.89 | C |
| ATOM | 3296 | CE1 | PHE | A | 215 | −31.905 | 65.665 | 27.052 | 1.00 | 33.32 | C |
| ATOM | 3298 | CZ | PHE | A | 215 | −32.048 | 65.861 | 28.405 | 1.00 | 32.59 | C |
| ATOM | 3300 | CE2 | PHE | A | 215 | −31.026 | 66.461 | 29.121 | 1.00 | 40.70 | C |
| ATOM | 3302 | CD2 | PHE | A | 215 | −29.855 | 66.781 | 28.482 | 1.00 | 38.10 | C |
| ATOM | 3304 | C | PHE | A | 215 | −26.835 | 66.714 | 24.517 | 1.00 | 44.30 | C |
| ATOM | 3305 | O | PHE | A | 215 | −26.662 | 67.837 | 23.994 | 1.00 | 43.39 | O |
| ATOM | 3306 | N | THR | A | 216 | −25.816 | 65.892 | 24.742 | 1.00 | 36.07 | N |
| ATOM | 3308 | CA | THR | A | 216 | −24.479 | 66.300 | 24.389 | 1.00 | 33.31 | C |
| ATOM | 3310 | CB | THR | A | 216 | −23.483 | 65.524 | 25.209 | 1.00 | 30.36 | C |
| ATOM | 3312 | OG1 | THR | A | 216 | −23.669 | 64.113 | 24.996 | 1.00 | 38.23 | O |
| ATOM | 3314 | CG2 | THR | A | 216 | −23.746 | 65.804 | 26.676 | 1.00 | 17.26 | C |
| ATOM | 3318 | C | THR | A | 216 | −24.251 | 66.083 | 22.887 | 1.00 | 39.67 | C |
| ATOM | 3319 | O | THR | A | 216 | −23.142 | 66.308 | 22.370 | 1.00 | 35.59 | O |
| ATOM | 3320 | N | LYS | A | 217 | −25.320 | 65.753 | 22.161 | 1.00 | 30.97 | N |
| ATOM | 3322 | CA | LYS | A | 217 | −25.266 | 65.955 | 20.712 | 1.00 | 35.17 | C |
| ATOM | 3324 | CB | LYS | A | 217 | −26.286 | 65.143 | 19.907 | 1.00 | 24.15 | C |
| ATOM | 3327 | CG | LYS | A | 217 | −26.294 | 63.661 | 20.326 | 1.00 | 45.84 | C |
| ATOM | 3330 | CD | LYS | A | 217 | −25.773 | 62.619 | 19.341 | 1.00 | 55.58 | C |
| ATOM | 3333 | CE | LYS | A | 217 | −24.324 | 62.220 | 19.644 | 1.00 | 70.28 | C |
| ATOM | 3336 | NZ | LYS | A | 217 | −24.131 | 60.815 | 20.132 | 1.00 | 72.21 | N |
| ATOM | 3340 | C | LYS | A | 217 | −25.190 | 67.402 | 20.301 | 1.00 | 32.47 | C |
| ATOM | 3341 | O | LYS | A | 217 | −24.784 | 67.677 | 19.162 | 1.00 | 35.68 | O |
| ATOM | 3342 | N | ALA | A | 218 | −25.384 | 68.279 | 21.283 | 1.00 | 34.47 | N |
| ATOM | 3344 | CA | ALA | A | 218 | −25.446 | 69.718 | 21.074 | 1.00 | 39.29 | C |
| ATOM | 3346 | CB | ALA | A | 218 | −26.315 | 70.334 | 22.093 | 1.00 | 46.57 | C |
| ATOM | 3350 | C | ALA | A | 218 | −24.087 | 70.359 | 21.191 | 1.00 | 40.26 | C |
| ATOM | 3351 | O | ALA | A | 218 | −23.938 | 71.574 | 21.049 | 1.00 | 48.36 | O |
| ATOM | 3352 | N | THR | A | 219 | −23.094 | 69.541 | 21.482 | 1.00 | 39.02 | N |
| ATOM | 3354 | CA | THR | A | 219 | −21.790 | 70.079 | 21.822 | 1.00 | 40.34 | C |
| ATOM | 3356 | CB | THR | A | 219 | −20.893 | 68.916 | 22.214 | 1.00 | 38.96 | C |
| ATOM | 3358 | OG1 | THR | A | 219 | −21.558 | 68.187 | 23.252 | 1.00 | 41.19 | O |
| ATOM | 3360 | CG2 | THR | A | 219 | −19.680 | 69.404 | 22.917 | 1.00 | 31.79 | C |
| ATOM | 3364 | C | THR | A | 219 | −21.194 | 70.968 | 20.722 | 1.00 | 35.58 | C |
| ATOM | 3365 | O | THR | A | 219 | −20.701 | 72.063 | 20.960 | 1.00 | 46.18 | O |
| ATOM | 3366 | N | PRO | A | 220 | −21.278 | 70.557 | 19.477 | 1.00 | 35.58 | N |
| ATOM | 3367 | CA | PRO | A | 220 | −20.680 | 71.349 | 18.410 | 1.00 | 30.85 | C |
| ATOM | 3369 | CB | PRO | A | 220 | −21.093 | 70.564 | 17.184 | 1.00 | 23.78 | C |
| ATOM | 3372 | CG | PRO | A | 220 | −21.081 | 69.215 | 17.671 | 1.00 | 23.55 | C |
| ATOM | 3375 | CD | PRO | A | 220 | −21.882 | 69.332 | 18.939 | 1.00 | 38.57 | C |
| ATOM | 3378 | C | PRO | A | 220 | −21.206 | 72.778 | 18.381 | 1.00 | 35.58 | C |
| ATOM | 3379 | O | PRO | A | 220 | −20.568 | 73.654 | 17.823 | 1.00 | 38.64 | O |
| ATOM | 3380 | N | LEU | A | 221 | −22.340 | 73.028 | 19.017 | 1.00 | 42.51 | N |
| ATOM | 3382 | CA | LEU | A | 221 | −22.929 | 74.352 | 18.949 | 1.00 | 42.01 | C |
| ATOM | 3384 | CB | LEU | A | 221 | −24.416 | 74.270 | 19.287 | 1.00 | 37.67 | C |
| ATOM | 3387 | CG | LEU | A | 221 | −25.301 | 73.703 | 18.174 | 1.00 | 42.22 | C |
| ATOM | 3389 | CD1 | LEU | A | 221 | −26.646 | 73.411 | 18.757 | 1.00 | 36.67 | C |
| ATOM | 3393 | CD2 | LEU | A | 221 | −25.478 | 74.645 | 16.988 | 1.00 | 40.60 | C |
| ATOM | 3397 | C | LEU | A | 221 | −22.218 | 75.381 | 19.847 | 1.00 | 40.30 | C |
| ATOM | 3398 | O | LEU | A | 221 | −22.295 | 76.575 | 19.630 | 1.00 | 43.90 | O |
| ATOM | 3399 | N | SER | A | 222 | −21.604 | 74.973 | 20.936 | 1.00 | 40.67 | N |
| ATOM | 3401 | CA | SER | A | 222 | −21.005 | 75.991 | 21.765 | 1.00 | 40.69 | C |
| ATOM | 3403 | CB | SER | A | 222 | −22.018 | 76.580 | 22.709 | 1.00 | 44.19 | C |
| ATOM | 3406 | OG | SER | A | 222 | −21.382 | 77.098 | 23.855 | 1.00 | 63.05 | O |
| ATOM | 3408 | C | SER | A | 222 | −19.907 | 75.376 | 22.565 | 1.00 | 47.59 | C |
| ATOM | 3409 | O | SER | A | 222 | −19.983 | 74.211 | 22.972 | 1.00 | 44.05 | O |
| ATOM | 3410 | N | SER | A | 223 | −18.852 | 76.158 | 22.737 | 1.00 | 50.17 | N |
| ATOM | 3412 | CA | SER | A | 223 | −17.703 | 75.698 | 23.518 | 1.00 | 53.78 | C |
| ATOM | 3414 | CB | SER | A | 223 | −16.563 | 76.726 | 23.399 | 1.00 | 57.96 | C |
| ATOM | 3417 | OG | SER | A | 223 | −16.723 | 77.791 | 24.337 | 1.00 | 78.27 | O |
| ATOM | 3419 | C | SER | A | 223 | −18.087 | 75.412 | 24.990 | 1.00 | 47.28 | C |
| ATOM | 3420 | O | SER | A | 223 | −17.382 | 74.695 | 25.726 | 1.00 | 36.08 | O |
| ATOM | 3421 | N | THR | A | 224 | −19.257 | 75.906 | 25.385 | 1.00 | 44.37 | N |
| ATOM | 3423 | CA | THR | A | 224 | −19.596 | 76.005 | 26.799 | 1.00 | 51.74 | C |
| ATOM | 3425 | CB | THR | A | 224 | −19.218 | 77.416 | 27.345 | 1.00 | 47.25 | C |
| ATOM | 3427 | OG1 | THR | A | 224 | −20.032 | 77.719 | 28.471 | 1.00 | 48.93 | O |
| ATOM | 3429 | CG2 | THR | A | 224 | −19.571 | 78.506 | 26.363 | 1.00 | 59.16 | C |
| ATOM | 3433 | C | THR | A | 224 | −21.070 | 75.646 | 27.032 | 1.00 | 48.73 | C |
| ATOM | 3434 | O | THR | A | 224 | −21.885 | 75.738 | 26.115 | 1.00 | 48.88 | O |
| ATOM | 3435 | N | VAL | A | 225 | −21.383 | 75.163 | 28.235 | 1.00 | 47.00 | N |
| ATOM | 3437 | CA | VAL | A | 225 | −22.688 | 74.576 | 28.542 | 1.00 | 42.05 | C |
| ATOM | 3439 | CB | VAL | A | 225 | −22.643 | 73.056 | 28.564 | 1.00 | 43.28 | C |
| ATOM | 3441 | CG1 | VAL | A | 225 | −21.854 | 72.566 | 29.796 | 1.00 | 42.86 | C |
| ATOM | 3445 | CG2 | VAL | A | 225 | −24.058 | 72.469 | 28.519 | 1.00 | 47.58 | C |
| ATOM | 3449 | C | VAL | A | 225 | −23.056 | 74.979 | 29.955 | 1.00 | 46.82 | C |
| ATOM | 3450 | O | VAL | A | 225 | −22.158 | 75.255 | 30.743 | 1.00 | 50.95 | O |
| ATOM | 3451 | N | THR | A | 226 | −24.348 | 75.027 | 30.278 | 1.00 | 49.56 | N |
| ATOM | 3453 | CA | THR | A | 226 | −24.765 | 75.412 | 31.621 | 1.00 | 57.64 | C |
| ATOM | 3455 | CB | THR | A | 226 | −25.236 | 76.912 | 31.732 | 1.00 | 61.75 | C |
| ATOM | 3457 | OG1 | THR | A | 226 | −26.625 | 77.029 | 31.389 | 1.00 | 78.71 | O |
| ATOM | 3459 | CG2 | THR | A | 226 | −24.517 | 77.811 | 30.705 | 1.00 | 54.95 | C |
| ATOM | 3463 | C | THR | A | 226 | −25.826 | 74.451 | 32.089 | 1.00 | 54.06 | C |
| ATOM | 3464 | O | THR | A | 226 | −26.611 | 73.922 | 31.308 | 1.00 | 57.41 | O |
| ATOM | 3465 | N | LEU | A | 227 | −25.792 | 74.174 | 33.379 | 1.00 | 56.59 | N |
| ATOM | 3467 | CA | LEU | A | 227 | −26.716 | 73.212 | 33.950 | 1.00 | 55.05 | C |
| ATOM | 3469 | CB | LEU | A | 227 | −25.997 | 71.978 | 34.540 | 1.00 | 56.54 | C |
| ATOM | 3472 | CG | LEU | A | 227 | −24.696 | 71.488 | 33.900 | 1.00 | 53.60 | C |
| ATOM | 3474 | CD1 | LEU | A | 227 | −23.857 | 70.739 | 34.902 | 1.00 | 55.99 | C |
| ATOM | 3478 | CD2 | LEU | A | 227 | −24.990 | 70.612 | 32.709 | 1.00 | 54.43 | C |
| ATOM | 3482 | C | LEU | A | 227 | −27.382 | 73.995 | 35.050 | 1.00 | 54.58 | C |
| ATOM | 3483 | O | LEU | A | 227 | −26.733 | 74.597 | 35.902 | 1.00 | 59.07 | O |
| ATOM | 3484 | N | SER | A | 228 | −28.696 | 74.019 | 35.022 | 1.00 | 58.69 | N |
| ATOM | 3486 | CA | SER | A | 228 | −29.389 | 74.744 | 36.051 | 1.00 | 55.94 | C |
| ATOM | 3488 | CB | SER | A | 228 | −30.283 | 75.798 | 35.429 | 1.00 | 62.92 | C |
| ATOM | 3491 | OG | SER | A | 228 | −29.460 | 76.805 | 34.864 | 1.00 | 59.98 | O |
| ATOM | 3493 | C | SER | A | 228 | −30.172 | 73.688 | 36.740 | 1.00 | 54.03 | C |
| ATOM | 3494 | O | SER | A | 228 | −30.680 | 72.780 | 36.102 | 1.00 | 52.95 | O |
| ATOM | 3495 | N | MET | A | 229 | −30.167 | 73.782 | 38.058 | 1.00 | 61.64 | N |
| ATOM | 3497 | CA | MET | A | 229 | −30.637 | 72.721 | 38.915 | 1.00 | 66.74 | C |
| ATOM | 3499 | CB | MET | A | 229 | −29.442 | 71.948 | 39.442 | 1.00 | 68.75 | C |
| ATOM | 3502 | CG | MET | A | 229 | −29.395 | 70.527 | 38.926 | 1.00 | 75.71 | C |
| ATOM | 3505 | SD | MET | A | 229 | −27.811 | 69.745 | 39.235 | 1.00 | 66.48 | S |
| ATOM | 3506 | CE | MET | A | 229 | −26.696 | 71.141 | 39.101 | 1.00 | 52.55 | C |
| ATOM | 3510 | C | MET | A | 229 | −31.346 | 73.359 | 40.083 | 1.00 | 66.53 | C |
| ATOM | 3511 | O | MET | A | 229 | −30.851 | 74.349 | 40.614 | 1.00 | 69.85 | O |
| ATOM | 3512 | N | SER | A | 230 | −32.471 | 72.772 | 40.490 | 1.00 | 65.11 | N |
| ATOM | 3514 | CA | SER | A | 230 | −33.096 | 73.087 | 41.771 | 1.00 | 63.19 | C |
| ATOM | 3516 | CB | SER | A | 230 | −34.034 | 74.298 | 41.632 | 1.00 | 62.76 | C |
| ATOM | 3519 | OG | SER | A | 230 | −33.422 | 75.486 | 42.126 | 1.00 | 58.20 | O |
| ATOM | 3521 | C | SER | A | 230 | −33.873 | 71.893 | 42.322 | 1.00 | 65.01 | C |
| ATOM | 3522 | O | SER | A | 230 | −34.026 | 70.859 | 41.687 | 1.00 | 55.74 | O |
| ATOM | 3523 | N | ALA | A | 231 | −34.365 | 72.053 | 43.537 | 1.00 | 72.76 | N |
| ATOM | 3525 | CA | ALA | A | 231 | −34.694 | 70.919 | 44.358 | 1.00 | 77.15 | C |
| ATOM | 3527 | CB | ALA | A | 231 | −35.232 | 71.401 | 45.709 | 1.00 | 85.37 | C |
| ATOM | 3531 | C | ALA | A | 231 | −35.666 | 69.978 | 43.637 | 1.00 | 78.96 | C |
| ATOM | 3532 | O | ALA | A | 231 | −35.356 | 68.798 | 43.515 | 1.00 | 87.19 | O |
| ATOM | 3533 | N | ASP | A | 232 | −36.824 | 70.424 | 43.157 | 1.00 | 74.50 | N |
| ATOM | 3535 | CA | ASP | A | 232 | −37.824 | 69.427 | 42.745 | 1.00 | 81.28 | C |
| ATOM | 3537 | CB | ASP | A | 232 | −38.977 | 69.357 | 43.763 | 1.00 | 88.07 | C |
| ATOM | 3540 | CG | ASP | A | 232 | −39.390 | 67.921 | 44.108 | 1.00 | 92.86 | C |
| ATOM | 3541 | OD1 | ASP | A | 232 | −38.816 | 67.358 | 45.067 | 1.00 | 92.93 | O |
| ATOM | 3542 | OD2 | ASP | A | 232 | −40.286 | 67.287 | 43.503 | 1.00 | 85.58 | O |
| ATOM | 3543 | C | ASP | A | 232 | −38.360 | 69.646 | 41.321 | 1.00 | 80.86 | C |
| ATOM | 3544 | O | ASP | A | 232 | −39.564 | 69.560 | 41.042 | 1.00 | 74.07 | O |
| ATOM | 3545 | N | VAL | A | 233 | −37.430 | 69.927 | 40.419 | 1.00 | 77.38 | N |
| ATOM | 3547 | CA | VAL | A | 233 | −37.736 | 70.556 | 39.144 | 1.00 | 68.97 | C |
| ATOM | 3549 | CB | VAL | A | 233 | −37.614 | 72.101 | 39.226 | 1.00 | 65.14 | C |
| ATOM | 3551 | CG1 | VAL | A | 233 | −38.495 | 72.627 | 40.323 | 1.00 | 63.75 | C |
| ATOM | 3555 | CG2 | VAL | A | 233 | −36.195 | 72.533 | 39.534 | 1.00 | 73.26 | C |
| ATOM | 3559 | C | VAL | A | 233 | −36.748 | 69.978 | 38.136 | 1.00 | 67.13 | C |
| ATOM | 3560 | O | VAL | A | 233 | −35.673 | 69.494 | 38.502 | 1.00 | 64.03 | O |
| ATOM | 3561 | N | PRO | A | 234 | −37.165 | 69.915 | 36.880 | 1.00 | 63.98 | N |
| ATOM | 3562 | CA | PRO | A | 234 | −36.322 | 69.360 | 35.826 | 1.00 | 60.78 | C |
| ATOM | 3564 | CB | PRO | A | 234 | −37.167 | 69.561 | 34.571 | 1.00 | 64.70 | C |
| ATOM | 3567 | CG | PRO | A | 234 | −38.555 | 69.542 | 35.064 | 1.00 | 59.78 | C |
| ATOM | 3570 | CD | PRO | A | 234 | −38.510 | 70.246 | 36.385 | 1.00 | 66.10 | C |
| ATOM | 3573 | C | PRO | A | 234 | −35.043 | 70.138 | 35.706 | 1.00 | 55.11 | C |
| ATOM | 3574 | O | PRO | A | 234 | −34.964 | 71.289 | 36.113 | 1.00 | 48.54 | O |
| ATOM | 3575 | N | LEU | A | 235 | −34.040 | 69.490 | 35.136 | 1.00 | 57.33 | N |
| ATOM | 3577 | CA | LEU | A | 235 | −32.711 | 70.083 | 35.029 | 1.00 | 53.95 | C |
| ATOM | 3579 | CB | LEU | A | 235 | −31.620 | 69.000 | 35.102 | 1.00 | 55.01 | C |
| ATOM | 3582 | CG | LEU | A | 235 | −30.313 | 69.123 | 34.300 | 1.00 | 51.16 | C |
| ATOM | 3584 | CD1 | LEU | A | 235 | −29.353 | 70.042 | 35.071 | 1.00 | 33.61 | C |
| ATOM | 3588 | CD2 | LEU | A | 235 | −29.681 | 67.731 | 33.987 | 1.00 | 35.49 | C |
| ATOM | 3592 | C | LEU | A | 235 | −32.708 | 70.703 | 33.659 | 1.00 | 53.02 | C |
| ATOM | 3593 | O | LEU | A | 235 | −33.311 | 70.150 | 32.717 | 1.00 | 45.91 | O |
| ATOM | 3594 | N | VAL | A | 236 | −32.028 | 71.835 | 33.557 | 1.00 | 45.82 | N |
| ATOM | 3596 | CA | VAL | A | 236 | −31.858 | 72.424 | 32.255 | 1.00 | 50.59 | C |
| ATOM | 3598 | CB | VAL | A | 236 | −32.348 | 73.874 | 32.215 | 1.00 | 54.05 | C |
| ATOM | 3600 | CG1 | VAL | A | 236 | −32.881 | 74.189 | 30.825 | 1.00 | 63.23 | C |
| ATOM | 3604 | CG2 | VAL | A | 236 | −33.449 | 74.083 | 33.228 | 1.00 | 53.03 | C |
| ATOM | 3608 | C | VAL | A | 236 | −30.403 | 72.326 | 31.853 | 1.00 | 49.70 | C |
| ATOM | 3609 | O | VAL | A | 236 | −29.514 | 72.803 | 32.565 | 1.00 | 52.45 | O |
| ATOM | 3610 | N | VAL | A | 237 | −30.187 | 71.711 | 30.696 | 1.00 | 48.09 | N |
| ATOM | 3612 | CA | VAL | A | 237 | −28.926 | 71.832 | 29.991 | 1.00 | 48.08 | C |
| ATOM | 3614 | CB | VAL | A | 237 | −28.447 | 70.446 | 29.532 | 1.00 | 52.59 | C |
| ATOM | 3616 | CG1 | VAL | A | 237 | −27.016 | 70.537 | 28.976 | 1.00 | 49.36 | C |
| ATOM | 3620 | CG2 | VAL | A | 237 | −28.534 | 69.471 | 30.694 | 1.00 | 46.48 | C |
| ATOM | 3624 | C | VAL | A | 237 | −29.035 | 72.753 | 28.788 | 1.00 | 44.28 | C |
| ATOM | 3625 | O | VAL | A | 237 | −29.851 | 72.538 | 27.895 | 1.00 | 39.05 | O |
| ATOM | 3626 | N | GLU | A | 238 | −28.194 | 73.776 | 28.771 | 1.00 | 43.39 | N |
| ATOM | 3628 | CA | GLU | A | 238 | −28.326 | 74.854 | 27.806 | 1.00 | 47.13 | C |
| ATOM | 3630 | CB | GLU | A | 238 | −28.683 | 76.148 | 28.536 | 1.00 | 56.83 | C |
| ATOM | 3633 | CG | GLU | A | 238 | −28.701 | 77.375 | 27.630 | 1.00 | 62.22 | C |
| ATOM | 3636 | CD | GLU | A | 238 | −29.108 | 78.640 | 28.352 | 1.00 | 68.97 | C |
| ATOM | 3637 | OE1 | GLU | A | 238 | −28.674 | 78.823 | 29.513 | 1.00 | 73.54 | O |
| ATOM | 3638 | OE2 | GLU | A | 238 | −29.866 | 79.428 | 27.742 | 1.00 | 76.38 | O |
| ATOM | 3639 | C | GLU | A | 238 | −27.056 | 75.091 | 26.986 | 1.00 | 49.39 | C |
| ATOM | 3640 | O | GLU | A | 238 | −25.959 | 75.334 | 27.520 | 1.00 | 40.33 | O |
| ATOM | 3641 | N | TYR | A | 239 | −27.239 | 75.103 | 25.670 | 1.00 | 47.11 | N |
| ATOM | 3643 | CA | TYR | A | 239 | −26.183 | 75.489 | 24.743 | 1.00 | 43.91 | C |
| ATOM | 3645 | CB | TYR | A | 239 | −26.137 | 74.384 | 23.683 | 1.00 | 36.93 | C |
| ATOM | 3648 | CG | TYR | A | 239 | −25.763 | 73.059 | 24.276 | 1.00 | 41.80 | C |
| ATOM | 3649 | CD1 | TYR | A | 239 | −26.721 | 72.166 | 24.708 | 1.00 | 38.13 | C |
| ATOM | 3651 | CE1 | TYR | A | 239 | −26.356 | 70.991 | 25.360 | 1.00 | 28.37 | C |
| ATOM | 3653 | CZ | TYR | A | 239 | −25.018 | 70.707 | 25.598 | 1.00 | 32.48 | C |
| ATOM | 3654 | OH | TYR | A | 239 | −24.604 | 69.533 | 26.212 | 1.00 | 41.99 | O |
| ATOM | 3656 | CE2 | TYR | A | 239 | −24.067 | 71.608 | 25.210 | 1.00 | 32.04 | C |
| ATOM | 3658 | CD2 | TYR | A | 239 | −24.442 | 72.771 | 24.565 | 1.00 | 39.27 | C |
| ATOM | 3660 | C | TYR | A | 239 | −26.529 | 76.869 | 24.142 | 1.00 | 46.97 | C |
| ATOM | 3661 | O | TYR | A | 239 | −27.602 | 77.009 | 23.566 | 1.00 | 51.30 | O |
| ATOM | 3662 | N | LYS | A | 240 | −25.664 | 77.885 | 24.205 | 1.00 | 49.68 | N |
| ATOM | 3664 | CA | LYS | A | 240 | −25.950 | 79.095 | 23.411 | 1.00 | 51.95 | C |
| ATOM | 3666 | CB | LYS | A | 240 | −25.254 | 80.386 | 23.893 | 1.00 | 54.77 | C |
| ATOM | 3669 | CG | LYS | A | 240 | −26.025 | 81.150 | 25.027 | 1.00 | 85.05 | C |
| ATOM | 3672 | CD | LYS | A | 240 | −26.820 | 82.431 | 24.573 | 1.00 | 111.95 | C |
| ATOM | 3675 | CE | LYS | A | 240 | −27.864 | 82.964 | 25.618 | 1.00 | 109.66 | C |
| ATOM | 3678 | NZ | LYS | A | 240 | −29.305 | 83.111 | 25.158 | 1.00 | 79.60 | N |
| ATOM | 3682 | C | LYS | A | 240 | −25.621 | 78.838 | 21.956 | 1.00 | 43.11 | C |
| ATOM | 3683 | O | LYS | A | 240 | −24.695 | 78.111 | 21.641 | 1.00 | 48.44 | O |
| ATOM | 3684 | N | ILE | A | 241 | −26.439 | 79.374 | 21.063 | 1.00 | 45.78 | N |
| ATOM | 3686 | CA | ILE | A | 241 | −26.211 | 79.249 | 19.624 | 1.00 | 43.09 | C |
| ATOM | 3688 | CB | ILE | A | 241 | −27.493 | 78.822 | 18.902 | 1.00 | 39.80 | C |
| ATOM | 3690 | CG1 | ILE | A | 241 | −28.114 | 77.570 | 19.502 | 1.00 | 45.72 | C |
| ATOM | 3693 | CD1 | ILE | A | 241 | −29.343 | 77.089 | 18.738 | 1.00 | 40.54 | C |
| ATOM | 3697 | CG2 | ILE | A | 241 | −27.247 | 78.572 | 17.454 | 1.00 | 44.94 | C |
| ATOM | 3701 | C | ILE | A | 241 | −25.890 | 80.671 | 19.194 | 1.00 | 51.02 | C |
| ATOM | 3702 | O | ILE | A | 241 | −26.782 | 81.535 | 19.140 | 1.00 | 47.93 | O |
| ATOM | 3703 | N | ALA | A | 242 | −24.605 | 80.933 | 18.965 | 1.00 | 56.93 | N |
| ATOM | 3705 | CA | ALA | A | 242 | −24.045 | 82.248 | 19.255 | 1.00 | 55.79 | C |
| ATOM | 3707 | CB | ALA | A | 242 | −22.561 | 82.247 | 19.061 | 1.00 | 59.59 | C |
| ATOM | 3711 | C | ALA | A | 242 | −24.687 | 83.152 | 18.252 | 1.00 | 56.99 | C |
| ATOM | 3712 | O | ALA | A | 242 | −24.783 | 82.770 | 17.077 | 1.00 | 47.10 | O |
| ATOM | 3713 | N | ASP | A | 243 | −25.168 | 84.295 | 18.742 | 1.00 | 62.76 | N |
| ATOM | 3715 | CA | ASP | A | 243 | −25.794 | 85.321 | 17.896 | 1.00 | 68.37 | C |
| ATOM | 3717 | CB | ASP | A | 243 | −24.995 | 85.567 | 16.594 | 1.00 | 68.46 | C |
| ATOM | 3720 | CG | ASP | A | 243 | −23.807 | 86.507 | 16.774 | 1.00 | 77.36 | C |
| ATOM | 3721 | OD1 | ASP | A | 243 | −23.172 | 86.532 | 17.857 | 1.00 | 85.26 | O |
| ATOM | 3722 | OD2 | ASP | A | 243 | −23.416 | 87.241 | 15.842 | 1.00 | 93.50 | O |
| ATOM | 3723 | C | ASP | A | 243 | −27.203 | 84.987 | 17.438 | 1.00 | 61.97 | C |
| ATOM | 3724 | O | ASP | A | 243 | −27.692 | 85.696 | 16.576 | 1.00 | 69.55 | O |
| ATOM | 3725 | N | MET | A | 244 | −27.792 | 83.862 | 17.828 | 1.00 | 51.89 | N |
| ATOM | 3727 | CA | MET | A | 244 | −29.100 | 83.512 | 17.265 | 1.00 | 54.43 | C |
| ATOM | 3729 | CB | MET | A | 244 | −29.002 | 82.359 | 16.268 | 1.00 | 52.20 | C |
| ATOM | 3732 | CG | MET | A | 244 | −28.336 | 82.684 | 14.992 | 1.00 | 53.56 | C |
| ATOM | 3735 | SD | MET | A | 244 | −28.893 | 81.511 | 13.832 | 1.00 | 60.80 | S |
| ATOM | 3736 | CE | MET | A | 244 | −30.376 | 82.263 | 13.300 | 1.00 | 65.06 | C |
| ATOM | 3740 | C | MET | A | 244 | −30.155 | 83.103 | 18.287 | 1.00 | 49.30 | C |
| ATOM | 3741 | O | MET | A | 244 | −31.339 | 83.155 | 18.021 | 1.00 | 46.04 | O |
| ATOM | 3742 | N | GLY | A | 245 | −29.728 | 82.573 | 19.415 | 1.00 | 50.47 | N |
| ATOM | 3744 | CA | GLY | A | 245 | −30.638 | 81.848 | 20.283 | 1.00 | 54.40 | C |
| ATOM | 3747 | C | GLY | A | 245 | −29.939 | 80.821 | 21.162 | 1.00 | 50.46 | C |
| ATOM | 3748 | O | GLY | A | 245 | −28.783 | 80.992 | 21.583 | 1.00 | 44.75 | O |
| ATOM | 3749 | N | HIS | A | 246 | −30.655 | 79.730 | 21.403 | 1.00 | 41.88 | N |
| ATOM | 3751 | CA | HIS | A | 246 | −30.166 | 78.701 | 22.303 | 1.00 | 50.23 | C |
| ATOM | 3753 | CB | HIS | A | 246 | −30.198 | 79.130 | 23.784 | 1.00 | 46.63 | C |
| ATOM | 3756 | CG | HIS | A | 246 | −31.494 | 79.726 | 24.211 | 1.00 | 62.95 | C |
| ATOM | 3757 | ND1 | HIS | A | 246 | −31.810 | 81.048 | 23.979 | 1.00 | 80.32 | N |
| ATOM | 3759 | CE1 | HIS | A | 246 | −33.038 | 81.278 | 24.406 | 1.00 | 90.00 | C |
| ATOM | 3761 | NE2 | HIS | A | 246 | −33.538 | 80.147 | 24.878 | 1.00 | 80.54 | N |
| ATOM | 3763 | CD2 | HIS | A | 246 | −32.591 | 79.160 | 24.763 | 1.00 | 77.06 | C |
| ATOM | 3765 | C | HIS | A | 246 | −30.971 | 77.424 | 22.133 | 1.00 | 50.13 | C |
| ATOM | 3766 | O | HIS | A | 246 | −32.089 | 77.422 | 21.619 | 1.00 | 46.91 | O |
| ATOM | 3767 | N | LEU | A | 247 | −30.361 | 76.337 | 22.579 | 1.00 | 40.32 | N |
| ATOM | 3769 | CA | LEU | A | 247 | −31.038 | 75.077 | 22.652 | 1.00 | 35.67 | C |
| ATOM | 3771 | CB | LEU | A | 247 | −30.257 | 74.097 | 21.806 | 1.00 | 41.86 | C |
| ATOM | 3774 | CG | LEU | A | 247 | −30.899 | 72.795 | 21.383 | 1.00 | 43.71 | C |
| ATOM | 3776 | CD1 | LEU | A | 247 | −31.985 | 73.075 | 20.376 | 1.00 | 49.67 | C |
| ATOM | 3780 | CD2 | LEU | A | 247 | −29.788 | 71.998 | 20.735 | 1.00 | 59.25 | C |
| ATOM | 3784 | C | LEU | A | 247 | −31.009 | 74.704 | 24.118 | 1.00 | 35.56 | C |
| ATOM | 3785 | O | LEU | A | 247 | −29.957 | 74.747 | 24.745 | 1.00 | 40.40 | O |
| ATOM | 3786 | N | LYS | A | 248 | −32.176 | 74.442 | 24.693 | 1.00 | 41.77 | N |
| ATOM | 3788 | CA | LYS | A | 248 | −32.286 | 74.052 | 26.100 | 1.00 | 40.89 | C |
| ATOM | 3790 | CB | LYS | A | 248 | −33.175 | 75.028 | 26.857 | 1.00 | 41.91 | C |
| ATOM | 3793 | CG | LYS | A | 248 | −32.528 | 76.375 | 27.262 | 1.00 | 48.39 | C |
| ATOM | 3796 | CD | LYS | A | 248 | −33.585 | 77.389 | 27.843 | 1.00 | 49.02 | C |
| ATOM | 3799 | CE | LYS | A | 248 | −33.042 | 78.834 | 27.952 | 1.00 | 45.75 | C |
| ATOM | 3802 | NZ | LYS | A | 248 | −33.412 | 79.555 | 29.196 | 1.00 | 59.65 | N |
| ATOM | 3806 | C | LYS | A | 248 | −32.880 | 72.639 | 26.191 | 1.00 | 45.04 | C |
| ATOM | 3807 | O | LYS | A | 248 | −34.000 | 72.383 | 25.725 | 1.00 | 40.31 | O |
| ATOM | 3808 | N | TYR | A | 249 | −32.105 | 71.702 | 26.725 | 1.00 | 41.74 | N |
| ATOM | 3810 | CA | TYR | A | 249 | −32.693 | 70.434 | 27.085 | 1.00 | 43.77 | C |
| ATOM | 3812 | CB | TYR | A | 249 | −31.699 | 69.310 | 26.899 | 1.00 | 48.94 | C |
| ATOM | 3815 | CG | TYR | A | 249 | −31.195 | 69.183 | 25.479 | 1.00 | 49.32 | C |
| ATOM | 3816 | CD1 | TYR | A | 249 | −31.751 | 68.269 | 24.611 | 1.00 | 30.59 | C |
| ATOM | 3818 | CE1 | TYR | A | 249 | −31.273 | 68.135 | 23.316 | 1.00 | 33.27 | C |
| ATOM | 3820 | CZ | TYR | A | 249 | −30.207 | 68.891 | 22.893 | 1.00 | 39.05 | C |
| ATOM | 3821 | OH | TYR | A | 249 | −29.753 | 68.784 | 21.605 | 1.00 | 45.16 | O |
| ATOM | 3823 | CE2 | TYR | A | 249 | −29.642 | 69.804 | 23.736 | 1.00 | 40.89 | C |
| ATOM | 3825 | CD2 | TYR | A | 249 | −30.142 | 69.955 | 25.017 | 1.00 | 40.61 | C |
| ATOM | 3827 | C | TYR | A | 249 | −33.138 | 70.479 | 28.529 | 1.00 | 48.24 | C |
| ATOM | 3828 | O | TYR | A | 249 | −32.421 | 71.006 | 29.385 | 1.00 | 44.56 | O |
| ATOM | 3829 | N | TYR | A | 250 | −34.334 | 69.928 | 28.754 | 1.00 | 51.75 | N |
| ATOM | 3831 | CA | TYR | A | 250 | −34.948 | 69.785 | 30.069 | 1.00 | 50.38 | C |
| ATOM | 3833 | CB | TYR | A | 250 | −36.316 | 70.462 | 30.093 | 1.00 | 52.59 | C |
| ATOM | 3836 | CG | TYR | A | 250 | −36.254 | 71.977 | 30.108 | 1.00 | 44.63 | C |
| ATOM | 3837 | CD1 | TYR | A | 250 | −36.345 | 72.683 | 28.939 | 1.00 | 42.22 | C |
| ATOM | 3839 | CE1 | TYR | A | 250 | −36.341 | 74.049 | 28.931 | 1.00 | 52.03 | C |
| ATOM | 3841 | CZ | TYR | A | 250 | −36.252 | 74.745 | 30.085 | 1.00 | 47.80 | C |
| ATOM | 3842 | OH | TYR | A | 250 | −36.247 | 76.110 | 29.980 | 1.00 | 62.97 | O |
| ATOM | 3844 | CE2 | TYR | A | 250 | −36.211 | 74.077 | 31.278 | 1.00 | 61.03 | C |
| ATOM | 3846 | CD2 | TYR | A | 250 | −36.217 | 72.693 | 31.286 | 1.00 | 47.73 | C |
| ATOM | 3848 | C | TYR | A | 250 | −35.167 | 68.329 | 30.416 | 1.00 | 47.71 | C |
| ATOM | 3849 | O | TYR | A | 250 | −35.647 | 67.557 | 29.581 | 1.00 | 49.16 | O |
| ATOM | 3850 | N | LEU | A | 251 | −34.842 | 67.972 | 31.660 | 1.00 | 51.56 | N |
| ATOM | 3852 | CA | LEU | A | 251 | −34.817 | 66.558 | 32.082 | 1.00 | 47.05 | C |
| ATOM | 3854 | CB | LEU | A | 251 | −33.405 | 65.978 | 32.074 | 1.00 | 42.70 | C |
| ATOM | 3857 | CG | LEU | A | 251 | −33.214 | 64.508 | 32.467 | 1.00 | 33.92 | C |
| ATOM | 3859 | CD1 | LEU | A | 251 | −33.942 | 63.536 | 31.567 | 1.00 | 55.55 | C |
| ATOM | 3863 | CD2 | LEU | A | 251 | −31.755 | 64.122 | 32.485 | 1.00 | 35.13 | C |
| ATOM | 3867 | C | LEU | A | 251 | −35.398 | 66.320 | 33.461 | 1.00 | 48.75 | C |
| ATOM | 3868 | O | LEU | A | 251 | −35.011 | 66.934 | 34.473 | 1.00 | 44.03 | O |
| ATOM | 3869 | N | ALA | A | 252 | −36.308 | 65.354 | 33.473 | 1.00 | 46.78 | N |
| ATOM | 3871 | CA | ALA | A | 252 | −37.070 | 65.059 | 34.661 | 1.00 | 41.23 | C |
| ATOM | 3873 | CB | ALA | A | 252 | −38.331 | 64.310 | 34.253 | 1.00 | 47.07 | C |
| ATOM | 3877 | C | ALA | A | 252 | −36.177 | 64.188 | 35.513 | 1.00 | 47.71 | C |
| ATOM | 3878 | O | ALA | A | 252 | −35.566 | 63.231 | 35.031 | 1.00 | 50.77 | O |
| ATOM | 3879 | N | PRO | A | 253 | −36.076 | 64.518 | 36.786 | 1.00 | 50.91 | N |
| ATOM | 3800 | CA | PRO | A | 253 | −35.183 | 63.783 | 37.678 | 1.00 | 49.87 | C |
| ATOM | 3882 | CB | PRO | A | 253 | −35.159 | 64.637 | 38.920 | 1.00 | 48.01 | C |
| ATOM | 3885 | CG | PRO | A | 253 | −36.511 | 65.233 | 38.921 | 1.00 | 54.82 | C |
| ATOM | 3888 | CD | PRO | A | 253 | −36.812 | 65.574 | 37.487 | 1.00 | 56.91 | C |
| ATOM | 3891 | C | PRO | A | 253 | −35.767 | 62.421 | 38.004 | 1.00 | 55.86 | C |
| ATOM | 3892 | O | PRO | A | 253 | −36.969 | 62.190 | 37.789 | 1.00 | 47.88 | O |
| ATOM | 3893 | N | LYS | A | 254 | −34.862 | 61.551 | 38.468 | 1.00 | 58.95 | N |
| ATOM | 3895 | CA | LYS | A | 254 | −35.150 | 60.330 | 39.217 | 1.00 | 58.55 | C |
| ATOM | 3897 | CB | LYS | A | 254 | −34.064 | 59.300 | 38.924 | 1.00 | 58.21 | C |
| ATOM | 3900 | CG | LYS | A | 254 | −34.236 | 58.614 | 37.567 | 1.00 | 72.74 | C |
| ATOM | 3903 | CD | LYS | A | 254 | −33.163 | 57.574 | 37.278 | 1.00 | 74.82 | C |
| ATOM | 3906 | CE | LYS | A | 254 | −33.729 | 56.226 | 36.851 | 1.00 | 69.95 | C |
| ATOM | 3909 | NZ | LYS | A | 254 | −32.717 | 55.174 | 37.201 | 1.00 | 69.55 | N |
| ATOM | 3913 | C | LYS | A | 254 | −35.237 | 60.548 | 40.725 | 1.00 | 62.43 | C |
| ATOM | 3914 | O | LYS | A | 254 | −34.404 | 61.245 | 41.353 | 1.00 | 48.97 | O |
| ATOM | 3915 | N | ILE | A | 255 | −36.281 | 59.944 | 41.290 | 1.00 | 71.60 | N |
| ATOM | 3917 | CA | ILE | A | 255 | −36.770 | 60.329 | 42.614 | 1.00 | 80.41 | C |
| ATOM | 3919 | CB | ILE | A | 255 | −37.680 | 61.596 | 42.444 | 1.00 | 79.11 | C |
| ATOM | 3921 | CG1 | ILE | A | 255 | −37.074 | 62.765 | 43.223 | 1.00 | 92.92 | C |
| ATOM | 3924 | CD1 | ILE | A | 255 | −36.134 | 62.348 | 44.408 | 1.00 | 101.86 | C |
| ATOM | 3928 | CG2 | ILE | A | 255 | −39.159 | 61.329 | 42.777 | 1.00 | 76.89 | C |
| ATOM | 3932 | C | ILE | A | 255 | −37.414 | 59.130 | 43.340 | 1.00 | 81.92 | C |
| ATOM | 3933 | O | ILE | A | 255 | −37.949 | 58.244 | 42.687 | 1.00 | 75.70 | O |
| ATOM | 3934 | N | GLU | A | 256 | −37.297 | 59.037 | 44.665 | 1.00 | 97.97 | N |
| ATOM | 3936 | CA | GLU | A | 256 | −38.164 | 58.133 | 45.454 | 1.00 | 108.24 | C |
| ATOM | 3938 | CB | GLU | A | 256 | −37.433 | 56.839 | 45.852 | 1.00 | 109.21 | C |
| ATOM | 3941 | CG | GLU | A | 256 | −36.985 | 56.760 | 47.314 | 1.00 | 115.31 | C |
| ATOM | 3944 | CD | GLU | A | 256 | −35.513 | 57.119 | 47.545 | 1.00 | 120.15 | C |
| ATOM | 3945 | OE1 | GLU | A | 256 | −34.647 | 56.210 | 47.501 | 1.00 | 120.17 | O |
| ATOM | 3946 | OE2 | GLU | A | 256 | −35.211 | 58.308 | 47.809 | 1.00 | 107.04 | O |
| ATOM | 3947 | C | GLU | A | 256 | −38.776 | 58.769 | 46.710 | 1.00 | 117.14 | C |
| ATOM | 3948 | O | GLU | A | 256 | −39.875 | 58.373 | 47.120 | 1.00 | 122.98 | O |
| ATOM | 3949 | N | ASP | A | 257 | −38.047 | 59.711 | 47.323 | 1.00 | 123.78 | N |
| ATOM | 3951 | CA | ASP | A | 287 | −38.549 | 60.589 | 48.397 | 1.00 | 125.10 | C |
| ATOM | 3953 | CB | ASP | A | 257 | −39.809 | 61.353 | 47.960 | 1.00 | 125.53 | C |
| ATOM | 3956 | CG | ASP | A | 257 | −39.491 | 62.741 | 47.410 | 1.00 | 121.84 | C |
| ATOM | 3957 | OD1 | ASP | A | 257 | −38.382 | 62.939 | 46.855 | 1.00 | 91.45 | O |
| ATOM | 3958 | OD2 | ASP | A | 257 | −40.312 | 63.695 | 47.487 | 1.00 | 128.84 | O |
| ATOM | 3959 | C | ASP | A | 257 | −38.826 | 59.886 | 49.730 | 1.00 | 126.09 | C |
| ATOM | 3960 | O | ASP | A | 257 | −39.210 | 60.528 | 50.714 | 1.00 | 125.12 | O |
| ATOM | 3961 | N | MET | C | 1 | 6.537 | 33.339 | −20.719 | 1.00 | 62.05 | N |
| ATOM | 3963 | CA | MET | C | 1 | 6.498 | 33.899 | −19.316 | 1.00 | 61.71 | C |
| ATOM | 3965 | CB | MET | C | 1 | 7.886 | 33.735 | −18.708 | 1.00 | 60.10 | C |
| ATOM | 3968 | CG | MET | C | 1 | 8.365 | 34.929 | −17.955 | 1.00 | 66.53 | C |
| ATOM | 3971 | SD | MET | C | 1 | 8.167 | 34.723 | −16.166 | 1.00 | 101.30 | S |
| ATOM | 3972 | CE | MET | C | 1 | 9.839 | 35.082 | −15.541 | 1.00 | 66.56 | C |
| ATOM | 3976 | C | MET | C | 1 | 6.053 | 35.372 | −19.229 | 1.00 | 55.91 | C |
| ATOM | 3977 | O | MET | C | 1 | 6.324 | 36.166 | −20.133 | 1.00 | 56.97 | O |
| ATOM | 3980 | N | PHE | C | 2 | 5.367 | 35.745 | −18.152 | 1.00 | 52.00 | N |
| ATOM | 3982 | CA | PHE | C | 2 | 4.801 | 37.102 | −18.025 | 1.00 | 46.24 | C |
| ATOM | 3984 | CB | PHE | C | 2 | 3.270 | 37.070 | −17.825 | 1.00 | 49.18 | C |
| ATOM | 3987 | CG | PHE | C | 2 | 2.671 | 38.356 | −17.274 | 1.00 | 35.44 | C |
| ATOM | 3988 | CD1 | PHE | C | 2 | 2.761 | 39.545 | −17.951 | 1.00 | 38.92 | C |
| ATOM | 3990 | CE1 | PHE | C | 2 | 2.172 | 40.717 | −17.431 | 1.00 | 32.26 | C |
| ATOM | 3992 | CZ | PHE | C | 2 | 1.515 | 40.688 | −16.250 | 1.00 | 36.98 | C |
| ATOM | 3994 | CE2 | PHE | C | 2 | 1.370 | 39.502 | −15.596 | 1.00 | 43.40 | C |
| ATOM | 3996 | CD2 | PHE | C | 2 | 1.967 | 38.356 | −16.091 | 1.00 | 43.70 | C |
| ATOM | 3998 | C | PHE | C | 2 | 5.429 | 37.803 | −16.837 | 1.00 | 43.42 | C |
| ATOM | 3999 | O | PHE | C | 2 | 5.511 | 37.251 | −15.745 | 1.00 | 35.20 | O |
| ATOM | 4000 | N | GLU | C | 3 | 5.860 | 39.037 | −17.030 | 1.00 | 44.02 | N |
| ATOM | 4002 | CA | GLU | C | 3 | 6.485 | 39.728 | −15.920 | 1.00 | 46.06 | C |
| ATOM | 4004 | CB | GLU | C | 3 | 7.973 | 39.361 | −15.816 | 1.00 | 48.80 | C |
| ATOM | 4007 | CG | GLU | C | 3 | 8.565 | 39.715 | −14.464 | 1.00 | 49.67 | C |
| ATOM | 4010 | CD | GLU | C | 3 | 10.016 | 39.323 | −14.264 | 1.00 | 42.71 | C |
| ATOM | 4011 | OE1 | GLU | C | 3 | 10.913 | 39.846 | −14.955 | 1.00 | 77.68 | O |
| ATOM | 4012 | OE2 | GLU | C | 3 | 10.278 | 38.595 | −13.289 | 1.00 | 62.46 | O |
| ATOM | 4013 | C | GLU | C | 3 | 6.303 | 41.224 | −16.064 | 1.00 | 43.47 | C |
| ATOM | 4014 | O | GLU | C | 3 | 6.791 | 41.848 | −17.003 | 1.00 | 54.40 | O |
| ATOM | 4015 | N | ALA | C | 4 | 5.634 | 41.821 | −15.098 | 1.00 | 37.77 | N |
| ATOM | 4017 | CA | ALA | C | 4 | 5.358 | 43.230 | −15.233 | 1.00 | 41.12 | C |
| ATOM | 4019 | CB | ALA | C | 4 | 3.887 | 43.439 | −15.475 | 1.00 | 47.01 | C |
| ATOM | 4023 | C | ALA | C | 4 | 5.766 | 43.850 | −13.926 | 1.00 | 42.42 | C |
| ATOM | 4024 | O | ALA | C | 4 | 5.288 | 43.399 | −12.871 | 1.00 | 40.97 | O |
| ATOM | 4025 | N | ARG | C | 5 | 6.665 | 44.834 | −13.985 | 1.00 | 32.34 | N |
| ATOM | 4027 | CA | ARG | C | 5 | 7.070 | 45.490 | −12.760 | 1.00 | 35.03 | C |
| ATOM | 4029 | CB | ARG | C | 5 | 8.584 | 45.514 | −12.666 | 1.00 | 42.57 | C |
| ATOM | 4032 | CG | ARG | C | 5 | 9.214 | 46.327 | −11.517 | 1.00 | 43.56 | C |
| ATOM | 4035 | CD | ARG | C | 5 | 10.761 | 46.252 | −11.575 | 1.00 | 53.82 | C |
| ATOM | 4038 | NE | ARG | C | 5 | 11.491 | 47.375 | −11.002 | 1.00 | 39.94 | N |
| ATOM | 4040 | CZ | ARG | C | 5 | 12.071 | 47.366 | −9.802 | 1.00 | 64.98 | C |
| ATOM | 4041 | NH1 | ARG | C | 5 | 12.027 | 46.304 | −8.991 | 1.00 | 58.35 | N |
| ATOM | 4044 | NH2 | ARG | C | 5 | 12.679 | 48.467 | −9.382 | 1.00 | 78.35 | N |
| ATOM | 4047 | C | ARG | C | 5 | 6.507 | 46.900 | −12.690 | 1.00 | 37.78 | C |
| ATOM | 4048 | O | ARG | C | 5 | 6.676 | 47.678 | −13.623 | 1.00 | 35.01 | O |
| ATOM | 4049 | N | LEU | C | 6 | 5.865 | 47.220 | −11.566 | 1.00 | 36.87 | N |
| ATOM | 4051 | CA | LEU | C | 6 | 5.276 | 48.526 | −11.365 | 1.00 | 31.63 | C |
| ATOM | 4053 | CB | LEU | C | 6 | 3.775 | 48.409 | −11.179 | 1.00 | 37.67 | C |
| ATOM | 4056 | CG | LEU | C | 6 | 2.911 | 49.660 | −11.399 | 1.00 | 32.73 | C |
| ATOM | 4058 | CD1 | LEU | C | 6 | 2.876 | 50.093 | −12.837 | 1.00 | 46.11 | C |
| ATOM | 4062 | CD2 | LEU | C | 6 | 1.529 | 49.268 | −11.024 | 1.00 | 51.06 | C |
| ATOM | 4066 | C | LEU | C | 6 | 5.856 | 49.252 | −10.185 | 1.00 | 35.21 | C |
| ATOM | 4067 | O | LEU | C | 6 | 5.731 | 48.790 | −9.058 | 1.00 | 39.05 | O |
| ATOM | 4068 | N | VAL | C | 7 | 6.459 | 50.408 | −10.458 | 1.00 | 39.59 | N |
| ATOM | 4070 | CA | VAL | C | 7 | 7.140 | 51.209 | −9.435 | 1.00 | 38.59 | C |
| ATOM | 4072 | CB | VAL | C | 7 | 7.969 | 52.383 | −10.034 | 1.00 | 32.41 | C |
| ATOM | 4074 | CG1 | VAL | C | 7 | 8.543 | 53.233 | −8.954 | 1.00 | 39.14 | C |
| ATOM | 4078 | CG2 | VAL | C | 7 | 9.058 | 51.864 | −10.890 | 1.00 | 37.52 | C |
| ATOM | 4082 | C | VAL | C | 7 | 6.139 | 51.822 | −8.476 | 1.00 | 31.88 | C |
| ATOM | 4083 | O | VAL | C | 7 | 6.309 | 51.703 | −7.266 | 1.00 | 38.01 | O |
| ATOM | 4084 | N | GLN | C | 8 | 5.150 | 52.537 | −9.004 | 1.00 | 36.16 | N |
| ATOM | 4086 | CA | GLN | C | 8 | 4.049 | 53.009 | −8.165 | 1.00 | 42.14 | C |
| ATOM | 4088 | CB | GLN | C | 8 | 3.279 | 54.173 | −8.754 | 1.00 | 43.24 | C |
| ATOM | 4091 | CG | GLN | C | 8 | 3.777 | 55.549 | −8.326 | 1.00 | 68.88 | C |
| ATOM | 4094 | CD | GLN | C | 8 | 3.628 | 56.502 | −9.500 | 1.00 | 89.60 | C |
| ATOM | 4095 | OE1 | GLN | C | 8 | 2.499 | 56.829 | −9.857 | 1.00 | 85.74 | O |
| ATOM | 4096 | NE2 | GLN | C | 8 | 4.731 | 56.821 | −10.186 | 1.00 | 76.93 | N |
| ATOM | 4099 | C | GLN | C | 8 | 3.032 | 51.929 | −7.907 | 1.00 | 35.38 | C |
| ATOM | 4100 | O | GLN | C | 8 | 1.976 | 51.945 | −8.501 | 1.00 | 38.26 | O |
| ATOM | 4101 | N | GLY | C | 9 | 3.323 | 51.097 | −6.916 | 1.00 | 34.54 | N |
| ATOM | 4103 | CA | GLY | C | 9 | 2.464 | 50.007 | −6.518 | 1.00 | 40.86 | C |
| ATOM | 4106 | C | GLY | C | 9 | 1.062 | 50.429 | −6.136 | 1.00 | 41.06 | C |
| ATOM | 4107 | O | GLY | C | 9 | 0.135 | 49.648 | −6.278 | 1.00 | 37.39 | O |
| ATOM | 4108 | N | SER | C | 10 | 0.942 | 51.640 | −5.606 | 1.00 | 48.18 | N |
| ATOM | 4110 | CA | SER | C | 10 | −0.337 | 52.204 | −5.202 | 1.00 | 45.42 | C |
| ATOM | 4112 | CB | SER | C | 10 | −0.227 | 53.714 | −4.934 | 1.00 | 49.22 | C |
| ATOM | 4115 | OG | SER | C | 10 | −0.332 | 54.471 | −6.145 | 1.00 | 54.16 | O |
| ATOM | 4117 | C | SER | C | 10 | −1.324 | 51.993 | −6.321 | 1.00 | 40.89 | C |
| ATOM | 4118 | O | SER | C | 10 | −2.497 | 51.774 | −6.042 | 1.00 | 19.70 | O |
| ATOM | 4119 | N | ILE | C | 11 | −0.867 | 52.125 | −7.570 | 1.00 | 39.63 | N |
| ATOM | 4121 | CA | ILE | C | 11 | −1.817 | 52.207 | −8.665 | 1.00 | 39.03 | C |
| ATOM | 4123 | CB | ILE | C | 11 | −1.117 | 52.303 | −10.005 | 1.00 | 38.43 | C |
| ATOM | 4125 | CG1 | ILE | C | 11 | −0.682 | 53.714 | −10.330 | 1.00 | 31.28 | C |
| ATOM | 4128 | CD1 | ILE | C | 11 | 0.281 | 53.668 | −11.485 | 1.00 | 43.37 | C |
| ATOM | 4132 | CG2 | ILE | C | 11 | −2.065 | 51.893 | −11.100 | 1.00 | 47.54 | C |
| ATOM | 4136 | C | ILE | C | 11 | −2.581 | 50.893 | −8.670 | 1.00 | 42.63 | C |
| ATOM | 4137 | O | ILE | C | 11 | −3.782 | 50.884 | −8.854 | 1.00 | 41.68 | O |
| ATOM | 4138 | N | LEU | C | 12 | −1.874 | 49.779 | −8.514 | 1.00 | 37.29 | N |
| ATOM | 4140 | CA | LEU | C | 12 | −2.517 | 48.488 | −8.587 | 1.00 | 36.44 | C |
| ATOM | 4142 | CB | LEU | C | 12 | −1.443 | 47.428 | −8.607 | 1.00 | 40.60 | C |
| ATOM | 4145 | CG | LEU | C | 12 | −1.900 | 46.173 | −9.338 | 1.00 | 51.37 | C |
| ATOM | 4147 | CD1 | LEU | C | 12 | −2.169 | 46.518 | −10.794 | 1.00 | 58.76 | C |
| ATOM | 4151 | CD2 | LEU | C | 12 | −0.826 | 45.092 | −9.249 | 1.00 | 50.26 | C |
| ATOM | 4155 | C | LEU | C | 12 | −3.481 | 48.244 | −7.426 | 1.00 | 38.85 | C |
| ATOM | 4156 | O | LEU | C | 12 | −4.524 | 47.567 | −7.527 | 1.00 | 32.33 | O |
| ATOM | 4157 | N | LYS | C | 13 | −3.083 | 48.805 | −6.294 | 1.00 | 39.27 | N |
| ATOM | 4159 | CA | LYS | C | 13 | −3.822 | 48.668 | −5.058 | 1.00 | 37.62 | C |
| ATOM | 4161 | CB | LYS | C | 13 | −3.059 | 49.343 | −3.922 | 1.00 | 36.23 | C |
| ATOM | 4164 | CG | LYS | C | 13 | −2.247 | 48.422 | −3.024 | 1.00 | 52.67 | C |
| ATOM | 4167 | CD | LYS | C | 13 | −1.166 | 49.216 | −2.290 | 1.00 | 54.96 | C |
| ATOM | 4170 | CE | LYS | C | 13 | −1.524 | 49.539 | −0.849 | 1.00 | 47.68 | C |
| ATOM | 4173 | NZ | LYS | C | 13 | −2.276 | 48.360 | −0.352 | 1.00 | 47.39 | N |
| ATOM | 4177 | C | LYS | C | 13 | −5.133 | 49.421 | −5.282 | 1.00 | 41.92 | C |
| ATOM | 4178 | O | LYS | C | 13 | −6.224 | 48.911 | −4.989 | 1.00 | 36.22 | O |
| ATOM | 4179 | N | LYS | C | 14 | −5.049 | 50.647 | −5.796 | 1.00 | 33.99 | N |
| ATOM | 4181 | CA | LYS | C | 14 | −6.284 | 51.364 | −5.882 | 1.00 | 30.13 | C |
| ATOM | 4183 | CB | LYS | C | 14 | −6.209 | 52.896 | −5.873 | 1.00 | 31.38 | C |
| ATOM | 4186 | CG | LYS | C | 14 | −4.917 | 53.670 | −5.937 | 1.00 | 46.67 | C |
| ATOM | 4189 | CD | LYS | C | 14 | −5.119 | 55.087 | −5.312 | 1.00 | 43.33 | C |
| ATOM | 4192 | CE | LYS | C | 14 | −3.826 | 55.889 | −5.129 | 1.00 | 33.56 | C |
| ATOM | 4195 | NZ | LYS | C | 14 | −2.997 | 56.160 | −6.435 | 1.00 | 48.74 | N |
| ATOM | 4199 | C | LYS | C | 14 | −7.185 | 50.820 | −6.970 | 1.00 | 32.87 | C |
| ATOM | 4200 | O | LYS | C | 14 | −8.412 | 50.892 | −6.853 | 1.00 | 34.65 | O |
| ATOM | 4201 | N | VAL | C | 15 | −6.581 | 50.202 | −7.978 | 1.00 | 33.32 | N |
| ATOM | 4203 | CA | VAL | C | 15 | −7.345 | 49.671 | −9.101 | 1.00 | 33.35 | C |
| ATOM | 4205 | CB | VAL | C | 15 | −6.414 | 49.126 | −10.229 | 1.00 | 31.07 | C |
| ATOM | 4207 | CG1 | VAL | C | 15 | −7.136 | 48.164 | −11.174 | 1.00 | 25.71 | C |
| ATOM | 4211 | CG2 | VAL | C | 15 | −5.879 | 50.245 | −11.098 | 1.00 | 34.33 | C |
| ATOM | 4215 | C | VAL | C | 15 | −8.212 | 48.545 | −8.570 | 1.00 | 33.18 | C |
| ATOM | 4216 | O | VAL | C | 15 | −9.391 | 48.458 | −8.842 | 1.00 | 32.31 | O |
| ATOM | 4217 | N | LEU | C | 16 | −7.596 | 47.654 | −7.811 | 1.00 | 41.35 | N |
| ATOM | 4219 | CA | LEU | C | 16 | −8.330 | 46.566 | −7.173 | 1.00 | 38.42 | C |
| ATOM | 4221 | CB | LEU | C | 16 | −7.375 | 45.480 | −6.645 | 1.00 | 33.44 | C |
| ATOM | 4224 | CG | LEU | C | 16 | −7.454 | 44.429 | −7.742 | 1.00 | 52.51 | C |
| ATOM | 4226 | CD1 | LEU | C | 16 | −6.126 | 44.080 | −8.346 | 1.00 | 47.16 | C |
| ATOM | 4230 | CD2 | LEU | C | 16 | −8.285 | 43.206 | −7.360 | 1.00 | 43.26 | C |
| ATOM | 4234 | C | LEU | C | 16 | −9.307 | 46.994 | −6.088 | 1.00 | 31.13 | C |
| ATOM | 4235 | O | LEU | C | 16 | −10.335 | 46.376 | −5.935 | 1.00 | 38.31 | O |
| ATOM | 4236 | N | GLU | C | 17 | −9.028 | 48.042 | −5.333 | 1.00 | 32.97 | N |
| ATOM | 4238 | CA | GLU | C | 17 | −10.073 | 48.601 | −4.493 | 1.00 | 36.24 | C |
| ATOM | 4240 | CB | GLU | C | 17 | −9.571 | 49.819 | −3.733 | 1.00 | 35.40 | C |
| ATOM | 4243 | CG | GLU | C | 17 | −8.750 | 49.570 | −2.469 | 1.00 | 44.99 | C |
| ATOM | 4246 | CD | GLU | C | 17 | −9.214 | 48.401 | −1.638 | 1.00 | 51.99 | C |
| ATOM | 4247 | OE1 | GLU | C | 17 | −9.927 | 48.655 | −0.655 | 1.00 | 76.12 | O |
| ATOM | 4248 | OE2 | GLU | C | 17 | −8.843 | 47.244 | −1.945 | 1.00 | 78.21 | O |
| ATOM | 4249 | C | GLU | C | 17 | −11.319 | 49.008 | −5.289 | 1.00 | 39.15 | C |
| ATOM | 4250 | O | GLU | C | 17 | −12.414 | 49.073 | −4.748 | 1.00 | 45.76 | O |
| ATOM | 4251 | N | ALA | C | 18 | −11.161 | 49.333 | −6.564 | 1.00 | 44.56 | N |
| ATOM | 4253 | CA | ALA | C | 18 | −12.232 | 49.975 | −7.322 | 1.00 | 37.88 | C |
| ATOM | 4255 | CB | ALA | C | 18 | −11.689 | 50.914 | −8.396 | 1.00 | 31.51 | C |
| ATOM | 4259 | C | ALA | C | 18 | −13.035 | 48.903 | −8.007 | 1.00 | 40.45 | C |
| ATOM | 4260 | O | ALA | C | 18 | −14.132 | 49.201 | −8.470 | 1.00 | 49.53 | O |
| ATOM | 4261 | N | LEU | C | 19 | −12.463 | 47.708 | −8.163 | 1.00 | 36.07 | N |
| ATOM | 4263 | CA | LEU | C | 19 | −13.213 | 46.612 | −8.745 | 1.00 | 35.57 | C |
| ATOM | 4265 | CB | LEU | C | 19 | −12.365 | 45.859 | −9.762 | 1.00 | 40.04 | C |
| ATOM | 4268 | CG | LEU | C | 19 | −11.477 | 46.705 | −10.681 | 1.00 | 52.79 | C |
| ATOM | 4270 | CD1 | LEU | C | 19 | −10.355 | 45.918 | −11.394 | 1.00 | 61.00 | C |
| ATOM | 4274 | CD2 | LEU | C | 19 | −12.370 | 47.302 | −11.709 | 1.00 | 62.13 | C |
| ATOM | 4278 | C | LEU | C | 19 | −13.853 | 45.663 | −7.706 | 1.00 | 41.77 | C |
| ATOM | 4279 | O | LEU | C | 19 | −14.950 | 45.154 | −7.983 | 1.00 | 40.45 | O |
| ATOM | 4280 | N | LYS | C | 20 | −13.218 | 45.403 | −6.546 | 1.00 | 41.08 | N |
| ATOM | 4282 | CA | LYS | C | 20 | −13.538 | 44.206 | −5.719 | 1.00 | 34.96 | C |
| ATOM | 4284 | CB | LYS | C | 20 | −12.573 | 43.930 | −4.562 | 1.00 | 33.62 | C |
| ATOM | 4287 | CG | LYS | C | 20 | −12.432 | 45.065 | −3.552 | 1.00 | 39.30 | C |
| ATOM | 4290 | CD | LYS | C | 20 | −12.048 | 44.689 | −2.120 | 1.00 | 39.90 | C |
| ATOM | 4293 | CE | LYS | C | 20 | −12.640 | 45.720 | −1.116 | 1.00 | 58.88 | C |
| ATOM | 4296 | NZ | LYS | C | 20 | −11.650 | 46.427 | −0.221 | 1.00 | 68.80 | N |
| ATOM | 4300 | C | LYS | C | 20 | −14.943 | 44.189 | −5.165 | 1.00 | 32.91 | C |
| ATOM | 4301 | O | LYS | C | 20 | −15.574 | 43.151 | −5.131 | 1.00 | 40.86 | O |
| ATOM | 4302 | N | ASP | C | 21 | −15.501 | 45.350 | −4.868 | 1.00 | 45.87 | N |
| ATOM | 4304 | CA | ASP | C | 21 | −16.869 | 45.380 | −4.352 | 1.00 | 52.82 | C |
| ATOM | 4306 | CB | ASP | C | 21 | −17.133 | 46.708 | −3.622 | 1.00 | 59.76 | C |
| ATOM | 4309 | CG | ASP | C | 21 | −16.263 | 46.880 | −2.390 | 1.00 | 60.08 | C |
| ATOM | 4310 | OD1 | ASP | C | 21 | −15.724 | 45.845 | −1.929 | 1.00 | 55.08 | O |
| ATOM | 4311 | OD2 | ASP | C | 21 | −16.050 | 47.994 | −1.857 | 1.00 | 60.61 | O |
| ATOM | 4312 | C | ASP | C | 21 | −17.954 | 45.102 | −5.408 | 1.00 | 48.64 | C |
| ATOM | 4313 | O | ASP | C | 21 | −19.027 | 44.624 | −5.071 | 1.00 | 52.26 | O |
| ATOM | 4314 | N | LEU | C | 22 | −17.667 | 45.355 | −6.680 | 1.00 | 46.84 | N |
| ATOM | 4316 | CA | LEU | C | 22 | −18.656 | 45.234 | −7.752 | 1.00 | 44.38 | C |
| ATOM | 4318 | CB | LEU | C | 22 | −18.393 | 46.308 | −8.803 | 1.00 | 41.42 | C |
| ATOM | 4321 | CG | LEU | C | 22 | −19.419 | 46.502 | −9.917 | 1.00 | 41.28 | C |
| ATOM | 4323 | CD1 | LEU | C | 22 | −20.858 | 46.861 | −9.398 | 1.00 | 43.30 | C |
| ATOM | 4327 | CD2 | LEU | C | 22 | −18.849 | 47.599 | −10.804 | 1.00 | 24.24 | C |
| ATOM | 4331 | C | LEU | C | 22 | −18.554 | 43.916 | −8.487 | 1.00 | 45.50 | C |
| ATOM | 4332 | O | LEU | C | 22 | −19.547 | 43.255 | −8.841 | 1.00 | 38.97 | O |
| ATOM | 4333 | N | AILE | C | 23 | −17.301 | 43.544 | −8.729 | 0.50 | 50.62 | N |
| ATOM | 4334 | N | BILE | C | 23 | −17.322 | 43.554 | −8.804 | 0.50 | 48.93 | N |
| ATOM | 4337 | CA | AILE | C | 23 | −16.944 | 42.236 | −9.278 | 0.50 | 49.89 | C |
| ATOM | 4338 | CA | BILE | C | 23 | −17.151 | 42.313 | −9.532 | 0.50 | 45.39 | C |
| ATOM | 4341 | CB | AILE | C | 23 | −16.202 | 42.415 | −10.623 | 0.50 | 49.23 | C |
| ATOM | 4342 | CB | BILE | C | 23 | −16.797 | 42.542 | −11.000 | 0.50 | 42.09 | C |
| ATOM | 4345 | CG1 | AILE | C | 23 | −16.979 | 43.404 | −11.497 | 0.50 | 50.67 | C |
| ATOM | 4346 | CG1 | BILE | C | 23 | −15.522 | 43.372 | −11.141 | 0.50 | 38.64 | C |
| ATOM | 4351 | CD1 | AILE | C | 23 | −16.255 | 43.805 | −12.767 | 0.50 | 56.13 | C |
| ATOM | 4352 | CD1 | BILE | C | 23 | −14.640 | 42.876 | −12.264 | 0.50 | 48.64 | C |
| ATOM | 4359 | CG2 | AILE | C | 23 | −16.069 | 41.074 | −11.355 | 0.50 | 53.88 | C |
| ATOM | 4360 | CG2 | BILE | C | 23 | −18.009 | 43.170 | −11.693 | 0.50 | 19.89 | C |
| ATOM | 4367 | C | AILE | C | 23 | −16.122 | 41.411 | −8.280 | 0.50 | 42.92 | C |
| ATOM | 4368 | C | BILE | C | 23 | −16.434 | 41.165 | −8.826 | 0.50 | 45.30 | C |
| ATOM | 4369 | O | AILE | C | 23 | −15.342 | 41.941 | −7.502 | 0.50 | 45.65 | O |
| ATOM | 4370 | O | BILE | C | 23 | −15.286 | 41.233 | −8.398 | 0.50 | 40.57 | O |
| ATOM | 4371 | N | AASN | C | 24 | −16.367 | 40.111 | −8.248 | 0.50 | 37.32 | N |
| ATOM | 4372 | N | BASN | C | 24 | −17.279 | 40.172 | −8.585 | 0.50 | 50.34 | N |
| ATOM | 4375 | CA | AASN | C | 24 | −15.620 | 39.230 | −7.370 | 0.50 | 37.31 | C |
| ATOM | 4376 | CA | BASN | C | 24 | −16.932 | 38.840 | −8.136 | 0.50 | 51.87 | C |
| ATOM | 4379 | CB | AASN | C | 24 | −16.571 | 38.302 | −6.616 | 0.50 | 34.71 | C |
| ATOM | 4380 | CB | BASN | C | 24 | −18.041 | 37.895 | −8.643 | 0.50 | 58.30 | C |
| ATOM | 4385 | CG | AASN | C | 24 | −16.242 | 38.234 | −5.155 | 0.50 | 39.01 | C |
| ATOM | 4386 | CG | BASN | C | 24 | −19.018 | 38.581 | −9.613 | 0.50 | 39.15 | C |
| ATOM | 4387 | OD1 | AASN | C | 24 | −15.960 | 37.158 | −4.644 | 0.50 | 30.52 | O |
| ATOM | 4388 | OD1 | BASN | C | 24 | −19.009 | 38.290 | −10.792 | 0.50 | 29.24 | O |
| ATOM | 4389 | ND2 | AASN | C | 24 | −16.200 | 39.395 | −4.488 | 0.50 | 29.66 | N |
| ATOM | 4390 | ND2 | BASN | C | 24 | −19.784 | 39.547 | −9.131 | 0.50 | 36.36 | N |
| ATOM | 4395 | C | AASN | C | 24 | −14.629 | 38.395 | −8.170 | 0.50 | 39.17 | C |
| ATOM | 4396 | C | BASN | C | 24 | −15.526 | 38.413 | −8.578 | 0.50 | 51.24 | C |
| ATOM | 4397 | O | AASN | C | 24 | −13.667 | 37.875 | −7.625 | 0.50 | 39.58 | O |
| ATOM | 4398 | O | BASN | C | 24 | −14.556 | 38.693 | −7.877 | 0.50 | 50.83 | O |
| ATOM | 4399 | N | AGLU | C | 25 | −14.851 | 38.257 | −9.469 | 0.50 | 40.52 | N |
| ATOM | 4400 | N | BGLU | C | 25 | −15.424 | 37.761 | −9.736 | 0.50 | 48.68 | N |
| ATOM | 4403 | CA | AGLU | C | 25 | −14.042 | 37.340 | −10.258 | 0.50 | 46.66 | C |
| ATOM | 4404 | CA | BGLU | C | 25 | −14.148 | 37.298 | −10.298 | 0.50 | 49.95 | C |
| ATOM | 4407 | CB | AGLU | C | 25 | −14.640 | 35.936 | −10.150 | 0.50 | 52.16 | C |
| ATOM | 4408 | CB | BGLU | C | 25 | −14.096 | 35.765 | −10.306 | 0.50 | 52.34 | C |
| ATOM | 4413 | CG | AGLU | C | 25 | −15.774 | 35.872 | −9.126 | 0.50 | 65.13 | C |
| ATOM | 4414 | CG | BGLU | C | 25 | −15.462 | 35.088 | −10.368 | 0.50 | 61.59 | C |
| ATOM | 4419 | CD | AGLU | C | 25 | −16.191 | 34.457 | −8.770 | 0.50 | 76.69 | C |
| ATOM | 4420 | CD | BGLU | C | 25 | −15.405 | 33.595 | −10.089 | 0.50 | 65.31 | C |
| ATOM | 4421 | OE1 | AGLU | C | 25 | −16.228 | 33.607 | −9.693 | 0.50 | 85.90 | O |
| ATOM | 4422 | OE1 | BGLU | C | 25 | −15.877 | 32.824 | −10.948 | 0.50 | 81.68 | O |
| ATOM | 4423 | OE2 | AGLU | C | 25 | −16.490 | 34.211 | −7.574 | 0.50 | 76.04 | O |
| ATOM | 4424 | OE2 | BGLU | C | 25 | −14.924 | 33.185 | −9.010 | 0.50 | 73.59 | O |
| ATOM | 4425 | C | AGLU | C | 25 | −14.010 | 37.815 | −11.703 | 0.50 | 45.46 | C |
| ATOM | 4426 | C | BGLU | C | 25 | −14.031 | 37.792 | −11.738 | 0.50 | 47.10 | C |
| ATOM | 4427 | O | AGLU | C | 25 | −15.018 | 38.258 | −12.250 | 0.50 | 52.42 | O |
| ATOM | 4428 | O | BGLU | C | 25 | −15.030 | 38.168 | −12.349 | 0.50 | 53.17 | O |
| ATOM | 4429 | N | ALA | C | 26 | −12.839 | 37.796 | −12.313 | 1.00 | 40.67 | N |
| ATOM | 4431 | CA | ALA | C | 26 | −12.743 | 38.274 | −13.677 | 1.00 | 38.22 | C |
| ATOM | 4433 | CB | ALA | C | 26 | −12.840 | 39.742 | −13.717 | 1.00 | 31.94 | C |
| ATOM | 4437 | C | ALA | C | 26 | −11.481 | 37.813 | −14.370 | 1.00 | 42.23 | C |
| ATOM | 4438 | O | ALA | C | 26 | −10.529 | 37.321 | −13.763 | 1.00 | 46.77 | O |
| ATOM | 4439 | N | CYS | C | 27 | −11.490 | 37.923 | −15.681 | 1.00 | 38.96 | N |
| ATOM | 4441 | CA | CYS | C | 27 | −10.358 | 37.425 | −16.390 | 1.00 | 35.47 | C |
| ATOM | 4443 | CB | CYS | C | 27 | −10.822 | 36.549 | −17.555 | 1.00 | 30.03 | C |
| ATOM | 4446 | SG | CYS | C | 27 | −9.656 | 36.612 | −18.910 | 1.00 | 62.73 | S |
| ATOM | 4447 | C | CYS | C | 27 | −9.439 | 38.609 | −16.738 | 1.00 | 37.77 | C |
| ATOM | 4448 | O | CYS | C | 27 | −9.861 | 39.639 | −17.266 | 1.00 | 38.15 | O |
| ATOM | 4449 | N | TRP | C | 28 | −8.173 | 38.482 | −16.362 | 1.00 | 37.42 | N |
| ATOM | 4451 | CA | TRP | C | 28 | −7.190 | 39.491 | −16.705 | 1.00 | 39.27 | C |
| ATOM | 4453 | CB | TRP | C | 28 | −6.147 | 39.604 | −15.610 | 1.00 | 38.40 | C |
| ATOM | 4456 | CG | TRP | C | 28 | −6.662 | 40.175 | −14.372 | 1.00 | 34.08 | C |
| ATOM | 4457 | CD1 | TRP | C | 28 | −7.532 | 39.605 | −13.496 | 1.00 | 46.31 | C |
| ATOM | 4459 | NE1 | TRP | C | 28 | −7.736 | 40.439 | −12.425 | 1.00 | 33.60 | N |
| ATOM | 4461 | CE2 | TRP | C | 28 | −6.950 | 41.552 | −12.585 | 1.00 | 44.43 | C |
| ATOM | 4462 | CD2 | TRP | C | 28 | −6.303 | 41.433 | −13.826 | 1.00 | 34.26 | C |
| ATOM | 4463 | CE3 | TRP | C | 28 | −5.428 | 42.442 | −14.224 | 1.00 | 44.08 | C |
| ATOM | 4465 | CZ3 | TRP | C | 28 | −5.288 | 43.551 | −13.434 | 1.00 | 40.03 | C |
| ATOM | 4467 | CH2 | TRP | C | 28 | −5.945 | 43.648 | −12.201 | 1.00 | 41.52 | C |
| ATOM | 4469 | CZ2 | TRP | C | 28 | −6.778 | 42.664 | −11.757 | 1.00 | 39.41 | C |
| ATOM | 4471 | C | TRP | C | 28 | −6.489 | 39.035 | −17.957 | 1.00 | 38.85 | C |
| ATOM | 4472 | O | TRP | C | 28 | −5.759 | 38.042 | −17.930 | 1.00 | 43.97 | O |
| ATOM | 4473 | N | ASP | C | 29 | −6.722 | 39.761 | −19.041 | 1.00 | 38.53 | N |
| ATOM | 4475 | CA | ASP | C | 29 | −6.165 | 39.396 | −20.340 | 1.00 | 42.48 | C |
| ATOM | 4477 | CB | ASP | C | 29 | −7.032 | 39.912 | −21.485 | 1.00 | 43.57 | C |
| ATOM | 4480 | CG | ASP | C | 29 | −8.350 | 39.207 | −21.549 | 1.00 | 58.48 | C |
| ATOM | 4481 | OD1 | ASP | C | 29 | −8.322 | 38.021 | −21.966 | 1.00 | 56.45 | O |
| ATOM | 4482 | OD2 | ASP | C | 29 | −9.415 | 39.745 | −21.148 | 1.00 | 57.76 | O |
| ATOM | 4483 | C | ASP | C | 29 | −4.828 | 40.063 | −20.475 | 1.00 | 39.93 | C |
| ATOM | 4484 | O | ASP | C | 29 | −4.773 | 41.287 | −20.454 | 1.00 | 39.76 | O |
| ATOM | 4485 | N | ILE | C | 30 | −3.776 | 39.269 | −20.655 | 1.00 | 37.26 | N |
| ATOM | 4487 | CA | ILE | C | 30 | −2.429 | 39.799 | −20.706 | 1.00 | 34.33 | C |
| ATOM | 4489 | CB | ILE | C | 30 | −1.584 | 39.039 | −19.702 | 1.00 | 34.85 | C |
| ATOM | 4491 | CG1 | ILE | C | 30 | −2.227 | 39.017 | −18.315 | 1.00 | 37.83 | C |
| ATOM | 4494 | CD1 | ILE | C | 30 | −2.203 | 40.393 | −17.612 | 1.00 | 59.65 | C |
| ATOM | 4498 | CG2 | ILE | C | 30 | −0.193 | 39.643 | −19.639 | 1.00 | 41.20 | C |
| ATOM | 4502 | C | ILE | C | 30 | −1.833 | 39.667 | −22.114 | 1.00 | 41.35 | C |
| ATOM | 4503 | O | ILE | C | 30 | −1.736 | 38.588 | −22.711 | 1.00 | 40.55 | O |
| ATOM | 4504 | N | SER | C | 31 | −1.451 | 40.790 | −22.694 | 1.00 | 47.50 | N |
| ATOM | 4506 | CA | SER | C | 31 | −0.863 | 40.739 | −24.035 | 1.00 | 45.93 | C |
| ATOM | 4508 | CB | SER | C | 31 | −1.887 | 41.184 | −25.054 | 1.00 | 37.45 | C |
| ATOM | 4511 | OG | SER | C | 31 | −2.158 | 42.539 | −24.812 | 1.00 | 48.06 | O |
| ATOM | 4513 | C | SER | C | 31 | 0.339 | 41.681 | −24.106 | 1.00 | 43.99 | C |
| ATOM | 4514 | O | SER | C | 31 | 0.814 | 42.170 | −23.080 | 1.00 | 45.45 | O |
| ATOM | 4515 | N | SER | C | 32 | 0.867 | 41.887 | −25.305 | 1.00 | 43.21 | N |
| ATOM | 4517 | CA | SER | C | 32 | 2.199 | 42.482 | −25.428 | 1.00 | 48.38 | C |
| ATOM | 4519 | CB | SER | C | 32 | 2.830 | 42.206 | −26.808 | 1.00 | 54.18 | C |
| ATOM | 4522 | OG | SER | C | 32 | 1.852 | 41.793 | −27.766 | 1.00 | 63.24 | O |
| ATOM | 4524 | C | SER | C | 32 | 1.962 | 43.960 | −25.231 | 1.00 | 42.93 | C |
| ATOM | 4525 | O | SER | C | 32 | 2.826 | 44.698 | −24.776 | 1.00 | 49.01 | O |
| ATOM | 4526 | N | SER | C | 33 | 0.729 | 44.343 | −25.544 | 1.00 | 44.58 | N |
| ATOM | 4528 | CA | SER | C | 33 | 0.292 | 45.721 | −25.514 | 1.00 | 42.61 | C |
| ATOM | 4530 | CB | SER | C | 33 | −0.927 | 45.894 | −26.416 | 1.00 | 35.80 | C |
| ATOM | 4533 | OG | SER | C | 33 | −2.021 | 45.125 | −25.989 | 1.00 | 57.93 | O |
| ATOM | 4535 | C | SER | C | 33 | −0.016 | 46.178 | −24.100 | 1.00 | 44.43 | C |
| ATOM | 4536 | O | SER | C | 33 | 0.153 | 47.345 | −23.750 | 1.00 | 57.55 | O |
| ATOM | 4537 | N | GLY | C | 34 | −0.473 | 45.248 | −23.280 | 1.00 | 44.58 | N |
| ATOM | 4539 | CA | GLY | C | 34 | −0.518 | 45.486 | −21.860 | 1.00 | 45.56 | C |
| ATOM | 4542 | C | GLY | C | 34 | −1.636 | 44.648 | −21.269 | 1.00 | 45.39 | C |
| ATOM | 4543 | O | GLY | C | 34 | −1.812 | 43.480 | −21.639 | 1.00 | 43.68 | O |
| ATOM | 4544 | N | VAL | C | 35 | −2.363 | 45.246 | −20.328 | 1.00 | 34.61 | N |
| ATOM | 4546 | CA | VAL | C | 35 | −3.287 | 44.487 | −19.526 | 1.00 | 37.74 | C |
| ATOM | 4548 | CB | VAL | C | 35 | −2.964 | 44.546 | −18.042 | 1.00 | 40.87 | C |
| ATOM | 4550 | CG1 | VAL | C | 35 | −4.079 | 43.832 | −17.374 | 1.00 | 34.77 | C |
| ATOM | 4554 | CG2 | VAL | C | 35 | −1.621 | 43.881 | −17.705 | 1.00 | 34.66 | C |
| ATOM | 4558 | C | VAL | C | 35 | −4.693 | 45.035 | −19.698 | 1.00 | 34.70 | C |
| ATOM | 4559 | O | VAL | C | 35 | −4.931 | 46.218 | −19.575 | 1.00 | 43.26 | O |
| ATOM | 4560 | N | ASN | C | 36 | −5.636 | 44.180 | −20.018 | 1.00 | 35.30 | N |
| ATOM | 4562 | CA | ASN | C | 36 | −7.022 | 44.597 | −20.156 | 1.00 | 39.14 | C |
| ATOM | 4564 | CB | ASN | C | 36 | −7.452 | 44.602 | −21.639 | 1.00 | 35.75 | C |
| ATOM | 4567 | CG | ASN | C | 36 | −6.534 | 45.433 | −22.490 | 1.00 | 49.40 | C |
| ATOM | 4568 | OD1 | ASN | C | 36 | −6.859 | 46.567 | −22.886 | 1.00 | 64.13 | O |
| ATOM | 4569 | ND2 | ASN | C | 36 | −5.307 | 44.935 | −22.653 | 1.00 | 60.79 | N |
| ATOM | 4572 | C | ASN | C | 36 | −7.907 | 43.631 | −19.362 | 1.00 | 41.59 | C |
| ATOM | 4573 | O | ASN | C | 36 | −7.518 | 42.488 | −19.085 | 1.00 | 43.05 | O |
| ATOM | 4574 | N | LEU | C | 37 | −9.115 | 44.078 | −19.038 | 1.00 | 39.82 | N |
| ATOM | 4576 | CA | LEU | C | 37 | −10.041 | 43.272 | −18.264 | 1.00 | 33.50 | C |
| ATOM | 4578 | CB | LEU | C | 37 | −9.670 | 43.358 | −16.795 | 1.00 | 32.71 | C |
| ATOM | 4581 | CG | LEU | C | 37 | −10.723 | 42.811 | −15.838 | 1.00 | 26.85 | C |
| ATOM | 4583 | CD1 | LEU | C | 37 | −10.050 | 42.152 | −14.641 | 1.00 | 44.85 | C |
| ATOM | 4587 | CD2 | LEU | C | 37 | −11.636 | 43.878 | −15.311 | 1.00 | 38.17 | C |
| ATOM | 4591 | C | LEU | C | 37 | −11.412 | 43.866 | −18.470 | 1.00 | 33.37 | C |
| ATOM | 4592 | O | LEU | C | 37 | −11.592 | 45.075 | −18.340 | 1.00 | 34.97 | O |
| ATOM | 4593 | N | GLN | C | 38 | −12.368 | 43.011 | −18.802 | 1.00 | 36.72 | N |
| ATOM | 4595 | CA | GLN | C | 38 | −13.771 | 43.393 | −18.981 | 1.00 | 35.97 | C |
| ATOM | 4597 | CB | GLN | C | 38 | −14.130 | 43.411 | −20.474 | 1.00 | 29.40 | C |
| ATOM | 4600 | CG | GLN | C | 38 | −15.518 | 43.931 | −20.825 | 1.00 | 38.46 | C |
| ATOM | 4603 | CD | GLN | C | 38 | −15.938 | 43.545 | −22.242 | 1.00 | 46.78 | C |
| ATOM | 4604 | OE1 | GLN | C | 38 | −16.627 | 42.539 | −22.459 | 1.00 | 43.64 | O |
| ATOM | 4605 | NE2 | GLN | C | 38 | −15.512 | 44.343 | −23.206 | 1.00 | 33.15 | N |
| ATOM | 4608 | C | GLN | C | 38 | −14.676 | 42.371 | −18.275 | 1.00 | 43.47 | C |
| ATOM | 4609 | O | GLN | C | 38 | −14.616 | 41.157 | −18.520 | 1.00 | 37.47 | O |
| ATOM | 4610 | N | SER | C | 39 | −15.579 | 42.890 | −17.454 | 1.00 | 43.23 | N |
| ATOM | 4612 | CA | SER | C | 39 | −16.524 | 42.043 | −16.764 | 1.00 | 45.45 | C |
| ATOM | 4614 | CB | SER | C | 39 | −15.939 | 41.706 | −15.401 | 1.00 | 46.26 | C |
| ATOM | 4617 | OG | SER | C | 39 | −16.673 | 40.648 | −14.856 | 1.00 | 50.98 | O |
| ATOM | 4619 | C | SER | C | 39 | −17.812 | 42.816 | −16.538 | 1.00 | 44.34 | C |
| ATOM | 4620 | O | SER | C | 39 | −17.735 | 43.995 | −16.163 | 1.00 | 40.70 | O |
| ATOM | 4621 | N | MET | C | 40 | −18.961 | 42.146 | −16.677 | 1.00 | 40.59 | N |
| ATOM | 4623 | CA | MET | C | 40 | −20.216 | 42.672 | −16.130 | 1.00 | 46.69 | C |
| ATOM | 4625 | CB | MET | C | 40 | −21.491 | 42.177 | −16.854 | 1.00 | 44.51 | C |
| ATOM | 4628 | CG | MET | C | 40 | −21.662 | 42.678 | −18.294 | 1.00 | 49.29 | C |
| ATOM | 4631 | SD | MET | C | 40 | −22.925 | 41.780 | −19.221 | 1.00 | 43.47 | S |
| ATOM | 4632 | CE | MET | C | 40 | −22.614 | 40.143 | −18.791 | 1.00 | 61.47 | C |
| ATOM | 4636 | C | MET | C | 40 | −20.277 | 42.359 | −14.644 | 1.00 | 41.36 | C |
| ATOM | 4637 | O | MET | C | 40 | −19.519 | 41.549 | −14.179 | 1.00 | 40.31 | O |
| ATOM | 4638 | N | ASP | C | 41 | −21.150 | 43.034 | −13.903 | 1.00 | 38.83 | N |
| ATOM | 4640 | CA | ASP | C | 41 | −21.429 | 42.618 | −12.558 | 1.00 | 39.51 | C |
| ATOM | 4642 | CB | ASP | C | 41 | −21.901 | 43.802 | −11.734 | 1.00 | 35.14 | C |
| ATOM | 4645 | CG | ASP | C | 41 | −23.304 | 44.162 | −12.025 | 1.00 | 42.24 | C |
| ATOM | 4646 | OD1 | ASP | C | 41 | −24.151 | 43.856 | −11.169 | 1.00 | 48.23 | O |
| ATOM | 4647 | OD2 | ASP | C | 41 | −23.656 | 44.740 | −13.070 | 1.00 | 53.67 | O |
| ATOM | 4648 | C | ASP | C | 41 | −22.452 | 41.492 | −12.622 | 1.00 | 45.72 | C |
| ATOM | 4649 | O | ASP | C | 41 | −22.905 | 41.130 | −13.718 | 1.00 | 51.45 | O |
| ATOM | 4650 | N | SER | C | 42 | −22.747 | 40.884 | −11.473 | 1.00 | 45.16 | N |
| ATOM | 4652 | CA | SER | C | 42 | −23.713 | 39.789 | −11.407 | 1.00 | 44.24 | C |
| ATOM | 4654 | CB | SER | C | 42 | −24.057 | 39.489 | −9.964 | 1.00 | 47.71 | C |
| ATOM | 4657 | OG | SER | C | 42 | −23.176 | 38.482 | −9.512 | 1.00 | 55.07 | O |
| ATOM | 4659 | C | SER | C | 42 | −24.991 | 40.096 | −12.157 | 1.00 | 41.32 | C |
| ATOM | 4660 | O | SER | C | 42 | −25.540 | 39.240 | −12.804 | 1.00 | 45.74 | O |
| ATOM | 4661 | N | SER | C | 43 | −25.448 | 41.333 | −12.098 | 1.00 | 41.85 | N |
| ATOM | 4663 | CA | SER | C | 43 | −26.796 | 41.675 | −12.513 | 1.00 | 40.94 | C |
| ATOM | 4665 | CB | SER | C | 43 | −27.337 | 42.785 | −11.596 | 1.00 | 48.51 | C |
| ATOM | 4668 | OG | SER | C | 43 | −26.846 | 44.094 | −11.885 | 1.00 | 39.06 | O |
| ATOM | 4670 | C | SER | C | 43 | −26.840 | 42.122 | −13.957 | 1.00 | 46.34 | C |
| ATOM | 4671 | O | SER | C | 43 | −27.898 | 42.490 | −14.456 | 1.00 | 48.79 | O |
| ATOM | 4672 | N | HIS | C | 44 | −25.703 | 42.054 | −14.639 | 1.00 | 48.82 | N |
| ATOM | 4674 | CA | HIS | C | 44 | −25.613 | 42.372 | −16.068 | 1.00 | 49.44 | C |
| ATOM | 4676 | CB | HIS | C | 44 | −26.317 | 41.324 | −16.923 | 1.00 | 49.44 | C |
| ATOM | 4679 | CG | HIS | C | 44 | −25.653 | 39.979 | −16.909 | 1.00 | 56.33 | C |
| ATOM | 4680 | ND1 | HIS | C | 44 | −26.053 | 38.942 | −17.732 | 1.00 | 49.10 | N |
| ATOM | 4682 | CE1 | HIS | C | 44 | −25.290 | 37.887 | −17.500 | 1.00 | 57.04 | C |
| ATOM | 4684 | NE2 | HIS | C | 44 | −24.390 | 38.207 | −16.582 | 1.00 | 53.63 | N |
| ATOM | 4686 | CD2 | HIS | C | 44 | −24.604 | 39.507 | −16.187 | 1.00 | 56.86 | C |
| ATOM | 4688 | C | HIS | C | 44 | −26.090 | 43.787 | −16.393 | 1.00 | 45.94 | C |
| ATOM | 4689 | O | HIS | C | 44 | −26.417 | 44.135 | −17.526 | 1.00 | 50.99 | O |
| ATOM | 4690 | N | VAL | C | 45 | −26.000 | 44.650 | −15.398 | 1.00 | 37.96 | N |
| ATOM | 4692 | CA | VAL | C | 45 | −26.483 | 46.007 | −15.548 | 1.00 | 33.94 | C |
| ATOM | 4694 | CB | VAL | C | 45 | −27.260 | 46.314 | −14.259 | 1.00 | 32.80 | C |
| ATOM | 4696 | CG1 | VAL | C | 45 | −27.536 | 47.790 | −14.142 | 1.00 | 38.97 | C |
| ATOM | 4700 | CG2 | VAL | C | 45 | −28.564 | 45.534 | −14.244 | 1.00 | 36.49 | C |
| ATOM | 4704 | C | VAL | C | 45 | −25.332 | 47.015 | −15.724 | 1.00 | 33.88 | C |
| ATOM | 4705 | O | VAL | C | 45 | −25.517 | 48.174 | −16.083 | 1.00 | 28.69 | O |
| ATOM | 4706 | N | SER | C | 46 | −24.145 | 46.607 | −15.302 | 1.00 | 38.60 | N |
| ATOM | 4708 | CA | SER | C | 46 | −22.968 | 47.448 | −15.369 | 1.00 | 41.04 | C |
| ATOM | 4710 | CB | SER | C | 46 | −22.561 | 48.002 | −13.999 | 1.00 | 45.25 | C |
| ATOM | 4713 | OG | SER | C | 46 | −22.262 | 46.969 | −13.084 | 1.00 | 48.62 | O |
| ATOM | 4715 | C | SER | C | 46 | −21.841 | 46.620 | −15.945 | 1.00 | 37.68 | C |
| ATOM | 4716 | O | SER | C | 46 | −21.810 | 45.394 | −15.875 | 1.00 | 32.64 | O |
| ATOM | 4717 | N | LEU | C | 47 | −20.900 | 47.302 | −16.563 | 1.00 | 37.04 | N |
| ATOM | 4719 | CA | LEU | C | 47 | −19.739 | 46.575 | −17.029 | 1.00 | 36.83 | C |
| ATOM | 4721 | CB | LEU | C | 47 | −19.903 | 46.116 | −18.476 | 1.00 | 36.91 | C |
| ATOM | 4724 | CG | LEU | C | 47 | −18.675 | 45.817 | −19.323 | 1.00 | 43.15 | C |
| ATOM | 4726 | CD1 | LEU | C | 47 | −18.977 | 44.947 | −20.540 | 1.00 | 60.67 | C |
| ATOM | 4730 | CD2 | LEU | C | 47 | −18.056 | 47.115 | −19.742 | 1.00 | 41.79 | C |
| ATOM | 4734 | C | LEU | C | 47 | −18.520 | 47.458 | −16.830 | 1.00 | 39.77 | C |
| ATOM | 4735 | O | LEU | C | 47 | −18.547 | 48.699 | −16.873 | 1.00 | 36.96 | O |
| ATOM | 4736 | N | VAL | C | 48 | −17.455 | 46.760 | −16.499 | 1.00 | 33.70 | N |
| ATOM | 4738 | CA | VAL | C | 48 | −16.229 | 47.420 | −16.236 | 1.00 | 35.62 | C |
| ATOM | 4740 | CB | VAL | C | 48 | −15.800 | 47.002 | −14.865 | 1.00 | 36.97 | C |
| ATOM | 4742 | CG1 | VAL | C | 48 | −14.594 | 46.128 | −14.945 | 1.00 | 54.05 | C |
| ATOM | 4746 | CG2 | VAL | C | 48 | −15.541 | 48.186 | −14.031 | 1.00 | 42.62 | C |
| ATOM | 4750 | C | VAL | C | 48 | −15.201 | 47.113 | −17.331 | 1.00 | 34.59 | C |
| ATOM | 4751 | O | VAL | C | 48 | −15.233 | 46.111 | −18.029 | 1.00 | 30.47 | O |
| ATOM | 4752 | N | GLN | C | 49 | −14.235 | 47.998 | −17.474 | 1.00 | 38.46 | N |
| ATOM | 4754 | CA | GLN | C | 49 | −13.264 | 47.870 | −18.537 | 1.00 | 28.35 | C |
| ATOM | 4756 | CB | GLN | C | 49 | −13.856 | 48.497 | −19.786 | 1.00 | 30.87 | C |
| ATOM | 4759 | CG | GLN | C | 49 | −13.299 | 47.955 | −21.076 | 1.00 | 50.72 | C |
| ATOM | 4762 | CD | GLN | C | 49 | −12.081 | 48.739 | −21.473 | 1.00 | 74.27 | C |
| ATOM | 4763 | OE1 | GLN | C | 49 | −12.209 | 49.838 | −22.027 | 1.00 | 69.81 | O |
| ATOM | 4764 | NE2 | GLN | C | 49 | −10.895 | 48.211 | −21.137 | 1.00 | 91.85 | N |
| ATOM | 4767 | C | GLN | C | 49 | −11.998 | 48.577 | −18.068 | 1.00 | 27.63 | C |
| ATOM | 4768 | O | GLN | C | 49 | −12.039 | 49.719 | −17.645 | 1.00 | 29.49 | O |
| ATOM | 4769 | N | LEU | C | 50 | −10.885 | 47.858 | −18.042 | 1.00 | 37.79 | N |
| ATOM | 4771 | CA | LEU | C | 50 | −9.641 | 48.351 | −17.443 | 1.00 | 37.21 | C |
| ATOM | 4773 | CB | LEU | C | 50 | −9.197 | 47.464 | −16.274 | 1.00 | 44.84 | C |
| ATOM | 4776 | CG | LEU | C | 50 | −7.803 | 47.695 | −15.684 | 1.00 | 25.24 | C |
| ATOM | 4778 | CD1 | LEU | C | 50 | −7.837 | 48.924 | −14.797 | 1.00 | 28.69 | C |
| ATOM | 4782 | CD2 | LEU | C | 50 | −7.375 | 46.554 | −14.834 | 1.00 | 30.05 | C |
| ATOM | 4786 | C | LEU | C | 50 | −8.570 | 48.233 | −18.487 | 1.00 | 35.85 | C |
| ATOM | 4787 | O | LEU | C | 50 | −8.357 | 47.152 | −19.046 | 1.00 | 41.29 | O |
| ATOM | 4788 | N | THR | C | 51 | −7.877 | 49.333 | −18.728 | 1.00 | 33.50 | N |
| ATOM | 4790 | CA | THR | C | 51 | −6.746 | 49.289 | −19.638 | 1.00 | 35.96 | C |
| ATOM | 4792 | CB | THR | C | 51 | −7.017 | 50.215 | −20.823 | 1.00 | 40.98 | C |
| ATOM | 4794 | OG1 | THR | C | 51 | −8.220 | 49.817 | −21.486 | 1.00 | 47.54 | O |
| ATOM | 4796 | CG2 | THR | C | 51 | −5.942 | 50.085 | −21.872 | 1.00 | 48.68 | C |
| ATOM | 4800 | C | THR | C | 51 | −5.511 | 49.759 | −18.883 | 1.00 | 37.62 | C |
| ATOM | 4801 | O | THR | C | 51 | −5.471 | 50.902 | −18.418 | 1.00 | 39.18 | O |
| ATOM | 4802 | N | LEU | C | 52 | −4.531 | 48.874 | −18.712 | 1.00 | 35.61 | N |
| ATOM | 4804 | CA | LEU | C | 52 | −3.180 | 49.303 | −18.333 | 1.00 | 39.99 | C |
| ATOM | 4806 | CB | LEU | C | 52 | −2.741 | 48.619 | −17.042 | 1.00 | 38.26 | C |
| ATOM | 4809 | CG | LEU | C | 52 | −3.707 | 48.831 | −15.883 | 1.00 | 40.87 | C |
| ATOM | 4811 | CD1 | LEU | C | 52 | −3.382 | 47.962 | −14.678 | 1.00 | 51.27 | C |
| ATOM | 4815 | CD2 | LEU | C | 52 | −3.595 | 50.256 | −15.491 | 1.00 | 42.43 | C |
| ATOM | 4819 | C | LEU | C | 52 | −2.168 | 48.998 | −19.439 | 1.00 | 40.89 | C |
| ATOM | 4820 | O | LEU | C | 52 | −1.853 | 47.828 | −19.717 | 1.00 | 40.41 | O |
| ATOM | 4821 | N | ARG | C | 53 | −1.687 | 50.063 | −20.071 | 1.00 | 33.17 | N |
| ATOM | 4823 | CA | ARG | C | 53 | −0.721 | 49.971 | −21.159 | 1.00 | 35.72 | C |
| ATOM | 4825 | CB | ARG | C | 53 | −0.531 | 51.363 | −21.760 | 1.00 | 27.81 | C |
| ATOM | 4828 | CG | ARG | C | 53 | −1.507 | 51.664 | −22.865 | 1.00 | 19.26 | C |
| ATOM | 4831 | CD | ARG | C | 53 | −1.407 | 53.073 | −23.480 | 1.00 | 34.48 | C |
| ATOM | 4834 | NE | ARG | C | 53 | −2.014 | 54.103 | −22.636 | 1.00 | 55.98 | N |
| ATOM | 4836 | CZ | ARG | C | 53 | −1.326 | 54.973 | −21.923 | 1.00 | 44.55 | C |
| ATOM | 4837 | NH1 | ARG | C | 53 | 0.003 | 54.990 | −21.981 | 1.00 | 74.78 | N |
| ATOM | 4840 | NH2 | ARG | C | 53 | −1.974 | 55.814 | −21.139 | 1.00 | 65.66 | N |
| ATOM | 4843 | C | ARG | C | 53 | 0.642 | 49.490 | −20.671 | 1.00 | 35.28 | C |
| ATOM | 4844 | O | ARG | C | 53 | 1.060 | 49.858 | −19.589 | 1.00 | 43.24 | O |
| ATOM | 4845 | N | SER | C | 54 | 1.367 | 48.736 | −21.486 | 1.00 | 39.91 | N |
| ATOM | 4847 | CA | SER | C | 54 | 2.650 | 48.119 | −21.086 | 1.00 | 41.23 | C |
| ATOM | 4849 | CB | SER | C | 54 | 3.217 | 47.198 | −22.183 | 1.00 | 42.39 | C |
| ATOM | 4852 | OG | SER | C | 54 | 3.527 | 47.912 | −23.388 | 1.00 | 49.11 | O |
| ATOM | 4854 | C | SER | C | 54 | 3.710 | 49.133 | −20.766 | 1.00 | 36.55 | C |
| ATOM | 4855 | O | SER | C | 54 | 4.546 | 48.897 | −19.902 | 1.00 | 48.79 | O |
| ATOM | 4856 | N | GLU | C | 55 | 3.712 | 50.213 | −21.537 | 1.00 | 39.29 | N |
| ATOM | 4858 | CA | GLU | C | 55 | 4.621 | 51.340 | −21.342 | 1.00 | 38.70 | C |
| ATOM | 4860 | CB | GLU | C | 55 | 4.135 | 52.507 | −22.188 | 1.00 | 42.51 | C |
| ATOM | 4863 | CG | GLU | C | 55 | 3.961 | 52.109 | −23.634 | 1.00 | 55.24 | C |
| ATOM | 4866 | CD | GLU | C | 55 | 2.516 | 52.004 | −24.014 | 1.00 | 70.75 | C |
| ATOM | 4867 | OE1 | GLU | C | 55 | 1.908 | 53.113 | −24.047 | 1.00 | 71.37 | O |
| ATOM | 4868 | OE2 | GLU | C | 55 | 2.061 | 50.843 | −24.251 | 1.00 | 54.70 | O |
| ATOM | 4869 | C | GLU | C | 55 | 4.632 | 51.841 | −19.923 | 1.00 | 34.96 | C |
| ATOM | 4870 | O | GLU | C | 55 | 5.541 | 52.543 | −19.525 | 1.00 | 50.70 | O |
| ATOM | 4871 | N | GLY | C | 56 | 3.580 | 51.541 | −19.176 | 1.00 | 37.93 | N |
| ATOM | 4873 | CA | GLY | C | 56 | 3.247 | 52.292 | −17.985 | 1.00 | 34.60 | C |
| ATOM | 4876 | C | GLY | C | 56 | 3.903 | 51.593 | −16.825 | 1.00 | 32.04 | C |
| ATOM | 4877 | O | GLY | C | 56 | 4.026 | 52.173 | −15.726 | 1.00 | 30.75 | O |
| ATOM | 4878 | N | PHE | C | 57 | 4.209 | 50.319 | −17.096 | 1.00 | 30.90 | N |
| ATOM | 4880 | CA | PHE | C | 57 | 5.090 | 49.484 | −16.284 | 1.00 | 30.77 | C |
| ATOM | 4882 | CB | PHE | C | 57 | 4.852 | 48.032 | −16.617 | 1.00 | 26.49 | C |
| ATOM | 4885 | CG | PHE | C | 57 | 3.514 | 47.542 | −16.191 | 1.00 | 28.33 | C |
| ATOM | 4886 | CD1 | PHE | C | 57 | 2.417 | 47.627 | −17.026 | 1.00 | 22.56 | C |
| ATOM | 4888 | CE1 | PHE | C | 57 | 1.174 | 47.156 | −16.622 | 1.00 | 30.03 | C |
| ATOM | 4890 | CZ | PHE | C | 57 | 0.992 | 46.651 | −15.349 | 1.00 | 19.99 | C |
| ATOM | 4892 | CE2 | PHE | C | 57 | 2.063 | 46.648 | −14.496 | 1.00 | 45.11 | C |
| ATOM | 4894 | CD2 | PHE | C | 57 | 3.325 | 47.086 | −14.911 | 1.00 | 24.39 | C |
| ATOM | 4896 | C | PHE | C | 57 | 6.520 | 49.764 | −16.633 | 1.00 | 34.28 | C |
| ATOM | 4897 | O | PHE | C | 57 | 6.820 | 50.221 | −17.723 | 1.00 | 45.87 | O |
| ATOM | 4898 | N | ASP | C | 58 | 7.411 | 49.472 | −15.703 | 1.00 | 45.28 | N |
| ATOM | 4900 | CA | ASP | C | 58 | 8.763 | 49.993 | −15.769 | 1.00 | 44.11 | C |
| ATOM | 4902 | CB | ASP | C | 58 | 9.310 | 50.259 | −14.369 | 1.00 | 50.82 | C |
| ATOM | 4905 | CG | ASP | C | 58 | 10.817 | 50.123 | −14.298 | 1.00 | 65.21 | C |
| ATOM | 4906 | OD1 | ASP | C | 58 | 11.531 | 50.975 | −14.880 | 1.00 | 66.71 | O |
| ATOM | 4907 | OD2 | ASP | C | 58 | 11.368 | 49.203 | −13.654 | 1.00 | 93.47 | O |
| ATOM | 4908 | C | ASP | C | 58 | 9.528 | 48.939 | −16.520 | 1.00 | 41.00 | C |
| ATOM | 4909 | O | ASP | C | 58 | 10.429 | 49.250 | −17.277 | 1.00 | 54.97 | O |
| ATOM | 4910 | N | THR | C | 59 | 8.973 | 47.735 | −16.483 | 1.00 | 43.71 | N |
| ATOM | 4912 | CA | THR | C | 59 | 9.428 | 46.558 | −17.209 | 1.00 | 31.69 | C |
| ATOM | 4914 | CB | THR | C | 59 | 10.118 | 45.626 | −16.199 | 1.00 | 45.66 | C |
| ATOM | 4916 | OG1 | THR | C | 59 | 11.522 | 45.870 | −16.240 | 1.00 | 37.81 | O |
| ATOM | 4918 | CG2 | THR | C | 59 | 9.836 | 44.073 | −16.464 | 1.00 | 42.54 | C |
| ATOM | 4922 | C | THR | C | 59 | 8.134 | 45.868 | −17.570 | 1.00 | 38.64 | C |
| ATOM | 4923 | O | THR | C | 59 | 7.166 | 45.860 | −16.787 | 1.00 | 41.41 | O |
| ATOM | 4924 | N | TYR | C | 60 | 8.166 | 45.152 | −18.679 | 1.00 | 34.72 | N |
| ATOM | 4926 | CA | TYR | C | 60 | 6.978 | 44.450 | −19.125 | 1.00 | 34.32 | C |
| ATOM | 4928 | CB | TYR | C | 60 | 6.002 | 45.416 | −19.835 | 1.00 | 34.40 | C |
| ATOM | 4931 | CG | TYR | C | 60 | 4.647 | 44.796 | −20.045 | 1.00 | 28.60 | C |
| ATOM | 4932 | CD1 | TYR | C | 60 | 3.752 | 44.741 | −19.011 | 1.00 | 27.36 | C |
| ATOM | 4934 | CE1 | TYR | C | 60 | 2.533 | 44.124 | −19.151 | 1.00 | 35.98 | C |
| ATOM | 4936 | CZ | TYR | C | 60 | 2.214 | 43.522 | −20.323 | 1.00 | 40.37 | C |
| ATOM | 4937 | OH | TYR | C | 60 | 0.988 | 42.934 | −20.404 | 1.00 | 36.91 | O |
| ATOM | 4939 | CE2 | TYR | C | 60 | 3.082 | 43.563 | −21.389 | 1.00 | 42.80 | C |
| ATOM | 4941 | CD2 | TYR | C | 60 | 4.303 | 44.175 | −21.235 | 1.00 | 39.24 | C |
| ATOM | 4943 | C | TYR | C | 60 | 7.407 | 43.339 | −20.069 | 1.00 | 33.96 | C |
| ATOM | 4944 | O | TYR | C | 60 | 8.339 | 43.517 | −20.829 | 1.00 | 38.74 | O |
| ATOM | 4945 | N | ARG | C | 61 | 6.748 | 42.188 | −20.016 | 1.00 | 37.24 | N |
| ATOM | 4947 | CA | ARG | C | 61 | 7.255 | 40.983 | −20.658 | 1.00 | 36.45 | C |
| ATOM | 4949 | CB | ARG | C | 61 | 8.353 | 40.271 | −19.846 | 1.00 | 40.53 | C |
| ATOM | 4952 | CG | ARG | C | 61 | 8.884 | 38.936 | −20.457 | 1.00 | 47.67 | C |
| ATOM | 4955 | CD | ARG | C | 61 | 10.367 | 38.651 | −20.154 | 1.00 | 68.01 | C |
| ATOM | 4958 | NE | ARG | C | 61 | 10.660 | 37.359 | −19.513 | 1.00 | 69.02 | N |
| ATOM | 4960 | CZ | ARG | C | 61 | 10.558 | 36.192 | −20.138 | 1.00 | 68.30 | C |
| ATOM | 4961 | NH1 | ARG | C | 61 | 10.130 | 36.155 | −21.388 | 1.00 | 75.25 | N |
| ATOM | 4964 | NH2 | ARG | C | 61 | 10.845 | 35.053 | −19.530 | 1.00 | 58.50 | N |
| ATOM | 4967 | C | ARG | C | 61 | 6.074 | 40.071 | −20.795 | 1.00 | 39.07 | C |
| ATOM | 4968 | O | ARG | C | 61 | 5.403 | 39.715 | −19.824 | 1.00 | 42.79 | O |
| ATOM | 4969 | N | CYS | C | 62 | 5.807 | 39.676 | −22.023 | 1.00 | 43.96 | N |
| ATOM | 4971 | CA | CYS | C | 62 | 4.688 | 38.780 | −22.214 | 1.00 | 43.20 | C |
| ATOM | 4973 | CB | CYS | C | 62 | 3.431 | 39.598 | −22.417 | 1.00 | 43.12 | C |
| ATOM | 4976 | SG | CYS | C | 62 | 2.029 | 38.532 | −22.687 | 1.00 | 48.72 | S |
| ATOM | 4977 | C | CYS | C | 62 | 4.965 | 37.880 | −23.401 | 1.00 | 45.68 | C |
| ATOM | 4978 | O | CYS | C | 62 | 4.549 | 38.144 | −24.529 | 1.00 | 52.65 | O |
| ATOM | 4979 | N | ASP | C | 63 | 5.742 | 36.840 | −23.138 | 1.00 | 52.21 | N |
| ATOM | 4981 | CA | ASP | C | 63 | 6.039 | 35.805 | −24.132 | 1.00 | 51.15 | C |
| ATOM | 4983 | CB | ASP | C | 63 | 6.775 | 34.642 | −23.470 | 1.00 | 46.94 | C |
| ATOM | 4986 | CG | ASP | C | 63 | 8.105 | 35.058 | −22.943 | 1.00 | 43.99 | C |
| ATOM | 4987 | OD1 | ASP | C | 63 | 8.756 | 35.880 | −23.615 | 1.00 | 52.66 | O |
| ATOM | 4988 | OD2 | ASP | C | 63 | 8.559 | 34.661 | −21.861 | 1.00 | 60.10 | O |
| ATOM | 4989 | C | ASP | C | 63 | 4.818 | 35.292 | −24.874 | 1.00 | 51.42 | C |
| ATOM | 4990 | O | ASP | C | 63 | 4.884 | 35.159 | −26.082 | 1.00 | 51.95 | O |
| ATOM | 4991 | N | ARG | C | 64 | 3.715 | 35.062 | −24.160 | 1.00 | 52.56 | N |
| ATOM | 4993 | CA | ARG | C | 64 | 2.550 | 34.394 | −24.722 | 1.00 | 56.93 | C |
| ATOM | 4995 | CB | ARG | C | 64 | 2.555 | 32.879 | −24.401 | 1.00 | 58.02 | C |
| ATOM | 4998 | CG | ARG | C | 64 | 3.190 | 32.016 | −25.501 | 1.00 | 81.22 | C |
| ATOM | 5001 | CD | ARG | C | 64 | 2.232 | 31.476 | −26.587 | 1.00 | 100.08 | C |
| ATOM | 5004 | NE | ARG | C | 64 | 2.044 | 30.034 | −26.424 | 1.00 | 119.88 | N |
| ATOM | 5006 | CZ | ARG | C | 64 | 2.956 | 29.112 | −26.722 | 1.00 | 123.35 | C |
| ATOM | 5007 | NH1 | ARG | C | 64 | 4.113 | 29.473 | −27.259 | 1.00 | 114.20 | N |
| ATOM | 5010 | NH2 | ARG | C | 64 | 2.711 | 27.825 | −26.499 | 1.00 | 126.01 | N |
| ATOM | 5013 | C | ARG | C | 64 | 1.347 | 35.068 | −24.104 | 1.00 | 45.82 | C |
| ATOM | 5014 | O | ARG | C | 64 | 1.390 | 35.366 | −22.925 | 1.00 | 48.58 | O |
| ATOM | 5015 | N | ASN | C | 65 | 0.273 | 35.256 | −24.865 | 1.00 | 40.70 | N |
| ATOM | 5017 | CA | ASN | C | 65 | −0.944 | 35.852 | −24.311 | 1.00 | 40.90 | C |
| ATOM | 5019 | CB | ASN | C | 65 | −2.008 | 36.051 | −25.383 | 1.00 | 39.29 | C |
| ATOM | 5022 | CG | ASN | C | 65 | −1.688 | 37.170 | −26.346 | 1.00 | 35.72 | C |
| ATOM | 5023 | OD1 | ASN | C | 65 | −2.505 | 37.437 | −27.211 | 1.00 | 51.97 | O |
| ATOM | 5024 | ND2 | ASN | C | 65 | −0.539 | 37.843 | −26.199 | 1.00 | 42.00 | N |
| ATOM | 5027 | C | ASN | C | 65 | −1.598 | 35.010 | −23.237 | 1.00 | 43.21 | C |
| ATOM | 5028 | O | ASN | C | 65 | −1.562 | 33.797 | −23.272 | 1.00 | 53.65 | O |
| ATOM | 5029 | N | LEU | C | 66 | −2.310 | 35.646 | −22.330 | 1.00 | 45.63 | N |
| ATOM | 5031 | CA | LEU | C | 66 | −2.657 | 34.967 | −21.100 | 1.00 | 44.27 | C |
| ATOM | 5033 | CB | LEU | C | 66 | −1.678 | 35.391 | −20.027 | 1.00 | 46.04 | C |
| ATOM | 5036 | CG | LEU | C | 66 | −0.694 | 34.343 | −19.565 | 1.00 | 48.61 | C |
| ATOM | 5038 | CD1 | LEU | C | 66 | 0.000 | 34.972 | −18.388 | 1.00 | 62.78 | C |
| ATOM | 5042 | CD2 | LEU | C | 66 | −1.466 | 33.108 | −19.147 | 1.00 | 75.29 | C |
| ATOM | 5046 | C | LEU | C | 66 | −4.043 | 35.378 | −20.658 | 1.00 | 47.56 | C |
| ATOM | 5047 | O | LEU | C | 66 | −4.365 | 36.569 | −20.676 | 1.00 | 40.97 | O |
| ATOM | 5048 | N | ALA | C | 67 | −4.853 | 34.417 | −20.220 | 1.00 | 47.74 | N |
| ATOM | 5050 | CA | ALA | C | 67 | −6.117 | 34.824 | −19.629 | 1.00 | 49.65 | C |
| ATOM | 5052 | CB | ALA | C | 67 | −7.291 | 34.447 | −20.512 | 1.00 | 40.00 | C |
| ATOM | 5056 | C | ALA | C | 67 | −6.302 | 34.307 | −18.214 | 1.00 | 50.37 | C |
| ATOM | 5057 | O | ALA | C | 67 | −6.849 | 33.225 | −18.010 | 1.00 | 60.50 | O |
| ATOM | 5058 | N | MET | C | 68 | −5.909 | 35.138 | −17.252 | 1.00 | 45.87 | N |
| ATOM | 5060 | CA | MET | C | 68 | −5.831 | 34.737 | −15.862 | 1.00 | 37.70 | C |
| ATOM | 5062 | CB | MET | C | 68 | −4.845 | 35.667 | −15.178 | 1.00 | 36.34 | C |
| ATOM | 5065 | CG | MET | C | 68 | −3.488 | 35.069 | −15.048 | 1.00 | 43.44 | C |
| ATOM | 5068 | SD | MET | C | 68 | −2.127 | 36.173 | −14.960 | 1.00 | 49.17 | S |
| ATOM | 5069 | CE | MET | C | 68 | −2.956 | 37.534 | −14.255 | 1.00 | 53.68 | C |
| ATOM | 5073 | C | MET | C | 68 | −7.208 | 34.885 | −15.244 | 1.00 | 37.77 | C |
| ATOM | 5074 | O | MET | C | 68 | −7.745 | 35.976 | −15.183 | 1.00 | 46.33 | O |
| ATOM | 5075 | N | GLY | C | 69 | −7.813 | 33.796 | −14.798 | 1.00 | 46.48 | N |
| ATOM | 5077 | CA | GLY | C | 69 | −9.022 | 33.922 | −14.002 | 1.00 | 43.33 | C |
| ATOM | 5080 | C | GLY | C | 69 | −8.710 | 34.173 | −12.538 | 1.00 | 45.77 | C |
| ATOM | 5081 | O | GLY | C | 69 | −8.000 | 33.390 | −11.896 | 1.00 | 49.04 | O |
| ATOM | 5082 | N | VAL | C | 70 | −9.224 | 35.272 | −12.009 | 1.00 | 37.08 | N |
| ATOM | 5084 | CA | VAL | C | 70 | −8.766 | 35.757 | −10.733 | 1.00 | 35.63 | C |
| ATOM | 5086 | CB | VAL | C | 70 | −7.994 | 37.049 | −10.919 | 1.00 | 38.58 | C |
| ATOM | 5088 | CG1 | VAL | C | 70 | −7.971 | 37.869 | −9.608 | 1.00 | 41.79 | C |
| ATOM | 5092 | CG2 | VAL | C | 70 | −6.603 | 36.740 | −11.416 | 1.00 | 48.98 | C |
| ATOM | 5096 | C | VAL | C | 70 | −9.963 | 36.107 | −9.903 | 1.00 | 36.94 | C |
| ATOM | 5097 | O | VAL | C | 70 | −10.788 | 36.910 | −10.350 | 1.00 | 40.55 | O |
| ATOM | 5098 | N | ASN | C | 71 | −9.976 | 35.636 | −8.657 | 1.00 | 41.11 | N |
| ATOM | 5100 | CA | ASN | C | 71 | −10.816 | 36.216 | −7.596 | 1.00 | 35.15 | C |
| ATOM | 5102 | CB | ASN | C | 71 | −11.019 | 35.203 | −6.475 | 1.00 | 36.59 | C |
| ATOM | 5105 | CG | ASN | C | 71 | −12.012 | 35.649 | −5.393 | 1.00 | 39.24 | C |
| ATOM | 5106 | OD1 | ASN | C | 71 | −11.886 | 36.716 | −4.786 | 1.00 | 40.28 | O |
| ATOM | 5107 | ND2 | ASN | C | 71 | −12.943 | 34.767 | −5.067 | 1.00 | 42.69 | N |
| ATOM | 5110 | C | ASN | C | 71 | −10.234 | 37.530 | −7.066 | 1.00 | 38.21 | C |
| ATOM | 5111 | O | ASN | C | 71 | −9.196 | 37.571 | −6.392 | 1.00 | 38.53 | O |
| ATOM | 5112 | N | LEU | C | 72 | −10.934 | 38.606 | −7.422 | 1.00 | 36.72 | N |
| ATOM | 5114 | CA | LEU | C | 72 | −10.544 | 39.972 | −7.126 | 1.00 | 28.67 | C |
| ATOM | 5116 | CB | LEU | C | 72 | −11.468 | 40.953 | −7.834 | 1.00 | 30.41 | C |
| ATOM | 5119 | CG | LEU | C | 72 | −11.600 | 40.800 | −9.349 | 1.00 | 30.76 | C |
| ATOM | 5121 | CD1 | LEU | C | 72 | −12.263 | 42.032 | −9.923 | 1.00 | 40.79 | C |
| ATOM | 5125 | CD2 | LEU | C | 72 | −10.267 | 40.536 | −10.015 | 1.00 | 37.06 | C |
| ATOM | 5129 | C | LEU | C | 72 | −10.628 | 40.280 | −5.655 | 1.00 | 30.03 | C |
| ATOM | 5130 | O | LEU | C | 72 | −9.895 | 41.127 | −5.163 | 1.00 | 22.84 | O |
| ATOM | 5131 | N | THR | C | 73 | −11.452 | 39.562 | −4.901 | 1.00 | 40.07 | N |
| ATOM | 5133 | CA | THR | C | 73 | −11.227 | 39.580 | −3.447 | 1.00 | 41.69 | C |
| ATOM | 5135 | CB | THR | C | 73 | −12.373 | 38.927 | −2.725 | 1.00 | 38.21 | C |
| ATOM | 5137 | OG1 | THR | C | 73 | −13.614 | 39.523 | −3.129 | 1.00 | 45.72 | O |
| ATOM | 5139 | CG2 | THR | C | 73 | −12.253 | 39.236 | −1.250 | 1.00 | 38.74 | C |
| ATOM | 5143 | C | THR | C | 73 | −9.895 | 38.981 | −2.948 | 1.00 | 41.36 | C |
| ATOM | 5144 | O | THR | C | 73 | −9.134 | 39.589 | −2.191 | 1.00 | 32.54 | O |
| ATOM | 5145 | N | SER | C | 74 | −9.573 | 37.788 | −3.414 | 1.00 | 40.44 | N |
| ATOM | 5147 | CA | SER | C | 74 | −8.234 | 37.307 | −3.176 | 1.00 | 43.19 | C |
| ATOM | 5149 | CB | SER | C | 74 | −8.052 | 35.972 | −3.868 | 1.00 | 40.47 | C |
| ATOM | 5152 | OG | SER | C | 74 | −9.031 | 35.086 | −3.356 | 1.00 | 38.50 | O |
| ATOM | 5154 | C | SER | C | 74 | −7.155 | 38.323 | −3.558 | 1.00 | 43.94 | C |
| ATOM | 5155 | O | SER | C | 74 | −6.214 | 38.573 | −2.817 | 1.00 | 48.84 | O |
| ATOM | 5156 | N | MET | C | 75 | −7.236 | 38.916 | −4.730 | 1.00 | 47.39 | N |
| ATOM | 5158 | CA | MET | C | 75 | −6.065 | 39.640 | −5.192 | 1.00 | 41.94 | C |
| ATOM | 5160 | CB | MET | C | 75 | −6.245 | 39.954 | −6.668 | 1.00 | 42.29 | C |
| ATOM | 5163 | CG | MET | C | 75 | −5.166 | 40.790 | −7.270 | 1.00 | 30.92 | C |
| ATOM | 5166 | SD | MET | C | 75 | −5.288 | 40.643 | −9.042 | 1.00 | 35.22 | S |
| ATOM | 5167 | CE | MET | C | 75 | −4.088 | 41.801 | −9.506 | 1.00 | 31.93 | C |
| ATOM | 5171 | C | MET | C | 75 | −5.983 | 40.898 | −4.344 | 1.00 | 41.31 | C |
| ATOM | 5172 | O | MET | C | 75 | −4.891 | 41.313 | −3.963 | 1.00 | 50.27 | O |
| ATOM | 5173 | N | SER | C | 76 | −7.142 | 41.422 | −3.956 | 1.00 | 33.23 | N |
| ATOM | 5175 | CA | SER | C | 76 | −7.214 | 42.508 | −2.991 | 1.00 | 38.78 | C |
| ATOM | 5177 | CB | SER | C | 76 | −8.659 | 42.901 | −2.680 | 1.00 | 45.15 | C |
| ATOM | 5180 | OG | SER | C | 76 | −8.628 | 44.094 | −1.911 | 1.00 | 55.27 | O |
| ATOM | 5182 | C | SER | C | 76 | −6.545 | 42.225 | −1.655 | 1.00 | 32.62 | C |
| ATOM | 5183 | O | SER | C | 76 | −5.805 | 43.048 | −1.129 | 1.00 | 41.82 | O |
| ATOM | 5184 | N | LYS | C | 77 | −6.852 | 41.093 | −1.048 | 1.00 | 31.34 | N |
| ATOM | 5186 | CA | LYS | C | 77 | −6.346 | 40.845 | 0.295 | 1.00 | 30.34 | C |
| ATOM | 5188 | CB | LYS | C | 77 | −6.871 | 39.548 | 0.902 | 1.00 | 34.50 | C |
| ATOM | 5191 | CG | LYS | C | 77 | −8.374 | 39.465 | 1.067 | 1.00 | 38.88 | C |
| ATOM | 5194 | CD | LYS | C | 77 | −8.813 | 39.424 | 2.540 | 1.00 | 74.96 | C |
| ATOM | 5197 | CE | LYS | C | 77 | −9.243 | 38.029 | 2.978 | 1.00 | 72.32 | C |
| ATOM | 5200 | NZ | LYS | C | 77 | −9.341 | 37.114 | 1.803 | 1.00 | 74.04 | N |
| ATOM | 5204 | C | LYS | C | 77 | −4.843 | 40.753 | 0.234 | 1.00 | 33.49 | C |
| ATOM | 5205 | O | LYS | C | 77 | −4.173 | 41.198 | 1.153 | 1.00 | 42.64 | O |
| ATOM | 5206 | N | ILE | C | 78 | −4.312 | 40.208 | −0.852 | 1.00 | 33.45 | N |
| ATOM | 5208 | CA | ILE | C | 78 | −2.877 | 40.232 | −1.080 | 1.00 | 36.63 | C |
| ATOM | 5210 | CB | ILE | C | 78 | −2.536 | 39.380 | −2.310 | 1.00 | 39.29 | C |
| ATOM | 5212 | CG1 | ILE | C | 78 | −2.340 | 37.915 | −1.901 | 1.00 | 35.63 | C |
| ATOM | 5215 | CD1 | ILE | C | 78 | −2.864 | 36.851 | −2.869 | 1.00 | 36.92 | C |
| ATOM | 5219 | CG2 | ILE | C | 78 | −1.213 | 39.796 | −2.884 | 1.00 | 56.29 | C |
| ATOM | 5223 | C | ILE | C | 78 | −2.285 | 41.633 | −1.196 | 1.00 | 39.37 | C |
| ATOM | 5224 | O | ILE | C | 78 | −1.184 | 41.945 | −0.679 | 1.00 | 39.90 | O |
| ATOM | 5225 | N | LEU | C | 79 | −2.998 | 42.491 | −1.911 | 1.00 | 41.10 | N |
| ATOM | 5227 | CA | LEU | C | 79 | −2.405 | 43.770 | −2.279 | 1.00 | 33.83 | C |
| ATOM | 5229 | CB | LEU | C | 79 | −3.148 | 44.329 | −3.482 | 1.00 | 26.13 | C |
| ATOM | 5232 | CG | LEU | C | 79 | −2.671 | 43.716 | −4.799 | 1.00 | 37.15 | C |
| ATOM | 5234 | CD1 | LEU | C | 79 | −3.561 | 44.074 | −5.964 | 1.00 | 44.30 | C |
| ATOM | 5238 | CD2 | LEU | C | 79 | −1.252 | 44.162 | −5.115 | 1.00 | 50.09 | C |
| ATOM | 5242 | C | LEU | C | 79 | −2.487 | 44.709 | −1.088 | 1.00 | 38.47 | C |
| ATOM | 5243 | O | LEU | C | 79 | −1.767 | 45.726 | −1.000 | 1.00 | 33.49 | O |
| ATOM | 5244 | N | LYS | C | 80 | −3.412 | 44.382 | −0.189 | 1.00 | 38.44 | N |
| ATOM | 5246 | CA | LYS | C | 80 | −3.449 | 45.079 | 1.086 | 1.00 | 44.41 | C |
| ATOM | 5248 | CB | LYS | C | 80 | −4.680 | 44.724 | 1.941 | 1.00 | 51.34 | C |
| ATOM | 5251 | CG | LYS | C | 80 | −6.021 | 45.213 | 1.391 | 1.00 | 58.00 | C |
| ATOM | 5254 | CD | LYS | C | 80 | −6.692 | 46.242 | 2.281 | 1.00 | 75.80 | C |
| ATOM | 5257 | CE | LYS | C | 80 | −7.505 | 47.256 | 1.481 | 1.00 | 82.68 | C |
| ATOM | 5260 | NZ | LYS | C | 80 | −8.709 | 47.664 | 2.252 | 1.00 | 76.08 | N |
| ATOM | 5264 | C | LYS | C | 80 | −2.158 | 44.792 | 1.844 | 1.00 | 36.41 | C |
| ATOM | 5265 | O | LYS | C | 80 | −1.769 | 45.588 | 2.663 | 1.00 | 46.16 | O |
| ATOM | 5266 | N | CYS | C | 81 | −1.453 | 43.699 | 1.589 | 1.00 | 39.63 | N |
| ATOM | 5268 | CA | CYS | C | 81 | −0.147 | 43.506 | 2.252 | 1.00 | 45.47 | C |
| ATOM | 5270 | CB | CYS | C | 81 | 0.225 | 42.036 | 2.349 | 1.00 | 41.99 | C |
| ATOM | 5273 | SG | CYS | C | 81 | −1.164 | 40.952 | 2.730 | 1.00 | 40.50 | S |
| ATOM | 5274 | C | CYS | C | 81 | 1.058 | 44.234 | 1.636 | 1.00 | 47.20 | C |
| ATOM | 5275 | O | CYS | C | 81 | 2.207 | 43.976 | 2.022 | 1.00 | 54.54 | O |
| ATOM | 5276 | N | ALA | C | 82 | 0.787 | 45.121 | 0.683 | 1.00 | 41.57 | N |
| ATOM | 5278 | CA | ALA | C | 82 | 1.811 | 45.887 | −0.018 | 1.00 | 32.54 | C |
| ATOM | 5280 | CB | ALA | C | 82 | 1.602 | 45.922 | −1.575 | 1.00 | 28.59 | C |
| ATOM | 5284 | C | ALA | C | 82 | 1.549 | 47.251 | 0.533 | 1.00 | 28.76 | C |
| ATOM | 5285 | O | ALA | C | 82 | 0.447 | 47.512 | 0.980 | 1.00 | 41.36 | O |
| ATOM | 5286 | N | GLY | C | 83 | 2.562 | 48.102 | 0.499 | 1.00 | 25.55 | N |
| ATOM | 5288 | CA | GLY | C | 83 | 2.413 | 49.460 | 0.946 | 1.00 | 32.60 | C |
| ATOM | 5291 | C | GLY | C | 83 | 2.418 | 50.311 | −0.303 | 1.00 | 46.37 | C |
| ATOM | 5292 | O | GLY | C | 83 | 2.631 | 49.775 | −1.438 | 1.00 | 44.88 | O |
| ATOM | 5293 | N | ASN | C | 84 | 2.158 | 51.609 | −0.112 | 1.00 | 44.06 | N |
| ATOM | 5295 | CA | ASN | C | 84 | 1.777 | 52.409 | −1.263 | 1.00 | 51.05 | C |
| ATOM | 5297 | CB | ASN | C | 84 | 1.003 | 53.650 | −0.888 | 1.00 | 56.31 | C |
| ATOM | 5300 | CG | ASN | C | 84 | −0.415 | 53.307 | −0.517 | 1.00 | 62.45 | C |
| ATOM | 5301 | OD1 | ASN | C | 84 | −0.645 | 52.934 | 0.628 | 1.00 | 52.83 | O |
| ATOM | 5302 | ND2 | ASN | C | 84 | −1.343 | 53.325 | −1.496 | 1.00 | 52.17 | N |
| ATOM | 5305 | C | ASN | C | 84 | 2.958 | 52.741 | −2.112 | 1.00 | 49.39 | C |
| ATOM | 5306 | O | ASN | C | 84 | 2.824 | 52.861 | −3.340 | 1.00 | 48.88 | O |
| ATOM | 5307 | N | GLU | C | 85 | 4.102 | 52.778 | −1.432 | 1.00 | 49.05 | N |
| ATOM | 5309 | CA | GLU | C | 85 | 5.376 | 53.129 | −2.033 | 1.00 | 47.57 | C |
| ATOM | 5311 | CB | GLU | C | 85 | 6.265 | 53.811 | −0.989 | 1.00 | 55.25 | C |
| ATOM | 5314 | CG | GLU | C | 85 | 6.571 | 55.285 | −1.248 | 1.00 | 61.95 | C |
| ATOM | 5317 | CD | GLU | C | 85 | 5.322 | 56.149 | −1.254 | 1.00 | 77.59 | C |
| ATOM | 5318 | OE1 | GLU | C | 85 | 4.892 | 56.607 | −0.167 | 1.00 | 96.77 | O |
| ATOM | 5319 | OE2 | GLU | C | 85 | 4.764 | 56.372 | −2.352 | 1.00 | 67.76 | O |
| ATOM | 5320 | C | GLU | C | 85 | 6.049 | 51.874 | −2.568 | 1.00 | 40.25 | C |
| ATOM | 5321 | O | GLU | C | 85 | 7.069 | 51.949 | −3.250 | 1.00 | 36.98 | O |
| ATOM | 5322 | N | ASP | C | 86 | 5.470 | 50.716 | −2.276 | 1.00 | 36.53 | N |
| ATOM | 5324 | CA | ASP | C | 86 | 6.079 | 49.465 | −2.717 | 1.00 | 40.91 | C |
| ATOM | 5326 | CB | ASP | C | 86 | 5.413 | 48.223 | −2.084 | 1.00 | 33.43 | C |
| ATOM | 5329 | CG | ASP | C | 86 | 5.681 | 48.110 | −0.607 | 1.00 | 35.93 | C |
| ATOM | 5330 | OD1 | ASP | C | 86 | 6.143 | 49.119 | −0.016 | 1.00 | 62.52 | O |
| ATOM | 5331 | OD2 | ASP | C | 86 | 5.401 | 47.083 | 0.063 | 1.00 | 32.15 | O |
| ATOM | 5332 | C | ASP | C | 86 | 6.109 | 49.375 | −4.257 | 1.00 | 44.24 | C |
| ATOM | 5333 | O | ASP | C | 86 | 5.163 | 49.725 | −4.980 | 1.00 | 39.14 | O |
| ATOM | 5334 | N | ILE | C | 87 | 7.236 | 48.888 | −4.756 | 1.00 | 46.82 | N |
| ATOM | 5336 | CA | ILE | C | 87 | 7.327 | 48.470 | −6.139 | 1.00 | 42.09 | C |
| ATOM | 5338 | CB | ILE | C | 87 | 8.786 | 48.331 | −6.580 | 1.00 | 37.01 | C |
| ATOM | 5340 | CG1 | ILE | C | 87 | 9.480 | 49.695 | −6.560 | 1.00 | 48.39 | C |
| ATOM | 5343 | CD1 | ILE | C | 87 | 10.989 | 49.628 | −6.175 | 1.00 | 54.24 | C |
| ATOM | 5347 | CG2 | ILE | C | 87 | 8.887 | 47.759 | −7.988 | 1.00 | 37.43 | C |
| ATOM | 5351 | C | ILE | C | 87 | 6.668 | 47.121 | −6.224 | 1.00 | 45.22 | C |
| ATOM | 5352 | O | ILE | C | 87 | 7.084 | 46.171 | −5.555 | 1.00 | 41.21 | O |
| ATOM | 5353 | N | ILE | C | 88 | 5.675 | 47.029 | −7.101 | 1.00 | 40.96 | N |
| ATOM | 5355 | CA | ILE | C | 88 | 4.980 | 45.772 | −7.257 | 1.00 | 37.35 | C |
| ATOM | 5357 | CB | ILE | C | 88 | 3.489 | 46.046 | −7.149 | 1.00 | 34.97 | C |
| ATOM | 5359 | CG1 | ILE | C | 88 | 3.187 | 46.513 | −5.723 | 1.00 | 31.57 | C |
| ATOM | 5362 | CD1 | ILE | C | 88 | 1.736 | 46.622 | −5.412 | 1.00 | 42.63 | C |
| ATOM | 5366 | CG2 | ILE | C | 88 | 2.693 | 44.811 | −7.546 | 1.00 | 31.59 | C |
| ATOM | 5370 | C | ILE | C | 88 | 5.302 | 45.065 | −8.553 | 1.00 | 35.59 | C |
| ATOM | 5371 | O | ILE | C | 88 | 5.295 | 45.694 | −9.603 | 1.00 | 37.91 | O |
| ATOM | 5372 | N | THR | C | 89 | 5.500 | 43.749 | −8.479 | 1.00 | 40.12 | N |
| ATOM | 5374 | CA | THR | C | 89 | 5.855 | 42.961 | −9.651 | 1.00 | 36.84 | C |
| ATOM | 5376 | CB | THR | C | 89 | 7.281 | 42.388 | −9.549 | 1.00 | 42.67 | C |
| ATOM | 5378 | OG1 | THR | C | 89 | 8.293 | 43.413 | −9.455 | 1.00 | 30.59 | O |
| ATOM | 5380 | CG2 | THR | C | 89 | 7.613 | 41.685 | −10.839 | 1.00 | 35.14 | C |
| ATOM | 5384 | C | THR | C | 89 | 4.882 | 41.808 | −9.799 | 1.00 | 44.26 | C |
| ATOM | 5385 | O | THR | C | 89 | 4.783 | 40.895 | −8.991 | 1.00 | 41.98 | O |
| ATOM | 5386 | N | LEU | C | 90 | 4.126 | 41.864 | −10.873 | 1.00 | 49.64 | N |
| ATOM | 5388 | CA | LEU | C | 90 | 3.289 | 40.750 | −11.222 | 1.00 | 48.62 | C |
| ATOM | 5390 | CB | LEU | C | 90 | 2.119 | 41.287 | −12.042 | 1.00 | 47.87 | C |
| ATOM | 5393 | CG | LEU | C | 90 | 1.275 | 42.261 | −11.226 | 1.00 | 43.65 | C |
| ATOM | 5395 | CD1 | LEU | C | 90 | 0.040 | 42.698 | −12.024 | 1.00 | 43.86 | C |
| ATOM | 5399 | CD2 | LEU | C | 90 | 0.850 | 41.578 | −9.915 | 1.00 | 45.18 | C |
| ATOM | 5403 | C | LEU | C | 90 | 4.148 | 39.762 | −12.005 | 1.00 | 45.22 | C |
| ATOM | 5404 | O | LEU | C | 90 | 5.004 | 40.174 | −12.779 | 1.00 | 54.33 | O |
| ATOM | 5405 | N | ARG | C | 91 | 3.926 | 38.466 | −11.826 | 1.00 | 41.49 | N |
| ATOM | 5407 | CA | ARG | C | 91 | 4.636 | 37.464 | −12.619 | 1.00 | 44.60 | C |
| ATOM | 5409 | CB | ARG | C | 91 | 6.045 | 37.251 | −12.067 | 1.00 | 46.48 | C |
| ATOM | 5412 | CG | ARG | C | 91 | 6.785 | 36.093 | −12.689 | 1.00 | 37.26 | C |
| ATOM | 5415 | CD | ARG | C | 91 | 8.259 | 36.135 | −12.374 | 1.00 | 37.39 | C |
| ATOM | 5418 | NE | ARG | C | 91 | 8.402 | 36.135 | −10.926 | 1.00 | 70.06 | N |
| ATOM | 5420 | CZ | ARG | C | 91 | 9.130 | 36.990 | −10.224 | 1.00 | 73.40 | C |
| ATOM | 5421 | NH1 | ARG | C | 91 | 9.874 | 37.929 | −10.827 | 1.00 | 47.98 | N |
| ATOM | 5424 | NH2 | ARG | C | 91 | 9.108 | 36.860 | −8.900 | 1.00 | 65.26 | N |
| ATOM | 5427 | C | ARG | C | 91 | 3.937 | 36.122 | −12.702 | 1.00 | 32.11 | C |
| ATOM | 5428 | O | ARG | C | 91 | 3.444 | 35.645 | −11.711 | 1.00 | 33.41 | O |
| ATOM | 5429 | N | ALA | C | 92 | 3.861 | 35.571 | −13.908 | 1.00 | 36.59 | N |
| ATOM | 5431 | CA | ALA | C | 92 | 3.075 | 34.367 | −14.196 | 1.00 | 46.45 | C |
| ATOM | 5433 | CB | ALA | C | 92 | 1.673 | 34.701 | −14.648 | 1.00 | 48.35 | C |
| ATOM | 5437 | C | ALA | C | 92 | 3.749 | 33.584 | −15.291 | 1.00 | 52.37 | C |
| ATOM | 5438 | O | ALA | C | 92 | 4.236 | 34.125 | −16.290 | 1.00 | 51.53 | O |
| ATOM | 5439 | N | GLU | C | 93 | 3.771 | 32.278 | −15.095 | 1.00 | 64.26 | N |
| ATOM | 5441 | CA | GLU | C | 93 | 4.227 | 31.401 | −16.158 | 1.00 | 69.70 | C |
| ATOM | 5443 | CB | GLU | C | 93 | 4.409 | 29.998 | −15.607 | 1.00 | 65.96 | C |
| ATOM | 5446 | CG | GLU | C | 93 | 5.202 | 29.989 | −14.309 | 1.00 | 75.81 | C |
| ATOM | 5449 | CD | GLU | C | 93 | 6.696 | 29.781 | −14.504 | 1.00 | 90.77 | C |
| ATOM | 5450 | OE1 | GLU | C | 93 | 7.076 | 29.095 | −15.476 | 1.00 | 87.39 | O |
| ATOM | 5451 | OE2 | GLU | C | 93 | 7.493 | 30.286 | −13.675 | 1.00 | 94.17 | O |
| ATOM | 5452 | C | GLU | C | 93 | 3.172 | 31.467 | −17.247 | 1.00 | 73.51 | C |
| ATOM | 5453 | O | GLU | C | 93 | 2.214 | 32.225 | −17.115 | 1.00 | 69.39 | O |
| ATOM | 5454 | N | ASP | C | 94 | 3.395 | 30.744 | −18.341 | 1.00 | 83.21 | N |
| ATOM | 5456 | CA | ASP | C | 94 | 2.585 | 30.881 | −19.558 | 1.00 | 86.16 | C |
| ATOM | 5458 | CB | ASP | C | 94 | 3.355 | 30.399 | −20.793 | 1.00 | 85.90 | C |
| ATOM | 5461 | CG | ASP | C | 94 | 4.665 | 31.113 | −20.984 | 1.00 | 78.82 | C |
| ATOM | 5462 | OD1 | ASP | C | 94 | 4.595 | 32.226 | −21.518 | 1.00 | 61.18 | O |
| ATOM | 5463 | OD2 | ASP | C | 94 | 5.780 | 30.664 | −20.634 | 1.00 | 76.74 | O |
| ATOM | 5464 | C | ASP | C | 94 | 1.310 | 30.048 | −19.440 | 1.00 | 89.40 | C |
| ATOM | 5465 | O | ASP | C | 94 | 0.204 | 30.541 | −19.689 | 1.00 | 89.55 | O |
| ATOM | 5466 | N | ASN | C | 95 | 1.473 | 28.770 | −19.108 | 1.00 | 90.75 | N |
| ATOM | 5468 | CA | ASN | C | 95 | 0.346 | 27.844 | −19.124 | 1.00 | 94.04 | C |
| ATOM | 5470 | CB | ASN | C | 95 | 0.657 | 26.540 | −19.895 | 1.00 | 96.68 | C |
| ATOM | 5473 | CG | ASN | C | 95 | −0.600 | 25.882 | −20.489 | 1.00 | 96.95 | C |
| ATOM | 5474 | OD1 | ASN | C | 95 | −0.810 | 25.915 | −21.706 | 1.00 | 92.33 | O |
| ATOM | 5475 | ND2 | ASN | C | 95 | −1.454 | 25.320 | −19.627 | 1.00 | 79.26 | N |
| ATOM | 5478 | C | ASN | C | 95 | 0.121 | 27.494 | −17.689 | 1.00 | 89.19 | C |
| ATOM | 5479 | O | ASN | C | 95 | 0.588 | 26.453 | −17.240 | 1.00 | 92.85 | O |
| ATOM | 5480 | N | ALA | C | 96 | −0.475 | 28.396 | −16.926 | 1.00 | 86.42 | N |
| ATOM | 5482 | CA | ALA | C | 96 | −0.432 | 28.169 | −15.480 | 1.00 | 85.90 | C |
| ATOM | 5484 | CB | ALA | C | 96 | 0.974 | 28.443 | −14.877 | 1.00 | 90.48 | C |
| ATOM | 5488 | C | ALA | C | 96 | −1.499 | 28.822 | −14.637 | 1.00 | 72.56 | C |
| ATOM | 5489 | O | ALA | C | 96 | −2.417 | 29.498 | −15.115 | 1.00 | 55.64 | O |
| ATOM | 5490 | N | ASP | C | 97 | −1.407 | 28.483 | −13.364 | 1.00 | 66.07 | N |
| ATOM | 5492 | CA | ASP | C | 97 | −2.611 | 28.408 | −12.567 | 1.00 | 71.72 | C |
| ATOM | 5494 | CB | ASP | C | 97 | −3.048 | 26.958 | −12.423 | 1.00 | 73.59 | C |
| ATOM | 5497 | CG | ASP | C | 97 | −1.888 | 26.064 | −12.162 | 1.00 | 81.54 | C |
| ATOM | 5498 | OD1 | ASP | C | 97 | −1.980 | 24.882 | −12.550 | 1.00 | 101.69 | O |
| ATOM | 5499 | OD2 | ASP | C | 97 | −0.840 | 26.487 | −11.612 | 1.00 | 96.98 | O |
| ATOM | 5500 | C | ASP | C | 97 | −2.305 | 29.025 | −11.218 | 1.00 | 63.75 | C |
| ATOM | 5501 | O | ASP | C | 97 | −3.120 | 28.956 | −10.289 | 1.00 | 59.12 | O |
| ATOM | 5502 | N | THR | C | 98 | −1.126 | 29.638 | −11.145 | 1.00 | 56.37 | N |
| ATOM | 5504 | CA | THR | C | 98 | −0.874 | 30.629 | −10.136 | 1.00 | 49.63 | C |
| ATOM | 5506 | CB | THR | C | 98 | 0.000 | 30.061 | −9.018 | 1.00 | 44.38 | C |
| ATOM | 5508 | OG1 | THR | C | 98 | 1.308 | 30.659 | −9.073 | 1.00 | 49.34 | O |
| ATOM | 5510 | CG2 | THR | C | 98 | 0.193 | 28.561 | −9.185 | 1.00 | 53.28 | C |
| ATOM | 5514 | C | THR | C | 98 | −0.289 | 31.897 | −10.715 | 1.00 | 47.68 | C |
| ATOM | 5515 | O | THR | C | 98 | 0.369 | 31.906 | −11.739 | 1.00 | 49.27 | O |
| ATOM | 5516 | N | LEU | C | 99 | −0.579 | 32.981 | −10.021 | 1.00 | 45.14 | N |
| ATOM | 5518 | CA | LEU | C | 99 | 0.080 | 34.237 | −10.256 | 1.00 | 49.79 | C |
| ATOM | 5520 | CB | LEU | C | 99 | −0.980 | 35.270 | −10.655 | 1.00 | 52.03 | C |
| ATOM | 5523 | CG | LEU | C | 99 | −0.601 | 36.738 | −10.466 | 1.00 | 39.66 | C |
| ATOM | 5525 | CD1 | LEU | C | 99 | 0.089 | 37.281 | −11.743 | 1.00 | 29.72 | C |
| ATOM | 5529 | CD2 | LEU | C | 99 | −1.826 | 37.561 | −10.132 | 1.00 | 46.25 | C |
| ATOM | 5533 | C | LEU | C | 99 | 0.804 | 34.711 | −8.990 | 1.00 | 51.28 | C |
| ATOM | 5534 | O | LEU | C | 99 | 0.279 | 34.649 | −7.855 | 1.00 | 43.10 | O |
| ATOM | 5535 | N | ALA | C | 100 | 1.988 | 35.258 | −9.246 | 1.00 | 42.66 | N |
| ATOM | 5537 | CA | ALA | C | 100 | 2.953 | 35.557 | −8.211 | 1.00 | 39.99 | C |
| ATOM | 5539 | CB | ALA | C | 100 | 4.334 | 35.021 | −8.585 | 1.00 | 33.17 | C |
| ATOM | 5543 | C | ALA | C | 100 | 2.992 | 37.055 | −8.115 | 1.00 | 38.11 | C |
| ATOM | 5544 | O | ALA | C | 100 | 3.153 | 37.725 | −9.111 | 1.00 | 36.07 | O |
| ATOM | 5545 | N | LEU | C | 101 | 2.848 | 37.575 | −6.904 | 1.00 | 45.99 | N |
| ATOM | 5547 | CA | LEU | C | 101 | 2.978 | 39.009 | −6.653 | 1.00 | 44.30 | C |
| ATOM | 5549 | CB | LEU | C | 101 | 1.760 | 39.546 | −5.907 | 1.00 | 35.31 | C |
| ATOM | 5552 | CG | LEU | C | 101 | 0.533 | 39.548 | −6.791 | 1.00 | 43.13 | C |
| ATOM | 5554 | CD1 | LEU | C | 101 | −0.250 | 38.355 | −6.286 | 1.00 | 41.97 | C |
| ATOM | 5558 | CD2 | LEU | C | 101 | −0.233 | 40.885 | −6.688 | 1.00 | 32.41 | C |
| ATOM | 5562 | C | LEU | C | 101 | 4.183 | 39.205 | −5.766 | 1.00 | 40.66 | C |
| ATOM | 5563 | O | LEU | C | 101 | 4.394 | 38.404 | −4.867 | 1.00 | 50.83 | O |
| ATOM | 5564 | N | VAL | C | 102 | 4.913 | 40.290 | −5.999 | 1.00 | 38.33 | N |
| ATOM | 5566 | CA | VAL | C | 102 | 6.124 | 40.610 | −5.267 | 1.00 | 38.30 | C |
| ATOM | 5568 | CB | VAL | C | 102 | 7.406 | 40.238 | −6.042 | 1.00 | 36.15 | C |
| ATOM | 5570 | CG1 | VAL | C | 102 | 8.611 | 40.556 | −5.182 | 1.00 | 45.23 | C |
| ATOM | 5574 | CG2 | VAL | C | 102 | 7.432 | 38.748 | −6.330 | 1.00 | 32.14 | C |
| ATOM | 5578 | C | VAL | C | 102 | 6.168 | 42.090 | −4.917 | 1.00 | 36.06 | C |
| ATOM | 5579 | O | VAL | C | 102 | 6.089 | 42.973 | −5.770 | 1.00 | 38.40 | O |
| ATOM | 5580 | N | PHE | C | 103 | 6.277 | 42.343 | −3.625 | 1.00 | 34.46 | N |
| ATOM | 5582 | CA | PHE | C | 103 | 6.248 | 43.703 | −3.117 | 1.00 | 37.64 | C |
| ATOM | 5584 | CB | PHE | C | 103 | 5.181 | 43.842 | −2.042 | 1.00 | 29.46 | C |
| ATOM | 5587 | CG | PHE | C | 103 | 3.850 | 43.329 | −2.469 | 1.00 | 34.35 | C |
| ATOM | 5588 | CD1 | PHE | C | 103 | 3.498 | 43.360 | −3.822 | 1.00 | 30.47 | C |
| ATOM | 5590 | CE1 | PHE | C | 103 | 2.241 | 42.938 | −4.211 | 1.00 | 41.68 | C |
| ATOM | 5592 | CZ | PHE | C | 103 | 1.351 | 42.415 | −3.247 | 1.00 | 23.17 | C |
| ATOM | 5594 | CE2 | PHE | C | 103 | 1.720 | 42.350 | −1.892 | 1.00 | 23.31 | C |
| ATOM | 5596 | CD2 | PHE | C | 103 | 2.963 | 42.811 | −1.520 | 1.00 | 24.00 | C |
| ATOM | 5598 | C | PHE | C | 103 | 7.579 | 43.993 | −2.481 | 1.00 | 38.76 | C |
| ATOM | 5599 | O | PHE | C | 103 | 7.966 | 43.277 | −1.551 | 1.00 | 47.01 | O |
| ATOM | 5600 | N | GLU | C | 104 | 8.265 | 45.017 | −2.987 | 1.00 | 36.58 | N |
| ATOM | 5602 | CA | GLU | C | 104 | 9.573 | 45.359 | −2.463 | 1.00 | 45.25 | C |
| ATOM | 5604 | CB | GLU | C | 104 | 10.714 | 44.903 | −3.368 | 1.00 | 41.96 | C |
| ATOM | 5607 | CG | GLU | C | 104 | 10.782 | 45.579 | −4.697 | 1.00 | 56.25 | C |
| ATOM | 5610 | CD | GLU | C | 104 | 11.695 | 44.819 | −5.619 | 1.00 | 70.72 | C |
| ATOM | 5611 | OE1 | GLU | C | 104 | 12.907 | 45.070 | −5.519 | 1.00 | 76.43 | O |
| ATOM | 5612 | OE2 | GLU | C | 104 | 11.210 | 43.976 | −6.401 | 1.00 | 57.93 | O |
| ATOM | 5613 | C | GLU | C | 104 | 9.690 | 46.810 | −2.053 | 1.00 | 43.44 | C |
| ATOM | 5614 | O | GLU | C | 104 | 9.452 | 47.700 | −2.839 | 1.00 | 44.85 | O |
| ATOM | 5615 | N | ALA | C | 105 | 9.966 | 47.023 | −0.771 | 1.00 | 48.21 | N |
| ATOM | 5617 | CA | ALA | C | 105 | 9.926 | 48.343 | −0.170 | 1.00 | 47.91 | C |
| ATOM | 5619 | CB | ALA | C | 105 | 10.058 | 48.212 | 1.307 | 1.00 | 47.96 | C |
| ATOM | 5623 | C | ALA | C | 105 | 11.096 | 49.133 | −0.720 | 1.00 | 58.84 | C |
| ATOM | 5624 | O | ALA | C | 105 | 12.183 | 48.612 | −0.916 | 1.00 | 51.13 | O |
| ATOM | 5625 | N | PRO | C | 106 | 10.902 | 50.422 | −0.926 | 1.00 | 76.18 | N |
| ATOM | 5626 | CA | PRO | C | 106 | 11.961 | 51.232 | −1.527 | 1.00 | 82.20 | C |
| ATOM | 5628 | CB | PRO | C | 106 | 11.242 | 52.539 | −1.874 | 1.00 | 82.99 | C |
| ATOM | 5631 | CG | PRO | C | 106 | 9.791 | 52.280 | −1.610 | 1.00 | 82.61 | C |
| ATOM | 5634 | CD | PRO | C | 106 | 9.746 | 51.238 | −0.520 | 1.00 | 78.04 | C |
| ATOM | 5637 | C | PRO | C | 106 | 13.103 | 51.449 | −0.521 | 1.00 | 85.07 | C |
| ATOM | 5638 | O | PRO | C | 106 | 14.248 | 51.620 | −0.973 | 1.00 | 86.31 | O |
| ATOM | 5639 | N | ASN | C | 107 | 12.797 | 51.349 | 0.782 | 1.00 | 83.85 | N |
| ATOM | 5641 | CA | ASN | C | 107 | 13.802 | 51.239 | 1.856 | 1.00 | 84.06 | C |
| ATOM | 5643 | CB | ASN | C | 107 | 13.151 | 51.357 | 3.268 | 1.00 | 82.73 | C |
| ATOM | 5646 | CG | ASN | C | 107 | 12.764 | 50.000 | 3.939 | 1.00 | 82.33 | C |
| ATOM | 5647 | OD1 | ASN | C | 107 | 11.662 | 49.823 | 4.474 | 1.00 | 77.63 | O |
| ATOM | 5648 | ND2 | ASN | C | 107 | 13.733 | 49.126 | 4.073 | 1.00 | 70.10 | N |
| ATOM | 5651 | C | ASN | C | 107 | 14.982 | 50.226 | 1.776 | 1.00 | 84.88 | C |
| ATOM | 5652 | O | ASN | C | 107 | 15.854 | 50.297 | 2.623 | 1.00 | 85.78 | O |
| ATOM | 5653 | N | GLN | C | 108 | 15.110 | 49.398 | 0.729 | 1.00 | 84.60 | N |
| ATOM | 5655 | CA | GLN | C | 108 | 15.142 | 47.921 | 0.835 | 1.00 | 81.92 | C |
| ATOM | 5657 | CB | GLN | C | 108 | 16.462 | 47.378 | 0.315 | 1.00 | 77.10 | C |
| ATOM | 5660 | CG | GLN | C | 108 | 16.279 | 46.550 | −0.956 | 1.00 | 93.83 | C |
| ATOM | 5663 | CD | GLN | C | 108 | 16.307 | 47.358 | −2.267 | 1.00 | 98.09 | C |
| ATOM | 5664 | OE1 | GLN | C | 108 | 17.247 | 48.114 | −2.526 | 1.00 | 99.94 | O |
| ATOM | 5665 | NE2 | GLN | C | 108 | 15.320 | 47.123 | −3.130 | 1.00 | 92.29 | N |
| ATOM | 5668 | C | GLN | C | 108 | 14.707 | 47.257 | 2.169 | 1.00 | 80.25 | C |
| ATOM | 5669 | O | GLN | C | 108 | 13.810 | 47.768 | 2.858 | 1.00 | 85.77 | O |
| ATOM | 5670 | N | GLU | C | 109 | 15.240 | 46.090 | 2.523 | 1.00 | 70.74 | N |
| ATOM | 5672 | CA | GLU | C | 109 | 14.985 | 45.561 | 3.867 | 1.00 | 65.05 | C |
| ATOM | 5674 | CB | GLU | C | 109 | 14.695 | 46.673 | 4.863 | 1.00 | 72.51 | C |
| ATOM | 5677 | CG | GLU | C | 109 | 15.807 | 46.927 | 5.863 | 1.00 | 94.02 | C |
| ATOM | 5680 | CD | GLU | C | 109 | 17.030 | 47.568 | 5.216 | 1.00 | 112.06 | C |
| ATOM | 5681 | OE1 | GLU | C | 109 | 17.288 | 48.747 | 5.565 | 1.00 | 96.87 | O |
| ATOM | 5682 | OE2 | GLU | C | 109 | 17.714 | 46.908 | 4.375 | 1.00 | 110.35 | O |
| ATOM | 5683 | C | GLU | C | 109 | 13.781 | 44.664 | 3.927 | 1.00 | 56.07 | C |
| ATOM | 5684 | O | GLU | C | 109 | 13.868 | 43.560 | 4.445 | 1.00 | 65.79 | O |
| ATOM | 5685 | N | LYS | C | 110 | 12.647 | 45.151 | 3.440 | 1.00 | 47.20 | N |
| ATOM | 5687 | CA | LYS | C | 110 | 11.454 | 44.319 | 3.320 | 1.00 | 42.78 | C |
| ATOM | 5689 | CB | LYS | C | 110 | 10.186 | 45.063 | 3.739 | 1.00 | 44.79 | C |
| ATOM | 5692 | CG | LYS | C | 110 | 9.025 | 44.105 | 4.231 | 1.00 | 49.57 | C |
| ATOM | 5695 | CD | LYS | C | 110 | 7.838 | 44.843 | 4.883 | 1.00 | 21.90 | C |
| ATOM | 5698 | CE | LYS | C | 110 | 6.595 | 44.002 | 5.143 | 1.00 | 42.63 | C |
| ATOM | 5701 | NZ | LYS | C | 110 | 5.347 | 44.827 | 5.430 | 1.00 | 34.29 | N |
| ATOM | 5705 | C | LYS | C | 110 | 11.244 | 43.849 | 1.910 | 1.00 | 38.22 | C |
| ATOM | 5706 | O | LYS | C | 110 | 11.258 | 44.630 | 0.988 | 1.00 | 45.19 | O |
| ATOM | 5707 | N | VAL | C | 111 | 11.019 | 42.561 | 1.743 | 1.00 | 37.19 | N |
| ATOM | 5709 | CA | VAL | C | 111 | 10.502 | 42.072 | 0.493 | 1.00 | 40.84 | C |
| ATOM | 5711 | CB | VAL | C | 111 | 11.554 | 41.448 | −0.401 | 1.00 | 40.70 | C |
| ATOM | 5713 | CG1 | VAL | C | 111 | 10.855 | 40.856 | −1.619 | 1.00 | 38.27 | C |
| ATOM | 5717 | CG2 | VAL | C | 111 | 12.583 | 42.469 | −0.774 | 1.00 | 33.95 | C |
| ATOM | 5721 | C | VAL | C | 111 | 9.544 | 40.945 | 0.736 | 1.00 | 47.70 | C |
| ATOM | 5722 | O | VAL | C | 111 | 9.889 | 39.929 | 1.353 | 1.00 | 45.78 | O |
| ATOM | 5723 | N | SER | C | 112 | 8.370 | 41.133 | 0.150 | 1.00 | 49.00 | N |
| ATOM | 5725 | CA | SER | C | 112 | 7.265 | 40.223 | 0.293 | 1.00 | 45.62 | C |
| ATOM | 5727 | CB | SER | C | 112 | 6.033 | 41.009 | 0.737 | 1.00 | 50.64 | C |
| ATOM | 5730 | OG | SER | C | 112 | 6.295 | 41.762 | 1.899 | 1.00 | 36.57 | O |
| ATOM | 5732 | C | SER | C | 112 | 7.017 | 39.570 | −1.064 | 1.00 | 49.17 | C |
| ATOM | 5733 | O | SER | C | 112 | 7.278 | 40.126 | −2.126 | 1.00 | 51.44 | O |
| ATOM | 5734 | N | ASP | C | 113 | 6.463 | 38.372 | −1.000 | 1.00 | 50.35 | N |
| ATOM | 5736 | CA | ASP | C | 113 | 6.538 | 37.406 | −2.074 | 1.00 | 45.57 | C |
| ATOM | 5738 | CB | ASP | C | 113 | 7.843 | 36.649 | −1.857 | 1.00 | 49.49 | C |
| ATOM | 5741 | CG | ASP | C | 113 | 8.273 | 35.898 | −3.051 | 1.00 | 60.89 | C |
| ATOM | 5742 | OD1 | ASP | C | 113 | 7.682 | 34.813 | −3.262 | 1.00 | 98.63 | O |
| ATOM | 5743 | OD2 | ASP | C | 113 | 9.161 | 36.336 | −3.822 | 1.00 | 83.44 | O |
| ATOM | 5744 | C | ASP | C | 113 | 5.271 | 36.527 | −1.918 | 1.00 | 43.03 | C |
| ATOM | 5745 | O | ASP | C | 113 | 5.222 | 35.617 | −1.092 | 1.00 | 43.71 | O |
| ATOM | 5746 | N | TYR | C | 114 | 4.188 | 36.909 | −2.594 | 1.00 | 37.58 | N |
| ATOM | 5748 | CA | TYR | C | 114 | 2.916 | 36.203 | −2.481 | 1.00 | 36.03 | C |
| ATOM | 5750 | CB | TYR | C | 114 | 1.802 | 37.168 | −2.174 | 1.00 | 34.06 | C |
| ATOM | 5753 | CG | TYR | C | 114 | 1.946 | 37.799 | −0.842 | 1.00 | 28.88 | C |
| ATOM | 5754 | CD1 | TYR | C | 114 | 2.520 | 39.041 | −0.708 | 1.00 | 27.41 | C |
| ATOM | 5756 | CE1 | TYR | C | 114 | 2.633 | 39.639 | 0.540 | 1.00 | 27.59 | C |
| ATOM | 5758 | CZ | TYR | C | 114 | 2.131 | 39.002 | 1.646 | 1.00 | 25.31 | C |
| ATOM | 5759 | OH | TYR | C | 114 | 2.223 | 39.618 | 2.872 | 1.00 | 26.31 | O |
| ATOM | 5761 | CE2 | TYR | C | 114 | 1.519 | 37.777 | 1.512 | 1.00 | 32.96 | C |
| ATOM | 5763 | CD2 | TYR | C | 114 | 1.404 | 37.202 | 0.273 | 1.00 | 24.64 | C |
| ATOM | 5765 | C | TYR | C | 114 | 2.571 | 35.460 | −3.764 | 1.00 | 36.27 | C |
| ATOM | 5766 | O | TYR | C | 114 | 3.021 | 35.806 | −4.850 | 1.00 | 37.69 | O |
| ATOM | 5767 | N | GLU | C | 115 | 1.761 | 34.428 | −3.627 | 1.00 | 33.23 | N |
| ATOM | 5769 | CA | GLU | C | 115 | 1.439 | 33.564 | −4.748 | 1.00 | 35.37 | C |
| ATOM | 5771 | CB | GLU | C | 115 | 2.229 | 32.276 | −4.661 | 1.00 | 36.69 | C |
| ATOM | 5774 | CG | GLU | C | 115 | 1.616 | 31.119 | −5.427 | 1.00 | 50.20 | C |
| ATOM | 5777 | CD | GLU | C | 115 | 2.646 | 30.059 | −5.751 | 1.00 | 62.88 | C |
| ATOM | 5778 | OE1 | GLU | C | 115 | 3.767 | 30.510 | −6.073 | 1.00 | 59.89 | O |
| ATOM | 5779 | OE2 | GLU | C | 115 | 2.341 | 28.833 | −5.639 | 1.00 | 58.76 | O |
| ATOM | 5780 | C | GLU | C | 115 | −0.015 | 33.172 | −4.643 | 1.00 | 32.83 | C |
| ATOM | 5781 | O | GLU | C | 115 | −0.400 | 32.607 | −3.645 | 1.00 | 26.45 | O |
| ATOM | 5782 | N | MET | C | 116 | −0.790 | 33.458 | −5.680 | 1.00 | 38.06 | N |
| ATOM | 5784 | CA | MET | C | 116 | −2.242 | 33.569 | −5.598 | 1.00 | 37.23 | C |
| ATOM | 5786 | CB | MET | C | 116 | −2.646 | 34.957 | −6.133 | 1.00 | 34.13 | C |
| ATOM | 5789 | CG | MET | C | 116 | −4.123 | 35.093 | −6.373 | 1.00 | 48.84 | C |
| ATOM | 5792 | SD | MET | C | 116 | −4.857 | 36.757 | −6.632 | 1.00 | 43.68 | S |
| ATOM | 5793 | CE | MET | C | 116 | −4.582 | 36.941 | −8.420 | 1.00 | 41.93 | C |
| ATOM | 5797 | C | MET | C | 116 | −2.725 | 32.480 | −6.555 | 1.00 | 40.76 | C |
| ATOM | 5798 | O | MET | C | 116 | −2.235 | 32.412 | −7.697 | 1.00 | 37.98 | O |
| ATOM | 5799 | N | LYS | C | 117 | −3.583 | 31.578 | −6.081 | 1.00 | 40.07 | N |
| ATOM | 5801 | CA | LYS | C | 117 | −4.124 | 30.507 | −6.935 | 1.00 | 46.54 | C |
| ATOM | 5803 | CB | LYS | C | 117 | −4.859 | 29.438 | −6.119 | 1.00 | 52.05 | C |
| ATOM | 5806 | CG | LYS | C | 117 | −4.055 | 28.162 | −5.768 | 1.00 | 74.54 | C |
| ATOM | 5809 | CD | LYS | C | 117 | −4.437 | 27.628 | −4.361 | 1.00 | 89.71 | C |
| ATOM | 5812 | CE | LYS | C | 117 | −3.458 | 26.590 | −3.808 | 1.00 | 90.41 | C |
| ATOM | 5815 | NZ | LYS | C | 117 | −4.105 | 25.258 | −3.560 | 1.00 | 93.08 | N |
| ATOM | 5819 | C | LYS | C | 117 | −5.122 | 31.129 | −7.909 | 1.00 | 48.01 | C |
| ATOM | 5820 | O | LYS | C | 117 | −5.815 | 32.080 | −7.561 | 1.00 | 53.01 | O |
| ATOM | 5821 | N | LEU | C | 118 | −5.165 | 30.631 | −9.139 | 1.00 | 47.75 | N |
| ATOM | 5823 | CA | LEU | C | 118 | −6.036 | 31.181 | −10.173 | 1.00 | 42.82 | C |
| ATOM | 5825 | CB | LEU | C | 118 | −5.347 | 31.288 | −11.517 | 1.00 | 39.40 | C |
| ATOM | 5828 | CG | LEU | C | 118 | −4.284 | 32.389 | −11.464 | 1.00 | 39.67 | C |
| ATOM | 5830 | CD1 | LEU | C | 118 | −3.392 | 32.322 | −12.665 | 1.00 | 29.87 | C |
| ATOM | 5834 | CD2 | LEU | C | 118 | −4.876 | 33.776 | −11.350 | 1.00 | 35.56 | C |
| ATOM | 5838 | C | LEU | C | 118 | −7.181 | 30.249 | −10.321 | 1.00 | 43.30 | C |
| ATOM | 5839 | O | LEU | C | 118 | −7.375 | 29.384 | −9.490 | 1.00 | 50.05 | O |
| ATOM | 5840 | N | MET | C | 119 | −8.048 | 30.529 | −11.270 | 1.00 | 51.13 | N |
| ATOM | 5842 | CA | MET | C | 119 | −9.381 | 29.945 | −11.188 | 1.00 | 53.38 | C |
| ATOM | 5844 | CB | MET | C | 119 | −10.290 | 30.670 | −10.179 | 1.00 | 36.61 | C |
| ATOM | 5847 | CG | MET | C | 119 | −11.093 | 31.807 | −10.758 | 1.00 | 55.61 | C |
| ATOM | 5850 | SD | MET | C | 119 | −12.457 | 32.272 | −9.653 | 1.00 | 81.48 | S |
| ATOM | 5851 | CE | MET | C | 119 | −11.733 | 31.623 | −7.982 | 1.00 | 80.36 | C |
| ATOM | 5855 | C | MET | C | 119 | −9.879 | 29.961 | −12.615 | 1.00 | 53.90 | C |
| ATOM | 5856 | O | MET | C | 119 | −9.547 | 30.898 | −13.340 | 1.00 | 56.57 | O |
| ATOM | 5857 | N | ASP | C | 120 | −10.480 | 28.848 | −13.039 | 1.00 | 57.35 | N |
| ATOM | 5859 | CA | ASP | C | 120 | −11.087 | 28.726 | −14.367 | 1.00 | 67.46 | C |
| ATOM | 5861 | CB | ASP | C | 120 | −11.437 | 27.260 | −14.673 | 1.00 | 66.45 | C |
| ATOM | 5864 | CG | ASP | C | 120 | −10.267 | 26.509 | −15.249 | 1.00 | 84.19 | C |
| ATOM | 5865 | OD1 | ASP | C | 120 | −9.811 | 26.891 | −16.352 | 1.00 | 107.84 | O |
| ATOM | 5866 | OD2 | ASP | C | 120 | −9.692 | 25.585 | −14.633 | 1.00 | 107.20 | O |
| ATOM | 5867 | C | ASP | C | 120 | −12.352 | 29.577 | −14.431 | 1.00 | 62.04 | C |
| ATOM | 5868 | O | ASP | C | 120 | −13.184 | 29.456 | −13.550 | 1.00 | 58.78 | O |
| ATOM | 5869 | N | LEU | C | 121 | −12.514 | 30.413 | −15.451 | 1.00 | 60.08 | N |
| ATOM | 5871 | CA | LEU | C | 121 | −13.858 | 30.788 | −15.856 | 1.00 | 63.70 | C |
| ATOM | 5873 | CB | LEU | C | 121 | −14.291 | 32.089 | −15.198 | 1.00 | 66.66 | C |
| ATOM | 5876 | CG | LEU | C | 121 | −13.176 | 32.908 | −14.545 | 1.00 | 69.12 | C |
| ATOM | 5878 | CD1 | LEU | C | 121 | −12.760 | 34.000 | −15.500 | 1.00 | 90.06 | C |
| ATOM | 5882 | CD2 | LEU | C | 121 | −13.594 | 33.519 | −13.235 | 1.00 | 62.30 | C |
| ATOM | 5886 | C | LEU | C | 121 | −13.897 | 30.946 | −17.345 | 1.00 | 67.30 | C |
| ATOM | 5887 | O | LEU | C | 121 | −12.882 | 31.230 | −17.961 | 1.00 | 63.30 | O |
| ATOM | 5888 | N | ASP | C | 122 | −15.072 | 30.756 | −17.927 | 1.00 | 79.87 | N |
| ATOM | 5890 | CA | ASP | C | 122 | −15.295 | 31.247 | −19.288 | 1.00 | 87.51 | C |
| ATOM | 5892 | CB | ASP | C | 122 | −15.881 | 30.174 | −20.228 | 1.00 | 90.36 | C |
| ATOM | 5895 | CG | ASP | C | 122 | −17.366 | 29.916 | −19.980 | 1.00 | 100.85 | C |
| ATOM | 5896 | OD1 | ASP | C | 122 | −18.169 | 30.062 | −20.935 | 1.00 | 98.66 | O |
| ATOM | 5897 | OD2 | ASP | C | 122 | −17.815 | 29.573 | −18.860 | 1.00 | 95.74 | O |
| ATOM | 5898 | C | ASP | C | 122 | −16.154 | 32.513 | −19.263 | 1.00 | 80.36 | C |
| ATOM | 5899 | O | ASP | C | 122 | −17.069 | 32.665 | −18.448 | 1.00 | 76.49 | O |
| ATOM | 5900 | N | VAL | C | 123 | −15.805 | 33.413 | −20.170 | 1.00 | 71.74 | N |
| ATOM | 5902 | CA | VAL | C | 123 | −16.357 | 34.742 | −20.233 | 1.00 | 72.12 | C |
| ATOM | 5904 | CB | VAL | C | 123 | −15.387 | 35.733 | −19.549 | 1.00 | 79.50 | C |
| ATOM | 5906 | CG1 | VAL | C | 123 | −15.893 | 36.163 | −18.153 | 1.00 | 82.19 | C |
| ATOM | 5910 | CG2 | VAL | C | 123 | −13.965 | 35.144 | −19.489 | 1.00 | 72.70 | C |
| ATOM | 5914 | C | VAL | C | 123 | −16.413 | 35.031 | −21.728 | 1.00 | 70.04 | C |
| ATOM | 5915 | O | VAL | C | 123 | −15.407 | 34.907 | −22.419 | 1.00 | 67.97 | O |
| ATOM | 5916 | N | GLU | C | 124 | −17.575 | 35.379 | −22.263 | 1.00 | 72.36 | N |
| ATOM | 5918 | CA | GLU | C | 124 | −17.584 | 36.018 | −23.574 | 1.00 | 73.99 | C |
| ATOM | 5920 | CB | GLU | C | 124 | −18.926 | 35.851 | −24.312 | 1.00 | 78.34 | C |
| ATOM | 5923 | CG | GLU | C | 124 | −19.965 | 34.916 | −23.685 | 1.00 | 89.60 | C |
| ATOM | 5926 | CD | GLU | C | 124 | −20.900 | 34.262 | −24.708 | 1.00 | 101.35 | C |
| ATOM | 5927 | OE1 | GLU | C | 124 | −20.729 | 33.062 | −25.035 | 1.00 | 99.41 | O |
| ATOM | 5928 | OE2 | GLU | C | 124 | −21.836 | 34.935 | −25.186 | 1.00 | 93.30 | O |
| ATOM | 5929 | C | GLU | C | 124 | −17.252 | 37.498 | −23.356 | 1.00 | 67.28 | C |
| ATOM | 5930 | O | GLU | C | 124 | −17.978 | 38.220 | −22.678 | 1.00 | 70.49 | O |
| ATOM | 5931 | N | GLN | C | 125 | −16.130 | 37.937 | −23.908 | 1.00 | 59.49 | N |
| ATOM | 5933 | CA | GLN | C | 125 | −15.926 | 39.347 | −24.185 | 1.00 | 55.48 | C |
| ATOM | 5935 | CB | GLN | C | 125 | −14.565 | 39.544 | −24.829 | 1.00 | 54.64 | C |
| ATOM | 5938 | CG | GLN | C | 125 | −13.650 | 40.524 | −24.098 | 1.00 | 64.32 | C |
| ATOM | 5941 | CD | GLN | C | 125 | −12.982 | 41.491 | −25.068 | 1.00 | 77.01 | C |
| ATOM | 5942 | OE1 | GLN | C | 125 | −12.507 | 41.082 | −26.131 | 1.00 | 89.35 | O |
| ATOM | 5943 | NE2 | GLN | C | 125 | −12.997 | 42.777 | −24.737 | 1.00 | 80.07 | N |
| ATOM | 5946 | C | GLN | C | 125 | −17.046 | 39.938 | −25.067 | 1.00 | 54.81 | C |
| ATOM | 5947 | O | GLN | C | 125 | −17.355 | 39.432 | −26.155 | 1.00 | 51.40 | O |
| ATOM | 5948 | N | LEU | C | 126 | −17.650 | 41.010 | −24.548 | 1.00 | 49.38 | N |
| ATOM | 5950 | CA | LEU | C | 126 | −18.660 | 41.819 | −25.224 | 1.00 | 40.52 | C |
| ATOM | 5952 | CB | LEU | C | 126 | −19.536 | 42.531 | −24.200 | 1.00 | 38.57 | C |
| ATOM | 5955 | CG | LEU | C | 126 | −20.164 | 41.500 | −23.273 | 1.00 | 33.40 | C |
| ATOM | 5957 | CD1 | LEU | C | 126 | −21.082 | 42.106 | −22.267 | 1.00 | 38.00 | C |
| ATOM | 5961 | CD2 | LEU | C | 126 | −20.946 | 40.511 | −24.120 | 1.00 | 58.67 | C |
| ATOM | 5965 | C | LEU | C | 126 | −17.974 | 42.870 | −26.041 | 1.00 | 38.48 | C |
| ATOM | 5966 | O | LEU | C | 126 | −17.050 | 43.496 | −25.544 | 1.00 | 41.55 | O |
| ATOM | 5967 | N | GLY | C | 127 | −18.387 | 43.008 | −27.300 | 1.00 | 43.24 | N |
| ATOM | 5969 | CA | GLY | C | 127 | −17.961 | 44.108 | −28.158 | 1.00 | 45.69 | C |
| ATOM | 5972 | C | GLY | C | 127 | −18.654 | 45.428 | −27.841 | 1.00 | 42.78 | C |
| ATOM | 5973 | O | GLY | C | 127 | −19.872 | 45.536 | −27.765 | 1.00 | 42.17 | O |
| ATOM | 5974 | N | ILE | C | 128 | −17.841 | 46.434 | −27.591 | 1.00 | 42.36 | N |
| ATOM | 5976 | CA | ILE | C | 128 | −18.343 | 47.760 | −27.347 | 1.00 | 47.14 | C |
| ATOM | 5978 | CB | ILE | C | 128 | −17.588 | 48.354 | −26.164 | 1.00 | 53.70 | C |
| ATOM | 5980 | CG1 | ILE | C | 128 | −17.976 | 47.553 | −24.906 | 1.00 | 55.47 | C |
| ATOM | 5983 | CD1 | ILE | C | 128 | −17.040 | 47.778 | −23.729 | 1.00 | 66.65 | C |
| ATOM | 5987 | CG2 | ILE | C | 128 | −17.874 | 49.862 | −26.075 | 1.00 | 46.86 | C |
| ATOM | 5991 | C | ILE | C | 128 | −18.124 | 48.594 | −28.596 | 1.00 | 48.92 | C |
| ATOM | 5992 | O | ILE | C | 128 | −17.013 | 48.738 | −29.098 | 1.00 | 48.45 | O |
| ATOM | 5993 | N | PRO | C | 129 | −19.214 | 49.098 | −29.142 | 1.00 | 47.81 | N |
| ATOM | 5994 | CA | PRO | C | 129 | −19.205 | 49.598 | −30.508 | 1.00 | 43.42 | C |
| ATOM | 5996 | CB | PRO | C | 129 | −20.663 | 49.482 | −30.926 | 1.00 | 44.31 | C |
| ATOM | 5999 | CG | PRO | C | 129 | −21.459 | 49.541 | −29.684 | 1.00 | 42.78 | C |
| ATOM | 6002 | CD | PRO | C | 129 | −20.539 | 49.240 | −28.520 | 1.00 | 55.47 | C |
| ATOM | 6005 | C | PRO | C | 129 | −18.834 | 51.041 | −30.471 | 1.00 | 41.27 | C |
| ATOM | 6006 | O | PRO | C | 129 | −19.070 | 51.705 | −29.482 | 1.00 | 41.22 | O |
| ATOM | 6007 | N | GLU | C | 130 | −18.265 | 51.522 | −31.557 | 1.00 | 47.92 | N |
| ATOM | 6009 | CA | GLU | C | 130 | −17.936 | 52.922 | −31.631 | 1.00 | 49.57 | C |
| ATOM | 6011 | CB | GLU | C | 130 | −17.202 | 53.243 | −32.948 | 1.00 | 54.28 | C |
| ATOM | 6014 | CG | GLU | C | 130 | −16.038 | 54.216 | −32.804 | 1.00 | 57.58 | C |
| ATOM | 6017 | CD | GLU | C | 130 | −14.791 | 53.442 | −32.499 | 1.00 | 66.72 | C |
| ATOM | 6018 | OE1 | GLU | C | 130 | −14.245 | 52.870 | −33.447 | 1.00 | 69.33 | O |
| ATOM | 6019 | OE2 | GLU | C | 130 | −14.430 | 53.304 | −31.312 | 1.00 | 93.50 | O |
| ATOM | 6020 | C | GLU | C | 130 | −19.254 | 53.703 | −31.464 | 1.00 | 51.68 | C |
| ATOM | 6021 | O | GLU | C | 130 | −20.328 | 53.318 | −31.978 | 1.00 | 41.92 | O |
| ATOM | 6022 | N | GLN | C | 131 | −19.150 | 54.811 | −30.731 | 1.00 | 50.73 | N |
| ATOM | 6024 | CA | GLN | C | 131 | −20.321 | 55.613 | −30.432 | 1.00 | 50.75 | C |
| ATOM | 6026 | CB | GLN | C | 131 | −21.077 | 54.837 | −29.359 | 1.00 | 55.93 | C |
| ATOM | 6029 | CG | GLN | C | 131 | −21.817 | 55.595 | −28.297 | 1.00 | 51.93 | C |
| ATOM | 6032 | CD | GLN | C | 131 | −23.298 | 55.354 | −28.512 | 1.00 | 73.42 | C |
| ATOM | 6033 | OE1 | GLN | C | 131 | −23.992 | 56.209 | −29.094 | 1.00 | 49.65 | O |
| ATOM | 6034 | NE2 | GLN | C | 131 | −23.750 | 54.131 | −28.180 | 1.00 | 56.53 | N |
| ATOM | 6037 | C | GLN | C | 131 | −20.055 | 57.080 | −30.060 | 1.00 | 48.57 | C |
| ATOM | 6038 | O | GLN | C | 131 | −19.068 | 57.416 | −29.418 | 1.00 | 47.38 | O |
| ATOM | 6039 | N | GLU | C | 132 | −20.942 | 57.956 | −30.513 | 1.00 | 53.08 | N |
| ATOM | 6041 | CA | GLU | C | 132 | −20.776 | 59.381 | −30.301 | 1.00 | 54.53 | C |
| ATOM | 6043 | CB | GLU | C | 132 | −21.189 | 60.159 | −31.552 | 1.00 | 59.92 | C |
| ATOM | 6046 | CG | GLU | C | 132 | −19.963 | 60.588 | −32.369 | 1.00 | 78.93 | C |
| ATOM | 6049 | CD | GLU | C | 132 | −19.537 | 62.019 | −32.102 | 1.00 | 79.81 | C |
| ATOM | 6050 | OE1 | GLU | C | 132 | −20.168 | 62.911 | −32.689 | 1.00 | 75.79 | O |
| ATOM | 6051 | OE2 | GLU | C | 132 | −18.619 | 62.266 | −31.288 | 1.00 | 98.81 | O |
| ATOM | 6052 | C | GLU | C | 132 | −21.706 | 59.704 | −29.176 | 1.00 | 44.97 | C |
| ATOM | 6053 | O | GLU | C | 132 | −22.847 | 59.279 | −29.191 | 1.00 | 49.37 | O |
| ATOM | 6054 | N | TYR | C | 133 | −21.204 | 60.416 | −28.186 | 1.00 | 43.18 | N |
| ATOM | 6056 | CA | TYR | C | 133 | −22.031 | 60.832 | −27.057 | 1.00 | 42.78 | C |
| ATOM | 6058 | CB | TYR | C | 133 | −21.293 | 60.591 | −25.720 | 1.00 | 40.67 | C |
| ATOM | 6061 | CG | TYR | C | 133 | −20.978 | 59.118 | −25.465 | 1.00 | 37.51 | C |
| ATOM | 6062 | CD1 | TYR | C | 133 | −19.775 | 58.574 | −25.851 | 1.00 | 27.65 | C |
| ATOM | 6064 | CE1 | TYR | C | 133 | −19.486 | 57.257 | −25.622 | 1.00 | 36.37 | C |
| ATOM | 6066 | CZ | TYR | C | 133 | −20.408 | 56.413 | −25.060 | 1.00 | 36.75 | C |
| ATOM | 6067 | OH | TYR | C | 133 | −20.083 | 55.078 | −24.875 | 1.00 | 53.67 | O |
| ATOM | 6069 | CE2 | TYR | C | 133 | −21.637 | 56.894 | −24.734 | 1.00 | 45.05 | C |
| ATOM | 6071 | CD2 | TYR | C | 133 | −21.916 | 58.249 | −24.922 | 1.00 | 57.70 | C |
| ATOM | 6073 | C | TYR | C | 133 | −22.481 | 62.279 | −27.201 | 1.00 | 35.74 | C |
| ATOM | 6074 | O | TYR | C | 133 | −21.742 | 63.133 | −27.647 | 1.00 | 49.39 | O |
| ATOM | 6075 | N | SER | C | 134 | −23.706 | 62.581 | −26.824 | 1.00 | 35.75 | N |
| ATOM | 6077 | CA | SER | C | 134 | −24.215 | 63.927 | −27.029 | 1.00 | 41.24 | C |
| ATOM | 6079 | CB | SER | C | 134 | −25.726 | 63.966 | −26.906 | 1.00 | 44.71 | C |
| ATOM | 6082 | OG | SER | C | 134 | −26.283 | 62.833 | −27.524 | 1.00 | 37.21 | O |
| ATOM | 6084 | C | SER | C | 134 | −23.675 | 64.907 | −26.033 | 1.00 | 41.46 | C |
| ATOM | 6085 | O | SER | C | 134 | −23.623 | 66.092 | −26.317 | 1.00 | 47.23 | O |
| ATOM | 6086 | N | CYS | C | 135 | −23.277 | 64.395 | −24.872 | 1.00 | 50.34 | N |
| ATOM | 6088 | CA | CYS | C | 135 | −22.810 | 65.226 | −23.767 | 1.00 | 48.12 | C |
| ATOM | 6090 | CB | CYS | C | 135 | −23.946 | 65.402 | −22.797 | 1.00 | 39.42 | C |
| ATOM | 6093 | SG | CYS | C | 135 | −24.427 | 67.095 | −22.639 | 1.00 | 65.66 | S |
| ATOM | 6094 | C | CYS | C | 135 | −21.699 | 64.510 | −23.049 | 1.00 | 46.20 | C |
| ATOM | 6095 | O | CYS | C | 135 | −21.879 | 63.364 | −22.648 | 1.00 | 51.76 | O |
| ATOM | 6096 | N | VAL | C | 136 | −20.556 | 65.169 | −22.891 | 1.00 | 46.05 | N |
| ATOM | 6098 | CA | VAL | C | 136 | −19.460 | 64.598 | −22.117 | 1.00 | 34.69 | C |
| ATOM | 6100 | CB | VAL | C | 136 | −18.266 | 64.291 | −22.989 | 1.00 | 25.84 | C |
| ATOM | 6102 | CG1 | VAL | C | 136 | −17.225 | 63.565 | −22.211 | 1.00 | 39.25 | C |
| ATOM | 6106 | CG2 | VAL | C | 136 | −18.693 | 63.511 | −24.214 | 1.00 | 39.16 | C |
| ATOM | 6110 | C | VAL | C | 136 | −19.083 | 65.597 | −21.051 | 1.00 | 39.63 | C |
| ATOM | 6111 | O | VAL | C | 136 | −18.691 | 66.729 | −21.349 | 1.00 | 42.85 | O |
| ATOM | 6112 | N | VAL | C | 137 | −19.318 | 65.211 | −19.802 | 1.00 | 42.26 | N |
| ATOM | 6114 | CA | VAL | C | 137 | −19.087 | 66.131 | −18.695 | 1.00 | 45.08 | C |
| ATOM | 6116 | CB | VAL | C | 137 | −20.209 | 66.084 | −17.677 | 1.00 | 36.98 | C |
| ATOM | 6118 | CG1 | VAL | C | 137 | −19.927 | 67.018 | −16.534 | 1.00 | 50.77 | C |
| ATOM | 6122 | CG2 | VAL | C | 137 | −21.502 | 66.447 | −18.334 | 1.00 | 58.37 | C |
| ATOM | 6126 | C | VAL | C | 137 | −17.795 | 65.725 | −18.016 | 1.00 | 42.53 | C |
| ATOM | 6127 | O | VAL | C | 137 | −17.622 | 64.595 | −17.667 | 1.00 | 37.19 | O |
| ATOM | 6128 | N | LYS | C | 138 | −16.876 | 66.657 | −17.868 | 1.00 | 48.83 | N |
| ATOM | 6130 | CA | LYS | C | 138 | −15.620 | 66.434 | −17.177 | 1.00 | 45.94 | C |
| ATOM | 6132 | CB | LYS | C | 138 | −14.487 | 66.940 | −18.087 | 1.00 | 47.37 | C |
| ATOM | 6135 | CG | LYS | C | 138 | −13.138 | 66.328 | −17.855 | 1.00 | 70.97 | C |
| ATOM | 6138 | CD | LYS | C | 138 | −12.028 | 67.399 | −17.848 | 1.00 | 97.62 | C |
| ATOM | 6141 | CE | LYS | C | 138 | −10.800 | 67.014 | −16.992 | 1.00 | 99.08 | C |
| ATOM | 6144 | NZ | LYS | C | 138 | −9.490 | 67.266 | −17.675 | 1.00 | 105.03 | N |
| ATOM | 6148 | C | LYS | C | 138 | −15.725 | 67.258 | −15.879 | 1.00 | 40.14 | C |
| ATOM | 6149 | O | LYS | C | 138 | −15.893 | 68.474 | −15.906 | 1.00 | 37.50 | O |
| ATOM | 6150 | N | MET | C | 139 | −15.638 | 66.565 | −14.752 | 1.00 | 32.14 | N |
| ATOM | 6152 | CA | MET | C | 139 | −15.788 | 67.156 | −13.446 | 1.00 | 27.50 | C |
| ATOM | 6154 | CB | MET | C | 139 | −17.211 | 67.025 | −12.991 | 1.00 | 26.71 | C |
| ATOM | 6157 | CG | MET | C | 139 | −17.740 | 65.625 | −12.785 | 1.00 | 31.25 | C |
| ATOM | 6160 | SD | MET | C | 139 | −19.420 | 65.703 | −11.957 | 1.00 | 36.64 | S |
| ATOM | 6161 | CE | MET | C | 139 | −20.033 | 64.525 | −12.850 | 1.00 | 44.65 | C |
| ATOM | 6165 | C | MET | C | 139 | −14.928 | 66.505 | −12.378 | 1.00 | 34.98 | C |
| ATOM | 6166 | O | MET | C | 139 | −14.320 | 65.441 | −12.565 | 1.00 | 34.25 | O |
| ATOM | 6167 | N | PRO | C | 140 | −14.882 | 67.147 | −11.224 | 1.00 | 31.93 | N |
| ATOM | 6168 | CA | PRO | C | 140 | −14.064 | 66.629 | −10.124 | 1.00 | 30.93 | C |
| ATOM | 6170 | CB | PRO | C | 140 | −14.176 | 67.714 | −9.048 | 1.00 | 24.00 | C |
| ATOM | 6173 | CG | PRO | C | 140 | −14.945 | 68.877 | −9.626 | 1.00 | 32.95 | C |
| ATOM | 6176 | CD | PRO | C | 140 | −15.584 | 68.400 | −10.896 | 1.00 | 31.97 | C |
| ATOM | 6179 | C | PRO | C | 140 | −14.607 | 65.293 | −9.598 | 1.00 | 36.54 | C |
| ATOM | 6180 | O | PRO | C | 140 | −15.818 | 65.155 | −9.489 | 1.00 | 39.32 | O |
| ATOM | 6181 | N | SER | C | 141 | −13.764 | 64.310 | −9.291 | 1.00 | 40.99 | N |
| ATOM | 6183 | CA | SER | C | 141 | −14.283 | 62.982 | −8.934 | 1.00 | 42.03 | C |
| ATOM | 6185 | CB | SER | C | 141 | −13.240 | 61.897 | −9.136 | 1.00 | 41.07 | C |
| ATOM | 6188 | OG | SER | C | 141 | −12.070 | 62.173 | −8.385 | 1.00 | 49.29 | O |
| ATOM | 6190 | C | SER | C | 141 | −14.781 | 62.908 | −7.503 | 1.00 | 44.53 | C |
| ATOM | 6191 | O | SER | C | 141 | −15.768 | 62.233 | −7.214 | 1.00 | 46.59 | O |
| ATOM | 6192 | N | GLY | C | 142 | −14.116 | 63.624 | −6.609 | 1.00 | 43.13 | N |
| ATOM | 6194 | CA | GLY | C | 142 | −14.728 | 63.947 | −5.340 | 1.00 | 43.17 | C |
| ATOM | 6197 | C | GLY | C | 142 | −16.170 | 64.400 | −5.429 | 1.00 | 48.59 | C |
| ATOM | 6198 | O | GLY | C | 142 | −17.033 | 63.928 | −4.671 | 1.00 | 50.71 | O |
| ATOM | 6199 | N | GLU | C | 143 | −16.454 | 65.358 | −6.311 | 1.00 | 49.24 | N |
| ATOM | 6201 | CA | GLU | C | 143 | −17.782 | 65.985 | −6.285 | 1.00 | 43.32 | C |
| ATOM | 6203 | CB | GLU | C | 143 | −17.829 | 67.248 | −7.146 | 1.00 | 42.91 | C |
| ATOM | 6206 | CG | GLU | C | 143 | −19.136 | 68.002 | −7.018 | 1.00 | 55.75 | C |
| ATOM | 6209 | CD | GLU | C | 143 | −19.274 | 68.611 | −5.648 | 1.00 | 59.03 | C |
| ATOM | 6210 | OE1 | GLU | C | 143 | −18.338 | 69.323 | −5.245 | 1.00 | 62.01 | O |
| ATOM | 6211 | OE2 | GLU | C | 143 | −20.300 | 68.356 | −4.986 | 1.00 | 70.96 | O |
| ATOM | 6212 | C | GLU | C | 143 | −18.809 | 64.979 | −6.785 | 1.00 | 33.37 | C |
| ATOM | 6213 | O | GLU | C | 143 | −19.856 | 64.816 | −6.193 | 1.00 | 27.73 | O |
| ATOM | 6214 | N | PHE | C | 144 | −18.462 | 64.261 | −7.848 | 1.00 | 32.79 | N |
| ATOM | 6216 | CA | PHE | C | 144 | −19.333 | 63.239 | −8.377 | 1.00 | 34.31 | C |
| ATOM | 6218 | CB | PHE | C | 144 | −18.703 | 62.536 | −9.580 | 1.00 | 35.30 | C |
| ATOM | 6221 | CG | PHE | C | 144 | −19.628 | 61.526 | −10.230 | 1.00 | 35.17 | C |
| ATOM | 6222 | CD1 | PHE | C | 144 | −20.886 | 61.898 | −10.674 | 1.00 | 41.66 | C |
| ATOM | 6224 | CE1 | PHE | C | 144 | −21.761 | 60.972 | −11.232 | 1.00 | 37.18 | C |
| ATOM | 6226 | CZ | PHE | C | 144 | −21.371 | 59.654 | −11.328 | 1.00 | 42.29 | C |
| ATOM | 6228 | CE2 | PHE | C | 144 | −20.093 | 59.286 | −10.943 | 1.00 | 38.55 | C |
| ATOM | 6230 | CD2 | PHE | C | 144 | −19.249 | 60.205 | −10.369 | 1.00 | 26.01 | C |
| ATOM | 6232 | C | PHE | C | 144 | −19.703 | 62.269 | −7.274 | 1.00 | 32.10 | C |
| ATOM | 6233 | O | PHE | C | 144 | −20.873 | 62.144 | −6.950 | 1.00 | 35.71 | O |
| ATOM | 6234 | N | ALA | C | 145 | −18.709 | 61.688 | −6.608 | 1.00 | 38.34 | N |
| ATOM | 6236 | CA | ALA | C | 145 | −18.970 | 60.845 | −5.434 | 1.00 | 37.59 | C |
| ATOM | 6238 | CB | ALA | C | 145 | −17.716 | 60.190 | −4.896 | 1.00 | 38.71 | C |
| ATOM | 6242 | C | ALA | C | 145 | −19.771 | 61.474 | −4.315 | 1.00 | 32.52 | C |
| ATOM | 6243 | O | ALA | C | 145 | −20.597 | 60.769 | −3.734 | 1.00 | 38.91 | O |
| ATOM | 6244 | N | ARG | C | 146 | −19.590 | 62.770 | −4.038 | 1.00 | 36.80 | N |
| ATOM | 6246 | CA | ARG | C | 146 | −20.428 | 63.482 | −3.043 | 1.00 | 35.33 | C |
| ATOM | 6248 | CB | ARG | C | 146 | −19.942 | 64.916 | −2.899 | 1.00 | 35.21 | C |
| ATOM | 6251 | CG | ARG | C | 146 | −20.215 | 65.691 | −1.609 | 1.00 | 48.97 | C |
| ATOM | 6254 | CD | ARG | C | 146 | −19.362 | 67.014 | −1.513 | 1.00 | 64.45 | C |
| ATOM | 6257 | NE | ARG | C | 146 | −17.986 | 66.854 | −0.993 | 1.00 | 71.50 | N |
| ATOM | 6259 | CZ | ARG | C | 146 | −16.840 | 66.949 | −1.698 | 1.00 | 82.34 | C |
| ATOM | 6260 | NH1 | ARG | C | 146 | −16.823 | 67.169 | −3.008 | 1.00 | 79.55 | N |
| ATOM | 6263 | NH2 | ARG | C | 146 | −15.669 | 66.785 | −1.093 | 1.00 | 85.28 | N |
| ATOM | 6266 | C | ARG | C | 146 | −21.881 | 63.526 | −3.504 | 1.00 | 36.91 | C |
| ATOM | 6267 | O | ARG | C | 146 | −22.783 | 63.265 | −2.715 | 1.00 | 40.84 | O |
| ATOM | 6268 | N | ILE | C | 147 | −22.111 | 63.844 | −4.778 | 1.00 | 28.81 | N |
| ATOM | 6270 | CA | ILE | C | 147 | −23.468 | 63.902 | −5.270 | 1.00 | 32.71 | C |
| ATOM | 6272 | CB | ILE | C | 147 | −23.532 | 64.400 | −6.718 | 1.00 | 39.84 | C |
| ATOM | 6274 | CG1 | ILE | C | 147 | −23.673 | 65.902 | −6.744 | 1.00 | 26.63 | C |
| ATOM | 6277 | CD1 | ILE | C | 147 | −22.490 | 66.563 | −7.386 | 1.00 | 38.95 | C |
| ATOM | 6281 | CG2 | ILE | C | 147 | −24.751 | 63.846 | −7.474 | 1.00 | 45.90 | C |
| ATOM | 6285 | C | ILE | C | 147 | −24.109 | 62.530 | −5.205 | 1.00 | 37.59 | C |
| ATOM | 6286 | O | ILE | C | 147 | −25.243 | 62.423 | −4.732 | 1.00 | 33.25 | O |
| ATOM | 6287 | N | CYS | C | 148 | −23.455 | 61.509 | −5.757 | 1.00 | 32.48 | N |
| ATOM | 6289 | CA | CYS | C | 148 | −24.063 | 60.185 | −5.695 | 1.00 | 42.80 | C |
| ATOM | 6291 | CB | CYS | C | 148 | −23.187 | 59.165 | −6.407 | 1.00 | 42.70 | C |
| ATOM | 6294 | SG | CYS | C | 148 | −23.140 | 59.594 | −8.120 | 1.00 | 49.48 | S |
| ATOM | 6295 | C | CYS | C | 148 | −24.362 | 59.698 | −4.273 | 1.00 | 38.18 | C |
| ATOM | 6296 | O | CYS | C | 148 | −25.331 | 59.015 | −4.015 | 1.00 | 39.67 | O |
| ATOM | 6297 | N | ARG | C | 149 | −23.501 | 60.018 | −3.332 | 1.00 | 39.02 | N |
| ATOM | 6299 | CA | ARG | C | 149 | −23.731 | 59.551 | −1.986 | 1.00 | 42.05 | C |
| ATOM | 6301 | CB | ARG | C | 149 | −22.433 | 59.780 | −1.185 | 1.00 | 51.96 | C |
| ATOM | 6304 | CG | ARG | C | 149 | −22.509 | 59.724 | 0.324 | 1.00 | 54.52 | C |
| ATOM | 6307 | CD | ARG | C | 149 | −21.162 | 59.987 | 0.965 | 1.00 | 89.20 | C |
| ATOM | 6310 | NE | ARG | C | 149 | −20.948 | 61.404 | 1.270 | 1.00 | 106.43 | N |
| ATOM | 6312 | CZ | ARG | C | 149 | −19.877 | 62.091 | 0.879 | 1.00 | 119.92 | C |
| ATOM | 6313 | NH1 | ARG | C | 149 | −18.944 | 61.487 | 0.145 | 1.00 | 114.93 | N |
| ATOM | 6316 | NH2 | ARG | C | 149 | −19.740 | 63.377 | 1.209 | 1.00 | 117.14 | N |
| ATOM | 6319 | C | ARG | C | 149 | −24.927 | 60.338 | −1.470 | 1.00 | 39.38 | C |
| ATOM | 6320 | O | ARG | C | 149 | −25.924 | 59.752 | −1.046 | 1.00 | 39.80 | O |
| ATOM | 6321 | N | ASP | C | 150 | −24.844 | 61.667 | −1.561 | 1.00 | 35.81 | N |
| ATOM | 6323 | CA | ASP | C | 150 | −25.906 | 62.501 | −1.046 | 1.00 | 36.47 | C |
| ATOM | 6325 | CB | ASP | C | 150 | −25.591 | 63.949 | −1.325 | 1.00 | 46.50 | C |
| ATOM | 6328 | CG | ASP | C | 150 | −24.567 | 64.505 | −0.365 | 1.00 | 51.80 | C |
| ATOM | 6329 | OD1 | ASP | C | 150 | −24.201 | 63.767 | 0.577 | 1.00 | 52.49 | O |
| ATOM | 6330 | OD2 | ASP | C | 150 | −24.085 | 65.652 | −0.506 | 1.00 | 39.93 | O |
| ATOM | 6331 | C | ASP | C | 150 | −27.290 | 62.133 | −1.559 | 1.00 | 35.22 | C |
| ATOM | 6332 | O | ASP | C | 150 | −28.194 | 61.980 | −0.757 | 1.00 | 30.43 | O |
| ATOM | 6333 | N | LEU | C | 151 | −27.460 | 61.956 | −2.870 | 1.00 | 36.16 | N |
| ATOM | 6335 | CA | LEU | C | 151 | −28.795 | 61.786 | −3.442 | 1.00 | 38.59 | C |
| ATOM | 6337 | CB | LEU | C | 151 | −28.781 | 61.937 | −4.949 | 1.00 | 41.41 | C |
| ATOM | 6340 | CG | LEU | C | 151 | −28.493 | 63.306 | −5.546 | 1.00 | 44.18 | C |
| ATOM | 6342 | CD1 | LEU | C | 151 | −28.636 | 63.267 | −7.081 | 1.00 | 53.55 | C |
| ATOM | 6346 | CD2 | LEU | C | 151 | −29.450 | 64.235 | −4.927 | 1.00 | 38.78 | C |
| ATOM | 6350 | C | LEU | C | 151 | −29.374 | 60.416 | −3.133 | 1.00 | 45.29 | C |
| ATOM | 6351 | O | LEU | C | 151 | −30.585 | 60.193 | −3.149 | 1.00 | 54.57 | O |
| ATOM | 6352 | N | SER | C | 152 | −28.487 | 59.486 | −2.833 | 1.00 | 49.50 | N |
| ATOM | 6354 | CA | SER | C | 152 | −28.917 | 58.176 | −2.421 | 1.00 | 38.62 | C |
| ATOM | 6356 | CB | SER | C | 152 | −27.745 | 57.210 | −2.544 | 1.00 | 29.74 | C |
| ATOM | 6359 | OG | SER | C | 152 | −26.792 | 57.465 | −1.550 | 1.00 | 40.73 | O |
| ATOM | 6361 | C | SER | C | 152 | −29.417 | 58.269 | −0.994 | 1.00 | 41.44 | C |
| ATOM | 6362 | O | SER | C | 152 | −29.931 | 57.278 | −0.523 | 1.00 | 43.27 | O |
| ATOM | 6363 | N | HIS | C | 153 | −29.359 | 59.430 | −0.318 | 1.00 | 46.98 | N |
| ATOM | 6365 | CA | HIS | C | 153 | −30.235 | 59.652 | 0.862 | 1.00 | 46.17 | C |
| ATOM | 6367 | CB | HIS | C | 153 | −29.725 | 60.701 | 1.848 | 1.00 | 29.90 | C |
| ATOM | 6370 | CG | HIS | C | 153 | −28.367 | 60.388 | 2.385 | 1.00 | 51.52 | C |
| ATOM | 6371 | ND1 | HIS | C | 153 | −27.619 | 61.289 | 3.113 | 1.00 | 69.16 | N |
| ATOM | 6373 | CE1 | HIS | C | 153 | −26.434 | 60.763 | 3.359 | 1.00 | 81.33 | C |
| ATOM | 6375 | NE2 | HIS | C | 153 | −26.378 | 59.566 | 2.795 | 1.00 | 64.46 | N |
| ATOM | 6377 | CD2 | HIS | C | 153 | −27.585 | 59.295 | 2.210 | 1.00 | 57.51 | C |
| ATOM | 6379 | C | HIS | C | 153 | −31.718 | 59.872 | 0.557 | 1.00 | 49.01 | C |
| ATOM | 6380 | O | HIS | C | 153 | −32.595 | 59.641 | 1.403 | 1.00 | 52.31 | O |
| ATOM | 6381 | N | ILE | C | 154 | −31.995 | 60.210 | −0.693 | 1.00 | 49.95 | N |
| ATOM | 6383 | CA | ILE | C | 154 | −33.273 | 60.773 | −1.031 | 1.00 | 49.22 | C |
| ATOM | 6385 | CB | ILE | C | 154 | −33.087 | 61.999 | −1.918 | 1.00 | 47.89 | C |
| ATOM | 6387 | CG1 | ILE | C | 154 | −32.194 | 63.039 | −1.220 | 1.00 | 54.67 | C |
| ATOM | 6390 | CD1 | ILE | C | 154 | −32.727 | 63.557 | 0.117 | 1.00 | 45.84 | C |
| ATOM | 6394 | CG2 | ILE | C | 154 | −34.429 | 62.610 | −2.192 | 1.00 | 53.55 | C |
| ATOM | 6398 | C | ILE | C | 154 | −34.046 | 59.704 | −1.757 | 1.00 | 50.48 | C |
| ATOM | 6399 | O | ILE | C | 154 | −35.208 | 59.439 | −1.456 | 1.00 | 57.15 | O |
| ATOM | 6400 | N | GLY | C | 155 | −33.406 | 59.100 | −2.741 | 1.00 | 51.93 | N |
| ATOM | 6402 | CA | GLY | C | 155 | −34.121 | 58.264 | −3.684 | 1.00 | 48.62 | C |
| ATOM | 6405 | C | GLY | C | 155 | −33.092 | 57.277 | −4.142 | 1.00 | 46.72 | C |
| ATOM | 6406 | O | GLY | C | 155 | −31.947 | 57.321 | −3.697 | 1.00 | 52.83 | O |
| ATOM | 6407 | N | ASP | C | 156 | −33.470 | 56.412 | −5.062 | 1.00 | 50.89 | N |
| ATOM | 6409 | CA | ASP | C | 156 | −32.536 | 55.393 | −5.507 | 1.00 | 48.88 | C |
| ATOM | 6411 | CB | ASP | C | 156 | −32.966 | 54.076 | −4.875 | 1.00 | 46.60 | C |
| ATOM | 6414 | CG | ASP | C | 156 | −34.290 | 53.610 | −5.373 | 1.00 | 44.58 | C |
| ATOM | 6415 | OD1 | ASP | C | 156 | −34.556 | 53.847 | −6.578 | 1.00 | 43.35 | O |
| ATOM | 6416 | OD2 | ASP | C | 156 | −35.077 | 52.967 | −4.636 | 1.00 | 60.02 | O |
| ATOM | 6417 | C | ASP | C | 156 | −32.385 | 55.317 | −7.026 | 1.00 | 48.60 | C |
| ATOM | 6418 | O | ASP | C | 156 | −31.907 | 54.319 | −7.580 | 1.00 | 60.98 | O |
| ATOM | 6419 | N | ALA | C | 157 | −32.781 | 56.406 | −7.681 | 1.00 | 46.32 | N |
| ATOM | 6421 | CA | ALA | C | 157 | −32.440 | 56.675 | −9.066 | 1.00 | 39.86 | C |
| ATOM | 6423 | CB | ALA | C | 157 | −33.590 | 56.400 | −9.948 | 1.00 | 46.38 | C |
| ATOM | 6427 | C | ALA | C | 157 | −32.112 | 58.133 | −9.200 | 1.00 | 42.75 | C |
| ATOM | 6428 | O | ALA | C | 157 | −32.681 | 58.978 | −8.520 | 1.00 | 47.49 | O |
| ATOM | 6429 | N | VAL | C | 158 | −31.179 | 58.429 | −10.090 | 1.00 | 42.83 | N |
| ATOM | 6431 | CA | VAL | C | 158 | −30.824 | 59.801 | −10.315 | 1.00 | 42.20 | C |
| ATOM | 6433 | CB | VAL | C | 158 | −29.353 | 60.070 | −10.009 | 1.00 | 43.41 | C |
| ATOM | 6435 | CG1 | VAL | C | 158 | −28.471 | 59.152 | −10.773 | 1.00 | 36.64 | C |
| ATOM | 6439 | CG2 | VAL | C | 158 | −29.007 | 61.501 | −10.356 | 1.00 | 58.51 | C |
| ATOM | 6443 | C | VAL | C | 158 | −31.148 | 60.101 | −11.771 | 1.00 | 44.81 | C |
| ATOM | 6444 | O | VAL | C | 158 | −30.960 | 59.272 | −12.681 | 1.00 | 39.67 | O |
| ATOM | 6445 | N | VAL | C | 159 | −31.722 | 61.279 | −11.961 | 1.00 | 37.63 | N |
| ATOM | 6447 | CA | VAL | C | 159 | −31.898 | 61.790 | −13.289 | 1.00 | 42.15 | C |
| ATOM | 6449 | CB | VAL | C | 159 | −33.258 | 62.454 | −13.409 | 1.00 | 43.33 | C |
| ATOM | 6451 | CG1 | VAL | C | 159 | −33.504 | 62.935 | −14.843 | 1.00 | 41.59 | C |
| ATOM | 6455 | CG2 | VAL | C | 159 | −34.313 | 61.465 | −12.958 | 1.00 | 50.36 | C |
| ATOM | 6459 | C | VAL | C | 159 | −30.845 | 62.825 | −13.528 | 1.00 | 38.66 | C |
| ATOM | 6460 | O | VAL | C | 159 | −30.789 | 63.820 | −12.820 | 1.00 | 37.92 | O |
| ATOM | 6461 | N | ILE | C | 160 | −30.014 | 62.563 | −14.527 | 1.00 | 40.16 | N |
| ATOM | 6463 | CA | ILE | C | 160 | −28.840 | 63.376 | −14.792 | 1.00 | 40.21 | C |
| ATOM | 6465 | CB | ILE | C | 160 | −27.622 | 62.499 | −15.117 | 1.00 | 39.95 | C |
| ATOM | 6467 | CG1 | ILE | C | 160 | −27.356 | 61.466 | −14.026 | 1.00 | 35.87 | C |
| ATOM | 6470 | CD1 | ILE | C | 160 | −25.987 | 60.916 | −14.113 | 1.00 | 36.65 | C |
| ATOM | 6474 | CG2 | ILE | C | 160 | −26.384 | 63.339 | −15.324 | 1.00 | 31.96 | C |
| ATOM | 6478 | C | ILE | C | 160 | −29.307 | 63.974 | −16.073 | 1.00 | 45.90 | C |
| ATOM | 6479 | O | ILE | C | 160 | −29.706 | 63.235 | −16.971 | 1.00 | 57.20 | O |
| ATOM | 6480 | N | SER | C | 161 | −29.313 | 65.292 | −16.140 | 1.00 | 45.57 | N |
| ATOM | 6482 | CA | SER | C | 161 | −29.585 | 65.981 | −17.385 | 1.00 | 48.33 | C |
| ATOM | 6484 | CB | SER | C | 161 | −30.901 | 66.720 | −17.251 | 1.00 | 47.36 | C |
| ATOM | 6487 | OG | SER | C | 161 | −30.806 | 67.616 | −16.175 | 1.00 | 59.95 | O |
| ATOM | 6489 | C | SER | C | 161 | −28.487 | 66.999 | −17.634 | 1.00 | 50.11 | C |
| ATOM | 6490 | O | SER | C | 161 | −28.106 | 67.712 | −16.718 | 1.00 | 53.45 | O |
| ATOM | 6491 | N | CYS | C | 162 | −27.953 | 67.050 | −18.852 | 1.00 | 57.53 | N |
| ATOM | 6493 | CA | CYS | C | 162 | −26.786 | 67.885 | −19.157 | 1.00 | 58.22 | C |
| ATOM | 6495 | CB | CYS | C | 162 | −25.535 | 67.033 | −19.456 | 1.00 | 49.26 | C |
| ATOM | 6498 | SG | CYS | C | 162 | −24.286 | 67.642 | −20.618 | 1.00 | 88.60 | S |
| ATOM | 6499 | C | CYS | C | 162 | −27.179 | 68.860 | −20.263 | 1.00 | 48.82 | C |
| ATOM | 6500 | O | CYS | C | 162 | −28.065 | 68.605 | −21.056 | 1.00 | 48.78 | O |
| ATOM | 6501 | N | ALA | C | 163 | −26.577 | 70.034 | −20.224 | 1.00 | 50.57 | N |
| ATOM | 6503 | CA | ALA | C | 163 | −26.831 | 71.075 | −21.198 | 1.00 | 53.72 | C |
| ATOM | 6505 | CB | ALA | C | 163 | −28.027 | 71.842 | −20.747 | 1.00 | 54.44 | C |
| ATOM | 6509 | C | ALA | C | 163 | −25.611 | 72.007 | −21.369 | 1.00 | 58.50 | C |
| ATOM | 6510 | O | ALA | C | 163 | −24.609 | 71.892 | −20.666 | 1.00 | 62.31 | O |
| ATOM | 6511 | N | LYS | C | 164 | −25.682 | 72.954 | −22.297 | 1.00 | 61.85 | N |
| ATOM | 6513 | CA | LYS | C | 164 | −24.532 | 73.823 | −22.560 | 1.00 | 64.55 | C |
| ATOM | 6515 | CB | LYS | C | 164 | −24.934 | 74.944 | −23.537 | 1.00 | 60.72 | C |
| ATOM | 6518 | CG | LYS | C | 164 | −23.859 | 75.493 | −24.492 | 1.00 | 50.80 | C |
| ATOM | 6521 | CD | LYS | C | 164 | −24.423 | 75.878 | −25.908 | 1.00 | 77.38 | C |
| ATOM | 6524 | CE | LYS | C | 164 | −25.169 | 77.270 | −26.087 | 1.00 | 84.58 | C |
| ATOM | 6527 | NZ | LYS | C | 164 | −26.351 | 77.356 | −27.073 | 1.00 | 65.17 | N |
| ATOM | 6531 | C | LYS | C | 164 | −23.969 | 74.380 | −21.245 | 1.00 | 64.37 | C |
| ATOM | 6532 | O | LYS | C | 164 | −22.786 | 74.666 | −21.136 | 1.00 | 72.45 | O |
| ATOM | 6533 | N | ASP | C | 165 | −24.807 | 74.498 | −20.228 | 1.00 | 64.27 | N |
| ATOM | 6535 | CA | ASP | C | 165 | −24.577 | 75.466 | −19.164 | 1.00 | 69.59 | C |
| ATOM | 6537 | CB | ASP | C | 165 | −25.855 | 76.270 | −18.844 | 1.00 | 72.36 | C |
| ATOM | 6540 | CG | ASP | C | 165 | −27.146 | 75.489 | −19.112 | 1.00 | 83.47 | C |
| ATOM | 6541 | OD1 | ASP | C | 165 | −27.793 | 76.064 | −18.127 | 1.00 | 77.37 | O |
| ATOM | 6542 | OD2 | ASP | C | 165 | −27.628 | 75.315 | −20.264 | 1.00 | 86.04 | O |
| ATOM | 6543 | C | ASP | C | 165 | −24.094 | 74.778 | −17.889 | 1.00 | 65.64 | C |
| ATOM | 6544 | O | ASP | C | 165 | −23.471 | 75.395 | −17.021 | 1.00 | 64.29 | O |
| ATOM | 6545 | N | GLY | C | 166 | −24.432 | 73.505 | −17.751 | 1.00 | 57.99 | N |
| ATOM | 6547 | CA | GLY | C | 166 | −25.010 | 73.047 | −16.510 | 1.00 | 52.55 | C |
| ATOM | 6550 | C | GLY | C | 166 | −25.189 | 71.555 | −16.628 | 1.00 | 50.43 | C |
| ATOM | 6551 | O | GLY | C | 166 | −25.693 | 71.060 | −17.630 | 1.00 | 51.48 | O |
| ATOM | 6552 | N | VAL | C | 167 | −24.822 | 70.823 | −15.590 | 1.00 | 41.58 | N |
| ATOM | 6554 | CA | VAL | C | 167 | −25.331 | 69.480 | −15.485 | 1.00 | 39.49 | C |
| ATOM | 6556 | CB | VAL | C | 167 | −24.232 | 68.433 | −15.568 | 1.00 | 35.40 | C |
| ATOM | 6558 | CG1 | VAL | C | 167 | −23.199 | 68.786 | −14.562 | 1.00 | 29.77 | C |
| ATOM | 6562 | CG2 | VAL | C | 167 | −24.824 | 66.994 | −15.321 | 1.00 | 34.63 | C |
| ATOM | 6566 | C | VAL | C | 167 | −26.000 | 69.385 | −14.138 | 1.00 | 40.36 | C |
| ATOM | 6567 | O | VAL | C | 167 | −25.593 | 70.083 | −13.234 | 1.00 | 35.01 | O |
| ATOM | 6568 | N | LYS | C | 168 | −26.993 | 68.500 | −14.039 | 1.00 | 43.33 | N |
| ATOM | 6570 | CA | LYS | C | 168 | −28.026 | 68.517 | −13.016 | 1.00 | 39.77 | C |
| ATOM | 6572 | CB | LYS | C | 168 | −29.324 | 69.144 | −13.533 | 1.00 | 42.71 | C |
| ATOM | 6575 | CG | LYS | C | 168 | −30.590 | 68.697 | −12.763 | 1.00 | 43.88 | C |
| ATOM | 6578 | CD | LYS | C | 168 | −31.578 | 69.839 | −12.502 | 1.00 | 57.64 | C |
| ATOM | 6581 | CE | LYS | C | 168 | −32.686 | 69.948 | −13.575 | 1.00 | 67.44 | C |
| ATOM | 6584 | NZ | LYS | C | 168 | −33.884 | 70.684 | −13.069 | 1.00 | 67.48 | N |
| ATOM | 6588 | C | LYS | C | 168 | −28.337 | 67.092 | −12.558 | 1.00 | 39.69 | C |
| ATOM | 6589 | O | LYS | C | 168 | −28.719 | 66.226 | −13.358 | 1.00 | 39.14 | O |
| ATOM | 6590 | N | PHE | C | 169 | −28.162 | 66.865 | −11.258 | 1.00 | 32.66 | N |
| ATOM | 6592 | CA | PHE | C | 169 | −28.350 | 65.550 | −10.705 | 1.00 | 34.74 | C |
| ATOM | 6594 | CB | PHE | C | 169 | −27.118 | 65.081 | −9.936 | 1.00 | 37.70 | C |
| ATOM | 6597 | CG | PHE | C | 169 | −25.869 | 65.022 | −10.765 | 1.00 | 28.67 | C |
| ATOM | 6598 | CD1 | PHE | C | 169 | −25.083 | 66.150 | −10.927 | 1.00 | 44.45 | C |
| ATOM | 6600 | CE1 | PHE | C | 169 | −23.932 | 66.120 | −11.717 | 1.00 | 48.92 | C |
| ATOM | 6602 | CZ | PHE | C | 169 | −23.550 | 64.940 | −12.322 | 1.00 | 38.56 | C |
| ATOM | 6604 | CE2 | PHE | C | 169 | −24.318 | 63.795 | −12.161 | 1.00 | 47.51 | C |
| ATOM | 6606 | CD2 | PHE | C | 169 | −25.487 | 63.852 | −11.393 | 1.00 | 41.94 | C |
| ATOM | 6608 | C | PHE | C | 169 | −29.551 | 65.627 | −9.810 | 1.00 | 38.74 | C |
| ATOM | 6609 | O | PHE | C | 169 | −29.589 | 66.426 | −8.869 | 1.00 | 42.02 | O |
| ATOM | 6610 | N | SER | C | 170 | −30.536 | 64.782 | −10.090 | 1.00 | 40.34 | N |
| ATOM | 6612 | CA | SER | C | 170 | −31.790 | 64.899 | −9.366 | 1.00 | 43.74 | C |
| ATOM | 6614 | CB | SER | C | 170 | −32.718 | 65.844 | −10.146 | 1.00 | 39.49 | C |
| ATOM | 6617 | OG | SER | C | 170 | −33.793 | 65.110 | −10.674 | 1.00 | 57.91 | O |
| ATOM | 6619 | C | SER | C | 170 | −32.428 | 63.544 | −9.001 | 1.00 | 37.42 | C |
| ATOM | 6620 | O | SER | C | 170 | −32.348 | 62.583 | −9.756 | 1.00 | 41.05 | O |
| ATOM | 6621 | N | ALA | C | 171 | −32.948 | 63.434 | −7.781 | 1.00 | 40.73 | N |
| ATOM | 6623 | CA | ALA | C | 171 | −33.582 | 62.206 | −7.311 | 1.00 | 35.85 | C |
| ATOM | 6625 | CB | ALA | C | 171 | −32.626 | 61.381 | −6.448 | 1.00 | 44.99 | C |
| ATOM | 6629 | C | ALA | C | 171 | −34.810 | 62.500 | −6.486 | 1.00 | 36.85 | C |
| ATOM | 6630 | O | ALA | C | 171 | −34.989 | 63.579 | −5.958 | 1.00 | 38.40 | O |
| ATOM | 6631 | N | SER | C | 172 | −35.604 | 61.460 | −6.288 | 1.00 | 46.51 | N |
| ATOM | 6633 | CA | SER | C | 172 | −36.803 | 61.603 | −5.505 | 1.00 | 45.22 | C |
| ATOM | 6635 | CB | SER | C | 172 | −37.971 | 61.906 | −6.411 | 1.00 | 44.91 | C |
| ATOM | 6638 | OG | SER | C | 172 | −38.991 | 62.410 | −5.562 | 1.00 | 74.28 | O |
| ATOM | 6640 | C | SER | C | 172 | −37.190 | 60.376 | −4.734 | 1.00 | 33.95 | C |
| ATOM | 6641 | O | SER | C | 172 | −37.013 | 59.290 | −5.206 | 1.00 | 37.84 | O |
| ATOM | 6642 | N | GLY | C | 173 | −37.839 | 60.579 | −3.600 | 1.00 | 39.49 | N |
| ATOM | 6644 | CA | GLY | C | 173 | −38.274 | 59.488 | −2.762 | 1.00 | 43.18 | C |
| ATOM | 6647 | C | GLY | C | 173 | −39.429 | 59.780 | −1.825 | 1.00 | 47.07 | C |
| ATOM | 6648 | O | GLY | C | 173 | −40.116 | 60.783 | −1.945 | 1.00 | 39.81 | O |
| ATOM | 6649 | N | GLU | C | 174 | −39.624 | 58.892 | −0.855 | 1.00 | 55.67 | N |
| ATOM | 6651 | CA | GLU | C | 174 | −40.654 | 59.098 | 0.148 | 1.00 | 60.93 | C |
| ATOM | 6653 | CB | GLU | C | 174 | −40.617 | 57.995 | 1.199 | 1.00 | 65.57 | C |
| ATOM | 6656 | CG | GLU | C | 174 | −41.975 | 57.754 | 1.835 | 1.00 | 84.15 | C |
| ATOM | 6659 | CD | GLU | C | 174 | −42.620 | 56.476 | 1.326 | 1.00 | 102.22 | C |
| ATOM | 6660 | OE1 | GLU | C | 174 | −43.114 | 56.449 | 0.172 | 1.00 | 86.97 | O |
| ATOM | 6661 | OE2 | GLU | C | 174 | −42.610 | 55.485 | 2.090 | 1.00 | 124.94 | O |
| ATOM | 6662 | C | GLU | C | 174 | −40.576 | 60.456 | 0.836 | 1.00 | 61.27 | C |
| ATOM | 6663 | O | GLU | C | 174 | −41.617 | 61.093 | 1.032 | 1.00 | 65.33 | O |
| ATOM | 6664 | N | LEU | C | 175 | −39.377 | 60.893 | 1.226 | 1.00 | 54.85 | N |
| ATOM | 6666 | CA | LEU | C | 175 | −39.298 | 62.002 | 2.162 | 1.00 | 54.59 | C |
| ATOM | 6668 | CB | LEU | C | 175 | −38.039 | 61.963 | 3.033 | 1.00 | 56.93 | C |
| ATOM | 6671 | CG | LEU | C | 175 | −36.714 | 61.834 | 2.291 | 1.00 | 65.08 | C |
| ATOM | 6673 | CD1 | LEU | C | 175 | −36.417 | 63.147 | 1.649 | 1.00 | 68.66 | C |
| ATOM | 6677 | CD2 | LEU | C | 175 | −35.564 | 61.423 | 3.215 | 1.00 | 62.24 | C |
| ATOM | 6681 | C | LEU | C | 175 | −39.430 | 63.287 | 1.383 | 1.00 | 47.28 | C |
| ATOM | 6682 | O | LEU | C | 175 | −39.814 | 64.326 | 1.896 | 1.00 | 54.89 | O |
| ATOM | 6683 | N | GLY | C | 176 | −39.217 | 63.190 | 0.090 | 1.00 | 48.58 | N |
| ATOM | 6685 | CA | GLY | C | 176 | −39.302 | 64.372 | −0.737 | 1.00 | 44.45 | C |
| ATOM | 6688 | C | GLY | C | 176 | −38.386 | 64.240 | −1.936 | 1.00 | 44.41 | C |
| ATOM | 6689 | O | GLY | C | 176 | −38.199 | 63.156 | −2.515 | 1.00 | 42.56 | O |
| ATOM | 6690 | N | ASN | C | 177 | −37.817 | 65.358 | −2.344 | 1.00 | 31.16 | N |
| ATOM | 6692 | CA | ASN | C | 177 | −36.974 | 65.261 | −3.506 | 1.00 | 42.39 | C |
| ATOM | 6694 | CB | ASN | C | 177 | −37.780 | 65.187 | −4.789 | 1.00 | 43.55 | C |
| ATOM | 6697 | CG | ASN | C | 177 | −38.630 | 66.412 | −5.012 | 1.00 | 58.67 | C |
| ATOM | 6698 | OD1 | ASN | C | 177 | −38.145 | 67.572 | −5.122 | 1.00 | 43.36 | O |
| ATOM | 6699 | ND2 | ASN | C | 177 | −39.936 | 66.162 | −5.041 | 1.00 | 62.13 | N |
| ATOM | 6702 | C | ASN | C | 177 | −35.983 | 66.376 | −3.571 | 1.00 | 41.12 | C |
| ATOM | 6703 | O | ASN | C | 177 | −35.894 | 67.203 | −2.656 | 1.00 | 45.65 | O |
| ATOM | 6704 | N | GLY | C | 178 | −35.165 | 66.312 | −4.610 | 1.00 | 39.99 | N |
| ATOM | 6706 | CA | GLY | C | 178 | −33.812 | 66.793 | −4.464 | 1.00 | 44.93 | C |
| ATOM | 6709 | C | GLY | C | 178 | −33.041 | 66.923 | −5.755 | 1.00 | 40.17 | C |
| ATOM | 6710 | O | GLY | C | 178 | −33.078 | 66.063 | −6.631 | 1.00 | 30.15 | O |
| ATOM | 6711 | N | ASN | C | 179 | −32.269 | 67.989 | −5.843 | 1.00 | 36.58 | N |
| ATOM | 6713 | CA | ASN | C | 179 | −31.979 | 68.490 | −7.156 | 1.00 | 45.39 | C |
| ATOM | 6715 | CB | ASN | C | 179 | −33.166 | 69.377 | −7.506 | 1.00 | 55.39 | C |
| ATOM | 6718 | CG | ASN | C | 179 | −33.194 | 69.725 | −8.930 | 1.00 | 72.41 | C |
| ATOM | 6719 | OD1 | ASN | C | 179 | −33.762 | 68.989 | −9.748 | 1.00 | 101.83 | O |
| ATOM | 6720 | ND2 | ASN | C | 179 | −32.525 | 70.822 | −9.265 | 1.00 | 90.35 | N |
| ATOM | 6723 | C | ASN | C | 179 | −30.678 | 69.251 | −7.092 | 1.00 | 36.53 | C |
| ATOM | 6724 | O | ASN | C | 179 | −30.603 | 70.297 | −6.458 | 1.00 | 44.23 | O |
| ATOM | 6725 | N | ILE | C | 180 | −29.611 | 68.665 | −7.609 | 1.00 | 36.44 | N |
| ATOM | 6727 | CA | ILE | C | 180 | −28.295 | 69.291 | −7.479 | 1.00 | 34.02 | C |
| ATOM | 6729 | CB | ILE | C | 180 | −27.280 | 68.284 | −7.020 | 1.00 | 33.25 | C |
| ATOM | 6731 | CG1 | ILE | C | 180 | −27.604 | 67.850 | −5.593 | 1.00 | 33.05 | C |
| ATOM | 6734 | CD1 | ILE | C | 180 | −26.990 | 66.558 | −5.222 | 1.00 | 38.83 | C |
| ATOM | 6738 | CG2 | ILE | C | 180 | −25.889 | 68.908 | −7.073 | 1.00 | 42.00 | C |
| ATOM | 6742 | C | ILE | C | 180 | −27.801 | 69.823 | −8.801 | 1.00 | 37.21 | C |
| ATOM | 6743 | O | ILE | C | 180 | −27.733 | 69.064 | −9.756 | 1.00 | 41.42 | O |
| ATOM | 6744 | N | LYS | C | 181 | −27.398 | 71.090 | −8.851 | 1.00 | 38.76 | N |
| ATOM | 6746 | CA | LYS | C | 181 | −26.958 | 71.672 | −10.111 | 1.00 | 41.27 | C |
| ATOM | 6748 | CB | LYS | C | 181 | −27.910 | 72.761 | −10.641 | 1.00 | 36.76 | C |
| ATOM | 6751 | CG | LYS | C | 181 | −27.924 | 72.838 | −12.204 | 1.00 | 67.66 | C |
| ATOM | 6754 | CD | LYS | C | 181 | −28.881 | 73.882 | −12.849 | 1.00 | 106.69 | C |
| ATOM | 6757 | CE | LYS | C | 181 | −30.347 | 73.794 | −12.337 | 1.00 | 124.18 | C |
| ATOM | 6760 | NZ | LYS | C | 181 | −31.421 | 74.271 | −13.287 | 1.00 | 124.38 | N |
| ATOM | 6764 | C | LYS | C | 181 | −25.504 | 72.144 | −10.129 | 1.00 | 38.99 | C |
| ATOM | 6765 | O | LYS | C | 181 | −25.133 | 73.083 | −9.440 | 1.00 | 38.73 | O |
| ATOM | 6766 | N | LEU | C | 182 | −24.718 | 71.574 | −11.036 | 1.00 | 41.21 | N |
| ATOM | 6768 | CA | LEU | C | 182 | −23.393 | 72.099 | −11.351 | 1.00 | 47.67 | C |
| ATOM | 6770 | CB | LEU | C | 182 | −22.435 | 70.920 | −11.481 | 1.00 | 50.48 | C |
| ATOM | 6773 | CG | LEU | C | 182 | −22.261 | 70.152 | −10.178 | 1.00 | 40.69 | C |
| ATOM | 6775 | CD1 | LEU | C | 182 | −21.209 | 69.122 | −10.385 | 1.00 | 56.01 | C |
| ATOM | 6779 | CD2 | LEU | C | 182 | −21.844 | 71.134 | −9.117 | 1.00 | 36.77 | C |
| ATOM | 6783 | C | LEU | C | 182 | −23.331 | 72.930 | −12.631 | 1.00 | 50.72 | C |
| ATOM | 6784 | O | LEU | C | 182 | −23.710 | 72.477 | −13.712 | 1.00 | 50.66 | O |
| ATOM | 6785 | N | SER | C | 183 | −22.803 | 74.139 | −12.493 | 1.00 | 51.57 | N |
| ATOM | 6787 | CA | SER | C | 183 | −22.650 | 75.051 | −13.612 | 1.00 | 49.52 | C |
| ATOM | 6789 | CB | SER | C | 183 | −22.760 | 76.472 | −13.111 | 1.00 | 53.57 | C |
| ATOM | 6792 | OG | SER | C | 183 | −23.976 | 76.593 | −12.401 | 1.00 | 70.25 | O |
| ATOM | 6794 | C | SER | C | 183 | −21.310 | 74.909 | −14.259 | 1.00 | 54.33 | C |
| ATOM | 6795 | O | SER | C | 183 | −20.311 | 74.651 | −13.580 | 1.00 | 56.31 | O |
| ATOM | 6796 | N | GLN | C | 184 | −21.289 | 75.029 | −15.582 | 1.00 | 64.33 | N |
| ATOM | 6798 | CA | GLN | C | 184 | −20.043 | 74.773 | −16.300 | 1.00 | 71.79 | C |
| ATOM | 6800 | CB | GLN | C | 184 | −20.229 | 74.602 | −17.817 | 1.00 | 73.38 | C |
| ATOM | 6803 | CG | GLN | C | 184 | −18.963 | 74.842 | −18.672 | 1.00 | 69.80 | C |
| ATOM | 6806 | CD | GLN | C | 184 | −19.107 | 74.325 | −20.094 | 1.00 | 66.93 | C |
| ATOM | 6807 | OE1 | GLN | C | 184 | −20.224 | 74.229 | −20.598 | 1.00 | 81.57 | O |
| ATOM | 6808 | NE2 | GLN | C | 184 | −17.989 | 73.984 | −20.743 | 1.00 | 56.80 | N |
| ATOM | 6811 | C | GLN | C | 184 | −19.245 | 76.010 | −15.964 | 1.00 | 79.07 | C |
| ATOM | 6812 | O | GLN | C | 184 | −19.716 | 77.133 | −16.189 | 1.00 | 82.23 | O |
| ATOM | 6813 | N | THR | C | 185 | −18.123 | 75.791 | −15.289 | 1.00 | 82.58 | N |
| ATOM | 6815 | CA | THR | C | 185 | −17.457 | 76.850 | −14.559 | 1.00 | 88.69 | C |
| ATOM | 6817 | CB | THR | C | 185 | −15.974 | 76.694 | −14.843 | 1.00 | 88.18 | C |
| ATOM | 6819 | OG1 | THR | C | 185 | −15.802 | 76.512 | −16.254 | 1.00 | 74.29 | O |
| ATOM | 6821 | CG2 | THR | C | 185 | −15.489 | 75.366 | −14.245 | 1.00 | 81.12 | C |
| ATOM | 6825 | C | THR | C | 185 | −18.020 | 78.218 | −14.999 | 1.00 | 100.02 | C |
| ATOM | 6826 | O | THR | C | 185 | −19.251 | 78.418 | −15.018 | 1.00 | 101.52 | O |
| ATOM | 6827 | N | SER | C | 186 | −17.129 | 79.149 | −15.352 | 1.00 | 104.72 | N |
| ATOM | 6829 | CA | SER | C | 186 | −17.492 | 80.525 | −15.713 | 1.00 | 101.97 | C |
| ATOM | 6831 | CB | SER | C | 186 | −18.329 | 81.214 | −14.611 | 1.00 | 108.88 | C |
| ATOM | 6834 | OG | SER | C | 186 | −19.716 | 81.393 | −14.913 | 1.00 | 101.75 | O |
| ATOM | 6836 | C | SER | C | 186 | −16.146 | 81.222 | −15.833 | 1.00 | 98.56 | C |
| ATOM | 6837 | O | SER | C | 186 | −15.740 | 81.609 | −16.904 | 1.00 | 80.16 | O |
| ATOM | 6838 | N | ASN | C | 187 | −15.421 | 81.302 | −14.724 | 1.00 | 109.58 | N |
| ATOM | 6840 | CA | ASN | C | 187 | −14.070 | 81.863 | −14.727 | 1.00 | 118.17 | C |
| ATOM | 6842 | CB | ASN | C | 187 | −13.621 | 82.239 | −13.298 | 1.00 | 114.06 | C |
| ATOM | 6845 | CG | ASN | C | 187 | −14.516 | 81.656 | −12.207 | 1.00 | 109.31 | C |
| ATOM | 6846 | OD1 | ASN | C | 187 | −14.195 | 80.620 | −11.618 | 1.00 | 111.33 | O |
| ATOM | 6847 | ND2 | ASN | C | 187 | −15.623 | 82.337 | −11.910 | 1.00 | 95.87 | N |
| ATOM | 6850 | C | ASN | C | 187 | −13.048 | 80.915 | −15.387 | 1.00 | 130.60 | C |
| ATOM | 6851 | O | ASN | C | 187 | −13.415 | 79.969 | −16.106 | 1.00 | 131.97 | O |
| ATOM | 6852 | N | VAL | C | 188 | −11.765 | 81.172 | −15.130 | 1.00 | 137.19 | N |
| ATOM | 6854 | CA | VAL | C | 188 | −10.673 | 80.389 | −15.709 | 1.00 | 140.07 | C |
| ATOM | 6856 | CB | VAL | C | 188 | −10.323 | 80.968 | −17.118 | 1.00 | 139.17 | C |
| ATOM | 6858 | CG1 | VAL | C | 188 | −8.817 | 81.063 | −17.365 | 1.00 | 137.00 | C |
| ATOM | 6862 | CG2 | VAL | C | 188 | −11.029 | 80.170 | −18.224 | 1.00 | 131.11 | C |
| ATOM | 6866 | C | VAL | C | 188 | −9.473 | 80.333 | −14.722 | 1.00 | 147.51 | C |
| ATOM | 6867 | O | VAL | C | 188 | −8.843 | 81.364 | −14.452 | 1.00 | 147.94 | O |
| ATOM | 6868 | N | ASP | C | 189 | −9.206 | 79.142 | −14.154 | 1.00 | 153.76 | N |
| ATOM | 6870 | CA | ASP | C | 189 | −8.327 | 78.909 | −12.972 | 1.00 | 151.37 | C |
| ATOM | 6872 | CB | ASP | C | 189 | −9.130 | 79.049 | −11.654 | 1.00 | 150.67 | C |
| ATOM | 6875 | CG | ASP | C | 189 | −8.668 | 78.087 | −10.549 | 1.00 | 146.71 | C |
| ATOM | 6876 | OD1 | ASP | C | 189 | −7.468 | 78.094 | −10.198 | 1.00 | 138.71 | O |
| ATOM | 6877 | OD2 | ASP | C | 189 | −9.443 | 77.314 | −9.935 | 1.00 | 137.11 | O |
| ATOM | 6878 | C | ASP | C | 189 | −7.597 | 77.535 | −13.002 | 1.00 | 147.96 | C |
| ATOM | 6879 | O | ASP | C | 189 | −6.413 | 77.438 | −12.641 | 1.00 | 143.73 | O |
| ATOM | 6880 | N | LYS | C | 190 | −8.342 | 76.481 | −13.362 | 1.00 | 142.95 | N |
| ATOM | 6882 | CA | LYS | C | 190 | −7.855 | 75.308 | −14.106 | 1.00 | 137.50 | C |
| ATOM | 6884 | CB | LYS | C | 190 | −6.318 | 75.272 | −14.216 | 1.00 | 137.17 | C |
| ATOM | 6887 | CG | LYS | C | 190 | −5.776 | 75.242 | −15.651 | 1.00 | 129.12 | C |
| ATOM | 6890 | CD | LYS | C | 190 | −4.611 | 76.217 | −15.826 | 1.00 | 124.67 | C |
| ATOM | 6893 | CE | LYS | C | 190 | −4.944 | 77.638 | −15.334 | 1.00 | 114.44 | C |
| ATOM | 6896 | NZ | LYS | C | 190 | −4.592 | 77.948 | −13.907 | 1.00 | 92.99 | N |
| ATOM | 6900 | C | LYS | C | 190 | −8.425 | 74.026 | −13.470 | 1.00 | 133.29 | C |
| ATOM | 6901 | O | LYS | C | 190 | −9.638 | 73.911 | −13.304 | 1.00 | 129.71 | O |
| ATOM | 6902 | N | GLU | C | 191 | −7.567 | 73.067 | −13.124 | 1.00 | 127.51 | N |
| ATOM | 6904 | CA | GLU | C | 191 | −7.714 | 72.305 | −11.887 | 1.00 | 121.70 | C |
| ATOM | 6906 | CB | GLU | C | 191 | −8.122 | 73.246 | −10.743 | 1.00 | 121.62 | C |
| ATOM | 6909 | CG | GLU | C | 191 | −6.957 | 73.948 | −10.037 | 1.00 | 111.24 | C |
| ATOM | 6912 | CD | GLU | C | 191 | −7.310 | 74.305 | −8.605 | 1.00 | 104.29 | C |
| ATOM | 6913 | OE1 | GLU | C | 191 | −6.784 | 73.655 | −7.679 | 1.00 | 108.66 | O |
| ATOM | 6914 | OE2 | GLU | C | 191 | −8.159 | 75.201 | −8.397 | 1.00 | 102.66 | O |
| ATOM | 6915 | C | GLU | C | 191 | −8.702 | 71.138 | −12.058 | 1.00 | 117.28 | C |
| ATOM | 6916 | O | GLU | C | 191 | −9.128 | 70.850 | −13.172 | 1.00 | 118.27 | O |
| ATOM | 6917 | N | GLU | C | 192 | −8.939 | 70.382 | −10.989 | 1.00 | 108.93 | N |
| ATOM | 6919 | CA | GLU | C | 192 | −10.261 | 70.191 | −10.397 | 1.00 | 102.77 | C |
| ATOM | 6921 | CB | GLU | C | 192 | −10.057 | 69.659 | −8.989 | 1.00 | 105.12 | C |
| ATOM | 6924 | CG | GLU | C | 192 | −10.239 | 68.168 | −8.783 | 1.00 | 109.55 | C |
| ATOM | 6927 | CD | GLU | C | 192 | −10.322 | 67.856 | −7.294 | 1.00 | 120.68 | C |
| ATOM | 6928 | OE1 | GLU | C | 192 | −11.292 | 67.178 | −6.851 | 1.00 | 109.18 | O |
| ATOM | 6929 | OE2 | GLU | C | 192 | −9.420 | 68.342 | −6.567 | 1.00 | 114.43 | O |
| ATOM | 6930 | C | GLU | C | 192 | −11.056 | 71.478 | −10.189 | 1.00 | 95.42 | C |
| ATOM | 6931 | O | GLU | C | 192 | −10.759 | 72.514 | −10.773 | 1.00 | 90.50 | O |
| ATOM | 6932 | N | GLU | C | 193 | −11.946 | 71.413 | −9.199 | 1.00 | 90.88 | N |
| ATOM | 6934 | CA | GLU | C | 193 | −13.254 | 72.087 | −9.153 | 1.00 | 86.96 | C |
| ATOM | 6936 | CB | GLU | C | 193 | −13.249 | 73.276 | −8.190 | 1.00 | 88.74 | C |
| ATOM | 6939 | CG | GLU | C | 193 | −13.642 | 72.910 | −6.757 | 1.00 | 94.81 | C |
| ATOM | 6942 | CD | GLU | C | 193 | −13.434 | 74.046 | −5.761 | 1.00 | 97.62 | C |
| ATOM | 6943 | OE1 | GLU | C | 193 | −12.721 | 73.833 | −4.763 | 1.00 | 98.65 | O |
| ATOM | 6944 | OE2 | GLU | C | 193 | −13.994 | 75.149 | −5.950 | 1.00 | 98.07 | O |
| ATOM | 6945 | C | GLU | C | 193 | −13.898 | 72.473 | −10.474 | 1.00 | 79.01 | C |
| ATOM | 6946 | O | GLU | C | 193 | −15.095 | 72.748 | −10.527 | 1.00 | 76.89 | O |
| ATOM | 6947 | N | ALA | C | 194 | −13.132 | 72.379 | −11.553 | 1.00 | 74.69 | N |
| ATOM | 6949 | CA | ALA | C | 194 | −13.619 | 72.739 | −12.883 | 1.00 | 69.85 | C |
| ATOM | 6951 | CB | ALA | C | 194 | −12.467 | 72.757 | −13.870 | 1.00 | 65.10 | C |
| ATOM | 6955 | C | ALA | C | 194 | −14.689 | 71.788 | −13.380 | 1.00 | 61.19 | C |
| ATOM | 6956 | O | ALA | C | 194 | −14.405 | 70.626 | −13.668 | 1.00 | 62.01 | O |
| ATOM | 6957 | N | VAL | C | 195 | −15.887 | 72.311 | −13.596 | 1.00 | 52.47 | N |
| ATOM | 6959 | CA | VAL | C | 195 | −16.821 | 71.591 | −14.439 | 1.00 | 47.29 | C |
| ATOM | 6961 | CB | VAL | C | 195 | −18.240 | 71.667 | −13.914 | 1.00 | 47.46 | C |
| ATOM | 6963 | CG1 | VAL | C | 195 | −19.188 | 70.991 | −14.906 | 1.00 | 48.55 | C |
| ATOM | 6967 | CG2 | VAL | C | 195 | −18.294 | 70.976 | −12.567 | 1.00 | 53.12 | C |
| ATOM | 6971 | C | VAL | C | 195 | −16.805 | 72.075 | −15.866 | 1.00 | 39.63 | C |
| ATOM | 6972 | O | VAL | C | 195 | −16.919 | 73.257 | −16.145 | 1.00 | 52.11 | O |
| ATOM | 6973 | N | THR | C | 196 | −16.630 | 71.149 | −16.786 | 1.00 | 39.68 | N |
| ATOM | 6975 | CA | THR | C | 196 | −16.628 | 71.511 | −18.189 | 1.00 | 39.80 | C |
| ATOM | 6977 | CB | THR | C | 196 | −15.213 | 71.575 | −18.730 | 1.00 | 27.33 | C |
| ATOM | 6979 | OG1 | THR | C | 196 | −15.156 | 70.738 | −19.891 | 1.00 | 46.46 | O |
| ATOM | 6981 | CG2 | THR | C | 196 | −14.280 | 70.838 | −17.769 | 1.00 | 54.13 | C |
| ATOM | 6985 | C | THR | C | 196 | −17.432 | 70.477 | −18.979 | 1.00 | 36.23 | C |
| ATOM | 6986 | O | THR | C | 196 | −17.374 | 69.277 | −18.711 | 1.00 | 32.56 | O |
| ATOM | 6987 | N | ILE | C | 197 | −18.129 | 70.968 | −19.998 | 1.00 | 34.88 | N |
| ATOM | 6989 | CA | ILE | C | 197 | −19.111 | 70.183 | −20.724 | 1.00 | 39.03 | C |
| ATOM | 6991 | CB | ILE | C | 197 | −20.497 | 70.736 | −20.339 | 1.00 | 32.64 | C |
| ATOM | 6993 | CG1 | ILE | C | 197 | −20.705 | 70.580 | −18.828 | 1.00 | 18.57 | C |
| ATOM | 6996 | CD1 | ILE | C | 197 | −22.020 | 71.098 | −18.297 | 1.00 | 44.60 | C |
| ATOM | 7000 | CG2 | ILE | C | 197 | −21.579 | 70.093 | −21.221 | 1.00 | 37.32 | C |
| ATOM | 7004 | C | ILE | C | 197 | −18.903 | 70.274 | −22.244 | 1.00 | 40.24 | C |
| ATOM | 7005 | O | ILE | C | 197 | −18.966 | 71.359 | −22.794 | 1.00 | 41.33 | O |
| ATOM | 7006 | N | GLU | C | 198 | −18.715 | 69.147 | −22.926 | 1.00 | 48.16 | N |
| ATOM | 7008 | CA | GLU | C | 198 | −18.737 | 69.130 | −24.388 | 1.00 | 52.76 | C |
| ATOM | 7010 | CB | GLU | C | 198 | −17.639 | 68.289 | −24.990 | 1.00 | 52.93 | C |
| ATOM | 7013 | CG | GLU | C | 198 | −16.350 | 69.081 | −25.127 | 1.00 | 85.64 | C |
| ATOM | 7016 | CD | GLU | C | 198 | −15.200 | 68.173 | −25.496 | 1.00 | 110.77 | C |
| ATOM | 7017 | OE1 | GLU | C | 198 | −14.850 | 68.144 | −26.700 | 1.00 | 120.46 | O |
| ATOM | 7018 | OE2 | GLU | C | 198 | −14.706 | 67.454 | −24.593 | 1.00 | 120.86 | O |
| ATOM | 7019 | C | GLU | C | 198 | −20.006 | 68.582 | −24.929 | 1.00 | 55.02 | C |
| ATOM | 7020 | O | GLU | C | 198 | −20.219 | 67.383 | −25.014 | 1.00 | 64.77 | O |
| ATOM | 7021 | N | MET | C | 199 | −20.818 | 69.501 | −25.394 | 1.00 | 61.71 | N |
| ATOM | 7023 | CA | MET | C | 199 | −22.205 | 69.210 | −25.612 | 1.00 | 62.85 | C |
| ATOM | 7025 | CB | MET | C | 199 | −22.988 | 70.307 | −24.916 | 1.00 | 72.48 | C |
| ATOM | 7028 | CG | MET | C | 199 | −24.364 | 69.881 | −24.531 | 1.00 | 77.29 | C |
| ATOM | 7031 | SD | MET | C | 199 | −25.409 | 70.923 | −25.471 | 1.00 | 67.93 | S |
| ATOM | 7032 | CE | MET | C | 199 | −26.965 | 70.383 | −24.915 | 1.00 | 67.69 | C |
| ATOM | 7036 | C | MET | C | 199 | −22.442 | 69.256 | −27.112 | 1.00 | 57.39 | C |
| ATOM | 7037 | O | MET | C | 199 | −21.879 | 70.071 | −27.822 | 1.00 | 63.63 | O |
| ATOM | 7038 | N | ASN | C | 200 | −23.217 | 68.315 | −27.610 | 1.00 | 53.36 | N |
| ATOM | 7040 | CA | ASN | C | 200 | −23.628 | 68.312 | −29.004 | 1.00 | 46.88 | C |
| ATOM | 7042 | CB | ASN | C | 200 | −23.323 | 66.945 | −29.598 | 1.00 | 41.47 | C |
| ATOM | 7045 | CG | ASN | C | 200 | −21.859 | 66.767 | −29.976 | 1.00 | 67.92 | C |
| ATOM | 7046 | OD1 | ASN | C | 200 | −21.010 | 67.550 | −29.541 | 1.00 | 99.99 | O |
| ATOM | 7047 | ND2 | ASN | C | 200 | −21.553 | 65.734 | −30.784 | 1.00 | 43.37 | N |
| ATOM | 7050 | C | ASN | C | 200 | −25.143 | 68.533 | −29.067 | 1.00 | 45.22 | C |
| ATOM | 7051 | O | ASN | C | 200 | −25.609 | 69.296 | −29.887 | 1.00 | 50.62 | O |
| ATOM | 7052 | N | GLU | C | 201 | −25.896 | 67.825 | −28.230 | 1.00 | 42.90 | N |
| ATOM | 7054 | CA | GLU | C | 201 | −27.236 | 68.180 | −27.814 | 1.00 | 47.82 | C |
| ATOM | 7056 | CB | GLU | C | 201 | −28.277 | 67.365 | −28.592 | 1.00 | 57.47 | C |
| ATOM | 7059 | CG | GLU | C | 201 | −27.982 | 67.172 | −30.068 | 1.00 | 63.60 | C |
| ATOM | 7062 | CD | GLU | C | 201 | −26.907 | 66.123 | −30.314 | 1.00 | 84.52 | C |
| ATOM | 7063 | OE1 | GLU | C | 201 | −26.827 | 65.116 | −29.576 | 1.00 | 86.62 | O |
| ATOM | 7064 | OE2 | GLU | C | 201 | −26.125 | 66.302 | −31.270 | 1.00 | 110.01 | O |
| ATOM | 7065 | C | GLU | C | 201 | −27.375 | 67.842 | −26.333 | 1.00 | 50.10 | C |
| ATOM | 7066 | O | GLU | C | 201 | −26.504 | 67.207 | −25.726 | 1.00 | 53.01 | O |
| ATOM | 7067 | N | PRO | C | 202 | −28.485 | 68.276 | −25.751 | 1.00 | 46.64 | N |
| ATOM | 7068 | CA | PRO | C | 202 | −28.773 | 68.023 | −24.349 | 1.00 | 46.35 | C |
| ATOM | 7070 | CB | PRO | C | 202 | −29.902 | 69.006 | −24.023 | 1.00 | 39.51 | C |
| ATOM | 7073 | CG | PRO | C | 202 | −30.069 | 69.808 | −25.158 | 1.00 | 41.64 | C |
| ATOM | 7076 | CD | PRO | C | 202 | −29.535 | 69.097 | −26.353 | 1.00 | 49.88 | C |
| ATOM | 7079 | C | PRO | C | 202 | −29.276 | 66.625 | −24.230 | 1.00 | 43.11 | C |
| ATOM | 7080 | O | PRO | C | 202 | −29.865 | 66.173 | −25.210 | 1.00 | 50.42 | O |
| ATOM | 7081 | N | VAL | C | 203 | −29.113 | 66.037 | −23.041 | 1.00 | 46.60 | N |
| ATOM | 7083 | CA | VAL | C | 203 | −29.507 | 64.665 | −22.766 | 1.00 | 48.91 | C |
| ATOM | 7085 | CB | VAL | C | 203 | −28.340 | 63.671 | −22.733 | 1.00 | 44.53 | C |
| ATOM | 7087 | CG1 | VAL | C | 203 | −28.780 | 62.378 | −23.312 | 1.00 | 57.87 | C |
| ATOM | 7091 | CG2 | VAL | C | 203 | −27.159 | 64.162 | −23.504 | 1.00 | 58.44 | C |
| ATOM | 7095 | C | VAL | C | 203 | −30.037 | 64.602 | −21.374 | 1.00 | 53.92 | C |
| ATOM | 7096 | O | VAL | C | 203 | −29.626 | 65.345 | −20.498 | 1.00 | 50.18 | O |
| ATOM | 7097 | N | GLN | C | 204 | −30.899 | 63.626 | −21.161 | 1.00 | 60.37 | N |
| ATOM | 7099 | CA | GLN | C | 204 | −31.427 | 63.411 | −19.839 | 1.00 | 56.80 | C |
| ATOM | 7101 | CB | GLN | C | 204 | −32.795 | 64.049 | −19.736 | 1.00 | 52.85 | C |
| ATOM | 7104 | CG | GLN | C | 204 | −33.306 | 63.960 | −18.312 | 1.00 | 77.30 | C |
| ATOM | 7107 | CD | GLN | C | 204 | −34.786 | 64.206 | −18.193 | 1.00 | 79.03 | C |
| ATOM | 7108 | OE1 | GLN | C | 204 | −35.269 | 65.315 | −18.476 | 1.00 | 56.10 | O |
| ATOM | 7109 | NE2 | GLN | C | 204 | −35.514 | 63.167 | −17.793 | 1.00 | 76.36 | N |
| ATOM | 7112 | C | GLN | C | 204 | −31.539 | 61.911 | −19.726 | 1.00 | 55.15 | C |
| ATOM | 7113 | O | GLN | C | 204 | −32.126 | 61.285 | −20.599 | 1.00 | 58.12 | O |
| ATOM | 7114 | N | LEU | C | 205 | −30.902 | 61.312 | −18.728 | 1.00 | 50.00 | N |
| ATOM | 7116 | CA | LEU | C | 205 | −31.227 | 59.927 | −18.449 | 1.00 | 44.80 | C |
| ATOM | 7118 | CB | LEU | C | 205 | −30.231 | 58.975 | −19.095 | 1.00 | 45.30 | C |
| ATOM | 7121 | CG | LEU | C | 205 | −29.797 | 59.084 | −20.554 | 1.00 | 45.89 | C |
| ATOM | 7123 | CD1 | LEU | C | 205 | −28.667 | 60.086 | −20.692 | 1.00 | 51.53 | C |
| ATOM | 7127 | CD2 | LEU | C | 205 | −29.321 | 57.707 | −20.988 | 1.00 | 58.34 | C |
| ATOM | 7131 | C | LEU | C | 205 | −31.302 | 59.617 | −16.977 | 1.00 | 41.08 | C |
| ATOM | 7132 | O | LEU | C | 205 | −31.107 | 60.448 | −16.091 | 1.00 | 47.16 | O |
| ATOM | 7133 | N | THR | C | 206 | −31.605 | 58.367 | −16.721 | 1.00 | 39.96 | N |
| ATOM | 7135 | CA | THR | C | 206 | −31.966 | 57.959 | −15.386 | 1.00 | 42.51 | C |
| ATOM | 7137 | CB | THR | C | 206 | −33.493 | 57.731 | −15.303 | 1.00 | 40.32 | C |
| ATOM | 7139 | OG1 | THR | C | 206 | −34.200 | 58.930 | −15.670 | 1.00 | 35.81 | O |
| ATOM | 7141 | CG2 | THR | C | 206 | −33.882 | 57.479 | −13.862 | 1.00 | 32.99 | C |
| ATOM | 7145 | C | THR | C | 206 | −31.221 | 56.666 | −15.096 | 1.00 | 40.41 | C |
| ATOM | 7146 | O | THR | C | 206 | −31.200 | 55.756 | −15.912 | 1.00 | 33.71 | O |
| ATOM | 7147 | N | PHE | C | 207 | −30.558 | 56.606 | −13.956 | 1.00 | 40.70 | N |
| ATOM | 7149 | CA | PHE | C | 207 | −29.715 | 55.460 | −13.681 | 1.00 | 41.56 | C |
| ATOM | 7151 | CB | PHE | C | 207 | −28.223 | 55.765 | −13.918 | 1.00 | 48.41 | C |
| ATOM | 7154 | CG | PHE | C | 207 | −27.917 | 56.305 | −15.288 | 1.00 | 43.88 | C |
| ATOM | 7155 | CD1 | PHE | C | 207 | −27.829 | 55.459 | −16.370 | 1.00 | 46.55 | C |
| ATOM | 7157 | CE1 | PHE | C | 207 | −27.563 | 55.956 | −17.630 | 1.00 | 44.84 | C |
| ATOM | 7159 | CZ | PHE | C | 207 | −27.356 | 57.301 | −17.812 | 1.00 | 41.58 | C |
| ATOM | 7161 | CE2 | PHE | C | 207 | −27.406 | 58.144 | −16.738 | 1.00 | 46.23 | C |
| ATOM | 7163 | CD2 | PHE | C | 207 | −27.708 | 57.649 | −15.484 | 1.00 | 43.51 | C |
| ATOM | 7165 | C | PHE | C | 207 | −29.919 | 55.244 | −12.218 | 1.00 | 42.98 | C |
| ATOM | 7166 | O | PHE | C | 207 | −30.476 | 56.099 | −11.518 | 1.00 | 56.72 | O |
| ATOM | 7167 | N | ALA | C | 208 | −29.369 | 54.140 | −11.752 | 1.00 | 32.08 | N |
| ATOM | 7169 | CA | ALA | C | 208 | −29.633 | 53.655 | −10.413 | 1.00 | 36.53 | C |
| ATOM | 7171 | CB | ALA | C | 208 | −29.785 | 52.136 | −10.467 | 1.00 | 38.91 | C |
| ATOM | 7175 | C | ALA | C | 208 | −28.453 | 54.015 | −9.530 | 1.00 | 38.88 | C |
| ATOM | 7176 | O | ALA | C | 208 | −27.345 | 53.605 | −9.810 | 1.00 | 40.65 | O |
| ATOM | 7177 | N | LEU | C | 209 | −28.662 | 54.738 | −8.438 | 1.00 | 44.67 | N |
| ATOM | 7179 | CA | LEU | C | 209 | −27.519 | 55.286 | −7.729 | 1.00 | 35.62 | C |
| ATOM | 7181 | CB | LEU | C | 209 | −27.960 | 56.259 | −6.659 | 1.00 | 35.16 | C |
| ATOM | 7184 | CG | LEU | C | 209 | −28.443 | 57.588 | −7.210 | 1.00 | 34.43 | C |
| ATOM | 7186 | CD1 | LEU | C | 209 | −29.353 | 58.277 | −6.179 | 1.00 | 51.54 | C |
| ATOM | 7190 | CD2 | LEU | C | 209 | −27.230 | 58.428 | −7.456 | 1.00 | 41.90 | C |
| ATOM | 7194 | C | LEU | C | 209 | −26.758 | 54.138 | −7.103 | 1.00 | 37.42 | C |
| ATOM | 7195 | O | LEU | C | 209 | −25.540 | 54.209 | −6.968 | 1.00 | 46.85 | O |
| ATOM | 7196 | N | ARG | C | 210 | −27.468 | 53.061 | −6.786 | 1.00 | 39.73 | N |
| ATOM | 7198 | CA | ARG | C | 210 | −26.856 | 51.864 | −6.207 | 1.00 | 41.33 | C |
| ATOM | 7200 | CB | ARG | C | 210 | −27.856 | 50.687 | −6.221 | 1.00 | 48.62 | C |
| ATOM | 7203 | CG | ARG | C | 210 | −27.287 | 49.328 | −6.654 | 1.00 | 71.83 | C |
| ATOM | 7206 | CD | ARG | C | 210 | −28.058 | 48.105 | −6.103 | 1.00 | 82.17 | C |
| ATOM | 7209 | NE | ARG | C | 210 | −28.068 | 46.882 | −6.933 | 1.00 | 77.40 | N |
| ATOM | 7211 | CZ | ARG | C | 210 | −27.088 | 45.960 | −6.988 | 1.00 | 88.33 | C |
| ATOM | 7212 | NH1 | ARG | C | 210 | −25.948 | 46.142 | −6.312 | 1.00 | 79.57 | N |
| ATOM | 7215 | NH2 | ARG | C | 210 | −27.240 | 44.851 | −7.725 | 1.00 | 66.85 | N |
| ATOM | 7218 | C | ARG | C | 210 | −25.544 | 51.543 | −6.913 | 1.00 | 42.75 | C |
| ATOM | 7219 | O | ARG | C | 210 | −24.532 | 51.372 | −6.243 | 1.00 | 48.98 | O |
| ATOM | 7220 | N | TYR | C | 211 | −25.537 | 51.600 | −8.246 | 1.00 | 44.57 | N |
| ATOM | 7222 | CA | TYR | C | 211 | −24.389 | 51.193 | −9.074 | 1.00 | 43.25 | C |
| ATOM | 7224 | CB | TYR | C | 211 | −24.835 | 50.748 | −10.484 | 1.00 | 40.81 | C |
| ATOM | 7227 | CG | TYR | C | 211 | −25.671 | 49.477 | −10.556 | 1.00 | 39.27 | C |
| ATOM | 7228 | CD1 | TYR | C | 211 | −27.056 | 49.509 | −10.429 | 1.00 | 51.94 | C |
| ATOM | 7230 | CE1 | TYR | C | 211 | −27.821 | 48.353 | −10.557 | 1.00 | 34.63 | C |
| ATOM | 7232 | CZ | TYR | C | 211 | −27.196 | 47.162 | −10.801 | 1.00 | 31.18 | C |
| ATOM | 7233 | OH | TYR | C | 211 | −27.887 | 45.973 | −10.824 | 1.00 | 40.29 | O |
| ATOM | 7235 | CE2 | TYR | C | 211 | −25.832 | 47.111 | −10.937 | 1.00 | 40.50 | C |
| ATOM | 7237 | CD2 | TYR | C | 211 | −25.085 | 48.255 | −10.826 | 1.00 | 28.13 | C |
| ATOM | 7239 | C | TYR | C | 211 | −23.327 | 52.282 | −9.262 | 1.00 | 43.80 | C |
| ATOM | 7240 | O | TYR | C | 211 | −22.165 | 51.947 | −9.458 | 1.00 | 45.23 | O |
| ATOM | 7241 | N | LEU | C | 212 | −23.731 | 53.554 | −9.310 | 1.00 | 38.37 | N |
| ATOM | 7243 | CA | LEU | C | 212 | −22.799 | 54.676 | −9.374 | 1.00 | 36.24 | C |
| ATOM | 7245 | CB | LEU | C | 212 | −23.496 | 56.011 | −9.536 | 1.00 | 34.03 | C |
| ATOM | 7248 | CG | LEU | C | 212 | −24.339 | 56.116 | −10.797 | 1.00 | 35.14 | C |
| ATOM | 7250 | CD1 | LEU | C | 212 | −25.010 | 57.467 | −10.649 | 1.00 | 47.79 | C |
| ATOM | 7254 | CD2 | LEU | C | 212 | −23.484 | 56.029 | −12.068 | 1.00 | 26.02 | C |
| ATOM | 7258 | C | LEU | C | 212 | −21.980 | 54.805 | −8.117 | 1.00 | 40.00 | C |
| ATOM | 7259 | O | LEU | C | 212 | −20.872 | 55.336 | −8.134 | 1.00 | 41.36 | O |
| ATOM | 7260 | N | ASN | C | 213 | −22.509 | 54.294 | −7.023 | 1.00 | 39.30 | N |
| ATOM | 7262 | CA | ASN | C | 213 | −21.767 | 54.417 | −5.785 | 1.00 | 42.82 | C |
| ATOM | 7264 | CB | ASN | C | 213 | −22.727 | 54.321 | −4.608 | 1.00 | 47.21 | C |
| ATOM | 7267 | CG | ASN | C | 213 | −23.332 | 55.654 | −4.289 | 1.00 | 48.05 | C |
| ATOM | 7268 | OD1 | ASN | C | 213 | −22.623 | 56.671 | −4.330 | 1.00 | 42.64 | O |
| ATOM | 7269 | ND2 | ASN | C | 213 | −24.638 | 55.673 | −4.003 | 1.00 | 42.12 | N |
| ATOM | 7272 | C | ASN | C | 213 | −20.740 | 53.338 | −5.637 | 1.00 | 42.04 | C |
| ATOM | 7273 | O | ASN | C | 213 | −19.833 | 53.477 | −4.847 | 1.00 | 47.71 | O |
| ATOM | 7274 | N | PHE | C | 214 | −20.980 | 52.194 | −6.261 | 1.00 | 45.45 | N |
| ATOM | 7276 | CA | PHE | C | 214 | −19.896 | 51.281 | −6.537 | 1.00 | 39.13 | C |
| ATOM | 7278 | CB | PHE | C | 214 | −20.426 | 50.018 | −7.162 | 1.00 | 35.85 | C |
| ATOM | 7281 | CG | PHE | C | 214 | −20.936 | 49.067 | −6.168 | 1.00 | 31.25 | C |
| ATOM | 7282 | CD1 | PHE | C | 214 | −20.078 | 48.198 | −5.524 | 1.00 | 41.85 | C |
| ATOM | 7284 | CE1 | PHE | C | 214 | −20.541 | 47.347 | −4.525 | 1.00 | 37.56 | C |
| ATOM | 7286 | CZ | PHE | C | 214 | −21.890 | 47.333 | −4.216 | 1.00 | 52.37 | C |
| ATOM | 7288 | CE2 | PHE | C | 214 | −22.771 | 48.176 | −4.904 | 1.00 | 35.23 | C |
| ATOM | 7290 | CD2 | PHE | C | 214 | −22.281 | 49.029 | −5.872 | 1.00 | 48.92 | C |
| ATOM | 7292 | C | PHE | C | 214 | −18.811 | 51.901 | −7.423 | 1.00 | 39.54 | C |
| ATOM | 7293 | O | PHE | C | 214 | −17.654 | 51.671 | −7.157 | 1.00 | 44.06 | O |
| ATOM | 7294 | N | PHE | C | 215 | −19.157 | 52.705 | −8.422 | 1.00 | 37.88 | N |
| ATOM | 7296 | CA | PHE | C | 215 | −18.188 | 53.168 | −9.406 | 1.00 | 41.27 | C |
| ATOM | 7298 | CB | PHE | C | 215 | −18.869 | 53.922 | −10.562 | 1.00 | 50.26 | C |
| ATOM | 7301 | CG | PHE | C | 215 | −19.730 | 53.057 | −11.450 | 1.00 | 39.24 | C |
| ATOM | 7302 | CD1 | PHE | C | 215 | −19.655 | 51.679 | −11.374 | 1.00 | 41.73 | C |
| ATOM | 7304 | CE1 | PHE | C | 215 | −20.452 | 50.877 | −12.170 | 1.00 | 36.27 | C |
| ATOM | 7306 | CZ | PHE | C | 215 | −21.322 | 51.441 | −13.073 | 1.00 | 35.98 | C |
| ATOM | 7308 | CE2 | PHE | C | 215 | −21.377 | 52.833 | −13.206 | 1.00 | 45.37 | C |
| ATOM | 7310 | CD2 | PHE | C | 215 | −20.578 | 53.633 | −12.394 | 1.00 | 42.00 | C |
| ATOM | 7312 | C | PHE | C | 215 | −17.270 | 54.152 | −8.714 | 1.00 | 38.00 | C |
| ATOM | 7313 | O | PHE | C | 215 | −16.073 | 54.202 | −8.974 | 1.00 | 40.46 | O |
| ATOM | 7314 | N | THR | C | 216 | −17.852 | 54.964 | −7.851 | 1.00 | 34.94 | N |
| ATOM | 7316 | CA | THR | C | 216 | −17.069 | 55.982 | −7.161 | 1.00 | 37.92 | C |
| ATOM | 7318 | CB | THR | C | 216 | −17.973 | 57.004 | −6.419 | 1.00 | 29.45 | C |
| ATOM | 7320 | OG1 | THR | C | 216 | −19.028 | 56.378 | −5.667 | 1.00 | 45.78 | O |
| ATOM | 7322 | CG2 | THR | C | 216 | −18.648 | 57.884 | −7.415 | 1.00 | 29.85 | C |
| ATOM | 7326 | C | THR | C | 216 | −15.970 | 55.432 | −6.230 | 1.00 | 39.27 | C |
| ATOM | 7327 | O | THR | C | 216 | −15.139 | 56.165 | −5.682 | 1.00 | 33.71 | O |
| ATOM | 7328 | N | LYS | C | 217 | −15.889 | 54.120 | −6.129 | 1.00 | 36.61 | N |
| ATOM | 7330 | CA | LYS | C | 217 | −14.775 | 53.539 | −5.431 | 1.00 | 29.83 | C |
| ATOM | 7332 | CB | LYS | C | 217 | −15.179 | 52.119 | −5.095 | 1.00 | 29.37 | C |
| ATOM | 7335 | CG | LYS | C | 217 | −15.678 | 52.087 | −3.702 | 1.00 | 41.28 | C |
| ATOM | 7338 | CD | LYS | C | 217 | −16.901 | 51.206 | −3.487 | 1.00 | 42.98 | C |
| ATOM | 7341 | CE | LYS | C | 217 | −17.211 | 51.269 | −1.972 | 1.00 | 64.36 | C |
| ATOM | 7344 | NZ | LYS | C | 217 | −18.575 | 51.756 | −1.594 | 1.00 | 64.38 | N |
| ATOM | 7348 | C | LYS | C | 217 | −13.490 | 53.601 | −6.280 | 1.00 | 39.80 | C |
| ATOM | 7349 | O | LYS | C | 217 | −12.381 | 53.296 | −5.797 | 1.00 | 40.96 | O |
| ATOM | 7350 | N | ALA | C | 218 | −13.597 | 54.060 | −7.526 | 1.00 | 34.87 | N |
| ATOM | 7352 | CA | ALA | C | 218 | −12.394 | 54.547 | −8.211 | 1.00 | 33.85 | C |
| ATOM | 7354 | CB | ALA | C | 218 | −12.541 | 54.308 | −9.642 | 1.00 | 41.01 | C |
| ATOM | 7358 | C | ALA | C | 218 | −11.868 | 55.979 | −7.999 | 1.00 | 35.79 | C |
| ATOM | 7359 | O | ALA | C | 218 | −10.950 | 56.415 | −8.703 | 1.00 | 38.60 | O |
| ATOM | 7360 | N | THR | C | 219 | −12.414 | 56.707 | −7.035 | 1.00 | 29.44 | N |
| ATOM | 7362 | CA | THR | C | 219 | −12.130 | 58.125 | −6.937 | 1.00 | 29.84 | C |
| ATOM | 7364 | CB | THR | C | 219 | −13.015 | 58.744 | −5.864 | 1.00 | 32.06 | C |
| ATOM | 7366 | OG1 | THR | C | 219 | −14.400 | 58.637 | −6.229 | 1.00 | 33.23 | O |
| ATOM | 7368 | CG2 | THR | C | 219 | −12.829 | 60.198 | −5.757 | 1.00 | 30.06 | C |
| ATOM | 7372 | C | THR | C | 219 | −10.653 | 58.386 | −6.660 | 1.00 | 28.68 | C |
| ATOM | 7373 | O | THR | C | 219 | −10.049 | 59.314 | −7.208 | 1.00 | 35.93 | O |
| ATOM | 7374 | N | PRO | C | 220 | −10.028 | 57.527 | −5.876 | 1.00 | 26.78 | N |
| ATOM | 7375 | CA | PRO | C | 220 | −8.605 | 57.702 | −5.553 | 1.00 | 26.63 | C |
| ATOM | 7377 | CB | PRO | C | 220 | −8.333 | 56.578 | −4.568 | 1.00 | 17.97 | C |
| ATOM | 7380 | CG | PRO | C | 220 | −9.650 | 56.302 | −3.999 | 1.00 | 31.11 | C |
| ATOM | 7383 | CD | PRO | C | 220 | −10.599 | 56.358 | −5.198 | 1.00 | 33.97 | C |
| ATOM | 7386 | C | PRO | C | 220 | −7.687 | 57.598 | −6.764 | 1.00 | 26.35 | C |
| ATOM | 7387 | O | PRO | C | 220 | −6.549 | 58.046 | −6.699 | 1.00 | 33.11 | O |
| ATOM | 7388 | N | LEU | C | 221 | −8.186 | 57.066 | −7.876 | 1.00 | 32.54 | N |
| ATOM | 7390 | CA | LEU | C | 221 | −7.335 | 56.854 | −9.045 | 1.00 | 26.32 | C |
| ATOM | 7392 | CB | LEU | C | 221 | −7.902 | 55.832 | −10.017 | 1.00 | 13.51 | C |
| ATOM | 7395 | CG | LEU | C | 221 | −7.767 | 54.355 | −9.626 | 1.00 | 44.91 | C |
| ATOM | 7397 | CD1 | LEU | C | 221 | −8.839 | 53.552 | −10.257 | 1.00 | 41.99 | C |
| ATOM | 7401 | CD2 | LEU | C | 221 | −6.423 | 53.710 | −9.920 | 1.00 | 44.04 | C |
| ATOM | 7405 | C | LEU | C | 221 | −7.219 | 58.162 | −9.769 | 1.00 | 29.71 | C |
| ATOM | 7406 | O | LEU | C | 221 | −6.256 | 58.368 | −10.494 | 1.00 | 42.31 | O |
| ATOM | 7407 | N | SER | C | 222 | −8.195 | 59.044 | −9.623 | 1.00 | 26.69 | N |
| ATOM | 7409 | CA | SER | C | 222 | −8.043 | 60.303 | −10.330 | 1.00 | 29.13 | C |
| ATOM | 7411 | CB | SER | C | 222 | −8.601 | 60.209 | −11.736 | 1.00 | 33.49 | C |
| ATOM | 7414 | OG | SER | C | 222 | −8.413 | 61.454 | −12.369 | 1.00 | 38.77 | O |
| ATOM | 7416 | C | SER | C | 222 | −8.829 | 61.374 | −9.690 | 1.00 | 29.65 | C |
| ATOM | 7417 | O | SER | C | 222 | −9.910 | 61.140 | −9.135 | 1.00 | 40.27 | O |
| ATOM | 7418 | N | SER | C | 223 | −8.344 | 62.584 | −9.824 | 1.00 | 24.43 | N |
| ATOM | 7420 | CA | SER | C | 223 | −9.116 | 63.682 | −9.226 | 1.00 | 34.31 | C |
| ATOM | 7422 | CB | SER | C | 223 | −8.231 | 64.869 | −8.875 | 1.00 | 23.32 | C |
| ATOM | 7425 | OG | SER | C | 223 | −7.974 | 65.468 | −10.126 | 1.00 | 38.58 | O |
| ATOM | 7427 | C | SER | C | 223 | −10.220 | 64.207 | −10.161 | 1.00 | 36.64 | C |
| ATOM | 7428 | O | SER | C | 223 | −11.070 | 64.984 | −9.736 | 1.00 | 44.91 | O |
| ATOM | 7429 | N | THR | C | 224 | −10.216 | 63.801 | −11.420 | 1.00 | 34.96 | N |
| ATOM | 7431 | CA | THR | C | 224 | −11.296 | 64.184 | −12.297 | 1.00 | 45.58 | C |
| ATOM | 7433 | CB | THR | C | 224 | −10.777 | 65.097 | −13.437 | 1.00 | 45.89 | C |
| ATOM | 7435 | OG1 | THR | C | 224 | −10.773 | 64.375 | −14.682 | 1.00 | 44.14 | O |
| ATOM | 7437 | CG2 | THR | C | 224 | −9.353 | 65.488 | −13.205 | 1.00 | 57.14 | C |
| ATOM | 7441 | C | THR | C | 224 | −11.957 | 62.923 | −12.858 | 1.00 | 42.44 | C |
| ATOM | 7442 | O | THR | C | 224 | −11.248 | 61.945 | −13.050 | 1.00 | 38.25 | O |
| ATOM | 7443 | N | VAL | C | 225 | −13.278 | 62.972 | −13.105 | 1.00 | 33.10 | N |
| ATOM | 7445 | CA | VAL | C | 225 | −14.028 | 61.924 | −13.802 | 1.00 | 27.88 | C |
| ATOM | 7447 | CB | VAL | C | 225 | −15.068 | 61.253 | −12.834 | 1.00 | 33.33 | C |
| ATOM | 7449 | CG1 | VAL | C | 225 | −16.176 | 62.222 | −12.460 | 1.00 | 34.93 | C |
| ATOM | 7453 | CG2 | VAL | C | 225 | −15.720 | 59.979 | −13.424 | 1.00 | 26.80 | C |
| ATOM | 7457 | C | VAL | C | 225 | −14.764 | 62.485 | −15.029 | 1.00 | 30.39 | C |
| ATOM | 7458 | O | VAL | C | 225 | −15.231 | 63.614 | −15.019 | 1.00 | 33.15 | O |
| ATOM | 7459 | N | THR | C | 226 | −14.852 | 61.717 | −16.111 | 1.00 | 36.83 | N |
| ATOM | 7461 | CA | THR | C | 226 | −15.767 | 62.079 | −17.186 | 1.00 | 43.22 | C |
| ATOM | 7463 | CB | THR | C | 226 | −15.107 | 62.273 | −18.569 | 1.00 | 38.09 | C |
| ATOM | 7465 | OG1 | THR | C | 226 | −15.330 | 61.106 | −19.361 | 1.00 | 55.94 | O |
| ATOM | 7467 | CG2 | THR | C | 226 | −13.596 | 62.496 | −18.461 | 1.00 | 52.98 | C |
| ATOM | 7471 | C | THR | C | 226 | −17.013 | 61.202 | −17.285 | 1.00 | 41.58 | C |
| ATOM | 7472 | O | THR | C | 226 | −16.949 | 59.990 | −17.080 | 1.00 | 38.86 | O |
| ATOM | 7473 | N | LEU | C | 227 | −18.132 | 61.889 | −17.549 | 1.00 | 36.50 | N |
| ATOM | 7475 | CA | LEU | C | 227 | −19.431 | 61.297 | −17.825 | 1.00 | 42.26 | C |
| ATOM | 7477 | CB | LEU | C | 227 | −20.532 | 61.978 | −17.017 | 1.00 | 37.23 | C |
| ATOM | 7480 | CG | LEU | C | 227 | −20.358 | 62.066 | −15.500 | 1.00 | 37.60 | C |
| ATOM | 7482 | CD1 | LEU | C | 227 | −21.695 | 62.238 | −14.858 | 1.00 | 35.22 | C |
| ATOM | 7486 | CD2 | LEU | C | 227 | −19.641 | 60.872 | −14.882 | 1.00 | 46.73 | C |
| ATOM | 7490 | C | LEU | C | 227 | −19.699 | 61.551 | −19.299 | 1.00 | 44.30 | C |
| ATOM | 7491 | O | LEU | C | 227 | −19.689 | 62.698 | −19.739 | 1.00 | 53.02 | O |
| ATOM | 7492 | N | SER | C | 228 | −19.896 | 60.457 | −20.029 | 1.00 | 41.82 | N |
| ATOM | 7494 | CA | SER | C | 228 | −20.342 | 60.440 | −21.416 | 1.00 | 40.04 | C |
| ATOM | 7496 | CB | SER | C | 228 | −19.345 | 59.565 | −22.187 | 1.00 | 46.01 | C |
| ATOM | 7499 | OG | SER | C | 228 | −18.098 | 60.237 | −22.311 | 1.00 | 28.26 | O |
| ATOM | 7501 | C | SER | C | 228 | −21.761 | 59.853 | −21.568 | 1.00 | 40.02 | C |
| ATOM | 7502 | O | SER | C | 228 | −22.012 | 58.770 | −21.060 | 1.00 | 40.00 | O |
| ATOM | 7503 | N | MET | C | 229 | −22.693 | 60.582 | −22.189 | 1.00 | 48.02 | N |
| ATOM | 7505 | CA | MET | C | 229 | −24.139 | 60.250 | −22.220 | 1.00 | 45.50 | C |
| ATOM | 7507 | CB | MET | C | 229 | −24.938 | 61.151 | −21.287 | 1.00 | 42.94 | C |
| ATOM | 7510 | CG | MET | C | 229 | −24.789 | 60.750 | −19.836 | 1.00 | 56.36 | C |
| ATOM | 7513 | SD | MET | C | 229 | −25.334 | 61.979 | −18.661 | 1.00 | 48.77 | S |
| ATOM | 7514 | CE | MET | C | 229 | −24.245 | 63.434 | −19.030 | 1.00 | 54.36 | C |
| ATOM | 7518 | C | MET | C | 229 | −24.743 | 60.427 | −23.610 | 1.00 | 41.05 | C |
| ATOM | 7519 | O | MET | C | 229 | −24.405 | 61.347 | −24.346 | 1.00 | 34.76 | O |
| ATOM | 7520 | N | SER | C | 230 | −25.660 | 59.536 | −23.947 | 1.00 | 45.51 | N |
| ATOM | 7522 | CA | SER | C | 230 | −26.525 | 59.666 | −25.121 | 1.00 | 48.13 | C |
| ATOM | 7524 | CB | SER | C | 230 | −25.861 | 59.092 | −26.368 | 1.00 | 45.92 | C |
| ATOM | 7527 | OG | SER | C | 230 | −25.320 | 60.118 | −27.169 | 1.00 | 45.92 | O |
| ATOM | 7529 | C | SER | C | 230 | −27.818 | 58.896 | −24.870 | 1.00 | 44.62 | C |
| ATOM | 7530 | O | SER | C | 230 | −27.859 | 57.945 | −24.104 | 1.00 | 45.65 | O |
| ATOM | 7531 | N | ALA | C | 231 | −28.898 | 59.312 | −25.497 | 1.00 | 44.26 | N |
| ATOM | 7533 | CA | ALA | C | 231 | −30.159 | 58.691 | −25.158 | 1.00 | 43.31 | C |
| ATOM | 7535 | CB | ALA | C | 231 | −31.280 | 59.445 | −25.815 | 1.00 | 45.16 | C |
| ATOM | 7539 | C | ALA | C | 231 | −30.189 | 57.198 | −25.516 | 1.00 | 40.62 | C |
| ATOM | 7540 | O | ALA | C | 231 | −29.677 | 56.773 | −26.553 | 1.00 | 40.70 | O |
| ATOM | 7541 | N | ASP | C | 232 | −30.781 | 56.422 | −24.610 | 1.00 | 45.64 | N |
| ATOM | 7543 | CA | ASP | C | 232 | −30.913 | 54.957 | −24.700 | 1.00 | 50.21 | C |
| ATOM | 7545 | CB | ASP | C | 232 | −32.104 | 54.546 | −25.598 | 1.00 | 48.44 | C |
| ATOM | 7548 | CG | ASP | C | 232 | −33.207 | 55.646 | −25.690 | 1.00 | 71.80 | C |
| ATOM | 7549 | OD1 | ASP | C | 232 | −33.507 | 56.300 | −24.662 | 1.00 | 101.37 | O |
| ATOM | 7550 | OD2 | ASP | C | 232 | −33.868 | 55.935 | −26.718 | 1.00 | 70.02 | O |
| ATOM | 7551 | C | ASP | C | 232 | −29.578 | 54.310 | −25.085 | 1.00 | 45.70 | C |
| ATOM | 7552 | O | ASP | C | 232 | −29.463 | 53.503 | −26.005 | 1.00 | 52.44 | O |
| ATOM | 7553 | N | VAL | C | 233 | −28.544 | 54.712 | −24.367 | 1.00 | 44.53 | N |
| ATOM | 7555 | CA | VAL | C | 233 | −27.322 | 53.917 | −24.279 | 1.00 | 44.15 | C |
| ATOM | 7557 | CB | VAL | C | 233 | −26.266 | 54.381 | −25.307 | 1.00 | 42.55 | C |
| ATOM | 7559 | CG1 | VAL | C | 233 | −26.907 | 54.549 | −26.621 | 1.00 | 18.96 | C |
| ATOM | 7563 | CG2 | VAL | C | 233 | −25.610 | 55.706 | −24.880 | 1.00 | 41.06 | C |
| ATOM | 7567 | C | VAL | C | 233 | −26.658 | 53.979 | −22.900 | 1.00 | 42.72 | C |
| ATOM | 7568 | O | VAL | C | 233 | −26.941 | 54.837 | −22.050 | 1.00 | 45.02 | O |
| ATOM | 7569 | N | PRO | C | 234 | −25.760 | 53.038 | −22.680 | 1.00 | 37.73 | N |
| ATOM | 7570 | CA | PRO | C | 234 | −25.104 | 52.977 | −21.377 | 1.00 | 42.45 | C |
| ATOM | 7572 | CB | PRO | C | 234 | −24.292 | 51.695 | −21.449 | 1.00 | 40.29 | C |
| ATOM | 7575 | CG | PRO | C | 234 | −24.910 | 50.928 | −22.617 | 1.00 | 36.64 | C |
| ATOM | 7578 | CD | PRO | C | 234 | −25.412 | 51.919 | −23.571 | 1.00 | 28.43 | C |
| ATOM | 7581 | C | PRO | C | 234 | −24.256 | 54.212 | −21.188 | 1.00 | 40.67 | C |
| ATOM | 7582 | O | PRO | C | 234 | −23.641 | 54.680 | −22.151 | 1.00 | 47.13 | O |
| ATOM | 7583 | N | LEU | C | 235 | −24.316 | 54.758 | −19.981 | 1.00 | 37.08 | N |
| ATOM | 7585 | CA | LEU | C | 235 | −23.520 | 55.911 | −19.611 | 1.00 | 39.51 | C |
| ATOM | 7587 | CB | LEU | C | 235 | −24.091 | 56.580 | −18.361 | 1.00 | 32.11 | C |
| ATOM | 7590 | CG | LEU | C | 235 | −23.057 | 57.094 | −17.360 | 1.00 | 40.08 | C |
| ATOM | 7592 | CD1 | LEU | C | 235 | −22.745 | 58.539 | −17.681 | 1.00 | 52.44 | C |
| ATOM | 7596 | CD2 | LEU | C | 235 | −23.606 | 57.027 | −15.961 | 1.00 | 43.61 | C |
| ATOM | 7600 | C | LEU | C | 235 | −22.121 | 55.397 | −19.323 | 1.00 | 42.42 | C |
| ATOM | 7601 | O | LEU | C | 235 | −22.008 | 54.273 | −18.859 | 1.00 | 48.12 | O |
| ATOM | 7602 | N | VAL | C | 236 | −21.089 | 56.206 | −19.580 | 1.00 | 38.19 | N |
| ATOM | 7604 | CA | VAL | C | 236 | −19.720 | 55.809 | −19.300 | 1.00 | 39.28 | C |
| ATOM | 7606 | CB | VAL | C | 236 | −18.798 | 55.880 | −20.512 | 1.00 | 44.24 | C |
| ATOM | 7608 | CG1 | VAL | C | 236 | −17.536 | 55.133 | −20.216 | 1.00 | 47.54 | C |
| ATOM | 7612 | CG2 | VAL | C | 236 | −19.442 | 55.229 | −21.729 | 1.00 | 56.53 | C |
| ATOM | 7616 | C | VAL | C | 236 | −19.176 | 56.728 | −18.260 | 1.00 | 39.94 | C |
| ATOM | 7617 | O | VAL | C | 236 | −19.277 | 57.957 | −18.351 | 1.00 | 45.35 | O |
| ATOM | 7618 | N | VAL | C | 237 | −18.655 | 56.089 | −17.229 | 1.00 | 37.65 | N |
| ATOM | 7620 | CA | VAL | C | 237 | −18.002 | 56.795 | −16.140 | 1.00 | 36.23 | C |
| ATOM | 7622 | CB | VAL | C | 237 | −18.667 | 56.417 | −14.808 | 1.00 | 42.77 | C |
| ATOM | 7624 | CG1 | VAL | C | 237 | −18.122 | 57.251 | −13.670 | 1.00 | 35.19 | C |
| ATOM | 7628 | CG2 | VAL | C | 237 | −20.180 | 56.593 | −14.915 | 1.00 | 40.88 | C |
| ATOM | 7632 | C | VAL | C | 237 | −16.526 | 56.414 | −16.155 | 1.00 | 32.76 | C |
| ATOM | 7633 | O | VAL | C | 237 | −16.190 | 55.267 | −15.845 | 1.00 | 33.90 | O |
| ATOM | 7634 | N | GLU | C | 238 | −15.674 | 57.340 | −16.617 | 1.00 | 36.59 | N |
| ATOM | 7636 | CA | GLU | C | 238 | −14.256 | 57.079 | −16.910 | 1.00 | 34.20 | C |
| ATOM | 7638 | CB | GLU | C | 238 | −13.883 | 57.669 | −18.251 | 1.00 | 38.37 | C |
| ATOM | 7641 | CG | GLU | C | 238 | −12.560 | 57.189 | −18.825 | 1.00 | 53.51 | C |
| ATOM | 7644 | CD | GLU | C | 238 | −12.272 | 57.783 | −20.187 | 1.00 | 51.52 | C |
| ATOM | 7645 | OE1 | GLU | C | 238 | −11.072 | 57.938 | −20.519 | 1.00 | 80.24 | O |
| ATOM | 7646 | OE2 | GLU | C | 238 | −13.262 | 58.082 | −20.895 | 1.00 | 65.64 | O |
| ATOM | 7647 | C | GLU | C | 238 | −13.346 | 57.704 | −15.857 | 1.00 | 36.87 | C |
| ATOM | 7648 | O | GLU | C | 238 | −13.479 | 58.896 | −15.572 | 1.00 | 43.57 | O |
| ATOM | 7649 | N | TYR | C | 239 | −12.433 | 56.925 | −15.274 | 1.00 | 33.61 | N |
| ATOM | 7651 | CA | TYR | C | 239 | −11.405 | 57.499 | −14.403 | 1.00 | 33.47 | C |
| ATOM | 7653 | CB | TYR | C | 239 | −11.456 | 56.841 | −13.019 | 1.00 | 25.50 | C |
| ATOM | 7656 | CG | TYR | C | 239 | −12.715 | 57.092 | −12.247 | 1.00 | 37.83 | C |
| ATOM | 7657 | CD1 | TYR | C | 239 | −13.812 | 56.236 | −12.315 | 1.00 | 32.57 | C |
| ATOM | 7659 | CE1 | TYR | C | 239 | −14.925 | 56.478 | −11.534 | 1.00 | 32.96 | C |
| ATOM | 7661 | CZ | TYR | C | 239 | −14.994 | 57.623 | −10.747 | 1.00 | 47.12 | C |
| ATOM | 7662 | OH | TYR | C | 239 | −16.095 | 57.982 | −9.968 | 1.00 | 40.14 | O |
| ATOM | 7664 | CE2 | TYR | C | 239 | −13.908 | 58.445 | −10.675 | 1.00 | 42.24 | C |
| ATOM | 7666 | CD2 | TYR | C | 239 | −12.776 | 58.155 | −11.381 | 1.00 | 30.39 | C |
| ATOM | 7668 | C | TYR | C | 239 | −10.042 | 57.207 | −15.056 | 1.00 | 33.30 | C |
| ATOM | 7669 | O | TYR | C | 239 | −9.749 | 56.025 | −15.278 | 1.00 | 37.37 | O |
| ATOM | 7670 | N | LYS | C | 240 | −9.212 | 58.230 | −15.322 | 1.00 | 35.26 | N |
| ATOM | 7672 | CA | LYS | C | 240 | −7.926 | 58.008 | −16.029 | 1.00 | 40.49 | C |
| ATOM | 7674 | CB | LYS | C | 240 | −7.500 | 59.128 | −17.001 | 1.00 | 40.67 | C |
| ATOM | 7677 | CG | LYS | C | 240 | −8.560 | 59.348 | −18.207 | 1.00 | 62.53 | C |
| ATOM | 7680 | CD | LYS | C | 240 | −8.035 | 59.676 | −19.645 | 1.00 | 68.33 | C |
| ATOM | 7683 | CE | LYS | C | 240 | −7.960 | 58.424 | −20.597 | 1.00 | 88.06 | C |
| ATOM | 7686 | NZ | LYS | C | 240 | −6.712 | 58.217 | −21.477 | 1.00 | 75.86 | N |
| ATOM | 7690 | C | LYS | C | 240 | −6.878 | 57.685 | −15.011 | 1.00 | 35.07 | C |
| ATOM | 7691 | O | LYS | C | 240 | −6.795 | 58.356 | −14.005 | 1.00 | 55.71 | O |
| ATOM | 7692 | N | ILE | C | 241 | −6.241 | 56.535 | −15.173 | 1.00 | 38.51 | N |
| ATOM | 7694 | CA | ILE | C | 241 | −5.084 | 56.108 | −14.376 | 1.00 | 37.25 | C |
| ATOM | 7696 | CB | ILE | C | 241 | −5.044 | 54.585 | −14.390 | 1.00 | 36.60 | C |
| ATOM | 7698 | CG1 | ILE | C | 241 | −6.344 | 54.004 | −13.835 | 1.00 | 36.75 | C |
| ATOM | 7701 | CD1 | ILE | C | 241 | −6.511 | 52.559 | −14.092 | 1.00 | 30.84 | C |
| ATOM | 7705 | CG2 | ILE | C | 241 | −3.836 | 54.124 | −13.616 | 1.00 | 42.32 | C |
| ATOM | 7709 | C | ILE | C | 241 | −3.733 | 56.559 | −14.933 | 1.00 | 42.30 | C |
| ATOM | 7710 | O | ILE | C | 241 | −3.211 | 55.934 | −15.836 | 1.00 | 43.62 | O |
| ATOM | 7711 | N | ALA | C | 242 | −3.152 | 57.637 | −14.427 | 1.00 | 54.71 | N |
| ATOM | 7713 | CA | ALA | C | 242 | −2.386 | 58.546 | −15.277 | 1.00 | 54.78 | C |
| ATOM | 7715 | CB | ALA | C | 242 | −1.943 | 59.767 | −14.502 | 1.00 | 66.43 | C |
| ATOM | 7719 | C | ALA | C | 242 | −1.152 | 57.850 | −15.750 | 1.00 | 58.25 | C |
| ATOM | 7720 | O | ALA | C | 242 | −0.484 | 57.208 | −14.940 | 1.00 | 56.31 | O |
| ATOM | 7721 | N | ASP | C | 243 | −0.852 | 58.063 | −17.031 | 1.00 | 63.04 | N |
| ATOM | 7723 | CA | ASP | C | 243 | 0.301 | 57.494 | −17.709 | 1.00 | 68.99 | C |
| ATOM | 7725 | CB | ASP | C | 243 | 1.569 | 57.958 | −16.977 | 1.00 | 75.46 | C |
| ATOM | 7728 | CG | ASP | C | 243 | 1.546 | 59.441 | −16.671 | 1.00 | 87.16 | C |
| ATOM | 7729 | OD1 | ASP | C | 243 | 0.648 | 60.130 | −17.211 | 1.00 | 101.71 | O |
| ATOM | 7730 | OD2 | ASP | C | 243 | 2.366 | 59.988 | −15.898 | 1.00 | 94.55 | O |
| ATOM | 7731 | C | ASP | C | 243 | 0.312 | 55.957 | −17.826 | 1.00 | 64.87 | C |
| ATOM | 7732 | O | ASP | C | 243 | 1.352 | 55.319 | −17.655 | 1.00 | 78.76 | O |
| ATOM | 7733 | N | MET | C | 244 | −0.820 | 55.349 | −18.122 | 1.00 | 47.87 | N |
| ATOM | 7735 | CA | MET | C | 244 | −0.991 | 53.922 | −17.933 | 1.00 | 43.55 | C |
| ATOM | 7737 | CB | MET | C | 244 | −1.325 | 53.549 | −16.488 | 1.00 | 39.65 | C |
| ATOM | 7740 | CG | MET | C | 244 | −0.151 | 53.515 | −15.565 | 1.00 | 60.44 | C |
| ATOM | 7743 | SD | MET | C | 244 | 0.127 | 51.927 | −14.870 | 1.00 | 59.67 | S |
| ATOM | 7744 | CE | MET | C | 244 | 0.084 | 50.997 | −16.372 | 1.00 | 71.30 | C |
| ATOM | 7748 | C | MET | C | 244 | −2.192 | 53.554 | −18.753 | 1.00 | 38.71 | C |
| ATOM | 7749 | O | MET | C | 244 | −2.135 | 52.629 | −19.521 | 1.00 | 46.22 | O |
| ATOM | 7750 | N | GLY | C | 245 | −3.309 | 54.211 | −18.469 | 1.00 | 43.32 | N |
| ATOM | 7752 | CA | GLY | C | 245 | −4.608 | 53.888 | −19.023 | 1.00 | 44.15 | C |
| ATOM | 7755 | C | GLY | C | 245 | −5.754 | 54.431 | −18.190 | 1.00 | 41.64 | C |
| ATOM | 7756 | O | GLY | C | 245 | −5.785 | 55.578 | −17.781 | 1.00 | 41.91 | O |
| ATOM | 7757 | N | HIS | C | 246 | −6.772 | 53.621 | −17.996 | 1.00 | 41.66 | N |
| ATOM | 7759 | CA | HIS | C | 246 | −8.056 | 54.178 | −17.610 | 1.00 | 40.02 | C |
| ATOM | 7761 | CB | HIS | C | 246 | −8.703 | 54.891 | −18.788 | 1.00 | 36.85 | C |
| ATOM | 7764 | CG | HIS | C | 246 | −8.948 | 54.004 | −19.969 | 1.00 | 55.92 | C |
| ATOM | 7765 | ND1 | HIS | C | 246 | −8.436 | 54.270 | −21.227 | 1.00 | 28.72 | N |
| ATOM | 7767 | CE1 | HIS | C | 246 | −8.876 | 53.341 | −22.061 | 1.00 | 63.84 | C |
| ATOM | 7769 | NE2 | HIS | C | 246 | −9.578 | 52.447 | −21.383 | 1.00 | 47.30 | N |
| ATOM | 7771 | CD2 | HIS | C | 246 | −9.624 | 52.830 | −20.067 | 1.00 | 54.34 | C |
| ATOM | 7773 | C | HIS | C | 246 | −8.923 | 53.032 | −17.168 | 1.00 | 35.85 | C |
| ATOM | 7774 | O | HIS | C | 246 | −8.640 | 51.863 | −17.424 | 1.00 | 39.17 | O |
| ATOM | 7775 | N | LEU | C | 247 | −9.976 | 53.374 | −16.467 | 1.00 | 35.72 | N |
| ATOM | 7777 | CA | LEU | C | 247 | −10.889 | 52.360 | −15.994 | 1.00 | 34.45 | C |
| ATOM | 7779 | CB | LEU | C | 247 | −10.598 | 52.152 | −14.514 | 1.00 | 37.28 | C |
| ATOM | 7782 | CG | LEU | C | 247 | −11.474 | 51.256 | −13.669 | 1.00 | 37.03 | C |
| ATOM | 7784 | CD1 | LEU | C | 247 | −11.622 | 49.886 | −14.315 | 1.00 | 50.88 | C |
| ATOM | 7788 | CD2 | LEU | C | 247 | −10.798 | 51.198 | −12.309 | 1.00 | 34.28 | C |
| ATOM | 7792 | C | LEU | C | 247 | −12.254 | 52.952 | −16.251 | 1.00 | 37.68 | C |
| ATOM | 7793 | O | LEU | C | 247 | −12.476 | 54.155 | −16.035 | 1.00 | 38.48 | O |
| ATOM | 7794 | N | LYS | C | 248 | −13.104 | 52.159 | −16.879 | 1.00 | 38.23 | N |
| ATOM | 7796 | CA | LYS | C | 248 | −14.356 | 52.685 | −17.425 | 1.00 | 40.71 | C |
| ATOM | 7798 | CB | LYS | C | 248 | −14.393 | 52.482 | −18.935 | 1.00 | 34.49 | C |
| ATOM | 7801 | CG | LYS | C | 248 | −13.984 | 53.703 | −19.699 | 1.00 | 29.56 | C |
| ATOM | 7804 | CD | LYS | C | 248 | −13.798 | 53.456 | −21.192 | 1.00 | 36.23 | C |
| ATOM | 7807 | CE | LYS | C | 248 | −12.818 | 54.525 | −21.692 | 1.00 | 32.66 | C |
| ATOM | 7810 | NZ | LYS | C | 248 | −12.564 | 54.460 | −23.116 | 1.00 | 34.07 | N |
| ATOM | 7814 | C | LYS | C | 248 | −15.493 | 51.877 | −16.840 | 1.00 | 43.44 | C |
| ATOM | 7815 | O | LYS | C | 248 | −15.408 | 50.660 | −16.778 | 1.00 | 49.61 | O |
| ATOM | 7816 | N | TYR | C | 249 | −16.570 | 52.523 | −16.435 | 1.00 | 41.98 | N |
| ATOM | 7818 | CA | TYR | C | 249 | −17.735 | 51.785 | −15.963 | 1.00 | 37.93 | C |
| ATOM | 7820 | CB | TYR | C | 249 | −18.109 | 52.235 | −14.549 | 1.00 | 41.16 | C |
| ATOM | 7823 | CG | TYR | C | 249 | −17.057 | 51.978 | −13.510 | 1.00 | 34.86 | C |
| ATOM | 7824 | CD1 | TYR | C | 249 | −17.009 | 50.771 | −12.841 | 1.00 | 33.81 | C |
| ATOM | 7826 | CE1 | TYR | C | 249 | −16.066 | 50.539 | −11.863 | 1.00 | 34.28 | C |
| ATOM | 7828 | CZ | TYR | C | 249 | −15.143 | 51.513 | −11.562 | 1.00 | 38.82 | C |
| ATOM | 7829 | OH | TYR | C | 249 | −14.163 | 51.233 | −10.645 | 1.00 | 38.08 | O |
| ATOM | 7831 | CE2 | TYR | C | 249 | −15.176 | 52.730 | −12.204 | 1.00 | 38.66 | C |
| ATOM | 7833 | CD2 | TYR | C | 249 | −16.153 | 52.960 | −13.157 | 1.00 | 34.49 | C |
| ATOM | 7835 | C | TYR | C | 249 | −18.864 | 52.130 | −16.926 | 1.00 | 42.56 | C |
| ATOM | 7836 | O | TYR | C | 249 | −19.101 | 53.296 | −17.256 | 1.00 | 45.45 | O |
| ATOM | 7837 | N | TYR | C | 250 | −19.534 | 51.106 | −17.420 | 1.00 | 39.02 | N |
| ATOM | 7839 | CA | TYR | C | 250 | −20.716 | 51.329 | −18.216 | 1.00 | 38.08 | C |
| ATOM | 7841 | CB | TYR | C | 250 | −20.638 | 50.482 | −19.478 | 1.00 | 36.98 | C |
| ATOM | 7844 | CG | TYR | C | 250 | −19.541 | 50.901 | −20.436 | 1.00 | 34.14 | C |
| ATOM | 7845 | CD1 | TYR | C | 250 | −18.214 | 50.548 | −20.218 | 1.00 | 28.53 | C |
| ATOM | 7847 | CE1 | TYR | C | 250 | −17.222 | 50.837 | −21.157 | 1.00 | 40.36 | C |
| ATOM | 7849 | CZ | TYR | C | 250 | −17.554 | 51.508 | −22.298 | 1.00 | 39.77 | C |
| ATOM | 7850 | OH | TYR | C | 250 | −16.552 | 51.865 | −23.139 | 1.00 | 42.08 | O |
| ATOM | 7852 | CE2 | TYR | C | 250 | −18.839 | 51.904 | −22.521 | 1.00 | 33.42 | C |
| ATOM | 7854 | CD2 | TYR | C | 250 | −19.839 | 51.552 | −21.621 | 1.00 | 21.46 | C |
| ATOM | 7856 | C | TYR | C | 250 | −21.953 | 50.969 | −17.386 | 1.00 | 42.37 | C |
| ATOM | 7857 | O | TYR | C | 250 | −21.957 | 50.002 | −16.623 | 1.00 | 43.51 | O |
| ATOM | 7858 | N | LEU | C | 251 | −22.998 | 51.778 | −17.515 | 1.00 | 46.56 | N |
| ATOM | 7860 | CA | LEU | C | 251 | −24.204 | 51.599 | −16.724 | 1.00 | 43.40 | C |
| ATOM | 7862 | CB | LEU | C | 251 | −24.255 | 52.622 | −15.598 | 1.00 | 41.08 | C |
| ATOM | 7865 | CG | LEU | C | 251 | −25.478 | 52.401 | −14.716 | 1.00 | 40.88 | C |
| ATOM | 7867 | CD1 | LEU | C | 251 | −25.591 | 50.958 | −14.238 | 1.00 | 38.41 | C |
| ATOM | 7871 | CD2 | LEU | C | 251 | −25.477 | 53.344 | −13.566 | 1.00 | 38.42 | C |
| ATOM | 7875 | C | LEU | C | 251 | −25.437 | 51.745 | −17.604 | 1.00 | 45.03 | C |
| ATOM | 7876 | O | LEU | C | 251 | −25.699 | 52.793 | −18.213 | 1.00 | 46.25 | O |
| ATOM | 7877 | N | ALA | C | 252 | −26.194 | 50.659 | −17.680 | 1.00 | 42.51 | N |
| ATOM | 7879 | CA | ALA | C | 252 | −27.405 | 50.655 | −18.488 | 1.00 | 43.50 | C |
| ATOM | 7881 | CB | ALA | C | 252 | −27.946 | 49.203 | −18.619 | 1.00 | 39.42 | C |
| ATOM | 7885 | C | ALA | C | 252 | −28.464 | 51.599 | −17.891 | 1.00 | 41.28 | C |
| ATOM | 7886 | O | ALA | C | 252 | −28.709 | 51.591 | −16.680 | 1.00 | 40.28 | O |
| ATOM | 7887 | N | PRO | C | 253 | −29.152 | 52.362 | −18.737 | 1.00 | 43.37 | N |
| ATOM | 7888 | CA | PRO | C | 253 | −30.183 | 53.292 | −18.251 | 1.00 | 45.47 | C |
| ATOM | 7890 | CB | PRO | C | 253 | −30.588 | 54.111 | −19.470 | 1.00 | 44.79 | C |
| ATOM | 7893 | CG | PRO | C | 253 | −30.173 | 53.296 | −20.625 | 1.00 | 54.38 | C |
| ATOM | 7896 | CD | PRO | C | 253 | −29.042 | 52.372 | −20.202 | 1.00 | 48.62 | C |
| ATOM | 7899 | C | PRO | C | 253 | −31.379 | 52.552 | −17.740 | 1.00 | 44.29 | C |
| ATOM | 7900 | O | PRO | C | 253 | −31.500 | 51.357 | −17.985 | 1.00 | 44.24 | O |
| ATOM | 7901 | N | LYS | C | 254 | −32.194 | 53.254 | −16.965 | 1.00 | 42.83 | N |
| ATOM | 7903 | CA | LYS | C | 254 | −33.605 | 52.940 | −16.855 | 1.00 | 44.02 | C |
| ATOM | 7905 | CB | LYS | C | 254 | −34.086 | 53.305 | −15.468 | 1.00 | 43.95 | C |
| ATOM | 7908 | CG | LYS | C | 254 | −33.344 | 52.670 | −14.309 | 1.00 | 25.78 | C |
| ATOM | 7911 | CD | LYS | C | 254 | −34.249 | 52.647 | −13.105 | 1.00 | 41.66 | C |
| ATOM | 7914 | CE | LYS | C | 254 | −33.662 | 51.981 | −11.880 | 1.00 | 55.76 | C |
| ATOM | 7917 | NZ | LYS | C | 254 | −34.496 | 52.261 | −10.653 | 1.00 | 56.69 | N |
| ATOM | 7921 | C | LYS | C | 254 | −34.421 | 53.697 | −17.906 | 1.00 | 53.68 | C |
| ATOM | 7922 | O | LYS | C | 254 | −34.371 | 54.921 | −17.988 | 1.00 | 58.11 | O |
| ATOM | 7923 | N | ILE | C | 255 | −35.134 | 52.948 | −18.739 | 1.00 | 65.17 | N |
| ATOM | 7925 | CA | ILE | C | 255 | −35.934 | 53.517 | −19.814 | 1.00 | 70.75 | C |
| ATOM | 7927 | CB | ILE | C | 255 | −35.841 | 52.628 | −21.059 | 1.00 | 71.87 | C |
| ATOM | 7929 | CG1 | ILE | C | 255 | −34.375 | 52.525 | −21.464 | 1.00 | 78.03 | C |
| ATOM | 7932 | CD1 | ILE | C | 255 | −33.799 | 53.822 | −21.996 | 1.00 | 74.63 | C |
| ATOM | 7936 | CG2 | ILE | C | 255 | −36.662 | 53.210 | −22.214 | 1.00 | 77.62 | C |
| ATOM | 7940 | C | ILE | C | 255 | −37.371 | 53.611 | −19.338 | 1.00 | 76.12 | C |
| ATOM | 7941 | O | ILE | C | 255 | −37.775 | 52.853 | −18.449 | 1.00 | 70.01 | O |
| ATOM | 7942 | N | GLU | C | 256 | −38.107 | 54.568 | −19.904 | 1.00 | 87.09 | N |
| ATOM | 7944 | CA | GLU | C | 256 | −39.502 | 54.791 | −19.540 | 1.00 | 94.29 | C |
| ATOM | 7946 | CB | GLU | C | 256 | −40.000 | 56.194 | −19.940 | 1.00 | 98.59 | C |
| ATOM | 7949 | CG | GLU | C | 256 | −41.484 | 56.473 | −19.660 | 1.00 | 107.03 | C |
| ATOM | 7952 | CD | GLU | C | 256 | −41.808 | 56.866 | −18.215 | 1.00 | 101.21 | C |
| ATOM | 7953 | OE1 | GLU | C | 256 | −41.571 | 58.034 | −17.847 | 1.00 | 98.40 | O |
| ATOM | 7954 | OE2 | GLU | C | 256 | −42.323 | 56.028 | −17.439 | 1.00 | 88.41 | O |
| ATOM | 7955 | C | GLU | C | 256 | −40.328 | 53.713 | −20.200 | 1.00 | 95.29 | C |
| ATOM | 7956 | O | GLU | C | 256 | −40.060 | 53.340 | −21.344 | 1.00 | 89.36 | O |
| ATOM | 7957 | N | ASP | C | 257 | −41.355 | 53.279 | −19.468 | 1.00 | 106.55 | N |
| ATOM | 7959 | CA | ASP | C | 257 | −41.669 | 51.864 | −19.196 | 1.00 | 114.78 | C |
| ATOM | 7961 | CB | ASP | C | 257 | −43.162 | 51.589 | −19.474 | 1.00 | 115.64 | C |
| ATOM | 7964 | CG | ASP | C | 257 | −43.417 | 50.207 | −20.055 | 1.00 | 118.07 | C |
| ATOM | 7965 | OD1 | ASP | C | 257 | −42.682 | 49.249 | −19.716 | 1.00 | 107.59 | O |
| ATOM | 7966 | OD2 | ASP | C | 257 | −44.356 | 49.997 | −20.854 | 1.00 | 122.55 | O |
| ATOM | 7967 | C | ASP | C | 257 | −40.767 | 50.862 | −19.915 | 1.00 | 115.43 | C |
| ATOM | 7968 | O | ASP | C | 257 | −41.009 | 50.511 | −21.068 | 1.00 | 119.12 | O |
| ATOM | 7969 | N | MET | E | 1 | −0.789 | 48.580 | 53.594 | 1.00 | 69.74 | N |
| ATOM | 7971 | CA | MET | E | 1 | −1.328 | 49.608 | 52.654 | 1.00 | 69.47 | C |
| ATOM | 7973 | CB | MET | E | 1 | −1.301 | 51.004 | 53.281 | 1.00 | 70.69 | C |
| ATOM | 7976 | CG | MET | E | 1 | −1.515 | 52.114 | 52.268 | 1.00 | 79.35 | C |
| ATOM | 7979 | SD | MET | E | 1 | −3.194 | 52.854 | 52.435 | 1.00 | 115.81 | S |
| ATOM | 7980 | CE | MET | E | 1 | −4.243 | 51.887 | 51.401 | 1.00 | 93.93 | C |
| ATOM | 7984 | C | MET | E | 1 | −0.482 | 49.618 | 51.384 | 1.00 | 65.11 | C |
| ATOM | 7985 | O | MET | E | 1 | 0.742 | 49.743 | 51.446 | 1.00 | 65.62 | O |
| ATOM | 7988 | N | PHE | E | 2 | −1.153 | 49.489 | 50.244 | 1.00 | 56.31 | N |
| ATOM | 7990 | CA | PHE | E | 2 | −0.553 | 49.702 | 48.931 | 1.00 | 48.35 | C |
| ATOM | 7992 | CB | PHE | E | 2 | −1.064 | 48.600 | 48.023 | 1.00 | 46.88 | C |
| ATOM | 7995 | CG | PHE | E | 2 | −0.713 | 48.778 | 46.590 | 1.00 | 30.79 | C |
| ATOM | 7996 | CD1 | PHE | E | 2 | 0.578 | 48.998 | 46.208 | 1.00 | 42.53 | C |
| ATOM | 7998 | CE1 | PHE | E | 2 | 0.902 | 49.089 | 44.883 | 1.00 | 38.02 | C |
| ATOM | 8000 | CZ | PHE | E | 2 | −0.075 | 49.007 | 43.935 | 1.00 | 34.49 | C |
| ATOM | 8002 | CE2 | PHE | E | 2 | −1.358 | 48.702 | 44.309 | 1.00 | 39.18 | C |
| ATOM | 8004 | CD2 | PHE | E | 2 | −1.666 | 48.592 | 45.623 | 1.00 | 40.04 | C |
| ATOM | 8006 | C | PHE | E | 2 | −0.923 | 51.054 | 48.312 | 1.00 | 50.09 | C |
| ATOM | 8007 | O | PHE | E | 2 | −2.100 | 51.414 | 48.138 | 1.00 | 43.58 | O |
| ATOM | 8008 | N | GLU | E | 3 | 0.098 | 51.803 | 47.931 | 1.00 | 44.85 | N |
| ATOM | 8010 | CA | GLU | E | 3 | −0.141 | 52.854 | 46.964 | 1.00 | 49.28 | C |
| ATOM | 8012 | CB | GLU | E | 3 | −0.515 | 54.165 | 47.677 | 1.00 | 46.70 | C |
| ATOM | 8015 | CG | GLU | E | 3 | 0.048 | 55.403 | 47.020 | 1.00 | 57.00 | C |
| ATOM | 8018 | CD | GLU | E | 3 | −0.356 | 56.658 | 47.741 | 1.00 | 67.19 | C |
| ATOM | 8019 | OE1 | GLU | E | 3 | −1.192 | 56.505 | 48.660 | 1.00 | 63.35 | O |
| ATOM | 8020 | OE2 | GLU | E | 3 | 0.177 | 57.742 | 47.384 | 1.00 | 59.34 | O |
| ATOM | 8021 | C | GLU | E | 3 | 0.962 | 53.005 | 45.903 | 1.00 | 45.29 | C |
| ATOM | 8022 | O | GLU | E | 3 | 2.159 | 52.998 | 46.178 | 1.00 | 42.90 | O |
| ATOM | 8023 | N | ALA | E | 4 | 0.534 | 53.118 | 44.654 | 1.00 | 44.42 | N |
| ATOM | 8025 | CA | ALA | E | 4 | 1.434 | 53.507 | 43.590 | 1.00 | 41.89 | C |
| ATOM | 8027 | CB | ALA | E | 4 | 1.760 | 52.356 | 42.638 | 1.00 | 44.57 | C |
| ATOM | 8031 | C | ALA | E | 4 | 0.769 | 54.561 | 42.802 | 1.00 | 44.19 | C |
| ATOM | 8032 | O | ALA | E | 4 | −0.450 | 54.565 | 42.592 | 1.00 | 40.96 | O |
| ATOM | 8033 | N | ARG | E | 5 | 1.660 | 55.327 | 42.206 | 1.00 | 47.35 | N |
| ATOM | 8035 | CA | ARG | E | 5 | 1.258 | 56.457 | 41.414 | 1.00 | 51.59 | C |
| ATOM | 8037 | CB | ARG | E | 5 | 1.394 | 57.747 | 42.245 | 1.00 | 56.75 | C |
| ATOM | 8040 | CG | ARG | E | 5 | 1.432 | 59.051 | 41.467 | 1.00 | 54.55 | C |
| ATOM | 8043 | CD | ARG | E | 5 | 1.592 | 60.224 | 42.383 | 1.00 | 66.51 | C |
| ATOM | 8046 | NE | ARG | E | 5 | 2.976 | 60.688 | 42.432 | 1.00 | 69.66 | N |
| ATOM | 8048 | CZ | ARG | E | 5 | 3.341 | 61.894 | 42.030 | 1.00 | 65.45 | C |
| ATOM | 8049 | NH1 | ARG | E | 5 | 2.416 | 62.719 | 41.555 | 1.00 | 81.01 | N |
| ATOM | 8052 | NH2 | ARG | E | 5 | 4.608 | 62.283 | 42.109 | 1.00 | 62.71 | N |
| ATOM | 8055 | C | ARG | E | 5 | 2.090 | 56.463 | 40.144 | 1.00 | 42.71 | C |
| ATOM | 8056 | O | ARG | E | 5 | 3.272 | 56.163 | 40.135 | 1.00 | 37.31 | O |
| ATOM | 8057 | N | LEU | E | 6 | 1.422 | 56.779 | 39.051 | 1.00 | 42.89 | N |
| ATOM | 8059 | CA | LEU | E | 6 | 2.065 | 56.832 | 37.755 | 1.00 | 38.85 | C |
| ATOM | 8061 | CB | LEU | E | 6 | 1.631 | 55.607 | 36.973 | 1.00 | 27.51 | C |
| ATOM | 8064 | CG | LEU | E | 6 | 2.522 | 55.126 | 35.854 | 1.00 | 39.66 | C |
| ATOM | 8066 | CD1 | LEU | E | 6 | 3.976 | 54.918 | 36.294 | 1.00 | 60.35 | C |
| ATOM | 8070 | CD2 | LEU | E | 6 | 1.881 | 53.825 | 35.419 | 1.00 | 54.31 | C |
| ATOM | 8074 | C | LEU | E | 6 | 1.593 | 58.099 | 37.063 | 1.00 | 39.97 | C |
| ATOM | 8075 | O | LEU | E | 6 | 0.405 | 58.282 | 36.813 | 1.00 | 37.79 | O |
| ATOM | 8076 | N | VAL | E | 7 | 2.520 | 59.002 | 36.790 | 1.00 | 44.83 | N |
| ATOM | 8078 | CA | VAL | E | 7 | 2.151 | 60.265 | 36.153 | 1.00 | 49.23 | C |
| ATOM | 8080 | CB | VAL | E | 7 | 3.287 | 61.289 | 36.292 | 1.00 | 53.19 | C |
| ATOM | 8082 | CG1 | VAL | E | 7 | 2.914 | 62.612 | 35.642 | 1.00 | 49.40 | C |
| ATOM | 8086 | CG2 | VAL | E | 7 | 3.630 | 61.441 | 37.764 | 1.00 | 50.82 | C |
| ATOM | 8090 | C | VAL | E | 7 | 1.839 | 60.065 | 34.671 | 1.00 | 49.16 | C |
| ATOM | 8091 | O | VAL | E | 7 | 0.954 | 60.732 | 34.140 | 1.00 | 45.76 | O |
| ATOM | 8092 | N | GLN | E | 8 | 2.566 | 59.160 | 34.015 | 1.00 | 46.81 | N |
| ATOM | 8094 | CA | GLN | E | 8 | 2.209 | 58.778 | 32.671 | 1.00 | 46.64 | C |
| ATOM | 8096 | CB | GLN | E | 8 | 3.372 | 58.174 | 31.876 | 1.00 | 48.22 | C |
| ATOM | 8099 | CG | GLN | E | 8 | 4.333 | 59.198 | 31.245 | 1.00 | 65.12 | C |
| ATOM | 8102 | CD | GLN | E | 8 | 5.356 | 58.559 | 30.304 | 1.00 | 64.23 | C |
| ATOM | 8103 | OE1 | GLN | E | 8 | 4.996 | 58.106 | 29.231 | 1.00 | 62.18 | O |
| ATOM | 8104 | NE2 | GLN | E | 8 | 6.620 | 58.519 | 30.706 | 1.00 | 79.33 | N |
| ATOM | 8107 | C | GLN | E | 8 | 1.108 | 57.765 | 32.859 | 1.00 | 47.14 | C |
| ATOM | 8108 | O | GLN | E | 8 | 1.339 | 56.564 | 32.752 | 1.00 | 52.64 | O |
| ATOM | 8109 | N | GLY | E | 9 | −0.095 | 58.278 | 33.084 | 1.00 | 41.57 | N |
| ATOM | 8111 | CA | GLY | E | 9 | −1.259 | 57.456 | 33.307 | 1.00 | 42.46 | C |
| ATOM | 8114 | C | GLY | E | 9 | −1.485 | 56.525 | 32.140 | 1.00 | 46.61 | C |
| ATOM | 8115 | O | GLY | E | 9 | −2.280 | 55.576 | 32.251 | 1.00 | 43.42 | O |
| ATOM | 8116 | N | SER | E | 10 | −0.866 | 56.849 | 31.003 | 1.00 | 48.24 | N |
| ATOM | 8118 | CA | SER | E | 10 | −1.317 | 56.280 | 29.719 | 1.00 | 51.07 | C |
| ATOM | 8120 | CB | SER | E | 10 | −0.824 | 57.136 | 28.545 | 1.00 | 55.17 | C |
| ATOM | 8123 | OG | SER | E | 10 | 0.596 | 57.249 | 28.531 | 1.00 | 48.81 | O |
| ATOM | 8125 | C | SER | E | 10 | −0.739 | 54.864 | 29.629 | 1.00 | 49.45 | C |
| ATOM | 8126 | O | SER | E | 10 | −1.237 | 53.979 | 28.938 | 1.00 | 34.32 | O |
| ATOM | 8127 | N | ILE | E | 11 | 0.322 | 54.669 | 30.403 | 1.00 | 47.33 | N |
| ATOM | 8129 | CA | ILE | E | 11 | 1.006 | 53.401 | 30.437 | 1.00 | 40.71 | C |
| ATOM | 8131 | CB | ILE | E | 11 | 2.144 | 53.451 | 31.468 | 1.00 | 46.68 | C |
| ATOM | 8133 | CG1 | ILE | E | 11 | 3.290 | 54.332 | 30.971 | 1.00 | 42.40 | C |
| ATOM | 8136 | CD1 | ILE | E | 11 | 4.464 | 54.295 | 31.908 | 1.00 | 50.73 | C |
| ATOM | 8140 | CG2 | ILE | E | 11 | 2.604 | 52.043 | 31.811 | 1.00 | 28.27 | C |
| ATOM | 8144 | C | ILE | E | 11 | −0.004 | 52.406 | 30.918 | 1.00 | 39.53 | C |
| ATOM | 8145 | O | ILE | E | 11 | 0.040 | 51.256 | 30.537 | 1.00 | 48.50 | O |
| ATOM | 8146 | N | LEU | E | 12 | −0.820 | 52.805 | 31.879 | 1.00 | 40.83 | N |
| ATOM | 8148 | CA | LEU | E | 12 | −1.575 | 51.812 | 32.599 | 1.00 | 38.15 | C |
| ATOM | 8150 | CB | LEU | E | 12 | −1.914 | 52.281 | 34.003 | 1.00 | 41.91 | C |
| ATOM | 8153 | CG | LEU | E | 12 | −2.713 | 51.244 | 34.779 | 1.00 | 52.75 | C |
| ATOM | 8155 | CD1 | LEU | E | 12 | −1.880 | 50.016 | 35.136 | 1.00 | 54.59 | C |
| ATOM | 8159 | CD2 | LEU | E | 12 | −3.291 | 51.915 | 36.004 | 1.00 | 59.38 | C |
| ATOM | 8163 | C | LEU | E | 12 | −2.790 | 51.537 | 31.757 | 1.00 | 37.99 | C |
| ATOM | 8164 | O | LEU | E | 12 | −3.210 | 50.378 | 31.608 | 1.00 | 40.11 | O |
| ATOM | 8165 | N | LYS | E | 13 | −3.238 | 52.577 | 31.062 | 1.00 | 42.73 | N |
| ATOM | 8167 | CA | LYS | E | 13 | −4.259 | 52.391 | 30.015 | 1.00 | 44.16 | C |
| ATOM | 8169 | CB | LYS | E | 13 | −4.720 | 53.709 | 29.384 | 1.00 | 37.83 | C |
| ATOM | 8172 | CG | LYS | E | 13 | −5.406 | 54.687 | 30.340 | 1.00 | 41.50 | C |
| ATOM | 8175 | CD | LYS | E | 13 | −5.474 | 56.136 | 29.816 | 1.00 | 53.18 | C |
| ATOM | 8178 | CE | LYS | E | 13 | −6.507 | 56.378 | 28.699 | 1.00 | 30.25 | C |
| ATOM | 8181 | NZ | LYS | E | 13 | −7.821 | 55.694 | 29.029 | 1.00 | 59.73 | N |
| ATOM | 8185 | C | LYS | E | 13 | −3.857 | 51.400 | 28.927 | 1.00 | 36.23 | C |
| ATOM | 8186 | O | LYS | E | 13 | −4.515 | 50.372 | 28.734 | 1.00 | 42.28 | O |
| ATOM | 8187 | N | LYS | E | 14 | −2.754 | 51.677 | 28.261 | 1.00 | 29.38 | N |
| ATOM | 8189 | CA | LYS | E | 14 | −2.212 | 50.756 | 27.276 | 1.00 | 32.57 | C |
| ATOM | 8191 | CB | LYS | E | 14 | −0.880 | 51.313 | 26.799 | 1.00 | 37.89 | C |
| ATOM | 8194 | CG | LYS | E | 14 | −1.003 | 52.707 | 26.173 | 1.00 | 38.20 | C |
| ATOM | 8197 | CD | LYS | E | 14 | 0.339 | 53.385 | 25.812 | 1.00 | 35.60 | C |
| ATOM | 8200 | CE | LYS | E | 14 | 0.132 | 54.481 | 24.792 | 1.00 | 28.92 | C |
| ATOM | 8203 | NZ | LYS | E | 14 | 1.321 | 55.190 | 24.351 | 1.00 | 45.87 | N |
| ATOM | 8207 | C | LYS | E | 14 | −2.016 | 49.299 | 27.743 | 1.00 | 39.31 | C |
| ATOM | 8208 | O | LYS | E | 14 | −2.266 | 48.351 | 26.968 | 1.00 | 40.32 | O |
| ATOM | 8209 | N | VAL | E | 15 | −1.481 | 49.101 | 28.951 | 1.00 | 40.28 | N |
| ATOM | 8211 | CA | VAL | E | 15 | −1.239 | 47.744 | 29.474 | 1.00 | 34.97 | C |
| ATOM | 8213 | CB | VAL | E | 15 | −0.518 | 47.771 | 30.821 | 1.00 | 33.47 | C |
| ATOM | 8215 | CG1 | VAL | E | 15 | −0.753 | 46.491 | 31.617 | 1.00 | 30.71 | C |
| ATOM | 8219 | CG2 | VAL | E | 15 | 0.960 | 48.025 | 30.628 | 1.00 | 34.25 | C |
| ATOM | 8223 | C | VAL | E | 15 | −2.566 | 47.027 | 29.663 | 1.00 | 37.24 | C |
| ATOM | 8224 | O | VAL | E | 15 | −2.769 | 45.870 | 29.276 | 1.00 | 26.15 | O |
| ATOM | 8225 | N | LEU | E | 16 | −3.544 | 47.744 | 30.191 | 1.00 | 46.23 | N |
| ATOM | 8227 | CA | LEU | E | 16 | −4.852 | 47.088 | 30.306 | 1.00 | 47.43 | C |
| ATOM | 8229 | CB | LEU | E | 16 | −5.868 | 47.858 | 31.151 | 1.00 | 42.71 | C |
| ATOM | 8232 | CG | LEU | E | 16 | −5.781 | 46.949 | 32.364 | 1.00 | 45.09 | C |
| ATOM | 8234 | CD1 | LEU | E | 16 | −5.475 | 47.750 | 33.594 | 1.00 | 55.30 | C |
| ATOM | 8238 | CD2 | LEU | E | 16 | −7.004 | 46.107 | 32.465 | 1.00 | 39.05 | C |
| ATOM | 8242 | C | LEU | E | 16 | −5.419 | 46.719 | 28.971 | 1.00 | 32.76 | C |
| ATOM | 8243 | O | LEU | E | 16 | −5.987 | 45.662 | 28.794 | 1.00 | 43.45 | O |
| ATOM | 8244 | N | GLU | E | 17 | −5.248 | 47.615 | 28.020 | 1.00 | 42.64 | N |
| ATOM | 8246 | CA | GLU | E | 17 | −5.814 | 47.449 | 26.681 | 1.00 | 42.20 | C |
| ATOM | 8248 | CB | GLU | E | 17 | −5.614 | 48.765 | 25.904 | 1.00 | 31.04 | C |
| ATOM | 8251 | CG | GLU | E | 17 | −6.624 | 49.043 | 24.814 | 1.00 | 63.06 | C |
| ATOM | 8254 | CD | GLU | E | 17 | −7.942 | 49.550 | 25.329 | 1.00 | 72.21 | C |
| ATOM | 8255 | OE1 | GLU | E | 17 | −8.509 | 48.974 | 26.314 | 1.00 | 71.04 | O |
| ATOM | 8256 | OE2 | GLU | E | 17 | −8.365 | 50.541 | 24.694 | 1.00 | 78.32 | O |
| ATOM | 8257 | C | GLU | E | 17 | −5.073 | 46.269 | 26.061 | 1.00 | 37.19 | C |
| ATOM | 8258 | O | GLU | E | 17 | −5.586 | 45.517 | 25.233 | 1.00 | 44.05 | O |
| ATOM | 8259 | N | ALA | E | 18 | −3.866 | 46.048 | 26.567 | 1.00 | 43.43 | N |
| ATOM | 8261 | CA | ALA | E | 18 | −2.997 | 45.060 | 25.975 | 1.00 | 41.41 | C |
| ATOM | 8263 | CB | ALA | E | 18 | −1.585 | 45.371 | 26.310 | 1.00 | 44.85 | C |
| ATOM | 8267 | C | ALA | E | 18 | −3.379 | 43.698 | 26.479 | 1.00 | 40.09 | C |
| ATOM | 8268 | O | ALA | E | 18 | −2.983 | 42.712 | 25.907 | 1.00 | 53.77 | O |
| ATOM | 8269 | N | LEU | E | 19 | −4.153 | 43.652 | 27.554 | 1.00 | 46.96 | N |
| ATOM | 8271 | CA | LEU | E | 19 | −4.427 | 42.407 | 28.258 | 1.00 | 41.48 | C |
| ATOM | 8273 | CB | LEU | E | 19 | −4.227 | 42.593 | 29.757 | 1.00 | 36.88 | C |
| ATOM | 8276 | CG | LEU | E | 19 | −2.772 | 42.668 | 30.186 | 1.00 | 47.84 | C |
| ATOM | 8278 | CD1 | LEU | E | 19 | −2.825 | 42.966 | 31.661 | 1.00 | 49.12 | C |
| ATOM | 8282 | CD2 | LEU | E | 19 | −2.025 | 41.363 | 29.891 | 1.00 | 51.89 | C |
| ATOM | 8286 | C | LEU | E | 19 | −5.860 | 41.951 | 28.020 | 1.00 | 50.21 | C |
| ATOM | 8287 | O | LEU | E | 19 | −6.105 | 40.713 | 27.983 | 1.00 | 36.28 | O |
| ATOM | 8288 | N | LYS | E | 20 | −6.776 | 42.937 | 27.933 | 1.00 | 45.89 | N |
| ATOM | 8290 | CA | LYS | E | 20 | −8.201 | 42.668 | 28.123 | 1.00 | 48.93 | C |
| ATOM | 8292 | CB | LYS | E | 20 | −9.099 | 43.911 | 28.310 | 1.00 | 51.38 | C |
| ATOM | 8295 | CG | LYS | E | 20 | −9.047 | 45.021 | 27.289 | 1.00 | 57.99 | C |
| ATOM | 8298 | CD | LYS | E | 20 | −10.185 | 45.016 | 26.248 | 1.00 | 64.15 | C |
| ATOM | 8301 | CE | LYS | E | 20 | −11.084 | 46.271 | 26.342 | 1.00 | 80.82 | C |
| ATOM | 8304 | NZ | LYS | E | 20 | −10.950 | 47.269 | 25.243 | 1.00 | 76.02 | N |
| ATOM | 8308 | C | LYS | E | 20 | −8.732 | 41.763 | 27.032 | 1.00 | 44.66 | C |
| ATOM | 8309 | O | LYS | E | 20 | −9.646 | 40.986 | 27.250 | 1.00 | 51.65 | O |
| ATOM | 8310 | N | ASP | E | 21 | −8.072 | 41.752 | 25.889 | 1.00 | 49.46 | N |
| ATOM | 8312 | CA | ASP | E | 21 | −8.589 | 40.961 | 24.787 | 1.00 | 55.88 | C |
| ATOM | 8314 | CB | ASP | E | 21 | −8.010 | 41.516 | 23.478 | 1.00 | 67.08 | C |
| ATOM | 8317 | CG | ASP | E | 21 | −8.876 | 42.608 | 22.842 | 1.00 | 77.80 | C |
| ATOM | 8318 | OD1 | ASP | E | 21 | −9.912 | 43.001 | 23.449 | 1.00 | 64.74 | O |
| ATOM | 8319 | OD2 | ASP | E | 21 | −8.552 | 43.108 | 21.726 | 1.00 | 75.94 | O |
| ATOM | 8320 | C | ASP | E | 21 | −8.148 | 39.508 | 24.975 | 1.00 | 53.34 | C |
| ATOM | 8321 | O | ASP | E | 21 | −8.799 | 38.589 | 24.505 | 1.00 | 54.00 | O |
| ATOM | 8322 | N | LEU | E | 22 | −6.989 | 39.302 | 25.596 | 1.00 | 51.28 | N |
| ATOM | 8324 | CA | LEU | E | 22 | −6.336 | 37.999 | 25.575 | 1.00 | 39.99 | C |
| ATOM | 8326 | CB | LEU | E | 22 | −4.832 | 38.147 | 25.695 | 1.00 | 32.69 | C |
| ATOM | 8329 | CG | LEU | E | 22 | −4.052 | 36.833 | 25.718 | 1.00 | 34.05 | C |
| ATOM | 8331 | CD1 | LEU | E | 22 | −4.150 | 36.098 | 24.387 | 1.00 | 34.00 | C |
| ATOM | 8335 | CD2 | LEU | E | 22 | −2.581 | 37.070 | 26.004 | 1.00 | 35.42 | C |
| ATOM | 8339 | C | LEU | E | 22 | −6.818 | 37.192 | 26.753 | 1.00 | 37.62 | C |
| ATOM | 8340 | O | LEU | E | 22 | −6.826 | 35.972 | 26.717 | 1.00 | 47.60 | O |
| ATOM | 8341 | N | ILE | E | 23 | −7.116 | 37.878 | 27.842 | 1.00 | 40.37 | N |
| ATOM | 8343 | CA | ILE | E | 23 | −7.476 | 37.210 | 29.080 | 1.00 | 46.36 | C |
| ATOM | 8345 | CB | ILE | E | 23 | −6.275 | 36.866 | 30.027 | 1.00 | 52.95 | C |
| ATOM | 8347 | CG1 | ILE | E | 23 | −5.647 | 38.111 | 30.614 | 1.00 | 58.59 | C |
| ATOM | 8350 | CD1 | ILE | E | 23 | −5.165 | 37.834 | 31.993 | 1.00 | 79.34 | C |
| ATOM | 8354 | CG2 | ILE | E | 23 | −5.134 | 36.083 | 29.371 | 1.00 | 69.23 | C |
| ATOM | 8358 | C | ILE | E | 23 | −8.401 | 38.107 | 29.866 | 1.00 | 46.32 | C |
| ATOM | 8359 | O | ILE | E | 23 | −8.449 | 39.316 | 29.695 | 1.00 | 43.65 | O |
| ATOM | 8360 | N | ASN | E | 24 | −9.057 | 37.469 | 30.814 | 1.00 | 52.56 | N |
| ATOM | 8362 | CA | ASN | E | 24 | −10.442 | 37.704 | 31.142 | 1.00 | 52.62 | C |
| ATOM | 8364 | CB | ASN | E | 24 | −11.262 | 36.386 | 30.946 | 1.00 | 53.52 | C |
| ATOM | 8367 | CG | ASN | E | 24 | −10.432 | 35.016 | 31.158 | 1.00 | 70.26 | C |
| ATOM | 8368 | OD1 | ASN | E | 24 | −9.468 | 34.686 | 30.466 | 1.00 | 73.61 | O |
| ATOM | 8369 | ND2 | ASN | E | 24 | −10.936 | 34.171 | 32.049 | 1.00 | 84.35 | N |
| ATOM | 8372 | C | ASN | E | 24 | −10.274 | 38.266 | 32.579 | 1.00 | 52.48 | C |
| ATOM | 8373 | O | ASN | E | 24 | −10.397 | 39.474 | 32.817 | 1.00 | 48.41 | O |
| ATOM | 8374 | N | GLU | E | 25 | −9.767 | 37.420 | 33.475 | 1.00 | 48.60 | N |
| ATOM | 8376 | CA | GLU | E | 25 | −9.332 | 37.012 | 34.805 | 1.00 | 43.90 | C |
| ATOM | 8378 | CB | GLU | E | 25 | −10.267 | 37.204 | 35.847 | 1.00 | 44.01 | C |
| ATOM | 8381 | CG | GLU | E | 25 | −10.628 | 35.728 | 35.658 | 1.00 | 66.57 | C |
| ATOM | 8384 | CD | GLU | E | 25 | −12.077 | 35.375 | 36.004 | 1.00 | 72.74 | C |
| ATOM | 8385 | OE1 | GLU | E | 25 | −12.270 | 34.408 | 36.759 | 1.00 | 84.73 | O |
| ATOM | 8386 | OE2 | GLU | E | 25 | −13.038 | 36.023 | 35.526 | 1.00 | 65.95 | O |
| ATOM | 8387 | C | GLU | E | 25 | −7.912 | 37.359 | 35.116 | 1.00 | 49.57 | C |
| ATOM | 8388 | O | GLU | E | 25 | −7.415 | 36.382 | 34.576 | 1.00 | 62.03 | O |
| ATOM | 8389 | N | ALA | E | 26 | −7.257 | 38.046 | 36.040 | 1.00 | 51.23 | N |
| ATOM | 8391 | CA | ALA | E | 26 | −5.914 | 37.677 | 36.467 | 1.00 | 43.47 | C |
| ATOM | 8393 | CB | ALA | E | 26 | −4.906 | 38.326 | 35.583 | 1.00 | 41.41 | C |
| ATOM | 8397 | C | ALA | E | 26 | −5.746 | 38.215 | 37.882 | 1.00 | 51.21 | C |
| ATOM | 8398 | O | ALA | E | 26 | −6.526 | 39.073 | 38.315 | 1.00 | 54.34 | O |
| ATOM | 8399 | N | CYS | E | 27 | −4.704 | 37.762 | 38.579 | 1.00 | 47.52 | N |
| ATOM | 8401 | CA | CYS | E | 27 | −4.394 | 38.337 | 39.875 | 1.00 | 43.55 | C |
| ATOM | 8403 | CB | CYS | E | 27 | −4.365 | 37.271 | 40.976 | 1.00 | 46.87 | C |
| ATOM | 8406 | SG | CYS | E | 27 | −2.918 | 37.290 | 42.051 | 1.00 | 49.19 | S |
| ATOM | 8407 | C | CYS | E | 27 | −3.122 | 39.166 | 39.804 | 1.00 | 38.18 | C |
| ATOM | 8408 | O | CYS | E | 27 | −2.063 | 38.731 | 39.366 | 1.00 | 46.17 | O |
| ATOM | 8409 | N | TRP | E | 28 | −3.265 | 40.422 | 40.175 | 1.00 | 37.32 | N |
| ATOM | 8411 | CA | TRP | E | 28 | −2.133 | 41.305 | 40.289 | 1.00 | 36.91 | C |
| ATOM | 8413 | CB | TRP | E | 28 | −2.686 | 42.738 | 40.344 | 1.00 | 37.75 | C |
| ATOM | 8416 | CG | TRP | E | 28 | −3.372 | 43.108 | 39.050 | 1.00 | 29.42 | C |
| ATOM | 8417 | CD1 | TRP | E | 28 | −4.532 | 42.587 | 38.555 | 1.00 | 46.92 | C |
| ATOM | 8419 | NE1 | TRP | E | 28 | −4.834 | 43.182 | 37.354 | 1.00 | 48.79 | N |
| ATOM | 8421 | CE2 | TRP | E | 28 | −3.851 | 44.078 | 37.038 | 1.00 | 33.64 | C |
| ATOM | 8422 | CD2 | TRP | E | 28 | −2.929 | 44.075 | 38.089 | 1.00 | 30.22 | C |
| ATOM | 8423 | CE3 | TRP | E | 28 | −1.863 | 44.971 | 38.039 | 1.00 | 41.69 | C |
| ATOM | 8425 | CZ3 | TRP | E | 28 | −1.763 | 45.802 | 36.998 | 1.00 | 40.77 | C |
| ATOM | 8427 | CH2 | TRP | E | 28 | −2.659 | 45.740 | 35.931 | 1.00 | 43.88 | C |
| ATOM | 8429 | CZ2 | TRP | E | 28 | −3.707 | 44.883 | 35.929 | 1.00 | 42.40 | C |
| ATOM | 8431 | C | TRP | E | 28 | −1.344 | 40.950 | 41.554 | 1.00 | 43.51 | C |
| ATOM | 8432 | O | TRP | E | 28 | −1.870 | 40.845 | 42.675 | 1.00 | 40.38 | O |
| ATOM | 8433 | N | ASP | E | 29 | −0.053 | 40.745 | 41.388 | 1.00 | 41.58 | N |
| ATOM | 8435 | CA | ASP | E | 29 | 0.745 | 40.414 | 42.548 | 1.00 | 41.14 | C |
| ATOM | 8437 | CB | ASP | E | 29 | 1.739 | 39.315 | 42.194 | 1.00 | 44.21 | C |
| ATOM | 8440 | CG | ASP | E | 29 | 1.066 | 37.982 | 41.944 | 1.00 | 51.91 | C |
| ATOM | 8441 | OD1 | ASP | E | 29 | 0.279 | 37.572 | 42.837 | 1.00 | 52.90 | O |
| ATOM | 8442 | OD2 | ASP | E | 29 | 1.285 | 37.293 | 40.908 | 1.00 | 72.16 | O |
| ATOM | 8443 | C | ASP | E | 29 | 1.484 | 41.635 | 43.012 | 1.00 | 39.08 | C |
| ATOM | 8444 | O | ASP | E | 29 | 2.508 | 41.991 | 42.440 | 1.00 | 49.64 | O |
| ATOM | 8445 | N | ILE | E | 30 | 1.004 | 42.289 | 44.059 | 1.00 | 42.12 | N |
| ATOM | 8447 | CA | ILE | E | 30 | 1.713 | 43.489 | 44.525 | 1.00 | 41.56 | C |
| ATOM | 8449 | CB | ILE | E | 30 | 0.682 | 44.491 | 45.027 | 1.00 | 44.77 | C |
| ATOM | 8451 | CG1 | ILE | E | 30 | −0.532 | 44.516 | 44.091 | 1.00 | 47.19 | C |
| ATOM | 8454 | CD1 | ILE | E | 30 | −0.366 | 45.468 | 42.937 | 1.00 | 43.54 | C |
| ATOM | 8458 | CG2 | ILE | E | 30 | 1.290 | 45.876 | 45.148 | 1.00 | 56.85 | C |
| ATOM | 8462 | C | ILE | E | 30 | 2.733 | 43.157 | 45.626 | 1.00 | 37.04 | C |
| ATOM | 8463 | O | ILE | E | 30 | 2.476 | 42.313 | 46.485 | 1.00 | 43.92 | O |
| ATOM | 8464 | N | SER | E | 31 | 3.927 | 43.719 | 45.531 | 1.00 | 32.26 | N |
| ATOM | 8466 | CA | SER | E | 31 | 4.978 | 43.540 | 46.540 | 1.00 | 36.99 | C |
| ATOM | 8468 | CB | SER | E | 31 | 5.930 | 42.435 | 46.131 | 1.00 | 41.35 | C |
| ATOM | 8471 | OG | SER | E | 31 | 6.396 | 42.671 | 44.813 | 1.00 | 45.43 | O |
| ATOM | 8473 | C | SER | E | 31 | 5.890 | 44.738 | 46.619 | 1.00 | 36.56 | C |
| ATOM | 8474 | O | SER | E | 31 | 5.820 | 45.635 | 45.794 | 1.00 | 27.13 | O |
| ATOM | 8475 | N | SER | E | 32 | 6.840 | 44.681 | 47.540 | 1.00 | 45.59 | N |
| ATOM | 8477 | CA | SER | E | 32 | 7.768 | 45.801 | 47.685 | 1.00 | 51.12 | C |
| ATOM | 8479 | CB | SER | E | 32 | 8.649 | 45.587 | 48.911 | 1.00 | 53.61 | C |
| ATOM | 8482 | OG | SER | E | 32 | 9.605 | 44.590 | 48.648 | 1.00 | 53.15 | O |
| ATOM | 8484 | C | SER | E | 32 | 8.644 | 45.952 | 46.446 | 1.00 | 51.16 | C |
| ATOM | 8485 | O | SER | E | 32 | 9.250 | 46.998 | 46.216 | 1.00 | 50.60 | O |
| ATOM | 8486 | N | SER | E | 33 | 8.714 | 44.891 | 45.649 | 1.00 | 54.35 | N |
| ATOM | 8488 | CA | SER | E | 33 | 9.574 | 44.874 | 44.464 | 1.00 | 53.77 | C |
| ATOM | 8490 | CB | SER | E | 33 | 10.135 | 43.464 | 44.229 | 1.00 | 52.00 | C |
| ATOM | 8493 | OG | SER | E | 33 | 9.398 | 42.508 | 44.964 | 1.00 | 58.02 | O |
| ATOM | 8495 | C | SER | E | 33 | 8.811 | 45.376 | 43.242 | 1.00 | 43.17 | C |
| ATOM | 8496 | O | SER | E | 33 | 9.398 | 45.737 | 42.245 | 1.00 | 43.50 | O |
| ATOM | 8497 | N | GLY | E | 34 | 7.487 | 45.422 | 43.344 | 1.00 | 47.24 | N |
| ATOM | 8499 | CA | GLY | E | 34 | 6.654 | 46.239 | 42.481 | 1.00 | 38.90 | C |
| ATOM | 8502 | C | GLY | E | 34 | 5.442 | 45.406 | 42.132 | 1.00 | 36.30 | C |
| ATOM | 8503 | O | GLY | E | 34 | 5.078 | 44.550 | 42.935 | 1.00 | 30.73 | O |
| ATOM | 8504 | N | VAL | E | 35 | 4.917 | 45.581 | 40.912 | 1.00 | 41.72 | N |
| ATOM | 8506 | CA | VAL | E | 35 | 3.643 | 45.012 | 40.462 | 1.00 | 42.87 | C |
| ATOM | 8508 | CB | VAL | E | 35 | 2.800 | 46.086 | 39.822 | 1.00 | 40.94 | C |
| ATOM | 8510 | CG1 | VAL | E | 35 | 1.570 | 45.472 | 39.211 | 1.00 | 47.56 | C |
| ATOM | 8514 | CG2 | VAL | E | 35 | 2.430 | 47.111 | 40.851 | 1.00 | 40.95 | C |
| ATOM | 8518 | C | VAL | E | 35 | 3.797 | 43.967 | 39.368 | 1.00 | 42.25 | C |
| ATOM | 8519 | O | VAL | E | 35 | 4.433 | 44.247 | 38.366 | 1.00 | 42.94 | O |
| ATOM | 8520 | N | ASN | E | 36 | 3.157 | 42.810 | 39.533 | 1.00 | 38.53 | N |
| ATOM | 8522 | CA | ASN | E | 36 | 3.387 | 41.675 | 38.654 | 1.00 | 40.08 | C |
| ATOM | 8524 | CB | ASN | E | 36 | 4.368 | 40.679 | 39.245 | 1.00 | 38.70 | C |
| ATOM | 8527 | CG | ASN | E | 36 | 5.783 | 41.087 | 38.946 | 1.00 | 51.80 | C |
| ATOM | 8528 | OD1 | ASN | E | 36 | 5.992 | 42.181 | 38.450 | 1.00 | 60.42 | O |
| ATOM | 8529 | ND2 | ASN | E | 36 | 6.752 | 40.248 | 39.249 | 1.00 | 67.02 | N |
| ATOM | 8532 | C | ASN | E | 36 | 2.112 | 40.972 | 38.331 | 1.00 | 39.91 | C |
| ATOM | 8533 | O | ASN | E | 36 | 1.180 | 41.034 | 39.120 | 1.00 | 47.36 | O |
| ATOM | 8534 | N | LEU | E | 37 | 2.057 | 40.386 | 37.135 | 1.00 | 36.71 | N |
| ATOM | 8536 | CA | LEU | E | 37 | 0.870 | 39.696 | 36.651 | 1.00 | 33.61 | C |
| ATOM | 8538 | CB | LEU | E | 37 | −0.047 | 40.633 | 35.866 | 1.00 | 34.89 | C |
| ATOM | 8541 | CG | LEU | E | 37 | −1.450 | 40.133 | 35.495 | 1.00 | 32.10 | C |
| ATOM | 8543 | CD1 | LEU | E | 37 | −2.566 | 41.148 | 35.700 | 1.00 | 47.31 | C |
| ATOM | 8547 | CD2 | LEU | E | 37 | −1.460 | 39.804 | 34.037 | 1.00 | 38.82 | C |
| ATOM | 8551 | C | LEU | E | 37 | 1.334 | 38.590 | 35.742 | 1.00 | 34.51 | C |
| ATOM | 8552 | O | LEU | E | 37 | 2.159 | 38.827 | 34.892 | 1.00 | 40.54 | O |
| ATOM | 8553 | N | GLN | E | 38 | 0.801 | 37.386 | 35.932 | 1.00 | 40.35 | N |
| ATOM | 8555 | CA | GLN | E | 38 | 1.008 | 36.260 | 35.022 | 1.00 | 37.77 | C |
| ATOM | 8557 | CB | GLN | E | 38 | 2.079 | 35.352 | 35.606 | 1.00 | 29.73 | C |
| ATOM | 8560 | CG | GLN | E | 38 | 2.462 | 34.226 | 34.711 | 1.00 | 45.68 | C |
| ATOM | 8563 | CD | GLN | E | 38 | 3.139 | 33.087 | 35.449 | 1.00 | 56.71 | C |
| ATOM | 8564 | OE1 | GLN | E | 38 | 2.483 | 32.124 | 35.865 | 1.00 | 63.02 | O |
| ATOM | 8565 | NE2 | GLN | E | 38 | 4.459 | 33.141 | 35.513 | 1.00 | 27.75 | N |
| ATOM | 8568 | C | GLN | E | 38 | −0.278 | 35.455 | 34.810 | 1.00 | 39.33 | C |
| ATOM | 8569 | O | GLN | E | 38 | −0.868 | 34.969 | 35.751 | 1.00 | 51.07 | O |
| ATOM | 8570 | N | SER | E | 39 | −0.741 | 35.295 | 33.587 | 1.00 | 42.44 | N |
| ATOM | 8572 | CA | SER | E | 39 | −1.905 | 34.453 | 33.408 | 1.00 | 47.12 | C |
| ATOM | 8574 | CB | SER | E | 39 | −3.154 | 35.321 | 33.433 | 1.00 | 52.18 | C |
| ATOM | 8577 | OG | SER | E | 39 | −4.341 | 34.558 | 33.365 | 1.00 | 53.70 | O |
| ATOM | 8579 | C | SER | E | 39 | −1.776 | 33.761 | 32.070 | 1.00 | 53.05 | C |
| ATOM | 8580 | O | SER | E | 39 | −1.014 | 34.206 | 31.191 | 1.00 | 53.76 | O |
| ATOM | 8581 | N | MET | E | 40 | −2.520 | 32.672 | 31.907 | 1.00 | 46.63 | N |
| ATOM | 8583 | CA | MET | E | 40 | −2.692 | 32.133 | 30.571 | 1.00 | 45.12 | C |
| ATOM | 8585 | CB | MET | E | 40 | −2.557 | 30.638 | 30.588 | 1.00 | 41.51 | C |
| ATOM | 8588 | CG | MET | E | 40 | −1.246 | 30.199 | 31.128 | 1.00 | 36.16 | C |
| ATOM | 8591 | SD | MET | E | 40 | −1.089 | 28.401 | 30.880 | 1.00 | 46.04 | S |
| ATOM | 8592 | CE | MET | E | 40 | −2.063 | 27.895 | 32.122 | 1.00 | 37.38 | C |
| ATOM | 8596 | C | MET | E | 40 | −4.073 | 32.482 | 30.113 | 1.00 | 43.80 | C |
| ATOM | 8597 | O | MET | E | 40 | −4.844 | 32.963 | 30.911 | 1.00 | 47.87 | O |
| ATOM | 8598 | N | ASP | E | 41 | −4.365 | 32.247 | 28.839 | 1.00 | 51.73 | N |
| ATOM | 8600 | CA | ASP | E | 41 | −5.662 | 32.590 | 28.251 | 1.00 | 56.52 | C |
| ATOM | 8602 | CB | ASP | E | 41 | −5.514 | 32.914 | 26.762 | 1.00 | 58.63 | C |
| ATOM | 8605 | CG | ASP | E | 41 | −5.140 | 31.695 | 25.928 | 1.00 | 51.10 | C |
| ATOM | 8606 | OD1 | ASP | E | 41 | −6.023 | 30.994 | 25.378 | 1.00 | 45.20 | O |
| ATOM | 8607 | OD2 | ASP | E | 41 | −3.950 | 31.383 | 25.751 | 1.00 | 50.20 | O |
| ATOM | 8608 | C | ASP | E | 41 | −6.637 | 31.429 | 28.414 | 1.00 | 55.65 | C |
| ATOM | 8609 | O | ASP | E | 41 | −6.247 | 30.354 | 28.867 | 1.00 | 51.54 | O |
| ATOM | 8610 | N | SER | E | 42 | −7.895 | 31.656 | 28.046 | 1.00 | 59.62 | N |
| ATOM | 8612 | CA | SER | E | 42 | −8.947 | 30.655 | 28.195 | 1.00 | 54.70 | C |
| ATOM | 8614 | CB | SER | E | 42 | −10.103 | 30.951 | 27.229 | 1.00 | 62.16 | C |
| ATOM | 8617 | OG | SER | E | 42 | −10.842 | 32.116 | 27.608 | 1.00 | 85.38 | O |
| ATOM | 8619 | C | SER | E | 42 | −8.319 | 29.315 | 27.891 | 1.00 | 54.32 | C |
| ATOM | 8620 | O | SER | E | 42 | −8.274 | 28.443 | 28.745 | 1.00 | 52.46 | O |
| ATOM | 8621 | N | SER | E | 43 | −7.792 | 29.147 | 26.681 | 1.00 | 63.60 | N |
| ATOM | 8623 | CA | SER | E | 43 | −6.977 | 27.975 | 26.382 | 1.00 | 59.58 | C |
| ATOM | 8625 | CB | SER | E | 43 | −6.527 | 27.909 | 24.945 | 1.00 | 56.13 | C |
| ATOM | 8628 | OG | SER | E | 43 | −5.602 | 26.833 | 25.015 | 1.00 | 72.85 | O |
| ATOM | 8630 | C | SER | E | 43 | −5.690 | 27.986 | 27.172 | 1.00 | 59.27 | C |
| ATOM | 8631 | O | SER | E | 43 | −5.455 | 28.879 | 27.994 | 1.00 | 69.85 | O |
| ATOM | 8632 | N | HIS | E | 44 | −4.791 | 27.046 | 26.924 | 1.00 | 45.40 | N |
| ATOM | 8634 | CA | HIS | E | 44 | −3.583 | 27.192 | 27.716 | 1.00 | 45.99 | C |
| ATOM | 8636 | CB | HIS | E | 44 | −3.473 | 26.107 | 28.787 | 1.00 | 45.18 | C |
| ATOM | 8639 | CG | HIS | E | 44 | −4.435 | 26.264 | 29.928 | 1.00 | 47.87 | C |
| ATOM | 8640 | ND1 | HIS | E | 44 | −4.505 | 25.359 | 30.963 | 1.00 | 52.33 | N |
| ATOM | 8642 | CE1 | HIS | E | 44 | −5.430 | 25.745 | 31.824 | 1.00 | 59.59 | C |
| ATOM | 8644 | NE2 | HIS | E | 44 | −5.989 | 26.850 | 31.368 | 1.00 | 60.51 | N |
| ATOM | 8646 | CD2 | HIS | E | 44 | −5.402 | 27.182 | 30.170 | 1.00 | 63.40 | C |
| ATOM | 8648 | C | HIS | E | 44 | −2.407 | 27.253 | 26.757 | 1.00 | 48.62 | C |
| ATOM | 8649 | O | HIS | E | 44 | −1.333 | 26.651 | 26.940 | 1.00 | 49.81 | O |
| ATOM | 8650 | N | VAL | E | 45 | −2.652 | 28.033 | 25.719 | 1.00 | 44.25 | N |
| ATOM | 8652 | CA | VAL | E | 45 | −1.772 | 28.054 | 24.571 | 1.00 | 47.02 | C |
| ATOM | 8654 | CB | VAL | E | 45 | −2.559 | 28.218 | 23.281 | 1.00 | 48.24 | C |
| ATOM | 8656 | CG1 | VAL | E | 45 | −1.607 | 28.767 | 22.217 | 1.00 | 58.40 | C |
| ATOM | 8660 | CG2 | VAL | E | 45 | −3.158 | 26.864 | 22.863 | 1.00 | 43.87 | C |
| ATOM | 8664 | C | VAL | E | 45 | −0.818 | 29.236 | 24.676 | 1.00 | 41.79 | C |
| ATOM | 8665 | O | VAL | E | 45 | 0.316 | 29.188 | 24.187 | 1.00 | 42.26 | O |
| ATOM | 8666 | N | SER | E | 46 | −1.311 | 30.298 | 25.295 | 1.00 | 29.46 | N |
| ATOM | 8668 | CA | SER | E | 46 | −0.527 | 31.497 | 25.478 | 1.00 | 31.14 | C |
| ATOM | 8670 | CB | SER | E | 46 | −1.090 | 32.632 | 24.620 | 1.00 | 38.02 | C |
| ATOM | 8673 | OG | SER | E | 46 | −1.701 | 33.653 | 25.372 | 1.00 | 40.00 | O |
| ATOM | 8675 | C | SER | E | 46 | −0.427 | 31.933 | 26.930 | 1.00 | 36.15 | C |
| ATOM | 8676 | O | SER | E | 46 | −1.284 | 31.639 | 27.780 | 1.00 | 29.59 | O |
| ATOM | 8677 | N | LEU | E | 47 | 0.627 | 32.677 | 27.224 | 1.00 | 33.39 | N |
| ATOM | 8679 | CA | LEU | E | 47 | 0.769 | 33.101 | 28.603 | 1.00 | 43.12 | C |
| ATOM | 8681 | CB | LEU | E | 47 | 1.737 | 32.182 | 29.350 | 1.00 | 44.68 | C |
| ATOM | 8684 | CG | LEU | E | 47 | 2.050 | 32.699 | 30.756 | 1.00 | 43.02 | C |
| ATOM | 8686 | CD1 | LEU | E | 47 | 1.646 | 31.712 | 31.834 | 1.00 | 37.82 | C |
| ATOM | 8690 | CD2 | LEU | E | 47 | 3.513 | 32.970 | 30.872 | 1.00 | 58.15 | C |
| ATOM | 8694 | C | LEU | E | 47 | 1.322 | 34.482 | 28.600 | 1.00 | 38.69 | C |
| ATOM | 8695 | O | LEU | E | 47 | 2.242 | 34.785 | 27.850 | 1.00 | 47.48 | O |
| ATOM | 8696 | N | VAL | E | 48 | 0.763 | 35.341 | 29.417 | 1.00 | 35.63 | N |
| ATOM | 8698 | CA | VAL | E | 48 | 1.276 | 36.702 | 29.422 | 1.00 | 35.83 | C |
| ATOM | 8700 | CB | VAL | E | 48 | 0.142 | 37.698 | 29.139 | 1.00 | 30.59 | C |
| ATOM | 8702 | CG1 | VAL | E | 48 | −0.800 | 37.713 | 30.272 | 1.00 | 40.37 | C |
| ATOM | 8706 | CG2 | VAL | E | 48 | 0.687 | 39.108 | 29.022 | 1.00 | 48.63 | C |
| ATOM | 8710 | C | VAL | E | 48 | 1.924 | 37.010 | 30.776 | 1.00 | 33.50 | C |
| ATOM | 8711 | O | VAL | E | 48 | 1.565 | 36.447 | 31.800 | 1.00 | 36.40 | O |
| ATOM | 8712 | N | GLN | E | 49 | 2.861 | 37.940 | 30.788 | 1.00 | 32.92 | N |
| ATOM | 8714 | CA | GLN | E | 49 | 3.670 | 38.185 | 31.967 | 1.00 | 35.63 | C |
| ATOM | 8716 | CB | GLN | E | 49 | 4.881 | 37.245 | 31.968 | 1.00 | 42.55 | C |
| ATOM | 8719 | CG | GLN | E | 49 | 5.498 | 36.939 | 33.371 | 1.00 | 54.05 | C |
| ATOM | 8722 | CD | GLN | E | 49 | 6.757 | 37.737 | 33.538 | 1.00 | 65.22 | C |
| ATOM | 8723 | OE1 | GLN | E | 49 | 7.823 | 37.346 | 33.027 | 1.00 | 57.85 | O |
| ATOM | 8724 | NE2 | GLN | E | 49 | 6.592 | 38.955 | 34.046 | 1.00 | 53.40 | N |
| ATOM | 8727 | C | GLN | E | 49 | 4.132 | 39.639 | 32.023 | 1.00 | 39.03 | C |
| ATOM | 8728 | O | GLN | E | 49 | 4.923 | 40.117 | 31.202 | 1.00 | 42.14 | O |
| ATOM | 8729 | N | LEU | E | 50 | 3.643 | 40.329 | 33.040 | 1.00 | 41.12 | N |
| ATOM | 8731 | CA | LEU | E | 50 | 3.795 | 41.765 | 33.173 | 1.00 | 38.16 | C |
| ATOM | 8733 | CB | LEU | E | 50 | 2.441 | 42.431 | 33.346 | 1.00 | 36.48 | C |
| ATOM | 8736 | CG | LEU | E | 50 | 2.467 | 43.823 | 33.959 | 1.00 | 39.24 | C |
| ATOM | 8738 | CD1 | LEU | E | 50 | 2.754 | 44.829 | 32.876 | 1.00 | 58.35 | C |
| ATOM | 8742 | CD2 | LEU | E | 50 | 1.117 | 44.127 | 34.510 | 1.00 | 36.77 | C |
| ATOM | 8746 | C | LEU | E | 50 | 4.618 | 41.961 | 34.428 | 1.00 | 41.02 | C |
| ATOM | 8747 | O | LEU | E | 50 | 4.388 | 41.339 | 35.458 | 1.00 | 41.05 | O |
| ATOM | 8748 | N | THR | E | 51 | 5.654 | 42.766 | 34.272 | 1.00 | 45.93 | N |
| ATOM | 8750 | CA | THR | E | 51 | 6.543 | 43.165 | 35.346 | 1.00 | 40.44 | C |
| ATOM | 8752 | CB | THR | E | 51 | 7.931 | 42.661 | 35.016 | 1.00 | 43.64 | C |
| ATOM | 8754 | OG1 | THR | E | 51 | 7.998 | 41.241 | 35.180 | 1.00 | 40.36 | O |
| ATOM | 8756 | CG2 | THR | E | 51 | 8.949 | 43.215 | 35.990 | 1.00 | 63.69 | C |
| ATOM | 8760 | C | THR | E | 51 | 6.549 | 44.691 | 35.368 | 1.00 | 39.17 | C |
| ATOM | 8761 | O | THR | E | 51 | 6.762 | 45.314 | 34.342 | 1.00 | 40.95 | O |
| ATOM | 8762 | N | LEU | E | 52 | 6.211 | 45.294 | 36.501 | 1.00 | 37.57 | N |
| ATOM | 8764 | CA | LEU | E | 52 | 6.352 | 46.731 | 36.685 | 1.00 | 40.31 | C |
| ATOM | 8766 | CB | LEU | E | 52 | 4.974 | 47.407 | 36.705 | 1.00 | 40.24 | C |
| ATOM | 8769 | CG | LEU | E | 52 | 3.991 | 47.447 | 35.538 | 1.00 | 39.63 | C |
| ATOM | 8771 | CD1 | LEU | E | 52 | 2.683 | 48.234 | 35.880 | 1.00 | 27.18 | C |
| ATOM | 8775 | CD2 | LEU | E | 52 | 4.646 | 48.033 | 34.366 | 1.00 | 28.47 | C |
| ATOM | 8779 | C | LEU | E | 52 | 7.188 | 47.051 | 37.967 | 1.00 | 41.72 | C |
| ATOM | 8780 | O | LEU | E | 52 | 6.719 | 46.964 | 39.113 | 1.00 | 34.89 | O |
| ATOM | 8781 | N | ARG | E | 53 | 8.455 | 47.404 | 37.761 | 1.00 | 42.50 | N |
| ATOM | 8783 | CA | ARG | E | 53 | 9.393 | 47.613 | 38.859 | 1.00 | 41.84 | C |
| ATOM | 8785 | CB | ARG | E | 53 | 10.820 | 47.767 | 38.309 | 1.00 | 36.70 | C |
| ATOM | 8788 | CG | ARG | E | 53 | 11.394 | 46.544 | 37.584 | 1.00 | 45.48 | C |
| ATOM | 8791 | CD | ARG | E | 53 | 12.787 | 46.724 | 36.878 | 1.00 | 69.62 | C |
| ATOM | 8794 | NE | ARG | E | 53 | 12.823 | 47.496 | 35.623 | 1.00 | 72.26 | N |
| ATOM | 8796 | CZ | ARG | E | 53 | 12.974 | 48.840 | 35.550 | 1.00 | 90.43 | C |
| ATOM | 8797 | NH1 | ARG | E | 53 | 13.089 | 49.624 | 36.640 | 1.00 | 66.05 | N |
| ATOM | 8800 | NH2 | ARG | E | 53 | 12.988 | 49.431 | 34.356 | 1.00 | 86.14 | N |
| ATOM | 8803 | C | ARG | E | 53 | 8.981 | 48.869 | 39.633 | 1.00 | 44.63 | C |
| ATOM | 8804 | O | ARG | E | 53 | 8.494 | 49.834 | 39.054 | 1.00 | 44.48 | O |
| ATOM | 8805 | N | SER | E | 54 | 9.203 | 48.863 | 40.947 | 1.00 | 48.31 | N |
| ATOM | 8807 | CA | SER | E | 54 | 8.502 | 49.764 | 41.854 | 1.00 | 47.67 | C |
| ATOM | 8809 | CB | SER | E | 54 | 8.535 | 49.236 | 43.283 | 1.00 | 49.97 | C |
| ATOM | 8812 | OG | SER | E | 54 | 9.773 | 48.645 | 43.582 | 1.00 | 51.18 | O |
| ATOM | 8814 | C | SER | E | 54 | 9.094 | 51.151 | 41.782 | 1.00 | 43.50 | C |
| ATOM | 8815 | O | SER | E | 54 | 8.390 | 52.138 | 41.765 | 1.00 | 31.95 | O |
| ATOM | 8816 | N | GLU | E | 55 | 10.396 | 51.193 | 41.573 | 1.00 | 51.52 | N |
| ATOM | 8818 | CA | GLU | E | 55 | 11.122 | 52.449 | 41.446 | 1.00 | 53.61 | C |
| ATOM | 8820 | CB | GLU | E | 55 | 12.615 | 52.202 | 41.723 | 1.00 | 49.26 | C |
| ATOM | 8823 | CG | GLU | E | 55 | 12.844 | 50.837 | 42.388 | 1.00 | 71.41 | C |
| ATOM | 8826 | CD | GLU | E | 55 | 12.755 | 49.640 | 41.405 | 1.00 | 97.95 | C |
| ATOM | 8827 | OE1 | GLU | E | 55 | 13.101 | 49.813 | 40.201 | 1.00 | 116.23 | O |
| ATOM | 8828 | OE2 | GLU | E | 55 | 12.374 | 48.497 | 41.797 | 1.00 | 60.58 | O |
| ATOM | 8829 | C | GLU | E | 55 | 10.858 | 53.133 | 40.101 | 1.00 | 49.86 | C |
| ATOM | 8830 | O | GLU | E | 55 | 11.256 | 54.282 | 39.904 | 1.00 | 55.84 | O |
| ATOM | 8831 | N | GLY | E | 56 | 10.092 | 52.499 | 39.218 | 1.00 | 44.45 | N |
| ATOM | 8833 | CA | GLY | E | 56 | 9.608 | 53.204 | 38.036 | 1.00 | 39.13 | C |
| ATOM | 8836 | C | GLY | E | 56 | 8.230 | 53.847 | 38.172 | 1.00 | 37.60 | C |
| ATOM | 8837 | O | GLY | E | 56 | 7.760 | 54.628 | 37.321 | 1.00 | 31.61 | O |
| ATOM | 8838 | N | PHE | E | 57 | 7.559 | 53.588 | 39.287 | 1.00 | 41.45 | N |
| ATOM | 8840 | CA | PHE | E | 57 | 6.467 | 54.488 | 39.637 | 1.00 | 38.21 | C |
| ATOM | 8842 | CB | PHE | E | 57 | 5.516 | 53.875 | 40.620 | 1.00 | 37.09 | C |
| ATOM | 8845 | CG | PHE | E | 57 | 4.780 | 52.712 | 40.063 | 1.00 | 39.16 | C |
| ATOM | 8846 | CD1 | PHE | E | 57 | 5.423 | 51.489 | 39.907 | 1.00 | 44.46 | C |
| ATOM | 8848 | CE1 | PHE | E | 57 | 4.738 | 50.364 | 39.437 | 1.00 | 51.51 | C |
| ATOM | 8850 | CZ | PHE | E | 57 | 3.414 | 50.457 | 39.092 | 1.00 | 28.48 | C |
| ATOM | 8852 | CE2 | PHE | E | 57 | 2.771 | 51.720 | 39.193 | 1.00 | 50.21 | C |
| ATOM | 8854 | CD2 | PHE | E | 57 | 3.454 | 52.831 | 39.681 | 1.00 | 46.13 | C |
| ATOM | 8856 | C | PHE | E | 57 | 6.997 | 55.780 | 40.156 | 1.00 | 30.69 | C |
| ATOM | 8857 | O | PHE | E | 57 | 8.183 | 56.028 | 40.089 | 1.00 | 47.11 | O |
| ATOM | 8858 | N | ASP | E | 58 | 6.090 | 56.683 | 40.455 | 1.00 | 35.28 | N |
| ATOM | 8860 | CA | ASP | E | 58 | 6.462 | 58.055 | 40.753 | 1.00 | 39.41 | C |
| ATOM | 8862 | CB | ASP | E | 58 | 5.607 | 59.019 | 39.947 | 1.00 | 37.27 | C |
| ATOM | 8865 | CG | ASP | E | 58 | 5.922 | 58.995 | 38.471 | 1.00 | 40.02 | C |
| ATOM | 8866 | OD1 | ASP | E | 58 | 6.830 | 59.733 | 38.046 | 1.00 | 56.72 | O |
| ATOM | 8867 | OD2 | ASP | E | 58 | 5.247 | 58.364 | 37.633 | 1.00 | 41.53 | O |
| ATOM | 8868 | C | ASP | E | 58 | 6.133 | 58.201 | 42.228 | 1.00 | 44.50 | C |
| ATOM | 8869 | O | ASP | E | 58 | 6.606 | 59.124 | 42.889 | 1.00 | 45.90 | O |
| ATOM | 8870 | N | THR | E | 59 | 5.326 | 57.257 | 42.721 | 1.00 | 42.27 | N |
| ATOM | 8872 | CA | THR | E | 59 | 5.087 | 57.097 | 44.143 | 1.00 | 40.97 | C |
| ATOM | 8874 | CB | THR | E | 59 | 3.884 | 57.937 | 44.537 | 1.00 | 36.33 | C |
| ATOM | 8876 | OG1 | THR | E | 59 | 4.242 | 59.320 | 44.616 | 1.00 | 54.50 | O |
| ATOM | 8878 | CG2 | THR | E | 59 | 3.400 | 57.591 | 45.924 | 1.00 | 52.02 | C |
| ATOM | 8882 | C | THR | E | 89 | 4.791 | 55.620 | 44.360 | 1.00 | 43.65 | C |
| ATOM | 8883 | O | THR | E | 59 | 3.958 | 55.078 | 43.652 | 1.00 | 57.08 | O |
| ATOM | 8884 | N | TYR | E | 60 | 5.476 | 54.969 | 45.299 | 1.00 | 40.11 | N |
| ATOM | 8886 | CA | TYR | E | 60 | 5.317 | 53.546 | 45.522 | 1.00 | 37.66 | C |
| ATOM | 8888 | CB | TYR | E | 60 | 6.318 | 52.717 | 44.726 | 1.00 | 37.63 | C |
| ATOM | 8891 | CG | TYR | E | 60 | 5.894 | 51.255 | 44.650 | 1.00 | 40.90 | C |
| ATOM | 8892 | CD1 | TYR | E | 60 | 5.055 | 50.817 | 43.648 | 1.00 | 34.43 | C |
| ATOM | 8894 | CE1 | TYR | E | 60 | 4.589 | 49.534 | 43.599 | 1.00 | 41.55 | C |
| ATOM | 8896 | CZ | TYR | E | 60 | 5.003 | 48.618 | 44.530 | 1.00 | 54.83 | C |
| ATOM | 8897 | OH | TYR | E | 60 | 4.510 | 47.327 | 44.472 | 1.00 | 38.45 | O |
| ATOM | 8899 | CE2 | TYR | E | 60 | 5.873 | 49.013 | 45.529 | 1.00 | 34.58 | C |
| ATOM | 8901 | CD2 | TYR | E | 60 | 6.293 | 50.329 | 45.600 | 1.00 | 38.87 | C |
| ATOM | 8903 | C | TYR | E | 60 | 5.510 | 53.199 | 46.984 | 1.00 | 44.10 | C |
| ATOM | 8904 | O | TYR | E | 60 | 6.543 | 53.482 | 47.579 | 1.00 | 54.26 | O |
| ATOM | 8905 | N | ARG | E | 61 | 4.513 | 52.529 | 47.534 | 1.00 | 40.08 | N |
| ATOM | 8907 | CA | ARG | E | 61 | 4.534 | 52.102 | 48.908 | 1.00 | 38.54 | C |
| ATOM | 8909 | CB | ARG | E | 61 | 3.783 | 53.105 | 49.765 | 1.00 | 43.10 | C |
| ATOM | 8912 | CG | ARG | E | 61 | 3.645 | 52.630 | 51.203 | 1.00 | 49.71 | C |
| ATOM | 8915 | CD | ARG | E | 61 | 2.792 | 53.520 | 52.093 | 1.00 | 63.60 | C |
| ATOM | 8918 | NE | ARG | E | 61 | 2.445 | 52.854 | 53.350 | 1.00 | 59.47 | N |
| ATOM | 8920 | CZ | ARG | E | 61 | 1.808 | 53.451 | 54.338 | 1.00 | 49.65 | C |
| ATOM | 8921 | NH1 | ARG | E | 61 | 1.440 | 54.717 | 54.227 | 1.00 | 61.85 | N |
| ATOM | 8924 | NH2 | ARG | E | 61 | 1.468 | 52.768 | 55.409 | 1.00 | 58.49 | N |
| ATOM | 8927 | C | ARG | E | 61 | 3.822 | 50.765 | 49.015 | 1.00 | 43.55 | C |
| ATOM | 8928 | O | ARG | E | 61 | 2.633 | 50.650 | 48.674 | 1.00 | 48.46 | O |
| ATOM | 8929 | N | CYS | E | 62 | 4.512 | 49.750 | 49.528 | 1.00 | 38.72 | N |
| ATOM | 8931 | CA | CYS | E | 62 | 3.843 | 48.475 | 49.678 | 1.00 | 32.14 | C |
| ATOM | 8933 | CB | CYS | E | 62 | 4.356 | 47.516 | 48.650 | 1.00 | 37.19 | C |
| ATOM | 8936 | SG | CYS | E | 62 | 3.436 | 45.973 | 48.640 | 1.00 | 46.58 | S |
| ATOM | 8937 | C | CYS | E | 62 | 4.192 | 47.934 | 51.011 | 1.00 | 32.91 | C |
| ATOM | 8938 | O | CYS | E | 62 | 5.363 | 47.689 | 51.237 | 1.00 | 45.76 | O |
| ATOM | 8939 | N | ASP | E | 63 | 3.215 | 47.798 | 51.900 | 1.00 | 43.40 | N |
| ATOM | 8941 | CA | ASP | E | 63 | 3.467 | 47.319 | 53.273 | 1.00 | 46.69 | C |
| ATOM | 8943 | CB | ASP | E | 63 | 2.528 | 47.971 | 54.271 | 1.00 | 38.94 | C |
| ATOM | 8946 | CG | ASP | E | 63 | 2.803 | 49.444 | 54.416 | 1.00 | 59.18 | C |
| ATOM | 8947 | OD1 | ASP | E | 63 | 4.011 | 49.831 | 54.298 | 1.00 | 50.53 | O |
| ATOM | 8948 | OD2 | ASP | E | 63 | 1.857 | 50.248 | 54.664 | 1.00 | 38.48 | O |
| ATOM | 8949 | C | ASP | E | 63 | 3.346 | 45.820 | 53.447 | 1.00 | 42.08 | C |
| ATOM | 8950 | O | ASP | E | 63 | 4.167 | 45.247 | 54.105 | 1.00 | 55.94 | O |
| ATOM | 8951 | N | ARG | E | 64 | 2.347 | 45.188 | 52.848 | 1.00 | 52.06 | N |
| ATOM | 8953 | CA | ARG | E | 64 | 2.270 | 43.732 | 52.765 | 1.00 | 54.86 | C |
| ATOM | 8955 | CB | ARG | E | 64 | 1.215 | 43.240 | 53.758 | 1.00 | 60.72 | C |
| ATOM | 8958 | CG | ARG | E | 64 | 1.108 | 41.704 | 53.772 | 1.00 | 91.55 | C |
| ATOM | 8961 | CD | ARG | E | 64 | −0.248 | 41.107 | 54.208 | 1.00 | 110.94 | C |
| ATOM | 8964 | NE | ARG | E | 64 | −1.020 | 41.984 | 55.092 | 1.00 | 124.30 | N |
| ATOM | 8966 | CZ | ARG | E | 64 | −0.671 | 42.341 | 56.326 | 1.00 | 120.54 | C |
| ATOM | 8967 | NH1 | ARG | E | 64 | 0.459 | 41.921 | 56.891 | 1.00 | 114.84 | N |
| ATOM | 8970 | NH2 | ARG | E | 64 | −1.478 | 43.143 | 57.003 | 1.00 | 119.82 | N |
| ATOM | 8973 | C | ARG | E | 64 | 1.956 | 43.238 | 51.332 | 1.00 | 44.93 | C |
| ATOM | 8974 | O | ARG | E | 64 | 1.081 | 43.754 | 50.670 | 1.00 | 43.76 | O |
| ATOM | 8975 | N | ASN | E | 65 | 2.733 | 42.297 | 50.810 | 1.00 | 43.96 | N |
| ATOM | 8977 | CA | ASN | E | 65 | 2.338 | 41.510 | 49.647 | 1.00 | 36.78 | C |
| ATOM | 8979 | CB | ASN | E | 65 | 2.997 | 40.130 | 49.683 | 1.00 | 30.80 | C |
| ATOM | 8982 | CG | ASN | E | 65 | 4.513 | 40.185 | 49.579 | 1.00 | 45.91 | C |
| ATOM | 8983 | OD1 | ASN | E | 65 | 5.160 | 39.217 | 49.955 | 1.00 | 58.19 | O |
| ATOM | 8984 | ND2 | ASN | E | 65 | 5.087 | 41.302 | 49.100 | 1.00 | 52.05 | N |
| ATOM | 8987 | C | ASN | E | 65 | 0.838 | 41.282 | 49.562 | 1.00 | 39.84 | C |
| ATOM | 8988 | O | ASN | E | 65 | 0.196 | 41.025 | 50.564 | 1.00 | 35.28 | O |
| ATOM | 8989 | N | LEU | E | 66 | 0.309 | 41.336 | 48.341 | 1.00 | 51.37 | N |
| ATOM | 8991 | CA | LEU | E | 66 | −1.109 | 41.603 | 48.042 | 1.00 | 49.32 | C |
| ATOM | 8993 | CB | LEU | E | 66 | −1.375 | 43.101 | 47.847 | 1.00 | 42.80 | C |
| ATOM | 8996 | CG | LEU | E | 66 | −2.549 | 43.653 | 48.634 | 1.00 | 49.86 | C |
| ATOM | 8998 | CD1 | LEU | E | 66 | −2.140 | 43.781 | 50.094 | 1.00 | 70.44 | C |
| ATOM | 9002 | CD2 | LEU | E | 66 | −2.984 | 44.994 | 48.059 | 1.00 | 67.90 | C |
| ATOM | 9006 | C | LEU | E | 66 | −1.353 | 40.919 | 46.718 | 1.00 | 46.82 | C |
| ATOM | 9007 | O | LEU | E | 66 | −0.539 | 41.042 | 45.800 | 1.00 | 51.38 | O |
| ATOM | 9008 | N | ALA | E | 67 | −2.436 | 40.168 | 46.639 | 1.00 | 43.59 | N |
| ATOM | 9010 | CA | ALA | E | 67 | −2.738 | 39.431 | 45.419 | 1.00 | 47.24 | C |
| ATOM | 9012 | CB | ALA | E | 67 | −2.655 | 37.923 | 45.628 | 1.00 | 31.90 | C |
| ATOM | 9016 | C | ALA | E | 67 | −4.164 | 39.831 | 45.046 | 1.00 | 53.16 | C |
| ATOM | 9017 | O | ALA | E | 67 | −5.132 | 39.337 | 45.609 | 1.00 | 56.13 | O |
| ATOM | 9018 | N | MET | E | 68 | −4.292 | 40.765 | 44.119 | 1.00 | 52.75 | N |
| ATOM | 9020 | CA | MET | E | 68 | −5.598 | 41.285 | 43.819 | 1.00 | 49.51 | C |
| ATOM | 9022 | CB | MET | E | 68 | −5.500 | 42.787 | 43.614 | 1.00 | 53.23 | C |
| ATOM | 9025 | CG | MET | E | 68 | −5.346 | 43.575 | 44.902 | 1.00 | 53.20 | C |
| ATOM | 9028 | SD | MET | E | 68 | −5.270 | 45.334 | 44.523 | 1.00 | 63.60 | S |
| ATOM | 9029 | CE | MET | E | 68 | −3.897 | 45.324 | 43.342 | 1.00 | 56.33 | C |
| ATOM | 9033 | C | MET | E | 68 | −6.107 | 40.575 | 42.579 | 1.00 | 48.33 | C |
| ATOM | 9034 | O | MET | E | 68 | −5.482 | 40.592 | 41.523 | 1.00 | 52.15 | O |
| ATOM | 9035 | N | GLY | E | 69 | −7.241 | 39.916 | 42.764 | 1.00 | 44.97 | N |
| ATOM | 9037 | CA | GLY | E | 69 | −8.054 | 39.393 | 41.693 | 1.00 | 42.15 | C |
| ATOM | 9040 | C | GLY | E | 69 | −8.931 | 40.430 | 41.006 | 1.00 | 39.37 | C |
| ATOM | 9041 | O | GLY | E | 69 | −9.572 | 41.229 | 41.647 | 1.00 | 36.95 | O |
| ATOM | 9042 | N | VAL | E | 70 | −8.983 | 40.395 | 39.681 | 1.00 | 40.93 | N |
| ATOM | 9044 | CA | VAL | E | 70 | −9.428 | 41.535 | 38.937 | 1.00 | 40.58 | C |
| ATOM | 9046 | CB | VAL | E | 70 | −8.305 | 42.516 | 38.663 | 1.00 | 44.32 | C |
| ATOM | 9048 | CG1 | VAL | E | 70 | −8.790 | 43.588 | 37.718 | 1.00 | 57.00 | C |
| ATOM | 9052 | CG2 | VAL | E | 70 | −7.826 | 43.168 | 39.954 | 1.00 | 50.56 | C |
| ATOM | 9056 | C | VAL | E | 70 | −9.974 | 41.020 | 37.651 | 1.00 | 42.26 | C |
| ATOM | 9057 | O | VAL | E | 70 | −9.320 | 40.229 | 36.973 | 1.00 | 44.93 | O |
| ATOM | 9058 | N | ASN | E | 71 | −11.180 | 41.487 | 37.347 | 1.00 | 47.76 | N |
| ATOM | 9060 | CA | ASN | E | 71 | −11.807 | 41.287 | 36.052 | 1.00 | 46.79 | C |
| ATOM | 9062 | CB | ASN | E | 71 | −13.306 | 41.538 | 36.143 | 1.00 | 54.04 | C |
| ATOM | 9065 | CG | ASN | E | 71 | −14.027 | 41.137 | 34.891 | 1.00 | 55.20 | C |
| ATOM | 9066 | OD1 | ASN | E | 71 | −13.843 | 41.756 | 33.846 | 1.00 | 73.20 | O |
| ATOM | 9067 | ND2 | ASN | E | 71 | −14.787 | 40.047 | 34.967 | 1.00 | 61.17 | N |
| ATOM | 9070 | C | ASN | E | 71 | −11.298 | 42.364 | 35.153 | 1.00 | 47.70 | C |
| ATOM | 9071 | O | ASN | E | 71 | −11.559 | 43.539 | 35.393 | 1.00 | 44.80 | O |
| ATOM | 9072 | N | LEU | E | 72 | −10.542 | 41.973 | 34.144 | 1.00 | 46.29 | N |
| ATOM | 9074 | CA | LEU | E | 72 | −9.673 | 42.943 | 33.513 | 1.00 | 44.32 | C |
| ATOM | 9076 | CB | LEU | E | 72 | −8.628 | 42.242 | 32.663 | 1.00 | 44.09 | C |
| ATOM | 9079 | CG | LEU | E | 72 | −7.529 | 41.609 | 33.499 | 1.00 | 50.10 | C |
| ATOM | 9081 | CD1 | LEU | E | 72 | −6.488 | 41.025 | 32.556 | 1.00 | 48.15 | C |
| ATOM | 9085 | CD2 | LEU | E | 72 | −6.950 | 42.661 | 34.435 | 1.00 | 60.80 | C |
| ATOM | 9089 | C | LEU | E | 72 | −10.538 | 43.802 | 32.616 | 1.00 | 43.91 | C |
| ATOM | 9090 | O | LEU | E | 72 | −10.112 | 44.855 | 32.170 | 1.00 | 35.40 | O |
| ATOM | 9091 | N | THR | E | 73 | −11.741 | 43.331 | 32.315 | 1.00 | 47.86 | N |
| ATOM | 9093 | CA | THR | E | 73 | −12.567 | 44.042 | 31.363 | 1.00 | 49.04 | C |
| ATOM | 9095 | CB | THR | E | 73 | −13.695 | 43.140 | 30.787 | 1.00 | 52.96 | C |
| ATOM | 9097 | OG1 | THR | E | 73 | −13.168 | 42.216 | 29.821 | 1.00 | 54.89 | O |
| ATOM | 9099 | CG2 | THR | E | 73 | −14.673 | 43.958 | 29.971 | 1.00 | 59.16 | C |
| ATOM | 9103 | C | THR | E | 73 | −13.109 | 45.171 | 32.217 | 1.00 | 48.48 | C |
| ATOM | 9104 | O | THR | E | 73 | −13.171 | 46.324 | 31.782 | 1.00 | 44.53 | O |
| ATOM | 9105 | N | SER | E | 74 | −13.431 | 44.838 | 33.465 | 1.00 | 47.81 | N |
| ATOM | 9107 | CA | SER | E | 74 | −13.984 | 45.823 | 34.406 | 1.00 | 44.55 | C |
| ATOM | 9109 | CB | SER | E | 74 | −14.395 | 45.184 | 35.761 | 1.00 | 47.62 | C |
| ATOM | 9112 | OG | SER | E | 74 | −15.254 | 44.031 | 35.625 | 1.00 | 48.86 | O |
| ATOM | 9114 | C | SER | E | 74 | −12.936 | 46.904 | 34.606 | 1.00 | 36.01 | C |
| ATOM | 9115 | O | SER | E | 74 | −13.193 | 48.087 | 34.426 | 1.00 | 42.61 | O |
| ATOM | 9116 | N | MET | E | 75 | −11.719 | 46.490 | 34.914 | 1.00 | 36.80 | N |
| ATOM | 9118 | CA | MET | E | 75 | −10.614 | 47.434 | 35.023 | 1.00 | 36.46 | C |
| ATOM | 9120 | CB | MET | E | 75 | −9.313 | 46.725 | 35.373 | 1.00 | 29.37 | C |
| ATOM | 9123 | CG | MET | E | 75 | −8.267 | 47.682 | 35.936 | 1.00 | 31.42 | C |
| ATOM | 9126 | SD | MET | E | 75 | −6.910 | 46.684 | 36.574 | 1.00 | 42.85 | S |
| ATOM | 9127 | CE | MET | E | 75 | −5.786 | 47.960 | 37.078 | 1.00 | 25.47 | C |
| ATOM | 9131 | C | MET | E | 75 | −10.390 | 48.320 | 33.797 | 1.00 | 40.87 | C |
| ATOM | 9132 | O | MET | E | 75 | −10.031 | 49.513 | 33.937 | 1.00 | 37.32 | O |
| ATOM | 9133 | N | SER | E | 76 | −10.565 | 47.732 | 32.612 | 1.00 | 38.79 | N |
| ATOM | 9135 | CA | SER | E | 76 | −10.466 | 48.466 | 31.341 | 1.00 | 42.71 | C |
| ATOM | 9137 | CB | SER | E | 76 | −10.690 | 47.516 | 30.159 | 1.00 | 36.73 | C |
| ATOM | 9140 | OG | SER | E | 76 | −10.654 | 48.187 | 28.901 | 1.00 | 58.23 | O |
| ATOM | 9142 | C | SER | E | 76 | −11.467 | 49.636 | 31.214 | 1.00 | 48.15 | C |
| ATOM | 9143 | O | SER | E | 76 | −11.097 | 50.786 | 30.873 | 1.00 | 45.80 | O |
| ATOM | 9144 | N | LYS | E | 77 | −12.739 | 49.298 | 31.423 | 1.00 | 47.29 | N |
| ATOM | 9146 | CA | LYS | E | 77 | −13.821 | 50.262 | 31.351 | 1.00 | 45.89 | C |
| ATOM | 9148 | CB | LYS | E | 77 | −15.157 | 49.632 | 31.744 | 1.00 | 47.18 | C |
| ATOM | 9151 | CG | LYS | E | 77 | −15.633 | 48.477 | 30.873 | 1.00 | 53.25 | C |
| ATOM | 9154 | CD | LYS | E | 77 | −16.861 | 47.806 | 31.522 | 1.00 | 88.73 | C |
| ATOM | 9157 | CE | LYS | E | 77 | −17.585 | 46.870 | 30.552 | 1.00 | 98.75 | C |
| ATOM | 9160 | NZ | LYS | E | 77 | −16.922 | 46.908 | 29.210 | 1.00 | 94.91 | N |
| ATOM | 9164 | C | LYS | E | 77 | −13.439 | 51.327 | 32.359 | 1.00 | 49.05 | C |
| ATOM | 9165 | O | LYS | E | 77 | −13.553 | 52.512 | 32.095 | 1.00 | 51.86 | O |
| ATOM | 9166 | N | ILE | E | 78 | −12.964 | 50.948 | 33.533 | 1.00 | 49.18 | N |
| ATOM | 9168 | CA | ILE | E | 78 | −12.682 | 52.025 | 34.460 | 1.00 | 52.00 | C |
| ATOM | 9170 | CB | ILE | E | 78 | −12.483 | 51.536 | 35.916 | 1.00 | 50.69 | C |
| ATOM | 9172 | CG1 | ILE | E | 78 | −13.859 | 51.344 | 36.536 | 1.00 | 50.00 | C |
| ATOM | 9175 | CD1 | ILE | E | 78 | −13.962 | 50.196 | 37.467 | 1.00 | 69.54 | C |
| ATOM | 9179 | CG2 | ILE | E | 78 | −11.710 | 52.605 | 36.719 | 1.00 | 45.30 | C |
| ATOM | 9183 | C | ILE | E | 78 | −11.559 | 52.926 | 33.938 | 1.00 | 44.03 | C |
| ATOM | 9184 | O | ILE | E | 78 | −11.711 | 54.138 | 33.882 | 1.00 | 40.01 | O |
| ATOM | 9185 | N | LEU | E | 79 | −10.446 | 52.334 | 33.527 | 1.00 | 47.42 | N |
| ATOM | 9187 | CA | LEU | E | 79 | −9.318 | 53.124 | 33.037 | 1.00 | 50.06 | C |
| ATOM | 9189 | CB | LEU | E | 79 | −8.155 | 52.214 | 32.719 | 1.00 | 46.26 | C |
| ATOM | 9192 | CG | LEU | E | 79 | −7.406 | 51.749 | 33.950 | 1.00 | 44.47 | C |
| ATOM | 9194 | CD1 | LEU | E | 79 | −6.294 | 50.836 | 33.523 | 1.00 | 57.56 | C |
| ATOM | 9198 | CD2 | LEU | E | 79 | −6.859 | 52.957 | 34.649 | 1.00 | 59.41 | C |
| ATOM | 9202 | C | LEU | E | 79 | −9.649 | 53.911 | 31.778 | 1.00 | 51.39 | C |
| ATOM | 9203 | O | LEU | E | 79 | −8.971 | 54.878 | 31.421 | 1.00 | 53.75 | O |
| ATOM | 9204 | N | LYS | E | 80 | −10.703 | 53.480 | 31.101 | 1.00 | 52.88 | N |
| ATOM | 9206 | CA | LYS | E | 80 | −11.204 | 54.225 | 29.953 | 1.00 | 51.16 | C |
| ATOM | 9208 | CB | LYS | E | 80 | −12.255 | 53.402 | 29.175 | 1.00 | 43.69 | C |
| ATOM | 9211 | CG | LYS | E | 80 | −11.949 | 53.201 | 27.720 | 1.00 | 49.41 | C |
| ATOM | 9214 | CD | LYS | E | 80 | −11.650 | 51.766 | 27.383 | 1.00 | 69.28 | C |
| ATOM | 9217 | CE | LYS | E | 80 | −11.215 | 51.660 | 25.921 | 1.00 | 80.62 | C |
| ATOM | 9220 | NZ | LYS | E | 80 | −11.364 | 50.259 | 25.414 | 1.00 | 88.13 | N |
| ATOM | 9224 | C | LYS | E | 80 | −11.735 | 55.576 | 30.419 | 1.00 | 48.05 | C |
| ATOM | 9225 | O | LYS | E | 80 | −11.858 | 56.477 | 29.627 | 1.00 | 61.37 | O |
| ATOM | 9226 | N | CYS | E | 81 | −11.940 | 55.774 | 31.715 | 1.00 | 51.44 | N |
| ATOM | 9228 | CA | CYS | E | 81 | −12.393 | 57.070 | 32.207 | 1.00 | 45.90 | C |
| ATOM | 9230 | CB | CYS | E | 81 | −13.284 | 56.879 | 33.412 | 1.00 | 43.63 | C |
| ATOM | 9233 | SG | CYS | E | 81 | −14.576 | 55.732 | 32.968 | 1.00 | 49.43 | S |
| ATOM | 9234 | C | CYS | E | 81 | −11.271 | 57.990 | 32.593 | 1.00 | 45.78 | C |
| ATOM | 9235 | O | CYS | E | 81 | −11.450 | 58.883 | 33.415 | 1.00 | 44.30 | O |
| ATOM | 9236 | N | ALA | E | 82 | −10.107 | 57.745 | 32.015 | 1.00 | 49.72 | N |
| ATOM | 9238 | CA | ALA | E | 82 | −8.891 | 58.407 | 32.457 | 1.00 | 50.58 | C |
| ATOM | 9240 | CB | ALA | E | 82 | −8.008 | 57.420 | 33.155 | 1.00 | 50.79 | C |
| ATOM | 9244 | C | ALA | E | 82 | −8.177 | 58.972 | 31.249 | 1.00 | 46.31 | C |
| ATOM | 9245 | O | ALA | E | 82 | −8.009 | 58.284 | 30.255 | 1.00 | 48.62 | O |
| ATOM | 9246 | N | GLY | E | 83 | −7.726 | 60.212 | 31.348 | 1.00 | 48.98 | N |
| ATOM | 9248 | CA | GLY | E | 83 | −7.038 | 60.881 | 30.253 | 1.00 | 51.71 | C |
| ATOM | 9251 | C | GLY | E | 83 | −5.686 | 60.257 | 29.951 | 1.00 | 49.12 | C |
| ATOM | 9252 | O | GLY | E | 83 | −5.151 | 59.558 | 30.797 | 1.00 | 53.30 | O |
| ATOM | 9253 | N | ASN | E | 84 | −5.134 | 60.460 | 28.761 | 1.00 | 48.19 | N |
| ATOM | 9255 | CA | ASN | E | 84 | −3.815 | 59.914 | 28.487 | 1.00 | 55.20 | C |
| ATOM | 9257 | CB | ASN | E | 84 | −3.530 | 59.837 | 26.992 | 1.00 | 58.60 | C |
| ATOM | 9260 | CG | ASN | E | 84 | −4.217 | 58.647 | 26.348 | 1.00 | 59.76 | C |
| ATOM | 9261 | OD1 | ASN | E | 84 | −4.143 | 57.534 | 26.851 | 1.00 | 64.01 | O |
| ATOM | 9262 | ND2 | ASN | E | 84 | −4.945 | 58.890 | 25.275 | 1.00 | 58.03 | N |
| ATOM | 9265 | C | ASN | E | 84 | −2.733 | 60.674 | 29.229 | 1.00 | 59.41 | C |
| ATOM | 9266 | O | ASN | E | 84 | −1.652 | 60.108 | 29.476 | 1.00 | 57.02 | O |
| ATOM | 9267 | N | GLU | E | 85 | −3.073 | 61.894 | 29.673 | 1.00 | 58.97 | N |
| ATOM | 9269 | CA | GLU | E | 85 | −2.130 | 62.706 | 30.449 | 1.00 | 59.18 | C |
| ATOM | 9271 | CB | GLU | E | 85 | −1.891 | 64.050 | 29.753 | 1.00 | 61.84 | C |
| ATOM | 9274 | CG | GLU | E | 85 | −0.607 | 64.127 | 28.918 | 1.00 | 75.93 | C |
| ATOM | 9277 | CD | GLU | E | 85 | −0.814 | 63.709 | 27.467 | 1.00 | 99.48 | C |
| ATOM | 9278 | OE1 | GLU | E | 85 | −1.715 | 64.289 | 26.814 | 1.00 | 113.52 | O |
| ATOM | 9279 | OE2 | GLU | E | 85 | −0.090 | 62.805 | 26.976 | 1.00 | 102.06 | O |
| ATOM | 9280 | C | GLU | E | 85 | −2.437 | 62.859 | 31.952 | 1.00 | 50.82 | C |
| ATOM | 9281 | O | GLU | E | 85 | −1.634 | 63.400 | 32.710 | 1.00 | 52.29 | O |
| ATOM | 9282 | N | ASP | E | 86 | −3.542 | 62.267 | 32.391 | 1.00 | 43.23 | N |
| ATOM | 9284 | CA | ASP | E | 86 | −3.869 | 62.137 | 33.805 | 1.00 | 40.14 | C |
| ATOM | 9286 | CB | ASP | E | 86 | −5.175 | 61.348 | 33.998 | 1.00 | 38.93 | C |
| ATOM | 9289 | CG | ASP | E | 86 | −6.399 | 62.117 | 33.571 | 1.00 | 34.35 | C |
| ATOM | 9290 | OD1 | ASP | E | 86 | −6.294 | 63.276 | 33.111 | 1.00 | 54.40 | O |
| ATOM | 9291 | OD2 | ASP | E | 86 | −7.538 | 61.648 | 33.705 | 1.00 | 43.74 | O |
| ATOM | 9292 | C | ASP | E | 86 | −2.776 | 61.379 | 34.553 | 1.00 | 45.45 | C |
| ATOM | 9293 | O | ASP | E | 86 | −2.153 | 60.446 | 34.035 | 1.00 | 37.19 | O |
| ATOM | 9294 | N | ILE | E | 87 | −2.587 | 61.802 | 35.796 | 1.00 | 48.46 | N |
| ATOM | 9296 | CA | ILE | E | 87 | −1.872 | 61.057 | 36.812 | 1.00 | 46.75 | C |
| ATOM | 9298 | CB | ILE | E | 87 | −1.352 | 62.028 | 37.907 | 1.00 | 52.56 | C |
| ATOM | 9300 | CG1 | ILE | E | 87 | −0.248 | 62.973 | 37.402 | 1.00 | 54.28 | C |
| ATOM | 9303 | CD1 | ILE | E | 87 | −0.788 | 64.343 | 36.971 | 1.00 | 68.81 | C |
| ATOM | 9307 | CG2 | ILE | E | 87 | −0.860 | 61.252 | 39.123 | 1.00 | 73.14 | C |
| ATOM | 9311 | C | ILE | E | 87 | −2.824 | 60.028 | 37.419 | 1.00 | 46.86 | C |
| ATOM | 9312 | O | ILE | E | 87 | −3.842 | 60.362 | 38.015 | 1.00 | 54.11 | O |
| ATOM | 9313 | N | ILE | E | 88 | −2.465 | 58.759 | 37.288 | 1.00 | 45.17 | N |
| ATOM | 9315 | CA | ILE | E | 88 | −3.223 | 57.680 | 37.874 | 1.00 | 40.37 | C |
| ATOM | 9317 | CB | ILE | E | 88 | −3.291 | 56.561 | 36.884 | 1.00 | 42.46 | C |
| ATOM | 9319 | CG1 | ILE | E | 88 | −3.829 | 57.103 | 35.562 | 1.00 | 46.37 | C |
| ATOM | 9322 | CD1 | ILE | E | 88 | −4.260 | 55.998 | 34.679 | 1.00 | 47.85 | C |
| ATOM | 9326 | CG2 | ILE | E | 88 | −4.142 | 55.407 | 37.432 | 1.00 | 43.16 | C |
| ATOM | 9330 | C | ILE | E | 88 | −2.573 | 57.162 | 39.142 | 1.00 | 49.37 | C |
| ATOM | 9331 | O | ILE | E | 88 | −1.413 | 56.757 | 39.120 | 1.00 | 46.88 | O |
| ATOM | 9332 | N | THR | E | 89 | −3.355 | 57.107 | 40.218 | 1.00 | 50.42 | N |
| ATOM | 9334 | CA | THR | E | 89 | −2.896 | 56.545 | 41.474 | 1.00 | 53.41 | C |
| ATOM | 9336 | CB | THR | E | 89 | −3.050 | 57.584 | 42.548 | 1.00 | 59.99 | C |
| ATOM | 9338 | OG1 | THR | E | 89 | −2.539 | 58.841 | 42.075 | 1.00 | 59.36 | O |
| ATOM | 9340 | CG2 | THR | E | 89 | −2.242 | 57.186 | 43.743 | 1.00 | 68.16 | C |
| ATOM | 9344 | C | THR | E | 89 | −3.708 | 55.336 | 41.883 | 1.00 | 48.39 | C |
| ATOM | 9345 | O | THR | E | 89 | −4.909 | 55.417 | 41.967 | 1.00 | 47.29 | O |
| ATOM | 9346 | N | LEU | E | 90 | −3.062 | 54.199 | 42.089 | 1.00 | 50.23 | N |
| ATOM | 9348 | CA | LEU | E | 90 | −3.762 | 53.024 | 42.576 | 1.00 | 50.40 | C |
| ATOM | 9350 | CB | LEU | E | 90 | −3.190 | 51.763 | 41.951 | 1.00 | 51.21 | C |
| ATOM | 9353 | CG | LEU | E | 90 | −3.198 | 51.874 | 40.442 | 1.00 | 53.41 | C |
| ATOM | 9355 | CD1 | LEU | E | 90 | −2.359 | 50.746 | 39.940 | 1.00 | 28.66 | C |
| ATOM | 9359 | CD2 | LEU | E | 90 | −4.627 | 51.823 | 39.890 | 1.00 | 63.83 | C |
| ATOM | 9363 | C | LEU | E | 90 | −3.528 | 52.912 | 44.061 | 1.00 | 45.14 | C |
| ATOM | 9364 | O | LEU | E | 90 | −2.439 | 53.239 | 44.527 | 1.00 | 42.58 | O |
| ATOM | 9365 | N | ARG | E | 91 | −4.524 | 52.428 | 44.793 | 1.00 | 38.32 | N |
| ATOM | 9367 | CA | ARG | E | 91 | −4.406 | 52.403 | 46.239 | 1.00 | 41.20 | C |
| ATOM | 9369 | CB | ARG | E | 91 | −4.600 | 53.791 | 46.860 | 1.00 | 30.17 | C |
| ATOM | 9372 | CG | ARG | E | 91 | −4.697 | 53.737 | 48.350 | 1.00 | 51.65 | C |
| ATOM | 9375 | CD | ARG | E | 91 | −4.887 | 55.071 | 49.068 | 1.00 | 65.64 | C |
| ATOM | 9378 | NE | ARG | E | 91 | −5.373 | 56.129 | 48.196 | 1.00 | 77.66 | N |
| ATOM | 9380 | CZ | ARG | E | 91 | −4.625 | 57.110 | 47.710 | 1.00 | 82.66 | C |
| ATOM | 9381 | NH1 | ARG | E | 91 | −3.328 | 57.156 | 47.977 | 1.00 | 82.57 | N |
| ATOM | 9384 | NH2 | ARG | E | 91 | −5.177 | 58.047 | 46.944 | 1.00 | 84.11 | N |
| ATOM | 9387 | C | ARG | E | 91 | −5.321 | 51.345 | 46.836 | 1.00 | 44.53 | C |
| ATOM | 9388 | O | ARG | E | 91 | −6.529 | 51.285 | 46.550 | 1.00 | 38.47 | O |
| ATOM | 9389 | N | ALA | E | 92 | −4.699 | 50.472 | 47.624 | 1.00 | 46.82 | N |
| ATOM | 9391 | CA | ALA | E | 92 | −5.442 | 49.475 | 48.373 | 1.00 | 54.07 | C |
| ATOM | 9393 | CB | ALA | E | 92 | −5.509 | 48.188 | 47.596 | 1.00 | 60.03 | C |
| ATOM | 9397 | C | ALA | E | 92 | −4.895 | 49.224 | 49.773 | 1.00 | 57.29 | C |
| ATOM | 9398 | O | ALA | E | 92 | −3.690 | 49.246 | 49.996 | 1.00 | 56.95 | O |
| ATOM | 9399 | N | GLU | E | 93 | −5.834 | 49.068 | 50.705 | 1.00 | 67.13 | N |
| ATOM | 9401 | CA | GLU | E | 93 | −5.668 | 48.475 | 52.031 | 1.00 | 69.06 | C |
| ATOM | 9403 | CB | GLU | E | 93 | −7.030 | 48.556 | 52.709 | 1.00 | 64.87 | C |
| ATOM | 9406 | CG | GLU | E | 93 | −6.998 | 49.295 | 54.030 | 1.00 | 85.50 | C |
| ATOM | 9409 | CD | GLU | E | 93 | −7.309 | 50.761 | 53.875 | 1.00 | 87.38 | C |
| ATOM | 9410 | OE1 | GLU | E | 93 | −8.341 | 51.078 | 53.251 | 1.00 | 101.21 | O |
| ATOM | 9411 | OE2 | GLU | E | 93 | −6.521 | 51.584 | 54.374 | 1.00 | 90.86 | O |
| ATOM | 9412 | C | GLU | E | 93 | −5.259 | 47.003 | 51.995 | 1.00 | 77.03 | C |
| ATOM | 9413 | O | GLU | E | 93 | −5.129 | 46.429 | 50.914 | 1.00 | 80.29 | O |
| ATOM | 9414 | N | ASP | E | 94 | −5.176 | 46.368 | 53.167 | 1.00 | 86.04 | N |
| ATOM | 9416 | CA | ASP | E | 94 | −4.497 | 45.075 | 53.315 | 1.00 | 89.28 | C |
| ATOM | 9418 | CB | ASP | E | 94 | −3.778 | 45.004 | 54.661 | 1.00 | 92.58 | C |
| ATOM | 9421 | CG | ASP | E | 94 | −2.537 | 45.890 | 54.697 | 1.00 | 100.64 | C |
| ATOM | 9422 | OD1 | ASP | E | 94 | −2.416 | 46.685 | 55.655 | 1.00 | 108.37 | O |
| ATOM | 9423 | OD2 | ASP | E | 94 | −1.661 | 45.896 | 53.795 | 1.00 | 94.19 | O |
| ATOM | 9424 | C | ASP | E | 94 | −5.342 | 43.823 | 53.094 | 1.00 | 87.19 | C |
| ATOM | 9425 | O | ASP | E | 94 | −4.883 | 42.856 | 52.485 | 1.00 | 87.17 | O |
| ATOM | 9426 | N | ASN | E | 95 | −6.579 | 43.839 | 53.571 | 1.00 | 91.85 | N |
| ATOM | 9428 | CA | ASN | E | 95 | −7.684 | 43.178 | 52.869 | 1.00 | 96.38 | C |
| ATOM | 9430 | CB | ASN | E | 95 | −8.907 | 43.086 | 53.797 | 1.00 | 100.23 | C |
| ATOM | 9433 | CG | ASN | E | 95 | −8.944 | 44.210 | 54.840 | 1.00 | 108.10 | C |
| ATOM | 9434 | OD1 | ASN | E | 95 | −9.494 | 45.290 | 54.601 | 1.00 | 95.85 | O |
| ATOM | 9435 | ND2 | ASN | E | 95 | −8.344 | 43.957 | 56.001 | 1.00 | 116.42 | N |
| ATOM | 9438 | C | ASN | E | 95 | −8.074 | 43.956 | 51.601 | 1.00 | 97.67 | C |
| ATOM | 9439 | O | ASN | E | 95 | −7.584 | 43.665 | 50.502 | 1.00 | 91.06 | O |
| ATOM | 9440 | N | ALA | E | 96 | −8.856 | 45.024 | 51.809 | 1.00 | 97.64 | N |
| ATOM | 9442 | CA | ALA | E | 96 | −9.995 | 45.433 | 50.966 | 1.00 | 87.03 | C |
| ATOM | 9444 | CB | ALA | E | 96 | −9.819 | 46.872 | 50.417 | 1.00 | 86.68 | C |
| ATOM | 9448 | C | ALA | E | 96 | −10.312 | 44.445 | 49.854 | 1.00 | 78.33 | C |
| ATOM | 9449 | O | ALA | E | 96 | −9.457 | 43.755 | 49.301 | 1.00 | 62.28 | O |
| ATOM | 9450 | N | ASP | E | 97 | −11.599 | 44.346 | 49.585 | 1.00 | 76.82 | N |
| ATOM | 9452 | CA | ASP | E | 97 | −12.071 | 43.557 | 48.474 | 1.00 | 77.82 | C |
| ATOM | 9454 | CB | ASP | E | 97 | −13.321 | 42.805 | 48.919 | 1.00 | 85.42 | C |
| ATOM | 9457 | CG | ASP | E | 97 | −13.976 | 43.460 | 50.114 | 1.00 | 93.73 | C |
| ATOM | 9458 | OD1 | ASP | E | 97 | −13.584 | 43.137 | 51.258 | 1.00 | 109.11 | O |
| ATOM | 9459 | OD2 | ASP | E | 97 | −14.840 | 44.355 | 49.997 | 1.00 | 111.48 | O |
| ATOM | 9460 | C | ASP | E | 97 | −12.370 | 44.602 | 47.416 | 1.00 | 65.19 | C |
| ATOM | 9461 | O | ASP | E | 97 | −13.164 | 44.416 | 46.512 | 1.00 | 67.62 | O |
| ATOM | 9462 | N | THR | E | 98 | −11.685 | 45.721 | 47.535 | 1.00 | 57.37 | N |
| ATOM | 9464 | CA | THR | E | 98 | −11.864 | 46.804 | 46.590 | 1.00 | 58.44 | C |
| ATOM | 9466 | CB | THR | E | 98 | −12.772 | 47.881 | 47.229 | 1.00 | 48.76 | C |
| ATOM | 9468 | OG1 | THR | E | 98 | −12.062 | 49.125 | 47.348 | 1.00 | 66.66 | O |
| ATOM | 9470 | CG2 | THR | E | 98 | −13.081 | 47.520 | 48.689 | 1.00 | 77.74 | C |
| ATOM | 9474 | C | THR | E | 98 | −10.483 | 47.343 | 46.203 | 1.00 | 48.03 | C |
| ATOM | 9475 | O | THR | E | 98 | −9.539 | 47.229 | 46.979 | 1.00 | 45.63 | O |
| ATOM | 9476 | N | LEU | E | 99 | −10.363 | 47.888 | 44.995 | 1.00 | 44.66 | N |
| ATOM | 9478 | CA | LEU | E | 99 | −9.197 | 48.678 | 44.600 | 1.00 | 43.97 | C |
| ATOM | 9480 | CB | LEU | E | 99 | −8.528 | 48.069 | 43.359 | 1.00 | 47.82 | C |
| ATOM | 9483 | CG | LEU | E | 99 | −7.522 | 48.951 | 42.630 | 1.00 | 48.58 | C |
| ATOM | 9485 | CD1 | LEU | E | 99 | −6.412 | 49.456 | 43.548 | 1.00 | 58.09 | C |
| ATOM | 9489 | CD2 | LEU | E | 99 | −6.931 | 48.205 | 41.470 | 1.00 | 47.58 | C |
| ATOM | 9493 | C | LEU | E | 99 | −9.648 | 50.080 | 44.237 | 1.00 | 50.23 | C |
| ATOM | 9494 | O | LEU | E | 99 | −10.758 | 50.297 | 43.752 | 1.00 | 54.26 | O |
| ATOM | 9495 | N | ALA | E | 100 | −8.770 | 51.043 | 44.466 | 1.00 | 51.83 | N |
| ATOM | 9497 | CA | ALA | E | 100 | −9.135 | 52.433 | 44.289 | 1.00 | 46.91 | C |
| ATOM | 9499 | CB | ALA | E | 100 | −9.016 | 53.151 | 45.618 | 1.00 | 44.48 | C |
| ATOM | 9503 | C | ALA | E | 100 | −8.247 | 53.085 | 43.226 | 1.00 | 49.71 | C |
| ATOM | 9504 | O | ALA | E | 100 | −7.015 | 53.013 | 43.283 | 1.00 | 50.17 | O |
| ATOM | 9505 | N | LEU | E | 101 | −8.882 | 53.673 | 42.217 | 1.00 | 50.19 | N |
| ATOM | 9507 | CA | LEU | E | 101 | −8.132 | 54.349 | 41.166 | 1.00 | 42.02 | C |
| ATOM | 9509 | CB | LEU | E | 101 | −8.435 | 53.759 | 39.796 | 1.00 | 25.56 | C |
| ATOM | 9512 | CG | LEU | E | 101 | −8.314 | 52.235 | 39.675 | 1.00 | 31.74 | C |
| ATOM | 9514 | CD1 | LEU | E | 101 | −9.549 | 51.484 | 40.183 | 1.00 | 40.00 | C |
| ATOM | 9518 | CD2 | LEU | E | 101 | −8.012 | 51.801 | 38.237 | 1.00 | 35.50 | C |
| ATOM | 9522 | C | LEU | E | 101 | −8.526 | 55.800 | 41.218 | 1.00 | 43.46 | C |
| ATOM | 9523 | O | LEU | E | 101 | −9.675 | 56.144 | 41.506 | 1.00 | 48.57 | O |
| ATOM | 9524 | N | VAL | E | 102 | −7.531 | 56.648 | 41.038 | 1.00 | 45.29 | N |
| ATOM | 9526 | CA | VAL | E | 102 | −7.730 | 58.073 | 41.191 | 1.00 | 48.82 | C |
| ATOM | 9528 | CB | VAL | E | 102 | −7.165 | 58.583 | 42.529 | 1.00 | 52.07 | C |
| ATOM | 9530 | CG1 | VAL | E | 102 | −6.930 | 60.097 | 42.504 | 1.00 | 65.38 | C |
| ATOM | 9534 | CG2 | VAL | E | 102 | −8.104 | 58.194 | 43.644 | 1.00 | 48.86 | C |
| ATOM | 9538 | C | VAL | E | 102 | −7.031 | 58.692 | 40.000 | 1.00 | 49.40 | C |
| ATOM | 9539 | O | VAL | E | 102 | −5.867 | 58.389 | 39.728 | 1.00 | 46.02 | O |
| ATOM | 9540 | N | PHE | E | 103 | −7.815 | 59.464 | 39.249 | 1.00 | 48.39 | N |
| ATOM | 9542 | CA | PHE | E | 103 | −7.363 | 60.130 | 38.041 | 1.00 | 44.57 | C |
| ATOM | 9544 | CB | PHE | E | 103 | −8.345 | 59.826 | 36.920 | 1.00 | 39.32 | C |
| ATOM | 9547 | CG | PHE | E | 103 | −8.473 | 58.383 | 36.620 | 1.00 | 28.87 | C |
| ATOM | 9548 | CD1 | PHE | E | 103 | −7.415 | 57.521 | 36.855 | 1.00 | 20.77 | C |
| ATOM | 9550 | CE1 | PHE | E | 103 | −7.544 | 56.159 | 36.613 | 1.00 | 46.82 | C |
| ATOM | 9552 | CZ | PHE | E | 103 | −8.734 | 55.665 | 36.114 | 1.00 | 56.15 | C |
| ATOM | 9554 | CE2 | PHE | E | 103 | −9.794 | 56.544 | 35.859 | 1.00 | 58.59 | C |
| ATOM | 9556 | CD2 | PHE | E | 103 | −9.658 | 57.885 | 36.120 | 1.00 | 26.86 | C |
| ATOM | 9558 | C | PHE | E | 103 | −7.300 | 61.644 | 38.239 | 1.00 | 48.16 | C |
| ATOM | 9559 | O | PHE | E | 103 | −8.342 | 62.279 | 38.387 | 1.00 | 52.48 | O |
| ATOM | 9560 | N | GLU | E | 104 | −6.098 | 62.213 | 38.202 | 1.00 | 43.31 | N |
| ATOM | 9562 | CA | GLU | E | 104 | −5.915 | 63.630 | 38.423 | 1.00 | 49.71 | C |
| ATOM | 9564 | CB | GLU | E | 104 | −4.709 | 63.900 | 39.343 | 1.00 | 58.43 | C |
| ATOM | 9567 | CG | GLU | E | 104 | −4.956 | 63.718 | 40.843 | 1.00 | 74.81 | C |
| ATOM | 9570 | CD | GLU | E | 104 | −3.733 | 63.257 | 41.639 | 1.00 | 81.23 | C |
| ATOM | 9571 | OE1 | GLU | E | 104 | −3.201 | 62.141 | 41.387 | 1.00 | 79.88 | O |
| ATOM | 9572 | OE2 | GLU | E | 104 | −3.349 | 63.999 | 42.571 | 1.00 | 88.25 | O |
| ATOM | 9573 | C | GLU | E | 104 | −5.613 | 64.221 | 37.071 | 1.00 | 55.93 | C |
| ATOM | 9574 | O | GLU | E | 104 | −4.596 | 63.902 | 36.467 | 1.00 | 53.10 | O |
| ATOM | 9575 | N | ALA | E | 105 | −6.462 | 65.132 | 36.619 | 1.00 | 64.66 | N |
| ATOM | 9577 | CA | ALA | E | 105 | −6.218 | 65.788 | 35.344 | 1.00 | 67.90 | C |
| ATOM | 9579 | CB | ALA | E | 105 | −7.514 | 66.342 | 34.769 | 1.00 | 69.90 | C |
| ATOM | 9583 | C | ALA | E | 105 | −5.214 | 66.896 | 35.567 | 1.00 | 69.38 | C |
| ATOM | 9584 | O | ALA | E | 105 | −5.141 | 67.461 | 36.646 | 1.00 | 73.20 | O |
| ATOM | 9585 | N | PRO | E | 106 | −4.402 | 67.173 | 34.564 | 1.00 | 79.19 | N |
| ATOM | 9586 | CA | PRO | E | 106 | −3.010 | 67.524 | 34.822 | 1.00 | 89.68 | C |
| ATOM | 9588 | CB | PRO | E | 106 | −2.273 | 66.766 | 33.715 | 1.00 | 89.39 | C |
| ATOM | 9591 | CG | PRO | E | 106 | −3.373 | 66.550 | 32.616 | 1.00 | 86.02 | C |
| ATOM | 9594 | CD | PRO | E | 106 | −4.683 | 67.132 | 33.119 | 1.00 | 80.04 | C |
| ATOM | 9597 | C | PRO | E | 106 | −2.838 | 69.038 | 34.693 | 1.00 | 95.81 | C |
| ATOM | 9598 | O | PRO | E | 106 | −2.460 | 69.678 | 35.674 | 1.00 | 96.63 | O |
| ATOM | 9599 | N | ASN | E | 107 | −3.131 | 69.585 | 33.512 | 1.00 | 102.65 | N |
| ATOM | 9601 | CA | ASN | E | 107 | −3.740 | 70.915 | 33.378 | 1.00 | 106.21 | C |
| ATOM | 9603 | CB | ASN | E | 107 | −3.507 | 71.505 | 31.970 | 1.00 | 105.37 | C |
| ATOM | 9606 | CG | ASN | E | 107 | −2.032 | 71.696 | 31.642 | 1.00 | 105.73 | C |
| ATOM | 9607 | OD1 | ASN | E | 107 | −1.662 | 71.853 | 30.475 | 1.00 | 108.04 | O |
| ATOM | 9608 | ND2 | ASN | E | 107 | −1.180 | 71.655 | 32.665 | 1.00 | 93.17 | N |
| ATOM | 9611 | C | ASN | E | 107 | −5.242 | 70.890 | 33.710 | 1.00 | 102.72 | C |
| ATOM | 9612 | O | ASN | E | 107 | −6.098 | 70.822 | 32.818 | 1.00 | 105.15 | O |
| ATOM | 9613 | N | GLN | E | 108 | −5.557 | 70.973 | 35.000 | 1.00 | 93.97 | N |
| ATOM | 9615 | CA | GLN | E | 108 | −6.939 | 71.130 | 35.411 | 1.00 | 84.25 | C |
| ATOM | 9617 | CB | GLN | E | 108 | −7.833 | 70.192 | 34.596 | 1.00 | 76.95 | C |
| ATOM | 9620 | CG | GLN | E | 108 | −8.815 | 70.905 | 33.694 | 1.00 | 77.77 | C |
| ATOM | 9623 | CD | GLN | E | 108 | −9.817 | 69.959 | 33.049 | 1.00 | 86.24 | C |
| ATOM | 9624 | OE1 | GLN | E | 108 | −9.427 | 68.916 | 32.516 | 1.00 | 86.26 | O |
| ATOM | 9625 | NE2 | GLN | E | 108 | −11.104 | 70.319 | 33.090 | 1.00 | 84.64 | N |
| ATOM | 9628 | C | GLN | E | 108 | −7.194 | 70.900 | 36.898 | 1.00 | 83.90 | C |
| ATOM | 9629 | O | GLN | E | 108 | −6.290 | 70.818 | 37.748 | 1.00 | 77.78 | O |
| ATOM | 9630 | N | GLU | E | 109 | −8.491 | 70.801 | 37.150 | 1.00 | 81.33 | N |
| ATOM | 9632 | CA | GLU | E | 109 | −9.096 | 70.854 | 38.461 | 1.00 | 80.00 | C |
| ATOM | 9634 | CB | GLU | E | 109 | −9.752 | 72.220 | 38.639 | 1.00 | 86.34 | C |
| ATOM | 9637 | CD | GLU | E | 109 | −11.185 | 72.233 | 38.118 | 1.00 | 94.70 | C |
| ATOM | 9640 | CD | GLU | E | 109 | −11.295 | 72.666 | 36.665 | 1.00 | 116.42 | C |
| ATOM | 9641 | OE1 | GLU | E | 109 | −10.424 | 73.434 | 36.203 | 1.00 | 126.92 | O |
| ATOM | 9642 | OE2 | GLU | E | 109 | −12.262 | 72.258 | 35.981 | 1.00 | 122.09 | O |
| ATOM | 9643 | C | GLU | E | 109 | −10.199 | 69.808 | 38.395 | 1.00 | 69.28 | C |
| ATOM | 9644 | O | GLU | E | 109 | −11.208 | 69.889 | 39.084 | 1.00 | 74.29 | O |
| ATOM | 9645 | N | LYS | E | 110 | −10.038 | 68.860 | 37.487 | 1.00 | 60.31 | N |
| ATOM | 9647 | CA | LYS | E | 110 | −10.886 | 67.682 | 37.446 | 1.00 | 54.29 | C |
| ATOM | 9649 | CB | LYS | E | 110 | −11.195 | 67.324 | 35.981 | 1.00 | 54.85 | C |
| ATOM | 9652 | CG | LYS | E | 110 | −11.716 | 65.887 | 35.684 | 1.00 | 71.18 | C |
| ATOM | 9655 | CD | LYS | E | 110 | −12.488 | 65.803 | 34.348 | 1.00 | 73.55 | C |
| ATOM | 9658 | CE | LYS | E | 110 | −12.242 | 64.545 | 33.524 | 1.00 | 52.20 | C |
| ATOM | 9661 | NZ | LYS | E | 110 | −13.527 | 63.806 | 33.393 | 1.00 | 49.38 | N |
| ATOM | 9665 | C | LYS | E | 110 | −10.118 | 66.581 | 38.192 | 1.00 | 53.32 | C |
| ATOM | 9666 | O | LYS | E | 110 | −8.932 | 66.313 | 37.929 | 1.00 | 57.48 | O |
| ATOM | 9667 | N | VAL | E | 111 | −10.766 | 66.015 | 39.203 | 1.00 | 47.08 | N |
| ATOM | 9669 | CA | VAL | E | 111 | −10.274 | 64.796 | 39.828 | 1.00 | 46.57 | C |
| ATOM | 9671 | CB | VAL | E | 111 | −9.769 | 65.042 | 41.264 | 1.00 | 46.42 | C |
| ATOM | 9673 | CG1 | VAL | E | 111 | −9.345 | 63.725 | 41.876 | 1.00 | 45.34 | C |
| ATOM | 9677 | CG2 | VAL | E | 111 | −8.629 | 66.059 | 41.280 | 1.00 | 46.34 | C |
| ATOM | 9681 | C | VAL | E | 111 | −11.389 | 63.744 | 39.852 | 1.00 | 53.33 | C |
| ATOM | 9682 | O | VAL | E | 111 | −12.549 | 64.004 | 40.197 | 1.00 | 53.34 | O |
| ATOM | 9683 | N | SER | E | 112 | −11.041 | 62.560 | 39.379 | 1.00 | 55.78 | N |
| ATOM | 9685 | CA | SER | E | 112 | −12.007 | 61.485 | 39.278 | 1.00 | 57.91 | C |
| ATOM | 9687 | CB | SER | E | 112 | −12.032 | 60.907 | 37.854 | 1.00 | 60.25 | C |
| ATOM | 9690 | OG | SER | E | 112 | −12.863 | 61.651 | 36.971 | 1.00 | 57.56 | O |
| ATOM | 9692 | C | SER | E | 112 | −11.478 | 60.451 | 40.251 | 1.00 | 54.96 | C |
| ATOM | 9693 | O | SER | E | 112 | −10.263 | 60.248 | 40.366 | 1.00 | 58.00 | O |
| ATOM | 9694 | N | ASP | E | 113 | −12.380 | 59.785 | 40.951 | 1.00 | 46.74 | N |
| ATOM | 9696 | CA | ASP | E | 113 | −11.935 | 58.824 | 41.936 | 1.00 | 46.70 | C |
| ATOM | 9698 | CB | ASP | E | 113 | −11.878 | 59.516 | 43.309 | 1.00 | 49.94 | C |
| ATOM | 9701 | CG | ASP | E | 113 | −12.606 | 58.748 | 44.366 | 1.00 | 72.65 | C |
| ATOM | 9702 | OD1 | ASP | E | 113 | −13.735 | 59.148 | 44.730 | 1.00 | 91.44 | O |
| ATOM | 9703 | OD2 | ASP | E | 113 | −12.129 | 57.701 | 44.853 | 1.00 | 110.28 | O |
| ATOM | 9704 | C | ASP | E | 113 | −12.861 | 57.608 | 41.849 | 1.00 | 40.05 | C |
| ATOM | 9705 | O | ASP | E | 113 | −14.068 | 57.740 | 41.844 | 1.00 | 40.17 | O |
| ATOM | 9706 | N | TYR | E | 114 | −12.317 | 56.436 | 41.564 | 1.00 | 43.78 | N |
| ATOM | 9708 | CA | TYR | E | 114 | −13.166 | 55.282 | 41.259 | 1.00 | 45.27 | C |
| ATOM | 9710 | CB | TYR | E | 114 | −12.903 | 54.756 | 39.843 | 1.00 | 44.67 | C |
| ATOM | 9713 | CG | TYR | E | 114 | −13.381 | 55.668 | 38.737 | 1.00 | 52.26 | C |
| ATOM | 9714 | CD1 | TYR | E | 114 | −12.526 | 56.569 | 38.111 | 1.00 | 50.29 | C |
| ATOM | 9716 | CE1 | TYR | E | 114 | −12.996 | 57.458 | 37.154 | 1.00 | 52.30 | C |
| ATOM | 9718 | CZ | TYR | E | 114 | −14.311 | 57.377 | 36.777 | 1.00 | 52.78 | C |
| ATOM | 9719 | OH | TYR | E | 114 | −14.809 | 58.142 | 35.770 | 1.00 | 56.16 | O |
| ATOM | 9721 | CE2 | TYR | E | 114 | −15.160 | 56.494 | 37.371 | 1.00 | 52.30 | C |
| ATOM | 9723 | CD2 | TYR | E | 114 | −14.701 | 55.646 | 38.341 | 1.00 | 59.10 | C |
| ATOM | 9725 | C | TYR | E | 114 | −12.845 | 54.184 | 42.270 | 1.00 | 52.69 | C |
| ATOM | 9726 | O | TYR | E | 114 | −11.784 | 54.191 | 42.913 | 1.00 | 56.85 | O |
| ATOM | 9727 | N | GLU | E | 115 | −13.751 | 53.221 | 42.375 | 1.00 | 47.80 | N |
| ATOM | 9729 | CA | GLU | E | 115 | −13.495 | 52.037 | 43.161 | 1.00 | 46.17 | C |
| ATOM | 9731 | CB | GLU | E | 115 | −14.157 | 52.256 | 44.503 | 1.00 | 57.10 | C |
| ATOM | 9734 | CG | GLU | E | 115 | −14.544 | 51.012 | 45.288 | 1.00 | 65.95 | C |
| ATOM | 9737 | CD | GLU | E | 115 | −14.859 | 51.390 | 46.717 | 1.00 | 65.09 | C |
| ATOM | 9738 | OE1 | GLU | E | 115 | −13.888 | 51.552 | 47.486 | 1.00 | 68.50 | O |
| ATOM | 9739 | OE2 | GLU | E | 115 | −16.042 | 51.647 | 47.010 | 1.00 | 63.89 | O |
| ATOM | 9740 | C | GLU | E | 115 | −14.012 | 50.747 | 42.509 | 1.00 | 47.86 | C |
| ATOM | 9741 | O | GLU | E | 115 | −15.150 | 50.643 | 42.073 | 1.00 | 42.84 | O |
| ATOM | 9742 | N | MET | E | 116 | −13.159 | 49.737 | 42.487 | 1.00 | 53.18 | N |
| ATOM | 9744 | CA | MET | E | 116 | −13.335 | 48.573 | 41.630 | 1.00 | 51.72 | C |
| ATOM | 9746 | CB | MET | E | 116 | −12.068 | 48.297 | 40.826 | 1.00 | 50.33 | C |
| ATOM | 9749 | CG | MET | E | 116 | −12.304 | 47.640 | 39.524 | 1.00 | 43.17 | C |
| ATOM | 9752 | SD | MET | E | 116 | −10.752 | 46.930 | 38.963 | 1.00 | 60.88 | S |
| ATOM | 9753 | CE | MET | E | 116 | −11.297 | 45.258 | 38.513 | 1.00 | 86.65 | C |
| ATOM | 9757 | C | MET | E | 116 | −13.521 | 47.461 | 42.636 | 1.00 | 55.55 | C |
| ATOM | 9758 | O | MET | E | 116 | −12.757 | 47.361 | 43.600 | 1.00 | 49.09 | O |
| ATOM | 9759 | N | LYS | E | 117 | −14.607 | 46.720 | 42.466 | 1.00 | 54.78 | N |
| ATOM | 9761 | CA | LYS | E | 117 | −14.744 | 45.429 | 43.083 | 1.00 | 57.89 | C |
| ATOM | 9763 | CB | LYS | E | 117 | −16.142 | 44.866 | 42.817 | 1.00 | 64.80 | C |
| ATOM | 9766 | CG | LYS | E | 117 | −17.284 | 45.557 | 43.567 | 1.00 | 70.42 | C |
| ATOM | 9769 | CD | LYS | E | 117 | −18.632 | 45.378 | 42.847 | 1.00 | 69.63 | C |
| ATOM | 9772 | CE | LYS | E | 117 | −19.817 | 45.844 | 43.693 | 1.00 | 68.22 | C |
| ATOM | 9775 | NZ | LYS | E | 117 | −21.098 | 45.895 | 42.914 | 1.00 | 70.09 | N |
| ATOM | 9779 | C | LYS | E | 117 | −13.696 | 44.485 | 42.505 | 1.00 | 58.97 | C |
| ATOM | 9780 | O | LYS | E | 117 | −13.349 | 44.522 | 41.314 | 1.00 | 60.04 | O |
| ATOM | 9781 | N | LEU | E | 118 | −13.208 | 43.638 | 43.402 | 1.00 | 61.83 | N |
| ATOM | 9783 | CA | LEU | E | 118 | −12.143 | 42.677 | 43.141 | 1.00 | 62.85 | C |
| ATOM | 9785 | CB | LEU | E | 118 | −11.057 | 42.820 | 44.205 | 1.00 | 55.54 | C |
| ATOM | 9788 | CG | LEU | E | 118 | −10.313 | 44.135 | 43.987 | 1.00 | 58.40 | C |
| ATOM | 9790 | CD1 | LEU | E | 118 | −9.501 | 44.584 | 45.157 | 1.00 | 55.72 | C |
| ATOM | 9794 | CD2 | LEU | E | 118 | −9.381 | 43.937 | 42.809 | 1.00 | 62.61 | C |
| ATOM | 9798 | C | LEU | E | 118 | −12.718 | 41.266 | 43.165 | 1.00 | 64.99 | C |
| ATOM | 9799 | O | LEU | E | 118 | −13.584 | 40.979 | 43.971 | 1.00 | 76.29 | O |
| ATOM | 9800 | N | MET | E | 119 | −12.303 | 40.401 | 42.249 | 1.00 | 65.66 | N |
| ATOM | 9802 | CA | MET | E | 119 | −12.788 | 39.037 | 42.256 | 1.00 | 60.24 | C |
| ATOM | 9804 | CB | MET | E | 119 | −12.538 | 38.419 | 40.894 | 1.00 | 63.86 | C |
| ATOM | 9807 | CG | MET | E | 119 | −13.471 | 38.965 | 39.877 | 1.00 | 59.95 | C |
| ATOM | 9810 | SD | MET | E | 119 | −13.377 | 37.084 | 38.493 | 1.00 | 75.35 | S |
| ATOM | 9811 | CE | MET | E | 119 | −15.003 | 38.479 | 37.645 | 1.00 | 75.24 | C |
| ATOM | 9815 | C | MET | E | 119 | −11.974 | 38.307 | 43.290 | 1.00 | 57.79 | C |
| ATOM | 9816 | O | MET | E | 119 | −10.860 | 38.708 | 43.579 | 1.00 | 57.50 | O |
| ATOM | 9817 | N | ASP | E | 120 | −12.521 | 37.236 | 43.842 | 1.00 | 65.26 | N |
| ATOM | 9819 | CA | ASP | E | 120 | −11.690 | 36.165 | 44.396 | 1.00 | 70.68 | C |
| ATOM | 9821 | CB | ASP | E | 120 | −12.218 | 35.629 | 45.739 | 1.00 | 72.62 | C |
| ATOM | 9824 | CG | ASP | E | 120 | −11.174 | 35.745 | 46.835 | 1.00 | 83.14 | C |
| ATOM | 9825 | OD1 | ASP | E | 120 | −10.104 | 35.111 | 46.700 | 1.00 | 86.61 | O |
| ATOM | 9826 | OD2 | ASP | E | 120 | −11.267 | 36.538 | 47.793 | 1.00 | 95.53 | O |
| ATOM | 9827 | C | ASP | E | 120 | −11.446 | 35.034 | 43.414 | 1.00 | 62.53 | C |
| ATOM | 9828 | O | ASP | E | 120 | −12.346 | 34.540 | 42.766 | 1.00 | 59.92 | O |
| ATOM | 9829 | N | LEU | E | 121 | −10.188 | 34.667 | 43.269 | 1.00 | 64.96 | N |
| ATOM | 9831 | CA | LEU | E | 121 | −9.796 | 33.614 | 42.337 | 1.00 | 63.96 | C |
| ATOM | 9833 | CB | LEU | E | 121 | −9.915 | 34.018 | 40.868 | 1.00 | 57.93 | C |
| ATOM | 9836 | CG | LEU | E | 121 | −9.691 | 35.465 | 40.452 | 1.00 | 66.94 | C |
| ATOM | 9838 | CD1 | LEU | E | 121 | −8.287 | 35.680 | 39.902 | 1.00 | 48.47 | C |
| ATOM | 9842 | CD2 | LEU | E | 121 | −10.773 | 35.879 | 39.449 | 1.00 | 78.46 | C |
| ATOM | 9846 | C | LEU | E | 121 | −8.361 | 33.265 | 42.611 | 1.00 | 60.25 | C |
| ATOM | 9847 | O | LEU | E | 121 | −7.538 | 34.162 | 42.774 | 1.00 | 71.07 | O |
| ATOM | 9848 | N | ASP | E | 122 | −8.072 | 31.977 | 42.714 | 1.00 | 61.43 | N |
| ATOM | 9850 | CA | ASP | E | 122 | −6.708 | 31.583 | 42.999 | 1.00 | 76.67 | C |
| ATOM | 9852 | CB | ASP | E | 122 | −6.557 | 30.770 | 44.304 | 1.00 | 84.70 | C |
| ATOM | 9855 | CG | ASP | E | 122 | −7.578 | 29.624 | 44.434 | 1.00 | 94.15 | C |
| ATOM | 9856 | OD1 | ASP | E | 122 | −7.308 | 28.535 | 43.877 | 1.00 | 98.23 | O |
| ATOM | 9857 | OD2 | ASP | E | 122 | −8.636 | 29.696 | 45.112 | 1.00 | 87.31 | O |
| ATOM | 9858 | C | ASP | E | 122 | −6.243 | 30.838 | 41.766 | 1.00 | 77.09 | C |
| ATOM | 9859 | O | ASP | E | 122 | −7.066 | 30.379 | 40.961 | 1.00 | 75.66 | O |
| ATOM | 9860 | N | VAL | E | 123 | −4.927 | 30.851 | 41.590 | 1.00 | 75.27 | N |
| ATOM | 9862 | CA | VAL | E | 123 | −4.284 | 30.402 | 40.370 | 1.00 | 79.27 | C |
| ATOM | 9864 | CB | VAL | E | 123 | −4.345 | 31.468 | 39.261 | 1.00 | 83.96 | C |
| ATOM | 9866 | CG1 | VAL | E | 123 | −5.385 | 31.083 | 38.183 | 1.00 | 94.81 | C |
| ATOM | 9870 | CG2 | VAL | E | 123 | −4.621 | 32.842 | 39.872 | 1.00 | 72.21 | C |
| ATOM | 9874 | C | VAL | E | 123 | −2.867 | 30.343 | 40.840 | 1.00 | 77.24 | C |
| ATOM | 9875 | O | VAL | E | 123 | −2.484 | 31.238 | 41.594 | 1.00 | 78.40 | O |
| ATOM | 9876 | N | GLU | E | 124 | −2.125 | 29.297 | 40.471 | 1.00 | 75.28 | N |
| ATOM | 9878 | CA | GLU | E | 124 | −0.717 | 29.214 | 40.871 | 1.00 | 74.97 | C |
| ATOM | 9880 | CB | GLU | E | 124 | −0.266 | 27.857 | 41.461 | 1.00 | 79.22 | C |
| ATOM | 9883 | CG | GLU | E | 124 | −1.282 | 26.719 | 41.469 | 1.00 | 95.44 | C |
| ATOM | 9886 | CD | GLU | E | 124 | −1.287 | 25.917 | 40.172 | 1.00 | 102.45 | C |
| ATOM | 9887 | OE1 | GLU | E | 124 | −1.751 | 26.456 | 39.141 | 1.00 | 107.06 | O |
| ATOM | 9888 | OE2 | GLU | E | 124 | −0.844 | 24.745 | 40.185 | 1.00 | 94.78 | O |
| ATOM | 9889 | C | GLU | E | 124 | 0.166 | 29.648 | 39.714 | 1.00 | 62.89 | C |
| ATOM | 9890 | O | GLU | E | 124 | −0.197 | 29.493 | 38.565 | 1.00 | 51.13 | O |
| ATOM | 9891 | N | GLN | E | 125 | 1.266 | 30.302 | 40.060 | 1.00 | 60.03 | N |
| ATOM | 9893 | CA | GLN | E | 125 | 2.281 | 30.685 | 39.106 | 1.00 | 66.64 | C |
| ATOM | 9895 | CB | GLN | E | 125 | 3.341 | 31.596 | 39.758 | 1.00 | 69.73 | C |
| ATOM | 9898 | CG | GLN | E | 125 | 2.767 | 32.735 | 40.625 | 1.00 | 96.20 | C |
| ATOM | 9901 | CD | GLN | E | 125 | 2.029 | 33.836 | 39.839 | 1.00 | 111.49 | C |
| ATOM | 9902 | OE1 | GLN | E | 125 | 2.626 | 34.869 | 39.494 | 1.00 | 102.88 | O |
| ATOM | 9903 | NE2 | GLN | E | 125 | 0.722 | 33.641 | 39.610 | 1.00 | 91.28 | N |
| ATOM | 9906 | C | GLN | E | 125 | 2.907 | 29.474 | 38.395 | 1.00 | 65.19 | C |
| ATOM | 9907 | O | GLN | E | 125 | 3.183 | 28.420 | 38.980 | 1.00 | 59.41 | O |
| ATOM | 9908 | N | LEU | E | 126 | 3.085 | 29.635 | 37.091 | 1.00 | 59.56 | N |
| ATOM | 9910 | CA | LEU | E | 126 | 3.958 | 28.754 | 36.375 | 1.00 | 60.68 | C |
| ATOM | 9912 | CB | LEU | E | 126 | 3.535 | 28.690 | 34.910 | 1.00 | 65.23 | C |
| ATOM | 9915 | CG | LEU | E | 126 | 2.174 | 28.036 | 34.695 | 1.00 | 54.92 | C |
| ATOM | 9917 | CD1 | LEU | E | 126 | 1.805 | 27.950 | 33.232 | 1.00 | 40.92 | C |
| ATOM | 9921 | CD2 | LEU | E | 126 | 2.274 | 26.659 | 35.283 | 1.00 | 79.93 | C |
| ATOM | 9925 | C | LEU | E | 126 | 5.342 | 29.346 | 36.524 | 1.00 | 58.95 | C |
| ATOM | 9926 | O | LEU | E | 126 | 5.486 | 30.554 | 36.639 | 1.00 | 54.90 | O |
| ATOM | 9927 | N | GLY | E | 127 | 6.337 | 28.462 | 36.527 | 1.00 | 58.54 | N |
| ATOM | 9929 | CA | GLY | E | 127 | 7.740 | 28.804 | 36.638 | 1.00 | 49.76 | C |
| ATOM | 9932 | C | GLY | E | 127 | 8.367 | 28.819 | 35.267 | 1.00 | 49.94 | C |
| ATOM | 9933 | O | GLY | E | 127 | 8.228 | 27.865 | 34.512 | 1.00 | 61.48 | O |
| ATOM | 9934 | N | ILE | E | 128 | 9.019 | 29.927 | 34.942 | 1.00 | 53.52 | N |
| ATOM | 9936 | CA | ILE | E | 128 | 9.540 | 30.179 | 33.614 | 1.00 | 52.20 | C |
| ATOM | 9938 | CB | ILE | E | 128 | 9.031 | 31.517 | 33.095 | 1.00 | 52.65 | C |
| ATOM | 9940 | CG1 | ILE | E | 128 | 7.507 | 31.490 | 33.088 | 1.00 | 57.02 | C |
| ATOM | 9943 | CD1 | ILE | E | 128 | 6.871 | 32.837 | 32.901 | 1.00 | 64.24 | C |
| ATOM | 9947 | CG2 | ILE | E | 128 | 9.593 | 31.787 | 31.690 | 1.00 | 55.70 | C |
| ATOM | 9951 | C | ILE | E | 128 | 11.049 | 30.212 | 33.689 | 1.00 | 57.61 | C |
| ATOM | 9952 | O | ILE | E | 128 | 11.665 | 31.232 | 34.015 | 1.00 | 54.06 | O |
| ATOM | 9953 | N | PRO | E | 129 | 11.637 | 29.061 | 33.407 | 1.00 | 65.30 | N |
| ATOM | 9954 | CA | PRO | E | 129 | 13.082 | 28.911 | 33.472 | 1.00 | 69.11 | C |
| ATOM | 9956 | CB | PRO | E | 129 | 13.326 | 27.629 | 32.674 | 1.00 | 77.39 | C |
| ATOM | 9959 | CG | PRO | E | 129 | 12.022 | 27.345 | 31.943 | 1.00 | 79.26 | C |
| ATOM | 9962 | CD | PRO | E | 129 | 10.987 | 27.839 | 32.909 | 1.00 | 67.33 | C |
| ATOM | 9965 | C | PRO | E | 129 | 13.731 | 30.100 | 32.790 | 1.00 | 73.00 | C |
| ATOM | 9966 | O | PRO | E | 129 | 13.302 | 30.545 | 31.721 | 1.00 | 72.36 | O |
| ATOM | 9967 | N | GLU | E | 130 | 14.769 | 30.619 | 33.429 | 1.00 | 78.03 | N |
| ATOM | 9969 | CA | GLU | E | 130 | 15.687 | 31.516 | 32.754 | 1.00 | 82.65 | C |
| ATOM | 9971 | CB | GLU | E | 130 | 16.801 | 31.969 | 33.691 | 1.00 | 86.66 | C |
| ATOM | 9974 | CG | GLU | E | 130 | 17.191 | 33.420 | 33.478 | 1.00 | 104.12 | C |
| ATOM | 9977 | CD | GLU | E | 130 | 17.684 | 34.044 | 34.764 | 1.00 | 118.68 | C |
| ATOM | 9978 | OE1 | GLU | E | 130 | 18.878 | 34.433 | 34.818 | 1.00 | 120.22 | O |
| ATOM | 9979 | OE2 | GLU | E | 130 | 16.863 | 34.108 | 35.710 | 1.00 | 103.39 | O |
| ATOM | 9980 | C | GLU | E | 130 | 16.254 | 30.665 | 31.652 | 1.00 | 74.03 | C |
| ATOM | 9981 | O | GLU | E | 130 | 16.306 | 29.452 | 31.775 | 1.00 | 74.86 | O |
| ATOM | 9982 | N | GLN | E | 131 | 16.632 | 31.281 | 30.551 | 1.00 | 70.76 | N |
| ATOM | 9984 | CA | GLN | E | 131 | 17.053 | 30.471 | 29.424 | 1.00 | 69.20 | C |
| ATOM | 9986 | CB | GLN | E | 131 | 15.947 | 29.512 | 28.947 | 1.00 | 66.48 | C |
| ATOM | 9989 | CG | GLN | E | 131 | 14.991 | 29.966 | 27.842 | 1.00 | 71.13 | C |
| ATOM | 9992 | CD | GLN | E | 131 | 14.282 | 28.771 | 27.184 | 1.00 | 79.72 | C |
| ATOM | 9993 | OE1 | GLN | E | 131 | 14.858 | 28.094 | 26.318 | 1.00 | 91.57 | O |
| ATOM | 9994 | NE2 | GLN | E | 131 | 13.047 | 28.501 | 27.601 | 1.00 | 67.50 | N |
| ATOM | 9997 | C | GLN | E | 131 | 17.600 | 31.354 | 28.331 | 1.00 | 63.75 | C |
| ATOM | 9998 | O | GLN | E | 131 | 17.356 | 32.557 | 28.333 | 1.00 | 53.12 | O |
| ATOM | 9999 | N | GLU | E | 132 | 18.448 | 30.762 | 27.505 | 1.00 | 56.09 | N |
| ATOM | 10001 | CA | GLU | E | 132 | 19.043 | 31.485 | 26.410 | 1.00 | 65.03 | C |
| ATOM | 10003 | CB | GLU | E | 132 | 20.558 | 31.305 | 26.479 | 1.00 | 68.07 | C |
| ATOM | 10006 | CG | GLU | E | 132 | 21.272 | 32.458 | 27.162 | 1.00 | 86.15 | C |
| ATOM | 10009 | CD | GLU | E | 132 | 22.208 | 33.189 | 26.211 | 1.00 | 106.56 | C |
| ATOM | 10010 | OE1 | GLU | E | 132 | 22.869 | 34.149 | 26.670 | 1.00 | 114.80 | O |
| ATOM | 10011 | OE2 | GLU | E | 132 | 22.293 | 32.807 | 25.013 | 1.00 | 92.03 | O |
| ATOM | 10012 | C | GLU | E | 132 | 18.456 | 30.952 | 25.101 | 1.00 | 61.01 | C |
| ATOM | 10013 | O | GLU | E | 132 | 18.379 | 29.749 | 24.894 | 1.00 | 62.93 | O |
| ATOM | 10014 | N | TYR | E | 133 | 18.012 | 31.843 | 24.225 | 1.00 | 57.77 | N |
| ATOM | 10016 | CA | TYR | E | 133 | 17.284 | 31.426 | 23.033 | 1.00 | 53.58 | C |
| ATOM | 10018 | CB | TYR | E | 133 | 16.141 | 32.414 | 22.761 | 1.00 | 54.34 | C |
| ATOM | 10021 | CG | TYR | E | 133 | 15.065 | 32.356 | 23.827 | 1.00 | 45.53 | C |
| ATOM | 10022 | CD1 | TYR | E | 133 | 15.121 | 33.149 | 24.944 | 1.00 | 27.18 | C |
| ATOM | 10024 | CE1 | TYR | E | 133 | 14.169 | 33.085 | 25.912 | 1.00 | 29.12 | C |
| ATOM | 10026 | CZ | TYR | E | 133 | 13.130 | 32.197 | 25.806 | 1.00 | 32.12 | C |
| ATOM | 10027 | OH | TYR | E | 133 | 12.178 | 32.071 | 26.805 | 1.00 | 46.73 | O |
| ATOM | 10029 | CE2 | TYR | E | 133 | 13.033 | 31.403 | 24.700 | 1.00 | 43.81 | C |
| ATOM | 10031 | CD2 | TYR | E | 133 | 14.023 | 31.464 | 23.732 | 1.00 | 55.28 | C |
| ATOM | 10033 | C | TYR | E | 133 | 18.205 | 31.282 | 21.823 | 1.00 | 45.07 | C |
| ATOM | 10034 | O | TYR | E | 133 | 19.152 | 32.039 | 21.639 | 1.00 | 47.38 | O |
| ATOM | 10035 | N | SER | E | 134 | 17.912 | 30.340 | 20.941 | 1.00 | 43.06 | N |
| ATOM | 10037 | CA | SER | E | 134 | 18.678 | 30.278 | 19.693 | 1.00 | 42.98 | C |
| ATOM | 10039 | CB | SER | E | 134 | 18.180 | 29.174 | 18.772 | 1.00 | 38.65 | C |
| ATOM | 10042 | OG | SER | E | 134 | 18.239 | 27.949 | 19.483 | 1.00 | 35.45 | O |
| ATOM | 10044 | C | SER | E | 134 | 18.786 | 31.592 | 18.942 | 1.00 | 45.68 | C |
| ATOM | 10045 | O | SER | E | 134 | 19.881 | 31.962 | 18.527 | 1.00 | 50.26 | O |
| ATOM | 10046 | N | CYS | E | 135 | 17.663 | 32.289 | 18.756 | 1.00 | 51.98 | N |
| ATOM | 10048 | CA | CYS | E | 135 | 17.564 | 33.386 | 17.780 | 1.00 | 49.08 | C |
| ATOM | 10050 | CB | CYS | E | 135 | 16.723 | 32.945 | 16.594 | 1.00 | 44.69 | C |
| ATOM | 10053 | SG | CYS | E | 135 | 17.395 | 33.252 | 14.963 | 1.00 | 84.25 | S |
| ATOM | 10054 | C | CYS | E | 135 | 16.819 | 34.465 | 18.505 | 1.00 | 37.53 | C |
| ATOM | 10055 | O | CYS | E | 135 | 15.748 | 34.174 | 19.036 | 1.00 | 31.24 | O |
| ATOM | 10056 | N | VAL | E | 136 | 17.367 | 35.679 | 18.557 | 1.00 | 36.70 | N |
| ATOM | 10058 | CA | VAL | E | 136 | 16.628 | 36.793 | 19.188 | 1.00 | 39.94 | C |
| ATOM | 10060 | CB | VAL | E | 136 | 17.247 | 37.351 | 20.504 | 1.00 | 33.71 | C |
| ATOM | 10062 | CG1 | VAL | E | 136 | 16.587 | 38.649 | 20.889 | 1.00 | 37.89 | C |
| ATOM | 10066 | CG2 | VAL | E | 136 | 17.049 | 36.411 | 21.641 | 1.00 | 32.60 | C |
| ATOM | 10070 | C | VAL | E | 136 | 16.478 | 37.960 | 18.218 | 1.00 | 38.67 | C |
| ATOM | 10071 | O | VAL | E | 136 | 17.461 | 38.549 | 17.794 | 1.00 | 42.19 | O |
| ATOM | 10072 | N | VAL | E | 137 | 15.233 | 38.310 | 17.912 | 1.00 | 35.05 | N |
| ATOM | 10074 | CA | VAL | E | 137 | 14.958 | 39.288 | 16.886 | 1.00 | 38.04 | C |
| ATOM | 10076 | CB | VAL | E | 137 | 14.056 | 38.686 | 15.859 | 1.00 | 33.25 | C |
| ATOM | 10078 | CG1 | VAL | E | 137 | 13.724 | 39.717 | 14.827 | 1.00 | 39.22 | C |
| ATOM | 10082 | CG2 | VAL | E | 137 | 14.767 | 37.528 | 15.253 | 1.00 | 31.72 | C |
| ATOM | 10086 | C | VAL | E | 137 | 14.349 | 40.549 | 17.474 | 1.00 | 40.52 | C |
| ATOM | 10087 | O | VAL | E | 137 | 13.312 | 40.501 | 18.094 | 1.00 | 41.89 | O |
| ATOM | 10088 | N | LYS | E | 138 | 15.091 | 41.643 | 17.394 | 1.00 | 46.01 | N |
| ATOM | 10090 | CA | LYS | E | 138 | 14.614 | 42.958 | 17.747 | 1.00 | 43.70 | C |
| ATOM | 10092 | CB | LYS | E | 138 | 15.752 | 43.701 | 18.449 | 1.00 | 48.85 | C |
| ATOM | 10095 | CG | LYS | E | 138 | 15.370 | 45.003 | 19.172 | 1.00 | 63.35 | C |
| ATOM | 10098 | CD | LYS | E | 138 | 16.487 | 45.531 | 20.096 | 1.00 | 81.81 | C |
| ATOM | 10101 | CE | LYS | E | 138 | 16.764 | 47.048 | 19.938 | 1.00 | 95.77 | C |
| ATOM | 10104 | NZ | LYS | E | 138 | 16.962 | 47.779 | 21.247 | 1.00 | 100.76 | N |
| ATOM | 10108 | C | LYS | E | 138 | 14.245 | 43.640 | 16.430 | 1.00 | 42.59 | C |
| ATOM | 10109 | O | LYS | E | 138 | 15.035 | 43.646 | 15.488 | 1.00 | 49.55 | O |
| ATOM | 10110 | N | MET | E | 139 | 13.040 | 44.203 | 16.374 | 1.00 | 39.33 | N |
| ATOM | 10112 | CA | MET | E | 139 | 12.459 | 44.791 | 15.176 | 1.00 | 35.07 | C |
| ATOM | 10114 | CB | MET | E | 139 | 11.882 | 43.718 | 14.265 | 1.00 | 42.49 | C |
| ATOM | 10117 | CG | MET | E | 139 | 10.602 | 43.076 | 14.802 | 1.00 | 42.10 | C |
| ATOM | 10120 | SD | MET | E | 139 | 9.814 | 42.183 | 13.462 | 1.00 | 42.90 | S |
| ATOM | 10121 | CE | MET | E | 139 | 10.492 | 40.675 | 13.660 | 1.00 | 59.23 | C |
| ATOM | 10125 | C | MET | E | 139 | 11.322 | 45.689 | 15.625 | 1.00 | 45.13 | C |
| ATOM | 10126 | O | MET | E | 139 | 10.875 | 45.635 | 16.785 | 1.00 | 47.28 | O |
| ATOM | 10127 | N | PRO | E | 140 | 10.923 | 46.590 | 14.736 | 1.00 | 45.07 | N |
| ATOM | 10128 | CA | PRO | E | 140 | 9.970 | 47.641 | 15.082 | 1.00 | 42.55 | C |
| ATOM | 10130 | CB | PRO | E | 140 | 9.928 | 48.515 | 13.830 | 1.00 | 42.37 | C |
| ATOM | 10133 | CG | PRO | E | 140 | 11.149 | 48.214 | 13.154 | 1.00 | 36.55 | C |
| ATOM | 10136 | CD | PRO | E | 140 | 11.417 | 46.754 | 13.364 | 1.00 | 47.82 | C |
| ATOM | 10139 | C | PRO | E | 140 | 8.636 | 46.996 | 15.279 | 1.00 | 44.64 | C |
| ATOM | 10140 | O | PRO | E | 140 | 8.332 | 46.042 | 14.560 | 1.00 | 46.35 | O |
| ATOM | 10141 | N | SER | E | 141 | 7.888 | 47.489 | 16.254 | 1.00 | 44.54 | N |
| ATOM | 10143 | CA | SER | E | 141 | 6.733 | 46.766 | 16.753 | 1.00 | 39.50 | C |
| ATOM | 10145 | CB | SER | E | 141 | 6.341 | 47.323 | 18.119 | 1.00 | 40.01 | C |
| ATOM | 10148 | OG | SER | E | 141 | 6.089 | 48.714 | 18.114 | 1.00 | 25.86 | O |
| ATOM | 10150 | C | SER | E | 141 | 5.567 | 46.878 | 15.778 | 1.00 | 38.43 | C |
| ATOM | 10151 | O | SER | E | 141 | 4.851 | 45.918 | 15.541 | 1.00 | 49.94 | O |
| ATOM | 10152 | N | GLY | E | 142 | 5.364 | 48.063 | 15.232 | 1.00 | 36.51 | N |
| ATOM | 10154 | CA | GLY | E | 142 | 4.429 | 48.251 | 14.148 | 1.00 | 36.16 | C |
| ATOM | 10157 | C | GLY | E | 142 | 4.657 | 47.355 | 12.943 | 1.00 | 42.12 | C |
| ATOM | 10158 | O | GLY | E | 142 | 3.698 | 46.765 | 12.429 | 1.00 | 46.00 | O |
| ATOM | 10159 | N | GLU | E | 143 | 5.900 | 47.199 | 12.490 | 1.00 | 35.45 | N |
| ATOM | 10161 | CA | GLU | E | 143 | 6.165 | 46.196 | 11.467 | 1.00 | 27.65 | C |
| ATOM | 10163 | CB | GLU | E | 143 | 7.666 | 46.106 | 11.197 | 1.00 | 34.14 | C |
| ATOM | 10166 | CG | GLU | E | 143 | 8.035 | 45.316 | 9.947 | 1.00 | 46.37 | C |
| ATOM | 10169 | CD | GLU | E | 143 | 7.431 | 45.904 | 8.687 | 1.00 | 51.82 | C |
| ATOM | 10170 | OE1 | GLU | E | 143 | 7.748 | 47.061 | 8.344 | 1.00 | 74.77 | O |
| ATOM | 10171 | OE2 | GLU | E | 143 | 6.598 | 45.221 | 8.069 | 1.00 | 52.72 | O |
| ATOM | 10172 | C | GLU | E | 143 | 5.650 | 44.810 | 11.862 | 1.00 | 35.03 | C |
| ATOM | 10173 | O | GLU | E | 143 | 5.000 | 44.119 | 11.054 | 1.00 | 33.69 | O |
| ATOM | 10174 | N | PHE | E | 144 | 6.027 | 44.353 | 13.061 | 1.00 | 35.45 | N |
| ATOM | 10176 | CA | PHE | E | 144 | 5.672 | 43.002 | 13.477 | 1.00 | 36.21 | C |
| ATOM | 10178 | CB | PHE | E | 144 | 6.274 | 42.709 | 14.843 | 1.00 | 36.50 | C |
| ATOM | 10181 | CG | PHE | E | 144 | 5.915 | 41.364 | 15.385 | 1.00 | 29.12 | C |
| ATOM | 10182 | CD1 | PHE | E | 144 | 6.161 | 40.225 | 14.658 | 1.00 | 23.31 | C |
| ATOM | 10184 | CE1 | PHE | E | 144 | 5.801 | 38.994 | 15.144 | 1.00 | 14.38 | C |
| ATOM | 10186 | CZ | PHE | E | 144 | 5.118 | 38.904 | 16.326 | 1.00 | 31.06 | C |
| ATOM | 10188 | CE2 | PHE | E | 144 | 4.881 | 40.022 | 17.070 | 1.00 | 26.50 | C |
| ATOM | 10190 | CD2 | PHE | E | 144 | 5.243 | 41.255 | 16.579 | 1.00 | 30.76 | C |
| ATOM | 10192 | C | PHE | E | 144 | 4.138 | 42.890 | 13.514 | 1.00 | 38.14 | C |
| ATOM | 10193 | O | PHE | E | 144 | 3.512 | 41.897 | 13.119 | 1.00 | 39.16 | O |
| ATOM | 10194 | N | ALA | E | 145 | 3.516 | 43.982 | 13.903 | 1.00 | 28.45 | N |
| ATOM | 10196 | CA | ALA | E | 145 | 2.089 | 43.997 | 13.866 | 1.00 | 29.03 | C |
| ATOM | 10198 | CB | ALA | E | 145 | 1.626 | 45.173 | 14.626 | 1.00 | 25.54 | C |
| ATOM | 10202 | C | ALA | E | 145 | 1.488 | 43.936 | 12.455 | 1.00 | 27.38 | C |
| ATOM | 10203 | O | ALA | E | 145 | 0.483 | 43.281 | 12.254 | 1.00 | 37.03 | O |
| ATOM | 10204 | N | ARG | E | 146 | 2.048 | 44.628 | 11.480 | 1.00 | 32.55 | N |
| ATOM | 10206 | CA | ARG | E | 146 | 1.515 | 44.576 | 10.123 | 1.00 | 32.99 | C |
| ATOM | 10208 | CB | ARG | E | 146 | 2.239 | 45.557 | 9.171 | 1.00 | 45.16 | C |
| ATOM | 10211 | CG | ARG | E | 146 | 1.895 | 47.066 | 9.341 | 1.00 | 68.37 | C |
| ATOM | 10214 | CD | ARG | E | 146 | 2.591 | 48.066 | 8.373 | 1.00 | 70.20 | C |
| ATOM | 10217 | NE | ARG | E | 146 | 2.103 | 47.975 | 6.990 | 1.00 | 85.55 | N |
| ATOM | 10219 | CZ | ARG | E | 146 | 1.048 | 48.619 | 6.465 | 1.00 | 95.37 | C |
| ATOM | 10220 | NH1 | ARG | E | 146 | 0.270 | 49.427 | 7.187 | 1.00 | 88.72 | N |
| ATOM | 10223 | NH2 | ARG | E | 146 | 0.763 | 48.443 | 5.177 | 1.00 | 102.95 | N |
| ATOM | 10226 | C | ARG | E | 146 | 1.756 | 43.165 | 9.612 | 1.00 | 34.52 | C |
| ATOM | 10227 | O | ARG | E | 146 | 0.882 | 42.576 | 8.982 | 1.00 | 34.67 | O |
| ATOM | 10228 | N | ILE | E | 147 | 2.947 | 42.614 | 9.845 | 1.00 | 36.00 | N |
| ATOM | 10230 | CA | ILE | E | 147 | 3.215 | 41.285 | 9.317 | 1.00 | 33.03 | C |
| ATOM | 10232 | CB | ILE | E | 147 | 4.595 | 40.800 | 9.705 | 1.00 | 29.19 | C |
| ATOM | 10234 | CG1 | ILE | E | 147 | 5.609 | 41.215 | 8.650 | 1.00 | 34.27 | C |
| ATOM | 10237 | CD1 | ILE | E | 147 | 6.933 | 41.455 | 9.146 | 1.00 | 28.57 | C |
| ATOM | 10241 | CG2 | ILE | E | 147 | 4.614 | 39.300 | 9.592 | 1.00 | 29.71 | C |
| ATOM | 10245 | C | ILE | E | 147 | 2.194 | 40.240 | 9.740 | 1.00 | 33.95 | C |
| ATOM | 10246 | O | ILE | E | 147 | 1.729 | 39.472 | 8.927 | 1.00 | 38.88 | O |
| ATOM | 10247 | N | CYS | E | 148 | 1.874 | 40.206 | 11.026 | 1.00 | 37.47 | N |
| ATOM | 10249 | CA | CYS | E | 148 | 0.930 | 39.257 | 11.584 | 1.00 | 37.81 | C |
| ATOM | 10251 | CB | CYS | E | 148 | 0.986 | 39.338 | 13.117 | 1.00 | 41.48 | C |
| ATOM | 10254 | SG | CYS | E | 148 | 2.484 | 38.761 | 13.978 | 1.00 | 35.47 | S |
| ATOM | 10255 | C | CYS | E | 148 | −0.510 | 39.544 | 11.121 | 1.00 | 34.71 | C |
| ATOM | 10256 | O | CYS | E | 148 | −1.357 | 38.651 | 10.989 | 1.00 | 27.76 | O |
| ATOM | 10257 | N | ARG | E | 149 | −0.851 | 40.803 | 10.941 | 1.00 | 31.54 | N |
| ATOM | 10259 | CA | ARG | E | 149 | −2.188 | 41.051 | 10.423 | 1.00 | 36.99 | C |
| ATOM | 10261 | CB | ARG | E | 149 | −2.637 | 42.475 | 10.742 | 1.00 | 29.21 | C |
| ATOM | 10264 | CG | ARG | E | 149 | −3.742 | 43.041 | 9.920 | 1.00 | 49.04 | C |
| ATOM | 10267 | CD | ARG | E | 149 | −3.777 | 44.570 | 9.967 | 1.00 | 73.13 | C |
| ATOM | 10270 | NE | ARG | E | 149 | −3.251 | 45.169 | 8.732 | 1.00 | 89.86 | N |
| ATOM | 10272 | CZ | ARG | E | 149 | −2.229 | 46.025 | 8.665 | 1.00 | 90.78 | C |
| ATOM | 10273 | NH1 | ARG | E | 149 | −1.575 | 46.390 | 9.769 | 1.00 | 91.40 | N |
| ATOM | 10276 | NH2 | ARG | E | 149 | −1.847 | 46.501 | 7.483 | 1.00 | 75.54 | N |
| ATOM | 10279 | C | ARG | E | 149 | −2.265 | 40.641 | 8.941 | 1.00 | 37.46 | C |
| ATOM | 10280 | O | ARG | E | 149 | −3.149 | 39.872 | 8.535 | 1.00 | 37.36 | O |
| ATOM | 10281 | N | ASP | E | 150 | −1.283 | 41.043 | 8.143 | 1.00 | 40.15 | N |
| ATOM | 10283 | CA | ASP | E | 150 | −1.252 | 40.629 | 6.740 | 1.00 | 37.57 | C |
| ATOM | 10285 | CB | ASP | E | 150 | −0.059 | 41.272 | 6.070 | 1.00 | 36.12 | C |
| ATOM | 10288 | CG | ASP | E | 150 | −0.172 | 42.789 | 5.996 | 1.00 | 42.04 | C |
| ATOM | 10289 | OD1 | ASP | E | 150 | −1.237 | 43.360 | 6.305 | 1.00 | 58.89 | O |
| ATOM | 10290 | OD2 | ASP | E | 150 | 0.786 | 43.518 | 5.676 | 1.00 | 43.33 | O |
| ATOM | 10291 | C | ASP | E | 150 | −1.251 | 39.100 | 6.528 | 1.00 | 40.87 | C |
| ATOM | 10292 | O | ASP | E | 150 | −1.968 | 38.591 | 5.659 | 1.00 | 39.56 | O |
| ATOM | 10293 | N | LEU | E | 151 | −0.490 | 38.346 | 7.315 | 1.00 | 29.67 | N |
| ATOM | 10295 | CA | LEU | E | 151 | −0.328 | 36.946 | 6.974 | 1.00 | 33.30 | C |
| ATOM | 10297 | CB | LEU | E | 151 | 0.945 | 36.303 | 7.521 | 1.00 | 31.85 | C |
| ATOM | 10300 | CG | LEU | E | 151 | 2.241 | 36.788 | 6.845 | 1.00 | 35.02 | C |
| ATOM | 10302 | CD1 | LEU | E | 151 | 3.494 | 36.232 | 7.415 | 1.00 | 41.42 | C |
| ATOM | 10306 | CD2 | LEU | E | 151 | 2.287 | 36.532 | 5.387 | 1.00 | 41.84 | C |
| ATOM | 10310 | C | LEU | E | 151 | −1.528 | 36.246 | 7.501 | 1.00 | 37.66 | C |
| ATOM | 10311 | O | LEU | E | 151 | −1.926 | 35.219 | 6.961 | 1.00 | 44.66 | O |
| ATOM | 10312 | N | SER | E | 152 | −2.158 | 36.849 | 8.499 | 1.00 | 46.70 | N |
| ATOM | 10314 | CA | SER | E | 152 | −3.313 | 36.208 | 9.127 | 1.00 | 46.00 | C |
| ATOM | 10316 | CB | SER | E | 152 | −3.809 | 36.974 | 10.345 | 1.00 | 33.30 | C |
| ATOM | 10319 | OG | SER | E | 152 | −4.447 | 38.124 | 9.860 | 1.00 | 35.85 | O |
| ATOM | 10321 | C | SER | E | 152 | −4.440 | 36.099 | 8.121 | 1.00 | 41.45 | C |
| ATOM | 10322 | O | SER | E | 152 | −5.328 | 35.288 | 8.316 | 1.00 | 54.40 | O |
| ATOM | 10323 | N | HIS | E | 153 | −4.394 | 36.903 | 7.065 | 1.00 | 45.23 | N |
| ATOM | 10325 | CA | HIS | E | 153 | −5.421 | 36.929 | 6.009 | 1.00 | 47.78 | C |
| ATOM | 10327 | CB | HIS | E | 153 | −5.542 | 38.347 | 5.424 | 1.00 | 53.32 | C |
| ATOM | 10330 | CG | HIS | E | 153 | −6.152 | 39.345 | 6.365 | 1.00 | 60.37 | C |
| ATOM | 10331 | ND1 | HIS | E | 153 | −7.324 | 39.107 | 7.052 | 1.00 | 71.29 | N |
| ATOM | 10333 | CE1 | HIS | E | 153 | −7.612 | 40.147 | 7.813 | 1.00 | 75.53 | C |
| ATOM | 10335 | NE2 | HIS | E | 153 | −6.675 | 41.061 | 7.632 | 1.00 | 81.82 | N |
| ATOM | 10337 | CD2 | HIS | E | 153 | −5.751 | 40.584 | 6.732 | 1.00 | 72.08 | C |
| ATOM | 10339 | C | HIS | E | 153 | −5.105 | 36.000 | 4.853 | 1.00 | 40.43 | C |
| ATOM | 10340 | O | HIS | E | 153 | −5.905 | 35.778 | 3.973 | 1.00 | 49.66 | O |
| ATOM | 10341 | N | ILE | E | 154 | −3.896 | 35.478 | 4.851 | 1.00 | 41.75 | N |
| ATOM | 10343 | CA | ILE | E | 154 | −3.530 | 34.408 | 3.958 | 1.00 | 35.23 | C |
| ATOM | 10345 | CB | ILE | E | 154 | −2.073 | 34.624 | 3.514 | 1.00 | 37.30 | C |
| ATOM | 10347 | CG1 | ILE | E | 154 | −1.976 | 35.613 | 2.351 | 1.00 | 34.61 | C |
| ATOM | 10350 | CD1 | ILE | E | 154 | −1.438 | 36.962 | 2.744 | 1.00 | 47.16 | C |
| ATOM | 10354 | CG2 | ILE | E | 154 | −1.468 | 33.328 | 3.050 | 1.00 | 48.46 | C |
| ATOM | 10358 | C | ILE | E | 154 | −3.672 | 33.059 | 4.664 | 1.00 | 38.68 | C |
| ATOM | 10359 | O | ILE | E | 154 | −4.026 | 32.049 | 4.063 | 1.00 | 41.84 | O |
| ATOM | 10360 | N | GLY | E | 155 | −3.371 | 32.986 | 5.947 | 1.00 | 39.60 | N |
| ATOM | 10362 | CA | GLY | E | 155 | −3.313 | 31.668 | 6.556 | 1.00 | 40.43 | C |
| ATOM | 10365 | C | GLY | E | 155 | −3.461 | 31.783 | 8.046 | 1.00 | 37.98 | C |
| ATOM | 10366 | O | GLY | E | 155 | −3.610 | 32.886 | 8.555 | 1.00 | 39.98 | O |
| ATOM | 10367 | N | ASP | E | 156 | −3.483 | 30.662 | 8.750 | 1.00 | 42.99 | N |
| ATOM | 10369 | CA | ASP | E | 156 | −3.837 | 30.728 | 10.158 | 1.00 | 38.09 | C |
| ATOM | 10371 | CB | ASP | E | 156 | −5.252 | 30.186 | 10.462 | 1.00 | 45.63 | C |
| ATOM | 10374 | CG | ASP | E | 156 | −5.376 | 28.681 | 10.405 | 1.00 | 57.57 | C |
| ATOM | 10375 | OD1 | ASP | E | 156 | −6.322 | 28.223 | 9.735 | 1.00 | 84.25 | O |
| ATOM | 10376 | OD2 | ASP | E | 156 | −4.705 | 27.884 | 11.092 | 1.00 | 86.26 | O |
| ATOM | 10377 | C | ASP | E | 156 | −2.694 | 30.341 | 11.067 | 1.00 | 30.39 | C |
| ATOM | 10378 | O | ASP | E | 156 | −2.604 | 30.800 | 12.194 | 1.00 | 44.24 | O |
| ATOM | 10379 | N | ALA | E | 157 | −1.656 | 29.806 | 10.444 | 1.00 | 36.60 | N |
| ATOM | 10381 | CA | ALA | E | 157 | −0.304 | 29.823 | 10.985 | 1.00 | 32.99 | C |
| ATOM | 10383 | CB | ALA | E | 157 | 0.186 | 28.409 | 11.022 | 1.00 | 35.78 | C |
| ATOM | 10387 | C | ALA | E | 157 | 0.754 | 30.640 | 10.240 | 1.00 | 38.08 | C |
| ATOM | 10388 | O | ALA | E | 157 | 0.711 | 30.782 | 9.007 | 1.00 | 44.60 | O |
| ATOM | 10389 | N | VAL | E | 158 | 1.814 | 30.968 | 10.973 | 1.00 | 28.85 | N |
| ATOM | 10391 | CA | VAL | E | 158 | 2.968 | 31.648 | 10.392 | 1.00 | 33.09 | C |
| ATOM | 10393 | CB | VAL | E | 158 | 3.266 | 33.134 | 10.938 | 1.00 | 40.16 | C |
| ATOM | 10395 | CG1 | VAL | E | 158 | 3.519 | 33.247 | 12.448 | 1.00 | 35.70 | C |
| ATOM | 10399 | CG2 | VAL | E | 158 | 4.491 | 33.695 | 10.297 | 1.00 | 33.62 | C |
| ATOM | 10403 | C | VAL | E | 158 | 4.155 | 30.812 | 10.721 | 1.00 | 31.74 | C |
| ATOM | 10404 | O | VAL | E | 158 | 4.232 | 30.280 | 11.824 | 1.00 | 37.46 | O |
| ATOM | 10405 | N | VAL | E | 159 | 5.080 | 30.739 | 9.772 | 1.00 | 34.93 | N |
| ATOM | 10407 | CA | VAL | E | 159 | 6.320 | 30.036 | 9.984 | 1.00 | 36.67 | C |
| ATOM | 10409 | CB | VAL | E | 159 | 6.616 | 29.002 | 8.893 | 1.00 | 37.62 | C |
| ATOM | 10411 | CG1 | VAL | E | 159 | 7.831 | 28.184 | 9.287 | 1.00 | 32.03 | C |
| ATOM | 10415 | CG2 | VAL | E | 159 | 5.450 | 28.043 | 8.762 | 1.00 | 35.84 | C |
| ATOM | 10419 | C | VAL | E | 159 | 7.435 | 31.044 | 10.090 | 1.00 | 39.77 | C |
| ATOM | 10420 | O | VAL | E | 159 | 7.717 | 31.731 | 9.133 | 1.00 | 39.95 | O |
| ATOM | 10421 | N | ILE | E | 160 | 8.029 | 31.156 | 11.278 | 1.00 | 43.94 | N |
| ATOM | 10423 | CA | ILE | E | 160 | 9.125 | 32.083 | 11.480 | 1.00 | 45.11 | C |
| ATOM | 10425 | CB | ILE | E | 160 | 9.032 | 32.711 | 12.836 | 1.00 | 38.28 | C |
| ATOM | 10427 | CG1 | ILE | E | 160 | 7.645 | 33.329 | 12.992 | 1.00 | 34.42 | C |
| ATOM | 10430 | CD1 | ILE | E | 160 | 7.417 | 33.941 | 14.346 | 1.00 | 41.65 | C |
| ATOM | 10434 | CG2 | ILE | E | 160 | 10.086 | 33.793 | 12.942 | 1.00 | 41.17 | C |
| ATOM | 10438 | C | ILE | E | 160 | 10.470 | 31.390 | 11.304 | 1.00 | 52.97 | C |
| ATOM | 10439 | O | ILE | E | 160 | 10.707 | 30.353 | 11.915 | 1.00 | 51.90 | O |
| ATOM | 10440 | N | SER | E | 161 | 11.274 | 31.911 | 10.376 | 1.00 | 55.09 | N |
| ATOM | 10442 | CA | SER | E | 161 | 12.533 | 31.298 | 9.939 | 1.00 | 50.98 | C |
| ATOM | 10444 | CB | SER | E | 161 | 12.574 | 30.987 | 8.436 | 1.00 | 45.60 | C |
| ATOM | 10447 | OG | SER | E | 161 | 12.099 | 29.695 | 8.128 | 1.00 | 51.61 | O |
| ATOM | 10449 | C | SER | E | 161 | 13.555 | 32.387 | 10.145 | 1.00 | 52.37 | C |
| ATOM | 10450 | O | SER | E | 161 | 13.430 | 33.483 | 9.589 | 1.00 | 52.60 | O |
| ATOM | 10451 | N | CYS | E | 162 | 14.572 | 32.089 | 10.942 | 1.00 | 56.67 | N |
| ATOM | 10453 | CA | CYS | E | 162 | 15.465 | 33.139 | 11.394 | 1.00 | 53.82 | C |
| ATOM | 10455 | CB | CYS | E | 162 | 15.119 | 33.449 | 12.836 | 1.00 | 46.71 | C |
| ATOM | 10458 | SG | CYS | E | 162 | 16.287 | 34.500 | 13.642 | 1.00 | 68.44 | S |
| ATOM | 10459 | C | CYS | E | 162 | 16.864 | 32.608 | 11.195 | 1.00 | 50.61 | C |
| ATOM | 10460 | O | CYS | E | 162 | 17.106 | 31.450 | 11.503 | 1.00 | 47.01 | O |
| ATOM | 10461 | N | ALA | E | 163 | 17.685 | 33.394 | 10.503 | 1.00 | 57.48 | N |
| ATOM | 10463 | CA | ALA | E | 163 | 19.113 | 33.145 | 10.305 | 1.00 | 62.94 | C |
| ATOM | 10465 | CB | ALA | E | 163 | 19.368 | 32.418 | 8.963 | 1.00 | 62.58 | C |
| ATOM | 10469 | C | ALA | E | 163 | 19.827 | 34.506 | 10.333 | 1.00 | 68.38 | C |
| ATOM | 10470 | O | ALA | E | 163 | 19.186 | 35.545 | 10.503 | 1.00 | 68.75 | O |
| ATOM | 10471 | N | LYS | E | 164 | 21.151 | 34.505 | 10.195 | 1.00 | 70.60 | N |
| ATOM | 10473 | CA | LYS | E | 164 | 21.947 | 35.677 | 10.543 | 1.00 | 72.38 | C |
| ATOM | 10475 | CB | LYS | E | 164 | 23.436 | 35.293 | 10.535 | 1.00 | 76.47 | C |
| ATOM | 10478 | CG | LYS | E | 164 | 24.432 | 36.471 | 10.559 | 1.00 | 98.55 | C |
| ATOM | 10481 | CD | LYS | E | 164 | 24.783 | 36.956 | 11.994 | 1.00 | 113.83 | C |
| ATOM | 10484 | CE | LYS | E | 164 | 25.389 | 38.380 | 12.019 | 1.00 | 108.52 | C |
| ATOM | 10487 | NZ | LYS | E | 164 | 26.577 | 38.535 | 12.927 | 1.00 | 94.44 | N |
| ATOM | 10491 | C | LYS | E | 164 | 21.651 | 36.788 | 9.522 | 1.00 | 73.97 | C |
| ATOM | 10492 | O | LYS | E | 164 | 21.615 | 37.977 | 9.840 | 1.00 | 60.21 | O |
| ATOM | 10493 | N | ASP | E | 165 | 21.466 | 36.375 | 8.274 | 1.00 | 77.45 | N |
| ATOM | 10495 | CA | ASP | E | 165 | 20.975 | 37.249 | 7.221 | 1.00 | 81.85 | C |
| ATOM | 10497 | CB | ASP | E | 165 | 20.497 | 36.354 | 6.055 | 1.00 | 91.74 | C |
| ATOM | 10500 | CG | ASP | E | 165 | 19.979 | 37.153 | 4.840 | 1.00 | 114.51 | C |
| ATOM | 10501 | OD1 | ASP | E | 165 | 20.741 | 37.326 | 3.857 | 1.00 | 137.83 | O |
| ATOM | 10502 | OD2 | ASP | E | 165 | 18.815 | 37.616 | 4.748 | 1.00 | 132.86 | O |
| ATOM | 10503 | C | ASP | E | 165 | 19.839 | 38.175 | 7.709 | 1.00 | 71.55 | C |
| ATOM | 10504 | O | ASP | E | 165 | 19.888 | 39.380 | 7.531 | 1.00 | 67.90 | O |
| ATOM | 10505 | N | GLY | E | 166 | 18.772 | 37.608 | 8.257 | 1.00 | 64.68 | N |
| ATOM | 10507 | CA | GLY | E | 166 | 17.434 | 38.136 | 8.041 | 1.00 | 57.28 | C |
| ATOM | 10510 | C | GLY | E | 166 | 16.415 | 37.235 | 8.716 | 1.00 | 53.15 | C |
| ATOM | 10511 | O | GLY | E | 166 | 16.729 | 36.077 | 8.997 | 1.00 | 54.27 | O |
| ATOM | 10512 | N | VAL | E | 167 | 15.218 | 37.749 | 8.996 | 1.00 | 45.32 | N |
| ATOM | 10514 | CA | VAL | E | 167 | 14.131 | 36.894 | 9.444 | 1.00 | 39.59 | C |
| ATOM | 10516 | CB | VAL | E | 167 | 13.570 | 37.314 | 10.798 | 1.00 | 35.18 | C |
| ATOM | 10518 | CG1 | VAL | E | 167 | 12.853 | 38.643 | 10.722 | 1.00 | 52.40 | C |
| ATOM | 10522 | CG2 | VAL | E | 167 | 12.635 | 36.256 | 11.337 | 1.00 | 41.27 | C |
| ATOM | 10526 | C | VAL | E | 167 | 13.048 | 36.785 | 8.371 | 1.00 | 45.46 | C |
| ATOM | 10527 | O | VAL | E | 167 | 12.824 | 37.729 | 7.635 | 1.00 | 43.91 | O |
| ATOM | 10528 | N | LYS | E | 168 | 12.447 | 35.598 | 8.249 | 1.00 | 46.79 | N |
| ATOM | 10530 | CA | LYS | E | 168 | 11.374 | 35.334 | 7.308 | 1.00 | 44.71 | C |
| ATOM | 10532 | CB | LYS | E | 168 | 11.842 | 34.273 | 6.313 | 1.00 | 49.05 | C |
| ATOM | 10535 | CG | LYS | E | 168 | 10.778 | 33.763 | 5.313 | 1.00 | 46.67 | C |
| ATOM | 10538 | CD | LYS | E | 168 | 11.379 | 33.413 | 3.950 | 1.00 | 46.75 | C |
| ATOM | 10541 | CE | LYS | E | 168 | 11.846 | 31.967 | 3.853 | 1.00 | 62.73 | C |
| ATOM | 10544 | NZ | LYS | E | 168 | 10.926 | 31.189 | 2.976 | 1.00 | 76.28 | N |
| ATOM | 10548 | C | LYS | E | 168 | 10.094 | 34.849 | 8.000 | 1.00 | 44.98 | C |
| ATOM | 10549 | O | LYS | E | 168 | 10.139 | 34.042 | 8.928 | 1.00 | 47.21 | O |
| ATOM | 10550 | N | PHE | E | 169 | 8.961 | 35.341 | 7.507 | 1.00 | 38.41 | N |
| ATOM | 10552 | CA | PHE | E | 169 | 7.642 | 35.024 | 8.032 | 1.00 | 36.24 | C |
| ATOM | 10554 | CB | PHE | E | 169 | 6.950 | 36.278 | 8.563 | 1.00 | 30.09 | C |
| ATOM | 10557 | CG | PHE | E | 169 | 7.675 | 36.931 | 9.698 | 1.00 | 34.35 | C |
| ATOM | 10558 | CD1 | PHE | E | 169 | 8.732 | 37.791 | 9.470 | 1.00 | 33.86 | C |
| ATOM | 10560 | CE1 | PHE | E | 169 | 9.396 | 38.383 | 10.526 | 1.00 | 36.65 | C |
| ATOM | 10562 | CZ | PHE | E | 169 | 9.029 | 38.093 | 11.823 | 1.00 | 47.11 | C |
| ATOM | 10564 | CE2 | PHE | E | 169 | 7.986 | 37.234 | 12.062 | 1.00 | 47.56 | C |
| ATOM | 10566 | CD2 | PHE | E | 169 | 7.298 | 36.679 | 11.002 | 1.00 | 41.95 | C |
| ATOM | 10568 | C | PHE | E | 169 | 6.805 | 34.554 | 6.866 | 1.00 | 37.30 | C |
| ATOM | 10569 | O | PHE | E | 169 | 6.625 | 35.305 | 5.901 | 1.00 | 37.99 | O |
| ATOM | 10570 | N | SER | E | 170 | 6.212 | 33.371 | 7.023 | 1.00 | 36.12 | N |
| ATOM | 10572 | CA | SER | E | 170 | 5.522 | 32.709 | 5.944 | 1.00 | 35.68 | C |
| ATOM | 10574 | CB | SER | E | 170 | 6.390 | 31.608 | 5.365 | 1.00 | 30.58 | C |
| ATOM | 10577 | OG | SER | E | 170 | 7.656 | 32.078 | 4.972 | 1.00 | 49.32 | O |
| ATOM | 10579 | C | SER | E | 170 | 4.199 | 32.131 | 6.443 | 1.00 | 39.55 | C |
| ATOM | 10580 | O | SER | E | 170 | 4.128 | 31.652 | 7.555 | 1.00 | 43.53 | O |
| ATOM | 10581 | N | ALA | E | 171 | 3.162 | 32.230 | 5.616 | 1.00 | 36.52 | N |
| ATOM | 10583 | CA | ALA | E | 171 | 1.949 | 31.485 | 5.803 | 1.00 | 39.20 | C |
| ATOM | 10585 | CB | ALA | E | 171 | 0.956 | 32.382 | 6.468 | 1.00 | 48.06 | C |
| ATOM | 10589 | C | ALA | E | 171 | 1.340 | 30.981 | 4.492 | 1.00 | 41.45 | C |
| ATOM | 10590 | O | ALA | E | 171 | 1.642 | 31.518 | 3.435 | 1.00 | 36.60 | O |
| ATOM | 10591 | N | SER | E | 172 | 0.397 | 30.038 | 4.595 | 1.00 | 36.39 | N |
| ATOM | 10593 | CA | SER | E | 172 | −0.359 | 29.563 | 3.450 | 1.00 | 41.97 | C |
| ATOM | 10595 | CB | SER | E | 172 | 0.379 | 28.406 | 2.837 | 1.00 | 35.75 | C |
| ATOM | 10598 | OG | SER | E | 172 | 0.547 | 27.498 | 3.882 | 1.00 | 62.18 | O |
| ATOM | 10600 | C | SER | E | 172 | −1.755 | 29.073 | 3.809 | 1.00 | 41.64 | C |
| ATOM | 10601 | O | SER | E | 172 | −2.052 | 28.875 | 4.966 | 1.00 | 54.83 | O |
| ATOM | 10602 | N | GLY | E | 173 | −2.611 | 28.935 | 2.804 | 1.00 | 49.85 | N |
| ATOM | 10604 | CA | GLY | E | 173 | −4.047 | 28.846 | 2.984 | 1.00 | 54.94 | C |
| ATOM | 10607 | C | GLY | E | 173 | −4.822 | 28.352 | 1.759 | 1.00 | 63.05 | C |
| ATOM | 10608 | O | GLY | E | 173 | −4.300 | 27.684 | 0.874 | 1.00 | 61.51 | O |
| ATOM | 10609 | N | GLU | E | 174 | −6.108 | 28.680 | 1.700 | 1.00 | 69.20 | N |
| ATOM | 10611 | CA | GLU | E | 174 | −6.912 | 28.277 | 0.555 | 1.00 | 69.89 | C |
| ATOM | 10613 | CB | GLU | E | 174 | −8.405 | 28.230 | 0.934 | 1.00 | 70.72 | C |
| ATOM | 10616 | CG | GLU | E | 174 | −8.758 | 27.148 | 1.961 | 1.00 | 86.85 | C |
| ATOM | 10619 | CD | GLU | E | 174 | −9.229 | 25.812 | 1.372 | 1.00 | 96.95 | C |
| ATOM | 10620 | OE1 | GLU | E | 174 | −8.386 | 25.010 | 0.901 | 1.00 | 86.28 | O |
| ATOM | 10621 | OE2 | GLU | E | 174 | −10.446 | 25.511 | 1.429 | 1.00 | 101.96 | O |
| ATOM | 10622 | C | GLU | E | 174 | −6.646 | 29.124 | −0.699 | 1.00 | 63.46 | C |
| ATOM | 10623 | O | GLU | E | 174 | −6.522 | 28.573 | −1.778 | 1.00 | 62.30 | O |
| ATOM | 10624 | N | LEU | E | 175 | −6.510 | 30.441 | −0.549 | 1.00 | 64.00 | N |
| ATOM | 10626 | CA | LEU | E | 175 | −6.303 | 31.374 | −1.672 | 1.00 | 62.87 | C |
| ATOM | 10628 | CB | LEU | E | 175 | −6.711 | 32.790 | −1.259 | 1.00 | 63.61 | C |
| ATOM | 10631 | CG | LEU | E | 175 | −5.864 | 33.709 | −0.379 | 1.00 | 52.55 | C |
| ATOM | 10633 | CD1 | LEU | E | 175 | −4.823 | 34.381 | −1.202 | 1.00 | 44.08 | C |
| ATOM | 10637 | CD2 | LEU | E | 175 | −6.740 | 34.802 | 0.227 | 1.00 | 63.90 | C |
| ATOM | 10641 | C | LEU | E | 175 | −4.913 | 31.468 | −2.318 | 1.00 | 53.91 | C |
| ATOM | 10642 | O | LEU | E | 175 | −4.766 | 31.925 | −3.448 | 1.00 | 61.99 | O |
| ATOM | 10643 | N | GLY | E | 176 | −3.908 | 31.093 | −1.553 | 1.00 | 40.02 | N |
| ATOM | 10645 | CA | GLY | E | 176 | −2.533 | 31.180 | −1.943 | 1.00 | 38.23 | C |
| ATOM | 10648 | C | GLY | E | 176 | −1.601 | 30.964 | −0.754 | 1.00 | 46.69 | C |
| ATOM | 10649 | O | GLY | E | 176 | −1.877 | 30.271 | 0.251 | 1.00 | 45.82 | O |
| ATOM | 10650 | N | ASN | E | 177 | −0.439 | 31.585 | −0.869 | 1.00 | 44.89 | N |
| ATOM | 10652 | CA | ASN | E | 177 | 0.468 | 31.594 | 0.256 | 1.00 | 45.70 | C |
| ATOM | 10654 | CB | ASN | E | 177 | 1.258 | 30.297 | 0.274 | 1.00 | 39.35 | C |
| ATOM | 10657 | CG | ASN | E | 177 | 2.252 | 30.255 | −0.835 | 1.00 | 40.13 | C |
| ATOM | 10658 | OD1 | ASN | E | 177 | 3.186 | 31.055 | −0.872 | 1.00 | 57.00 | O |
| ATOM | 10659 | ND2 | ASN | E | 177 | 1.997 | 29.407 | −1.812 | 1.00 | 44.96 | N |
| ATOM | 10662 | C | ASN | E | 177 | 1.381 | 32.791 | 0.067 | 1.00 | 42.53 | C |
| ATOM | 10663 | O | ASN | E | 177 | 1.320 | 33.423 | −0.981 | 1.00 | 42.71 | O |
| ATOM | 10664 | N | GLY | E | 178 | 2.183 | 33.085 | 1.089 | 1.00 | 46.96 | N |
| ATOM | 10666 | CA | GLY | E | 178 | 2.984 | 34.292 | 1.183 | 1.00 | 48.78 | C |
| ATOM | 10669 | C | GLY | E | 178 | 4.213 | 34.221 | 2.094 | 1.00 | 51.00 | C |
| ATOM | 10670 | O | GLY | E | 178 | 4.281 | 33.514 | 3.104 | 1.00 | 41.02 | O |
| ATOM | 10671 | N | ASN | E | 179 | 5.171 | 35.076 | 1.763 | 1.00 | 53.93 | N |
| ATOM | 10673 | CA | ASN | E | 179 | 6.493 | 35.089 | 2.372 | 1.00 | 50.20 | C |
| ATOM | 10675 | CB | ASN | E | 179 | 7.501 | 34.447 | 1.437 | 1.00 | 54.24 | C |
| ATOM | 10678 | CG | ASN | E | 179 | 7.798 | 33.065 | 1.837 | 1.00 | 60.84 | C |
| ATOM | 10679 | OD1 | ASN | E | 179 | 8.697 | 32.842 | 2.636 | 1.00 | 93.24 | O |
| ATOM | 10680 | ND2 | ASN | E | 179 | 6.984 | 32.127 | 1.380 | 1.00 | 54.75 | N |
| ATOM | 10683 | C | ASN | E | 179 | 6.933 | 36.514 | 2.481 | 1.00 | 45.86 | C |
| ATOM | 10684 | O | ASN | E | 179 | 7.129 | 37.175 | 1.446 | 1.00 | 38.92 | O |
| ATOM | 10685 | N | ILE | E | 180 | 7.136 | 36.949 | 3.720 | 1.00 | 38.42 | N |
| ATOM | 10687 | CA | ILE | E | 180 | 7.633 | 38.279 | 3.980 | 1.00 | 35.68 | C |
| ATOM | 10689 | CB | ILE | E | 180 | 6.725 | 38.969 | 4.993 | 1.00 | 40.27 | C |
| ATOM | 10691 | CG1 | ILE | E | 180 | 5.330 | 39.155 | 4.393 | 1.00 | 49.34 | C |
| ATOM | 10694 | CD1 | ILE | E | 180 | 4.276 | 39.438 | 5.423 | 1.00 | 50.02 | C |
| ATOM | 10698 | CG2 | ILE | E | 180 | 7.286 | 40.329 | 5.343 | 1.00 | 42.46 | C |
| ATOM | 10702 | C | ILE | E | 180 | 9.036 | 38.153 | 4.524 | 1.00 | 38.09 | C |
| ATOM | 10703 | O | ILE | E | 180 | 9.250 | 37.465 | 5.533 | 1.00 | 34.92 | O |
| ATOM | 10704 | N | LYS | E | 181 | 9.980 | 38.812 | 3.841 | 1.00 | 45.90 | N |
| ATOM | 10706 | CA | LYS | E | 181 | 11.425 | 38.760 | 4.189 | 1.00 | 52.05 | C |
| ATOM | 10708 | CB | LYS | E | 181 | 12.240 | 38.207 | 3.006 | 1.00 | 53.73 | C |
| ATOM | 10711 | CG | LYS | E | 181 | 13.326 | 37.198 | 3.385 | 1.00 | 80.34 | C |
| ATOM | 10714 | CD | LYS | E | 181 | 14.757 | 37.761 | 3.233 | 1.00 | 110.30 | C |
| ATOM | 10717 | CE | LYS | E | 181 | 15.527 | 37.843 | 4.563 | 1.00 | 115.38 | C |
| ATOM | 10720 | NZ | LYS | E | 181 | 15.718 | 36.517 | 5.240 | 1.00 | 121.59 | N |
| ATOM | 10724 | C | LYS | E | 181 | 12.062 | 40.054 | 4.752 | 1.00 | 43.35 | C |
| ATOM | 10725 | O | LYS | E | 181 | 12.037 | 41.125 | 4.154 | 1.00 | 35.64 | O |
| ATOM | 10726 | N | LEU | E | 182 | 12.585 | 39.985 | 5.966 | 1.00 | 51.90 | N |
| ATOM | 10728 | CA | LEU | E | 182 | 13.159 | 41.168 | 6.607 | 1.00 | 47.79 | C |
| ATOM | 10730 | CB | LEU | E | 182 | 12.486 | 41.421 | 7.942 | 1.00 | 46.69 | C |
| ATOM | 10733 | CG | LEU | E | 182 | 11.249 | 42.316 | 7.783 | 1.00 | 48.87 | C |
| ATOM | 10735 | CD1 | LEU | E | 182 | 10.524 | 42.330 | 9.097 | 1.00 | 53.79 | C |
| ATOM | 10739 | CD2 | LEU | E | 182 | 11.693 | 43.724 | 7.438 | 1.00 | 73.63 | C |
| ATOM | 10743 | C | LEU | E | 182 | 14.654 | 41.002 | 6.820 | 1.00 | 51.84 | C |
| ATOM | 10744 | O | LEU | E | 182 | 15.082 | 40.155 | 7.603 | 1.00 | 46.30 | O |
| ATOM | 10745 | N | SER | E | 183 | 15.435 | 41.806 | 6.100 | 1.00 | 59.84 | N |
| ATOM | 10747 | CA | SER | E | 183 | 16.879 | 41.882 | 6.314 | 1.00 | 64.91 | C |
| ATOM | 10749 | CB | SER | E | 183 | 17.592 | 42.486 | 5.117 | 1.00 | 64.89 | C |
| ATOM | 10752 | OG | SER | E | 183 | 17.707 | 41.506 | 4.100 | 1.00 | 91.20 | O |
| ATOM | 10754 | C | SER | E | 183 | 17.295 | 42.639 | 7.563 | 1.00 | 63.21 | C |
| ATOM | 10755 | O | SER | E | 183 | 16.649 | 43.613 | 8.000 | 1.00 | 60.40 | O |
| ATOM | 10756 | N | GLN | E | 184 | 18.347 | 42.082 | 8.155 | 1.00 | 60.21 | N |
| ATOM | 10758 | CA | GLN | E | 184 | 19.170 | 42.736 | 9.153 | 1.00 | 57.70 | C |
| ATOM | 10760 | CB | GLN | E | 184 | 20.215 | 41.738 | 9.635 | 1.00 | 51.95 | C |
| ATOM | 10763 | CG | GLN | E | 184 | 21.322 | 42.353 | 10.476 | 1.00 | 69.07 | C |
| ATOM | 10766 | CD | GLN | E | 184 | 21.655 | 41.551 | 11.724 | 1.00 | 67.26 | C |
| ATOM | 10767 | OE1 | GLN | E | 184 | 22.058 | 40.379 | 11.668 | 1.00 | 83.37 | O |
| ATOM | 10768 | NE2 | GLN | E | 184 | 21.521 | 42.203 | 12.860 | 1.00 | 59.33 | N |
| ATOM | 10771 | C | GLN | E | 184 | 19.805 | 43.975 | 8.559 | 1.00 | 57.67 | C |
| ATOM | 10772 | O | GLN | E | 184 | 20.260 | 43.944 | 7.419 | 1.00 | 63.52 | O |
| ATOM | 10773 | N | THR | E | 185 | 19.764 | 45.074 | 9.302 | 1.00 | 65.53 | N |
| ATOM | 10775 | CA | THR | E | 185 | 20.021 | 46.394 | 8.738 | 1.00 | 79.17 | C |
| ATOM | 10777 | CB | THR | E | 185 | 18.868 | 47.376 | 9.054 | 1.00 | 80.77 | C |
| ATOM | 10779 | OG1 | THR | E | 185 | 18.929 | 47.792 | 10.427 | 1.00 | 84.06 | O |
| ATOM | 10781 | CG2 | THR | E | 185 | 17.520 | 46.700 | 8.937 | 1.00 | 78.71 | C |
| ATOM | 10785 | C | THR | E | 185 | 21.310 | 46.939 | 9.330 | 1.00 | 89.21 | C |
| ATOM | 10786 | O | THR | E | 185 | 21.389 | 47.138 | 10.537 | 1.00 | 89.87 | O |
| ATOM | 10787 | N | SER | E | 186 | 22.309 | 47.179 | 8.482 | 1.00 | 106.44 | N |
| ATOM | 10789 | CA | SER | E | 186 | 23.606 | 47.692 | 8.935 | 1.00 | 118.28 | C |
| ATOM | 10791 | CB | SER | E | 186 | 24.791 | 46.918 | 8.318 | 1.00 | 119.11 | C |
| ATOM | 10794 | OG | SER | E | 186 | 24.696 | 46.783 | 6.907 | 1.00 | 120.66 | O |
| ATOM | 10796 | C | SER | E | 186 | 23.724 | 49.195 | 8.672 | 1.00 | 125.89 | C |
| ATOM | 10797 | O | SER | E | 186 | 23.955 | 49.963 | 9.617 | 1.00 | 123.85 | O |
| ATOM | 10798 | N | ASN | E | 187 | 23.507 | 49.570 | 7.404 | 1.00 | 135.62 | N |
| ATOM | 10800 | CA | ASN | E | 187 | 23.553 | 50.946 | 6.847 | 1.00 | 139.88 | C |
| ATOM | 10802 | CB | ASN | E | 187 | 22.234 | 51.725 | 7.072 | 1.00 | 138.50 | C |
| ATOM | 10805 | CG | ASN | E | 187 | 21.191 | 50.932 | 7.853 | 1.00 | 134.03 | C |
| ATOM | 10806 | OD1 | ASN | E | 187 | 20.892 | 51.249 | 9.014 | 1.00 | 121.96 | O |
| ATOM | 10807 | ND2 | ASN | E | 187 | 20.630 | 49.901 | 7.217 | 1.00 | 108.93 | N |
| ATOM | 10810 | C | ASN | E | 187 | 24.798 | 51.827 | 7.120 | 1.00 | 142.86 | C |
| ATOM | 10811 | O | ASN | E | 187 | 25.927 | 51.421 | 6.819 | 1.00 | 137.81 | O |
| ATOM | 10812 | N | VAL | E | 188 | 24.587 | 53.053 | 7.611 | 1.00 | 147.60 | N |
| ATOM | 10814 | CA | VAL | E | 188 | 25.315 | 53.574 | 8.780 | 1.00 | 148.07 | C |
| ATOM | 10816 | CB | VAL | E | 188 | 25.177 | 55.148 | 8.950 | 1.00 | 146.96 | C |
| ATOM | 10818 | CG1 | VAL | E | 188 | 28.555 | 55.839 | 8.883 | 1.00 | 141.81 | C |
| ATOM | 10822 | CG2 | VAL | E | 188 | 24.258 | 55.777 | 7.886 | 1.00 | 140.41 | C |
| ATOM | 10826 | C | VAL | E | 188 | 24.983 | 52.723 | 10.043 | 1.00 | 151.42 | C |
| ATOM | 10827 | O | VAL | E | 188 | 25.490 | 51.595 | 10.152 | 1.00 | 149.59 | O |
| ATOM | 10828 | N | ASP | E | 189 | 24.146 | 53.203 | 10.976 | 1.00 | 154.64 | N |
| ATOM | 10830 | CA | ASP | E | 189 | 23.652 | 52.368 | 12.094 | 1.00 | 154.31 | C |
| ATOM | 10832 | CB | ASP | E | 189 | 24.794 | 51.967 | 13.059 | 1.00 | 154.90 | C |
| ATOM | 10835 | CG | ASP | E | 189 | 25.712 | 53.138 | 13.460 | 1.00 | 150.67 | C |
| ATOM | 10836 | OD1 | ASP | E | 189 | 25.227 | 54.239 | 13.803 | 1.00 | 130.54 | O |
| ATOM | 10837 | OD2 | ASP | E | 189 | 26.961 | 53.032 | 13.514 | 1.00 | 153.55 | O |
| ATOM | 10838 | C | ASP | E | 189 | 22.467 | 52.904 | 12.929 | 1.00 | 153.11 | C |
| ATOM | 10839 | O | ASP | E | 189 | 22.508 | 52.835 | 14.169 | 1.00 | 152.41 | O |
| ATOM | 10840 | N | LYS | E | 190 | 21.405 | 53.388 | 12.272 | 1.00 | 149.05 | N |
| ATOM | 10842 | CA | LYS | E | 190 | 20.577 | 54.473 | 12.817 | 1.00 | 144.15 | C |
| ATOM | 10844 | CB | LYS | E | 190 | 20.385 | 55.579 | 11.772 | 1.00 | 142.86 | C |
| ATOM | 10847 | CG | LYS | E | 190 | 20.605 | 56.989 | 12.313 | 1.00 | 140.19 | C |
| ATOM | 10850 | CD | LYS | E | 190 | 19.578 | 57.363 | 13.379 | 1.00 | 131.76 | C |
| ATOM | 10853 | CE | LYS | E | 190 | 20.189 | 57.375 | 14.778 | 1.00 | 122.46 | C |
| ATOM | 10856 | NZ | LYS | E | 190 | 19.150 | 57.232 | 15.845 | 1.00 | 120.31 | N |
| ATOM | 10860 | C | LYS | E | 190 | 19.204 | 53.983 | 13.288 | 1.00 | 141.82 | C |
| ATOM | 10861 | O | LYS | E | 190 | 18.504 | 53.317 | 12.523 | 1.00 | 142.91 | O |
| ATOM | 10862 | N | GLU | E | 191 | 18.849 | 54.292 | 14.541 | 1.00 | 136.10 | N |
| ATOM | 10864 | CA | GLU | E | 191 | 17.467 | 54.508 | 15.006 | 1.00 | 127.93 | C |
| ATOM | 10866 | CB | GLU | E | 191 | 16.514 | 54.846 | 13.839 | 1.00 | 128.94 | C |
| ATOM | 10869 | CG | GLU | E | 191 | 15.821 | 56.213 | 13.896 | 1.00 | 131.16 | C |
| ATOM | 10872 | CD | GLU | E | 191 | 14.893 | 56.492 | 12.707 | 1.00 | 132.62 | C |
| ATOM | 10873 | OE1 | GLU | E | 191 | 14.301 | 57.592 | 12.655 | 1.00 | 125.34 | O |
| ATOM | 10874 | OE2 | GLU | E | 191 | 14.729 | 55.629 | 11.811 | 1.00 | 121.48 | O |
| ATOM | 10875 | C | GLU | E | 191 | 16.958 | 53.312 | 15.829 | 1.00 | 122.22 | C |
| ATOM | 10876 | O | GLU | E | 191 | 17.709 | 52.694 | 16.587 | 1.00 | 113.30 | O |
| ATOM | 10877 | N | GLU | E | 192 | 15.670 | 53.005 | 15.677 | 1.00 | 121.63 | N |
| ATOM | 10879 | CA | GLU | E | 192 | 14.914 | 52.144 | 16.594 | 1.00 | 116.95 | C |
| ATOM | 10881 | CB | GLU | E | 192 | 13.971 | 52.978 | 17.475 | 1.00 | 117.48 | C |
| ATOM | 10884 | CG | GLU | E | 192 | 14.561 | 53.362 | 18.830 | 1.00 | 125.65 | C |
| ATOM | 10887 | CD | GLU | E | 192 | 14.636 | 54.872 | 19.043 | 1.00 | 123.15 | C |
| ATOM | 10888 | OE1 | GLU | E | 192 | 14.735 | 55.306 | 20.216 | 1.00 | 107.56 | O |
| ATOM | 10889 | OE2 | GLU | E | 192 | 14.608 | 55.619 | 18.038 | 1.00 | 117.51 | O |
| ATOM | 10890 | C | GLU | E | 192 | 14.052 | 51.193 | 15.776 | 1.00 | 109.03 | C |
| ATOM | 10891 | O | GLU | E | 192 | 14.037 | 49.984 | 16.027 | 1.00 | 109.24 | O |
| ATOM | 10892 | N | GLU | E | 193 | 13.284 | 51.775 | 14.855 | 1.00 | 98.70 | N |
| ATOM | 10894 | CA | GLU | E | 193 | 13.355 | 51.471 | 13.410 | 1.00 | 95.04 | C |
| ATOM | 10896 | CB | GLU | E | 193 | 13.145 | 52.786 | 12.629 | 1.00 | 94.64 | C |
| ATOM | 10899 | CG | GLU | E | 193 | 12.997 | 52.692 | 11.109 | 1.00 | 105.73 | C |
| ATOM | 10902 | CD | GLU | E | 193 | 12.126 | 51.535 | 10.615 | 1.00 | 120.53 | C |
| ATOM | 10903 | OE1 | GLU | E | 193 | 12.682 | 50.600 | 9.983 | 1.00 | 111.36 | O |
| ATOM | 10904 | OE2 | GLU | E | 193 | 10.886 | 51.560 | 10.826 | 1.00 | 111.58 | O |
| ATOM | 10905 | C | GLU | E | 193 | 14.661 | 50.766 | 12.975 | 1.00 | 83.69 | C |
| ATOM | 10906 | O | GLU | E | 193 | 15.722 | 51.318 | 13.248 | 1.00 | 97.86 | O |
| ATOM | 10907 | N | ALA | E | 194 | 14.632 | 49.562 | 12.382 | 1.00 | 64.43 | N |
| ATOM | 10909 | CA | ALA | E | 194 | 15.775 | 48.625 | 12.511 | 1.00 | 58.35 | C |
| ATOM | 10911 | CB | ALA | E | 194 | 16.789 | 49.124 | 13.512 | 1.00 | 51.90 | C |
| ATOM | 10915 | C | ALA | E | 194 | 15.498 | 47.203 | 12.929 | 1.00 | 53.23 | C |
| ATOM | 10916 | O | ALA | E | 194 | 14.960 | 46.960 | 13.998 | 1.00 | 46.96 | O |
| ATOM | 10917 | N | VAL | E | 195 | 16.107 | 46.281 | 12.205 | 1.00 | 52.37 | N |
| ATOM | 10919 | CA | VAL | E | 195 | 16.106 | 44.888 | 12.586 | 1.00 | 49.86 | C |
| ATOM | 10921 | CB | VAL | E | 195 | 15.571 | 44.061 | 11.447 | 1.00 | 48.52 | C |
| ATOM | 10923 | CG1 | VAL | E | 195 | 15.604 | 42.593 | 11.820 | 1.00 | 56.66 | C |
| ATOM | 10927 | CG2 | VAL | E | 195 | 14.169 | 44.530 | 11.122 | 1.00 | 45.69 | C |
| ATOM | 10931 | C | VAL | E | 195 | 17.518 | 44.415 | 12.875 | 1.00 | 53.71 | C |
| ATOM | 10932 | O | VAL | E | 195 | 18.399 | 44.445 | 12.020 | 1.00 | 59.54 | O |
| ATOM | 10933 | N | THR | E | 196 | 17.729 | 43.962 | 14.098 | 1.00 | 51.89 | N |
| ATOM | 10935 | CA | THR | E | 196 | 18.941 | 43.252 | 14.430 | 1.00 | 48.53 | C |
| ATOM | 10937 | CB | THR | E | 196 | 19.667 | 44.054 | 15.498 | 1.00 | 48.73 | C |
| ATOM | 10939 | OG1 | THR | E | 196 | 18.802 | 44.247 | 16.610 | 1.00 | 52.26 | O |
| ATOM | 10941 | CG2 | THR | E | 196 | 19.913 | 45.476 | 15.030 | 1.00 | 61.91 | C |
| ATOM | 10945 | C | THR | E | 196 | 18.599 | 41.869 | 14.967 | 1.00 | 50.94 | C |
| ATOM | 10946 | O | THR | E | 196 | 17.806 | 41.715 | 15.887 | 1.00 | 62.61 | O |
| ATOM | 10947 | N | ILE | E | 197 | 19.257 | 40.864 | 14.424 | 1.00 | 47.50 | N |
| ATOM | 10949 | CA | ILE | E | 197 | 19.142 | 39.490 | 14.884 | 1.00 | 47.26 | C |
| ATOM | 10951 | CB | ILE | E | 197 | 19.029 | 38.625 | 13.619 | 1.00 | 47.56 | C |
| ATOM | 10953 | CG1 | ILE | E | 197 | 17.725 | 38.933 | 12.898 | 1.00 | 58.95 | C |
| ATOM | 10956 | CD1 | ILE | E | 197 | 17.952 | 39.658 | 11.606 | 1.00 | 78.47 | C |
| ATOM | 10960 | CG2 | ILE | E | 197 | 19.131 | 37.161 | 13.909 | 1.00 | 52.11 | C |
| ATOM | 10964 | C | ILE | E | 197 | 20.372 | 39.058 | 15.681 | 1.00 | 48.19 | C |
| ATOM | 10965 | O | ILE | E | 197 | 21.495 | 39.334 | 15.280 | 1.00 | 61.14 | O |
| ATOM | 10966 | N | GLU | E | 198 | 20.160 | 38.334 | 16.774 | 1.00 | 57.73 | N |
| ATOM | 10968 | CA | GLU | E | 198 | 21.220 | 37.653 | 17.524 | 1.00 | 61.39 | C |
| ATOM | 10970 | CB | GLU | E | 198 | 21.165 | 38.074 | 18.992 | 1.00 | 68.84 | C |
| ATOM | 10973 | CG | GLU | E | 198 | 21.099 | 39.583 | 19.184 | 1.00 | 83.81 | C |
| ATOM | 10976 | CD | GLU | E | 198 | 22.443 | 40.144 | 19.592 | 1.00 | 91.68 | C |
| ATOM | 10977 | OE1 | GLU | E | 198 | 23.452 | 39.576 | 19.122 | 1.00 | 88.34 | O |
| ATOM | 10978 | OE2 | GLU | E | 198 | 22.485 | 41.110 | 20.393 | 1.00 | 96.60 | O |
| ATOM | 10979 | C | GLU | E | 198 | 21.139 | 36.127 | 17.466 | 1.00 | 57.37 | C |
| ATOM | 10980 | O | GLU | E | 198 | 20.508 | 35.479 | 18.279 | 1.00 | 65.83 | O |
| ATOM | 10981 | N | MET | E | 199 | 21.849 | 35.541 | 16.526 | 1.00 | 61.54 | N |
| ATOM | 10983 | CA | MET | E | 199 | 21.707 | 34.131 | 16.209 | 1.00 | 65.25 | C |
| ATOM | 10985 | CB | MET | E | 199 | 21.976 | 33.996 | 14.708 | 1.00 | 66.38 | C |
| ATOM | 10988 | CG | MET | E | 199 | 21.060 | 33.033 | 14.003 | 1.00 | 72.27 | C |
| ATOM | 10991 | SD | MET | E | 199 | 22.078 | 31.711 | 13.378 | 1.00 | 66.04 | S |
| ATOM | 10992 | CE | MET | E | 199 | 22.185 | 32.120 | 11.602 | 1.00 | 84.14 | C |
| ATOM | 10996 | C | MET | E | 199 | 22.708 | 33.259 | 16.994 | 1.00 | 65.61 | C |
| ATOM | 10997 | O | MET | E | 199 | 23.830 | 33.669 | 17.266 | 1.00 | 56.25 | O |
| ATOM | 10998 | N | ASN | E | 200 | 22.317 | 32.046 | 17.364 | 1.00 | 68.10 | N |
| ATOM | 11000 | CA | ASN | E | 200 | 23.298 | 31.033 | 17.734 | 1.00 | 68.85 | C |
| ATOM | 11002 | CB | ASN | E | 200 | 23.129 | 30.560 | 19.182 | 1.00 | 61.13 | C |
| ATOM | 11005 | CG | ASN | E | 200 | 23.183 | 31.685 | 20.172 | 1.00 | 62.66 | C |
| ATOM | 11006 | OD1 | ASN | E | 200 | 23.399 | 32.835 | 19.814 | 1.00 | 88.51 | O |
| ATOM | 11007 | ND2 | ASN | E | 200 | 23.018 | 31.361 | 21.438 | 1.00 | 80.06 | N |
| ATOM | 11010 | C | ASN | E | 200 | 23.070 | 29.859 | 16.806 | 1.00 | 74.68 | C |
| ATOM | 11011 | O | ASN | E | 200 | 24.016 | 29.304 | 16.238 | 1.00 | 80.11 | O |
| ATOM | 11012 | N | GLU | E | 201 | 21.794 | 29.496 | 16.689 | 1.00 | 74.84 | N |
| ATOM | 11014 | CA | GLU | E | 201 | 21.324 | 28.562 | 15.679 | 1.00 | 72.21 | C |
| ATOM | 11016 | CB | GLU | E | 201 | 20.861 | 27.261 | 16.313 | 1.00 | 70.86 | C |
| ATOM | 11019 | CG | GLU | E | 201 | 21.957 | 26.626 | 17.113 | 1.00 | 66.06 | C |
| ATOM | 11022 | CD | GLU | E | 201 | 21.709 | 26.733 | 18.588 | 1.00 | 69.21 | C |
| ATOM | 11023 | OE1 | GLU | E | 201 | 21.710 | 25.663 | 19.232 | 1.00 | 95.71 | O |
| ATOM | 11024 | OE2 | GLU | E | 201 | 21.579 | 27.867 | 19.089 | 1.00 | 55.63 | O |
| ATOM | 11025 | C | GLU | E | 201 | 20.148 | 29.146 | 14.951 | 1.00 | 65.05 | C |
| ATOM | 11026 | O | GLU | E | 201 | 19.298 | 29.818 | 15.530 | 1.00 | 70.20 | O |
| ATOM | 11027 | N | PRO | E | 202 | 20.073 | 28.787 | 13.687 | 1.00 | 56.20 | N |
| ATOM | 11028 | CA | PRO | E | 202 | 18.865 | 28.969 | 12.910 | 1.00 | 57.21 | C |
| ATOM | 11030 | CB | PRO | E | 202 | 19.265 | 28.414 | 11.544 | 1.00 | 62.33 | C |
| ATOM | 11033 | CG | PRO | E | 202 | 20.693 | 28.692 | 11.504 | 1.00 | 55.22 | C |
| ATOM | 11036 | CD | PRO | E | 202 | 21.121 | 28.168 | 12.875 | 1.00 | 60.77 | C |
| ATOM | 11039 | C | PRO | E | 202 | 17.729 | 28.194 | 13.516 | 1.00 | 49.90 | C |
| ATOM | 11040 | O | PRO | E | 202 | 17.901 | 27.177 | 14.166 | 1.00 | 50.86 | O |
| ATOM | 11041 | N | VAL | E | 203 | 16.544 | 28.741 | 13.306 | 1.00 | 59.30 | N |
| ATOM | 11043 | CA | VAL | E | 203 | 15.310 | 28.251 | 13.903 | 1.00 | 56.50 | C |
| ATOM | 11045 | CB | VAL | E | 203 | 14.845 | 29.146 | 15.044 | 1.00 | 55.49 | C |
| ATOM | 11047 | CG1 | VAL | E | 203 | 13.368 | 28.991 | 15.250 | 1.00 | 72.85 | C |
| ATOM | 11051 | CG2 | VAL | E | 203 | 15.575 | 28.801 | 16.284 | 1.00 | 50.35 | C |
| ATOM | 11055 | C | VAL | E | 203 | 14.314 | 28.463 | 12.795 | 1.00 | 53.63 | C |
| ATOM | 11056 | O | VAL | E | 203 | 14.417 | 29.455 | 12.057 | 1.00 | 49.24 | O |
| ATOM | 11057 | N | GLN | E | 204 | 13.395 | 27.506 | 12.696 | 1.00 | 53.34 | N |
| ATOM | 11059 | CA | GLN | E | 204 | 12.239 | 27.553 | 11.801 | 1.00 | 54.36 | C |
| ATOM | 11061 | CB | GLN | E | 204 | 12.470 | 26.693 | 10.554 | 1.00 | 50.48 | C |
| ATOM | 11064 | CG | GLN | E | 204 | 11.333 | 26.665 | 9.535 | 1.00 | 59.27 | C |
| ATOM | 11067 | CD | GLN | E | 204 | 11.753 | 26.090 | 8.162 | 1.00 | 66.53 | C |
| ATOM | 11068 | OE1 | GLN | E | 204 | 12.402 | 26.759 | 7.353 | 1.00 | 64.62 | O |
| ATOM | 11069 | NE2 | GLN | E | 204 | 11.361 | 24.854 | 7.898 | 1.00 | 52.55 | N |
| ATOM | 11072 | C | GLN | E | 204 | 11.101 | 26.993 | 12.640 | 1.00 | 53.58 | C |
| ATOM | 11073 | O | GLN | E | 204 | 10.933 | 25.790 | 12.750 | 1.00 | 56.92 | O |
| ATOM | 11074 | N | LEU | E | 205 | 10.342 | 27.870 | 13.280 | 1.00 | 54.36 | N |
| ATOM | 11076 | CA | LEU | E | 205 | 9.151 | 27.431 | 13.983 | 1.00 | 52.53 | C |
| ATOM | 11078 | CB | LEU | E | 205 | 9.305 | 27.821 | 15.435 | 1.00 | 56.36 | C |
| ATOM | 11081 | CG | LEU | E | 205 | 10.568 | 27.211 | 15.997 | 1.00 | 61.58 | C |
| ATOM | 11083 | CD1 | LEU | E | 205 | 10.896 | 28.081 | 17.194 | 1.00 | 58.84 | C |
| ATOM | 11087 | CD2 | LEU | E | 205 | 10.301 | 25.733 | 16.352 | 1.00 | 74.74 | C |
| ATOM | 11091 | C | LEU | E | 205 | 7.814 | 27.976 | 13.480 | 1.00 | 45.45 | C |
| ATOM | 11092 | O | LEU | E | 205 | 7.740 | 29.038 | 12.859 | 1.00 | 38.24 | O |
| ATOM | 11093 | N | THR | E | 206 | 6.777 | 27.222 | 13.824 | 1.00 | 38.90 | N |
| ATOM | 11095 | CA | THR | E | 206 | 5.407 | 27.475 | 13.420 | 1.00 | 40.77 | C |
| ATOM | 11097 | CB | THR | E | 206 | 4.796 | 26.188 | 12.809 | 1.00 | 35.70 | C |
| ATOM | 11099 | OG1 | THR | E | 206 | 5.688 | 25.657 | 11.832 | 1.00 | 34.03 | O |
| ATOM | 11101 | CG2 | THR | E | 206 | 3.587 | 26.500 | 11.973 | 1.00 | 43.63 | C |
| ATOM | 11105 | C | THR | E | 206 | 4.580 | 27.910 | 14.633 | 1.00 | 41.43 | C |
| ATOM | 11106 | O | THR | E | 206 | 4.671 | 27.313 | 15.701 | 1.00 | 40.26 | O |
| ATOM | 11107 | N | PHE | E | 207 | 3.781 | 28.958 | 14.449 | 1.00 | 41.80 | N |
| ATOM | 11109 | CA | PHE | E | 207 | 2.946 | 29.506 | 15.499 | 1.00 | 41.09 | C |
| ATOM | 11111 | CB | PHE | E | 207 | 3.607 | 30.719 | 16.120 | 1.00 | 38.94 | C |
| ATOM | 11114 | CG | PHE | E | 207 | 4.927 | 30.420 | 16.765 | 1.00 | 43.85 | C |
| ATOM | 11115 | CD1 | PHE | E | 207 | 4.989 | 29.857 | 18.028 | 1.00 | 44.02 | C |
| ATOM | 11117 | CE1 | PHE | E | 207 | 6.205 | 29.569 | 18.614 | 1.00 | 41.71 | C |
| ATOM | 11119 | CZ | PHE | E | 207 | 7.382 | 29.867 | 17.950 | 1.00 | 42.43 | C |
| ATOM | 11121 | CE2 | PHE | E | 207 | 7.336 | 30.415 | 16.696 | 1.00 | 50.79 | C |
| ATOM | 11123 | CD2 | PHE | E | 207 | 6.111 | 30.711 | 16.112 | 1.00 | 50.88 | C |
| ATOM | 11125 | C | PHE | E | 207 | 1.659 | 29.976 | 14.854 | 1.00 | 46.62 | C |
| ATOM | 11126 | O | PHE | E | 207 | 1.629 | 30.313 | 13.665 | 1.00 | 46.08 | O |
| ATOM | 11127 | N | ALA | E | 208 | 0.645 | 30.043 | 15.706 | 1.00 | 43.16 | N |
| ATOM | 11129 | CA | ALA | E | 208 | −0.719 | 30.430 | 15.400 | 1.00 | 43.78 | C |
| ATOM | 11131 | CB | ALA | E | 208 | −1.573 | 29.828 | 16.486 | 1.00 | 48.26 | C |
| ATOM | 11135 | C | ALA | E | 208 | −1.006 | 31.945 | 15.334 | 1.00 | 45.67 | C |
| ATOM | 11136 | O | ALA | E | 208 | −0.917 | 32.671 | 16.331 | 1.00 | 39.54 | O |
| ATOM | 11137 | N | LEU | E | 209 | −1.442 | 32.440 | 14.182 | 1.00 | 46.89 | N |
| ATOM | 11139 | CA | LEU | E | 209 | −1.523 | 33.888 | 14.030 | 1.00 | 40.84 | C |
| ATOM | 11141 | CB | LEU | E | 209 | −1.763 | 34.236 | 12.583 | 1.00 | 38.46 | C |
| ATOM | 11144 | CG | LEU | E | 209 | −0.428 | 34.270 | 11.850 | 1.00 | 51.79 | C |
| ATOM | 11146 | CD1 | LEU | E | 209 | −0.579 | 33.705 | 10.454 | 1.00 | 62.73 | C |
| ATOM | 11150 | CD2 | LEU | E | 209 | 0.032 | 35.695 | 11.809 | 1.00 | 48.91 | C |
| ATOM | 11154 | C | LEU | E | 209 | −2.606 | 34.497 | 14.909 | 1.00 | 40.14 | C |
| ATOM | 11155 | O | LEU | E | 209 | −2.610 | 35.701 | 15.175 | 1.00 | 32.39 | O |
| ATOM | 11156 | N | ARG | E | 210 | −3.517 | 33.649 | 15.377 | 1.00 | 43.89 | N |
| ATOM | 11158 | CA | ARG | E | 210 | −4.747 | 34.106 | 16.019 | 1.00 | 43.75 | C |
| ATOM | 11160 | CB | ARG | E | 210 | −5.632 | 32.935 | 16.443 | 1.00 | 48.06 | C |
| ATOM | 11163 | CG | ARG | E | 210 | −7.093 | 33.256 | 16.586 | 1.00 | 68.84 | C |
| ATOM | 11166 | CD | ARG | E | 210 | −7.922 | 32.147 | 17.160 | 1.00 | 64.28 | C |
| ATOM | 11169 | NE | ARG | E | 210 | −7.996 | 32.266 | 18.616 | 1.00 | 74.42 | N |
| ATOM | 11171 | CZ | ARG | E | 210 | −8.962 | 32.894 | 19.298 | 1.00 | 76.71 | C |
| ATOM | 11172 | NH1 | ARG | E | 210 | −9.946 | 33.515 | 18.654 | 1.00 | 75.42 | N |
| ATOM | 11175 | NH2 | ARG | E | 210 | −8.957 | 32.885 | 20.633 | 1.00 | 53.59 | N |
| ATOM | 11178 | C | ARG | E | 210 | −4.241 | 34.811 | 17.235 | 1.00 | 42.17 | C |
| ATOM | 11179 | O | ARG | E | 210 | −4.673 | 35.927 | 17.527 | 1.00 | 43.62 | O |
| ATOM | 11180 | N | TYR | E | 211 | −3.249 | 34.166 | 17.850 | 1.00 | 43.28 | N |
| ATOM | 11182 | CA | TYR | E | 211 | −2.771 | 34.481 | 19.187 | 1.00 | 42.02 | C |
| ATOM | 11184 | CB | TYR | E | 211 | −2.084 | 33.257 | 19.780 | 1.00 | 42.62 | C |
| ATOM | 11187 | CG | TYR | E | 211 | −3.038 | 32.214 | 20.344 | 1.00 | 55.31 | C |
| ATOM | 11188 | CD1 | TYR | E | 211 | −3.434 | 31.103 | 19.593 | 1.00 | 58.19 | C |
| ATOM | 11190 | CE1 | TYR | E | 211 | −4.279 | 30.121 | 20.133 | 1.00 | 49.70 | C |
| ATOM | 11192 | CZ | TYR | E | 211 | −4.709 | 30.239 | 21.435 | 1.00 | 54.55 | C |
| ATOM | 11193 | OH | TYR | E | 211 | −5.510 | 29.281 | 22.013 | 1.00 | 73.08 | O |
| ATOM | 11195 | CE2 | TYR | E | 211 | −4.275 | 31.292 | 22.207 | 1.00 | 52.99 | C |
| ATOM | 11197 | CD2 | TYR | E | 211 | −3.465 | 32.274 | 21.664 | 1.00 | 55.23 | C |
| ATOM | 11199 | C | TYR | E | 211 | −1.764 | 35.629 | 19.056 | 1.00 | 43.24 | C |
| ATOM | 11200 | O | TYR | E | 211 | −1.832 | 36.615 | 19.792 | 1.00 | 47.46 | O |
| ATOM | 11201 | N | LEU | E | 212 | −0.877 | 35.577 | 18.067 | 1.00 | 39.21 | N |
| ATOM | 11203 | CA | LEU | E | 212 | 0.012 | 36.725 | 17.848 | 1.00 | 34.26 | C |
| ATOM | 11205 | CB | LEU | E | 212 | 0.975 | 36.487 | 16.690 | 1.00 | 30.90 | C |
| ATOM | 11208 | CG | LEU | E | 212 | 1.938 | 35.328 | 16.967 | 1.00 | 35.89 | C |
| ATOM | 11210 | CD1 | LEU | E | 212 | 2.546 | 34.863 | 15.657 | 1.00 | 37.11 | C |
| ATOM | 11214 | CD2 | LEU | E | 212 | 2.983 | 35.751 | 17.961 | 1.00 | 36.87 | C |
| ATOM | 11218 | C | LEU | E | 212 | −0.742 | 38.026 | 17.640 | 1.00 | 33.70 | C |
| ATOM | 11219 | O | LEU | E | 212 | −0.372 | 39.082 | 18.163 | 1.00 | 29.44 | O |
| ATOM | 11220 | N | ASN | E | 213 | −1.831 | 38.002 | 16.885 | 1.00 | 37.63 | N |
| ATOM | 11222 | CA | ASN | E | 213 | −2.524 | 39.275 | 16.733 | 1.00 | 29.07 | C |
| ATOM | 11224 | CB | ASN | E | 213 | −3.509 | 39.203 | 15.581 | 1.00 | 23.67 | C |
| ATOM | 11227 | CG | ASN | E | 213 | −2.848 | 39.283 | 14.220 | 1.00 | 24.72 | C |
| ATOM | 11228 | OD1 | ASN | E | 213 | −2.105 | 40.229 | 13.904 | 1.00 | 42.85 | O |
| ATOM | 11229 | ND2 | ASN | E | 213 | −3.171 | 38.317 | 13.369 | 1.00 | 36.38 | N |
| ATOM | 11232 | C | ASN | E | 213 | −3.191 | 39.756 | 18.020 | 1.00 | 30.37 | C |
| ATOM | 11233 | O | ASN | E | 213 | −3.506 | 40.931 | 18.127 | 1.00 | 43.12 | O |
| ATOM | 11234 | N | PHE | E | 214 | −3.423 | 38.894 | 19.010 | 1.00 | 37.38 | N |
| ATOM | 11236 | CA | PHE | E | 214 | −3.765 | 39.402 | 20.350 | 1.00 | 38.83 | C |
| ATOM | 11238 | CB | PHE | E | 214 | −4.259 | 38.319 | 21.323 | 1.00 | 44.29 | C |
| ATOM | 11241 | CG | PHE | E | 214 | −5.613 | 37.758 | 20.999 | 1.00 | 45.16 | C |
| ATOM | 11242 | CD1 | PHE | E | 214 | −6.735 | 38.540 | 21.101 | 1.00 | 60.74 | C |
| ATOM | 11244 | CE1 | PHE | E | 214 | −7.969 | 38.034 | 20.779 | 1.00 | 61.79 | C |
| ATOM | 11246 | CZ | PHE | E | 214 | −8.109 | 36.722 | 20.377 | 1.00 | 43.36 | C |
| ATOM | 11248 | CE2 | PHE | E | 214 | −7.030 | 35.907 | 20.377 | 1.00 | 36.07 | C |
| ATOM | 11250 | CD2 | PHE | E | 214 | −5.770 | 36.424 | 20.663 | 1.00 | 44.44 | C |
| ATOM | 11252 | C | PHE | E | 214 | −2.586 | 40.086 | 21.014 | 1.00 | 36.70 | C |
| ATOM | 11253 | O | PHE | E | 214 | −2.780 | 41.150 | 21.616 | 1.00 | 26.78 | O |
| ATOM | 11254 | N | PHE | E | 215 | −1.404 | 39.450 | 20.952 | 1.00 | 34.41 | N |
| ATOM | 11256 | CA | PHE | E | 215 | −0.182 | 40.015 | 21.553 | 1.00 | 31.92 | C |
| ATOM | 11258 | CB | PHE | E | 215 | 1.079 | 39.218 | 21.246 | 1.00 | 32.48 | C |
| ATOM | 11261 | CG | PHE | E | 215 | 1.049 | 37.805 | 21.736 | 1.00 | 34.98 | C |
| ATOM | 11262 | CD1 | PHE | E | 215 | 0.022 | 37.366 | 22.540 | 1.00 | 50.51 | C |
| ATOM | 11264 | CE1 | PHE | E | 215 | −0.053 | 36.048 | 22.944 | 1.00 | 40.11 | C |
| ATOM | 11266 | CZ | PHE | E | 215 | 0.886 | 35.164 | 22.535 | 1.00 | 34.33 | C |
| ATOM | 11268 | CE2 | PHE | E | 215 | 1.943 | 35.587 | 21.744 | 1.00 | 44.72 | C |
| ATOM | 11270 | CD2 | PHE | E | 215 | 2.008 | 36.898 | 21.328 | 1.00 | 33.82 | C |
| ATOM | 11272 | C | PHE | E | 215 | 0.084 | 41.414 | 21.064 | 1.00 | 30.25 | C |
| ATOM | 11273 | O | PHE | E | 215 | 0.492 | 42.244 | 21.850 | 1.00 | 36.46 | O |
| ATOM | 11274 | N | THR | E | 216 | −0.125 | 41.683 | 19.781 | 1.00 | 36.35 | N |
| ATOM | 11276 | CA | THR | E | 216 | 0.222 | 42.993 | 19.227 | 1.00 | 42.19 | C |
| ATOM | 11278 | CB | THR | E | 216 | 0.216 | 42.964 | 17.696 | 1.00 | 38.39 | C |
| ATOM | 11280 | OG1 | THR | E | 216 | −1.057 | 42.529 | 17.230 | 1.00 | 43.16 | O |
| ATOM | 11282 | CG2 | THR | E | 216 | 1.165 | 41.899 | 17.189 | 1.00 | 38.11 | C |
| ATOM | 11286 | C | THR | E | 216 | −0.656 | 44.140 | 19.727 | 1.00 | 41.62 | C |
| ATOM | 11287 | O | THR | E | 216 | −0.356 | 45.314 | 19.526 | 1.00 | 45.82 | O |
| ATOM | 11288 | N | LYS | E | 217 | −1.717 | 43.818 | 20.446 | 1.00 | 43.50 | N |
| ATOM | 11290 | CA | LYS | E | 217 | −2.491 | 44.881 | 21.080 | 1.00 | 43.22 | C |
| ATOM | 11292 | CB | LYS | E | 217 | −3.687 | 44.287 | 21.821 | 1.00 | 40.66 | C |
| ATOM | 11295 | CG | LYS | E | 217 | −5.025 | 44.610 | 21.161 | 1.00 | 50.97 | C |
| ATOM | 11298 | CD | LYS | E | 217 | −5.640 | 43.488 | 20.351 | 1.00 | 66.76 | C |
| ATOM | 11301 | CE | LYS | E | 217 | −5.060 | 43.467 | 18.917 | 1.00 | 92.14 | C |
| ATOM | 11304 | NZ | LYS | E | 217 | −5.312 | 44.698 | 18.078 | 1.00 | 80.82 | N |
| ATOM | 11308 | C | LYS | E | 217 | −1.638 | 45.771 | 21.991 | 1.00 | 42.17 | C |
| ATOM | 11309 | O | LYS | E | 217 | −2.071 | 46.851 | 22.414 | 1.00 | 35.71 | O |
| ATOM | 11310 | N | ALA | E | 218 | −0.393 | 45.353 | 22.215 | 1.00 | 39.45 | N |
| ATOM | 11312 | CA | ALA | E | 218 | 0.485 | 46.054 | 23.129 | 1.00 | 34.25 | C |
| ATOM | 11314 | CB | ALA | E | 218 | 1.339 | 45.065 | 23.838 | 1.00 | 42.56 | C |
| ATOM | 11318 | C | ALA | E | 218 | 1.343 | 47.068 | 22.398 | 1.00 | 35.79 | C |
| ATOM | 11319 | O | ALA | E | 218 | 2.232 | 47.692 | 22.970 | 1.00 | 48.14 | O |
| ATOM | 11320 | N | THR | E | 219 | 1.112 | 47.224 | 21.106 | 1.00 | 40.16 | N |
| ATOM | 11322 | CA | THR | E | 219 | 2.108 | 47.881 | 20.258 | 1.00 | 40.69 | C |
| ATOM | 11324 | CB | THR | E | 219 | 1.617 | 47.737 | 18.827 | 1.00 | 38.53 | C |
| ATOM | 11326 | OG1 | THR | E | 219 | 1.733 | 46.356 | 18.430 | 1.00 | 42.33 | O |
| ATOM | 11328 | CG2 | THR | E | 219 | 2.497 | 48.512 | 17.916 | 1.00 | 26.36 | C |
| ATOM | 11332 | C | THR | E | 219 | 2.307 | 49.357 | 20.646 | 1.00 | 38.11 | C |
| ATOM | 11333 | O | THR | E | 219 | 3.365 | 49.951 | 20.417 | 1.00 | 40.39 | O |
| ATOM | 11334 | N | PRO | E | 220 | 1.246 | 49.958 | 21.185 | 1.00 | 32.96 | N |
| ATOM | 11335 | CA | PRO | E | 220 | 1.331 | 51.303 | 21.737 | 1.00 | 27.33 | C |
| ATOM | 11337 | CB | PRO | E | 220 | −0.090 | 51.555 | 22.243 | 1.00 | 29.38 | C |
| ATOM | 11340 | CG | PRO | E | 220 | −0.932 | 50.801 | 21.241 | 1.00 | 19.79 | C |
| ATOM | 11343 | CD | PRO | E | 220 | −0.165 | 49.503 | 21.107 | 1.00 | 33.64 | C |
| ATOM | 11346 | C | PRO | E | 220 | 2.361 | 51.485 | 22.807 | 1.00 | 32.84 | C |
| ATOM | 11347 | O | PRO | E | 220 | 2.683 | 52.637 | 23.152 | 1.00 | 39.81 | O |
| ATOM | 11348 | N | LEU | E | 221 | 2.842 | 50.380 | 23.361 | 1.00 | 34.08 | N |
| ATOM | 11350 | CA | LEU | E | 221 | 3.633 | 50.487 | 24.590 | 1.00 | 37.61 | C |
| ATOM | 11352 | CB | LEU | E | 221 | 3.537 | 49.236 | 25.485 | 1.00 | 37.30 | C |
| ATOM | 11355 | CG | LEU | E | 221 | 2.334 | 49.112 | 26.421 | 1.00 | 34.23 | C |
| ATOM | 11357 | CD1 | LEU | E | 221 | 2.215 | 47.685 | 26.910 | 1.00 | 50.59 | C |
| ATOM | 11361 | CD2 | LEU | E | 221 | 2.454 | 50.035 | 27.580 | 1.00 | 47.66 | C |
| ATOM | 11365 | C | LEU | E | 221 | 5.090 | 50.741 | 24.287 | 1.00 | 32.21 | C |
| ATOM | 11366 | O | LEU | E | 221 | 5.851 | 50.950 | 25.218 | 1.00 | 37.92 | O |
| ATOM | 11367 | N | SER | E | 222 | 5.479 | 50.569 | 23.028 | 1.00 | 38.70 | N |
| ATOM | 11369 | CA | SER | E | 222 | 6.878 | 50.420 | 22.633 | 1.00 | 42.84 | C |
| ATOM | 11371 | CB | SER | E | 222 | 7.516 | 49.145 | 23.195 | 1.00 | 45.84 | C |
| ATOM | 11374 | OG | SER | E | 222 | 8.883 | 48.994 | 22.792 | 1.00 | 52.68 | O |
| ATOM | 11376 | C | SER | E | 222 | 7.035 | 50.389 | 21.115 | 1.00 | 48.62 | C |
| ATOM | 11377 | O | SER | E | 222 | 6.266 | 49.777 | 20.361 | 1.00 | 43.14 | O |
| ATOM | 11378 | N | SER | E | 223 | 8.088 | 51.058 | 20.670 | 1.00 | 47.46 | N |
| ATOM | 11380 | CA | SER | E | 223 | 8.215 | 51.285 | 19.261 | 1.00 | 44.37 | C |
| ATOM | 11382 | CB | SER | E | 223 | 8.950 | 52.587 | 18.985 | 1.00 | 38.01 | C |
| ATOM | 11385 | OG | SER | E | 223 | 10.045 | 52.645 | 19.854 | 1.00 | 62.87 | O |
| ATOM | 11387 | C | SER | E | 223 | 8.928 | 50.061 | 18.747 | 1.00 | 37.04 | C |
| ATOM | 11388 | O | SER | E | 223 | 8.997 | 49.825 | 17.552 | 1.00 | 40.59 | O |
| ATOM | 11389 | N | SER | E | 224 | 9.325 | 49.185 | 19.653 | 1.00 | 40.93 | N |
| ATOM | 11391 | CA | THR | E | 224 | 10.233 | 48.103 | 19.275 | 1.00 | 46.26 | C |
| ATOM | 11393 | CB | THR | E | 224 | 11.691 | 48.509 | 19.614 | 1.00 | 37.41 | C |
| ATOM | 11395 | OG1 | THR | E | 224 | 12.508 | 47.345 | 19.743 | 1.00 | 67.09 | O |
| ATOM | 11397 | CG2 | THR | E | 224 | 11.768 | 49.137 | 20.989 | 1.00 | 64.50 | C |
| ATOM | 11401 | C | THR | E | 224 | 9.752 | 46.836 | 19.990 | 1.00 | 41.29 | C |
| ATOM | 11402 | O | THR | E | 224 | 9.190 | 46.984 | 21.076 | 1.00 | 41.35 | O |
| ATOM | 11403 | N | VAL | E | 225 | 9.851 | 45.659 | 19.339 | 1.00 | 32.55 | N |
| ATOM | 11405 | CA | VAL | E | 225 | 9.471 | 44.359 | 19.932 | 1.00 | 23.02 | C |
| ATOM | 11407 | CB | VAL | E | 225 | 8.193 | 43.795 | 19.276 | 1.00 | 30.21 | C |
| ATOM | 11409 | CG1 | VAL | E | 225 | 8.410 | 43.389 | 17.825 | 1.00 | 26.04 | C |
| ATOM | 11413 | CG2 | VAL | E | 225 | 7.661 | 42.564 | 19.974 | 1.00 | 27.42 | C |
| ATOM | 11417 | C | VAL | E | 225 | 10.582 | 43.321 | 19.743 | 1.00 | 25.98 | C |
| ATOM | 11418 | O | VAL | E | 225 | 11.156 | 43.258 | 18.670 | 1.00 | 27.32 | O |
| ATOM | 11419 | N | THR | E | 226 | 10.920 | 42.528 | 20.762 | 1.00 | 26.78 | N |
| ATOM | 11421 | CA | THR | E | 226 | 11.867 | 41.431 | 20.563 | 1.00 | 34.12 | C |
| ATOM | 11423 | CB | THR | E | 226 | 13.034 | 41.521 | 21.586 | 1.00 | 31.85 | C |
| ATOM | 11425 | OG1 | THR | E | 226 | 12.540 | 41.264 | 22.900 | 1.00 | 50.92 | O |
| ATOM | 11427 | CG2 | THR | E | 226 | 13.495 | 42.934 | 21.736 | 1.00 | 43.01 | C |
| ATOM | 11431 | C | THR | E | 226 | 11.199 | 40.030 | 20.564 | 1.00 | 37.13 | C |
| ATOM | 11432 | O | THR | E | 226 | 10.392 | 39.701 | 21.439 | 1.00 | 40.60 | O |
| ATOM | 11433 | N | LEU | E | 227 | 11.522 | 39.209 | 19.568 | 1.00 | 34.33 | N |
| ATOM | 11435 | CA | LEU | E | 227 | 11.019 | 37.852 | 19.495 | 1.00 | 35.28 | C |
| ATOM | 11437 | CB | LEU | E | 227 | 10.566 | 37.548 | 18.068 | 1.00 | 44.80 | C |
| ATOM | 11440 | CG | LEU | E | 227 | 9.817 | 38.677 | 17.339 | 1.00 | 36.41 | C |
| ATOM | 11442 | CD1 | LEU | E | 227 | 9.752 | 38.319 | 15.875 | 1.00 | 44.85 | C |
| ATOM | 11446 | CD2 | LEU | E | 227 | 8.389 | 38.803 | 17.817 | 1.00 | 35.96 | C |
| ATOM | 11450 | C | LEU | E | 227 | 12.186 | 36.951 | 19.852 | 1.00 | 39.75 | C |
| ATOM | 11451 | O | LEU | E | 227 | 13.273 | 37.036 | 19.288 | 1.00 | 47.72 | O |
| ATOM | 11452 | N | SER | E | 228 | 11.989 | 36.102 | 20.843 | 1.00 | 44.42 | N |
| ATOM | 11454 | CA | SER | E | 228 | 13.045 | 35.197 | 21.267 | 1.00 | 37.02 | C |
| ATOM | 11456 | CB | SER | E | 228 | 13.431 | 35.533 | 22.724 | 1.00 | 45.42 | C |
| ATOM | 11459 | OG | SER | E | 228 | 13.078 | 36.880 | 23.096 | 1.00 | 38.36 | O |
| ATOM | 11461 | C | SER | E | 228 | 12.534 | 33.759 | 21.087 | 1.00 | 34.01 | C |
| ATOM | 11462 | O | SER | E | 228 | 11.464 | 33.372 | 21.553 | 1.00 | 27.49 | O |
| ATOM | 11463 | N | MET | E | 229 | 13.290 | 33.001 | 20.320 | 1.00 | 35.03 | N |
| ATOM | 11465 | CA | MET | E | 229 | 12.843 | 31.773 | 19.687 | 1.00 | 42.69 | C |
| ATOM | 11467 | CB | MET | E | 229 | 12.900 | 31.946 | 18.170 | 1.00 | 42.36 | C |
| ATOM | 11470 | CG | MET | E | 229 | 11.584 | 31.822 | 17.477 | 1.00 | 52.72 | C |
| ATOM | 11473 | SD | MET | E | 229 | 11.335 | 33.229 | 16.437 | 1.00 | 66.45 | S |
| ATOM | 11474 | CE | MET | E | 229 | 12.934 | 33.420 | 15.617 | 1.00 | 65.15 | C |
| ATOM | 11478 | C | MET | E | 229 | 13.923 | 30.738 | 20.001 | 1.00 | 48.90 | C |
| ATOM | 11479 | O | MET | E | 229 | 15.131 | 30.995 | 19.778 | 1.00 | 44.76 | O |
| ATOM | 11480 | N | SER | E | 230 | 13.468 | 29.557 | 20.419 | 1.00 | 42.83 | N |
| ATOM | 11482 | CA | SER | E | 230 | 14.257 | 28.344 | 20.296 | 1.00 | 42.02 | C |
| ATOM | 11484 | CB | SER | E | 230 | 14.962 | 28.067 | 21.623 | 1.00 | 46.61 | C |
| ATOM | 11487 | OG | SER | E | 230 | 16.045 | 28.968 | 21.824 | 1.00 | 60.22 | O |
| ATOM | 11489 | C | SER | E | 230 | 13.403 | 27.132 | 19.891 | 1.00 | 50.07 | C |
| ATOM | 11490 | O | SER | E | 230 | 12.173 | 27.051 | 20.096 | 1.00 | 43.97 | O |
| ATOM | 11491 | N | ALA | E | 231 | 14.081 | 26.156 | 19.308 | 1.00 | 50.65 | N |
| ATOM | 11493 | CA | ALA | E | 231 | 13.430 | 24.906 | 18.958 | 1.00 | 52.03 | C |
| ATOM | 11495 | CB | ALA | E | 231 | 14.479 | 23.958 | 18.447 | 1.00 | 49.03 | C |
| ATOM | 11499 | C | ALA | E | 231 | 12.702 | 24.341 | 20.175 | 1.00 | 51.98 | C |
| ATOM | 11500 | O | ALA | E | 231 | 13.253 | 24.357 | 21.273 | 1.00 | 55.65 | O |
| ATOM | 11501 | N | ASP | E | 232 | 11.450 | 23.930 | 20.010 | 1.00 | 58.74 | N |
| ATOM | 11503 | CA | ASP | E | 232 | 10.734 | 23.126 | 21.018 | 1.00 | 62.62 | C |
| ATOM | 11505 | CB | ASP | E | 232 | 11.344 | 21.713 | 21.111 | 1.00 | 66.15 | C |
| ATOM | 11508 | CG | ASP | E | 232 | 11.216 | 20.936 | 19.799 | 1.00 | 75.40 | C |
| ATOM | 11509 | OD1 | ASP | E | 232 | 11.364 | 21.554 | 18.723 | 1.00 | 92.49 | O |
| ATOM | 11510 | OD2 | ASP | E | 232 | 10.889 | 19.733 | 19.724 | 1.00 | 88.37 | O |
| ATOM | 11511 | C | ASP | E | 232 | 10.603 | 23.799 | 22.393 | 1.00 | 59.69 | C |
| ATOM | 11512 | O | ASP | E | 232 | 10.453 | 23.152 | 23.427 | 1.00 | 65.79 | O |
| ATOM | 11513 | N | VAL | E | 233 | 10.621 | 25.125 | 22.396 | 1.00 | 60.50 | N |
| ATOM | 11515 | CA | VAL | E | 233 | 10.046 | 25.898 | 23.488 | 1.00 | 54.62 | C |
| ATOM | 11517 | CB | VAL | E | 233 | 11.149 | 26.406 | 24.416 | 1.00 | 56.78 | C |
| ATOM | 11519 | CG1 | VAL | E | 233 | 12.175 | 25.316 | 24.647 | 1.00 | 56.17 | C |
| ATOM | 11523 | CG2 | VAL | E | 233 | 11.782 | 27.670 | 23.848 | 1.00 | 43.61 | C |
| ATOM | 11527 | C | VAL | E | 233 | 9.241 | 27.110 | 23.017 | 1.00 | 51.51 | C |
| ATOM | 11528 | O | VAL | E | 233 | 9.318 | 27.546 | 21.863 | 1.00 | 51.80 | O |
| ATOM | 11529 | N | PRO | E | 234 | 8.473 | 27.682 | 23.935 | 1.00 | 51.07 | N |
| ATOM | 11530 | CA | PRO | E | 234 | 7.544 | 28.759 | 23.568 | 1.00 | 48.69 | C |
| ATOM | 11532 | CB | PRO | E | 234 | 6.835 | 29.076 | 24.893 | 1.00 | 49.55 | C |
| ATOM | 11535 | CG | PRO | E | 234 | 7.011 | 27.809 | 25.740 | 1.00 | 41.83 | C |
| ATOM | 11538 | CD | PRO | E | 234 | 8.379 | 27.327 | 25.365 | 1.00 | 43.23 | C |
| ATOM | 11541 | C | PRO | E | 234 | 8.269 | 29.976 | 23.026 | 1.00 | 48.07 | C |
| ATOM | 11542 | O | PRO | E | 234 | 9.383 | 30.317 | 23.427 | 1.00 | 52.64 | O |
| ATOM | 11543 | N | LEU | E | 235 | 7.598 | 30.671 | 22.126 | 1.00 | 45.00 | N |
| ATOM | 11545 | CA | LEU | E | 235 | 8.099 | 31.955 | 21.661 | 1.00 | 40.75 | C |
| ATOM | 11547 | CB | LEU | E | 235 | 7.429 | 32.286 | 20.339 | 1.00 | 34.81 | C |
| ATOM | 11550 | CG | LEU | E | 235 | 7.366 | 33.762 | 19.975 | 1.00 | 43.27 | C |
| ATOM | 11552 | CD1 | LEU | E | 235 | 8.767 | 34.198 | 19.529 | 1.00 | 40.33 | C |
| ATOM | 11556 | CD2 | LEU | E | 235 | 6.273 | 34.010 | 18.885 | 1.00 | 22.50 | C |
| ATOM | 11560 | C | LEU | E | 235 | 7.835 | 33.053 | 22.668 | 1.00 | 42.10 | C |
| ATOM | 11561 | O | LEU | E | 235 | 6.740 | 33.137 | 23.253 | 1.00 | 49.14 | O |
| ATOM | 11562 | N | VAL | E | 236 | 8.826 | 33.910 | 22.886 | 1.00 | 41.07 | N |
| ATOM | 11564 | CA | VAL | E | 236 | 8.525 | 35.100 | 23.689 | 1.00 | 36.70 | C |
| ATOM | 11566 | CB | VAL | E | 236 | 9.619 | 35.572 | 24.647 | 1.00 | 29.38 | C |
| ATOM | 11568 | CG1 | VAL | E | 236 | 8.968 | 36.252 | 25.769 | 1.00 | 38.61 | C |
| ATOM | 11572 | CG2 | VAL | E | 236 | 10.499 | 34.491 | 25.171 | 1.00 | 30.70 | C |
| ATOM | 11576 | C | VAL | E | 236 | 8.363 | 36.281 | 22.791 | 1.00 | 27.43 | C |
| ATOM | 11577 | O | VAL | E | 236 | 9.215 | 36.534 | 21.953 | 1.00 | 36.71 | O |
| ATOM | 11578 | N | VAL | E | 237 | 7.344 | 37.073 | 23.070 | 1.00 | 32.89 | N |
| ATOM | 11580 | CA | VAL | E | 237 | 7.136 | 38.338 | 22.390 | 1.00 | 30.94 | C |
| ATOM | 11582 | CB | VAL | E | 237 | 5.779 | 38.353 | 21.628 | 1.00 | 27.75 | C |
| ATOM | 11584 | CG1 | VAL | E | 237 | 5.562 | 39.704 | 20.911 | 1.00 | 27.82 | C |
| ATOM | 11588 | CG2 | VAL | E | 237 | 5.763 | 37.220 | 20.568 | 1.00 | 35.24 | C |
| ATOM | 11592 | C | VAL | E | 237 | 7.184 | 39.430 | 23.439 | 1.00 | 33.71 | C |
| ATOM | 11593 | O | VAL | E | 237 | 6.255 | 39.565 | 24.257 | 1.00 | 33.12 | O |
| ATOM | 11594 | N | GLU | E | 238 | 8.244 | 40.231 | 23.367 | 1.00 | 30.52 | N |
| ATOM | 11596 | CA | GLU | E | 238 | 8.568 | 41.125 | 24.480 | 1.00 | 42.53 | C |
| ATOM | 11598 | CB | GLU | E | 238 | 10.010 | 40.948 | 24.958 | 1.00 | 44.00 | C |
| ATOM | 11601 | CG | GLU | E | 238 | 10.283 | 41.667 | 26.261 | 1.00 | 48.82 | C |
| ATOM | 11604 | CD | GLU | E | 238 | 11.745 | 41.662 | 26.646 | 1.00 | 59.18 | C |
| ATOM | 11605 | OE1 | GLU | E | 238 | 12.418 | 40.671 | 26.303 | 1.00 | 46.21 | O |
| ATOM | 11606 | OE2 | GLU | E | 238 | 12.197 | 42.650 | 27.281 | 1.00 | 76.89 | O |
| ATOM | 11607 | C | GLU | E | 238 | 8.388 | 42.572 | 24.117 | 1.00 | 37.55 | C |
| ATOM | 11608 | O | GLU | E | 238 | 9.035 | 43.003 | 23.163 | 1.00 | 43.88 | O |
| ATOM | 11609 | N | TYR | E | 239 | 7.537 | 43.283 | 24.865 | 1.00 | 32.71 | N |
| ATOM | 11611 | CA | TYR | E | 239 | 7.373 | 44.726 | 24.731 | 1.00 | 35.89 | C |
| ATOM | 11613 | CB | TYR | E | 239 | 5.900 | 45.121 | 24.465 | 1.00 | 43.07 | C |
| ATOM | 11616 | CG | TYR | E | 239 | 5.309 | 44.565 | 23.202 | 1.00 | 42.92 | C |
| ATOM | 11617 | CD1 | TYR | E | 239 | 4.837 | 43.260 | 23.155 | 1.00 | 47.76 | C |
| ATOM | 11619 | CE1 | TYR | E | 239 | 4.382 | 42.707 | 21.983 | 1.00 | 47.87 | C |
| ATOM | 11621 | CZ | TYR | E | 239 | 4.366 | 43.463 | 20.827 | 1.00 | 43.71 | C |
| ATOM | 11622 | OH | TYR | E | 239 | 3.890 | 42.916 | 19.644 | 1.00 | 30.80 | O |
| ATOM | 11624 | CE2 | TYR | E | 239 | 4.820 | 44.769 | 20.863 | 1.00 | 35.93 | C |
| ATOM | 11626 | CD2 | TYR | E | 239 | 5.294 | 45.312 | 22.043 | 1.00 | 29.41 | C |
| ATOM | 11628 | C | TYR | E | 239 | 7.877 | 45.424 | 26.007 | 1.00 | 37.15 | C |
| ATOM | 11629 | O | TYR | E | 239 | 7.336 | 45.226 | 27.091 | 1.00 | 32.93 | O |
| ATOM | 11630 | N | LYS | E | 240 | 8.864 | 46.306 | 25.870 | 1.00 | 38.54 | N |
| ATOM | 11632 | CA | LYS | E | 240 | 9.465 | 46.945 | 27.030 | 1.00 | 34.78 | C |
| ATOM | 11634 | CB | LYS | E | 240 | 10.930 | 47.335 | 26.791 | 1.00 | 40.30 | C |
| ATOM | 11637 | CG | LYS | E | 240 | 11.877 | 46.084 | 26.890 | 1.00 | 64.36 | C |
| ATOM | 11640 | CD | LYS | E | 240 | 13.392 | 46.332 | 26.760 | 1.00 | 79.29 | C |
| ATOM | 11643 | CE | LYS | E | 240 | 14.209 | 45.345 | 27.614 | 1.00 | 77.77 | C |
| ATOM | 11646 | NZ | LYS | E | 240 | 15.210 | 46.005 | 28.512 | 1.00 | 77.16 | N |
| ATOM | 11650 | C | LYS | E | 240 | 8.633 | 48.119 | 27.386 | 1.00 | 30.61 | C |
| ATOM | 11651 | O | LYS | E | 240 | 8.173 | 48.811 | 26.474 | 1.00 | 39.19 | O |
| ATOM | 11652 | N | ILE | E | 241 | 8.340 | 48.277 | 28.686 | 1.00 | 37.31 | N |
| ATOM | 11654 | CA | ILE | E | 241 | 7.524 | 49.403 | 29.170 | 1.00 | 36.25 | C |
| ATOM | 11656 | CB | ILE | E | 241 | 6.525 | 48.954 | 30.219 | 1.00 | 37.41 | C |
| ATOM | 11658 | CG1 | ILE | E | 241 | 5.706 | 47.822 | 29.610 | 1.00 | 42.31 | C |
| ATOM | 11661 | CD1 | ILE | E | 241 | 4.618 | 47.333 | 30.485 | 1.00 | 40.80 | C |
| ATOM | 11665 | CG2 | ILE | E | 241 | 5.542 | 50.092 | 30.551 | 1.00 | 36.78 | C |
| ATOM | 11669 | C | ILE | E | 241 | 8.372 | 50.538 | 29.675 | 1.00 | 37.68 | C |
| ATOM | 11670 | O | ILE | E | 241 | 8.748 | 50.548 | 30.823 | 1.00 | 42.50 | O |
| ATOM | 11671 | N | ALA | E | 242 | 8.822 | 51.386 | 28.757 | 1.00 | 52.63 | N |
| ATOM | 11673 | CA | ALA | E | 242 | 9.439 | 52.655 | 29.087 | 1.00 | 52.87 | C |
| ATOM | 11675 | CB | ALA | E | 242 | 8.350 | 53.719 | 29.272 | 1.00 | 60.58 | C |
| ATOM | 11679 | C | ALA | E | 242 | 10.199 | 52.535 | 30.368 | 1.00 | 55.22 | C |
| ATOM | 11680 | O | ALA | E | 242 | 9.770 | 53.072 | 31.392 | 1.00 | 53.65 | O |
| ATOM | 11681 | N | ASP | E | 243 | 11.339 | 51.872 | 30.320 | 1.00 | 60.28 | N |
| ATOM | 11683 | CA | ASP | E | 243 | 12.196 | 51.813 | 31.514 | 1.00 | 71.31 | C |
| ATOM | 11685 | CB | ASP | E | 243 | 13.269 | 52.907 | 31.469 | 1.00 | 75.23 | C |
| ATOM | 11688 | CG | ASP | E | 243 | 14.633 | 52.387 | 31.006 | 1.00 | 86.94 | C |
| ATOM | 11689 | OD1 | ASP | E | 243 | 14.929 | 51.190 | 31.234 | 1.00 | 95.51 | O |
| ATOM | 11690 | OD2 | ASP | E | 243 | 15.480 | 53.112 | 30.426 | 1.00 | 94.44 | O |
| ATOM | 11691 | C | ASP | E | 243 | 11.396 | 51.855 | 32.835 | 1.00 | 68.87 | C |
| ATOM | 11692 | O | ASP | E | 243 | 11.386 | 52.849 | 33.581 | 1.00 | 68.82 | O |
| ATOM | 11693 | N | MET | E | 244 | 10.720 | 50.733 | 33.077 | 1.00 | 62.64 | N |
| ATOM | 11695 | CA | MET | E | 244 | 9.926 | 50.475 | 34.267 | 1.00 | 50.78 | C |
| ATOM | 11697 | CB | MET | E | 244 | 8.620 | 51.225 | 34.161 | 1.00 | 45.97 | C |
| ATOM | 11700 | CG | MET | E | 244 | 7.699 | 50.978 | 35.308 | 1.00 | 58.40 | C |
| ATOM | 11703 | SD | MET | E | 244 | 6.203 | 51.849 | 34.950 | 1.00 | 54.32 | S |
| ATOM | 11704 | CE | MET | E | 244 | 6.953 | 53.189 | 34.069 | 1.00 | 50.10 | C |
| ATOM | 11708 | C | MET | E | 244 | 9.649 | 48.968 | 34.303 | 1.00 | 44.45 | C |
| ATOM | 11709 | O | MET | E | 244 | 9.554 | 48.382 | 35.359 | 1.00 | 44.16 | O |
| ATOM | 11710 | N | GLY | E | 245 | 9.578 | 48.318 | 33.151 | 1.00 | 44.67 | N |
| ATOM | 11712 | CA | GLY | E | 245 | 9.576 | 46.862 | 33.057 | 1.00 | 42.83 | C |
| ATOM | 11715 | C | GLY | E | 245 | 9.177 | 46.426 | 31.654 | 1.00 | 41.44 | C |
| ATOM | 11716 | O | GLY | E | 245 | 9.802 | 46.876 | 30.711 | 1.00 | 45.20 | O |
| ATOM | 11717 | N | HIS | E | 246 | 8.134 | 45.615 | 31.497 | 1.00 | 37.99 | N |
| ATOM | 11719 | CA | HIS | E | 246 | 7.864 | 44.919 | 30.240 | 1.00 | 36.88 | C |
| ATOM | 11721 | CB | HIS | E | 246 | 9.030 | 44.012 | 29.804 | 1.00 | 36.39 | C |
| ATOM | 11724 | CG | HIS | E | 246 | 9.636 | 43.190 | 30.902 | 1.00 | 57.18 | C |
| ATOM | 11725 | ND1 | HIS | E | 246 | 10.839 | 43.524 | 31.502 | 1.00 | 66.98 | N |
| ATOM | 11727 | CE1 | HIS | E | 246 | 11.155 | 42.593 | 32.384 | 1.00 | 89.20 | C |
| ATOM | 11729 | NE2 | HIS | E | 246 | 10.226 | 41.650 | 32.351 | 1.00 | 93.81 | N |
| ATOM | 11731 | CD2 | HIS | E | 246 | 9.258 | 42.002 | 31.438 | 1.00 | 59.55 | C |
| ATOM | 11733 | C | HIS | E | 246 | 6.605 | 44.077 | 30.304 | 1.00 | 36.39 | C |
| ATOM | 11734 | O | HIS | E | 246 | 6.284 | 43.470 | 31.312 | 1.00 | 38.14 | O |
| ATOM | 11735 | N | LEU | E | 247 | 5.955 | 43.947 | 29.159 | 1.00 | 44.61 | N |
| ATOM | 11737 | CA | LEU | E | 247 | 4.953 | 42.906 | 28.923 | 1.00 | 40.72 | C |
| ATOM | 11739 | CB | LEU | E | 247 | 3.786 | 43.551 | 28.175 | 1.00 | 46.44 | C |
| ATOM | 11742 | CG | LEU | E | 247 | 2.363 | 43.020 | 28.242 | 1.00 | 36.52 | C |
| ATOM | 11744 | CD1 | LEU | E | 247 | 2.042 | 42.602 | 29.644 | 1.00 | 45.93 | C |
| ATOM | 11748 | CD2 | LEU | E | 247 | 1.436 | 44.163 | 27.782 | 1.00 | 44.38 | C |
| ATOM | 11752 | C | LEU | E | 247 | 5.561 | 41.820 | 28.064 | 1.00 | 30.97 | C |
| ATOM | 11753 | O | LEU | E | 247 | 6.126 | 42.096 | 27.022 | 1.00 | 32.64 | O |
| ATOM | 11754 | N | LYS | E | 248 | 5.456 | 40.581 | 28.498 | 1.00 | 33.26 | N |
| ATOM | 11756 | CA | LYS | E | 248 | 5.997 | 39.477 | 27.721 | 1.00 | 35.21 | C |
| ATOM | 11758 | CB | LYS | E | 248 | 7.015 | 38.721 | 28.544 | 1.00 | 38.77 | C |
| ATOM | 11761 | CG | LYS | E | 248 | 8.311 | 39.446 | 28.673 | 1.00 | 42.05 | C |
| ATOM | 11764 | CD | LYS | E | 248 | 9.332 | 38.607 | 29.425 | 1.00 | 34.45 | C |
| ATOM | 11767 | CE | LYS | E | 248 | 10.718 | 39.288 | 29.275 | 1.00 | 52.12 | C |
| ATOM | 11770 | NZ | LYS | E | 248 | 11.810 | 38.607 | 30.017 | 1.00 | 43.40 | N |
| ATOM | 11774 | C | LYS | E | 248 | 4.915 | 38.467 | 27.416 | 1.00 | 38.83 | C |
| ATOM | 11775 | O | LYS | E | 248 | 4.276 | 37.991 | 28.344 | 1.00 | 37.28 | O |
| ATOM | 11776 | N | TYR | E | 249 | 4.765 | 38.099 | 26.144 | 1.00 | 40.16 | N |
| ATOM | 11778 | CA | TYR | E | 249 | 3.781 | 37.103 | 25.752 | 1.00 | 42.05 | C |
| ATOM | 11780 | CB | TYR | E | 249 | 2.921 | 37.585 | 24.573 | 1.00 | 47.51 | C |
| ATOM | 11783 | CG | TYR | E | 249 | 2.096 | 38.823 | 24.846 | 1.00 | 25.84 | C |
| ATOM | 11784 | CD1 | TYR | E | 249 | 0.854 | 38.729 | 25.407 | 1.00 | 19.66 | C |
| ATOM | 11786 | CE1 | TYR | E | 249 | 0.104 | 39.885 | 25.689 | 1.00 | 33.40 | C |
| ATOM | 11788 | CZ | TYR | E | 249 | 0.621 | 41.132 | 25.407 | 1.00 | 35.61 | C |
| ATOM | 11789 | OH | TYR | E | 249 | −0.087 | 42.284 | 25.648 | 1.00 | 46.42 | O |
| ATOM | 11791 | CE2 | TYR | E | 249 | 1.866 | 41.249 | 24.867 | 1.00 | 29.29 | C |
| ATOM | 11793 | CD2 | TYR | E | 249 | 2.604 | 40.094 | 24.612 | 1.00 | 40.31 | C |
| ATOM | 11795 | C | TYR | E | 249 | 4.496 | 35.837 | 25.355 | 1.00 | 37.80 | C |
| ATOM | 11796 | O | TYR | E | 249 | 5.292 | 35.822 | 24.445 | 1.00 | 40.89 | O |
| ATOM | 11797 | N | TYR | E | 250 | 4.198 | 34.742 | 26.018 | 1.00 | 41.47 | N |
| ATOM | 11799 | CA | TYR | E | 250 | 4.855 | 33.486 | 25.670 | 1.00 | 40.42 | C |
| ATOM | 11801 | CB | TYR | E | 250 | 5.123 | 32.719 | 26.969 | 1.00 | 32.40 | C |
| ATOM | 11804 | CG | TYR | E | 250 | 6.275 | 33.257 | 27.755 | 1.00 | 34.47 | C |
| ATOM | 11805 | CD1 | TYR | E | 250 | 6.143 | 34.385 | 28.528 | 1.00 | 45.53 | C |
| ATOM | 11807 | CE1 | TYR | E | 250 | 7.233 | 34.880 | 29.244 | 1.00 | 43.85 | C |
| ATOM | 11809 | CZ | TYR | E | 250 | 8.436 | 34.239 | 29.193 | 1.00 | 49.19 | C |
| ATOM | 11810 | OH | TYR | E | 250 | 9.470 | 34.782 | 29.907 | 1.00 | 51.89 | O |
| ATOM | 11812 | CE2 | TYR | E | 250 | 8.595 | 33.108 | 28.411 | 1.00 | 38.45 | C |
| ATOM | 11814 | CD2 | TYR | E | 250 | 7.534 | 32.652 | 27.685 | 1.00 | 40.75 | C |
| ATOM | 11816 | C | TYR | E | 250 | 3.846 | 32.729 | 24.813 | 1.00 | 39.31 | C |
| ATOM | 11817 | O | TYR | E | 250 | 2.656 | 32.744 | 25.150 | 1.00 | 39.87 | O |
| ATOM | 11818 | N | LEU | E | 251 | 4.282 | 32.064 | 23.744 | 1.00 | 39.37 | N |
| ATOM | 11820 | CA | LEU | E | 251 | 3.337 | 31.306 | 22.906 | 1.00 | 39.20 | C |
| ATOM | 11822 | CB | LEU | E | 251 | 3.045 | 32.050 | 21.598 | 1.00 | 39.45 | C |
| ATOM | 11825 | CG | LEU | E | 251 | 2.156 | 31.333 | 20.570 | 1.00 | 42.32 | C |
| ATOM | 11827 | CD1 | LEU | E | 251 | 0.713 | 31.192 | 21.066 | 1.00 | 34.25 | C |
| ATOM | 11831 | CD2 | LEU | E | 251 | 2.168 | 32.110 | 19.267 | 1.00 | 29.88 | C |
| ATOM | 11835 | C | LEU | E | 251 | 3.809 | 29.908 | 22.560 | 1.00 | 31.04 | C |
| ATOM | 11836 | O | LEU | E | 251 | 4.828 | 29.747 | 21.923 | 1.00 | 34.97 | O |
| ATOM | 11837 | N | ALA | E | 252 | 3.074 | 28.894 | 22.981 | 1.00 | 30.80 | N |
| ATOM | 11839 | CA | ALA | E | 252 | 3.358 | 27.510 | 22.553 | 1.00 | 37.57 | C |
| ATOM | 11841 | CB | ALA | E | 252 | 2.330 | 26.499 | 23.155 | 1.00 | 32.02 | C |
| ATOM | 11845 | C | ALA | E | 252 | 3.466 | 27.289 | 21.047 | 1.00 | 33.61 | C |
| ATOM | 11846 | O | ALA | E | 252 | 2.674 | 27.772 | 20.257 | 1.00 | 41.86 | O |
| ATOM | 11847 | N | PRO | E | 253 | 4.448 | 26.521 | 20.629 | 1.00 | 42.64 | N |
| ATOM | 11848 | CA | PRO | E | 253 | 4.584 | 26.267 | 19.197 | 1.00 | 50.19 | C |
| ATOM | 11850 | CB | PRO | E | 253 | 6.024 | 25.761 | 19.036 | 1.00 | 50.11 | C |
| ATOM | 11853 | CG | PRO | E | 253 | 6.426 | 25.212 | 20.395 | 1.00 | 50.62 | C |
| ATOM | 11856 | CD | PRO | E | 253 | 5.502 | 25.863 | 21.421 | 1.00 | 46.08 | C |
| ATOM | 11859 | C | PRO | E | 253 | 3.587 | 25.199 | 18.845 | 1.00 | 52.21 | C |
| ATOM | 11860 | O | PRO | E | 253 | 3.206 | 24.463 | 19.744 | 1.00 | 52.48 | O |
| ATOM | 11861 | N | LYS | E | 254 | 3.197 | 25.116 | 17.578 | 1.00 | 55.77 | N |
| ATOM | 11863 | CA | LYS | E | 254 | 2.713 | 23.865 | 17.016 | 1.00 | 55.14 | C |
| ATOM | 11865 | CB | LYS | E | 254 | 1.526 | 24.124 | 16.101 | 1.00 | 52.44 | C |
| ATOM | 11868 | CG | LYS | E | 254 | 1.501 | 25.463 | 15.453 | 1.00 | 56.18 | C |
| ATOM | 11871 | CD | LYS | E | 254 | 0.479 | 25.463 | 14.313 | 1.00 | 71.52 | C |
| ATOM | 11874 | CE | LYS | E | 254 | −0.885 | 24.923 | 14.696 | 1.00 | 52.45 | C |
| ATOM | 11877 | NZ | LYS | E | 254 | −1.911 | 25.891 | 14.177 | 1.00 | 75.28 | N |
| ATOM | 11881 | C | LYS | E | 254 | 3.779 | 23.117 | 16.258 | 1.00 | 52.11 | C |
| ATOM | 11882 | O | LYS | E | 254 | 4.300 | 23.648 | 15.293 | 1.00 | 56.69 | O |
| ATOM | 11883 | N | ILE | E | 255 | 4.042 | 21.878 | 16.669 | 1.00 | 63.83 | N |
| ATOM | 11885 | CA | ILE | E | 255 | 4.977 | 20.993 | 15.974 | 1.00 | 70.06 | C |
| ATOM | 11887 | CB | ILE | E | 255 | 6.163 | 20.655 | 16.906 | 1.00 | 71.53 | C |
| ATOM | 11889 | CG1 | ILE | E | 255 | 7.271 | 19.893 | 16.136 | 1.00 | 80.85 | C |
| ATOM | 11892 | CD1 | ILE | E | 255 | 8.089 | 20.747 | 15.116 | 1.00 | 60.30 | C |
| ATOM | 11896 | CG2 | ILE | E | 255 | 5.660 | 20.024 | 18.198 | 1.00 | 63.75 | C |
| ATOM | 11900 | C | ILE | E | 255 | 4.303 | 19.754 | 15.390 | 1.00 | 77.74 | C |
| ATOM | 11901 | O | ILE | E | 255 | 3.079 | 19.636 | 15.476 | 1.00 | 83.58 | O |
| ATOM | 11902 | N | GLU | E | 256 | 5.064 | 18.878 | 14.731 | 1.00 | 89.08 | N |
| ATOM | 11904 | CA | GLU | E | 256 | 4.465 | 17.758 | 13.994 | 1.00 | 98.87 | C |
| ATOM | 11906 | CB | GLU | E | 256 | 5.160 | 17.520 | 12.660 | 1.00 | 103.49 | C |
| ATOM | 11909 | CG | GLU | E | 256 | 6.663 | 17.340 | 12.761 | 1.00 | 109.10 | C |
| ATOM | 11912 | CD | GLU | E | 256 | 7.389 | 18.312 | 11.851 | 1.00 | 113.66 | C |
| ATOM | 11913 | OE1 | GLU | E | 256 | 8.458 | 18.819 | 12.263 | 1.00 | 110.25 | O |
| ATOM | 11914 | OE2 | GLU | E | 256 | 6.852 | 18.593 | 10.748 | 1.00 | 91.91 | O |
| ATOM | 11915 | C | GLU | E | 256 | 4.521 | 16.452 | 14.752 | 1.00 | 103.21 | C |
| ATOM | 11916 | O | GLU | E | 256 | 5.411 | 16.266 | 15.583 | 1.00 | 103.29 | O |
| ATOM | 11917 | N | ASP | E | 257 | 3.628 | 15.535 | 14.374 | 1.00 | 109.45 | N |
| ATOM | 11919 | CA | ASP | E | 257 | 2.788 | 14.786 | 15.315 | 1.00 | 112.48 | C |
| ATOM | 11921 | CB | ASP | E | 257 | 2.773 | 13.299 | 14.954 | 1.00 | 113.98 | C |
| ATOM | 11924 | CG | ASP | E | 257 | 4.169 | 12.753 | 14.675 | 1.00 | 112.85 | C |
| ATOM | 11925 | OD1 | ASP | E | 257 | 4.981 | 12.601 | 15.617 | 1.00 | 108.22 | O |
| ATOM | 11926 | OD2 | ASP | E | 257 | 4.547 | 12.465 | 13.523 | 1.00 | 114.38 | O |
| ATOM | 11927 | C | ASP | E | 257 | 3.262 | 14.938 | 16.760 | 1.00 | 112.58 | C |
| ATOM | 11928 | O | ASP | E | 257 | 3.208 | 13.989 | 17.544 | 1.00 | 114.05 | O |
| ATOM | 11929 | N | MET | G | 1 | −3.746 | 9.664 | 83.233 | 1.00 | 67.79 | N |
| ATOM | 11931 | CA | MET | G | 1 | −3.107 | 10.292 | 84.409 | 1.00 | 71.92 | C |
| ATOM | 11933 | CB | MET | G | 1 | −1.650 | 10.593 | 84.062 | 1.00 | 76.82 | C |
| ATOM | 11936 | CG | MET | G | 1 | −1.154 | 10.010 | 82.710 | 1.00 | 89.52 | C |
| ATOM | 11939 | SD | MET | G | 1 | −1.820 | 10.700 | 81.117 | 1.00 | 105.86 | S |
| ATOM | 11940 | CE | MET | G | 1 | −0.346 | 11.049 | 80.153 | 1.00 | 78.34 | C |
| ATOM | 11944 | C | MET | G | 1 | −3.914 | 11.550 | 84.770 | 1.00 | 70.94 | C |
| ATOM | 11945 | O | MET | G | 1 | −5.074 | 11.442 | 85.170 | 1.00 | 73.28 | O |
| ATOM | 11948 | N | PHE | G | 2 | −3.377 | 12.750 | 84.562 | 1.00 | 66.13 | N |
| ATOM | 11950 | CA | PHE | G | 2 | −4.236 | 13.874 | 84.181 | 1.00 | 59.81 | C |
| ATOM | 11952 | CB | PHE | G | 2 | −3.944 | 15.082 | 85.023 | 1.00 | 55.46 | C |
| ATOM | 11955 | CG | PHE | G | 2 | −4.713 | 16.292 | 84.620 | 1.00 | 53.19 | C |
| ATOM | 11956 | CD1 | PHE | G | 2 | −6.082 | 16.317 | 84.686 | 1.00 | 53.71 | C |
| ATOM | 11958 | CE1 | PHE | G | 2 | −6.800 | 17.486 | 84.394 | 1.00 | 55.05 | C |
| ATOM | 11960 | CZ | PHE | G | 2 | −6.137 | 18.624 | 84.038 | 1.00 | 49.82 | C |
| ATOM | 11962 | CE2 | PHE | G | 2 | −4.766 | 18.613 | 83.994 | 1.00 | 57.33 | C |
| ATOM | 11964 | CD2 | PHE | G | 2 | −4.058 | 17.463 | 84.310 | 1.00 | 60.36 | C |
| ATOM | 11966 | C | PHE | G | 2 | −4.081 | 14.283 | 82.739 | 1.00 | 55.58 | C |
| ATOM | 11967 | O | PHE | G | 2 | −2.970 | 14.340 | 82.235 | 1.00 | 54.78 | O |
| ATOM | 11968 | N | GLU | G | 3 | −5.199 | 14.531 | 82.069 | 1.00 | 55.29 | N |
| ATOM | 11970 | CA | GLU | G | 3 | −5.155 | 15.055 | 80.711 | 1.00 | 54.20 | C |
| ATOM | 11972 | CB | GLU | G | 3 | −4.692 | 13.962 | 79.769 | 1.00 | 52.40 | C |
| ATOM | 11975 | CG | GLU | G | 3 | −4.220 | 14.467 | 78.424 | 1.00 | 63.83 | C |
| ATOM | 11978 | CD | GLU | G | 3 | −3.836 | 13.311 | 77.542 | 1.00 | 74.14 | C |
| ATOM | 11979 | OE1 | GLU | G | 3 | −3.003 | 13.478 | 76.622 | 1.00 | 60.66 | O |
| ATOM | 11980 | OE2 | GLU | G | 3 | −4.362 | 12.218 | 77.837 | 1.00 | 93.91 | O |
| ATOM | 11981 | C | GLU | G | 3 | −6.489 | 15.669 | 80.256 | 1.00 | 56.68 | C |
| ATOM | 11982 | O | GLU | G | 3 | −7.523 | 15.006 | 80.189 | 1.00 | 62.41 | O |
| ATOM | 11983 | N | ALA | G | 4 | −6.453 | 16.975 | 80.014 | 1.00 | 56.52 | N |
| ATOM | 11985 | CA | ALA | G | 4 | −7.607 | 17.767 | 79.614 | 1.00 | 51.37 | C |
| ATOM | 11987 | CB | ALA | G | 4 | −7.915 | 18.837 | 80.654 | 1.00 | 52.41 | C |
| ATOM | 11991 | C | ALA | G | 4 | −7.149 | 18.419 | 78.329 | 1.00 | 47.18 | C |
| ATOM | 11992 | O | ALA | G | 4 | −6.006 | 18.823 | 78.191 | 1.00 | 56.12 | O |
| ATOM | 11993 | N | ARG | G | 5 | −8.046 | 18.479 | 77.372 | 1.00 | 48.22 | N |
| ATOM | 11995 | CA | ARG | G | 5 | −7.788 | 19.070 | 76.069 | 1.00 | 44.82 | C |
| ATOM | 11997 | CB | ARG | G | 5 | −8.057 | 18.011 | 75.004 | 1.00 | 45.69 | C |
| ATOM | 12000 | CG | ARG | G | 5 | −7.803 | 18.447 | 73.609 | 1.00 | 48.01 | C |
| ATOM | 12003 | CD | ARG | G | 5 | −7.997 | 17.303 | 72.647 | 1.00 | 51.93 | C |
| ATOM | 12006 | NE | ARG | G | 5 | −8.607 | 17.741 | 71.400 | 1.00 | 49.57 | N |
| ATOM | 12008 | CZ | ARG | G | 5 | −7.903 | 17.963 | 70.305 | 1.00 | 66.88 | C |
| ATOM | 12009 | NH1 | ARG | G | 5 | −6.589 | 17.743 | 70.329 | 1.00 | 65.49 | N |
| ATOM | 12012 | NH2 | ARG | G | 5 | −8.508 | 18.388 | 69.200 | 1.00 | 70.88 | N |
| ATOM | 12015 | C | ARG | G | 5 | −8.820 | 20.153 | 75.904 | 1.00 | 38.75 | C |
| ATOM | 12016 | O | ARG | G | 5 | −9.999 | 19.961 | 76.169 | 1.00 | 34.51 | O |
| ATOM | 12017 | N | LEU | G | 6 | −8.380 | 21.318 | 75.495 | 1.00 | 41.51 | N |
| ATOM | 12019 | CA | LEU | G | 6 | −9.264 | 22.471 | 75.505 | 1.00 | 45.35 | C |
| ATOM | 12021 | CB | LEU | G | 6 | −8.860 | 23.455 | 76.594 | 1.00 | 43.23 | C |
| ATOM | 12024 | CG | LEU | G | 6 | −9.963 | 24.473 | 76.820 | 1.00 | 59.38 | C |
| ATOM | 12026 | CD1 | LEU | G | 6 | −10.907 | 23.840 | 77.820 | 1.00 | 71.69 | C |
| ATOM | 12030 | CD2 | LEU | G | 6 | −9.422 | 25.827 | 77.302 | 1.00 | 63.68 | C |
| ATOM | 12034 | C | LEU | G | 6 | −9.101 | 23.112 | 74.154 | 1.00 | 45.68 | C |
| ATOM | 12035 | O | LEU | G | 6 | −7.981 | 23.319 | 73.707 | 1.00 | 61.06 | O |
| ATOM | 12036 | N | VAL | G | 7 | −10.197 | 23.313 | 73.440 | 1.00 | 52.47 | N |
| ATOM | 12038 | CA | VAL | G | 7 | −10.105 | 23.618 | 72.019 | 1.00 | 50.53 | C |
| ATOM | 12040 | CB | VAL | G | 7 | −11.390 | 23.162 | 71.280 | 1.00 | 50.29 | C |
| ATOM | 12042 | CG1 | VAL | G | 7 | −11.302 | 23.432 | 69.803 | 1.00 | 56.71 | C |
| ATOM | 12046 | CG2 | VAL | G | 7 | −11.579 | 21.691 | 71.479 | 1.00 | 56.03 | C |
| ATOM | 12050 | C | VAL | G | 7 | −9.929 | 25.128 | 71.997 | 1.00 | 51.07 | C |
| ATOM | 12051 | O | VAL | G | 7 | −9.093 | 25.669 | 71.269 | 1.00 | 58.20 | O |
| ATOM | 12052 | N | GLN | G | 8 | −10.721 | 25.772 | 72.846 | 1.00 | 45.43 | N |
| ATOM | 12054 | CA | GLN | G | 8 | −10.664 | 27.197 | 73.055 | 1.00 | 51.09 | C |
| ATOM | 12056 | CB | GLN | G | 8 | −11.990 | 27.711 | 73.603 | 1.00 | 54.40 | C |
| ATOM | 12059 | CG | GLN | G | 8 | −13.015 | 28.032 | 72.550 | 1.00 | 70.04 | C |
| ATOM | 12062 | CD | GLN | G | 8 | −13.224 | 29.514 | 72.458 | 1.00 | 99.59 | C |
| ATOM | 12063 | OE1 | GLN | G | 8 | −14.038 | 30.080 | 73.204 | 1.00 | 109.44 | O |
| ATOM | 12064 | NE2 | GLN | G | 8 | −12.442 | 30.168 | 71.594 | 1.00 | 105.60 | N |
| ATOM | 12067 | C | GLN | G | 8 | −9.575 | 27.459 | 74.069 | 1.00 | 52.31 | C |
| ATOM | 12068 | O | GLN | G | 8 | −9.842 | 27.799 | 75.228 | 1.00 | 54.36 | O |
| ATOM | 12069 | N | GLY | G | 9 | −8.332 | 27.375 | 73.610 | 1.00 | 51.09 | N |
| ATOM | 12071 | CA | GLY | G | 9 | −7.220 | 27.722 | 74.476 | 1.00 | 48.21 | C |
| ATOM | 12074 | C | GLY | G | 9 | −7.238 | 29.148 | 74.968 | 1.00 | 43.85 | C |
| ATOM | 12075 | O | GLY | G | 9 | −6.649 | 29.439 | 76.005 | 1.00 | 50.01 | O |
| ATOM | 12076 | N | SER | G | 10 | −7.923 | 30.018 | 74.233 | 1.00 | 38.39 | N |
| ATOM | 12078 | CA | SER | G | 10 | −8.241 | 31.359 | 74.688 | 1.00 | 34.62 | C |
| ATOM | 12080 | CB | SER | G | 10 | −9.326 | 31.960 | 73.765 | 1.00 | 39.74 | C |
| ATOM | 12083 | OG | SER | G | 10 | −10.625 | 32.170 | 74.350 | 1.00 | 55.92 | O |
| ATOM | 12085 | C | SER | G | 10 | −8.620 | 31.421 | 76.173 | 1.00 | 37.79 | C |
| ATOM | 12086 | O | SER | G | 10 | −8.257 | 32.368 | 76.876 | 1.00 | 36.42 | O |
| ATOM | 12087 | N | ILE | G | 11 | −9.427 | 30.459 | 76.621 | 1.00 | 45.08 | N |
| ATOM | 12089 | CA | ILE | G | 11 | −10.101 | 30.540 | 77.909 | 1.00 | 45.24 | C |
| ATOM | 12091 | CB | ILE | G | 11 | −11.030 | 29.319 | 78.093 | 1.00 | 50.89 | C |
| ATOM | 12093 | CG1 | ILE | G | 11 | −12.354 | 29.561 | 77.390 | 1.00 | 55.81 | C |
| ATOM | 12096 | CD1 | ILE | G | 11 | −13.098 | 28.272 | 77.165 | 1.00 | 56.76 | C |
| ATOM | 12100 | CG2 | ILE | G | 11 | −11.394 | 29.076 | 79.568 | 1.00 | 48.90 | C |
| ATOM | 12104 | C | ILE | G | 11 | −9.016 | 30.492 | 78.966 | 1.00 | 43.74 | C |
| ATOM | 12105 | O | ILE | G | 11 | −8.906 | 31.359 | 79.826 | 1.00 | 45.54 | O |
| ATOM | 12106 | N | LEU | G | 12 | −8.201 | 29.457 | 78.878 | 1.00 | 47.56 | N |
| ATOM | 12108 | CA | LEU | G | 12 | −7.132 | 29.262 | 79.838 | 1.00 | 52.39 | C |
| ATOM | 12110 | CB | LEU | G | 12 | −6.363 | 27.996 | 79.495 | 1.00 | 52.22 | C |
| ATOM | 12113 | CG | LEU | G | 12 | −5.666 | 27.295 | 80.651 | 1.00 | 67.25 | C |
| ATOM | 12115 | CD1 | LEU | G | 12 | −6.552 | 27.131 | 81.881 | 1.00 | 64.44 | C |
| ATOM | 12119 | CD2 | LEU | G | 12 | −5.220 | 25.947 | 80.094 | 1.00 | 83.58 | C |
| ATOM | 12123 | C | LEU | G | 12 | −6.205 | 30.466 | 79.866 | 1.00 | 47.19 | C |
| ATOM | 12124 | O | LEU | G | 12 | −5.875 | 30.952 | 80.936 | 1.00 | 55.22 | O |
| ATOM | 12125 | N | LYS | G | 13 | −5.810 | 30.965 | 78.701 | 1.00 | 47.08 | N |
| ATOM | 12127 | CA | LYS | G | 13 | −5.090 | 32.236 | 78.616 | 1.00 | 36.27 | C |
| ATOM | 12129 | CB | LYS | G | 13 | −4.996 | 32.670 | 77.155 | 1.00 | 28.59 | C |
| ATOM | 12132 | CG | LYS | G | 13 | −3.782 | 32.130 | 76.418 | 1.00 | 37.60 | C |
| ATOM | 12135 | CD | LYS | G | 13 | −3.956 | 32.218 | 74.908 | 1.00 | 40.23 | C |
| ATOM | 12138 | CE | LYS | G | 13 | −3.385 | 33.502 | 74.321 | 1.00 | 39.49 | C |
| ATOM | 12141 | NZ | LYS | G | 13 | −1.932 | 33.652 | 74.552 | 1.00 | 40.98 | N |
| ATOM | 12145 | C | LYS | G | 13 | −5.741 | 33.336 | 79.454 | 1.00 | 41.08 | C |
| ATOM | 12146 | O | LYS | G | 13 | −5.107 | 33.923 | 80.349 | 1.00 | 42.99 | O |
| ATOM | 12147 | N | LYS | G | 14 | −7.010 | 33.620 | 79.171 | 1.00 | 46.70 | N |
| ATOM | 12149 | CA | LYS | G | 14 | −7.694 | 34.739 | 79.824 | 1.00 | 47.83 | C |
| ATOM | 12151 | CB | LYS | G | 14 | −9.084 | 34.934 | 79.231 | 1.00 | 47.78 | C |
| ATOM | 12154 | CG | LYS | G | 14 | −9.109 | 35.231 | 77.761 | 1.00 | 41.50 | C |
| ATOM | 12157 | CD | LYS | G | 14 | −10.542 | 35.229 | 77.263 | 1.00 | 47.17 | C |
| ATOM | 12160 | CE | LYS | G | 14 | −10.724 | 36.050 | 75.988 | 1.00 | 42.15 | C |
| ATOM | 12163 | NZ | LYS | G | 14 | −11.391 | 35.186 | 74.989 | 1.00 | 62.23 | N |
| ATOM | 12167 | C | LYS | G | 14 | −7.827 | 34.451 | 81.322 | 1.00 | 46.27 | C |
| ATOM | 12168 | O | LYS | G | 14 | −7.793 | 35.359 | 82.157 | 1.00 | 46.95 | O |
| ATOM | 12169 | N | VAL | G | 15 | −7.953 | 33.179 | 81.669 | 1.00 | 37.62 | N |
| ATOM | 12171 | CA | VAL | G | 15 | −8.050 | 32.845 | 83.077 | 1.00 | 42.86 | C |
| ATOM | 12173 | CB | VAL | G | 15 | −8.303 | 31.349 | 83.371 | 1.00 | 42.86 | C |
| ATOM | 12175 | CG1 | VAL | G | 15 | −8.045 | 31.046 | 84.855 | 1.00 | 50.94 | C |
| ATOM | 12179 | CG2 | VAL | G | 15 | −9.759 | 31.019 | 83.061 | 1.00 | 41.04 | C |
| ATOM | 12183 | C | VAL | G | 15 | −6.772 | 33.255 | 83.743 | 1.00 | 39.14 | C |
| ATOM | 12184 | O | VAL | G | 15 | −6.811 | 33.909 | 84.778 | 1.00 | 47.23 | O |
| ATOM | 12185 | N | LEU | G | 16 | −5.642 | 32.890 | 83.160 | 1.00 | 31.72 | N |
| ATOM | 12187 | CA | LEU | G | 16 | −4.382 | 33.156 | 83.851 | 1.00 | 40.34 | C |
| ATOM | 12189 | CB | LEU | G | 16 | −3.243 | 32.273 | 83.353 | 1.00 | 31.57 | C |
| ATOM | 12192 | CG | LEU | G | 16 | −3.175 | 31.031 | 84.241 | 1.00 | 47.71 | C |
| ATOM | 12194 | CD1 | LEU | G | 16 | −3.830 | 29.804 | 83.627 | 1.00 | 56.97 | C |
| ATOM | 12198 | CD2 | LEU | G | 16 | −1.767 | 30.699 | 84.684 | 1.00 | 57.71 | C |
| ATOM | 12202 | C | LEU | G | 16 | −3.997 | 34.640 | 83.877 | 1.00 | 41.31 | C |
| ATOM | 12203 | O | LEU | G | 16 | −3.357 | 35.111 | 84.798 | 1.00 | 43.90 | O |
| ATOM | 12204 | N | GLU | G | 17 | −4.491 | 35.418 | 82.928 | 1.00 | 44.13 | N |
| ATOM | 12206 | CA | GLU | G | 17 | −4.267 | 36.861 | 82.956 | 1.00 | 40.01 | C |
| ATOM | 12208 | CB | GLU | G | 17 | −4.781 | 37.465 | 81.641 | 1.00 | 43.81 | C |
| ATOM | 12211 | CG | GLU | G | 17 | −3.831 | 38.241 | 80.731 | 1.00 | 54.23 | C |
| ATOM | 12214 | CD | GLU | G | 17 | −2.384 | 37.779 | 80.728 | 1.00 | 70.24 | C |
| ATOM | 12215 | OE1 | GLU | G | 17 | −1.534 | 38.700 | 80.804 | 1.00 | 81.84 | O |
| ATOM | 12216 | OE2 | GLU | G | 17 | −2.102 | 36.551 | 80.628 | 1.00 | 77.75 | O |
| ATOM | 12217 | C | GLU | G | 17 | −5.103 | 37.434 | 84.078 | 1.00 | 41.94 | C |
| ATOM | 12218 | O | GLU | G | 17 | −4.981 | 38.590 | 84.444 | 1.00 | 44.94 | O |
| ATOM | 12219 | N | ALA | G | 18 | −6.098 | 36.683 | 84.513 | 1.00 | 45.11 | N |
| ATOM | 12221 | CA | ALA | G | 18 | −7.017 | 37.243 | 85.476 | 1.00 | 45.39 | C |
| ATOM | 12223 | CB | ALA | G | 18 | −8.372 | 36.724 | 85.213 | 1.00 | 43.48 | C |
| ATOM | 12227 | C | ALA | G | 18 | −6.554 | 36.851 | 86.877 | 1.00 | 47.42 | C |
| ATOM | 12228 | O | ALA | G | 18 | −7.109 | 37.303 | 87.866 | 1.00 | 51.43 | O |
| ATOM | 12229 | N | LEU | G | 19 | −5.533 | 36.007 | 86.977 | 1.00 | 46.49 | N |
| ATOM | 12231 | CA | LEU | G | 19 | −5.045 | 35.626 | 88.289 | 1.00 | 46.03 | C |
| ATOM | 12233 | CB | LEU | G | 19 | −5.033 | 34.114 | 88.473 | 1.00 | 52.00 | C |
| ATOM | 12236 | CG | LEU | G | 19 | −6.427 | 33.501 | 88.285 | 1.00 | 49.64 | C |
| ATOM | 12238 | CD1 | LEU | G | 19 | −6.412 | 31.981 | 88.416 | 1.00 | 67.56 | C |
| ATOM | 12242 | CD2 | LEU | G | 19 | −7.375 | 34.077 | 89.275 | 1.00 | 42.20 | C |
| ATOM | 12246 | C | LEU | G | 19 | −3.683 | 36.199 | 88.601 | 1.00 | 47.86 | C |
| ATOM | 12247 | O | LEU | G | 19 | −3.443 | 36.597 | 89.732 | 1.00 | 49.48 | O |
| ATOM | 12248 | N | LYS | G | 20 | −2.806 | 36.281 | 87.608 | 1.00 | 51.08 | N |
| ATOM | 12250 | CA | LYS | G | 20 | −1.370 | 36.327 | 87.878 | 1.00 | 48.86 | C |
| ATOM | 12252 | CB | LYS | G | 20 | −0.570 | 36.104 | 86.590 | 1.00 | 51.11 | C |
| ATOM | 12255 | CG | LYS | G | 20 | −0.600 | 37.240 | 85.569 | 1.00 | 48.72 | C |
| ATOM | 12258 | CD | LYS | G | 20 | 0.793 | 37.608 | 85.116 | 1.00 | 66.49 | C |
| ATOM | 12261 | CE | LYS | G | 20 | 0.784 | 38.858 | 84.273 | 1.00 | 65.43 | C |
| ATOM | 12264 | NZ | LYS | G | 20 | −0.275 | 38.797 | 83.258 | 1.00 | 58.00 | N |
| ATOM | 12268 | C | LYS | G | 20 | −0.968 | 37.615 | 88.599 | 1.00 | 44.03 | C |
| ATOM | 12269 | O | LYS | G | 20 | −0.128 | 37.595 | 89.498 | 1.00 | 42.42 | O |
| ATOM | 12270 | N | ASP | G | 21 | −1.658 | 38.709 | 88.302 | 1.00 | 46.16 | N |
| ATOM | 12272 | CA | ASP | G | 21 | −1.343 | 39.976 | 88.961 | 1.00 | 53.79 | C |
| ATOM | 12274 | CB | ASP | G | 21 | −1.923 | 41.140 | 88.161 | 1.00 | 57.04 | C |
| ATOM | 12277 | CG | ASP | G | 21 | −1.057 | 41.493 | 86.966 | 1.00 | 67.22 | C |
| ATOM | 12278 | OD1 | ASP | G | 21 | 0.174 | 41.330 | 87.102 | 1.00 | 65.04 | O |
| ATOM | 12279 | OD2 | ASP | G | 21 | −1.492 | 41.889 | 85.860 | 1.00 | 73.51 | O |
| ATOM | 12280 | C | ASP | G | 21 | −1.774 | 40.029 | 90.433 | 1.00 | 55.13 | C |
| ATOM | 12281 | O | ASP | G | 21 | −1.028 | 40.517 | 91.275 | 1.00 | 53.88 | O |
| ATOM | 12282 | N | LEU | G | 22 | −2.928 | 39.435 | 90.745 | 1.00 | 53.36 | N |
| ATOM | 12284 | CA | LEU | G | 22 | −3.415 | 39.260 | 92.120 | 1.00 | 47.12 | C |
| ATOM | 12286 | CB | LEU | G | 22 | −4.893 | 38.956 | 92.067 | 1.00 | 38.89 | C |
| ATOM | 12289 | CG | LEU | G | 22 | −5.524 | 38.953 | 93.447 | 1.00 | 44.78 | C |
| ATOM | 12291 | CD1 | LEU | G | 22 | −5.590 | 40.395 | 93.954 | 1.00 | 45.90 | C |
| ATOM | 12295 | CD2 | LEU | G | 22 | −6.929 | 38.288 | 93.417 | 1.00 | 53.45 | C |
| ATOM | 12299 | C | LEU | G | 22 | −2.794 | 38.184 | 93.031 | 1.00 | 45.78 | C |
| ATOM | 12300 | O | LEU | G | 22 | −2.593 | 38.391 | 94.228 | 1.00 | 47.85 | O |
| ATOM | 12301 | N | ILE | G | 23 | −2.607 | 36.988 | 92.506 | 1.00 | 40.24 | N |
| ATOM | 12303 | CA | ILE | G | 23 | −1.993 | 35.934 | 93.282 | 1.00 | 43.33 | C |
| ATOM | 12305 | CB | ILE | G | 23 | −2.991 | 34.771 | 93.653 | 1.00 | 48.42 | C |
| ATOM | 12307 | CG1 | ILE | G | 23 | −3.427 | 34.008 | 92.434 | 1.00 | 40.84 | C |
| ATOM | 12310 | CD1 | ILE | G | 23 | −4.816 | 33.488 | 92.596 | 1.00 | 64.22 | C |
| ATOM | 12314 | CG2 | ILE | G | 23 | −4.284 | 35.227 | 94.392 | 1.00 | 49.29 | C |
| ATOM | 12318 | C | ILE | G | 23 | −0.854 | 35.350 | 92.489 | 1.00 | 49.64 | C |
| ATOM | 12319 | O | ILE | G | 23 | −0.908 | 35.250 | 91.261 | 1.00 | 57.81 | O |
| ATOM | 12320 | N | ASN | G | 24 | 0.141 | 34.846 | 93.203 | 1.00 | 53.20 | N |
| ATOM | 12322 | CA | ASN | G | 24 | 1.410 | 34.491 | 92.573 | 1.00 | 54.30 | C |
| ATOM | 12324 | CB | ASN | G | 24 | 2.487 | 35.260 | 93.322 | 1.00 | 56.91 | C |
| ATOM | 12327 | CG | ASN | G | 24 | 1.893 | 36.060 | 94.482 | 1.00 | 80.52 | C |
| ATOM | 12328 | OD1 | ASN | G | 24 | 1.955 | 37.294 | 94.486 | 1.00 | 94.08 | O |
| ATOM | 12329 | ND2 | ASN | G | 24 | 1.244 | 35.363 | 95.437 | 1.00 | 90.02 | N |
| ATOM | 12332 | C | ASN | G | 24 | 1.647 | 32.977 | 92.611 | 1.00 | 46.95 | C |
| ATOM | 12333 | O | ASN | G | 24 | 2.197 | 32.392 | 91.683 | 1.00 | 52.56 | O |
| ATOM | 12334 | N | GLU | G | 25 | 1.194 | 32.330 | 93.672 | 1.00 | 44.21 | N |
| ATOM | 12336 | CA | GLU | G | 25 | 1.253 | 30.882 | 93.786 | 1.00 | 50.96 | C |
| ATOM | 12338 | CB | GLU | G | 25 | 2.249 | 30.492 | 94.894 | 1.00 | 55.58 | C |
| ATOM | 12341 | CG | GLU | G | 25 | 3.682 | 30.974 | 94.703 | 1.00 | 74.29 | C |
| ATOM | 12344 | CD | GLU | G | 25 | 4.372 | 31.407 | 95.999 | 1.00 | 100.88 | C |
| ATOM | 12345 | OE1 | GLU | G | 25 | 4.211 | 32.581 | 96.420 | 1.00 | 101.10 | O |
| ATOM | 12346 | OE2 | GLU | G | 25 | 5.123 | 30.585 | 96.582 | 1.00 | 109.04 | O |
| ATOM | 12347 | C | GLU | G | 25 | −0.155 | 30.408 | 94.168 | 1.00 | 49.23 | C |
| ATOM | 12348 | O | GLU | G | 25 | −0.829 | 31.040 | 94.985 | 1.00 | 48.64 | O |
| ATOM | 12349 | N | ALA | G | 26 | −0.603 | 29.293 | 93.595 | 1.00 | 49.27 | N |
| ATOM | 12351 | CA | ALA | G | 26 | −1.821 | 28.612 | 94.077 | 1.00 | 43.54 | C |
| ATOM | 12353 | CB | ALA | G | 26 | −3.055 | 29.272 | 93.584 | 1.00 | 33.03 | C |
| ATOM | 12357 | C | ALA | G | 26 | −1.886 | 27.136 | 93.723 | 1.00 | 48.55 | C |
| ATOM | 12358 | O | ALA | G | 26 | −1.109 | 26.588 | 92.914 | 1.00 | 48.20 | O |
| ATOM | 12359 | N | CYS | G | 27 | −2.802 | 26.476 | 94.409 | 1.00 | 50.64 | N |
| ATOM | 12361 | CA | CYS | G | 27 | −2.870 | 25.038 | 94.311 | 1.00 | 48.55 | C |
| ATOM | 12363 | CB | CYS | G | 27 | −3.091 | 24.404 | 95.666 | 1.00 | 44.22 | C |
| ATOM | 12366 | SG | CYS | G | 27 | −2.834 | 22.642 | 95.495 | 1.00 | 70.74 | S |
| ATOM | 12367 | C | CYS | G | 27 | −4.020 | 24.704 | 93.388 | 1.00 | 49.26 | C |
| ATOM | 12368 | O | CYS | G | 27 | −5.178 | 25.089 | 93.610 | 1.00 | 40.22 | O |
| ATOM | 12369 | N | TRP | G | 28 | −3.671 | 24.010 | 92.313 | 1.00 | 51.70 | N |
| ATOM | 12371 | CA | TRP | G | 28 | −4.650 | 23.650 | 91.294 | 1.00 | 47.07 | C |
| ATOM | 12373 | CB | TRP | G | 28 | −3.986 | 23.601 | 89.915 | 1.00 | 49.36 | C |
| ATOM | 12376 | CG | TRP | G | 28 | −3.749 | 24.968 | 89.335 | 1.00 | 49.27 | C |
| ATOM | 12377 | CD1 | TRP | G | 28 | −2.855 | 25.884 | 89.772 | 1.00 | 54.29 | C |
| ATOM | 12379 | NE1 | TRP | G | 28 | −2.921 | 27.018 | 89.000 | 1.00 | 54.92 | N |
| ATOM | 12381 | CE2 | TRP | G | 28 | −3.885 | 26.850 | 88.046 | 1.00 | 55.26 | C |
| ATOM | 12382 | CD2 | TRP | G | 28 | −4.424 | 25.574 | 88.221 | 1.00 | 52.67 | C |
| ATOM | 12383 | CE3 | TRP | G | 28 | −5.357 | 25.127 | 87.292 | 1.00 | 59.88 | C |
| ATOM | 12385 | CZ3 | TRP | G | 28 | −5.763 | 25.970 | 86.302 | 1.00 | 50.65 | C |
| ATOM | 12387 | CH2 | TRP | G | 28 | −5.248 | 27.241 | 86.183 | 1.00 | 65.05 | C |
| ATOM | 12389 | CZ2 | TRP | G | 28 | −4.279 | 27.687 | 87.022 | 1.00 | 69.46 | C |
| ATOM | 12391 | C | TRP | G | 28 | −5.166 | 22.293 | 91.691 | 1.00 | 42.58 | C |
| ATOM | 12392 | O | TRP | G | 28 | −4.476 | 21.286 | 91.559 | 1.00 | 46.97 | O |
| ATOM | 12393 | N | ASP | G | 29 | −6.355 | 22.275 | 92.270 | 1.00 | 48.34 | N |
| ATOM | 12395 | CA | ASP | G | 29 | −6.966 | 21.015 | 92.699 | 1.00 | 48.63 | C |
| ATOM | 12397 | CB | ASP | G | 29 | −8.021 | 21.320 | 93.747 | 1.00 | 43.99 | C |
| ATOM | 12400 | CG | ASP | G | 29 | −7.406 | 21.745 | 95.038 | 1.00 | 56.01 | C |
| ATOM | 12401 | OD1 | ASP | G | 29 | −6.600 | 20.929 | 95.568 | 1.00 | 63.17 | O |
| ATOM | 12402 | OD2 | ASP | G | 29 | −7.621 | 22.884 | 95.525 | 1.00 | 60.99 | O |
| ATOM | 12403 | C | ASP | G | 29 | −7.603 | 20.271 | 91.532 | 1.00 | 48.42 | C |
| ATOM | 12404 | O | ASP | G | 29 | −8.626 | 20.717 | 91.000 | 1.00 | 45.88 | O |
| ATOM | 12405 | N | ILE | G | 30 | −6.988 | 19.159 | 91.132 | 1.00 | 47.05 | N |
| ATOM | 12407 | CA | ILE | G | 30 | −7.486 | 18.386 | 90.003 | 1.00 | 50.90 | C |
| ATOM | 12409 | CB | ILE | G | 30 | −6.328 | 18.192 | 89.031 | 1.00 | 54.62 | C |
| ATOM | 12411 | CG1 | ILE | G | 30 | −5.874 | 19.588 | 88.569 | 1.00 | 59.11 | C |
| ATOM | 12414 | CD1 | ILE | G | 30 | −5.704 | 19.705 | 87.068 | 1.00 | 72.47 | C |
| ATOM | 12418 | CG2 | ILE | G | 30 | −6.716 | 17.290 | 87.839 | 1.00 | 52.18 | C |
| ATOM | 12422 | C | ILE | G | 30 | −8.167 | 17.079 | 90.399 | 1.00 | 52.73 | C |
| ATOM | 12423 | O | ILE | G | 30 | −7.673 | 16.367 | 91.265 | 1.00 | 66.15 | O |
| ATOM | 12424 | N | SER | G | 31 | −9.354 | 16.830 | 89.845 | 1.00 | 59.55 | N |
| ATOM | 12426 | CA | SER | G | 31 | −10.167 | 15.625 | 90.117 | 1.00 | 56.78 | C |
| ATOM | 12428 | CB | SER | G | 31 | −11.096 | 15.829 | 91.322 | 1.00 | 56.10 | C |
| ATOM | 12431 | OG | SER | G | 31 | −12.311 | 16.495 | 90.960 | 1.00 | 37.98 | O |
| ATOM | 12433 | C | SER | G | 31 | −11.072 | 15.292 | 88.928 | 1.00 | 58.56 | C |
| ATOM | 12434 | O | SER | G | 31 | −11.282 | 16.114 | 88.039 | 1.00 | 59.80 | O |
| ATOM | 12435 | N | SER | G | 32 | −11.719 | 14.138 | 89.008 | 1.00 | 63.76 | N |
| ATOM | 12437 | CA | SER | G | 32 | −12.632 | 13.673 | 87.974 | 1.00 | 64.54 | C |
| ATOM | 12439 | CB | SER | G | 32 | −13.245 | 12.354 | 88.401 | 1.00 | 58.58 | C |
| ATOM | 12442 | OG | SER | G | 32 | −12.405 | 11.358 | 87.858 | 1.00 | 75.65 | O |
| ATOM | 12444 | C | SER | G | 32 | −13.744 | 14.635 | 87.597 | 1.00 | 60.96 | C |
| ATOM | 12445 | O | SER | G | 32 | −14.248 | 14.616 | 86.474 | 1.00 | 68.45 | O |
| ATOM | 12446 | N | SER | G | 33 | −14.140 | 15.492 | 88.519 | 1.00 | 55.34 | N |
| ATOM | 12448 | CA | SER | G | 33 | −15.242 | 16.374 | 88.202 | 1.00 | 52.95 | C |
| ATOM | 12450 | CB | SER | G | 33 | −16.083 | 16.638 | 89.458 | 1.00 | 55.53 | C |
| ATOM | 12453 | OG | SER | G | 33 | −15.561 | 17.731 | 90.186 | 1.00 | 56.98 | O |
| ATOM | 12455 | C | SER | G | 33 | −14.691 | 17.663 | 87.607 | 1.00 | 50.38 | C |
| ATOM | 12456 | O | SER | G | 33 | −15.439 | 18.481 | 87.049 | 1.00 | 47.79 | O |
| ATOM | 12457 | N | GLY | G | 34 | −13.380 | 17.848 | 87.760 | 1.00 | 51.31 | N |
| ATOM | 12459 | CA | GLY | G | 34 | −12.670 | 18.874 | 87.012 | 1.00 | 52.70 | C |
| ATOM | 12462 | C | GLY | G | 34 | −11.621 | 19.693 | 87.739 | 1.00 | 49.57 | C |
| ATOM | 12463 | O | GLY | G | 34 | −10.908 | 19.198 | 88.613 | 1.00 | 46.99 | O |
| ATOM | 12464 | N | VAL | G | 35 | −11.495 | 20.948 | 87.326 | 1.00 | 44.86 | N |
| ATOM | 12466 | CA | VAL | G | 35 | −10.475 | 21.826 | 87.880 | 1.00 | 43.78 | C |
| ATOM | 12468 | CB | VAL | G | 35 | −9.780 | 22.612 | 86.770 | 1.00 | 43.39 | C |
| ATOM | 12470 | CG1 | VAL | G | 35 | −8.787 | 23.608 | 87.365 | 1.00 | 43.80 | C |
| ATOM | 12474 | CG2 | VAL | G | 35 | −9.095 | 21.674 | 85.780 | 1.00 | 44.68 | C |
| ATOM | 12478 | C | VAL | G | 35 | −10.965 | 22.828 | 88.939 | 1.00 | 49.70 | C |
| ATOM | 12479 | O | VAL | G | 35 | −11.896 | 23.636 | 88.714 | 1.00 | 45.52 | O |
| ATOM | 12480 | N | ASN | G | 36 | −10.273 | 22.817 | 90.078 | 1.00 | 49.96 | N |
| ATOM | 12482 | CA | ASN | G | 36 | −10.612 | 23.726 | 91.168 | 1.00 | 55.67 | C |
| ATOM | 12484 | CB | ASN | G | 36 | −11.399 | 22.977 | 92.236 | 1.00 | 57.04 | C |
| ATOM | 12487 | CG | ASN | G | 36 | −12.857 | 22.818 | 91.859 | 1.00 | 62.63 | C |
| ATOM | 12488 | OD1 | ASN | G | 36 | −13.670 | 23.737 | 92.068 | 1.00 | 47.94 | O |
| ATOM | 12489 | ND2 | ASN | G | 36 | −13.183 | 21.677 | 91.241 | 1.00 | 67.31 | N |
| ATOM | 12492 | C | ASN | G | 36 | −9.457 | 24.489 | 91.810 | 1.00 | 52.54 | C |
| ATOM | 12493 | O | ASN | G | 36 | −8.382 | 23.939 | 92.028 | 1.00 | 58.19 | O |
| ATOM | 12494 | N | LEU | G | 37 | −9.688 | 25.767 | 92.102 | 1.00 | 44.49 | N |
| ATOM | 12496 | CA | LEU | G | 37 | −8.633 | 26.629 | 92.640 | 1.00 | 42.56 | C |
| ATOM | 12498 | CB | LEU | G | 37 | −7.912 | 27.347 | 91.521 | 1.00 | 39.15 | C |
| ATOM | 12501 | CG | LEU | G | 37 | −6.704 | 28.188 | 91.901 | 1.00 | 41.35 | C |
| ATOM | 12503 | CD1 | LEU | G | 37 | −5.635 | 28.067 | 90.791 | 1.00 | 50.86 | C |
| ATOM | 12507 | CD2 | LEU | G | 37 | −7.181 | 29.629 | 92.016 | 1.00 | 45.53 | C |
| ATOM | 12511 | C | LEU | G | 37 | −9.135 | 27.709 | 93.595 | 1.00 | 40.17 | C |
| ATOM | 12512 | O | LEU | G | 37 | −9.979 | 28.527 | 93.213 | 1.00 | 36.77 | O |
| ATOM | 12513 | N | GLN | G | 38 | −8.565 | 27.737 | 94.799 | 1.00 | 32.29 | N |
| ATOM | 12515 | CA | GLN | G | 38 | −8.897 | 28.764 | 95.784 | 1.00 | 37.74 | C |
| ATOM | 12517 | CB | GLN | G | 38 | −9.780 | 28.115 | 96.828 | 1.00 | 40.10 | C |
| ATOM | 12520 | CG | GLN | G | 38 | −10.226 | 29.019 | 97.938 | 1.00 | 55.60 | C |
| ATOM | 12523 | CD | GLN | G | 38 | −10.482 | 28.224 | 99.191 | 1.00 | 39.91 | C |
| ATOM | 12524 | OE1 | GLN | G | 38 | −9.569 | 27.923 | 99.968 | 1.00 | 56.28 | O |
| ATOM | 12525 | NE2 | GLN | G | 38 | −11.717 | 27.837 | 99.357 | 1.00 | 47.11 | N |
| ATOM | 12528 | C | GLN | G | 38 | −7.630 | 29.337 | 96.444 | 1.00 | 39.91 | C |
| ATOM | 12529 | O | GLN | G | 38 | −6.816 | 28.573 | 96.957 | 1.00 | 35.19 | O |
| ATOM | 12530 | N | SER | G | 39 | −7.376 | 30.635 | 96.288 | 1.00 | 37.76 | N |
| ATOM | 12532 | CA | SER | G | 39 | −6.250 | 31.253 | 96.972 | 1.00 | 46.67 | C |
| ATOM | 12534 | CB | SER | G | 39 | −4.909 | 31.215 | 96.228 | 1.00 | 47.58 | C |
| ATOM | 12537 | OG | SER | G | 39 | −3.941 | 30.485 | 97.008 | 1.00 | 60.44 | O |
| ATOM | 12539 | C | SER | G | 39 | −6.590 | 32.654 | 97.348 | 1.00 | 50.80 | C |
| ATOM | 12540 | O | SER | G | 39 | −7.521 | 33.277 | 96.792 | 1.00 | 50.16 | O |
| ATOM | 12541 | N | MET | G | 40 | −5.877 | 33.090 | 98.382 | 1.00 | 50.07 | N |
| ATOM | 12543 | CA | MET | G | 40 | −5.971 | 34.471 | 98.818 | 1.00 | 51.70 | C |
| ATOM | 12545 | CB | MET | G | 40 | −5.991 | 34.540 | 100.335 | 1.00 | 48.33 | C |
| ATOM | 12548 | CG | MET | G | 40 | −7.023 | 33.651 | 100.985 | 1.00 | 52.47 | C |
| ATOM | 12551 | SD | MET | G | 40 | −7.204 | 34.045 | 102.745 | 1.00 | 45.35 | S |
| ATOM | 12552 | CE | MET | G | 40 | −6.054 | 32.929 | 103.414 | 1.00 | 30.99 | C |
| ATOM | 12556 | C | MET | G | 40 | −4.778 | 35.244 | 98.272 | 1.00 | 51.75 | C |
| ATOM | 12557 | O | MET | G | 40 | −3.765 | 34.679 | 97.896 | 1.00 | 56.52 | O |
| ATOM | 12558 | N | ASP | G | 41 | −4.882 | 36.557 | 98.240 | 1.00 | 51.83 | N |
| ATOM | 12560 | CA | ASP | G | 41 | −3.698 | 37.345 | 97.982 | 1.00 | 56.49 | C |
| ATOM | 12562 | CB | ASP | G | 41 | −4.131 | 38.706 | 97.430 | 1.00 | 61.92 | C |
| ATOM | 12565 | CG | ASP | G | 41 | −4.385 | 39.711 | 98.500 | 1.00 | 51.46 | C |
| ATOM | 12566 | OD1 | ASP | G | 41 | −5.431 | 39.584 | 99.132 | 1.00 | 48.98 | O |
| ATOM | 12567 | OD2 | ASP | G | 41 | −3.627 | 40.672 | 98.735 | 1.00 | 70.79 | O |
| ATOM | 12568 | C | ASP | G | 41 | −2.724 | 37.409 | 99.183 | 1.00 | 52.99 | C |
| ATOM | 12569 | O | ASP | G | 41 | −2.980 | 36.813 | 100.230 | 1.00 | 50.30 | O |
| ATOM | 12570 | N | SER | G | 42 | −1.546 | 37.983 | 98.964 | 1.00 | 45.42 | N |
| ATOM | 12572 | CA | SER | G | 42 | −0.540 | 38.115 | 100.004 | 1.00 | 50.65 | C |
| ATOM | 12574 | CB | SER | G | 42 | 0.564 | 39.064 | 99.515 | 1.00 | 56.16 | C |
| ATOM | 12577 | OG | SER | G | 42 | 1.420 | 38.449 | 98.587 | 1.00 | 65.05 | O |
| ATOM | 12579 | C | SER | G | 42 | −1.131 | 38.716 | 101.301 | 1.00 | 48.35 | C |
| ATOM | 12580 | O | SER | G | 42 | −0.673 | 38.385 | 102.402 | 1.00 | 38.02 | O |
| ATOM | 12581 | N | SER | G | 43 | −2.083 | 39.641 | 101.173 | 1.00 | 39.34 | N |
| ATOM | 12583 | CA | SER | G | 43 | −2.466 | 40.488 | 102.286 | 1.00 | 41.69 | C |
| ATOM | 12585 | CB | SER | G | 43 | −2.734 | 41.897 | 101.770 | 1.00 | 47.07 | C |
| ATOM | 12588 | OG | SER | G | 43 | −3.891 | 41.925 | 100.953 | 1.00 | 46.21 | O |
| ATOM | 12590 | C | SER | G | 43 | −3.717 | 39.914 | 102.938 | 1.00 | 47.03 | C |
| ATOM | 12591 | O | SER | G | 43 | −4.287 | 40.492 | 103.864 | 1.00 | 50.12 | O |
| ATOM | 12592 | N | HIS | G | 44 | −4.153 | 38.783 | 102.391 | 1.00 | 53.62 | N |
| ATOM | 12594 | CA | HIS | G | 44 | −5.234 | 37.978 | 102.940 | 1.00 | 46.98 | C |
| ATOM | 12596 | CB | HIS | G | 44 | −4.855 | 37.392 | 104.290 | 1.00 | 42.27 | C |
| ATOM | 12599 | CG | HIS | G | 44 | −4.024 | 36.154 | 104.179 | 1.00 | 42.21 | C |
| ATOM | 12600 | ND1 | HIS | G | 44 | −4.043 | 35.154 | 105.124 | 1.00 | 57.65 | N |
| ATOM | 12602 | CE1 | HIS | G | 44 | −3.171 | 34.217 | 104.796 | 1.00 | 66.80 | C |
| ATOM | 12604 | NE2 | HIS | G | 44 | −2.597 | 34.562 | 103.659 | 1.00 | 65.56 | N |
| ATOM | 12606 | CD2 | HIS | G | 44 | −3.110 | 35.773 | 103.257 | 1.00 | 62.64 | C |
| ATOM | 12608 | C | HIS | G | 44 | −6.503 | 38.789 | 103.047 | 1.00 | 42.62 | C |
| ATOM | 12609 | O | HIS | G | 44 | −7.308 | 38.585 | 103.927 | 1.00 | 40.82 | O |
| ATOM | 12610 | N | VAL | G | 45 | −6.676 | 39.708 | 102.122 | 1.00 | 40.51 | N |
| ATOM | 12612 | CA | VAL | G | 45 | −7.826 | 40.591 | 102.142 | 1.00 | 48.67 | C |
| ATOM | 12614 | CB | VAL | G | 45 | −7.349 | 41.998 | 101.788 | 1.00 | 47.95 | C |
| ATOM | 12616 | CG1 | VAL | G | 45 | −8.471 | 42.836 | 101.282 | 1.00 | 44.00 | C |
| ATOM | 12620 | CG2 | VAL | G | 45 | −6.612 | 42.623 | 102.935 | 1.00 | 51.56 | C |
| ATOM | 12624 | C | VAL | G | 45 | −8.824 | 40.235 | 101.047 | 1.00 | 51.64 | C |
| ATOM | 12625 | O | VAL | G | 45 | −9.914 | 40.778 | 101.034 | 1.00 | 54.27 | O |
| ATOM | 12626 | N | SER | G | 46 | −8.376 | 39.470 | 100.052 | 1.00 | 54.87 | N |
| ATOM | 12628 | CA | SER | G | 46 | −9.217 | 38.922 | 98.992 | 1.00 | 48.19 | C |
| ATOM | 12630 | CB | SER | G | 46 | −9.005 | 39.626 | 97.649 | 1.00 | 51.48 | C |
| ATOM | 12633 | OG | SER | G | 46 | −7.704 | 39.350 | 97.139 | 1.00 | 54.66 | O |
| ATOM | 12635 | C | SER | G | 46 | −8.904 | 37.459 | 98.790 | 1.00 | 48.86 | C |
| ATOM | 12636 | O | SER | G | 46 | −7.770 | 36.992 | 98.882 | 1.00 | 44.70 | O |
| ATOM | 12637 | N | LEU | G | 47 | −9.945 | 36.741 | 98.415 | 1.00 | 50.61 | N |
| ATOM | 12639 | CA | LEU | G | 47 | −9.782 | 35.368 | 98.006 | 1.00 | 46.23 | C |
| ATOM | 12641 | CB | LEU | G | 47 | −10.618 | 34.539 | 98.961 | 1.00 | 48.37 | C |
| ATOM | 12644 | CG | LEU | G | 47 | −10.761 | 33.092 | 98.546 | 1.00 | 50.44 | C |
| ATOM | 12646 | CD1 | LEU | G | 47 | −10.575 | 32.228 | 99.755 | 1.00 | 61.17 | C |
| ATOM | 12650 | CD2 | LEU | G | 47 | −12.125 | 32.963 | 97.979 | 1.00 | 61.19 | C |
| ATOM | 12654 | C | LEU | G | 47 | −10.321 | 35.234 | 96.593 | 1.00 | 43.83 | C |
| ATOM | 12655 | O | LEU | G | 47 | −11.196 | 36.003 | 96.145 | 1.00 | 38.36 | O |
| ATOM | 12656 | N | VAL | G | 48 | −9.808 | 34.250 | 95.876 | 1.00 | 33.52 | N |
| ATOM | 12658 | CA | VAL | G | 48 | −10.370 | 34.009 | 94.562 | 1.00 | 37.65 | C |
| ATOM | 12660 | CB | VAL | G | 48 | −9.395 | 34.399 | 93.394 | 1.00 | 44.44 | C |
| ATOM | 12662 | CG1 | VAL | G | 48 | −8.158 | 33.531 | 93.386 | 1.00 | 39.78 | C |
| ATOM | 12666 | CG2 | VAL | G | 48 | −10.058 | 34.160 | 92.043 | 1.00 | 62.26 | C |
| ATOM | 12670 | C | VAL | G | 48 | −10.656 | 32.540 | 94.457 | 1.00 | 31.91 | C |
| ATOM | 12671 | O | VAL | G | 48 | −9.844 | 31.728 | 94.869 | 1.00 | 29.91 | O |
| ATOM | 12672 | N | GLN | G | 49 | −11.756 | 32.194 | 93.804 | 1.00 | 36.79 | N |
| ATOM | 12674 | CA | GLN | G | 49 | −11.970 | 30.801 | 93.455 | 1.00 | 42.45 | C |
| ATOM | 12676 | CB | GLN | G | 49 | −12.976 | 30.172 | 94.427 | 1.00 | 49.76 | C |
| ATOM | 12679 | CG | GLN | G | 49 | −13.708 | 28.907 | 93.984 | 1.00 | 67.82 | C |
| ATOM | 12682 | CD | GLN | G | 49 | −15.202 | 28.962 | 94.345 | 1.00 | 90.44 | C |
| ATOM | 12683 | OE1 | GLN | G | 49 | −15.573 | 28.956 | 95.531 | 1.00 | 91.03 | O |
| ATOM | 12684 | NE2 | GLN | G | 49 | −16.055 | 29.044 | 93.319 | 1.00 | 82.85 | N |
| ATOM | 12687 | C | GLN | G | 49 | −12.350 | 30.590 | 91.997 | 1.00 | 41.83 | C |
| ATOM | 12688 | O | GLN | G | 49 | −13.159 | 31.318 | 91.391 | 1.00 | 42.00 | O |
| ATOM | 12689 | N | LEU | G | 50 | −11.761 | 29.535 | 91.457 | 1.00 | 36.33 | N |
| ATOM | 12691 | CA | LEU | G | 50 | −11.922 | 29.200 | 90.051 | 1.00 | 36.39 | C |
| ATOM | 12693 | CB | LEU | G | 50 | −10.582 | 29.287 | 89.331 | 1.00 | 31.49 | C |
| ATOM | 12696 | CG | LEU | G | 50 | −10.560 | 28.660 | 87.957 | 1.00 | 33.12 | C |
| ATOM | 12698 | CD1 | LEU | G | 50 | −10.992 | 29.666 | 86.903 | 1.00 | 41.24 | C |
| ATOM | 12702 | CD2 | LEU | G | 50 | −9.198 | 28.081 | 87.679 | 1.00 | 37.61 | C |
| ATOM | 12706 | C | LEU | G | 50 | −12.415 | 27.771 | 89.978 | 1.00 | 39.12 | C |
| ATOM | 12707 | O | LEU | G | 50 | −11.985 | 26.886 | 90.750 | 1.00 | 42.70 | O |
| ATOM | 12708 | N | THR | G | 51 | −13.334 | 27.581 | 89.043 | 1.00 | 37.44 | N |
| ATOM | 12710 | CA | THR | G | 51 | −14.015 | 26.315 | 88.842 | 1.00 | 42.88 | C |
| ATOM | 12712 | CB | THR | G | 51 | −15.415 | 26.366 | 89.483 | 1.00 | 48.80 | C |
| ATOM | 12714 | OG1 | THR | G | 51 | −15.278 | 26.597 | 90.890 | 1.00 | 65.02 | O |
| ATOM | 12716 | CG2 | THR | G | 51 | −16.130 | 25.000 | 89.379 | 1.00 | 61.86 | C |
| ATOM | 12720 | C | THR | G | 51 | −14.203 | 26.133 | 87.359 | 1.00 | 43.99 | C |
| ATOM | 12721 | O | THR | G | 51 | −14.871 | 26.948 | 86.702 | 1.00 | 35.30 | O |
| ATOM | 12722 | N | LEU | G | 52 | −13.625 | 25.045 | 86.862 | 1.00 | 45.58 | N |
| ATOM | 12724 | CA | LEU | G | 52 | −13.716 | 24.680 | 85.451 | 1.00 | 47.25 | C |
| ATOM | 12726 | CB | LEU | G | 52 | −12.340 | 24.815 | 84.755 | 1.00 | 48.27 | C |
| ATOM | 12729 | CG | LEU | G | 52 | −11.798 | 26.245 | 84.646 | 1.00 | 53.80 | C |
| ATOM | 12731 | CD1 | LEU | G | 52 | −10.365 | 26.244 | 84.116 | 1.00 | 50.70 | C |
| ATOM | 12735 | CD2 | LEU | G | 52 | −12.708 | 27.099 | 83.741 | 1.00 | 44.13 | C |
| ATOM | 12739 | C | LEU | G | 52 | −14.146 | 23.226 | 85.445 | 1.00 | 41.96 | C |
| ATOM | 12740 | O | LEU | G | 52 | −13.464 | 22.372 | 86.015 | 1.00 | 39.88 | O |
| ATOM | 12741 | N | ARG | G | 53 | −15.284 | 22.940 | 84.825 | 1.00 | 47.16 | N |
| ATOM | 12743 | CA | ARG | G | 53 | −15.907 | 21.605 | 84.923 | 1.00 | 44.65 | C |
| ATOM | 12745 | CB | ARG | G | 53 | −17.402 | 21.698 | 84.623 | 1.00 | 43.37 | C |
| ATOM | 12748 | CG | ARG | G | 53 | −18.289 | 22.112 | 85.789 | 1.00 | 37.49 | C |
| ATOM | 12751 | CD | ARG | G | 53 | −19.757 | 22.341 | 85.399 | 1.00 | 43.12 | C |
| ATOM | 12754 | NE | ARG | G | 53 | −20.082 | 23.760 | 85.333 | 1.00 | 49.18 | N |
| ATOM | 12756 | CZ | ARG | G | 53 | −20.368 | 24.415 | 84.224 | 1.00 | 65.63 | C |
| ATOM | 12757 | NH1 | ARG | G | 53 | −20.354 | 23.801 | 83.047 | 1.00 | 77.75 | N |
| ATOM | 12760 | NH2 | ARG | G | 53 | −20.670 | 25.702 | 84.301 | 1.00 | 82.74 | N |
| ATOM | 12763 | C | ARG | G | 53 | −15.320 | 20.731 | 83.849 | 1.00 | 37.14 | C |
| ATOM | 12764 | O | ARG | G | 53 | −15.122 | 21.194 | 82.746 | 1.00 | 45.80 | O |
| ATOM | 12765 | N | SER | G | 54 | −15.137 | 19.455 | 84.118 | 1.00 | 37.57 | N |
| ATOM | 12767 | CA | SER | G | 54 | −14.536 | 18.570 | 83.124 | 1.00 | 41.47 | C |
| ATOM | 12769 | CB | SER | G | 54 | −14.444 | 17.133 | 83.651 | 1.00 | 48.26 | C |
| ATOM | 12772 | OG | SER | G | 54 | −15.721 | 16.592 | 83.954 | 1.00 | 46.17 | O |
| ATOM | 12774 | C | SER | G | 54 | −15.320 | 18.541 | 81.838 | 1.00 | 41.89 | C |
| ATOM | 12775 | O | SER | G | 54 | −14.721 | 18.453 | 80.799 | 1.00 | 43.16 | O |
| ATOM | 12776 | N | GLU | G | 55 | −16.648 | 18.559 | 81.907 | 1.00 | 46.68 | N |
| ATOM | 12778 | CA | GLU | G | 55 | −17.483 | 18.699 | 80.711 | 1.00 | 47.00 | C |
| ATOM | 12780 | CB | GLU | G | 55 | −18.871 | 19.207 | 81.087 | 1.00 | 47.62 | C |
| ATOM | 12783 | CG | GLU | G | 55 | −19.640 | 18.220 | 81.919 | 1.00 | 53.97 | C |
| ATOM | 12786 | CD | GLU | G | 55 | −19.469 | 18.483 | 83.388 | 1.00 | 64.90 | C |
| ATOM | 12787 | OE1 | GLU | G | 55 | −20.347 | 19.200 | 83.953 | 1.00 | 63.13 | O |
| ATOM | 12788 | OE2 | GLU | G | 55 | −18.458 | 17.957 | 83.920 | 1.00 | 53.33 | O |
| ATOM | 12789 | C | GLU | G | 55 | −16.927 | 19.723 | 79.760 | 1.00 | 42.31 | C |
| ATOM | 12790 | O | GLU | G | 55 | −17.096 | 19.631 | 78.548 | 1.00 | 52.71 | O |
| ATOM | 12791 | N | GLY | G | 56 | −16.405 | 20.787 | 80.342 | 1.00 | 37.72 | N |
| ATOM | 12793 | CA | GLY | G | 56 | −16.085 | 21.983 | 79.586 | 1.00 | 42.09 | C |
| ATOM | 12796 | C | GLY | G | 56 | −14.899 | 21.765 | 78.679 | 1.00 | 37.21 | C |
| ATOM | 12797 | O | GLY | G | 56 | −14.673 | 22.528 | 77.762 | 1.00 | 37.03 | O |
| ATOM | 12798 | N | PHE | G | 57 | −14.159 | 20.700 | 78.943 | 1.00 | 37.68 | N |
| ATOM | 12800 | CA | PHE | G | 57 | −13.084 | 20.264 | 78.080 | 1.00 | 43.27 | C |
| ATOM | 12802 | CB | PHE | G | 57 | −11.974 | 19.675 | 78.949 | 1.00 | 41.60 | C |
| ATOM | 12805 | CG | PHE | G | 57 | −11.334 | 20.656 | 79.918 | 1.00 | 41.77 | C |
| ATOM | 12806 | CD1 | PHE | G | 57 | −11.805 | 20.802 | 81.211 | 1.00 | 48.06 | C |
| ATOM | 12808 | CE1 | PHE | G | 57 | −11.160 | 21.628 | 82.122 | 1.00 | 39.54 | C |
| ATOM | 12810 | CZ | PHE | G | 57 | −9.990 | 22.268 | 81.764 | 1.00 | 41.02 | C |
| ATOM | 12812 | CE2 | PHE | G | 57 | −9.513 | 22.146 | 80.485 | 1.00 | 36.18 | C |
| ATOM | 12814 | CD2 | PHE | G | 57 | −10.164 | 21.315 | 79.579 | 1.00 | 52.45 | C |
| ATOM | 12816 | C | PHE | G | 57 | −13.574 | 19.253 | 77.027 | 1.00 | 46.17 | C |
| ATOM | 12817 | O | PHE | G | 57 | −14.620 | 18.642 | 77.198 | 1.00 | 65.68 | O |
| ATOM | 12818 | N | ASP | G | 58 | −12.854 | 19.080 | 75.926 | 1.00 | 47.38 | N |
| ATOM | 12820 | CA | ASP | G | 58 | −13.172 | 18.016 | 74.982 | 1.00 | 51.88 | C |
| ATOM | 12822 | CB | ASP | G | 58 | −12.552 | 18.280 | 73.621 | 1.00 | 56.06 | C |
| ATOM | 12825 | CG | ASP | G | 58 | −13.300 | 19.341 | 72.854 | 1.00 | 75.54 | C |
| ATOM | 12826 | OD1 | ASP | G | 58 | −13.865 | 18.979 | 71.796 | 1.00 | 98.98 | O |
| ATOM | 12827 | OD2 | ASP | G | 58 | −13.404 | 20.530 | 73.249 | 1.00 | 84.50 | O |
| ATOM | 12828 | C | ASP | G | 58 | −12.675 | 16.664 | 75.440 | 1.00 | 59.65 | C |
| ATOM | 12829 | O | ASP | G | 58 | −13.133 | 15.630 | 74.995 | 1.00 | 68.27 | O |
| ATOM | 12830 | N | THR | G | 59 | −11.749 | 16.636 | 76.376 | 1.00 | 65.80 | N |
| ATOM | 12832 | CA | THR | G | 59 | −11.108 | 15.375 | 76.692 | 1.00 | 56.12 | C |
| ATOM | 12834 | CB | THR | G | 59 | −9.871 | 15.299 | 75.856 | 1.00 | 57.63 | C |
| ATOM | 12836 | OG1 | THR | G | 59 | −10.237 | 14.706 | 74.619 | 1.00 | 49.73 | O |
| ATOM | 12838 | CG2 | THR | G | 59 | −8.760 | 14.457 | 76.506 | 1.00 | 58.31 | C |
| ATOM | 12842 | C | THR | G | 59 | −10.711 | 15.588 | 78.100 | 1.00 | 49.87 | C |
| ATOM | 12843 | O | THR | G | 59 | −10.194 | 16.647 | 78.394 | 1.00 | 53.68 | O |
| ATOM | 12844 | N | TYR | G | 60 | −10.963 | 14.629 | 78.974 | 1.00 | 51.83 | N |
| ATOM | 12846 | CA | TYR | G | 60 | −10.610 | 14.820 | 80.387 | 1.00 | 51.57 | C |
| ATOM | 12848 | CB | TYR | G | 60 | −11.707 | 15.541 | 81.153 | 1.00 | 37.85 | C |
| ATOM | 12851 | CG | TYR | G | 60 | −11.261 | 16.167 | 82.431 | 1.00 | 56.28 | C |
| ATOM | 12852 | CD1 | TYR | G | 60 | −11.009 | 17.532 | 82.515 | 1.00 | 58.58 | C |
| ATOM | 12854 | CE1 | TYR | G | 60 | −10.646 | 18.120 | 83.724 | 1.00 | 45.60 | C |
| ATOM | 12856 | CZ | TYR | G | 60 | −10.548 | 17.349 | 84.849 | 1.00 | 41.53 | C |
| ATOM | 12857 | OH | TYR | G | 60 | −10.153 | 17.889 | 86.047 | 1.00 | 54.36 | O |
| ATOM | 12859 | CE2 | TYR | G | 60 | −10.844 | 16.011 | 84.800 | 1.00 | 64.07 | C |
| ATOM | 12861 | CD2 | TYR | G | 60 | −11.196 | 15.423 | 83.597 | 1.00 | 68.92 | C |
| ATOM | 12863 | C | TYR | G | 60 | −10.459 | 13.462 | 80.974 | 1.00 | 49.22 | C |
| ATOM | 12864 | O | TYR | G | 60 | −11.359 | 12.661 | 80.863 | 1.00 | 55.61 | O |
| ATOM | 12865 | N | ARG | G | 61 | −9.317 | 13.202 | 81.581 | 1.00 | 55.92 | N |
| ATOM | 12867 | CA | ARG | G | 61 | −9.068 | 11.914 | 82.192 | 1.00 | 57.67 | C |
| ATOM | 12869 | CB | ARG | G | 61 | −8.086 | 11.107 | 81.328 | 1.00 | 64.20 | C |
| ATOM | 12872 | CG | ARG | G | 61 | −7.692 | 9.717 | 81.834 | 1.00 | 73.40 | C |
| ATOM | 12875 | CD | ARG | G | 61 | −7.346 | 8.713 | 80.735 | 1.00 | 86.87 | C |
| ATOM | 12878 | NE | ARG | G | 61 | −6.057 | 8.060 | 80.984 | 1.00 | 93.82 | N |
| ATOM | 12880 | CZ | ARG | G | 61 | −4.999 | 8.151 | 80.178 | 1.00 | 92.32 | C |
| ATOM | 12881 | NH1 | ARG | G | 61 | −5.061 | 8.877 | 79.064 | 1.00 | 94.52 | N |
| ATOM | 12884 | NH2 | ARG | G | 61 | −3.875 | 7.512 | 80.480 | 1.00 | 80.52 | N |
| ATOM | 12887 | C | ARG | G | 61 | −8.406 | 12.316 | 83.480 | 1.00 | 62.13 | C |
| ATOM | 12888 | O | ARG | G | 61 | −7.409 | 13.040 | 83.489 | 1.00 | 61.44 | O |
| ATOM | 12889 | N | CYS | G | 62 | −8.905 | 11.789 | 84.577 | 1.00 | 66.22 | N |
| ATOM | 12891 | CA | CYS | G | 62 | −8.229 | 12.066 | 85.827 | 1.00 | 70.89 | C |
| ATOM | 12893 | CB | CYS | G | 62 | −8.968 | 13.212 | 86.536 | 1.00 | 75.52 | C |
| ATOM | 12896 | SG | CYS | G | 62 | −8.142 | 13.784 | 88.033 | 1.00 | 73.77 | S |
| ATOM | 12897 | C | CYS | G | 62 | −8.174 | 10.801 | 86.686 | 1.00 | 62.95 | C |
| ATOM | 12898 | O | CYS | G | 62 | −9.048 | 10.565 | 87.488 | 1.00 | 64.78 | O |
| ATOM | 12899 | N | ASP | G | 63 | −7.131 | 10.003 | 86.543 | 1.00 | 64.90 | N |
| ATOM | 12901 | CA | ASP | G | 63 | −7.003 | 8.758 | 87.289 | 1.00 | 68.64 | C |
| ATOM | 12903 | CB | ASP | G | 63 | −5.755 | 7.993 | 86.843 | 1.00 | 72.80 | C |
| ATOM | 12906 | CG | ASP | G | 63 | −5.824 | 7.540 | 85.380 | 1.00 | 83.05 | C |
| ATOM | 12907 | OD1 | ASP | G | 63 | −6.831 | 7.773 | 84.664 | 1.00 | 83.68 | O |
| ATOM | 12908 | OD2 | ASP | G | 63 | −4.869 | 6.949 | 84.848 | 1.00 | 91.90 | O |
| ATOM | 12909 | C | ASP | G | 63 | −6.930 | 8.988 | 88.784 | 1.00 | 68.70 | C |
| ATOM | 12910 | O | ASP | G | 63 | −7.316 | 8.145 | 89.569 | 1.00 | 74.86 | O |
| ATOM | 12911 | N | ARG | G | 64 | −6.425 | 10.129 | 89.206 | 1.00 | 74.70 | N |
| ATOM | 12913 | CA | ARG | G | 64 | −6.124 | 10.304 | 90.614 | 1.00 | 74.37 | C |
| ATOM | 12915 | CB | ARG | G | 64 | −4.751 | 9.729 | 90.915 | 1.00 | 82.89 | C |
| ATOM | 12918 | CG | ARG | G | 64 | −4.247 | 9.822 | 92.352 | 1.00 | 101.98 | C |
| ATOM | 12921 | CD | ARG | G | 64 | −2.860 | 9.178 | 92.459 | 1.00 | 121.79 | C |
| ATOM | 12924 | NE | ARG | G | 64 | −2.429 | 8.734 | 91.127 | 1.00 | 134.11 | N |
| ATOM | 12926 | CZ | ARG | G | 64 | −1.965 | 7.528 | 90.815 | 1.00 | 138.38 | C |
| ATOM | 12927 | NH1 | ARG | G | 64 | −1.795 | 6.601 | 91.748 | 1.00 | 144.63 | N |
| ATOM | 12930 | NH2 | ARG | G | 64 | −1.640 | 7.252 | 89.557 | 1.00 | 137.56 | N |
| ATOM | 12933 | C | ARG | G | 64 | −6.131 | 11.792 | 90.829 | 1.00 | 70.07 | C |
| ATOM | 12934 | O | ARG | G | 64 | −5.561 | 12.550 | 90.047 | 1.00 | 69.25 | O |
| ATOM | 12935 | N | ASN | G | 65 | −6.899 | 12.189 | 91.829 | 1.00 | 61.92 | N |
| ATOM | 12937 | CA | ASN | G | 65 | −6.731 | 13.463 | 92.474 | 1.00 | 53.45 | C |
| ATOM | 12939 | CB | ASN | G | 65 | −7.357 | 13.294 | 93.841 | 1.00 | 47.72 | C |
| ATOM | 12942 | CG | ASN | G | 65 | −8.867 | 13.367 | 93.785 | 1.00 | 43.77 | C |
| ATOM | 12943 | OD1 | ASN | G | 65 | −9.541 | 13.528 | 94.805 | 1.00 | 58.79 | O |
| ATOM | 12944 | ND2 | ASN | G | 65 | −9.405 | 13.371 | 92.580 | 1.00 | 54.20 | N |
| ATOM | 12947 | C | ASN | G | 65 | −5.265 | 13.937 | 92.526 | 1.00 | 57.79 | C |
| ATOM | 12948 | O | ASN | G | 65 | −4.403 | 13.275 | 93.126 | 1.00 | 62.63 | O |
| ATOM | 12949 | N | LEU | G | 66 | −4.970 | 15.035 | 91.825 | 1.00 | 53.94 | N |
| ATOM | 12951 | CA | LEU | G | 66 | −3.739 | 15.796 | 92.037 | 1.00 | 49.89 | C |
| ATOM | 12953 | CB | LEU | G | 66 | −3.126 | 16.221 | 90.719 | 1.00 | 51.74 | C |
| ATOM | 12956 | CG | LEU | G | 66 | −2.792 | 15.242 | 89.611 | 1.00 | 60.32 | C |
| ATOM | 12958 | CD1 | LEU | G | 66 | −3.165 | 15.940 | 88.318 | 1.00 | 81.92 | C |
| ATOM | 12962 | CD2 | LEU | G | 66 | −1.306 | 14.907 | 89.588 | 1.00 | 74.82 | C |
| ATOM | 12966 | C | LEU | G | 66 | −4.005 | 17.101 | 92.771 | 1.00 | 49.02 | C |
| ATOM | 12967 | O | LEU | G | 66 | −5.054 | 17.738 | 92.591 | 1.00 | 51.20 | O |
| ATOM | 12968 | N | ALA | G | 67 | −3.010 | 17.532 | 93.535 | 1.00 | 46.28 | N |
| ATOM | 12970 | CA | ALA | G | 67 | −3.009 | 18.888 | 94.066 | 1.00 | 54.02 | C |
| ATOM | 12972 | CB | ALA | G | 67 | −3.077 | 18.844 | 95.572 | 1.00 | 52.72 | C |
| ATOM | 12976 | C | ALA | G | 67 | −1.777 | 19.662 | 93.598 | 1.00 | 51.71 | C |
| ATOM | 12977 | O | ALA | G | 67 | −0.743 | 19.653 | 94.273 | 1.00 | 51.50 | O |
| ATOM | 12978 | N | MET | G | 68 | −1.889 | 20.314 | 92.442 | 1.00 | 46.28 | N |
| ATOM | 12980 | CA | MET | G | 68 | −0.736 | 20.949 | 91.811 | 1.00 | 45.97 | C |
| ATOM | 12982 | CB | MET | G | 68 | −0.888 | 20.999 | 90.290 | 1.00 | 49.72 | C |
| ATOM | 12985 | CG | MET | G | 68 | −0.291 | 19.802 | 89.552 | 1.00 | 49.75 | C |
| ATOM | 12988 | SD | MET | G | 68 | −0.976 | 19.519 | 87.928 | 1.00 | 55.48 | S |
| ATOM | 12989 | CE | MET | G | 68 | −1.718 | 21.249 | 87.582 | 1.00 | 43.55 | C |
| ATOM | 12993 | C | MET | G | 68 | −0.416 | 22.352 | 92.328 | 1.00 | 49.00 | C |
| ATOM | 12994 | O | MET | G | 68 | −1.254 | 23.279 | 92.372 | 1.00 | 48.31 | O |
| ATOM | 12995 | N | GLY | G | 69 | 0.845 | 22.482 | 92.717 | 1.00 | 48.34 | N |
| ATOM | 12997 | CA | GLY | G | 69 | 1.366 | 23.741 | 93.192 | 1.00 | 47.90 | C |
| ATOM | 13000 | C | GLY | G | 69 | 1.990 | 24.462 | 92.021 | 1.00 | 51.01 | C |
| ATOM | 13001 | O | GLY | G | 69 | 3.034 | 24.031 | 91.475 | 1.00 | 41.59 | O |
| ATOM | 13002 | N | VAL | G | 70 | 1.352 | 25.584 | 91.692 | 1.00 | 45.06 | N |
| ATOM | 13004 | CA | VAL | G | 70 | 1.647 | 26.300 | 90.477 | 1.00 | 44.98 | C |
| ATOM | 13006 | CB | VAL | G | 70 | 0.411 | 26.436 | 89.568 | 1.00 | 46.39 | C |
| ATOM | 13008 | CG1 | VAL | G | 70 | 0.700 | 27.405 | 88.484 | 1.00 | 50.66 | C |
| ATOM | 13012 | CG2 | VAL | G | 70 | 0.022 | 25.118 | 88.935 | 1.00 | 51.15 | C |
| ATOM | 13016 | C | VAL | G | 70 | 2.074 | 27.688 | 90.863 | 1.00 | 45.27 | C |
| ATOM | 13017 | O | VAL | G | 70 | 1.396 | 28.345 | 91.659 | 1.00 | 42.89 | O |
| ATOM | 13018 | N | ASN | G | 71 | 3.165 | 28.126 | 90.233 | 1.00 | 46.78 | N |
| ATOM | 13020 | CA | ASN | G | 71 | 3.508 | 29.536 | 90.116 | 1.00 | 39.63 | C |
| ATOM | 13022 | CB | ASN | G | 71 | 5.024 | 29.729 | 89.961 | 1.00 | 43.27 | C |
| ATOM | 13025 | CG | ASN | G | 71 | 5.419 | 31.188 | 89.709 | 1.00 | 47.42 | C |
| ATOM | 13026 | OD1 | ASN | G | 71 | 5.010 | 31.797 | 88.719 | 1.00 | 61.72 | O |
| ATOM | 13027 | ND2 | ASN | G | 71 | 6.221 | 31.746 | 90.593 | 1.00 | 49.91 | N |
| ATOM | 13030 | C | ASN | G | 71 | 2.784 | 30.192 | 88.955 | 1.00 | 44.74 | C |
| ATOM | 13031 | O | ASN | G | 71 | 3.030 | 29.921 | 87.759 | 1.00 | 49.98 | O |
| ATOM | 13032 | N | LEU | G | 72 | 1.940 | 31.146 | 89.310 | 1.00 | 37.58 | N |
| ATOM | 13034 | CA | LEU | G | 72 | 1.079 | 31.721 | 88.301 | 1.00 | 35.90 | C |
| ATOM | 13036 | CB | LEU | G | 72 | −0.163 | 32.334 | 88.923 | 1.00 | 31.75 | C |
| ATOM | 13039 | CG | LEU | G | 72 | −1.024 | 31.209 | 89.506 | 1.00 | 36.57 | C |
| ATOM | 13041 | CD1 | LEU | G | 72 | −1.977 | 31.797 | 90.510 | 1.00 | 61.49 | C |
| ATOM | 13045 | CD2 | LEU | G | 72 | −1.810 | 30.570 | 88.414 | 1.00 | 59.43 | C |
| ATOM | 13049 | C | LEU | G | 72 | 1.796 | 32.651 | 87.348 | 1.00 | 35.89 | C |
| ATOM | 13050 | O | LEU | G | 72 | 1.469 | 32.737 | 86.174 | 1.00 | 45.71 | O |
| ATOM | 13051 | N | THR | G | 73 | 2.821 | 33.340 | 87.807 | 1.00 | 47.96 | N |
| ATOM | 13053 | CA | THR | G | 73 | 3.593 | 34.102 | 86.837 | 1.00 | 50.23 | C |
| ATOM | 13055 | CB | THR | G | 73 | 4.639 | 35.001 | 87.521 | 1.00 | 53.13 | C |
| ATOM | 13057 | OG1 | THR | G | 73 | 4.185 | 35.343 | 88.838 | 1.00 | 70.34 | O |
| ATOM | 13059 | CG2 | THR | G | 73 | 4.725 | 36.345 | 86.838 | 1.00 | 56.26 | C |
| ATOM | 13063 | C | THR | G | 73 | 4.208 | 33.096 | 85.867 | 1.00 | 46.02 | C |
| ATOM | 13064 | O | THR | G | 73 | 4.089 | 33.264 | 84.678 | 1.00 | 39.16 | O |
| ATOM | 13065 | N | SER | G | 74 | 4.789 | 31.987 | 86.305 | 1.00 | 42.72 | N |
| ATOM | 13067 | CA | SER | G | 74 | 5.444 | 31.184 | 85.283 | 1.00 | 45.45 | C |
| ATOM | 13069 | CB | SER | G | 74 | 6.275 | 30.062 | 85.893 | 1.00 | 46.41 | C |
| ATOM | 13072 | OG | SER | G | 74 | 7.141 | 30.498 | 86.919 | 1.00 | 55.46 | O |
| ATOM | 13074 | C | SER | G | 74 | 4.420 | 30.606 | 84.292 | 1.00 | 46.41 | C |
| ATOM | 13075 | O | SER | G | 74 | 4.674 | 30.497 | 83.120 | 1.00 | 44.10 | O |
| ATOM | 13076 | N | MET | G | 75 | 3.270 | 30.154 | 84.759 | 1.00 | 50.55 | N |
| ATOM | 13078 | CA | MET | G | 75 | 2.308 | 29.545 | 83.855 | 1.00 | 48.95 | C |
| ATOM | 13080 | CB | MET | G | 75 | 1.225 | 28.915 | 84.733 | 1.00 | 47.65 | C |
| ATOM | 13083 | CG | MET | G | 75 | 0.148 | 28.154 | 83.998 | 1.00 | 47.29 | C |
| ATOM | 13086 | SD | MET | G | 75 | −1.173 | 27.479 | 85.082 | 1.00 | 46.29 | S |
| ATOM | 13087 | CE | MET | G | 75 | −1.589 | 25.961 | 84.110 | 1.00 | 37.69 | C |
| ATOM | 13091 | C | MET | G | 75 | 1.714 | 30.581 | 82.862 | 1.00 | 45.86 | C |
| ATOM | 13092 | O | MET | G | 75 | 1.456 | 30.310 | 81.688 | 1.00 | 43.19 | O |
| ATOM | 13093 | N | SER | G | 76 | 1.501 | 31.793 | 83.340 | 1.00 | 33.72 | N |
| ATOM | 13095 | CA | SER | G | 76 | 1.206 | 32.909 | 82.477 | 1.00 | 34.81 | C |
| ATOM | 13097 | CB | SER | G | 76 | 1.074 | 34.181 | 83.345 | 1.00 | 39.63 | C |
| ATOM | 13100 | OG | SER | G | 76 | 1.060 | 35.353 | 82.528 | 1.00 | 61.17 | O |
| ATOM | 13102 | C | SER | G | 76 | 2.279 | 33.092 | 81.384 | 1.00 | 34.05 | C |
| ATOM | 13103 | O | SER | G | 76 | 2.002 | 33.231 | 80.203 | 1.00 | 37.60 | O |
| ATOM | 13104 | N | LYS | G | 77 | 3.545 | 33.129 | 81.758 | 1.00 | 42.20 | N |
| ATOM | 13106 | CA | LYS | G | 77 | 4.602 | 33.262 | 80.759 | 1.00 | 40.85 | C |
| ATOM | 13108 | CB | LYS | G | 77 | 6.002 | 33.158 | 81.388 | 1.00 | 43.48 | C |
| ATOM | 13111 | CG | LYS | G | 77 | 6.427 | 34.360 | 82.273 | 1.00 | 51.00 | C |
| ATOM | 13114 | CD | LYS | G | 77 | 7.905 | 34.254 | 82.668 | 1.00 | 54.33 | C |
| ATOM | 13117 | CE | LYS | G | 77 | 8.365 | 35.399 | 83.533 | 1.00 | 55.56 | C |
| ATOM | 13120 | NZ | LYS | G | 77 | 8.639 | 34.923 | 84.915 | 1.00 | 72.12 | N |
| ATOM | 13124 | C | LYS | G | 77 | 4.433 | 32.159 | 79.728 | 1.00 | 35.27 | C |
| ATOM | 13125 | O | LYS | G | 77 | 4.536 | 32.414 | 78.543 | 1.00 | 42.95 | O |
| ATOM | 13126 | N | ILE | G | 78 | 4.200 | 30.934 | 80.165 | 1.00 | 31.04 | N |
| ATOM | 13128 | CA | ILE | G | 78 | 4.012 | 29.858 | 79.218 | 1.00 | 37.77 | C |
| ATOM | 13130 | CB | ILE | G | 78 | 3.995 | 28.496 | 79.891 | 1.00 | 44.36 | C |
| ATOM | 13132 | CG1 | ILE | G | 78 | 5.376 | 28.161 | 80.459 | 1.00 | 33.05 | C |
| ATOM | 13135 | CD1 | ILE | G | 78 | 5.273 | 27.048 | 81.415 | 1.00 | 42.93 | C |
| ATOM | 13139 | CG2 | ILE | G | 78 | 3.669 | 27.438 | 78.877 | 1.00 | 51.07 | C |
| ATOM | 13143 | C | ILE | G | 78 | 2.772 | 30.045 | 78.389 | 1.00 | 35.52 | C |
| ATOM | 13144 | O | ILE | G | 78 | 2.852 | 29.972 | 77.170 | 1.00 | 37.88 | O |
| ATOM | 13145 | N | LEU | G | 79 | 1.648 | 30.388 | 79.006 | 1.00 | 32.30 | N |
| ATOM | 13147 | CA | LEU | G | 79 | 0.427 | 30.557 | 78.202 | 1.00 | 36.41 | C |
| ATOM | 13149 | CB | LEU | G | 79 | −0.838 | 30.610 | 79.071 | 1.00 | 26.56 | C |
| ATOM | 13152 | CG | LEU | G | 79 | −1.056 | 29.268 | 79.786 | 1.00 | 35.81 | C |
| ATOM | 13154 | CD1 | LEU | G | 79 | −1.966 | 29.399 | 80.979 | 1.00 | 60.89 | C |
| ATOM | 13158 | CD2 | LEU | G | 79 | −1.582 | 28.216 | 78.879 | 1.00 | 37.03 | C |
| ATOM | 13162 | C | LEU | G | 79 | 0.454 | 31.744 | 77.245 | 1.00 | 37.40 | C |
| ATOM | 13163 | O | LEU | G | 79 | −0.349 | 31.849 | 76.328 | 1.00 | 43.42 | O |
| ATOM | 13164 | N | LYS | G | 80 | 1.329 | 32.699 | 77.514 | 1.00 | 42.59 | N |
| ATOM | 13166 | CA | LYS | G | 80 | 1.481 | 33.820 | 76.617 | 1.00 | 40.17 | C |
| ATOM | 13168 | CB | LYS | G | 80 | 2.255 | 34.956 | 77.294 | 1.00 | 39.84 | C |
| ATOM | 13171 | CG | LYS | G | 80 | 1.253 | 35.878 | 78.023 | 1.00 | 42.88 | C |
| ATOM | 13174 | CD | LYS | G | 80 | 1.765 | 37.148 | 78.661 | 1.00 | 53.17 | C |
| ATOM | 13177 | CE | LYS | G | 80 | 0.887 | 38.349 | 78.289 | 1.00 | 65.36 | C |
| ATOM | 13180 | NZ | LYS | G | 80 | 1.165 | 39.497 | 79.221 | 1.00 | 74.40 | N |
| ATOM | 13184 | C | LYS | G | 80 | 2.096 | 33.337 | 75.326 | 1.00 | 43.07 | C |
| ATOM | 13185 | O | LYS | G | 80 | 2.001 | 34.025 | 74.342 | 1.00 | 45.70 | O |
| ATOM | 13186 | N | CYS | G | 81 | 2.604 | 32.103 | 75.307 | 1.00 | 53.10 | N |
| ATOM | 13188 | CA | CYS | G | 81 | 3.190 | 31.495 | 74.099 | 1.00 | 54.03 | C |
| ATOM | 13190 | CB | CYS | G | 81 | 4.255 | 30.427 | 74.419 | 1.00 | 48.26 | C |
| ATOM | 13193 | SG | CYS | G | 81 | 5.695 | 31.081 | 75.245 | 1.00 | 45.12 | S |
| ATOM | 13194 | C | CYS | G | 81 | 2.174 | 30.762 | 73.264 | 1.00 | 50.65 | C |
| ATOM | 13195 | O | CYS | G | 81 | 2.569 | 30.136 | 72.292 | 1.00 | 56.98 | O |
| ATOM | 13196 | N | ALA | G | 82 | 0.942 | 30.661 | 73.743 | 1.00 | 51.66 | N |
| ATOM | 13198 | CA | ALA | G | 82 | −0.118 | 30.021 | 72.959 | 1.00 | 53.78 | C |
| ATOM | 13200 | CB | ALA | G | 82 | −1.043 | 29.206 | 73.819 | 1.00 | 52.80 | C |
| ATOM | 13204 | C | ALA | G | 82 | −0.914 | 31.117 | 72.321 | 1.00 | 49.05 | C |
| ATOM | 13205 | O | ALA | G | 82 | −1.205 | 32.122 | 72.972 | 1.00 | 51.03 | O |
| ATOM | 13206 | N | GLY | G | 83 | −1.275 | 30.922 | 71.060 | 1.00 | 49.15 | N |
| ATOM | 13208 | CA | GLY | G | 83 | −2.139 | 31.891 | 70.406 | 1.00 | 47.99 | C |
| ATOM | 13211 | C | GLY | G | 83 | −3.568 | 31.653 | 70.843 | 1.00 | 50.67 | C |
| ATOM | 13212 | O | GLY | G | 83 | −3.895 | 30.606 | 71.417 | 1.00 | 44.78 | O |
| ATOM | 13213 | N | ASN | G | 84 | −4.436 | 32.597 | 70.506 | 1.00 | 52.66 | N |
| ATOM | 13215 | CA | ASN | G | 84 | −5.846 | 32.532 | 70.885 | 1.00 | 55.68 | C |
| ATOM | 13217 | CB | ASN | G | 84 | −6.473 | 33.863 | 70.523 | 1.00 | 61.31 | C |
| ATOM | 13220 | CG | ASN | G | 84 | −5.951 | 34.953 | 71.405 | 1.00 | 60.01 | C |
| ATOM | 13221 | OD1 | ASN | G | 84 | −6.149 | 34.899 | 72.620 | 1.00 | 53.17 | O |
| ATOM | 13222 | ND2 | ASN | G | 84 | −5.109 | 35.806 | 70.847 | 1.00 | 74.24 | N |
| ATOM | 13225 | C | ASN | G | 84 | −6.703 | 31.399 | 70.348 | 1.00 | 55.50 | C |
| ATOM | 13226 | O | ASN | G | 84 | −7.622 | 30.941 | 71.036 | 1.00 | 60.30 | O |
| ATOM | 13227 | N | GLU | G | 85 | −6.348 | 30.906 | 69.162 | 1.00 | 56.60 | N |
| ATOM | 13229 | CA | GLU | G | 85 | −6.981 | 29.732 | 68.574 | 1.00 | 53.85 | C |
| ATOM | 13231 | CB | GLU | G | 85 | −7.122 | 30.000 | 67.083 | 1.00 | 59.67 | C |
| ATOM | 13234 | CG | GLU | G | 85 | −7.747 | 31.360 | 66.769 | 1.00 | 78.12 | C |
| ATOM | 13237 | CD | GLU | G | 85 | −9.145 | 31.516 | 67.353 | 1.00 | 90.27 | C |
| ATOM | 13238 | OE1 | GLU | G | 85 | −9.417 | 32.574 | 67.953 | 1.00 | 105.63 | O |
| ATOM | 13239 | OE2 | GLU | G | 85 | −9.982 | 30.591 | 67.224 | 1.00 | 101.32 | O |
| ATOM | 13240 | C | GLU | G | 85 | −6.296 | 28.372 | 68.829 | 1.00 | 54.01 | C |
| ATOM | 13241 | O | GLU | G | 85 | −6.781 | 27.331 | 68.388 | 1.00 | 57.64 | O |
| ATOM | 13242 | N | ASP | G | 86 | −5.170 | 28.348 | 69.531 | 1.00 | 49.62 | N |
| ATOM | 13244 | CA | ASP | G | 86 | −4.526 | 27.087 | 69.845 | 1.00 | 44.35 | C |
| ATOM | 13246 | CB | ASP | G | 86 | −3.187 | 27.331 | 70.514 | 1.00 | 48.69 | C |
| ATOM | 13249 | CG | ASP | G | 86 | −2.141 | 27.869 | 69.562 | 1.00 | 55.97 | C |
| ATOM | 13250 | OD1 | ASP | G | 86 | −1.049 | 28.233 | 70.047 | 1.00 | 51.08 | O |
| ATOM | 13251 | OD2 | ASP | G | 86 | −2.298 | 27.939 | 68.327 | 1.00 | 71.92 | O |
| ATOM | 13252 | C | ASP | G | 86 | −5.377 | 26.164 | 70.719 | 1.00 | 44.20 | C |
| ATOM | 13253 | O | ASP | G | 86 | −5.941 | 26.543 | 71.711 | 1.00 | 47.71 | O |
| ATOM | 13254 | N | ILE | G | 87 | −5.539 | 24.924 | 70.302 | 1.00 | 50.59 | N |
| ATOM | 13256 | CA | ILE | G | 87 | −6.045 | 23.891 | 71.173 | 1.00 | 46.85 | C |
| ATOM | 13258 | CB | ILE | G | 87 | −6.212 | 22.656 | 70.278 | 1.00 | 54.80 | C |
| ATOM | 13260 | CG1 | ILE | G | 87 | −7.170 | 22.969 | 69.143 | 1.00 | 64.67 | C |
| ATOM | 13263 | CD1 | ILE | G | 87 | −6.665 | 22.368 | 67.860 | 1.00 | 88.91 | C |
| ATOM | 13267 | CG2 | ILE | G | 87 | −6.691 | 21.403 | 71.018 | 1.00 | 49.92 | C |
| ATOM | 13271 | C | ILE | G | 87 | −4.957 | 23.648 | 72.208 | 1.00 | 49.00 | C |
| ATOM | 13272 | O | ILE | G | 87 | −3.810 | 23.393 | 71.835 | 1.00 | 52.60 | O |
| ATOM | 13273 | N | ILE | G | 88 | −5.290 | 23.709 | 73.494 | 1.00 | 42.83 | N |
| ATOM | 13275 | CA | ILE | G | 88 | −4.276 | 23.577 | 74.535 | 1.00 | 39.24 | C |
| ATOM | 13277 | CB | ILE | G | 88 | −4.391 | 24.735 | 75.527 | 1.00 | 38.18 | C |
| ATOM | 13279 | CG1 | ILE | G | 88 | −4.041 | 26.056 | 74.848 | 1.00 | 36.21 | C |
| ATOM | 13282 | CD1 | ILE | G | 88 | −4.053 | 27.259 | 75.803 | 1.00 | 40.91 | C |
| ATOM | 13286 | CG2 | ILE | G | 88 | −3.448 | 24.516 | 76.701 | 1.00 | 46.27 | C |
| ATOM | 13290 | C | ILE | G | 88 | −4.487 | 22.273 | 75.274 | 1.00 | 41.66 | C |
| ATOM | 13291 | O | ILE | G | 88 | −5.614 | 21.903 | 75.558 | 1.00 | 44.54 | O |
| ATOM | 13292 | N | THR | G | 89 | −3.431 | 21.560 | 75.629 | 1.00 | 45.11 | N |
| ATOM | 13294 | CA | THR | G | 89 | −3.662 | 20.285 | 76.291 | 1.00 | 43.34 | C |
| ATOM | 13296 | CB | THR | G | 89 | −3.352 | 19.108 | 75.360 | 1.00 | 46.48 | C |
| ATOM | 13298 | OG1 | THR | G | 89 | −4.202 | 19.124 | 74.203 | 1.00 | 43.13 | O |
| ATOM | 13300 | CG2 | THR | G | 89 | −3.695 | 17.820 | 76.047 | 1.00 | 49.78 | C |
| ATOM | 13304 | C | THR | G | 89 | −2.820 | 20.164 | 77.539 | 1.00 | 45.69 | C |
| ATOM | 13305 | O | THR | G | 89 | −1.591 | 20.159 | 77.485 | 1.00 | 50.47 | O |
| ATOM | 13306 | N | LEU | G | 90 | −3.478 | 20.000 | 78.673 | 1.00 | 43.24 | N |
| ATOM | 13308 | CA | LEU | G | 90 | −2.748 | 19.813 | 79.913 | 1.00 | 44.75 | C |
| ATOM | 13310 | CB | LEU | G | 90 | −3.552 | 20.419 | 81.057 | 1.00 | 35.43 | C |
| ATOM | 13313 | CG | LEU | G | 90 | −3.866 | 21.876 | 80.751 | 1.00 | 39.44 | C |
| ATOM | 13315 | CD1 | LEU | G | 90 | −4.647 | 22.527 | 81.904 | 1.00 | 35.65 | C |
| ATOM | 13319 | CD2 | LEU | G | 90 | −2.573 | 22.610 | 80.512 | 1.00 | 32.17 | C |
| ATOM | 13323 | C | LEU | G | 90 | −2.447 | 18.345 | 80.188 | 1.00 | 45.36 | C |
| ATOM | 13324 | O | LEU | G | 90 | −3.312 | 17.496 | 80.007 | 1.00 | 41.87 | O |
| ATOM | 13325 | N | ARG | G | 91 | −1.253 | 18.044 | 80.696 | 1.00 | 48.57 | N |
| ATOM | 13327 | CA | ARG | G | 91 | −0.911 | 16.643 | 80.979 | 1.00 | 51.24 | C |
| ATOM | 13329 | CB | ARG | G | 91 | −0.236 | 15.986 | 79.763 | 1.00 | 52.72 | C |
| ATOM | 13332 | CG | ARG | G | 91 | −0.187 | 14.453 | 79.793 | 1.00 | 63.85 | C |
| ATOM | 13335 | CD | ARG | G | 91 | −0.526 | 13.785 | 78.452 | 1.00 | 80.54 | C |
| ATOM | 13338 | NE | ARG | G | 91 | 0.339 | 14.220 | 77.353 | 1.00 | 100.42 | N |
| ATOM | 13340 | CZ | ARG | G | 91 | −0.056 | 14.903 | 76.276 | 1.00 | 115.81 | C |
| ATOM | 13341 | NH1 | ARG | G | 91 | −1.316 | 15.290 | 76.122 | 1.00 | 124.13 | N |
| ATOM | 13344 | NH2 | ARG | G | 91 | 0.824 | 15.211 | 75.333 | 1.00 | 124.48 | N |
| ATOM | 13347 | C | ARG | G | 91 | 0.029 | 16.493 | 82.157 | 1.00 | 43.41 | C |
| ATOM | 13348 | O | ARG | G | 91 | 1.116 | 17.028 | 82.077 | 1.00 | 45.31 | O |
| ATOM | 13349 | N | ALA | G | 92 | −0.344 | 15.675 | 83.151 | 1.00 | 45.40 | N |
| ATOM | 13351 | CA | ALA | G | 92 | 0.408 | 15.454 | 84.404 | 1.00 | 47.10 | C |
| ATOM | 13353 | CB | ALA | G | 92 | −0.196 | 16.273 | 85.521 | 1.00 | 45.12 | C |
| ATOM | 13357 | C | ALA | G | 92 | 0.403 | 13.996 | 84.854 | 1.00 | 55.98 | C |
| ATOM | 13358 | O | ALA | G | 92 | −0.677 | 13.460 | 85.057 | 1.00 | 70.09 | O |
| ATOM | 13359 | N | GLU | G | 93 | 1.566 | 13.372 | 85.066 | 1.00 | 65.85 | N |
| ATOM | 13361 | CA | GLU | G | 93 | 1.649 | 12.076 | 85.756 | 1.00 | 69.59 | C |
| ATOM | 13363 | CB | GLU | G | 93 | 3.027 | 11.408 | 85.631 | 1.00 | 69.24 | C |
| ATOM | 13366 | CG | GLU | G | 93 | 3.613 | 11.332 | 84.229 | 1.00 | 76.17 | C |
| ATOM | 13369 | CD | GLU | G | 93 | 3.054 | 10.187 | 83.392 | 1.00 | 98.68 | C |
| ATOM | 13370 | OE1 | GLU | G | 93 | 3.494 | 9.027 | 83.579 | 1.00 | 108.43 | O |
| ATOM | 13371 | OE2 | GLU | G | 93 | 2.199 | 10.444 | 82.514 | 1.00 | 91.56 | O |
| ATOM | 13372 | C | GLU | G | 93 | 1.392 | 12.414 | 87.210 | 1.00 | 76.08 | C |
| ATOM | 13373 | O | GLU | G | 93 | 1.271 | 13.595 | 87.542 | 1.00 | 70.65 | O |
| ATOM | 13374 | N | ASP | G | 94 | 1.294 | 11.400 | 88.066 | 1.00 | 87.86 | N |
| ATOM | 13376 | CA | ASP | G | 94 | 0.483 | 11.528 | 89.281 | 1.00 | 97.13 | C |
| ATOM | 13378 | CB | ASP | G | 94 | −0.477 | 10.341 | 89.430 | 1.00 | 94.62 | C |
| ATOM | 13381 | CG | ASP | G | 94 | −0.955 | 9.794 | 88.079 | 1.00 | 112.68 | C |
| ATOM | 13382 | OD1 | ASP | G | 94 | −2.085 | 10.151 | 87.650 | 1.00 | 113.50 | O |
| ATOM | 13383 | OD2 | ASP | G | 94 | −0.266 | 9.001 | 87.382 | 1.00 | 117.83 | O |
| ATOM | 13384 | C | ASP | G | 94 | 1.347 | 11.746 | 90.537 | 1.00 | 102.84 | C |
| ATOM | 13385 | O | ASP | G | 94 | 1.035 | 12.596 | 91.387 | 1.00 | 103.66 | O |
| ATOM | 13386 | N | ASN | G | 95 | 2.456 | 11.010 | 90.615 | 1.00 | 105.07 | N |
| ATOM | 13388 | CA | ASN | G | 95 | 3.618 | 11.384 | 91.424 | 1.00 | 105.25 | C |
| ATOM | 13390 | CB | ASN | G | 95 | 4.646 | 10.229 | 91.429 | 1.00 | 107.15 | C |
| ATOM | 13393 | CG | ASN | G | 95 | 4.716 | 9.480 | 92.764 | 1.00 | 107.96 | C |
| ATOM | 13394 | OD1 | ASN | G | 95 | 5.767 | 8.948 | 93.125 | 1.00 | 110.77 | O |
| ATOM | 13395 | ND2 | ASN | G | 95 | 3.604 | 9.435 | 93.495 | 1.00 | 96.85 | N |
| ATOM | 13398 | C | ASN | G | 95 | 4.278 | 12.628 | 90.824 | 1.00 | 102.55 | C |
| ATOM | 13399 | O | ASN | G | 95 | 5.346 | 12.507 | 90.221 | 1.00 | 106.27 | O |
| ATOM | 13400 | N | ALA | G | 96 | 3.652 | 13.799 | 90.971 | 1.00 | 96.59 | N |
| ATOM | 13402 | CA | ALA | G | 96 | 3.583 | 14.785 | 89.880 | 1.00 | 91.29 | C |
| ATOM | 13404 | CB | ALA | G | 96 | 2.166 | 15.322 | 89.736 | 1.00 | 90.95 | C |
| ATOM | 13408 | C | ALA | G | 96 | 4.519 | 15.957 | 90.112 | 1.00 | 81.93 | C |
| ATOM | 13409 | O | ALA | G | 96 | 4.211 | 16.789 | 90.964 | 1.00 | 79.71 | O |
| ATOM | 13410 | N | ASP | G | 97 | 5.622 | 16.054 | 89.365 | 1.00 | 72.01 | N |
| ATOM | 13412 | CA | ASP | G | 97 | 6.529 | 17.184 | 89.578 | 1.00 | 67.13 | C |
| ATOM | 13414 | CB | ASP | G | 97 | 7.792 | 16.781 | 90.343 | 1.00 | 64.05 | C |
| ATOM | 13417 | CG | ASP | G | 97 | 7.571 | 16.895 | 91.850 | 1.00 | 81.67 | C |
| ATOM | 13418 | OD1 | ASP | G | 97 | 7.509 | 15.842 | 92.520 | 1.00 | 103.46 | O |
| ATOM | 13419 | OD2 | ASP | G | 97 | 7.299 | 17.972 | 92.443 | 1.00 | 89.15 | O |
| ATOM | 13420 | C | ASP | G | 97 | 6.752 | 18.181 | 88.451 | 1.00 | 54.65 | C |
| ATOM | 13421 | O | ASP | G | 97 | 7.405 | 19.196 | 88.614 | 1.00 | 51.47 | O |
| ATOM | 13422 | N | THR | G | 98 | 6.060 | 17.954 | 87.352 | 1.00 | 59.25 | N |
| ATOM | 13424 | CA | THR | G | 98 | 6.002 | 18.883 | 86.229 | 1.00 | 60.10 | C |
| ATOM | 13426 | CB | THR | G | 98 | 6.938 | 18.420 | 85.120 | 1.00 | 55.13 | C |
| ATOM | 13428 | OG1 | THR | G | 98 | 7.399 | 17.096 | 85.406 | 1.00 | 73.85 | O |
| ATOM | 13430 | CG2 | THR | G | 98 | 8.185 | 19.240 | 85.154 | 1.00 | 62.92 | C |
| ATOM | 13434 | C | THR | G | 98 | 4.631 | 18.798 | 85.636 | 1.00 | 56.24 | C |
| ATOM | 13435 | O | THR | G | 98 | 4.143 | 17.685 | 85.417 | 1.00 | 63.11 | O |
| ATOM | 13436 | N | LEU | G | 99 | 4.068 | 19.942 | 85.271 | 1.00 | 51.86 | N |
| ATOM | 13438 | CA | LEU | G | 99 | 2.928 | 19.950 | 84.349 | 1.00 | 46.26 | C |
| ATOM | 13440 | CB | LEU | G | 99 | 1.918 | 21.005 | 84.761 | 1.00 | 45.10 | C |
| ATOM | 13443 | CG | LEU | G | 99 | 0.653 | 21.073 | 83.919 | 1.00 | 49.07 | C |
| ATOM | 13445 | CD1 | LEU | G | 99 | −0.152 | 19.770 | 83.987 | 1.00 | 57.40 | C |
| ATOM | 13449 | CD2 | LEU | G | 99 | −0.191 | 22.230 | 84.419 | 1.00 | 35.41 | C |
| ATOM | 13453 | C | LEU | G | 99 | 3.342 | 20.240 | 82.924 | 1.00 | 46.53 | C |
| ATOM | 13454 | O | LEU | G | 99 | 4.121 | 21.165 | 82.650 | 1.00 | 47.47 | O |
| ATOM | 13455 | N | ALA | G | 100 | 2.767 | 19.459 | 82.015 | 1.00 | 48.92 | N |
| ATOM | 13457 | CA | ALA | G | 100 | 3.016 | 19.581 | 80.575 | 1.00 | 43.62 | C |
| ATOM | 13459 | CB | ALA | G | 100 | 3.158 | 18.227 | 79.951 | 1.00 | 38.24 | C |
| ATOM | 13463 | C | ALA | G | 100 | 1.879 | 20.329 | 79.911 | 1.00 | 43.41 | C |
| ATOM | 13464 | O | ALA | G | 100 | 0.729 | 20.220 | 80.335 | 1.00 | 42.91 | O |
| ATOM | 13465 | N | LEU | G | 101 | 2.215 | 21.116 | 78.896 | 1.00 | 45.53 | N |
| ATOM | 13467 | CA | LEU | G | 101 | 1.230 | 21.978 | 78.249 | 1.00 | 51.26 | C |
| ATOM | 13469 | CB | LEU | G | 101 | 1.283 | 23.435 | 78.732 | 1.00 | 51.69 | C |
| ATOM | 13472 | CG | LEU | G | 101 | 0.866 | 23.845 | 80.142 | 1.00 | 43.45 | C |
| ATOM | 13474 | CD1 | LEU | G | 101 | 2.125 | 24.090 | 80.881 | 1.00 | 51.34 | C |
| ATOM | 13478 | CD2 | LEU | G | 101 | 0.098 | 25.139 | 80.187 | 1.00 | 39.52 | C |
| ATOM | 13482 | C | LEU | G | 101 | 1.615 | 21.961 | 76.786 | 1.00 | 51.89 | C |
| ATOM | 13483 | O | LEU | G | 101 | 2.745 | 22.261 | 76.420 | 1.00 | 58.67 | O |
| ATOM | 13484 | N | VAL | G | 102 | 0.659 | 21.598 | 75.949 | 1.00 | 50.53 | N |
| ATOM | 13486 | CA | VAL | G | 102 | 0.910 | 21.370 | 74.537 | 1.00 | 43.29 | C |
| ATOM | 13488 | CB | VAL | G | 102 | 0.490 | 19.976 | 74.191 | 1.00 | 42.86 | C |
| ATOM | 13490 | CG1 | VAL | G | 102 | 0.813 | 19.672 | 72.751 | 1.00 | 50.76 | C |
| ATOM | 13494 | CG2 | VAL | G | 102 | 1.056 | 18.996 | 75.187 | 1.00 | 46.30 | C |
| ATOM | 13498 | C | VAL | G | 102 | −0.053 | 22.254 | 73.765 | 1.00 | 42.66 | C |
| ATOM | 13499 | O | VAL | G | 102 | −1.225 | 22.398 | 74.128 | 1.00 | 43.22 | O |
| ATOM | 13500 | N | PHE | G | 103 | 0.452 | 22.898 | 72.734 | 1.00 | 38.01 | N |
| ATOM | 13502 | CA | PHE | G | 103 | −0.302 | 23.954 | 72.105 | 1.00 | 43.04 | C |
| ATOM | 13504 | CB | PHE | G | 103 | 0.419 | 25.300 | 72.213 | 1.00 | 42.02 | C |
| ATOM | 13507 | CG | PHE | G | 103 | 0.525 | 25.837 | 73.589 | 1.00 | 39.34 | C |
| ATOM | 13508 | CD1 | PHE | G | 103 | −0.356 | 25.440 | 74.577 | 1.00 | 32.77 | C |
| ATOM | 13510 | CE1 | PHE | G | 103 | −0.229 | 25.953 | 75.862 | 1.00 | 48.87 | C |
| ATOM | 13512 | CZ | PHE | G | 103 | 0.779 | 26.873 | 76.146 | 1.00 | 52.37 | C |
| ATOM | 13514 | CE2 | PHE | G | 103 | 1.668 | 27.261 | 75.160 | 1.00 | 46.85 | C |
| ATOM | 13516 | CD2 | PHE | G | 103 | 1.500 | 26.788 | 73.882 | 1.00 | 51.33 | C |
| ATOM | 13518 | C | PHE | G | 103 | −0.320 | 23.569 | 70.644 | 1.00 | 44.87 | C |
| ATOM | 13519 | O | PHE | G | 103 | 0.703 | 23.690 | 69.963 | 1.00 | 53.09 | O |
| ATOM | 13520 | N | GLU | G | 104 | −1.481 | 23.189 | 70.136 | 1.00 | 48.89 | N |
| ATOM | 13522 | CA | GLU | G | 104 | −1.604 | 22.895 | 68.716 | 1.00 | 50.31 | C |
| ATOM | 13524 | CB | GLU | G | 104 | −2.458 | 21.662 | 68.513 | 1.00 | 50.28 | C |
| ATOM | 13527 | CG | GLU | G | 104 | −1.829 | 20.446 | 69.158 | 1.00 | 65.75 | C |
| ATOM | 13530 | CD | GLU | G | 104 | −2.438 | 19.177 | 68.620 | 1.00 | 81.96 | C |
| ATOM | 13531 | OE1 | GLU | G | 104 | −3.612 | 18.906 | 69.017 | 1.00 | 59.11 | O |
| ATOM | 13532 | OE2 | GLU | G | 104 | −1.747 | 18.543 | 67.766 | 1.00 | 62.21 | O |
| ATOM | 13533 | C | GLU | G | 104 | −2.231 | 24.074 | 68.008 | 1.00 | 51.96 | C |
| ATOM | 13534 | O | GLU | G | 104 | −3.387 | 24.404 | 68.247 | 1.00 | 57.10 | O |
| ATOM | 13535 | N | ALA | G | 105 | −1.445 | 24.695 | 67.139 | 1.00 | 53.36 | N |
| ATOM | 13537 | CA | ALA | G | 105 | −1.953 | 25.631 | 66.157 | 1.00 | 57.65 | C |
| ATOM | 13539 | CB | ALA | G | 105 | −0.863 | 25.964 | 65.153 | 1.00 | 58.43 | C |
| ATOM | 13543 | C | ALA | G | 105 | −3.141 | 25.031 | 65.435 | 1.00 | 62.75 | C |
| ATOM | 13544 | O | ALA | G | 105 | −3.092 | 23.903 | 64.964 | 1.00 | 64.39 | O |
| ATOM | 13545 | N | PRO | G | 106 | −4.181 | 25.820 | 65.231 | 1.00 | 77.01 | N |
| ATOM | 13546 | CA | PRO | G | 106 | −5.390 | 25.262 | 64.641 | 1.00 | 79.72 | C |
| ATOM | 13548 | CB | PRO | G | 106 | −6.466 | 26.211 | 65.151 | 1.00 | 77.58 | C |
| ATOM | 13551 | CG | PRO | G | 106 | −5.741 | 27.564 | 65.209 | 1.00 | 78.51 | C |
| ATOM | 13554 | CD | PRO | G | 106 | −4.255 | 27.291 | 65.331 | 1.00 | 76.52 | C |
| ATOM | 13557 | C | PRO | G | 106 | −5.155 | 25.318 | 63.130 | 1.00 | 81.94 | C |
| ATOM | 13558 | O | PRO | G | 106 | −5.276 | 24.263 | 62.531 | 1.00 | 83.54 | O |
| ATOM | 13559 | N | ASN | G | 107 | −4.673 | 26.431 | 62.571 | 1.00 | 89.50 | N |
| ATOM | 13561 | CA | ASN | G | 107 | −4.124 | 26.474 | 61.200 | 1.00 | 99.55 | C |
| ATOM | 13563 | CB | ASN | G | 107 | −3.303 | 27.761 | 60.966 | 1.00 | 102.78 | C |
| ATOM | 13566 | CG | ASN | G | 107 | −1.842 | 27.650 | 61.433 | 1.00 | 110.30 | C |
| ATOM | 13567 | OD1 | ASN | G | 107 | −1.058 | 26.817 | 60.938 | 1.00 | 87.61 | O |
| ATOM | 13568 | ND2 | ASN | G | 107 | −1.464 | 28.525 | 62.375 | 1.00 | 109.25 | N |
| ATOM | 13571 | C | ASN | G | 107 | −3.272 | 25.272 | 60.768 | 1.00 | 100.90 | C |
| ATOM | 13572 | O | ASN | G | 107 | −3.129 | 24.991 | 59.569 | 1.00 | 99.10 | O |
| ATOM | 13573 | N | GLN | G | 108 | −2.695 | 24.590 | 61.758 | 1.00 | 100.26 | N |
| ATOM | 13575 | CA | GLN | G | 108 | −1.760 | 23.491 | 61.542 | 1.00 | 93.80 | C |
| ATOM | 13577 | CB | GLN | G | 108 | −2.066 | 22.789 | 60.213 | 1.00 | 96.64 | C |
| ATOM | 13580 | CG | GLN | G | 108 | −2.103 | 21.275 | 60.307 | 1.00 | 100.20 | C |
| ATOM | 13583 | CD | GLN | G | 108 | −1.857 | 20.784 | 61.716 | 1.00 | 99.82 | C |
| ATOM | 13584 | OE1 | GLN | G | 108 | −2.820 | 20.563 | 62.453 | 1.00 | 106.60 | O |
| ATOM | 13585 | NE2 | GLN | G | 108 | −0.578 | 20.600 | 62.093 | 1.00 | 64.06 | N |
| ATOM | 13588 | C | GLN | G | 108 | −0.313 | 23.992 | 61.598 | 1.00 | 86.28 | C |
| ATOM | 13589 | O | GLN | G | 108 | −0.070 | 25.213 | 61.650 | 1.00 | 74.81 | O |
| ATOM | 13590 | N | GLU | G | 109 | 0.618 | 23.036 | 61.637 | 1.00 | 75.98 | N |
| ATOM | 13592 | CA | GLU | G | 109 | 1.944 | 23.164 | 61.035 | 1.00 | 76.16 | C |
| ATOM | 13594 | CB | GLU | G | 109 | 1.963 | 24.184 | 59.864 | 1.00 | 86.76 | C |
| ATOM | 13597 | CG | GLU | G | 109 | 1.595 | 25.657 | 60.166 | 1.00 | 104.93 | C |
| ATOM | 13600 | CD | GLU | G | 109 | 2.321 | 26.718 | 59.312 | 1.00 | 117.28 | C |
| ATOM | 13601 | OE1 | GLU | G | 109 | 2.205 | 26.724 | 58.061 | 1.00 | 108.14 | O |
| ATOM | 13602 | OE2 | GLU | G | 109 | 2.993 | 27.605 | 59.896 | 1.00 | 120.72 | O |
| ATOM | 13603 | C | GLU | G | 109 | 2.975 | 23.464 | 62.134 | 1.00 | 63.79 | C |
| ATOM | 13604 | O | GLU | G | 109 | 4.152 | 23.155 | 62.010 | 1.00 | 62.46 | O |
| ATOM | 13605 | N | LYS | G | 110 | 2.472 | 23.933 | 63.267 | 1.00 | 53.65 | N |
| ATOM | 13607 | CA | LYS | G | 110 | 3.253 | 24.230 | 64.470 | 1.00 | 50.22 | C |
| ATOM | 13609 | CB | LYS | G | 110 | 3.044 | 25.721 | 64.782 | 1.00 | 57.06 | C |
| ATOM | 13612 | CG | LYS | G | 110 | 4.155 | 26.488 | 65.465 | 1.00 | 46.78 | C |
| ATOM | 13615 | CD | LYS | G | 110 | 3.588 | 27.381 | 66.573 | 1.00 | 50.07 | C |
| ATOM | 13618 | CE | LYS | G | 110 | 3.810 | 28.876 | 66.258 | 1.00 | 69.40 | C |
| ATOM | 13621 | NZ | LYS | G | 110 | 4.412 | 29.732 | 67.332 | 1.00 | 35.33 | N |
| ATOM | 13625 | C | LYS | G | 110 | 2.698 | 23.455 | 65.672 | 1.00 | 51.79 | C |
| ATOM | 13626 | O | LYS | G | 110 | 1.493 | 23.438 | 65.941 | 1.00 | 43.91 | O |
| ATOM | 13627 | N | VAL | G | 111 | 3.585 | 22.853 | 66.452 | 1.00 | 60.07 | N |
| ATOM | 13629 | CA | VAL | G | 111 | 3.203 | 22.338 | 67.766 | 1.00 | 55.29 | C |
| ATOM | 13631 | CB | VAL | G | 111 | 3.179 | 20.814 | 67.837 | 1.00 | 47.96 | C |
| ATOM | 13633 | CG1 | VAL | G | 111 | 2.477 | 20.429 | 69.096 | 1.00 | 55.28 | C |
| ATOM | 13637 | CG2 | VAL | G | 111 | 2.476 | 20.230 | 66.653 | 1.00 | 55.28 | C |
| ATOM | 13641 | C | VAL | G | 111 | 4.234 | 22.719 | 68.785 | 1.00 | 52.39 | C |
| ATOM | 13642 | O | VAL | G | 111 | 5.436 | 22.567 | 68.546 | 1.00 | 55.27 | O |
| ATOM | 13643 | N | SER | G | 112 | 3.745 | 23.138 | 69.941 | 1.00 | 48.75 | N |
| ATOM | 13645 | CA | SER | G | 112 | 4.623 | 23.633 | 70.985 | 1.00 | 43.55 | C |
| ATOM | 13647 | CB | SER | G | 112 | 4.390 | 25.124 | 71.239 | 1.00 | 38.53 | C |
| ATOM | 13650 | OG | SER | G | 112 | 4.537 | 25.861 | 70.039 | 1.00 | 40.52 | O |
| ATOM | 13652 | C | SER | G | 112 | 4.382 | 22.817 | 72.241 | 1.00 | 45.21 | C |
| ATOM | 13653 | O | SER | G | 112 | 3.281 | 22.359 | 72.541 | 1.00 | 44.42 | O |
| ATOM | 13654 | N | ASP | G | 113 | 5.463 | 22.614 | 72.970 | 1.00 | 48.19 | N |
| ATOM | 13656 | CA | ASP | G | 113 | 5.489 | 21.625 | 74.026 | 1.00 | 53.42 | C |
| ATOM | 13658 | CB | ASP | G | 113 | 6.356 | 20.423 | 73.627 | 1.00 | 55.15 | C |
| ATOM | 13661 | CG | ASP | G | 113 | 5.537 | 19.292 | 73.067 | 1.00 | 64.69 | C |
| ATOM | 13662 | OD1 | ASP | G | 113 | 5.552 | 19.067 | 71.828 | 1.00 | 91.54 | O |
| ATOM | 13663 | OD2 | ASP | G | 113 | 4.819 | 18.596 | 73.813 | 1.00 | 76.23 | O |
| ATOM | 13664 | C | ASP | G | 113 | 6.167 | 22.394 | 75.127 | 1.00 | 49.36 | C |
| ATOM | 13665 | O | ASP | G | 113 | 7.275 | 22.882 | 74.955 | 1.00 | 54.19 | O |
| ATOM | 13666 | N | TYR | G | 114 | 5.498 | 22.526 | 76.250 | 1.00 | 42.68 | N |
| ATOM | 13668 | CA | TYR | G | 114 | 6.099 | 23.268 | 77.326 | 1.00 | 49.72 | C |
| ATOM | 13670 | CB | TYR | G | 114 | 5.422 | 24.620 | 77.575 | 1.00 | 45.42 | C |
| ATOM | 13673 | CG | TYR | G | 114 | 5.744 | 25.611 | 76.504 | 1.00 | 42.23 | C |
| ATOM | 13674 | CD1 | TYR | G | 114 | 6.944 | 26.300 | 76.501 | 1.00 | 59.79 | C |
| ATOM | 13676 | CE1 | TYR | G | 114 | 7.231 | 27.243 | 75.513 | 1.00 | 61.46 | C |
| ATOM | 13678 | CZ | TYR | G | 114 | 6.326 | 27.465 | 74.502 | 1.00 | 56.43 | C |
| ATOM | 13679 | OH | TYR | G | 114 | 6.576 | 28.367 | 73.508 | 1.00 | 44.18 | O |
| ATOM | 13681 | CE2 | TYR | G | 114 | 5.132 | 26.793 | 74.494 | 1.00 | 57.09 | C |
| ATOM | 13683 | CD2 | TYR | G | 114 | 4.859 | 25.848 | 75.472 | 1.00 | 38.06 | C |
| ATOM | 13685 | C | TYR | G | 114 | 5.947 | 22.393 | 78.530 | 1.00 | 51.14 | C |
| ATOM | 13686 | O | TYR | G | 114 | 4.927 | 21.734 | 78.693 | 1.00 | 50.48 | O |
| ATOM | 13687 | N | GLU | G | 115 | 6.944 | 22.486 | 79.394 | 1.00 | 49.02 | N |
| ATOM | 13689 | CA | GLU | G | 115 | 6.940 | 21.756 | 80.628 | 1.00 | 50.15 | C |
| ATOM | 13691 | CB | GLU | G | 115 | 7.935 | 20.621 | 80.479 | 1.00 | 45.87 | C |
| ATOM | 13694 | CG | GLU | G | 115 | 8.344 | 20.003 | 81.801 | 1.00 | 48.26 | C |
| ATOM | 13697 | CD | GLU | G | 115 | 9.427 | 18.962 | 81.618 | 1.00 | 65.88 | C |
| ATOM | 13698 | OE1 | GLU | G | 115 | 10.346 | 18.959 | 82.474 | 1.00 | 60.23 | O |
| ATOM | 13699 | OE2 | GLU | G | 115 | 9.379 | 18.195 | 80.616 | 1.00 | 68.84 | O |
| ATOM | 13700 | C | GLU | G | 115 | 7.308 | 22.688 | 81.782 | 1.00 | 46.41 | C |
| ATOM | 13701 | O | GLU | G | 115 | 8.373 | 23.283 | 81.793 | 1.00 | 48.23 | O |
| ATOM | 13702 | N | MET | G | 116 | 6.417 | 22.797 | 82.756 | 1.00 | 46.54 | N |
| ATOM | 13704 | CA | MET | G | 116 | 6.495 | 23.802 | 83.824 | 1.00 | 43.36 | C |
| ATOM | 13706 | CB | MET | G | 116 | 5.146 | 24.532 | 83.914 | 1.00 | 42.48 | C |
| ATOM | 13709 | CG | MET | G | 116 | 4.939 | 25.373 | 85.142 | 1.00 | 33.21 | C |
| ATOM | 13712 | SD | MET | G | 116 | 3.333 | 26.264 | 85.064 | 1.00 | 43.07 | S |
| ATOM | 13713 | CE | MET | G | 116 | 2.392 | 24.954 | 85.600 | 1.00 | 53.69 | C |
| ATOM | 13717 | C | MET | G | 116 | 6.761 | 23.157 | 85.182 | 1.00 | 38.99 | C |
| ATOM | 13718 | O | MET | G | 116 | 5.978 | 22.311 | 85.614 | 1.00 | 39.70 | O |
| ATOM | 13719 | N | LYS | G | 117 | 7.844 | 23.562 | 85.847 | 1.00 | 43.06 | N |
| ATOM | 13721 | CA | LYS | G | 117 | 8.213 | 23.006 | 87.161 | 1.00 | 42.52 | C |
| ATOM | 13723 | CB | LYS | G | 117 | 9.618 | 23.435 | 87.643 | 1.00 | 41.85 | C |
| ATOM | 13726 | CG | LYS | G | 117 | 10.790 | 22.786 | 86.856 | 1.00 | 50.59 | C |
| ATOM | 13729 | CD | LYS | G | 117 | 12.109 | 23.579 | 86.959 | 1.00 | 60.53 | C |
| ATOM | 13732 | CE | LYS | G | 117 | 13.246 | 23.073 | 86.041 | 1.00 | 82.93 | C |
| ATOM | 13735 | NZ | LYS | G | 117 | 14.269 | 22.152 | 86.681 | 1.00 | 96.88 | N |
| ATOM | 13739 | C | LYS | G | 117 | 7.144 | 23.455 | 88.126 | 1.00 | 37.27 | C |
| ATOM | 13740 | O | LYS | G | 117 | 6.683 | 24.580 | 88.032 | 1.00 | 44.66 | O |
| ATOM | 13741 | N | LEU | G | 118 | 6.623 | 22.509 | 88.896 | 1.00 | 40.33 | N |
| ATOM | 13743 | CA | LEU | G | 118 | 5.543 | 22.740 | 89.849 | 1.00 | 42.50 | C |
| ATOM | 13745 | CB | LEU | G | 118 | 4.689 | 21.482 | 90.076 | 1.00 | 41.49 | C |
| ATOM | 13748 | CG | LEU | G | 118 | 3.660 | 21.231 | 88.988 | 1.00 | 50.45 | C |
| ATOM | 13750 | CD1 | LEU | G | 118 | 3.262 | 19.778 | 88.969 | 1.00 | 44.46 | C |
| ATOM | 13754 | CD2 | LEU | G | 118 | 2.469 | 22.156 | 89.177 | 1.00 | 57.53 | C |
| ATOM | 13758 | C | LEU | G | 118 | 6.260 | 23.006 | 91.136 | 1.00 | 41.08 | C |
| ATOM | 13759 | O | LEU | G | 118 | 7.384 | 22.583 | 91.302 | 1.00 | 38.25 | O |
| ATOM | 13760 | N | MET | G | 119 | 5.578 | 23.602 | 92.094 | 1.00 | 45.12 | N |
| ATOM | 13762 | CA | MET | G | 119 | 6.287 | 23.990 | 93.279 | 1.00 | 47.45 | C |
| ATOM | 13764 | CB | MET | G | 119 | 6.421 | 25.514 | 93.288 | 1.00 | 52.91 | C |
| ATOM | 13767 | CG | MET | G | 119 | 5.094 | 26.217 | 93.271 | 1.00 | 59.54 | C |
| ATOM | 13770 | SD | MET | G | 119 | 5.269 | 27.982 | 93.385 | 1.00 | 73.29 | S |
| ATOM | 13771 | CE | MET | G | 119 | 5.380 | 28.295 | 95.169 | 1.00 | 71.07 | C |
| ATOM | 13775 | C | MET | G | 119 | 5.543 | 23.397 | 94.462 | 1.00 | 48.57 | C |
| ATOM | 13776 | O | MET | G | 119 | 4.384 | 23.007 | 94.316 | 1.00 | 49.87 | O |
| ATOM | 13777 | N | ASP | G | 120 | 6.292 | 23.206 | 95.554 | 1.00 | 54.58 | N |
| ATOM | 13779 | CA | ASP | G | 120 | 5.901 | 22.523 | 96.806 | 1.00 | 53.35 | C |
| ATOM | 13781 | CB | ASP | G | 120 | 7.167 | 22.310 | 97.665 | 1.00 | 53.59 | C |
| ATOM | 13784 | CG | ASP | G | 120 | 6.920 | 21.489 | 98.920 | 1.00 | 63.19 | C |
| ATOM | 13785 | OD1 | ASP | G | 120 | 5.728 | 21.248 | 99.230 | 1.00 | 60.75 | O |
| ATOM | 13786 | OD2 | ASP | G | 120 | 7.859 | 21.039 | 99.633 | 1.00 | 63.54 | O |
| ATOM | 13787 | C | ASP | G | 120 | 4.959 | 23.358 | 97.653 | 1.00 | 51.51 | C |
| ATOM | 13788 | O | ASP | G | 120 | 5.436 | 24.184 | 98.407 | 1.00 | 45.00 | O |
| ATOM | 13789 | N | LEU | G | 121 | 3.649 | 23.131 | 97.606 | 1.00 | 56.38 | N |
| ATOM | 13791 | CA | LEU | G | 121 | 2.809 | 23.849 | 98.556 | 1.00 | 54.60 | C |
| ATOM | 13793 | CB | LEU | G | 121 | 1.950 | 24.875 | 97.862 | 1.00 | 47.74 | C |
| ATOM | 13796 | CG | LEU | G | 121 | 2.812 | 25.625 | 96.866 | 1.00 | 43.43 | C |
| ATOM | 13798 | CD1 | LEU | G | 121 | 1.868 | 26.042 | 95.785 | 1.00 | 52.22 | C |
| ATOM | 13802 | CD2 | LEU | G | 121 | 3.443 | 26.838 | 97.568 | 1.00 | 69.85 | C |
| ATOM | 13806 | C | LEU | G | 121 | 1.947 | 23.011 | 99.460 | 1.00 | 58.45 | C |
| ATOM | 13807 | O | LEU | G | 121 | 1.787 | 21.812 | 99.276 | 1.00 | 59.00 | O |
| ATOM | 13808 | N | ASP | G | 122 | 1.424 | 23.688 | 100.468 | 1.00 | 65.37 | N |
| ATOM | 13810 | CA | ASP | G | 122 | 0.569 | 23.063 | 101.463 | 1.00 | 70.05 | C |
| ATOM | 13812 | CB | ASP | G | 122 | 1.402 | 22.398 | 102.566 | 1.00 | 75.28 | C |
| ATOM | 13815 | CG | ASP | G | 122 | 0.647 | 21.282 | 103.301 | 1.00 | 74.68 | C |
| ATOM | 13816 | OD1 | ASP | G | 122 | −0.268 | 20.660 | 102.696 | 1.00 | 60.46 | O |
| ATOM | 13817 | OD2 | ASP | G | 122 | 0.946 | 20.965 | 104.482 | 1.00 | 63.01 | O |
| ATOM | 13818 | C | ASP | G | 122 | −0.355 | 24.135 | 102.032 | 1.00 | 65.63 | C |
| ATOM | 13819 | O | ASP | G | 122 | −0.204 | 24.584 | 103.173 | 1.00 | 63.15 | O |
| ATOM | 13820 | N | VAL | G | 123 | −1.303 | 24.513 | 101.183 | 1.00 | 64.19 | N |
| ATOM | 13822 | CA | VAL | G | 123 | −2.331 | 25.501 | 101.471 | 1.00 | 66.99 | C |
| ATOM | 13824 | CB | VAL | G | 123 | −2.838 | 26.032 | 100.133 | 1.00 | 65.35 | C |
| ATOM | 13826 | CG1 | VAX | G | 123 | −3.105 | 27.524 | 100.182 | 1.00 | 58.77 | C |
| ATOM | 13830 | CG2 | VAL | G | 123 | −1.797 | 25.691 | 99.073 | 1.00 | 72.59 | C |
| ATOM | 13834 | C | VAL | G | 123 | −3.480 | 24.827 | 102.220 | 1.00 | 66.84 | C |
| ATOM | 13835 | O | VAL | G | 123 | −3.971 | 23.792 | 101.785 | 1.00 | 64.12 | O |
| ATOM | 13836 | N | GLU | G | 124 | −3.880 | 25.377 | 103.360 | 1.00 | 68.38 | N |
| ATOM | 13838 | CA | GLU | G | 124 | −5.132 | 24.967 | 103.984 | 1.00 | 73.34 | C |
| ATOM | 13840 | CB | GLU | G | 124 | −5.199 | 25.498 | 105.415 | 1.00 | 73.64 | C |
| ATOM | 13843 | CG | GLU | G | 124 | −5.515 | 24.444 | 106.452 | 1.00 | 80.72 | C |
| ATOM | 13846 | CD | GLU | G | 124 | −5.179 | 24.910 | 107.852 | 1.00 | 79.05 | C |
| ATOM | 13847 | OE1 | GLU | G | 124 | −5.329 | 26.106 | 108.139 | 1.00 | 75.55 | O |
| ATOM | 13848 | OE2 | GLU | G | 124 | −4.772 | 24.074 | 108.672 | 1.00 | 96.92 | O |
| ATOM | 13849 | C | GLU | G | 124 | −6.303 | 25.529 | 103.180 | 1.00 | 72.33 | C |
| ATOM | 13850 | O | GLU | G | 124 | −6.326 | 26.740 | 102.924 | 1.00 | 68.21 | O |
| ATOM | 13851 | N | GLN | G | 125 | −7.254 | 24.660 | 102.813 | 1.00 | 69.43 | N |
| ATOM | 13853 | CA | GLN | G | 125 | −8.557 | 25.069 | 102.248 | 1.00 | 67.82 | C |
| ATOM | 13855 | CB | GLN | G | 125 | −9.408 | 23.840 | 101.883 | 1.00 | 63.24 | C |
| ATOM | 13858 | CG | GLN | G | 125 | −9.210 | 23.296 | 100.485 | 1.00 | 71.53 | C |
| ATOM | 13861 | CD | GLN | G | 125 | −9.921 | 24.077 | 99.389 | 1.00 | 74.28 | C |
| ATOM | 13862 | OE1 | GLN | G | 125 | −9.296 | 24.431 | 98.386 | 1.00 | 80.63 | O |
| ATOM | 13863 | NE2 | GLN | G | 125 | −11.234 | 24.261 | 99.530 | 1.00 | 68.42 | N |
| ATOM | 13866 | C | GLN | G | 125 | −9.363 | 25.917 | 103.247 | 1.00 | 63.44 | C |
| ATOM | 13867 | O | GLN | G | 125 | −9.502 | 25.523 | 104.410 | 1.00 | 63.41 | O |
| ATOM | 13868 | N | LEU | G | 126 | −9.894 | 27.058 | 102.811 | 1.00 | 51.08 | N |
| ATOM | 13870 | CA | LEU | G | 126 | −10.849 | 27.791 | 103.631 | 1.00 | 53.30 | C |
| ATOM | 13872 | CB | LEU | G | 126 | −10.707 | 29.317 | 103.519 | 1.00 | 52.49 | C |
| ATOM | 13875 | CG | LEU | G | 126 | −9.305 | 29.907 | 103.672 | 1.00 | 55.14 | C |
| ATOM | 13877 | CD1 | LEU | G | 126 | −9.373 | 31.421 | 103.714 | 1.00 | 63.42 | C |
| ATOM | 13881 | CD2 | LEU | G | 126 | −8.622 | 29.363 | 104.916 | 1.00 | 73.53 | C |
| ATOM | 13885 | C | LEU | G | 126 | −12.248 | 27.397 | 103.200 | 1.00 | 57.02 | C |
| ATOM | 13886 | O | LEU | G | 126 | −12.560 | 27.372 | 102.013 | 1.00 | 55.28 | O |
| ATOM | 13887 | N | GLY | G | 127 | −13.094 | 27.098 | 104.179 | 1.00 | 57.74 | N |
| ATOM | 13889 | CA | GLY | G | 127 | −14.490 | 26.815 | 103.905 | 1.00 | 56.40 | C |
| ATOM | 13892 | C | GLY | G | 127 | −15.234 | 28.122 | 103.773 | 1.00 | 56.99 | C |
| ATOM | 13893 | O | GLY | G | 127 | −15.043 | 29.035 | 104.581 | 1.00 | 56.57 | O |
| ATOM | 13894 | N | ILE | G | 128 | −16.023 | 28.221 | 102.707 | 1.00 | 60.32 | N |
| ATOM | 13896 | CA | ILE | G | 128 | −16.816 | 29.406 | 102.409 | 1.00 | 56.57 | C |
| ATOM | 13898 | CB | ILE | G | 128 | −16.914 | 29.640 | 100.895 | 1.00 | 55.15 | C |
| ATOM | 13900 | CG1 | ILE | G | 128 | −15.651 | 30.362 | 100.399 | 1.00 | 63.01 | C |
| ATOM | 13903 | CD1 | ILE | G | 128 | −14.953 | 29.751 | 99.226 | 1.00 | 61.15 | C |
| ATOM | 13907 | CG2 | ILE | G | 128 | −18.162 | 30.496 | 100.611 | 1.00 | 64.45 | C |
| ATOM | 13911 | C | ILE | G | 128 | −18.188 | 29.064 | 102.891 | 1.00 | 55.21 | C |
| ATOM | 13912 | O | ILE | G | 128 | −18.843 | 28.211 | 102.327 | 1.00 | 51.17 | O |
| ATOM | 13913 | N | PRO | G | 129 | −18.630 | 29.668 | 103.972 | 1.00 | 63.62 | N |
| ATOM | 13914 | CA | PRO | G | 129 | −19.935 | 29.280 | 104.511 | 1.00 | 62.97 | C |
| ATOM | 13916 | CB | PRO | G | 129 | −20.055 | 30.116 | 105.794 | 1.00 | 67.72 | C |
| ATOM | 13919 | CG | PRO | G | 129 | −18.634 | 30.591 | 106.087 | 1.00 | 69.50 | C |
| ATOM | 13922 | CD | PRO | G | 129 | −17.993 | 30.765 | 104.720 | 1.00 | 65.89 | C |
| ATOM | 13925 | C | PRO | G | 129 | −21.003 | 29.627 | 103.468 | 1.00 | 63.75 | C |
| ATOM | 13926 | O | PRO | G | 129 | −20.746 | 30.502 | 102.604 | 1.00 | 60.10 | O |
| ATOM | 13927 | N | GLU | G | 130 | −22.134 | 28.917 | 103.530 | 1.00 | 61.87 | N |
| ATOM | 13929 | CA | GLU | G | 130 | −23.324 | 29.238 | 102.737 | 1.00 | 65.49 | C |
| ATOM | 13931 | CB | GLU | G | 130 | −24.336 | 28.061 | 102.751 | 1.00 | 65.00 | C |
| ATOM | 13934 | CG | GLU | G | 130 | −24.528 | 27.336 | 101.405 | 1.00 | 81.50 | C |
| ATOM | 13937 | CD | GLU | G | 130 | −24.935 | 28.268 | 100.243 | 1.00 | 104.02 | C |
| ATOM | 13938 | OE1 | GLU | G | 130 | −24.109 | 29.058 | 99.713 | 1.00 | 91.76 | O |
| ATOM | 13939 | OE2 | GLU | G | 130 | −26.118 | 28.247 | 99.831 | 1.00 | 110.08 | O |
| ATOM | 13940 | C | GLU | G | 130 | −23.916 | 30.591 | 103.200 | 1.00 | 63.59 | C |
| ATOM | 13941 | O | GLU | G | 130 | −23.963 | 30.882 | 104.399 | 1.00 | 70.39 | O |
| ATOM | 13942 | N | GLN | G | 131 | −24.248 | 31.459 | 102.247 | 1.00 | 62.18 | N |
| ATOM | 13944 | CA | GLN | G | 131 | −24.571 | 32.861 | 102.516 | 1.00 | 64.30 | C |
| ATOM | 13946 | CB | GLN | G | 131 | −23.429 | 33.810 | 102.078 | 1.00 | 67.65 | C |
| ATOM | 13949 | CG | GLN | G | 131 | −22.424 | 34.302 | 103.151 | 1.00 | 75.51 | C |
| ATOM | 13952 | CD | GLN | G | 131 | −23.044 | 35.250 | 104.162 | 1.00 | 78.14 | C |
| ATOM | 13953 | OE1 | GLN | G | 131 | −23.962 | 36.009 | 103.821 | 1.00 | 56.79 | O |
| ATOM | 13954 | NE2 | GLN | G | 131 | −22.573 | 35.183 | 105.413 | 1.00 | 67.33 | N |
| ATOM | 13957 | C | GLN | G | 131 | −25.784 | 33.229 | 101.676 | 1.00 | 64.27 | C |
| ATOM | 13958 | O | GLN | G | 131 | −25.850 | 32.908 | 100.488 | 1.00 | 58.50 | O |
| ATOM | 13959 | N | GLU | G | 132 | −26.712 | 33.971 | 102.270 | 1.00 | 73.17 | N |
| ATOM | 13961 | CA | GLU | G | 132 | −27.733 | 34.665 | 101.484 | 1.00 | 76.56 | C |
| ATOM | 13963 | CB | GLU | G | 132 | −29.133 | 34.448 | 102.122 | 1.00 | 83.21 | C |
| ATOM | 13966 | CG | GLU | G | 132 | −30.182 | 33.830 | 101.184 | 1.00 | 80.75 | C |
| ATOM | 13969 | CD | GLU | G | 132 | −30.579 | 34.786 | 100.054 | 1.00 | 107.73 | C |
| ATOM | 13970 | OE1 | GLU | G | 132 | −29.724 | 35.071 | 99.177 | 1.00 | 105.58 | O |
| ATOM | 13971 | OE2 | GLU | G | 132 | −31.727 | 35.302 | 100.048 | 1.00 | 114.41 | O |
| ATOM | 13972 | C | GLU | G | 132 | −27.330 | 36.148 | 101.297 | 1.00 | 68.31 | C |
| ATOM | 13973 | O | GLU | G | 132 | −26.727 | 36.745 | 102.200 | 1.00 | 59.04 | O |
| ATOM | 13974 | N | TYR | G | 133 | −27.555 | 36.707 | 100.105 | 1.00 | 58.51 | N |
| ATOM | 13976 | CA | TYR | G | 133 | −27.049 | 38.050 | 99.818 | 1.00 | 55.78 | C |
| ATOM | 13978 | CB | TYR | G | 133 | −26.092 | 38.129 | 98.595 | 1.00 | 60.67 | C |
| ATOM | 13981 | CG | TYR | G | 133 | −24.750 | 37.459 | 98.797 | 1.00 | 53.11 | C |
| ATOM | 13982 | CD1 | TYR | G | 133 | −24.596 | 36.109 | 98.553 | 1.00 | 58.79 | C |
| ATOM | 13984 | CE1 | TYR | G | 133 | −23.395 | 35.463 | 98.799 | 1.00 | 55.40 | C |
| ATOM | 13986 | CZ | TYR | G | 133 | −22.322 | 36.151 | 99.298 | 1.00 | 43.94 | C |
| ATOM | 13987 | OH | TYR | G | 133 | −21.148 | 35.474 | 99.558 | 1.00 | 52.84 | O |
| ATOM | 13989 | CE2 | TYR | G | 133 | −22.451 | 37.490 | 99.580 | 1.00 | 48.56 | C |
| ATOM | 13991 | CD2 | TYR | G | 133 | −23.669 | 38.138 | 99.337 | 1.00 | 55.64 | C |
| ATOM | 13993 | C | TYR | G | 133 | −28.174 | 39.082 | 99.687 | 1.00 | 53.99 | C |
| ATOM | 13994 | O | TYR | G | 133 | −29.114 | 38.968 | 98.881 | 1.00 | 44.38 | O |
| ATOM | 13995 | N | SER | G | 134 | −28.013 | 40.141 | 100.474 | 1.00 | 51.11 | N |
| ATOM | 13997 | CA | SER | G | 134 | −29.013 | 41.179 | 100.558 | 1.00 | 49.41 | C |
| ATOM | 13999 | CB | SER | G | 134 | −28.564 | 42.274 | 101.513 | 1.00 | 53.30 | C |
| ATOM | 14002 | OG | SER | G | 134 | −28.383 | 41.782 | 102.835 | 1.00 | 45.70 | O |
| ATOM | 14004 | C | SER | G | 134 | −29.296 | 41.725 | 99.176 | 1.00 | 45.46 | C |
| ATOM | 14005 | O | SER | G | 134 | −30.419 | 41.931 | 98.786 | 1.00 | 50.74 | O |
| ATOM | 14006 | N | CYS | G | 135 | −28.285 | 41.928 | 98.369 | 1.00 | 55.14 | N |
| ATOM | 14008 | CA | CYS | G | 135 | −28.617 | 42.539 | 97.108 | 1.00 | 62.88 | C |
| ATOM | 14010 | CB | CYS | G | 135 | −28.692 | 44.071 | 97.264 | 1.00 | 55.40 | C |
| ATOM | 14013 | SG | CYS | G | 135 | −27.611 | 44.922 | 96.140 | 1.00 | 102.35 | S |
| ATOM | 14014 | C | CYS | G | 135 | −27.643 | 41.950 | 96.100 | 1.00 | 53.67 | C |
| ATOM | 14015 | O | CYS | G | 135 | −26.562 | 41.590 | 96.510 | 1.00 | 54.11 | O |
| ATOM | 14016 | N | VAL | G | 136 | −28.115 | 41.607 | 94.896 | 1.00 | 55.19 | N |
| ATOM | 14018 | CA | VAL | G | 136 | −27.259 | 41.041 | 93.844 | 1.00 | 52.11 | C |
| ATOM | 14020 | CB | VAL | G | 136 | −27.563 | 39.548 | 93.548 | 1.00 | 47.25 | C |
| ATOM | 14022 | CG1 | VAL | G | 136 | −26.556 | 38.949 | 92.605 | 1.00 | 50.36 | C |
| ATOM | 14026 | CG2 | VAL | G | 136 | −27.486 | 38.724 | 94.801 | 1.00 | 57.46 | C |
| ATOM | 14030 | C | VAL | G | 136 | −27.435 | 41.846 | 92.572 | 1.00 | 47.12 | C |
| ATOM | 14031 | O | VAL | G | 136 | −28.479 | 41.749 | 91.960 | 1.00 | 52.08 | O |
| ATOM | 14032 | N | VAL | G | 137 | −26.446 | 42.666 | 92.212 | 1.00 | 46.10 | N |
| ATOM | 14034 | CA | VAL | G | 137 | −26.469 | 43.395 | 90.947 | 1.00 | 46.84 | C |
| ATOM | 14036 | CB | VAL | G | 137 | −25.577 | 44.629 | 90.961 | 1.00 | 51.41 | C. |
| ATOM | 14038 | CG1 | VAL | G | 137 | −25.909 | 45.505 | 89.725 | 1.00 | 51.10 | C |
| ATOM | 14042 | CG2 | VAL | G | 137 | −25.703 | 45.393 | 92.290 | 1.00 | 60.44 | C |
| ATOM | 14046 | C | VAL | G | 137 | −25.883 | 42.587 | 89.809 | 1.00 | 44.25 | C |
| ATOM | 14047 | O | VAL | G | 137 | −24.858 | 41.931 | 89.966 | 1.00 | 50.05 | O |
| ATOM | 14048 | N | LYS | G | 138 | −26.477 | 42.702 | 88.632 | 1.00 | 38.49 | N |
| ATOM | 14050 | CA | LYS | G | 138 | −25.946 | 41.996 | 87.476 | 1.00 | 42.98 | C |
| ATOM | 14052 | CB | LYS | G | 138 | −26.962 | 41.028 | 86.876 | 1.00 | 43.46 | C |
| ATOM | 14055 | CG | LYS | G | 138 | −26.368 | 39.722 | 86.348 | 1.00 | 62.65 | C |
| ATOM | 14058 | CD | LYS | G | 138 | −27.227 | 39.013 | 85.267 | 1.00 | 80.28 | C |
| ATOM | 14061 | CE | LYS | G | 138 | −26.684 | 39.188 | 83.842 | 1.00 | 83.49 | C |
| ATOM | 14064 | NZ | LYS | G | 138 | −26.585 | 37.877 | 83.133 | 1.00 | 83.79 | N |
| ATOM | 14068 | C | LYS | G | 138 | −25.755 | 43.087 | 86.501 | 1.00 | 35.63 | C |
| ATOM | 14069 | O | LYS | G | 138 | −26.714 | 43.789 | 86.258 | 1.00 | 37.75 | O |
| ATOM | 14070 | N | MET | G | 139 | −24.537 | 43.263 | 85.995 | 1.00 | 44.25 | N |
| ATOM | 14072 | CA | MET | G | 139 | −24.271 | 44.349 | 85.036 | 1.00 | 49.17 | C |
| ATOM | 14074 | CB | MET | G | 139 | −23.860 | 45.632 | 85.746 | 1.00 | 52.59 | C |
| ATOM | 14077 | CG | MET | G | 139 | −22.562 | 45.530 | 86.506 | 1.00 | 54.69 | C |
| ATOM | 14080 | SD | MET | G | 139 | −22.166 | 47.167 | 87.154 | 1.00 | 62.06 | S |
| ATOM | 14081 | CE | MET | G | 139 | −21.470 | 46.494 | 88.543 | 1.00 | 36.83 | C |
| ATOM | 14085 | C | MET | G | 139 | −23.202 | 44.040 | 84.017 | 1.00 | 45.96 | C |
| ATOM | 14086 | O | MET | G | 139 | −22.569 | 42.978 | 84.056 | 1.00 | 41.22 | O |
| ATOM | 14087 | N | PRO | G | 140 | −23.060 | 44.945 | 83.060 | 1.00 | 39.61 | N |
| ATOM | 14088 | CA | PRO | G | 140 | −22.084 | 44.761 | 81.985 | 1.00 | 40.88 | C |
| ATOM | 14090 | CB | PRO | G | 140 | −22.379 | 45.888 | 81.012 | 1.00 | 35.35 | C |
| ATOM | 14093 | CG | PRO | G | 140 | −23.701 | 46.394 | 81.385 | 1.00 | 42.94 | C |
| ATOM | 14096 | CD | PRO | G | 140 | −23.931 | 46.103 | 82.847 | 1.00 | 40.73 | C |
| ATOM | 14099 | C | PRO | G | 140 | −20.690 | 44.923 | 82.540 | 1.00 | 49.28 | C |
| ATOM | 14100 | O | PRO | G | 140 | −20.441 | 45.971 | 83.107 | 1.00 | 53.11 | O |
| ATOM | 14101 | N | SER | G | 141 | −19.838 | 43.906 | 82.401 | 1.00 | 53.62 | N |
| ATOM | 14103 | CA | SER | G | 141 | −18.528 | 43.847 | 83.055 | 1.00 | 50.16 | C |
| ATOM | 14105 | CB | SER | G | 141 | −17.816 | 42.524 | 82.717 | 1.00 | 53.07 | C |
| ATOM | 14108 | OG | SER | G | 141 | −17.501 | 42.444 | 81.341 | 1.00 | 36.61 | O |
| ATOM | 14110 | C | SER | G | 141 | −17.600 | 44.990 | 82.668 | 1.00 | 40.38 | C |
| ATOM | 14111 | O | SER | G | 141 | −16.824 | 45.488 | 83.473 | 1.00 | 40.26 | O |
| ATOM | 14112 | N | GLY | G | 142 | −17.640 | 45.393 | 81.415 | 1.00 | 34.65 | N |
| ATOM | 14114 | CA | GLY | G | 142 | −16.849 | 46.538 | 81.015 | 1.00 | 40.12 | C |
| ATOM | 14117 | C | GLY | G | 142 | −17.208 | 47.791 | 81.779 | 1.00 | 42.06 | C |
| ATOM | 14118 | O | GLY | G | 142 | −16.341 | 48.575 | 82.134 | 1.00 | 53.11 | O |
| ATOM | 14119 | N | GLU | G | 143 | −18.496 | 47.999 | 81.997 | 1.00 | 46.05 | N |
| ATOM | 14121 | CA | GLU | G | 143 | −18.977 | 49.157 | 82.741 | 1.00 | 45.39 | C |
| ATOM | 14123 | CB | GLU | G | 143 | −20.508 | 49.117 | 82.756 | 1.00 | 53.20 | C |
| ATOM | 14126 | CG | GLU | G | 143 | −21.170 | 50.363 | 83.286 | 1.00 | 59.39 | C |
| ATOM | 14129 | CD | GLU | G | 143 | −20.795 | 51.561 | 82.465 | 1.00 | 65.78 | C |
| ATOM | 14130 | OE1 | GLU | G | 143 | −19.908 | 52.296 | 82.939 | 1.00 | 79.57 | O |
| ATOM | 14131 | OE2 | GLU | G | 143 | −21.348 | 51.717 | 81.352 | 1.00 | 61.64 | O |
| ATOM | 14132 | C | GLU | G | 143 | −18.435 | 49.099 | 84.165 | 1.00 | 37.87 | C |
| ATOM | 14133 | O | GLU | G | 143 | −18.014 | 50.100 | 84.721 | 1.00 | 42.51 | O |
| ATOM | 14134 | N | PHE | G | 144 | −18.452 | 47.929 | 84.782 | 1.00 | 31.66 | N |
| ATOM | 14136 | CA | PHE | G | 144 | −17.967 | 47.828 | 86.153 | 1.00 | 39.82 | C |
| ATOM | 14138 | CB | PHE | G | 144 | −18.266 | 46.454 | 86.753 | 1.00 | 36.46 | C |
| ATOM | 14141 | CG | PHE | G | 144 | −17.741 | 46.271 | 88.155 | 1.00 | 30.79 | C |
| ATOM | 14142 | CD1 | PHE | G | 144 | −18.170 | 47.090 | 89.182 | 1.00 | 35.16 | C |
| ATOM | 14144 | CE1 | PHE | G | 144 | −17.698 | 46.910 | 90.471 | 1.00 | 45.54 | C |
| ATOM | 14146 | CZ | PHE | G | 144 | −16.747 | 45.931 | 90.725 | 1.00 | 44.87 | C |
| ATOM | 14148 | CE2 | PHE | G | 144 | −16.301 | 45.125 | 89.712 | 1.00 | 32.20 | C |
| ATOM | 14150 | CD2 | PHE | G | 144 | −16.814 | 45.280 | 88.445 | 1.00 | 44.73 | C |
| ATOM | 14152 | C | PHE | G | 144 | −16.470 | 48.037 | 86.166 | 1.00 | 39.74 | C |
| ATOM | 14153 | O | PHE | G | 144 | −15.927 | 48.630 | 87.086 | 1.00 | 40.79 | O |
| ATOM | 14154 | N | ALA | G | 145 | −15.807 | 47.483 | 85.163 | 1.00 | 43.22 | N |
| ATOM | 14156 | CA | ALA | G | 145 | −14.396 | 47.757 | 84.971 | 1.00 | 46.68 | C |
| ATOM | 14158 | CB | ALA | G | 145 | −13.838 | 46.938 | 83.786 | 1.00 | 38.57 | C |
| ATOM | 14162 | C | ALA | G | 145 | −14.175 | 49.274 | 84.809 | 1.00 | 48.95 | C |
| ATOM | 14163 | O | ALA | G | 145 | −13.219 | 49.796 | 85.386 | 1.00 | 48.46 | O |
| ATOM | 14164 | N | ARG | G | 146 | −15.064 | 49.992 | 84.107 | 1.00 | 45.80 | N |
| ATOM | 14166 | CA | ARG | G | 146 | −14.802 | 51.401 | 83.747 | 1.00 | 47.91 | C |
| ATOM | 14168 | CB | ARG | G | 146 | −15.732 | 51.907 | 82.636 | 1.00 | 48.55 | C |
| ATOM | 14171 | CG | ARG | G | 146 | −15.326 | 53.241 | 81.992 | 1.00 | 62.02 | C |
| ATOM | 14174 | CD | ARG | G | 146 | −15.841 | 53.491 | 80.531 | 1.00 | 82.35 | C |
| ATOM | 14177 | NE | ARG | G | 146 | −14.817 | 53.309 | 79.473 | 1.00 | 109.15 | N |
| ATOM | 14179 | CZ | ARG | G | 146 | −13.975 | 54.244 | 78.984 | 1.00 | 104.66 | C |
| ATOM | 14180 | NH1 | ARG | G | 146 | −13.984 | 55.494 | 79.440 | 1.00 | 111.56 | N |
| ATOM | 14183 | NH2 | ARG | G | 146 | −13.094 | 53.926 | 78.035 | 1.00 | 70.54 | N |
| ATOM | 14186 | C | ARG | G | 146 | −14.961 | 52.267 | 84.986 | 1.00 | 46.96 | C |
| ATOM | 14187 | O | ARG | G | 146 | −14.279 | 53.265 | 85.163 | 1.00 | 55.31 | O |
| ATOM | 14188 | N | ILE | G | 147 | −15.848 | 51.840 | 85.868 | 1.00 | 41.46 | N |
| ATOM | 14190 | CA | ILE | G | 147 | −16.280 | 52.658 | 86.965 | 1.00 | 38.87 | C |
| ATOM | 14192 | CB | ILE | G | 147 | −17.627 | 52.129 | 87.440 | 1.00 | 45.31 | C |
| ATOM | 14194 | CG1 | ILE | G | 147 | −18.742 | 52.813 | 86.640 | 1.00 | 45.62 | C |
| ATOM | 14197 | CD1 | ILE | G | 147 | −20.127 | 52.165 | 86.828 | 1.00 | 42.26 | C |
| ATOM | 14201 | CG2 | ILE | G | 147 | −17.746 | 52.247 | 88.969 | 1.00 | 41.74 | C |
| ATOM | 14205 | C | ILE | G | 147 | −15.232 | 52.530 | 88.059 | 1.00 | 39.25 | C |
| ATOM | 14206 | O | ILE | G | 147 | −14.787 | 53.523 | 88.606 | 1.00 | 44.64 | O |
| ATOM | 14207 | N | CYS | G | 148 | −14.823 | 51.309 | 88.370 | 1.00 | 45.08 | N |
| ATOM | 14209 | CA | CYS | G | 148 | −13.706 | 51.086 | 89.273 | 1.00 | 47.77 | C |
| ATOM | 14211 | CB | CYS | G | 148 | −13.386 | 49.599 | 89.363 | 1.00 | 53.70 | C |
| ATOM | 14214 | SG | CYS | G | 148 | −14.626 | 48.510 | 90.118 | 1.00 | 60.45 | S |
| ATOM | 14215 | C | CYS | G | 148 | −12.449 | 51.811 | 88.819 | 1.00 | 49.89 | C |
| ATOM | 14216 | O | CYS | G | 148 | −11.651 | 52.201 | 89.636 | 1.00 | 57.36 | O |
| ATOM | 14217 | N | ARG | G | 149 | −12.240 | 51.977 | 87.524 | 1.00 | 56.88 | N |
| ATOM | 14219 | CA | ARG | G | 149 | −11.058 | 52.694 | 87.049 | 1.00 | 57.90 | C |
| ATOM | 14221 | CB | ARG | G | 149 | −10.846 | 52.427 | 85.554 | 1.00 | 56.16 | C |
| ATOM | 14224 | CG | ARG | G | 149 | −9.497 | 52.846 | 85.035 | 1.00 | 70.41 | C |
| ATOM | 14227 | CD | ARG | G | 149 | −9.290 | 52.687 | 83.532 | 1.00 | 95.35 | C |
| ATOM | 14230 | NE | ARG | G | 149 | −10.135 | 53.571 | 82.727 | 1.00 | 100.92 | N |
| ATOM | 14232 | CZ | ARG | G | 149 | −11.098 | 53.139 | 81.914 | 1.00 | 109.47 | C |
| ATOM | 14233 | NH1 | ARG | G | 149 | −11.378 | 51.839 | 81.811 | 1.00 | 107.27 | N |
| ATOM | 14236 | NH2 | ARG | G | 149 | −11.809 | 54.010 | 81.213 | 1.00 | 98.25 | N |
| ATOM | 14239 | C | ARG | G | 149 | −11.224 | 54.204 | 87.270 | 1.00 | 57.09 | C |
| ATOM | 14240 | O | ARG | G | 149 | −10.342 | 54.871 | 87.779 | 1.00 | 47.66 | O |
| ATOM | 14241 | N | ASP | G | 150 | −12.340 | 54.774 | 86.850 | 1.00 | 50.70 | N |
| ATOM | 14243 | CA | ASP | G | 150 | −12.528 | 56.205 | 87.032 | 1.00 | 54.91 | C |
| ATOM | 14245 | CB | ASP | G | 150 | −13.826 | 56.583 | 86.336 | 1.00 | 56.28 | C |
| ATOM | 14248 | CG | ASP | G | 150 | −13.726 | 56.456 | 84.852 | 1.00 | 49.05 | C |
| ATOM | 14249 | OD1 | ASP | G | 150 | −12.591 | 56.359 | 84.344 | 1.00 | 73.35 | O |
| ATOM | 14250 | OD2 | ASP | G | 150 | −14.718 | 56.523 | 84.119 | 1.00 | 55.65 | O |
| ATOM | 14251 | C | ASP | G | 150 | −12.563 | 56.698 | 88.499 | 1.00 | 57.39 | C |
| ATOM | 14252 | O | ASP | G | 150 | −11.905 | 57.661 | 88.856 | 1.00 | 54.26 | O |
| ATOM | 14253 | N | LEU | G | 151 | −13.361 | 56.076 | 89.356 | 1.00 | 51.50 | N |
| ATOM | 14255 | CA | LEU | G | 151 | −13.339 | 56.450 | 90.746 | 1.00 | 45.86 | C |
| ATOM | 14257 | CB | LEU | G | 151 | −14.318 | 55.590 | 91.529 | 1.00 | 39.02 | C |
| ATOM | 14260 | CG | LEU | G | 151 | −15.776 | 55.773 | 91.145 | 1.00 | 50.71 | C |
| ATOM | 14262 | CD1 | LEU | G | 151 | −16.648 | 55.077 | 92.171 | 1.00 | 51.42 | C |
| ATOM | 14266 | CD2 | LEU | G | 151 | −16.190 | 57.221 | 90.976 | 1.00 | 41.58 | C |
| ATOM | 14270 | C | LEU | G | 151 | −11.947 | 56.332 | 91.374 | 1.00 | 49.16 | C |
| ATOM | 14271 | O | LEU | G | 151 | −11.602 | 57.044 | 92.321 | 1.00 | 48.24 | O |
| ATOM | 14272 | N | SER | G | 152 | −11.128 | 55.398 | 90.925 | 1.00 | 52.51 | N |
| ATOM | 14274 | CA | SER | G | 152 | −9.798 | 55.282 | 91.548 | 1.00 | 50.36 | C |
| ATOM | 14276 | CB | SER | G | 152 | −9.143 | 53.974 | 91.121 | 1.00 | 35.75 | C |
| ATOM | 14279 | OG | SER | G | 152 | −8.776 | 54.174 | 89.784 | 1.00 | 33.51 | O |
| ATOM | 14281 | C | SER | G | 152 | −8.878 | 56.477 | 91.186 | 1.00 | 49.89 | C |
| ATOM | 14282 | O | SER | G | 152 | −7.863 | 56.686 | 91.842 | 1.00 | 56.19 | O |
| ATOM | 14283 | N | HIS | G | 153 | −9.274 | 57.276 | 90.194 | 1.00 | 45.99 | N |
| ATOM | 14285 | CA | HIS | G | 153 | −8.793 | 58.645 | 89.992 | 1.00 | 49.90 | C |
| ATOM | 14287 | CB | HIS | G | 153 | −9.262 | 59.159 | 88.626 | 1.00 | 53.09 | C |
| ATOM | 14290 | CG | HIS | G | 153 | −8.658 | 58.424 | 87.469 | 1.00 | 72.01 | C |
| ATOM | 14291 | ND1 | HIS | G | 153 | −8.355 | 59.033 | 86.272 | 1.00 | 89.87 | N |
| ATOM | 14293 | CE1 | HIS | G | 153 | −7.821 | 58.143 | 85.453 | 1.00 | 111.06 | C |
| ATOM | 14295 | NE2 | HIS | G | 153 | −7.778 | 56.976 | 86.071 | 1.00 | 88.26 | N |
| ATOM | 14297 | CD2 | HIS | G | 153 | −8.300 | 57.125 | 87.331 | 1.00 | 86.26 | C |
| ATOM | 14299 | C | HIS | G | 153 | −9.234 | 59.651 | 91.052 | 1.00 | 47.43 | C |
| ATOM | 14300 | O | HIS | G | 153 | −8.718 | 60.754 | 91.121 | 1.00 | 50.41 | O |
| ATOM | 14301 | N | ILE | G | 154 | −10.193 | 59.278 | 91.887 | 1.00 | 53.23 | N |
| ATOM | 14303 | CA | ILE | G | 154 | −10.854 | 60.239 | 92.771 | 1.00 | 50.06 | C |
| ATOM | 14305 | CB | ILE | G | 154 | −12.367 | 60.144 | 92.615 | 1.00 | 43.44 | C |
| ATOM | 14307 | CG1 | ILE | G | 154 | −12.747 | 60.375 | 91.151 | 1.00 | 47.33 | C |
| ATOM | 14310 | CD1 | ILE | G | 154 | −12.427 | 61.768 | 90.639 | 1.00 | 55.58 | C |
| ATOM | 14314 | CG2 | ILE | G | 154 | −13.009 | 61.107 | 93.571 | 1.00 | 44.97 | C |
| ATOM | 14318 | C | ILE | G | 154 | −10.515 | 59.990 | 94.235 | 1.00 | 45.64 | C |
| ATOM | 14319 | O | ILE | G | 154 | −10.077 | 60.891 | 94.938 | 1.00 | 51.64 | O |
| ATOM | 14320 | N | GLY | G | 155 | −10.749 | 58.763 | 94.677 | 1.00 | 38.52 | N |
| ATOM | 14322 | CA | GLY | G | 155 | −10.082 | 58.203 | 95.822 | 1.00 | 37.98 | C |
| ATOM | 14325 | C | GLY | G | 155 | −9.807 | 56.709 | 95.760 | 1.00 | 41.70 | C |
| ATOM | 14326 | O | GLY | G | 155 | −9.592 | 56.069 | 94.732 | 1.00 | 54.43 | O |
| ATOM | 14327 | N | ASP | G | 156 | −9.807 | 56.130 | 96.935 | 1.00 | 39.79 | N |
| ATOM | 14329 | CA | ASP | G | 156 | −9.047 | 54.943 | 97.202 | 1.00 | 43.67 | C |
| ATOM | 14331 | CB | ASP | G | 156 | −8.359 | 55.156 | 98.526 | 1.00 | 49.33 | C |
| ATOM | 14334 | CG | ASP | G | 156 | −6.897 | 55.283 | 98.366 | 1.00 | 63.19 | C |
| ATOM | 14335 | OD1 | ASP | G | 156 | −6.205 | 55.002 | 99.361 | 1.00 | 93.08 | O |
| ATOM | 14336 | OD2 | ASP | G | 156 | −6.372 | 55.662 | 97.295 | 1.00 | 81.42 | O |
| ATOM | 14337 | C | ASP | G | 156 | −9.971 | 53.774 | 97.442 | 1.00 | 43.26 | C |
| ATOM | 14338 | O | ASP | G | 156 | −9.531 | 52.626 | 97.496 | 1.00 | 39.73 | O |
| ATOM | 14339 | N | ALA | G | 157 | −11.197 | 54.142 | 97.782 | 1.00 | 42.58 | N |
| ATOM | 14341 | CA | ALA | G | 157 | −12.231 | 53.250 | 98.261 | 1.00 | 40.81 | C |
| ATOM | 14343 | CB | ALA | G | 157 | −12.297 | 53.232 | 99.793 | 1.00 | 42.46 | C |
| ATOM | 14347 | C | ALA | G | 157 | −13.585 | 53.660 | 97.696 | 1.00 | 38.65 | C |
| ATOM | 14348 | O | ALA | G | 157 | −13.985 | 54.819 | 97.595 | 1.00 | 31.71 | O |
| ATOM | 14349 | N | VAL | G | 158 | −14.312 | 52.651 | 97.280 | 1.00 | 47.56 | N |
| ATOM | 14351 | CA | VAL | G | 158 | −15.556 | 52.952 | 96.629 | 1.00 | 51.33 | C |
| ATOM | 14353 | CB | VAL | G | 158 | −15.696 | 52.234 | 95.310 | 1.00 | 49.58 | C |
| ATOM | 14355 | CG1 | VAL | G | 158 | −15.640 | 50.729 | 95.557 | 1.80 | 44.81 | C |
| ATOM | 14359 | CG2 | VAL | G | 158 | −16.990 | 52.714 | 94.660 | 1.00 | 47.61 | C |
| ATOM | 14363 | C | VAL | G | 158 | −16.597 | 52.461 | 97.597 | 1.00 | 49.23 | C |
| ATOM | 14364 | O | VAL | G | 158 | −16.324 | 51.470 | 98.298 | 1.00 | 31.23 | O |
| ATOM | 14365 | N | VAL | G | 159 | −17.675 | 53.257 | 97.711 | 1.00 | 44.43 | N |
| ATOM | 14367 | CA | VAL | G | 159 | −18.807 | 52.909 | 98.539 | 1.00 | 40.38 | C |
| ATOM | 14369 | CB | VAL | G | 159 | −19.296 | 53.980 | 99.532 | 1.00 | 41.38 | C |
| ATOM | 14371 | CG1 | VAL | G | 159 | −20.309 | 53.360 | 100.455 | 1.00 | 54.96 | C |
| ATOM | 14375 | CG2 | VAL | G | 159 | −18.211 | 54.443 | 100.474 | 1.00 | 47.22 | C |
| ATOM | 14379 | C | VAL | G | 159 | −19.913 | 52.534 | 97.620 | 1.00 | 38.50 | C |
| ATOM | 14380 | O | VAL | G | 159 | −20.400 | 53.378 | 96.876 | 1.00 | 43.39 | O |
| ATOM | 14381 | N | ILE | G | 160 | −20.265 | 51.245 | 97.674 | 1.00 | 48.70 | N |
| ATOM | 14383 | CA | ILE | G | 160 | −21.250 | 50.648 | 96.775 | 1.00 | 52.89 | C |
| ATOM | 14385 | CB | ILE | G | 160 | −20.840 | 49.225 | 96.318 | 1.00 | 53.54 | C |
| ATOM | 14387 | CG1 | ILE | G | 160 | −19.490 | 49.261 | 95.620 | 1.00 | 52.89 | C |
| ATOM | 14390 | CD1 | ILE | G | 160 | −18.959 | 47.922 | 95.180 | 1.00 | 62.36 | C |
| ATOM | 14394 | CG2 | ILE | G | 160 | −21.842 | 48.692 | 95.307 | 1.00 | 53.02 | C |
| ATOM | 14398 | C | ILE | G | 160 | −22.555 | 50.584 | 97.541 | 1.00 | 51.62 | C |
| ATOM | 14399 | O | ILE | G | 160 | −22.594 | 50.014 | 98.641 | 1.00 | 42.40 | O |
| ATOM | 14400 | N | SER | G | 161 | −23.545 | 51.299 | 97.007 | 1.00 | 51.28 | N |
| ATOM | 14402 | CA | SER | G | 161 | −24.906 | 51.257 | 97.517 | 1.00 | 57.34 | C |
| ATOM | 14404 | CB | SER | G | 161 | −25.303 | 52.575 | 98.156 | 1.00 | 52.84 | C |
| ATOM | 14407 | OG | SER | G | 161 | −24.140 | 53.225 | 98.632 | 1.00 | 75.93 | O |
| ATOM | 14409 | C | SER | G | 161 | −25.911 | 50.873 | 96.442 | 1.00 | 63.66 | C |
| ATOM | 14410 | O | SER | G | 161 | −26.176 | 51.608 | 95.486 | 1.00 | 52.36 | O |
| ATOM | 14411 | N | CYS | G | 162 | −26.474 | 49.686 | 96.633 | 1.00 | 72.12 | N |
| ATOM | 14413 | CA | CYS | G | 162 | −27.537 | 49.217 | 95.764 | 1.00 | 77.24 | C |
| ATOM | 14415 | CB | CYS | G | 162 | −27.150 | 47.851 | 95.204 | 1.00 | 77.23 | C |
| ATOM | 14418 | SG | CYS | G | 162 | −28.434 | 46.630 | 95.422 | 1.00 | 109.64 | S |
| ATOM | 14419 | C | CYS | G | 162 | −28.884 | 49.211 | 96.497 | 1.00 | 70.66 | C |
| ATOM | 14420 | O | CYS | G | 162 | −29.023 | 48.612 | 97.557 | 1.00 | 71.31 | O |
| ATOM | 14421 | N | ALA | G | 163 | −29.841 | 49.953 | 95.946 | 1.00 | 68.84 | N |
| ATOM | 14423 | CA | ALA | G | 163 | −31.267 | 49.847 | 96.275 | 1.00 | 74.38 | C |
| ATOM | 14425 | CB | ALA | G | 163 | −31.864 | 51.248 | 96.375 | 1.00 | 72.65 | C |
| ATOM | 14429 | C | ALA | G | 163 | −32.059 | 49.031 | 95.232 | 1.00 | 75.01 | C |
| ATOM | 14430 | O | ALA | G | 163 | −31.476 | 48.458 | 94.308 | 1.00 | 79.54 | O |
| ATOM | 14431 | N | LYS | G | 164 | −33.385 | 49.011 | 95.337 | 1.00 | 71.72 | N |
| ATOM | 14433 | CA | LYS | G | 164 | −34.206 | 48.514 | 94.232 | 1.00 | 71.50 | C |
| ATOM | 14435 | CB | LYS | G | 164 | −35.673 | 48.444 | 94.657 | 1.00 | 78.10 | C |
| ATOM | 14438 | CG | LYS | G | 164 | −36.469 | 47.298 | 94.041 | 1.00 | 76.61 | C |
| ATOM | 14441 | CD | LYS | G | 164 | −37.781 | 47.069 | 94.794 | 1.00 | 72.13 | C |
| ATOM | 14444 | CE | LYS | G | 164 | −38.912 | 47.971 | 94.293 | 1.00 | 68.19 | C |
| ATOM | 14447 | NZ | LYS | G | 164 | −40.215 | 47.229 | 94.240 | 1.00 | 49.56 | N |
| ATOM | 14451 | C | LYS | G | 164 | −34.077 | 49.311 | 92.928 | 1.00 | 66.56 | C |
| ATOM | 14452 | O | LYS | G | 164 | −33.958 | 48.738 | 91.865 | 1.00 | 64.23 | O |
| ATOM | 14453 | N | ASP | G | 165 | −34.100 | 50.633 | 92.971 | 1.00 | 69.93 | N |
| ATOM | 14455 | CA | ASP | G | 165 | −34.087 | 51.412 | 91.730 | 1.00 | 73.04 | C |
| ATOM | 14457 | CB | ASP | G | 165 | −34.503 | 52.879 | 91.975 | 1.00 | 81.95 | C |
| ATOM | 14460 | CG | ASP | G | 165 | −33.487 | 53.695 | 92.821 | 1.00 | 93.64 | C |
| ATOM | 14461 | OD1 | ASP | G | 165 | −33.515 | 53.627 | 94.075 | 1.00 | 103.74 | O |
| ATOM | 14462 | OD2 | ASP | G | 165 | −32.692 | 54.524 | 92.324 | 1.00 | 101.26 | O |
| ATOM | 14463 | C | ASP | G | 165 | −32.772 | 51.388 | 90.950 | 1.00 | 70.19 | C |
| ATOM | 14464 | O | ASP | G | 165 | −32.745 | 51.774 | 89.780 | 1.00 | 65.36 | O |
| ATOM | 14465 | N | GLY | G | 166 | −31.666 | 51.033 | 91.600 | 1.00 | 64.15 | N |
| ATOM | 14467 | CA | GLY | G | 166 | −30.377 | 51.275 | 90.988 | 1.00 | 58.95 | C |
| ATOM | 14470 | C | GLY | G | 166 | −29.192 | 51.150 | 91.911 | 1.00 | 59.33 | C |
| ATOM | 14471 | O | GLY | G | 166 | −29.319 | 51.091 | 93.125 | 1.00 | 58.82 | O |
| ATOM | 14472 | N | VAL | G | 167 | −28.013 | 51.097 | 91.310 | 1.00 | 57.58 | N |
| ATOM | 14474 | CA | VAL | G | 167 | −26.791 | 50.808 | 92.047 | 1.00 | 55.81 | C |
| ATOM | 14476 | CB | VAL | G | 167 | −26.081 | 49.515 | 91.537 | 1.00 | 48.77 | C |
| ATOM | 14478 | CG1 | VAL | G | 167 | −25.701 | 49.641 | 90.110 | 1.00 | 54.52 | C |
| ATOM | 14482 | CG2 | VAL | G | 167 | −24.844 | 49.203 | 92.335 | 1.00 | 51.07 | C |
| ATOM | 14486 | C | VAL | G | 167 | −25.895 | 52.049 | 91.967 | 1.00 | 58.06 | C |
| ATOM | 14487 | O | VAL | G | 167 | −25.872 | 52.763 | 90.957 | 1.00 | 50.90 | O |
| ATOM | 14488 | N | LYS | G | 168 | −25.241 | 52.341 | 93.088 | 1.00 | 55.41 | N |
| ATOM | 14490 | CA | LYS | G | 168 | −24.387 | 53.492 | 93.174 | 1.00 | 52.82 | C |
| ATOM | 14492 | CB | LYS | G | 168 | −25.067 | 54.568 | 94.002 | 1.00 | 50.75 | C |
| ATOM | 14495 | CG | LYS | G | 168 | −24.675 | 55.987 | 93.602 | 1.00 | 57.32 | C |
| ATOM | 14498 | CD | LYS | G | 168 | −25.149 | 57.019 | 94.605 | 1.00 | 59.46 | C |
| ATOM | 14501 | CE | LYS | G | 168 | −24.677 | 56.693 | 96.013 | 1.00 | 77.03 | C |
| ATOM | 14504 | NZ | LYS | G | 168 | −25.120 | 57.745 | 96.989 | 1.00 | 97.25 | N |
| ATOM | 14508 | C | LYS | G | 168 | −22.970 | 53.213 | 93.702 | 1.00 | 59.47 | C |
| ATOM | 14509 | O | LYS | G | 168 | −22.732 | 52.374 | 94.589 | 1.00 | 54.97 | O |
| ATOM | 14510 | N | PHE | G | 169 | −22.063 | 54.035 | 93.176 | 1.00 | 57.75 | N |
| ATOM | 14512 | CA | PHE | G | 169 | −20.628 | 53.869 | 93.276 | 1.00 | 53.83 | C |
| ATOM | 14514 | CB | PHE | G | 169 | −20.080 | 53.454 | 91.923 | 1.00 | 51.14 | C |
| ATOM | 14517 | CG | PHE | G | 169 | −20.399 | 52.069 | 91.545 | 1.00 | 38.66 | C |
| ATOM | 14518 | CD1 | PHE | G | 169 | −21.409 | 51.820 | 90.640 | 1.00 | 45.85 | C |
| ATOM | 14520 | CE1 | PHE | G | 169 | −21.674 | 50.560 | 90.238 | 1.00 | 39.14 | C |
| ATOM | 14522 | CZ | PHE | G | 169 | −21.004 | 49.504 | 90.819 | 1.00 | 44.20 | C |
| ATOM | 14524 | CE2 | PHE | G | 169 | −19.965 | 49.735 | 91.692 | 1.00 | 56.49 | C |
| ATOM | 14526 | CD2 | PHE | G | 169 | −19.650 | 51.027 | 92.038 | 1.00 | 46.27 | C |
| ATOM | 14528 | C | PHE | G | 169 | −20.028 | 55.237 | 93.507 | 1.00 | 54.03 | C |
| ATOM | 14529 | O | PHE | G | 169 | −19.989 | 56.035 | 92.564 | 1.00 | 54.99 | O |
| ATOM | 14530 | N | SER | G | 170 | −19.464 | 55.456 | 94.692 | 1.00 | 53.80 | N |
| ATOM | 14532 | CA | SER | G | 170 | −19.034 | 56.789 | 95.141 | 1.00 | 50.63 | C |
| ATOM | 14534 | CB | SER | G | 170 | −19.977 | 57.244 | 96.239 | 1.00 | 52.37 | C |
| ATOM | 14537 | OG | SER | G | 170 | −20.308 | 56.113 | 97.028 | 1.00 | 62.10 | O |
| ATOM | 14539 | C | SER | G | 170 | −17.650 | 56.696 | 95.748 | 1.00 | 46.10 | C |
| ATOM | 14540 | O | SER | G | 170 | −17.337 | 55.781 | 96.513 | 1.00 | 43.75 | O |
| ATOM | 14541 | N | ALA | G | 171 | −16.790 | 57.642 | 95.424 | 1.00 | 47.46 | N |
| ATOM | 14543 | CA | ALA | G | 171 | −15.605 | 57.792 | 96.256 | 1.00 | 47.17 | C |
| ATOM | 14545 | CB | ALA | G | 171 | −14.439 | 57.018 | 95.714 | 1.00 | 48.77 | C |
| ATOM | 14549 | C | ALA | G | 171 | −15.279 | 59.259 | 96.447 | 1.00 | 53.18 | C |
| ATOM | 14550 | O | ALA | G | 171 | −15.965 | 60.139 | 95.909 | 1.00 | 57.32 | O |
| ATOM | 14551 | N | SER | G | 172 | −14.314 | 59.501 | 97.329 | 1.00 | 59.78 | N |
| ATOM | 14553 | CA | SER | G | 172 | −13.925 | 60.853 | 97.724 | 1.00 | 55.33 | C |
| ATOM | 14555 | CB | SER | G | 172 | −14.688 | 61.239 | 98.985 | 1.00 | 56.79 | C |
| ATOM | 14558 | OG | SER | G | 172 | −13.863 | 61.035 | 100.121 | 1.00 | 58.97 | O |
| ATOM | 14560 | C | SER | G | 172 | −12.429 | 60.859 | 98.016 | 1.00 | 54.54 | C |
| ATOM | 14561 | O | SER | G | 172 | −11.887 | 59.884 | 98.546 | 1.00 | 61.64 | O |
| ATOM | 14562 | N | GLY | G | 173 | −11.766 | 61.947 | 97.632 | 1.00 | 55.64 | N |
| ATOM | 14564 | CA | GLY | G | 173 | −10.344 | 62.162 | 97.865 | 1.00 | 49.97 | C |
| ATOM | 14567 | C | GLY | G | 173 | −9.976 | 63.649 | 97.875 | 1.00 | 60.56 | C |
| ATOM | 14568 | O | GLY | G | 173 | −10.831 | 64.582 | 98.074 | 1.00 | 58.61 | O |
| ATOM | 14569 | N | GLU | G | 174 | −8.683 | 63.888 | 97.635 | 1.00 | 63.16 | N |
| ATOM | 14571 | CA | GLU | G | 174 | −8.115 | 65.235 | 97.589 | 1.00 | 65.27 | C |
| ATOM | 14573 | CB | GLU | G | 174 | −6.610 | 65.176 | 97.325 | 1.00 | 72.09 | C |
| ATOM | 14576 | CG | GLU | G | 174 | −5.816 | 66.256 | 98.048 | 1.00 | 90.86 | C |
| ATOM | 14579 | CD | GLU | G | 174 | −5.465 | 65.871 | 99.482 | 1.00 | 115.28 | C |
| ATOM | 14580 | OE1 | GLU | G | 174 | −6.039 | 66.463 | 100.429 | 1.00 | 109.77 | O |
| ATOM | 14581 | OE2 | GLU | G | 174 | −4.611 | 64.971 | 99.667 | 1.00 | 127.55 | O |
| ATOM | 14582 | C | GLU | G | 174 | −8.800 | 66.152 | 96.573 | 1.00 | 65.39 | C |
| ATOM | 14583 | O | GLU | G | 174 | −9.335 | 67.194 | 96.974 | 1.00 | 59.23 | O |
| ATOM | 14584 | N | LEU | G | 175 | −8.850 | 65.737 | 95.299 | 1.00 | 64.49 | N |
| ATOM | 14586 | CA | LEU | G | 175 | −9.425 | 66.575 | 94.239 | 1.00 | 67.69 | C |
| ATOM | 14588 | CB | LEU | G | 175 | −9.083 | 66.083 | 92.833 | 1.00 | 69.96 | C |
| ATOM | 14591 | CG | LEU | G | 175 | −9.268 | 64.615 | 92.484 | 1.00 | 85.94 | C |
| ATOM | 14593 | CD1 | LEU | G | 175 | −8.907 | 64.447 | 91.011 | 1.00 | 81.66 | C |
| ATOM | 14597 | CD2 | LEU | G | 175 | −8.393 | 63.724 | 93.396 | 1.00 | 96.41 | C |
| ATOM | 14601 | C | LEU | G | 175 | −10.926 | 66.768 | 94.337 | 1.00 | 60.93 | C |
| ATOM | 14602 | O | LEU | G | 175 | −11.470 | 67.780 | 93.944 | 1.00 | 57.97 | O |
| ATOM | 14603 | N | GLY | G | 176 | −11.597 | 65.806 | 94.936 | 1.00 | 64.42 | N |
| ATOM | 14605 | CA | GLY | G | 176 | −12.875 | 66.093 | 95.555 | 1.00 | 64.02 | C |
| ATOM | 14608 | C | GLY | G | 176 | −13.671 | 64.809 | 95.642 | 1.00 | 57.23 | C |
| ATOM | 14609 | O | GLY | G | 176 | −13.156 | 63.771 | 96.058 | 1.00 | 55.04 | O |
| ATOM | 14610 | N | ASN | G | 177 | −14.919 | 64.842 | 95.206 | 1.00 | 46.76 | N |
| ATOM | 14612 | CA | ASN | G | 177 | −15.719 | 63.633 | 95.355 | 1.00 | 54.75 | C |
| ATOM | 14614 | CB | ASN | G | 177 | −16.503 | 63.654 | 96.681 | 1.00 | 54.53 | C |
| ATOM | 14617 | CG | ASN | G | 177 | −17.459 | 64.825 | 96.772 | 1.00 | 64.16 | C |
| ATOM | 14618 | OD1 | ASN | G | 177 | −18.529 | 64.830 | 96.154 | 1.00 | 40.56 | O |
| ATOM | 14619 | ND2 | ASN | G | 177 | −17.020 | 65.881 | 97.459 | 1.00 | 98.22 | N |
| ATOM | 14622 | C | ASN | G | 177 | −16.624 | 63.325 | 94.157 | 1.00 | 53.15 | C |
| ATOM | 14623 | O | ASN | G | 177 | −16.937 | 64.185 | 93.305 | 1.00 | 52.58 | O |
| ATOM | 14624 | N | GLY | G | 178 | −16.997 | 62.054 | 94.080 | 1.00 | 49.28 | N |
| ATOM | 14626 | CA | GLY | G | 178 | −17.777 | 61.585 | 92.950 | 1.00 | 53.77 | C |
| ATOM | 14629 | C | GLY | G | 178 | −18.774 | 60.486 | 93.261 | 1.00 | 49.32 | C |
| ATOM | 14630 | O | GLY | G | 178 | −18.516 | 59.559 | 94.075 | 1.00 | 39.80 | O |
| ATOM | 14631 | N | ASN | G | 179 | −19.896 | 60.563 | 92.552 | 1.00 | 46.42 | N |
| ATOM | 14633 | CA | ASN | G | 179 | −20.813 | 59.422 | 92.512 | 1.00 | 56.15 | C |
| ATOM | 14635 | CB | ASN | G | 179 | −21.957 | 59.557 | 93.537 | 1.00 | 55.35 | C |
| ATOM | 14638 | CG | ASN | G | 179 | −22.201 | 60.994 | 93.940 | 1.00 | 73.06 | C |
| ATOM | 14639 | OD1 | ASN | G | 179 | −23.028 | 61.691 | 93.338 | 1.00 | 92.57 | O |
| ATOM | 14640 | ND2 | ASN | G | 179 | −21.433 | 61.472 | 94.918 | 1.00 | 70.07 | N |
| ATOM | 14643 | C | ASN | G | 179 | −21.329 | 58.999 | 91.132 | 1.00 | 55.74 | C |
| ATOM | 14644 | O | ASN | G | 179 | −21.781 | 59.820 | 90.298 | 1.00 | 49.30 | O |
| ATOM | 14645 | N | ILE | G | 180 | −21.272 | 57.679 | 90.941 | 1.00 | 53.84 | N |
| ATOM | 14647 | CA | ILE | G | 180 | −21.790 | 57.018 | 89.741 | 1.00 | 57.79 | C |
| ATOM | 14649 | CB | ILE | G | 180 | −20.657 | 56.205 | 89.098 | 1.00 | 57.19 | C |
| ATOM | 14651 | CG1 | ILE | G | 180 | −19.393 | 57.065 | 88.963 | 1.00 | 68.57 | C |
| ATOM | 14654 | CD1 | ILE | G | 180 | −18.285 | 56.408 | 88.147 | 1.00 | 45.92 | C |
| ATOM | 14658 | CG2 | ILE | G | 180 | −21.040 | 55.730 | 87.724 | 1.00 | 46.99 | C |
| ATOM | 14662 | C | ILE | G | 180 | −23.027 | 56.126 | 90.020 | 1.00 | 60.35 | C |
| ATOM | 14663 | O | ILE | G | 180 | −22.929 | 55.086 | 90.679 | 1.00 | 53.23 | O |
| ATOM | 14664 | N | LYS | G | 181 | −24.177 | 56.563 | 89.492 | 1.00 | 59.01 | N |
| ATOM | 14666 | CA | LYS | G | 181 | −25.475 | 55.909 | 89.645 | 1.00 | 61.08 | C |
| ATOM | 14668 | CB | LYS | G | 181 | −26.533 | 56.936 | 90.072 | 1.00 | 58.51 | C |
| ATOM | 14671 | CG | LYS | G | 181 | −27.725 | 56.320 | 90.809 | 1.00 | 83.10 | C |
| ATOM | 14674 | CD | LYS | G | 181 | −29.083 | 56.632 | 90.133 | 1.00 | 97.91 | C |
| ATOM | 14677 | CE | LYS | G | 181 | −30.297 | 56.083 | 90.902 | 1.00 | 86.17 | C |
| ATOM | 14680 | NZ | LYS | G | 181 | −31.603 | 56.719 | 90.523 | 1.00 | 79.08 | N |
| ATOM | 14684 | C | LYS | G | 181 | −25.912 | 55.189 | 88.350 | 1.00 | 64.68 | C |
| ATOM | 14685 | O | LYS | G | 181 | −25.959 | 55.758 | 87.243 | 1.00 | 62.96 | O |
| ATOM | 14686 | N | LEU | G | 192 | −26.185 | 53.897 | 88.504 | 1.00 | 63.26 | N |
| ATOM | 14688 | CA | LEU | G | 182 | −26.715 | 53.072 | 87.422 | 1.00 | 61.01 | C |
| ATOM | 14690 | CB | LEU | G | 182 | −25.839 | 51.833 | 87.258 | 1.00 | 55.44 | C |
| ATOM | 14693 | CG | LEU | G | 182 | −24.531 | 51.970 | 86.484 | 1.00 | 59.79 | C |
| ATOM | 14695 | CD1 | LEU | G | 182 | −23.863 | 50.587 | 86.386 | 1.00 | 73.60 | C |
| ATOM | 14699 | CD2 | LEU | G | 182 | −24.790 | 52.540 | 85.113 | 1.00 | 64.31 | C |
| ATOM | 14703 | C | LEU | G | 182 | −28.132 | 52.627 | 87.788 | 1.00 | 60.11 | C |
| ATOM | 14704 | O | LEU | G | 182 | −28.324 | 51.966 | 88.813 | 1.00 | 50.97 | O |
| ATOM | 14705 | N | SER | G | 183 | −29.118 | 53.010 | 86.981 | 1.00 | 59.59 | N |
| ATOM | 14707 | CA | SER | G | 183 | −30.506 | 52.663 | 87.278 | 1.00 | 67.35 | C |
| ATOM | 14709 | CB | SER | G | 183 | −31.440 | 53.845 | 86.982 | 1.00 | 70.77 | C |
| ATOM | 14712 | OG | SER | G | 183 | −31.443 | 54.236 | 85.621 | 1.00 | 58.99 | O |
| ATOM | 14714 | C | SER | G | 183 | −30.954 | 51.334 | 86.629 | 1.00 | 72.87 | C |
| ATOM | 14715 | O | SER | G | 183 | −30.337 | 50.904 | 85.659 | 1.00 | 77.20 | O |
| ATOM | 14716 | N | GLN | G | 184 | −31.886 | 50.595 | 87.247 | 1.00 | 76.81 | N |
| ATOM | 14718 | CA | GLN | G | 184 | −32.178 | 49.225 | 86.814 | 1.00 | 78.57 | C |
| ATOM | 14720 | CB | GLN | G | 184 | −33.051 | 48.457 | 87.832 | 1.00 | 79.91 | C |
| ATOM | 14723 | CG | GLN | G | 184 | −33.384 | 46.960 | 87.458 | 1.00 | 79.48 | C |
| ATOM | 14726 | CD | GLN | G | 184 | −33.937 | 46.021 | 88.586 | 1.00 | 68.14 | C |
| ATOM | 14727 | OE1 | GLN | G | 184 | −34.246 | 46.432 | 89.701 | 1.00 | 81.04 | O |
| ATOM | 14728 | NE2 | GLN | G | 184 | −34.085 | 44.750 | 88.254 | 1.00 | 69.96 | N |
| ATOM | 14731 | C | GLN | G | 184 | −32.892 | 49.420 | 85.484 | 1.00 | 83.79 | C |
| ATOM | 14732 | O | GLN | G | 184 | −33.721 | 50.321 | 85.364 | 1.00 | 78.11 | O |
| ATOM | 14733 | N | THR | G | 185 | −32.493 | 48.675 | 84.457 | 1.00 | 91.73 | N |
| ATOM | 14735 | CA | THR | G | 185 | −33.100 | 48.855 | 83.137 | 1.00 | 105.00 | C |
| ATOM | 14737 | CB | THR | G | 185 | −32.590 | 50.147 | 82.446 | 1.00 | 109.21 | C |
| ATOM | 14739 | OG1 | THR | G | 185 | −33.410 | 50.475 | 81.315 | 1.00 | 110.77 | O |
| ATOM | 14741 | CG2 | THR | G | 185 | −31.222 | 49.930 | 81.812 | 1.00 | 115.31 | C |
| ATOM | 14745 | C | THR | G | 185 | −32.810 | 47.676 | 82.229 | 1.00 | 111.17 | C |
| ATOM | 14746 | O | THR | G | 185 | −31.889 | 46.906 | 82.491 | 1.00 | 111.47 | O |
| ATOM | 14747 | N | SER | G | 186 | −33.520 | 47.628 | 81.104 | 1.00 | 121.03 | N |
| ATOM | 14749 | CA | SER | G | 186 | −34.370 | 46.493 | 80.728 | 1.00 | 129.61 | C |
| ATOM | 14751 | CB | SER | G | 186 | −34.864 | 45.730 | 81.967 | 1.00 | 131.16 | C |
| ATOM | 14754 | OG | SER | G | 186 | −36.015 | 46.336 | 82.535 | 1.00 | 134.00 | O |
| ATOM | 14756 | C | SER | G | 186 | −35.567 | 47.019 | 79.930 | 1.00 | 133.53 | C |
| ATOM | 14757 | O | SER | G | 186 | −36.432 | 46.247 | 79.483 | 1.00 | 136.34 | O |
| ATOM | 14758 | N | ASN | G | 187 | −35.601 | 48.349 | 79.797 | 1.00 | 133.76 | N |
| ATOM | 14760 | CA | ASN | G | 187 | −36.474 | 49.056 | 78.861 | 1.00 | 127.74 | C |
| ATOM | 14762 | CB | ASN | G | 187 | −37.373 | 50.021 | 79.645 | 1.00 | 124.02 | C |
| ATOM | 14765 | CG | ASN | G | 187 | −37.387 | 49.732 | 81.147 | 1.00 | 96.29 | C |
| ATOM | 14766 | OD1 | ASN | G | 187 | −36.866 | 50.495 | 81.928 | 1.00 | 62.80 | O |
| ATOM | 14767 | ND2 | ASN | G | 187 | −37.976 | 48.629 | 81.545 | 1.00 | 90.88 | N |
| ATOM | 14770 | C | ASN | G | 187 | −35.674 | 49.748 | 77.736 | 1.00 | 130.64 | C |
| ATOM | 14771 | O | ASN | G | 187 | −35.491 | 50.968 | 77.685 | 1.00 | 120.77 | O |
| ATOM | 14772 | N | VAL | G | 188 | −35.232 | 48.911 | 76.804 | 1.00 | 138.94 | N |
| ATOM | 14774 | CA | VAL | G | 188 | −34.018 | 49.127 | 76.019 | 1.00 | 142.65 | C |
| ATOM | 14776 | CB | VAL | G | 188 | −32.743 | 48.864 | 76.882 | 1.00 | 138.21 | C |
| ATOM | 14778 | CG1 | VAL | G | 188 | −32.583 | 49.971 | 77.895 | 1.00 | 132.17 | C |
| ATOM | 14782 | CG2 | VAL | G | 188 | −32.804 | 47.507 | 77.602 | 1.00 | 126.31 | C |
| ATOM | 14786 | C | VAL | G | 188 | −34.114 | 48.226 | 74.760 | 1.00 | 151.19 | C |
| ATOM | 14787 | O | VAL | G | 188 | −35.216 | 48.055 | 74.220 | 1.00 | 148.25 | O |
| ATOM | 14788 | N | ASP | G | 189 | −32.995 | 47.657 | 74.291 | 1.00 | 159.58 | N |
| ATOM | 14790 | CA | ASP | G | 189 | −32.994 | 46.606 | 73.246 | 1.00 | 162.11 | C |
| ATOM | 14792 | CB | ASP | G | 189 | −32.170 | 47.037 | 72.002 | 1.00 | 161.39 | C |
| ATOM | 14795 | CG | ASP | G | 189 | −30.680 | 47.274 | 72.303 | 1.00 | 158.07 | C |
| ATOM | 14796 | OD1 | ASP | G | 189 | −30.238 | 48.444 | 72.312 | 1.00 | 140.42 | O |
| ATOM | 14797 | OD2 | ASP | G | 189 | −29.851 | 46.357 | 72.511 | 1.00 | 153.08 | O |
| ATOM | 14798 | C | ASP | G | 189 | −32.571 | 45.191 | 73.731 | 1.00 | 162.86 | C |
| ATOM | 14799 | O | ASP | G | 189 | −31.374 | 44.918 | 73.903 | 1.00 | 164.80 | O |
| ATOM | 14800 | N | LYS | G | 190 | −33.545 | 44.294 | 73.928 | 1.00 | 158.68 | N |
| ATOM | 14802 | CA | LYS | G | 190 | −33.287 | 42.927 | 74.405 | 1.00 | 154.93 | C |
| ATOM | 14804 | CB | LYS | G | 190 | −32.771 | 42.051 | 73.248 | 1.00 | 153.30 | C |
| ATOM | 14807 | CG | LYS | G | 190 | −32.516 | 42.853 | 71.956 | 1.00 | 156.85 | C |
| ATOM | 14810 | CD | LYS | G | 190 | −31.833 | 42.083 | 70.018 | 1.00 | 157.05 | C |
| ATOM | 14813 | CE | LYS | G | 190 | −31.384 | 43.047 | 69.704 | 1.00 | 154.06 | C |
| ATOM | 14816 | NZ | LYS | G | 190 | −30.549 | 42.389 | 68.655 | 1.00 | 149.46 | N |
| ATOM | 14820 | C | LYS | G | 190 | −32.349 | 42.927 | 75.634 | 1.00 | 153.29 | C |
| ATOM | 14821 | O | LYS | G | 190 | −32.601 | 43.647 | 76.608 | 1.00 | 151.04 | O |
| ATOM | 14822 | N | GLU | G | 191 | −31.272 | 42.141 | 75.601 | 1.00 | 152.07 | N |
| ATOM | 14824 | CA | GLU | G | 191 | −30.255 | 42.154 | 76.666 | 1.00 | 149.83 | C |
| ATOM | 14826 | CB | GLU | G | 191 | −29.691 | 40.742 | 76.915 | 1.00 | 150.37 | C |
| ATOM | 14829 | CG | GLU | G | 191 | −29.382 | 40.403 | 78.373 | 1.00 | 148.04 | C |
| ATOM | 14832 | CD | GLU | G | 191 | −30.327 | 39.356 | 78.941 | 1.00 | 145.00 | C |
| ATOM | 14833 | OE1 | GLU | G | 191 | −30.469 | 38.283 | 78.317 | 1.00 | 148.54 | O |
| ATOM | 14834 | OE2 | GLU | G | 191 | −30.953 | 39.603 | 79.995 | 1.00 | 133.24 | O |
| ATOM | 14835 | C | GLU | G | 191 | −29.103 | 43.130 | 76.387 | 1.00 | 144.84 | C |
| ATOM | 14836 | O | GLU | G | 191 | −29.125 | 44.274 | 76.854 | 1.00 | 144.70 | O |
| ATOM | 14837 | N | GLU | G | 192 | −28.097 | 42.664 | 75.647 | 1.00 | 137.41 | N |
| ATOM | 14839 | CA | GLU | G | 192 | −26.818 | 43.368 | 75.527 | 1.00 | 131.03 | C |
| ATOM | 14841 | CB | GLU | G | 192 | −27.036 | 44.724 | 74.817 | 1.00 | 131.58 | C |
| ATOM | 14844 | CG | GLU | G | 192 | −26.527 | 44.821 | 73.381 | 1.00 | 131.53 | C |
| ATOM | 14847 | CD | GLU | G | 192 | −27.564 | 48.363 | 72.398 | 1.00 | 130.36 | C |
| ATOM | 14848 | OE1 | GLU | G | 192 | −27.757 | 44.739 | 71.326 | 1.00 | 123.48 | O |
| ATOM | 14849 | OE2 | GLU | G | 192 | −28.178 | 46.422 | 72.674 | 1.00 | 114.40 | O |
| ATOM | 14850 | C | GLU | G | 192 | −26.225 | 43.615 | 76.917 | 1.00 | 119.77 | C |
| ATOM | 14851 | O | GLU | G | 192 | −25.534 | 42.787 | 77.523 | 1.00 | 112.35 | O |
| ATOM | 14852 | N | GLU | G | 193 | −26.565 | 44.790 | 77.417 | 1.00 | 106.95 | N |
| ATOM | 14854 | CA | GLU | G | 193 | −25.709 | 45.508 | 78.323 | 1.00 | 101.59 | C |
| ATOM | 14856 | CB | GLU | G | 193 | −24.834 | 46.500 | 77.545 | 1.00 | 104.25 | C |
| ATOM | 14859 | CG | GLU | G | 193 | −24.856 | 47.935 | 78.076 | 1.00 | 114.41 | C |
| ATOM | 14862 | CD | GLU | G | 193 | −25.324 | 48.983 | 77.069 | 1.00 | 113.25 | C |
| ATOM | 14863 | OE1 | GLU | G | 193 | −25.471 | 50.161 | 77.475 | 1.00 | 96.78 | O |
| ATOM | 14864 | OE2 | GLU | G | 193 | −25.523 | 48.654 | 75.875 | 1.00 | 108.88 | O |
| ATOM | 14865 | C | GLU | G | 193 | −26.698 | 46.219 | 79.241 | 1.00 | 93.75 | C |
| ATOM | 14866 | O | GLU | G | 193 | −27.006 | 47.391 | 79.028 | 1.00 | 82.09 | O |
| ATOM | 14867 | N | ALA | G | 194 | −27.194 | 45.493 | 80.245 | 1.00 | 84.24 | N |
| ATOM | 14869 | CA | ALA | G | 194 | −28.181 | 46.011 | 81.172 | 1.00 | 74.51 | C |
| ATOM | 14871 | CB | ALA | G | 194 | −29.522 | 45.448 | 80.839 | 1.00 | 75.39 | C |
| ATOM | 14875 | C | ALA | G | 194 | −27.857 | 45.649 | 82.599 | 1.00 | 75.01 | C |
| ATOM | 14876 | O | ALA | G | 194 | −27.135 | 44.677 | 82.880 | 1.00 | 79.19 | O |
| ATOM | 14877 | N | VAL | G | 195 | −28.499 | 46.400 | 83.485 | 1.00 | 66.74 | N |
| ATOM | 14879 | CA | VAL | G | 195 | −28.348 | 46.226 | 84.919 | 1.00 | 63.50 | C |
| ATOM | 14881 | CB | VAL | G | 195 | −28.049 | 47.587 | 85.625 | 1.00 | 66.98 | C |
| ATOM | 14883 | CG1 | VAL | G | 195 | −27.532 | 47.373 | 87.072 | 1.00 | 59.29 | C |
| ATOM | 14887 | CG2 | VAL | G | 195 | −27.068 | 48.397 | 84.819 | 1.00 | 63.22 | C |
| ATOM | 14891 | C | VAL | G | 195 | −29.637 | 45.677 | 85.527 | 1.00 | 61.07 | C |
| ATOM | 14892 | O | VAL | G | 195 | −30.727 | 46.183 | 85.252 | 1.00 | 64.73 | O |
| ATOM | 14893 | N | THR | G | 196 | −29.492 | 44.713 | 86.429 | 1.00 | 54.86 | N |
| ATOM | 14895 | CA | THR | G | 196 | −30.634 | 44.119 | 87.100 | 1.00 | 58.01 | C |
| ATOM | 14897 | CB | THR | G | 196 | −31.168 | 42.861 | 86.325 | 1.00 | 60.99 | C |
| ATOM | 14899 | OG1 | THR | G | 196 | −30.103 | 41.979 | 85.974 | 1.00 | 50.33 | O |
| ATOM | 14901 | CG2 | THR | G | 196 | −31.659 | 43.263 | 84.933 | 1.00 | 54.78 | C |
| ATOM | 14905 | C | THR | G | 196 | −30.253 | 43.794 | 88.530 | 1.00 | 54.68 | C |
| ATOM | 14906 | O | THR | G | 196 | −29.249 | 43.141 | 88.765 | 1.00 | 58.21 | O |
| ATOM | 14907 | N | ILE | G | 197 | −31.026 | 44.307 | 89.480 | 1.00 | 52.15 | N |
| ATOM | 14909 | CA | ILE | G | 197 | −30.755 | 44.068 | 90.886 | 1.00 | 52.98 | C |
| ATOM | 14911 | CB | ILE | G | 197 | −30.913 | 45.387 | 91.643 | 1.00 | 56.92 | C |
| ATOM | 14913 | CG1 | ILE | G | 197 | −29.922 | 46.433 | 91.124 | 1.00 | 59.18 | C |
| ATOM | 14916 | CD1 | ILE | G | 197 | −30.539 | 47.538 | 90.321 | 1.00 | 55.00 | C |
| ATOM | 14920 | CG2 | ILE | G | 197 | −30.759 | 45.134 | 93.155 | 1.00 | 51.41 | C |
| ATOM | 14924 | C | ILE | G | 197 | −31.805 | 43.115 | 91.432 | 1.00 | 51.33 | C |
| ATOM | 14925 | O | ILE | G | 197 | −32.983 | 43.349 | 91.177 | 1.00 | 58.38 | O |
| ATOM | 14926 | N | GLU | G | 198 | −31.390 | 42.068 | 92.146 | 1.00 | 48.58 | N |
| ATOM | 14928 | CA | GLU | G | 198 | −32.238 | 41.334 | 93.087 | 1.00 | 59.15 | C |
| ATOM | 14930 | CB | GLU | G | 198 | −31.882 | 39.845 | 93.188 | 1.00 | 63.66 | C |
| ATOM | 14933 | CG | GLU | G | 198 | −31.863 | 39.091 | 91.863 | 1.00 | 86.92 | C |
| ATOM | 14936 | CD | GLU | G | 198 | −33.199 | 38.454 | 91.546 | 1.00 | 96.01 | C |
| ATOM | 14937 | OE1 | GLU | G | 198 | −33.545 | 37.520 | 92.296 | 1.00 | 97.86 | O |
| ATOM | 14938 | OE2 | GLU | G | 198 | −33.889 | 38.900 | 90.589 | 1.00 | 83.44 | O |
| ATOM | 14939 | C | GLU | G | 198 | −32.071 | 41.884 | 94.481 | 1.00 | 62.58 | C |
| ATOM | 14940 | O | GLU | G | 198 | −31.246 | 41.374 | 95.242 | 1.00 | 56.62 | O |
| ATOM | 14941 | N | MET | G | 199 | −32.907 | 42.873 | 94.803 | 1.00 | 71.25 | N |
| ATOM | 14943 | CA | MET | G | 199 | −32.963 | 43.528 | 96.117 | 1.00 | 72.70 | C |
| ATOM | 14945 | CB | MET | G | 199 | −33.760 | 44.830 | 95.977 | 1.00 | 70.87 | C |
| ATOM | 14948 | CG | MET | G | 199 | −33.148 | 46.000 | 96.714 | 1.00 | 77.42 | C |
| ATOM | 14951 | SD | MET | G | 199 | −33.634 | 46.139 | 98.441 | 1.00 | 86.41 | S |
| ATOM | 14952 | CE | MET | G | 199 | −34.327 | 47.853 | 98.310 | 1.00 | 92.54 | C |
| ATOM | 14956 | C | MET | G | 199 | −33.637 | 42.660 | 97.179 | 1.00 | 70.48 | C |
| ATOM | 14957 | O | MET | G | 199 | −34.717 | 42.148 | 96.948 | 1.00 | 75.42 | O |
| ATOM | 14958 | N | ASN | G | 200 | −33.035 | 42.503 | 98.348 | 1.00 | 72.46 | N |
| ATOM | 14960 | CA | ASN | G | 200 | −33.726 | 41.875 | 99.477 | 1.00 | 67.90 | C |
| ATOM | 14962 | CB | ASN | G | 200 | −33.024 | 40.606 | 99.981 | 1.00 | 68.90 | C |
| ATOM | 14965 | CG | ASN | G | 200 | −33.382 | 39.372 | 99.177 | 1.00 | 74.77 | C |
| ATOM | 14966 | OD1 | ASN | G | 200 | −34.074 | 39.471 | 98.164 | 1.00 | 62.32 | O |
| ATOM | 14967 | ND2 | ASN | G | 200 | −32.924 | 38.198 | 99.634 | 1.00 | 84.60 | N |
| ATOM | 14970 | C | ASN | G | 200 | −33.745 | 42.860 | 100.613 | 1.00 | 61.40 | C |
| ATOM | 14971 | O | ASN | G | 200 | −34.726 | 42.998 | 101.298 | 1.00 | 74.54 | O |
| ATOM | 14972 | N | GLU | G | 201 | −32.637 | 43.525 | 100.856 | 1.00 | 59.97 | N |
| ATOM | 14974 | CA | GLU | G | 201 | −32.686 | 44.773 | 101.588 | 1.00 | 58.67 | C |
| ATOM | 14976 | CB | GLU | G | 201 | −32.775 | 44.491 | 103.088 | 1.00 | 49.45 | C |
| ATOM | 14979 | CG | GLU | G | 201 | −31.762 | 43.470 | 103.561 | 1.00 | 51.52 | C |
| ATOM | 14982 | CD | GLU | G | 201 | −32.321 | 42.066 | 103.606 | 1.00 | 83.04 | C |
| ATOM | 14983 | OE1 | GLU | G | 201 | −31.848 | 41.142 | 102.895 | 1.00 | 77.75 | O |
| ATOM | 14984 | OE2 | GLU | G | 201 | −33.274 | 41.900 | 104.387 | 1.00 | 120.53 | O |
| ATOM | 14985 | C | GLU | G | 201 | −31.425 | 45.528 | 101.184 | 1.00 | 64.12 | C |
| ATOM | 14986 | O | GLU | G | 201 | −30.368 | 44.908 | 100.934 | 1.00 | 60.41 | O |
| ATOM | 14987 | N | PRO | G | 202 | −31.565 | 46.845 | 101.033 | 1.00 | 65.17 | N |
| ATOM | 14988 | CA | PRO | G | 202 | −30.460 | 47.695 | 100.575 | 1.00 | 66.59 | C |
| ATOM | 14990 | CB | PRO | G | 202 | −30.997 | 49.112 | 100.758 | 1.00 | 64.26 | C |
| ATOM | 14993 | CG | PRO | G | 202 | −32.450 | 48.946 | 100.574 | 1.00 | 66.60 | C |
| ATOM | 14996 | CD | PRO | G | 202 | −32.772 | 47.637 | 101.298 | 1.00 | 63.18 | C |
| ATOM | 14999 | C | PRO | G | 202 | −29.203 | 47.494 | 101.389 | 1.00 | 62.30 | C |
| ATOM | 15000 | O | PRO | G | 202 | −29.230 | 47.266 | 102.608 | 1.00 | 69.81 | O |
| ATOM | 15001 | N | VAL | G | 203 | −28.106 | 47.479 | 100.652 | 1.00 | 59.57 | N |
| ATOM | 15003 | CA | VAL | G | 203 | −26.783 | 47.423 | 101.235 | 1.00 | 63.60 | C |
| ATOM | 15005 | CB | VAL | G | 203 | −26.027 | 46.189 | 100.735 | 1.00 | 62.99 | C |
| ATOM | 15007 | CG1 | VAL | G | 203 | −24.520 | 46.454 | 100.830 | 1.00 | 87.59 | C |
| ATOM | 15011 | CG2 | VAL | G | 203 | −26.385 | 44.959 | 101.548 | 1.00 | 61.25 | C |
| ATOM | 15015 | C | VAL | G | 203 | −26.005 | 48.660 | 100.798 | 1.00 | 63.33 | C |
| ATOM | 15016 | O | VAL | G | 203 | −26.063 | 49.022 | 99.625 | 1.00 | 61.78 | O |
| ATOM | 15017 | N | GLN | G | 204 | −25.289 | 49.293 | 101.731 | 1.00 | 67.51 | N |
| ATOM | 15019 | CA | GLN | G | 204 | −24.178 | 50.207 | 101.425 | 1.00 | 63.79 | C |
| ATOM | 15021 | CB | GLN | G | 204 | −24.390 | 51.538 | 102.135 | 1.00 | 56.31 | C |
| ATOM | 15024 | CG | GLN | G | 204 | −23.516 | 52.640 | 101.596 | 1.00 | 72.38 | C |
| ATOM | 15027 | CD | GLN | G | 204 | −23.670 | 53.980 | 102.322 | 1.00 | 72.81 | C |
| ATOM | 15028 | OE1 | GLN | G | 204 | −24.235 | 54.921 | 101.765 | 1.00 | 72.31 | O |
| ATOM | 15029 | NE2 | GLN | G | 204 | −23.084 | 54.094 | 103.510 | 1.00 | 62.38 | N |
| ATOM | 15032 | C | GLN | G | 204 | −22.900 | 49.594 | 101.948 | 1.00 | 60.27 | C |
| ATOM | 15033 | O | GLN | G | 204 | −22.829 | 49.268 | 103.116 | 1.00 | 65.59 | O |
| ATOM | 15034 | N | LEU | G | 205 | −21.879 | 49.413 | 101.130 | 1.00 | 61.76 | N |
| ATOM | 15036 | CA | LEU | G | 205 | −20.616 | 48.939 | 101.707 | 1.00 | 57.96 | C |
| ATOM | 15038 | CB | LEU | G | 205 | −20.441 | 47.438 | 101.469 | 1.00 | 67.07 | C |
| ATOM | 15041 | CG | LEU | G | 205 | −21.236 | 46.418 | 102.312 | 1.00 | 82.89 | C |
| ATOM | 15043 | CD1 | LEU | G | 205 | −21.013 | 44.963 | 101.827 | 1.00 | 69.45 | C |
| ATOM | 15047 | CD2 | LEU | G | 205 | −20.873 | 46.541 | 103.806 | 1.00 | 99.32 | C |
| ATOM | 15051 | C | LEU | G | 205 | −19.428 | 49.688 | 101.124 | 1.00 | 52.03 | C |
| ATOM | 15052 | O | LEU | G | 205 | −19.507 | 50.314 | 100.064 | 1.00 | 49.75 | O |
| ATOM | 15053 | N | THR | G | 206 | −18.311 | 49.630 | 101.826 | 1.00 | 49.41 | N |
| ATOM | 15055 | CA | THR | G | 206 | −17.130 | 50.362 | 101.395 | 1.00 | 46.44 | C |
| ATOM | 15057 | CB | THR | G | 206 | −16.673 | 51.258 | 102.560 | 1.00 | 52.79 | C |
| ATOM | 15059 | OG1 | THR | G | 206 | −17.293 | 52.553 | 102.497 | 1.00 | 60.90 | O |
| ATOM | 15061 | CG2 | THR | G | 206 | −15.192 | 51.518 | 102.477 | 1.00 | 50.13 | C |
| ATOM | 15065 | C | THR | G | 206 | −16.037 | 49.331 | 101.080 | 1.00 | 46.26 | C |
| ATOM | 15066 | O | THR | G | 206 | −15.721 | 48.477 | 101.903 | 1.00 | 38.59 | O |
| ATOM | 15067 | N | PHE | G | 207 | −15.469 | 49.390 | 99.879 | 1.00 | 48.35 | N |
| ATOM | 15069 | CA | PHE | G | 207 | −14.376 | 48.499 | 99.508 | 1.00 | 45.07 | C |
| ATOM | 15071 | CB | PHE | G | 207 | −14.870 | 47.528 | 98.442 | 1.00 | 51.44 | C |
| ATOM | 15074 | CG | PHE | G | 207 | −16.005 | 46.661 | 98.900 | 1.00 | 52.43 | C |
| ATOM | 15075 | CD1 | PHE | G | 207 | −15.779 | 45.612 | 99.767 | 1.00 | 46.78 | C |
| ATOM | 15077 | CE1 | PHE | G | 207 | −16.818 | 44.874 | 100.207 | 1.00 | 51.15 | C |
| ATOM | 15079 | CZ | PHE | G | 207 | −18.100 | 45.135 | 99.759 | 1.00 | 46.30 | C |
| ATOM | 15081 | CE2 | PHE | G | 207 | −18.348 | 46.169 | 98.931 | 1.00 | 44.28 | C |
| ATOM | 15083 | CD2 | PHE | G | 207 | −17.303 | 46.943 | 98.503 | 1.00 | 61.92 | C |
| ATOM | 15085 | C | PHE | G | 207 | −13.161 | 49.233 | 98.962 | 1.00 | 43.83 | C |
| ATOM | 15086 | O | PHE | G | 207 | −13.253 | 50.366 | 98.490 | 1.00 | 43.90 | O |
| ATOM | 15087 | N | ALA | G | 208 | −12.024 | 48.556 | 98.953 | 1.00 | 41.18 | N |
| ATOM | 15089 | CA | ALA | G | 208 | −10.819 | 49.142 | 98.366 | 1.00 | 49.61 | C |
| ATOM | 15091 | CB | ALA | G | 208 | −9.584 | 48.479 | 98.975 | 1.00 | 48.08 | C |
| ATOM | 15095 | C | ALA | G | 208 | −10.746 | 49.034 | 96.833 | 1.00 | 53.10 | C |
| ATOM | 15096 | O | ALA | G | 208 | −10.795 | 47.924 | 96.291 | 1.00 | 60.38 | O |
| ATOM | 15097 | N | LEU | G | 209 | −10.566 | 50.156 | 96.136 | 1.00 | 52.42 | N |
| ATOM | 15099 | CA | LEU | G | 209 | −10.596 | 50.160 | 94.670 | 1.00 | 48.45 | C |
| ATOM | 15101 | CB | LEU | G | 209 | −10.562 | 51.586 | 94.127 | 1.00 | 42.22 | C |
| ATOM | 15104 | CG | LEU | G | 209 | −11.954 | 52.226 | 94.076 | 1.00 | 54.56 | C |
| ATOM | 15106 | CD1 | LEU | G | 209 | −11.885 | 53.675 | 93.654 | 1.00 | 67.50 | C |
| ATOM | 15110 | CD2 | LEU | G | 209 | −12.848 | 51.496 | 93.123 | 1.00 | 52.13 | C |
| ATOM | 15114 | C | LEU | G | 209 | −9.477 | 49.306 | 94.058 | 1.00 | 51.86 | C |
| ATOM | 15115 | O | LEU | G | 209 | −9.640 | 48.656 | 93.006 | 1.00 | 45.00 | O |
| ATOM | 15116 | N | ARG | G | 210 | −8.348 | 49.254 | 94.753 | 1.00 | 55.70 | N |
| ATOM | 15118 | CA | ARG | G | 210 | −7.145 | 48.704 | 94.136 | 1.00 | 60.05 | C |
| ATOM | 15120 | CB | ARG | G | 210 | −5.899 | 49.047 | 94.967 | 1.00 | 65.10 | C |
| ATOM | 15123 | CG | ARG | G | 210 | −4.586 | 48.326 | 94.634 | 1.00 | 64.76 | C |
| ATOM | 15126 | CD | ARG | G | 210 | −3.859 | 47.901 | 95.926 | 1.00 | 77.46 | C |
| ATOM | 15129 | NE | ARG | G | 210 | −2.677 | 47.060 | 95.738 | 1.00 | 83.39 | N |
| ATOM | 15131 | CZ | ARG | G | 210 | −1.675 | 47.331 | 94.900 | 1.00 | 79.07 | C |
| ATOM | 15132 | NH1 | ARG | G | 210 | −1.721 | 48.426 | 94.133 | 1.00 | 75.70 | N |
| ATOM | 15135 | NH2 | ARG | G | 210 | −0.640 | 46.485 | 94.829 | 1.00 | 47.40 | N |
| ATOM | 15138 | C | ARG | G | 210 | −7.335 | 47.204 | 93.969 | 1.00 | 56.83 | C |
| ATOM | 15139 | O | ARG | G | 210 | −6.953 | 46.611 | 92.949 | 1.00 | 57.69 | O |
| ATOM | 15140 | N | TYR | G | 211 | −8.008 | 46.605 | 94.945 | 1.00 | 52.02 | N |
| ATOM | 15142 | CA | TYR | G | 211 | −8.339 | 45.191 | 94.848 | 1.00 | 43.63 | C |
| ATOM | 15144 | CB | TYR | G | 211 | −8.812 | 44.683 | 96.210 | 1.00 | 40.90 | C |
| ATOM | 15147 | CG | TYR | G | 211 | −7.688 | 44.519 | 97.221 | 1.00 | 51.91 | C |
| ATOM | 15148 | CD1 | TYR | G | 211 | −6.921 | 43.359 | 97.260 | 1.00 | 51.61 | C |
| ATOM | 15150 | CE1 | TYR | G | 211 | −5.945 | 43.162 | 98.235 | 1.00 | 58.26 | C |
| ATOM | 15152 | CZ | TYR | G | 211 | −5.680 | 44.163 | 99.157 | 1.00 | 68.05 | C |
| ATOM | 15153 | OH | TYR | G | 211 | −4.682 | 43.993 | 100.111 | 1.00 | 51.21 | O |
| ATOM | 15155 | CE2 | TYR | G | 211 | −6.399 | 45.350 | 99.091 | 1.00 | 62.38 | C |
| ATOM | 15157 | CD2 | TYR | G | 211 | −7.380 | 45.528 | 98.125 | 1.00 | 47.34 | C |
| ATOM | 15159 | C | TYR | G | 211 | −9.349 | 44.906 | 93.726 | 1.00 | 45.54 | C |
| ATOM | 15160 | O | TYR | G | 211 | −9.278 | 43.822 | 93.096 | 1.00 | 39.16 | O |
| ATOM | 15161 | N | LEU | G | 212 | −10.263 | 45.861 | 93.483 | 1.00 | 32.73 | N |
| ATOM | 15163 | CA | LEU | G | 212 | −11.335 | 45.631 | 92.551 | 1.00 | 33.94 | C |
| ATOM | 15165 | CB | LEU | G | 212 | −12.465 | 46.625 | 92.761 | 1.00 | 43.90 | C |
| ATOM | 15168 | CG | LEU | G | 212 | −13.374 | 46.507 | 93.990 | 1.00 | 45.99 | C |
| ATOM | 15170 | CD1 | LEU | G | 212 | −14.355 | 47.671 | 93.912 | 1.00 | 44.51 | C |
| ATOM | 15174 | CD2 | LEU | G | 212 | −14.096 | 45.167 | 94.000 | 1.00 | 62.96 | C |
| ATOM | 15178 | C | LEU | G | 212 | −10.774 | 45.767 | 91.140 | 1.00 | 41.77 | C |
| ATOM | 15179 | O | LEU | G | 212 | −11.249 | 45.099 | 90.203 | 1.00 | 45.86 | O |
| ATOM | 15180 | N | ASN | G | 213 | −9.752 | 46.607 | 90.983 | 1.00 | 46.18 | N |
| ATOM | 15182 | CA | ASN | G | 213 | −9.101 | 46.765 | 89.670 | 1.00 | 44.55 | C |
| ATOM | 15184 | CB | ASN | G | 213 | −8.410 | 48.113 | 89.479 | 1.00 | 33.88 | C |
| ATOM | 15187 | CG | ASN | G | 213 | −9.383 | 49.245 | 89.336 | 1.00 | 44.48 | C |
| ATOM | 15188 | OD1 | ASN | G | 213 | −10.389 | 49.131 | 88.625 | 1.00 | 59.73 | O |
| ATOM | 15189 | ND2 | ASN | G | 213 | −9.082 | 50.364 | 89.992 | 1.00 | 36.61 | N |
| ATOM | 15192 | C | ASN | G | 213 | −8.085 | 45.668 | 89.435 | 1.00 | 43.42 | C |
| ATOM | 15193 | O | ASN | G | 213 | −7.606 | 45.497 | 88.320 | 1.00 | 48.13 | O |
| ATOM | 15194 | N | PHE | G | 214 | −7.808 | 44.862 | 90.445 | 1.00 | 45.75 | N |
| ATOM | 15196 | CA | PHE | G | 214 | −7.298 | 43.522 | 90.142 | 1.00 | 53.59 | C |
| ATOM | 15198 | CB | PHE | G | 214 | −6.650 | 42.872 | 91.370 | 1.00 | 52.47 | C |
| ATOM | 15201 | CG | PHE | G | 214 | −5.247 | 43.317 | 91.613 | 1.00 | 45.56 | C |
| ATOM | 15202 | CD1 | PHE | G | 214 | −4.218 | 42.898 | 90.766 | 1.00 | 68.06 | C |
| ATOM | 15204 | CE1 | PHE | G | 214 | −2.905 | 43.287 | 90.984 | 1.00 | 68.07 | C |
| ATOM | 15206 | CZ | PHE | G | 214 | −2.605 | 44.140 | 92.052 | 1.00 | 67.67 | C |
| ATOM | 15208 | CE2 | PHE | G | 214 | −3.619 | 44.565 | 92.904 | 1.00 | 60.46 | C |
| ATOM | 15210 | CD2 | PHE | G | 214 | −4.940 | 44.151 | 92.679 | 1.00 | 59.80 | C |
| ATOM | 15212 | C | PHE | G | 214 | −8.331 | 42.573 | 89.522 | 1.00 | 51.64 | C |
| ATOM | 15213 | O | PHE | G | 214 | −8.022 | 41.910 | 88.541 | 1.00 | 44.07 | O |
| ATOM | 15214 | N | PHE | G | 215 | −9.529 | 42.496 | 90.108 | 1.00 | 52.42 | N |
| ATOM | 15216 | CA | PHE | G | 215 | −10.571 | 41.575 | 89.646 | 1.00 | 51.71 | C |
| ATOM | 15218 | CB | PHE | G | 215 | −11.811 | 41.689 | 90.538 | 1.00 | 57.99 | C |
| ATOM | 15221 | CG | PHE | G | 215 | −11.561 | 41.440 | 91.980 | 1.00 | 51.31 | C |
| ATOM | 15222 | CD1 | PHE | G | 215 | −10.447 | 40.777 | 92.393 | 1.00 | 58.45 | C |
| ATOM | 15224 | CE1 | PHE | G | 215 | −10.228 | 40.549 | 93.734 | 1.00 | 58.50 | C |
| ATOM | 15226 | CZ | PHE | G | 215 | −11.141 | 40.946 | 94.661 | 1.00 | 40.38 | C |
| ATOM | 15228 | CE2 | PHE | G | 215 | −12.220 | 41.649 | 94.259 | 1.00 | 45.14 | C |
| ATOM | 15230 | CD2 | PHE | G | 215 | −12.431 | 41.904 | 92.925 | 1.00 | 46.15 | C |
| ATOM | 15232 | C | PHE | G | 215 | −11.058 | 41.893 | 88.221 | 1.00 | 50.32 | C |
| ATOM | 15233 | O | PHE | G | 215 | −11.487 | 40.981 | 87.493 | 1.00 | 35.25 | O |
| ATOM | 15234 | N | THR | G | 216 | −11.115 | 43.180 | 87.861 | 1.00 | 39.21 | N |
| ATOM | 15236 | CA | THR | G | 216 | −11.633 | 43.488 | 86.539 | 1.00 | 44.06 | C |
| ATOM | 15238 | CB | THR | G | 216 | −11.969 | 44.959 | 86.390 | 1.00 | 41.02 | C |
| ATOM | 15240 | OG1 | THR | G | 216 | −10.858 | 45.756 | 86.760 | 1.00 | 35.72 | O |
| ATOM | 15242 | CG2 | THR | G | 216 | −13.001 | 45.348 | 87.408 | 1.00 | 62.45 | C |
| ATOM | 15246 | C | THR | G | 216 | −10.755 | 42.986 | 85.397 | 1.00 | 41.79 | C |
| ATOM | 15247 | O | THR | G | 216 | −11.138 | 43.048 | 84.241 | 1.00 | 45.15 | O |
| ATOM | 15248 | N | LYS | G | 217 | −9.619 | 42.403 | 85.747 | 1.00 | 44.95 | N |
| ATOM | 15250 | CA | LYS | G | 217 | −8.712 | 41.760 | 84.810 | 1.00 | 40.42 | C |
| ATOM | 15252 | CB | LYS | G | 217 | −7.442 | 41.423 | 85.594 | 1.00 | 47.40 | C |
| ATOM | 15255 | CG | LYS | G | 217 | −6.143 | 42.207 | 85.240 | 1.00 | 61.19 | C |
| ATOM | 15258 | CD | LYS | G | 217 | −5.937 | 43.570 | 85.892 | 1.00 | 76.90 | C |
| ATOM | 15261 | CE | LYS | G | 217 | −4.480 | 44.016 | 85.780 | 1.00 | 86.22 | C |
| ATOM | 15264 | NZ | LYS | G | 217 | −3.655 | 43.477 | 86.924 | 1.00 | 89.38 | N |
| ATOM | 15268 | C | LYS | G | 217 | −9.342 | 40.488 | 84.244 | 1.00 | 43.06 | C |
| ATOM | 15269 | O | LYS | G | 217 | −8.871 | 39.915 | 83.270 | 1.00 | 45.87 | O |
| ATOM | 15270 | N | ALA | G | 218 | −10.459 | 40.082 | 84.839 | 1.00 | 47.53 | N |
| ATOM | 15272 | CA | ALA | G | 218 | −11.273 | 38.966 | 84.351 | 1.00 | 40.19 | C |
| ATOM | 15274 | CB | ALA | G | 218 | −11.959 | 38.325 | 85.528 | 1.00 | 36.28 | C |
| ATOM | 15278 | C | ALA | G | 218 | −12.321 | 39.336 | 83.279 | 1.00 | 42.74 | C |
| ATOM | 15279 | O | ALA | G | 218 | −12.948 | 38.431 | 82.705 | 1.00 | 39.84 | O |
| ATOM | 15280 | N | THR | G | 219 | −12.484 | 40.639 | 83.019 | 1.00 | 35.84 | N |
| ATOM | 15282 | CA | THR | G | 219 | −13.441 | 41.175 | 82.059 | 1.00 | 30.47 | C |
| ATOM | 15284 | CB | THR | G | 219 | −13.186 | 42.667 | 81.871 | 1.00 | 35.62 | C |
| ATOM | 15286 | OG1 | THR | G | 219 | −13.277 | 43.342 | 83.131 | 1.00 | 38.54 | O |
| ATOM | 15288 | CG2 | THR | G | 219 | −14.269 | 43.313 | 81.034 | 1.00 | 20.81 | C |
| ATOM | 15292 | C | THR | G | 219 | −13.494 | 40.470 | 80.704 | 1.00 | 34.59 | C |
| ATOM | 15293 | O | THR | G | 219 | −14.561 | 40.077 | 80.253 | 1.00 | 33.56 | O |
| ATOM | 15294 | N | PRO | G | 220 | −12.348 | 40.151 | 80.113 | 1.00 | 46.00 | N |
| ATOM | 15295 | CA | PRO | G | 220 | −12.356 | 39.407 | 78.848 | 1.00 | 46.28 | C |
| ATOM | 15297 | CB | PRO | G | 220 | −10.884 | 39.037 | 78.641 | 1.00 | 42.42 | C |
| ATOM | 15300 | CG | PRO | G | 220 | −10.122 | 39.901 | 79.523 | 1.00 | 41.05 | C |
| ATOM | 15303 | CD | PRO | G | 220 | −10.996 | 40.215 | 80.702 | 1.00 | 47.93 | C |
| ATOM | 15306 | C | PRO | G | 220 | −13.199 | 38.120 | 78.934 | 1.00 | 39.72 | C |
| ATOM | 15307 | O | PRO | G | 220 | −13.672 | 37.595 | 77.940 | 1.00 | 43.96 | O |
| ATOM | 15308 | N | LEU | G | 221 | −13.344 | 37.577 | 80.126 | 1.00 | 36.74 | N |
| ATOM | 15310 | CA | LEU | G | 221 | −13.837 | 36.222 | 80.257 | 1.00 | 42.19 | C |
| ATOM | 15312 | CB | LEU | G | 221 | −13.583 | 35.745 | 81.693 | 1.00 | 41.78 | C |
| ATOM | 15315 | CG | LEU | G | 221 | −12.182 | 35.193 | 81.876 | 1.00 | 48.49 | C |
| ATOM | 15317 | CD1 | LEU | G | 221 | −11.803 | 34.922 | 83.351 | 1.00 | 40.84 | C |
| ATOM | 15321 | CD2 | LEU | G | 221 | −12.165 | 33.931 | 81.033 | 1.00 | 39.95 | C |
| ATOM | 15325 | C | LEU | G | 221 | −15.346 | 36.216 | 79.963 | 1.00 | 44.52 | C |
| ATOM | 15326 | O | LEU | G | 221 | −15.920 | 35.167 | 79.640 | 1.00 | 44.39 | O |
| ATOM | 15327 | N | SER | G | 222 | −15.984 | 37.381 | 80.102 | 1.00 | 38.04 | N |
| ATOM | 15329 | CA | SER | G | 222 | −17.433 | 37.450 | 80.078 | 1.00 | 37.67 | C |
| ATOM | 15331 | CB | SER | G | 222 | −18.019 | 36.874 | 81.363 | 1.00 | 47.47 | C |
| ATOM | 15334 | OG | SER | G | 222 | −19.397 | 37.197 | 81.484 | 1.00 | 48.64 | O |
| ATOM | 15336 | C | SER | G | 222 | −17.883 | 38.887 | 79.957 | 1.00 | 40.27 | C |
| ATOM | 15337 | O | SER | G | 222 | −17.302 | 39.793 | 80.534 | 1.00 | 42.46 | O |
| ATOM | 15338 | N | SER | G | 223 | −18.967 | 39.075 | 79.228 | 1.00 | 43.69 | N |
| ATOM | 15340 | CA | SER | G | 223 | −19.501 | 40.388 | 78.974 | 1.00 | 40.84 | C |
| ATOM | 15342 | CB | SER | G | 223 | −20.347 | 40.340 | 77.710 | 1.00 | 47.16 | C |
| ATOM | 15345 | OG | SER | G | 223 | −21.390 | 39.378 | 77.836 | 1.00 | 64.77 | O |
| ATOM | 15347 | C | SER | G | 223 | −20.372 | 40.869 | 80.128 | 1.00 | 43.56 | C |
| ATOM | 15348 | O | SER | G | 223 | −20.667 | 42.055 | 80.203 | 1.00 | 49.98 | O |
| ATOM | 15349 | N | THR | G | 224 | −20.802 | 39.991 | 81.027 | 1.00 | 39.90 | N |
| ATOM | 15351 | CA | THR | G | 224 | −21.337 | 40.482 | 82.288 | 1.00 | 48.27 | C |
| ATOM | 15353 | CB | THR | G | 224 | −22.835 | 40.192 | 82.480 | 1.00 | 49.94 | C |
| ATOM | 15355 | OG1 | THR | G | 224 | −23.051 | 38.782 | 82.454 | 1.00 | 45.61 | O |
| ATOM | 15357 | CG2 | THR | G | 224 | −23.699 | 40.835 | 81.346 | 1.00 | 57.55 | C |
| ATOM | 15361 | C | THR | G | 224 | −20.588 | 39.960 | 83.493 | 1.00 | 50.19 | C |
| ATOM | 15362 | O | THR | G | 224 | −20.095 | 38.825 | 83.478 | 1.00 | 47.76 | O |
| ATOM | 15363 | N | VAL | G | 225 | −20.530 | 40.835 | 84.503 | 1.00 | 45.94 | N |
| ATOM | 15365 | CA | VAL | G | 225 | −20.232 | 40.513 | 85.886 | 1.00 | 37.22 | C |
| ATOM | 15367 | CB | VAL | G | 225 | −19.155 | 41.474 | 86.392 | 1.00 | 40.96 | C |
| ATOM | 15369 | CG1 | VAL | G | 225 | −19.683 | 42.912 | 86.511 | 1.00 | 25.69 | C |
| ATOM | 15373 | CG2 | VAL | G | 225 | −18.604 | 40.960 | 87.703 | 1.00 | 53.92 | C |
| ATOM | 15377 | C | VAL | G | 225 | −21.440 | 40.685 | 86.804 | 1.00 | 41.77 | C |
| ATOM | 15378 | O | VAL | G | 225 | −22.386 | 41.420 | 86.504 | 1.00 | 51.01 | O |
| ATOM | 15379 | N | THR | G | 226 | −21.406 | 40.032 | 87.956 | 1.00 | 47.85 | N |
| ATOM | 15381 | CA | THR | G | 226 | −22.504 | 40.151 | 88.903 | 1.00 | 48.40 | C |
| ATOM | 15383 | CB | THR | G | 226 | −23.406 | 38.882 | 88.866 | 1.00 | 53.74 | C |
| ATOM | 15385 | OG1 | THR | G | 226 | −23.400 | 38.256 | 90.156 | 1.00 | 57.44 | O |
| ATOM | 15387 | CG2 | THR | G | 226 | −22.869 | 37.804 | 87.918 | 1.00 | 49.62 | C |
| ATOM | 15391 | C | THR | G | 226 | −21.961 | 40.325 | 90.307 | 1.00 | 44.35 | C |
| ATOM | 15392 | O | THR | G | 226 | −21.235 | 39.464 | 90.796 | 1.00 | 35.46 | O |
| ATOM | 15393 | N | LEU | G | 227 | −22.343 | 41.421 | 90.953 | 1.00 | 44.89 | N |
| ATOM | 15395 | CA | LEU | G | 227 | −21.907 | 41.687 | 92.315 | 1.00 | 42.52 | C |
| ATOM | 15397 | CB | LEU | G | 227 | −21.638 | 43.159 | 92.535 | 1.00 | 43.82 | C |
| ATOM | 15400 | CG | LEU | G | 227 | −21.389 | 43.962 | 91.274 | 1.00 | 53.66 | C |
| ATOM | 15402 | CD1 | LEU | G | 227 | −21.274 | 45.428 | 91.599 | 1.00 | 54.45 | C |
| ATOM | 15406 | CD2 | LEU | G | 227 | −20.084 | 43.444 | 90.691 | 1.00 | 68.13 | C |
| ATOM | 15410 | C | LEU | G | 227 | −22.976 | 41.318 | 93.292 | 1.00 | 43.80 | C |
| ATOM | 15411 | O | LEU | G | 227 | −24.128 | 41.730 | 93.206 | 1.00 | 45.20 | O |
| ATOM | 15412 | N | SER | G | 228 | −22.522 | 40.653 | 94.328 | 1.00 | 46.54 | N |
| ATOM | 15414 | CA | SER | G | 228 | −23.440 | 40.164 | 95.313 | 1.00 | 52.19 | C |
| ATOM | 15416 | CB | SER | G | 228 | −23.739 | 38.674 | 94.971 | 1.00 | 57.04 | C |
| ATOM | 15419 | OG | SER | G | 228 | −23.281 | 37.712 | 95.908 | 1.00 | 59.42 | O |
| ATOM | 15421 | C | SER | G | 228 | −22.854 | 40.505 | 96.707 | 1.00 | 49.70 | C |
| ATOM | 15422 | O | SER | G | 228 | −21.674 | 40.265 | 96.949 | 1.00 | 51.84 | O |
| ATOM | 15423 | N | MET | G | 229 | −23.655 | 41.135 | 97.575 | 1.00 | 49.38 | N |
| ATOM | 15425 | CA | MET | G | 229 | −23.191 | 41.851 | 98.757 | 1.00 | 51.29 | C |
| ATOM | 15427 | CB | MET | G | 229 | −22.786 | 43.267 | 98.369 | 1.00 | 58.33 | C |
| ATOM | 15430 | CG | MET | G | 229 | −23.680 | 43.893 | 97.299 | 1.00 | 58.53 | C |
| ATOM | 15433 | SD | MET | G | 229 | −22.827 | 45.335 | 96.681 | 1.00 | 60.11 | S |
| ATOM | 15434 | CE | MET | G | 229 | −24.045 | 45.999 | 95.518 | 1.00 | 49.78 | C |
| ATOM | 15438 | C | MET | G | 229 | −24.253 | 41.944 | 99.861 | 1.00 | 55.90 | C |
| ATOM | 15439 | O | MET | G | 229 | −25.390 | 42.364 | 99.633 | 1.00 | 51.62 | O |
| ATOM | 15440 | N | SER | G | 230 | −23.829 | 41.556 | 101.056 | 1.00 | 53.85 | N |
| ATOM | 15442 | CA | SER | G | 230 | −24.483 | 41.850 | 102.314 | 1.00 | 57.15 | C |
| ATOM | 15444 | CB | SER | G | 230 | −24.809 | 40.534 | 103.037 | 1.00 | 56.49 | C |
| ATOM | 15447 | OG | SER | G | 230 | −25.806 | 39.802 | 102.357 | 1.00 | 74.49 | O |
| ATOM | 15449 | C | SER | G | 230 | −23.496 | 42.596 | 103.220 | 1.00 | 60.05 | C |
| ATOM | 15450 | O | SER | G | 230 | −22.329 | 42.196 | 103.372 | 1.00 | 57.21 | O |
| ATOM | 15451 | N | ALA | G | 231 | −24.005 | 43.612 | 103.906 | 1.00 | 59.82 | N |
| ATOM | 15453 | CA | ALA | G | 231 | −23.431 | 44.112 | 105.147 | 1.00 | 54.28 | C |
| ATOM | 15455 | CB | ALA | G | 231 | −24.529 | 44.666 | 105.961 | 1.00 | 62.41 | C |
| ATOM | 15459 | C | ALA | G | 231 | −22.659 | 43.100 | 105.970 | 1.00 | 57.76 | C |
| ATOM | 15460 | O | ALA | G | 231 | −23.157 | 42.035 | 106.276 | 1.00 | 64.52 | O |
| ATOM | 15461 | N | ASP | G | 232 | −21.423 | 43.439 | 106.299 | 1.00 | 62.92 | N |
| ATOM | 15463 | CA | ASP | G | 232 | −20.603 | 42.723 | 107.268 | 1.00 | 68.86 | C |
| ATOM | 15465 | CB | ASP | G | 232 | −21.230 | 42.749 | 108.685 | 1.00 | 74.20 | C |
| ATOM | 15468 | CG | ASP | G | 232 | −21.103 | 44.136 | 109.382 | 1.00 | 82.92 | C |
| ATOM | 15469 | OD1 | ASP | G | 232 | −19.966 | 44.618 | 109.564 | 1.00 | 93.31 | O |
| ATOM | 15470 | OD2 | ASP | G | 232 | −22.062 | 44.858 | 109.747 | 1.00 | 74.89 | O |
| ATOM | 15471 | C | ASP | G | 232 | −20.098 | 41.348 | 106.787 | 1.00 | 70.10 | C |
| ATOM | 15472 | O | ASP | G | 232 | −19.446 | 40.606 | 107.531 | 1.00 | 69.76 | O |
| ATOM | 15473 | N | VAL | G | 233 | −20.263 | 41.079 | 105.493 | 1.00 | 71.87 | N |
| ATOM | 15475 | CA | VAL | G | 233 | −19.483 | 40.038 | 104.797 | 1.00 | 65.32 | C |
| ATOM | 15477 | CB | VAL | G | 233 | −20.354 | 38.825 | 104.450 | 1.00 | 60.78 | C |
| ATOM | 15479 | CG1 | VAL | G | 233 | −21.060 | 38.354 | 105.695 | 1.00 | 74.83 | C |
| ATOM | 15483 | CG2 | VAL | G | 233 | −21.351 | 39.150 | 103.358 | 1.00 | 47.21 | C |
| ATOM | 15487 | C | VAL | G | 233 | −18.801 | 40.469 | 103.494 | 1.00 | 61.83 | C |
| ATOM | 15488 | O | VAL | G | 233 | −19.135 | 41.517 | 102.902 | 1.00 | 62.44 | O |
| ATOM | 15489 | N | PRO | G | 234 | −17.826 | 39.659 | 103.086 | 1.00 | 52.15 | N |
| ATOM | 15490 | CA | PRO | G | 234 | −17.080 | 39.896 | 101.848 | 1.00 | 49.48 | C |
| ATOM | 15492 | CB | PRO | G | 234 | −16.184 | 38.672 | 101.693 | 1.00 | 54.28 | C |
| ATOM | 15495 | CG | PRO | G | 234 | −16.497 | 37.739 | 102.852 | 1.00 | 56.71 | C |
| ATOM | 15498 | CD | PRO | G | 234 | −17.308 | 38.509 | 103.845 | 1.00 | 51.65 | C |
| ATOM | 15501 | C | PRO | G | 234 | −18.023 | 39.945 | 100.695 | 1.00 | 44.49 | C |
| ATOM | 15502 | O | PRO | G | 234 | −19.039 | 39.252 | 100.725 | 1.00 | 47.26 | O |
| ATOM | 15503 | N | LEU | G | 235 | −17.671 | 40.801 | 99.742 | 1.00 | 50.77 | N |
| ATOM | 15505 | CA | LEU | G | 235 | −18.330 | 40.998 | 98.442 | 1.00 | 47.80 | C |
| ATOM | 15507 | CB | LEU | G | 235 | −17.926 | 42.355 | 97.852 | 1.00 | 43.53 | C |
| ATOM | 15510 | CG | LEU | G | 235 | −18.238 | 42.664 | 96.390 | 1.00 | 34.64 | C |
| ATOM | 15512 | CD1 | LEU | G | 235 | −19.645 | 43.149 | 96.277 | 1.00 | 45.74 | C |
| ATOM | 15516 | CD2 | LEU | G | 235 | −17.398 | 43.804 | 95.873 | 1.00 | 37.47 | C |
| ATOM | 15520 | C | LEU | G | 235 | −17.855 | 39.948 | 97.469 | 1.00 | 44.24 | C |
| ATOM | 15521 | O | LEU | G | 235 | −16.756 | 39.411 | 97.572 | 1.00 | 50.35 | O |
| ATOM | 15522 | N | VAL | G | 236 | −18.710 | 39.639 | 96.520 | 1.00 | 51.44 | N |
| ATOM | 15524 | CA | VAL | G | 236 | −18.367 | 38.637 | 95.534 | 1.00 | 50.05 | C |
| ATOM | 15526 | CB | VAL | G | 236 | −19.275 | 37.418 | 95.611 | 1.00 | 48.01 | C |
| ATOM | 15528 | CG1 | VAL | G | 236 | −18.752 | 36.402 | 94.653 | 1.00 | 60.40 | C |
| ATOM | 15532 | CG2 | VAL | G | 236 | −19.272 | 36.829 | 97.051 | 1.00 | 44.48 | C |
| ATOM | 15536 | C | VAL | G | 236 | −18.518 | 39.339 | 94.219 | 1.00 | 47.84 | C |
| ATOM | 15537 | O | VAL | G | 236 | −19.522 | 40.012 | 93.993 | 1.00 | 44.97 | O |
| ATOM | 15538 | N | VAL | G | 237 | −17.452 | 39.269 | 93.424 | 1.00 | 52.22 | N |
| ATOM | 15540 | CA | VAL | G | 237 | −17.498 | 39.590 | 92.002 | 1.00 | 47.42 | C |
| ATOM | 15542 | CB | VAL | G | 237 | −16.425 | 40.603 | 91.687 | 1.00 | 45.17 | C |
| ATOM | 15544 | CG1 | VAL | G | 237 | −16.897 | 41.581 | 90.649 | 1.00 | 45.48 | C |
| ATOM | 15548 | CG2 | VAL | G | 237 | −16.090 | 41.368 | 92.932 | 1.00 | 38.83 | C |
| ATOM | 15552 | C | VAL | G | 237 | −17.325 | 38.321 | 91.180 | 1.00 | 43.64 | C |
| ATOM | 15553 | O | VAL | G | 237 | −16.326 | 37.634 | 91.296 | 1.00 | 40.81 | O |
| ATOM | 15554 | N | GLU | G | 238 | −18.355 | 37.980 | 90.411 | 1.00 | 55.46 | N |
| ATOM | 15556 | CA | GLU | G | 238 | −18.471 | 36.690 | 89.701 | 1.00 | 54.43 | C |
| ATOM | 15558 | CB | GLU | G | 238 | −19.758 | 35.952 | 90.115 | 1.00 | 58.80 | C |
| ATOM | 15561 | CG | GLU | G | 238 | −19.824 | 34.489 | 89.682 | 1.00 | 65.15 | C |
| ATOM | 15564 | CD | GLU | G | 238 | −21.082 | 33.764 | 90.119 | 1.00 | 79.90 | C |
| ATOM | 15565 | OE1 | GLU | G | 238 | −21.866 | 34.296 | 90.941 | 1.00 | 89.20 | O |
| ATOM | 15566 | OE2 | GLU | G | 238 | −21.267 | 32.632 | 89.626 | 1.00 | 102.65 | O |
| ATOM | 15567 | C | GLU | G | 238 | −18.459 | 36.897 | 88.170 | 1.00 | 49.96 | C |
| ATOM | 15568 | O | GLU | G | 238 | −19.241 | 37.684 | 87.603 | 1.00 | 36.08 | O |
| ATOM | 15569 | N | TYR | G | 239 | −17.505 | 36.229 | 87.527 | 1.00 | 42.77 | N |
| ATOM | 15571 | CA | TYR | G | 239 | −17.446 | 36.174 | 86.078 | 1.00 | 39.12 | C |
| ATOM | 15573 | CB | TYR | G | 239 | −16.101 | 36.694 | 85.595 | 1.00 | 41.34 | C |
| ATOM | 15576 | CG | TYR | G | 239 | −15.776 | 38.129 | 85.979 | 1.00 | 41.48 | C |
| ATOM | 15577 | CD1 | TYR | G | 239 | −15.172 | 38.439 | 87.182 | 1.00 | 32.71 | C |
| ATOM | 15579 | CE1 | TYR | G | 239 | −14.850 | 39.735 | 87.501 | 1.00 | 37.23 | C |
| ATOM | 15581 | CZ | TYR | G | 239 | −15.082 | 40.740 | 86.591 | 1.00 | 33.71 | C |
| ATOM | 15582 | OH | TYR | G | 239 | −14.790 | 42.066 | 86.884 | 1.00 | 44.24 | O |
| ATOM | 15584 | CE2 | TYR | G | 239 | −15.595 | 40.430 | 85.366 | 1.00 | 26.03 | C |
| ATOM | 15586 | CD2 | TYR | G | 239 | −15.957 | 39.152 | 85.074 | 1.00 | 33.13 | C |
| ATOM | 15588 | C | TYR | G | 239 | −17.649 | 34.729 | 85.632 | 1.00 | 40.05 | C |
| ATOM | 15589 | O | TYR | G | 239 | −16.969 | 33.791 | 86.063 | 1.00 | 41.94 | O |
| ATOM | 15590 | N | LYS | G | 240 | −18.629 | 34.524 | 84.773 | 1.00 | 49.38 | N |
| ATOM | 15592 | CA | LYS | G | 240 | −18.924 | 33.164 | 84.379 | 1.00 | 51.35 | C |
| ATOM | 15594 | CB | LYS | G | 240 | −20.445 | 32.909 | 84.211 | 1.00 | 56.31 | C |
| ATOM | 15597 | CG | LYS | G | 240 | −21.184 | 32.712 | 85.632 | 1.00 | 75.08 | C |
| ATOM | 15600 | CD | LYS | G | 240 | −22.435 | 31.763 | 85.721 | 1.00 | 86.90 | C |
| ATOM | 15603 | CE | LYS | G | 240 | −22.239 | 30.467 | 86.592 | 1.00 | 90.71 | C |
| ATOM | 15606 | NZ | LYS | G | 240 | −23.027 | 29.220 | 86.185 | 1.00 | 52.66 | N |
| ATOM | 15610 | C | LYS | G | 240 | −18.059 | 32.890 | 83.162 | 1.00 | 44.74 | C |
| ATOM | 15611 | O | LYS | G | 240 | −18.036 | 33.662 | 82.230 | 1.00 | 42.22 | O |
| ATOM | 15612 | N | ILE | G | 241 | −17.248 | 31.846 | 83.257 | 1.00 | 52.13 | N |
| ATOM | 15614 | CA | ILE | G | 241 | −16.465 | 31.301 | 82.141 | 1.00 | 57.94 | C |
| ATOM | 15616 | CB | ILE | G | 241 | −15.223 | 30.515 | 82.667 | 1.00 | 58.08 | C |
| ATOM | 15618 | CG1 | ILE | G | 241 | −14.309 | 31.407 | 83.506 | 1.00 | 46.26 | C |
| ATOM | 15621 | CD1 | ILE | G | 241 | −13.597 | 30.672 | 84.595 | 1.00 | 54.11 | C |
| ATOM | 15625 | CG2 | ILE | G | 241 | −14.462 | 29.919 | 81.515 | 1.00 | 58.56 | C |
| ATOM | 15629 | C | ILE | G | 241 | −17.297 | 30.337 | 81.303 | 1.00 | 60.30 | C |
| ATOM | 15630 | O | ILE | G | 241 | −17.385 | 29.134 | 81.601 | 1.00 | 51.04 | O |
| ATOM | 15631 | N | ALA | G | 242 | −17.866 | 30.876 | 80.231 | 1.00 | 64.48 | N |
| ATOM | 15633 | CA | ALA | G | 242 | −19.205 | 30.498 | 79.841 | 1.00 | 65.73 | C |
| ATOM | 15635 | CB | ALA | G | 242 | −19.683 | 31.373 | 78.740 | 1.00 | 75.82 | C |
| ATOM | 15639 | C | ALA | G | 242 | −19.152 | 29.072 | 79.378 | 1.00 | 65.45 | C |
| ATOM | 15640 | O | ALA | G | 242 | −18.246 | 28.699 | 78.646 | 1.00 | 60.75 | O |
| ATOM | 15641 | N | ASP | G | 243 | −20.107 | 28.288 | 79.857 | 1.00 | 72.20 | N |
| ATOM | 15643 | CA | ASP | G | 243 | −20.130 | 26.844 | 79.663 | 1.00 | 79.01 | C |
| ATOM | 15645 | CB | ASP | G | 243 | −20.637 | 26.502 | 78.257 | 1.00 | 84.80 | C |
| ATOM | 15648 | CG | ASP | G | 243 | −22.144 | 26.759 | 78.106 | 1.00 | 107.21 | C |
| ATOM | 15649 | OD1 | ASP | G | 243 | −22.702 | 26.443 | 77.027 | 1.00 | 116.29 | O |
| ATOM | 15650 | OD2 | ASP | G | 243 | −22.842 | 27.273 | 79.020 | 1.00 | 116.34 | O |
| ATOM | 15651 | C | ASP | G | 243 | −18.777 | 26.206 | 79.912 | 1.00 | 75.40 | C |
| ATOM | 15652 | O | ASP | G | 243 | −18.231 | 25.541 | 79.027 | 1.00 | 76.63 | O |
| ATOM | 15653 | N | MET | G | 244 | −18.277 | 26.404 | 81.131 | 1.00 | 65.49 | N |
| ATOM | 15655 | CA | MET | G | 244 | −17.224 | 25.585 | 81.734 | 1.00 | 56.48 | C |
| ATOM | 15657 | CB | MET | G | 244 | −15.875 | 26.126 | 81.296 | 1.00 | 54.90 | C |
| ATOM | 15660 | CG | MET | G | 244 | −15.544 | 25.842 | 79.891 | 1.00 | 53.04 | C |
| ATOM | 15663 | SD | MET | G | 244 | −13.802 | 25.754 | 79.767 | 1.00 | 56.97 | S |
| ATOM | 15664 | CE | MET | G | 244 | −13.484 | 24.226 | 80.771 | 1.00 | 69.54 | C |
| ATOM | 15668 | C | MET | G | 244 | −17.239 | 25.728 | 83.265 | 1.00 | 54.43 | C |
| ATOM | 15669 | O | MET | G | 244 | −16.847 | 24.811 | 83.995 | 1.00 | 47.17 | O |
| ATOM | 15670 | N | GLY | G | 245 | −17.575 | 26.932 | 83.735 | 1.00 | 42.14 | N |
| ATOM | 15672 | CA | GLY | G | 245 | −17.475 | 27.267 | 85.137 | 1.00 | 41.90 | C |
| ATOM | 15675 | C | GLY | G | 245 | −17.433 | 28.759 | 85.399 | 1.00 | 47.39 | C |
| ATOM | 15676 | O | GLY | G | 245 | −18.037 | 29.547 | 84.667 | 1.00 | 39.16 | O |
| ATOM | 15677 | N | HIS | G | 246 | −16.718 | 29.145 | 86.455 | 1.00 | 52.90 | N |
| ATOM | 15679 | CA | HIS | G | 246 | −16.709 | 30.538 | 86.875 | 1.00 | 50.96 | C |
| ATOM | 15681 | CB | HIS | G | 246 | −17.873 | 30.783 | 87.819 | 1.00 | 54.53 | C |
| ATOM | 15684 | CG | HIS | G | 246 | −17.998 | 29.752 | 88.896 | 1.00 | 59.70 | C |
| ATOM | 15685 | ND1 | HIS | G | 246 | −18.997 | 28.801 | 88.900 | 1.00 | 67.16 | N |
| ATOM | 15687 | CE1 | HIS | G | 246 | −18.873 | 28.051 | 89.979 | 1.00 | 83.37 | C |
| ATOM | 15689 | NE2 | HIS | G | 246 | −17.812 | 28.458 | 90.655 | 1.00 | 73.70 | N |
| ATOM | 15691 | CD2 | HIS | G | 246 | −17.255 | 29.532 | 90.007 | 1.00 | 50.88 | C |
| ATOM | 15693 | C | HIS | G | 246 | −15.437 | 30.972 | 87.578 | 1.00 | 51.64 | C |
| ATOM | 15694 | O | HIS | G | 246 | −14.648 | 30.172 | 88.122 | 1.00 | 47.08 | O |
| ATOM | 15695 | N | LEU | G | 247 | −15.302 | 32.290 | 87.646 | 1.00 | 48.53 | N |
| ATOM | 15697 | CA | LEU | G | 247 | −14.286 | 32.892 | 88.510 | 1.00 | 46.14 | C |
| ATOM | 15699 | CB | LEU | G | 247 | −13.289 | 33.695 | 87.648 | 1.00 | 50.52 | C |
| ATOM | 15702 | CG | LEU | G | 247 | −11.879 | 34.114 | 88.071 | 1.00 | 36.44 | C |
| ATOM | 15704 | CD1 | LEU | G | 247 | −11.055 | 33.042 | 88.800 | 1.00 | 45.65 | C |
| ATOM | 15708 | CD2 | LEU | G | 247 | −11.216 | 34.430 | 86.792 | 1.00 | 49.09 | C |
| ATOM | 15712 | C | LEU | G | 247 | −14.976 | 33.803 | 89.524 | 1.00 | 37.72 | C |
| ATOM | 15713 | O | LEU | G | 247 | −15.642 | 34.777 | 89.147 | 1.00 | 39.81 | O |
| ATOM | 15714 | N | LYS | G | 248 | −14.824 | 33.484 | 90.008 | 1.00 | 35.91 | N |
| ATOM | 15716 | CA | LYS | G | 248 | −15.431 | 34.290 | 91.860 | 1.00 | 32.99 | C |
| ATOM | 15718 | CB | LYS | G | 248 | −16.223 | 33.420 | 92.831 | 1.00 | 41.39 | C |
| ATOM | 15721 | CG | LYS | G | 248 | −17.477 | 32.750 | 92.286 | 1.00 | 42.97 | C |
| ATOM | 15724 | CD | LYS | G | 248 | −18.089 | 31.767 | 93.286 | 1.00 | 61.53 | C |
| ATOM | 15727 | CE | LYS | G | 248 | −19.477 | 31.278 | 92.841 | 1.00 | 64.96 | C |
| ATOM | 15730 | NZ | LYS | G | 248 | −20.224 | 30.941 | 94.081 | 1.00 | 71.44 | N |
| ATOM | 15734 | C | LYS | G | 248 | −14.303 | 34.916 | 92.638 | 1.00 | 37.56 | C |
| ATOM | 15735 | O | LYS | G | 248 | −13.490 | 34.187 | 93.233 | 1.00 | 36.88 | O |
| ATOM | 15736 | N | TYR | G | 249 | −14.258 | 36.247 | 92.602 | 1.00 | 35.86 | N |
| ATOM | 15738 | CA | TYR | G | 249 | −13.372 | 37.024 | 93.449 | 1.00 | 40.33 | C |
| ATOM | 15740 | CB | TYR | G | 249 | −12.969 | 38.293 | 92.702 | 1.00 | 48.70 | C |
| ATOM | 15743 | CG | TYR | G | 249 | −12.160 | 38.047 | 91.467 | 1.00 | 46.38 | C |
| ATOM | 15744 | CD1 | TYR | G | 249 | −10.804 | 37.788 | 91.545 | 1.00 | 48.13 | C |
| ATOM | 15746 | CE1 | TYR | G | 249 | −10.057 | 37.574 | 90.408 | 1.00 | 38.11 | C |
| ATOM | 15748 | CZ | TYR | G | 249 | −10.670 | 37.604 | 89.182 | 1.00 | 48.61 | C |
| ATOM | 15749 | OH | TYR | G | 249 | −9.943 | 37.383 | 88.046 | 1.00 | 56.55 | O |
| ATOM | 15751 | CE2 | TYR | G | 249 | −12.007 | 37.868 | 89.074 | 1.00 | 61.14 | C |
| ATOM | 15753 | CD2 | TYR | G | 249 | −12.745 | 38.089 | 90.219 | 1.00 | 63.96 | C |
| ATOM | 15755 | C | TYR | G | 249 | −14.090 | 37.461 | 94.704 | 1.00 | 38.14 | C |
| ATOM | 15756 | O | TYR | G | 249 | −15.007 | 38.276 | 94.630 | 1.00 | 45.30 | O |
| ATOM | 15757 | N | TYR | G | 250 | −13.594 | 37.046 | 95.858 | 1.00 | 37.46 | N |
| ATOM | 15759 | CA | TYR | G | 250 | −14.187 | 37.490 | 97.119 | 1.00 | 38.87 | C |
| ATOM | 15761 | CB | TYR | G | 250 | −14.288 | 36.329 | 98.107 | 1.00 | 41.77 | C |
| ATOM | 15764 | CG | TYR | G | 250 | −15.277 | 35.239 | 97.846 | 1.00 | 44.40 | C |
| ATOM | 15765 | CD1 | TYR | G | 250 | −14.998 | 34.219 | 96.952 | 1.00 | 51.61 | C |
| ATOM | 15767 | CE1 | TYR | G | 250 | −15.897 | 33.206 | 96.726 | 1.00 | 49.12 | C |
| ATOM | 15769 | CZ | TYR | G | 250 | −17.045 | 33.158 | 97.460 | 1.00 | 54.99 | C |
| ATOM | 15770 | OH | TYR | G | 250 | −17.948 | 32.153 | 97.264 | 1.00 | 72.01 | O |
| ATOM | 15772 | CE2 | TYR | G | 250 | −17.313 | 34.121 | 98.392 | 1.00 | 65.03 | C |
| ATOM | 15774 | CD2 | TYR | G | 250 | −16.432 | 35.153 | 98.587 | 1.00 | 61.22 | C |
| ATOM | 15776 | C | TYR | G | 250 | −13.314 | 38.554 | 97.798 | 1.00 | 40.01 | C |
| ATOM | 15777 | O | TYR | G | 250 | −12.103 | 38.381 | 97.962 | 1.00 | 42.59 | O |
| ATOM | 15778 | N | LEU | G | 251 | −13.970 | 39.560 | 98.356 | 1.00 | 44.89 | N |
| ATOM | 15780 | CA | LEU | G | 251 | −13.308 | 40.742 | 98.913 | 1.00 | 48.52 | C |
| ATOM | 15782 | CB | LEU | G | 251 | −13.565 | 41.946 | 97.989 | 1.00 | 49.72 | C |
| ATOM | 15785 | CG | LEU | G | 251 | −12.830 | 43.203 | 98.456 | 1.00 | 46.76 | C |
| ATOM | 15787 | CD1 | LEU | G | 251 | −11.427 | 42.805 | 98.887 | 1.00 | 43.55 | C |
| ATOM | 15791 | CD2 | LEU | G | 251 | −12.774 | 44.295 | 97.400 | 1.00 | 40.57 | C |
| ATOM | 15795 | C | LEU | G | 251 | −13.796 | 41.157 | 100.306 | 1.00 | 42.68 | C |
| ATOM | 15796 | O | LEU | G | 251 | −14.828 | 41.837 | 100.394 | 1.00 | 35.11 | O |
| ATOM | 15797 | N | ALA | G | 252 | −13.007 | 40.933 | 101.359 | 1.00 | 40.38 | N |
| ATOM | 15799 | CA | ALA | G | 252 | −13.256 | 41.656 | 102.635 | 1.00 | 49.87 | C |
| ATOM | 15801 | CB | ALA | G | 252 | −12.155 | 41.396 | 103.698 | 1.00 | 46.95 | C |
| ATOM | 15805 | C | ALA | G | 252 | −13.565 | 43.165 | 102.529 | 1.00 | 45.65 | C |
| ATOM | 15806 | O | ALA | G | 252 | −12.988 | 43.866 | 101.696 | 1.00 | 48.24 | O |
| ATOM | 15807 | N | PRO | G | 253 | −14.541 | 43.648 | 103.303 | 1.00 | 54.69 | N |
| ATOM | 15808 | CA | PRO | G | 253 | −14.906 | 45.075 | 103.284 | 1.00 | 55.18 | C |
| ATOM | 15810 | CB | PRO | G | 253 | −16.375 | 45.074 | 103.711 | 1.00 | 44.31 | C |
| ATOM | 15813 | CG | PRO | G | 253 | −16.404 | 43.960 | 104.746 | 1.00 | 51.23 | C |
| ATOM | 15816 | CD | PRO | G | 253 | −15.442 | 42.895 | 104.203 | 1.00 | 56.87 | C |
| ATOM | 15819 | C | PRO | G | 253 | −14.086 | 45.874 | 104.288 | 1.00 | 54.36 | C |
| ATOM | 15820 | O | PRO | G | 253 | −13.322 | 45.313 | 105.111 | 1.00 | 44.67 | O |
| ATOM | 15821 | N | LYS | G | 254 | −14.181 | 47.189 | 104.105 | 1.00 | 58.99 | N |
| ATOM | 15823 | CA | LYS | G | 254 | −13.515 | 48.155 | 104.968 | 1.00 | 68.79 | C |
| ATOM | 15825 | CB | LYS | G | 254 | −12.893 | 49.309 | 104.168 | 1.00 | 63.53 | C |
| ATOM | 15828 | CG | LYS | G | 254 | −11.457 | 49.035 | 103.690 | 1.00 | 74.91 | C |
| ATOM | 15831 | CD | LYS | G | 254 | −10.887 | 50.145 | 102.807 | 1.00 | 75.47 | C |
| ATOM | 15834 | CE | LYS | G | 254 | −10.489 | 51.336 | 103.672 | 1.00 | 85.20 | C |
| ATOM | 15837 | NZ | LYS | G | 254 | −10.026 | 52.539 | 102.925 | 1.00 | 83.61 | N |
| ATOM | 15841 | C | LYS | G | 254 | −14.641 | 48.632 | 105.852 | 1.00 | 73.52 | C |
| ATOM | 15842 | O | LYS | G | 254 | −15.530 | 49.337 | 105.384 | 1.00 | 77.94 | O |
| ATOM | 15843 | N | ILE | G | 255 | −14.708 | 48.109 | 107.069 | 1.00 | 81.29 | N |
| ATOM | 15845 | CA | ILE | G | 255 | −15.843 | 48.459 | 107.905 | 1.00 | 87.88 | C |
| ATOM | 15847 | CB | ILE | G | 255 | −16.528 | 47.209 | 108.547 | 1.00 | 82.07 | C |
| ATOM | 15849 | CG1 | ILE | G | 255 | −17.796 | 47.632 | 109.319 | 1.00 | 100.96 | C |
| ATOM | 15852 | CD1 | ILE | G | 255 | −18.772 | 48.654 | 108.613 | 1.00 | 99.92 | C |
| ATOM | 15856 | CG2 | ILE | G | 255 | −15.574 | 46.436 | 109.448 | 1.00 | 60.16 | C |
| ATOM | 15860 | C | ILE | G | 255 | −15.386 | 49.534 | 108.892 | 1.00 | 96.08 | C |
| ATOM | 15861 | O | ILE | G | 255 | −14.182 | 49.678 | 109.128 | 1.00 | 96.46 | O |
| ATOM | 15862 | N | GLU | G | 256 | −16.325 | 50.359 | 109.358 | 1.00 | 103.55 | N |
| ATOM | 15864 | CA | GLU | G | 256 | −16.006 | 51.456 | 110.273 | 1.00 | 108.14 | C |
| ATOM | 15866 | CB | GLU | G | 256 | −17.232 | 52.347 | 110.480 | 1.00 | 105.90 | C |
| ATOM | 15869 | CG | GLU | G | 256 | −18.231 | 51.760 | 111.460 | 1.00 | 109.94 | C |
| ATOM | 15872 | CD | GLU | G | 256 | −18.190 | 52.447 | 112.813 | 1.00 | 121.06 | C |
| ATOM | 15873 | OE1 | GLU | G | 256 | −18.676 | 51.851 | 113.798 | 1.00 | 125.79 | O |
| ATOM | 15874 | OE2 | GLU | G | 256 | −17.675 | 53.585 | 112.900 | 1.00 | 131.03 | O |
| ATOM | 15875 | C | GLU | G | 256 | −15.462 | 50.949 | 111.617 | 1.00 | 110.64 | C |
| ATOM | 15876 | O | GLU | G | 256 | −15.815 | 49.854 | 112.058 | 1.00 | 106.93 | O |
| ATOM | 15877 | N | ASP | G | 257 | −14.574 | 51.730 | 112.235 | 1.00 | 116.47 | N |
| ATOM | 15879 | CA | ASP | G | 257 | −13.877 | 51.322 | 113.454 | 1.00 | 121.75 | C |
| ATOM | 15881 | CB | ASP | G | 257 | −12.364 | 51.246 | 113.228 | 1.00 | 120.67 | C |
| ATOM | 15884 | CG | ASP | G | 257 | −11.628 | 50.733 | 114.453 | 1.00 | 119.72 | C |
| ATOM | 15885 | OD1 | ASP | G | 257 | −10.505 | 50.197 | 114.317 | 1.00 | 112.02 | O |
| ATOM | 15886 | OD2 | ASP | G | 257 | −12.126 | 50.796 | 115.598 | 1.00 | 111.67 | O |
| ATOM | 15887 | C | ASP | G | 257 | −14.123 | 52.312 | 114.585 | 1.00 | 129.33 | C |
| ATOM | 15888 | O | ASP | G | 257 | −13.474 | 53.363 | 114.630 | 1.00 | 132.03 | O |
| ATOM | 15889 | N | GLU | G | 258 | −15.029 | 51.960 | 115.500 | 1.00 | 135.72 | N |
| ATOM | 15891 | CA | GLU | G | 258 | −15.151 | 52.633 | 116.798 | 1.00 | 139.25 | C |
| ATOM | 15893 | CB | GLU | G | 258 | −16.327 | 53.619 | 116.770 | 1.00 | 138.72 | C |
| ATOM | 15896 | CG | GLU | G | 258 | −15.945 | 55.025 | 116.325 | 1.00 | 135.51 | C |
| ATOM | 15899 | CD | GLU | G | 258 | −15.185 | 55.792 | 117.391 | 1.00 | 127.37 | C |
| ATOM | 15900 | OE1 | GLU | G | 258 | −14.732 | 55.172 | 118.378 | 1.00 | 119.09 | O |
| ATOM | 15901 | OE2 | GLU | G | 258 | −15.047 | 57.024 | 117.242 | 1.00 | 121.28 | O |
| ATOM | 15902 | C | GLU | G | 258 | −15.296 | 51.643 | 117.969 | 1.00 | 141.87 | C |
| ATOM | 15903 | O | GLU | G | 258 | −14.985 | 50.453 | 117.819 | 1.00 | 142.87 | O |
| ATOM | 15904 | N | GLU | G | 259 | −15.753 | 52.142 | 119.124 | 1.00 | 143.64 | N |
| ATOM | 15906 | CA | GLU | G | 259 | −16.038 | 51.320 | 120.317 | 1.00 | 141.97 | C |
| ATOM | 15908 | CB | GLU | G | 259 | −16.318 | 52.196 | 121.558 | 1.00 | 141.29 | C |
| ATOM | 15911 | CG | GLU | G | 259 | −16.035 | 53.690 | 121.414 | 1.00 | 139.63 | C |
| ATOM | 15914 | CD | GLU | G | 259 | −17.301 | 54.517 | 121.233 | 1.00 | 138.67 | C |
| ATOM | 15915 | OE1 | GLU | G | 259 | −17.710 | 54.775 | 120.077 | 1.00 | 126.95 | O |
| ATOM | 15916 | OE2 | GLU | G | 259 | −17.903 | 54.913 | 122.252 | 1.00 | 142.20 | O |
| ATOM | 15917 | C | GLU | G | 259 | −17.185 | 50.307 | 120.111 | 1.00 | 137.29 | C |
| ATOM | 15918 | O | GLU | G | 259 | −18.330 | 50.551 | 120.511 | 1.00 | 133.78 | O |
| ATOM | 15919 | N | GLY | G | 260 | −16.856 | 49.161 | 119.511 | 1.00 | 130.95 | N |
| ATOM | 15921 | CA | GLY | G | 260 | −17.362 | 48.804 | 118.195 | 1.00 | 125.70 | C |
| ATOM | 15924 | C | GLY | G | 260 | −16.438 | 47.851 | 117.464 | 1.00 | 121.22 | C |
| ATOM | 15925 | O | GLY | G | 260 | −16.049 | 48.103 | 116.326 | 1.00 | 114.60 | O |
| ATOM | 15926 | N | MET | I | 1 | 31.220 | 62.568 | 44.915 | 1.00 | 77.66 | N |
| ATOM | 15928 | CA | MET | I | 1 | 29.861 | 63.211 | 44.776 | 1.00 | 81.75 | C |
| ATOM | 15930 | CB | MET | I | 1 | 29.852 | 64.307 | 43.705 | 1.00 | 80.20 | C |
| ATOM | 15933 | CG | MET | I | 1 | 28.609 | 65.193 | 43.777 | 1.00 | 91.11 | C |
| ATOM | 15936 | SD | MET | I | 1 | 27.083 | 64.522 | 43.026 | 1.00 | 112.67 | S |
| ATOM | 15937 | CE | MET | I | 1 | 25.801 | 65.712 | 43.697 | 1.00 | 101.89 | C |
| ATOM | 15941 | C | MET | I | 1 | 28.681 | 62.269 | 44.473 | 1.00 | 78.13 | C |
| ATOM | 15942 | O | MET | I | 1 | 28.443 | 61.902 | 43.307 | 1.00 | 71.92 | O |
| ATOM | 15945 | N | PHE | I | 2 | 27.871 | 62.034 | 45.509 | 1.00 | 67.05 | N |
| ATOM | 15947 | CA | PHE | I | 2 | 26.986 | 60.882 | 45.604 | 1.00 | 56.38 | C |
| ATOM | 15949 | CB | PHE | I | 2 | 27.301 | 60.167 | 46.915 | 1.00 | 56.02 | C |
| ATOM | 15952 | CG | PHE | I | 2 | 26.170 | 59.349 | 47.449 | 1.00 | 65.19 | C |
| ATOM | 15953 | CD1 | PHE | I | 2 | 25.491 | 58.471 | 46.618 | 1.00 | 61.21 | C |
| ATOM | 15955 | CE1 | PHE | I | 2 | 24.446 | 57.722 | 47.081 | 1.00 | 58.95 | C |
| ATOM | 15957 | CZ | PHE | I | 2 | 24.052 | 57.843 | 48.390 | 1.00 | 66.04 | C |
| ATOM | 15959 | CE2 | PHE | I | 2 | 24.737 | 58.690 | 49.244 | 1.00 | 68.97 | C |
| ATOM | 15961 | CD2 | PHE | I | 2 | 25.802 | 59.432 | 48.778 | 1.00 | 65.54 | C |
| ATOM | 15963 | C | PHE | I | 2 | 25.539 | 61.359 | 45.611 | 1.00 | 52.36 | C |
| ATOM | 15964 | O | PHE | I | 2 | 25.129 | 62.008 | 46.558 | 1.00 | 57.08 | O |
| ATOM | 15965 | N | GLU | I | 3 | 24.719 | 60.996 | 44.627 | 1.00 | 51.18 | N |
| ATOM | 15967 | CA | GLU | I | 3 | 23.267 | 61.228 | 44.770 | 1.00 | 48.67 | C |
| ATOM | 15969 | CB | GLU | I | 3 | 22.826 | 62.436 | 43.952 | 1.00 | 48.80 | C |
| ATOM | 15972 | CG | GLU | I | 3 | 21.502 | 63.033 | 44.351 | 1.00 | 58.61 | C |
| ATOM | 15975 | CD | GLU | I | 3 | 21.109 | 64.196 | 43.454 | 1.00 | 64.06 | C |
| ATOM | 15976 | OE1 | GLU | I | 3 | 20.375 | 63.978 | 42.484 | 1.00 | 54.80 | O |
| ATOM | 15977 | OE2 | GLU | I | 3 | 21.549 | 65.335 | 43.670 | 1.00 | 78.54 | O |
| ATOM | 15978 | C | GLU | I | 3 | 22.414 | 60.041 | 44.351 | 1.00 | 45.20 | C |
| ATOM | 15979 | O | GLU | I | 3 | 22.405 | 59.662 | 43.185 | 1.00 | 55.83 | O |
| ATOM | 15980 | N | ALA | I | 4 | 21.676 | 59.491 | 45.308 | 1.00 | 44.09 | N |
| ATOM | 15982 | CA | ALA | I | 4 | 20.709 | 58.433 | 45.071 | 1.00 | 41.81 | C |
| ATOM | 15984 | CB | ALA | I | 4 | 21.067 | 57.263 | 45.892 | 1.00 | 42.43 | C |
| ATOM | 15988 | C | ALA | I | 4 | 19.280 | 58.865 | 45.388 | 1.00 | 46.81 | C |
| ATOM | 15989 | O | ALA | I | 4 | 19.026 | 59.542 | 46.372 | 1.00 | 52.37 | O |
| ATOM | 15990 | N | ARG | I | 5 | 18.334 | 58.487 | 44.541 | 1.00 | 49.90 | N |
| ATOM | 15992 | CA | ARG | I | 5 | 16.954 | 58.907 | 44.726 | 1.00 | 45.78 | C |
| ATOM | 15994 | CB | ARG | I | 5 | 16.491 | 59.758 | 43.551 | 1.00 | 53.49 | C |
| ATOM | 15997 | CG | ARG | I | 5 | 15.007 | 60.089 | 43.428 | 1.00 | 44.01 | C |
| ATOM | 16000 | CD | ARG | I | 5 | 14.632 | 60.727 | 42.057 | 1.00 | 44.60 | C |
| ATOM | 16003 | NE | ARG | I | 5 | 13.228 | 60.508 | 41.731 | 1.00 | 63.94 | N |
| ATOM | 16005 | CZ | ARG | I | 5 | 12.264 | 61.364 | 42.065 | 1.00 | 71.74 | C |
| ATOM | 16006 | NH1 | ARG | I | 5 | 12.580 | 62.511 | 42.649 | 1.00 | 59.13 | N |
| ATOM | 16009 | NH2 | ARG | I | 5 | 10.985 | 61.087 | 41.820 | 1.00 | 71.41 | N |
| ATOM | 16012 | C | ARG | I | 5 | 16.180 | 57.626 | 44.735 | 1.00 | 46.20 | C |
| ATOM | 16013 | O | ARG | I | 5 | 16.345 | 56.751 | 43.884 | 1.00 | 43.13 | O |
| ATOM | 16014 | N | LEU | I | 6 | 15.334 | 57.534 | 45.739 | 1.00 | 45.59 | N |
| ATOM | 16016 | CA | LEU | I | 6 | 14.715 | 56.282 | 46.090 | 1.00 | 43.48 | C |
| ATOM | 16018 | CB | LEU | I | 6 | 15.337 | 55.762 | 47.368 | 1.00 | 37.21 | C |
| ATOM | 16021 | CG | LEU | I | 6 | 14.529 | 54.589 | 47.880 | 1.00 | 43.09 | C |
| ATOM | 16023 | CD1 | LEU | I | 6 | 14.833 | 53.401 | 47.009 | 1.00 | 59.15 | C |
| ATOM | 16027 | CD2 | LEU | I | 6 | 14.828 | 54.309 | 49.330 | 1.00 | 46.08 | C |
| ATOM | 16031 | C | LEU | I | 6 | 13.230 | 56.557 | 46.273 | 1.00 | 43.00 | C |
| ATOM | 16032 | O | LEU | I | 6 | 12.813 | 57.251 | 47.158 | 1.00 | 51.65 | O |
| ATOM | 16033 | N | VAL | I | 7 | 12.422 | 56.110 | 45.345 | 1.00 | 47.07 | N |
| ATOM | 16035 | CA | VAL | I | 7 | 11.029 | 56.467 | 45.357 | 1.00 | 43.17 | C |
| ATOM | 16037 | CB | VAL | I | 7 | 10.444 | 55.889 | 44.052 | 1.00 | 47.64 | C |
| ATOM | 16039 | CG1 | VAL | I | 7 | 8.921 | 56.019 | 43.988 | 1.00 | 39.52 | C |
| ATOM | 16043 | CG2 | VAL | I | 7 | 11.165 | 56.468 | 42.865 | 1.00 | 33.69 | C |
| ATOM | 16047 | C | VAL | I | 7 | 10.362 | 55.791 | 46.562 | 1.00 | 44.88 | C |
| ATOM | 16048 | O | VAL | I | 7 | 9.492 | 56.368 | 47.196 | 1.00 | 49.05 | O |
| ATOM | 16049 | N | GLN | I | 8 | 10.719 | 54.535 | 46.811 | 1.00 | 41.93 | N |
| ATOM | 16051 | CA | GLN | I | 8 | 10.098 | 53.746 | 47.863 | 1.00 | 43.67 | C |
| ATOM | 16053 | CB | GLN | I | 8 | 10.136 | 52.241 | 47.562 | 1.00 | 39.36 | C |
| ATOM | 16056 | CG | GLN | I | 8 | 9.136 | 51.483 | 48.428 | 1.00 | 41.08 | C |
| ATOM | 16059 | CD | GLN | I | 8 | 8.963 | 50.013 | 48.107 | 1.00 | 32.18 | C |
| ATOM | 16060 | OE1 | GLN | I | 8 | 8.255 | 49.297 | 48.818 | 1.00 | 41.20 | O |
| ATOM | 16061 | NE2 | GLN | I | 8 | 9.573 | 49.570 | 47.031 | 1.00 | 23.19 | N |
| ATOM | 16064 | C | GLN | I | 8 | 10.860 | 53.983 | 49.158 | 1.00 | 46.15 | C |
| ATOM | 16065 | O | GLN | I | 8 | 11.409 | 53.037 | 49.725 | 1.00 | 46.28 | O |
| ATOM | 16066 | N | GLY | I | 9 | 10.922 | 55.242 | 49.583 | 1.00 | 39.60 | N |
| ATOM | 16068 | CA | GLY | I | 9 | 11.643 | 55.616 | 50.780 | 1.00 | 38.93 | C |
| ATOM | 16071 | C | GLY | I | 9 | 11.248 | 54.984 | 52.111 | 1.00 | 36.11 | C |
| ATOM | 16072 | O | GLY | I | 9 | 12.029 | 55.040 | 53.049 | 1.00 | 34.54 | O |
| ATOM | 16073 | N | SER | I | 10 | 10.053 | 54.414 | 52.226 | 1.00 | 33.52 | N |
| ATOM | 16075 | CA | SER | I | 10 | 9.730 | 53.524 | 53.334 | 1.00 | 29.06 | C |
| ATOM | 16077 | CB | SER | I | 10 | 8.398 | 52.837 | 53.038 | 1.00 | 35.44 | C |
| ATOM | 16080 | OG | SER | I | 10 | 8.060 | 52.920 | 51.658 | 1.00 | 56.04 | O |
| ATOM | 16082 | C | SER | I | 10 | 10.819 | 52.476 | 53.530 | 1.00 | 30.45 | C |
| ATOM | 16083 | O | SER | I | 10 | 11.064 | 51.958 | 54.605 | 1.00 | 33.30 | O |
| ATOM | 16084 | N | ILE | I | 11 | 11.533 | 52.149 | 52.478 | 1.00 | 35.80 | N |
| ATOM | 16086 | CA | ILE | I | 11 | 12.500 | 51.086 | 52.621 | 1.00 | 37.20 | C |
| ATOM | 16088 | CB | ILE | I | 11 | 13.088 | 50.676 | 51.246 | 1.00 | 40.28 | C |
| ATOM | 16090 | CG1 | ILE | I | 11 | 12.158 | 49.703 | 50.517 | 1.00 | 41.59 | C |
| ATOM | 16093 | CD1 | ILE | I | 11 | 12.598 | 49.417 | 49.089 | 1.00 | 45.83 | C |
| ATOM | 16097 | CG2 | ILE | I | 11 | 14.452 | 50.030 | 51.454 | 1.00 | 38.20 | C |
| ATOM | 16101 | C | ILE | I | 11 | 13.591 | 51.605 | 53.527 | 1.00 | 38.73 | C |
| ATOM | 16102 | O | ILE | I | 11 | 14.107 | 50.857 | 54.372 | 1.00 | 33.78 | O |
| ATOM | 16103 | N | LEU | I | 12 | 13.998 | 52.853 | 53.279 | 1.00 | 33.26 | N |
| ATOM | 16105 | CA | LEU | I | 12 | 15.121 | 53.376 | 54.029 | 1.00 | 37.25 | C |
| ATOM | 16107 | CB | LEU | I | 12 | 15.712 | 54.573 | 53.309 | 1.00 | 39.38 | C |
| ATOM | 16110 | CG | LEU | I | 12 | 17.147 | 54.990 | 53.662 | 1.00 | 52.14 | C |
| ATOM | 16112 | CD1 | LEU | I | 12 | 18.234 | 53.941 | 53.445 | 1.00 | 44.91 | C |
| ATOM | 16116 | CD2 | LEU | I | 12 | 17.541 | 56.245 | 52.887 | 1.00 | 56.60 | C |
| ATOM | 16120 | C | LEU | I | 12 | 14.671 | 53.658 | 55.473 | 1.00 | 38.37 | C |
| ATOM | 16121 | O | LEU | I | 12 | 15.364 | 53.412 | 56.454 | 1.00 | 44.66 | O |
| ATOM | 16122 | N | LYS | I | 13 | 13.439 | 54.093 | 55.633 | 1.00 | 42.34 | N |
| ATOM | 16124 | CA | LYS | I | 13 | 12.801 | 54.073 | 56.939 | 1.00 | 36.91 | C |
| ATOM | 16126 | CB | LYS | I | 13 | 11.342 | 54.493 | 56.753 | 1.00 | 32.80 | C |
| ATOM | 16129 | CG | LYS | I | 13 | 11.246 | 55.997 | 56.718 | 1.00 | 37.11 | C |
| ATOM | 16132 | CD | LYS | I | 13 | 9.971 | 56.491 | 56.088 | 1.00 | 45.44 | C |
| ATOM | 16135 | CE | LYS | I | 13 | 8.815 | 56.700 | 57.064 | 1.00 | 40.96 | C |
| ATOM | 16138 | NZ | LYS | I | 13 | 9.233 | 57.425 | 58.270 | 1.00 | 42.18 | N |
| ATOM | 16142 | C | LYS | I | 13 | 12.926 | 52.708 | 57.596 | 1.00 | 38.82 | C |
| ATOM | 16143 | O | LYS | I | 13 | 13.449 | 52.573 | 58.706 | 1.00 | 41.89 | O |
| ATOM | 16144 | N | LYS | I | 14 | 12.433 | 51.680 | 56.922 | 1.00 | 39.47 | N |
| ATOM | 16146 | CA | LYS | I | 14 | 12.388 | 50.364 | 57.548 | 1.00 | 40.69 | C |
| ATOM | 16148 | CB | LYS | I | 14 | 11.548 | 49.381 | 56.720 | 1.00 | 38.95 | C |
| ATOM | 16151 | CG | LYS | I | 14 | 10.046 | 49.659 | 56.800 | 1.00 | 47.86 | C |
| ATOM | 16154 | CD | LYS | I | 14 | 9.134 | 48.623 | 56.139 | 1.00 | 59.92 | C |
| ATOM | 16157 | CE | LYS | I | 14 | 7.922 | 49.267 | 55.466 | 1.00 | 56.03 | C |
| ATOM | 16160 | NZ | LYS | I | 14 | 7.557 | 48.577 | 54.198 | 1.00 | 53.43 | N |
| ATOM | 16164 | C | LYS | I | 14 | 13.812 | 49.830 | 57.844 | 1.00 | 40.66 | C |
| ATOM | 16165 | O | LYS | I | 14 | 14.043 | 49.170 | 58.860 | 1.00 | 41.79 | O |
| ATOM | 16166 | N | VAL | I | 15 | 14.788 | 50.185 | 57.016 | 1.00 | 38.16 | N |
| ATOM | 16168 | CA | VAL | I | 15 | 16.154 | 49.700 | 57.225 | 1.00 | 39.83 | C |
| ATOM | 16170 | CB | VAL | I | 15 | 17.071 | 50.043 | 56.017 | 1.00 | 33.59 | C |
| ATOM | 16172 | CG1 | VAL | I | 15 | 18.495 | 50.196 | 56.476 | 1.00 | 37.59 | C |
| ATOM | 16176 | CG2 | VAL | I | 15 | 16.975 | 49.007 | 54.955 | 1.00 | 42.96 | C |
| ATOM | 16180 | C | VAL | I | 15 | 16.731 | 50.301 | 58.520 | 1.00 | 37.30 | C |
| ATOM | 16181 | O | VAL | I | 15 | 17.363 | 49.624 | 59.345 | 1.00 | 32.70 | O |
| ATOM | 16182 | N | LEU | I | 16 | 16.531 | 51.596 | 58.709 | 1.00 | 34.91 | N |
| ATOM | 16184 | CA | LEU | I | 16 | 17.067 | 52.190 | 59.926 | 1.00 | 37.86 | C |
| ATOM | 16186 | CB | LEU | I | 16 | 17.047 | 53.705 | 59.859 | 1.00 | 29.26 | C |
| ATOM | 16189 | CG | LEU | I | 16 | 18.488 | 53.962 | 59.502 | 1.00 | 41.44 | C |
| ATOM | 16191 | CD1 | LEU | I | 16 | 18.619 | 54.297 | 58.019 | 1.00 | 45.07 | C |
| ATOM | 16195 | CD2 | LEU | I | 16 | 19.052 | 54.977 | 60.468 | 1.00 | 38.13 | C |
| ATOM | 16199 | C | LEU | I | 16 | 16.385 | 51.711 | 61.192 | 1.00 | 33.61 | C |
| ATOM | 16200 | O | LEU | I | 16 | 17.021 | 51.424 | 62.181 | 1.00 | 33.08 | O |
| ATOM | 16201 | N | GLU | I | 17 | 15.078 | 51.552 | 61.153 | 1.00 | 38.92 | N |
| ATOM | 16203 | CA | GLU | I | 17 | 14.453 | 50.871 | 62.261 | 1.00 | 42.53 | C |
| ATOM | 16205 | CB | GLU | I | 17 | 12.954 | 50.785 | 61.994 | 1.00 | 39.84 | C |
| ATOM | 16208 | CG | GLU | I | 17 | 12.282 | 52.135 | 62.132 | 1.00 | 44.03 | C |
| ATOM | 16211 | CD | GLU | I | 17 | 12.309 | 52.613 | 63.569 | 1.00 | 59.02 | C |
| ATOM | 16212 | OE1 | GLU | I | 17 | 11.795 | 51.871 | 64.452 | 1.00 | 49.57 | O |
| ATOM | 16213 | OE2 | GLU | I | 17 | 12.904 | 53.694 | 63.803 | 1.00 | 57.79 | O |
| ATOM | 16214 | C | GLU | I | 17 | 15.083 | 49.511 | 62.569 | 1.00 | 43.90 | C |
| ATOM | 16215 | O | GLU | I | 17 | 15.064 | 49.071 | 63.709 | 1.00 | 59.25 | O |
| ATOM | 16216 | N | ALA | I | 18 | 15.662 | 48.852 | 61.571 | 1.00 | 52.64 | N |
| ATOM | 16218 | CA | ALA | I | 18 | 16.236 | 47.516 | 61.755 | 1.00 | 51.79 | C |
| ATOM | 16220 | CB | ALA | I | 18 | 16.213 | 46.720 | 60.443 | 1.00 | 51.38 | C |
| ATOM | 16224 | C | ALA | I | 18 | 17.664 | 47.561 | 62.284 | 1.00 | 45.32 | C |
| ATOM | 16225 | O | ALA | I | 18 | 18.295 | 46.519 | 62.501 | 1.00 | 38.19 | O |
| ATOM | 16226 | N | LEU | I | 19 | 18.204 | 48.761 | 62.401 | 1.00 | 37.58 | N |
| ATOM | 16228 | CA | LEU | I | 19 | 19.616 | 48.841 | 62.665 | 1.00 | 39.82 | C |
| ATOM | 16230 | CB | LEU | I | 19 | 20.326 | 49.553 | 61.537 | 1.00 | 41.69 | C |
| ATOM | 16233 | CG | LEU | I | 19 | 20.574 | 48.735 | 60.284 | 1.00 | 47.50 | C |
| ATOM | 16235 | CD1 | LEU | I | 19 | 21.306 | 49.656 | 59.314 | 1.00 | 33.68 | C |
| ATOM | 16239 | CD2 | LEU | I | 19 | 21.381 | 47.501 | 60.670 | 1.00 | 40.01 | C |
| ATOM | 16243 | C | LEU | I | 19 | 19.835 | 49.579 | 63.959 | 1.00 | 42.42 | C |
| ATOM | 16244 | O | LEU | I | 19 | 20.753 | 49.235 | 64.684 | 1.00 | 45.97 | O |
| ATOM | 16245 | N | LYS | I | 20 | 19.002 | 50.577 | 64.258 | 1.00 | 46.28 | N |
| ATOM | 16247 | CA | LYS | I | 20 | 19.322 | 51.595 | 65.270 | 1.00 | 42.76 | C |
| ATOM | 16249 | CB | LYS | I | 20 | 18.383 | 52.781 | 65.178 | 1.00 | 39.62 | C |
| ATOM | 16252 | CG | LYS | I | 20 | 16.932 | 52.460 | 65.529 | 1.00 | 48.10 | C |
| ATOM | 16255 | CD | LYS | I | 20 | 16.145 | 53.737 | 65.775 | 1.00 | 57.02 | C |
| ATOM | 16258 | CE | LYS | I | 20 | 14.790 | 53.460 | 66.380 | 1.00 | 62.97 | C |
| ATOM | 16261 | NZ | LYS | I | 20 | 14.951 | 52.408 | 67.390 | 1.00 | 44.30 | N |
| ATOM | 16265 | C | LYS | I | 20 | 19.365 | 51.068 | 66.709 | 1.00 | 38.34 | C |
| ATOM | 16266 | O | LYS | I | 20 | 20.289 | 51.360 | 67.450 | 1.00 | 47.67 | O |
| ATOM | 16267 | N | ASP | I | 21 | 18.479 | 50.157 | 67.053 | 1.00 | 39.55 | N |
| ATOM | 16269 | CA | ASP | I | 21 | 18.579 | 49.475 | 68.337 | 1.00 | 43.56 | C |
| ATOM | 16271 | CB | ASP | I | 21 | 17.307 | 48.676 | 68.542 | 1.00 | 45.49 | C |
| ATOM | 16274 | CG | ASP | I | 21 | 16.117 | 49.598 | 68.688 | 1.00 | 56.52 | C |
| ATOM | 16275 | OD1 | ASP | I | 21 | 16.390 | 50.810 | 68.908 | 1.00 | 55.23 | O |
| ATOM | 16276 | OD2 | ASP | I | 21 | 14.923 | 49.226 | 68.607 | 1.00 | 47.58 | O |
| ATOM | 16277 | C | ASP | I | 21 | 19.774 | 48.582 | 68.555 | 1.00 | 38.56 | C |
| ATOM | 16278 | O | ASP | I | 21 | 20.226 | 48.384 | 69.669 | 1.00 | 44.82 | O |
| ATOM | 16279 | N | LEU | I | 22 | 20.294 | 48.022 | 67.484 | 1.00 | 45.49 | N |
| ATOM | 16281 | CA | LEU | I | 22 | 21.404 | 47.093 | 67.616 | 1.00 | 45.36 | C |
| ATOM | 16283 | CB | LEU | I | 22 | 21.381 | 46.123 | 66.452 | 1.00 | 43.08 | C |
| ATOM | 16286 | CG | LEU | I | 22 | 22.461 | 45.060 | 66.560 | 1.00 | 36.01 | C |
| ATOM | 16288 | CD1 | LEU | I | 22 | 22.448 | 44.338 | 67.885 | 1.00 | 35.07 | C |
| ATOM | 16292 | CD2 | LEU | I | 22 | 22.207 | 44.101 | 65.450 | 1.00 | 28.65 | C |
| ATOM | 16296 | C | LEU | I | 22 | 22.756 | 47.799 | 67.628 | 1.00 | 45.26 | C |
| ATOM | 16297 | O | LEU | I | 22 | 23.629 | 47.461 | 68.418 | 1.00 | 50.39 | O |
| ATOM | 16298 | N | ILE | I | 23 | 22.937 | 48.741 | 66.711 | 1.00 | 44.68 | N |
| ATOM | 16300 | CA | ILE | I | 23 | 24.180 | 49.481 | 66.617 | 1.00 | 47.37 | C |
| ATOM | 16302 | CB | ILE | I | 23 | 25.002 | 48.967 | 65.445 | 1.00 | 47.97 | C |
| ATOM | 16304 | CG1 | ILE | I | 23 | 24.258 | 49.098 | 64.146 | 1.00 | 51.28 | C |
| ATOM | 16307 | CD1 | ILE | I | 23 | 25.216 | 49.340 | 63.040 | 1.00 | 67.94 | C |
| ATOM | 16311 | CG2 | ILE | I | 23 | 25.209 | 47.479 | 65.569 | 1.00 | 50.29 | C |
| ATOM | 16315 | C | ILE | I | 23 | 23.946 | 50.964 | 66.492 | 1.00 | 45.84 | C |
| ATOM | 16316 | O | ILE | I | 23 | 22.980 | 51.399 | 65.884 | 1.00 | 44.44 | O |
| ATOM | 16317 | N | ASN | I | 24 | 24.768 | 51.746 | 67.169 | 1.00 | 48.66 | N |
| ATOM | 16319 | CA | ASN | I | 24 | 24.420 | 53.151 | 67.362 | 1.00 | 54.25 | C |
| ATOM | 16321 | CB | ASN | I | 24 | 24.723 | 53.620 | 68.794 | 1.00 | 56.19 | C |
| ATOM | 16324 | CG | ASN | I | 24 | 23.598 | 54.468 | 69.372 | 1.00 | 72.22 | C |
| ATOM | 16325 | OD1 | ASN | I | 24 | 22.595 | 53.926 | 69.878 | 1.00 | 76.08 | O |
| ATOM | 16326 | ND2 | ASN | I | 24 | 23.730 | 55.806 | 69.249 | 1.00 | 41.59 | N |
| ATOM | 16329 | C | ASN | I | 24 | 25.118 | 54.071 | 66.363 | 1.00 | 51.77 | C |
| ATOM | 16330 | O | ASN | I | 24 | 24.700 | 55.211 | 66.160 | 1.00 | 54.42 | O |
| ATOM | 16331 | N | GLU | I | 25 | 26.236 | 53.616 | 65.811 | 1.00 | 53.85 | N |
| ATOM | 16333 | CA | GLU | I | 25 | 26.989 | 54.394 | 64.842 | 1.00 | 52.46 | C |
| ATOM | 16335 | CB | GLU | I | 25 | 28.033 | 55.293 | 65.502 | 1.00 | 51.23 | C |
| ATOM | 16338 | CG | GLU | I | 25 | 28.874 | 54.580 | 66.540 | 1.00 | 70.32 | C |
| ATOM | 16341 | CD | GLU | I | 25 | 29.462 | 55.527 | 67.570 | 1.00 | 88.46 | C |
| ATOM | 16342 | OE1 | GLU | I | 25 | 30.696 | 55.449 | 67.730 | 1.00 | 67.14 | O |
| ATOM | 16343 | OE2 | GLU | I | 25 | 28.717 | 56.306 | 68.226 | 1.00 | 91.11 | O |
| ATOM | 16344 | C | GLU | I | 25 | 27.660 | 53.389 | 63.959 | 1.00 | 45.02 | C |
| ATOM | 16345 | O | GLU | I | 25 | 28.215 | 52.411 | 64.424 | 1.00 | 53.70 | O |
| ATOM | 16346 | N | ALA | I | 26 | 27.565 | 53.598 | 62.664 | 1.00 | 41.99 | N |
| ATOM | 16348 | CA | ALA | I | 26 | 28.264 | 52.718 | 61.759 | 1.00 | 43.01 | C |
| ATOM | 16350 | CB | ALA | I | 26 | 27.437 | 51.514 | 61.463 | 1.00 | 46.87 | C |
| ATOM | 16354 | C | ALA | I | 26 | 28.610 | 53.470 | 60.491 | 1.00 | 41.52 | C |
| ATOM | 16355 | O | ALA | I | 26 | 28.128 | 54.572 | 60.259 | 1.00 | 48.10 | O |
| ATOM | 16356 | N | CYS | I | 27 | 29.469 | 52.870 | 59.689 | 1.00 | 37.66 | N |
| ATOM | 16358 | CA | CYS | I | 27 | 29.963 | 53.509 | 58.494 | 1.00 | 42.84 | C |
| ATOM | 16360 | CB | CYS | I | 27 | 31.467 | 53.227 | 58.363 | 1.00 | 46.33 | C |
| ATOM | 16363 | SG | CYS | I | 27 | 32.155 | 53.824 | 56.822 | 1.00 | 56.32 | S |
| ATOM | 16364 | C | CYS | I | 27 | 29.200 | 52.984 | 57.287 | 1.00 | 29.77 | C |
| ATOM | 16365 | O | CYS | I | 27 | 29.211 | 51.808 | 57.030 | 1.00 | 51.64 | O |
| ATOM | 16366 | N | TRP | I | 28 | 28.548 | 53.862 | 56.547 | 1.00 | 41.94 | N |
| ATOM | 16368 | CA | TRP | I | 28 | 27.919 | 53.559 | 55.258 | 1.00 | 43.81 | C |
| ATOM | 16370 | CB | TRP | I | 28 | 26.760 | 54.525 | 54.963 | 1.00 | 42.73 | C |
| ATOM | 16373 | CG | TRP | I | 28 | 25.673 | 54.416 | 55.995 | 1.00 | 44.11 | C |
| ATOM | 16374 | CD1 | TRP | I | 28 | 25.771 | 54.699 | 57.327 | 1.00 | 49.13 | C |
| ATOM | 16376 | NE1 | TRP | I | 28 | 24.577 | 54.446 | 57.955 | 1.00 | 39.47 | N |
| ATOM | 16378 | CE2 | TRP | I | 28 | 23.676 | 54.018 | 57.023 | 1.00 | 54.56 | C |
| ATOM | 16379 | CD2 | TRP | I | 28 | 24.336 | 53.975 | 55.785 | 1.00 | 50.04 | C |
| ATOM | 16380 | CE3 | TRP | I | 28 | 23.621 | 53.558 | 54.670 | 1.00 | 39.53 | C |
| ATOM | 16382 | CZ3 | TRP | I | 28 | 22.338 | 53.208 | 54.817 | 1.00 | 49.30 | C |
| ATOM | 16384 | CH2 | TRP | I | 28 | 21.719 | 53.222 | 56.052 | 1.00 | 67.51 | C |
| ATOM | 16386 | CZ2 | TRP | I | 28 | 22.364 | 53.635 | 57.171 | 1.00 | 68.27 | C |
| ATOM | 16388 | C | TRP | I | 28 | 28.954 | 53.775 | 54.202 | 1.00 | 42.28 | C |
| ATOM | 16389 | O | TRP | I | 28 | 29.434 | 54.893 | 54.030 | 1.00 | 45.35 | O |
| ATOM | 16390 | N | ASP | I | 29 | 29.316 | 52.679 | 53.550 | 1.00 | 49.84 | N |
| ATOM | 16392 | CA | ASP | I | 29 | 30.312 | 52.674 | 52.485 | 1.00 | 51.14 | C |
| ATOM | 16394 | CB | ASP | I | 29 | 30.997 | 51.304 | 52.429 | 1.00 | 50.59 | C |
| ATOM | 16397 | CG | ASP | I | 29 | 32.080 | 51.150 | 53.495 | 1.00 | 64.57 | C |
| ATOM | 16398 | OD1 | ASP | I | 29 | 32.890 | 52.087 | 53.625 | 1.00 | 76.15 | O |
| ATOM | 16399 | OD2 | ASP | I | 29 | 32.203 | 50.165 | 54.259 | 1.00 | 68.68 | O |
| ATOM | 16400 | C | ASP | I | 29 | 29.551 | 52.926 | 51.214 | 1.00 | 43.80 | C |
| ATOM | 16401 | O | ASP | I | 29 | 28.709 | 52.132 | 50.840 | 1.00 | 55.30 | O |
| ATOM | 16402 | N | ILE | I | 30 | 29.786 | 54.051 | 50.567 | 1.00 | 50.68 | N |
| ATOM | 16404 | CA | ILE | I | 30 | 29.172 | 54.247 | 49.249 | 1.00 | 57.45 | C |
| ATOM | 16406 | CB | ILE | I | 30 | 28.528 | 55.648 | 49.167 | 1.00 | 55.74 | C |
| ATOM | 16408 | CG1 | ILE | I | 30 | 27.303 | 55.682 | 50.074 | 1.00 | 53.23 | C |
| ATOM | 16411 | CD1 | ILE | I | 30 | 27.651 | 55.922 | 51.496 | 1.00 | 60.70 | C |
| ATOM | 16415 | CG2 | ILE | I | 30 | 28.060 | 55.921 | 47.747 | 1.00 | 46.12 | C |
| ATOM | 16419 | C | ILE | I | 30 | 30.083 | 53.992 | 48.035 | 1.00 | 54.89 | C |
| ATOM | 16420 | O | ILE | I | 30 | 31.265 | 54.347 | 48.060 | 1.00 | 56.46 | O |
| ATOM | 16421 | N | SER | I | 31 | 29.536 | 53.372 | 46.990 | 1.00 | 56.10 | N |
| ATOM | 16423 | CA | SER | I | 31 | 30.268 | 53.173 | 45.726 | 1.00 | 56.31 | C |
| ATOM | 16425 | CB | SER | I | 31 | 31.076 | 51.872 | 45.785 | 1.00 | 55.05 | C |
| ATOM | 16428 | OG | SER | I | 31 | 30.195 | 50.754 | 45.750 | 1.00 | 45.69 | O |
| ATOM | 16430 | C | SER | I | 31 | 29.341 | 53.113 | 44.495 | 1.00 | 55.60 | C |
| ATOM | 16431 | O | SER | I | 31 | 28.123 | 53.293 | 44.613 | 1.00 | 48.77 | O |
| ATOM | 16432 | N | SER | I | 32 | 29.929 | 52.820 | 43.330 | 1.00 | 54.86 | N |
| ATOM | 16434 | CA | SER | I | 32 | 29.180 | 52.684 | 42.083 | 1.00 | 49.80 | C |
| ATOM | 16436 | CB | SER | I | 32 | 30.091 | 52.312 | 40.933 | 1.00 | 42.76 | C |
| ATOM | 16439 | OG | SER | I | 32 | 30.911 | 53.402 | 40.644 | 1.00 | 63.63 | O |
| ATOM | 16441 | C | SER | I | 32 | 28.178 | 51.571 | 42.211 | 1.00 | 49.90 | C |
| ATOM | 16442 | O | SER | I | 32 | 27.098 | 51.620 | 41.627 | 1.00 | 51.99 | O |
| ATOM | 16443 | N | SER | I | 33 | 28.588 | 50.504 | 42.877 | 1.00 | 46.24 | N |
| ATOM | 16445 | CA | SER | I | 33 | 27.759 | 49.324 | 42.877 | 1.00 | 46.19 | C |
| ATOM | 16447 | CB | SER | I | 33 | 28.622 | 48.146 | 43.298 | 1.00 | 48.76 | C |
| ATOM | 16450 | OG | SER | I | 33 | 29.337 | 48.451 | 44.477 | 1.00 | 57.37 | O |
| ATOM | 16452 | C | SER | I | 33 | 26.600 | 49.536 | 43.858 | 1.00 | 50.46 | C |
| ATOM | 16453 | O | SER | I | 33 | 25.567 | 48.864 | 43.784 | 1.00 | 48.14 | O |
| ATOM | 16454 | N | GLY | I | 34 | 26.796 | 50.479 | 44.777 | 1.00 | 51.55 | N |
| ATOM | 16456 | CA | GLY | I | 34 | 25.725 | 51.005 | 45.607 | 1.00 | 54.00 | C |
| ATOM | 16459 | C | GLY | I | 34 | 26.148 | 51.119 | 47.062 | 1.00 | 52.98 | C |
| ATOM | 16460 | O | GLY | I | 34 | 27.302 | 51.482 | 47.350 | 1.00 | 60.56 | O |
| ATOM | 16461 | N | VAL | I | 35 | 25.231 | 50.804 | 47.978 | 1.00 | 46.69 | N |
| ATOM | 16463 | CA | VAL | I | 35 | 25.430 | 51.150 | 49.391 | 1.00 | 48.87 | C |
| ATOM | 16465 | CB | VAL | I | 35 | 24.195 | 51.793 | 50.030 | 1.00 | 47.50 | C |
| ATOM | 16467 | CG1 | VAL | I | 35 | 24.481 | 51.989 | 51.491 | 1.00 | 34.67 | C |
| ATOM | 16471 | CG2 | VAL | I | 35 | 23.851 | 53.101 | 49.379 | 1.00 | 47.30 | C |
| ATOM | 16475 | C | VAL | I | 35 | 25.681 | 49.941 | 50.272 | 1.00 | 46.18 | C |
| ATOM | 16476 | O | VAL | I | 35 | 24.804 | 49.090 | 50.370 | 1.00 | 53.01 | O |
| ATOM | 16477 | N | ASN | I | 36 | 26.811 | 49.890 | 50.970 | 1.00 | 45.22 | N |
| ATOM | 16479 | CA | ASN | I | 36 | 27.052 | 48.792 | 51.908 | 1.00 | 50.82 | C |
| ATOM | 16481 | CB | ASN | I | 36 | 28.154 | 47.868 | 51.416 | 1.00 | 49.58 | C |
| ATOM | 16484 | CG | ASN | I | 36 | 27.805 | 47.181 | 50.095 | 1.00 | 74.32 | C |
| ATOM | 16485 | OD1 | ASN | I | 36 | 27.902 | 45.958 | 49.993 | 1.00 | 92.85 | O |
| ATOM | 16486 | ND2 | ASN | I | 36 | 27.463 | 47.960 | 49.064 | 1.00 | 92.03 | N |
| ATOM | 16489 | C | ASN | I | 36 | 27.368 | 49.234 | 53.341 | 1.00 | 50.35 | C |
| ATOM | 16490 | O | ASN | I | 36 | 27.961 | 50.297 | 53.565 | 1.00 | 45.75 | O |
| ATOM | 16491 | N | LEU | I | 37 | 26.946 | 48.407 | 54.300 | 1.00 | 42.50 | N |
| ATOM | 16493 | CA | LEU | I | 37 | 27.128 | 48.689 | 55.727 | 1.00 | 38.72 | C |
| ATOM | 16495 | CB | LEU | I | 37 | 25.844 | 49.276 | 56.309 | 1.00 | 39.11 | C |
| ATOM | 16498 | CG | LEU | I | 37 | 26.018 | 49.787 | 57.732 | 1.00 | 48.67 | C |
| ATOM | 16500 | CD1 | LEU | I | 37 | 25.219 | 51.058 | 57.986 | 1.00 | 55.48 | C |
| ATOM | 16504 | CD2 | LEU | I | 37 | 25.735 | 48.682 | 58.747 | 1.00 | 51.60 | C |
| ATOM | 16508 | C | LEU | I | 37 | 27.509 | 47.434 | 56.537 | 1.00 | 40.49 | C |
| ATOM | 16509 | O | LEU | I | 37 | 26.812 | 46.423 | 56.486 | 1.00 | 44.54 | O |
| ATOM | 16510 | N | GLN | I | 38 | 28.615 | 47.480 | 57.281 | 1.00 | 33.08 | N |
| ATOM | 16512 | CA | GLN | I | 38 | 28.969 | 46.346 | 58.117 | 1.00 | 30.43 | C |
| ATOM | 16514 | CB | GLN | I | 38 | 30.123 | 45.617 | 57.453 | 1.00 | 31.90 | C |
| ATOM | 16517 | CG | GLN | I | 38 | 31.074 | 44.841 | 58.355 | 1.00 | 51.51 | C |
| ATOM | 16520 | CD | GLN | I | 38 | 31.328 | 43.459 | 57.799 | 1.00 | 63.22 | C |
| ATOM | 16521 | OE1 | GLN | I | 38 | 32.321 | 42.842 | 58.129 | 1.00 | 52.52 | O |
| ATOM | 16522 | NE2 | GLN | I | 38 | 30.404 | 42.951 | 56.982 | 1.00 | 88.58 | N |
| ATOM | 16525 | C | GLN | I | 38 | 29.330 | 46.845 | 59.510 | 1.00 | 29.18 | C |
| ATOM | 16526 | O | GLN | I | 38 | 30.063 | 47.778 | 59.670 | 1.00 | 39.17 | O |
| ATOM | 16527 | N | SER | I | 39 | 28.780 | 46.280 | 60.556 | 1.00 | 34.73 | N |
| ATOM | 16529 | CA | SER | I | 39 | 29.222 | 46.698 | 61.865 | 1.00 | 38.39 | C |
| ATOM | 16531 | CB | SER | I | 39 | 28.449 | 47.925 | 62.294 | 1.00 | 46.83 | C |
| ATOM | 16534 | OG | SER | I | 39 | 28.920 | 48.333 | 63.554 | 1.00 | 53.68 | O |
| ATOM | 16536 | C | SER | I | 39 | 28.901 | 45.621 | 62.840 | 1.00 | 39.33 | C |
| ATOM | 16537 | O | SER | I | 39 | 27.894 | 44.966 | 62.707 | 1.00 | 46.70 | O |
| ATOM | 16538 | N | MET | I | 40 | 29.718 | 45.466 | 63.860 | 1.00 | 50.77 | N |
| ATOM | 16540 | CA | MET | I | 40 | 29.388 | 44.573 | 64.969 | 1.00 | 49.80 | C |
| ATOM | 16542 | CB | MET | I | 40 | 30.676 | 44.048 | 65.587 | 1.00 | 48.87 | C |
| ATOM | 16545 | CG | MET | I | 40 | 31.514 | 43.280 | 64.606 | 1.00 | 52.31 | C |
| ATOM | 16548 | SD | MET | I | 40 | 32.780 | 42.272 | 65.424 | 1.00 | 60.58 | S |
| ATOM | 16549 | CE | MET | I | 40 | 33.701 | 43.458 | 66.220 | 1.00 | 30.57 | C |
| ATOM | 16553 | C | MET | I | 40 | 28.671 | 45.381 | 66.029 | 1.00 | 41.02 | C |
| ATOM | 16554 | O | MET | I | 40 | 28.672 | 46.602 | 65.992 | 1.00 | 43.91 | O |
| ATOM | 16555 | N | ASP | I | 41 | 28.105 | 44.710 | 67.010 | 1.00 | 27.17 | N |
| ATOM | 16557 | CA | ASP | I | 41 | 27.539 | 45.468 | 68.094 | 1.00 | 40.78 | C |
| ATOM | 16559 | CB | ASP | I | 41 | 26.328 | 44.785 | 68.691 | 1.00 | 35.02 | C |
| ATOM | 16562 | CG | ASP | I | 41 | 26.678 | 43.525 | 69.410 | 1.00 | 32.55 | C |
| ATOM | 16563 | OD1 | ASP | I | 41 | 26.866 | 42.502 | 68.736 | 1.00 | 56.30 | O |
| ATOM | 16564 | OD2 | ASP | I | 41 | 26.709 | 43.430 | 70.652 | 1.00 | 57.80 | O |
| ATOM | 16565 | C | ASP | I | 41 | 28.641 | 45.633 | 69.098 | 1.00 | 48.50 | C |
| ATOM | 16566 | O | ASP | I | 41 | 29.694 | 45.019 | 68.945 | 1.00 | 51.84 | O |
| ATOM | 16567 | N | SER | I | 42 | 28.435 | 46.535 | 70.053 | 1.00 | 55.58 | N |
| ATOM | 16569 | CA | SER | I | 42 | 29.453 | 46.820 | 71.059 | 1.00 | 56.75 | C |
| ATOM | 16571 | CB | SER | I | 42 | 28.871 | 47.691 | 72.168 | 1.00 | 52.40 | C |
| ATOM | 16574 | OG | SER | I | 42 | 28.061 | 46.884 | 73.019 | 1.00 | 70.08 | O |
| ATOM | 16576 | C | SER | I | 42 | 30.049 | 45.535 | 71.647 | 1.00 | 53.79 | C |
| ATOM | 16577 | O | SER | I | 42 | 31.227 | 45.488 | 72.039 | 1.00 | 56.27 | O |
| ATOM | 16578 | N | SER | I | 43 | 29.245 | 44.487 | 71.733 | 1.00 | 45.23 | N |
| ATOM | 16580 | CA | SER | I | 43 | 29.746 | 43.286 | 72.380 | 1.00 | 46.54 | C |
| ATOM | 16582 | CB | SER | I | 43 | 28.635 | 42.659 | 73.209 | 1.00 | 48.06 | C |
| ATOM | 16585 | OG | SER | I | 43 | 27.829 | 41.801 | 72.427 | 1.00 | 32.04 | O |
| ATOM | 16587 | C | SER | I | 43 | 30.378 | 42.255 | 71.440 | 1.00 | 50.97 | C |
| ATOM | 16588 | O | SER | I | 43 | 30.767 | 41.176 | 71.883 | 1.00 | 53.88 | O |
| ATOM | 16589 | N | HIS | I | 44 | 30.478 | 42.580 | 70.153 | 1.00 | 52.65 | N |
| ATOM | 16591 | CA | HIS | I | 44 | 31.165 | 41.735 | 69.174 | 1.00 | 48.38 | C |
| ATOM | 16593 | CB | HIS | I | 44 | 32.655 | 41.702 | 69.476 | 1.00 | 46.41 | C |
| ATOM | 16596 | CG | HIS | I | 44 | 33.328 | 43.035 | 69.357 | 1.00 | 42.74 | C |
| ATOM | 16597 | ND1 | HIS | I | 44 | 34.697 | 43.178 | 69.423 | 1.00 | 57.86 | N |
| ATOM | 16599 | CE1 | HIS | I | 44 | 35.014 | 44.449 | 69.257 | 1.00 | 59.53 | C |
| ATOM | 16601 | NE2 | HIS | I | 44 | 33.908 | 45.133 | 69.042 | 1.00 | 42.26 | N |
| ATOM | 16603 | CD2 | HIS | I | 44 | 32.838 | 44.272 | 69.115 | 1.00 | 53.66 | C |
| ATOM | 16605 | C | HIS | I | 44 | 30.609 | 40.305 | 69.167 | 1.00 | 47.83 | C |
| ATOM | 16606 | O | HIS | I | 44 | 31.268 | 39.341 | 68.722 | 1.00 | 29.88 | O |
| ATOM | 16607 | N | VAL | I | 45 | 29.372 | 40.179 | 69.642 | 1.00 | 40.07 | N |
| ATOM | 16609 | CA | VAL | I | 45 | 28.728 | 38.884 | 69.632 | 1.00 | 38.12 | C |
| ATOM | 16611 | CB | VAL | I | 45 | 27.854 | 38.731 | 70.879 | 1.00 | 41.49 | C |
| ATOM | 16613 | CG1 | VAL | I | 45 | 26.963 | 37.510 | 70.827 | 1.00 | 41.35 | C |
| ATOM | 16617 | CG2 | VAL | I | 45 | 28.728 | 38.631 | 72.101 | 1.00 | 51.72 | C |
| ATOM | 16621 | C | VAL | I | 45 | 27.937 | 38.654 | 68.358 | 1.00 | 40.01 | C |
| ATOM | 16622 | O | VAL | I | 45 | 27.776 | 37.501 | 67.939 | 1.00 | 37.68 | O |
| ATOM | 16623 | N | SER | I | 46 | 27.427 | 39.750 | 67.787 | 1.00 | 38.50 | N |
| ATOM | 16625 | CA | SER | I | 46 | 26.622 | 39.752 | 66.577 | 1.00 | 32.91 | C |
| ATOM | 16627 | CB | SER | I | 46 | 25.210 | 40.172 | 66.897 | 1.00 | 45.83 | C |
| ATOM | 16630 | OG | SER | I | 46 | 25.142 | 41.596 | 66.907 | 1.00 | 52.09 | O |
| ATOM | 16632 | C | SER | I | 46 | 27.143 | 40.789 | 65.584 | 1.00 | 38.34 | C |
| ATOM | 16633 | O | SER | I | 46 | 27.770 | 41.769 | 65.950 | 1.00 | 41.41 | O |
| ATOM | 16634 | N | LEU | I | 47 | 26.808 | 40.605 | 64.313 | 1.00 | 38.78 | N |
| ATOM | 16636 | CA | LEU | I | 47 | 27.282 | 41.492 | 63.271 | 1.00 | 37.84 | C |
| ATOM | 16638 | CB | LEU | I | 47 | 28.445 | 40.848 | 62.507 | 1.00 | 42.35 | C |
| ATOM | 16641 | CG | LEU | I | 47 | 29.004 | 41.641 | 61.314 | 1.00 | 47.10 | C |
| ATOM | 16643 | CD1 | LEU | I | 47 | 30.488 | 41.422 | 61.328 | 1.00 | 63.69 | C |
| ATOM | 16647 | CD2 | LEU | I | 47 | 28.430 | 41.247 | 59.956 | 1.00 | 53.88 | C |
| ATOM | 16651 | C | LEU | I | 47 | 26.145 | 41.686 | 62.309 | 1.00 | 37.20 | C |
| ATOM | 16652 | O | LEU | I | 47 | 25.454 | 40.732 | 61.966 | 1.00 | 49.88 | O |
| ATOM | 16653 | N | VAL | I | 48 | 26.029 | 42.879 | 61.758 | 1.00 | 40.50 | N |
| ATOM | 16655 | CA | VAL | I | 48 | 25.017 | 43.124 | 60.746 | 1.00 | 43.51 | C |
| ATOM | 16657 | CB | VAL | I | 48 | 24.005 | 44.145 | 61.287 | 1.00 | 49.67 | C |
| ATOM | 16659 | CG1 | VAL | I | 48 | 24.688 | 45.485 | 61.651 | 1.00 | 48.96 | C |
| ATOM | 16663 | CG2 | VAL | I | 48 | 22.872 | 44.325 | 60.305 | 1.00 | 59.17 | C |
| ATOM | 16667 | C | VAL | I | 48 | 25.617 | 43.579 | 59.422 | 1.00 | 35.91 | C |
| ATOM | 16668 | O | VAL | I | 48 | 26.547 | 44.383 | 59.386 | 1.00 | 38.14 | O |
| ATOM | 16669 | N | GLN | I | 49 | 25.110 | 43.048 | 58.318 | 1.00 | 38.44 | N |
| ATOM | 16671 | CA | GLN | I | 49 | 25.578 | 43.471 | 56.996 | 1.00 | 38.25 | C |
| ATOM | 16673 | CB | GLN | I | 49 | 26.463 | 42.409 | 56.322 | 1.00 | 41.89 | C |
| ATOM | 16676 | CG | GLN | I | 49 | 26.777 | 42.582 | 54.811 | 1.00 | 55.60 | C |
| ATOM | 16679 | CD | GLN | I | 49 | 26.304 | 41.366 | 53.981 | 1.00 | 80.77 | C |
| ATOM | 16680 | OE1 | GLN | I | 49 | 26.855 | 40.257 | 54.121 | 1.00 | 88.22 | O |
| ATOM | 16681 | NE2 | GLN | I | 49 | 25.250 | 41.556 | 53.166 | 1.00 | 63.73 | N |
| ATOM | 16684 | C | GLN | I | 49 | 24.384 | 43.839 | 56.136 | 1.00 | 38.15 | C |
| ATOM | 16685 | O | GLN | I | 49 | 23.482 | 43.024 | 55.914 | 1.00 | 38.23 | O |
| ATOM | 16686 | N | LEU | I | 50 | 24.389 | 45.091 | 55.690 | 1.00 | 37.07 | N |
| ATOM | 16688 | CA | LEU | I | 50 | 23.352 | 45.628 | 54.823 | 1.00 | 44.07 | C |
| ATOM | 16690 | CB | LEU | I | 50 | 22.881 | 46.984 | 55.373 | 1.00 | 46.83 | C |
| ATOM | 16693 | CG | LEU | I | 50 | 22.324 | 47.982 | 54.355 | 1.00 | 48.68 | C |
| ATOM | 16695 | CD1 | LEU | I | 50 | 20.940 | 47.515 | 53.953 | 1.00 | 34.31 | C |
| ATOM | 16699 | CD2 | LEU | I | 50 | 22.296 | 49.393 | 54.931 | 1.00 | 52.38 | C |
| ATOM | 16703 | C | LEU | I | 50 | 23.866 | 45.800 | 53.400 | 1.00 | 45.80 | C |
| ATOM | 16704 | O | LEU | I | 50 | 24.961 | 46.287 | 53.174 | 1.00 | 46.71 | O |
| ATOM | 16705 | N | THR | I | 51 | 23.006 | 45.519 | 52.434 | 1.00 | 56.28 | N |
| ATOM | 16707 | CA | THR | I | 51 | 23.352 | 45.650 | 51.022 | 1.00 | 54.30 | C |
| ATOM | 16709 | CB | THR | I | 51 | 23.604 | 44.275 | 50.399 | 1.00 | 48.41 | C |
| ATOM | 16711 | OG1 | THR | I | 51 | 24.590 | 43.590 | 51.175 | 1.00 | 62.50 | O |
| ATOM | 16713 | CG2 | THR | I | 51 | 24.236 | 44.412 | 49.032 | 1.00 | 55.28 | C |
| ATOM | 16717 | C | THR | I | 51 | 22.205 | 46.317 | 50.296 | 1.00 | 50.38 | C |
| ATOM | 16718 | O | THR | I | 51 | 21.063 | 45.873 | 50.397 | 1.00 | 46.90 | O |
| ATOM | 16719 | N | LEU | I | 52 | 22.544 | 47.390 | 49.587 | 1.00 | 49.30 | N |
| ATOM | 16721 | CA | LEU | I | 52 | 21.640 | 48.079 | 48.674 | 1.00 | 42.73 | C |
| ATOM | 16723 | CB | LEU | I | 52 | 21.218 | 49.416 | 49.298 | 1.00 | 40.50 | C |
| ATOM | 16726 | CG | LEU | I | 52 | 20.489 | 49.330 | 50.627 | 1.00 | 34.75 | C |
| ATOM | 16728 | CD1 | LEU | I | 52 | 20.235 | 50.684 | 51.246 | 1.00 | 50.76 | C |
| ATOM | 16732 | CD2 | LEU | I | 52 | 19.166 | 48.645 | 50.472 | 1.00 | 30.66 | C |
| ATOM | 16736 | C | LEU | I | 52 | 22.355 | 48.340 | 47.343 | 1.00 | 41.04 | C |
| ATOM | 16737 | O | LEU | I | 52 | 23.318 | 49.122 | 47.265 | 1.00 | 37.76 | O |
| ATOM | 16738 | N | ARG | I | 53 | 21.843 | 47.698 | 46.300 | 1.00 | 39.58 | N |
| ATOM | 16740 | CA | ARG | I | 53 | 22.359 | 47.842 | 44.933 | 1.00 | 38.45 | C |
| ATOM | 16742 | CB | ARG | I | 53 | 21.878 | 46.668 | 44.078 | 1.00 | 34.69 | C |
| ATOM | 16745 | CG | ARG | I | 53 | 22.349 | 45.364 | 44.576 | 1.00 | 41.48 | C |
| ATOM | 16748 | CD | ARG | I | 53 | 22.123 | 44.155 | 43.698 | 1.00 | 46.48 | C |
| ATOM | 16751 | NE | ARG | I | 53 | 22.297 | 43.054 | 44.641 | 1.00 | 65.59 | N |
| ATOM | 16753 | CZ | ARG | I | 53 | 21.287 | 42.348 | 45.111 | 1.00 | 75.03 | C |
| ATOM | 16754 | NH1 | ARG | I | 53 | 20.070 | 42.604 | 44.576 | 1.00 | 45.63 | N |
| ATOM | 16757 | NH2 | ARG | I | 53 | 21.518 | 41.425 | 46.076 | 1.00 | 67.29 | N |
| ATOM | 16760 | C | ARG | I | 53 | 21.864 | 49.099 | 44.222 | 1.00 | 38.36 | C |
| ATOM | 16761 | O | ARG | I | 53 | 20.713 | 49.479 | 44.338 | 1.00 | 39.90 | O |
| ATOM | 16762 | N | SER | I | 54 | 22.690 | 49.660 | 43.358 | 1.00 | 42.08 | N |
| ATOM | 16764 | CA | SER | I | 54 | 22.307 | 50.893 | 42.710 | 1.00 | 46.28 | C |
| ATOM | 16766 | CB | SER | I | 54 | 23.514 | 51.515 | 42.044 | 1.00 | 46.20 | C |
| ATOM | 16769 | OG | SER | I | 54 | 24.360 | 50.498 | 41.574 | 1.00 | 47.82 | O |
| ATOM | 16771 | C | SER | I | 54 | 21.218 | 50.676 | 41.688 | 1.00 | 45.75 | C |
| ATOM | 16772 | O | SER | I | 54 | 20.452 | 51.616 | 41.417 | 1.00 | 48.55 | O |
| ATOM | 16773 | N | GLU | I | 55 | 21.148 | 49.465 | 41.131 | 1.00 | 45.65 | N |
| ATOM | 16775 | CA | GLU | I | 55 | 20.077 | 49.121 | 40.173 | 1.00 | 50.20 | C |
| ATOM | 16777 | CB | GLU | I | 55 | 20.181 | 47.675 | 39.649 | 1.00 | 50.91 | C |
| ATOM | 16780 | CG | GLU | I | 55 | 21.541 | 47.306 | 39.078 | 1.00 | 64.79 | C |
| ATOM | 16783 | CD | GLU | I | 55 | 22.395 | 46.577 | 40.096 | 1.00 | 89.16 | C |
| ATOM | 16784 | OE1 | GLU | I | 55 | 22.058 | 45.397 | 40.368 | 1.00 | 110.44 | O |
| ATOM | 16785 | OE2 | GLU | I | 55 | 23.356 | 47.189 | 40.641 | 1.00 | 60.30 | O |
| ATOM | 16786 | C | GLU | I | 55 | 18.715 | 49.352 | 40.820 | 1.00 | 43.07 | C |
| ATOM | 16787 | O | GLU | I | 55 | 17.763 | 49.783 | 40.191 | 1.00 | 48.89 | O |
| ATOM | 16788 | N | GLY | I | 56 | 18.670 | 49.174 | 42.129 | 1.00 | 45.07 | N |
| ATOM | 16790 | CA | GLY | I | 56 | 17.423 | 49.274 | 42.861 | 1.00 | 46.42 | C |
| ATOM | 16793 | C | GLY | I | 56 | 17.013 | 50.649 | 43.337 | 1.00 | 40.35 | C |
| ATOM | 16794 | O | GLY | I | 56 | 15.952 | 50.765 | 43.956 | 1.00 | 50.79 | O |
| ATOM | 16795 | N | PHE | I | 57 | 17.812 | 51.669 | 43.041 | 1.00 | 41.32 | N |
| ATOM | 16797 | CA | PHE | I | 57 | 17.347 | 53.058 | 43.090 | 1.00 | 43.69 | C |
| ATOM | 16799 | CB | PHE | I | 57 | 18.425 | 53.999 | 43.609 | 1.00 | 43.54 | C |
| ATOM | 16802 | CG | PHE | I | 57 | 18.944 | 53.678 | 44.967 | 1.00 | 37.27 | C |
| ATOM | 16803 | CD1 | PHE | I | 57 | 19.788 | 52.594 | 45.182 | 1.00 | 34.49 | C |
| ATOM | 16805 | CE1 | PHE | I | 57 | 20.285 | 52.341 | 46.448 | 1.00 | 41.63 | C |
| ATOM | 16807 | CZ | PHE | I | 57 | 20.005 | 53.219 | 47.500 | 1.00 | 34.91 | C |
| ATOM | 16809 | CE2 | PHE | I | 57 | 19.205 | 54.324 | 47.283 | 1.00 | 31.47 | C |
| ATOM | 16811 | CD2 | PHE | I | 57 | 18.716 | 54.571 | 46.005 | 1.00 | 45.80 | C |
| ATOM | 16813 | C | PHE | I | 57 | 16.942 | 53.617 | 41.726 | 1.00 | 37.95 | C |
| ATOM | 16814 | O | PHE | I | 57 | 17.577 | 53.392 | 40.724 | 1.00 | 45.08 | O |
| ATOM | 16815 | N | ASP | I | 58 | 15.922 | 54.444 | 41.734 | 1.00 | 41.30 | N |
| ATOM | 16817 | CA | ASP | I | 58 | 15.504 | 55.164 | 40.556 | 1.00 | 45.77 | C |
| ATOM | 16819 | CB | ASP | I | 58 | 14.537 | 56.257 | 41.001 | 1.00 | 46.76 | C |
| ATOM | 16822 | CG | ASP | I | 58 | 13.788 | 56.886 | 39.857 | 1.00 | 51.10 | C |
| ATOM | 16823 | OD1 | ASP | I | 58 | 13.500 | 56.111 | 38.910 | 1.00 | 50.63 | O |
| ATOM | 16824 | OD2 | ASP | I | 58 | 13.433 | 58.100 | 39.882 | 1.00 | 54.47 | O |
| ATOM | 16825 | C | ASP | I | 58 | 16.707 | 55.798 | 39.898 | 1.00 | 44.43 | C |
| ATOM | 16826 | O | ASP | I | 58 | 16.852 | 55.764 | 38.688 | 1.00 | 45.47 | O |
| ATOM | 16827 | N | THR | I | 59 | 17.542 | 56.430 | 40.709 | 1.00 | 49.79 | N |
| ATOM | 16829 | CA | THR | I | 59 | 18.691 | 57.180 | 40.213 | 1.00 | 47.14 | C |
| ATOM | 16831 | CB | THR | I | 59 | 18.403 | 58.657 | 40.281 | 1.00 | 44.33 | C |
| ATOM | 16833 | OG1 | THR | I | 59 | 17.515 | 58.997 | 39.224 | 1.00 | 46.17 | O |
| ATOM | 16835 | CG2 | THR | I | 59 | 19.658 | 59.468 | 40.040 | 1.00 | 64.91 | C |
| ATOM | 16839 | C | THR | I | 59 | 19.838 | 56.938 | 41.157 | 1.00 | 47.13 | C |
| ATOM | 16840 | O | THR | I | 59 | 19.666 | 57.001 | 42.373 | 1.00 | 50.67 | O |
| ATOM | 16841 | N | TYR | I | 60 | 21.028 | 56.800 | 40.606 | 1.00 | 42.57 | N |
| ATOM | 16843 | CA | TYR | I | 60 | 22.165 | 56.594 | 41.473 | 1.00 | 43.40 | C |
| ATOM | 16845 | CB | TYR | I | 60 | 22.226 | 55.118 | 41.818 | 1.00 | 39.13 | C |
| ATOM | 16848 | CG | TYR | I | 60 | 23.202 | 54.795 | 42.891 | 1.00 | 38.14 | C |
| ATOM | 16849 | CD1 | TYR | I | 60 | 22.763 | 54.596 | 44.201 | 1.00 | 64.21 | C |
| ATOM | 16851 | CE1 | TYR | I | 60 | 23.643 | 54.290 | 45.227 | 1.00 | 46.94 | C |
| ATOM | 16853 | CZ | TYR | I | 60 | 24.972 | 54.196 | 44.945 | 1.00 | 38.38 | C |
| ATOM | 16854 | OH | TYR | I | 60 | 25.824 | 53.867 | 45.956 | 1.00 | 40.57 | O |
| ATOM | 16856 | CE2 | TYR | I | 60 | 25.451 | 54.426 | 43.659 | 1.00 | 45.61 | C |
| ATOM | 16858 | CD2 | TYR | I | 60 | 24.559 | 54.741 | 42.634 | 1.00 | 37.41 | C |
| ATOM | 16860 | C | TYR | I | 60 | 23.438 | 57.005 | 40.789 | 1.00 | 38.22 | C |
| ATOM | 16861 | O | TYR | I | 60 | 23.751 | 56.480 | 39.746 | 1.00 | 51.02 | O |
| ATOM | 16862 | N | ARG | I | 61 | 24.154 | 57.935 | 41.395 | 1.00 | 51.44 | N |
| ATOM | 16864 | CA | ARG | I | 61 | 25.415 | 58.487 | 40.880 | 1.00 | 57.66 | C |
| ATOM | 16866 | CB | ARG | I | 61 | 25.207 | 59.953 | 40.432 | 1.00 | 55.14 | C |
| ATOM | 16869 | CG | ARG | I | 61 | 26.478 | 60.772 | 40.175 | 1.00 | 67.71 | C |
| ATOM | 16872 | CD | ARG | I | 61 | 26.447 | 61.655 | 38.906 | 1.00 | 87.29 | C |
| ATOM | 16875 | NE | ARG | I | 61 | 26.875 | 63.037 | 39.155 | 1.00 | 92.77 | N |
| ATOM | 16877 | CZ | ARG | I | 61 | 26.058 | 64.005 | 39.578 | 1.00 | 95.28 | C |
| ATOM | 16878 | NH1 | ARG | I | 61 | 24.779 | 63.703 | 39.811 | 1.00 | 107.21 | N |
| ATOM | 16881 | NH2 | ARG | I | 61 | 26.494 | 65.260 | 39.780 | 1.00 | 46.31 | N |
| ATOM | 16884 | C | ARG | I | 61 | 26.389 | 58.444 | 42.057 | 1.00 | 50.88 | C |
| ATOM | 16885 | O | ARG | I | 61 | 26.031 | 58.874 | 43.137 | 1.00 | 50.80 | O |
| ATOM | 16886 | N | CYS | I | 62 | 27.577 | 57.896 | 41.856 | 1.00 | 49.59 | N |
| ATOM | 16888 | CA | CYS | I | 62 | 28.677 | 57.996 | 42.804 | 1.00 | 50.20 | C |
| ATOM | 16890 | CB | CYS | I | 62 | 28.745 | 56.782 | 43.723 | 1.00 | 51.50 | C |
| ATOM | 16893 | SG | CYS | I | 62 | 30.125 | 56.871 | 44.923 | 1.00 | 66.60 | S |
| ATOM | 16894 | C | CYS | I | 62 | 29.992 | 58.037 | 42.035 | 1.00 | 55.70 | C |
| ATOM | 16895 | O | CYS | I | 62 | 30.431 | 56.995 | 41.529 | 1.00 | 44.40 | O |
| ATOM | 16896 | N | ASP | I | 63 | 30.609 | 59.226 | 42.043 | 1.00 | 60.64 | N |
| ATOM | 16898 | CA | ASP | I | 63 | 31.810 | 59.604 | 41.294 | 1.00 | 59.18 | C |
| ATOM | 16900 | CB | ASP | I | 63 | 31.886 | 61.123 | 41.214 | 1.00 | 58.71 | C |
| ATOM | 16903 | CG | ASP | I | 63 | 30.704 | 61.737 | 40.502 | 1.00 | 80.50 | C |
| ATOM | 16904 | OD1 | ASP | I | 63 | 30.077 | 61.017 | 39.685 | 1.00 | 105.56 | O |
| ATOM | 16905 | OD2 | ASP | I | 63 | 30.356 | 62.931 | 40.685 | 1.00 | 75.77 | O |
| ATOM | 16906 | C | ASP | I | 63 | 33.097 | 59.213 | 41.998 | 1.00 | 61.69 | C |
| ATOM | 16907 | O | ASP | I | 63 | 34.138 | 59.120 | 41.357 | 1.00 | 66.56 | O |
| ATOM | 16908 | N | ARG | I | 64 | 33.054 | 59.158 | 43.327 | 1.00 | 67.52 | N |
| ATOM | 16910 | CA | ARG | I | 64 | 34.188 | 58.738 | 44.162 | 1.00 | 69.44 | C |
| ATOM | 16912 | CB | ARG | I | 64 | 35.181 | 59.873 | 44.453 | 1.00 | 63.87 | C |
| ATOM | 16915 | CG | ARG | I | 64 | 36.442 | 59.779 | 43.547 | 1.00 | 91.89 | C |
| ATOM | 16918 | CD | ARG | I | 64 | 37.806 | 59.830 | 44.255 | 1.00 | 102.49 | C |
| ATOM | 16921 | NE | ARG | I | 64 | 37.822 | 60.955 | 45.184 | 1.00 | 116.34 | N |
| ATOM | 16923 | CZ | ARG | I | 64 | 37.773 | 62.232 | 44.815 | 1.00 | 113.06 | C |
| ATOM | 16924 | NH1 | ARG | I | 64 | 37.795 | 62.564 | 43.529 | 1.00 | 98.75 | N |
| ATOM | 16927 | NH2 | ARG | I | 64 | 37.724 | 63.184 | 45.739 | 1.00 | 119.12 | N |
| ATOM | 16930 | C | ARG | I | 64 | 33.738 | 58.077 | 45.457 | 1.00 | 65.96 | C |
| ATOM | 16931 | O | ARG | I | 64 | 32.881 | 58.579 | 46.182 | 1.00 | 70.45 | O |
| ATOM | 16932 | N | ASN | I | 65 | 34.277 | 56.895 | 45.708 | 1.00 | 57.60 | N |
| ATOM | 16934 | CA | ASN | I | 65 | 33.827 | 56.140 | 46.835 | 1.00 | 54.78 | C |
| ATOM | 16936 | CB | ASN | I | 65 | 34.779 | 54.991 | 47.086 | 1.00 | 56.54 | C |
| ATOM | 16939 | CG | ASN | I | 65 | 34.703 | 53.937 | 46.013 | 1.00 | 55.16 | C |
| ATOM | 16940 | OD1 | ASN | I | 65 | 35.494 | 53.010 | 46.028 | 1.00 | 68.55 | O |
| ATOM | 16941 | ND2 | ASN | I | 65 | 33.758 | 54.062 | 45.081 | 1.00 | 55.21 | N |
| ATOM | 16944 | C | ASN | I | 65 | 33.805 | 57.077 | 48.031 | 1.00 | 62.17 | C |
| ATOM | 16945 | O | ASN | I | 65 | 34.586 | 58.019 | 48.077 | 1.00 | 55.02 | O |
| ATOM | 16946 | N | LEU | I | 66 | 32.880 | 56.820 | 48.960 | 1.00 | 65.56 | N |
| ATOM | 16948 | CA | LEU | I | 66 | 32.792 | 57.521 | 50.239 | 1.00 | 62.08 | C |
| ATOM | 16950 | CB | LEU | I | 66 | 31.476 | 58.275 | 50.334 | 1.00 | 62.10 | C |
| ATOM | 16953 | CG | LEU | I | 66 | 31.343 | 59.613 | 49.635 | 1.00 | 64.92 | C |
| ATOM | 16955 | CD1 | LEU | I | 66 | 31.358 | 59.324 | 48.151 | 1.00 | 83.61 | C |
| ATOM | 16959 | CD2 | LEU | I | 66 | 30.035 | 60.259 | 50.044 | 1.00 | 67.12 | C |
| ATOM | 16963 | C | LEU | I | 66 | 32.758 | 56.526 | 51.385 | 1.00 | 56.23 | C |
| ATOM | 16964 | O | LEU | I | 66 | 32.165 | 55.457 | 51.259 | 1.00 | 47.63 | O |
| ATOM | 16965 | N | ALA | I | 67 | 33.239 | 56.958 | 52.542 | 1.00 | 48.93 | N |
| ATOM | 16967 | CA | ALA | I | 67 | 32.919 | 56.258 | 53.773 | 1.00 | 55.75 | C |
| ATOM | 16969 | CB | ALA | I | 67 | 34.169 | 55.601 | 54.398 | 1.00 | 48.28 | C |
| ATOM | 16973 | C | ALA | I | 67 | 32.316 | 57.240 | 54.742 | 1.00 | 57.39 | C |
| ATOM | 16974 | O | ALA | I | 67 | 33.014 | 58.171 | 55.173 | 1.00 | 68.38 | O |
| ATOM | 16975 | N | MET | I | 68 | 31.051 | 57.022 | 55.094 | 1.00 | 50.48 | N |
| ATOM | 16977 | CA | MET | I | 68 | 30.346 | 58.019 | 55.876 | 1.00 | 50.54 | C |
| ATOM | 16979 | CB | MET | I | 68 | 29.036 | 58.379 | 55.212 | 1.00 | 46.54 | C |
| ATOM | 16982 | CG | MET | I | 68 | 29.178 | 59.050 | 53.892 | 1.00 | 45.80 | C |
| ATOM | 16985 | SD | MET | I | 68 | 27.624 | 59.851 | 53.357 | 1.00 | 46.16 | S |
| ATOM | 16986 | CE | MET | I | 68 | 26.460 | 58.534 | 53.161 | 1.00 | 49.12 | C |
| ATOM | 16990 | C | MET | I | 68 | 30.100 | 57.537 | 57.299 | 1.00 | 50.10 | C |
| ATOM | 16991 | O | MET | I | 68 | 29.380 | 56.567 | 57.502 | 1.00 | 47.53 | O |
| ATOM | 16992 | N | GLY | I | 69 | 30.664 | 58.229 | 58.288 | 1.00 | 52.06 | N |
| ATOM | 16994 | CA | GLY | I | 69 | 30.289 | 57.985 | 59.678 | 1.00 | 50.78 | C |
| ATOM | 16997 | C | GLY | I | 69 | 28.936 | 58.541 | 60.134 | 1.00 | 45.15 | C |
| ATOM | 16998 | O | GLY | I | 69 | 28.682 | 59.753 | 60.106 | 1.00 | 45.71 | O |
| ATOM | 16999 | N | VAL | I | 70 | 28.043 | 57.647 | 60.537 | 1.00 | 39.51 | N |
| ATOM | 17001 | CA | VAL | I | 70 | 26.633 | 57.997 | 60.699 | 1.00 | 37.84 | C |
| ATOM | 17003 | CB | VAL | I | 70 | 25.745 | 57.296 | 59.671 | 1.00 | 40.92 | C |
| ATOM | 17005 | CG1 | VAL | I | 70 | 24.268 | 57.376 | 60.084 | 1.00 | 41.24 | C |
| ATOM | 17009 | CG2 | VAL | I | 70 | 25.939 | 57.925 | 58.299 | 1.00 | 48.46 | C |
| ATOM | 17013 | C | VAL | I | 70 | 26.169 | 57.578 | 62.097 | 1.00 | 40.75 | C |
| ATOM | 17014 | O | VAL | I | 70 | 26.126 | 56.401 | 62.415 | 1.00 | 42.46 | O |
| ATOM | 17015 | N | ASN | I | 71 | 25.887 | 58.542 | 62.965 | 1.00 | 40.21 | N |
| ATOM | 17017 | CA | ASN | I | 71 | 25.195 | 58.211 | 64.192 | 1.00 | 39.21 | C |
| ATOM | 17019 | CB | ASN | I | 71 | 25.074 | 59.426 | 65.129 | 1.00 | 36.62 | C |
| ATOM | 17022 | CG | ASN | I | 71 | 24.355 | 59.074 | 66.441 | 1.00 | 44.27 | C |
| ATOM | 17023 | OD1 | ASN | I | 71 | 23.230 | 58.556 | 66.455 | 1.00 | 36.74 | O |
| ATOM | 17024 | ND2 | ASN | I | 71 | 25.025 | 59.324 | 67.548 | 1.00 | 43.18 | N |
| ATOM | 17027 | C | ASN | I | 71 | 23.828 | 57.735 | 63.774 | 1.00 | 28.16 | C |
| ATOM | 17028 | O | ASN | I | 71 | 23.036 | 58.520 | 63.293 | 1.00 | 38.01 | O |
| ATOM | 17029 | N | LEU | I | 72 | 23.554 | 56.458 | 63.957 | 1.00 | 32.75 | N |
| ATOM | 17031 | CA | LEU | I | 72 | 22.360 | 55.818 | 63.386 | 1.00 | 33.22 | C |
| ATOM | 17033 | CB | LEU | I | 72 | 22.443 | 54.281 | 63.467 | 1.00 | 28.09 | C |
| ATOM | 17036 | CG | LEU | I | 72 | 23.544 | 53.587 | 62.669 | 1.00 | 25.73 | C |
| ATOM | 17038 | CD1 | LEU | I | 72 | 23.203 | 52.130 | 62.499 | 1.00 | 48.05 | C |
| ATOM | 17042 | CD2 | LEU | I | 72 | 23.646 | 54.229 | 61.314 | 1.00 | 41.60 | C |
| ATOM | 17046 | C | LEU | I | 72 | 21.104 | 56.270 | 64.101 | 1.00 | 34.06 | C |
| ATOM | 17047 | O | LEU | I | 72 | 20.035 | 56.259 | 63.518 | 1.00 | 38.34 | O |
| ATOM | 17048 | N | THR | I | 73 | 21.245 | 56.715 | 65.344 | 1.00 | 39.37 | N |
| ATOM | 17050 | CA | THR | I | 73 | 20.136 | 57.335 | 66.065 | 1.00 | 42.39 | C |
| ATOM | 17052 | CB | THR | I | 73 | 20.509 | 57.595 | 67.547 | 1.00 | 42.79 | C |
| ATOM | 17054 | OG1 | THR | I | 73 | 20.642 | 56.361 | 68.269 | 1.00 | 40.48 | O |
| ATOM | 17056 | CG2 | THR | I | 73 | 19.384 | 58.276 | 68.260 | 1.00 | 43.42 | C |
| ATOM | 17060 | C | THR | I | 73 | 19.751 | 58.659 | 65.429 | 1.00 | 36.37 | C |
| ATOM | 17061 | O | THR | I | 73 | 18.575 | 58.945 | 65.256 | 1.00 | 38.17 | O |
| ATOM | 17062 | N | SER | I | 74 | 20.756 | 59.456 | 65.102 | 1.00 | 36.02 | N |
| ATOM | 17064 | CA | SER | I | 74 | 20.564 | 60.744 | 64.437 | 1.00 | 40.59 | C |
| ATOM | 17066 | CB | SER | I | 74 | 21.918 | 61.479 | 64.292 | 1.00 | 40.24 | C |
| ATOM | 17069 | OG | SER | I | 74 | 22.401 | 61.983 | 65.565 | 1.00 | 33.74 | O |
| ATOM | 17071 | C | SER | I | 74 | 19.839 | 60.594 | 63.101 | 1.00 | 35.23 | C |
| ATOM | 17072 | O | SER | I | 74 | 18.909 | 61.333 | 62.778 | 1.00 | 42.87 | O |
| ATOM | 17073 | N | MET | I | 75 | 20.198 | 59.561 | 62.364 | 1.00 | 35.45 | N |
| ATOM | 17075 | CA | MET | I | 75 | 19.707 | 59.404 | 61.008 | 1.00 | 40.37 | C |
| ATOM | 17077 | CB | MET | I | 75 | 20.637 | 58.479 | 60.210 | 1.00 | 43.26 | C |
| ATOM | 17080 | CG | MET | I | 75 | 20.260 | 58.156 | 58.763 | 1.00 | 36.59 | C |
| ATOM | 17083 | SD | MET | I | 75 | 21.422 | 57.015 | 57.852 | 1.00 | 46.00 | S |
| ATOM | 17084 | CE | MET | I | 75 | 20.718 | 57.254 | 56.205 | 1.00 | 37.37 | C |
| ATOM | 17088 | C | MET | I | 75 | 18.309 | 58.831 | 61.156 | 1.00 | 38.98 | C |
| ATOM | 17089 | O | MET | I | 75 | 17.388 | 59.276 | 60.491 | 1.00 | 43.17 | O |
| ATOM | 17090 | N | SER | I | 76 | 18.118 | 57.926 | 62.100 | 1.00 | 33.84 | N |
| ATOM | 17092 | CA | SER | I | 76 | 16.758 | 57.534 | 62.487 | 1.00 | 41.25 | C |
| ATOM | 17094 | CB | SER | I | 76 | 16.777 | 56.622 | 63.725 | 1.00 | 41.54 | C |
| ATOM | 17097 | OG | SER | I | 76 | 15.520 | 55.972 | 63.887 | 1.00 | 60.85 | O |
| ATOM | 17099 | C | SER | I | 76 | 15.750 | 58.690 | 62.709 | 1.00 | 45.61 | C |
| ATOM | 17100 | O | SER | I | 76 | 14.560 | 58.583 | 62.306 | 1.00 | 36.72 | O |
| ATOM | 17101 | N | LYS | I | 77 | 16.209 | 59.744 | 63.397 | 1.00 | 39.29 | N |
| ATOM | 17103 | CA | LYS | I | 77 | 15.304 | 60.732 | 63.953 | 1.00 | 34.95 | C |
| ATOM | 17105 | CB | LYS | I | 77 | 15.963 | 61.700 | 64.943 | 1.00 | 41.47 | C |
| ATOM | 17108 | CG | LYS | I | 77 | 16.021 | 61.231 | 66.425 | 1.00 | 49.48 | C |
| ATOM | 17111 | CD | LYS | I | 77 | 16.285 | 62.440 | 67.382 | 1.00 | 80.50 | C |
| ATOM | 17114 | CE | LYS | I | 77 | 17.041 | 62.120 | 68.688 | 1.00 | 86.82 | C |
| ATOM | 17117 | NZ | LYS | I | 77 | 16.221 | 61.370 | 69.691 | 1.00 | 89.47 | N |
| ATOM | 17121 | C | LYS | I | 77 | 14.901 | 61.460 | 62.714 | 1.00 | 33.73 | C |
| ATOM | 17122 | O | LYS | I | 77 | 13.729 | 61.647 | 62.470 | 1.00 | 36.49 | O |
| ATOM | 17123 | N | ILE | I | 78 | 15.870 | 61.754 | 61.862 | 1.00 | 37.21 | N |
| ATOM | 17125 | CA | ILE | I | 78 | 15.554 | 62.363 | 60.579 | 1.00 | 35.28 | C |
| ATOM | 17127 | CB | ILE | I | 78 | 16.872 | 62.637 | 59.892 | 1.00 | 38.15 | C |
| ATOM | 17129 | CG1 | ILE | I | 78 | 17.707 | 63.657 | 60.677 | 1.00 | 36.96 | C |
| ATOM | 17132 | CD1 | ILE | I | 78 | 18.832 | 64.217 | 59.804 | 1.00 | 38.36 | C |
| ATOM | 17136 | CG2 | ILE | I | 78 | 16.643 | 63.146 | 58.462 | 1.00 | 48.24 | C |
| ATOM | 17140 | C | ILE | I | 78 | 14.652 | 61.512 | 59.655 | 1.00 | 37.56 | C |
| ATOM | 17141 | O | ILE | I | 78 | 13.736 | 61.987 | 58.980 | 1.00 | 35.44 | O |
| ATOM | 17142 | N | LEU | I | 79 | 14.922 | 60.220 | 59.596 | 1.00 | 43.14 | N |
| ATOM | 17144 | CA | LEU | I | 79 | 14.180 | 59.379 | 58.680 | 1.00 | 42.00 | C |
| ATOM | 17146 | CB | LEU | I | 79 | 14.788 | 57.980 | 58.631 | 1.00 | 41.74 | C |
| ATOM | 17149 | CG | LEU | I | 79 | 15.545 | 57.591 | 57.361 | 1.00 | 39.69 | C |
| ATOM | 17151 | CD1 | LEU | I | 79 | 14.678 | 57.883 | 56.179 | 1.00 | 47.79 | C |
| ATOM | 17155 | CD2 | LEU | I | 79 | 16.838 | 58.348 | 57.195 | 1.00 | 57.50 | C |
| ATOM | 17159 | C | LEU | I | 79 | 12.733 | 59.333 | 59.166 | 1.00 | 42.76 | C |
| ATOM | 17160 | O | LEU | I | 79 | 11.824 | 59.039 | 58.405 | 1.00 | 36.43 | O |
| ATOM | 17161 | N | LYS | I | 80 | 12.499 | 59.640 | 60.432 | 1.00 | 42.64 | N |
| ATOM | 17163 | CA | LYS | I | 80 | 11.150 | 59.478 | 60.962 | 1.00 | 42.97 | C |
| ATOM | 17165 | CB | LYS | I | 80 | 11.144 | 59.249 | 62.483 | 1.00 | 46.06 | C |
| ATOM | 17168 | CG | LYS | I | 80 | 11.214 | 57.759 | 62.907 | 1.00 | 59.95 | C |
| ATOM | 17171 | CD | LYS | I | 80 | 11.518 | 57.628 | 64.413 | 1.00 | 75.32 | C |
| ATOM | 17174 | CE | LYS | I | 80 | 11.417 | 56.193 | 64.957 | 1.00 | 89.93 | C |
| ATOM | 17177 | NZ | LYS | I | 80 | 10.009 | 55.685 | 65.142 | 1.00 | 87.96 | N |
| ATOM | 17181 | C | LYS | I | 80 | 10.365 | 60.714 | 60.571 | 1.00 | 40.90 | C |
| ATOM | 17182 | O | LYS | I | 80 | 9.134 | 60.696 | 60.583 | 1.00 | 39.58 | O |
| ATOM | 17183 | N | CYS | I | 81 | 11.067 | 61.747 | 60.116 | 1.00 | 42.03 | N |
| ATOM | 17185 | CA | CYS | I | 81 | 10.374 | 62.911 | 59.570 | 1.00 | 52.74 | C |
| ATOM | 17187 | CB | CYS | I | 81 | 11.266 | 64.136 | 59.581 | 1.00 | 59.08 | C |
| ATOM | 17190 | SG | CYS | I | 81 | 11.854 | 64.497 | 61.226 | 1.00 | 51.22 | S |
| ATOM | 17191 | C | CYS | I | 81 | 9.933 | 62.781 | 58.129 | 1.00 | 52.01 | C |
| ATOM | 17192 | O | CYS | I | 81 | 9.376 | 63.736 | 57.575 | 1.00 | 49.39 | O |
| ATOM | 17193 | N | ALA | I | 82 | 10.265 | 61.658 | 57.501 | 1.00 | 45.24 | N |
| ATOM | 17195 | CA | ALA | I | 82 | 9.768 | 61.421 | 56.166 | 1.00 | 39.48 | C |
| ATOM | 17197 | CB | ALA | I | 82 | 10.765 | 60.708 | 55.355 | 1.00 | 49.68 | C |
| ATOM | 17201 | C | ALA | I | 82 | 8.544 | 60.583 | 56.312 | 1.00 | 42.02 | C |
| ATOM | 17202 | O | ALA | I | 82 | 8.400 | 59.873 | 57.319 | 1.00 | 34.61 | O |
| ATOM | 17203 | N | GLY | I | 83 | 7.659 | 60.728 | 55.324 | 1.00 | 42.96 | N |
| ATOM | 17205 | CA | GLY | I | 83 | 6.345 | 60.098 | 55.350 | 1.00 | 44.88 | C |
| ATOM | 17208 | C | GLY | I | 83 | 6.484 | 58.802 | 54.592 | 1.00 | 41.70 | C |
| ATOM | 17209 | O | GLY | I | 83 | 7.563 | 58.574 | 54.082 | 1.00 | 49.20 | O |
| ATOM | 17210 | N | ASN | I | 84 | 5.484 | 57.928 | 54.569 | 1.00 | 46.52 | N |
| ATOM | 17212 | CA | ASN | I | 84 | 5.690 | 56.547 | 54.108 | 1.00 | 46.62 | C |
| ATOM | 17214 | CB | ASN | I | 84 | 4.651 | 55.616 | 54.734 | 1.00 | 49.11 | C |
| ATOM | 17217 | CG | ASN | I | 84 | 4.839 | 55.403 | 56.221 | 1.00 | 49.26 | C |
| ATOM | 17218 | OD1 | ASN | I | 84 | 5.931 | 55.090 | 56.685 | 1.00 | 38.84 | O |
| ATOM | 17219 | ND2 | ASN | I | 84 | 3.740 | 55.505 | 56.969 | 1.00 | 72.68 | N |
| ATOM | 17222 | C | ASN | I | 84 | 5.537 | 56.452 | 52.579 | 1.00 | 47.44 | C |
| ATOM | 17223 | O | ASN | I | 84 | 5.634 | 55.369 | 51.982 | 1.00 | 45.61 | O |
| ATOM | 17224 | N | GLU | I | 85 | 5.210 | 57.569 | 51.937 | 1.00 | 43.90 | N |
| ATOM | 17226 | CA | GLU | I | 85 | 5.095 | 57.542 | 50.490 | 1.00 | 40.60 | C |
| ATOM | 17228 | CB | GLU | I | 85 | 3.667 | 57.808 | 50.006 | 1.00 | 32.08 | C |
| ATOM | 17231 | CG | GLU | I | 85 | 2.653 | 56.866 | 50.632 | 1.00 | 42.21 | C |
| ATOM | 17234 | CD | GLU | I | 85 | 1.750 | 57.532 | 51.666 | 1.00 | 62.51 | C |
| ATOM | 17235 | OE1 | GLU | I | 85 | 2.048 | 57.417 | 52.870 | 1.00 | 58.63 | O |
| ATOM | 17236 | OE2 | GLU | I | 85 | 0.718 | 58.143 | 51.296 | 1.00 | 83.69 | O |
| ATOM | 17237 | C | GLU | I | 85 | 6.092 | 58.532 | 49.935 | 1.00 | 39.20 | C |
| ATOM | 17238 | O | GLU | I | 85 | 6.094 | 58.821 | 48.752 | 1.00 | 47.08 | O |
| ATOM | 17239 | N | ASP | I | 86 | 6.995 | 59.001 | 50.782 | 1.00 | 45.29 | N |
| ATOM | 17241 | CA | ASP | I | 86 | 7.882 | 60.092 | 50.382 | 1.00 | 41.92 | C |
| ATOM | 17243 | CB | ASP | I | 86 | 8.552 | 60.757 | 51.589 | 1.00 | 41.34 | C |
| ATOM | 17246 | CG | ASP | I | 86 | 7.677 | 61.782 | 52.276 | 1.00 | 47.00 | C |
| ATOM | 17247 | OD1 | ASP | I | 86 | 6.471 | 61.907 | 51.954 | 1.00 | 53.02 | O |
| ATOM | 17248 | OD2 | ASP | I | 86 | 8.128 | 62.452 | 53.220 | 1.00 | 49.03 | O |
| ATOM | 17249 | C | ASP | I | 86 | 8.971 | 59.545 | 49.500 | 1.00 | 40.02 | C |
| ATOM | 17250 | O | ASP | I | 86 | 9.574 | 58.492 | 49.761 | 1.00 | 45.31 | O |
| ATOM | 17251 | N | ILE | I | 87 | 9.291 | 60.337 | 48.495 | 1.00 | 41.48 | N |
| ATOM | 17253 | CA | ILE | I | 87 | 10.473 | 60.067 | 47.685 | 1.00 | 37.47 | C |
| ATOM | 17255 | CB | ILE | I | 87 | 10.339 | 60.733 | 46.301 | 1.00 | 35.53 | C |
| ATOM | 17257 | CG1 | ILE | I | 87 | 9.205 | 60.096 | 45.535 | 1.00 | 24.99 | C |
| ATOM | 17260 | CD1 | ILE | I | 87 | 7.996 | 61.036 | 45.240 | 1.00 | 46.38 | C |
| ATOM | 17264 | CG2 | ILE | I | 87 | 11.625 | 60.696 | 45.506 | 1.00 | 37.64 | C |
| ATOM | 17268 | C | ILE | I | 87 | 11.652 | 60.645 | 48.435 | 1.00 | 34.11 | C |
| ATOM | 17269 | O | ILE | I | 87 | 11.665 | 61.826 | 48.761 | 1.00 | 39.14 | O |
| ATOM | 17270 | N | ILE | I | 88 | 12.627 | 59.783 | 48.686 | 1.00 | 39.05 | N |
| ATOM | 17272 | CA | ILE | I | 88 | 13.783 | 60.092 | 49.509 | 1.00 | 43.85 | C |
| ATOM | 17274 | CB | ILE | I | 88 | 13.968 | 59.022 | 50.608 | 1.00 | 42.86 | C |
| ATOM | 17276 | CG1 | ILE | I | 88 | 12.628 | 58.873 | 51.347 | 1.00 | 39.32 | C |
| ATOM | 17279 | CD1 | ILE | I | 88 | 12.759 | 58.456 | 52.758 | 1.00 | 50.16 | C |
| ATOM | 17283 | CG2 | ILE | I | 88 | 15.150 | 59.423 | 51.518 | 1.00 | 29.26 | C |
| ATOM | 17287 | C | ILE | I | 88 | 15.029 | 60.184 | 48.664 | 1.00 | 39.41 | C |
| ATOM | 17288 | O | ILE | I | 88 | 15.511 | 59.180 | 48.188 | 1.00 | 41.69 | O |
| ATOM | 17289 | N | THR | I | 89 | 15.580 | 61.382 | 48.555 | 1.00 | 39.61 | N |
| ATOM | 17291 | CA | THR | I | 89 | 16.917 | 61.546 | 48.010 | 1.00 | 43.87 | C |
| ATOM | 17293 | CB | THR | I | 89 | 16.902 | 62.738 | 47.038 | 1.00 | 43.05 | C |
| ATOM | 17295 | OG1 | THR | I | 89 | 15.805 | 62.585 | 46.124 | 1.00 | 44.00 | O |
| ATOM | 17297 | CG2 | THR | I | 89 | 18.117 | 62.707 | 46.128 | 1.00 | 45.68 | C |
| ATOM | 17301 | C | THR | I | 89 | 18.016 | 61.710 | 49.060 | 1.00 | 43.95 | C |
| ATOM | 17302 | O | THR | I | 89 | 17.953 | 62.605 | 49.903 | 1.00 | 46.48 | O |
| ATOM | 17303 | N | LEU | I | 90 | 19.030 | 60.843 | 49.001 | 1.00 | 47.37 | N |
| ATOM | 17305 | CA | LEU | I | 90 | 20.307 | 61.054 | 49.709 | 1.00 | 42.27 | C |
| ATOM | 17307 | CB | LEU | I | 90 | 20.898 | 59.717 | 50.144 | 1.00 | 33.04 | C |
| ATOM | 17310 | CG | LEU | I | 90 | 19.995 | 58.787 | 50.969 | 1.00 | 40.66 | C |
| ATOM | 17312 | CD1 | LEU | I | 90 | 20.514 | 57.362 | 50.942 | 1.00 | 35.76 | C |
| ATOM | 17316 | CD2 | LEU | I | 90 | 19.897 | 59.206 | 52.415 | 1.00 | 52.42 | C |
| ATOM | 17320 | C | LEU | I | 90 | 21.339 | 61.797 | 48.857 | 1.00 | 42.26 | C |
| ATOM | 17321 | O | LEU | I | 90 | 21.624 | 61.412 | 47.742 | 1.00 | 50.19 | O |
| ATOM | 17322 | N | ARG | I | 91 | 21.947 | 62.854 | 49.362 | 1.00 | 42.02 | N |
| ATOM | 17324 | CA | ARG | I | 91 | 23.010 | 63.454 | 48.584 | 1.00 | 44.50 | C |
| ATOM | 17326 | CB | ARG | I | 91 | 22.468 | 64.676 | 47.859 | 1.00 | 46.66 | C |
| ATOM | 17329 | CG | ARG | I | 91 | 23.512 | 65.421 | 46.992 | 1.00 | 65.63 | C |
| ATOM | 17332 | CD | ARG | I | 91 | 22.991 | 66.745 | 46.407 | 1.00 | 95.33 | C |
| ATOM | 17335 | NE | ARG | I | 91 | 21.810 | 66.573 | 45.541 | 1.00 | 124.25 | N |
| ATOM | 17337 | CZ | ARG | I | 91 | 20.540 | 66.845 | 45.875 | 1.00 | 123.75 | C |
| ATOM | 17338 | NH1 | ARG | I | 91 | 20.228 | 67.336 | 47.069 | 1.00 | 121.01 | N |
| ATOM | 17341 | NH2 | ARG | I | 91 | 19.562 | 66.635 | 45.000 | 1.00 | 120.55 | N |
| ATOM | 17344 | C | ARG | I | 91 | 24.204 | 63.817 | 49.448 | 1.00 | 44.86 | C |
| ATOM | 17345 | O | ARG | I | 91 | 24.057 | 64.373 | 50.529 | 1.00 | 49.72 | O |
| ATOM | 17346 | N | ALA | I | 92 | 25.396 | 63.504 | 48.965 | 1.00 | 53.86 | N |
| ATOM | 17348 | CA | ALA | I | 92 | 26.641 | 63.896 | 49.630 | 1.00 | 55.30 | C |
| ATOM | 17350 | CB | ALA | I | 92 | 27.182 | 62.735 | 50.436 | 1.00 | 50.44 | C |
| ATOM | 17354 | C | ALA | I | 92 | 27.714 | 64.377 | 48.657 | 1.00 | 56.38 | C |
| ATOM | 17355 | O | ALA | I | 92 | 27.767 | 63.991 | 47.492 | 1.00 | 63.26 | O |
| ATOM | 17356 | N | GLU | I | 93 | 28.637 | 65.173 | 49.168 | 1.00 | 64.14 | N |
| ATOM | 17358 | CA | GLU | I | 93 | 29.687 | 65.745 | 48.344 | 1.00 | 59.91 | C |
| ATOM | 17360 | CB | GLU | I | 93 | 29.930 | 67.209 | 48.716 | 1.00 | 52.37 | C |
| ATOM | 17363 | CG | GLU | I | 93 | 28.777 | 68.155 | 48.344 | 1.00 | 49.76 | C |
| ATOM | 17366 | CD | GLU | I | 93 | 28.542 | 68.249 | 46.838 | 1.00 | 83.67 | C |
| ATOM | 17367 | OE1 | GLU | I | 93 | 29.537 | 68.429 | 46.088 | 1.00 | 79.53 | O |
| ATOM | 17368 | OE2 | GLU | I | 93 | 27.371 | 68.115 | 46.389 | 1.00 | 86.86 | O |
| ATOM | 17369 | C | GLU | I | 93 | 30.932 | 64.907 | 48.517 | 1.00 | 69.62 | C |
| ATOM | 17370 | O | GLU | I | 93 | 30.965 | 63.953 | 49.302 | 1.00 | 70.50 | O |
| ATOM | 17371 | N | ASP | I | 94 | 31.953 | 65.262 | 47.750 | 1.00 | 82.87 | N |
| ATOM | 17373 | CA | ASP | I | 94 | 33.122 | 64.410 | 47.606 | 1.00 | 90.63 | C |
| ATOM | 17375 | CB | ASP | I | 94 | 34.050 | 64.979 | 46.518 | 1.00 | 96.22 | C |
| ATOM | 17378 | CG | ASP | I | 94 | 33.386 | 65.010 | 45.128 | 1.00 | 108.43 | C |
| ATOM | 17379 | OD1 | ASP | I | 94 | 33.583 | 64.014 | 44.382 | 1.00 | 110.45 | O |
| ATOM | 17380 | OD2 | ASP | I | 94 | 32.646 | 65.957 | 44.720 | 1.00 | 93.65 | O |
| ATOM | 17381 | C | ASP | I | 94 | 33.806 | 64.296 | 48.966 | 1.00 | 88.84 | C |
| ATOM | 17382 | O | ASP | I | 94 | 34.279 | 63.222 | 49.320 | 1.00 | 86.84 | O |
| ATOM | 17383 | N | ASN | I | 95 | 33.780 | 65.382 | 49.740 | 1.00 | 91.14 | N |
| ATOM | 17385 | CA | ASN | I | 95 | 34.207 | 65.391 | 51.146 | 1.00 | 94.15 | C |
| ATOM | 17387 | CB | ASN | I | 95 | 34.836 | 66.735 | 51.479 | 1.00 | 94.75 | C |
| ATOM | 17390 | CG | ASN | I | 95 | 35.864 | 67.155 | 50.453 | 1.00 | 104.70 | C |
| ATOM | 17391 | OD1 | ASN | I | 95 | 35.995 | 68.338 | 50.131 | 1.00 | 109.77 | O |
| ATOM | 17392 | ND2 | ASN | I | 95 | 36.604 | 66.180 | 49.928 | 1.00 | 114.68 | N |
| ATOM | 17395 | C | ASN | I | 95 | 33.086 | 65.166 | 52.152 | 1.00 | 97.62 | C |
| ATOM | 17396 | O | ASN | I | 95 | 32.389 | 66.093 | 52.564 | 1.00 | 109.63 | O |
| ATOM | 17397 | N | ALA | I | 96 | 32.921 | 63.926 | 52.578 | 1.00 | 98.29 | N |
| ATOM | 17399 | CA | ALA | I | 96 | 32.030 | 63.615 | 53.691 | 1.00 | 94.60 | C |
| ATOM | 17401 | CB | ALA | I | 96 | 32.319 | 62.172 | 54.208 | 1.00 | 96.19 | C |
| ATOM | 17405 | C | ALA | I | 96 | 32.206 | 64.613 | 54.831 | 1.00 | 86.62 | C |
| ATOM | 17406 | O | ALA | I | 96 | 32.998 | 64.375 | 55.739 | 1.00 | 91.00 | O |
| ATOM | 17407 | N | ASP | I | 97 | 31.411 | 65.669 | 54.873 | 1.00 | 78.47 | N |
| ATOM | 17409 | CA | ASP | I | 97 | 31.181 | 66.306 | 56.168 | 1.00 | 78.98 | C |
| ATOM | 17411 | CB | ASP | I | 97 | 31.582 | 67.758 | 56.067 | 1.00 | 81.38 | C |
| ATOM | 17414 | CG | ASP | I | 97 | 33.024 | 67.934 | 56.410 | 1.00 | 93.78 | C |
| ATOM | 17415 | OD1 | ASP | I | 97 | 33.751 | 68.628 | 55.671 | 1.00 | 115.72 | O |
| ATOM | 17416 | OD2 | ASP | I | 97 | 33.517 | 67.322 | 57.379 | 1.00 | 104.51 | O |
| ATOM | 17417 | C | ASP | I | 97 | 29.793 | 66.131 | 56.808 | 1.00 | 72.31 | C |
| ATOM | 17418 | O | ASP | I | 97 | 29.657 | 66.004 | 58.017 | 1.00 | 66.57 | O |
| ATOM | 17419 | N | THR | I | 98 | 28.787 | 65.965 | 55.964 | 1.00 | 65.99 | N |
| ATOM | 17421 | CA | THR | I | 98 | 27.413 | 66.280 | 56.281 | 1.00 | 63.44 | C |
| ATOM | 17423 | CB | THR | I | 98 | 27.277 | 67.814 | 56.251 | 1.00 | 59.77 | C |
| ATOM | 17425 | OG1 | THR | I | 98 | 26.085 | 68.199 | 55.574 | 1.00 | 49.73 | O |
| ATOM | 17427 | CG2 | THR | I | 98 | 28.358 | 68.406 | 55.328 | 1.00 | 77.32 | C |
| ATOM | 17431 | C | THR | I | 98 | 26.713 | 65.570 | 55.113 | 1.00 | 59.77 | C |
| ATOM | 17432 | O | THR | I | 98 | 27.269 | 65.545 | 54.023 | 1.00 | 60.40 | O |
| ATOM | 17433 | N | LEU | I | 99 | 25.593 | 64.880 | 55.336 | 1.00 | 55.32 | N |
| ATOM | 17435 | CA | LEU | I | 99 | 24.916 | 64.167 | 54.236 | 1.00 | 50.55 | C |
| ATOM | 17437 | CB | LEU | I | 99 | 24.859 | 62.641 | 54.456 | 1.00 | 52.43 | C |
| ATOM | 17440 | CG | LEU | I | 99 | 23.642 | 61.862 | 53.927 | 1.00 | 40.09 | C |
| ATOM | 17442 | CD1 | LEU | I | 99 | 23.766 | 61.363 | 52.510 | 1.00 | 31.32 | C |
| ATOM | 17446 | CD2 | LEU | I | 99 | 23.402 | 60.682 | 54.784 | 1.00 | 43.78 | C |
| ATOM | 17450 | C | LEU | I | 99 | 23.505 | 64.690 | 54.178 | 1.00 | 40.67 | C |
| ATOM | 17451 | O | LEU | I | 99 | 22.875 | 64.768 | 55.220 | 1.00 | 42.92 | O |
| ATOM | 17452 | N | ALA | I | 100 | 23.042 | 65.067 | 52.989 | 1.00 | 38.24 | N |
| ATOM | 17454 | CA | ALA | I | 100 | 21.662 | 65.527 | 52.779 | 1.00 | 42.15 | C |
| ATOM | 17456 | CB | ALA | I | 100 | 21.574 | 66.439 | 51.563 | 1.00 | 31.69 | C |
| ATOM | 17460 | C | ALA | I | 100 | 20.632 | 64.402 | 52.636 | 1.00 | 44.11 | C |
| ATOM | 17461 | O | ALA | I | 100 | 20.903 | 63.309 | 52.154 | 1.00 | 46.46 | O |
| ATOM | 17462 | N | LEU | I | 101 | 19.409 | 64.757 | 52.970 | 1.00 | 43.87 | N |
| ATOM | 17464 | CA | LEU | I | 101 | 18.284 | 63.870 | 52.889 | 1.00 | 43.73 | C |
| ATOM | 17466 | CB | LEU | I | 101 | 18.039 | 63.308 | 54.271 | 1.00 | 42.11 | C |
| ATOM | 17469 | CG | LEU | I | 101 | 19.088 | 62.237 | 54.531 | 1.00 | 48.02 | C |
| ATOM | 17471 | CD1 | LEU | I | 101 | 19.975 | 62.793 | 55.593 | 1.00 | 45.74 | C |
| ATOM | 17475 | CD2 | LEU | I | 101 | 18.450 | 60.925 | 54.996 | 1.00 | 49.35 | C |
| ATOM | 17479 | C | LEU | I | 101 | 17.082 | 64.732 | 52.529 | 1.00 | 47.95 | C |
| ATOM | 17480 | O | LEU | I | 101 | 16.651 | 65.567 | 53.325 | 1.00 | 53.94 | O |
| ATOM | 17481 | N | VAL | I | 102 | 16.510 | 64.493 | 51.360 | 1.00 | 42.19 | N |
| ATOM | 17483 | CA | VAL | I | 102 | 15.395 | 65.282 | 50.870 | 1.00 | 42.39 | C |
| ATOM | 17485 | CB | VAL | I | 102 | 15.685 | 65.800 | 49.447 | 1.00 | 41.48 | C |
| ATOM | 17487 | CG1 | VAL | I | 102 | 14.558 | 66.662 | 48.968 | 1.00 | 38.99 | C |
| ATOM | 17491 | CG2 | VAL | I | 102 | 17.008 | 66.559 | 49.435 | 1.00 | 47.46 | C |
| ATOM | 17495 | C | VAL | I | 102 | 14.207 | 64.351 | 50.771 | 1.00 | 45.99 | C |
| ATOM | 17496 | O | VAL | I | 102 | 14.301 | 63.279 | 50.157 | 1.00 | 43.66 | O |
| ATOM | 17497 | N | PHE | I | 103 | 13.094 | 64.783 | 51.352 | 1.00 | 46.46 | N |
| ATOM | 17499 | CA | PHE | I | 103 | 11.862 | 64.007 | 51.331 | 1.00 | 45.56 | C |
| ATOM | 17501 | CB | PHE | I | 103 | 11.335 | 63.827 | 52.743 | 1.00 | 45.73 | C |
| ATOM | 17504 | CG | PHE | I | 103 | 12.344 | 63.292 | 53.709 | 1.00 | 46.32 | C |
| ATOM | 17505 | CD1 | PHE | I | 103 | 13.392 | 62.528 | 53.273 | 1.00 | 27.75 | C |
| ATOM | 17507 | CE1 | PHE | I | 103 | 14.287 | 62.019 | 54.195 | 1.00 | 48.47 | C |
| ATOM | 17509 | CZ | PHE | I | 103 | 14.102 | 62.198 | 55.554 | 1.00 | 21.37 | C |
| ATOM | 17511 | CE2 | PHE | I | 103 | 13.091 | 62.990 | 55.989 | 1.00 | 38.88 | C |
| ATOM | 17513 | CD2 | PHE | I | 103 | 12.195 | 63.505 | 55.073 | 1.00 | 45.45 | C |
| ATOM | 17515 | C | PHE | I | 103 | 10.817 | 64.791 | 50.562 | 1.00 | 46.84 | C |
| ATOM | 17516 | O | PHE | I | 103 | 10.364 | 65.847 | 51.044 | 1.00 | 38.93 | O |
| ATOM | 17517 | N | GLU | I | 104 | 10.431 | 64.241 | 49.406 | 1.00 | 48.48 | N |
| ATOM | 17519 | CA | GLU | I | 104 | 9.409 | 64.826 | 48.536 | 1.00 | 49.61 | C |
| ATOM | 17521 | CB | GLU | I | 104 | 9.855 | 64.772 | 47.078 | 1.00 | 53.58 | C |
| ATOM | 17524 | CG | GLU | I | 104 | 10.798 | 65.906 | 46.697 | 1.00 | 60.49 | C |
| ATOM | 17527 | CD | GLU | I | 104 | 11.580 | 65.645 | 45.428 | 1.00 | 60.14 | C |
| ATOM | 17528 | OE1 | GLU | I | 104 | 11.604 | 64.510 | 44.922 | 1.00 | 41.85 | O |
| ATOM | 17529 | OE2 | GLU | I | 104 | 12.184 | 66.599 | 44.914 | 1.00 | 87.16 | O |
| ATOM | 17530 | C | GLU | I | 104 | 8.107 | 64.070 | 48.687 | 1.00 | 48.81 | C |
| ATOM | 17531 | O | GLU | I | 104 | 8.000 | 62.942 | 48.226 | 1.00 | 47.29 | O |
| ATOM | 17532 | N | ALA | I | 105 | 7.168 | 64.664 | 49.422 | 1.00 | 51.66 | N |
| ATOM | 17534 | CA | ALA | I | 105 | 5.798 | 64.170 | 49.550 | 1.00 | 53.99 | C |
| ATOM | 17536 | CB | ALA | I | 105 | 4.944 | 65.149 | 50.318 | 1.00 | 46.11 | C |
| ATOM | 17540 | C | ALA | I | 105 | 5.199 | 64.021 | 48.179 | 1.00 | 60.12 | C |
| ATOM | 17541 | O | ALA | I | 105 | 5.432 | 64.861 | 47.330 | 1.00 | 59.52 | O |
| ATOM | 17542 | N | PRO | I | 106 | 4.335 | 63.031 | 48.006 | 1.00 | 76.19 | N |
| ATOM | 17543 | CA | PRO | I | 106 | 3.799 | 62.698 | 46.687 | 1.00 | 87.83 | C |
| ATOM | 17545 | CB | PRO | I | 106 | 2.574 | 61.838 | 47.033 | 1.00 | 88.70 | C |
| ATOM | 17548 | CG | PRO | I | 106 | 2.985 | 61.126 | 48.287 | 1.00 | 80.95 | C |
| ATOM | 17551 | CD | PRO | I | 106 | 3.750 | 62.176 | 49.053 | 1.00 | 78.03 | C |
| ATOM | 17554 | C | PRO | I | 106 | 3.444 | 63.912 | 45.805 | 1.00 | 92.73 | C |
| ATOM | 17555 | O | PRO | I | 106 | 4.313 | 64.490 | 45.139 | 1.00 | 94.60 | O |
| ATOM | 17556 | N | ASN | I | 107 | 2.170 | 64.262 | 45.707 | 1.00 | 95.37 | N |
| ATOM | 17558 | CA | ASN | I | 107 | 1.845 | 65.456 | 44.933 | 1.00 | 103.14 | C |
| ATOM | 17560 | CB | ASN | I | 107 | 0.344 | 65.525 | 44.534 | 1.00 | 107.19 | C |
| ATOM | 17563 | CG | ASN | I | 107 | 0.067 | 65.030 | 43.096 | 1.00 | 111.32 | C |
| ATOM | 17564 | OD1 | ASN | I | 107 | 0.272 | 65.759 | 42.121 | 1.00 | 116.12 | O |
| ATOM | 17565 | ND2 | ASN | I | 107 | −0.421 | 63.796 | 42.970 | 1.00 | 114.13 | N |
| ATOM | 17568 | C | ASN | I | 107 | 2.270 | 66.602 | 45.850 | 1.00 | 99.87 | C |
| ATOM | 17569 | O | ASN | I | 107 | 3.142 | 67.398 | 45.490 | 1.00 | 83.98 | O |
| ATOM | 17570 | N | GLN | I | 108 | 1.726 | 66.585 | 47.071 | 1.00 | 104.58 | N |
| ATOM | 17572 | CA | GLN | I | 108 | 0.920 | 67.704 | 47.591 | 1.00 | 106.79 | C |
| ATOM | 17574 | CB | GLN | I | 108 | −0.512 | 67.334 | 48.063 | 1.00 | 107.60 | C |
| ATOM | 17577 | CG | GLN | I | 108 | −0.755 | 65.908 | 48.575 | 1.00 | 106.61 | C |
| ATOM | 17580 | CD | GLN | I | 108 | 0.396 | 65.363 | 49.404 | 1.00 | 106.63 | C |
| ATOM | 17581 | OE1 | GLN | I | 108 | 1.049 | 64.398 | 48.999 | 1.00 | 105.00 | O |
| ATOM | 17582 | NE2 | GLN | I | 108 | 0.656 | 65.983 | 50.555 | 1.00 | 106.31 | N |
| ATOM | 17585 | C | GLN | I | 108 | 1.701 | 68.387 | 48.702 | 1.00 | 98.30 | C |
| ATOM | 17586 | O | GLN | I | 108 | 1.991 | 67.775 | 49.731 | 1.00 | 102.06 | O |
| ATOM | 17587 | N | GLU | I | 109 | 2.151 | 69.606 | 48.417 | 1.00 | 86.83 | N |
| ATOM | 17589 | CA | GLU | I | 109 | 3.571 | 69.800 | 48.231 | 1.00 | 74.11 | C |
| ATOM | 17591 | CB | GLU | I | 109 | 3.914 | 70.682 | 47.035 | 1.00 | 79.67 | C |
| ATOM | 17594 | CG | GLU | I | 109 | 5.188 | 70.194 | 46.351 | 1.00 | 90.04 | C |
| ATOM | 17597 | CD | GLU | I | 109 | 4.953 | 69.341 | 45.111 | 1.00 | 98.06 | C |
| ATOM | 17598 | OE1 | GLU | I | 109 | 4.425 | 68.202 | 45.179 | 1.00 | 70.40 | O |
| ATOM | 17599 | OE2 | GLU | I | 109 | 5.353 | 69.820 | 44.032 | 1.00 | 114.55 | O |
| ATOM | 17600 | C | GLU | I | 109 | 4.216 | 70.210 | 49.542 | 1.00 | 60.83 | C |
| ATOM | 17601 | O | GLU | I | 109 | 3.981 | 71.265 | 50.116 | 1.00 | 47.62 | O |
| ATOM | 17602 | N | LYS | I | 110 | 4.905 | 69.218 | 50.071 | 1.00 | 53.64 | N |
| ATOM | 17604 | CA | LYS | I | 110 | 5.699 | 69.314 | 51.268 | 1.00 | 49.94 | C |
| ATOM | 17606 | CB | LYS | I | 110 | 5.186 | 68.313 | 52.282 | 1.00 | 48.09 | C |
| ATOM | 17609 | CG | LYS | I | 110 | 6.032 | 68.171 | 53.531 | 1.00 | 47.62 | C |
| ATOM | 17612 | CD | LYS | I | 110 | 5.168 | 68.088 | 54.810 | 1.00 | 59.44 | C |
| ATOM | 17615 | CE | LYS | I | 110 | 5.575 | 66.974 | 55.771 | 1.00 | 53.39 | C |
| ATOM | 17618 | NZ | LYS | I | 110 | 5.667 | 67.427 | 57.177 | 1.00 | 36.63 | N |
| ATOM | 17622 | C | LYS | I | 110 | 7.041 | 68.829 | 50.790 | 1.00 | 48.49 | C |
| ATOM | 17623 | O | LYS | I | 110 | 7.149 | 67.702 | 50.339 | 1.00 | 44.14 | O |
| ATOM | 17624 | N | VAL | I | 111 | 8.060 | 69.673 | 50.856 | 1.00 | 48.67 | N |
| ATOM | 17626 | CA | VAL | I | 111 | 9.401 | 69.166 | 50.645 | 1.00 | 48.61 | C |
| ATOM | 17628 | CB | VAL | I | 111 | 10.032 | 69.837 | 49.433 | 1.00 | 39.21 | C |
| ATOM | 17630 | CG1 | VAL | I | 111 | 11.344 | 69.196 | 49.122 | 1.00 | 40.48 | C |
| ATOM | 17634 | CG2 | VAL | I | 111 | 9.111 | 69.646 | 48.277 | 1.00 | 41.92 | C |
| ATOM | 17638 | C | VAL | I | 111 | 10.225 | 69.358 | 51.888 | 1.00 | 46.61 | C |
| ATOM | 17639 | O | VAL | I | 111 | 10.172 | 70.427 | 52.491 | 1.00 | 55.46 | O |
| ATOM | 17640 | N | SER | I | 112 | 10.913 | 68.310 | 52.318 | 1.00 | 44.58 | N |
| ATOM | 17642 | CA | SER | I | 112 | 11.788 | 68.440 | 53.485 | 1.00 | 43.23 | C |
| ATOM | 17644 | CB | SER | I | 112 | 11.375 | 67.478 | 54.613 | 1.00 | 39.23 | C |
| ATOM | 17647 | OG | SER | I | 112 | 10.061 | 67.728 | 55.114 | 1.00 | 36.22 | O |
| ATOM | 17649 | C | SER | I | 112 | 13.250 | 68.256 | 53.085 | 1.00 | 41.50 | C |
| ATOM | 17650 | O | SER | I | 112 | 13.625 | 67.244 | 52.490 | 1.00 | 40.16 | O |
| ATOM | 17651 | N | ASP | I | 113 | 14.067 | 69.264 | 53.370 | 1.00 | 49.52 | N |
| ATOM | 17653 | CA | ASP | I | 113 | 15.524 | 69.170 | 53.232 | 1.00 | 48.61 | C |
| ATOM | 17655 | CB | ASP | I | 113 | 16.070 | 70.440 | 52.598 | 1.00 | 46.58 | C |
| ATOM | 17658 | CG | ASP | I | 113 | 16.214 | 70.287 | 51.121 | 1.00 | 70.68 | C |
| ATOM | 17659 | OD1 | ASP | I | 113 | 17.345 | 69.949 | 50.702 | 1.00 | 105.99 | O |
| ATOM | 17660 | OD2 | ASP | I | 113 | 15.254 | 70.409 | 50.319 | 1.00 | 75.57 | O |
| ATOM | 17661 | C | ASP | I | 113 | 16.137 | 69.058 | 54.599 | 1.00 | 44.61 | C |
| ATOM | 17662 | O | ASP | I | 113 | 16.042 | 69.998 | 55.376 | 1.00 | 47.73 | O |
| ATOM | 17663 | N | TYR | I | 114 | 16.727 | 67.913 | 54.909 | 1.00 | 46.30 | N |
| ATOM | 17665 | CA | TYR | I | 114 | 17.497 | 67.765 | 56.147 | 1.00 | 46.56 | C |
| ATOM | 17667 | CB | TYR | I | 114 | 17.085 | 66.504 | 56.919 | 1.00 | 50.05 | C |
| ATOM | 17670 | CG | TYR | I | 114 | 15.744 | 66.646 | 57.569 | 1.00 | 31.38 | C |
| ATOM | 17671 | CD1 | TYR | I | 114 | 14.597 | 66.331 | 56.885 | 1.00 | 37.64 | C |
| ATOM | 17673 | CE1 | TYR | I | 114 | 13.378 | 66.509 | 57.457 | 1.00 | 43.21 | C |
| ATOM | 17675 | CZ | TYR | I | 114 | 13.270 | 66.987 | 58.720 | 1.00 | 25.69 | C |
| ATOM | 17676 | OH | TYR | I | 114 | 12.012 | 67.177 | 59.200 | 1.00 | 46.96 | O |
| ATOM | 17678 | CE2 | TYR | I | 114 | 14.386 | 67.260 | 59.444 | 1.00 | 49.86 | C |
| ATOM | 17680 | CD2 | TYR | I | 114 | 15.623 | 67.099 | 58.850 | 1.00 | 41.44 | C |
| ATOM | 17682 | C | TYR | I | 114 | 18.996 | 67.707 | 55.863 | 1.00 | 45.47 | C |
| ATOM | 17683 | O | TYR | I | 114 | 19.449 | 67.408 | 54.745 | 1.00 | 44.42 | O |
| ATOM | 17684 | N | GLU | I | 115 | 19.783 | 67.954 | 56.899 | 1.00 | 42.57 | N |
| ATOM | 17686 | CA | GLU | I | 115 | 21.225 | 67.807 | 56.739 | 1.00 | 44.61 | C |
| ATOM | 17688 | CB | GLU | I | 115 | 21.809 | 69.160 | 56.374 | 1.00 | 38.79 | C |
| ATOM | 17691 | CG | GLU | I | 115 | 23.303 | 69.287 | 56.532 | 1.00 | 64.17 | C |
| ATOM | 17694 | CD | GLU | I | 115 | 23.872 | 70.277 | 55.520 | 1.00 | 87.16 | C |
| ATOM | 17695 | OE1 | GLU | I | 115 | 23.671 | 69.995 | 54.300 | 1.00 | 79.67 | O |
| ATOM | 17696 | OE2 | GLU | I | 115 | 24.497 | 71.300 | 55.953 | 1.00 | 45.60 | O |
| ATOM | 17697 | C | GLU | I | 115 | 21.833 | 67.290 | 58.015 | 1.00 | 39.67 | C |
| ATOM | 17698 | O | GLU | I | 115 | 21.653 | 67.887 | 59.061 | 1.00 | 40.37 | O |
| ATOM | 17699 | N | MET | I | 116 | 22.518 | 66.161 | 57.933 | 1.00 | 40.54 | N |
| ATOM | 17701 | CA | MET | I | 116 | 23.040 | 65.492 | 59.129 | 1.00 | 43.08 | C |
| ATOM | 17703 | CB | MET | I | 116 | 22.580 | 64.031 | 59.101 | 1.00 | 47.47 | C |
| ATOM | 17706 | CG | MET | I | 116 | 23.376 | 63.027 | 59.915 | 1.00 | 44.96 | C |
| ATOM | 17709 | SD | MET | I | 116 | 22.375 | 61.519 | 59.985 | 1.00 | 50.50 | S |
| ATOM | 17710 | CE | MET | I | 116 | 22.956 | 60.628 | 58.559 | 1.00 | 45.18 | C |
| ATOM | 17714 | C | MET | I | 116 | 24.567 | 65.547 | 59.186 | 1.00 | 39.96 | C |
| ATOM | 17715 | O | MET | I | 116 | 26.267 | 65.339 | 58.199 | 1.00 | 40.35 | O |
| ATOM | 17716 | N | LYS | I | 117 | 25.083 | 65.815 | 60.368 | 1.00 | 43.81 | N |
| ATOM | 17718 | CA | LYS | I | 117 | 26.527 | 65.830 | 60.570 | 1.00 | 46.71 | C |
| ATOM | 17720 | CB | LYS | I | 117 | 26.844 | 66.516 | 61.907 | 1.00 | 42.20 | C |
| ATOM | 17723 | CG | LYS | I | 117 | 26.472 | 68.007 | 61.913 | 1.00 | 56.20 | C |
| ATOM | 17726 | CD | LYS | I | 117 | 26.875 | 68.734 | 63.198 | 1.00 | 63.52 | C |
| ATOM | 17729 | CE | LYS | I | 117 | 26.182 | 70.079 | 63.316 | 1.00 | 57.69 | C |
| ATOM | 17732 | NZ | LYS | I | 117 | 27.078 | 71.218 | 63.622 | 1.00 | 72.08 | N |
| ATOM | 17736 | C | LYS | I | 117 | 27.068 | 64.397 | 60.528 | 1.00 | 45.75 | C |
| ATOM | 17737 | O | LYS | I | 117 | 26.498 | 63.479 | 61.092 | 1.00 | 49.66 | O |
| ATOM | 17738 | N | LEU | I | 118 | 28.174 | 64.196 | 59.836 | 1.00 | 49.65 | N |
| ATOM | 17740 | CA | LEU | I | 118 | 28.866 | 62.920 | 59.913 | 1.00 | 49.60 | C |
| ATOM | 17742 | CB | LEU | I | 118 | 29.583 | 62.652 | 58.592 | 1.00 | 52.15 | C |
| ATOM | 17745 | CG | LEU | I | 118 | 28.538 | 62.689 | 57.475 | 1.00 | 56.05 | C |
| ATOM | 17747 | CD1 | LEU | I | 118 | 29.154 | 62.895 | 56.112 | 1.00 | 49.20 | C |
| ATOM | 17751 | CD2 | LEU | I | 118 | 27.656 | 61.429 | 57.500 | 1.00 | 55.62 | C |
| ATOM | 17755 | C | LEU | I | 118 | 29.856 | 62.892 | 61.049 | 1.00 | 45.67 | C |
| ATOM | 17756 | O | LEU | I | 118 | 30.251 | 63.895 | 61.610 | 1.00 | 49.11 | O |
| ATOM | 17757 | N | MET | I | 119 | 30.286 | 61.712 | 61.421 | 1.00 | 46.92 | N |
| ATOM | 17759 | CA | MET | I | 119 | 31.280 | 61.660 | 62.465 | 1.00 | 44.64 | C |
| ATOM | 17761 | CB | MET | I | 119 | 30.652 | 61.074 | 63.726 | 1.00 | 45.95 | C |
| ATOM | 17764 | CG | MET | I | 119 | 30.098 | 59.672 | 63.511 | 1.00 | 46.41 | C |
| ATOM | 17767 | SD | MET | I | 119 | 29.433 | 58.926 | 65.005 | 1.00 | 81.52 | S |
| ATOM | 17768 | CE | MET | I | 119 | 28.624 | 60.432 | 65.781 | 1.00 | 102.70 | C |
| ATOM | 17772 | C | MET | I | 119 | 32.391 | 60.787 | 61.896 | 1.00 | 52.25 | C |
| ATOM | 17773 | O | MET | I | 119 | 32.406 | 60.421 | 60.700 | 1.00 | 51.00 | O |
| ATOM | 17774 | N | ASP | I | 120 | 33.382 | 60.537 | 62.734 | 1.00 | 58.44 | N |
| ATOM | 17776 | CA | ASP | I | 120 | 34.635 | 59.988 | 62.262 | 1.00 | 61.30 | C |
| ATOM | 17778 | CB | ASP | I | 120 | 35.804 | 60.865 | 62.699 | 1.00 | 68.82 | C |
| ATOM | 17781 | CG | ASP | I | 120 | 36.277 | 61.757 | 61.593 | 1.00 | 91.22 | C |
| ATOM | 17782 | OD1 | ASP | I | 120 | 36.498 | 61.231 | 60.474 | 1.00 | 115.91 | O |
| ATOM | 17783 | OD2 | ASP | I | 120 | 36.388 | 62.990 | 61.745 | 1.00 | 111.98 | O |
| ATOM | 17784 | C | ASP | I | 120 | 34.671 | 58.744 | 63.069 | 1.00 | 56.75 | C |
| ATOM | 17785 | O | ASP | I | 120 | 34.771 | 58.840 | 64.284 | 1.00 | 54.70 | O |
| ATOM | 17786 | N | LEU | I | 121 | 34.513 | 57.595 | 62.436 | 1.00 | 55.05 | N |
| ATOM | 17788 | CA | LEU | I | 121 | 34.894 | 56.384 | 63.130 | 1.00 | 66.68 | C |
| ATOM | 17790 | CB | LEU | I | 121 | 33.688 | 55.520 | 63.462 | 1.00 | 72.24 | C |
| ATOM | 17793 | CG | LEU | I | 121 | 32.316 | 56.017 | 62.994 | 1.00 | 66.60 | C |
| ATOM | 17795 | CD1 | LEU | I | 121 | 31.893 | 55.261 | 61.744 | 1.00 | 76.12 | C |
| ATOM | 17799 | CD2 | LEU | I | 121 | 31.328 | 55.727 | 64.115 | 1.00 | 74.84 | C |
| ATOM | 17803 | C | LEU | I | 121 | 35.844 | 55.622 | 62.258 | 1.00 | 69.60 | C |
| ATOM | 17804 | O | LEU | I | 121 | 35.748 | 55.675 | 61.043 | 1.00 | 81.05 | O |
| ATOM | 17805 | N | ASP | I | 122 | 36.776 | 54.915 | 62.864 | 1.00 | 74.87 | N |
| ATOM | 17807 | CA | ASP | I | 122 | 37.473 | 53.927 | 62.070 | 1.00 | 85.30 | C |
| ATOM | 17809 | CB | ASP | I | 122 | 39.003 | 54.091 | 62.127 | 1.00 | 91.98 | C |
| ATOM | 17812 | CG | ASP | I | 122 | 39.564 | 53.981 | 63.548 | 1.00 | 105.98 | C |
| ATOM | 17813 | OD1 | ASP | I | 122 | 40.768 | 53.649 | 63.673 | 1.00 | 108.89 | O |
| ATOM | 17814 | OD2 | ASP | I | 122 | 38.895 | 54.204 | 64.595 | 1.00 | 121.20 | O |
| ATOM | 17815 | C | ASP | I | 122 | 36.984 | 52.570 | 62.549 | 1.00 | 81.54 | C |
| ATOM | 17816 | O | ASP | I | 122 | 36.694 | 52.356 | 63.744 | 1.00 | 75.10 | O |
| ATOM | 17817 | N | VAL | I | 123 | 36.801 | 51.697 | 61.567 | 1.00 | 74.40 | N |
| ATOM | 17819 | CA | VAL | I | 123 | 36.314 | 50.361 | 61.837 | 1.00 | 73.08 | C |
| ATOM | 17821 | CB | VAL | I | 123 | 34.792 | 50.299 | 61.818 | 1.00 | 76.95 | C |
| ATOM | 17823 | CG1 | VAL | I | 123 | 34.274 | 50.544 | 63.245 | 1.00 | 83.60 | C |
| ATOM | 17827 | CG2 | VAL | I | 123 | 34.214 | 51.292 | 60.761 | 1.00 | 64.61 | C |
| ATOM | 17831 | C | VAL | I | 123 | 36.842 | 49.444 | 60.775 | 1.00 | 67.45 | C |
| ATOM | 17832 | O | VAL | I | 123 | 36.677 | 49.680 | 59.593 | 1.00 | 72.38 | O |
| ATOM | 17833 | N | GLU | I | 124 | 37.514 | 48.398 | 61.194 | 1.00 | 65.75 | N |
| ATOM | 17835 | CA | GLU | I | 124 | 37.954 | 47.422 | 60.225 | 1.00 | 68.53 | C |
| ATOM | 17837 | CB | GLU | I | 124 | 38.928 | 46.446 | 60.881 | 1.00 | 72.89 | C |
| ATOM | 17840 | CG | GLU | I | 124 | 39.557 | 46.993 | 62.147 | 1.00 | 80.77 | C |
| ATOM | 17843 | CD | GLU | I | 124 | 40.590 | 46.039 | 62.685 | 1.00 | 95.82 | C |
| ATOM | 17844 | OE1 | GLU | I | 124 | 41.574 | 45.802 | 61.946 | 1.00 | 94.63 | O |
| ATOM | 17845 | OE2 | GLU | I | 124 | 40.378 | 45.515 | 63.804 | 1.00 | 100.34 | O |
| ATOM | 17846 | C | GLU | I | 124 | 36.739 | 46.676 | 59.703 | 1.00 | 58.63 | C |
| ATOM | 17847 | O | GLU | I | 124 | 35.989 | 46.107 | 60.494 | 1.00 | 67.80 | O |
| ATOM | 17848 | N | GLN | I | 125 | 36.562 | 46.679 | 58.388 | 1.00 | 44.11 | N |
| ATOM | 17850 | CA | GLN | I | 125 | 35.822 | 45.633 | 57.709 | 1.00 | 53.32 | C |
| ATOM | 17852 | CB | GLN | I | 125 | 35.885 | 45.862 | 56.207 | 1.00 | 61.83 | C |
| ATOM | 17855 | CG | GLN | I | 125 | 34.620 | 45.434 | 55.474 | 1.00 | 76.16 | C |
| ATOM | 17858 | CD | GLN | I | 125 | 33.714 | 46.611 | 55.233 | 1.00 | 71.49 | C |
| ATOM | 17859 | OE1 | GLN | I | 125 | 33.112 | 46.721 | 54.174 | 1.00 | 85.03 | O |
| ATOM | 17860 | NE2 | GLN | I | 125 | 33.646 | 47.516 | 56.204 | 1.00 | 87.88 | N |
| ATOM | 17863 | C | GLN | I | 125 | 36.273 | 44.199 | 58.013 | 1.00 | 53.08 | C |
| ATOM | 17864 | O | GLN | I | 125 | 37.452 | 43.876 | 57.983 | 1.00 | 57.13 | O |
| ATOM | 17865 | N | LEU | I | 126 | 35.317 | 43.340 | 58.321 | 1.00 | 50.80 | N |
| ATOM | 17867 | CA | LEU | I | 126 | 35.577 | 41.922 | 58.464 | 1.00 | 56.61 | C |
| ATOM | 17869 | CB | LEU | I | 126 | 34.743 | 41.349 | 59.619 | 1.00 | 54.03 | C |
| ATOM | 17872 | CG | LEU | I | 126 | 35.020 | 42.020 | 60.961 | 1.00 | 48.78 | C |
| ATOM | 17874 | CD1 | LEU | I | 126 | 35.030 | 41.045 | 62.135 | 1.00 | 54.84 | C |
| ATOM | 17878 | CD2 | LEU | I | 126 | 36.341 | 42.683 | 60.893 | 1.00 | 44.25 | C |
| ATOM | 17882 | C | LEU | I | 126 | 35.253 | 41.171 | 57.171 | 1.00 | 59.55 | C |
| ATOM | 17883 | O | LEU | I | 126 | 34.391 | 41.574 | 56.395 | 1.00 | 59.05 | O |
| ATOM | 17884 | N | GLY | I | 127 | 35.934 | 40.054 | 56.951 | 1.00 | 58.93 | N |
| ATOM | 17886 | CA | GLY | I | 127 | 35.823 | 39.384 | 55.675 | 1.00 | 55.76 | C |
| ATOM | 17889 | C | GLY | I | 127 | 34.962 | 38.187 | 55.944 | 1.00 | 60.54 | C |
| ATOM | 17890 | O | GLY | I | 127 | 35.179 | 37.469 | 56.925 | 1.00 | 60.97 | O |
| ATOM | 17891 | N | ILE | I | 128 | 33.922 | 38.044 | 55.134 | 1.00 | 59.92 | N |
| ATOM | 17893 | CA | ILE | I | 128 | 32.901 | 37.071 | 55.439 | 1.00 | 61.72 | C |
| ATOM | 17895 | CB | ILE | I | 128 | 31.523 | 37.707 | 55.522 | 1.00 | 65.40 | C |
| ATOM | 17897 | CG1 | ILE | I | 128 | 31.569 | 38.886 | 56.500 | 1.00 | 65.87 | C |
| ATOM | 17900 | CD1 | ILE | I | 128 | 30.264 | 39.170 | 57.235 | 1.00 | 74.26 | C |
| ATOM | 17904 | CG2 | ILE | I | 128 | 30.522 | 36.643 | 55.926 | 1.00 | 63.70 | C |
| ATOM | 17908 | C | ILE | I | 128 | 32.940 | 36.050 | 54.339 | 1.00 | 60.72 | C |
| ATOM | 17909 | O | ILE | I | 128 | 32.458 | 36.251 | 53.244 | 1.00 | 53.02 | O |
| ATOM | 17910 | N | PRO | I | 129 | 33.572 | 34.937 | 54.640 | 1.00 | 67.47 | N |
| ATOM | 17911 | CA | PRO | I | 129 | 33.829 | 33.931 | 53.613 | 1.00 | 67.31 | C |
| ATOM | 17913 | CB | PRO | I | 129 | 34.500 | 32.792 | 54.381 | 1.00 | 72.83 | C |
| ATOM | 17916 | CG | PRO | I | 129 | 34.310 | 33.096 | 55.834 | 1.00 | 76.19 | C |
| ATOM | 17919 | CD | PRO | I | 129 | 34.069 | 34.574 | 55.977 | 1.00 | 62.04 | C |
| ATOM | 17922 | C | PRO | I | 129 | 32.509 | 33.476 | 53.035 | 1.00 | 72.44 | C |
| ATOM | 17923 | O | PRO | I | 129 | 31.582 | 33.138 | 53.792 | 1.00 | 64.89 | O |
| ATOM | 17924 | N | GLU | I | 130 | 32.447 | 33.509 | 51.703 | 1.00 | 78.45 | N |
| ATOM | 17926 | CA | GLU | I | 130 | 31.788 | 32.484 | 50.879 | 1.00 | 78.76 | C |
| ATOM | 17928 | CB | GLU | I | 130 | 32.566 | 32.253 | 49.574 | 1.00 | 85.10 | C |
| ATOM | 17931 | CG | GLU | I | 130 | 32.266 | 33.245 | 48.455 | 1.00 | 99.84 | C |
| ATOM | 17934 | CD | GLU | I | 130 | 30.898 | 33.054 | 47.807 | 1.00 | 109.15 | C |
| ATOM | 17935 | OE1 | GLU | I | 130 | 29.891 | 32.947 | 48.540 | 1.00 | 107.81 | O |
| ATOM | 17936 | OE2 | GLU | I | 130 | 30.821 | 33.031 | 46.558 | 1.00 | 111.04 | O |
| ATOM | 17937 | C | GLU | I | 130 | 31.645 | 31.157 | 51.608 | 1.00 | 70.78 | C |
| ATOM | 17938 | O | GLU | I | 130 | 32.630 | 30.480 | 51.903 | 1.00 | 68.47 | O |
| ATOM | 17939 | N | GLN | I | 131 | 30.412 | 30.794 | 51.921 | 1.00 | 67.77 | N |
| ATOM | 17941 | CA | GLN | I | 131 | 30.186 | 29.556 | 52.646 | 1.00 | 74.48 | C |
| ATOM | 17943 | CB | GLN | I | 131 | 30.180 | 29.845 | 54.158 | 1.00 | 78.05 | C |
| ATOM | 17946 | CG | GLN | I | 131 | 29.604 | 28.731 | 55.031 | 1.00 | 84.84 | C |
| ATOM | 17949 | CD | GLN | I | 131 | 30.351 | 28.583 | 56.335 | 1.00 | 94.35 | C |
| ATOM | 17950 | OE1 | GLN | I | 131 | 30.549 | 27.467 | 56.817 | 1.00 | 101.44 | O |
| ATOM | 17951 | NE2 | GLN | I | 131 | 30.793 | 29.701 | 56.894 | 1.00 | 97.80 | N |
| ATOM | 17954 | C | GLN | I | 131 | 28.880 | 28.928 | 52.153 | 1.00 | 73.50 | C |
| ATOM | 17955 | O | GLN | I | 131 | 27.933 | 29.643 | 51.810 | 1.00 | 74.71 | O |
| ATOM | 17956 | N | GLU | I | 132 | 28.824 | 27.600 | 52.102 | 1.00 | 70.99 | N |
| ATOM | 17958 | CA | GLU | I | 132 | 27.528 | 26.924 | 52.008 | 1.00 | 70.48 | C |
| ATOM | 17960 | CB | GLU | I | 132 | 27.446 | 25.986 | 50.796 | 1.00 | 75.76 | C |
| ATOM | 17963 | CG | GLU | I | 132 | 28.471 | 24.853 | 50.792 | 1.00 | 86.82 | C |
| ATOM | 17966 | CD | GLU | I | 132 | 28.424 | 24.055 | 49.495 | 1.00 | 108.97 | C |
| ATOM | 17967 | OE1 | GLU | I | 132 | 29.450 | 23.983 | 48.781 | 1.00 | 105.31 | O |
| ATOM | 17968 | OE2 | GLU | I | 132 | 27.342 | 23.514 | 49.171 | 1.00 | 122.88 | O |
| ATOM | 17969 | C | GLU | I | 132 | 27.277 | 26.151 | 53.290 | 1.00 | 64.07 | C |
| ATOM | 17970 | O | GLU | I | 132 | 28.203 | 25.693 | 53.964 | 1.00 | 56.50 | O |
| ATOM | 17971 | N | TYR | I | 133 | 26.007 | 26.072 | 53.656 | 1.00 | 56.19 | N |
| ATOM | 17973 | CA | TYR | I | 133 | 25.666 | 25.849 | 55.044 | 1.00 | 52.19 | C |
| ATOM | 17975 | CB | TYR | I | 133 | 24.744 | 26.962 | 55.580 | 1.00 | 54.89 | C |
| ATOM | 17978 | CG | TYR | I | 133 | 25.418 | 28.306 | 55.826 | 1.00 | 56.23 | C |
| ATOM | 17979 | CD1 | TYR | I | 133 | 25.609 | 29.203 | 54.785 | 1.00 | 45.63 | C |
| ATOM | 17981 | CE1 | TYR | I | 133 | 26.275 | 30.427 | 54.984 | 1.00 | 47.21 | C |
| ATOM | 17983 | CZ | TYR | I | 133 | 26.736 | 30.790 | 56.245 | 1.00 | 57.65 | C |
| ATOM | 17984 | OH | TYR | I | 133 | 27.336 | 32.035 | 56.400 | 1.00 | 42.19 | O |
| ATOM | 17986 | CE2 | TYR | I | 133 | 26.546 | 29.916 | 57.306 | 1.00 | 58.63 | C |
| ATOM | 17988 | CD2 | TYR | I | 133 | 25.887 | 28.673 | 57.093 | 1.00 | 66.89 | C |
| ATOM | 17990 | C | TYR | I | 133 | 25.027 | 24.476 | 55.135 | 1.00 | 47.29 | C |
| ATOM | 17991 | O | TYR | I | 133 | 24.320 | 24.033 | 54.227 | 1.00 | 45.79 | O |
| ATOM | 17992 | N | SER | I | 134 | 25.370 | 23.778 | 56.209 | 1.00 | 48.77 | N |
| ATOM | 17994 | CA | SER | I | 134 | 24.713 | 22.537 | 56.598 | 1.00 | 52.72 | C |
| ATOM | 17996 | CB | SER | I | 134 | 24.976 | 22.217 | 58.072 | 1.00 | 56.35 | C |
| ATOM | 17999 | OG | SER | I | 134 | 26.320 | 21.831 | 58.323 | 1.00 | 57.88 | O |
| ATOM | 18001 | C | SER | I | 134 | 23.214 | 22.552 | 56.376 | 1.00 | 56.28 | C |
| ATOM | 18002 | O | SER | I | 134 | 22.691 | 21.691 | 55.686 | 1.00 | 65.04 | O |
| ATOM | 18003 | N | CYS | I | 135 | 22.502 | 23.489 | 56.991 | 1.00 | 63.20 | N |
| ATOM | 18005 | CA | CYS | I | 135 | 21.034 | 23.471 | 56.953 | 1.00 | 65.38 | C |
| ATOM | 18007 | CB | CYS | I | 135 | 20.448 | 22.722 | 58.190 | 1.00 | 57.73 | C |
| ATOM | 18010 | SG | CYS | I | 135 | 18.810 | 23.309 | 58.688 | 1.00 | 105.49 | S |
| ATOM | 18011 | C | CYS | I | 135 | 20.541 | 24.917 | 56.734 | 1.00 | 52.96 | C |
| ATOM | 18012 | O | CYS | I | 135 | 21.112 | 25.841 | 57.292 | 1.00 | 49.57 | O |
| ATOM | 18013 | N | VAL | I | 136 | 19.585 | 25.114 | 55.825 | 1.00 | 51.62 | N |
| ATOM | 18015 | CA | VAL | I | 136 | 19.058 | 26.440 | 55.454 | 1.00 | 45.09 | C |
| ATOM | 18017 | CB | VAL | I | 136 | 19.495 | 26.894 | 54.038 | 1.00 | 39.67 | C |
| ATOM | 18019 | CG1 | VAL | I | 136 | 18.597 | 28.008 | 53.500 | 1.00 | 33.15 | C |
| ATOM | 18023 | CG2 | VAL | I | 136 | 20.891 | 27.396 | 54.068 | 1.00 | 53.40 | C |
| ATOM | 18027 | C | VAL | I | 136 | 17.535 | 26.496 | 55.453 | 1.00 | 40.71 | C |
| ATOM | 18028 | O | VAL | I | 136 | 16.863 | 25.898 | 54.633 | 1.00 | 41.23 | O |
| ATOM | 18029 | N | VAL | I | 137 | 16.976 | 27.311 | 56.322 | 1.00 | 46.01 | N |
| ATOM | 18031 | CA | VAL | I | 137 | 15.562 | 27.215 | 56.604 | 1.00 | 49.76 | C |
| ATOM | 18033 | CB | VAL | I | 137 | 15.290 | 27.185 | 58.103 | 1.00 | 48.61 | C |
| ATOM | 18035 | CG1 | VAL | I | 137 | 13.852 | 27.510 | 58.318 | 1.00 | 55.73 | C |
| ATOM | 18039 | CG2 | VAL | I | 137 | 15.619 | 25.846 | 58.705 | 1.00 | 55.58 | C |
| ATOM | 18043 | C | VAL | I | 137 | 14.955 | 28.515 | 56.178 | 1.00 | 47.63 | C |
| ATOM | 18044 | O | VAL | I | 137 | 15.333 | 29.558 | 56.692 | 1.00 | 57.08 | O |
| ATOM | 18045 | N | LYS | I | 138 | 13.963 | 28.448 | 55.312 | 1.00 | 49.86 | N |
| ATOM | 18047 | CA | LYS | I | 138 | 13.272 | 29.648 | 54.851 | 1.00 | 47.94 | C |
| ATOM | 18049 | CB | LYS | I | 138 | 13.220 | 29.706 | 53.311 | 1.00 | 47.91 | C |
| ATOM | 18052 | CG | LYS | I | 138 | 12.281 | 30.734 | 52.720 | 1.00 | 55.84 | C |
| ATOM | 18055 | CD | LYS | I | 138 | 12.239 | 30.663 | 51.185 | 1.00 | 73.85 | C |
| ATOM | 18058 | CE | LYS | I | 138 | 12.755 | 31.940 | 50.465 | 1.00 | 75.93 | C |
| ATOM | 18061 | NZ | LYS | I | 138 | 11.818 | 32.338 | 49.349 | 1.00 | 53.20 | N |
| ATOM | 18065 | C | LYS | I | 138 | 11.898 | 29.481 | 55.428 | 1.00 | 44.50 | C |
| ATOM | 18066 | O | LYS | I | 138 | 11.246 | 28.468 | 55.159 | 1.00 | 42.24 | O |
| ATOM | 18067 | N | MET | I | 139 | 11.508 | 30.454 | 56.247 | 1.00 | 42.83 | N |
| ATOM | 18069 | CA | MET | I | 139 | 10.220 | 30.460 | 56.933 | 1.00 | 40.18 | C |
| ATOM | 18071 | CB | MET | I | 139 | 10.407 | 29.991 | 58.358 | 1.00 | 46.03 | C |
| ATOM | 18074 | CG | MET | I | 139 | 11.447 | 30.802 | 59.134 | 1.00 | 46.91 | C |
| ATOM | 18077 | SD | MET | I | 139 | 11.504 | 30.477 | 60.903 | 1.00 | 46.07 | S |
| ATOM | 18078 | CE | MET | I | 139 | 11.207 | 28.786 | 60.917 | 1.00 | 60.35 | C |
| ATOM | 18082 | C | MET | I | 139 | 9.680 | 31.879 | 56.984 | 1.00 | 44.49 | C |
| ATOM | 18083 | O | MET | I | 139 | 10.362 | 32.831 | 56.585 | 1.00 | 51.07 | O |
| ATOM | 18084 | N | PRO | I | 140 | 8.444 | 32.014 | 57.451 | 1.00 | 41.28 | N |
| ATOM | 18085 | CA | PRO | I | 140 | 7.796 | 33.313 | 57.674 | 1.00 | 38.36 | C |
| ATOM | 18087 | CB | PRO | I | 140 | 6.443 | 32.918 | 58.255 | 1.00 | 37.08 | C |
| ATOM | 18090 | CG | PRO | I | 140 | 6.213 | 31.616 | 57.723 | 1.00 | 38.71 | C |
| ATOM | 18093 | CD | PRO | I | 140 | 7.530 | 30.901 | 57.720 | 1.00 | 42.17 | C |
| ATOM | 18096 | C | PRO | I | 140 | 8.488 | 34.155 | 58.715 | 1.00 | 38.79 | C |
| ATOM | 18097 | O | PRO | I | 140 | 8.843 | 33.682 | 59.781 | 1.00 | 44.45 | O |
| ATOM | 18098 | N | SER | I | 141 | 8.616 | 35.437 | 58.451 | 1.00 | 41.24 | N |
| ATOM | 18100 | CA | SER | I | 141 | 9.489 | 36.261 | 59.266 | 1.00 | 38.25 | C |
| ATOM | 18102 | CB | SER | I | 141 | 9.774 | 37.554 | 58.532 | 1.00 | 40.68 | C |
| ATOM | 18105 | OG | SER | I | 141 | 8.537 | 38.160 | 58.221 | 1.00 | 41.30 | O |
| ATOM | 18107 | C | SER | I | 141 | 8.801 | 36.567 | 60.575 | 1.00 | 37.31 | C |
| ATOM | 18108 | O | SER | I | 141 | 9.439 | 36.608 | 61.637 | 1.00 | 44.97 | O |
| ATOM | 18109 | N | GLY | I | 142 | 7.482 | 36.700 | 60.481 | 1.00 | 41.00 | N |
| ATOM | 18111 | CA | GLY | I | 142 | 6.599 | 36.945 | 61.618 | 1.00 | 34.78 | C |
| ATOM | 18114 | C | GLY | I | 142 | 6.625 | 35.812 | 62.598 | 1.00 | 34.63 | C |
| ATOM | 18115 | O | GLY | I | 142 | 6.711 | 36.026 | 63.798 | 1.00 | 38.27 | O |
| ATOM | 18116 | N | GLU | I | 143 | 6.707 | 34.602 | 62.060 | 1.00 | 41.54 | N |
| ATOM | 18118 | CA | GLU | I | 143 | 6.584 | 33.400 | 62.875 | 1.00 | 44.20 | C |
| ATOM | 18120 | CB | GLU | I | 143 | 6.360 | 32.157 | 62.013 | 1.00 | 44.62 | C |
| ATOM | 18123 | CG | GLU | I | 143 | 6.152 | 30.893 | 62.827 | 1.00 | 56.49 | C |
| ATOM | 18126 | CD | GLU | I | 143 | 4.954 | 30.967 | 63.749 | 1.00 | 60.94 | C |
| ATOM | 18127 | OE1 | GLU | I | 143 | 3.839 | 30.888 | 63.214 | 1.00 | 77.03 | O |
| ATOM | 18128 | OE2 | GLU | I | 143 | 5.114 | 31.057 | 64.988 | 1.00 | 64.28 | O |
| ATOM | 18129 | C | GLU | I | 143 | 7.872 | 33.291 | 63.644 | 1.00 | 41.90 | C |
| ATOM | 18130 | O | GLU | I | 143 | 7.889 | 32.826 | 64.781 | 1.00 | 48.36 | O |
| ATOM | 18131 | N | PHE | I | 144 | 8.938 | 33.782 | 63.024 | 1.00 | 44.74 | N |
| ATOM | 18133 | CA | PHE | I | 144 | 10.270 | 33.559 | 63.542 | 1.00 | 43.20 | C |
| ATOM | 18135 | CB | PHE | I | 144 | 11.320 | 33.857 | 62.498 | 1.00 | 34.23 | C |
| ATOM | 18138 | CG | PHE | I | 144 | 12.717 | 33.848 | 63.053 | 1.00 | 39.50 | C |
| ATOM | 18139 | CD1 | PHE | I | 144 | 13.216 | 32.725 | 63.653 | 1.00 | 27.01 | C |
| ATOM | 18141 | CE1 | PHE | I | 144 | 14.519 | 32.666 | 64.047 | 1.00 | 36.60 | C |
| ATOM | 18143 | CZ | PHE | I | 144 | 15.297 | 33.776 | 63.974 | 1.00 | 53.66 | C |
| ATOM | 18145 | CE2 | PHE | I | 144 | 14.806 | 34.927 | 63.417 | 1.00 | 43.52 | C |
| ATOM | 18147 | CD2 | PHE | I | 144 | 13.529 | 34.961 | 62.957 | 1.00 | 45.80 | C |
| ATOM | 18149 | C | PHE | I | 144 | 10.405 | 34.564 | 64.659 | 1.00 | 44.47 | C |
| ATOM | 18150 | O | PHE | I | 144 | 11.070 | 34.330 | 65.673 | 1.00 | 46.89 | O |
| ATOM | 18151 | N | ALA | I | 145 | 9.748 | 35.698 | 64.472 | 1.00 | 39.60 | N |
| ATOM | 18153 | CA | ALA | I | 145 | 9.838 | 36.709 | 65.509 | 1.00 | 34.69 | C |
| ATOM | 18155 | CB | ALA | I | 145 | 9.412 | 38.005 | 64.967 | 1.00 | 27.79 | C |
| ATOM | 18159 | C | ALA | I | 145 | 8.964 | 36.272 | 66.681 | 1.00 | 35.67 | C |
| ATOM | 18160 | O | ALA | I | 145 | 9.406 | 36.285 | 67.817 | 1.00 | 35.49 | O |
| ATOM | 18161 | N | ARG | I | 146 | 7.755 | 35.782 | 66.416 | 1.00 | 40.54 | N |
| ATOM | 18163 | CA | ARG | I | 146 | 6.945 | 35.234 | 67.500 | 1.00 | 38.75 | C |
| ATOM | 18165 | CB | ARG | I | 146 | 5.634 | 34.644 | 66.988 | 1.00 | 41.52 | C |
| ATOM | 18168 | CG | ARG | I | 146 | 4.497 | 34.639 | 68.060 | 1.00 | 64.76 | C |
| ATOM | 18171 | CD | ARG | I | 146 | 3.057 | 34.467 | 67.498 | 1.00 | 94.07 | C |
| ATOM | 18174 | NE | ARG | I | 146 | 2.435 | 35.676 | 66.916 | 1.00 | 100.32 | N |
| ATOM | 18176 | CZ | ARG | I | 146 | 1.960 | 36.726 | 67.606 | 1.00 | 104.33 | C |
| ATOM | 18177 | NH1 | ARG | I | 146 | 1.389 | 37.725 | 66.953 | 1.00 | 98.66 | N |
| ATOM | 18180 | NH2 | ARG | I | 146 | 2.054 | 36.813 | 68.934 | 1.00 | 101.84 | N |
| ATOM | 18183 | C | ARG | I | 146 | 7.712 | 34.197 | 68.303 | 1.00 | 37.35 | C |
| ATOM | 18184 | O | ARG | I | 146 | 7.704 | 34.243 | 69.519 | 1.00 | 29.67 | O |
| ATOM | 18185 | N | ILE | I | 147 | 8.373 | 33.271 | 67.604 | 1.00 | 44.34 | N |
| ATOM | 18187 | CA | ILE | I | 147 | 9.120 | 32.192 | 68.237 | 1.00 | 40.91 | C |
| ATOM | 18189 | CB | ILE | I | 147 | 9.590 | 31.165 | 67.205 | 1.00 | 39.84 | C |
| ATOM | 18191 | CG1 | ILE | I | 147 | 8.405 | 30.285 | 66.787 | 1.00 | 50.36 | C |
| ATOM | 18194 | CD1 | ILE | I | 147 | 8.677 | 29.338 | 65.597 | 1.00 | 26.10 | C |
| ATOM | 18198 | CG2 | ILE | I | 147 | 10.608 | 30.203 | 67.796 | 1.00 | 37.33 | C |
| ATOM | 18202 | C | ILE | I | 147 | 10.282 | 32.670 | 69.089 | 1.00 | 37.72 | C |
| ATOM | 18203 | O | ILE | I | 147 | 10.460 | 32.165 | 70.177 | 1.00 | 37.22 | O |
| ATOM | 18204 | N | CYS | I | 148 | 11.047 | 33.651 | 68.627 | 1.00 | 40.15 | N |
| ATOM | 18206 | CA | CYS | I | 148 | 12.103 | 34.226 | 69.447 | 1.00 | 38.10 | C |
| ATOM | 18208 | CB | CYS | I | 148 | 12.985 | 35.180 | 68.653 | 1.00 | 41.38 | C |
| ATOM | 18211 | SG | CYS | I | 148 | 13.901 | 34.294 | 67.382 | 1.00 | 42.43 | S |
| ATOM | 18212 | C | CYS | I | 148 | 11.559 | 34.948 | 70.644 | 1.00 | 38.47 | C |
| ATOM | 18213 | O | CYS | I | 148 | 12.129 | 34.858 | 71.732 | 1.00 | 54.81 | O |
| ATOM | 18214 | N | ARG | I | 149 | 10.447 | 35.647 | 70.465 | 1.00 | 43.63 | N |
| ATOM | 18216 | CA | ARG | I | 149 | 9.887 | 36.448 | 71.556 | 1.00 | 44.47 | C |
| ATOM | 18218 | CB | ARG | I | 149 | 8.887 | 37.504 | 71.033 | 1.00 | 40.07 | C |
| ATOM | 18221 | CG | ARG | I | 149 | 8.709 | 38.790 | 71.903 | 1.00 | 55.07 | C |
| ATOM | 18224 | CD | ARG | I | 149 | 7.266 | 39.418 | 71.858 | 1.00 | 98.82 | C |
| ATOM | 18227 | NE | ARG | I | 149 | 6.139 | 38.464 | 71.930 | 1.00 | 112.31 | N |
| ATOM | 18229 | CZ | ARG | I | 149 | 5.329 | 38.113 | 70.916 | 1.00 | 105.87 | C |
| ATOM | 18230 | NH1 | ARG | I | 149 | 4.370 | 37.208 | 71.102 | 1.00 | 88.12 | N |
| ATOM | 18233 | NH2 | ARG | I | 149 | 5.478 | 38.638 | 69.704 | 1.00 | 111.77 | N |
| ATOM | 18236 | C | ARG | I | 149 | 9.287 | 35.543 | 72.645 | 1.00 | 38.48 | C |
| ATOM | 18237 | O | ARG | I | 149 | 9.385 | 35.825 | 73.841 | 1.00 | 44.03 | O |
| ATOM | 18238 | N | ASP | I | 150 | 8.749 | 34.400 | 72.256 | 1.00 | 39.08 | N |
| ATOM | 18240 | CA | ASP | I | 150 | 8.093 | 33.566 | 73.241 | 1.00 | 41.26 | C |
| ATOM | 18242 | CB | ASP | I | 150 | 7.121 | 32.631 | 72.575 | 1.00 | 44.36 | C |
| ATOM | 18245 | CG | ASP | I | 150 | 5.904 | 33.327 | 72.037 | 1.00 | 44.58 | C |
| ATOM | 18246 | OD1 | ASP | I | 150 | 5.663 | 34.534 | 72.284 | 1.00 | 59.90 | O |
| ATOM | 18247 | OD2 | ASP | I | 150 | 5.133 | 32.670 | 71.323 | 1.00 | 31.34 | O |
| ATOM | 18248 | C | ASP | I | 150 | 9.106 | 32.722 | 73.966 | 1.00 | 45.55 | C |
| ATOM | 18249 | O | ASP | I | 150 | 9.020 | 32.524 | 75.169 | 1.00 | 47.06 | O |
| ATOM | 18250 | N | LEU | I | 151 | 10.071 | 32.172 | 73.252 | 1.00 | 44.01 | N |
| ATOM | 18252 | CA | LEU | I | 151 | 11.052 | 31.414 | 73.994 | 1.00 | 42.31 | C |
| ATOM | 18254 | CB | LEU | I | 151 | 12.049 | 30.774 | 73.059 | 1.00 | 37.31 | C |
| ATOM | 18257 | CG | LEU | I | 151 | 11.423 | 29.669 | 72.223 | 1.00 | 50.13 | C |
| ATOM | 18259 | CD1 | LEU | I | 151 | 12.528 | 28.892 | 71.515 | 1.00 | 55.31 | C |
| ATOM | 18263 | CD2 | LEU | I | 151 | 10.522 | 28.764 | 73.042 | 1.00 | 39.55 | C |
| ATOM | 18267 | C | LEU | I | 151 | 11.767 | 32.338 | 74.966 | 1.00 | 48.10 | C |
| ATOM | 18268 | O | LEU | I | 151 | 12.275 | 31.853 | 75.981 | 1.00 | 37.04 | O |
| ATOM | 18269 | N | SER | I | 152 | 11.846 | 33.643 | 74.656 | 1.00 | 47.49 | N |
| ATOM | 18271 | CA | SER | I | 152 | 12.661 | 34.537 | 75.487 | 1.00 | 42.90 | C |
| ATOM | 18273 | CB | SER | I | 152 | 12.995 | 35.872 | 74.830 | 1.00 | 37.64 | C |
| ATOM | 18276 | OG | SER | I | 152 | 11.852 | 36.687 | 74.743 | 1.00 | 45.96 | O |
| ATOM | 18278 | C | SER | I | 152 | 12.106 | 34.756 | 76.883 | 1.00 | 34.34 | C |
| ATOM | 18279 | O | SER | I | 152 | 12.890 | 34.934 | 77.794 | 1.00 | 42.75 | O |
| ATOM | 18280 | N | HIS | I | 153 | 10.797 | 34.614 | 77.064 | 1.00 | 36.57 | N |
| ATOM | 18282 | CA | HIS | I | 153 | 10.155 | 34.434 | 78.389 | 1.00 | 42.85 | C |
| ATOM | 18284 | CB | HIS | I | 153 | 8.651 | 34.600 | 78.241 | 1.00 | 37.65 | C |
| ATOM | 18287 | CG | HIS | I | 153 | 8.299 | 35.883 | 77.577 | 1.00 | 62.39 | C |
| ATOM | 18288 | ND1 | HIS | I | 153 | 7.048 | 36.134 | 77.056 | 1.00 | 60.16 | N |
| ATOM | 18290 | CE1 | HIS | I | 153 | 7.048 | 37.354 | 76.545 | 1.00 | 79.45 | C |
| ATOM | 18292 | NE2 | HIS | I | 153 | 8.260 | 37.875 | 76.671 | 1.00 | 53.50 | N |
| ATOM | 18294 | CD2 | HIS | I | 153 | 9.056 | 36.987 | 77.345 | 1.00 | 41.33 | C |
| ATOM | 18296 | C | HIS | I | 153 | 10.356 | 33.132 | 79.157 | 1.00 | 41.26 | C |
| ATOM | 18297 | O | HIS | I | 153 | 10.079 | 33.047 | 80.351 | 1.00 | 45.87 | O |
| ATOM | 18298 | N | ILE | I | 154 | 10.842 | 32.120 | 78.459 | 1.00 | 47.08 | N |
| ATOM | 18300 | CA | ILE | I | 154 | 11.176 | 30.830 | 79.046 | 1.00 | 42.26 | C |
| ATOM | 18302 | CB | ILE | I | 154 | 10.795 | 29.775 | 78.026 | 1.00 | 37.38 | C |
| ATOM | 18304 | CG1 | ILE | I | 154 | 9.320 | 29.935 | 77.672 | 1.00 | 52.56 | C |
| ATOM | 18307 | CD1 | ILE | I | 154 | 8.402 | 29.485 | 78.764 | 1.00 | 47.84 | C |
| ATOM | 18311 | CG2 | ILE | I | 154 | 11.034 | 28.433 | 78.534 | 1.00 | 31.76 | C |
| ATOM | 18315 | C | ILE | I | 154 | 12.660 | 30.734 | 79.415 | 1.00 | 43.00 | C |
| ATOM | 18316 | O | ILE | I | 154 | 13.012 | 30.107 | 80.401 | 1.00 | 48.23 | O |
| ATOM | 18317 | N | GLY | I | 155 | 13.536 | 31.419 | 78.693 | 1.00 | 48.26 | N |
| ATOM | 18319 | CA | GLY | I | 155 | 14.935 | 31.040 | 78.711 | 1.00 | 49.60 | C |
| ATOM | 18322 | C | GLY | I | 155 | 15.825 | 32.009 | 77.971 | 1.00 | 45.75 | C |
| ATOM | 18323 | O | GLY | I | 155 | 15.362 | 32.837 | 77.206 | 1.00 | 40.72 | O |
| ATOM | 18324 | N | ASP | I | 156 | 17.124 | 31.881 | 78.178 | 1.00 | 44.53 | N |
| ATOM | 18326 | CA | ASP | I | 156 | 18.045 | 32.906 | 77.719 | 1.00 | 45.78 | C |
| ATOM | 18328 | CB | ASP | I | 156 | 19.201 | 33.065 | 78.709 | 1.00 | 41.50 | C |
| ATOM | 18331 | CG | ASP | I | 156 | 18.898 | 34.092 | 79.797 | 1.00 | 61.53 | C |
| ATOM | 18332 | OD1 | ASP | I | 156 | 17.788 | 34.678 | 79.789 | 1.00 | 69.53 | O |
| ATOM | 18333 | OD2 | ASP | I | 156 | 19.722 | 34.402 | 80.692 | 1.00 | 76.38 | O |
| ATOM | 18334 | C | ASP | I | 156 | 18.595 | 32.548 | 76.361 | 1.00 | 44.28 | C |
| ATOM | 18335 | O | ASP | I | 156 | 19.135 | 33.408 | 75.686 | 1.00 | 53.26 | O |
| ATOM | 18336 | N | ALA | I | 157 | 18.560 | 31.259 | 76.028 | 1.00 | 46.27 | N |
| ATOM | 18338 | CA | ALA | I | 157 | 19.214 | 30.717 | 74.836 | 1.00 | 40.14 | C |
| ATOM | 18340 | CB | ALA | I | 157 | 20.481 | 29.990 | 75.206 | 1.00 | 31.30 | C |
| ATOM | 18344 | C | ALA | I | 157 | 18.283 | 29.761 | 74.110 | 1.00 | 42.79 | C |
| ATOM | 18345 | O | ALA | I | 157 | 17.485 | 29.049 | 74.746 | 1.00 | 51.98 | O |
| ATOM | 18346 | N | VAL | I | 158 | 18.386 | 29.756 | 72.786 | 1.00 | 33.99 | N |
| ATOM | 18348 | CA | VAL | I | 158 | 17.517 | 28.895 | 72.018 | 1.00 | 40.85 | C |
| ATOM | 18350 | CB | VAL | I | 158 | 16.685 | 29.632 | 70.945 | 1.00 | 39.73 | C |
| ATOM | 18352 | CG1 | VAL | I | 158 | 17.537 | 30.533 | 70.058 | 1.00 | 44.04 | C |
| ATOM | 18356 | CG2 | VAL | I | 158 | 16.016 | 28.590 | 70.092 | 1.00 | 36.04 | C |
| ATOM | 18360 | C | VAL | I | 158 | 18.346 | 27.875 | 71.295 | 1.00 | 39.08 | C |
| ATOM | 18361 | O | VAL | I | 158 | 19.338 | 28.223 | 70.675 | 1.00 | 41.51 | O |
| ATOM | 18362 | N | VAL | I | 159 | 17.890 | 26.631 | 71.310 | 1.00 | 40.30 | N |
| ATOM | 18364 | CA | VAL | I | 159 | 18.589 | 25.575 | 70.596 | 1.00 | 38.96 | C |
| ATOM | 18366 | CB | VAL | I | 159 | 18.662 | 24.263 | 71.405 | 1.00 | 43.42 | C |
| ATOM | 18368 | CG1 | VAL | I | 159 | 19.326 | 23.194 | 70.574 | 1.00 | 47.90 | C |
| ATOM | 18372 | CG2 | VAL | I | 159 | 19.413 | 24.421 | 72.724 | 1.00 | 37.12 | C |
| ATOM | 18376 | C | VAL | I | 159 | 17.773 | 25.311 | 69.359 | 1.00 | 41.78 | C |
| ATOM | 18377 | O | VAL | I | 159 | 16.578 | 25.059 | 69.457 | 1.00 | 49.30 | O |
| ATOM | 18378 | N | ILE | I | 160 | 18.415 | 25.436 | 68.203 | 1.00 | 49.30 | N |
| ATOM | 18380 | CA | ILE | I | 160 | 17.766 | 25.279 | 66.904 | 1.00 | 48.45 | C |
| ATOM | 18382 | CB | ILE | I | 160 | 18.159 | 26.400 | 65.935 | 1.00 | 42.91 | C |
| ATOM | 18384 | CG1 | ILE | I | 160 | 17.832 | 27.755 | 66.536 | 1.00 | 42.62 | C |
| ATOM | 18387 | CD1 | ILE | I | 160 | 17.892 | 28.832 | 65.522 | 1.00 | 35.93 | C |
| ATOM | 18391 | CG2 | ILE | I | 160 | 17.332 | 26.325 | 64.656 | 1.00 | 46.87 | C |
| ATOM | 18395 | C | ILE | I | 160 | 18.261 | 23.968 | 66.329 | 1.00 | 56.15 | C |
| ATOM | 18396 | O | ILE | I | 160 | 19.434 | 23.820 | 66.011 | 1.00 | 56.55 | O |
| ATOM | 18397 | N | SER | I | 161 | 17.346 | 23.019 | 66.237 | 1.00 | 59.13 | N |
| ATOM | 18399 | CA | SER | I | 161 | 17.653 | 21.648 | 65.916 | 1.00 | 58.70 | C |
| ATOM | 18401 | CB | SER | I | 161 | 17.047 | 20.741 | 66.990 | 1.00 | 60.89 | C |
| ATOM | 18404 | OG | SER | I | 161 | 17.451 | 19.388 | 66.844 | 1.00 | 78.17 | O |
| ATOM | 18406 | C | SER | I | 161 | 16.964 | 21.447 | 64.591 | 1.00 | 58.44 | C |
| ATOM | 18407 | O | SER | I | 161 | 15.746 | 21.380 | 64.507 | 1.00 | 56.09 | O |
| ATOM | 18408 | N | CYS | I | 162 | 17.735 | 21.446 | 63.524 | 1.00 | 67.82 | N |
| ATOM | 18410 | CA | CYS | I | 162 | 17.115 | 21.316 | 62.223 | 1.00 | 76.31 | C |
| ATOM | 18412 | CB | CYS | I | 162 | 17.484 | 22.525 | 61.385 | 1.00 | 75.59 | C |
| ATOM | 18415 | SG | CYS | I | 162 | 17.322 | 22.244 | 59.630 | 1.00 | 85.34 | S |
| ATOM | 18416 | C | CYS | I | 162 | 17.441 | 19.987 | 61.519 | 1.00 | 77.42 | C |
| ATOM | 18417 | O | CYS | I | 162 | 18.452 | 19.345 | 61.810 | 1.00 | 72.33 | O |
| ATOM | 18418 | N | ALA | I | 163 | 16.523 | 19.557 | 60.653 | 1.00 | 76.78 | N |
| ATOM | 18420 | CA | ALA | I | 163 | 16.422 | 18.171 | 60.187 | 1.00 | 81.54 | C |
| ATOM | 18422 | CB | ALA | I | 163 | 15.928 | 17.258 | 61.311 | 1.00 | 77.89 | C |
| ATOM | 18426 | C | ALA | I | 163 | 15.440 | 18.148 | 59.002 | 1.00 | 87.14 | C |
| ATOM | 18427 | O | ALA | I | 163 | 14.707 | 19.104 | 58.789 | 1.00 | 94.95 | O |
| ATOM | 18428 | N | LYS | I | 164 | 15.429 | 17.099 | 58.193 | 1.00 | 89.33 | N |
| ATOM | 18430 | CA | LYS | I | 164 | 14.739 | 17.197 | 56.911 | 1.00 | 90.02 | C |
| ATOM | 18432 | CB | LYS | I | 164 | 15.059 | 15.968 | 56.042 | 1.00 | 92.04 | C |
| ATOM | 18435 | CG | LYS | I | 164 | 15.071 | 16.222 | 54.528 | 1.00 | 100.68 | C |
| ATOM | 18438 | CD | LYS | I | 164 | 16.484 | 16.444 | 53.922 | 1.00 | 110.40 | C |
| ATOM | 18441 | CE | LYS | I | 164 | 16.437 | 16.915 | 52.446 | 1.00 | 101.37 | C |
| ATOM | 18444 | NZ | LYS | I | 164 | 17.732 | 17.406 | 51.901 | 1.00 | 74.51 | N |
| ATOM | 18448 | C | LYS | I | 164 | 13.235 | 17.317 | 57.165 | 1.00 | 86.98 | C |
| ATOM | 18449 | O | LYS | I | 164 | 12.489 | 17.961 | 56.425 | 1.00 | 80.11 | O |
| ATOM | 18450 | N | ASP | I | 165 | 12.779 | 16.674 | 58.226 | 1.00 | 86.22 | N |
| ATOM | 18452 | CA | ASP | I | 165 | 11.349 | 16.611 | 58.478 | 1.00 | 91.13 | C |
| ATOM | 18454 | CB | ASP | I | 165 | 11.061 | 15.430 | 59.426 | 1.00 | 92.11 | C |
| ATOM | 18457 | CG | ASP | I | 165 | 11.618 | 15.643 | 60.838 | 1.00 | 96.19 | C |
| ATOM | 18458 | OD1 | ASP | I | 165 | 10.849 | 16.145 | 61.691 | 1.00 | 72.87 | O |
| ATOM | 18459 | OD2 | ASP | I | 165 | 12.798 | 15.358 | 61.178 | 1.00 | 102.65 | O |
| ATOM | 18460 | C | ASP | I | 165 | 10.801 | 17.968 | 59.007 | 1.00 | 93.81 | C |
| ATOM | 18461 | O | ASP | I | 165 | 9.579 | 18.178 | 59.087 | 1.00 | 97.55 | O |
| ATOM | 18462 | N | GLY | I | 166 | 11.697 | 18.895 | 59.357 | 1.00 | 86.70 | N |
| ATOM | 18464 | CA | GLY | I | 166 | 11.316 | 20.103 | 60.072 | 1.00 | 79.75 | C |
| ATOM | 18467 | C | GLY | I | 166 | 12.382 | 20.713 | 60.972 | 1.00 | 74.13 | C |
| ATOM | 18468 | O | GLY | I | 166 | 13.373 | 20.071 | 61.308 | 1.00 | 76.39 | O |
| ATOM | 18469 | N | VAL | I | 167 | 12.137 | 21.945 | 61.415 | 1.00 | 67.37 | N |
| ATOM | 18471 | CA | VAL | I | 167 | 13.012 | 22.637 | 62.358 | 1.00 | 59.77 | C |
| ATOM | 18473 | CB | VAL | I | 167 | 13.445 | 24.005 | 61.802 | 1.00 | 53.96 | C |
| ATOM | 18475 | CG1 | VAL | I | 167 | 12.244 | 24.903 | 61.553 | 1.00 | 44.08 | C |
| ATOM | 18479 | CG2 | VAL | I | 167 | 14.355 | 24.688 | 62.758 | 1.00 | 49.20 | C |
| ATOM | 18483 | C | VAL | I | 167 | 12.377 | 22.825 | 63.751 | 1.00 | 58.91 | C |
| ATOM | 18484 | O | VAL | I | 167 | 11.151 | 22.768 | 63.931 | 1.00 | 57.44 | O |
| ATOM | 18485 | N | LYS | I | 168 | 13.244 | 23.004 | 64.744 | 1.00 | 54.16 | N |
| ATOM | 18487 | CA | LYS | I | 168 | 12.869 | 22.919 | 66.148 | 1.00 | 53.20 | C |
| ATOM | 18489 | CB | LYS | I | 168 | 13.282 | 21.558 | 66.710 | 1.00 | 50.56 | C |
| ATOM | 18492 | CG | LYS | I | 168 | 12.971 | 21.385 | 68.205 | 1.00 | 67.46 | C |
| ATOM | 18495 | CD | LYS | I | 168 | 12.648 | 19.924 | 68.625 | 1.00 | 67.27 | C |
| ATOM | 18498 | CE | LYS | I | 168 | 13.854 | 19.152 | 69.199 | 1.00 | 64.04 | C |
| ATOM | 18501 | NZ | LYS | I | 168 | 13.746 | 18.784 | 70.648 | 1.00 | 68.88 | N |
| ATOM | 18505 | C | LYS | I | 168 | 13.558 | 24.036 | 66.944 | 1.00 | 49.04 | C |
| ATOM | 18506 | O | LYS | I | 188 | 14.711 | 24.357 | 66.696 | 1.00 | 53.93 | O |
| ATOM | 18507 | N | PHE | I | 169 | 12.846 | 24.621 | 67.900 | 1.00 | 42.17 | N |
| ATOM | 18509 | CA | PHE | I | 169 | 13.339 | 25.729 | 68.699 | 1.00 | 42.32 | C |
| ATOM | 18511 | CB | PHE | I | 169 | 12.613 | 27.026 | 68.333 | 1.00 | 40.23 | C |
| ATOM | 18514 | CG | PHE | I | 169 | 12.820 | 27.462 | 66.928 | 1.00 | 42.05 | C |
| ATOM | 18515 | CD1 | PHE | I | 169 | 11.956 | 27.074 | 65.917 | 1.00 | 48.38 | C |
| ATOM | 18517 | CE1 | PHE | I | 169 | 12.149 | 27.525 | 64.607 | 1.00 | 37.73 | C |
| ATOM | 18519 | CZ | PHE | I | 169 | 13.217 | 28.336 | 64.329 | 1.00 | 50.70 | C |
| ATOM | 18521 | CE2 | PHE | I | 169 | 14.081 | 28.718 | 65.344 | 1.00 | 42.54 | C |
| ATOM | 18523 | CD2 | PHE | I | 169 | 13.864 | 28.306 | 66.627 | 1.00 | 41.90 | C |
| ATOM | 18525 | C | PHE | I | 169 | 13.109 | 25.438 | 70.190 | 1.00 | 44.26 | C |
| ATOM | 18526 | O | PHE | I | 169 | 11.970 | 25.384 | 70.658 | 1.00 | 46.47 | O |
| ATOM | 18527 | N | SER | I | 170 | 14.178 | 25.305 | 70.967 | 1.00 | 42.26 | N |
| ATOM | 18529 | CA | SER | I | 170 | 14.011 | 24.913 | 72.351 | 1.00 | 38.92 | C |
| ATOM | 18531 | CB | SER | I | 170 | 14.679 | 23.566 | 72.572 | 1.00 | 38.55 | C |
| ATOM | 18534 | OG | SER | I | 170 | 14.205 | 22.632 | 71.615 | 1.00 | 52.28 | O |
| ATOM | 18536 | C | SER | I | 170 | 14.634 | 25.924 | 73.262 | 1.00 | 37.90 | C |
| ATOM | 18537 | O | SER | I | 170 | 15.708 | 26.476 | 73.012 | 1.00 | 34.03 | O |
| ATOM | 18538 | N | ALA | I | 171 | 13.980 | 26.122 | 74.382 | 1.00 | 37.84 | N |
| ATOM | 18540 | CA | ALA | I | 171 | 14.636 | 26.878 | 75.435 | 1.00 | 41.98 | C |
| ATOM | 18542 | CB | ALA | I | 171 | 14.201 | 28.342 | 75.461 | 1.00 | 40.20 | C |
| ATOM | 18546 | C | ALA | I | 171 | 14.376 | 26.244 | 76.781 | 1.00 | 42.71 | C |
| ATOM | 18547 | O | ALA | I | 171 | 13.581 | 25.327 | 76.952 | 1.00 | 49.58 | O |
| ATOM | 18548 | N | SER | I | 172 | 15.088 | 26.756 | 77.761 | 1.00 | 45.86 | N |
| ATOM | 18550 | CA | SER | I | 172 | 15.013 | 26.185 | 79.077 | 1.00 | 39.55 | C |
| ATOM | 18552 | CB | SER | I | 172 | 15.853 | 24.935 | 79.097 | 1.00 | 24.44 | C |
| ATOM | 18555 | OG | SER | I | 172 | 16.048 | 24.632 | 80.445 | 1.00 | 59.02 | O |
| ATOM | 18557 | C | SER | I | 172 | 15.533 | 27.208 | 80.077 | 1.00 | 38.40 | C |
| ATOM | 18558 | O | SER | I | 172 | 16.537 | 27.899 | 79.831 | 1.00 | 41.90 | O |
| ATOM | 18559 | N | GLY | I | 173 | 14.812 | 27.346 | 81.184 | 1.00 | 36.45 | N |
| ATOM | 18561 | CA | GLY | I | 173 | 15.119 | 28.387 | 82.155 | 1.00 | 38.84 | C |
| ATOM | 18564 | C | GLY | I | 173 | 14.751 | 27.964 | 83.563 | 1.00 | 40.91 | C |
| ATOM | 18565 | O | GLY | I | 173 | 14.391 | 26.821 | 83.801 | 1.00 | 31.36 | O |
| ATOM | 18566 | N | GLU | I | 174 | 14.821 | 28.894 | 84.504 | 1.00 | 50.16 | N |
| ATOM | 18568 | CA | GLU | I | 174 | 14.320 | 28.659 | 85.858 | 1.00 | 55.62 | C |
| ATOM | 18570 | CB | GLU | I | 174 | 14.063 | 30.026 | 86.509 | 1.00 | 71.31 | C |
| ATOM | 18573 | CG | GLU | I | 174 | 13.576 | 29.966 | 87.945 | 1.00 | 88.13 | C |
| ATOM | 18576 | CD | GLU | I | 174 | 14.552 | 29.186 | 88.797 | 1.00 | 101.24 | C |
| ATOM | 18577 | OE1 | GLU | I | 174 | 14.210 | 28.053 | 89.202 | 1.00 | 110.18 | O |
| ATOM | 18578 | OE2 | GLU | I | 174 | 15.679 | 29.690 | 89.000 | 1.00 | 104.09 | O |
| ATOM | 18579 | C | GLU | I | 174 | 13.017 | 27.862 | 85.850 | 1.00 | 51.73 | C |
| ATOM | 18580 | O | GLU | I | 174 | 12.916 | 26.847 | 86.499 | 1.00 | 45.83 | O |
| ATOM | 18581 | N | LEU | I | 175 | 12.010 | 28.335 | 85.124 | 1.00 | 51.63 | N |
| ATOM | 18583 | CA | LEU | I | 175 | 10.645 | 27.908 | 85.348 | 1.00 | 47.09 | C |
| ATOM | 18585 | CB | LEU | I | 175 | 9.661 | 29.029 | 84.958 | 1.00 | 46.62 | C |
| ATOM | 18588 | CG | LEU | I | 175 | 9.560 | 29.458 | 83.495 | 1.00 | 43.25 | C |
| ATOM | 18590 | CD1 | LEU | I | 175 | 8.717 | 28.497 | 82.678 | 1.00 | 40.65 | C |
| ATOM | 18594 | CD2 | LEU | I | 175 | 9.057 | 30.872 | 83.355 | 1.00 | 56.68 | C |
| ATOM | 18598 | C | LEU | I | 175 | 10.331 | 26.590 | 84.636 | 1.00 | 47.27 | C |
| ATOM | 18599 | O | LEU | I | 175 | 9.287 | 25.979 | 84.843 | 1.00 | 50.26 | O |
| ATOM | 18600 | N | GLY | I | 176 | 11.253 | 26.097 | 83.830 | 1.00 | 44.76 | N |
| ATOM | 18602 | CA | GLY | I | 176 | 10.946 | 24.928 | 83.037 | 1.00 | 41.91 | C |
| ATOM | 18605 | C | GLY | I | 176 | 11.429 | 25.156 | 81.623 | 1.00 | 44.20 | C |
| ATOM | 18606 | O | GLY | I | 176 | 12.312 | 25.976 | 81.363 | 1.00 | 50.95 | O |
| ATOM | 18607 | N | ASN | I | 177 | 10.925 | 24.380 | 80.685 | 1.00 | 42.63 | N |
| ATOM | 18609 | CA | ASN | I | 177 | 11.495 | 24.447 | 79.355 | 1.00 | 45.42 | C |
| ATOM | 18611 | CB | ASN | I | 177 | 12.548 | 23.374 | 79.168 | 1.00 | 52.15 | C |
| ATOM | 18614 | CG | ASN | I | 177 | 11.947 | 21.975 | 79.181 | 1.00 | 61.47 | C |
| ATOM | 18615 | OD1 | ASN | I | 177 | 11.114 | 21.686 | 78.343 | 1.00 | 72.18 | O |
| ATOM | 18616 | ND2 | ASN | I | 177 | 12.332 | 21.129 | 80.142 | 1.00 | 60.48 | N |
| ATOM | 18619 | C | ASN | I | 177 | 10.402 | 24.242 | 78.348 | 1.00 | 51.48 | C |
| ATOM | 18620 | O | ASN | I | 177 | 9.279 | 23.874 | 78.688 | 1.00 | 53.43 | O |
| ATOM | 18621 | N | GLY | I | 178 | 10.753 | 24.515 | 77.100 | 1.00 | 57.49 | N |
| ATOM | 18623 | CA | GLY | I | 178 | 9.789 | 24.568 | 76.025 | 1.00 | 54.77 | C |
| ATOM | 18626 | C | GLY | I | 178 | 10.364 | 24.253 | 74.656 | 1.00 | 53.29 | C |
| ATOM | 18627 | O | GLY | I | 178 | 11.576 | 24.319 | 74.388 | 1.00 | 54.49 | O |
| ATOM | 18628 | N | ASN | I | 179 | 9.434 | 24.064 | 73.733 | 1.00 | 48.15 | N |
| ATOM | 18630 | CA | ASN | I | 179 | 9.750 | 23.361 | 72.533 | 1.00 | 45.63 | C |
| ATOM | 18632 | CB | ASN | I | 179 | 9.838 | 21.904 | 72.927 | 1.00 | 52.61 | C |
| ATOM | 18635 | CG | ASN | I | 179 | 11.080 | 21.282 | 72.432 | 1.00 | 54.64 | C |
| ATOM | 18636 | OD1 | ASN | I | 179 | 12.079 | 21.212 | 73.136 | 1.00 | 79.12 | O |
| ATOM | 18637 | ND2 | ASN | I | 179 | 11.051 | 20.861 | 71.176 | 1.00 | 86.88 | N |
| ATOM | 18640 | C | ASN | I | 179 | 8.736 | 23.626 | 71.422 | 1.00 | 44.81 | C |
| ATOM | 18641 | O | ASN | I | 179 | 7.550 | 23.327 | 71.537 | 1.00 | 43.97 | O |
| ATOM | 18642 | N | ILE | I | 180 | 9.201 | 24.273 | 70.361 | 1.00 | 43.05 | N |
| ATOM | 18644 | CA | ILE | I | 180 | 8.329 | 24.646 | 69.268 | 1.00 | 40.33 | C |
| ATOM | 18646 | CB | ILE | I | 180 | 8.296 | 26.177 | 69.093 | 1.00 | 40.73 | C |
| ATOM | 18648 | CG1 | ILE | I | 180 | 7.803 | 26.876 | 70.358 | 1.00 | 42.18 | C |
| ATOM | 18651 | CD1 | ILE | I | 180 | 8.079 | 28.357 | 70.353 | 1.00 | 57.77 | C |
| ATOM | 18655 | CG2 | ILE | I | 180 | 7.317 | 26.545 | 67.962 | 1.00 | 30.01 | C |
| ATOM | 18659 | C | ILE | I | 180 | 8.808 | 23.979 | 67.987 | 1.00 | 41.02 | C |
| ATOM | 18860 | O | ILE | I | 180 | 9.950 | 24.142 | 67.588 | 1.00 | 40.84 | O |
| ATOM | 18661 | N | LYS | I | 181 | 7.968 | 23.184 | 67.337 | 1.00 | 46.93 | N |
| ATOM | 18663 | CA | LYS | I | 181 | 8.476 | 22.494 | 66.153 | 1.00 | 56.99 | C |
| ATOM | 18665 | CB | LYS | I | 181 | 8.738 | 20.990 | 66.359 | 1.00 | 61.08 | C |
| ATOM | 18668 | CG | LYS | I | 181 | 7.879 | 20.221 | 67.388 | 1.00 | 68.94 | C |
| ATOM | 18671 | CD | LYS | I | 181 | 8.588 | 18.942 | 67.930 | 1.00 | 70.80 | C |
| ATOM | 18674 | CE | LYS | I | 181 | 7.677 | 17.710 | 68.053 | 1.00 | 84.29 | C |
| ATOM | 18677 | NZ | LYS | I | 181 | 7.952 | 16.687 | 66.972 | 1.00 | 97.70 | N |
| ATOM | 18681 | C | LYS | I | 181 | 7.628 | 22.766 | 64.923 | 1.00 | 55.90 | C |
| ATOM | 18682 | O | LYS | I | 181 | 6.406 | 22.693 | 64.982 | 1.00 | 61.44 | O |
| ATOM | 18683 | N | LEU | I | 182 | 8.303 | 23.177 | 63.852 | 1.00 | 56.33 | N |
| ATOM | 18685 | CA | LEU | I | 182 | 7.682 | 23.562 | 62.594 | 1.00 | 58.88 | C |
| ATOM | 18687 | CB | LEU | I | 182 | 8.215 | 24.931 | 62.151 | 1.00 | 59.51 | C |
| ATOM | 18690 | CG | LEU | I | 182 | 7.633 | 26.113 | 62.928 | 1.00 | 61.52 | C |
| ATOM | 18692 | CD1 | LEU | I | 182 | 8.571 | 27.271 | 62.843 | 1.00 | 46.11 | C |
| ATOM | 18696 | CD2 | LEU | I | 182 | 6.240 | 26.514 | 62.420 | 1.00 | 64.03 | C |
| ATOM | 18700 | C | LEU | I | 182 | 8.054 | 22.507 | 61.558 | 1.00 | 65.84 | C |
| ATOM | 18701 | O | LEU | I | 182 | 9.237 | 22.267 | 61.295 | 1.00 | 73.22 | O |
| ATOM | 18702 | N | SER | I | 183 | 7.051 | 21.813 | 61.033 | 1.00 | 66.55 | N |
| ATOM | 18704 | CA | SER | I | 183 | 7.278 | 20.806 | 60.006 | 1.00 | 66.50 | C |
| ATOM | 18706 | CB | SER | I | 183 | 6.054 | 19.906 | 59.963 | 1.00 | 61.26 | C |
| ATOM | 18709 | OG | SER | I | 183 | 4.918 | 20.736 | 59.866 | 1.00 | 66.65 | O |
| ATOM | 18711 | C | SER | I | 183 | 7.432 | 21.548 | 58.681 | 1.00 | 64.82 | C |
| ATOM | 18712 | O | SER | I | 183 | 6.865 | 22.631 | 58.551 | 1.00 | 59.70 | O |
| ATOM | 18713 | N | GLN | I | 184 | 8.181 | 20.998 | 57.720 | 1.00 | 66.90 | N |
| ATOM | 18715 | CA | GLN | I | 184 | 8.098 | 21.461 | 56.325 | 1.00 | 69.68 | C |
| ATOM | 18717 | CB | GLN | I | 184 | 8.874 | 20.531 | 55.405 | 1.00 | 66.65 | C |
| ATOM | 18720 | CG | GLN | I | 184 | 10.325 | 20.881 | 55.213 | 1.00 | 73.33 | C |
| ATOM | 18723 | CD | GLN | I | 184 | 10.706 | 20.965 | 53.735 | 1.00 | 73.94 | C |
| ATOM | 18724 | OE1 | GLN | I | 184 | 9.977 | 21.541 | 52.934 | 1.00 | 89.17 | O |
| ATOM | 18725 | NE2 | GLN | I | 184 | 11.857 | 20.427 | 53.386 | 1.00 | 63.18 | 5 |
| ATOM | 18728 | C | GLN | I | 184 | 6.652 | 21.456 | 55.843 | 1.00 | 75.60 | C |
| ATOM | 18729 | O | GLN | I | 184 | 5.945 | 20.475 | 56.038 | 1.00 | 79.73 | O |
| ATOM | 18730 | N | THR | I | 185 | 6.181 | 22.519 | 55.208 | 1.00 | 83.72 | N |
| ATOM | 18732 | CA | THR | I | 185 | 4.826 | 22.479 | 54.673 | 1.00 | 92.45 | C |
| ATOM | 18734 | CB | THR | I | 185 | 4.169 | 23.840 | 54.774 | 1.00 | 89.05 | C |
| ATOM | 18736 | OG1 | THR | I | 185 | 5.171 | 24.848 | 54.628 | 1.00 | 83.49 | O |
| ATOM | 18738 | CG2 | THR | I | 185 | 3.554 | 24.050 | 56.133 | 1.00 | 88.95 | C |
| ATOM | 18742 | C | THR | I | 185 | 4.909 | 22.116 | 53.203 | 1.00 | 100.43 | C |
| ATOM | 18743 | O | THR | I | 185 | 4.000 | 21.488 | 52.635 | 1.00 | 108.73 | O |
| ATOM | 18744 | N | SER | I | 186 | 6.030 | 22.526 | 52.612 | 1.00 | 123.59 | N |
| ATOM | 18746 | CA | SER | I | 186 | 6.035 | 23.232 | 51.329 | 1.00 | 136.42 | C |
| ATOM | 18748 | CB | SER | I | 186 | 6.851 | 22.444 | 50.291 | 1.00 | 138.28 | C |
| ATOM | 18751 | OG | SER | I | 186 | 8.209 | 22.321 | 50.698 | 1.00 | 144.27 | O |
| ATOM | 18753 | C | SER | I | 186 | 4.629 | 23.505 | 50.792 | 1.00 | 142.23 | C |
| ATOM | 18754 | O | SER | I | 186 | 3.848 | 22.576 | 50.551 | 1.00 | 142.19 | O |
| ATOM | 18755 | N | ASN | I | 187 | 4.326 | 24.785 | 50.580 | 1.00 | 148.57 | 5 |
| ATOM | 18757 | CA | ASN | I | 187 | 3.185 | 25.150 | 49.752 | 1.00 | 152.45 | C |
| ATOM | 18759 | CB | ASN | I | 187 | 3.600 | 26.104 | 48.617 | 1.00 | 151.96 | C |
| ATOM | 18762 | CG | ASN | I | 187 | 5.065 | 25.948 | 48.205 | 1.00 | 152.00 | C |
| ATOM | 18763 | OD1 | ASN | I | 187 | 5.980 | 26.360 | 48.924 | 1.00 | 138.98 | O |
| ATOM | 18764 | ND2 | ASN | I | 187 | 5.288 | 25.380 | 47.021 | 1.00 | 147.64 | N |
| ATOM | 18767 | C | ASN | I | 187 | 2.539 | 23.855 | 49.231 | 1.00 | 156.10 | C |
| ATOM | 18768 | O | ASN | I | 187 | 1.868 | 23.147 | 49.994 | 1.00 | 156.29 | O |
| ATOM | 18769 | N | VAL | I | 188 | 2.794 | 23.509 | 47.969 | 1.00 | 158.74 | N |
| ATOM | 18771 | CA | VAL | I | 188 | 1.718 | 23.210 | 47.021 | 1.00 | 157.50 | C |
| ATOM | 18773 | CB | VAL | I | 108 | 1.693 | 21.698 | 46.618 | 1.00 | 154.28 | C |
| ATOM | 18775 | CG1 | VAL | I | 188 | 1.253 | 21.533 | 45.157 | 1.00 | 149.03 | C |
| ATOM | 18779 | CG2 | VAL | I | 188 | 3.068 | 21.050 | 46.827 | 1.00 | 146.04 | C |
| ATOM | 18783 | C | VAL | I | 188 | 0.387 | 23.682 | 47.643 | 1.00 | 160.91 | C |
| ATOM | 18784 | O | VAL | I | 188 | −0.389 | 22.867 | 48.148 | 1.00 | 162.30 | O |
| ATOM | 18785 | N | ASP | I | 189 | 0.155 | 25.002 | 47.635 | 1.00 | 161.27 | N |
| ATOM | 18707 | CA | ASP | I | 189 | −0.742 | 25.683 | 48.585 | 1.00 | 158.74 | C |
| ATOM | 18789 | CB | ASP | I | 189 | −0.378 | 25.337 | 50.037 | 1.00 | 157.93 | C |
| ATOM | 18792 | CG | ASP | I | 189 | −1.432 | 24.485 | 50.723 | 1.00 | 151.23 | C |
| ATOM | 18793 | OD1 | ASP | I | 189 | −1.086 | 23.749 | 51.670 | 1.00 | 134.33 | O |
| ATOM | 18794 | OD2 | ASP | I | 109 | −2.634 | 24.480 | 50.385 | 1.00 | 146.37 | O |
| ATOM | 18795 | C | ASP | I | 189 | −0.710 | 27.209 | 48.427 | 1.00 | 157.81 | C |
| ATOM | 18796 | O | ASP | I | 189 | 0.271 | 27.761 | 47.925 | 1.00 | 157.85 | O |
| ATOM | 18797 | N | LYS | I | 190 | −1.777 | 27.876 | 48.874 | 1.00 | 156.90 | N |
| ATOM | 18799 | CA | LYS | I | 190 | −1.765 | 29.316 | 49.169 | 1.00 | 155.88 | C |
| ATOM | 18801 | CB | LYS | I | 190 | −3.193 | 29.840 | 49.453 | 1.00 | 155.72 | C |
| ATOM | 18804 | CG | LYS | I | 190 | −3.291 | 31.256 | 50.078 | 1.00 | 150.55 | C |
| ATOM | 18807 | CD | LYS | I | 190 | −4.717 | 31.843 | 50.080 | 1.00 | 140.67 | C |
| ATOM | 18810 | CE | LYS | I | 190 | −5.767 | 30.914 | 50.714 | 1.00 | 135.80 | C |
| ATOM | 18813 | NZ | LYS | I | 190 | −5.446 | 30.513 | 52.119 | 1.00 | 114.99 | 5 |
| ATOM | 18817 | C | LYS | I | 190 | −0.821 | 29.625 | 50.339 | 1.00 | 155.11 | C |
| ATOM | 18818 | O | LYS | I | 190 | −1.218 | 29.589 | 51.508 | 1.00 | 156.23 | O |
| ATOM | 18819 | N | GLU | I | 191 | 0.437 | 29.907 | 50.004 | 1.00 | 151.66 | N |
| ATOM | 18821 | CA | GLU | I | 191 | 1.365 | 30.606 | 50.889 | 1.00 | 145.56 | C |
| ATOM | 18823 | CB | GLU | I | 191 | 1.753 | 29.695 | 52.063 | 1.00 | 148.22 | C |
| ATOM | 18826 | CG | GLU | I | 191 | 0.683 | 29.533 | 53.143 | 1.00 | 153.56 | C |
| ATOM | 18829 | CD | GLU | I | 191 | 0.153 | 28.106 | 53.279 | 1.00 | 158.56 | C |
| ATOM | 18830 | OE1 | GLU | I | 191 | −0.823 | 27.902 | 54.039 | 1.00 | 148.73 | O |
| ATOM | 18831 | OE2 | GLU | I | 191 | 0.707 | 27.180 | 52.641 | 1.00 | 160.83 | O |
| ATOM | 18832 | C | GLU | I | 191 | 2.586 | 30.993 | 50.042 | 1.00 | 137.97 | C |
| ATOM | 18833 | O | GLU | I | 191 | 2.441 | 31.274 | 48.850 | 1.00 | 132.36 | O |
| ATOM | 18834 | N | GLU | I | 192 | 3.782 | 30.968 | 50.630 | 1.00 | 133.17 | N |
| ATOM | 18836 | CA | GLU | I | 192 | 4.573 | 32.187 | 50.817 | 1.00 | 129.14 | C |
| ATOM | 18838 | CB | GLU | I | 192 | 4.006 | 33.291 | 49.902 | 1.00 | 132.54 | C |
| ATOM | 18841 | CG | GLU | I | 192 | 4.759 | 34.625 | 49.823 | 1.00 | 133.73 | C |
| ATOM | 18844 | CD | GLU | I | 192 | 4.095 | 35.652 | 48.890 | 1.00 | 131.66 | C |
| ATOM | 18845 | OE1 | GLU | I | 192 | 4.623 | 35.876 | 47.767 | 1.00 | 106.95 | O |
| ATOM | 18846 | OE2 | GLU | I | 192 | 3.053 | 36.255 | 49.274 | 1.00 | 114.63 | O |
| ATOM | 18847 | C | GLU | I | 192 | 4.540 | 32.610 | 52.303 | 1.00 | 124.44 | C |
| ATOM | 18848 | O | GLU | I | 192 | 5.154 | 33.611 | 52.697 | 1.00 | 117.72 | O |
| ATOM | 18849 | N | GLU | I | 193 | 3.775 | 31.872 | 53.112 | 1.00 | 117.25 | N |
| ATOM | 18851 | CA | GLU | I | 193 | 4.147 | 31.566 | 54.494 | 1.00 | 108.59 | C |
| ATOM | 18853 | CB | GLU | I | 193 | 3.272 | 32.323 | 55.495 | 1.00 | 111.52 | C |
| ATOM | 18856 | CG | GLU | I | 193 | 3.043 | 33.764 | 55.079 | 1.00 | 113.81 | C |
| ATOM | 18859 | CD | GLU | I | 193 | 1.746 | 33.894 | 54.307 | 1.00 | 123.55 | C |
| ATOM | 18860 | OE1 | GLU | I | 193 | 0.936 | 34.762 | 54.680 | 1.00 | 124.83 | O |
| ATOM | 18861 | OE2 | GLU | I | 193 | 1.489 | 33.093 | 53.376 | 1.00 | 125.35 | O |
| ATOM | 18862 | C | GLU | I | 193 | 4.219 | 30.052 | 54.754 | 1.00 | 97.95 | C |
| ATOM | 18863 | O | GLU | I | 193 | 3.461 | 29.439 | 55.517 | 1.00 | 86.88 | O |
| ATOM | 18864 | N | ALA | I | 194 | 5.263 | 29.514 | 54.140 | 1.00 | 89.20 | N |
| ATOM | 18866 | CA | ALA | I | 194 | 5.535 | 28.102 | 54.010 | 1.00 | 80.33 | C |
| ATOM | 18868 | CB | ALA | I | 194 | 5.772 | 27.788 | 52.540 | 1.00 | 77.89 | C |
| ATOM | 18872 | C | ALA | I | 194 | 6.819 | 27.911 | 54.796 | 1.00 | 71.79 | C |
| ATOM | 18873 | O | ALA | I | 194 | 7.357 | 20.892 | 55.315 | 1.00 | 63.01 | O |
| ATOM | 18874 | N | VAL | I | 195 | 7.326 | 26.677 | 54.834 | 1.00 | 62.01 | N |
| ATOM | 18876 | CA | VAL | I | 195 | 8.566 | 26.385 | 55.546 | 1.00 | 59.85 | C |
| ATOM | 18878 | CB | VAL | I | 195 | 8.320 | 25.865 | 56.998 | 1.00 | 54.99 | C |
| ATOM | 18880 | CG1 | VAL | I | 195 | 9.644 | 25.584 | 57.689 | 1.00 | 50.21 | C |
| ATOM | 18884 | CG2 | VAL | I | 195 | 7.528 | 26.875 | 57.831 | 1.00 | 56.13 | C |
| ATOM | 18888 | C | VAL | I | 195 | 9.450 | 25.410 | 54.764 | 1.00 | 60.18 | C |
| ATOM | 18889 | O | VAL | I | 195 | 9.132 | 24.231 | 54.639 | 1.00 | 62.08 | O |
| ATOM | 18890 | N | THR | I | 196 | 10.558 | 25.901 | 54.222 | 1.00 | 61.45 | N |
| ATOM | 18892 | CA | THR | I | 196 | 11.412 | 25.034 | 53.439 | 1.00 | 66.13 | C |
| ATOM | 18894 | CB | THR | I | 196 | 11.514 | 25.484 | 51.977 | 1.00 | 63.91 | C |
| ATOM | 18896 | OG1 | THR | I | 196 | 11.675 | 26.898 | 51.915 | 1.00 | 62.85 | O |
| ATOM | 18898 | CG2 | THR | I | 196 | 10.210 | 25.239 | 51.259 | 1.00 | 72.94 | C |
| ATOM | 18902 | C | THR | I | 196 | 12.798 | 24.875 | 54.042 | 1.00 | 69.35 | C |
| ATOM | 18903 | O | THR | I | 196 | 13.450 | 25.837 | 54.464 | 1.00 | 66.20 | O |
| ATOM | 18904 | N | ILE | I | 197 | 13.238 | 23.621 | 54.021 | 1.00 | 65.53 | N |
| ATOM | 18906 | CA | ILE | I | 197 | 14.536 | 23.254 | 54.517 | 1.00 | 59.47 | C |
| ATOM | 18908 | CB | ILE | I | 197 | 14.330 | 22.237 | 55.633 | 1.00 | 61.69 | C |
| ATOM | 18910 | CG1 | ILE | I | 197 | 13.468 | 22.907 | 56.699 | 1.00 | 50.77 | C |
| ATOM | 18913 | CD1 | ILE | I | 197 | 12.863 | 21.960 | 57.667 | 1.00 | 49.54 | C |
| ATOM | 18917 | CG2 | ILE | I | 197 | 15.660 | 21.775 | 56.219 | 1.00 | 57.75 | C |
| ATOM | 18921 | C | ILE | I | 197 | 15.273 | 22.684 | 53.345 | 1.00 | 55.52 | C |
| ATOM | 18922 | O | ILE | I | 197 | 14.672 | 21.934 | 52.593 | 1.00 | 61.32 | O |
| ATOM | 18923 | N | GLU | I | 198 | 16.499 | 23.170 | 53.133 | 1.00 | 65.40 | N |
| ATOM | 18925 | CA | GLU | I | 198 | 17.547 | 22.528 | 52.322 | 1.00 | 65.63 | C |
| ATOM | 18927 | CB | GLU | I | 198 | 18.352 | 23.547 | 51.503 | 1.00 | 61.71 | C |
| ATOM | 18930 | CG | GLU | I | 198 | 17.523 | 24.682 | 50.900 | 1.00 | 84.49 | C |
| ATOM | 18933 | CD | GLU | I | 198 | 17.260 | 24.508 | 49.412 | 1.00 | 97.11 | C |
| ATOM | 18934 | OE1 | GLU | I | 198 | 17.466 | 25.487 | 48.658 | 1.00 | 98.86 | O |
| ATOM | 18935 | OE2 | GLU | I | 198 | 16.831 | 23.401 | 49.003 | 1.00 | 96.76 | O |
| ATOM | 18936 | C | GLU | I | 198 | 18.529 | 21.917 | 53.273 | 1.00 | 64.49 | C |
| ATOM | 18937 | O | GLU | I | 198 | 19.413 | 22.612 | 53.729 | 1.00 | 65.10 | O |
| ATOM | 18938 | N | MET | I | 199 | 18.392 | 20.633 | 53.573 | 1.00 | 71.69 | N |
| ATOM | 18940 | CA | MET | I | 199 | 19.357 | 19.984 | 54.454 | 1.00 | 73.40 | C |
| ATOM | 18942 | CB | MET | I | 199 | 18.680 | 18.895 | 55.280 | 1.00 | 75.16 | C |
| ATOM | 18945 | CG | MET | I | 199 | 18.862 | 19.063 | 56.784 | 1.00 | 84.68 | C |
| ATOM | 18948 | SD | MET | I | 199 | 19.998 | 17.819 | 57.435 | 1.00 | 100.58 | S |
| ATOM | 18949 | CE | MET | I | 199 | 19.735 | 16.459 | 56.286 | 1.00 | 100.24 | C |
| ATOM | 18953 | C | MET | I | 199 | 20.526 | 19.423 | 53.647 | 1.00 | 71.03 | C |
| ATOM | 18954 | O | MET | I | 199 | 20.376 | 19.122 | 52.464 | 1.00 | 76.92 | O |
| ATOM | 18955 | N | ASN | I | 200 | 21.704 | 19.376 | 54.262 | 1.00 | 69.35 | N |
| ATOM | 18957 | CA | ASN | I | 200 | 22.835 | 18.576 | 53.785 | 1.00 | 65.96 | C |
| ATOM | 18959 | CB | ASN | I | 200 | 24.049 | 19.433 | 53.422 | 1.00 | 61.30 | C |
| ATOM | 18962 | CG | ASN | I | 200 | 23.855 | 20.191 | 52.142 | 1.00 | 64.22 | C |
| ATOM | 18963 | OD1 | ASN | I | 200 | 22.761 | 20.236 | 51.601 | 1.00 | 82.87 | O |
| ATOM | 18964 | ND2 | ASN | I | 200 | 24.915 | 20.809 | 51.651 | 1.00 | 87.09 | N |
| ATOM | 18967 | C | ASN | I | 200 | 23.266 | 17.674 | 54.917 | 1.00 | 67.18 | C |
| ATOM | 18968 | O | ASN | I | 200 | 23.678 | 16.545 | 54.709 | 1.00 | 71.20 | O |
| ATOM | 18969 | N | GLU | I | 201 | 23.208 | 18.213 | 56.125 | 1.00 | 71.28 | N |
| ATOM | 18971 | CA | GLU | I | 201 | 23.428 | 17.428 | 57.323 | 1.00 | 67.49 | C |
| ATOM | 18973 | CB | GLU | I | 201 | 24.916 | 17.225 | 57.549 | 1.00 | 68.37 | C |
| ATOM | 18976 | CG | GLU | I | 201 | 25.791 | 18.345 | 57.010 | 1.00 | 71.87 | C |
| ATOM | 18979 | CD | GLU | I | 201 | 27.171 | 18.319 | 57.647 | 1.00 | 103.26 | C |
| ATOM | 18980 | OE1 | GLU | I | 201 | 27.357 | 17.694 | 58.738 | 1.00 | 85.16 | O |
| ATOM | 18981 | OE2 | GLU | I | 201 | 28.878 | 18.915 | 57.021 | 1.00 | 117.73 | O |
| ATOM | 18982 | C | GLU | I | 201 | 22.828 | 18.186 | 58.492 | 1.00 | 69.90 | C |
| ATOM | 18983 | O | GLU | I | 201 | 22.817 | 19.424 | 58.554 | 1.00 | 73.68 | O |
| ATOM | 18984 | N | PRO | I | 202 | 22.304 | 17.435 | 59.438 | 1.00 | 65.80 | N |
| ATOM | 18985 | CA | PRO | I | 202 | 21.353 | 18.029 | 60.362 | 1.00 | 63.18 | C |
| ATOM | 18987 | CB | PRO | I | 202 | 20.705 | 16.815 | 61.014 | 1.00 | 63.35 | C |
| ATOM | 18990 | CG | PRO | I | 202 | 21.304 | 15.637 | 60.278 | 1.00 | 64.73 | C |
| ATOM | 18993 | CD | PRO | I | 202 | 22.666 | 16.067 | 59.827 | 1.00 | 63.01 | C |
| ATOM | 18996 | C | PRO | I | 202 | 22.196 | 18.826 | 61.329 | 1.00 | 61.20 | C |
| ATOM | 18997 | O | PRO | I | 202 | 23.283 | 18.392 | 61.704 | 1.00 | 62.99 | O |
| ATOM | 18998 | N | VAL | I | 203 | 21.719 | 20.007 | 61.678 | 1.00 | 61.17 | N |
| ATOM | 19000 | CA | VAL | I | 203 | 22.409 | 20.813 | 62.658 | 1.00 | 62.57 | C |
| ATOM | 19002 | CB | VAL | I | 203 | 22.641 | 22.167 | 62.084 | 1.00 | 63.24 | C |
| ATOM | 19004 | CG1 | VAL | I | 203 | 23.790 | 22.807 | 62.792 | 1.00 | 70.07 | C |
| ATOM | 19008 | CG2 | VAL | I | 203 | 22.947 | 21.976 | 60.615 | 1.00 | 74.85 | C |
| ATOM | 19012 | C | VAL | I | 203 | 21.677 | 20.904 | 63.980 | 1.00 | 58.72 | C |
| ATOM | 19013 | O | VAL | I | 203 | 20.471 | 20.711 | 64.041 | 1.00 | 62.93 | O |
| ATOM | 19014 | N | GLN | I | 204 | 22.419 | 21.103 | 65.061 | 1.00 | 57.07 | N |
| ATOM | 19016 | CA | GLN | I | 204 | 21.781 | 21.523 | 66.296 | 1.00 | 61.74 | C |
| ATOM | 19018 | CB | GLN | I | 204 | 21.383 | 20.350 | 67.196 | 1.00 | 59.97 | C |
| ATOM | 19021 | CG | GLN | I | 204 | 21.590 | 20.663 | 68.699 | 1.00 | 68.76 | C |
| ATOM | 19024 | CD | GLN | I | 204 | 20.995 | 19.606 | 69.632 | 1.00 | 65.97 | C |
| ATOM | 19025 | OE1 | GLN | I | 204 | 19.896 | 19.108 | 69.399 | 1.00 | 61.18 | O |
| ATOM | 19026 | NE2 | GLN | I | 204 | 21.726 | 19.263 | 70.680 | 1.00 | 67.65 | N |
| ATOM | 19029 | C | GLN | I | 204 | 22.640 | 22.536 | 67.026 | 1.00 | 57.54 | C |
| ATOM | 19030 | O | GLN | I | 204 | 23.642 | 22.197 | 67.665 | 1.00 | 59.97 | O |
| ATOM | 19031 | N | LEU | I | 205 | 22.241 | 23.797 | 66.905 | 1.00 | 52.28 | N |
| ATOM | 19033 | CA | LEU | I | 205 | 23.066 | 24.877 | 67.417 | 1.00 | 52.57 | C |
| ATOM | 19035 | CB | LEU | I | 205 | 23.493 | 25.786 | 66.285 | 1.00 | 53.37 | C |
| ATOM | 19038 | CG | LEU | I | 205 | 24.564 | 25.170 | 65.386 | 1.00 | 63.47 | C |
| ATOM | 19040 | CD1 | LEU | I | 205 | 24.244 | 25.522 | 63.954 | 1.00 | 64.10 | C |
| ATOM | 19044 | CD2 | LEU | I | 205 | 25.940 | 25.719 | 65.762 | 1.00 | 78.21 | C |
| ATOM | 19048 | C | LEU | I | 205 | 22.328 | 25.679 | 68.456 | 1.00 | 42.70 | C |
| ATOM | 19049 | O | LEU | I | 205 | 21.119 | 25.765 | 68.409 | 1.00 | 50.51 | O |
| ATOM | 19050 | N | THR | I | 206 | 23.079 | 26.236 | 69.392 | 1.00 | 46.39 | N |
| ATOM | 19052 | CA | THR | I | 206 | 22.552 | 27.022 | 70.512 | 1.00 | 47.70 | C |
| ATOM | 19054 | CB | THR | I | 206 | 23.152 | 26.492 | 71.823 | 1.00 | 46.46 | C |
| ATOM | 19056 | OG1 | THR | I | 206 | 22.632 | 25.190 | 72.117 | 1.00 | 53.73 | O |
| ATOM | 19058 | CG2 | THR | I | 206 | 22.773 | 27.358 | 72.986 | 1.00 | 41.04 | C |
| ATOM | 19062 | C | THR | I | 206 | 22.944 | 28.493 | 70.385 | 1.00 | 45.21 | C |
| ATOM | 19063 | O | THR | I | 206 | 24.136 | 28.840 | 70.324 | 1.00 | 38.81 | O |
| ATOM | 19064 | N | PHE | I | 207 | 21.933 | 29.351 | 70.357 | 1.00 | 40.65 | N |
| ATOM | 19066 | CA | PHE | I | 207 | 22.170 | 30.791 | 70.287 | 1.00 | 40.87 | C |
| ATOM | 19068 | CB | PHE | I | 207 | 21.632 | 31.330 | 68.963 | 1.00 | 35.37 | C |
| ATOM | 19071 | CG | PHE | I | 207 | 22.234 | 30.664 | 67.763 | 1.00 | 28.83 | C |
| ATOM | 19072 | CD1 | PHE | I | 207 | 23.428 | 31.093 | 67.245 | 1.00 | 48.65 | C |
| ATOM | 19074 | CE1 | PHE | I | 207 | 23.987 | 30.471 | 66.165 | 1.00 | 36.70 | C |
| ATOM | 19076 | CZ | PHE | I | 207 | 23.330 | 29.437 | 65.585 | 1.00 | 52.73 | C |
| ATOM | 19078 | CE2 | PHE | I | 207 | 22.162 | 28.981 | 66.120 | 1.00 | 27.45 | C |
| ATOM | 19080 | CD2 | PHE | I | 207 | 21.648 | 29.569 | 67.210 | 1.00 | 33.92 | C |
| ATOM | 19082 | C | PHE | I | 207 | 21.536 | 31.537 | 71.455 | 1.00 | 39.18 | C |
| ATOM | 19083 | O | PHE | I | 207 | 20.713 | 31.001 | 72.183 | 1.00 | 48.65 | O |
| ATOM | 19084 | N | ALA | I | 208 | 21.879 | 32.811 | 71.566 | 1.00 | 39.55 | N |
| ATOM | 19086 | CA | ALA | I | 208 | 21.351 | 33.710 | 72.582 | 1.00 | 34.75 | C |
| ATOM | 19088 | CB | ALA | I | 208 | 22.437 | 34.724 | 72.973 | 1.00 | 39.78 | C |
| ATOM | 19092 | C | ALA | I | 208 | 20.138 | 34.459 | 72.059 | 1.00 | 34.03 | C |
| ATOM | 19093 | O | ALA | I | 208 | 20.178 | 35.134 | 71.052 | 1.00 | 36.57 | O |
| ATOM | 19094 | N | LEU | I | 209 | 19.034 | 34.363 | 72.771 | 1.00 | 41.93 | N |
| ATOM | 19096 | CA | LEU | I | 209 | 17.768 | 34.799 | 72.225 | 1.00 | 38.17 | C |
| ATOM | 19098 | CB | LEU | I | 209 | 16.624 | 34.319 | 73.099 | 1.00 | 32.80 | C |
| ATOM | 19101 | CG | LEU | I | 209 | 16.175 | 32.941 | 72.662 | 1.00 | 39.81 | C |
| ATOM | 19103 | CD1 | LEU | I | 209 | 15.344 | 32.295 | 73.800 | 1.00 | 39.81 | C |
| ATOM | 19107 | CD2 | LEU | I | 209 | 15.423 | 33.134 | 71.334 | 1.00 | 36.54 | C |
| ATOM | 19111 | C | LEU | I | 209 | 17.748 | 36.301 | 72.138 | 1.00 | 41.10 | C |
| ATOM | 19112 | O | LEU | I | 209 | 17.029 | 36.888 | 71.339 | 1.00 | 54.51 | O |
| ATOM | 19113 | N | ARG | I | 210 | 18.541 | 36.939 | 72.970 | 1.00 | 47.14 | N |
| ATOM | 19115 | CA | ARG | I | 210 | 18.400 | 38.372 | 73.161 | 1.00 | 47.72 | C |
| ATOM | 19117 | CB | ARG | I | 210 | 19.217 | 38.779 | 74.379 | 1.00 | 48.33 | C |
| ATOM | 19120 | CG | ARG | I | 210 | 19.929 | 40.091 | 74.287 | 1.00 | 62.15 | C |
| ATOM | 19123 | CD | ARG | I | 210 | 20.478 | 40.496 | 75.635 | 1.00 | 78.84 | C |
| ATOM | 19126 | NE | ARG | I | 210 | 21.455 | 39.492 | 76.029 | 1.00 | 78.87 | N |
| ATOM | 19128 | CZ | ARG | I | 210 | 22.748 | 39.707 | 75.913 | 1.00 | 77.76 | C |
| ATOM | 19129 | NH1 | ARG | I | 210 | 23.142 | 40.902 | 75.462 | 1.00 | 62.99 | N |
| ATOM | 19132 | NH2 | ARG | I | 210 | 23.616 | 38.752 | 76.246 | 1.00 | 60.18 | N |
| ATOM | 19135 | C | ARG | I | 210 | 18.900 | 39.072 | 71.910 | 1.00 | 44.66 | C |
| ATOM | 19136 | O | ARG | I | 210 | 18.534 | 40.199 | 71.635 | 1.00 | 53.77 | O |
| ATOM | 19137 | N | TYR | I | 211 | 19.690 | 38.390 | 71.097 | 1.00 | 44.79 | N |
| ATOM | 19139 | CA | TYR | I | 211 | 20.277 | 39.046 | 69.945 | 1.00 | 38.70 | C |
| ATOM | 19141 | CB | TYR | I | 211 | 21.638 | 38.413 | 69.705 | 1.00 | 39.80 | C |
| ATOM | 19144 | CG | TYR | I | 211 | 22.748 | 38.925 | 70.610 | 1.00 | 46.91 | C |
| ATOM | 19145 | CD1 | TYR | I | 211 | 23.055 | 38.275 | 71.795 | 1.00 | 43.30 | C |
| ATOM | 19147 | CE1 | TYR | I | 211 | 24.096 | 38.701 | 72.595 | 1.00 | 48.30 | C |
| ATOM | 19149 | CZ | TYR | I | 211 | 24.839 | 39.798 | 72.239 | 1.00 | 25.19 | C |
| ATOM | 19150 | OH | TYR | I | 211 | 25.906 | 40.186 | 73.037 | 1.00 | 43.92 | O |
| ATOM | 19152 | CE2 | TYR | I | 211 | 24.560 | 40.455 | 71.060 | 1.00 | 41.48 | C |
| ATOM | 19154 | CD2 | TYR | I | 211 | 23.534 | 40.014 | 70.244 | 1.00 | 30.93 | C |
| ATOM | 19156 | C | TYR | I | 211 | 19.385 | 38.848 | 68.725 | 1.00 | 41.17 | C |
| ATOM | 19157 | O | TYR | I | 211 | 19.233 | 39.691 | 67.817 | 1.00 | 31.18 | O |
| ATOM | 19158 | N | LEU | I | 212 | 18.775 | 37.677 | 68.698 | 1.00 | 39.67 | N |
| ATOM | 19160 | CA | LEU | I | 212 | 17.825 | 37.412 | 67.646 | 1.00 | 37.57 | C |
| ATOM | 19162 | CB | LEU | I | 212 | 17.331 | 35.977 | 67.822 | 1.00 | 42.72 | C |
| ATOM | 19165 | CG | LEU | I | 212 | 18.355 | 34.836 | 67.656 | 1.00 | 40.50 | C |
| ATOM | 19167 | CD1 | LEU | I | 212 | 17.843 | 33.528 | 68.295 | 1.00 | 35.96 | C |
| ATOM | 19171 | CD2 | LEU | I | 212 | 18.645 | 34.566 | 66.197 | 1.00 | 30.08 | C |
| ATOM | 19175 | C | LEU | I | 212 | 16.680 | 38.456 | 67.677 | 1.00 | 41.59 | C |
| ATOM | 19176 | O | LEU | I | 212 | 16.031 | 38.742 | 66.672 | 1.00 | 34.57 | O |
| ATOM | 19177 | N | ASN | I | 213 | 16.415 | 39.032 | 60.843 | 1.00 | 40.81 | N |
| ATOM | 19179 | CA | ASN | I | 213 | 15.208 | 39.799 | 69.005 | 1.00 | 35.13 | C |
| ATOM | 19181 | CB | ASN | I | 213 | 14.717 | 39.780 | 70.444 | 1.00 | 39.59 | C |
| ATOM | 19184 | CG | ASN | I | 213 | 13.803 | 38.614 | 70.738 | 1.00 | 32.26 | C |
| ATOM | 19185 | OD1 | ASN | I | 213 | 12.955 | 38.238 | 69.935 | 1.00 | 49.76 | O |
| ATOM | 19186 | ND2 | ASN | I | 213 | 13.998 | 38.010 | 71.888 | 1.00 | 44.56 | N |
| ATOM | 19189 | C | ASN | I | 213 | 15.879 | 41.193 | 68.596 | 1.00 | 40.43 | C |
| ATOM | 19190 | O | ASN | I | 213 | 14.715 | 42.056 | 68.428 | 1.00 | 45.11 | O |
| ATOM | 19191 | N | PHE | I | 214 | 16.870 | 41.403 | 60.382 | 1.00 | 38.92 | N |
| ATOM | 19193 | CA | PHE | I | 214 | 17.267 | 42.613 | 67.678 | 1.00 | 41.63 | C |
| ATOM | 19195 | CB | PHE | I | 214 | 18.692 | 43.045 | 67.997 | 1.00 | 41.55 | C |
| ATOM | 19198 | CG | PHE | I | 214 | 18.830 | 43.651 | 69.363 | 1.00 | 49.06 | C |
| ATOM | 19199 | CD1 | PHE | I | 214 | 18.702 | 45.015 | 69.547 | 1.00 | 51.19 | C |
| ATOM | 19201 | CE1 | PHE | I | 214 | 18.832 | 45.573 | 70.800 | 1.00 | 42.84 | C |
| ATOM | 19203 | CZ | PHE | I | 214 | 19.067 | 44.776 | 71.879 | 1.00 | 44.55 | C |
| ATOM | 19205 | CE2 | PHE | I | 214 | 19.188 | 43.399 | 71.717 | 1.00 | 41.98 | C |
| ATOM | 19207 | CD2 | PHE | I | 214 | 19.059 | 42.848 | 70.471 | 1.00 | 51.16 | C |
| ATOM | 19209 | C | PHE | I | 214 | 17.105 | 42.394 | 66.206 | 1.00 | 36.97 | C |
| ATOM | 19210 | O | PHE | I | 214 | 16.670 | 43.315 | 65.533 | 1.00 | 35.57 | O |
| ATOM | 19211 | N | PHE | I | 215 | 17.439 | 41.186 | 65.747 | 1.00 | 33.45 | N |
| ATOM | 19213 | CA | PHE | I | 215 | 17.425 | 40.879 | 64.330 | 1.00 | 31.79 | C |
| ATOM | 19215 | CB | PHE | I | 215 | 17.962 | 39.473 | 64.035 | 1.00 | 39.93 | C |
| ATOM | 19218 | CG | PHE | I | 215 | 19.370 | 39.214 | 64.521 | 1.00 | 36.17 | C |
| ATOM | 19219 | CD1 | PHE | I | 215 | 20.154 | 40.228 | 64.994 | 1.00 | 34.36 | C |
| ATOM | 19221 | CE1 | PHE | I | 215 | 21.446 | 39.980 | 65.424 | 1.00 | 35.66 | C |
| ATOM | 19223 | CZ | PHE | I | 215 | 21.952 | 38.697 | 65.411 | 1.00 | 40.84 | C |
| ATOM | 19225 | CE2 | PHE | I | 215 | 21.199 | 37.686 | 64.885 | 1.00 | 38.47 | C |
| ATOM | 19227 | CD2 | PHE | I | 215 | 19.942 | 37.967 | 64.368 | 1.00 | 31.31 | C |
| ATOM | 19229 | C | PHE | I | 215 | 15.992 | 40.982 | 63.815 | 1.00 | 35.98 | C |
| ATOM | 19230 | O | PHE | I | 215 | 15.774 | 41.418 | 62.682 | 1.00 | 42.25 | O |
| ATOM | 19231 | N | THR | I | 216 | 15.009 | 40.600 | 64.629 | 1.00 | 34.53 | N |
| ATOM | 19233 | CA | THR | I | 216 | 13.644 | 40.534 | 64.125 | 1.00 | 35.21 | C |
| ATOM | 19235 | CB | THR | I | 216 | 12.746 | 39.739 | 65.054 | 1.00 | 39.18 | C |
| ATOM | 19237 | OG1 | THR | I | 216 | 12.912 | 40.195 | 66.399 | 1.00 | 41.56 | O |
| ATOM | 19239 | CG2 | THR | I | 216 | 13.100 | 38.276 | 65.053 | 1.00 | 38.23 | C |
| ATOM | 19243 | C | THR | I | 216 | 12.987 | 41.894 | 63.962 | 1.00 | 31.80 | C |
| ATOM | 19244 | O | THR | I | 216 | 11.897 | 41.972 | 63.434 | 1.00 | 44.66 | O |
| ATOM | 19245 | N | LYS | I | 217 | 13.616 | 42.971 | 64.403 | 1.00 | 29.55 | N |
| ATOM | 19247 | CA | LYS | I | 217 | 13.180 | 44.278 | 63.963 | 1.00 | 28.31 | C |
| ATOM | 19249 | CB | LYS | I | 217 | 13.957 | 45.397 | 64.641 | 1.00 | 32.37 | C |
| ATOM | 19252 | CG | LYS | I | 217 | 14.264 | 45.215 | 66.116 | 1.00 | 42.45 | C |
| ATOM | 19255 | CD | LYS | I | 217 | 13.816 | 46.401 | 66.950 | 1.00 | 56.08 | C |
| ATOM | 19258 | CE | LYS | I | 217 | 12.707 | 46.017 | 67.927 | 1.00 | 70.57 | C |
| ATOM | 19261 | NZ | LYS | I | 217 | 13.308 | 45.751 | 69.242 | 1.00 | 64.91 | N |
| ATOM | 19265 | C | LYS | I | 217 | 13.349 | 44.494 | 62.471 | 1.00 | 30.79 | C |
| ATOM | 19266 | O | LYS | I | 217 | 12.974 | 45.544 | 62.010 | 1.00 | 36.53 | O |
| ATOM | 19267 | N | ALA | I | 218 | 13.950 | 43.587 | 61.707 | 1.00 | 36.41 | N |
| ATOM | 19269 | CA | ALA | I | 218 | 13.950 | 43.764 | 60.259 | 1.00 | 35.56 | C |
| ATOM | 19271 | CB | ALA | I | 218 | 15.162 | 43.170 | 59.658 | 1.00 | 42.45 | C |
| ATOM | 19275 | C | ALA | I | 218 | 12.721 | 43.147 | 59.630 | 1.00 | 37.44 | C |
| ATOM | 19276 | O | ALA | I | 218 | 12.555 | 43.157 | 58.418 | 1.00 | 39.12 | O |
| ATOM | 19277 | N | THR | I | 219 | 11.811 | 42.673 | 60.465 | 1.00 | 43.88 | N |
| ATOM | 19279 | CA | THR | I | 219 | 10.611 | 42.012 | 59.957 | 1.00 | 45.11 | C |
| ATOM | 19281 | CB | THR | I | 219 | 9.742 | 41.526 | 61.095 | 1.00 | 40.30 | C |
| ATOM | 19283 | OG1 | THR | I | 219 | 10.488 | 40.587 | 61.871 | 1.00 | 41.07 | O |
| ATOM | 19285 | CG2 | THR | I | 219 | 8.655 | 40.677 | 60.536 | 1.00 | 34.24 | C |
| ATOM | 19289 | C | THR | I | 219 | 9.730 | 42.750 | 58.965 | 1.00 | 39.43 | C |
| ATOM | 19290 | O | THR | I | 219 | 9.114 | 42.139 | 58.104 | 1.00 | 50.30 | O |
| ATOM | 19291 | N | PRO | I | 220 | 9.563 | 44.041 | 59.157 | 1.00 | 31.43 | N |
| ATOM | 19292 | CA | PRO | I | 220 | 8.874 | 44.870 | 58.166 | 1.00 | 33.38 | C |
| ATOM | 19294 | CB | PRO | I | 220 | 9.106 | 46.295 | 58.686 | 1.00 | 23.72 | C |
| ATOM | 19297 | CG | PRO | I | 220 | 9.024 | 46.055 | 60.178 | 1.00 | 17.34 | C |
| ATOM | 19300 | CD | PRO | I | 220 | 9.815 | 44.769 | 60.406 | 1.00 | 31.06 | C |
| ATOM | 19303 | C | PRO | I | 220 | 9.376 | 44.697 | 56.751 | 1.00 | 26.65 | C |
| ATOM | 19304 | O | PRO | I | 220 | 8.690 | 45.108 | 55.844 | 1.00 | 42.34 | O |
| ATOM | 19305 | N | LEU | I | 221 | 10.592 | 44.228 | 56.559 | 1.00 | 32.54 | N |
| ATOM | 19307 | CA | LEU | I | 221 | 11.228 | 44.422 | 55.264 | 1.00 | 37.38 | C |
| ATOM | 19309 | CB | LEU | I | 221 | 12.724 | 44.650 | 55.452 | 1.00 | 38.24 | C |
| ATOM | 19312 | CG | LEU | I | 221 | 13.171 | 46.034 | 55.941 | 1.00 | 41.85 | C |
| ATOM | 19314 | CD1 | LEU | I | 221 | 14.596 | 45.988 | 56.518 | 1.00 | 24.81 | C |
| ATOM | 19318 | CD2 | LEU | I | 221 | 13.155 | 46.961 | 54.756 | 1.00 | 41.36 | C |
| ATOM | 19322 | C | LEU | I | 221 | 10.981 | 43.199 | 54.374 | 1.00 | 40.83 | C |
| ATOM | 19323 | O | LEU | I | 221 | 11.139 | 43.241 | 53.155 | 1.00 | 52.45 | O |
| ATOM | 19324 | N | SER | I | 222 | 10.538 | 42.113 | 54.986 | 1.00 | 38.31 | N |
| ATOM | 19326 | CA | SER | I | 222 | 10.234 | 40.911 | 54.256 | 1.00 | 41.87 | C |
| ATOM | 19328 | CB | SER | I | 222 | 11.509 | 40.129 | 53.991 | 1.00 | 49.95 | C |
| ATOM | 19331 | OG | SER | I | 222 | 11.212 | 38.948 | 53.253 | 1.00 | 74.15 | O |
| ATOM | 19333 | C | SER | I | 222 | 9.230 | 39.996 | 54.952 | 1.00 | 46.74 | C |
| ATOM | 19334 | O | SER | I | 222 | 9.198 | 39.856 | 56.182 | 1.00 | 43.13 | O |
| ATOM | 19335 | N | SER | I | 223 | 8.483 | 39.291 | 54.111 | 1.00 | 50.74 | N |
| ATOM | 19337 | CA | SER | I | 223 | 7.452 | 38.359 | 54.562 | 1.00 | 53.15 | C |
| ATOM | 19339 | CB | SER | I | 223 | 6.523 | 37.947 | 53.395 | 1.00 | 53.80 | C |
| ATOM | 19342 | OG | SER | I | 223 | 7.102 | 38.192 | 52.103 | 1.00 | 53.37 | O |
| ATOM | 19344 | C | SER | I | 223 | 8.168 | 37.145 | 55.139 | 1.00 | 52.22 | C |
| ATOM | 19345 | O | SER | I | 223 | 7.600 | 36.378 | 55.916 | 1.00 | 59.98 | O |
| ATOM | 19346 | N | THR | I | 224 | 9.417 | 36.958 | 54.735 | 1.00 | 45.82 | N |
| ATOM | 19348 | CA | THR | I | 224 | 10.035 | 35.652 | 54.846 | 1.00 | 50.27 | C |
| ATOM | 19350 | CB | THR | I | 224 | 10.002 | 34.938 | 53.465 | 1.00 | 47.96 | C |
| ATOM | 19352 | OG1 | THR | I | 224 | 10.975 | 33.888 | 53.424 | 1.00 | 68.70 | O |
| ATOM | 19354 | CG2 | THR | I | 224 | 10.519 | 35.862 | 52.380 | 1.00 | 65.70 | C |
| ATOM | 19358 | C | THR | I | 224 | 11.463 | 35.901 | 55.334 | 1.00 | 48.13 | C |
| ATOM | 19359 | O | THR | I | 224 | 12.043 | 36.953 | 55.027 | 1.00 | 48.41 | O |
| ATOM | 19360 | N | VAL | I | 225 | 12.003 | 34.958 | 56.110 | 1.00 | 41.68 | N |
| ATOM | 19362 | CA | VAL | I | 225 | 13.372 | 35.055 | 56.606 | 1.00 | 39.77 | C |
| ATOM | 19364 | CB | VAL | I | 225 | 13.398 | 35.408 | 58.100 | 1.00 | 48.48 | C |
| ATOM | 19366 | CG1 | VAL | I | 225 | 12.692 | 34.332 | 59.007 | 1.00 | 31.29 | C |
| ATOM | 19370 | CG2 | VAL | I | 225 | 14.829 | 35.640 | 58.528 | 1.00 | 52.98 | C |
| ATOM | 19374 | C | VAL | I | 225 | 14.143 | 33.759 | 56.391 | 1.00 | 41.61 | C |
| ATOM | 19375 | O | VAL | I | 225 | 13.574 | 32.681 | 56.456 | 1.00 | 41.60 | O |
| ATOM | 19376 | N | THR | I | 226 | 15.433 | 33.845 | 56.099 | 1.00 | 41.81 | N |
| ATOM | 19378 | CA | THR | I | 226 | 16.191 | 32.627 | 55.810 | 1.00 | 51.15 | C |
| ATOM | 19380 | CB | THR | I | 226 | 16.802 | 32.684 | 54.381 | 1.00 | 47.14 | C |
| ATOM | 19382 | OG1 | THR | I | 226 | 16.430 | 33.922 | 53.761 | 1.00 | 73.86 | O |
| ATOM | 19384 | CG2 | THR | I | 226 | 16.127 | 31.747 | 53.439 | 1.00 | 46.42 | C |
| ATOM | 19388 | C | THR | I | 226 | 17.252 | 32.433 | 56.895 | 1.00 | 47.84 | C |
| ATOM | 19389 | O | THR | I | 226 | 18.017 | 33.344 | 57.195 | 1.00 | 47.33 | O |
| ATOM | 19390 | N | LEU | I | 227 | 17.239 | 31.277 | 57.541 | 1.00 | 43.79 | N |
| ATOM | 19392 | CA | LEU | I | 227 | 18.218 | 30.989 | 58.583 | 1.00 | 47.81 | C |
| ATOM | 19394 | CB | LEU | I | 227 | 17.535 | 30.355 | 59.808 | 1.00 | 38.24 | C |
| ATOM | 19397 | CG | LEU | I | 227 | 16.319 | 31.074 | 60.403 | 1.00 | 54.77 | C |
| ATOM | 19399 | CD1 | LEU | I | 227 | 15.693 | 30.250 | 61.527 | 1.00 | 54.85 | C |
| ATOM | 19403 | CD2 | LEU | I | 227 | 16.632 | 32.485 | 60.931 | 1.00 | 59.16 | C |
| ATOM | 19407 | C | LEU | I | 227 | 19.260 | 30.036 | 57.996 | 1.00 | 46.17 | C |
| ATOM | 19408 | O | LEU | I | 227 | 18.940 | 28.921 | 57.640 | 1.00 | 58.88 | O |
| ATOM | 19409 | N | SER | I | 228 | 20.507 | 30.438 | 57.868 | 1.00 | 39.68 | N |
| ATOM | 19411 | CA | SER | I | 228 | 21.485 | 29.464 | 57.445 | 1.00 | 44.61 | C |
| ATOM | 19413 | CB | SER | I | 228 | 22.258 | 30.036 | 56.273 | 1.00 | 47.78 | C |
| ATOM | 19416 | OG | SER | I | 228 | 21.381 | 30.846 | 55.536 | 1.00 | 44.78 | O |
| ATOM | 19418 | C | SER | I | 228 | 22.452 | 29.056 | 58.565 | 1.00 | 51.99 | C |
| ATOM | 19419 | O | SER | I | 228 | 23.069 | 29.895 | 59.245 | 1.00 | 45.82 | O |
| ATOM | 19420 | N | MET | I | 229 | 22.614 | 27.747 | 58.715 | 1.00 | 51.06 | N |
| ATOM | 19422 | CA | MET | I | 229 | 23.255 | 27.193 | 59.897 | 1.00 | 57.86 | C |
| ATOM | 19424 | CB | MET | I | 229 | 22.212 | 26.383 | 60.671 | 1.00 | 60.32 | C |
| ATOM | 19427 | CG | MET | I | 229 | 21.729 | 27.085 | 61.921 | 1.00 | 62.82 | C |
| ATOM | 19430 | SD | MET | I | 229 | 20.061 | 26.646 | 62.343 | 1.00 | 68.66 | S |
| ATOM | 19431 | CE | MET | I | 229 | 19.188 | 27.013 | 60.842 | 1.00 | 82.09 | C |
| ATOM | 19435 | C | MET | I | 229 | 24.448 | 26.304 | 59.547 | 1.00 | 54.91 | C |
| ATOM | 19436 | O | MET | I | 229 | 24.405 | 25.547 | 58.595 | 1.00 | 63.80 | O |
| ATOM | 19437 | N | SER | I | 230 | 25.495 | 26.295 | 60.347 | 1.00 | 50.05 | N |
| ATOM | 19439 | CA | SER | I | 230 | 26.349 | 25.108 | 60.339 | 1.00 | 52.39 | C |
| ATOM | 19441 | CB | SER | I | 230 | 27.406 | 25.144 | 59.224 | 1.00 | 52.31 | C |
| ATOM | 19444 | OG | SER | I | 230 | 26.843 | 25.394 | 57.945 | 1.00 | 49.67 | O |
| ATOM | 19446 | C | SER | I | 230 | 27.064 | 25.078 | 61.657 | 1.00 | 51.19 | C |
| ATOM | 19447 | O | SER | I | 230 | 27.121 | 26.089 | 62.375 | 1.00 | 49.89 | O |
| ATOM | 19448 | N | ALA | I | 231 | 27.618 | 23.917 | 61.971 | 1.00 | 53.11 | N |
| ATOM | 19450 | CA | ALA | I | 231 | 28.277 | 23.763 | 63.262 | 1.00 | 60.12 | C |
| ATOM | 19452 | CB | ALA | I | 231 | 28.459 | 22.303 | 63.629 | 1.00 | 66.78 | C |
| ATOM | 19456 | C | ALA | I | 231 | 29.605 | 24.509 | 63.283 | 1.00 | 56.74 | C |
| ATOM | 19457 | O | ALA | I | 231 | 30.348 | 24.502 | 62.302 | 1.00 | 43.06 | O |
| ATOM | 19458 | N | ASP | I | 232 | 29.817 | 25.212 | 64.394 | 1.00 | 62.90 | N |
| ATOM | 19460 | CA | ASP | I | 232 | 30.910 | 26.166 | 64.562 | 1.00 | 72.96 | C |
| ATOM | 19462 | CB | ASP | I | 232 | 32.275 | 25.468 | 64.740 | 1.00 | 77.60 | C |
| ATOM | 19465 | CG | ASP | I | 232 | 32.166 | 24.146 | 65.473 | 1.00 | 83.51 | C |
| ATOM | 19466 | OD1 | ASP | I | 232 | 32.312 | 23.075 | 64.812 | 1.00 | 73.78 | O |
| ATOM | 19467 | OD2 | ASP | I | 232 | 31.919 | 24.127 | 66.706 | 1.00 | 69.36 | O |
| ATOM | 19468 | C | ASP | I | 232 | 31.007 | 27.125 | 63.396 | 1.00 | 68.22 | C |
| ATOM | 19469 | O | ASP | I | 232 | 32.100 | 27.558 | 63.025 | 1.00 | 67.48 | O |
| ATOM | 19470 | N | VAL | I | 233 | 29.870 | 27.561 | 62.886 | 1.00 | 66.28 | N |
| ATOM | 19472 | CA | VAL | I | 233 | 29.881 | 28.886 | 62.294 | 1.00 | 66.97 | C |
| ATOM | 19474 | CB | VAL | I | 233 | 30.002 | 28.789 | 60.763 | 1.00 | 69.41 | C |
| ATOM | 19476 | CG1 | VAL | I | 233 | 30.516 | 27.393 | 60.410 | 1.00 | 77.99 | C |
| ATOM | 19480 | CG2 | VAL | I | 233 | 28.678 | 29.088 | 60.071 | 1.00 | 65.82 | C |
| ATOM | 19484 | C | VAL | I | 233 | 28.747 | 29.766 | 62.786 | 1.00 | 57.97 | C |
| ATOM | 19485 | O | VAL | I | 233 | 27.733 | 29.260 | 63.263 | 1.00 | 60.87 | O |
| ATOM | 19486 | N | PRO | I | 234 | 28.954 | 31.080 | 62.714 | 1.00 | 57.07 | N |
| ATOM | 19487 | CA | PRO | I | 234 | 27.974 | 32.043 | 63.225 | 1.00 | 50.96 | C |
| ATOM | 19489 | CB | PRO | I | 234 | 28.645 | 33.401 | 62.998 | 1.00 | 51.11 | C |
| ATOM | 19492 | CG | PRO | I | 234 | 29.660 | 33.167 | 61.917 | 1.00 | 48.36 | C |
| ATOM | 19495 | CD | PRO | I | 234 | 30.100 | 31.749 | 62.070 | 1.00 | 56.27 | C |
| ATOM | 19498 | C | PRO | I | 234 | 26.739 | 31.897 | 62.384 | 1.00 | 45.88 | C |
| ATOM | 19499 | O | PRO | I | 234 | 26.894 | 31.603 | 61.199 | 1.00 | 35.93 | O |
| ATOM | 19500 | N | LEU | I | 235 | 25.570 | 32.028 | 63.009 | 1.00 | 46.79 | N |
| ATOM | 19502 | CA | LEU | I | 235 | 24.267 | 31.920 | 62.332 | 1.00 | 45.49 | C |
| ATOM | 19504 | CB | LEU | I | 235 | 23.142 | 31.927 | 63.363 | 1.00 | 46.96 | C |
| ATOM | 19507 | CG | LEU | I | 235 | 21.683 | 32.147 | 62.966 | 1.00 | 55.26 | C |
| ATOM | 19509 | CD1 | LEU | I | 235 | 21.218 | 31.092 | 61.989 | 1.00 | 65.53 | C |
| ATOM | 19513 | CD2 | LEU | I | 235 | 20.852 | 32.041 | 64.255 | 1.00 | 63.44 | C |
| ATOM | 19517 | C | LEU | I | 235 | 24.070 | 33.079 | 61.416 | 1.00 | 26.42 | C |
| ATOM | 19518 | O | LEU | I | 235 | 24.493 | 34.159 | 61.765 | 1.00 | 40.22 | O |
| ATOM | 19519 | N | VAL | I | 236 | 23.489 | 32.870 | 60.243 | 1.00 | 34.25 | N |
| ATOM | 19521 | CA | VAL | I | 236 | 22.974 | 33.998 | 59.437 | 1.00 | 39.63 | C |
| ATOM | 19523 | CB | VAL | I | 236 | 23.395 | 33.958 | 57.972 | 1.00 | 42.28 | C |
| ATOM | 19525 | CG1 | VAL | I | 236 | 23.361 | 35.359 | 67.400 | 1.00 | 52.06 | C |
| ATOM | 19529 | CG2 | VAL | I | 236 | 24.776 | 33.337 | 57.822 | 1.00 | 52.03 | C |
| ATOM | 19533 | C | VAL | I | 236 | 21.456 | 34.029 | 59.408 | 1.00 | 37.07 | C |
| ATOM | 19534 | O | VAL | I | 236 | 20.823 | 32.996 | 59.194 | 1.00 | 41.65 | O |
| ATOM | 19535 | N | VAL | I | 237 | 20.902 | 35.204 | 59.688 | 1.00 | 37.71 | N |
| ATOM | 19537 | CA | VAL | I | 237 | 19.471 | 35.489 | 59.581 | 1.00 | 37.85 | C |
| ATOM | 19539 | CB | VAL | I | 237 | 18.969 | 35.974 | 60.936 | 1.00 | 41.35 | C |
| ATOM | 19541 | CG1 | VAL | I | 237 | 17.517 | 36.385 | 60.859 | 1.00 | 35.04 | C |
| ATOM | 19545 | CG2 | VAL | I | 237 | 19.229 | 34.927 | 62.008 | 1.00 | 46.65 | C |
| ATOM | 19549 | C | VAL | I | 237 | 19.253 | 36.602 | 58.547 | 1.00 | 37.77 | C |
| ATOM | 19550 | O | VAL | I | 237 | 19.692 | 37.735 | 58.762 | 1.00 | 40.64 | O |
| ATOM | 19551 | N | GLU | I | 238 | 18.723 | 36.260 | 57.376 | 1.00 | 37.59 | N |
| ATOM | 19553 | CA | GLU | I | 238 | 18.744 | 37.163 | 56.226 | 1.00 | 39.27 | C |
| ATOM | 19555 | CB | GLU | I | 238 | 19.459 | 36.487 | 55.067 | 1.00 | 44.05 | C |
| ATOM | 19558 | CG | GLU | I | 238 | 19.028 | 36.933 | 53.680 | 1.00 | 50.81 | C |
| ATOM | 19561 | CD | GLU | I | 238 | 19.849 | 36.327 | 52.553 | 1.00 | 64.98 | C |
| ATOM | 19562 | OE1 | GLU | I | 238 | 19.720 | 35.113 | 52.281 | 1.00 | 69.35 | O |
| ATOM | 19563 | OE2 | GLU | I | 238 | 20.599 | 37.087 | 51.905 | 1.00 | 78.04 | O |
| ATOM | 19564 | C | GLU | I | 238 | 17.313 | 37.580 | 55.834 | 1.00 | 45.21 | C |
| ATOM | 19565 | O | GLU | I | 238 | 16.357 | 36.800 | 55.911 | 1.00 | 46.63 | O |
| ATOM | 19566 | N | TYR | I | 239 | 17.173 | 38.855 | 55.482 | 1.00 | 45.85 | N |
| ATOM | 19568 | CA | TYR | I | 239 | 15.901 | 39.491 | 55.161 | 1.00 | 40.72 | C |
| ATOM | 19570 | CB | TYR | I | 239 | 15.605 | 40.581 | 56.194 | 1.00 | 36.83 | C |
| ATOM | 19573 | CG | TYR | I | 239 | 15.265 | 40.032 | 57.556 | 1.00 | 40.71 | C |
| ATOM | 19574 | CD1 | TYR | I | 239 | 16.212 | 39.925 | 58.553 | 1.00 | 34.08 | C |
| ATOM | 19576 | CE1 | TYR | I | 239 | 15.866 | 39.417 | 59.834 | 1.00 | 47.43 | C |
| ATOM | 19578 | CZ | TYR | I | 239 | 14.569 | 39.023 | 60.109 | 1.00 | 18.29 | C |
| ATOM | 19579 | OH | TYR | I | 239 | 14.194 | 38.468 | 61.312 | 1.00 | 26.07 | O |
| ATOM | 19581 | CE2 | TYR | I | 239 | 13.638 | 39.097 | 59.114 | 1.00 | 26.56 | C |
| ATOM | 19583 | CD2 | TYR | I | 239 | 13.976 | 39.623 | 57.855 | 1.00 | 41.91 | C |
| ATOM | 19585 | C | TYR | I | 239 | 16.105 | 40.136 | 53.788 | 1.00 | 43.50 | C |
| ATOM | 19586 | O | TYR | I | 239 | 16.927 | 41.033 | 53.661 | 1.00 | 36.88 | O |
| ATOM | 19587 | N | LYS | I | 240 | 15.451 | 39.629 | 52.743 | 1.00 | 45.63 | N |
| ATOM | 19589 | CA | LYS | I | 240 | 15.630 | 40.195 | 51.408 | 1.00 | 40.66 | C |
| ATOM | 19591 | CB | LYS | I | 240 | 15.153 | 39.260 | 50.275 | 1.00 | 46.16 | C |
| ATOM | 19594 | CG | LYS | I | 240 | 16.209 | 38.131 | 49.868 | 1.00 | 73.96 | C |
| ATOM | 19597 | CD | LYS | I | 240 | 16.111 | 36.760 | 50.632 | 1.00 | 94.94 | C |
| ATOM | 19600 | CE | LYS | I | 240 | 14.761 | 36.509 | 51.389 | 1.00 | 103.56 | C |
| ATOM | 19603 | NZ | LYS | I | 240 | 14.818 | 35.724 | 52.674 | 1.00 | 75.03 | N |
| ATOM | 19607 | C | LYS | I | 240 | 14.844 | 41.474 | 51.431 | 1.00 | 35.24 | C |
| ATOM | 19608 | O | LYS | I | 240 | 13.726 | 41.498 | 51.927 | 1.00 | 37.97 | O |
| ATOM | 19609 | N | ILE | I | 241 | 15.473 | 42.558 | 50.991 | 1.00 | 37.34 | N |
| ATOM | 19611 | CA | ILE | I | 241 | 14.773 | 43.814 | 50.833 | 1.00 | 35.08 | C |
| ATOM | 19613 | CB | ILE | I | 241 | 15.277 | 44.923 | 51.765 | 1.00 | 40.13 | C |
| ATOM | 19615 | CG1 | ILE | I | 241 | 15.394 | 46.236 | 51.032 | 1.00 | 38.79 | C |
| ATOM | 19618 | CD1 | ILE | I | 241 | 16.647 | 46.930 | 51.577 | 1.00 | 69.59 | C |
| ATOM | 19622 | CG2 | ILE | I | 241 | 16.651 | 44.627 | 52.469 | 1.00 | 36.97 | C |
| ATOM | 19626 | C | ILE | I | 241 | 14.541 | 44.232 | 49.387 | 1.00 | 40.07 | C |
| ATOM | 19627 | O | ILE | I | 241 | 15.450 | 44.557 | 48.621 | 1.00 | 34.12 | O |
| ATOM | 19628 | N | ALA | I | 242 | 13.258 | 44.123 | 49.051 | 1.00 | 42.99 | N |
| ATOM | 19630 | CA | ALA | I | 242 | 12.697 | 44.379 | 47.738 | 1.00 | 44.01 | C |
| ATOM | 19632 | CB | ALA | I | 242 | 12.571 | 45.830 | 47.501 | 1.00 | 46.03 | C |
| ATOM | 19636 | C | ALA | I | 242 | 13.582 | 43.708 | 46.737 | 1.00 | 50.13 | C |
| ATOM | 19637 | O | ALA | I | 242 | 14.023 | 42.600 | 47.015 | 1.00 | 53.33 | O |
| ATOM | 19638 | N | ASP | I | 243 | 13.823 | 44.319 | 45.582 | 1.00 | 55.00 | N |
| ATOM | 19640 | CA | ASP | I | 243 | 14.728 | 43.689 | 44.632 | 1.00 | 59.22 | C |
| ATOM | 19642 | CB | ASP | I | 243 | 14.184 | 43.737 | 43.225 | 1.00 | 68.93 | C |
| ATOM | 19645 | CG | ASP | I | 243 | 14.437 | 42.445 | 42.520 | 1.00 | 66.03 | C |
| ATOM | 19646 | OD1 | ASP | I | 243 | 15.067 | 42.446 | 41.443 | 1.00 | 85.81 | O |
| ATOM | 19647 | OD2 | ASP | I | 243 | 14.095 | 41.384 | 43.071 | 1.00 | 56.12 | O |
| ATOM | 19648 | C | ASP | I | 243 | 16.145 | 44.180 | 44.611 | 1.00 | 52.80 | C |
| ATOM | 19649 | O | ASP | I | 243 | 16.741 | 44.360 | 43.548 | 1.00 | 60.45 | O |
| ATOM | 19650 | N | MET | I | 244 | 16.636 | 44.488 | 45.799 | 1.00 | 43.78 | N |
| ATOM | 19652 | CA | MET | I | 244 | 17.498 | 45.648 | 45.940 | 1.00 | 45.07 | C |
| ATOM | 19654 | CB | MET | I | 244 | 16.774 | 46.750 | 46.740 | 1.00 | 38.52 | C |
| ATOM | 19657 | CG | MET | I | 244 | 17.587 | 48.025 | 46.758 | 1.00 | 50.33 | C |
| ATOM | 19660 | SD | MET | I | 244 | 16.730 | 49.547 | 47.152 | 1.00 | 49.28 | S |
| ATOM | 19661 | CE | MET | I | 244 | 15.712 | 48.870 | 48.158 | 1.00 | 38.19 | C |
| ATOM | 19665 | C | MET | I | 244 | 18.763 | 45.143 | 46.646 | 1.00 | 44.35 | C |
| ATOM | 19666 | O | MET | I | 244 | 19.865 | 45.725 | 46.581 | 1.00 | 39.89 | O |
| ATOM | 19667 | N | GLY | I | 245 | 18.582 | 44.035 | 47.352 | 1.00 | 35.72 | N |
| ATOM | 19669 | CA | GLY | I | 245 | 19.567 | 43.670 | 48.338 | 1.00 | 39.28 | C |
| ATOM | 19672 | C | GLY | I | 245 | 18.968 | 43.053 | 49.574 | 1.00 | 39.40 | C |
| ATOM | 19673 | O | GLY | I | 245 | 17.811 | 42.647 | 49.582 | 1.00 | 43.93 | O |
| ATOM | 19674 | N | HIS | I | 246 | 19.769 | 43.023 | 50.628 | 1.00 | 41.71 | N |
| ATOM | 19676 | CA | HIS | I | 246 | 19.379 | 42.300 | 51.817 | 1.00 | 46.79 | C |
| ATOM | 19678 | CB | HIS | I | 246 | 19.886 | 40.884 | 51.670 | 1.00 | 45.02 | C |
| ATOM | 19681 | CG | HIS | I | 246 | 21.207 | 40.804 | 50.979 | 1.00 | 60.55 | C |
| ATOM | 19682 | ND1 | HIS | I | 246 | 21.341 | 40.306 | 49.697 | 1.00 | 75.57 | N |
| ATOM | 19684 | CE1 | HIS | I | 246 | 22.616 | 40.336 | 49.356 | 1.00 | 89.85 | C |
| ATOM | 19686 | NE2 | HIS | I | 246 | 23.302 | 40.872 | 50.354 | 1.00 | 89.38 | N |
| ATOM | 19688 | CD2 | HIS | I | 246 | 22.443 | 41.189 | 51.379 | 1.00 | 53.45 | C |
| ATOM | 19690 | C | HIS | I | 246 | 19.923 | 42.923 | 53.106 | 1.00 | 45.12 | C |
| ATOM | 19691 | O | HIS | I | 246 | 20.793 | 43.780 | 53.100 | 1.00 | 45.49 | O |
| ATOM | 19692 | N | LEU | I | 247 | 19.409 | 42.450 | 54.227 | 1.00 | 44.22 | N |
| ATOM | 19694 | CA | LEU | I | 247 | 20.000 | 42.752 | 55.508 | 1.00 | 41.16 | C |
| ATOM | 19696 | CB | LEU | I | 247 | 18.952 | 43.421 | 66.383 | 1.00 | 43.21 | C |
| ATOM | 19699 | CG | LEU | I | 247 | 19.266 | 44.478 | 57.428 | 1.00 | 39.08 | C |
| ATOM | 19701 | CD1 | LEU | I | 247 | 20.564 | 45.230 | 57.121 | 1.00 | 43.65 | C |
| ATOM | 19706 | CD2 | LEU | I | 247 | 18.077 | 45.408 | 57.373 | 1.00 | 49.22 | C |
| ATOM | 19709 | C | LEU | I | 247 | 20.302 | 41.402 | 56.071 | 1.00 | 39.87 | C |
| ATOM | 19710 | O | LEU | I | 247 | 19.373 | 40.633 | 56.290 | 1.00 | 39.40 | O |
| ATOM | 19711 | N | LYS | I | 248 | 21.585 | 41.103 | 56.266 | 1.00 | 47.55 | N |
| ATOM | 19713 | CA | LYS | I | 248 | 22.004 | 39.875 | 56.968 | 1.00 | 47.07 | C |
| ATOM | 19715 | CB | LYS | I | 248 | 23.084 | 39.184 | 56.159 | 1.00 | 41.75 | C |
| ATOM | 19718 | CG | LYS | I | 248 | 22.684 | 38.921 | 54.714 | 1.00 | 52.85 | C |
| ATOM | 19721 | CD | LYS | I | 248 | 23.226 | 37.572 | 54.213 | 1.00 | 60.18 | C |
| ATOM | 19724 | CE | LYS | I | 248 | 23.815 | 37.667 | 52.822 | 1.00 | 56.40 | C |
| ATOM | 19727 | NZ | LYS | I | 248 | 23.422 | 36.444 | 52.118 | 1.00 | 65.42 | N |
| ATOM | 19731 | C | LYS | I | 248 | 22.493 | 40.072 | 58.417 | 1.00 | 42.34 | C |
| ATOM | 19732 | O | LYS | I | 248 | 23.295 | 40.962 | 58.677 | 1.00 | 42.50 | O |
| ATOM | 19733 | N | TYR | I | 249 | 21.967 | 39.294 | 59.367 | 1.00 | 38.55 | N |
| ATOM | 19735 | CA | TYR | I | 249 | 22.481 | 39.349 | 60.741 | 1.00 | 44.83 | C |
| ATOM | 19737 | CB | TYR | I | 249 | 21.347 | 39.488 | 61.773 | 1.00 | 41.64 | C |
| ATOM | 19740 | CG | TYR | I | 249 | 20.502 | 40.751 | 61.596 | 1.00 | 40.56 | C |
| ATOM | 19741 | CD1 | TYR | I | 249 | 20.795 | 41.930 | 62.246 | 1.00 | 34.70 | C |
| ATOM | 19743 | CE1 | TYR | I | 249 | 20.008 | 43.051 | 62.076 | 1.00 | 43.29 | C |
| ATOM | 19745 | CZ | TYR | I | 249 | 18.926 | 43.024 | 61.227 | 1.00 | 45.55 | C |
| ATOM | 19746 | OH | TYR | I | 249 | 18.110 | 44.117 | 60.994 | 1.00 | 36.51 | O |
| ATOM | 19748 | CE2 | TYR | I | 249 | 18.654 | 41.874 | 60.556 | 1.00 | 55.36 | C |
| ATOM | 19750 | CD2 | TYR | I | 249 | 19.426 | 40.759 | 60.745 | 1.00 | 33.79 | C |
| ATOM | 19752 | C | TYR | I | 249 | 23.327 | 38.102 | 61.014 | 1.00 | 47.58 | C |
| ATOM | 19753 | O | TYR | I | 249 | 22.882 | 37.001 | 60.693 | 1.00 | 47.52 | O |
| ATOM | 19754 | N | TYR | I | 250 | 24.559 | 38.276 | 61.507 | 1.00 | 42.42 | N |
| ATOM | 19756 | CA | TYR | I | 250 | 25.407 | 37.141 | 61.882 | 1.00 | 40.79 | C |
| ATOM | 19758 | CB | TYR | I | 250 | 26.834 | 37.265 | 61.322 | 1.00 | 39.16 | C |
| ATOM | 19761 | CG | TYR | I | 250 | 26.877 | 37.276 | 59.815 | 1.00 | 52.20 | C |
| ATOM | 19762 | CD1 | TYR | I | 250 | 26.625 | 38.445 | 59.106 | 1.00 | 33.93 | C |
| ATOM | 19764 | CE1 | TYR | I | 250 | 26.621 | 38.445 | 57.717 | 1.00 | 49.30 | C |
| ATOM | 19766 | CZ | TYR | I | 250 | 26.834 | 37.283 | 57.012 | 1.00 | 39.92 | C |
| ATOM | 19767 | OH | TYR | I | 250 | 26.833 | 37.347 | 55.635 | 1.00 | 49.60 | O |
| ATOM | 19769 | CE2 | TYR | I | 250 | 27.081 | 36.109 | 57.687 | 1.00 | 37.12 | C |
| ATOM | 19771 | CD2 | TYR | I | 250 | 27.122 | 36.108 | 59.089 | 1.00 | 51.03 | C |
| ATOM | 19773 | C | TYR | I | 250 | 25.493 | 37.126 | 63.388 | 1.00 | 43.96 | C |
| ATOM | 19774 | O | TYR | I | 250 | 25.765 | 38.178 | 63.996 | 1.00 | 42.62 | O |
| ATOM | 19775 | N | LEU | I | 251 | 25.255 | 35.943 | 63.963 | 1.00 | 41.41 | N |
| ATOM | 19777 | CA | LEU | I | 251 | 25.282 | 35.747 | 65.413 | 1.00 | 41.27 | C |
| ATOM | 19779 | CB | LEU | I | 251 | 23.882 | 35.454 | 65.936 | 1.00 | 37.91 | C |
| ATOM | 19782 | CG | LEU | I | 251 | 23.770 | 34.994 | 67.387 | 1.00 | 43.71 | C |
| ATOM | 19784 | CD1 | LEU | I | 251 | 24.247 | 36.102 | 68.328 | 1.00 | 34.99 | C |
| ATOM | 19788 | CD2 | LEU | I | 251 | 22.306 | 34.598 | 67.707 | 1.00 | 41.78 | C |
| ATOM | 19792 | C | LEU | I | 251 | 26.216 | 34.621 | 65.803 | 1.00 | 45.52 | C |
| ATOM | 19793 | O | LEU | I | 251 | 26.013 | 33.460 | 65.460 | 1.00 | 48.77 | O |
| ATOM | 19794 | N | ALA | I | 252 | 27.269 | 34.966 | 66.524 | 1.00 | 51.08 | N |
| ATOM | 19796 | CA | ALA | I | 252 | 28.156 | 33.944 | 67.055 | 1.00 | 50.11 | C |
| ATOM | 19798 | CB | ALA | I | 252 | 29.290 | 34.625 | 67.776 | 1.00 | 55.86 | C |
| ATOM | 19802 | C | ALA | I | 252 | 27.439 | 32.957 | 67.996 | 1.00 | 42.93 | C |
| ATOM | 19803 | O | ALA | I | 252 | 26.732 | 33.385 | 68.895 | 1.00 | 30.97 | O |
| ATOM | 19804 | N | PRO | I | 253 | 27.683 | 31.656 | 67.812 | 1.00 | 42.71 | N |
| ATOM | 19805 | CA | PRO | I | 253 | 27.062 | 30.577 | 68.599 | 1.00 | 43.64 | C |
| ATOM | 19807 | CB | PRO | I | 253 | 27.571 | 29.294 | 67.951 | 1.00 | 32.54 | C |
| ATOM | 19810 | CG | PRO | I | 253 | 28.733 | 29.661 | 67.177 | 1.00 | 47.97 | C |
| ATOM | 19813 | CD | PRO | I | 253 | 28.594 | 31.117 | 66.789 | 1.00 | 46.33 | C |
| ATOM | 19816 | C | PRO | I | 253 | 27.445 | 30.551 | 70.050 | 1.00 | 43.85 | C |
| ATOM | 19817 | O | PRO | I | 253 | 28.292 | 31.347 | 70.422 | 1.00 | 55.58 | O |
| ATOM | 19818 | N | LYS | I | 254 | 26.779 | 29.717 | 70.846 | 1.00 | 59.79 | N |
| ATOM | 19820 | CA | LYS | I | 254 | 27.196 | 29.364 | 72.218 | 1.00 | 61.18 | C |
| ATOM | 19822 | CB | LYS | I | 254 | 26.013 | 29.405 | 73.183 | 1.00 | 56.43 | C |
| ATOM | 19825 | CG | LYS | I | 254 | 25.613 | 30.767 | 73.704 | 1.00 | 68.26 | C |
| ATOM | 19828 | CD | LYS | I | 254 | 24.518 | 30.598 | 74.743 | 1.00 | 61.63 | C |
| ATOM | 19831 | CE | LYS | I | 254 | 24.874 | 31.269 | 76.034 | 1.00 | 61.21 | C |
| ATOM | 19834 | NZ | LYS | I | 254 | 24.607 | 32.726 | 75.854 | 1.00 | 90.96 | N |
| ATOM | 19838 | C | LYS | I | 254 | 27.666 | 27.915 | 72.229 | 1.00 | 63.73 | C |
| ATOM | 19839 | O | LYS | I | 254 | 26.850 | 26.998 | 72.219 | 1.00 | 61.35 | O |
| ATOM | 19840 | N | ILE | I | 255 | 28.977 | 27.709 | 72.240 | 1.00 | 75.68 | N |
| ATOM | 19842 | CA | ILE | I | 255 | 29.553 | 26.374 | 72.098 | 1.00 | 78.83 | C |
| ATOM | 19844 | CB | ILE | I | 255 | 30.645 | 26.370 | 70.956 | 1.00 | 77.15 | C |
| ATOM | 19846 | CG1 | ILE | I | 255 | 30.710 | 25.001 | 70.240 | 1.00 | 84.09 | C |
| ATOM | 19849 | CD1 | ILE | I | 255 | 29.591 | 23.951 | 70.577 | 1.00 | 86.11 | C |
| ATOM | 19853 | CG2 | ILE | I | 255 | 32.036 | 26.859 | 71.467 | 1.00 | 66.53 | C |
| ATOM | 19857 | C | ILE | I | 255 | 30.099 | 25.995 | 73.470 | 1.00 | 83.02 | C |
| ATOM | 19858 | O | ILE | I | 255 | 30.679 | 26.840 | 74.142 | 1.00 | 79.20 | O |
| ATOM | 19859 | N | GLU | I | 256 | 29.859 | 24.760 | 73.907 | 1.00 | 96.57 | N |
| ATOM | 19861 | CA | GLU | I | 256 | 30.870 | 23.994 | 74.655 | 1.00 | 106.62 | C |
| ATOM | 19863 | CB | GLU | I | 256 | 30.303 | 22.637 | 75.100 | 1.00 | 106.20 | C |
| ATOM | 19866 | CG | GLU | I | 256 | 30.115 | 22.499 | 76.604 | 1.00 | 112.93 | C |
| ATOM | 19869 | CD | GLU | I | 256 | 28.915 | 21.637 | 76.970 | 1.00 | 123.31 | C |
| ATOM | 19870 | OE1 | GLU | I | 256 | 28.713 | 21.386 | 78.183 | 1.00 | 116.47 | O |
| ATOM | 19871 | OE2 | GLU | I | 256 | 28.177 | 21.209 | 76.050 | 1.00 | 119.68 | O |
| ATOM | 19872 | C | GLU | I | 256 | 32.158 | 23.780 | 73.330 | 1.00 | 110.18 | C |
| ATOM | 19873 | O | GLU | I | 256 | 32.177 | 22.981 | 72.889 | 1.00 | 112.91 | O |
| ATOM | 19874 | N | ASP | I | 257 | 33.209 | 24.539 | 74.139 | 1.00 | 112.54 | N |
| ATOM | 19876 | CA | ASP | I | 257 | 34.556 | 23.998 | 74.344 | 1.00 | 114.98 | C |
| ATOM | 19878 | CB | ASP | I | 257 | 34.575 | 23.069 | 75.569 | 1.00 | 114.96 | C |
| ATOM | 19881 | CG | ASP | I | 257 | 34.652 | 23.843 | 76.881 | 1.00 | 105.89 | C |
| ATOM | 19882 | OD1 | ASP | I | 257 | 33.633 | 23.966 | 77.592 | 1.00 | 75.11 | O |
| ATOM | 19883 | OD2 | ASP | I | 257 | 35.690 | 24.417 | 77.259 | 1.00 | 95.96 | O |
| ATOM | 19884 | C | ASP | I | 257 | 35.142 | 23.300 | 73.110 | 1.00 | 118.24 | C |
| ATOM | 19885 | O | ASP | I | 257 | 35.832 | 23.918 | 72.293 | 1.00 | 118.11 | O |
| ATOM | 19886 | N | MET | K | 1 | −26.686 | 81.756 | 90.463 | 1.00 | 66.60 | N |
| ATOM | 19888 | CA | MET | K | 1 | −26.431 | 80.349 | 89.996 | 1.00 | 68.69 | C |
| ATOM | 19890 | CB | MET | K | 1 | −27.737 | 79.552 | 90.013 | 1.00 | 70.09 | C |
| ATOM | 19893 | CG | MET | K | 1 | −27.610 | 78.030 | 89.972 | 1.00 | 63.53 | C |
| ATOM | 19896 | SD | MET | K | 1 | −26.850 | 77.329 | 91.485 | 1.00 | 92.36 | S |
| ATOM | 19897 | CE | MET | K | 1 | −27.540 | 75.643 | 91.429 | 1.00 | 100.99 | C |
| ATOM | 19901 | C | MET | K | 1 | −25.865 | 80.289 | 88.578 | 1.00 | 68.45 | C |
| ATOM | 19902 | O | MET | K | 1 | −26.527 | 80.766 | 87.648 | 1.00 | 70.92 | O |
| ATOM | 19905 | N | PHE | K | 2 | −24.696 | 79.661 | 88.412 | 1.00 | 60.37 | N |
| ATOM | 19907 | CA | PHE | K | 2 | −24.182 | 79.304 | 87.080 | 1.00 | 59.76 | C |
| ATOM | 19909 | CB | PHE | K | 2 | −22.679 | 79.626 | 86.968 | 1.00 | 63.22 | C |
| ATOM | 19912 | CG | PHE | K | 2 | −21.987 | 79.015 | 85.768 | 1.00 | 41.66 | C |
| ATOM | 19913 | CD1 | PHE | K | 2 | −22.311 | 79.403 | 84.486 | 1.00 | 55.02 | C |
| ATOM | 19915 | CE1 | PHE | K | 2 | −21.668 | 78.837 | 83.389 | 1.00 | 67.49 | C |
| ATOM | 19917 | CZ | PHE | K | 2 | −20.674 | 77.877 | 83.575 | 1.00 | 56.23 | C |
| ATOM | 19919 | CE2 | PHE | K | 2 | −20.345 | 77.488 | 84.846 | 1.00 | 51.50 | C |
| ATOM | 19921 | CD2 | PHE | K | 2 | −20.990 | 78.065 | 85.936 | 1.00 | 58.92 | C |
| ATOM | 19923 | C | PHE | K | 2 | −24.422 | 77.835 | 86.704 | 1.00 | 61.29 | C |
| ATOM | 19924 | O | PHE | K | 2 | −24.195 | 76.909 | 87.501 | 1.00 | 55.62 | O |
| ATOM | 19925 | N | GLU | K | 3 | −24.846 | 77.628 | 85.461 | 1.00 | 57.42 | N |
| ATOM | 19927 | CA | GLU | K | 3 | −25.191 | 76.287 | 85.013 | 1.00 | 60.75 | C |
| ATOM | 19929 | CB | GLU | K | 3 | −26.556 | 75.894 | 85.585 | 1.00 | 66.26 | C |
| ATOM | 19932 | CG | GLU | K | 3 | −27.197 | 74.596 | 85.096 | 1.00 | 78.78 | C |
| ATOM | 19935 | CD | GLU | K | 3 | −28.215 | 74.073 | 86.109 | 1.00 | 92.17 | C |
| ATOM | 19936 | OE1 | GLU | K | 3 | −28.101 | 72.885 | 86.483 | 1.00 | 84.18 | O |
| ATOM | 19937 | OE2 | GLU | K | 3 | −29.096 | 74.854 | 86.568 | 1.00 | 75.68 | O |
| ATOM | 19938 | C | GLU | K | 3 | −25.153 | 76.247 | 83.496 | 1.00 | 53.61 | C |
| ATOM | 19939 | O | GLU | K | 3 | −25.786 | 77.032 | 82.811 | 1.00 | 56.67 | O |
| ATOM | 19940 | N | ALA | K | 4 | −24.290 | 75.400 | 82.973 | 1.00 | 53.97 | N |
| ATOM | 19942 | CA | ALA | K | 4 | −24.162 | 75.288 | 81.538 | 1.00 | 57.18 | C |
| ATOM | 19944 | CB | ALA | K | 4 | −22.885 | 75.955 | 81.094 | 1.00 | 56.93 | C |
| ATOM | 19948 | C | ALA | K | 4 | −24.208 | 73.811 | 81.122 | 1.00 | 55.95 | C |
| ATOM | 19949 | O | ALA | K | 4 | −23.577 | 72.967 | 81.764 | 1.00 | 51.74 | O |
| ATOM | 19950 | N | ARG | K | 5 | −25.020 | 73.503 | 80.109 | 1.00 | 48.29 | N |
| ATOM | 19952 | CA | ARG | K | 5 | −25.105 | 72.150 | 79.570 | 1.00 | 55.41 | C |
| ATOM | 19954 | CB | ARG | K | 5 | −26.562 | 71.658 | 79.667 | 1.00 | 60.06 | C |
| ATOM | 19957 | CG | ARG | K | 5 | −26.879 | 70.282 | 79.057 | 1.00 | 60.52 | C |
| ATOM | 19960 | CD | ARG | K | 5 | −28.362 | 69.853 | 79.156 | 1.00 | 59.42 | C |
| ATOM | 19963 | NE | ARG | K | 5 | −28.597 | 69.041 | 80.350 | 1.00 | 70.15 | N |
| ATOM | 19965 | CZ | ARG | K | 5 | −29.759 | 68.904 | 80.968 | 1.00 | 74.08 | C |
| ATOM | 19966 | NH1 | ARG | K | 5 | −30.841 | 69.532 | 80.516 | 1.00 | 91.28 | N |
| ATOM | 19969 | NH2 | ARG | K | 5 | −29.833 | 68.131 | 82.042 | 1.00 | 71.59 | N |
| ATOM | 19972 | C | ARG | K | 5 | −24.547 | 72.090 | 78.133 | 1.00 | 54.31 | C |
| ATOM | 19973 | O | ARG | K | 5 | −24.775 | 72.972 | 77.312 | 1.00 | 56.71 | O |
| ATOM | 19974 | N | LEU | K | 6 | −23.733 | 71.082 | 77.867 | 1.00 | 56.78 | N |
| ATOM | 19976 | CA | LEU | K | 6 | −23.253 | 70.774 | 76.522 | 1.00 | 57.90 | C |
| ATOM | 19978 | CB | LEU | K | 6 | −21.756 | 71.067 | 76.493 | 1.00 | 61.55 | C |
| ATOM | 19981 | CG | LEU | K | 6 | −21.016 | 71.429 | 75.205 | 1.00 | 67.00 | C |
| ATOM | 19983 | CD1 | LEU | K | 6 | −21.501 | 72.753 | 74.646 | 1.00 | 76.81 | C |
| ATOM | 19987 | CD2 | LEU | K | 6 | −19.545 | 71.564 | 75.560 | 1.00 | 62.74 | C |
| ATOM | 19991 | C | LEU | K | 6 | −23.474 | 69.271 | 76.275 | 1.00 | 61.72 | C |
| ATOM | 19992 | O | LEU | K | 6 | −23.006 | 68.403 | 77.043 | 1.00 | 57.50 | O |
| ATOM | 19993 | N | VAL | K | 7 | −24.275 | 68.953 | 75.266 | 1.00 | 59.72 | N |
| ATOM | 19995 | CA | VAL | K | 7 | −24.583 | 67.547 | 75.012 | 1.00 | 62.33 | C |
| ATOM | 19997 | CB | VAL | K | 7 | −25.900 | 67.315 | 74.207 | 1.00 | 60.14 | C |
| ATOM | 19999 | CG1 | VAL | K | 7 | −27.032 | 68.170 | 74.755 | 1.00 | 51.27 | C |
| ATOM | 20003 | CG2 | VAL | K | 7 | −25.682 | 67.507 | 72.711 | 1.00 | 56.97 | C |
| ATOM | 20007 | C | VAL | K | 7 | −23.446 | 66.879 | 74.258 | 1.00 | 60.72 | C |
| ATOM | 20008 | O | VAL | K | 7 | −23.292 | 65.652 | 74.315 | 1.00 | 66.37 | O |
| ATOM | 20009 | N | GLN | K | 8 | −22.705 | 67.703 | 73.527 | 1.00 | 55.49 | N |
| ATOM | 20011 | CA | GLN | K | 8 | −21.380 | 67.374 | 73.068 | 1.00 | 58.65 | C |
| ATOM | 20013 | CB | GLN | K | 8 | −21.042 | 68.275 | 71.893 | 1.00 | 62.60 | C |
| ATOM | 20016 | CG | GLN | K | 8 | −21.112 | 67.542 | 70.573 | 1.00 | 77.97 | C |
| ATOM | 20019 | CD | GLN | K | 8 | −19.827 | 67.659 | 69.799 | 1.00 | 75.24 | C |
| ATOM | 20020 | OE1 | GLN | K | 8 | −18.970 | 66.784 | 69.878 | 1.00 | 90.43 | O |
| ATOM | 20021 | NE2 | GLN | K | 8 | −19.672 | 68.756 | 69.081 | 1.00 | 59.39 | N |
| ATOM | 20024 | C | GLN | K | 8 | −20.328 | 67.546 | 74.151 | 1.00 | 61.88 | C |
| ATOM | 20025 | O | GLN | K | 8 | −19.453 | 68.408 | 74.067 | 1.00 | 65.53 | O |
| ATOM | 20026 | N | GLY | K | 9 | −20.362 | 66.675 | 75.148 | 1.00 | 63.76 | N |
| ATOM | 20028 | CA | GLY | K | 9 | −19.408 | 66.783 | 76.235 | 1.00 | 63.30 | C |
| ATOM | 20031 | C | GLY | K | 9 | −17.995 | 66.739 | 75.707 | 1.00 | 56.48 | C |
| ATOM | 20032 | O | GLY | K | 9 | −17.075 | 67.331 | 76.256 | 1.00 | 53.28 | O |
| ATOM | 20033 | N | SER | K | 10 | −17.841 | 65.982 | 74.635 | 1.00 | 58.17 | N |
| ATOM | 20035 | CA | SER | K | 10 | −16.568 | 65.891 | 73.937 | 1.00 | 59.81 | C |
| ATOM | 20037 | CB | SER | K | 10 | −16.783 | 65.422 | 72.494 | 1.00 | 61.64 | C |
| ATOM | 20040 | OG | SER | K | 10 | −16.507 | 66.483 | 71.576 | 1.00 | 76.95 | O |
| ATOM | 20042 | C | SER | K | 10 | −15.780 | 67.211 | 73.960 | 1.00 | 49.61 | C |
| ATOM | 20043 | O | SER | K | 10 | −14.588 | 67.194 | 74.204 | 1.00 | 39.84 | O |
| ATOM | 20044 | N | ILE | K | 11 | −16.415 | 68.337 | 73.661 | 1.00 | 50.75 | N |
| ATOM | 20046 | CA | ILE | K | 11 | −15.691 | 69.585 | 73.410 | 1.00 | 50.68 | C |
| ATOM | 20048 | CB | ILE | K | 11 | −16.695 | 70.719 | 73.068 | 1.00 | 50.44 | C |
| ATOM | 20050 | CG1 | ILE | K | 11 | −17.028 | 70.724 | 71.574 | 1.00 | 60.77 | C |
| ATOM | 20053 | CD1 | ILE | K | 11 | −18.432 | 70.273 | 71.230 | 1.00 | 56.60 | C |
| ATOM | 20057 | CG2 | ILE | K | 11 | −16.133 | 72.089 | 73.455 | 1.00 | 49.69 | C |
| ATOM | 20061 | C | ILE | K | 11 | −14.894 | 69.973 | 74.665 | 1.00 | 53.12 | C |
| ATOM | 20062 | O | ILE | K | 11 | −13.690 | 70.261 | 74.635 | 1.00 | 50.42 | O |
| ATOM | 20063 | N | LEU | K | 12 | −15.581 | 69.956 | 75.795 | 1.00 | 50.94 | N |
| ATOM | 20065 | CA | LEU | K | 12 | −14.945 | 70.317 | 77.050 | 1.00 | 53.45 | C |
| ATOM | 20067 | CB | LEU | K | 12 | −16.019 | 70.376 | 78.148 | 1.00 | 58.03 | C |
| ATOM | 20070 | CG | LEU | K | 12 | −16.203 | 71.711 | 78.871 | 1.00 | 49.15 | C |
| ATOM | 20072 | CD1 | LEU | K | 12 | −17.165 | 72.563 | 78.117 | 1.00 | 61.06 | C |
| ATOM | 20076 | CD2 | LEU | K | 12 | −16.760 | 71.477 | 80.258 | 1.00 | 79.73 | C |
| ATOM | 20080 | C | LEU | K | 12 | −13.807 | 69.359 | 77.465 | 1.00 | 46.34 | C |
| ATOM | 20081 | O | LEU | K | 12 | −12.789 | 69.773 | 78.003 | 1.00 | 44.90 | O |
| ATOM | 20082 | N | LYS | K | 13 | −14.029 | 68.063 | 77.321 | 1.00 | 46.04 | N |
| ATOM | 20084 | CA | LYS | K | 13 | −12.972 | 67.070 | 77.450 | 1.00 | 40.94 | C |
| ATOM | 20086 | CB | LYS | K | 13 | −13.554 | 65.737 | 76.992 | 1.00 | 30.53 | C |
| ATOM | 20089 | CG | LYS | K | 13 | −14.083 | 64.854 | 78.109 | 1.00 | 43.07 | C |
| ATOM | 20092 | CD | LYS | K | 13 | −14.546 | 63.465 | 77.565 | 1.00 | 34.38 | C |
| ATOM | 20095 | CE | LYS | K | 13 | −13.550 | 62.330 | 77.661 | 1.00 | 41.73 | C |
| ATOM | 20098 | NZ | LYS | K | 13 | −12.407 | 62.501 | 78.624 | 1.00 | 60.40 | N |
| ATOM | 20102 | C | LYS | K | 13 | −11.690 | 67.424 | 76.644 | 1.00 | 42.81 | C |
| ATOM | 20103 | O | LYS | K | 13 | −10.600 | 67.509 | 77.208 | 1.00 | 40.78 | O |
| ATOM | 20104 | N | LYS | K | 14 | −11.819 | 67.730 | 75.357 | 1.00 | 39.19 | N |
| ATOM | 20106 | CA | LYS | K | 14 | −10.713 | 68.268 | 74.572 | 1.00 | 41.47 | C |
| ATOM | 20108 | CB | LYS | K | 14 | −11.164 | 68.410 | 73.116 | 1.00 | 45.10 | C |
| ATOM | 20111 | CG | LYS | K | 14 | −11.510 | 67.039 | 72.506 | 1.00 | 44.49 | C |
| ATOM | 20114 | CD | LYS | K | 14 | −12.309 | 67.123 | 71.234 | 1.00 | 46.21 | C |
| ATOM | 20117 | CE | LYS | K | 14 | −12.164 | 65.844 | 70.438 | 1.00 | 50.33 | C |
| ATOM | 20120 | NZ | LYS | K | 14 | −13.260 | 65.734 | 69.463 | 1.00 | 63.29 | N |
| ATOM | 20124 | C | LYS | K | 14 | −10.058 | 69.559 | 75.076 | 1.00 | 42.64 | C |
| ATOM | 20125 | O | LYS | K | 14 | −8.816 | 69.680 | 75.127 | 1.00 | 35.96 | O |
| ATOM | 20126 | N | VAL | K | 15 | −10.902 | 70.529 | 75.420 | 1.00 | 43.91 | N |
| ATOM | 20128 | CA | VAL | K | 15 | −10.428 | 71.825 | 75.876 | 1.00 | 42.75 | C |
| ATOM | 20130 | CB | VAL | K | 15 | −11.606 | 72.768 | 76.223 | 1.00 | 38.12 | C |
| ATOM | 20132 | CG1 | VAL | K | 15 | −11.139 | 74.083 | 76.816 | 1.00 | 46.04 | C |
| ATOM | 20136 | CG2 | VAL | K | 15 | −12.355 | 73.126 | 74.978 | 1.00 | 55.18 | C |
| ATOM | 20140 | C | VAL | K | 15 | −9.518 | 71.548 | 77.062 | 1.00 | 40.95 | C |
| ATOM | 20141 | O | VAL | K | 15 | −8.374 | 72.003 | 77.172 | 1.00 | 45.32 | O |
| ATOM | 20142 | N | LEU | K | 16 | −10.027 | 70.755 | 77.972 | 1.00 | 44.07 | N |
| ATOM | 20144 | CA | LEU | K | 16 | −9.274 | 70.570 | 79.196 | 1.00 | 54.89 | C |
| ATOM | 20146 | CB | LEU | K | 16 | −10.168 | 70.066 | 80.343 | 1.00 | 53.62 | C |
| ATOM | 20149 | CG | LEU | K | 16 | −10.402 | 71.284 | 81.242 | 1.00 | 60.90 | C |
| ATOM | 20151 | CD1 | LEU | K | 16 | −11.859 | 71.590 | 81.547 | 1.00 | 56.67 | C |
| ATOM | 20155 | CD2 | LEU | K | 16 | −9.567 | 71.151 | 82.496 | 1.00 | 67.37 | C |
| ATOM | 20159 | C | LEU | K | 16 | −7.984 | 69.768 | 79.016 | 1.00 | 47.36 | C |
| ATOM | 20160 | O | LEU | K | 16 | −7.024 | 70.025 | 79.717 | 1.00 | 48.89 | O |
| ATOM | 20161 | N | GLU | K | 17 | −7.952 | 68.845 | 78.061 | 1.00 | 43.76 | N |
| ATOM | 20163 | CA | GLU | K | 17 | −6.699 | 68.306 | 77.516 | 1.00 | 44.85 | C |
| ATOM | 20165 | CB | GLU | K | 17 | −7.004 | 67.128 | 76.584 | 1.00 | 36.20 | C |
| ATOM | 20168 | CG | GLU | K | 17 | −7.635 | 65.909 | 77.237 | 1.00 | 53.67 | C |
| ATOM | 20171 | CD | GLU | K | 17 | −6.709 | 65.258 | 78.256 | 1.00 | 87.15 | C |
| ATOM | 20172 | OE1 | GLU | K | 17 | −6.985 | 65.342 | 79.491 | 1.00 | 79.01 | O |
| ATOM | 20173 | OE2 | GLU | K | 17 | −5.689 | 64.685 | 77.794 | 1.00 | 80.08 | O |
| ATOM | 20174 | C | GLU | K | 17 | −5.794 | 69.304 | 76.758 | 1.00 | 44.73 | C |
| ATOM | 20175 | O | GLU | K | 17 | −4.571 | 69.160 | 76.713 | 1.00 | 50.98 | O |
| ATOM | 20176 | N | ALA | K | 18 | −6.368 | 70.317 | 76.130 | 1.00 | 42.79 | N |
| ATOM | 20178 | CA | ALA | K | 18 | −5.538 | 71.287 | 75.452 | 1.00 | 38.20 | C |
| ATOM | 20180 | CB | ALA | K | 18 | −6.373 | 71.968 | 74.405 | 1.00 | 43.04 | C |
| ATOM | 20184 | C | ALA | K | 18 | −4.928 | 72.317 | 76.414 | 1.00 | 40.71 | C |
| ATOM | 20185 | O | ALA | K | 18 | −4.073 | 73.113 | 76.029 | 1.00 | 48.61 | O |
| ATOM | 20186 | N | LEU | K | 19 | −5.310 | 72.276 | 77.682 | 1.00 | 41.89 | N |
| ATOM | 20188 | CA | LEU | K | 19 | −4.886 | 73.295 | 78.625 | 1.00 | 45.09 | C |
| ATOM | 20190 | CB | LEU | K | 19 | −6.105 | 73.799 | 79.391 | 1.00 | 47.89 | C |
| ATOM | 20193 | CG | LEU | K | 19 | −6.688 | 74.863 | 78.630 | 1.00 | 47.39 | C |
| ATOM | 20195 | CD1 | LEU | K | 19 | −7.942 | 75.491 | 79.502 | 1.00 | 40.79 | C |
| ATOM | 20199 | CD2 | LEU | K | 19 | −5.947 | 75.916 | 78.102 | 1.00 | 40.90 | C |
| ATOM | 20203 | C | LEU | K | 19 | −3.846 | 72.822 | 79.628 | 1.00 | 43.23 | C |
| ATOM | 20204 | O | LEU | K | 19 | −2.967 | 73.583 | 80.019 | 1.00 | 44.05 | O |
| ATOM | 20205 | N | LYS | K | 20 | −4.007 | 71.588 | 80.091 | 1.00 | 46.29 | N |
| ATOM | 20207 | CA | LYS | K | 20 | −3.482 | 71.146 | 81.387 | 1.00 | 48.28 | C |
| ATOM | 20209 | CB | LYS | K | 20 | −4.265 | 69.923 | 81.923 | 1.00 | 46.75 | C |
| ATOM | 20212 | CG | LYS | K | 20 | −4.250 | 68.689 | 81.009 | 1.00 | 40.48 | C |
| ATOM | 20215 | CD | LYS | K | 20 | −3.801 | 67.431 | 81.720 | 1.00 | 53.38 | C |
| ATOM | 20218 | CE | LYS | K | 20 | −2.863 | 66.591 | 80.856 | 1.00 | 48.00 | C |
| ATOM | 20221 | NZ | LYS | K | 20 | −3.609 | 65.828 | 79.846 | 1.00 | 57.58 | N |
| ATOM | 20225 | C | LYS | K | 20 | −1.994 | 70.806 | 81.299 | 1.00 | 48.41 | C |
| ATOM | 20226 | O | LYS | K | 20 | −1.316 | 70.004 | 82.306 | 1.00 | 59.42 | O |
| ATOM | 20227 | N | ASP | K | 21 | −1.460 | 70.522 | 80.119 | 1.00 | 49.84 | N |
| ATOM | 20229 | CA | ASP | K | 21 | −0.008 | 70.509 | 79.972 | 1.00 | 58.30 | C |
| ATOM | 20231 | CB | ASP | K | 21 | 0.439 | 69.917 | 78.635 | 1.00 | 62.39 | C |
| ATOM | 20234 | CG | ASP | K | 21 | −0.027 | 68.499 | 78.442 | 1.00 | 69.93 | C |
| ATOM | 20235 | OD1 | ASP | K | 21 | −0.316 | 67.825 | 79.459 | 1.00 | 58.32 | O |
| ATOM | 20236 | OD2 | ASP | K | 21 | −0.124 | 68.005 | 77.297 | 1.00 | 83.34 | O |
| ATOM | 20237 | C | ASP | K | 21 | 0.575 | 71.907 | 80.039 | 1.00 | 54.08 | C |
| ATOM | 20238 | O | ASP | K | 21 | 1.713 | 72.066 | 80.452 | 1.00 | 61.19 | O |
| ATOM | 20239 | N | LEU | K | 22 | −0.133 | 72.905 | 79.524 | 1.00 | 45.57 | N |
| ATOM | 20241 | CA | LEU | K | 22 | 0.491 | 74.212 | 79.434 | 1.00 | 42.79 | C |
| ATOM | 20243 | CB | LEU | K | 22 | −0.145 | 75.042 | 78.354 | 1.00 | 39.85 | C |
| ATOM | 20246 | CG | LEU | K | 22 | 0.649 | 76.315 | 78.111 | 1.00 | 47.28 | C |
| ATOM | 20248 | CD1 | LEU | K | 22 | 2.024 | 76.076 | 77.478 | 1.00 | 48.22 | C |
| ATOM | 20252 | CD2 | LEU | K | 22 | −0.199 | 77.083 | 77.173 | 1.00 | 56.85 | C |
| ATOM | 20256 | C | LEU | K | 22 | 0.403 | 74.989 | 80.732 | 1.00 | 41.45 | C |
| ATOM | 20257 | O | LEU | K | 22 | 1.273 | 75.810 | 81.045 | 1.00 | 42.84 | O |
| ATOM | 20258 | N | ILE | K | 23 | −0.685 | 74.759 | 81.455 | 1.00 | 38.64 | N |
| ATOM | 20260 | CA | ILE | K | 23 | −0.997 | 75.581 | 82.604 | 1.00 | 43.19 | C |
| ATOM | 20262 | CB | ILE | K | 23 | −1.845 | 76.807 | 82.249 | 1.00 | 38.61 | C |
| ATOM | 20264 | CG1 | ILE | K | 23 | −3.176 | 76.391 | 81.654 | 1.00 | 39.89 | C |
| ATOM | 20267 | CD1 | ILE | K | 23 | −4.086 | 77.578 | 81.481 | 1.00 | 62.78 | C |
| ATOM | 20271 | CG2 | ILE | K | 23 | −1.058 | 77.698 | 81.289 | 1.00 | 42.07 | C |
| ATOM | 20275 | C | ILE | K | 23 | −1.601 | 74.808 | 83.757 | 1.00 | 46.57 | C |
| ATOM | 20276 | O | ILE | K | 23 | −2.383 | 73.876 | 83.596 | 1.00 | 42.49 | O |
| ATOM | 20277 | N | ASN | K | 24 | −1.172 | 75.243 | 84.928 | 1.00 | 50.44 | N |
| ATOM | 20279 | CA | ASN | K | 24 | −1.183 | 74.458 | 86.138 | 1.00 | 58.92 | C |
| ATOM | 20281 | CB | ASN | K | 24 | −0.059 | 75.034 | 87.056 | 1.00 | 69.18 | C |
| ATOM | 20284 | CG | ASN | K | 24 | 0.836 | 76.155 | 86.356 | 1.00 | 75.28 | C |
| ATOM | 20285 | OD1 | ASN | K | 24 | 1.901 | 75.842 | 85.839 | 1.00 | 91.70 | O |
| ATOM | 20286 | ND2 | ASN | K | 24 | 0.431 | 77.433 | 86.400 | 1.00 | 34.73 | N |
| ATOM | 20289 | C | ASN | K | 24 | −2.619 | 74.684 | 86.673 | 1.00 | 55.74 | C |
| ATOM | 20290 | O | ASN | K | 24 | −3.410 | 73.759 | 86.820 | 1.00 | 45.08 | O |
| ATOM | 20291 | N | GLU | K | 25 | −2.952 | 75.966 | 86.835 | 1.00 | 56.97 | N |
| ATOM | 20293 | CA | GLU | K | 25 | −4.241 | 76.476 | 87.305 | 1.00 | 58.18 | C |
| ATOM | 20295 | CB | GLU | K | 25 | −4.109 | 76.983 | 88.738 | 1.00 | 63.99 | C |
| ATOM | 20298 | CG | GLU | K | 25 | −3.033 | 76.339 | 89.599 | 1.00 | 77.11 | C |
| ATOM | 20301 | CD | GLU | K | 25 | −2.040 | 77.364 | 90.068 | 1.00 | 85.15 | C |
| ATOM | 20302 | OE1 | GLU | K | 25 | −0.843 | 77.261 | 89.705 | 1.00 | 102.26 | O |
| ATOM | 20303 | OE2 | GLU | K | 25 | −2.503 | 78.301 | 90.746 | 1.00 | 87.14 | O |
| ATOM | 20304 | C | GLU | K | 25 | −4.688 | 77.695 | 86.491 | 1.00 | 54.47 | C |
| ATOM | 20305 | O | GLU | K | 25 | −3.917 | 78.296 | 85.751 | 1.00 | 64.16 | O |
| ATOM | 20306 | N | ALA | K | 26 | −5.937 | 78.092 | 86.636 | 1.00 | 49.08 | N |
| ATOM | 20308 | CA | ALA | K | 26 | −6.407 | 79.251 | 85.906 | 1.00 | 54.32 | C |
| ATOM | 20310 | CB | ALA | K | 26 | −6.492 | 78.975 | 84.408 | 1.00 | 53.99 | C |
| ATOM | 20314 | C | ALA | K | 26 | −7.751 | 79.706 | 86.420 | 1.00 | 53.29 | C |
| ATOM | 20315 | O | ALA | K | 26 | −8.395 | 79.032 | 87.212 | 1.00 | 59.52 | O |
| ATOM | 20316 | N | CYS | K | 27 | −8.180 | 80.845 | 85.909 | 1.00 | 50.60 | N |
| ATOM | 20318 | CA | CYS | K | 27 | −9.359 | 81.474 | 86.425 | 1.00 | 52.03 | C |
| ATOM | 20320 | CB | CYS | K | 27 | −9.019 | 82.868 | 86.938 | 1.00 | 57.00 | C |
| ATOM | 20323 | SG | CYS | K | 27 | −10.502 | 83.844 | 87.287 | 1.00 | 74.77 | S |
| ATOM | 20324 | C | CYS | K | 27 | −10.374 | 81.544 | 85.310 | 1.00 | 44.09 | C |
| ATOM | 20325 | O | CYS | K | 27 | −10.205 | 82.266 | 84.317 | 1.00 | 43.20 | O |
| ATOM | 20326 | N | TRP | K | 28 | −11.461 | 80.818 | 85.502 | 1.00 | 44.14 | N |
| ATOM | 20328 | CA | TRP | K | 28 | −12.580 | 80.898 | 84.569 | 1.00 | 46.37 | C |
| ATOM | 20330 | CB | TRP | K | 28 | −13.399 | 79.607 | 84.652 | 1.00 | 54.99 | C |
| ATOM | 20333 | CG | TRP | K | 28 | −12.602 | 78.383 | 84.232 | 1.00 | 56.49 | C |
| ATOM | 20334 | CD1 | TRP | K | 28 | −11.467 | 77.899 | 84.810 | 1.00 | 60.00 | C |
| ATOM | 20336 | NE1 | TRP | K | 28 | −11.041 | 76.763 | 84.163 | 1.00 | 55.57 | N |
| ATOM | 20338 | CE2 | TRP | K | 28 | −11.897 | 76.506 | 83.128 | 1.00 | 58.46 | C |
| ATOM | 20339 | CD2 | TRP | K | 28 | −12.896 | 77.503 | 83.148 | 1.00 | 54.83 | C |
| ATOM | 20340 | CE3 | TRP | K | 28 | −13.915 | 77.447 | 82.193 | 1.00 | 63.12 | C |
| ATOM | 20342 | CZ3 | TRP | K | 28 | −13.889 | 76.438 | 81.253 | 1.00 | 59.62 | C |
| ATOM | 20344 | CH2 | TRP | K | 28 | −12.899 | 75.456 | 81.283 | 1.00 | 54.62 | C |
| ATOM | 20346 | CZ2 | TRP | K | 28 | −11.893 | 75.472 | 82.205 | 1.00 | 57.24 | C |
| ATOM | 20348 | C | TRP | K | 28 | −13.455 | 82.121 | 84.819 | 1.00 | 43.36 | C |
| ATOM | 20349 | O | TRP | K | 28 | −13.946 | 82.321 | 85.918 | 1.00 | 47.42 | O |
| ATOM | 20350 | N | ASP | K | 29 | −13.558 | 82.998 | 83.827 | 1.00 | 50.69 | N |
| ATOM | 20352 | CA | ASP | K | 29 | −14.400 | 84.188 | 83.910 | 1.00 | 52.41 | C |
| ATOM | 20354 | CB | ASP | K | 29 | −13.763 | 85.385 | 83.220 | 1.00 | 52.36 | C |
| ATOM | 20357 | CG | ASP | K | 29 | −12.447 | 85.778 | 83.870 | 1.00 | 73.20 | C |
| ATOM | 20358 | OD1 | ASP | K | 29 | −12.367 | 85.643 | 85.118 | 1.00 | 72.79 | O |
| ATOM | 20359 | OD2 | ASP | K | 29 | −11.441 | 86.171 | 83.221 | 1.00 | 80.83 | O |
| ATOM | 20360 | C | ASP | K | 29 | −15.725 | 83.894 | 83.258 | 1.00 | 58.90 | C |
| ATOM | 20361 | O | ASP | K | 29 | −15.836 | 83.923 | 82.025 | 1.00 | 64.78 | O |
| ATOM | 20362 | N | ILE | K | 30 | −16.711 | 83.628 | 84.117 | 1.00 | 56.26 | N |
| ATOM | 20364 | CA | ILE | K | 30 | −18.068 | 83.339 | 83.694 | 1.00 | 54.99 | C |
| ATOM | 20366 | CB | ILE | K | 30 | −18.651 | 82.326 | 84.660 | 1.00 | 55.41 | C |
| ATOM | 20368 | CG1 | ILE | K | 30 | −17.616 | 81.231 | 84.896 | 1.00 | 63.04 | C |
| ATOM | 20371 | CD1 | ILE | K | 30 | −17.902 | 79.955 | 84.131 | 1.00 | 68.13 | C |
| ATOM | 20375 | CG2 | ILE | K | 30 | −19.979 | 81.756 | 84.122 | 1.00 | 38.91 | C |
| ATOM | 20379 | C | ILE | K | 30 | −18.949 | 84.580 | 83.662 | 1.00 | 56.39 | C |
| ATOM | 20380 | O | ILE | K | 30 | −18.981 | 85.348 | 84.615 | 1.00 | 61.33 | O |
| ATOM | 20381 | N | SER | K | 31 | −19.715 | 84.738 | 82.592 | 1.00 | 52.38 | N |
| ATOM | 20383 | CA | SER | K | 31 | −20.672 | 85.820 | 82.529 | 1.00 | 48.21 | C |
| ATOM | 20385 | CB | SER | K | 31 | −19.943 | 87.087 | 82.063 | 1.00 | 55.98 | C |
| ATOM | 20388 | OG | SER | K | 31 | −19.439 | 86.971 | 80.740 | 1.00 | 43.12 | O |
| ATOM | 20390 | C | SER | K | 31 | −21.820 | 85.459 | 81.589 | 1.00 | 49.93 | C |
| ATOM | 20391 | O | SER | K | 31 | −21.855 | 84.365 | 81.025 | 1.00 | 53.67 | O |
| ATOM | 20392 | N | SER | K | 32 | −22.752 | 86.383 | 81.373 | 1.00 | 54.92 | N |
| ATOM | 20394 | CA | SER | K | 32 | −23.847 | 86.109 | 80.445 | 1.00 | 59.30 | C |
| ATOM | 20396 | CB | SER | K | 32 | −24.899 | 87.213 | 80.436 | 1.00 | 49.97 | C |
| ATOM | 20399 | OG | SER | K | 32 | −24.597 | 88.169 | 81.431 | 1.00 | 82.71 | O |
| ATOM | 20401 | C | SER | K | 32 | −23.360 | 85.826 | 79.030 | 1.00 | 58.78 | C |
| ATOM | 20402 | O | SER | K | 32 | −23.990 | 85.059 | 78.304 | 1.00 | 66.16 | O |
| ATOM | 20403 | N | SER | K | 33 | −22.262 | 86.443 | 78.609 | 1.00 | 59.14 | N |
| ATOM | 20405 | CA | SER | K | 33 | −21.806 | 86.281 | 77.225 | 1.00 | 55.49 | C |
| ATOM | 20407 | CB | SER | K | 33 | −20.771 | 87.355 | 76.941 | 1.00 | 53.04 | C |
| ATOM | 20410 | OG | SER | K | 33 | −20.292 | 87.849 | 78.178 | 1.00 | 58.64 | O |
| ATOM | 20412 | C | SER | K | 33 | −21.215 | 84.877 | 77.013 | 1.00 | 53.53 | C |
| ATOM | 20413 | O | SER | K | 33 | −21.226 | 84.314 | 75.903 | 1.00 | 44.28 | O |
| ATOM | 20414 | N | GLY | K | 34 | −20.757 | 84.297 | 78.120 | 1.00 | 50.71 | N |
| ATOM | 20416 | CA | GLY | K | 34 | −20.240 | 82.943 | 78.121 | 1.00 | 51.07 | C |
| ATOM | 20419 | C | GLY | K | 34 | −19.022 | 82.734 | 79.005 | 1.00 | 45.43 | C |
| ATOM | 20420 | O | GLY | K | 34 | −18.758 | 83.476 | 79.973 | 1.00 | 42.62 | O |
| ATOM | 20421 | N | VAL | K | 35 | −18.280 | 81.685 | 78.680 | 1.00 | 33.85 | N |
| ATOM | 20423 | CA | VAL | K | 35 | −17.067 | 81.398 | 79.438 | 1.00 | 46.19 | C |
| ATOM | 20425 | CB | VAL | K | 35 | −16.841 | 79.880 | 79.612 | 1.00 | 51.61 | C |
| ATOM | 20427 | CG1 | VAL | K | 35 | −15.506 | 79.610 | 80.304 | 1.00 | 47.72 | C |
| ATOM | 20431 | CG2 | VAL | K | 35 | −18.023 | 79.236 | 80.366 | 1.00 | 41.80 | C |
| ATOM | 20435 | C | VAL | K | 35 | −15.840 | 82.007 | 78.796 | 1.00 | 41.97 | C |
| ATOM | 20436 | O | VAL | K | 35 | −15.740 | 82.086 | 77.586 | 1.00 | 51.50 | O |
| ATOM | 20437 | N | ASN | K | 36 | −14.899 | 82.441 | 79.619 | 1.00 | 55.34 | N |
| ATOM | 20439 | CA | ASN | K | 36 | −13.667 | 83.073 | 79.115 | 1.00 | 58.80 | C |
| ATOM | 20441 | CB | ASN | K | 36 | −13.873 | 84.572 | 78.917 | 1.00 | 58.17 | C |
| ATOM | 20444 | CG | ASN | K | 36 | −13.719 | 84.967 | 77.480 | 1.00 | 71.36 | C |
| ATOM | 20445 | OD1 | ASN | K | 36 | −12.595 | 85.137 | 77.028 | 1.00 | 96.74 | O |
| ATOM | 20446 | ND2 | ASN | K | 36 | −14.826 | 85.056 | 76.736 | 1.00 | 74.12 | N |
| ATOM | 20449 | C | ASN | K | 36 | −12.453 | 82.818 | 80.010 | 1.00 | 51.48 | C |
| ATOM | 20450 | O | ASN | K | 36 | −12.547 | 82.916 | 81.219 | 1.00 | 56.16 | O |
| ATOM | 20451 | N | LEU | K | 37 | −11.333 | 82.408 | 79.431 | 1.00 | 50.81 | N |
| ATOM | 20453 | CA | LEU | K | 37 | −10.163 | 82.028 | 80.225 | 1.00 | 46.82 | C |
| ATOM | 20455 | CB | LEU | K | 37 | −10.032 | 80.509 | 80.250 | 1.00 | 47.01 | C |
| ATOM | 20458 | CG | LEU | K | 37 | −8.928 | 80.011 | 81.171 | 1.00 | 45.93 | C |
| ATOM | 20460 | CD1 | LEU | K | 37 | −9.260 | 78.614 | 81.648 | 1.00 | 51.00 | C |
| ATOM | 20464 | CD2 | LEU | K | 37 | −7.572 | 80.046 | 80.460 | 1.00 | 41.91 | C |
| ATOM | 20468 | C | LEU | K | 37 | −8.896 | 82.642 | 79.626 | 1.00 | 42.90 | C |
| ATOM | 20469 | O | LEU | K | 37 | −8.586 | 82.403 | 78.469 | 1.00 | 48.74 | O |
| ATOM | 20470 | N | GLN | K | 38 | −8.168 | 83.452 | 80.387 | 1.00 | 39.34 | N |
| ATOM | 20472 | CA | GLN | K | 38 | −6.835 | 83.875 | 79.970 | 1.00 | 39.72 | C |
| ATOM | 20474 | CB | GLN | K | 38 | −6.757 | 85.372 | 79.877 | 1.00 | 42.27 | C |
| ATOM | 20477 | CG | GLN | K | 38 | −5.574 | 85.833 | 79.112 | 1.00 | 59.56 | C |
| ATOM | 20480 | CD | GLN | K | 38 | −5.545 | 87.340 | 79.061 | 1.00 | 68.16 | C |
| ATOM | 20481 | OE1 | GLN | K | 38 | −5.252 | 87.969 | 80.083 | 1.00 | 54.45 | O |
| ATOM | 20482 | NE2 | GLN | K | 38 | −5.868 | 87.921 | 77.892 | 1.00 | 46.85 | N |
| ATOM | 20485 | C | GLN | K | 38 | −5.820 | 83.495 | 81.019 | 1.00 | 38.05 | C |
| ATOM | 20486 | O | GLN | K | 38 | −6.079 | 83.648 | 82.193 | 1.00 | 36.68 | O |
| ATOM | 20487 | N | SER | K | 39 | −4.633 | 83.092 | 80.599 | 1.00 | 40.60 | N |
| ATOM | 20489 | CA | SER | K | 39 | −3.606 | 82.770 | 81.563 | 1.00 | 45.82 | C |
| ATOM | 20491 | CB | SER | K | 39 | −4.029 | 81.544 | 82.357 | 1.00 | 48.24 | C |
| ATOM | 20494 | OG | SER | K | 39 | −2.918 | 81.036 | 83.057 | 1.00 | 40.63 | O |
| ATOM | 20496 | C | SER | K | 39 | −2.314 | 82.486 | 80.831 | 1.00 | 47.33 | C |
| ATOM | 20497 | O | SER | K | 39 | −2.363 | 81.882 | 79.752 | 1.00 | 41.11 | O |
| ATOM | 20498 | N | MET | K | 40 | −1.184 | 82.900 | 81.413 | 1.00 | 44.07 | N |
| ATOM | 20500 | CA | MET | K | 40 | 0.114 | 82.507 | 80.867 | 1.00 | 46.93 | C |
| ATOM | 20502 | CB | MET | K | 40 | 1.175 | 83.602 | 80.955 | 1.00 | 48.81 | C |
| ATOM | 20505 | CG | MET | K | 40 | 0.792 | 84.881 | 81.594 | 1.00 | 42.33 | C |
| ATOM | 20508 | SD | MET | K | 40 | 1.859 | 86.216 | 81.057 | 1.00 | 56.22 | S |
| ATOM | 20509 | CE | MET | K | 40 | 2.170 | 87.006 | 82.582 | 1.00 | 53.39 | C |
| ATOM | 20513 | C | MET | K | 40 | 0.671 | 81.271 | 81.555 | 1.00 | 44.17 | C |
| ATOM | 20514 | O | MET | K | 40 | 0.192 | 80.892 | 82.602 | 1.00 | 47.36 | O |
| ATOM | 20515 | N | ASP | K | 41 | 1.673 | 80.628 | 80.971 | 1.00 | 41.72 | N |
| ATOM | 20517 | CA | ASP | K | 41 | 2.288 | 79.502 | 81.635 | 1.00 | 43.63 | C |
| ATOM | 20519 | CB | ASP | K | 41 | 3.025 | 78.632 | 80.628 | 1.00 | 42.81 | C |
| ATOM | 20522 | CG | ASP | K | 41 | 4.298 | 79.272 | 80.145 | 1.00 | 54.79 | C |
| ATOM | 20523 | OD1 | ASP | K | 41 | 5.417 | 78.745 | 80.398 | 1.00 | 54.50 | O |
| ATOM | 20524 | OD2 | ASP | K | 41 | 4.234 | 80.331 | 79.497 | 1.00 | 43.21 | O |
| ATOM | 20525 | C | ASP | K | 41 | 3.224 | 80.084 | 82.676 | 1.00 | 42.04 | C |
| ATOM | 20526 | O | ASP | K | 41 | 3.351 | 81.298 | 82.731 | 1.00 | 46.86 | O |
| ATOM | 20527 | N | SER | K | 42 | 3.751 | 79.230 | 83.555 | 1.00 | 48.59 | N |
| ATOM | 20529 | CA | SER | K | 42 | 4.644 | 79.613 | 84.650 | 1.00 | 52.25 | C |
| ATOM | 20531 | CB | SER | K | 42 | 5.203 | 78.367 | 85.352 | 1.00 | 52.92 | C |
| ATOM | 20534 | OG | SER | K | 42 | 6.058 | 77.597 | 84.511 | 1.00 | 69.86 | O |
| ATOM | 20536 | C | SER | K | 42 | 5.798 | 80.506 | 84.192 | 1.00 | 52.06 | C |
| ATOM | 20537 | O | SER | K | 42 | 6.170 | 81.434 | 84.881 | 1.00 | 52.43 | O |
| ATOM | 20538 | N | SER | K | 43 | 6.338 | 80.270 | 83.008 | 1.00 | 53.25 | N |
| ATOM | 20540 | CA | SER | K | 43 | 7.489 | 81.034 | 82.565 | 1.00 | 53.11 | C |
| ATOM | 20542 | CB | SER | K | 43 | 8.268 | 80.204 | 81.548 | 1.00 | 55.49 | C |
| ATOM | 20545 | OG | SER | K | 43 | 7.572 | 80.205 | 80.303 | 1.00 | 66.50 | O |
| ATOM | 20547 | C | SER | K | 43 | 7.117 | 82.389 | 81.945 | 1.00 | 45.27 | C |
| ATOM | 20548 | O | SER | K | 43 | 7.990 | 83.205 | 81.647 | 1.00 | 45.75 | O |
| ATOM | 20549 | N | HIS | K | 44 | 5.831 | 82.643 | 81.751 | 1.00 | 45.61 | N |
| ATOM | 20551 | CA | HIS | K | 44 | 5.335 | 83.903 | 81.154 | 1.00 | 47.94 | C |
| ATOM | 20553 | CB | HIS | K | 44 | 5.728 | 85.139 | 81.958 | 1.00 | 48.85 | C |
| ATOM | 20556 | CG | HIS | K | 44 | 5.133 | 85.210 | 83.331 | 1.00 | 59.94 | C |
| ATOM | 20557 | ND1 | HIS | K | 44 | 4.265 | 84.261 | 83.826 | 1.00 | 63.62 | N |
| ATOM | 20559 | CE1 | HIS | K | 44 | 3.939 | 84.569 | 85.068 | 1.00 | 57.95 | C |
| ATOM | 20561 | NE2 | HIS | K | 44 | 4.539 | 85.702 | 85.382 | 1.00 | 63.90 | N |
| ATOM | 20563 | CD2 | HIS | K | 44 | 5.302 | 86.118 | 84.320 | 1.00 | 48.74 | C |
| ATOM | 20565 | C | HIS | K | 44 | 5.760 | 84.159 | 79.705 | 1.00 | 48.36 | C |
| ATOM | 20566 | O | HIS | K | 44 | 5.553 | 85.257 | 79.177 | 1.00 | 54.06 | O |
| ATOM | 20567 | N | VAL | K | 45 | 6.314 | 83.130 | 79.069 | 1.00 | 46.90 | N |
| ATOM | 20569 | CA | VAL | K | 45 | 6.624 | 83.099 | 77.639 | 1.00 | 42.57 | C |
| ATOM | 20571 | CB | VAL | K | 45 | 7.551 | 81.873 | 77.360 | 1.00 | 35.16 | C |
| ATOM | 20573 | CG1 | VAL | K | 45 | 7.464 | 81.458 | 75.949 | 1.00 | 49.68 | C |
| ATOM | 20577 | CG2 | VAL | K | 45 | 8.965 | 82.153 | 77.655 | 1.00 | 36.75 | C |
| ATOM | 20581 | C | VAL | K | 45 | 5.347 | 82.985 | 76.745 | 1.00 | 45.73 | C |
| ATOM | 20582 | O | VAL | K | 45 | 5.309 | 83.448 | 75.590 | 1.00 | 34.10 | O |
| ATOM | 20583 | N | SER | K | 46 | 4.307 | 82.309 | 77.223 | 1.00 | 45.61 | N |
| ATOM | 20585 | CA | SER | K | 46 | 3.194 | 82.008 | 76.336 | 1.00 | 46.07 | C |
| ATOM | 20587 | CB | SER | K | 46 | 3.136 | 80.517 | 75.980 | 1.00 | 52.67 | C |
| ATOM | 20590 | OG | SER | K | 46 | 2.611 | 79.745 | 77.035 | 1.00 | 45.18 | O |
| ATOM | 20592 | C | SER | K | 46 | 1.938 | 82.352 | 77.033 | 1.00 | 41.53 | C |
| ATOM | 20593 | O | SER | K | 46 | 1.890 | 82.304 | 78.242 | 1.00 | 41.34 | O |
| ATOM | 20594 | N | LEU | K | 47 | 0.880 | 82.579 | 76.280 | 1.00 | 44.75 | N |
| ATOM | 20596 | CA | LEU | K | 47 | −0.384 | 82.862 | 76.938 | 1.00 | 47.15 | C |
| ATOM | 20598 | CB | LEU | K | 47 | −0.578 | 84.369 | 77.062 | 1.00 | 48.09 | C |
| ATOM | 20601 | CG | LEU | K | 47 | −1.912 | 84.895 | 77.593 | 1.00 | 40.08 | C |
| ATOM | 20603 | CD1 | LEU | K | 47 | −1.773 | 86.222 | 78.321 | 1.00 | 46.10 | C |
| ATOM | 20607 | CD2 | LEU | K | 47 | −2.728 | 85.149 | 76.432 | 1.00 | 27.79 | C |
| ATOM | 20611 | C | LEU | K | 47 | −1.557 | 82.225 | 76.217 | 1.00 | 49.55 | C |
| ATOM | 20612 | O | LEU | K | 47 | −1.728 | 82.309 | 74.999 | 1.00 | 51.80 | O |
| ATOM | 20613 | N | VAL | K | 48 | −2.410 | 81.600 | 77.004 | 1.00 | 50.88 | N |
| ATOM | 20615 | CA | VAL | K | 48 | −3.579 | 81.007 | 76.406 | 1.00 | 53.35 | C |
| ATOM | 20617 | CB | VAL | K | 48 | −3.736 | 79.564 | 76.829 | 1.00 | 45.56 | C |
| ATOM | 20619 | CG1 | VAL | K | 48 | −4.042 | 79.502 | 78.269 | 1.00 | 53.57 | C |
| ATOM | 20623 | CG2 | VAL | K | 48 | −4.836 | 78.924 | 76.026 | 1.00 | 54.99 | C |
| ATOM | 20627 | C | VAL | K | 48 | −4.879 | 81.768 | 76.636 | 1.00 | 53.12 | C |
| ATOM | 20628 | O | VAL | K | 48 | −5.138 | 82.340 | 77.690 | 1.00 | 50.40 | O |
| ATOM | 20629 | N | GLN | K | 49 | −5.698 | 81.771 | 75.597 | 1.00 | 55.39 | N |
| ATOM | 20631 | CA | GLN | K | 49 | −6.984 | 82.429 | 75.653 | 1.00 | 48.87 | C |
| ATOM | 20633 | CB | GLN | K | 49 | −6.871 | 83.776 | 74.951 | 1.00 | 57.29 | C |
| ATOM | 20636 | CG | GLN | K | 49 | −7.806 | 84.882 | 75.478 | 1.00 | 69.02 | C |
| ATOM | 20639 | CD | GLN | K | 49 | −9.062 | 84.947 | 74.642 | 1.00 | 80.56 | C |
| ATOM | 20640 | OE1 | GLN | K | 49 | −9.028 | 85.407 | 73.484 | 1.00 | 73.55 | O |
| ATOM | 20641 | NE2 | GLN | K | 49 | −10.142 | 84.371 | 75.172 | 1.00 | 63.44 | N |
| ATOM | 20644 | C | GLN | K | 49 | −8.084 | 81.567 | 75.043 | 1.00 | 43.00 | C |
| ATOM | 20645 | O | GLN | K | 49 | −8.092 | 81.309 | 73.837 | 1.00 | 37.89 | O |
| ATOM | 20646 | N | LEU | K | 50 | −8.971 | 81.104 | 75.929 | 1.00 | 44.79 | N |
| ATOM | 20648 | CA | LEU | K | 50 | −10.172 | 80.306 | 75.647 | 1.00 | 45.37 | C |
| ATOM | 20650 | CB | LEU | K | 50 | −10.448 | 79.339 | 76.810 | 1.00 | 48.77 | C |
| ATOM | 20653 | CG | LEU | K | 50 | −11.700 | 78.462 | 76.689 | 1.00 | 39.54 | C |
| ATOM | 20655 | CD1 | LEU | K | 50 | −11.637 | 77.549 | 75.469 | 1.00 | 44.85 | C |
| ATOM | 20659 | CD2 | LEU | K | 50 | −11.928 | 77.639 | 77.918 | 1.00 | 46.54 | C |
| ATOM | 20663 | C | LEU | K | 50 | −11.430 | 81.141 | 75.559 | 1.00 | 51.07 | C |
| ATOM | 20664 | O | LEU | K | 50 | −11.734 | 81.890 | 76.490 | 1.00 | 51.57 | O |
| ATOM | 20665 | N | THR | K | 51 | −12.223 | 80.891 | 74.520 | 1.00 | 56.36 | N |
| ATOM | 20667 | CA | THR | K | 51 | −13.556 | 81.481 | 74.407 | 1.00 | 60.27 | C |
| ATOM | 20669 | CB | THR | K | 51 | −13.554 | 82.579 | 73.332 | 1.00 | 62.17 | C |
| ATOM | 20671 | OG1 | THR | K | 51 | −12.835 | 83.731 | 73.807 | 1.00 | 62.45 | O |
| ATOM | 20673 | CG2 | THR | K | 51 | −14.972 | 83.054 | 73.121 | 1.00 | 53.53 | C |
| ATOM | 20677 | C | THR | K | 51 | −14.668 | 80.483 | 74.081 | 1.00 | 58.76 | C |
| ATOM | 20678 | O | THR | K | 51 | −14.732 | 79.954 | 72.972 | 1.00 | 61.31 | O |
| ATOM | 20679 | N | LEU | K | 52 | −15.595 | 80.314 | 75.019 | 1.00 | 54.14 | N |
| ATOM | 20681 | CA | LEU | K | 52 | −16.815 | 79.565 | 74.764 | 1.00 | 51.95 | C |
| ATOM | 20683 | CB | LEU | K | 52 | −16.908 | 78.402 | 75.742 | 1.00 | 49.81 | C |
| ATOM | 20686 | CG | LEU | K | 52 | −15.676 | 77.514 | 75.727 | 1.00 | 48.50 | C |
| ATOM | 20688 | CD1 | LEU | K | 52 | −15.794 | 76.550 | 76.881 | 1.00 | 50.17 | C |
| ATOM | 20692 | CD2 | LEU | K | 52 | −15.595 | 76.781 | 74.403 | 1.00 | 51.38 | C |
| ATOM | 20696 | C | LEU | K | 52 | −18.057 | 80.447 | 74.893 | 1.00 | 50.65 | C |
| ATOM | 20697 | O | LEU | K | 52 | −18.385 | 80.922 | 75.972 | 1.00 | 49.62 | O |
| ATOM | 20698 | N | ARG | K | 53 | −18.748 | 80.672 | 73.782 | 1.00 | 53.36 | N |
| ATOM | 20700 | CA | ARG | K | 53 | −19.782 | 81.692 | 73.739 | 1.00 | 51.74 | C |
| ATOM | 20702 | CB | ARG | K | 53 | −20.062 | 82.128 | 72.301 | 1.00 | 49.53 | C |
| ATOM | 20705 | CG | ARG | K | 53 | −19.319 | 83.387 | 71.889 | 1.00 | 58.97 | C |
| ATOM | 20708 | CD | ARG | K | 53 | −19.539 | 83.797 | 70.426 | 1.00 | 79.61 | C |
| ATOM | 20711 | NE | ARG | K | 53 | −18.279 | 83.752 | 69.680 | 1.00 | 104.27 | N |
| ATOM | 20713 | CZ | ARG | K | 53 | −17.952 | 82.818 | 68.788 | 1.00 | 106.50 | C |
| ATOM | 20714 | NH1 | ARG | K | 53 | −18.803 | 81.848 | 68.470 | 1.00 | 99.76 | N |
| ATOM | 20717 | NH2 | ARG | K | 53 | −16.762 | 82.856 | 68.202 | 1.00 | 106.18 | N |
| ATOM | 20720 | C | ARG | K | 53 | −20.992 | 80.990 | 74.309 | 1.00 | 56.92 | C |
| ATOM | 20721 | O | ARG | K | 53 | −21.174 | 79.812 | 74.027 | 1.00 | 52.19 | O |
| ATOM | 20722 | N | SER | K | 54 | −21.801 | 81.700 | 75.099 | 1.00 | 59.99 | N |
| ATOM | 20724 | CA | SER | K | 54 | −23.001 | 81.124 | 75.720 | 1.00 | 58.25 | C |
| ATOM | 20726 | CB | SER | K | 54 | −23.768 | 82.183 | 76.504 | 1.00 | 53.65 | C |
| ATOM | 20729 | OG | SER | K | 54 | −24.193 | 83.180 | 75.599 | 1.00 | 62.46 | O |
| ATOM | 20731 | C | SER | K | 54 | −23.975 | 80.493 | 74.732 | 1.00 | 53.59 | C |
| ATOM | 20732 | O | SER | K | 54 | −24.615 | 79.496 | 75.059 | 1.00 | 45.60 | O |
| ATOM | 20733 | N | GLU | K | 55 | −24.147 | 81.125 | 73.573 | 1.00 | 54.93 | N |
| ATOM | 20735 | CA | GLU | K | 55 | −25.013 | 80.594 | 72.513 | 1.00 | 54.91 | C |
| ATOM | 20737 | CB | GLU | K | 55 | −24.886 | 81.446 | 71.252 | 1.00 | 55.82 | C |
| ATOM | 20740 | CG | GLU | K | 55 | −25.240 | 82.901 | 71.480 | 1.00 | 67.89 | C |
| ATOM | 20743 | CD | GLU | K | 55 | −23.994 | 83.742 | 71.500 | 1.00 | 76.83 | C |
| ATOM | 20744 | OE1 | GLU | K | 55 | −23.479 | 84.018 | 70.384 | 1.00 | 71.24 | O |
| ATOM | 20745 | OE2 | GLU | K | 55 | −23.526 | 84.034 | 72.632 | 1.00 | 62.27 | O |
| ATOM | 20746 | C | GLU | K | 55 | −24.659 | 79.167 | 72.128 | 1.00 | 52.69 | C |
| ATOM | 20747 | O | GLU | K | 55 | −25.358 | 78.546 | 71.351 | 1.00 | 57.23 | O |
| ATOM | 20748 | N | GLY | K | 56 | −23.511 | 78.680 | 72.569 | 1.00 | 49.49 | N |
| ATOM | 20750 | CA | GLY | K | 56 | −22.900 | 77.552 | 71.909 | 1.00 | 45.18 | C |
| ATOM | 20753 | C | GLY | K | 56 | −23.306 | 76.377 | 72.730 | 1.00 | 43.19 | C |
| ATOM | 20754 | O | GLY | K | 56 | −23.328 | 75.234 | 72.263 | 1.00 | 50.92 | O |
| ATOM | 20755 | N | PHE | K | 57 | −23.643 | 76.703 | 73.969 | 1.00 | 50.50 | N |
| ATOM | 20757 | CA | PHE | K | 57 | −24.124 | 75.730 | 74.948 | 1.00 | 53.10 | C |
| ATOM | 20759 | CB | PHE | K | 57 | −24.011 | 76.274 | 76.376 | 1.00 | 52.60 | C |
| ATOM | 20762 | CG | PHE | K | 57 | −22.586 | 76.433 | 76.843 | 1.00 | 66.76 | C |
| ATOM | 20763 | CD1 | PHE | K | 57 | −21.805 | 77.507 | 76.423 | 1.00 | 52.35 | C |
| ATOM | 20765 | CE1 | PHE | K | 57 | −20.493 | 77.614 | 76.823 | 1.00 | 49.60 | C |
| ATOM | 20767 | CZ | PHE | K | 57 | −19.949 | 76.635 | 77.611 | 1.00 | 57.88 | C |
| ATOM | 20769 | CE2 | PHE | K | 57 | −20.708 | 75.546 | 78.002 | 1.00 | 45.01 | C |
| ATOM | 20771 | CD2 | PHE | K | 57 | −22.003 | 75.445 | 77.623 | 1.00 | 57.66 | C |
| ATOM | 20773 | C | PHE | K | 57 | −25.565 | 75.445 | 74.639 | 1.00 | 56.89 | C |
| ATOM | 20774 | O | PHE | K | 57 | −26.331 | 76.371 | 74.338 | 1.00 | 49.25 | O |
| ATOM | 20775 | N | ASP | K | 58 | −25.900 | 74.159 | 74.721 | 1.00 | 63.62 | N |
| ATOM | 20777 | CA | ASP | K | 58 | −27.286 | 73.708 | 74.802 | 1.00 | 63.14 | C |
| ATOM | 20779 | CB | ASP | K | 58 | −27.355 | 72.195 | 74.950 | 1.00 | 66.81 | C |
| ATOM | 20782 | CG | ASP | K | 58 | −27.321 | 71.512 | 73.632 | 1.00 | 62.96 | C |
| ATOM | 20783 | OD1 | ASP | K | 58 | −26.220 | 71.102 | 73.235 | 1.00 | 57.81 | O |
| ATOM | 20784 | OD2 | ASP | K | 58 | −28.336 | 71.404 | 72.918 | 1.00 | 83.92 | O |
| ATOM | 20785 | C | ASP | K | 58 | −28.121 | 74.343 | 75.896 | 1.00 | 56.83 | C |
| ATOM | 20786 | O | ASP | K | 58 | −29.310 | 74.577 | 75.699 | 1.00 | 60.01 | O |
| ATOM | 20787 | N | THR | K | 59 | −27.510 | 74.625 | 77.035 | 1.00 | 54.68 | N |
| ATOM | 20789 | CA | THR | K | 59 | −28.232 | 75.308 | 78.102 | 1.00 | 63.31 | C |
| ATOM | 20791 | CB | THR | K | 59 | −28.726 | 74.245 | 79.097 | 1.00 | 68.41 | C |
| ATOM | 20793 | OG1 | THR | K | 59 | −29.858 | 73.553 | 78.551 | 1.00 | 71.93 | O |
| ATOM | 20795 | CG2 | THR | K | 59 | −29.183 | 74.866 | 80.407 | 1.00 | 70.72 | C |
| ATOM | 20799 | C | THR | K | 59 | −27.271 | 76.259 | 78.798 | 1.00 | 63.96 | C |
| ATOM | 20800 | O | THR | K | 59 | −26.148 | 75.864 | 79.127 | 1.00 | 67.71 | O |
| ATOM | 20801 | N | TYR | K | 60 | −27.682 | 77.503 | 79.022 | 1.00 | 57.76 | N |
| ATOM | 20803 | CA | TYR | K | 60 | −26.775 | 78.443 | 79.659 | 1.00 | 52.29 | C |
| ATOM | 20805 | CB | TYR | K | 60 | −26.083 | 79.320 | 78.637 | 1.00 | 53.98 | C |
| ATOM | 20808 | CG | TYR | K | 60 | −24.846 | 79.993 | 79.205 | 1.00 | 60.95 | C |
| ATOM | 20809 | CD1 | TYR | K | 60 | −23.619 | 79.364 | 79.188 | 1.00 | 63.82 | C |
| ATOM | 20811 | CE1 | TYR | K | 60 | −22.512 | 79.953 | 79.735 | 1.00 | 53.34 | C |
| ATOM | 20813 | CZ | TYR | K | 60 | −22.595 | 81.195 | 80.288 | 1.00 | 49.56 | C |
| ATOM | 20814 | OH | TYR | K | 60 | −21.471 | 81.800 | 80.821 | 1.00 | 39.97 | O |
| ATOM | 20816 | CE2 | TYR | K | 60 | −23.800 | 81.831 | 80.325 | 1.00 | 52.20 | C |
| ATOM | 20818 | CD2 | TYR | K | 60 | −24.918 | 81.221 | 79.815 | 1.00 | 51.75 | C |
| ATOM | 20820 | C | TYR | K | 60 | −27.477 | 79.367 | 80.598 | 1.00 | 52.81 | C |
| ATOM | 20821 | O | TYR | K | 60 | −28.506 | 79.911 | 80.261 | 1.00 | 68.94 | O |
| ATOM | 20822 | N | ARG | K | 61 | −26.872 | 79.620 | 81.744 | 1.00 | 55.69 | N |
| ATOM | 20824 | CA | ARG | K | 81 | −27.521 | 80.381 | 82.794 | 1.00 | 54.07 | C |
| ATOM | 20826 | CB | ARG | K | 61 | −28.350 | 79.455 | 83.677 | 1.00 | 55.25 | C |
| ATOM | 20829 | CG | ARG | K | 61 | −29.101 | 80.178 | 84.806 | 1.00 | 69.62 | C |
| ATOM | 20832 | CD | ARG | K | 61 | −29.681 | 79.250 | 85.881 | 1.00 | 73.08 | C |
| ATOM | 20835 | NE | ARG | K | 61 | −30.086 | 79.951 | 87.096 | 1.00 | 66.05 | N |
| ATOM | 20837 | CZ | ARG | K | 61 | −30.553 | 79.351 | 88.184 | 1.00 | 63.09 | C |
| ATOM | 20838 | NH1 | ARG | K | 61 | −30.633 | 78.032 | 88.258 | 1.00 | 61.14 | N |
| ATOM | 20841 | NH2 | ARG | K | 61 | −30.893 | 80.071 | 89.239 | 1.00 | 62.88 | N |
| ATOM | 20844 | C | ARG | K | 61 | −26.439 | 81.003 | 83.650 | 1.00 | 52.41 | C |
| ATOM | 20845 | O | ARG | K | 61 | −25.537 | 80.314 | 84.131 | 1.00 | 50.53 | O |
| ATOM | 20846 | N | CYS | K | 62 | −26.548 | 82.301 | 83.882 | 1.00 | 51.30 | N |
| ATOM | 20848 | CA | CYS | K | 62 | −25.547 | 82.962 | 84.687 | 1.00 | 52.73 | C |
| ATOM | 20850 | CB | CYS | K | 62 | −24.430 | 83.486 | 83.783 | 1.00 | 58.15 | C |
| ATOM | 20853 | SG | CYS | K | 62 | −23.055 | 84.228 | 84.709 | 1.00 | 64.52 | S |
| ATOM | 20854 | C | CYS | K | 62 | −26.144 | 84.139 | 85.390 | 1.00 | 44.11 | C |
| ATOM | 20855 | O | CYS | K | 62 | −26.157 | 85.198 | 84.812 | 1.00 | 51.00 | O |
| ATOM | 20856 | N | ASP | K | 63 | −26.593 | 83.977 | 86.627 | 1.00 | 53.32 | N |
| ATOM | 20858 | CA | ASP | K | 63 | −27.251 | 85.059 | 87.377 | 1.00 | 58.01 | C |
| ATOM | 20860 | CB | ASP | K | 63 | −27.830 | 84.568 | 88.705 | 1.00 | 52.86 | C |
| ATOM | 20863 | CG | ASP | K | 63 | −28.759 | 83.409 | 88.517 | 1.00 | 58.71 | C |
| ATOM | 20864 | OD1 | ASP | K | 63 | −29.440 | 83.341 | 87.462 | 1.00 | 46.32 | O |
| ATOM | 20865 | OD2 | ASP | K | 63 | −28.848 | 82.518 | 89.378 | 1.00 | 66.90 | O |
| ATOM | 20866 | C | ASP | K | 63 | −26.272 | 86.166 | 87.690 | 1.00 | 61.16 | C |
| ATOM | 20867 | O | ASP | K | 63 | −26.640 | 87.336 | 87.679 | 1.00 | 70.50 | O |
| ATOM | 20868 | N | ARG | K | 64 | −25.025 | 85.797 | 87.953 | 1.00 | 61.59 | N |
| ATOM | 20870 | CA | ARG | K | 64 | −24.067 | 86.774 | 88.408 | 1.00 | 67.52 | C |
| ATOM | 20872 | CB | ARG | K | 64 | −24.167 | 86.869 | 89.929 | 1.00 | 70.92 | C |
| ATOM | 20875 | CG | ARG | K | 64 | −24.650 | 88.250 | 90.372 | 1.00 | 94.31 | C |
| ATOM | 20878 | CD | ARG | K | 64 | −24.087 | 88.711 | 91.706 | 1.00 | 108.84 | C |
| ATOM | 20881 | NE | ARG | K | 64 | −24.386 | 87.751 | 92.769 | 1.00 | 111.36 | N |
| ATOM | 20883 | CZ | ARG | K | 64 | −25.308 | 87.928 | 93.709 | 1.00 | 104.70 | C |
| ATOM | 20884 | NH1 | ARG | K | 64 | −26.051 | 89.028 | 93.740 | 1.00 | 107.15 | N |
| ATOM | 20887 | NH2 | ARG | K | 64 | −25.484 | 86.995 | 94.630 | 1.00 | 98.24 | N |
| ATOM | 20890 | C | ARG | K | 64 | −22.648 | 86.472 | 87.932 | 1.00 | 63.89 | C |
| ATOM | 20891 | O | ARG | K | 64 | −22.191 | 85.353 | 88.077 | 1.00 | 70.19 | O |
| ATOM | 20892 | N | ASN | K | 65 | −21.961 | 87.460 | 87.362 | 1.00 | 62.61 | N |
| ATOM | 20894 | CA | ASN | K | 65 | −20.557 | 87.325 | 86.994 | 1.00 | 62.06 | C |
| ATOM | 20896 | CB | ASN | K | 65 | −19.925 | 88.682 | 86.720 | 1.00 | 57.01 | C |
| ATOM | 20899 | CG | ASN | K | 65 | −20.273 | 89.206 | 85.341 | 1.00 | 66.00 | C |
| ATOM | 20900 | OD1 | ASN | K | 65 | −19.964 | 90.345 | 85.020 | 1.00 | 70.88 | O |
| ATOM | 20901 | ND2 | ASN | K | 65 | −20.942 | 88.390 | 84.530 | 1.00 | 48.81 | N |
| ATOM | 20904 | C | ASN | K | 65 | −19.688 | 86.618 | 88.013 | 1.00 | 64.90 | C |
| ATOM | 20905 | O | ASN | K | 65 | −19.641 | 87.021 | 89.162 | 1.00 | 71.09 | O |
| ATOM | 20906 | N | LEU | K | 66 | −18.930 | 85.626 | 87.553 | 1.00 | 65.13 | N |
| ATOM | 28908 | CA | LEU | K | 66 | −18.325 | 84.614 | 88.411 | 1.00 | 63.73 | C |
| ATOM | 20910 | CB | LEU | K | 66 | −19.020 | 83.287 | 88.118 | 1.00 | 62.36 | C |
| ATOM | 20913 | CG | LEU | K | 66 | −19.659 | 82.567 | 89.281 | 1.00 | 66.34 | C |
| ATOM | 20915 | CD1 | LEU | K | 66 | −19.975 | 81.186 | 88.768 | 1.00 | 77.83 | C |
| ATOM | 20919 | CD2 | LEU | K | 66 | −18.687 | 82.509 | 90.457 | 1.00 | 88.90 | C |
| ATOM | 20923 | C | LEU | K | 66 | −16.835 | 84.525 | 88.046 | 1.00 | 56.96 | C |
| ATOM | 20924 | O | LEU | K | 66 | −16.466 | 84.923 | 86.949 | 1.00 | 58.79 | O |
| ATOM | 20925 | N | ALA | K | 67 | −15.971 | 84.053 | 88.942 | 1.00 | 44.76 | N |
| ATOM | 20927 | CA | ALA | K | 67 | −14.555 | 83.927 | 88.596 | 1.00 | 46.70 | C |
| ATOM | 20929 | CB | ALA | K | 67 | −13.754 | 85.202 | 88.872 | 1.00 | 39.38 | C |
| ATOM | 20933 | C | ALA | K | 67 | −13.939 | 82.789 | 89.368 | 1.00 | 46.31 | C |
| ATOM | 20934 | O | ALA | K | 67 | −13.335 | 83.006 | 90.405 | 1.00 | 49.60 | O |
| ATOM | 20935 | N | MET | K | 68 | −14.021 | 81.594 | 88.803 | 1.00 | 43.39 | N |
| ATOM | 20937 | CA | MET | K | 68 | −13.654 | 80.419 | 89.540 | 1.00 | 45.44 | C |
| ATOM | 20939 | CB | MET | K | 68 | −14.514 | 79.232 | 89.087 | 1.00 | 53.67 | C |
| ATOM | 20942 | CG | MET | K | 68 | −16.021 | 79.477 | 88.991 | 1.00 | 54.32 | C |
| ATOM | 20945 | SD | MET | K | 68 | −16.881 | 77.942 | 88.535 | 1.00 | 57.07 | S |
| ATOM | 20946 | CE | MET | K | 68 | −16.511 | 77.952 | 86.777 | 1.00 | 56.21 | C |
| ATOM | 20950 | C | MET | K | 68 | −12.176 | 80.176 | 89.276 | 1.00 | 48.78 | C |
| ATOM | 20951 | O | MET | K | 68 | −11.675 | 80.253 | 88.139 | 1.00 | 52.83 | O |
| ATOM | 20952 | N | GLY | K | 69 | −11.456 | 79.869 | 90.335 | 1.00 | 45.10 | N |
| ATOM | 20954 | CA | GLY | K | 69 | −10.103 | 79.416 | 90.129 | 1.00 | 46.04 | C |
| ATOM | 20957 | C | GLY | K | 69 | −10.149 | 77.917 | 90.139 | 1.00 | 50.26 | C |
| ATOM | 20958 | O | GLY | K | 69 | −10.487 | 77.340 | 91.162 | 1.00 | 56.83 | O |
| ATOM | 20959 | N | VAL | K | 70 | −9.865 | 77.292 | 89.002 | 1.00 | 53.98 | N |
| ATOM | 20961 | CA | VAL | K | 70 | −9.843 | 75.842 | 88.912 | 1.00 | 48.02 | C |
| ATOM | 20963 | CB | VAL | K | 70 | −10.415 | 75.370 | 87.583 | 1.00 | 51.08 | C |
| ATOM | 20965 | CG1 | VAL | K | 70 | −10.249 | 73.889 | 87.452 | 1.00 | 64.73 | C |
| ATOM | 20969 | CG2 | VAL | K | 70 | −11.865 | 75.727 | 87.440 | 1.00 | 58.80 | C |
| ATOM | 20973 | C | VAL | K | 70 | −8.404 | 75.388 | 88.915 | 1.00 | 45.56 | C |
| ATOM | 20974 | O | VAL | K | 70 | −7.584 | 75.937 | 88.206 | 1.00 | 48.38 | O |
| ATOM | 20975 | N | ASN | K | 71 | −8.114 | 74.326 | 89.648 | 1.00 | 51.53 | N |
| ATOM | 20977 | CA | ASN | K | 71 | −6.966 | 73.485 | 89.341 | 1.00 | 50.07 | C |
| ATOM | 20979 | CB | ASN | K | 71 | −6.676 | 72.557 | 90.507 | 1.00 | 41.38 | C |
| ATOM | 20982 | CG | ASN | K | 71 | −5.386 | 71.778 | 90.321 | 1.00 | 53.90 | C |
| ATOM | 20983 | OD1 | ASN | K | 71 | −4.985 | 71.492 | 89.192 | 1.00 | 53.50 | O |
| ATOM | 20984 | ND2 | ASN | K | 71 | −4.757 | 71.383 | 91.435 | 1.00 | 39.83 | N |
| ATOM | 20987 | C | ASN | K | 71 | −7.228 | 72.636 | 88.115 | 1.00 | 46.67 | C |
| ATOM | 20988 | O | ASN | K | 71 | −8.036 | 71.734 | 88.192 | 1.00 | 56.23 | O |
| ATOM | 20989 | N | LEU | K | 72 | −6.500 | 72.878 | 87.031 | 1.00 | 45.22 | N |
| ATOM | 20991 | CA | LEU | K | 72 | −6.843 | 72.332 | 85.728 | 1.00 | 45.29 | C |
| ATOM | 20993 | CB | LEU | K | 72 | −6.200 | 73.106 | 84.577 | 1.00 | 45.71 | C |
| ATOM | 20996 | CG | LEU | K | 72 | −6.619 | 74.568 | 84.457 | 1.00 | 45.16 | C |
| ATOM | 20998 | CD1 | LEU | K | 72 | −5.782 | 75.222 | 83.390 | 1.00 | 51.21 | C |
| ATOM | 21002 | CD2 | LEU | K | 72 | −8.093 | 74.739 | 84.177 | 1.00 | 41.71 | C |
| ATOM | 21006 | C | LEU | K | 72 | −6.389 | 70.904 | 85.608 | 1.00 | 42.34 | C |
| ATOM | 21007 | O | LEU | K | 72 | −6.857 | 70.193 | 84.744 | 1.00 | 43.99 | O |
| ATOM | 21008 | N | THR | K | 73 | −5.465 | 70.483 | 86.455 | 1.00 | 50.47 | N |
| ATOM | 21010 | CA | THR | K | 73 | −5.068 | 69.079 | 86.460 | 1.00 | 53.89 | C |
| ATOM | 21012 | CB | THR | K | 73 | −3.771 | 68.875 | 87.277 | 1.00 | 54.95 | C |
| ATOM | 21014 | OG1 | THR | K | 73 | −2.818 | 69.891 | 86.943 | 1.00 | 61.07 | O |
| ATOM | 21016 | CG2 | THR | K | 73 | −3.053 | 67.628 | 86.866 | 1.00 | 54.58 | C |
| ATOM | 21020 | C | THR | K | 73 | −6.243 | 68.341 | 87.079 | 1.00 | 54.59 | C |
| ATOM | 21021 | O | THR | K | 73 | −6.744 | 67.392 | 86.497 | 1.00 | 63.25 | O |
| ATOM | 21022 | N | SER | K | 74 | −6.750 | 68.838 | 88.203 | 1.00 | 57.64 | N |
| ATOM | 21024 | CA | SER | K | 74 | −7.870 | 68.193 | 88.891 | 1.00 | 56.85 | C |
| ATOM | 21026 | CB | SER | K | 74 | −8.201 | 68.931 | 90.198 | 1.00 | 56.12 | C |
| ATOM | 21029 | OG | SER | K | 74 | −7.031 | 69.078 | 90.992 | 1.00 | 58.04 | O |
| ATOM | 21031 | C | SER | K | 74 | −9.116 | 68.081 | 87.997 | 1.00 | 53.51 | C |
| ATOM | 21032 | O | SER | K | 74 | −9.730 | 67.007 | 87.864 | 1.00 | 44.51 | O |
| ATOM | 21033 | N | MET | K | 75 | −9.476 | 69.197 | 87.372 | 1.00 | 43.73 | N |
| ATOM | 21035 | CA | MET | K | 75 | −10.566 | 69.198 | 86.399 | 1.00 | 45.50 | C |
| ATOM | 21037 | CB | MET | K | 75 | −10.741 | 70.623 | 85.863 | 1.00 | 38.68 | C |
| ATOM | 21040 | CG | MET | K | 75 | −11.898 | 70.829 | 84.927 | 1.00 | 43.44 | C |
| ATOM | 21043 | SD | MET | K | 75 | −12.446 | 72.531 | 84.792 | 1.00 | 47.11 | S |
| ATOM | 21044 | CE | MET | K | 75 | −14.092 | 72.214 | 84.058 | 1.00 | 32.43 | C |
| ATOM | 21048 | C | MET | K | 75 | −10.409 | 68.166 | 85.256 | 1.00 | 42.30 | C |
| ATOM | 21049 | O | MET | K | 75 | −11.323 | 67.430 | 84.939 | 1.00 | 51.22 | O |
| ATOM | 21050 | N | SER | K | 76 | −9.246 | 68.120 | 84.630 | 1.00 | 45.26 | N |
| ATOM | 21052 | CA | SER | K | 76 | −8.959 | 67.179 | 83.564 | 1.00 | 43.58 | C |
| ATOM | 21054 | CB | SER | K | 76 | −7.468 | 67.255 | 83.195 | 1.00 | 43.23 | C |
| ATOM | 21057 | OG | SER | K | 76 | −7.133 | 66.412 | 82.100 | 1.00 | 56.69 | O |
| ATOM | 21059 | C | SER | K | 76 | −9.265 | 65.791 | 84.093 | 1.00 | 51.46 | C |
| ATOM | 21060 | O | SER | K | 76 | −9.958 | 65.016 | 83.421 | 1.00 | 50.00 | O |
| ATOM | 21061 | N | LYS | K | 77 | −8.707 | 65.451 | 85.259 | 1.00 | 46.08 | N |
| ATOM | 21063 | CA | LYS | K | 77 | −8.835 | 64.081 | 85.735 | 1.00 | 48.18 | C |
| ATOM | 21065 | CB | LYS | K | 77 | −8.140 | 63.861 | 87.077 | 1.00 | 52.33 | C |
| ATOM | 21068 | CG | LYS | K | 77 | −6.633 | 63.881 | 87.093 | 1.00 | 54.41 | C |
| ATOM | 21071 | CD | LYS | K | 77 | −6.229 | 63.885 | 88.567 | 1.00 | 74.77 | C |
| ATOM | 21074 | CE | LYS | K | 77 | −4.802 | 63.380 | 88.823 | 1.00 | 87.87 | C |
| ATOM | 21077 | NZ | LYS | K | 77 | −4.522 | 63.149 | 90.280 | 1.00 | 77.97 | N |
| ATOM | 21081 | C | LYS | K | 77 | −10.326 | 63.727 | 85.867 | 1.00 | 47.81 | C |
| ATOM | 21082 | O | LYS | K | 77 | −10.739 | 62.658 | 85.413 | 1.00 | 53.42 | O |
| ATOM | 21083 | N | ILE | K | 78 | −11.122 | 64.619 | 86.464 | 1.00 | 44.53 | N |
| ATOM | 21085 | CA | ILE | K | 78 | −12.567 | 64.472 | 86.554 | 1.00 | 39.01 | C |
| ATOM | 21087 | CB | ILE | K | 78 | −13.164 | 65.681 | 87.266 | 1.00 | 40.92 | C |
| ATOM | 21089 | CG1 | ILE | K | 78 | −13.293 | 65.424 | 88.758 | 1.00 | 46.72 | C |
| ATOM | 21092 | CD1 | ILE | K | 78 | −12.948 | 66.644 | 89.594 | 1.00 | 51.48 | C |
| ATOM | 21096 | CG2 | ILE | K | 78 | −14.547 | 65.954 | 86.782 | 1.00 | 44.83 | C |
| ATOM | 21100 | C | ILE | K | 78 | −13.223 | 64.349 | 85.177 | 1.00 | 46.43 | C |
| ATOM | 21101 | O | ILE | K | 78 | −14.096 | 63.479 | 84.946 | 1.00 | 49.95 | O |
| ATOM | 21102 | N | LEU | K | 79 | −12.791 | 65.191 | 84.241 | 1.00 | 42.52 | N |
| ATOM | 21104 | CA | LEU | K | 79 | −13.375 | 65.140 | 82.895 | 1.00 | 47.43 | C |
| ATOM | 21106 | CB | LEU | K | 79 | −13.000 | 66.336 | 82.024 | 1.00 | 45.19 | C |
| ATOM | 21109 | CG | LEU | K | 79 | −13.821 | 67.603 | 82.232 | 1.00 | 47.12 | C |
| ATOM | 21111 | CD1 | LEU | K | 79 | −13.387 | 68.597 | 81.190 | 1.00 | 61.89 | C |
| ATOM | 21115 | CD2 | LEU | K | 79 | −15.286 | 67.287 | 82.031 | 1.00 | 61.11 | C |
| ATOM | 21119 | C | LEU | K | 79 | −13.042 | 63.867 | 82.151 | 1.00 | 41.80 | C |
| ATOM | 21120 | O | LEU | K | 79 | −13.715 | 63.518 | 81.201 | 1.00 | 39.74 | O |
| ATOM | 21121 | N | LYS | K | 80 | −12.002 | 63.180 | 82.605 | 1.00 | 49.47 | N |
| ATOM | 21123 | CA | LYS | K | 80 | −11.566 | 61.964 | 81.964 | 1.00 | 51.95 | C |
| ATOM | 21125 | CB | LYS | K | 80 | −10.128 | 61.617 | 82.355 | 1.00 | 58.53 | C |
| ATOM | 21128 | CG | LYS | K | 80 | −9.057 | 62.453 | 81.645 | 1.00 | 67.50 | C |
| ATOM | 21131 | CD | LYS | K | 80 | −7.765 | 61.667 | 81.414 | 1.00 | 86.31 | C |
| ATOM | 21134 | CE | LYS | K | 80 | −7.177 | 61.932 | 80.007 | 1.00 | 94.53 | C |
| ATOM | 21137 | NZ | LYS | K | 80 | −6.850 | 60.699 | 79.191 | 1.00 | 87.72 | N |
| ATOM | 21141 | C | LYS | K | 80 | −12.523 | 60.883 | 82.400 | 1.00 | 51.97 | C |
| ATOM | 21142 | O | LYS | K | 80 | −12.394 | 59.755 | 81.964 | 1.00 | 57.65 | O |
| ATOM | 21143 | N | CYS | K | 81 | −13.497 | 61.226 | 83.232 | 1.00 | 55.37 | N |
| ATOM | 21145 | CA | CYS | K | 81 | −14.460 | 60.241 | 83.707 | 1.00 | 54.11 | C |
| ATOM | 21147 | CB | CYS | K | 81 | −14.818 | 60.480 | 85.167 | 1.00 | 53.34 | C |
| ATOM | 21150 | SG | CYS | K | 81 | −13.535 | 60.004 | 86.320 | 1.00 | 50.12 | S |
| ATOM | 21151 | C | CYS | K | 81 | −15.753 | 60.384 | 82.949 | 1.00 | 56.24 | C |
| ATOM | 21152 | O | CYS | K | 81 | −16.699 | 59.670 | 83.264 | 1.00 | 64.09 | O |
| ATOM | 21153 | N | ALA | K | 82 | −15.839 | 61.374 | 82.065 | 1.00 | 48.91 | N |
| ATOM | 21155 | CA | ALA | K | 82 | −16.937 | 61.436 | 81.104 | 1.00 | 49.02 | C |
| ATOM | 21157 | CB | ALA | K | 82 | −17.168 | 62.836 | 80.681 | 1.00 | 48.79 | C |
| ATOM | 21161 | C | ALA | K | 82 | −16.622 | 60.629 | 79.855 | 1.00 | 51.26 | C |
| ATOM | 21162 | O | ALA | K | 82 | −15.487 | 60.633 | 79.414 | 1.00 | 54.34 | O |
| ATOM | 21163 | N | GLY | K | 83 | −17.629 | 59.985 | 79.270 | 1.00 | 53.68 | N |
| ATOM | 21165 | CA | GLY | K | 83 | −17.561 | 59.508 | 77.897 | 1.00 | 58.73 | C |
| ATOM | 21168 | C | GLY | K | 83 | −17.557 | 60.615 | 76.851 | 1.00 | 63.67 | C |
| ATOM | 21169 | O | GLY | K | 83 | −17.763 | 61.799 | 77.204 | 1.00 | 65.01 | O |
| ATOM | 21170 | N | ASN | K | 84 | −17.300 | 60.249 | 75.589 | 1.00 | 58.08 | N |
| ATOM | 21172 | CA | ASN | K | 84 | −17.230 | 61.249 | 74.513 | 1.00 | 61.06 | C |
| ATOM | 21174 | CB | ASN | K | 84 | −16.406 | 60.774 | 73.299 | 1.00 | 61.96 | C |
| ATOM | 21177 | CG | ASN | K | 84 | −14.870 | 60.791 | 73.532 | 1.00 | 72.76 | C |
| ATOM | 21178 | OD1 | ASN | K | 84 | −14.228 | 61.847 | 73.586 | 1.00 | 77.95 | O |
| ATOM | 21179 | ND2 | ASN | K | 84 | −14.275 | 59.604 | 73.582 | 1.00 | 74.43 | N |
| ATOM | 21182 | C | ASN | K | 84 | −18.671 | 61.561 | 74.108 | 1.00 | 61.49 | C |
| ATOM | 21183 | O | ASN | K | 84 | −19.067 | 62.713 | 73.827 | 1.00 | 58.71 | O |
| ATOM | 21184 | N | GLU | K | 85 | −19.498 | 60.530 | 74.206 | 1.00 | 63.52 | N |
| ATOM | 21186 | CA | GLU | K | 85 | −20.877 | 60.735 | 73.835 | 1.00 | 69.66 | C |
| ATOM | 21188 | CB | GLU | K | 85 | −21.425 | 59.481 | 73.141 | 1.00 | 74.79 | C |
| ATOM | 21191 | CG | GLU | K | 85 | −21.772 | 59.708 | 71.655 | 1.00 | 86.94 | C |
| ATOM | 21194 | CD | GLU | K | 85 | −20.752 | 59.193 | 70.628 | 1.00 | 100.42 | C |
| ATOM | 21195 | OE1 | GLU | K | 85 | −19.572 | 59.624 | 70.637 | 1.00 | 96.82 | O |
| ATOM | 21196 | OE2 | GLU | K | 85 | −21.138 | 58.393 | 69.739 | 1.00 | 102.11 | O |
| ATOM | 21197 | C | GLU | K | 85 | −21.712 | 61.351 | 74.982 | 1.00 | 68.13 | C |
| ATOM | 21198 | O | GLU | K | 85 | −22.793 | 61.923 | 74.743 | 1.00 | 63.21 | O |
| ATOM | 21199 | N | ASP | K | 86 | −21.087 | 61.446 | 76.163 | 1.00 | 64.07 | N |
| ATOM | 21201 | CA | ASP | K | 86 | −21.701 | 62.011 | 77.372 | 1.00 | 54.45 | C |
| ATOM | 21203 | CB | ASP | K | 86 | −20.744 | 61.978 | 78.550 | 1.00 | 53.02 | C |
| ATOM | 21206 | CG | ASP | K | 86 | −20.929 | 60.760 | 79.414 | 1.00 | 51.39 | C |
| ATOM | 21207 | OD1 | ASP | K | 86 | −21.597 | 59.809 | 78.956 | 1.00 | 71.02 | O |
| ATOM | 21208 | OD2 | ASP | K | 86 | −20.407 | 60.657 | 80.547 | 1.00 | 66.65 | O |
| ATOM | 21209 | C | ASP | K | 86 | −22.160 | 63.445 | 77.237 | 1.00 | 56.75 | C |
| ATOM | 21210 | O | ASP | K | 86 | −21.556 | 64.233 | 76.498 | 1.00 | 61.63 | O |
| ATOM | 21211 | N | ILE | K | 87 | −23.219 | 63.756 | 77.984 | 1.00 | 49.32 | N |
| ATOM | 21213 | CA | ILE | K | 87 | −23.694 | 65.113 | 78.138 | 1.00 | 50.89 | C |
| ATOM | 21215 | CB | ILE | K | 87 | −25.204 | 65.160 | 78.423 | 1.00 | 56.09 | C |
| ATOM | 21217 | CG1 | ILE | K | 87 | −26.045 | 65.003 | 77.157 | 1.00 | 45.90 | C |
| ATOM | 21220 | CD1 | ILE | K | 87 | −26.991 | 63.805 | 77.214 | 1.00 | 55.84 | C |
| ATOM | 21224 | CG2 | ILE | K | 87 | −25.560 | 66.499 | 79.069 | 1.00 | 69.83 | C |
| ATOM | 21228 | C | ILE | K | 87 | −23.014 | 65.590 | 79.407 | 1.00 | 54.66 | C |
| ATOM | 21229 | O | ILE | K | 87 | −23.054 | 64.904 | 80.444 | 1.00 | 48.23 | O |
| ATOM | 21230 | N | ILE | K | 88 | −22.477 | 66.805 | 79.344 | 1.00 | 52.61 | N |
| ATOM | 21232 | CA | ILE | K | 88 | −21.800 | 67.358 | 80.492 | 1.00 | 49.56 | C |
| ATOM | 21234 | CB | ILE | K | 88 | −20.302 | 67.522 | 80.223 | 1.00 | 54.93 | C |
| ATOM | 21236 | CG1 | ILE | K | 88 | −19.673 | 66.169 | 79.868 | 1.00 | 38.09 | C |
| ATOM | 21239 | CD1 | ILE | K | 88 | −18.309 | 66.324 | 79.259 | 1.00 | 42.87 | C |
| ATOM | 21243 | CG2 | ILE | K | 88 | −19.641 | 68.187 | 81.426 | 1.00 | 48.84 | C |
| ATOM | 21247 | C | ILE | K | 88 | −22.397 | 68.690 | 80.902 | 1.00 | 53.54 | C |
| ATOM | 21248 | O | ILE | K | 88 | −22.572 | 69.608 | 80.100 | 1.00 | 45.55 | O |
| ATOM | 21249 | N | THR | K | 89 | −22.638 | 68.770 | 82.204 | 1.00 | 60.49 | N |
| ATOM | 21251 | CA | THR | K | 89 | −23.265 | 69.905 | 82.833 | 1.00 | 65.05 | C |
| ATOM | 21253 | CB | THR | K | 89 | −24.600 | 69.444 | 83.423 | 1.00 | 67.70 | C |
| ATOM | 21255 | OG1 | THR | K | 89 | −25.225 | 68.551 | 82.492 | 1.00 | 77.26 | O |
| ATOM | 21257 | CG2 | THR | K | 89 | −25.593 | 70.594 | 83.487 | 1.00 | 76.10 | C |
| ATOM | 21261 | C | THR | K | 89 | −22.323 | 70.496 | 83.878 | 1.00 | 62.04 | C |
| ATOM | 21262 | O | THR | K | 89 | −21.706 | 69.781 | 84.672 | 1.00 | 59.84 | O |
| ATOM | 21263 | N | LEU | K | 90 | −22.157 | 71.810 | 83.781 | 1.00 | 59.20 | N |
| ATOM | 21265 | CA | LEU | K | 90 | −21.283 | 72.572 | 84.657 | 1.00 | 58.34 | C |
| ATOM | 21267 | CB | LEU | K | 90 | −20.325 | 73.450 | 83.836 | 1.00 | 51.34 | C |
| ATOM | 21270 | CG | LEU | K | 90 | −19.200 | 72.757 | 83.058 | 1.00 | 50.60 | C |
| ATOM | 21272 | CD1 | LEU | K | 90 | −18.193 | 73.791 | 82.615 | 1.00 | 44.64 | C |
| ATOM | 21276 | CD2 | LEU | K | 90 | −18.501 | 71.627 | 83.836 | 1.00 | 57.90 | C |
| ATOM | 21280 | C | LEU | K | 90 | −22.192 | 73.451 | 85.521 | 1.00 | 64.01 | C |
| ATOM | 21281 | O | LEU | K | 90 | −22.844 | 74.362 | 85.009 | 1.00 | 66.99 | O |
| ATOM | 21282 | N | ARG | K | 91 | −22.231 | 73.187 | 86.826 | 1.00 | 66.46 | N |
| ATOM | 21284 | CA | ARG | K | 91 | −23.032 | 73.993 | 87.740 | 1.00 | 65.24 | C |
| ATOM | 21286 | CB | ARG | K | 91 | −24.220 | 73.187 | 88.275 | 1.00 | 69.95 | C |
| ATOM | 21289 | CG | ARG | K | 91 | −25.349 | 73.983 | 88.959 | 1.00 | 87.41 | C |
| ATOM | 21292 | CD | ARG | K | 91 | −25.874 | 73.345 | 90.259 | 1.00 | 106.54 | C |
| ATOM | 21295 | NE | ARG | K | 91 | −25.772 | 71.888 | 90.209 | 1.00 | 120.86 | N |
| ATOM | 21297 | CZ | ARG | K | 91 | −26.656 | 71.108 | 89.601 | 1.00 | 135.21 | C |
| ATOM | 21298 | NH1 | ARG | K | 91 | −27.746 | 71.637 | 89.045 | 1.00 | 140.78 | N |
| ATOM | 21301 | NH2 | ARG | K | 91 | −26.456 | 69.796 | 89.561 | 1.00 | 132.47 | N |
| ATOM | 21304 | C | ARG | K | 91 | −22.149 | 74.508 | 88.865 | 1.00 | 58.27 | C |
| ATOM | 21305 | O | ARG | K | 91 | −21.205 | 73.834 | 89.292 | 1.00 | 46.93 | O |
| ATOM | 21306 | N | ALA | K | 92 | −22.401 | 75.772 | 89.204 | 1.00 | 57.45 | N |
| ATOM | 21308 | CA | ALA | K | 92 | −21.850 | 76.435 | 90.391 | 1.00 | 58.68 | C |
| ATOM | 21310 | CB | ALA | K | 92 | −20.424 | 76.907 | 90.140 | 1.00 | 54.06 | C |
| ATOM | 21314 | C | ALA | K | 92 | −22.710 | 77.603 | 90.913 | 1.00 | 58.84 | C |
| ATOM | 21315 | O | ALA | K | 92 | −23.332 | 78.373 | 90.163 | 1.00 | 56.73 | O |
| ATOM | 21316 | N | GLU | K | 93 | −22.710 | 77.727 | 92.234 | 1.00 | 59.52 | N |
| ATOM | 21318 | CA | GLU | K | 93 | −23.327 | 78.845 | 92.923 | 1.00 | 67.90 | C |
| ATOM | 21320 | CB | GLU | K | 93 | −23.385 | 78.492 | 94.404 | 1.00 | 70.04 | C |
| ATOM | 21323 | CG | GLU | K | 93 | −23.866 | 77.073 | 94.643 | 1.00 | 67.32 | C |
| ATOM | 21326 | CD | GLU | K | 93 | −25.261 | 77.054 | 95.206 | 1.00 | 78.68 | C |
| ATOM | 21327 | OE1 | GLU | K | 93 | −26.061 | 77.940 | 94.836 | 1.00 | 90.41 | O |
| ATOM | 21328 | OE2 | GLU | K | 93 | −25.526 | 76.185 | 96.057 | 1.00 | 92.76 | O |
| ATOM | 21329 | C | GLU | K | 93 | −22.554 | 80.151 | 92.753 | 1.00 | 76.55 | C |
| ATOM | 21330 | O | GLU | K | 93 | −21.340 | 80.144 | 92.604 | 1.00 | 84.38 | O |
| ATOM | 21331 | N | ASP | K | 94 | −23.243 | 81.280 | 92.861 | 1.00 | 85.70 | N |
| ATOM | 21333 | CA | ASP | K | 94 | −22.605 | 82.591 | 92.884 | 1.00 | 89.09 | C |
| ATOM | 21335 | CB | ASP | K | 94 | −23.690 | 83.647 | 93.078 | 1.00 | 85.40 | C |
| ATOM | 21338 | CG | ASP | K | 94 | −24.686 | 83.640 | 91.945 | 1.00 | 94.07 | C |
| ATOM | 21339 | OD1 | ASP | K | 94 | −25.610 | 84.478 | 91.956 | 1.00 | 108.86 | O |
| ATOM | 21340 | OD2 | ASP | K | 94 | −24.603 | 82.836 | 90.989 | 1.00 | 90.30 | O |
| ATOM | 21341 | C | ASP | K | 94 | −21.479 | 82.818 | 93.906 | 1.00 | 93.03 | C |
| ATOM | 21342 | O | ASP | K | 94 | −21.089 | 83.953 | 94.137 | 1.00 | 101.97 | O |
| ATOM | 21343 | N | ASN | K | 95 | −20.906 | 81.775 | 94.492 | 1.00 | 94.68 | N |
| ATOM | 21345 | CA | ASN | K | 95 | −19.807 | 81.977 | 95.432 | 1.00 | 94.33 | C |
| ATOM | 21347 | CB | ASN | K | 95 | −20.409 | 82.203 | 96.818 | 1.00 | 91.71 | C |
| ATOM | 21350 | CG | ASN | K | 95 | −21.708 | 81.437 | 97.005 | 1.00 | 101.46 | C |
| ATOM | 21351 | OD1 | ASN | K | 95 | −21.690 | 80.272 | 97.388 | 1.00 | 108.44 | O |
| ATOM | 21352 | ND2 | ASN | K | 95 | −22.833 | 82.057 | 96.665 | 1.00 | 110.60 | N |
| ATOM | 21355 | C | ASN | K | 95 | −18.836 | 80.786 | 95.445 | 1.00 | 96.56 | C |
| ATOM | 21356 | O | ASN | K | 95 | −18.987 | 79.869 | 96.261 | 1.00 | 97.39 | O |
| ATOM | 21357 | N | ALA | K | 96 | −17.861 | 80.786 | 94.531 | 1.00 | 94.47 | N |
| ATOM | 21359 | CA | ALA | K | 96 | −16.815 | 79.756 | 94.511 | 1.00 | 93.69 | C |
| ATOM | 21361 | CB | ALA | K | 96 | −15.395 | 80.367 | 94.803 | 1.00 | 94.07 | C |
| ATOM | 21365 | C | ALA | K | 96 | −17.133 | 78.621 | 95.499 | 1.00 | 88.46 | C |
| ATOM | 21366 | O | ALA | K | 96 | −18.279 | 78.169 | 95.598 | 1.00 | 81.70 | O |
| ATOM | 21367 | N | ASP | K | 97 | −16.103 | 78.169 | 96.216 | 1.00 | 84.73 | N |
| ATOM | 21369 | CA | ASP | K | 97 | −16.131 | 76.909 | 96.962 | 1.00 | 80.02 | C |
| ATOM | 21371 | CB | ASP | K | 97 | −17.469 | 76.707 | 97.679 | 1.00 | 81.93 | C |
| ATOM | 21374 | CG | ASP | K | 97 | −17.428 | 77.187 | 99.124 | 1.00 | 92.55 | C |
| ATOM | 21375 | OD1 | ASP | K | 97 | −16.548 | 78.030 | 99.397 | 1.00 | 73.03 | O |
| ATOM | 21376 | OD2 | ASP | K | 97 | −18.218 | 76.818 | 100.035 | 1.00 | 83.55 | O |
| ATOM | 21377 | C | ASP | K | 97 | −15.893 | 75.709 | 96.069 | 1.00 | 71.62 | C |
| ATOM | 21378 | O | ASP | K | 97 | −14.881 | 75.020 | 96.166 | 1.00 | 64.57 | O |
| ATOM | 21379 | N | THR | K | 98 | −16.877 | 75.451 | 95.221 | 1.00 | 64.50 | N |
| ATOM | 21381 | CA | THR | K | 98 | −17.090 | 74.116 | 94.712 | 1.00 | 59.53 | C |
| ATOM | 21383 | CB | THR | K | 98 | −17.980 | 73.338 | 95.680 | 1.00 | 56.81 | C |
| ATOM | 21385 | OG1 | THR | K | 98 | −17.303 | 72.134 | 96.058 | 1.00 | 60.84 | O |
| ATOM | 21387 | CG2 | THR | K | 98 | −19.261 | 72.888 | 94.985 | 1.00 | 63.67 | C |
| ATOM | 21391 | C | THR | K | 98 | −17.664 | 74.221 | 93.310 | 1.00 | 49.02 | C |
| ATOM | 21392 | O | THR | K | 98 | −18.253 | 75.237 | 92.956 | 1.00 | 43.43 | O |
| ATOM | 21393 | N | LEU | K | 99 | −17.328 | 73.250 | 92.463 | 1.00 | 50.50 | N |
| ATOM | 21395 | CA | LEU | K | 99 | −17.846 | 73.192 | 91.094 | 1.00 | 48.26 | C |
| ATOM | 21397 | CB | LEU | K | 99 | −16.786 | 73.499 | 90.045 | 1.00 | 48.88 | C |
| ATOM | 21400 | CG | LEU | K | 99 | −17.253 | 73.281 | 88.600 | 1.00 | 36.04 | C |
| ATOM | 21402 | CD1 | LEU | K | 99 | −18.106 | 74.415 | 87.995 | 1.00 | 29.80 | C |
| ATOM | 21406 | CD2 | LEU | K | 99 | −16.016 | 73.145 | 87.797 | 1.00 | 38.28 | C |
| ATOM | 21410 | C | LEU | K | 99 | −18.360 | 71.804 | 90.820 | 1.00 | 49.52 | C |
| ATOM | 21411 | O | LEU | K | 99 | −17.708 | 70.812 | 91.136 | 1.00 | 52.46 | O |
| ATOM | 21412 | N | ALA | K | 100 | −19.553 | 71.754 | 90.247 | 1.00 | 51.72 | N |
| ATOM | 21414 | CA | ALA | K | 100 | −20.231 | 70.496 | 90.011 | 1.00 | 51.45 | C |
| ATOM | 21416 | CB | ALA | K | 100 | −21.653 | 70.582 | 90.493 | 1.00 | 55.59 | C |
| ATOM | 21420 | C | ALA | K | 100 | −20.193 | 70.226 | 88.517 | 1.00 | 54.38 | C |
| ATOM | 21421 | O | ALA | K | 100 | −20.480 | 71.092 | 87.684 | 1.00 | 51.87 | O |
| ATOM | 21422 | N | LEU | K | 101 | −19.769 | 69.012 | 88.201 | 1.00 | 55.58 | N |
| ATOM | 21424 | CA | LEU | K | 101 | −19.787 | 68.498 | 86.841 | 1.00 | 53.90 | C |
| ATOM | 21426 | CB | LEU | K | 101 | −18.358 | 68.145 | 86.409 | 1.00 | 53.20 | C |
| ATOM | 21429 | CG | LEU | K | 101 | −17.311 | 69.213 | 86.756 | 1.00 | 51.79 | C |
| ATOM | 21431 | CD1 | LEU | K | 101 | −16.541 | 68.845 | 88.004 | 1.00 | 42.96 | C |
| ATOM | 21435 | CD2 | LEU | K | 101 | −16.325 | 69.411 | 85.626 | 1.00 | 69.52 | C |
| ATOM | 21439 | C | LEU | K | 101 | −20.672 | 67.256 | 86.850 | 1.00 | 49.87 | C |
| ATOM | 21440 | O | LEU | K | 101 | −20.492 | 66.350 | 87.690 | 1.00 | 46.53 | O |
| ATOM | 21441 | N | VAL | K | 102 | −21.650 | 67.249 | 85.943 | 1.00 | 49.00 | N |
| ATOM | 21443 | CA | VAL | K | 102 | −22.550 | 66.107 | 85.804 | 1.00 | 51.41 | C |
| ATOM | 21445 | CB | VAL | K | 102 | −24.035 | 66.445 | 86.036 | 1.00 | 51.89 | C |
| ATOM | 21447 | CG1 | VAL | K | 102 | −24.778 | 65.187 | 86.383 | 1.00 | 59.47 | C |
| ATOM | 21451 | CG2 | VAL | K | 102 | −24.221 | 67.448 | 87.149 | 1.00 | 65.50 | C |
| ATOM | 21455 | C | VAL | K | 102 | −22.434 | 65.482 | 84.429 | 1.00 | 49.77 | C |
| ATOM | 21456 | O | VAL | K | 102 | −22.654 | 66.147 | 83.424 | 1.00 | 52.82 | O |
| ATOM | 21457 | N | PHE | K | 103 | −22.164 | 64.179 | 84.410 | 1.00 | 53.36 | N |
| ATOM | 21459 | CA | PHE | K | 103 | −21.976 | 63.409 | 83.175 | 1.00 | 52.91 | C |
| ATOM | 21461 | CB | PHE | K | 103 | −20.679 | 62.615 | 83.286 | 1.00 | 48.26 | C |
| ATOM | 21464 | CG | PHE | K | 103 | −19.477 | 63.466 | 83.622 | 1.00 | 51.43 | C |
| ATOM | 21465 | CD1 | PHE | K | 103 | −19.415 | 64.790 | 83.240 | 1.00 | 33.19 | C |
| ATOM | 21467 | CE1 | PHE | K | 103 | −18.279 | 65.500 | 83.417 | 1.00 | 44.27 | C |
| ATOM | 21469 | CZ | PHE | K | 103 | −17.205 | 64.944 | 84.074 | 1.00 | 44.17 | C |
| ATOM | 21471 | CE2 | PHE | K | 103 | −17.247 | 63.649 | 84.461 | 1.00 | 46.90 | C |
| ATOM | 21473 | CD2 | PHE | K | 103 | −18.372 | 62.909 | 84.234 | 1.00 | 59.14 | C |
| ATOM | 21475 | C | PHE | K | 103 | −23.127 | 62.431 | 82.953 | 1.00 | 53.14 | C |
| ATOM | 21476 | O | PHE | K | 103 | −23.280 | 61.472 | 83.713 | 1.00 | 46.38 | O |
| ATOM | 21477 | N | GLU | K | 104 | −23.954 | 62.708 | 81.947 | 1.00 | 54.34 | N |
| ATOM | 21479 | CA | GLU | K | 104 | −25.125 | 61.880 | 81.650 | 1.00 | 58.12 | C |
| ATOM | 21481 | CB | GLU | K | 104 | −26.408 | 62.725 | 81.543 | 1.00 | 66.58 | C |
| ATOM | 21484 | CG | GLU | K | 104 | −26.741 | 63.632 | 82.727 | 1.00 | 77.82 | C |
| ATOM | 21487 | CD | GLU | K | 104 | −27.997 | 64.462 | 82.492 | 1.00 | 87.55 | C |
| ATOM | 21488 | OE1 | GLU | K | 104 | −29.094 | 63.864 | 82.594 | 1.00 | 80.10 | O |
| ATOM | 21489 | OE2 | GLU | K | 104 | −27.892 | 65.691 | 82.225 | 1.00 | 75.78 | O |
| ATOM | 21490 | C | GLU | K | 104 | −24.921 | 61.189 | 80.308 | 1.00 | 57.64 | C |
| ATOM | 21491 | O | GLU | K | 104 | −24.527 | 61.815 | 79.327 | 1.00 | 64.13 | O |
| ATOM | 21492 | N | ALA | K | 105 | −25.231 | 59.902 | 80.266 | 1.00 | 59.75 | N |
| ATOM | 21494 | CA | ALA | K | 105 | −24.917 | 59.052 | 79.134 | 1.00 | 60.54 | C |
| ATOM | 21496 | CB | ALA | K | 105 | −24.624 | 57.620 | 79.641 | 1.00 | 56.70 | C |
| ATOM | 21500 | C | ALA | K | 105 | −26.148 | 59.085 | 78.240 | 1.00 | 66.29 | C |
| ATOM | 21501 | O | ALA | K | 105 | −27.248 | 58.752 | 78.681 | 1.00 | 68.72 | O |
| ATOM | 21502 | N | PRO | K | 106 | −25.990 | 59.436 | 76.971 | 1.00 | 77.19 | N |
| ATOM | 21503 | CA | PRO | K | 106 | −27.155 | 59.876 | 76.197 | 1.00 | 80.78 | C |
| ATOM | 21505 | CB | PRO | K | 106 | −26.569 | 60.476 | 74.919 | 1.00 | 76.59 | C |
| ATOM | 21508 | CG | PRO | K | 106 | −25.138 | 60.026 | 74.888 | 1.00 | 81.74 | C |
| ATOM | 21511 | CD | PRO | K | 106 | −24.786 | 59.271 | 76.141 | 1.00 | 78.04 | C |
| ATOM | 21514 | C | PRO | K | 106 | −27.989 | 58.653 | 75.919 | 1.00 | 79.79 | C |
| ATOM | 21515 | O | PRO | K | 106 | −29.196 | 58.776 | 75.718 | 1.00 | 85.84 | O |
| ATOM | 21516 | N | ASN | K | 107 | −27.357 | 57.489 | 76.038 | 1.00 | 83.75 | N |
| ATOM | 21518 | CA | ASN | K | 107 | −27.993 | 56.214 | 75.712 | 1.00 | 89.86 | C |
| ATOM | 21520 | CB | ASN | K | 107 | −27.088 | 55.388 | 74.802 | 1.00 | 94.33 | C |
| ATOM | 21523 | CG | ASN | K | 107 | −27.311 | 55.692 | 73.336 | 1.00 | 99.08 | C |
| ATOM | 21524 | OD1 | ASN | K | 107 | −28.314 | 56.321 | 72.957 | 1.00 | 88.93 | O |
| ATOM | 21525 | ND2 | ASN | K | 107 | −26.364 | 55.260 | 72.501 | 1.00 | 84.08 | N |
| ATOM | 21528 | C | ASN | K | 107 | −28.354 | 55.356 | 76.909 | 1.00 | 86.78 | C |
| ATOM | 21529 | O | ASN | K | 107 | −29.380 | 54.701 | 76.915 | 1.00 | 93.63 | O |
| ATOM | 21530 | N | GLN | K | 108 | −27.499 | 55.315 | 77.914 | 1.00 | 86.55 | N |
| ATOM | 21532 | CA | GLN | K | 108 | −27.759 | 54.415 | 79.019 | 1.00 | 89.67 | C |
| ATOM | 21534 | CB | GLN | K | 108 | −26.523 | 53.566 | 79.347 | 1.00 | 89.78 | C |
| ATOM | 21537 | CG | GLN | K | 108 | −25.313 | 53.899 | 78.488 | 1.00 | 93.51 | C |
| ATOM | 21540 | CD | GLN | K | 108 | −24.035 | 53.671 | 79.245 | 1.00 | 92.82 | C |
| ATOM | 21541 | OE1 | GLN | K | 108 | −24.078 | 53.095 | 80.334 | 1.00 | 73.91 | O |
| ATOM | 21542 | NE2 | GLN | K | 108 | −22.899 | 54.098 | 78.678 | 1.00 | 91.36 | N |
| ATOM | 21545 | C | GLN | K | 108 | −28.337 | 55.130 | 80.256 | 1.00 | 88.96 | C |
| ATOM | 21546 | O | GLN | K | 108 | −28.441 | 56.373 | 80.315 | 1.00 | 88.68 | O |
| ATOM | 21547 | N | GLU | K | 109 | −28.781 | 54.286 | 81.191 | 1.00 | 81.13 | N |
| ATOM | 21549 | CA | GLU | K | 109 | −29.247 | 54.673 | 82.505 | 1.00 | 73.66 | C |
| ATOM | 21551 | CB | GLU | K | 109 | −30.186 | 53.577 | 83.017 | 1.00 | 79.69 | C |
| ATOM | 21554 | CG | GLU | K | 109 | −31.657 | 53.964 | 83.096 | 1.00 | 94.04 | C |
| ATOM | 21557 | CD | GLU | K | 109 | −32.076 | 54.789 | 81.909 | 1.00 | 102.91 | C |
| ATOM | 21558 | OE1 | GLU | K | 109 | −32.131 | 56.030 | 82.033 | 1.00 | 105.26 | O |
| ATOM | 21559 | OE2 | GLU | K | 109 | −32.290 | 54.174 | 80.848 | 1.00 | 120.23 | O |
| ATOM | 21560 | C | GLU | K | 109 | −28.032 | 54.787 | 83.422 | 1.00 | 67.45 | C |
| ATOM | 21561 | O | GLU | K | 109 | −27.841 | 53.986 | 84.346 | 1.00 | 69.11 | O |
| ATOM | 21562 | N | LYS | K | 110 | −27.197 | 55.783 | 83.142 | 1.00 | 56.52 | N |
| ATOM | 21564 | CA | LYS | K | 110 | −25.992 | 56.034 | 83.917 | 1.00 | 51.35 | C |
| ATOM | 21566 | CB | LYS | K | 110 | −24.798 | 55.456 | 83.187 | 1.00 | 46.46 | C |
| ATOM | 21569 | CG | LYS | K | 110 | −23.510 | 55.564 | 83.976 | 1.00 | 60.98 | C |
| ATOM | 21572 | CD | LYS | K | 110 | −22.243 | 55.409 | 83.096 | 1.00 | 59.09 | C |
| ATOM | 21575 | CE | LYS | K | 110 | −21.012 | 55.376 | 83.998 | 1.00 | 61.06 | C |
| ATOM | 21578 | NZ | LYS | K | 110 | −19.715 | 55.215 | 83.342 | 1.00 | 40.86 | N |
| ATOM | 21582 | C | LYS | K | 110 | −25.784 | 57.539 | 84.073 | 1.00 | 52.23 | C |
| ATOM | 21583 | O | LYS | K | 110 | −25.635 | 58.241 | 83.072 | 1.00 | 44.36 | O |
| ATOM | 21584 | N | VAL | K | 111 | −25.759 | 58.028 | 85.316 | 1.00 | 54.72 | N |
| ATOM | 21586 | CA | VAL | K | 111 | −25.342 | 59.406 | 85.597 | 1.00 | 56.97 | C |
| ATOM | 21588 | CB | VAL | K | 111 | −26.469 | 60.267 | 86.109 | 1.00 | 53.63 | C |
| ATOM | 21590 | CG1 | VAL | K | 111 | −25.893 | 61.558 | 86.614 | 1.00 | 60.57 | C |
| ATOM | 21594 | CG2 | VAL | K | 111 | −27.471 | 60.511 | 85.007 | 1.00 | 62.38 | C |
| ATOM | 21598 | C | VAL | K | 111 | −24.242 | 59.521 | 86.632 | 1.00 | 55.20 | C |
| ATOM | 21599 | O | VAL | K | 111 | −24.315 | 58.848 | 87.645 | 1.00 | 62.26 | O |
| ATOM | 21600 | N | SER | K | 112 | −23.227 | 60.340 | 86.350 | 1.00 | 58.04 | N |
| ATOM | 21602 | CA | SER | K | 112 | −22.097 | 60.582 | 87.250 | 1.00 | 60.09 | C |
| ATOM | 21604 | CB | SER | K | 112 | −20.780 | 60.358 | 86.509 | 1.00 | 63.75 | C |
| ATOM | 21607 | OG | SER | K | 112 | −20.575 | 58.994 | 86.212 | 1.00 | 75.29 | O |
| ATOM | 21609 | C | SER | K | 112 | −22.120 | 62.048 | 87.660 | 1.00 | 58.22 | C |
| ATOM | 21610 | O | SER | K | 112 | −22.361 | 62.908 | 86.804 | 1.00 | 56.89 | O |
| ATOM | 21611 | N | ASP | K | 113 | −21.870 | 62.314 | 88.945 | 1.00 | 52.19 | N |
| ATOM | 21613 | CA | ASP | K | 113 | −21.978 | 63.650 | 89.552 | 1.00 | 48.00 | C |
| ATOM | 21615 | CB | ASP | K | 113 | −23.085 | 63.704 | 90.622 | 1.00 | 47.42 | C |
| ATOM | 21618 | CG | ASP | K | 113 | −24.418 | 64.122 | 90.054 | 1.00 | 57.79 | C |
| ATOM | 21619 | OD1 | ASP | K | 113 | −24.626 | 65.341 | 89.848 | 1.00 | 81.76 | O |
| ATOM | 21620 | OD2 | ASP | K | 113 | −25.307 | 63.304 | 89.726 | 1.00 | 73.95 | O |
| ATOM | 21621 | C | ASP | K | 113 | −20.680 | 63.815 | 90.286 | 1.00 | 47.34 | C |
| ATOM | 21622 | O | ASP | K | 113 | −20.432 | 63.080 | 91.257 | 1.00 | 40.64 | O |
| ATOM | 21623 | N | TYR | K | 114 | −19.834 | 64.715 | 89.781 | 1.00 | 55.05 | N |
| ATOM | 21625 | CA | TYR | K | 114 | −18.477 | 64.914 | 90.331 | 1.00 | 53.19 | C |
| ATOM | 21627 | CB | TYR | K | 114 | −17.378 | 64.810 | 89.269 | 1.00 | 44.80 | C |
| ATOM | 21630 | CG | TYR | K | 114 | −17.160 | 63.392 | 88.852 | 1.00 | 48.96 | C |
| ATOM | 21631 | CD1 | TYR | K | 114 | −17.829 | 62.873 | 87.746 | 1.00 | 44.12 | C |
| ATOM | 21633 | CE1 | TYR | K | 114 | −17.681 | 61.562 | 87.371 | 1.00 | 33.90 | C |
| ATOM | 21635 | CZ | TYR | K | 114 | −16.864 | 60.742 | 88.099 | 1.00 | 43.70 | C |
| ATOM | 21636 | OH | TYR | K | 114 | −16.766 | 59.444 | 87.669 | 1.00 | 46.33 | O |
| ATOM | 21638 | CE2 | TYR | K | 114 | −16.213 | 61.207 | 89.232 | 1.00 | 35.98 | C |
| ATOM | 21640 | CD2 | TYR | K | 114 | −16.387 | 62.534 | 89.620 | 1.00 | 52.37 | C |
| ATOM | 21642 | C | TYR | K | 114 | −18.442 | 66.285 | 90.940 | 1.00 | 54.24 | C |
| ATOM | 21643 | O | TYR | K | 114 | −19.109 | 67.196 | 90.436 | 1.00 | 51.82 | O |
| ATOM | 21644 | N | GLU | K | 115 | −17.672 | 66.427 | 92.018 | 1.00 | 63.06 | N |
| ATOM | 21646 | CA | GLU | K | 115 | −17.663 | 67.678 | 92.791 | 1.00 | 64.39 | C |
| ATOM | 21648 | CB | GLU | K | 115 | −18.443 | 67.492 | 94.100 | 1.00 | 69.19 | C |
| ATOM | 21651 | CG | GLU | K | 115 | −18.038 | 68.442 | 95.214 | 1.00 | 78.50 | C |
| ATOM | 21654 | CD | GLU | K | 115 | −19.222 | 69.140 | 95.854 | 1.00 | 77.83 | C |
| ATOM | 21655 | OE1 | GLU | K | 115 | −20.158 | 69.570 | 95.126 | 1.00 | 86.06 | O |
| ATOM | 21656 | OE2 | GLU | K | 115 | −19.163 | 69.279 | 97.094 | 1.00 | 57.77 | O |
| ATOM | 21657 | C | GLU | K | 115 | −16.242 | 68.106 | 93.106 | 1.00 | 55.86 | C |
| ATOM | 21658 | O | GLU | K | 115 | −15.512 | 67.391 | 93.797 | 1.00 | 64.32 | O |
| ATOM | 21659 | N | MET | K | 116 | −15.869 | 69.276 | 92.604 | 1.00 | 52.95 | N |
| ATOM | 21661 | CA | MET | K | 116 | −14.463 | 69.716 | 92.524 | 1.00 | 49.85 | C |
| ATOM | 21663 | CB | MET | K | 116 | −14.114 | 70.019 | 91.063 | 1.00 | 49.59 | C |
| ATOM | 21666 | CG | MET | K | 116 | −12.680 | 70.357 | 90.849 | 1.00 | 49.79 | C |
| ATOM | 21669 | SD | MET | K | 116 | −12.258 | 70.763 | 89.150 | 1.00 | 60.41 | S |
| ATOM | 21670 | CE | MET | K | 116 | −13.032 | 72.334 | 88.899 | 1.00 | 68.76 | C |
| ATOM | 21674 | C | MET | K | 116 | −14.209 | 70.973 | 93.364 | 1.00 | 49.28 | C |
| ATOM | 21675 | O | MET | K | 116 | −14.971 | 71.962 | 93.354 | 1.00 | 36.32 | O |
| ATOM | 21676 | N | LYS | K | 117 | −13.140 | 70.964 | 94.140 | 1.00 | 52.96 | N |
| ATOM | 21678 | CA | LYS | K | 117 | −12.968 | 72.103 | 95.035 | 1.00 | 57.54 | C |
| ATOM | 21680 | CB | LYS | K | 117 | −12.154 | 71.717 | 96.270 | 1.00 | 58.64 | C |
| ATOM | 21683 | CG | LYS | K | 117 | −12.523 | 70.313 | 96.852 | 1.00 | 84.46 | C |
| ATOM | 21686 | CD | LYS | K | 117 | −11.329 | 69.491 | 97.411 | 1.00 | 87.86 | C |
| ATOM | 21689 | CE | LYS | K | 117 | −11.684 | 68.730 | 98.705 | 1.00 | 85.61 | C |
| ATOM | 21692 | NZ | LYS | K | 117 | −10.629 | 68.846 | 99.759 | 1.00 | 73.76 | N |
| ATOM | 21696 | C | LYS | K | 117 | −12.364 | 73.218 | 94.186 | 1.00 | 57.28 | C |
| ATOM | 21697 | O | LYS | K | 117 | −11.647 | 72.949 | 93.226 | 1.00 | 53.92 | O |
| ATOM | 21698 | N | LEU | K | 118 | −12.797 | 74.448 | 94.401 | 1.00 | 57.04 | N |
| ATOM | 21700 | CA | LEU | K | 118 | −12.329 | 75.542 | 93.567 | 1.00 | 58.87 | C |
| ATOM | 21702 | CB | LEU | K | 118 | −13.495 | 76.404 | 93.123 | 1.00 | 55.20 | C |
| ATOM | 21705 | CG | LEU | K | 118 | −14.390 | 75.994 | 91.966 | 1.00 | 57.25 | C |
| ATOM | 21707 | CD1 | LEU | K | 118 | −15.396 | 77.122 | 91.773 | 1.00 | 59.47 | C |
| ATOM | 21711 | CD2 | LEU | K | 118 | −13.647 | 75.818 | 90.671 | 1.00 | 68.03 | C |
| ATOM | 21715 | C | LEU | K | 118 | −11.406 | 76.443 | 94.377 | 1.00 | 65.27 | C |
| ATOM | 21716 | O | LEU | K | 118 | −11.860 | 77.079 | 95.332 | 1.00 | 78.24 | O |
| ATOM | 21717 | N | MET | K | 119 | −10.161 | 76.591 | 93.923 | 1.00 | 66.74 | N |
| ATOM | 21719 | CA | MET | K | 119 | −9.150 | 77.439 | 94.569 | 1.00 | 58.80 | C |
| ATOM | 21721 | CB | MET | K | 119 | −7.746 | 76.976 | 94.187 | 1.00 | 51.56 | C |
| ATOM | 21724 | CG | MET | K | 119 | −7.524 | 76.846 | 92.705 | 1.00 | 53.74 | C |
| ATOM | 21727 | SD | MET | K | 119 | −5.917 | 76.165 | 92.404 | 1.00 | 76.99 | S |
| ATOM | 21728 | CE | MET | K | 119 | −5.973 | 74.571 | 93.470 | 1.00 | 79.32 | C |
| ATOM | 21732 | C | MET | K | 119 | −9.241 | 78.955 | 94.344 | 1.00 | 56.91 | C |
| ATOM | 21733 | O | MET | K | 119 | −9.958 | 79.462 | 93.489 | 1.00 | 58.92 | O |
| ATOM | 21734 | N | ASP | K | 120 | −8.457 | 79.670 | 95.134 | 1.00 | 62.99 | N |
| ATOM | 21736 | CA | ASP | K | 120 | −8.443 | 81.114 | 95.129 | 1.00 | 72.25 | C |
| ATOM | 21738 | CB | ASP | K | 120 | −8.464 | 81.568 | 96.581 | 1.00 | 80.28 | C |
| ATOM | 21741 | CG | ASP | K | 120 | −9.637 | 82.460 | 96.853 | 1.00 | 94.50 | C |
| ATOM | 21742 | OD1 | ASP | K | 120 | −9.581 | 83.637 | 96.415 | 1.00 | 100.59 | O |
| ATOM | 21743 | OD2 | ASP | K | 120 | −10.680 | 82.007 | 97.372 | 1.00 | 86.53 | O |
| ATOM | 21744 | C | ASP | K | 120 | −7.212 | 81.682 | 94.439 | 1.00 | 71.78 | C |
| ATOM | 21745 | O | ASP | K | 120 | −6.089 | 81.307 | 94.784 | 1.00 | 65.95 | O |
| ATOM | 21746 | N | LEU | K | 121 | −7.406 | 82.566 | 93.461 | 1.00 | 74.78 | N |
| ATOM | 21748 | CA | LEU | K | 121 | −6.311 | 82.925 | 92.550 | 1.00 | 75.81 | C |
| ATOM | 21750 | CB | LEU | K | 121 | −6.527 | 82.255 | 91.205 | 1.00 | 75.79 | C |
| ATOM | 21753 | CG | LEU | K | 121 | −5.808 | 80.939 | 90.991 | 1.00 | 75.05 | C |
| ATOM | 21755 | CD1 | LEU | K | 121 | −5.806 | 80.716 | 89.484 | 1.00 | 71.57 | C |
| ATOM | 21759 | CD2 | LEU | K | 121 | −4.416 | 81.068 | 91.598 | 1.00 | 84.62 | C |
| ATOM | 21763 | C | LEU | K | 121 | −6.330 | 84.400 | 92.258 | 1.00 | 80.87 | C |
| ATOM | 21764 | O | LEU | K | 121 | −7.396 | 84.932 | 91.907 | 1.00 | 78.62 | O |
| ATOM | 21765 | N | ASP | K | 122 | −5.165 | 85.043 | 92.326 | 1.00 | 86.16 | N |
| ATOM | 21767 | CA | ASP | K | 122 | −5.135 | 86.491 | 92.103 | 1.00 | 92.36 | C |
| ATOM | 21769 | CB | ASP | K | 122 | −4.081 | 87.239 | 92.949 | 1.00 | 92.69 | C |
| ATOM | 21772 | CG | ASP | K | 122 | −2.818 | 86.413 | 93.207 | 1.00 | 100.16 | C |
| ATOM | 21773 | OD1 | ASP | K | 122 | −2.424 | 85.601 | 92.328 | 1.00 | 103.64 | O |
| ATOM | 21774 | OD2 | ASP | K | 122 | −2.139 | 86.549 | 94.255 | 1.00 | 76.14 | O |
| ATOM | 21775 | C | ASP | K | 122 | −5.134 | 86.865 | 90.604 | 1.00 | 92.70 | C |
| ATOM | 21776 | O | ASP | K | 122 | −6.144 | 87.391 | 90.117 | 1.00 | 97.32 | O |
| ATOM | 21777 | N | VAL | K | 123 | −4.074 | 86.575 | 89.850 | 1.00 | 85.39 | N |
| ATOM | 21779 | CA | VAL | K | 123 | −4.108 | 86.823 | 88.398 | 1.00 | 86.72 | C |
| ATOM | 21781 | CB | VAL | K | 123 | −5.068 | 85.822 | 87.658 | 1.00 | 90.15 | C |
| ATOM | 21783 | CG1 | VAL | K | 123 | −4.994 | 84.414 | 88.256 | 1.00 | 88.73 | C |
| ATOM | 21787 | CG2 | VAL | K | 123 | −6.530 | 86.329 | 87.633 | 1.00 | 83.47 | C |
| ATOM | 21791 | C | VAL | K | 123 | −4.572 | 88.227 | 87.976 | 1.00 | 82.57 | C |
| ATOM | 21792 | O | VAL | K | 123 | −5.767 | 88.514 | 87.971 | 1.00 | 73.60 | O |
| ATOM | 21793 | N | GLU | K | 124 | −3.655 | 89.064 | 87.509 | 1.00 | 82.95 | N |
| ATOM | 21795 | CA | GLU | K | 124 | −4.048 | 90.205 | 86.675 | 1.00 | 87.76 | C |
| ATOM | 21797 | CB | GLU | K | 124 | −3.059 | 91.405 | 86.726 | 1.00 | 90.83 | C |
| ATOM | 21800 | CG | GLU | K | 124 | −1.575 | 91.119 | 87.007 | 1.00 | 110.31 | C |
| ATOM | 21803 | CD | GLU | K | 124 | −0.837 | 90.337 | 85.906 | 1.00 | 136.77 | C |
| ATOM | 21804 | OE1 | GLU | K | 124 | −1.065 | 89.109 | 85.737 | 1.00 | 148.18 | O |
| ATOM | 21805 | OE2 | GLU | K | 124 | 0.027 | 90.930 | 85.216 | 1.00 | 135.28 | O |
| ATOM | 21806 | C | GLU | K | 124 | −4.284 | 89.747 | 85.233 | 1.00 | 84.59 | C |
| ATOM | 21807 | O | GLU | K | 124 | −3.500 | 88.961 | 84.677 | 1.00 | 86.90 | O |
| ATOM | 21808 | N | GLN | K | 125 | −5.362 | 90.251 | 84.637 | 1.00 | 78.37 | N |
| ATOM | 21810 | CA | GLN | K | 125 | −5.719 | 89.925 | 83.257 | 1.00 | 74.93 | C |
| ATOM | 21812 | CB | GLN | K | 125 | −7.243 | 89.911 | 83.137 | 1.00 | 76.19 | C |
| ATOM | 21815 | CG | GLN | K | 125 | −7.791 | 90.413 | 81.808 | 1.00 | 90.76 | C |
| ATOM | 21818 | CD | GLN | K | 125 | −8.746 | 89.430 | 81.150 | 1.00 | 99.29 | C |
| ATOM | 21819 | OE1 | GLN | K | 125 | −9.530 | 89.818 | 80.277 | 1.00 | 80.59 | O |
| ATOM | 21820 | NE2 | GLN | K | 125 | −8.671 | 88.157 | 81.553 | 1.00 | 103.53 | N |
| ATOM | 21823 | C | GLN | K | 125 | −5.089 | 90.879 | 82.233 | 1.00 | 66.74 | C |
| ATOM | 21824 | O | GLN | K | 125 | −5.286 | 92.081 | 82.299 | 1.00 | 70.90 | O |
| ATOM | 21825 | N | LEU | K | 126 | −4.311 | 90.354 | 81.295 | 1.00 | 62.84 | N |
| ATOM | 21827 | CA | LEU | K | 126 | −3.606 | 91.201 | 80.325 | 1.00 | 61.09 | C |
| ATOM | 21829 | CB | LEU | K | 126 | −2.441 | 90.482 | 79.622 | 1.00 | 52.66 | C |
| ATOM | 21832 | CG | LEU | K | 126 | −1.524 | 89.772 | 80.607 | 1.00 | 60.55 | C |
| ATOM | 21834 | CD1 | LEU | K | 126 | −0.446 | 88.974 | 79.904 | 1.00 | 60.28 | C |
| ATOM | 21838 | CD2 | LEU | K | 126 | −0.943 | 90.785 | 81.572 | 1.00 | 78.43 | C |
| ATOM | 21842 | C | LEU | K | 126 | −4.563 | 91.656 | 79.251 | 1.00 | 57.38 | C |
| ATOM | 21843 | O | LEU | K | 126 | −5.559 | 90.997 | 78.970 | 1.00 | 62.52 | O |
| ATOM | 21844 | N | GLY | K | 127 | −4.216 | 92.746 | 78.589 | 1.00 | 51.14 | N |
| ATOM | 21846 | CA | GLY | K | 127 | −5.054 | 93.226 | 77.523 | 1.00 | 49.20 | C |
| ATOM | 21849 | C | GLY | K | 127 | −4.408 | 92.879 | 76.217 | 1.00 | 48.33 | C |
| ATOM | 21850 | O | GLY | K | 127 | −3.243 | 93.217 | 76.044 | 1.00 | 55.00 | O |
| ATOM | 21851 | N | ILE | K | 128 | −5.145 | 92.227 | 75.316 | 1.00 | 56.98 | N |
| ATOM | 21853 | CA | ILE | K | 128 | −4.641 | 91.969 | 73.960 | 1.00 | 61.92 | C |
| ATOM | 21855 | CB | ILE | K | 128 | −5.042 | 90.547 | 73.418 | 1.00 | 62.35 | C |
| ATOM | 21857 | CG1 | ILE | K | 128 | −4.991 | 89.420 | 74.480 | 1.00 | 52.00 | C |
| ATOM | 21860 | CD1 | ILE | K | 128 | −3.662 | 89.141 | 75.152 | 1.00 | 56.28 | C |
| ATOM | 21864 | CG2 | ILE | K | 128 | −4.210 | 90.201 | 72.173 | 1.00 | 53.08 | C |
| ATOM | 21868 | C | ILE | K | 128 | −5.051 | 93.038 | 72.930 | 1.00 | 63.33 | C |
| ATOM | 21869 | O | ILE | K | 128 | −6.191 | 93.034 | 72.483 | 1.00 | 56.16 | O |
| ATOM | 21870 | N | PRO | K | 129 | −4.112 | 93.888 | 72.504 | 1.00 | 71.11 | N |
| ATOM | 21871 | CA | PRO | K | 129 | −4.313 | 94.790 | 71.365 | 1.00 | 79.42 | C |
| ATOM | 21873 | CB | PRO | K | 129 | −2.887 | 95.063 | 70.874 | 1.00 | 79.79 | C |
| ATOM | 21876 | CG | PRO | K | 129 | −2.036 | 94.959 | 72.086 | 1.00 | 75.55 | C |
| ATOM | 21879 | CD | PRO | K | 129 | −2.762 | 94.057 | 73.068 | 1.00 | 71.86 | C |
| ATOM | 21882 | C | PRO | K | 129 | −5.109 | 94.173 | 70.235 | 1.00 | 84.78 | C |
| ATOM | 21883 | O | PRO | K | 129 | −4.871 | 93.008 | 69.918 | 1.00 | 91.11 | O |
| ATOM | 21884 | N | GLU | K | 130 | −6.011 | 94.949 | 69.640 | 1.00 | 90.32 | N |
| ATOM | 21886 | CA | GLU | K | 130 | −6.258 | 94.879 | 68.206 | 1.00 | 94.67 | C |
| ATOM | 21888 | CB | GLU | K | 130 | −7.261 | 95.950 | 67.730 | 1.00 | 103.02 | C |
| ATOM | 21891 | CG | GLU | K | 130 | −8.350 | 95.362 | 66.840 | 1.00 | 101.70 | C |
| ATOM | 21894 | CD | GLU | K | 130 | −8.422 | 93.855 | 67.024 | 1.00 | 98.22 | C |
| ATOM | 21895 | OE1 | GLU | K | 130 | −7.553 | 93.111 | 66.506 | 1.00 | 89.00 | O |
| ATOM | 21896 | OE2 | GLU | K | 130 | −9.311 | 93.422 | 67.778 | 1.00 | 90.88 | O |
| ATOM | 21897 | C | GLU | K | 130 | −4.951 | 94.983 | 67.431 | 1.00 | 93.01 | C |
| ATOM | 21898 | O | GLU | K | 130 | −4.191 | 95.951 | 67.592 | 1.00 | 84.87 | O |
| ATOM | 21899 | N | GLN | K | 131 | −4.691 | 93.915 | 66.671 | 1.00 | 94.05 | N |
| ATOM | 21901 | CA | GLN | K | 131 | −3.779 | 93.907 | 65.525 | 1.00 | 93.06 | C |
| ATOM | 21903 | CB | GLN | K | 131 | −2.432 | 93.257 | 65.882 | 1.00 | 92.34 | C |
| ATOM | 21906 | CG | GLN | K | 131 | −2.303 | 92.811 | 67.336 | 1.00 | 101.66 | C |
| ATOM | 21909 | CD | GLN | K | 131 | −0.908 | 92.304 | 67.657 | 1.00 | 117.26 | C |
| ATOM | 21910 | OE1 | GLN | K | 131 | −0.016 | 92.426 | 66.817 | 1.00 | 112.44 | O |
| ATOM | 21911 | NE2 | GLN | K | 131 | −0.711 | 91.740 | 68.858 | 1.00 | 115.22 | N |
| ATOM | 21914 | C | GLN | K | 131 | −4.387 | 93.208 | 64.297 | 1.00 | 91.01 | C |
| ATOM | 21915 | O | GLN | K | 131 | −5.215 | 92.278 | 64.408 | 1.00 | 77.50 | O |
| ATOM | 21916 | N | GLU | K | 132 | −3.979 | 93.708 | 63.128 | 1.00 | 91.44 | N |
| ATOM | 21918 | CA | GLU | K | 132 | −3.757 | 92.875 | 61.950 | 1.00 | 89.48 | C |
| ATOM | 21920 | CB | GLU | K | 132 | −4.199 | 93.570 | 60.658 | 1.00 | 89.18 | C |
| ATOM | 21923 | CG | GLU | K | 132 | −4.100 | 92.667 | 59.433 | 1.00 | 98.70 | C |
| ATOM | 21926 | CD | GLU | K | 132 | −5.245 | 92.886 | 58.465 | 1.00 | 109.96 | C |
| ATOM | 21927 | OE1 | GLU | K | 132 | −5.365 | 94.010 | 57.936 | 1.00 | 117.82 | O |
| ATOM | 21928 | OE2 | GLU | K | 132 | −6.031 | 91.947 | 58.235 | 1.00 | 105.63 | O |
| ATOM | 21929 | C | GLU | K | 132 | −2.299 | 92.426 | 61.852 | 1.00 | 84.28 | C |
| ATOM | 21930 | O | GLU | K | 132 | −1.387 | 93.073 | 62.377 | 1.00 | 76.10 | O |
| ATOM | 21931 | N | TYR | K | 133 | −2.126 | 91.253 | 61.248 | 1.00 | 78.13 | N |
| ATOM | 21933 | CA | TYR | K | 133 | −0.874 | 90.513 | 61.284 | 1.00 | 68.71 | C |
| ATOM | 21935 | CB | TYR | K | 133 | −1.114 | 89.094 | 61.815 | 1.00 | 61.70 | C |
| ATOM | 21938 | CG | TYR | K | 133 | −1.378 | 89.052 | 63.300 | 1.00 | 48.87 | C |
| ATOM | 21939 | CD1 | TYR | K | 133 | −2.665 | 89.198 | 63.806 | 1.00 | 54.79 | C |
| ATOM | 21941 | CE1 | TYR | K | 133 | −2.918 | 89.187 | 65.191 | 1.00 | 37.72 | C |
| ATOM | 21943 | CZ | TYR | K | 133 | −1.868 | 89.031 | 66.089 | 1.00 | 50.48 | C |
| ATOM | 21944 | OH | TYR | K | 133 | −2.097 | 89.008 | 67.456 | 1.00 | 57.12 | O |
| ATOM | 21946 | CE2 | TYR | K | 133 | −0.585 | 88.863 | 65.605 | 1.00 | 50.91 | C |
| ATOM | 21948 | CD2 | TYR | K | 133 | −0.342 | 88.871 | 64.215 | 1.00 | 58.08 | C |
| ATOM | 21950 | C | TYR | K | 133 | −0.373 | 90.499 | 59.844 | 1.00 | 67.85 | C |
| ATOM | 21951 | O | TYR | K | 133 | −1.160 | 90.384 | 58.900 | 1.00 | 74.46 | O |
| ATOM | 21952 | N | SER | K | 134 | 0.923 | 90.689 | 59.655 | 1.00 | 61.98 | N |
| ATOM | 21954 | CA | SER | K | 134 | 1.424 | 90.883 | 58.304 | 1.00 | 66.06 | C |
| ATOM | 21956 | CB | SER | K | 134 | 2.736 | 91.696 | 58.324 | 1.00 | 67.33 | C |
| ATOM | 21959 | OG | SER | K | 134 | 3.804 | 90.983 | 58.926 | 1.00 | 52.45 | O |
| ATOM | 21961 | C | SER | K | 134 | 1.550 | 89.573 | 57.497 | 1.00 | 69.43 | C |
| ATOM | 21962 | O | SER | K | 134 | 2.301 | 89.518 | 56.527 | 1.00 | 70.52 | O |
| ATOM | 21963 | N | CYS | K | 135 | 0.802 | 88.539 | 57.885 | 1.00 | 71.55 | N |
| ATOM | 21965 | CA | CYS | K | 135 | 0.917 | 87.184 | 57.336 | 1.00 | 68.24 | C |
| ATOM | 21967 | CB | CYS | K | 135 | 2.362 | 86.714 | 57.257 | 1.00 | 69.36 | C |
| ATOM | 21970 | SG | CYS | K | 135 | 2.417 | 84.928 | 57.016 | 1.00 | 113.13 | S |
| ATOM | 21971 | C | CYS | K | 135 | 0.149 | 86.193 | 58.206 | 1.00 | 59.51 | C |
| ATOM | 21972 | O | CYS | K | 135 | 0.345 | 86.143 | 59.423 | 1.00 | 56.58 | O |
| ATOM | 21973 | N | VAL | K | 136 | −0.768 | 85.454 | 57.583 | 1.00 | 51.41 | N |
| ATOM | 21975 | CA | VAL | K | 136 | −1.623 | 84.524 | 58.301 | 1.00 | 44.14 | C |
| ATOM | 21977 | CB | VAL | K | 136 | −3.044 | 85.098 | 58.539 | 1.00 | 44.49 | C |
| ATOM | 21979 | CG1 | VAL | K | 136 | −4.042 | 84.020 | 58.994 | 1.00 | 37.15 | C |
| ATOM | 21983 | CG2 | VAL | K | 136 | −3.002 | 86.235 | 59.555 | 1.00 | 56.88 | C |
| ATOM | 21987 | C | VAL | K | 136 | −1.695 | 83.193 | 57.565 | 1.00 | 43.73 | C |
| ATOM | 21988 | O | VAL | K | 136 | −2.269 | 83.044 | 56.497 | 1.00 | 49.95 | O |
| ATOM | 21989 | N | VAL | K | 137 | −1.149 | 82.171 | 58.183 | 1.00 | 43.06 | N |
| ATOM | 21991 | CA | VAL | K | 137 | −1.176 | 80.876 | 57.568 | 1.00 | 42.47 | C |
| ATOM | 21993 | CB | VAL | K | 137 | 0.113 | 80.201 | 57.910 | 1.00 | 35.37 | C |
| ATOM | 21995 | CG1 | VAL | K | 137 | 0.025 | 78.810 | 57.409 | 1.00 | 54.34 | C |
| ATOM | 21999 | CG2 | VAL | K | 137 | 1.264 | 80.938 | 57.235 | 1.00 | 49.61 | C |
| ATOM | 22003 | C | VAL | K | 137 | −2.336 | 80.053 | 58.125 | 1.00 | 46.80 | C |
| ATOM | 22004 | O | VAL | K | 137 | −2.424 | 79.839 | 59.339 | 1.00 | 44.46 | O |
| ATOM | 22005 | N | LYS | K | 138 | −3.209 | 79.569 | 57.248 | 1.00 | 45.36 | N |
| ATOM | 22007 | CA | LYS | K | 138 | −4.257 | 78.630 | 57.647 | 1.00 | 48.34 | C |
| ATOM | 22009 | CB | LYS | K | 138 | −5.557 | 78.934 | 56.910 | 1.00 | 56.74 | C |
| ATOM | 22012 | CG | LYS | K | 138 | −6.890 | 78.565 | 57.583 | 1.00 | 65.82 | C |
| ATOM | 22015 | CD | LYS | K | 138 | −7.972 | 79.631 | 57.268 | 1.00 | 75.35 | C |
| ATOM | 22018 | CE | LYS | K | 138 | −9.410 | 79.145 | 57.545 | 1.00 | 90.19 | C |
| ATOM | 22021 | NZ | LYS | K | 138 | −9.787 | 77.806 | 56.963 | 1.00 | 72.85 | N |
| ATOM | 22025 | C | LYS | K | 138 | −3.766 | 77.319 | 57.107 | 1.00 | 49.76 | C |
| ATOM | 22026 | O | LYS | K | 138 | −3.459 | 77.255 | 55.925 | 1.00 | 57.27 | O |
| ATOM | 22027 | N | MET | K | 139 | −3.663 | 76.295 | 57.947 | 1.00 | 45.73 | N |
| ATOM | 22029 | CA | MET | K | 139 | −3.177 | 74.998 | 57.485 | 1.00 | 46.18 | C |
| ATOM | 22031 | CB | MET | K | 139 | −1.654 | 74.908 | 57.542 | 1.00 | 45.08 | C |
| ATOM | 22034 | CG | MET | K | 139 | −1.150 | 74.910 | 58.952 | 1.00 | 38.10 | C |
| ATOM | 22037 | SD | MET | K | 139 | 0.545 | 74.357 | 59.063 | 1.00 | 48.50 | S |
| ATOM | 22038 | CE | MET | K | 139 | 1.291 | 75.835 | 58.707 | 1.00 | 60.66 | C |
| ATOM | 22042 | C | MET | K | 139 | −3.753 | 73.884 | 58.337 | 1.00 | 42.68 | C |
| ATOM | 22043 | O | MET | K | 139 | −4.501 | 74.110 | 59.272 | 1.00 | 50.02 | O |
| ATOM | 22044 | N | PRO | K | 140 | −3.527 | 72.654 | 57.914 | 1.00 | 40.49 | N |
| ATOM | 22045 | CA | PRO | K | 140 | −4.047 | 71.506 | 50.668 | 1.00 | 33.11 | C |
| ATOM | 22047 | CB | PRO | K | 140 | −3.666 | 70.317 | 57.798 | 1.00 | 28.87 | C |
| ATOM | 22050 | CG | PRO | K | 140 | −3.482 | 70.892 | 56.372 | 1.00 | 24.92 | C |
| ATOM | 22053 | CD | PRO | K | 140 | −2.972 | 72.297 | 56.587 | 1.00 | 37.23 | C |
| ATOM | 22056 | C | PRO | K | 140 | −3.407 | 71.401 | 60.040 | 1.00 | 45.64 | C |
| ATOM | 22057 | O | PRO | K | 140 | −2.241 | 71.785 | 60.228 | 1.00 | 56.19 | O |
| ATOM | 22058 | N | SER | K | 141 | −4.161 | 70.898 | 61.007 | 1.00 | 40.18 | N |
| ATOM | 22060 | CA | SER | K | 141 | −3.731 | 71.062 | 62.365 | 1.00 | 35.69 | C |
| ATOM | 22062 | CB | SER | K | 141 | −4.949 | 71.202 | 63.244 | 1.00 | 40.88 | C |
| ATOM | 22065 | OG | SER | K | 141 | −5.756 | 70.074 | 63.088 | 1.00 | 30.43 | O |
| ATOM | 22067 | C | SER | K | 141 | −2.876 | 69.858 | 62.744 | 1.00 | 42.50 | C |
| ATOM | 22068 | O | SER | K | 141 | −2.030 | 69.911 | 63.647 | 1.00 | 41.73 | O |
| ATOM | 22069 | N | GLY | K | 142 | −3.064 | 68.774 | 62.002 | 1.00 | 47.78 | N |
| ATOM | 22071 | CA | GLY | K | 142 | −2.204 | 67.604 | 62.117 | 1.00 | 45.25 | C |
| ATOM | 22074 | C | GLY | K | 142 | −0.836 | 67.814 | 61.486 | 1.00 | 49.74 | C |
| ATOM | 22075 | O | GLY | K | 142 | 0.161 | 67.219 | 61.936 | 1.00 | 43.27 | O |
| ATOM | 22076 | N | GLU | K | 143 | −0.781 | 68.656 | 60.453 | 1.00 | 47.16 | N |
| ATOM | 22078 | CA | GLU | K | 143 | 0.468 | 68.812 | 59.743 | 1.00 | 49.22 | C |
| ATOM | 22080 | CB | GLU | K | 143 | 0.324 | 69.413 | 58.346 | 1.00 | 50.92 | C |
| ATOM | 22083 | CG | GLU | K | 143 | 1.639 | 69.346 | 57.579 | 1.00 | 51.73 | C |
| ATOM | 22086 | CD | GLU | K | 143 | 2.144 | 67.926 | 57.363 | 1.00 | 64.45 | C |
| ATOM | 22087 | OE1 | GLU | K | 143 | 1.320 | 67.063 | 56.990 | 1.00 | 76.49 | O |
| ATOM | 22088 | OE2 | GLU | K | 143 | 3.357 | 67.657 | 57.545 | 1.00 | 48.37 | O |
| ATOM | 22089 | C | GLU | K | 143 | 1.342 | 69.686 | 60.602 | 1.00 | 47.27 | C |
| ATOM | 22090 | O | GLU | K | 143 | 2.474 | 69.301 | 60.889 | 1.00 | 44.48 | O |
| ATOM | 22091 | N | PHE | K | 144 | 0.793 | 70.819 | 61.038 | 1.00 | 45.09 | N |
| ATOM | 22093 | CA | PHE | K | 144 | 1.418 | 71.630 | 62.085 | 1.00 | 41.56 | C |
| ATOM | 22095 | CB | PHE | K | 144 | 0.461 | 72.655 | 62.681 | 1.00 | 40.45 | C |
| ATOM | 22098 | CG | PHE | K | 144 | 1.165 | 73.700 | 63.455 | 1.00 | 42.16 | C |
| ATOM | 22099 | CD1 | PHE | K | 144 | 2.181 | 74.428 | 62.863 | 1.00 | 49.39 | C |
| ATOM | 22101 | CE1 | PHE | K | 144 | 2.878 | 75.359 | 63.573 | 1.00 | 42.83 | C |
| ATOM | 22103 | CZ | PHE | K | 144 | 2.552 | 75.587 | 64.894 | 1.00 | 48.75 | C |
| ATOM | 22105 | CE2 | PHE | K | 144 | 1.571 | 74.845 | 65.505 | 1.00 | 35.70 | C |
| ATOM | 22107 | CD2 | PHE | K | 144 | 0.890 | 73.891 | 64.785 | 1.00 | 51.47 | C |
| ATOM | 22109 | C | PHE | K | 144 | 1.938 | 70.824 | 63.264 | 1.00 | 36.23 | C |
| ATOM | 22110 | O | PHE | K | 144 | 2.980 | 71.118 | 63.841 | 1.00 | 33.43 | O |
| ATOM | 22111 | N | ALA | K | 145 | 1.204 | 69.806 | 63.663 | 1.00 | 34.17 | N |
| ATOM | 22113 | CA | ALA | K | 145 | 1.688 | 69.070 | 64.809 | 1.00 | 38.10 | C |
| ATOM | 22115 | CB | ALA | K | 145 | 0.604 | 68.256 | 65.447 | 1.00 | 41.74 | C |
| ATOM | 22119 | C | ALA | K | 145 | 2.825 | 68.194 | 64.390 | 1.00 | 36.00 | C |
| ATOM | 22120 | O | ALA | K | 145 | 3.745 | 67.981 | 65.174 | 1.00 | 39.16 | O |
| ATOM | 22121 | N | ARG | K | 146 | 2.694 | 67.612 | 63.205 | 1.00 | 42.73 | N |
| ATOM | 22123 | CA | ARG | K | 146 | 3.695 | 66.687 | 62.677 | 1.00 | 44.73 | C |
| ATOM | 22125 | CB | ARG | K | 146 | 3.264 | 66.275 | 61.256 | 1.00 | 51.02 | C |
| ATOM | 22128 | CG | ARG | K | 146 | 3.801 | 64.984 | 60.635 | 1.00 | 52.04 | C |
| ATOM | 22131 | CD | ARG | K | 146 | 2.875 | 64.414 | 59.538 | 1.00 | 66.97 | C |
| ATOM | 22134 | NE | ARG | K | 146 | 1.508 | 64.063 | 59.975 | 1.00 | 72.42 | N |
| ATOM | 22136 | CZ | ARG | K | 146 | 0.385 | 64.590 | 59.468 | 1.00 | 74.28 | C |
| ATOM | 22137 | NH1 | ARG | K | 146 | 0.469 | 65.525 | 58.529 | 1.00 | 68.83 | N |
| ATOM | 22140 | NH2 | ARG | K | 146 | −0.823 | 64.193 | 59.878 | 1.00 | 71.54 | N |
| ATOM | 22143 | C | ARG | K | 146 | 5.011 | 67.486 | 62.673 | 1.00 | 44.55 | C |
| ATOM | 22144 | O | ARG | K | 146 | 6.094 | 66.987 | 63.025 | 1.00 | 43.00 | O |
| ATOM | 22145 | N | ILE | K | 147 | 4.899 | 68.750 | 62.283 | 1.00 | 39.23 | N |
| ATOM | 22147 | CA | ILE | K | 147 | 6.081 | 69.481 | 61.872 | 1.00 | 39.73 | C |
| ATOM | 22149 | CB | ILE | K | 147 | 5.685 | 70.671 | 61.002 | 1.00 | 34.79 | C |
| ATOM | 22151 | CG1 | ILE | K | 147 | 5.615 | 70.223 | 59.542 | 1.00 | 24.30 | C |
| ATOM | 22154 | CD1 | ILE | K | 147 | 5.047 | 71.304 | 58.605 | 1.00 | 49.00 | C |
| ATOM | 22158 | CG2 | ILE | K | 147 | 6.617 | 71.838 | 61.300 | 1.00 | 28.63 | C |
| ATOM | 22162 | C | ILE | K | 147 | 6.754 | 69.977 | 63.142 | 1.00 | 45.59 | C |
| ATOM | 22163 | O | ILE | K | 147 | 7.984 | 70.023 | 63.227 | 1.00 | 50.08 | O |
| ATOM | 22164 | N | CYS | K | 148 | 5.936 | 70.354 | 64.123 | 1.00 | 40.49 | N |
| ATOM | 22166 | CA | CYS | K | 148 | 6.485 | 70.884 | 65.357 | 1.00 | 41.02 | C |
| ATOM | 22168 | CB | CYS | K | 148 | 5.420 | 71.600 | 66.181 | 1.00 | 38.44 | C |
| ATOM | 22171 | SG | CYS | K | 148 | 5.013 | 73.262 | 65.558 | 1.00 | 41.67 | S |
| ATOM | 22172 | C | CYS | K | 148 | 7.136 | 69.744 | 66.114 | 1.00 | 41.86 | C |
| ATOM | 22173 | O | CYS | K | 148 | 8.252 | 69.866 | 66.585 | 1.00 | 43.59 | O |
| ATOM | 22174 | N | ARG | K | 149 | 6.485 | 68.593 | 66.157 | 1.00 | 44.05 | N |
| ATOM | 22176 | CA | ARG | K | 149 | 7.104 | 67.410 | 66.762 | 1.00 | 44.50 | C |
| ATOM | 22178 | CB | ARG | K | 149 | 6.124 | 66.238 | 66.695 | 1.00 | 42.75 | C |
| ATOM | 22181 | CG | ARG | K | 149 | 6.557 | 64.994 | 67.414 | 1.00 | 53.77 | C |
| ATOM | 22184 | CD | ARG | K | 149 | 5.823 | 63.722 | 66.961 | 1.00 | 77.55 | C |
| ATOM | 22187 | NE | ARG | K | 149 | 6.350 | 63.200 | 65.702 | 1.00 | 85.37 | N |
| ATOM | 22189 | CZ | ARG | K | 149 | 5.615 | 62.959 | 64.625 | 1.00 | 85.08 | C |
| ATOM | 22190 | NH1 | ARG | K | 149 | 4.301 | 63.147 | 64.650 | 1.00 | 90.90 | N |
| ATOM | 22193 | NH2 | ARG | K | 149 | 6.201 | 62.538 | 63.514 | 1.00 | 69.04 | N |
| ATOM | 22196 | C | ARG | K | 149 | 8.408 | 67.040 | 66.049 | 1.00 | 42.07 | C |
| ATOM | 22197 | O | ARG | K | 149 | 9.462 | 66.870 | 66.669 | 1.00 | 39.45 | O |
| ATOM | 22198 | N | ASP | K | 150 | 8.315 | 66.870 | 64.738 | 1.00 | 44.27 | N |
| ATOM | 22200 | CA | ASP | K | 150 | 9.452 | 66.441 | 63.962 | 1.00 | 43.70 | C |
| ATOM | 22202 | CB | ASP | K | 150 | 9.024 | 66.297 | 62.520 | 1.00 | 48.81 | C |
| ATOM | 22205 | CG | ASP | K | 150 | 8.254 | 65.038 | 62.303 | 1.00 | 50.69 | C |
| ATOM | 22206 | OD1 | ASP | K | 150 | 8.120 | 64.336 | 63.319 | 1.00 | 65.85 | O |
| ATOM | 22207 | OD2 | ASP | K | 150 | 7.772 | 64.659 | 61.212 | 1.00 | 55.92 | O |
| ATOM | 22208 | C | ASP | K | 150 | 10.645 | 67.381 | 64.091 | 1.00 | 46.33 | C |
| ATOM | 22209 | O | ASP | K | 150 | 11.757 | 66.930 | 64.382 | 1.00 | 45.18 | O |
| ATOM | 22210 | N | LEU | K | 151 | 10.425 | 68.687 | 63.994 | 1.00 | 42.80 | N |
| ATOM | 22212 | CA | LEU | K | 151 | 11.571 | 69.597 | 64.091 | 1.00 | 43.05 | C |
| ATOM | 22214 | CB | LEU | K | 151 | 11.228 | 71.014 | 63.647 | 1.00 | 44.01 | C |
| ATOM | 22217 | CG | LEU | K | 151 | 10.989 | 71.026 | 62.134 | 1.00 | 39.86 | C |
| ATOM | 22219 | CD1 | LEU | K | 151 | 10.626 | 72.364 | 61.648 | 1.00 | 32.90 | C |
| ATOM | 22223 | CD2 | LEU | K | 151 | 12.211 | 70.542 | 61.410 | 1.00 | 38.17 | C |
| ATOM | 22227 | C | LEU | K | 151 | 12.167 | 69.644 | 65.466 | 1.00 | 40.63 | C |
| ATOM | 22228 | O | LEU | K | 151 | 13.306 | 70.066 | 65.623 | 1.00 | 51.07 | O |
| ATOM | 22229 | N | SER | K | 152 | 11.425 | 69.171 | 66.456 | 1.00 | 42.35 | N |
| ATOM | 22231 | CA | SER | K | 152 | 11.864 | 69.261 | 67.846 | 1.00 | 45.24 | C |
| ATOM | 22233 | CB | SER | K | 152 | 10.655 | 69.117 | 68.786 | 1.00 | 40.47 | C |
| ATOM | 22236 | OG | SER | K | 152 | 10.381 | 67.762 | 69.113 | 1.00 | 48.03 | O |
| ATOM | 22238 | C | SER | K | 152 | 12.973 | 68.227 | 68.122 | 1.00 | 44.56 | C |
| ATOM | 22239 | O | SER | K | 152 | 13.725 | 68.330 | 69.090 | 1.00 | 45.10 | O |
| ATOM | 22240 | N | HIS | K | 153 | 13.092 | 67.255 | 67.230 | 1.00 | 37.47 | N |
| ATOM | 22242 | CA | HIS | K | 153 | 14.137 | 66.260 | 67.318 | 1.00 | 46.17 | C |
| ATOM | 22244 | CB | HIS | K | 153 | 13.653 | 65.020 | 66.585 | 1.00 | 55.23 | C |
| ATOM | 22247 | CG | HIS | K | 153 | 12.459 | 64.393 | 67.226 | 1.00 | 65.99 | C |
| ATOM | 22248 | ND1 | HIS | K | 153 | 11.421 | 63.860 | 66.494 | 1.00 | 79.77 | N |
| ATOM | 22250 | CE1 | HIS | K | 153 | 10.502 | 63.396 | 67.321 | 1.00 | 75.16 | C |
| ATOM | 22252 | NE2 | HIS | K | 153 | 10.907 | 63.615 | 68.559 | 1.00 | 79.99 | N |
| ATOM | 22254 | CD2 | HIS | K | 153 | 12.139 | 64.220 | 68.530 | 1.00 | 56.20 | C |
| ATOM | 22256 | C | HIS | K | 153 | 15.434 | 66.736 | 66.689 | 1.00 | 46.55 | C |
| ATOM | 22257 | O | HIS | K | 153 | 16.522 | 66.178 | 66.894 | 1.00 | 55.05 | O |
| ATOM | 22258 | N | ILE | K | 154 | 15.312 | 67.817 | 65.950 | 1.00 | 44.96 | N |
| ATOM | 22260 | CA | ILE | K | 154 | 16.467 | 68.394 | 65.306 | 1.00 | 49.99 | C |
| ATOM | 22262 | CB | ILE | K | 154 | 16.071 | 68.824 | 63.910 | 1.00 | 42.11 | C |
| ATOM | 22264 | CG1 | ILE | K | 154 | 15.301 | 67.703 | 63.235 | 1.00 | 46.27 | C |
| ATOM | 22267 | CD1 | ILE | K | 154 | 16.191 | 66.782 | 62.492 | 1.00 | 51.46 | C |
| ATOM | 22271 | CG2 | ILE | K | 154 | 17.285 | 69.103 | 63.117 | 1.00 | 42.26 | C |
| ATOM | 22275 | C | ILE | K | 154 | 17.005 | 69.589 | 66.104 | 1.00 | 54.64 | C |
| ATOM | 22276 | O | ILE | K | 154 | 18.212 | 69.873 | 66.112 | 1.00 | 48.94 | O |
| ATOM | 22277 | N | GLY | K | 155 | 16.112 | 70.301 | 66.782 | 1.00 | 53.15 | N |
| ATOM | 22279 | CA | GLY | K | 155 | 16.521 | 71.563 | 67.386 | 1.00 | 54.36 | C |
| ATOM | 22282 | C | GLY | K | 155 | 15.487 | 72.063 | 68.371 | 1.00 | 50.66 | C |
| ATOM | 22283 | O | GLY | K | 155 | 14.433 | 71.429 | 68.548 | 1.00 | 46.22 | O |
| ATOM | 22284 | N | ASP | K | 156 | 15.793 | 73.161 | 69.050 | 1.00 | 45.90 | N |
| ATOM | 22286 | CA | ASP | K | 156 | 14.843 | 73.636 | 70.046 | 1.00 | 49.43 | C |
| ATOM | 22288 | CB | ASP | K | 156 | 15.354 | 73.728 | 71.492 | 1.00 | 44.07 | C |
| ATOM | 22291 | CG | ASP | K | 156 | 16.826 | 74.016 | 71.616 | 1.00 | 76.25 | C |
| ATOM | 22292 | OD1 | ASP | K | 156 | 17.448 | 73.264 | 72.409 | 1.00 | 81.05 | O |
| ATOM | 22293 | OD2 | ASP | K | 156 | 17.407 | 74.992 | 71.072 | 1.00 | 101.19 | O |
| ATOM | 22294 | C | ASP | K | 156 | 14.052 | 74.856 | 69.660 | 1.00 | 40.52 | C |
| ATOM | 22295 | O | ASP | K | 156 | 12.924 | 75.052 | 70.080 | 1.00 | 47.07 | O |
| ATOM | 22296 | N | ALA | K | 157 | 14.583 | 75.657 | 68.769 | 1.00 | 46.38 | N |
| ATOM | 22298 | CA | ALA | K | 157 | 13.683 | 76.566 | 68.114 | 1.00 | 41.60 | C |
| ATOM | 22300 | CB | ALA | K | 157 | 14.124 | 77.986 | 68.260 | 1.00 | 41.08 | C |
| ATOM | 22304 | C | ALA | K | 157 | 13.518 | 76.200 | 66.663 | 1.00 | 43.07 | C |
| ATOM | 22305 | O | ALA | K | 157 | 14.250 | 75.389 | 66.080 | 1.00 | 48.00 | O |
| ATOM | 22306 | N | VAL | K | 158 | 12.544 | 76.910 | 66.131 | 1.00 | 40.53 | N |
| ATOM | 22308 | CA | VAL | K | 158 | 12.054 | 76.789 | 64.790 | 1.00 | 42.07 | C |
| ATOM | 22310 | CB | VAL | K | 158 | 10.646 | 76.177 | 64.800 | 1.00 | 35.59 | C |
| ATOM | 22312 | CG1 | VAL | K | 158 | 9.686 | 77.101 | 65.455 | 1.00 | 39.71 | C |
| ATOM | 22316 | CG2 | VAL | K | 158 | 10.161 | 75.934 | 63.421 | 1.00 | 36.49 | C |
| ATOM | 22320 | C | VAL | K | 158 | 12.002 | 78.216 | 64.242 | 1.00 | 42.91 | C |
| ATOM | 22321 | O | VAL | K | 158 | 11.509 | 79.147 | 64.871 | 1.00 | 35.85 | O |
| ATOM | 22322 | N | VAL | K | 159 | 12.462 | 78.359 | 63.013 | 1.00 | 48.31 | N |
| ATOM | 22324 | CA | VAL | K | 159 | 12.209 | 79.569 | 62.258 | 1.00 | 47.44 | C |
| ATOM | 22326 | CB | VAL | K | 159 | 13.448 | 79.977 | 61.493 | 1.00 | 48.72 | C |
| ATOM | 22328 | CG1 | VAL | K | 159 | 13.210 | 81.343 | 60.897 | 1.00 | 56.13 | C |
| ATOM | 22332 | CG2 | VAL | K | 159 | 14.663 | 79.931 | 62.401 | 1.00 | 39.26 | C |
| ATOM | 22336 | C | VAL | K | 159 | 11.099 | 79.421 | 61.239 | 1.00 | 40.96 | C |
| ATOM | 22337 | O | VAL | K | 159 | 11.247 | 78.830 | 60.187 | 1.00 | 48.56 | O |
| ATOM | 22338 | N | ILE | K | 160 | 9.971 | 80.008 | 61.554 | 1.00 | 44.24 | N |
| ATOM | 22340 | CA | ILE | K | 160 | 8.852 | 80.051 | 60.639 | 1.00 | 46.47 | C |
| ATOM | 22342 | CB | ILE | K | 160 | 7.594 | 80.204 | 61.463 | 1.00 | 33.31 | C |
| ATOM | 22344 | CG1 | ILE | K | 160 | 7.390 | 79.033 | 62.397 | 1.00 | 41.25 | C |
| ATOM | 22347 | CD1 | ILE | K | 160 | 6.354 | 79.368 | 63.434 | 1.00 | 60.15 | C |
| ATOM | 22351 | CG2 | ILE | K | 160 | 6.424 | 80.296 | 60.573 | 1.00 | 48.80 | C |
| ATOM | 22355 | C | ILE | K | 160 | 8.977 | 81.262 | 59.716 | 1.00 | 52.16 | C |
| ATOM | 22356 | O | ILE | K | 160 | 9.303 | 82.366 | 60.169 | 1.00 | 53.22 | O |
| ATOM | 22357 | N | SER | K | 161 | 8.662 | 81.076 | 58.438 | 1.00 | 55.13 | N |
| ATOM | 22359 | CA | SER | K | 161 | 9.162 | 81.982 | 57.403 | 1.00 | 56.30 | C |
| ATOM | 22361 | CB | SER | K | 161 | 10.477 | 81.425 | 56.863 | 1.00 | 55.37 | C |
| ATOM | 22364 | OG | SER | K | 161 | 11.461 | 82.431 | 56.860 | 1.00 | 69.35 | O |
| ATOM | 22366 | C | SER | K | 161 | 8.163 | 82.020 | 56.273 | 1.00 | 49.73 | C |
| ATOM | 22367 | O | SER | K | 161 | 8.239 | 81.181 | 55.417 | 1.00 | 42.46 | O |
| ATOM | 22368 | N | CYS | K | 162 | 7.148 | 82.869 | 56.315 | 1.00 | 61.92 | N |
| ATOM | 22370 | CA | CYS | K | 162 | 6.037 | 82.713 | 55.373 | 1.00 | 69.56 | C |
| ATOM | 22372 | CB | CYS | K | 162 | 4.675 | 82.805 | 56.054 | 1.00 | 70.71 | C |
| ATOM | 22375 | SG | CYS | K | 162 | 3.959 | 84.438 | 58.780 | 1.00 | 108.74 | S |
| ATOM | 22376 | C | CYS | K | 162 | 6.107 | 83.745 | 54.249 | 1.00 | 69.42 | C |
| ATOM | 22377 | O | CYS | K | 162 | 6.805 | 84.758 | 54.339 | 1.00 | 61.92 | O |
| ATOM | 22378 | N | ALA | K | 163 | 5.407 | 83.441 | 53.163 | 1.00 | 72.65 | N |
| ATOM | 22380 | CA | ALA | K | 163 | 5.541 | 84.177 | 51.908 | 1.00 | 77.46 | C |
| ATOM | 22382 | CB | ALA | K | 163 | 6.848 | 83.826 | 51.224 | 1.00 | 80.23 | C |
| ATOM | 22386 | C | ALA | K | 163 | 4.351 | 83.842 | 51.002 | 1.00 | 82.05 | C |
| ATOM | 22387 | O | ALA | K | 163 | 3.437 | 83.123 | 51.432 | 1.00 | 88.27 | O |
| ATOM | 22388 | N | LYS | K | 164 | 4.319 | 84.386 | 49.784 | 1.00 | 79.39 | N |
| ATOM | 22390 | CA | LYS | K | 164 | 3.231 | 84.070 | 48.854 | 1.00 | 77.29 | C |
| ATOM | 22392 | CB | LYS | K | 164 | 3.321 | 84.991 | 47.627 | 1.00 | 76.95 | C |
| ATOM | 22395 | CG | LYS | K | 164 | 2.295 | 84.739 | 46.515 | 1.00 | 82.44 | C |
| ATOM | 22398 | CD | LYS | K | 164 | 2.775 | 85.185 | 45.125 | 1.00 | 94.98 | C |
| ATOM | 22401 | CE | LYS | K | 164 | 3.630 | 84.131 | 44.393 | 1.00 | 103.67 | C |
| ATOM | 22404 | NZ | LYS | K | 164 | 4.429 | 84.710 | 43.257 | 1.00 | 106.46 | N |
| ATOM | 22408 | C | LYS | K | 164 | 3.371 | 82.591 | 48.468 | 1.00 | 79.68 | C |
| ATOM | 22409 | O | LYS | K | 164 | 2.374 | 81.876 | 48.293 | 1.00 | 65.26 | O |
| ATOM | 22410 | N | ASP | K | 165 | 4.645 | 82.185 | 48.374 | 1.00 | 88.15 | N |
| ATOM | 22412 | CA | ASP | K | 165 | 5.142 | 80.843 | 48.048 | 1.00 | 89.74 | C |
| ATOM | 22414 | CB | ASP | K | 165 | 6.642 | 80.790 | 48.437 | 1.00 | 92.31 | C |
| ATOM | 22417 | CG | ASP | K | 165 | 7.569 | 80.370 | 47.284 | 1.00 | 101.92 | C |
| ATOM | 22418 | OD1 | ASP | K | 165 | 7.148 | 79.619 | 46.381 | 1.00 | 108.96 | O |
| ATOM | 22419 | OD2 | ASP | K | 165 | 8.774 | 80.695 | 47.214 | 1.00 | 90.64 | O |
| ATOM | 22420 | C | ASP | K | 165 | 4.355 | 79.748 | 48.805 | 1.00 | 90.68 | C |
| ATOM | 22421 | O | ASP | K | 165 | 3.348 | 79.207 | 48.307 | 1.00 | 85.64 | O |
| ATOM | 22422 | N | GLY | K | 166 | 4.839 | 79.417 | 50.005 | 1.00 | 85.53 | N |
| ATOM | 22424 | CA | GLY | K | 166 | 3.994 | 79.167 | 51.167 | 1.00 | 78.07 | C |
| ATOM | 22427 | C | GLY | K | 166 | 4.814 | 79.397 | 52.420 | 1.00 | 67.92 | C |
| ATOM | 22428 | O | GLY | K | 166 | 5.577 | 80.353 | 52.484 | 1.00 | 69.91 | O |
| ATOM | 22429 | N | VAL | K | 167 | 4.698 | 78.525 | 53.409 | 1.00 | 58.70 | N |
| ATOM | 22431 | CA | VAL | K | 167 | 5.381 | 78.763 | 54.677 | 1.00 | 52.24 | C |
| ATOM | 22433 | CB | VAL | K | 167 | 4.404 | 78.540 | 55.847 | 1.00 | 59.03 | C |
| ATOM | 22435 | CG1 | VAL | K | 167 | 3.756 | 77.124 | 55.809 | 1.00 | 51.86 | C |
| ATOM | 22439 | CG2 | VAL | K | 167 | 5.105 | 78.792 | 57.161 | 1.00 | 64.76 | C |
| ATOM | 22443 | C | VAL | K | 167 | 6.583 | 77.821 | 54.801 | 1.00 | 46.83 | C |
| ATOM | 22444 | O | VAL | K | 167 | 6.511 | 76.674 | 54.368 | 1.00 | 35.06 | O |
| ATOM | 22445 | N | LYS | K | 168 | 7.677 | 78.298 | 55.393 | 1.00 | 41.73 | N |
| ATOM | 22447 | CA | LYS | K | 168 | 8.828 | 77.436 | 55.647 | 1.00 | 45.06 | C |
| ATOM | 22449 | CB | LYS | K | 168 | 10.032 | 77.921 | 54.855 | 1.00 | 47.34 | C |
| ATOM | 22452 | CG | LYS | K | 168 | 11.196 | 76.936 | 54.803 | 1.00 | 42.76 | C |
| ATOM | 22455 | CD | LYS | K | 168 | 12.261 | 77.390 | 53.828 | 1.00 | 58.76 | C |
| ATOM | 22458 | CE | LYS | K | 168 | 13.265 | 78.392 | 54.417 | 1.00 | 59.17 | C |
| ATOM | 22461 | NZ | LYS | K | 168 | 14.489 | 78.466 | 53.558 | 1.00 | 57.14 | N |
| ATOM | 22465 | C | LYS | K | 168 | 9.195 | 77.313 | 57.146 | 1.00 | 48.96 | C |
| ATOM | 22466 | O | LYS | K | 168 | 9.139 | 78.288 | 57.897 | 1.00 | 52.07 | O |
| ATOM | 22467 | N | PHE | K | 169 | 9.570 | 76.108 | 57.571 | 1.00 | 41.01 | N |
| ATOM | 22469 | CA | PHE | K | 169 | 9.758 | 75.820 | 58.978 | 1.00 | 44.69 | C |
| ATOM | 22471 | CB | PHE | K | 169 | 8.749 | 74.804 | 59.504 | 1.00 | 44.87 | C |
| ATOM | 22474 | CG | PHE | K | 169 | 7.358 | 75.297 | 59.525 | 1.00 | 41.10 | C |
| ATOM | 22475 | CD1 | PHE | K | 169 | 6.571 | 75.209 | 58.413 | 1.00 | 55.53 | C |
| ATOM | 22477 | CE1 | PHE | K | 169 | 5.272 | 75.628 | 58.452 | 1.00 | 60.64 | C |
| ATOM | 22479 | CZ | PHE | K | 169 | 4.753 | 76.153 | 59.592 | 1.00 | 46.39 | C |
| ATOM | 22481 | CE2 | PHE | K | 169 | 5.519 | 76.226 | 60.706 | 1.00 | 56.21 | C |
| ATOM | 22483 | CD2 | PHE | K | 169 | 6.820 | 75.797 | 60.670 | 1.00 | 53.02 | C |
| ATOM | 22485 | C | PHE | K | 169 | 11.101 | 75.157 | 59.081 | 1.00 | 47.84 | C |
| ATOM | 22486 | O | PHE | K | 169 | 11.262 | 74.023 | 58.596 | 1.00 | 52.43 | O |
| ATOM | 22487 | N | SER | K | 170 | 12.030 | 75.831 | 59.752 | 1.00 | 38.34 | N |
| ATOM | 22489 | CA | SER | K | 170 | 13.391 | 75.345 | 59.764 | 1.00 | 43.58 | C |
| ATOM | 22491 | CB | SER | K | 170 | 14.277 | 76.117 | 58.769 | 1.00 | 43.17 | C |
| ATOM | 22494 | OG | SER | K | 170 | 14.263 | 77.515 | 59.000 | 1.00 | 61.25 | O |
| ATOM | 22496 | C | SER | K | 170 | 13.913 | 75.400 | 61.198 | 1.00 | 50.19 | C |
| ATOM | 22497 | O | SER | K | 170 | 13.522 | 76.266 | 62.000 | 1.00 | 45.95 | O |
| ATOM | 22498 | N | ALA | K | 171 | 14.764 | 74.424 | 61.501 | 1.00 | 43.71 | N |
| ATOM | 22500 | CA | ALA | K | 171 | 15.239 | 74.180 | 62.834 | 1.00 | 40.76 | C |
| ATOM | 22502 | CB | ALA | K | 171 | 14.455 | 73.048 | 63.423 | 1.00 | 42.57 | C |
| ATOM | 22506 | C | ALA | K | 171 | 16.706 | 73.794 | 62.751 | 1.00 | 46.64 | C |
| ATOM | 22507 | O | ALA | K | 171 | 17.167 | 73.187 | 61.791 | 1.00 | 40.69 | O |
| ATOM | 22508 | N | SER | K | 172 | 17.434 | 74.038 | 63.824 | 1.00 | 52.20 | N |
| ATOM | 22510 | CA | SER | K | 172 | 18.868 | 73.840 | 63.743 | 1.00 | 52.98 | C |
| ATOM | 22512 | CB | SER | K | 172 | 19.514 | 75.150 | 63.327 | 1.00 | 48.89 | C |
| ATOM | 22515 | OG | SER | K | 172 | 20.832 | 74.852 | 62.952 | 1.00 | 62.49 | O |
| ATOM | 22517 | C | SER | K | 172 | 19.438 | 73.371 | 5.072 | 1.00 | 46.13 | C |
| ATOM | 22518 | O | SER | K | 172 | 19.059 | 73.859 | 66.134 | 1.00 | 46.46 | O |
| ATOM | 22519 | N | GLY | K | 173 | 20.362 | 72.430 | 66.021 | 1.00 | 42.79 | N |
| ATOM | 22521 | CA | GLY | K | 173 | 20.798 | 71.779 | 66.242 | 1.00 | 46.15 | C |
| ATOM | 22524 | C | GLY | K | 173 | 22.085 | 70.983 | 66.131 | 1.00 | 54.08 | C |
| ATOM | 22525 | O | GLY | K | 173 | 22.668 | 70.824 | 65.059 | 1.00 | 55.34 | O |
| ATOM | 22526 | N | GLU | K | 174 | 22.530 | 70.451 | 67.261 | 1.00 | 63.34 | N |
| ATOM | 22528 | CA | GLU | K | 174 | 23.833 | 69.813 | 67.334 | 1.00 | 66.18 | C |
| ATOM | 22530 | CB | GLU | K | 174 | 24.046 | 69.244 | 68.740 | 1.00 | 75.54 | C |
| ATOM | 22533 | CG | GLU | K | 174 | 25.494 | 68.973 | 69.146 | 1.00 | 94.41 | C |
| ATOM | 22536 | CD | GLU | K | 174 | 26.347 | 70.230 | 69.201 | 1.00 | 112.42 | C |
| ATOM | 22537 | OE1 | GLU | K | 174 | 27.080 | 70.504 | 68.217 | 1.00 | 107.68 | O |
| ATOM | 22538 | OE2 | GLU | K | 174 | 26.274 | 70.938 | 70.231 | 1.00 | 114.97 | O |
| ATOM | 22539 | C | GLU | K | 174 | 24.020 | 68.730 | 66.276 | 1.00 | 60.29 | C |
| ATOM | 22540 | O | GLU | K | 174 | 25.104 | 68.630 | 65.726 | 1.00 | 62.08 | O |
| ATOM | 22541 | N | LEU | K | 175 | 22.979 | 67.959 | 65.955 | 1.00 | 61.44 | N |
| ATOM | 22543 | CA | LEU | K | 175 | 23.061 | 66.851 | 64.972 | 1.00 | 58.23 | C |
| ATOM | 22545 | CB | LEU | K | 175 | 22.020 | 65.778 | 65.290 | 1.00 | 55.29 | C |
| ATOM | 22548 | CG | LEU | K | 175 | 20.577 | 65.887 | 64.787 | 1.00 | 56.77 | C |
| ATOM | 22550 | CD1 | LEU | K | 175 | 20.485 | 65.366 | 63.408 | 1.00 | 50.87 | C |
| ATOM | 22554 | CD2 | LEU | K | 175 | 19.560 | 65.119 | 65.664 | 1.00 | 70.88 | C |
| ATOM | 22558 | C | LEU | K | 175 | 23.016 | 67.145 | 63.451 | 1.00 | 53.54 | C |
| ATOM | 22559 | O | LEU | K | 175 | 23.247 | 66.268 | 62.608 | 1.00 | 57.97 | O |
| ATOM | 22560 | N | GLY | K | 176 | 22.776 | 68.397 | 63.101 | 1.00 | 51.21 | N |
| ATOM | 22562 | CA | GLY | K | 176 | 22.321 | 68.722 | 61.774 | 1.00 | 48.81 | C |
| ATOM | 22565 | C | GLY | K | 176 | 21.236 | 69.767 | 61.809 | 1.00 | 44.67 | C |
| ATOM | 22566 | O | GLY | K | 176 | 20.850 | 70.258 | 62.864 | 1.00 | 51.34 | O |
| ATOM | 22567 | N | ASN | K | 177 | 20.739 | 70.113 | 60.634 | 1.00 | 45.74 | N |
| ATOM | 22569 | CA | ASN | K | 177 | 19.628 | 71.051 | 60.554 | 1.00 | 55.02 | C |
| ATOM | 22571 | CB | ASN | K | 177 | 20.163 | 72.428 | 60.150 | 1.00 | 56.95 | C |
| ATOM | 22574 | CG | ASN | K | 177 | 20.887 | 72.397 | 58.817 | 1.00 | 51.48 | C |
| ATOM | 22575 | OD1 | ASN | K | 177 | 20.272 | 72.225 | 57.773 | 1.00 | 48.29 | O |
| ATOM | 22576 | ND2 | ASN | K | 177 | 22.199 | 72.549 | 58.852 | 1.00 | 54.97 | N |
| ATOM | 22579 | C | ASN | K | 177 | 18.519 | 70.607 | 59.593 | 1.00 | 49.82 | C |
| ATOM | 22580 | O | ASN | K | 177 | 18.677 | 69.663 | 58.818 | 1.00 | 46.54 | O |
| ATOM | 22581 | N | GLY | K | 178 | 17.418 | 71.341 | 59.623 | 1.00 | 46.07 | N |
| ATOM | 22583 | CA | GLY | K | 178 | 16.159 | 70.823 | 59.143 | 1.00 | 48.94 | C |
| ATOM | 22586 | C | GLY | K | 178 | 15.287 | 71.920 | 58.596 | 1.00 | 44.78 | C |
| ATOM | 22587 | O | GLY | K | 178 | 15.147 | 72.986 | 59.168 | 1.00 | 49.42 | O |
| ATOM | 22588 | N | ASN | K | 179 | 14.589 | 71.615 | 57.524 | 1.00 | 49.34 | N |
| ATOM | 22590 | CA | ASN | K | 179 | 14.014 | 72.655 | 56.705 | 1.00 | 49.41 | C |
| ATOM | 22592 | CB | ASN | K | 179 | 15.109 | 73.027 | 55.718 | 1.00 | 59.56 | C |
| ATOM | 22595 | CG | ASN | K | 179 | 15.014 | 74.425 | 55.285 | 1.00 | 66.91 | C |
| ATOM | 22596 | OD1 | ASN | K | 179 | 15.352 | 75.321 | 56.052 | 1.00 | 82.25 | O |
| ATOM | 22597 | ND2 | ASN | K | 179 | 14.499 | 74.641 | 54.071 | 1.00 | 85.56 | N |
| ATOM | 22600 | C | ASN | K | 179 | 12.821 | 72.009 | 56.011 | 1.00 | 47.78 | C |
| ATOM | 22601 | O | ASN | K | 179 | 13.017 | 71.064 | 55.220 | 1.00 | 40.94 | O |
| ATOM | 22602 | N | ILE | K | 180 | 11.612 | 72.402 | 56.421 | 1.00 | 42.68 | N |
| ATOM | 22604 | CA | ILE | K | 180 | 10.353 | 71.880 | 55.847 | 1.00 | 40.96 | C |
| ATOM | 22606 | CB | ILE | K | 180 | 9.458 | 71.278 | 56.961 | 1.00 | 42.52 | C |
| ATOM | 22608 | CG1 | ILE | K | 180 | 10.194 | 70.139 | 57.648 | 1.00 | 45.97 | C |
| ATOM | 22611 | CD1 | ILE | K | 180 | 9.666 | 69.853 | 58.998 | 1.00 | 42.85 | C |
| ATOM | 22615 | CG2 | ILE | K | 180 | 8.111 | 70.780 | 56.422 | 1.00 | 29.93 | C |
| ATOM | 22619 | C | ILE | K | 180 | 9.559 | 72.993 | 55.157 | 1.00 | 38.50 | C |
| ATOM | 22620 | O | ILE | K | 180 | 9.285 | 74.022 | 55.766 | 1.00 | 36.22 | O |
| ATOM | 22621 | N | LYS | K | 181 | 9.179 | 72.783 | 53.903 | 1.00 | 41.39 | N |
| ATOM | 22623 | CA | LYS | K | 181 | 8.563 | 73.832 | 53.082 | 1.00 | 46.23 | C |
| ATOM | 22625 | CB | LYS | K | 181 | 9.437 | 74.065 | 51.844 | 1.00 | 50.96 | C |
| ATOM | 22628 | CG | LYS | K | 181 | 9.992 | 75.473 | 51.591 | 1.00 | 73.04 | C |
| ATOM | 22631 | CD | LYS | K | 181 | 11.408 | 75.443 | 50.911 | 1.00 | 89.08 | C |
| ATOM | 22634 | CE | LYS | K | 181 | 11.374 | 75.496 | 49.357 | 1.00 | 100.26 | C |
| ATOM | 22637 | NZ | LYS | K | 181 | 11.701 | 76.810 | 48.696 | 1.00 | 95.91 | N |
| ATOM | 22641 | C | LYS | K | 181 | 7.195 | 73.329 | 52.642 | 1.00 | 39.01 | C |
| ATOM | 22642 | O | LYS | K | 181 | 7.112 | 72.267 | 52.028 | 1.00 | 46.57 | O |
| ATOM | 22643 | N | LEU | K | 182 | 6.131 | 74.049 | 52.996 | 1.00 | 45.42 | N |
| ATOM | 22645 | CA | LEU | K | 182 | 4.745 | 73.722 | 52.590 | 1.00 | 45.17 | C |
| ATOM | 22647 | CB | LEU | K | 182 | 3.774 | 73.849 | 53.756 | 1.00 | 39.46 | C |
| ATOM | 22650 | CG | LEU | K | 182 | 3.860 | 72.818 | 54.865 | 1.00 | 45.17 | C |
| ATOM | 22652 | CD1 | LEU | K | 182 | 2.684 | 73.110 | 55.775 | 1.00 | 40.74 | C |
| ATOM | 22656 | CD2 | LEU | K | 182 | 3.807 | 71.405 | 54.283 | 1.00 | 60.19 | C |
| ATOM | 22660 | C | LEU | K | 182 | 4.223 | 74.744 | 51.621 | 1.00 | 43.41 | C |
| ATOM | 22661 | O | LEU | K | 182 | 4.271 | 75.932 | 51.935 | 1.00 | 52.79 | O |
| ATOM | 22662 | N | SER | K | 183 | 3.638 | 74.304 | 50.516 | 1.00 | 45.72 | N |
| ATOM | 22664 | CA | SER | K | 183 | 3.115 | 75.252 | 49.536 | 1.00 | 48.96 | C |
| ATOM | 22666 | CB | SER | K | 183 | 3.356 | 74.701 | 48.147 | 1.00 | 49.23 | C |
| ATOM | 22669 | OG | SER | K | 183 | 4.662 | 74.187 | 48.109 | 1.00 | 66.31 | O |
| ATOM | 22671 | C | SER | K | 183 | 1.633 | 75.558 | 49.730 | 1.00 | 51.93 | C |
| ATOM | 22672 | O | SER | K | 183 | 0.965 | 74.881 | 50.510 | 1.00 | 55.43 | O |
| ATOM | 22673 | N | GLN | K | 184 | 1.130 | 76.588 | 49.049 | 1.00 | 56.46 | N |
| ATOM | 22675 | CA | GLN | K | 184 | −0.297 | 76.889 | 49.032 | 1.00 | 57.97 | C |
| ATOM | 22677 | CB | GLN | K | 184 | −0.507 | 78.290 | 48.506 | 1.00 | 56.24 | C |
| ATOM | 22680 | CG | GLN | K | 184 | −0.662 | 79.314 | 49.596 | 1.00 | 74.33 | C |
| ATOM | 22683 | CD | GLN | K | 184 | −1.469 | 80.531 | 49.155 | 1.00 | 83.96 | C |
| ATOM | 22684 | OE1 | GLN | K | 184 | −0.932 | 81.644 | 49.067 | 1.00 | 88.85 | O |
| ATOM | 22685 | NE2 | GLN | K | 184 | −2.761 | 80.326 | 48.903 | 1.00 | 64.09 | N |
| ATOM | 22688 | C | GLN | K | 184 | −1.002 | 75.975 | 48.064 | 1.00 | 64.87 | C |
| ATOM | 22689 | O | GLN | K | 184 | −0.589 | 75.920 | 46.928 | 1.00 | 72.66 | O |
| ATOM | 22690 | N | THR | K | 185 | −2.092 | 75.332 | 48.462 | 1.00 | 68.71 | N |
| ATOM | 22692 | CA | THR | K | 185 | −2.711 | 74.314 | 47.633 | 1.00 | 77.04 | C |
| ATOM | 22694 | CB | THR | K | 185 | −3.278 | 73.259 | 48.543 | 1.00 | 81.40 | C |
| ATOM | 22696 | OG1 | THR | K | 185 | −3.733 | 73.912 | 49.736 | 1.00 | 74.81 | O |
| ATOM | 22698 | CG2 | THR | K | 185 | −2.176 | 72.266 | 48.968 | 1.00 | 84.93 | C |
| ATOM | 22702 | C | THR | K | 185 | −3.838 | 74.944 | 46.852 | 1.00 | 82.25 | C |
| ATOM | 22703 | O | THR | K | 185 | −3.917 | 76.155 | 46.856 | 1.00 | 83.63 | O |
| ATOM | 22704 | N | SER | K | 186 | −4.706 | 74.140 | 46.222 | 1.00 | 93.08 | N |
| ATOM | 22706 | CA | SER | K | 186 | −5.964 | 74.621 | 45.616 | 1.00 | 96.52 | C |
| ATOM | 22708 | CB | SER | K | 186 | −5.663 | 75.613 | 44.479 | 1.00 | 96.78 | C |
| ATOM | 22711 | OG | SER | K | 186 | −4.632 | 75.120 | 43.631 | 1.00 | 90.11 | O |
| ATOM | 22713 | C | SER | K | 186 | −6.884 | 73.502 | 45.082 | 1.00 | 98.63 | C |
| ATOM | 22714 | O | SER | K | 186 | −7.069 | 73.355 | 43.870 | 1.00 | 99.41 | O |
| ATOM | 22715 | N | ASN | K | 187 | −7.489 | 72.729 | 45.981 | 1.00 | 98.97 | N |
| ATOM | 22717 | CA | ASN | K | 187 | −8.518 | 71.747 | 45.608 | 1.00 | 101.80 | C |
| ATOM | 22719 | CB | ASN | K | 187 | −8.318 | 71.277 | 44.145 | 1.00 | 99.35 | C |
| ATOM | 22722 | CG | ASN | K | 187 | −9.594 | 70.688 | 43.520 | 1.00 | 103.81 | C |
| ATOM | 22723 | OD1 | ASN | K | 187 | −9.722 | 70.520 | 42.279 | 1.00 | 71.87 | O |
| ATOM | 22724 | ND2 | ASN | K | 187 | −10.545 | 70.349 | 44.396 | 1.00 | 78.95 | N |
| ATOM | 22727 | C | ASN | K | 187 | −8.575 | 70.566 | 46.618 | 1.00 | 105.22 | C |
| ATOM | 22728 | O | ASN | K | 187 | −7.566 | 69.892 | 46.877 | 1.00 | 97.28 | O |
| ATOM | 22729 | N | VAL | K | 188 | −9.734 | 70.402 | 47.259 | 1.00 | 110.13 | N |
| ATOM | 22731 | CA | VAL | K | 188 | −10.281 | 69.106 | 47.707 | 1.00 | 118.69 | C |
| ATOM | 22733 | CB | VAL | K | 188 | −9.248 | 67.955 | 47.696 | 1.00 | 119.20 | C |
| ATOM | 22735 | CG1 | VAL | K | 188 | −8.127 | 68.232 | 48.696 | 1.00 | 121.14 | C |
| ATOM | 22739 | CG2 | VAL | K | 188 | −9.944 | 66.615 | 47.989 | 1.00 | 111.14 | C |
| ATOM | 22743 | C | VAL | K | 188 | −10.910 | 69.195 | 49.109 | 1.00 | 126.25 | C |
| ATOM | 22744 | O | VAL | K | 188 | −11.154 | 68.183 | 49.784 | 1.00 | 125.40 | O |
| ATOM | 22745 | N | ASP | K | 189 | −11.191 | 70.436 | 49.501 | 1.00 | 132.13 | N |
| ATOM | 22747 | CA | ASP | K | 189 | −11.709 | 70.826 | 50.813 | 1.00 | 132.88 | C |
| ATOM | 22749 | CB | ASP | K | 189 | −12.852 | 69.932 | 51.311 | 1.00 | 134.19 | C |
| ATOM | 22752 | CG | ASP | K | 189 | −14.077 | 70.745 | 51.741 | 1.00 | 135.86 | C |
| ATOM | 22753 | OD1 | ASP | K | 189 | −15.032 | 70.166 | 52.306 | 1.00 | 123.15 | O |
| ATOM | 22754 | OD2 | ASP | K | 189 | −14.168 | 71.982 | 51.564 | 1.00 | 131.98 | O |
| ATOM | 22755 | C | ASP | K | 189 | −10.627 | 71.004 | 51.865 | 1.00 | 132.01 | C |
| ATOM | 22756 | O | ASP | K | 189 | −9.577 | 70.360 | 51.809 | 1.00 | 129.81 | O |
| ATOM | 22757 | N | LYS | K | 190 | −10.917 | 71.889 | 52.818 | 1.00 | 133.28 | N |
| ATOM | 22759 | CA | LYS | K | 190 | −9.994 | 72.942 | 53.246 | 1.00 | 132.59 | C |
| ATOM | 22761 | CB | LYS | K | 190 | −10.748 | 74.255 | 53.546 | 1.00 | 132.18 | C |
| ATOM | 22764 | CG | LYS | K | 190 | −12.288 | 74.169 | 53.600 | 1.00 | 126.69 | C |
| ATOM | 22767 | CD | LYS | K | 190 | −12.786 | 73.573 | 54.930 | 1.00 | 122.94 | C |
| ATOM | 22770 | CE | LYS | K | 190 | −14.175 | 74.047 | 55.301 | 1.00 | 109.83 | C |
| ATOM | 22773 | NZ | LYS | K | 190 | −14.478 | 75.257 | 54.505 | 1.00 | 102.19 | N |
| ATOM | 22777 | C | LYS | K | 190 | −9.144 | 72.482 | 54.448 | 1.00 | 132.51 | C |
| ATOM | 22778 | O | LYS | K | 190 | −7.912 | 72.534 | 54.408 | 1.00 | 135.07 | O |
| ATOM | 22779 | N | GLU | K | 191 | −9.794 | 72.004 | 55.506 | 1.00 | 131.61 | N |
| ATOM | 22781 | CA | GLU | K | 191 | −9.438 | 70.722 | 56.104 | 1.00 | 129.12 | C |
| ATOM | 22783 | CB | GLU | K | 191 | −10.721 | 69.945 | 56.416 | 1.00 | 127.14 | C |
| ATOM | 22786 | CG | GLU | K | 191 | −10.615 | 68.989 | 57.587 | 1.00 | 122.98 | C |
| ATOM | 22789 | CD | GLU | K | 191 | −10.445 | 67.555 | 57.139 | 1.00 | 122.67 | C |
| ATOM | 22790 | OE1 | GLU | K | 191 | −9.452 | 66.896 | 57.534 | 1.00 | 123.38 | O |
| ATOM | 22791 | OE2 | GLU | K | 191 | −11.312 | 67.100 | 56.370 | 1.00 | 113.32 | O |
| ATOM | 22792 | C | GLU | K | 191 | −8.580 | 69.944 | 55.104 | 1.00 | 131.11 | C |
| ATOM | 22793 | O | GLU | K | 191 | −9.044 | 69.633 | 54.002 | 1.00 | 134.39 | O |
| ATOM | 22794 | N | GLU | K | 192 | −7.328 | 69.660 | 55.466 | 1.00 | 129.16 | N |
| ATOM | 22796 | CA | GLU | K | 192 | −6.461 | 68.765 | 54.681 | 1.00 | 126.40 | C |
| ATOM | 22798 | CB | GLU | K | 192 | −7.250 | 67.592 | 54.070 | 1.00 | 130.08 | C |
| ATOM | 22801 | CG | GLU | K | 192 | −7.518 | 67.696 | 52.567 | 1.00 | 132.71 | C |
| ATOM | 22804 | CD | GLU | K | 192 | −6.455 | 67.033 | 51.697 | 1.00 | 126.47 | C |
| ATOM | 22805 | OE1 | GLU | K | 192 | −6.718 | 66.826 | 50.491 | 1.00 | 118.53 | O |
| ATOM | 22806 | OE2 | GLU | K | 192 | −5.349 | 66.734 | 52.198 | 1.00 | 122.52 | O |
| ATOM | 22807 | C | GLU | K | 192 | −5.653 | 69.462 | 53.578 | 1.00 | 119.47 | C |
| ATOM | 22808 | O | GLU | K | 192 | −6.155 | 70.359 | 52.888 | 1.00 | 108.43 | O |
| ATOM | 22809 | N | GLU | K | 193 | −4.416 | 68.990 | 53.394 | 1.00 | 115.12 | N |
| ATOM | 22811 | CA | GLU | K | 193 | −3.279 | 69.859 | 53.078 | 1.00 | 111.16 | C |
| ATOM | 22813 | CB | GLU | K | 193 | −2.180 | 69.117 | 52.267 | 1.00 | 112.21 | C |
| ATOM | 22816 | CG | GLU | K | 193 | −0.739 | 69.634 | 52.405 | 1.00 | 114.20 | C |
| ATOM | 22819 | CD | GLU | K | 193 | 0.220 | 68.666 | 53.120 | 1.00 | 120.01 | C |
| ATOM | 22820 | OE1 | GLU | K | 193 | 0.944 | 67.878 | 52.457 | 1.00 | 101.32 | O |
| ATOM | 22821 | OE2 | GLU | K | 193 | 0.289 | 68.693 | 54.372 | 1.00 | 110.45 | O |
| ATOM | 22822 | C | GLU | K | 193 | −3.870 | 71.011 | 52.277 | 1.00 | 102.89 | C |
| ATOM | 22823 | O | GLU | K | 193 | −3.471 | 71.200 | 51.126 | 1.00 | 114.50 | O |
| ATOM | 22824 | N | ALA | K | 194 | −4.833 | 71.739 | 52.851 | 1.00 | 82.00 | N |
| ATOM | 22826 | CA | ALA | K | 194 | −5.204 | 73.035 | 52.305 | 1.00 | 67.76 | C |
| ATOM | 22828 | CB | ALA | K | 194 | −6.671 | 73.240 | 52.344 | 1.00 | 60.92 | C |
| ATOM | 22832 | C | ALA | K | 194 | −4.472 | 74.114 | 53.074 | 1.00 | 65.27 | C |
| ATOM | 22833 | O | ALA | K | 194 | −4.980 | 74.698 | 54.030 | 1.00 | 73.03 | O |
| ATOM | 22834 | N | VAL | K | 195 | −3.218 | 74.303 | 52.693 | 1.00 | 58.24 | N |
| ATOM | 22836 | CA | VAL | K | 195 | −2.440 | 75.449 | 53.130 | 1.00 | 56.47 | C |
| ATOM | 22838 | CB | VAL | K | 195 | −0.915 | 75.200 | 52.936 | 1.00 | 52.55 | C |
| ATOM | 22840 | CG1 | VAL | K | 195 | −0.128 | 76.377 | 53.449 | 1.00 | 47.95 | C |
| ATOM | 22844 | CG2 | VAL | K | 195 | −0.470 | 73.933 | 53.623 | 1.00 | 54.93 | C |
| ATOM | 22848 | C | VAL | K | 195 | −2.815 | 76.672 | 52.302 | 1.00 | 52.86 | C |
| ATOM | 22849 | O | VAL | K | 195 | −2.812 | 76.608 | 51.076 | 1.00 | 58.59 | O |
| ATOM | 22850 | N | THR | K | 196 | −3.082 | 77.784 | 52.976 | 1.00 | 46.20 | N |
| ATOM | 22852 | CA | THR | K | 196 | −3.494 | 79.020 | 52.322 | 1.00 | 50.89 | C |
| ATOM | 22854 | CB | THR | K | 196 | −5.068 | 79.090 | 52.134 | 1.00 | 51.31 | C |
| ATOM | 22856 | OG1 | THR | K | 196 | −5.703 | 79.683 | 53.271 | 1.00 | 50.45 | O |
| ATOM | 22858 | CG2 | THR | K | 196 | −5.746 | 77.701 | 52.070 | 1.00 | 54.44 | C |
| ATOM | 22862 | C | THR | K | 196 | −2.918 | 80.225 | 53.090 | 1.00 | 50.51 | C |
| ATOM | 22863 | O | THR | K | 196 | −2.920 | 80.247 | 54.318 | 1.00 | 54.99 | O |
| ATOM | 22864 | N | ILE | K | 197 | −2.329 | 81.181 | 52.380 | 1.00 | 51.19 | N |
| ATOM | 22866 | CA | ILE | K | 197 | −1.529 | 82.227 | 53.020 | 1.00 | 51.42 | C |
| ATOM | 22868 | CB | ILE | K | 197 | −0.102 | 82.257 | 52.431 | 1.00 | 46.89 | C |
| ATOM | 22870 | CG1 | ILE | K | 197 | 0.462 | 80.847 | 52.294 | 1.00 | 54.10 | C |
| ATOM | 22873 | CD1 | ILE | K | 197 | 1.502 | 80.483 | 53.312 | 1.00 | 56.89 | C |
| ATOM | 22877 | CG2 | ILE | K | 197 | 0.858 | 83.054 | 53.294 | 1.00 | 57.17 | C |
| ATOM | 22881 | C | ILE | K | 197 | −2.227 | 83.567 | 52.797 | 1.00 | 57.89 | C |
| ATOM | 22882 | O | ILE | K | 197 | −3.132 | 83.657 | 51.970 | 1.00 | 72.70 | O |
| ATOM | 22883 | N | GLU | K | 198 | −1.855 | 84.581 | 53.576 | 1.00 | 64.92 | N |
| ATOM | 22885 | CA | GLU | K | 198 | −2.437 | 85.920 | 53.504 | 1.00 | 68.49 | C |
| ATOM | 22887 | CB | GLU | K | 198 | −3.522 | 86.131 | 54.556 | 1.00 | 65.94 | C |
| ATOM | 22890 | CG | GLU | K | 198 | −4.825 | 85.421 | 54.226 | 1.00 | 85.91 | C |
| ATOM | 22893 | CD | GLU | K | 198 | −5.559 | 86.093 | 53.080 | 1.00 | 106.49 | C |
| ATOM | 22894 | OE1 | GLU | K | 198 | −6.234 | 87.117 | 53.345 | 1.00 | 120.31 | O |
| ATOM | 22895 | OE2 | GLU | K | 198 | −5.448 | 85.615 | 51.924 | 1.00 | 103.13 | O |
| ATOM | 22896 | C | GLU | K | 198 | −1.288 | 86.863 | 53.750 | 1.00 | 71.77 | C |
| ATOM | 22897 | O | GLU | K | 198 | −1.049 | 87.315 | 54.864 | 1.00 | 77.04 | O |
| ATOM | 22898 | N | MET | K | 199 | −0.520 | 87.078 | 52.694 | 1.00 | 82.67 | N |
| ATOM | 22900 | CA | MET | K | 199 | 0.820 | 87.640 | 52.807 | 1.00 | 85.10 | C |
| ATOM | 22902 | CB | MET | K | 199 | 1.718 | 87.024 | 51.735 | 1.00 | 82.12 | C |
| ATOM | 22905 | CG | MET | K | 199 | 3.121 | 86.750 | 52.205 | 1.00 | 88.03 | C |
| ATOM | 22908 | SD | MET | K | 199 | 4.173 | 88.197 | 52.093 | 1.00 | 112.95 | S |
| ATOM | 22909 | CE | MET | K | 199 | 5.659 | 87.497 | 51.394 | 1.00 | 115.41 | C |
| ATOM | 22913 | C | MET | K | 199 | 0.691 | 89.124 | 52.527 | 1.00 | 87.30 | C |
| ATOM | 22914 | O | MET | K | 199 | 0.714 | 89.518 | 51.360 | 1.00 | 91.87 | O |
| ATOM | 22915 | N | ASN | K | 200 | 0.463 | 89.923 | 53.567 | 1.00 | 84.98 | N |
| ATOM | 22917 | CA | ASN | K | 200 | 0.611 | 91.370 | 53.455 | 1.00 | 83.30 | C |
| ATOM | 22919 | CB | ASN | K | 200 | −0.048 | 92.069 | 54.647 | 1.00 | 83.28 | C |
| ATOM | 22922 | CG | ASN | K | 200 | −1.474 | 92.489 | 54.353 | 1.00 | 92.30 | C |
| ATOM | 22923 | OD1 | ASN | K | 200 | −2.060 | 92.069 | 53.354 | 1.00 | 91.20 | O |
| ATOM | 22924 | ND2 | ASN | K | 200 | −2.030 | 93.345 | 55.203 | 1.00 | 90.71 | N |
| ATOM | 22927 | C | ASN | K | 200 | 2.079 | 91.774 | 53.343 | 1.00 | 82.33 | C |
| ATOM | 22928 | O | ASN | K | 200 | 2.404 | 92.740 | 52.664 | 1.00 | 84.01 | O |
| ATOM | 22929 | N | GLU | K | 201 | 2.959 | 90.998 | 53.973 | 1.00 | 83.80 | N |
| ATOM | 22931 | CA | GLU | K | 201 | 4.385 | 91.308 | 54.080 | 1.00 | 83.24 | C |
| ATOM | 22933 | CB | GLU | K | 201 | 4.578 | 92.517 | 55.012 | 1.00 | 85.87 | C |
| ATOM | 22936 | CG | GLU | K | 201 | 5.932 | 93.224 | 54.921 | 1.00 | 101.42 | C |
| ATOM | 22939 | CD | GLU | K | 201 | 6.164 | 94.216 | 56.060 | 1.00 | 120.07 | C |
| ATOM | 22940 | OE1 | GLU | K | 201 | 5.175 | 94.792 | 56.570 | 1.00 | 129.94 | O |
| ATOM | 22941 | OE2 | GLU | K | 201 | 7.335 | 94.429 | 56.457 | 1.00 | 118.46 | O |
| ATOM | 22942 | C | GLU | K | 201 | 5.137 | 90.062 | 54.602 | 1.00 | 79.51 | C |
| ATOM | 22943 | O | GLU | K | 201 | 4.710 | 89.447 | 55.581 | 1.00 | 71.50 | O |
| ATOM | 22944 | N | PRO | K | 202 | 6.263 | 89.686 | 53.995 | 1.00 | 75.77 | N |
| ATOM | 22945 | CA | PRO | K | 202 | 7.003 | 88.531 | 54.498 | 1.00 | 73.44 | C |
| ATOM | 22947 | CB | PRO | K | 202 | 8.261 | 88.458 | 53.620 | 1.00 | 71.24 | C |
| ATOM | 22950 | CG | PRO | K | 202 | 8.093 | 89.441 | 52.546 | 1.00 | 77.45 | C |
| ATOM | 22953 | CD | PRO | K | 202 | 7.014 | 90.400 | 52.950 | 1.00 | 81.31 | C |
| ATOM | 22956 | C | PRO | K | 202 | 7.397 | 88.797 | 55.948 | 1.00 | 72.52 | C |
| ATOM | 22957 | O | PRO | K | 202 | 7.566 | 89.954 | 56.364 | 1.00 | 70.45 | O |
| ATOM | 22958 | N | VAL | K | 203 | 7.507 | 87.699 | 56.690 | 1.00 | 68.42 | N |
| ATOM | 22960 | CA | VAL | K | 203 | 7.861 | 87.671 | 58.098 | 1.00 | 58.56 | C |
| ATOM | 22962 | CB | VAL | K | 203 | 6.626 | 87.390 | 58.940 | 1.00 | 56.70 | C |
| ATOM | 22964 | CG1 | VAL | K | 203 | 7.051 | 87.008 | 60.330 | 1.00 | 68.94 | C |
| ATOM | 22968 | CG2 | VAL | K | 203 | 5.645 | 88.571 | 58.959 | 1.00 | 50.35 | C |
| ATOM | 22972 | C | VAL | K | 203 | 8.704 | 86.425 | 58.230 | 1.00 | 57.04 | C |
| ATOM | 22973 | O | VAL | K | 203 | 8.278 | 85.369 | 57.755 | 1.00 | 66.44 | O |
| ATOM | 22974 | N | GLN | K | 204 | 9.875 | 86.528 | 58.849 | 1.00 | 52.93 | N |
| ATOM | 22976 | CA | GLN | K | 204 | 10.604 | 85.354 | 59.332 | 1.00 | 49.38 | C |
| ATOM | 22978 | CB | GLN | K | 204 | 11.828 | 85.157 | 58.462 | 1.00 | 52.07 | C |
| ATOM | 22981 | CG | GLN | K | 204 | 13.148 | 84.914 | 59.187 | 1.00 | 68.25 | C |
| ATOM | 22984 | CD | GLN | K | 204 | 14.289 | 84.630 | 58.201 | 1.00 | 76.26 | C |
| ATOM | 22985 | OE1 | GLN | K | 204 | 14.190 | 83.724 | 57.395 | 1.00 | 73.03 | O |
| ATOM | 22986 | NE2 | GLN | K | 204 | 15.333 | 85.442 | 58.224 | 1.00 | 96.66 | N |
| ATOM | 22989 | C | GLN | K | 204 | 10.962 | 85.463 | 60.823 | 1.00 | 52.49 | C |
| ATOM | 22990 | O | GLN | K | 204 | 11.784 | 86.282 | 61.234 | 1.00 | 58.96 | O |
| ATOM | 22991 | N | LEU | K | 205 | 10.290 | 84.661 | 61.644 | 1.00 | 57.20 | N |
| ATOM | 22993 | CA | LEU | K | 205 | 10.267 | 84.834 | 63.093 | 1.00 | 57.56 | C |
| ATOM | 22995 | CB | LEU | K | 205 | 8.842 | 85.186 | 63.492 | 1.00 | 58.10 | C |
| ATOM | 22998 | CG | LEU | K | 205 | 8.547 | 86.635 | 63.148 | 1.00 | 71.68 | C |
| ATOM | 23000 | CD1 | LEU | K | 205 | 7.168 | 87.004 | 63.649 | 1.00 | 83.41 | C |
| ATOM | 23004 | CD2 | LEU | K | 205 | 9.613 | 87.539 | 63.759 | 1.00 | 90.45 | C |
| ATOM | 23008 | C | LEU | K | 205 | 10.662 | 83.522 | 63.760 | 1.00 | 50.79 | C |
| ATOM | 23009 | O | LEU | K | 205 | 10.472 | 82.501 | 63.148 | 1.00 | 59.21 | O |
| ATOM | 23010 | N | THR | K | 206 | 11.145 | 83.532 | 65.002 | 1.00 | 51.02 | N |
| ATOM | 23012 | CA | THR | K | 206 | 11.720 | 82.348 | 65.676 | 1.00 | 41.25 | C |
| ATOM | 23014 | CB | THR | K | 206 | 13.231 | 82.574 | 66.039 | 1.00 | 26.05 | C |
| ATOM | 23016 | OG1 | THR | K | 206 | 13.992 | 82.595 | 64.826 | 1.00 | 45.00 | O |
| ATOM | 23018 | CG2 | THR | K | 206 | 13.862 | 81.394 | 66.745 | 1.00 | 32.37 | C |
| ATOM | 23022 | C | THR | K | 206 | 10.911 | 81.966 | 66.931 | 1.00 | 41.57 | C |
| ATOM | 23023 | O | THR | K | 206 | 10.426 | 82.838 | 67.660 | 1.00 | 36.40 | O |
| ATOM | 23024 | N | PHE | K | 207 | 10.720 | 80.664 | 67.159 | 1.00 | 37.72 | N |
| ATOM | 23026 | CA | PHE | K | 207 | 9.908 | 80.265 | 68.285 | 1.00 | 42.09 | C |
| ATOM | 23028 | CB | PHE | K | 207 | 8.427 | 80.148 | 67.925 | 1.00 | 38.31 | C |
| ATOM | 23031 | CG | PHE | K | 207 | 7.827 | 81.424 | 67.441 | 1.00 | 37.47 | C |
| ATOM | 23032 | CD1 | PHE | K | 207 | 7.427 | 82.412 | 68.331 | 1.00 | 45.09 | C |
| ATOM | 23034 | CE1 | PHE | K | 207 | 6.885 | 83.606 | 67.852 | 1.00 | 49.83 | C |
| ATOM | 23036 | CZ | PHE | K | 207 | 6.753 | 83.806 | 66.467 | 1.00 | 42.76 | C |
| ATOM | 23038 | CE2 | PHE | K | 207 | 7.152 | 82.825 | 65.597 | 1.00 | 42.91 | C |
| ATOM | 23040 | CD2 | PHE | K | 207 | 7.691 | 81.647 | 66.082 | 1.00 | 43.19 | C |
| ATOM | 23042 | C | PHE | K | 207 | 10.405 | 78.994 | 68.933 | 1.00 | 43.25 | C |
| ATOM | 23043 | O | PHE | K | 207 | 10.924 | 78.108 | 68.277 | 1.00 | 46.06 | O |
| ATOM | 23044 | N | ALA | K | 208 | 10.217 | 78.936 | 70.245 | 1.00 | 43.38 | N |
| ATOM | 23046 | CA | ALA | K | 208 | 10.562 | 77.781 | 71.033 | 1.00 | 44.05 | C |
| ATOM | 23048 | CB | ALA | K | 208 | 10.469 | 78.152 | 72.462 | 1.00 | 46.36 | C |
| ATOM | 23052 | C | ALA | K | 208 | 9.608 | 76.643 | 70.747 | 1.00 | 44.38 | C |
| ATOM | 23053 | O | ALA | K | 208 | 8.403 | 76.728 | 71.038 | 1.00 | 53.08 | O |
| ATOM | 23054 | N | LEU | K | 209 | 10.142 | 75.558 | 70.203 | 1.00 | 40.37 | N |
| ATOM | 23056 | CA | LEU | K | 209 | 9.303 | 74.389 | 69.904 | 1.00 | 43.20 | C |
| ATOM | 23058 | CB | LEU | K | 209 | 10.122 | 73.350 | 69.123 | 1.00 | 38.98 | C |
| ATOM | 23061 | CG | LEU | K | 209 | 10.265 | 73.759 | 67.653 | 1.00 | 47.11 | C |
| ATOM | 23063 | CD1 | LEU | K | 209 | 11.258 | 72.846 | 66.977 | 1.00 | 35.36 | C |
| ATOM | 23067 | CD2 | LEU | K | 209 | 8.906 | 73.781 | 66.902 | 1.00 | 44.14 | C |
| ATOM | 23071 | C | LEU | K | 209 | 8.591 | 73.734 | 71.102 | 1.00 | 38.05 | C |
| ATOM | 23072 | O | LEU | K | 209 | 7.551 | 73.092 | 70.898 | 1.00 | 33.56 | O |
| ATOM | 23073 | N | ARG | K | 210 | 9.186 | 73.898 | 72.296 | 1.00 | 36.00 | N |
| ATOM | 23075 | CA | ARG | K | 210 | 8.736 | 73.373 | 73.585 | 1.00 | 37.10 | C |
| ATOM | 23077 | CB | ARG | K | 210 | 9.586 | 73.943 | 74.759 | 1.00 | 40.58 | C |
| ATOM | 23080 | CG | ARG | K | 210 | 9.027 | 73.553 | 76.165 | 1.00 | 66.77 | C |
| ATOM | 23083 | CD | ARG | K | 210 | 9.836 | 73.882 | 77.461 | 1.00 | 69.42 | C |
| ATOM | 23086 | NE | ARG | K | 210 | 9.926 | 72.740 | 78.395 | 1.00 | 55.88 | N |
| ATOM | 23088 | CZ | ARG | K | 210 | 10.684 | 71.645 | 78.148 | 1.00 | 92.18 | C |
| ATOM | 23089 | NH1 | ARG | K | 210 | 11.375 | 71.532 | 77.006 | 1.00 | 86.27 | N |
| ATOM | 23092 | NH2 | ARG | K | 210 | 10.778 | 70.646 | 79.025 | 1.00 | 91.59 | N |
| ATOM | 23095 | C | ARG | K | 210 | 7.289 | 73.797 | 73.761 | 1.00 | 41.27 | C |
| ATOM | 23096 | O | ARG | K | 210 | 6.402 | 72.976 | 74.009 | 1.00 | 39.90 | O |
| ATOM | 23097 | N | TYR | K | 211 | 7.087 | 75.105 | 73.619 | 1.00 | 37.02 | N |
| ATOM | 23099 | CA | TYR | K | 211 | 5.782 | 75.706 | 73.753 | 1.00 | 40.93 | C |
| ATOM | 23101 | CB | TYR | K | 211 | 5.962 | 77.229 | 73.886 | 1.00 | 48.76 | C |
| ATOM | 23104 | CG | TYR | K | 211 | 6.774 | 77.567 | 75.117 | 1.00 | 45.50 | C |
| ATOM | 23105 | CD1 | TYR | K | 211 | 8.135 | 77.631 | 75.040 | 1.00 | 56.61 | C |
| ATOM | 23107 | CE1 | TYR | K | 211 | 8.895 | 77.859 | 76.146 | 1.00 | 75.53 | C |
| ATOM | 23109 | CZ | TYR | K | 211 | 8.314 | 78.015 | 77.365 | 1.00 | 60.84 | C |
| ATOM | 23110 | OH | TYR | K | 211 | 9.178 | 78.185 | 78.417 | 1.00 | 85.41 | O |
| ATOM | 23112 | CE2 | TYR | K | 211 | 6.953 | 77.965 | 77.491 | 1.00 | 62.23 | C |
| ATOM | 23114 | CD2 | TYR | K | 211 | 6.185 | 77.736 | 76.364 | 1.00 | 58.48 | C |
| ATOM | 23116 | C | TYR | K | 211 | 4.819 | 75.321 | 72.625 | 1.00 | 43.51 | C |
| ATOM | 23117 | O | TYR | K | 211 | 3.609 | 75.144 | 72.842 | 1.00 | 40.28 | O |
| ATOM | 23118 | N | LEU | K | 212 | 5.324 | 75.240 | 71.398 | 1.00 | 43.09 | N |
| ATOM | 23120 | CA | LEU | K | 212 | 4.424 | 74.956 | 70.286 | 1.00 | 37.78 | C |
| ATOM | 23122 | CB | LEU | K | 212 | 5.164 | 75.082 | 68.965 | 1.00 | 41.20 | C |
| ATOM | 23125 | CG | LEU | K | 212 | 5.519 | 76.484 | 68.485 | 1.00 | 35.32 | C |
| ATOM | 23127 | CD1 | LEU | K | 212 | 6.167 | 76.293 | 67.161 | 1.00 | 40.75 | C |
| ATOM | 23131 | CD2 | LEU | K | 212 | 4.289 | 77.329 | 68.325 | 1.00 | 41.79 | C |
| ATOM | 23135 | C | LEU | K | 212 | 3.859 | 73.548 | 70.450 | 1.00 | 42.39 | C |
| ATOM | 23136 | O | LEU | K | 212 | 2.656 | 73.324 | 70.220 | 1.00 | 43.71 | O |
| ATOM | 23137 | N | ASN | K | 213 | 4.703 | 72.617 | 70.911 | 1.00 | 39.90 | N |
| ATOM | 23139 | CA | ASN | K | 213 | 4.279 | 71.220 | 71.089 | 1.00 | 44.77 | C |
| ATOM | 23141 | CB | ASN | K | 213 | 5.516 | 70.327 | 71.241 | 1.00 | 41.68 | C |
| ATOM | 23144 | CG | ASN | K | 213 | 6.210 | 70.044 | 69.913 | 1.00 | 40.21 | C |
| ATOM | 23145 | OD1 | ASN | K | 213 | 5.583 | 69.921 | 68.857 | 1.00 | 41.02 | O |
| ATOM | 23146 | ND2 | ASN | K | 213 | 7.522 | 69.914 | 69.971 | 1.00 | 43.97 | N |
| ATOM | 23149 | C | ASN | K | 213 | 3.262 | 70.961 | 72.239 | 1.00 | 50.33 | C |
| ATOM | 23150 | O | ASN | K | 213 | 2.580 | 69.905 | 72.312 | 1.00 | 45.33 | O |
| ATOM | 23151 | N | PHE | K | 214 | 3.229 | 71.923 | 73.162 | 1.00 | 50.96 | N |
| ATOM | 23153 | CA | PHE | K | 214 | 2.122 | 72.137 | 74.064 | 1.00 | 45.51 | C |
| ATOM | 23155 | CB | PHE | K | 214 | 2.507 | 73.193 | 75.086 | 1.00 | 50.03 | C |
| ATOM | 23158 | CG | PHE | K | 214 | 3.512 | 72.741 | 76.098 | 1.00 | 52.57 | C |
| ATOM | 23159 | CD1 | PHE | K | 214 | 3.398 | 71.522 | 76.720 | 1.00 | 67.68 | C |
| ATOM | 23161 | CE1 | PHE | K | 214 | 4.284 | 71.145 | 77.720 | 1.00 | 48.57 | C |
| ATOM | 23163 | CZ | PHE | K | 214 | 5.304 | 71.965 | 78.068 | 1.00 | 42.39 | C |
| ATOM | 23165 | CE2 | PHE | K | 214 | 5.439 | 73.191 | 77.462 | 1.00 | 50.85 | C |
| ATOM | 23167 | CD2 | PHE | K | 214 | 4.527 | 73.585 | 76.502 | 1.00 | 64.36 | C |
| ATOM | 23169 | C | PHE | K | 214 | 0.894 | 72.637 | 73.319 | 1.00 | 45.08 | C |
| ATOM | 23170 | O | PHE | K | 214 | −0.220 | 72.155 | 73.572 | 1.00 | 45.97 | O |
| ATOM | 23171 | N | PHE | K | 215 | 1.038 | 73.644 | 72.465 | 1.00 | 41.18 | N |
| ATOM | 23173 | CA | PHE | K | 215 | −0.187 | 74.213 | 71.904 | 1.00 | 39.77 | C |
| ATOM | 23175 | CB | PHE | K | 215 | 0.041 | 75.404 | 70.991 | 1.00 | 39.97 | C |
| ATOM | 23178 | CG | PHE | K | 215 | 0.733 | 76.547 | 71.648 | 1.00 | 36.28 | C |
| ATOM | 23179 | CD1 | PHE | K | 215 | 0.801 | 76.622 | 73.011 | 1.00 | 49.13 | C |
| ATOM | 23181 | CE1 | PHE | K | 215 | 1.443 | 77.681 | 73.628 | 1.00 | 42.66 | C |
| ATOM | 23183 | CZ | PHE | K | 215 | 2.055 | 78.635 | 72.900 | 1.00 | 34.63 | C |
| ATOM | 23185 | CE2 | PHE | K | 215 | 1.957 | 78.583 | 71.522 | 1.00 | 57.00 | C |
| ATOM | 23187 | CD2 | PHE | K | 215 | 1.326 | 77.535 | 70.906 | 1.00 | 32.61 | C |
| ATOM | 23189 | C | PHE | K | 215 | −0.865 | 73.131 | 71.107 | 1.00 | 38.52 | C |
| ATOM | 23190 | O | PHE | K | 215 | −2.074 | 73.084 | 71.067 | 1.00 | 40.00 | O |
| ATOM | 23191 | N | THR | K | 216 | −0.115 | 72.268 | 70.440 | 1.00 | 41.48 | N |
| ATOM | 23193 | CA | THR | K | 216 | −0.770 | 71.336 | 69.528 | 1.00 | 39.90 | C |
| ATOM | 23195 | CB | THR | K | 216 | 0.263 | 70.733 | 68.626 | 1.00 | 37.15 | C |
| ATOM | 23197 | OG1 | THR | K | 216 | 1.363 | 70.287 | 69.431 | 1.00 | 41.27 | O |
| ATOM | 23199 | CG2 | THR | K | 216 | 0.804 | 71.807 | 67.722 | 1.00 | 33.96 | C |
| ATOM | 23203 | C | THR | K | 216 | −1.524 | 70.216 | 70.238 | 1.00 | 39.83 | C |
| ATOM | 23204 | O | THR | K | 216 | −2.137 | 69.361 | 69.589 | 1.00 | 40.53 | O |
| ATOM | 23205 | N | LYS | K | 217 | −1.530 | 70.239 | 71.566 | 1.00 | 37.59 | N |
| ATOM | 23207 | CA | LYS | K | 217 | −2.399 | 69.326 | 72.319 | 1.00 | 42.09 | C |
| ATOM | 23209 | CB | LYS | K | 217 | −2.122 | 69.380 | 73.843 | 1.00 | 46.86 | C |
| ATOM | 23212 | CG | LYS | K | 217 | −0.799 | 68.741 | 74.354 | 1.00 | 43.18 | C |
| ATOM | 23215 | CD | LYS | K | 217 | −0.763 | 67.217 | 74.213 | 1.00 | 58.39 | C |
| ATOM | 23218 | CE | LYS | K | 217 | 0.473 | 66.750 | 73.354 | 1.00 | 79.45 | C |
| ATOM | 23221 | NZ | LYS | K | 217 | 0.659 | 67.244 | 71.914 | 1.00 | 55.34 | N |
| ATOM | 23225 | C | LYS | K | 217 | −3.860 | 69.647 | 72.065 | 1.00 | 34.64 | C |
| ATOM | 23226 | O | LYS | K | 217 | −4.736 | 68.916 | 72.472 | 1.00 | 38.23 | O |
| ATOM | 23227 | N | ALA | K | 218 | −4.117 | 70.758 | 71.391 | 1.00 | 43.57 | N |
| ATOM | 23229 | CA | ALA | K | 218 | −5.466 | 71.166 | 71.033 | 1.00 | 44.84 | C |
| ATOM | 23231 | CB | ALA | K | 218 | −5.545 | 72.664 | 71.156 | 1.00 | 50.32 | C |
| ATOM | 23235 | C | ALA | K | 218 | −5.904 | 70.712 | 69.631 | 1.00 | 44.87 | C |
| ATOM | 23236 | O | ALA | K | 218 | −6.969 | 71.100 | 69.158 | 1.00 | 57.42 | O |
| ATOM | 23237 | N | THR | K | 219 | −5.121 | 69.836 | 69.005 | 1.00 | 42.82 | N |
| ATOM | 23239 | CA | THR | K | 219 | −5.344 | 69.389 | 67.638 | 1.00 | 38.39 | C |
| ATOM | 23241 | CB | THR | K | 219 | −4.217 | 68.450 | 67.188 | 1.00 | 40.90 | C |
| ATOM | 23243 | OG1 | THR | K | 219 | −2.964 | 69.164 | 67.193 | 1.00 | 34.21 | O |
| ATOM | 23245 | CG2 | THR | K | 219 | −4.386 | 68.026 | 65.726 | 1.00 | 27.12 | C |
| ATOM | 23249 | C | THR | K | 219 | −6.704 | 68.760 | 67.442 | 1.00 | 42.29 | C |
| ATOM | 23250 | O | THR | K | 219 | −7.390 | 69.044 | 66.438 | 1.00 | 47.53 | O |
| ATOM | 23251 | N | PRO | K | 220 | −7.142 | 67.980 | 68.429 | 1.00 | 43.42 | N |
| ATOM | 23252 | CA | PRO | K | 220 | −8.450 | 67.303 | 68.328 | 1.00 | 39.86 | C |
| ATOM | 23254 | CB | PRO | K | 220 | −8.536 | 66.434 | 69.589 | 1.00 | 36.43 | C |
| ATOM | 23257 | CG | PRO | K | 220 | −7.114 | 66.429 | 70.161 | 1.00 | 41.31 | C |
| ATOM | 23260 | CD | PRO | K | 220 | −6.460 | 67.710 | 69.706 | 1.00 | 37.01 | C |
| ATOM | 23263 | C | PRO | K | 220 | −9.595 | 68.278 | 68.284 | 1.00 | 38.15 | C |
| ATOM | 23264 | O | PRO | K | 220 | −10.666 | 67.825 | 67.944 | 1.00 | 55.71 | O |
| ATOM | 23265 | N | LEU | K | 221 | −9.406 | 69.545 | 68.642 | 1.00 | 44.54 | N |
| ATOM | 23267 | CA | LEU | K | 221 | −10.512 | 70.510 | 68.637 | 1.00 | 42.03 | C |
| ATOM | 23269 | CB | LEU | K | 221 | −10.129 | 71.819 | 69.293 | 1.00 | 31.62 | C |
| ATOM | 23272 | CG | LEU | K | 221 | −10.279 | 71.661 | 70.798 | 1.00 | 46.42 | C |
| ATOM | 23274 | CD1 | LEU | K | 221 | −9.611 | 72.774 | 71.533 | 1.00 | 53.79 | C |
| ATOM | 23278 | CD2 | LEU | K | 221 | −11.698 | 71.567 | 71.205 | 1.00 | 40.66 | C |
| ATOM | 23282 | C | LEU | K | 221 | −10.941 | 70.850 | 67.246 | 1.00 | 40.59 | C |
| ATOM | 23283 | O | LEU | K | 221 | −12.118 | 71.066 | 67.012 | 1.00 | 57.19 | O |
| ATOM | 23284 | N | SER | K | 222 | −9.991 | 70.955 | 66.335 | 1.00 | 41.97 | N |
| ATOM | 23286 | CA | SER | K | 222 | −10.302 | 71.375 | 64.973 | 1.00 | 42.44 | C |
| ATOM | 23288 | CB | SER | K | 222 | −10.258 | 72.897 | 64.885 | 1.00 | 48.40 | C |
| ATOM | 23291 | OG | SER | K | 222 | −9.509 | 73.387 | 63.782 | 1.00 | 62.98 | O |
| ATOM | 23293 | C | SER | K | 222 | −9.295 | 70.789 | 64.000 | 1.00 | 46.48 | C |
| ATOM | 23294 | O | SER | K | 222 | −8.104 | 70.635 | 64.311 | 1.00 | 52.88 | O |
| ATOM | 23295 | N | SER | K | 223 | −9.765 | 70.499 | 62.797 | 1.00 | 46.80 | N |
| ATOM | 23297 | CA | SER | K | 223 | −8.864 | 69.985 | 61.773 | 1.00 | 48.88 | C |
| ATOM | 23299 | CB | SER | K | 223 | −9.683 | 69.225 | 60.746 | 1.00 | 42.18 | C |
| ATOM | 23302 | OG | SER | K | 223 | −10.570 | 70.122 | 60.125 | 1.00 | 53.48 | O |
| ATOM | 23304 | C | SER | K | 223 | −7.927 | 71.016 | 61.102 | 1.00 | 48.12 | C |
| ATOM | 23305 | O | SER | K | 223 | −7.040 | 70.683 | 60.328 | 1.00 | 48.42 | O |
| ATOM | 23306 | N | THR | K | 224 | −8.078 | 72.283 | 61.425 | 1.00 | 54.00 | N |
| ATOM | 23308 | CA | THR | K | 224 | −7.242 | 73.279 | 60.797 | 1.00 | 56.03 | C |
| ATOM | 23310 | CB | THR | K | 224 | −7.973 | 73.922 | 59.571 | 1.00 | 56.06 | C |
| ATOM | 23312 | OG1 | THR | K | 224 | −7.878 | 75.348 | 59.624 | 1.00 | 63.18 | O |
| ATOM | 23314 | CG2 | THR | K | 224 | −9.476 | 73.708 | 59.634 | 1.00 | 68.18 | C |
| ATOM | 23318 | C | THR | K | 224 | −6.896 | 74.289 | 61.889 | 1.00 | 51.88 | C |
| ATOM | 23319 | O | THR | K | 224 | −7.629 | 74.472 | 62.862 | 1.00 | 55.88 | O |
| ATOM | 23320 | N | VAL | K | 225 | −5.753 | 74.932 | 61.711 | 1.00 | 43.00 | N |
| ATOM | 23322 | CA | VAL | K | 225 | −5.227 | 75.878 | 62.672 | 1.00 | 42.60 | C |
| ATOM | 23324 | CB | VAL | K | 225 | −4.040 | 75.300 | 63.482 | 1.00 | 44.50 | C |
| ATOM | 23326 | CG1 | VAL | K | 225 | −2.960 | 74.707 | 62.581 | 1.00 | 32.89 | C |
| ATOM | 23330 | CG2 | VAL | K | 225 | −3.449 | 76.391 | 64.334 | 1.00 | 48.05 | C |
| ATOM | 23334 | C | VAL | K | 225 | −4.707 | 77.060 | 61.897 | 1.00 | 39.29 | C |
| ATOM | 23335 | O | VAL | K | 225 | −4.232 | 76.892 | 60.773 | 1.00 | 39.10 | O |
| ATOM | 23336 | N | THR | K | 226 | −4.794 | 78.250 | 62.487 | 1.00 | 39.48 | N |
| ATOM | 23338 | CA | THR | K | 226 | −4.337 | 79.428 | 61.769 | 1.00 | 49.13 | C |
| ATOM | 23340 | CB | THR | K | 226 | −5.465 | 80.460 | 61.526 | 1.00 | 50.97 | C |
| ATOM | 23342 | OG1 | THR | K | 226 | −5.918 | 81.024 | 62.766 | 1.00 | 67.12 | O |
| ATOM | 23344 | CG2 | THR | K | 226 | −6.709 | 79.807 | 60.946 | 1.00 | 58.53 | C |
| ATOM | 23348 | C | THR | K | 226 | −3.181 | 80.025 | 62.552 | 1.00 | 46.08 | C |
| ATOM | 23349 | O | THR | K | 226 | −3.268 | 80.142 | 63.769 | 1.00 | 38.30 | O |
| ATOM | 23350 | N | LEU | K | 227 | −2.119 | 80.373 | 61.826 | 1.00 | 46.97 | N |
| ATOM | 23352 | CA | LEU | K | 227 | −0.949 | 81.061 | 62.388 | 1.00 | 54.07 | C |
| ATOM | 23354 | CB | LEU | K | 227 | 0.355 | 80.351 | 61.990 | 1.00 | 51.36 | C |
| ATOM | 23357 | CG | LEU | K | 227 | 0.281 | 78.839 | 62.116 | 1.00 | 44.55 | C |
| ATOM | 23359 | CD1 | LEU | K | 227 | 1.526 | 78.182 | 61.543 | 1.00 | 37.09 | C |
| ATOM | 23363 | CD2 | LEU | K | 227 | 0.083 | 78.453 | 63.571 | 1.00 | 51.90 | C |
| ATOM | 23367 | C | LEU | K | 227 | −0.845 | 82.527 | 61.968 | 1.00 | 46.47 | C |
| ATOM | 23368 | O | LEU | K | 227 | −0.743 | 82.837 | 60.786 | 1.00 | 49.71 | O |
| ATOM | 23369 | N | SER | K | 228 | −0.833 | 83.435 | 62.927 | 1.00 | 39.31 | N |
| ATOM | 23371 | CA | SER | K | 228 | −0.946 | 84.821 | 62.532 | 1.00 | 47.60 | C |
| ATOM | 23373 | CB | SER | K | 228 | −2.194 | 85.447 | 63.115 | 1.00 | 45.28 | C |
| ATOM | 23376 | OG | SER | K | 228 | −3.225 | 84.492 | 63.029 | 1.00 | 53.23 | O |
| ATOM | 23378 | C | SER | K | 228 | 0.257 | 85.457 | 63.120 | 1.00 | 48.31 | C |
| ATOM | 23379 | O | SER | K | 228 | 0.447 | 85.335 | 64.322 | 1.00 | 52.11 | O |
| ATOM | 23380 | N | MET | K | 229 | 1.106 | 86.039 | 62.281 | 1.00 | 43.95 | N |
| ATOM | 23382 | CA | MET | K | 229 | 2.377 | 86.462 | 62.803 | 1.00 | 52.86 | C |
| ATOM | 23384 | CB | MET | K | 229 | 3.394 | 85.338 | 62.660 | 1.00 | 55.87 | C |
| ATOM | 23387 | CG | MET | K | 229 | 3.682 | 84.876 | 61.268 | 1.00 | 58.88 | C |
| ATOM | 23390 | SD | MET | K | 229 | 4.553 | 83.311 | 61.420 | 1.00 | 66.53 | S |
| ATOM | 23391 | CE | MET | K | 229 | 3.864 | 82.528 | 60.006 | 1.00 | 65.34 | C |
| ATOM | 23395 | C | MET | K | 229 | 2.888 | 87.755 | 62.208 | 1.00 | 55.55 | C |
| ATOM | 23396 | O | MET | K | 229 | 2.545 | 88.086 | 61.087 | 1.00 | 54.05 | O |
| ATOM | 23397 | N | SER | K | 230 | 3.700 | 88.477 | 62.979 | 1.00 | 59.56 | N |
| ATOM | 23399 | CA | SER | K | 230 | 4.115 | 89.825 | 62.622 | 1.00 | 61.15 | C |
| ATOM | 23401 | CB | SER | K | 230 | 3.039 | 90.813 | 63.057 | 1.00 | 55.52 | C |
| ATOM | 23404 | OG | SER | K | 230 | 2.220 | 91.109 | 61.946 | 1.00 | 63.16 | O |
| ATOM | 23406 | C | SER | K | 230 | 5.427 | 90.135 | 63.316 | 1.00 | 62.52 | C |
| ATOM | 23407 | O | SER | K | 230 | 5.613 | 89.705 | 64.448 | 1.00 | 63.67 | O |
| ATOM | 23408 | N | ALA | K | 231 | 6.331 | 90.856 | 62.654 | 1.00 | 63.95 | N |
| ATOM | 23410 | CA | ALA | K | 231 | 7.557 | 91.326 | 63.304 | 1.00 | 61.38 | C |
| ATOM | 23412 | CB | ALA | K | 231 | 8.103 | 92.469 | 62.547 | 1.00 | 50.39 | C |
| ATOM | 23416 | C | ALA | K | 231 | 7.262 | 91.782 | 64.725 | 1.00 | 66.21 | C |
| ATOM | 23417 | O | ALA | K | 231 | 6.222 | 92.378 | 64.953 | 1.00 | 71.60 | O |
| ATOM | 23418 | N | ASP | K | 232 | 8.155 | 91.525 | 65.679 | 1.00 | 74.84 | N |
| ATOM | 23420 | CA | ASP | K | 232 | 8.095 | 92.166 | 67.011 | 1.00 | 79.91 | C |
| ATOM | 23422 | CB | ASP | K | 232 | 8.342 | 93.669 | 66.871 | 1.00 | 85.38 | C |
| ATOM | 23425 | CG | ASP | K | 232 | 9.787 | 94.051 | 67.058 | 1.00 | 95.67 | C |
| ATOM | 23426 | OD1 | ASP | K | 232 | 10.098 | 94.492 | 68.187 | 1.00 | 113.48 | O |
| ATOM | 23427 | OD2 | ASP | K | 232 | 10.632 | 94.003 | 66.129 | 1.00 | 85.88 | O |
| ATOM | 23428 | C | ASP | K | 232 | 6.757 | 92.005 | 67.743 | 1.00 | 74.23 | C |
| ATOM | 23429 | O | ASP | K | 232 | 6.209 | 92.964 | 68.299 | 1.00 | 69.60 | O |
| ATOM | 23430 | N | VAL | K | 233 | 6.198 | 90.805 | 67.726 | 1.00 | 65.31 | N |
| ATOM | 23432 | CA | VAL | K | 233 | 4.907 | 90.630 | 68.351 | 1.00 | 61.00 | C |
| ATOM | 23434 | CB | VAL | K | 233 | 3.817 | 91.418 | 67.618 | 1.00 | 62.20 | C |
| ATOM | 23436 | CG1 | VAL | K | 233 | 3.493 | 90.773 | 66.281 | 1.00 | 64.69 | C |
| ATOM | 23440 | CG2 | VAL | K | 233 | 2.564 | 91.500 | 68.482 | 1.00 | 77.95 | C |
| ATOM | 23444 | C | VAL | K | 233 | 4.592 | 89.154 | 68.357 | 1.00 | 53.13 | C |
| ATOM | 23445 | O | VAL | K | 233 | 5.049 | 88.410 | 67.498 | 1.00 | 62.57 | O |
| ATOM | 23446 | N | PRO | K | 234 | 3.934 | 88.696 | 69.406 | 1.00 | 44.95 | N |
| ATOM | 23447 | CA | PRO | K | 234 | 3.814 | 87.268 | 69.604 | 1.00 | 42.65 | C |
| ATOM | 23449 | CB | PRO | K | 234 | 3.166 | 87.151 | 70.985 | 1.00 | 45.57 | C |
| ATOM | 23452 | CG | PRO | K | 234 | 2.599 | 88.448 | 71.267 | 1.00 | 48.63 | C |
| ATOM | 23455 | CD | PRO | K | 234 | 3.448 | 89.443 | 70.572 | 1.00 | 53.02 | C |
| ATOM | 23458 | C | PRO | K | 234 | 3.005 | 86.614 | 68.501 | 1.00 | 46.19 | C |
| ATOM | 23459 | O | PRO | K | 234 | 2.118 | 87.211 | 67.890 | 1.00 | 36.24 | O |
| ATOM | 23460 | N | LEU | K | 235 | 3.351 | 85.348 | 68.283 | 1.00 | 46.45 | N |
| ATOM | 23462 | CA | LEU | K | 235 | 2.672 | 84.464 | 67.366 | 1.00 | 42.06 | C |
| ATOM | 23464 | CB | LEU | K | 235 | 3.481 | 83.182 | 67.311 | 1.00 | 37.82 | C |
| ATOM | 23467 | CG | LEU | K | 235 | 2.934 | 81.967 | 66.581 | 1.00 | 46.18 | C |
| ATOM | 23469 | CD1 | LEU | K | 235 | 2.809 | 82.233 | 65.111 | 1.00 | 39.24 | C |
| ATOM | 23473 | CD2 | LEU | K | 235 | 3.867 | 80.799 | 66.837 | 1.00 | 43.51 | C |
| ATOM | 23477 | C | LEU | K | 235 | 1.325 | 84.194 | 67.981 | 1.00 | 46.93 | C |
| ATOM | 23478 | O | LEU | K | 235 | 1.218 | 84.093 | 69.205 | 1.00 | 50.12 | O |
| ATOM | 23479 | N | VAL | K | 236 | 0.304 | 84.055 | 67.141 | 1.00 | 50.07 | N |
| ATOM | 23481 | CA | VAL | K | 236 | −0.956 | 83.458 | 67.570 | 1.00 | 54.11 | C |
| ATOM | 23483 | CB | VAL | K | 236 | −2.111 | 84.429 | 67.296 | 1.00 | 50.92 | C |
| ATOM | 23485 | CG1 | VAL | K | 236 | −3.396 | 83.930 | 67.921 | 1.00 | 58.63 | C |
| ATOM | 23489 | CG2 | VAL | K | 236 | −1.762 | 85.819 | 67.786 | 1.00 | 47.98 | C |
| ATOM | 23493 | C | VAL | K | 236 | −1.228 | 82.140 | 66.840 | 1.00 | 57.58 | C |
| ATOM | 23494 | O | VAL | K | 236 | −1.355 | 82.149 | 65.613 | 1.00 | 69.09 | O |
| ATOM | 23495 | N | VAL | K | 237 | −1.328 | 81.038 | 67.592 | 1.00 | 52.00 | N |
| ATOM | 23497 | CA | VAL | K | 237 | −1.874 | 79.756 | 67.117 | 1.00 | 42.50 | C |
| ATOM | 23499 | CB | VAL | K | 237 | −1.097 | 78.608 | 67.721 | 1.00 | 32.35 | C |
| ATOM | 23501 | CG1 | VAL | K | 237 | −1.594 | 77.313 | 67.177 | 1.00 | 34.06 | C |
| ATOM | 23505 | CG2 | VAL | K | 237 | 0.356 | 78.738 | 67.354 | 1.00 | 44.74 | C |
| ATOM | 23509 | C | VAL | K | 237 | −3.349 | 79.487 | 67.470 | 1.00 | 44.73 | C |
| ATOM | 23510 | O | VAL | K | 237 | −3.648 | 79.093 | 68.602 | 1.00 | 46.54 | O |
| ATOM | 23511 | N | GLU | K | 238 | −4.263 | 79.704 | 66.518 | 1.00 | 40.68 | N |
| ATOM | 23513 | CA | GLU | K | 238 | −5.683 | 79.716 | 66.818 | 1.00 | 37.38 | C |
| ATOM | 23515 | CB | GLU | K | 238 | −6.365 | 80.914 | 66.188 | 1.00 | 46.50 | C |
| ATOM | 23518 | CG | GLU | K | 238 | −7.898 | 80.896 | 66.224 | 1.00 | 54.21 | C |
| ATOM | 23521 | CD | GLU | K | 238 | −8.504 | 82.152 | 65.612 | 1.00 | 51.12 | C |
| ATOM | 23522 | OE1 | GLU | K | 238 | −7.998 | 82.620 | 64.573 | 1.00 | 60.93 | O |
| ATOM | 23523 | OE2 | GLU | K | 238 | −9.448 | 82.723 | 66.189 | 1.00 | 77.92 | O |
| ATOM | 23524 | C | GLU | K | 238 | −6.330 | 78.464 | 66.307 | 1.00 | 36.27 | C |
| ATOM | 23525 | O | GLU | K | 238 | −6.172 | 78.135 | 65.142 | 1.00 | 42.15 | O |
| ATOM | 23526 | N | TYR | K | 239 | −7.014 | 77.752 | 67.200 | 1.00 | 40.87 | N |
| ATOM | 23528 | CA | TYR | K | 239 | −7.843 | 76.604 | 66.855 | 1.00 | 41.32 | C |
| ATOM | 23530 | CB | TYR | K | 239 | −7.488 | 75.424 | 67.766 | 1.00 | 42.62 | C |
| ATOM | 23533 | CG | TYR | K | 239 | −6.070 | 74.944 | 67.643 | 1.00 | 40.44 | C |
| ATOM | 23534 | CD1 | TYR | K | 239 | −5.051 | 75.558 | 68.334 | 1.00 | 43.68 | C |
| ATOM | 23536 | CE1 | TYR | K | 239 | −3.753 | 75.129 | 68.231 | 1.00 | 31.03 | C |
| ATOM | 23538 | CZ | TYR | K | 239 | −3.456 | 74.069 | 67.449 | 1.00 | 29.84 | C |
| ATOM | 23539 | OH | TYR | K | 239 | −2.153 | 73.621 | 67.351 | 1.00 | 47.62 | O |
| ATOM | 23541 | CE2 | TYR | K | 239 | −4.468 | 73.411 | 66.797 | 1.00 | 52.62 | C |
| ATOM | 23543 | CD2 | TYR | K | 239 | −5.761 | 73.847 | 66.885 | 1.00 | 26.10 | C |
| ATOM | 23545 | C | TYR | K | 239 | −9.313 | 76.966 | 67.077 | 1.00 | 41.64 | C |
| ATOM | 23546 | O | TYR | K | 239 | −9.701 | 77.263 | 68.219 | 1.00 | 33.77 | O |
| ATOM | 23547 | N | LYS | K | 240 | −10.120 | 76.935 | 66.013 | 1.00 | 44.88 | N |
| ATOM | 23549 | CA | LYS | K | 240 | −11.565 | 77.186 | 66.152 | 1.00 | 49.14 | C |
| ATOM | 23551 | CB | LYS | K | 240 | −12.251 | 77.452 | 64.804 | 1.00 | 51.55 | C |
| ATOM | 23554 | CG | LYS | K | 240 | −11.920 | 78.825 | 64.124 | 1.00 | 72.80 | C |
| ATOM | 23557 | CD | LYS | K | 240 | −11.400 | 79.920 | 65.116 | 1.00 | 94.16 | C |
| ATOM | 23560 | CE | LYS | K | 240 | −12.248 | 81.224 | 65.171 | 1.00 | 93.98 | C |
| ATOM | 23563 | NZ | LYS | K | 240 | −12.435 | 81.854 | 66.541 | 1.00 | 76.50 | N |
| ATOM | 23567 | C | LYS | K | 240 | −12.240 | 76.020 | 66.851 | 1.00 | 44.73 | C |
| ATOM | 23568 | O | LYS | K | 240 | −12.127 | 74.896 | 66.411 | 1.00 | 45.16 | O |
| ATOM | 23569 | N | ILE | K | 241 | −12.947 | 76.286 | 67.946 | 1.00 | 53.25 | N |
| ATOM | 23571 | CA | ILE | K | 241 | −13.876 | 75.319 | 68.561 | 1.00 | 53.53 | C |
| ATOM | 23573 | CB | ILE | K | 241 | −13.965 | 75.593 | 70.073 | 1.00 | 47.47 | C |
| ATOM | 23575 | CG1 | ILE | K | 241 | −12.566 | 75.607 | 70.691 | 1.00 | 54.10 | C |
| ATOM | 23578 | CD1 | ILE | K | 241 | −12.501 | 76.045 | 72.159 | 1.00 | 47.86 | C |
| ATOM | 23582 | CG2 | ILE | K | 241 | −14.845 | 74.562 | 70.733 | 1.00 | 51.93 | C |
| ATOM | 23586 | C | ILE | K | 241 | −15.290 | 75.435 | 67.969 | 1.00 | 56.62 | C |
| ATOM | 23587 | O | ILE | K | 241 | −15.950 | 76.427 | 68.243 | 1.00 | 57.59 | O |
| ATOM | 23588 | N | ALA | K | 242 | −15.708 | 74.556 | 67.049 | 1.00 | 60.74 | N |
| ATOM | 23590 | CA | ALA | K | 242 | −16.520 | 75.040 | 65.921 | 1.00 | 58.91 | C |
| ATOM | 23592 | CB | ALA | K | 242 | −16.655 | 74.017 | 64.839 | 1.00 | 59.48 | C |
| ATOM | 23596 | C | ALA | K | 242 | −17.859 | 75.305 | 66.561 | 1.00 | 65.68 | C |
| ATOM | 23597 | O | ALA | K | 242 | −18.177 | 74.607 | 67.521 | 1.00 | 59.31 | O |
| ATOM | 23598 | N | ASP | K | 243 | −18.567 | 76.337 | 66.090 | 1.00 | 74.00 | N |
| ATOM | 23600 | CA | ASP | K | 243 | −19.664 | 77.019 | 66.812 | 1.00 | 79.18 | C |
| ATOM | 23602 | CB | ASP | K | 243 | −20.742 | 76.032 | 67.298 | 1.00 | 83.82 | C |
| ATOM | 23605 | CG | ASP | K | 243 | −21.711 | 75.633 | 66.191 | 1.00 | 93.91 | C |
| ATOM | 23606 | OD1 | ASP | K | 243 | −22.388 | 76.552 | 65.686 | 1.00 | 113.05 | O |
| ATOM | 23607 | OD2 | ASP | K | 243 | −21.850 | 74.461 | 65.750 | 1.00 | 85.65 | O |
| ATOM | 23608 | C | ASP | K | 243 | −19.228 | 77.909 | 67.979 | 1.00 | 77.46 | C |
| ATOM | 23609 | O | ASP | K | 243 | −19.123 | 79.136 | 67.827 | 1.00 | 79.76 | O |
| ATOM | 23610 | N | MET | K | 244 | −18.966 | 77.260 | 69.116 | 1.00 | 68.29 | N |
| ATOM | 23612 | CA | MET | K | 244 | −18.698 | 77.889 | 70.412 | 1.00 | 61.80 | C |
| ATOM | 23614 | CB | MET | K | 244 | −18.174 | 76.825 | 71.365 | 1.00 | 58.34 | C |
| ATOM | 23617 | CG | MET | K | 244 | −19.006 | 75.613 | 71.313 | 1.00 | 52.78 | C |
| ATOM | 23620 | SD | MET | K | 244 | −19.190 | 75.084 | 72.940 | 1.00 | 82.56 | S |
| ATOM | 23621 | CE | MET | K | 244 | −19.714 | 76.614 | 73.687 | 1.00 | 84.67 | C |
| ATOM | 23625 | C | MET | K | 244 | −17.723 | 79.062 | 70.488 | 1.00 | 57.35 | C |
| ATOM | 23626 | O | MET | K | 244 | −17.928 | 79.953 | 71.306 | 1.00 | 61.40 | O |
| ATOM | 23627 | N | GLY | K | 245 | −16.612 | 79.023 | 69.757 | 1.00 | 54.79 | N |
| ATOM | 23629 | CA | GLY | K | 245 | −15.475 | 79.880 | 70.078 | 1.00 | 56.11 | C |
| ATOM | 23632 | C | GLY | K | 245 | −14.106 | 79.414 | 69.628 | 1.00 | 51.14 | C |
| ATOM | 23633 | O | GLY | K | 245 | −13.896 | 78.996 | 68.500 | 1.00 | 58.97 | O |
| ATOM | 23634 | N | HIS | K | 246 | −13.117 | 79.506 | 70.491 | 1.00 | 52.90 | N |
| ATOM | 23636 | CA | HIS | K | 246 | −11.765 | 79.298 | 69.985 | 1.00 | 57.79 | C |
| ATOM | 23638 | CB | HIS | K | 246 | −11.400 | 80.472 | 69.072 | 1.00 | 49.92 | C |
| ATOM | 23641 | CG | HIS | K | 246 | −11.429 | 81.795 | 69.772 | 1.00 | 68.63 | C |
| ATOM | 23642 | ND1 | HIS | K | 246 | −12.360 | 82.773 | 69.484 | 1.00 | 68.69 | N |
| ATOM | 23644 | CE1 | HIS | K | 246 | −12.143 | 83.817 | 70.267 | 1.00 | 72.19 | C |
| ATOM | 23646 | NE2 | HIS | K | 246 | −11.112 | 83.552 | 71.052 | 1.00 | 70.06 | N |
| ATOM | 23648 | CD2 | HIS | K | 246 | −10.661 | 82.284 | 70.778 | 1.00 | 68.47 | C |
| ATOM | 23650 | C | HIS | K | 246 | −10.750 | 79.134 | 71.127 | 1.00 | 55.82 | C |
| ATOM | 23651 | O | HIS | K | 246 | −10.936 | 79.700 | 72.204 | 1.00 | 59.60 | O |
| ATOM | 23652 | N | LEU | K | 247 | −9.687 | 78.361 | 70.907 | 1.00 | 49.07 | N |
| ATOM | 23654 | CA | LEU | K | 247 | −8.510 | 78.445 | 71.771 | 1.00 | 42.95 | C |
| ATOM | 23656 | CB | LEU | K | 247 | −8.063 | 77.057 | 72.163 | 1.00 | 42.87 | C |
| ATOM | 23659 | CG | LEU | K | 247 | −7.518 | 76.840 | 73.563 | 1.00 | 55.82 | C |
| ATOM | 23661 | CD1 | LEU | K | 247 | −8.107 | 77.778 | 74.595 | 1.00 | 57.76 | C |
| ATOM | 23665 | CD2 | LEU | K | 247 | −7.725 | 75.390 | 73.961 | 1.00 | 51.52 | C |
| ATOM | 23669 | C | LEU | K | 247 | −7.368 | 79.128 | 71.035 | 1.00 | 49.81 | C |
| ATOM | 23670 | O | LEU | K | 247 | −6.927 | 78.703 | 69.965 | 1.00 | 47.65 | O |
| ATOM | 23671 | N | LYS | K | 248 | −6.884 | 80.218 | 71.600 | 1.00 | 47.38 | N |
| ATOM | 23673 | CA | LYS | K | 248 | −5.850 | 80.955 | 70.914 | 1.00 | 44.46 | C |
| ATOM | 23675 | CB | LYS | K | 248 | −6.271 | 82.419 | 70.786 | 1.00 | 45.26 | C |
| ATOM | 23678 | CG | LYS | K | 248 | −7.049 | 82.724 | 69.516 | 1.00 | 49.28 | C |
| ATOM | 23681 | CD | LYS | K | 248 | −7.883 | 84.014 | 69.637 | 1.00 | 61.15 | C |
| ATOM | 23684 | CE | LYS | K | 248 | −8.601 | 84.414 | 68.340 | 1.00 | 43.95 | C |
| ATOM | 23687 | NZ | LYS | K | 248 | −8.292 | 85.841 | 68.086 | 1.00 | 63.47 | N |
| ATOM | 23691 | C | LYS | K | 248 | −4.624 | 80.833 | 71.785 | 1.00 | 41.81 | C |
| ATOM | 23692 | O | LYS | K | 248 | −4.702 | 80.964 | 73.002 | 1.00 | 44.40 | O |
| ATOM | 23693 | N | TYR | K | 249 | −3.481 | 80.576 | 71.187 | 1.00 | 41.17 | N |
| ATOM | 23695 | CA | TYR | K | 249 | −2.275 | 80.468 | 71.999 | 1.00 | 49.54 | C |
| ATOM | 23697 | CB | TYR | K | 249 | −1.639 | 79.111 | 71.758 | 1.00 | 46.94 | C |
| ATOM | 23700 | CG | TYR | K | 249 | −2.432 | 77.949 | 72.290 | 1.00 | 47.20 | C |
| ATOM | 23701 | CD1 | TYR | K | 249 | −2.257 | 77.510 | 73.589 | 1.00 | 47.12 | C |
| ATOM | 23703 | CE1 | TYR | K | 249 | −2.911 | 76.380 | 74.054 | 1.00 | 49.92 | C |
| ATOM | 23705 | CZ | TYR | K | 249 | −3.771 | 75.679 | 73.231 | 1.00 | 55.09 | C |
| ATOM | 23706 | OH | TYR | K | 249 | −4.423 | 74.561 | 73.728 | 1.00 | 40.54 | O |
| ATOM | 23708 | CE2 | TYR | K | 249 | −3.965 | 76.102 | 71.925 | 1.00 | 47.05 | C |
| ATOM | 23710 | CD2 | TYR | K | 249 | −3.287 | 77.225 | 71.468 | 1.00 | 49.09 | C |
| ATOM | 23712 | C | TYR | K | 249 | −1.268 | 81.541 | 71.618 | 1.00 | 47.02 | C |
| ATOM | 23713 | O | TYR | K | 249 | −0.839 | 81.581 | 70.474 | 1.00 | 46.16 | O |
| ATOM | 23714 | N | TYR | K | 250 | −0.888 | 82.424 | 72.534 | 1.00 | 44.44 | N |
| ATOM | 23716 | CA | TYR | K | 250 | 0.088 | 83.451 | 72.154 | 1.00 | 44.40 | C |
| ATOM | 23718 | CB | TYR | K | 250 | −0.268 | 84.850 | 72.675 | 1.00 | 38.55 | C |
| ATOM | 23721 | CG | TYR | K | 250 | −1.589 | 85.386 | 72.161 | 1.00 | 41.69 | C |
| ATOM | 23722 | CD1 | TYR | K | 250 | −2.805 | 84.972 | 72.704 | 1.00 | 54.74 | C |
| ATOM | 23724 | CE1 | TYR | K | 250 | −4.018 | 85.462 | 72.240 | 1.00 | 38.56 | C |
| ATOM | 23726 | CZ | TYR | K | 250 | −4.013 | 86.356 | 71.207 | 1.00 | 47.96 | C |
| ATOM | 23727 | OH | TYR | K | 250 | −5.202 | 86.816 | 70.679 | 1.00 | 35.13 | O |
| ATOM | 23729 | CE2 | TYR | K | 250 | −2.814 | 86.788 | 70.678 | 1.00 | 38.55 | C |
| ATOM | 23731 | CD2 | TYR | K | 250 | −1.626 | 86.339 | 71.178 | 1.00 | 27.82 | C |
| ATOM | 23733 | C | TYR | K | 250 | 1.461 | 83.030 | 72.642 | 1.00 | 43.55 | C |
| ATOM | 23734 | O | TYR | K | 250 | 1.630 | 82.602 | 73.782 | 1.00 | 44.22 | O |
| ATOM | 23735 | N | LEU | K | 251 | 2.449 | 83.196 | 71.777 | 1.00 | 47.59 | N |
| ATOM | 23737 | CA | LEU | K | 251 | 3.820 | 82.808 | 72.096 | 1.00 | 51.11 | C |
| ATOM | 23739 | CB | LEU | K | 251 | 4.236 | 81.602 | 71.273 | 1.00 | 50.95 | C |
| ATOM | 23742 | CG | LEU | K | 251 | 5.665 | 81.129 | 71.466 | 1.00 | 46.87 | C |
| ATOM | 23744 | CD1 | LEU | K | 251 | 5.940 | 80.837 | 72.939 | 1.00 | 43.60 | C |
| ATOM | 23748 | CD2 | LEU | K | 251 | 5.766 | 79.866 | 70.653 | 1.00 | 59.34 | C |
| ATOM | 23752 | C | LEU | K | 251 | 4.773 | 83.933 | 71.761 | 1.00 | 51.43 | C |
| ATOM | 23753 | O | LEU | K | 251 | 4.768 | 84.441 | 70.644 | 1.00 | 58.37 | O |
| ATOM | 23754 | N | ALA | K | 252 | 5.594 | 84.311 | 72.727 | 1.00 | 47.15 | N |
| ATOM | 23756 | CA | ALA | K | 252 | 6.585 | 85.355 | 72.503 | 1.00 | 49.29 | C |
| ATOM | 23758 | CB | ALA | K | 252 | 7.085 | 85.837 | 73.856 | 1.00 | 55.54 | C |
| ATOM | 23762 | C | ALA | K | 252 | 7.772 | 84.839 | 71.677 | 1.00 | 47.57 | C |
| ATOM | 23763 | O | ALA | K | 252 | 8.298 | 83.759 | 71.939 | 1.00 | 48.35 | O |
| ATOM | 23764 | N | PRO | K | 253 | 8.231 | 85.624 | 70.708 | 1.00 | 51.50 | N |
| ATOM | 23765 | CA | PRO | K | 253 | 9.368 | 85.231 | 69.866 | 1.00 | 50.46 | C |
| ATOM | 23767 | CB | PRO | K | 253 | 9.436 | 86.325 | 68.818 | 1.00 | 43.59 | C |
| ATOM | 23770 | CG | PRO | K | 253 | 8.785 | 87.492 | 69.454 | 1.00 | 57.24 | C |
| ATOM | 23773 | CD | PRO | K | 253 | 7.719 | 86.956 | 70.358 | 1.00 | 53.10 | C |
| ATOM | 23776 | C | PRO | K | 253 | 10.681 | 85.134 | 70.622 | 1.00 | 49.12 | C |
| ATOM | 23777 | O | PRO | K | 253 | 10.838 | 85.696 | 71.706 | 1.00 | 44.36 | O |
| ATOM | 23778 | N | LYS | K | 254 | 11.589 | 84.332 | 70.077 | 1.00 | 53.66 | N |
| ATOM | 23780 | CA | LYS | K | 254 | 13.027 | 84.606 | 70.184 | 1.00 | 57.27 | C |
| ATOM | 23782 | CB | LYS | K | 254 | 13.771 | 83.315 | 69.969 | 1.00 | 49.15 | C |
| ATOM | 23785 | CG | LYS | K | 254 | 13.583 | 82.413 | 71.117 | 1.00 | 36.13 | C |
| ATOM | 23788 | CD | LYS | K | 254 | 14.646 | 81.300 | 71.131 | 1.00 | 56.21 | C |
| ATOM | 23791 | CE | LYS | K | 254 | 14.418 | 80.296 | 72.295 | 1.00 | 77.55 | C |
| ATOM | 23794 | NZ | LYS | K | 254 | 15.030 | 78.955 | 72.021 | 1.00 | 74.11 | N |
| ATOM | 23798 | C | LYS | K | 254 | 13.571 | 85.643 | 69.197 | 1.00 | 58.37 | C |
| ATOM | 23799 | O | LYS | K | 254 | 13.352 | 85.496 | 67.988 | 1.00 | 58.91 | O |
| ATOM | 23800 | N | ILE | K | 255 | 14.233 | 86.684 | 69.706 | 1.00 | 58.07 | N |
| ATOM | 23802 | CA | ILE | K | 255 | 14.739 | 87.746 | 68.845 | 1.00 | 67.78 | C |
| ATOM | 23804 | CB | ILE | K | 255 | 14.014 | 89.059 | 69.115 | 1.00 | 71.49 | C |
| ATOM | 23806 | CG1 | ILE | K | 255 | 14.089 | 89.382 | 70.599 | 1.00 | 84.31 | C |
| ATOM | 23809 | CD1 | ILE | K | 255 | 13.818 | 90.835 | 70.889 | 1.00 | 92.91 | C |
| ATOM | 23813 | CG2 | ILE | K | 255 | 12.577 | 89.030 | 68.582 | 1.00 | 74.11 | C |
| ATOM | 23817 | C | ILE | K | 255 | 18.235 | 87.897 | 69.079 | 1.00 | 71.89 | C |
| ATOM | 23818 | O | ILE | K | 255 | 18.755 | 87.224 | 69.959 | 1.00 | 73.90 | O |
| ATOM | 23819 | N | GLU | K | 256 | 16.935 | 88.686 | 68.256 | 1.00 | 84.63 | N |
| ATOM | 23821 | CA | GLU | K | 256 | 18.414 | 88.746 | 68.275 | 1.00 | 93.55 | C |
| ATOM | 23823 | CB | GLU | K | 256 | 18.999 | 89.017 | 66.878 | 1.00 | 92.17 | C |
| ATOM | 23826 | CG | GLU | K | 256 | 20.356 | 88.341 | 66.664 | 1.00 | 107.04 | C |
| ATOM | 23829 | CD | GLU | K | 256 | 21.564 | 89.282 | 66.578 | 1.00 | 120.41 | C |
| ATOM | 23830 | OE1 | GLU | K | 256 | 21.583 | 90.364 | 67.219 | 1.00 | 113.29 | O |
| ATOM | 23831 | OE2 | GLU | K | 256 | 22.537 | 88.914 | 65.874 | 1.00 | 116.82 | O |
| ATOM | 23832 | C | GLU | K | 256 | 18.980 | 89.799 | 69.236 | 1.00 | 95.81 | C |
| ATOM | 23833 | O | GLU | K | 256 | 18.533 | 90.947 | 69.209 | 1.00 | 95.41 | O |
| ATOM | 23834 | N | ASP | K | 257 | 19.988 | 89.433 | 70.035 | 1.00 | 99.38 | N |
| ATOM | 23836 | CA | ASP | K | 257 | 20.869 | 90.424 | 70.680 | 1.00 | 102.59 | C |
| ATOM | 23838 | CB | ASP | K | 257 | 20.087 | 91.305 | 71.671 | 1.00 | 103.81 | C |
| ATOM | 23841 | CG | ASP | K | 257 | 20.739 | 92.660 | 71.896 | 1.00 | 110.04 | C |
| ATOM | 23842 | OD1 | ASP | K | 257 | 20.064 | 93.692 | 71.698 | 1.00 | 106.01 | O |
| ATOM | 23843 | OD2 | ASP | K | 257 | 21.919 | 92.796 | 72.286 | 1.00 | 125.66 | O |
| ATOM | 23844 | C | ASP | K | 257 | 22.077 | 89.808 | 71.393 | 1.00 | 99.60 | C |
| ATOM | 23845 | O | ASP | K | 257 | 22.122 | 88.606 | 71.665 | 1.00 | 95.52 | O |
| ATOM | 23846 | N | SER | B | 1 | −30.411 | 49.909 | 41.934 | 1.00 | 154.07 | N |
| ATOM | 23848 | CA | SER | B | 1 | −31.361 | 51.050 | 41.786 | 1.00 | 153.63 | C |
| ATOM | 23850 | CB | SER | B | 1 | −32.794 | 50.575 | 42.057 | 1.00 | 153.77 | C |
| ATOM | 23853 | OG | SER | B | 1 | −32.860 | 49.155 | 42.100 | 1.00 | 149.73 | O |
| ATOM | 23855 | C | SER | B | 1 | −30.991 | 52.188 | 42.739 | 1.00 | 153.34 | C |
| ATOM | 23856 | O | SER | B | 1 | −29.977 | 52.115 | 43.437 | 1.00 | 150.59 | O |
| ATOM | 23859 | N | ALA | B | 2 | −31.808 | 53.241 | 42.750 | 1.00 | 153.70 | N |
| ATOM | 23861 | CA | ALA | B | 2 | −32.104 | 54.000 | 43.971 | 1.00 | 152.23 | C |
| ATOM | 23863 | CB | ALA | B | 2 | −31.115 | 55.173 | 44.136 | 1.00 | 151.55 | C |
| ATOM | 23867 | C | ALA | B | 2 | −33.557 | 54.505 | 44.082 | 1.00 | 148.13 | C |
| ATOM | 23868 | O | ALA | B | 2 | −33.951 | 54.977 | 45.156 | 1.00 | 148.86 | O |
| ATOM | 23869 | N | VAL | B | 3 | −34.344 | 54.400 | 43.005 | 1.00 | 140.08 | N |
| ATOM | 23871 | CA | VAL | B | 3 | −35.281 | 55.461 | 42.619 | 1.00 | 133.44 | C |
| ATOM | 23873 | CB | VAL | B | 3 | −34.546 | 56.817 | 42.521 | 1.00 | 135.47 | C |
| ATOM | 23875 | CG1 | VAL | B | 3 | −34.176 | 57.362 | 43.905 | 1.00 | 139.90 | C |
| ATOM | 23879 | CG2 | VAL | B | 3 | −33.301 | 56.686 | 41.663 | 1.00 | 138.99 | C |
| ATOM | 23883 | C | VAL | B | 3 | −36.034 | 55.237 | 41.295 | 1.00 | 124.68 | C |
| ATOM | 23884 | O | VAL | B | 3 | −35.921 | 54.183 | 40.663 | 1.00 | 119.60 | O |
| ATOM | 23885 | N | LEU | B | 4 | −36.796 | 56.252 | 40.882 | 1.00 | 117.86 | N |
| ATOM | 23887 | CA | LEU | B | 4 | −37.912 | 56.099 | 39.930 | 1.00 | 114.26 | C |
| ATOM | 23889 | CB | LEU | B | 4 | −39.184 | 55.577 | 40.631 | 1.00 | 117.89 | C |
| ATOM | 23892 | CG | LEU | B | 4 | −39.076 | 54.384 | 41.608 | 1.00 | 131.05 | C |
| ATOM | 23894 | CD1 | LEU | B | 4 | −40.369 | 54.137 | 42.428 | 1.00 | 130.11 | C |
| ATOM | 23898 | CD2 | LEU | B | 4 | −38.649 | 53.100 | 40.888 | 1.00 | 133.23 | C |
| ATOM | 23902 | C | LEU | B | 4 | −38.230 | 57.394 | 39.150 | 1.00 | 99.61 | C |
| ATOM | 23903 | O | LEU | B | 4 | −38.275 | 58.491 | 39.720 | 1.00 | 90.50 | O |
| ATOM | 23904 | N | GLN | B | 5 | −38.448 | 57.247 | 37.842 | 1.00 | 86.41 | N |
| ATOM | 23906 | CA | GLN | B | 5 | −38.195 | 58.307 | 36.857 | 1.00 | 74.64 | C |
| ATOM | 23908 | CB | GLN | B | 5 | −37.821 | 57.713 | 35.491 | 1.00 | 78.64 | C |
| ATOM | 23911 | CG | GLN | B | 5 | −37.731 | 58.719 | 34.303 | 1.00 | 65.12 | C |
| ATOM | 23914 | CD | GLN | B | 5 | −36.537 | 59.662 | 34.388 | 1.00 | 55.62 | C |
| ATOM | 23915 | OE1 | GLN | B | 5 | −35.416 | 59.289 | 34.046 | 1.00 | 52.40 | O |
| ATOM | 23916 | NE2 | GLN | B | 5 | −36.759 | 60.854 | 34.925 | 1.00 | 60.29 | N |
| ATOM | 23919 | C | GLN | B | 5 | −39.428 | 59.156 | 36.632 | 1.00 | 72.16 | C |
| ATOM | 23920 | O | GLN | B | 5 | −40.402 | 58.727 | 36.009 | 1.00 | 69.74 | O |
| ATOM | 23921 | N | LYS | B | 6 | −39.367 | 60.403 | 37.061 | 1.00 | 70.29 | N |
| ATOM | 23923 | CA | LYS | B | 6 | −40.479 | 61.296 | 36.796 | 1.00 | 65.77 | C |
| ATOM | 23925 | CB | LYS | B | 6 | −40.355 | 62.524 | 37.699 | 1.00 | 68.60 | C |
| ATOM | 23928 | CG | LYS | B | 6 | −40.793 | 62.192 | 39.144 | 1.00 | 70.02 | C |
| ATOM | 23931 | CD | LYS | B | 6 | −42.177 | 61.482 | 39.182 | 1.00 | 83.37 | C |
| ATOM | 23934 | CE | LYS | B | 6 | −43.132 | 62.044 | 40.240 | 1.00 | 88.41 | C |
| ATOM | 23937 | NZ | LYS | B | 6 | −43.838 | 63.273 | 39.782 | 1.00 | 84.45 | N |
| ATOM | 23941 | C | LYS | B | 6 | −40.638 | 61.609 | 35.301 | 1.00 | 64.03 | C |
| ATOM | 23942 | O | LYS | B | 6 | −39.749 | 61.324 | 34.494 | 1.00 | 60.79 | O |
| ATOM | 23943 | N | LYS | B | 7 | −41.808 | 62.103 | 34.902 | 1.00 | 64.49 | N |
| ATOM | 23945 | CA | LYS | B | 7 | −41.972 | 62.621 | 33.547 | 1.00 | 59.63 | C |
| ATOM | 23947 | CB | LYS | B | 7 | −43.259 | 62.117 | 32.922 | 1.00 | 53.76 | C |
| ATOM | 23950 | CG | LYS | B | 7 | −43.588 | 60.701 | 33.299 | 1.00 | 74.30 | C |
| ATOM | 23953 | CD | LYS | B | 7 | −43.989 | 59.865 | 32.086 | 1.00 | 82.35 | C |
| ATOM | 23956 | CE | LYS | B | 7 | −45.495 | 59.883 | 31.875 | 1.00 | 75.04 | C |
| ATOM | 23959 | NZ | LYS | B | 7 | −46.019 | 58.517 | 31.614 | 1.00 | 65.80 | N |
| ATOM | 23963 | C | LYS | B | 7 | −41.915 | 64.144 | 33.499 | 1.00 | 57.74 | C |
| ATOM | 23964 | O | LYS | B | 7 | −41.975 | 64.825 | 34.522 | 1.00 | 55.38 | O |
| ATOM | 23965 | N | ILE | B | 8 | −41.740 | 64.704 | 32.312 | 1.00 | 56.60 | N |
| ATOM | 23967 | CA | ILE | B | 8 | −41.589 | 66.146 | 32.280 | 1.00 | 62.25 | C |
| ATOM | 23969 | CB | ILE | B | 8 | −40.875 | 66.606 | 31.000 | 1.00 | 60.71 | C |
| ATOM | 23971 | CG1 | ILE | B | 8 | −40.450 | 68.050 | 31.159 | 1.00 | 55.25 | C |
| ATOM | 23974 | CD1 | ILE | B | 8 | −38.953 | 68.098 | 31.392 | 1.00 | 77.67 | C |
| ATOM | 23978 | CG2 | ILE | B | 8 | −41.724 | 66.427 | 29.767 | 1.00 | 61.95 | C |
| ATOM | 23982 | C | ILE | B | 8 | −42.962 | 66.792 | 32.490 | 1.00 | 59.20 | C |
| ATOM | 23983 | O | ILE | B | 8 | −43.075 | 67.869 | 33.059 | 1.00 | 60.42 | O |
| ATOM | 23984 | N | THR | B | 9 | −44.002 | 66.066 | 32.103 | 1.00 | 54.68 | N |
| ATOM | 23986 | CA | THR | B | 9 | −45.384 | 66.448 | 32.365 | 1.00 | 52.68 | C |
| ATOM | 23988 | CB | THR | B | 9 | −46.363 | 65.447 | 31.739 | 1.00 | 43.70 | C |
| ATOM | 23990 | OG1 | THR | B | 9 | −46.171 | 64.150 | 32.313 | 1.00 | 58.65 | O |
| ATOM | 23992 | CG2 | THR | B | 9 | −46.079 | 65.204 | 30.256 | 1.00 | 47.81 | C |
| ATOM | 23996 | C | THR | B | 9 | −45.753 | 66.590 | 33.833 | 1.00 | 57.05 | C |
| ATOM | 23997 | O | THR | B | 9 | −46.648 | 67.374 | 34.131 | 1.00 | 76.07 | O |
| ATOM | 23998 | N | ASP | B | 10 | −45.133 | 65.869 | 34.765 | 1.00 | 57.69 | N |
| ATOM | 24000 | CA | ASP | B | 10 | −45.431 | 66.115 | 36.192 | 1.00 | 54.11 | C |
| ATOM | 24002 | CB | ASP | B | 10 | −44.898 | 66.021 | 37.135 | 1.00 | 46.67 | C |
| ATOM | 24005 | CG | ASP | B | 10 | −44.984 | 63.619 | 36.537 | 1.00 | 54.38 | C |
| ATOM | 24006 | OD1 | ASP | B | 10 | −46.052 | 63.255 | 36.013 | 1.00 | 51.58 | O |
| ATOM | 24007 | OD2 | ASP | B | 10 | −44.054 | 62.778 | 36.557 | 1.00 | 60.59 | O |
| ATOM | 24008 | C | ASP | B | 10 | −44.967 | 67.483 | 36.691 | 1.00 | 54.14 | C |
| ATOM | 24009 | O | ASP | B | 10 | −45.066 | 67.740 | 37.876 | 1.00 | 63.14 | O |
| ATOM | 24010 | N | TYR | B | 11 | −44.426 | 68.335 | 35.824 | 1.00 | 59.87 | N |
| ATOM | 24012 | CA | TYR | B | 11 | −43.767 | 69.576 | 36.253 | 1.00 | 62.03 | C |
| ATOM | 24014 | CB | TYR | B | 11 | −42.223 | 69.507 | 36.212 | 1.00 | 60.60 | C |
| ATOM | 24017 | CG | TYR | B | 11 | −41.543 | 68.454 | 37.079 | 1.00 | 65.71 | C |
| ATOM | 24018 | CD1 | TYR | B | 11 | −41.429 | 67.118 | 36.664 | 1.00 | 62.77 | C |
| ATOM | 24020 | CE1 | TYR | B | 11 | −40.788 | 66.150 | 37.480 | 1.00 | 48.58 | C |
| ATOM | 24022 | CZ | TYR | B | 11 | −40.243 | 66.547 | 38.689 | 1.00 | 49.87 | C |
| ATOM | 24023 | OH | TYR | B | 11 | −39.643 | 65.652 | 39.528 | 1.00 | 58.39 | O |
| ATOM | 24025 | CE2 | TYR | B | 11 | −40.296 | 67.869 | 39.087 | 1.00 | 68.39 | C |
| ATOM | 24027 | CD2 | TYR | B | 11 | −40.923 | 68.816 | 38.273 | 1.00 | 73.89 | C |
| ATOM | 24029 | C | TYR | B | 11 | −44.186 | 70.714 | 35.332 | 1.00 | 63.00 | C |
| ATOM | 24030 | O | TYR | B | 11 | −44.194 | 71.865 | 35.771 | 1.00 | 65.01 | O |
| ATOM | 24031 | N | PHE | B | 12 | −44.450 | 70.409 | 34.059 | 1.00 | 60.69 | N |
| ATOM | 24033 | CA | PHE | B | 12 | −44.800 | 71.425 | 33.061 | 1.00 | 66.26 | C |
| ATOM | 24035 | CB | PHE | B | 12 | −43.802 | 71.526 | 31.888 | 1.00 | 68.93 | C |
| ATOM | 24038 | CG | PHE | B | 12 | −42.412 | 71.948 | 32.295 | 1.00 | 69.65 | C |
| ATOM | 24039 | CD1 | PHE | B | 12 | −41.300 | 71.500 | 31.603 | 1.00 | 74.65 | C |
| ATOM | 24041 | CE1 | PHE | B | 12 | −40.035 | 71.882 | 31.984 | 1.00 | 79.56 | C |
| ATOM | 24043 | CZ | PHE | B | 12 | −39.855 | 72.684 | 33.091 | 1.00 | 71.71 | C |
| ATOM | 24045 | CE2 | PHE | B | 12 | −40.938 | 73.123 | 33.787 | 1.00 | 67.45 | C |
| ATOM | 24047 | CD2 | PHE | B | 12 | −42.215 | 72.760 | 33.388 | 1.00 | 50.12 | C |
| ATOM | 24049 | C | PHE | B | 12 | −46.144 | 71.069 | 32.468 | 1.00 | 70.01 | C |
| ATOM | 24050 | O | PHE | B | 12 | −46.388 | 69.914 | 32.092 | 1.00 | 69.44 | O |
| ATOM | 24051 | N | HIS | B | 13 | −47.022 | 72.063 | 32.376 | 1.00 | 72.64 | N |
| ATOM | 24053 | CA | HIS | B | 13 | −48.418 | 71.750 | 32.105 | 1.00 | 70.73 | C |
| ATOM | 24055 | CB | HIS | B | 13 | −49.255 | 72.022 | 33.365 | 1.00 | 75.39 | C |
| ATOM | 24058 | CG | HIS | B | 13 | −48.992 | 71.067 | 34.502 | 1.00 | 84.45 | C |
| ATOM | 24059 | ND1 | HIS | B | 13 | −48.613 | 89.752 | 34.315 | 1.00 | 83.05 | N |
| ATOM | 24061 | CE1 | HIS | B | 13 | −48.464 | 69.161 | 35.487 | 1.00 | 78.39 | C |
| ATOM | 24063 | NE2 | HIS | B | 13 | −48.758 | 70.036 | 36.432 | 1.00 | 91.51 | N |
| ATOM | 24065 | CD2 | HIS | B | 13 | −49.101 | 71.231 | 35.844 | 1.00 | 91.77 | C |
| ATOM | 24067 | C | HIS | B | 13 | −48.834 | 72.578 | 30.899 | 1.00 | 64.54 | C |
| ATOM | 24068 | O | HIS | B | 13 | −48.190 | 73.586 | 30.607 | 1.00 | 62.02 | O |
| ATOM | 24069 | N | PRO | B | 14 | −49.841 | 72.122 | 30.159 | 1.00 | 60.40 | N |
| ATOM | 24070 | CA | PRO | B | 14 | −50.257 | 72.798 | 28.927 | 1.00 | 65.77 | C |
| ATOM | 24072 | CB | PRO | B | 14 | −51.518 | 72.037 | 28.489 | 1.00 | 59.38 | C |
| ATOM | 24075 | CG | PRO | B | 14 | −51.432 | 70.738 | 29.105 | 1.00 | 50.31 | C |
| ATOM | 24078 | CD | PRO | B | 14 | −50.661 | 70.932 | 30.415 | 1.00 | 60.58 | C |
| ATOM | 24081 | C | PRO | B | 14 | −50.595 | 74.262 | 29.145 | 1.00 | 72.97 | C |
| ATOM | 24082 | O | PRO | B | 14 | −50.876 | 74.672 | 30.268 | 1.00 | 67.31 | O |
| ATOM | 24083 | N | LYS | B | 15 | −50.541 | 75.043 | 28.073 | 1.00 | 87.59 | N |
| ATOM | 24085 | CA | LYS | B | 15 | −50.408 | 76.482 | 28.218 | 1.00 | 98.92 | C |
| ATOM | 24087 | CB | LYS | B | 15 | −49.863 | 77.145 | 26.946 | 1.00 | 100.51 | C |
| ATOM | 24090 | CG | LYS | B | 15 | −48.484 | 77.804 | 27.117 | 1.00 | 103.30 | C |
| ATOM | 24093 | CD | LYS | B | 15 | −48.561 | 79.253 | 27.622 | 1.00 | 106.09 | C |
| ATOM | 24096 | CE | LYS | B | 15 | −48.645 | 80.248 | 26.464 | 1.00 | 102.61 | C |
| ATOM | 24099 | NZ | LYS | B | 15 | −47.505 | 81.197 | 26.436 | 1.00 | 90.48 | N |
| ATOM | 24103 | C | LYS | B | 15 | −51.777 | 77.025 | 28.618 | 1.00 | 109.07 | C |
| ATOM | 24104 | O | LYS | B | 15 | −52.706 | 77.107 | 27.804 | 1.00 | 110.25 | O |
| ATOM | 24105 | N | LYS | B | 16 | −51.904 | 77.294 | 29.917 | 1.00 | 119.24 | N |
| ATOM | 24107 | CA | LYS | B | 16 | −52.551 | 78.506 | 30.419 | 1.00 | 121.90 | C |
| ATOM | 24109 | CB | LYS | B | 16 | −51.487 | 79.549 | 30.835 | 1.00 | 122.89 | C |
| ATOM | 24112 | CG | LYS | B | 16 | −51.165 | 79.602 | 32.357 | 1.00 | 125.60 | C |
| ATOM | 24115 | CD | LYS | B | 16 | −49.981 | 78.708 | 32.855 | 1.00 | 136.34 | C |
| ATOM | 24118 | CE | LYS | B | 16 | −48.656 | 78.842 | 32.057 | 1.00 | 141.85 | C |
| ATOM | 24121 | NZ | LYS | B | 16 | −48.700 | 79.909 | 30.983 | 1.00 | 150.18 | N |
| ATOM | 24125 | C | LYS | B | 16 | −53.519 | 78.997 | 29.328 | 1.00 | 121.36 | C |
| ATOM | 24126 | O | LYS | B | 16 | −54.630 | 78.476 | 29.207 | 1.00 | 122.70 | O |
| ATOM | 24127 | OXT | LYS | B | 16 | −53.287 | 79.832 | 28.446 | 1.00 | 115.35 | O |
| ATOM | 24128 | N | SER | D | 1 | −37.526 | 58.915 | −17.680 | 1.00 | 122.29 | N |
| ATOM | 24130 | CA | SER | D | 1 | −37.829 | 59.127 | −16.231 | 1.00 | 123.68 | C |
| ATOM | 24132 | CB | SER | D | 1 | −39.181 | 59.855 | −16.046 | 1.00 | 125.13 | C |
| ATOM | 24135 | OG | SER | D | 1 | −39.072 | 61.271 | −16.142 | 1.00 | 101.73 | O |
| ATOM | 24137 | C | SER | D | 1 | −37.808 | 57.787 | −15.462 | 1.00 | 124.90 | C |
| ATOM | 24138 | O | SER | D | 1 | −37.636 | 57.794 | −14.234 | 1.00 | 119.71 | O |
| ATOM | 24141 | N | ALA | D | 2 | −37.993 | 56.674 | −16.190 | 1.00 | 123.14 | N |
| ATOM | 24143 | CA | ALA | D | 2 | −37.775 | 55.282 | −15.725 | 1.00 | 120.62 | C |
| ATOM | 24145 | CB | ALA | D | 2 | −36.933 | 55.216 | −14.441 | 1.00 | 120.30 | C |
| ATOM | 24149 | C | ALA | D | 2 | −39.048 | 54.444 | −15.549 | 1.00 | 117.12 | C |
| ATOM | 24150 | O | ALA | D | 2 | −40.135 | 54.967 | −15.278 | 1.00 | 116.07 | O |
| ATOM | 24151 | N | VAL | D | 3 | −38.902 | 53.138 | −15.755 | 1.00 | 115.36 | N |
| ATOM | 24153 | CA | VAL | D | 3 | −39.526 | 52.106 | −14.920 | 1.00 | 114.04 | C |
| ATOM | 24155 | CB | VAL | D | 3 | −40.945 | 51.658 | −15.415 | 1.00 | 116.49 | C |
| ATOM | 24157 | CG1 | VAL | D | 3 | −41.544 | 50.608 | −14.466 | 1.00 | 117.12 | C |
| ATOM | 24161 | CG2 | VAL | D | 3 | −41.908 | 52.848 | −15.570 | 1.00 | 116.41 | C |
| ATOM | 24165 | C | VAL | D | 3 | −38.616 | 50.877 | −14.952 | 1.00 | 108.58 | C |
| ATOM | 24166 | O | VAL | D | 3 | −38.295 | 50.324 | −13.901 | 1.00 | 111.25 | O |
| ATOM | 24167 | N | LEU | D | 4 | −38.195 | 50.467 | −16.150 | 1.00 | 97.93 | N |
| ATOM | 24169 | CA | LEU | D | 4 | −37.490 | 49.202 | −16.331 | 1.00 | 91.70 | C |
| ATOM | 24171 | CB | LEU | D | 4 | −38.005 | 48.488 | −17.585 | 1.00 | 95.32 | C |
| ATOM | 24174 | CG | LEU | D | 4 | −38.775 | 47.198 | −17.246 | 1.00 | 111.63 | C |
| ATOM | 24176 | CD1 | LEU | D | 4 | −40.321 | 47.319 | −17.385 | 1.00 | 109.86 | C |
| ATOM | 24180 | CD2 | LEU | D | 4 | −38.205 | 46.011 | −18.052 | 1.00 | 120.59 | C |
| ATOM | 24184 | C | LEU | D | 4 | −35.988 | 49.421 | −16.442 | 1.00 | 81.26 | C |
| ATOM | 24185 | O | LEU | D | 4 | −35.575 | 50.220 | −17.288 | 1.00 | 70.10 | O |
| ATOM | 24186 | N | GLN | D | 5 | −35.209 | 48.743 | −15.581 | 1.00 | 67.72 | N |
| ATOM | 24188 | CA | GLN | D | 5 | −33.737 | 48.725 | −15.652 | 1.00 | 57.46 | C |
| ATOM | 24190 | CB | GLN | D | 5 | −33.096 | 48.019 | −14.455 | 1.00 | 49.08 | C |
| ATOM | 24193 | CG | GLN | D | 5 | −31.890 | 48.670 | −13.734 | 1.00 | 54.48 | C |
| ATOM | 24196 | CD | GLN | D | 5 | −30.988 | 49.624 | −14.519 | 1.00 | 49.57 | C |
| ATOM | 24197 | OE1 | GLN | D | 5 | −30.681 | 50.697 | −13.997 | 1.00 | 63.66 | O |
| ATOM | 24198 | NE2 | GLN | D | 5 | −30.449 | 49.201 | −15.662 | 1.00 | 33.27 | N |
| ATOM | 24201 | C | GLN | D | 5 | −33.306 | 47.938 | −16.876 | 1.00 | 50.81 | C |
| ATOM | 24202 | O | GLN | D | 5 | −33.570 | 46.749 | −16.941 | 1.00 | 43.57 | O |
| ATOM | 24203 | N | LYS | D | 6 | −32.588 | 48.586 | −17.790 | 1.00 | 45.52 | N |
| ATOM | 24205 | CA | LYS | D | 6 | −32.110 | 47.923 | −18.996 | 1.00 | 50.50 | C |
| ATOM | 24207 | CB | LYS | D | 6 | −31.910 | 48.913 | −20.152 | 1.00 | 54.38 | C |
| ATOM | 24210 | CG | LYS | D | 6 | −33.229 | 49.469 | −20.715 | 1.00 | 65.92 | C |
| ATOM | 24213 | CD | LYS | D | 6 | −34.073 | 48.415 | −21.431 | 1.00 | 70.58 | C |
| ATOM | 24216 | CE | LYS | D | 6 | −33.586 | 48.211 | −22.869 | 1.00 | 87.93 | C |
| ATOM | 24219 | NZ | LYS | D | 6 | −33.925 | 46.837 | −23.357 | 1.00 | 83.60 | N |
| ATOM | 24223 | C | LYS | D | 6 | −30.818 | 47.215 | −18.670 | 1.00 | 46.91 | C |
| ATOM | 24224 | O | LYS | D | 6 | −30.287 | 47.384 | −17.578 | 1.00 | 46.46 | O |
| ATOM | 24225 | N | LYS | D | 7 | −30.356 | 46.359 | −19.572 | 1.00 | 44.65 | N |
| ATOM | 24227 | CA | LYS | D | 7 | −29.185 | 45.564 | −19.245 | 1.00 | 48.12 | C |
| ATOM | 24229 | CB | LYS | D | 7 | −29.548 | 44.087 | −19.276 | 1.00 | 47.12 | C |
| ATOM | 24232 | CG | LYS | D | 7 | −29.932 | 43.486 | −17.932 | 1.00 | 57.71 | C |
| ATOM | 24235 | CD | LYS | D | 7 | −31.033 | 42.457 | −18.127 | 1.00 | 68.92 | C |
| ATOM | 24238 | CE | LYS | D | 7 | −30.646 | 41.072 | −17.644 | 1.00 | 93.96 | C |
| ATOM | 24241 | NZ | LYS | D | 7 | −31.280 | 40.786 | −16.312 | 1.00 | 107.71 | N |
| ATOM | 24245 | C | LYS | D | 7 | −28.034 | 45.847 | −20.210 | 1.00 | 51.45 | C |
| ATOM | 24246 | O | LYS | D | 7 | −20.263 | 46.141 | −21.392 | 1.00 | 56.67 | O |
| ATOM | 24247 | N | ILE | D | 8 | −26.799 | 45.722 | −19.732 | 1.00 | 44.47 | N |
| ATOM | 24249 | CA | ILE | D | 8 | −25.659 | 45.942 | −20.615 | 1.00 | 48.02 | C |
| ATOM | 24251 | CB | ILE | D | 8 | −24.331 | 45.704 | −19.867 | 1.00 | 50.38 | C |
| ATOM | 24253 | CG1 | ILE | D | 8 | −24.027 | 46.888 | −18.931 | 1.00 | 47.92 | C |
| ATOM | 24256 | CD1 | ILE | D | 8 | −23.488 | 48.121 | −19.618 | 1.00 | 57.34 | C |
| ATOM | 24260 | CG2 | ILE | D | 8 | −23.194 | 45.454 | −20.861 | 1.00 | 55.24 | C |
| ATOM | 24264 | C | ILE | D | 8 | −25.757 | 45.074 | −21.872 | 1.00 | 50.58 | C |
| ATOM | 24265 | O | ILE | D | 8 | −25.279 | 45.444 | −22.954 | 1.00 | 52.23 | O |
| ATOM | 24266 | N | THR | D | 9 | −26.415 | 43.925 | −21.739 | 1.00 | 52.37 | N |
| ATOM | 24268 | CA | THR | D | 9 | −26.458 | 42.928 | −22.805 | 1.00 | 44.74 | C |
| ATOM | 24270 | CB | THR | D | 9 | −26.820 | 41.591 | −22.223 | 1.00 | 40.53 | C |
| ATOM | 24272 | OG1 | THR | D | 9 | −27.873 | 41.763 | −21.270 | 1.00 | 56.53 | O |
| ATOM | 24274 | CG2 | THR | D | 9 | −25.711 | 41.077 | −21.425 | 1.00 | 37.86 | C |
| ATOM | 24278 | C | THR | D | 9 | −27.467 | 43.266 | −23.880 | 1.00 | 37.90 | C |
| ATOM | 24279 | O | THR | D | 9 | −27.487 | 42.616 | −24.927 | 1.00 | 35.58 | O |
| ATOM | 24280 | N | ASP | D | 10 | −28.295 | 44.270 | −23.603 | 1.00 | 38.65 | N |
| ATOM | 24282 | CA | ASP | D | 10 | −29.245 | 44.803 | −24.572 | 1.00 | 32.47 | C |
| ATOM | 24284 | CB | ASP | D | 10 | −30.361 | 45.538 | −23.849 | 1.00 | 35.09 | C |
| ATOM | 24287 | CG | ASP | D | 10 | −31.146 | 44.677 | −22.881 | 1.00 | 43.86 | C |
| ATOM | 24288 | OD1 | ASP | D | 10 | −30.981 | 43.435 | −22.845 | 1.00 | 57.96 | O |
| ATOM | 24289 | OD2 | ASP | D | 10 | −31.906 | 45.218 | −22.048 | 1.00 | 47.08 | O |
| ATOM | 24290 | C | ASP | D | 10 | −28.587 | 45.824 | −25.479 | 1.00 | 41.21 | C |
| ATOM | 24291 | O | ASP | D | 10 | −29.258 | 46.473 | −26.285 | 1.00 | 57.46 | O |
| ATOM | 24292 | N | TYR | D | 11 | −27.287 | 46.040 | −25.319 | 1.00 | 46.26 | N |
| ATOM | 24294 | CA | TYR | D | 11 | −26.590 | 47.018 | −26.142 | 1.00 | 40.85 | C |
| ATOM | 24296 | CB | TYR | D | 11 | −26.329 | 48.295 | −25.357 | 1.00 | 37.24 | C |
| ATOM | 24299 | CG | TYR | D | 11 | −27.530 | 48.880 | −24.639 | 1.00 | 40.31 | C |
| ATOM | 24300 | CD1 | TYR | D | 11 | −27.823 | 48.561 | −23.323 | 1.00 | 42.00 | C |
| ATOM | 24302 | CE1 | TYR | D | 11 | −28.890 | 49.168 | −22.654 | 1.00 | 47.16 | C |
| ATOM | 24304 | CZ | TYR | D | 11 | −29.601 | 50.146 | −23.276 | 1.00 | 33.98 | C |
| ATOM | 24305 | OH | TYR | D | 11 | −30.705 | 50.703 | −22.700 | 1.00 | 63.26 | O |
| ATOM | 24307 | CE2 | TYR | D | 11 | −29.327 | 50.471 | −24.570 | 1.00 | 37.42 | C |
| ATOM | 24309 | CD2 | TYR | D | 11 | −28.318 | 49.838 | −25.248 | 1.00 | 46.76 | C |
| ATOM | 24311 | C | TYR | D | 11 | −25.272 | 46.447 | −26.606 | 1.00 | 43.39 | C |
| ATOM | 24312 | O | TYR | D | 11 | −24.855 | 46.747 | −27.707 | 1.00 | 50.09 | O |
| ATOM | 24313 | N | PHE | D | 12 | −24.584 | 45.684 | −25.757 | 1.00 | 48.13 | N |
| ATOM | 24315 | CA | PHE | D | 12 | −23.280 | 45.111 | −26.110 | 1.00 | 46.49 | C |
| ATOM | 24317 | CB | PHE | D | 12 | −22.269 | 45.408 | −25.018 | 1.00 | 45.61 | C |
| ATOM | 24320 | CG | PHE | D | 12 | −22.106 | 46.867 | −24.726 | 1.00 | 48.19 | C |
| ATOM | 24321 | CD1 | PHE | D | 12 | −22.533 | 47.826 | −25.634 | 1.00 | 45.10 | C |
| ATOM | 24323 | CE1 | PHE | D | 12 | −22.417 | 49.154 | −25.338 | 1.00 | 40.17 | C |
| ATOM | 24325 | CZ | PHE | D | 12 | −21.833 | 49.534 | −24.132 | 1.00 | 46.68 | C |
| ATOM | 24327 | CE2 | PHE | D | 12 | −21.410 | 48.596 | −23.229 | 1.00 | 45.44 | C |
| ATOM | 24329 | CD2 | PHE | D | 12 | −21.538 | 47.271 | −23.530 | 1.00 | 47.56 | C |
| ATOM | 24331 | C | PHE | D | 12 | −23.361 | 43.598 | −26.270 | 1.00 | 49.08 | C |
| ATOM | 24332 | O | PHE | D | 12 | −23.939 | 42.888 | −25.448 | 1.00 | 50.78 | O |
| ATOM | 24333 | N | HIS | D | 13 | −22.781 | 43.086 | −27.341 | 1.00 | 48.57 | N |
| ATOM | 24335 | CA | HIS | D | 13 | −23.087 | 41.724 | −27.745 | 1.00 | 43.11 | C |
| ATOM | 24337 | CB | HIS | D | 13 | −23.868 | 41.731 | −29.066 | 1.00 | 49.67 | C |
| ATOM | 24340 | CG | HIS | D | 13 | −25.135 | 42.542 | −29.036 | 1.00 | 60.35 | C |
| ATOM | 24341 | ND1 | HIS | D | 13 | −25.579 | 43.277 | −30.120 | 1.00 | 80.63 | N |
| ATOM | 24343 | CE1 | HIS | D | 13 | −26.715 | 43.884 | −29.817 | 1.00 | 65.52 | C |
| ATOM | 24345 | NE2 | HIS | D | 13 | −27.029 | 43.563 | −28.575 | 1.00 | 71.84 | N |
| ATOM | 24347 | CD2 | HIS | D | 13 | −26.076 | 42.702 | −28.074 | 1.00 | 61.78 | C |
| ATOM | 24349 | C | HIS | D | 13 | −21.750 | 41.026 | −27.899 | 1.00 | 37.33 | C |
| ATOM | 24350 | O | HIS | D | 13 | −20.726 | 41.676 | −28.058 | 1.00 | 43.88 | O |
| ATOM | 24351 | N | PRO | D | 14 | −21.723 | 39.711 | −27.764 | 1.00 | 43.69 | N |
| ATOM | 24352 | CA | PRO | D | 14 | −20.471 | 38.965 | −27.838 | 1.00 | 47.92 | C |
| ATOM | 24354 | CB | PRO | D | 14 | −20.927 | 37.516 | −27.780 | 1.00 | 50.84 | C |
| ATOM | 24357 | CG | PRO | D | 14 | −22.180 | 37.562 | −27.014 | 1.00 | 48.30 | C |
| ATOM | 24360 | CD | PRO | D | 14 | −22.852 | 38.836 | −27.417 | 1.00 | 50.06 | C |
| ATOM | 24363 | C | PRO | D | 14 | −19.792 | 39.199 | −29.147 | 1.00 | 57.56 | C |
| ATOM | 24364 | O | PRO | D | 14 | −20.496 | 39.238 | −30.159 | 1.00 | 53.68 | O |
| ATOM | 24365 | N | LYS | D | 15 | −18.462 | 39.244 | −29.095 | 1.00 | 71.14 | N |
| ATOM | 24367 | CA | LYS | D | 15 | −17.608 | 39.803 | −30.143 | 1.00 | 80.01 | C |
| ATOM | 24369 | CB | LYS | D | 15 | −16.301 | 40.288 | −29.501 | 1.00 | 77.59 | C |
| ATOM | 24372 | CG | LYS | D | 15 | −15.621 | 41.483 | −30.167 | 1.00 | 89.77 | C |
| ATOM | 24375 | CD | LYS | D | 15 | −14.665 | 42.214 | −29.195 | 1.00 | 103.18 | C |
| ATOM | 24378 | CE | LYS | D | 15 | −13.368 | 42.711 | −29.841 | 1.00 | 97.29 | C |
| ATOM | 24381 | NZ | LYS | D | 15 | −12.200 | 42.460 | −28.945 | 1.00 | 104.64 | N |
| ATOM | 24385 | C | LYS | D | 15 | −17.317 | 38.783 | −31.267 | 1.00 | 87.43 | C |
| ATOM | 24386 | O | LYS | D | 15 | −16.750 | 39.145 | −32.306 | 1.00 | 86.52 | O |
| ATOM | 24387 | N | LYS | D | 16 | −17.708 | 37.522 | −31.059 | 1.00 | 93.73 | N |
| ATOM | 24389 | CA | LYS | D | 16 | −18.030 | 36.603 | −32.161 | 1.00 | 99.07 | C |
| ATOM | 24391 | CB | LYS | D | 16 | −16.752 | 36.159 | −32.907 | 1.00 | 100.24 | C |
| ATOM | 24394 | CG | LYS | D | 16 | −16.801 | 36.320 | −34.436 | 1.00 | 102.96 | C |
| ATOM | 24397 | CD | LYS | D | 16 | −16.822 | 37.789 | −34.900 | 1.00 | 107.07 | C |
| ATOM | 24400 | CE | LYS | D | 16 | −18.205 | 38.457 | −34.789 | 1.00 | 98.81 | C |
| ATOM | 24403 | NZ | LYS | D | 16 | −18.200 | 39.864 | −35.304 | 1.00 | 92.27 | N |
| ATOM | 24407 | C | LYS | D | 16 | −18.835 | 35.385 | −31.666 | 1.00 | 99.17 | C |
| ATOM | 24408 | O | LYS | D | 16 | −18.746 | 35.065 | −30.477 | 1.00 | 96.34 | O |
| ATOM | 24409 | OXT | LYS | D | 16 | −19.598 | 34.733 | −32.408 | 1.00 | 94.39 | O |
| ATOM | 24410 | N | SER | F | 1 | 3.132 | 21.154 | 5.361 | 1.00 | 121.01 | N |
| ATOM | 24412 | CA | SER | F | 1 | 3.255 | 19.824 | 6.025 | 1.00 | 121.01 | C |
| ATOM | 24414 | CB | SER | F | 1 | 4.727 | 19.458 | 6.272 | 1.00 | 121.42 | C |
| ATOM | 24417 | OG | SER | F | 1 | 5.581 | 20.587 | 6.209 | 1.00 | 117.96 | O |
| ATOM | 24419 | C | SER | F | 1 | 2.421 | 19.741 | 7.317 | 1.00 | 120.21 | C |
| ATOM | 24420 | O | SER | F | 1 | 1.203 | 19.569 | 7.239 | 1.00 | 122.09 | O |
| ATOM | 24423 | N | ALA | F | 2 | 3.054 | 19.830 | 8.489 | 1.00 | 116.11 | N |
| ATOM | 24425 | CA | ALA | F | 2 | 2.587 | 19.100 | 9.673 | 1.00 | 111.82 | C |
| ATOM | 24427 | CB | ALA | F | 2 | 3.452 | 17.850 | 9.917 | 1.00 | 111.69 | C |
| ATOM | 24431 | C | ALA | F | 2 | 2.548 | 19.983 | 10.926 | 1.00 | 106.83 | C |
| ATOM | 24432 | O | ALA | F | 2 | 3.407 | 20.851 | 11.095 | 1.00 | 100.06 | O |
| ATOM | 24433 | N | VAL | F | 3 | 1.546 | 19.770 | 11.786 | 1.00 | 101.74 | N |
| ATOM | 24435 | CA | VAL | F | 3 | 1.321 | 20.654 | 12.923 | 1.00 | 91.99 | C |
| ATOM | 24437 | CB | VAL | F | 3 | 1.465 | 22.152 | 12.512 | 1.00 | 93.47 | C |
| ATOM | 24439 | CG1 | VAL | F | 3 | 2.946 | 22.526 | 12.288 | 1.00 | 84.26 | C |
| ATOM | 24443 | CG2 | VAL | F | 3 | 0.571 | 22.528 | 11.308 | 1.00 | 90.03 | C |
| ATOM | 24447 | C | VAL | F | 3 | 0.043 | 20.429 | 13.759 | 1.00 | 87.88 | C |
| ATOM | 24448 | O | VAL | F | 3 | −1.091 | 20.512 | 13.282 | 1.00 | 81.64 | O |
| ATOM | 24449 | N | LEU | F | 4 | 0.260 | 20.214 | 15.054 | 1.00 | 83.89 | N |
| ATOM | 24451 | CA | LEU | F | 4 | −0.755 | 20.429 | 16.080 | 1.00 | 77.21 | C |
| ATOM | 24453 | CB | LEU | F | 4 | −1.202 | 19.078 | 16.622 | 1.00 | 88.66 | C |
| ATOM | 24456 | CG | LEU | F | 4 | −0.041 | 18.116 | 16.905 | 1.00 | 95.39 | C |
| ATOM | 24458 | CD1 | LEU | F | 4 | −0.095 | 17.647 | 18.378 | 1.00 | 99.81 | C |
| ATOM | 24462 | CD2 | LEU | F | 4 | −0.092 | 16.938 | 15.882 | 1.00 | 100.52 | C |
| ATOM | 24466 | C | LEU | F | 4 | −0.195 | 21.190 | 17.277 | 1.00 | 67.38 | C |
| ATOM | 24467 | O | LEU | F | 4 | 1.006 | 21.124 | 17.551 | 1.00 | 49.55 | O |
| ATOM | 24468 | N | GLN | F | 5 | −1.112 | 21.798 | 18.034 | 1.00 | 55.08 | N |
| ATOM | 24470 | CA | GLN | F | 5 | −0.835 | 22.948 | 18.873 | 1.00 | 45.15 | C |
| ATOM | 24472 | CB | GLN | F | 5 | −2.091 | 23.804 | 19.028 | 1.00 | 48.37 | C |
| ATOM | 24475 | CG | GLN | F | 5 | −1.953 | 24.995 | 19.987 | 1.00 | 58.05 | C |
| ATOM | 24478 | CD | GLN | F | 5 | −0.908 | 25.983 | 19.523 | 1.00 | 56.55 | C |
| ATOM | 24479 | OE1 | GLN | F | 5 | −1.074 | 26.644 | 18.484 | 1.00 | 59.48 | O |
| ATOM | 24480 | NE2 | GLN | F | 5 | 0.204 | 26.034 | 20.249 | 1.00 | 48.94 | N |
| ATOM | 24483 | C | GLN | F | 5 | −0.470 | 22.443 | 20.235 | 1.00 | 49.13 | C |
| ATOM | 24484 | O | GLN | F | 5 | −1.329 | 21.935 | 20.950 | 1.00 | 58.28 | O |
| ATOM | 24485 | N | LYS | F | 6 | 0.763 | 22.678 | 20.652 | 1.00 | 47.99 | N |
| ATOM | 24487 | CA | LYS | F | 6 | 1.123 | 22.423 | 22.035 | 1.00 | 49.23 | C |
| ATOM | 24489 | CB | LYS | F | 6 | 2.647 | 22.301 | 22.216 | 1.00 | 57.89 | C |
| ATOM | 24492 | CG | LYS | F | 6 | 3.313 | 21.133 | 21.493 | 1.00 | 57.52 | C |
| ATOM | 24495 | CD | LYS | F | 6 | 2.616 | 19.835 | 21.845 | 1.00 | 80.62 | C |
| ATOM | 24498 | CE | LYS | F | 6 | 2.995 | 19.361 | 23.241 | 1.00 | 93.05 | C |
| ATOM | 24501 | NZ | LYS | F | 6 | 2.861 | 17.878 | 23.387 | 1.00 | 97.95 | N |
| ATOM | 24505 | C | LYS | F | 6 | 0.569 | 23.530 | 22.923 | 1.00 | 45.41 | C |
| ATOM | 24506 | O | LYS | F | 6 | −0.095 | 24.457 | 22.468 | 1.00 | 51.38 | O |
| ATOM | 24507 | N | LYS | F | 7 | 0.808 | 23.365 | 24.215 | 1.00 | 50.84 | N |
| ATOM | 24509 | CA | LYS | F | 7 | 0.140 | 24.121 | 25.258 | 1.00 | 52.85 | C |
| ATOM | 24511 | CB | LYS | F | 7 | −0.914 | 23.251 | 25.954 | 1.00 | 54.28 | C |
| ATOM | 24514 | CG | LYS | F | 7 | −2.361 | 23.670 | 25.682 | 1.00 | 57.65 | C |
| ATOM | 24517 | CD | LYS | F | 7 | −3.421 | 22.535 | 25.706 | 1.00 | 61.91 | C |
| ATOM | 24520 | CE | LYS | F | 7 | −4.611 | 22.811 | 24.740 | 1.00 | 80.41 | C |
| ATOM | 24523 | NZ | LYS | F | 7 | −5.879 | 23.374 | 25.329 | 1.00 | 73.72 | N |
| ATOM | 24527 | C | LYS | F | 7 | 1.242 | 24.559 | 26.232 | 1.00 | 56.48 | C |
| ATOM | 24528 | O | LYS | F | 7 | 2.287 | 23.904 | 26.375 | 1.00 | 53.27 | O |
| ATOM | 24529 | N | ILE | F | 8 | 1.041 | 25.719 | 26.841 | 1.00 | 55.93 | N |
| ATOM | 24531 | CA | ILE | F | 8 | 2.089 | 26.317 | 27.639 | 1.00 | 52.64 | C |
| ATOM | 24533 | CB | ILE | F | 8 | 1.618 | 27.660 | 28.191 | 1.00 | 51.74 | C |
| ATOM | 24535 | CG1 | ILE | F | 8 | 1.768 | 28.740 | 27.122 | 1.00 | 62.17 | C |
| ATOM | 24538 | CD1 | ILE | F | 8 | 3.221 | 29.162 | 26.828 | 1.00 | 57.51 | C |
| ATOM | 24542 | CG2 | ILE | F | 8 | 2.431 | 28.036 | 29.409 | 1.00 | 58.88 | C |
| ATOM | 24546 | C | ILE | F | 8 | 2.422 | 25.346 | 28.758 | 1.00 | 52.38 | C |
| ATOM | 24547 | O | ILE | F | 8 | 3.571 | 25.227 | 29.151 | 1.00 | 52.77 | O |
| ATOM | 24548 | N | THR | F | 9 | 1.421 | 24.612 | 29.233 | 1.00 | 52.73 | N |
| ATOM | 24550 | CA | THR | F | 9 | 1.594 | 23.668 | 30.335 | 1.00 | 53.63 | C |
| ATOM | 24552 | CB | THR | F | 9 | 0.226 | 23.151 | 30.746 | 1.00 | 55.80 | C |
| ATOM | 24554 | OG1 | THR | F | 9 | −0.346 | 22.409 | 29.660 | 1.00 | 84.98 | O |
| ATOM | 24556 | CG2 | THR | F | 9 | −0.770 | 24.276 | 30.918 | 1.00 | 52.45 | C |
| ATOM | 24560 | C | THR | F | 9 | 2.479 | 22.467 | 29.967 | 1.00 | 49.05 | C |
| ATOM | 24561 | O | THR | F | 9 | 2.960 | 21.743 | 30.811 | 1.00 | 57.13 | O |
| ATOM | 24562 | N | ASP | F | 10 | 2.766 | 22.288 | 28.694 | 1.00 | 50.79 | N |
| ATOM | 24564 | CA | ASP | F | 10 | 3.740 | 21.297 | 28.269 | 1.00 | 50.05 | C |
| ATOM | 24566 | CB | ASP | F | 10 | 3.470 | 20.862 | 26.797 | 1.00 | 48.78 | C |
| ATOM | 24569 | CG | ASP | F | 10 | 2.021 | 20.331 | 26.576 | 1.00 | 62.38 | C |
| ATOM | 24570 | OD1 | ASP | F | 10 | 1.388 | 19.819 | 27.538 | 1.00 | 48.91 | O |
| ATOM | 24571 | OD2 | ASP | F | 10 | 1.394 | 20.425 | 25.493 | 1.00 | 51.70 | O |
| ATOM | 24572 | C | ASP | F | 10 | 5.179 | 21.765 | 28.468 | 1.00 | 44.75 | C |
| ATOM | 24573 | O | ASP | F | 10 | 6.088 | 21.076 | 28.056 | 1.00 | 54.54 | O |
| ATOM | 24574 | N | TYR | F | 11 | 5.429 | 22.951 | 29.006 | 1.00 | 51.25 | N |
| ATOM | 24576 | CA | TYR | F | 11 | 6.797 | 23.512 | 28.954 | 1.00 | 50.51 | C |
| ATOM | 24578 | CB | TYR | F | 11 | 7.017 | 24.562 | 27.841 | 1.00 | 42.59 | C |
| ATOM | 24581 | CG | TYR | F | 11 | 6.767 | 24.084 | 26.422 | 1.00 | 46.94 | C |
| ATOM | 24582 | CD1 | TYR | F | 11 | 5.502 | 24.171 | 25.847 | 1.00 | 54.08 | C |
| ATOM | 24584 | CE1 | TYR | F | 11 | 5.261 | 23.692 | 24.582 | 1.00 | 43.86 | C |
| ATOM | 24586 | CZ | TYR | F | 11 | 6.305 | 23.163 | 23.848 | 1.00 | 57.41 | C |
| ATOM | 24587 | OH | TYR | F | 11 | 6.107 | 22.692 | 22.568 | 1.00 | 59.06 | O |
| ATOM | 24589 | CE2 | TYR | F | 11 | 7.562 | 23.089 | 24.384 | 1.00 | 45.56 | C |
| ATOM | 24591 | CD2 | TYR | F | 11 | 7.784 | 23.547 | 25.658 | 1.00 | 43.38 | C |
| ATOM | 24593 | C | TYR | F | 11 | 7.109 | 24.164 | 30.297 | 1.00 | 53.84 | C |
| ATOM | 24594 | O | TYR | F | 11 | 8.234 | 24.085 | 30.772 | 1.00 | 54.86 | O |
| ATOM | 24595 | N | PHE | F | 12 | 6.129 | 24.852 | 30.869 | 1.00 | 53.67 | N |
| ATOM | 24597 | CA | PHE | F | 12 | 6.315 | 25.535 | 32.134 | 1.00 | 57.22 | C |
| ATOM | 24599 | CB | PHE | F | 12 | 5.927 | 27.012 | 32.045 | 1.00 | 56.23 | C |
| ATOM | 24602 | CG | PHE | F | 12 | 6.680 | 27.783 | 31.025 | 1.00 | 53.37 | C |
| ATOM | 24603 | CD1 | PHE | F | 12 | 7.968 | 27.426 | 30.672 | 1.00 | 53.18 | C |
| ATOM | 24605 | CE1 | PHE | F | 12 | 8.664 | 28.145 | 29.689 | 1.00 | 57.92 | C |
| ATOM | 24607 | CZ | PHE | F | 12 | 8.074 | 29.260 | 29.085 | 1.00 | 45.49 | C |
| ATOM | 24609 | CE2 | PHE | F | 12 | 6.796 | 29.638 | 29.464 | 1.00 | 35.76 | C |
| ATOM | 24611 | CD2 | PHE | F | 12 | 6.094 | 28.886 | 30.414 | 1.00 | 51.37 | C |
| ATOM | 24613 | C | PHE | F | 12 | 5.369 | 24.877 | 33.113 | 1.00 | 62.91 | C |
| ATOM | 24614 | O | PHE | F | 12 | 4.163 | 24.802 | 32.877 | 1.00 | 62.83 | O |
| ATOM | 24615 | N | HIS | F | 13 | 5.910 | 24.463 | 34.251 | 1.00 | 67.97 | N |
| ATOM | 24617 | CA | HIS | F | 13 | 5.129 | 23.693 | 35.214 | 1.00 | 65.62 | C |
| ATOM | 24619 | CB | HIS | F | 13 | 5.884 | 22.390 | 35.499 | 1.00 | 65.73 | C |
| ATOM | 24622 | CG | HIS | F | 13 | 6.074 | 21.541 | 34.279 | 1.00 | 80.29 | C |
| ATOM | 24623 | ND1 | HIS | F | 13 | 5.776 | 21.994 | 33.010 | 1.00 | 100.73 | N |
| ATOM | 24625 | CE1 | HIS | F | 13 | 6.004 | 21.033 | 32.131 | 1.00 | 100.67 | C |
| ATOM | 24627 | NE2 | HIS | F | 13 | 6.431 | 19.968 | 32.786 | 1.00 | 108.85 | N |
| ATOM | 24629 | CD2 | HIS | F | 13 | 6.470 | 20.255 | 34.132 | 1.00 | 98.09 | C |
| ATOM | 24631 | C | HIS | F | 13 | 4.974 | 24.616 | 36.417 | 1.00 | 62.02 | C |
| ATOM | 24632 | O | HIS | F | 13 | 5.685 | 25.615 | 36.490 | 1.00 | 58.40 | O |
| ATOM | 24633 | N | PRO | F | 14 | 3.997 | 24.377 | 37.289 | 1.00 | 65.26 | N |
| ATOM | 24634 | CA | PRO | F | 14 | 3.825 | 25.209 | 38.495 | 1.00 | 68.16 | C |
| ATOM | 24636 | CB | PRO | F | 14 | 2.777 | 24.446 | 39.323 | 1.00 | 57.65 | C |
| ATOM | 24639 | CG | PRO | F | 14 | 2.002 | 23.678 | 38.338 | 1.00 | 58.74 | C |
| ATOM | 24642 | CD | PRO | F | 14 | 2.955 | 23.341 | 37.189 | 1.00 | 67.06 | C |
| ATOM | 24645 | C | PRO | F | 14 | 5.126 | 25.327 | 39.271 | 1.00 | 68.37 | C |
| ATOM | 24646 | O | PRO | F | 14 | 5.738 | 24.279 | 39.425 | 1.00 | 65.55 | O |
| ATOM | 24647 | N | LYS | F | 15 | 5.538 | 26.529 | 39.676 | 1.00 | 73.29 | N |
| ATOM | 24649 | CA | LYS | F | 15 | 6.433 | 26.749 | 40.815 | 1.00 | 83.18 | C |
| ATOM | 24651 | CB | LYS | F | 15 | 6.446 | 28.244 | 41.167 | 1.00 | 84.60 | C |
| ATOM | 24654 | CG | LYS | F | 15 | 7.621 | 29.080 | 40.671 | 1.00 | 77.68 | C |
| ATOM | 24657 | CD | LYS | F | 15 | 7.154 | 30.522 | 40.419 | 1.00 | 77.21 | C |
| ATOM | 24660 | CE | LYS | F | 15 | 7.455 | 31.444 | 41.588 | 1.00 | 79.26 | C |
| ATOM | 24663 | NZ | LYS | F | 15 | 8.257 | 32.625 | 41.170 | 1.00 | 93.16 | N |
| ATOM | 24667 | C | LYS | F | 15 | 5.923 | 26.030 | 42.067 | 1.00 | 96.88 | C |
| ATOM | 24668 | O | LYS | F | 15 | 5.352 | 26.665 | 42.946 | 1.00 | 95.51 | O |
| ATOM | 24669 | N | LYS | F | 16 | 6.117 | 24.718 | 42.174 | 1.00 | 114.48 | N |
| ATOM | 24671 | CA | LYS | F | 16 | 5.765 | 23.998 | 43.402 | 1.00 | 122.65 | C |
| ATOM | 24673 | CB | LYS | F | 16 | 5.485 | 22.500 | 43.144 | 1.00 | 123.57 | C |
| ATOM | 24676 | CG | LYS | F | 16 | 6.326 | 21.840 | 42.038 | 1.00 | 126.33 | C |
| ATOM | 24679 | CD | LYS | F | 16 | 7.410 | 20.889 | 42.565 | 1.00 | 125.34 | C |
| ATOM | 24682 | CE | LYS | F | 16 | 7.767 | 19.803 | 41.532 | 1.00 | 123.57 | C |
| ATOM | 24685 | NZ | LYS | F | 16 | 7.123 | 18.485 | 41.843 | 1.00 | 118.30 | N |
| ATOM | 24689 | C | LYS | F | 16 | 6.899 | 24.194 | 44.415 | 1.00 | 127.29 | C |
| ATOM | 24690 | O | LYS | F | 16 | 7.754 | 25.068 | 44.214 | 1.00 | 125.26 | O |
| ATOM | 24691 | OXT | LYS | F | 16 | 6.966 | 23.489 | 45.430 | 1.00 | 133.29 | O |
| ATOM | 24692 | N | SER | H | 1 | −16.990 | 56.875 | 105.633 | 1.00 | 129.41 | N |
| ATOM | 24694 | CA | SER | H | 1 | −15.809 | 56.588 | 106.505 | 1.00 | 127.97 | C |
| ATOM | 24696 | CB | SER | H | 1 | −15.846 | 57.488 | 107.741 | 1.00 | 125.91 | C |
| ATOM | 24699 | OG | SER | H | 1 | −16.541 | 58.685 | 107.437 | 1.00 | 124.74 | O |
| ATOM | 24701 | C | SER | H | 1 | −15.765 | 55.114 | 106.912 | 1.00 | 127.36 | C |
| ATOM | 24702 | O | SER | H | 1 | −16.756 | 54.392 | 106.793 | 1.00 | 132.30 | O |
| ATOM | 24705 | N | ALA | H | 2 | −14.603 | 54.662 | 107.370 | 1.00 | 122.93 | N |
| ATOM | 24707 | CA | ALA | H | 2 | −14.341 | 53.237 | 107.538 | 1.00 | 115.87 | C |
| ATOM | 24709 | CB | ALA | H | 2 | −15.003 | 52.430 | 106.432 | 1.00 | 113.00 | C |
| ATOM | 24713 | C | ALA | H | 2 | −12.835 | 53.010 | 107.548 | 1.00 | 109.99 | C |
| ATOM | 24714 | O | ALA | H | 2 | −12.209 | 52.890 | 106.503 | 1.00 | 106.03 | O |
| ATOM | 24715 | N | VAL | H | 3 | −12.251 | 52.996 | 108.739 | 1.00 | 109.50 | N |
| ATOM | 24717 | CA | VAL | H | 3 | −10.805 | 52.849 | 108.873 | 1.00 | 106.54 | C |
| ATOM | 24719 | CB | VAL | H | 3 | −10.311 | 53.073 | 110.343 | 1.00 | 108.98 | C |
| ATOM | 24721 | CG1 | VAL | H | 3 | −8.933 | 53.752 | 110.355 | 1.00 | 109.68 | C |
| ATOM | 24725 | CG2 | VAL | H | 3 | −11.323 | 53.890 | 111.174 | 1.00 | 103.79 | C |
| ATOM | 24729 | C | VAL | H | 3 | −10.357 | 51.488 | 108.302 | 1.00 | 99.24 | C |
| ATOM | 24730 | O | VAL | H | 3 | −9.618 | 51.462 | 107.322 | 1.00 | 100.32 | O |
| ATOM | 24731 | N | LEU | H | 4 | −10.830 | 50.368 | 108.852 | 1.00 | 90.05 | N |
| ATOM | 24733 | CA | LEU | H | 4 | −10.066 | 49.109 | 108.805 | 1.00 | 86.37 | C |
| ATOM | 24735 | CB | LEU | H | 4 | −10.007 | 48.429 | 110.187 | 1.00 | 89.93 | C |
| ATOM | 24738 | CG | LEU | H | 4 | −11.259 | 48.110 | 111.021 | 1.00 | 102.00 | C |
| ATOM | 24740 | CD1 | LEU | H | 4 | −12.514 | 47.920 | 110.168 | 1.00 | 106.67 | C |
| ATOM | 24744 | CD2 | LEU | H | 4 | −11.021 | 46.878 | 111.917 | 1.00 | 107.78 | C |
| ATOM | 24748 | C | LEU | H | 4 | −10.393 | 48.071 | 107.703 | 1.00 | 72.72 | C |
| ATOM | 24749 | O | LEU | H | 4 | −11.515 | 47.568 | 107.580 | 1.00 | 62.77 | O |
| ATOM | 24750 | N | GLN | H | 5 | −9.383 | 47.744 | 106.908 | 1.00 | 57.58 | N |
| ATOM | 24752 | CA | GLN | H | 5 | −9.490 | 46.621 | 106.004 | 1.00 | 60.05 | C |
| ATOM | 24754 | CB | GLN | H | 5 | −8.269 | 46.490 | 105.091 | 1.00 | 59.61 | C |
| ATOM | 24757 | CG | GLN | H | 5 | −8.454 | 45.502 | 103.927 | 1.00 | 52.10 | C |
| ATOM | 24760 | CD | GLN | H | 5 | −9.794 | 45.639 | 103.229 | 1.00 | 66.47 | C |
| ATOM | 24761 | OE1 | GLN | H | 5 | −9.919 | 46.406 | 102.266 | 1.00 | 65.48 | O |
| ATOM | 24762 | NE2 | GLN | H | 5 | −10.803 | 44.902 | 103.716 | 1.00 | 54.51 | N |
| ATOM | 24765 | C | GLN | H | 5 | −9.617 | 45.365 | 106.840 | 1.00 | 62.39 | C |
| ATOM | 24766 | O | GLN | H | 5 | −8.675 | 44.963 | 107.520 | 1.00 | 60.03 | O |
| ATOM | 24767 | N | LYS | H | 6 | −10.769 | 44.709 | 106.753 | 1.00 | 59.73 | N |
| ATOM | 24769 | CA | LYS | H | 6 | −10.843 | 43.411 | 107.367 | 1.00 | 55.40 | C |
| ATOM | 24771 | CB | LYS | H | 6 | −12.281 | 42.991 | 107.642 | 1.00 | 53.94 | C |
| ATOM | 24774 | CG | LYS | H | 6 | −12.808 | 43.569 | 108.932 | 1.00 | 63.19 | C |
| ATOM | 24777 | CD | LYS | H | 6 | −12.249 | 42.788 | 110.105 | 1.00 | 77.32 | C |
| ATOM | 24780 | CE | LYS | H | 6 | −13.080 | 42.966 | 111.365 | 1.00 | 83.24 | C |
| ATOM | 24783 | NZ | LYS | H | 6 | −13.411 | 41.666 | 112.021 | 1.00 | 80.06 | N |
| ATOM | 24787 | C | LYS | H | 6 | −10.101 | 42.464 | 106.455 | 1.00 | 48.55 | C |
| ATOM | 24788 | O | LYS | H | 6 | −9.731 | 42.817 | 105.367 | 1.00 | 48.36 | O |
| ATOM | 24789 | N | LYS | H | 7 | −9.892 | 41.256 | 106.939 | 1.00 | 56.61 | N |
| ATOM | 24791 | CA | LYS | H | 7 | −9.340 | 40.155 | 106.179 | 1.00 | 54.27 | C |
| ATOM | 24793 | CB | LYS | H | 7 | −8.170 | 39.621 | 107.000 | 1.00 | 47.23 | C |
| ATOM | 24796 | CG | LYS | H | 7 | −6.931 | 40.399 | 106.679 | 1.00 | 51.46 | C |
| ATOM | 24799 | CD | LYS | H | 7 | −6.078 | 40.650 | 107.868 | 1.00 | 52.19 | C |
| ATOM | 24802 | CE | LYS | H | 7 | −5.114 | 39.537 | 107.970 | 1.00 | 47.37 | C |
| ATOM | 24805 | NZ | LYS | H | 7 | −4.505 | 39.738 | 109.290 | 1.00 | 67.73 | N |
| ATOM | 24809 | C | LYS | H | 7 | −10.375 | 39.049 | 105.899 | 1.00 | 54.49 | C |
| ATOM | 24810 | O | LYS | H | 7 | −11.339 | 38.855 | 106.637 | 1.00 | 57.39 | O |
| ATOM | 24811 | N | ILE | H | 8 | −10.187 | 38.344 | 104.793 | 1.00 | 56.13 | N |
| ATOM | 24813 | CA | ILE | H | 8 | −11.044 | 37.223 | 104.415 | 1.00 | 50.77 | C |
| ATOM | 24815 | CB | ILE | H | 8 | −10.517 | 36.613 | 103.082 | 1.00 | 50.49 | C |
| ATOM | 24817 | CG1 | ILE | H | 8 | −10.827 | 37.516 | 101.889 | 1.00 | 50.59 | C |
| ATOM | 24820 | CD1 | ILE | H | 8 | −12.280 | 37.640 | 101.579 | 1.00 | 55.72 | C |
| ATOM | 24824 | CG2 | ILE | H | 8 | −11.062 | 35.237 | 102.835 | 1.00 | 56.75 | C |
| ATOM | 24828 | C | ILE | H | 8 | −10.991 | 36.214 | 105.568 | 1.00 | 54.11 | C |
| ATOM | 24829 | O | ILE | H | 8 | −11.999 | 35.579 | 105.880 | 1.00 | 43.37 | O |
| ATOM | 24830 | N | THR | H | 9 | −9.828 | 36.088 | 106.219 | 1.00 | 55.57 | N |
| ATOM | 24832 | CA | THR | H | 9 | −9.628 | 35.093 | 107.286 | 1.00 | 52.77 | C |
| ATOM | 24834 | CB | THR | H | 9 | −8.126 | 34.835 | 107.589 | 1.00 | 49.75 | C |
| ATOM | 24836 | OG1 | THR | H | 9 | −7.386 | 36.053 | 107.637 | 1.00 | 46.63 | O |
| ATOM | 24838 | CG2 | THR | H | 9 | −7.426 | 34.052 | 106.487 | 1.00 | 51.02 | C |
| ATOM | 24842 | C | THR | H | 9 | −10.365 | 35.497 | 108.570 | 1.00 | 49.79 | C |
| ATOM | 24843 | O | THR | H | 9 | −10.509 | 34.715 | 109.507 | 1.00 | 57.33 | O |
| ATOM | 24844 | N | ASP | H | 10 | −10.943 | 36.684 | 108.543 | 1.00 | 46.48 | N |
| ATOM | 24846 | CA | ASP | H | 10 | −11.928 | 37.078 | 109.526 | 1.00 | 46.15 | C |
| ATOM | 24848 | CB | ASP | H | 10 | −12.018 | 38.602 | 109.605 | 1.00 | 50.06 | C |
| ATOM | 24851 | CG | ASP | H | 10 | −10.698 | 39.251 | 110.073 | 1.00 | 67.01 | C |
| ATOM | 24852 | OD1 | ASP | H | 10 | −9.814 | 38.525 | 110.588 | 1.00 | 84.09 | O |
| ATOM | 24853 | OD2 | ASP | H | 10 | −10.443 | 40.472 | 109.952 | 1.00 | 53.93 | O |
| ATOM | 24854 | C | ASP | H | 10 | −13.297 | 36.478 | 109.320 | 1.00 | 48.64 | C |
| ATOM | 24855 | O | ASP | H | 10 | −14.075 | 36.435 | 110.256 | 1.00 | 50.03 | O |
| ATOM | 24856 | N | TYR | H | 11 | −13.608 | 36.021 | 108.110 | 1.00 | 55.93 | N |
| ATOM | 24858 | CA | TYR | H | 11 | −14.920 | 35.433 | 107.821 | 1.00 | 49.24 | C |
| ATOM | 24860 | CB | TYR | H | 11 | −15.536 | 36.160 | 106.650 | 1.00 | 39.96 | C |
| ATOM | 24863 | CG | TYR | H | 11 | −15.561 | 37.660 | 106.852 | 1.00 | 44.57 | C |
| ATOM | 24864 | CD1 | TYR | H | 11 | −14.461 | 38.449 | 106.556 | 1.00 | 45.66 | C |
| ATOM | 24866 | CE1 | TYR | H | 11 | −14.497 | 39.828 | 106.712 | 1.00 | 32.31 | C |
| ATOM | 24868 | CZ | TYR | H | 11 | −15.652 | 40.443 | 107.148 | 1.00 | 62.14 | C |
| ATOM | 24869 | OH | TYR | H | 11 | −15.713 | 41.830 | 107.334 | 1.00 | 69.39 | O |
| ATOM | 24871 | CE2 | TYR | H | 11 | −16.763 | 39.665 | 107.417 | 1.00 | 52.85 | C |
| ATOM | 24873 | CD2 | TYR | H | 11 | −16.698 | 38.296 | 107.306 | 1.00 | 40.27 | C |
| ATOM | 24875 | C | TYR | H | 11 | −14.830 | 33.932 | 107.553 | 1.00 | 52.36 | C |
| ATOM | 24876 | O | TYR | H | 11 | −15.157 | 33.139 | 108.417 | 1.00 | 65.85 | O |
| ATOM | 24877 | N | PHE | H | 12 | −14.233 | 33.560 | 106.432 | 1.00 | 50.09 | N |
| ATOM | 24879 | CA | PHE | H | 12 | −13.787 | 32.206 | 106.140 | 1.00 | 43.84 | C |
| ATOM | 24881 | CB | PHE | H | 12 | −13.396 | 32.151 | 104.657 | 1.00 | 44.05 | C |
| ATOM | 24884 | CG | PHE | H | 12 | −14.368 | 32.903 | 103.763 | 1.00 | 45.15 | C |
| ATOM | 24885 | CD1 | PHE | H | 12 | −15.568 | 33.380 | 104.277 | 1.00 | 27.56 | C |
| ATOM | 24887 | CE1 | PHE | H | 12 | −16.453 | 34.088 | 103.488 | 1.00 | 35.61 | C |
| ATOM | 24889 | CZ | PHE | H | 12 | −16.157 | 34.359 | 102.171 | 1.00 | 36.31 | C |
| ATOM | 24891 | CE2 | PHE | H | 12 | −14.950 | 33.944 | 101.661 | 1.00 | 56.16 | C |
| ATOM | 24893 | CD2 | PHE | H | 12 | −14.056 | 33.216 | 102.454 | 1.00 | 43.02 | C |
| ATOM | 24895 | C | PHE | H | 12 | −12.689 | 31.591 | 107.005 | 1.00 | 47.14 | C |
| ATOM | 24896 | O | PHE | H | 12 | −11.645 | 32.180 | 107.281 | 1.00 | 54.33 | O |
| ATOM | 24897 | N | HIS | H | 13 | −12.919 | 30.335 | 107.371 | 1.00 | 53.22 | N |
| ATOM | 24899 | CA | HIS | H | 13 | −12.044 | 29.616 | 108.289 | 1.00 | 54.59 | C |
| ATOM | 24901 | CB | HIS | H | 13 | −12.669 | 29.492 | 109.678 | 1.00 | 58.81 | C |
| ATOM | 24904 | CG | HIS | H | 13 | −12.795 | 30.795 | 110.403 | 1.00 | 65.33 | C |
| ATOM | 24905 | ND1 | HIS | H | 13 | −14.006 | 31.429 | 110.583 | 1.00 | 61.79 | N |
| ATOM | 24907 | CE1 | HIS | H | 13 | −13.820 | 32.547 | 111.262 | 1.00 | 81.61 | C |
| ATOM | 24909 | NE2 | HIS | H | 13 | −12.531 | 32.656 | 111.534 | 1.00 | 96.38 | N |
| ATOM | 24911 | CD2 | HIS | H | 13 | −11.865 | 31.577 | 111.002 | 1.00 | 77.28 | C |
| ATOM | 24913 | C | HIS | H | 13 | −11.843 | 28.232 | 107.767 | 1.00 | 50.90 | C |
| ATOM | 24914 | O | HIS | H | 13 | −12.646 | 27.717 | 106.994 | 1.00 | 55.36 | O |
| ATOM | 24915 | N | PRO | H | 14 | −10.737 | 27.649 | 108.192 | 1.00 | 59.74 | N |
| ATOM | 24916 | CA | PRO | H | 14 | −10.191 | 26.443 | 107.553 | 1.00 | 64.32 | C |
| ATOM | 24918 | CB | PRO | H | 14 | −8.857 | 26.232 | 108.273 | 1.00 | 64.42 | C |
| ATOM | 24921 | CG | PRO | H | 14 | −8.560 | 27.532 | 108.998 | 1.00 | 65.49 | C |
| ATOM | 24924 | CD | PRO | H | 14 | −9.878 | 28.210 | 109.250 | 1.00 | 56.37 | C |
| ATOM | 24927 | C | PRO | H | 14 | −11.091 | 25.218 | 107.713 | 1.00 | 75.44 | C |
| ATOM | 24928 | O | PRO | H | 14 | −11.622 | 25.076 | 108.812 | 1.00 | 78.27 | O |
| ATOM | 24929 | N | LYS | H | 15 | −11.213 | 24.366 | 106.686 | 1.00 | 85.72 | N |
| ATOM | 24931 | CA | LYS | H | 15 | −12.217 | 23.292 | 106.586 | 1.00 | 88.09 | C |
| ATOM | 24933 | CB | LYS | H | 15 | −12.032 | 22.528 | 105.269 | 1.00 | 88.10 | C |
| ATOM | 24936 | CG | LYS | H | 15 | −12.981 | 22.877 | 104.121 | 1.00 | 90.10 | C |
| ATOM | 24939 | CD | LYS | H | 15 | −12.565 | 22.093 | 102.869 | 1.00 | 100.73 | C |
| ATOM | 24942 | CE | LYS | H | 15 | −13.449 | 22.371 | 101.658 | 1.00 | 103.18 | C |
| ATOM | 24945 | NZ | LYS | H | 15 | −14.548 | 23.330 | 101.956 | 1.00 | 107.79 | N |
| ATOM | 24949 | C | LYS | H | 15 | −12.180 | 22.258 | 107.722 | 1.00 | 94.08 | C |
| ATOM | 24950 | O | LYS | H | 15 | −13.214 | 21.937 | 108.320 | 1.00 | 96.04 | O |
| ATOM | 24951 | N | LYS | H | 16 | −11.005 | 21.687 | 107.977 | 1.00 | 100.00 | N |
| ATOM | 24953 | CA | LYS | H | 16 | −10.876 | 20.594 | 108.947 | 1.00 | 106.34 | C |
| ATOM | 24955 | CB | LYS | H | 16 | −11.350 | 19.253 | 108.356 | 1.00 | 107.30 | C |
| ATOM | 24958 | CG | LYS | H | 16 | −12.764 | 18.834 | 108.762 | 1.00 | 112.09 | C |
| ATOM | 24961 | CD | LYS | H | 16 | −13.138 | 19.297 | 110.176 | 1.00 | 110.26 | C |
| ATOM | 24964 | CE | LYS | H | 16 | −14.623 | 19.644 | 110.295 | 1.00 | 110.39 | C |
| ATOM | 24967 | NZ | LYS | H | 16 | −15.081 | 20.609 | 109.248 | 1.00 | 105.19 | N |
| ATOM | 24971 | C | LYS | H | 16 | −9.432 | 20.454 | 109.427 | 1.00 | 107.51 | C |
| ATOM | 24972 | O | LYS | H | 16 | −9.052 | 21.155 | 110.364 | 1.00 | 105.76 | O |
| ATOM | 24973 | OXT | LYS | H | 16 | −8.662 | 19.650 | 108.889 | 1.00 | 107.80 | O |
| ATOM | 24974 | N | SER | J | 1 | 25.298 | 24.847 | 84.333 | 1.00 | 123.86 | N |
| ATOM | 24976 | CA | SER | J | 1 | 25.217 | 24.359 | 82.923 | 1.00 | 127.51 | C |
| ATOM | 24978 | CB | SER | J | 1 | 24.554 | 22.977 | 82.894 | 1.00 | 126.21 | C |
| ATOM | 24981 | OG | SER | J | 1 | 25.533 | 21.948 | 82.975 | 1.00 | 116.46 | O |
| ATOM | 24983 | C | SER | J | 1 | 24.484 | 25.330 | 81.980 | 1.00 | 128.67 | C |
| ATOM | 24984 | O | SER | J | 1 | 23.266 | 25.226 | 81.802 | 1.00 | 129.21 | O |
| ATOM | 24987 | N | ALA | J | 2 | 25.240 | 26.247 | 81.366 | 1.00 | 129.36 | N |
| ATOM | 24989 | CA | ALA | J | 2 | 24.762 | 27.109 | 80.267 | 1.00 | 128.99 | C |
| ATOM | 24991 | CB | ALA | J | 2 | 24.117 | 28.391 | 80.851 | 1.00 | 125.13 | C |
| ATOM | 24995 | C | ALA | J | 2 | 25.803 | 27.469 | 79.156 | 1.00 | 127.25 | C |
| ATOM | 24996 | O | ALA | J | 2 | 25.729 | 28.552 | 78.564 | 1.00 | 133.25 | O |
| ATOM | 24997 | N | VAL | J | 3 | 26.739 | 26.562 | 78.851 | 1.00 | 119.66 | N |
| ATOM | 24999 | CA | VAL | J | 3 | 27.663 | 26.651 | 77.703 | 1.00 | 112.29 | C |
| ATOM | 25001 | CB | VAL | J | 3 | 26.944 | 26.357 | 76.335 | 1.00 | 114.73 | C |
| ATOM | 25003 | CG1 | VAL | J | 3 | 27.471 | 25.078 | 75.682 | 1.00 | 110.07 | C |
| ATOM | 25007 | CG2 | VAL | J | 3 | 25.441 | 26.187 | 76.512 | 1.00 | 120.91 | C |
| ATOM | 25011 | C | VAL | J | 3 | 28.582 | 27.904 | 77.703 | 1.00 | 103.01 | C |
| ATOM | 25012 | O | VAL | J | 3 | 28.892 | 28.428 | 78.770 | 1.00 | 97.92 | O |
| ATOM | 25013 | N | LEU | J | 4 | 29.099 | 28.329 | 76.549 | 1.00 | 91.90 | N |
| ATOM | 25015 | CA | LEU | J | 4 | 30.072 | 29.429 | 76.473 | 1.00 | 85.11 | C |
| ATOM | 25017 | CB | LEU | J | 4 | 31.487 | 28.849 | 76.517 | 1.00 | 81.66 | C |
| ATOM | 25020 | CG | LEU | J | 4 | 32.609 | 29.768 | 77.015 | 1.00 | 95.73 | C |
| ATOM | 25022 | CD1 | LEU | J | 4 | 33.605 | 29.039 | 77.955 | 1.00 | 100.70 | C |
| ATOM | 25026 | CD2 | LEU | J | 4 | 33.330 | 30.404 | 75.824 | 1.00 | 81.89 | C |
| ATOM | 25030 | C | LEU | J | 4 | 29.898 | 30.336 | 75.219 | 1.00 | 79.01 | C |
| ATOM | 25031 | O | LEU | J | 4 | 29.663 | 29.833 | 74.113 | 1.00 | 74.23 | O |
| ATOM | 25032 | N | GLN | J | 5 | 29.973 | 31.663 | 75.374 | 1.00 | 64.26 | N |
| ATOM | 25034 | CA | GLN | J | 5 | 29.596 | 32.549 | 74.276 | 1.00 | 59.27 | C |
| ATOM | 25036 | CB | GLN | J | 5 | 28.884 | 33.825 | 74.736 | 1.00 | 59.34 | C |
| ATOM | 25039 | CG | GLN | J | 5 | 28.502 | 34.791 | 73.576 | 1.00 | 46.89 | C |
| ATOM | 25042 | CD | GLN | J | 5 | 27.231 | 34.344 | 72.842 | 1.00 | 62.17 | C |
| ATOM | 25043 | OE1 | GLN | J | 5 | 26.124 | 34.396 | 73.392 | 1.00 | 73.53 | O |
| ATOM | 25044 | NE2 | GLN | J | 5 | 27.394 | 33.868 | 71.615 | 1.00 | 38.86 | N |
| ATOM | 25047 | C | GLN | J | 5 | 30.784 | 32.937 | 73.392 | 1.00 | 61.06 | C |
| ATOM | 25048 | O | GLN | J | 5 | 31.648 | 33.719 | 73.785 | 1.00 | 63.50 | O |
| ATOM | 25049 | N | LYS | J | 6 | 30.776 | 32.456 | 72.154 | 1.00 | 55.31 | N |
| ATOM | 25051 | CA | LYS | J | 6 | 31.752 | 32.873 | 71.180 | 1.00 | 53.33 | C |
| ATOM | 25053 | CB | LYS | J | 6 | 31.756 | 31.876 | 70.013 | 1.00 | 55.90 | C |
| ATOM | 25056 | CG | LYS | J | 6 | 32.402 | 30.515 | 70.339 | 1.00 | 73.38 | C |
| ATOM | 25059 | CD | LYS | J | 6 | 33.549 | 30.610 | 71.376 | 1.00 | 88.10 | C |
| ATOM | 25062 | CE | LYS | J | 6 | 34.818 | 29.872 | 70.953 | 1.00 | 84.47 | C |
| ATOM | 25065 | NZ | LYS | J | 6 | 35.354 | 30.367 | 69.648 | 1.00 | 81.16 | N |
| ATOM | 25069 | C | LYS | J | 6 | 31.440 | 34.284 | 70.695 | 1.00 | 55.85 | C |
| ATOM | 25070 | O | LYS | J | 6 | 30.485 | 34.957 | 71.115 | 1.00 | 55.25 | O |
| ATOM | 25071 | N | LYS | J | 7 | 32.299 | 34.728 | 69.799 | 1.00 | 52.22 | N |
| ATOM | 25073 | CA | LYS | J | 7 | 32.312 | 36.097 | 69.353 | 1.00 | 49.53 | C |
| ATOM | 25075 | CB | LYS | J | 7 | 33.480 | 36.814 | 70.056 | 1.00 | 45.45 | C |
| ATOM | 25078 | CG | LYS | J | 7 | 33.095 | 38.018 | 70.933 | 1.00 | 52.63 | C |
| ATOM | 25081 | CD | LYS | J | 7 | 33.245 | 37.754 | 72.425 | 1.00 | 66.72 | C |
| ATOM | 25084 | CE | LYS | J | 7 | 32.592 | 38.846 | 73.265 | 1.00 | 73.31 | C |
| ATOM | 25087 | NZ | LYS | J | 7 | 33.586 | 39.562 | 74.110 | 1.00 | 70.80 | N |
| ATOM | 25091 | C | LYS | J | 7 | 32.506 | 36.013 | 67.828 | 1.00 | 50.89 | C |
| ATOM | 25092 | O | LYS | J | 7 | 33.203 | 35.139 | 67.317 | 1.00 | 53.09 | O |
| ATOM | 25093 | N | ILE | J | 8 | 31.845 | 36.893 | 67.090 | 1.00 | 51.52 | N |
| ATOM | 25095 | CA | ILE | J | 8 | 32.033 | 37.005 | 65.647 | 1.00 | 49.72 | C |
| ATOM | 25097 | CB | ILE | J | 8 | 31.206 | 38.225 | 65.138 | 1.00 | 56.11 | C |
| ATOM | 25099 | CG1 | ILE | J | 8 | 29.706 | 37.917 | 65.204 | 1.00 | 58.92 | C |
| ATOM | 25102 | CD1 | ILE | J | 8 | 29.165 | 37.061 | 64.065 | 1.00 | 62.04 | C |
| ATOM | 25106 | CG2 | ILE | J | 8 | 31.613 | 38.676 | 63.731 | 1.00 | 55.39 | C |
| ATOM | 25110 | C | ILE | J | 8 | 33.512 | 37.138 | 65.258 | 1.00 | 49.69 | C |
| ATOM | 25111 | O | ILE | J | 8 | 33.946 | 36.526 | 64.276 | 1.00 | 42.57 | O |
| ATOM | 25112 | N | THR | J | 9 | 34.278 | 37.942 | 66.000 | 1.00 | 49.00 | N |
| ATOM | 25114 | CA | THR | J | 9 | 35.731 | 38.074 | 65.780 | 1.00 | 55.12 | C |
| ATOM | 25116 | CB | THR | J | 9 | 36.421 | 38.934 | 66.842 | 1.00 | 54.72 | C |
| ATOM | 25118 | OG1 | TER | J | 9 | 36.314 | 38.278 | 68.124 | 1.00 | 46.84 | O |
| ATOM | 25120 | CG2 | THR | J | 9 | 35.815 | 40.324 | 66.987 | 1.00 | 59.96 | C |
| ATOM | 25124 | C | THR | J | 9 | 36.471 | 36.736 | 65.871 | 1.00 | 54.71 | C |
| ATOM | 25125 | O | THR | J | 9 | 37.631 | 36.659 | 65.468 | 1.00 | 59.27 | O |
| ATOM | 25126 | N | ASP | J | 10 | 35.842 | 35.721 | 66.455 | 1.00 | 45.69 | N |
| ATOM | 25128 | CA | ASP | J | 10 | 36.418 | 34.392 | 66.438 | 1.00 | 45.71 | C |
| ATOM | 25130 | CB | ASP | J | 10 | 35.786 | 33.496 | 67.499 | 1.00 | 44.68 | C |
| ATOM | 25133 | CG | ASP | J | 10 | 36.002 | 34.015 | 68.872 | 1.00 | 45.22 | C |
| ATOM | 25134 | OD1 | ASP | J | 10 | 36.907 | 34.881 | 68.993 | 1.00 | 63.83 | O |
| ATOM | 25135 | OD2 | ASP | J | 10 | 35.302 | 33.642 | 69.848 | 1.00 | 52.57 | O |
| ATOM | 25136 | C | ASP | J | 10 | 36.297 | 33.681 | 65.097 | 1.00 | 51.19 | C |
| ATOM | 25137 | O | ASP | J | 10 | 37.051 | 32.752 | 64.870 | 1.00 | 58.49 | O |
| ATOM | 25138 | N | TYR | J | 11 | 35.330 | 34.040 | 64.252 | 1.00 | 52.16 | N |
| ATOM | 25140 | CA | TYR | J | 11 | 35.174 | 33.389 | 62.953 | 1.00 | 47.78 | C |
| ATOM | 25142 | CB | TYR | J | 11 | 33.747 | 32.876 | 62.816 | 1.00 | 42.22 | C |
| ATOM | 25145 | CG | TYR | J | 11 | 33.235 | 32.126 | 64.027 | 1.00 | 54.40 | C |
| ATOM | 25146 | CD1 | TYR | J | 11 | 32.765 | 32.791 | 65.172 | 1.00 | 47.35 | C |
| ATOM | 25148 | CE1 | TYR | J | 11 | 32.343 | 32.082 | 66.290 | 1.00 | 42.23 | C |
| ATOM | 25150 | CZ | TYR | J | 11 | 32.344 | 30.689 | 66.238 | 1.00 | 56.21 | C |
| ATOM | 25151 | OH | TYR | J | 11 | 31.899 | 29.866 | 67.261 | 1.00 | 51.58 | O |
| ATOM | 25153 | CE2 | TYR | J | 11 | 32.808 | 30.039 | 65.117 | 1.00 | 47.60 | C |
| ATOM | 25155 | CD2 | TYR | J | 11 | 33.216 | 30.745 | 64.019 | 1.00 | 44.72 | C |
| ATOM | 25157 | C | TYR | J | 11 | 35.490 | 34.330 | 61.783 | 1.00 | 54.49 | C |
| ATOM | 25158 | O | TYR | J | 11 | 35.772 | 33.874 | 60.690 | 1.00 | 62.64 | O |
| ATOM | 25159 | N | PHE | J | 12 | 35.344 | 35.635 | 61.980 | 1.00 | 61.23 | N |
| ATOM | 25161 | CA | PHE | J | 12 | 35.506 | 36.608 | 60.901 | 1.00 | 64.84 | C |
| ATOM | 25163 | CB | PHE | J | 12 | 34.247 | 37.457 | 60.664 | 1.00 | 67.90 | C |
| ATOM | 25166 | CG | PHE | J | 12 | 33.025 | 36.667 | 60.299 | 1.00 | 71.76 | C |
| ATOM | 25167 | CD1 | PHE | J | 12 | 31.772 | 37.097 | 60.721 | 1.00 | 63.88 | C |
| ATOM | 25169 | CE1 | PHE | J | 12 | 30.632 | 36.396 | 60.392 | 1.00 | 55.32 | C |
| ATOM | 25171 | CZ | PHE | J | 12 | 30.716 | 35.306 | 59.599 | 1.00 | 36.09 | C |
| ATOM | 25173 | CE2 | PHE | J | 12 | 31.960 | 34.838 | 59.195 | 1.00 | 65.68 | C |
| ATOM | 25175 | CD2 | PHE | J | 12 | 33.116 | 35.509 | 59.548 | 1.00 | 54.82 | C |
| ATOM | 25177 | C | PHE | J | 12 | 36.631 | 37.566 | 61.259 | 1.00 | 64.06 | C |
| ATOM | 25178 | O | PHE | J | 12 | 36.645 | 38.149 | 62.355 | 1.00 | 60.54 | O |
| ATOM | 25179 | N | HIS | J | 13 | 37.547 | 37.745 | 60.312 | 1.00 | 64.02 | N |
| ATOM | 26181 | CA | HIS | J | 13 | 38.733 | 38.539 | 60.571 | 1.00 | 69.82 | C |
| ATOM | 25183 | CB | HIS | J | 13 | 39.943 | 37.639 | 60.450 | 1.00 | 68.36 | C |
| ATOM | 25186 | CG | HIS | J | 13 | 39.820 | 36.398 | 61.277 | 1.00 | 73.60 | C |
| ATOM | 25187 | ND1 | HIS | J | 13 | 40.305 | 36.314 | 62.566 | 1.00 | 63.10 | N |
| ATOM | 25189 | CE1 | HIS | J | 13 | 40.016 | 35.125 | 63.064 | 1.00 | 70.96 | C |
| ATOM | 25191 | NE2 | HIS | J | 13 | 39.290 | 34.466 | 62.176 | 1.00 | 74.19 | N |
| ATOM | 25193 | CD2 | HIS | J | 13 | 39.153 | 35.240 | 61.050 | 1.00 | 74.37 | C |
| ATOM | 25195 | C | HIS | J | 13 | 38.814 | 39.720 | 59.624 | 1.00 | 74.13 | C |
| ATOM | 25196 | O | HIS | J | 13 | 38.191 | 39.718 | 58.554 | 1.00 | 78.17 | O |
| ATOM | 25197 | N | PRO | J | 14 | 39.530 | 40.757 | 60.038 | 1.00 | 72.03 | N |
| ATOM | 25198 | CA | PRO | J | 14 | 39.647 | 41.945 | 59.198 | 1.00 | 77.92 | C |
| ATOM | 25200 | CB | PRO | J | 14 | 40.790 | 42.722 | 59.846 | 1.00 | 71.23 | C |
| ATOM | 25203 | CG | PRO | J | 14 | 40.704 | 42.352 | 61.256 | 1.00 | 65.29 | C |
| ATOM | 25206 | CD | PRO | J | 14 | 40.265 | 40.902 | 61.301 | 1.00 | 70.45 | C |
| ATOM | 25209 | C | PRO | J | 14 | 39.944 | 41.557 | 57.751 | 1.00 | 90.04 | C |
| ATOM | 25210 | O | PRO | J | 14 | 40.491 | 40.481 | 57.471 | 1.00 | 97.59 | O |
| ATOM | 25211 | N | LYS | J | 15 | 39.538 | 42.437 | 56.841 | 1.00 | 98.98 | N |
| ATOM | 25213 | CA | LYS | J | 15 | 39.716 | 42.256 | 55.403 | 1.00 | 100.62 | C |
| ATOM | 25215 | CB | LYS | J | 15 | 38.575 | 42.992 | 54.670 | 1.00 | 101.15 | C |
| ATOM | 25218 | CG | LYS | J | 15 | 37.665 | 42.142 | 53.797 | 1.00 | 96.07 | C |
| ATOM | 25221 | CD | LYS | J | 15 | 36.463 | 42.936 | 53.279 | 1.00 | 101.80 | C |
| ATOM | 25224 | CE | LYS | J | 15 | 35.888 | 42.373 | 51.962 | 1.00 | 99.49 | C |
| ATOM | 25227 | NZ | LYS | J | 15 | 34.695 | 41.460 | 52.092 | 1.00 | 83.30 | N |
| ATOM | 25231 | C | LYS | J | 15 | 41.083 | 42.801 | 54.935 | 1.00 | 102.18 | C |
| ATOM | 25232 | O | LYS | J | 15 | 41.421 | 42.653 | 53.766 | 1.00 | 106.52 | O |
| ATOM | 25233 | N | LYS | J | 16 | 41.850 | 43.448 | 55.816 | 1.00 | 103.60 | N |
| ATOM | 25235 | CA | LYS | J | 16 | 43.162 | 43.993 | 55.466 | 1.00 | 104.40 | C |
| ATOM | 25237 | CB | LYS | J | 16 | 43.035 | 45.458 | 55.023 | 1.00 | 106.10 | C |
| ATOM | 25240 | CG | LYS | J | 16 | 42.382 | 45.711 | 53.644 | 1.00 | 111.90 | C |
| ATOM | 25243 | CD | LYS | J | 16 | 40.883 | 46.077 | 53.686 | 1.00 | 105.05 | C |
| ATOM | 25246 | CE | LYS | J | 16 | 40.606 | 47.490 | 54.180 | 1.00 | 104.34 | C |
| ATOM | 25249 | NZ | LYS | J | 16 | 41.119 | 48.487 | 53.214 | 1.00 | 109.04 | N |
| ATOM | 25253 | C | LYS | J | 16 | 44.104 | 43.913 | 56.672 | 1.00 | 108.05 | C |
| ATOM | 25254 | O | LYS | J | 16 | 44.196 | 42.971 | 57.469 | 1.00 | 103.92 | O |
| ATOM | 25255 | OXT | LYS | J | 16 | 44.877 | 44.831 | 56.939 | 1.00 | 114.31 | O |
| ATOM | 25256 | N | SER | L | 1 | 20.135 | 86.110 | 64.253 | 1.00 | 119.03 | N |
| ATOM | 25258 | CA | SER | L | 1 | 20.822 | 84.965 | 64.926 | 1.00 | 120.80 | C |
| ATOM | 25260 | CB | SER | L | 1 | 22.182 | 85.396 | 65.509 | 1.00 | 120.52 | C |
| ATOM | 25263 | OG | SER | L | 1 | 22.327 | 85.060 | 66.883 | 1.00 | 113.56 | O |
| ATOM | 25265 | C | SER | L | 1 | 19.929 | 84.258 | 65.965 | 1.00 | 121.45 | C |
| ATOM | 25266 | O | SER | L | 1 | 19.837 | 83.031 | 65.937 | 1.00 | 128.20 | O |
| ATOM | 25269 | N | ALA | L | 2 | 19.258 | 85.001 | 66.851 | 1.00 | 117.10 | N |
| ATOM | 25271 | CA | ALA | L | 2 | 18.022 | 84.544 | 67.522 | 1.00 | 110.12 | C |
| ATOM | 25273 | CB | ALA | L | 2 | 17.126 | 83.778 | 66.558 | 1.00 | 108.19 | C |
| ATOM | 25277 | C | ALA | L | 2 | 18.249 | 83.722 | 68.800 | 1.00 | 106.14 | C |
| ATOM | 25278 | O | ALA | L | 2 | 18.107 | 82.494 | 68.806 | 1.00 | 98.39 | O |
| ATOM | 25279 | N | VAL | L | 3 | 18.594 | 84.424 | 69.880 | 1.00 | 100.69 | N |
| ATOM | 25281 | CA | VAL | L | 3 | 19.359 | 83.848 | 70.987 | 1.00 | 93.82 | C |
| ATOM | 25283 | CB | VAL | L | 3 | 20.677 | 84.632 | 71.285 | 1.00 | 91.09 | C |
| ATOM | 25285 | CG1 | VAL | L | 3 | 21.889 | 83.752 | 71.066 | 1.00 | 88.22 | C |
| ATOM | 25289 | CG2 | VAL | L | 3 | 20.791 | 85.896 | 70.433 | 1.00 | 96.85 | C |
| ATOM | 25293 | C | VAL | L | 3 | 18.459 | 83.889 | 72.215 | 1.00 | 86.56 | C |
| ATOM | 25294 | O | VAL | L | 3 | 18.353 | 82.898 | 72.946 | 1.00 | 82.54 | O |
| ATOM | 25295 | N | LEU | L | 4 | 17.790 | 85.030 | 72.390 | 1.00 | 76.44 | N |
| ATOM | 25297 | CA | LEU | L | 4 | 17.139 | 85.377 | 73.646 | 1.00 | 72.70 | C |
| ATOM | 25299 | CB | LEU | L | 4 | 17.775 | 86.641 | 74.254 | 1.00 | 77.26 | C |
| ATOM | 25302 | CG | LEU | L | 4 | 17.226 | 88.038 | 73.930 | 1.00 | 86.59 | C |
| ATOM | 25304 | CD1 | LEU | L | 4 | 16.213 | 88.493 | 74.974 | 1.00 | 87.39 | C |
| ATOM | 25308 | CD2 | LEU | L | 4 | 18.350 | 89.070 | 73.823 | 1.00 | 96.61 | C |
| ATOM | 25312 | C | LEU | L | 4 | 15.595 | 85.424 | 73.624 | 1.00 | 61.83 | C |
| ATOM | 25313 | O | LEU | L | 4 | 14.937 | 86.137 | 72.848 | 1.00 | 58.36 | O |
| ATOM | 25314 | N | GLN | L | 5 | 15.020 | 84.627 | 74.513 | 1.00 | 49.45 | N |
| ATOM | 25316 | CA | GLN | L | 5 | 13.579 | 84.386 | 74.540 | 1.00 | 50.22 | C |
| ATOM | 25318 | CB | GLN | L | 5 | 13.308 | 83.052 | 75.239 | 1.00 | 47.68 | C |
| ATOM | 25321 | CG | GLN | L | 5 | 11.855 | 82.821 | 75.523 | 1.00 | 54.10 | C |
| ATOM | 25324 | CD | GLN | L | 5 | 11.133 | 82.284 | 74.328 | 1.00 | 62.32 | C |
| ATOM | 25325 | OE1 | GLN | L | 5 | 11.526 | 81.220 | 73.774 | 1.00 | 41.92 | O |
| ATOM | 25326 | NE2 | GLN | L | 5 | 10.089 | 83.025 | 73.905 | 1.00 | 40.93 | N |
| ATOM | 25329 | C | GLN | L | 5 | 12.807 | 85.468 | 75.272 | 1.00 | 40.81 | C |
| ATOM | 25330 | O | GLN | L | 5 | 12.992 | 85.618 | 76.440 | 1.00 | 43.25 | O |
| ATOM | 25331 | N | LYS | L | 6 | 11.920 | 86.198 | 74.615 | 1.00 | 45.56 | N |
| ATOM | 25333 | CA | LYS | L | 6 | 11.252 | 87.339 | 75.243 | 1.00 | 42.13 | C |
| ATOM | 25335 | CB | LYS | L | 6 | 10.600 | 88.227 | 74.170 | 1.00 | 45.52 | C |
| ATOM | 25338 | CG | LYS | L | 6 | 11.568 | 89.157 | 73.407 | 1.00 | 56.13 | C |
| ATOM | 25341 | CD | LYS | L | 6 | 12.205 | 90.242 | 74.305 | 1.00 | 79.48 | C |
| ATOM | 25344 | CE | LYS | L | 6 | 11.590 | 91.648 | 74.093 | 1.00 | 81.75 | C |
| ATOM | 25347 | NZ | LYS | L | 6 | 11.909 | 92.622 | 75.190 | 1.00 | 70.01 | N |
| ATOM | 25351 | C | LYS | L | 6 | 10.144 | 86.776 | 76.108 | 1.00 | 43.84 | C |
| ATOM | 25352 | O | LYS | L | 6 | 9.980 | 85.564 | 76.150 | 1.00 | 48.30 | O |
| ATOM | 25353 | N | LYS | L | 7 | 9.309 | 87.645 | 76.669 | 1.00 | 45.16 | N |
| ATOM | 25355 | CA | LYS | L | 7 | 8.174 | 87.220 | 77.490 | 1.00 | 50.91 | C |
| ATOM | 25357 | CB | LYS | L | 7 | 8.466 | 87.446 | 78.977 | 1.00 | 51.89 | C |
| ATOM | 25360 | CG | LYS | L | 7 | 8.803 | 86.184 | 79.729 | 1.00 | 59.90 | C |
| ATOM | 25363 | CD | LYS | L | 7 | 9.895 | 86.426 | 80.756 | 1.00 | 53.35 | C |
| ATOM | 25366 | CE | LYS | L | 7 | 10.358 | 85.113 | 81.414 | 1.00 | 38.83 | C |
| ATOM | 25369 | NZ | LYS | L | 7 | 10.977 | 85.472 | 82.706 | 1.00 | 44.16 | N |
| ATOM | 25373 | C | LYS | L | 7 | 6.873 | 87.943 | 77.125 | 1.00 | 51.19 | C |
| ATOM | 25374 | O | LYS | L | 7 | 6.880 | 89.088 | 76.685 | 1.00 | 57.23 | O |
| ATOM | 25375 | N | ILE | L | 8 | 5.738 | 87.277 | 77.278 | 1.00 | 46.41 | N |
| ATOM | 25377 | CA | ILE | L | 8 | 4.538 | 87.850 | 76.730 | 1.00 | 46.42 | C |
| ATOM | 25379 | CB | ILE | L | 8 | 3.381 | 86.843 | 76.848 | 1.00 | 40.95 | C |
| ATOM | 25381 | CG1 | ILE | L | 8 | 3.044 | 86.298 | 75.464 | 1.00 | 55.21 | C |
| ATOM | 25384 | CD1 | ILE | L | 8 | 1.865 | 85.363 | 75.451 | 1.00 | 65.24 | C |
| ATOM | 25388 | CG2 | ILE | L | 8 | 2.117 | 87.565 | 77.234 | 1.00 | 58.22 | C |
| ATOM | 25392 | C | ILE | L | 8 | 4.251 | 89.187 | 77.440 | 1.00 | 46.02 | C |
| ATOM | 25393 | O | ILE | L | 8 | 3.647 | 90.106 | 76.883 | 1.00 | 38.76 | O |
| ATOM | 25394 | N | THR | L | 9 | 4.648 | 89.284 | 78.702 | 1.00 | 43.90 | N |
| ATOM | 25396 | CA | THR | L | 9 | 4.438 | 90.516 | 79.427 | 1.00 | 46.17 | C |
| ATOM | 25398 | CB | THR | L | 9 | 4.871 | 90.340 | 80.885 | 1.00 | 41.25 | C |
| ATOM | 25400 | OG1 | THR | L | 9 | 6.224 | 89.905 | 80.931 | 1.00 | 52.37 | O |
| ATOM | 25402 | CG2 | THR | L | 9 | 4.141 | 89.239 | 81.566 | 1.00 | 39.63 | C |
| ATOM | 25406 | C | THR | L | 9 | 5.226 | 91.685 | 78.797 | 1.00 | 49.11 | C |
| ATOM | 25407 | O | THR | L | 9 | 4.929 | 92.845 | 79.054 | 1.00 | 54.61 | O |
| ATOM | 25408 | N | ASP | L | 10 | 6.216 | 91.408 | 77.958 | 1.00 | 46.22 | N |
| ATOM | 25410 | CA | ASP | L | 10 | 6.843 | 92.474 | 77.189 | 1.00 | 45.39 | C |
| ATOM | 25412 | CB | ASP | L | 10 | 8.216 | 92.064 | 76.638 | 1.00 | 38.97 | C |
| ATOM | 25415 | CG | ASP | L | 10 | 9.159 | 91.494 | 77.698 | 1.00 | 54.62 | C |
| ATOM | 25416 | OD1 | ASP | L | 10 | 9.014 | 91.757 | 78.921 | 1.00 | 50.98 | O |
| ATOM | 25417 | OD2 | ASP | L | 10 | 10.125 | 90.761 | 77.369 | 1.00 | 43.70 | O |
| ATOM | 25418 | C | ASP | L | 10 | 5.988 | 93.038 | 76.052 | 1.00 | 44.14 | C |
| ATOM | 25419 | O | ASP | L | 10 | 6.247 | 94.132 | 75.607 | 1.00 | 64.66 | O |
| ATOM | 25420 | N | TYR | L | 11 | 5.014 | 92.317 | 75.522 | 1.00 | 51.92 | N |
| ATOM | 25422 | CA | TYR | L | 11 | 4.125 | 92.850 | 74.480 | 1.00 | 50.48 | C |
| ATOM | 25424 | CB | TYR | L | 11 | 4.053 | 91.867 | 73.302 | 1.00 | 47.12 | C |
| ATOM | 25427 | CG | TYR | L | 11 | 5.411 | 91.326 | 72.873 | 1.00 | 51.82 | C |
| ATOM | 25428 | CD1 | TYR | L | 11 | 6.084 | 90.350 | 73.606 | 1.00 | 51.75 | C |
| ATOM | 25430 | CE1 | TYR | L | 11 | 7.345 | 89.889 | 73.218 | 1.00 | 44.42 | C |
| ATOM | 25432 | CZ | TYR | L | 11 | 7.937 | 90.391 | 72.066 | 1.00 | 47.29 | C |
| ATOM | 25433 | OH | TYR | L | 11 | 9.161 | 89.940 | 71.608 | 1.00 | 57.00 | O |
| ATOM | 25435 | CE2 | TYR | L | 11 | 7.286 | 91.345 | 71.328 | 1.00 | 48.43 | C |
| ATOM | 25437 | CD2 | TYR | L | 11 | 6.021 | 91.787 | 71.715 | 1.00 | 53.20 | C |
| ATOM | 25439 | C | TYR | L | 11 | 2.702 | 93.172 | 75.004 | 1.00 | 55.98 | C |
| ATOM | 25440 | O | TYR | L | 11 | 2.075 | 94.141 | 74.567 | 1.00 | 57.76 | O |
| ATOM | 25441 | N | PHE | L | 12 | 2.178 | 92.369 | 75.929 | 1.00 | 55.63 | N |
| ATOM | 25443 | CA | PHE | L | 12 | 0.865 | 92.647 | 76.496 | 1.00 | 58.85 | C |
| ATOM | 25445 | CB | PHE | L | 12 | −0.045 | 91.419 | 76.449 | 1.00 | 55.30 | C |
| ATOM | 25448 | CG | PHE | L | 12 | −0.192 | 90.808 | 75.089 | 1.00 | 51.60 | C |
| ATOM | 25449 | CD1 | PHE | L | 12 | 0.084 | 91.518 | 73.950 | 1.00 | 51.59 | C |
| ATOM | 25451 | CE1 | PHE | L | 12 | −0.033 | 90.930 | 72.717 | 1.00 | 45.81 | C |
| ATOM | 25453 | CZ | PHE | L | 12 | −0.443 | 89.631 | 72.613 | 1.00 | 39.31 | C |
| ATOM | 25455 | CE2 | PHE | L | 12 | −0.687 | 88.907 | 73.729 | 1.00 | 42.17 | C |
| ATOM | 25457 | CD2 | PHE | L | 12 | −0.597 | 89.498 | 74.959 | 1.00 | 50.98 | C |
| ATOM | 25459 | C | PHE | L | 12 | 0.998 | 93.090 | 77.950 | 1.00 | 65.72 | C |
| ATOM | 25460 | O | PHE | L | 12 | 1.823 | 92.565 | 78.700 | 1.00 | 67.84 | O |
| ATOM | 25461 | N | HIS | L | 13 | 0.114 | 94.004 | 78.346 | 1.00 | 69.41 | N |
| ATOM | 25463 | CA | HIS | L | 13 | 0.174 | 94.699 | 79.623 | 1.00 | 67.91 | C |
| ATOM | 25465 | CB | HIS | L | 13 | 0.455 | 96.165 | 79.318 | 1.00 | 69.98 | C |
| ATOM | 25468 | CG | HIS | L | 13 | 1.825 | 96.417 | 78.763 | 1.00 | 83.72 | C |
| ATOM | 25469 | ND1 | HIS | L | 13 | 2.055 | 96.662 | 77.424 | 1.00 | 92.43 | N |
| ATOM | 25471 | CE1 | HIS | L | 13 | 3.341 | 96.892 | 77.231 | 1.00 | 90.20 | C |
| ATOM | 25473 | NE2 | HIS | L | 13 | 3.957 | 96.794 | 78.397 | 1.00 | 96.73 | N |
| ATOM | 25475 | CD2 | HIS | L | 13 | 3.032 | 96.510 | 79.373 | 1.00 | 91.10 | C |
| ATOM | 25477 | C | HIS | L | 13 | −1.192 | 94.607 | 80.294 | 1.00 | 68.28 | C |
| ATOM | 25478 | O | HIS | L | 13 | −2.201 | 94.654 | 79.605 | 1.00 | 75.88 | O |
| ATOM | 25479 | N | PRO | L | 14 | −1.254 | 94.591 | 81.619 | 1.00 | 67.63 | N |
| ATOM | 25480 | CA | PRO | L | 14 | −2.517 | 94.394 | 82.347 | 1.00 | 67.59 | C |
| ATOM | 25482 | CB | PRO | L | 14 | −2.092 | 94.491 | 83.818 | 1.00 | 70.01 | C |
| ATOM | 25485 | CG | PRO | L | 14 | −0.776 | 95.188 | 83.839 | 1.00 | 59.42 | C |
| ATOM | 25488 | CD | PRO | L | 14 | −0.134 | 94.905 | 82.520 | 1.00 | 71.41 | C |
| ATOM | 25491 | C | PRO | L | 14 | −3.509 | 95.501 | 82.023 | 1.00 | 71.26 | C |
| ATOM | 25492 | O | PRO | L | 14 | −3.079 | 96.436 | 81.354 | 1.00 | 73.84 | O |
| ATOM | 25493 | N | LYS | L | 15 | −4.773 | 95.419 | 82.434 | 1.00 | 80.35 | N |
| ATOM | 25495 | CA | LYS | L | 15 | −5.864 | 95.841 | 81.541 | 1.00 | 94.39 | C |
| ATOM | 25497 | CB | LYS | L | 15 | −7.040 | 94.843 | 81.518 | 1.00 | 96.86 | C |
| ATOM | 25500 | CG | LYS | L | 15 | −7.969 | 94.955 | 80.283 | 1.00 | 93.88 | C |
| ATOM | 25503 | CD | LYS | L | 15 | −8.765 | 93.655 | 80.058 | 1.00 | 110.12 | C |
| ATOM | 25506 | CE | LYS | L | 15 | −10.119 | 93.827 | 79.343 | 1.00 | 112.35 | C |
| ATOM | 25509 | NZ | LYS | L | 15 | −10.434 | 95.240 | 78.982 | 1.00 | 116.39 | N |
| ATOM | 25513 | C | LYS | L | 15 | −6.396 | 97.264 | 81.730 | 1.00 | 101.41 | C |
| ATOM | 25514 | O | LYS | L | 15 | −6.910 | 97.859 | 80.769 | 1.00 | 105.73 | O |
| ATOM | 25515 | N | LYS | L | 16 | −6.284 | 97.808 | 82.942 | 1.00 | 107.03 | N |
| ATOM | 25517 | CA | LYS | L | 16 | −6.377 | 99.265 | 83.149 | 1.00 | 114.30 | C |
| ATOM | 25519 | CB | LYS | L | 16 | −5.655 | 100.031 | 82.012 | 1.00 | 113.17 | C |
| ATOM | 25522 | CG | LYS | L | 16 | −4.513 | 100.976 | 82.442 | 1.00 | 115.12 | C |
| ATOM | 25525 | CD | LYS | L | 16 | −4.982 | 102.052 | 83.451 | 1.00 | 121.90 | C |
| ATOM | 25528 | CE | LYS | L | 16 | −4.077 | 103.280 | 83.515 | 1.00 | 112.97 | C |
| ATOM | 25531 | NZ | LYS | L | 16 | −3.979 | 103.915 | 82.177 | 1.00 | 116.05 | N |
| ATOM | 25535 | C | LYS | L | 16 | −7.832 | 99.776 | 83.368 | 1.00 | 115.61 | C |
| ATOM | 25536 | O | LYS | L | 16 | −8.866 | 99.099 | 83.272 | 1.00 | 115.87 | O |
| ATOM | 25537 | OXT | LYS | L | 16 | −8.160 | 100.918 | 83.730 | 1.00 | 110.23 | O |
| ATOM | 25538 | OW0 | HOH | W | 1 | 12.109 | 52.944 | 44.524 | 1.00 | 28.64 | O |
| ATOM | 25541 | OW0 | HOH | W | 2 | 8.377 | 39.779 | 49.819 | 1.00 | 26.62 | O |
| ATOM | 25544 | OW0 | HOH | W | 3 | −32.442 | 71.527 | 80.253 | 1.00 | 41.13 | O |
| ATOM | 25547 | OW0 | HOH | W | 5 | 0.413 | 55.816 | 57.343 | 1.00 | 31.48 | O |
| ATOM | 25550 | OW0 | HOH | W | 6 | 3.627 | 41.768 | 2.975 | 1.00 | 26.81 | O |
| ATOM | 25553 | OW0 | HOH | W | 8 | −29.145 | 75.750 | 32.186 | 1.00 | 35.23 | O |
| ATOM | 25556 | OW0 | HOH | W | 9 | −29.770 | 64.026 | 18.205 | 1.00 | 31.21 | O |
| ATOM | 25559 | OW0 | HOH | W | 10 | 13.364 | 63.523 | 46.964 | 1.00 | 26.27 | O |
| ATOM | 25562 | OW0 | HOH | W | 12 | 13.826 | 48.941 | 44.710 | 1.00 | 40.53 | O |
| ATOM | 25565 | OW0 | HOH | W | 13 | −3.102 | 34.750 | 69.132 | 1.00 | 52.01 | O |
| ATOM | 25568 | OW0 | HOH | W | 14 | −15.910 | 59.693 | −8.202 | 1.00 | 20.85 | O |
| ATOM | 25571 | OW0 | HOH | W | 15 | −11.750 | 10.911 | 84.827 | 1.00 | 47.92 | O |
| ATOM | 25574 | OW0 | HOH | W | 16 | −13.308 | 59.902 | 34.983 | 1.00 | 35.36 | O |
| ATOM | 25577 | OW0 | HOH | W | 17 | −0.643 | 35.166 | 42.868 | 1.00 | 43.37 | O |
| ATOM | 25580 | OW0 | HOH | W | 18 | −4.748 | 48.715 | −24.537 | 1.00 | 54.86 | O |
| ATOM | 25583 | OW0 | HOH | W | 19 | −32.122 | 75.247 | −14.180 | 1.00 | 64.95 | O |
| ATOM | 25586 | OW0 | HOH | W | 20 | 5.716 | 52.582 | 27.656 | 1.00 | 27.81 | O |
| ATOM | 25589 | OW0 | HOH | W | 21 | −14.779 | 38.656 | −16.489 | 1.00 | 49.73 | O |
| ATOM | 25592 | OW0 | HOH | W | 22 | 6.939 | 14.271 | 86.535 | 1.00 | 41.41 | O |
| ATOM | 25595 | OW0 | HOH | W | 23 | 9.409 | 81.305 | 71.790 | 1.00 | 51.90 | O |
| ATOM | 25598 | OW0 | HOH | W | 24 | −27.021 | 68.341 | −2.196 | 1.00 | 32.10 | O |
| ATOM | 25601 | OW0 | HOH | W | 25 | −43.230 | 69.885 | 18.802 | 1.00 | 39.20 | O |
| ATOM | 25604 | OW0 | HOH | W | 26 | 16.471 | 31.378 | 83.591 | 1.00 | 34.78 | O |
| ATOM | 25607 | OW0 | HOH | W | 27 | 35.749 | 34.501 | 72.370 | 1.00 | 56.46 | O |
| ATOM | 25610 | OW0 | HOH | W | 28 | −5.967 | 86.873 | 66.321 | 1.00 | 42.48 | O |
| ATOM | 25613 | OW0 | HOH | W | 29 | 1.160 | 28.816 | 18.450 | 1.00 | 43.73 | O |
| ATOM | 25616 | OW0 | HOH | W | 30 | −40.980 | 76.622 | 24.472 | 1.00 | 55.04 | O |
| ATOM | 25619 | OW0 | HOH | W | 31 | −14.812 | 47.975 | −5.488 | 1.00 | 51.86 | O |
| ATOM | 25622 | OW0 | HOH | W | 32 | 13.266 | 46.749 | 44.241 | 1.00 | 39.66 | O |
| ATOM | 25625 | OW0 | HOH | W | 33 | 12.441 | 43.886 | 35.818 | 1.00 | 44.00 | O |
| ATOM | 25628 | OW0 | HOH | W | 34 | −23.913 | 45.273 | −7.400 | 1.00 | 42.04 | O |
| ATOM | 25631 | OW0 | HOH | W | 35 | 11.803 | 38.262 | 61.582 | 1.00 | 31.84 | O |
| ATOM | 25634 | OW0 | HOH | W | 36 | −21.550 | 76.258 | 12.757 | 1.00 | 44.73 | O |
| ATOM | 25637 | OW0 | HOH | W | 37 | −9.332 | 53.077 | −5.409 | 1.00 | 33.90 | O |
| ATOM | 25640 | OW0 | HOH | W | 38 | 26.737 | 53.566 | 39.673 | 1.00 | 33.99 | O |
| ATOM | 25643 | OW0 | HOH | W | 39 | 0.602 | 59.489 | 45.119 | 1.00 | 46.58 | O |
| ATOM | 25646 | OW0 | HOH | W | 40 | 1.522 | 29.480 | 68.479 | 1.00 | 47.49 | O |
| ATOM | 25649 | OW0 | HOH | W | 41 | −43.073 | 82.578 | 34.586 | 1.00 | 42.26 | O |
| ATOM | 25652 | OW0 | HOH | W | 42 | −4.502 | 31.289 | 13.758 | 1.00 | 47.01 | O |
| ATOM | 25655 | OW0 | HOH | W | 43 | −10.254 | 62.987 | 36.404 | 1.00 | 34.14 | O |
| ATOM | 25658 | OW0 | HOH | W | 44 | −6.206 | 82.884 | 84.899 | 1.00 | 42.67 | O |
| ATOM | 25661 | OW0 | HOH | W | 45 | −12.748 | 23.973 | 73.944 | 1.00 | 48.54 | O |
| ATOM | 25664 | OW0 | HOH | W | 46 | 7.020 | 42.961 | 50.622 | 1.00 | 44.40 | O |
| ATOM | 25667 | OW0 | HOH | W | 47 | 12.452 | 89.157 | 78.122 | 1.00 | 62.22 | O |
| ATOM | 25670 | OW0 | HOH | W | 48 | 22.449 | 36.605 | 76.544 | 1.00 | 51.58 | O |
| ATOM | 25673 | OW0 | HOH | W | 49 | −3.368 | 45.193 | 16.696 | 1.00 | 52.58 | O |
| ATOM | 25676 | OW0 | HOH | W | 50 | 11.946 | 50.338 | 45.300 | 1.00 | 34.37 | O |
| ATOM | 25679 | OW0 | HOH | W | 51 | −22.861 | 70.582 | −0.942 | 1.00 | 55.67 | O |
| ATOM | 25682 | OW0 | HOH | W | 52 | −20.462 | 27.003 | 75.631 | 1.00 | 56.14 | O |
| ATOM | 25685 | OW0 | HOH | W | 53 | 24.525 | 33.682 | 70.859 | 1.00 | 53.40 | O |
| ATOM | 25688 | OW0 | HOH | W | 54 | −5.018 | 35.670 | 107.876 | 1.00 | 48.76 | O |
| ATOM | 25691 | OW0 | HOH | W | 55 | −26.450 | 80.459 | 93.786 | 1.00 | 47.35 | O |
| ATOM | 25694 | OW0 | HOH | W | 56 | −25.524 | 57.165 | −21.997 | 1.00 | 38.83 | O |
| ATOM | 25697 | OW0 | HOH | W | 57 | −27.281 | 45.890 | 105.052 | 1.00 | 53.78 | O |
| ATOM | 25700 | OW0 | HOH | W | 58 | −34.974 | 60.066 | −18.044 | 1.00 | 51.05 | O |
| ATOM | 25703 | OW0 | HOH | W | 59 | −9.013 | 84.137 | 83.002 | 1.00 | 50.04 | O |
| ATOM | 25706 | OW0 | HOH | W | 61 | −20.716 | 41.547 | 100.546 | 1.00 | 48.94 | O |
| ATOM | 25709 | OW0 | HOH | W | 62 | −13.466 | 72.928 | 64.798 | 1.00 | 45.91 | O |
| ATOM | 25712 | OW0 | HOH | W | 63 | 9.712 | 53.300 | 22.706 | 1.00 | 42.12 | O |
| ATOM | 25715 | OW0 | HOH | W | 64 | −15.296 | 43.620 | 84.931 | 1.00 | 26.16 | O |
| ATOM | 25718 | OW0 | HOH | W | 65 | −40.598 | 66.798 | 7.469 | 1.00 | 50.70 | O |
| ATOM | 25721 | OW0 | HOH | W | 67 | −14.581 | 45.901 | −28.332 | 1.00 | 42.66 | O |
| ATOM | 25724 | OW0 | HOH | W | 68 | 3.203 | 33.866 | 64.445 | 1.00 | 57.42 | O |
| ATOM | 25727 | OW0 | HOH | W | 69 | 7.306 | 56.416 | 34.382 | 1.00 | 40.14 | O |
| ATOM | 25730 | OW0 | HOH | W | 70 | −44.578 | 65.846 | 40.740 | 1.00 | 59.57 | O |
| ATOM | 25733 | OW0 | HOH | W | 71 | −21.735 | 62.531 | 26.573 | 1.00 | 46.23 | O |
| ATOM | 25736 | OW0 | HOH | W | 72 | 0.364 | 89.031 | 68.829 | 1.00 | 57.57 | O |
| ATOM | 25739 | OW0 | HOH | W | 73 | −47.375 | 83.776 | −0.144 | 1.00 | 48.16 | O |
| ATOM | 25742 | OW0 | HOH | W | 74 | −41.341 | 49.683 | 93.427 | 1.00 | 61.73 | O |
| ATOM | 25745 | OW0 | HOH | W | 75 | 8.360 | 30.553 | −19.621 | 1.00 | 48.41 | O |
| ATOM | 25748 | OW0 | HOH | W | 76 | −8.635 | 37.931 | 81.365 | 1.00 | 33.34 | O |
| ATOM | 25751 | OW0 | HOH | W | 77 | −25.028 | 66.153 | 81.899 | 1.00 | 68.75 | O |
| ATOM | 25754 | OW0 | HOH | W | 78 | −3.921 | 61.465 | 44.310 | 1.00 | 48.27 | O |
| ATOM | 25757 | OW0 | HOH | W | 79 | 6.539 | 50.572 | 12.663 | 1.00 | 42.32 | O |
| ATOM | 25760 | OW0 | HOH | W | 80 | −46.228 | 55.526 | 32.068 | 1.00 | 58.90 | O |
| ATOM | 25763 | OW0 | HOH | W | 81 | 11.056 | 45.107 | 51.310 | 1.00 | 53.80 | O |
| ATOM | 25766 | OW0 | HOH | W | 82 | −38.597 | 61.010 | 12.674 | 1.00 | 64.51 | O |
| ATOM | 25769 | OW0 | HOH | W | 83 | 3.707 | 58.128 | 56.631 | 1.00 | 48.71 | O |
| ATOM | 25772 | OW0 | HOH | W | 85 | 5.476 | 92.035 | 60.186 | 1.00 | 43.93 | O |
| ATOM | 25775 | OW0 | HOH | W | 86 | 12.454 | 79.360 | 57.939 | 1.00 | 45.23 | O |
| ATOM | 25778 | OW0 | HOH | W | 87 | 18.151 | 46.104 | 65.562 | 1.00 | 45.58 | O |
| ATOM | 25781 | OW0 | HOH | W | 88 | −30.087 | 79.736 | 34.828 | 1.00 | 41.11 | O |
| ATOM | 25784 | OW0 | HOH | W | 89 | −1.967 | 71.447 | 65.788 | 1.00 | 44.08 | O |
| ATOM | 25787 | OW0 | HOH | W | 91 | 26.296 | 25.379 | 69.625 | 1.00 | 51.53 | O |
| ATOM | 25790 | OW0 | HOH | W | 92 | −3.271 | 63.928 | 60.403 | 1.00 | 46.56 | O |
| ATOM | 25793 | OW0 | HOH | W | 93 | 28.075 | 32.485 | 58.938 | 1.00 | 51.37 | O |
| ATOM | 25796 | OW0 | HOH | W | 99 | 1.803 | 49.060 | −26.181 | 1.00 | 45.95 | O |
| ATOM | 25799 | OW0 | HOH | W | 100 | 7.308 | 44.100 | 1.185 | 1.00 | 34.98 | O |
| ATOM | 25802 | OW0 | HOH | W | 102 | −12.956 | 70.389 | 62.289 | 1.00 | 37.43 | O |
| ATOM | 25805 | OW0 | HOH | W | 103 | −31.795 | 66.045 | −13.523 | 1.00 | 44.57 | O |
| ATOM | 25808 | OW0 | HOH | W | 104 | −9.987 | 31.358 | −17.589 | 1.00 | 40.99 | O |
| ATOM | 25811 | OW0 | HOH | W | 105 | −33.061 | 51.418 | −8.640 | 1.00 | 38.99 | O |
| ATOM | 25814 | OW0 | HOH | W | 106 | −17.142 | 14.182 | 86.008 | 1.00 | 58.13 | O |
| ATOM | 25817 | OW0 | HOH | W | 107 | −21.297 | 27.776 | 87.387 | 1.00 | 42.22 | O |
| ATOM | 25820 | OW0 | HOH | W | 108 | −1.948 | 49.120 | 24.418 | 1.00 | 32.27 | O |
| ATOM | 25823 | OW0 | HOH | W | 110 | 25.684 | 29.356 | 61.555 | 1.00 | 45.74 | O |
| ATOM | 25826 | OW0 | HOH | W | 111 | 3.814 | 54.199 | 26.638 | 1.00 | 46.09 | O |
| ATOM | 25829 | OW0 | HOH | W | 114 | −8.411 | 41.684 | 110.673 | 1.00 | 51.83 | O |
| ATOM | 25832 | OW0 | HOH | W | 115 | 9.697 | 26.599 | 19.428 | 1.00 | 58.61 | O |
| ATOM | 25835 | OW0 | HOH | W | 117 | 4.571 | 52.103 | 11.791 | 1.00 | 38.04 | O |
| ATOM | 25838 | OW0 | HOH | W | 118 | −21.994 | 79.840 | −9.065 | 1.00 | 53.69 | O |
| ATOM | 25841 | OW0 | HOH | W | 119 | 14.867 | 88.916 | 65.712 | 1.00 | 50.15 | O |
| ATOM | 25844 | OW0 | HOH | W | 120 | −18.118 | 39.182 | −17.672 | 1.00 | 50.04 | O |
| ATOM | 25847 | OW0 | HOH | W | 122 | 30.752 | 60.331 | 45.333 | 1.00 | 66.75 | O |
| ATOM | 25850 | OW0 | HOH | W | 123 | −7.993 | 84.654 | 33.308 | 1.00 | 90.09 | O |
| ATOM | 25853 | OW0 | HOH | W | 124 | −10.318 | 83.248 | 92.201 | 1.00 | 47.07 | O |
| ATOM | 25856 | OW0 | HOH | W | 125 | 13.577 | 77.164 | 49.745 | 1.00 | 58.29 | O |
| ATOM | 25859 | OW0 | HOH | W | 127 | 9.870 | 66.484 | 57.365 | 1.00 | 43.70 | O |
| ATOM | 25862 | OW0 | HOH | W | 128 | −49.657 | 76.948 | 0.508 | 1.00 | 60.27 | O |
| ATOM | 25865 | OW0 | HOH | W | 129 | 15.376 | 39.275 | 74.683 | 1.00 | 53.11 | O |
| ATOM | 25868 | OW0 | HOH | W | 130 | −4.057 | 47.935 | 21.274 | 1.00 | 34.02 | O |
| ATOM | 25871 | OW0 | HOH | W | 131 | 39.384 | 30.790 | 64.291 | 1.00 | 49.47 | O |
| ATOM | 25874 | OW0 | HOH | W | 132 | 8.660 | 44.068 | −7.240 | 1.00 | 44.26 | O |
| ATOM | 25877 | OW0 | HOH | W | 134 | −28.229 | 68.609 | 84.725 | 1.00 | 48.91 | O |
| ATOM | 25880 | OW0 | HOH | W | 136 | −47.199 | 71.126 | 15.171 | 1.00 | 52.89 | O |
| ATOM | 25883 | OW0 | HOH | W | 137 | 3.227 | 42.535 | 5.639 | 1.00 | 41.13 | O |
| ATOM | 25886 | OW0 | HOH | W | 139 | −22.393 | 89.325 | 80.020 | 1.00 | 56.86 | O |
| ATOM | 25889 | OW0 | HOH | W | 140 | −45.013 | 72.459 | 20.928 | 1.00 | 50.40 | O |
| ATOM | 25892 | OW0 | HOH | W | 141 | −50.581 | 76.924 | 11.065 | 1.00 | 52.29 | O |
| ATOM | 25895 | OW0 | HOH | W | 142 | −21.810 | 33.242 | 95.233 | 1.00 | 46.32 | O |
| ATOM | 25898 | OW0 | HOH | W | 143 | −28.788 | 61.709 | −27.174 | 1.00 | 47.90 | O |
| ATOM | 25901 | OW0 | HOH | W | 144 | −14.628 | 82.897 | 66.625 | 1.00 | 45.82 | O |
| ATOM | 25904 | OW0 | HOH | W | 145 | 21.310 | 71.058 | 69.982 | 1.00 | 46.10 | O |
| ATOM | 25907 | OW0 | HOH | W | 146 | −21.932 | 58.735 | 83.822 | 1.00 | 61.37 | O |
| ATOM | 25910 | OW0 | HOH | W | 147 | −6.624 | 65.615 | −13.587 | 1.00 | 62.11 | O |
| ATOM | 25913 | OW0 | HOH | W | 148 | −0.605 | 37.403 | 96.189 | 1.00 | 45.69 | O |
| ATOM | 25916 | OW0 | HOH | W | 149 | −0.889 | 15.418 | 94.905 | 1.00 | 49.63 | O |
| ATOM | 25919 | OW0 | HOH | W | 150 | −32.220 | 49.367 | −27.142 | 1.00 | 51.20 | O |
| ATOM | 25922 | OW0 | HOH | W | 151 | 12.611 | 55.653 | 32.363 | 1.00 | 50.39 | O |
| ATOM | 25925 | OW0 | HOH | W | 152 | −21.918 | 69.172 | 25.969 | 1.00 | 36.05 | O |
| ATOM | 25928 | OW0 | HOH | W | 154 | −23.865 | 82.801 | 88.096 | 1.00 | 52.05 | O |
| ATOM | 25931 | OW0 | HOH | W | 155 | 23.422 | 43.009 | 73.787 | 1.00 | 49.51 | O |
| ATOM | 25934 | OW0 | HOH | W | 156 | −17.871 | 58.231 | 85.473 | 1.00 | 62.36 | O |
| ATOM | 25937 | OW0 | HOH | W | 158 | 10.203 | 37.323 | −0.527 | 1.00 | 49.91 | O |
| ATOM | 25940 | OW0 | HOH | W | 159 | 6.367 | 64.420 | 56.566 | 1.00 | 53.49 | O |
| ATOM | 25943 | OW0 | HOH | W | 160 | 6.372 | 64.479 | 53.369 | 1.00 | 46.54 | O |
| ATOM | 25946 | OW0 | HOH | W | 161 | −27.932 | 50.479 | 99.845 | 1.00 | 58.09 | O |
| ATOM | 25949 | OW0 | HOH | W | 162 | −20.179 | 75.480 | 97.372 | 1.00 | 49.42 | O |
| ATOM | 25952 | OW0 | HOH | W | 164 | −36.557 | 73.108 | 34.954 | 1.00 | 50.97 | O |
| ATOM | 25955 | OW0 | HOH | W | 165 | 22.723 | 46.557 | 71.666 | 1.00 | 59.62 | O |
| ATOM | 25958 | OW0 | HOH | W | 166 | −48.682 | 76.115 | 9.352 | 1.00 | 55.37 | O |
| ATOM | 25961 | OW0 | HOH | W | 167 | 16.877 | 25.937 | 18.587 | 1.00 | 55.94 | O |
| ATOM | 25964 | OW0 | HOH | W | 168 | 14.299 | 64.063 | 71.118 | 1.00 | 58.44 | O |
| ATOM | 25967 | OW0 | HOH | W | 169 | −12.615 | 25.765 | 93.973 | 1.00 | 47.03 | O |
| ATOM | 25970 | OW0 | HOH | W | 170 | −3.313 | 62.496 | 25.319 | 1.00 | 60.89 | O |
| ATOM | 25973 | OW0 | HOH | W | 173 | −37.197 | 56.721 | −0.974 | 1.00 | 51.80 | O |
| ATOM | 25976 | OW0 | HOH | W | 175 | −14.410 | 14.123 | 77.755 | 1.00 | 53.82 | O |
| ATOM | 25979 | OW0 | HOH | W | 176 | 34.615 | 40.107 | 77.235 | 1.00 | 56.84 | O |
| ATOM | 25982 | OW0 | HOH | W | 177 | 18.284 | 29.460 | 78.462 | 1.00 | 68.70 | O |
| ATOM | 25985 | OW0 | HOH | W | 178 | 13.202 | 44.651 | 31.107 | 1.00 | 63.47 | O |
| ATOM | 25988 | OW0 | HOH | W | 179 | −21.015 | 37.857 | 92.954 | 1.00 | 51.53 | O |
| ATOM | 25991 | OW0 | HOH | W | 181 | −23.778 | 31.284 | 89.762 | 1.00 | 54.80 | O |
| ATOM | 25994 | OW0 | HOH | W | 182 | −0.261 | 30.494 | −16.235 | 1.00 | 52.58 | O |
| ATOM | 25997 | OW0 | HOH | W | 187 | −3.497 | 35.374 | 37.147 | 1.00 | 45.48 | O |
| ATOM | 26000 | OW0 | HOH | W | 188 | −26.913 | 42.841 | 104.702 | 1.00 | 62.32 | O |
| ATOM | 26003 | OW0 | HOH | W | 189 | 0.002 | 33.821 | −27.832 | 1.00 | 66.15 | O |
| ATOM | 26006 | OW0 | HOH | W | 190 | 9.383 | 47.385 | 52.436 | 1.00 | 46.87 | O |
| ATOM | 26009 | OW0 | HOH | W | 192 | 11.471 | 38.607 | 67.942 | 1.00 | 45.34 | O |
| ATOM | 26012 | OW0 | HOH | W | 194 | −1.705 | 47.715 | 16.520 | 1.00 | 49.79 | O |
| ATOM | 26015 | OW0 | HOH | W | 195 | −5.954 | 36.006 | 43.068 | 1.00 | 73.29 | O |
| ATOM | 26018 | OW0 | HOH | W | 196 | −3.486 | 46.696 | −23.310 | 1.00 | 36.56 | O |
| ATOM | 26021 | OW0 | HOH | W | 197 | 20.609 | 68.647 | 49.204 | 1.00 | 54.41 | O |
| ATOM | 26024 | OW0 | HOH | W | 200 | 13.911 | 24.855 | 14.618 | 1.00 | 49.41 | O |
| ATOM | 26027 | OW0 | HOH | W | 201 | 35.051 | 57.781 | 59.846 | 1.00 | 57.15 | O |
| ATOM | 26030 | OW0 | HOH | W | 202 | −36.793 | 59.044 | 0.959 | 1.00 | 51.83 | O |
| ATOM | 26033 | OW0 | HOH | W | 203 | 6.963 | 68.041 | 43.501 | 1.00 | 70.54 | O |
| ATOM | 26036 | OW0 | HOH | W | 204 | −4.130 | 49.108 | 55.339 | 1.00 | 55.32 | O |
| ATOM | 26039 | OW0 | HOH | W | 205 | −13.593 | 32.909 | 75.933 | 1.00 | 59.12 | O |
| ATOM | 26042 | OW0 | HOH | W | 206 | −8.082 | 86.730 | 38.464 | 1.00 | 63.69 | O |
| ATOM | 26045 | OW0 | HOH | W | 208 | −16.691 | 77.146 | −9.694 | 1.00 | 45.49 | O |
| ATOM | 26048 | OW0 | HOH | W | 209 | −29.510 | 65.033 | 20.587 | 1.00 | 48.41 | O |
| ATOM | 26051 | OW0 | HOH | W | 211 | 26.815 | 45.629 | 46.746 | 1.00 | 59.04 | O |
| ATOM | 26054 | OW0 | HOH | W | 212 | 0.634 | 50.318 | 3.466 | 1.00 | 48.47 | O |
| ATOM | 26057 | OW0 | HOH | W | 213 | −40.094 | 50.871 | 95.510 | 1.00 | 62.13 | O |
| ATOM | 26060 | OW0 | HOH | W | 214 | −33.614 | 55.320 | 23.044 | 1.00 | 56.41 | O |
| ATOM | 26063 | OW0 | HOH | W | 215 | 23.005 | 28.864 | 77.120 | 1.00 | 64.56 | O |
| ATOM | 26066 | OW0 | HOH | W | 218 | 3.166 | 54.369 | −14.156 | 1.00 | 55.19 | O |
| ATOM | 26069 | OW0 | HOH | W | 219 | 4.714 | 28.409 | 71.679 | 1.00 | 51.43 | O |
| ATOM | 26072 | OW0 | HOH | W | 220 | 13.804 | 36.836 | 27.726 | 1.00 | 62.35 | O |
| ATOM | 26075 | OW0 | HOH | W | 221 | 13.811 | 34.224 | 81.144 | 1.00 | 54.95 | O |
| ATOM | 26078 | OW0 | HOH | W | 222 | −16.576 | 85.446 | 91.748 | 1.00 | 56.17 | O |
| ATOM | 26081 | OW0 | HOH | W | 223 | −38.119 | 44.264 | 94.395 | 1.00 | 58.14 | O |
| ATOM | 26084 | OW0 | HOH | W | 227 | −29.025 | 52.221 | −14.013 | 1.00 | 42.96 | O |
| ATOM | 26087 | OW0 | HOH | W | 228 | −4.343 | 39.068 | 87.567 | 1.00 | 69.69 | O |
| ATOM | 26090 | OW0 | HOH | W | 229 | −2.372 | 70.703 | 77.668 | 1.00 | 58.08 | O |
| ATOM | 26093 | OW0 | HOH | W | 230 | 6.068 | 35.060 | 90.957 | 1.00 | 47.13 | O |
| ATOM | 26096 | OW0 | HOH | W | 232 | −14.488 | 52.389 | 25.193 | 1.00 | 54.78 | O |
| ATOM | 26099 | OW0 | HOH | W | 233 | 16.761 | 42.949 | 27.939 | 1.00 | 54.70 | O |
| ATOM | 26102 | OW0 | HOH | W | 236 | 11.191 | 44.174 | −10.324 | 1.00 | 50.98 | O |
| ATOM | 26105 | OW0 | HOH | W | 237 | −1.955 | 8.113 | 85.601 | 1.00 | 89.49 | O |
| ATOM | 26108 | OW0 | HOH | W | 239 | −23.964 | 70.413 | 73.397 | 1.00 | 78.84 | O |
| ATOM | 26111 | OW0 | HOH | W | 240 | −43.957 | 75.294 | 0.691 | 1.00 | 71.39 | O |
| ATOM | 26114 | OW0 | HOH | W | 241 | −1.236 | 30.818 | 34.665 | 1.00 | 69.50 | O |
| ATOM | 26117 | OW0 | HOH | W | 242 | −8.814 | 64.710 | −18.419 | 1.00 | 53.91 | O |
| ATOM | 26120 | OW0 | HOH | W | 243 | 6.005 | 89.885 | 84.539 | 1.00 | 62.77 | O |
| ATOM | 26123 | OW0 | HOH | W | 244 | −37.192 | 56.576 | −21.655 | 1.00 | 58.27 | O |
| ATOM | 26126 | OW0 | HOH | W | 246 | −15.514 | 28.906 | 107.421 | 1.00 | 48.65 | O |
| ATOM | 26129 | OW0 | HOH | W | 247 | 24.149 | 23.205 | 70.377 | 1.00 | 52.93 | O |
| ATOM | 26132 | OW0 | HOH | W | 248 | −6.501 | 56.114 | −21.712 | 1.00 | 46.97 | O |
| ATOM | 26135 | OW0 | HOH | W | 249 | −48.720 | 63.224 | 7.964 | 1.00 | 44.70 | O |
| ATOM | 26138 | OW0 | HOH | W | 251 | 13.075 | 52.713 | 37.089 | 1.00 | 55.66 | O |
| ATOM | 26141 | OW0 | HOH | W | 253 | −5.585 | 40.778 | 4.269 | 1.00 | 57.61 | O |
| ATOM | 26144 | OW0 | HOH | W | 254 | −33.487 | 61.390 | 34.543 | 1.00 | 60.37 | O |
| ATOM | 26147 | OW0 | HOH | W | 256 | 3.574 | 44.903 | 17.656 | 1.00 | 41.73 | O |
| ATOM | 26150 | OW0 | HOH | W | 257 | −6.688 | 29.747 | 14.071 | 1.00 | 56.56 | O |
| ATOM | 26153 | OW0 | HOH | W | 258 | −7.281 | 25.677 | 95.640 | 1.00 | 53.85 | O |
| ATOM | 26156 | OW0 | HOH | W | 260 | −11.098 | 19.048 | 91.351 | 1.00 | 48.65 | O |
| ATOM | 26159 | OW0 | HOH | W | 261 | 19.078 | 73.452 | 69.091 | 1.00 | 50.59 | O |
| ATOM | 26162 | OW0 | HOH | W | 262 | 10.100 | 22.725 | 14.491 | 1.00 | 70.47 | O |
| ATOM | 26165 | OW0 | HOH | W | 263 | 10.296 | 30.470 | 26.252 | 1.00 | 48.09 | O |
| ATOM | 26168 | OW0 | HOH | W | 264 | 36.397 | 47.977 | 63.767 | 1.00 | 50.90 | O |
| ATOM | 26171 | OW0 | HOH | W | 265 | −10.139 | 60.766 | −15.087 | 1.00 | 49.85 | O |
| ATOM | 26174 | OW0 | HOH | W | 266 | −31.035 | 49.020 | −7.401 | 1.00 | 45.48 | O |
| ATOM | 26177 | OW0 | HOH | W | 268 | −1.838 | 34.247 | 78.737 | 1.00 | 62.45 | O |
| ATOM | 26180 | OW0 | HOH | W | 271 | 20.357 | 33.271 | 56.076 | 1.00 | 52.47 | O |
| ATOM | 26183 | OW0 | HOH | W | 272 | 3.554 | 68.818 | 67.968 | 1.00 | 45.83 | O |
| ATOM | 26186 | OW0 | HOH | W | 273 | 18.063 | 30.466 | 80.889 | 1.00 | 47.26 | O |
| ATOM | 26189 | OW0 | HOH | W | 275 | −1.919 | 85.592 | 83.113 | 1.00 | 59.45 | O |
| ATOM | 26192 | OW0 | HOH | W | 276 | −1.087 | 31.984 | 101.587 | 1.00 | 37.88 | O |
| ATOM | 26195 | OW0 | HOH | W | 277 | −25.806 | 53.876 | −3.028 | 1.00 | 58.50 | O |
| ATOM | 26198 | OW0 | HOH | W | 279 | 35.128 | 59.321 | 52.474 | 1.00 | 58.43 | O |
| ATOM | 26201 | OW0 | HOH | W | 282 | −7.642 | 62.319 | 27.046 | 1.00 | 58.23 | O |
| ATOM | 26204 | OW0 | HOH | W | 283 | 13.384 | 53.835 | 43.615 | 1.00 | 38.88 | O |
| ATOM | 26207 | OW0 | HOH | W | 284 | 3.628 | 55.879 | −16.757 | 1.00 | 66.16 | O |
| ATOM | 26210 | OW0 | HOH | W | 286 | −46.842 | 62.430 | 29.813 | 1.00 | 61.99 | O |
| ATOM | 26213 | OW0 | HOH | W | 287 | −6.933 | 52.962 | 25.888 | 1.00 | 49.68 | O |
| ATOM | 26216 | OW0 | HOH | W | 289 | −13.526 | 46.240 | −23.707 | 1.00 | 66.19 | O |
| ATOM | 26219 | OW0 | HOH | W | 290 | −16.422 | 21.528 | 89.845 | 1.00 | 40.90 | O |
| ATOM | 26222 | OW0 | HOH | W | 292 | −11.157 | 60.663 | 30.763 | 1.00 | 77.68 | O |
| ATOM | 26225 | OW0 | HOH | W | 293 | 5.192 | 25.036 | 59.488 | 1.00 | 64.04 | O |
| ATOM | 26228 | OW0 | HOH | W | 295 | 8.031 | 24.369 | 12.215 | 1.00 | 47.90 | O |
| ATOM | 26231 | OW0 | HOH | W | 296 | −2.265 | 27.908 | 103.876 | 1.00 | 47.81 | O |
| ATOM | 26234 | OW0 | HOH | W | 297 | 19.299 | 35.871 | 75.781 | 1.00 | 54.73 | O |
| ATOM | 26237 | OW0 | HOH | W | 298 | −1.887 | 50.863 | 56.374 | 1.00 | 51.92 | O |
| ATOM | 26240 | OW0 | HOH | W | 300 | −27.611 | 42.193 | 83.010 | 1.00 | 57.83 | O |
| ATOM | 26243 | OW0 | HOH | W | 301 | 9.971 | 39.192 | 35.545 | 1.00 | 51.37 | O |
| ATOM | 26246 | OW0 | HOH | W | 303 | −18.457 | 79.375 | 21.265 | 1.00 | 50.81 | O |
| ATOM | 26249 | OW0 | HOH | W | 304 | −21.580 | 44.056 | −29.583 | 1.00 | 39.94 | O |
| ATOM | 26252 | OW0 | HOH | W | 305 | 9.290 | 31.225 | 7.187 | 1.00 | 50.11 | O |
| ATOM | 26255 | OW0 | HOH | W | 307 | 11.060 | 16.560 | 66.794 | 1.00 | 64.98 | O |
| ATOM | 26258 | OW0 | HOH | W | 308 | −25.520 | 72.827 | −3.418 | 1.00 | 47.82 | O |
| ATOM | 26261 | OW0 | HOH | W | 309 | −3.975 | 41.051 | 24.089 | 1.00 | 50.28 | O |
| ATOM | 26264 | OW0 | HOH | W | 310 | 30.559 | 50.354 | 60.553 | 1.00 | 33.19 | O |
| ATOM | 26267 | OW0 | HOH | W | 312 | −11.185 | 40.268 | −19.206 | 1.00 | 43.18 | O |
| ATOM | 26270 | OW0 | HOH | W | 313 | −7.310 | 48.172 | 101.918 | 1.00 | 49.83 | O |
| ATOM | 26273 | OW0 | HOH | W | 314 | 7.071 | 56.677 | 46.960 | 1.00 | 59.05 | O |
| ATOM | 26276 | OW0 | HOH | W | 317 | 26.445 | 68.171 | 40.721 | 1.00 | 59.49 | O |
| ATOM | 26279 | OW0 | HOH | W | 320 | −1.022 | 66.104 | 69.408 | 1.00 | 57.88 | O |
| ATOM | 26282 | OW0 | HOH | W | 324 | −33.020 | 62.938 | −23.530 | 1.00 | 57.64 | O |
| ATOM | 26285 | OW0 | HOH | W | 325 | −38.418 | 54.641 | 36.906 | 1.00 | 72.92 | O |
| ATOM | 26288 | OW0 | HOH | W | 326 | −17.696 | 66.140 | 47.370 | 1.00 | 89.97 | O |
| ATOM | 26291 | OW0 | HOH | W | 328 | 1.463 | 28.020 | 61.695 | 1.00 | 59.03 | O |
| ATOM | 26294 | OW0 | HOH | W | 329 | −9.977 | 24.228 | 95.247 | 1.00 | 51.85 | O |
| ATOM | 26297 | OW0 | HOH | W | 330 | 3.158 | 36.815 | 52.082 | 1.00 | 51.69 | O |
| ATOM | 26300 | OW0 | HOH | W | 332 | 27.199 | 21.116 | 60.348 | 1.00 | 50.00 | O |
| ATOM | 26303 | OW0 | HOH | W | 333 | 11.686 | 21.627 | 25.304 | 1.00 | 57.36 | O |
| ATOM | 26306 | OW0 | HOH | W | 335 | −15.311 | 84.174 | 68.704 | 1.00 | 58.88 | O |
| ATOM | 26309 | OW0 | HOH | W | 338 | −7.208 | 38.322 | 79.202 | 1.00 | 58.23 | O |
| ATOM | 26312 | OW0 | HOH | W | 339 | −25.399 | 47.725 | 104.507 | 1.00 | 49.49 | O |
| ATOM | 26315 | OW0 | HOH | W | 340 | 12.418 | 40.598 | 35.526 | 1.00 | 61.91 | O |
| ATOM | 26318 | OW0 | HOH | W | 343 | −5.896 | 29.502 | 34.357 | 1.00 | 48.59 | O |
| ATOM | 26321 | OW0 | HOH | W | 344 | 5.257 | 57.548 | 34.861 | 1.00 | 50.64 | O |
| ATOM | 26324 | OW0 | HOH | W | 345 | −8.451 | 78.647 | 63.422 | 1.00 | 50.10 | O |
| ATOM | 26327 | OW0 | HOH | W | 347 | −24.551 | 69.759 | 16.554 | 1.00 | 65.40 | O |
| ATOM | 26330 | OW0 | HOH | W | 349 | −13.014 | 48.898 | −2.116 | 1.00 | 41.96 | O |
| ATOM | 26333 | OW0 | HOH | W | 351 | −12.113 | 71.124 | 101.924 | 1.00 | 51.59 | O |
| ATOM | 26336 | OW0 | HOH | W | 352 | −33.393 | 70.308 | 38.420 | 1.00 | 80.24 | O |
| ATOM | 26339 | OW0 | HOH | W | 353 | −19.540 | 77.635 | 63.904 | 1.00 | 82.05 | O |
| ATOM | 26342 | OW0 | HOH | W | 354 | 8.137 | 27.363 | 52.909 | 1.00 | 111.13 | O |
| ATOM | 26345 | OW0 | HOH | W | 355 | −19.578 | 57.599 | 75.852 | 1.00 | 61.11 | O |
| ATOM | 26348 | OW0 | HOH | W | 357 | 34.003 | 30.566 | 60.181 | 1.00 | 53.68 | O |
| ATOM | 26351 | OW0 | HOH | W | 358 | −12.406 | 68.772 | −13.177 | 1.00 | 64.61 | O |
| ATOM | 26354 | OW0 | HOH | W | 359 | 6.781 | 50.004 | 15.341 | 1.00 | 53.47 | O |
| ATOM | 26357 | OW0 | HOH | W | 360 | −29.401 | 79.283 | 0.241 | 1.00 | 55.70 | O |
| ATOM | 26360 | OW0 | HOH | W | 361 | −46.131 | 86.647 | 10.528 | 1.00 | 54.53 | O |
| ATOM | 26363 | OW0 | HOH | W | 362 | 21.111 | 30.367 | 78.804 | 1.00 | 62.11 | O |
| ATOM | 26366 | OW0 | HOH | W | 363 | 1.292 | 60.268 | 29.459 | 1.00 | 56.64 | O |
| ATOM | 26369 | OW0 | HOH | W | 364 | −22.075 | 52.005 | −27.039 | 1.00 | 51.66 | O |
| ATOM | 26372 | OW0 | HOH | W | 365 | −12.039 | 46.373 | 100.781 | 1.00 | 55.76 | O |
| ATOM | 26375 | OW0 | HOH | W | 366 | −24.846 | 83.318 | 11.671 | 1.00 | 44.06 | O |
| ATOM | 26378 | OW0 | HOH | W | 367 | −24.709 | 27.826 | 80.877 | 1.00 | 54.19 | O |
| ATOM | 26381 | OW0 | HOH | W | 368 | 8.065 | 52.981 | −5.676 | 1.00 | 52.36 | O |
| ATOM | 26384 | OW0 | HOH | W | 369 | −24.730 | 50.622 | −3.492 | 1.00 | 60.83 | O |
| ATOM | 26387 | OW0 | HOH | W | 371 | 22.948 | 89.132 | 62.699 | 1.00 | 64.50 | O |
| ATOM | 26390 | OW0 | HOH | W | 372 | 11.179 | 45.026 | 23.506 | 1.00 | 60.24 | O |
| ATOM | 26393 | OW0 | HOH | W | 373 | 8.498 | 66.195 | 53.446 | 1.00 | 54.74 | O |
| ATOM | 26396 | OW0 | HOH | W | 374 | 19.828 | 45.119 | 19.240 | 1.00 | 53.46 | O |
| ATOM | 26399 | OW0 | HOH | W | 375 | −5.900 | 33.789 | 12.480 | 1.00 | 56.93 | O |
| ATOM | 26402 | OW0 | HOH | W | 376 | 10.025 | 39.155 | 50.596 | 1.00 | 58.49 | O |
| ATOM | 26405 | OW0 | HOH | W | 378 | −33.290 | 84.945 | 22.400 | 1.00 | 74.38 | O |
| ATOM | 26408 | OW0 | HOH | W | 379 | −11.805 | 62.457 | 25.178 | 1.00 | 57.11 | O |
| ATOM | 26411 | OW0 | HOH | W | 382 | −13.738 | 28.056 | 97.042 | 1.00 | 58.10 | O |
| ATOM | 26414 | OW0 | HOH | W | 383 | 16.041 | 71.490 | 48.169 | 1.00 | 64.48 | O |
| ATOM | 26417 | OW0 | HOH | W | 385 | −0.878 | 42.550 | −28.213 | 1.00 | 51.09 | O |
| ATOM | 26420 | OW0 | HOH | W | 386 | −0.995 | 22.356 | 98.130 | 1.00 | 44.71 | O |
| ATOM | 26423 | OW0 | HOH | W | 387 | 3.567 | 60.101 | 52.421 | 1.00 | 66.65 | O |
| ATOM | 26426 | OW0 | HOH | W | 388 | 9.060 | 42.135 | 47.638 | 1.00 | 52.32 | O |
| ATOM | 26429 | OW0 | HOH | W | 389 | −36.921 | 54.914 | −8.542 | 1.00 | 56.48 | O |
| ATOM | 26432 | OW0 | HOH | W | 391 | −15.340 | 40.624 | 77.864 | 1.00 | 63.59 | O |
| ATOM | 26435 | OW0 | HOH | W | 393 | −16.935 | 20.284 | 76.096 | 1.00 | 53.80 | O |
| ATOM | 26438 | OW0 | HOH | W | 394 | −17.615 | 48.957 | 77.405 | 1.00 | 64.08 | O |
| ATOM | 26441 | OW0 | HOH | W | 395 | −3.928 | 31.174 | 0.987 | 1.00 | 102.65 | O |
| ATOM | 26444 | OW0 | HOH | W | 396 | 0.328 | 22.371 | 96.040 | 1.00 | 49.29 | O |
| ATOM | 26447 | OW0 | HOH | W | 398 | −18.172 | 40.293 | −20.253 | 1.00 | 57.93 | O |
| ATOM | 26450 | OW0 | HOH | W | 399 | −4.658 | 36.347 | 67.856 | 1.00 | 69.97 | O |
| ATOM | 26453 | OW0 | HOH | W | 400 | −31.329 | 83.623 | 24.067 | 1.00 | 61.00 | O |
| ATOM | 26456 | OW0 | HOH | W | 402 | 12.199 | 74.203 | 72.534 | 1.00 | 41.91 | O |
| ATOM | 26459 | OW0 | HOH | W | 404 | −8.153 | 72.717 | 48.510 | 1.00 | 66.40 | O |
| ATOM | 26462 | OW0 | HOH | W | 405 | 6.139 | 70.130 | 74.751 | 1.00 | 63.45 | O |
| ATOM | 26465 | OW0 | HOH | W | 407 | −24.827 | 83.249 | 8.946 | 1.00 | 43.43 | O |
| ATOM | 26468 | OW0 | HOH | W | 408 | 11.947 | 65.263 | 71.304 | 1.00 | 62.13 | O |
| ATOM | 26471 | OW0 | HOH | W | 410 | 26.142 | 34.694 | 54.373 | 1.00 | 59.84 | O |
| ATOM | 26474 | OW0 | HOH | W | 411 | −28.217 | 50.904 | 83.559 | 1.00 | 69.84 | O |
| ATOM | 26477 | OW0 | HOH | W | 414 | −29.559 | 78.148 | 76.573 | 1.00 | 68.23 | O |
| ATOM | 26480 | OW0 | HOH | W | 418 | −3.578 | 45.068 | 5.516 | 1.00 | 61.17 | O |
| ATOM | 26483 | OW0 | HOH | W | 421 | 27.355 | 50.093 | 68.456 | 1.00 | 58.14 | O |
| ATOM | 26486 | OW0 | HOH | W | 422 | 27.870 | 45.033 | 54.224 | 1.00 | 99.04 | O |
| ATOM | 26489 | OW0 | HOH | W | 423 | −4.728 | 16.756 | 73.438 | 1.00 | 69.72 | O |
| ATOM | 26492 | O | HOH | W | 424 | −18.333 | 65.307 | 2.364 | 1.00 | 35.72 | O |
| ATOM | 26495 | O | HOH | W | 425 | −13.164 | 66.523 | −2.524 | 1.00 | 46.10 | O |
| ATOM | 26498 | O | HOH | W | 426 | 0.566 | 84.341 | 86.846 | 1.00 | 51.21 | O |
| ATOM | 26501 | O | HOH | W | 427 | −17.613 | 30.124 | 74.630 | 1.00 | 57.66 | O |
| ATOM | 26504 | O | HOH | W | 428 | −21.765 | 37.053 | −14.846 | 1.00 | 56.58 | O |
| ATOM | 26507 | O | HOH | W | 429 | −17.385 | 40.576 | 111.935 | 1.00 | 37.02 | O |
| ATOM | 26510 | O | HOH | W | 430 | 32.386 | 48.043 | 67.371 | 1.00 | 42.47 | O |
| ATOM | 26513 | O | HOH | W | 431 | 37.075 | 41.309 | 71.127 | 1.00 | 70.15 | O |
| ATOM | 26516 | O | HOH | W | 432 | 33.708 | 44.105 | 73.900 | 1.00 | 54.23 | O |
| ATOM | 26519 | O | HOH | W | 433 | 1.755 | 87.747 | 86.928 | 1.00 | 55.44 | O |

1. A crystal comprising human proliferating cell nuclear antigen (PCNA).

2. A crystal according to claim 1 which is human PCNA.

3. A crystal according to claim 2 which is monoclinic.

4. A crystal according to claim 2 which is of space group C121.

5. A crystal according to claim 3 wherein said crystal comprises a unit cell having the following unit dimensions: a=136.6 Å, b=83.26 Å, c=71.63 Å.

6. A crystal according to claim 3 having the atomic coordinates set forth in Table 3.

7. A crystal according to claim 2 which is trigonal.

8. A crystal according to claim 7 which is of space group P3.

9. A crystal according to claim 7 wherein said crystal comprises a unit cell having the following unit dimensions: a=82.89 Å, b=82.89 Å, c=70.86 Å.

10. A crystal according to claim 6 having the atomic coordinates set forth in Table 4.

11. A crystal according to claim 1 comprising human PCNA and a ligand.

12. A crystal according to claim 11 wherein the ligand is a peptide structurally related to p21.

13. A crystal according to claim 12 wherein the ligand is a peptide of formula I
(I)

| | |
|---|---|
| 1 16 | |
| SAVLQKKITDYFHPKK | (SEQ ID No. 4) |

14. A crystal according to claim 13 wherein said crystal comprises a unit cell having the following unit dimensions: a=119.1 Å, b=119.1 Å, c=305.82 Å.

15. A crystal according to claim 13 having the structural coordinates set forth in Table 5.

16. A crystal according to claim 1 which comprises a ligand binding domain.

17. A crystal comprising a human PCNA ligand binding domain.

18. A crystal according to claim 17 having a ligand associated therewith.

19. A crystal according to claim 16 wherein said ligand binding domain comprises amino acid residues selected from one or more of the following: I255, P253, A252, Y250, P234, P129, G127, L126, Q125, L47, V45, H44, W227 and W364.

20. A crystal according to claim 13, which comprises one or more of the following interactions between human PCNA and residues 3 to 15 of said peptide of formula I: V3(N)-I255(O), L4(N)-I255(O), L4(O)-I255(N), Q5(OE1)-W227(O), Q5(NE2)-A252(O), Q5(NE2)-P253(O), K6(N)-P253(O), K6(CG)-I255(CG1), I8(N)-H44(O), I8(CD1)-P234(CB), I8(CD1)-Y250(CB), I8(CD1)-Y250(C), I8(CG2)-L47(CD1), I8(CG1)-V45(C), Y11(CG)-P234(CD), Y11(CD1)-P234(CD), Y11(CZ)-P234(CD), Y11(CZ)-P234(CG), P12(CD2)-P129(CD), P12(CE2)-P129(CD), P12(CZ)-P234(CG), P12(CZ)-Y250(CD2), P12(CE1)-Y250(CD2), P12(CE1)-W364(O), H13(O)-G127(N), P14(CA)-L126(CD2) and K15(N)-Q125(O).

21. A crystal according to claim 13 wherein said peptide of formula I comprises one or more of the following intramolecular H-bonds: Q5(NE2)-K6(O), K7(O)-D10(N), I8(O)-Y11(N), I8(O)-F2(N) and D10(N)-D10(OD1).

22. A method of screening for a ligand capable of binding to a ligand binding domain, wherein said method comprises the use of a crystal according to claim 1 or the structure co-ordinates of Table 3, Table 4 and/or Table 5.

23. A method of screening for a ligand capable of binding to a ligand binding domain, wherein the ligand binding domain is that defined in claim 19, the method comprising contacting the ligand binding domain with a test compound and determining if said test compound binds to said ligand binding domain.

24. A human PCNA ligand binding domain agonist, wherein said ligand binding domain is that defined in claim 19.

25. A human PCNA binding domain antagonist, wherein said ligand binding domain is that defined in claim 19.

26. A method of screening for a modulator of PCNA, wherein the method comprises using a crystal according to of claim 1, or the structure co-ordinates of Table 3, Table 4 and/or Table 5.

27. A method according to claim 26 comprising the steps of:
(a) providing at least a portion of the structure co-ordinates of Table 3, Table 4 and/or Table 5;
(b) employing at least a portion of the structure co-ordinates of Table 3, Table 4 and/or Table 5 to design or select or synthesise a putative modulator of PCNA;
(c) contacting the putative modulator of PCNA with PCNA or a mutant, variant, homologue, derivative or fragment thereof in the presence of a substrate; and
(d) determining whether said putative modulator of PCNA modulates PCNA.

28. A method according to claim 27 wherein at least a portion of the structure co-ordinates of Table 3, Table 4 and/or Table 5 and/or the putative modulator of PCNA and/or the substrate are provided on a machine-readable data storage medium comprising a data storage material encoded with machine readable data.

29. A method according to claim 27 wherein the putative PCNA modulator is from a library of compounds.

30. A method according claim 27 wherein the putative PCNA modulator is selected from a database.

31. A method according to claim 27 wherein the putative PCNA modulator is designed de novo.

32. A method according to claim 27 wherein the putative PCNA modulator is designed from a known PCNA modulator.

33. A method according to claim 27 wherein the design or selection of the putative PCNA modulator is performed in conjunction with computer modelling.

34. A method according to claim 26 wherein the modulator of PCNA inhibits PCNA activity.

35. A method according to claim 26 wherein the PCNA modulator is useful in the prevention and/or treatment of a PCNA related disorder.

36. An assay for a candidate compound capable of modulating PCNA, said assay comprising the steps of:
(a) contacting said candidate compound with PCNA;
(b) detecting whether said candidate compound forms associations with one or more amino acid residues corresponding to PCNA amino acid residues I255, P253, A252, Y250, P234, P129, G127, L126, Q125, L47, V45, H44, W227 and W364.

37. An assay according to claim 36 wherein said candidate compound is selected by performing rational drug design with a 3-dimensional model of PCNA in conjunction with computer modelling.

38. An assay according to claim 36 which is a competitive binding assay using a known modulator of PCNA.

39. A modulator of PCNA, ligand or candidate compound identified by the method of claim 22.

40. A modulator of PCNA, ligand or candidate compound according to claim 39 which inhibits PCNA activity.

41. A modulator of PCNA, ligand or candidate compound according to claim 39 which selectively modulates PCNA activity.

42. A modulator of PCNA, ligand or candidate compound according to claim 39 which is capable of forming associations with one or more amino acid residues corresponding to I255, P253, A252, Y250, P234, P129, G127, L126, Q125, L47, V45, H44, W227 and W364.

43. A pharmaceutical composition comprising a modulator of PCNA, ligand or compound according to claim 39 and a pharmaceutically acceptable carrier, diluent, excipient or adjuvant or any combination thereof.

44. A method of preventing and/or treating an PCNA related disorder comprising administering a modulator of PCNA, ligand or candidate compound according to claim 39 wherein said modulator of PCNA, ligand or candidate compound, or said pharmaceutical is capable of causing a beneficial preventative and/or therapeutic effect.

45. (canceled)

46. A method according to claim 44 wherein the PCNA dependent disorder is a disorder associated with increased PCNA activity.

47. A method according to claim 44 wherein the PCNA related disorder is a proliferative disorder.

48. A method according to claim 47 wherein the proliferative disorder is selected from cancer, leukemia, glomerulonephritis, rheumatoid arthritis, psoriasis and chronic obstructive pulmonary disorder.

49. A process comprising the steps of:
(a) performing the method of claim 22;
(b) identifying one or more modulators of PCNA; and
(c) preparing a quantity of said one or more PCNA modulators.

50. A process comprising the steps of:
(a) performing the method of claim 22;
(b) identifying one or more PCNA modulators; and
(c) preparing a pharmaceutical composition comprising said one or more identified PCNA modulators.

51. A process comprising the steps of:
(c) performing the method of claim 22;
(b) identifying one or more PCNA modulators;
(c) modifying said one or more PCNA modulators; and
(d) optionally preparing a pharmaceutical composition comprising said one or more PCNA modulators.

52. A computer for producing a three-dimensional representation of PCNA wherein said computer comprises:
(a) a computer-readable data storage medium comprising a data storage material encoded with computer-readable data, wherein said data comprises the structure co-ordinates of Table 3, Table 4 and/or Table 5;
(b) a working memory for storing instructions for processing said computer-readable data;
(c) a central-processing unit coupled to said working memory and to said computer-readable data storage medium for processing said computer-machine readable data into said three-dimensional representation; and
(d) a display coupled to said central-processing unit for displaying said three-dimensional representation.

53. A machine-readable data storage medium comprising a data storage material encoded with machine readable data, wherein the data is defined by at least a portion of the structure co-ordinates of Table 3, Table 4 and/or Table 5.

54. A method of predicting the structure and/or function of potential modulators of PCNA, comprising using the computer of claim 52, such that the structure and/or function of modulators of PCNA are predicted.

55. A method for screening for modulators of PCNA, comprising using at least a portion of the structure co-ordinates of Table 3, Table 4 and/or Table 5, such modulators of PCNA are screened.

56 A method for designing, selecting and synthesizing modulators of PCNA, comprising using at least a portion of the structure co-ordinates of Table 3, Table 4 and/or Table 5 in molecular design techniques, such that modulators of PCNA are designed, selected and synthesized.

57. A method of identifying chemical entities or compounds that modulate PCNA, comprising using at least a portion of the structure co-ordinates of Table 3, Table 4 and/or Table 5 to screen small molecule databases for chemical entities or compounds that modulate PCNA, such that chemical entities or compounds that modulate PCNA are identified.

58. A method according to of claim 54 wherein the modulator of PCNA, chemical entity or compound selectively inhibits the activity of PCNA.

59. A method of solving the structure of a crystalline form of a protein, comprising using at least a portion of the structure co-ordinates of Table 3, Table 4 and/or Table 5 to solve the structure of the crystalline form of a protein, wherein said protein has significant amino acid sequence homology to any functional domain of PCNA.

60. The method according to claim 59 wherein the structure of the crystalline form of a protein with significant amino acid sequence homology to any functional domain of PCNA is solved using molecular replacement.

61. A method of modulating PCNA activity in a cell, said method comprising contacting the cell with a modulator of PCNA according to claim 40.

62. A method according to claim 61 wherein the cell is a cancer cell.

63. A fragment of PCNA, or a homologue, mutant, or derivative thereof, comprising a ligand binding domain, said ligand binding domain being defined by the amino acid residue structural coordinates selected from one or more of the following: I255, P253, A252, Y250, P234, P129, G127, L126, Q125, L47, V45, H44, W227 and W364.

64. A fragment of PCNA, or a homologue, mutant or derivative thereof, according to claim 63 which corresponds to a portion of the structure co-ordinates of Table 3, Table 4 and/or Table 5.

65. Use of a fragment of PCNA, or a homologue, mutant, or derivative thereof, according to claim 63 in an assay for identifying candidate compounds capable of modulating PCNA.

66-69. (canceled)


SEQUENCE LISTING (12 sequences)

SEQ ID NO: 1 - protein, 20 aa, Artificial Sequence
  Description of Artificial Sequence Synthetic peptide
KLFNLHINSAVLQKKITDYF

SEQ ID NO: 2 - protein, 16 aa, Artificial Sequence
  Description of Artificial Sequence Synthetic peptide
MQRSIMSFFHPKKEGK

SEQ ID NO: 3 - protein, 20 aa, Artificial Sequence
  Description of Artificial Sequence Synthetic peptide
KRRQTSMTDFYHSKRRLIFS

SEQ ID NO: 4 - protein, 16 aa, Artificial Sequence
  Description of Artificial Sequence Synthetic peptide
SAVLQKKITDYFHPKK

SEQ ID NO: 5 - protein, 8 aa, Artificial Sequence
  Description of Artificial Sequence Synthetic peptide
QTSMTDFY

SEQ ID NO: 6 - protein, 5 aa, Artificial Sequence
  Description of Artificial Sequence Synthetic peptide
RRLIF

SEQ ID NO: 7 - protein, 9 aa, Artificial Sequence
  Description of Artificial Sequence Synthetic peptide
HSKRRLIFS

SEQ ID NO: 8 - protein, 8 aa, Artificial Sequence
  Description of Artificial Sequence Synthetic peptide
QKKITDYF

SEQ ID NO: 9 - protein, 6 aa, Artificial Sequence
  Description of Artificial Sequence Synthetic 6xHis tag
HHHHHH

SEQ ID NO: 10 - protein, 22 aa, Artificial Sequence
  Description of Artificial Sequence Synthetic peptide
GRKRRQTSMTDFYHSKRRLIFS

SEQ ID NO: 11 - protein, 4 aa, Artificial Sequence
  Description of Artificial Sequence Synthetic peptide
RKRR

SEQ ID NO: 12 - protein, 6 aa, Artificial Sequence
  Description of Artificial Sequence Synthetic peptide
RRLIFS